(19) United States
(12) Patent Application Publication
(10) Pub. No.: US 2025/0064941 A1
(43) Pub. Date: Feb. 27, 2025

(54) SIGLEC LIGANDS, CONJUGATES, AND METHODS OF USE THEREOF

(71) Applicant: Osprey Biopharmaceuticals, Inc., San Diego, CA (US)

(72) Inventors: Mehdi Numa, San Diego, CA (US); Francois Vermersch, San Diego, CA (US); Garrick Packard, San Diego, CA (US); Grant Gabrelow, San Diego, CA (US)

(21) Appl. No.: 18/741,512

(22) Filed: Jun. 12, 2024

Related U.S. Application Data
(60) Provisional application No. 63/521,037, filed on Jun. 14, 2023.

(51) Int. Cl. A61K 47/54; A61K 38/16; A61K 47/68; A61P 37/06; C07K 16/42; G01N 33/577

(52) U.S. Cl. CPC A61K 47/545; A61K 38/164; A61K 47/6873; A61P 37/06; C07K 16/4283; G01N 33/577

(57) ABSTRACT

The present disclosure provides Siglec ligands that have particular linkers. Also provided are conjugates that include a biologically active substance that is covalently bonded to the connecting group of the Siglec ligands. For instance, the biologically active substance can be a biotherapeutic or an autoantigen. The linkers can have advantageous properties that improve the technical qualities of the corresponding conjugates.

### CROSS-REFERENCE TO RELATED APPLICATIONS

This application claims the benefit of U.S. Provisional Application No. 63/521,037, filed Jun. 14, 2023, the disclosure of which is incorporated by reference herein in its entirety.

### INTRODUCTION

Autoimmune disorders are caused when the immune system destroys native tissues, cells, or biomolecules by, e.g., mounting an immune response to self-antigens. Immune disorders are often treated with systemic immune suppression, e.g., by administering antibodies, steroids, gene therapy, etc. Such treatments are not always target specific and/or effective, resulting in compromised immune responses and side effects. Such treatments, when effective, may only be effective transiently.

Therapeutic proteins and gene therapies are novel and successful drug modalities for the treatment of disease. However, patient immune responses to such therapeutics often result in inhibition of drug activity, accelerated drug clearance, compromised drug safety, and loss of drug efficacy. Prevention of the formation of neutralizing and non-neutralizing drug-specific antibodies (“anti-drug antibodies” or “ADA”) is a key unsolved problem in the field of biotherapeutics. Blocking ADA responses to biotherapeutics would improve drug exposure, improve durability of efficacy, reduce ADA-related toxicities, and enable favorable pharmacology for otherwise undruggable modalities (e.g., de novo designed drugs, drugs based on endogenous proteins).

### SUMMARY

The present disclosure provides Siglec ligands that have particular linkers. Also provided are conjugates that include a biologically active substance that is covalently bonded to the linker of the Siglec ligands. For instance, the biologically active substance can be a biotherapeutic or an autoantigen. The linkers can have advantageous properties that improve the technical qualities of the corresponding conjugates.

### BRIEF DESCRIPTION OF THE DRAWINGS

FIG. 1 describes measurement of the Siglec receptor binding specificity of Siglec ligands. FIG. 1A describes the method for the flow cytometry-based competitive Siglec selectivity assay where biotinylated CD22 ligand is immobilized on a streptavidin bead. A fixed concentration of Human Siglec-2-Fc-AF647 is then queried for competitive binding to CD22 ligand-bead. FIG. 1B shows the results of the competitive binding experiment at high concentration of Siglec protein competitor.

FIGS. 2A and 2B depict an in vitro primary mouse B cell activation assay testing for the importance of CD22 engagement for Siglec Ligand-conjugate-mediated suppression of B cell receptor activation, according to embodiments of the present disclosure.

FIG. 3A demonstrates the suppressive effects on human B cell activation with anti-human-IgM CD22 ligand conjugates on primary human PBMC-derived B cells. FIG. 3B shows the competitive cell binding of anti-IgM-CD22 ligand conjugates in comparison to unmodified anti-IgM antibody.

FIG. 4 shows the ADA titers of mice dosed at 10, 3, and 0.3 mg/kg at day 20, post 3rd dose (FIG. 4A) and day 27, post 4th dose (FIG. 4B) with adalimumab control versus CD22 ligand conjugated adalimumab.

FIG. 5A depicts the clearance rates of adalimumab control versus CD22 ligand conjugated adalimumab. FIG. 5B shows the exposure, as measured by pharmacokinetic area under the curve values for the PK of each adalimumab-Siglec Ligand conjugate, showing the AUC value for each mouse in the study.

### DETAILED DESCRIPTION

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. It is further noted that the claims may be drafted to exclude any element, e.g., any optional element. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Further, the dates of publication provided may be different from the actual publication dates which may need to be independently confirmed. To the extent the definition or usage of any term herein conflicts with a definition or usage of a term in an application or reference incorporated by reference herein, the instant application shall control.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Definitions

### Chemical Groups

“Alkyl” refers to a monoradical, branched or linear, non-cyclic, saturated hydrocarbon group. Exemplary alkyl groups include methyl, ethyl, n-propyl, isopropyl, n-butyl, isobutyl, t-butyl, octyl, decyl, cyclopentyl, and cyclohexyl. In some cases, the alkyl group has 1 to 24 carbon atoms, e.g. 1 to 12, 1 to 6, or 1 to 3.

“Alkenyl” refers to a monoradical, branched or linear, non-cyclic hydrocarbonyl group that comprises a carbon-carbon double bond. Exemplary alkenyl groups include ethenyl, n-propenyl, isopropenyl, n-butenyl, isobutenyl, octenyl, decenyl, tetradecenyl, hexadecenyl, eicosenyl, and tetracosenyl.

“Alkynyl” refers to a monoradical, branched or linear, non-cyclic hydrocarbonyl group that comprises a carbon-carbon triple bond. Exemplary alkynyl groups include ethynyl and n-propynyl.

“Cycloalkyl” refers to a monoradical, cyclic, saturated hydrocarbon group. Similarly, “cycloalkenyl” refers to a monoradical and cyclic group having carbon-carbon double bond whereas “cycloalkynyl” refers to a monoradical and cyclic group having carbon-carbon triple bond.

“Heterocyclyl” refers to a monoradical, cyclic group that contains a heteroatom (e.g. O, S, N) as a ring atom and that is not aromatic (i.e. distinguishing heterocyclyl groups from heteroaryl groups). Exemplary heterocyclyl groups include piperidinyl, tetrahydrofuranyl, dihydrofuranyl, and thiocanyl.

“Aryl” refers to an aromatic group containing at least one aromatic ring, wherein each of the atoms in the ring are carbon atoms, i.e. none of the ring atoms are heteroatoms (e.g. O, S, N). In some cases the aryl group has a second aromatic ring, e.g. that is fused to the first aromatic ring. Exemplary aryl groups are phenyl, naphthyl, biphenyl, diphenylether, diphenylamine, and benzophenone.

“Heteroaryl” refers to an aromatic group containing at least one aromatic ring, wherein at least one of the atoms in the aromatic ring is a heteroatom (e.g. O, S, N). Exemplary heteroaryl groups include those obtained from removing a hydrogen atom from pyridine, pyrimidine, furan, thiophene, or benzothiophene.

The term “substituted” refers the removal of one or more hydrogens from an atom (e.g. from a C or N atom) and their replacement with a different group. For instance, a hydrogen atom on a phenyl (—C₆H₅) group can be replaced with a methyl group to form a —C₆H₄CH₃ group. Thus, the —C₆H₄CH₃ group can be considered a substituted aryl group. As another example, two hydrogen atoms from the second carbon of a propyl (—CH₂CH₂CH₃) group can be replaced with an oxygen atom to form a —CH₂C(O)CH₃ group, which can be considered a substituted alkyl group. However, replacement of a hydrogen atom on a propyl (—CH₂CH₂CH₃) group with a methyl group (e.g. giving —CH₂CH(CH₃)CH₃) is not considered a “substitution” as used herein since the starting group and the ending group are both alkyl groups. However, if the propyl group was substituted with a methoxy group, thereby giving a —CH₂CH(OCH₃)CH₃ group, the overall group can no long be considered “alkyl”, and thus is “substituted alkyl”. Thus, in order to be considered a substituent, the replacement group is a different type than the original group. In addition, groups are presumed to be unsubstituted unless described as substituted. For instance, the term “alkyl” and “unsubstituted alkyl” are used interchangeably herein.

Exemplary substituents include alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof.

In some cases, the substitutions can themselves be further substituted with one or more groups. For example, the group —C₆H₄CH₂CH₃ can be considered as substituted aryl, i.e. an aryl group substituted with the ethyl, which is an alkyl group. Furthermore, the ethyl group can itself be substituted with a pyridyl group to form —C₆H₄CH₂CH₂C₅H₅N, wherein —C₆H₄CH₂CH₂C₅H₅N can also be considered as a substituted aryl group as the term is used herein. In some cases, the substituents are not substituted with any other groups.

Monoradical and multiradical groups are described herein. For example, the methyl group (—CH₃) and the ethyl group (—CH₂CH₃) are monoradical groups. In contrast, exemplary diradical groups include diylmethane (—CH₂—, which is also known as a methylene group) and 1,2-diylethane (—CH₂CH₂—). The term “arylene” refers to the diradical version of an aryl group, e.g. 1,4-diylbenzene refers to a C₆H₄ fragment wherein two hydrogens that are located para to one another are removed and replaced with single bonds to other groups. The terms “alkenylene”, “alkynylene”, “heteroarylene”, and “heterocyclene” are also used herein. The term “diradical connector” is used interchangeably with the term “diradical group”. Exemplary diradical groups include alkyl groups, substituted alkyl groups, polyethylene glycol groups, alkoxy groups, substituted alkoxy groups, arylalkyl groups, and substituted arylalkyl groups.

“Acyl” refers to a group of formula —C(O)R wherein R is alkyl, alkenyl, alkynyl, or substituted versions thereof. For example, the acetyl group has formula —C(O)CH₃. “Carbonyl” refers to a diradical group of formula —C(O)—.

“Alkoxy” refers to a group of formula —O(alkyl). Similar groups can be derived from alkenyl, alkynyl, aryl, heteroaryl, and other groups.

“Amino” refers to the group —NR^{X}R^{Y} wherein R^{X} and R^{Y} are each independently H or a non-hydrogen substituent. Exemplary non-hydrogen substituents include alkyl groups (e.g. methyl, ethyl, and isopropyl) and the carbonyl group.

“Amide” refers to the group —C(O)NR^{Q}R^{R} wherein R^{Q} and R^{R} are each independently H or a non-hydrogen substituent.

“Carbonyl” refers to a diradical group of formula —C(O)—.

“Carboxy” is used interchangeably with carboxyl and carboxylate to refer to the —CO₂H group and salts thereof.

“Ether” refers to a diradical group of formula —O—. For instance, if the ether group is connected to an alkyl group, then the overall group is an alkoxy group (e.g. —OCH₃ or methoxy). If the ether is connected to a carbonyl group, then the overall group is an ester group of formula —OC(O)—.

“Halo” and “halogen” refer to the chloro, bromo, fluoro, and iodo groups.

“Nitro” refers to the group of formula —NO₂.

“Oxamide” refers to the group —R^{P}N—C(O)C(O)—NR^{P}R^{Q} wherein R^{P} and R^{Q} are each independently H or a non-hydrogen substituent. “Oxamide” can also be referred to as ethanediamide or oxalamide.

“Phosphoramide” refers to the group —HNP(O)(NH₂)₂. “Phosphoramide” can also be referred to as phosphoric triamide.

“Sulfonamide” refers to the group —R^{P}N—S(O)₂—NR^{P}R^{Q} wherein R^{P} and R^{Q} are each independently H or a non-hydrogen substituent. “Sulfonamide” can also be referred to as Sulfuric diamide.

“Thioamide” refers to the group —RPS(O)NR^{P}R^{Q} wherein R^{P}, R^{Q} and R^{R} are each independently H or a non-hydrogen substituent. “Thioamide” can also be referred to as alkylsulfnamide.

“Ureido” refers to univalent radical of urea. In other words, “ureido” refers to the group —HNC(O)NHR^{R}, wherein R^{R} is H or a non-hydrogen substituent. “Ureido” can also be referred to as urea.

Unless otherwise specified, reference to an atom is meant to include all isotopes of that atom. For example, reference to H includes ¹H, ²H (i.e. D or deuterium) and ³H (i.e. tritium), and reference to C is includes both ¹²C and all other isotopes of carbon (e.g. ¹³C). Unless specified otherwise, groups include all possible stereoisomers.

“Chemoselective functional group” refers to a functional group that can selectively react with another compatible functional group to form a covalent bond, in some cases, after optional activation of one of the functional groups. Chemoselective functional groups of interest include, but are not limited to, thiols and maleimide or iodoacetamide, amines and carboxylic acids or active esters thereof, as well as groups that can react with one another via Click chemistry, e.g., azide and alkyne groups (e.g., cyclooctyne groups), tetrazine, transcyclooctene, dienes and dienophiles, and azide, sulfur(VI) fluoride exchange chemistry (SuFEX), sulfonyl fluoride, as well as hydroxyl, hydrazido, hydrazino, aldehyde, ketone, azido, alkyne, phosphine, epoxide, succinimide, pentafluorophenyl (PFP) ester, and carboxylic acid (e.g. on a lysine residue).

### Treatment

The terms active agent, active pharmaceutical ingredient, pharmacologically active agent, and drug are used interchangeably herein to refer to a chemical material or compound which, when administered to an organism (human or animal) induces a desired pharmacologic and/or physiologic effect by local and/or systemic action.

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; and the like. “Mammal” means a member or members of any mammalian species, and includes, by way of example, canines; felines; equines; bovines; ovines; rodentia, etc. and primates, e.g., non-human primates, and humans. Non-human animal models, e.g., mammals, e.g. non-human primates, murines, lagomorpha, etc. may be used for experimental investigations.

As used herein, the terms “treatment,” “treating,” and the like, refer to obtaining a desired pharmacologic and/or physiologic effect, such as reduction of viral titer. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease (as in liver fibrosis that can result in the context of chronic HCV infection); (b) inhibiting the disease, i.e., arresting its development; and (c) relieving the disease, i.e., causing regression of the disease (e.g., reduction in viral titers)

A “therapeutically effective amount”, a “therapeutically effective dose” or “therapeutic dose” is an amount sufficient to effect desired clinical results (i.e., achieve therapeutic efficacy, achieve a desired therapeutic response, etc.). A therapeutically effective dose can be administered in one or more administrations. For purposes of this disclosure, a therapeutically effective dose of a compositions is an amount that is sufficient, when administered to the individual, to palliate, ameliorate, stabilize, reverse, prevent, slow or delay the progression of a disease state (e.g., cancer, etc.) present in the subject.

As used herein, the terms “determining,” “measuring,” “assessing,” and “assaying” are used interchangeably and include both quantitative and qualitative determinations.

The term “unit dosage form,” as used herein, refers to physically discrete units suitable as unitary dosages for human and animal subjects, each unit containing a predetermined quantity of a compound (e.g., an aminopyrimidine compound, as described herein) calculated in an amount sufficient to produce the desired effect in association with a pharmaceutically acceptable diluent, carrier or vehicle. The specifications for unit dosage forms depend on the particular compound employed and the effect to be achieved, and the pharmacodynamics associated with each compound in the host.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

### Methods of Treatment

Provided are methods of treating a subject for a condition by administering a conjugate as described herein. In some cases, the subject has been diagnosed with a condition.

The subject compounds or prodrugs find use for treating a disease or disorder in a subject. The route of administration may be selected according to a variety of factors including, but not limited to, the condition to be treated, the formulation and/or device used, the subject to be treated, and the like. Routes of administration useful in the disclosed methods include, but are not limited to, oral and parenteral routes, such as intravenous (iv), intraperitoneal (ip), rectal, topical, ophthalmic, nasal, otic, intrathecal, and transdermal. Formulations for these dosage forms are described herein.

An effective amount of a subject compound or prodrug may depend, at least, on the particular method of use, the subject being treated, the severity of the affliction, and the manner of administration of the therapeutic composition. A “therapeutically effective amount” of a composition is a quantity of a specified compound or prodrug sufficient to achieve a desired effect in a subject (e.g., patient) being treated. For example, this may be the amount of a subject compound necessary to prevent, inhibit, reduce or relieve a disease or disorder in a subject. Ideally, a therapeutically effective amount of a compound or prodrug is an amount sufficient to prevent, inhibit, reduce or relieve a disease or disorder in a subject without causing a substantial cytotoxic effect on host cells in the subject.

Therapeutically effective doses of a subject compound or prodrug or pharmaceutical composition can be determined by one of skill in the art. For example, in some instances, a therapeutically effective dose of a compound or prodrug or pharmaceutical composition is administered with a goal of achieving local (e.g., tissue) concentrations that are at least as high as the IC₅₀ of an applicable compound disclosed herein.

The specific dose level and frequency of dosage for any particular subject may be varied and may depend upon a variety of factors, including the activity of the subject compound or prodrug, the metabolic stability and length of action of that compound or prodrug, the age, body weight, general health, sex and diet of the subject, mode and time of administration, rate of excretion, drug combination, and severity of the condition of the host undergoing therapy.

In some embodiments, multiple doses of a compound or prodrug are administered. The frequency of administration of a compound can vary depending on any of a variety of factors, e.g., severity of the symptoms, condition of the subject, etc. For example, in some embodiments, a compound is administered once per month, twice per month, three times per month, every other week, once per week (qwk), twice per week, three times per week, four times per week, five times per week, six times per week, every other day, daily (qd/od), twice a day (bds/bid), or three times a day (tds/tid), etc.

### Compositions and Formulations

Also provided are compositions comprising a pharmaceutically active compound. In some cases, the composition includes a racemic mixture of stereoisomers. In some embodiments, the composition is enriched in a particular stereoisomer, e.g. the composition is enriched in a first enantiomer relative to a second enantiomer. The term “enantiomeric excess” is used herein to quantify the relative amount of the first enantiomer compared to the second enantiomer, wherein enantiomeric excess is the absolute difference between the mole fraction of each enantiomer. For instance, if 70% of a compound is a first enantiomer and 30% of the compound is the second enantiomer, then the enantiomeric excess is 40% (i.e. 70% minus 40%). In some cases, the composition has an enantiomeric excess of the first enantiomer of 1% or more, such as 10% or more, 20% or more, 30% or more, or 40% or more. In some embodiments, the composition is an aqueous solution of the compound.

In certain embodiments, the disclosed compounds and prodrugs thereof are useful for the treatment of a disease or disorder. Accordingly, pharmaceutical compositions comprising at least one disclosed compound or prodrug are also described herein. For example, the present disclosure provides pharmaceutical compositions that include a therapeutically effective amount of a compound or prodrug of the present disclosure (or a pharmaceutically acceptable salt or solvate or hydrate or stereoisomer thereof) and a pharmaceutically acceptable excipient.

A pharmaceutical composition that includes a subject compound (or prodrug) may be administered to a patient alone, or in combination with other supplementary active agents. For example, one or more compounds or prodrugs according to the present disclosure can be administered to a patient with or without supplementary active agents. The pharmaceutical compositions may be manufactured using any of a variety of processes, including, but not limited to, conventional mixing, dissolving, granulating, dragee-making, levigating, emulsifying, encapsulating, entrapping, lyophilizing, and the like. The pharmaceutical composition can take any of a variety of forms including, but not limited to, a sterile solution, suspension, emulsion, spray dried dispersion, lyophilisate, tablet, microtablets, pill, pellet, capsule, powder, syrup, elixir or any other dosage form suitable for administration.

A compound or prodrug of the present disclosure may be administered to a subject using any convenient means capable of resulting in the desired reduction in disease condition or symptom. Thus, a compound or prodrug can be incorporated into a variety of formulations for therapeutic administration. More particularly, a compound or prodrug can be formulated into pharmaceutical compositions by combination with appropriate pharmaceutically acceptable excipients, carriers or diluents, and may be formulated into preparations in solid, semi-solid, liquid or gaseous forms, such as tablets, capsules, powders, granules, ointments, creams, gels, foams, solutions, suppositories, injections, inhalants, aerosols, and the like.

Formulations for pharmaceutical compositions are described in, for example, Remington's Pharmaceutical Sciences, by E. W. Martin, Mack Publishing Co., Easton, Pa., 19th Edition, 1995, which describes examples of formulations (and components thereof) suitable for pharmaceutical delivery of the disclosed compounds. Pharmaceutical compositions that include at least one of the compounds or prodrugs can be formulated for use in human or veterinary medicine. Particular formulations of a disclosed pharmaceutical composition may depend, for example, on the mode of administration and/or on the location of the subject to be treated. In some embodiments, formulations include a pharmaceutically acceptable excipient in addition to at least one active ingredient, such as a compound of the present disclosure. In other embodiments, other medicinal or pharmaceutical agents, for example, with similar, related or complementary effects on the disease or condition being treated can also be included as active ingredients in a pharmaceutical composition.

Pharmaceutically acceptable carriers useful for the disclosed methods and compositions may depend on the particular mode of administration being employed. In addition to biologically neutral carriers, pharmaceutical compositions to be administered can optionally contain non-toxic auxiliary substances (e.g., excipients), such as wetting or emulsifying agents, preservatives, and pH buffering agents, and the like. The disclosed pharmaceutical compositions may be formulated as a pharmaceutically acceptable salt of a disclosed compound.

The term “unit dosage form,” as used herein, refers to physically discrete units suitable as unitary dosages for human and animal subjects, each unit containing a predetermined quantity of a compound or prodrug calculated in an amount sufficient to produce the desired effect in association with a pharmaceutically acceptable diluent, excipient, carrier or vehicle. The specifications for a compound or prodrug depend on the particular compound or prodrug employed and the effect to be achieved, and the pharmacodynamics associated with each compound in the subject.

The dosage form of a disclosed pharmaceutical composition may be determined by the mode of administration chosen. For example, in addition to injectable fluids, topical or oral dosage forms may be employed. Topical preparations may include eye drops, ointments, sprays and the like. Oral formulations may be liquid (e.g., syrups, solutions or suspensions), or solid (e.g., powders, pills, tablets, or capsules). Methods of preparing such dosage forms are known, or will be apparent, to those skilled in the art.

Certain embodiments of the pharmaceutical compositions that include a subject compound or prodrug may be formulated in unit dosage form suitable for individual administration of precise dosages. The amount of active ingredient administered may depend on the subject being treated, the severity of the affliction, and the manner of administration, and is known to those skilled in the art. In certain instances, the formulation to be administered contains a quantity of the compound or prodrug disclosed herein in an amount effective to achieve the desired effect in the subject being treated.

Each therapeutic compound can independently be in any dosage form, such as those described herein, and can also be administered in various ways, as described herein. For example, the compounds or prodrugs may be formulated together, in a single dosage unit (that is, combined together in one form such as capsule, tablet, powder, or liquid, etc.) as a combination product. Alternatively, when not formulated together in a single dosage unit, an individual compound or prodrug may be administered at the same time as another therapeutic compound or sequentially, in any order thereof.

A disclosed compound can be administered alone, as the sole active pharmaceutical agent, or in combination with one or more additional compounds or prodrugs of the present disclosure or in conjunction with other agents. When administered as a combination, the therapeutic agents can be formulated as separate compositions that are administered simultaneously or at different times, or the therapeutic agents can be administered together as a single composition combining two or more therapeutic agents. Thus, the pharmaceutical compositions disclosed herein containing a compound of the present disclosure optionally include other therapeutic agents. Accordingly, certain embodiments are directed to such pharmaceutical compositions, where the composition further includes a therapeutically effective amount of an agent selected as is known to those of skill in the art.

### Siglec Ligands and Conjugates

The term “Siglec” as used herein refers to cell surface proteins that binds to a sialic acid group. Siglecs are commonly found on the surface of leukocytes.

There are 14 different mammalian Siglecs, which are expressed on different types of leukocytes and which may exert inhibitory or activating effects on the cells on which they are expressed depending on whether they comprise an inhibitory motif or activating motif. Siglecs show distinct binding preferences for different sialic acids, and the type of linkage and type of underlying sugar also affect recognition of sialic acids. (Varki, A. and Crocker, P. R. (2009) I-type lectins. In Essentials of Glycobiology (2nd edn) (Varki, A. et al., eds), pp. 459-474, Cold Spring Harbor Laboratory Press; Crocker, P. R. et al. (2007) Nat. Rev. Immunol. 7, 255-266). Together, this provides for an array of alternative Siglec ligands that may be deployed to modulate an immune response to a biotherapeutic.

Provided by the present disclosure are Sialic acid-binding immunoglobulin-type lectin ligands, which are also referred to herein as “Siglec ligands”.

### Siglec Ligands of Formula (I) and Conjugates of Formula (II)

In some cases, the Siglec ligand provided by the present disclosure has formula (I):

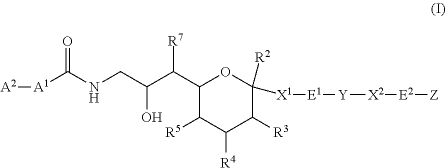

wherein:
A¹ is absent or alkylene;
A² is absent, alkyl, aryl, heteroaryl, cycloalkyl, or a substituted version thereof;
R² is —COOH, —P(O)(OH)₂, —OSO₂OH, —C(O)NHSO₂H, tetrazole, an ester, or a substituted version thereof;
R³ is H, alkyl, alkoxy, —S(alkyl), amino, halo, cyano, ester, amide, or a substituted version thereof;
R⁴ is hydroxy, alkoxy, amino, amide, or a substituted version thereof;
R⁵ is H, amino, ureido, amide, thioamide, oxamide, —NH(cyclobut-3-ene-1,2-dione), sulfonaamide, phosphoramide, alkylamine, triazole, tetrazole, or substituted version thereof;
R⁷ is hydroxy or halo;
X¹ is O, S, triazole, —CH(OH)—, —CH₂—, —CHF—, —NH—, or —CF₂—;
E¹ is absent, —(CH₂CH₂O)_{g}—, —(CH₂)_{g}—, benzyl, cycloalkyl, or a substituted version thereof, wherein g is an integer ranging from 1 to 10;
Y is absent, alkylene, phenylene, substituted phenylene, —O—, —NH—, —S—, —C≡C—, —CH₂C≡C—, —C(O)—, —OP(O)O—, —NH-(cyclobut-3-ene-1,2-dione)-NH—, —NHC(O)—, triazole, —(CH₂CH₂O)ₙ—, —(CH₂)ₙ—, wherein n is an integer ranging from 1 to 10;
X² is absent, O, —NH—, —C(O)NH—, —C(O)—, —C(O)O—, —NHC(O)—, —NHC(O)O—, —OP(O)(OH)O—, —NHC(O)NH—, —(CH₂)ₘ—, —(CH₂CH₂O)ₘ—, —(CH₂CH₂O)ₘNHC(O)—, triazole, or a substituted version thereof, wherein m is an integer ranging from 1 to 10;
E² is absent, —(CH₂)ₕ—, —(CH₂CH₂O)ₕ—, —(CH₂CH₂O)ₕC(O)NH—, —(CH₂CH₂O)ₕNHC(O)—, -(alkylene)C(O)NH(alkylene)-, -(alkylene)C(O)NH(substituted alkylene)-, -(alkylene)C(O)N(substituted alkylene)₂-, —C≡C—, or —CH₂C≡C—, or a substituted version thereof, wherein h is an integer ranging from 1 to 10; and
Z is Z1 or Z1′-T-Z2, wherein Z1 is a chemoselective functional group, wherein Z1′ is a diradical group, T is absent or a diradical group, and Z2 is a chemoselective functional group, a salt thereof, or a stereoisomer thereof.

As discussed above, R² can be —COOH, —P(O)(OH)₂, —OSO₂OH, —C(O)NHSO₂H, tetrazole, an ester or a substituted version thereof. For example, if R² is an ester of —COOH, then R² could be —C(O)O(alkyl) such as C(O)O(ethyl). Similarly, R² could be an ester of —P(O)(OH)₂, such as —P(O)(OH)O(alkyl) such as P(O)(OH)O(propyl)

As discussed above, the Siglec ligand can have formula (I), or it can be salt of formula (I) or a stereoisomer of formula (I). For example, if R² is —COOH then the R² group could be —COO⁻ Na⁺, which would be considered herein as a salt of formula (I). As another example, R⁵ is described as amino group, such as unsubstituted —NH₂. If R⁵ is —NH₃⁺Cl⁻, then the compound would be a salt of formula (I). By “stereoisomer thereof”, it is meant that a composition could include primarily a particular stereoisomer. For example, the carbon atom that is located at the intersection of the C(R⁵) and C(R⁷) and O groups is a chiral center, and a composition could include primarily one of the two possible stereoisomers at that chiral center.

As discussed above, Z1 is a chemoselective functional group and Z1′ is a diradical functional group. For example, Z1′ can be a diradical group that is generated when a chemoselective functional group (Z1) is reacted with a corresponding group to form a covalent bond. For example, if Z1 is an alkyne then the Z1 group can be reacted with an azide group through an azide-alkyne Huisgen cycloaddition to form Z1′ triazole group that connects the E² group to another group. As another example, Z1 can be an alkene group that can reacted with a corresponding tetrazole group through a retro Diels-Alder reaction to form a covalent connection through a pyridazine Z1′ group. T can be absent or a diradical group. For example, T can comprise a polyethylene glycol (PEG) group. As such, in some cases the Siglec ligand terminates with a Z1 chemoselective group that can selectively react and form a bond with another group. For instance, Z1 can react with another molecule of formula Z1*-T-Z2, wherein Z1 and Z1* can chemoselectively react to form Z1′, thereby giving a group of formula —Z1′-T-Z2. As such, Z1* is chemoselective partner group to Z1, e.g. Z1 can be azide and Z1* can be alkyne. This methodology allows for modular attachment and usage of chemoselective groups Z1 and Z2.

Additionally, in some embodiments of formula (I), the Siglec ligand has:
A¹ is absent or alkylene;
A² is absent, aryl, heteroaryl, cycloalkyl, or a substituted version thereof;
R² is —COOH, —P(O)(OH)₂, —OSO₂OH, —C(O)NHSO₂H, tetrazole, an ester or a substituted version thereof;
R³ is H, alkoxy, amino, or a substituted version thereof;
R⁴ is hydroxy, alkoxy, amino, amide, or a substituted version thereof;
R⁵ is amino, ureido, amide, thioamide, oxamide, —NH(cyclobut-3-ene-1,2-dione), sulfonamide or substituted version thereof;
R⁷ is hydroxy or halo;
X¹ is O, S, or triazole;
E¹ is absent or —(CH₂)_{g}— or —(CH₂CH₂O)_{g}—, or a substituted version thereof wherein g is an integer ranging from 1 to 10;
Y is alkylene, phenylene, or substituted phenylene;
X² is absent or O; and
E² is absent, —(CH₂)ₕ—, —(CH₂CH₂O)ₕ—, (alkylene)C(O)NH(alkylene)-, -(alkylene)C(O)N(substituted alkylene)₂-, or (alkylene)C(O)NH(substituted alkylene)-, wherein h is an integer ranging from 1 to 10.

In the terminology of formula (I), the “linker” referred to above is the “X¹-E¹-Y—X²-E²” moiety. As such, the “X¹-E¹-Y—X²-E²” group can have advantageous properties that improve the technical qualities of a conjugate.

In some embodiments of formula (I), the Siglec ligand has:
A¹ is absent;
A² is alkyl;
R² is —COOH;
R³ is H;
R⁴ is hydroxy;
R⁵ is ureido;
R⁷ is hydroxy.

In some embodiments of formula (I), the Siglec ligand has:
A¹ is absent;
A² is substituted alkyl;
R² is tetrazole;
R³ is H;
R⁴ is hydroxy;
R⁵ is amide;
R⁷ is hydroxy.

In some embodiments of formula (I), the Siglec ligand has:
A¹ is absent;
A² is substituted alkyl;
R² is —C(O)NHSO₂H;
R³ is H;
R⁴ is hydroxy;
R⁵ is oxamide;
R⁷ is hydroxy.

In some embodiments, the Siglec ligands of the formula (I) are those of the formulae (Ia)-(Ie), where the symbols have the definitions and preferences indicated in the formula (I).

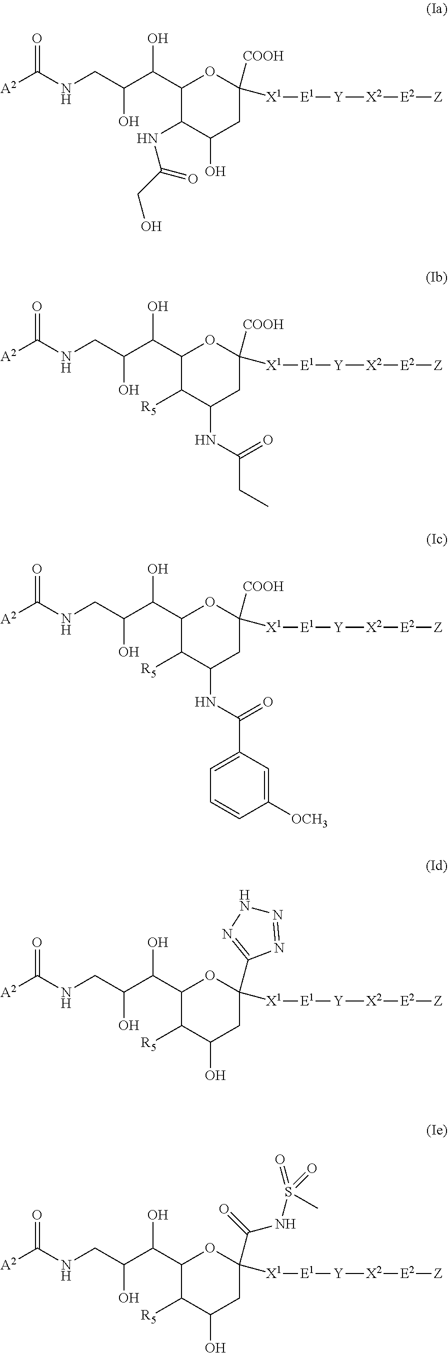

Provided by the present disclosure are conjugates that include a Siglec ligand of formula (I) and a biologically active substance (BAS). Provided by the current disclosure are Siglec conjugates of formula (II):

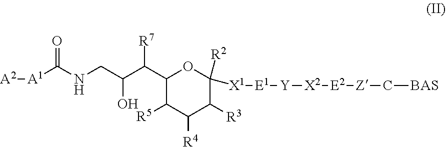

wherein:
A¹ is absent or alkylene;
A² is absent, aryl, heteroaryl, cycloalkyl, or a substituted version thereof;
R² is —COOH, —P(O)(OH)₂, —OSO₂OH, —C(O)NHSO₂H, tetrazole, an ester, or a substituted version thereof;
R³ is H, alkoxy, amino, halo, cyano, ester, amide, or a substituted version thereof;
R⁴ is hydroxy, alkoxy, —S(alkyl), amino, amide, or a substituted version thereof;
R⁵ is H, amino, ureido, amide, thioamide, oxamide, —NH(cyclobut-3-ene-1,2-dione), sulfonaamide, phosphoramide, alkylamine, triazole, tetrazole, or substituted version thereof;
R⁷ is hydroxy or halo;
X¹ is O, S, triazole, —CH(OH)—, —CH₂—, —CHF—, —NH—, or —CF₂—;
E¹ is absent, —(CH₂)_{g}—, —(CH₂CH₂O)_{g}—, benzyl, cycloalkyl, or a substituted version thereof wherein g is an integer ranging from 1 to 10;
Y is absent, alkylene, phenylene, substituted phenylene, —O—, —NH—, —S—, —C≡C—, —CH₂C≡C—, —C(O)—, —OP(O)O—, —NH-(cyclobut-3-ene-1,2-dione)-NH—, —NHC(O)—, triazole, —(CH₂CH₂O)ₙ—, —(CH₂)ₙ—, wherein n is an integer ranging from 1 to 10;
X² is absent, O, —NH—, —C(O)NH—, —C(O)—, —C(O)O—, —NHC(O)—, —NHC(O)O—, —OP(O)(OH)O—, —NHC(O)NH—, —(CH₂)ₘ—, —(CH₂CH₂O)ₘ—, —(CH₂CH₂O)ₘNHC(O)—, triazole, or a substituted version thereof, wherein m is an integer ranging from 1 to 10;
E² is absent, —(CH₂)ₕ—, —(CH₂CH₂O)ₕ—, -(alkylene)C(O)NH(alkylene)-, —(CH₂CH₂O)ₕC(O)NH—, —(CH₂CH₂O)ₕNHC(O)—, (alkylene)C(O)NH(substituted alkylene)-, -(alkylene)C(O)N(substituted alkylene)₂-, —C≡C—, or —CH₂C≡C—, wherein h is an integer ranging from 1 to 10;
Z′ is Z1′ or Z1′-T-Z2′, wherein Z1′ is a diradical group, T is absent or a diradical group, and Z2′ is a diradical group;
C is absent or a diradical group; and
BAS is a biologically active substance,
a salt thereof, or a stereoisomer thereof.

In some cases, C is absent. In such cases, the Siglec ligand terminating with group Z′ is directly covalently bonded to the biologically active substance (BAS). As used herein, both “BAS” and “W” are used interchangeably to refer to the biologically active substance.

In other cases, C is a diradical group. As such, the C group can be referred to as a “connector” that connects the Siglec ligand terminating with Z′ with the biologically active substance. Furthermore, in some cases the C group can be a branched group that connects the biologically active substance to two or more Siglec ligands. For instance, the C group can be a triradical group that connects two Siglec ligands to the biologically active substance. In some cases, the presence of multiple Siglec ligands in the conjugate can increase ability to selectively direct the biologically active substance toward targets that bind to the Siglec ligands.

In some cases, the diradical group Z1′, when starting from the connection to E², can be an alkyl, substituted alkyl, polyethylene glycol, amino, alkoxy, or substituted alkoxy.

In some embodiments of formula (II), the conjugate has:
A¹ is absent or alkylene;
A² is absent, aryl, heteroaryl, cycloalkyl, or a substituted version thereof;
R² is —COOH, —P(O)(OH)₂, —OSO₂OH, —C(O)NHSO₂H, tetrazole, an ester, or a substituted version thereof;
R³ is H, alkoxy, amino, or a substituted version thereof;
R⁴ is hydroxy, alkoxy, amino, amide, or a substituted version thereof;
R⁵ is amino, ureido, amide, thioamide, oxamide, —NH(cyclobut-3-ene-1,2-dione), sulfonamide or substituted version thereof;
R⁷ is hydroxy or halo;
X¹ is O, S, or triazole;
E¹ is absent or —(CH₂)_{g}—, —(CH₂CH₂O)_{g}—, or a substituted version thereof, wherein g is an integer ranging from 1 to 10;
Y is alkylene, phenylene, or substituted phenylene;
X² is absent, or O; and
E² is absent, —(CH₂)ₕ—, —(CH₂CH₂O)ₕ—, -(alkylene)C(O)NH(alkylene)-, -(alkylene)C(O)N(substituted alkylene)₂-, or (alkylene)C(O)NH(substituted alkylene)-, wherein h is an integer ranging from 1 to 10.

In some embodiments of formula (II), the Siglec conjugate has:
A¹ is absent;
A² is alkyl;
R² is —COOH;
R³ is H;
R⁴ is hydroxy;
R⁵ is ureido;
R⁷ is hydroxy.

In some embodiments of formula (II), the Siglec conjugate has:
A¹ is absent;
A² is substituted alkyl;
R² is tetrazole;
R³ is H;
R⁴ is hydroxy;
R⁵ is amide;
R⁷ is hydroxy.

In some embodiments of formula (II), the Siglec conjugate has:
A¹ is absent;
A² is substituted alkyl;
R² is —C(O)NHSO₂H;
R³ is H;
R⁴ is hydroxy;
R⁵ is oxamide;
R⁷ is hydroxy.

In some embodiments, the Siglec conjugates of the formula (II) are those of the formulae (IIa)-(IIe), where the symbols have the definitions and preferences indicated in the formula (II).

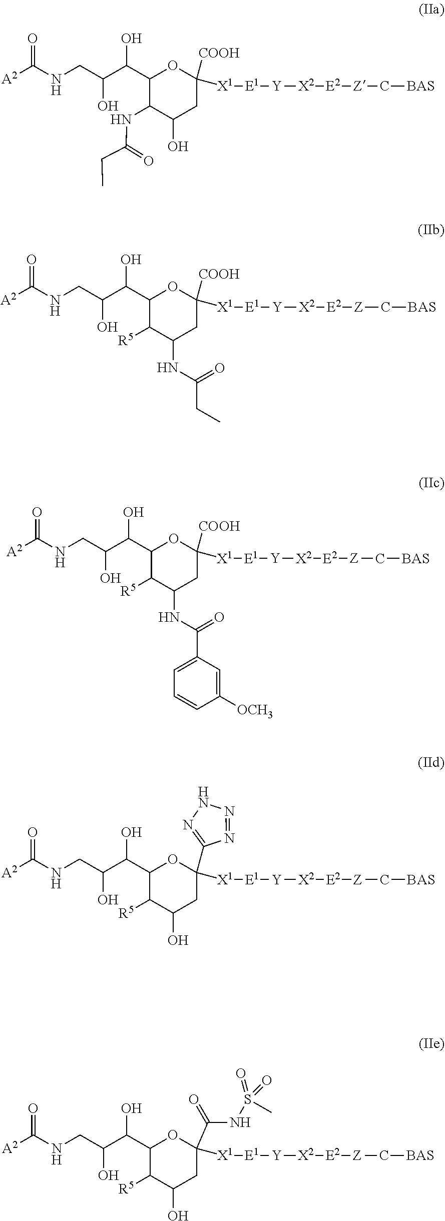

In some embodiments of formula (I) and/or formula (II), A¹ is absent. In some cases, A¹ is alkylene, for example, A¹ is ethylene.

In some embodiments of formula (I) and/or formula (II), A² is alkyl, for example, saturated, straight-chain, or branched C₁-C₁₅ alkyl. In some cases, A² is methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1,1-dimethylpropyl, 1-ethylpropyl, hexyl, 1,1-dimethylbutyl, 2,2-dimethylbutyl, 3,3-dimethylbutyl, 1,2-dimethylbutyl, 1,3-dimethylbutyl, 2,3-dimethylbutyl, 1-methylpentyl, 2-methylpentyl, 3-methylpentyl, 4-methylpentyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl, or octyl.

In some cases, A² is a substituted version of alkyl, for example substituted with C₁-C₈ alkyl, halogen, amino, C₁-C₈ alkyloxy, cyano, nitro, aryl, heteroaryl, or C₃-C₈ cycloalkyl.

In some embodiments of formula (I) and/or formula (II), A² is an aryl, for example, C₆-C₁₄ aryl. In some cases, A² is phenyl, 1-naphthyl, 2-naphthyl, 1-biphenylene, or 2-biphenylene. In some cases, A² is a substituted version of an aryl, for example substituted with C₁-C₈ alkyl, halogen, hydroxy, amino, C₁-C₈ alkyloxy, C₁-C₈ haloalkyl, cyano, nitro, aryl, heteroaryl, or C₃-C₈ cycloalkyl.

In some embodiments of formula (I) and/or formula (II), A² is a heteroaryl for example, 5-10 membered heteroaryl containing one or more heteroatoms selected from S, N, O or SO₂. In some cases, A² is furyl, thienyl, pyrrolyl, pyrazolyl, oxazolyl, pyridinyl, pyrimidinyl, pyrazinyl or 4H-thieno[3,2-c]chromene. In some cases, A² is a substituted version of a heteroaryl, for example substituted with C₁-C₈ alkyl, halogen, amino, C₁-C₈ alkyloxy, cyano, nitro, aryl, heteroaryl, or C₃-C₈ cycloalkyl.

In some embodiments of formula (I) and/or formula (II), A² is a cycloalkyl, for example, C₃-C₈ cycloalkyl. In some cases, A² is cyclopropyl, cyclobutyl, cyclopentyl, cyclohexyl, cycloheptyl or cyclooctyl.

In some cases, A¹ is absent and A² is —CR^{X}R^{Y}R^{Z}.

In some cases, R^{X} is H, F, —CF₃, —OH.

In some cases, R^{Y} is H, F, —OCH₃, —CH₃.

In some cases, R^{Z} is —CH₃. In some cases, R^{Z} is selected from any one of the groups described below:

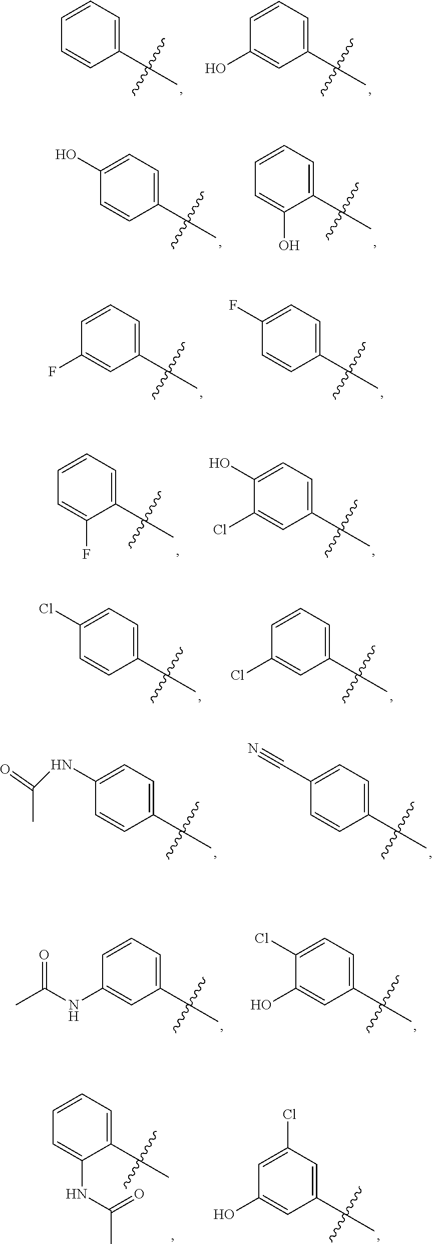

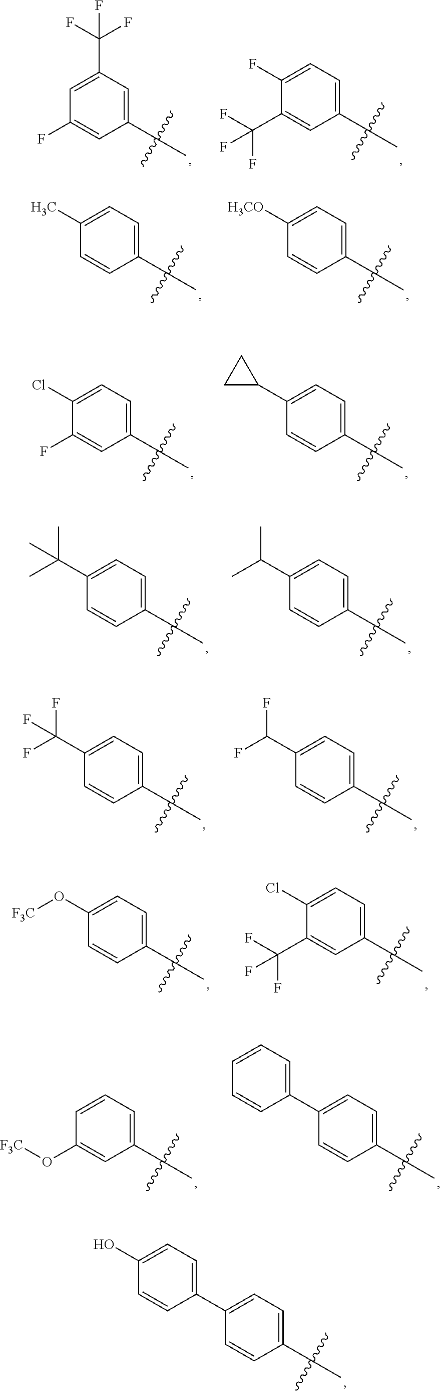

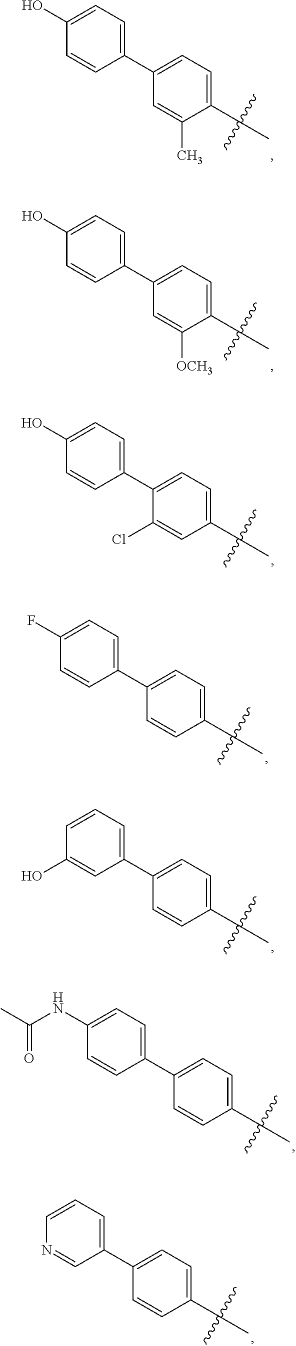

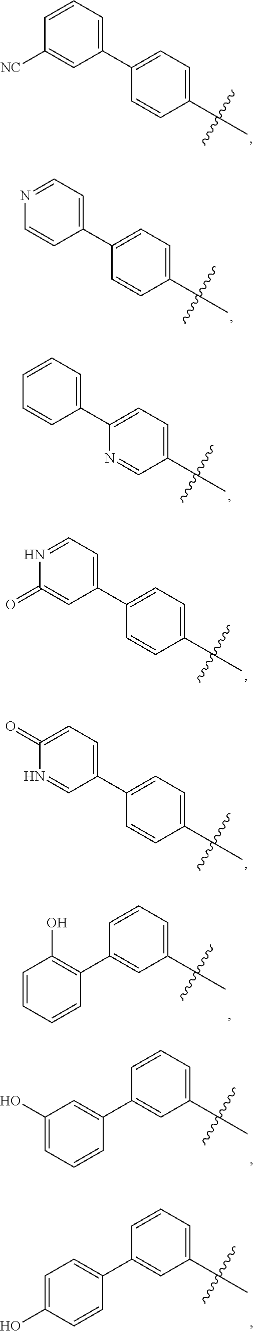

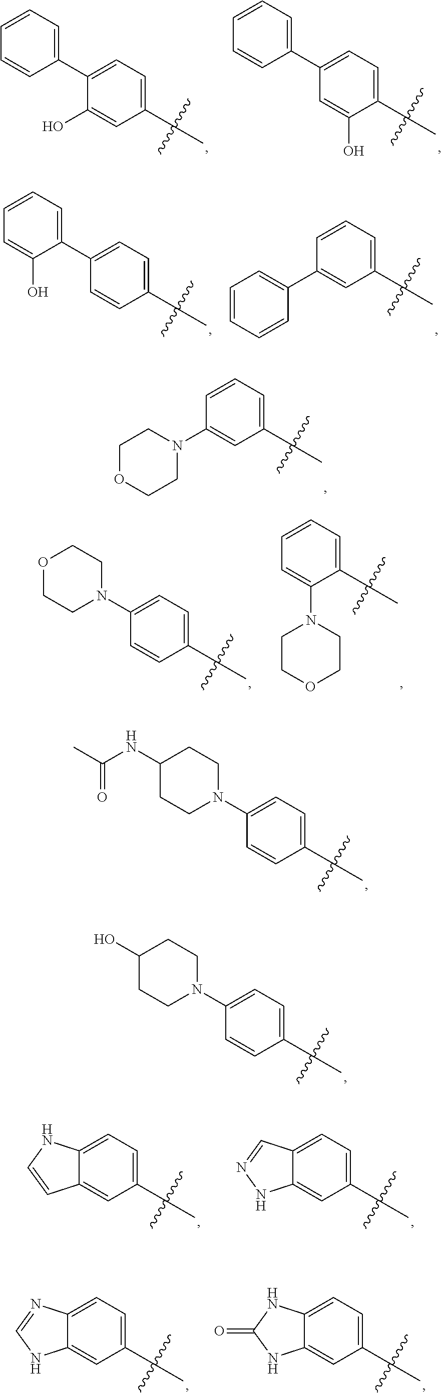

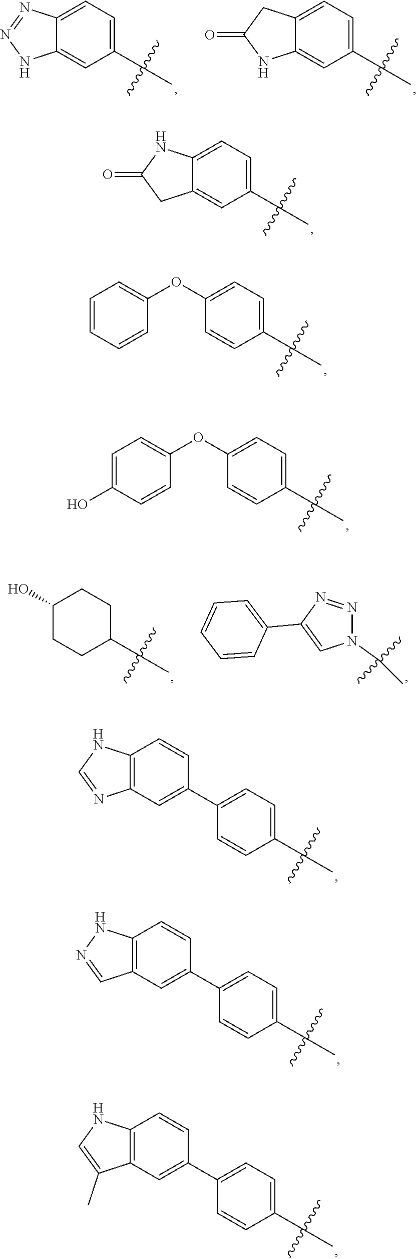

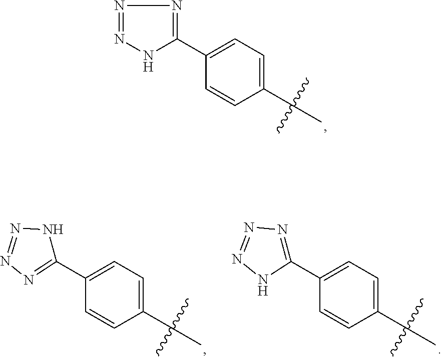 [SMILES: *c1ccc(-c2nnn[nH]2)cc1 ; *c1ccc(-c2nnn[nH]2)cc1 ; *c1ccc(-c2nnn[nH]2)cc1]

In some cases, R^{X} is H, R^{Y} is H and R^{Z} is selected from any of the groups described above.

In some cases, A¹ is absent and A² is cyclopropyl substituted with any one of the groups selected from the following:

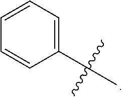 [SMILES: *c1ccccc1]

In some cases, A¹ is absent and A² is tetrahydropyran substituted with any one of the groups selected from the following:

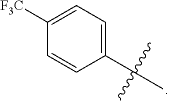 [SMILES: *c1ccc(C(F)(F)F)cc1]

In some embodiments of formula (I) and/or formula (II), R² is —COOH or a substituted version. In some cases, R² is —P(O)(OH)₂ or a substituted version. In some cases, R² is —OSO₂OH or a substituted version. In some cases, R² is C(O)NHSO₂H or a substituted version. In some cases, R² is tetrazole or a substituted version. In some cases, R² is an ester or a substituted version.

In some embodiments of formula (I) and/or formula (II), R³ is H. In some cases, R³ is alkyl, for example, saturated, straight-chain, or branched C₁-C₈ alkyl. In some cases, R³ is alkoxy, for example, saturated, straight-chain, branched or cyclic C₁-C₈ alkoxy. In some cases, R³ is —S(alkyl), for example, —S(C₁-C₈ alkyl). In some cases, R³ is amino or a substituted version. In some cases, R³ is halo, for example fluorine, chlorine, bromine or iodine. In some cases, R³ is cyano. In some cases, R³ is ester or a substituted version. In some cases, R³ is amide or a substituted version.

In some embodiments of formula (I) and/or formula (II), R⁴ is hydroxy. In some cases, R⁴ is alkoxy, for example, saturated, straight-chain, branched or cyclic C₁-C₈ alkoxy. In some cases, R⁴ is amino. In some cases, R⁴ is amide or a substituted version.

In some cases, R⁴ is —NHC(O)CH₂CH₃. In some cases, R⁴ is —NHC(O)C₆H₄OCH₃.

In some embodiments of formula (I) and/or formula (II), R⁵ is amino or a substituted version. In some cases, R⁵ is H. In some cases, R⁵ is ureido or a substituted version. In some cases, R⁵ is amide or a substituted version. In some cases, R⁵ is thioamide or a substituted version. In some cases, R⁵ is oxamide or a substituted version. In some cases, R⁵ is —NH(cyclobut-3-ene-1,2-dione) or a substituted version. In some cases, R⁵ is sulfonamide or a substituted version. In some cases, R⁵ is phosphoramide or a substituted version. In some cases, R⁵ is alkylamine or a substituted version. In some cases, R⁵ is triazole or a substituted version. In some cases, R⁵ is tetrazole or a substituted version.

In some embodiments, R⁵ is —NHC(O)CR^{A}R^{B}R^{C}, —NHC(O)NR^{A}R^{B}, —NHC(O)C(O)NR^{A}R^{B}, —NHC(S)CR^{A}R^{B}R^{C}, —NHC(S)NR^{A}R^{B}, —NR^{A}R^{B}, —NHS(O)₂NR^{A}R^{B}.

In some cases, R^{A} is H, —CH₃, F.

In some cases, R^{B} is H, —CH₃, —C₆H₅, F, —CH₂CF₃, —CH₂CH₂OH, cyclopropyl methyl, —OH, cyclopropyl, —CH₂CH₃, —CH₂C(O)NH₂, —CH₂C(O)NHCH₃, —CH₂C(O)NCH₃CH₃.

In some cases, R^{C} is —OH, F, H, —SH, —OCH₃, —CN, —NHC(O)CH₃, —C₆H₅, —CH₂OH, —NH₂, —NHCH₃, —NCH₃CH₃, —OC₆H₅, —CH₃, tetrazole.

In some embodiments, R⁵ is —NHC(O)C₆H₅.

In some embodiments of formula (I) and/or formula (II), R⁷ is hydroxy. In some cases, R⁷ is halo, for example fluorine, chlorine, bromine or iodine.

In some embodiments of formula (I) and/or formula (II), X¹ is O. In some cases, X¹ is S. In some cases, X¹ is triazole. In some cases, X¹ is —CH(OH)—. In some cases, X¹ is —CH₂—. In some cases, X¹ is —NH—. In some cases, X¹ is —CHF—. In some cases, X¹ is —CF₂—.

In some embodiments of formula (I) and/or formula (II), E¹ is absent. In some cases, E¹ is —(CH₂CH₂O)_{g} or a substituted version. In some cases, E¹ is —(CH₂)_{g}— or a substituted version. In some cases, g is an integer ranging from 1 to 10. In some cases, E¹ is benzyl or a substituted version. In some cases, E¹ is cycloalkyl or a substituted version. For example, E¹ is C₃-C₈ cycloalkyl. In some cases, E¹ is cyclopropyl, cyclobutyl, cyclopentyl, cyclohexyl, cycloheptyl or cyclooctyl.

In some embodiments of formula (I) and/or formula (II), Y is absent. In some cases, Y is alkylene, for example ethylene. In some cases, Y is phenylene. In some cases, Y is a substituted phenylene. In some cases, Y is —C≡C—. In some cases, Y is —CH₂C≡C—. In some cases, Y is —O—. In some cases, Y is —S—. In some cases, Y is —NH—. In some cases, Y is —C(O)—. In some cases, Y is —OP(O)O—. In some cases, Y is —NH-(cyclobut-3-ene-1,2-dione)-NH—. In some cases, Y is —NHC(O)—. In some cases, Y is a triazole. In some cases, Y is —(CH₂CH₂O)ₘ—. In some cases, Y is —(CH₂)ₘ—. In some cases, Y is m is an integer ranging from 1 to 10.

In some embodiments of formula (I) and/or formula (II), X² is absent. In some cases, X² is O. In some cases, X² is —NH—. In some cases, X² is —C(O)NH—. In some cases, X² is —C(O)—. In some cases, X² is —C(O)O—. In some cases, X² is —NHC(O)—. In some cases, X² is —NHC(O)O—. In some cases, X² is —OP(O)(OH)O—. In some cases, X² is —NHC(O)NH—. In some cases, X² is —(CH₂)ₙ—. In some cases, X² is —(CH₂CH₂O)ₙ—. In some cases, X² is —(CH₂CH₂O)ₙNHC(O)—. In some cases, X² is a triazole. In some cases, X² is n is an integer ranging from 1 to 10.

In some embodiments of formula (I) and/or formula (II), E² is absent. In some cases, E² is —(CH₂)ₕ—. In some cases, E² is —(CH₂CH₂O)ₕ—. In some cases, E² is —(CH₂CH₂O)ₕC(O)NH—. In some cases, E² is —(CH₂CH₂O)ₕNHC(O)—. In some cases, E² is -(alkylene)C(O)NH(alkylene)-. In some cases, E² is (alkylene)C(O)NH(substituted alkylene)-. In some cases, E² is -(alkylene)C(O)N(substituted alkylene)₂-. In some cases, E² is —C≡C—. In some cases, E² is —CH₂C≡C—. In some cases, E² is h is an integer ranging from 1 to 10.

In some embodiments of formula (I) and/or formula (II), Z is selected from a group consisting of alkyne, azide, thiol, maleimide, iodoacetamide, amine, carboxylic acid or active ester thereof, tetrazine, trans-cyclooctene, diene, dienophile, hydroxyl, hydrazido, hydrazino, aldehyde, ketone, azido, phosphine, epoxide, succinimide, and phosphate. In some embodiments of formula (I) and/or formula (II), Z is C₂-C₁₀ alkyne, for example, ethyne, propyne, butyne, pentyne, hexyne, heptyne, nonyne or decyne. In some cases, Z is an aryl, for example, C₆-C₁₄ aryl. In some cases, Z is phenyl, 1-naphthyl, 2-naphthyl, 1-biphenylene, or 2-biphenylene. In some cases, Z is a substituted version of an aryl, for example substituted with C₁-C₈ alkyl, halogen, hydroxy, amino, C₁-C₈ alkyloxy, C₁-C₈ haloalkyl, cyano, nitro, aryl, heteroaryl, or C₃-C₈ cycloalkyl.

In some cases, Z is tetrahydropyran. In some cases, Z is a carboxyl or carboxylic acid. In some cases, Z is 5-(2-Oxohexahydro-1H-thieno[3,4-d]imidazol-4-yl)pentanoic acid. In some cases, Z is 5-[(3AS,4R,6AR)-2-Oxohexahydro-1H-thieno[3,4-D]imidazol-4-YL]pentanoic acid.

In some cases, Z is pentafluorophenyl ester. For example, pentafluorophenyl ester has a structure of

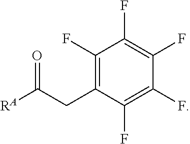

In some cases, R^{A} is selected from C₁-C₈ alkyl, for example, methyl or ethyl.

In some cases, Z is amide. In some cases, Z is alkyl, for example, saturated, straight-chain, or branched C₁-C₁₅ alkyl. In some cases, Z is methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1,1-dimethylpropyl, 1-ethylpropyl, hexyl, 1,1-dimethylbutyl, 2,2-dimethylbutyl, 3,3-dimethylbutyl, 1,2-dimethylbutyl, 1,3-dimethylbutyl, 2,3-dimethylbutyl, 1-methylpentyl, 2-methylpentyl, 3-methylpentyl, 4-methylpentyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl, or octyl.

In some cases, Z is N-substituted maleimide. For example, N-substituted maleimide has a structure of

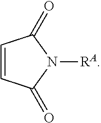

In some cases, R^{A} is selected from C₁-C₈ alkyl, halogen, amino, C₁-C₈ alkyloxy, cyano, nitro, aryl, heteroaryl, or C₃-C₈ cycloalkyl. In some cases, R^{A} is ethyl.
Siglec ligands of formula (XI) and conjugates of formula (XII)

Also provided by the present disclosure are Siglec ligands of formula (XI):

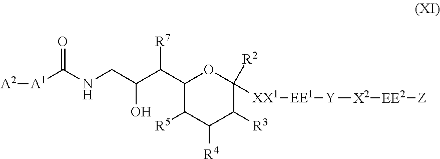

wherein:
A¹ is absent or alkylene;
A² is absent, alkyl, aryl, heteroaryl, cycloalkyl, or a substituted version thereof;
R² is —COOH, —P(O)(OH)₂, —OSO₂OH, —C(O)NHSO₂H, tetrazole, an ester, or a substituted version thereof;
R³ is H, alkyl, alkoxy, —S(alkyl), amino, halo, cyano, ester, amide, or a substituted version thereof;
R⁴ is hydroxy, alkoxy, amino, amide, or a substituted version thereof;
R⁵ is H, amino, ureido, amide, thioamide, oxamide, —NH(cyclobut-3-ene-1,2-dione), sulfonaamide, phosphoramide, alkylamine, triazole, tetrazole, or substituted version thereof;
R⁷ is hydroxy or halo;
XX¹ is S, triazole, —CH(OH)—, —CH₂—, —CHF—, —NH—, or —CF₂—;
EE¹ is absent, —(CH₂CH₂O)_{f}—, —(CH₂)_{g}—, benzyl, cycloalkyl, or a substituted version thereof, wherein f and g are each independently an integer ranging from 1 to 10;
Y is absent, alkylene, phenylene, substituted phenylene, —O—, —NH—, —S—, —C≡C—, —CH₂C≡C—, —C(O)—, —OP(O)O—, —NH-(cyclobut-3-ene-1,2-dione)-NH—, —NHC(O)—, triazole, —(CH₂CH₂O)ₘ—, —(CH₂)ₘ—, wherein m is an integer ranging from 1 to 10;
X² is absent, O, —NH—, —C(O)NH—, —C(O)—, —C(O)O—, —NHC(O)—, —NHC(O)O—, —OP(O)(OH)O—, —NHC(O)NH—, —(CH₂)ₙ—, —(CH₂CH₂O)ₙ—, —(CH₂CH₂O)ₙNHC(O)—, triazole, or a substituted version thereof, wherein n is an integer ranging from 1 to 10;
EE² is absent, —(CH₂)ₕ—, —(CH₂CH₂O)ₕC(O)NH—, —(CH₂CH₂O)ₕNHC(O)—, -(alkylene)C(O)NH(alkylene)-, (alkylene)C(O)NH(substituted alkylene)-, -(alkylene)C(O)N(substituted alkylene)₂-, —C≡C—, —CH₂C≡C—, or —(CH₂CH₂O)ᵢ—, wherein h and i are each independently selected from an integer ranging from 1 to 10; and
Z is Z1 or Z1′-T-Z2, wherein Z1 is a chemoselective functional group, wherein Z1′ is a diradical group, T is absent or a diradical group, and Z2 is a chemoselective functional group,
a salt thereof, or a stereoisomer thereof.

The XX¹ group of formula (XI) is S, triazole, —CH(OH)—, —CHF—, or —CF₂—. As such, in the Siglec ligands of formula (XI), the XX¹ group is not O. In contrast, in formula (I) above, the X¹ group could be O, S, triazole, —CH(OH)—, —CHF—, or —CF₂—. In some cases, the diradical group Z′, when starting from the connection to EE², can be an alkyl, substituted alkyl, polyethylene glycol, amino, alkoxy, or substituted alkoxy.

In the Siglec ligands of formula (XI), the EE¹ and EE² groups include the options of —(CH₂CH₂O)_{f}— and —(CH₂CH₂O)ᵢ—, respectively. In contrast, the Siglec ligands of formula (I) did not include the options of —(CH₂CH₂O)_{f}— and —(CH₂CH₂O)ᵢ— for E¹ and E².

In the terminology of formula (XI), the “linker” referred to above is the “XX¹-EE¹-Y—X²-EE²” moiety. As such, the “XX¹-EE¹-Y—X²-EE²” group can have advantageous properties that improve the technical qualities of a conjugate.

In some embodiments of formula (XI), the Siglec ligand has:
A¹ is absent or alkylene;
A² is absent, aryl, heteroaryl, cycloalkyl, or a substituted version thereof;
R² is —COOH, —P(O)(OH)₂, —OSO₂OH, —C(O)NHSO₂H, —C(O)NHSO₂R³—, tetrazole, or an ester thereof;
R³ is H, alkoxy, amino, or a substituted version thereof;
R⁴ is hydroxy, alkoxy, amino, amide, or a substituted version thereof;
R⁵ is amino, ureido, amide, thioamide, oxamide, —NH(cyclobut-3-ene-1,2-dione), sulfonamide or substituted version thereof;
R⁷ is hydroxy or halo;
XX¹ is S, triazole;
EE¹ is absent, —(CH₂CH₂O)_{f}—, or —(CH₂)_{g}—, or a substituted version thereof, wherein f and g are each independently an integer ranging from 1 to 10;
Y is alkylene, phenylene, or substituted phenylene;
X² is absent or O; and
EE² is absent, —(CH₂)ₕ—, -(alkylene)C(O)NH(alkylene)-, (alkylene)C(O)NH(substituted alkylene)-, -(alkylene)C(O)N(substituted alkylene)₂-, or —(CH₂CH₂O)ᵢ—, wherein h and i are each independently selected from an integer ranging from 1 to 10.

In some embodiments of formula (XI), the Siglec ligand has:
A¹ is absent;
A² is alkyl;
R² is —COOH;
R³ is H;
R⁴ is hydroxy;
R⁵ is ureido;
R⁷ is hydroxy.

In some embodiments of formula (XI), the Siglec ligand has:
A¹ is absent;
A² is substituted alkyl;
R² is tetrazole;
R³ is H;
R⁴ is hydroxy;
R⁵ is amide;
R⁷ is hydroxy.

In some embodiments of formula (XI), the Siglec ligand has:
A¹ is absent;
A² is substituted alkyl;
R² is —C(O)NHSO₂H;
R³ is H;
R⁴ is hydroxy;
R⁵ is oxamide;
R⁷ is hydroxy.

In some embodiments, the Siglec ligands of the formula (XI) are those of the formulae (XIa)-(XIe), where the symbols have the definitions and preferences indicated in the formula (XI).

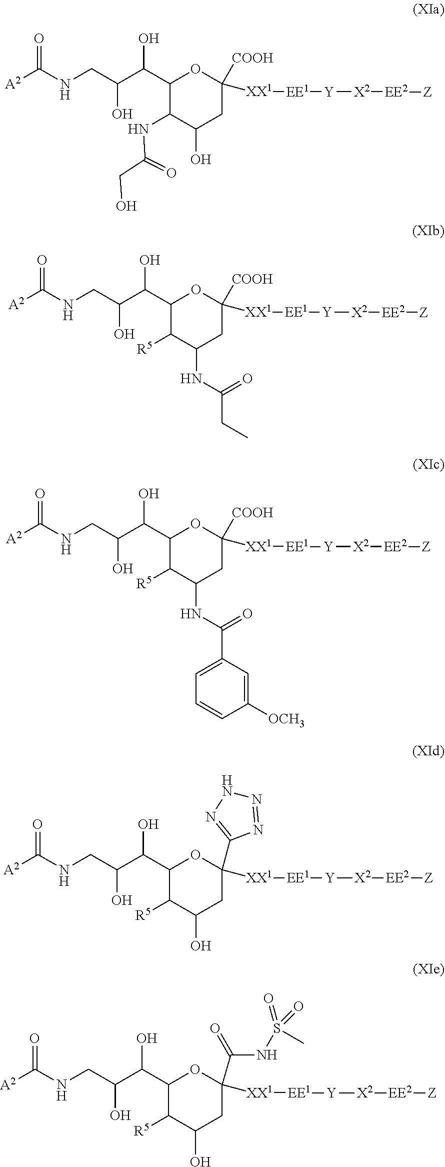

Also provided by the present disclosure are conjugates that include a Siglec ligand of formula (XI) and a biologically active substance. Provided are conjugates of formula (XII):

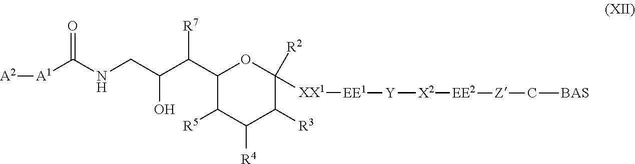

wherein:
A¹ is absent or alkylene;
A² is absent, alkyl, aryl, heteroaryl, cycloalkyl, or a substituted version thereof;
R² is —COOH, —P(O)(OH)₂, —OSO₂OH, —C(O)NHSO₂H, tetrazole, an ester, or a substituted version thereof;
R³ is H, alkyl, alkoxy, —S(alkyl), amino, halo, cyano, ester, amide, or a substituted version thereof;
R⁴ is hydroxy, alkoxy, amino, amide, or a substituted version thereof;
R⁵ is H, amino, ureido, amide, thioamide, oxamide, —NH(cyclobut-3-ene-1,2-dione), sulfonaamide, phosphoramide, alkylamine, triazole, tetrazole, or substituted version thereof;
R⁷ is hydroxy or halo;
XX¹ is S, triazole, —CH(OH)—, —CHF—, —CH₂—, —NH—, or —CF₂—;
EE¹ is absent, —(CH₂CH₂O)_{f}—, —(CH₂)_{g}—, benzyl, cycloalkyl, or a substituted version thereof, wherein f and g are each independently an integer ranging from 1 to 10;
Y is absent, alkylene, phenylene, substituted phenylene, —O—, —NH—, —S—, —C≡C—, —CH₂C≡C—, —C(O)—, —OP(O)O—, —NH-(cyclobut-3-ene-1,2-dione)-NH—, —NHC(O)—, triazole, —(CH₂CH₂O)ₘ—, —(CH₂)ₘ—, wherein m is an integer ranging from 1 to 10;
X² is absent, O, —NH—, —C(O)NH—, —C(O)—, —C(O)O—, —NHC(O)—, —NHC(O)O—, —OP(O)(OH)O—, —NHC(O)NH—, —(CH₂)ₙ—, —(CH₂CH₂O)ₙ—, —(CH₂CH₂O)ₙNHC(O)—, triazole, or a substituted version thereof, wherein n is an integer ranging from 1 to 10;
EE² is absent, —(CH₂)ₕ—, —(CH₂CH₂O)ₕC(O)NH—, —(CH₂CH₂O)ₕNHC(O)—, -(alkylene)C(O)NH(alkylene)-, (alkylene)C(O)NH(substituted alkylene)-, -(alkylene)C(O)N(substituted alkylene)₂-, —C≡C—, —CH₂C≡C—, or —(CH₂CH₂O)ᵢ—, wherein h and i are each independently selected from an integer ranging from 1 to 10; and
Z′ is Z1′ or Z1′-T-Z2′, wherein Z1′ is a diradical group, T is absent or a diradical group,
and Z2′ is a diradical group;
C is absent or a diradical group; and
BAS is a biologically active substance,
a salt thereof, or a stereoisomer thereof.

In some embodiments of formula (XII), the Siglec ligand has:
A¹ is absent or alkylene;
A² is absent, aryl, heteroaryl, cycloalkyl, or a substituted version thereof;
R² is —COOH, —P(O)(OH)₂, —OSO₂OH, —C(O)NHSO₂H, —C(O)NHSO₂R³—, tetrazole, or an ester thereof;
R³ is H, alkoxy, amino, or a substituted version thereof;
R⁴ is hydroxy, alkoxy, amino, amide, or a substituted version thereof;
R⁵ is amino, ureido, amide, thioamide, oxamide, —NH(cyclobut-3-ene-1,2-dione), sulfonamide or substituted version thereof;
R⁷ is hydroxy or halo;
X¹ is S or triazole;
E¹ is absent, —(CH₂CH₂O)_{f}—, or —(CH₂)_{g}—, or a substituted version thereof, wherein f and g are each independently an integer ranging from 1 to 10;
Y is alkylene, phenylene, or substituted phenylene;
X² is absent or O; and
EE² is absent, —(CH₂)ₕ—, -(alkylene)C(O)NH(alkylene)-, (alkylene)C(O)NH(substituted alkylene)-, -(alkylene)C(O)N(substituted alkylene)₂-, or —(CH₂CH₂O)ᵢ—, wherein h and i are each independently selected from an integer ranging from 1 to 10.

In some embodiments of formula (XII), the Siglec conjugate has:
A¹ is absent;
A² is alkyl;
R² is —COOH;
R³ is H;
R⁴ is hydroxy;
R⁵ is ureido;
R⁷ is hydroxy.

In some embodiments of formula (XII), the Siglec conjugate has:
A¹ is absent;
A² is substituted alkyl;
R² is tetrazole;
R³ is H;
R⁴ is hydroxy;
R⁵ is amide;
R⁷ is hydroxy.

In some embodiments of formula (XII), the Siglec conjugate has:
A¹ is absent;
A² is substituted alkyl;
R² is —C(O)NHSO₂H;
R³ is H;
R⁴ is hydroxy;
R⁵ is oxamide;
R⁷ is hydroxy.

In some embodiments, the Siglec conjugates of the formula (XII) are those of the formulae (XIIa)-(XIIe), where the symbols have the definitions and preferences indicated in the formula (XII).

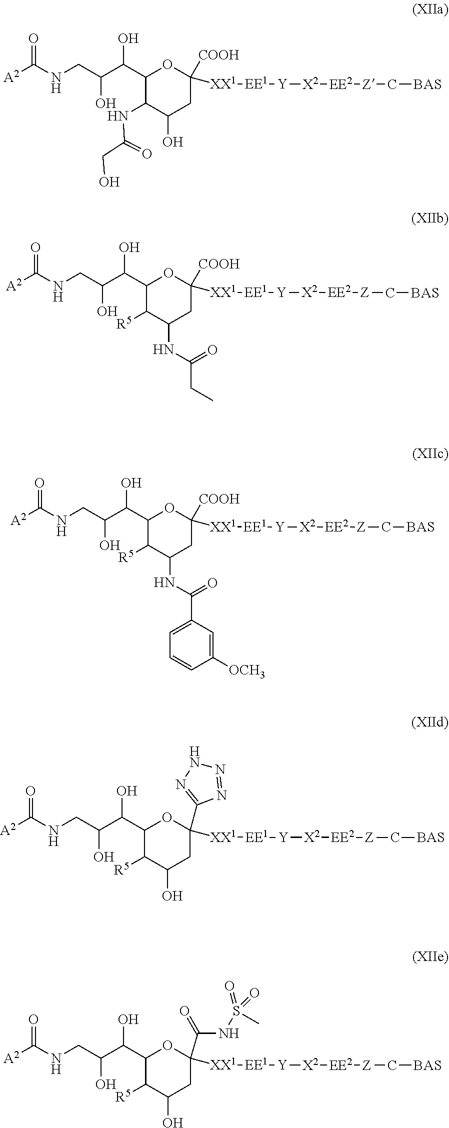

In some embodiments of formula (XI) and/or formula (XII), A¹ is absent. In some cases, A¹ is alkylene, for example, A¹ is ethylene.

In some embodiments of formula (XI) and/or formula (XII), A² is alkyl, for example, saturated, straight-chain, or branched C₁-C₁₅ alkyl. In some cases, A² is methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1,1-dimethylpropyl, 1-ethylpropyl, hexyl, 1,1-dimethylbutyl, 2,2-dimethylbutyl, 3,3-dimethylbutyl, 1,2-dimethylbutyl, 1,3-dimethylbutyl, 2,3-dimethylbutyl, 1-methylpentyl, 2-methylpentyl, 3-methylpentyl, 4-methylpentyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl, or octyl.

In some cases, A² is a substituted version of alkyl, for example substituted with C₁-C₈ alkyl, halogen, amino, C₁-C₈ alkyloxy, cyano, nitro, aryl, heteroaryl, or C₃-C₈ cycloalkyl.

In some embodiments of formula (XI) and/or formula (XII), A² is an aryl, for example, C₆-C₁₄ aryl. In some cases, A² is phenyl, 1-naphthyl, 2-naphthyl, 1-biphenylene, or 2-biphenylene. In some cases, A² is a substituted version of an aryl, for example substituted with C₁-C₈ alkyl, halogen, hydroxy, amino, C₁-C₈ alkyloxy, C₁-C₈ haloalkyl, cyano, nitro, aryl, heteroaryl, or C₃-C₈ cycloalkyl.

In some embodiments of formula (XI) and/or formula (XII), A² is a heteroaryl for example, 5-10 membered heteroaryl containing one or more heteroatoms selected from S, N, O or SO₂. In some cases, A² is furyl, thienyl, pyrrolyl, pyrazolyl, oxazolyl, pyridinyl, pyrimidinyl, pyrazinyl or 4H-thieno[3,2-c]chromene. In some cases, A² is a substituted version of a heteroaryl, for example substituted with C₁-C₈ alkyl, halogen, amino, C₁-C₈ alkyloxy, cyano, nitro, aryl, heteroaryl, or C₃-C₈ cycloalkyl.

In some embodiments of formula (XI) and/or formula (XII), A² is a cycloalkyl, for example, C₃-C₈ cycloalkyl. In some cases, A² is cyclopropyl, cyclobutyl, cyclopentyl, cyclohexyl, cycloheptyl or cyclooctyl.

In some cases, A¹ is absent and A² is —CR^{X}R^{Y}R^{Z}.

In some cases, R^{X} is H, F, —CF₃, —OH.

In some cases, R^{Y} is H, F, —OCH₃, —CH₃.

In some cases, R^{Z} is —CH₃. In some cases, R^{Z} is selected from any one of the groups described below:

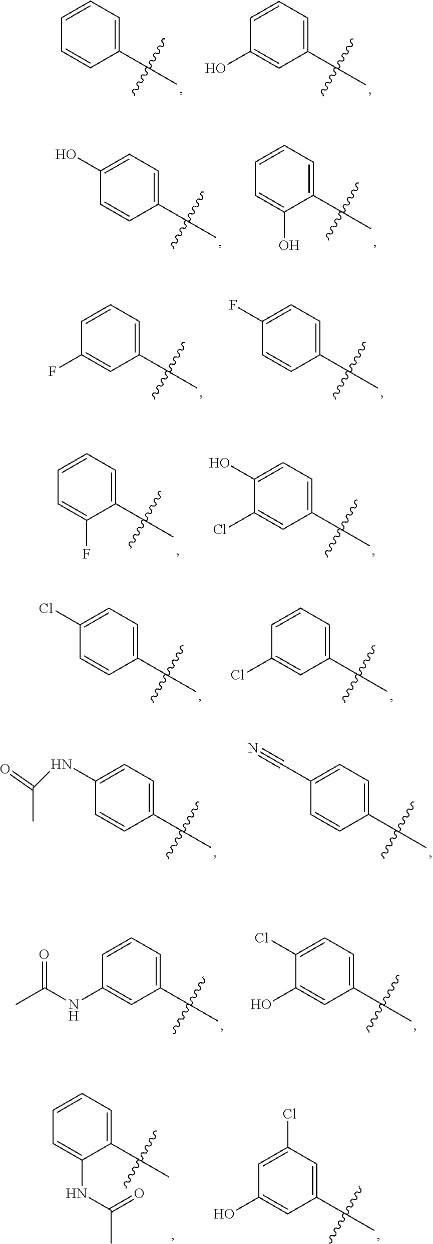

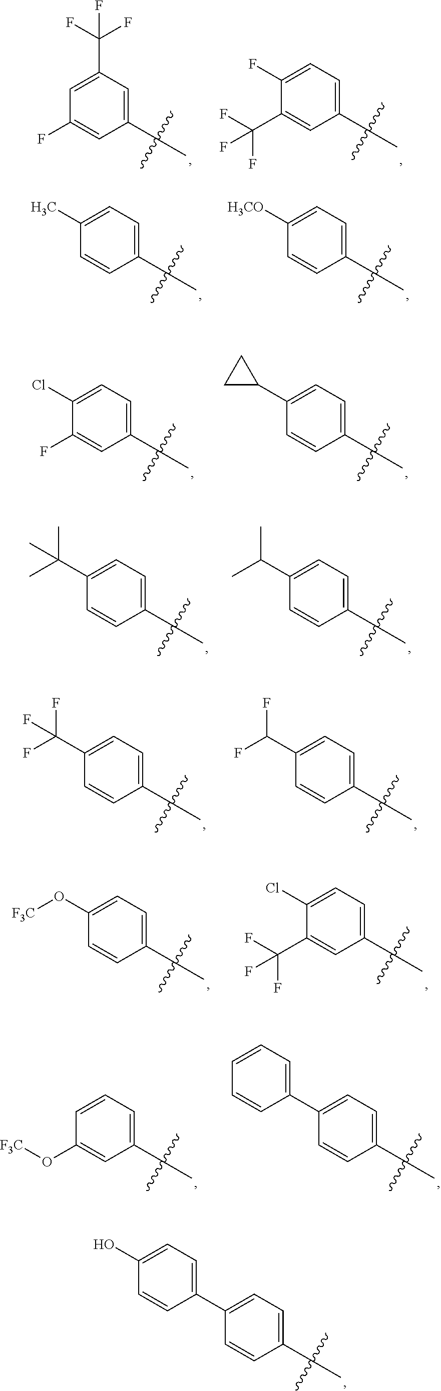

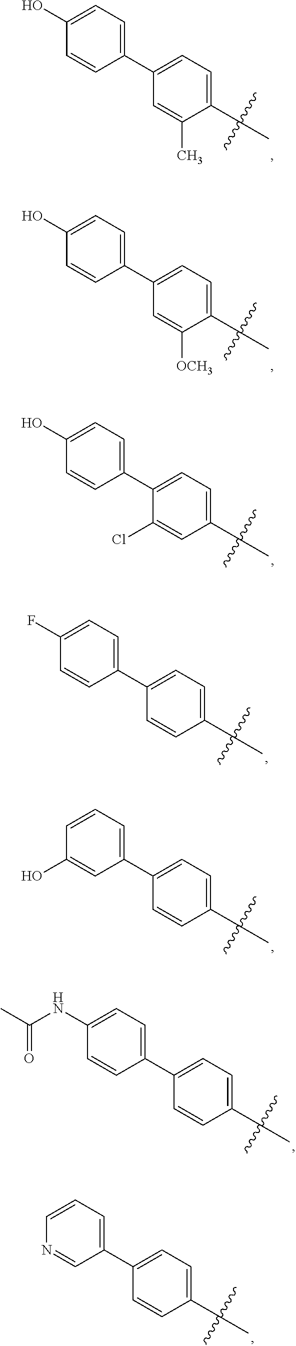

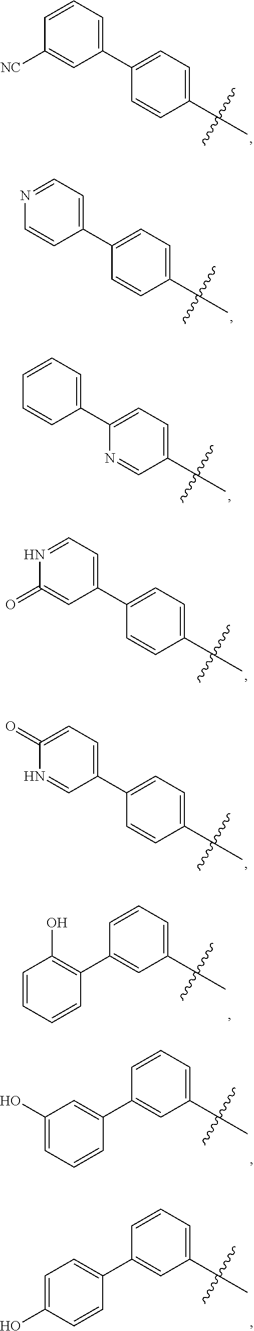

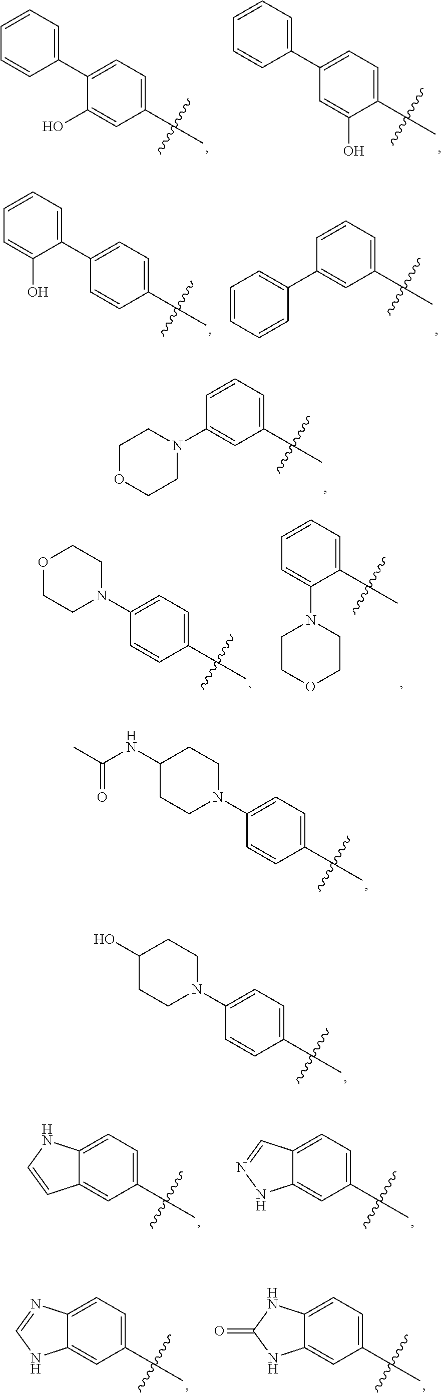

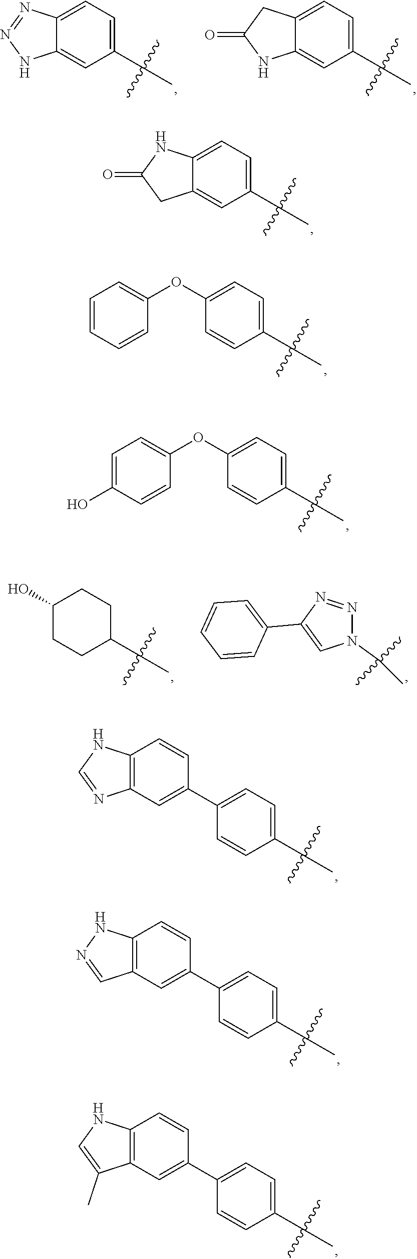

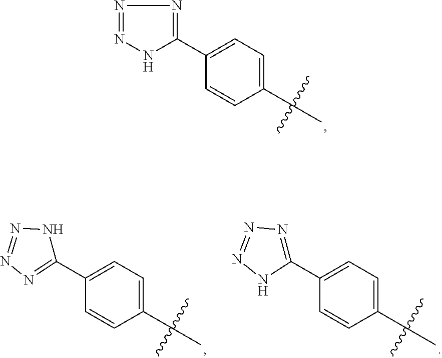 [SMILES: *c1ccc(-c2nnn[nH]2)cc1 ; *c1ccc(-c2nnn[nH]2)cc1 ; *c1ccc(-c2nnn[nH]2)cc1]

In some cases, R^{X} is H, R^{Y} is H and R^{Z} is selected from any of the groups described above.

In some cases, A¹ is absent and A² is cyclopropyl substituted with any one of the groups selected from the following:

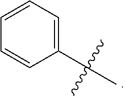 [SMILES: *c1ccccc1]

In some cases, A¹ is absent and A² is tetrahydropyran substituted with any one of the groups selected from the following:

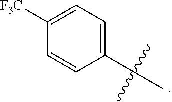 [SMILES: *c1ccc(C(F)(F)F)cc1]

In some embodiments of formula (XI) and/or formula (XII), R² is —COOH or a substituted version. In some cases, R² is —P(O)(OH)₂ or a substituted version. In some cases, R² is —OSO₂OH or a substituted version. In some cases, R² is —C(O)NHSO₂H or a substituted version. In some cases, R² is tetrazole or a substituted version. In some cases, R² is an ester or a substituted version.

In some embodiments of formula (XI) and/or formula (XII), R³ is H. In some cases, R³ is alkyl, for example, saturated, straight-chain, or branched C₁-C₈ alkyl. In some cases, R³ is alkoxy, for example, saturated, straight-chain, branched or cyclic C₁-C₈ alkoxy. In some cases, R³ is —S(alkyl), for example, —S(C₁-C₈ alkyl). In some cases, R³ is amino or a substituted version. In some cases, R³ is halo, for example fluorine, chlorine, bromine or iodine. In some cases, R³ is cyano. In some cases, R³ is ester or a substituted version. In some cases, R³ is amide or a substituted version.

In some embodiments of formula (XI) and/or formula (XII), R⁴ is hydroxy. In some cases, R⁴ is alkoxy, for example, saturated, straight-chain, branched or cyclic C₁-C₈ alkoxy. In some cases, R⁴ is amino. In some cases, R⁴ is amide or a substituted version.

In some cases, R⁴ is —NHC(O)CH₂CH₃. In some cases, R⁴ is —NHC(O)C₆H₄OCH₃.

In some embodiments of formula (XI) and/or formula (XII), R⁵ is amino or a substituted version. In some cases, R⁵ is ureido or a substituted version. In some cases, R⁵ is amide or a substituted version. In some cases, R⁵ is thioamide or a substituted version. In some cases, R⁵ is oxamide or a substituted version. In some cases, R⁵ is —NH(cyclobut-3-ene-1,2-dione) or a substituted version. In some cases, R⁵ is sulfonamide or a substituted version. In some cases, R⁵ is phosphoramide or a substituted version. In some cases, R⁵ is alkylamine or a substituted version. In some cases, R⁵ is triazole or a substituted version. In some cases, R⁵ is tetrazole or a substituted version.

In some embodiments, R⁵ is —NHC(O)CR^{A}R^{B}R^{C}, —NHC(O)NR^{A}R^{B}, —NHC(O)C(O)NR^{A}R^{B}, —NHC(S)CR^{A}R^{B}R^{C}, —NHC(S)NR^{A}R^{B}, —NR^{A}R^{B}, —NHS(O)₂NR^{A}R^{B}.

In some cases, R^{A} is H, —CH₃, F.

In some cases, R^{B} is H, —CH₃, —C₆H₅, F, —CH₂CF₃, —CH₂CH₂OH, cyclopropyl methyl, —OH, cyclopropyl, —CH₂CH₃, —CH₂C(O)NH₂, —CH₂C(O)NHCH₃, —CH₂C(O)NCH₃CH₃.

In some cases, R^{C} is —OH, F, H, —SH, —OCH₃, —CN, —NHC(O)CH₃, —C₆H₅, —CH₂OH, —NH₂, —NHCH₃, —NCH₃CH₃, —OC₆H₅, —CH₃, tetrazole.

In some embodiments, R⁵ is —NHC(O)C₆H₅.

In some embodiments of formula (XI) and/or formula (XII), R⁷ is hydroxy. In some cases, R⁷ is halo, for example fluorine, chlorine, bromine or iodine.

In some embodiments of formula (XI) and/or formula (XII), XX¹ is S. In some cases, XX¹ is triazole. In some cases, XX¹ is —CH(OH)—. In some cases, XX¹ is —CH₂—. In some cases, XX¹ is —CHF—. In some cases, XX¹ is —CF₂—. In some cases, XX¹ is —NH—.

In some embodiments of formula (XI) and/or formula (XII), EE¹ is absent. In some cases, EE¹ is —(CH₂CH₂O)_{f}— or a substituted version. In some cases, EE¹ is —(CH₂)_{g}— or a substituted version. In some cases, f is an integer ranging from 1 to 10. In some cases, g is an integer ranging from 1 to 10. In some cases, EE¹ is benzyl or a substituted version. In some cases, EE¹ is cycloalkyl or a substituted version. For example, EE¹ is C₃-C₈ cycloalkyl. In some cases, EE¹ is cyclopropyl, cyclobutyl, cyclopentyl, cyclohexyl, cycloheptyl or cyclooctyl.

In some embodiments of formula (XI) and/or formula (XII), Y is absent. In some cases, Y is alkylene, for example ethylene. In some cases, Y is phenylene. In some cases, Y is —O—. In some cases, Y is —S—. In some cases, Y is —NH—. In some cases, Y is a substituted phenylene. In some cases, Y is —C≡C—. In some cases, Y is —CH₂C≡C—. In some cases, Y is —C(O)—. In some cases, Y is —OP(O)O—. In some cases, Y is —NH-(cyclobut-3-ene-1,2-dione)-NH—. In some cases, Y is —NHC(O)—. In some cases, Y is a triazole. In some cases, Y is —(CH₂CH₂O)ₘ—. In some cases, Y is —(CH₂)ₘ—. In some cases, Y is m is an integer ranging from 1 to 10.

In some embodiments of formula (XI) and/or formula (XII), XX² is absent. In some cases, XX² is O. In some cases, XX² is —NH—. In some cases, XX² is —C(O)NH—. In some cases, XX² is —NHC(O)—. In some cases, XX² is —NHC(O)O—. In some cases, XX² is —OP(O)(OH)O—. In some cases, XX² is —NHC(O)NH—. In some cases, XX² is —(CH₂)ₙ—. In some cases, XX² is —(CH₂CH₂O)ₙ—. In some cases, XX² is —(CH₂CH₂O)ₙNHC(O)—. In some cases, XX² is a triazole. In some cases, XX² is n is an integer ranging from 1 to 10. In some cases, XX² is —C(O)—. In some cases, XX² is —C(O)O—.

In some embodiments of formula (XI) and/or formula (XII), EE² is absent. In some cases, EE² is —(CH₂)ₕ—. In some cases, EE² is —(CH₂CH₂O)ᵢ—. In some cases, EE² is —(CH₂CH₂O)ₕC(O)NH—. In some cases, EE² is —(CH₂CH₂O)ₕNHC(O)—. In some cases, EE² is -(alkylene)C(O)NH(alkylene)-. In some cases, EE² is (alkylene)C(O)NH(substituted alkylene)-. In some cases, EE² is -(alkylene)C(O)N(substituted alkylene)₂-. In some cases, EE² is —C≡C—. In some cases, EE² is —CH₂C≡C—. In some cases, h is an integer ranging from 1 to 10. In some cases, i is an integer ranging from 1 to 10.

In some embodiments of formula (XI) and/or formula (XII), Z is selected from a group consisting of alkyne, azide, thiol, maleimide, iodoacetamide, amine, carboxylic acid or active ester thereof, tetrazine, trans-cyclooctene, diene, dienophile, hydroxyl, hydrazido, hydrazino, aldehyde, ketone, azido, phosphine, epoxide, succinimide, and phosphate.

In some embodiments of formula (XI) and/or formula (XII), Z is C₂-C₁₀ alkyne, for example, ethyne, propyne, butyne, pentyne, hexyne, heptyne, nonyne or decyne. In some cases, Z is an aryl, for example, C₆-C₁₄ aryl. In some cases, Z is phenyl, 1-naphthyl, 2-naphthyl, 1-biphenylene, or 2-biphenylene. In some cases, Z is a substituted version of an aryl, for example substituted with C₁-C₈ alkyl, halogen, hydroxy, amino, C₁-C₈ alkyloxy, C₁-C₈ haloalkyl, cyano, nitro, aryl, heteroaryl, or C₃-C₈ cycloalkyl.

In some cases, Z is tetrahydropyran. In some cases, Z is a carboxyl or carboxylic acid. In some cases, Z is 5-(2-Oxohexahydro-1H-thieno[3,4-d]imidazol-4-yl)pentanoic acid. In some cases, Z is 5-[(3AS,4R,6AR)-2-Oxohexahydro-1H-thieno[3,4-D]imidazol-4-YL]pentanoic acid.

In some cases, Z is pentafluorophenyl ester. For example, pentafluorophenyl ester has a structure of

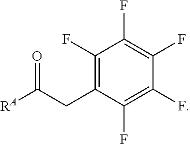

In some cases, R^{A} is selected from C₁-C₈ alkyl, for example, methyl or ethyl.

In some cases, Z is amide. In some cases, Z is alkyl, for example, saturated, straight-chain, or branched C₁-C₁₅ alkyl. In some cases, Z is methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1,1-dimethylpropyl, 1-ethylpropyl, hexyl, 1,1-dimethylbutyl, 2,2-dimethylbutyl, 3,3-dimethylbutyl, 1,2-dimethylbutyl, 1,3-dimethylbutyl, 2,3-dimethylbutyl, 1-methylpentyl, 2-methylpentyl, 3-methylpentyl, 4-methylpentyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl, or octyl.

In some cases, Z is N-substituted maleimide. For example, N-substituted maleimide has a structure of

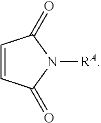

In some cases, R^{A} is selected from C₁-C₈ alkyl, halogen, amino, C₁-C₈ alkyloxy, cyano, nitro, aryl, heteroaryl, or C₃-C₈ cycloalkyl. In some cases, R^{A} is ethyl.

### Aspects of Siglec Ligands and Conjugates Thereof

As discussed above, the conjugates provided herein include a Siglec ligand and biologically active substance (BAS). As used herein, the term “biologically active substance” refers to a substance that causes a change in a biological system when the biological system is contacted with the substance. In some cases, the biologically active substance is a protein. In some cases, the biologically active substance is a biotherapeutic. In some cases, the biologically active substance is an autoantigen.

### Biotherapeutic

The term “biotherapeutic” refers to a composition that is composed of sugars, amino acids, proteins, lipids or nucleic acids or complex combinations of these substances and that is therapeutic in an individual. Examples of biotherapeutics include protein therapeutics (e.g. antibodies, fusion proteins, and enzymes), viral therapeutics (e.g. viral particle), cell therapeutics, and nucleic acid therapeutics (e.g. polypeptides and nucleic acids)

Any protein or nucleic acid biotherapeutic may serve as the biotherapeutic that is engineered to become a hypoimmunogenic biotherapeutic according to the present disclosure, including, for example, a protein, e.g. an antibody, a fusion protein, an enzyme, a viral particle, a DNA molecule or an RNA molecule. The biotherapeutic may be naturally occurring, for example a naturally occurring protein that is delivered to a patient as a therapeutic, a naturally occurring capsid, etc. The biotherapeutic may be an engineered protein, for example, an antibody therapeutic, a fusion or “chimeric” protein, i.e. a protein comprising protein domains from two or more different proteins, or an entirely non-natural protein, i.e. having 30% identity or less with any naturally occurring protein across its functional domains (see e.g. Chen et al. (2020) De novo design of protein logic gates. Science 368 (6486): 78-84; and Polizzi et al. (2020) A defined structural unit enables de novo design of small-molecule-binding proteins Science 369 (6508): 1227-1233). In some embodiments, the biotherapeutic is a variant of a naturally occurring protein or a known engineered protein. By “variant” it is meant a mutant of a protein having less than 100% sequence identity with the protein from which it is derived. For example, a variant protein may be a protein having 60% sequence identity or more with a full length native protein, e.g. 65%, 70%, 75%, or 80% or more identity, such as 85%, 90%, or 95% or more identity, for example, 98% or 99% identity with the full length native protein. Variants also include fragments of naturally occurring proteins, particularly those having comparable or improved activity over the naturally occurring protein. The biotherapeutic may be derived from any source, e.g. human, non-human, or engineered.

Of particular interest is the suppression of an immune response to a biotherapeutic, and more particularly, of a B cell response to the biotherapeutic. Accordingly, in some embodiments, the Siglec ligand is a ligand for a Siglec that is expressed on B lymphocytes, for example Siglec-2 (also called CD22), Siglec-5 (CD170), Siglec-6, Siglec-9 (CD329), or Siglec-10 (Siglec-G). In some embodiments, the Siglec is Siglec-2. In some embodiments, the Siglec is Siglec-5. In some embodiments, the Siglec is Siglec-6. In some embodiments, the Siglec is Siglec-9. In some embodiments, the Siglec is Siglec-10. In some embodiments, the hypoimmunogenic biotherapeutic has been engineered to comprise the sialic acid ligands for one Siglec. In other embodiments, the hypoimmunogenic biotherapeutic has been engineered to comprise the Siglec ligands for two or more Siglecs, e.g. for 3 Siglecs or for 4 Siglecs, in certain cases, for 5 Siglecs. In some cases there are 5, 6, 7, 8, 9, 10, 11, 12, 13, or 15 Siglecs.

In some embodiments, the protein is an antibody or fragment thereof, for example a monoclonal antibody, a bispecific antibody, a trispecific antibody, an scFv, a Fab, a camelid nanobody, etc. Nonlimiting examples of antibodies for which the engineering contemplated herein finds particular use include adalimumab and infliximab (for the treatment of autoimmune or an inflammatory disease such as rheumatoid arthritis, psoriatic arthritis, ankylosing spondylitis, Crohn's disease, ulcerative colitis, psoriasis, hidradenitis suppurativa, uveitis, or juvenile idiopathic arthritis), cetuximab (for the treatment of cancers, including for example metastatic colorectal cancer, metastatic non-small cell lung cancer and head and neck cancer), natalizumab (for the treatment of multiple sclerosis), Lumoxiti/moxetumomab pasudotox (for the treatment of hairy cell leukemia), Tecentriq/atezolizumab (for the treatment of various cancers), Opdivo/Nivolumab (for the treatment of various cancers), Reopro/abciximab (anti-GPIIb/IIIa, for the prevention of thrombosis during and after coronary artery procedures such as angioplasty), Brentuximab (for the treatment of relapsed or refractory Hodgkin lymphoma (HL) and systemic anaplastic large cell lymphoma (ALCL)), Certolizumab pegol (for the treatment of Crohn's disease, rheumatoid arthritis, psoriatic arthritis and ankylosing spondylitis), Elotuzumab (for the treatment of relapsed multiple myeloma), Benralizumab (for the treatment of asthma), Vedolizumab (for the treatment of ulcerative colitis and Crohn's disease), Galcanezumab (for the treatment of migraines and cluster headaches), Rituximab (for the treatment of autoimmune diseases and various cancer), Alemtuzumab (for the treatment of chronic lymphocytic leukemia (CLL) and multiple sclerosis), Dupilumab (for the treatment of allergic diseases such as eczema (atopic dermatitis), asthma and nasal polyps), Golimumab (for the treatment of inflammation), Obinutuzumab (for the treatment of lymphomas, e.g. chronic lymphocytic leukemia, follicular lymphoma), Tildrakizumab (for the treatment of immunologically mediated inflammatory disorders), Erenumab (for the prevention of migraine), Mepolizumab (for the treatment of severe eosinophilic asthma, eosinophilic granulomatosis, and hypereosinophilic syndrome (HES)), Ramucirumab (for the treatment of solid tumors), Ranibizumab (for the treatment of “wet” age-related macular degeneration (AMD, also ARMD), diabetic retinopathy, and macular edema), Ustekinumab (for the treatment of psoriasis, Crohn's disease, and ulcerative colitis), Reslizumab (for the treatment of asthma), Ipilimumab (for the treatment of various cancers), Alirocumab (for the treatment of high cholesterol), Belimumab (for the treatment of systemic lupus erythematosus (SLE)), Panitumumab (for the treatment of various cancers), Avelumab (for the treatment of Merkel cell carcinoma, urothelial carcinoma, and renal cell carcinoma), Necitumumab (for the treatment of metastatic squamous non-small-cell lung carcinoma (NSCLC)), Mogamulizumab (for the treatment of relapsed or refractory mycosis fungoides and Sézary disease, relapsed or refractory CCR4+ adult T-cell leukemia/lymphoma (ATCLL), and relapsed or refractory CCR4+ cutaneous T cell lymphoma (CTCL)), Olaratumab (for the treatment of solid tumors), Brodalumab (for the treatment of inflammatory diseases), Eculizumab (for the treatment of paroxysmal nocturnal hemoglobinuria (PNH), atypical hemolytic uremic syndrome (aHUS), and neuromyelitis optica), Pertuzumab (for the treatment of metastatic HER2-positive breast cancer), Pembrolizumab (for the treatment of various cancers), and Tocilizumab (for the treatment of rheumatoid arthritis (RA) and systemic juvenile idiopathic arthritis)

In some embodiments, biotherapeutic is an antibody that is not an antibody that is specific for a receptor selected from a B cell receptor (BCR), a receptor for the Fc region of immunoglobulin E (FcIRI), a Toll Like receptor (TLR), a T-cell receptor (TCR), or complexes thereof.

As used herein, an antibody that specifically binds to a target antigen refers to an antibody comprising a complementarity determining region (CDR) domain that specifically recognizes and binds to the target antigen. Thus, an antibody that specifically binds to a B cell receptor or complex thereof refers to an antibody comprising a CDR that specifically recognizes and binds to a B cell receptor or a complex comprising a B cell receptor, an antibody that specifically binds to a receptor for the Fc region of IgE refers to an antibody comprising a CDR that specifically recognizes and binds to a receptor for the Fc region of IgE or a complex comprising a receptor for the Fc region of IgE, an antibody that specifically binds to a Toll like receptor refers to an antibody comprising a CDR that specifically recognizes and binds to a Toll-like receptor or a complex comprising a Toll-like receptor, and an antibody that specifically binds to a T-cell receptor refers to an antibody comprising a CDR that specifically recognizes and binds to a T-cell receptor or a complex comprising a T-cell receptor

In some embodiments, the protein is a native, or naturally occurring, protein. In other embodiments, the protein is an engineered protein. Examples of proteins for which the engineering contemplated herein finds particular use include erythropoietin (EPO, to stimulate the production of red blood cells), thrombopoietin (TPO, to stimulate the production of platelets), human growth hormone, tissue factor, IFNβ-1b (for the treatment of Multiple Sclerosis), IFNβ-1a (for the treatment of Multiple Sclerosis), IL-2 or the IL-2 mimetic aldesleukin (for the treatment of melanoma and renal cell carcinoma), exenatide (for the treatment of Type 2 Diabetes), albiglutide (for the treatment of Type 2 Diabetes), alefacept (to control inflammation in moderate to severe psoriasis with plaque formation, for the treatment of cutaneous T-cell lymphoma and T-cell non-Hodgkin lymphoma), palifermin (to stimulate the growth of cells that line the surface of the mouth and intestinal tract following chemotherapy), belatacept (to promote graft/transplant survival), and neutral and basic amino acid transport protein rBAT or b(0,+)-type amino acid transporter 1 (for the treatment of cystinuria)

In some embodiments, biotherapeutic is a protein that is not ovalbumin or immunoglobulin E (IgE)

In some embodiments, the protein is an enzyme, for example a metabolic enzyme, a lysosomal enzyme, a protease, a peptidase, etc. Nonlimiting examples of enzymes for which the engineering contemplated herein finds particular use include asparaginase from Erwinia chrysanthemi (for the treatment of leukemia), bacterial IdeS (for immunosuppression following tissue transplantation or in the administration of a therapy, e.g. a gene therapy, for which the patient had preexisting immunity; for treatment of IgG antibody-driven diseases, such as Systemic lupus erythematosus, Pemphigus vulgaris or IgA Nephropathy), bacterial mucinase (for the treatment of MUC+ cancers, e.g. MUC1+ cancers), Factor VIII (for the treatment of Hemophilia A), Factor IX (for the treatment of Hemophilia B), Factor Xa (to promote clotting), a complement degrading protease, e.g. from a pathogen such as a bacterial pathogen or fungal pathogen (e.g. Pseudomonas Elastase (PaE), Pseudomonas Alkaline protease (PaAP), Streptococcal pyrogenic Exotoxin B (SpeB), a gingipain from Porphyromonas gingivalis, Aspergillus Alkaline protease 1 (Alp1), C. albicans Secreted aspartyl proteinases 1 (Sap1), 2 (Sap2), and 3 (Sap3) for the treatment of complement-mediated disease, such as IgA nephropathy), phenylalanine ammonia-lyase or the mimetic pegvaliase (for the treatment of PKU), alpha-galactosidase A (for the treatment of Fabry Disease), acid α-glucosidase or the mimetic Alglucosidase alfa (GAA, for the treatment of Pompe Disease), glucocerebrosidase (GCase, for the treatment of Gaucher), aspartylglucosaminidase (AGA, for the treatment of Aspartylglucosaminuria), asfotase (for treatment of hypophosphatasia (HPP)), alpha-L-iduronidase (for the treatment of MPS I), iduronate sulfatase or the iduronate sulfatase mimetic idursulfase (for the treatment of MPS II), sulfaminase (for the treatment of MPS IIIa), α-N-acetylglucosaminidase (NAGLU, for the treatment of MPS IIIB), heparin acetyle CoA: α-glucosaminide N-acetyltransferase (HGSNAT, for the treatment of MPS IIIC), N-acetylglucosamine 6-sulfatase (GNS, for the treatment of MPS IIID), N-glucosamine 3-O-sulfatase (arylsulfatase G or ARSG, for the treatment of MPS IIIE), N-acetylgalactosamine 6-sulfatase (for the treatment of MPS IVA), beta-galactosidase (for the treatment of MPS IVB), N-acetylgalactosamine 4-sulfatase (for the treatment of MPS VI), beta-glucuronidase (for the treatment of MPS VI), palmitoyl protein thioesterase (PPT1, for the treatment of Batten disease/CLN1), Tripeptidyl peptidase (TPP1, for the treatment of Batten Disease/CLN2), arginase-1 or pegzilarginase (for the treatment of arginase-1 deficiency), or cystathionine beta synthase or Aeglea product AGLE-177 (for the treatment of cystathionine beta synthase (CBS) deficiency, also known as Classical Homocystinuria)

In some embodiments, biotherapeutic is an enzyme that is not Factor VIII.

In some embodiments, the protein is a viral protein or a viral particle, for example, a recombinant viral particle. By a “recombinant” virus or viral particle it is meant a virus/viral particle that comprises a genome comprising a polynucleotide that is heterologous to the virus, i.e., not found in nature to be associated with the capsid/envelope of the virus, wherein the polynucleotide encodes a gene product (RNA or protein). Recombinant viral particles find use in the delivery of polynucleotides that encode a therapeutic gene product for the purpose of gene therapy or oncolytic virus therapy. Gene therapy is a well-established art. Also well-established is the fact that gene therapy is severely hampered by the inability to readminister the same viral therapeutic more than once or a few times, owing to the fact that the viral particle will induce an immune response in an individual. As such, the ordinarily skilled artisan will appreciate that any viral particle used in gene therapy would benefit from engineering as contemplated herein. Nonlimiting examples of viral particles that may serve as the biotherapeutic that is engineered to become a hypoimmunogenic biotherapeutic according to the present disclosure include recombinant adeno-associated virus (rAAV) particles, e.g. an rAAV particle comprising a capsid VP1 protein from the group consisting of an AAV1, AAV2, AAV3b, AAV4, AAV5, AAV6, AAV7, AAV8, AAV9, AAVrh10, AAV11, AAV12, or AAV13 VP1 protein or a variant or pseudotyped virus thereof; recombinant human adenovirus particles, e.g. an rHAdV particle comprising a capsid protein from rHAdV-A, rHAdV-B, rHAdV-C, rHAdV-D, rHAdV-E, rHAdV-F, or rHAdV-G or a variant thereof; recombinant Herpes Simplex Virus (rHSV) particles, e.g. a rHSV1 or rHSV2 or variant or pseudotyped virus thereof; recombinant papillomavirus (PV) particles; recombinant polyomavirus particles; recombinant vaccinia virus particles; a recombinant cytomegalovirus (CMV) particle; a recombinant baculovirus particle; a recombinant human papillomavirus (HPV) particle; or a recombinant retrovirus particle, e.g. a recombinant lentivirus, recombinant human immunodeficiency virus (HIV) particle, Simian immunodeficiency virus (SIV) particle, Feline immunodeficiency virus (FIV) particle, Puma lentivirus (PLV) particle, Equine infectious anemia virus (EIAV) particle, Bovine immunodeficiency virus (BIV) particle, Caprine arthritis encephalitis virus particle, gammaretrovirus particle, and murine leukemia virus (MLV) particle, or variant or pseudotyped virus thereof.

In some embodiments, biotherapeutic is not a toxin. Generally, toxins are compounds that are harmful to cells in generally non-specific manner, i.e. a toxin will cause a similar amount of harm to different cells, even if such cells are from significantly different categories. In contrast, selectively damaging compounds will harm certain cells to a significantly greater degree than the harm inflicted on other types of cells. For instance, the selectively damaging compound can cause harm based on a biochemical process that is common in a lung cell but rare in a kidney cell, whereas a toxin can cause harm based on a biochemical process common to both lung and kidney cells. In some cases, the harm is cell death. In some cases, the toxin is Pseudomonas exotoxin A. In some embodiments, biotherapeutic is not a B cell modulator. Y does not increase or decrease the immune action of a B cell. Examples of modulation of the B cell include differentiation of the B cell into a biotherapeutic-specific mature B cell, e.g. plasma cells or memory cells, preventing B cells from producing antigen-specific antibodies, preventing the upregulation of activation markers such as CD69, promoting a decrease in viability of a biotherapeutic-specific B cell population. In some embodiments, B cell activation is inhibited only for those B cells with a B cell receptor that recognizes Y (in contrast to the entire B cell population recognizing X).

### Autoantigen

The term “autoantigen” (abbreviated as “AutoAg”) refers to an endogenous molecule present in a subject which the subject's immune system does not recognize as an endogenous molecule (i.e., as a self-antigen) and thus mounts an immune response to the self-antigen. Such an immune response is referred to as an autoimmune response. The autoantigen is encoded by an endogenous gene present in a subject and may be a polypeptide, e.g., a soluble or membrane-localized polypeptide, a lipidated, glycosylated, or otherwise post-translationally modified polypeptide; a nucleic acid (e.g., DNA, RNA); or a complex thereof. In some embodiments, the autoantigen is any autoantigen that elicits a B-cell driven immune response in an individual, where the individual produces B cells that bind to the autoantigen via B-cell receptors (BCRs) and upon binding to the autoantigen differentiate into plasma cells that produce autoantibodies that bind to the autoantigen.

In contrast to a biotherapeutic, an autoantigen is a molecule that is endogenously produced by the subject, whereas a biotherapeutic is a molecule that the subject does not produce and is exogenously supplied to the person, e.g., as a polypeptide or a gene encoding the polypeptide. Thus, an immune response to a biotherapeutic is not considered an autoimmune response. Rather, an immune response to a biotherapeutic is a normal immune response. A biotherapeutic may be composed of sugars, amino acids, proteins, lipids or nucleic acids or complex combinations of these substances. Unlike an engineered autoantigen, a biotherapeutic is not intended as an autoimmune-suppressive mimic of a non-suppressive and endogenous disease-driving antigen. Nonlimiting examples of biotherapeutics include protein therapeutics, e.g., antibody therapeutics, fusion protein therapeutics, enzyme therapeutics, viral therapeutics, cell therapeutics, and nucleic acid therapeutics.

Specific examples of biotherapeutics include a monoclonal antibody, a bispecific antibody, an scFv, a Fab, a camelid, or a nanobody, e.g., adalimumab, infliximab, cetuximab, natalizumab, moxetumomab pasudotox, atezolizumab, nivolumab, abciximab, Brentuximab, Certolizumab pegol, elotuzumab, benralizumab, vedolizumab, galcanezumab, rituximab, alemtuzumab, dupilumab, golimumab, obinutuzumab, tildrakizumab, erenumab, mepolizumab, tamucirumab, ranibizumab, ustekinumab, reslizumab, ipilimumab, alirocumab, belimumab, panitumumab, avelumab, necitumumab, mogamulizumab, olaratumab, brodalumab, eculizumab, pertuzumab, pembrolizumab, or tocilizumab. In certain embodiments, the biotherapeutic is erythropoietin, thrombopoietin, human growth hormone, tissue factor, IFNβ-1b, IFNβ-1a, IL-2 or the IL-2 mimetic aldesleukin, exenatide, albiglutide, alefacept, palifermin, or belatacept.

In certain embodiments, the biotherapeutic is an enzyme, such as, asparaginase Erwinia chrysanthemi, phenylalanine ammonia-lyase, alpha-galactosidase A, acid α-glucosidase (GAA), glucocerebrosidase (GCase), aspartylglucosaminidase (AGA), alpha-L-iduronidase, iduronate sulfatase, sulfaminase, α-N-acetylglucosaminidase (NAGLU), heparin acetyle CoA: α-glucosaminide N-acetyltransferase (HGSNAT), N-acetylglucosamine 6-sulfatase (GNS), N-glucosamine 3-O-sulfatase (arylsulfatase G or ARSG), N-acetylgalactosamine 6-sulfatase, beta-galactosidase, N-acetylgalactosamine 4-sulfatase, beta-glucuronidase, Factor VIII, Factor IX, palmitoyl protein thioesterase (PPT1), Tripeptidyl peptidase (TPP1), Pseudomonas elastase (PaE), Pseudomonas alkaline protease (PaAP), or Streptococcal pyrogenic exotoxin B (SpeB). In certain embodiments, the biotherapeutic is not Factor VIII.

In some embodiments, an autoantigen, as provided herein, is a naturally occurring antigen in a healthy individual. Using PANTHER, (Protein ANalysis THrough Evolutionary Relationships), autoantigens can be classified into the following varieties based on function played by the antigen in-vivo that may have a role to play as, for example, an enzyme, intercellular adhesive protein, cell junction protein, cytoskeletal protein, extracellular matrix protein, cellular receptor, transcription or translational protein, gene editing protein, structural protein, or the like.

Autoantigens have been found to be associated with several autoimmune disorders. A comprehensive list of disorders and associated autoantigens can be found at the database AAgAtlas 1.0 database accessed by typing into a web browser http followed by ://biokb.ncpsb.org/followed by aagatlas/). Nonlimiting examples of autoantigens and their associated disorders include: p53-associated autoimmune diseases lupus and scleroderma; Desmoglein (Dsg) 1 and 3 autoantigens associated with Pemphigus vulgaris; autoantigens Gliadin and type 2 transglutaminase associated with Celiac disease; autoantigens PDC-E2 (Pyruvate dehydrogenase complex component E2) and BCOADC-E2 (Branched chain 2-oxo-acid dehydrogenase complex component E2) associated with Primary Biliary Cholangitis; autoantigens PLA2R (Phospholipase A2 Receptor) and THSD7A (thrombospondin type-1 domain containing protein 7A) associated with Membranous Nephropathy; autoantigen TSHR (Thyroid-Stimulating Hormone Receptor) associated with Graves' Disease; autoantigens AChR (Acetylcholine Receptor), MuSK (Muscle-Specific Kinase), and LRP4 (associated with Myasthenia Gravis; and autoantigens associated with rheumatoid arthritis (RA) (e.g., citrullinated peptides and proteins, carbamylated proteins, acetylated proteins) and systemic lupus erythematosus (SLE) (e.g., anti-nuclear antibodies, anti-dsDNA antibodies, anti-nucleosome antibodies, and others), and the like.

In certain aspects, an engineered autoantigen that suppresses an ongoing autoimmune response to the autoantigen in a subject includes a siglec ligand. The engineered autoantigen is configured to both bind to BCRs on a B cell that recognizes the autoantigen and to bind to a siglec present on the B cell. The autoantigen portion of the engineered autoantigen provides specificity for targeting only B cells that bind to the autoantigen while the siglec ligand, while not specific to a particular B cell clone, may bind to any B cell expressing the siglec, prevents activation of the B cell. In some instances, the siglec ligand provides a therapeutic benefit to the individual by suppressing the individual's immune response to the autoantigen, where the immune response is reduced by 50% or more when the engineered autoantigen is administered to an individual relative to when the individual is administered the non-engineered version of the autoantigen. Further, in some instances, the immune reaction is reduced by 60%, 70%, 80% or more, for example 85%, 90%, 95% or more, in certain cases 98%, 99%, or 100%, i.e., such that the immune response is undetectable, i.e., the autoantigen is rendered nonimmunogenic.

In some aspects of the disclosure, an engineered autoantigen is provided, wherein the engineered autoantigen (referred to interchangeably as the “clonally immunosuppressive autoantigen”, “hypoimmunogenic autoantigen”, “modified autoantigen” or simply “subject autoantigen”) is engineered to have an altered Sialic acid-binding immunoglobulin-type lectin (Siglec) ligand (sigL) profile.

Thus, disclosed herein are engineered autoantigens which may retain the epitope(s) recognized by autoimmune antibodies, while comprising one or more modifications comprising addition of a sigL that render the autoantigen capable of suppressing an antigen-specific immune response in an individual to which it has been administered as compared to the unmodified autoantigen. In some embodiments, the immune response is a humoral immune response i.e., a B cell-driven response, e.g., an IgG response.

### Engineered and Hypoimmunogenic

The term “engineered” refers to a biotherapeutic that has been designed and built to comprise one or more modifications relative to biotherapeutic that has not been so engineered, i.e. a parental biotherapeutic, which is also referred to herein as an unengineered biotherapeutic.

By a “hypoimmunogenic” composition, it is meant a composition that suppresses an unwanted, drug-specific immune response in an individual relative to a reference composition, e.g. a corresponding nonengineered composition, when administered to the individual; for example, reducing an immune response by 50% or more relative to a reference, e.g. a nonengineered biotherapeutic, in some instances 60%, 70%, 80% or more, for example 85%, 90%, 95% or more, in certain cases 98%, 99%, or 100%, i.e. such that the immune response is undetectable, i.e. the biotherapeutic is nonimmunogenic. Thus, disclosed herein are engineered biotherapeutics which retain pharmacologic activity while comprising one or more modifications that render the biotherapeutic capable of suppressing a drug-specific immune response in an individual to which it has been administered as compared to the unmodified biotherapeutic. In some embodiments, the immune response is a humoral immune response i.e., a B cell-driven response, e.g. an IgG response.

Conjugates with Multiple Siglec Ligands

As discussed above, the present disclosure describes conjugates of Siglec ligands of formula (I) or (XI) and a biologically active substance. In some cases, these conjugate includes a single Siglec ligand.

In other cases, the conjugates include two or more Siglec ligands and the biologically active substance. In such cases, the conjugates can be indirectly linked to the biologically active substance through a connector (C). Stated in another manner, each of the two or more Siglec ligands can each be covalently bonded to the connector, and the connector can be covalently bonded to the biologically active substance. Likewise, any suitable number of Siglec ligands can be connected to the biologically active substance through the connector (C), such as 3, 4, 5, 6, 7, 8, 9, 10, or more Siglec ligands.

In some cases, a branching location of the connector includes an amino acid residue or a derivative thereof, e.g. lysine or a derivative thereof. Amino acid residues include amino acids commonly found in naturally occurring proteins (e.g., Ala or A, Cys or C, Asp or D, Glu or E, Phe or F, Gly or G, His or H, Ile or I, Lys or K, Leu or L, Met or M, Asn or N, Pro or P, Gln or Q, Arg or R, Ser or S, Thr or T, Val or V, Trp or W, Tyr or Y). In some embodiments, amino acid residues used in the connectors and connector subunits described herein also include amino acid analogs and amino acid derivatives, which are natural amino acids with modified side chains or backbones. Amino acid analogs also include amino acid analogs with the same stereochemistry as in the naturally occurring D-form, as well as the L-form of amino acid analogs. In some instances, the amino acid analogs share backbone structures, and/or the side chain structures of one or more natural amino acids, with difference(s) being one or more modified groups in the molecule. Such modification may include, but is not limited to, substitution of an atom (such as N) for a related atom (such as S), addition of a group (such as methyl, or hydroxyl, etc.) or an atom (such as Cl or Br, etc.), deletion of a group, substitution of a covalent bond (single bond for double bond, etc.), or attachment of another group to the side chain or backbone, or combinations thereof. For example, amino acid analogs may include α-hydroxy acids, and α-amino acids, and the like. In some instances, an amino acid analog or amino acid derivative can include another group, such as another sialic acid moiety (X), attached to the side chain or backbone of the amino acid analog or amino acid derivative through an optional connector.

For instance, the branching location of connector (C) can have the formula shown below, wherein each location marked with an asterisk (*) is a site for heading towards a Siglec group or the biotherapeutic or autoantigen.

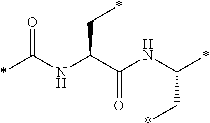 [SMILES: *C[C@@H](*)NC(=O)[C@H](C*)NC(*)=O]

In some cases, the branching location of the connector (C) does not comprise an aryl group or a heteroaryl group. In some cases, the branching location of the connector (C) comprises an alkyl group, an amide group, an amino acid residue group, or a combination thereof.

### Chemical Groups of the Siglec Ligands

Provided by the present disclosure are Siglec ligands with particular chemical groups located at certain positions. The identities of groups are certain locations are applicable to each of formulas (I), (II), (XI), and (XII). In embodiments wherein the identity of X¹, E¹, or E² are specified, such descriptions are also applicable to groups XX¹, EE¹, and EE², where appropriate.

In some cases:
X¹ is O or S;
E¹ is —(CH₂)_{g}—; and
Y is phenylene or substituted phenylene.

In some cases:
X¹ is O or S;
E¹ is —(CH₂)_{g}—;
Y is phenylene or substituted phenylene; and
X² is O.

In some cases:
X¹ is O or S;
E¹ is —(CH₂)_{g}—;
Y is phenylene or substituted phenylene;
X² is O; and
E² is —(CH₂)ₕ—.

In some cases, g is 1, 2, or 3.

In some cases, h is 1, 2, or 3.

In some cases, X¹ is O or S.

In some cases, X¹ is O.

In some cases, X¹ is triazole.

In some cases, E² is —(CH₂)ₕ—.

In some cases, E² is -(alkylene)C(O)NH(alkylene)- or (alkylene)C(O)NH(substituted alkylene)-.

In some cases, Y is phenylene or substituted phenylene.

In some cases, Z is selected from the group consisting of alkyne, azide, thiol, maleimide, iodoacetamide, amine, carboxylic acid or active ester thereof, alkyne, tetrazine, trans-cyclooctene, diene, dienophile, hydroxyl, hydrazido, hydrazino, aldehyde, ketone, azido, phosphine, epoxide, succinimide, and phosphate.

In some cases, Z is selected from the group consisting of alkyne, azide, thiol, maleimide, carboxylic acid or active ester thereof, tetrazine, trans-cyclooctene, diene, dienophile, or phosphate.

In some cases, Z′ is triazole.

In some cases, one or both of A¹ and A² are present.

In some cases, A¹ is —CH₂—.

In some cases, A² is aryl, heteroaryl, or a substituted version thereof.

In some cases, A² is biphenyl, phenylpyridine, phenyl, or a substituted version thereof.

In some cases, R² is —COOH or an ester thereof.

In some cases, R³ is H.

In some cases, R⁴ is hydroxy.

In some cases, R⁵ is —NHC(O)H, —NHC(O)NH₂, or —NHC(O)CH₂OH.

In some cases, R⁵ is —NHC(O)CH₂OH.

In some cases, R⁷ is hydroxy.

### Linkers

As discussed above, in the terminology of formula (I), the “linker” referred to above is the “X¹-E¹-Y—X²-E²” moiety. As such, the “X¹-E¹-Y—X²-E²” group can have advantageous properties that improve the technical qualities of a conjugate. Similarly, in formula (XI) the linker is “XX¹-EE¹-Y—X²-EE²”.

For instance, in some cases the Siglec ligand has the structure shown below:

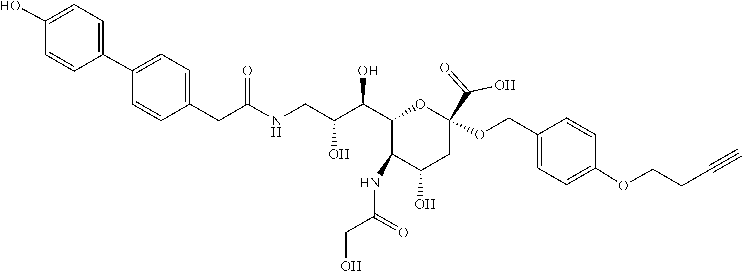 [SMILES: C#CCCOc1ccc(CO[C@]2(C(=O)O)C[C@H](O)[C@@H](NC(=O)CO)[C@H]([C@H](O)[C@H](O)CNC(=O)Cc3ccc(-c4ccc(O)cc4)cc3)O2)cc1]

wherein the linker is

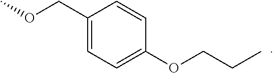

Additional examples of Siglec ligands and their corresponding linkers are shown below.

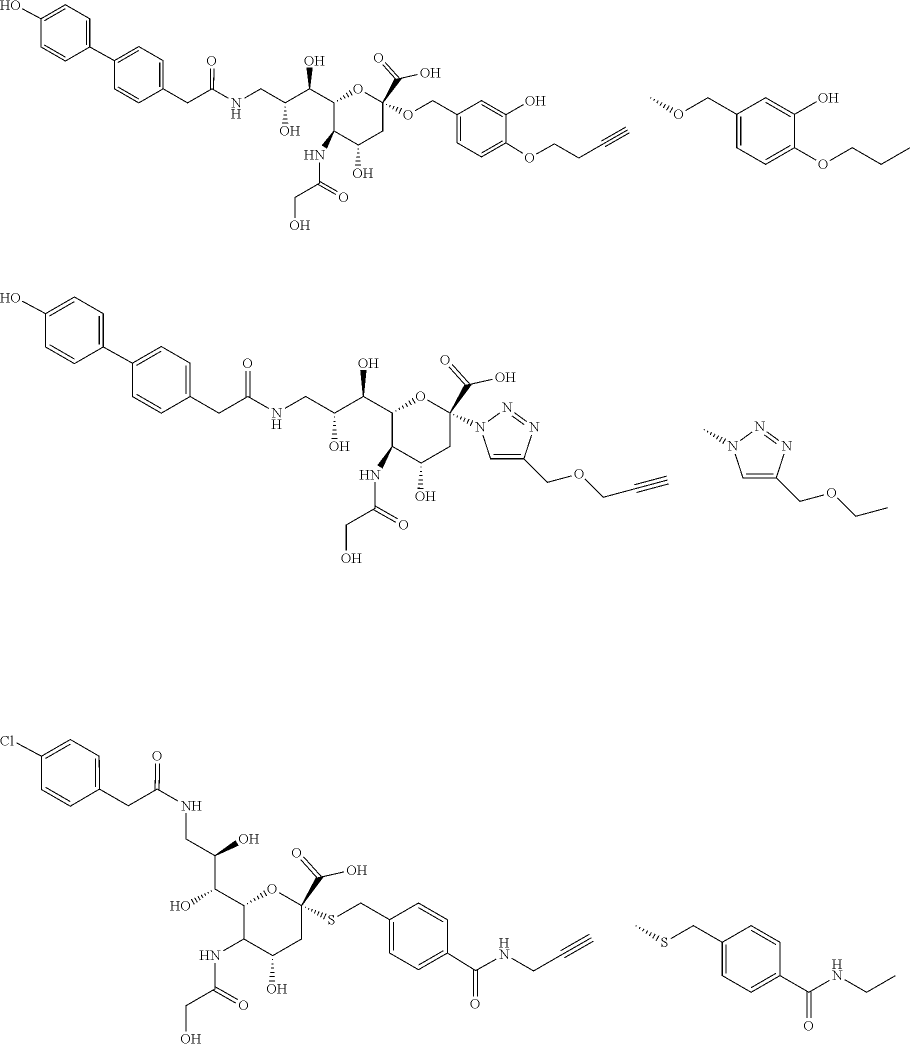

### Pharmaceutical Compositions

Provided are pharmaceutical compositions comprising:
a conjugate as described herein; and
a pharmaceutical excipient.

In methods of treating an individual with the subject hypoimmunogenic biotherapeutic, the patient will typically be administered a pharmaceutical composition comprising the subject hypoimmunogenic biotherapeutic. By a pharmaceutical composition, it is meant an engineered hypoimmunogenic biotherapeutic of the present disclosure that has been formulated in a pharmaceutically acceptable carrier. As used herein, a “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

The pharmaceutical compositions of the disclosure are administered at a therapeutically effective dosage, e.g., a dosage sufficient to provide treatment for the disease states previously described. Administration of the compounds of the disclosure or the pharmaceutically acceptable salts thereof can be via any of the accepted modes of administration for agents that serve similar utilities. While human dosage levels have yet to be optimized for the compounds of the disclosure, these can be readily extrapolated from doses administered to a relevant animal model, e.g. mice that results in treatment of the disease or disorder in that animal model. Generally, an individual human dose is from about 0.01 to 2.0 mg/kg of body weight, preferably about 0.1 to 1.5 mg/kg of body weight, and most preferably about 0.3 to 1.0 mg/kg of body weight. Treatment can be administered for a single day or a period of days, and can be repeated at intervals of several days, one or several weeks, or one or several months. Administration can be as a single dose (e.g., as a bolus) or as an initial bolus followed by continuous infusion of the remaining portion of a complete dose over time, e.g., 1 to 7 days. The amount of active compound administered will, of course, be dependent on any or all of the following: the subject and disease state being treated, the severity of the affliction, the manner and schedule of administration and the judgment of the prescribing physician. It will also be appreciated that amounts administered will depend upon the molecular weight of the biotherapeutic, the amount of Siglec ligand covalently bound, and the size of the linker.

While all typical routes of administration are contemplated (e.g. oral, topical, transdermal, injection (intramuscular, intravenous, or intra-arterial)), it is presently preferred to provide liquid dosage forms suitable for injection. Generally, depending on the intended mode of administration, the pharmaceutically acceptable composition will contain about 0.1% to 95%, preferably about 0.5% to 50%, by weight of the subject hypoimmunogenic biotherapeutic of the disclosure, the remainder being suitable pharmaceutical excipients, carriers, etc. Dosage forms or compositions containing active ingredient in the range of 0.005% to 95% with the balance made up from non-toxic carrier can be prepared.

The subject pharmaceutical compositions can be administered either alone or in combination with other pharmaceutical agents. These compositions can include other medicinal agents, pharmaceutical agents, carriers, and the like, including, but not limited to other active agents that can act as immune-modulating agents and more specifically can have inhibitory effects on B-cells, including anti-folates, immune suppressants, cyostatics, mitotic inhibitors, and anti-metabolites, or combinations thereof.

Liquid pharmaceutically administrable compositions can, for example, be prepared by dissolving, dispersing, etc. an active composition of the disclosure (e.g., a lyophilized powder) and optional pharmaceutical adjuvants in a carrier, such as, for example, water (water for injection), saline, aqueous dextrose, glycerol, glycols, ethanol or the like (excluding galactoses), to thereby form a solution or suspension. If desired, the pharmaceutical composition to be administered can also contain minor amounts of nontoxic auxiliary substances such as wetting agents, emulsifying agents, stabilizing agents, solubilizing agents, pH buffering agents and the like, for example, sodium acetate, sodium citrate, cyclodextrine derivatives, sorbitan monolaurate, triethanolamine acetate and triethanolamine oleate, etc., osmolytes, amino acids, sugars and carbohydrates, proteins and polymers, salts, surfactants, chelators and antioxidants, preservatives, and specific ligands. Actual methods of preparing such dosage forms are known, or will be apparent, to those skilled in this art; for example, see Remington: The Science and Practice of Pharmacy, Pharmaceutical Press, 22nd Edition, 2012. The composition or formulation to be administered will, in any event, contain a quantity of the active compound in an amount effective to treat the symptoms of the subject being treated.

### Methods of Manufacture

Also provided are methods of making the conjugates disclosed herein. Provided is a method of making a conjugate, comprising:
covalently attaching a Siglec ligand as described herein to a biologically active substance,
thereby making the conjugate.

As discussed above, in some cases the conjugate includes two or more Siglec ligands. In some cases, the conjugate includes two or more Siglec ligands that are each covalently bonded to a connector (C), and the connector is covalently bonded to the biologically active substance. In such cases, the method includes covalently bonding each of the two or more Siglec ligands to the connector and covalently bonding the connector to the biologically active substance. The covalently bonding of the Siglec ligands can happen first, or the covalently bonding to the biologically active substance can happen first.

Methods of covalently binding Siglec ligands to biologically active substances (e.g. biotherapeutics) or connectors are well appreciated in the art, any of which may be deployed to modify a biotherapeutic of choice to become an engineered hypoimmunogenic biotherapeutic of the present disclosure. For example, the modification may be performed by engineered biosynthesis. By “biosynthesis”, it is meant a synthesis process that is mediated by cells. For example, in the Golgi apparatus, a subset of the 20 known sialyltransferases attach sialic acids to underlying monosaccharides such as galactose via three different types of linkage (α2,3, α2,6, and α2,8). By engineered biosynthesis, it is meant a synthesis process that is mediated by cells that have been engineered to perform the process, in some instances de novo, in other instances, in a modified way. Thus, for example, a producer cell line may be genetically engineering to express one or more sialyl transferases, e.g. sialyltransferase (EC 2.4.99), beta-galactosamide alpha-2,6-sialyltransferase (EC 2.4.99.1), alpha-N-acetylgalactosaminide alpha-2,6-sialyltransferase (EC 2.4.99.3), beta-galactoside alpha-2,3-sialyltransferase (EC 2.4.99.4), N-acetyllactosaminide alpha-2,3-sialyltransferase (EC 2.4.99.6), alpha-N-acetyl-neuraminide alpha-2,8-sialyltransferase (EC 2.4.99.8); lactosylceramide alpha-2,3-sialyltransferase (EC 2.4.99.9), or other enzymes in an enzymatic pathway, e.g. CMP-Neu5Ac hydroxylase, sialate-4-O-acetyl transferase, sialate-4-O-acetylesterase, sialate-7(9)-O-acetyltransferase, sialate-8-O-methyl transferase, sialate-9-)-acetyltransferase, etc. that drives the covalent binding of a specific sialic acid to the biotherapeutic or that targets specific novel amino acid residues for covalent modification with sialic acid. As another example, a producer cell line could be fed a precursor substrate that will be incorporated by the producer line into the manufactured biotherapeutic as a specific Siglec ligand. Any producer cell that finds use in the expression of proteins for use as therapeutic biotherapeutics may be used in this process, for example a mammalian cell (CHO, HEK, etc.), an insect cell (SF9, etc.), a bacterium, a protozoan (Leishmania, etc.). as disclosed in, e.g. WO2017093291, WO2019002512, WO2019234021, the full disclosures of which are incorporated herein in their entirety by reference.

As another example, the modification may be performed by chemical conjugation. By “chemical conjugation”, it is meant a process that occurs exogenous to a cell. Thus, for example, the Siglec ligand might be enzymatically or chemically linked to the biotherapeutic after biosynthesis from producer cell line. Nonlimiting examples of such in vitro processes are disclosed in U.S. Pat. Nos. 7,220,555, 6,376,475B, and 5,409,817, the full disclosures of which are incorporated herein by reference. In some such embodiments, a connector may be deployed to covalently link the sialic acid to the biotherapeutic. Many examples of connector exist in the art, any of which may be used to chemically conjugate sialic acid(s) to the biotherapeutic to arrive at hypoimmunogenic biotherapeutics of the present disclosure.

As a third example, specifically directed to embodiments in which the Siglec ligand is a peptide or polypeptide sequence, e.g. an scFv or peptide derived from epratuzumab, e.g. PV1, PV2 or PV3, the modification may be performed by genetic engineering of the biotherapeutic to comprise the peptide/polypeptide sequence within the biotherapeutic. For example, the polynucleotide used to produce the biotherapeutic may be modified by standard molecular biology cloning techniques to include a polynucleotide sequence encoding the peptide/polypeptide in the same translational reading frame (“In frame”), such that upon transcription and translation of the biotherapeutic in a producing cell, the biotherapeutic will comprise the peptide/polypeptide sequence covalently associated with amino acids that make up the biotherapeutic, resulting in a biotherapeutic that is hypoimmunogenic. Preferably, the peptide/polypeptide sequence will be genetically engineered into a domain of the biotherapeutic that is not responsible for the therapeutic effect of the biotherapeutic, e.g. the enzymatic domain of an enzyme, the Fab or more specifically CDR domains of an antibody, etc. In the instance of modifying a viral particle, the peptide/polypeptide sequence will preferably be genetically engineered into a capsid or envelop protein so as to be exposed to the exterior of the viral particle, e.g. into an exposed loop of a viral capsid protein, a surface-exposed tegument protein, etc. Such structural features are well understood by one of ordinary skill in the art of viral therapies.

### Methods of Use

Provided are methods of using the conjugates. Provided is a method of treating a patient for a condition, the method comprising:
administering to the patient a conjugate as described herein.

The hypoimmunogenic compositions of the present disclosure find particular use in the treatment of diseases that require repeat or chronic administration of the therapeutic to be effective. There are many instances of such conditions, of which a few nonlimiting examples are provided below and elsewhere. It is expected that the ordinarily skilled artisan will be able to extrapolate from these examples to other indications and biotherapeutics as known in the art.

For example, the individual may be suffering from a chronic autoimmune or inflammatory disease, e.g. rheumatoid arthritis, psoriatic arthritis, ankylosing spondylitis, Crohn's disease, ulcerative colitis, psoriasis, hidradenitis suppurativa, uveitis, and juvenile idiopathic arthritis. In such instances, the method may comprise administering to the individual a hypoimmunogenic TNFα-specific antibody, e.g. a hypoimmunogenic adalimumab engineered from adalimumab, or a hypoimmunogenic infliximab engineered from infliximab, in an amount effective to treat the chronic immune disease.

As another example, the individual may be suffering from a leukemia, e.g. ALL. In such instances, the method may comprise administering to the individual an engineered hypoimmunogenic asparaginase from Erwinia chrysanthemi in an amount effective to treat the leukemia.

As another example, the individual may be suffering from a colorectal cancer, a non-small cell lung cancer, or a head and neck cancer. In such instances, the method may comprise administering to the individual an engineered hypoimmunogenic cetuximab in an amount effective to treat the colorectal cancer, non-small cell lung cancer, or head and neck cancer.

As another example, the individual may be suffering from multiple sclerosis. In such instances, the method may comprise administering to the individual an engineered hypoimmunogenic natalizumab, an engineered hypoimmunogenic IFNβ-1b, or an engineered hypoimmunogenic IFNβ-1a in an amount effective to treat the multiple sclerosis.

As another example, the individual may be the recipient of an organ transplant and in need of an immunosuppressive agent that protects the transplanted tissue from rejection by the individual's immune system. In such instances, the method may comprise administering to the individual an engineered hypoimmunogenic IdeS in an amount effective to prevent an antibody response to the transplanted tissue. In some embodiments, the transplanted organ is an allogeneic graft. In some embodiments, the transplanted organ is a xenogeneic graft. In some embodiments, the organ is selected from kidney, heart, lung, liver, pancreas, trachea, vascular tissue, skin, bone, cartilage, adrenal tissue, fetal thymus, and cornea.

As another example, the individual may be suffering from Type 2 Diabetes. In such instances, the method would comprise administering to the individual an engineered hypoimmunogenic exenatide or engineered hypoimmunogenic albiglutide in an amount effective to treat the diabetes.

As another example, the individual may be suffering from a complement-mediated disease. In such instances, the method would comprise administering to the individual an engineered hypoimmunogenic complement degrading protease, e.g. from a pathogen such as a bacterial pathogen or fungal pathogen (e.g. Pseudomonas Elastase (PaE), Pseudomonas Alkaline protease (PaAP), Streptococcal pyrogenic Exotoxin B (SpeB), a gingipain from Porphyromonas gingivalis, Aspergillus Alkaline protease 1 (Alp1), C. albicans Secreted aspartyl proteinases 1 (Sap1), 2 (Sap2), and 3 (Sap3), in an amount effective to degrade complement and treat the disease.

As another example, the individual may be suffering from an enzyme deficiency. In such instances, the method would comprise administering to the individual an engineered hypoimmunogenic enzyme in an amount effective to treat the deficiency. Nonlimiting examples of such enzyme deficiencies would include PKU, wherein a hypoimmunogenic phenylalanine ammonia-lyase would be administered; Fabry disease, wherein a hypoimmunogenic alpha-galactosidase A would be administered; Pompe disease, wherein a hypoimmunogenic acid α-glucosidase (GAA) would be administered; Gaucher disease, wherein a hypoimmunogenic glucocerebrosidase (GCase) would be administered; Aspartylglucosaminuria, wherein a hypoimmunogenic aspartylglucosaminidase (AGA) would be administered; Hypophosphatasia (HPP), wherein a hypoimmunogenic asfotase would be administered; MPS I, wherein a hypoimmunogenic alpha-L-iduronidase would be administered; MPS II, wherein a hypoimmunogenic iduronate sulfatase would be administered; MPS IIIa, wherein a hypoimmunogenic sulfaminase would be administered; MPS IIIB, wherein a hypoimmunogenic α-N-acetylglucosaminidase (NAGLU) would be administered; MPS IIIC, wherein a hypoimmunogenic heparin acetyle CoA: α-glucosaminide N-acetyltransferase (HGSNAT) would be administered; MPS IIID, wherein a hypoimmunogenic N-acetylglucosamine 6-sulfatase (GNS) would be administered; MPS IIIE, wherein a hypoimmunogenic N-glucosamine 3-O-sulfatase (arylsulfatase G or ARSG) would be administered; MPS IVA, wherein a hypoimmunogenic N-acetylgalactosamine 6-sulfatase would be administered; MPS IVB, wherein a hypoimmunogenic beta-galactosidase would be administered; MPS VI, wherein a hypoimmunogenic N-acetylgalactosamine 4-sulfatase would be administered; MPS VI, wherein a hypoimmunogenic beta-glucuronidase would be administered; Hemophilia A, wherein a hypoimmunogenic Factor VIII would be administered; Hemophilia B, wherein a hypoimmunogenic Factor IX would be administered; the CLN1 form of Batten Disease, wherein a hypoimmunogenic palmitoyl protein thioesterase (PPT1) would be administered; the CLN2 form of Batten Disease, wherein a hypoimmunogenic Tripeptidyl peptidase (TPP1) would be administered; arginase-1 deficiency, wherein a hypoimmunogenic arginase-1 or pegzilarginase would be administered; and cystathionine beta synthase (CBS) deficiency, also known as Classical Homocystinuria, wherein a hypoimmunogenic cystathionine beta synthase or Aeglea product AGLE-177 is administered.

As another example, the individual may be suffering from disease that would benefit from a gene therapy, e.g. a genetic disease, or a complex disease (i.e. not restricted to being associated with a specific genetic etiology) in which chronic expression of a therapeutic RNA or protein would treat the condition. In such instances, the method would comprise administering to the individual an engineered hypoimmunogenic viral particle comprising a polynucleotide sequence (a “transgene”) encoding the therapeutic gene product of interest, in an amount effective to treat the disease. Nonlimiting examples of suitable transgenes/gene products that one might deliver via the subject hypoimmunogenic viral particle include those associated with muscular dystrophy, cystic fibrosis, familial hypercholesterolemia, and rare or orphan diseases. Examples of such rare disease may include spinal muscular atrophy (SMA), Huntingdon's Disease, Rett Syndrome (e.g., methyl-CpG-binding protein 2 (MeCP2); UniProtKB-P51608), Amyotrophic Lateral Sclerosis (ALS), Duchenne Type Muscular dystrophy, Friedrichs Ataxia (e.g., frataxin), ATXN2 associated with spinocerebellar ataxia type 2 (SCA2)/ALS; TDP-43 associated with ALS, progranulin (PRGN) (associated with non-Alzheimer's cerebral degenerations, including, frontotemporal dementia (FTD), progressive non-fluent aphasia (PNFA) and semantic dementia), among others. See, e.g., www.orpha.net/consor/cgi-bin/Disease_Search_List.php; rarediseases.info.nih.gov/diseases.

Other useful therapeutic gene products that could be encoded by the transgene also include hormones and growth and differentiation factors including, without limitation, insulin, glucagon, glucagon-like peptide 1 (GLP-1), growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor a (TGFa), platelet-derived growth factor (PDGF), insulin growth factors I and II (IGF-I and IGF-II), any one of the transforming growth factor b superfamily, including TGF b, activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

Other useful transgenes include those that encode proteins that regulate the immune system including, without limitation, cytokines and lymphokines such as thrombopoietin (TPO), interleukins (IL) IL-1 through IL-25 (including, IL-2, IL-4, IL-12, and IL-18), monocyte chemoattractant protein, leukemia inhibitory factor, granulocyte-macrophage colony stimulating factor, Fas ligand, tumor necrosis factors a and b, interferons a, b, and g, stem cell factor, Hk-2/flt3 ligand. Gene products produced by the immune system are also useful in the invention. These include, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules. Useful gene products also include complement regulatory proteins such as complement regulatory proteins, membrane cofactor protein (MCP), decay accelerating factor (DAF), CR1, CF2 and CD59.

Still other useful transgenes include those that encode gene products for any one of the receptors for the hormones, growth factors, cytokines, lymphokines, regulatory proteins and immune system proteins.

Still other useful transgenes include those encoding receptors for cholesterol regulation, including the low density lipoprotein (LDL) receptor, high density lipoprotein (HDL) receptor, the very low density lipoprotein (VLDL) receptor, and the scavenger receptor. The invention also encompasses gene products such as members of the steroid hormone receptor superfamily including glucocorticoid receptors and estrogen receptors, Vitamin D receptors and other nuclear receptors. In addition, useful gene products include transcription factors such as jun, fos, max, mad, serum response factor (SRF), AP-1, AP2, myb, MyoD and myogenin, ETS-box containing proteins, TFE3, E2F, ATF1, ATF2, ATF3, ATF4, ZF5, NFAT, CREB, HNF-4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

Other useful gene products include, carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-e-phosphatase, porphobilinogen deaminase, Factor VIII, Factor IX, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin sequence or functional fragment thereof. Still other useful gene products include enzymes such as may be useful in enzyme replacement therapy, which is useful in a variety of conditions resulting from deficient activity of enzyme. For example, enzymes that contain mannose-6-phosphate may be utilized in therapies for lysosomal storage diseases (e.g., a suitable gene includes that encodes b-glucuronidase (GUSB)). In another example, the gene product is ubiquitin protein ligase E3A (UBE3A). Still useful gene products include UDP Glucuronosyltransferase Family 1 Member A1 (UGT1A1)

In some embodiment, the gene product is not Factor VIII.

Other useful gene products include non-naturally occurring polypeptides, such as chimeric or hybrid polypeptides having a non-naturally occurring amino acid sequence containing insertions, deletions or amino acid substitutions. For example, single-chain engineered immunoglobulins could be useful in certain immunocompromised patients. Other types of non-naturally occurring gene sequences include antisense molecules and catalytic nucleic acids, such as ribozymes, which could be used to reduce overexpression of a target.

Reduction and/or modulation of expression of a gene is particularly desirable for treatment of hyperproliferative conditions characterized by hyperproliferating cells, as are cancers and psoriasis. Target polypeptides include those polypeptides which are produced exclusively or at higher levels in hyperproliferative cells as compared to normal cells. Target antigens include polypeptides encoded by oncogenes such as myb, myc, fyn, and the translocation gene bcr/abl, ras, src, P53, neu, trk and EGRF. In addition to oncogene products as target antigens, target polypeptides for anti-cancer treatments and protective regimens include variable regions of antibodies made by B cell lymphomas and variable regions of T cell receptors of T cell lymphomas which, in some embodiments, are also used as target antigens for autoimmune disease. Other tumor-associated polypeptides can be used as target polypeptides such as polypeptides which are found at higher levels in tumor cells including the polypeptide recognized by monoclonal antibody 17-1A and folate binding polypeptides.

Other suitable transgenes include those which encode therapeutics that may be useful for treating individuals suffering from autoimmune diseases and disorders by conferring a broad based protective immune response against targets that are associated with autoimmunity including cell receptors and cells which produce self-directed antibodies. T cell mediated autoimmune diseases include Rheumatoid arthritis (RA), multiple sclerosis (MS), Sjogren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative colitis. Each of these diseases is characterized by T cell receptors (TCRs) that bind to endogenous antigens and initiate the inflammatory cascade associated with autoimmune diseases.

Still other useful gene products include those used for treatment of hemophilia, including hemophilia B (including Factor IX) and hemophilia A (including Factor VIII and its variants, such as the light chain and heavy chain of the heterodimer and the B-deleted domain; U.S. Pat. Nos. 6,200,560 and 6,221,349). In some embodiments, the minigene comprises first 57 base pairs of the Factor VIII heavy chain which encodes the 10 amino acid signal sequence, as well as the human growth hormone (hGH) polyadenylation sequence. In alternative embodiments, the minigene further comprises the A1 and A2 domains, as well as 5 amino acids from the N-terminus of the B domain, and/or 85 amino acids of the C-terminus of the B domain, as well as the A3, C1 and C2 domains. In yet other embodiments, the nucleic acids encoding Factor VIII heavy chain and light chain are provided in a single mini gene separated by 42 nucleic acids coding for 14 amino acids of the B domain [U.S. Pat. No. 6,200,560]

Further illustrative genes which may be delivered via the hypoimmunogenic viral particle include, without limitation, glucose-6-phosphatase, associated with glycogen storage disease or deficiency type 1A (GSD1), phosphoenolpyruvate-carboxy kinase (PEPCK), associated with PEPCK deficiency; cyclin-dependent kinase-like 5 (CDKL5), also known as serine/threonine kinase 9 (STK9) associated with seizures and severe neurodevelopmental impairment; galactose-1 phosphate uridyl transferase, associated with galactosemia; phenylalanine hydroxylase (PAH), associated with phenylketonuria (PKU); gene products associated with Primary Hyperoxaluria Type 1 including Hydroxy acid Oxidase 1 (GO/HAO1) and AGXT, branched chain alpha-ketoacid dehydrogenase, including BCKDH, BCKDH-E2, BAKDH-E1a, and BAKDH-E1b, associated with Maple syrup urine disease; fumarylacetoacetate hydrolase, associated with tyrosinemia type 1; methylmalonyl-CoA mutase, associated with methylmalonic acidemia; medium chain acyl CoA dehydrogenase, associated with medium chain acetyl CoA deficiency; ornithine transcarbamylase (OTC), associated with ornithine transcarbamylase deficiency; argininosuccinic acid synthetase (ASS1), associated with citrullinemia; lecithin-cholesterol acyltransferase (LCAT) deficiency; amethylmalonic acidemia (MMA); NPC1 associated with Niemann-Pick disease, type C1); propionic academia (PA); TTR associated with Transthyretin (TTR)-related Hereditary Amyloidosis; low density lipoprotein receptor (LDLR) protein, associated with familial hypercholesterolemia (FH), LDLR variant, such as those described in WO 2015/164778; PCSK9; ApoE and ApoC proteins, associated with dementia; UDP-glucouronosyltransferase, associated with Crigler-Najjar disease; adenosine deaminase, associated with severe combined immunodeficiency disease; hypoxanthine guanine phosphoribosyl transferase, associated with Gout and Lesch-Nyan syndrome; biotimidase, associated with biotimidase deficiency; alpha-galactosidase A (a-Gal A) associated with Fabry disease); beta-galactosidase (GLB1) associated with GM1 gangliosidosis; ATP7B associated with Wilson's Disease; beta-glucocerebrosidase, associated with Gaucher disease type 2 and 3; peroxisome membrane protein 70 kDa, associated with Zellweger syndrome; arylsulfatase A (ARSA) associated with metachromatic leukodystrophy, galactocerebrosidase (GALC) enzyme associated with Krabbe disease, alpha-glucosidase (GAA) associated with Pompe disease; sphingomyelinase (SMPD1) gene associated with Nieman Pick disease type A; argininosuccsinate synthase associated with adult onset type II citrullinemia (CTLN2); carbamoyl-phosphate synthase 1 (CPS1) associated with urea cycle disorders; survival motor neuron (SMN) protein, associated with spinal muscular atrophy; ceramidase associated with Farber lipogranulomatosis; b-hexosaminidase associated with GM2 gangliosidosis and Tay-Sachs and Sandhoff diseases; aspartylglucosaminidase associated with aspartyl-glucosaminuria; a-fucosidase associated with fucosidosis; a-mannosidase associated with alpha-mannosidosis; porphobilinogen deaminase, associated with acute intermittent porphyria (AIP); alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic diseases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson's disease; the beta adrenergic receptor, anti-sense to, or a mutant form of, phospholamban, the sarco(endo)plasmic reticulum adenosine triphosphatase-2 (SERCA2), and the cardiac adenylyl cyclase for the treatment of congestive heart failure; a tumor suppressor gene such as p53 for the treatment of various cancers; a cytokine such as one of the various interleukins for the treatment of inflammatory and immune disorders and cancers; dystrophin or minidystrophin and utrophin or miniutrophin for the treatment of muscular dystrophies; and, insulin or GLP-1 for the treatment of diabetes.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for.

### General Synthetic Procedures

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978)

During any of the processes for preparation of the subject compounds, it may be necessary and/or desirable to protect sensitive or reactive groups on any of the molecules concerned. This may be achieved by means of conventional protecting groups as described in standard works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie”, Houben-Weyl, 4th edition, Vol. 15/1, Georg Thieme Verlag, Stuttgart 1974, in H.-D. Jakubke and H. Jescheit, “Aminosauren, Peptide, Proteine”, Verlag Chemie, Weinheim, Deerfield Beach, and Basel 1982, and/or in Jochen Lehmann, “Chemie der Kohlenhydrate: Monosaccharide and Derivate”, Georg Thieme Verlag, Stuttgart 1974. The protecting groups may be removed at a convenient subsequent stage using methods known from the art.

The subject compounds can be synthesized via a variety of different synthetic routes using commercially available starting materials and/or starting materials prepared by conventional synthetic methods. A variety of examples of synthetic routes that can be used to synthesize the compounds disclosed herein are described in the schemes below.

### Example 1: Synthesis of Compounds

### (2S,4S,5R,6R)-4-hydroxy-5-(2-hydroxyacetamido)-2-((2-(2-(prop-2-yn-1-yloxy)ethoxy)ethyl)thio)-6-((1R,2R)-1,2,3-trihydroxypropyl)tetrahydro-2H-pyran-2-carboxylic acid

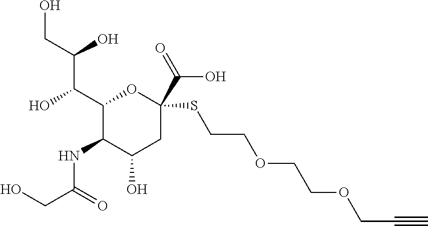 [SMILES: C#CCOCCOCCS[C@]1(C(=O)O)C[C@H](O)[C@@H](NC(=O)CO)[C@H]([C@H](O)[C@H](O)CO)O1]

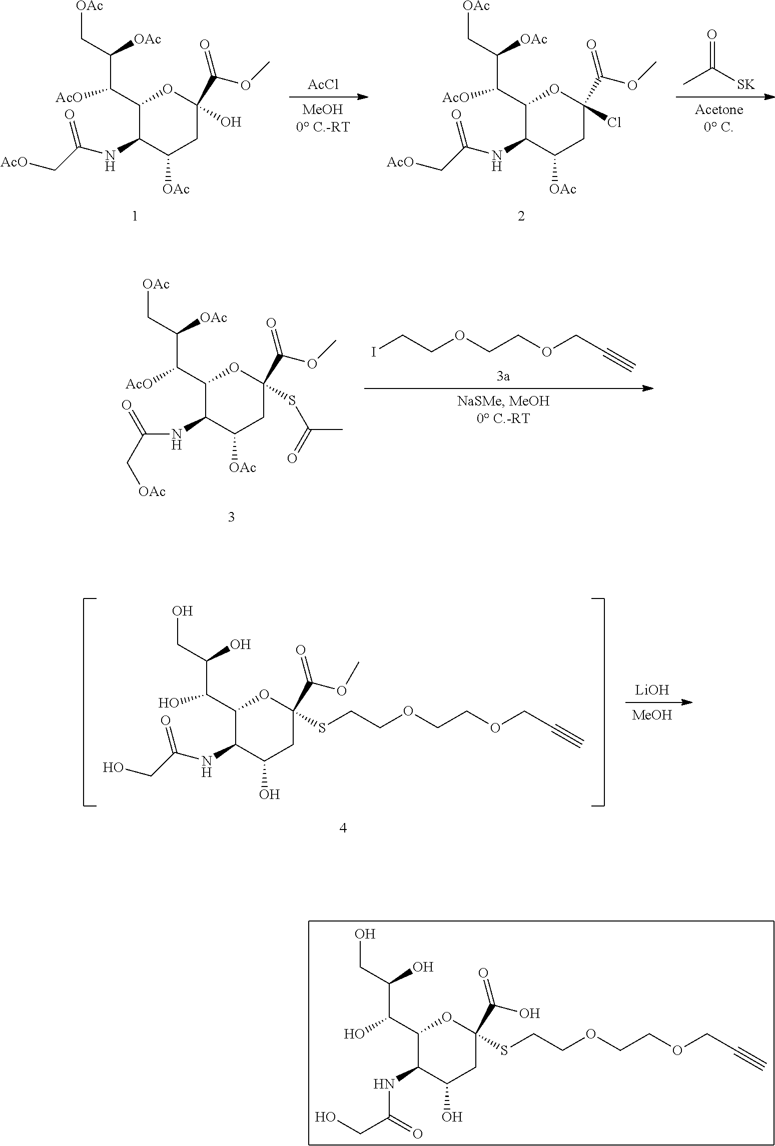

### Synthesis of methyl (2R,4S,5R,6R)-5-amino-4-hydroxy-2-(p-tolylthio)-6-((1R,2R)-1,2,3-trihydroxypropyl)tetrahydro-2H-pyran-2-carboxylate (2)

To a stirred solution (1S,2R)-1-((2R,3R,4S,6R)-4-acetoxy-3-(2-acetoxyacetamido)-6-hydroxy-6-(methoxycarbonyl)tetrahydro-2H-pyran-2-yl)propane-1,2,3-triyl triacetate (1.0 g, 1.82 mmol) in methanol (150.0 mL), was added anhydrous acetic chloride (0.6 mL) dropwise at 0° C. The resulting reaction mixture was stirred at room temperature for 24 h. After completion, the reaction mixture was concentrated to afford the desired product which was used as such for the next step. Yield: 1.0 g, (crude); LC-MS (ESI) m/z 550.48 [M+H]⁺.

### Synthesis of (1S,2R)-1-((2R,3R,4S,6S)-4-acetoxy-3-(2-acetoxyacetamido)-6-(acetylthio)-6-(methoxycarbonyl)tetrahydro-2H-pyran-2-yl)propane-1,2,3-triyl triacetate (3)

The crude product (1S,2R)-1-((2R,3R,4S,6R)-4-acetoxy-3-(2-acetoxyacetamido)-6-chloro-6-(methoxycarbonyl)tetrahydro-2H-pyran-2-yl)propane-1,2,3-triyl triacetate (2, 1.0 g, 1.76 mmol, crude) previously obtained was dissolved under stirring in dry acetone (20.0 mL) and cooled to 0° C. To this solution, potassium thioacetate (0.603 g, 5.28 mmol) was added portionwise at 0° C. and the reaction mixture was stirred for 3 h at 0° C. After completion, the mixture was concentrated and the crude residue obtained was dissolved in ethyl acetate and washed with 1N HCl followed by water. The organic layer was separated, dried over sodium sulfate, and concentrated. The crude residue obtained was purified by column chromatography using ethyl acetate and hexane as eluent to get the desired product. Yield: 0.780 g, 72.91%; LCMS (ESI) m/z 6.8.58 [M+H]⁺.

### Synthesis of (2S,4S,5R,6R)-4-hydroxy-5-(2-hydroxyacetamido)-2-((2-(2-(prop-2-yn-1-yloxy)ethoxy)ethyl)thio)-6-((1R,2R)-1,2,3-trihydroxypropyl)tetrahydro-2H-pyran-2-carboxylic acid

To a stirred solution of (1S,2R)-1-((2R,3R,4S,6S)-4-acetoxy-3-(2-acetoxyacetamido)-6-(acetylthio)-6-(methoxycarbonyl)tetrahydro-2H-pyran-2-yl)propane-1,2,3-triyl triacetate (0.78 g, 1.28 mmol) in methanol (10.0 mL) was added sodium thiomethoxide (0.107 g, 1.54 mmol) at 0° C. After 1 h of stirring, 1-iodo-2-[2-(prop-2-yn-1-yloxy)ethoxy]ethane (0.652 g, 2.57 mmol) was added at 0° C. and the reaction mixture was stirred at room temperature for 1 h. Lithium hydroxide (0.092 g, 3.85 mmol) was added at room temperature and the reaction was stirred 6 h. Dowex-H+ resin was added to reach pH 6 and the mixture was filtered. The filtrate was concentrated, and the crude residue obtained was purified by preparative HPLC using acetonitrile and water (+0.1% TFA) as eluent to afford the desired product. Yield: 0.140 g, 23.33%; ELSD-MS (ESI) m/z 468.2 [M+H]⁺. ¹H NMR (400 MHz, methanol-d₄) δ 7.96 (d, J=7.6 Hz, 1H), 4.18 (d, J=2.4 Hz, 1H), 4.02 (s, 2H), 3.90-3.81 (m, 4H), 3.72-3.57 (m, 8H), 3.52-3.50 (m, 1H), 3.34 (s, 1H), 2.97-2.77 (m, 4H), 1.82-1.76 (t, J=10.8 Hz, 1H).

### (2R,4S,5R,6R)-2-((4-(but-3-yn-1-yloxy)benzyl)oxy)-6-((1R,2R)-1,2-dihydroxy-3-(2-(4′-hydroxy-[1,1′-biphenyl]-4-yl)acetamido)propyl)-4-hydroxy-5-(2-hydroxyacetamido)tetrahydro-2H-pyran-2-carboxylic acid (Compound 77)

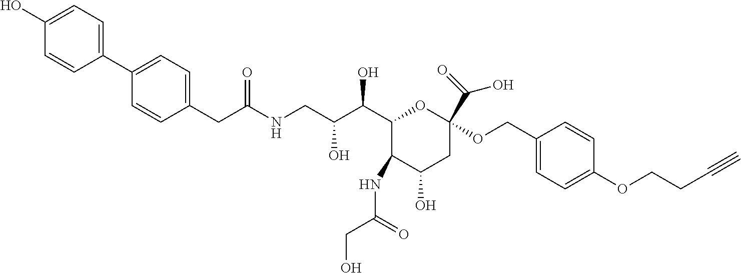 [SMILES: C#CCCOc1ccc(CO[C@]2(C(=O)O)C[C@H](O)[C@@H](NC(=O)CO)[C@H]([C@H](O)[C@H](O)CNC(=O)Cc3ccc(-c4ccc(O)cc4)cc3)O2)cc1]

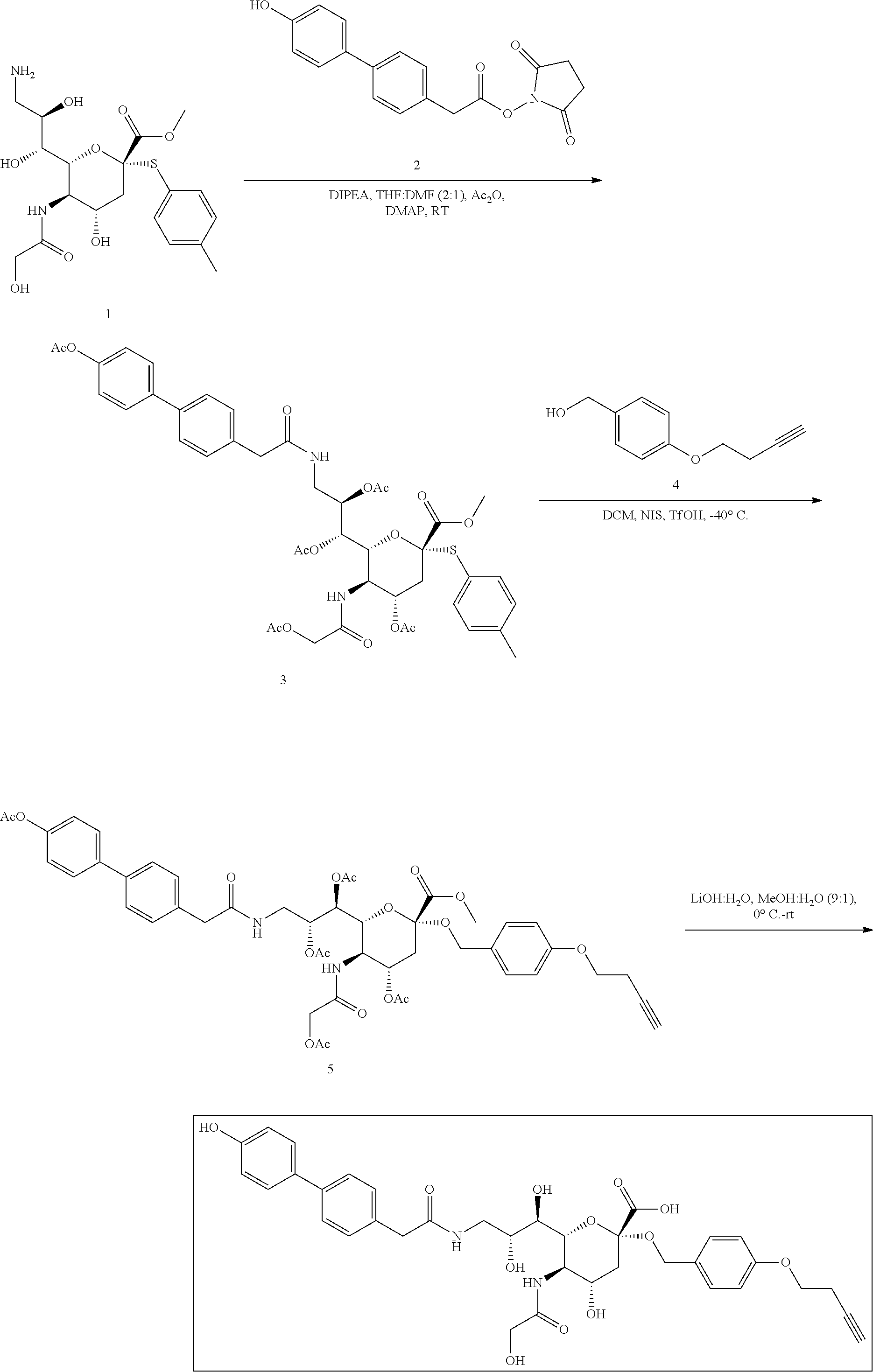

### Synthesis of (1R,2R)-1-((2R,3R,4S,6S)-4-acetoxy-3-(2-acetoxyacetamido)-6-(methoxycarbonyl)-6-(p-tolylthio)tetrahydro-2H-pyran-2-yl)-3-(2-(4′-acetoxy-[1,1′-biphenyl]-4-yl)acetamido)propane-1,2-diyl diacetate (3)

To a stirred solution of methyl (2S,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (1.5 g, 3.37 mmol) and 2,5-dioxopyrrolidin-1-yl 2-(4′-hydroxy-[1,1′-biphenyl]-4-yl)acetate (0.878 g, 2.700 mmol) in tetrahydrofuran (10.00 mL) and N,N-Dimethylformamide (5.0 mL) was added DIPEA (0.938 mL, 5.38 mmol) at 0° C. and the reaction mixture was stirred at room temperature for 4 h. Acetic anhydride (3.19 mL, 33.70 mmol) and 4-dimethylaminopyridine (0.205 g, 1.680 mmol) were added at 0° C. and the mixture was stirred at room temperature for 12 h. The mixture was diluted with water and ethyl acetate. The organic layer was separated, dried, filtered and concentrated. The crude residue obtained was purified by column chromatography using ethyl acetate and hexane as eluent to afford the desired product. Yield: 1.10 g, 38%; LCMS (ESI) m/z 865.01 [M+H]⁺.

### Synthesis of (1R,2R)-1-((2R,3R,4S,6R)-4-acetoxy-3-(2-acetoxyacetamido)-6-((4-(but-3-yn-1-yloxy)benzyl)oxy)-6-(methoxycarbonyl)tetrahydro-2H-pyran-2-yl)-3-(2-(4′-acetoxy-[1,1′-biphenyl]-4-yl)acetamido)propane-1,2-diyl diacetate (5)

A mixture of (1R,2R)-1-((2R,3R,4S,6S)-4-acetoxy-3-(2-acetoxyacetamido)-6-(methoxycarbonyl)-6-(p-tolylthio)tetrahydro-2H-pyran-2-yl)-3-(2-(4′-acetoxy-[1,1′-biphenyl]-4-yl)acetamido)propane-1,2-diyl diacetate (3, 1.10 g, 1.270 mmol) and 2-[2-(prop-2-yn-1-yloxy)ethoxy]ethan-1-ol (4, 0.672 g, 3.820 mmol) and activated 4 Å powdered molecular sieves (1.00 g) in anhydrous dichloromethane (14.0 ml) was stirred at room temperature for 15 h under nitrogen. 1-Iodopyrrolidine-2,5-dione (0.715 g, 3.180 mmol) and a solution of trifluoromethanesulfonic acid (0.112 mL, 1.270 mmol) in dichloromethane (1.0 mL) were added at −40° C. After 1 h at this temperature, the reaction mixture was quenched with addition of triethyl amine (1.0 mL) and warmed to room temperature. The reaction mixture was filtered, the filtrate was washed with a saturated solution of sodium bicarbonate, dried over sodium sulfate, filtered and concentrated. The crude product was purified by column chromatography using ethyl acetate and hexane as eluent to afford the desired product. Yield: 0.700 g, 60%; LCMS m/z 914.65 [M−1]⁻.

### Synthesis of (2R,4S,5R,6R)-2-((4-(but-3-yn-1-yloxy)benzyl)oxy)-6-((1R,2R)-1,2-dihydroxy-3-(2-(4′-hydroxy-[1,1′-biphenyl]-4-yl)acetamido)propyl)-4-hydroxy-5-(2-hydroxyacetamido)tetrahydro-2H-pyran-2-carboxylic acid (Compound 77)

To a stirred solution of (1R,2R)-1-((2R,3R,4S,6R)-4-acetoxy-3-(2-acetoxyacetamido)-6-((4-(but-3-yn-1-yloxy)benzyl)oxy)-6-(methoxycarbonyl)tetrahydro-2H-pyran-2-yl)-3-(2-(4′-acetoxy-[1,1′-biphenyl]-4-yl)acetamido)propane-1,2-diyl diacetate (5, 0.700 g, 0.350 mmol) in methanol (7.0 mL), was added a solution of lithium hydroxide monohydrate (0.199 g, 4.74 mmol) in water (1.0 mL). The reaction mixture was stirred at room temperature for 6 h then treated with Dowex 50 resin to reach pH ˜6. The suspension was filtered, and the filtrate was concentrated. The residue obtained was purified by prep HPLC using acetonitrile and water (+0.1% TFA) as eluent to afford the desired product. Yield: 0.149 g, 28%; LCMS m/z 693.45 [M+H]⁺; ¹H NMR (400 MHz, methanol-d₄) δ 7.94 (t, J=5.2 Hz, 1H), 7.89 (d, J=7.6 Hz, 1H), 7.45 (d, J=8.0 Hz, 2H), 7.38 (dt, J=9.6 & 2.0 Hz, 2H), 7.31 (d, J=4.0 Hz, 2H), 7.22 (d, J=8.8 Hz, 2H), 4.72 (d, J=11.20 Hz, 1H), 4.40 (d, J=10.80 Hz, 1H), 4.03-4.02 (m, 4H), 3.96-3.91 (m, 1H), 3.86-3.81 (m, 2H), 3.79-3.76 (m, 1H), 3.73-3.69 (m, 1H), 3.59-3.53 (m, 2H), 3.40 (dd, J=8.4 & 1.2 Hz, 1H), 3.28-3.25 (m, 1H), 2.77 (dd, J=12.8 & 4.0 Hz, 1H), 2.62 (td, J=6.4 & 2.4 Hz, 2H), 2.31 (t, J=2.8 Hz, 1H), 1.75 (t, J=12.4 Hz, 1H).

### (2R,4S,5R,6R)-2-((4-(but-3-yn-1-yloxy)-3-hydroxybenzyl)oxy)-6-((1R,2R)-1,2-dihydroxy-3-(2-(4′-hydroxy-[1,1′-biphenyl]-4-yl)acetamido)propyl)-4-hydroxy-5-(2-hydroxyacetamido)tetrahydro-2H-pyran-2-carboxylic acid (Compound 73)

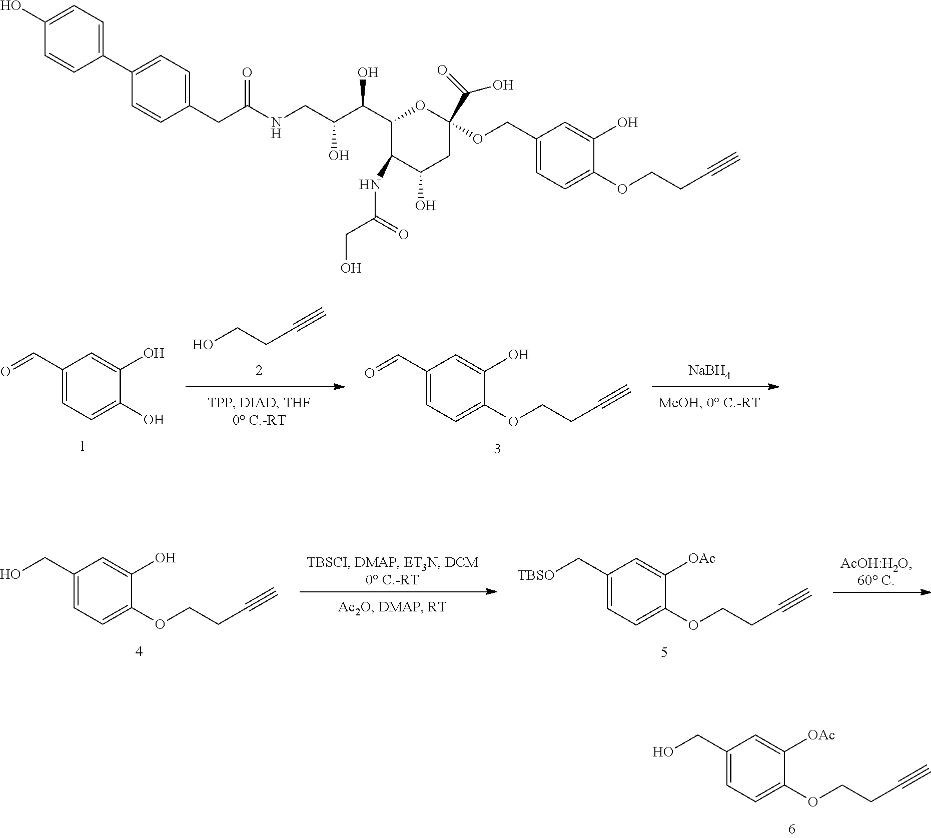

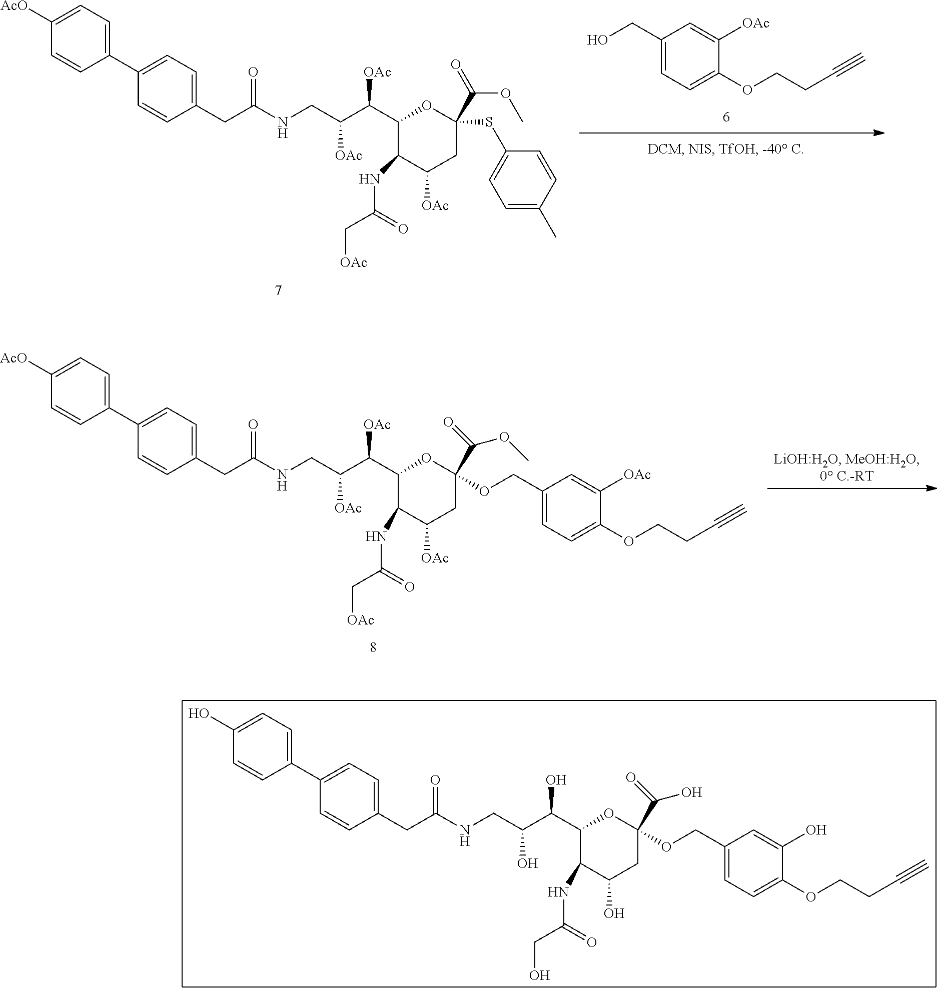 [SMILES: COC(=O)[C@@]1(Sc2ccc(C)cc2)C[C@H](OC(C)=O)[C@@H](NC(=O)COC(C)=O)[C@H]([C@H](OC(C)=O)[C@@H](CNC(=O)Cc2ccc(-c3ccc(OC(C)=O)cc3)cc2)OC(C)=O)O1 ; C#CCCOc1ccc(CO)cc1OC(C)=O ; C#CCCOc1ccc(CO[C@]2(C(=O)OC)C[C@H](OC(C)=O)[C@@H](NC(=O)COC(C)=O)[C@H]([C@H](OC(C)=O)[C@@H](CNC(=O)Cc3ccc(-c4ccc(OC(C)=O)cc4)cc3)OC(C)=O)O2)cc1OC(C)=O ; "LiOH:H2O, MeOH:H2O,0° C.-RT" ; C#CCCOc1ccc(CO[C@]2(C(=O)O)C[C@H](O)[C@@H](NC(=O)CO)[C@H]([C@H](O)[C@H](O)CNC(=O)Cc3ccc(-c4ccc(O)cc4)cc3)O2)cc1O]

### Synthesis of 4-(but-3-yn-1-yloxy)-3-hydroxybenzaldehyde (3)

To a solution of 3,4-dihydroxybenzaldehyde (5.0 g, 36.20 mmol) in tetrahydrofuran (50 ml), but-3-yn-1-ol (2, 2.54 g, 36.20 mmol), triphenylphosphine (16.10 g, 61.50 mmol) and diisopropyl azodicarboxylate (DIAD) (11.0 g, 54.30 mmol) were added at 0° C., the reaction mixture was stirred at room temperature for 24 h. After completion, solvent was concentrated under high vacuum and the crude was purified by column chromatography using ethyl acetate and hexane as eluent to afford 4-(but-3-yn-1-yloxy)-3-hydroxybenzaldehyde (3) as off white solid. Yield: 2.5 g, 36%; LCMS (ESI) m/z 191.15 [M+H]⁺.

### Synthesis of 2-(but-3-yn-1-yloxy)-5-(hydroxymethyl)phenol (4)

To a solution of 4-(but-3-yn-1-yloxy)-3-hydroxybenzaldehyde (2.50 g, 13.10 mmol), in methanol (30 mL), was added sodium borohydride (1.24 g, 32.90 mmol) at 0° C. and the reaction mixture was stirred at room temperature for 2 h. After completion, the reaction was quenched with ice cold water and the solvent was concentrated. The crude residue obtained was diluted with 1N HCl to pH=4, then extracted with dichloromethane. The organic layer was dried over sodium sulfate, filtered, and concentrated. The crude residue obtained was purified by silica-gel column chromatography using ethyl acetate and hexane as eluents to afford the desired product. Yield: 2.2 g, 87%; LC-MS (ESI) m/z 191.01 [M−1]⁻.

### Synthesis of 2-(but-3-yn-1-yloxy)-5-(((tert-butyldimethylsilyl)oxy)methyl)phenyl acetate (5)

To a solution of 2-(but-3-yn-1-yloxy)-5-(hydroxymethyl)phenol (4, 2.20 g, 11.40 mmol), in dichloromethane (30 mL), trimethylamine (3.32 mL, 22.90 mmol), 4-dimethylaminopyridine (1.54 g, 12.60 mmol) and tert-butyl(chloro)dimethylsilane (TBSCl) (1.73 g, 11.40 mmol) were successively added at 0° C., and the reaction mixture was stirred at room temperature for 6 h. After completion, acetic anhydride (2.16 mL, 22.80 mmol) and 4-dimethylaminopyridine (1.40 g, 11.4 mmol) were added at 0° C. and the reaction mixture was stirred at room temperature for 12 h. The reaction mixture was diluted with water and extracted with dichloromethane. The organic layer was dried, filtered, and concentrated. The crude residue obtained was purified by silica-gel column chromatography using ethyl acetate and hexane as eluents to afford the desired product. Yield: 2.50 g, 64%; LC-MS (ESI) m/z 347.05 [M−1]⁻.

### Synthesis of 2-(but-3-yn-1-yloxy)-5-(hydroxymethyl)phenyl acetate (6)

A solution of 2-(but-3-yn-1-yloxy)-5-(((tert-butyldimethylsilyl)oxy)methyl)phenyl acetate (5, 2.5 g, 7.17 mmol) in acetic acid:water (9:1) (20 ml) was stirred at 60° C. for 6 h. The solvent was concentrated and the crude residue obtained was purified by silica-gel column chromatography using ethyl acetate and hexane as eluents to afford the desired product. Yield: 1.50 g, 89%; LC-MS (ESI) m/z 233.05 [M−1]⁻.

### Synthesis of (1R,2R)-1-((2R,3R,4S,6R)-4-acetoxy-6-((3-acetoxy-4-(but-3-yn-1-yloxy)benzyl)oxy)-3-(2-acetoxyacetamido)-6-(methoxycarbonyl)tetrahydro-2H-pyran-2-yl)-3-(2-(4′-acetoxy-[1,1′-biphenyl]-4-yl)acetamido)propane-1,2-diyl diacetate (8)

A mixture of (1R,2R)-1-((2R,3R,4S,6S)-4-acetoxy-3-(2-acetoxyacetamido)-6-(methoxycarbonyl)-6-(p-tolylthio)tetrahydro-2H-pyran-2-yl)-3-(2-(4′-acetoxy-[1,1′-biphenyl]-4-yl)acetamido)propane-1,2-diyl diacetate (1.20 g, 1.38 mmol), 2-(but-3-yn-1-yloxy)-5-(hydroxymethyl)phenyl acetate (0.976 g, 4.16 mmol) and activated powdered 4 Å molecular sieves (1.00 g, 100% w/w) in anhydrous dichloromethane (25.0 mL) was stirred at room temperature for 15 h. The solution was then cooled to −40° C. followed by addition of 1-iodopyrrolidine-2,5-dione (0.840 g, 3.46 mmol) and trifluoromethanesulfonic acid (0.20 mL, 1.38 mmol). The reaction mixture was stirred at this temperature for another 2 h. The reaction mixture was quenched with addition of triethyl amine (neutral pH) and warmed gradually to room temperature. The reaction mixture was filtered, and the filtrate was washed with water. The organic layer was separated, dried, filtered and concentrated. The crude residue obtained was purified by column chromatography using ethyl acetate and hexane as eluent to afford the desired product. Yield: 0.930 g, 69%; LCMS m/z 974.90 [M+H]⁺.

### Synthesis of (2R,4S,5R,6R)-2-((4-(but-3-yn-1-yloxy)-3-hydroxybenzyl)oxy)-6-((1R,2R)-1,2-dihydroxy-3-(2-(4′-hydroxy-[1,1′-biphenyl]-4-yl)acetamido)propyl)-4-hydroxy-5-(2-hydroxyacetamido)tetrahydro-2H-pyran-2-carboxylic acid

To a stirred solution of (1R,2R)-1-((2R,3R,4S,6R)-4-acetoxy-6-((3-acetoxy-4-(but-3-yn-1-yloxy)benzyl)oxy)-3-(2-acetoxyacetamido)-6-(methoxycarbonyl)tetrahydro-2H-pyran-2-yl)-3-(2-(4′-acetoxy-[1,1′-biphenyl]-4-yl)acetamido)propane-1,2-diyl diacetate (8, 0.930 g, 0.95 mmol) in methanol (10.0 mL), was added a solution of lithium hydroxide monohydrate (0.137 g, 5.72 mmol) in water (1.0 mL). The reaction mixture was stirred at room temperature for 4 h then treated with Dowex 50 resin to reach pH 6 and the suspension was filtered. The filtrate was concentrated, and the crude residue obtained was purified by prep HPLC using acetonitrile and water (+0.1% TFA) as eluent to afford the desired product. Yield: 0.105 g, 15.5%; LCMS m/z 709.40 [M+H]⁺; ¹H NMR (400 MHz, methanol-d₄) (7.47 (d, J=8.4 Hz, 2H), 7.42-7.37 (m, 2H), 7.32 (d, J=8.0 Hz, 2H), 6.83-6.81 (m, 4H), 6.72-6.70 (m, 1H), 4.68 (d, J=10.8 Hz, 1H), 4.37 (d, J=10.8 Hz, 1H), 4.10 (t, J=6.8 Hz, 2H), 4.02 (brs, 2H), 3.95-3.56 (m, 7H), 3.42-3.96 (m, 1H), 3.28-3.25 (m, 1H), 2.76 (dd, J=4 Hz, 12.4 Hz, 1H), 2.68-2.64 (m, 2H), 2.34 (t, J=2.4 Hz, 1H), 1.80 (t, J=12.4 Hz, 1H).

### (2S,4S,5R,6R)-2-((4-(but-3-yn-1-yloxy)benzyl)thio)-6-((1R,2R)-1,2-dihydroxy-3-(2-(4′-hydroxy-[1,1′-biphenyl]-4-yl)acetamido)propyl)-4-hydroxy-5-(2-hydroxyacetamido)tetrahydro-2H-pyran-2-carboxylic acid (Compound 64)

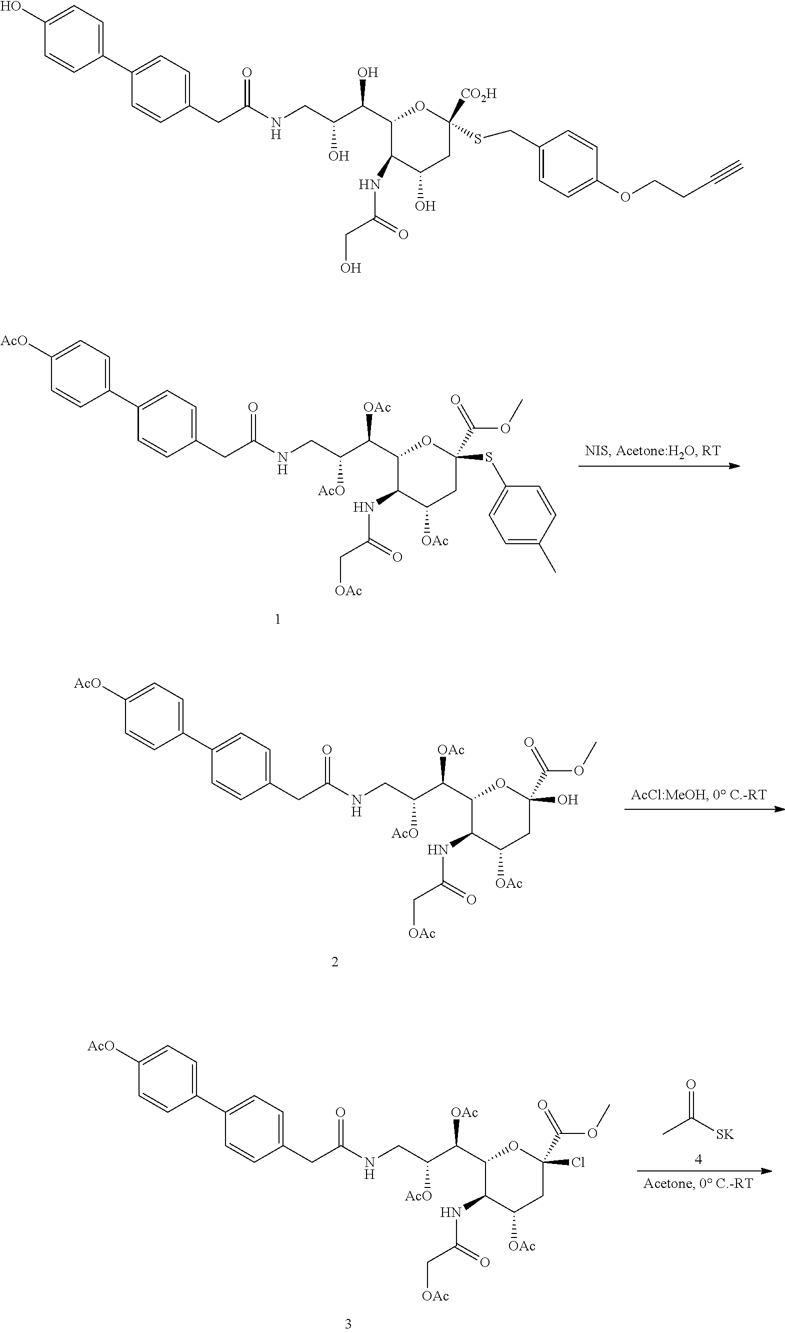

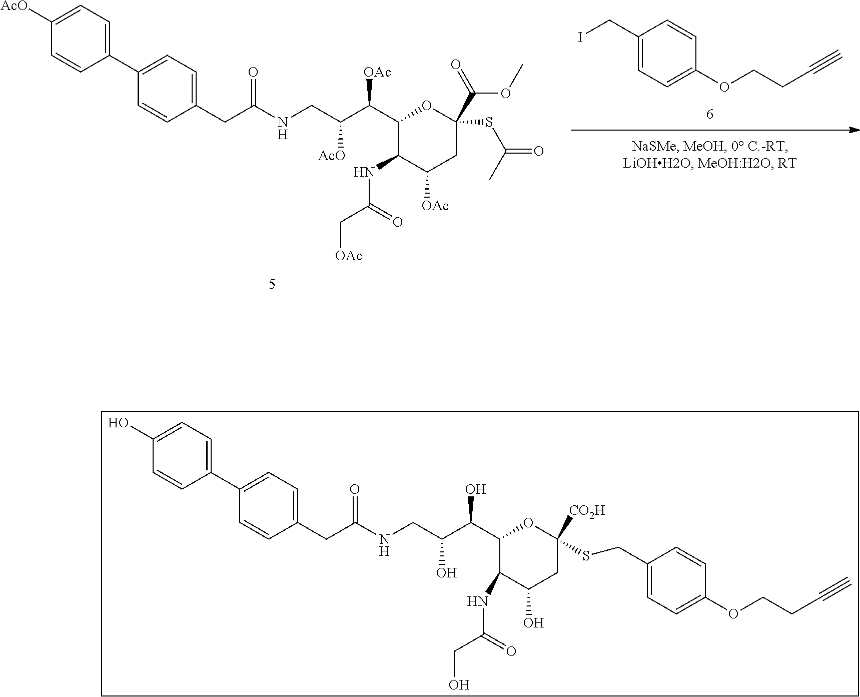 [SMILES: COC(=O)[C@@]1(SC(C)=O)C[C@H](OC(C)=O)[C@@H](NC(=O)COC(C)=O)[C@H]([C@H](OC(C)=O)[C@@H](CNC(=O)Cc2ccc(-c3ccc(OC(C)=O)cc3)cc2)OC(C)=O)O1 ; C#CCCOc1ccc(CI)cc1 ; C#CCCOc1ccc(CS[C@]2(C(=O)O)C[C@H](O)[C@@H](NC(=O)CO)[C@H]([C@H](O)[C@H](O)CNC(=O)Cc3ccc(-c4ccc(O)cc4)cc3)O2)cc1]

### Synthesis of (1R,2R)-1-((2R,3R,4S,6S)-4-acetoxy-3-(2-acetoxyacetamido)-6-hydroxy-6-(methoxycarbonyl)tetrahydro-2H-pyran-2-yl)-3-(2-(4′-acetoxy-[1,1′-biphenyl]-4-yl)acetamido)propane-1,2-diyl diacetate (2)

A stirred solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4′-acetoxy-[1,1′-biphenyl]-4-yl)acetamido)propane-1,2-diyl diacetate (0.70 g, 0.81 mmol) in acetone:water (9:1, 10.0 mL) was cooled to 0° C. To this solution, N-iodosuccinimide (0.686 g, 2.83 mmol) was added and the reaction was maintained at 0° C. until completion. Saturated aqueous solution of sodium metabisulfide (10.0 mL) and ethyl acetate (20.0 mL) was added, and the reaction mixture was stirred for another 10 min. The product was extracted with ethyl acetate. The organic layers were combined and washed successively with saturated sodium bicarbonate solution and water, dried, filtered, and concentrated. The crude residue obtained was purified by column chromatography using ethyl acetate and hexane as eluent to afford the desired product. Yield: 0.50 g, 81%; LCMS (ESI) m/z 759.15 [M+H]⁺.

### Synthesis of (1R,2R)-1-((2R,3R,4S,6R)-4-acetoxy-3-(2-acetoxyacetamido)-6-chloro-6-(methoxycarbonyl)tetrahydro-2H-pyran-2-yl)-3-(2-(4′-acetoxy-[1,1′-biphenyl]-4-yl)acetamido)propane-1,2-diyl diacetate (3)

To a stirred solution of (1R,2R)-1-((2R,3R,4S,6S)-4-acetoxy-3-(2-acetoxyacetamido)-6-hydroxy-6-(methoxycarbonyl)tetrahydro-2H-pyran-2-yl)-3-(2-(4′-acetoxy-[1,1′-biphenyl]-4-yl)acetamido)propane-1,2-diyl diacetate (2, 0.50 g, 0.66 mmol) in acetyl chloride (30.0 mL), was added anhydrous methanol (1.0 mL) dropwise at 0° C. The resulting reaction mixture was stirred at room temperature for 24 h then concentrated. The crude residue obtained was used as such for the next step. Yield: 0.40 g, (crude).

### Synthesis of (1R,2R)-1-((2R,3R,4S,6S)-4-acetoxy-3-(2-acetoxyacetamido)-6-(acetylthio)-6-(methoxycarbonyl)tetrahydro-2H-pyran-2-yl)-3-(2-(4′-acetoxy-[1,1′-biphenyl]-4-yl)acetamido)propane-1,2-diyl diacetate (5)

(1R,2R)-1-((2R,3R,4S,6R)-4-acetoxy-3-(2-acetoxyacetamido)-6-chloro-6-(methoxycarbonyl)tetrahydro-2H-pyran-2-yl)-3-(2-(4′-acetoxy-[1,1′-biphenyl]-4-yl)acetamido)propane-1,2-diyl diacetate (3, 0.40 g, 0.52 mmol, crude) was dissolved under stirring in dry acetone (10.0 mL) and cooled to 0° C. Potassium thioacetate (4, 0.176 g, 1.54 mmol) was added portion wise at 0° C.; the reaction was stirred for 3 h at 0° C. then concentrated. The crude residue obtained was taken up in ethyl acetate and washed successively with 1N HCl and water. The organic layer was dried, filtered, then concentrated. The crude residue obtained was purified by column chromatography using ethyl acetate and hexane as eluent to afford the desired product. Yield: 0.35 g, 83%; LCMS (ESI) m/z 817.15 [M+H]⁺.

### Synthesis of (2S,4S,5R,6R)-2-((4-(but-3-yn-1-yloxy)benzyl)thio)-6-((1R,2R)-1,2-dihydroxy-3-(2-(4′-hydroxy-[1,1′-biphenyl]-4-yl)acetamido)propyl)-4-hydroxy-5-(2-hydroxyacetamido)tetrahydro-2H-pyran-2-carboxylic acid (Compound 64)

To a stirred solution of (1R,2R)-1-((2R,3R,4S,6S)-4-acetoxy-3-(2-acetoxyacetamido)-6-(acetylthio)-6-(methoxycarbonyl)tetrahydro-2H-pyran-2-yl)-3-(2-(4′-acetoxy-[1,1′-biphenyl]-4-yl)acetamido)propane-1,2-diyl diacetate (5, 0.350 g, 0.49 mmol) in methanol (5.0 mL) was added sodium thiomethoxide (0.050 g, 0.588 mmol) at 0° C. After 1 h of stirring, 1-(But-3-yn-1-yloxy)-4-(iodomethyl)benzene (6, 0.280 g, 0.98 mmol) was added at 0° C. and the reaction mixture was stirred at room temperature for 1 h. Lithium hydroxide (0.041 g, 0.98 mmol) was added and the reaction was stirred at room temperature for 6 h. Dowex-hydrogen resin was added to reach pH 6 and the resin was filtered off. The filtrate was concentrated, and the crude residue obtained was purified by prep HPLC using acetonitrile and water (+0.1% TFA) as eluent to afford the desired product. Yield: 0.0045 g, 1.5%; LCMS (ESI) m/z 709.35 [M+H]⁺. ¹H NMR (400 MHz, methanol-d₄) δ 7.41 (d, J=8.0 Hz, 2H), 7.35 (d, J=8.4 Hz, 2H), 7.29 (d, J=8.4 Hz, 2H), 7.18 (d, J=8.8 Hz, 2H), 6.80 (dd, J=8.4 & 6.4 Hz, 4H), 4.01-3.98 (m, 5H), 3.95-3.85 (m, 3H), 3.83-3.77 (m, 1H), 3.70 (dd, J=13.6 & 2.8 Hz, 1H), 3.64 (d, J=8.8 Hz, 1H), 3.55 (d, J=3.6 Hz, 2H), 3.37 (d, J=8.8 Hz, 1H), 3.23-3.17 (m, 1H), 2.80-2.76 (m, 1H), 2.61 (dt, J=6.8 & 2.8 Hz, 2H), 2.30 (t, J=2.4 Hz, 1H), 1.77 (d, J=11.2 Hz, 1H).

### (2R,4S,5R,6R)-6-((1R,2R)-1,2-dihydroxy-3-(2-(4′-hydroxy-[1,1′-biphenyl]-4-yl)acetamido)propyl)-4-hydroxy-5-(2-hydroxyacetamido)-2-(4-((prop-2-yn-1-yloxy)methyl)-1H-1,2,3-triazol-1-yl)tetrahydro-2H-pyran-2-carboxylic acid (Compound 62)

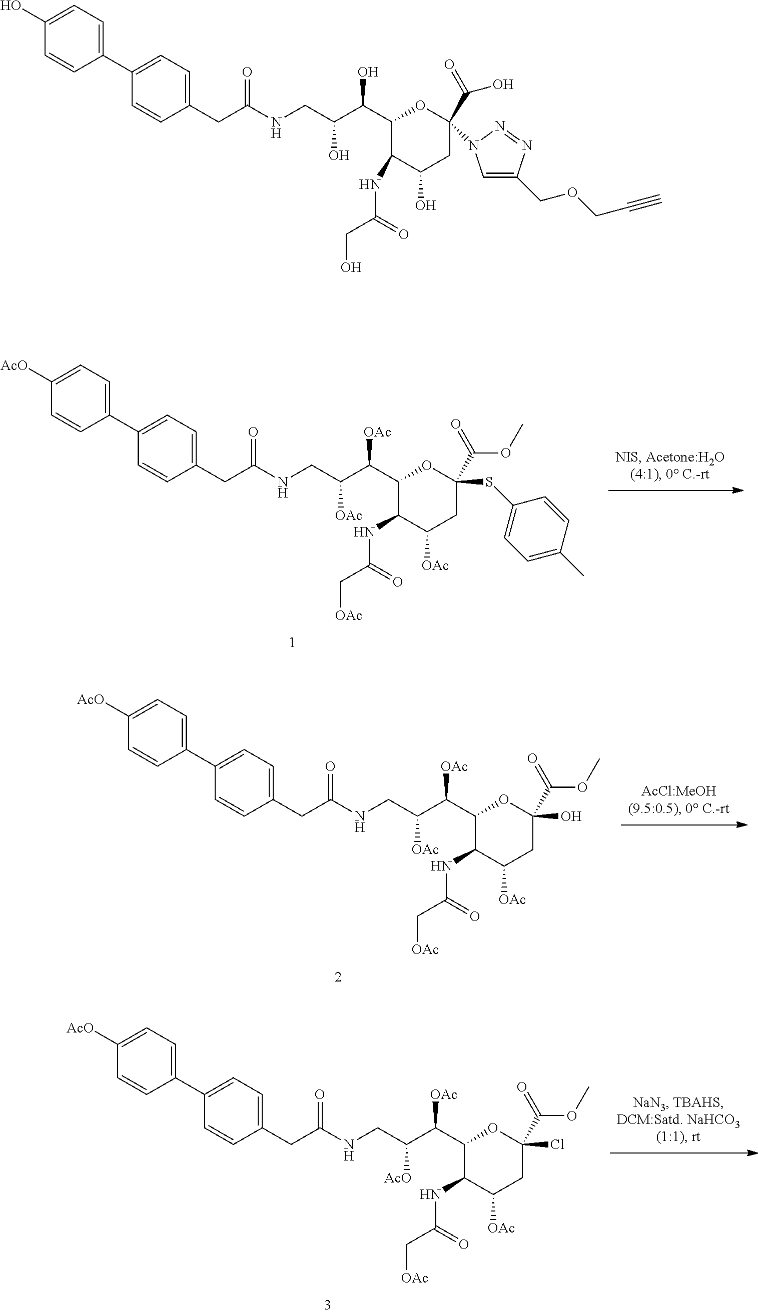

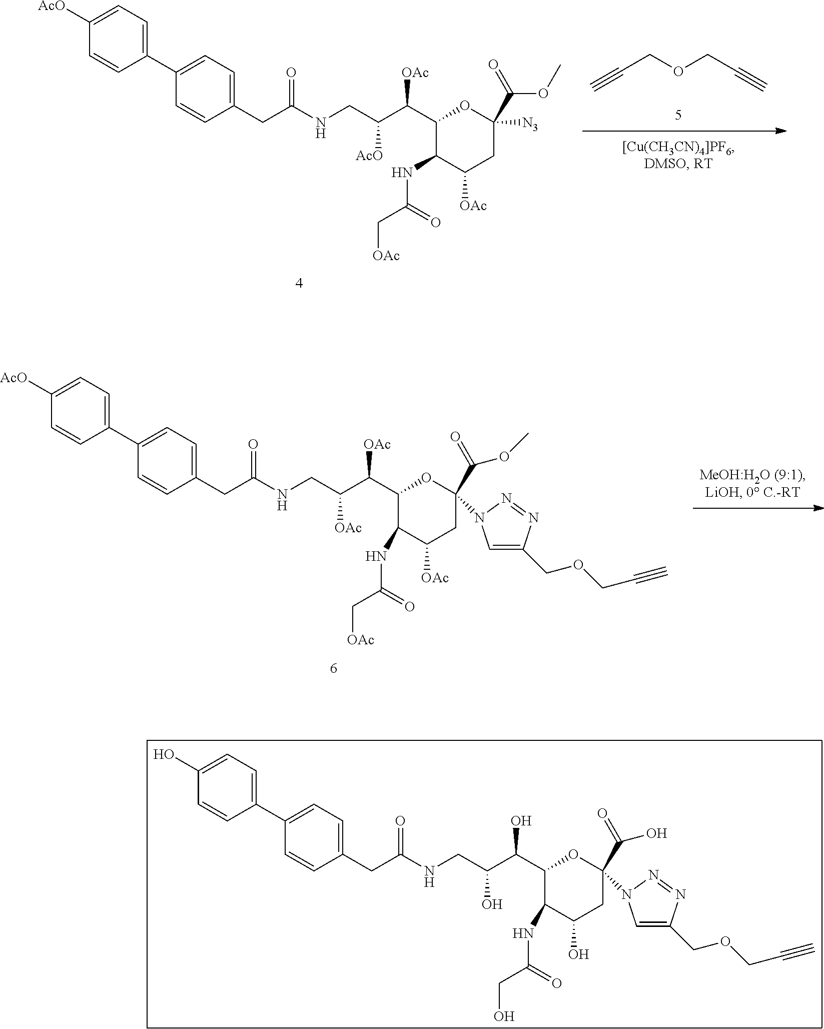

### Synthesis of (1R,2R)-1-((2R,3R,4S,6S)-4-acetoxy-3-(2-acetoxyacetamido)-6-hydroxy-6-(methoxycarbonyl)tetrahydro-2H-pyran-2-yl)-3-(2-(4′-acetoxy-[1,1′-biphenyl]-4-yl)acetamido)propane-1,2-diyl diacetate (2)

A stirred solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4′-acetoxy-[1,1′-biphenyl]-4-yl)acetamido)propane-1,2-diyl diacetate (0.70 g, 0.81 mmol) in acetone:water (9:1, 10.0 mL) was cooled to 0° C. To this solution, N-iodosuccinimide (0.686 g, 2.83 mmol) was added and the reaction was maintained at 0° C. for 3 h. After completion, saturated aqueous solution of sodium metabisulfite (10.0 mL) and ethyl acetate (20.0 mL) was added to the reaction mixture. The reaction mixture was stirred for 10 min. The organic layer was separated, and the aqueous phase was washed with ethyl acetate (10 mL). The organic layers were combined and washed sequentially with saturated sodium bicarbonate solution and water. The organic layer was dried and concentrated. The crude residue obtained was purified by column chromatography using ethyl acetate and hexane as eluent to afford the desired product. Yield: 0.50 g, 81%; LCMS (ESI) m/z 759.15 [M+H]⁺.

### Synthesis of (1R,2R)-1-((2R,3R,4S,6R)-4-acetoxy-3-(2-acetoxyacetamido)-6-chloro-6-(methoxycarbonyl)tetrahydro-2H-pyran-2-yl)-3-(2-(4′-acetoxy-[1,1′-biphenyl]-4-yl)acetamido)propane-1,2-diyl diacetate (3)

To a stirred solution of (1R,2R)-1-((2R,3R,4S,6S)-4-acetoxy-3-(2-acetoxyacetamido)-6-hydroxy-6-(methoxycarbonyl)tetrahydro-2H-pyran-2-yl)-3-(2-(4′-acetoxy-[1,1′-biphenyl]-4-yl)acetamido)propane-1,2-diyl diacetate (2, 0.50 g, 0.66 mmol) in acetyl chloride (30.0 mL), was added anhydrous methanol (1.0 mL) dropwise at 0° C. The resulting reaction mixture was stirred at room temperature for 24 h. After completion, the reaction mixture was concentrated to afford the desired crude product. Yield: 0.40 g (crude).

### Synthesis of (1R,2R)-1-((2R,3R,4S,6R)-4-acetoxy-3-(2-acetoxyacetamido)-6-azido-6-(methoxycarbonyl)tetrahydro-2H-pyran-2-yl)-3-(2-(4′-acetoxy-[1,1′-biphenyl]-4-yl)acetamido)propane-1,2-diyl diacetate (4)

To a stirred solution of (1R,2R)-1-((2R,3R,4S,6R)-4-acetoxy-3-(2-acetoxyacetamido)-6-chloro-6-(methoxycarbonyl)tetrahydro-2H-pyran-2-yl)-3-(2-(4′-acetoxy-[1,1′-biphenyl]-4-yl)acetamido)propane-1,2-diyl diacetate (3, 1.0 g, 1.29 mmol) in dichloromethane (20 ml) was added a solution of tetrabutylammonium hydrogensulfate (0.437 g, 1.29 mmol) and sodium azide (0.418 g, 6.43 mmol) in saturated aqueous solution of sodium bicarbonate (20 mL). The mixture was stirred for 0.5 h. The organic layer was washed successively with 2 M HCl (20 mL), saturated aqueous sodium bicarbonate solution (20 mL), and brine (30 mL). The organic layer was filtered, and the filtrate was concentrated. The crude residue obtained was purified by column chromatography using ethyl acetate and hexane as eluent to afford the desired product. Yield: 0.9 g, 89%; LCMS (ESI) m/z 784.10 [M+H]⁺.

### Synthesis of (1R,2R)-1-((2R,3R,4S,6R)-4-acetoxy-3-(2-acetoxyacetamido)-6-(methoxycarbonyl)-6-(4-((prop-2-yn-1-yloxy)methyl)-1H-1,2,3-triazol-1-yl)tetrahydro-2H-pyran-2-yl)-3-(2-(4′-acetoxy-[1,1′-biphenyl]-4-yl)acetamido)propane-1,2-diyl diacetate (6)

To a stirred solution of (1R,2R)-1-((2R,3R,4S,6R)-4-acetoxy-3-(2-acetoxyacetamido)-6-azido-6-(methoxycarbonyl)tetrahydro-2H-pyran-2-yl)-3-(2-(4′-acetoxy-[1,1′-biphenyl]-4-yl)acetamido)propane-1,2-diyl diacetate (4, 0.20 g, 0.25 mmol) in DMSO (5 mL), 3-(prop-2-yn-1-yloxy)prop-1-yne (5, 0.05 g, 0.50 mmol) and λ¹-copper(1+) tetrakis(acetonitrile) hexafluoride λ⁻⁵-phosphanepentauide (0.285 g, 0.75 mmol) were added sequentially. The reaction mixture was stirred for 12 h at room temperature. The solution was concentrated, and the crude was purified by column chromatography using ethyl acetate and hexane as eluent to afford the desired product. Yield: 0.2 g, 89%; LCMS m/z 878.1 [M+H]+

### Synthesis of (2R,4S,5R,6R)-6-((1R,2R)-1,2-dihydroxy-3-(2-(4′-hydroxy-[1,1′-biphenyl]-4-yl)acetamido)propyl)-4-hydroxy-5-(2-hydroxyacetamido)-2-(4-((prop-2-yn-1-yloxy)methyl)-1H-1,2,3-triazol-1-yl)tetrahydro-2H-pyran-2-carboxylic acid (Compound 62)

To a stirred solution of (1R,2R)-1-((2R,3R,4S,6R)-4-acetoxy-3-(2-acetoxyacetamido)-6-(methoxycarbonyl)-6-(4-((prop-2-yn-1-yloxy)methyl)-1H-1,2,3-triazol-1-yl)tetrahydro-2H-pyran-2-yl)-3-(2-(4′-acetoxy-[1,1′-biphenyl]-4-yl)acetamido)propane-1,2-diyl diacetate (6, 0.20 g, 0.23 mmol) in methanol (5.0 mL), was added a solution of lithium hydroxide monohydrate (0.033 g, 1.37 mmol) in water (0.5 mL). The reaction mixture was stirred at room temperature for 6 h. The reaction mixture was treated with acidic resin (Dowex 50H⁺) to pH ˜6 and the suspension was filtered. The filtrate was concentrated, and the crude residue obtained was purified by prep HPLC using acetonitrile and water (+0.1% TFA) as eluent to afford the desired product. Yield: 0.047 g, 32%; LCMS m/z 654.40[M+H]⁺; ¹H NMR (400 MHz, methanol-d₄): δ 8.29 (s, 1H), 8.00 (d, J=8.4 Hz, 1H), 7.50 (d, J=8.4 Hz, 2H), 7.45-7.43 (m, 2H), 7.32 (d, J=8.0 Hz, 2H), 6.85-6.83 (m, 2H), 4.67 (s, 2H), 4.19 (d, J=2.4 Hz, 2H), 4.17-3.87 (m, 6 h), 3.65 (dd, J=13.6 & 3.2 Hz, 1H), 3.56 (s, 2H), 3.48-3.42 (m, 1H), 3.35-3.31 (m, 1H), 3.27-3.24 (m, 1H), 2.87 (t, J=2.4 Hz, 1H), 2.22 (t, J=12 Hz, 1H).

### (2R,4S,5R,6R)-6-((1R,2R)-1,2-dihydroxy-3-(2-(4′-hydroxy-[1,1′-biphenyl]-4-yl)acetamido)propyl)-4-hydroxy-5-(2-hydroxyacetamido)-2-(4-(4-(prop-2-yn-1-yloxy)phenyl)-1H-1,2,3-triazol-1-yl)tetrahydro-2H-pyran-2-carboxylic acid (Compound 53)

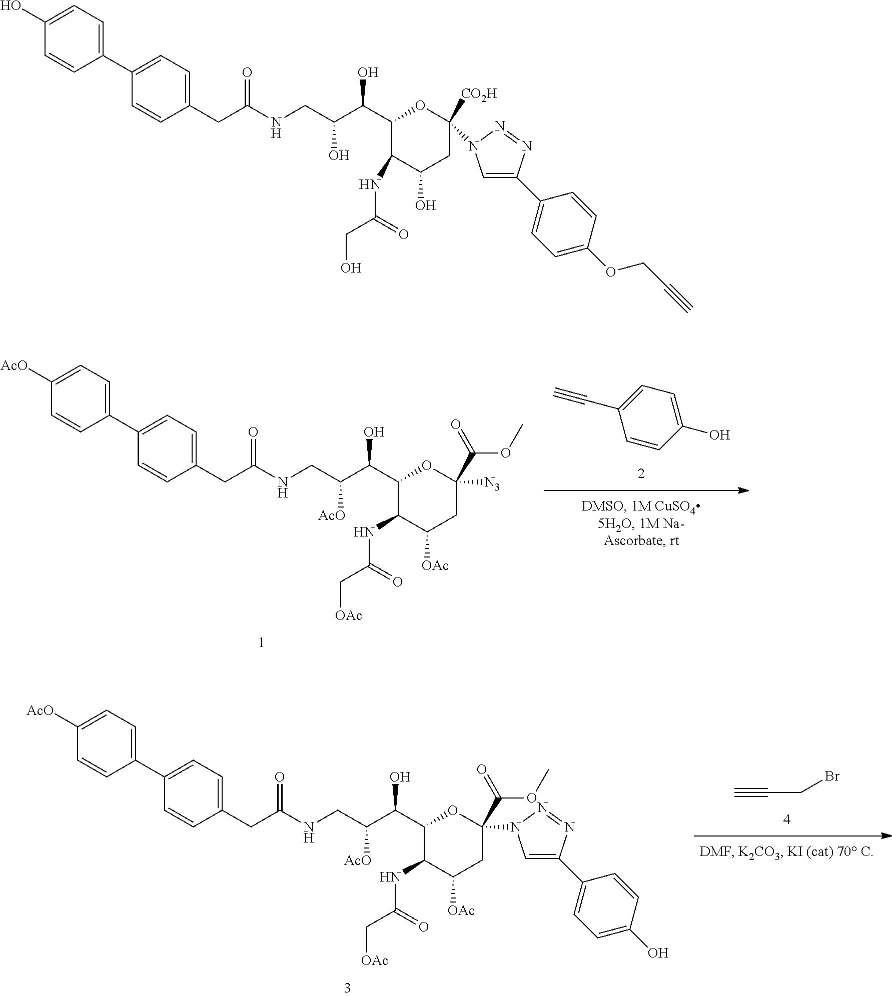

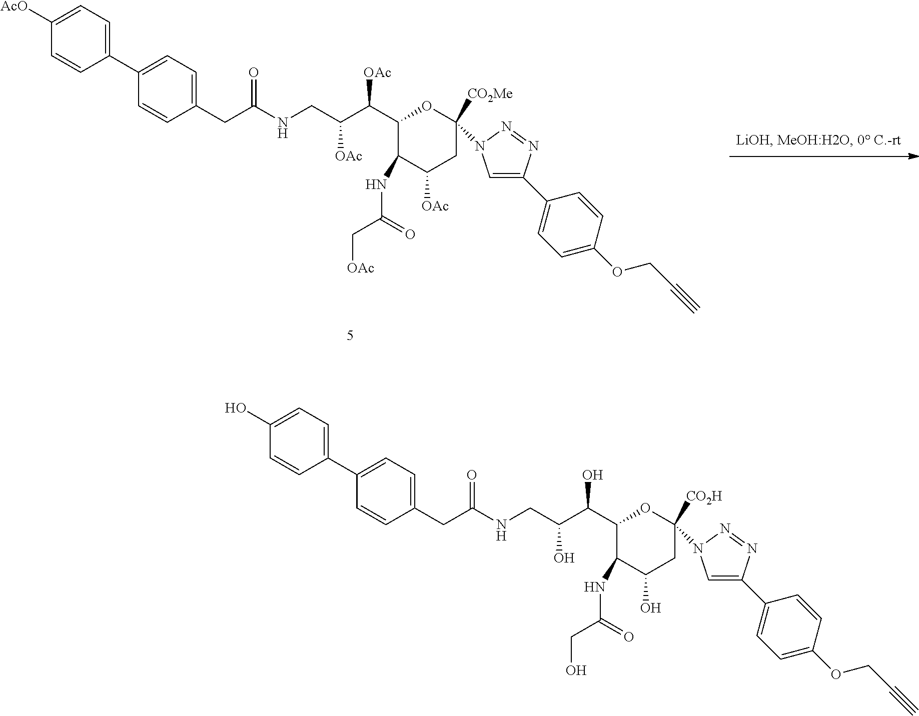 [SMILES: C#CCOc1ccc(-c2cn([C@]3(C(=O)OC)C[C@H](OC(C)=O)[C@@H](NC(=O)COC(C)=O)[C@H]([C@H](OC(C)=O)[C@@H](CNC(=O)Cc4ccc(-c5ccc(OC(C)=O)cc5)cc4)OC(C)=O)O3)nn2)cc1 ; C#CCOc1ccc(-c2cn([C@]3(C(=O)O)C[C@H](O)[C@@H](NC(=O)CO)[C@H]([C@H](O)[C@H](O)CNC(=O)Cc4ccc(-c5ccc(O)cc5)cc4)O3)nn2)cc1]

### Synthesis of (1R,2R)-1-((2R,3R,4S,6R)-4-acetoxy-3-(2-acetoxyacetamido)-6-(4-(4-hydroxyphenyl)-1H-1,2,3-triazol-1-yl)-6-(methoxycarbonyl)tetrahydro-2H-pyran-2-yl)-3-(2-(4′-acetoxy-[1,1′-biphenyl]-4-yl)acetamido)propane-1,2-diyl diacetate (3)

To a stirred solution of (1R,2R)-1-((2R,3R,4S,6R)-4-acetoxy-3-(2-acetoxyacetamido)-6-azido-6-(methoxycarbonyl)tetrahydro-2H-pyran-2-yl)-3-(2-(4′-acetoxy-[1,1′-biphenyl]-4-yl)acetamido)propane-1,2-diyl diacetate (0.20 g, 0.25 mmol) in dimethyl sulfoxide (5 mL) were added 4-ethynylphenol (2, 0.09 g, 0.76 mmol), 1 M CuSO4 solution (5 ml) and 1 M sodium 5-[(1S)-1,2-dihydroxyethyl]-3-hydroxy-2,4-dioxooxolan-3-ide (5 ml) sequentially. The reaction mixture was stiired for 12 h at room temperature. The organic layer was washed with brine (30 mL). The combined extracts were concentrated. The crude residue obtained was purified by silica gel column chromatography using ethyl acetate and heptane as eluent to afford the desired product. Yield: 0.11 g; 47.8%; LCMS m/z 902.9 [M+H]+;

### Synthesis of (1R,2R)-1-((2R,3R,4S,6R)-4-acetoxy-3-(2-acetoxyacetamido)-6-(methoxycarbonyl)-6-(4-(4-(prop-2-yn-1-yloxy)phenyl)-1H-1,2,3-triazol-1-yl)tetrahydro-2H-pyran-2-yl)-3-(2-(4′-acetoxy-[1,1′-biphenyl]-4-yl)acetamido)propane-1,2-diyl diacetate (5)

To a solution of (1R,2R)-1-((2R,3R,4S,6R)-4-acetoxy-3-(2-acetoxyacetamido)-6-(4-(4-hydroxyphenyl)-1H-1,2,3-triazol-1-yl)-6-(methoxycarbonyl)tetrahydro-2H-pyran-2-yl)-3-(2-(4′-acetoxy-[1,1′-biphenyl]-4-yl)acetamido)propane-1,2-diyl diacetate (3, 0.25 g, 0.27 mmol) in N,N-dimethylformamide (5.0 mL), were added dipotassium carbonate (0.076 g, 0.554 mmol) and potassium iodide (0.004 g, 0.027 mmol) and the reaction mixture was heated at 50° C. for 1 h. Then 3-bromoprop-1-yne (66.0 mg, 0.55 mmol) was added dropwise to the reaction mixture which was stirred at room temperature for another 12 h. The reaction mixture was diluted with ethyl acetate and washed with water. The organic layer was dried, filtered and the filtrate was concentrated. The crude residue obtained was purified by silica gel column chromatography using ethyl acetate and heptane as eluent to afford the desired product. Yield: 0.20 g, 76%.

### Synthesis of (2R,4S,5R,6R)-6-((1R,2R)-1,2-dihydroxy-3-(2-(4′-hydroxy-[1,1′-biphenyl]-4-yl)acetamido)propyl)-4-hydroxy-5-(2-hydroxyacetamido)-2-(4-(4-(prop-2-yn-1-yloxy)phenyl)-1H-1,2,3-triazol-1-yl)tetrahydro-2H-pyran-2-carboxylic acid (Compound 53)

To a stirred solution of (1R,2R)-1-((2R,3R,4S,6R)-4-acetoxy-3-(2-acetoxyacetamido)-6-(methoxycarbonyl)-6-(4-(4-(prop-2-yn-1-yloxy)phenyl)-1H-1,2,3-triazol-1-yl)tetrahydro-2H-pyran-2-yl)-3-(2-(4′-acetoxy-[1,1′-biphenyl]-4-yl)acetamido)propane-1,2-diyl diacetate (5, 0.15 g, 0.16 mmol) in methanol (5.0 mL), was added a solution of lithium hydroxide monohydrate (0.023 g, 0.95 mmol) in water (0.5 mL). The reaction mixture was stirred at room temperature for 6 h. The reaction mixture was treated with acidic resin (Dowex 50H⁺) to reach pH ˜6, the suspension was filtered. The filtrate was concentrated, and the crude residue obtained was purified by prep HPLC using acetonitrile and water (+0.1% TFA) as eluent to afford the desired product. Yield: 0.062 g, 42%; LCMS m/z 716.3 [M+H]⁺; ¹H NMR (400 MHz, methanol-d₄) δ ¹H NMR (400 MHz, Methanol-d₄) δ 8.58 (s, 1H), 7.76 (d, J=8.8 Hz, 2H), 7.48 (d, J=8.0 Hz, 2H), 7.41 (d, J=8.8 Hz, 2H), 7.31 (d, J=8.0 Hz, 2H), 7.04 (d, J=8.8 Hz, 2H), 6.84 (d, J=8.0 Hz, 2H), 4.74 (d, J=2.4 Hz, 2H), 4.20-3.92 (m, 6 h), 3.69 (dd, J=14.0 & 2.8 Hz, 1H), 3.56 (s, 2H), 3.47 (d, J=8.4 Hz, 1H), 3.38-3.35 (m, 1H), 3.27-3.25 (m, 1H), 2.96 (t, J=2.4 Hz, 1H), 2.26 (t, J=12.0 Hz, 1H).

### (2R,4S,5R,6R)-6-((1R,2R)-1,2-dihydroxy-3-(2-(4′-hydroxy-[1,1′-biphenyl]-4-yl)acetamido)propyl)-4-hydroxy-5-(2-hydroxyacetamido)-2-(4-(3-(prop-2-yn-1-yloxy)phenyl)-1H-1,2,3-triazol-1-yl)tetrahydro-2H-pyran-2-carboxylic acid (Compound 60)

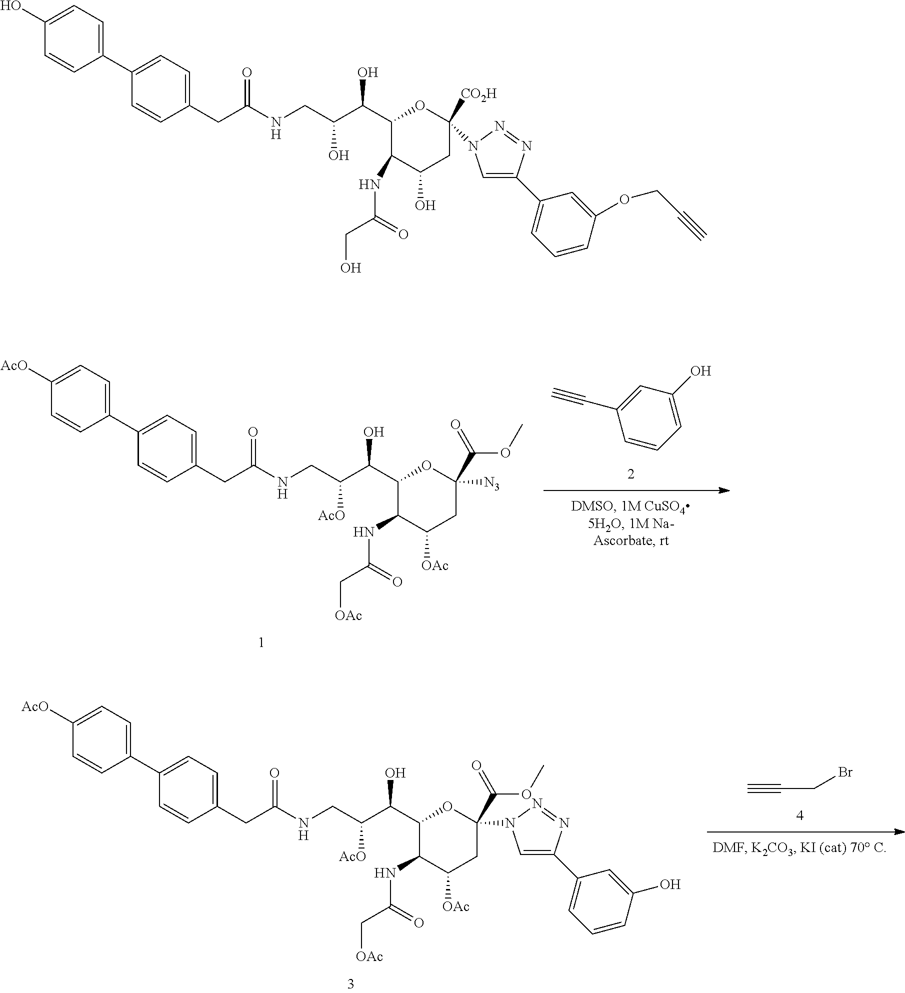

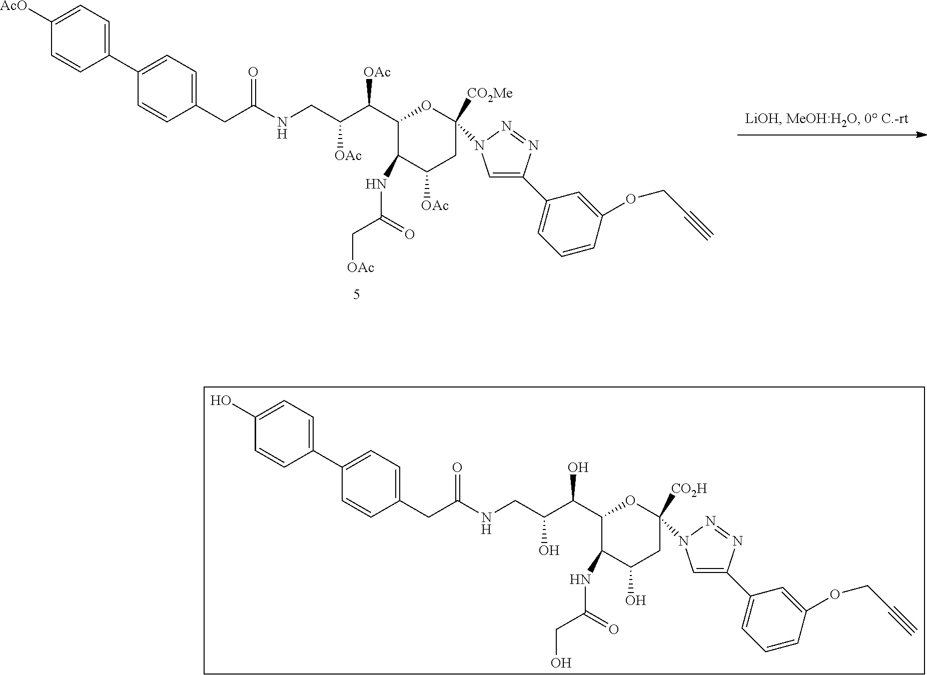 [SMILES: C#CCOc1cccc(-c2cn([C@]3(C(=O)OC)C[C@H](OC(C)=O)[C@@H](NC(=O)COC(C)=O)[C@H]([C@H](OC(C)=O)[C@@H](CNC(=O)Cc4ccc(-c5ccc(OC(C)=O)cc5)cc4)OC(C)=O)O3)nn2)c1 ; C#CCOc1cccc(-c2cn([C@]3(C(=O)O)C[C@H](O)[C@@H](NC(=O)CO)[C@H]([C@H](O)[C@H](O)CNC(=O)Cc4ccc(-c5ccc(O)cc5)cc4)O3)nn2)c1]

### Synthesis of (1R,2R)-1-((2R,3R,4S,6R)-4-acetoxy-3-(2-acetoxyacetamido)-6-(4-(3-hydroxyphenyl)-1H-1,2,3-triazol-1-yl)-6-(methoxycarbonyl)tetrahydro-2H-pyran-2-yl)-3-(2-(4′-acetoxy-[1,1′-biphenyl]-4-yl)acetamido)propane-1,2-diyl diacetate (3)

To a stirred solution of (1R,2R)-1-((2R,3R,4S,6R)-4-acetoxy-3-(2-acetoxyacetamido)-6-azido-6-(methoxycarbonyl)tetrahydro-2H-pyran-2-yl)-3-(2-(4′-acetoxy-[1,1′-biphenyl]-4-yl)acetamido)propane-1,2-diyl diacetate (0.30 g, 0.383 mmol) in dimethyl sulfoxide (5 mL) were added successively 3-ethynylphenol (2, 0.136 g, 1.15 mmol), 1 M copper(2+) pentahydrate sulfate solution (5 ml) and 1 M sodium 5-[(1S)-1,2-dihydroxyethyl]-3-hydroxy-2,4-dioxooxolan-3-ide (5 ml) aqueous solution. The reaction mixture was stirred for 12 h at room temperature. The reaction mixture was diluted with ethyl acetate and cold water. The organic layer was washed with brine (30 mL) and the combined extracts were concentrated. The crude residue obtained was purified by silica gel column chromatography using ethyl acetate and heptane as eluent to afford the desired product. (0.2 g; Y:70%); LCMS m/z 902.9 [M+H]+;

### Synthesis of (1R,2R)-1-((2R,3R,4S,6R)-4-acetoxy-3-(2-acetoxyacetamido)-6-(methoxycarbonyl)-6-(4-(3-(prop-2-yn-1-yloxy)phenyl)-1H-1,2,3-triazol-1-yl)tetrahydro-2H-pyran-2-yl)-3-(2-(4′-acetoxy-[1,1′-biphenyl]-4-yl)acetamido)propane-1,2-diyl diacetate (5)

To a solution of (1R,2R)-1-((2R,3R,4S,6R)-4-acetoxy-3-(2-acetoxyacetamido)-6-(4-(3-hydroxyphenyl)-1H-1,2,3-triazol-1-yl)-6-(methoxycarbonyl)tetrahydro-2H-pyran-2-yl)-3-(2-(4′-acetoxy-[1,1′-biphenyl]-4-yl)acetamido)propane-1,2-diyl diacetate (3, 0.25 g, 0.27 mmol) in dimethylformamide (5.0 mL), were added dipotassium carbonate (0.076 g, 0.554 mmol) and potassium iodide (0.004 g, 0.027 mmol) and the reaction mixture was heated at 70° C. for 1 h. Then 3-bromoprop-1-yne (66.0 mg, 0.55 mmol) was added dropwise to the reaction which was stirred at room temperature for another 12 h. The reaction mixture was diluted with ethyl acetate and washed with water. The organic layer was dried, filtered, and concentrated. The crude residue obtained was purified by silica gel column chromatography using ethyl acetate and heptane as eluent to afford the desired product. (0.12 g; Y:48%). LCMS m/z 939.9 [M+H]+.

### Synthesis of (2R,4S,5R,6R)-6-((1R,2R)-1,2-dihydroxy-3-(2-(4′-hydroxy-[1,1′-biphenyl]-4-yl)acetamido)propyl)-4-hydroxy-5-(2-hydroxyacetamido)-2-(4-(3-(prop-2-yn-1-yloxy)phenyl)-1H-1,2,3-triazol-1-yl)tetrahydro-2H-pyran-2-carboxylic acid (Compound 60)

To a stirred solution of (1R,2R)-1-((2R,3R,4S,6R)-4-acetoxy-3-(2-acetoxyacetamido)-6-(methoxycarbonyl)-6-(4-(3-(prop-2-yn-1-yloxy)phenyl)-1H-1,2,3-triazol-1-yl)tetrahydro-2H-pyran-2-yl)-3-(2-(4′-acetoxy-[1,1′-biphenyl]-4-yl)acetamido)propane-1,2-diyl diacetate (5, 0.10 g, 0.11 mmol) in methanol (5.0 mL), was added a solution of lithium hydroxide monohydrate (0.015 g, 0.64 mmol) in water (0.5 mL). The reaction mixture was stirred at room temperature for 6 h. The reaction mixture was treated with acidic resin (Dowex 50H⁺) to reach pH ˜6 and the suspension was filtered. The filtrate was concentrated, and the crude residue obtained was purified by prep HPLC using acetonitrile and water (+0.1% TFA) as eluent to afford the desired product. Yield: 0.016 g, 21%; LCMS m/z 716.3 [M+H]⁺; ¹H NMR (400 MHz, methanol-d₄) δ ¹H NMR (400 MHz, Methanol-d₄) δ 8.67 (s, 1H), 7.49-7.30 (m, 9H), 6.98 (dd, J=8.4 & 2.8 Hz, 1H), 6.82 (d, J=8.4 Hz, 2H), 4.77 (d, J=2.4 Hz, 2H), 4.21-3.92 (m, 6 h), 3.69 (dd, J=14.0 & 2.8 Hz, 1H), 3.56 (s, 2H), 3.47 (d, J=8.0 Hz, 1H), 3.39-3.35 (m, 1H), 3.28-3.25 (m, 1H), 2.95 (t, J=2.4 Hz, 1H), 2.26 (t, J=12.0 Hz, 1H).

### (2R,4S,5R,6R)-6-((1R,2R)-1,2-dihydroxy-3-(2-(4′-hydroxy-[1,1′-biphenyl]-4-yl)acetamido)propyl)-4-hydroxy-5-(2-hydroxyacetamido)-2-(4-(2-(prop-2-yn-1-yloxy)phenyl)-1H-1,2,3-triazol-1-yl)tetrahydro-2H-pyran-2-carboxylic acid (Compound 59)

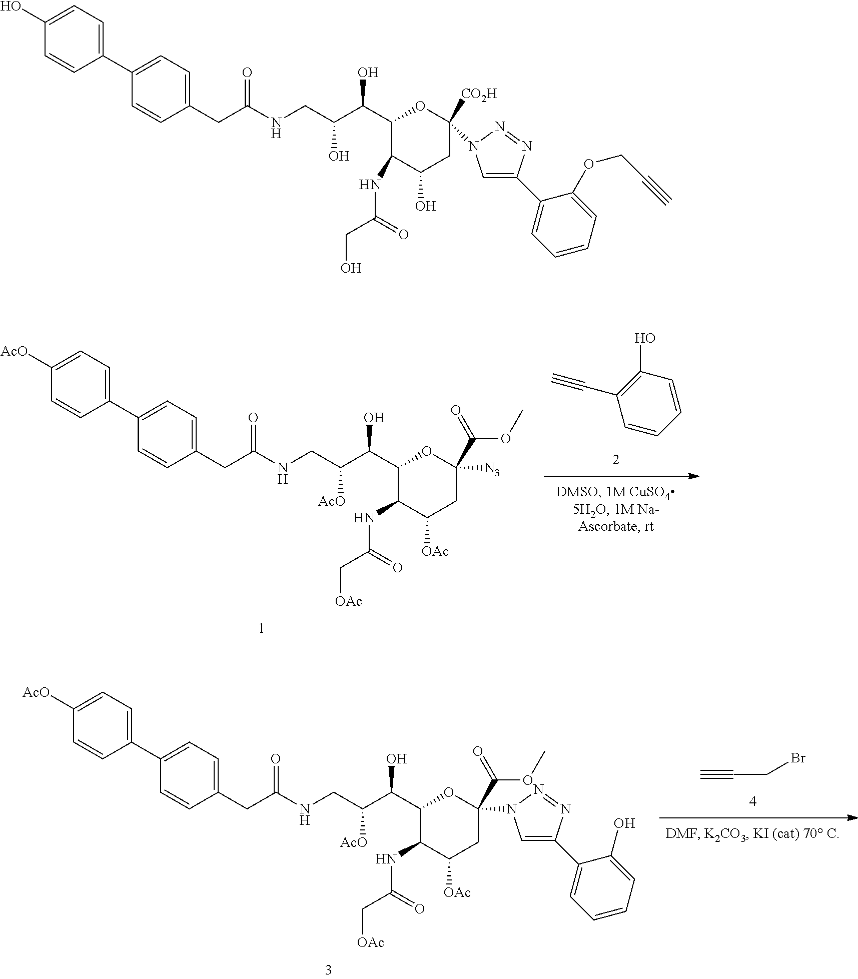

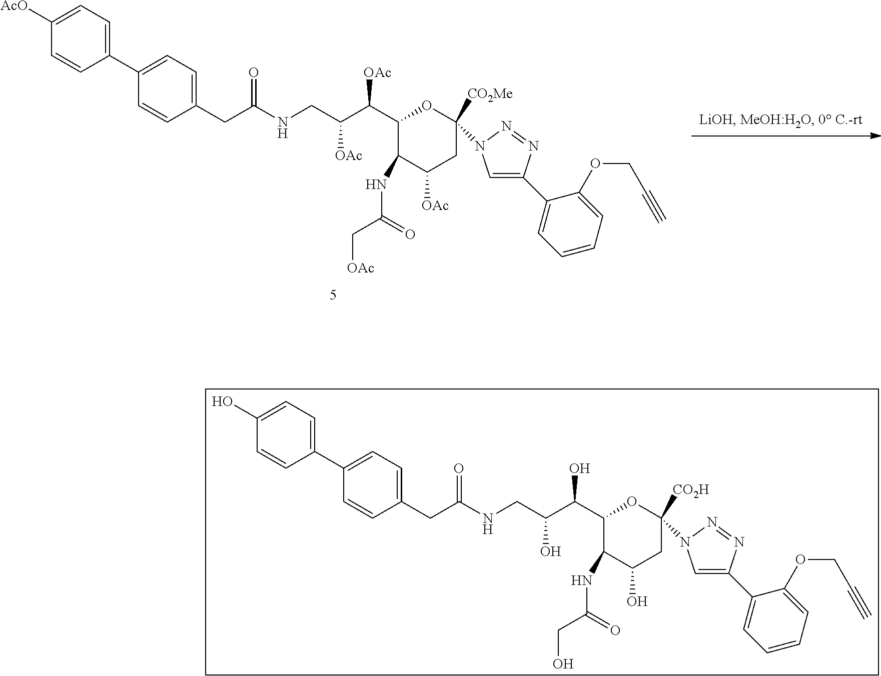 [SMILES: C#CCOc1ccccc1-c1cn([C@]2(C(=O)OC)C[C@H](OC(C)=O)[C@@H](NC(=O)COC(C)=O)[C@H]([C@H](OC(C)=O)[C@@H](CNC(=O)Cc3ccc(-c4ccc(OC(C)=O)cc4)cc3)OC(C)=O)O2)nn1 ; C#CCOc1ccccc1-c1cn([C@]2(C(=O)O)C[C@H](O)[C@@H](NC(=O)CO)[C@H]([C@H](O)[C@H](O)CNC(=O)Cc3ccc(-c4ccc(O)cc4)cc3)O2)nn1]

### Synthesis of (1R,2R)-1-((2R,3R,4S,6R)-4-acetoxy-3-(2-acetoxyacetamido)-6-(4-(2-hydroxyphenyl)-1H-1,2,3-triazol-1-yl)-6-(methoxycarbonyl)tetrahydro-2H-pyran-2-yl)-3-(2-(4′-acetoxy-[1,1′-biphenyl]-4-yl)acetamido)propane-1,2-diyl diacetate (3)

To a stirred solution of (1R,2R)-1-((2R,3R,4S,6R)-4-acetoxy-3-(2-acetoxyacetamido)-6-azido-6-(methoxycarbonyl)tetrahydro-2H-pyran-2-yl)-3-(2-(4′-acetoxy-[1,1′-biphenyl]-4-yl)acetamido)propane-1,2-diyl diacetate (0.30 g, 0.383 mmol) in dimethyl sulfoxide (5 mL) were added sequentially 2-ethynylphenol (2, 0.054 g, 0.459 mmol), 1 M copper(II) pentahydrate sulfate solution (5 ml) and 1 M sodium 5-[(1S)-1,2-dihydroxyethyl]-3-hydroxy-2,4-dioxooxolan-3-ide (5 ml) solution. The reaction mixture was stirred for 12 h at room temperature. The reaction mixture was diluted with ethyl acetate and cold water. The organic layer was washed with brine (30 mL). The combined extracts were concentrated. The crude residue obtained was purified by silica gel column chromatography using ethyl acetate and heptane as eluent to afford the desired product. (0.2 g; Y:55%); LCMS m/z 902.9 [M+H]+;

### Synthesis of (1R,2R)-1-((2R,3R,4S,6R)-4-acetoxy-3-(2-acetoxyacetamido)-6-(methoxycarbonyl)-6-(4-(2-(prop-2-yn-1-yloxy)phenyl)-1H-1,2,3-triazol-1-yl)tetrahydro-2H-pyran-2-yl)-3-(2-(4′-acetoxy-[1,1′-biphenyl]-4-yl)acetamido)propane-1,2-diyl diacetate (5)

To a solution of (1R,2R)-1-((2R,3R,4S,6R)-4-acetoxy-3-(2-acetoxyacetamido)-6-(4-(2-hydroxyphenyl)-1H-1,2,3-triazol-1-yl)-6-(methoxycarbonyl)tetrahydro-2H-pyran-2-yl)-3-(2-(4′-acetoxy-[1,1′-biphenyl]-4-yl)acetamido)propane-1,2-diyl diacetate (3, 0.25 g, 0.27 mmol) in dimethylformamide (5.0 mL), were added dipotassium carbonate (0.076 g, 0.554 mmol) and potassium iodide (0.004 g, 0.027 mmol) and the reaction mixture was heated at 70° C. for 1 h. Then 3-bromoprop-1-yne (66.0 mg, 0.55 mmol) was added dropwise to the reaction mixture which was stirred at room temperature for another 12 h. The reaction mixture was diluted with ethyl acetate and washed with water. The organic layer was dried, filtered, and concentrated. The crude residue obtained was purified by silica gel column chromatography using ethyl acetate and heptane as eluent to afford the desired product. (0.17 g; Y:65%). LCMS m/z 939.90 [M+H]+.

### Synthesis of (2R,4S,5R,6R)-6-((1R,2R)-1,2-dihydroxy-3-(2-(4′-hydroxy-[1,1′-biphenyl]-4-yl)acetamido)propyl)-4-hydroxy-5-(2-hydroxyacetamido)-2-(4-(2-(prop-2-yn-1-yloxy)phenyl)-1H-1,2,3-triazol-1-yl)tetrahydro-2H-pyran-2-carboxylic acid (Compound 59)

To a stirred solution of (1R,2R)-1-((2R,3R,4S,6R)-4-acetoxy-3-(2-acetoxyacetamido)-6-(methoxycarbonyl)-6-(4-(2-(prop-2-yn-1-yloxy)phenyl)-1H-1,2,3-triazol-1-yl)tetrahydro-2H-pyran-2-yl)-3-(2-(4′-acetoxy-[1,1′-biphenyl]-4-yl)acetamido)propane-1,2-diyl diacetate (5, 0.15 g, 0.154 mmol) in methanol (5.0 mL), was added a solution of lithium hydroxide monohydrate (0.022 g, 0.64 mmol) in water (0.5 mL). The reaction mixture was stirred at room temperature for 6 h. The reaction mixture was treated with acidic resin (Dowex 50H⁺) to reach pH ˜6 and the suspension was filtered. The filtrate was concentrated, and the crude residue obtained was purified by prep HPLC using acetonitrile and water (+0.1% TFA) as eluent to afford the desired product. Yield: 0.042 g, 38%; LCMS m/z 716.30 [M+H]⁺; ¹H NMR (400 MHz, methanol-d₄)¹H NMR (400 MHz, Methanol-d₄) δ 8.58 (s, 1H), 8.13 (dd, J=7.6 & 1.6 Hz, 1H), 7.47 (d, J=8.0 Hz, 2H), 7.41 (d, J=7.2 Hz, 2H) 7.33-7.30 (m, 3H), 7.15-7.07 (m, 2H), 6.83 (d, J=8.8 Hz, 2H), 4.82 (d, J=2.0 Hz, 2H), 4.21-3.93 (m, 6 h), 3.69 (dd, J=13.6 & 3.2 Hz, 1H), 3.56 (s, 2H), 3.46 (d, J=8.8 Hz, 1H), 3.42-3.37 (m, 1H), 3.28-3.25 (m, 1H), 3.02 (t, J=2.4 Hz, 1H), 2.35 (t, J=12.0 Hz, 1H).

### (2R,4S,5R,6R)-6-((1R,2R)-1,2-dihydroxy-3-(2-(4′-hydroxy-[1,1′-biphenyl]-4-yl)acetamido)propyl)-4-hydroxy-5-(2-hydroxyacetamido)-2-((6-(prop-2-yn-1-yloxy)hexyl)oxy)tetrahydro-2H-pyran-2-carboxylic acid (Compound 63)

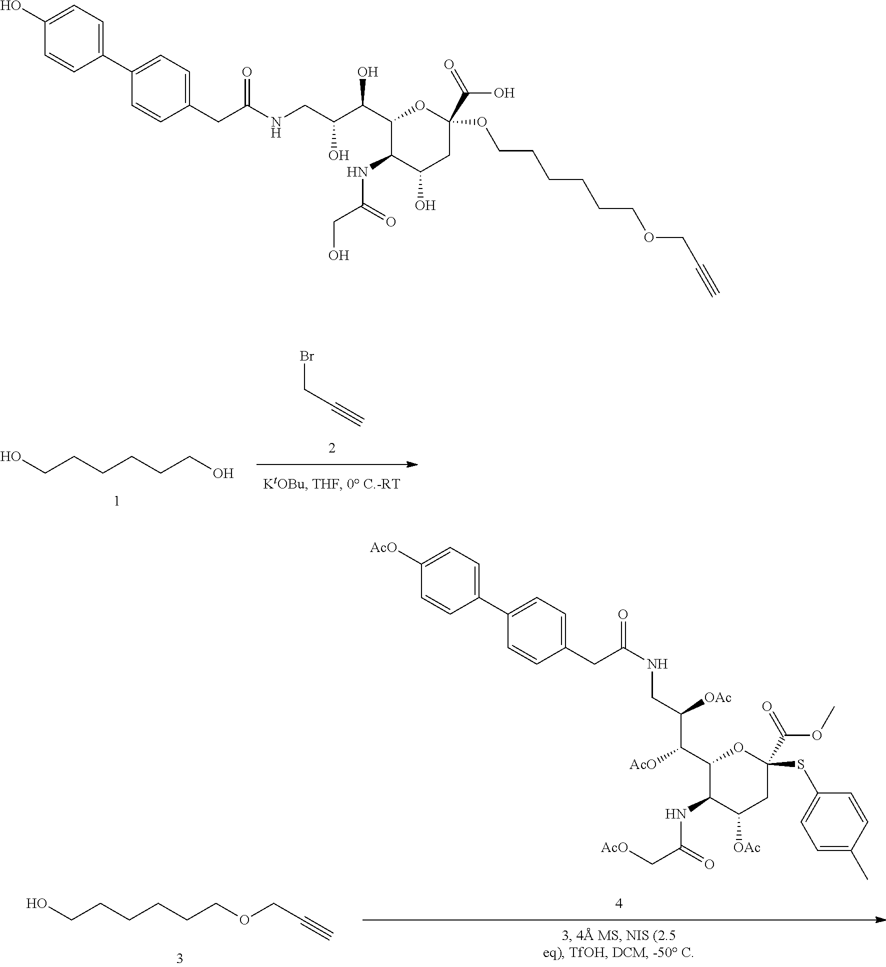

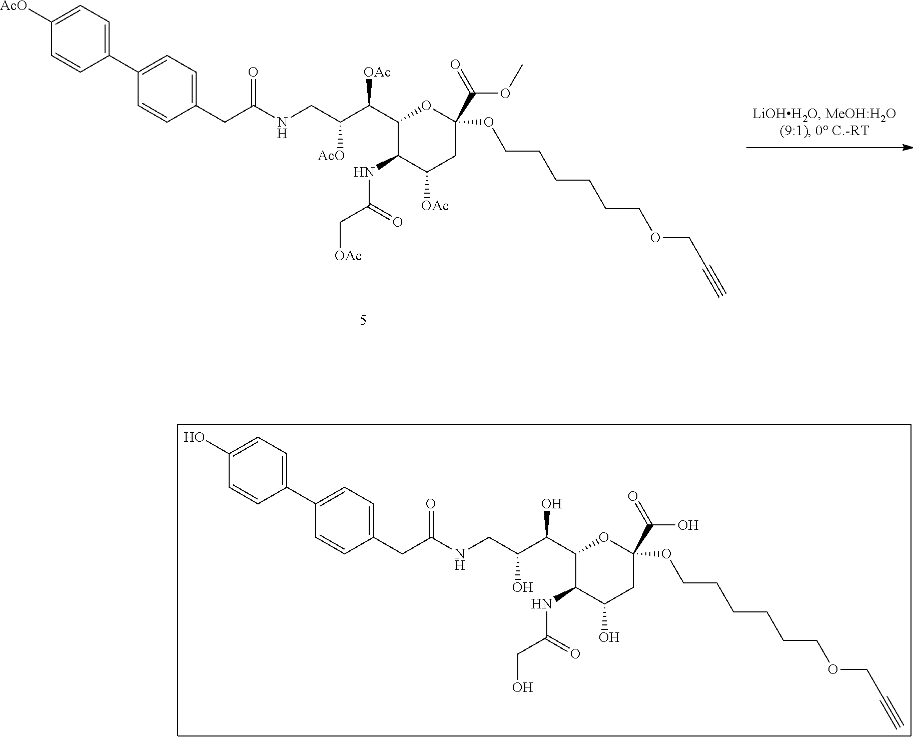 [SMILES: C#CCOCCCCCCO[C@]1(C(=O)OC)C[C@H](OC(C)=O)[C@@H](NC(=O)COC(C)=O)[C@H]([C@H](OC(C)=O)[C@@H](CNC(=O)Cc2ccc(-c3ccc(OC(C)=O)cc3)cc2)OC(C)=O)O1 ; C#CCOCCCCCCO[C@]1(C(=O)O)C[C@H](O)[C@@H](NC(=O)CO)[C@H]([C@H](O)[C@H](O)CNC(=O)Cc2ccc(-c3ccc(O)cc3)cc2)O1]

### Synthesis of 6-(prop-2-yn-1-yloxy)hexan-1-ol (3)

To a stirred solution of potassium tert-butoxide (2.37 g, 21.20 mmol) in dry tetrahydrofuran (100 mL) was added hexane-1,6-diol (5.0 g, 42.30 mmol) at 0° C. under nitrogen atmosphere. The reaction mixture was allowed to stir at room temperature for 30 minutes then 3-bromoprop-1-yne (1.60 mL, 21.20 mmol) in dry tetrahydrofuran (1.0 mL) was added dropwise. The resulting mixture was allowed to stir at room temperature for 12 h. After completion the reaction mixture was diluted with tetrahydrofuran and filtered through celite. The filtrate was concentrated, and the residue obtained was purified by column chromatography using ethyl acetate and heptane as eluent to afford the desired product. Yield: 4.0 g, 60.52%; ¹H NMR (400 MHz, DMSO-d₆) δ 4.32 (q, J=4.4 Hz, 1H), 4.08 (d, J=2.0 Hz, 1H), 3.97 (t, J=6.4 Hz, 1H), 3.42-3.32 (m, 4H), 1.99 (s, 1H), 1.51 (dt, J=6.4 & 20.4 Hz, 2H), 1.40 (t, J=6.0 Hz, 2H), 1.28 (d, J=3.2 Hz, 4H).

### Synthesis of (1R,2R)-1-((2R,3R,4S,6R)-4-acetoxy-3-(2-acetoxyacetamido)-6-(methoxycarbonyl)-6-((6-(prop-2-yn-1-yloxy)hexyl)oxy)tetrahydro-2H-pyran-2-yl)-3-(2-(4′-acetoxy-[1,1′-biphenyl]-4-yl)acetamido)propane-1,2-diyl diacetate (5)

A solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4′-acetoxy-[1,1′-biphenyl]-4-yl)acetamido)propane-1,2-diyl diacetate (0.50 g, 0.578 mmol), 6-(prop-2-yn-1-yloxy)hexan-1-ol (0.270 g, 1.73 mmol) and activated 4 Å powdered molecular sieves (2.0 g) in anhydrous dichloromethane (10.0 mL) was stirred at room temperature for 15 h under nitrogen atmosphere. 1-Iodopyrrolidine-2,5-dione (0.325 g, 1.45 mmol) and trifluoromethanesulfonic acid (0.086 mL, 0.578 mmol) were successively added at −50° C. and stirring was continued at the same temperature for 1 h. After completion the reaction mixture was quenched with triethyl amine (0.5 mL) and warmed to room temperature. The reaction mixture was filtered, and the filtrate was washed with a saturated solution of sodium bicarbonate and dried over sodium sulfate, filtered and concentrated. The crude was purified by column chromatography using ethyl acetate and hexane as eluent to afford the desired product. Yield: 0.43 g, 82%; LCMS (ESI) m/z 897.10 [M+H]+.

### Synthesis of (2R,4S,5R,6R)-6-((1R,2R)-1,2-dihydroxy-3-(2-(4′-hydroxy-[1,1′-biphenyl]-4-yl)acetamido)propyl)-4-hydroxy-5-(2-hydroxyacetamido)-2-((6-(prop-2-yn-1-yloxy)hexyl)oxy)tetrahydro-2H-pyran-2-carboxylic acid (Compound 63)

To a stirred solution of (1R,2R)-1-((2R,3R,4S,6R)-4-acetoxy-3-(2-acetoxyacetamido)-6-(methoxycarbonyl)-6-((6-(prop-2-yn-1-yloxy)hexyl)oxy)tetrahydro-2H-pyran-2-yl)-3-(2-(4′-acetoxy-[1,1′-biphenyl]-4-yl)acetamido)propane-1,2-diyl diacetate (0.40 g, 0.446 mmol) in methanol (5.0 ml), was added a solution of lithium hydroxide monohydrate (0.112 g, 2.68 mmol) in water (0.50 ml). The reaction mixture was stirred at room temperature for 6 h. After completion, the reaction mixture was treated with Dowex 50 (H+) to reach pH ˜6 and the suspension was filtered and washed with methanol. The filtrate was concentrated. The crude was purified by prep HPLC using water and acetonitrile (+0.1% TFA) as eluent to afford the desired product. Yield: 0.016 g, 5.33%; LCMS (ESI) m/z 673.45 [M+H]⁺ ¹H NMR (400 MHz, methanol-d₄) δ 6.68 (d, J=8.0 Hz, 2H), 6.62 (d, J=8.8 Hz, 2H), 6.51 (d, J=8.0 Hz, 2H), 6.02 (d, J=8.4 Hz, 2H), 3.29 (d, J=2.4 Hz, 2H), 3.20 (s, 2H), 3.08-3.04 (m, 1H), 3.01-2.83 (m, 5H), 2.75 (s, 2H), 2.67 (t, J=6.4 Hz, 2H), 2.60-2.55 (m, 2H), 2.46-2.40 (m, 1H), 1.98 (t, J=2.4 Hz, 1H), 1.92 (dd, J=3.6 & 12.4 Hz, 1H), 0.88 (t, J=12.0 Hz, 1H), 0.73-0.70 (m, 4H), 0.55-0.52 (m, 4H).

### (2R,4S,5R,6R)-2-((4-(but-3-yn-1-yloxy)benzyl)oxy)-6-((1R,2R)-1,2-dihydroxy-3-(2-(4′-hydroxy-[1,1′-biphenyl]-4-yl)acetamido)propyl)-4-hydroxy-5-(3-methylureido)tetrahydro-2H-pyran-2-carboxylic acid (Compound 61)

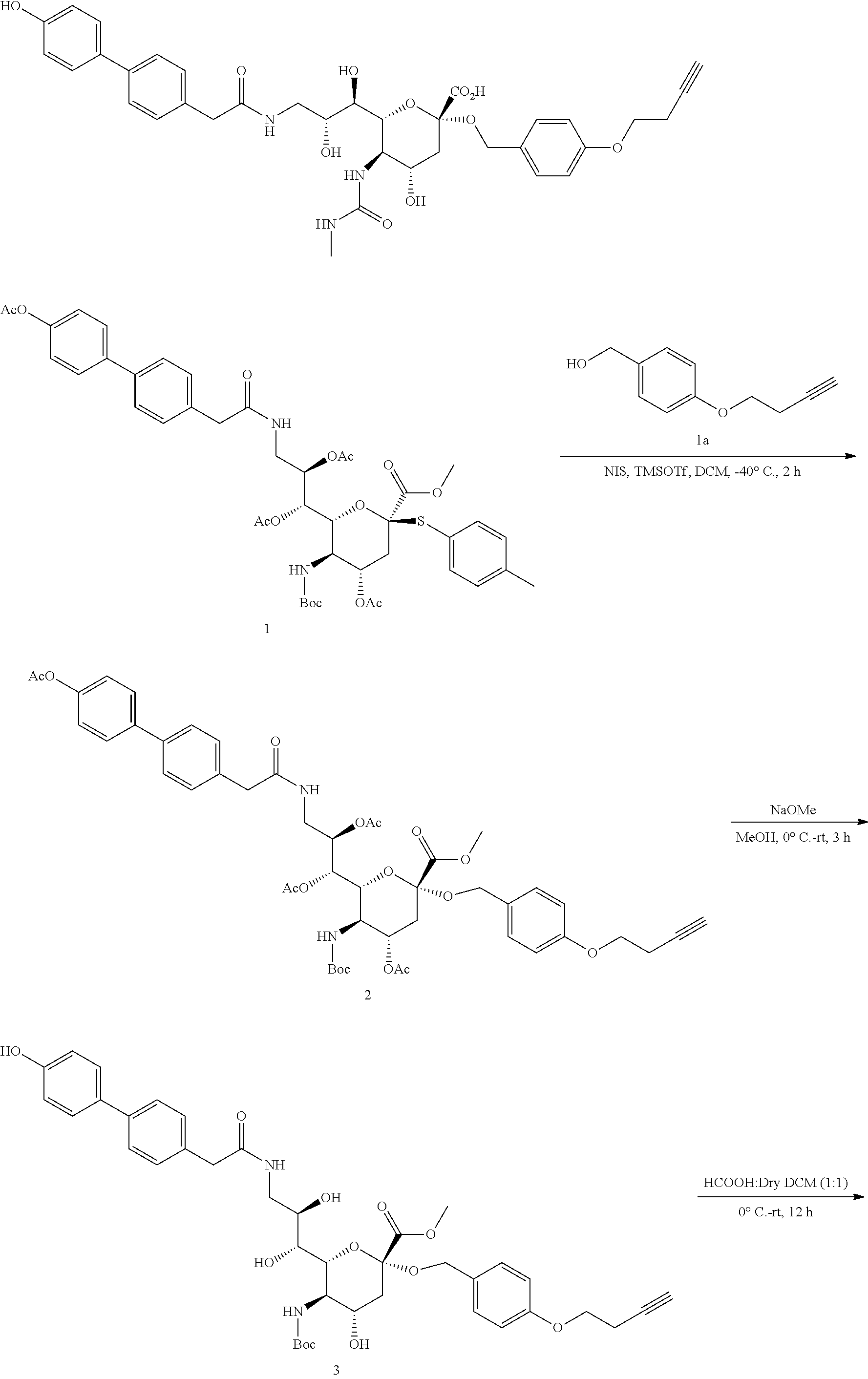

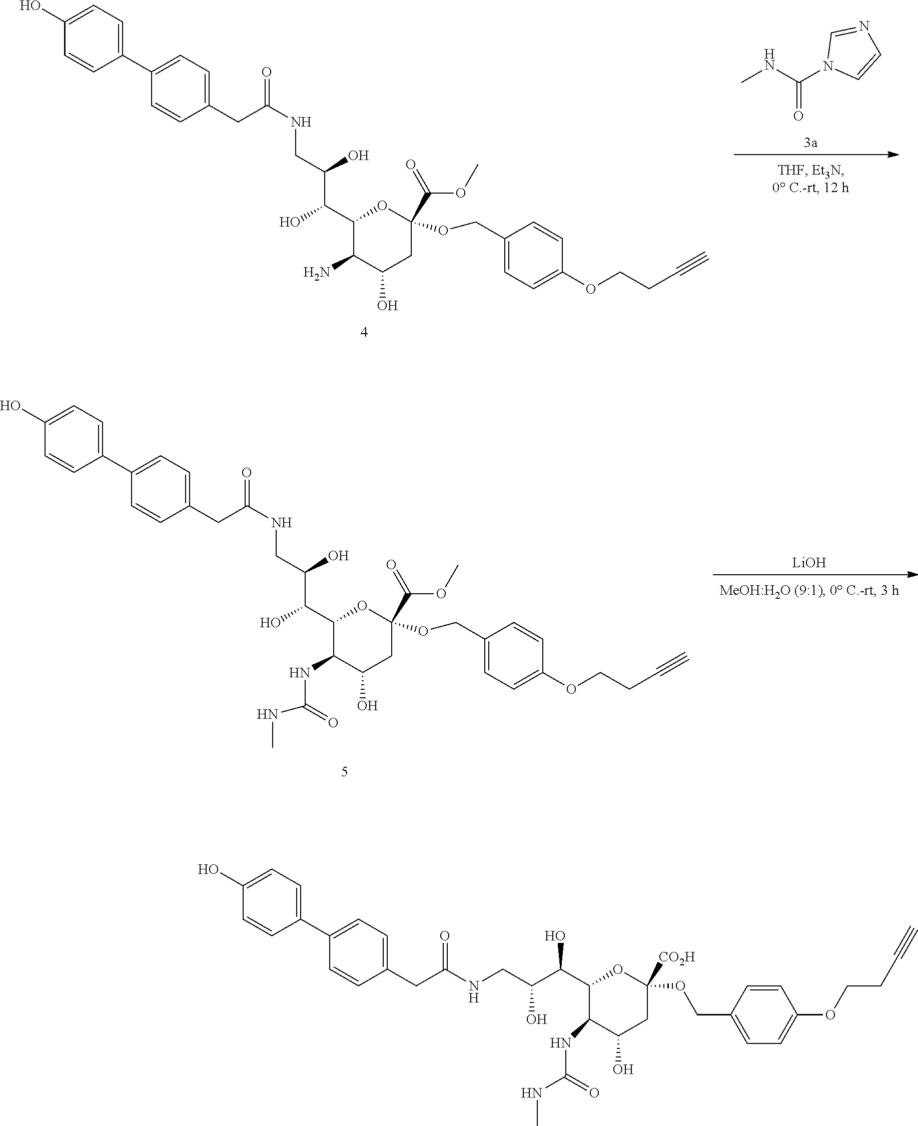

### Synthesis of (1R,2R)-1-((2R,3R,4S,6R)-4-acetoxy-6-((4-(but-3-yn-1-yloxy)benzyl)oxy)-3-((tert-butoxycarbonyl)amino)-6-(methoxycarbonyl)tetrahydro-2H-pyran-2-yl)-3-(2-(4′-acetoxy-[1,1′-biphenyl]-4-yl)acetamido)propane-1,2-diyl diacetate (2)

A suspension of (1R,2R)-1-((2R,3R,4S,6R)-4-acetoxy-3-((tert-butoxycarbonyl)amino)-6-(methoxycarbonyl)-6-(p-tolylthio)tetrahydro-2H-pyran-2-yl)-3-(2-(4′-acetoxy-[1,1′-biphenyl]-4-yl)acetamido)propane-1,2-diyl diacetate (0.70 g, 0.81 mmol), (1a, 4-(but-3-yn-1-yloxy)phenyl)methanol (0.713 g, 4.05 mmol) and activated 4 Å powdered molecular sieves (1.0 g) in anhydrous dichloromethane (14.0 mL) was stirred for 15 h at room temperature. The reaction mixture was cooled to −40° C. 1-Iodopyrrolidine-2,5-dione (0.45 g, 2.02 mmol) and trimethylsilyl trifluoromethanesulfonate (0.17 mL, 0.97 mmol) were added and the reaction was stirred at −40° C. for 2 h. After completion, the reaction mixture was quenched with triethylamine (0.2 mL) and warmed to room temperature. The reaction mixture was filtered then washed with dichloromethane. The filtrate was washed with a saturated solution of sodium thiosulfate and dried over sodium sulfate, filtered and concentrated. The crude product was purified by column chromatography using ethyl acetate and heptane as eluent to afford the desired product. Yield: 0.4 g, 54%; LCMS (ESI) m/z 917.05 [M+H]+.

### Synthesis of methyl (2R,4S,5R,6R)-2-((4-(but-3-yn-1-yloxy)benzyl)oxy)-5-((tert-butoxycarbonyl)amino)-6-((1R,2R)-1,2-dihydroxy-3-(2-(4′-hydroxy-[1,1′-biphenyl]-4-yl)acetamido)propyl)-4-hydroxytetrahydro-2H-pyran-2-carboxylate (3)

(1R,2R)-1-((2R,3R,4S,6R)-4-acetoxy-6-((4-(but-3-yn-1-yloxy)benzyl)oxy)-3-((tert-butoxycarbonyl)amino)-6-(methoxycarbonyl)tetrahydro-2H-pyran-2-yl)-3-(2-(4′-acetoxy-[1,1′-biphenyl]-4-yl)acetamido)propane-1,2-diyl diacetate (2, 0.1 g, 0.10 mmol) was dissolved in MeOH (5 mL) and 25% NaOMe in methanol (0.01 mL, 0.010 mmol) was added at 0° C. The reaction mixture was stirred for 1 h at room temperature. The solution was neutralized with Dowex-50-Hydrogen to reach neutral pH, filtered, washed with methanol and the filtrate was concentrated. The crude was triturated with ether and dried under vacuum to afford the desired product. Yield: 0.080 g; LCMS (ESI) m/z 749.15 [M+H]+.

### Synthesis of methyl (2R,4S,5R,6R)-5-amino-2-((4-(but-3-yn-1-yloxy)benzyl)oxy)-6-((1R,2R)-1,2-dihydroxy-3-(2-(4′-hydroxy-[1,1′-biphenyl]-4-yl)acetamido)propyl)-4-hydroxytetrahydro-2H-pyran-2-carboxylate (4)

Methyl (2R,4S,5R,6R)-2-((4-(but-3-yn-1-yloxy)benzyl)oxy)-5-((tert-butoxycarbonyl)amino)-6-((1R,2R)-1,2-dihydroxy-3-(2-(4′-hydroxy-[1,1′-biphenyl]-4-yl)acetamido)propyl)-4-hydroxytetrahydro-2H-pyran-2-carboxylate (3, 0.080 g, 0.107 mmol) was dissolved in anhydrous dichloromethane (1 mL) and cooled to 0° C. Formic acid (1 mL) was slowly added dropwise to the solution and stirred for 10 min at 0° C. Ice bath was removed and the mixture was stirred for 12 h at room temperature. The reaction mixture was concentrated and co-evaporated with dichloromethane 3 times. The residue obtained was co-evaporated with diethyl ether, washed with diethyl ether and dried to afford the desired product. Yield: 0.080 g, Crude; LCMS (ESI) m/z 649.10 [M+H]+.

### Synthesis of methyl (2R,4S,5R,6R)-2-((4-(but-3-yn-1-yloxy)benzyl)oxy)-6-((1R,2R)-1,2-dihydroxy-3-(2-(4′-hydroxy-[1,1′-biphenyl]-4-yl)acetamido)propyl)-4-hydroxy-5-(3-methylureido)tetrahydro-2H-pyran-2-carboxylate (5)

To a solution of methyl (2R,4S,5R,6R)-5-amino-2-((4-(but-3-yn-1-yloxy)benzyl)oxy)-6-((1R,2R)-1,2-dihydroxy-3-(2-(4′-hydroxy-[1,1′-biphenyl]-4-yl)acetamido)propyl)-4-hydroxytetrahydro-2H-pyran-2-carboxylate (4, 0.080 g, 0.123 mmol) and N-methyl-1H-imidazole-1-carboxamide (3a, 0.015 g, 0.123 mmol) in tetrahydrofuran (2 mL), was added triethylamine (0.068 mL, 0.493 mmol) at 0° C. and the reaction mixture was stirred at room temperature for 12 h. The reaction mixture was concentrated, washed with diethyl ether and dried to afford the desired product (crude). Yield: 0.060 g; LCMS (ESI) m/z 706.15 [M+H]+.

### Synthesis of (2R,4S,5R,6R)-2-((4-(but-3-yn-1-yloxy)benzyl)oxy)-6-((1R,2R)-1,2-dihydroxy-3-(2-(4′-hydroxy-[1,1′-biphenyl]-4-yl)acetamido)propyl)-4-hydroxy-5-(3-methylureido)tetrahydro-2H-pyran-2-carboxylic acid (Compound 61)

To a solution of methyl (2R,4S,5R,6R)-2-((4-(but-3-yn-1-yloxy)benzyl)oxy)-6-((1R,2R)-1,2-dihydroxy-3-(2-(4′-hydroxy-[1,1′-biphenyl]-4-yl)acetamido)propyl)-4-hydroxy-5-(3-methylureido)tetrahydro-2H-pyran-2-carboxylate (5, 0.060 g, 0.085 mmol) in methanol (1 mL), was added a solution of lithium hydroxide monohydrate (0.004 g, 0.17 mmol) in water (0.1 mL). The reaction mixture was stirred at room temperature for 4 h. The reaction mixture was quenched with acidic (Dowex-50, H⁺) resin to reach pH˜7 and the suspension was filtered then washed with methanol. The filtrate was concentrated, and the crude residue obtained was purified by Prep-HPLC using acetonitrile and water as eluent to afford the desired product. Yield: 0.012 g, 20%; LCMS (ESI) m/z 692.35 [M+H]⁺; ¹H NMR (400 MHz, methanol-d₄) δ 7.45 (d, J=8.4 Hz, 2H), 7.38 (d, J=8.4 Hz, 2H), 7.31 (d, J=8.0 Hz, 2H), 7.21 (d, J=8.4 Hz, 2H), 6.83 (t, J=8.0 Hz, 4H), 4.71 (d, J=10.8 Hz, 1H), 4.39 (d, J=7.6 Hz, 1H), 4.07 (t, J=6.8 Hz, 2H), 3.97-3.92 (m, 1H), 3.70 (dd, J=13.8 & 2.4 Hz, 1H), 3.63-3.47 (m, 6 h), 3.30-3.27 (m, 1H), 2.77-2.71 (m, 1H), 2.68 (s, 3H), 2.64-2.60 (m, 2H), 2.31 (t, J=2.8 Hz, 1H), 1.78-1.72 (m, 1H).

### (2R,4S,5R,6R)-2-((4-(but-3-yn-1-yloxy)benzyl)oxy)-6-((1R,2R)-1,2-dihydroxy-3-(1-phenylcyclopropane-1-carboxamido)propyl)-4-hydroxy-5-(2-hydroxyacetamido)tetrahydro-2H-pyran-2-carboxylic acid (Compound 173)

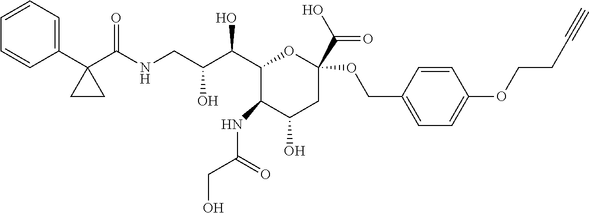 [SMILES: C#CCCOc1ccc(CO[C@]2(C(=O)O)C[C@H](O)[C@@H](NC(=O)CO)[C@H]([C@H](O)[C@H](O)CNC(=O)C3(c4ccccc4)CC3)O2)cc1]

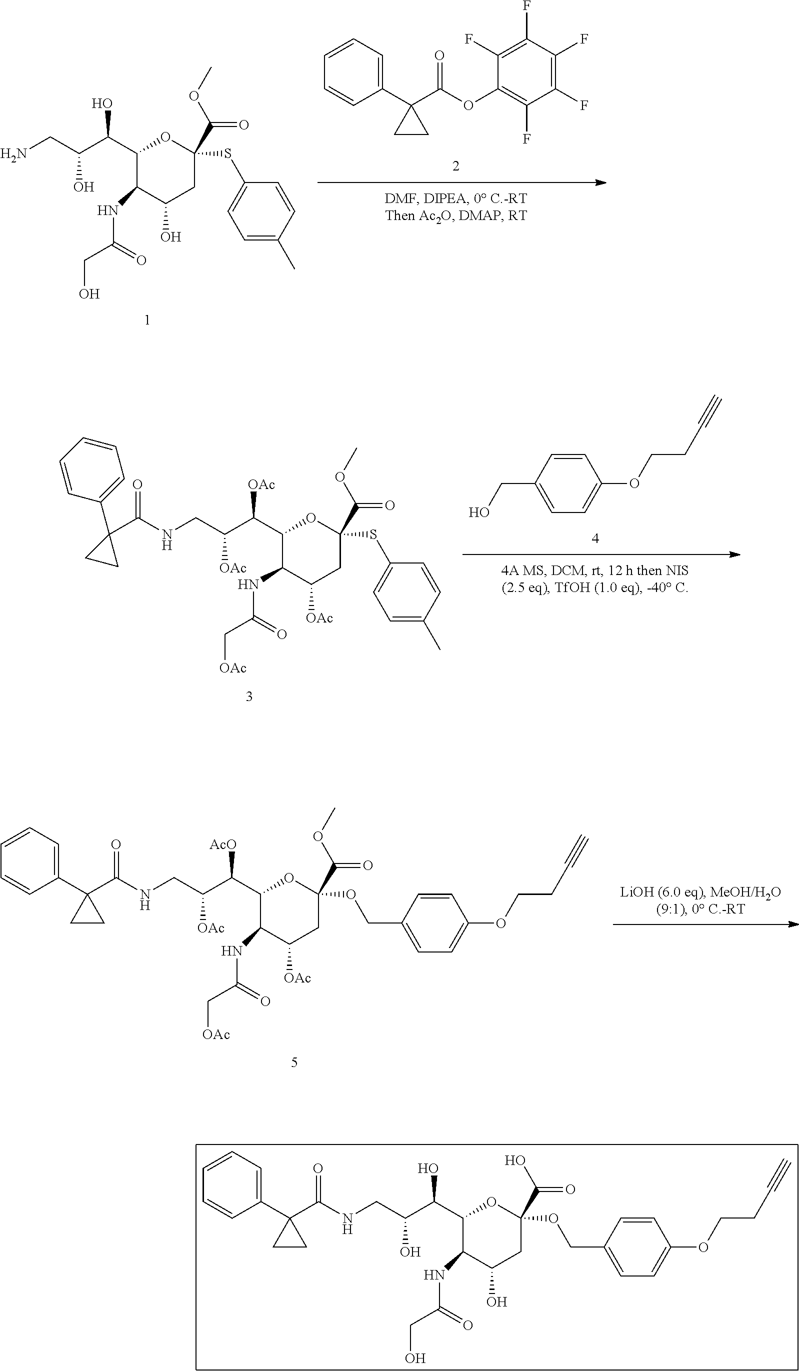

### Synthesis of (1R,2R)-1-((2R,3R,4S,6S)-4-acetoxy-3-(2-acetoxyacetamido)-6-(methoxycarbonyl)-6-(p-tolylthio)tetrahydro-2H-pyran-2-yl)-3-(1-phenylcyclopropane-1-carboxamido)propane-1,2-diyl diacetate (3)

To a stirred solution of methyl (2S,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (1.0 g, 2.25 mmol) and perfluorophenyl 1-phenylcyclopropane-1-carboxylate (2, 0.59 g, 1.8 mmol) in dimethylformamide (10.0 mL), was added DIPEA (1.96 mL, 11.20 mmol) at 0° C. and the reaction mixture was stirred at room temperature for 4 h. After completion acetic anhydride (2.0 ml) and 4-dimethylaminopyridine (0.027 g, 0.225 mmol) were added to the reaction mixture and allowed to stir for 12 h. After completion the reaction mixture was concentrated and the crude residue obtained was further washed with a saturated solution of sodium bicarbonate and dried over sodium sulfate, filtered and concentrated. The crude residue obtained was purified by flash column chromatography using ethyl acetate and heptane as eluent to afford the desired product. Yield: 0.80 g, 46%; LCMS m/z 757.35 [M+H]+.

### Synthesis of (1R,2R)-1-((2R,3R,4S,6R)-4-acetoxy-3-(2-acetoxyacetamido)-6-((4-(but-3-yn-1-yloxy)benzyl)oxy)-6-(methoxycarbonyl)tetrahydro-2H-pyran-2-yl)-3-(1-phenylcyclopropane-1-carboxamido)propane-1,2-diyl diacetate (5)

A suspension of (1R,2R)-1-((2R,3R,4S,6S)-4-acetoxy-3-(2-acetoxyacetamido)-6-(methoxycarbonyl)-6-(p-tolylthio)tetrahydro-2H-pyran-2-yl)-3-(1-phenylcyclopropane-1-carboxamido)propane-1,2-diyl diacetate (3, 0.60 g, 0.793 mmol), (4-(but-3-yn-1-yloxy)phenyl)methanol (4, 0.699 g, 3.96 mmol) and activated 4 Å powdered molecular sieves (2.00 g) in anhydrous dichloromethane (14 mL) was stirred at room temperature for 12 h under nitrogen atmosphere. N-iodosuccinimide (0.446 g, 1.98 mmol) and trifluoromethanesulfonic acid (0.070 mL, 0.793 mmol) in dichloromethane (1.0 mL) were added at −40° C. and stirred at the same temperature for 1 h. After completion the reaction mixture was quenched with triethylamine (1.0 mL) and warmed to room temperature. The reaction mixture was filtered and washed with dichloromethane. The filtrate was washed with a saturated solution of sodium bicarbonate and the organic layer was dried over sodium sulfate, filtered and concentrated. The crude product was purified by column chromatography using ethyl acetate and heptane as eluent to afford the desired product. Yield: 0.45 g, 83%; LCMS m/z 809.15 [M+H]+.

### Synthesis of (2R,4S,5R,6R)-2-((4-(but-3-yn-1-yloxy)benzyl)oxy)-6-((1R,2R)-1,2-dihydroxy-3-(1-phenylcyclopropane-1-carboxamido)propyl)-4-hydroxy-5-(2-hydroxyacetamido)tetrahydro-2H-pyran-2-carboxylic acid (Compound 173)

To a stirred solution of (1R,2R)-1-((2R,3R,4S,6R)-4-acetoxy-3-(2-acetoxyacetamido)-6-((4-(but-3-yn-1-yloxy)benzyl)oxy)-6-(methoxycarbonyl)tetrahydro-2H-pyran-2-yl)-3-(1-phenylcyclopropane-1-carboxamido)propane-1,2-diyl diacetate (5, 0.4 g, 0.495 mmol) in methanol (4.0 mL), was added a solution of lithium hydroxide monohydrate (0.125 g, 2.97 mmol)) in water (1.0 mL). The reaction mixture was stirred at room temperature for 6 h. After completion, the reaction mixture was treated with acidic resin (Dowex 50, H⁺) to reach pH ˜6 and the suspension was filtered. The filtrate was concentrated. The crude residue obtained was purified by prep HPLC using water and acetonitrile (+0.1% FA) as eluent to afford the desired product. Yield: 0.078 g, 25%; LCMS m/z 627.35 [M+H]⁺; ¹H NMR (400 MHz, methanol-d₄) δ 7.43-7.35 (m, 4H), 7.34-7.29 (m, 1H), 7.25 (d, J=8.4 Hz, 2H), 6.89 (d, J=8.4 Hz, 2H), 4.69 (d, J=11.2 Hz, 1H), 4.40 (d, J=10.8 Hz, 1H), 4.08 (t, J=6.8 Hz, 2H), 4.04 (s, 2H), 3.86-3.74 (m, 3H), 3.69-3.67 (m, 1H), 3.55-3.48 (m, 1H), 3.31-3.27 (m, 2H), 2.74 (dd, J=12.8 Hz & 4.4 Hz, 1H), 2.66-2.62 (m, 2H), 2.32 (t, J=2.8 Hz, 1H), 1.74 (t, J=12.4 Hz, 1H), 1.52-1.49 (m, 2H), 1.08-1.06 (m, 2H).

### (2R,4S,5R,6R)-2-((4-(but-3-yn-1-yloxy)benzyl)oxy)-6-((1R,2R)-3-(2,2-difluoro-2-phenylacetamido)-1,2-dihydroxypropyl)-4-hydroxy-5-(2-hydroxyacetamido)tetrahydro-2H-pyran-2-carboxylic acid (Compound 172)

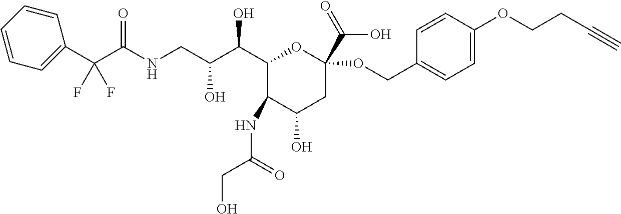 [SMILES: C#CCCOc1ccc(CO[C@]2(C(=O)O)C[C@H](O)[C@@H](NC(=O)CO)[C@H]([C@H](O)[C@H](O)CNC(=O)C(F)(F)c3ccccc3)O2)cc1]

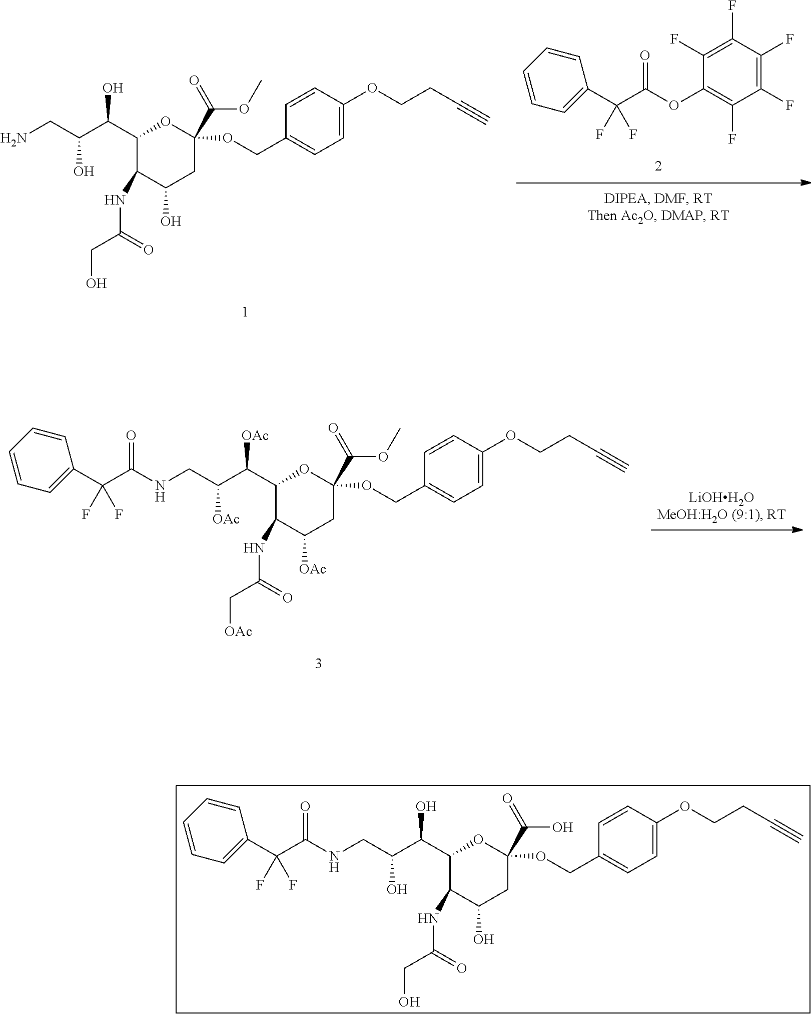 [SMILES: C#CCCOc1ccc(CO[C@]2(C(=O)OC)C[C@H](O)[C@@H](NC(=O)CO)[C@H]([C@H](O)[C@H](O)CN)O2)cc1 ; O=C(Oc1c(F)c(F)c(F)c(F)c1F)C(F)(F)c1ccccc1 ; C#CCCOc1ccc(CO[C@]2(C(=O)OC)C[C@H](OC(C)=O)[C@@H](NC(=O)COC(C)=O)[C@H]([C@H](OC(C)=O)[C@@H](CNC(=O)C(F)(F)c3ccccc3)OC(C)=O)O2)cc1 ; "LiOH·H2OMeOH:H2O (9:1), RT" ; C#CCCOc1ccc(CO[C@]2(C(=O)O)C[C@H](O)[C@@H](NC(=O)CO)[C@H]([C@H](O)[C@H](O)CNC(=O)C(F)(F)c3ccccc3)O2)cc1]

### Synthesis of (1R,2R)-1-((2R,3R,4S,6R)-4-acetoxy-3-(2-acetoxyacetamido)-6-((4-(but-3-yn-1-yloxy)benzyl)oxy)-6-(methoxycarbonyl)tetrahydro-2H-pyran-2-yl)-3-(2,2-difluoro-2-phenylacetamido)propane-1,2-diyl diacetate (3)

To a stirred solution of methyl (2R,4S,5R,6R)-6-((1R,2R)-3-amino-1,2-dihydroxypropyl)-2-((4-(but-3-yn-1-yloxy)benzyl)oxy)-4-hydroxy-5-(2-hydroxyacetamido)tetrahydro-2H-pyran-2-carboxylate (2, 0.2 g, 0.403 mmol) and perfluorophenyl 2,2-difluoro-2-phenylacetate (0.136 g 0.403 mmol) in N, N-dimethylformamide (3.0 mL), was added DIPEA (0.30 mL, 2.01 mmol) at 0° C. and the reaction mixture was stirred at room temperature for 4 h. After completion, acetic anhydride (0.30 ml, 4.03 mmol) and 4-dimethylaminopyridine (0.014 g, 1.12 mmol)) were added to the reaction mixture which was stirred for 12 h. The reaction mixture was concentrated. The residue obtained was diluted with ethyl acetate, washed with saturated solution of sodium bicarbonate, once with water, and twice with brine. The organic layers were dried over sodium sulfate, filtered and the filtrate was concentrated. The crude residue obtained was purified by flash column chromatography using ethyl acetate and heptane as eluent to afford the desired product. Yield: 0.110 g, 33%; LCMS m/z 817.05 [M−H]⁻.

### Synthesis of (2R,4S,5R,6R)-2-((4-(but-3-yn-1-yloxy)benzyl)oxy)-6-((1R,2R)-3-(2,2-difluoro-2-phenylacetamido)-1,2-dihydroxypropyl)-4-hydroxy-5-(2-hydroxyacetamido)tetrahydro-2H-pyran-2-carboxylic acid (Compound 172)

To a stirred solution of (1R,2R)-1-((2R,3R,4S,6R)-4-acetoxy-3-(2-acetoxyacetamido)-6-((4-(but-3-yn-1-yloxy)benzyl)oxy)-6-(methoxycarbonyl)tetrahydro-2H-pyran-2-yl)-3-(2,2-difluoro-2-phenylacetamido)propane-1,2-diyl diacetate (3, 0.150 g, 0.183 mmol) in methanol (2.0 mL), was added a solution of lithium hydroxide monohydrate (0.046 g, 1.100 mmol)) in water (0.20 mL). The reaction mixture was stirred at room temperature for 6 h. After completion, the reaction mixture was treated with acidic resin (Dowex 50, H⁺) to reach pH ˜6 and the suspension was filtered. The filtrate was concentrated under reduced pressure. The crude residue obtained was purified by prep HPLC using water and acetonitrile (+0.1% formic acid) as eluent to afford the desired product. Yield: 0.009 g, 8%; LCMS m/z 637.35 [M+H]⁺; ¹H NMR (400 MHz, methanol-d₄) δ 8.69-8.66 (m, 1H), 7.92 (d, J=7.2 Hz, 1H), 7.63-7.61 (m, 2H), 7.49-7.42 (m, 3H), 7.24 (d, J=8.8 Hz, 2H), 6.88 (d, J=8.4 Hz, 2H), 4.73 (d, J=10.8 Hz, 1H), 4.69 (d, J=2.4 Hz, 2H), 4.41 (d, J=10.8 Hz, 1H), 4.07 (t, J=6.8 Hz, 2H), 4.02 (s, 2H), 4.01-3.97 (m, 1H), 3.89-3.68 (m, 4H), 3.43-3.39 (m, 2H), 2.76 (dd, J=12.8 Hz & 4 Hz, 1H), 2.66-2.62 (m, 2H), 2.32 (t, J=2.8 Hz), 1.78 (t, J=12 Hz, 1H).

### (2R,4S,5R,6R)-2-((4-(but-3-yn-1-yloxy)benzyl)oxy)-6-((1R,2R)-1,2-dihydroxy-3-(3,3,3-trifluoro-2-methoxy-2-phenylpropanamido)propyl)-4-hydroxy-5-(2-hydroxyacetamido)tetrahydro-2H-pyran-2-carboxylic acid (Compound 171)

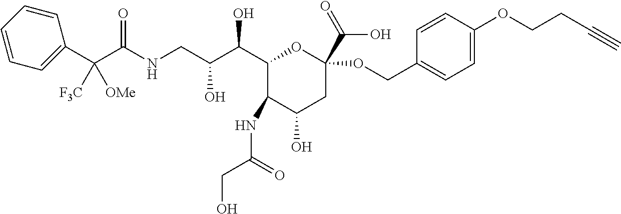 [SMILES: C#CCCOc1ccc(CO[C@]2(C(=O)O)C[C@H](O)[C@@H](NC(=O)CO)[C@H]([C@H](O)[C@H](O)CNC(=O)C(OC)(c3ccccc3)C(F)(F)F)O2)cc1]

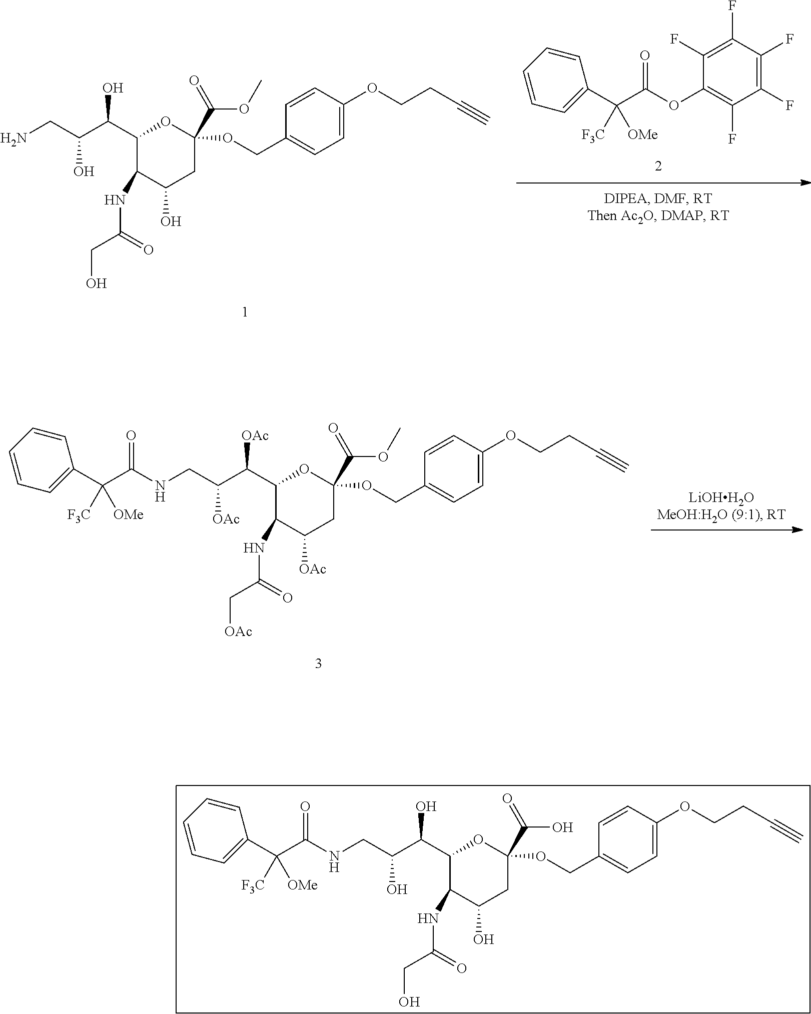 [SMILES: C#CCCOc1ccc(CO[C@]2(C(=O)OC)C[C@H](O)[C@@H](NC(=O)CO)[C@H]([C@H](O)[C@H](O)CN)O2)cc1 ; COC(C(=O)Oc1c(F)c(F)c(F)c(F)c1F)(c1ccccc1)C(F)(F)F ; C#CCCOc1ccc(CO[C@]2(C(=O)OC)C[C@H](OC(C)=O)[C@@H](NC(=O)COC(C)=O)[C@H]([C@H](OC(C)=O)[C@@H](CNC(=O)C(OC)(c3ccccc3)C(F)(F)F)OC(C)=O)O2)cc1 ; "LiOH·H2OMeOH:H2O (9:1), RT" ; C#CCCOc1ccc(CO[C@]2(C(=O)O)C[C@H](O)[C@@H](NC(=O)CO)[C@H]([C@H](O)[C@H](O)CNC(=O)C(OC)(c3ccccc3)C(F)(F)F)O2)cc1]

### Synthesis of (1R,2R)-1-((2R,3R,4S,6R)-4-acetoxy-3-(2-acetoxyacetamido)-6-((4-(but-3-yn-1-yloxy)benzyl)oxy)-6-(methoxycarbonyl)tetrahydro-2H-pyran-2-yl)-3-(3,3,3-trifluoro-2-methoxy-2-phenylpropanamido)propane-1,2-diyl diacetate (3)

To a stirred solution of methyl (2R,4S,5R,6R)-6-((1R,2R)-3-amino-1,2-dihydroxypropyl)-2-((4-(but-3-yn-1-yloxy)benzyl)oxy)-4-hydroxy-5-(2-hydroxyacetamido)tetrahydro-2H-pyran-2-carboxylate (1, 0.090 g, 0.181 mmol) and perfluorophenyl 3,3,3-trifluoro-2-methoxy-2-phenylpropanoate (2, 0.072 g, 0.181 mmol) in N,N-dimethylformamide (3.0 mL), was added DIPEA (0.15 mL, 0.90 mmol) at 0° C. and the reaction mixture was stirred at room temperature for 4 h. After completion acetic anhydride (0.20 mL, 1.81 mmol) and 4-dimethylaminopyridine (0.002 g, 0.0180 mmol) were added to the reaction mixture which was stirred for 12 h at rt. The mixture was concentrated, and the residue obtained was diluted with ethyl acetate and washed with saturated solution of sodium bicarbonate. The organic layer was dried over sodium sulfate, filtered and concentrated. The crude residue obtained was purified by flash column chromatography using ethyl acetate and heptane as eluent to afford the desired product. Yield: 0.090 g, 56%; LCMS m/z 879.00 [M−H]−.

### Synthesis of (2R,4S,5R,6R)-2-((4-(but-3-yn-1-yloxy)benzyl)oxy)-6-((1R,2R)-1,2-dihydroxy-3-(3,3,3-trifluoro-2-methoxy-2-phenylpropanamido)propyl)-4-hydroxy-5-(2-hydroxyacetamido)tetrahydro-2H-pyran-2-carboxylic acid (Compound 171)

To a stirred solution of (1R,2R)-1-((2R,3R,4S,6R)-4-acetoxy-3-(2-acetoxyacetamido)-6-((4-(but-3-yn-1-yloxy)benzyl)oxy)-6-(methoxycarbonyl)tetrahydro-2H-pyran-2-yl)-3-(3,3,3-trifluoro-2-methoxy-2-phenylpropanamido)propane-1,2-diyl diacetate (3, 0.090 g, 0.102 mmol) in methanol (2.0 mL), was added a solution of lithium hydroxide monohydrate (0.0257 g, 0.613 mmol) in water (0.20 mL). The reaction mixture was stirred at room temperature for 6 h. After completion, the reaction mixture was treated with acidic resin (Dowex 50, H⁺) to reach pH ˜6 and the suspension was filtered. The filtrate was concentrated under reduced pressure. The crude residue obtained was purified by prep HPLC using water and acetonitrile (+0.1% formic acid) as eluent to afford the desired product. Yield: 0.008 g, 11%; LCMS m/z 699.35 [M+H]⁺; ¹H NMR (400 MHz, methanol-d₄) δ 8.19 (t, J=5.6 Hz, 1H), 7.58-7.56 (m, 2H), 7.43-7.38 (m, 3H), 7.24 (d, J=8.8 Hz, 2H), 6.89-6.85 (m, 2H), 4.73 (d, J=11.2 Hz, 1H), 4.42 (d, J=10.8 Hz, 1H), 4.07 (t, J=6.8 Hz, 2H), 4.02-3.96 (m, 3H), 3.88-3.65 (m, 4H), 3.49-3.42 (m, 5H), 2.77 (dd, J=12.8 Hz & 4 Hz, 1H), 2.65-2.61 (m, 2H), 2.32 (t, J=2.6 Hz, 1H), 1.78 (t, J=12.4 Hz, 1H).

### (2R,4S,5R,6R)-2-((4-(but-3-yn-1-yloxy)benzyl)oxy)-6-((1R,2R)-1,2-dihydroxy-3-(2-hydroxy-2-phenylpropanamido)propyl)-4-hydroxy-5-(2-hydroxyacetamido)tetrahydro-2H-pyran-2-carboxylic acid (Compound 169)

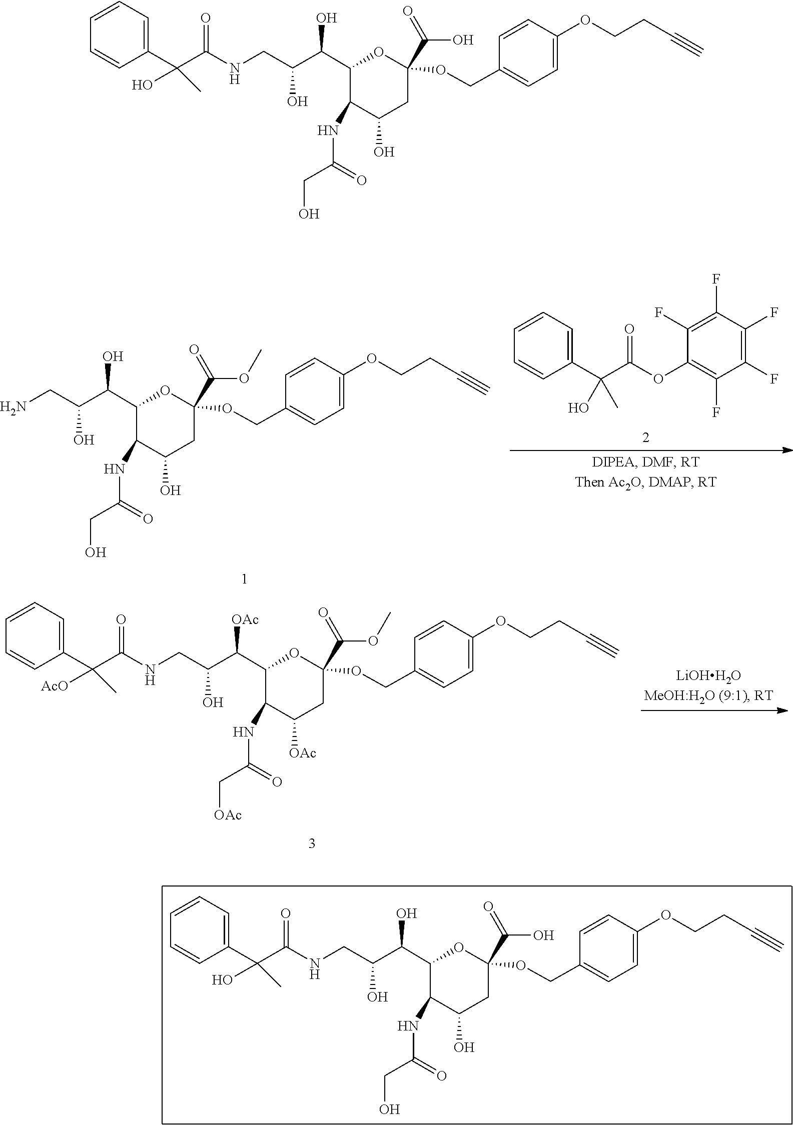

### Synthesis of (1R,2R)-3-(2-acetoxy-2-phenylpropanamido)-1-((2R,3R,4S,6R)-4-acetoxy-3-(2-acetoxyacetamido)-6-((4-(but-3-yn-1-yloxy)benzyl)oxy)-6-(methoxycarbonyl)tetrahydro-2H-pyran-2-yl)propane-1,2-diyl diacetate (3)

To a stirred solution of methyl (2R,4S,5R,6R)-6-((1R,2R)-3-amino-1,2-dihydroxypropyl)-2-((4-(but-3-yn-1-yloxy)benzyl)oxy)-4-hydroxy-5-(2-hydroxyacetamido)tetrahydro-2H-pyran-2-carboxylate (1, 0.40 g, 0.806 mmol) and perfluorophenyl 2-hydroxy-2-phenylpropanoate (2, 0.268 g, 0.806 mmol) in N, N-dimethylformamide (4.0 mL), was added DIPEA (0.702 mL, 4.03 mmol) at 0° C. and the reaction mixture was stirred at room temperature for 4 h. After completion acetic anhydride (0.914 mL, 8.06 mmol) and 4-dimethylaminopyridine (0.0098 g, 0.0806 mmol) were added to the reaction mixture which was stirred for 12 h. The reaction mixture was concentrated under reduced pressure and the residue was taken up in ethyl acetate, washed by saturated solution of sodium bicarbonate and dried over sodium sulfate. The organic layer was filtered, and filtrate was concentrated under reduced pressure. The crude was purified by flash column chromatography using ethyl acetate and heptane as eluent to afford the desired product. Yield: 0.30 g, 43%; LCMS m/z 853.0 [M−H]−.

### Synthesis of (2R,4S,5R,6R)-2-((4-(but-3-yn-1-yloxy)benzyl)oxy)-6-((1R,2R)-1,2-dihydroxy-3-(2-hydroxy-2-phenylpropanamido)propyl)-4-hydroxy-5-(2-hydroxyacetamido)tetrahydro-2H-pyran-2-carboxylic acid (Compound 169)

To a stirred solution of (1R,2R)-3-(2-acetoxy-2-phenylpropanamido)-1-((2R,3R,4S,6R)-4-acetoxy-3-(2-acetoxyacetamido)-6-((4-(but-3-yn-1-yloxy)benzyl)oxy)-6-(methoxycarbonyl)tetrahydro-2H-pyran-2-yl)propane-1,2-diyl diacetate (3, 0.30 g, 0.35 mmol) in methanol (3.0 mL), was added a solution of lithium hydroxide monohydrate (0.088 g, 2.11 mmol) in water (0.30 mL). The reaction mixture was stirred at room temperature for 6 h. After completion, the reaction mixture was treated with acidic resin (Dowex 50, H⁺) to reach pH ˜6 and the suspension was filtered. The filtrate was concentrated under reduced pressure. The crude residue obtained was purified by prep HPLC using water and acetonitrile (+0.1% formic acid) as eluent to afford the desired product. Yield: 0.021 g, 10%; LCMS m/z 631.40 [M+H]⁺; ¹H NMR (400 MHz, methanol-d₄) δ 7.58-7.57 (m, 2H), 7.32-7.15 (m, 5H), 6.89-6.87 (m, 2H), 4.75-4.68 (m, 1H), 4.40 (t, J=11.1 Hz, 1H), 4.07 (dt, J=6.8 & 1.2 Hz, 2H), 4.03-3.99 (m, 2H), 3.88-3.78 (m, 4H), 3.67-3.60 (m, 1H), 3.43-3.39 (m, 1H), 3.30-3.27 (m, 1H), 2.78-2.74 (m, 1H), 2.63 (dt, J=6.8 & 2.4 Hz, 2H), 2.32 (t, J=2.0 Hz, 1H), 1.80-1.73 (m, 4H).

### (2R,4S,5R,6R)-6-((1R,2R)-1,2-dihydroxy-3-(2-(4′-hydroxy-3-methyl-[1,1′-biphenyl]-4-yl)acetamido)propyl)-4-hydroxy-5-(2-hydroxyacetamido)-2-((4-(prop-2-yn-1-yloxy)benzyl)oxy)tetrahydro-2H-pyran-2-carboxylic acid (Compound 56)

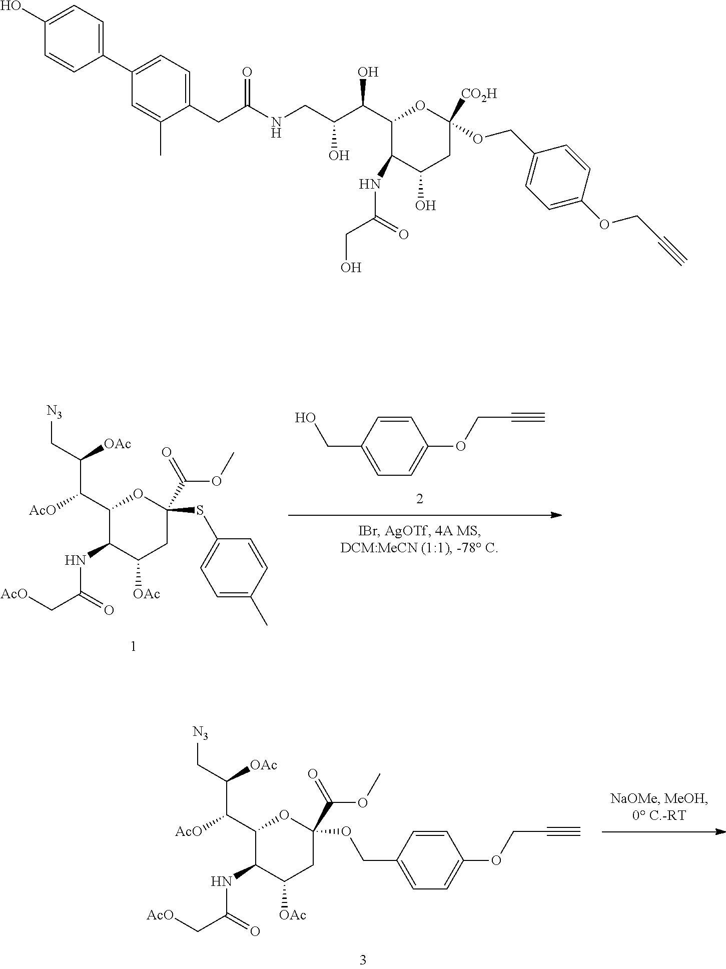 [SMILES: C#CCOc1ccc(CO[C@]2(C(=O)O)C[C@H](O)[C@@H](NC(=O)CO)[C@H]([C@H](O)[C@H](O)CNC(=O)Cc3ccc(-c4ccc(O)cc4)cc3C)O2)cc1 ; COC(=O)[C@]1(Sc2ccc(C)cc2)C[C@H](OC(C)=O)[C@@H](NC(=O)COC(C)=O)[C@H]([C@H](OC(C)=O)[C@@H](CN=[N+]=[N-])OC(C)=O)O1 ; C#CCOc1ccc(CO)cc1 ; C#CCOc1ccc(CO[C@]2(C(=O)OC)C[C@H](OC(C)=O)[C@@H](NC(=O)COC(C)=O)[C@H]([C@H](OC(C)=O)[C@@H](CN=[N+]=[N-])OC(C)=O)O2)cc1]

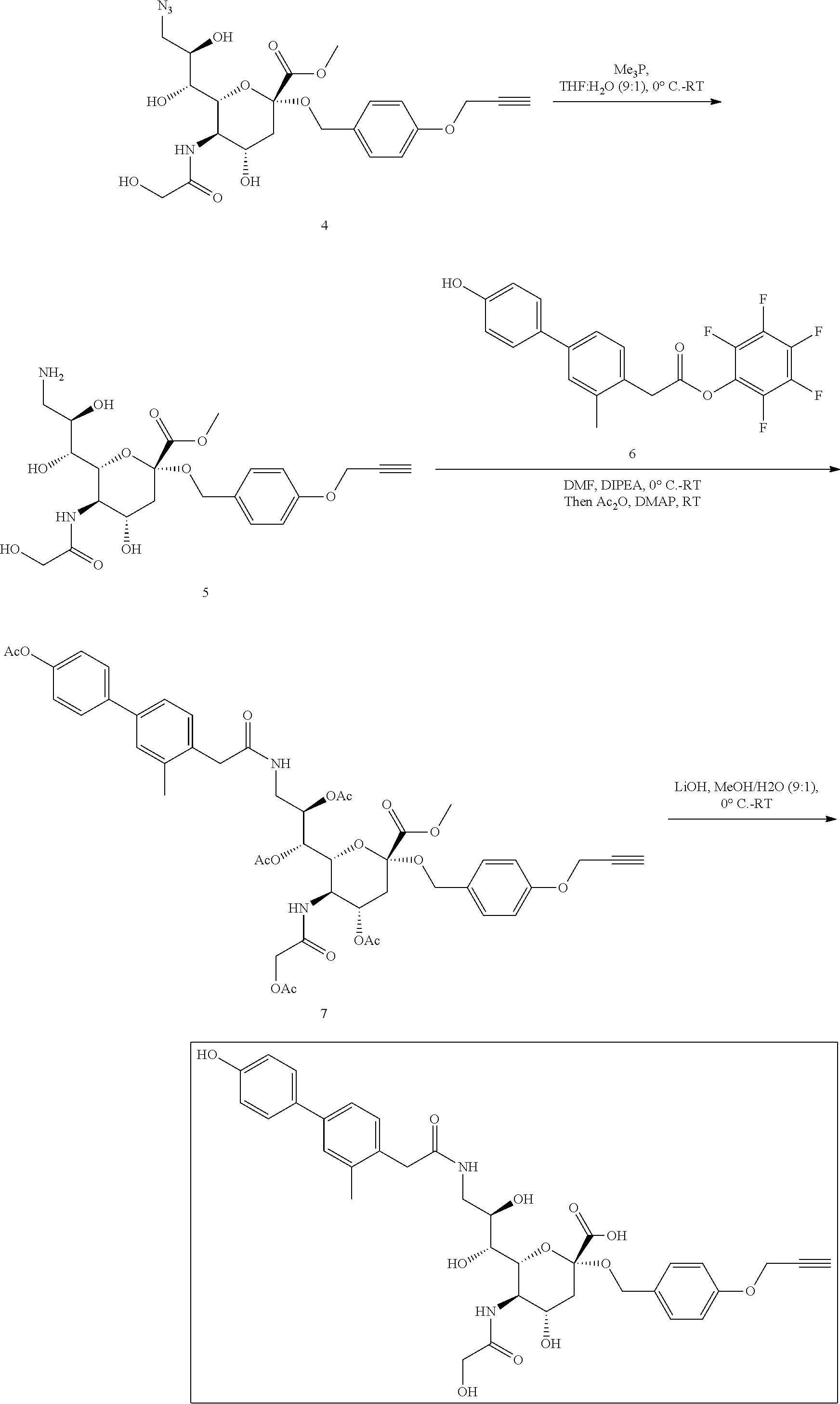 [SMILES: C#CCOc1ccc(CO[C@]2(C(=O)OC)C[C@H](O)[C@@H](NC(=O)CO)[C@H]([C@H](O)[C@H](O)CN=[N+]=[N-])O2)cc1 ; C#CCOc1ccc(CO[C@]2(C(=O)OC)C[C@H](O)[C@@H](NC(=O)CO)[C@H]([C@H](O)[C@H](O)CN)O2)cc1 ; Cc1cc(-c2ccc(O)cc2)ccc1CC(=O)Oc1c(F)c(F)c(F)c(F)c1F ; C#CCOc1ccc(CO[C@]2(C(=O)OC)C[C@H](OC(C)=O)[C@@H](NC(=O)COC(C)=O)[C@H]([C@H](OC(C)=O)[C@@H](CNC(=O)Cc3ccc(-c4ccc(OC(C)=O)cc4)cc3C)OC(C)=O)O2)cc1 ; C#CCOc1ccc(CO[C@]2(C(=O)O)C[C@H](O)[C@@H](NC(=O)CO)[C@H]([C@H](O)[C@H](O)CNC(=O)Cc3ccc(-c4ccc(O)cc4)cc3C)O2)cc1]

### Synthesis of (1R,2R)-1-((2R,3R,4S,6R)-4-acetoxy-3-(2-acetoxyacetamido)-6-(methoxycarbonyl)-6-((4-(prop-2-yn-1-yloxy)benzyl)oxy)tetrahydro-2H-pyran-2-yl)-3-azidopropane-1,2-diyl diacetate (3)

A solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-azidopropane-1,2-diyl diacetate (2.5 g, 10.3 mmol) and (4-(prop-2-yn-1-yloxy)phenyl)methanol (2, 3.17 g, 19.6 mmol), silver(I) trifluoromethanesulfonate (2.01 g, 7.83 mmol) and activated 4 Å powdered molecular sieves (1.00 g) in anhydrous dichloromethane (120 mL) and anhydrous acetonitrile (175 mL, 3.45 mol) was stirred at room temperature for 1 h under nitrogen atmosphere. The solution was cooled to −78° C. and iodine monobromide (1.21 g, 5.87 mmol) in dichloromethane (5.0 mL) was added dropwise to the reaction mixture and was stirred at the same temperature for 2 h. After completion the reaction mixture was quenched with triethylamine (2.0 mL) and warmed to room temperature. The reaction mixture was filtered, and the filtrate was washed with a saturated solution of sodium bicarbonate and dried over sodium sulfate, filtered and concentrated. The crude product was purified by column chromatography using ethyl acetate and heptane as eluent to afford the desired product. Yield: 2.0 g, 75%; LCMS m/z 694.35 [M+18]⁺.

### Synthesis of methyl (2R,4S,5R,6R)-6-((1R,2R)-3-azido-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylate (4)

(1R,2R)-1-((2R,3R,4S,6R)-4-acetoxy-3-(2-acetoxyacetamido)-6-(methoxycarbonyl)-6-((4-(prop-2-yn-1-yloxy)benzyl)oxy)tetrahydro-2H-pyran-2-yl)-3-azidopropane-1,2-diyl diacetate (3, 2.0 g, 2.96 mmol) was dissolved in methanol (60.0 mL) and 25% sodium methoxide in methanol (0.5 mL, 0.3 mmol, pH should not be more than 9) was added. The reaction mixture was stirred for 30 mins at room temperature. The solution was neutralized with Dowex-Hydrogen form to reach neutral pH, filtered and the filtrate was concentrated. The crude was purified by column chromatography using ethyl acetate and heptane as eluent to afford the desired product. Yield: 1.30 g, 66%; LCMS m/z 506.90 [M−H]⁻.

### Synthesis of methyl (2R,4S,5R,6R)-6-((1R,2R)-3-amino-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylate (5)

To a stirred solution of methyl (2R,4S,5R,6R)-6-((1R,2R)-3-azido-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylate (4, 1.0 g, 1.97 mmol) in tetrahydrofuran (8 mL) and water (2 mL), was added 1 M solution of trimethylphosphine (2.58 g, 9.83 mmol) in tetrahydrofuran at 0° C. and the reaction was stirred at room temperature for 12 h. After completion the mixture was concentrated, and the crude residue obtained was partitioned between ethyl acetate (50 ml) and water (100 ml). The aqueous layer was lyophilized to afford the desired product as a crude. Yield: 0.42 g, 44.26%; LCMS m/z 483.20 [M+H]+.

### Synthesis of (1R,2R)-1-((2R,3R,4S,6R)-4-acetoxy-3-(2-hydroxyacetamido)-6-(methoxycarbonyl)-6-((4-(prop-2-yn-1-yloxy)benzyl)oxy)tetrahydro-2H-pyran-2-yl)-3-(2-(4′-acetoxy-3-methyl-[1,1′-biphenyl]-4-yl)acetamido)propane-1,2-diyl diacetate (7)

To a solution of methyl (2R,4S,5R,6R)-6-((1R,2R)-3-amino-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylate (5, 0.1 g, 0.21 mmol) and 2,5-dioxopyrrolidin-1-yl 2-(4′-hydroxy-[1,1′-biphenyl]-4-yl)acetate (6, 0.068 g, 0.17 mmol) in N,N-dimethylformamide (5.0 mL), was added DIPEA (0.18 mL, 1.04 mmol) at 0° C. and the reaction mixture was stirred at room temperature for 4 h. After completion acetic anhydride (2.0 ml) and 4-dimethylaminopyridine (0.013 g, 0.10 mmol) were added to the reaction mixture which was stirred for 12 h. After completion the mixture was concentrated and the residue was taken up in ethyl acetate, washed with a saturated solution of sodium bicarbonate, dried over sodium sulfate, filtered and concentrated. The crude residue obtained was purified by flash column chromatography using ethyl acetate and heptane as eluent to afford the desired product. Yield: 0.1 g, 55%; LCMS m/z 917.00 [M+H]+.

### Synthesis of (2R,4S,5R,6R)-6-((1R,2R)-1,2-dihydroxy-3-(2-(4′-hydroxy-3-methyl-[1,1′-biphenyl]-4-yl)acetamido)propyl)-4-hydroxy-5-(2-hydroxyacetamido)-2-((4-(prop-2-yn-1-yloxy)benzyl)oxy)tetrahydro-2H-pyran-2-carboxylic acid (Compound 56)

To a stirred solution of (1R,2R)-1-((2R,3R,4S,6R)-4-acetoxy-3-(2-hydroxyacetamido)-6-(methoxycarbonyl)-6-((4-(prop-2-yn-1-yloxy)benzyl)oxy)tetrahydro-2H-pyran-2-yl)-3-(2-(4′-acetoxy-3-methyl-[1,1′-biphenyl]-4-yl)acetamido)propane-1,2-diyl diacetate (7, 0.10 g, 0.11 mmol) in methanol (5 mL), was added a solution of lithium hydroxide monohydrate (0.026 g, 0.65 mmol) in water (1.0 mL). The reaction mixture was stirred at room temperature for 6 h. After completion, the reaction mixture was treated with acidic resin (Dowex 50, H⁺) to reach pH ˜6 and the suspension was filtered. The filtrate was concentrated, and the crude residue obtained was purified by prep HPLC using water and acetonitrile (+0.1% FA) as eluent to afford the desired product. Yield: 0.005 g, 6%; LCMS m/z 693.40 [M+H]⁺; ¹H NMR (400 MHz, methanol-d₄) δ 7.39 (d, J=8.4 Hz, 2H), 7.33-7.29 (m, 2H), 7.26-7.22 (m, 3H), 6.91 (d, J=8.4 Hz, 2H), 6.82 (d, J=8.8 Hz, 2H), 4.73 (d, J=10.8 Hz, 1H), 4.69 (d, J=2.4 Hz, 2H), 4.40 (d, J=11.2 Hz, 1H), 4.12 (s, 2H), 4.02-3.60 (m, 4H), 3.54 (s, 2H), 3.39 (d, J=8.8 Hz, 1H), 3.31-3.24 (m, 1H), 2.92 (t, J=2.4 Hz, 1H), 2.83-2.78 (m, 1H), 2.33 (s, 3H), 1.76 (m, 1H).

### (2R,4S,5R,6R)-6-((1R,2R)-3-(2-(3-chloro-4′-hydroxy-[1,1′-biphenyl]-4-yl)acetamido)-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylic acid (Compound 52)

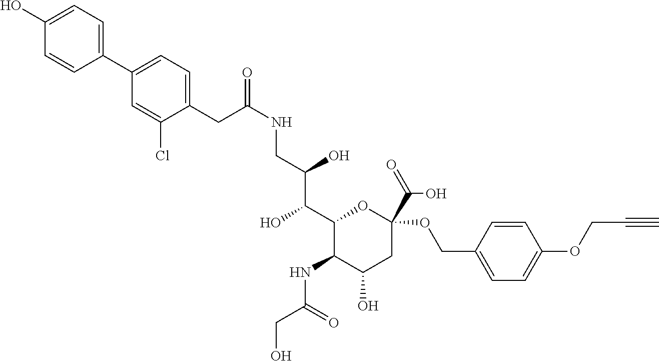 [SMILES: C#CCOc1ccc(CO[C@]2(C(=O)O)C[C@H](O)[C@@H](NC(=O)CO)[C@H]([C@H](O)[C@H](O)CNC(=O)Cc3ccc(-c4ccc(O)cc4)cc3Cl)O2)cc1]

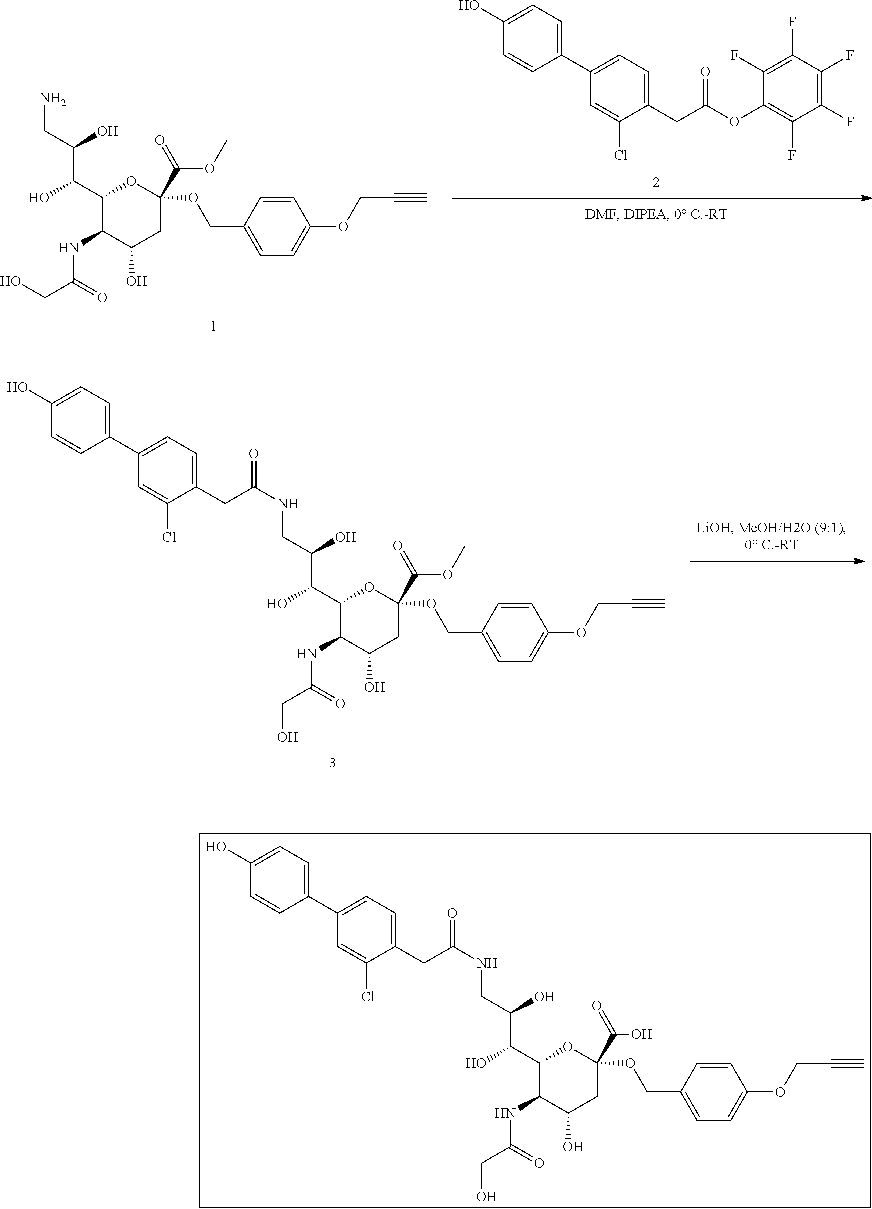 [SMILES: C#CCOc1ccc(CO[C@]2(C(=O)OC)C[C@H](O)[C@@H](NC(=O)CO)[C@H]([C@H](O)[C@H](O)CN)O2)cc1 ; O=C(Cc1ccc(-c2ccc(O)cc2)cc1Cl)Oc1c(F)c(F)c(F)c(F)c1F ; C#CCOc1ccc(CO[C@]2(C(=O)OC)C[C@H](O)[C@@H](NC(=O)CO)[C@H]([C@H](O)[C@H](O)CNC(=O)Cc3ccc(-c4ccc(O)cc4)cc3Cl)O2)cc1 ; C#CCOc1ccc(CO[C@]2(C(=O)O)C[C@H](O)[C@@H](NC(=O)CO)[C@H]([C@H](O)[C@H](O)CNC(=O)Cc3ccc(-c4ccc(O)cc4)cc3Cl)O2)cc1]

### Synthesis of methyl (2R,4S,5R,6R)-6-((1R,2R)-3-(2-(3-chloro-4′-hydroxy-[1,1′-biphenyl]-4-yl)acetamido)-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylate (3)

To a solution of methyl (2R,4S,5R,6R)-6-((1R,2R)-3-amino-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylate (0.09 g, 0.19 mmol) and perfluorophenyl 2-(3-chloro-4′-hydroxy-[1,1′-biphenyl]-4-yl)acetate (2, 0.064 g, 0.15 mmol) in N,N-dimethylformamide (2.0 mL), was added DIPEA (0.002 mL, 0.93 mmol) at 0° C. and the reaction mixture was stirred at room temperature for 4 h. The crude residue obtained was purified by flash column chromatography using dichloromethane and methanol as eluent to afford the desired product. Yield: 0.06 g, 44%; LCMS m/z 726.80 [M+H]+.

### Synthesis of (2R,4S,5R,6R)-6-((1R,2R)-3-(2-(3-chloro-4′-hydroxy-[1,1′-biphenyl]-4-yl)acetamido)-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylic acid (Compound 52)

To a stirred solution of methyl (2R,4S,5R,6R)-6-((1R,2R)-3-(2-(3-chloro-4′-hydroxy-[1,1′-biphenyl]-4-yl)acetamido)-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylate (3, 0.06 g, 0.18 mmol) in methanol (5 mL), was added a solution of lithium hydroxide monohydrate (0.007 g, 0.18 mmol) in water (1.0 mL). The reaction mixture was stirred at room temperature for 6 h. After completion, the reaction mixture was treated with acidic resin (Dowex 50, H⁺) to reach pH ˜6 and the suspension was filtered. The filtrate was concentrated, and the crude residue obtained was purified by prep HPLC using water and acetonitrile (+0.1% TFA) as eluent to afford the desired product. Yield: 0.012 g, 20%; LCMS m/z 713.35 [M+H]⁺; ¹H NMR (400 MHz, methanol-d₄) δ 7.93 (d, J=7.2 Hz. 1H), 7.85 (m, 1H), 7.54 (d, J=1.6 Hz, 1H), 7.44-7.36 (m, 4H), 7.26 (d, J=8.4 Hz, 2H), 6.92 (d, J=8.8 Hz, 2H), 6.84 (d, J=8.4 Hz, 2H), 4.75 (d, J=10.8 Hz, 1H), 4.69 (d, J=2.4 Hz, 2H), 4.42 (d, J=10.8 Hz, 1H), 4.04 (s, 2H), 3.98-3.69 (m, 7H), 3.42 (d, J=8.8 Hz, 1H), 3.30-3.27 (m, 1H), 2.92 (t, J=2.4 Hz, 1H), 2.77 (dd, J=12.4 & 4.0 Hz, 1H), 1.76 (t, J=12.0 Hz, 1H).

### (2R,4S,5R,6R)-6-((1R,2R)-1,2-dihydroxy-3-(2-(4′-hydroxy-3-methoxy-[1,1′-biphenyl]-4-yl)acetamido)propyl)-4-hydroxy-5-(2-hydroxyacetamido)-2-((4-(prop-2-yn-1-yloxy)benzyl)oxy)tetrahydro-2H-pyran-2-carboxylic acid (Compound 51)

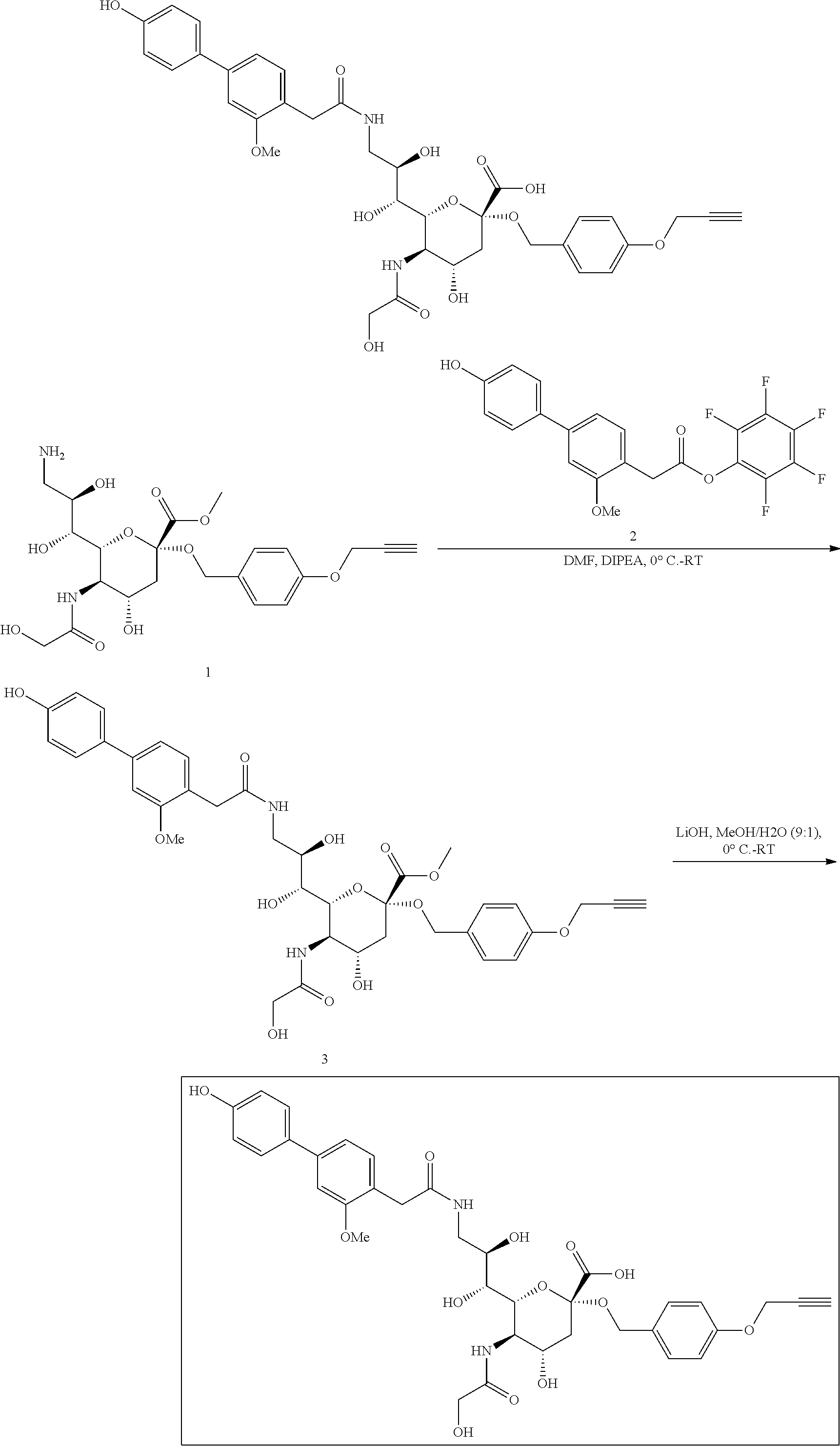 [SMILES: C#CCOc1ccc(CO[C@]2(C(=O)O)C[C@H](O)[C@@H](NC(=O)CO)[C@H]([C@H](O)[C@H](O)CNC(=O)Cc3ccc(-c4ccc(O)cc4)cc3OC)O2)cc1 ; C#CCOc1ccc(CO[C@]2(C(=O)OC)C[C@H](O)[C@@H](NC(=O)CO)[C@H]([C@H](O)[C@H](O)CN)O2)cc1 ; COc1cc(-c2ccc(O)cc2)ccc1CC(=O)Oc1c(F)c(F)c(F)c(F)c1F ; C#CCOc1ccc(CO[C@]2(C(=O)OC)C[C@H](O)[C@@H](NC(=O)CO)[C@H]([C@H](O)[C@H](O)CNC(=O)Cc3ccc(-c4ccc(O)cc4)cc3OC)O2)cc1 ; C#CCOc1ccc(CO[C@]2(C(=O)O)C[C@H](O)[C@@H](NC(=O)CO)[C@H]([C@H](O)[C@H](O)CNC(=O)Cc3ccc(-c4ccc(O)cc4)cc3OC)O2)cc1]

### Synthesis of methyl (2R,4S,5R,6R)-6-((1R,2R)-1,2-dihydroxy-3-(2-(4′-hydroxy-3-methoxy-[1,1′-biphenyl]-4-yl)acetamido)propyl)-4-hydroxy-5-(2-hydroxyacetamido)-2-((4-(prop-2-yn-1-yloxy)benzyl)oxy)tetrahydro-2H-pyran-2-carboxylate (3)

To a solution of methyl (2R,4S,5R,6R)-6-((1R,2R)-3-amino-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylate (0.20 g, 0.42 mmol) and perfluorophenyl 2-(4′-hydroxy-3-methoxy-[1,1′-biphenyl]-4-yl)acetate (2, 0.17 g, 0.42 mmol) in N,N-dimethylformamide (5.0 mL), was added DIPEA (0.003 mL, 2.07 mmol) at 0° C. and the reaction mixture was stirred at room temperature for 4 h. The crude residue obtained was purified by flash column chromatography using dichloromethane and methanol as eluent to afford the desired product. Yield: 0.15 g, 50%; LCMS m/z 722.9 [M+H]+.

### Synthesis of (2R,4S,5R,6R)-6-((1R,2R)-1,2-dihydroxy-3-(2-(4′-hydroxy-3-methoxy-[1,1′-biphenyl]-4-yl)acetamido)propyl)-4-hydroxy-5-(2-hydroxyacetamido)-2-((4-(prop-2-yn-1-yloxy)benzyl)oxy)tetrahydro-2H-pyran-2-carboxylic acid (Compound 51)

To a stirred solution of methyl (2R,4S,5R,6R)-6-((1R,2R)-1,2-dihydroxy-3-(2-(4′-hydroxy-3-methoxy-[1,1′-biphenyl]-4-yl)acetamido)propyl)-4-hydroxy-5-(2-hydroxyacetamido)-2-((4-(prop-2-yn-1-yloxy)benzyl)oxy)tetrahydro-2H-pyran-2-carboxylate (3, 0.15 g, 0.21 mmol) in methanol (5 mL), was added a solution of lithium hydroxide monohydrate (0.008 g, 0.21 mmol) in water (1.0 mL). The reaction mixture was stirred at room temperature for 6 h. The reaction mixture was treated with acidic resin (Dowex 50, H⁺) to reach pH˜6 and the suspension was filtered. The filtrate was concentrated, and the crude residue obtained was purified by prep HPLC using water and acetonitrile (+0.1% TFA) as eluent to afford the desired product. Yield: 0.036 g, 25%; LCMS m/z 709.4 [M+H]⁺; ¹H NMR (400 MHz, methanol-d₄) δ 7.91 (d, J=7.6 Hz, 1H), 7.42 (d, J=8.4 Hz, 2H), 7.24-7.21 (m, 3H), 7.07-7.06 (m, 2H), 6.90 (d, J=8.8 Hz, 2H), 6.83 (d, J=8.8 Hz, 2H), 4.73-4.70 (m, 1H), 4.69 (d, J=2.0 Hz, 2H), 4.39 (d, J=11.2 Hz, 1H), 4.04 (s, 2H), 4.01-3.73 (m, 7H), 3.69 (dd, J=13.6 & 3.2 Hz, 1H), 3.56 (d, J=2.8 Hz, 2H), 3.39 (d, J=8.4 Hz, 1H), 3.31-3.24 (m, 1H), 2.92 (t, J=2.4 Hz, 1H), 2.75 (dd, J=12.4 & 4.0 Hz, 1H), 1.78 (t, J=12.0 Hz, 1H).

### (2R,4S,5R,6R)-6-((1R,2R)-3-(2-(2-chloro-4′-hydroxy-[1,1′-biphenyl]-4-yl)acetamido)-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylic acid (Compound 47)

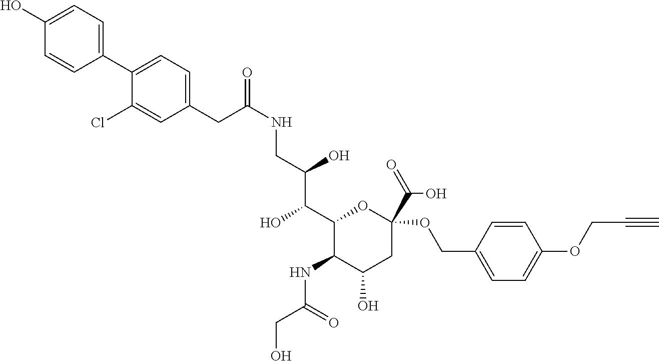 [SMILES: C#CCOc1ccc(CO[C@]2(C(=O)O)C[C@H](O)[C@@H](NC(=O)CO)[C@H]([C@H](O)[C@H](O)CNC(=O)Cc3ccc(-c4ccc(O)cc4)c(Cl)c3)O2)cc1]

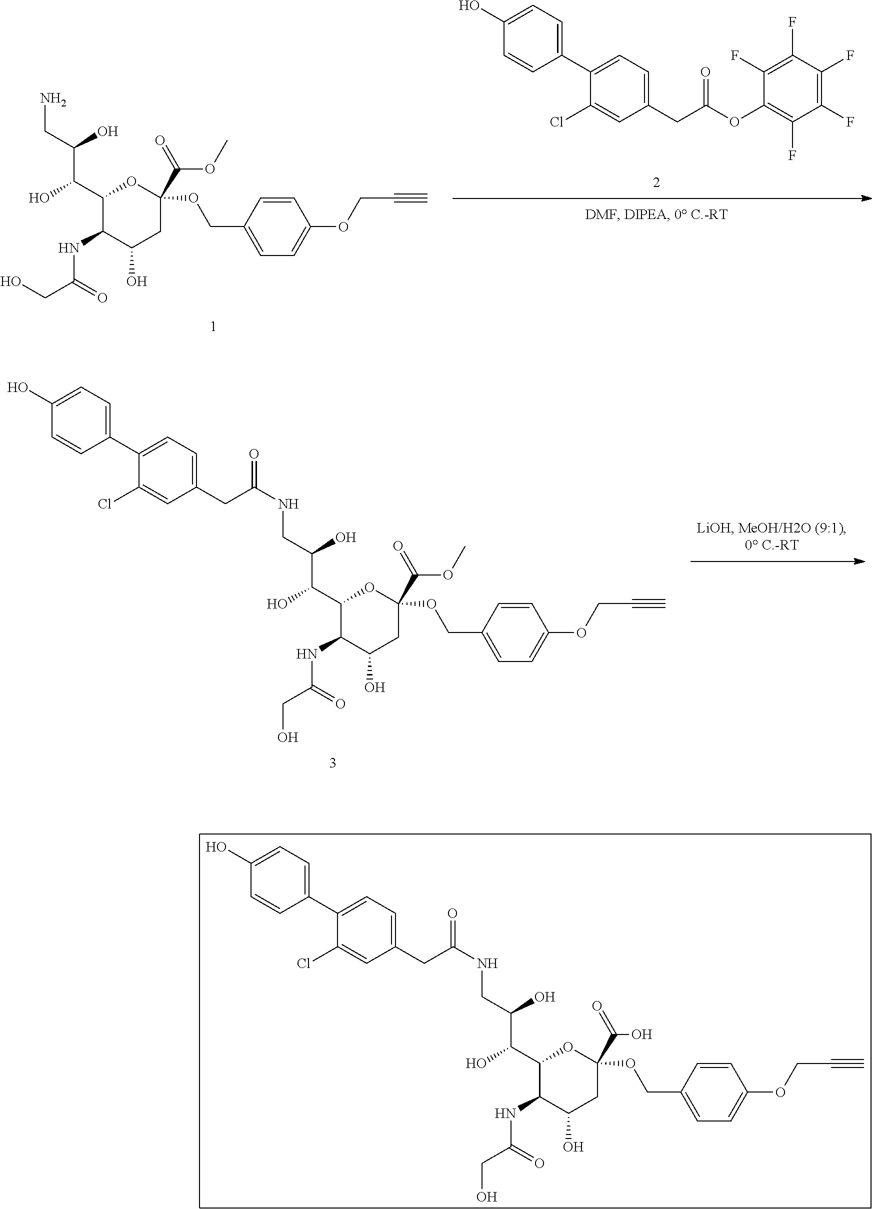 [SMILES: C#CCOc1ccc(CO[C@]2(C(=O)OC)C[C@H](O)[C@@H](NC(=O)CO)[C@H]([C@H](O)[C@H](O)CN)O2)cc1 ; O=C(Cc1ccc(-c2ccc(O)cc2)c(Cl)c1)Oc1c(F)c(F)c(F)c(F)c1F ; C#CCOc1ccc(CO[C@]2(C(=O)OC)C[C@H](O)[C@@H](NC(=O)CO)[C@H]([C@H](O)[C@H](O)CNC(=O)Cc3ccc(-c4ccc(O)cc4)c(Cl)c3)O2)cc1 ; C#CCOc1ccc(CO[C@]2(C(=O)O)C[C@H](O)[C@@H](NC(=O)CO)[C@H]([C@H](O)[C@H](O)CNC(=O)Cc3ccc(-c4ccc(O)cc4)c(Cl)c3)O2)cc1]

### Synthesis of methyl (2R,4S,5R,6R)-6-((1R,2R)-3-(2-(2-chloro-4′-hydroxy-[1,1′-biphenyl]-4-yl)acetamido)-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylate (3)

To a solution of methyl (2R,4S,5R,6R)-6-((1R,2R)-3-amino-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylate (0.20 g, 0.42 mmol) and perfluorophenyl 2-(2-chloro-4′-hydroxy-[1,1′-biphenyl]-4-yl)acetate (2, 0.18 g, 0.42 mmol) in N,N-dimethylformamide (5.0 mL), was added DIPEA (0.003 mL, 2.07 mmol) at 0° C. and the reaction mixture was stirred at room temperature for 4 h. The crude residue obtained was purified by flash column chromatography using dichloromethane and methanol as eluent to afford the desired product. Yield: 0.126 g, 42%; LCMS m/z 726.9 [M+H]+.

### Synthesis of (2R,4S,5R,6R)-6-((1R,2R)-3-(2-(2-chloro-4′-hydroxy-[1,1′-biphenyl]-4-yl)acetamido)-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylic acid

To a stirred solution of methyl (2R,4S,5R,6R)-6-((1R,2R)-3-(2-(2-chloro-4′-hydroxy-[1,1′-biphenyl]-4-yl)acetamido)-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylate (3, 0.12 g, 0.16 mmol) in methanol (5 mL), was added a solution of lithium hydroxide monohydrate (0.004 g, 0.16 mmol) in water (1.0 mL). The reaction mixture was stirred at room temperature for 6 h. The reaction mixture was treated with acidic resin (Dowex 50, H⁺) to reach pH ˜6 and the suspension was filtered. The filtrate was concentrated, and the crude residue obtained was purified by prep HPLC using water and acetonitrile (+0.1% TFA) as eluent to afford the desired product. Yield: 0.021 g, 25%; LCMS m/z 713.40 [M+H]⁺; ¹H NMR (400 MHz, methanol-d₄) δ 7.93 (d, J=7.6 Hz, 1H), 7.43 (s, 1H), 7.26-7.21 (m, 4H), 7.19 (d, J=8.8 Hz, 2H), 6.92 (d, J=8.4 Hz, 2H), 6.81 (d, J=8.4 Hz, 2H), 4.74 (d, J=10.8 Hz, 1H), 4.69 (d, J=2.0 Hz, 2H), 4.41 (d, J=10.8 Hz, 1H), 4.03 (s, 2H), 3.97-3.73 (m, 4H), 3.70 (dd, J=13.6 & 2.8 Hz, 1H), 3.56 (s, 2H), 3.42 (d, J=8.4 Hz, 1H), 3.31-3.23 (m, 1H), 2.92 (t, J=2.4 Hz, 1H), 2.76 (dd, J=12.4 & 4.0 Hz, 1H), 1.81-1.75 (m, 1H).

### (2R,4S,5R,6R)-6-((1R,2R)-1,2-dihydroxy-3-(2-(3-hydroxyphenyl)acetamido)propyl)-4-hydroxy-5-(2-hydroxyacetamido)-2-((4-(prop-2-yn-1-yloxy)benzyl)oxy)tetrahydro-2H-pyran-2-carboxylic acid (Compound 150)

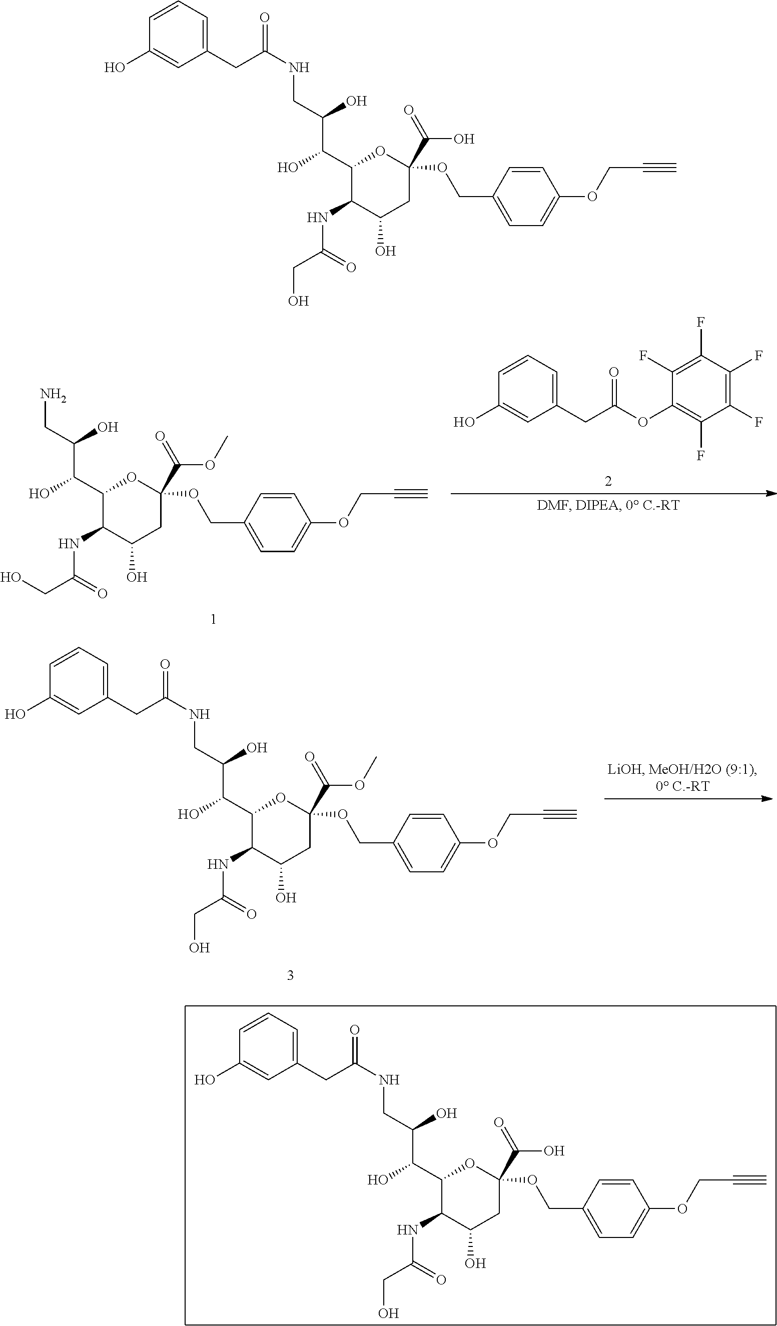 [SMILES: C#CCOc1ccc(CO[C@]2(C(=O)O)C[C@H](O)[C@@H](NC(=O)CO)[C@H]([C@H](O)[C@H](O)CNC(=O)Cc3cccc(O)c3)O2)cc1 ; C#CCOc1ccc(CO[C@]2(C(=O)OC)C[C@H](O)[C@@H](NC(=O)CO)[C@H]([C@H](O)[C@H](O)CN)O2)cc1 ; O=C(Cc1cccc(O)c1)Oc1c(F)c(F)c(F)c(F)c1F ; C#CCOc1ccc(CO[C@]2(C(=O)OC)C[C@H](O)[C@@H](NC(=O)CO)[C@H]([C@H](O)[C@H](O)CNC(=O)Cc3cccc(O)c3)O2)cc1 ; C#CCOc1ccc(CO[C@]2(C(=O)O)C[C@H](O)[C@@H](NC(=O)CO)[C@H]([C@H](O)[C@H](O)CNC(=O)Cc3cccc(O)c3)O2)cc1]

### Synthesis of methyl (2R,4S,5R,6R)-6-((1R,2R)-1,2-dihydroxy-3-(2-(3-hydroxyphenyl)acetamido)propyl)-4-hydroxy-5-(2-hydroxyacetamido)-2-((4-(prop-2-yn-1-yloxy)benzyl)oxy)tetrahydro-2H-pyran-2-carboxylate (3)

To a solution of methyl (2R,4S,5R,6R)-6-((1R,2R)-3-amino-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylate (0.30 g, 0.62 mmol) and perfluorophenyl 2-(3-hydroxyphenyl)acetate (2, 0.19 g, 0.62 mmol) in N,N-dimethylformamide (5.0 mL), was added DIPEA (0.005 mL, 3.11 mmol) at 0° C. and the reaction mixture was stirred at room temperature for 4 h. The crude residue obtained was purified by flash column chromatography using dichloromethane and methanol as eluent to afford the desired product. Yield: 0.28 g, 75%; LCMS m/z 617.0 [M+H]+.

### Synthesis of (2R,4S,5R,6R)-6-((1R,2R)-1,2-dihydroxy-3-(2-(3-hydroxyphenyl)acetamido)propyl)-4-hydroxy-5-(2-hydroxyacetamido)-2-((4-(prop-2-yn-1-yloxy)benzyl)oxy)tetrahydro-2H-pyran-2-carboxylic acid (Compound 150)

To a stirred solution of methyl (2R,4S,5R,6R)-6-((1R,2R)-1,2-dihydroxy-3-(2-(3-hydroxyphenyl)acetamido)propyl)-4-hydroxy-5-(2-hydroxyacetamido)-2-((4-(prop-2-yn-1-yloxy)benzyl)oxy)tetrahydro-2H-pyran-2-carboxylate (3, 0.13 g, 0.21 mmol) in methanol (5 mL), was added a solution of lithium hydroxide monohydrate (0.009 g, 0.21 mmol) in water (1.0 mL). The reaction mixture was stirred at room temperature for 6 h. The reaction mixture was treated with acidic resin (Dowex 50, H⁺) to reach pH ˜6 and the suspension was filtered. The filtrate was concentrated, and the crude residue obtained was purified by prep HPLC using water and acetonitrile (+0.1% TFA) as eluent to afford the desired product. Yield: 0.04 g, 31%; LCMS m/z 603.50 [M+H]⁺; ¹H NMR (400 MHz, methanol-d₄) δ 7.26 (d, J=8.4 Hz, 2H), 7.09 (t, J=8.0 Hz, 1H), 6.96 (d, J=11.6 Hz, 2H), 6.75 (d, J=7.6 Hz, 2H), 6.65-6.63 (m, 1H), 4.74 (d, J=11.2 Hz, 1H), 4.71 (d, J=2.4 Hz, 2H), 4.43 (d, J=10.8 Hz, 1H), 4.03 (s, 2H), 3.95-3.77 (m, 4H), 3.66 (dd, J=13.8 & 3.0 Hz, 1H), 3.47 (s, 2H), 3.40 (dd, J=8.8 Hz & 1.6 Hz, 1H), 3.27-3.24 (m, 1H), 2.92 (t, J=2.4 Hz, 1H), 2.76 (dd, J=12.4 & 4.0 Hz, 1H), 1.78 (t, J=12.2 Hz, 1H).

### (2R,4S,5R,6R)-6-((1R,2R)-1,2-dihydroxy-3-(2-(4-hydroxyphenyl)acetamido)propyl)-4-hydroxy-5-(2-hydroxyacetamido)-2-((4-(prop-2-yn-1-yloxy)benzyl)oxy)tetrahydro-2H-pyran-2-carboxylic acid (Compound 146)

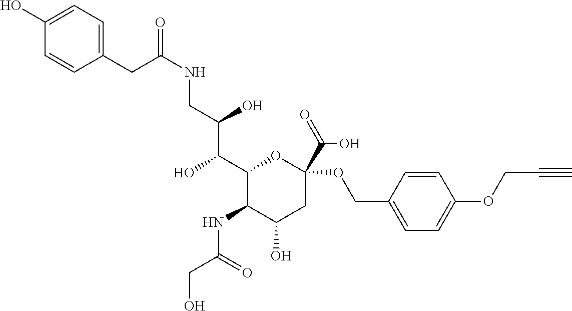 [SMILES: C#CCOc1ccc(CO[C@]2(C(=O)O)C[C@H](O)[C@@H](NC(=O)CO)[C@H]([C@H](O)[C@H](O)CNC(=O)Cc3ccc(O)cc3)O2)cc1]

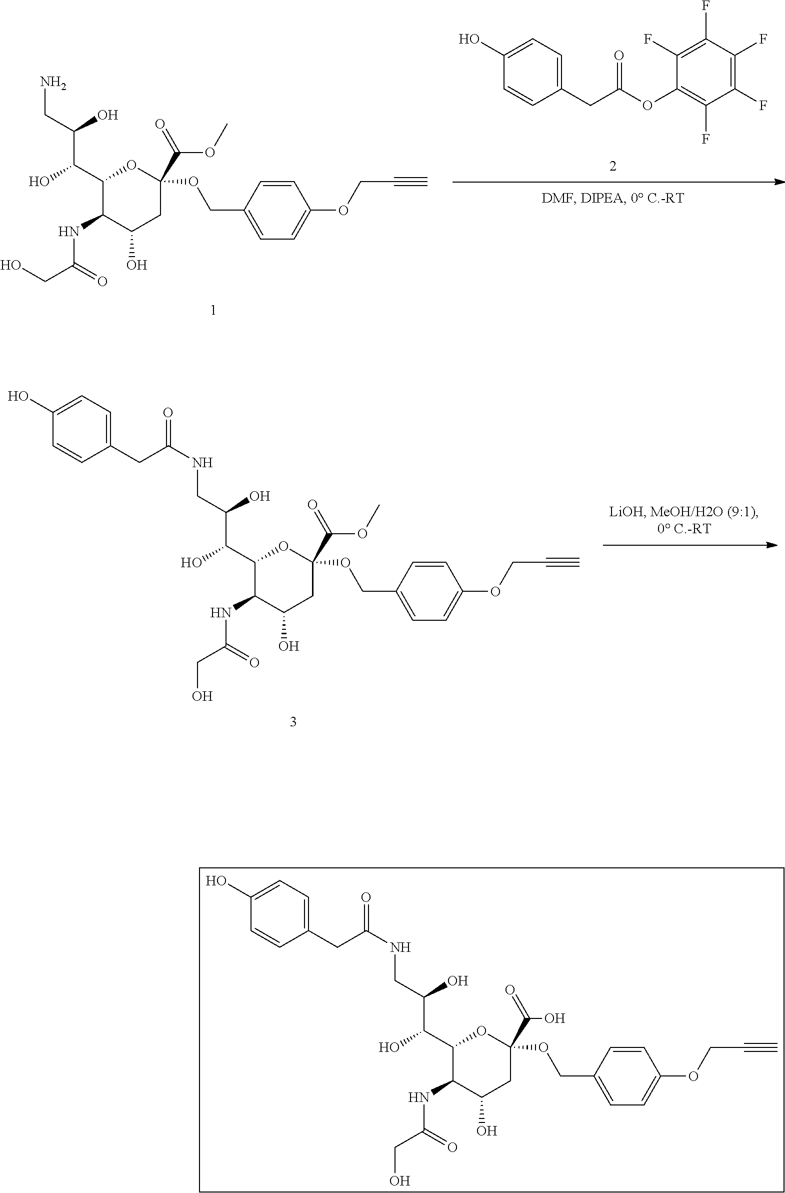 [SMILES: C#CCOc1ccc(CO[C@]2(C(=O)OC)C[C@H](O)[C@@H](NC(=O)CO)[C@H]([C@H](O)[C@H](O)CN)O2)cc1 ; O=C(Cc1ccc(O)cc1)Oc1c(F)c(F)c(F)c(F)c1F ; C#CCOc1ccc(CO[C@]2(C(=O)OC)C[C@H](O)[C@@H](NC(=O)CO)[C@H]([C@H](O)[C@H](O)CNC(=O)Cc3ccc(O)cc3)O2)cc1 ; C#CCOc1ccc(CO[C@]2(C(=O)O)C[C@H](O)[C@@H](NC(=O)CO)[C@H]([C@H](O)[C@H](O)CNC(=O)Cc3ccc(O)cc3)O2)cc1]

### Synthesis of methyl (2R,4S,5R,6R)-6-((1R,2R)-1,2-dihydroxy-3-(2-(4-hydroxyphenyl)acetamido)propyl)-4-hydroxy-5-(2-hydroxyacetamido)-2-((4-(prop-2-yn-1-yloxy)benzyl)oxy)tetrahydro-2H-pyran-2-carboxylate (3)

To a solution of methyl (2R,4S,5R,6R)-6-((1R,2R)-3-amino-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylate (0.15 g, 0.31 mmol) and perfluorophenyl 2-(4-hydroxyphenyl)acetate (2, 0.12 g, 0.37 mmol) in N,N-dimethylformamide (5.0 mL), was added DIPEA (0.2 mL, 1.55 mmol) at 0° C. and the reaction mixture was stirred at room temperature for 4 h. The crude residue obtained was purified by flash column chromatography using dichloromethane and methanol as eluent to afford the desired product. Yield: 0.15 g, 78%; LCMS m/z 617.0 [M+H]+.

### Synthesis of (2R,4S,5R,6R)-6-((1R,2R)-1,2-dihydroxy-3-(2-(4-hydroxyphenyl)acetamido)propyl)-4-hydroxy-5-(2-hydroxyacetamido)-2-((4-(prop-2-yn-1-yloxy)benzyl)oxy)tetrahydro-2H-pyran-2-carboxylic acid (Compound 146)

To a stirred solution of methyl (2R,4S,5R,6R)-6-((1R,2R)-1,2-dihydroxy-3-(2-(4-hydroxyphenyl)acetamido)propyl)-4-hydroxy-5-(2-hydroxyacetamido)-2-((4-(prop-2-yn-1-yloxy)benzyl)oxy)tetrahydro-2H-pyran-2-carboxylate (3, 0.15 g, 0.24 mmol) in methanol (5 mL), was added a solution of lithium hydroxide monohydrate (0.005 g, 0.24 mmol) in water (1.0 mL). The reaction mixture was stirred at room temperature for 6 h. The reaction mixture was treated with acidic resin (Dowex 50, H⁺) to reach pH ˜6 and the suspension was filtered. The filtrate was concentrated, and the crude residue obtained was purified by prep HPLC using water and acetonitrile (+0.1% TFA) as eluent to afford the desired product. Yield: 0.035 g, 24%; LCMS m/z 603.45 [M+H]⁺; ¹H NMR (400 MHz, methanol-d₄) δ ¹H NMR (400 MHz, Methanol-d₄) δ 7.92 (d, J=7.2 Hz, 1H), 7.26 (d, J=8.8 Hz, 2H), 7.10 (d, J=8.4 Hz, 2H), 6.94 (d, J=8.8 Hz, 2H), 6.71 (d, J=8.4 Hz, 2H), 4.74 (d, J=11.2 Hz, 1H), 4.71 (d, J=2.4 Hz, 2H), 4.42 (d, J=10.8 Hz, 1H), 4.03 (s, 2H), 3.94-3.76 (m, 4H), 3.64 (dd, J=13.6 & 2.8 Hz, 1H), 3.43 (s, 2H), 3.39 (d, J=8.8 Hz, 1H), 3.31-3.23 (m, 1H), 2.93 (t, J=2.4 Hz, 1H), 2.75 (dd, J=12.4 & 4.0 Hz, 1H), 1.78 (t, J=12.0 Hz, 1H).

### (2R,4S,5R,6R)-6-((1R,2R)-1,2-dihydroxy-3-(2-(2-hydroxyphenyl)acetamido)propyl)-4-hydroxy-5-(2-hydroxyacetamido)-2-((4-(prop-2-yn-1-yloxy)benzyl)oxy)tetrahydro-2H-pyran-2-carboxylic acid (Compound 145)

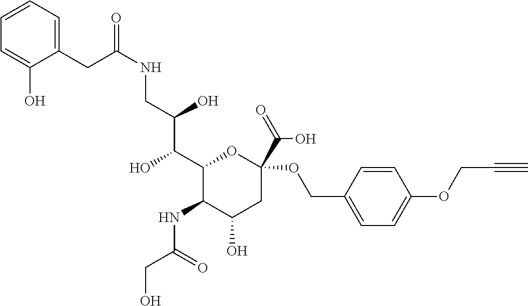 [SMILES: C#CCOc1ccc(CO[C@]2(C(=O)O)C[C@H](O)[C@@H](NC(=O)CO)[C@H]([C@H](O)[C@H](O)CNC(=O)Cc3ccccc3O)O2)cc1]

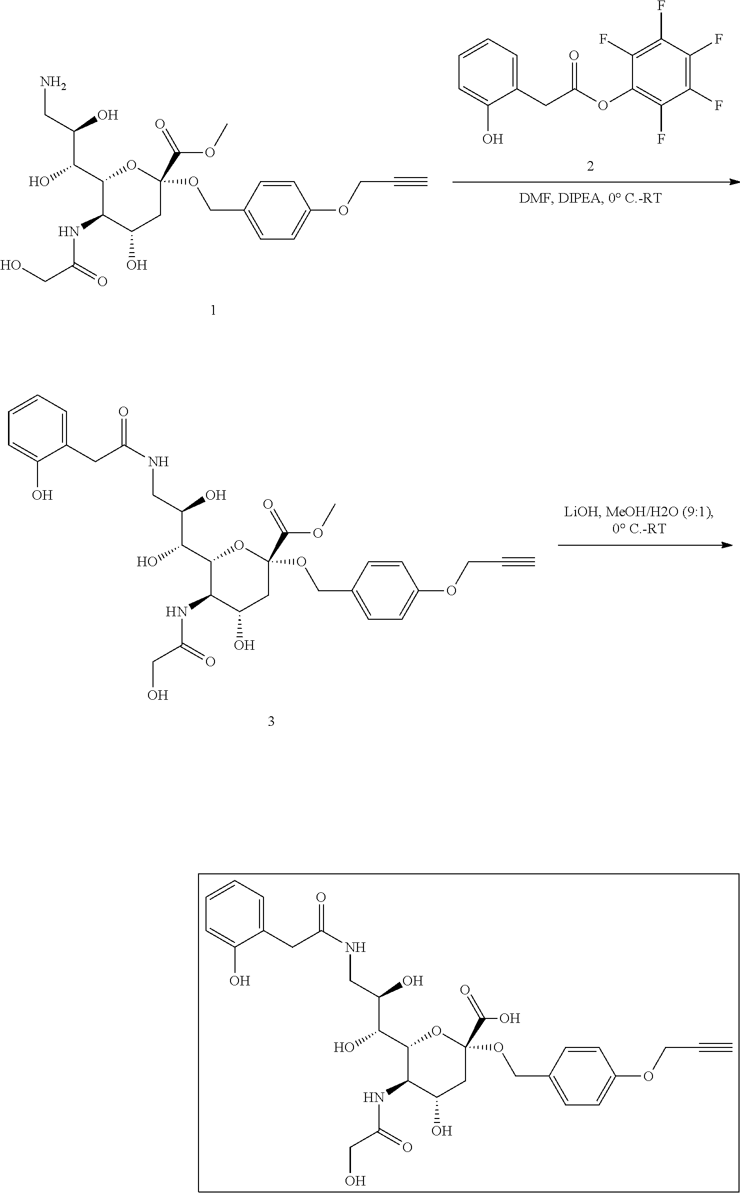 [SMILES: C#CCOc1ccc(CO[C@]2(C(=O)OC)C[C@H](O)[C@@H](NC(=O)CO)[C@H]([C@H](O)[C@H](O)CN)O2)cc1 ; O=C(Cc1ccccc1O)Oc1c(F)c(F)c(F)c(F)c1F ; C#CCOc1ccc(CO[C@]2(C(=O)OC)C[C@H](O)[C@@H](NC(=O)CO)[C@H]([C@H](O)[C@H](O)CNC(=O)Cc3ccccc3O)O2)cc1 ; C#CCOc1ccc(CO[C@]2(C(=O)O)C[C@H](O)[C@@H](NC(=O)CO)[C@H]([C@H](O)[C@H](O)CNC(=O)Cc3ccccc3O)O2)cc1]

### Synthesis of methyl (2R,4S,5R,6R)-6-((1R,2R)-1,2-dihydroxy-3-(2-(4-hydroxyphenyl)acetamido)propyl)-4-hydroxy-5-(2-hydroxyacetamido)-2-((4-(prop-2-yn-1-yloxy)benzyl)oxy)tetrahydro-2H-pyran-2-carboxylate (3)

To a solution of methyl (2R,4S,5R,6R)-6-((1R,2R)-3-amino-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylate (0.18 g, 0.37 mmol) and perfluorophenyl 2-(2-hydroxyphenyl)acetate (2, 0.12 g, 0.37 mmol) in N,N-dimethylformamide (5.0 mL), was added DIPEA (0.003 mL, 1.87 mmol) at 0° C. and the reaction mixture was stirred at room temperature for 4 h. The crude residue obtained was purified by flash column chromatography using dichloromethane and methanol as eluent to afford the desired product. Yield: 0.16 g, 69%; LCMS m/z 617.0 [M+H]+.

### Synthesis of (2R,4S,5R,6R)-6-((1R,2R)-1,2-dihydroxy-3-(2-(2-hydroxyphenyl)acetamido)propyl)-4-hydroxy-5-(2-hydroxyacetamido)-2-((4-(prop-2-yn-1-yloxy)benzyl)oxy)tetrahydro-2H-pyran-2-carboxylic acid (Compound 145)

To a stirred solution of methyl (2R,4S,5R,6R)-6-((1R,2R)-1,2-dihydroxy-3-(2-(2-hydroxyphenyl)acetamido)propyl)-4-hydroxy-5-(2-hydroxyacetamido)-2-((4-(prop-2-yn-1-yloxy)benzyl)oxy)tetrahydro-2H-pyran-2-carboxylate (3, 0.15 g, 0.24 mmol) in methanol (5 mL), was added a solution of lithium hydroxide monohydrate (0.006 g, 0.24 mmol) in water (1.0 mL). The reaction mixture was stirred at room temperature for 6 h. The reaction mixture was treated with acidic resin (Dowex 50, H⁺) to reach pH ˜6 and the suspension was filtered. The filtrate was concentrated, and the crude residue obtained was purified by prep HPLC using water and acetonitrile (+0.1% TFA) as eluent to afford the desired product. Yield: 0.035 g, 24%; LCMS m/z 603.35 [M+H]⁺; ¹H NMR (400 MHz, methanol-d₄) δ ¹H NMR (400 MHz, Methanol-d₄) δ 7.91 (d, J=7.6 Hz, 1H), 7.26 (d, J=8.8 Hz, 2H), 7.13-7.05 (m, 2H), 6.95 (d, J=8.8 Hz, 2H), 6.80-6.75 (m, 2H), 4.75-4.71 (m, 3H), 4.42 (d, J=10.8 Hz, 1H), 4.03 (s, 2H), 3.92-3.80 (m, 4H), 3.64 (dd, J=13.6 & 2.8 Hz, 1H), 3.54 (s, 2H), 3.40 (dd, J=8.8 & 2.0 Hz, 1H), 3.31-3.24 (m, 1H), 2.93 (t, J=2.4 Hz, 1H), 2.75 (dd, J=12.4 & 4.0 Hz, 1H), 1.78 (t, J=12.0 Hz, 1H).

### (2R,4S,5R,6R)-6-((1R,2R)-3-(2-(4′-fluoro-[1,1′-biphenyl]-4-yl)acetamido)-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylic acid (Compound 48)

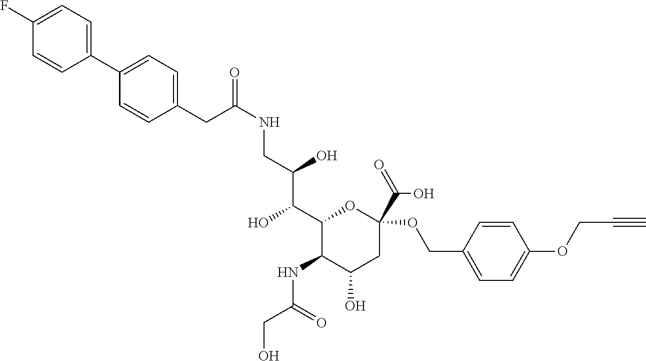 [SMILES: C#CCOc1ccc(CO[C@]2(C(=O)O)C[C@H](O)[C@@H](NC(=O)CO)[C@H]([C@H](O)[C@H](O)CNC(=O)Cc3ccc(-c4ccc(F)cc4)cc3)O2)cc1]

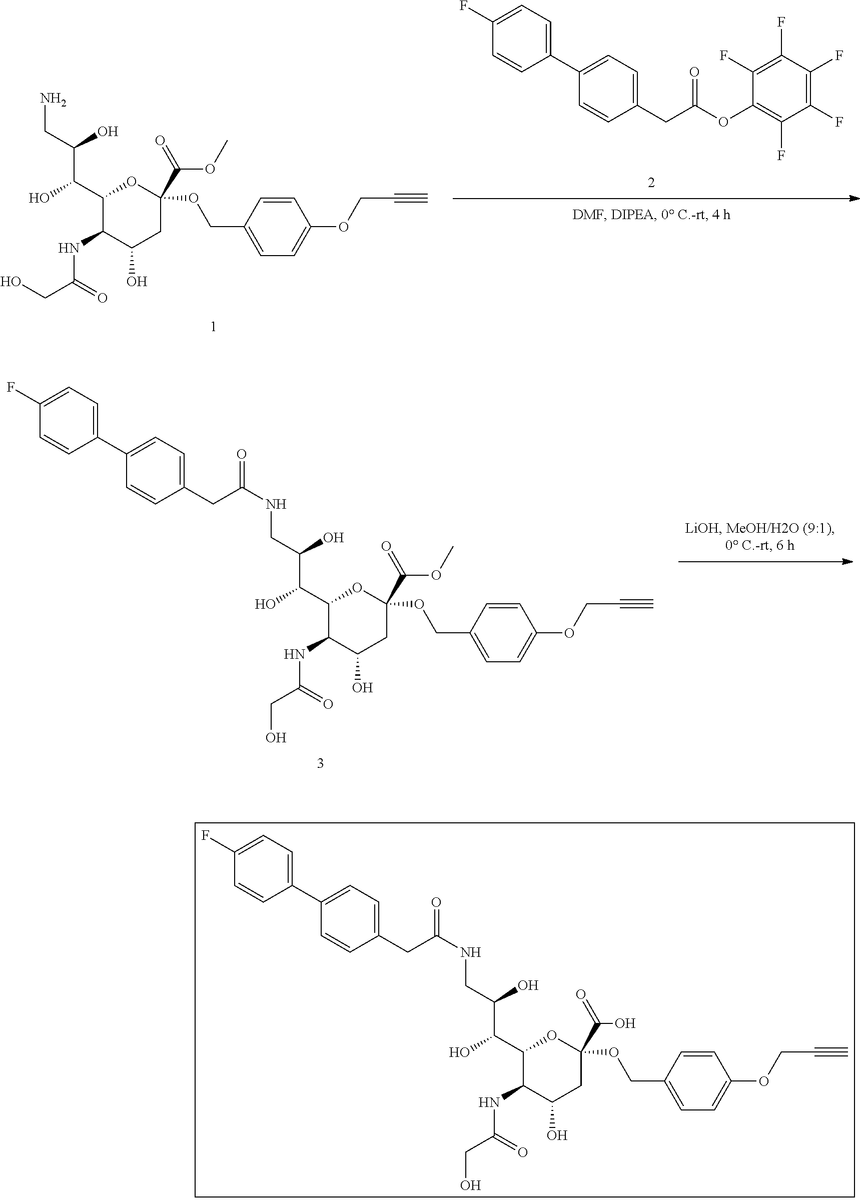 [SMILES: C#CCOc1ccc(CO[C@]2(C(=O)OC)C[C@H](O)[C@@H](NC(=O)CO)[C@H]([C@H](O)[C@H](O)CN)O2)cc1 ; O=C(Cc1ccc(-c2ccc(F)cc2)cc1)Oc1c(F)c(F)c(F)c(F)c1F ; C#CCOc1ccc(CO[C@]2(C(=O)OC)C[C@H](O)[C@@H](NC(=O)CO)[C@H]([C@H](O)[C@H](O)CNC(=O)Cc3ccc(-c4ccc(F)cc4)cc3)O2)cc1 ; C#CCOc1ccc(CO[C@]2(C(=O)O)C[C@H](O)[C@@H](NC(=O)CO)[C@H]([C@H](O)[C@H](O)CNC(=O)Cc3ccc(-c4ccc(F)cc4)cc3)O2)cc1]

### Synthesis of methyl (2R,4S,5R,6R)-6-((1R,2R)-3-(2-(4′-fluoro-[1,1′-biphenyl]-4-yl)acetamido)-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylate (3)

To a stirred solution of methyl (2R,4S,5R,6R)-6-((1R,2R)-3-amino-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylate (0.16 g, 0.33 mmol) and perfluorophenyl 2-(4′-fluoro-[1,1′-biphenyl]-4-yl)acetate (2, 0.105 g, 0.26 mmol) in N,N-dimethylformamide (5.0 mL), was added DIPEA (0.002 mL, 1.03 mmol) at 0° C. and the reaction mixture was stirred at room temperature for 4 h. The crude residue obtained was purified by flash column chromatography using dichloromethane and methanol as eluent to afford the desired product. Yield: 0.11 g, 39%; LCMS m/z 695.25 [M+H]+.

### Synthesis of (2R,4S,5R,6R)-6-((1R,2R)-3-(2-(4′-fluoro-[1,1′-biphenyl]-4-yl)acetamido)-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylic acid (Compound 48)

To a stirred solution of methyl (2R,4S,5R,6R)-6-((1R,2R)-3-(2-(4′-fluoro-[1,1′-biphenyl]-4-yl)acetamido)-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylate (3, 0.11 g, 0.13 mmol) in methanol (5 mL), was added a solution of lithium hydroxide monohydrate (0.012 g, 0.28 mmol) in water (1.0 mL). The reaction mixture was stirred at room temperature for 6 h. After completion, the reaction mixture was treated with acidic resin (Dowex 50, H⁺) to reach pH ˜6 and the suspension was filtered. The filtrate was concentrated. The crude residue obtained was purified by prep HPLC using water and acetonitrile (+0.1% TFA) as eluent to afford the desired product. Yield: 0.029 g, 30%; LCMS m/z 681.5 [M+H]⁺; ¹H NMR (400 MHz, methanol-d₄) δ ¹H NMR (400 MHz, Methanol-d₄) δ 7.91 (d, J=7.6 Hz, 1H), 7.59-7.53 (m, 2H), 7.50 (d, J=8.0 Hz, 2H), 7.37 (d, J=8.0 Hz, 2H), 7.24 (d, J=8.8 Hz, 2H), 7.14 (t, J=8.8 Hz, 2H), 6.91 (d, J=8.8 Hz, 2H), 4.72 (d, J=10.8 Hz, 1H), 4.69 (d, J=2.4 Hz, 2H), 4.39 (d, J=10.8 Hz, 1H), 4.03 (s, 2H), 3.96-3.77 (m, 4H), 3.72 (dd, J=13.6 & 2.8 Hz, 1H), 3.54 (s, 2H), 3.40 (dd, J=8.8 & 2.0 Hz, 1H), 3.30-3.23 (m, 1H), 2.92 (t, J=2.4 Hz, 1H), 2.75 (dd, J=12.4 & 4.0 Hz, 1H), 1.78 (t, J=12.0 Hz, 1H).

### (2R,4S,5R,6R)-6-((1R,2R)-3-(2-(3-fluorophenyl)acetamido)-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylic acid (Compound 167)

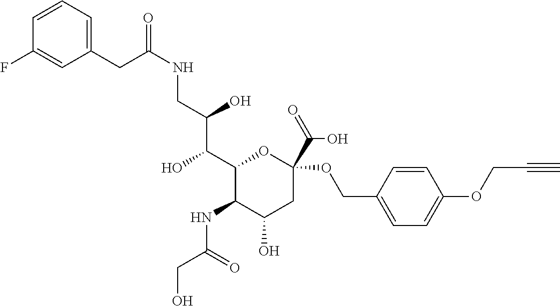 [SMILES: C#CCOc1ccc(CO[C@]2(C(=O)O)C[C@H](O)[C@@H](NC(=O)CO)[C@H]([C@H](O)[C@H](O)CNC(=O)Cc3cccc(F)c3)O2)cc1]

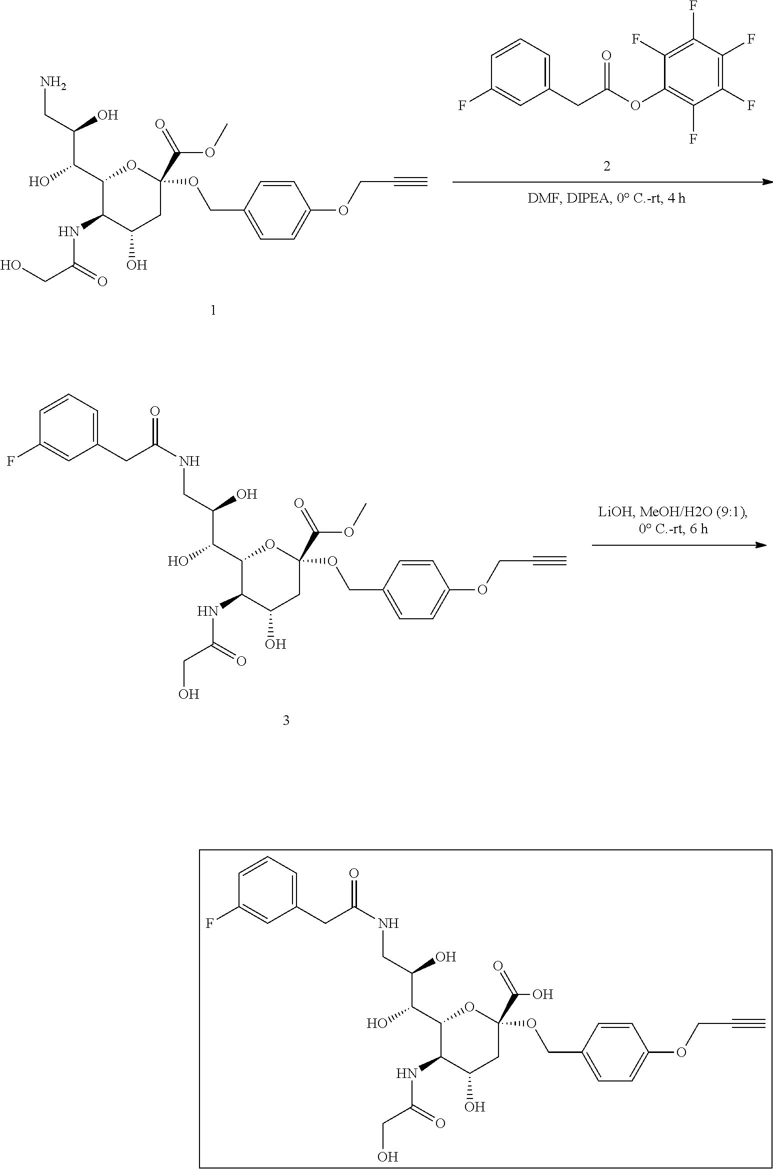 [SMILES: C#CCOc1ccc(CO[C@]2(C(=O)OC)C[C@H](O)[C@@H](NC(=O)CO)[C@H]([C@H](O)[C@H](O)CN)O2)cc1 ; O=C(Cc1cccc(F)c1)Oc1c(F)c(F)c(F)c(F)c1F ; C#CCOc1ccc(CO[C@]2(C(=O)OC)C[C@H](O)[C@@H](NC(=O)CO)[C@H]([C@H](O)[C@H](O)CNC(=O)Cc3cccc(F)c3)O2)cc1 ; C#CCOc1ccc(CO[C@]2(C(=O)O)C[C@H](O)[C@@H](NC(=O)CO)[C@H]([C@H](O)[C@H](O)CNC(=O)Cc3cccc(F)c3)O2)cc1]

### Synthesis of methyl (2R,4S,5R,6R)-6-((1R,2R)-3-(2-(3-fluorophenyl)acetamido)-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylate (3)

To a solution of methyl (2R,4S,5R,6R)-6-((1R,2R)-3-amino-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylate (0.15 g, 0.31 mmol) and perfluorophenyl 2-(3-fluorophenyl)acetate (2, 0.079 g, 0.25 mmol) in N,N-dimethylformamide (5.0 mL), was added DIPEA (0.003 mL, 1.55 mmol) at 0° C. and the reaction mixture was stirred at room temperature for 4 h. The crude residue obtained was purified by flash column chromatography using dichloromethane and methanol as eluent to afford the desired product. Yield: 0.105 g, 54%; LCMS m/z 619.0 [M+H]+.

### Synthesis of (2R,4S,5R,6R)-6-((1R,2R)-3-(2-(3-fluorophenyl)acetamido)-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylic acid (Compound 167)

To a stirred solution of methyl (2R,4S,5R,6R)-6-((1R,2R)-3-(2-(3-fluorophenyl)acetamido)-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylate (3, 0.105 g, 0.17 mmol) in methanol (5 mL), was added a solution of lithium hydroxide monohydrate (0.010 g, 0.17 mmol) in water (0.5 mL). The reaction mixture was stirred at room temperature for 6 h. After completion, the reaction mixture was treated with acidic resin (Dowex 50, H⁺) to reach pH ˜6 and the suspension was filtered. The filtrate was concentrated. The crude residue obtained was purified by prep HPLC using water and acetonitrile (+0.1% TFA) as eluent to afford the desired product. Yield: 0.04 g, 38%; LCMS m/z 605.35 [^{M}+H]⁺; ¹H NMR (400 MHz, Methanol-d₄) δ 7.93 (d, J=7.6 Hz, 1H), 7.31-7.25 (m, 3H), 7.11-7.05 (m, 2H), 6.96-6.92 (m, 3H), 4.74 (d, J=10.8 Hz, 1H), 4.72 (d, J=2.4 Hz, 2H), 4.41 (d, J=10.8 Hz, 1H), 4.03 (s, 2H), 3.96-3.77 (m, 4H), 3.68 (dd, J=14.6 & 2.8 Hz, 1H), 3.55 (s, 2H), 3.40 (dd, J=8.8 & 2.4 Hz, 1H), 3.30-3.22 (m, 1H), 2.93 (t, J=2.4 Hz, 1H), 2.76 (dd, J=12.4 & 4.0 Hz, 1H), 1.78 (t, J=12.4 Hz, 1H).

### (2R,4S,5R,6R)-6-((1R,2R)-3-(2-(4-fluorophenyl)acetamido)-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylic acid (Compound 166)

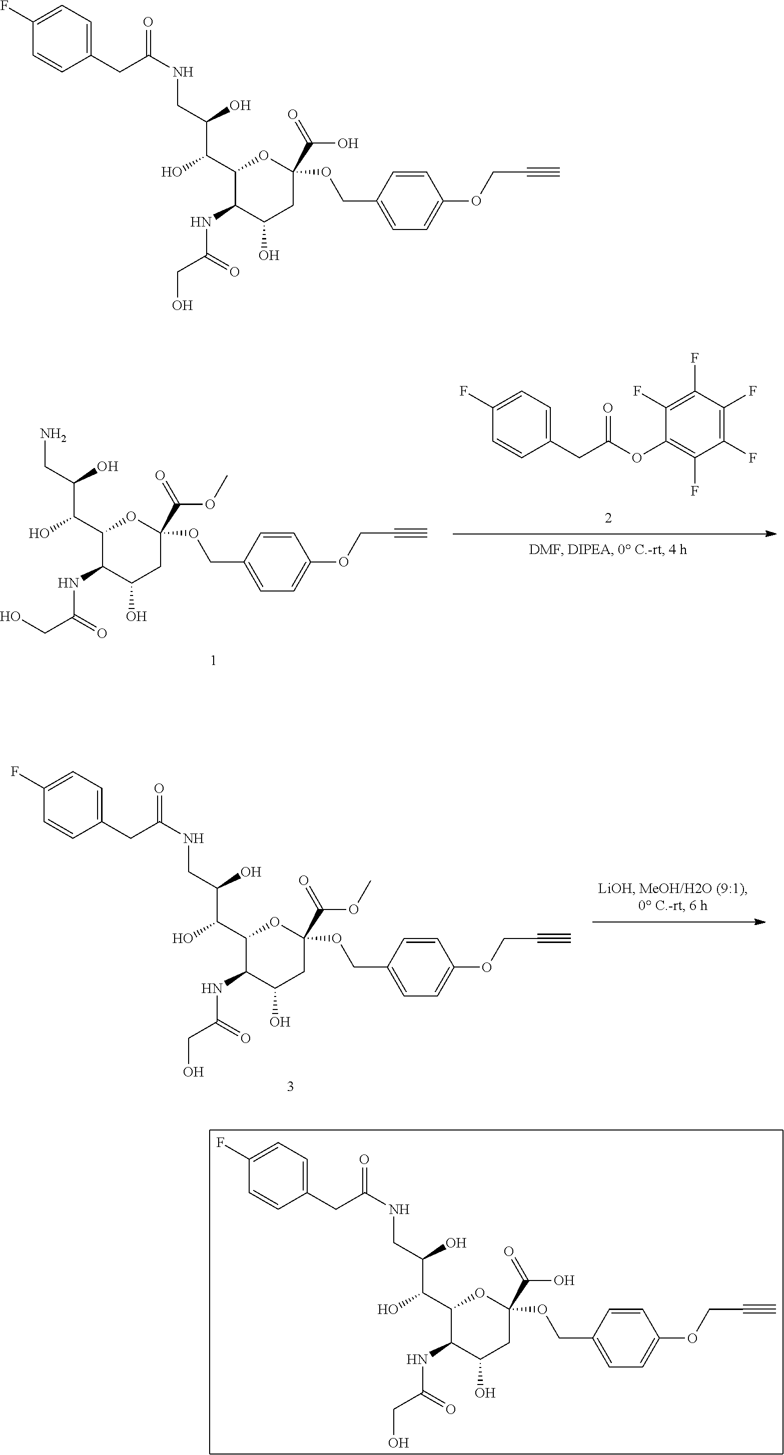 [SMILES: C#CCOc1ccc(CO[C@]2(C(=O)O)C[C@H](O)[C@@H](NC(=O)CO)[C@H]([C@H](O)[C@H](O)CNC(=O)Cc3ccc(F)cc3)O2)cc1 ; C#CCOc1ccc(CO[C@]2(C(=O)OC)C[C@H](O)[C@@H](NC(=O)CO)[C@H]([C@H](O)[C@H](O)CN)O2)cc1 ; O=C(Cc1ccc(F)cc1)Oc1c(F)c(F)c(F)c(F)c1F ; C#CCOc1ccc(CO[C@]2(C(=O)OC)C[C@H](O)[C@@H](NC(=O)CO)[C@H]([C@H](O)[C@H](O)CNC(=O)Cc3ccc(F)cc3)O2)cc1 ; C#CCOc1ccc(CO[C@]2(C(=O)O)C[C@H](O)[C@@H](NC(=O)CO)[C@H]([C@H](O)[C@H](O)CNC(=O)Cc3ccc(F)cc3)O2)cc1]

### Synthesis of methyl (2R,4S,5R,6R)-6-((1R,2R)-3-(2-(4-fluorophenyl)acetamido)-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylate (3)

To a solution of methyl (2R,4S,5R,6R)-6-((1R,2R)-3-amino-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylate (0.15 g, 0.31 mmol) and perfluorophenyl 2-(4-fluorophenyl)acetate (2, 0.099 g, 0.31 mmol) in N,N-dimethylformamide (5.0 mL), was added DIPEA (0.27 mL, 1.55 mmol) at 0° C. and the reaction mixture was stirred at room temperature for 4 h. The crude residue obtained was purified by flash column chromatography using dichloromethane and methanol as eluent to afford the desired product. Yield: 0.12 g, 62%; LCMS m/z 619.0 [M+H]+.

### Synthesis of (2R,4S,5R,6R)-6-((1R,2R)-3-(2-(4-fluorophenyl)acetamido)-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylic acid (Compound 166)

To a stirred solution of methyl (2R,4S,5R,6R)-6-((1R,2R)-3-(2-(4-fluorophenyl)acetamido)-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylate (3, 0.12 g, 0.19 mmol) in methanol (5 mL), was added a solution of lithium hydroxide monohydrate (0.007 g, 0.17 mmol) in water (0.5 mL). The reaction mixture was stirred at room temperature for 6 h. After completion, the reaction mixture was treated with acidic resin (Dowex 50, H⁺) to reach pH ˜6 and the suspension was filtered. The filtrate was concentrated. The crude residue obtained was purified by prep HPLC using water and acetonitrile (+0.1% TFA) as eluent to afford the desired product. Yield: 0.045 g, 38%; LCMS m/z 605.35 [M+H]⁺; ¹H NMR (400 MHz, Methanol-d₄) δ 7.93 (d, J=7.6 Hz, 1H), 7.34-7.23 (m, 4H), 7.12-7.03 (m, 2H), 6.95 (d, J=8.8 Hz, 3H), 4.76 (d, J=10.8 Hz, 1H), 4.72 (d, J=2.4 Hz, 2H), 4.42 (d, J=10.8 Hz, 1H), 4.03 (s, 2H), 3.97-3.77 (m, 4H), 3.67 (dd, J=14.6 & 2.8 Hz, 1H), 3.61 (s, 2H), 3.42 (dd, J=8.8 & 2.4 Hz, 1H), 3.30-3.27 (m, 1H), 2.94 (t, J=2.4 Hz, 1H), 2.76 (dd, J=12.4 & 4.0 Hz, 1H), 1.78 (t, J=12.4 Hz, 1H).

### (2R,4S,5R,6R)-6-((1R,2R)-3-(2-(2-fluorophenyl)acetamido)-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylic acid (Compound 165)

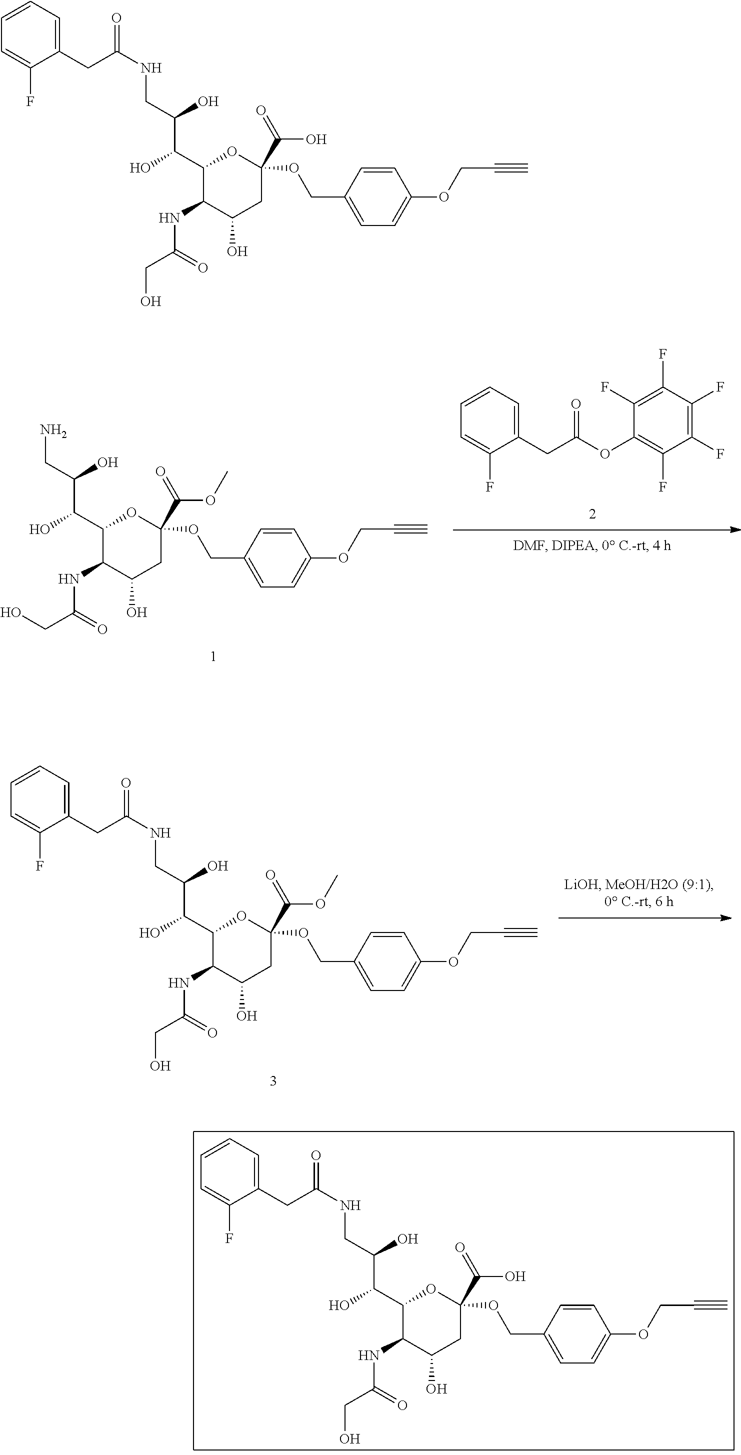 [SMILES: C#CCOc1ccc(CO[C@]2(C(=O)O)C[C@H](O)[C@@H](NC(=O)CO)[C@H]([C@H](O)[C@H](O)CNC(=O)Cc3ccccc3F)O2)cc1 ; C#CCOc1ccc(CO[C@]2(C(=O)OC)C[C@H](O)[C@@H](NC(=O)CO)[C@H]([C@H](O)[C@H](O)CN)O2)cc1 ; O=C(Cc1ccccc1F)Oc1c(F)c(F)c(F)c(F)c1F ; C#CCOc1ccc(CO[C@]2(C(=O)OC)C[C@H](O)[C@@H](NC(=O)CO)[C@H]([C@H](O)[C@H](O)CNC(=O)Cc3ccccc3F)O2)cc1 ; C#CCOc1ccc(CO[C@]2(C(=O)O)C[C@H](O)[C@@H](NC(=O)CO)[C@H]([C@H](O)[C@H](O)CNC(=O)Cc3ccccc3F)O2)cc1]

### Synthesis of methyl (2R,4S,5R,6R)-6-((1R,2R)-3-(2-(2-fluorophenyl)acetamido)-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylate (3)

To a solution of methyl (2R,4S,5R,6R)-6-((1R,2R)-3-amino-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylate (0.12 g, 0.25 mmol) and perfluorophenyl 2-(2-fluorophenyl)acetate (2, 0.079 g, 0.25 mmol) in N,N-dimethylformamide (5.0 mL), was added DIPEA (0.27 mL, 1.55 mmol) at 0° C. and the reaction mixture was stirred at room temperature for 4 h. The crude residue obtained was purified by flash column chromatography using dichloromethane and methanol as eluent to afford the desired product. Yield: 0.09 g, 58%; LCMS m/z 619.2 [M+H]+.

### Synthesis of (2R,4S,5R,6R)-6-((1R,2R)-3-(2-(2-fluorophenyl)acetamido)-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylic acid (Compound 165)

To a stirred solution of methyl (2R,4S,5R,6R)-6-((1R,2R)-3-(2-(2-fluorophenyl)acetamido)-1,2-dihydroxypropyl)-4-hydroxy-5-(2-hydroxyacetamido)-2-((4-(prop-2-yn-1-yloxy)benzyl)oxy)tetrahydro-2H-pyran-2-carboxylate (3, 0.10 g, 0.16 mmol) in methanol (5 mL), was added a solution of lithium hydroxide monohydrate (0.007 g, 0.16 mmol) in water (0.5 mL). The reaction mixture was stirred at room temperature for 6 h. After completion, the reaction mixture was treated with acidic resin (Dowex 50, H⁺) to reach pH ˜6 and the suspension was filtered. The filtrate was concentrated. The crude residue obtained was purified by prep HPLC using water and acetonitrile (+0.1% TFA) as eluent to afford the desired product. Yield: 0.045 g, 42%; LCMS m/z 605.4 [M+H]⁺; ¹H NMR (400 MHz, Methanol-d₄) δ 7.94 (d, J=7.2 Hz, 1H), 7.34-7.23 (m, 4H), 7.12-7.05 (m, 2H), 6.95 (d, J=8.8 Hz, 3H), 4.76 (d, J=10.8 Hz, 1H), 4.72 (d, J=2.4 Hz, 2H), 4.42 (d, J=11.2 Hz, 1H), 4.03 (s, 2H), 3.97-3.77 (m, 4H), 3.68 (dd, J=13.2 & 3.2 Hz, 1H), 3.61 (s, 2H), 3.42 (dd, J=8.8 & 2.4 Hz, 1H), 3.30-3.27 (m, 1H), 2.94 (t, J=2.4 Hz, 1H), 2.76 (dd, J=12.4 & 4.0 Hz, 1H), 1.78 (t, J=12.4 Hz, 1H).

### (2R,4S,5R,6R)-2-((4-(but-3-yn-1-yloxy)benzyl)oxy)-6-((1R,2R)-1,2-dihydroxy-3-(2-(3-morpholinophenyl)acetamido)propyl)-4-hydroxy-5-(2-hydroxyacetamido)tetrahydro-2H-pyran-2-carboxylic acid (Compound 140)

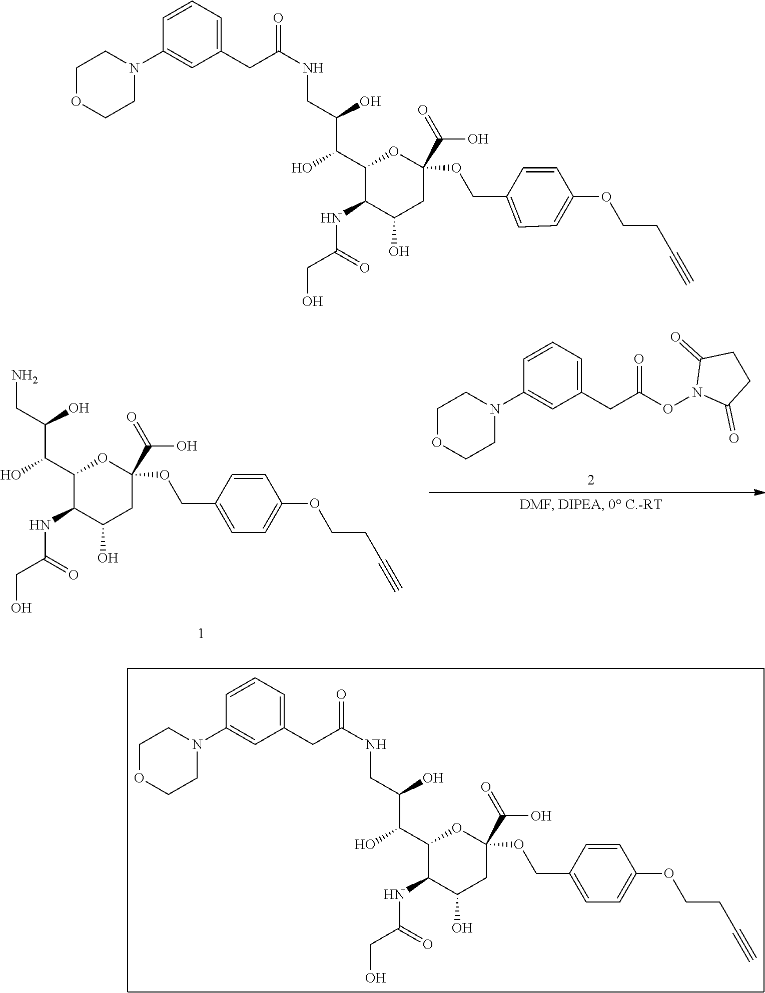 [SMILES: C#CCCOc1ccc(CO[C@]2(C(=O)O)C[C@H](O)[C@@H](NC(=O)CO)[C@H]([C@H](O)[C@H](O)CNC(=O)Cc3cccc(N4CCOCC4)c3)O2)cc1 ; C#CCCOc1ccc(CO[C@]2(C(=O)O)C[C@H](O)[C@@H](NC(=O)CO)[C@H]([C@H](O)[C@H](O)CN)O2)cc1 ; O=C(Cc1cccc(N2CCOCC2)c1)ON1C(=O)CCC1=O ; C#CCCOc1ccc(CO[C@]2(C(=O)O)C[C@H](O)[C@@H](NC(=O)CO)[C@H]([C@H](O)[C@H](O)CNC(=O)Cc3cccc(N4CCOCC4)c3)O2)cc1]

### Synthesis of (2R,4S,5R,6R)-2-((4-(but-3-yn-1-yloxy)benzyl)oxy)-6-((1R,2R)-1,2-dihydroxy-3-(2-(3-morpholinophenyl)acetamido)propyl)-4-hydroxy-5-(2-hydroxyacetamido)tetrahydro-2H-pyran-2-carboxylic acid (Compound 140)

To a stirred solution of (2R,4S,5R,6R)-6-((1R,2R)-3-amino-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (1, 0.150 g, 0.311 mmol) and 2,5-dioxopyrrolidin-1-yl 2-(3-morpholinophenyl)acetate (2, 0.079 g, 0.249 mmol) in N,N-dimethylformamide (4.0 mL), was added DIPEA (0.270 mL, 1.55 mmol) at 0° C. and the reaction mixture was stirred at room temperature for 6 h. After completion, the reaction was concentrated under reduced pressure and the crude residue obtained was purified by prep HPLC using acetonitrile and water (+0.1% TFA) as eluent to afford the desired product. Yield: 0.028 g, 13%; LCMS m/z 686.45 [M+H]+; 1H NMR (400 MHz, methanol-d₄) δ 7.92 (d, J=7.2 Hz, 1H), 7.24-7.21 (m, 3H), 7.02 (bs, 1H), 6.94-6.86 (m, 4H), 4.72 (d, J=10.8 Hz, 1H), 4.42 (d, J=10.8 Hz, 1H), 4.07 (t, J=6.8 Hz, 2H), 4.04 (s, 2H), 4.02-3.76 (m, 8H), 3.65 (dd, J=13.6 Hz & 3.2 Hz, 1H), 3.52 (s, 2H), 3.40 (dd, J=8.8 Hz & 1.2 Hz, 1H), 3.31-3.27 (m, 1H), 3.25-3.15 (m, 4H), 2.76 (dd, J=4.0 Hz, 12.4 Hz, 1H), 2.65-2.61 (m, 2H), 2.32 (t, J=2.4 Hz, 1H), 1.78 (t, J=12.4 Hz, 1H).

### (2R,4S,5R,6R)-6-((1R,2R)-3-(2-(4′-acetamido-[1,1′-biphenyl]-4-yl)acetamido)-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (Compound 50)

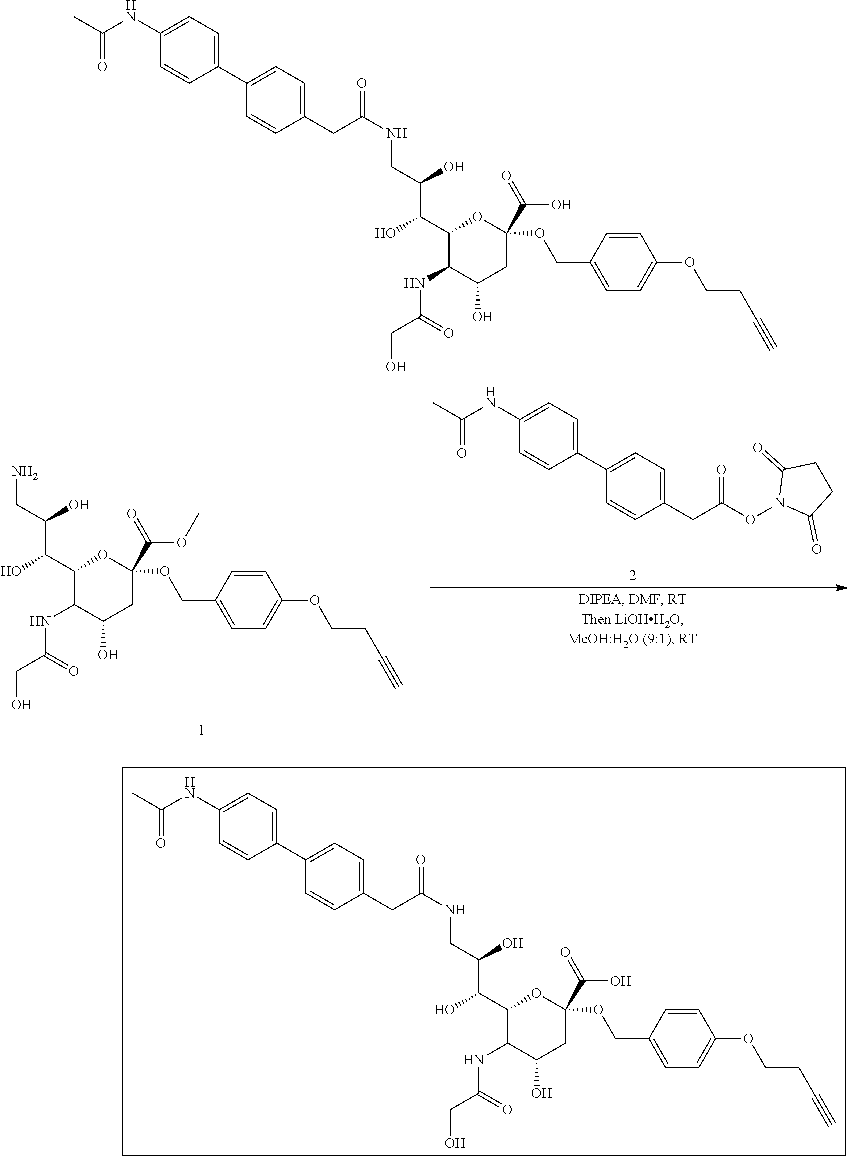

### Synthesis of (2R,4S,5R,6R)-6-((1R,2R)-3-(2-(4′-acetamido-[1,1′-biphenyl]-4-yl)acetamido)-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (Compound 50)

To a stirred solution of methyl (2R,4S,5R,6R)-6-[(1R,2R)-3-amino-1,2-dihydroxypropyl]-2-{[4-(but-3-yn-1-yloxy)phenyl]methoxy}-4-hydroxy-5-(2-hydroxyacetamido)oxane-2-carboxylate (1, 0.20 g, 0.403 mmol) and 2,5-dioxopyrrolidin-1-yl 2-{4′-acetamido-[1,1′-biphenyl]-4-yl}acetate (2, 0.148 g, 0.403 mmol) in N,N-dimethylformamide (4.0 mL), was added DIPEA (0.35 mL, 2.01 mmol) at 0° C. and the reaction mixture was stirred at room temperature for 2 h. After completion the mixture was concentrated and triturated with diethyl ether. The crude was dissolved in methanol (4.0 mL), and a solution of lithium hydroxide monohydrate (0.050 g, 1.21 mmol) in water (0.5 mL) was added. The reaction mixture was stirred at room temperature for 4 h. After completion, the reaction mixture was treated with acidic resin (Dowex 50H⁺) to reach pH ˜6 and the suspension was filtered. The filtrate was concentrated, and the crude residue obtained was purified by prep HPLC using acetonitrile and water (+0.1% TFA) as eluent to afford the desired product. Yield: 0.018 g, 6%; LCMS m/z 734.6 [M+H]+; 1H NMR (400 MHz, methanol-d₄): δ 8.00 (t, J=8.0 Hz, 1H), 7.92 (d, J=7.2 Hz, 1H), 7.61 (d, J=1.6 Hz, 2H), 7.55-7.49 (m, 4H), 7.36 (d, J=8.0 Hz, 2H), 7.22 (d, J=8.4 Hz, 2H), 6.84 (d, J=8.4 Hz, 2H), 4.73 (d, J=10.8 Hz, 1H), 4.40 (d, J=10.8 Hz, 1H), 4.04-3.94 (m, 5H), 3.93-3.78 (m, 5H), 3.57 (m, 2H), 3.42-3.40 (m, 1H), 3.30-3.23 (m, 1H), 2.77 (dd, J=3.2 Hz, 12.0 Hz, 1H), 2.63-2.59 (m, 2H), 2.31 (t, J=2.8 Hz, 1H), 2.14 (s, 3H), 1.76 (m, 1H).

### (2R,4S,5R,6R)-2-((4-(but-3-yn-1-yloxy)benzyl)oxy)-6-((1R,2R)-3-(2-(3-chloro-4-hydroxyphenyl)acetamido)-1,2-dihydroxypropyl)-4-hydroxy-5-(2-hydroxyacetamido)tetrahydro-2H-pyran-2-carboxylic acid (Compound 136)

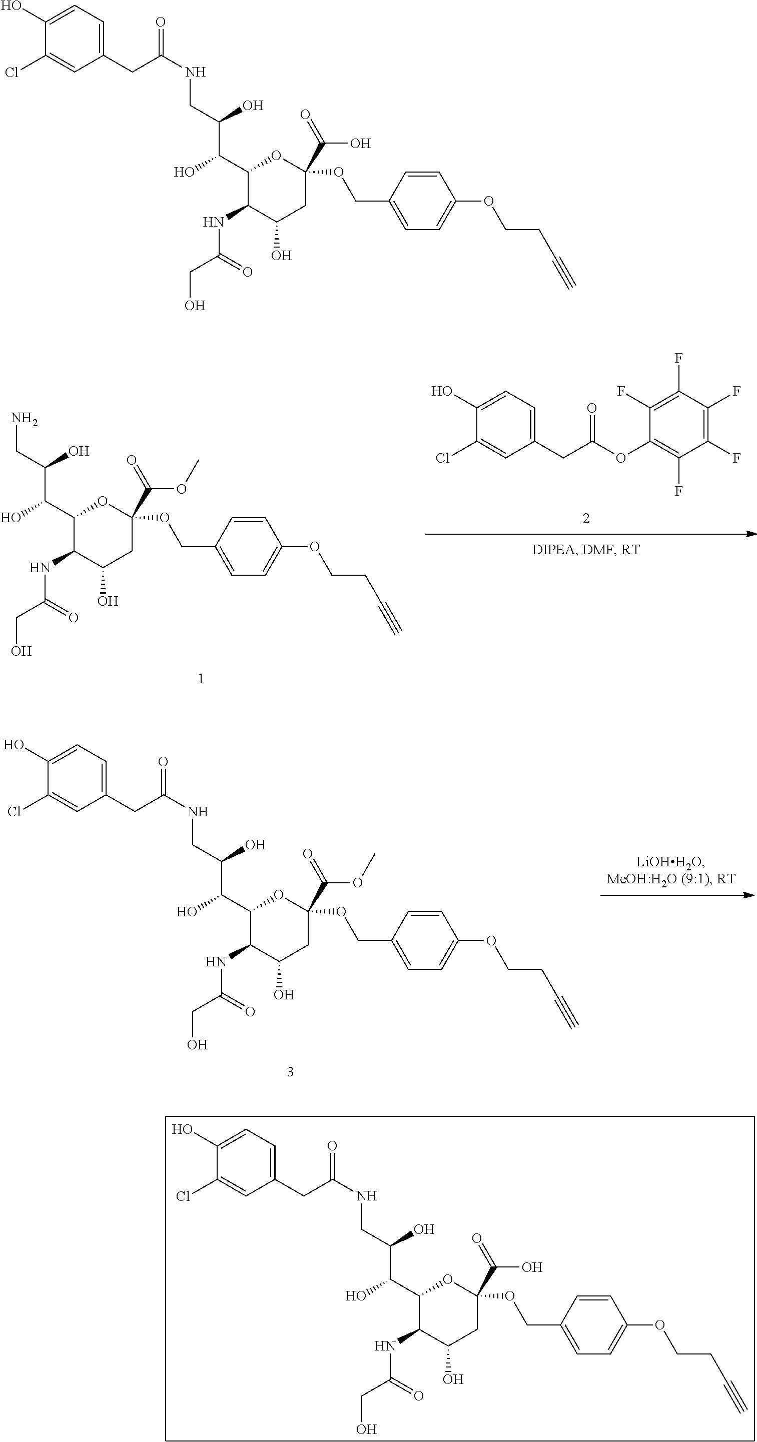 [SMILES: C#CCCOc1ccc(CO[C@]2(C(=O)O)C[C@H](O)[C@@H](NC(=O)CO)[C@H]([C@H](O)[C@H](O)CNC(=O)Cc3ccc(O)c(Cl)c3)O2)cc1 ; C#CCCOc1ccc(CO[C@]2(C(=O)OC)C[C@H](O)[C@@H](NC(=O)CO)[C@H]([C@H](O)[C@H](O)CN)O2)cc1 ; O=C(Cc1ccc(O)c(Cl)c1)Oc1c(F)c(F)c(F)c(F)c1F ; C#CCCOc1ccc(CO[C@]2(C(=O)OC)C[C@H](O)[C@@H](NC(=O)CO)[C@H]([C@H](O)[C@H](O)CNC(=O)Cc3ccc(O)c(Cl)c3)O2)cc1 ; C#CCCOc1ccc(CO[C@]2(C(=O)O)C[C@H](O)[C@@H](NC(=O)CO)[C@H]([C@H](O)[C@H](O)CNC(=O)Cc3ccc(O)c(Cl)c3)O2)cc1]

### Synthesis of methyl (2R,4S,5R,6R)-2-((4-(but-3-yn-1-yloxy)benzyl)oxy)-6-((1R,2R)-3-(2-(3-chloro-4-hydroxyphenyl)acetamido)-1,2-dihydroxypropyl)-4-hydroxy-5-(2-hydroxyacetamido)tetrahydro-2H-pyran-2-carboxylate (3)

To a stirred solution of methyl (2R,4S,5R,6R)-6-((1R,2R)-3-amino-1,2-dihydroxypropyl)-2-((4-(but-3-yn-1-yloxy)benzyl)oxy)-4-hydroxy-5-(2-hydroxyacetamido)tetrahydro-2H-pyran-2-carboxylate (1, 0.20 g, 0.403 mmol) and perfluorophenyl 2-(3-chloro-4-hydroxyphenyl)acetate (2, 0.142 g, 0.403 mmol) in N,N-dimethylformamide (5.0 mL), was added DIPEA (0.35 mL, 2.01 mmol) at 0° C. and the reaction mixture was stirred at room temperature for 4 h. The crude residue was purified by flash column chromatography using ethyl acetate and hexane as eluent to afford the desired product. Yield: 0.150 g, 53%; LCMS m/z 664.85 [M+H]+.

### Synthesis of (2R,4S,5R,6R)-2-((4-(but-3-yn-1-yloxy)benzyl)oxy)-6-((1R,2R)-3-(2-(3-chloro-4-hydroxyphenyl)acetamido)-1,2-dihydroxypropyl)-4-hydroxy-5-(2-hydroxyacetamido)tetrahydro-2H-pyran-2-carboxylic acid (Compound 136)

To a stirred solution of methyl (2R,4S,5R,6R)-2-((4-(but-3-yn-1-yloxy)benzyl)oxy)-6-((1R,2R)-3-(2-(3-chloro-4-hydroxyphenyl)acetamido)-1,2-dihydroxypropyl)-4-hydroxy-5-(2-hydroxyacetamido)tetrahydro-2H-pyran-2-carboxylate (3, 0.15 g, 0.226 mmol) in methanol (5 mL), was added a solution of lithium hydroxide monohydrate (0.028 g, 0.677 mmol) in water (1.0 mL). The reaction mixture was stirred at room temperature for 4 h. After completion, the reaction mixture was treated with acidic resin (Dowex 50, H⁺) to reach pH ˜6 and the suspension was filtered. The filtrate was concentrated under reduced pressure. The crude residue obtained was purified by prep HPLC using water and acetonitrile (+0.1% TFA) as eluent to afford the desired product. Yield: 0.058 g, 39%; LCMS m/z 651.35 [M+H]+;¹H NMR (400 MHz, methanol-d₄) δ 7.25-7.23 (m, 3H), 7.04 (dd, J=8.4 Hz & 2 Hz, 1H), 6.90-6.86 (m, 2H), 6.83 (d, J=8.4 Hz, 1H), 4.74 (d, J=10.8 Hz, 1H), 4.42 (d, J=10.8 Hz, 1H), 4.07 (t, J=6.8 Hz, 2H), 3.96 (s, 2H), 3.94-3.77 (m, 4H), 3.67 (dd, J=13.6 Hz & 2.8 Hz, 1H), 3.42-3.39 (m, 3H), 3.33-3.10 (m, 1H), 2.76 (dd, J=2.0 Hz, 12.4 Hz, 1H), 2.65-2.61 (m, 2H), 2.32 (t, J=2.4 Hz, 1H), 1.83-1.75 (m, 1H).

### (2R,4S,5R,6R)-2-((4-(but-3-yn-1-yloxy)benzyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Compound 124)

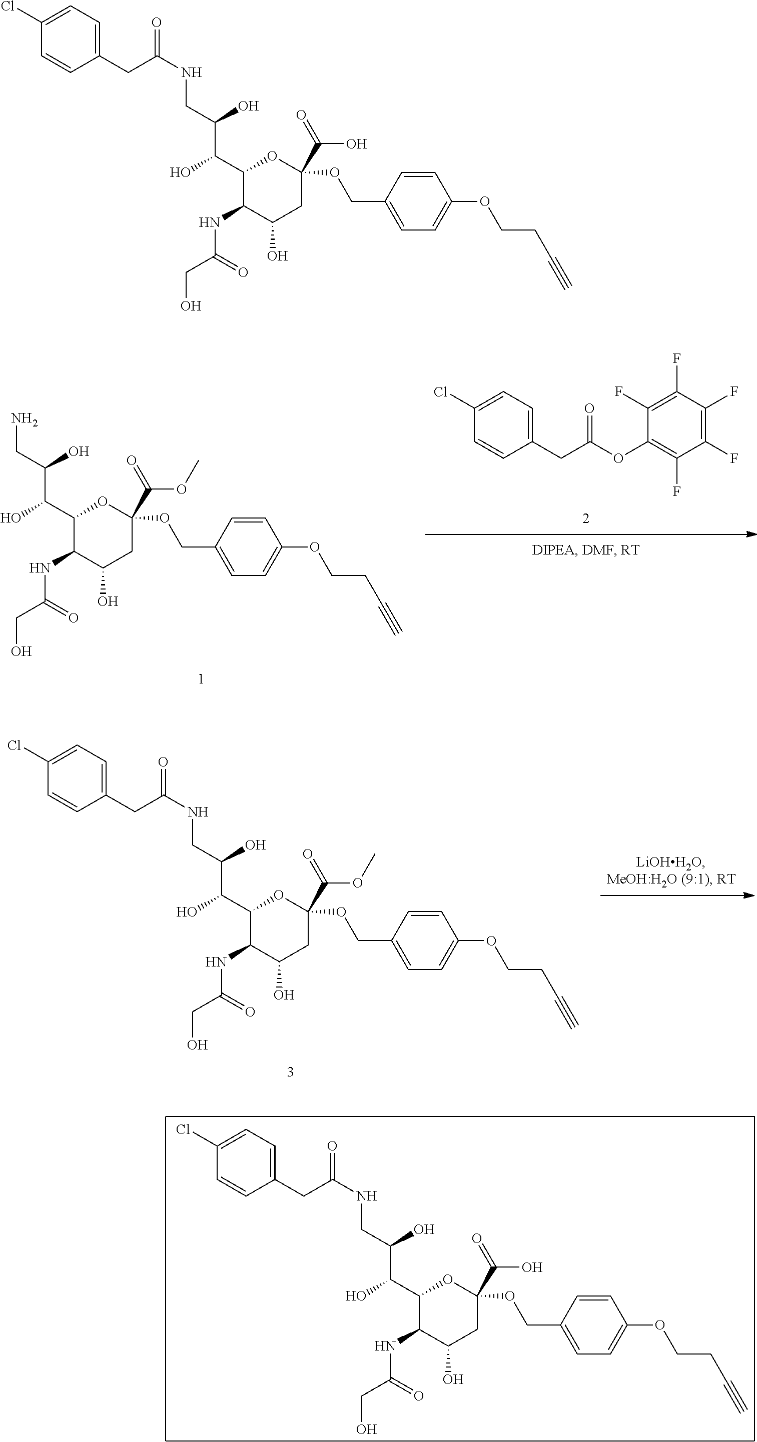 [SMILES: C#CCCOc1ccc(CO[C@]2(C(=O)O)C[C@H](O)[C@@H](NC(=O)CO)[C@H]([C@H](O)[C@H](O)CNC(=O)Cc3ccc(Cl)cc3)O2)cc1 ; C#CCCOc1ccc(CO[C@]2(C(=O)OC)C[C@H](O)[C@@H](NC(=O)CO)[C@H]([C@H](O)[C@H](O)CN)O2)cc1 ; O=C(Cc1ccc(Cl)cc1)Oc1c(F)c(F)c(F)c(F)c1F ; C#CCCOc1ccc(CO[C@]2(C(=O)OC)C[C@H](O)[C@@H](NC(=O)CO)[C@H]([C@H](O)[C@H](O)CNC(=O)Cc3ccc(Cl)cc3)O2)cc1 ; C#CCCOc1ccc(CO[C@]2(C(=O)O)C[C@H](O)[C@@H](NC(=O)CO)[C@H]([C@H](O)[C@H](O)CNC(=O)Cc3ccc(Cl)cc3)O2)cc1]

### Synthesis of methyl (2R,4S,5R,6R)-2-((4-(but-3-yn-1-yloxy)benzyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylate (3)

To a stirred solution of methyl (2R,4S,5R,6R)-6-((1R,2R)-3-amino-1,2-dihydroxypropyl)-2-((4-(but-3-yn-1-yloxy)benzyl)oxy)-4-hydroxy-5-(2-hydroxyacetamido)tetrahydro-2H-pyran-2-carboxylate (0.20 g, 0.402 mmol) and perfluorophenyl 2-(4-chlorophenyl)acetate (2, 0.136 g, 0.402 mmol) in N,N-dimethylformamide (3.0 mL), was added DIPEA (0.340 mL, 2.02 mmol) at 0° C. and the reaction mixture was stirred at room temperature for 2 h. The mixture was diluted with water and ethyl acetate. The organic layer was separated, dried, filtered and the filtrate concentrated. The crude was purified by column chromatography using ethyl acetate and hexane as eluent to afford the desired product. Yield: 0.210 g, 76%; LCMS (ESI) m/z 646.85 [M−1]⁻.

### Synthesis of (2R,4S,5R,6R)-2-((4-(but-3-yn-1-yloxy)benzyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Compound 124)

To a stirred solution of methyl (2R,4S,5R,6R)-2-((4-(but-3-yn-1-yloxy)benzyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylate (3, 0.210 g, 0.324 mmol) in methanol (5.0 mL), was added a solution of lithium hydroxide monohydrate (0.027 g, 0.647 mmol) in water (0.5 mL). The reaction mixture was stirred at room temperature for 4 h. After completion, the reaction mixture was treated with acidic resin (Dowex 50H⁺) to reach pH ˜6 and the suspension was filtered. The filtrate was concentrated, and the crude residue obtained was purified by prep HPLC using acetonitrile and water (+0.1% TFA) as eluent to afford the desired product. Yield: 0.027 g, 13%; LCMS m/z 635.35 [M+H]+; ¹H NMR (400 MHz, methanol-d₄) 7.27-7.23 (m, 6 h), 6.89 (d, J=8.4 Hz, 2H), 4.74 (d, J=10.8 Hz, 2H), 4.41 (d, J=10.8 Hz, 1H), 4.07 (t, J=6.8 Hz, 2H), 4.02 (s, 2H), 3.95-3.90 (m, 1H), 3.88-3.76 (m, 3H), 3.69-3.65 (m, 1H), 3.52 (s, 2H), 3.41-3.39 (m, 1H), 3.27-3.22 (m, 1H), 2.78-2.73 (dd, J=4.0 Hz, 12.4 Hz, 1H), 2.65-2.61 (m, 2H), 2.32 (t, J=2.8 Hz, 1H), 1.78 (t, J=8.8 Hz, 1H).

### (2R,4S,5R,6R)-2-((4-(but-3-yn-1-yloxy)benzyl)oxy)-6-((1R,2R)-1,2-dihydroxy-3-(2-(2′-hydroxy-[1,1′-biphenyl]-3-yl)acetamido)propyl)-4-hydroxy-5-(2-hydroxyacetamido)tetrahydro-2H-pyran-2-carboxylic acid (Compound 46)

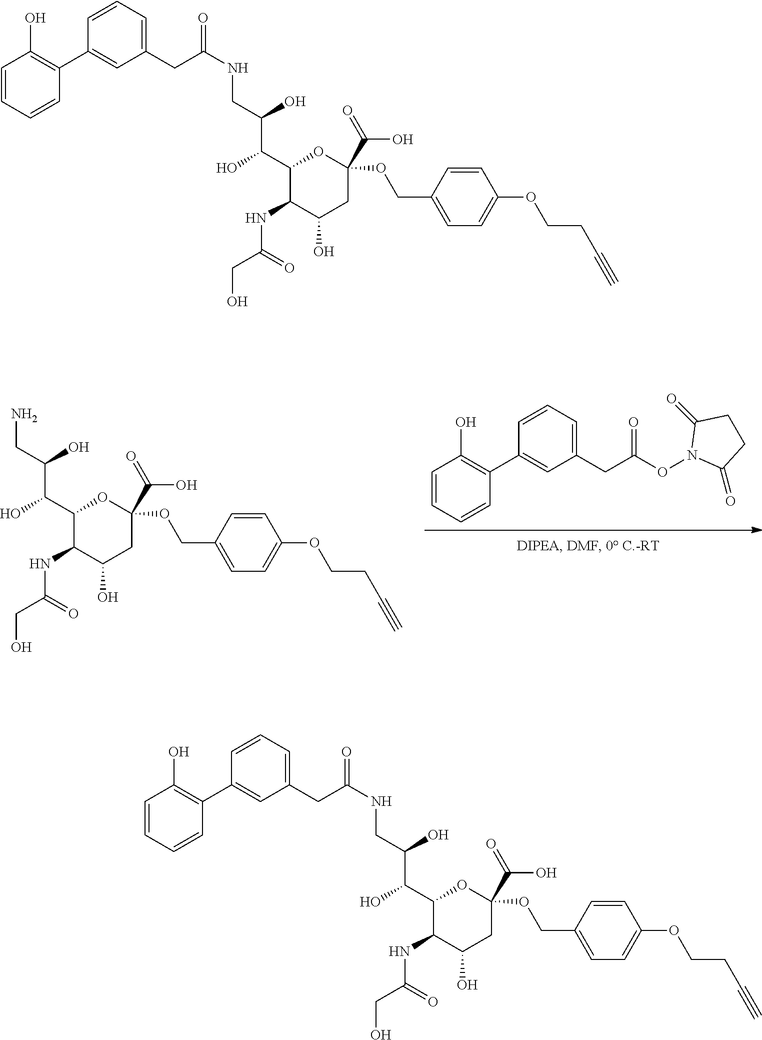 [SMILES: C#CCCOc1ccc(CO[C@]2(C(=O)O)C[C@H](O)[C@@H](NC(=O)CO)[C@H]([C@H](O)[C@H](O)CNC(=O)Cc3cccc(-c4ccccc4O)c3)O2)cc1 ; C#CCCOc1ccc(CO[C@]2(C(=O)O)C[C@H](O)[C@@H](NC(=O)CO)[C@H]([C@H](O)[C@H](O)CN)O2)cc1 ; O=C(Cc1cccc(-c2ccccc2O)c1)ON1C(=O)CCC1=O ; C#CCCOc1ccc(CO[C@]2(C(=O)O)C[C@H](O)[C@@H](NC(=O)CO)[C@H]([C@H](O)[C@H](O)CNC(=O)Cc3cccc(-c4ccccc4O)c3)O2)cc1]

### Synthesis of (2R,4S,5R,6R)-2-((4-(but-3-yn-1-yloxy)benzyl)oxy)-6-((1R,2R)-1,2-dihydroxy-3-(2-(2′-hydroxy-[1,1′-biphenyl]-3-yl)acetamido)propyl)-4-hydroxy-5-(2-hydroxyacetamido)tetrahydro-2H-pyran-2-carboxylic acid (Compound 46)

To a stirred solution of (2R,4S,5R,6R)-6-((1R,2R)-3-amino-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (1, 0.20 g, 0.415 mmol) and 2,5-dioxopyrrolidin-1-yl 2-(2′-hydroxy-[1,1′-biphenyl]-3-yl)acetate (2, 0.108 g, 0.332 mmol) in N,N-dimethylformamide (5.0 mL), was added DIPEA (0.38 mL, 2.07 mmol) at 0° C. and the reaction mixture was stirred at room temperature for 4 h. After completion, the reaction was concentrated. The crude residue obtained was purified by prep HPLC using acetonitrile and water (+0.1% TFA) as eluent to afford the desired product. Yield: 0.080 g, 28%; LCMS m/z 693.4 [M+H]+; 1H NMR (400 MHz, methanol-d₄) 7.86 (d, J=7.6 Hz, 1H), 7.48 (bs, 1H), 7.42-7.40 (m, 1H), 7.31 (t, J=7.6 Hz, 1H), 7.24-7.21 (m, 4H), 7.15-7.10 (m, 1H), 6.89-6.85 (m, 4H), 4.72 (d, J=10.8 Hz, 1H), 4.41 (d, J=10.8 Hz, 1H), 4.05 (t, J=6.8 Hz, 2H), 4.00 (s, 2H), 3.94-3.75 (m, 4H), 3.70-3.65 (m, 1H), 3.59 (s, 2H), 3.40 (dd, J=8.8 Hz & 1.6 Hz, 1H), 3.33-3.31 (m, 1H), 2.75 (dd, J=3.6 Hz, 12.4 Hz, 1H), 2.64-2.60 (m, 2H), 2.31 (t, J=2.8 Hz, 1H), 1.81-1.75 (m, 1H).

### (2R,4S,5R,6R)-6-((1R,2R)-3-(2-(4-acetamidophenyl)acetamido)-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (Compound 168)

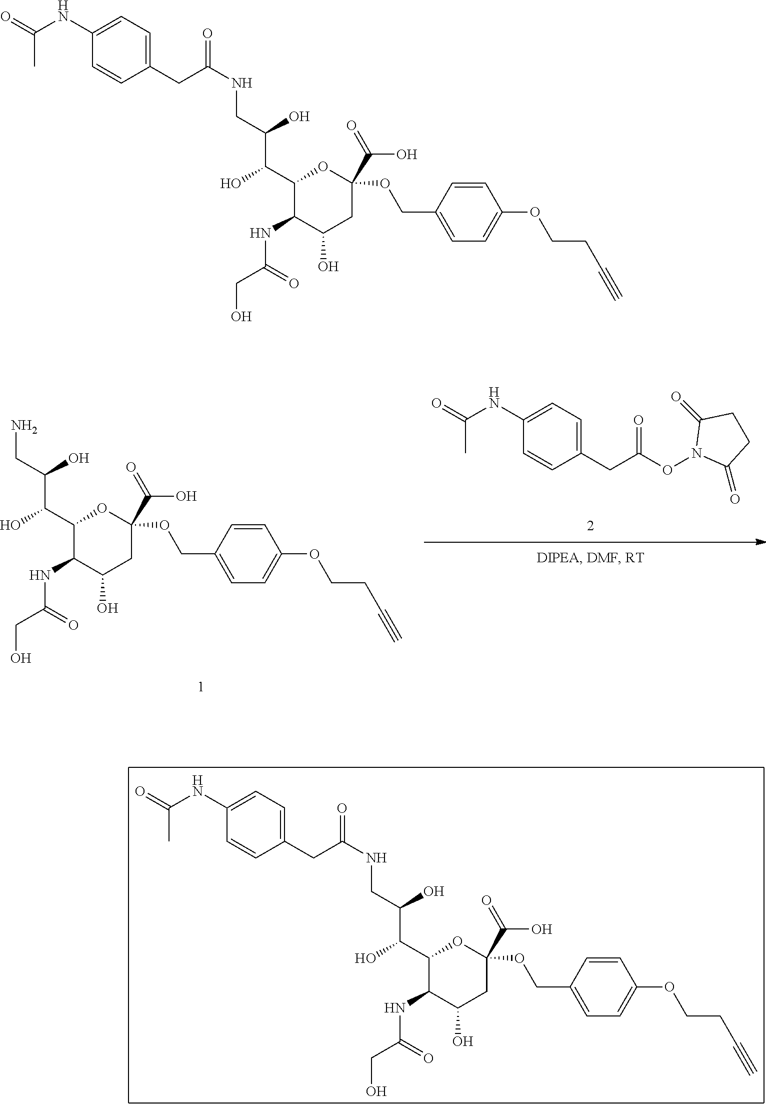 [SMILES: C#CCCOc1ccc(CO[C@]2(C(=O)O)C[C@H](O)[C@@H](NC(=O)CO)[C@H]([C@H](O)[C@H](O)CNC(=O)Cc3ccc(NC(C)=O)cc3)O2)cc1 ; C#CCCOc1ccc(CO[C@]2(C(=O)O)C[C@H](O)[C@@H](NC(=O)CO)[C@H]([C@H](O)[C@H](O)CN)O2)cc1 ; CC(=O)Nc1ccc(CC(=O)ON2C(=O)CCC2=O)cc1 ; C#CCCOc1ccc(CO[C@]2(C(=O)O)C[C@H](O)[C@@H](NC(=O)CO)[C@H]([C@H](O)[C@H](O)CNC(=O)Cc3ccc(NC(C)=O)cc3)O2)cc1]

### Synthesis of (2R,4S,5R,6R)-6-((1R,2R)-3-(2-(4-acetamidophenyl)acetamido)-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (Compound 168)

To a stirred solution of (2R,4S,5R,6R)-6-[(1R,2R)-3-amino-1,2-dihydroxypropyl]-2-{[4-(but-3-yn-1-yloxy)phenyl]methoxy}-4-hydroxy-5-(2-hydroxyacetamido)oxane-2-carboxylic acid (1, 0.20 g, 0.415 mmol) and 2,5-dioxopyrrolidin-1-yl 2-(4-acetamidophenyl)acetate (2, 0.120 g, 0.415 mmol) in N,N-dimethylformamide (4.0 mL), was added DIPEA (0.36 mL, 2.07 mmol) at 0° C. and the reaction mixture was stirred at room temperature for 4 h. After completion, the reaction was concentrated. The crude residue obtained was purified by prep HPLC using acetonitrile and water (+0.1% TFA) as eluent to afford the desired product. Yield: 0.045 g, 17%; LCMS m/z 658.6 [M+H]+; 1H NMR (400 MHz, methanol-d₄) 7.92-7.90 (m, 1H), 7.48 (d, J=8.4 Hz, 2H), 7.25 (d, J=8.8 Hz, 4H), 6.88 (d, J=8.4 Hz, 2H), 4.73 (d, J=10.8 Hz, 1H), 4.42 (d, J=10.8 Hz, 1H), 4.07 (t, J=6.8 Hz, 2H), 4.05 (s, 2H), 3.95-3.75 (m, 4H), 3.66 (dd, J=13.6 Hz & 2.8 Hz, 1H), 3.50 (s, 2H), 3.39 (dd, J=8.8 Hz & 1.6 Hz, 1H), 3.31-3.30 (m, 1H), 2.79 (dd, J=3.2 Hz, 12.4 Hz, 1H), 2.65-2.61 (m, 2H), 2.32 (t, J=2.8 Hz, 1H), 2.09 (s, 3H), 1.74 (t, J=12.4 Hz, 1H).

### (2R,4S,5R,6R)-2-((4-(but-3-yn-1-yloxy)benzyl)oxy)-6-((1R,2R)-3-(2-(3-chlorophenyl)acetamido)-1,2-dihydroxypropyl)-4-hydroxy-5-(2-hydroxyacetamido)tetrahydro-2H-pyran-2-carboxylic acid (Compound 123)

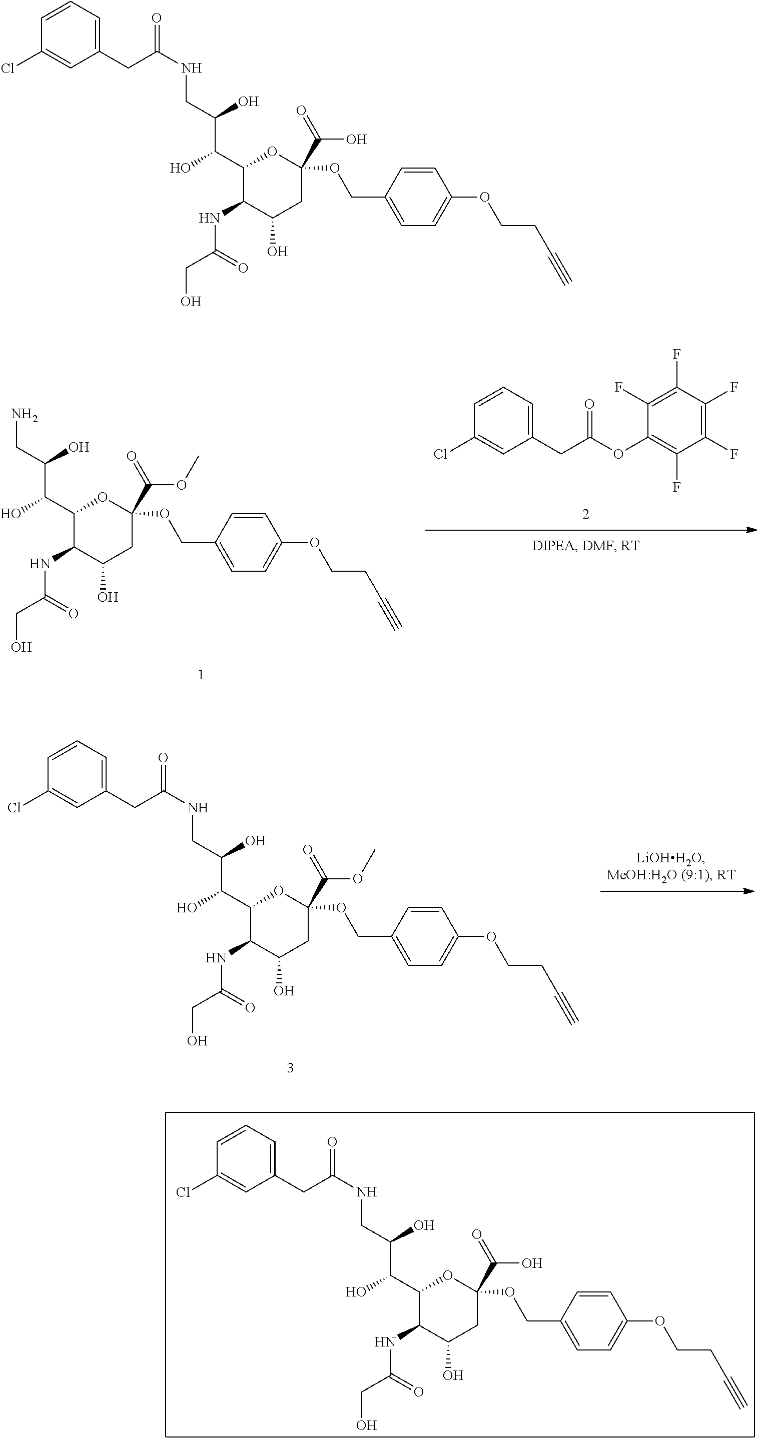 [SMILES: C#CCCOc1ccc(CO[C@]2(C(=O)O)C[C@H](O)[C@@H](NC(=O)CO)[C@H]([C@H](O)[C@H](O)CNC(=O)Cc3cccc(Cl)c3)O2)cc1 ; C#CCCOc1ccc(CO[C@]2(C(=O)OC)C[C@H](O)[C@@H](NC(=O)CO)[C@H]([C@H](O)[C@H](O)CN)O2)cc1 ; O=C(Cc1cccc(Cl)c1)Oc1c(F)c(F)c(F)c(F)c1F ; C#CCCOc1ccc(CO[C@]2(C(=O)OC)C[C@H](O)[C@@H](NC(=O)CO)[C@H]([C@H](O)[C@H](O)CNC(=O)Cc3cccc(Cl)c3)O2)cc1 ; C#CCCOc1ccc(CO[C@]2(C(=O)O)C[C@H](O)[C@@H](NC(=O)CO)[C@H]([C@H](O)[C@H](O)CNC(=O)Cc3cccc(Cl)c3)O2)cc1]

### Synthesis of methyl (2R,4S,5R,6R)-2-((4-(but-3-yn-1-yloxy)benzyl)oxy)-6-((1R,2R)-3-(2-(3-chlorophenyl)acetamido)-1,2-dihydroxypropyl)-4-hydroxy-5-(2-hydroxyacetamido)tetrahydro-2H-pyran-2-carboxylate (3)

To a stirred solution of methyl (2R,4S,5R,6R)-6-((1R,2R)-3-amino-1,2-dihydroxypropyl)-2-((4-(but-3-yn-1-yloxy)benzyl)oxy)-4-hydroxy-5-(2-hydroxyacetamido)tetrahydro-2H-pyran-2-carboxylate (0.30 g, 0.604 mmol) and perfluorophenyl 2-(3-chlorophenyl)acetate (2, 0.203 g, 0.604 mmol) in N,N-dimethylformamide (5.0 mL), was added DIPEA (0.50 mL, 3.02 mmol) at 0° C. and the reaction mixture was stirred at room temperature for 2 h. The mixture was diluted with water and ethyl acetate. The organic layer was separated, dried, filtered and concentrated. The crude was purified by column chromatography using ethyl acetate and hexane as eluent to afford the desired product. Yield: 0.340 g, 86%; LCMS (ESI) m/z 646.75 [M−1]⁻.

### Synthesis of (2R,4S,5R,6R)-2-((4-(but-3-yn-1-yloxy)benzyl)oxy)-6-((1R,2R)-3-(2-(3-chlorophenyl)acetamido)-1,2-dihydroxypropyl)-4-hydroxy-5-(2-hydroxyacetamido)tetrahydro-2H-pyran-2-carboxylic acid (Compound 123)

To a stirred solution of methyl (2R,4S,5R,6R)-2-((4-(but-3-yn-1-yloxy)benzyl)oxy)-6-((1R,2R)-3-(2-(3-chlorophenyl)acetamido)-1,2-dihydroxypropyl)-4-hydroxy-5-(2-hydroxyacetamido)tetrahydro-2H-pyran-2-carboxylate (3, 0.30 g, 0.462 mmol) in methanol (5.0 mL), was added a solution of lithium hydroxide monohydrate (0.038 g, 0.924 mmol) in water (0.5 mL). The reaction mixture was stirred at room temperature for 6 h. After completion, the reaction mixture was treated with acidic resin (Dowex 50H⁺) to reach pH ˜6 and the suspension was filtered. The filtrate was concentrated, and the crude residue obtained was purified by prep HPLC using acetonitrile and water (+0.1% TFA) as eluent to afford the desired product. Yield: 0.114 g, 38%; LCMS m/z 635.35 [M+H]+; ¹H NMR (400 MHz, methanol-d₄) 7.92 (d, J=7.2 Hz, 1H), 7.34 (bs, 1H), 7.27-7.20 (m, 5H), 6.89 (d, J=8.4 Hz, 2H), 4.74 (d, J=10.8 Hz, 2H), 4.42 (d, J=10.8 Hz, 1H), 4.07 (t, J=6.8 Hz, 2H), 4.02 (s, 2H), 3.95-3.90 (m, 1H), 3.88-3.77 (m, 3H), 3.70 (m, 1H), 3.53 (s, 2H), 3.40-3.39 (m, 1H), 3.27-3.22 (m, 1H), 2.78-2.74 (dd, J=3.6 Hz, 12.4 Hz, 1H), 2.65-2.61 (m, 2H), 2.32 (t, J=2.8 Hz, 1H), 1.78 (t, J=12.4 Hz, 1H).

### (2S,4S,5R,6R)-2-((4-(but-3-yn-1-yloxy)benzyl)thio)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Compound 122)

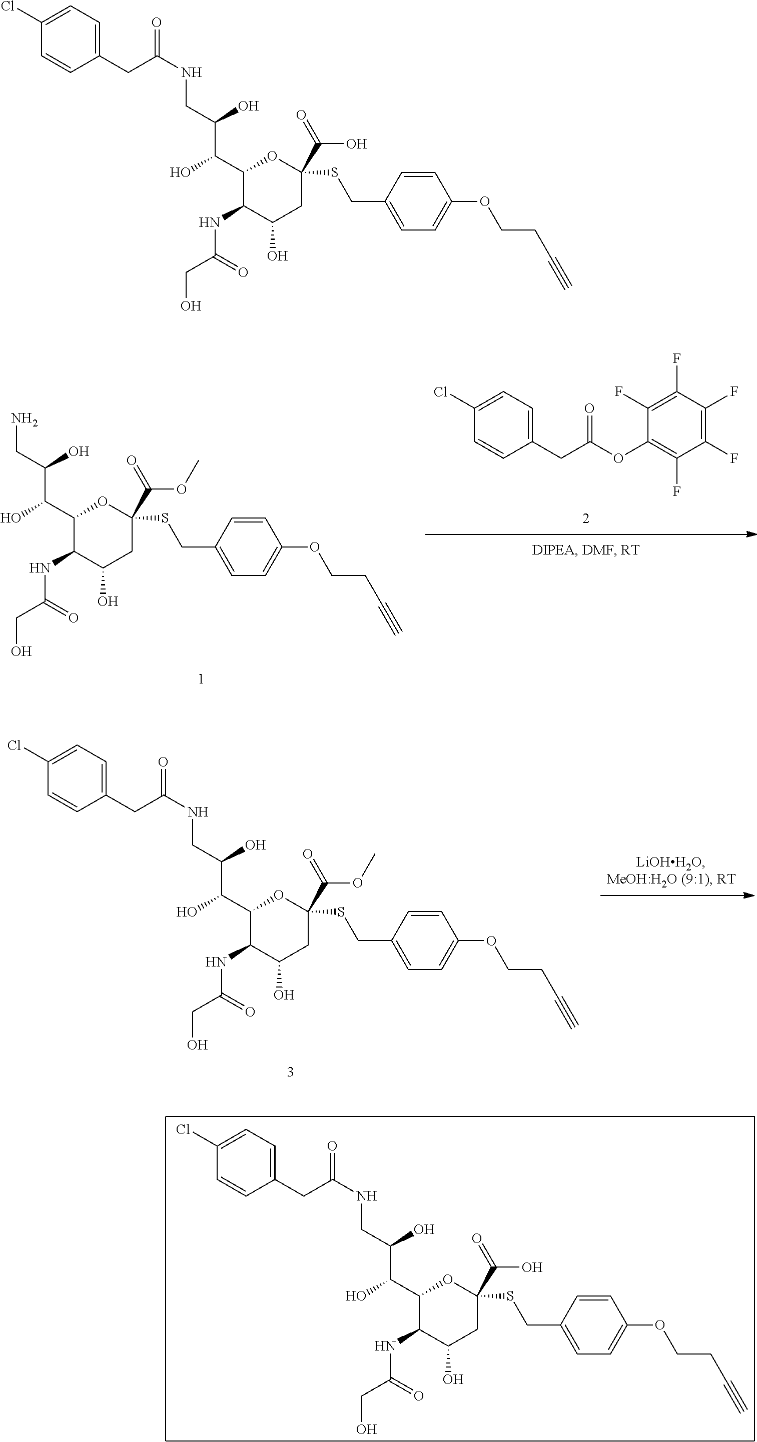 [SMILES: C#CCCOc1ccc(CS[C@]2(C(=O)O)C[C@H](O)[C@@H](NC(=O)CO)[C@H]([C@H](O)[C@H](O)CNC(=O)Cc3ccc(Cl)cc3)O2)cc1 ; C#CCCOc1ccc(CS[C@]2(C(=O)OC)C[C@H](O)[C@@H](NC(=O)CO)[C@H]([C@H](O)[C@H](O)CN)O2)cc1 ; O=C(Cc1ccc(Cl)cc1)Oc1c(F)c(F)c(F)c(F)c1F ; C#CCCOc1ccc(CS[C@]2(C(=O)OC)C[C@H](O)[C@@H](NC(=O)CO)[C@H]([C@H](O)[C@H](O)CNC(=O)Cc3ccc(Cl)cc3)O2)cc1 ; C#CCCOc1ccc(CS[C@]2(C(=O)O)C[C@H](O)[C@@H](NC(=O)CO)[C@H]([C@H](O)[C@H](O)CNC(=O)Cc3ccc(Cl)cc3)O2)cc1]

### Synthesis of methyl (2S,4S,5R,6R)-2-((4-(but-3-yn-1-yloxy)benzyl)thio)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylate (3)

To a stirred solution of methyl (2S,4S,5R,6R)-6-((1R,2R)-3-amino-1,2-dihydroxypropyl)-2-((4-(but-3-yn-1-yloxy)benzyl)thio)-4-hydroxy-5-(2-hydroxyacetamido)tetrahydro-2H-pyran-2-carboxylate (1, 0.20 g, 0.390 mmol) and perfluorophenyl 2-(4-chlorophenyl)acetate (2, 0.131 g, 0.390 mmol) in N,N-dimethylformamide (4.0 mL) was added DIPEA (0.341 mL, 1.95 mmol) at 0° C. and the reaction mixture was stirred at room temperature for 4 h. The crude residue obtained was purified by column chromatography using dichloromethane and methanol as eluent to afford the desired product. Yield: 0.120 g, 46%; LCMS (ESI) m/z 666.6 [M−1]⁻.

### Synthesis of (2S,4S,5R,6R)-2-((4-(but-3-yn-1-yloxy)benzyl)thio)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Compound 122)

To a stirred solution of methyl (2S,4S,5R,6R)-2-((4-(but-3-yn-1-yloxy)benzyl)thio)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylate (3, 0.110 g, 0.165 mmol) in methanol (2.0 mL), was added solution of lithium hydroxide monohydrate (0.012 g, 0.496 mmol) in water (0.2 mL). The reaction mixture was stirred at room temperature for 5 h. After completion, the reaction mixture was treated with acidic (Dowex 50H⁺) to reach pH ˜6 and the suspension was filtered. The filtrate was concentrated, and the crude residue obtained was purified by prep HPLC using water and acetonitrile (+0.1% TFA) as eluent to afford the desired product. Yield: 0.014 g, 13%; LCMS m/z 651.5 [M+H]+; 1H NMR (400 MHz, methanol-d₄): δ 7.26-7.20 (m, 6H), 6.84 (d, J=8.8 Hz, 2H), 4.06 (t, J=6.8 Hz, 2H), 4.02-3.97 (m, 3H), 3.89-3.78 (m, 4H), 3.68-3.62 (m, 2H), 3.51 (bs, 2H), 3.37-3.34 (m, 1H), 3.21-3.15 (m, 1H), 2.83 (dd, J=4.4 Hz, 12.4 Hz, 1H), 2.63 (dt, J=2.4 Hz & 6.8 Hz, 2H), 2.31 (t, J=2.8 Hz, 1H), 1.72 (t, J=11.6 Hz, 1H).

### (2R,4S,5R,6R)-5-amino-2-((4-(but-3-yn-1-yloxy)benzyl)oxy)-6-((1R,2R)-1,2-dihydroxy-3-(2-(4′-hydroxy-[1,1′-biphenyl]-4-yl)acetamido)propyl)-4-hydroxytetrahydro-2H-pyran-2-carboxylic acid (Compound 55)

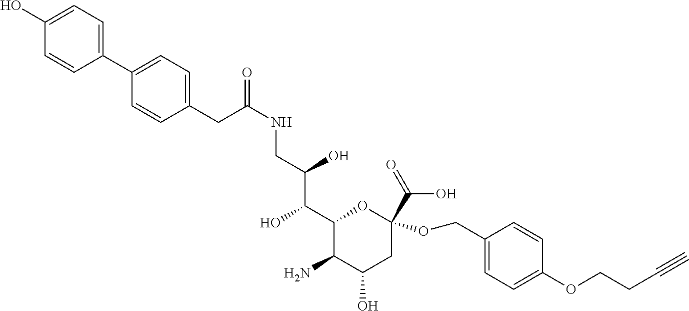 [SMILES: C#CCCOc1ccc(CO[C@]2(C(=O)O)C[C@H](O)[C@@H](N)[C@H]([C@H](O)[C@H](O)CNC(=O)Cc3ccc(-c4ccc(O)cc4)cc3)O2)cc1]

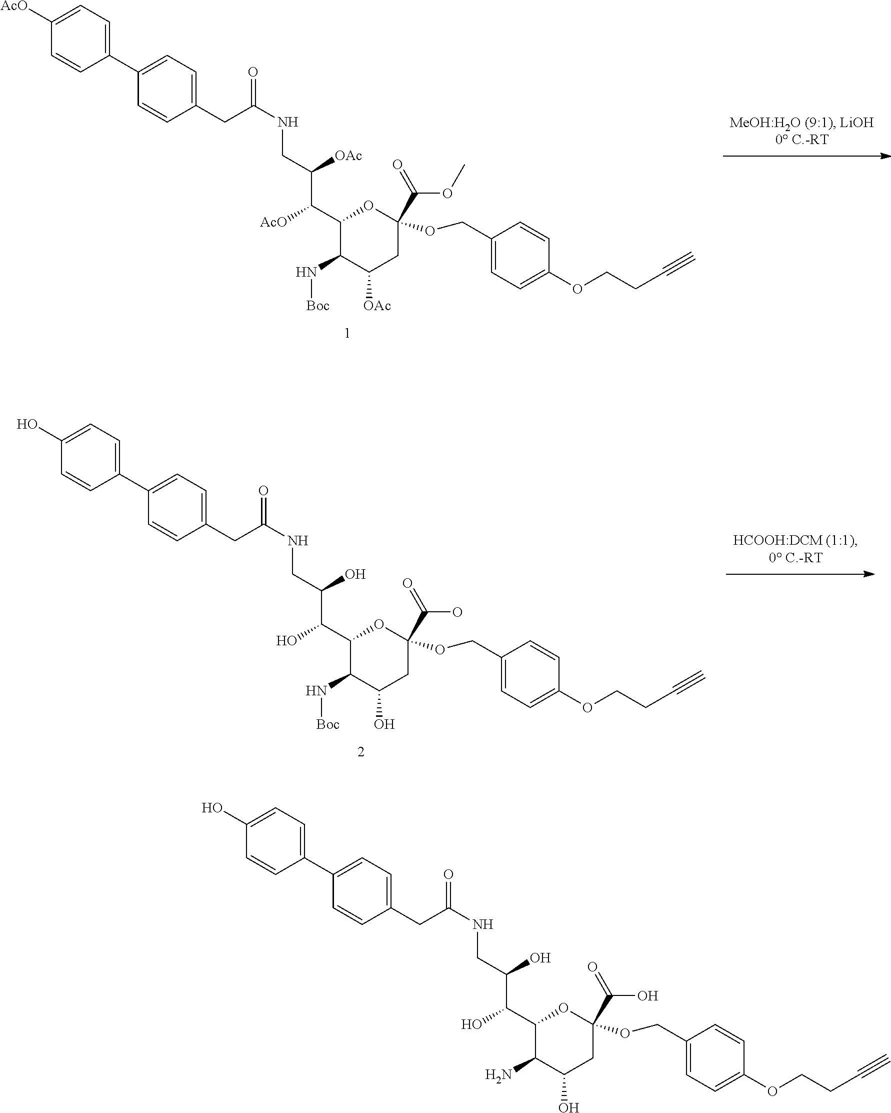 [SMILES: C#CCCOc1ccc(CO[C@]2(C(=O)OC)C[C@H](OC(C)=O)[C@@H](NC(=O)OC(C)(C)C)[C@H]([C@H](OC(C)=O)[C@@H](CNC(=O)Cc3ccc(-c4ccc(OC(C)=O)cc4)cc3)OC(C)=O)O2)cc1 ; C#CCCOc1ccc(CO[C@]2(C(=O)O)C[C@H](O)[C@@H](NC(=O)OC(C)(C)C)[C@H]([C@H](O)[C@H](O)CNC(=O)Cc3ccc(-c4ccc(O)cc4)cc3)O2)cc1 ; C#CCCOc1ccc(CO[C@]2(C(=O)O)C[C@H](O)[C@@H](N)[C@H]([C@H](O)[C@H](O)CNC(=O)Cc3ccc(-c4ccc(O)cc4)cc3)O2)cc1]

### Synthesis of (2R,4S,5R,6R)-2-((4-(but-3-yn-1-yloxy)benzyl)oxy)-5-((tert-butoxycarbonyl)amino)-6-((1R,2R)-1,2-dihydroxy-3-(2-(4′-hydroxy-[1,1′-biphenyl]-4-yl)acetamido)propyl)-4-hydroxytetrahydro-2H-pyran-2-carboxylic acid (2)

To a solution of (1R,2R)-1-((2R,3R,4S,6R)-4-acetoxy-6-((4-(but-3-yn-1-yloxy)benzyl)oxy)-3-((tert-butoxycarbonyl)amino)-6-(methoxycarbonyl)tetrahydro-2H-pyran-2-yl)-3-(2-(4′-acetoxy-[1,1′-biphenyl]-4-yl)acetamido)propane-1,2-diyl diacetate (0.5 g, 0.272 mmol) in methanol (6 mL), was added a solution of lithium hydroxide monohydrate (0.078 g, 1.63 mmol) in water (0.6 mL). The reaction mixture was stirred at room temperature for 6 h. After completion, the reaction mixture was treated with acidic resin (Dowex 50, H⁺) resin to reach pH˜6 and the suspension was filtered. The filtrate was concentrated, and the crude residue obtained was triturated with etherthen dried to afford the desired product as a crude. Yield: 0.380 g, Crude; LCMS m/z 735.10 [M+H]+;

### Synthesis of (2R,4S,5R,6R)-5-amino-2-((4-(but-3-yn-1-yloxy)benzyl)oxy)-6-((1R,2R)-1,2-dihydroxy-3-(2-(4′-hydroxy-[1,1′-biphenyl]-4-yl)acetamido)propyl)-4-hydroxytetrahydro-2H-pyran-2-carboxylic acid (Compound 55)

(2R,4S,5R,6R)-2-((4-(but-3-yn-1-yloxy)benzyl)oxy)-5-((tert-butoxycarbonyl)amino)-6-((1R,2R)-1,2-dihydroxy-3-(2-(4′-hydroxy-[1,1′-biphenyl]-4-yl)acetamido)propyl)-4-hydroxytetrahydro-2H-pyran-2-carboxylic acid (2, 0.15 g, 0.204 mmol) was dissolved in dichloromethane (2 mL) and the resulting solution was cooled to 0° C. in an ice bath. Formic acid (2 mL) was added dropwise, and the mixture was stirred for 10 minutes at this temperature. The ice bath was removed, and the reaction mixture was stirred for 12 h at room temperature. After completion, the reaction mixture was concentrated dried and purified by Prep-HPLC using water and acetonitrile (+0.1% FA) as eluent to afford the desired product. Yield: 0.027 g, 21%; LCMS m/z 635.45 [M+H]+; ¹H NMR (400 MHz, Methanol-d₄) δ 7.44 (d, J=8.0 Hz, 2H), 7.34 (t, J=7.3 Hz, 4H), 7.20 (d, J=8.8 Hz, 2H), 6.81 (d, J=8.4 Hz, 4H), 4.69 (d, J=10.8 Hz, 1H), 4.44 (d, J=10.8 Hz, 1H), 4.03-3.98 (m, 4H), 3.72-3.68 (m, 2H), 3.58 (d, J=4.4 Hz, 2H), 3.50 (dd, J=9.6 & 2.2 Hz, 1H), 3.35-3.30 (m, 1H), 3.11 (t, J=10.2 Hz, 1H), 2.85-2.84 (m, 1H), 2.63-2.59 (m, 2H), 2.31 (t, J=2.6 Hz, 1H), 1.66 (t, J=12.0 Hz, 1H).

### (2R,4S,5R,6R)-6-((1R,2R)-1,2-dihydroxy-3-(2-(4′-hydroxy-[1,1′-biphenyl]-4-yl)acetamido)propyl)-4-hydroxy-5-(2-hydroxyacetamido)-2-(4-(2-hydroxyphenyl)-1H-1,2,3-triazol-1-yl)tetrahydro-2H-pyran-2-carboxylic acid (Compound 54)

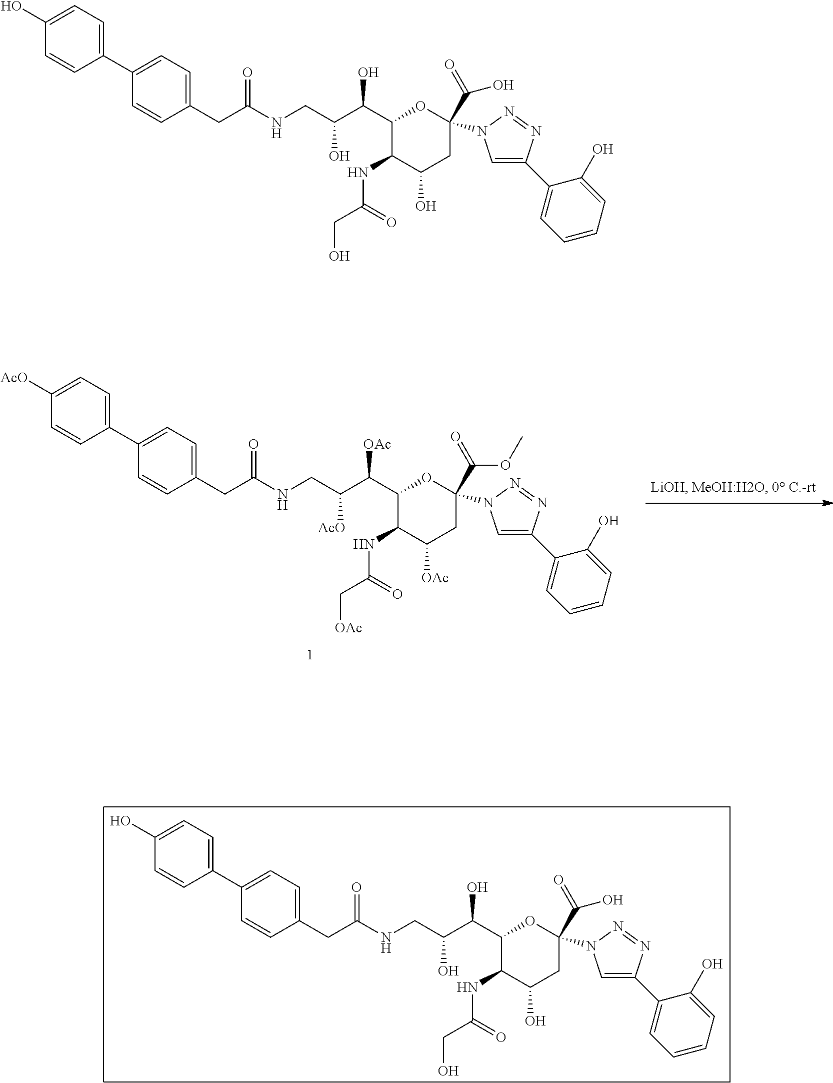 [SMILES: O=C(Cc1ccc(-c2ccc(O)cc2)cc1)NC[C@@H](O)[C@@H](O)[C@@H]1O[C@](C(=O)O)(n2cc(-c3ccccc3O)nn2)C[C@H](O)[C@H]1NC(=O)CO ; COC(=O)[C@@]1(n2cc(-c3ccccc3O)nn2)C[C@H](OC(C)=O)[C@@H](NC(=O)COC(C)=O)[C@H]([C@H](OC(C)=O)[C@@H](CNC(=O)Cc2ccc(-c3ccc(OC(C)=O)cc3)cc2)OC(C)=O)O1 ; O=C(Cc1ccc(-c2ccc(O)cc2)cc1)NC[C@@H](O)[C@@H](O)[C@@H]1O[C@](C(=O)O)(n2cc(-c3ccccc3O)nn2)C[C@H](O)[C@H]1NC(=O)CO]

### Synthesis of (2R,4S,5R,6R)-6-((1R,2R)-1,2-dihydroxy-3-(2-(4′-hydroxy-[1,1′-biphenyl]-4-yl)acetamido)propyl)-4-hydroxy-5-(2-hydroxyacetamido)-2-(4-(2-hydroxyphenyl)-1H-1,2,3-triazol-1-yl)tetrahydro-2H-pyran-2-carboxylic acid (Compound 54)

To a stirred solution of (1R,2R)-1-((2R,3R,4S,6R)-4-acetoxy-3-(2-acetoxyacetamido)-6-(4-(2-hydroxyphenyl)-1H-1,2,3-triazol-1-yl)-6-(methoxycarbonyl)tetrahydro-2H-pyran-2-yl)-3-(2-(4′-acetoxy-[1,1′-biphenyl]-4-yl)acetamido)propane-1,2-diyl diacetate (0.15 g, 0.166 mmol) in methanol (5.0 mL), was added a solution of lithium hydroxide monohydrate (0.023 g, 0.99 mmol) in water (0.5 mL). The reaction mixture was stirred at room temperature for 6 h. After completion, the reaction mixture was treated with acidic resin (Dowex 50H⁺) to reach pH ˜6 and the suspension was filtered. The filtrate was concentrated, and the crude residue obtained was purified by prep HPLC using acetonitrile and water (+0.1% TFA) as eluent to afford the desired product. Yield: 0.042 g, 38%; LCMS m/z 678.40 [M+H]+; ¹H NMR (400 MHz, methanol-d₄)¹H NMR (400 MHz, Methanol-d₄) δ 8.67 (s, 1H), 8.06 (d, J=8.4 Hz, 1H), 7.94 (dd, J=8.0 & 1.6 Hz, 1H), 7.49 (d, J=8.4 Hz, 2H) 7.42 (d, J=14.4 Hz, 2H), 7.33 (d, J=8.0 Hz, 2H), 7.23-7.19 (m, 1H), 6.95-6.91 (m, 2H), 6.86 (d, J=2.8 Hz, 2H), 4.23-4.12 (m, 2H), 4.08-4.01 (m, 3H), 3.98-3.94 (m, 1H), 3.67 (dd, J=16 & 3.2 Hz, 1H), 3.57 (s, 2H), 3.48 (dd, J=7.4 & 4.0 Hz, 1H), 3.38 (dd, J=12.0 & 4.4 Hz, 1H), 3.30-3.28 (m, 1H), 2.36 (t, J=12.0 Hz, 1H).

### (2R,4S,5R,6R)-6-((1R,2R)-3-(2-(4-chlorophenyl)acetamido)-1,2-dihydroxypropyl)-2-((4-ethynylbenzyl)oxy)-4-hydroxy-5-(2-hydroxyacetamido)tetrahydro-2H-pyran-2-carboxylic acid (Compound 114)

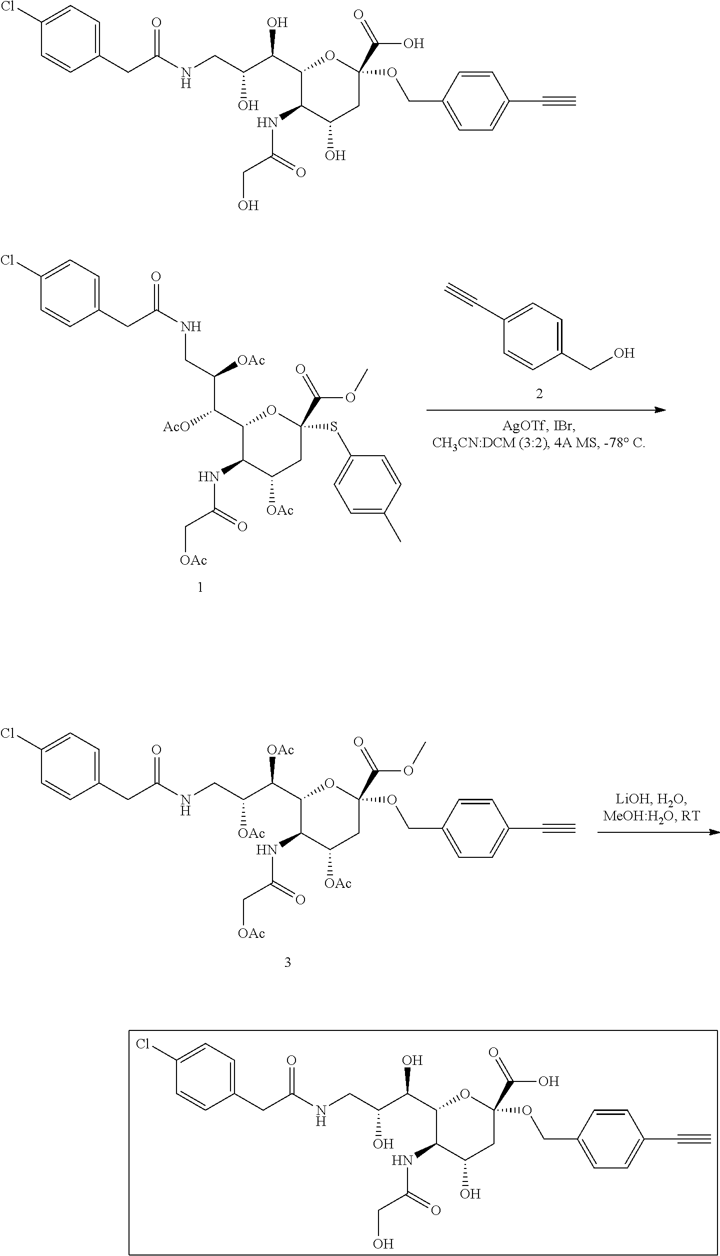

### Synthesis of (1R,2R)-1-((2R,3R,4S,6R)-4-acetoxy-3-(2-acetoxyacetamido)-6-((4-ethynylbenzyl)oxy)-6-(methoxycarbonyl)tetrahydro-2H-pyran-2-yl)-3-(2-(4-chlorophenyl)acetamido)propane-1,2-diyl diacetate (3)

To a solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (0.40 g, 0.523 mmol) and (4-ethynylphenyl)methanol (2, 0.345 g, 2.61 mmol) in anhydrous dichloromethane (12 mL) and anhydrous acetonitrile (16 mL), were added silver(I) trifluoromethanesulfonate (0.269 g, 1.05 mmol) and activated 4 Å powdered molecular sieves (1.00 g). The solution was stirred at room temperature for 1 h under nitrogen atmosphere then cooled to −78° C. Iodine monobromide (0.324 g, 1.57 mmol) in dichloromethane (2.0 mL) was added dropwise to the reaction mixture and was stirred at the same temperature for 2 h. After completion the reaction mixture was quenched with triethyl amine (1.0 mL) and warmed to room temperature. The reaction mixture was filtered, and the filtrate was washed with a saturated solution of sodium bicarbonate and dried over sodium sulfate, filtered and concentrated. The crude product was purified by column chromatography using ethyl acetate and heptane as eluent to afford the desired product. Yield: 0.30 g, (merged peak SM and DP); LCMS m/z 773.10 [M+H]+.

### Synthesis of (2R,4S,5R,6R)-6-((1R,2R)-3-(2-(4-chlorophenyl)acetamido)-1,2-dihydroxypropyl)-2-((4-ethynylbenzyl)oxy)-4-hydroxy-5-(2-hydroxyacetamido)tetrahydro-2H-pyran-2-carboxylic acid (Compound 114)

To a stirred solution of (1R,2R)-1-((2R,3R,4S,6R)-4-acetoxy-3-(2-acetoxyacetamido)-6-((4-ethynylbenzyl)oxy)-6-(methoxycarbonyl)tetrahydro-2H-pyran-2-yl)-3-(2-(4-chlorophenyl)acetamido)propane-1,2-diyl diacetate (3, 0.30 g, 0.388 mmol) in methanol (5.0 mL), was added a solution of lithium hydroxide monohydrate (0.056 g, 2.33 mmol) in water (0.5 mL). The reaction mixture was stirred at room temperature for 6 h. After completion, the reaction mixture was treated with acidic resin (Dowex 50H⁺) to reach pH ˜6 and the suspension was filtered. The filtrate was concentrated, and the crude residue obtained was purified by prep HPLC using acetonitrile and water (+0.1% TFA) as eluent to afford the desired product. Yield: 0.017 g, 7%; LCMS m/z 591.30 [M+H]+; ¹H NMR (400 MHz, methanol-d₄) 7.95 (d, J=7.6 Hz, 1H), 7.45-7.43 (m, 2H), 7.34 (d, J=8.0 Hz, 2H), 7.29 (bs, 4H), 4.84 (d, J=12.0 Hz, 1H), 4.54 (d, J=12.4 Hz, 1H), 4.04 (bs, 2H), 3.94-3.78 (m, 4H), 369-3.65 (m, 1H), 3.54 (s, 2H), 3.48 (bs, 1H), 3.43-3.40 (m, 1H), 3.33-3.30 (m, 1H), 2.83-2.79 (dd, J=4.0 Hz, 12.4 Hz, 1H), 1.81 (t, J=12.4 Hz, 1H).

### (2R,4S,5R,6R)-6-((1R,2R)-1,2-dihydroxy-3-(2-(3-hydroxyphenyl)acetamido)propyl)-2-((4-ethynylbenzyl)oxy)-4-hydroxy-5-(2-hydroxyacetamido)tetrahydro-2H-pyran-2-carboxylic acid (Compound 142)

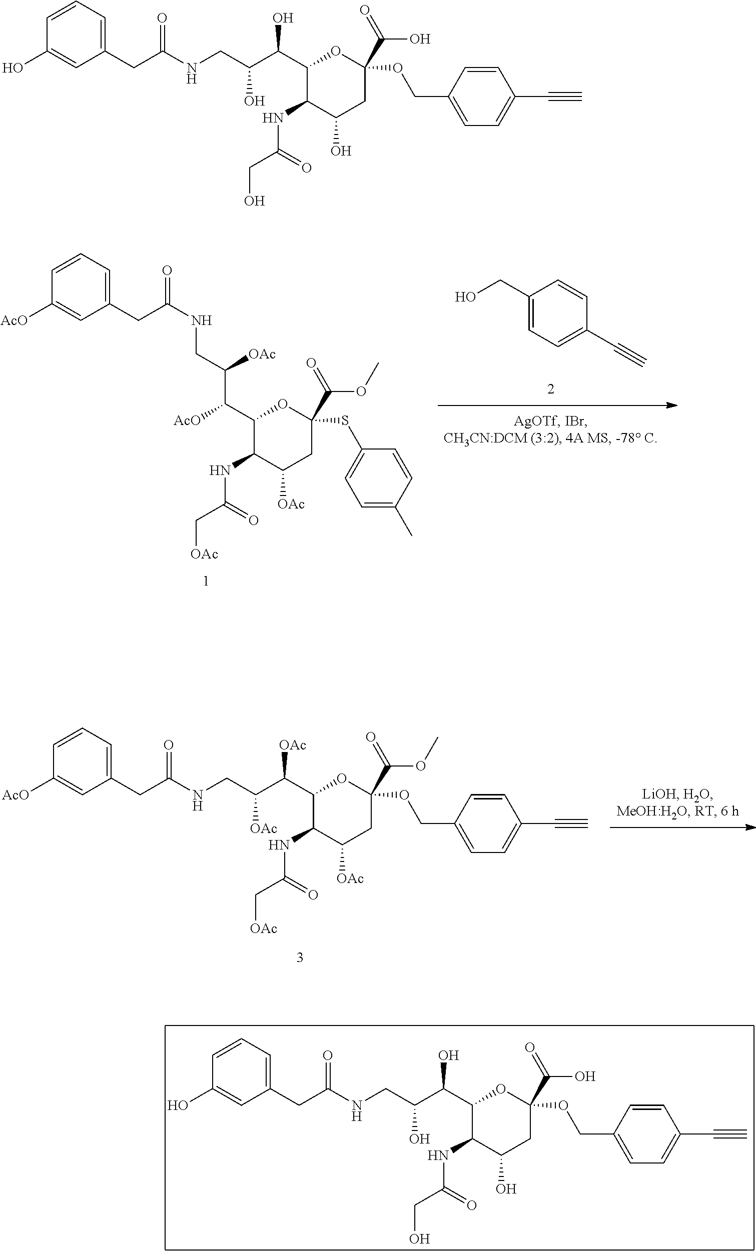

### Synthesis of (1R,2R)-1-((2R,3R,4S,6R)-4-acetoxy-3-(2-acetoxyacetamido)-6-((4-ethynylbenzyl)oxy)-6-(methoxycarbonyl)tetrahydro-2H-pyran-2-yl)-3-(2-(3-acetoxyphenyl)acetamido)propane-1,2-diyl diacetate (3)

To a solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(3-acetoxyphenyl)acetamido)propane-1,2-diyl diacetate (0.50 g, 0.634 mmol) and ((4-ethynylphenyl)methanol (2, 0.419 g, 3.17 mmol) in anhydrous dichloromethane (15 mL) and anhydrous acetonitrile (20 mL), were added silver(I) trifluoromethanesulfonate (0.489 g, 1.90 mmol) and activated 4 Å powdered molecular sieves (1.00 g). The solution was stirred at room temperature for 1 h under nitrogen atmosphere then cooled to −78° C. Iodine monobromide (0.262 g, 1.27 mmol) in dichloromethane (2.0 mL) was added dropwise to the reaction mixture and was stirred at the same temperature for 2 h. After completion the reaction mixture was quenched with triethyl amine (1.0 mL) and warmed to room temperature. The reaction mixture was filtered, and the filtrate was washed with a saturated solution of sodium bicarbonate and dried over sodium sulfate, filtered and concentrated. The crude product was purified by column chromatography using ethyl acetate and heptane as eluent to afford the desired product. Yield: 0.40 g, (merged peak SM and DP); LCMS m/z [M+H]+.

### Synthesis of (2R,4S,5R,6R)-6-((1R,2R)-1,2-dihydroxy-3-(2-(3-hydroxyphenyl)acetamido)propyl)-2-((4-ethynylbenzyl)oxy)-4-hydroxy-5-(2-hydroxyacetamido)tetrahydro-2H-pyran-2-carboxylic acid (Compound 142)

To a stirred solution of (1R,2R)-1-((2R,3R,4S,6R)-4-acetoxy-3-(2-acetoxyacetamido)-6-((4-ethynylbenzyl)oxy)-6-(methoxycarbonyl)tetrahydro-2H-pyran-2-yl)-3-(2-(3-acetoxyphenyl)acetamido)propane-1,2-diyl diacetate (3, 0.40 g, 0.502 mmol) in methanol (5.0 mL), was added a solution of lithium hydroxide monohydrate (0.072 g, 3.01 mmol) in water (1.0 mL). The reaction mixture was stirred at room temperature for 6 h. After completion, the reaction mixture was treated with acidic resin (Dowex 50H⁺) to reach pH ˜6 and the suspension was filtered. The filtrate was concentrated, and the crude residue obtained was purified by prep HPLC using acetonitrile and water (+0.1% FA) as eluent to afford the desired product. Yield: 0.050 g, 17%; LCMS m/z 573.40 [M+H]+; ¹H NMR (400 MHz, methanol-d₄) 7.91 (d, J=7.2 Hz, 1H), 7.43 (d, J=8.4 Hz, 2H), 7.32 (d, J=8.4 Hz, 2H), 7.09 (t, J=8.0 Hz, 1H), 6.76-6.74 (m, 1H), 6.66-6.63 (m, 1H), 4.83 (d, J=12.0 Hz, 1H), 4.53 (d, J=12.4 Hz, 1H), 4.02 (bs, 2H), 3.91-3.77 (m, 4H), 367-3.62 (m, 1H), 3.48-3.46 (m, 3H), 3.41-3.38 (m, 1H), 3.31-3.30 (m, 1H), 2.80-2.76 (dd, J=3.6 Hz, 12.4 Hz, 1H), 1.80 (t, J=12.0 Hz, 1H).

### (2R,4S,5R,6R)-6-((1R,2R)-3-(2-(4-chlorophenyl)acetamido)-1,2-dihydroxypropyl)-4-hydroxy-5-(2-hydroxyacetamido)-2-(4-(oct-7-yn-1-yl)-1H-1,2,3-triazol-1-yl)tetrahydro-2H-pyran-2-carboxylic acid (Compound 119)

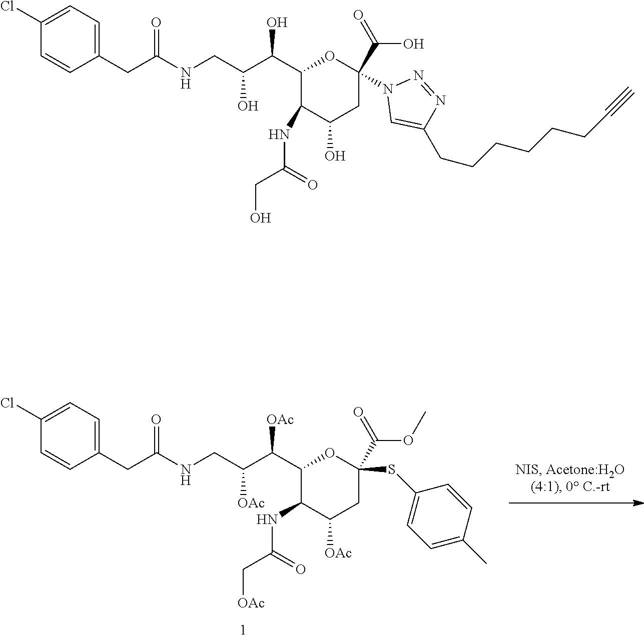 [SMILES: C#CCCCCCCc1cn([C@]2(C(=O)O)C[C@H](O)[C@@H](NC(=O)CO)[C@H]([C@H](O)[C@H](O)CNC(=O)Cc3ccc(Cl)cc3)O2)nn1 ; COC(=O)[C@]1(Sc2ccc(C)cc2)C[C@H](OC(C)=O)[C@@H](NC(=O)COC(C)=O)[C@H]([C@H](OC(C)=O)[C@@H](CNC(=O)Cc2ccc(Cl)cc2)OC(C)=O)O1]

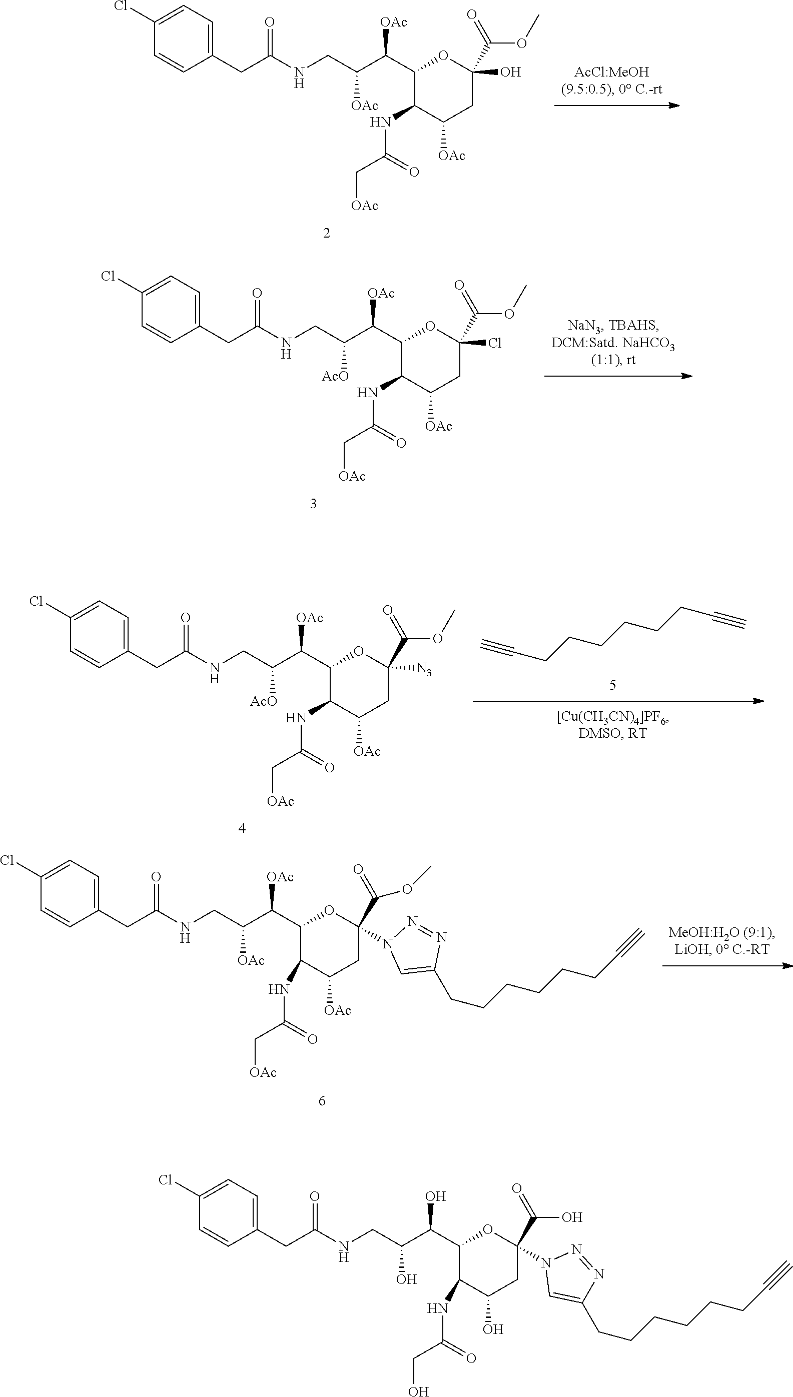

### Synthesis of (1R,2R)-1-((2R,3R, 4S, 6S)-4-acetoxy-3-(2-acetoxyacetamido)-6-hydroxy-6-(methoxycarbonyl)tetrahydro-2H-pyran-2-yl)-3-(2-(4-chlorophenyl)acetamido)propane-1,2-diyl diacetate (2)

A stirred solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (1, 1.0 g, 1.31 mmol) in acetone:water (9:1, 10.0 mL) was cooled to 0° C. To this solution, N-iodosuccinimide (0.58 g, 2.61 mmol) was added and the reaction was maintained at 0° C. for 3 h. After completion, saturated aqueous solution of sodium metabisulfite (10.0 mL) and ethyl acetate (20.0 mL) was added to the reaction mixture and the reaction mixture was stirred for 10 min. The organic layer was separated, and the aqueous phase was washed with ethyl acetate (10 mL). The organic layers were combined and washed sequentially with saturated sodium bicarbonate solution and water. The organic layer was dried and concentrated. The crude residue obtained was purified by column chromatography using ethyl acetate and hexane as eluent to afford the desired product. Yield: 0.60 g, 70%; LCMS (ESI) m/z 660.85 [M+H]+.

### Synthesis of (1R,2R)-1-((2R,3R,4S,6R)-4-acetoxy-3-(2-acetoxyacetamido)-6-chloro-6-(methoxycarbonyl)tetrahydro-2H-pyran-2-yl)-3-(2-(4-chlorophenyl)acetamido)propane-1,2-diyl diacetate (3)

To a stirred solution of (1R,2R)-1-((2R,3R,4S,6S)-4-acetoxy-3-(2-acetoxyacetamido)-6-hydroxy-6-(methoxycarbonyl)tetrahydro-2H-pyran-2-yl)-3-(2-(4-chlorophenyl)acetamido)propane-1,2-diyl diacetate (2, 0.55 g, 0.83 mmol) in acetyl chloride (30.0 mL), was added anhydrous methanol (1.0 mL) dropwise at 0° C. The resulting reaction mixture was stirred at room temperature for 24 h. After completion, the reaction mixture was concentrated to afford the desired product as a crude (crude wt: 0.50 g) which was used without further purification.

### Synthesis of (1R,2R)-1-((2R,3R,4S,6R)-4-acetoxy-3-(2-acetoxyacetamido)-6-azido-6-(methoxycarbonyl)tetrahydro-2H-pyran-2-yl)-3-(2-(4-chlorophenyl)acetamido)propane-1,2-diyl diacetate (4)

To a stirred solution of (1R,2R)-1-((2R,3R,4S,6R)-4-acetoxy-3-(2-acetoxyacetamido)-6-chloro-6-(methoxycarbonyl)tetrahydro-2H-pyran-2-yl)-3-(2-(4-chlorophenyl)acetamido)propane-1,2-diyl diacetate (3, 0.5 g, 0.74 mmol) in dichloromethane (20 ml) was added a solution of tetrabutylammonium hydrogensulfate (0.251 g, 0.74 mmol) and sodium azide (0.24 g, 3.69 mmol) in saturated aqueous solution of sodium bicarbonate (10 mL). The mixture was stirred for 0.5 h. The organic layer was washed successively with 2 M HCl (20 mL), saturated aqueous sodium bicarbonate (20 mL) solution, and brine (30 mL). The organic layer was filtered, and the filtrate was concentrated. The crude residue obtained was purified by silica gel column chromatography using ethyl acetate and hexane as eluent to afford the desired product. 0.3 g; Yield: 0.3 g, 59%; LCMS (ESI) m/z 685.85 [M+H]+.

### Synthesis of (1R,2R)-1-((2R,3R,4S,6R)-4-acetoxy-3-(2-acetoxyacetamido)-6-(methoxycarbonyl)-6-(4-(oct-7-yn-1-yl)-1H-1,2,3-triazol-1-yl)tetrahydro-2H-pyran-2-yl)-3-(2-(4-chlorophenyl)acetamido)propane-1,2-diyl diacetate (6)

To a stirred solution of (1R,2R)-1-((2R,3R,4S,6R)-4-acetoxy-3-(2-acetoxyacetamido)-6-azido-6-(methoxycarbonyl)tetrahydro-2H-pyran-2-yl)-3-(2-(4-chlorophenyl)acetamido)propane-1,2-diyl diacetate (4, 0.23 g, 0.34 mmol) and deca-1,9-diyne (5, 0.14 g, 1.01 mmol) in DMSO (5 mL), were added sequentially 1 M copper(II) pentahydrate sulfate solution (5 ml) and 1 M sodium 5-[(1S)-1,2-dihydroxyethyl]-3-hydroxy-2,4-dioxooxolan-3-ide (5 ml) solution. The reaction mixture was stirred for 12 h at room temperature. The solution was concentrated and the crude was purified by column chromatography using ethyl acetate and hexane as eluent to afford the desired product. Yield: 0.2 g, 72%; LCMS m/z 819.8 [M+H]+

### Synthesis of (2R,4S,5R,6R)-6-((1R,2R)-3-(2-(4-chlorophenyl)acetamido)-1,2-dihydroxypropyl)-4-hydroxy-5-(2-hydroxyacetamido)-2-(4-(oct-7-yn-1-yl)-1H-1,2,3-triazol-1-yl)tetrahydro-2H-pyran-2-carboxylic acid (Compound 119)

To a stirred solution of (1R,2R)-1-((2R,3R,4S,6R)-4-acetoxy-3-(2-acetoxyacetamido)-6-(methoxycarbonyl)-6-(4-(oct-7-yn-1-yl)-1H-1,2,3-triazol-1-yl)tetrahydro-2H-pyran-2-yl)-3-(2-(4-chlorophenyl)acetamido)propane-1,2-diyl diacetate (6, 0.10 g, 0.12 mmol) in methanol (5.0 mL), was added a solution of lithium hydroxide monohydrate (0.017 g, 0.73 mmol) in water (0.5 mL). The reaction mixture was stirred at room temperature for 6 h. After completion, the reaction mixture was treated with acidic resin (Dowex 50H⁺) to reach pH ˜6 and the suspension was filtered. The filtrate was concentrated, and the crude residue obtained was purified by prep HPLC using acetonitrile and water (+0.1% TFA) as eluent to afford the desired product. Yield: 0.013 g, 16%; LCMS m/z 636.50[M+H]+; ¹H NMR (400 MHz, methanol-d₄): δ 8.03 (d, J=8.4 Hz, 1H), 7.98 (s, 1H), 7.32-7.27 (m, 4H), 4.14-4.07 (m, 1H), 4.04-3.86 (m, 5H), 3.64 (dd, J=14.0 & 2.8 Hz, 1H), 3.53 (s, 2H), 3.41 (dd, J=8.8 & 1.2 Hz, 1H), 3.36-3.33 (m, 1H), 3.32-3.22 (m, 1H), 2.70 (t, J=8.0 Hz, 1H), 2.25 (t, J=12.0 Hz, 1H), 2.18-2.14 (m, 3H), 1.71-1.64 (m, 2H), 1.53-1.37 (m, 6H).

### (2R,4S,5R,6R)-6-((1R,2R)-1,2-dihydroxy-3-(2-(3-hydroxyphenyl)acetamido)propyl)-4-hydroxy-5-(2-hydroxyacetamido)-2-(4-(oct-7-yn-1-yl)-1H-1,2,3-triazol-1-yl)tetrahydro-2H-pyran-2-carboxylic acid (Compound 147)

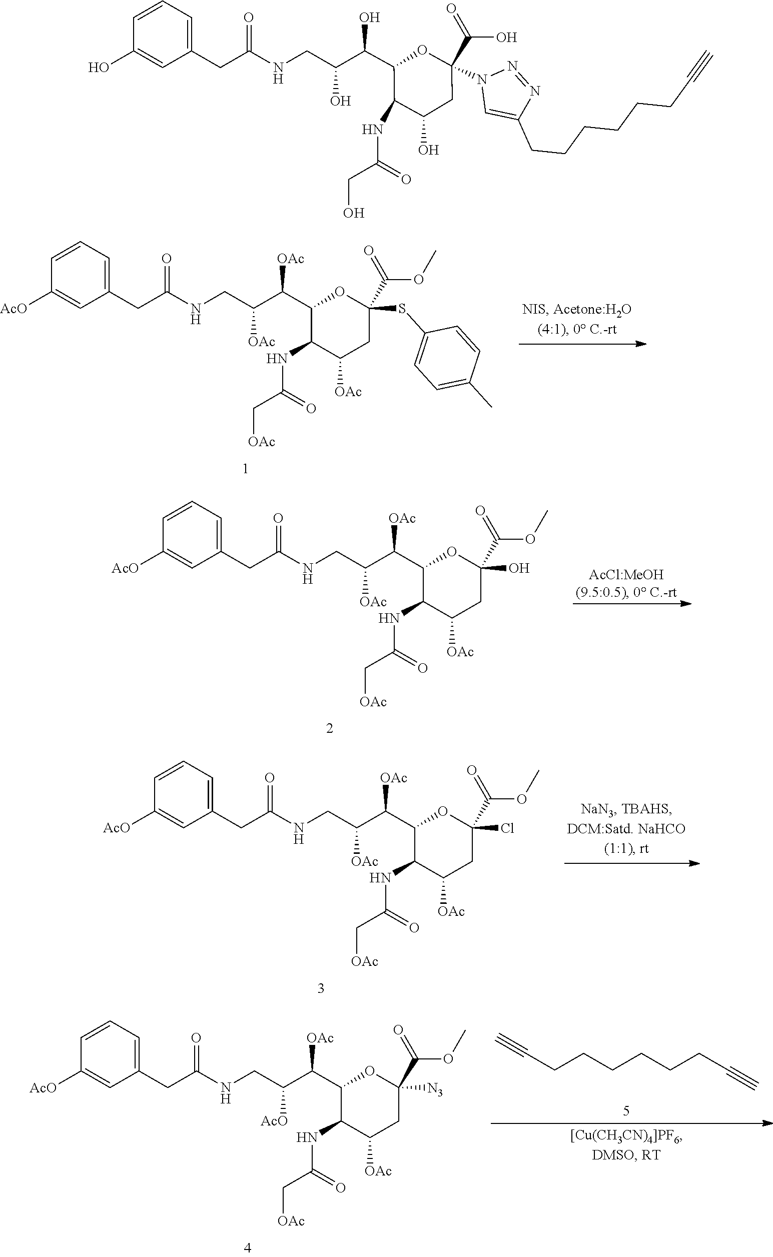

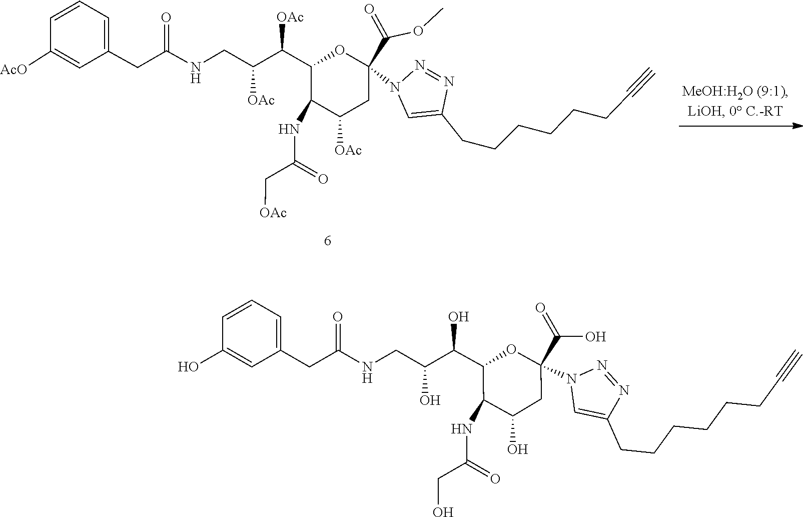 [SMILES: C#CCCCCCCc1cn([C@]2(C(=O)OC)C[C@H](OC(C)=O)[C@@H](NC(=O)COC(C)=O)[C@H]([C@H](OC(C)=O)[C@@H](CNC(=O)Cc3cccc(OC(C)=O)c3)OC(C)=O)O2)nn1 ; C#CCCCCCCc1cn([C@]2(C(=O)O)C[C@H](O)[C@@H](NC(=O)CO)[C@H]([C@H](O)[C@H](O)CNC(=O)Cc3cccc(O)c3)O2)nn1]

### Synthesis of (1R,2R)-1-((2R,3R,4S,6S)-4-acetoxy-3-(2-acetoxyacetamido)-6-hydroxy-6-(methoxycarbonyl)tetrahydro-2H-pyran-2-yl)-3-(2-(3-acetoxyphenyl)acetamido)propane-1,2-diyl diacetate (2)

A stirred solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(3-acetoxyphenyl)acetamido)propane-1,2-diyl diacetate (1, 0.80 g, 1.01 mmol) in acetone:water (9:1, 10.0 mL) was cooled to 0° C. To this solution, N-iodosuccinimide (0.685 g, 3.04 mmol) was added and the reaction was maintained at 0° C. for 3 h. After completion, saturated aqueous solution of sodium metabisulfite (10.0 mL) and ethyl acetate (20.0 mL) was added to the reaction mixture and the reaction mixture was stirred for 10 min. The organic layer was separated, and the aqueous phase was washed with ethyl acetate (10 mL). The organic layers were combined and washed sequentially with saturated sodium bicarbonate solution and water. The organic layer was dried and concentrated. The crude residue obtained was purified by column chromatography using ethyl acetate and hexane as eluent to afford the desired product. Yield: 0.55 g, 79%; LCMS (ESI) m/z 683.0 [M+H]+.

### Synthesis of (1R,2R)-1-((2R,3R,4S,6R)-4-acetoxy-3-(2-acetoxyacetamido)-6-chloro-6-(methoxycarbonyl)tetrahydro-2H-pyran-2-yl)-3-(2-(3-acetoxyphenyl)acetamido)propane-1,2-diyl diacetate (3)

To a stirred solution of (1R,2R)-1-((2R,3R,4S,6S)-4-acetoxy-3-(2-acetoxyacetamido)-6-hydroxy-6-(methoxycarbonyl)tetrahydro-2H-pyran-2-yl)-3-(2-(3-acetoxyphenyl)acetamido)propane-1,2-diyl diacetate (2, 0.40 g, 0.59 mmol) in acetyl chloride (30.0 mL), was added anhydrous methanol (1.0 mL) dropwise at 0° C. The resulting reaction mixture was stirred at room temperature for 24 h. After completion, the reaction mixture was concentrated to afford the desired product as a crude (crude wt: 0.40 g) which was used without further purification.

### Synthesis of (1R,2R)-1-((2R,3R,4S,6R)-4-acetoxy-3-(2-acetoxyacetamido)-6-azido-6-(methoxycarbonyl)tetrahydro-2H-pyran-2-yl)-3-(2-(3-acetoxyphenyl)acetamido)propane-1,2-diyl diacetate (4)

To a stirred solution of (1R,2R)-1-((2R,3R,4S,6R)-4-acetoxy-3-(2-acetoxyacetamido)-6-chloro-6-(methoxycarbonyl)tetrahydro-2H-pyran-2-yl)-3-(2-(3-acetoxyphenyl)acetamido)propane-1,2-diyl diacetate (3, 0.50 g, 0.71 mmol) in dichloromethane (20 ml) was added a solution of tetrabutylammonium hydrogensulfate (0.24 g, 0.71 mmol) and sodium azide (0.23 g, 3.57 mmol) in saturated aqueous solution of sodium bicarbonate (20 mL). The mixture was stirred for 0.5 h. The organic layer was washed successively with 2 M HCl (20 mL), saturated aqueous sodium bicarbonate (20 mL) solution, and brine (30 mL). The organic layer was filtered and the filtrate was concentrated. The crude residue obtained was purified by silica gel column chromatography using ethyl acetate and hexane as eluent to afford the desired product. Yield: 0.25 g, 52%; LCMS (ESI) m/z 707.90 [M+H]+.

### Synthesis of (1R,2R)-1-((2R,3R,4S,6R)-4-acetoxy-3-(2-acetoxyacetamido)-6-(methoxycarbonyl)-6-(4-(oct-7-yn-1-yl)-1H-1,2,3-triazol-1-yl)tetrahydro-2H-pyran-2-yl)-3-(2-(3-acetoxyphenyl)acetamido)propane-1,2-diyl diacetate (6)

To a stirred solution of (1R,2R)-1-((2R,3R,4S,6R)-4-acetoxy-3-(2-acetoxyacetamido)-6-azido-6-(methoxycarbonyl)tetrahydro-2H-pyran-2-yl)-3-(2-(3-acetoxyphenyl)acetamido)propane-1,2-diyl diacetate (4, 0.15 g, 0.21 mmol) and deca-1,9-diyne (5, 0.09 g, 0.63 mmol) in DMSO (5 mL), were added 1 M copper(II) pentahydrate sulfate solution (5 ml) and 1 M sodium 5-[(1S)-1,2-dihydroxyethyl]-3-hydroxy-2,4-dioxooxolan-3-ide (5 ml) solution sequentially. The reaction mixture was stirred for 12 h at room temperature. The solution was concentrated and the crude was purified by column chromatography using ethyl acetate and hexane as eluent to afford the desired product. Yield: 0.1 g, 56%; LCMS m/z 841.9 [M+H]+

### Synthesis of (2R,4S,5R,6R)-6-((1R,2R)-1,2-dihydroxy-3-(2-(3-hydroxyphenyl)acetamido)propyl)-4-hydroxy-5-(2-hydroxyacetamido)-2-(4-(oct-7-yn-1-yl)-1H-1,2,3-triazol-1-yl)tetrahydro-2H-pyran-2-carboxylic acid (Compound 147)

To a stirred solution of (1R,2R)-1-((2R,3R,4S,6R)-4-acetoxy-3-(2-acetoxyacetamido)-6-(methoxycarbonyl)-6-(4-(oct-7-yn-1-yl)-1H-1,2,3-triazol-1-yl)tetrahydro-2H-pyran-2-yl)-3-(2-(3-acetoxyphenyl)acetamido)propane-1,2-diyl diacetate (6, 0.09 g, 0.10 mmol) in methanol (5.0 mL), was added a solution of lithium hydroxide monohydrate (0.026 g, 0.64 mmol) in water (0.5 mL). The reaction mixture was stirred at room temperature for 6 h. After completion, the reaction mixture was treated with acidic resin (Dowex 50H⁺) to reach pH ˜6 and the suspension was filtered. The filtrate was concentrated, and the crude residue obtained was purified by prep HPLC using acetonitrile and water (+0.1% TFA) as eluent to afford the desired product. Yield: 0.007 g, 11%; LCMS m/z 618.50 [M+H]+; ¹H NMR (400 MHz, methanol-d₄): δ 8.02 (d, J=7.6 Hz, 1H), 7.95 (s, 1H), 7.11 (t, J=8.0 Hz, 1H), 6.76-6.74 (m, 2H), 6.66 (dd, J=8.0 & 1.6 Hz, 1H), 4.14-4.04 (m, 3H), 3.97-3.89 (m, 3H), 3.64 (dd, J=14.0 & 2.8 Hz, 1H), 3.47 (s, 2H), 3.42-3.31 (m, 2H), 3.25-3.22 (m, 1H), 2.70 (t, J=8.0 Hz, 2H), 2.25 (t, J=12.0 Hz, 1H), 2.18-2.14 (m, 3H), 1.69-1.65 (m, 2H), 1.51-1.37 (m, 6H).

### (2R,4S,5R,6R)-6-((1R,2R)-3-(2-(1H-indol-5-yl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Compound 6)

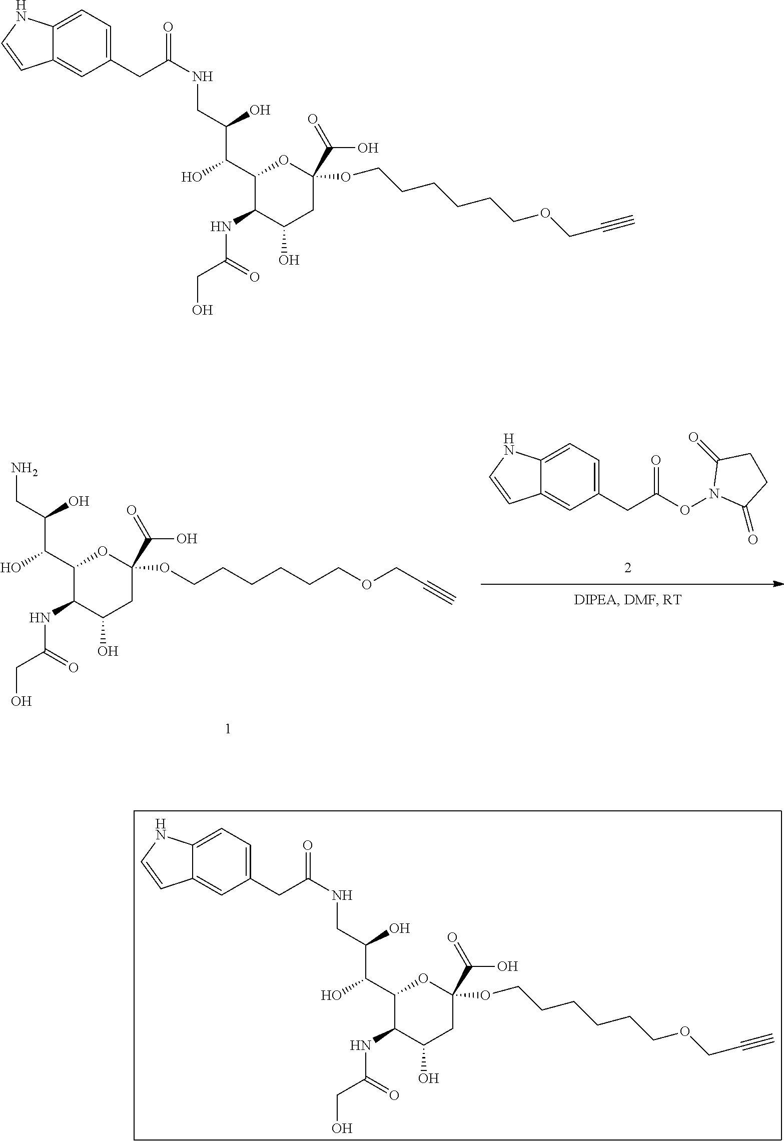 [SMILES: C#CCOCCCCCCO[C@]1(C(=O)O)C[C@H](O)[C@@H](NC(=O)CO)[C@H]([C@H](O)[C@H](O)CNC(=O)Cc2ccc3[nH]ccc3c2)O1 ; C#CCOCCCCCCO[C@]1(C(=O)O)C[C@H](O)[C@@H](NC(=O)CO)[C@H]([C@H](O)[C@H](O)CN)O1 ; O=C(Cc1ccc2[nH]ccc2c1)ON1C(=O)CCC1=O ; C#CCOCCCCCCO[C@]1(C(=O)O)C[C@H](O)[C@@H](NC(=O)CO)[C@H]([C@H](O)[C@H](O)CNC(=O)Cc2ccc3[nH]ccc3c2)O1]

### Synthesis of (2R,4S,5R,6R)-6-((1R,2R)-3-(2-(1H-indol-5-yl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Compound 6)

To a stirred solution of (2R,4S,5R,6R)-6-((1R,2R)-3-amino-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (1, 0.30 g, 0.649 mmol) and 2,5-dioxopyrrolidin-1-yl 2-(1H-indol-5-yl)acetate (2, 0.177 g, 0.649 mmol) in N,N-dimethylformamide (5.0 mL), was added DIPEA (0.565 mL, 3.24 mmol) at 0° C. and reaction mixture was stirred at room temperature for 4 h. After completion the reaction mixture was concentrated, and the crude residue was purified by prep HPLC using acetonitrile and water (+0.1% FA) to afford the desired product. Yield: 0.040 g, 10%; LCMS m/z 620.40 [M+H]+; 1H NMR (400 MHz, methanol-d₄): δ 7.87 (d, J=8.0 Hz, 1H), 7.69-7.66 (m, 1H), 7.48 (d, J=3.2 Hz, 1H), 7.33 (d, J=8.4 Hz, 1H), 7.21-7.20 (m, 1H), 7.04 (dd, J=8.4 Hz & 1.6 Hz, 1H), 6.40-6.39 (m, 1H), 4.11 (d, J=2.4 Hz, 2H), 3.99 (bs, 2H), 3.87-3.62 (m, 6H), 3.60 (bs, 2H), 3.49 (t, J=6.8 Hz, 2H), 3.39-3.33 (m, 2H), 3.26-3.20 (m, 1H), 2.82 (t, J=2.4 Hz, 1H), 2.71 (dd, J=4.0 Hz, 12.4 Hz, 1H), 1.70 (t, J=12.8 Hz, 1H), 1.57-1.48 (m, 4H), 1.35-1.33 (m, 4H).

### (2R,4S,5R,6R)-6-((1R,2R)-3-(2-(1H-indol-6-yl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Compound 5)

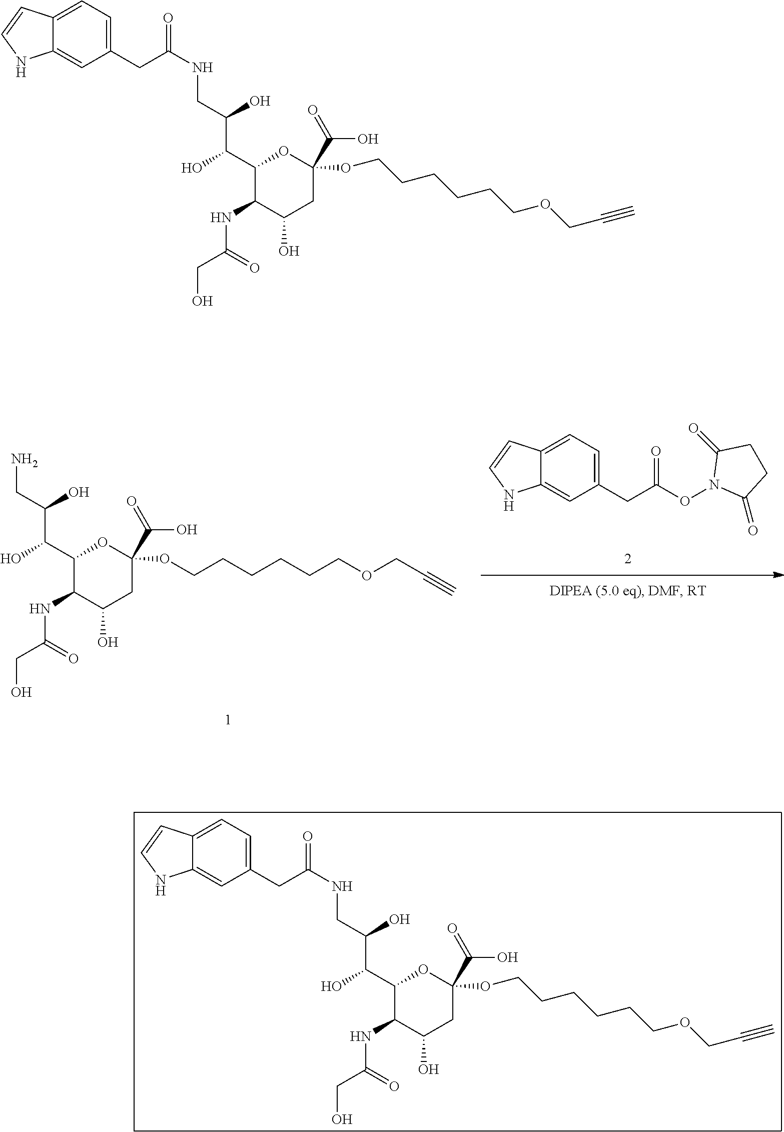 [SMILES: C#CCOCCCCCCO[C@]1(C(=O)O)C[C@H](O)[C@@H](NC(=O)CO)[C@H]([C@H](O)[C@H](O)CNC(=O)Cc2ccc3cc[nH]c3c2)O1 ; C#CCOCCCCCCO[C@]1(C(=O)O)C[C@H](O)[C@@H](NC(=O)CO)[C@H]([C@H](O)[C@H](O)CN)O1 ; O=C(Cc1ccc2cc[nH]c2c1)ON1C(=O)CCC1=O ; C#CCOCCCCCCO[C@]1(C(=O)O)C[C@H](O)[C@@H](NC(=O)CO)[C@H]([C@H](O)[C@H](O)CNC(=O)Cc2ccc3cc[nH]c3c2)O1]

### Synthesis of (2R,4S,5R,6R)-6-((1R,2R)-3-(2-(1H-indol-6-yl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Compound 5)

To a stirred solution of (2R,4S,5R,6R)-6-((1R,2R)-3-amino-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (1, 0.30 g, 0.649 mmol) and 2,5-dioxopyrrolidin-1-yl 2-(1H-indol-6-yl)acetate (2, 0.177 g, 0.649 mmol) in N,N-dimethylformamide (5.0 mL), was added DIPEA (0.565 mL, 3.24 mmol) at 0° C. and reaction mixture was stirred at room temperature for 4 h. Progress of the reaction was monitored by TLC and LCMS and after completion the reaction mixture was concentrated under reduced pressure to get crude which was purified by prep HPLC using 100% ACN in water with 0.1% FA as buffer to afford (2R,4S,5R,6R)-6-((1R,2R)-3-(2-(1H-indol-6-yl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Compound 5) as a white solid. Yield: 0.015 g, 4%; LCMS m/z 620.40 [M+H]+; ¹H NMR (400 MHz, methanol-d₄): δ 7.49 (d, J=8.0 Hz, 1H), 7.32 (s, 1H), 7.19 (d, J=3.2 Hz, 1H), 6.95 (dd, J=8.0 Hz & 1.2 Hz, 1H), 6.40-6.39 (m, 1H), 4.11 (d, J=2.4 Hz, 2H), 3.99 (bs, 2H), 3.87-3.65 (m, 6H), 3.62 (bs, 2H), 3.49 (t, J=6.8 Hz, 2H), 3.41-3.34 (m, 2H), 3.30-3.22 (m, 1H), 2.80 (t, J=2.4 Hz, 1H), 2.72 (dd, J=3.2 Hz & 12.4 Hz, 1H), 1.69 (t, J=12.8 Hz, 1H), 1.57-1.50 (m, 4H), 1.35-1.30 (m, 4H).

### (2R,4S,5R,6R)-6-((1R,2R)-3-(2-(4-cyanophenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Compound 162)

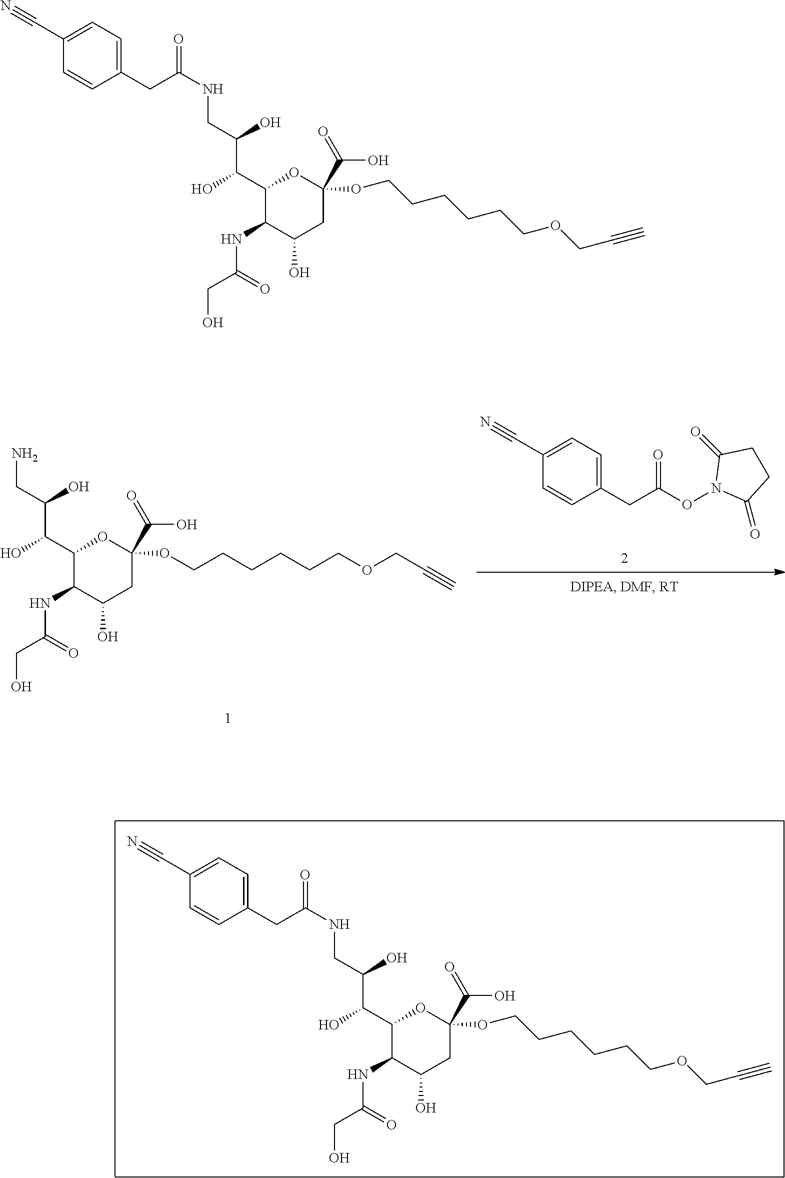 [SMILES: C#CCOCCCCCCO[C@]1(C(=O)O)C[C@H](O)[C@@H](NC(=O)CO)[C@H]([C@H](O)[C@H](O)CNC(=O)Cc2ccc(C#N)cc2)O1 ; C#CCOCCCCCCO[C@]1(C(=O)O)C[C@H](O)[C@@H](NC(=O)CO)[C@H]([C@H](O)[C@H](O)CN)O1 ; N#Cc1ccc(CC(=O)ON2C(=O)CCC2=O)cc1 ; C#CCOCCCCCCO[C@]1(C(=O)O)C[C@H](O)[C@@H](NC(=O)CO)[C@H]([C@H](O)[C@H](O)CNC(=O)Cc2ccc(C#N)cc2)O1]

### Synthesis of (2R,4S,5R,6R)-6-((1R,2R)-3-(2-(4-cyanophenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Compound 162)

To a stirred solution of (2R,4S,5R,6R)-6-((1R,2R)-3-amino-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (1, 0.30 g, 0.649 mmol) and 2,5-dioxopyrrolidin-1-yl 2-(4-cyanophenyl)acetate (2, 0.134 g, 0.519 mmol) in N,N-dimethylformamide (5.0 mL), was added DIPEA (0.565 mL, 3.24 mmol) at 0° C. and reaction mixture was stirred at room temperature for 4 h. After completion, the reaction was concentrated, and the crude residue was purified by prep HPLC using acetonitrile and water (+0.1% TFA) to afford the desired product. Yield: 0.030 g, 8%; LCMS m/z 606.40 [M+H]+; ¹H NMR (400 MHz, methanol-d₄): δ 7.68 (d, J=8.4 Hz, 2H), 7.50 (d, J=8.4 Hz, 2H), 4.12 (d, J=2.0 Hz, 2H), 4.01 (bs, 2H), 3.90-3.62 (m, 8H), 3.51 (t, J=6.8 Hz, 2H), 3.43-3.36 (m, 2H), 3.31-3.25 (m, 1H), 2.81 (t, J=2.4 Hz, 1H), 2.73 (dd, J=4.0 Hz & 12.4 Hz, 1H), 1.71 (t, J=12.4 Hz, 1H), 1.57-1.54 (m, 4H), 1.38-1.36 (m, 4H).

### (2R,4S,5R,6R)-6-((1R,2R)-3-(2-(4-chlorophenyl)acetamido)-1,2-dihydroxypropyl)-2-((2S,3S)-2,3-dihydroxy-4-(prop-2-yn-1-yloxy)butoxy)-4-hydroxy-5-(2-hydroxyacetamido)tetrahydro-2H-pyran-2-carboxylic acid (Compound 24)

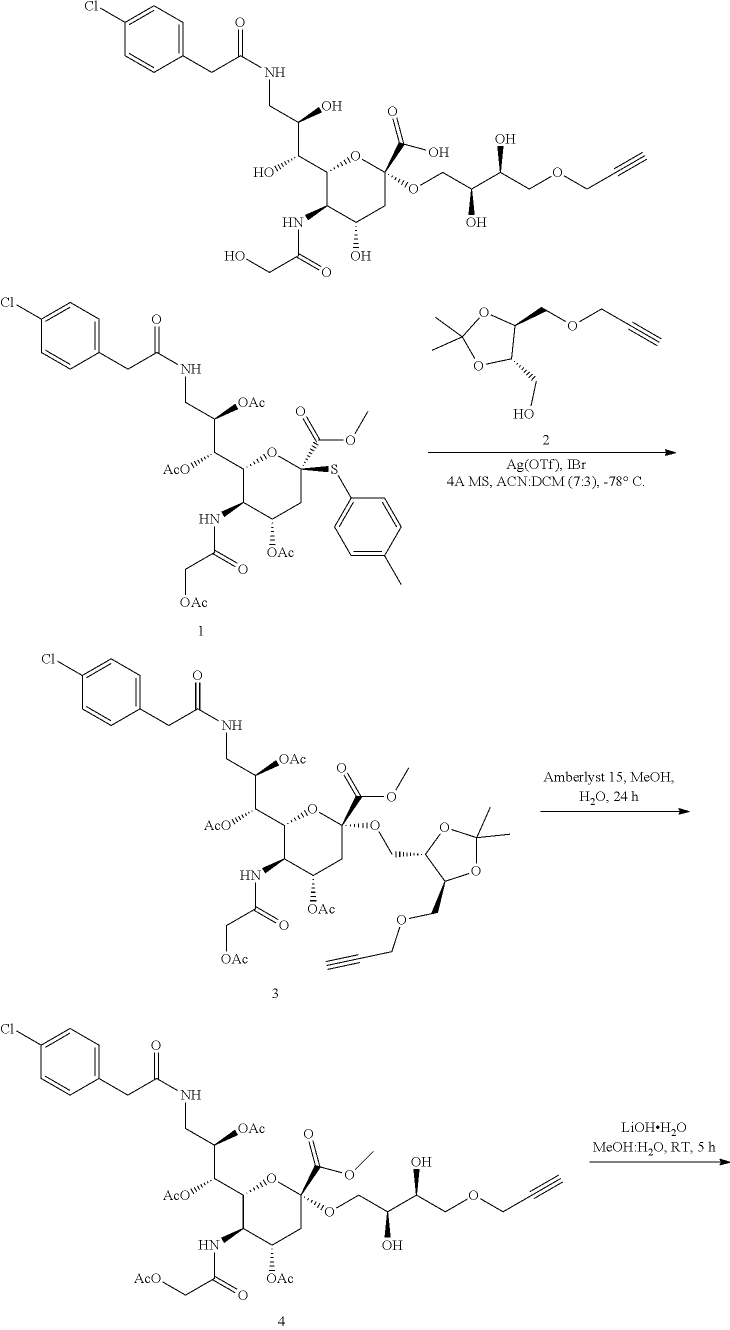

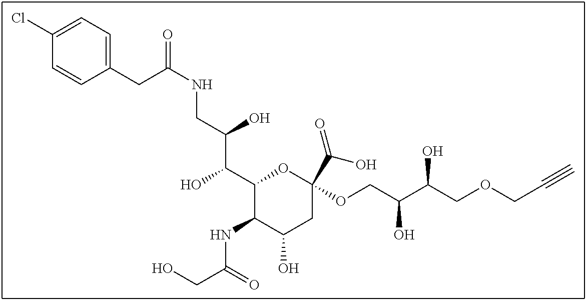 [SMILES: C#CCOC[C@H](O)[C@@H](O)CO[C@]1(C(=O)O)C[C@H](O)[C@@H](NC(=O)CO)[C@H]([C@H](O)[C@H](O)CNC(=O)Cc2ccc(Cl)cc2)O1]

### Synthesis of (1R,2R)-1-((2R,3R,4S,6R)-4-acetoxy-3-(2-acetoxyacetamido)-6-(((4S,5S)-2,2-dimethyl-5-((prop-2-yn-1-yloxy)methyl)-1,3-dioxolan-4-yl)methoxy)-6-(methoxycarbonyl)tetrahydro-2H-pyran-2-yl)-3-(2-(4-chlorophenyl)acetamido)propane-1,2-diyl diacetate (3)

To a solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (1, 0.30 g, 0.392 mmol) and ((4S,5S)-2,2-dimethyl-5-((prop-2-yn-1-yloxy)methyl)-1,3-dioxolan-4-yl)methanol (2, 0.392 g, 1.96 mmol) in anhydrous dichloromethane (10 mL) and anhydrous acetonitrile (12 mL), were added silver(I) trifluoromethanesulfonate (0.201 g, 0.784 mmol) and activated 4 Å powdered molecular sieves (1.00 g). The solution was stirred at room temperature for 1 h under nitrogen atmosphere then cooled to −78° C. Iodine monobromide (0.243 g, 1.18 mmol) in dichloromethane (1.0 mL) was added dropwise to the reaction mixture and was stirred at same temperature for 2 h. After completion the reaction mixture was quenched with triethylamine (1.0 mL) and warmed to room temperature. The reaction mixture was and the filtrate was washed by saturated solution of sodium bicarbonate and dried over sodium sulfate, filtered and concentrated. The crude product was purified by column chromatography using ethyl acetate and heptane as eluent to afford the desired product. Yield: 0.280 g, 84%; LCMS m/z 841.05 [M+H]+.

### Synthesis of (1R,2R)-1-((2R,3R,4S,6R)-4-acetoxy-3-(2-acetoxyacetamido)-6-((2S,3S)-2,3-dihydroxy-4-(prop-2-yn-1-yloxy)butoxy)-6-(methoxycarbonyl)tetrahydro-2H-pyran-2-yl)-3-(2-(4-chlorophenyl)acetamido)propane-1,2-diyl diacetate (4)

To a stirred solution of (1R,2R)-1-((2R,3R,4S,6R)-4-acetoxy-3-(2-acetoxyacetamido)-6-(((4S,5S)-2,2-dimethyl-5-((prop-2-yn-1-yloxy)methyl)-1,3-dioxolan-4-yl)methoxy)-6-(methoxycarbonyl)tetrahydro-2H-pyran-2-yl)-3-(2-(4-chlorophenyl)acetamido)propane-1,2-diyl diacetate (3, 0.280 g, 0.333 mmol) in methanol (5.0 mL) and water (1.0 mL), Amberlyst 15H⁺ (0.50 g) was added. The reaction mixture was stirred at 80° C. for 12 h. After completion the reaction mixture was filtered, washed with methanol and the filtrate was concentrated to afford the desired product which was used without further purification. Yield: 0.238 g, (crude); LCMS m/z 801.05 [M+H]+.

### Synthesis of (2R,4S,5R,6R)-6-((1R,2R)-3-(2-(4-chlorophenyl)acetamido)-1,2-dihydroxypropyl)-2-((2S,3S)-2,3-dihydroxy-4-(prop-2-yn-1-yloxy)butoxy)-4-hydroxy-5-(2-hydroxyacetamido)tetrahydro-2H-pyran-2-carboxylic acid

To a stirred solution of (1R,2R)-1-((2R,3R,4S,6R)-4-acetoxy-3-(2-acetoxyacetamido)-6-((2S,3S)-2,3-dihydroxy-4-(prop-2-yn-1-yloxy)butoxy)-6-(methoxycarbonyl)tetrahydro-2H-pyran-2-yl)-3-(2-(4-chlorophenyl)acetamido)propane-1,2-diyl diacetate (4, 0.238 g, 0.297 mmol) in methanol (5.0 mL), was added a solution of lithium hydroxide monohydrate (0.042 g, 1.78 mmol) in water (0.5 mL). The reaction mixture was stirred at room temperature for 5 h. After completion, the reaction mixture was treated with acidic resin (Dowex 50H⁺) to reach pH ˜6 and the suspension was filtered. The filtrate was concentrated, and the crude residue was purified by prep HPLC using acetonitrile and water (+0.1% TFA) to afford the desired product. Yield: 0.029 g, 16%; LCMS m/z 619.25 ^{[M+H]+}; ¹H NMR (400 MHz, methanol-d₄) 7.34-7.28 (m, 4H), 4.19 (m, 2H), 4.03 (bs, 2H), 3.95-3.73 (m, 7H), 3.68-3.59 (m, 4H), 3.55 (s, 2H), 3.36-3.33 (m, 1H), 3.30-3.24 (m, 1H), 2.86 (t, J=2.4 Hz, 1H), 2.75 (dd, J=3.6 Hz, 12.4 Hz, 1H), 1.81-1.75 (m, 1H).

### (2R,4S,5R,6R)-6-((1R,2R)-3-(2-(4-chlorophenyl)acetamido)-1,2-dihydroxypropyl)-4-hydroxy-5-(2-hydroxyacetamido)-2-((tetrahydro-2H-pyran-4-yl)methoxy)tetrahydro-2H-pyran-2-carboxylic acid (Compound 106)

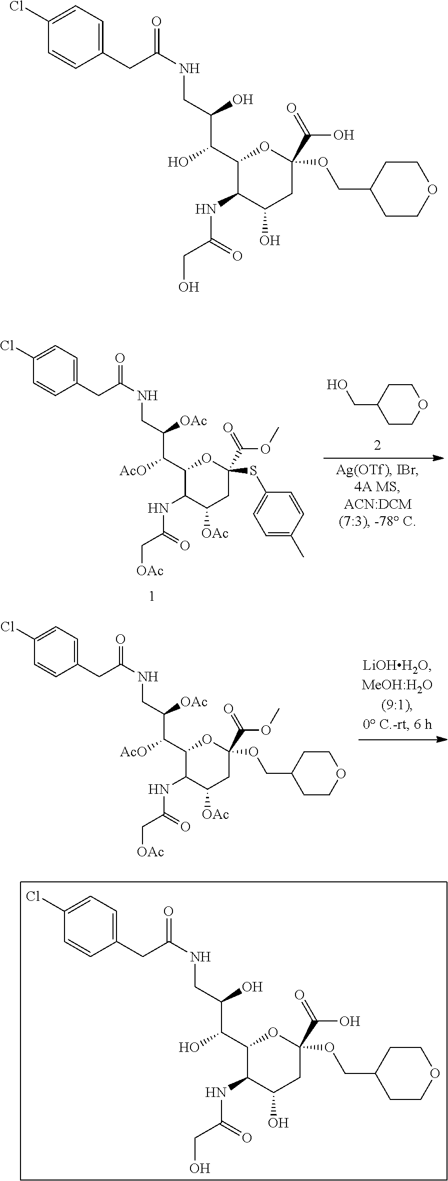

### Synthesis of (1R,2R)-1-((2R,3R,4S,6R)-4-acetoxy-3-(2-acetoxyacetamido)-6-(methoxycarbonyl)-6-((tetrahydro-2H-pyran-4-yl)methoxy)tetrahydro-2H-pyran-2-yl)-3-(2-(4-chlorophenyl)acetamido)propane-1,2-diyl diacetate (3)

To a solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (1, 0.70 g, 0.91 mmol) and (tetrahydro-2H-pyran-4-yl)methanol (2, 0.74 g, 6.4 mmol) in anhydrous dichloromethane (120 mL) and anhydrous acetonitrile (175 mL, 3.45 mol), were added silver(I) trifluoromethanesulfonate (0.71 g, 2.73 mmol) and activated 4 Å powdered molecular sieves (1.00 g). The solution was stirred at room temperature for 1 h under nitrogen atmosphere then cooled to −78° C. Iodine monobromide (0.38 g, 1.83 mmol) in dichloromethane (5.0 mL) was added dropwise to the reaction mixture and was stirred at same temperature for 2 h. After completion, the reaction mixture was quenched with triethylamine (2.0 mL) and warmed to room temperature. The reaction mixture was filtered and the filtrate was washed with a saturated solution of sodium bicarbonate and dried over sodium sulfate, filtered and concentrated. The crude product was purified by column chromatography using ethyl acetate and heptane to afford the desired product. Yield: 0.4 g, 18%; LCMS m/z 757.05 [M+H]+.

### Synthesis of (2R,4S,5R,6R)-6-((1R,2R)-3-(2-(4-chlorophenyl)acetamido)-1,2-dihydroxypropyl)-4-hydroxy-5-(2-hydroxyacetamido)-2-((tetrahydro-2H-pyran-4-yl)methoxy)tetrahydro-2H-pyran-2-carboxylic acid (Compound 106)

To a stirred solution of (1R,2R)-1-((2R,3R,4S,6R)-4-acetoxy-3-(2-acetoxyacetamido)-6-(methoxycarbonyl)-6-((tetrahydro-2H-pyran-4-yl)methoxy)tetrahydro-2H-pyran-2-yl)-3-(2-(4-chlorophenyl)acetamido)propane-1,2-diyl diacetate (3, 0.40 g, 0.53 mmol) in methanol (5 mL), was added a solution of lithium hydroxide monohydrate (0.075 g, 3.17 mmol) in water (0.5 mL). The reaction mixture was stirred at room temperature for 6 h. After completion, the reaction mixture was treated with acidic resin (Dowex 50, H⁺) to reach pH ˜6 and the suspension was filtered. The filtrate was concentrated and the crude residue was purified by prep HPLC using acetonitrile and water (+0.1% TFA) to afford the desired product. Yield: 0.138 g, 43%; LCMS m/z 575.35 [M+H]+; H NMR (400 MHz, Methanol-d₄) d ¹H NMR (400 MHz, Methanol-d₄) d 7.89 (d, J=7.6 Hz, 1H), 7.32-7.27 (m, 4H), 4.02 (s, 2H), 3.94-3.76 (m, 5H), 3.71-3.61 (m, 3H), 3.53 (s, 2H), 3.43-3.35 (m, 3H), 3.31-3.24 (m, 2H), 2.71 (dd, J=12.4 Hz & 4.0 Hz, 1H), 1.79-157 (m, 4H), 1.34-1.22 (m, 2H).

### (2R,4S,5R,6R)-2-((6-carboxyhexyl)oxy)-6-((1R,2R)-1,2-dihydroxy-3-(2-(3-hydroxyphenyl)acetamido)propyl)-4-hydroxy-5-(2-hydroxyacetamido)tetrahydro-2H-pyran-2-carboxylic acid (Compound 143)

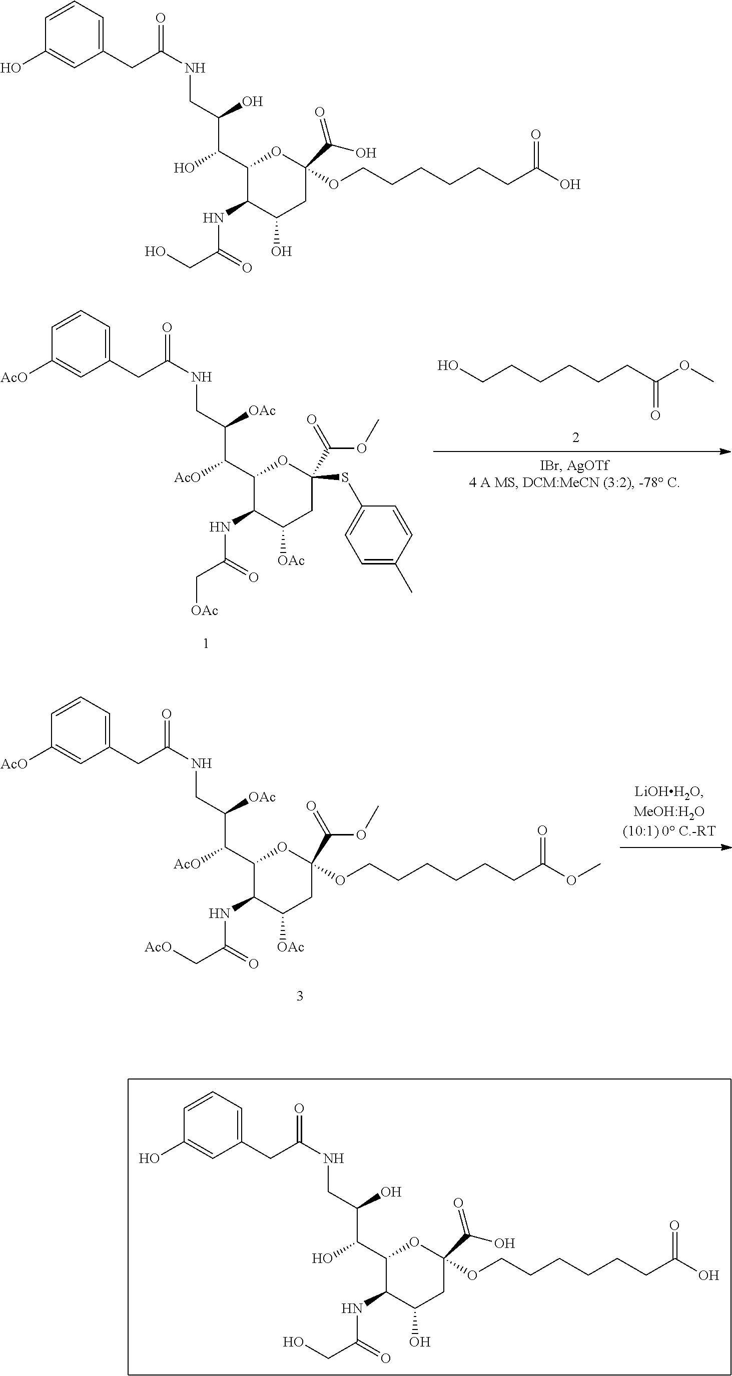 [SMILES: O=C(O)CCCCCCO[C@]1(C(=O)O)C[C@H](O)[C@@H](NC(=O)CO)[C@H]([C@H](O)[C@H](O)CNC(=O)Cc2cccc(O)c2)O1 ; COC(=O)[C@]1(Sc2ccc(C)cc2)C[C@H](OC(C)=O)[C@@H](NC(=O)COC(C)=O)[C@H]([C@H](OC(C)=O)[C@@H](CNC(=O)Cc2cccc(OC(C)=O)c2)OC(C)=O)O1 ; COC(=O)CCCCCCO ; COC(=O)CCCCCCO[C@]1(C(=O)OC)C[C@H](OC(C)=O)[C@@H](NC(=O)COC(C)=O)[C@H]([C@H](OC(C)=O)[C@@H](CNC(=O)Cc2cccc(OC(C)=O)c2)OC(C)=O)O1 ; O=C(O)CCCCCCO[C@]1(C(=O)O)C[C@H](O)[C@@H](NC(=O)CO)[C@H]([C@H](O)[C@H](O)CNC(=O)Cc2cccc(O)c2)O1]

### Synthesis of (1R,2R)-1-((2R,3R,4S,6R)-4-acetoxy-3-(2-acetoxyacetamido)-6-((7-methoxy-7-oxoheptyl)oxy)-6-(methoxycarbonyl)tetrahydro-2H-pyran-2-yl)-3-(2-(3-acetoxyphenyl)acetamido)propane-1,2-diyl diacetate (3)

To a solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(3-acetoxyphenyl)acetamido)propane-1,2-diyl diacetate (1, 0.50 g, 0.634 mmol) and methyl 7-hydroxyheptanoate (2, 0.508 g, 3.17 mmol) in anhydrous dichloromethane (16 mL) and anhydrous acetonitrile (25 mL), were added silver(I) trifluoromethanesulfonate (0.489 g, 1.90 mmol) and activated 4 Å powdered molecular sieves (1.00 g). The solution was stirred at room temperature for 1 h under nitrogen atmosphere then cooled to −78° C. Iodine monobromide (0.262 g, 1.27 mmol) in dichloromethane (5.0 mL) was added dropwise to the reaction mixture and was stirred at same temperature for 2 h. After completion the reaction mixture was quenched with triethylamine (2.0 mL) and warmed to room temperature. The reaction mixture was filtered, ssand the filtrate was washed with a saturated solution of sodium bicarbonate and dried over sodium sulfate, filtered and concentrated. The crude product was purified by column chromatography using ethyl acetate and heptane to afford the desired product. Yield: 0.40 g, 76%; LCMS m/z 825.15 [M+H]+.

### Synthesis of (2R,4S,5R,6R)-2-((6-carboxyhexyl)oxy)-6-((1R,2R)-1,2-dihydroxy-3-(2-(3-hydroxyphenyl)acetamido)propyl)-4-hydroxy-5-(2-hydroxyacetamido)tetrahydro-2H-pyran-2-carboxylic acid (Compound 143)

To a stirred solution of (1R,2R)-1-((2R,3R,4S,6R)-4-acetoxy-3-(2-acetoxyacetamido)-6-((7-methoxy-7-oxoheptyl)oxy)-6-(methoxycarbonyl)tetrahydro-2H-pyran-2-yl)-3-(2-(3-acetoxyphenyl)acetamido)propane-1,2-diyl diacetate (3, 0.40 g, 0.485 mmol) in methanol (10.0 mL) was added a solution of lithium hydroxide monohydrate (0.058 g, 2.42 mmol) in water (1.0 mL). The reaction mixture was stirred at room temperature for 4 h. After completion, the reaction mixture was treated with acidic resin (Dowex 50H⁺) to reach pH ˜6 and the suspension was filtered. The filtrate was concentrated, and the crude residue obtained was purified by prep HPLC using acetonitrile and water (+0.1% TFA) to afford the desired product. Yield: 0.075 g, 26%; LCMS m/z 587.40 [M+H]+; ¹H NMR (400 MHz, methanol-d₄): δ 7.88 (d, J=8.0 Hz, 1H), 7.11 (t, J=8.0 Hz, 1H), 6.76-6.73 (m, 2H), 6.66 (dd, J=8 Hz & 1.6 Hz, 1H), 4.01 (bs, 2H), 3.90-3.85 (m, 1H), 3.83-3.69 (m, 4H), 3.64 (dd, J=14 Hz & 3.2 Hz, 1H), 3.48 (bs, 2H), 3.43-3.36 (m, 2H), 3.31-3.24 (m, 1H), 2.71 (dd, J=12.8 Hz & 4.0 Hz, 1H), 2.28 (t, J=7.6 Hz, 2H), 1.72 (t, J=12.4 Hz, 1H), 1.61-1.53 (m, 4H), 1.36-1.35 (m, 4H).

### (2R,4S,5R,6R)-6-((1R,2R)-3-(4-chlorobenzamido)-1,2-dihydroxypropyl)-2-((2,3-difluorobenzyl)oxy)-5-(2-fluoroacetamido)-4-hydroxytetrahydro-2H-pyran-2-carboxylic acid (Compound 31)

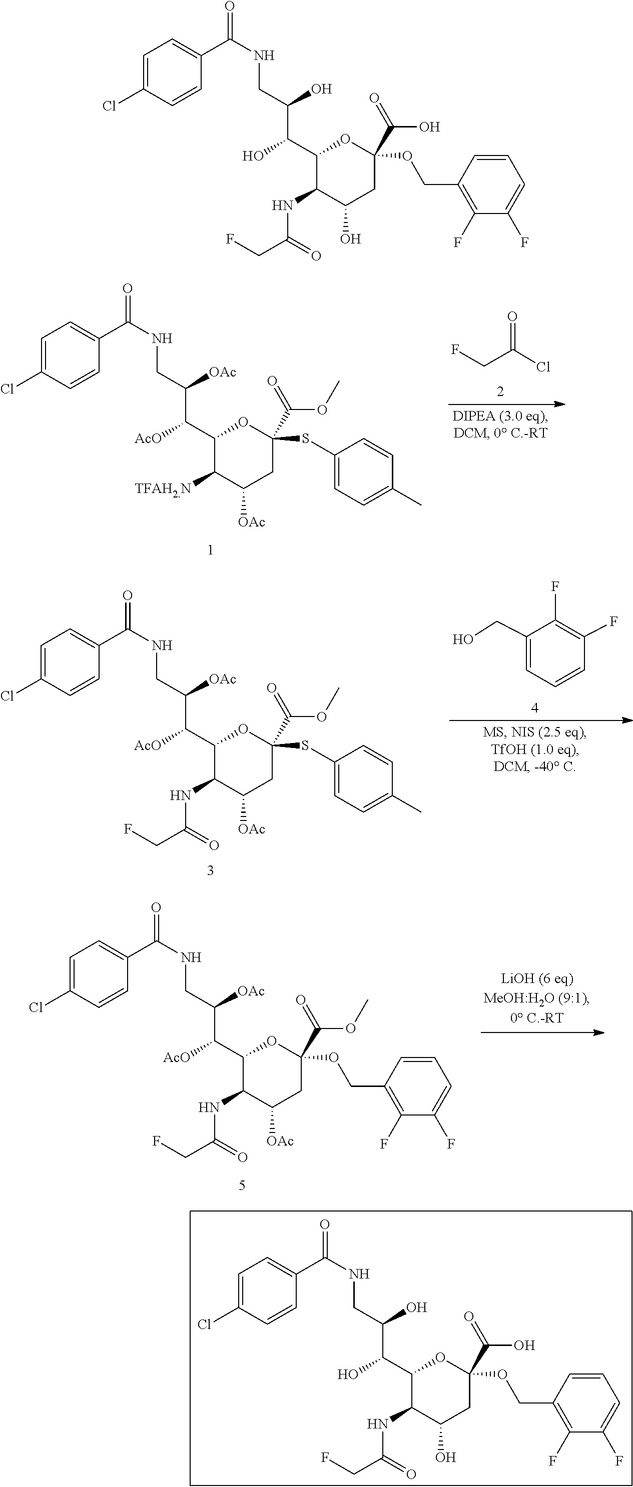

### Synthesis of (1R,2R)-1-((2R,3R,4S,6R)-4-acetoxy-3-(2-fluoroacetamido)-6-(methoxycarbonyl)-6-(p-tolylthio)tetrahydro-2H-pyran-2-yl)-3-(4-chlorobenzamido)propane-1,2-diyl diacetate (3)

To a stirred solution of (1R,2R)-1-((2R,3R,4S,6R)-4-acetoxy-6-(methoxycarbonyl)-6-(p-tolylthio)-3-((2,2,2-trifluoroacetyl)-14-azaneyl)tetrahydro-2H-pyran-2-yl)-3-(4-chlorobenzamido)propane-1,2-diyl diacetate (1, 0.50 g, 0.768 mmol) in dichloromethane (5.0 mL) was added ethylbis(propan-2-yl)amine (0.150 mL, 1.15 mmol) followed by drop wise addition of 2-fluoroacetyl chloride (2, 0.104 g, 0.921 mmol) at 0° C. The reaction mixture was stirred at 0° C. and progress of the reaction was monitored by TLC. After completion the reaction mixture was concentrated under reduced pressure to get crude which was purified by flash column chromatography using 70-75% ethyl acetate in hexane as eluent to afford (1R,2R)-1-((2R,3R,4S,6R)-4-acetoxy-3-(2-fluoroacetamido)-6-(methoxycarbonyl)-6-(p-tolylthio)tetrahydro-2H-pyran-2-yl)-3-(4-chlorobenzamido)propane-1,2-diyl diacetate (3) as a pale yellow solid. Yield: 0.30 g, 54.95%; LCMS (ESI) m/z 710.95 [M+H]+.

### Synthesis of (1R,2R)-1-((2R,3R,4S,6R)-4-acetoxy-6-((2,3-difluorobenzyl)oxy)-3-(2-fluoroacetamido)-6-(methoxycarbonyl)tetrahydro-2H-pyran-2-yl)-3-(4-chlorobenzamido)propane-1,2-diyl diacetate (5)

Solution of methyl (2S,4S,5R,6R)-4-(acetyloxy)-6-[(1R,2R)-1,2-bis(acetyloxy)-3-[(4-chlorophenyl)formamido]propyl]-5-(2-fluoroacetamido)-2-[(4-methylphenyl)sulfanyl]oxane-2-carboxylate (3, 0.180 g, 0.253 mmol), (2,3-difluorophenyl)methanol(4, 0.091 g, 0.633 mmol) and activated 4 Å powdered molecular sieves (250 mg) in anhydrous dichloromethane (5.0 mL) was stirred at room temperature for 15 hrs under nitrogen atmosphere. 1-iodopyrrolidine-2,5-dione (0.142 g, 0.633 mmol) and trifluoromethanesulfonic acid (0.038 g, 0.253 mmol) solution in dichloromethane (1.0 mL) was added at −40° C. and continued the reaction at same temperature for 1 hr. After completion, the reaction mixture was quenched by triethyl amine (1.0 mL) and warmed to room temperature. The reaction mixture was filtered through sintered funnel washed with dichloromethane. Filtrate was washed by saturated solution of sodium bicarbonate and dried over sodium sulfate, filtered and concentrated under reduced pressure to get crude compound. The crude product was purified by flash column chromatography using 30-40% ethyl acetate in hexane to afford methyl (2R,4S,5R,6R)-4-(acetyloxy)-6-[(1R,2R)-1,2-bis(acetyloxy)-3-[(4-chlorophenyl) formamido]propyl]-2-[(2,3-difluorophenyl)methoxy]-5-(2-fluoroacetamido)oxane-2-carboxylate (5) as a pale yellow solid. Yield: 0.1750 g, 94.5%; LCMS (ESI) m/z 730.95 [M+H]+.

### Synthesis of (2R,4S,5R,6R)-6-((1R,2R)-3-(4-chlorobenzamido)-1,2-dihydroxypropyl)-2-((2,3-difluorobenzyl)oxy)-5-(2-fluoroacetamido)-4-hydroxytetrahydro-2H-pyran-2-carboxylic acid (Compound 31)

To a stirred solution of methyl (2R,4S,5R,6R)-4-(acetyloxy)-6-[(1R,2R)-1,2-bis(acetyloxy)-3-[2-(4-chlorophenyl)acetamido]propyl]-2-[(2,3-difluorophenyl)methoxy]-5-(2-fluoroacetamido)oxane-2-carboxylate (5, 0.175 g, 0.235 mmol) in methanol (5.0 mL) was added solution of lithium hydroxide monohydrate (0.034 g, 1.41 mmol) in water (1.0 mL). The reaction mixture was stirred at room temperature for 6 hrs. The progress of the reaction was monitored by LCMS. After completion the reaction mixture was treated with acidic (Dowex 50H+) up to pH ˜6 and the suspension was filtered through sintered funnel. The filtrate was concentrated under reduced pressure to get crude which was purified by prep HPLC purification using acetonitrile in water with 0.1% TFA as buffer to afford (2R,4S,5R,6R)-6-((1R,2R)-3-(4-chlorobenzamido)-1,2-dihydroxypropyl)-2-((2,3-difluorobenzyl)oxy)-5-(2-fluoroacetamido)-4-hydroxytetrahydro-2H-pyran-2-carboxylic acid (Compound 31) as a white solid. Yield: 0.022 g, 15.5%; LCMS m/z 591.26 [M+H]+; ¹H NMR (400 MHz, methanol-d₄): δ 7.82 (d, J=8.4 Hz, 2H), 7.47 (d, J=8.4 Hz, 2H), 7.27-7.24 (m, 1H), 7.20-7.08 (m, 2H), 4.89-4.87 (m, 1H), 4.77 (s, 1H), 4.70 (d, J=12.0 Hz, 1H), 4.06-3.78 (m, 5H), 3.56-3.48 (m, 2H), 2.81-2.76 (dd, J=4.8 Hz, 12.8 Hz, 1H), 1.80 (t, J=11.6 Hz, 1H).

### (2S,4S,5R,6R)-2-((4-(but-3-yn-1-yloxy)benzyl)thio)-6-((1R,2R)-1,2-dihydroxy-3-(2-(3-hydroxyphenyl)acetamido)propyl)-4-hydroxy-5-(2-hydroxyacetamido)tetrahydro-2H-pyran-2-carboxylic acid (Compound 149)

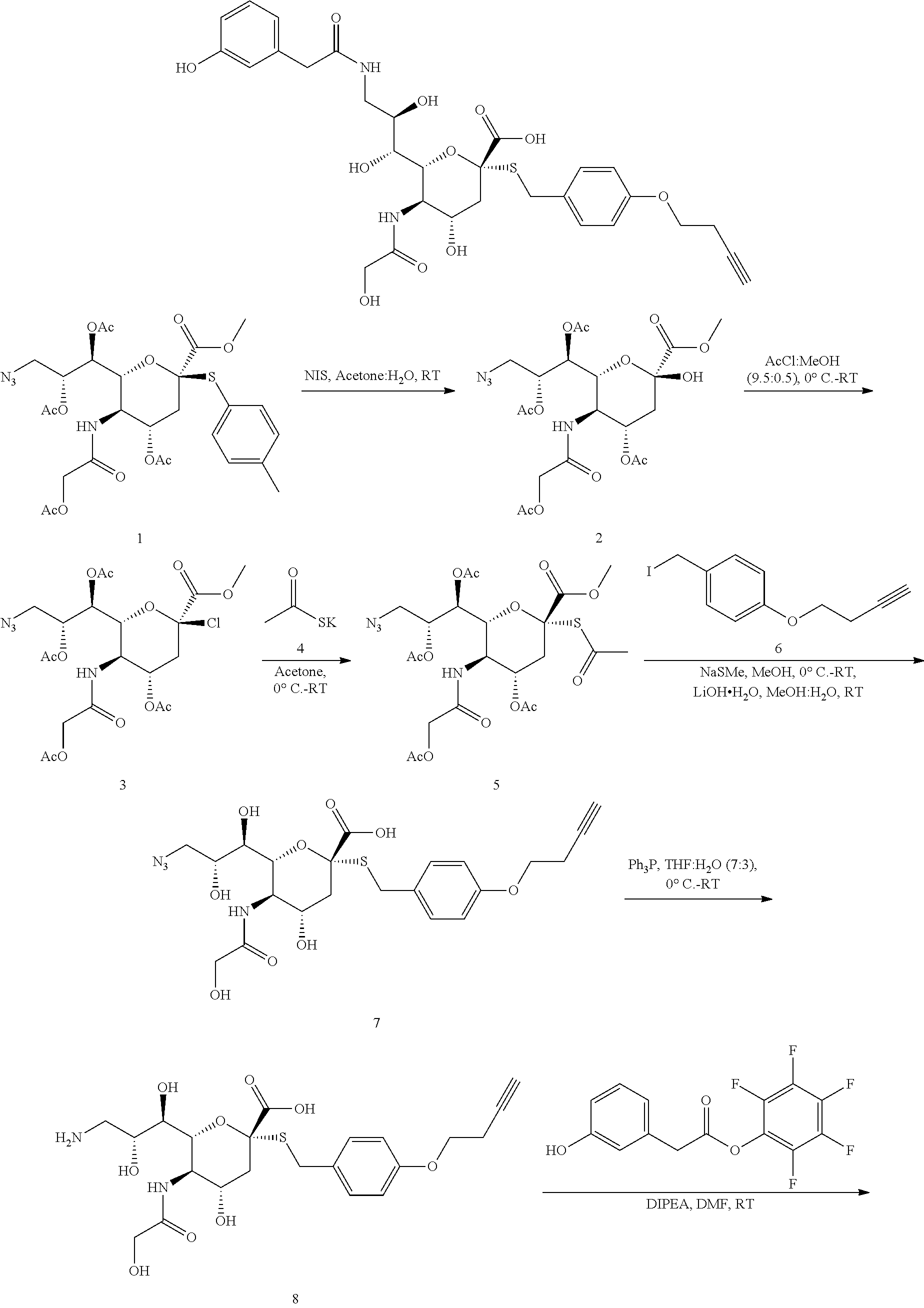

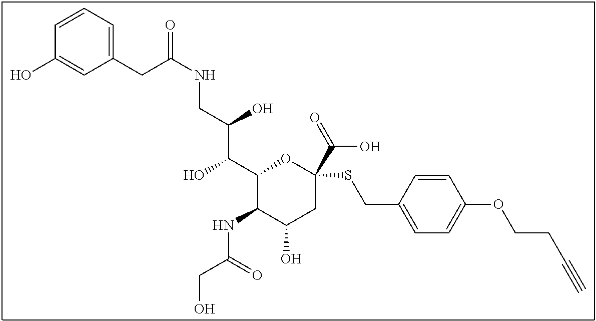 [SMILES: C#CCCOc1ccc(CS[C@]2(C(=O)O)C[C@H](O)[C@@H](NC(=O)CO)[C@H]([C@H](O)[C@H](O)CNC(=O)Cc3cccc(O)c3)O2)cc1]

### Synthesis of (1R,2R)-1-((2R,3R,4S,6S)-4-acetoxy-3-(2-acetoxyacetamido)-6-hydroxy-6-(methoxycarbonyl)tetrahydro-2H-pyran-2-yl)-3-azidopropane-1,2-diyl diacetate (2)

A stirred solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-azidopropane-1,2-diyl diacetate (1, 5.00 g, 7.830 mmol) in acetone: water (9:1, 50.0 mL) was added N-iodosuccinimide (6.63 g, 27.40 mmol) at 0° C. The reaction was maintained at 0° C. for 3 h. Reaction progress was monitored by TLC and after completion saturated aqueous solution of sodium metabisulfide (50.0 mL) and ethyl acetate (20.0 mL) was added to the reaction mixture. The reaction mixture was stirred for another 10 minutes and transferred to separatory funnel. Organic layer was separated and aqueous phase was extracted with ethyl acetate (10 mL). Organic layers were combined and washed sequentially with saturated sodium bicarbonate solution and water. Organic layer was dried over anhydrous sodium sulfate and concentrated under reduced pressure to obtain thick syrup. The thick syrup was purified with column chromatography using 60-75% ethyl acetate in heptane as eluent to afford (1R,2R)-1-((2R,3R,4S,6S)-4-acetoxy-3-(2-acetoxyacetamido)-6-hydroxy-6-(methoxycarbonyl)tetrahydro-2H-pyran-2-yl)-3-azidopropane-1,2-diyl diacetate (2) as white solid. Yield: 3.00 g, 72%; LCMS (ESI) m/z 533.00 [M+H]+.

### Synthesis of (1R,2R)-1-((2R,3R,4S,6R)-4-acetoxy-3-(2-acetoxyacetamido)-6-chloro-6-(methoxycarbonyl)tetrahydro-2H-pyran-2-yl)-3-azidopropane-1,2-diyl diacetate (3)

To a stirred solution of (1R,2R)-1-((2R,3R,4S,6S)-4-acetoxy-3-(2-acetoxyacetamido)-6-hydroxy-6-(methoxycarbonyl)tetrahydro-2H-pyran-2-yl)-3-azidopropane-1,2-diyl diacetate (2, 3.00 g, 5.63 mmol) in acetyl chloride (150.0 mL), was added anhydrous methanol (6.0 mL) dropwise at 0° C. The resulting reaction mixture was stirred at room temperature for 24 h and progress of the reaction was monitored by TLC. After completion the reaction mixture was concentrated under reduced pressure to get crude (1R,2R)-1-((2R,3R,4S,6R)-4-acetoxy-3-(2-acetoxyacetamido)-6-chloro-6-(methoxycarbonyl)tetrahydro-2H-pyran-2-yl)-3-azidopropane-1,2-diyl diacetate (3) as light brown gel which was used as such for the next step. Yield: 3.0 g, (crude).

### Synthesis of (1R,2R)-1-((2R,3R,4S,6S)-4-acetoxy-3-(2-acetoxyacetamido)-6-(acetylthio)-6-(methoxycarbonyl)tetrahydro-2H-pyran-2-yl)-3-azidopropane-1,2-diyl diacetate (5)

In an inert atmosphere crude (1R,2R)-1-((2R,3R,4S,6R)-4-acetoxy-3-(2-acetoxyacetamido)-6-chloro-6-(methoxycarbonyl)tetrahydro-2H-pyran-2-yl)-3-azidopropane-1,2-diyl diacetate (3, 3.0 g, 5.45 mmol, crude) was dissolved under stirring in dry acetone (30.0 mL) and cooled at 0° C. To this solution, potassium thioacetate (4, 1.870 g, 16.30 mmol) was added lot wise at 0° C.; the reaction mixture was stirred for 3 h at 0° C. and progress was monitored by TLC. After completion the reaction mixture was concentrated under reduced pressure to get crude. The crude was dissolved in ethyl acetate and resulting solution was washed with 1N HCl followed by water. Organic layer was separated, dried over anhydrous sodium sulfate and concentrated under reduced pressure to get thick residue. This was purified by column chromatography using in 60-70% ethyl acetate in heptane as eluent to get (1R,2R)-1-((2R,3R,4S,6S)-4-acetoxy-3-(2-acetoxyacetamido)-6-(acetylthio)-6-(methoxycarbonyl)tetrahydro-2H-pyran-2-yl)-3-azidopropane-1,2-diyl diacetate (5) as a white solid. Yield: 2.00 g, 62%; LCMS (ESI) m/z 590.90 [M+H]+.

### Synthesis of (2S,4S,5R,6R)-6-((1R,2R)-3-azido-1,2-dihydroxypropyl)-2-((4-(but-3-yn-1-yloxy)benzyl)thio)-4-hydroxy-5-(2-hydroxyacetamido)tetrahydro-2H-pyran-2-carboxylic acid (7)

To a stirred solution of (1R,2R)-1-((2R,3R,4S,6S)-4-acetoxy-3-(2-acetoxyacetamido)-6-(acetylthio)-6-(methoxycarbonyl)tetrahydro-2H-pyran-2-yl)-3-azidopropane-1,2-diyl diacetate (6, 0.50 g, 0.847 mmol) in methanol (5.0 mL) was added sodium thiomethoxide (0.086 g, 0.1.02 mmol) at 0° C. and stirred for 1 h. To the reaction, 1-(but-3-yn-1-yloxy)-4-(iodomethyl)benzene (6, 0.484 g, 1.69 mmol) was added at 0° C. and reaction mixture was stirred at room temperature for 1 h. Reaction was monitored by LCMS. After completion, to the same reaction mass was added lithium hydroxide (0.213 g, 5.08 mmol) at room temperature and the reaction was stirred at room temperature for 6 h. The progress of reaction was monitored by LCMS and after completion, Dowex-hydrogen form was added to pH˜6 and reaction mass filtered through sintered funnel. The filtrate was concentrated under reduced pressure to obtain thick residue. This was purified by trituration with diethyl ether to afford (2S,4S,5R,6R)-6-((1R,2R)-3-azido-1,2-dihydroxypropyl)-2-((4-(but-3-yn-1-yloxy)benzyl)thio)-4-hydroxy-5-(2-hydroxyacetamido)tetrahydro-2H-pyran-2-carboxylic acid (7) as a white solid. Yield: 0.30 g, 68%; LCMS (ESI) m/z 525.20 [M+H]+.

### Synthesis of (2S,4S,5R,6R)-6-((1R,2R)-3-amino-1,2-dihydroxypropyl)-2-((4-(but-3-yn-1-yloxy)benzyl)thio)-4-hydroxy-5-(2-hydroxyacetamido)tetrahydro-2H-pyran-2-carboxylic acid (8)

To a stirred solution of (2S,4S,5R,6R)-6-((1R,2R)-3-azido-1,2-dihydroxypropyl)-2-((4-(but-3-yn-1-yloxy)benzyl)thio)-4-hydroxy-5-(2-hydroxyacetamido)tetrahydro-2H-pyran-2-carboxylic acid (7, 0.60 g, 1.14 mmol) in tetrahydrofuran (7.0 mL) and water (3.0 mL) was added triphenylphosphane (0.870 g, 11.40 mmol) at 0° C. Reaction mixture was allowed to stirred at room temperature for 12 h. Progress of the reaction was monitored by LCMS. After completion reaction mixture was concentrated under reduced pressure to get crude. Crude was dissolved in water and ethyl acetate. Aqueous layer was separated and lyophilized to afford (2S,4S,5R,6R)-6-((1R,2R)-3-amino-1,2-dihydroxypropyl)-2-((4-(but-3-yn-1-yloxy)benzyl)thio)-4-hydroxy-5-(2-hydroxyacetamido)tetrahydro-2H-pyran-2-carboxylic acid (8) as off white solid. Yield: 0.50 g, 87%; LCMS m/z 499.12 [M+H]+.

### Synthesis of (2S,4S,5R,6R)-2-((4-(but-3-yn-1-yloxy)benzyl)thio)-6-((1R,2R)-1,2-dihydroxy-3-(2-(3-hydroxyphenyl)acetamido)propyl)-4-hydroxy-5-(2-hydroxyacetamido)tetrahydro-2H-pyran-2-carboxylic acid (Compound 149)

To a stirred solution of (2S,4S,5R,6R)-6-((1R,2R)-3-amino-1,2-dihydroxypropyl)-2-((4-(but-3-yn-1-yloxy)benzyl)thio)-4-hydroxy-5-(2-hydroxyacetamido)tetrahydro-2H-pyran-2-carboxylic acid (8, 0.20 g, 0.401 mmol) and perfluorophenyl 2-(3-hydroxyphenyl)acetate (9, 0.102 g, 0.320 mmol) in dimethylformamide (3.0 mL) was added ethylbis(propan-2-yl)amine (0.34 mL, 2.01 mmol) at 0° C. and reaction mixture was stirred at room temperature for 4 h. Progress of the reaction was monitored by LCMS. After completion the reaction mixture was concentrated under reduced pressure to get crude. The crude material was purified by prep HPLC purification using 0.1% TFA in water/Acetonitrile to afford (2S,4S,5R,6R)-2-((4-(but-3-yn-1-yloxy)benzyl)thio)-6-((1R,2R)-1,2-dihydroxy-3-(2-(3-hydroxyphenyl)acetamido)propyl)-4-hydroxy-5-(2-hydroxyacetamido)tetrahydro-2H-pyran-2-carboxylic acid (Compound 149) as off white solid. Yield: 0.045 g, 18%; LCMS m/z 633.45 [M+H]+. 1H NMR (400 MHz, methanol-d4): δ 7.95 (d, J=8.4 Hz, 1H), 7.22 (d, J=8.8 Hz, 2H), 7.06 (t, J=8.0 Hz, 1H), 6.87-6.83 (m, 2H), 6.74-6.72 (m, 2H), 6.63-6.61 (m, 1H), 4.06 (t, J=6.8 Hz, 2H), 4.02 (s, 2H), 3.98 (d, J=12 Hz, 1H), 3.91-3.78 (m, 4H), 3.65-3.60 (m, 2H), 3.46 (s, 2H), 3.37 (dd, J=8.8 Hz & 1.6 Hz, 1H), 3.26-3.21 (m, 1H), 2.78 (dd, J=12.8 Hz & 4.0 Hz, 1H), 2.63 (dt, J=6.8 Hz & 2.8 Hz, 2H), 2.32 (t, J=2.8 Hz, 1H), 1.81-1.75 (m, 1H).

### (2R,4S,5R,6R)-2-((4-(but-3-yn-1-yloxy)benzyl)oxy)-6-((1R,2R)-1,2-dihydroxy-3-(2-(4-morpholinophenyl)acetamido)propyl)-4-hydroxy-5-(2-hydroxyacetamido)tetrahydro-2H-pyran-2-carboxylic acid (Compound 139)

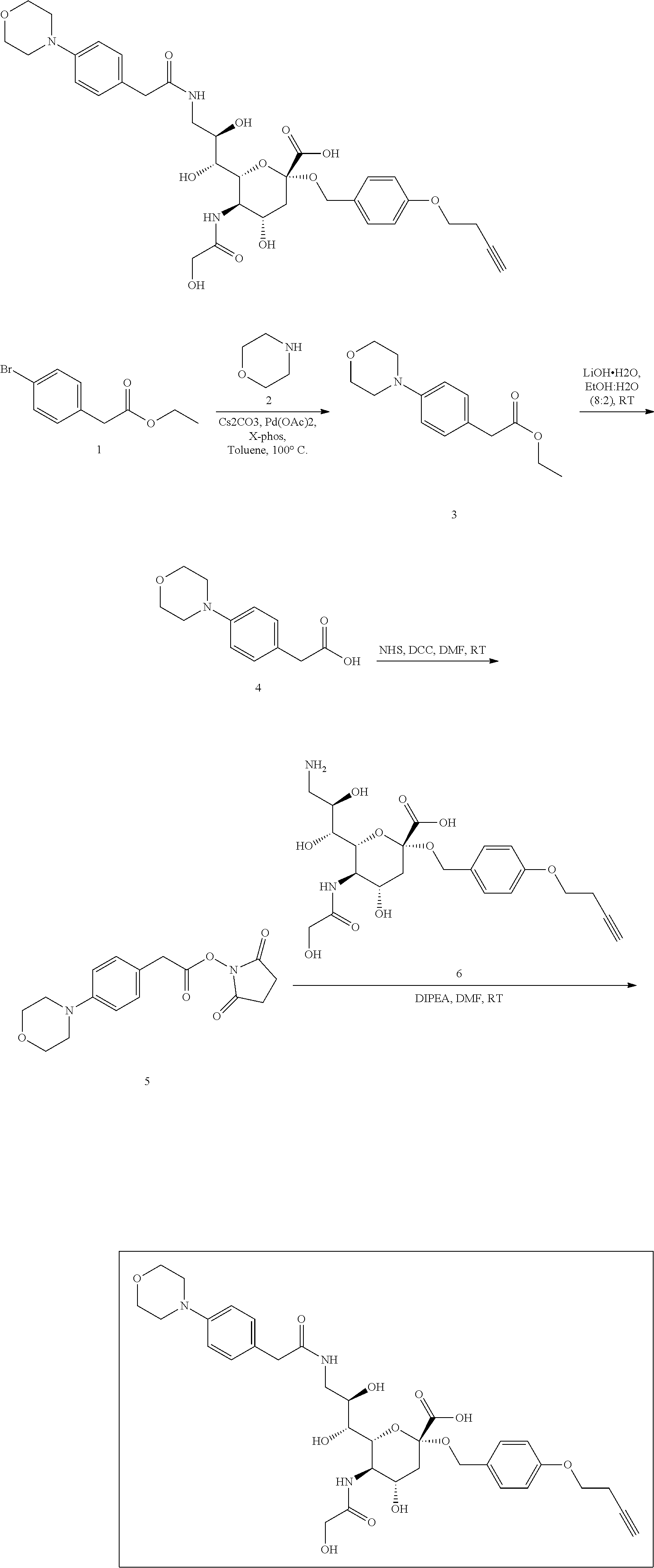

### Synthesis of ethyl 2-(4-morpholinophenyl)acetate (3)

To a stirred solution of ethyl 2-(4-bromophenyl)acetate (1, 1.00 g, 4.11 mmol) in degassed toluene (10.0 mL) were added cesium carbonate (2.01 g, 6.17 mmol), dicyclohexyl[2′,4′,6′-tris(propan-2-yl)-[1,1′-biphenyl]-2-yl]phosphane (0.196 g, 0.411 mmol) and palladium(2+) diacetate (0.046 g, 0.206 mmol) followed by morpholine (2, 0.71 g, 8.23 mmol) at room temperature. The reaction mixture was stirred at 1000° C. for 12 h. Progress of the reaction was monitored by TLC and LCMS. After completion the reaction mixture was diluted with water and ethyl acetate. Organic layer was separated, dried over sodium sulphate, filtered and concentrated under reduced pressure to get crude. The crude was purified by column chromatography using 20-25% ethyl acetate in heptane as eluent to afford ethyl 2-(4-morpholinophenyl)acetate (3) as yellow solid. Yield: 0.60 g, 59%; LCMS (ESI) m/z 250.15 [M+H]+.

### Synthesis of 2-(4-morpholinophenyl)acetic acid (4)

To a stirred solution of ethyl 2-(4-morpholinophenyl)acetate (3, 0.50 g, 2.01 mmol) in ethanol (5.0 mL) was added solution of lithium hydroxide monohydrate (0.096 g, 4.01 mmol) in water (2.0 mL). The reaction mixture was stirred at room temperature for 5 h. Progress of the reaction was monitored by TLC and LCMS. After completion the reaction mixture was concentrated under reduced pressure and the mixture was treated with 1N HCl solution and the suspension was filtered through sintered funnel and washed with water and diethyl ether to afford 2-(4-morpholinophenyl)acetic acid (4) as off white solid. Yield: 0.250 g, 56%; LCMS (ESI) m/z 222.15 [M+H]+.

### Synthesis of 2,5-dioxopyrrolidin-1-yl 2-(4-morpholinophenyl)acetate (5)

To a stirred solution of 2-(4-morpholinophenyl)acetic acid (4, 0.170 g, 0.768 mmol) in N, N-dimethylformamide (2.0 mL), 1-hydroxypyrrolidine-2,5-dione (0.323 g, 2.80 mmol) and N,N′-dicyclohexylmethanediimine (0.159 g, 0.768 mmol) were added at 0° C. and reaction mixture was stirred at room temperature for 4 h. Progress of the reaction was monitored by TLC and LCMS. After completion the reaction mixture was concentrated under reduced pressure. The reaction mixture was dissolved in ethyl acetate and filtered through sintered funnel and washed with ethyl acetate to afford 2,5-dioxopyrrolidin-1-yl 2-(4-morpholinophenyl)acetate (5) as off white solid. Yield: 0.120 g, 49%; LCMS (ESI) m/z 319.05 [M+H]+.

### Synthesis of (2R,4S,5R,6R)-2-((4-(but-3-yn-1-yloxy)benzyl)oxy)-6-((1R,2R)-1,2-dihydroxy-3-(2-(4-morpholinophenyl)acetamido)propyl)-4-hydroxy-5-(2-hydroxyacetamido)tetrahydro-2H-pyran-2-carboxylic acid (Compound 139)

To a stirred solution of (2R,4S,5R,6R)-6-((1R,2R)-3-amino-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (6, 0.20 g, 0.414 mmol) and 2,5-dioxopyrrolidin-1-yl 2-(4-morpholinophenyl)acetate (5, 0.105 g, 0.331 mmol) in dimethylformamide (2.0 mL) was added ethylbis(propan-2-yl)amine (0.36 mL, 2.07 mmol) at 0° C. and reaction mixture was stirred at room temperature for 4 h. Progress of the reaction was monitored by LCMS. After completion the reaction mixture was concentrated under reduced pressure to get crude. The crude material was purified by prep HPLC using 0.1% TFA in water/Acetonitrile to afford (2R,4S,5R,6R)-2-((4-(but-3-yn-1-yloxy)benzyl)oxy)-6-((1R,2R)-1,2-dihydroxy-3-(2-(4-morpholinophenyl)acetamido)propyl)-4-hydroxy-5-(2-hydroxyacetamido)tetrahydro-2H-pyran-2-carboxylic acid (Compound 139) as off white solid. Yield: 0.048 g, 17%; LCMS m/z 686.50 [M+H]+. 1H NMR (400 MHz, methanol-d4): δ 7.27 (d, J=8.8 Hz, 2H), 7.18 (d, J=8.8 Hz, 2H), 6.87 (d, J=8.8 Hz, 4H), 4.72 (d, J=10.8 Hz, 1H), 4.43 (d, J=10.8 Hz, 1H), 4.07 (t, J=6.8 Hz, 2H), 4.02 (s, 2H), 3.93 (dt, J=2.8 Hz and 8.4 Hz, 1H), 3.85-3.71 (m, 8H), 3.45 (d, J=3.2 Hz, 2H), 3.37 (d, J=10.4 Hz, 1H), 3.19-3.14 (m, 1H), 3.04-3.01 (m, 4H), 2.87 (dd, J=4.0 Hz and 12.0 Hz, 1H), 2.64 (dt, J=2.8 Hz and 6.8 Hz, 2H), 2.32 (t, J=2.6 Hz, 1H), 1.63 (t, J=11.6 Hz, 1H).

### (2R,4S,5R,6R)-2-((4-(but-3-yn-1-yloxy)benzyl)oxy)-6-((1R,2R)-1,2-dihydroxy-3-(2-(3′-hydroxy-[1,1′-biphenyl]-3-yl)acetamido)propyl)-4-hydroxy-5-(2-hydroxyacetamido)tetrahydro-2H-pyran-2-carboxylic acid (Compound 45)

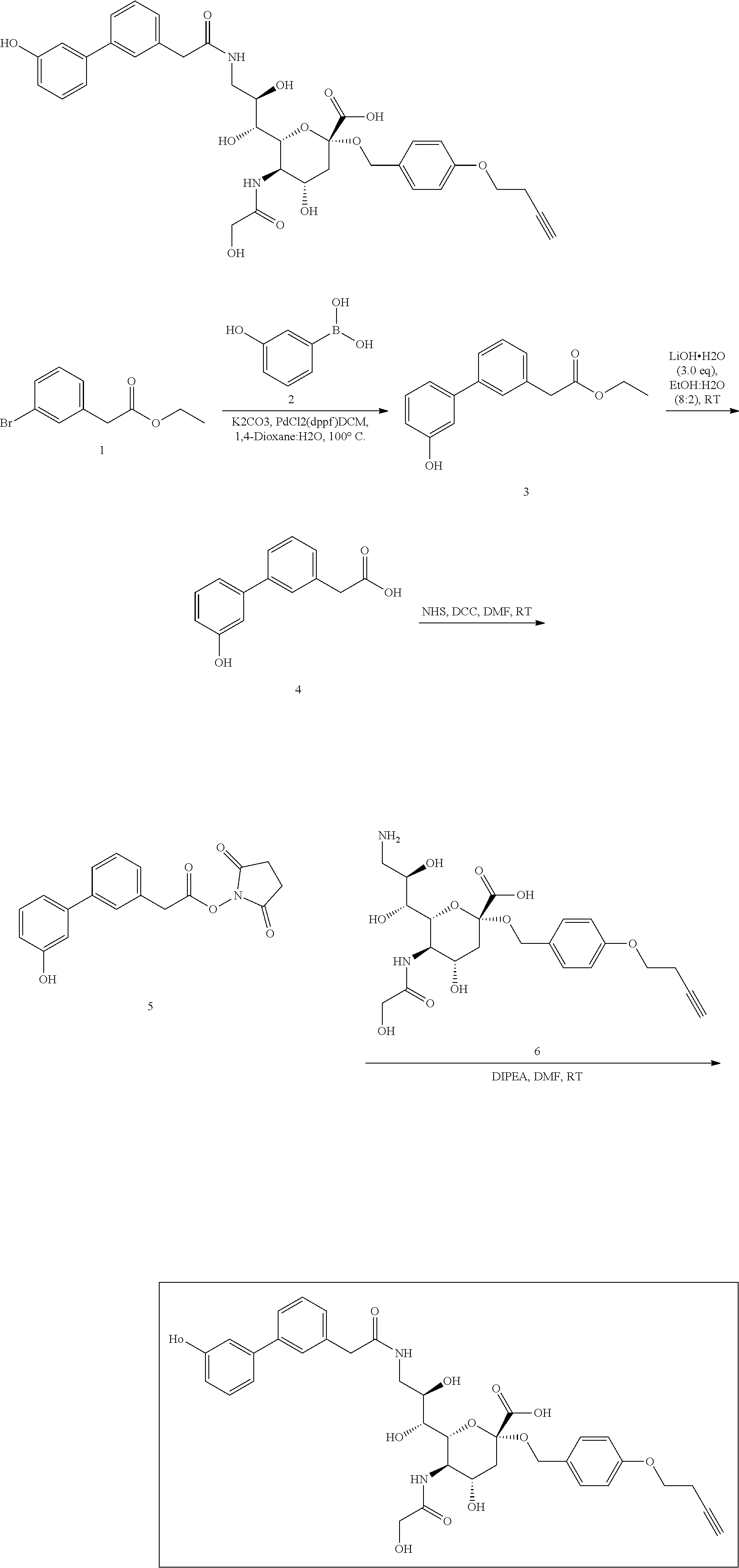

### Synthesis of ethyl 2-(3′-hydroxy-[1,1′-biphenyl]-3-yl)acetate (3)

To a stirred solution of ethyl 2-(3-bromophenyl)acetate (1, 1.00 g, 4.11 mmol) and (3-hydroxyphenyl)boronic acid (2, 0.851 g, 6.170 mmol) in 1,4-dioxane (8.0 mL) and water (2.0 mL) was added potassium carbonate (1.71 g, 12.3 mmol). Reaction mixture was degassed for 10 minutes with nitrogen bubbling and then [1,1′-Bis(diphenylphosphino)ferrocene]dichloropalladium(II) (0.168 g, 0.206 mmol) was added. The reaction mixture was allowed to stirred at 1000° C. for 12 h. Progress of the reaction was monitored by TLC and LCMS. After completion the reaction mixture was diluted with water and ethyl acetate. Organic layer was separated, dried over sodium sulphate, filtered and concentrated under reduced pressure to get crude. The crude was purified by column chromatography and desired compound was eluted with 15-20% ethyl acetate in heptane to afford ethyl 2-(3′-hydroxy-[1,1′-biphenyl]-3-yl)acetate (3) as off white solid. Yield: 0.80 g, 76%; LCMS (ESI) m/z 255.05 [M−1]⁻.

### Synthesis of 2-(3′-hydroxy-[1,1′-biphenyl]-3-yl)acetic acid (4)

To a stirred solution of ethyl 2-(3′-hydroxy-[1,1′-biphenyl]-3-yl)acetate (3, 1.0 g, 3.90 mmol) in ethanol (8.0 mL) was added solution of lithium hydroxide monohydrate (0.491 g, 11.70 mmol) in water (2.0 mL). The reaction mixture was stirred at room temperature for 5 h. Progress of the reaction was monitored by TLC and LCMS. After completion the reaction mixture was concentrated under reduced pressure and the mixture was treated with 1N HCl solution and the suspension was filtered through sintered funnel and washed with water and diethyl ether to afford 2-(3′-hydroxy-[1,1′-biphenyl]-3-yl)acetic acid (3) as off white solid. Yield: 0.60 g, 67%; LCMS (ESI) m/z 227.00 [M−1]⁻.

### Synthesis of 2,5-dioxopyrrolidin-1-yl 2-(3′-hydroxy-[1,1′-biphenyl]-3-yl)acetate (5)

To a stirred solution of 2-(3′-hydroxy-[1,1′-biphenyl]-3-yl)acetic acid (4, 0.370 g, 1.62 mmol) in N, N-dimethylformamide (4.0 mL) 1-hydroxypyrrolidine-2,5-dione (0.187 g, 1.62 mmol) and N,N′-dicyclohexylmethanediimine (0.334 g, 1.62 mmol) was added at 0° C. and reaction mixture was stirred at room temperature for 4 h. Progress of the reaction was monitored by TLC and LCMS. After completion the reaction mixture was concentrated under reduced pressure and residue was dissolved in ethyl acetate and filtered through sintered funnel and washed with ethyl acetate to afford 2,5-dioxopyrrolidin-1-yl 2-(3′-hydroxy-[1,1′-biphenyl]-3-yl)acetate (5) as off white solid. Yield: 0.300 g, 59%; LCMS (ESI) m/z 323.95 [M−1]⁻.

### Synthesis of (2R,4S,5R,6R)-2-((4-(but-3-yn-1-yloxy)benzyl)oxy)-6-((1R,2R)-1,2-dihydroxy-3-(2-(3′-hydroxy-[1,1′-biphenyl]-3-yl)acetamido)propyl)-4-hydroxy-5-(2-hydroxyacetamido)tetrahydro-2H-pyran-2-carboxylic acid (Compound 45)

To a stirred solution of (2R,4S,5R,6R)-6-((1R,2R)-3-amino-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (6, 0.20 g, 0.414 mmol) and 2,5-dioxopyrrolidin-1-yl 2-(3′-hydroxy-[1,1′-biphenyl]-3-yl)acetate (5, 0.107 g, 0.331 mmol) in dimethylformamide (2.0 mL) was added ethylbis(propan-2-yl)amine (0.36 mL, 2.07 mmol) at 0° C. and reaction mixture was stirred at room temperature for 4 h. Progress of the reaction was monitored by LCMS. After completion the reaction mixture was concentrated under reduced pressure to get crude. The crude material was purified by prep HPLC using 0.1% TFA in water/Acetonitrile to afford (2R,4S,5R,6R)-2-((4-(but-3-yn-1-yloxy)benzyl)oxy)-6-((1R,2R)-1,2-dihydroxy-3-(2-(3′-hydroxy-[1,1′-biphenyl]-3-yl)acetamido)propyl)-4-hydroxy-5-(2-hydroxyacetamido)tetrahydro-2H-pyran-2-carboxylic acid (Compound 45) as off white solid. Yield: 0.064 g, 22%; LCMS m/z 693.4 [M+H]+. 1H NMR (400 MHz, methanol-d4): δ 7.87 (d, J=7.6 Hz, 1H), 7.54-7.53 (m, 1H), 7.44-7.41 (m, 1H), 7.34 (t, J=7.6 Hz, 1H), 7.28-7.20 (m, 4H), 7.08-7.05 (m, 1H), 7.02 (t, J=2.0 Hz, 1H), 6.87-6.84 (m, 2H), 6.76-6.74 (m, 1H), 4.71 (d, J=10.8 Hz, 1H), 4.39 (d, J=10.8 Hz, 1H), 4.05 (t, J=6.8 Hz, 2H), 4.01 (s, 2H), 3.97-3.90 (m, 1H), 3.88-3.76 (m, 3H), 3.60 (dd, J=3.2 Hz and 13.6 Hz, 1H), 3.6 (bs, 2H), 3.41 (dd, J=1.6 Hz and 8.8 Hz, 1H), 3.30-3.25 (m, 1H), 2.78 (dd, J=4.0 Hz and 13.6 Hz, 1H), 2.63 (dt, J=6.8 Hz and 2.8 Hz, 2H), 2.32 (t, J=2.8 Hz, 1H), 1.81-1.75 (m, 1H).

### (2R,4S,5R,6R)-2-((4-(but-3-yn-1-yloxy)benzyl)oxy)-6-((1R,2R)-1,2-dihydroxy-3-(2-(4′-hydroxy-[1,1′-biphenyl]-3-yl)acetamido)propyl)-4-hydroxy-5-(2-hydroxyacetamido)tetrahydro-2H-pyran-2-carboxylic acid (Compound 44)

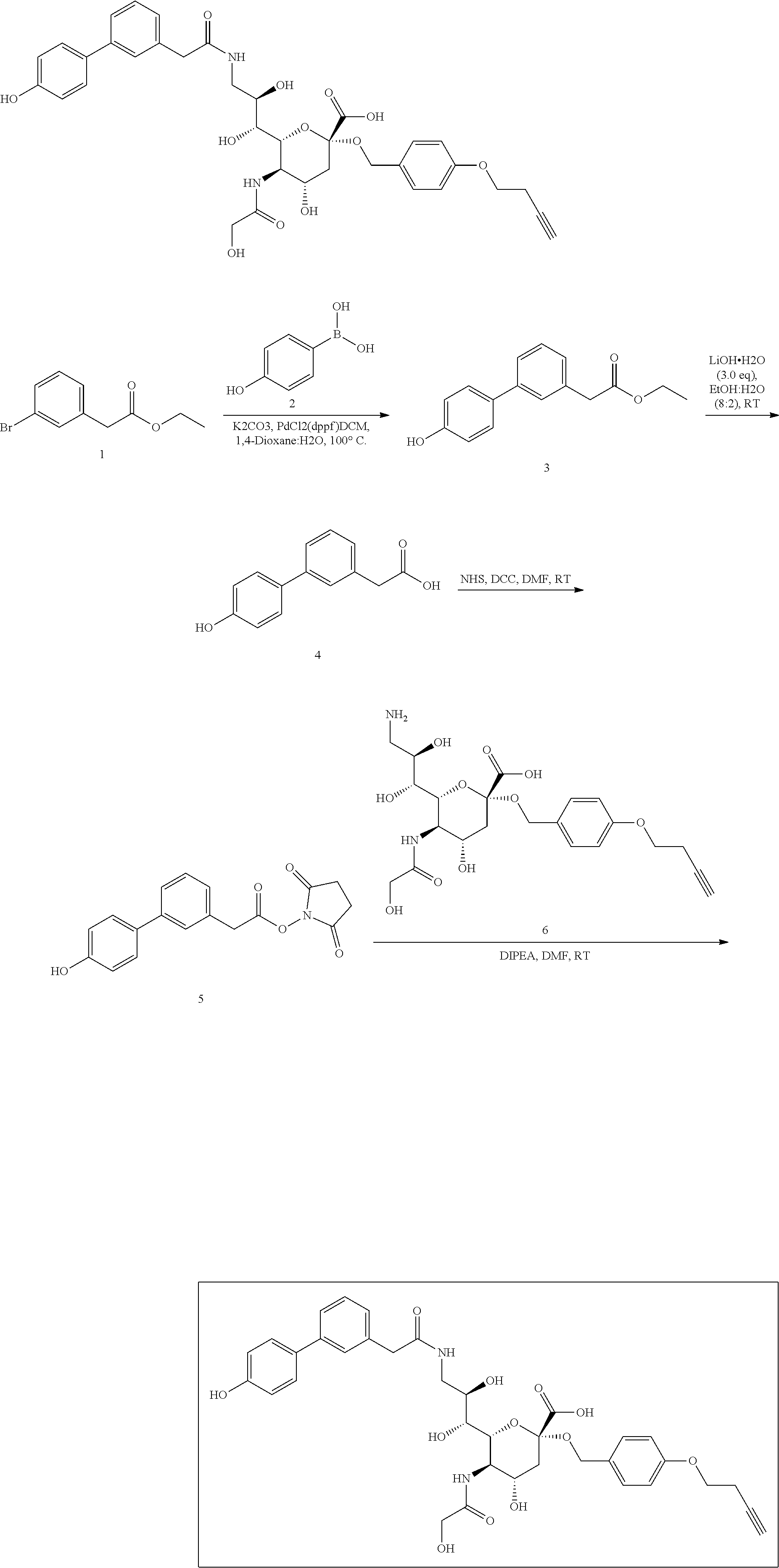

### Synthesis of ethyl 2-(3′-hydroxy-[1,1′-biphenyl]-3-yl)acetate (3)

To a stirred solution of ethyl 2-(3-bromophenyl)acetate (1, 1.00 g, 4.11 mmol) in degassed 1,4-dioxane (8.0 mL) and water (2.0 mL) were added potassium carbonate (1.71 g, 12.3 mmol) and [1,1′-Bis(diphenylphosphino)ferrocene]dichloropalladium(II) (0.168 g, 0.206 mmol) followed by (4-hydroxyphenyl)boronic acid (2, 0.851 g, 6.170 mmol) at room temperature and reaction mixture was stirred at 1000° C. for 12 h. Progress of the reaction was monitored by TLC and LCMS. After completion the reaction mixture was diluted with water and ethyl acetate. Organic layer was separated, dried over sodium sulphate, filtered and concentrated under reduced pressure to get crude. The crude was purified by column chromatography and desired compound was eluted with 15-20% ethyl acetate in heptane to afford ethyl 2-(4′-hydroxy-[1,1′-biphenyl]-3-yl)acetate (3) as off white solid. Yield: 0.70 g, 66%; LCMS (ESI) m/z 255.16 [M−1]⁻.

### Synthesis of 2-(4′-hydroxy-[1,1′-biphenyl]-3-yl)acetic acid (4)

To a stirred solution of ethyl 2-(4′-hydroxy-[1,1′-biphenyl]-3-yl)acetate (3, 0.70 g, 2.73 mmol) in ethanol (8.0 mL) was added solution of lithium hydroxide monohydrate (1.23 g, 29.40 mmol) in water (2.0 mL). The reaction mixture was stirred at room temperature for 5 h. Progress of the reaction was monitored by TLC and LCMS. After completion the reaction mixture was concentrated under reduced pressure and the mixture was treated with 1N HCl solution and the suspension was filtered through sintered funnel and washed with water and diethyl ether to afford 2-(4′-hydroxy-[1,1′-biphenyl]-3-yl)acetic acid (4) as off white solid. Yield: 0.50 g, 80%; LCMS (ESI) m/z 227.00 [M−1]⁻.

### Synthesis of 2,5-dioxopyrrolidin-1-yl 2-(4′-hydroxy-[1,1′-biphenyl]-3-yl)acetate (5)

To a stirred solution of 2-(4′-hydroxy-[1,1′-biphenyl]-3-yl)acetic acid (4, 0.640 g, 0.207 mmol) in N, N-dimethylformamide (6.0 mL). 1-hydroxypyrrolidine-2,5-dione (0.323 g, 2.80 mmol) and N,N′-dicyclohexylmethanediimine (0.279 g, 2.80 mmol) was added at 0° C. and reaction mixture was stirred at room temperature for 4 h. Progress of the reaction was monitored by TLC and LCMS. After completion the reaction mixture was concentrated under reduced pressure and residue was dissolved in ethyl acetate and filtered through sintered funnel and washed with ethyl acetate to afford 2,5-dioxopyrrolidin-1-yl 2-(4′-hydroxy-[1,1′-biphenyl]-3-yl)acetate (5) as off white solid. Yield: 0.600 g, 66%; LCMS (ESI) m/z 324.05 [M−1]⁻.

### Synthesis of (2R,4S,5R,6R)-2-((4-(but-3-yn-1-yloxy)benzyl)oxy)-6-((1R,2R)-1,2-dihydroxy-3-(2-(4′-hydroxy-[1,1′-biphenyl]-3-yl)acetamido)propyl)-4-hydroxy-5-(2-hydroxyacetamido)tetrahydro-2H-pyran-2-carboxylic acid (Compound 44)

To a stirred solution of (2R,4S,5R,6R)-6-((1R,2R)-3-amino-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (6, 0.20 g, 0.414 mmol) and 2,5-dioxopyrrolidin-1-yl 2-(4′-hydroxy-[1,1′-biphenyl]-3-yl)acetate (5, 0.107 g, 0.331 mmol) in dimethylformamide (2.0 mL) was added ethylbis(propan-2-yl)amine (0.36 mL, 2.07 mmol) at 0° C. and reaction mixture was stirred at room temperature for 4 h. Progress of the reaction was monitored by LCMS. After completion the reaction mixture was concentrated under reduced pressure to get crude. The crude material was purified by prep HPLC purification using 0.1% TFA in water/Acetonitrile to afford (2R,4S,5R,6R)-2-((4-(but-3-yn-1-yloxy)benzyl)oxy)-6-((1R,2R)-1,2-dihydroxy-3-(2-(4′-hydroxy-[1,1′-biphenyl]-3-yl)acetamido)propyl)-4-hydroxy-5-(2-hydroxyacetamido)tetrahydro-2H-pyran-2-carboxylic acid (Compound 44) as off white solid. Yield: 0.066 g, 23%; LCMS m/z 693.40 [M+H]+. 1H NMR (400 MHz, methanol-d4): δ 7.87 (d, J=7.6 Hz, 1H), 7.51-7.50 (m, 1H), 7.46-7.42 (m, 2H), 7.40-7.38 (m, 1H), 7.30 (t, J=7.6 Hz, 1H), 7.21-7.19 (m, 3H), 6.87-6.82 (m, 4H), 4.70 (d, J=11.2 Hz, 1H), 4.38 (d, J=11.2 Hz, 1H), 4.03 (t, J=6.4 Hz, 2H), 4.01 (bs, 2H), 3.94-3.89 (m, 1H), 3.86-3.71 (m, 3H), 3.60 (m, 1H), 3.59 (bs, 2H), 3.40 (dd, J=8.8 Hz and 1.6 Hz, 1H), 3.26-3.21 (m, 1H), 2.78 (dd, J=12.8 Hz and 4.0 Hz, 1H), 2.63 (dt, J=6.8 Hz & 2.8 Hz, 2H), 2.32 (t, J=2.8 Hz, 1H), 1.82-1.74 (m, 1H

### (2S,4S,5R,6R)-2-((4-(but-3-yn-1-yloxy)benzyl)thio)-6-((1R,2R)-1,2-dihydroxy-3-(2-(4-hydroxyphenyl)acetamido)propyl)-4-hydroxy-5-(2-hydroxyacetamido)tetrahydro-2H-pyran-2-carboxylic acid (Compound 148)

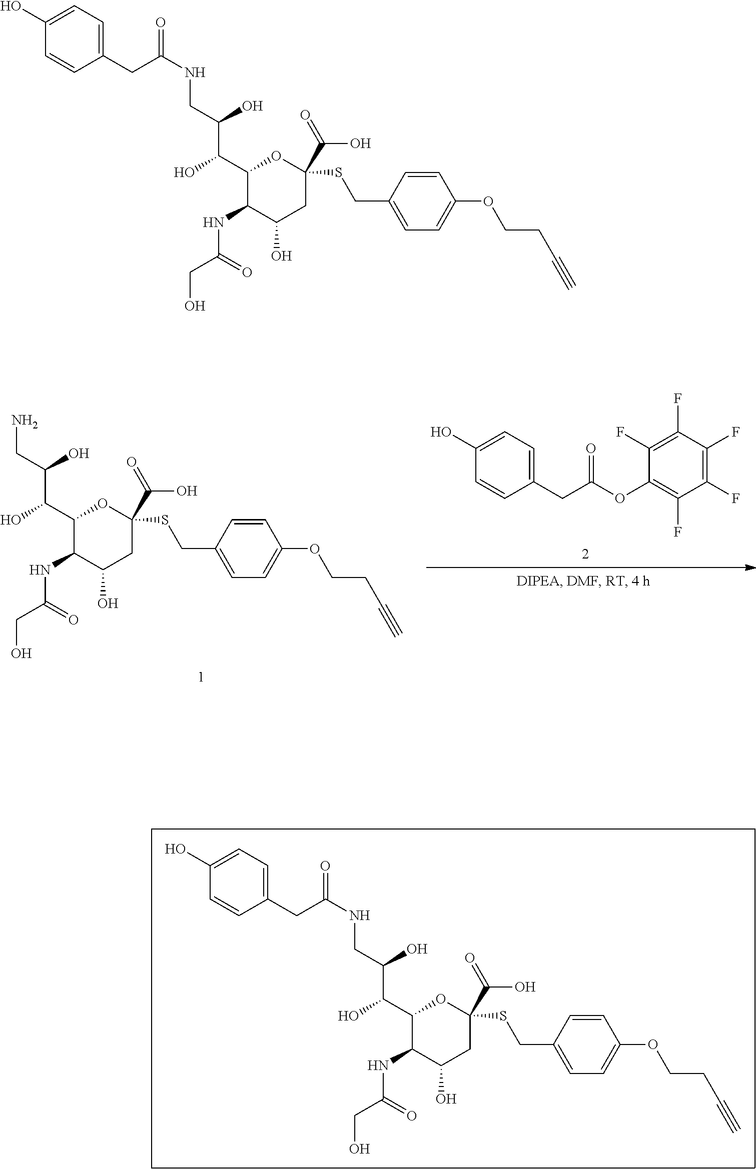 [SMILES: C#CCCOc1ccc(CS[C@]2(C(=O)O)C[C@H](O)[C@@H](NC(=O)CO)[C@H]([C@H](O)[C@H](O)CNC(=O)Cc3ccc(O)cc3)O2)cc1 ; C#CCCOc1ccc(CS[C@]2(C(=O)O)C[C@H](O)[C@@H](NC(=O)CO)[C@H]([C@H](O)[C@H](O)CN)O2)cc1 ; O=C(Cc1ccc(O)cc1)Oc1c(F)c(F)c(F)c(F)c1F ; C#CCCOc1ccc(CS[C@]2(C(=O)O)C[C@H](O)[C@@H](NC(=O)CO)[C@H]([C@H](O)[C@H](O)CNC(=O)Cc3ccc(O)cc3)O2)cc1]

### Synthesis of (2S,4S,5R,6R)-2-((4-(but-3-yn-1-yloxy)benzyl)thio)-6-((1R,2R)-1,2-dihydroxy-3-(2-(4-hydroxyphenyl)acetamido)propyl)-4-hydroxy-5-(2-hydroxyacetamido)tetrahydro-2H-pyran-2-carboxylic acid (Compound 148)

To a stirred solution of (2S,4S,5R,6R)-6-((1R,2R)-3-amino-1,2-dihydroxypropyl)-2-((4-(but-3-yn-1-yloxy)benzyl)thio)-4-hydroxy-5-(2-hydroxyacetamido)tetrahydro-2H-pyran-2-carboxylic acid (1, 0.20 g, 0.401 mmol) and perfluorophenyl 2-(4-hydroxyphenyl)acetate (2, 0.102 g, 0.320 mmol) in N,N-dimethylformamide (3.0 mL) was added ethylbis(propan-2-yl)amine (0.349 mL, 2.01 mmol) at 0° C. and reaction mixture was stirred at room temperature for 4 h. Progress of the reaction was monitored by LCMS. After completion the reaction mixture was concentrated under reduced pressure to get crude as yellow solid. The crude was purified by prep HPLC using 100% ACN in water with 0.1% TFA as buffer to afford (2S,4S,5R,6R)-2-((4-(but-3-yn-1-yloxy)benzyl)thio)-6-((1R,2R)-1,2-dihydroxy-3-(2-(4-hydroxyphenyl)acetamido)propyl)-4-hydroxy-5-(2-hydroxyacetamido)tetrahydro-2H-pyran-2-carboxylic acid (Compound 148) as a white solid. Yield: 0.053 g, 21%; LCMS m/z 633.45 [M+H]+; 1H NMR (400 MHz, methanol-d4): δ 7.94 (d, J=8.0 Hz, 1H), 7.24-7.20 (m, 2H), 7.10-7.07 (m, 2H), 6.87-6.83 (m, 2H), 6.71-6.67 (m, 2H), 4.06 (t, J=6.8 Hz, 2H), 4.02 (s, 2H), 3.98 (d, J=12.4 Hz, 1H), 3.91-3.78 (m, 4H), 3.65-3.59 (m, 2H), 3.43 (s, 2H), 3.36 (dd, J=8.8 Hz & 1.6 Hz, 1H), 3.25-3.20 (m, 1H), 2.77 (dd, J=12.4 Hz & 4 Hz, 1H), 2.63 (dt, J=6.8 Hz & 2.8 Hz, 2H), 2.32 (t, J=2.8 Hz, 1H), 1.82-1.76 (m, 1H).

### (2R,4S,5R,6R)-6-((1R,2R)-3-(2-(3-acetamidophenyl)acetamido)-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (Compound 164)

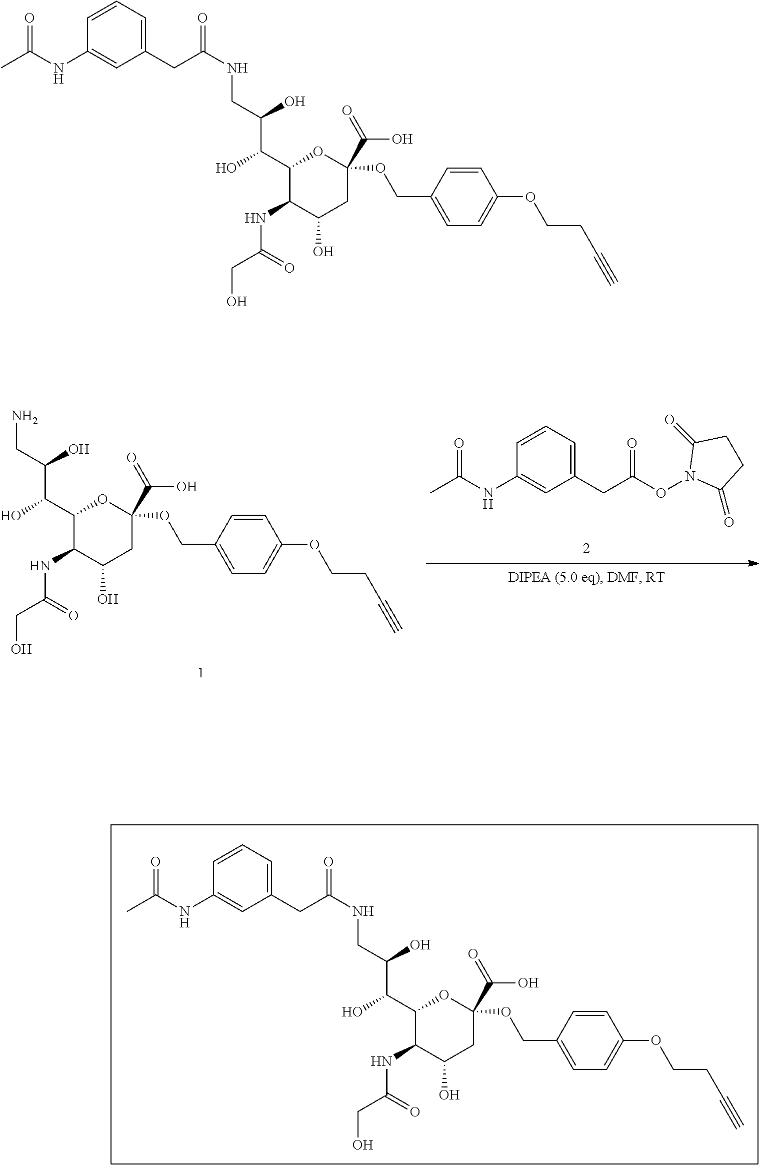

### Synthesis of (2R,4S,5R,6R)-6-((1R,2R)-3-(2-(3-acetamidophenyl)acetamido)-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (Compound 164)

To a stirred solution of (2R,4S,5R,6R)-6-((1R,2R)-3-amino-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (1, 0.20 g, 0.415 mmol) and 2,5-dioxopyrrolidin-1-yl 2-(3-acetamidophenyl)acetate (2, 0.120 g, 0.415 mmol) in N,N-dimethylformamide (4.0 mL) was added ethylbis(propan-2-yl)amine (0.36 mL, 2.07 mmol) at 0° C. and reaction mixture was stirred at room temperature for 4 h. Progress of reaction was monitored by TLC and LCMS and after completion, the reaction mixture was concentrated under reduced pressure to get crude which was purified by prep HPLC using ACN water with 0.1% TFA as additive that afforded (2R,4S,5R,6R)-6-((1R,2R)-3-(2-(3-acetamidophenyl)acetamido)-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (Compound 164) as a white solid. Yield: 0.043 g, 16%; LCMS m/z 658.35 [M+H]+; 1H NMR (400 MHz, methanol-d4): δ 7.45 (d, J=8.8 Hz, 2H), 7.25-7.20 (m, 3H), 7.04 (d, J=7.6 Hz, 1H), 6.89-6.86 (m, 2H), 4.74 (d, J=10.8 Hz, 1H), 4.42 (d, J=11.2 Hz, 1H), 4.06 (t, J=6.8 Hz, 2H), 4.02 (bs, 2H), 3.95-3.77 (m, 4H), 3.68-3.64 (m, 1H), 3.52 (bs, 2H), 3.41-3.39 (m, 1H), 3.33-3.31 (m, 1H), 2.78-2.74 (dd, J=4.0 Hz, 12.4 Hz, 1H), 2.65-2.61 (m, 2H), 2.32 (t, J=2.8 Hz, 1H), 2.08 (s, 3H), 1.79-1.73 (m, 1H).

### (2R,4S,5R,6R)-2-((4-(but-3-yn-1-yloxy)benzyl)oxy)-6-((1R,2R)-3-(2-(4-chloro-3-hydroxyphenyl)acetamido)-1,2-dihydroxypropyl)-4-hydroxy-5-(2-hydroxyacetamido)tetrahydro-2H-pyran-2-carboxylic acid (Compound 135)

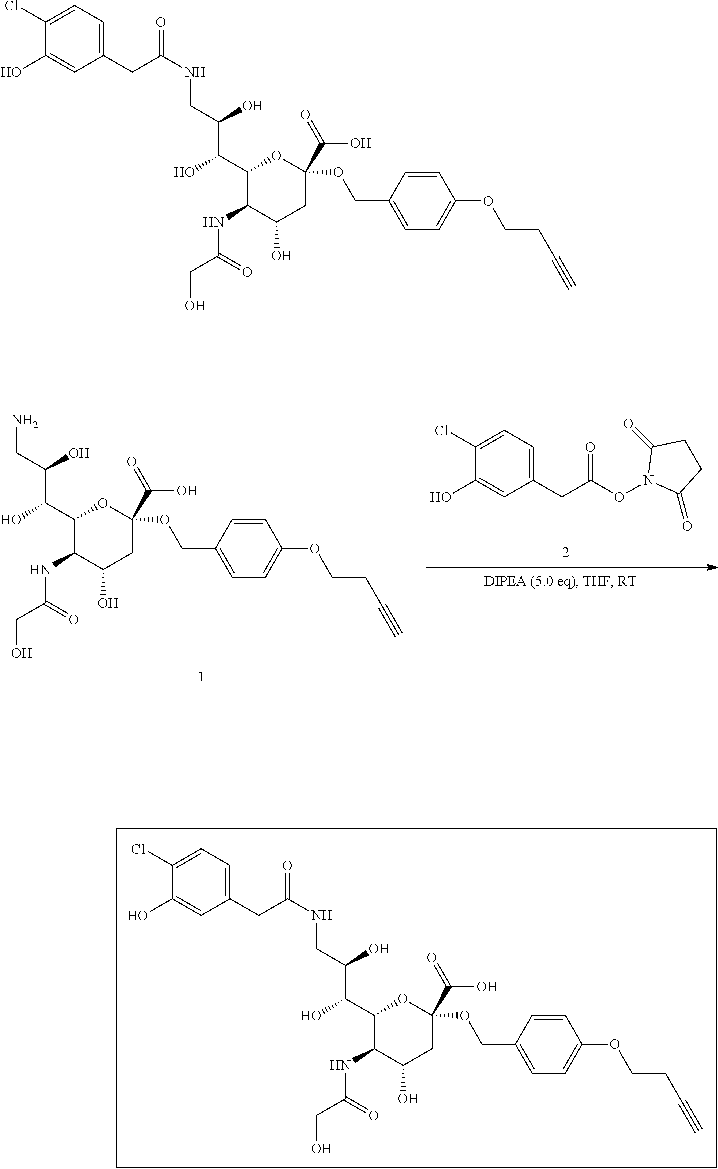 [SMILES: C#CCCOc1ccc(CO[C@]2(C(=O)O)C[C@H](O)[C@@H](NC(=O)CO)[C@H]([C@H](O)[C@H](O)CNC(=O)Cc3ccc(Cl)c(O)c3)O2)cc1 ; C#CCCOc1ccc(CO[C@]2(C(=O)O)C[C@H](O)[C@@H](NC(=O)CO)[C@H]([C@H](O)[C@H](O)CN)O2)cc1 ; O=C(Cc1ccc(Cl)c(O)c1)ON1C(=O)CCC1=O ; C#CCCOc1ccc(CO[C@]2(C(=O)O)C[C@H](O)[C@@H](NC(=O)CO)[C@H]([C@H](O)[C@H](O)CNC(=O)Cc3ccc(Cl)c(O)c3)O2)cc1]

### Synthesis of (2R,4S,5R,6R)-2-((4-(but-3-yn-1-yloxy)benzyl)oxy)-6-((1R,2R)-3-(2-(4-chloro-3-hydroxyphenyl)acetamido)-1,2-dihydroxypropyl)-4-hydroxy-5-(2-hydroxyacetamido)tetrahydro-2H-pyran-2-carboxylic acid (Compound 135)

To a stirred solution of (2R,4S,5R,6R)-6-((1R,2R)-3-amino-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (1, 0.20 g, 0.415 mmol) and 2,5-dioxopyrrolidin-1-yl 2-(4-chloro-3-hydroxyphenyl)acetate (2, 0.071 g, 0.415 mmol) in N,N-dimethylformamide (4.0 mL) was added ethylbis(propan-2-yl)amine (0.36 mL, 2.07 mmol) at 0° C. and reaction mixture was stirred at room temperature for 4 h. Progress of the reaction was monitored by TLC and LCMS and after completion the reaction mixture was concentrated under reduced pressure to get crude which was purified by prep HPLC purification using ACN-water with 0.1% TFA as additive that afforded (2R,4S,5R,6R)-2-((4-(but-3-yn-1-yloxy)benzyl)oxy)-6-((1R,2R)-3-(2-(4-chloro-3-hydroxyphenyl)acetamido)-1,2-dihydroxypropyl)-4-hydroxy-5-(2-hydroxyacetamido)tetrahydro-2H-pyran-2-carboxylic acid (Compound 135) as a white solid. Yield: 0.038 g, 14%; LCMS m/z 651.53 [M+H]+; 1H NMR (400 MHz, methanol-d4): δ 7.99-7.92 (m, 1H), 7.25 (d, J=8.4 Hz, 2H), 7.19 (d, J=8.4 Hz, 1H), 6.91-6.86 (m, 3H), 6.74 (d, J=2.0 Hz and 8.0 Hz, 1H), 4.75 (d, J=10.8 Hz, 1H), 4.41 (d, J=10.8 Hz, 1H), 4.07 (t, J=6.8 Hz, 2H), 4.02 (s, 2H), 3.99-3.79 (m, 4H), 3.68-3.67 (m, 1H), 3.42-3.39 (m, 3H), 3.29-3.23 (m, 1H), 2.78-2.74 (dd, J=4.0 Hz, 12.8 Hz, 1H), 2.65-2.61 (m, 2H), 2.32 (t, J=2.8 Hz, 1H), 1.77 (t, J=12.4 Hz, 1H).

### (2S,4S,5R,6R)-2-((4-(but-3-yn-1-yloxy)benzyl)thio)-6-((1R,2R)-3-(2-(3-chlorophenyl)acetamido)-1,2-dihydroxypropyl)-4-hydroxy-5-(2-hydroxyacetamido)tetrahydro-2H-pyran-2-carboxylic acid (Compound 120)

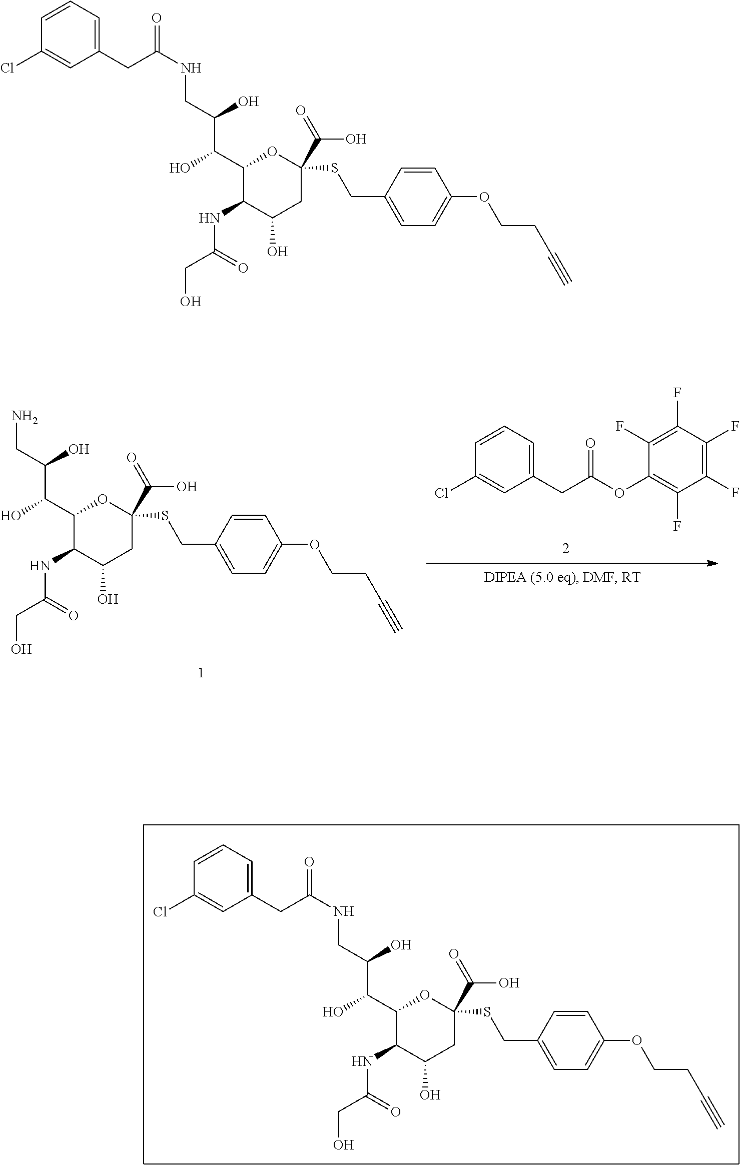 [SMILES: C#CCCOc1ccc(CS[C@]2(C(=O)O)C[C@H](O)[C@@H](NC(=O)CO)[C@H]([C@H](O)[C@H](O)CNC(=O)Cc3cccc(Cl)c3)O2)cc1 ; C#CCCOc1ccc(CS[C@]2(C(=O)O)C[C@H](O)[C@@H](NC(=O)CO)[C@H]([C@H](O)[C@H](O)CN)O2)cc1 ; O=C(Cc1cccc(Cl)c1)Oc1c(F)c(F)c(F)c(F)c1F ; C#CCCOc1ccc(CS[C@]2(C(=O)O)C[C@H](O)[C@@H](NC(=O)CO)[C@H]([C@H](O)[C@H](O)CNC(=O)Cc3cccc(Cl)c3)O2)cc1]

### Synthesis of (2S,4S,5R,6R)-2-((4-(but-3-yn-1-yloxy)benzyl)thio)-6-((1R,2R)-3-(2-(3-chlorophenyl)acetamido)-1,2-dihydroxypropyl)-4-hydroxy-5-(2-hydroxyacetamido)tetrahydro-2H-pyran-2-carboxylic acid (Compound 120)

To a stirred solution of (2R,4S,5R,6R)-6-[(1R,2R)-3-amino-1,2-dihydroxypropyl]-2-{[4-(but-3-yn-1-yloxy)phenyl]methoxy}-4-hydroxy-5-(2-hydroxyacetamido)oxane-2-carboxylic acid (1, 0.20 g, 0.401 mmol) and perfluorophenyl 2-(3-chlorophenyl)acetate (2, 0.107 g, 0.401 mmol) in N,N-dimethylformamide (4.0 mL) was added ethylbis(propan-2-yl)amine (0.35 mL, 2.01 mmol) at 0° C. and reaction mixture was stirred at room temperature for 4 h. Progress of the reaction was monitored by TLC and LCMS and after completion the reaction mixture was concentrated under reduced pressure to get crude which was purified by prep HPLC using ACN-water with 0.1% TFA as additive to afford (2S,4S,5R,6R)-2-((4-(but-3-yn-1-yloxy)benzyl)thio)-6-((1R,2R)-3-(2-(3-chlorophenyl)acetamido)-1,2-dihydroxypropyl)-4-hydroxy-5-(2-hydroxyacetamido)tetrahydro-2H-pyran-2-carboxylic acid (Compound 120) as a white solid. Yield: 0.018 g, 7%; LCMS m/z [M+H]+; 1H NMR (400 MHz, methanol-d4): δ 7.33 (bs, 1H), 7.22-7.16 (m, 5H), 6.84 (d, J=8.4 Hz, 2H), 4.06 (t, J=6.8 Hz, 2H), 4.01 (s, 2H), 3.99-3.96 (m, 1H), 3.88-3.77 (m, 4H), 3.68-3.64 (m, 2H), 3.52 (s, 2H), 3.38-3.35 (m, 1H), 3.23-3.18 (m, 1H), 2.80-2.76 (dd, J=4.0 Hz, 12.4 Hz, 1H), 2.65-2.61 (m, 1H), 2.31 (t, J=2.8 Hz, 1H), 1.80-1.75 (m, 1H).

### (2R,4S,5R,6R)-6-((1R,2R)-3-(2-(2-acetamidophenyl)acetamido)-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (Compound 163)

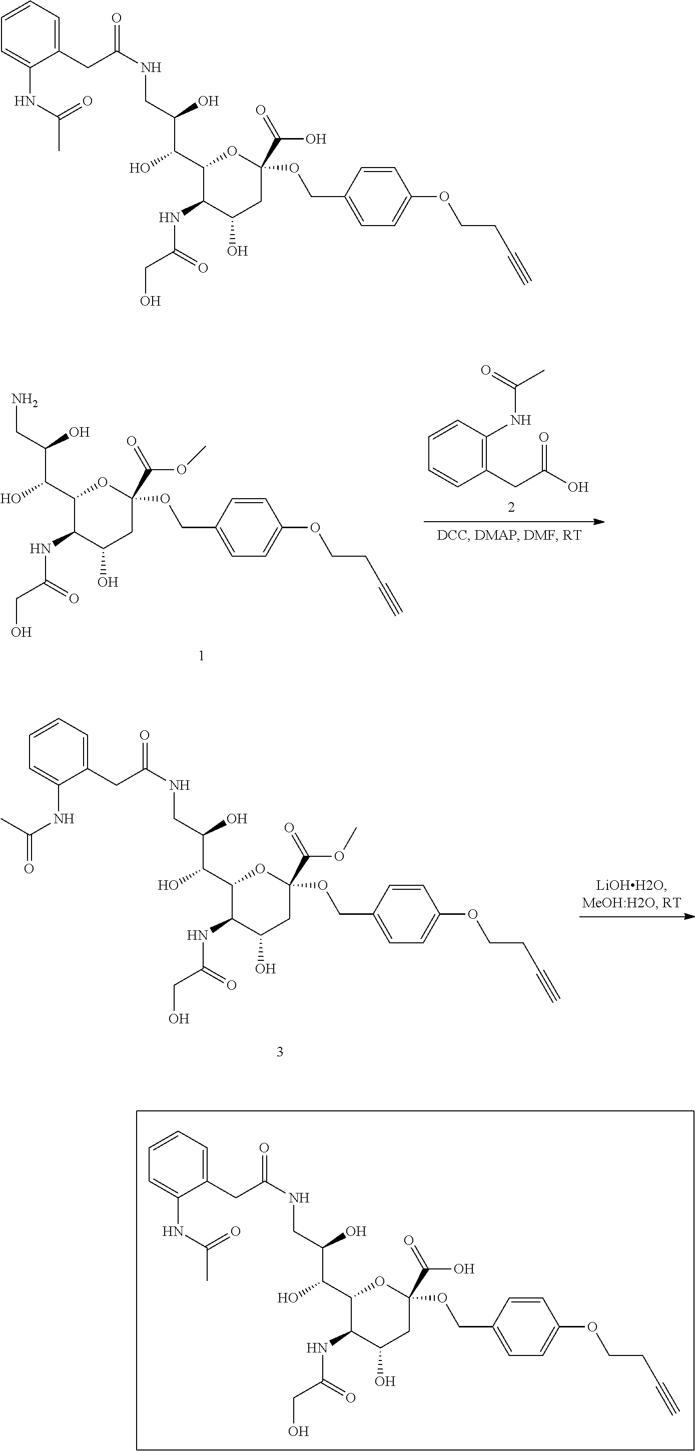

### Synthesis of methyl (2R,4S,5R,6R)-6-((1R,2R)-3-(2-(2-acetamidophenyl)acetamido)-1,2-dihydroxypropyl)-2-((4-(but-3-yn-1-yloxy)benzyl)oxy)-4-hydroxy-5-(2-hydroxyacetamido)tetrahydro-2H-pyran-2-carboxylate (3)

To a stirred solution of 2-(2-acetamidophenyl)acetic acid (2, 0.150 g, 0.776 mmol) in N,N-dimethylformamide (5.0 mL), N,N′-dicyclohexylcarbodiimide 0.320 g, 1.55 mmol), N,N-dimethylpyridin-4-amine (0.142 g, 1.16 mmol) and methyl (2R,4S,5R,6R)-6-((1R,2R)-3-amino-1,2-dihydroxypropyl)-2-((4-(but-3-yn-1-yloxy)benzyl)oxy)-4-hydroxy-5-(2-hydroxyacetamido)tetrahydro-2H-pyran-2-carboxylate (0.385 g, 0.776 mmol) was added at 0° C. and reaction mixture was stirred at room temperature for 12 h. Progress of the reaction was monitored by TLC and LCMS and after completion the reaction mixture was concentrated under reduced pressure to get crude. The crude was purified by column chromatography and desired compound eluted with 90-95% ethyl acetate in heptane to afford methyl (2R,4S,5R,6R)-6-((1R,2R)-3-(2-(2-acetamidophenyl)acetamido)-1,2-dihydroxypropyl)-2-((4-(but-3-yn-1-yloxy)benzyl)oxy)-4-hydroxy-5-(2-hydroxyacetamido)tetrahydro-2H-pyran-2-carboxylate (3) as a yellow solid. Yield: 0.300 g, 58%; LCMS (ESI) m/z 672.20 [M+H]+.

### Synthesis of (2R,4S,5R,6R)-6-((1R,2R)-3-(2-(2-acetamidophenyl)acetamido)-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (Compound 163)

To a stirred solution of methyl (2R,4S,5R,6R)-6-((1R,2R)-3-(2-(2-acetamidophenyl)acetamido)-1,2-dihydroxypropyl)-2-((4-(but-3-yn-1-yloxy)benzyl)oxy)-4-hydroxy-5-(2-hydroxyacetamido)tetrahydro-2H-pyran-2-carboxylate (3, 0.30 g, 0.446 mmol) in methanol (3.0 ml) was added solution of Lithium hydroxide monohydrate (0.038 g, 0.893 mmol) in water (0.50 ml). The reaction mixture was stirred at room temperature for 6 h. The reaction mixture was monitored by LCMS. After completion the reaction mixture was treated with Dowex 50, H⁺) to pH˜6 and the suspension was filtered and washed with methanol. The filtrate was concentrated under reduced pressure to get crude. The crude material was purified by prep HPLC purification using in water/Acetonitrile solvent system with 0.1% TFA as additive to afford (2R,4S,5R,6R)-6-((1R,2R)-3-(2-(2-acetamidophenyl)acetamido)-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (Compound 163) as a white solid. Yield: 0.052 g; 18%; LCMS (ESI) m/z 658.40 [M+H]+; ¹H NMR (400 MHz, MeOD-d4): δ 7.94 (d, J=8.0 Hz, 1H), 7.53 (d, J=7.6 Hz, 1H), 7.31 (d, J=7.6 Hz, 1H), 7.27-7.24 (m, 3H), 7.16 (t, J=7.2 Hz, 1H), 6.90 (d, J=8.8 Hz, 2H), 4.75 (d, J=11.2 Hz, 1H), 4.42 (d, J=10.8 Hz, 1H), 4.09 (t, J=6.8 Hz, 2H), 4.02 (s, 2H), 3.94 (td, J=8.4 Hz & 3.2 Hz, 1H), 3.89-3.85 (m, 2H), 3.79 (m, 1H), 3.69 (dd, J=13.6 Hz & 2.8 Hz, 1H), 3.59 (s, 2H), 3.41 (d, J=8.8 Hz, 1H), 3.29-3.24 (m, 1H), 2.77 (dd, J=12.4 Hz & 3.6 Hz, 1H), 2.66 (td, J=6.8 Hz & 2.8 Hz, 2H), 2.34 (t, J=2.4 Hz, 1H), 2.17 (s, 3H), 1.79 (t, J=12.4 Hz, 1H).

### (2R,4S,5R,6R)-6-((1R,2R)-3-(2-(1H-indazol-6-yl)acetamido)-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (Compound 10)

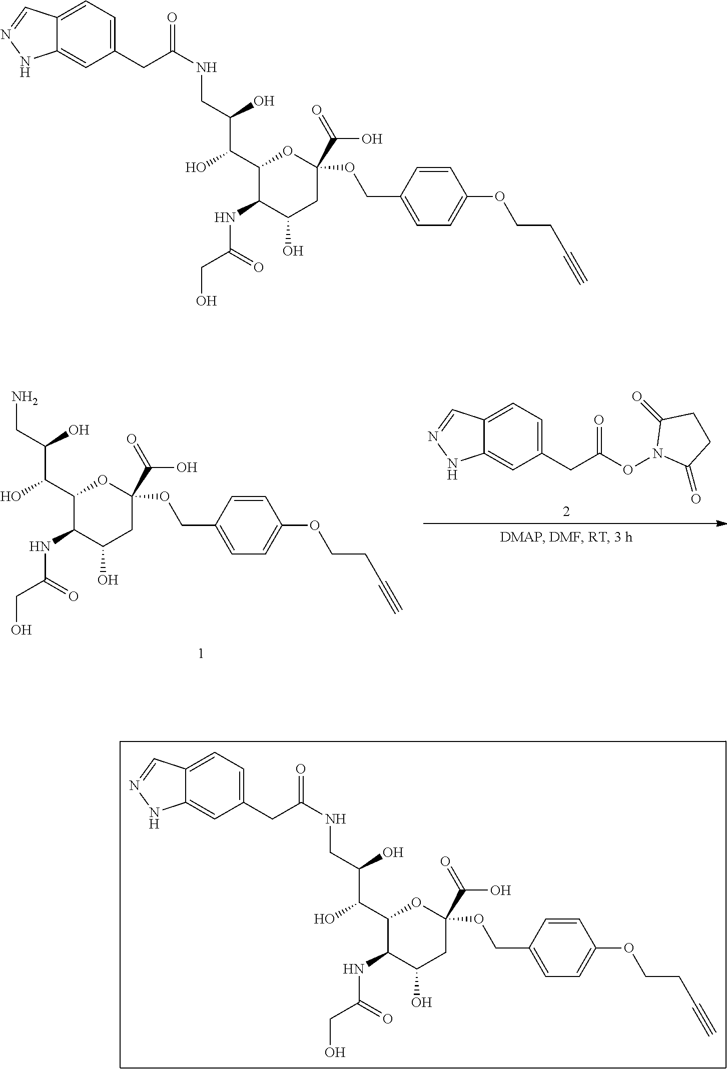 [SMILES: C#CCCOc1ccc(CO[C@]2(C(=O)O)C[C@H](O)[C@@H](NC(=O)CO)[C@H]([C@H](O)[C@H](O)CNC(=O)Cc3ccc4cn[nH]c4c3)O2)cc1 ; C#CCCOc1ccc(CO[C@]2(C(=O)O)C[C@H](O)[C@@H](NC(=O)CO)[C@H]([C@H](O)[C@H](O)CN)O2)cc1 ; O=C(Cc1ccc2cn[nH]c2c1)ON1C(=O)CCC1=O ; C#CCCOc1ccc(CO[C@]2(C(=O)O)C[C@H](O)[C@@H](NC(=O)CO)[C@H]([C@H](O)[C@H](O)CNC(=O)Cc3ccc4cn[nH]c4c3)O2)cc1]

### Synthesis of (2R,4S,5R,6R)-6-((1R,2R)-3-(2-(1H-indazol-6-yl)acetamido)-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (Compound 10)

To a stirred solution of (2R,4S,5R,6R)-6-((1R,2R)-3-amino-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (1, 0.20 g, 0.415 mmol) and 2,5-dioxopyrrolidin-1-yl 2-(1H-indazol-6-yl)acetate (2, 0.110 g, 0.415 mmol) in N,N-dimethylformamide (3.0 mL) was added ethylbis(propan-2-yl)amine (0.361 mL, 2.07 mmol) at 0° C. and reaction mixture was stirred at room temperature for 4 h. Progress of the reaction was monitored by LCMS. After completion the reaction mixture was concentrated under reduced pressure to get crude as yellow solid. The crude was purified by prep HPLC using 100% ACN in water with 0.1% TFA as buffer to afford (2R,4S,5R,6R)-6-((1R,2R)-3-(2-(1H-indazol-6-yl)acetamido)-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (Compound 10) as a white solid. Yield: 0.054 g, 20%; LCMS m/z 641.50 [M+H]+; 1H NMR (400 MHz, methanol-d4): δ 7.97 (s, 1H), 7.68 (d, J=8.4 Hz, 1H), 7.50 (s, 1H), 7.25 (d, J=8.8 Hz, 2H), 7.12 (dd, J=8.4 Hz & 0.8 Hz, 1H), 6.87 (d, J=8.4 Hz, 2H), 4.73 (d, J=10.8 Hz, 1H), 4.43 (d, J=10.8 Hz, 1H), 4.08 (t, J=6.8 Hz, 2H), 4.03 (s, 2H), 3.96 (dt, J=8.4 Hz & 3.2 Hz, 1H), 3.90-3.76 (m, 3H), 3.72-3.68 (m, 3H), 3.41 (dd, J=8.8 Hz & 1.2 Hz, 1H), 3.30-3.24 (m, 1H), 2.82 (dd, J=12.4 Hz & 4.4 Hz, 1H), 2.65 (dt, J=6.8 Hz & 2.4 Hz, 2H), 2.34 (t, J=2.8 Hz, 1H), 1.73 (t, J=12 Hz, 1H).

### (2R,4S,5R,6R)-6-((1R,2R)-3-(2-(1H-benzo[d]imidazol-6-yl)acetamido)-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (Compound 8)

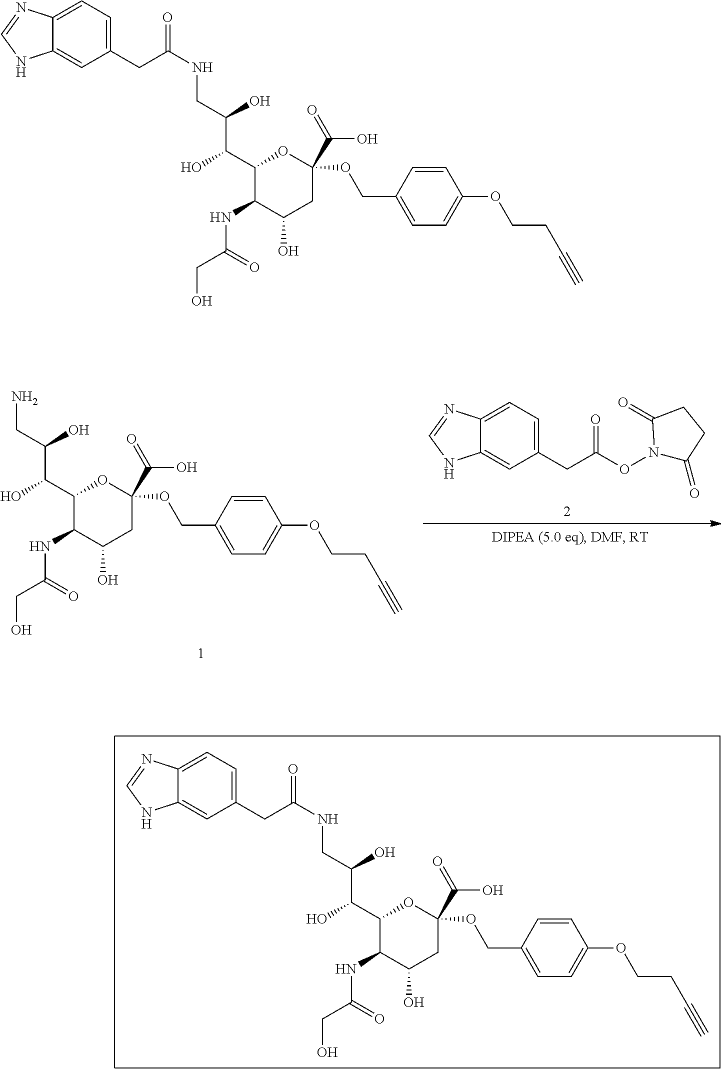 [SMILES: C#CCCOc1ccc(CO[C@]2(C(=O)O)C[C@H](O)[C@@H](NC(=O)CO)[C@H]([C@H](O)[C@H](O)CNC(=O)Cc3ccc4nc[nH]c4c3)O2)cc1 ; C#CCCOc1ccc(CO[C@]2(C(=O)O)C[C@H](O)[C@@H](NC(=O)CO)[C@H]([C@H](O)[C@H](O)CN)O2)cc1 ; O=C(Cc1ccc2nc[nH]c2c1)ON1C(=O)CCC1=O ; C#CCCOc1ccc(CO[C@]2(C(=O)O)C[C@H](O)[C@@H](NC(=O)CO)[C@H]([C@H](O)[C@H](O)CNC(=O)Cc3ccc4nc[nH]c4c3)O2)cc1]

### Synthesis of (2R,4S,5R,6R)-6-((1R,2R)-3-(2-(1H-benzo[d]imidazol-6-yl)acetamido)-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (Compound 8)

To a stirred solution of (2R,4S,5R,6R)-6-((1R,2R)-3-amino-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (1, 0.10 g, 0.207 mmol) and 2,5-dioxopyrrolidin-1-yl 2-(1H-benzo[d]imidazol-6-yl)acetate (2, 0.045 g, 0.166 mmol) in N,N-dimethylformamide (2.0 mL) was added ethylbis(propan-2-yl)amine (0.18 mL, 1.04 mmol) at 0° C. and reaction mixture was stirred at room temperature for 4 h. Progress of the reaction was monitored by TLC and LCMS and after completion the reaction mixture was concentrated under reduced pressure to get crude which was purified by prep HPLC using ACN-water with 0.1% TFA as additive to afford (2R,4S,5R,6R)-6-((1R,2R)-3-(2-(1H-benzo[d]imidazol-6-yl)acetamido)-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (Compound 8) as a white solid. Yield: 0.020 g, 15.06%; LCMS m/z 641.35 [M+H]+; 1H NMR (400 MHz, methanol-d4): δ 9.05 (s, 1H), 7.76 (bs, 1H), 7.70 (d, J=8.4 Hz, 1H), 7.52 (d, J=8.0 Hz, 1H), 7.19 (d, J=8.4 Hz, 2H), 6.84 (d, J=8.4 Hz, 1H), 4.67 (d, J=10.8 Hz, 1H), 4.38 (d, J=10.8 Hz, 1H), 4.05 (t, J=6.8 Hz, 2H), 4.01 (bs, 2H), 3.97-3.67 (m, 7H), 3.41-3.94 (m, 1H), 3.31-3.23 (m, 1H), 2.82-2.78 (dd, J=4.0 Hz, 12.4 Hz, 1H), 2.65-2.61 (m, 1H), 2.33 (t, J=2.8 Hz, 1H), 1.71 (t, J=12.4 Hz, 1H).

### (2R,4S,5R,6R)-2-((4-(but-3-yn-1-yloxy)benzyl)oxy)-6-((1R,2R)-1,2-dihydroxy-3-(2-(2-oxo-2,3-dihydro-1H-benzo[d]imidazol-5-yl)acetamido)propyl)-4-hydroxy-5-(2-hydroxyacetamido)tetrahydro-2H-pyran-2-carboxylic acid (Compound 7)

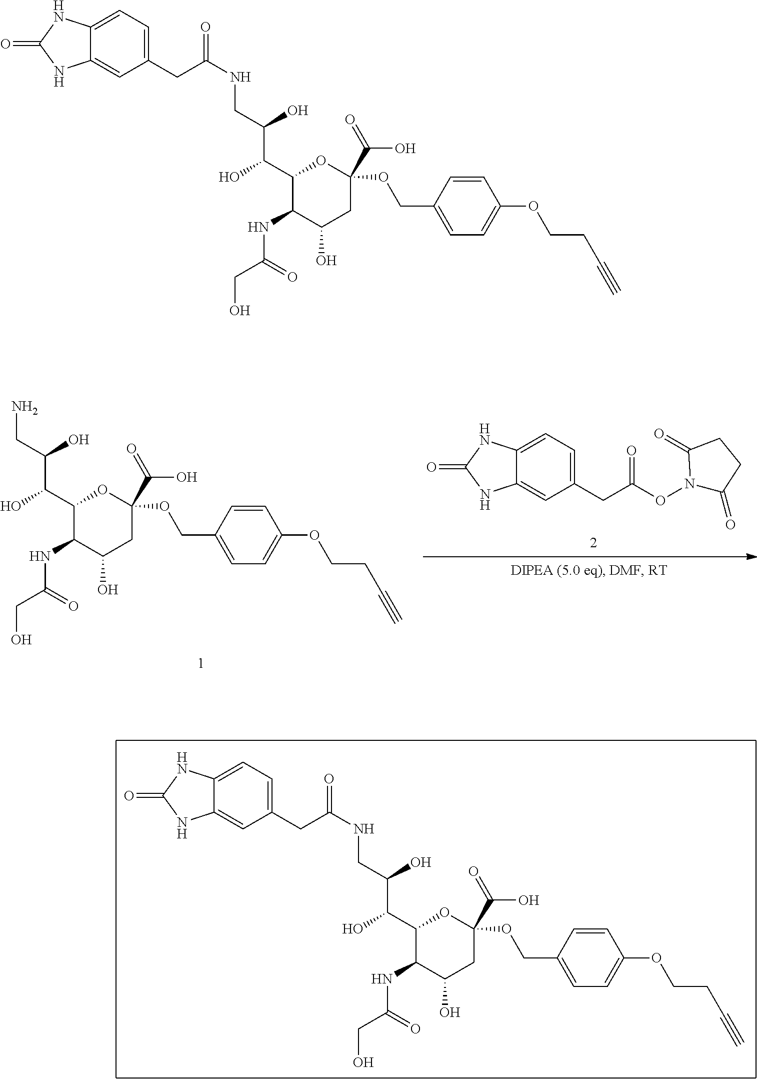 [SMILES: C#CCCOc1ccc(CO[C@]2(C(=O)O)C[C@H](O)[C@@H](NC(=O)CO)[C@H]([C@H](O)[C@H](O)CNC(=O)Cc3ccc4[nH]c(=O)[nH]c4c3)O2)cc1 ; C#CCCOc1ccc(CO[C@]2(C(=O)O)C[C@H](O)[C@@H](NC(=O)CO)[C@H]([C@H](O)[C@H](O)CN)O2)cc1 ; O=C(Cc1ccc2[nH]c(=O)[nH]c2c1)ON1C(=O)CCC1=O ; C#CCCOc1ccc(CO[C@]2(C(=O)O)C[C@H](O)[C@@H](NC(=O)CO)[C@H]([C@H](O)[C@H](O)CNC(=O)Cc3ccc4[nH]c(=O)[nH]c4c3)O2)cc1]

### Synthesis of (2R,4S,5R,6R)-2-((4-(but-3-yn-1-yloxy)benzyl)oxy)-6-((1R,2R)-1,2-dihydroxy-3-(2-(2-oxo-2,3-dihydro-1H-benzo[d]imidazol-5-yl)acetamido)propyl)-4-hydroxy-5-(2-hydroxyacetamido)tetrahydro-2H-pyran-2-carboxylic acid (Compound 7)

To a stirred solution of (2R,4S,5R,6R)-6-((1R,2R)-3-amino-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (1, 0.110 g, 0.228 mmol) and 2,5-dioxopyrrolidin-1-yl 2-(2-oxo-2,3-dihydro-1H-benzo[d]imidazol-5-yl)acetate (2, 0.053 g, 0.182 mmol) in N,N-dimethylformamide (3.0 mL) was added ethylbis(propan-2-yl)amine (0.198 mL, 1.14 mmol) at 0° C. and reaction mixture was stirred at room temperature for 4 h. Progress of reaction was monitored by TLC and LCMS and after completion the reaction mixture was concentrated under reduced pressure to get crude which was purified by prep HPLC using ACN-water with 0.1% TFA as additive to afford (2R,4S,5R,6R)-2-((4-(but-3-yn-1-yloxy)benzyl)oxy)-6-((1R,2R)-1,2-dihydroxy-3-(2-(2-oxo-2,3-dihydro-1H-benzo[d]imidazol-5-yl)acetamido)propyl)-4-hydroxy-5-(2-hydroxyacetamido)tetrahydro-2H-pyran-2-carboxylic acid (Compound 7) as a white solid. Yield: 0.007 g, 5%; LCMS m/z 657.35 [M+H]+; 1H NMR (400 MHz, methanol-d4): δ 7.91-7.88 (m, 1H), 7.29-7.23 (m, 2H), 7.02 (s, 1H), 6.98-6.93 (m, 2H), 6.87-6.83 (m, 2H), 4.72 (d, J=10.8 Hz, 1H), 4.43 (d, J=10.8 Hz, 1H), 4.06 (d, J=6.8 Hz, 2H), 4.02 (s, 2H), 3.96-3.91 (m, 1H), 3.82-3.64 (m, 4H), 3.53 (s, 2H), 3.37-3.35 (m, 1H), 3.23-3.18 (m, 1H), 2.88-2.84 (dd, J=4.0 Hz, 12.0 Hz, 1H), 2.69-2.53 (m, 2H), 2.31 (t, J=2.4 Hz, 1H), 1.64 (t, J=12.0 Hz, 1H).

### (2R,4S,5R,6R)-6-((1R,2R)-3-(2-(1H-benzo[d][1,2,3]triazol-6-yl)acetamido)-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (Compound 9)

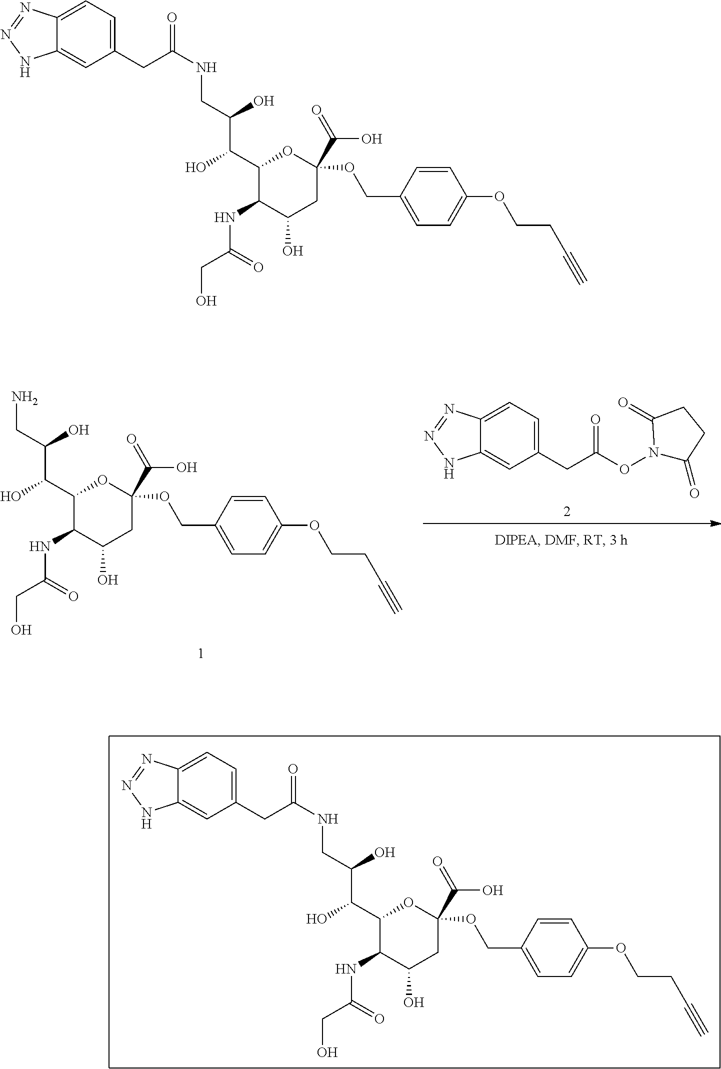 [SMILES: C#CCCOc1ccc(CO[C@]2(C(=O)O)C[C@H](O)[C@@H](NC(=O)CO)[C@H]([C@H](O)[C@H](O)CNC(=O)Cc3ccc4nn[nH]c4c3)O2)cc1 ; C#CCCOc1ccc(CO[C@]2(C(=O)O)C[C@H](O)[C@@H](NC(=O)CO)[C@H]([C@H](O)[C@H](O)CN)O2)cc1 ; O=C(Cc1ccc2nn[nH]c2c1)ON1C(=O)CCC1=O ; C#CCCOc1ccc(CO[C@]2(C(=O)O)C[C@H](O)[C@@H](NC(=O)CO)[C@H]([C@H](O)[C@H](O)CNC(=O)Cc3ccc4nn[nH]c4c3)O2)cc1]

### Synthesis of (2R,4S,5R,6R)-6-((1R,2R)-3-(2-(1H-benzo[d][1,2,3]triazol-6-yl)acetamido)-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (Compound 9)

To a stirred solution of (2R,4S,5R,6R)-6-((1R,2R)-3-amino-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (1, 0.20 g, 0.415 mmol) and 2,5-dioxopyrrolidin-1-yl 2-(1H-benzo[d][1,2,3]triazol-6-yl)acetate (2, 0.114 g, 0.415 mmol) in N,N-dimethylformamide (3.0 mL) was added ethylbis(propan-2-yl)amine (0.361 mL, 2.07 mmol) at 0° C. and reaction mixture was stirred at room temperature for 4 h. Progress of the reaction was monitored by LCMS and after completion the reaction mixture was concentrated under reduced pressure to get crude as yellow solid. The crude was purified by prep HPLC using 100% ACN in water with 0.1% TFA as buffer to obtain (2R,4S,5R,6R)-6-((1R,2R)-3-(2-(1H-benzo[d][1,2,3]triazol-6-yl)acetamido)-1,2-dihydroxypropyl)-2-((4-(but-3-yn-1-yloxy)benzyl)oxy)-4-hydroxy-5-(2-ydroxyacetamido)tetrahydro-2H-pyran-2-carboxylic acid (Compound 9) as a white solid. Yield: 0.048 g, 18%; LCMS m/z 642.40 [M+H]+; 1H NMR (400 MHz, methanol-d4): δ 7.93 (d, J=7.6 Hz, 1H), 7.81-7.79 (m, 2H), 7.45-7.42 (m, 2H), 7.22 (d, J=8.8 Hz, 2H), 6.86 (d, J=8.4 Hz, 2H), 4.71 (d, J=10.8 Hz, 21), 4.39 (d, J=10.8 Hz, 1H), 4.06 (t, J=6.8 Hz, 2H), 4.01 (s, 2H), 3.96-3.76 (m, 4H), 3.73 (s, 2H), 3.70 (dd, J=14 Hz & 2.8 Hz, 1H), 3.42 (dd, J=8.8 Hz & 1.2 Hz, 1H), 3.31-3.30 (m, 1H), 2.76 (dd, J=12.4 Hz & 4.0 Hz, 1H), 2.64 (dt, J=6.8 Hz & 2.8 Hz, 2H), 2.33 (t, J=2.8 Hz, 1H), 1.78 (t, J=12 Hz, 1H).

### (2R,4S,5R,6R)-6-((1R,2R)-3-(2-(4-chlorophenyl)acetamido)-1,2-dihydroxypropyl)-4-hydroxy-5-(2-hydroxyacetamido)-2-((4-((((prop-2-yn-1-yloxy)carbonyl)amino)methyl)benzyl)oxy)tetrahydro-2H-pyran-2-carboxylic acid (Compound 117)

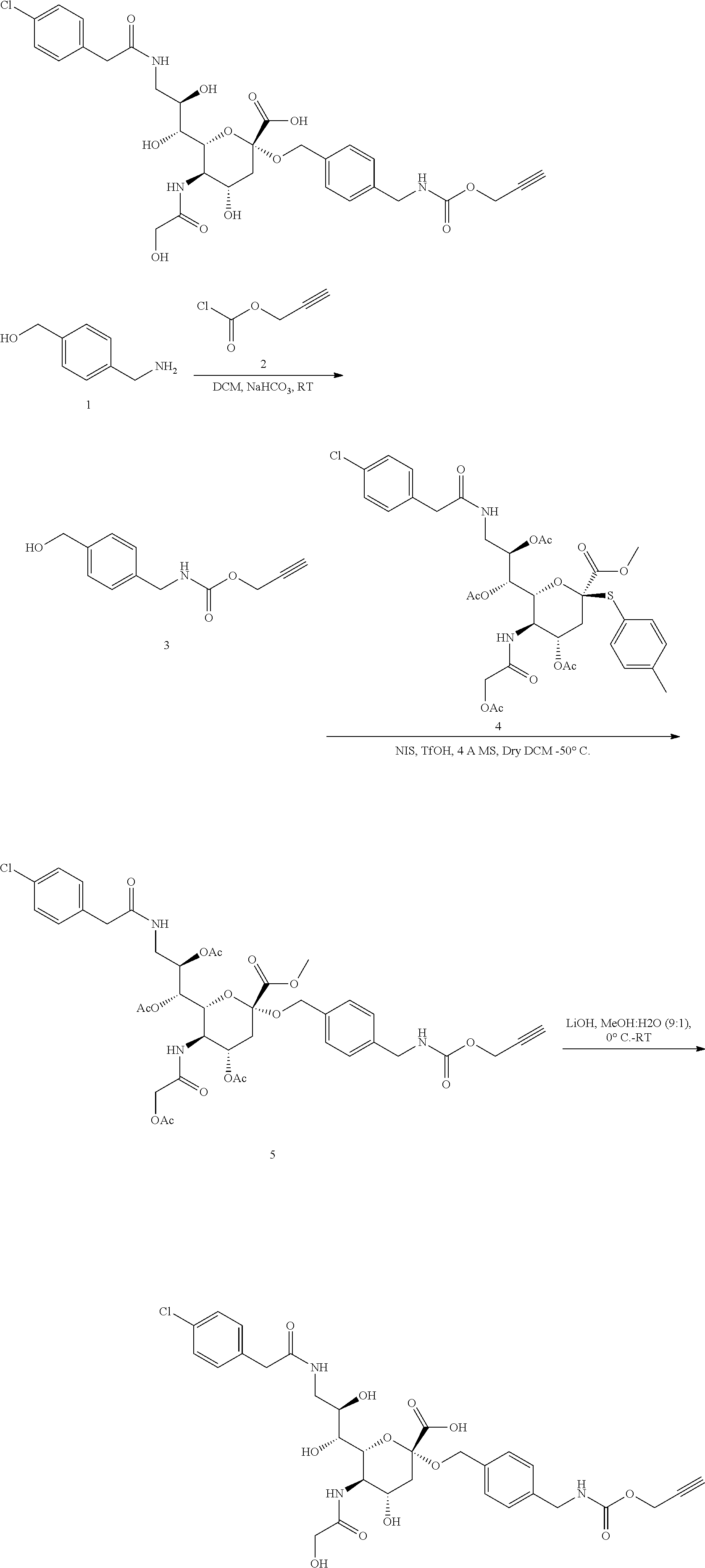

### Synthesis of prop-2-yn-1-yl (4-(hydroxymethyl)benzyl)carbamate (3)

To a stirred solution of (4-(aminomethyl)phenyl)methanol (1, 2.0 g, 14.6 mmol) and Prop-2-yn-1-yl carbonochloridate (2, 2.13 mL, 21.9 mmol) in diethyl ether (10 mL), sodium hydrogen carbonate (2.45 g, 29.2 mmol) were added and the reaction mixture were stirred at room temperature for 18 hrs. The reaction mixture was filtered through sintered funnel and washed with dichloromethane. Filtrate was washed by saturated solution of brine and dried over sodium sulfate, filtered and concentrated under reduced pressure to get crude compound. The crude product was purified by column chromatography using 12.0 g column and desired compound was eluted with 20-30% ethyl acetate in heptane to afford the prop-2-yn-1-yl (4-(hydroxymethyl)benzyl)carbamate (3) as a white solid. Yield: 2.0 g, 54%; ¹H NMR (400 MHz, Methanol-d₄): δ 7.89 (t, J=6.2 Hz, 1H), 7.26 (d, J=8.0 Hz, 2H), 7.19 (d, J=8.0 Hz, 2H), 4.63 (d, J=2.4 Hz, 2H), 4.46 (s, 2H), 4.16 (d, J=6.0 Hz, 2H), 3.50-3.48 (m, 1H).

### Synthesis of (1R,2R)-1-((2R,3R,4S,6R)-4-acetoxy-3-(2-acetoxyacetamido)-6-(methoxycarbonyl)-6-((4-((((prop-2-yn-1-yloxy)carbonyl)amino)methyl)benzyl)oxy)tetrahydro-2H-pyran-2-yl)-3-(2-(4-chlorophenyl)acetamido)propane-1,2-diyl diacetate (5)

A solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (4, 0.50 g, 0.65 mmol), prop-2-yn-1-yl (4-(hydroxymethyl)benzyl)carbamate (3, 0.86 g, 3.92 mmol) and activated 4 Å powdered molecular sieves (0.5 g) in anhydrous dichloromethane (10 mL) was stirred at room temperature for 15 h under nitrogen atmosphere. 1-iodopyrrolidine-2,5-dione (0.368 g, 1.63 mmol) and trifluoromethanesulfonic acid (0.006 mL, 0.65 mmol) solution in dichloromethane (1.0 mL) was added at −50° C. and the reaction mixture was continued to stir at same temperature for 1 h. Progress of the reaction was monitored by TLC and LCMS. After completion reaction mixture was quenched by triethylamine (0.5 mL) and warmed to room temperature. The reaction mixture was filtered through sintered funnel and washed with dichloromethane. Filtrate was washed by saturated solution of sodium bicarbonate and dried over sodium sulfate, filtered and concentrated under reduced pressure to obtain crude compound. The crude product was purified by flash column chromatography using 0-10% methanol in dichloromethane as eluent to afford (1R,2R)-1-((2R,3R,4S,6R)-4-acetoxy-3-(2-acetoxyacetamido)-6-(methoxycarbonyl)-6-((4-((((prop-2-yn-1-yloxy)carbonyl)amino)methyl)benzyl)oxy)tetrahydro-2H-pyran-2-yl)-3-(2-(4-chlorophenyl)acetamido)propane-1,2-diyl diacetate (5) as a brown fluffy solid with 66% purity along with beta isomer. Yield: 0.30 g, 54%; LCMS m/z 859.95 [M−1]⁻.

### Synthesis of (2R,4S,5R,6R)-2-(3-carboxypropoxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Compound 117)

To a stirred solution of (1R,2R)-1-((2R,3R,4S,6R)-4-acetoxy-3-(2-acetoxyacetamido)-6-(methoxycarbonyl)-6-((4-((((prop-2-yn-1-yloxy)carbonyl)amino)methyl)benzyl)oxy)tetrahydro-2H-pyran-2-yl)-3-(2-(4-chlorophenyl)acetamido)propane-1,2-diyl diacetate (5, 0.25 g, 0.29 mmol) in methanol (5 mL), was added solution of lithium hydroxide monohydrate (0.041 g, 1.74 mmol) in water (0.5 mL). The reaction mixture was stirred at room temperature for 6 h. The progress of the reaction was monitored by LCMS. After completion the reaction mixture was treated with acidic (Dowex 50, H⁺) to pH˜6 and the suspension was filtered through sintered funnel with methanol washing. The filtrate was concentrated under reduced pressure to get crude. The crude material was purified by prep HPLC using 0.1% TFA in water/Acetonitrile (100%) to afford ((2R,4S,5R,6R)-2-(3-carboxypropoxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Compound 117) as an off white solid. Yield: 0.034 g, 22%; LCMS m/z 678.3 [M+H]+; ¹H NMR (400 MHz, Methanol-d₄): δ 7.92 (d, J=8.0 Hz, 1H), 7.30-7.24 (m, 4H), 4.81 (d, J=12.0 Hz, 1H), 4.66 (d, J=2.4 Hz, 2H), 4.47 (d, J=11.2 Hz, 1H), 4.28 (s, 2H), 4.02 (s, 2H), 3.94-3.76 (m, 4H), 3.6 (dd, J=14.0 Hz & 3.2 Hz, 1H), 3.52 (s, 2H), 3.41 (dd, J=8.8 Hz & 1.6 Hz, 1H), 3.31-3.30 (m, 1H), 2.88 (t, J=2.4 Hz, 1H), 2.77 (dd, J=12.4 Hz & 4.4 Hz, 1H), 1.79 (t, J=12.0 Hz, 1H).

### (2S,4S,5R,6R)-6-((1R,2R)-3-(2-(4-chlorophenyl)acetamido)-1,2-dihydroxypropyl)-4-hydroxy-5-(2-hydroxyacetamido)-2-((4-(prop-2-yn-1-ylcarbamoyl)benzyl)thio)tetrahydro-2H-pyran-2-carboxylic acid (Compound 187)

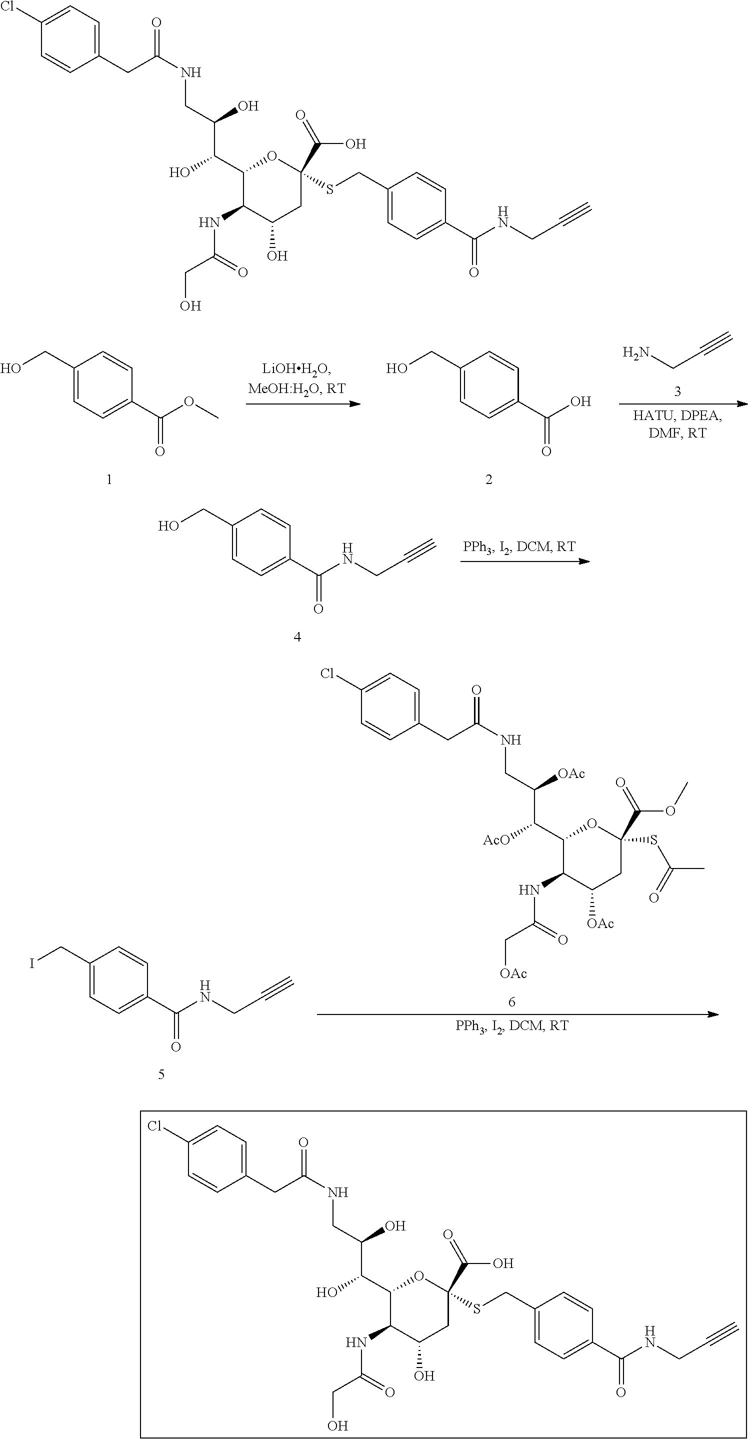

### Synthesis of 4-(hydroxymethyl)benzoic acid (2)

To a stirred solution of methyl 4-(hydroxymethyl)benzoate (1, 1.0 g, 6.02 mmol) in methanol (8.0 mL) was added solution of lithium hydroxide monohydrate (0.505 g, 12.04 mmol) in water (2.0 mL). The reaction mixture was stirred at room temperature for 5 h. Progress of the reaction was monitored by TLC. After completion the reaction mixture was concentrated under reduced pressure and the mixture was treated with 1N HCl solution and the suspension was filtered through sintered funnel and washed with water and pentane to afford 4-(hydroxymethyl)benzoic acid (2) as off white solid. Yield: 0.70 g, 76%; LCMS (ESI) m/z 151.05 [M−1]⁻.

### Synthesis of 4-(hydroxymethyl)-N-(prop-2-yn-1-yl)benzamide (4)

To a stirred solution of 4-(hydroxymethyl)benzoic acid (2, 0.50 g, 3.29 mmol) in N, N-Dimethylformamide (5.0 mL) 1,1,3,3-tetramethyl-2-(3H-1,2,3,4-tetraazainden-3-yl)-3-isoureaium hexafluoridophosphate(1-) (2.50 g, 6.57 mmol), ethylbis(propan-2-yl)amine (1.72 mL, 9.86 mmol) and prop-2-yn-1-amine (3, 0.362 g, 6.57 mmol) were added at 0° C. and reaction mixture was stirred at room temperature for 3 h. After completion the reaction mixture was diluted with water and ethyl acetate, organic layer was separated, dried over sodium sulphate, filtered and concentrated under reduced pressure to get crude. The crude was purified by column chromatography by using 12 g column and desired compound was eluted with 20-25% ethyl acetate in heptane to afford 4-(hydroxymethyl)-N-(prop-2-yn-1-yl)benzamide (4) as off white solid. Yield: 0.35 g, 56%; LCMS (ESI) m/z 190.10 [M+H]+.

### Synthesis of 4-(iodomethyl)-N-(prop-2-yn-1-yl)benzamide (5)

To a stirred solution of 4-(hydroxymethyl)benzoic acid (4, 0.35 g, 1.85 mmol) in dichloromethane (5.0 mL), triphenylphosphane (0.582 g, 2.22 mmol) and iodine (0.563 g, 2.22 mmol) was added at 0° C. and reaction mixture was stirred at room temperature for 3 h. After completion the reaction mixture was diluted with water and dichloromethane, organic layer was separated and washed with sodium metabisulphate solution. Organic layer was dried over sodium sulphate, filtered and concentrated under reduced pressure to get crude. The crude was purified by column chromatography by using 12 g column and desired compound was eluted with 20-25% ethyl acetate in heptane to afford 4-(iodomethyl)-N-(prop-2-yn-1-yl)benzamide (5) as an off white solid. Yield: 0.220 g, 40%; LCMS (ESI) m/z 299.90 [M+H]+.

### Synthesis of (2S,4S,5R,6R)-6-((1R,2R)-3-(2-(4-chlorophenyl)acetamido)-1,2-dihydroxypropyl)-4-hydroxy-5-(2-hydroxyacetamido)-2-((4-(prop-2-yn-1-ylcarbamoyl)benzyl)thio)tetrahydro-2H-pyran-2-carboxylic acid (Compound 187)

ssTo a stirred solution of (1R,2R)-1-((2R,3R,4S,6S)-4-acetoxy-3-(2-acetoxyacetamido)-6-(acetylthio)-6-(methoxycarbonyl)tetrahydro-2H-pyran-2-yl)-3-(2-(4-chlorophenyl)acetamido)propane-1,2-diyl diacetate (6, 0.20 g 0.278 mmol) in methanol (3.0 mL) was added sodium thiomethoxide solution (0.028 g, 0.335 mmol) slowly at 0° C. The reaction mixture was stirred at 0° C. for 1 hr. Then added 4-(iodomethyl)-N-(prop-2-yn-1-yl)benzamide (5, 0.166 g, 0.557 mmol) at 0° C. and reaction mixture was stirred at room temperature for 1 hr. Progress of the reaction was monitored by LCMS. After completion, to the same reaction mixture was added lithium hydroxide (0.070 g, 1.67 mmol) and stirred at room temperature for 6 hr. The progress of the reaction was monitored by LCMS and after completion, Dowex-hydrogen form was added to made pH 6 and reaction mass was filtered through celite bed. The filtrate was concentrated under reduced pressure to obtained thick residue which was purified by preparative HPLC (15-40% acetonitrile in water with 0.1% TFA). Fractions containing the desired product were combined and lyophilized to dryness to afford (2S,4S,5R,6R)-6-((1R,2R)-3-(2-(4-chlorophenyl)acetamido)-1,2-dihydroxypropyl)-4-hydroxy-5-(2-hydroxyacetamido)-2-((4-(prop-2-yn-1-ylcarbamoyl)benzyl)thio)tetrahydro-2H-pyran-2-carboxylic acid (Compound 187) as a white color solid. Yield: 0.021 g, 11%; LCMS m/z 664.30 [M+H]+. ¹H NMR (400 MHz, MeOD-d4): δ 8.01-7.99 (m, 1H), 7.93 (d, J=8.0 Hz, 1H), 7.74 (d, J=8.0 Hz, 2H), 7.43 (d, J=8.0 Hz, 2H), 7.30-7.21 (m, 4H), 4.14 (d, J=2.4 Hz, 2H), 4.06-4.01 (m, 3H), 3.92-3.80 (m, 3H), 3.70 (td, J=8.4 Hz & 3.2 Hz, 1H), 3.64-3.52 (m, 2H), 3.48 (s, 2H), 3.35-3.33 (m, 1H), 3.23-3.17 (m, 1H), 2.79 (dd, J=12.8 Hz & 4.4 Hz, 1H), 2.59 (t, J=2.4 Hz, 1H), 1.79 (t, J=12.4 Hz, 1H).

### (2S,4S,5R,6R)-2-((4-carboxybenzyl)thio)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Compound 195)

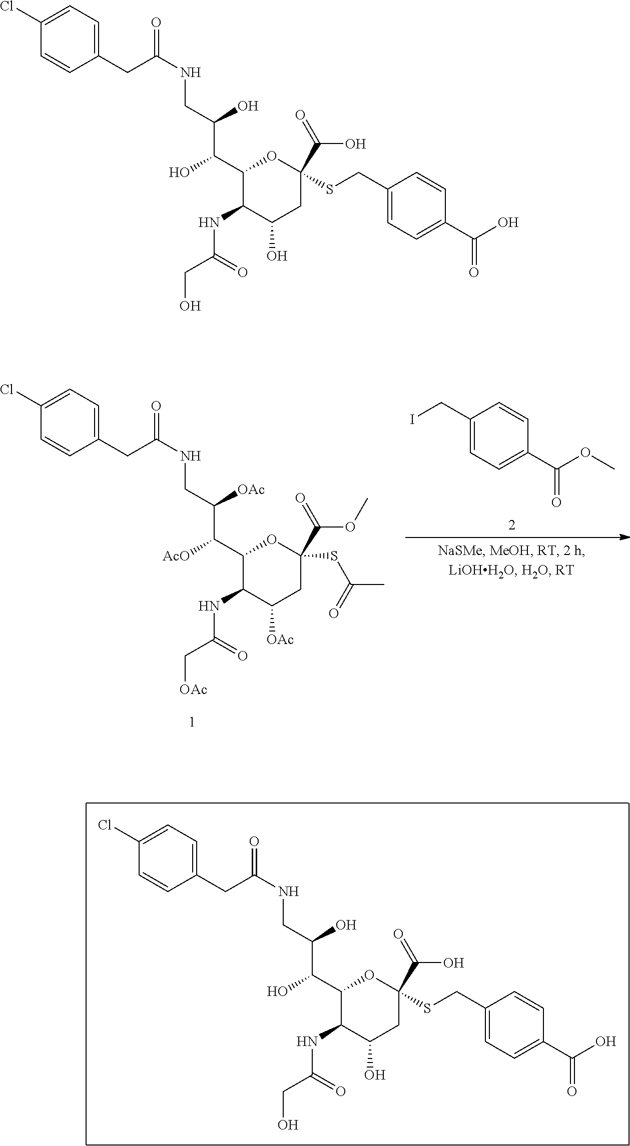 [SMILES: O=C(Cc1ccc(Cl)cc1)NC[C@@H](O)[C@@H](O)[C@@H]1O[C@@](SCc2ccc(C(=O)O)cc2)(C(=O)O)C[C@H](O)[C@H]1NC(=O)CO ; COC(=O)[C@@]1(SC(C)=O)C[C@H](OC(C)=O)[C@@H](NC(=O)COC(C)=O)[C@H]([C@H](OC(C)=O)[C@@H](CNC(=O)Cc2ccc(Cl)cc2)OC(C)=O)O1 ; COC(=O)c1ccc(CI)cc1 ; O=C(Cc1ccc(Cl)cc1)NC[C@@H](O)[C@@H](O)[C@@H]1O[C@@](SCc2ccc(C(=O)O)cc2)(C(=O)O)C[C@H](O)[C@H]1NC(=O)CO]

### Synthesis of (2S,4S,5R,6R)-2-((4-carboxybenzyl)thio)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Compound 195)

To a stirred solution (1R,2R)-1-((2R,3R,4S,6S)-4-acetoxy-3-(2-acetoxyacetamido)-6-(acetylthio)-6-(methoxycarbonyl)tetrahydro-2H-pyran-2-yl)-3-(2-(4-chlorophenyl)acetamido)propane-1,2-diyl diacetate (1, 0.20 g 0.278 mmol) in methanol (3.0 mL) was added sodium thiomethoxide solution (0.028 g, 0.335 mmol) slowly at 0° C. The reaction mixture was stirred at 0° C. for 1 hr. To the reaction mixture was added methyl 4-(iodomethyl)benzoate (2, 0.153 g, 0.557 mmol) at 0° C. and reaction mixture was stirred at room temperature for 1 hr. Progress of the reaction was monitored by LCMS. After completion, to the same reaction mass was added lithium hydroxide (0.071 g, 1.66 mmol) at room temperature and the reaction was stirred at room temperature for 6 h. The progress of the reaction was monitored by LCMS and after completion Dowex-hydrogen form was added to made pH 6 and reaction mass was filtered through celite bed. The filtrate was concentrated under reduced pressure to obtained thick residue which was purified by preparative HPLC (15-40% acetonitrile in water with 0.1% TFA). Fractions containing the desired product were combined and lyophilized to dryness to afford (2S,4S,5R,6R)-2-((4-carboxybenzyl)thio)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Compound 195) as a white color solid. Yield: 0.011 g, 7.0%; LCMS m/z 627.30 [M+H]+. ¹H NMR (400 MHz, MeOD-d4): δ 7.95 (d, J=8.0 Hz, 2H), 7.43 (d, J=8.0 Hz, 2H), 7.27-7.21 (m, 4H), 4.07 (d, J=12.8 Hz, 1H), 4.01 (s, 2H), 3.93 (d, J=12.8 Hz, 1H), 3.91-3.74 (m, 3H), 3.65 -3.60 (m, 2H), 3.52 (s, 2H), 3.35 (dd, J=8.8 Hz & 1.6 Hz, 1H), 3.23-3.17 (m, 1H), 2.82 (dd, 12.4 Hz & 3.6 Hz, 1H), 1.77 (t, J=12.0 Hz, 1H).

### (2S,4S,5R,6R)-2-((3-carboxybenzyl)thio)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Compound 116)

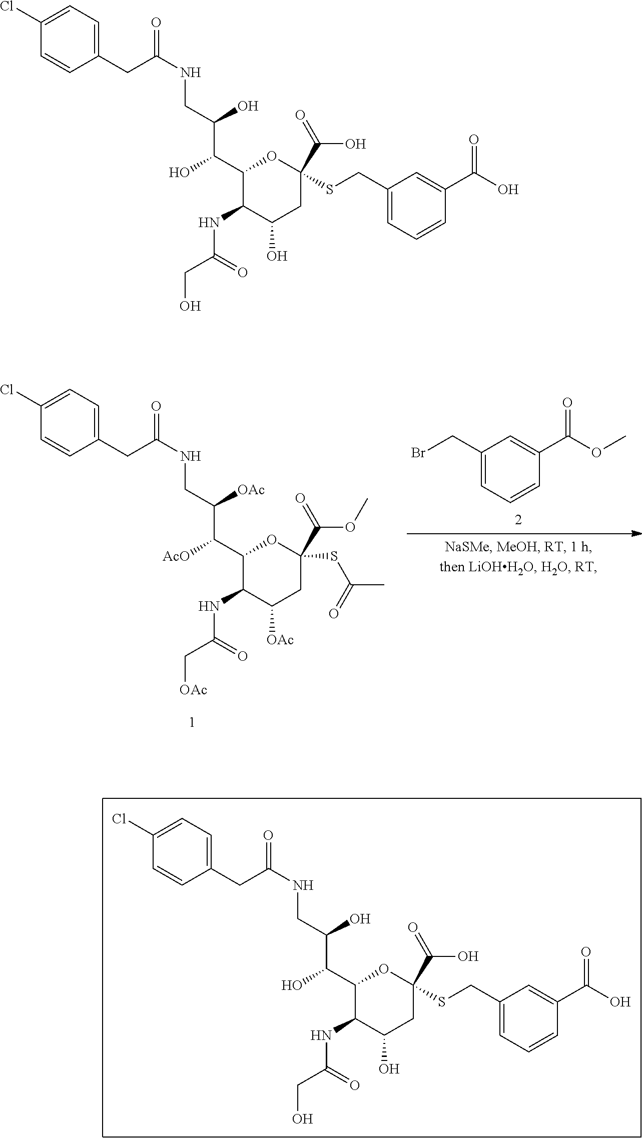 [SMILES: O=C(Cc1ccc(Cl)cc1)NC[C@@H](O)[C@@H](O)[C@@H]1O[C@@](SCc2cccc(C(=O)O)c2)(C(=O)O)C[C@H](O)[C@H]1NC(=O)CO ; COC(=O)[C@@]1(SC(C)=O)C[C@H](OC(C)=O)[C@@H](NC(=O)COC(C)=O)[C@H]([C@H](OC(C)=O)[C@@H](CNC(=O)Cc2ccc(Cl)cc2)OC(C)=O)O1 ; COC(=O)c1cccc(CBr)c1 ; O=C(Cc1ccc(Cl)cc1)NC[C@@H](O)[C@@H](O)[C@@H]1O[C@@](SCc2cccc(C(=O)O)c2)(C(=O)O)C[C@H](O)[C@H]1NC(=O)CO]

### Synthesis of (2S,4S,5R,6R)-2-((3-carboxybenzyl)thio)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Compound 116)

ssTo a stirred solution of (1R,2R)-1-((2R,3R,4S,6S)-4-acetoxy-3-(2-acetoxyacetamido)-6-(acetylthio)-6-(methoxycarbonyl)tetrahydro-2H-pyran-2-yl)-3-(2-(4-chlorophenyl)acetamido)propane-1,2-diyl diacetate (1, 0.20 g, 0.278 mmol) in methanol (2.0 ml) was added sodium thiomethoxide (0.028 g, 0.333 mmol) at 0° C. and stirred for 1 h. Then added methyl 3-(bromomethyl)benzoate (2, 0.127 g, 0.557 mmol) at 0° C. and reaction mixture was stirred at room temperature for 1 hr. Progress of the reaction was monitored by LCMS. After completion, to the same reaction mass was added lithium hydroxide (0.071 g, 1.66 mmol) at room temperature and the reaction was stirred at room temperature for 6 hr. The progress of the reaction was monitored by LCMS and after completion, Dowex-hydrogen form was added to made pH˜6 and reaction mixture was filtered through celite bed. The filtrate was concentrated under reduced pressure to obtained thick residue which was purified by preparative HPLC (15-40% acetonitrile in water with 0.1% TFA). Fractions containing the desired product were combined and lyophilized to dryness to afford (2S,4S,5R,6R)-2-((3-carboxybenzyl)thio)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Compound 116) as a white solid. Yield: 0.115 g; 66%; LCMS (ESI) m/z 627.25 [M+H]+. ¹H NMR (400 MHz, MeOD-d4) δ 7.97 (s, 1H), 7.89 (d, J=7.6 Hz, 1H), 7.57 (d, J=7.6 Hz, 1H), 7.40 (t, J=7.6 Hz, 1H), 7.27-7.21 (m, 4H), 4.07 (d, J=12.8 Hz, 1H), 4.01 (s, 2H), 3.92 (d, J=12.4 Hz, 1H), 3.89-3.82 (m, 2H), 3.81-3.77 (m, 1H), 3.67-3.61 (m, 2H), 3.52 (s, 2H), 3.31 (dd, J=13.6 Hz & 2.8 Hz, 1H), 3.24-3.18 (m, 1H), 2.80 (dd, J=12.4 Hz & 4.0 Hz, 1H), 1.80 (t, J=12.4 Hz, 1H).

### (2R,4S,5R,6R)-6-((1R,2R)-3-(2-(4-chlorophenyl)acetamido)-1,2-dihydroxypropyl)-4-hydroxy-2-(4-((hydroxy(prop-2-yn-1-yloxy)phosphoryl)oxy)butoxy)-5-(2-hydroxyacetamido)tetrahydro-2H-pyran-2-carboxylic acid (Compound 115)

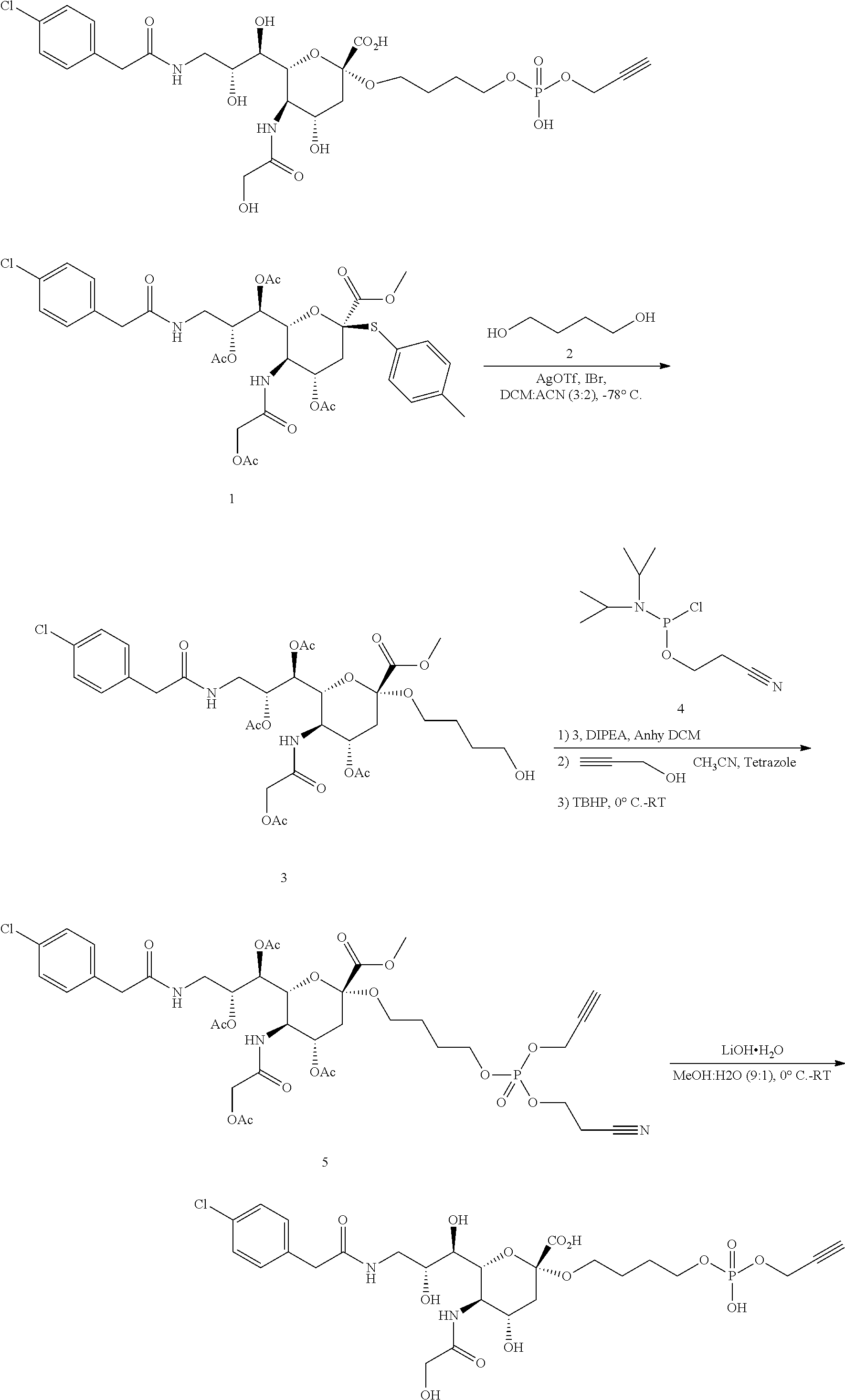

### Synthesis of (1R,2R)-1-((2R,3R,4S,6R)-4-acetoxy-3-(2-acetoxyacetamido)-6-(4-hydroxybutoxy)-6-(methoxycarbonyl)tetrahydro-2H-pyran-2-yl)-3-(2-(4-chlorophenyl)acetamido)propane-1,2-diyl diacetate (3)

A solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (1, 0.20 g, 0.26 mmol), butane-1,4-diol (2, 0.12 g, 1.30 mmol), silver trifluoromethanesulfonate (0.13 g, 0.52 mmol) and activated 4 Å powdered molecular sieves (0.5 g) in anhydrous dichloromethane (18.0 mL) and acetonitrile (30.0 mL) was stirred at room temperature for 2 h under nitrogen atmosphere. Iodine mono bromide (0.21 g, 1.05 mmol) solution in dichloromethane (2.0 mL) was added at −78° C. and the reaction mixture was stirred at the same temperature for 2 h. Progress of the reaction was monitored by TLC (100% EA) and LCMS. After completion, the reaction mixture was quenched by triethyl amine (0.1 mL) and warmed to room temperature. The reaction mixture was filtered through celite bed and washed by dichloromethane. Filtrate was washed with saturated solution of sodium thiosulphate and dried over sodium sulfate, filtered and concentrated under reduced pressure to get crude compound. The crude product was purified by column chromatography using 12.0 g column and desired compound was eluted with 80-100% ethyl acetate in hexane to afford methyl (1R,2R)-1-((2R,3R,4S,6R)-4-acetoxy-3-(2-acetoxyacetamido)-6-(4-hydroxybutoxy)-6-(methoxycarbonyl)tetrahydro-2H-pyran-2-yl)-3-(2-(4-chlorophenyl)acetamido)propane-1,2-diyl diacetate (3) as a fluffy solid. Yield: 0.1 g, 52%; LCMS (ESI) m/z 731.0 [M+H]+.

### Synthesis of (1R,2R)-1-((2R,3R,4S,6R)-4-acetoxy-3-(2-acetoxyacetamido)-6-(4-(((2-cyanoethoxy)(prop-2-yn-1-yloxy)phosphoryl)oxy)butoxy)-6-(methoxycarbonyl)tetrahydro-2H-pyran-2-yl)-3-(2-(4-chlorophenyl)acetamido)propane-1,2-diyl diacetate (5)

To the solution of 3-((chloro(diisopropylamino)phosphaneyl)oxy)propanenitrile (4, 0.097 g, 0.41 mmol) in dichloromethane (4 ml), ethylbis(propan-2-yl)amine (0.11 mL, 0.615 mmol) was added dropwise at 0° C. and the reaction mixture was stirred at same temperature for 10 minutes, then methyl (1R,2R)-1-((2R,3R,4S,6R)-4-acetoxy-3-(2-acetoxyacetamido)-6-(4-hydroxybutoxy)-6-(methoxycarbonyl)tetrahydro-2H-pyran-2-yl)-3-(2-(4-chlorophenyl)acetamido)propane-1,2-diyl diacetate (3, 0.15 g, 0.205 mmol) in dichloromethane (1 mL) was added to the reaction mixture and the reaction mixture was stirred for another 30 minutes at room temperature. The progress of the reaction was monitored by TLC (100% EA). After completion propargyl alcohol (0.024 mL, 0.41 mmol) and tetrazole (0.028 g, 0.41 mmol) in acetonitrile (0.5 mL) were added sequentially at 0° C. and the reaction mixture was stirred for another 1 h at room temperature. The progress of the reaction was monitored by LCMS. After completion, TBHP (0.07 mL) was added at 0° C. and the reaction mixture was stirred at room temperature for 1 h. Progress of the reaction mixture was monitored by LCMS. After completion, reaction mixture was directly concentrated under reduced pressure to afford crude (1R,2R)-1-((2R,3R,4S,6R)-4-acetoxy-3-(2-acetoxyacetamido)-6-(4-(((2-cyanoethoxy)(prop-2-yn-1-yloxy)phosphoryl)oxy)butoxy)-6-(methoxycarbonyl)tetrahydro-2H-pyran-2-yl)-3-(2-(4-chlorophenyl)acetamido)propane-1,2-diyl diacetate (5) as a deep brown viscous syrup which was directly used for the next step without further purification. Yield: 0.23 g, crude; LCMS m/z 902.05 [M+H]+.

### Synthesis of (2R,4S,5R,6R)-6-((1R,2R)-3-(2-(4-chlorophenyl)acetamido)-1,2-dihydroxypropyl)-4-hydroxy-2-(4-((hydroxy(prop-2-yn-1-yloxy)phosphoryl)oxy)butoxy)-5-(2-hydroxyacetamido)tetrahydro-2H-pyran-2-carboxylic acid (Compound 115)

ssTo the stirred solution of (1R,2R)-1-((2R,3R,4S,6R)-4-acetoxy-3-(2-acetoxyacetamido)-6-(4-(((2-cyanoethoxy)(prop-2-yn-1-yloxy)phosphoryl)oxy)butoxy)-6-(methoxycarbonyl)tetrahydro-2H-pyran-2-yl)-3-(2-(4-chlorophenyl)acetamido)propane-1,2-diyl diacetate (5, 0.15 g, 0.166 mmol) in methanol (5.0 mL) and water (0.5 mL), lithium hydroxide monohydrate (0.023 g, 0.998 mmol) was added at 0° C. The reaction mixture was stirred at room temperature for 3 h. Progress of the reaction was monitored by LCMS. After completion, the reaction mixture was quenched with Dowex-H⁺ resin and filtered with methanol (2-3 times). The organic layer was concentrated under reduced pressure to afford crude residue. The crude was purified by prep-HPLC using 0-100% acetonitrile/water as eluent and the desired fractions were lyophilized to afford (2R,4S,5R,6R)-6-((1R,2R)-3-(2-(4-chlorophenyl)acetamido)-1,2-dihydroxypropyl)-4-hydroxy-2-(4-((hydroxy(prop-2-yn-1-yloxy)phosphoryl)oxy)butoxy)-5-(2-hydroxyacetamido)tetrahydro-2H-pyran-2-carboxylic acid (Compound 115) as an off-white solid; Yield: 0.016 g, 14%; LCMS m/z 667.25 [M+H]+; ¹H-NMR (400 MHz, Methanol-d4): δ 7.32-7.28 (m, 4H), 4.60 (dd, J=10.0 Hz & 2.4 Hz, 1H), 4.05-4.01 (m, 4H), 3.92-3.70 (m, 5H), 3.61 (dd, J=14.0 Hz & 2.8 Hz, 1H), 3.54 (s, 2H), 3.48-3.43 (m, 1H), 3.38-3.35 (m, 1H), 3.30-3.23 (m, 1H), 3.01 (t, J=3.0 Hz, 1H), 2.71 (dd, J=12.4 Hz & 4.0 Hz, 1H), 1.77-1.65 (m, 5H).

### (2R,4S,5R,6R)-6-((1R,2R)-3-(2-(4-chlorophenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Compound 113)

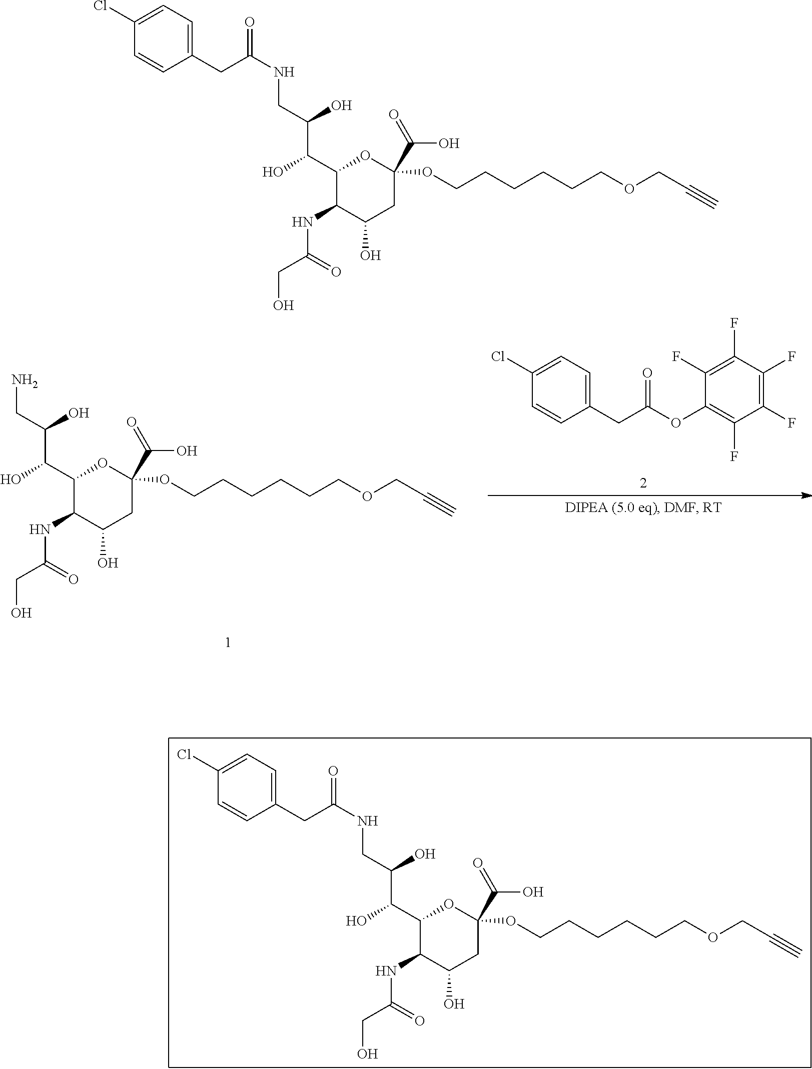 [SMILES: C#CCOCCCCCCO[C@]1(C(=O)O)C[C@H](O)[C@@H](NC(=O)CO)[C@H]([C@H](O)[C@H](O)CNC(=O)Cc2ccc(Cl)cc2)O1 ; C#CCOCCCCCCO[C@]1(C(=O)O)C[C@H](O)[C@@H](NC(=O)CO)[C@H]([C@H](O)[C@H](O)CN)O1 ; O=C(Cc1ccc(Cl)cc1)Oc1c(F)c(F)c(F)c(F)c1F ; C#CCOCCCCCCO[C@]1(C(=O)O)C[C@H](O)[C@@H](NC(=O)CO)[C@H]([C@H](O)[C@H](O)CNC(=O)Cc2ccc(Cl)cc2)O1]

### Synthesis of (2R,4S,5R,6R)-6-((1R,2R)-3-(2-(4-chlorophenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Compound 113)

ssTo a stirred solution of (2R,4S,5R,6R)-6-((1R,2R)-3-amino-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (1, 0.20 g, 0.432 mmol) and perfluorophenyl 2-(4-chlorophenyl)acetate (2, 0.146 g, 0.432 mmol) in N,N-dimethylformamide (4.0 mL) was added ethylbis(propan-2-yl)amine (0.37 mL, 2.16 mmol) at 0° C. and the reaction mixture was stirred at room temperature for 4 h. The reaction mixture was concentrated and the crude residue obtained was purified by prep HPLC using acetonitrile and water (+0.1% TFA) as eluent to afford the desired product. Yield: 0.032 g, 12%; LCMS m/z 615.45 [M+H]+; 1H NMR (400 MHz, methanol-d4) 8.02-7.99 (m, 1H), 7.88 (d, J=8.0 Hz, 1H), 7.31-7.27 (m, 4H), 4.12 (d, J=2.4 Hz, 2H), 4.01 (s, 2H), 3.89-3.62 (m, 6H), 3.52-3.43 (m, 4H), 3.43-3.34 (m, 2H), 3.26-3.21 (m, 1H), 2.80 (t, J=2.4 Hz, 1H), 2.72 (dd, J=4.0 Hz & 12.4 Hz, 1H), 1.71 (t, J=12.4 Hz, 1H), 1.57-1.53 (m, 4H), 1.38-1.35 (m, 4H).

### (2R,4S,5R,6R)-2-(4-((1-carboxybut-3-yn-1-yl)amino)-4-oxobutoxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Compound 102)

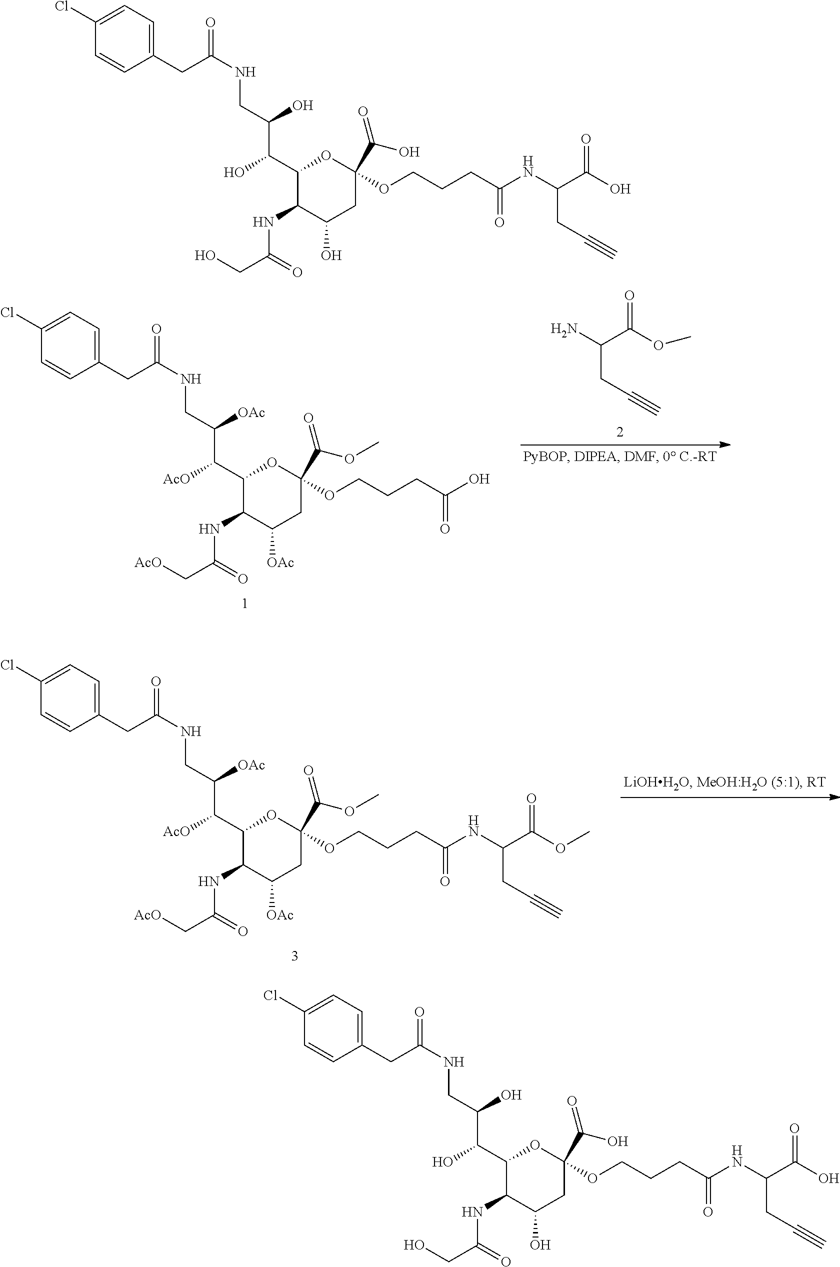

### Synthesis of (1R,2R)-1-((2R,3R,4S,6R)-4-acetoxy-3-(2-acetoxyacetamido)-6-(4-((1-methoxy-1-oxopent-4-yn-2-yl)amino)-4-oxobutoxy)-6-(methoxycarbonyl)tetrahydro-2H-pyran-2-yl)-3-(2-(4-chlorophenyl)acetamido)propane-1,2-diyl diacetate (3)

To a stirred solution of 4-(((2R,4S,5R,6R)-4-acetoxy-5-(2-acetoxyacetamido)-6-((1R,2R)-1,2-diacetoxy-3-(2-(4-chlorophenyl)acetamido)propyl)-2-(methoxycarbonyl)tetrahydro-2H-pyran-2-yl)oxy)butanoic acid (1, 0.5 g, 0.671 mmol) and methyl 2-amino-4-pentynoate (2, 0.128 g, 1.01 mmol) in N,N dimethylformamide (5 mL) were added 1H-1,2,3-benzotriazol-1-yloxy-tri-1-pyrrolidinylphosphonium hexafluoridophosphate(1-) (0.698 g, 1.34 mmol) and ethylbis(propan-2-yl)amine (0.7 mL, 4.03 mmol) sequentially at 0° C. and reaction mixture was stirred at room temperature for 6 hrs. Progress of the reaction was monitored by LCMS. After completion, reaction mixture was concentrated under reduced pressure to obtain crude residue which was purified by flash column chromatography using 0-10% methanol in dichloromethane as eluent to afford (1R,2R)-1-((2R,3R,4S,6R)-4-acetoxy-3-(2-acetoxyacetamido)-6-(4-((1-methoxy-1-oxopent-4-yn-2-yl)amino)-4-oxobutoxy)-6-(methoxycarbonyl)tetrahydro-2H-pyran-2-yl)-3-(2-(4-chlorophenyl)acetamido)propane-1,2-diyl diacetate (3) as a colorless liquid. Yield: 0.35 g, 61%; LCMS m/z 853.95 [M+H]+.

### Synthesis of (2R,4S,5R,6R)-2-(4-((I-carboxybut-3-yn-1-yl)amino)-4-oxobutoxy)-6-((R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Compound 102)

ssTo a stirred solution of (1R,2R)-1-((2R,3R,4S,6R)-4-acetoxy-3-(2-acetoxyacetamido)-6-(4-((1-methoxy-1-oxopent-4-yn-2-yl)amino)-4-oxobutoxy)-6-(methoxycarbonyl)tetrahydro-2H-pyran-2-yl)-3-(2-(4-chlorophenyl)acetamido)propane-1,2-diyl diacetate (3, 0.35 g, 0.33 mmol) in methanol (5 mL) were added solution of lithium hydroxide monohydrate (0.047 g, 1.97 mmol) in water (0.5 mL). The reaction mixture was stirred at room temperature for 6 hrs. The progress of the reaction was monitored by LCMS. After completion the reaction mixture was treated with acidic (Dowex 50, H⁺) to pH˜6 and the suspension was filtered through sintered funnel with methanol washing. The filtrate was concentrated under reduced pressure to get crude. The crude material was purified by prep HPLC using water/Acetonitrile solvent system with 0.1% TFA as additive to afford (2R,4S,5R,6R)-2-(4-((1-carboxybut-3-yn-1-yl)amino)-4-oxobutoxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Compound 102) as an off white solid. Yield: 0.061 g, 22%; LCMS m/z 658.40 [M+H]+; ¹H NMR (400 MHz, Methanol-d₄) δ 7.90 (d, J=7.6 Hz, 1H), 7.32-7.27 (m, 4H), 4.54 (t, J=6.4 Hz, 1H), 4.01 (s, 2H), 3.92-3.78 (m, 4H), 3.71 (d, J=9.6 Hz, 1H), 3.66-3.59 (m, 1H), 3.53 (s, 2H), 3.49-3.44 (m, 1H), 3.38-3.36 (m, 1H), 2.77-2.64 (m, 4H), 2.37-2.33 (m, 3H), 1.90-1.83 (m, 2H), 1.75 (t, J=12.4 Hz, 1H).

### (2R,4S,5R,6R)-2-(4-((carboxymethyl)(prop-2-yn-1-yl)amino)-4-oxobutoxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Compound 101)

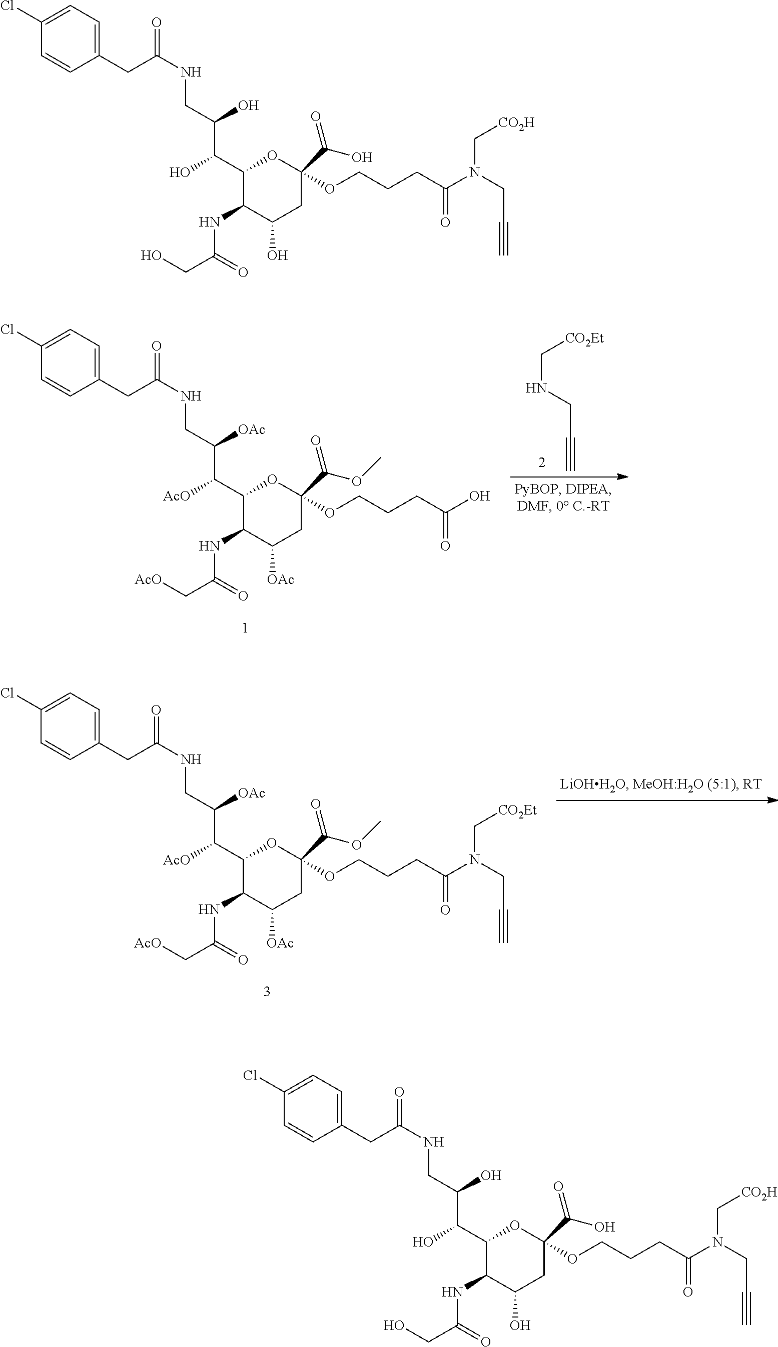

### Synthesis of (1R,2R)-1-((2R,3R,4S,6R)-4-acetoxy-3-(2-acetoxyacetamido)-6-(4-((2-ethoxy-2-oxoethyl)(prop-2-yn-1-yl)amino)-4-oxobutoxy)-6-(methoxycarbonyl)tetrahydro-2H-pyran-2-yl)-3-(2-(4-chlorophenyl)acetamido)propane-1,2-diyl diacetate (3)

To a stirred solution of 4-(((2R,4S,5R,6R)-4-acetoxy-5-(2-acetoxyacetamido)-6-((1R,2R)-1,2-diacetoxy-3-(2-(4-chlorophenyl)acetamido)propyl)-2-(methoxycarbonyl)tetrahydro-2H-pyran-2-yl)oxy)butanoic acid (1, 0.35 g, 0.31 mmol) and ethyl prop-2-yn-1-ylglycinate (2, 0.065 g, 0.46 mmol) in N,N dimethylformamide (5 mL) were added 1H-1,2,3-benzotriazol-1-yloxy-tri-1-pyrrolidinylphosphonium hexafluoridophosphate(1-) (0.32 g, 0.62 mmol) and ethylbis(propan-2-yl)amine (0.27 mL, 1.55 mmol) sequentially at 0° C. and reaction mixture was stirred at room temperature for 6 hrs. Progress of the reaction was monitored by LCMS. After completion reaction mixture was concentrated under reduced pressure to obtain crude residue which was purified by flash column chromatography using 0-10% methanol in dichloromethane as eluent to afford (1R,2R)-1-((2R,3R,4S,6R)-4-acetoxy-3-(2-acetoxyacetamido)-6-(4-((2-ethoxy-2-oxoethyl)(prop-2-yn-1-yl)amino)-4-oxobutoxy)-6-(methoxycarbonyl)tetrahydro-2H-pyran-2-yl)-3-(2-(4-chlorophenyl)acetamido)propane-1,2-diyl diacetate (3) as a colorless liquid. Yield: 0.25 g, 61%; LCMS m/z 868.95 [M+H]+.

### Synthesis of (2R,4S,5R,6R)-2-(4-((carboxymethyl)(prop-2-yn-1-yl)amino)-4-oxobutoxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Compound 101)

To a stirred solution of (1R,2R)-1-((2R,3R,4S,6R)-4-acetoxy-3-(2-acetoxyacetamido)-6-(4-((2-ethoxy-2-oxoethyl)(prop-2-yn-1-yl)amino)-4-oxobutoxy)-6-(methoxycarbonyl)tetrahydro-2H-pyran-2-yl)-3-(2-(4-chlorophenyl)acetamido)propane-1,2-diyl diacetate (3, 0.50 g, 0.57 mmol) in methanol (10 mL) was added solution of lithium hydroxide monohydrate (0.082 g, 3.46 mmol) in water (1.0 mL). The reaction mixture was stirred at room temperature for 6 hrs. The progress of the reaction was monitored by LCMS. After completion the reaction mixture was treated with acidic (Dowex 50, H⁺) to pH˜6 and the suspension was filtered through sintered funnel with methanol washing. The filtrate was concentrated under reduced pressure to obtain crude. The crude material was purified by prep HPLC using water/Acetonitrile solvent mixture and 0.1% TFA as additive to afford (2R,4S,5R,6R)-2-(4-((carboxymethyl)(prop-2-yn-1-yl)amino)-4-oxobutoxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Compound 101) as an off white solid. Yield: 0.127 g, 31%; LCMS m/z 658.35 [M+H]+; ¹H NMR (400 MHz, Methanol-d₄) δ 7.90 (d, J=6.0 Hz, 1H), 7.32-7.27 (m, 4H), 4.32 (s, 1H), 4.29 (t, J=6.4 Hz, 1H), 4.25 (dd, J=14.4 Hz & 2.8 Hz, 1H), 4.16 (s, 2H), 4.02 (s, 2H), 3.89-3.78 (m, 4H), 3.72 (d, J=9.6 Hz, 1H), 3.63 (dd, J=14.4 Hz & 2.8 Hz, 1H), 3.53 (s, 2H), 3.50-3.46 (m, 1H), 3.37-3.31 (m, 1H), 3.28-3.23 (m, 1H), 2.80 (t, J=2.4 Hz, 1H), 2.74-2.67 (m, 1H), 2.69 (t, J=2.4 Hz, 1H), 2.61 (t, J=6.8 Hz, 1H), 2.41 (t, J=7.2 Hz, 1H), 1.91-1.83 (m, 2H), 1.78-1.70 (m, 1H).

### (2R,4S,5R,6R)-2-((6-((carboxymethyl)(prop-2-yn-1-yl)amino)-6-oxohexyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Compound 100)

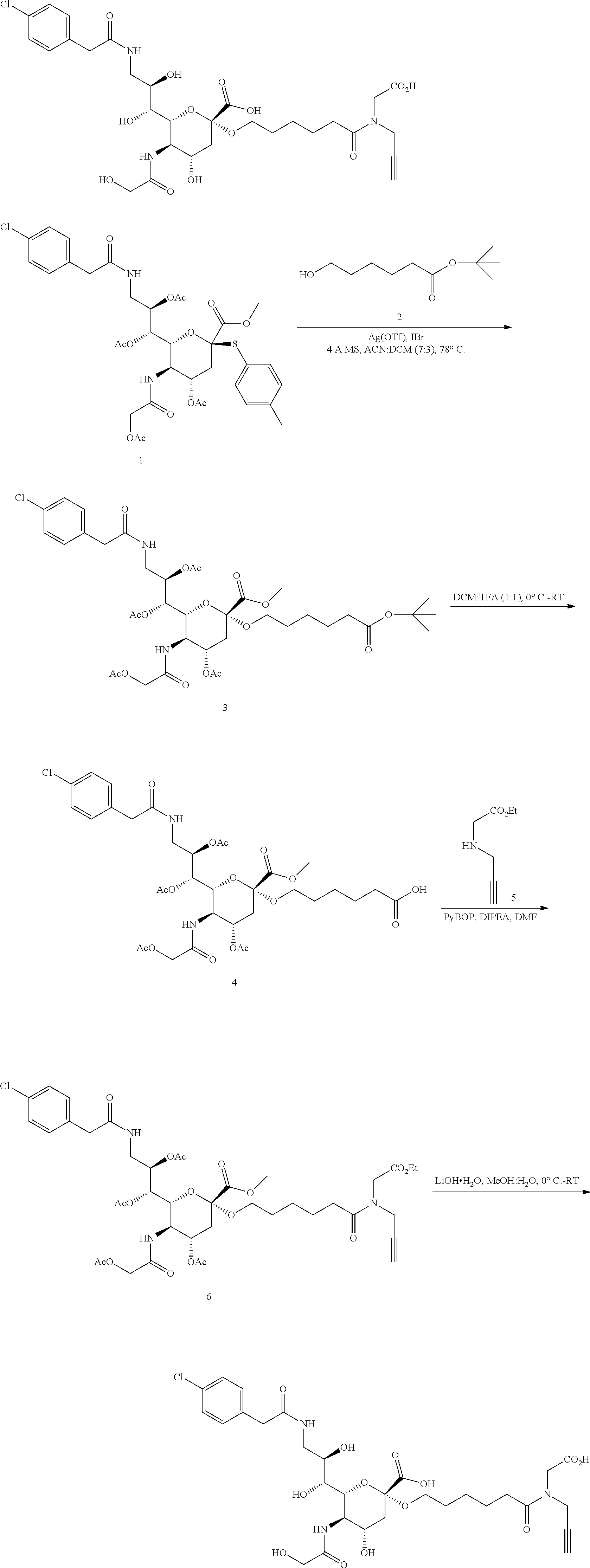

### Synthesis of (1R,2R)-1-((2R,3R,4S,6R)-4-acetoxy-3-(2-acetoxyacetamido)-6-((6-(tert-butoxy)-6-oxohexyl)oxy)-6-(methoxycarbonyl)tetrahydro-2H-pyran-2-yl)-3-(2-(4-chlorophenyl)acetamido)propane-1,2-diyl diacetate (3)

A solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (1, 0.50 g, 0.65 mmol), tert-butyl 6-hydroxyhexanoate (2, 0.61 g, 3.27 mmol), silver(1+) trifluoromethanesulfonate (0.50 g, 1.96 mmol) and activated 4 Å powdered molecular sieves (1.00 g) in solvent mixtures of anhydrous dichloromethane (35 mL) and anhydrous acetonitrile (60 mL) were stirred at room temperature for 1 h under nitrogen atmosphere. Thereafter, the solution was cooled to −78° C. and to it iodobromane (0.27 g, 1.31 mmol) solution in dichloromethane (5.0 mL) was added dropwise and was stirred at −78° C. for 2 h. The reaction was monitored by TLC. After completion, the reaction mixture was quenched by triethylamine (1.0 mL) and warmed to room temperature. The reaction mixture was filtered through sintered funnel and washed by dichloromethane. Filtrate was washed by saturated solution of sodium bicarbonate and dried over sodium sulfate, filtered and concentrated under reduced pressure to get crude. The crude product was purified by column chromatography using 60-70% ethyl acetate in heptane solvent mixtures as elutant which afforded (1R,2R)-1-((2R,3R,4S,6R)-4-acetoxy-3-(2-acetoxyacetamido)-6-((6-(tert-butoxy)-6-oxohexyl)oxy)-6-(methoxycarbonyl)tetrahydro-2H-pyran-2-yl)-3-(2-(4-chlorophenyl)acetamido)propane-1,2-diyl diacetate (3) as a white color solid. Yield: 0.40 g, 34%; LCMS m/z 827.10 [M−1]⁻.

### Synthesis of 6-(((2R,4S,5R,6R)-4-acetoxy-5-(2-acetoxyacetamido)-6-((1R,2R)-1,2-diacetoxy-3-(2-(4-chlorophenyl)acetamido)propyl)-2-(methoxycarbonyl)tetrahydro-2H-pyran-2-yl)oxy)hexanoic acid (4)

To a stirred solution of (1R,2R)-1-((2R,3R,4S,6R)-4-acetoxy-3-(2-acetoxyacetamido)-6-((6-(tert-butoxy)-6-oxohexyl)oxy)-6-(methoxycarbonyl)tetrahydro-2H-pyran-2-yl)-3-(2-(4-chlorophenyl)acetamido)propane-1,2-diyl diacetate (3, 0.40 g, 0.48 mmol), in anhydrous dichloromethane (4.0 mL), trifluoroacetic acid (2.0 mL) was added at 0° C. and the reaction mixture was stirred at room temperature for 3 h. Progress of the reaction was monitored by LCMS. After completion solvent was concentrated and dried under reduced pressure to afford 6-(((2R,4S,5R,6R)-4-acetoxy-5-(2-acetoxyacetamido)-6-((1R,2R)-1,2-diacetoxy-3-(2-(4-chlorophenyl)acetamido)propyl)-2-(methoxycarbonyl)tetrahydro-2H-pyran-2-yl)oxy)hexanoic acid (4) as a light yellow sticky syrup. The crude residue obtained was directly used for the next step without further purification. Yield: 0.40 g, Crude; LCMS (ESI) m/z 773.00 [M+H]+.

### Synthesis of (1R,2R)-1-((2R,3R,4S,6R)-4-acetoxy-3-(2-acetoxyacetamido)-6-((6-((2-ethoxy-2-oxoethyl)(prop-2-yn-1-yl)amino)-6-oxohexyl)oxy)-6-(methoxycarbonyl)tetrahydro-2H-pyran-2-yl)-3-(2-(4-chlorophenyl)acetamido)propane-1,2-diyl diacetate (6)

To a solution of 6-(((2R,4S,5R,6R)-4-acetoxy-5-(2-acetoxyacetamido)-6-((1R,2R)-1,2-diacetoxy-3-(2-(4-chlorophenyl)acetamido)propyl)-2-(methoxycarbonyl)tetrahydro-2H-pyran-2-yl)oxy)hexanoic acid (4, 0.40 g, 0.51 mmol) and ethyl prop-2-yn-1-ylglycinate (5, 0.11 g, 0.77 mmol) in N,N dimethylformamide (5 mL), was added 1H-1,2,3-benzotriazol-1-yloxy-tri-1-pyrrolidinylphosphonium hexafluoridophosphate(1-) (0.53 g, 1.03 mmol) and ethylbis(propan-2-yl)amine (0.27 mL, 1.55 mmol) sequentially at 0° C. and reaction mixture was stirred at room temperature for 6 h. Progress of the reaction was monitored by LCMS. After completion, reaction mixture was concentrated under reduced pressure to obtain crude residue which was purified by flash column chromatography using 0-10% methanol in dichloromethane as eluent to afford (1R,2R)-1-((2R,3R,4S,6R)-4-acetoxy-3-(2-acetoxyacetamido)-6-((6-((2-ethoxy-2-oxoethyl)(prop-2-yn-1-yl)amino)-6-oxohexyl)oxy)-6-(methoxycarbonyl)tetrahydro-2H-pyran-2-yl)-3-(2-(4-chlorophenyl)acetamido)propane-1,2-diyl diacetate (6) as a colorless liquid. Yield: 0.18 g, 36%; LCMS m/z 896.05 [M+H]+.

### Synthesis of (2R,4S,5R,6R)-2-((6-((carboxymethyl)(prop-2-yn-1-yl)amino)-6-oxohexyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Compound 100)

To a stirred solution of (1R,2R)-1-((2R,3R,4S,6R)-4-acetoxy-3-(2-acetoxyacetamido)-6-((6-((2-ethoxy-2-oxoethyl)(prop-2-yn-1-yl)amino)-6-oxohexyl)oxy)-6-(methoxycarbonyl)tetrahydro-2H-pyran-2-yl)-3-(2-(4-chlorophenyl)acetamido)propane-1,2-diyl diacetate (6, 0.16 g, 0.18 mmol) in methanol (5.0 mL), was added solution of lithium hydroxide monohydrate (0.026 g, 1.07 mmol) in water (0.5 mL). The reaction mixture was stirred at room temperature for 6 h. The progress of the reaction was monitored by LCMS. After completion the reaction mixture was treated with acidic (Dowex 50, H⁺) to pH˜6 and the suspension was filtered through sintered funnel with methanol washing. The filtrate was concentrated under reduced pressure to get crude. The crude material was purified by prep HPLC using in water/Acetonitrile solvent mixture and 0.1% TFA as additive to afford (2R,4S,5R,6R)-2-((6-((carboxymethyl)(prop-2-yn-1-yl)amino)-6-oxohexyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Compound 100) as an off white solid. Yield: 0.038 g, 31%; LCMS m/z 686.35 [M+H]+; ¹H NMR (400 MHz, Methanol-d₄) δ 7.90 (d, J=7.6 Hz, 1H), 7.32-7.27 (m, 4H), 4.30-4.26 (m, 3H), 4.16 (s, 1H), 4.01 (s, 2H), 3.90-3.68 (m, 5H), 3.63 (dd, J=14.4 & 2.8 Hz, 1H), 3.53 (s, 2H), 3.44-3.35 (m, 3H), 3.27-3.22 (m, 1H), 2.81 (t, J=2.4 Hz, 1H), 2.73-2.70 (m, 1H), 2.66 (t, J=2.4 Hz, 1H), 2.51 (t, J=7.6 Hz, 1H), 2.32 (t, J=7.2 Hz, 1H), 1.75-1.52 (m, 6H), 1.45-1.37 (m, 2H).

### (2R,4S,5R,6R)-6-((1R,2R)-3-(2-(4-chlorophenyl)acetamido)-1,2-dihydroxypropyl)-4-hydroxy-5-(2-hydroxyacetamido)-2-(oct-7-yn-1-yloxy)tetrahydro-2H-pyran-2-carboxylic acid (Compound 110)

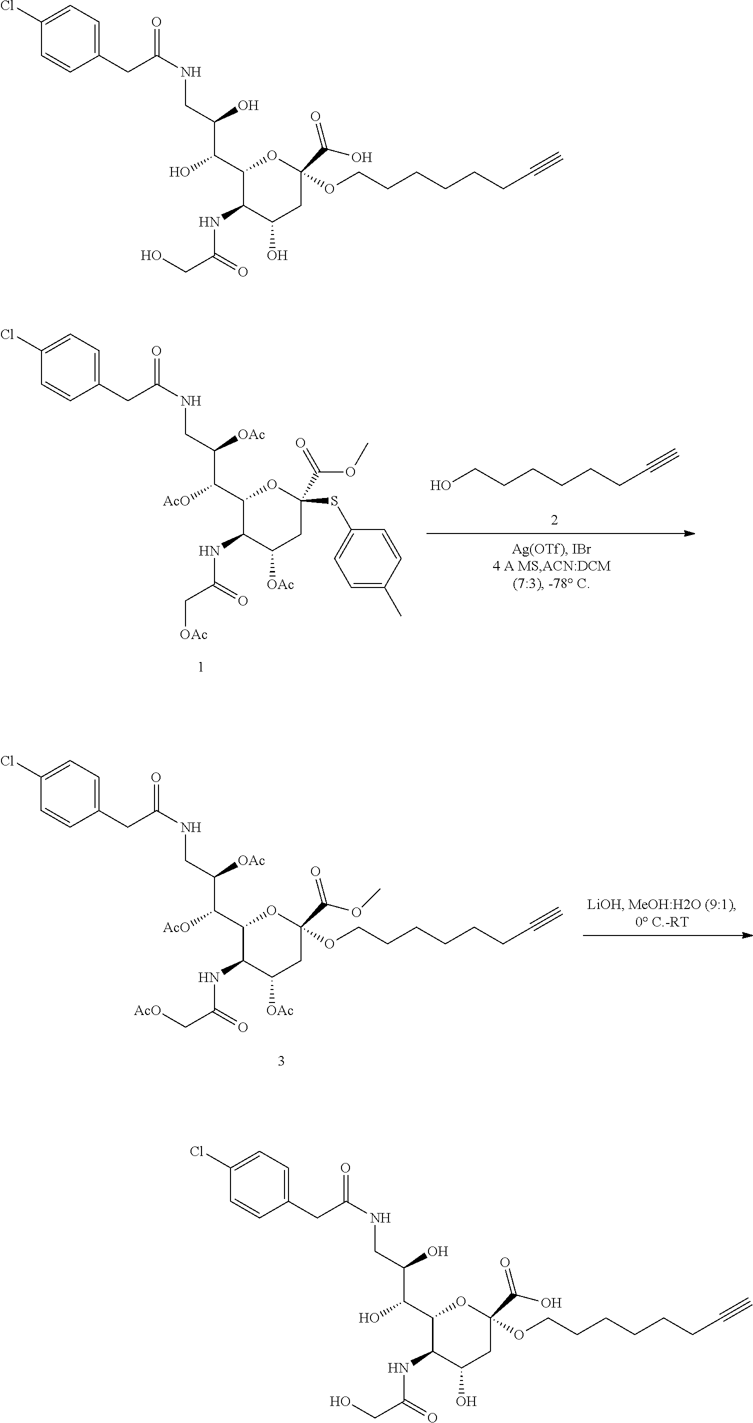

### Synthesis of (1R,2R)-1-((2R,3R,4S,6R)-4-acetoxy-3-(2-acetoxyacetamido)-6-(methoxycarbonyl)-6-(oct-7-yn-1-yloxy)tetrahydro-2H-pyran-2-yl)-3-(2-(4-chlorophenyl)acetamido)propane-1,2-diyl diacetate (3)

A solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (1, 0.50 g, 0.65 mmol) and oct-7-yn-1-ol (2, 0.49 g, 3.92 mmol), silver(1+) trifluoromethanesulfonate (0.50 g, 1.96 mmol) and activated 4 Å powdered molecular sieves (1.00 g) in anhydrous dichloromethane (115 mL) and anhydrous acetonitrile (180 mL) was stirred at room temperature for 1 h under nitrogen atmosphere. The solution was cooled to −78° C. and iodobromane (0.27 g, 1.31 mmol) solution in dichloromethane (5.0 mL) was added dropwise to the reaction mixture and was stirred at same temperature for 2 h. After completion, the reaction mixture was quenched by triethylamine (2.0 mL) and warmed to room temperature. The reaction mixture was filtered through celite and washed with dichloromethane. Filtrate was washed by saturated solution of sodium bicarbonate and dried over sodium sulfate, filtered and concentrated under reduced pressure to get crude compound. The crude product was purified by column chromatography using 80.0 g column and desired compound was eluted with 60-70% ethyl acetate in heptane to afford (1R,2R)-1-((2R,3R,4S,6R)-4-acetoxy-3-(2-acetoxyacetamido)-6-(methoxycarbonyl)-6-(oct-7-yn-1-yloxy)tetrahydro-2H-pyran-2-yl)-3-(2-(4-chlorophenyl)acetamido)propane-1,2-diyl diacetate (3) as a white color solid. Yield: 0.2 g, 26%; LCMS m/z 767.10 [M+H]+.

### Synthesis of (2R,4S,5R,6R)-6-((1R,2R)-3-(2-(4-chlorophenyl)acetamido)-1,2-dihydroxypropyl)-4-hydroxy-5-(2-hydroxyacetamido)-2-(oct-7-yn-1-yloxy)tetrahydro-2H-pyran-2-carboxylic acid (Compound 110)

To a stirred solution of (1R,2R)-1-((2R,3R,4S,6R)-4-acetoxy-3-(2-acetoxyacetamido)-6-(methoxycarbonyl)-6-(oct-7-yn-1-yloxy)tetrahydro-2H-pyran-2-yl)-3-(2-(4-chlorophenyl)acetamido)propane-1,2-diyl diacetate (3, 0.38 g, 0.50 mmol) in methanol (10 mL), was added solution of lithium hydroxide monohydrate (0.074 g, 3.00 mmol) in water (1.0 mL). The reaction mixture was stirred at room temperature for 6 h. The progress of the reaction was monitored by LCMS. After completion the reaction mixture was treated with acidic (Dowex 50, H⁺) to pH ˜6 and the suspension was filtered through celite bed with methanol washing. The filtrate was concentrated under reduced pressure to get crude. The crude material was purified by prep HPLC using 0.1% TFA in water/Acetonitrile to afford (2R,4S,5R,6R)-6-((1R,2R)-3-(2-(4-chlorophenyl)acetamido)-1,2-dihydroxypropyl)-4-hydroxy-5-(2-hydroxyacetamido)-2-(oct-7-yn-1-yloxy)tetrahydro-2H-pyran-2-carboxylic acid (Compound 110) as an off white solid. Yield: 0.063 g, 21%; LCMS m/z 585.35 [M+H]+; ¹H NMR (400 MHz, Methanol-d₄) δ 7.32-7.29 (m, 4H), 4.02 (s, 2H), 3.90-3.68 (m, 5H), 3.64 (dd, J=14.0 Hz & 2.8 Hz, 1H), 3.53 (s, 2H), 3.43-3.35 (m, 2H), 3.26-3.23 (m, 1H), 2.73 (dd, J=12.4 Hz & 4.0 Hz, 1H), 2.17-2.14 (m, 3H), 1.71 (t, J=12.4 Hz, 1H), 1.56-1.45 (m, 4H), 1.43-1.32 (m, 4H).

### (2R,4S,5R,6R)-6-((1R,2R)-3-(2-(4-chlorophenyl)acetamido)-1,2-dihydroxypropyl)-4-hydroxy-2-((6-((hydroxy(prop-2-yn-1-yloxy)phosphoryl)oxy)hexyl)oxy)-5-(2-hydroxyacetamido)tetrahydro-2H-pyran-2-carboxylic acid (Compound 105)

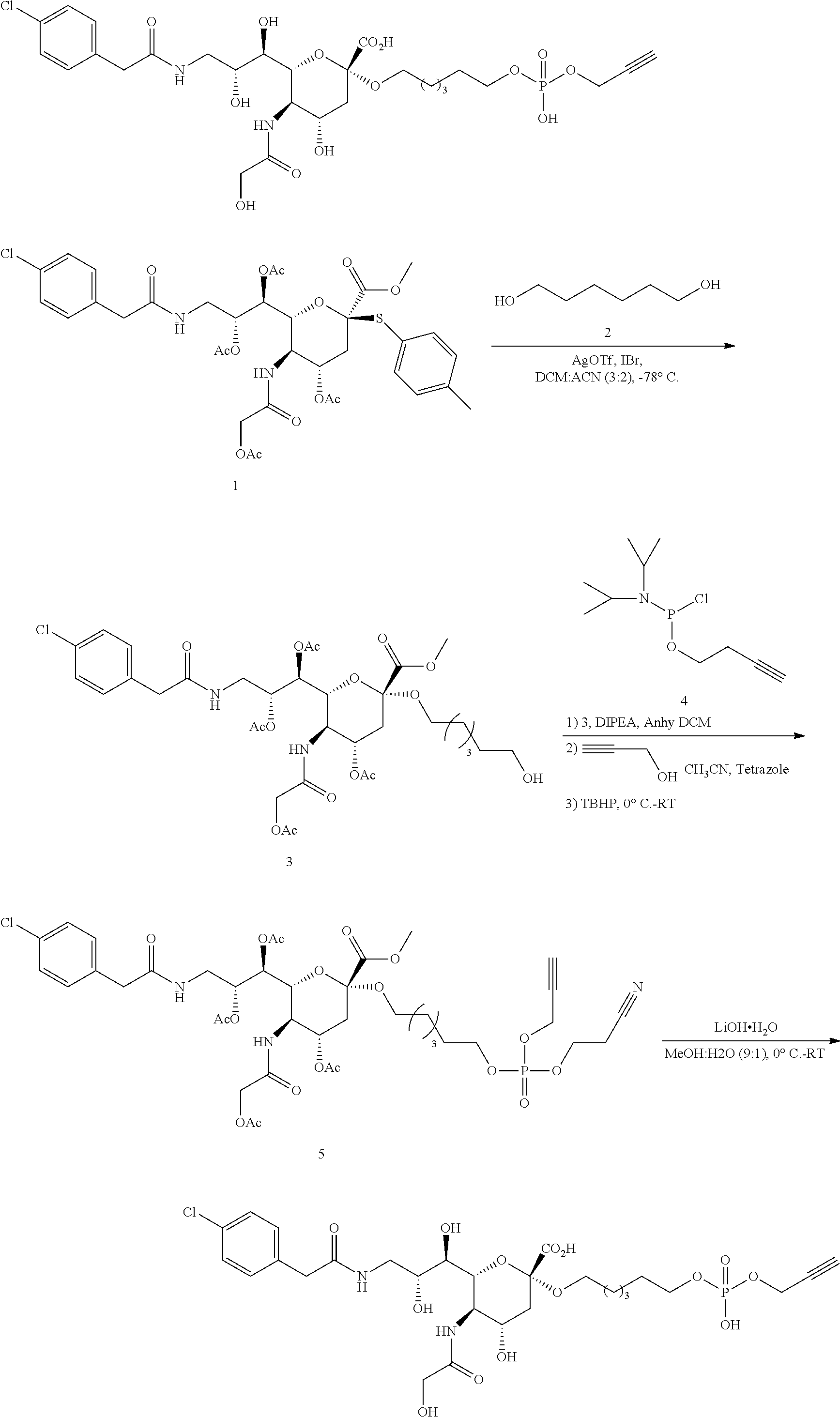

### Synthesis of (1R,2R)-1-((2R,3R,4S,6R)-4-acetoxy-3-(2-acetoxyacetamido)-6-((6-hydroxyhexyl)oxy)-6-(methoxycarbonyl)tetrahydro-2H-pyran-2-yl)-3-(2-(4-chlorophenyl)acetamido)propane-1,2-diyl diacetate (3)

A solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (1, 0.50 g, 0.65 mmol), hexane-1,6-diol (2, 0.54 g, 4.57 mmol), silver trifluoromethanesulfonate (0.50, 1.96 mmol) and activated 4 Å powdered molecular sieves (0.5 g) in anhydrous dichloromethane (75 mL) and acetonitrile (120 mL) was stirred at room temperature for 2 h under nitrogen atmosphere. Iodine mono bromide (0.27 mmol, 1.31 mmol) solution in dichloromethane (5 mL) was added at −78° C. and the reaction mixture was stirred at the same temperature for 2 h. Progress of the reaction was monitored by TLC (100% EA) and LCMS. After completion, the reaction mixture was quenched by triethyl amine (0.1 mL) and warmed to room temperature. The reaction mixture was filtered through sintered funnel washed by dichloromethane. Filtrate was washed by saturated solution of sodium thiosulphate and dried over sodium sulfate, filtered and concentrated under reduced pressure to get crude compound. The crude product was purified by column chromatography using 12.0 g column and desired compound was eluting at 80-100% ethyl acetate in hexane to afford (1R,2R)-1-((2R,3R,4S,6R)-4-acetoxy-3-(2-acetoxyacetamido)-6-((6-hydroxyhexyl)oxy)-6-(methoxycarbonyl)tetrahydro-2H-pyran-2-yl)-3-(2-(4-chlorophenyl)acetamido)propane-1,2-diyl diacetate (3) as a cream colored fluffy solid. Yield: 0.2 g, 21%; LCMS (ESI) m/z 759.05 [M+H]+.

### Synthesis of (1R,2R)-1-((2R,3R,4S,6R)-4-acetoxy-3-(2-acetoxyacetamido)-6-((6-(((2-cyanoethoxy)(prop-2-yn-1-yloxy)phosphoryl)oxy)hexyl)oxy)-6-(methoxycarbonyl)tetrahydro-2H-pyran-2-yl)-3-(2-(4-chlorophenyl)acetamido)propane-1,2-diyl diacetate (5)

To the stirred solution of 3-((chloro(diisopropylamino)phosphaneyl)oxy)propanenitrile (4, 0.09 g, 0.39 mmol) in dichloromethane (4 ml), ethylbis(propan-2-yl)amine (0.11 mL, 0.59 mmol) was added dropwise at 0° C. and the reaction mixture was stirred at same temperature for minutes, then (1R,2R)-1-((2R,3R,4S,6R)-4-acetoxy-3-(2-acetoxyacetamido)-6-((6-hydroxyhexyl)oxy)-6-(methoxycarbonyl)tetrahydro-2H-pyran-2-yl)-3-(2-(4-chlorophenyl)acetamido)propane-1,2-diyl diacetate (3, 0.15 g, 0.19 mmol) in dichloromethane (1 mL) was added to the reaction mixture and the reaction mixture was stirred for another 30 minutes at room temperature. The progress of the reaction was monitored by TLC. After completion, propargyl alcohol (0.022 mL, 0.39 mmol) and tetrazole (0.028 g, 0.39 mmol) in acetonitrile (0.5 mL) were added sequentially at 0° C. and the reaction mixture was stirred for another 1 hr at room temperature. The progress of the reaction was monitored by LCMS. After completion, TBHP (0.07 mL) was added at 0° C. and the reaction mixture was stirred at room temperature for 1 hr. Progress of the reaction mixture was monitored by LCMS. After completion, reaction mixture was directly concentrated under reduced pressure to afford crude (1R,2R)-1-((2R,3R,4S,6R)-4-acetoxy-3-(2-acetoxyacetamido)-6-((6-(((2-cyanoethoxy)(prop-2-yn-1-yloxy)phosphoryl)oxy)hexyl)oxy)-6-(methoxycarbonyl)tetrahydro-2H-pyran-2-yl)-3-(2-(4-chlorophenyl)acetamido)propane-1,2-diyl diacetate (5) as a deep brown viscous syrup which was directly used for the next step without further purification. Yield: 0.15 g, crude; LCMS m/z 914.05 [M+H]+.

### Synthesis of (2R,4S,5R,6R)-6-((1R,2R)-3-(2-(4-chlorophenyl)acetamido)-1,2-dihydroxypropyl)-4-hydroxy-2-((6-((hydroxy(prop-2-yn-1-yloxy)phosphoryl)oxy)hexyl)oxy)-5-(2-hydroxyacetamido)tetrahydro-2H-pyran-2-carboxylic acid (Compound 105)

sTo the solution of (1R,2R)-1-((2R,3R,4S,6R)-4-acetoxy-3-(2-acetoxyacetamido)-6-((6-(((2-cyanoethoxy)(prop-2-yn-1-yloxy)phosphoryl)oxy)hexyl)oxy)-6-(methoxycarbonyl)tetrahydro-2H-pyran-2-yl)-3-(2-(4-chlorophenyl)acetamido)propane-1,2-diyl diacetate (5, 0.15 g, 0.17 mmol) in methanol (5.0 mL) and water (0.5 mL), lithium hydroxide monohydrate (0.023 g, 0.99 mmol) was added at 0° C. The reaction mixture was stirred at room temperature for 3 h. Progress of the reaction was monitored by LCMS. After completion, the reaction mixture was quenched with Dowex-H⁺ resin and filtered with methanol (2-3 times). The organic layer was concentrated under reduced pressure to afford crude residue. The crude was purified by HPLC using 0-100% acetonitrile/water as eluent and the desired fractions were lyophilized to afford (2R,4S,5R,6R)-6-((1R,2R)-3-(2-(4-chlorophenyl)acetamido)-1,2-dihydroxypropyl)-4-hydroxy-2-((6-((hydroxy(prop-2-yn-1-yloxy)phosphoryl)oxy)hexyl)oxy)-5-(2-hydroxyacetamido)tetrahydro-2H-pyran-2-carboxylic acid (Compound 105) as an off-white solid; Yield: 0.014 g, 10%; LCMS m/z 695.4 [M+H]+; ¹H-NMR (400 MHz, Methanol-d4): δ 7.90 (d, J=7.6 Hz, 1H), 7.30 (m, 4H), 4.51 (d, J=6.8 Hz, 2H), 4.01 (s, 2H), 3.94-3.86 (m, 3H), 3.83-3.73 (m, 3H), 3.71-3.68 (m, 1H), 3.63 (dd, J=13.6 Hz & 2.4 Hz, 1H), 3.53 (s, 2H), 3.42-3.40 (m, 1H), 3.38-3.35 (m, 1H), 3.25-3.22 (m, 1H), 2.89 (m, 1H), 2.72 (dd, J=10.8 Hz & 4.0 Hz, 1H), 1.75-1.69 (m, 1H), 1.65-1.63 (m, 2H), 1.56-1.55 (m, 2H), 1.41-1.40 (m, 4H).

### (2R,4S,5R,6R)-6-((1R,2R)-3-(2-(4-chlorophenyl)acetamido)-1,2-dihydroxypropyl)-4-hydroxy-5-(2-oxo-2-(phenylamino)acetamido)-2-((6-(prop-2-yn-1-yloxy)hexyl)oxy)tetrahydro-2H-pyran-2-carboxylic acid (Compound 188)

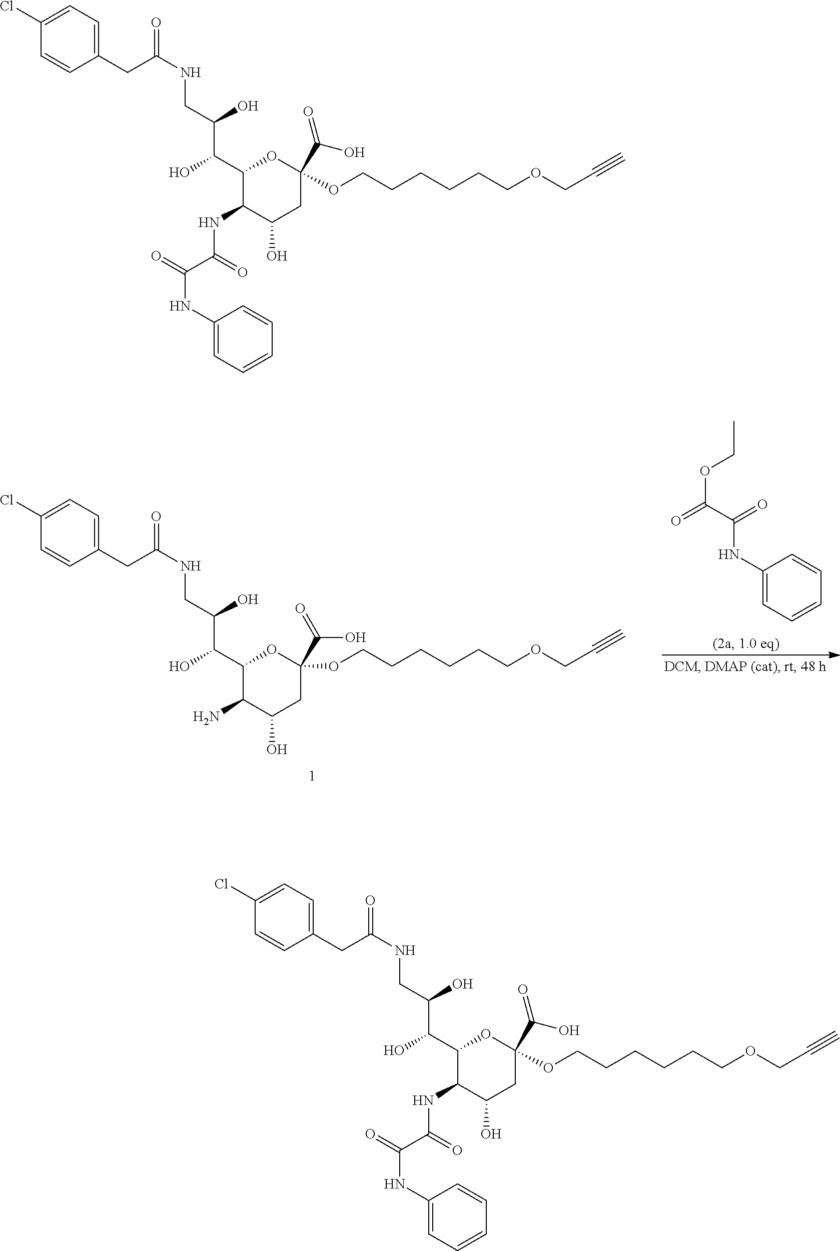 [SMILES: C#CCOCCCCCCO[C@]1(C(=O)O)C[C@H](O)[C@@H](NC(=O)C(=O)Nc2ccccc2)[C@H]([C@H](O)[C@H](O)CNC(=O)Cc2ccc(Cl)cc2)O1 ; C#CCOCCCCCCO[C@]1(C(=O)O)C[C@H](O)[C@@H](N)[C@H]([C@H](O)[C@H](O)CNC(=O)Cc2ccc(Cl)cc2)O1 ; CCOC(=O)C(=O)Nc1ccccc1 ; C#CCOCCCCCCO[C@]1(C(=O)O)C[C@H](O)[C@@H](NC(=O)C(=O)Nc2ccccc2)[C@H]([C@H](O)[C@H](O)CNC(=O)Cc2ccc(Cl)cc2)O1]

### Synthesis of (2R,4S,5R,6R)-6-((1R,2R)-3-(2-(4-chlorophenyl)acetamido)-1,2-dihydroxypropyl)-4-hydroxy-5-(2-oxo-2-(phenylamino)acetamido)-2-((6-(prop-2-yn-1-yloxy)hexyl)oxy)tetrahydro-2H-pyran-2-carboxylic acid (Compound 188)

To a stirred solution of (2R,4S,5R,6R)-5-amino-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (1, 0.1 g, 0.180 mmol) in dry dichloromethane (DCM, 1.0 mL) were sequentially added ethyl 2-oxo-2-(phenylamino)acetate (0.034 g, 0.180 mmol) and 4-Dimethylaminopyridine (DMAP, 0.022 g, 0.180 mmol) and the reaction mixture was stirred for 48 hr at room temperature. Progress of the reaction was monitored by LCMS. After completion, reaction mixture was directly concentrated under reduced pressure to obtain crude residue which was purified by Prep-HPLC using 0-100% acetonitrile/water and the desired fractions were lyophilized to afford (2R,4S,5R,6R)-6-((1R,2R)-3-(2-(4-chlorophenyl)acetamido)-1,2-dihydroxypropyl)-4-hydroxy-5-(2-oxo-2-(phenylamino)acetamido)-2-((6-(prop-2-yn-1-yloxy)hexyl)oxy)tetrahydro-2H-pyran-2-carboxylic acid (Compound 188) as a white solid. Yield: 0.045 g, 35%; LCMS m/z 704.50 [M+H]+; ¹H-NMR (400 MHz, Methanol-d4): δ 7.72-7.70 (m, 2H), 7.35 (t, J=8.4 Hz, 2H), 7.26-7.22 (m, 4H), 7.18-7.14 (m, 1H), 4.13 (d, J=2.4 Hz, 2H), 3.92-3.85 (m, 4H), 3.80-3.75 (m, 1H), 3.61 (dd, J=13.8 Hz & 3.2 Hz, 1H), 3.53-351 (m, 4H), 3.44-3.38 (m, 2H), 3.28-3.27 (m, 1H), 2.81 (t, J=2.4 Hz, 1H), 2.73 (dd, J=12.8 Hz & 3.6 Hz, 1H), 1.78-1.73 (m, 1H), 1.58-1.56 (m, 4H), 1.41-1.36 (m, 4H).

### (2R,4S,5R,6R)-6-((1R,2R)-3-(2-(4-chlorophenyl)acetamido)-1,2-dihydroxypropyl)-4-hydroxy-5-(2-hydroxyacetamido)-2-((6-(1-(15-oxo-15-(perfluorophenoxy)-3,6,9,12-tetraoxapentadecyl)-1H-1,2,3-triazol-4-yl)hexyl)oxy)tetrahydro-2H-pyran-2-carboxylic acid (Compound 110P)

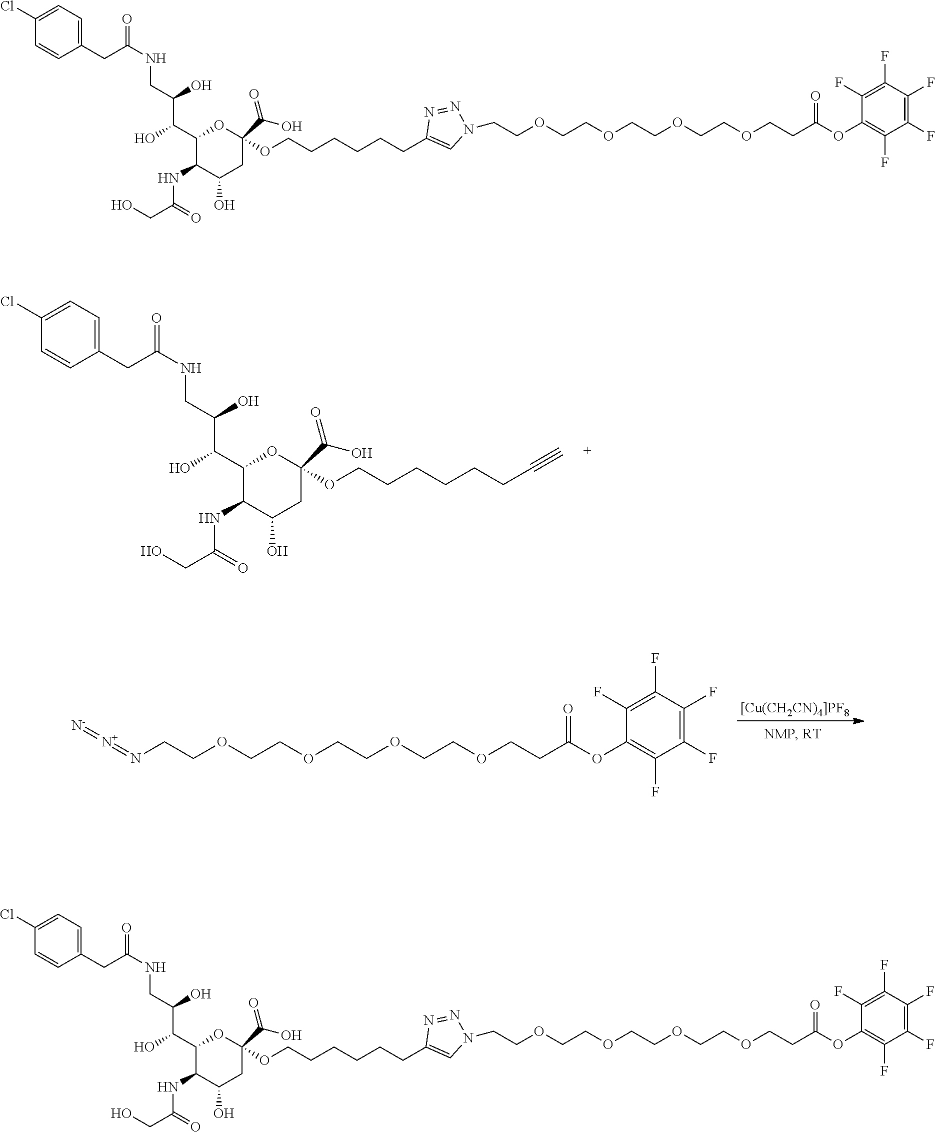

### Synthesis of (2R,4S,5R,6R)-6-((1R,2R)-3-(2-(4-chlorophenyl)acetamido)-1,2-dihydroxypropyl)-4-hydroxy-5-(2-hydroxyacetamido)-2-((6-(1-(15-oxo-15-(perfluorophenoxy)-3,6,9,12-tetraoxapentadecyl)-1H-1,2,3-triazol-4-yl)hexyl)oxy)tetrahydro-2H-pyran-2-carboxylic acid (110P)

To a solution of (2R,4S,5R,6R)-6-((1R,2R)-3-(2-(4-chlorophenyl)acetamido)-1,2-dihydroxypropyl)-4-hydroxy-5-(2-hydroxyacetamido)-2-(oct-7-yn-1-yloxy)tetrahydro-2H-pyran-2-carboxylic acid (110, 70.0 mg, 120 umol) in anhydrous NMP (771 uL) was added Azido-PEG4-PFP ester (65.7 mg, 144 umol) in anhydrous NMP (771 uL) dropwise, followed by Tetrakis(acetonitrile)copper(l) hexafluorophosphate (66.9 mg, 179 umol). The vial was sealed, and the mixture was stirred for 45 min at room temperature. The mixture was filtered through a 0.2 μm PTFE filter, a few drops of acetic acid were added, and the filtrate was purified by C18 reverse phase column chromatography using acetonitrile and water (+0.1% TFA) as eluents. The fractions containing the desired product were lyophilized to afford 110P as a white solid. Yield: 80.4 mg, 64%; LCMS (ESI) m/z 1042 [M+H]+. ¹H NMR (500 MHz, DMSO-d₆): 7.98 (t, J=5.5 Hz, 1H), 7.84 (d, J=7.5 Hz, 1H), 7.79 (s, 1H), 7.31 (d, J=8.5 Hz, 2H), 7.26 (d, J=8.5 Hz, 2H), 4.44 (t, J=5.0 Hz, 2H), 3.86 (m, 2H), 3.79-3.74 (m, 6H), 3.68-3.59 (m, 4H), 3.53-3.51 (m, 6H), 3.51-3.47 (m, 6H), 3.31-3.26 (m, 2H), 3.20 (m, 2H), 3.01 (t, J=6.0 Hz, 2H), 2.97-2.91 (m, 1H), 2.58 (t, J=7.5 Hz, 2H), 1.57-1.54 (m, 2H), 1.54-1.49 (m, 1H), 1.46-1.44 (m, 2H), 1.29-1.27 (m, 4H).

### (2R,4S,5R,6R)-6-((1R,2R)-3-(2-(4-chlorophenyl)acetamido)-1,2-dihydroxypropyl)-2-((6-(1-(18-(2,5-dioxo-2,5-dihydro-1H-pyrrol-1-yl)-15-oxo-3,6,9,12-tetraoxa-16-azaoctadecyl)-1H-1,2,3-triazol-4-yl)hexyl)oxy)-4-hydroxy-5-(2-hydroxyacetamido)tetrahydro-2H-pyran-2-carboxylic acid (Compound 110M)

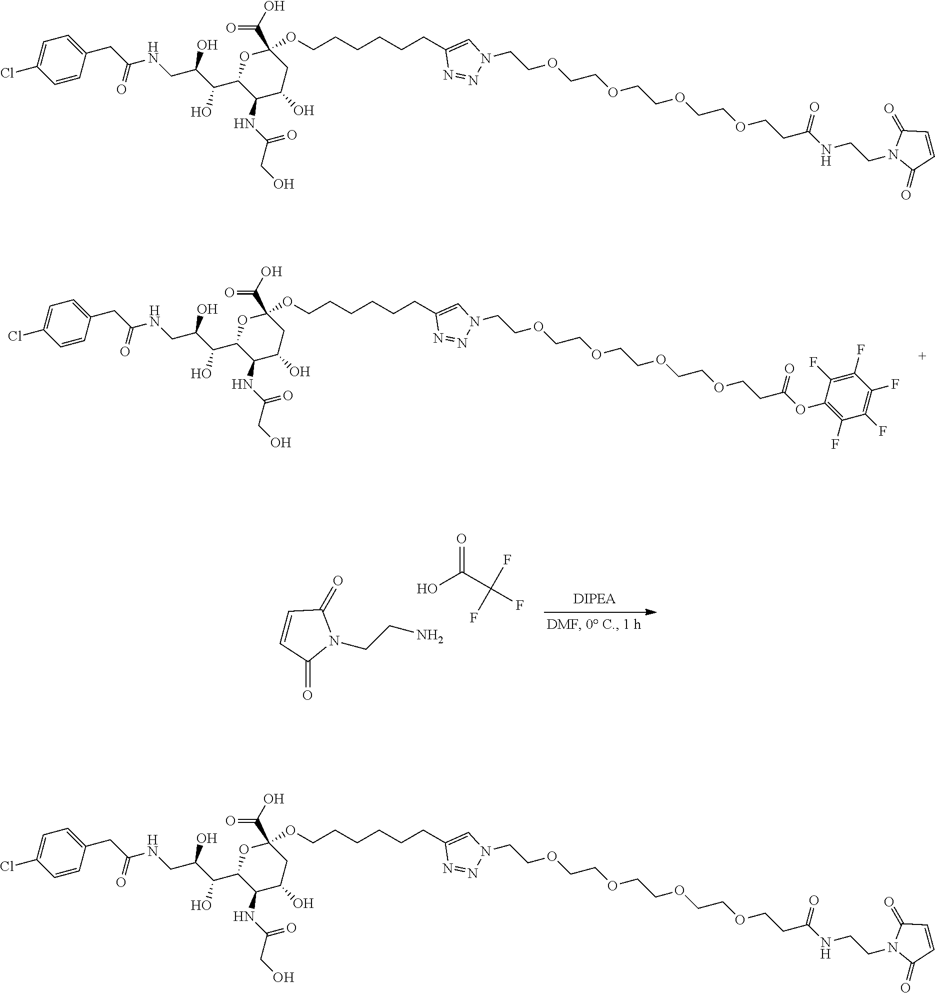 [SMILES: O=C(CCOCCOCCOCCOCCn1cc(CCCCCCO[C@]2(C(=O)O)C[C@H](O)[C@@H](NC(=O)CO)[C@H]([C@H](O)[C@H](O)CNC(=O)Cc3ccc(Cl)cc3)O2)nn1)NCCN1C(=O)C=CC1=O ; O=C(Cc1ccc(Cl)cc1)NC[C@@H](O)[C@@H](O)[C@@H]1O[C@@](OCCCCCCc2cn(CCOCCOCCOCCOCCC(=O)Oc3c(F)c(F)c(F)c(F)c3F)nn2)(C(=O)O)C[C@H](O)[C@H]1NC(=O)CO ; NCCN1C(=O)C=CC1=O ; O=C(O)C(F)(F)F ; O=C(CCOCCOCCOCCOCCn1cc(CCCCCCO[C@]2(C(=O)O)C[C@H](O)[C@@H](NC(=O)CO)[C@H]([C@H](O)[C@H](O)CNC(=O)Cc3ccc(Cl)cc3)O2)nn1)NCCN1C(=O)C=CC1=O]

### Synthesis of (2R,4S,5R,6R)-6-((1R,2R)-3-(2-(4-chlorophenyl)acetamido)-1,2-dihydroxypropyl)-2-((6-(1-(18-(2,5-dioxo-2,5-dihydro-1H-pyrrol-1-yl)-15-oxo-3,6,9,12-tetraoxa-16-azaoctadecyl)-1H-1,2,3-triazol-4-yl)hexyl)oxy)-4-hydroxy-5-(2-hydroxyacetamido)tetrahydro-2H-pyran-2-carboxylic acid (110M)

To a solution of (2R,4S,5R,6R)-6-((1R,2R)-3-(2-(4-chlorophenyl)acetamido)-1,2-dihydroxypropyl)-4-hydroxy-5-(2-hydroxyacetamido)-2-((6-(1-(15-oxo-15-(perfluorophenoxy)-3,6,9,12-tetraoxapentadecyl)-1H-1,2,3-triazol-4-yl)hexyl)oxy)tetrahydro-2H-pyran-2-carboxylic acid (110P, 46.7 mg, 44.8 umol) in DMF (1.08 mL) was added n-(2-aminoethyl)maleimide trifluoroacetate salt (12.5 mg, 49.3 umol). The mixture was cooled down to 0° C. and stirred for 10 min. DIEA (23.4 uL, 134 umol) was added and the mixture was stirred for 1 h at 0° C. The reaction mixture was filtered and purified by preparatory HPLC using acetonitrile and water (+0.05% TFA) as eluents. The fractions containing the desired product were combined and lyophilized to dryness to afford 110M as a white solid. Yield: 35.2 mg, 79%; LCMS (ESI) m/z 998 [M+H]⁺. ¹H NMR (500 MHz, DMSO-d₆): 7.98 (t, J=5.5 Hz, 1H), 7.93 (t, J=6.0 Hz, 1H), 7.85 (d, J=7.5 Hz, 1H), 7.79 (s, 1H), 7.32 (d, J=8.5 Hz, 2H), 7.26 (d, J=8.5 Hz, 2H), 6.99 (s, 2H), 4.45 (t, J=5.0 Hz, 2H), 3.87 (m, 2H), 3.83-3.79 (m, 2H), 3.78 (t, J=5.5 Hz, 2H), 3.68-3.59 (m, 4H), 3.56-3.52 (m, 6H), 3.51-3.47 (m, 10H), 3.31-3.27 (m, 2H), 3.21-3.16 (m, 4H), 2.97-2.92 (m, 1H), 2.58 (t, J=7.5 Hz, 2H), 2.21 (t, J=6.5 Hz, 2H), 1.58-1.55 (m, 2H), 1.54-1.49 (m, 1H), 1.45-1.44 (m, 2H), 1.30-1.28 (m, 4H).

### (1R,2R)-3-(2-([1,1′-biphenyl]-4-yl)acetamido)-1-((2R,3R,4S,6R)-4-acetoxy-3-(2-acetoxyacetamido)-6-(methoxycarbonyl)-6-(p-tolylthio)tetrahydro-2H-pyran-2-yl)propane-1,2-diyl diacetate (Compound 1A)

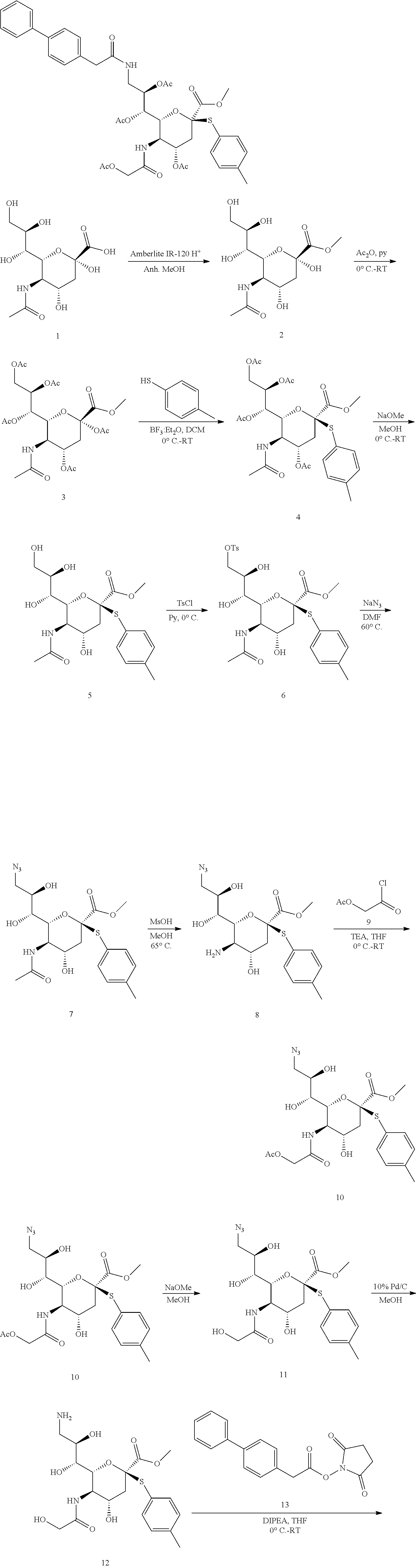

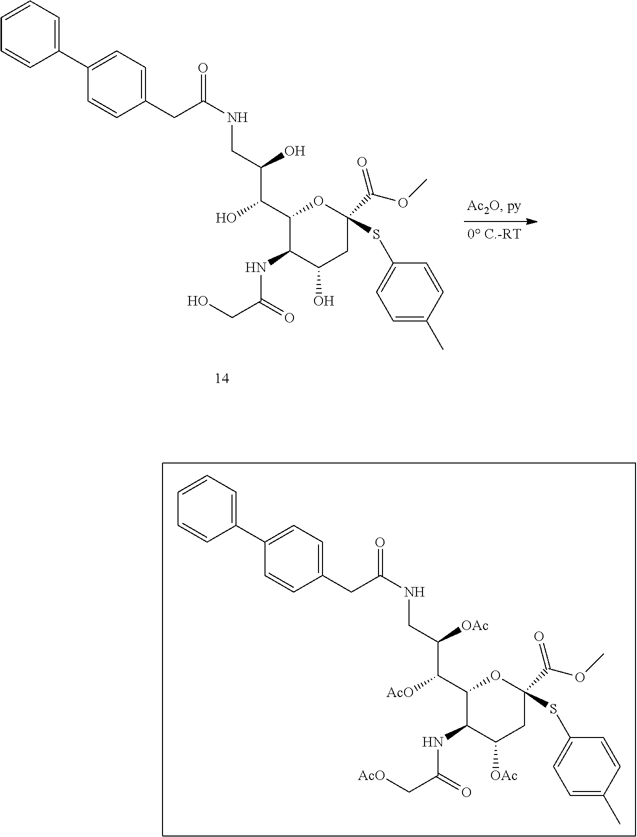 [SMILES: COC(=O)[C@]1(Sc2ccc(C)cc2)C[C@H](O)[C@@H](NC(=O)CO)[C@H]([C@H](O)[C@H](O)CNC(=O)Cc2ccc(-c3ccccc3)cc2)O1 ; COC(=O)[C@]1(Sc2ccc(C)cc2)C[C@H](OC(C)=O)[C@@H](NC(=O)COC(C)=O)[C@H]([C@H](OC(C)=O)[C@@H](CNC(=O)Cc2ccc(-c3ccccc3)cc2)OC(C)=O)O1]

### Synthesis of methyl (2R,4S,5R,6R)-5-acetamido-2,4-dihydroxy-6-((1R,2R)-1,2,3-trihydroxypropyl)tetrahydro-2H-pyran-2-carboxylate (2)

To a stirred suspension of (2R,4S,5R,6R)-5-acetamido-2,4-dihydroxy-6-((1R,2R)-1,2,3-trihydroxypropyl)tetrahydro-2H-pyran-2-carboxylic acid (1, 100.0 g, 323.3 mmol) in anhydrous methanol (2500 mL) was added Amberlite IR-120 (H⁺) resin (80.0 g) at room temperature under argon atmosphere. The reaction mixture was stirred under inert atmosphere until the suspension became a clear solution. The resin was removed by filtration and the filtrate was concentrated under reduced pressure to obtain a residue. The residue was triturated with diethyl ether and filtered to afford methyl (2R,4S,5R,6R)-5-acetamido-2,4-dihydroxy-6-((1R,2R)-1,2,3-trihydroxypropyl)tetrahydro-2H-pyran-2-carboxylate (2) as a light pink solid. Yield: 104.0 g, 99.49%; LCMS (ESI) m/z 324.2 [M+H]⁺.

### Synthesis of (1S,2R)-1-((2R,3R,4S,6S)-3-acetamido-4,6-diacetoxy-6-(methoxycarbonyl)tetrahydro-2H-pyran-2-yl)propane-1,2,3-triyl triacetate (3)

In a 2000 mL round bottom flask, methyl (2R,4S,5R,6R)-5-acetamido-2,4-dihydroxy-6-((1R,2R)-1,2,3-trihydroxypropyl)tetrahydro-2H-pyran-2-carboxylate (2, 102.0 g, 315 mmol) was dissolved with stirring in pyridine (600 mL) under argon atmosphere. To this solution was added acetic anhydride (298 mL, 3.15 mmol) dropwise at 0° C. over 30 min under stirring. The mixture was stirred overnight from 0° C. to room temperature. After completion, the reaction mixture was concentrated on a rotary evaporator. The obtained thick syrup was then poured into a separatory funnel with ethyl acetate (500 mL) and washed with aqueous 1N HCl solution (200 mL) followed by saturated sodium bicarbonate (200 mL) solution and DM water (2×200 mL). The organic layer was separated, dried over anhydrous sodium sulfate, and concentrated under reduced pressure to obtain a thick syrup. The syrup was triturated with diethyl ether and filtered to afford the desired product (1S,2R)-1-((2R,3R,4S,6S)-3-acetamido-4,6-diacetoxy-6-(methoxycarbonyl)tetrahydro-2H-pyran-2-yl)propane-1,2,3-triyl triacetate (3). Yield: 130.0 g, 71.83%; LCMS (ESI) m/z 534.2 [M+H]⁺.

### Synthesis of (1S,2R)-1-((2R,3R,4S,6R)-3-acetamido-4-acetoxy-6-(methoxycarbonyl)-6-(p-tolylthio)tetrahydro-2H-pyran-2-yl)propane-1,2,3-triyl triacetate (4)

Under argon atmosphere, (1S,2R)-1-((2R,3R,4S,6S)-3-acetamido-4,6-diacetoxy-6-(methoxycarbonyl)tetrahydro-2H-pyran-2-yl)propane-1,2,3-triyl triacetate (3, 130.0 g, 243.68 mmol) was dissolved in anhydrous dichloromethane (1300.0 mL) with stirring. To this solution was added activated powdered 4 Å molecular sieves (40.0 g). The reaction mixture was stirred at room temperature for 30 min and cooled to 0° C. followed by the dropwise addition of boron trifluoride diethyl etherate (111.0 mL, 365.5 mmol) over 30 min. The mixture was stirred at room temperature. After completion, the reaction mixture was quenched with triethylamine up to neutral pH, filtered over celite, and washed with dichloromethane (100 mL). To the filtrate was added aqueous sodium bicarbonate (300 mL) with stirring. After 10 min, the organic layer was separated, washed with water (2×300 mL), dried over anhydrous sodium sulfate, and concentrated on a rotary evaporator to obtain a crude residue. The obtained crude residue was purified via column chromatography (60-90% ethyl acetate in hexanes) to afford the desired product (1S,2R)-1-((2R,3R,4S,6R)-3-acetamido-4-acetoxy-6-(methoxycarbonyl)-6-(p-tolylthio)tetrahydro-2H-pyran-2-yl)propane-1,2,3-triyl triacetate (4). Yield: 125.0 g, 85.83%; LCMS (ESI) m/z 598.32 [M+H]⁺.

### Synthesis of methyl (2R,4S,5R,6R)-5-acetamido-4-hydroxy-2-(p-tolylthio)-6-((1R,2R)-1,2,3-trihydroxypropyl)tetrahydro-2H-pyran-2-carboxylate (5)

To a stirred solution of (1S,2R)-1-((2R,3R,4S,6R)-3-acetamido-4-acetoxy-6-(methoxycarbonyl)-6-(p-tolylthio)tetrahydro-2H-pyran-2-yl)propane-1,2,3-triyl triacetate (4, 100.0 g, 167 mmol) in methanol (800 mL) was slowly added sodium methoxide (25% in MeOH) solution (3.58 mL, 16.7 mmol) at 0° C. The reaction mixture was stirred for 2 h at room temperature. After completion, the reaction mixture was cooled to 0° C. and quenched with DOWEX hydrogen form to maintain pH 6. The mixture was filtered through celite, and concentrated under reduced pressure to obtain solids that were then triturated with diethyl ether and filtered to afford methyl (2R,4S,5R,6R)-5-acetamido-4-hydroxy-2-(p-tolylthio)-6-((1R,2R)-1,2,3-trihydroxypropyl)tetrahydro-2H-pyran-2-carboxylate (5). Yield: 71.0 g, 98.80%; LCMS (ESI) m/z 430.10 [M+H]⁺.

### Synthesis of methyl (2R,4S,5R,6R)-5-acetamido-6-((1R,2R)-1,2-dihydroxy-3-(tosyloxy)propyl)-4-hydroxy-2-(p-tolylthio)tetrahydro-2H-pyran-2-carboxylate (6)

To a stirred solution of methyl (2R,4S,5R,6R)-5-acetamido-4-hydroxy-2-(p-tolylthio)-6-((1R,2R)-1,2,3-trihydroxypropyl)tetrahydro-2H-pyran-2-carboxylate (5, 40.0 g, 93.1 mmol) in pyridine (300 mL) was dropwise added a solution of 4-methylbenzene-1-sulfonyl chloride (30.2 g, 158 mmol) in pyridine (100 mL) at 0° C. The resulting reaction solution was stirred overnight. After completion, the reaction mixture was concentrated to obtain a thick syrup. The thick syrup was purified via flash column chromatography (80-95% ethyl acetate in hexanes) to afford methyl (2R,4S,5R,6R)-5-acetamido-6-((1R,2R)-1,2-dihydroxy-3-(tosyloxy)propyl)-4-hydroxy-2-(p-tolylthio)tetrahydro-2H-pyran-2-carboxylate (6). Yield: 21.2 g, 39.0%. LC-MS (ESI) m/z 584.05 [M+H]⁺.

### Synthesis of methyl (2R,4S,5R,6R)-5-acetamido-6-((1R,2R)-3-azido-1,2-dihydroxypropyl)-4-hydroxy-2-(p-tolylthio)tetrahydro-2H-pyran-2-carboxylate (7)

In an inert atmosphere, methyl (2R,4S,5R,6R)-5-acetamido-6-((1R,2R)-1,2-dihydroxy-3-(tosyloxy)propyl)-4-hydroxy-2-(p-tolylthio)tetrahydro-2H-pyran-2-carboxylate (6, 21.0 g, 35.98 mmol) was dissolved under stirring in anhydrous N,N-dimethylformamide (210.0 mL). To this solution was added sodium azide (7.80 g, 120 mmol) at room temperature. The resulting reaction mixture was stirred at 60° C. for 16 h. After completion, the reaction mixture was directly concentrated on a rotary evaporator to obtain a crude solid. The crude solid was purified via column chromatography (80-95% ethyl acetate in hexanes) to afford methyl (2R,4S,5R,6R)-5-acetamido-6-((1R,2R)-3-azido-1,2-dihydroxypropyl)-4-hydroxy-2-(p-tolylthio)tetrahydro-2H-pyran-2-carboxylate (7). Yield: 11.7 g, 71.55%; LCMS (ESI) m/z 453.19 [M−H]⁻.

### Synthesis of methyl (2R,4S,5R,6R)-5-amino-6-((1R,2R)-3-azido-1,2-dihydroxypropyl)-4-hydroxy-2-(p-tolylthio)tetrahydro-2H-pyran-2-carboxylate (8)

To a stirred solution of methyl (2R,4S,5R,6R)-5-acetamido-6-((1R,2R)-3-azido-1,2-dihydroxypropyl)-4-hydroxy-2-(p-tolylthio)tetrahydro-2H-pyran-2-carboxylate (7, 21.0 g, 46.20 mmol) in methanol (210.0 mL) was added methane sulfonic acid (18.0 mL, 277.2 mmol) dropwise at 0° C. The resulting reaction mixture was stirred at 65° C. for 30 h. After completion, the reaction mixture was cooled to 0° C. and quenched with triethylamine (˜15.0 mL, pH 7). The mixture was concentrated under reduced pressure to afford crude methyl (2R,4S,5R,6R)-5-amino-6-((1R,2R)-3-azido-1,2-dihydroxypropyl)-4-hydroxy-2-(p-tolylthio)tetrahydro-2H-pyran-2-carboxylate (8) as a light brown gel. Yield: 19.0 g, 99.68%; LC-MS (ESI) m/z 413.57 [M+H]⁺.

### Synthesis of methyl (2R,4S,5R,6R)-5-(2-acetoxyacetamido)-6-((1R,2R)-3-azido-1,2-dihydroxypropyl)-4-hydroxy-2-(p-tolylthio)tetrahydro-2H-pyran-2-carboxylate (10)

In an inert atmosphere, crude methyl (2R,4S,5R,6R)-5-amino-6-((1R,2R)-3-azido-1,2-dihydroxypropyl)-4-hydroxy-2-(p-tolylthio)tetrahydro-2H-pyran-2-carboxylate (8, 19.0 g, 46.06 mmol) was dissolved under stirring in dry tetrahydrofuran (200.0 mL) and cooled to 0° C. To this solution was slowly added triethylamine (17.70 mL, 138.2 mmol) followed by 2-chloro-2-oxoethyl acetate (9, 4.95 mL, 46.06 mmol) at 0° C. The reaction was stirred at 0° C. for 3 h. The mixture was concentrated under reduced pressure to obtain a crude residue which was then purified via column chromatography (60-75% ethyl acetate in hexanes) to afford methyl (2R,4S,5R,6R)-5-(2-acetoxyacetamido)-6-((1R,2R)-3-azido-1,2-dihydroxypropyl)-4-hydroxy-2-(p-tolylthio)tetrahydro-2H-pyran-2-carboxylate (10). Yield: 15.2 g, 64.38%; LCMS (ESI) m/z 513.42 [M+H]⁺.

### Synthesis of methyl (2R,4S,5R,6R)-6-((1R,2R)-3-azido-1,2-dihydroxypropyl)-4-hydroxy-5-(2-hydroxyacetamido)-2-(p-tolylthio)tetrahydro-2H-pyran-2-carboxylate (11)

To a stirred solution of methyl (2R,4S,5R,6R)-5-(2-acetoxyacetamido)-6-((1R,2R)-3-azido-1,2-dihydroxypropyl)-4-hydroxy-2-(p-tolylthio)tetrahydro-2H-pyran-2-carboxylate (10, 15.0 g, 29.27 mmol) in methanol (150.0 mL) at 0° C. was slowly added sodium methoxide solution (25% in methanol, 0.061 ml, 2.93 mmol). The reaction mixture was stirred for 1 h at room temperature. The reaction mixture was cooled to 0° C. and quenched with DOWEX hydrogen form to maintain pH 6. The mixture was filtered through celite, and concentrated under reduced pressure to obtain solids that were triturated with diethyl ether and filtered on a centered funnel to afford methyl (2R,4S,5R,6R)-6-((1R,2R)-3-azido-1,2-dihydroxypropyl)-4-hydroxy-5-(2-hydroxyacetamido)-2-(p-tolylthio)tetrahydro-2H-pyran-2-carboxylate (11). Yield: 13.0 g, 94.41%; LCMS (ESI) m/z, 471.15 [M+H]⁺.

### Synthesis of methyl (2R,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (12)

To a stirred solution of methyl (2R,4S,5R,6R)-6-((1R,2R)-3-azido-1,2-dihydroxypropyl)-4-hydroxy-5-(2-hydroxyacetamido)-2-(p-tolylthio)tetrahydro-2H-pyran-2-carboxylate (11, 13.0 g, 27.63 mmol) in methanol (130 mL) was added 10% Pd/C (13.0 g, 100% w/w) at room temperature. The reaction was then hydrogenated using balloon pressure of H₂ gas for 12 h. After completion, the reaction was filtered through celite, and the filtrate was concentrated. The obtained residue was then dried under high vacuum to afford crude methyl (2R,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (12) as a thick syrup. Yield: 12.2 g, 99.41%; LCMS (ESI) m/z 445.16 [M+H]⁺.

### Synthesis of methyl (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(p-tolylthio)tetrahydro-2H-pyran-2-carboxylate (14)

To a stirred solution of methyl (2R,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (12, 12.2 g, 27.45 mmol) and 2,5-dioxocyclopentyl 2-([1,1′-biphenyl]-4-yl)acetate (13, 10.19 g, 32.94 mmol) in tetrahydrofuran (40.0 mL) was added ethylbis(propan-2-yl)amine (22.4 mL, 137.23 mmol) at 0° C. The resulting reaction mixture was stirred at room temperature for 12 h. After completion, the mixture was concentrated under reduced pressure to obtain a crude residue. The crude residue was purified via column chromatography (80-90% ethyl acetate in hexanes) to afford methyl (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(p-tolylthio)tetrahydro-2H-pyran-2-carboxylate (14). Yield: 8.0 g, 45.63%; LCMS (ESI) m/z 639.23 [M+H]⁺.

### Synthesis of (1R,2R)-3-(2-([1,1′-biphenyl]-4-yl)acetamido)-1-((2R,3R,4S,6R)-4-acetoxy-3-(2-acetoxyacetamido)-6-(methoxycarbonyl)-6-(p-tolylthio)tetrahydro-2H-pyran-2-yl)propane-1,2-diyl diacetate (Cpd. 1A)

To a stirred solution of methyl (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(p-tolylthio)tetrahydro-2H-pyran-2-carboxylate (14, 8.0 g, 12.52 mmol) in pyridine (80.0 mL) was dropwise added acetic anhydride (11.61 mL, 125.2 mmol) at 0° C. over 30 min. The reaction mixture was stirred overnight from 0° C. to room temperature. After completion, volatiles were removed under vacuum to obtain a crude thick syrup. The crude thick syrup was then poured into a separatory funnel with ethyl acetate (240.0 mL) and washed with 1N HCl solution followed by saturated sodium sulfate solution. The organic layer was dried over anhydrous sodium sulfate and concentrated under reduced pressure to obtain a crude thick syrup. The crude thick syrup was purified via column chromatography (60-70% ethyl acetate in hexanes) to afford the desired product (1R,2R)-3-(2-([1,1′-biphenyl]-4-yl)acetamido)-1-((2R,3R,4S,6R)-4-acetoxy-3-(2-acetoxyacetamido)-6-(methoxycarbonyl)-6-(p-tolylthio)tetrahydro-2H-pyran-2-yl)propane-1,2-diyl diacetate (Cpd. 1A). Yield: 5.40 g, 53.43%; LCMS (ESI) m/z 807.2 [M+H]⁺. ¹H NMR (400 MHz, methanol-d₄) δ 7.27 (d, J=8.4 Hz, 2H), 7.14 (d, J=8.4 Hz, 2H), 4.03 (t, J=8.4 Hz, 1H), 3.77 (d, J=7.6 Hz, 2H), 3.51-3.47 (m, 2H), 3.20 (t, J=6.4 Hz, 2H), 2.91 (dd, J=9.6, 14.4 Hz, 1H), 2.82 (dd, J=6, 14 Hz, 1H), 2.24-2.20 (m, 3H), 2.07 (d, J=9.6 Hz, 1H), 1.75 (d, J=12.8 Hz, 2H), 1.68-1.62 (m, 2H), 1.60-1.57 (m, 2H), 1.56-1.47 (m, 1H).

### (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 2) and (2S,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 3)

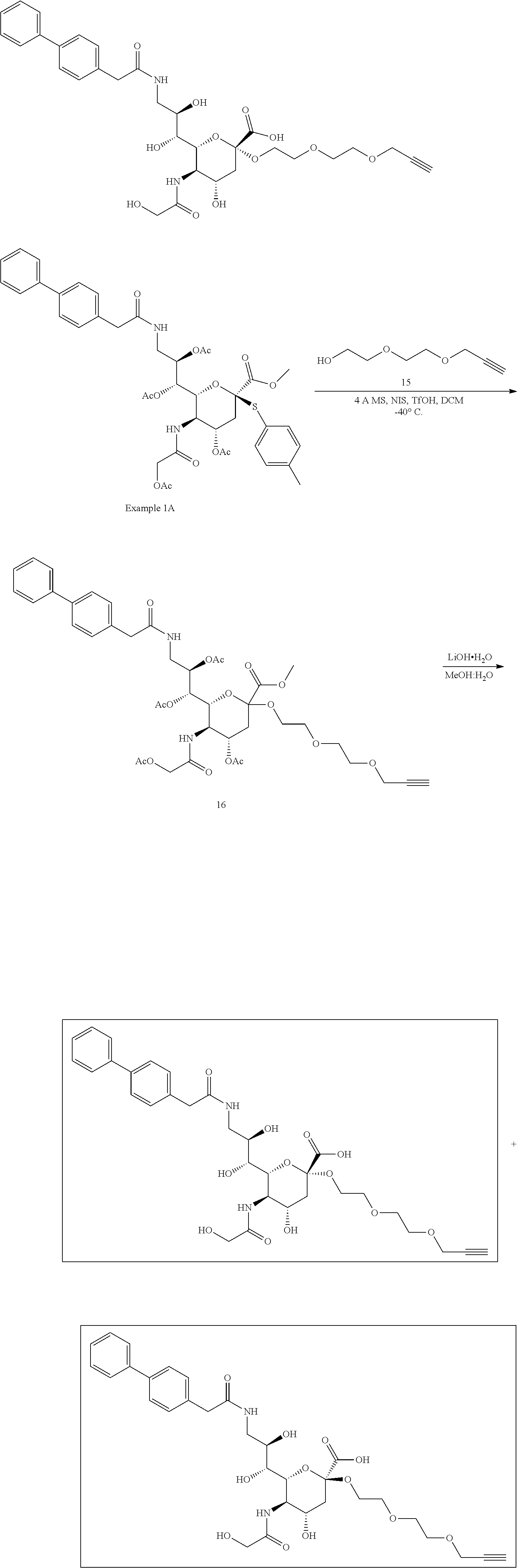

### Synthesis of (1R,2R)-3-(2-([1,1′-biphenyl]-4-yl)acetamido)-1-((2R,3R,4S)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)propane-1,2-diyl diacetate (16)

To a stirred solution of (1R,2R)-3-(2-([1,1′-biphenyl]-4-yl)acetamido)-1-((2R,3R,4S,6S)-4-acetoxy-3-(2-acetoxyacetamido)-6-(methoxycarbonyl)-6-(p-tolylthio)tetrahydro-2H-pyran-2-yl)propane-1,2-diyl diacetate (Cpd. 1, 1.0 g, 1.240 mmol) in anhydrous dichloromethane (20.0 mL) was added 2-(2-(prop-2-yn-1-yloxy)ethoxy)ethan-1-ol (15, 0.893 g, 6.20 mmol) and activated 4 Å powdered molecular sieves (1.0 g, 100% w/w). The resulting reaction solution was stirred at 15 h at room temperature under nitrogen. To the solution was added 1-iodopyrrolidine-2,5-dione (0.697 g, 3.10 mmol) and trifluoromethanesulfonic acid (0.109 mL, 1.240 mmol) at −40° C. The resulting reaction solution was stirred at −40° C. for 1 h. After completion, the reaction mixture was quenched with triethyl amine (0.5 mL) and warmed to room temperature. The reaction mixture was filtered through a sintered funnel and washed with dichloromethane. The filtrate was washed with saturated sodium bicarbonate (aq), dried over sodium sulfate, filtered, and concentrated under reduced pressure to obtain a crude residue. The residue was purified via column chromatography (60-80% ethyl acetate in hexanes) to afford the desired product (1R,2R)-3-(2-([1,1′-biphenyl]-4-yl)acetamido)-1-((2R,3R,4S)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)propane-1,2-diyl diacetate (16) as an anomeric mixture. Yield: 0.80 g, 78.07%; LCMS (ESI) m/z 827.30 [M+H]⁺.

### Synthesis of (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 2) and (2S,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 3)

To a stirred solution of (1R,2R)-3-(2-([1,1′-biphenyl]-4-yl)acetamido)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)propane-1,2-diyl diacetate (3, 0.450 g, 0.544 mmol) in methanol (5.0 mL) was added a solution of Lithium hydroxide monohydrate (0.137 g, 3.27 mmol) in water (0.50 mL). The resulting reaction mixture was stirred at room temperature for 6 h. After completion, the reaction mixture was treated with Dowex 50, H⁺) up to pH ˜6 and the suspension was filtered, and washed with methanol. The filtrate was concentrated under reduced pressure to obtain a crude residue. The residue was purified via preparatory HPLC to afford the desired product (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 2). Yield: 0.316 g, 90%; LCMS (ESI) m/z 645.45 [M+H]⁺. ¹H NMR (400 MHz, Methanol-d₄) δ 7.87 (d, J=7.6 Hz, 1H), 7.61-7.56 (m, 4H), 7.43-7.37 (m, 4H), 7.31 (t, J=7.2 Hz, 1H), 4.17 (d, J=2.4 Hz, 2H), 3.90 (s, 2H), 3.89-3.81 (m, 4H), 3.74-3.59 (m, 11H), 3.39-3.25 (m, 2H), 2.82 (t, J=0.8 Hz, 1H), 2.71 (dd, J=8.8 & 4.0 Hz, 1H), 1.75 (t, J=12.4 Hz, 1H); (2S,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 3). Yield: 0.028 g, 8.0%; LCMS (ESI) m/z 645.42 [M+H]⁺. ¹H NMR (400 MHz, Methanol-d₄) δ 7.82 (d, J=8.8 Hz, 1H), 7.61-7.57 (m, 4H), 7.44-7.36 (m, 4H), 7.31 (t, J=7.2 Hz, 1H), 4.17 (d, J=2.4 Hz, 2H), 4.15-4.119 (m, 1H), 4.03-3.96 (m, 3H), 3.94-3.86 (m, 1H), 3.86-3.78 (m, 2H), 3.36-3.62 (m, 2H) 3.59 (s, 2H), 3.51-3.46 (m, 1H), 3.42-3.35 (m, 2H), 2.84 (t, J=2.4 Hz, 1H), 2.38 (dd, J=12.8 & 3.8 Hz, 1H), 1.66 (t, J=11.6 Hz, 1H).

### (2R,4S,5R,6R)-5-acetamido-6-((1R,2R)-1,2-dihydroxy-3-(3-phenoxybenzamido)propyl)-4-hydroxy-2-(((2R,3R,4S,5R,6R)-3,4,5-trihydroxy-6-(2-(1-(15-oxo-15-(perfluorophenoxy)-3,6,9,12-tetraoxapentadecyl)-1H-1,2,3-triazol-4-yl)ethoxy)tetrahydro-2H-pyran-2-yl)methoxy)tetrahydro-2H-pyran-2-carboxylic acid (Compound 4)

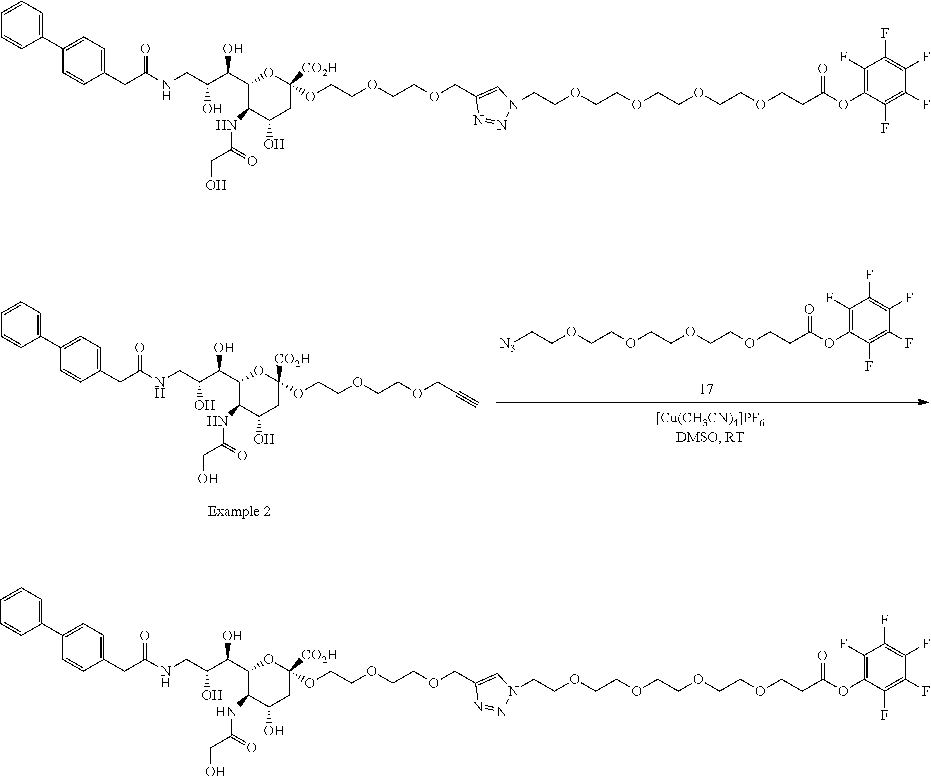

### Synthesis of (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-((1-(15-oxo-15-(perfluorophenoxy)-3,6,9,12-tetraoxapentadecyl)-1H-1,2,3-triazol-4-yl)methoxy)ethoxy)ethoxy)tetrahydro-2H-pyran-2-carboxylic acid (Cpd. 4)

To a stirred solution of (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 2, 0.035 g, 0.054 mmol) and perfluorophenyl 1-azido-3,6,9,12-tetraoxapentadecan-15-oate (17, 0.025 g, 0.054 mmol) in dimethyl sulfoxide (0.5 mL) was added tetrakis(acetonitrile)copper(I) hexafluorophosphate (0.050 g, 0.135 mmol). The resulting reaction mixture was stirred at room temperature for 30 min. After completion, acetic acid (0.3 mL) was added. The resulting solution was diluted with acetonitrile and purified via preparatory HPLC (19-35% acetonitrile in water with 0.1% TFA). Fractions containing the desired product were combined and lyophilized to dryness to afford the desired product (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-((1-(15-oxo-15-(perfluorophenoxy)-3,6,9,12-tetraoxapentadecyl)-1H-1,2,3-triazol-4-yl)methoxy)ethoxy)ethoxy)tetrahydro-2H-pyran-2-carboxylic acid (Cpd. 4) as the TFA salt as a white solid. Yield: 0.022 g, 36.77%; LCMS (ESI) m/z 1102.72 [M+H]⁺; ¹H NMR (400 MHz, DMSO-d₆ with D₂O exchange) δ 7.98 (d, J=3.6 Hz, 2H), 7.59 (d, J=7.2 Hz, 2H), 7.56-7.52 (m, 2H), 7.42 (t, J=8.0 Hz, 2H), 7.33-7.31 (m, 3H), 4.46 (s, 4H), 4.11 (m, 2H), 3.55-3.40 (m, 22H), 3.19 (d, J=9.2 Hz, 2H), 2.99-2.92 (m, 5H), 2.45-2.43 (m, 6H), 2.50 (m, 1H), 1.20 (s, 1H).

### (2R,4S,5R,6R)-6-((1R,2R)-1,2-dihydroxy-3-(2-(4-(pyridin-3-yl)phenyl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 82)

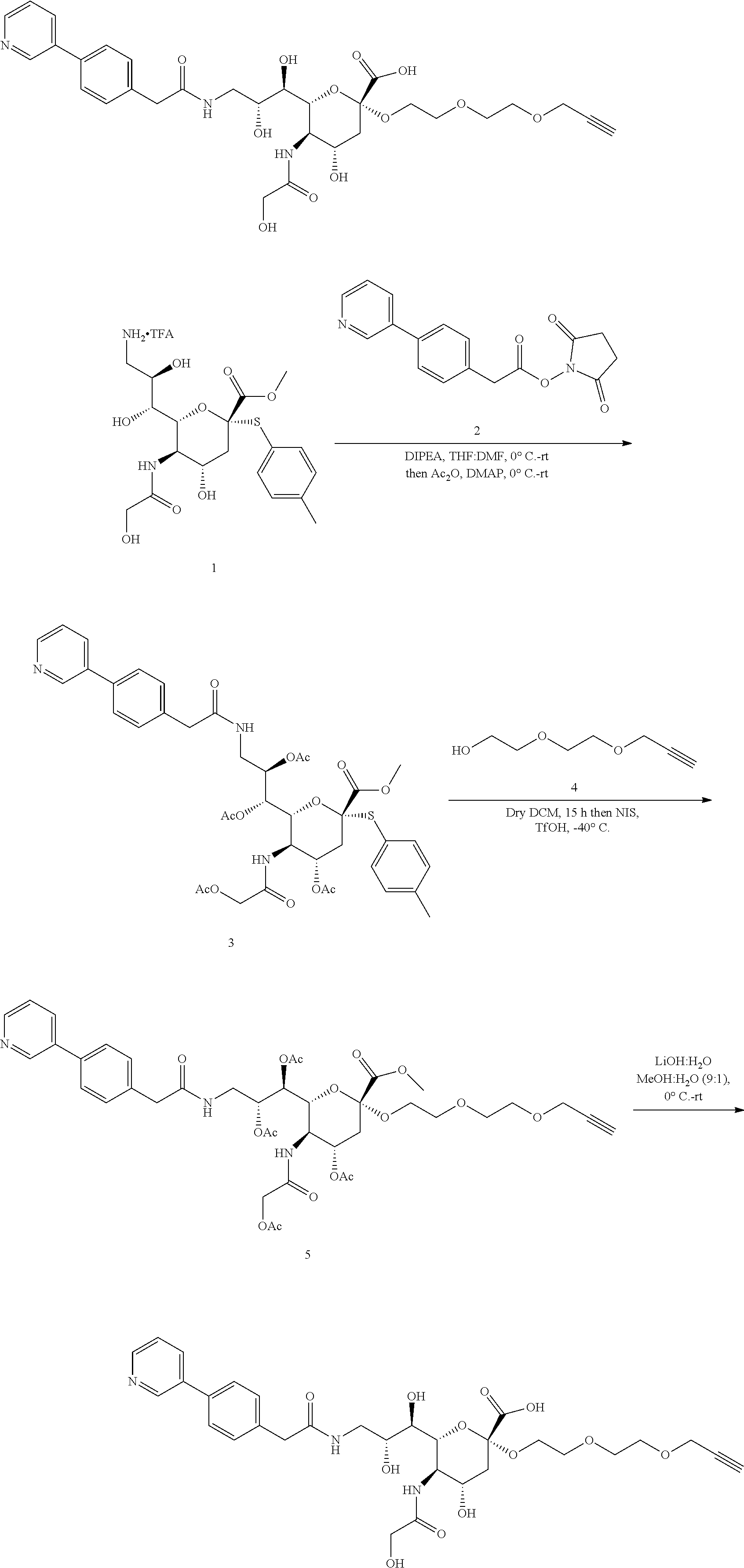

### Synthesis of (1R,2R)-1-((2R,3R,4S,6S)-4-acetoxy-3-(2-acetoxyacetamido)-6-(methoxycarbonyl)-6-(p-tolylthio)tetrahydro-2H-pyran-2-yl)-3-(2-(4-(pyridin-3-yl)phenyl)acetamido)propane-1,2-diyl diacetate (3)

To a stirred solution of methyl (2S,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (1, 1.20 g, 2.70 mmol) and 2,5-dioxopyrrolidin-1-yl 2-(4-(pyridin-3-yl)phenyl)acetate (2, 0.670 g, 2.16 mmol) in tetrahydrofuran (15.00 mL) and N, N-dimethylformamide (2.00 mL) was added ethylbis(propan-2-yl)amine (1.41 mL, 8.10 mmol) at 0° C. then stirred at room temperature for 4 h. Acetic anhydride (2.04 mL, 21.60 mmol) and 4-dimethylaminopyridine (0.032 g, 0.27 mmol) were added at 0° C. and stirred at room temperature for 12 h. The reaction mixture was diluted with water and ethyl acetate. The organic layer was separated, dried, filtered, and concentrated. The crude was purified by silica gel column chromatography using ethyl acetate in hexane as eluents to afford the desired product (3). Yield: 0.80 g, 36.68%; LCMS (ESI) m/z 808.10 [M+H]⁺.

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(4-(pyridin-3-yl)phenyl)acetamido)propane-1,2-diyl diacetate (5)

A mixture of (1R,2R)-1-((2R,3R,4S,6S)-4-acetoxy-3-(2-acetoxyacetamido)-6-(methoxycarbonyl)-6-(p-tolylthio)tetrahydro-2H-pyran-2-yl)-3-(2-(4-(pyridin-3-yl)phenyl)acetamido)propane-1,2-diyl diacetate (3, 0.80 g, 0.99 mmol), 2-(2-(prop-2-yn-1-yloxy)ethoxy)ethan-1-ol (4, 0.356 g, 2.48 mmol) and activated powdered 4 Å molecular sieves (1.00 g, 100% w/w) in anhydrous dichloromethane (15.0 mL) was stirred at room temperature for 15 h. The solution was then cooled to −40° C. followed by addition of 1-iodopyrrolidine-2,5-dione (0.534 g, 2.38 mmol) and trifluoromethanesulfonic acid (0.083 mL, 0.99 mmol). The reaction mixture was stirred at this temperature for another 2 h. After completion, the reaction mixture was quenched by slow addition of triethyl amine (neutral pH) and warmed gradually to room temperature. The reaction mixture was filtered, and the filtrate was further washed with deionized water. The organic layer was separated, dried, filtered, and concentrated. The crude residue obtained was purified by silica gel column chromatography using ethyl acetate in hexane as eluents to afford the desired product (5) as a mixture of anomers. Yield: 0.72 g, 87.83%; LCMS m/z 828.35 [M+H]⁺.

### Synthesis of (2R,4S,5R,6R)-6-((1R,2R)-1,2-dihydroxy-3-(2-(4-(pyridin-3-yl)phenyl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 82)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(p-tolyl)acetamido)propane-1,2-diyl diacetate (5, 0.70 g, 0.92 mmol) in methanol (10.0 mL), was added a solution of lithium hydroxide monohydrate (0.225 g, 5.49 mmol) in water (1.0 mL). The reaction mixture was stirred at room temperature for 4 h. After completion, the reaction mixture was treated with acidic resin (Dowex 50H+) to pH 6 and the suspension was filtered. The filtrate was concentrated, and the crude residue obtained was purified by prep HPLC using acetonitrile in water as eluents (+0.1% trifluoro acetic acid) to afford the desired product (Cpd. 82). Yield: 0.008 g, 1.42%; LCMS m/z 464.40 [M+H]⁺; ¹H NMR (400 MHz, methanol-d₄): δ 8.95 (brs, 1H), 8.62 (d, J=4.4 Hz, 1H), 8.43 (d, J=8.4 Hz, 1H), 7.78-7.75 (m, 1H), 7.69 (d, J=8.4 Hz, 2H), 7.49 (d, J=8.4 Hz, 2H), 4.17 (d, J=2.4 Hz, 2H), 4.00 (s, 2H), 3.91-3.78 (m, 4H), 3.73-3.57 (m, 12H), 3.38 (dd, J=8.8 & 1.2 Hz, 1H), 3.28-3.24 (m, 1H), 2.83 (t, J=2.4 Hz, 1H), 2.73 (dd, J=12.4 & 3.6 Hz, 1H), 1.74 (t, J=12.4 Hz, 1H).

### (2R,4S,5R,6R)-6-((1R,2R)-3-(2-(3′-cyano-[1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 81)

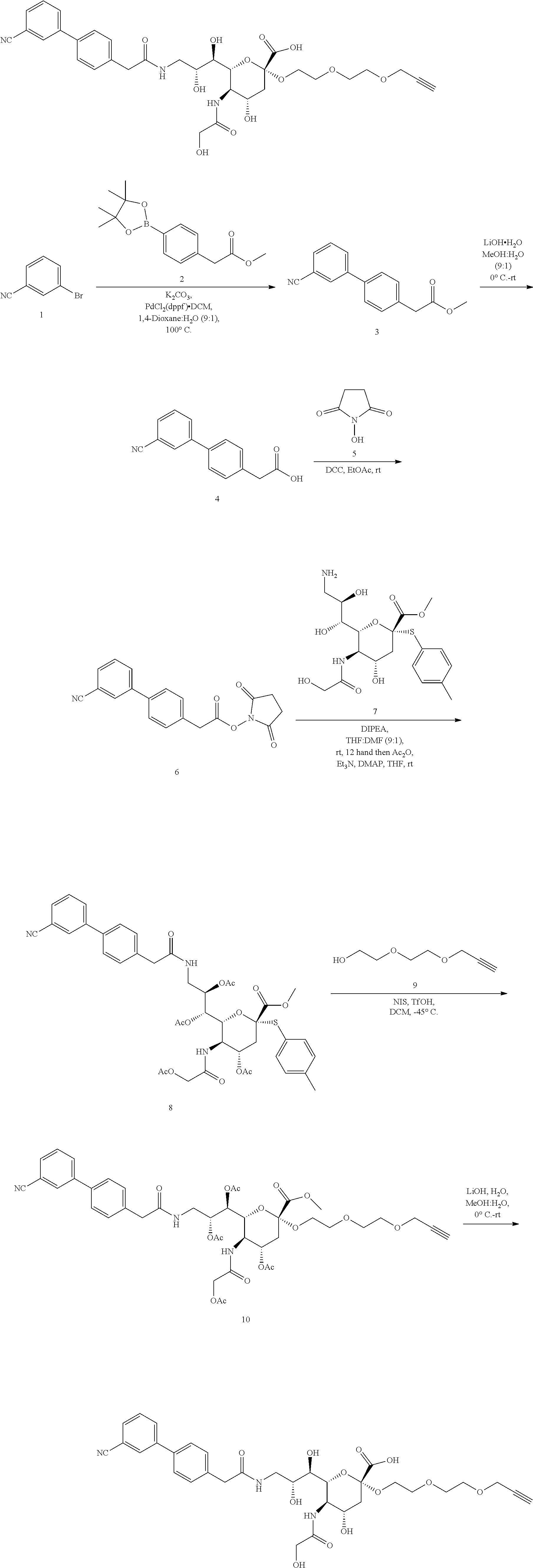

### Synthesis of methyl 2-(3′-cyano-[1,1′-biphenyl]-4-yl)acetate (3)

To a stirred solution of 3-bromobenzonitrile (1, 2.0 g, 11.11 mmol), and methyl 2-(4-(4,4,5,5-tetramnethyl-1,3,2-dioxaborolan-2-yl)phenyl)acetate (2.7 g, 9.78 mmol) in 1,4-dioxane (100.0 mL) and water (10 mL), was added dipotassiun carbonate (4.60 g, 33.33 mmol) was degassed by bubbling argon for 10 mi, [1,1′-Bis(diphenylphosphino)ferrocene]dichloropalladiumn(II) dichloride (0.91 g, 1.11 mmol) was added at room temperature and the mixture was stirred for 5 h at 100° C. After completion, the mixture was diluted with water and ethyl acetate. The organic layer was separated, dried, filtered, and concentrated. The crude residue obtained was purified by silica gel column chromatography using ethyl acetate in hexane as eluents to afford the desired product (3). Yield: 1.60 g, 57.9%; LCMS (ESI) m/z 252.1 [M+H]⁺.

### Synthesis of 2-(3′-cyano-[1,1′-biphenyl]-4-yl)acetic acid (4)

To a stirred solution of methyl 2-(3′-cyano-[1,1′-biphenyl]-4-yl)acetate (2, 1.0 g, 3.98 mmol) in methanol (10.0 mL) and water (1 mL) was added lithium hydroxide monohydrate (0.49 g, 11.94 mmol) at 0° C. and stirred at room temperature for 4 h. After completion of the reaction, the mixture was acidified by addition of 1N HCl solution and diluted with water, ethyl acetate. The organic layer was separated, dried, filtered, and concentrated to afford the desired product (4). Yield: 0.50 g, 52.96%; LCMS (ESI) m/z 238.1 [M+H]⁺.

### Synthesis of 2,5-dioxopyrrolidin-1-yl 2-(3′-cyano-[1,1′-biphenyl]-4-yl)acetate (6)

To a stirred solution of 2-(3′-cyano-[1,1′-biphenyl]-4-yl)acetic acid (4, 1.5 g, 6.33 mmol) in ethyl acetate (50 mL), was added 1-hydroxypyrrolidine-2,5-dione (5, 0.73 g, 6.33 mmol) and N,N′-dicyclohexylcarbodiimide (1.30 g, 6.33 mmol) at 0° C. then stirred at room temperature for 6 h. After completion the mixture was concentrated. The residue obtained was dissolved in ethyl acetate, filtered, and the filtrate was concentrated. The crude was purified by silica gel column chromatography using ethyl acetate in hexane as eluents to afford the desired product (6). Yield: 1.30 g, 61.50%; LCMS (ESI) m/z 335.13 [M+H]⁺.

### Synthesis of (1R,2R)-1-((2R,3R,4S,6S)-4-acetoxy-3-(2-acetoxyacetamido)-6-(methoxycarbonyl)-6-(p-tolylthio)tetrahydro-2H-pyran-2-yl)-3-(2-(3′-cyano-[1,1′-biphenyl]-4-yl)acetamido)propane-1,2-diyl diacetate (8)

To a stirred solution of methyl (2S,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (7, 1.50 g, 3.38 mmol) and 2,5-dioxopyrrolidin-1-yl 2-(3′-cyano-[1,1′-biphenyl]-4-yl)acetate (5, 0.902 g, 2.70 mmol) in tetrahydrofuran (10.00 mL) and N,N-dimethylformamide (2.00 mL), was added ethylbis(propan-2-yl)amine (1.76 mL, 10.14 mmol) at 0° C. then stirred at room temperature for 4 h. Acetic anhydride (3.19 mL, 33.88 mmol) and 4-Dimethylaminopyridine (0.041 g, 0.34 mmol) were added at 0° C. and the resulting mixture was stirred at room temperature for 12 h. After completion the mixture was diluted with water and ethyl acetate. The organic layer was separated, dried, filtered, and concentrated. The crude was purified by silica gel column chromatography using 80-90% ethyl acetate in hexane as eluents to afford the desired product (8). Yield: 1.0 g, 35.62%; LCMS (ESI) m/z 832.05 [M+H]⁺.

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(3′-cyano-[1,1′-biphenyl]-4-yl)acetamido)propane-1,2-diyl diacetate (10)

A mixture of (1R,2R)-1-((2R,3R,4S,6S)-4-acetoxy-3-(2-acetoxyacetamido)-6-(methoxycarbonyl)-6-(p-tolylthio)tetrahydro-2H-pyran-2-yl)-3-(2-(3′-cyano-[1,1′-biphenyl]-4-yl)acetamido)propane-1,2-diyl diacetate (8, 1.0 g, 1.20 mmol), 2-(2-(prop-2-yn-1-yloxy)ethoxy)ethan-1-ol (9, 0.424 g, 3.00 mmol) and activated powdered 4 Å molecular sieves (1.00 g, 100% w/w) in anhydrous dichloromethane (15.0 mL) was stirred at room temperature for 15 h. The solution was then cooled to −45° C. followed by addition of 1-iodopyrrolidine-2,5-dione (0.677 g, 3.00 mmol) and trifluoromethanesulfonic acid (0.16 mL, 1.20 mmol). The reaction mixture was stirred at this temperature for another 2 h. After completion, the reaction mixture was quenched by slow addition of triethyl amine (neutral pH) and warmed gradually to room temperature. The reaction mixture was filtered, and the filtrate was further washed with deionized water. The organic layer was separated, dried, filtered, and concentrated. The crude residue obtained was purified by silica gel column chromatography using ethyl acetate in hexane as eluents to afford the desired product (10). Yield: 0.60 g, 58.59%; LCMS m/z 852.1 [M+H]⁺.

### Synthesis of (2R,4S,5R,6R)-6-((1R,2R)-3-(2-(3′-cyano-[1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 81)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(3′-cyano-[1,1′-biphenyl]-4-yl)acetamido)propane-1,2-diyl diacetate (10, 0.50 g, 0.61 mmol) in methanol (10.0 mL), was added a solution of lithium hydroxide monohydrate (0.14 g, 3.63 mmol) in water (0.5 mL). The reaction mixture was stirred at room temperature for 6 h. After completion, the reaction mixture was treated with acidic resin (Dowex 50H+) to pH˜6 and the suspension was filtered. The filtrate was concentrated, and the crude residue obtained was purified by prep HPLC using acetonitrile in water (+0.1% trifluoroacetic acid) as eluents to afford the desired product (Cpd. 81). Yield: 0.083 g, 21.12%; LCMS m/z 692.45 [M+H3O]+; ¹H NMR (400 MHz, methanol-d₄): δ 7.99 (s, 1H), 7.94 (d, J=8.0 Hz, 1H), 7.69 (d, J=8.0 Hz, 1H), 7.64-7.60 (m, 3H), 7.43 (d, J=8.0 Hz, 1H), 4.17 (d, J=2.4 Hz, 2H), 4.00 (brs, 2H), 3.90-3.78 (m, 4H), 3.74-3.57 (m, 11H), 3.38 (d, J=. 8.0 Hz, 1H), 3.26-3.24 (m, 1H), 2.83 (brs, 1H), 2.72 (dd, J=14.0 & 4.8 Hz, 1H), 1.75 (t, J=12.0 Hz, 12.0 Hz, 1H).

### (2R,4S,5R,6R)-6-((1R,2R)-1,2-dihydroxy-3-(2-(4-(2-oxo-1,2-dihydropyridin-4-yl)phenyl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 75)

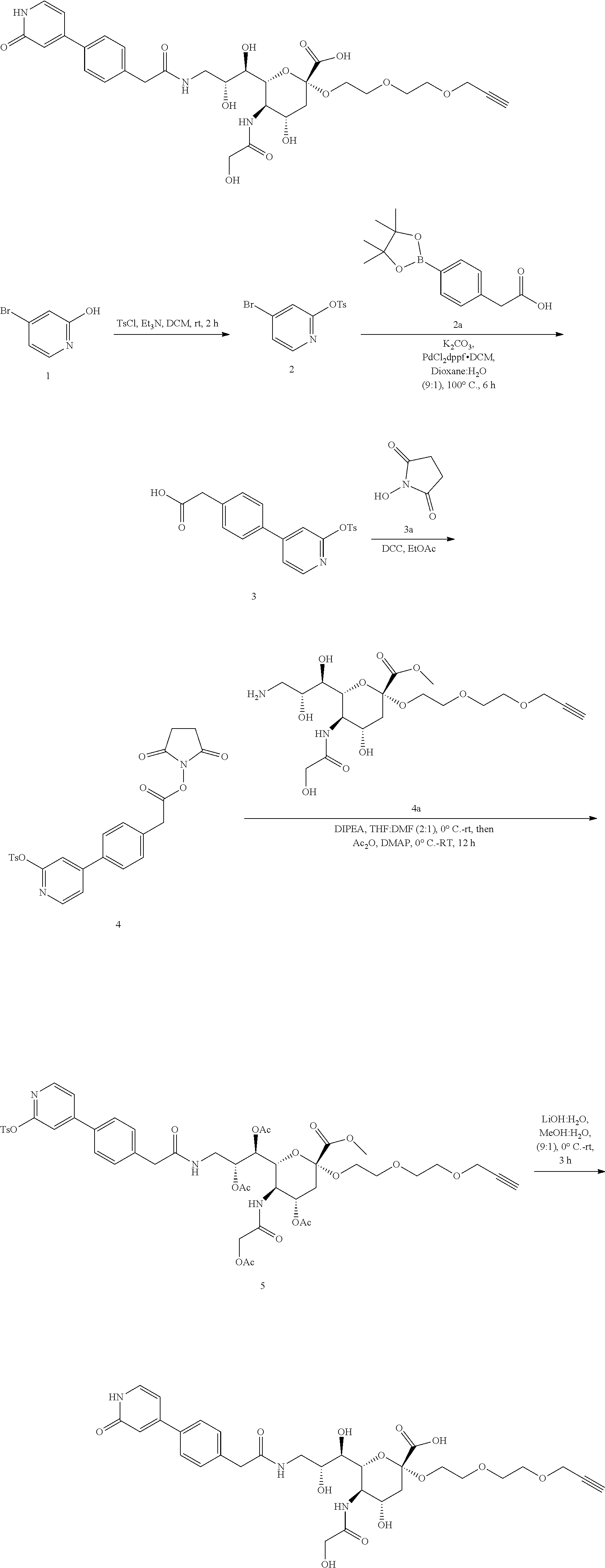

### Synthesis of 4-bromopyridin-2-yl 4-methylbenzenesulfonate (2)

To a stirred solution of 4-bromopyridin-2-ol (1, 2.00 g, 11.50 mmol) in dichloromethane (20.0 mL), were successively added 4-methylbenzene-1-sulfonyl chloride (2.63 g, 13.8 mmol), triethylamine (1.35 mL, 23.0 mmol) and N,N-dimethylpyridin-4-amine (0.140 g, 115 mmol) at 0° C. and the mixture was stirred at room temperature for 2 h. After completion the mixture was diluted with water and ethyl acetate. The organic layer was separated, dried, filtered, and concentrated. The crude was purified by silica gel column chromatography using ethyl acetate in hexane as eluents to afford the desired product (2). Yield: 2.30 g, 60.97%; LCMS (ESI) m/z 329.85 [M+H]⁺.

### Synthesis of 2-(4-(2-(tosyloxy)pyridin-4-yl)phenyl)acetic acid (3)

A mixture of 4-bromopyridin-2-yl 4-methylbenzenesulfonate (2, 1.25 g, 3.820 mmol), 2-(4-(4,4,5,5-tetramethyl-1,3,2-dioxaborolan-2-yl)phenyl)acetic acid (2a, 1.20 g, 4.58 mmol) and potassium carbonate (1.58 g, 14.400 mmol) in 1,4-dioxane (12.0 mL) and water (2.0 mL) was degassed by bubbling argon for 10 min. 1,1-bis(diphenylphosphino)ferrocene dichloropalladium(II) (0.417 g, 0.511 mmol) was added at room temperature and the mixture was stirred for 6 h at 100° C. After completion the mixture was diluted with water and ethyl acetate. The organic layer was separated, dried, filtered, and concentrated. The crude was purified by silica gel column chromatography using ethyl acetate in hexane as eluents to afford the desired product (3). Yield: 1.00 g, 68.47%; LCMS m/z 384.10 [M+H]⁺.

### Synthesis of 2,5-dioxopyrrolidin-1-yl 2-(4-(2-(tosyloxy)pyridin-4-yl)phenyl)acetate (4)

To a stirred solution of 2-(4-(2-(tosyloxy)pyridin-4-yl)phenyl)acetic acid (3, 1.50 g, 3.91 mmol) in ethyl acetate(15.0 mL), were added 1-hydroxypyrrolidine-2,5-dione (0.675 g, 5.870 mmol) and N,N′-dicyclohexylcarbodiimide (1.21 g, 5.870 mmol) at 0° C. and the reaction was stirred at room temperature for 6 h. After completion the mixture was filtered, washed with ethyl acetate, and the filtrate was concentrated. The crude was purified by silica gel column chromatography using ethyl acetate in hexane as eluents to afford the desired product (4). Yield: 1.50 g, 79.80%; LCMS m/z 481.05 [M+H]⁺.

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(4-(2-(tosyloxy)pyridin-4-yl)phenyl)acetamido)propane-1,2-diyl diacetate (5)

To a stirred solution of methyl (2R,4S,5R,6R)-6-((1R,2R)-3-amino-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylate (4a, 0.50 g, 1.080 mmol) and 2,5-dioxopyrrolidin-1-yl 2-(4-(2-(tosyloxy)pyridin-4-yl)phenyl)acetate (4, 0.414 g, 0.861 mmol) in tetrahydrofuran (4.0 mL) and N, N-dimethylformamide (2.00 mL), was added ethylbis(propan-2-yl)amine (0.93 mL, 5.380 mmol) at 0° C. then stirred at room temperature for 4 h. Acetic anhydride (1.02 mL, 10.800 mmol) and N, N-dimethyl aniline (0.065 g, 0.538 mmol) were added at 0° C. and the resulting mixture was stirred at room temperature for 12 h. After completion the mixture was diluted with water and ethyl acetate. The organic layer was separated, dried, filtered, and concentrated. The crude was purified by silica gel column chromatography using ethyl acetate in hexane as eluents to afford the desired product (5). Yield: 0.220 g, 24.63%; LCMS (ESI) m/z 998.30 [M+H]⁺.

### Synthesis of (2R,4S,5R,6R)-6-((1R,2R)-1,2-dihydroxy-3-(2-(4-(2-oxo-1,2-dihydropyridin-4-yl)phenyl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 75)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(4-(2-(tosyloxy)pyridin-4-yl)phenyl)acetamido)propane-1,2-diyl diacetate (5, 0.200 g, 0.200 mmol) in methanol (5.0 mL), was added a solution of lithium hydroxide monohydrate (0.059 g, 1.420 mmol) in water (0.5 mL). The reaction mixture was stirred at room temperature for 6 h. After completion, the reaction mixture was treated with acidic resin (Dowex 50H+) to pH ˜6 and the suspension was filtered. The filtrate was concentrated, and the crude residue obtained was purified by prep HPLC using acetonitrile in water (+0.1% TFA) as eluents to afford the desired product (Cpd. 75). Yield: 0.0035 g, 2.64%; LCMS m/z 662.34 [M+H]⁺; ¹H NMR (400 MHz, methanol-d4): δ 7.64 (d, J=8.4 Hz, 2H), 7.49 (d, J=6.8 Hz, 1H), 7.45 (d, J=8.4 Hz, 2H), 6.76-6.73 (m, 2H), 4.21-4.17 (m, 3H), 4.03 (s, 1H), 4.00 (s, 2H), 3.92-3.89 (m, 2H), 3.80-3.76 (m, 2H), 3.70-3.66 (m, 5H), 3.65-3.59 (m, 10H), 3.31-3.21 (m, 2H), 2.82-2.79 (m, 2H), 1.66 (t, J=6.8 Hz, 1H).

### (2R,4S,5R,6R)-5-(2,2-difluoroacetamido)-6-((1R,2R)-1,2-dihydroxy-3-(2-(4′-hydroxy-[1,1′-biphenyl]-4-yl)acetamido)propyl)-4-hydroxy-2-(2-(2-(prop-2-yn-1-yloxy)ethoxy)ethoxy)tetrahydro-2H-pyran-2-carboxylic acid (Compound 76)

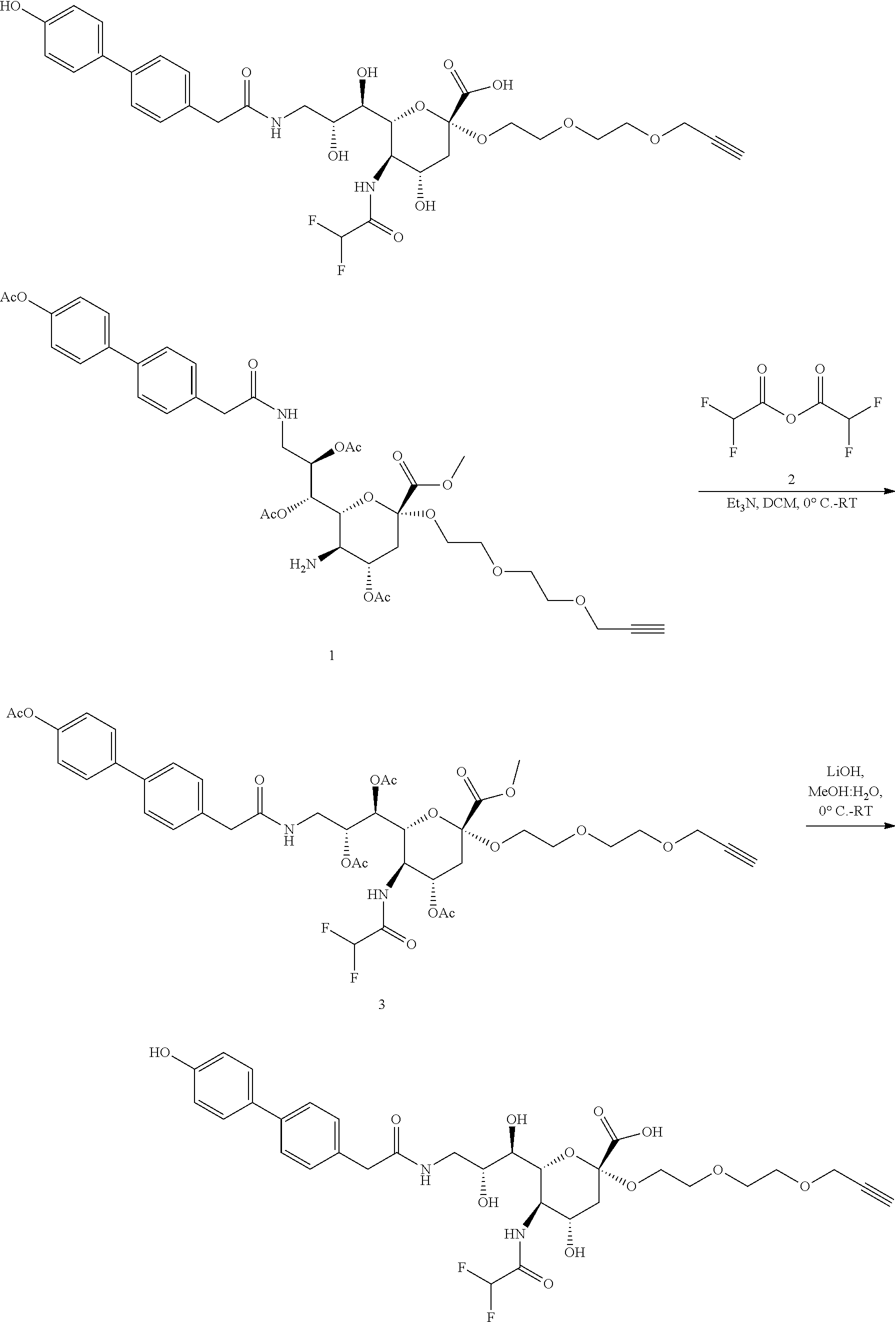

### Synthesis of (1R,2R)-1-((2R,3R,4S,6R)-4-acetoxy-3-(2,2-difluoroacetamido)-6-(methoxycarbonyl)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (3)

To a stirred solution of (1R,2R)-1-((2R,3R,4S,6R)-4-acetoxy-3-amino-6-(methoxycarbonyl)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (1, 0.10 g, 0.13 mmol) in dichloromethane (2.0 mL) was added triethylamine (0.086 mL, 0.64 mmol) followed by 2,2-difluoroacetic anhydride (0.018 g, 0.13 mmol) slowly at 0° C. The reaction mixture was stirred at room temperature for 3 h. After completion, the reaction mixture was concentrated, and the crude residue obtained was purified by silica gel column chromatography using ethyl acetate in hexane as eluents to afford the desired product (3) as a mixture of anomers. Yield: 0.075 g, 68.2%; LCMS (ESI) m/z 860.65 [M−H]⁻.

### Synthesis of (2R,4S,5R,6R)-5-(2,2-difluoroacetamido)-6-((1R,2R)-1,2-dihydroxy-3-(2-(4′-hydroxy-[1,1′-biphenyl]-4-yl)acetamido)propyl)-4-hydroxy-2-(2-(2-(prop-2-yn-1-yloxy)ethoxy)ethoxy)tetrahydro-2H-pyran-2-carboxylic acid (Cpd. 76)

To a stirred solution of (1R,2R)-1-((2R,3R,4S,6R)-4-acetoxy-3-(2,2-difluoroacetamido)-6-(methoxycarbonyl)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (3, 0.075 g, 0.09 mmol) in methanol (2 mL) was added a solution of lithium hydroxide monohydrate (0.021 g, 0.52 mmol) in water (0.1 mL). The reaction mixture was stirred at room temperature for 3 h. After completion, the reaction mixture was treated with acidic resin (Dowex 50, H⁺) to pH ˜6 and the suspension was filtered. The filtrate was concentrated, and the crude residue obtained was purified by prep HPLC using acetonitrile in water (+0.1% TFA) as eluents to afford the desired product (Cpd. 76). Yield: 0.010 g, 17%; LCMS m/z 681.20 [M+H]⁺; ¹H NMR (400 MHz, methanol-d₄): δ 7.49 (d, J=8.4 Hz, 2H), 7.43 (d, J=8.8 Hz, 2H), 7.32 (d, J=8.4 Hz, 2H), 6.84 (d, J=8.8 Hz, 2H), 6.01 (t, J=54.0 Hz, 1H), 4.20-4.13 (m, 2H), 3.91-3.51 (m, 16H), 3.25-3.20 (m, 1H), 2.82 (t, J=2.4 Hz, 1H), 2.75 (dd, J=12.8 Hz, 4.4 Hz, 1H), 1.70 (t, J=12.8 Hz, 1H).

### (2R,4S,5R,6R)-6-((1R,2R)-3-(2-(3-chloro-5-hydroxyphenyl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 129)

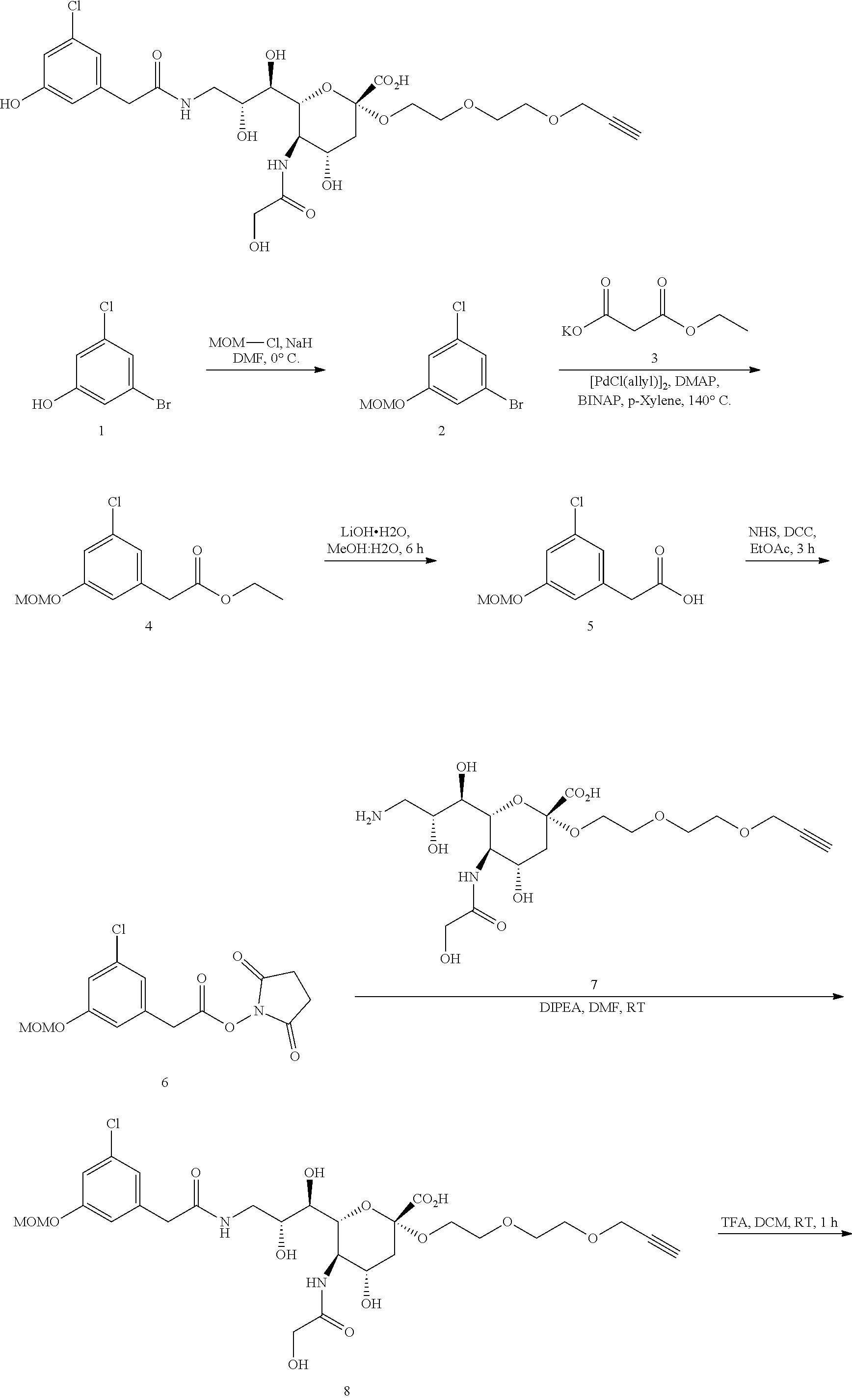

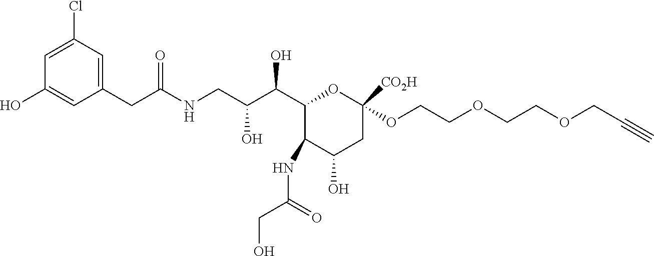 [SMILES: C#CCOCCOCCO[C@]1(C(=O)O)C[C@H](O)[C@@H](NC(=O)CO)[C@H]([C@H](O)[C@H](O)CNC(=O)Cc2cc(O)cc(Cl)c2)O1]

### Synthesis of 1-bromo-3-chloro-5-(methoxymethoxy)benzene (2)

To a stirred solution of 3-bromo-5-chlorophenol (1, 5.0 g, 24.10 mmol) in N,N-dimethylformamide (50.0 mL) was added sodium hydride 60% (2.30 g, 36.20 mmol) at 0° C. and the mixture was stirred for 30 min at this temperature. Chloro(methoxy)methane (2.75 mL, 36.20 mmol) was added, and the resulting mixture was stirred for 3 h at 0° C. After completion, the reaction mixture was quenched with saturated solution of ammonium chloride and extracted with ethyl acetate. The combined organic layers were dried, filtered, and the filtrate was concentrated. The crude residue was purified by silica gel column chromatography using ethyl acetate in heptane as eluents to afford the desired product (2). Yield: 6.0 g, 99%; ¹H NMR (400 MHz, CDCl₃): δ 7.15 (t, J=1.6 Hz, 1H), 7.10 (t, J=2.0 Hz, 1H), 6.99 (t, J=2.0 Hz, 1H), 5.13 (s, 2H), 3.45 (s, 3H).

### Synthesis of ethyl 2-(3-chloro-5-(methoxymethoxy)phenyl)acetate (4)

A mixture of 1-bromo-3-chloro-5-(methoxymethoxy)benzene (2, 2.0 g, 7.95 mmol), potassium 3-ethoxy-3-oxopropanoate (3, 2.71 g, 15.90 mmol), N,N-dimethylpyridin-4-amine (0.097 g, 0.795 mmol) and 2,2′-Bis(diphenylphosphino)-1,1′-binaphthalene (0.248 g, 0.398 mmol) in m-xylene (20.0 mL) was degassed at room temperature by bubbling nitrogen for 15 min. Allyl palladium chloride dimer (0.286 g, 0.795 mmol) was added was stirred at 130° C. for 12 h. After completion, the reaction mixture was filtered through celite, and the filtrate was concentrated. The crude residue was purified by silica gel column chromatography using ethyl acetate in heptane as eluents to afford the desired (2). Yield: 1.0 g, 49%; ¹H NMR (400 MHz, CDCl₃): δ 6.96 (t, J=2.0 Hz, 1H), 6.93 (s, 1H), 6.85 (s, 1H), 5.14 (s, 2H), 4.18-4.13 (m, 2H), 3.58 (s, 2H), 3.47 (s, 3H), 1.25 (t, J=7.2 Hz, 1H).

### Synthesis of 2-(3-chloro-5-(methoxymethoxy)phenyl)acetic acid (5)

To a stirred solution of ethyl 2-(3-chloro-5-(methoxymethoxy)phenyl)acetate (4, 0.50 g, 1.93 mmol) in ethanol (5.0 mL) was added a solution of lithium hydroxide monohydrate (0.092 g, 3.87 mmol) in water (1.0 mL). The reaction mixture was stirred at room temperature for 5 h. After completion, the reaction mixture was concentrated, and the mixture was treated with 1N HCl solution. The product was collected by filtration, washed with water and diethyl ether, then dried under high vacuum to afford the desired product (5). Yield: 0.40 g, 90%; LCMS (ESI) m/z 229.00 [M−H]⁻.

### Synthesis of 2,5-dioxopyrrolidin-1-yl 2-(3-chloro-5-(methoxymethoxy)phenyl)acetate (6)

To a stirred solution of 2-(3-chloro-5-(methoxymethoxy)phenyl)acetic acid (5, 0.40 g, 1.73 mmol) in ethyl acetate (5.0 mL), were added 1-hydroxypyrrolidine-2,5-dione (0.199 g, 1.73 mmol) and N,N′-dicyclohexylmethanediimine (0.356 g, 1.73 mmol) at 0° C. then stirred at room temperature for 4 h. After completion, the reaction mixture was concentrated, and residue was dissolved in ethyl acetate and the product was collected by filtration and washed with ethyl acetate. The solid was dried under high vacuum to afford the desired product (6). Yield: 0.300 g, 53%; LCMS (ESI) m/z 325.85 [M−H]⁻.

### Synthesis of (2R,4S,5R,6R)-6-((1R,2R)-3-(2-(3-chloro-5-(methoxymethoxy)phenyl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (8)

To a stirred solution of (2R,4S,5R,6R)-6-((1R,2R)-3-amino-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (7, 0.20 g, 0.444 mmol) and 2,5-dioxopyrrolidin-1-yl 2-(3-chloro-5-(methoxymethoxy)phenyl)acetate (6, 0.145 g, 0.444 mmol) in N,N-dimethylformamide (3.0 mL) was added ethylbis(propan-2-yl)amine (0.23 mL, 1.33 mmol) at 0° C. then stirred at room temperature for 4 h. After completion, the reaction mixture was concentrated, and the crude was purified by trituration with diethyl ether to afford the desired product (8). Yield: 0.21 g; 71%; LCMS (ESI) m/z 463.10 [M+H]⁺;

### Synthesis of (2R,4S,5R,6R)-6-((1R,2R)-3-(2-(3-chloro-5-hydroxyphenyl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 129)

To a stirred solution of (2R,4S,5R,6R)-6-((1R,2R)-3-(2-(3-chloro-5-(methoxymethoxy)phenyl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (8, 0.20 g, 0.301 mmol) in dichloromethane (2.0 ml), was added trifluoroacetic acid (1.0 mL) at 0° C. and the mixture was stirred at room temperature for 2 h. After completion, the reaction mixture was concentrated. The crude residue obtained was purified by prep HPLC to afford the desired product (Cpd. 129). Yield: 0.050 g, 27%; LCMS (ESI) m/z 617.30 [M−H]⁻. ¹H NMR (400 MHz, Methanol-d₄): δ 6.81 (s, 1H), 6.69-6.68 (m, 2H), 4.21 (d, J=2.4 Hz, 2H), 4.04 (s, 2H), 3.94-3.89 (m, 2H), 3.87-3.80 (m, 2H), 3.76-3.74 (m, 1H), 3.71-3.60 (m, 8H), 3.46 (s, 2H), 3.40-3.36 (m, 1H), 3.26-3.21 (m, 1H), 2.85 (t, J=2.4 Hz, 1H), 2.76-2.73 (m, 1H), 1.77 (t, J=12.0 Hz, 1H).

### (2R,4S,5R,6R)-2-((4-(but-3-yn-1-yloxy)benzyl)oxy)-6-((1R,2R)-1,2-dihydroxy-3-(4-(4-(trifluoromethyl)phenyl)tetrahydro-2H-pyran-4-carboxamido)propyl)-4-hydroxy-5-(2-hydroxyacetamido)tetrahydro-2H-pyran-2-carboxylic acid (Compound 170)

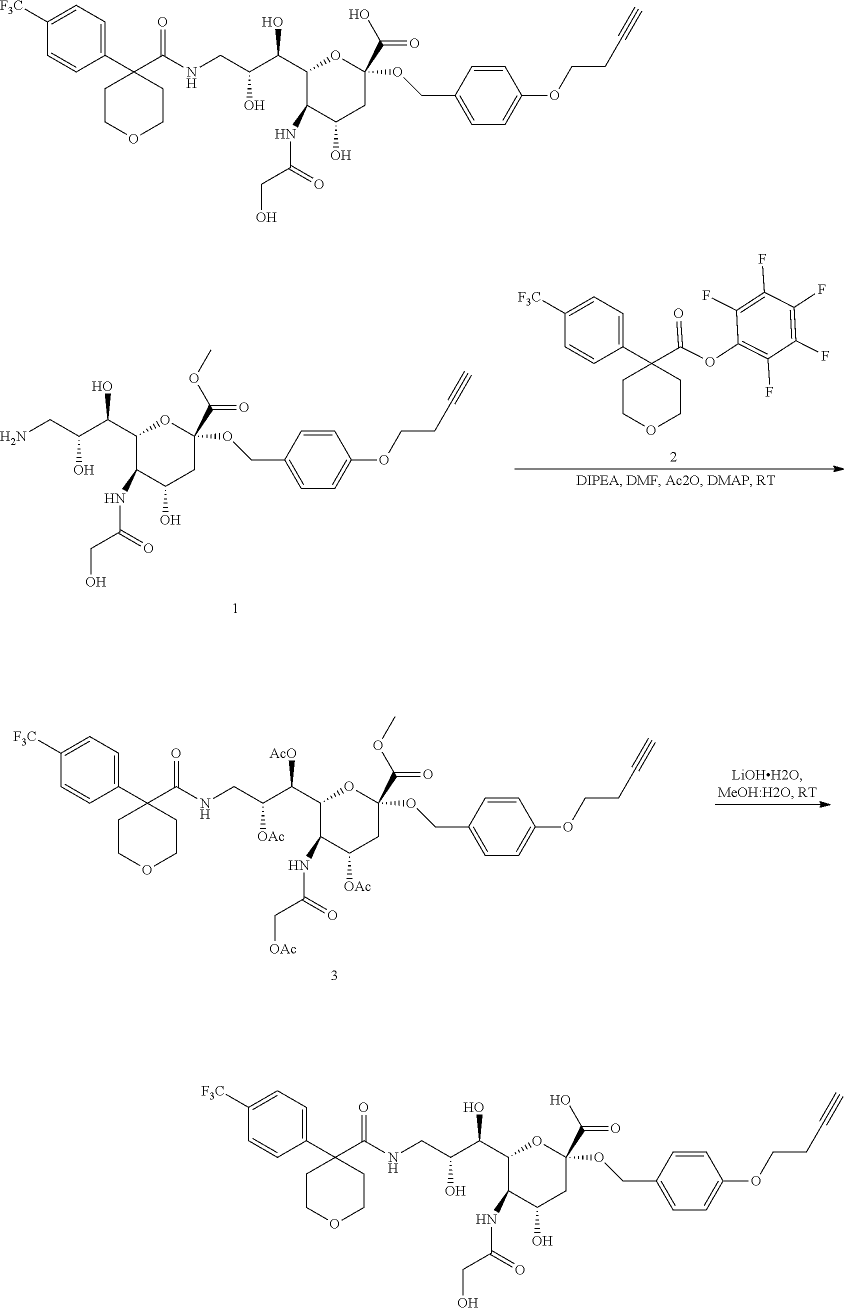

### Synthesis of (1R,2R)-1-((2R,3R,4S,6R)-4-acetoxy-3-(2-acetoxyacetamido)-6-((4-(but-3-yn-1-yloxy)benzyl)oxy)-6-(methoxycarbonyl)tetrahydro-2H-pyran-2-yl)-3-(4-(4-(trifluoromethyl)phenyl)tetrahydro-2H-pyran-4-carboxamido)propane-1,2-diyl diacetate (3)

To a stirred solution of methyl (2R,4S,5R,6R)-6-((1R,2R)-3-amino-1,2-dihydroxypropyl)-2-((4-(but-3-yn-1-yloxy)benzyl)oxy)-4-hydroxy-5-(2-hydroxyacetamido)tetrahydro-2H-pyran-2-carboxylate (1, 0.30 g, 0.604 mmol) and perfluorophenyl 4-(4-(trifluoromethyl)phenyl)tetrahydro-2H-pyran-4-carboxylate (2, 0.266 g, 0.604 mmol) in N, N-dimethylformamide (3.0 mL) was added ethylbis(propan-2-yl)amine (0.52 mL, 3.02 mmol) at 0° C. then stirred at room temperature for 4 h. After completion acetic anhydride (0.68 mL, 6.04 mmol) and 4-(dimethylamino) pyridine (0.007 g, 0.060 mmol) were added and allowed to stir for 12 h at room temperature then concentrated. The crude residue was diluted with ethyl acetate and washed with saturated solution of sodium bicarbonate. The organic layer was dried, filtered, and the filtrate was concentrated. The crude residue was purified by silica gel column chromatography using ethyl acetate in heptane as eluents to afford the desired product (3). Yield: 0.20 g, 36%; LCMS m/z 921.05 [M+H]⁺.

### Synthesis of (2R,4S,5R,6R)-2-((4-(but-3-yn-1-yloxy)benzyl)oxy)-6-((1R,2R)-1,2-dihydroxy-3-(4-(4-(trifluoromethyl)phenyl)tetrahydro-2H-pyran-4-carboxamido)propyl)-4-hydroxy-5-(2-hydroxyacetamido)tetrahydro-2H-pyran-2-carboxylic acid (Cpd. 170)

To a stirred solution of (1R,2R)-1-((2R,3R,4S,6R)-4-acetoxy-3-(2-acetoxyacetamido)-6-((4-(but-3-yn-1-yloxy)benzyl)oxy)-6-(methoxycarbonyl)tetrahydro-2H-pyran-2-yl)-3-(4-(4-(trifluoromethyl)phenyl)tetrahydro-2H-pyran-4-carboxamido)propane-1,2-diyl diacetate (0.20 g, 0.217 mmol) in methanol (3.0 ml) was added a solution of lithium hydroxide monohydrate (0.054 g, 1.30 mmol) in water (0.50 ml). The reaction mixture was stirred at room temperature for 6 h. After completion, the reaction mixture was treated with acidic resin (Dowex 50, H⁺) to pH˜6 and the suspension was filtered and washed with methanol. The filtrate was concentrated, and the crude residue obtained was purified by prep HPLC to afford the desired product (Cpd. 170). Yield: 0.007 g, 4.36%; LCMS (ESI) m/z 739.60 [M+H]⁺. ¹H NMR (400 MHz, Methanol-d₄): δ 7.60 (s, 4H), 7.24 (d, J=8.8 Hz, 2H), 6.90-6.86 (m, 2H), 4.70 (d, J=10.8 Hz, 1H), 4.38 (d, J=10.8 Hz, 1H), 4.07 (t, J=6.8 Hz, 2H), 4.03 (s, 2H), 3.93-3.88 (m, 1H), 3.86-3.80 (m, 4H), 3.75-3.66 (m, 3H), 3.58-3.53 (m, 1H), 3.38 (dd, J=8.8 Hz & 1.6 Hz, 1H), 2.76 (dd, J=12.4 Hz & 4.0 Hz, 1H), 2.63 (td, J=6.8 Hz & 2.8 Hz, 2H), 2.52-2.48 (2H), 2.32 (t, J=2.8 Hz, 1H), 2.10-1.96 (m, 2H), 1.75 (t, J=12.4 Hz, 1H).

### (2R,4S,5R,6R)-2-((4-(but-3-yn-1-yloxy)benzyl)oxy)-6-((1R,2R)-1,2-dihydroxy-3-(2-(2-morpholinophenyl)acetamido)propyl)-4-hydroxy-5-(2-hydroxyacetamido)tetrahydro-2H-pyran-2-carboxylic acid (Compound 138)

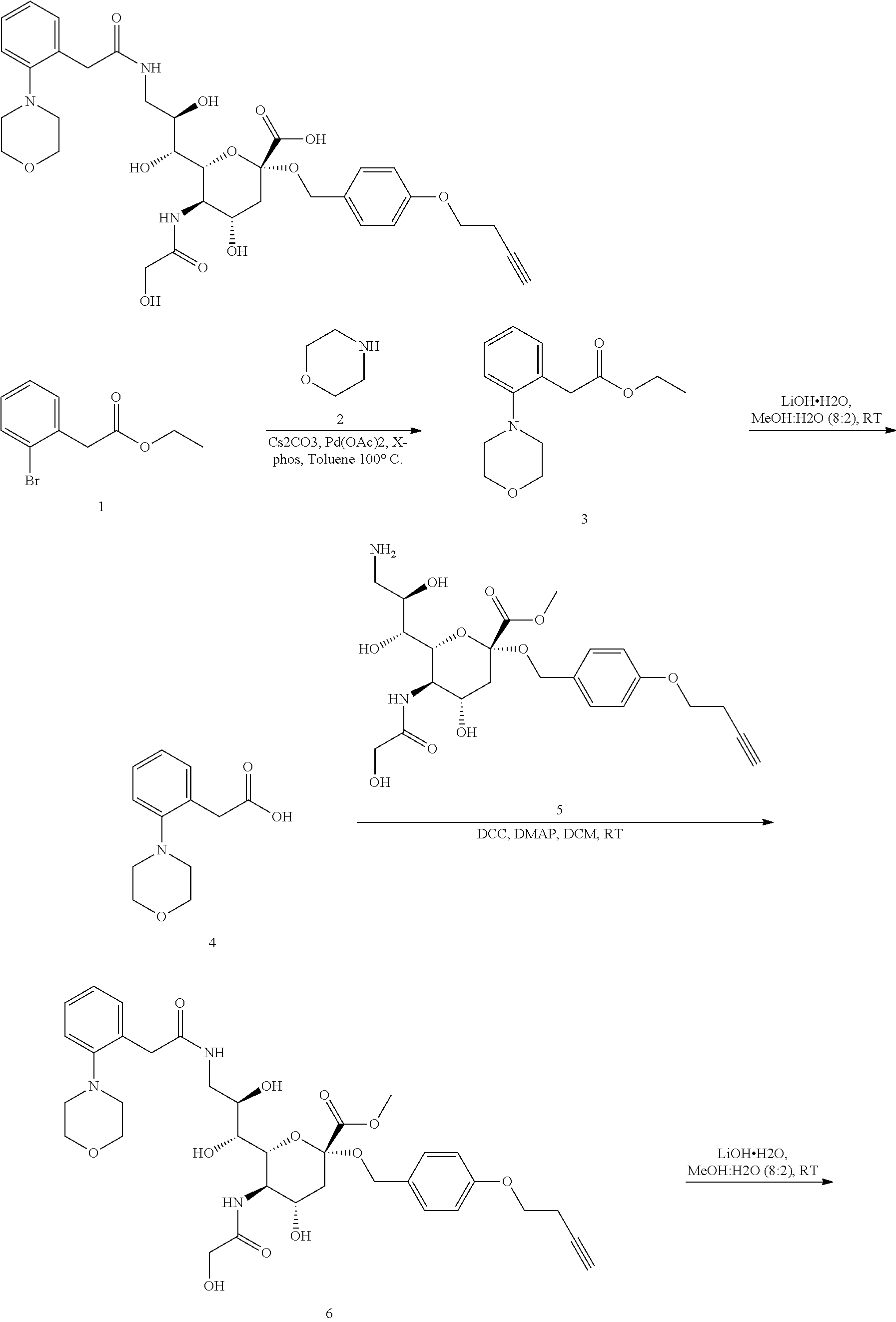

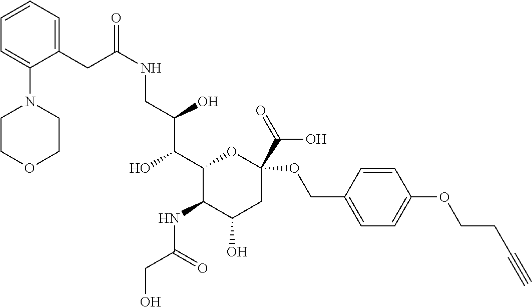 [SMILES: C#CCCOc1ccc(CO[C@]2(C(=O)O)C[C@H](O)[C@@H](NC(=O)CO)[C@H]([C@H](O)[C@H](O)CNC(=O)Cc3ccccc3N3CCOCC3)O2)cc1]

### Synthesis of ethyl 2-(2-morpholinophenyl)acetate (3)

To a stirred solution of ethyl 2-(4-bromophenyl)acetate (1, 2.0 g, 8.23 mmol) in toluene (20 mL), were added dicesium(1+) carbonate (5.36 g, 16.5 mmol), (1.0 g, 5.49 mmol) and morpholine (2, 1.42 mL, 16.5 mmol). The reaction mixture was degassed for 10 min by bubbling nitrogen. Palladium(2+) diacetate (0.092 g, 0.411 mmol) and dicyclohexyl[2′,4′,6′-tris(propan-2-yl)-[1,1′-biphenyl]-2-yl]phosphane (0.392 g, 0.823 mmol) were added was heated at 100° C. for 12 h. After completion, the reaction mixture was quenched with water and extracted with ethyl acetate. The organic layer was dried, filtered, and the filtrate was concentrated. The crude residue was purified by silica gel column chromatography using ethyl acetate in heptane as eluents to afford the desired product (3). Yield: 0.70 g, 35%; LCMS (ESI) m/z 250.20 [M+H]⁺.

### Synthesis of 2-(2-morpholinophenyl)acetic acid (4)

To a stirred solution of ethyl 2-(2-morpholinophenyl)acetate (3, 0.60 g, 2.41 mmol) in methanol (6.0 mL) was added a solution of lithium hydroxide monohydrate (0.156 g, 6.50 mmol) in water (1.0 mL). The reaction mixture was stirred at room temperature for 5 h. After completion, the reaction mixture was concentrated, and the mixture was treated with a solution of citric acid and extracted with ethyl acetate. The organic layer was dried, filtered, and the filtrate was concentrated to afford the desired product (4). Yield: 0.250 g, 47%; LCMS (ESI) m/z 222.10 [M+H]⁺.

### Synthesis of methyl (2R,4S,5R,6R)-2-((4-(but-3-yn-1-yloxy)benzyl)oxy)-6-((1R,2R)-1,2-dihydroxy-3-(2-(2-morpholinophenyl)acetamido)propyl)-4-hydroxy-5-(2-hydroxyacetamido)tetrahydro-2H-pyran-2-carboxylate (6)

To a stirred solution of 2-(2-morpholinophenyl)acetic acid (4, 0.120 g, 0.542 mmol) in dichloromethane (7.0 mL), were added N,N′-Dicyclohexylcarbodiimide (0.223 g, 1.084 mmol), N,N-dimethylpyridin-4-amine (0.066 g, 0.542 mmol) and methyl (2R,4S,5R,6R)-6-((1R,2R)-3-amino-1,2-dihydroxypropyl)-2-((4-(but-3-yn-1-yloxy)benzyl)oxy)-4-hydroxy-5-(2-hydroxyacetamido)tetrahydro-2H-pyran-2-carboxylate (0.269 g, 0.542 mmol) at 0° C. After 3 h at room temperature the reaction mixture was concentrated to afford the desired product (6). Yield: 0.30 g, 79%; LCMS m/z 700.35 [M+H]⁺.

### Synthesis of (2R,4S,5R,6R)-2-((4-(but-3-yn-1-yloxy)benzyl)oxy)-6-((1R,2R)-1,2-dihydroxy-3-(2-(2-morpholinophenyl)acetamido)propyl)-4-hydroxy-5-(2-hydroxyacetamido)tetrahydro-2H-pyran-2-carboxylic acid (Cpd. 138)

To a stirred solution of methyl (2R,4S,5R,6R)-2-((4-(but-3-yn-1-yloxy)benzyl)oxy)-6-((1R,2R)-1,2-dihydroxy-3-(2-(2-morpholinophenyl)acetamido)propyl)-4-hydroxy-5-(2-hydroxyacetamido)tetrahydro-2H-pyran-2-carboxylate (6, 0.30 g, 0.428 mmol) in methanol (5.0 mL) was added a solution of lithium hydroxide monohydrate (0.092 g, 2.14 mmol) in water (1.0 mL). The reaction mixture was stirred at room temperature for 4 h. After completion, the reaction mixture was treated with acidic resin (Dowex 50, H⁺) to pH˜6 and the suspension was filtered. The filtrate was concentrated, and the crude material was purified with preparative HPLC (15-40% water and acetonitrile (+0.1% TFA) as eluents to afford the desired product (Cpd. 138). Yield: 0.021 g, 7%; LCMS (ESI) m/z 686.4 [M+H]⁺. ¹H NMR (400 MHz, methanol-d₄) δ 7.36-7.30 (m, 3H), 7.25-7.21 (m, 3H), 6.90-6.87 (m, 2H), 4.72 (d, J=8.4 HZ, 1H), 4.38 (d, J=10.8 HZ, 1H), 4.07 (t, J=6.8 Hz, 2H), 4.0 (s, 2H), 3.93-3.89 (m, 5H), 3.87-3.74 (m, 6H), 3.40-3.37 (m, 1H), 3.26-3.09 (m, 3H), 2.76 (dd, J=12.8 & 4.4 Hz, 1H), 2.64 (td, J=6.8 & 2.8 Hz, 2H), 2.33 (t, J=2.8 Hz, 1H), 1.77 (t, J=12.8 Hz, 1H).

### (2R,4S,5R,6R)-6-((1R,2R)-3-(2-(3-fluoro-5-(trifluoromethyl)phenyl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 133)

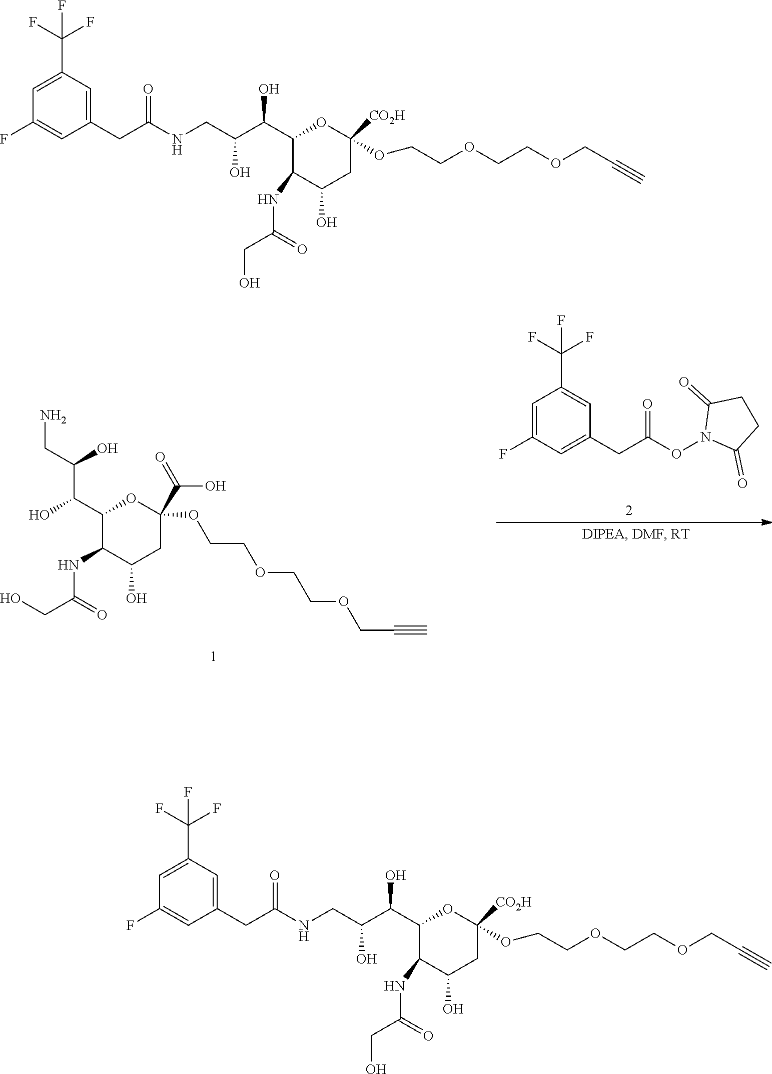 [SMILES: C#CCOCCOCCO[C@]1(C(=O)O)C[C@H](O)[C@@H](NC(=O)CO)[C@H]([C@H](O)[C@H](O)CNC(=O)Cc2cc(F)cc(C(F)(F)F)c2)O1 ; C#CCOCCOCCO[C@]1(C(=O)O)C[C@H](O)[C@@H](NC(=O)CO)[C@H]([C@H](O)[C@H](O)CN)O1 ; O=C(Cc1cc(F)cc(C(F)(F)F)c1)ON1C(=O)CCC1=O ; C#CCOCCOCCO[C@]1(C(=O)O)C[C@H](O)[C@@H](NC(=O)CO)[C@H]([C@H](O)[C@H](O)CNC(=O)Cc2cc(F)cc(C(F)(F)F)c2)O1]

### Synthesis of (2R,4S,5R,6R)-6-((1R,2R)-3-(2-(3-fluoro-5-(trifluoromethyl)phenyl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 133)

To a stirred solution of (2R,4S,5R,6R)-6-((1R,2R)-3-amino-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (1, 0.300 g, 0.666 mmol) and 2,5-dioxopyrrolidin-1-yl 2-(3-fluoro-5-(trifluoromethyl)phenyl)acetate (0.170 g, 0.532 mmol) in N,N-dimethylformamide (3.0 mL) was added ethylbis(propan-2-yl)amine (0.61 mL, 3.33 mmol) at 0° C. then stirred at room temperature for 4 h. After completion, the reaction mixture was concentrated. The crude was purified by prep HPLC using acetonitrile in water (+0.1% TFA) as eluents to afford the desired product (Cpd. 133). Yield: 0.035 g; 9%; LCMS (ESI) m/z 655.30 [M+H]⁺; ¹H NMR (400 MHz, MeOD-d₄): δ 8.20 (t, J=5.4 Hz, 1H), 7.90 (d, J=7.6 Hz, 1H), 7.48 (s, 1H), 7.38-7.32 (m, 2H), 4.18 (d, J=2.4 Hz, 1H), 4.01 (s, 2H), 3.93-3.78 (m, 4H), 3.73-3.57 (m, 11H), 3.37 (dd, J=8.8 Hz & 1.6 Hz, 1H), 3.25-3.19 (m, 1H), 2.83 (t, J=2.4 Hz, 1H), 2.74 (dd, J=12.4 Hz & 3.6 Hz, 1H), 1.74 (t, J=12.4 Hz, 1H).

### (2R,4S,5R,6R)-2-(3-amino-2-hydroxypropoxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Compound 104)

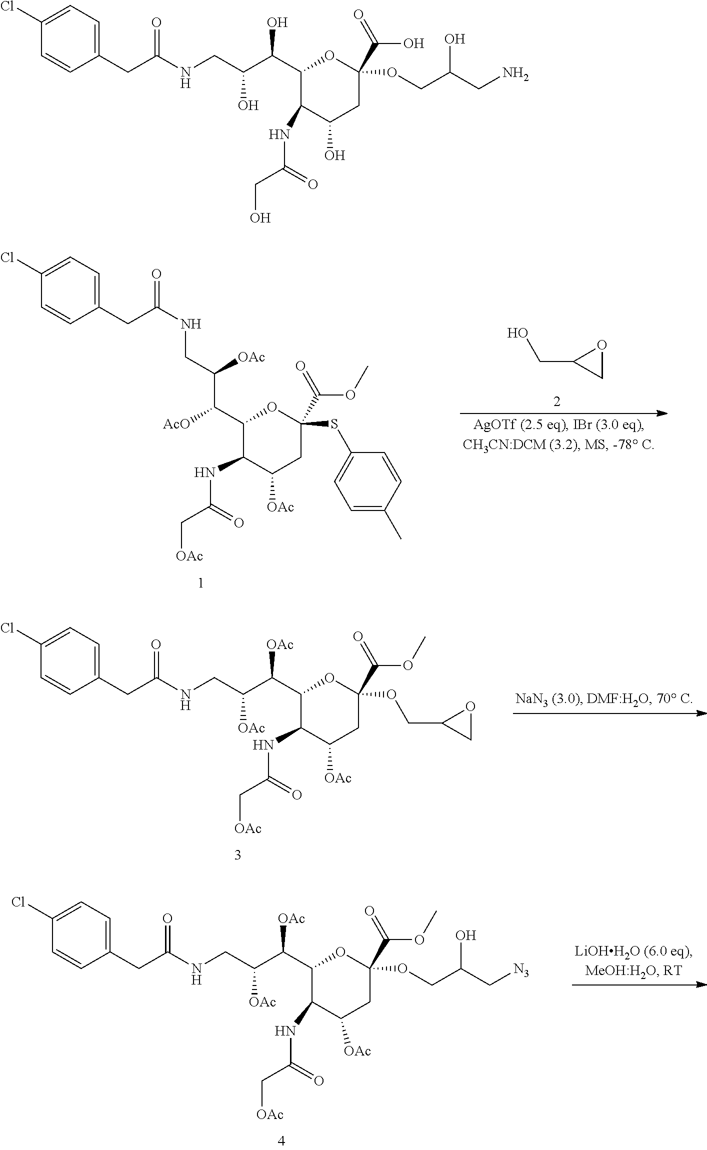 [SMILES: NCC(O)CO[C@]1(C(=O)O)C[C@H](O)[C@@H](NC(=O)CO)[C@H]([C@H](O)[C@H](O)CNC(=O)Cc2ccc(Cl)cc2)O1 ; COC(=O)[C@]1(Sc2ccc(C)cc2)C[C@H](OC(C)=O)[C@@H](NC(=O)COC(C)=O)[C@H]([C@H](OC(C)=O)[C@@H](CNC(=O)Cc2ccc(Cl)cc2)OC(C)=O)O1 ; OCC1CO1 ; COC(=O)[C@@]1(OCC2CO2)C[C@H](OC(C)=O)[C@@H](NC(=O)COC(C)=O)[C@H]([C@H](OC(C)=O)[C@@H](CNC(=O)Cc2ccc(Cl)cc2)OC(C)=O)O1 ; COC(=O)[C@@]1(OCC(O)CN=[N+]=[N-])C[C@H](OC(C)=O)[C@@H](NC(=O)COC(C)=O)[C@H]([C@H](OC(C)=O)[C@@H](CNC(=O)Cc2ccc(Cl)cc2)OC(C)=O)O1]

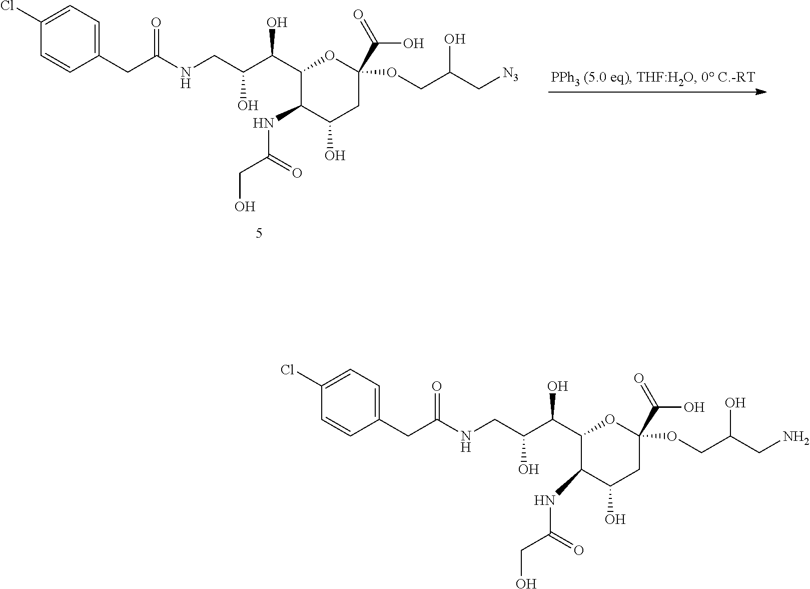 [SMILES: [N-]=[N+]=NCC(O)CO[C@]1(C(=O)O)C[C@H](O)[C@@H](NC(=O)CO)[C@H]([C@H](O)[C@H](O)CNC(=O)Cc2ccc(Cl)cc2)O1 ; NCC(O)CO[C@]1(C(=O)O)C[C@H](O)[C@@H](NC(=O)CO)[C@H]([C@H](O)[C@H](O)CNC(=O)Cc2ccc(Cl)cc2)O1]

### Synthesis of (1R,2R)-1-((2R,3R,4S,6R)-4-acetoxy-3-(2-acetoxyacetamido)-6-(methoxycarbonyl)-6-(oxiran-2-ylmethoxy)tetrahydro-2H-pyran-2-yl)-3-(2-(4-chlorophenyl)acetamido)propane-1,2-diyl diacetate (3)

A solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(3-acetoxyphenyl)acetamido)propane-1,2-diyl diacetate (1, 0.70 g, 0.915 mmol), (oxiran-2-yl)methanol (2, 0.339 g, 4.57 mmol), silver(1+) trifluoromethanesulfonate (0.705 g, 2.74 mmol) and activated 4 Å powdered molecular sieves (1.00 g) in anhydrous dichloromethane (16 mL) and anhydrous acetonitrile (25 mL) was stirred at room temperature for 1 h. The mixture was cooled to −78° C. and a solution of iodobromane (0.378 g, 1.83 mmol) in dichloromethane (5.0 mL) was added dropwise. The cooling bath was removed, and the mixture was stirred at this temperature for 2 h. After completion, the reaction mixture was quenched by triethylamine (2.0 mL) and warmed to room temperature. The reaction mixture was filtered and washed with dichloromethane. The filtrate was washed with saturated aqueous solution of sodium bicarbonate and the organic layer was dried, filtered, and concentrated. The crude residue obtained was purified by silica gel column chromatography using ethyl acetate in heptane as eluents to afford the desired product (3). Yield: 0.50 g, 76.4%; LCMS m/z 715.05 [M+H]⁺.

### Synthesis of (1R,2R)-1-((2R,3R,4S,6R)-4-acetoxy-3-(2-acetoxyacetamido)-6-(3-azido-2-hydroxypropoxy)-6-(methoxycarbonyl)tetrahydro-2H-pyran-2-yl)-3-(2-(4-chlorophenyl)acetamido)propane-1,2-diyl diacetate (4)

To a solution of methyl (2R,4S,5R,6R)-6-[(1R,2R)-1,2-diacetoxy-3-{1[(p-chlorophenyl)methyl]carbonylamino}propyl]-4-acetoxy-5-[(acetoxymethyl)carbonylamino]-2-[(2-oxiranyl)methoxy]tetrahydro-2H-pyran-2-carboxylate (3, 0.50 g, 0.70 mmol) in N,N-dimethylformamide (5 mL):water (0.5 mL), was added sodium azide (0.090 g, 1.44 mmol) was stirred at 50° C. for 12 h. After completion the solvent was concentrated, and the crude was used “as is” for next step without further purification. Yield: 0.10 g, Crude; LCMS m/z 758.05 [M+H]⁺.

### Synthesis of (2R,4S,5R,6R)-2-(3-azido-2-hydroxypropoxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (5)

To a solution of methyl (2R,4S,5R,6R)-6-[(1R,2R)-1,2-diacetoxy-3-1{[(p-chlorophenyl)methyl]carbonylamino}propyl]-4-acetoxy-5-[(acetoxymethyl)carbonylamino]-2-(3-azido-2-hydroxypropoxy)tetrahydro-2H-pyran-2-carboxylate (4, 0.40 g, 0.528 mmol) in methanol (5 mL), was added sodium methoxide (0.034.9 mL, 0.158 mmol) at 0° C. The reaction mixture was stirred at room temperature for 4 h. After completion, the reaction mixture was treated with acidic resin (Dowex 50H⁺) to pH˜6 and the suspension was filtered. The filtrate was concentrated, and the crude residue obtained was purified by prep HPLC using acetonitrile and water (+0.05% TFA) to afford the desired product (5). Yield: 0.051 g, 16.78%; LCMS m/z 576.2 [M+H]⁺.

### Synthesis of (2R,4S,5R,6R)-2-(3-amino-2-hydroxypropoxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Cpd. 104)

To a stirred solution of (2R,4S,5R,6R)-6-[(1R,2R)-3-{[(p-chlorophenyl)methyl]carbonylamino}-1,2-dihydroxypropyl]-2-(3-azido-2-hydroxypropoxy)-4-hydroxy-5-[(hydroxymethyl)carbonylamino]tetrahydro-2H-pyran-2-carboxylic acid (5, 0.049 g, 0.085 mmol) in tetrahydrofuran (2.0 mL) and water (8.0 mL) was added triphenylphosphine (0.032 g, 0.425 mmol) at 0° C. The reaction mixture was allowed to stir at room temperature for 12 h then concentrated. The residue obtained was dissolved in water and ethyl acetate. The aqueous layer was separated and concentrated. The crude residue obtained was purified by prep HPLC using in water and acetonitrile (+0.05% TFA) as eluents to afford the desired product (Cpd. 104). Yield: 0.007 g, 15.2%; LCMS m/z 550.40 [M+H]⁺; ¹H NMR (400 MHz, methanol-d₄): δ 7.32-7.27 (m, 4H), 4.02 (bs, 2H), 3.92-3.69 (m, 7H), 3.60-3.55 (m, 1H), 3.53 (s, 2H), 3.49-3.45 (m, 1H), 3.37-3.34 (m, 1H), 3.11-3.08 (m, 1H), 3.06-3.05 (m, 1H), 2.97-2.92 (m, 1H), 2.77-2.73 (dd, J=4.4 Hz, 12.0 Hz, 1H), 1.66 (t, J=12.0 Hz, 1H).

### (2R,4S,5R,6R)-6-((1R,2R)-1,2-dihydroxy-3-(2-(3-hydroxyphenyl)acetamido)propyl)-4-hydroxy-5-(2-hydroxyacetamido)-2-((6-(prop-2-yn-1-yloxy)hexyl)oxy)tetrahydro-2H-pyran-2-carboxylic acid (Compound 144)

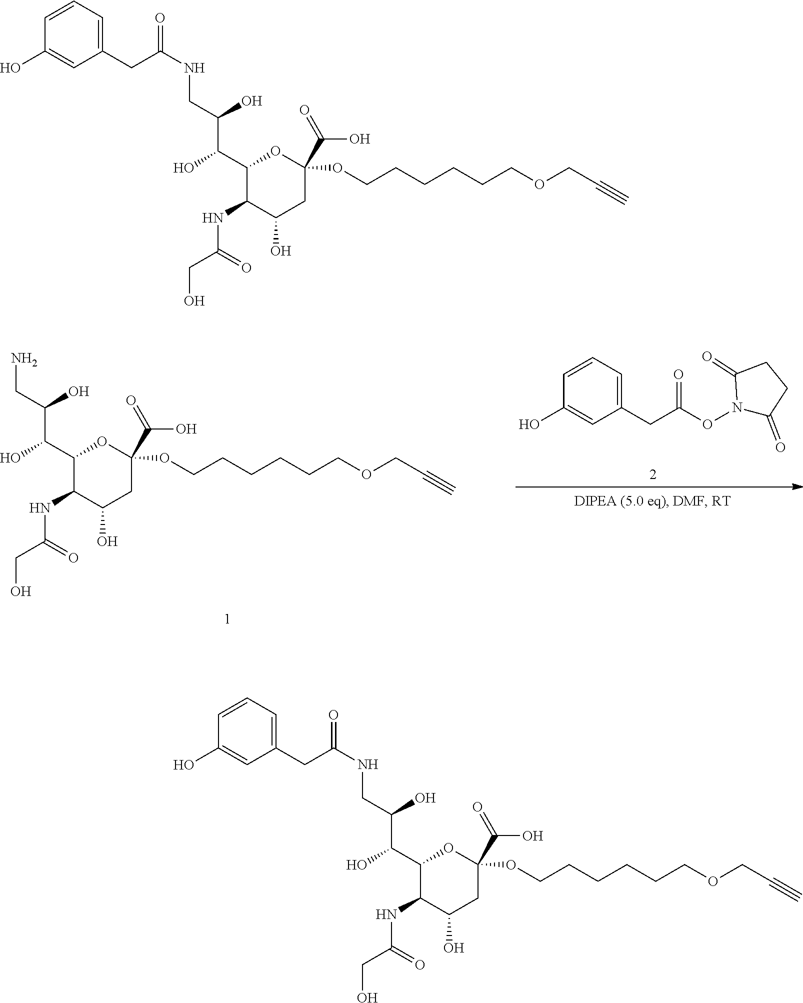 [SMILES: C#CCOCCCCCCO[C@]1(C(=O)O)C[C@H](O)[C@@H](NC(=O)CO)[C@H]([C@H](O)[C@H](O)CNC(=O)Cc2cccc(O)c2)O1 ; C#CCOCCCCCCO[C@]1(C(=O)O)C[C@H](O)[C@@H](NC(=O)CO)[C@H]([C@H](O)[C@H](O)CN)O1 ; O=C(Cc1cccc(O)c1)ON1C(=O)CCC1=O ; C#CCOCCCCCCO[C@]1(C(=O)O)C[C@H](O)[C@@H](NC(=O)CO)[C@H]([C@H](O)[C@H](O)CNC(=O)Cc2cccc(O)c2)O1]

### Synthesis of (2R,4S,5R,6R)-6-((1R,2R)-1,2-dihydroxy-3-(2-(3-hydroxyphenyl)acetamido)propyl)-4-hydroxy-5-(2-hydroxyacetamido)-2-((6-(prop-2-yn-1-yloxy)hexyl)oxy)tetrahydro-2H-pyran-2-carboxylic acid (Cpd. 144)

To a stirred solution of (2R,4S,5R,6R)-6-((1R,2R)-3-amino-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (1, 0.20 g, 0.432 mmol) and 2,5-dioxopyrrolidin-1-yl 2-(3-hydroxyphenyl)acetate (2, 0.108 g, 0.432 mmol) in N,N-dimethylformamide (4.0 mL) was added ethylbis(propan-2-yl)amine (0.37 mL, 2.16 mmol) at 0° C. then stirred at room temperature for 4 h. After completion, the reaction mixture was concentrated, and the crude residue obtained was purified by prep HPLC using acetonitrile in water (+0.1% TFA) as eluents to afford the desired product (Cpd.144). Yield: 0.030 g, 11%; LCMS m/z 597.50 [M+H]⁺; ¹H NMR (400 MHz, methanol-d4): δ 7.90-7.82 (m, 1H), 7.11 (t, J=7.6 Hz, 1H), 6.76-6.74 (m, 2H), 6.65 (dd, J=8.4 Hz & 2.0 Hz, 1H), 4.12 (d, J=2.4 Hz, 1H), 4.01 (s, 2H), 3.89-3.61 (m, 6H), 3.51 (t, J=6.4 Hz, 2H), 3.46 (bs, 2H), 3.42-3.36 (m, 2H), 3.31-3.25 (m, 1H), 2.81 (t, J=2.4 Hz, 1H), 2.71 (dd, J=4.0 Hz & 12.4 Hz, 1H), 1.72 (t, J=12.0 Hz, 1H), 1.57-1.55 (m, 4H), 1.38-1.36 (m, 4H).

### (2S,4S,5R,6R)-6-((1R,2R)-3-(2-(4-chlorophenyl)acetamido)-1,2-dihydroxypropyl)-4-hydroxy-2-(1-hydroxy-7-(prop-2-yn-1-yloxy)heptyl)-5-(2-hydroxyacetamido)tetrahydro-2H-pyran-2-carboxylic acid (Compound 103)

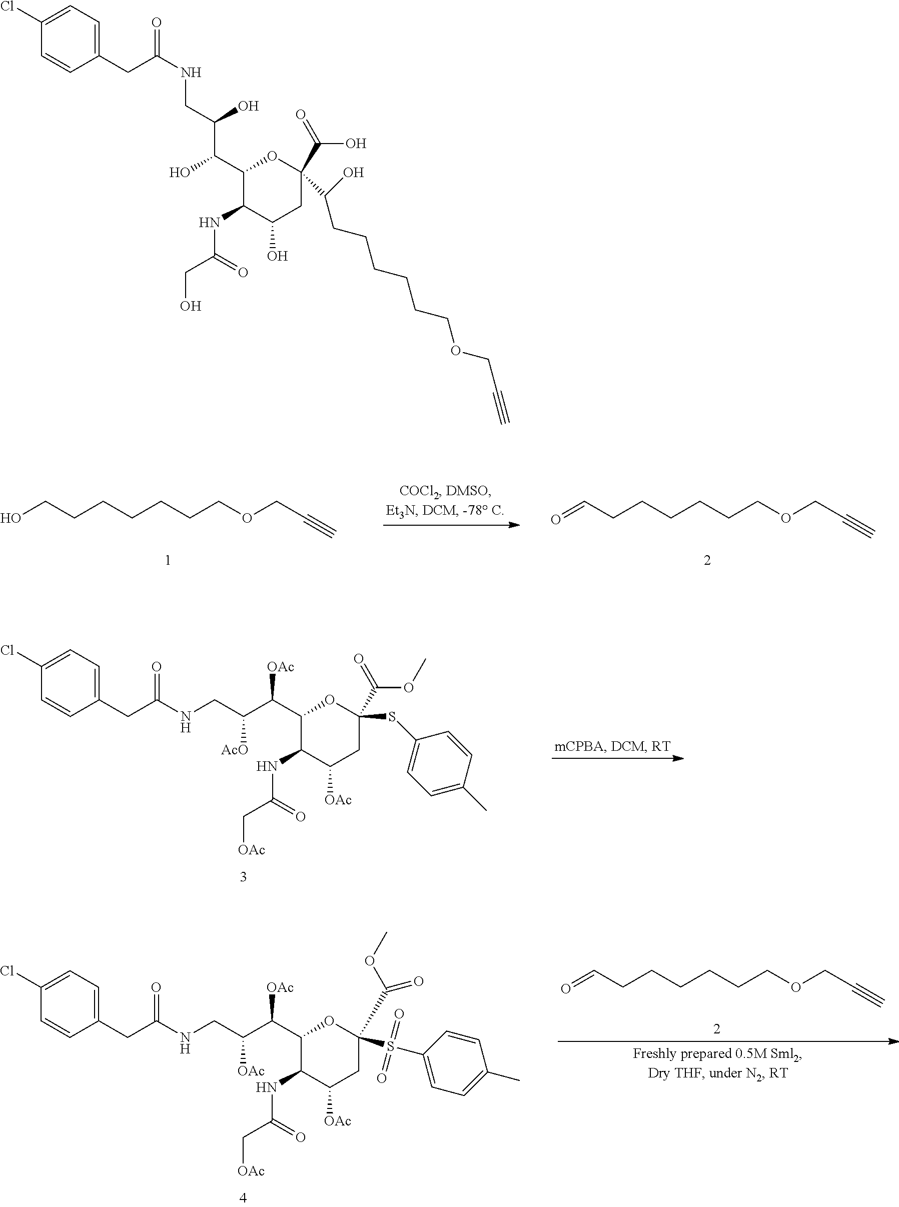 [SMILES: C#CCOCCCCCCC(O)[C@]1(C(=O)O)C[C@H](O)[C@@H](NC(=O)CO)[C@H]([C@H](O)[C@H](O)CNC(=O)Cc2ccc(Cl)cc2)O1 ; C#CCOCCCCCCCO ; C#CCOCCCCCCC=O ; COC(=O)[C@]1(Sc2ccc(C)cc2)C[C@H](OC(C)=O)[C@@H](NC(=O)COC(C)=O)[C@H]([C@H](OC(C)=O)[C@@H](CNC(=O)Cc2ccc(Cl)cc2)OC(C)=O)O1 ; COC(=O)[C@]1(S(=O)(=O)c2ccc(C)cc2)C[C@H](OC(C)=O)[C@@H](NC(=O)COC(C)=O)[C@H]([C@H](OC(C)=O)[C@@H](CNC(=O)Cc2ccc(Cl)cc2)OC(C)=O)O1 ; C#CCOCCCCCCC=O]

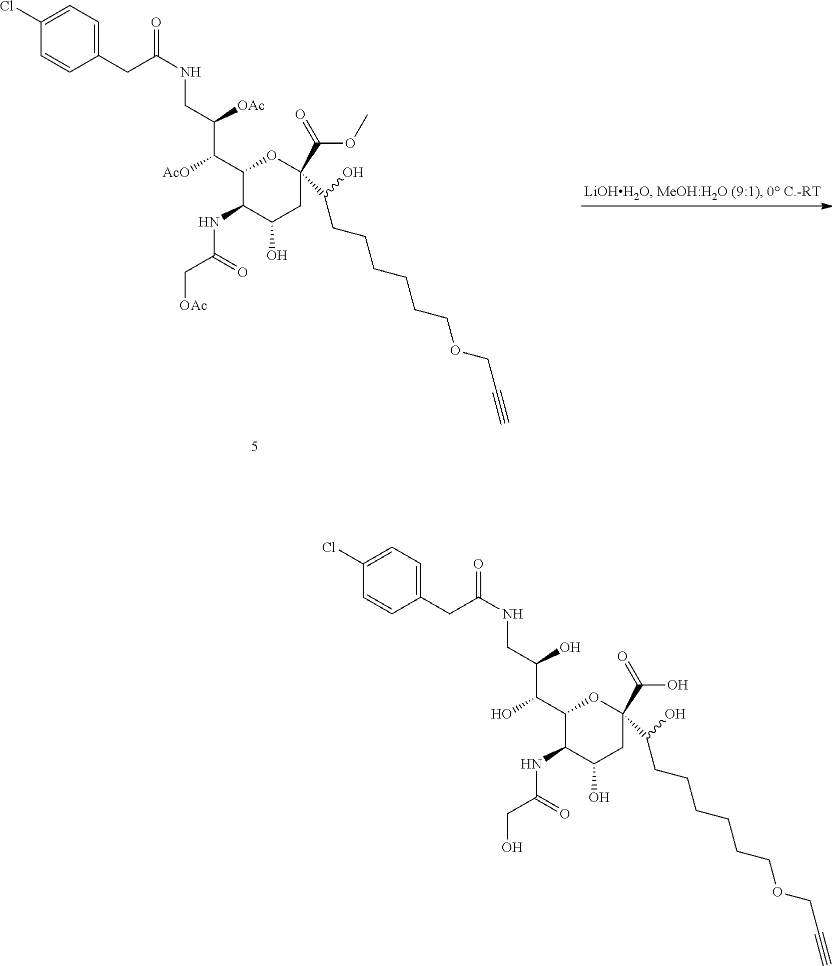 [SMILES: C#CCOCCCCCCC(O)[C@]1(C(=O)OC)C[C@H](O)[C@@H](NC(=O)COC(C)=O)[C@H]([C@H](OC(C)=O)[C@@H](CNC(=O)Cc2ccc(Cl)cc2)OC(C)=O)O1 ; C#CCOCCCCCCC(O)[C@]1(C(=O)O)C[C@H](O)[C@@H](NC(=O)CO)[C@H]([C@H](O)[C@H](O)CNC(=O)Cc2ccc(Cl)cc2)O1]

### Synthesis of 7-(prop-2-yn-1-yloxy)heptanal (2)

To a stirred solution of oxalic dichloride (1.51 mL, 17.6 mmol) in dichloromethane (50.0 ml) was added dropwise a solution of dimethylsulfoxide (2.09 mL, 29.4 mmol) in dichloromethane (2.0 mL) at −70° C. The mixture was stirred at −78° C. for 30 min, then 7-(prop-2-yn-1-yloxy)heptan-1-ol (1, 2.5 g, 14.7 mmol) in dichloromethane (5.0 mL) was added dropwise to the reaction mixture. The mixture was stirred at −78° C. for 90 min, then trimethylamine (10.2 mL, 73.4 mmol) was added. After completion, the reaction mixture was quenched with water (20 mL) at −78° C., then allowed to warm up to room temperature. The layers were separated and the aqueous layer was further extracted with dichloromethane (3×50 mL). The combined organic layers were dried and concentrated. The crude was purified by silica gel column chromatography, using ethyl acetate in heptane as eluents to afford the desired product (2). Yield: 2.1 g, 85%; ¹H NMR (400 MHz, DMSO-d₆): δ 9.65 (s, 1H), 4.09 (d, J=3.0 Hz, 2H), 3.42-3.39 (m, 2H), 2.43-2.39 (m, 1H), 1.53-1.45 (m, 5H), 1.28-1.27 (m, 5H).

### Synthesis of (1R,2R)-1-((2R,3R,4S,6R)-4-acetoxy-3-(2-acetoxyacetamido)-6-(methoxycarbonyl)-6-tosyltetrahydro-2H-pyran-2-yl)-3-(2-(4-chlorophenyl)acetamido)propane-1,2-diyl diacetate (4)

To a stirred solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (3, 1.0 g, 1.31 mmol) in dichloromethane (20 mL) was added m-chloroperbenzoic acid (1.13 g, 6.53 mmol) at 0° C. The resulting reaction mixture was stirred for 14 h at room temperature. After completion, the reaction mixture was diluted with dichloromethane and the organic layer was sequentially washed with sodium meta bisulphate and with saturated sodium bicarbonate solution (3×30 mL), dried, filtered, and concentrated. The crude residue was purified by silica gel column chromatography using methanol and dichloromethane as eluents to afford the desired product (4). Yield: 0.7 g, 67%; LCMS m/z 796.95 [M+H]⁺.

### Synthesis of (1R,2R)-1-((2R,3R,4S,6S)-4-acetoxy-3-(2-acetoxyacetamido)-6-(1-hydroxy-7-(prop-2-yn-1-yloxy)heptyl)-6-(methoxycarbonyl)tetrahydro-2H-pyran-2-yl)-3-(2-(4-chlorophenyl)acetamido)propane-1,2-diyl diacetate (5)

To a mixture of (1R,2R)-1-((2R,3R,4S,6R)-4-acetoxy-3-(2-acetoxyacetamido)-6-(methoxycarbonyl)-6-tosyltetrahydro-2H-pyran-2-yl)-3-(2-(4-chlorophenyl)acetamido)propane-1,2-diyl diacetate (4, 0.30 g, 0.37 mmol), 7-(prop-2-yn-1-yloxy)heptanal (2, 0.13 g, 0.75 mmol), was added freshly prepared samarium iodide solution (0.5 M in tetrahydrofuran, 30 mL) at room temperature was stirred for 10 min at room temperature. The reaction mixture was quenched with saturated aqueous solution of sodium thiosulfate pentahydrate (10 mL), filtered, and the filtrate was extracted with dichloromethane, dried, filtered, and the filtrate was concentrated. The crude residue obtained was purified by silica gel column chromatography using ethyl acetate in heptane as eluents to afford the desired product (5). Yield: 0.09 g, 29%; LCMS m/z 811.00 [M+H]⁺.

### Synthesis of (2S,4S,5R,6R)-6-((1R,2R)-3-(2-(4-chlorophenyl)acetamido)-1,2-dihydroxypropyl)-4-hydroxy-2-(1-hydroxy-7-(prop-2-yn-1-yloxy)heptyl)-5-(2-hydroxyacetamido)tetrahydro-2H-pyran-2-carboxylic acid (Cpd. 103)

To a stirred solution of (1R,2R)-1-((2R,3R,4S,6S)-4-acetoxy-3-(2-acetoxyacetamido)-6-((S)-1-hydroxy-7-(prop-2-yn-1-yloxy)heptyl)-6-(methoxycarbonyl)tetrahydro-2H-pyran-2-yl)-3-(2-(4-chlorophenyl)acetamido)propane-1,2-diyl diacetate (5, 0.09 g, 0.11 mmol) in methanol (5.0 mL) was added a solution of lithium hydroxide monohydrate (0.016 g, 0.66 mmol) in water (0.5 mL). The reaction mixture was stirred at room temperature for 4 h. After completion, the reaction mixture was treated with acidic resin (Dowex 50H+) to pH ˜6 and the suspension was filtered. The filtrate was concentrated, and the crude residue obtained was purified by prep HPLC using acetonitrile in water (+0.1% TFA) as eluents to afford the desired product (Cpd.103). Yield: 0.03 g, 43%; LCMS m/z 629.35 [M+H]⁺; ¹H NMR (400 MHz, methanol-d₄): δ 7.90-7.87 (m, 1H), 7.32-7.27 (m, 4H), 4.12 (d, J=2.4 Hz, 2H), 4.02 (s, 2H), 3.90-3.73 (m, 2H), 3.71-3.59 (m, 3H), 3.53-3.48 (m, 4H), 3.37-3.31 (m, 2H), 3.26-3.17 (m, 1H), 2.81 (t, J=2.4 Hz, 1H), 2.53-2.43 (m, 1H), 2.09 (t, J=12.4 Hz, 0.5H), 1.77 (t, J=12.4 Hz, 0.5H), 1.59-1.49 (m, 4H), 1.37-1.32 (m, 6H).

### (2R,4S,5R,6R)-6-((1R,2R)-3-(2-(4-chlorophenyl)acetamido)-1,2-dihydroxypropyl)-2-((6-((3,4-dioxo-2-(prop-2-yn-1-ylamino)cyclobut-1-en-1-yl)amino)hexyl)oxy)-4-hydroxy-5-(2-hydroxyacetamido)tetrahydro-2H-pyran-2-carboxylic acid (Compound 112)

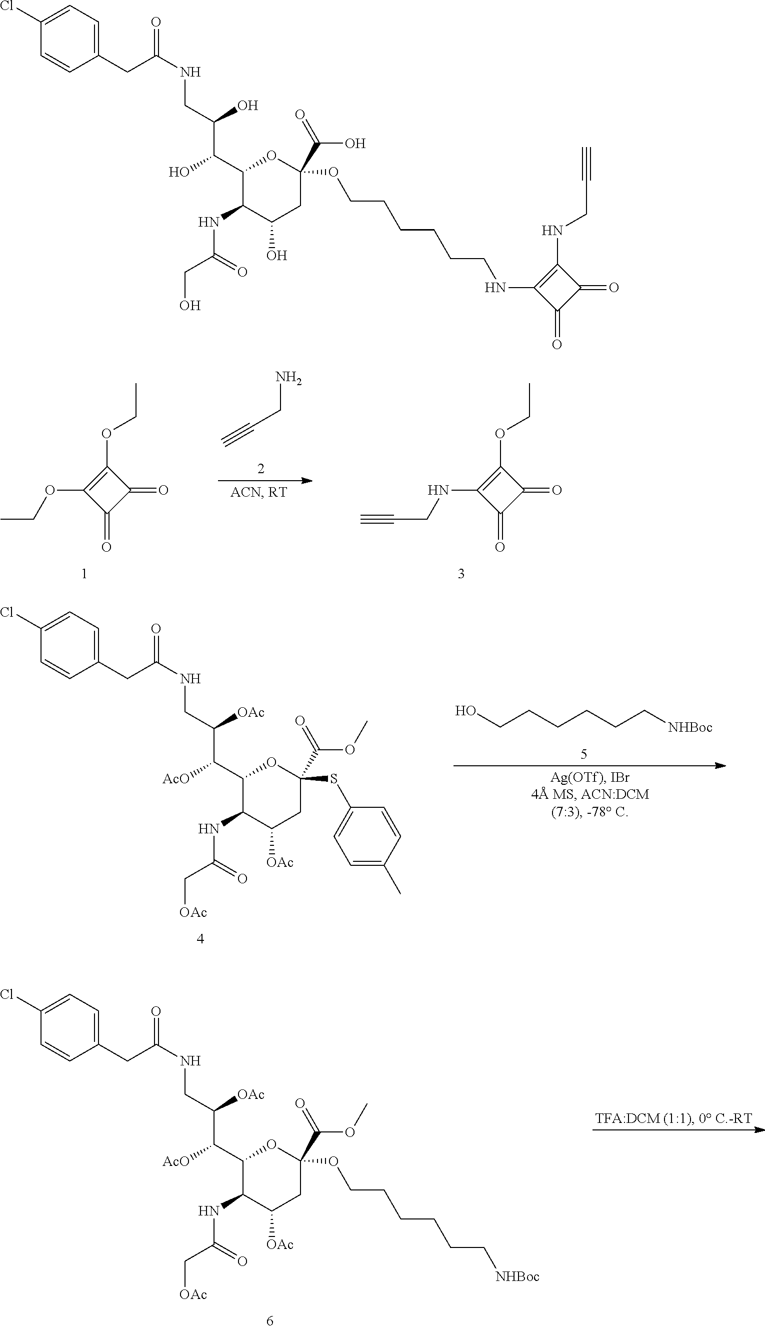

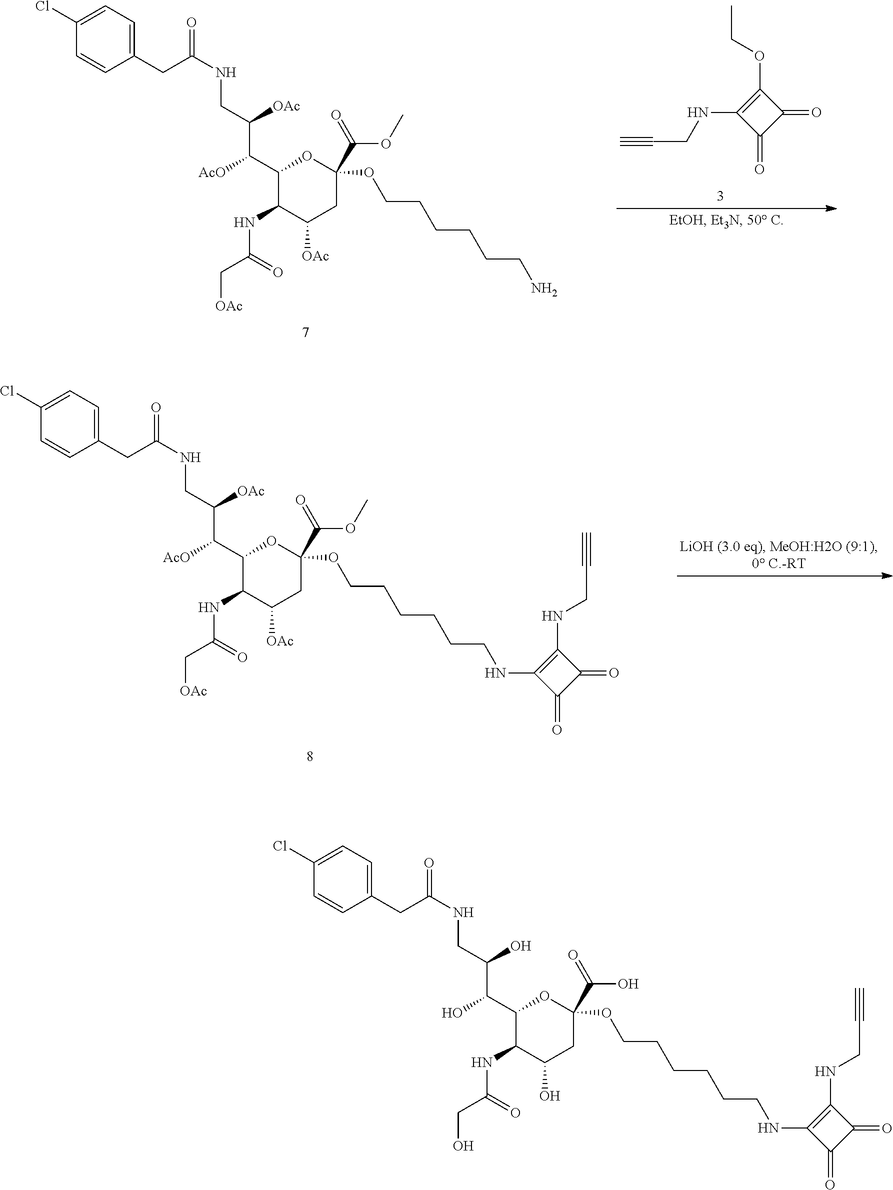 [SMILES: COC(=O)[C@@]1(OCCCCCCN)C[C@H](OC(C)=O)[C@@H](NC(=O)COC(C)=O)[C@H]([C@H](OC(C)=O)[C@@H](CNC(=O)Cc2ccc(Cl)cc2)OC(C)=O)O1 ; C#CCNc1c(OCC)c(=O)c1=O ; C#CCNc1c(NCCCCCCO[C@]2(C(=O)OC)C[C@H](OC(C)=O)[C@@H](NC(=O)COC(C)=O)[C@H]([C@H](OC(C)=O)[C@@H](CNC(=O)Cc3ccc(Cl)cc3)OC(C)=O)O2)c(=O)c1=O ; C#CCNc1c(NCCCCCCO[C@]2(C(=O)O)C[C@H](O)[C@@H](NC(=O)CO)[C@H]([C@H](O)[C@H](O)CNC(=O)Cc3ccc(Cl)cc3)O2)c(=O)c1=O]

### Synthesis of 3-ethoxy-4-(prop-2-yn-1-ylamino)cyclobut-3-ene-1,2-dione (3)

To a stirred solution of 3,4-diethoxycyclobut-3-ene-1,2-dione (1.0 g, 5.88 mmol) in ethanol was added propargylamine (0.3 mL, 5.88 mmol) at room temperature. After 10 min the reaction mixture was cooled down to 0° C. and triethylamine (4.1 mL, 29.4 mmol) was added dropwise. The resulting reaction mixture was stirred at room temperature for 4 h. After completion, the reaction mixture was concentrated to afford the desired crude product (3) which was used for the next step without further purification. Yield: 1.0 g, Crude; LCMS (ESI) m/z 180.10 [M+H]⁺.

### Synthesis of (1R,2R)-1-((2R,3R,4S,6R)-4-acetoxy-3-(2-acetoxyacetamido)-6-((6-((tert-butoxycarbonyl)amino)hexyl)oxy)-6-(methoxycarbonyl)tetrahydro-2H-pyran-2-yl)-3-(2-(4-chlorophenyl)acetamido)propane-1,2-diyl diacetate (6)

A solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (4, 0.20 g, 0.26 mmol) and tert-butyl (6-hydroxyhexyl)carbamate (5, 0.28 g, 1.31 mmol), silver(1+) trifluoromethanesulfonate (0.17 g, 0.65 mmol) and activated 4 Å powdered molecular sieves (1.00 g) in anhydrous dichloromethane (35 mL) and anhydrous acetonitrile (60 mL) was stirred at room temperature for 1 h. The solution was cooled to −78° C. and a solution of iodobromane (0.08 g, 0.39 mmol) in dichloromethane (5.0 mL) was added dropwise. After 2 h of stirring at this temperature the reaction mixture was quenched by addition of triethylamine (1.0 mL) and allowed to warm up to room temperature. The reaction mixture was filtered, and washed with dichloromethane. The filtrate was washed with saturated aqueous solution of sodium bicarbonate and dried, filtered, and concentrated. The crude residue obtained was purified by silica gel column chromatography using ethyl acetate in heptane as eluents to afford the desired product (6). Yield: 0.10 g, 44%; LCMS m/z 859.90 [M+H]⁺.

### Synthesis of (1R,2R)-1-((2R,3R,4S,6R)-4-acetoxy-3-(2-acetoxyacetamido)-6-((6-aminohexyl)oxy)-6-(methoxycarbonyl)tetrahydro-2H-pyran-2-yl)-3-(2-(4-chlorophenyl)acetamido)propane-1,2-diyl diacetate (7)

To a stirred solution of (1R,2R)-1-((2R,3R,4S,6R)-4-acetoxy-3-(2-acetoxyacetamido)-6-((6-((tert-butoxycarbonyl)amino)hexyl)oxy)-6-(methoxycarbonyl)tetrahydro-2H-pyran-2-yl)-3-(2-(4-chlorophenyl)acetamido)propane-1,2-diyl diacetate (6, 0.10 g, 0.12 mmol) in anhydrous dichloromethane (3.0 mL), was added trifluoroacetic acid (1.0 mL) at 0° C. The reaction mixture was stirred at room temperature for 3 h then concentrated to afford the desired product (7) as a crude which was used for the next step without further purification. Yield: 0.10 g, Crude; LCMS (ESI) m/z 758.90 [M+H]⁺.

### Synthesis of (1R,2R)-1-((2R,3R,4S,6R)-4-acetoxy-3-(2-acetoxyacetamido)-6-((6-((3,4-dioxo-2-(prop-2-yn-1-ylamino)cyclobut-1-en-1-yl)amino)hexyl)oxy)-6-(methoxycarbonyl)tetrahydro-2H-pyran-2-yl)-3-(2-(4-chlorophenyl)acetamido)propane-1,2-diyl diacetate (8)

To a stirred solution of methyl (1R,2R)-1-((2R,3R,4S,6R)-4-acetoxy-3-(2-acetoxyacetamido)-6-((6-aminohexyl)oxy)-6-(methoxycarbonyl)tetrahydro-2H-pyran-2-yl)-3-(2-(4-chlorophenyl)acetamido)propane-1,2-diyl diacetate (7, 0.25 g, 0.33 mmol) in ethanol (5 mL, 85.6 mmol), 3-ethoxy-4-[(prop-2-yn-1-yl)amino]cyclobut-3-ene-1,2-dione (3, 59.1 mg, 0.33 mmol) was added at room temperature. After 10 min triethylamine (0.23 mL, 1.67 mmol) was added dropwise at 0° C. The resulting reaction mixture was stirred at 50° C. for 3 h. After completion, the reaction mixture was concentrated to afford the desired crude product (8) which was used for the next step without purification. Yield: 0.10 g, Crude; LCMS (ESI) m/z 891.05 [M+H]⁺.

### Synthesis of (2R,4S,5R,6R)-6-((1R,2R)-3-(2-(4-chlorophenyl)acetamido)-1,2-dihydroxypropyl)-2-((6-((3,4-dioxo-2-(prop-2-yn-1-ylamino)cyclobut-1-en-1-yl)amino)hexyl)oxy)-4-hydroxy-5-(2-hydroxyacetamido)tetrahydro-2H-pyran-2-carboxylic acid (Cpd. 112)

To a stirred solution of (1R,2R)-1-((2R,3R,4S,6R)-4-acetoxy-3-(2-acetoxyacetamido)-6-((6-((3,4-dioxo-2-(prop-2-yn-1-ylamino)cyclobut-1-en-1-yl)amino)hexyl)oxy)-6-(methoxycarbonyl)tetrahydro-2H-pyran-2-yl)-3-(2-(4-chlorophenyl)acetamido)propane-1,2-diyl diacetate (8, 0.10 g, 0.13 mmol) in methanol (5 mL) was added a solution of lithium hydroxide monohydrate (0.019 g, 0.81 mmol) in water (0.5 mL). The reaction mixture was stirred at room temperature for 6 h. After completion, the reaction mixture was treated with acidic resin (Dowex 50, H⁺) to pH˜6 and the suspension was filtered and rinsed with methanol. The filtrate was concentrated, and the crude residue obtained was purified by prep HPLC using acetonitrile in water (+0.1% TFA) as eluents to afford the desired product (Cpd. 112). Yield: 0.019 g, 22%; LCMS m/z 709.4 [M+H]⁺; ¹H NMR (400 MHz, Methanol-d₄): δ 7.91 (d, J=8.0 Hz, 1H), 7.31-7.28 (m, 4H), 4.42 (s, 2H), 4.02 (s, 2H), 3.89-3.68 (m, 6H), 3.60-3.50 (m, 4H), 3.42-3.35 (m, 2H), 3.22-3.18 (m, 1H), 2.83 (t, J=2.4 Hz, 1H), 2.70 (dd, J=12.4 Hz & 4.0 Hz, 1H), 1.72 (t, J=12.4 Hz, 3H), 1.60-1.55 (m, 4H), 1.39-1.33 (m, 4H).

### (2R,4S,5R,6R)-6-((1R,2R)-3-(2-(4-chlorophenyl)acetamido)-1,2-dihydroxypropyl)-2-((6-((3,4-dioxo-2-(((1-(15-oxo-15-(perfluorophenoxy)-3,6,9,12-tetraoxapentadecyl)-1H-1,2,3-triazol-4-yl)methyl)amino)cyclobut-1-en-1-yl)amino)hexyl)oxy)-4-hydroxy-5-(2-hydroxyacetamido)tetrahydro-2H-pyran-2-carboxylic acid (Compound 112P)

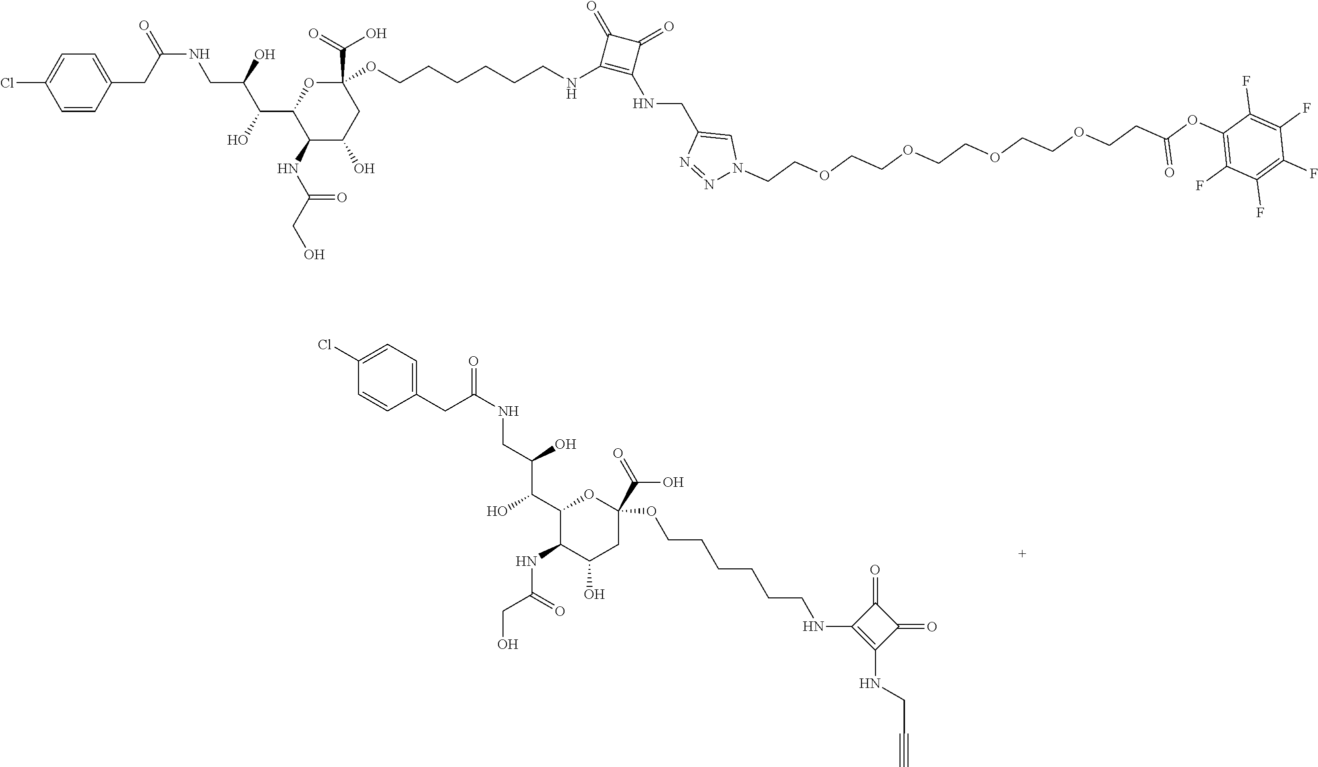 [SMILES: O=C(Cc1ccc(Cl)cc1)NC[C@@H](O)[C@@H](O)[C@@H]1O[C@@](OCCCCCCNc2c(NCc3cn(CCOCCOCCOCCOCCC(=O)Oc4c(F)c(F)c(F)c(F)c4F)nn3)c(=O)c2=O)(C(=O)O)C[C@H](O)[C@H]1NC(=O)CO ; C#CCNc1c(NCCCCCCO[C@]2(C(=O)O)C[C@H](O)[C@@H](NC(=O)CO)[C@H]([C@H](O)[C@H](O)CNC(=O)Cc3ccc(Cl)cc3)O2)c(=O)c1=O]

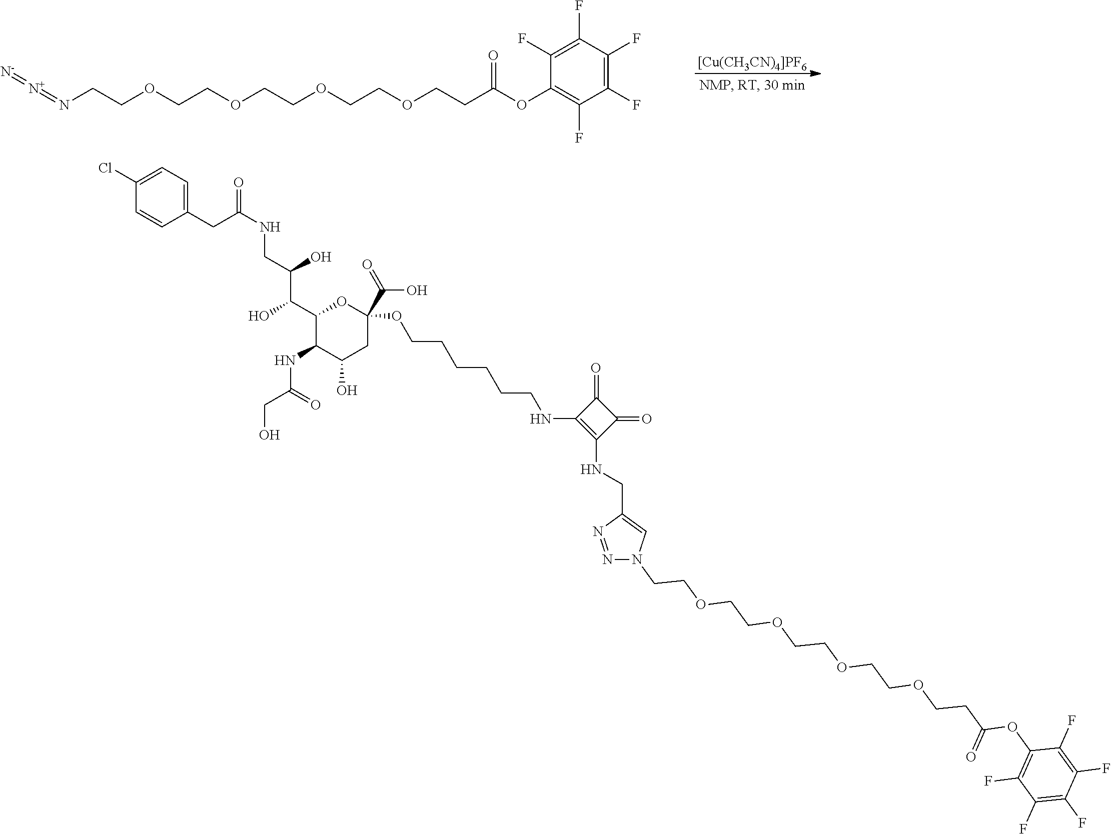

### Synthesis of (2R,4S,5R,6R)-6-((1R,2R)-3-(2-(4-chlorophenyl)acetamido)-1,2-dihydroxypropyl)-2-((6-((3,4-dioxo-2-(((1-(15-oxo-15-(perfluorophenoxy)-3,6,9,12-tetraoxapentadecyl)-1H-1,2,3-triazol-4-yl)methyl)amino)cyclobut-1-en-1-yl)amino)hexyl)oxy)-4-hydroxy-5-(2-hydroxyacetamido)tetrahydro-2H-pyran-2-carboxylic acid (112P)

To a solution of Azido-PEG4-PFP-ester (14.8 mg, 32.4 umol) in anhydrous NMP (190 uL) was added (2R,4S,5R,6R)-6-((1R,2R)-3-(2-(4-chlorophenyl)acetamido)-1,2-dihydroxypropyl)-2-((6-((3,4-dioxo-2-(prop-2-yn-1-ylamino)cyclobut-1-en-1-yl)amino)hexyl)oxy)-4-hydroxy-5-(2-hydroxyacetamido)tetrahydro-2H-pyran-2-carboxylic acid (112, 20.5 mg, 28.9 umol) in anhydrous NMP (190 uL) dropwise, followed by tetrakis(acetonitrile)copper(1) hexafluorophosphate (16.2 mg, 43.4 umol). The vial was sealed and the mixture was stirred for 30 min at room temperature. The mixture was filtered through a 0.2 μm PTFE filter, a few drops of acetic acid were added, and the filtrate was purified by C₁₈ reverse phase column chromatography using acetonitrile and water (+0.1% TFA) as eluents. The fractions containing the desired product were lyophilized to afford the desired product (112P) as a white solid. Yield: 16.9 mg, 50%; LCMS (ESI) m/z 1166 [M+H]⁺. ¹H NMR (500 MHz, DMSO-d₆): 8.00 (s, br, 1H), 7.99 (m, br, 1H), 7.85 (d, J=7.5 Hz, 1H), 7.32 (d, 8.5 Hz, 2H), 7.26 (d, J=8.5 Hz, 2H), 4.76 (s, br, 2H), 4.51 (t, J=5.0 Hz, 2H), 3.90-3.83 (m, 2H), 3.79 (t, J=5.0 Hz, 2H), 3.75 (t, J=6.0 Hz, 2H), 3.67-3.59 (m, 2H), 3.55-3.53 (m, 4H), 3.51-3.49 (m, 4H), 3.47-3.45 (m, 6H), 3.44 (s, 2H), 3.34 (m, br, 6H), 3.20 (d, J=9.0 Hz, 1H), 3.01 (t, J=6.0 Hz, 2H), 2.96-2.91 (m, 1H), 1.54-1.48 (m, 1H), 1.49-1.45 (m, 4H), 1.26 (m, br, 4H).

### (2R,4S,5R,6R)-6-((1R,2R)-3-(2-(4-chlorophenyl)acetamido)-1,2-dihydroxypropyl)-2-((6-((2-(((1-(18-(2,5-dioxo-2,5-dihydro-1H-pyrrol-1-yl)-15-oxo-3,6,9,12-tetraoxa-16-azaoctadecyl)-1H-1,2,3-triazol-4-yl)methyl)amino)-3,4-dioxocyclobut-1-en-1-yl)amino)hexyl)oxy)-4-hydroxy-5-(2-hydroxyacetamido)tetrahydro-2H-pyran-2-carboxylic acid (Cpd. 112M)

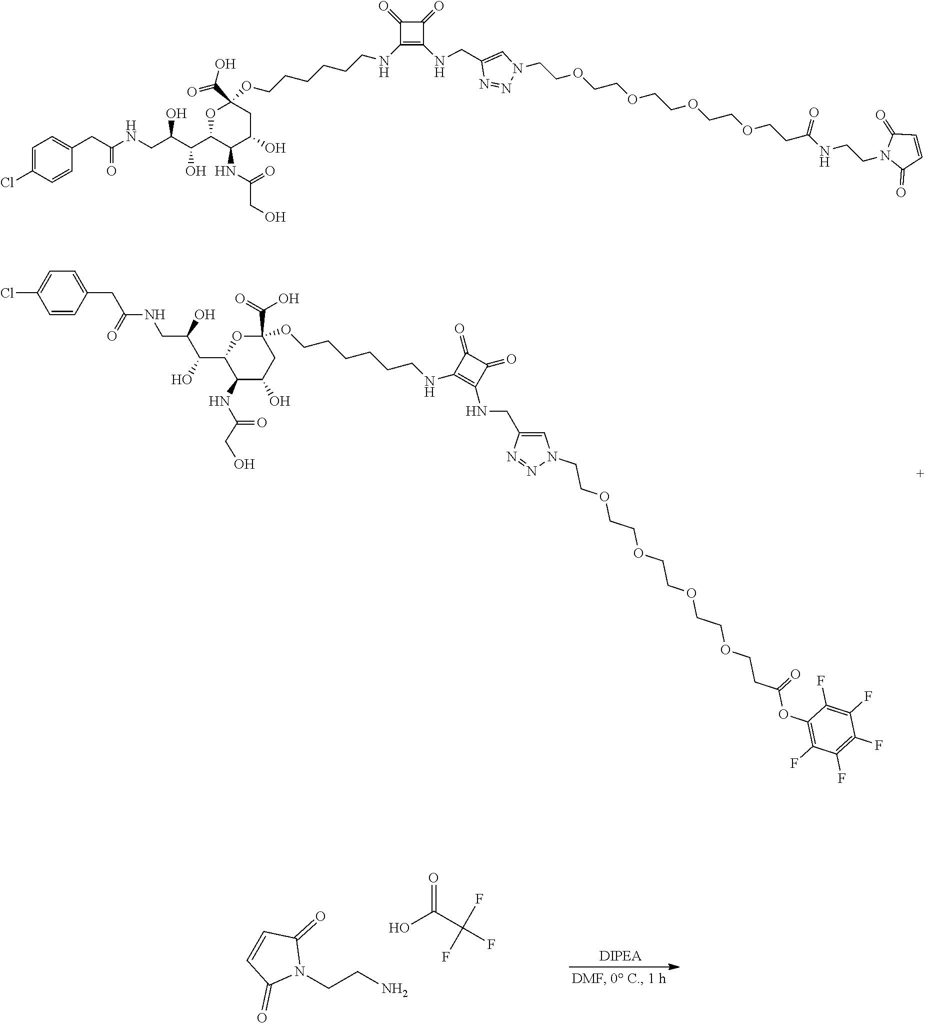 [SMILES: O=C(CCOCCOCCOCCOCCn1cc(CNc2c(NCCCCCCO[C@]3(C(=O)O)C[C@H](O)[C@@H](NC(=O)CO)[C@H]([C@H](O)[C@H](O)CNC(=O)Cc4ccc(Cl)cc4)O3)c(=O)c2=O)nn1)NCCN1C(=O)C=CC1=O ; O=C(Cc1ccc(Cl)cc1)NC[C@@H](O)[C@@H](O)[C@@H]1O[C@@](OCCCCCCNc2c(NCc3cn(CCOCCOCCOCCOCCC(=O)Oc4c(F)c(F)c(F)c(F)c4F)nn3)c(=O)c2=O)(C(=O)O)C[C@H](O)[C@H]1NC(=O)CO ; NCCN1C(=O)C=CC1=O ; O=C(O)C(F)(F)F]

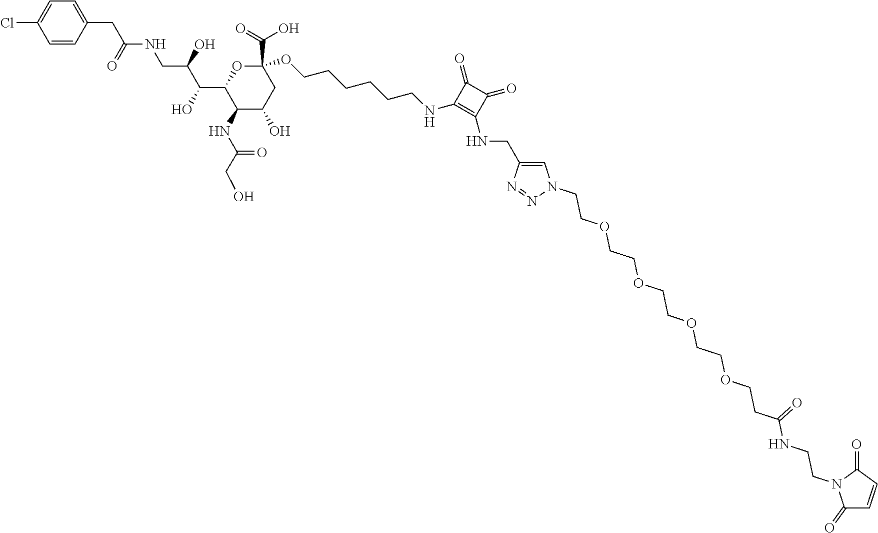 [SMILES: O=C(CCOCCOCCOCCOCCn1cc(CNc2c(NCCCCCCO[C@]3(C(=O)O)C[C@H](O)[C@@H](NC(=O)CO)[C@H]([C@H](O)[C@H](O)CNC(=O)Cc4ccc(Cl)cc4)O3)c(=O)c2=O)nn1)NCCN1C(=O)C=CC1=O]

### Synthesis of (2R,4S,5R,6R)-6-((1R,2R)-3-(2-(4-chlorophenyl)acetamido)-1,2-dihydroxypropyl)-2-((6-((2-(((1-(18-(2,5-dioxo-2,5-dihydro-1H-pyrrol-1-yl)-15-oxo-3,6,9,12-tetraoxa-16-azaoctadecyl)-1H-1,2,3-triazol-4-yl)methyl)amino)-3,4-dioxocyclobut-1-en-1-yl)amino)hexyl)oxy)-4-hydroxy-5-(2-hydroxyacetamido)tetrahydro-2H-pyran-2-carboxylic acid (112M)

To a solution of (2R,4S,5R,6R)-6-((1R,2R)-3-(2-(4-chlorophenyl)acetamido)-1,2-dihydroxypropyl)-2-((6-((3,4-dioxo-2-(((1-(15-oxo-15-(perfluorophenoxy)-3,6,9,12-tetraoxapentadecyl)-1H-1,2,3-triazol-4-yl)methyl)amino)cyclobut-1-en-1-yl)amino)hexyl)oxy)-4-hydroxy-5-(2-hydroxyacetamido)tetrahydro-2H-pyran-2-carboxylic acid (112P, 49.2 mg, 42.2 umol) in DMF (1.05 mL) was added n-(2-aminoethyl)maleimide trifluoroacetate salt (11.3 mg, 44.3 umol). The mixture was cooled down to 0° C. and stirred for 10 min. DIEA (22.0 uL, 127 umol) was added and the mixture was stirred for 1 h at 0° C. The reaction mixture was filtered and purified by preparatory HPLC using acetonitrile and water (+0.05% TFA) as eluents. The fractions containing the desired product were combined and lyophilized to dryness to afford the desired product (112M) as a white solid. Yield: 42.3 mg, 89%; LCMS (ESI) m/z 1122 [M+H]⁺. ¹H NMR (500 MHz, DMSO-d₆): 8.00 (s, br, 1H), 7.99 (m, br, 1H), 7.85 (d, J=7.5 Hz, 1H), 7.32 (d, J=8.5 Hz, 2H), 7.26 (d, J=8.5 Hz, 2H), 6.99 (s, 2H), 4.77 (s, br, 2H), 4.51 (t, J=5.0 Hz, 2H), 3.90-3.83 (m, 2H), 3.81 (m, 1H), 3.79 (t, J=5.0 Hz, 2H), 3.67-3.59 (m, 4H), 3.55-3.50 (m, 8H), 3.46 (m, 4H), 3.44 (s, 2H), 3.41 (m, br, 6H), 3.30-3.26 (m, 2H), 3.21-3.16 (m, 4H), 2.96-2.91 (m, 1H), 2.21 (t, J=7.0 Hz, 2H), 1.54-1.49 (m, 1H), 1.49-1.44 (m, 4H), 1.27 (m, br, 4H).

### (2R,4S,5R,6R)-6-((1R,2R)-3-(2-(4-fluoro-3-(trifluoromethyl)phenyl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 132)

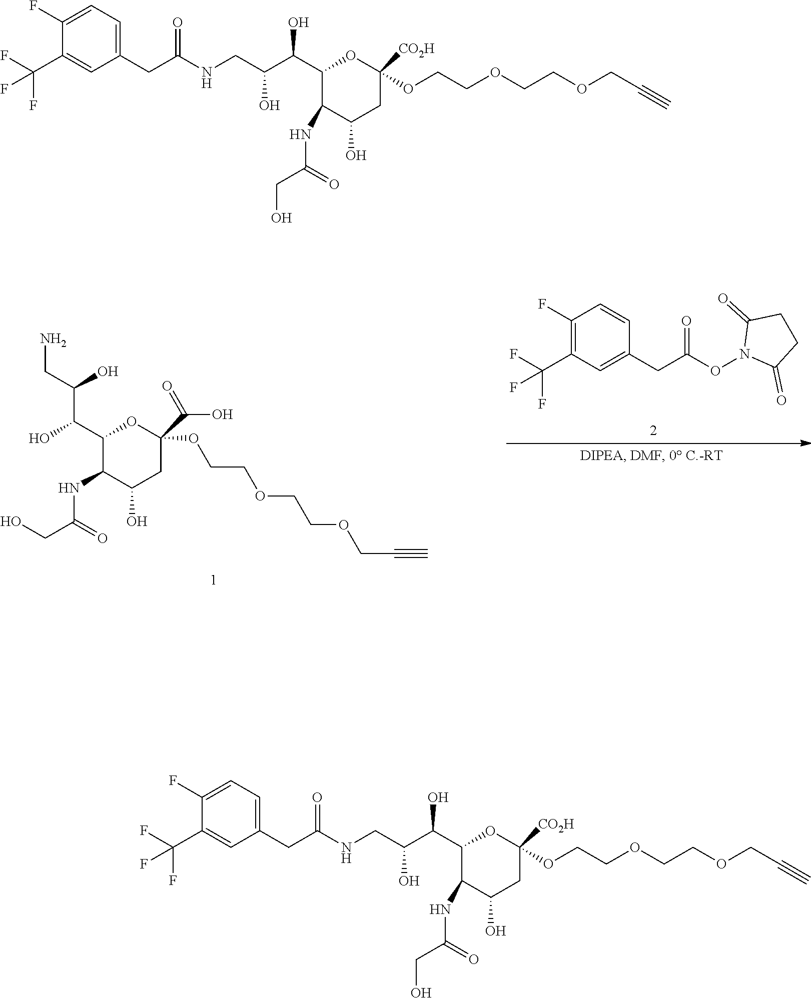 [SMILES: C#CCOCCOCCO[C@]1(C(=O)O)C[C@H](O)[C@@H](NC(=O)CO)[C@H]([C@H](O)[C@H](O)CNC(=O)Cc2ccc(F)c(C(F)(F)F)c2)O1 ; C#CCOCCOCCO[C@]1(C(=O)O)C[C@H](O)[C@@H](NC(=O)CO)[C@H]([C@H](O)[C@H](O)CN)O1 ; O=C(Cc1ccc(F)c(C(F)(F)F)c1)ON1C(=O)CCC1=O ; C#CCOCCOCCO[C@]1(C(=O)O)C[C@H](O)[C@@H](NC(=O)CO)[C@H]([C@H](O)[C@H](O)CNC(=O)Cc2ccc(F)c(C(F)(F)F)c2)O1]

### Synthesis of (2R,4S,5R,6R)-6-((1R,2R)-3-(2-(4-fluoro-3-(trifluoromethyl)phenyl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 132)

To a stirred solution of (2R,4S,5R,6R)-6-((1R,2R)-3-amino-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (1, 0.200 g, 0.44 mmol) and 2,5-dioxopyrrolidin-1-yl 2-(4-fluoro-3-(trifluoromethyl)phenyl)acetate (0.113 g, 0.355 mmol) in N,N-dimethylformamide (3.0 mL) was added ethylbis(propan-2-yl)amine (0.38 mL, 2.22 mmol) at 0° C. then stirred at room temperature for 4 h. After completion, the reaction mixture was concentrated, and the crude residue obtained was purified by prep HPLC using acetonitrile in water (+0.1% TFA) as eluents to afford the desired product (Cpd. 132). Yield: 0.025 g; 9%; LCMS (ESI) m/z 655.40 [M+H]⁺. ¹H NMR (400 MHz, MeOD-d4): δ 8.14 (m, 1H), 7.90 (d, J=8.0 Hz, 1H), 7.64 (d, J=6.4 Hz, 1H), 7.61-7.58 (m, 1H), 7.29-7.24 (m, 1H), 4.18 (d, J=2.4 Hz, 2H), 4.01 (s, 2H), 3.95-3.77 (m, 4H), 3.72-3.57 (m, 11H), 3.36 (dd, J=1.6 Hz, 1H), 3.24-3.19 (1H), 2.84 (t, J=5.6 Hz, 1H), 2.75 (dd, J=12.8 & 4.0 Hz, 1H), 1.73 (t, J=12.4 Hz, 1H).

### (2R,4S,5R,6R)-6-((1R,2R)-3-amino-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (Compound 16)

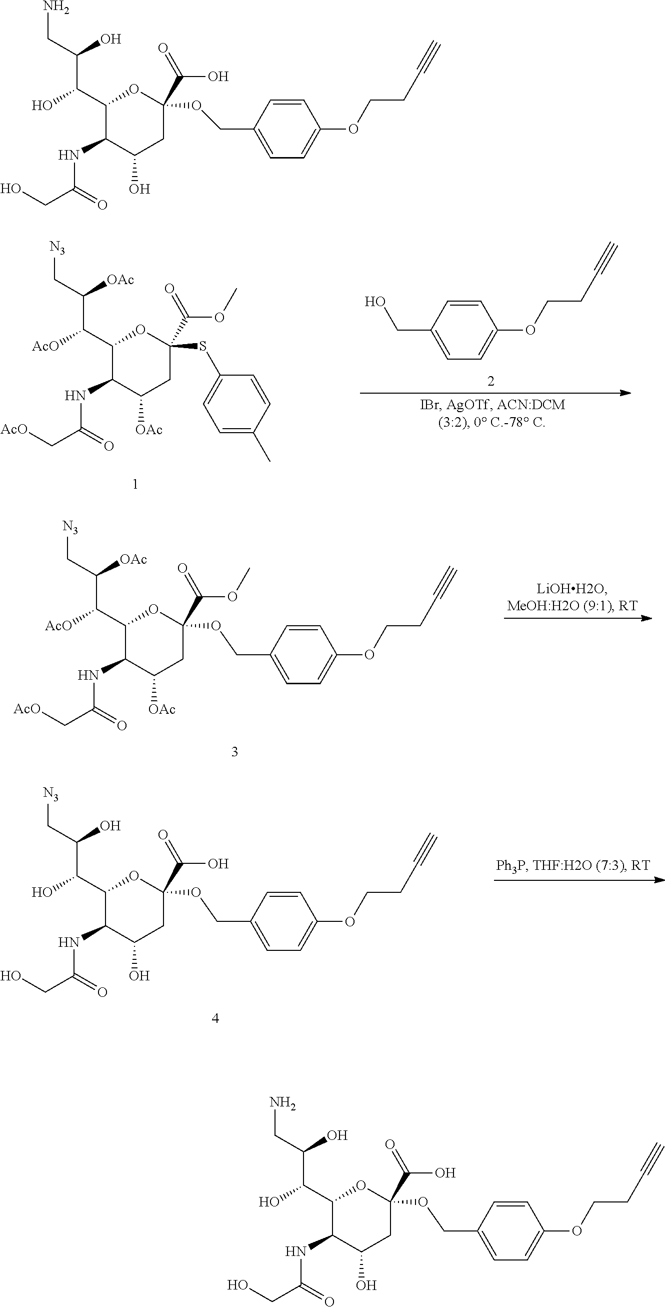

### Synthesis of (1R,2R)-1-((2R,3R,4S,6R)-4-acetoxy-3-(2-acetoxyacetamido)-6-((4-(but-3-yn-1-yloxy)benzyl)oxy)-6-(methoxycarbonyl)tetrahydro-2H-pyran-2-yl)-3-azidopropane-1,2-diyl diacetate (3)

A mixture of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-azidopropane-1,2-diyl diacetate (1, 3.0 g, 4.70 mmol) and (4-(but-3-yn-1-yloxy)phenyl)methanol (2, 4.14 g, 23.5 mmol), silver(1+) trifluoromethanesulfonate (2.41 g, 9.39 mmol) and activated 4 Å powdered molecular sieves (5.00 g) in anhydrous dichloromethane (55.0 mL) and anhydrous acetonitrile (90.0 mL) was stirred at room temperature for 1 h. The solution was cooled to −78° C. and a solution of iodobromane (1.46 g, 7.05 mmol) in dichloromethane (5.0 mL) dropwise. The reaction mixture was stirred at this temperature for 2 h then quenched with triethylamine (3.0 mL) and warmed to room temperature. The reaction mixture was filtered and washed with dichloromethane. The filtrate was washed with saturated solution of sodium bicarbonate and dried, filtered, and concentrated. The crude residue obtained was purified by silica gel column chromatography using ethyl acetate in heptane as eluents to afford the desired product (3). Yield: 2.50 g, 77%; LCMS m/z 688.70 [M−H]⁻.

### Synthesis of (2R,4S,5R,6R)-6-((1R,2R)-3-azido-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (4)

To a stirred solution of (1R,2R)-1-((2R,3R,4S,6R)-4-acetoxy-3-(2-acetoxyacetamido)-6-((4-(but-3-yn-1-yloxy)benzyl)oxy)-6-(methoxycarbonyl)tetrahydro-2H-pyran-2-yl)-3-azidopropane-1,2-diyl diacetate (3, 3.60 g, 5.21 mmol) in methanol (32.0 mL) and water (8.0 mL), was added lithium hydroxide monohydrate (1.31 g, 31.30 mmol) at 0° C. The reaction mixture was stirred at room temperature for 6 h. After completion, the reaction mixture was treated with acidic resin (Dowex 50, H⁺) to pH˜6 and the suspension was filtered. The filtrate was concentrated, and the crude residue obtained was purified by trituration with diethyl ether to afford the desired product (4). Yield: 2.0 g, 75%; LCMS m/z 507.05 [M−H]⁻.

### Synthesis of (2R,4S,5R,6R)-6-((1R,2R)-3-amino-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid Cpd. 16)

To a stirred solution of (2R,4S,5R,6R)-6-((1R,2R)-3-azido-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (4, 2.0 g, 4.92 mmol) in tetrahydrofuran (18.0 mL) and water (8.0 mL) was added triphenylphosphine (6.45 g, 24.6 mmol) at 0° C. The reaction mixture was allowed to stir at room temperature for 12 h. After completion, the reaction mixture was concentrated, and the crude was dissolved in water and ethyl acetate. The aqueous layer was separated and lyophilized to afford the crude material which was purified by prep HPLC using acetonitrile in water (+0.1% TFA) as eluents to afford the desired product (Cpd. 16). Yield: 1.30 g, 55%; LCMS m/z 483.35 [M+H]⁺; ¹H NMR (400 MHz, methanol-d₄): δ 7.29-7.25 (m, 2H), 6.91-6.87 (m, 2H), 4.75 (d, J=10.4 Hz, 1H), 4.42 (d, J=10.4 Hz, 1H), 4.13-4.05 (m, 5H), 3.91-3.85 (m, 1H), 3.81-3.71 (m, 2H), 3.68 (dd, J=13.6 Hz & 2.8 Hz, 1H), 3.34-3.31 (m, 1H), 2.99-2.94 (m, 1H), 2.84 (dd, J=12.4 Hz & 4.4 Hz, 1H), 2.64 (dt, J=6.8 Hz & 2.8 Hz, 2H), 2.32 (t, J=2.8 Hz, 1H), 1.74 (t, J=12.0 Hz, 1H).

### (2S,4S,5R,6R)-6-((1R,2R)-1,2-dihydroxy-3-(2-(4′-hydroxy-[1,1′-biphenyl]-4-yl)acetamido)propyl)-4-hydroxy-5-(2-hydroxyacetamido)-2-(oct-7-yn-1-ylthio)tetrahydro-2H-pyran-2-carboxylic acid (Compound 49)

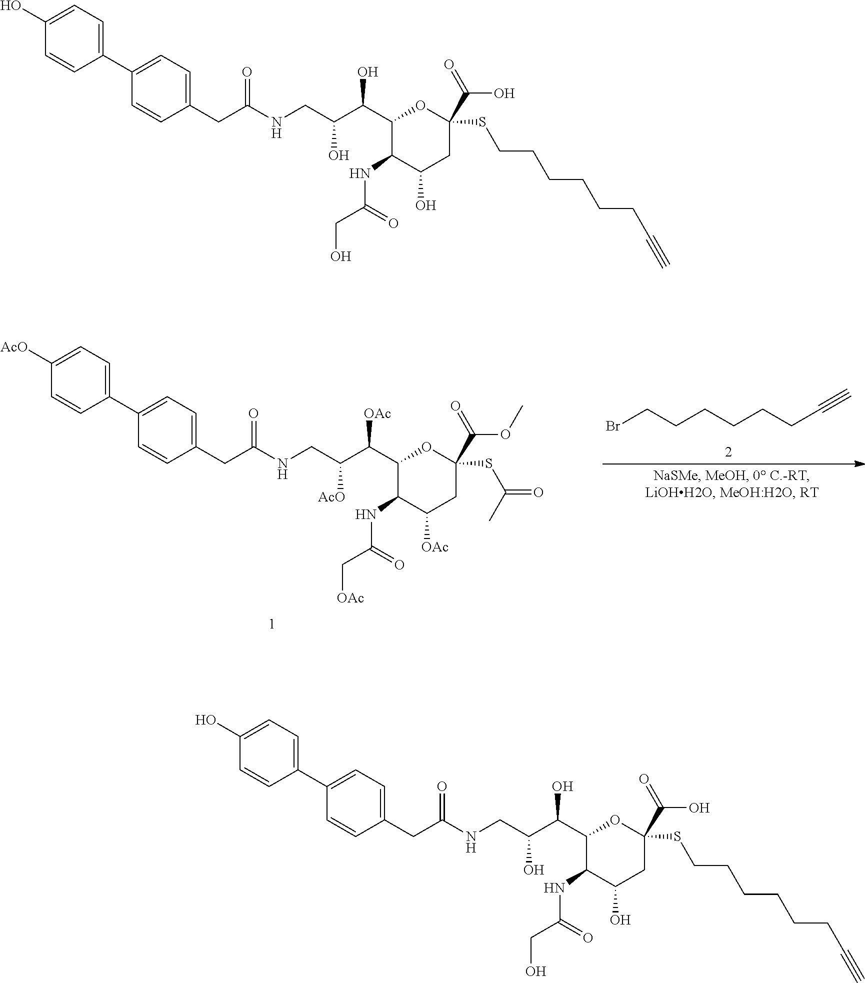

### Synthesis of (2S,4S,5R,6R)-6-((1R,2R)-1,2-dihydroxy-3-(2-(4′-hydroxy-[1,1′-biphenyl]-4-yl)acetamido)propyl)-4-hydroxy-5-(2-hydroxyacetamido)-2-(oct-7-yn-1-ylthio)tetrahydro-2H-pyran-2-carboxylic acid (Cpd. 49)

To a stirred solution of (1R,2R)-1-((2R,3R,4S,6S)-4-acetoxy-3-(2-acetoxyacetamido)-6-(acetylthio)-6-(methoxycarbonyl)tetrahydro-2H-pyran-2-yl)-3-(2-(4′-acetoxy-[1,1′-biphenyl]-4-yl)acetamido)propane-1,2-diyl diacetate (1, 0.20 g, 0.245 mmol) in methanol (3.0 mL) was added sodium thiomethoxide (0.025 g, 0.294 mmol) at 0° C. and stirred for 1 h. To the reaction mixture 8-bromooct-1-yne (2, 0.092 g, 0.490 mmol) was added at 0° C. then stirred at room temperature for 1 h. After completion, a solution of lithium hydroxide (0.061 g, 1.47 mmol) was added at room temperature and the reaction was stirred for 6 h. The mixture was neutralized by addition of acidic resin (Dowex-hydrogen form) until pH 6 and the suspension was filtered. The filtrate was concentrated, and the crude residue obtained was purified by preparative HPLC water and acetonitrile (+0.1% TFA) as eluents to afford the desired product (Cpd. 49). Yield: 0.046 g, 29%; LCMS m/z 659.60 [M+H]⁺; ¹H NMR (400 MHz, methanol-d4): δ 7.50 (d, J=8.4 Hz, 2H), 7.45-7.43 (m, 2H), 7.33 (d, J=8.4 Hz, 2H), 6.85-6.83 (m, 2H), 4.00 (s, 2H), 3.86-3.80 (m, 3H), 3.66-3.65 (m, 1H), 3.61-3.59 (m, 1H), 3.56 (s, 2H), 3.36 (dd, J=8.8 Hz & 1.6 Hz, 1H), 3.30-3.24 (m, 1H), 2.76-2.59 (m, 3H), 2.17 (t, J=2.4 Hz, 1H), 2.12 (dt, J=6.8 Hz & 2.4 Hz, 2H), 1.76 (t, J=12 Hz, 1H), 1.54-1.52 (m, 2H), 1.45-1.43 (m, 2H), 1.34-1.33 (m, 4H).

### (2R,4S,5R,6R)-2-((5-carboxypentyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Compound 121)

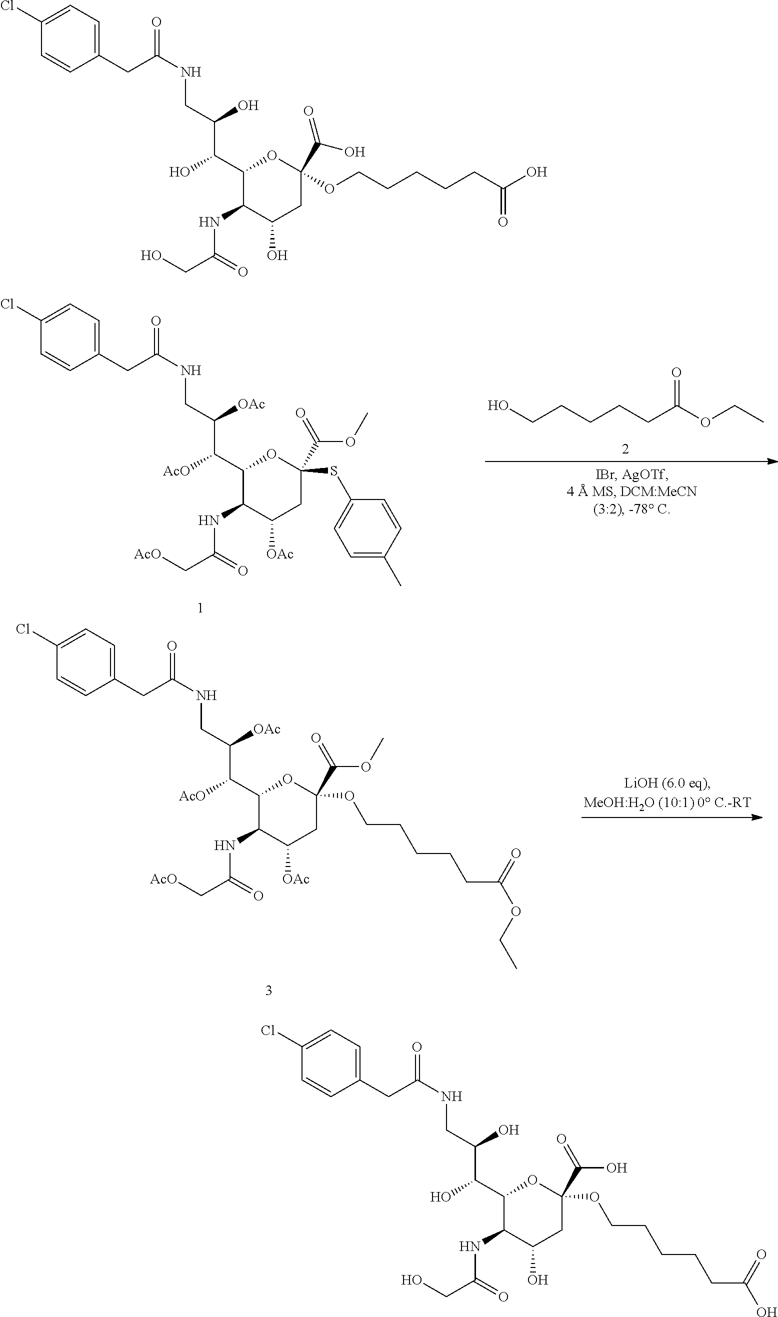 [SMILES: O=C(O)CCCCCO[C@]1(C(=O)O)C[C@H](O)[C@@H](NC(=O)CO)[C@H]([C@H](O)[C@H](O)CNC(=O)Cc2ccc(Cl)cc2)O1 ; COC(=O)[C@]1(Sc2ccc(C)cc2)C[C@H](OC(C)=O)[C@@H](NC(=O)COC(C)=O)[C@H]([C@H](OC(C)=O)[C@@H](CNC(=O)Cc2ccc(Cl)cc2)OC(C)=O)O1 ; CCOC(=O)CCCCCO ; CCOC(=O)CCCCCO[C@]1(C(=O)OC)C[C@H](OC(C)=O)[C@@H](NC(=O)COC(C)=O)[C@H]([C@H](OC(C)=O)[C@@H](CNC(=O)Cc2ccc(Cl)cc2)OC(C)=O)O1 ; O=C(O)CCCCCO[C@]1(C(=O)O)C[C@H](O)[C@@H](NC(=O)CO)[C@H]([C@H](O)[C@H](O)CNC(=O)Cc2ccc(Cl)cc2)O1]

### Synthesis of (1R,2R)-1-((2R,3R,4S,6R)-4-acetoxy-3-(2-acetoxyacetamido)-6-((6-ethoxy-6-oxohexyl)oxy)-6-(methoxycarbonyl)tetrahydro-2H-pyran-2-yl)-3-(2-(4-chlorophenyl)acetamido)propane-1,2-diyl diacetate (3)

A mixture of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (1, 0.20 g, 0.26 mmol) and ethyl 6-hydroxyhexanoate (2, 0.21 g, 1.31 mmol), silver(1+) trifluoromethanesulfonate (0.13 g, 0.53 mmol) and activated 4 Å powdered molecular sieves (1.00 g) in anhydrous dichloromethane (35 mL) and anhydrous acetonitrile (60 mL, 3.45 mol) was stirred at room temperature for 1 h. The mixture was cooled to −78° C. and a solution of iodobromane (0.08 g, 0.39 mmol) in dichloromethane (5.0 mL) was added dropwise. The reaction mixture was stirred at this temperature for 2 h then quenched with triethylamine (1.0 mL) and allowed to warm up to room temperature. The reaction mixture was filtered, and washed with dichloromethane. The filtrate was washed with saturated solution of sodium bicarbonate and dried, filtered, and concentrated. The crude product obtained was purified by silica gel column chromatography using ethyl acetate in heptane as eluents to afford the desired product (3). Yield: 0.15 g, 71%; LCMS m/z 801.90 [M+H]⁺.

### Synthesis of (2R,4S,5R,6R)-2-((5-carboxypentyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Cpd. 121)

To a stirred solution of (1R,2R)-1-((2R,3R,4S,6R)-4-acetoxy-3-(2-acetoxyacetamido)-6-((6-ethoxy-6-oxohexyl)oxy)-6-(methoxycarbonyl)tetrahydro-2H-pyran-2-yl)-3-(2-(4-chlorophenyl)acetamido)propane-1,2-diyl diacetate (3, 0.18 g, 0.23 mmol) in methanol (5 mL), was added a solution of lithium hydroxide monohydrate (0.037 g, 1.57 mmol) in water (0.5 mL). The reaction mixture was stirred at room temperature for 6 h. After completion, the reaction mixture was treated with acidic resin (Dowex 50, H⁺) to pH ˜6 and the suspension was filtered. The filtrate was concentrated, and the crude obtained was purified by prep HPLC using acetonitrile in water (+0.1% TFA) as eluents to afford the desired product (Cpd. 121). Yield: 0.032 g, 24%; LCMS m/z 591.35 [M+H]⁺; ¹H NMR (400 MHz, Methanol-d₄): δ 7.32-7.27 (m, 4H), 4.01 (s, 2H), 3.90-3.69 (m, 5H), 3.63 (dd, J=14.0 Hz & 3.2 Hz, 1H), 3.53 (s, 2H), 3.43-3.36 (m, 2H), 3.30-3.24 (m, 1H), 2.71 (dd, J=12.4 Hz & 4.0 Hz, 1H), 2.28 (t, J=7.2 Hz, 2H), 1.72 (t, J=12.0 Hz, 1H), 1.65-1.53 (m, 4H), 1.42-1.36 (m, 2H).

### (2R,4S,5R,6R)-6-((1R,2R)-3-(2-(4-chlorophenyl)acetamido)-1,2-dihydroxypropyl)-4-hydroxy-5-(2-hydroxyacetamido)-2-((10-oxo-3,6,13,16,19,22-hexaoxa-9-azapentacos-24-yn-1-yl)oxy)tetrahydro-2H-pyran-2-carboxylic acid (Compound 111)

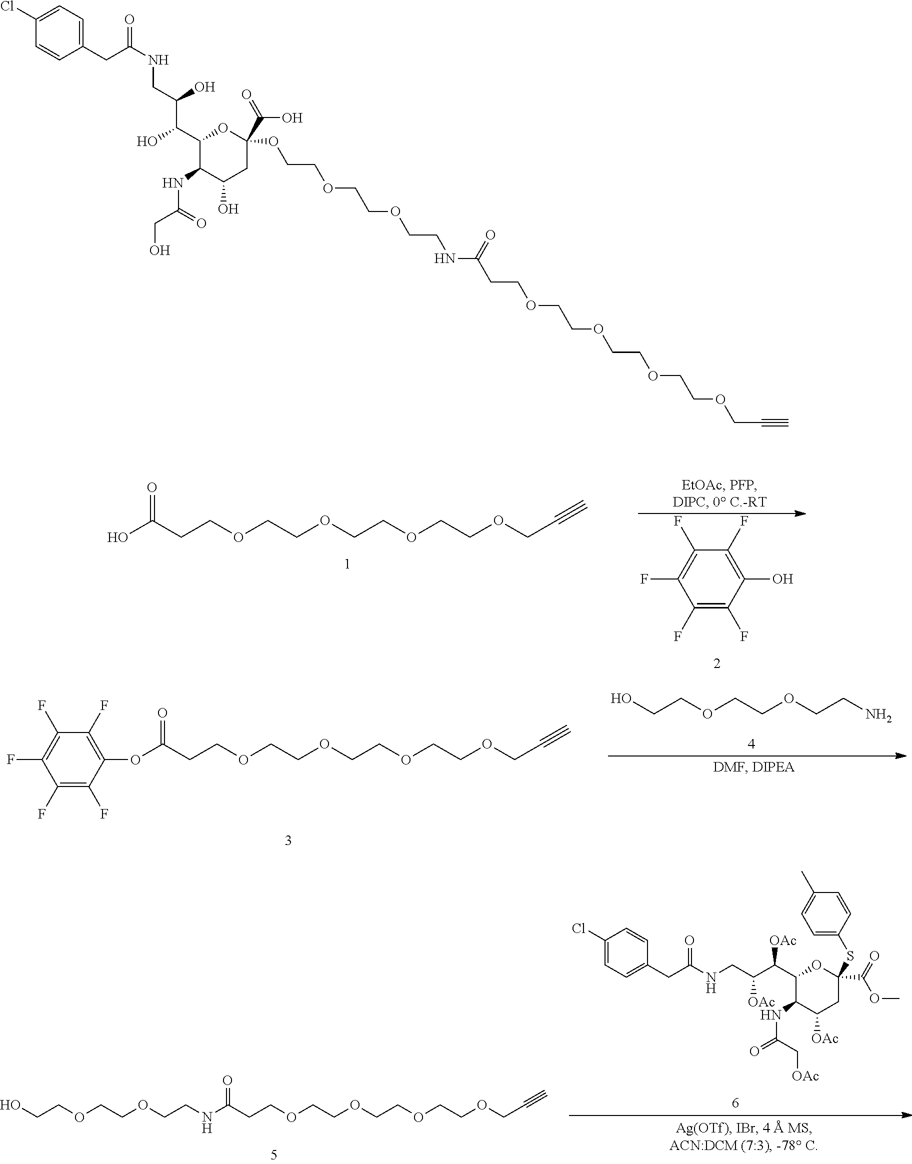

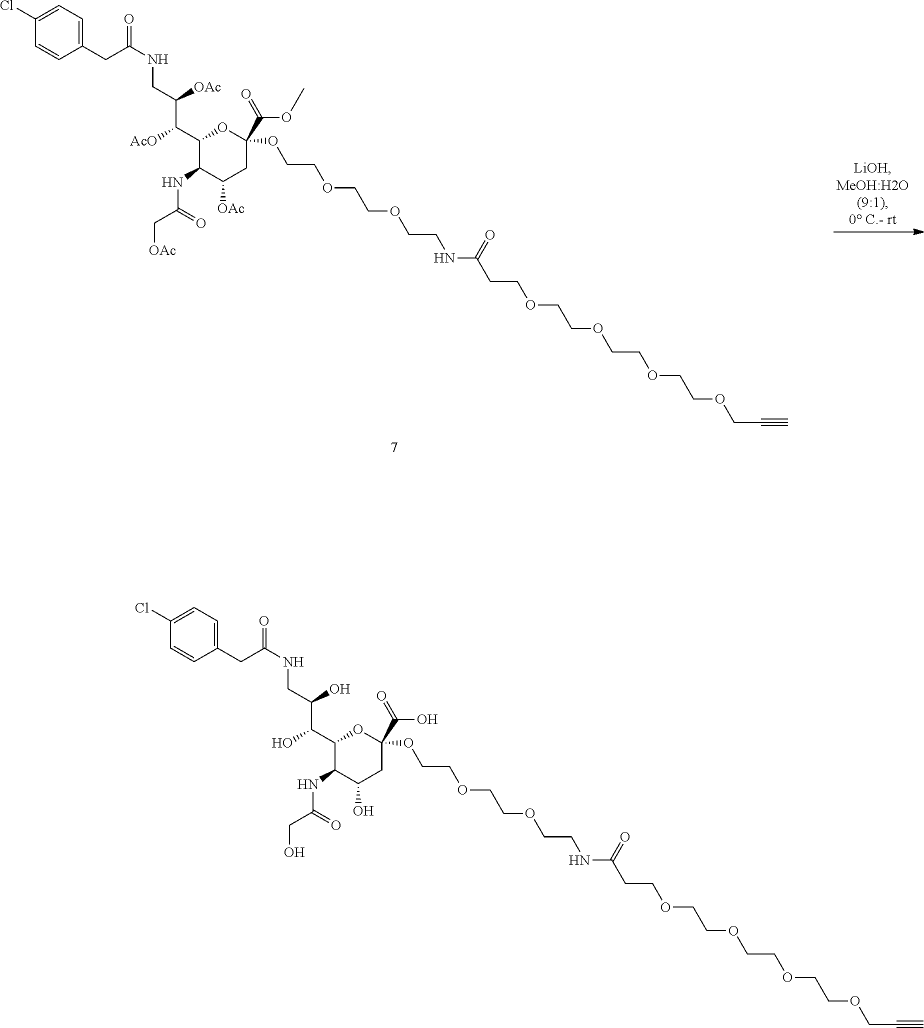 [SMILES: C#CCOCCOCCOCCOCCC(=O)NCCOCCOCCO[C@]1(C(=O)OC)C[C@H](OC(C)=O)[C@@H](NC(=O)COC(C)=O)[C@H]([C@H](OC(C)=O)[C@@H](CNC(=O)Cc2ccc(Cl)cc2)OC(C)=O)O1 ; C#CCOCCOCCOCCOCCC(=O)NCCOCCOCCO[C@]1(C(=O)O)C[C@H](O)[C@@H](NC(=O)CO)[C@H]([C@H](O)[C@H](O)CNC(=O)Cc2ccc(Cl)cc2)O1]

### Synthesis of perfluorophenyl 4,7,10,13-tetraoxahexadec-15-ynoate (3)

To a stirred solution of 4,7,10,13-tetraoxahexadec-15-ynoic acid (1, 0.50 g, 1.92 mmol) in ethyl acetate (10 mL) were added 2,3,4,5,6-pentafluorophenol (2, 0.354 g, 1.92 mmol) and N,N′-Diisopropylcarbodiimide (0.3 mL, 1.92 mmol) at 0° C. The reaction mixture was stirred at room temperature for 12 h then filtered, and the filtrate was concentrated. The crude was purified by silica gel column chromatography using ethyl acetate in hexane as eluents to afford the desired product (3). Yield: 0.58 g, 71%; ¹H NMR (400 MHz, DMSO-d₆): δ 4.12 (d, J=2.0 Hz, 2H), 3.76 (t, J=5.6 Hz, 2H), 3.55-3.50 (m, 13H), 3.02 (t, J=5.6 Hz, 2H).

### Synthesis of N-(2-(2-(2-hydroxyethoxy)ethoxy)ethyl)-4,7,10,13-tetraoxahexadec-15-ynamide (5)

To a stirred solution of 2-(2-(2-aminoethoxy)ethoxy)ethan-1-ol (4, 0.24 g, 1.64 mmol) and perfluorophenyl 4,7,10,13-tetraoxahexadec-15-ynoate (3, 0.70 g, 1.64 mmol) in dimethyl formamide (10.0 mL) was added ethylbis(propan-2-yl)amine (1.43 mL, 8.21 mmol) at 0° C. then stirred at room temperature for 12 h. After completion, the reaction mixture was concentrated, and the crude residue obtained was purified by silica gel column chromatography using ethyl acetate in hexane as eluents to afford the desired product (5). Yield: 0.40 g, 62% (over two steps); LCMS (ESI) m/z 392.25 [M+H]⁺.

### Synthesis of (1R,2R)-1-((2R,3R,4S,6R)-4-acetoxy-3-(2-acetoxyacetamido)-6-(methoxycarbonyl)-6-((10-oxo-3,6,13,16,19,22-hexaoxa-9-azapentacos-24-yn-1-yl)oxy)tetrahydro-2H-pyran-2-yl)-3-(2-(4-chlorophenyl)acetamido)propane-1,2-diyl diacetate (7)

A mixture of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (6, 0.20 g, 0.26 mmol) and N-(2-(2-(2-hydroxyethoxy)ethoxy)ethyl)-4,7,10,13-tetraoxahexadec-15-ynamide (5, 0.41 g, 1.05 mmol), silver(1+) trifluoromethanesulfonate (0.20 g, 0.78 mmol) and activated 4 Å powdered molecular sieves (1.00 g) in anhydrous dichloromethane (55 mL) and anhydrous acetonitrile (90 mL) was stirred at room temperature for 1 h. The solution was cooled to −78° C. and a solution of iodobromane (0.11 g, 0.52 mmol) in dichloromethane (5.0 mL) was added dropwise. The reaction mixture was stirred at this temperature for 2 h then quenched with triethylamine (2.0 mL) and warmed up to room temperature. The reaction mixture was filtered and washed with dichloromethane. The filtrate was washed with saturated solution of sodium bicarbonate and dried, filtered, and concentrated. The crude residue obtained was purified by silica gel column chromatography using ethyl acetate in heptane as eluents to afford the desired product (7). Yield: 0.23 g, 35%; LCMS m/z 1032.05 [M+H]⁺.

### Synthesis of (2R,4S,5R,6R)-6-((1R,2R)-3-(2-(4-chlorophenyl)acetamido)-1,2-dihydroxypropyl)-4-hydroxy-5-(2-hydroxyacetamido)-2-((10-oxo-3,6,13,16,19,22-hexaoxa-9-azapentacos-24-yn-1-yl)oxy)tetrahydro-2H-pyran-2-carboxylic acid (Cpd. 111)

To a stirred solution of (1R,2R)-1-((2R,3R,4S,6R)-4-acetoxy-3-(2-acetoxyacetamido)-6-(methoxycarbonyl)-6-((10-oxo-3,6,13,16,19,22-hexaoxa-9-azapentacos-24-yn-1-yl)oxy)tetrahydro-2H-pyran-2-yl)-3-(2-(4-chlorophenyl)acetamido)propane-1,2-diyl diacetate (7, 0.23 g, 0.22 mmol) in methanol (5 mL) was added a solution of lithium hydroxide monohydrate (0.032 g, 1.34 mmol) in water (0.5 mL). The reaction mixture was stirred at room temperature for 6 h. After completion, the reaction mixture was treated with acidic resin (Dowex 50, H⁺) to pH˜6 and the suspension was filtered. The filtrate was concentrated, and the crude residue obtained was purified by prep HPLC using acetonitrile in water (+0.1% TFA) as eluents to afford the desired product (Cpd. 111). Yield: 0.028 g, 42%; LCMS m/z 850.40 [M+H]⁺; ¹H NMR (400 MHz, Methanol-d₄): δ 7.34-7.29 (m, 4H), 4.20 (d, J=2.4 Hz, 1H), 4.04 (s, 2H), 3.92-3.82 (m, 4H), 3.76-3.61 (m, 22H), 3.58-3.55 (m, 4H), 3.41-3.37 (m, 4H), 3.32-3.25 (m, 1H), 2.87 (t, J=3.0 Hz, 1H), 2.76 (dd, J=12.4 Hz & 4.0 Hz, 1H), 2.48 (t, J=8.4 Hz, 1H), 1.76 (t, J=12.0 Hz, 1H).

### (2R,4S,5R,6R)-5-acetamido-2-((3-(but-3-yn-1-yloxy)benzyl)oxy)-6-((1R,2R)-1,2-dihydroxy-3-(3-phenoxybenzamido)propyl)-4-hydroxytetrahydro-2H-pyran-2-carboxylic acid (Compound 23)

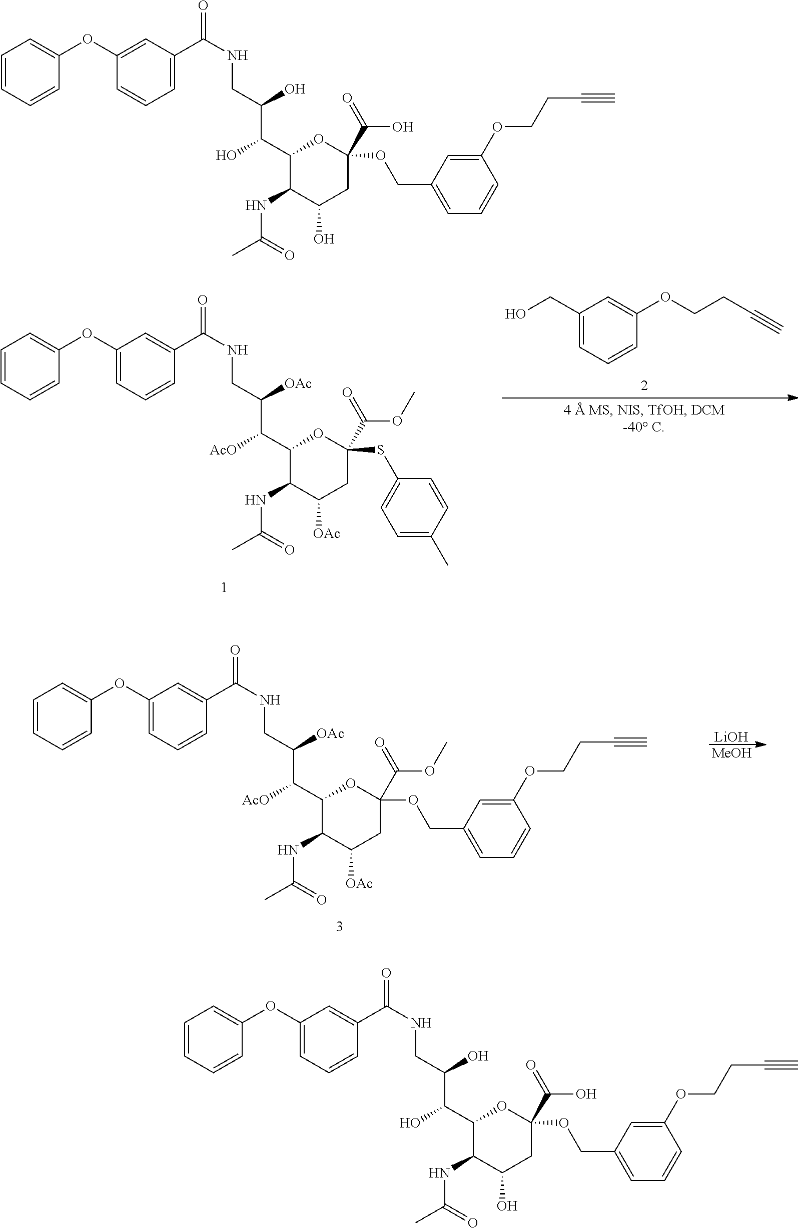

### Synthesis of (1R,2R)-1-((2R,3R,4S)-3-acetamido-4-acetoxy-6-((3-(but-3-yn-1-yloxy)benzyl)oxy)-6-(methoxycarbonyl)tetrahydro-2H-pyran-2-yl)-3-(3-phenoxybenzamido)propane-1,2-diyl diacetate (3)

A mixture of (1R,2R)-1-((2R,3R,4S,6R)-3-acetamido-4-acetoxy-6-(methoxycarbonyl)-6-(p-tolylthio)tetrahydro-2H-pyran-2-yl)-3-(3-phenoxybenzamido)propane-1,2-diyl diacetate (1, 1.00 g, 1.33 mmol), (3-(but-3-yn-1-yloxy)phenyl)methanol (2, 0.586 g, 3.33 mmol) and activated powdered 4 Å molecular sieves (1.00 g, 100% w/w) in anhydrous dichloromethane (20.0 mL) was stirred at room temperature for 15 h. The solution was then cooled to −40° C. followed by addition of 1-iodopyrrolidine-2,5-dione (0.719 g, 3.20 mmol) and trifluoromethanesulfonic acid (0.163 mL, 1.33 mmol). The reaction mixture was stirred at this temperature for another 1 h. After completion, the reaction mixture was quenched by addition of triethyl amine (neutral pH) and warmed up gradually to room temperature. The reaction mixture was filtered, and the filtrate was washed with deionized water. The organic layer was separated, dried, filtered, concentrated, and the crude residue obtained was purified by silica gel column chromatography using ethyl acetate in hexane as eluents to afford the desired product (3). Yield: 0.710 g, 66.40%; LCMS m/z 803.29 [M+H]⁺.

### Synthesis of (2R,4S,5R,6R)-5-acetamido-2-((3-(but-3-yn-1-yloxy)benzyl)oxy)-6-((1R,2R)-1,2-dihydroxy-3-(3-phenoxybenzamido)propyl)-4-hydroxytetrahydro-2H-pyran-2-carboxylic acid (Cpd. 23)

To a stirred solution of (1R,2R)-1-((2R,3R,4S)-3-acetamido-4-acetoxy-6-((3-(but-3-yn-1-yloxy)benzyl)oxy)-6-(methoxycarbonyl)tetrahydro-2H-pyran-2-yl)-3-(3-phenoxybenzamido)propane-1,2-diyl diacetate (3, 0.70 g, 0.871 mmol) in methanol:water (9:1, 10 mL), was added a solution of lithium hydroxide monohydrate (0.214 g, 5.23 mmol). The reaction mixture was stirred at room temperature for 6 h. After completion, the reaction mixture was treated with acidic resin (Dowex 50, H⁺) until pH˜6 and the suspension was filtered. The filtrate was concentrated, and the crude residue obtained was purified by prep HPLC using acetonitrile in water (+0.1% TFA) as eluents to afford the desired product (Cpd. 23). Yield: 0.097 g, 16.79%; LCMS m/z 663.45 [M+H]⁺; ¹H NMR (400 MHz, methanol-d₄) δ 7.57 (d, J=8.0 Hz, 1H), 7.47-7.35 (m, 4H), 7.20 (t, J=7.6 Hz, 1H), 7.15-7.12 (m, 2H), 7.03-7.01 (m, 2H), 6.92-6.89 (m, 2H), 6.82 (dd, J=8.0 & 2.0 Hz, 1H), 4.81 (d, J=11.6 Hz, 1H), 4.51 (d, J=11.6 Hz, 1H), 4.06 (t, J=6.8, 2H), 4.04-4.00 (m, 1H), 3.85-3.74 (m, 4H), 3.55-3.53 (m, 1H), 3.48-3.46 (m, 1H), 2.77 (dd, J=12.8 & 4.4 Hz, 1H), 2.62 (td, J=6.8 & 2.8 Hz, 2H), 2.31 (t, J=2.8 Hz, 1H), 1.98 (s, 3H), 1.81 (t, J=12.4 Hz, 1H).

### (2R,4S,5R,6R)-6-((1R,2R)-3-(2-([1,1′-biphenyl]-4-yl)acetamido)-1,2-dihydroxypropyl)-2-((3-(but-3-yn-1-yloxy)benzyl)oxy)-4-hydroxy-5-(2-hydroxyacetamido)tetrahydro-2H-pyran-2-carboxylic acid (Compound 38)

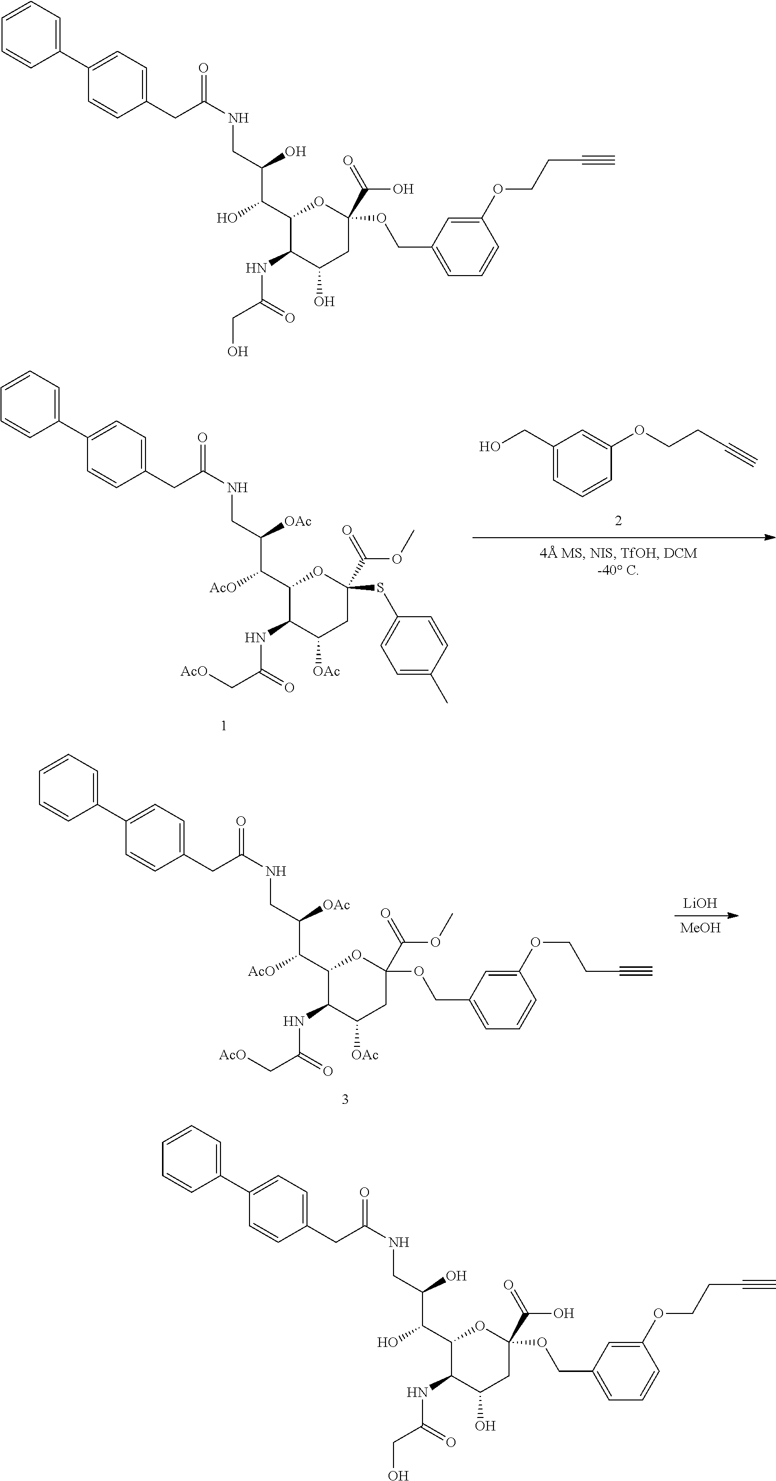

### Synthesis of (1R,2R)-3-(2-([1,1′-biphenyl]-4-yl)acetamido)-1-((2R,3R,4S)-4-acetoxy-3-(2-acetoxyacetamido)-6-((3-(but-3-yn-1-yloxy)benzyl)oxy)-6-(methoxycarbonyl)tetrahydro-2H-pyran-2-yl)propane-1,2-diyl diacetate (3)

A mixture of (1R,2R)-3-(2-([1,1′-biphenyl]-4-yl)acetamido)-1-((2R,3R,4S,6R)-4-acetoxy-3-(2-acetoxyacetamido)-6-(methoxycarbonyl)-6-(p-tolylthio)tetrahydro-2H-pyran-2-yl)propane-1,2-diyl diacetate (1, 1.00 g, 1.24 mmol), (3-(but-3-yn-1-yloxy)phenyl)methanol (2, 0.546 g, 3.10 mmol) and activated powdered 4 Å molecular sieves (1.00 g, 100% w/w) in anhydrous dichloromethane (20.0 mL) was stirred at room temperature for 15 h. The solution was then cooled to −40° C. followed by addition of 1-iodopyrrolidine-2,5-dione (0.669 g, 2.97 mmol) and trifluoromethanesulfonic acid (0.153 mL, 1.24 mmol). The reaction mixture was stirred at this temperature for another 1 h. After completion, the reaction mixture was quenched by addition of triethyl amine (neutral pH) and warmed up gradually to room temperature. The reaction mixture was filtered, and the filtrate was washed with deionized water. The organic layer was separated, dried, filtered, concentrated, and the crude residue obtained was purified by silica gel column chromatography using ethyl acetate in hexane as eluents to afford the desired product (3). Yield: 0.750 g, 70.46%; LCMS m/z 859.32 [M+H]⁺.

### Synthesis of (2R,4S,5R,6R)-6-((1R,2R)-3-(2-([1,1′-biphenyl]-4-yl)acetamido)-1,2-dihydroxypropyl)-2-((3-(but-3-yn-1-yloxy)benzyl)oxy)-4-hydroxy-5-(2-hydroxyacetamido)tetrahydro-2H-pyran-2-carboxylic acid (Cpd. 38)

To a stirred solution of (1R,2R)-3-(2-([1,1′-biphenyl]-4-yl)acetamido)-1-((2R,3R,4S)-4-acetoxy-3-(2-acetoxyacetamido)-6-((3-(but-3-yn-1-yloxy)benzyl)oxy)-6-(methoxycarbonyl)tetrahydro-2H-pyran-2-yl)propane-1,2-diyl diacetate (3, 0.75 g, 0.873 mmol) in methanol (10 mL), was added a solution of lithium hydroxide monohydrate (0.214 g, 5.24 mmol) in water (0.4 mL). The reaction mixture was stirred at room temperature for 6 h then treated with acidic resin (Dowex 50, H⁺) until pH ˜6 and the suspension was filtered. The filtrate was concentrated, and the crude residue obtained was purified by prep HPLC using acetonitrile in water (+0.1% TFA) as eluents to afford the desired product (Cpd. 38). Yield: 0.117 g, 19.80%; LCMS m/z 677.35 [M+H]⁺; ¹H NMR (400 MHz, methanol-d₄) δ 7.56-7.52 (m, 4H), 7.41-7.28 (m, 5H), 7.19 (t, J=8.0 Hz, 1H), 6.91-6.87 (m, 2H), 6.81 (dd, J=8.4, 2.4 Hz, 1H), 4.78 (d, J=11.6 Hz, 1H), 4.47 (d, J=11.6 Hz, 1H), 4.07-4.02 (m, 4H), 3.95-3.78 (m, 4H), 3.69 (dd, J=14.0 & 3.2 Hz, 1H), 3.58 (bs, 2H), 3.41 (dd, J=8.4 & 1.2 Hz, 1H), 3.30-3.24 (m, 1H), 2.80-2.76 (m, 1H), 2.62 (td, J=6.4 & 2.4 Hz, 2H), 2.31 (t, J=2.4 Hz, 1H), 1.81 (t, J=12.0 Hz, 1H).

### (2R,4S,5R,6R)-5-acetamido-2-((4-(but-3-yn-1-yloxy)benzyl)oxy)-6-((1R,2R)-1,2-dihydroxy-3-(3-phenoxybenzamido)propyl)-4-hydroxytetrahydro-2H-pyran-2-carboxylic acid (Compound 22)

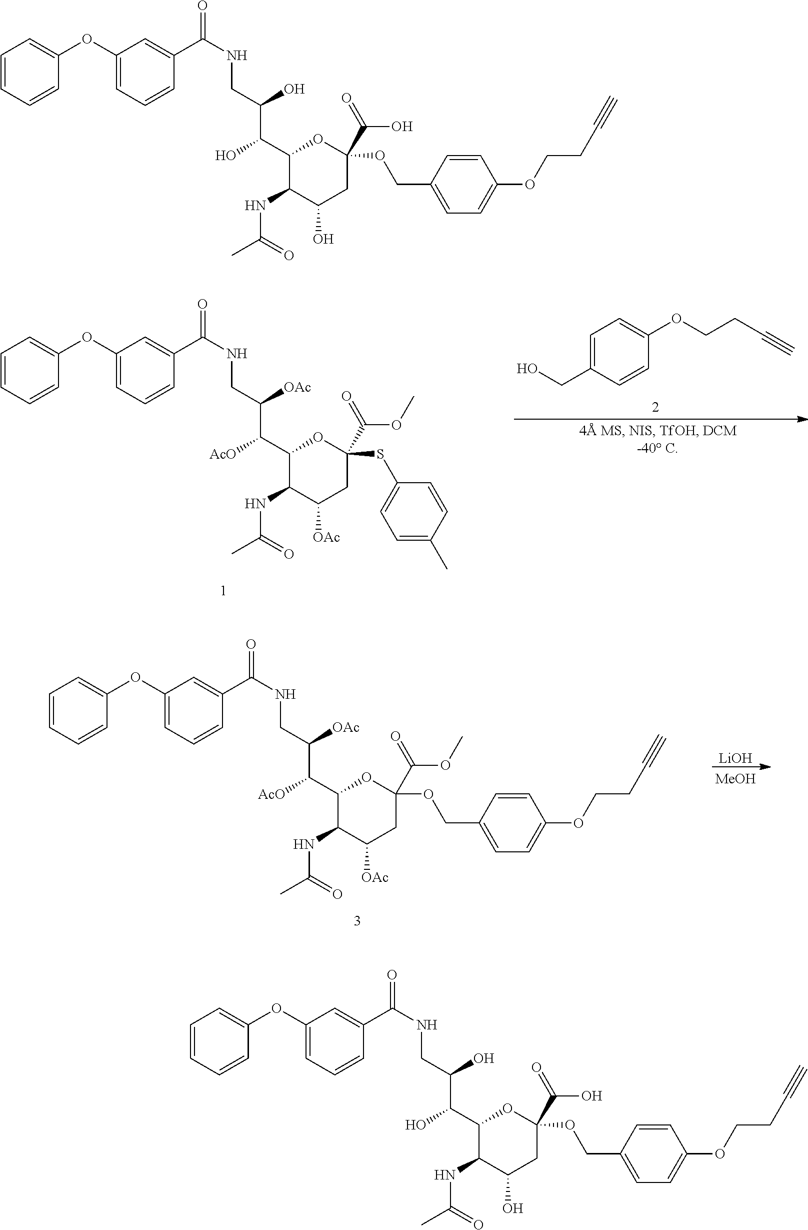

### Synthesis of (1R,2R)-1-((2R,3R,4S)-3-acetamido-4-acetoxy-6-((4-(but-3-yn-1-yloxy)benzyl)oxy)-6-(methoxycarbonyl)tetrahydro-2H-pyran-2-yl)-3-(3-phenoxybenzamido)propane-1,2-diyl diacetate (3)

A mixture of (1R,2R)-1-((2R,3R,4S,6R)-3-acetamido-4-acetoxy-6-(methoxycarbonyl)-6-(p-tolylthio)tetrahydro-2H-pyran-2-yl)-3-(3-phenoxybenzamido)propane-1,2-diyl diacetate (1, 1.00 g, 1.33 mmol), (4-(but-3-yn-1-yloxy)phenyl)methanol (2, 0.575 g, 3.27 mmol) and activated powdered 4 Å molecular sieves (1.00 g, 100% w/w) in anhydrous dichloromethane (20.0 mL) was stirred at room temperature for 15 h. The solution was then cooled to −40° C. followed by addition of 1-iodopyrrolidine-2,5-dione (0.705 g, 3.14 mmol) and trifluoromethanesulfonic acid (0.163 mL, 1.33 mmol). The reaction mixture was stirred at this temperature for another 1 h. After completion, the reaction mixture was quenched by addition of triethyl amine (neutral pH) and warmed up gradually to room temperature. The reaction mixture was filtered, and the filtrate was washed with deionized water. The organic layer was separated, dried, filtered, concentrated, and the crude residue obtained was purified by silica gel column chromatography using ethyl acetate in hexane as eluents to afford the desired product (3). Yield: 0.740 g, 69.21%; LCMS m/z 803.36 [M+H]⁺.

### Synthesis of (2R,4S,5R,6R)-5-acetamido-2-((4-(but-3-yn-1-yloxy)benzyl)oxy)-6-((1R,2R)-1,2-dihydroxy-3-(3-phenoxybenzamido)propyl)-4-hydroxytetrahydro-2H-pyran-2-carboxylic acid (Cpd. 22)

To a stirred solution of (1R,2R)-1-((2R,3R,4S)-3-acetamido-4-acetoxy-6-((4-(but-3-yn-1-yloxy)benzyl)oxy)-6-(methoxycarbonyl)tetrahydro-2H-pyran-2-yl)-3-(3-phenoxybenzamido)propane-1,2-diyl diacetate (3, 0.74 g, 0.921 mmol) in methanol:water (9:1, 10 mL), was added a solution of lithium hydroxide monohydrate (0.226 g, 5.53 mmol). The reaction mixture was stirred at room temperature for 6 h. After completion, the reaction mixture was treated with acidic resin (Dowex 50, H⁺) until pH˜6 and the suspension was filtered. The filtrate was concentrated, and the crude residue obtained was purified by prep HPLC using acetonitrile in water (+0.1% TFA) as eluents to afford the desired product (Cpd. 22). Yield: 0.170 g, 27.83%; LCMS m/z 663.30 [M+H]⁺; ¹H NMR (400 MHz, methanol-d₄) δ 7.57 (d, J=8.0 Hz, 1H), 7.47-7.34 (m, 4H), 7.23 (d, J=8.8 Hz, 2H), 7.15-7.11 (m, 2H), 7.03-6.98 (m, 2H), 4.75 (d, J=10.8 Hz, 1H), 4.42 (d, J=10.8 Hz, 1H), 4.06-4.01 (m, 3H), 3.83-3.69 (m, 4H), 3.59-3.45 (m, 2H), 2.73 (dd, J=12.8 & 4.4 Hz, 1H), 2.62 (td, J=6.8 & 2.8 Hz, 2H), 2.31 (t, J=2.8 Hz, 1H), 1.98 (s, 3H), 1.77 (t, J=12.8 Hz, 1H).

### (2R,4S,5R,6R)-6-((1R,2R)-3-(2-([1,1′-biphenyl]-4-yl)acetamido)-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (Compound 37)

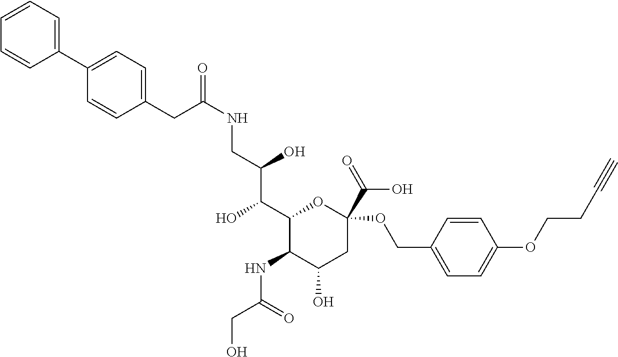 [SMILES: C#CCCOc1ccc(CO[C@]2(C(=O)O)C[C@H](O)[C@@H](NC(=O)CO)[C@H]([C@H](O)[C@H](O)CNC(=O)Cc3ccc(-c4ccccc4)cc3)O2)cc1]

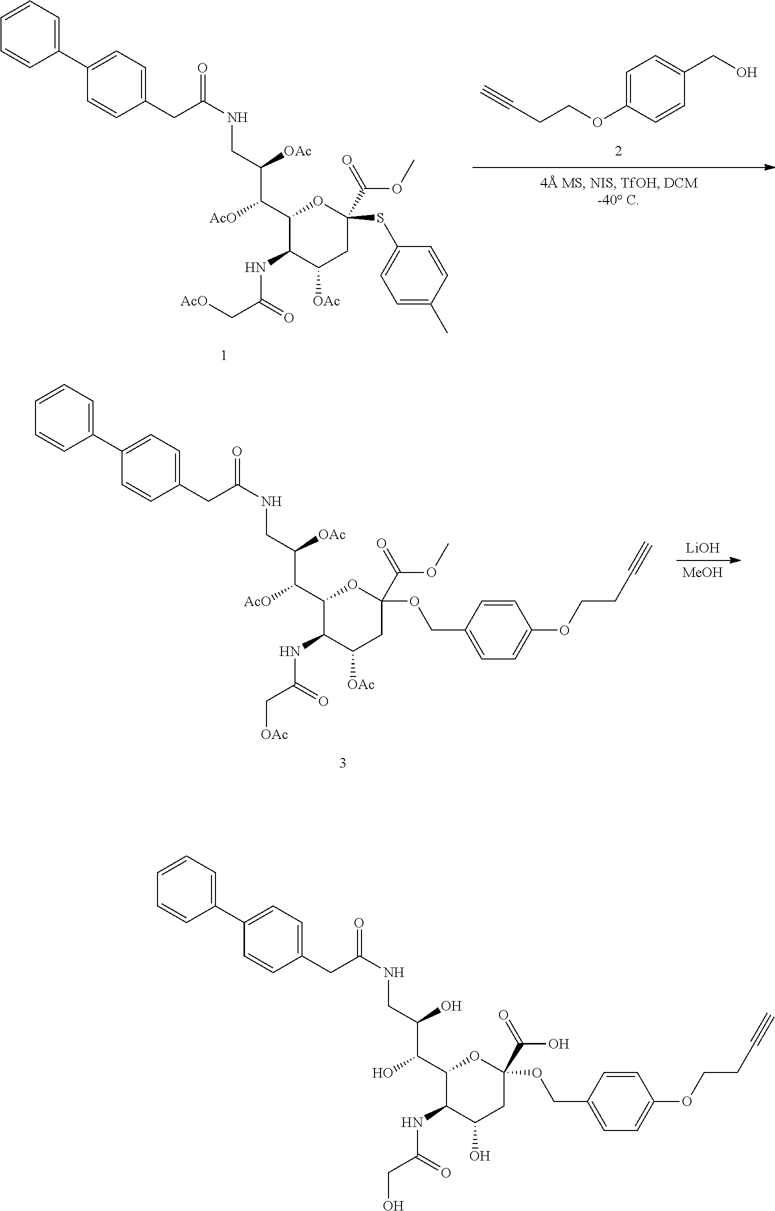

### Synthesis of (1R,2R)-3-(2-([1,1′-biphenyl]-4-yl)acetamido)-1-((2R,3R,4S)-4-acetoxy-3-(2-acetoxyacetamido)-6-((4-(but-3-yn-1-yloxy)benzyl)oxy)-6-(methoxycarbonyl)tetrahydro-2H-pyran-2-yl)propane-1,2-diyl diacetate (3)

A mixture of (1R,2R)-3-(2-([1,1′-biphenyl]-4-yl)acetamido)-1-((2R,3R,4S,6R)-4-acetoxy-3-(2-acetoxyacetamido)-6-(methoxycarbonyl)-6-(p-tolylthio)tetrahydro-2H-pyran-2-yl)propane-1,2-diyl diacetate (1, 1.00 g, 1.24 mmol), (4-(but-3-yn-1-yloxy)phenyl)methanol (2, 0.546 g, 3.10 mmol) and activated powdered 4 Å molecular sieves (1.00 g, 100% w/w) in anhydrous dichloromethane (20.0 mL) was stirred at room temperature for 15 h. The solution was then cooled to −40° C. followed by addition of 1-iodopyrrolidine-2,5-dione (0.669 g, 2.97 mmol) and trifluoromethanesulfonic acid (0.152 mL, 1.24 mmol). The reaction mixture was stirred at this temperature for another 1 h. After completion, the reaction mixture was quenched by addition of triethyl amine (pH 6) and warmed up gradually to room temperature. The reaction mixture was filtered, and the filtrate was washed with deionized water. The organic layer was separated, dried, filtered, concentrated, and the crude residue obtained was purified by silica gel column chromatography using ethyl acetate in hexane as eluents to afford the desired product (3). Yield: 0.800 g, 75.16%; LCMS m/z 859.35 [M+H]⁺.

### Synthesis of (2R,4S,5R,6R)-6-((1R,2R)-3-(2-([1,1′-biphenyl]-4-yl)acetamido)-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (Cpd. 37)

To a stirred solution of (1R,2R)-3-(2-([1,1′-biphenyl]-4-yl)acetamido)-1-((2R,3R,4S)-4-acetoxy-3-(2-acetoxyacetamido)-6-((3-(but-3-yn-1-yloxy)benzyl)oxy)-6-(methoxycarbonyl)tetrahydro-2H-pyran-2-yl)propane-1,2-diyl diacetate (3, 0.800 g, 0.931 mmol) in methanol (10 mL), was added a solution of lithium hydroxide monohydrate (0.133 g, 5.99 mmol) in water (0.4 mL). The reaction mixture was stirred at room temperature for 6 h. After completion, the reaction mixture was treated with acidic resin (Dowex 50, H⁺) until pH ˜6 and the suspension was filtered. The filtrate was concentrated, and the crude residue obtained was purified by prep HPLC using to afford the desired product water and acetonitrile (+0.1% TFA) as eluents to afford the desired product (Cpd. 37). Yield: 0.173 g, 27.45%; LCMS m/z 677.25 [M+H]⁺; ¹H NMR (400 MHz, methanol-d₄) δ 7.58-7.51 (m, 4H), 7.41-7.29 (m, 5H), 7.23-7.20 (m, 2H), 6.86-6.83 (m, 2H), 4.72 (d, J=10.4 Hz, 1H), 4.39 (d, J=10.3 Hz, 1H), 4.05-4.02 (m, 4H), 3.96-3.89 (m, 1H), 3.86-3.77 (m, 3H), 3.73-3.69 (m, 1H), 3.58 (bs, 1H), 3.41 (dd, J=8.8 & 1.6 Hz, 1H 2H), 3.30-3.23 (m, 1H), 3.59 (bs, 2H), 3.45 (d, J=9.6 Hz, 1H), 2.77-2.73 (m, 1H), 2.61 (td, J=6.8 & 2.8 Hz, 2H), 2.32 (t, J=2.8 Hz, 1H), 1.78 (t, J=12.4 Hz, 1H).

### (2R,4S,5R,6R)-5-acetamido-6-((1R,2R)-1,2-dihydroxy-3-((3-phenoxybenzyl)amino)propyl)-4-hydroxy-2-(2-(2-(prop-2-yn-1-yloxy)ethoxy)ethoxy)tetrahydro-2H-pyran-2-carboxylic acid (Compound 17)

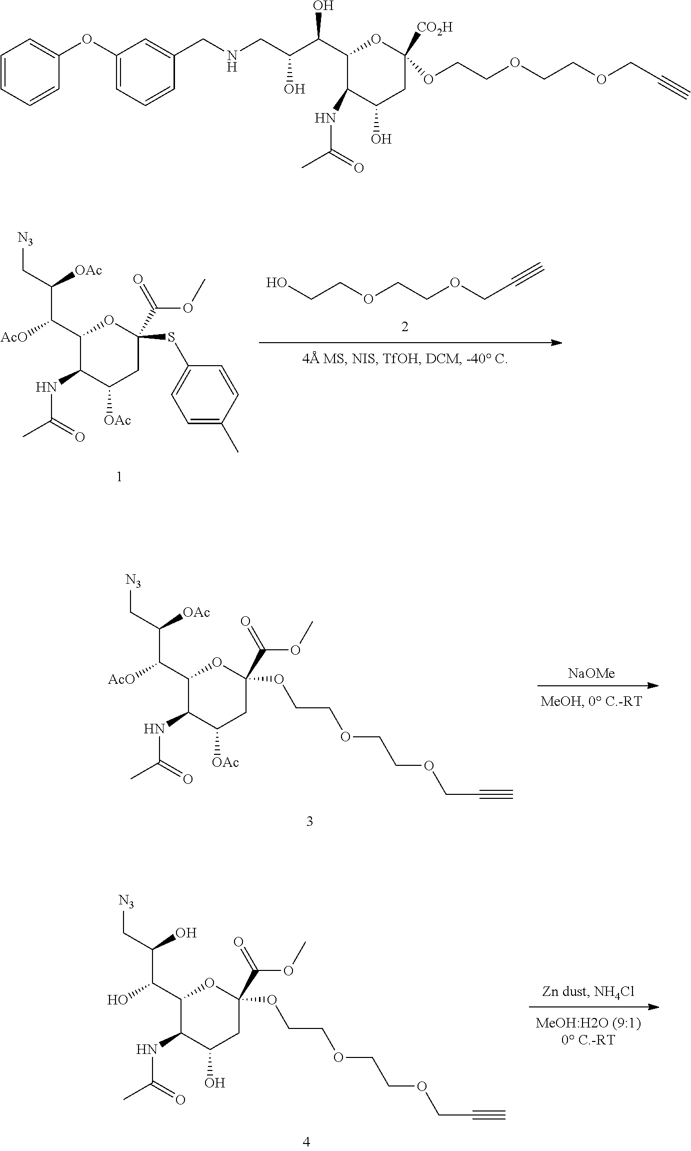

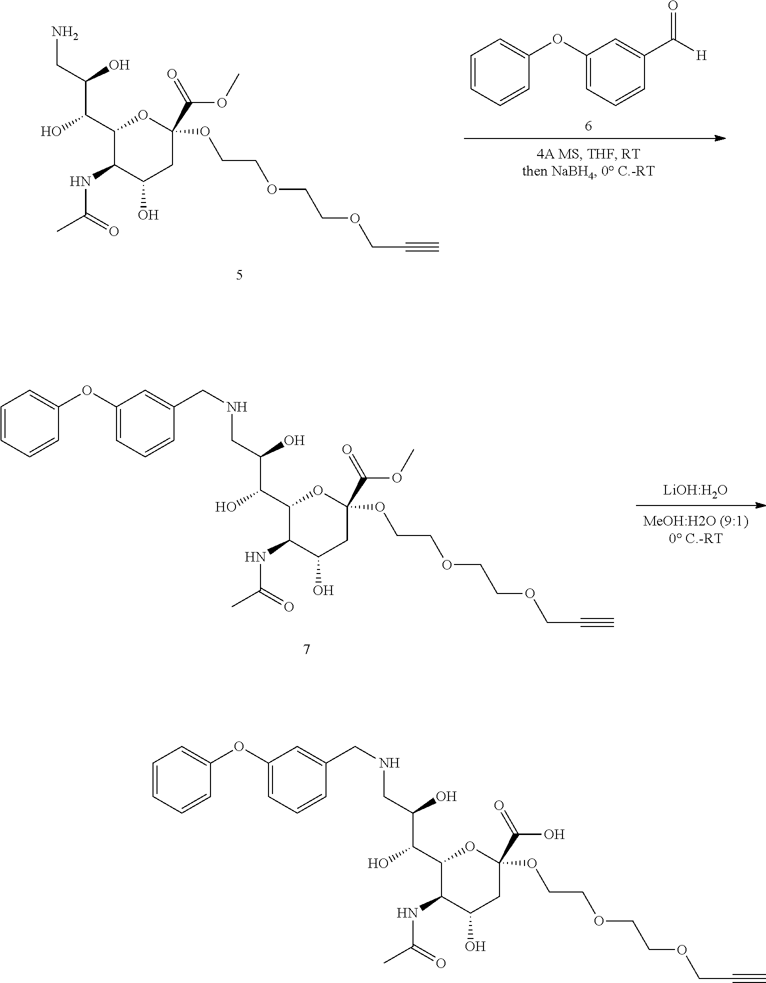

### Synthesis of (1R,2R)-1-((2R,3R,4S,6R)-3-acetamido-4-acetoxy-6-(methoxycarbonyl)-6-(2-(2-(prop-2-yn-1-yloxy)ethoxy)ethoxy)tetrahydro-2H-pyran-2-yl)-3-azidopropane-1,2-diyl diacetate (3)

In an argon atmosphere, to a solution of (1R,2R)-1-((2R,3R,4S,6R)-3-acetamido-4-acetoxy-6-(methoxycarbonyl)-6-(p-tolylthio)tetrahydro-2H-pyran-2-yl)-3-azidopropane-1,2-diyl diacetate (1, 2.0 g, 3.44 mmol) and 2-(2-(prop-2-yn-1-yloxy)ethoxy)ethan-1-ol (1.24 g, 8.61 mmol) was added activated powdered 4 Å molecular sieves (2.00 g, 100% w/w) in anhydrous dichloromethane (30.0 mL) and the mixture was stirred at room temperature for 15 h. The solution was then cooled to −40° C. followed by addition of 1-iodopyrrolidine-2,5-dione (1.94 g, 8.61 mmol) and trifluoromethanesulfonic acid (0.304 mL, 3.44 mmol). The reaction mixture was stirred at the same temperature for another 2 h. After completion, the reaction mixture was quenched by addition of triethyl amine (neutral pH) and warmed up gradually to room temperature. The reaction mixture was filtered and the filtrate was washed with water. The organic layer was separated, dried, filtered and concentrated. The crude residue obtained was purified by silica gel column chromatography using ethyl acetate in hexane to afford the desired product (1R,2R)-1-((2R,3R,4S,6R)-3-acetamido-4-acetoxy-6-(methoxycarbonyl)-6-(2-(2-(prop-2-yn-1-yloxy)ethoxy)ethoxy)tetrahydro-2H-pyran-2-yl)-3-azidopropane-1,2-diyl diacetate (3) as a mixture of anomers. Yield: 1.94 g, 93.77%; ELSD-MS m/z 618.10 [M+H3O]⁺.

### Synthesis methyl (2R,4S,5R,6R)-5-acetamido-6-((1R,2R)-3-azido-1,2-dihydroxypropyl)-4-hydroxy-2-(2-(2-(prop-2-yn-1-yloxy)ethoxy)ethoxy)tetrahydro-2H-pyran-2-carboxylate (4)

To a cooled solution (0° C.) of (1R,2R)-1-((2R,3R,4S,6R)-3-acetamido-4-acetoxy-6-(methoxycarbonyl)-6-(2-(2-(prop-2-yn-1-yloxy)ethoxy)ethoxy)tetrahydro-2H-pyran-2-yl)-3-azidopropane-1,2-diyl diacetate (1, 1.85 g; 3.08 mmol) in methanol (20.0 mL) was added 25% sodium methoxide in methanol (0.070 mL, 0.308 mmol, pH 9-10). The reaction mixture was stirred for 1 h at room temperature. The solution was neutralized with Dowex-Hydrogen form until neutral pH, filtered and the filtrate was concentrated to afford the desired crude product methyl (2R,4S,5R,6R)-5-acetamido-6-((1R,2R)-3-azido-1,2-dihydroxypropyl)-4-hydroxy-2-(2-(2-(prop-2-yn-1-yloxy)ethoxy)ethoxy)tetrahydro-2H-pyran-2-carboxylate (4) which was used for next step without further purification. Yield: 1.40 g, Crude; ELSD-MS m/z 475.05 [M+H]⁺.

### Synthesis of methyl (2R,4S,5R,6R)-5-acetamido-6-((1R,2R)-3-amino-1,2-dihydroxypropyl)-4-hydroxy-2-(2-(2-(prop-2-yn-1-yloxy)ethoxy)ethoxy)tetrahydro-2H-pyran-2-carboxylate (5)

To a solution of methyl (2R,4S,5R,6R)-5-acetamido-6-((1R,2R)-3-azido-1,2-dihydroxypropyl)-4-hydroxy-2-(2-(2-(prop-2-yn-1-yloxy)ethoxy)ethoxy)tetrahydro-2H-pyran-2-carboxylate (4, 1.40 g, 2.95 mmol) in methanol:water (9:1, 20.0 mL), zinc dust (1.93 g, 29.51 mmol) and ammonium chloride (1.58 g, 29.51 mmol) were added at 0° C. and the reaction mixture was stirred at room temperature for 1 h. After completion, the reaction mixture was filtered through celite, washed with methanol and the filtrate was concentrated to afford the desired crude product methyl (2R,4S,5R,6R)-5-acetamido-6-((1R,2R)-3-amino-1,2-dihydroxypropyl)-4-hydroxy-2-(2-(2-(prop-2-yn-1-yloxy)ethoxy)ethoxy)tetrahydro-2H-pyran-2-carboxylate (5). The crude residue was used for the next reaction without further purification. Yield: 1.15 g, 86.90% Crude; ELSD-MS m/z 449.05 [M+H]⁺.

### Synthesis of methyl (2R,4S,5R,6R)-5-acetamido-6-((1R,2R)-1,2-dihydroxy-3-((3-phenoxybenzyl)amino)propyl)-4-hydroxy-2-(2-(2-(prop-2-yn-1-yloxy)ethoxy)ethoxy)tetrahydro-2H-pyran-2-carboxylate (7)

To a solution of methyl (2R,4S,5R,6R)-5-acetamido-6-((1R,2R)-3-amino-1,2-dihydroxypropyl)-4-hydroxy-2-(2-(2-(prop-2-yn-1-yloxy)ethoxy)ethoxy)tetrahydro-2H-pyran-2-carboxylate (5, 1.10 g, 2.45 mmol) and 3-phenoxybenzaldehyde (0.534 g, 2.70 mmol) in methanol (10.0 mL) was added activated 4 Å MS (1.1 g, 100% w/w) and stirred for 12 h at room temperature. The reaction mixture was then cooled to 0° C. followed by addition of sodium borohydride (0.93 g, 2.45 mmol) and the reaction mixture was stirred at room temperature for 12 h. After completion, the reaction mixture was quenched by addition of aqueous ammonium chloride (neutral pH), filtered through celite, washed with methanol and the filtrate was concentrated to afford the desired crude product methyl (2R,4S,5R,6R)-5-acetamido-6-((1R,2R)-1,2-dihydroxy-3-((3-phenoxybenzyl)amino)propyl)-4-hydroxy-2-(2-(2-(prop-2-yn-1-yloxy)ethoxy)ethoxy)tetrahydro-2H-pyran-2-carboxylate (7). The crude residue was used for the next reaction without further purification. Yield: 1.10 g, 71.11% Crude; ELSD-MS m/z 631.05 [M+H]⁺.

### Synthesis of (2R,4S,5R,6R)-5-acetamido-6-((1R,2R)-1,2-dihydroxy-3-((3-phenoxybenzyl)amino)propyl)-4-hydroxy-2-(2-(2-(prop-2-yn-1-yloxy)ethoxy)ethoxy)tetrahydro-2H-pyran-2-carboxylic acid (Cpd. 17)

To a stirred solution of methyl (2R,4S,5R,6R)-5-acetamido-6-((1R,2R)-1,2-dihydroxy-3-((3-phenoxybenzyl)amino)propyl)-4-hydroxy-2-(2-(2-(prop-2-yn-1-yloxy)ethoxy)ethoxy)tetrahydro-2H-pyran-2-carboxylate (7, 0.90 g, 1.43 mmol) in methanol:water (9:1, 10 mL), was added a solution of lithium hydroxide monohydrate (0.119 g, 2.85 mmol). The reaction mixture was stirred at room temperature for 4 h. After completion, the reaction mixture was treated with acidic resin (Dowex 50, H⁺) up to pH ˜6 and the suspension was filtered. The filtrate was concentrated. The crude material was purified by prep HPLC using acetonitrile in water (+0.1% TFA) as eluents to afford the desired product (2R,4S,5R,6R)-5-acetamido-6-((1R,2R)-1,2-dihydroxy-3-((3-phenoxybenzyl)amino)propyl)-4-hydroxy-2-(2-(2-(prop-2-yn-1-yloxy)ethoxy)ethoxy)tetrahydro-2H-pyran-2-carboxylic acid (Cpd. 17). Yield: 0.022 g, 2.50%; LCMS m/z 617.30 [M+H]+; ¹H NMR (400 MHz, methanol-d₄) δ 7.46-7.37 (m, 3H), 7.22 (d, J=7.6 Hz, 1H), 7.17-7.14 (m, 2H), 7.07-7.02 (m, 3H), 4.23 (s, 2H), 4.16 (d, J=2.4 Hz, 2H), 4.10 (td, J=8.8 & 3.2 Hz, 1H), 3.89-3.82 (m, 1H), 3.74-3.58 (m, 9H), 3.53-3.51 (m, 1H), 3.42-3.35 (m, 2H), 3.07-3.01 (m, 1H), 2.83 (t, J=2.8 Hz, 1H), 2.79-2.75 (m, 1H), 2.00 (s, 3H), 1.68 (t, J=12.0 Hz, 1H).

### (2R,4S,5R,6R)-2-(2-(2-(but-3-yn-1-yloxy)ethoxy)ethoxy)-6-((1R,2R)-1,2-dihydroxy-3-(2-(4′-hydroxy-[1,1′-biphenyl]-4-yl)acetamido)propyl)-4-hydroxy-5-(2-hydroxyacetamido)tetrahydro-2H-pyran-2-carboxylic acid (Compound 83)

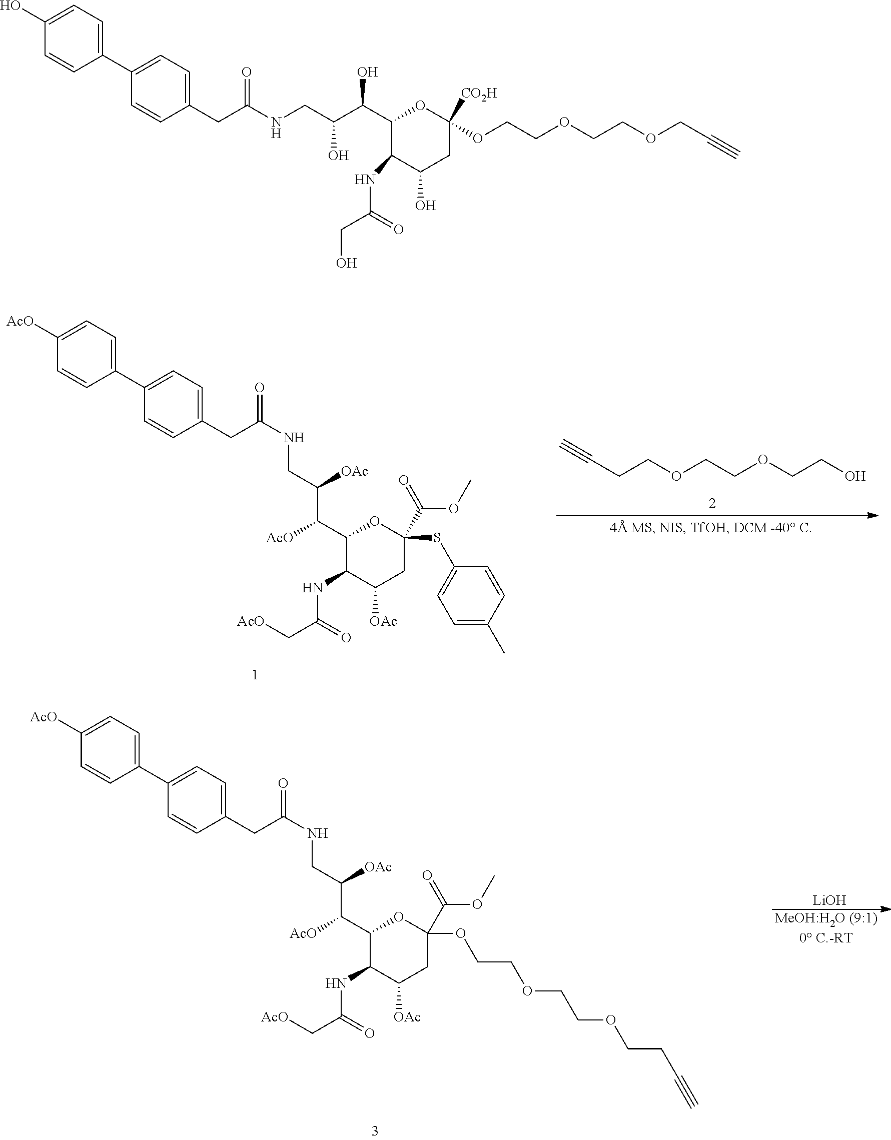

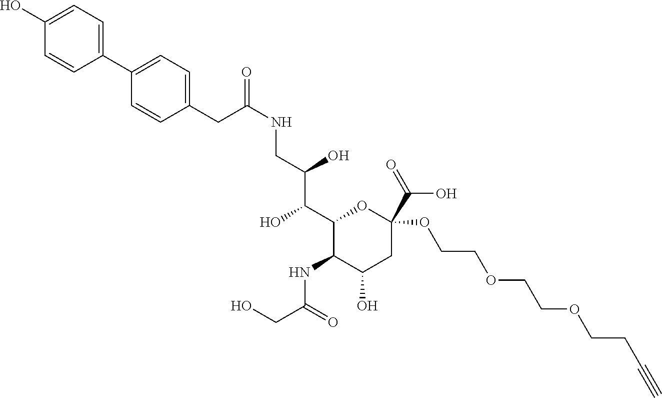 [SMILES: C#CCCOCCOCCO[C@]1(C(=O)O)C[C@H](O)[C@@H](NC(=O)CO)[C@H]([C@H](O)[C@H](O)CNC(=O)Cc2ccc(-c3ccc(O)cc3)cc2)O1]

### Synthesis of (1R,2R)-1-((2R,3R,4S)-4-acetoxy-3-(2-acetoxyacetamido)-6-(2-(2-(but-3-yn-1-yloxy)ethoxy)ethoxy)-6-(methoxycarbonyl)tetrahydro-2H-pyran-2-yl)-3-(2-(4′-acetoxy-[1,1′-biphenyl]-4-yl)acetamido)propane-1,2-diyl diacetate (3)

A mixture of (1R,2R)-1-((2R,3R,4S,6R)-3-acetamido-4-acetoxy-6-(methoxycarbonyl)-6-(p-tolylthio)tetrahydro-2H-pyran-2-yl)-3-(3-phenoxybenzamido)propane-1,2-diyl diacetate (1, 1.00 g, 1.16 mmol), 2-(2-(but-3-yn-1-yloxy)ethoxy)ethan-1-ol (2, 0.416 g, 2.89 mmol) and activated powdered 4 Å molecular sieves (1.00 g, 100% w/w) in anhydrous dichloromethane (20.0 mL) was stirred at room temperature for 15 h. The solution was then cooled to −40° C. followed by addition of 1-iodopyrrolidine-2,5-dione (0.624 g, 3.20 mmol) and trifluoromethanesulfonic acid (0.163 mL, 1.33 mmol). The reaction mixture was stirred at this temperature for another 1 h. After completion, the reaction mixture was quenched by addition of triethyl amine (neutral pH) and warmed up gradually to room temperature. The reaction mixture was filtered, and the filtrate was washed with deionized water. The organic layer was separated, dried, filtered, concentrated, and the crude residue obtained was purified by silica gel column chromatography using ethyl acetate in hexane as eluents to afford the desired product (3). Yield: 0.845 g, 81.30%; LCMS m/z 899.91 [M+H]⁺.

### Synthesis of (2R,4S,5R,6R)-2-(2-(2-(but-3-yn-1-yloxy)ethoxy)ethoxy)-6-((1R,2R)-1,2-dihydroxy-3-(2-(4′-hydroxy-[1,1′-biphenyl]-4-yl)acetamido)propyl)-4-hydroxy-5-(2-hydroxyacetamido)tetrahydro-2H-pyran-2-carboxylic acid (Cpd. 83)

To a stirred solution of (1R,2R)-1-((2R,3R,4S)-4-acetoxy-3-(2-acetoxyacetamido)-6-(2-(2-(but-3-yn-1-yloxy)ethoxy)ethoxy)-6-(methoxycarbonyl)tetrahydro-2H-pyran-2-yl)-3-(2-(4′-acetoxy-[1,1′-biphenyl]-4-yl)acetamido)propane-1,2-diyl diacetate (3, 0.84 g, 0.934 mmol) in methanol:water (9:1, 10 mL), was added a solution of lithium hydroxide monohydrate (0.134 g, 5.61 mmol). The reaction mixture was stirred at room temperature for 6 h. After completion, the reaction mixture was treated with acidic resin (Dowex 50, H⁺) until pH ˜6 and the suspension was filtered. The filtrate was concentrated, and the crude residue obtained was purified by prep HPLC using acetonitrile in water (+0.1% TFA) as eluents to afford the desired product (Cpd. 83). Yield: 0.122 g, 19.35%; LCMS m/z 661.05 [M+H]⁺; ¹H NMR (400 MHz, methanol-d₄) δ 7.50 (d, J=8.0 Hz, 2H), 7.44 (d, J=8.0 Hz, 2H), 7.33 (d, J=8.0 Hz, 2H), 6.84 (d, J=8.0 Hz, 2H), 4.17 (d, J=2.0 Hz, 1H), 4.00 (s, 1H), 3.92-3.78 (m, 4H), 3.74-3.56 (m, 12H), 3.37 (d, J=10.0 Hz, 1H), 3.27-3.22 (m, 1H), 2.82 (t, J=2.4 Hz, 1H), 2.74-2.69 (m, 1H), 1.75 (t, J=12.4 Hz, 1H).

### (2R,4S,5R,6R)-6-((1R,2R)-1,2-dihydroxy-3-(3-(4-hydroxyphenoxy)benzamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 18)

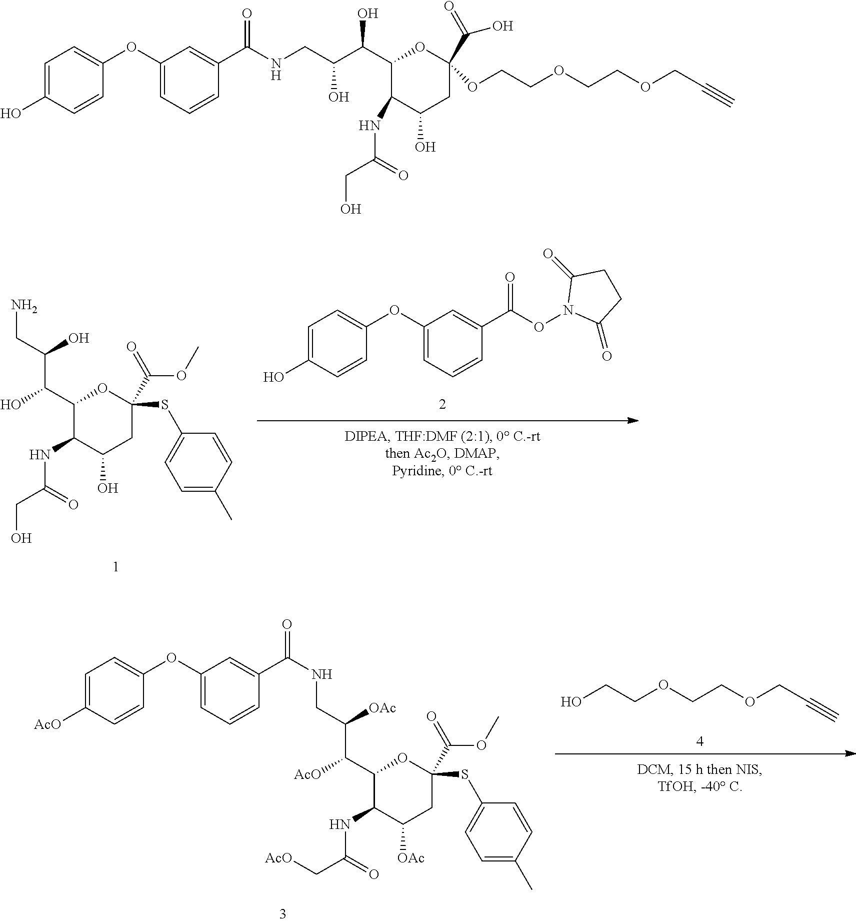

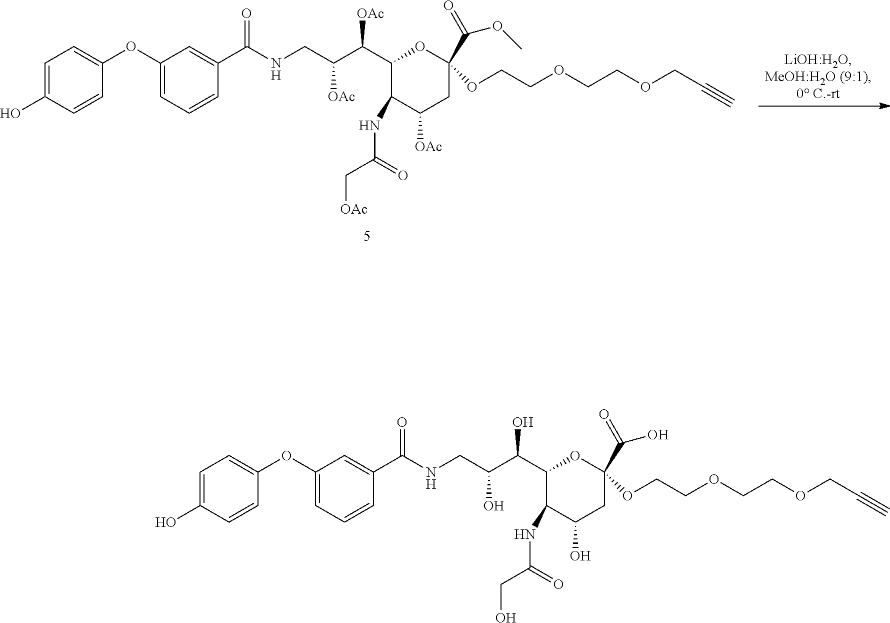 [SMILES: C#CCOCCOCCO[C@]1(C(=O)OC)C[C@H](OC(C)=O)[C@@H](NC(=O)COC(C)=O)[C@H]([C@H](OC(C)=O)[C@@H](CNC(=O)c2cccc(Oc3ccc(O)cc3)c2)OC(C)=O)O1 ; "LiOH:H2O,MeOH:H2O (9:1),0° C.-rt" ; C#CCOCCOCCO[C@]1(C(=O)O)C[C@H](O)[C@@H](NC(=O)CO)[C@H]([C@H](O)[C@H](O)CNC(=O)c2cccc(Oc3ccc(O)cc3)c2)O1]

### Synthesis of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(3-(4-acetoxyphenoxy)benzamido)propane-1,2-diyl diacetate (3)

To a stirred solution of methyl (2R,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (1, 1.0 g, 2.25 mmol) and 2,5-dioxopyrrolidin-1-yl 3-(4-hydroxyphenoxy)benzoate (2, 0.736 mg, 2.25 mmol) in tetrahydrofuran (10.00 mL) and N,N-dimethylformamide (2.00 mL), was added ethylbis(propan-2-yl)amine (1.18 mL, 6.75 mmol) at 0° C. then stirred at room temperature for 4 h. Acetic anhydride (2.13 mL, 22.50 mmol) and 4-dimethylaminopyridine (0.027 g, 0.22 mmol) was added at 0° C. and the resulting mixture was stirred at room temperature for 12 h. After completion the mixture was diluted with water and ethyl acetate. The organic layer was separated, dried, filtered, concentrated, and the crude residue obtained was purified by silica gel column chromatography using ethyl acetate in hexane as eluents to afford the desired product (3). Yield: 0.80 g, 43.11%; LCMS (ESI) m/z 867.26 [M+H]⁺.

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(3-(4-hydroxyphenoxy)benzamido)propane-1,2-diyl diacetate (5)

A mixture of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(3-(4-acetoxyphenoxy)benzamido)propane-1,2-diyl diacetate (3, 0.80 g, 0.97 mmol), 2-(2-(prop-2-yn-1-yloxy)ethoxy)ethan-1-ol (4, 0.349 g, 2.42 mmol) and activated powdered 4 Å molecular sieves (1.00 g, 100% w/w) in anhydrous dichloromethane (15.0 mL) was stirred at room temperature for 15 h. The solution was then cooled to −40° C. followed by addition of 1-iodopyrrolidine-2,5-dione (0.523 g, 2.33 mmol) and trifluoromethanesulfonic acid (0.082 mL, 0.97 mmol). The reaction mixture was stirred at this temperature for another 2 h. After completion, the reaction mixture was quenched by addition of triethyl amine (neutral pH) and warmed up gradually to room temperature. The reaction mixture was filtered, and the filtrate was washed with deionized water. The organic layer was separated, dried, filtered, concentrated, and the crude residue obtained was purified by silica gel column chromatography using ethyl acetate in hexane as eluents to afford the desired product (5) as a mixture of anomers. Yield: 0.700 g, 81.38%; LCMS m/z 903.70 [M+H₃O]⁺.

### Synthesis of (2R,4S,5R,6R)-6-((1R,2R)-1,2-dihydroxy-3-(3-(4-hydroxyphenoxy)benzamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 18)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(3-(4-hydroxyphenoxy)benzamido)propane-1,2-diyl diacetate (5, 0.70 g, 0.828 mmol) in methanol (10.0 mL), was added a solution of lithium hydroxide monohydrate (0.119 g, 4.97 mmol) in water (0.5 mL). The reaction mixture was stirred at room temperature for 4 h. After completion, the reaction mixture was treated with acidic resin (Dowex 50H⁺) until pH ˜6 and the suspension was filtered. The filtrate was concentrated, and the crude residue obtained was purified by prep HPLC using acetonitrile in water (+0.1% TFA) as eluents to afford the desired product (Cpd. 18). Yield: 0.058 g, 10.56%; LCMS m/z 663.40 [M+H]⁺; ¹H NMR (400 MHz, methanol-d₄) δ 7.47 (d, J=7.6 Hz, 1H), 7.39-7.35 (m, 2H), 7.05-7.03 (m, 1H), 6.90-6.87 (m, 2H), 6.82-6.78 (m, 2H), 4.17 (d, J=2.4 Hz, 2H), 4.05-3.75 (m, 8H), 3.66-3.60 (m, 7H), 3.51-3.42 (m, 2H), 2.81 (t, J=2.4 Hz, 1H), 2.73 (dd, J=12.8 & 3.6 Hz, 1H), 1.79-1.73 (m, 1H).

### (2R,4S,5R,6R)-6-((1R,2R)-1,2-dihydroxy-3-(4-(3-hydroxyphenoxy)benzamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 20)

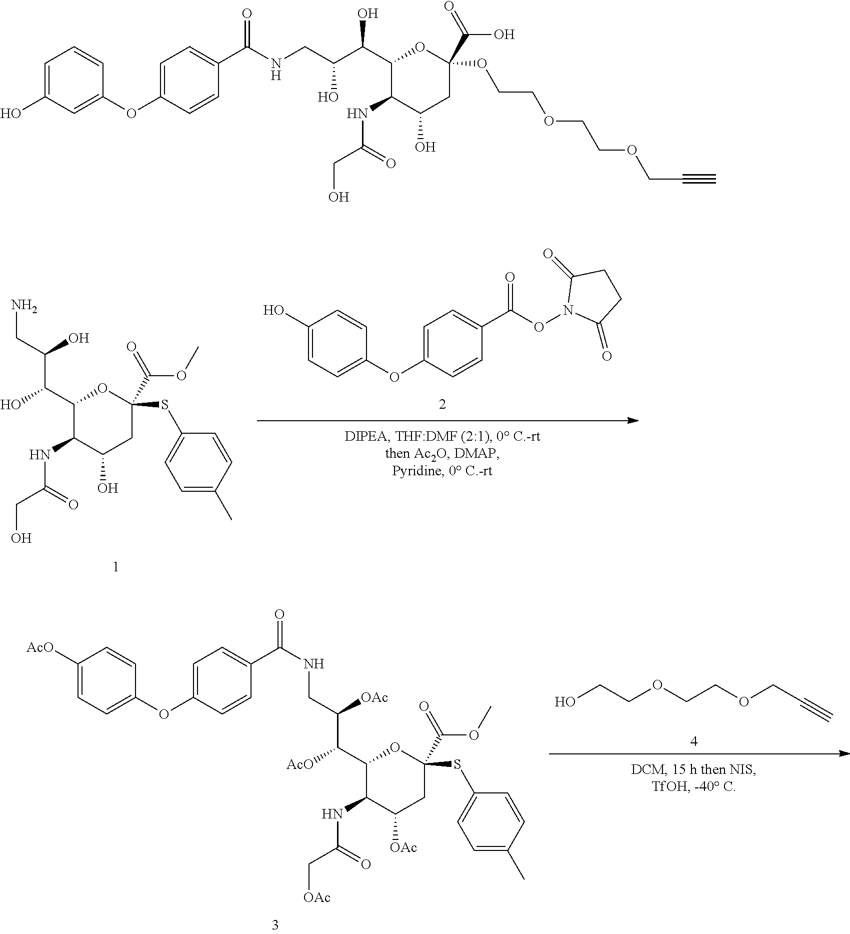

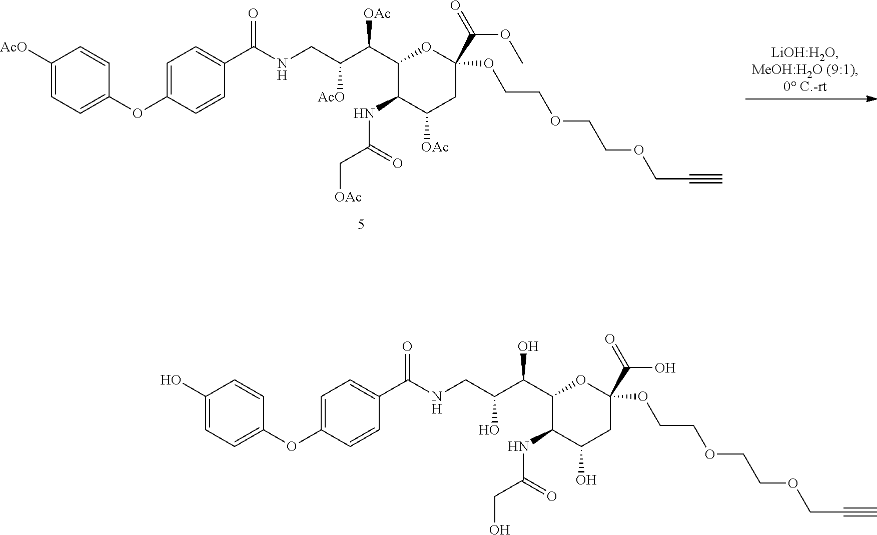 [SMILES: C#CCOCCOCCO[C@]1(C(=O)OC)C[C@H](OC(C)=O)[C@@H](NC(=O)COC(C)=O)[C@H]([C@H](OC(C)=O)[C@@H](CNC(=O)c2ccc(Oc3ccc(OC(C)=O)cc3)cc2)OC(C)=O)O1 ; "LiOH:H2O,MeOH:H2O (9:1),0° C.-rt" ; C#CCOCCOCCO[C@]1(C(=O)O)C[C@H](O)[C@@H](NC(=O)CO)[C@H]([C@H](O)[C@H](O)CNC(=O)c2ccc(Oc3ccc(O)cc3)cc2)O1]

### Synthesis of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(4-(4-hydroxyphenoxy)benzamido)propane-1,2-diyl diacetate (3)

To a stirred solution of methyl (2R,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (1, 1.50 g, 3.37 mmol) and 2,5-dioxopyrrolidin-1-yl 4-(4-hydroxyphenoxy)benzoate (2, 1.100 mg, 3.37 mmol) in tetrahydrofuran (15.00 mL) and N,N-dimethylformamide (2.00 mL), was added ethylbis(propan-2-yl)amine (1.76 mL, 10.12 mmol) at 0° C. then stirred at room temperature for 4 h. Acetic anhydride (3.19 mL, 33.75 mmol) and 4-dimethylaminopyridine (0.041 g, 0.337 mmol) was added at 0° C. and the resulting mixture was stirred at room temperature for 12 h. After completion the mixture was diluted with water and ethyl acetate. The organic layer was separated, dried, filtered, concentrated, and the crude residue obtained was purified by silica gel column chromatography using ethyl acetate in hexane as eluents to afford the desired product (3). Yield: 0.80 g, 28.74%; LCMS (ESI) m/z 866.65 [M+H]⁺.

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(4-(4-hydroxyphenoxy)benzamido)propane-1,2-diyl diacetate (5)

A mixture of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(4-(4-hydroxyphenoxy)benzamido)propane-1,2-diyl diacetate (3, 0.800 g, 0.97 mmol), 2-(2-(prop-2-yn-1-yloxy)ethoxy)ethan-1-ol (4, 0.310 g, 2.42 mmol) and activated powdered 4 Å molecular sieves (1.00 g, 100% w/w) in anhydrous dichloromethane (15.0 mL) was stirred at room temperature for 15 h. The solution was then cooled to −40° C. followed by addition of 1-iodopyrrolidine-2,5-dione (0.523 g, 2.33 mmol) and trifluoromethanesulfonic acid (0.082 mL, 0.97 mmol). The reaction mixture was stirred at this temperature for another 2 h. After completion, the reaction mixture was quenched by addition of triethyl amine (neutral pH) and warmed up gradually to room temperature. The reaction mixture was filtered, and the filtrate was washed with deionized water. The organic layer was separated, dried, filtered, concentrated, and the crude residue obtained was purified by silica gel column chromatography using ethyl acetate in hexane as eluents to afford the desired product (5) as a mixture of anomers. Yield: 0.750 g, 87.20%; LCMS m/z 903.70 [M+H₃O]⁺.

### Synthesis of (2R,4S,5R,6R)-6-((1R,2R)-1,2-dihydroxy-3-(4-(3-hydroxyphenoxy)benzamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 20)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(4-(4-hydroxyphenoxy)benzamido)propane-1,2-diyl diacetate (5, 0.75 g, 0.85 mmol) in methanol (10.0 mL), was added a solution of lithium hydroxide monohydrate (0.121 g, 5.07 mmol) in water (0.5 mL). The reaction mixture was stirred at room temperature for h. After 4 h. After completion, the reaction mixture was treated with acidic resin (Dowex 50H⁺) until pH˜6 and the suspension was filtered. The filtrate was concentrated, and the crude residue obtained was purified by prep HPLC using acetonitrile in water (+0.1% TFA) as eluents to afford the desired product (Cpd. 20). Yield: 0.045 g, 8.03%; LCMS m/z 663.50 [M+H]⁺; ¹H NMR (400 MHz, methanol-d₄) δ 7.79 (d, J=8.8 Hz, 2H), 6.93-6.89 (m, 4H), 6.84-6.81 (m, 2H), 4.18 (d, J=2.4 Hz, 2H), 4.17-4.12 (m, 3H), 4.05-3.90 (m, 5H), 3.85-3.76 (m, 2H), 3.69-3.63 (m, 7H), 3.53-3.48 (m, 1H), 3.43 (d, J=9.2 Hz, 1H), 2.81 (t, J=2.4 Hz, 1H), 2.72 (dd, J=12.8 & 4.0 Hz, 1H), 1.76 (t, J=12.0 Hz, 1H).

### (2R,4S,5R,6R)-6-((1R,2R)-1,2-dihydroxy-3-(4′-hydroxy-[1,1′-biphenyl]-4-carboxamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 19)

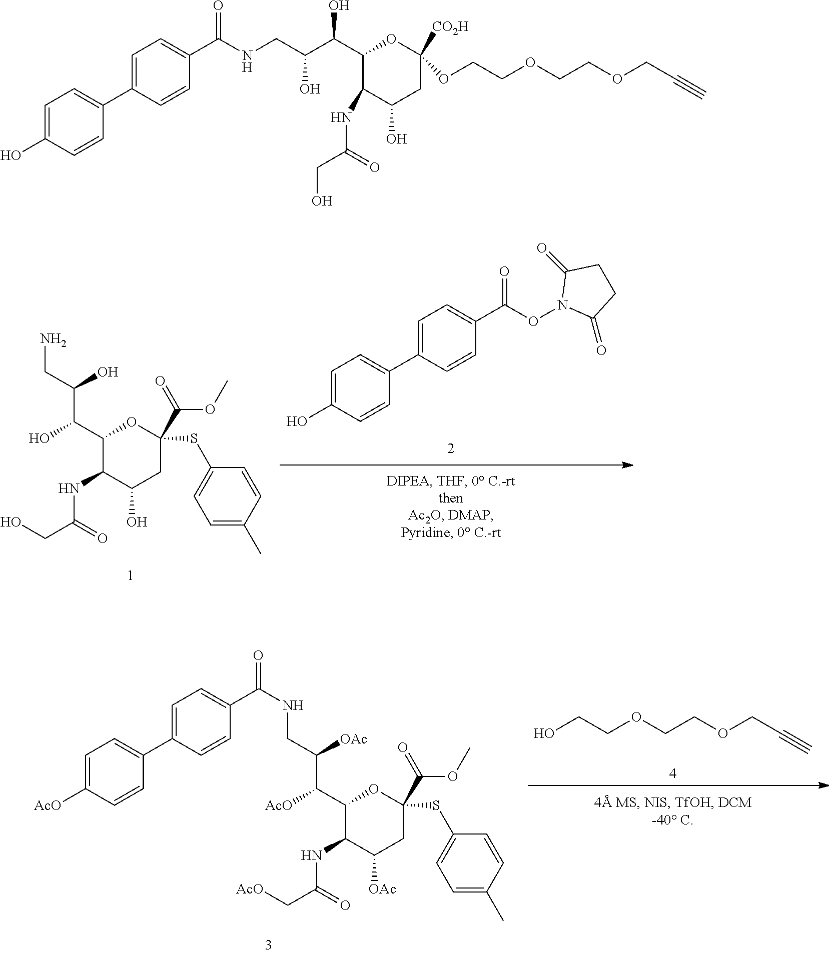

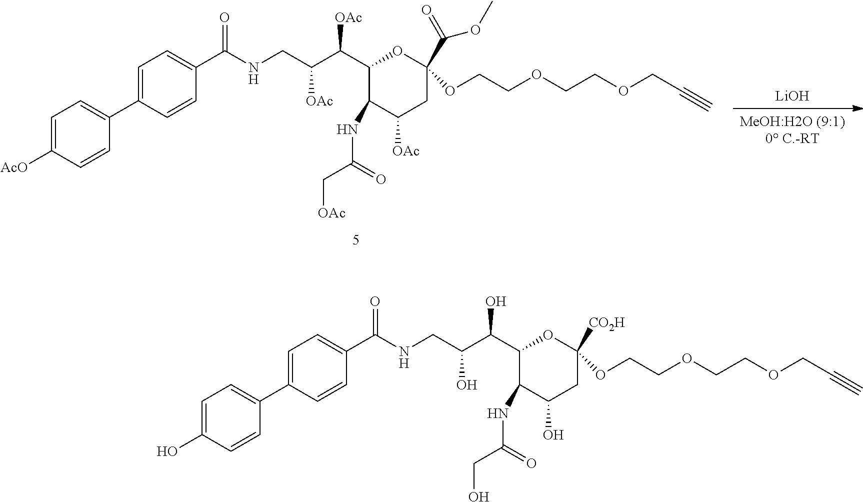 [SMILES: C#CCOCCOCCO[C@]1(C(=O)OC)C[C@H](OC(C)=O)[C@@H](NC(=O)COC(C)=O)[C@H]([C@H](OC(C)=O)[C@@H](CNC(=O)c2ccc(-c3ccc(OC(C)=O)cc3)cc2)OC(C)=O)O1 ; "LiOH" ; "MeOH:H2O (9:1)0° C.-RT" ; C#CCOCCOCCO[C@]1(C(=O)O)C[C@H](O)[C@@H](NC(=O)CO)[C@H]([C@H](O)[C@H](O)CNC(=O)c2ccc(-c3ccc(O)cc3)cc2)O1]

### Synthesis of (1R,2R)-1-((2R,3R,4S,6S)-4-acetoxy-3-(2-acetoxyacetamido)-6-(methoxycarbonyl)-6-(p-tolylthio)tetrahydro-2H-pyran-2-yl)-3-(4′-acetoxy-[1,1′-biphenyl]-4-carboxamido)propane-1,2-diyl diacetate (3)

To a stirred solution of methyl (2S,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (1, 1.30 g, 2.92 mmol) and 2,5-dioxopyrrolidin-1-yl 4′-hydroxy-[1,1′-biphenyl]-4-carboxylate (2, 0.910 g, 2.92 mmol) in tetrahydrofuran (10.0 mL) and N,N-dimethylformamide (2.00 mL), was added ethylbis(propan-2-yl)amine (1.43 mL, 8.77 mmol) at 0° C. then stirred at room temperature for 4 h. Acetic anhydride (2.21 mL, 23.40 mmol) and 4-dimethylaminopyridine (0.035 g, 0.29 mmol) was added at 0° C. and the resulting mixture was stirred at room temperature for 12 h. After completion the mixture was diluted with water and ethyl acetate. The organic layer was separated, dried, filtered, concentrated, and the crude residue obtained was purified by silica gel column chromatography using ethyl acetate in hexane as eluents to afford the desired product (3). Yield: 0.32 g, 12.86%; LCMS (ESI) m/z 850.70 [M+H]⁺.

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(4′-acetoxy-[1,1′-biphenyl]-4-carboxamido)propane-1,2-diyl diacetate (5)

A mixture of (1R,2R)-1-((2R,3R,4S,6S)-4-acetoxy-3-(2-acetoxyacetamido)-6-(methoxycarbonyl)-6-(p-tolylthio)tetrahydro-2H-pyran-2-yl)-3-(4′-acetoxy-[1,1′-biphenyl]-4-carboxamido)propane-1,2-diyl diacetate (3, 0.32 g, 0.376 mmol), 2-(2-(prop-2-yn-1-yloxy)ethoxy)ethan-1-ol (4, 0.135 g, 0.940 mmol) and activated powdered 4 Å molecular sieves (0.50 g, 100% w/w) in anhydrous dichloromethane (10.0 mL) was stirred at room temperature for 15 h. The solution was then cooled to −40° C. followed by addition of 1-iodopyrrolidine-2,5-dione (0.203 g, 0.902 mmol) and trifluoromethanesulfonic acid (0.031 mL, 0.376 mmol). The reaction mixture was stirred at this temperature for another 2 h. After completion, the reaction mixture was quenched by addition of triethyl amine (neutral pH) and warmed up gradually to room temperature. The reaction mixture was filtered, and the filtrate was washed with deionized water. The organic layer was separated, dried, filtered, concentrated, and the crude residue obtained was purified by silica gel column chromatography using ethyl acetate in hexane as eluents to afford the desired product (5) as a mixture of anomers. Yield: 0.25 g, 76.33%; LCMS m/z 870.80 [M+H]⁺.

### Synthesis of (2R,4S,5R,6R)-6-((1R,2R)-1,2-dihydroxy-3-(4′-hydroxy-[1,1′-biphenyl]-4-carboxamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 19)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(4′-acetoxy-[1,1′-biphenyl]-4-carboxamido)propane-1,2-diyl diacetate (5, 0.25 g, 0.287 mmol) in methanol (5.0 mL), was added a solution of lithium hydroxide monohydrate (0.070 g, 1.72 mmol) in water (0.5 mL). The reaction mixture was stirred at room temperature for 4 h. After completion, the reaction mixture was treated with acidic resin (Dowex 50H⁺) until pH˜6 and the suspension was filtered. The filtrate was concentrated, and the crude residue obtained was purified by prep HPLC using acetonitrile in water (+0.1% TFA) as eluents to afford the desired product (Cpd. 19). Yield: 0.010 g, 5.39%; LCMS m/z 647.30 [M+H]⁺; ¹H NMR (400 MHz, methanol-d₄) δ 7.87 (d, J=8.4 Hz, 2H), 7.65 (d, J=8.4 Hz, 2H), 7.52 (d, J=8.8 Hz, 2H), 6.87 (d, J=8.8 Hz, 2H), 4.17 (d, J=2.4 Hz, 2H), 4.06-4.01 (m, 3H), 3.98-3.93 (m, 1H), 3.87-3.78 (m, 4H), 3.64-3.61 (m, 7H), 3.56-3.51 (m, 1H), 3.48-3.45 (m, 1H), 2.81 (t, J=2.4 Hz, 1H), 2.76-2.72 (m, 1H), 1.79-1.73 (m, 1H).

### (2R,4S,5R,6R)-6-((1R,2R)-1,2-dihydroxy-3-(2-(4′-hydroxy-[1,1′-biphenyl]-4-yl)acetamido)propyl)-4-hydroxy-5-(2-hydroxyacetamido)-N-(methylsulfonyl)-2-(2-(2-(prop-2-yn-1-yloxy)ethoxy)ethoxy)tetrahydro-2H-pyran-2-carboxamide (Compound. 65)

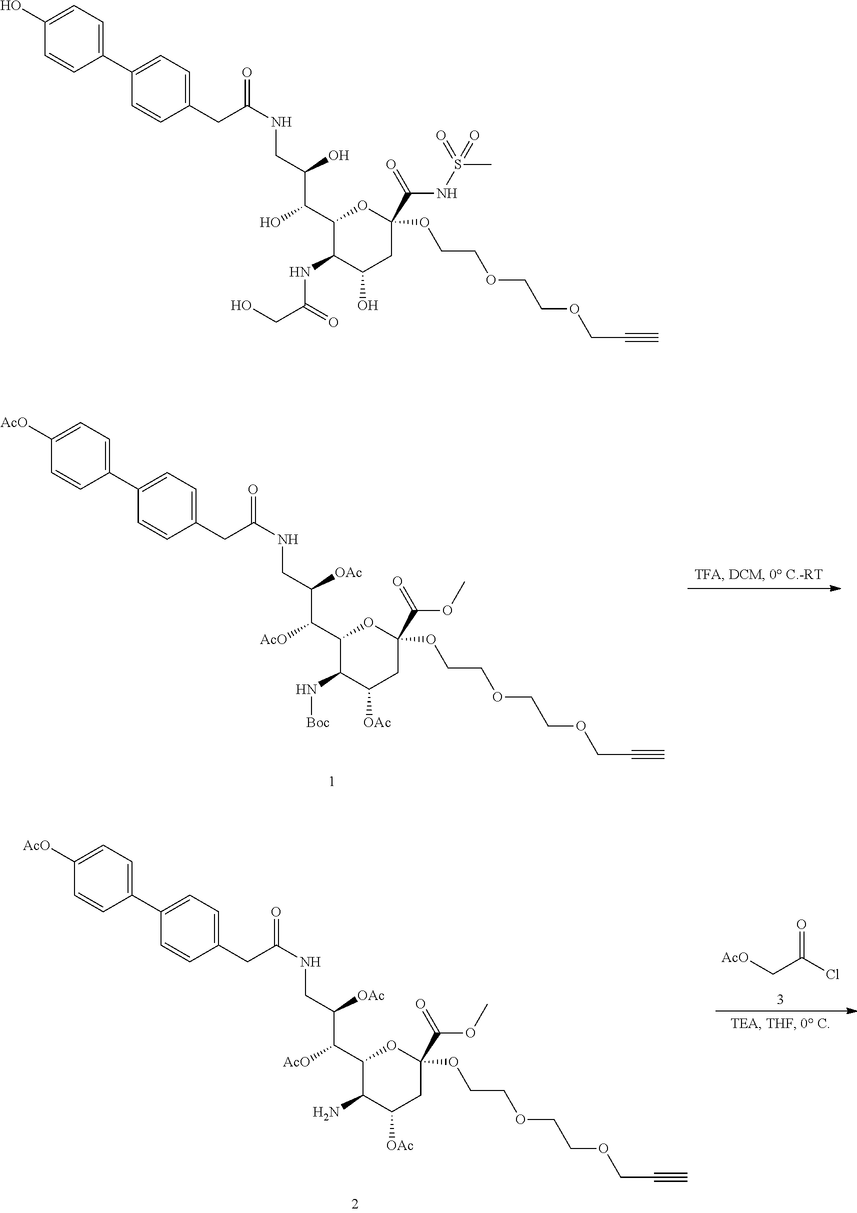

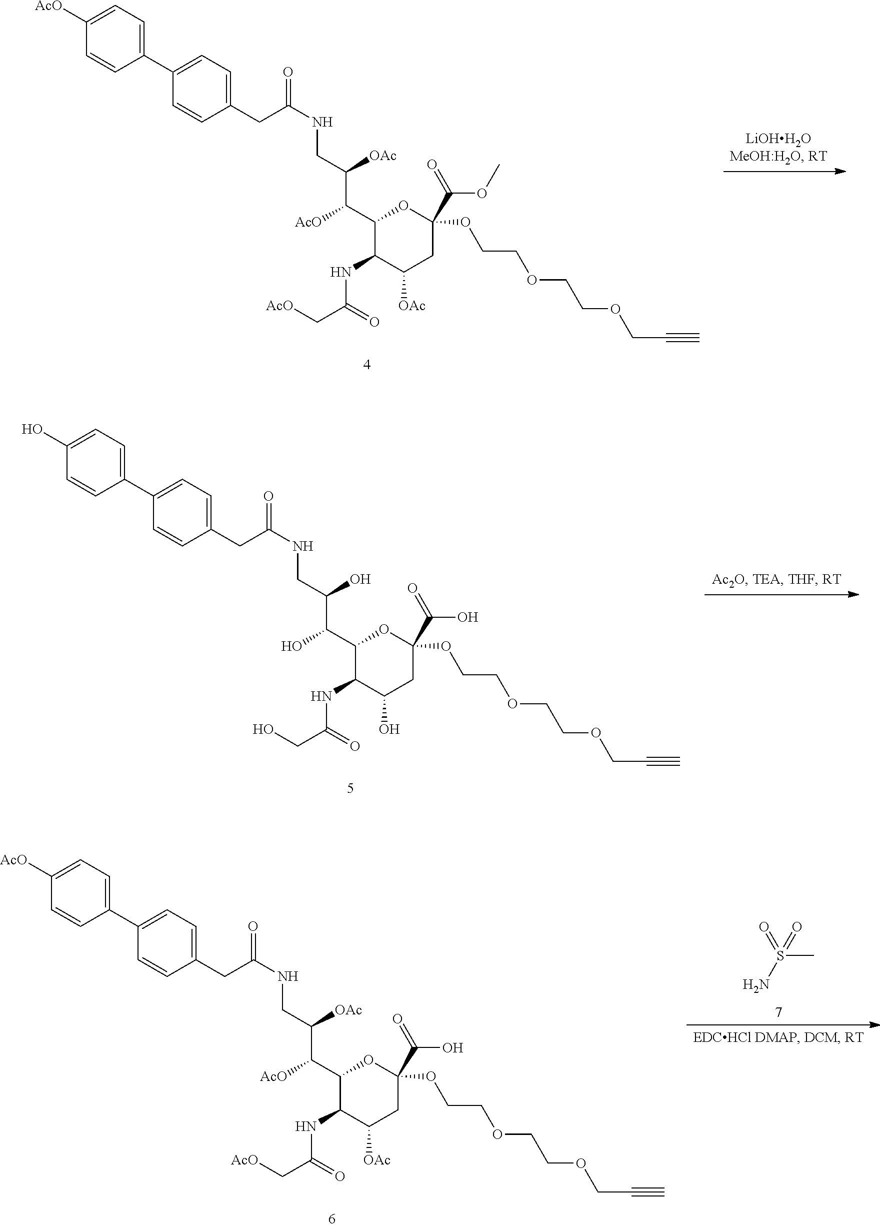

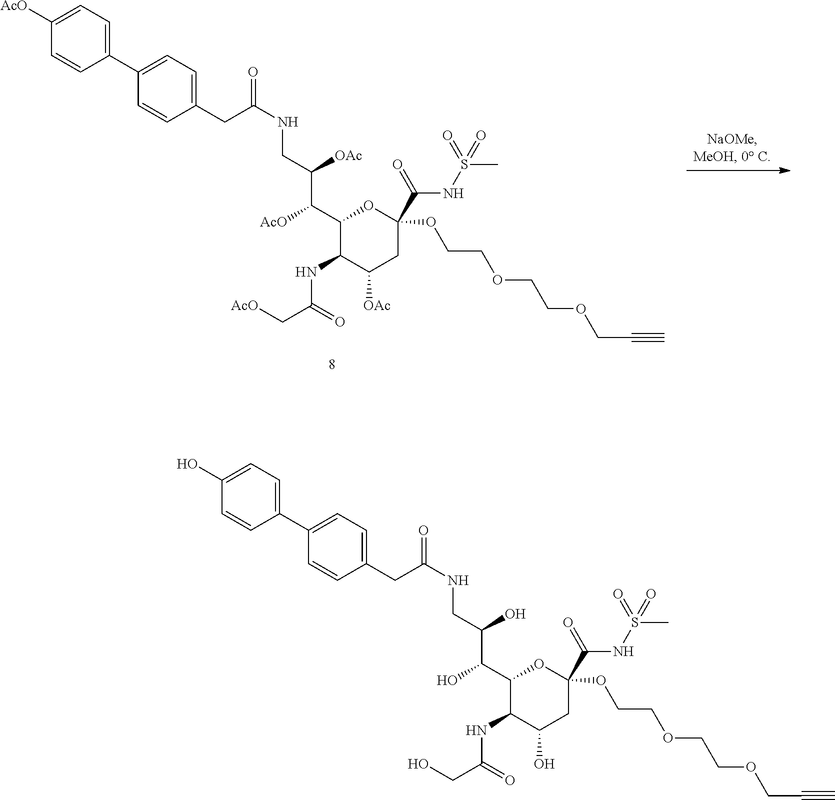 [SMILES: C#CCOCCOCCO[C@]1(C(=O)NS(C)(=O)=O)C[C@H](OC(C)=O)[C@@H](NC(=O)COC(C)=O)[C@H]([C@H](OC(C)=O)[C@@H](CNC(=O)Cc2ccc(-c3ccc(OC(C)=O)cc3)cc2)OC(C)=O)O1 ; "NaOMe,MeOH, 0° C." ; C#CCOCCOCCO[C@]1(C(=O)NS(C)(=O)=O)C[C@H](O)[C@@H](NC(=O)CO)[C@H]([C@H](O)[C@H](O)CNC(=O)Cc2ccc(-c3ccc(O)cc3)cc2)O1]

### Synthesis of (1R,2R)-1-((2R,3R,4S,6R)-4-acetoxy-3-amino-6-(methoxycarbonyl)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (2)

To a stirred solution of (1R,2R)-1-((2R,3R,4S,6R)-4-acetoxy-3-((tert-butoxycarbonyl)amino)-6-(methoxycarbonyl)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (1, 0.50 g, 0.565 mmol) in dichloromethane (5.0 mL) was added trifluoroacetic acid (2.0 mL) at 0° C. then stirred at room temperature for 2 h. After completion, the reaction mixture was concentrated, and the crude residue obtained was purified by trituration with diethyl ether to afford the desired product (2). Yield: 0.50 g; LCMS m/z 785.55 [M+H]⁺.

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (4)

(1R,2R)-1-((2R,3R,4S,6R)-4-acetoxy-3-amino-6-(methoxycarbonyl)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (2, 0.50 g, 0.637 mmol) was dissolved under stirring in dry tetrahydrofuran (5.0 mL) and cooled at 0° C. To this solution 2-chloro-2-oxoethyl acetate (0.069 g, 0.509 mmol) was added slowly followed by triethylamine (0.44 mL, 3.18 mmol) at 0° C. After completion the mixture was concentrated, and the crude residue obtained was purified by silica gel column chromatography using ethyl acetate in heptane as eluents to afford the desired product (4). Yield: 0.30 g, 53%; LCMS (ESI) m/z 882.90 [M−H]⁻.

### Synthesis of (2R,4S,5R,6R)-6-((1R,2R)-1,2-dihydroxy-3-(2-(4′-hydroxy-[1,1′-biphenyl]-4-yl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (5)

To a stirred solution (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (4, 0.30 g, 0.395 mmol) in methanol (3.0 mL) and water (0.5 mL), was added lithium hydroxide monohydrate (0.101 g, 2.35 mmol) at 0° C. The reaction mixture was stirred at room temperature for 6 h. After completion, the reaction mixture was treated with acidic resin (Dowex 50, H⁺) to pH ˜6 and the suspension was filtered. The filtrate was concentrated, and the crude residue obtained was purified by trituration with diethyl ether to afford the desired product (5). Yield: 0.200 g, 77%; LCMS m/z 661.20 [M+H]⁺.

### Synthesis of (2R,4S,5R,6R)-4-acetoxy-5-(2-acetoxyacetamido)-6-((1R,2R)-1,2-diacetoxy-3-(2-(4′-acetoxy-[1,1′-biphenyl]-4-yl)acetamido)propyl)-2-(2-(2-(prop-2-yn-1-yloxy)ethoxy)ethoxy)tetrahydro-2H-pyran-2-carboxylic acid (6)

To a stirred solution of (2R,4S,5R,6R)-6-((1R,2R)-1,2-dihydroxy-3-(2-(4′-hydroxy-[1,1′-biphenyl]-4-yl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (5, 0.130 g, 0.197 mmol) in tetrahydrofuran (2.0 mL) were added trimethyl amine (0.27 mL, 1.97 mmol), 4-(dimethylamino) pyridine (0.0024 g, 0.019 mmol) and acetic anhydride (0.18 mL, 1.97 mmol) at 0° C. and allowed to stir for 12 h at room temperature. After completion, the reaction mixture was concentrated. The residue obtained was taken up in ethyl acetate, washed with aqueous saturated solution of sodium bicarbonate, once with water, and twice with brine. the The organic layer was dried, filtered, and filtrate was concentrated, and the crude residue was purified by silica gel column chromatography using ethyl acetate in heptane as eluents to afford the desired product (6). Yield: 0.110 g, 64%; LCMS m/z 868.85 [M−H]⁻.

### Synthesis of (1R,2R)-1-((2R,3R,4S,6R)-4-acetoxy-3-(2-acetoxyacetamido)-6-((methylsulfonyl)carbamoyl)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (7)

To a stirred solution of (2R,4S,5R,6R)-4-acetoxy-5-(2-acetoxyacetamido)-6-((1R,2R)-1,2-diacetoxy-3-(2-(4′-acetoxy-[1,1′-biphenyl]-4-yl)acetamido)propyl)-2-(2-(2-(prop-2-yn-1-yloxy)ethoxy)ethoxy)tetrahydro-2H-pyran-2-carboxylic acid (6, 0.100 g, 0.114 mmol) in dichloromethane (2.0 mL) were added N-(3-Dimethylaminopropyl)-N′-ethylcarbodiimide hydrochloride (0.071 g, 0.460 mmol), 4-(dimethylamino) pyridine (0.0028 g, 0.228 mmol) and methanesulfonamide (0.021 g, 0.228 μmol) at 0° C. and stirred at room temperature for 12 h. After completion, the reaction mixture was concentrated. The residue was purified by silica gel column chromatography using methanol in dichloromethane to afford the desired product (7). Yield: 0.100 g, 91%; LCMS m/z 945.85 [M−H]⁻.

### Synthesis of (2R,4S,5R,6R)-6-((1R,2R)-1,2-dihydroxy-3-(2-(4′-hydroxy-[1,1′-biphenyl]-4-yl)acetamido)propyl)-4-hydroxy-5-(2-hydroxyacetamido)-N-(methylsulfonyl)-2-(2-(2-(prop-2-yn-1-yloxy)ethoxy)ethoxy)tetrahydro-2H-pyran-2-carboxamide (Cpd. 65)

To a stirred solution of (1R,2R)-1-((2R,3R,4S,6R)-4-acetoxy-3-(2-acetoxyacetamido)-6-((methylsulfonyl)carbamoyl)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (7, 0.10 g, 0.105 mmol) in methanol (2.0 mL) was added sodium methoxide solution (25% in methanol, 0.005 ml, 0.021 mmol) slowly at 0° C. The reaction mixture was stirred at room temperature for 2 h. After completion, the reaction mixture was treated with acidic resin (Dowex 50, H⁺) to pH ˜6 and the suspension was filtered. The filtrate was concentrated, and the crude residue obtained was purified by prep HPLC using acetonitrile in water (+0.1% TFA) as eluents to afford the desired product (Cpd. 65). Yield: 0.001 g, 1.28%; LCMS m/z 581.40 [M+H]⁺. ¹H NMR (400 MHz, MeOD-d4): δ 8.07 (d, J=8.4 Hz, 1H), 7.91 (d, J=7.6 Hz, 1H), 7.50 (d, J=8.0 Hz, 2H), 7.46-7.44 (m, 2H), 7.33 (d, J=8.0 Hz, 2H), 6.84 (d, J=8.4 Hz, 1H), 4.16 (d, J=2.4 Hz, 2H), 4.01 (s, 2H), 3.86-3.85 (m, 3H), 3.80-3.78 (m, 2H), 3.76-3.70 (m, 2H), 3.66-3.59 (m, 7H), 3.56 (s, 2H), 3.40-3.37 (m, 1H), 3.20-3.17 (m, 2H), 2.81 (t, J=2.4 Hz, 1H), 2.78 (m, 1H), 1.69 (m, 1H).

### (2R,4S,5R,6R)-6-((1R,2R)-1,2-dihydroxy-3-(2-(4-(pyridin-4-yl)phenyl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 72)

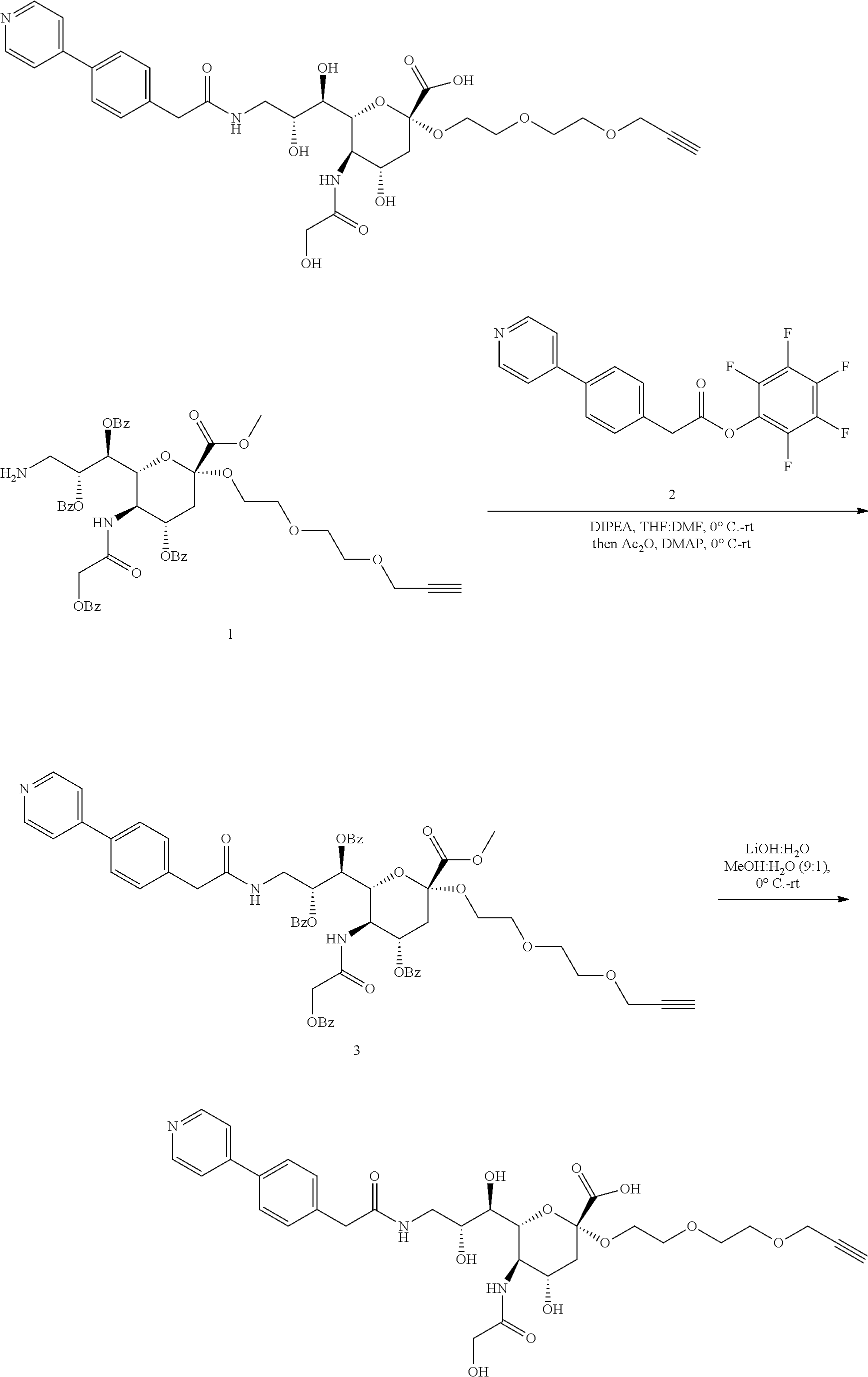

### Synthesis of (1R,2R)-1-((2R,3R,4S,6R)-4-(benzoyloxy)-3-(2-(benzoyloxy)acetamido)-6-(methoxycarbonyl)-6-(2-(2-(prop-2-yn-1-yloxy)ethoxy)ethoxy)tetrahydro-2H-pyran-2-yl)-3-(2-(4-(pyridin-4-yl)phenyl)acetamido)propane-1,2-diyl dibenzoate (3)

To a stirred solution of (1R,2R)-3-amino-1-((2R,3R,4S,6R)-4-(benzoyloxy)-3-(2-(benzoyloxy)acetamido)-6-(methoxycarbonyl)-6-(2-(2-(prop-2-yn-1-yloxy)ethoxy)ethoxy)tetrahydro-2H-pyran-2-yl)propane-1,2-diyl dibenzoate (1, 0.20 g, 0.20 mmol) and perfluorophenyl 2-(4-(pyridin-4-yl)phenyl)acetate (2, 0.077 g, 0.20 mmol) in tetrahydrofuran (5.00 mL) and N,N-dimethylformamide (1.0 mL), was added ethylbis(propan-2-yl)amine (0.106 mL, 0.61 mmol) at 0° C. then stirred at room temperature for 4 h. Acetic anhydride (0.154 mL, 1.64 mmol) and 4-dimethylaminopyridine (0.012 g, 0.02 mmol) was added at 0° C. and the resulting mixture was stirred at room temperature for 12 h. After completion the mixture was diluted with water and ethyl acetate. The organic layer was separated, dried, filtered, concentrated, and the crude residue obtained was purified by silica gel column chromatography using ethyl acetate in hexane as eluents to afford the desired product (3). Yield: 0.21 g, 95.42%; LCMS (ESI) m/z 1076.10 [M+H]⁺.

### Synthesis of (2R,4S,5R,6R)-6-((1R,2R)-1,2-dihydroxy-3-(2-(4-(pyridin-4-yl)phenyl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 72)

To a stirred solution of (1R,2R)-1-((2R,3R,4S,6R)-4-(benzoyloxy)-3-(2-(benzoyloxy)acetamido)-6-(methoxycarbonyl)-6-(2-(2-(prop-2-yn-1-yloxy)ethoxy)ethoxy)tetrahydro-2H-pyran-2-yl)-3-(2-(4-(pyridin-4-yl)phenyl)acetamido)propane-1,2-diyl dibenzoate (3, 0.21 g, 0.195 mmol) in methanol (5.0 mL), was added a solution of lithium hydroxide monohydrate (0.049 g, 1.17 mmol) in water (0.5 mL). The reaction mixture was stirred at room temperature for 4 h. After completion, the reaction mixture was treated with acidic resin (Dowex 50H⁺) until pH˜6 and the suspension was filtered. The filtrate was concentrated, and the crude residue obtained was purified by prep HPLC using acetonitrile and water (+0.1% TFA) as eluents to afford the desired product (Cpd. 72). Yield: 0.003 g, 2.38%; LCMS m/z 668.4 [M+Na]⁺; ¹H NMR (400 MHz, methanol-d₄) δ 8.67 (m, 2H), 7.99 (d, J=5.6 Hz, 2H), 7.82 (d, J=8.0 Hz, 2H), 7.51 (d, J=8.0 Hz, 2H), 4.17 (d, J=2.4 Hz, 2H), 4.00 (s, 2H), 3.91-3.58 (m, 15H), 3.36-3.23 (m, 2H), 2.83 (t, J=2.4 Hz, 1H), 2.74 (dd, J=12.8 & 4.0 Hz, 1H), 1.76-1.69 (m, 1H).

### (2R,4S,5R,6R)-6-((1R,2R)-1,2-dihydroxy-3-(2-(6-phenylpyridin-3-yl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 79)

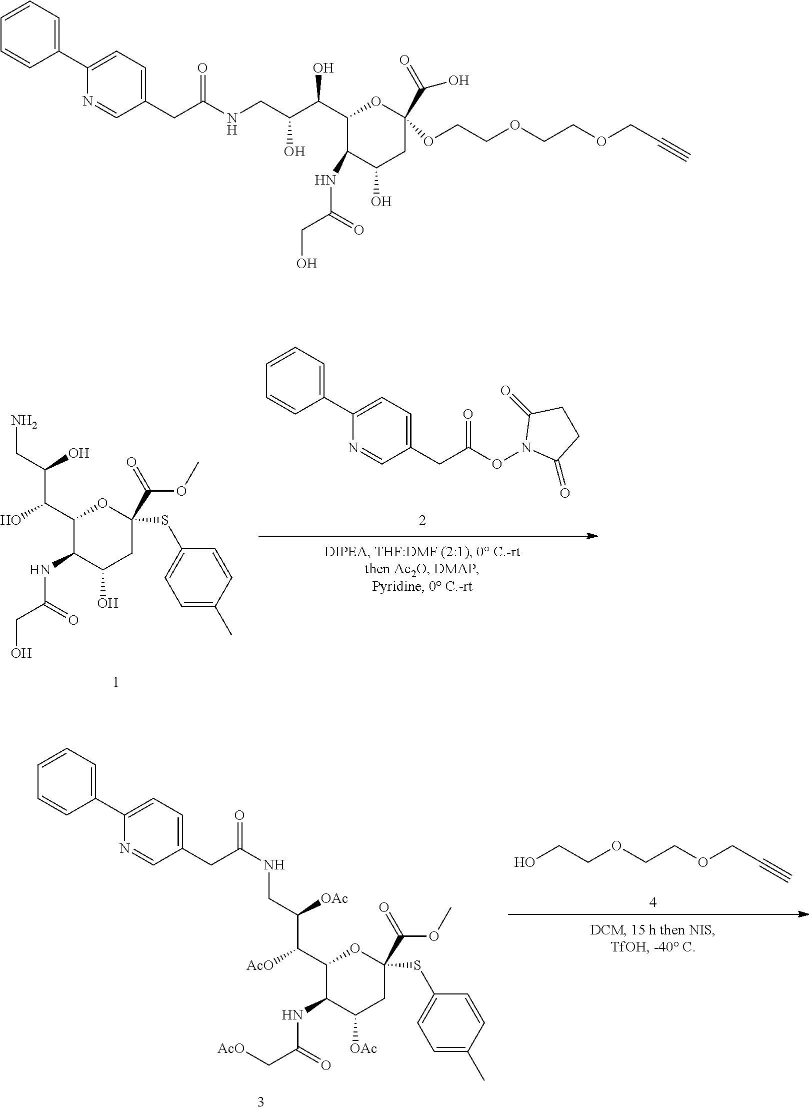

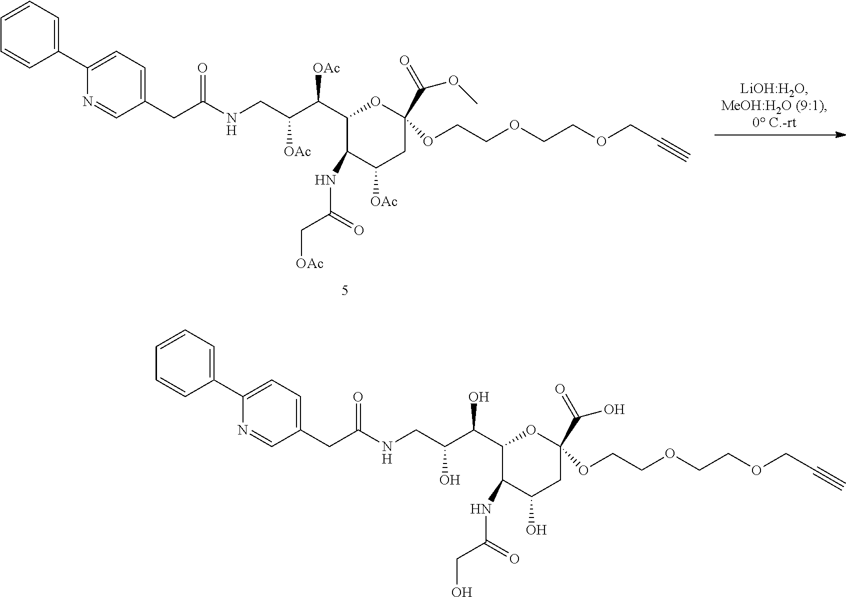 [SMILES: C#CCOCCOCCO[C@]1(C(=O)OC)C[C@H](OC(C)=O)[C@@H](NC(=O)COC(C)=O)[C@H]([C@H](OC(C)=O)[C@@H](CNC(=O)Cc2ccc(-c3ccccc3)nc2)OC(C)=O)O1 ; "LiOH:H2O,MeOH:H2O (9:1),0° C.-rt" ; C#CCOCCOCCO[C@]1(C(=O)O)C[C@H](O)[C@@H](NC(=O)CO)[C@H]([C@H](O)[C@H](O)CNC(=O)Cc2ccc(-c3ccccc3)nc2)O1]

### Synthesis of (1R,2R)-1-((2R,3R,4S,6S)-4-acetoxy-3-(2-acetoxyacetamido)-6-(methoxycarbonyl)-6-(p-tolylthio)tetrahydro-2H-pyran-2-yl)-3-(2-(6-phenylpyridin-3-yl)acetamido)propane-1,2-diyl diacetate (3)

To a stirred solution of methyl (2S,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (2, 0.838 g, 1.89 mmol) in tetrahydrofuran (10.00 mL) and N,N-dimethylformamide (2.00 mL), was added ethylbis(propan-2-yl)amine (1.69 mL, 9.43 mmol) at 0° C. then stirred at room temperature for 4 h. Acetic anhydride (1.69 mL, 9.43 mmol) and 4-dimethylaminopyridine (0.041 g, 0.34 mmol) was added at 0° C. and the resulting mixture was stirred at room temperature for 12 h. After completion the mixture was diluted with water and ethyl acetate. The organic layer was separated, dried, filtered, concentrated, and the crude residue obtained was purified by silica gel column chromatography using ethyl acetate in hexane as eluents to afford the desired product (3). Yield: 1.0 g, 65.66%; LCMS (ESI) m/z 808.12 [M+H]⁺.

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(6-phenylpyridin-3-yl)acetamido)propane-1,2-diyl diacetate (5)

A mixture of (1R,2R)-1-((2R,3R,4S,6S)-4-acetoxy-3-(2-acetoxyacetamido)-6-(methoxycarbonyl)-6-(p-tolylthio)tetrahydro-2H-pyran-2-yl)-3-(2-(6-phenylpyridin-3-yl)acetamido)propane-1,2-diyl diacetate (3, 1.0 g, 1.24 mmol), 2-(2-(prop-2-yn-1-yloxy)ethoxy)ethan-1-ol (4, 0.436 g, 3.09 mmol) and activated powdered 4 Å molecular sieves (1.00 g, 100% w/w) in anhydrous dichloromethane (15.0 mL) was stirred at room temperature for 15 h. The solution was then cooled to −40° C. followed by addition of 1-iodopyrrolidine-2,5-dione (0.697 g, 3.09 mmol) and trifluoromethanesulfonic acid (0.17 mL, 1.24 mmol). The reaction mixture was stirred at this temperature for another 2 h. After completion, the reaction mixture was quenched by addition of triethyl amine (neutral pH) and warmed up gradually to room temperature. The reaction mixture was filtered, and the filtrate was washed with deionized water. The organic layer was separated, dried, filtered, concentrated, and the crude residue obtained was purified by silica gel column chromatography using ethyl acetate in hexane as eluents to afford the desired product (5). Yield: 0.750 g, 73.19%; LCMS m/z 828.05 [M+H]⁺.

### Synthesis of (2R,4S,5R,6R)-6-((1R,2R)-1,2-dihydroxy-3-(2-(6-phenylpyridin-3-yl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 79)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(6-phenylpyridin-3-yl)acetamido)propane-1,2-diyl diacetate (5, 0.50 g, 0.604 mmol) in methanol (10.0 mL), was added a solution of lithium hydroxide monohydrate (0.148 g, 3.62 mmol) in water (0.5 mL). The reaction mixture was stirred at room temperature for h. After 4 h. After completion, the reaction mixture was treated with acidic resin (Dowex 50H⁺) until pH ˜6 and the suspension was filtered. The filtrate was concentrated, and the crude residue obtained was purified by prep HPLC using acetonitrile in water (+0.1% TFA) as eluents to afford the desired product (Cpd. 79). Yield: 0.133 g, 34.19%; LCMS m/z 646.30 [M+H]⁺; ¹H NMR (400 MHz, methanol-d₄) δ 8.72 (d, J=1.6 Hz, 1H), 8.41 (dd, J=8.4 & 2.0 Hz, 1H), 8.21 (d, J=8.4 Hz, 1H), 7.96-7.93 (m, 2H), 7.66-7.63 (m, 3H), 4.18 (d, J=2.4 Hz, 2H), 4.01 (s, 2H), 3.99-3.93 (m, 2H), 3.87-3.72 (m, 4H), 3.69-3.58 (m, 8H), 3.39 (dd, J=8.8 & 1.6 Hz, 1H), 3.35-3.31 (m, 1H), 2.94 (s, 1H), 2.84 (t, J=2.4 Hz, 1H), 2.73 (dd, J=12.8 & 4.0 Hz, 1H), 1.76 (t, J=11.6 Hz, 1H).

### (2R,4S,5R,6R)-6-((1R,2R)-1,2-dihydroxy-3-(2-(3′-(methylcarbamoyl)-[1,1′-biphenyl]-4-yl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 80)

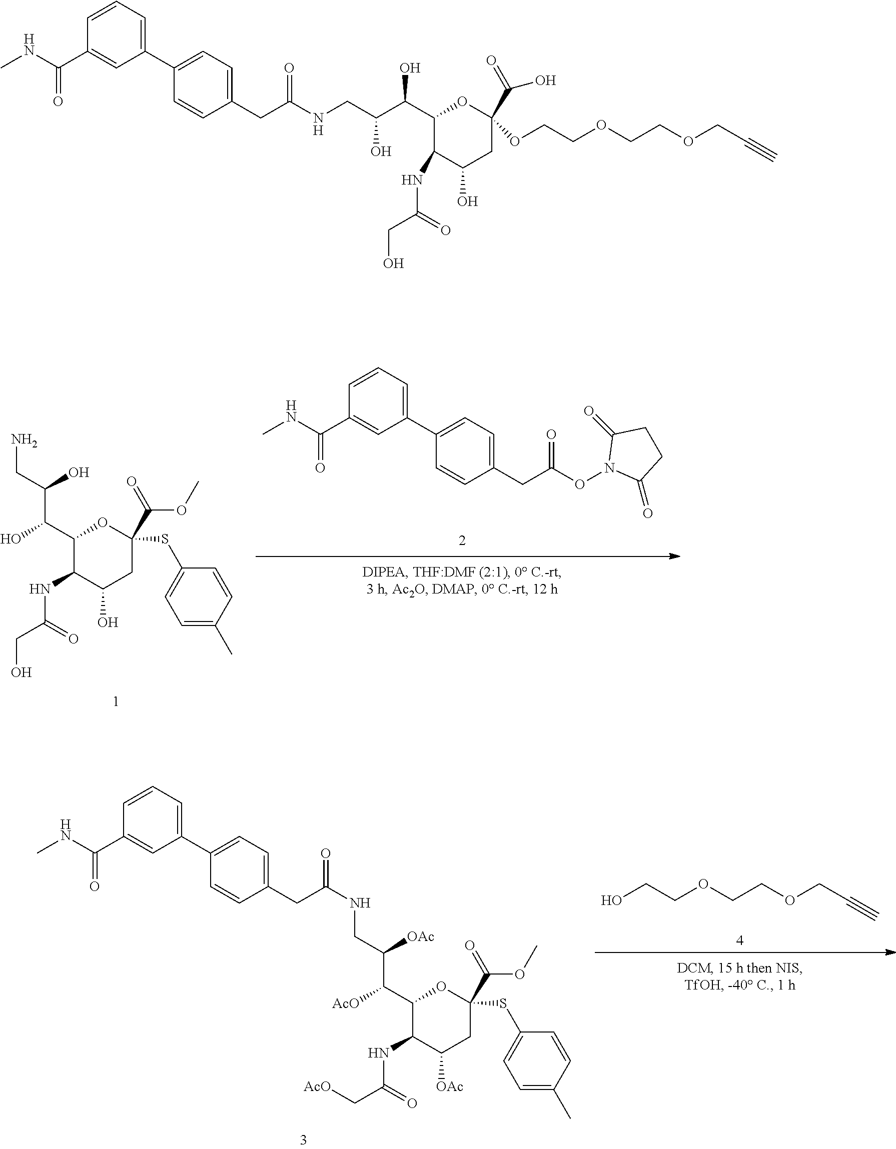

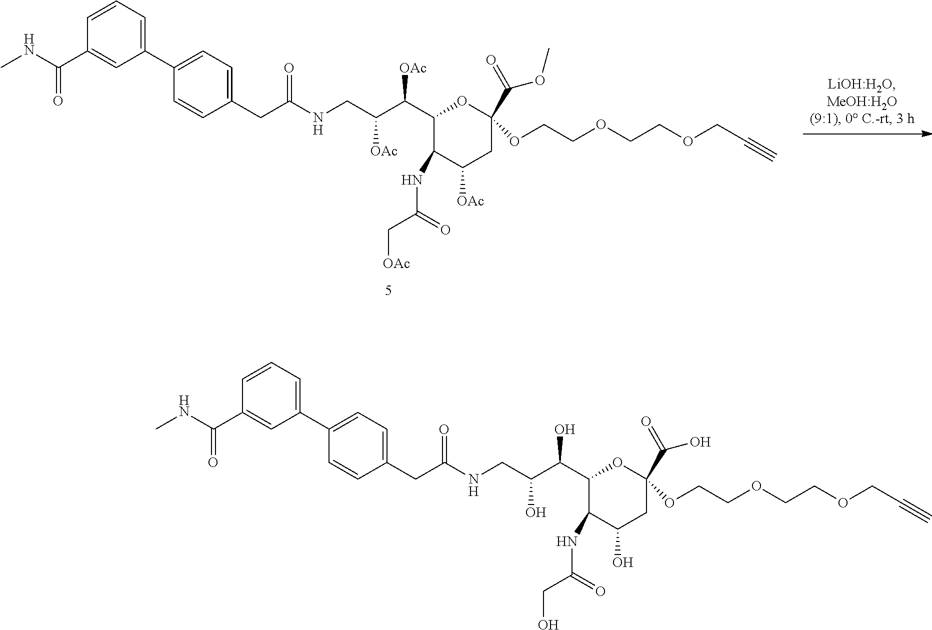

### Synthesis of (1R,2R)-1-((2R,3R,4S,6S)-4-acetoxy-3-(2-acetoxyacetamido)-6-(methoxycarbonyl)-6-(p-tolylthio)tetrahydro-2H-pyran-2-yl)-3-(2-(3′-(methylcarbamoyl)-[1,1′-biphenyl]-4-yl)acetamido)propane-1,2-diyl diacetate (3)

To a stirred solution of methyl (2S,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (1, 1.00 g, 2.250 mmol) and 2,5-dioxopyrrolidin-1-yl 2-(3′-(methylcarbamoyl)-[1,1′-biphenyl]-4-yl)acetate (2, 0.659 g, 1.800 mmol) in tetrahydrofuran (8.0 mL) and N,N-dimethylformamide (4.0 mL), was added ethylbis(propan-2-yl)amine (1.96 mL, 11.20 mmol) at 0° C. then stirred at room temperature for 4 h. Acetic anhydride (2.13 mL, 22.50 mmol) and N, N-dimethyl aniline (0.137 g, 1.12 mmol) was added at 0° C. and the resulting mixture was stirred at room temperature for 12 h. After completion the mixture was diluted with water and ethyl acetate. The organic layer was separated, dried, filtered, concentrated, and the crude residue obtained was purified by silica gel column chromatography using ethyl acetate in hexane as eluents to afford the desired product (3). Yield: 0.60 g, 30.87%; LCMS (ESI) m/z 864.05 [M+H]⁺.

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(3′-(methylcarbamoyl)-[1,1′-biphenyl]-4-yl)acetamido)propane-1,2-diyl diacetate (5)

A mixture of (1R,2R)-1-((2R,3R,4S,6S)-4-acetoxy-3-(2-acetoxyacetamido)-6-(methoxycarbonyl)-6-(p-tolylthio)tetrahydro-2H-pyran-2-yl)-3-(2-(3′-(methylcarbamoyl)-[1,1′-biphenyl]-4-yl)acetamido)propane-1,2-diyl diacetate (3, 0.50 g, 0.579 mmol), 2-(2-(prop-2-yn-1-yloxy)ethoxy)ethan-1-ol (4, 0.417 g, 2.890 mmol) and activated powdered 4 Å molecular sieves (1.00 g, 100% w/w) in anhydrous dichloromethane (10.0 mL) was stirred at room temperature for 15 h. The solution was then cooled to −40° C. followed by addition of 1-iodopyrrolidine-2,5-dione (0.326 g, 1.450 mmol) and trifluoromethanesulfonic acid (0.051 mL, 0.579 mmol). The reaction mixture was stirred at this temperature for another 2 h. After completion, the reaction mixture was quenched by addition of triethyl amine (neutral pH) and warmed up gradually to room temperature. The reaction mixture was filtered, and the filtrate was washed with deionized water. The organic layer was separated, dried, filtered, concentrated, and the crude residue obtained was purified by silica gel column chromatography using ethyl acetate in hexane as eluents to afford the desired product (5). Yield: 0.40 g, 65.16%; LCMS m/z 881.80 [M−H]⁻.

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(3′-(methylcarbamoyl)-[1,1′-biphenyl]-4-yl)acetamido)propane-1,2-diyl diacetate (5, 0.750 g, 0.849 mmol) in methanol (10.0 mL), was added a solution of lithium hydroxide monohydrate (0.214 g, 5.09 mmol) in water (0.5 mL). The reaction mixture was stirred at room temperature for 6 h. After completion, the reaction mixture was treated with acidic resin (Dowex 50H⁺) until pH ˜6 and the suspension was filtered. The filtrate was concentrated, and the crude residue obtained was purified by prep HPLC using acetonitrile in water (+0.1% TFA) as eluents to afford the desired product (Cpd. 80). Yield: 0.019 g, 3.19%; LCMS m/z 702.30 [M+H]⁺; ¹H NMR (400 MHz, methanol-d4) (8.07 (t, J=1.6 Hz, 1H), 7.89 (d, J=8.0 Hz, 1H), 7.78 (t, J=8.0 Hz, 2H), 7.53 (t, J=7.6 Hz, 1H), 7.42 (d, J=8.4 Hz, 2H), 4.16 (d, J=2.4 Hz, 2H), 4.01 (s, 2H), 3.91-3.80 (m, 4H), 3.74-3.56 (m, 11H), 3.38 (dd, J=8.4 & 1.2 Hz, 1H), 3.27-3.23 (m, 1H), 2.94 (s, 3H), 2.83 (t, J=2.4 Hz, 1H), 2.72 (dd, J=12.8 & 4.0 Hz, 1H), 1.74 (t, J=6.8 Hz, 1H).

### (2R,4S,5R,6R)-6-((1R,2R)-1,2-dihydroxy-3-(2-(4-(6-oxo-1,6-dihydropyridin-3-yl)phenyl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 74)

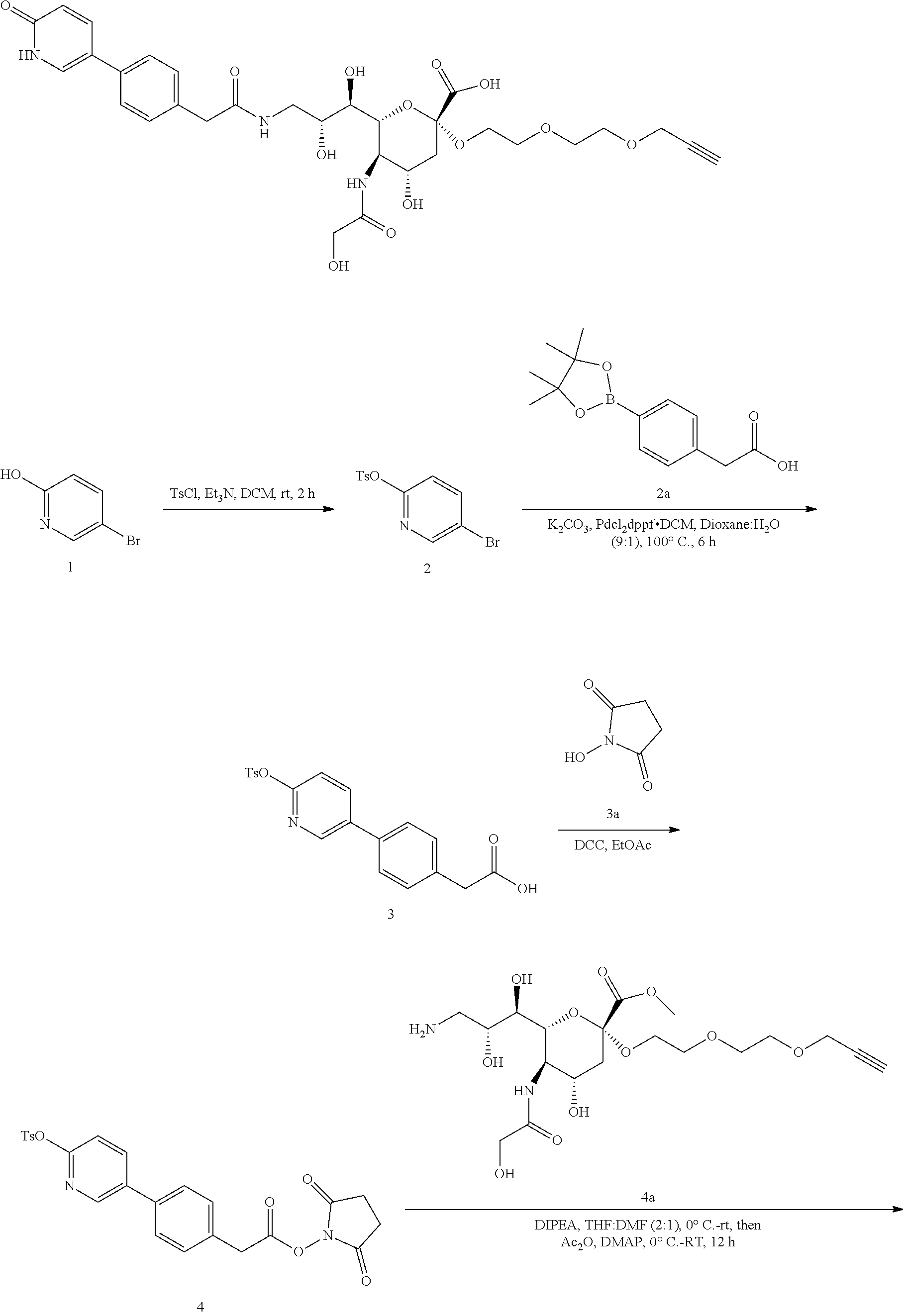

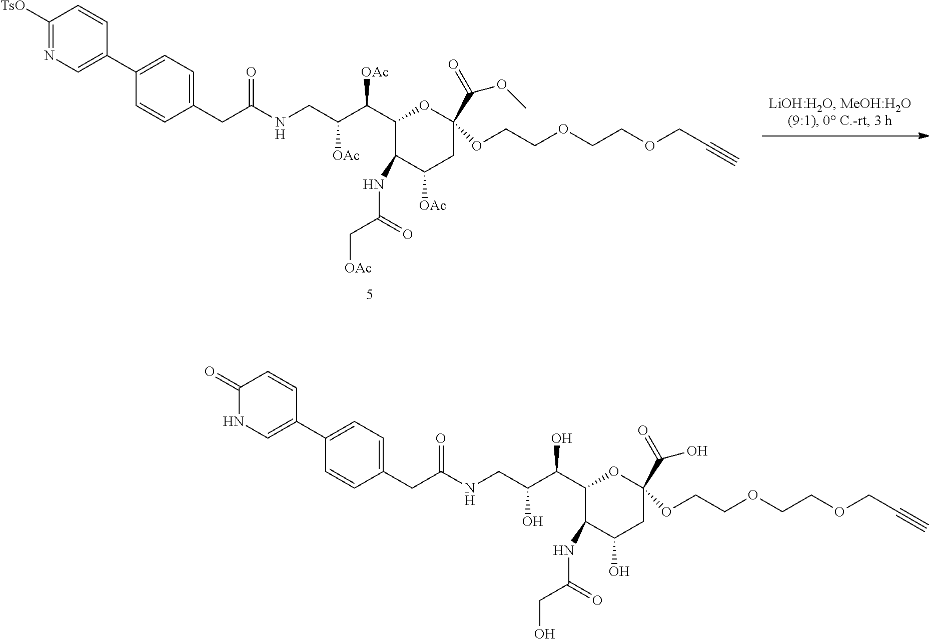 [SMILES: C#CCOCCOCCO[C@]1(C(=O)OC)C[C@H](OC(C)=O)[C@@H](NC(=O)COC(C)=O)[C@H]([C@H](OC(C)=O)[C@@H](CNC(=O)Cc2ccc(-c3ccc(OS(=O)(=O)c4ccc(C)cc4)nc3)cc2)OC(C)=O)O1 ; "LiOH:H2O, MeOH:H2O(9:1), 0° C.-rt, 3 h" ; C#CCOCCOCCO[C@]1(C(=O)O)C[C@H](O)[C@@H](NC(=O)CO)[C@H]([C@H](O)[C@H](O)CNC(=O)Cc2ccc(-c3ccc(=O)[nH]c3)cc2)O1]

### Synthesis of 5-bromopyridin-2-yl 4-methylbenzenesulfonate (2)

To a stirred solution of 5-bromopyridin-2-ol (1, 2.00 g, 11.50 mmol) in dichloromethane (20.0 mL), 4-methylbenzene-1-sulfonyl chloride (2.63 g, 13.8 mmol), triethylamine (1.35 mL, 23.0 mmol) and N,N-dimethylpyridin-4-amine (0.140 g, 115 mmol) was added at 0° C. and then stirred at room temperature for 2 h. After completion the mixture was diluted with water and ethyl acetate. The organic layer was separated, dried, filtered, concentrated, and the crude residue obtained was purified by silica gel column chromatography using ethyl acetate in hexane as eluents to afford the desired product (2). Yield: 2.20 g, 58.32%; LCMS (ESI) m/z 329.80 [M+H]⁺.

### Synthesis of 2-(4-(6-(tosyloxy)pyridin-3-yl)phenyl)acetic acid (3)

A mixture of 5-bromopyridin-2-yl 4-methylbenzenesulfonate (2, 2.0 g, 6.090 mmol), 2-(4-(4,4,5,5-tetramethyl-1,3,2-dioxaborolan-2-yl)phenyl)acetic acid (2a, 1.76 g, 6.700 mmol) and potassium carbonate (2.52 g, 18.270 mmol) in 1,4-dioxane (20.0 mL) and water (2.0 mL). The reaction mixture was degassed by bubbling argon for 10 min, then 1,1-bis(diphenylphosphino)ferrocene dichloropalladium (0.496 g, 0.609 mmol) was added at room temperature and stirred for 6 h at 100° C. After completion the mixture was diluted with water and ethyl acetate. The organic layer was separated, dried, filtered, concentrated, and the crude residue obtained was purified by silica gel column chromatography using ethyl acetate in hexane as eluents to afford the desired product (3). Yield: 1.50 g, 64.19%; LCMS m/z 384.10 [M+H]⁺.

### Synthesis of 2,5-dioxopyrrolidin-1-yl 2-(4-(6-(tosyloxy)pyridin-3-yl)phenyl)acetate (4)

To a stirred solution of 2-(4-(6-(tosyloxy)pyridin-3-yl)phenyl)acetic acid (3, 1.50 g, 3.91 mmol) in ethyl acetate(15.0 mL), 1-hydroxypyrrolidine-2,5-dione (0.675 g, 5.870 mmol) and N,N′-dicyclohexylcarbodiimide (1.21 g, 5.870 mmol) was added at 0° C. and the reaction was stirred at room temperature for 6 h. After completion the mixture was filtered, and the filtrate was washed with ethyl acetate, dried, then concentrated, and the crude residue obtained was purified by silica gel column chromatography using ethyl acetate in hexane as eluents to afford the desired product (4). Yield: 1.50 g, 79.80%; LCMS m/z 480.85 [M+H]⁺.

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(4-(6-(tosyloxy)pyridin-3-yl)phenyl)acetamido)propane-1,2-diyl diacetate (5)

To a stirred solution of methyl (2R,4S,5R,6R)-6-((1R,2R)-3-amino-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylate (4a, 0.50 g, 1.080 mmol) and 2,5-dioxopyrrolidin-1-yl 2-(4-(2-(tosyloxy)pyridin-4-yl)phenyl)acetate (4, 0.414 g, 0.861 mmol) in tetrahydrofuran (4.0 mL) and N,N-dimethylformamide (2.00 mL), was added ethylbis(propan-2-yl)amine (0.93 mL, 5.380 mmol) at 0° C. then stirred at room temperature for 4 h. Acetic anhydride (1.02 mL, 10.800 mmol) and N, N-dimethyl aniline (0.065 g, 0.538 mmol) was added at 0° C. and the resulting mixture was stirred at room temperature for 12 h. After completion the mixture was diluted with water and ethyl acetate. The organic layer was separated, dried, filtered, concentrated, and the crude residue obtained was purified by silica gel column chromatography using ethyl acetate in hexane as eluents to afford the desired product (5). Yield: 0.350 g, 32.58%; LCMS (ESI) m/z 995.70 [M−H]⁻.

### Synthesis of (2R,4S,5R,6R)-6-((1R,2R)-1,2-dihydroxy-3-(2-(4-(6-oxo-1,6-dihydropyridin-3-yl)phenyl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. No. 74)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(4-(6-(tosyloxy)pyridin-3-yl)phenyl)acetamido)propane-1,2-diyl diacetate (5, 0.350 g, 0.350 mmol) in methanol (5.0 mL), was added a solution of lithium hydroxide monohydrate (0.088 g, 2.100 mmol) in water (0.5 mL). The reaction mixture was stirred at room temperature for 6 h. After completion, the reaction mixture was treated with acidic resin (Dowex 50H⁺) until pH ˜6 and the suspension was filtered. The filtrate was concentrated, and the crude residue obtained was purified by prep HPLC using acetonitrile in water (+0.1% TFA) as eluents to afford the desired product (Cpd. 74). Yield: 0.006 g, 2.59%; LCMS m/z 662.38 [M+H]⁺; ¹H NMR (400 MHz, methanol-d4) δ 7.96 (dd, J=9.6 & 2.4 Hz, 1H), 7.69 (d, J=2.4 Hz, 1H), 7.48 (d, J=8.4 Hz, 2H), 7.37 (d, J=8.0 Hz, 2H), 6.64 (d, J=9.2 Hz, 1H), 4.19-4.17 (m, 3H), 4.00 (s, 2H), 3.90-3.86 (m, 2H), 3.86-3.83 (m, 1H), 3.83-3.76 (m, 2H), 3.71-3.68 (m, 3H), 3.66-3.63 (m, 4H), 3.62-3.57 (m, 5H), 3.37 (s, 1H), 3.26-3.21 (m, 2H), 2.83 (t, J=2.4 Hz, 2H), 2.76 (dd, J=11.6 & 3.2 Hz, 1H), 1.70 (t, J=11.2 hz, 1H).

### (2R,3S,4R,5R,6R)-6-((1R,2R)-3-(2-([1,1′-biphenyl]-4-yl)acetamido)-1,2-dihydroxypropyl)-3-hydroxy-5-(2-hydroxyacetamido)-2-(2-(2-(prop-2-yn-1-yloxy)ethoxy)ethoxy)-4-propionamidotetrahydro-2H-pyran-2-carboxylic acid (Compound 178)

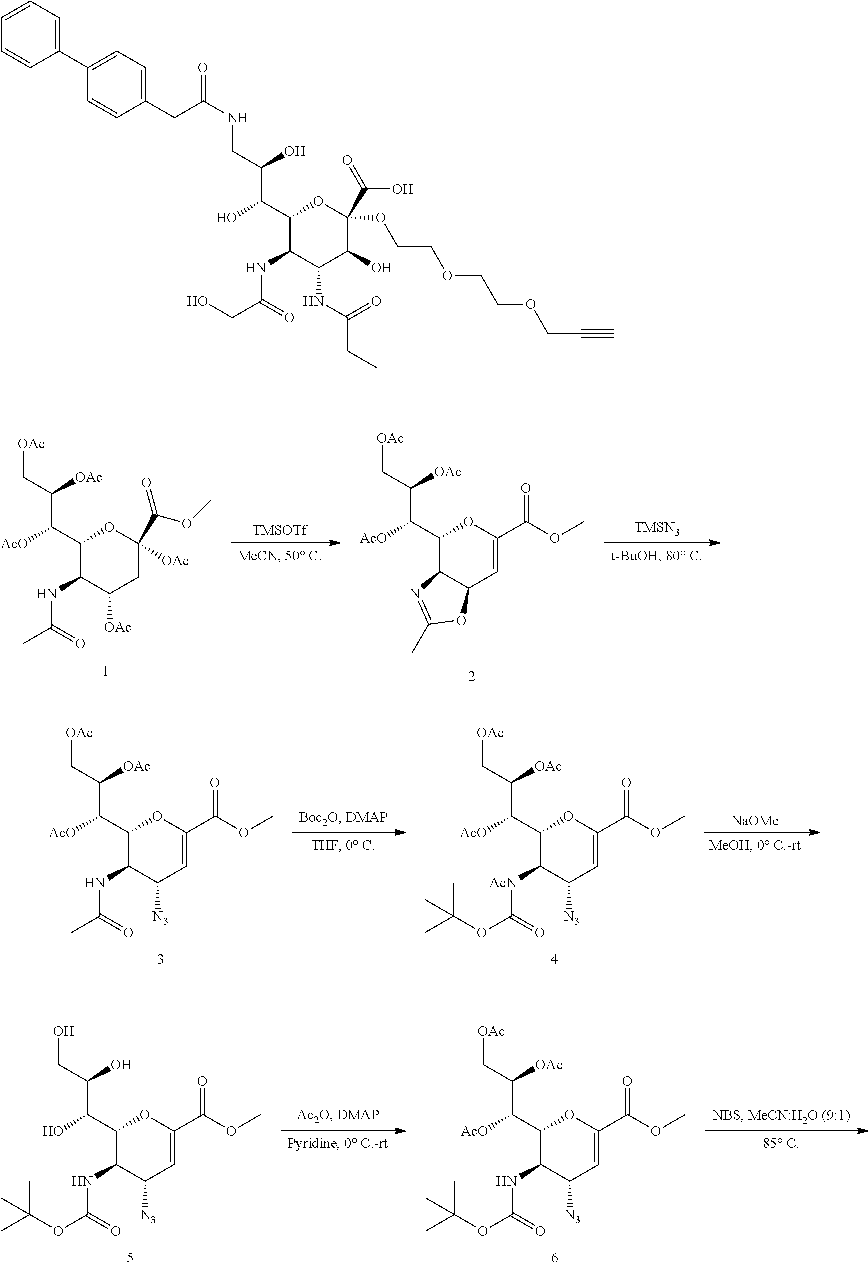

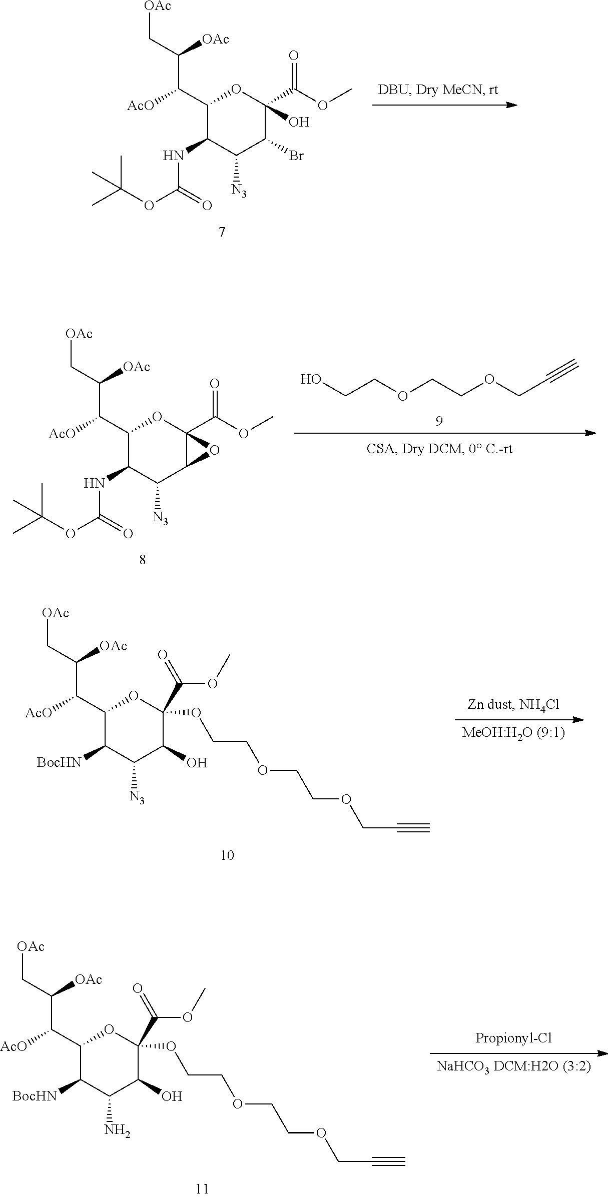

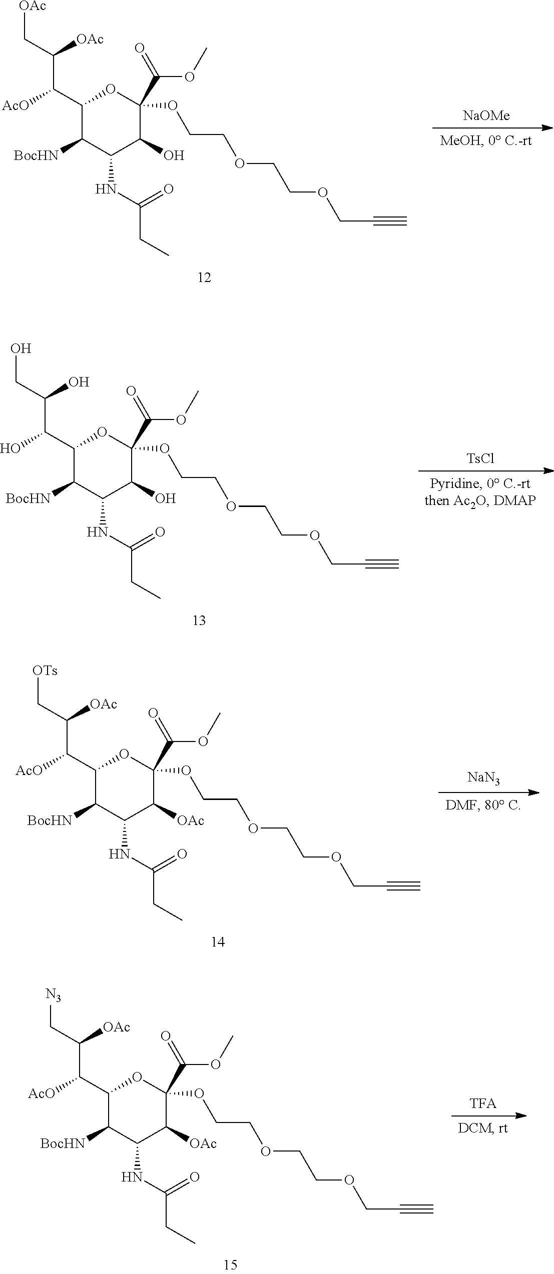

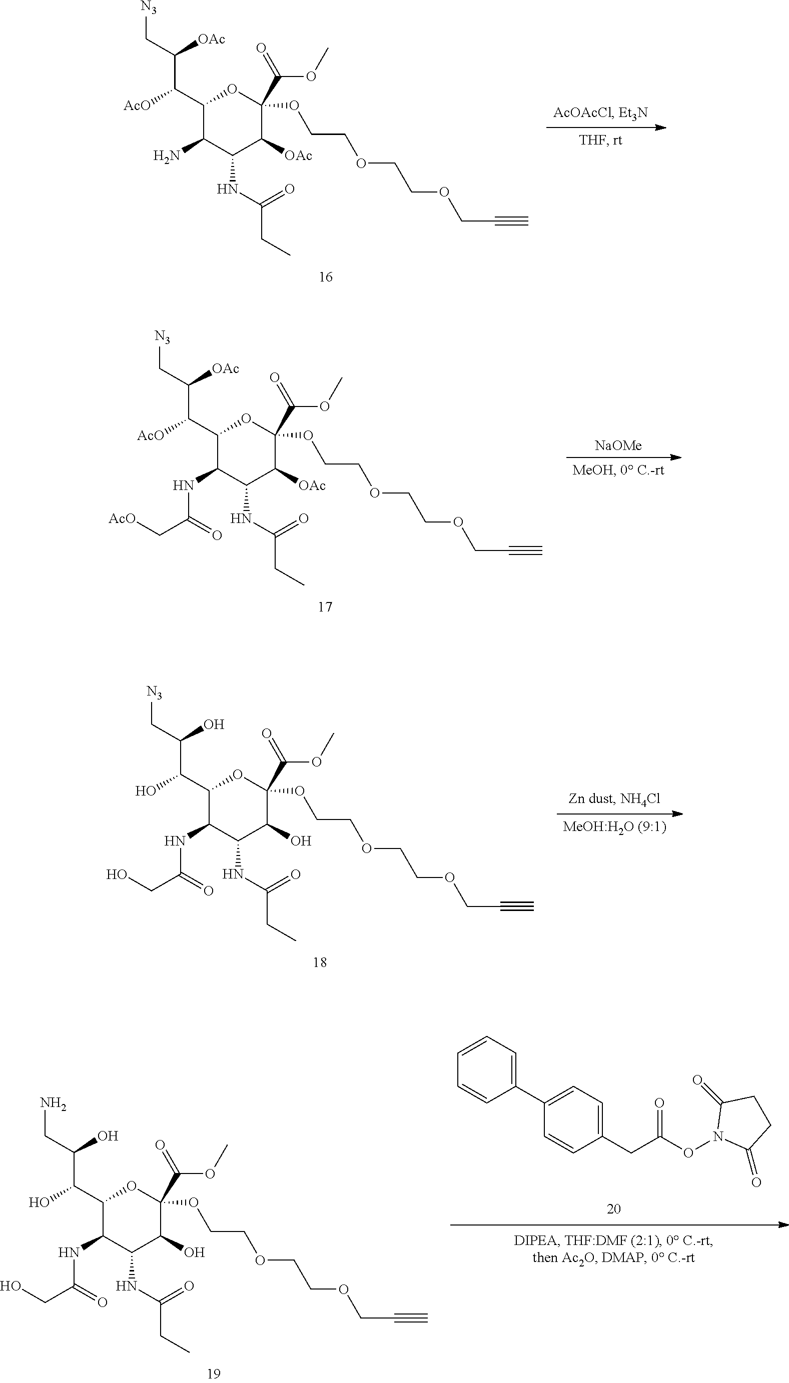

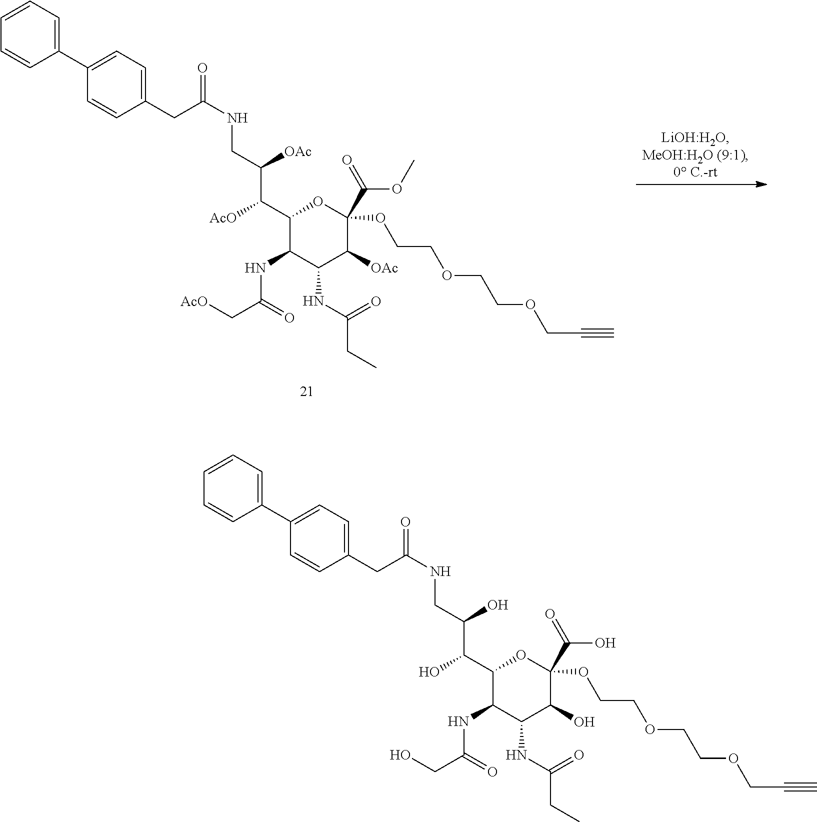 [SMILES: C#CCOCCOCCO[C@@]1(C(=O)OC)O[C@@H]([C@H](OC(C)=O)[C@@H](CNC(=O)Cc2ccc(-c3ccccc3)cc2)OC(C)=O)[C@H](NC(=O)COC(C)=O)[C@@H](NC(=O)CC)[C@@H]1OC(C)=O ; "LiOH:H2O,MeOH:H2O (9:1),0° C.-rt" ; C#CCOCCOCCO[C@@]1(C(=O)O)O[C@@H]([C@H](O)[C@H](O)CNC(=O)Cc2ccc(-c3ccccc3)cc2)[C@H](NC(=O)CO)[C@@H](NC(=O)CC)[C@@H]1O]

### Synthesis of (1S,2R)-1-((3aR,4R,7aR)-6-(methoxycarbonyl)-2-methyl-3a,7a-dihydro-4H-pyrano[3,4-d]oxazol-4-yl)propane-1,2,3-triyl triacetate (2)

To a stirred solution of methyl (2S,4S,5R,6R)-2,4-bis(acetyloxy)-5-acetamido-6-[(1S,2R)-1,2,3-tris(acetyloxy)propyl]oxane-2-carboxylate (1, 20.0 g, 1.0 eq, 37.5 mmol) in dry Acetonitrile (200.0 mL) at 0° C. was added trimethylsilyl trifluoromethanesulfonate (16.9 ml, 93.7 mmol) drop-wise. The mixture was stirred at 50° C. for 4 h then quenched by addition of triethyl amine until pH 7. The reaction mixture concentrated was concentrated to afford the desired product (2) as a crude (Crude wt: 20 g) which was used for the next step without further purification.

### Synthesis of (1S,2R)-1-((2R,3R,4S)-3-acetamido-4-azido-6-(methoxycarbonyl)-3,4-dihydro-2H-pyran-2-yl)propane-1,2,3-triyl triacetate (3)

To a stirred solution of methyl (3aR,4R,7aR)-2-methyl-4-[(1S,2R)-1,2,3-tris(acetyloxy)propyl]-3aH,4H,7aH-pyrano[3,4-d][1,3]oxazole-6-carboxylate (2, 20.0 g, 48.4 mmol) in tert-butanol (200 mL), was added azidotrimethylsilane (44.5 mL, 339.8 mmol) dropwise at room temperature. The reaction mixture was stirred at 80° C. for 10 h, then cooled to room temperature and concentrated, and the crude residue obtained was purified by silica gel column chromatography using ethyl acetate in heptane as eluents to afford the desired product (3). Yield: 12.1 g; 41.1%). LCMS m/z 457.17 [M+H]⁺.

### Synthesis of (1S,2R)-1-((2R,3R,4S)-4-azido-3-(N-(tert-butoxycarbonyl)acetamido)-6-(methoxycarbonyl)-3,4-dihydro-2H-pyran-2-yl)propane-1,2,3-triyl triacetate (4)

(1S,2R)-1-((2R,3R,4S)-3-acetamido-4-azido-6-(methoxycarbonyl)-3,4-dihydro-2H-pyran-2-yl)propane-1,2,3-triyl triacetate (3, 2.8 g, 6.13 mmol) was stirred in tetrahydrofuran (30 mL), 4 Å molecular sieves was added and the mixture was stirred for 1 h at 0° C. Di-tert-butyl dicarbonate (7 mL; 30.7 mmol) and 4-Dimethylaminopyridine (1.5 g, 12.3 mmol) were added was stirred for 5 min at room temperature then 12 h at 65° C. The reaction mixture was filtered, and the filtrate was concentrated. The residue was dissolved in ethyl acetate and the organic layer was washed with sat. sodium chloride, dried, filtered, and the filtrate was concentrated, and the crude residue obtained was purified by silica gel column chromatography using ethyl acetate in hexane as eluents to afford the desired product (4). Yield: 1.6 g, 46.86%; LCMS m/z 574.15 [M+H3O]⁺.

### Synthesis of methyl (2R,3R,4S)-4-azido-3-((tert-butoxycarbonyl)amino)-2-((1R,2R)-1,2,3-trihydroxypropyl)-3,4-dihydro-2H-pyran-6-carboxylate (5)

To a mixture of (1S,2R)-1-((2R,3R,4S)-4-azido-3-(N-(tert-butoxycarbonyl)acetamido)-6-(methoxycarbonyl)-3,4-dihydro-2H-pyran-2-yl)propane-1,2,3-triyl triacetate (4, 1.6 g; 2.87 mmol) in methanol (16 mL) was added 25% sodium methoxide in methanol (0.062 mL, 0.287 mmol) drop-wise (pH should not be more than 9). The reaction mixture was stirred for 30 min at room temperature then neutralized with acidic resin (Dowex-Hydrogen form), filtered, and the filtrate was concentrated to afford the desired product (5) as a crude which was used for the following step without further purification. Yield: 1.1 g, Crude; LCMS m/z 406.15 [M+H3O]⁺.

### Synthesis of (1S,2R)-1-((2R,3R,4S)-4-azido-3-((tert-butoxycarbonyl)amino)-6-(methoxycarbonyl)-3,4-dihydro-2H-pyran-2-yl)propane-1,2,3-triyl triacetate (6)

To a solution of methyl (2R,3R,4S)-4-azido-3-((tert-butoxycarbonyl)amino)-2-((1R,2R)-1,2,3-trihydroxypropyl)-3,4-dihydro-2H-pyran-6-carboxylate (5, 4 g, 10.3 mmol) in pyridine (10 mL), was added acetic anhydride (5.84 mL, 61.8 mmol) at 0° C. The mixture was stirred for 10 min at the same temperature after which ice-bath was removed and the mixture was stirred for 16 h at room temperature. After completion, the reaction mixture was concentrated. The residue was dissolved in ethyl acetate and the organic layer was washed with 1 N hydrochloric acid solution. The qqueous layer was then extracted twice with ethyl acetate. The combined organic layers were washed with sat. sodium bicarbonate, water, brine, then dried, filtered, and concentrated, and the crude residue was purified by silica gel column chromatography using ethyl acetate in heptane as eluents to afford the desired product (6). Yield: 4.1 g, 77.38%; LCMS m/z 516.03 [M+H]⁺.

### Synthesis of (1S,2R)-1-((2R,3R,4R,5R,6R)-4-azido-5-bromo-3-((tert-butoxycarbonyl)amino)-6-hydroxy-6-(methoxycarbonyl)tetrahydro-2H-pyran-2-yl)propane-1,2,3-triyl triacetate (7)

A solution of (1S,2R)-1-((2R,3R,4S)-4-azido-3-((tert-butoxycarbonyl)amino)-6-(methoxycarbonyl)-3,4-dihydro-2H-pyran-2-yl)propane-1,2,3-triyl triacetate (6, 3.00 g, 5.83 mmol) in acetonitrile:water (9:1, 50 mL) was stirred at 80° C. for 1 h then cooled down to room temperature. N-bromosuccinimide (1.78 g, 29.2 mmol) was added was then stirred for 5 h at room temperature. After completion, the reaction mixture was concentrated, and co-evaporated with acetonitrile (3-4 times). The crude residue obtained was purified by silica gel column chromatography using ethyl acetate in hexane as eluents to afford the desired product (7). Yield: 2.5 g, 70.12%; ELSD-MS m/z 612.1 [M+H]⁺.

### Synthesis of (1S,2R)-1-((1S,3R,4R,5R,6S)-5-azido-4-((tert-butoxycarbonyl)amino)-1-(methoxycarbonyl)-2,7-dioxabicyclo[4.1.0]heptan-3-yl)propane-1,2,3-triyl triacetate (8)

To a solution of (1S,2R)-1-((2R,3R,4R,5R,6R)-4-azido-5-bromo-3-((tert-butoxycarbonyl)amino)-6-hydroxy-6-(methoxycarbonyl)tetrahydro-2H-pyran-2-yl)propane-1,2,3-triyl triacetate (7, 5.00 g, 8.18 mmol) in dry acetonitrile (30.0 mL), was added 1,8-Diazabicyclo[5.4.0]undec-7-ene (0.122 mL, 0.818 mmol) dropwise at room temperature. The reaction mixture was stirred at room temperature for 30 min. then concentrated to ⅔ of its volume and directly loaded over silica gel chromatography. The product was purified using ethyl acetate in heptane as eluents to afford the desired product (8). Yield: 1.5 g, 34.58%; ELSD-MS m/z 531.1 [M+H]⁺.

### Synthesis of (1S,2R)-1-((2R,3R,4R,5S,6R)-4-azido-3-((tert-butoxycarbonyl)amino)-5-hydroxy-6-(methoxycarbonyl)-6-(2-(2-(prop-2-yn-1-yloxy)ethoxy)ethoxy)tetrahydro-2H-pyran-2-yl)propane-1,2,3-triyl triacetate (10)

To a mixture of (1S,2R)-1-((1S,3R,4R,5R,6S)-5-azido-4-((tert-butoxycarbonyl)amino)-1-(methoxycarbonyl)-2,7-dioxabicyclo[4.1.0]heptan-3-yl)propane-1,2,3-triyl triacetate (8, 1.93 g, 3.64 mmol) in dry dichloromethan (20 mL) with 4 Å molecular sieves (2.0 g), 2-[2-(prop-2-yn-1-yloxy)ethoxy]ethan-1-ol (9, 5.25 g, 36.4 mmol) was added camphor sulphonic acid (1.93 g, 8.31 mmol) at room temperature. The reaction mixture was stirred at room temperature for 5 h. After completion, the reaction mixture was quenched with saturated sodium bicarbonate solution and extracted with dichloromethane. The organic layer was dried, filtered, and the filtrate was concentrated, and the crude residue was purified by silica gel column chromatography using ethyl acetate in hexane as eluents to afford the desired product (10). Yield: 1.5 g, 61.11%; ELSD-MS m/z 692.15 [M+H3O]⁺.

### Synthesis of (1S,2R)-1-((2R,3R,4R,5S,6R)-4-amino-3-((tert-butoxycarbonyl)amino)-5-hydroxy-6-(methoxycarbonyl)-6-(2-(2-(prop-2-yn-1-yloxy)ethoxy)ethoxy)tetrahydro-2H-pyran-2-yl)propane-1,2,3-triyl triacetate (11)

To a mixture of (1S,2R)-1-((1S,3R,4R,5R,6S)-5-azido-4-((tert-butoxycarbonyl)amino)-1-(methoxycarbonyl)-2,7-dioxabicyclo[4.1.0]heptan-3-yl)propane-1,2,3-triyl triacetate (10, 3 g, 4.45 mmol) in methanol:water (9:1, 30 mL), were successively added zinc dust (2.91 g, 44.5 mmol) and ammonium chloride (2.38 g, 44.5 mmol) at 0° C. then stirred at room temperature for 1 h. After completion, the reaction mixture was filtered over celite, and the filtrate was concentrated to afford the desired product (11) as a crude which was used for the next reaction without further purification. Yield: 3 g, Crude; ELSD-MS m/z 649.10 [M+H]⁺.

### Synthesis of (1S,2R)-1-((2R,3R,4R,5S,6R)-3-((tert-butoxycarbonyl)amino)-5-hydroxy-6-(methoxycarbonyl)-6-(2-(2-(prop-2-yn-1-yloxy)ethoxy)ethoxy)-4-propionamidotetrahydro-2H-pyran-2-yl)propane-1,2,3-triyl triacetate (12)

The crude (1S,2R)-1-((2R,3R,4R,5S,6R)-4-amino-3-((tert-butoxycarbonyl)amino)-5-hydroxy-6-(methoxycarbonyl)-6-(2-(2-(prop-2-yn-1-yloxy)ethoxy)ethoxy)tetrahydro-2H-pyran-2-yl)propane-1,2,3-triyl triacetate (11, 3.0 g, 4.62 mmol) was dissolved in CHCl₃/H₂O (30 mL:20 mL) and cooled to 0° C. Solid sodium bicarbonate (1.55 g, 18.5 mmol) and propanoyl chloride (1.21 mL, 13.9 mmol) were sequentially added to the reaction mixture at 0° C. The reaction mixture was stirred for 4 h then diluted with chloroform and washed with water. The aqueous layer was extracted twice with dichloromethane. The combined organic layers were washed with brine, dried, filtered, and the filtrate was concentrated. The crude product obtained was purified by silica gel column chromatography using methanol in dichloromethane as eluents to afford the desired product (12). Yield: 2.5 g, 76.7%; LCMS m/z 705.55 [M+H]⁺.

### Synthesis of methyl (2R,3S,4R,5R,6R)-5-((tert-butoxycarbonyl)amino)-3-hydroxy-2-(2-(2-(prop-2-yn-1-yloxy)ethoxy)ethoxy)-4-propionamido-6-((1R,2R)-1,2,3-trihydroxypropyl)tetrahydro-2H-pyran-2-carboxylate (13)

(1S,2R)-1-((2R,3R,4R,5S,6R)-3-((tert-butoxycarbonyl)amino)-5-hydroxy-6-(methoxycarbonyl)-6-(2-(2-(prop-2-yn-1-yloxy)ethoxy)ethoxy)-4-propionamidotetrahydro-2H-pyran-2-yl)propane-1,2,3-triyl triacetate (12, 1.5 g, 2.13 mmol) was dissolved in methanol (20 mL) and 25% sodium methoxide in methanol (0.408 mL, 1.89 mmol) was added. The reaction mixture was stirred for 30 min at room temperature then neutralized with acidic resin (Dowex-Hydrogen form) until pH˜6, filtered, and the filtrate was concentrated, and the crude residue obtained was triturated with diethyl ether and dried under vacuum to afford the desired product (13) as a crude which was used for next step without further purification. Yield: 1.2 g, Crude; ELSD-MS m/z 579.25 [M+H]⁺.

### Synthesis of (1S,2R)-1-((2R,3R,4R,5S,6R)-5-acetoxy-3-((tert-butoxycarbonyl)amino)-6-(methoxycarbonyl)-6-(2-(2-(prop-2-yn-1-yloxy)ethoxy)ethoxy)-4-propionamidotetrahydro-2H-pyran-2-yl)-3-(tosyloxy)propane-1,2-diyl diacetate (14)

To a solution of methyl (2R,3S,4R,5R,6R)-5-((tert-butoxycarbonyl)amino)-3-hydroxy-2-(2-(2-(prop-2-yn-1-yloxy)ethoxy)ethoxy)-4-propionamido-6-((1R,2R)-1,2,3-trihydroxypropyl)tetrahydro-2H-pyran-2-carboxylate (13, 0.7 g, 1.21 mmol) in pyridine (10 mL) at 0° C., were sequentially added 4-methylbenzene-1-sulfonyl chloride (0.507 g, 2.66 mmol) and a catalytic amount of N,N-dimethyl-4-amino-pyridine (0.014 g, 0.121 mmol) The reaction mixture was stirred overnight from 0° C. to room temperature. After complete consumption of the starting material, acetic anhydride (1 mL) was added dropwise and the mixture was allowed to warm up to room temperature overnight. The reaction mixture was concentrated, and the crude residue obtained was purified by silica gel column chromatography using MeOH in DCM to afford the desired product (14). Yield: 0.450 g, 43.31%; ELSD-MS m/z 859.00 [M+H]⁺.

### Synthesis of (1R,2R)-1-((2R,3R,4R,5S,6R)-5-acetoxy-3-((tert-butoxycarbonyl)amino)-6-(methoxycarbonyl)-6-(2-(2-(prop-2-yn-1-yloxy)ethoxy)ethoxy)-4-propionamidotetrahydro-2H-pyran-2-yl)-3-azidopropane-1,2-diyl diacetate (15)

To a solution of methyl (2R,3S,4R,5R,6R)-3-(acetyloxy)-6-[(1S,2R)-1,2-bis(acetyloxy)-3-[(4-methylbenzenesulfonyl)oxy]propyl]-5-{[(tert-butoxy)carbonyl]amino}-2-{2-[2-(prop-2-yn-1-yloxy)ethoxy]ethoxy}-4-propanamidooxane-2-carboxylate (14, 0.4 g, 0.466 mmol) in DMF (5 mL), was added sodium azide (0.303 g, 4.66 mmol) was stirred at 80° C. for 12 h. After completion, the reaction mixture was evaporated to dryness, diluted with aq. saturated sodium bicarbonate and extracted with dichloromethane. The organic layer was dried, filtered, and the filtrate was concentrated, and the crude residue obtained was purified by silica gel column chromatography using methanol in dichloromethane to afford the desired product (15). Yield: 0.150 g, 44.14%; ELSD-MS m/z 730.10 [M+H]+

### Synthesis of (1R,2R)-1-((2R,3R,4R,5S,6R)-5-acetoxy-3-amino-6-(methoxycarbonyl)-6-(2-(2-(prop-2-yn-1-yloxy)ethoxy)ethoxy)-4-propionamidotetrahydro-2H-pyran-2-yl)-3-azidopropane-1,2-diyl diacetate (16)

To a solution of (1R,2R)-1-((2R,3R,4R,5S,6R)-5-acetoxy-3-((tert-butoxycarbonyl)amino)-6-(methoxycarbonyl)-6-(2-(2-(prop-2-yn-1-yloxy)ethoxy)ethoxy)-4-propionamidotetrahydro-2H-pyran-2-yl)-3-azidopropane-1,2-diyl diacetate (15, 0.15 g, 0.21 mmol) in dichloromethane (5 mL) was added trifluoroacetic anhydride (0.001 mL, 0.02 mmol) dropwise at 0° C. The reaction mixture was stirred at room temperature for 3 h then concentrated and azeotropped with toluene (3-4 times). The residue obtained was dried under high vacuum to afford the desired product (16) as a crude which was used for next step without further purification. Yield: 0.150 g, Crude; ELSD-MS m/z 630.10 [M+H]⁺.

### Synthesis of (1R,2R)-1-((2R,3R,4R,5S,6R)-5-acetoxy-3-(2-acetoxyacetamido)-6-(methoxycarbonyl)-6-(2-(2-(prop-2-yn-1-yloxy)ethoxy)ethoxy)-4-propionamidotetrahydro-2H-pyran-2-yl)-3-azidopropane-1,2-diyl diacetate (17)

To a solution of crude (1R,2R)-1-((2R,3R,4R,5S,6R)-5-acetoxy-3-(2-acetoxyacetamido)-6-(methoxycarbonyl)-6-(2-(2-(prop-2-yn-1-yloxy)ethoxy)ethoxy)-4-propionamidotetrahydro-2H-pyran-2-yl)-3-azidopropane-1,2-diyl diacetate (16, 0.35 g, 0.56 mmol) in tetrahydrofuran (6 mL), triethylamine (0.39 mL, 0.002 mmol) was added dropwise at 0° C. After 10 min, 2-chloro-2-oxoethyl acetate (0.12 ml, 0.001 mmol) was added was stirred at room temperature for 1 h. After completion, the reaction mixture was quenched by addition of brine and extracted with dichloromethane. The organic layer was dried, filtered, and concentrated, and the crude residue was purified by silica gel column chromatography using ethyl acetate in hexane as eluents to afford the desired product (17). Yield: 0.2 g, 49.31%; ELSD-MS m/z 730.7 [M+H]⁺.

### Synthesis of methyl (2R,3S,4R,5R,6R)-6-((1R,2R)-3-azido-1,2-dihydroxypropyl)-3-hydroxy-5-(2-hydroxyacetamido)-2-(2-(2-(prop-2-yn-1-yloxy)ethoxy)ethoxy)-4-propionamidotetrahydro-2H-pyran-2-carboxylate (18)

To a solution of (1R,2R)-1-((2R,3R,4R,5S,6R)-5-acetoxy-3-(2-acetoxyacetamido)-6-(methoxycarbonyl)-6-(2-(2-(prop-2-yn-1-yloxy)ethoxy)ethoxy)-4-propionamidotetrahydro-2H-pyran-2-yl)-3-azidopropane-1,2-diyl diacetate (17, 0.390 g, 0.534 mmol) in methanol (5 mL) was added 25% sodium methoxide in methanol (0.011 mL, 0.053 mmol) dropwise. The reaction mixture was stirred for 30 min at room temperature then neutralized with acidic resin (Dowex-Hydrogen form) until pH˜6. The suspension was filtered, and the filtrate was concentrated to afford the desired product (18) was as a crude which was used for next step without further purification. Yield: 0.3 g, Crude; ELSD-MS m/z 562.10 [M+H]⁺.

### Synthesis of methyl (2R,3S,4R,5R,6R)-6-((1R,2R)-3-amino-1,2-dihydroxypropyl)-3-hydroxy-5-(2-hydroxyacetamido)-2-(2-(2-(prop-2-yn-1-yloxy)ethoxy)ethoxy)-4-propionamidotetrahydro-2H-pyran-2-carboxylate (19)

To a solution of methyl (2R,3S,4R,5R,6R)-6-((1R,2R)-3-azido-1,2-dihydroxypropyl)-3-hydroxy-5-(2-hydroxyacetamido)-2-(2-(2-(prop-2-yn-1-yloxy)ethoxy)ethoxy)-4-propionamidotetrahydro-2H-pyran-2-carboxylate (18, 0.150 g, 0.267 mmol) in methanol:water (9:1, 5 mL), were successively added zinc dust (0.175 g, 2.67 mmol) and ammonium chloride (0.143 g, 2.67 mmol) at 0° C. The reaction mixture was stirred at room temperature for 1 h then filtered through celite. The filtrate was concentrated to afford the desired product (19) as a crude which was used for the next reaction without further purification. Yield: 0.2 g, Crude; ELSD-MS m/z 536.10 [M+H]⁺.

### Synthesis of methyl (2R,3S,4R,5R,6R)-6-((1R,2R)-3-(2-([1,1′-biphenyl]-4-yl)acetamido)-1,2-dihydroxypropyl)-3-hydroxy-5-(2-hydroxyacetamido)-2-(2-(2-(prop-2-yn-1-yloxy)ethoxy)ethoxy)-4-propionamidotetrahydro-2H-pyran-2-carboxylate (21)

To a solution of methyl (2R,3S,4R,5R,6R)-6-((1R,2R)-3-amino-1,2-dihydroxypropyl)-3-hydroxy-5-(2-hydroxyacetamido)-2-(2-(2-(prop-2-yn-1-yloxy)ethoxy)ethoxy)-4-propionamidotetrahydro-2H-pyran-2-carboxylate (19, 0.1 g, 0.187 mmol) and 2,5-dioxopyrrolidin-1-yl 2-{[1,1′-biphenyl]-4-yl}acetate 2,5-dioxopyrrolidin-1-yl 2-([1,1′-biphenyl]-4-yl)acetate (20, 0.058 g, 0.187 mmol) in tetrahydrofuran (1 mL), was added ethylbis(propan-2-yl)amine (0.163 mL, 0.934 mmol) was stirred at room temperature for 12 h. After completion, acetic anhydride (0.141 mL, 1.49 mmol) and N,N-dimethylpyridin-4-amine (0.002 g, 0.018 mmol) were sequentially added was stirred for 12 h at room temperature. After completion, the reaction mixture was concentrated, and the crude residue obtained was purified by silica gel column chromatography using methanol in dichloromethane as eluents to afford the desired product (21). Yield: 0.150 g, 28.63%; LCMS m/z 898.05 [M+H3O]⁺.

### Synthesis of (2R,3S,4R,5R,6R)-6-((1R,2R)-3-(2-([1,1′-biphenyl]-4-yl)acetamido)-1,2-dihydroxypropyl)-3-hydroxy-5-(2-hydroxyacetamido)-2-(2-(2-(prop-2-yn-1-yloxy)ethoxy)ethoxy)-4-propionamidotetrahydro-2H-pyran-2-carboxylic acid (Cpd. 178)

To a solution of methyl (2R,3S,4R,5R,6R)-6-((1R,2R)-3-(2-([1,1′-biphenyl]-4-yl)acetamido)-1,2-dihydroxypropyl)-3-hydroxy-5-(2-hydroxyacetamido)-2-(2-(2-(prop-2-yn-1-yloxy)ethoxy)ethoxy)-4-propionamidotetrahydro-2H-pyran-2-carboxylate (21, 0.150 g, 0.17 mmol) in methanol (10.0 mL), was added a solution of lithium hydroxide monohydrate (0.04 g, 1.00 mmol) in water (0.5 mL). The reaction mixture was stirred at room temperature for 6 h. After completion, the reaction mixture was treated with acidic resin (Dowex 50H⁺) until pH ˜6 and the suspension was filtered. The filtrate was concentrated, and the crude residue obtained was purified by prep HPLC using acetonitrile in water (+0.1% TFA) as eluents to afford the desired product (Cpd. 178). Yield: 0.011 g, 9.20%; LCMS m/z 716.40 [M+H]⁺; ¹H NMR (400 MHz, Methanol-d₄) δ 7.61-7.56 (m, 4H), 7.44-7.29 (m, 5H), 4.34-4.29 (m, 1H), 4.27-4.23 (m, 2H), 4.17 (d, J=2.4 Hz, 2H), 4.01-3.98 (m, 1H), 3.93 (s, 2H), 3.87-3.83 (m, 1H), 3.78 (d, J=8.0 Hz, 1H), 3.69-3.61 (m, 7H), 3.59 (s, 2H), 3.31-3.21 (m, 2H), 2.84 (t, J=2.4 Hz, 2.4 Hz, 1H), 2.25-2.19 (m, 2H), 1.12 (t, J=7.6 Hz, 7.6 Hz, 3H).

### (2R,3S,4R,5R,6R)-6-((1R,2R)-3-(2-([1,1′-biphenyl]-4-yl)acetamido)-1,2-dihydroxypropyl)-3-hydroxy-5-(2-hydroxyacetamido)-4-(3-methoxybenzamido)-2-(2-(2-(prop-2-yn-1-yloxy)ethoxy)ethoxy)tetrahydro-2H-pyran-2-carboxylic acid (Compound 36)

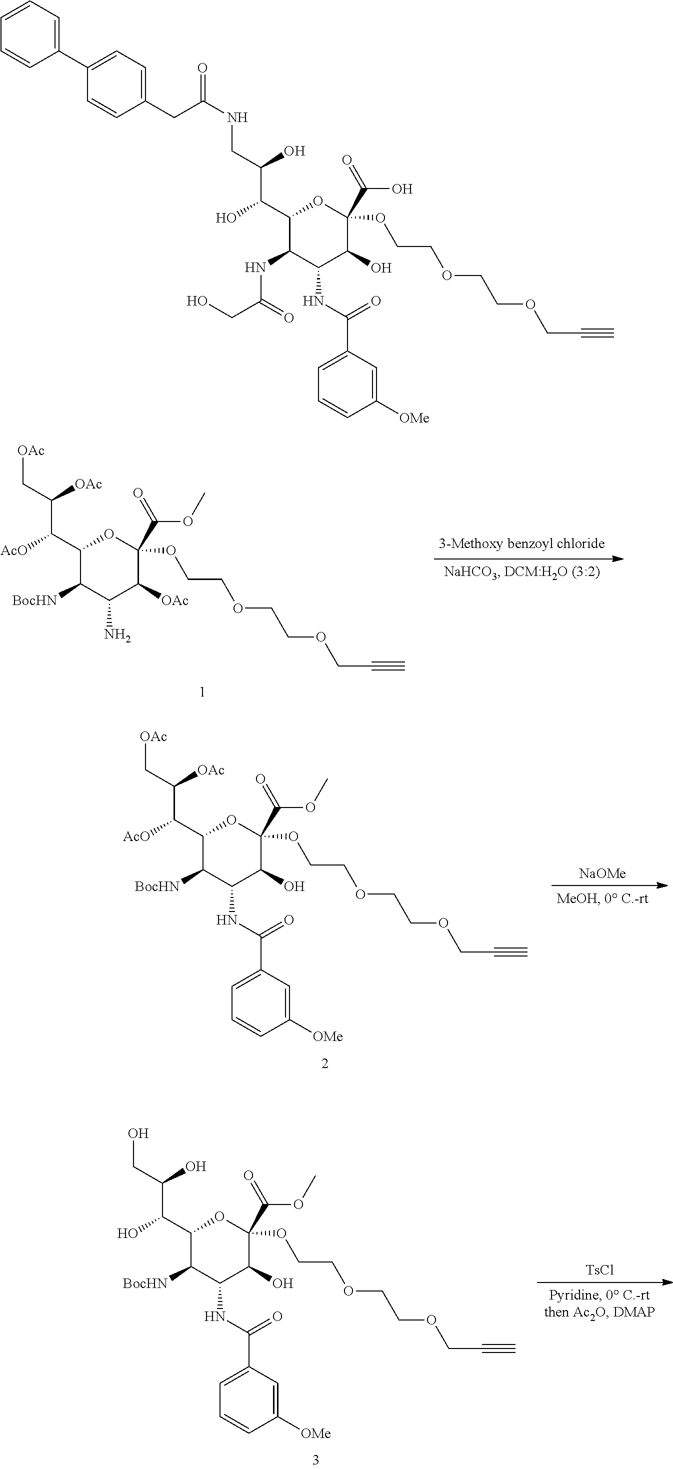

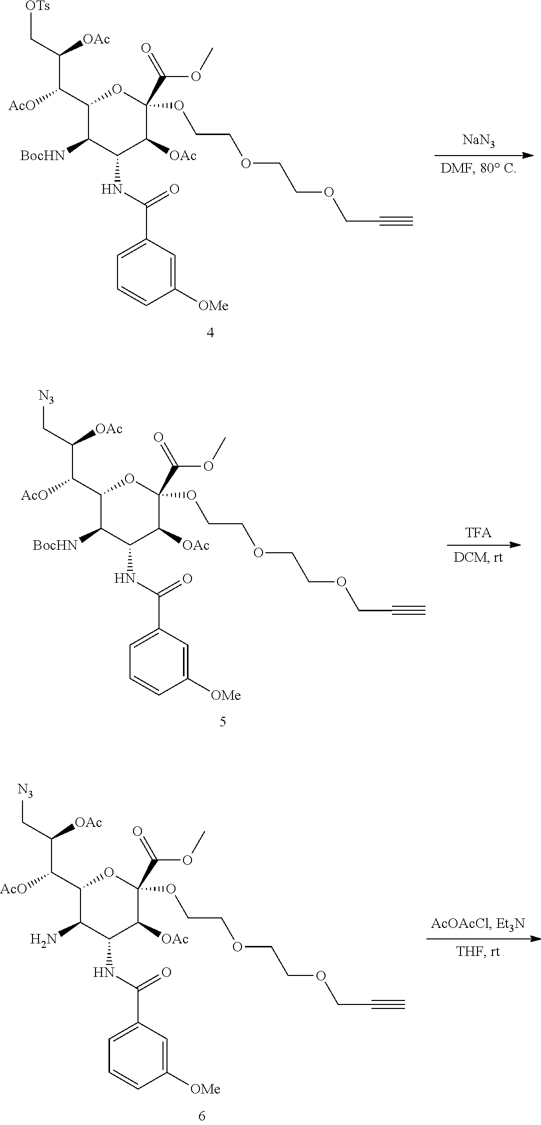

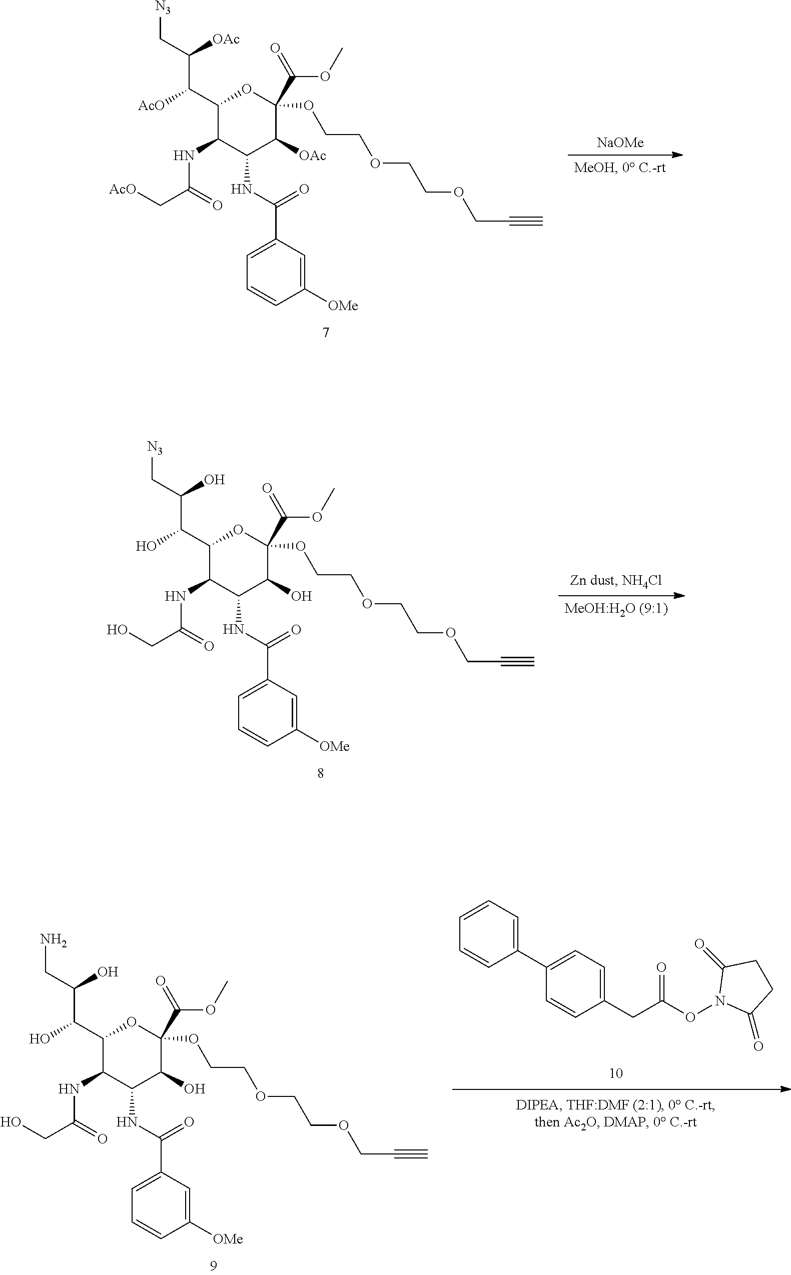

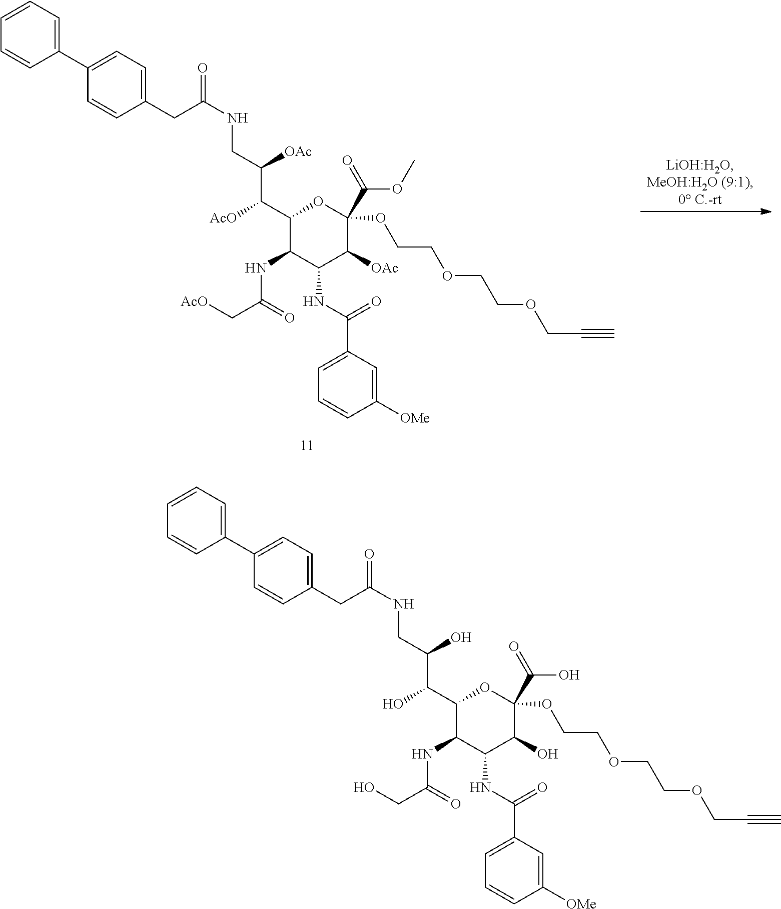 [SMILES: C#CCOCCOCCO[C@@]1(C(=O)OC)O[C@@H]([C@H](OC(C)=O)[C@@H](CNC(=O)Cc2ccc(-c3ccccc3)cc2)OC(C)=O)[C@H](NC(=O)COC(C)=O)[C@@H](NC(=O)c2cccc(OC)c2)[C@@H]1OC(C)=O ; "LiOH:H2O,MeOH:H2O (9:1),0° C.-rt" ; C#CCOCCOCCO[C@@]1(C(=O)O)O[C@@H]([C@H](O)[C@H](O)CNC(=O)Cc2ccc(-c3ccccc3)cc2)[C@H](NC(=O)CO)[C@@H](NC(=O)c2cccc(OC)c2)[C@@H]1O]

### Synthesis of (1S,2R)-1-((2R,3R,4R,5S,6R)-3-((tert-butoxycarbonyl)amino)-5-hydroxy-4-(3-methoxybenzamido)-6-(methoxycarbonyl)-6-(2-(2-(prop-2-yn-1-yloxy)ethoxy)ethoxy)tetrahydro-2H-pyran-2-yl)propane-1,2,3-triyl triacetate (2)

The crude (1S,2R)-1-((2R,3R,4R,5S,6R)-4-amino-3-((tert-butoxycarbonyl)amino)-5-hydroxy-6-(methoxycarbonyl)-6-(2-(2-(prop-2-yn-1-yloxy)ethoxy)ethoxy)tetrahydro-2H-pyran-2-yl)propane-1,2,3-triyl triacetate (1, 3.0 g, 4.62 mmol) was dissolved in dichloromethane and water (30 mL:20 mL) and cooled down to 0° C. Solid sodium bicarbonate (1.17 g, 13.9 mmol) and 3-methoxy benzoyl chloride chloride (1.21 mL, 9.25 mmol) were sequentially added to the reaction mixture at 0° C. The reaction mixture was stirred for 4 h then diluted with chloroform and washed with water. The aqueous layer was extracted twice with dichloromethane. The combined organic layers were washed with brine, dried, filtered, and the filtrate was concentrated. The crude product obtained was purified by silica gel column chromatography using methanol in dichloromethane as eluents to afford the desired product (2). Yield: 2.8 g, 77.3%; LCMS m/z 783.1 [M+H]⁺.

### Synthesis of methyl (2R,3S,4R,5R,6R)-5-((tert-butoxycarbonyl)amino)-3-hydroxy-4-(3-methoxybenzamido)-2-(2-(2-(prop-2-yn-1-yloxy)ethoxy)ethoxy)-6-((1R,2R)-1,2,3-trihydroxypropyl)tetrahydro-2H-pyran-2-carboxylate (3)

To a stirred solution of (1S,2R)-1-((2R,3R,4R,5S,6R)-3-((tert-butoxycarbonyl)amino)-5-hydroxy-4-(3-methoxybenzamido)-6-(methoxycarbonyl)-6-(2-(2-(prop-2-yn-1-yloxy)ethoxy)ethoxy)tetrahydro-2H-pyran-2-yl)propane-1,2,3-triyl triacetate (2, 2.8 g, 3.83 mmol) in methanol (50 mL) was added 25% sodium methoxide in methanol (0.02 mL, 0.38 mmol). The reaction mixture was stirred for 30 min at room temperature then neutralized with acidic resin (Dowex-Hydrogen form) until pH˜6, filtered, and the filtrate was concentrated, and the crude residue obtained was triturated with diethyl ether and dried under vacuum to afford the desired product (3) as a crude which was used for the next step without further purification. Yield: 2.0 g, Crude; LC-MS m/z 657.1 [M+H]⁺.

### Synthesis of (1S,2R)-1-((2R,3R,4R,5S,6R)-5-acetoxy-3-((tert-butoxycarbonyl)amino)-4-(3-methoxybenzamido)-6-(methoxycarbonyl)-6-(2-(2-(prop-2-yn-1-yloxy)ethoxy)ethoxy)tetrahydro-2H-pyran-2-yl)-3-(tosyloxy)propane-1,2-diyl diacetate (4)

To a stirred solution of methyl (2R,3S,4R,5R,6R)-5-((tert-butoxycarbonyl)amino)-3-hydroxy-4-(3-methoxybenzamido)-2-(2-(2-(prop-2-yn-1-yloxy)ethoxy)ethoxy)-6-((1R,2R)-1,2,3-trihydroxypropyl)tetrahydro-2H-pyran-2-carboxylate (3, 0.8 g, 1.22 mmol) in pyridine (10 mL) at 0° C., were sequentially added 4-methylbenzene-1-sulfonyl chloride (0.507 g, 2.66 mmol) and a catalytic amount of N,N-dimethyl-4-amino-pyridine (0.014 g, 0.121 mmol). The reaction mixture was stirred overnight from 0° C. to room temperature. After complete consumption of the starting material, acetic anhydride (3.0 mL) was added dropwise and the mixture was allowed to stir at room temperature overnight. The reaction mixture was concentrated, and the crude residue obtained was purified by silica gel column chromatography using MeOH in DCM to afford the desired product (4). Yield: 0.40 g, 35%; LCMS m/z 937.1 [M+H]⁺.

### Synthesis of (1R,2R)-1-((2R,3R,4R,5S,6R)-5-acetoxy-3-((tert-butoxycarbonyl)amino)-4-(3-methoxybenzamido)-6-(methoxycarbonyl)-6-(2-(2-(prop-2-yn-1-yloxy)ethoxy)ethoxy)tetrahydro-2H-pyran-2-yl)-3-azidopropane-1,2-diyl diacetate (5)

To a stirred solution of (1S,2R)-1-((2R,3R,4R,5S,6R)-5-acetoxy-3-((tert-butoxycarbonyl)amino)-4-(3-methoxybenzamido)-6-(methoxycarbonyl)-6-(2-(2-(prop-2-yn-1-yloxy)ethoxy)ethoxy)tetrahydro-2H-pyran-2-yl)-3-(tosyloxy)propane-1,2-diyl diacetate (4, 0.45 g, 0.48 mmol) in N,N dimethyl formamide (10 mL), was added sodium azide (0.55 g, 8.54 mmol) and the mixture was stirred at 80° C. for 12 h. After complete consumption of the starting material, the reaction mixture was concentrated to dryness, diluted with chloroform and washed with water. The aqueous layer was extracted twice with chloroform. The combined organic layer was washed with brine, dried, filtered, and the filtrate was concentrated, and the crude residue obtained was purified by silica gel column chromatography using methanol in dichloromethane as eluents to afford the desired product (5) (0.30 g, 87%). Yield: 0.3 g, 87%; LCMS m/z 808.1 [M+H]⁺.

### Synthesis of (1R,2R)-1-((2R,3R,4R,5S,6R)-5-acetoxy-3-amino-4-(3-methoxybenzamido)-6-(methoxycarbonyl)-6-(2-(2-(prop-2-yn-1-yloxy)ethoxy)ethoxy)tetrahydro-2H-pyran-2-yl)-3-azidopropane-1,2-diyl diacetate (6)

To a stirred solution of (1S,2R)-1-((2R,3R,4R,5S,6R)-5-acetoxy-3-((tert-butoxycarbonyl)amino)-4-(3-methoxybenzamido)-6-(methoxycarbonyl)-6-(2-(2-(prop-2-yn-1-yloxy)ethoxy)ethoxy)tetrahydro-2H-pyran-2-yl)-3-(tosyloxy)propane-1,2-diyl diacetate (5, 0.08 g, 0.01 mmol) in dichloromethane (5 mL), was added trifluoro acetic acid (0.007 mL, 0.001 mmol) dropwise at 0° C. The mixture was stirred for 1 h then evaporated. The residue obtained was co-evaporated with toluene 3-4 times then dried under high vacuum to afford the desired product (6) as a crude. Yield: 0.07 g, 99%; LCMS m/z 708.1 [M+H]⁺.

### Synthesis of (1R,2R)-1-((2R,3R,4R,5S,6R)-5-acetoxy-3-(2-acetoxyacetamido)-4-(3-methoxybenzamido)-6-(methoxycarbonyl)-6-(2-(2-(prop-2-yn-1-yloxy)ethoxy)ethoxy)tetrahydro-2H-pyran-2-yl)-3-azidopropane-1,2-diyl diacetate (7)

To a stirred solution of (1R,2R)-1-((2R,3R,4R,5S,6R)-5-acetoxy-3-amino-4-(3-methoxybenzamido)-6-(methoxycarbonyl)-6-(2-(2-(prop-2-yn-1-yloxy)ethoxy)ethoxy)tetrahydro-2H-pyran-2-yl)-3-azidopropane-1,2-diyl diacetate (6, 0.07 g, 0.09 mmol) in tetrahydrofuran (3 mL) was cooled to 0° C. and after 10 min, triethyl amine (0.07 mL, 0.09 mmol) and acetoxy acetyl chloride (0.01 mL, 0.09 mmol) were added dropwise. and the mixture was stirred at the same temperature for 1 h then diluted with ethyl acetate, washed with brine, dried, filtered, and the filtrate was concentrated, and the crude residue obtained was purified by silica gel column chromatography using methanol in dichloromethane as eluents to afford the desired product (7) (0.03 g, 37.55%). Yield: 0.03 g, 37.55%; LCMS m/z 808.1 [M+H]⁺.

### Synthesis of methyl (2R,3S,4R,5R,6R)-6-((1R,2R)-3-azido-1,2-dihydroxypropyl)-3-hydroxy-5-(2-hydroxyacetamido)-4-(3-methoxybenzamido)-2-(2-(2-(prop-2-yn-1-yloxy)ethoxy)ethoxy)tetrahydro-2H-pyran-2-carboxylate (8)

To a stirred solution of (1R,2R)-1-((2R,3R,4R,5S,6R)-5-acetoxy-3-(2-acetoxyacetamido)-4-(3-methoxybenzamido)-6-(methoxycarbonyl)-6-(2-(2-(prop-2-yn-1-yloxy)ethoxy)ethoxy)tetrahydro-2H-pyran-2-yl)-3-azidopropane-1,2-diyl diacetate (7, 0.16 g, 0.168 mmol) in methanol (10 mL) was added 25% sodium methoxide in methanol (0.02 mL, 0.02 mmol). The reaction mixture was stirred for 30 min at room temperature then neutralized with acidic resin (Dowex-Hydrogen form), filtered, and the filtrate was concentrated, and the crude residue obtained was triturated with diethyl ether and dried under vacuum to afford the desired product (8) as a crude which was used for next step without further purification. Yield: 0.09 g, 71%; Crude; LC-MS m/z 640.1 [M+H]⁺.

### Synthesis of methyl (2R,3S,4R,5R,6R)-6-((1R,2R)-3-amino-1,2-dihydroxypropyl)-3-hydroxy-5-(2-hydroxyacetamido)-4-(3-methoxybenzamido)-2-(2-(2-(prop-2-yn-1-yloxy)ethoxy)ethoxy)tetrahydro-2H-pyran-2-carboxylate (9)

To a stirred solution of methyl (2R,3S,4R,5R,6R)-6-((1R,2R)-3-azido-1,2-dihydroxypropyl)-3-hydroxy-5-(2-hydroxyacetamido)-4-(3-methoxybenzamido)-2-(2-(2-(prop-2-yn-1-yloxy)ethoxy)ethoxy)tetrahydro-2H-pyran-2-carboxylate (8, 0.09 g, 0.14 mmol) in methanol:water (9:1, 30 mL), zinc dust (0.09 g, 1.41 mmol) and ammonium chloride (0.07 g, 1.41 mmol) were added at 0° C. then stirred at room temperature for 1 h. After completion, the reaction mixture was filtered over celite, washed with methanol. The filtrate was concentrated to afford the desired product (9) which was used for the next reaction without further purification. Yield: 0.08 g, 92.65% Crude; LC-MS m/z 614.1 [M+H]⁺.

### Synthesis of methyl (2R,3S,4R,5R,6R)-6-((1R,2R)-3-(2-([1,1′-biphenyl]-4-yl)acetamido)-1,2-dihydroxypropyl)-3-hydroxy-5-(2-hydroxyacetamido)-4-(3-methoxybenzamido)-2-(2-(2-(prop-2-yn-1-yloxy)ethoxy)ethoxy)tetrahydro-2H-pyran-2-carboxylate (11)

To a stirred solution of crude methyl (2R,3S,4R,5R,6R)-6-((1R,2R)-3-amino-1,2-dihydroxypropyl)-3-hydroxy-5-(2-hydroxyacetamido)-4-(3-methoxybenzamido)-2-(2-(2-(prop-2-yn-1-yloxy)ethoxy)ethoxy)tetrahydro-2H-pyran-2-carboxylate (9, 1.00 g, 2.250 mmol) and 2,5-dioxopyrrolidin-1-yl 2-([1,1′-biphenyl]-4-yl)acetate (10, 0.659 g, 1.80 mmol) in tetrahydrofuran (8.00 mL) was added ethylbis(propan-2-yl)amine (1.96 mL, 11.20 mmol) at 0° C. then stirred at room temperature for h. 4 h. Acetic anhydride (2.13 mL, 22.50 mmol) and N, N-dimethylamino pyridine (0.137 g, 1.12 mmol) were added at 0° C. and the resulting mixture was stirred at room temperature for 12 h. h. After completion, the reaction mixture was diluted with water and ethyl acetate. The organic layer was separated, dried, filtered, concentrated, and the crude residue obtained was purified by silica gel column chromatography using ethyl acetate in hexane as eluents to afford the desired product (11). Yield: 0.60 g, 30.87%; LCMS (ESI) m/z 976.1 [M+H]⁺.

### Synthesis of (2R,3S,4R,5R,6R)-6-((1R,2R)-3-(2-([1,1′-biphenyl]-4-yl)acetamido)-1,2-dihydroxypropyl)-3-hydroxy-5-(2-hydroxyacetamido)-4-(3-methoxybenzamido)-2-(2-(2-(prop-2-yn-1-yloxy)ethoxy)ethoxy)tetrahydro-2H-pyran-2-carboxylic (Cpd. 36)

To a stirred solution of methyl (2R,3S,4R,5R,6R)-6-((1R,2R)-3-(2-([1,1′-biphenyl]-4-yl)acetamido)-1,2-dihydroxypropyl)-3-hydroxy-5-(2-hydroxyacetamido)-4-(3-methoxybenzamido)-2-(2-(2-(prop-2-yn-1-yloxy)ethoxy)ethoxy)tetrahydro-2H-pyran-2-carboxylate (11, 0.150 g, 0.154 mmol) in methanol (10.0 mL), was added a solution of lithium hydroxide monohydrate (0.022 g, 0.92 mmol) in water (0.5 mL). The reaction mixture was stirred at room temperature for 6 h. After completion, the reaction mixture was treated with acidic resin (Dowex 50H⁺) to pH˜6 and the suspension was filtered. The filtrate was concentrated, and the crude residue obtained was purified by prep HPLC using acetonitrile in water (+0.1% TFA) as eluents to afford the desired product (Cpd. 36). Yield: 0.60 g, 30.87%; LCMS (ESI) m/z 796.46 [M+H]⁺. ¹H-NMR (400 MHz, Methanol-d₄):δ 8.31 (d, J=8.0 Hz, 1H), 7.93 (d, J=8.0 Hz, 1H), (7.61-7.56 (m, 4H), 7.44-7.29 (m, 7H), 7.23 (d, J=8.0 Hz, 1H), 7.08 (d, J=8.0 Hz, 1H), 4.53-4.52 (m, 1H), 4.42-4.39 (m, 1H), 4.28-4.26 (m, 1H), 4.13 (d, J=2.4 Hz, 2H), 4.04-4.01 (m, 1H), 3.95-3.89 (m, 3H), 3.84 (s, 3H), 3.71-3.59 (m, 10H), 3.38 (d, J=8.8 Hz, 1H), 3.30-3.22 (m, 1H), 2.81 (t, J=2.4 Hz, 2.4 Hz, 1H), 2.37 (s, 1H).

### (2R,4S,5R,6R)-6-((1R,2R)-1,2-dihydroxy-3-(2-(p-tolyl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 175)

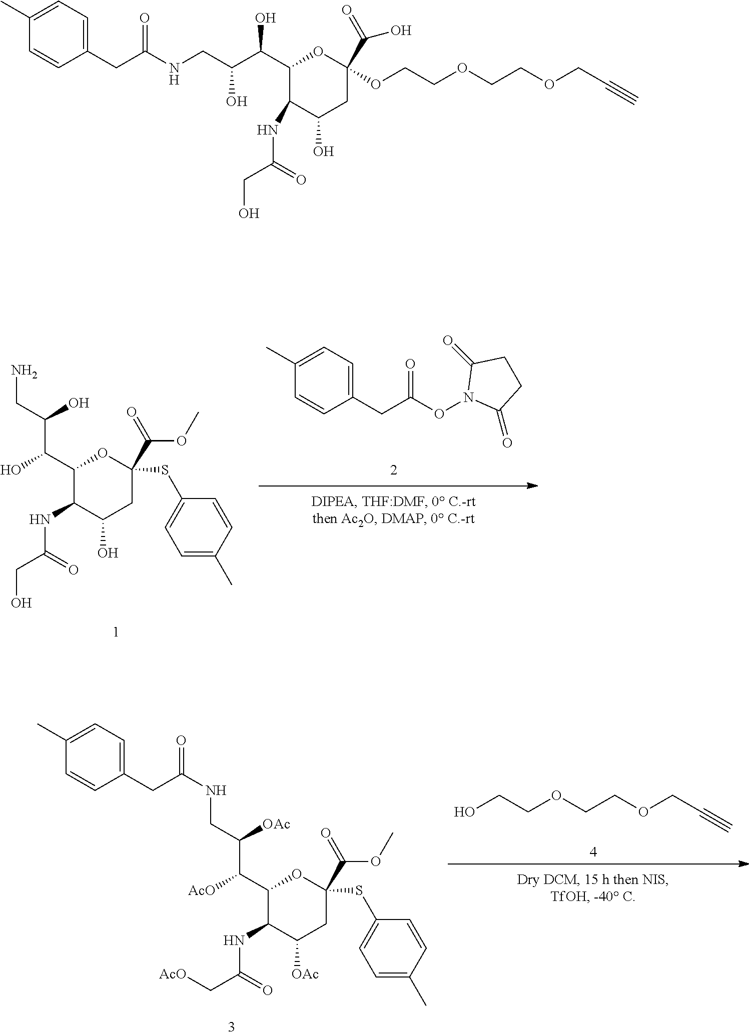

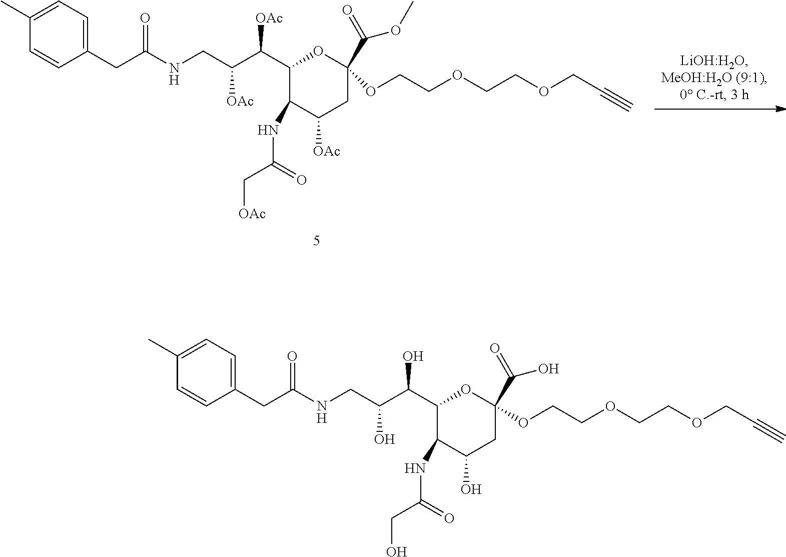

### Synthesis of (1R,2R)-1-((2R,3R,4S,6S)-4-acetoxy-3-(2-acetoxyacetamido)-6-(methoxycarbonyl)-6-(p-tolylthio)tetrahydro-2H-pyran-2-yl)-3-(2-(p-tolyl)acetamido)propane-1,2-diyl diacetate (3)

To a stirred solution of methyl (2S,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (1, 1.70 g, 3.83 mmol) and 2,5-dioxopyrrolidin-1-yl 2-(p-tolyl)acetate (2, 0.945 g, 3.83 mmol) in tetrahydrofuran (15.00 mL) and N,N-dimethylformamide (2.00 mL), was added ethylbis(propan-2-yl)amine (1.99 mL, 11.48 mmol) at 0° C. then stirred at room temperature for 4 h. Acetic anhydride (3.61 mL, 38.28 mmol) and 4-dimethylaminopyridine (0.046 g, 0.38 mmol) were added at 0° C. and the resulting mixture was stirred at room temperature for 12 h. After completion the mixture was diluted with water and ethyl acetate. The organic layer was separated, dried, filtered, concentrated, and the crude residue obtained was purified by silica gel column chromatography using ethyl acetate in hexane as eluents to afford the desired product (3). Yield: 1.20 g, 42.13%; LCMS (ESI) m/z 745.1 [M+H]⁺.

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(p-tolyl)acetamido)propane-1,2-diyl diacetate (5)

A mixture of (1R,2R)-1-((2R,3R,4S,6S)-4-acetoxy-3-(2-acetoxyacetamido)-6-(methoxycarbonyl)-6-(p-tolylthio)tetrahydro-2H-pyran-2-yl)-3-(2-(p-tolyl)acetamido)propane-1,2-diyl diacetate (3, 1.2 g, 1.24 mmol), 2-(2-(prop-2-yn-1-yloxy)ethoxy)ethan-1-ol (4, 0.568 g, 4.03 mmol) and activated powdered 4 Å molecular sieves (1.00 g, 100% w/w) in anhydrous dichloromethane (15.0 mL) was stirred at room temperature for 15 h. The solution was then cooled to −40° C. followed by addition of 1-iodopyrrolidine-2,5-dione (0.907 g, 4.03 mmol) and trifluoromethanesulfonic acid (0.23 mL, 1.61 mmol). The reaction mixture was stirred at this temperature for another 2 h. After completion, the reaction mixture was quenched by addition of triethyl amine (neutral pH) and warmed up gradually to room temperature. The reaction mixture was filtered, and the filtrate was washed with deionized water. The organic layer was separated, dried, filtered, concentrated, and the crude residue obtained was purified by silica gel column chromatography using ethyl acetate in hexane as eluents to afford the desired product (5). Yield: 0.70 g, 56.81%; LCMS m/z 765.05 [M+H]⁺.

### Synthesis of (2R,4S,5R,6R)-6-((1R,2R)-1,2-dihydroxy-3-(2-(p-tolyl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 175)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(p-tolyl)acetamido)propane-1,2-diyl diacetate (5, 0.70 g, 0.92 mmol) in methanol (10.0 mL), was added a solution of lithium hydroxide monohydrate (0.225 g, 5.49 mmol) in water (1.0 mL). The reaction mixture was stirred at room temperature for 4 h. After completion, the reaction mixture was treated with acidic resin (Dowex 50H⁺) until pH˜6 and the suspension was filtered. The filtrate was concentrated, and the crude residue obtained was purified by prep HPLC using acetonitrile and water (+0.1% TFA) as eluents to afford the desired product (Cpd. 175). Yield: 0.058 g, 10.18%; LCMS m/z 584.4 [M+H]⁺; ¹H NMR (400 MHz, methanol-d4) (7.86 (d, J=8.0 Hz, NH), 7.18-7.11 (m, 4H), 4.18 (d, J=2.4 Hz, 2H), 4.00 (s, 2H), 3.92-3.78 (m, 4H), 3.72 (d, J=10.0 Hz, 1H), 3.68-3.56 (m, 8H), 3.49 (s, 2H), 3.36 (dd, J=8.8 & 1.2 Hz, 1H), 3.26-3.23 (m, 1H), 2.84 (t, J=2.4 Hz, 2.4 Hz, 1H), 2.72 (dd, J=14.0 & 4.8 Hz, 1H), 2.30 (s, 3H), 1.76 (t, J=12.0 Hz, 12.0 Hz, 1H).

### (2R,4S,5R,6R)-6-((1R,2R)-3-(2-(4-chlorophenyl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 126)

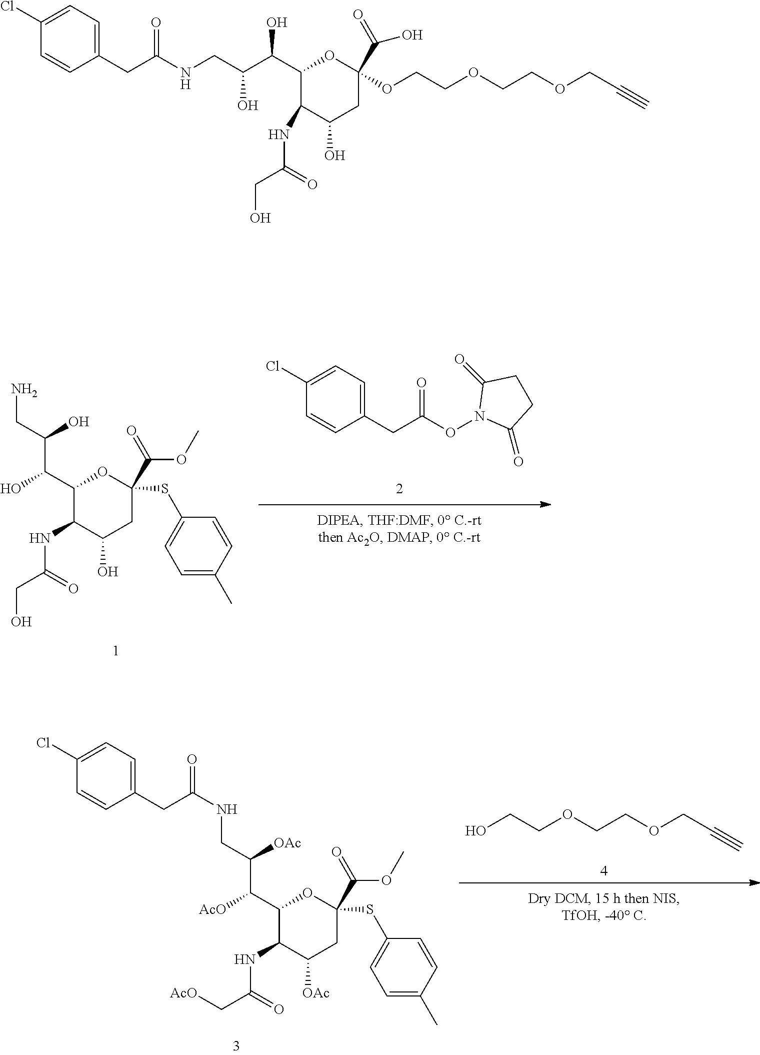

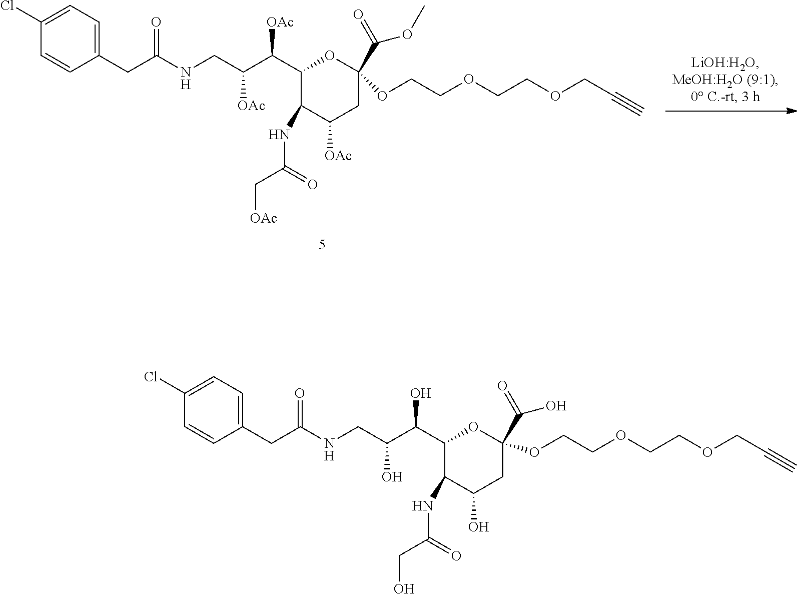 [SMILES: C#CCOCCOCCO[C@]1(C(=O)OC)C[C@H](OC(C)=O)[C@@H](NC(=O)COC(C)=O)[C@H]([C@H](OC(C)=O)[C@@H](CNC(=O)Cc2ccc(Cl)cc2)OC(C)=O)O1 ; "LiOH:H2O,MeOH:H2O (9:1),0° C.-rt, 3 h" ; C#CCOCCOCCO[C@]1(C(=O)O)C[C@H](O)[C@@H](NC(=O)CO)[C@H]([C@H](O)[C@H](O)CNC(=O)Cc2ccc(Cl)cc2)O1]

### Synthesis of (1R,2R)-1-((2R,3R,4S,6S)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (3)

To a stirred solution of methyl (2S,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (1, 1.70 g, 3.83 mmol) and 2,5-dioxopyrrolidin-1-yl 2-(4-chlorophenyl)acetate (2, 1.02 g, 3.83 mmol) in tetrahydrofuran (15.0 mL) and N,N-dimethylformamide (2.00 mL) was added ethylbis(propan-2-yl)amine (2.06 mL, 11.48 mmol) at 0° C. then stirred at room temperature for 4 h. Acetic anhydride (3.61 mL, 38.28 mmol) and 4-dimethylaminopyridine (0.046 g, 0.38 mmol) were added at 0° C. and the resulting mixture was stirred at room temperature for 12 h. After completion the mixture was diluted with water and ethyl acetate. The organic layer was separated, dried, filtered, concentrated, and the crude residue obtained was purified by silica gel column chromatography using ethyl acetate in hexane as eluents to afford the desired product (3). Yield: 1.10 g, 37.59%; LCMS (ESI) m/z 766.12 [M+H]⁺.

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(4-chlorophenyl)acetamido)propane-1,2-diyl diacetate (5)

A mixture of (1R,2R)-1-((2R,3R,4S,6S)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (3, 1.10 g, 1.44 mmol), 2-(2-(prop-2-yn-1-yloxy)ethoxy)ethan-1-ol (4, 0.507 g, 3.56 mmol) and activated powdered 4 Å molecular sieves (1.00 g, 100% w/w) in anhydrous dichloromethane (15.0 mL) was stirred at room temperature for 15 h. The solution was then cooled to −40° C. followed by addition of 1-iodopyrrolidine-2,5-dione (0.810 g, 3.61 mmol) and trifluoromethanesulfonic acid (0.20 mL, 1.44 mmol). The reaction mixture was stirred at this temperature for another 2 h. After completion, the reaction mixture was quenched by addition of triethyl amine (neutral pH) and warmed up gradually to room temperature. The reaction mixture was filtered, and the filtrate was washed with deionized water. The organic layer was separated, dried, filtered, concentrated, and the crude residue obtained was purified by silica gel column chromatography using ethyl acetate in hexane as eluents to afford the desired product (5) as a mixture of anomers. Yield: 0.80 g, 70.88%; LCMS m/z 785.10 [M+H]⁺.

### Synthesis of (2R,4S,5R,6R)-6-((1R,2R)-3-(2-(4-chlorophenyl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 126)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(4-chlorophenyl)acetamido)propane-1,2-diyl diacetate (5, 0.80 g, 1.02 mmol) in methanol (10.0 mL), was added a solution of lithium hydroxide monohydrate (0.250 g, 6.12 mmol) in water (1.0 mL). The reaction mixture was stirred at room temperature for 4 h. After completion, the reaction mixture was treated with acidic resin (Dowex 50H⁺) until pH˜6 and the suspension was filtered. The filtrate was concentrated, and the crude residue obtained was purified by prep HPLC using acetonitrile and water (+0.1% TFA) as eluents to afford the desired product (Cpd. 126). Yield: 0.141 g, 22.95%; LCMS m/z 603.35 [M+H]⁺; ¹H NMR (400 MHz, methanol-d₄) δ 7.83 (d, J=8.8 Hz, NH), 7.32-7.27 (m, 4H), 4.18 (d, J=2.4 Hz, 2H), 4.01 (s, 2H), 3.92-3.80 (m, 4H), 3.72 (d, J=10.0 Hz, 1H), 3.68-3.57 (m, 8H), 3.53 (s, 2H), 3.36 (dd, J=8.8 & 1.2 Hz, 1H), 3.26-3.23 (m, 1H), 2.84 (t, J=2.4 Hz, 2.4 Hz, 1H), 2.72 (dd, J=14.0 & 4.8 Hz, 1H), 1.76 (t, J=12.0 Hz, 12.0 Hz, 1H).

### (2R,4S,5R,6R)-6-((1R,2R)-1,2-dihydroxy-3-(2-(4-methoxyphenyl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 174)

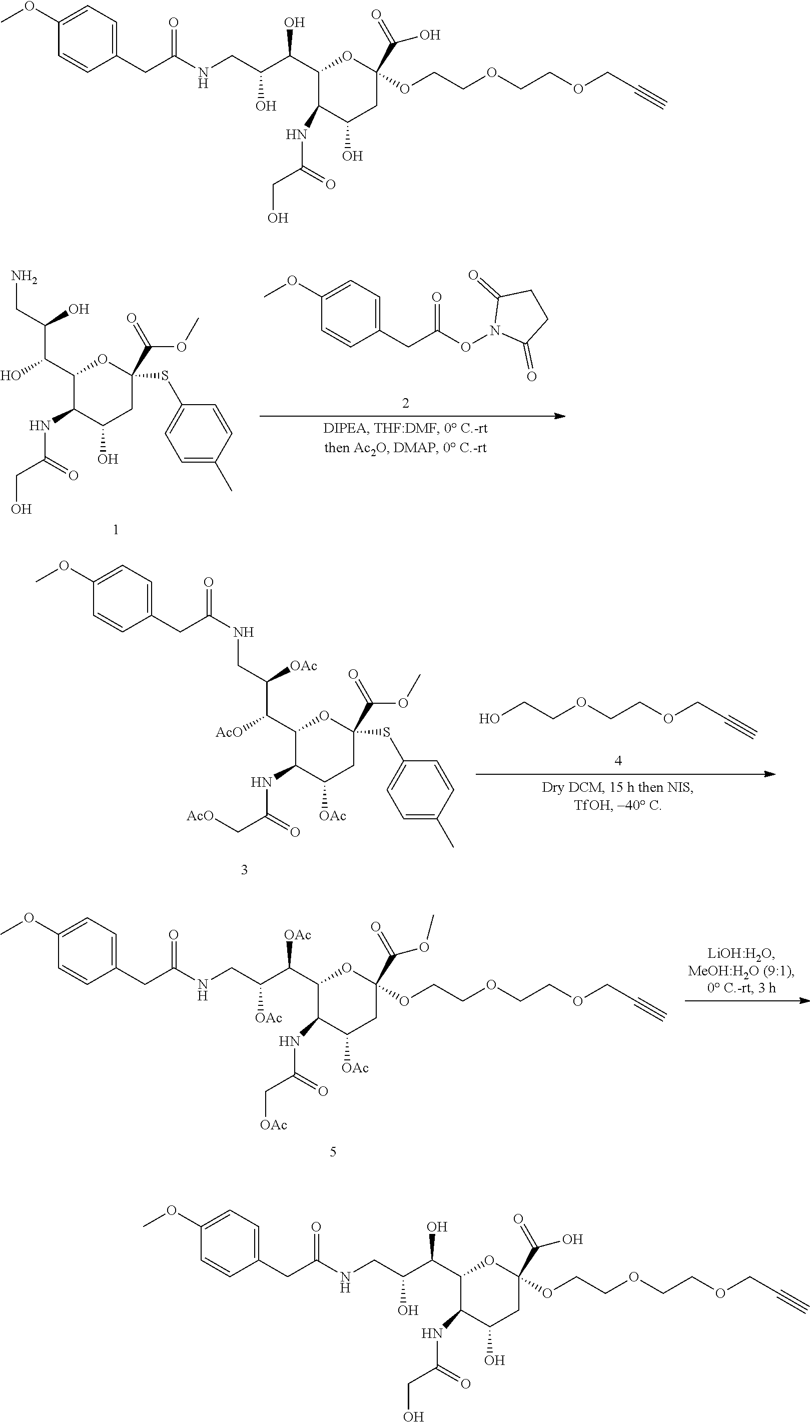

### Synthesis of (1R,2R)-1-((2R,3R,4S,6S)-4-acetoxy-3-(2-acetoxyacetamido)-6-(methoxycarbonyl)-6-(p-tolylthio)tetrahydro-2H-pyran-2-yl)-3-(2-(4-methoxyphenyl)acetamido)propane-1,2-diyl diacetate (3)

To a stirred solution of methyl (2S,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (1, 1.0 g, 2.25 mmol) and 2,5-dioxopyrrolidin-1-yl 2-(4-methoxyphenyl)acetate (2, 0.592 g, 2.25 mmol) in tetrahydrofuran (15.0 mL) and N,N-dimethylformamide (2.00 mL) was added ethylbis(propan-2-yl)amine (1.18 mL, 6.75 mmol) at 0° C. then stirred at room temperature for 4 h. Acetic anhydride (2.13 mL, 22.50 mmol) and 4-dimethylaminopyridine (0.027 g, 0.22 mmol) was added at 0° C. and the resulting mixture was stirred at room temperature for 12 h. After completion, the mixture was diluted with water and ethyl acetate. The organic layer was separated, dried, filtered, concentrated, and the crude residue obtained was purified by silica gel column chromatography using ethyl acetate in hexane as eluents to afford the desired product (3). Yield: 0.95 g, 55.50%; LCMS (ESI) m/z 761.90 [M+H]⁺.

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(4-methoxyphenyl)acetamido)propane-1,2-diyl diacetate (5)

A mixture of (1R,2R)-1-((2R,3R,4S,6S)-4-acetoxy-3-(2-acetoxyacetamido)-6-(methoxycarbonyl)-6-(p-tolylthio)tetrahydro-2H-pyran-2-yl)-3-(2-(4-methoxyphenyl)acetamido)propane-1,2-diyl diacetate (3, 0.95 g, 1.25 mmol), 2-(2-(prop-2-yn-1-yloxy)ethoxy)ethan-1-ol (4, 0.45 g, 3.12 mmol) and activated powdered 4 Å molecular sieves (1.00 g, 100% w/w) in anhydrous dichloromethane (15.0 mL) was stirred at room temperature for 15 h. The solution was then cooled to −40° C. followed by addition of 1-iodopyrrolidine-2,5-dione (0.674 g, 3.00 mmol) and trifluoromethanesulfonic acid (0.22 mL, 1.25 mmol). The reaction mixture was stirred at this temperature for another 2 h. After completion, the reaction mixture was quenched by addition of triethyl amine (neutral pH) and warmed up gradually to room temperature. The reaction mixture was filtered, and the filtrate was washed with deionized water. The organic layer was separated, dried, filtered, concentrated, and the crude residue obtained was purified by silica gel column chromatography using ethyl acetate in hexane as eluents to afford the desired product (5). Yield: 0.72 g, 73.85%; LCMS m/z 781.10 [M+H]⁺.

### Synthesis of (2R,4S,5R,6R)-6-((1R,2R)-1,2-dihydroxy-3-(2-(4-methoxyphenyl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 174)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(4-methoxyphenyl)acetamido)propane-1,2-diyl diacetate (5, 0.72 g, 0.922 mmol) in methanol (10.0 mL), was added a solution of lithium hydroxide monohydrate (0.226 g, 5.53 mmol) in water (1.0 mL). The reaction mixture was stirred at room temperature for 3 h. After completion, the reaction mixture was treated with acidic resin (Dowex 50H⁺) until pH˜6 and the suspension was filtered. The filtrate was concentrated, and the crude residue obtained was purified by prep HPLC using acetonitrile and water (+0.1% TFA) as eluents to afford the desired product (Cpd. 174). Yield: 0.049 g, 8.88%; LCMS m/z 599.45 [M+H]⁺; ¹H NMR (400 MHz, methanol-d₄) δ 7.21-7.19 (m, 2H), 7.88-6.84 (m, 2H), 4.18 (d, J=2.4 Hz, 2H), 4.01 (s, 2H), 3.94-3.80 (m, 4H), 3.77 (s, 3H), 3.71 (m, 1H), 3.68-3.56 (m, 8H), 3.46 (s, 2H), 3.35 (dd, J=8.8 & 1.2 Hz, 1H), 3.26-3.23 (m, 1H), 2.83 (t, J=2.4 Hz, 2.4 Hz, 1H), 2.72 (dd, J=12.4 & 4.8 Hz, 1H), 1.74 (t, J=12.4 Hz, 1H).

### (2R,4S,5R,6R)-6-((1R,2R)-1,2-dihydroxy-3-(2-(4′-hydroxy-[1,1′-biphenyl]-4-yl)acetamido)propyl)-5-(2-fluoroacetamido)-4-hydroxy-2-(2-(2-(prop-2-yn-1-yloxy)ethoxy)ethoxy)tetrahydro-2H-pyran-2-carboxylic acid (Compound 35)

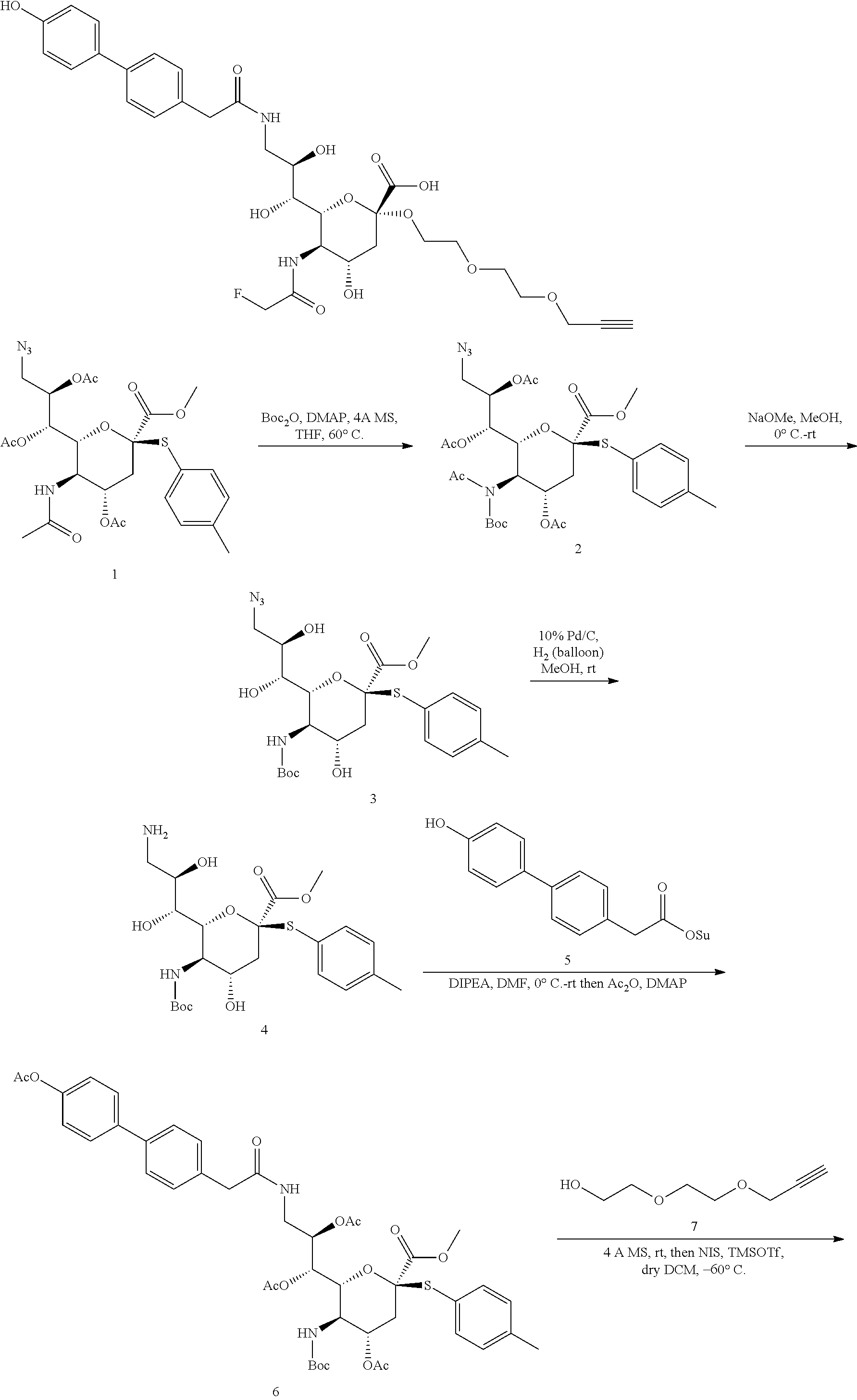

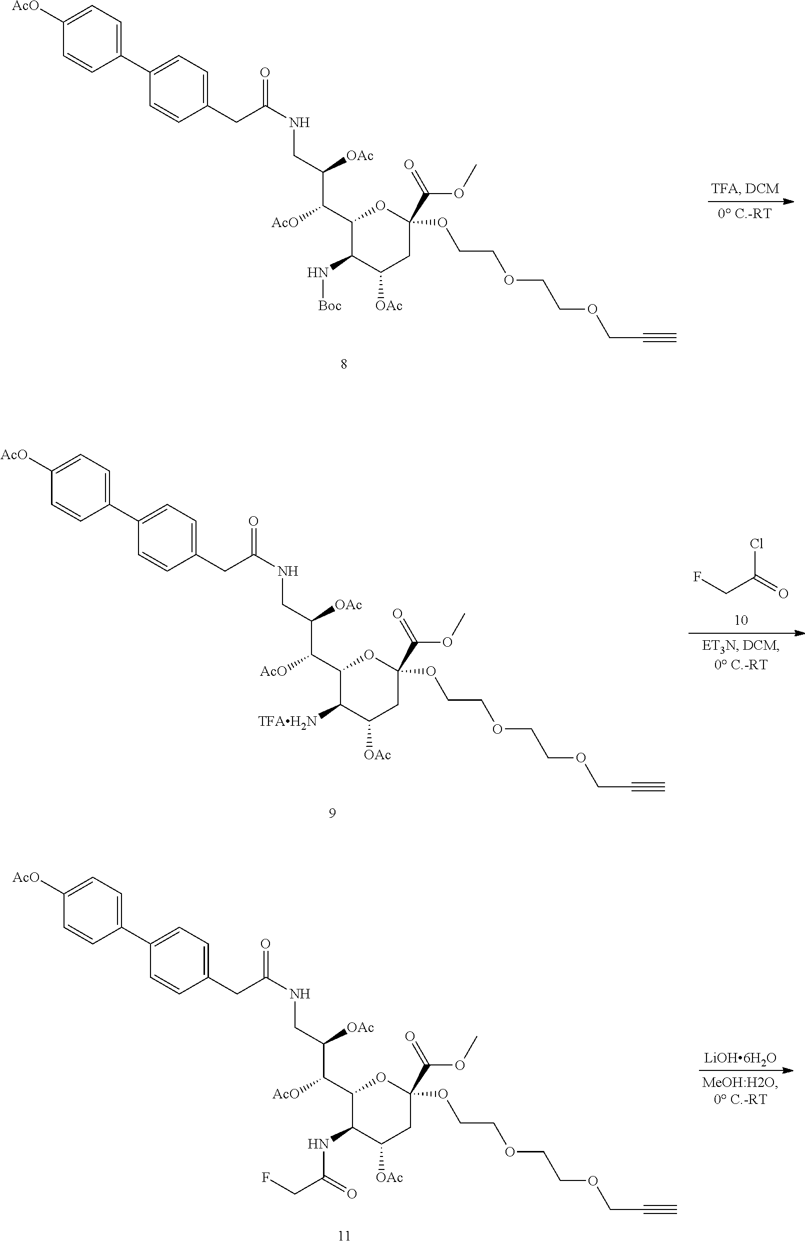

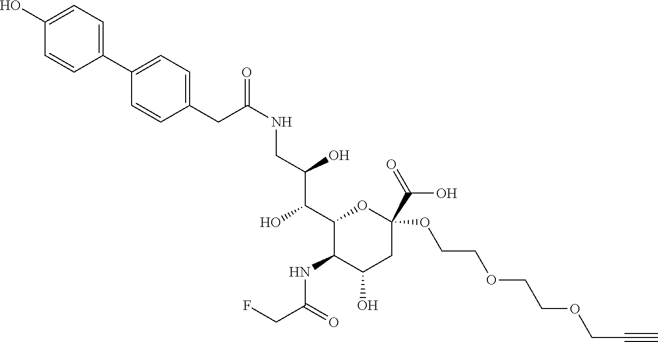 [SMILES: C#CCOCCOCCO[C@]1(C(=O)O)C[C@H](O)[C@@H](NC(=O)CF)[C@H]([C@H](O)[C@H](O)CNC(=O)Cc2ccc(-c3ccc(O)cc3)cc2)O1]

### Synthesis of (1R,2R)-1-((2R,3R,4S,6R)-4-acetoxy-3-(N-(tert-butoxycarbonyl)acetamido)-6-(methoxycarbonyl)-6-(p-tolylthio)tetrahydro-2H-pyran-2-yl)-3-azidopropane-1,2-diyl diacetate (2)

To a solution of (1R,2R)-1-((2R,3R,4S,6R)-3-acetamido-4-acetoxy-6-(methoxycarbonyl)-6-(p-tolylthio)tetrahydro-2H-pyran-2-yl)-3-azidopropane-1,2-diyl diacetate (1, 9.800 g, 16.90 mmol) and 4A molecular sieves (10.000 g) in THF (100.0 mL), were sequentially added Di-tert-butyl dicarbonate (20.5 mL, 84.40 mmol) and 4-Dimethylaminopyridine (6.190 g, 50.00 mmol) at room temperature. The resulting solution was stirred at room temperature for 10 min at then for 12 h at 65° C. After completion, the reaction mixture was concentrated, and the residue obtained was dissolved in ethyl acetate. The organic layer was washed with brine, dried and concentrated, and the crude residue obtained was purified by silica gel column chromatography using ethyl acetate in hexane as eluents to afford the desired product (2). Yield: 6.000 g, 52%; LCMS m/z 698.05 [M+H3O]⁺.

### Synthesis of methyl (2R,4S,5R,6R)-6-((1R,2R)-3-azido-1,2-dihydroxypropyl)-5-((tert-butoxycarbonyl)amino)-4-hydroxy-2-(p-tolylthio)tetrahydro-2H-pyran-2-carboxylate (3)

methyl (1R,2R)-1-((2R,3R,4S,6R)-4-acetoxy-3-(N-(tert-butoxycarbonyl)acetamido)-6-(methoxycarbonyl)-6-(p-tolylthio)tetrahydro-2H-pyran-2-yl)-3-azidopropane-1,2-diyl diacetate (2, 6.000 g, 8.81 mmol) was dissolved in methanol (60.0 mL) was added 25% sodium methoxide in methanol (1.14 mL, 5.29 mmol, pH should not be more than 9). The reaction mixture was stirred for 30 min at room temperature then neutralized with acidic resin (Dowex-Hydrogen form), filtered, and the filtrate was concentrated to afford the desired product (3). The crude residue obtained was used for next step without further purification. Yield: 4.300 g, Crude; LCMS m/z 530.00 [M+H3O]⁺.

### Synthesis of methyl (2R,4S,5R,6R)-6-((1R,2R)-3-amino-1,2-dihydroxypropyl)-5-((tert-butoxycarbonyl)amino)-4-hydroxy-2-(p-tolylthio)tetrahydro-2H-pyran-2-carboxylate (4)

To a stirred solution of methyl (2R,4S,5R,6R)-6-((1R,2R)-3-azido-1,2-dihydroxypropyl)-5-((tert-butoxycarbonyl)amino)-4-hydroxy-2-(p-tolylthio)tetrahydro-2H-pyran-2-carboxylate (3, 4.300 g, 8.39 mmol) in methanol (50.0 mL), was added palladium on carbon (4.000 g) at room temperature under argon. The resulting mixture was stirred for 16 h under 1 atm of H2 gas. The catalyst was filtered off through celite and the filtrate was concentrated to afford the desired product (4) as a crude which was used for the next step without further purification. Yield: 4.2 g, Crude; LCMS m/z 487.10 [M+H]⁺.

### Synthesis of (1R,2R)-1-((2R,3R,4S,6R)-4-acetoxy-3-((tert-butoxycarbonyl)amino)-6-(methoxycarbonyl)-6-(p-tolylthio)tetrahydro-2H-pyran-2-yl)-3-(2-(4′-acetoxy-[1,1′-biphenyl]-4-yl)acetamido)propane-1,2-diyl diacetate (6)

To a solution of methyl (2R,4S,5R,6R)-6-((1R,2R)-3-amino-1,2-dihydroxypropyl)-5-((tert-butoxycarbonyl)amino)-4-hydroxy-2-(p-tolylthio)tetrahydro-2H-pyran-2-carboxylate (4, 4.200 g, 8.63 mmol) and 2,5-dioxopyrrolidin-1-yl 2-(4′-hydroxy-[1,1′-biphenyl]-4-yl)acetate (5, 3.370 g, 10.40 mmol) in tetrahydrofuran (10.0 mL), was added ethylbis(propan-2-yl)amine (7.5 mL, 43.20 mmol) at 0° C. then stirred at room temperature for 4 h. After completion, acetic anhydride (6.5 mL, 69.04 mmol) and 4-dimethylaminopyridine (1.054 g, 8.63 mmol) were sequentially added at 0° C. and the resulting mixture was stirred at room temperature for 12 h. After completion, the reaction mixture was diluted with ethyl acetate and washed with water. The organic layer was separated, dried, filtered, and concentrated, and the crude residue was purified by silica gel column chromatography using ethyl acetate in hexane as eluents to afford the desired product (6). Yield: 4.0 g, 53%; LCMS m/z 866.00 [M+H]⁺.

### Synthesis of (1R,2R)-1-((2R,3R,4S,6R)-4-acetoxy-3-((tert-butoxycarbonyl)amino)-6-(methoxycarbonyl)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (8)

A mixture of (1R,2R)-1-((2R,3R,4S,6R)-4-acetoxy-3-((tert-butoxycarbonyl)amino)-6-(methoxycarbonyl)-6-(p-tolylthio)tetrahydro-2H-pyran-2-yl)-3-(2-(4′-acetoxy-[1,1′-biphenyl]-4-yl)acetamido)propane-1,2-diyl diacetate (6, 1.800 g, 2.08 mmol), 2-(2-(prop-2-yn-1-yloxy)ethoxy)ethan-1-ol (7, 1.5 g, 10.40 mmol) and activated powdered 4 Å molecular sieves (1.8 g, 100% w/w) in anhydrous dichloromethane (20.0 mL) was stirred at room temperature for 15 h. The solution was then cooled to −40° C. followed by addition of 1-iodopyrrolidine-2,5-dione (1.17 g, 5.20 mmol) and trimethylsilyl trifluoromethanesulfonate (0.455 mL, 2.5 mmol). The reaction mixture was stirred at this temperature for 1 h then quenched by addition of triethylamine (0.5 mL) and warmed up gradually to room temperature. The reaction mixture was filtered, and the filtrate was washed with saturated aqueous sodium thiosulfate solution. The organic layer was separated, dried, filtered, concentrated, and the crude residue obtained was purified by silica gel column chromatography using ethyl acetate in hexane as eluents to afford the desired product (8). Yield: 1.400 g, 76%; LCMS m/z 887.05 [M+H]⁺.

### Synthesis of (1R,2R)-1-((2R,3R,4S,6R)-4-acetoxy-3-amino-6-(methoxycarbonyl)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (9)

To a solution of (1R,2R)-1-((2R,3R,4S,6R)-4-acetoxy-3-((tert-butoxycarbonyl)amino)-6-(methoxycarbonyl)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (8, 0.30 g, 0.34 mmol), in anhydrous dichloromethane (0.5 mL), trifluoroacetic acid (0.5 mL) was added at 0° C., was stirred at room temperature for 4 h. After completion, the solvent was concentrated to afford the desired product (9). The crude residue obtained was used for the next step without further purification. Yield: 0.260 g, Crude; LCMS (ESI) m/z 785.25 [M+H]⁺.

### Synthesis of (1R,2R)-1-((2R,3R,4S,6R)-4-acetoxy-3-(2-fluoroacetamido)-6-(methoxycarbonyl)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (11)

To a solution of (1R,2R)-1-((2R,3R,4S,6R)-4-acetoxy-3-amino-6-(methoxycarbonyl)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (9, 0.20 g, 0.25 mmol) in dichloromethane (2.0 mL), were added triethylamine (0.107 mL, 0.76 mmol) followed by 2-fluoroacetyl chloride (10, 0.032 g, 0.33 mmol) dropwise at 0° C. The reaction was stirred at 0° C. a for 7 h then concentrated and the crude residue obtained was purified by silica gel column chromatography using ethyl acetate in hexane as eluents to afford the desired product (11). Yield: 0.20 g, 92.85%; LCMS (ESI) m/z 842.95 [M−H]⁻.

### Synthesis of (2R,4S,5R,6R)-6-((1R,2R)-1,2-dihydroxy-3-(2-(4′-hydroxy-[1,1′-biphenyl]-4-yl)acetamido)propyl)-5-(2-fluoroacetamido)-4-hydroxy-2-(2-(2-(prop-2-yn-1-yloxy)ethoxy)ethoxy)tetrahydro-2H-pyran-2-carboxylic acid (Cpd. 35)

To a stirred solution of (1R,2R)-1-((2R,3R,4S,6R)-4-acetoxy-3-(2-fluoroacetamido)-6-(methoxycarbonyl)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (11, 0.20 g, 0.24 mmol) in methanol (5 mL), was added a solution of lithium hydroxide monohydrate (0.034 g, 1.42 mmol) in water (1.0 mL). The reaction mixture was stirred at room temperature for 6 h. After completion, the reaction mixture was treated with acidic resin (Dowex 50, H⁺) until pH ˜6 and the suspension was filtered. The filtrate was concentrated, and the crude residue obtained was purified by prep HPLC using acetonitrile in water (+0.1% FA) to afford the desired product (Cpd. 35). Yield: 0.079 g, 50.36%; LCMS m/z 663.35 [M+H]⁺; ¹H NMR (400 MHz, methanol-d₄) δ 7.51 (d, J=8.4 Hz, 2H), 7.45-7.42 (m, 2H), 7.34 (d, J=8.0 Hz, 2H), 6.85-6.83 (m, 2H), 4.73 (m, 1H), 4.17 (d, J=2.4 Hz, 2H), 3.92-3.56 (m, 16 h), 3.39-3.37 (m, 1H), 3.31 (m, 1H), 2.83 (t, J=2.4 Hz, 1H), 2.73-2.69 (dd, J=12.8, 4.8 Hz, 1H), 1.78 (t, J=12.4 Hz, 1H).

### (2R,4S,5R,6R)-6-((1R,2R)-1,2-dihydroxy-3-(2-(3′-hydroxy-[1,1′-biphenyl]-4-yl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 78)

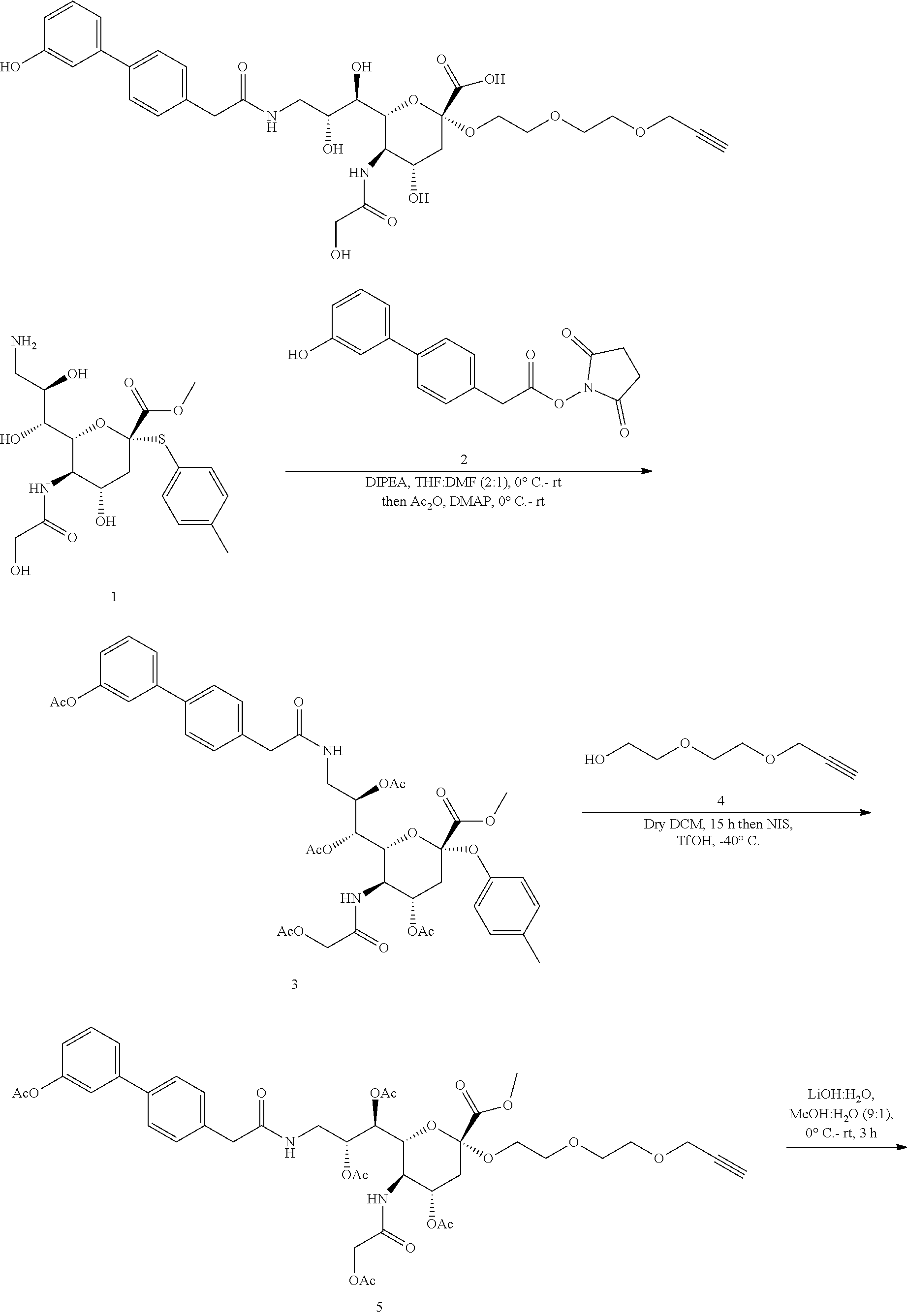

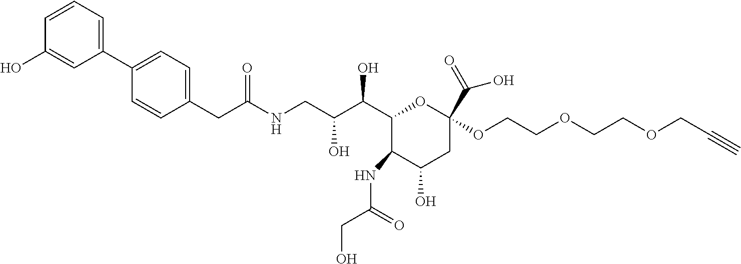 [SMILES: C#CCOCCOCCO[C@]1(C(=O)O)C[C@H](O)[C@@H](NC(=O)CO)[C@H]([C@H](O)[C@H](O)CNC(=O)Cc2ccc(-c3cccc(O)c3)cc2)O1]

### Synthesis of (1R,2R)-1-((2R,3R,4S,6S)-4-acetoxy-3-(2-acetoxyacetamido)-6-(methoxycarbonyl)-6-(p-tolylthio)tetrahydro-2H-pyran-2-yl)-3-(2-(3′-hydroxy-[1,1′-biphenyl]-4-yl)acetamido)propane-1,2-diyl diacetate (3)

To a solution of methyl (2S,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (1, 1.0 g, 2.25 mmol) in tetrahydrofuran (20.0 mL) was added 2,5-dioxopyrrolidin-1-yl 2-(3′-hydroxy-[1,1′-biphenyl]-4-yl)acetate (0.586 g, 1.8 mmol) followed by ethylbis(propan-2-yl)amine (1.96 mL, 11.2 mmol) at 0° C. then stirred at room temperature for 6 h. After completion, acetic anhydride (2.13 mL, 22.50 mmol) and 4-dimethylaminopyridine (0.027 g, 0.22 mmol) were added at 0° C. and the resulting mixture was stirred at room temperature for another 12 h. The reaction mixture was concentrated, and the crude residue obtained was purified by silica gel column chromatography using ethyl acetate in hexane as eluents to afford the desired product (3). Yield: 0.80 g, 72.5%; LCMS (ESI) m/z 865.3 [M+H]⁺.

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(3′-acetoxy-[1,1′-biphenyl]-4-yl)acetamido)propane-1,2-diyl diacetate (5)

A mixture of (1R,2R)-1-((2R,3R,4S,6S)-4-acetoxy-3-(2-acetoxyacetamido)-6-(methoxycarbonyl)-6-(p-tolylthio)tetrahydro-2H-pyran-2-yl)-3-(2-(3′-hydroxy-[1,1′-biphenyl]-4-yl)acetamido)propane-1,2-diyl diacetate (4, 0.80 g, 0.924 mmol), 2-(2-(prop-2-yn-1-yloxy)ethoxy)ethan-1-ol (5, 0.666 g, 4.62 mmol) and activated powdered 4 Å molecular sieves (1.00 g, 100% w/w) in anhydrous dichloromethane (20.0 mL) was stirred at room temperature for 15 h. The solution was then cooled to −40° C. followed by addition of 1-iodopyrrolidine-2,5-dione (0.520 g, 2.32 mmol) and trifluoromethanesulfonic acid (0.081 mL, 0.924 mmol). The reaction mixture was stirred at this temperature for another 1 h. After completion, the reaction mixture was quenched by addition of triethyl amine (neutral pH) and warmed up gradually to room temperature. The reaction mixture was filtered, and the filtrate was washed with deionized water. The organic layer was separated, dried, filtered, concentrated, and the crude residue obtained was purified by silica gel column chromatography using ethyl acetate in hexane as eluents to afford the desired product (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(3′-acetoxy-[1,1′-biphenyl]-4-yl)acetamido)propane-1,2-diyl diacetate (5). Yield: 0.70 g, 85%; LCMS m/z 885.35 [M+H]⁺.

### Synthesis of (2R,4S,5R,6R)-6-((1R,2R)-1,2-dihydroxy-3-(2-(3′-hydroxy-[1,1′-biphenyl]-4-yl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 78)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(3′-acetoxy-[1,1′-biphenyl]-4-yl)acetamido)propane-1,2-diyl diacetate (6, 0.70 g, 0.791 mmol) in methanol (10 mL), was added a solution of lithium hydroxide monohydrate (0.199 g, 4.75 mmol) in water (1.0 mL). The reaction mixture was stirred at room temperature for 6 h. After completion, the reaction mixture was treated with acidic resin (Dowex 50, H⁺) until pH ˜6 and the suspension was filtered. The filtrate was concentrated, and the crude residue obtained was purified by prep HPLC using acetonitrile in water (+0.1% TFA) as eluents to afford the desired product (2R,4S,5R,6R)-6-((1R,2R)-1,2-dihydroxy-3-(2-(3′-hydroxy-[1,1′-biphenyl]-4-yl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 78). Yield: 0.024 g, 4.5%; LCMS m/z 661.40 [M+H]⁺; ¹H NMR (400 MHz, methanol-d₄) δ 7.53 (d, J=8.4 Hz, 2H), 7.36 (d, J=8.4 Hz, 2H), 7.23 (t, J=7.6 Hz, 1H), 7.06 (d, J=7.7 Hz, 1H), 7.02-7.01 (m, 1H), 6.76-6.73 (m, 1H), 4.16 (d, J=2.4 Hz, 1H), 4.01 (bs, 2H), 3.90-3.58 (m, 16 h), 3.38-3.36 (m, 1H), 3.23-3.17 (m, 1H), 2.82 (t, J=2.4 Hz, 1H), 2.76-2.71 (dd, J=4.0 Hz, 12.8 Hz, 1H), 1.75 (t, J=12.4 Hz, 1H).

### (2R,4S,5R,6R)-6-((1R,2R)-1,2-dihydroxy-3-(2-(4′-hydroxy-[1,1′-biphenyl]-4-yl)acetamido)propyl)-4-hydroxy-5-(2-hydroxy-2-methylpropanamido)-2-(2-(2-(prop-2-yn-1-yloxy)ethoxy)ethoxy)tetrahydro-2H-pyran-2-carboxylic acid (Compound 71)

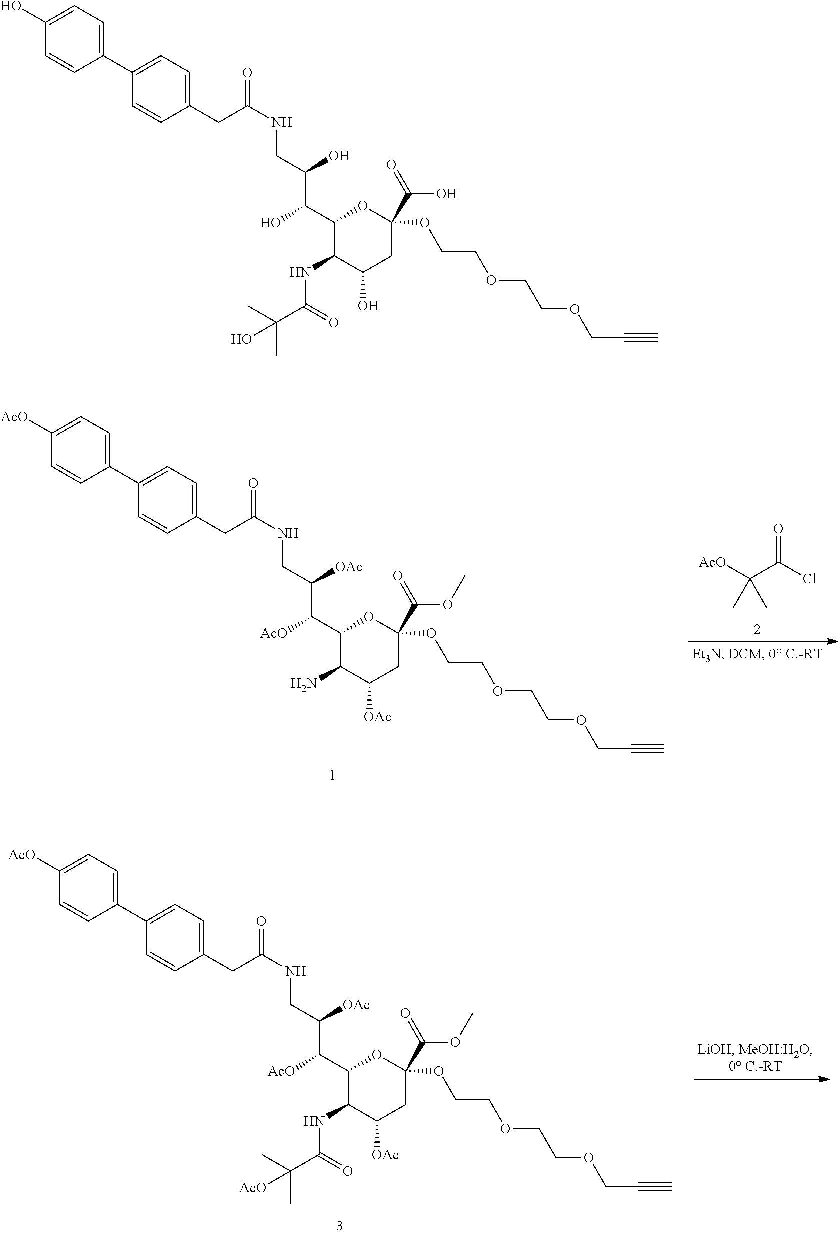

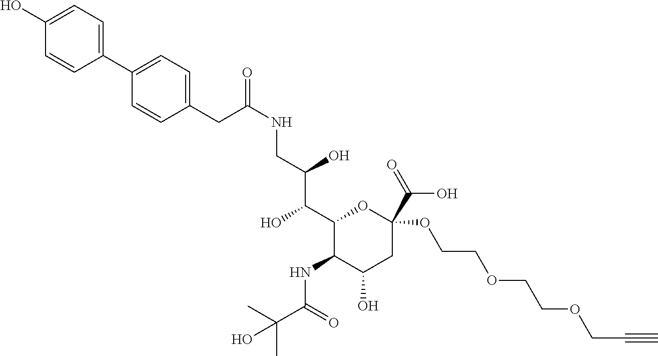 [SMILES: C#CCOCCOCCO[C@]1(C(=O)O)C[C@H](O)[C@@H](NC(=O)C(C)(C)O)[C@H]([C@H](O)[C@H](O)CNC(=O)Cc2ccc(-c3ccc(O)cc3)cc2)O1]

### Synthesis of (1R,2R)-1-((2R,3R,4S,6R)-4-acetoxy-3-(2-acetoxy-2-methylpropanamido)-6-(methoxycarbonyl)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (3)

To a solution of (1R,2R)-1-((2R,3R,4S,6R)-4-acetoxy-3-amino-6-(methoxycarbonyl)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (0.075 g, 0.0955) in tetrahydrofuran (2.0 mL), at 0° C. was added triethylamine (0.040 mL, 0.287 mmol) followed by 1-chloro-2-methyl-1-oxopropan-2-yl acetate (0.019 mL, 0.115 mmol). The reaction was stirred at 0° C. After completion, the mixture was concentrated, and the crude residue obtained was purified by silica gel column chromatography using ethyl acetate in hexane as eluents to afford the desired product (1R,2R)-1-((2R,3R,4S,6R)-4-acetoxy-3-(2-acetoxy-2-methylpropanamido)-6-(methoxycarbonyl)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (3). Yield: 0.080 g, 91%; LCMS (ESI) m/z 913.4 [M+H]⁺.

### Synthesis of (2R,4S,5R,6R)-6-((1R,2R)-1,2-dihydroxy-3-(2-(4′-hydroxy-[1,1′-biphenyl]-4-yl)acetamido)propyl)-4-hydroxy-5-(2-hydroxy-2-methylpropanamido)-2-(2-(2-(prop-2-yn-1-yloxy)ethoxy)ethoxy)tetrahydro-2H-pyran-2-carboxylic acid (Cpd. 71)

To a stirred solution of (1R,2R)-1-((2R,3R,4S,6R)-4-acetoxy-3-(2-acetoxy-2-methylpropanamido)-6-(methoxycarbonyl)-6-(2-(2-(prop-2-yn-1-yloxy)ethoxy)ethoxy) tetrahydro-2H-pyran-2-yl)-3-(2-(4′-acetoxy-[1,1′-biphenyl]-4-yl)acetamido)propane-1,2-diyl diacetate (3, 0.080 g, 0.0876 mmol) in methanol (3 mL), was added a solution of lithium hydroxide monohydrate (0.029 g, 0.701 mmol) in water (0.2 mL). The reaction mixture was stirred at room temperature for 6 h. After completion, the reaction mixture was treated with acidic resin (Dowex 50, H⁺) until pH ˜6 and the suspension was filtered. The filtrate was concentrated, and the crude residue obtained was purified by prep HPLC using acetonitrile in water (+0.1% FA) as eluents to afford the desired product (2R,4S,5R,6R)-6-((1R,2R)-1,2-dihydroxy-3-(2-(4′-hydroxy-[1,1′-biphenyl]-4-yl)acetamido)propyl)-4-hydroxy-5-(2-hydroxy-2-methylpropanamido) -2-(2-(2-(prop-2-yn-1-yloxy)ethoxy)ethoxy)tetrahydro-2H-pyran-2-carboxylic acid (Cpd. 71). Yield: 0.016 g, 26.5%; LCMS m/z 689.25 [M+H]⁺; ¹H NMR (400 MHz, methanol-d₄) δ 7.78-7.76 (m, 1H), 7.49 (d, J=8.4 Hz, 2H), 7.44-7.41 (m, 2H), 7.34 (d, J=8.4 Hz, 2H), 6.85-6.83 (m, 2H), 4.17-4.16 (m, 2H), 3.90-3.85 (m, 3H), 3.70-3.55 (m, 11H), 3.30 (bs, 1H), 2.82-2.81 (m, 1H), 2.74-2.70 (dd, J=2.4 Hz, 4.4 Hz, 1H), 1.76 (t, J=12.0 Hz, 1H), 1.37 (s, 3H), 1.36 (s, 3H).

### (2R,4S,5R,6R)-6-((1R,2R)-1,2-dihydroxy-3-(2-(4′-hydroxy-[1,1′-biphenyl]-4-yl)acetamido)propyl)-4-hydroxy-5-(3-methylureido)-2-(2-(2-(prop-2-yn-1-yloxy)ethoxy)ethoxy)tetrahydro-2H-pyran-2-carboxylic acid (Compound 70)

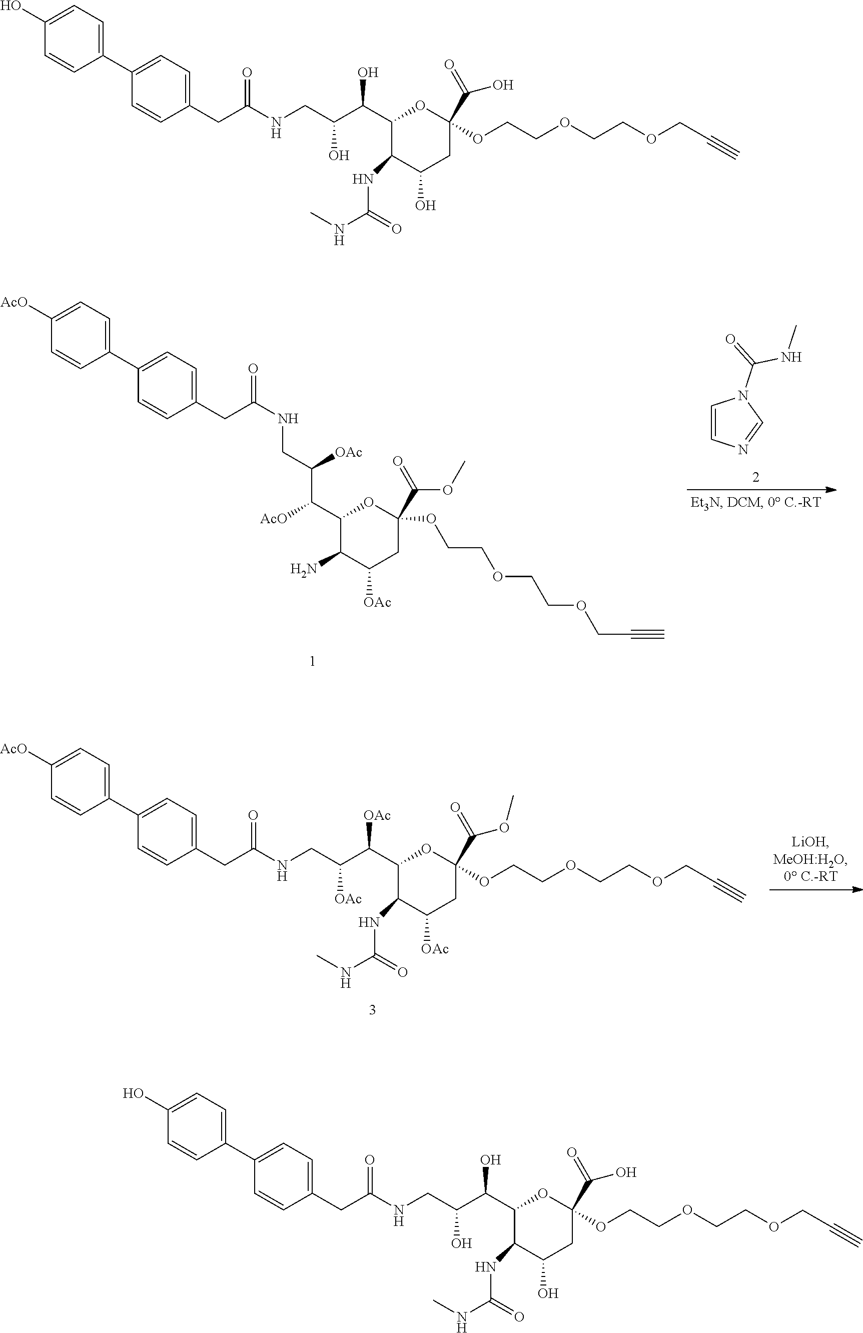

### Synthesis of (1R,2R)-1-((2R,3R,4S,6R)-4-acetoxy-6-(methoxycarbonyl)-3-(3-methylureido)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (3)

To a solution of (1R,2R)-1-((2R,3R,4S,6R)-4-acetoxy-3-amino-6-(methoxycarbonyl)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (0.10 g, 0.127) in tetrahydrofuran (2.0 mL), at 0° C. was added triethylamine (0.12 mL, 0.635 mmol) followed by N-methyl-1H-imidazole-1-carboxamide (0.032 g, 0.254 mmol). The reaction was stirred at room temperature for 16 h. After completion, the mixture was concentrated, and the crude residue obtained was purified by silica gel column chromatography using ethyl acetate in hexane as eluents to afford the desired product (1R,2R)-1-((2R,3R,4S,6R)-4-acetoxy-6-(methoxycarbonyl)-3-(3-methylureido)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (3) as a pale yellow solid. Yield: 0.10 g, 93%; LCMS (ESI) m/z 842.40 [M+H]⁺.

### Synthesis of (2R,4S,5R,6R)-6-((1R,2R)-1,2-dihydroxy-3-(2-(4′-hydroxy-[1,1′-biphenyl]-4-yl)acetamido)propyl)-4-hydroxy-5-(3-methylureido)-2-(2-(2-(prop-2-yn-1-yloxy)ethoxy)ethoxy)tetrahydro-2H-pyran-2-carboxylic acid (Cpd. 70)

To a stirred solution of (1R,2R)-1-((2R,3R,4S,6R)-4-acetoxy-6-(methoxycarbonyl)-3-(3-methylureido)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (3, 0.10 g, 0.118 mmol) in methanol (3 mL), was added a solution of lithium hydroxide monohydrate (0.030 g, 0.708 mmol) in water (0.2 mL). The reaction mixture was stirred at room temperature for 6 h. After completion, the reaction mixture was treated with acidic resin (Dowex 50, H⁺) until pH ˜6 and the suspension was filtered. The filtrate was concentrated, and the crude residue obtained was purified by prep HPLC using acetonitrile in water (+0.1% TFA) as eluents to afford the desired product (2R,4S,5R,6R)-6-((1R,2R)-1,2-dihydroxy-3-(2-(4′-hydroxy-[1,1′-biphenyl]-4-yl)acetamido)propyl)-4-hydroxy-5-(3-methylureido)-2-(2-(2-(prop-2-yn-1-yloxy)ethoxy)ethoxy)tetrahydro-2H-pyran-2-carboxylic acid (Cpd. 70). Yield: 0.008 g, 24%; LCMS m/z 660.25 [M+H]⁺; ¹H NMR (400 MHz, methanol-d₄) δ 7.50 (d, J=8.0 Hz, 2H), 7.45 (d, J=8.4 Hz, 2H), 7.33 (d, J=8.0 Hz, 2H), 6.85 (d, J=8.8 Hz, 2H), 4.17-4.16 (m, 2H), 3.89-3.88 (m, 2H), 3.65-3.45 (m, 15H), 3.31-3.24 (m, 1H), 2.82-2.81 (m, 1H), 2.74-2.70 (dd, J=2.4 Hz, 4.4 Hz, 1H), 2.67 (s, 1H), 1.74 (t, J=12.4 Hz, 1H).

### (2R,4S,5R,6R)-6-((1R,2R)-1,2-dihydroxy-3-(2-(4′-hydroxy-[1,1′-biphenyl]-4-yl)acetamido)propyl)-4-hydroxy-5-(2-mercaptoacetamido)-2-(2-(2-(prop-2-yn-1-yloxy)ethoxy)ethoxy)tetrahydro-2H-pyran-2-carboxylic acid (Compound 69)

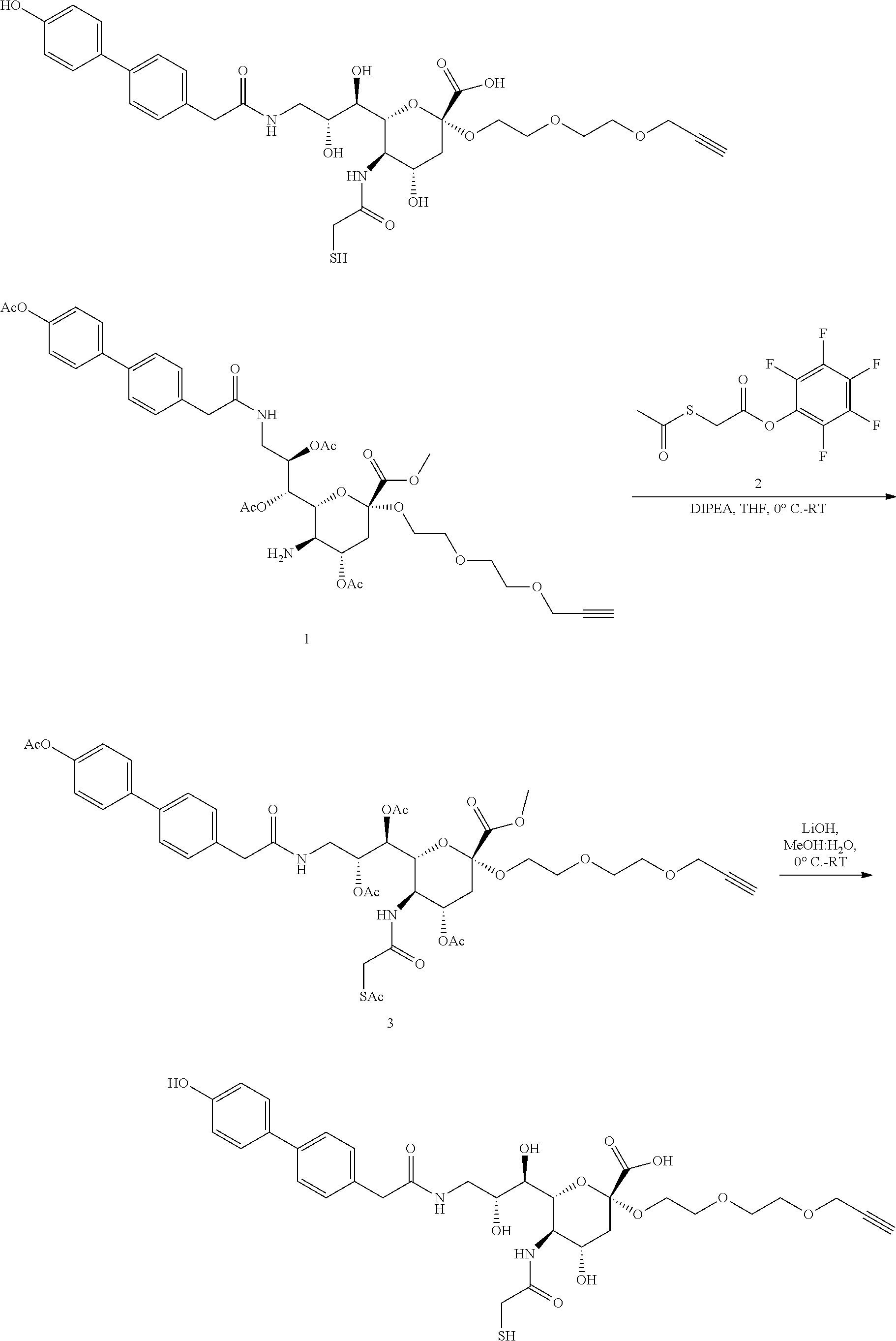

### Synthesis of (1R,2R)-1-((2R,3R,4S,6R)-4-acetoxy-3-(2-(acetylthio)acetamido)-6-(methoxycarbonyl)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (3)

To a solution of (1R,2R)-1-((2R,3R,4S,6R)-4-acetoxy-3-amino-6-(methoxycarbonyl)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (0.10 g, 0.127 mmol) in tetrahydrofuran (3.0 mL) at 0° C. was added diisopropylethylamine (0.118 mL, 0.637 mmol) followed by perfluorophenyl 2-(acetylthio)acetate (0.077 g, 0.255 mmol). The reaction was stirred at room temperature. After completion, the mixture was concentrated, and the crude residue obtained was purified by silica gel column chromatography using ethyl acetate in hexane as eluents to afford the desired product (1R,2R)-1-((2R,3R,4S,6R)-4-acetoxy-3-(2-(acetylthio)acetamido)-6-(methoxycarbonyl)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (3). Yield: 0.10 g, 72%; LCMS (ESI) m/z 901.5 [M+H]⁺.

### Synthesis of (2R,4S,5R,6R)-6-((1R,2R)-1,2-dihydroxy-3-(2-(4′-hydroxy-[1,1′-biphenyl]-4-yl)acetamido)propyl)-4-hydroxy-5-(2-mercaptoacetamido)-2-(2-(2-(prop-2-yn-1-yloxy)ethoxy)ethoxy)tetrahydro-2H-pyran-2-carboxylic acid (Cpd. 69)

To a stirred solution of (1R,2R)-1-((2R,3R,4S,6R)-4-acetoxy-3-(2-(acetylthio)acetamido)-6-(methoxycarbonyl)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (3, 0.10 g, 0.111 mmol) in methanol (3 mL), was added a solution of lithium hydroxide monohydrate (0.016 g, 0.666 mmol) in water (0.3 mL). The reaction mixture was stirred at room temperature for 6 h. After completion, the reaction mixture was treated with acidic resin (Dowex 50, H⁺) until pH ˜6 and the suspension was filtered. The filtrate was concentrated, and the crude residue obtained was purified by prep HPLC using acetonitrile in water (+0.1% FA) to afford the desired product (2R,4S,5R,6R)-6-((1R,2R)-1,2-dihydroxy-3-(2-(4′-hydroxy-[1,1′-biphenyl]-4-yl)acetamido)propyl)-4-hydroxy-5-(2-mercaptoacetamido)-2-(2-(2-(prop-2-yn-1-yloxy)ethoxy)ethoxy)tetrahydro-2H-pyran-2-carboxylic acid (Cpd. 69). Yield: 0.015 g, 20%; LCMS m/z 677.2 [M+H]⁺; ¹H NMR (400 MHz, methanol-d₄) δ 8.14-8.12 (m, 1H), 7.50 (d, J=8.4 Hz, 2H), 7.45-7.43 (m, 2H), 7.34 (d, J=8.0 Hz, 2H), 6.85 (d, J=8.4 Hz, 2H), 4.17 (d, J=2.4 Hz, 2H), 3.90-3.84 (m, 2H), 3.76-3.55 (m, 14H), 3.42-3.40 (m, 1H), 3.15 (bs, 1H), 2.83 (t, J=2.4 Hz, 1H), 2.72-2.68 (dd, J=4.0 Hz, 13.2 Hz, 1H), 1.74 (t, J=11.6 Hz, 1H).

### (2R,4S,5R,6R)-6-((1R,2R)-1,2-dihydroxy-3-(4H-thieno[3,2-c]chromene-2-carboxamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 179)

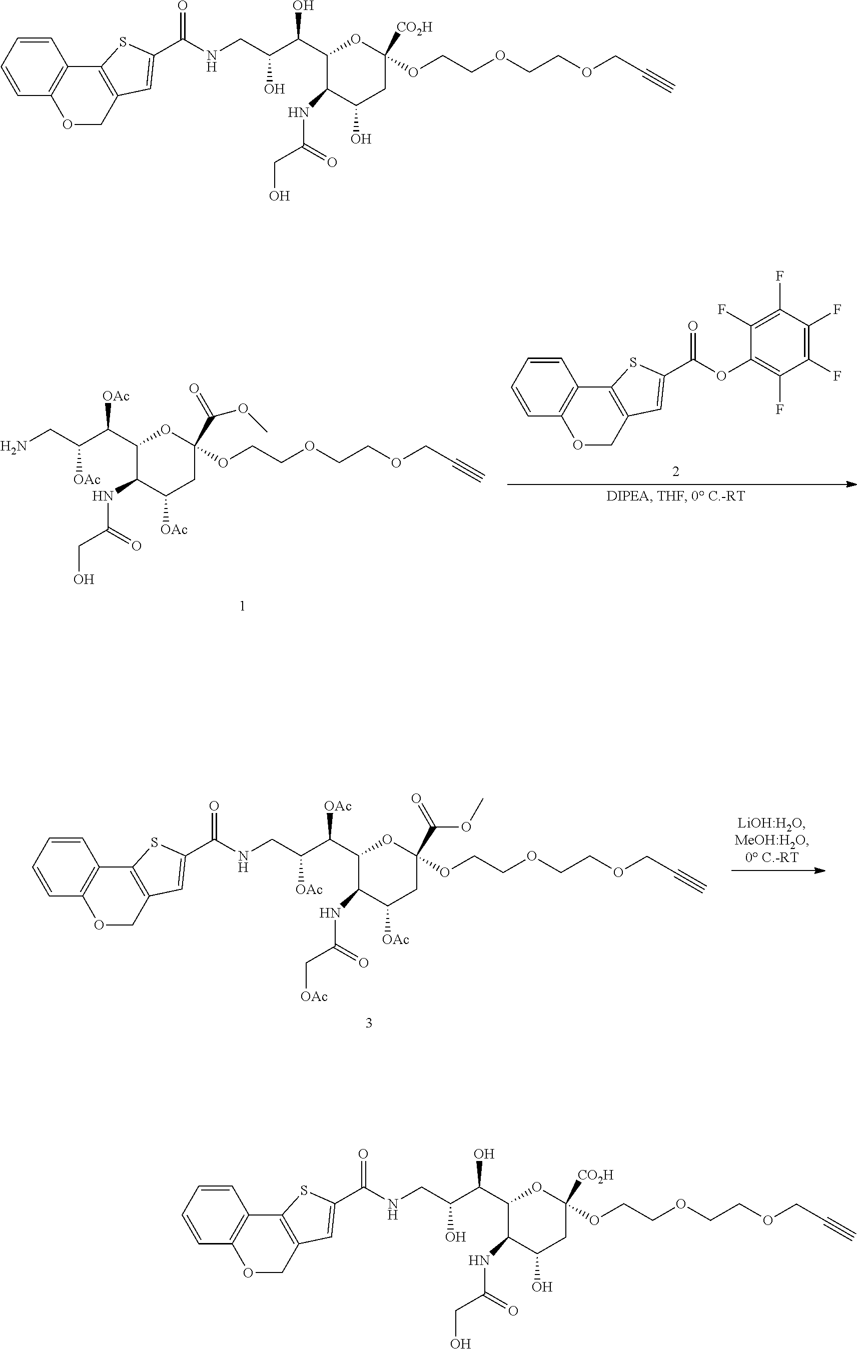

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(4H-thieno[3,2-c]chromene-2-carboxamido)propane-1,2-diyl diacetate (3)

To a solution of methyl (2R,4S,5R,6R)-6-((1R,2R)-3-amino-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylate (1, 0.250 g, 0.395 mmol), in tetrahydrofuran (5.0 mL), were added ethylbis(propan-2-yl)amine (0.36 mL, 1.98 mmol) and perfluorophenyl 4H-thieno[3,2-c]chromene-2-carboxylate (0.315 g, 0.790 mmol) at 0° C. The reaction mixture was stirred at room temperature for 6 h. After completion the mixture was concentrated, and the crude residue obtained was purified by silica gel column chromatography using ethyl acetate in hexane as eluents to afford the desired product (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(4H-thieno[3,2-c]chromene-2-carboxamido)propane-1,2-diyl diacetate (3). Yield: 0.130 g, 38%; LCMS (ESI) m/z 847.05 [M+H]⁺.

### Synthesis of (2R,4S,5R,6R)-6-((1R,2R)-1,2-dihydroxy-3-(4H-thieno[3,2-c]chromene-2-carboxamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 179)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(4H-thieno[3,2-c]chromene-2-carboxamido)propane-1,2-diyl diacetate (3, 0.130 g, 0.154 mmol) in methanol (3 mL), was added a solution of lithium hydroxide monohydrate (0.022 g, 0.921 mmol) in water (0.2 mL). The reaction mixture was stirred at room temperature for 6 h. After completion, the reaction mixture was treated with acidic resin (Dowex 50, H⁺) until pH ˜6 and the suspension was filtered. The filtrate was concentrated, and the crude residue obtained was purified by prep HPLC using acetonitrile in water (+0.1% TFA) as eluents to afford the desired product (2R,4S,5R,6R)-6-((1R,2R)-1,2-dihydroxy-3-(4H-thieno[3,2-c]chromene-2-carboxamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 179). Yield: 0.0045 g, 10%; LCMS m/z 665.35 [M+H]⁺; ¹H NMR (400 MHz, methanol-d₄) δ 7.48 (s, 1H), 7.38 (dd, J=1.2 Hz, 7.6 Hz, 1H), 7.22-7.18 (m, 1H), 6.99 (t, J=7.6 Hz, 1H), 6.92 (d, J=8.0 Hz, 1H), 5.23 (s, 2H), 4.17-4.16 (m, 2H), 4.02-3.75 (m, 8H), 3.64-3.60 (m, 7H), 3.50-3.43 (m, 2H), 2.81(t, J=2.4 Hz 1H), 2.77-2.73 (dd, J=3.6 Hz, 12.4 Hz, 1H), 1.77-1.71 (m, 1H).

### (2R,4S,5R,6R)-6-((1R,2R)-1,2-dihydroxy-3-propionamidopropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 15)

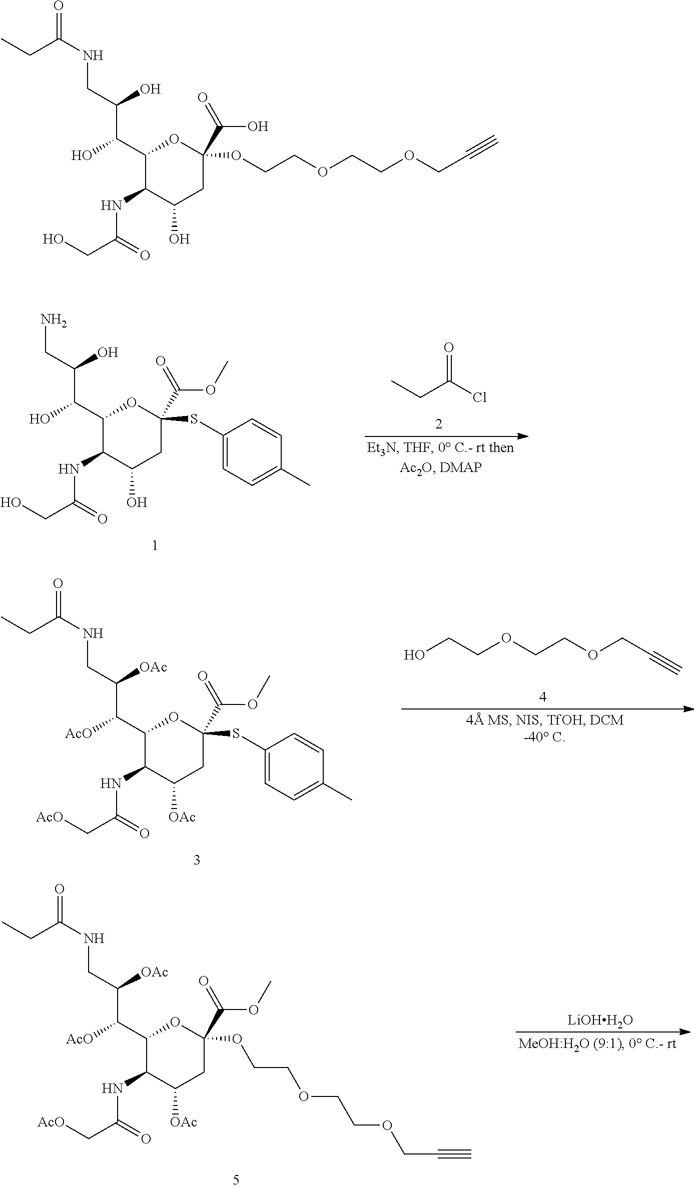

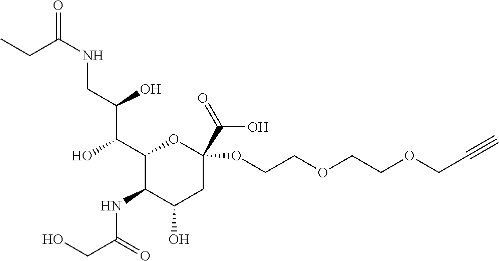 [SMILES: C#CCOCCOCCO[C@]1(C(=O)O)C[C@H](O)[C@@H](NC(=O)CO)[C@H]([C@H](O)[C@H](O)CNC(=O)CC)O1]

### Synthesis of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-propionamidopropane-1,2-diyl diacetate (3)

To a solution of methyl (2R,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (1, 0.500 g, 1.12 mmol) in tetrahydrofuran (5 mL), was added triethylamine (0.47 mL, 3.36 mmol) at 0° C. followed by propionyl chloride (2, 0.097 mL, 1.12 mmol) dropwise. The mixture was allowed to stir at room temperature for 3 h. After completion, acetic anhydride (0.53 mL, 5.60 mmol) and 4-dimethylaminopyrdine (0.5 g, 4.09 mmol) mixture was concentrated. The reaction mixture was diluted with ethyl acetate (50 mL), washed once with water, and twice with brine. The organic layer was dried, filtered, concentrated, and purified by silica gel column chromatography using ethyl acetate in hexane as eluents to afford the desired product (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-propionamidopropane-1,2-diyl diacetate (3). Yield: 0.450 g, 60%; LCMS (ESI) m/z 669.9 [M+H]⁺.

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-propionamidopropane-1,2-diyl diacetate (5)

A mixture of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-propionamidopropane-1,2-diyl diacetate (3, 0.450 g, 0.67 mmol), 2-[2-(prop-2-yn-1-yloxy)ethoxy]ethan-1-ol (4, 0.485 g, 3.35 mmol) and activated 4 Å powdered molecular sieves (0.5 g) in anhydrous dichloromethane (10 mL) was stirred at room temperature for 15 h then cooled to −40° C. 1-Iodopyrrolidine-2,5-dione (0.378 g, 1.68 mmol) and trifluoromethanesulfonic acid (0.006 mL, 0.07 mmol) in dichloromethane (1.0 mL) were added and the mixture was stirred at this temperature for 1 h. After completion, the reaction mixture was quenched by addition of triethyl amine (0.5 mL) and warmed up to room temperature. The reaction mixture was filtered, and the filtrate was washed with aqueous saturated solution of sodium bicarbonate, dried, filtered, and concentrated. The crude residue obtained was purified by silica gel column chromatography using ethyl acetate in hexane as eluents to afford the desired product (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-propionamidopropane-1,2-diyl diacetate (5). Yield: 0.41 g, 86%; LCMS (ESI) m/z 689.9 [M+H]⁺.

### Synthesis of (2R,4S,5R,6R)-6-((1R,2R)-1,2-dihydroxy-3-propionamidopropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 15)

To a solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-propionamidopropane-1,2-diyl diacetate (5, 0.400 g, 0.581 mmol) in methanol (10.0 mL), was added a solution of LiOH·H₂O (0.143 g, 3.49 mmol) in water (1 mL). The reaction mixture was stirred at room temperature for 6 h. After completion, the reaction mixture was treated with acidic resin (Dowex 50, H⁺) until pH ˜7 and the suspension was filtered, and the filtrate was concentrated, and the crude residue obtained was purified by Prep-HPLC acetonitrile in water (+0.1% TFA) as eluents to afford the desired product (2R,4S,5R,6R)-6-((1R,2R)-1,2-dihydroxy-3-propionamidopropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 15). Yield: 0.019 g, 26%; LCMS m/z 507.00 [M+H]⁺; 1H NMR (400 MHz, Methanol-d4) δ 4.19 (d, J=2.4 Hz, 2H), 4.02 (s, 2H), 3.94-3.79 (m, 4H), 3.75-3.73 (m, 1H), 3.69-3.59 (m, 8H), 3.37 (dd, J=8.8 & 1.6 Hz, 1H), 3.27-3.22 (m, 1H), 2.84 (t, J=2.4 Hz, 2.4 Hz, 1H), 2.73 (dd, J=13.2 & 4.0 Hz, 1H), 2.23 (q, J=7.6 Hz, 2H), 1.75 (t, J=11.6 Hz, 12.4 Hz, 1H), 1.13 (t, J=7.6 Hz, 7.6 Hz, 3H).

### (2R,4S,5R,6R)-6-((1R,2R)-3-(4-chlorobenzamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 30)

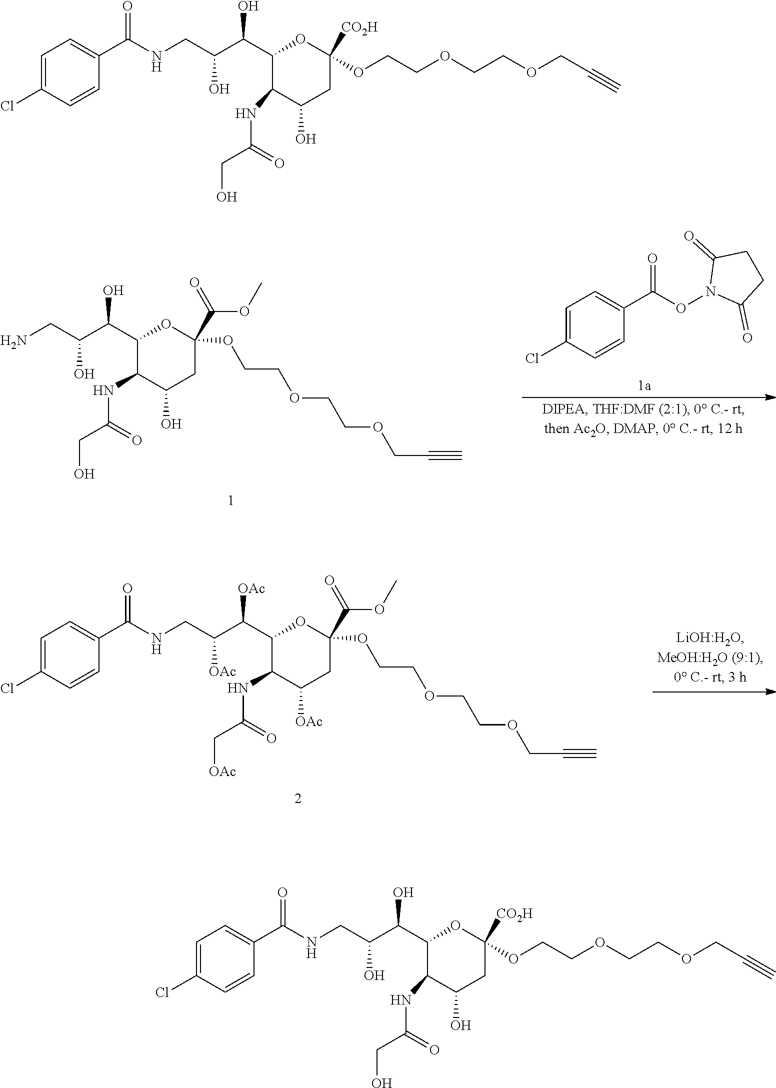

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(4-chlorobenzamido)propane-1,2-diyl diacetate (2)

To a stirred solution of methyl (2R,4S,5R,6R)-6-((1R,2R)-3-amino-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylate (1, 0.50 g, 1.08 mmol) and 2,5-dioxopyrrolidin-1-yl 4-chlorobenzoate (1a, 0.218 g, 0.861 mmol) in tetrahydrofuran (10.0 mL) was added ethylbis(propan-2-yl)amine (0.938 mL 5.38 mmol) at 0° C. then stirred at room temperature for 4 h. Acetic anhydride (0.627 mL, 22.50 mmol) and N, N-dimethyl aniline (0.05 g, 0.332 mmol) were added at 0° C. and the resulting mixture was stirred at room temperature for 12 h. After completion the mixture was diluted with water and ethyl acetate. The organic layer was separated, dried, filtered, concentrated, and the crude residue obtained was purified by silica gel column chromatography using ethyl acetate in hexane as eluents to afford the desired product (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(4-chlorobenzamido)propane-1,2-diyl diacetate (2). Yield: 0.200 g, 24%; LCMS (ESI) m/z 771.62 [M+H]⁺.

### Synthesis of (2R,4S,5R,6R)-6-((1R,2R)-3-(4-chlorobenzamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 30)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(4-chlorobenzamido)propane-1,2-diyl diacetate (2, 0.200 g, 0.259 mmol) in methanol (10.0 mL), was added a solution of lithium hydroxide monohydrate (0.065 g, 1.56 mmol) in water (0.5 mL). The reaction mixture was stirred at room temperature for 6 h. After completion, the reaction mixture was treated with acidic resin (Dowex 50H⁺) up to pH ˜6 and the suspension was filtered. The filtrate was concentrated, and the crude residue obtained was purified by prep HPLC using acetonitrile in water (+0.1% TFA) as eluents to afford the desired product (2R,4S,5R,6R)-6-((1R,2R)-3-(4-chlorobenzamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 30). Yield: 0.025 g 16.37%; LCMS m/z 589.17 [M+H]⁺; 1H NMR (400 MHz, methanol-d4) δ 7.87 (s, J=7.6 Hz, 1H), 7.81 (d, J=2.0 Hz, 2H), 7.4 (d, J=8.4 Hz, 2H), 4.1 (d, J=2.4 Hz, 2H) 3.96-3.94 (m, 3H), 3.94-3.86 (m, 1H), 3.86-3.82 (m, 2H), 3.82-3.78 (m 2H),3.65-3.58 (m, 7H), 3.52-3.49, (m, 1H), 3.48-3.43 (m, 1H), 2.82 (t, J=4.8 Hz 1H), 2.7 (dd, J=18.4 Hz, 2H), 1.7 (m, 1H).

### (2R,4S,5R,6R)-6-((1R,2R)-1,2-dihydroxy-3-(2-(4-hydroxyphenyl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 153)

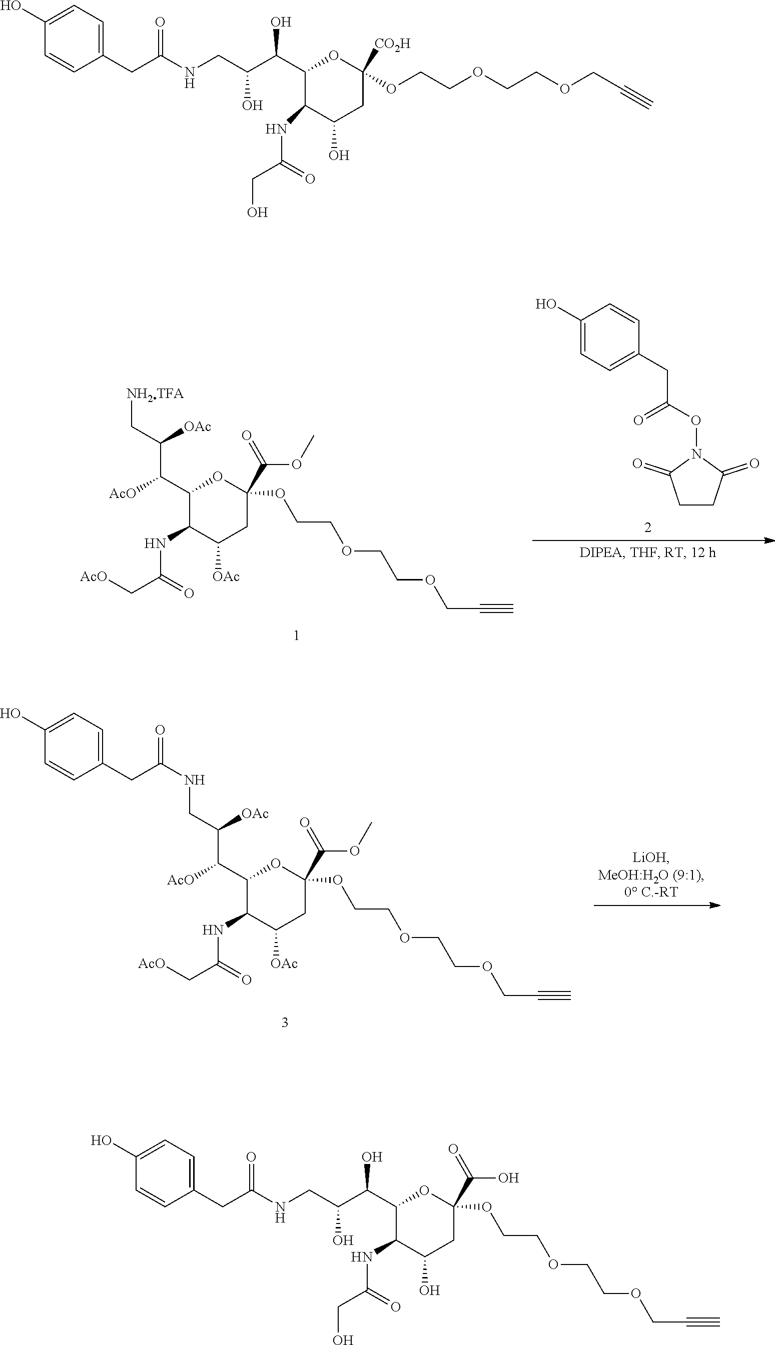

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(4-hydroxyphenyl)acetamido)propane-1,2-diyl diacetate (3)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-aminopropane-1,2-diyl diacetate (1, 0.150 g, 0.24 mmol) and 2,5-dioxopyrrolidin-1-yl 2-(4-hydroxyphenyl)acetate (2, 0.070 g, 0.29 mmol) in tetrahydrofuran (3.0 mL), was added ethylbis(propan-2-yl)amine (0.207 mL, 1.19 mmol) at 0° C. then stirred at room temperature for 12 h. After completion the mixture was diluted with water and ethyl acetate. The organic layer was separated, dried, filtered, concentrated, and the crude residue obtained was purified by silica gel column chromatography using ethyl acetate in hexane as eluents to afford the desired product (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(4-hydroxyphenyl)acetamido)propane-1,2-diyl diacetate (3). Yield: 0.090 g, 49%; LCMS (ESI) m/z 766.95 [M+H]⁺.

### Synthesis of (2R,4S,5R,6R)-6-((1R,2R)-1,2-dihydroxy-3-(2-(4-hydroxyphenyl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 153)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(4-hydroxyphenyl)acetamido)propane-1,2-diyl diacetate (2, 0.09 g, 0.12 mmol) in methanol (3.0 mL), was added a solution of lithium hydroxide monohydrate (0.029 g, 0.71 mmol) in water (0.30 mL). The reaction mixture was stirred at room temperature for 6 h. After completion, the reaction mixture was treated with acidic resin (Dowex 50H⁺) up to pH ˜6 and the suspension was filtered. The filtrate was concentrated, and the crude residue obtained was purified by prep HPLC using acetonitrile in water (+0.1% TFA) as eluents to afford the desired product (2R,4S,5R,6R)-6-((1R,2R)-1,2-dihydroxy-3-(2-(4-hydroxyphenyl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 153). Yield: 0.007 g, 10%; LCMS m/z 585.35 [M+H]+; ¹H NMR (400 MHz, methanol-d4) δ 7.85 (d, J=7.2 Hz, 1H), 7.72 (s, 1H), 7.10 (d, J=8.4 Hz, 2H), 6.72 (d, J=8.4 Hz, 2H), 4.18 (d, J=2.4 Hz, 1H), 4.03 (s, 1H), 3.93-3.73 (m, 4H), 3.72-3.57 (m, 9H), 3.43 (s, 2H), 3.43 (s, 2H), 3.35-3.31 (m, 1H), 3.22-3.17 (m, 1H), 2.83 (t, J=2.4 Hz, 1H), 2.74 (dd, J=12.4 & 3.6 Hz, 1H), 1.72 (t, J=12.0 Hz, 1H).

### (2R,4S,5R,6R)-6-((1R,2R)-1,2-dihydroxy-3-(2-(3-hydroxyphenyl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 152)

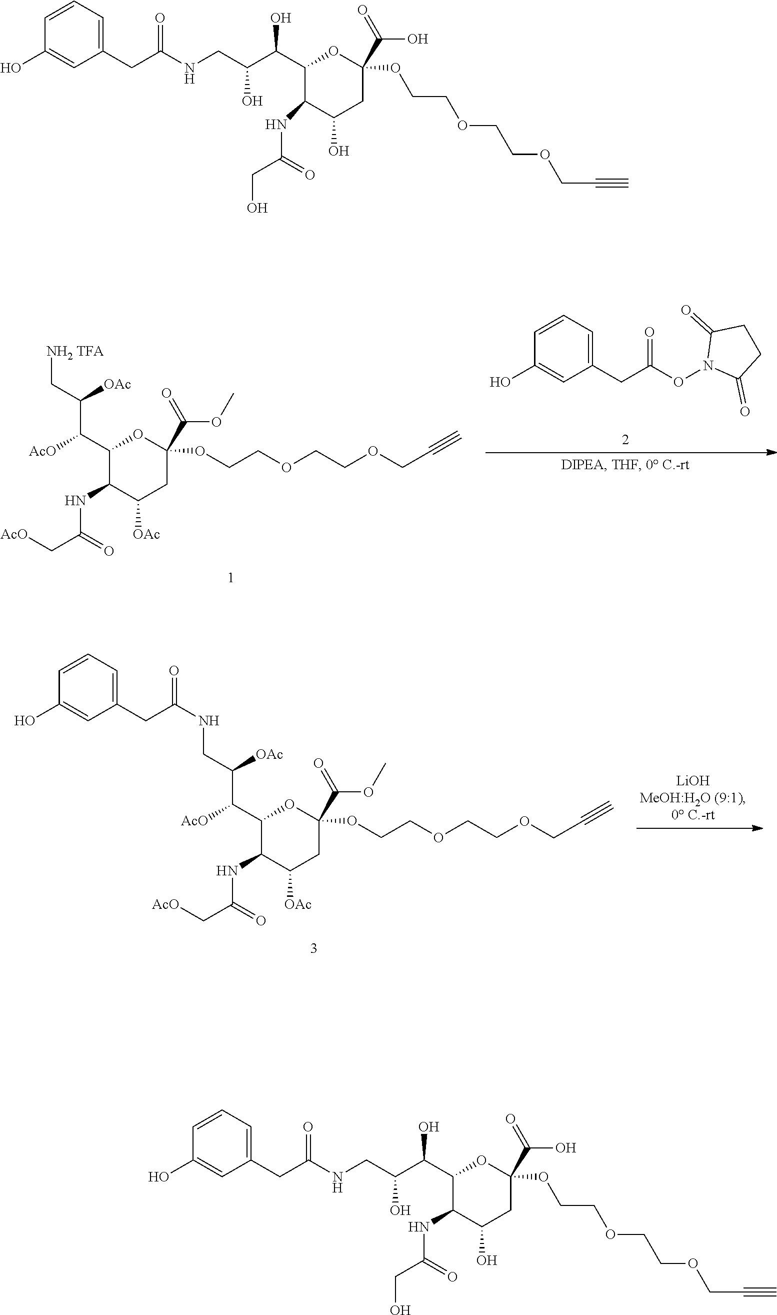

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(3-hydroxyphenyl)acetamido)propane-1,2-diyl diacetate (3)

To a solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-aminopropane-1,2-diyl diacetate (1, 0.400 g, 0.55 mmol), in tetrahydrofuran (10.0 mL), were added ethylbis(propan-2-yl)amine (0.47 mL, 2.74 mmol) and 2,5-dioxopyrrolidin-1-yl 2-(3-hydroxyphenyl)acetate (2, 0.109 g, 0.44 mmol) at 0° C. then stirred at room temperature for 6 h. After completion the mixture was concentrated, and the crude residue obtained was purified by silica gel column chromatography using ethyl acetate in hexane as eluents to afford the desired product (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(3-hydroxyphenyl)acetamido)propane-1,2-diyl diacetate (3). Yield: 0.230 g, 52%; LCMS (ESI) m/z 766.95 [M+H]⁺.

### Synthesis of (2R,4S,5R,6R)-6-((1R,2R)-1,2-dihydroxy-3-(2-(3-hydroxyphenyl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 152)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(3-hydroxyphenyl)acetamido)propane-1,2-diyl diacetate (3, 0.230 g, 0.30 mmol) in methanol (5 mL), was added a solution of lithium hydroxide monohydrate (0.073 g, 1.78 mmol) in water (0.5 mL). The reaction mixture was stirred at room temperature for 6 h. After completion, the reaction mixture was treated with acidic resin (Dowex 50, H⁺) up to pH ˜6 and the suspension was filtered. The filtrate was concentrated, and the crude residue obtained was purified by prep HPLC using acetonitrile in water (+0.1% TFA) as eluents to afford the desired product (2R,4S,5R,6R)-6-((1R,2R)-1,2-dihydroxy-3-(2-(3-hydroxyphenyl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 152). Yield: 29 mg, 16%; LCMS m/z 585.25 [M+H]⁺; ¹H NMR (400 MHz, methanol-d₄) δ 7.11 (t, J=7.6 Hz, 8.0 Hz, 1H), 6.76-6.65 (m, 3H), 4.19 (d, J=2.4 Hz, 2H), 4.02 (s, 2H), 3.93-3.85 (m, 2H), 3.83-3.71 (m, 3H), 3.67-3.63 (m, 8H), 3.47 (brs, 2H), 3.35-3.32 (m, 1H), 3.24-3.19 (m, 1H), 2.84 (t, J=3.0 Hz, 3.0 Hz, 1H), 2.80 (dd, J=12.4 & 4.0 Hz, 1H), 1.65 (t, J=12.4 Hz, 12.4 Hz, 1H).

### (2R,4S,5R,6R)-6-((1R,2R)-1,2-dihydroxy-3-(2-((1r,4R)-4-hydroxycyclohexyl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 14)

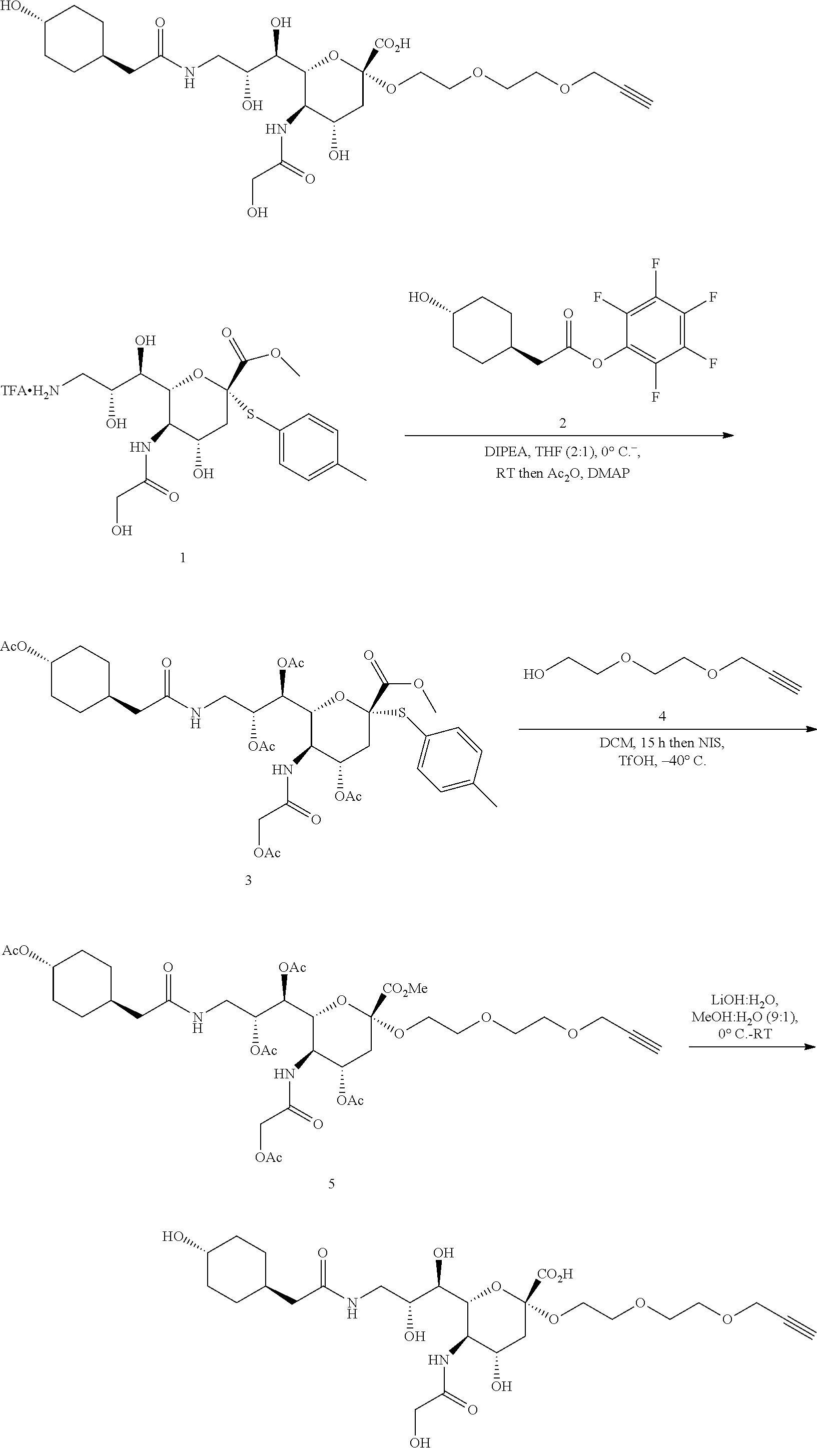

### Synthesis of (1R,2R)-1-((2R,3R,4S,6S)-4-acetoxy-3-(2-acetoxyacetamido)-6-(methoxycarbonyl)-6-(p-tolylthio)tetrahydro-2H-pyran-2-yl)-3-(2-((1r,4R)-4-acetoxycyclohexyl)acetamido)propane-1,2-diyl diacetate (3)

To a solution of methyl (2R,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (1, 0.5 g, 1.12 mmol) and perfluorophenyl 2-((1r,4r)-4-hydroxycyclohexyl)acetate (2, 0.291 g, 0.90 mmol) in tetrahydrofuran (5 mL), was added N,N-Diisopropyl ethyl amine (1.58 mL, 11.25 mmol) at 0° C. then stirred at room temperature for 3 h. After completion, acetic anhydride (1.0 mL, 11.2 mmol) and 4-dimethylaminopyrdine (0.136 g, 1.12 mmol) were sequentially added at 0° C. and the resulting mixture was stirred at room temperature for 12 h. After completion, the reaction mixture was diluted with ethyl acetate and washed with water. The organic layer was separated, dried, filtered, and concentrated. The crude residue was purified by silica gel column chromatography using ethyl acetate in hexane as eluents to afford the desired product (1R,2R)-1-((2R,3R,4S,6S)-4-acetoxy-3-(2-acetoxyacetamido)-6-(methoxycarbonyl)-6-(p-tolylthio)tetrahydro-2H-pyran-2-yl)-3-(2-((1r,4R)-4-acetoxycyclohexyl)acetamido)propane-1,2-diyl diacetate (3). Yield: 0.80 g, 89.47%; ELSD-MS m/z 795.00 [M+H]⁺.

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-((1r,4R)-4-acetoxycyclohexyl)acetamido)propane-1,2-diyl diacetate (5)

A mixture of (1R,2R)-1-((2R,3R,4S,6S)-4-acetoxy-3-(2-acetoxyacetamido)-6-(methoxycarbonyl)-6-(p-tolylthio)tetrahydro-2H-pyran-2-yl)-3-(2-((1r,4R)-4-acetoxycyclohexyl)acetamido)propane-1,2-diyl diacetate (3, 0.450 g, 0.67 mmol), 2-[2-(prop-2-yn-1-yloxy)ethoxy]ethan-1-ol (4, 0.291 g, 2.01 mmol) and activated 4 Å powdered molecular sieves (0.5 g) in anhydrous dichloromethane (10 mL) was stirred at room temperature for 15 h then cooled down to −40° C. 1-Iodopyrrolidine-2,5-dione (0.227 g, 1.00 mmol) and trifluoromethanesulfonic acid (0.005 mL, 0.067 mmol) in dichloromethane (1.0 mL) were added at −40° C. and the resulting mixture was stirred for 1 h at this temperature. After completion, the reaction mixture was quenched by addition of triethyl amine (0.5 mL) and warmed up to room temperature. The reaction mixture was filtered, and the filtrate was washed with aqueous saturated solution of sodium bicarbonate. The organic layer was dried, filtered, and concentrated. The crude residue obtained was purified by silica gel column chromatography using, ethyl acetate in hexane as eluents to afford the desired product (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-((1r,4R)-4-acetoxycyclohexyl)acetamido)propane-1,2-diyl diacetate (5) as a mixture of anomers. Yield: 0.4 g, 86.68%; ELSD-MS m/z 812.80 [M−H]⁻.

### Synthesis of (2R,4S,5R,6R)-6-((1R,2R)-1,2-dihydroxy-3-(2-((1r,4R)-4-hydroxycyclohexyl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 14)

To a solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-propionamidopropane-1,2-diyl diacetate (5, 0.268 g, 0.389 mmol) in methanol (10.0 mL), was added a solution of LiOH·H2O (0.060 g, 2.33 mmol) in water (1 mL). The reaction mixture was stirred at room temperature for 6 h. After completion, the reaction mixture was treated with acidic resin (Dowex 50, H⁺) up to PH ˜7 and the suspension was filtered. The filtrate was concentrated, and the crude residue obtained was purified by Prep-HPLC using acetonitrile in water (+0.1% FA) as eluents to afford the desired product (2R,4S,5R,6R)-6-((1R,2R)-1,2-dihydroxy-3-(2-((1r,4R)-4-hydroxycyclohexyl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 14). Yield: 0.040 g, 20.59%; LCMS m/z 507.00 [M+H]⁺; ¹H-NMR (400 MHz, methanol-d₄) δ 4.19 (d, J=2.4 Hz, 2H), 4.03 (brs, 2H), 3.93-3.78 (m, 3H), 3.73-3.71 (m, 1H), 3.68-3.56 (m, 8H), 3.53-3.42 (m, 2H), 3.37-3.35 (m, 1H), 3.32-3.30 (m, 1H), 2.84 (t, J=2.0 Hz, 2.4 Hz, 1H), 2.78 (dd, J=12.8 & 4.8 Hz, 1H), 2.10 (d, J=7.6 Hz, 2H), 1.92 (dd, J=12.8 & 4.8 Hz, 2H), 1.77-1.65 (m, 4H), 1.30-1.20 (m, 2H), 1.09-1.00 (m, 2H).

### (2R,4S,5R,6R)-6-((1R,2R)-3-(2-(4-chlorophenyl)acetamido)-1,2-dihydroxypropyl)-4-hydroxy-5-(2-methoxyacetamido)-2-((6-(prop-2-yn-1-yloxy)hexyl)oxy)tetrahydro-2H-pyran-2-carboxylic acid (Compound 192)

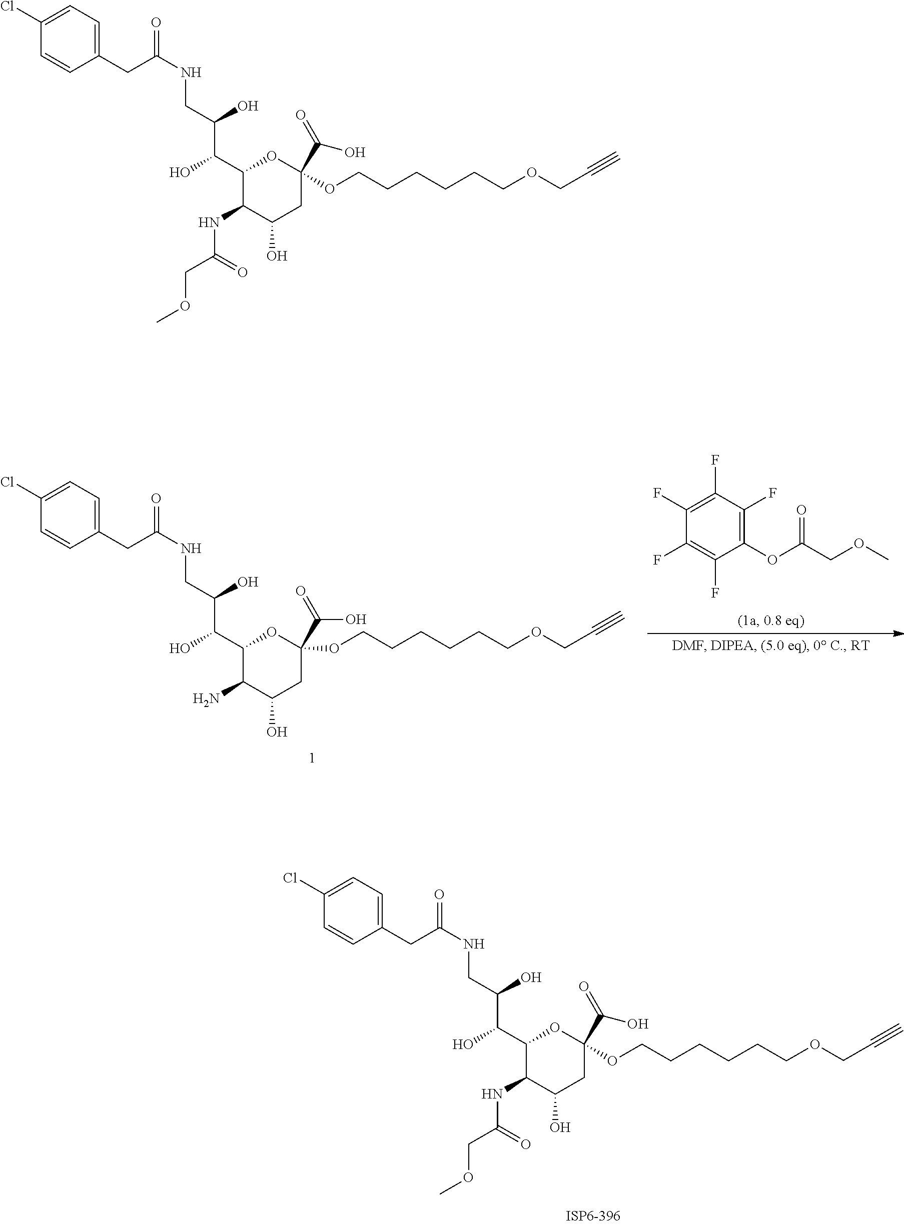 [SMILES: C#CCOCCCCCCO[C@]1(C(=O)O)C[C@H](O)[C@@H](NC(=O)COC)[C@H]([C@H](O)[C@H](O)CNC(=O)Cc2ccc(Cl)cc2)O1 ; C#CCOCCCCCCO[C@]1(C(=O)O)C[C@H](O)[C@@H](N)[C@H]([C@H](O)[C@H](O)CNC(=O)Cc2ccc(Cl)cc2)O1 ; COCC(=O)Oc1c(F)c(F)c(F)c(F)c1F ; C#CCOCCCCCCO[C@]1(C(=O)O)C[C@H](O)[C@@H](NC(=O)COC)[C@H]([C@H](O)[C@H](O)CNC(=O)Cc2ccc(Cl)cc2)O1]

### Synthesis of (2R,4S,5R,6R)-6-((1R,2R)-3-(2-(4-chlorophenyl)acetamido)-1,2-dihydroxypropyl)-4-hydroxy-5-(2-methoxyacetamido)-2-((6-(prop-2-yn-1-yloxy)hexyl)oxy)tetrahydro-2H-pyran-2-carboxylic acid (Cpd. 192)

To a stirred solution of (2R,4S,5R,6R)-5-amino-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (1, 0.12 g, 1.0 eq., 0.21 mmol) and perfluorophenyl 2-methoxyacetate (1a, 0.044 g, 0.8 eq., 0.18 mmol) in dimethylformamide (2.0 ml) was added ethylbis(propan-2-yl)amine (0.18 ml, 5.0 eq., 1.05 mmol) at 0° C. and the reaction mixture was stirred at room temperature for 2 h. After completion, the reaction mixture was concentrated under reduced pressure and the crude residue obtained was purified by prep HPLC using acetonitrile and water (+0.1% TFA) as eluents to afford the desired product (2R,4S,5R,6R)-6-((1R,2R)-3-(2-(4-chlorophenyl)acetamido)-1,2-dihydroxypropyl)-4-hydroxy-5-(2-methoxyacetamido)-2-((6-(prop-2-yn-1-yloxy)hexyl)oxy)tetrahydro-2H-pyran-2-carboxylic acid (Cpd. 192) as an off white solid. Yield: 0.045 g, 33%; LCMS m/z 629.1 [M+H]+; 1H NMR (400 MHz, Methanol-d4): δ 7.86 (d, J=8.0 Hz, 1H), 7.32-7.28 (m, 4H), 4.12 (d, J=2.4 Hz, 2H), 3.95-3.68 (m, 6H), 3.60-3.50 (m, 4H), 3.42-3.35 (m, 5H), 3.22-3.18 (m, 1H), 2.81 (t, J=2.4 Hz, 1H), 2.70 (dd, J=12.4 Hz & 4.0 Hz, 1H), 1.73 (t, J=12.4 Hz, 3H), 1.61-1.52 (m, 4H), 1.40-1.33 (m, 4H).

### (2R,4S,5R,6R)-6-((1R,2R)-3-benzamido-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 202)

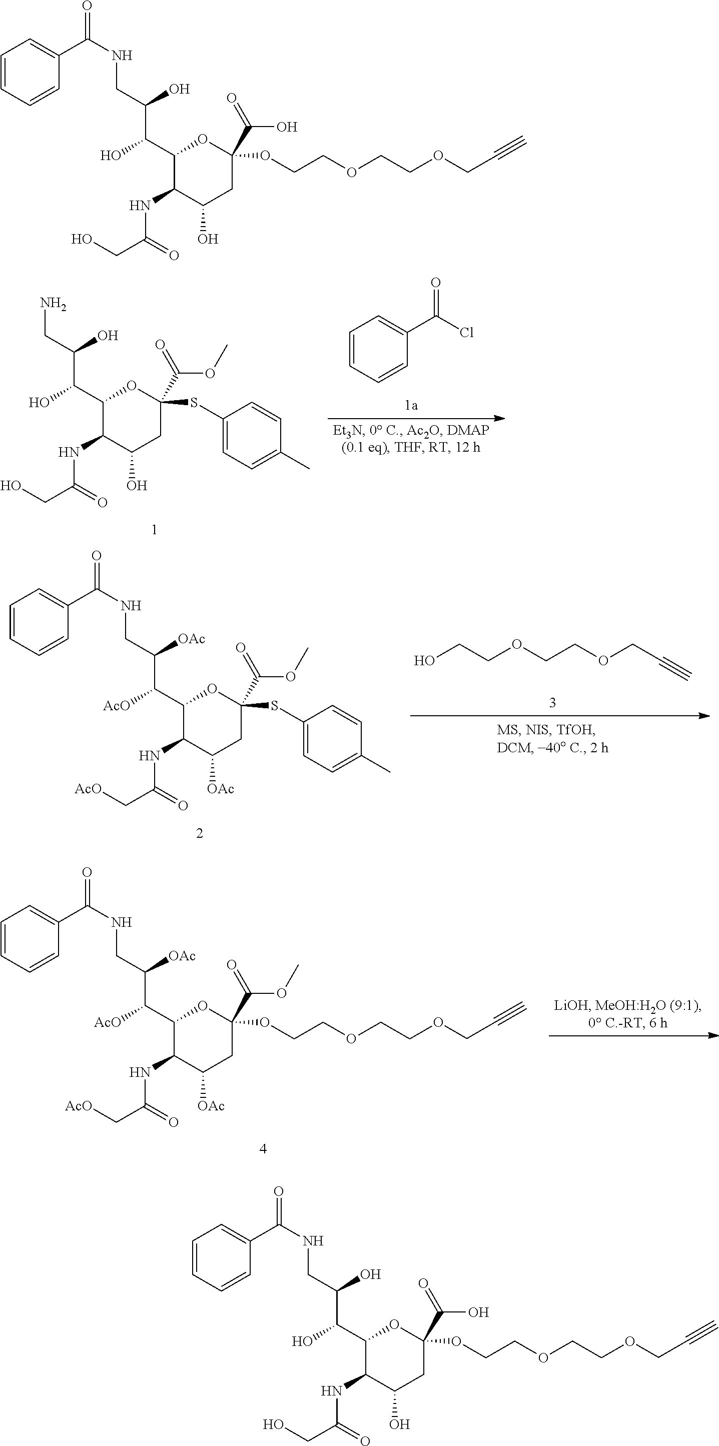

### Synthesis of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-benzamidopropane-1,2-diyl diacetate (2)

To a stirred solution of methyl (2R,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (1, 0.150 g, 0.24 mmol) and benzoyl chloride (1a, 0.070 g, 0.29 mmol) in tetrahydrofuran (10.0 mL), was added triethylamine (2.53 mL, 18.00 mmol) at 0° C. then stirred at room temperature for 3 h. Acetic anhydride (1.70 mL, 18.00 mmol) and N, N-dimethyl aniline (0.110 g, 0.900 mmol) were added at 0° C. and the resulting mixture was stirred at room temperature for 12 h. After completion the mixture was diluted with water and ethyl acetate. The organic layer was separated, dried, filtered, and concentrated. The crude residue obtained was purified by silica gel column chromatography using ethyl acetate in hexane as eluents to afford the desired product (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-benzamidopropane-1,2-diyl diacetate (2). Yield: 0.500 g, 39%; LCMS (ESI) m/z 716.90 [M+H]⁺.

### Synthesis of (1R,2R)-1-((2R,3R,4S,6R)-4-acetoxy-3-(2-acetoxyacetamido)-6-((4-(but-3-yn-1-yloxy)benzyl)oxy)-6-(methoxycarbonyl)tetrahydro-2H-pyran-2-yl)-3-(2-(4′-acetoxy-[1,1′-biphenyl]-4-yl)acetamido)propane-1,2-diyl diacetate (4)

To a mixture of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-benzamidopropane-1,2-diyl diacetate (2, 0.30 g, 0.42 mmol) and 2-(2-(prop-2-yn-1-yloxy)ethoxy)ethan-1-ol (3, 0.181 g, 1.26 mmol) and activated 4 Å powdered molecular sieves (1.00 g) in anhydrous dichloromethane (10.0 ml) at room temperature for 15 h then cooled down to −40° C. 1-Iodopyrrolidine-2,5-dione (0.235 g, 1.05 mmol) and trifluoromethanesulfonic acid (0.036 mL, 0.42 mmol) in dichloromethane (1.0 mL) were added at −40° C. and the resulting mixture was stirred for 1 h at this temperature. After completion, the reaction mixture was quenched by addition of triethyl amine (1.0 mL) and warmed up to room temperature. The reaction mixture was filtered, and the filtrate was washed with a saturated solution of sodium bicarbonate and dried, filtered, and concentrated. The crude residue obtained was purified by silica gel column chromatography using ethyl acetate in hexane as eluents to afford the desired product (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-benzamidopropane-1,2-diyl diacetate (3). Yield: 0.190 g, 62%; LCMS m/z 734.90 [M−H]⁻.

### Synthesis of (2R,4S,5R,6R)-6-((1R,2R)-3-benzamido-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 202)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-benzamidopropane-1,2-diyl diacetate (3, 0.190 g, 0.26 mmol) in methanol (3.0 mL), was added a solution of lithium hydroxide monohydrate (0.066 g, 1.55 mmol) in water (0.30 mL). The reaction mixture was stirred at room temperature for 6 h. After completion, the reaction mixture was treated with acidic resin (Dowex 50H⁺) up to pH ˜6 and the suspension was filtered. The filtrate was concentrated, and the crude residue obtained was purified by prep HPLC using acetonitrile in water (+0.1% TFA) as eluents to afford the desired product (2R,4S,5R,6R)-6-((1R,2R)-3-benzamido-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 202). Yield: 0.0035 g, 3%; LCMS m/z 555.30 [M+H]+; ¹H NMR (400 MHz, methanol-d4) δ 7.83 (d, J=7.2 Hz, 2H), 7.54-7.43 (m, 3H), 4.16 (d, J=2.4 Hz, 2H), 4.05-3.97 (m, 3H), 3.95-3.91 (m, 1H), 3.85-3.76 (m, 4H), 3.64-3.62 (m, 7H), 3.54-3.44 (m, 2H), 2.81 (t, J=2.4 Hz, 1H), 2.75 (dd, J=12.8 & 4.0 Hz, 1H), 1.75 (t, J=12.0 Hz, 1H).

### (2R,4S,5R,6R)-6-((1R,2R)-3-(3-chlorobenzamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 201)

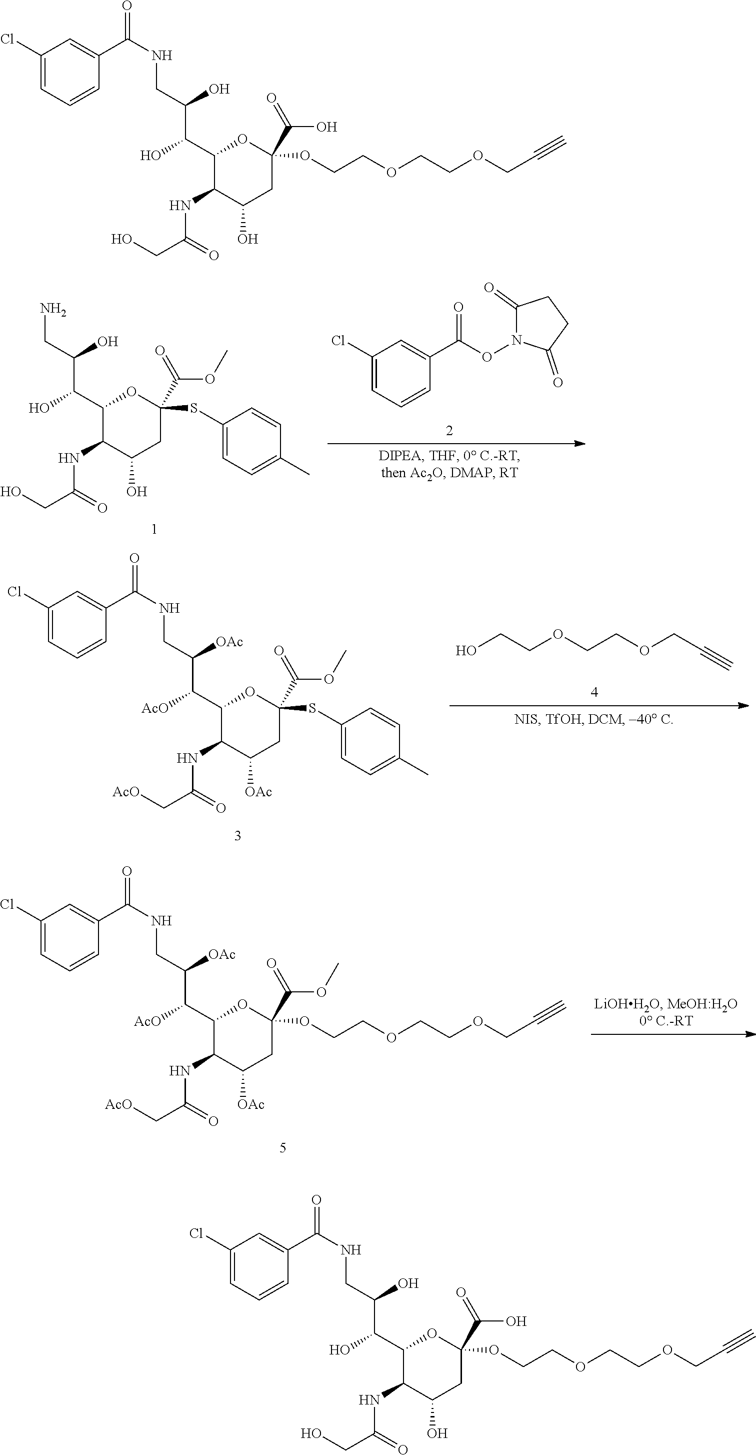

### Synthesis of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(3-chlorobenzamido)propane-1,2-diyl diacetate (3)

Methyl (2R,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (1, 0.70 g, 1.57 mmol) and 2,5-dioxopyrrolidin-1-yl 3-chlorobenzoate (2, 0.479 g, 1.89 mmol) was stirred in tetrahydrofuran (10.0 mL) at 0° C. Ethylbis(propan-2-yl)amine (0.825 mL, 4.72 mmol) was added was stirred at room temperature for 6 h. Acetic anhydride (0.841 g, 15.70 mmol) and N,N-dimethylpyridin-4-amine (0.192 g, 1.57 mmol) were added at 0° C., was stirred at room temperature for 12 h. After completion, the reaction mixture was concentrated, and the crude residue obtained was purified by silica gel column chromatography using ethyl acetate in hexane as eluents to afford the desired product (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(3-chlorobenzamido)propane-1,2-diyl diacetate (3). Yield: 0.70 g, 59% (over two steps); LCMS (ESI) m/z 750.71 [M+H]⁺.

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(3-chlorobenzamido)propane-1,2-diyl diacetate (5)

A mixture of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(3-chlorobenzamido)propane-1,2-diyl diacetate (3, 0.70 g, 0.93 mmol), 2-(2-(prop-2-yn-1-yloxy)ethoxy)ethan-1-ol (4, 0.403 g, 2.80 mmol) and activated powdered 4 Å molecular sieves (1.00 g, 100% w/w) in anhydrous dichloromethane (15.0 mL) was stirred at room temperature for 15 h. The solution was then cooled to −40° C. followed by addition of 1-iodopyrrolidine-2,5-dione (0.524 g, 2.33 mmol) and trifluoromethanesulfonic acid (0.140 mL, 0.93 mmol). The reaction mixture was stirred at −40° C. for another 2 h. After completion, the reaction mixture was quenched by addition of triethyl amine (neutral pH) and warmed up gradually to room temperature. The reaction mixture was filtered, and the filtrate was washed with deionized water. The organic layer was separated, dried, filtered, and concentrated. The crude residue obtained was purified by silica gel column chromatography using ethyl acetate in hexane as eluents to afford the desired product (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(3-chlorobenzamido)propane-1,2-diyl diacetate (5) as a mixture of anomers. Yield: 0.480 g, 66.8%; LCMS m/z 770.65 [M+H]+.

### Synthesis of (2R,4S,5R,6R)-6-((1R,2R)-3-(3-chlorobenzamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 201)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(3-chlorobenzamido)propane-1,2-diyl diacetate (5, 0.480 g, 0.62 mmol) in methanol (10 mL), was added a solution of lithium hydroxide monohydrate (0.090 g, 3.73 mmol) in water (0.4 mL). The reaction mixture was stirred at room temperature for 6 h. After completion, the reaction mixture was treated with acidic resin (Dowex 50, H⁺) up to pH ˜6 and the suspension was filtered. The filtrate was concentrated. The crude residue obtained was purified by prep HPLC using acetonitrile in water (+0.1% TFA) as eluents to afford the desired product (2R,4S,5R,6R)-6-((1R,2R)-3-(3-chlorobenzamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 201). Yield: 0.078 g, 21.3%; LCMS m/z 589.31 [M+H]+; ¹H NMR (400 MHz, methanol-d₄) δ 7.89-7.85 (m, 2H), 7.77-7.74 (m, 1H), 7.55-7.52 (m, 1H), 7.47 (t, J=7.6 Hz, 1H), 4.17 (d, J=2.4 Hz, 2H), 4.04-3.77 (m, 8H), 3.65-3.59 (m, 7H), 3.53-3.47 (m, 2H), 2.82 (t, J=2.4 Hz, 1H), 2.73 (dd, J=4.0 Hz & 12.8 Hz, 1H), 1.81-1.72 (m, 1H).

### (2R,4S,5R,6R)-6-((1R,2R)-3-(2-chlorobenzamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 27)

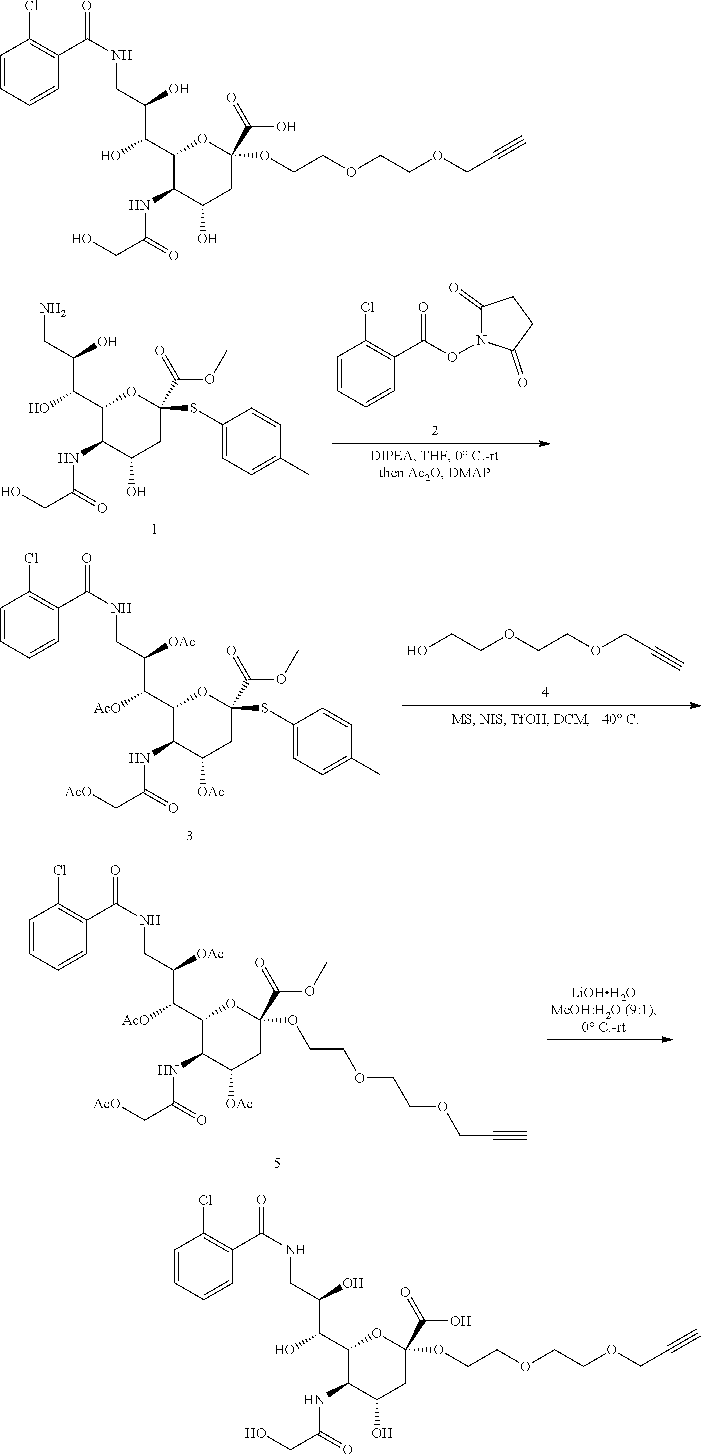

### Synthesis of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-chlorobenzamido)propane-1,2-diyl diacetate (3)

To a solution of methyl (2R,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (1, 0.6 g, 1.35 mmol) and 2,5-dioxopyrrolidin-1-yl 2-chlorobenzoate (2, 0.273 g, 1.08 mmol) in tetrahydrofuran (10 mL), was added ethylbis(propan-2-yl)amine (1.18 mL, 6.75 mmol) at 0° C. then stirred at room temperature for 12 h. After completion, acetic anhydride (1.02 mL, 10.8 mmol) and N,N-dimethylpyridin-4-amine (0.500 g, 4.09 mmol) were sequentially added to reaction mixture was further stirred at room temperature for 12 h. After completion, the reaction mixture was concentrated. The crude residue obtained was purified by silica gel column chromatography using methanol in dichloromethane as eluents to afford the desired product (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-chlorobenzamido)propane-1,2-diyl diacetate (3). Yield: 0.550 g, 54%; LCMS m/z 750.75 [M+H]⁺.

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-chlorobenzamido)propane-1,2-diyl diacetate (5)

A mixture of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-chlorobenzamido)propane-1,2-diyl diacetate (3, 0.550 g, 0.73 mmol), 2-(2-(prop-2-yn-1-yloxy)ethoxy)ethan-1-ol (4, 0.527 g, 3.66 mmol) and activated 4 Å powdered molecular sieves (0.5 g) in anhydrous dichloromethane (10 mL) was stirred at room temperature for 15 h then cooled down to −40° C. 1-Iodopyrrolidine-2,5-dione (0.412 g, 1.83 mmol) and trifluoromethanesulfonic acid (0.006 mL, 0.073 mmol) in dichloromethane (1.0 mL) were added at −40° C. and the resulting reaction mixture was stirred at this temperature for 1 h. After completion, the reaction mixture was quenched by addition of triethylamine (0.5 mL) and warmed up to room temperature. The reaction mixture was filtered, and the filtrate was washed with a saturated solution of sodium bicarbonate. The organic layer was dried, filtered, and concentrated. The crude residue obtained was purified by silica gel column chromatography using methanol in dichloromethane as eluents to afford the desired product (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-chlorobenzamido)propane-1,2-diyl diacetate (5). Yield: 0.300 g, 53%; LCMS m/z 787.90 [M+H3O]⁺.

### Synthesis of (2R,4S,5R,6R)-6-((1R,2R)-3-(2-chlorobenzamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 27)

To a solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-chlorobenzamido)propane-1,2-diyl diacetate (5, 0.300 g, 0.91 mmol) in methanol (10.0 mL) and water (1 mL), was added Lithium hydroxide monohydrate (0.223 g, 5.44 mmol) was stirred at room temperature for 6 h. After completion, the reaction mixture was treated with acidic resin (Dowex-50-H+) up to pH˜7 and the suspension was filtered. The filtrate was concentrated, and the crude residue obtained was purified by prep HPLC using acetonitrile in water (+0.1% TFA) as eluents to afford the desired product (2R,4S,5R,6R)-6-((1R,2R)-3-(2-chlorobenzamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 27). Yield: 10 mg, 5%; LCMS m/z 589.15 [M+H]+;¹H NMR (400 MHz, methanol-d₄) δ 7.50-7.36 (m, 4H), 4.18 (d, J=2.4 Hz, 2H), 4.03-3.99 (m, 3H), 3.95-3.84 (m, 3H), 3.81-3.76 (m, 2H), 3.66-3.60 (m, 7H), 3.55-3.49 (m, 2H), 2.83 (t, J=2.4 Hz, 1H), 2.74 (dd, J=12.0 Hz, 1H), 1.76 (t, J=12.0 Hz, 1H).

### (2R,4S,5R,6R)-6-((1R,2R)-1,2-dihydroxy-3-(4-hydroxybenzamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 198)

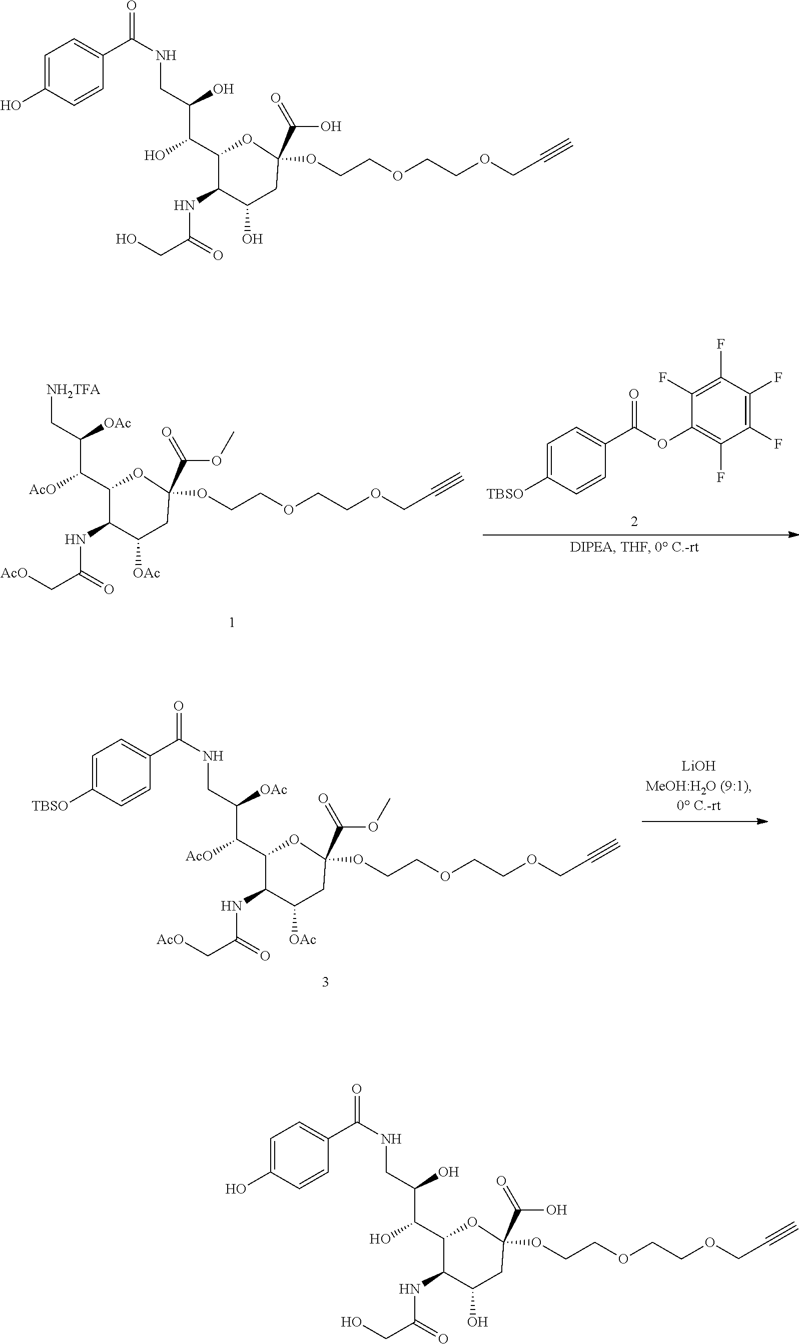

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(4-((tert-butyldimethylsilyl)oxy)benzamido)propane-1,2-diyl diacetate (3)

To a solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-((2,2,2-trifluoroacetyl)-14-azaneyl)propane-1,2-diyl diacetate (1, 0.20 g, 0.32 mmol) and perfluorophenyl 4-((tert-butyldimethylsilyl)oxy)benzoate (2, 0.159 g, 0.38 mmol) in tetrahydrofuran (6.0 mL) was added ethylbis(propan-2-yl)amine (0.28 mL, 1.58 mmol) at 0° C. then stirred at room temperature for 4 h. After completion the mixture was diluted with water and ethyl acetate. The organic layer was separated, dried, filtered, concentrated, and the crude residue obtained was purified by silica gel column chromatography using ethyl acetate in hexane as eluents to afford the desired product (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(4-((tert-butyldimethylsilyl)oxy)benzamido)propane-1,2-diyl diacetate (3). Yield: 0.90 g, 37%; LCMS (ESI) m/z 867.10 [M+H]⁺.

### Synthesis of (2R,4S,5R,6R)-6-((1R,2R)-1,2-dihydroxy-3-(4-hydroxybenzamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 198)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(4-((tert-butyldimethylsilyl)oxy)benzamido)propane-1,2-diyl diacetate (3, 0.09 g, 0.10 mmol) in methanol (4.0 mL), was added a solution of lithium hydroxide monohydrate (0.026 g, 0.60 mmol) in water (0.40 mL). The reaction mixture was stirred at room temperature for 6 h. After completion, the reaction mixture was treated with acidic resin (Dowex 50H⁺) up to pH ˜6 and the suspension was filtered. The filtrate was concentrated, and the crude residue obtained was purified by prep HPLC using acetonitrile in water (+0.1% TFA) as eluents to afford the desired product (2R,4S,5R,6R)-6-((1R,2R)-1,2-dihydroxy-3-(4-hydroxybenzamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 198). Yield: 0.0015 g, 2.5%; LCMS m/z 571.30 [M+H]+; ¹H NMR (400 MHz, methanol-d₄) δ 7.72 (d, J=8.0 Hz, 2H), 6.82 (d, J=8.0 Hz, 2H), 4.16 (d, J=2.4 Hz, 2H), 4.01-3.94 (m, 4H), 3.83-3.76 (m, 4H), 3.68-3.63 (m, 7H), 3.52-3.42 (m, 2H), 2.82-2.79 (m, 2H), 1.71 (m, 1H).

### (2R,4S,5R,6R)-6-((1R,2R)-1,2-dihydroxy-3-(3-hydroxybenzamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 29)

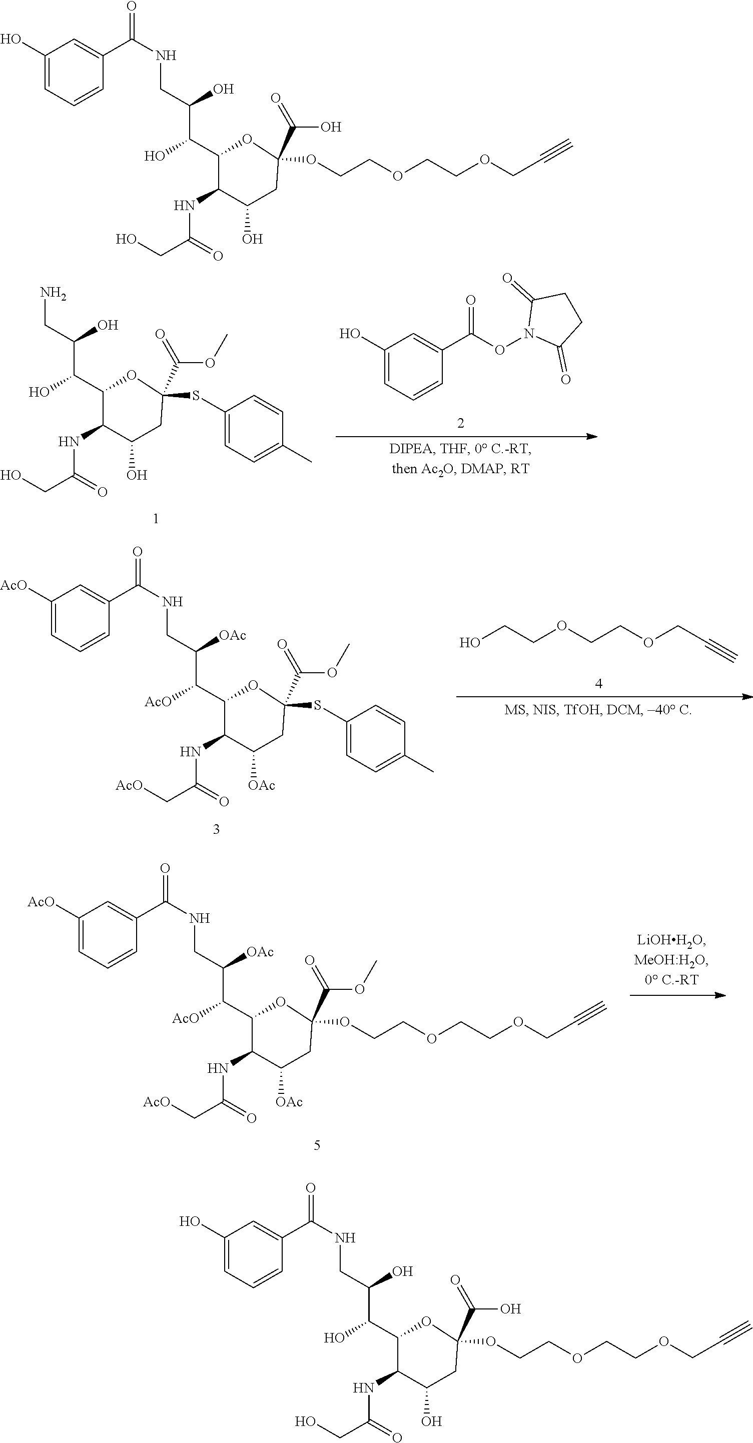

### Synthesis of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(3-acetoxybenzamido)propane-1,2-diyl diacetate (3)

To a solution of methyl (2R,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (1, 0.50 g, 1.28 mmol) and 2,5-dioxopyrrolidin-1-yl 3-hydroxybenzoate (2, 0.30 g, 1.28 mmol) in tetrahydrofuran (10.0 mL) at 0° C. was added ethylbis(propan-2-yl)amine (0.706 mL, 3.83 mmol) was stirred at room temperature for 6 h. Acetic anhydride (0.50 g, 12.8 mmol) and N,N-dimethylpyridin-4-amine (0.138 g, 1.28 mmol) were added at 0° C., was stirred at room temperature for 12 h. After completion, the reaction mixture was concentrated, and the crude residue obtained was purified by silica gel column chromatography using ethyl acetate in hexane as eluents to afford the desired product (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(3-acetoxybenzamido)propane-1,2-diyl diacetate (3). Yield: 0.331 g, 37.98% (over two steps); LCMS (ESI) m/z 774.90 [M+H]⁺.

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(3-acetoxybenzamido)propane-1,2-diyl diacetate (5)

A mixture of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(3-acetoxybenzamido)propane-1,2-diyl diacetate (3, 0.331 g, 0.427 mmol), 2-(2-(prop-2-yn-1-yloxy)ethoxy)ethan-1-ol (4, 0.290 g, 2.14 mmol) and activated powdered 4 Å molecular sieves (0.50 g, 100% w/w) in anhydrous dichloromethane (10.0 mL) was stirred at room temperature for 15 h. The solution was then cooled to −40° C. followed by addition of 1-iodopyrrolidine-2,5-dione (0.240 g, 1.07 mmol) and trifluoromethanesulfonic acid (0.037 mL, 0.427 mmol). The reaction mixture was stirred at the same temperature for another 2 h. After completion, the reaction mixture was quenched by addition of triethyl amine (neutral pH) and warmed up gradually to room temperature. The reaction mixture was filtered, and the filtrate was washed with deionized water. The organic layer was separated, dried, filtered, and concentrated. The crude residue obtained was purified by silica gel column chromatography using ethyl acetate in hexane as eluents to afford the desired product (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(3-acetoxybenzamido)propane-1,2-diyl diacetate (5) as a mixture of anomers. Yield: 0.250 g, 73.66%; LCMS m/z 792.60 [M−H]⁻.

### Synthesis of (2R,4S,5R,6R)-6-((1R,2R)-1,2-dihydroxy-3-(3-hydroxybenzamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 29)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(3-acetoxybenzamido)propane-1,2-diyl diacetate (5, 0.250 g, 0.31 mmol) in methanol (10 mL), was added a solution of lithium hydroxide monohydrate (0.079 g, 1.88 mmol) in water (0.4 mL). The reaction mixture was stirred at room temperature for 6 h. After completion, the reaction mixture was treated with acidic resin (Dowex 50, H⁺) up to pH ˜6 and the suspension was filtered. The filtrate was concentrated. The crude residue obtained was purified by prep HPLC using acetonitrile in water (+0.1% TFA) as eluents to afford the desired product (2R,4S,5R,6R)-6-((1R,2R)-1,2-dihydroxy-3-(3-hydroxybenzamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 29). Yield: 0.024 g, 13.4%; LCMS m/z 571.27 [M+H]+; ¹H NMR (400 MHz, methanol-d₄) δ 7.88-7.86 (m, 1H), 7.26-7.23 (m, 3H), 6.95-6.92 (m, 1H), 4.17 (d, J=2.4 Hz, 2H), 4.04-3.75 (m, 8H), 3.64-3.61 (m, 7H), 3.53-3.43 (m, 2H), 2.81 (t, J=2.4 Hz, 1H), 2.74 (dd, J=3.2 Hz & 12.4 Hz, 1H), 1.78-1.72 (m, 1H).

### (2R,4S,5R,6R)-6-((1R,2R)-1,2-dihydroxy-3-(2-hydroxybenzamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 28)

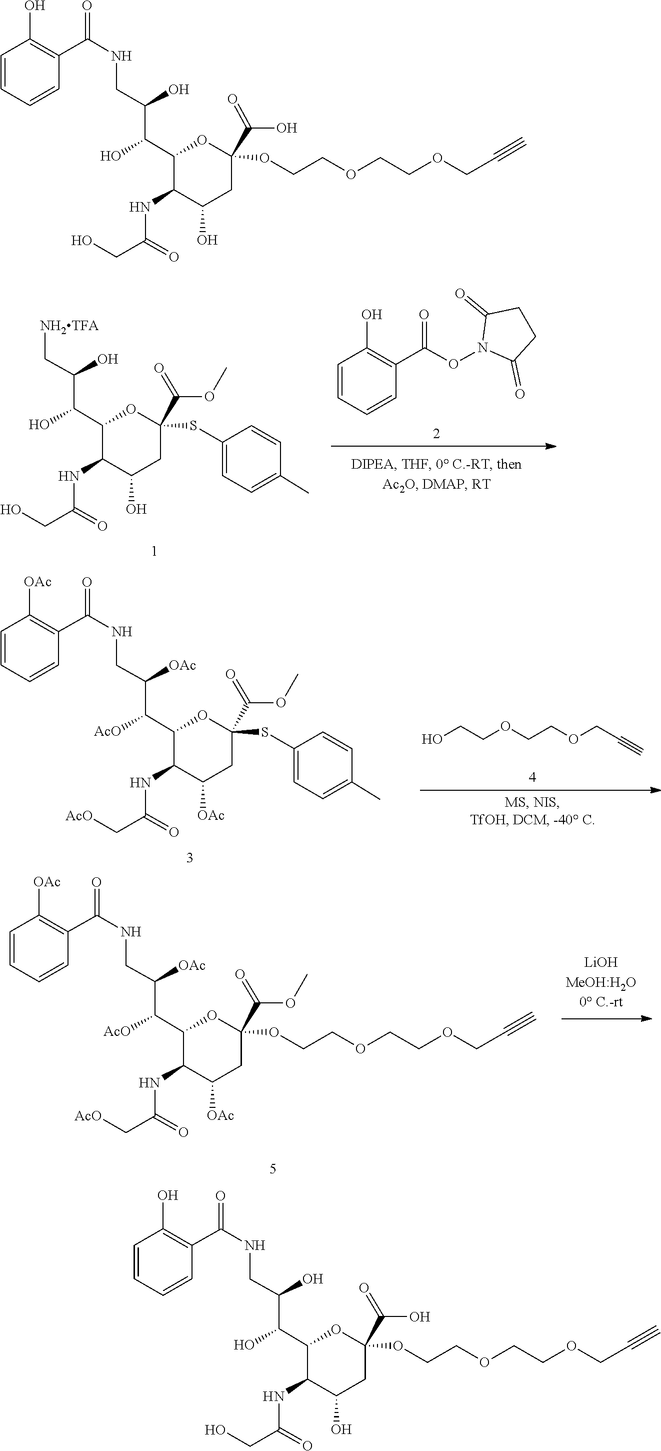

### Synthesis of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-acetoxybenzamido)propane-1,2-diyl diacetate (3)

To a stirred solution of methyl (2S,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (1, 0.80 g, 1.80 mmol) in tetrahydrofuran (10.0 mL) and N, N-dimethylformamide (2.00 mL), was added 2,5-dioxopyrrolidin-1-yl 2-hydroxybenzoate (2, 0.423 g, 1.80 mmol) and ethylbis(propan-2-yl)amine (1.57 mL, 9.0 mmol) at 0° C. then stirred at room temperature for 4 h. Acetic anhydride (1.5 mL, 18.0 mmol) and 4-dimethylaminopyridine (0.022 g, 0.18 mmol) was added at 0° C. and the resulting mixture was stirred at room temperature for 12 h. After completion the mixture was diluted with water and ethyl acetate. The organic layer was separated, dried, filtered, concentrated, and the crude residue obtained was purified by silica gel column chromatography using ethyl acetate in hexane as eluents to afford the desired product (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-acetoxybenzamido)propane-1,2-diyl diacetate (3). Yield: 0.430 g, 32.2%; LCMS (ESI) m/z 775.0 [M+H]⁺.

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-acetoxybenzamido)propane-1,2-diyl diacetate (5)

A mixture of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-acetoxybenzamido)propane-1,2-diyl diacetate (3, 0.430 g, 0.55 mmol), 2-(2-(prop-2-yn-1-yloxy)ethoxy)ethan-1-ol (4, 0.240 g, 1.66 mmol) and activated powdered 4 Å molecular sieves (0.50 g, 100% w/w) in anhydrous dichloromethane (10.0 mL) was stirred at room temperature for 15 h. The solution was then cooled to −40° C. followed by addition of 1-iodopyrrolidine-2,5-dione (0.330 g, 1.47 mmol) and trifluoromethanesulfonic acid (0.051 mL, 0.55 mmol). The reaction mixture was stirred at the same temperature for another 2 h. After completion, the reaction mixture was quenched by addition of triethyl amine (neutral pH) and warmed up gradually to room temperature. The reaction mixture was filtered, and the filtrate was washed with deionized water. The organic layer was separated, dried, filtered, and concentrated. The crude residue obtained was purified by silica gel column chromatography using ethyl acetate in hexane as eluents to afford the desired product (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-acetoxybenzamido)propane-1,2-diyl diacetate (5) as a mixture of anomers. Yield: 0.430 g, 93%; LCMS m/z 792.65 [M−H]−.

### Synthesis of (2R,4S,5R,6R)-6-((1R,2R)-1,2-dihydroxy-3-(2-hydroxybenzamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 28)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-acetoxybenzamido)propane-1,2-diyl diacetate (5, 0.430 g, 0.54 mmol) in methanol (5.0 mL), was added a solution of lithium hydroxide monohydrate (0.078 g, 3.25 mmol) in water (0.5 mL). The reaction mixture was stirred at room temperature for 4 h. After completion, the reaction mixture was treated with acidic resin (Dowex 50H⁺) up to pH 6 and the suspension was filtered. The filtrate was concentrated, and the crude residue obtained was purified by prep HPLC using acetonitrile in water (+0.1% TFA) as eluents to afford the desired product (2R,4S,5R,6R)-6-((1R,2R)-1,2-dihydroxy-3-(2-hydroxybenzamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 28). Yield: 0.060 g, 19.4%; LCMS m/z 571.25 [M+H]+; ¹H NMR (400 MHz, methanol-d₄) δ 7.90 (d, J=8.0 Hz, 1H), 7.81-7.86 (m, 1H), 7.38-7.34 (m, 1H), 6.91-6.87 (m, 1H), 4.16 (d, J=2.4 Hz, 2H), 4.03-3.75 (m, 8H), 3.66-3.58 (m, 7H), 3.51-3.44 (m, 2H), 2.81 (t, J=2.0 Hz, 1H), 2.73 (dd, J=3.6 Hz, 12.8 Hz, 1H), 1.80-1.74 (m, 1H).

### (2R,4S,5R,6R)-6-((1R,2R)-1,2-dihydroxy-3-(2-(4′-hydroxy-[1,1′-biphenyl]-4-yl)acetamido)propyl)-4-hydroxy-2-(2-(2-(prop-2-yn-1-yloxy)ethoxy)ethoxy)-5-(3-(2,2,2-trifluoroethyl)ureido)tetrahydro-2H-pyran-2-carboxylic acid (Compound 199)

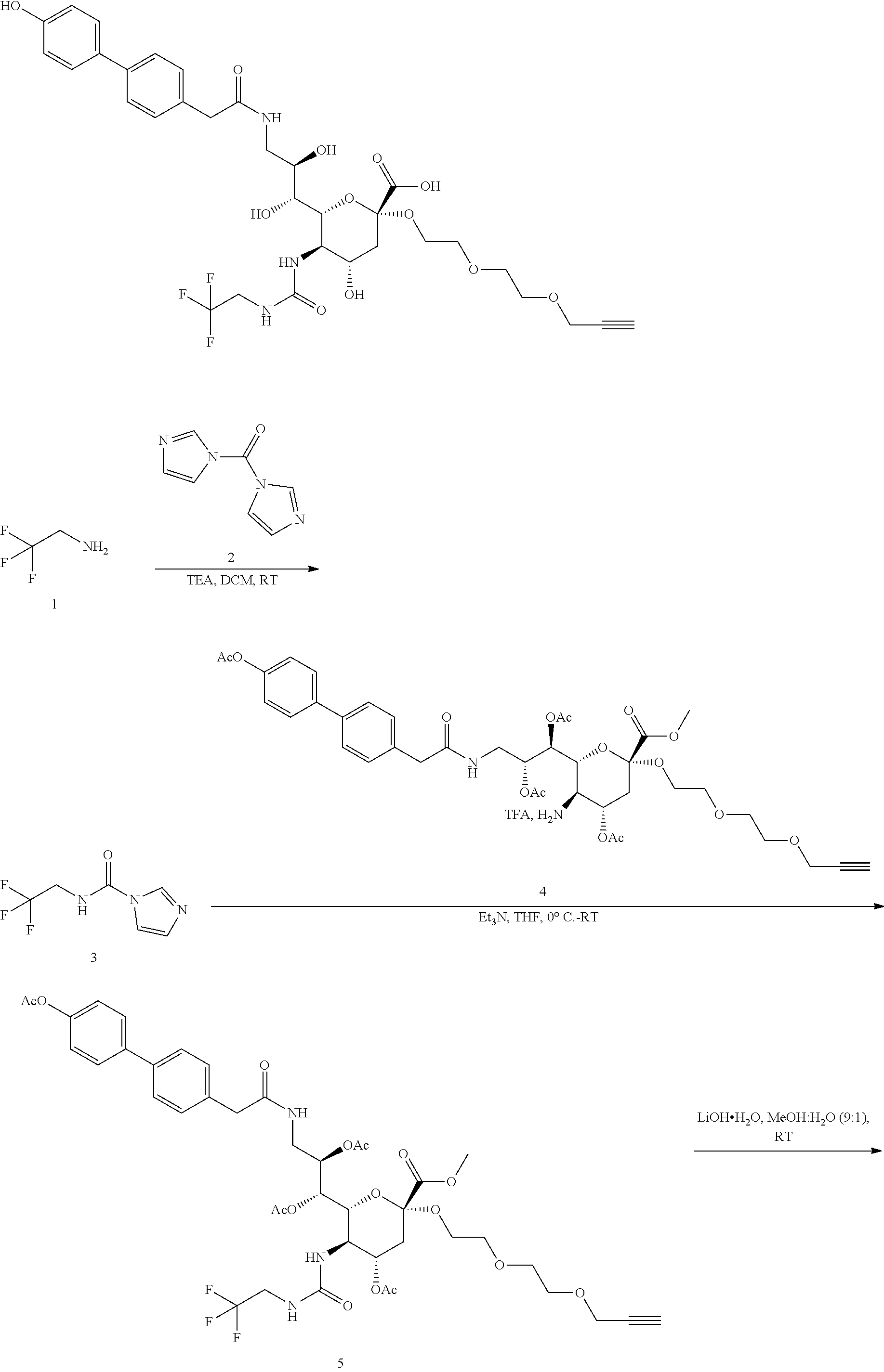

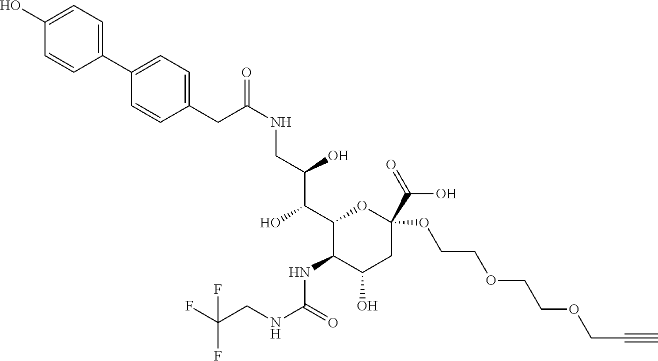 [SMILES: C#CCOCCOCCO[C@]1(C(=O)O)C[C@H](O)[C@@H](NC(=O)NCC(F)(F)F)[C@H]([C@H](O)[C@H](O)CNC(=O)Cc2ccc(-c3ccc(O)cc3)cc2)O1]

### Synthesis of N-(2,2,2-trifluoroethyl)-1H-imidazole-1-carboxamide (3)

To a stirred solution of 2,2,2-trifluoroethan-1-amine (1, 0.60 g, 6.06 mmol), in dichloromethane (6.0 mL), triethyl amine (4.135 mL, 30.30 mmol) and di(1H-imidazol-1-yl)methanone (2, 0.981, 6.06 mmol) were added at 0° C. and the mixture was stirred at room temperature for 1 h. After completion the mixture was concentrated to afford the desired product N-(2,2,2-trifluoroethyl)-1H-imidazole-1-carboxamide (3) as a crude. The crude was used to next step without purification. Yield: 0.60 g, 92%; LCMS m/z 194.15 [M+H3O]⁺.

### Synthesis of (1R,2R)-1-((2R,3R,4S,6R)-4-acetoxy-6-(methoxycarbonyl)-6-(2-(2-(prop-2-yn-1-yloxy)ethoxy)ethoxy)-3-(3-(2,2,2-trifluoroethyl)ureido)tetrahydro-2H-pyran-2-yl)-3-(2-(4′-acetoxy-[1,1′-biphenyl]-4-yl)acetamido)propane-1,2-diyl diacetate (5)

To a stirred solution of (1R,2R)-1-((2R,3R,4S,6R)-4-acetoxy-6-(methoxycarbonyl)-6-(2-(2-(prop-2-yn-1-yloxy)ethoxy)ethoxy)-3-((2,2,2-trifluoroacetyl)-14-azaneyl)tetrahydro-2H-pyran-2-yl)-3-(2-(4′-acetoxy-[1,1′-biphenyl]-4-yl)acetamido)propane-1,2-diyl diacetate (4, 0.120 g, 0.14 mmol) and N-(2,2,2-trifluoroethyl)-1H-imidazole-1-carboxamide (3, 0.078 g, 0.41 mmol) in tetrahydrofuran (3.0 mL) was added triethyl amine (0.09 mL, 0.680 mmol) at 0° C. then stirred at room temperature for 12 h. After completion the mixture was diluted with water and ethyl acetate. The organic layer was separated, dried, filtered, concentrated, and the crude residue obtained was purified by silica gel column chromatography using ethyl acetate in hexane as eluents to afford the desired product (1R,2R)-1-((2R,3R,4S,6R)-4-acetoxy-6-(methoxycarbonyl)-6-(2-(2-(prop-2-yn-1-yloxy)ethoxy)ethoxy)-3-(3-(2,2,2-trifluoroethyl)ureido)tetrahydro-2H-pyran-2-yl)-3-(2-(4′-acetoxy-[1,1′-biphenyl]-4-yl)acetamido)propane-1,2-diyl diacetate (5). Yield: 0.120 g, 96%; LCMS (ESI) m/z 910.20 [M+H]⁺.

### Synthesis of (2R,4S,5R,6R)-6-((1R,2R)-1,2-dihydroxy-3-(2-(4′-hydroxy-[1,1′-biphenyl]-4-yl)acetamido)propyl)-4-hydroxy-2-(2-(2-(prop-2-yn-1-yloxy)ethoxy)ethoxy)-5-(3-(2,2,2-trifluoroethyl)ureido)tetrahydro-2H-pyran-2-carboxylic acid (Cpd. 199)

To a stirred solution of (1R,2R)-1-((2R,3R,4S,6R)-4-acetoxy-6-(methoxycarbonyl)-6-(2-(2-(prop-2-yn-1-yloxy)ethoxy)ethoxy)-3-(3-(2,2,2-trifluoroethyl)ureido)tetrahydro-2H-pyran-2-yl)-3-(2-(4′-acetoxy-[1,1′-biphenyl]-4-yl)acetamido)propane-1,2-diyl diacetate (5, 0.120 g, 0.13 mmol) in methanol (3.0 mL), was added a solution of lithium hydroxide monohydrate (0.034 g, 0.79 mmol) in water (0.30 mL). The reaction mixture was stirred at room temperature for 6 h. After completion, the reaction mixture was treated with acidic resin (Dowex 50H⁺) up to pH ˜6 and the suspension was filtered. The filtrate was concentrated, and the crude residue obtained was purified by prep HPLC using acetonitrile in water (+0.1% TFA) as eluents to afford the desired product (2R,4S,5R,6R)-6-((1R,2R)-1,2-dihydroxy-3-(2-(4′-hydroxy-[1,1′-biphenyl]-4-yl)acetamido)propyl)-4-hydroxy-2-(2-(2-(prop-2-yn-1-yloxy)ethoxy)ethoxy)-5-(3-(2,2,2-trifluoroethyl)ureido)tetrahydro-2H-pyran-2-carboxylic acid (Cpd. 199). Yield: 0.012 g, 13%; LCMS m/z 728.35 [M+H]+; ¹H NMR (400 MHz, methanol-d₄) δ 7.49 (d, J=8.4 Hz, 2H), 7.45 (d, J=6.8 Hz, 2H), 7.32 (d, J=8.4 Hz, 2H), 6.84 (d, J=6.8 Hz, 2H), 4.16 (d, J=2.4 Hz, 2H), 3.90-3.78 (m, 4H), 3.65-3.42 (m, 13H), 3.43 (d, J=8.8 Hz, 1H), 3.26-3.23 (m, 1H), 2.82 (t, J=2.4 Hz, 1H), 2.71 (dd, J=12.4 & 4.0 Hz, 1H), 1.74 (t, J=12.4 Hz, 1H).

### (2R,4S,5R,6R)-6-((1R,2R)-1,2-dihydroxy-3-(2-((1s,4S)-4-hydroxycyclohexyl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 200)

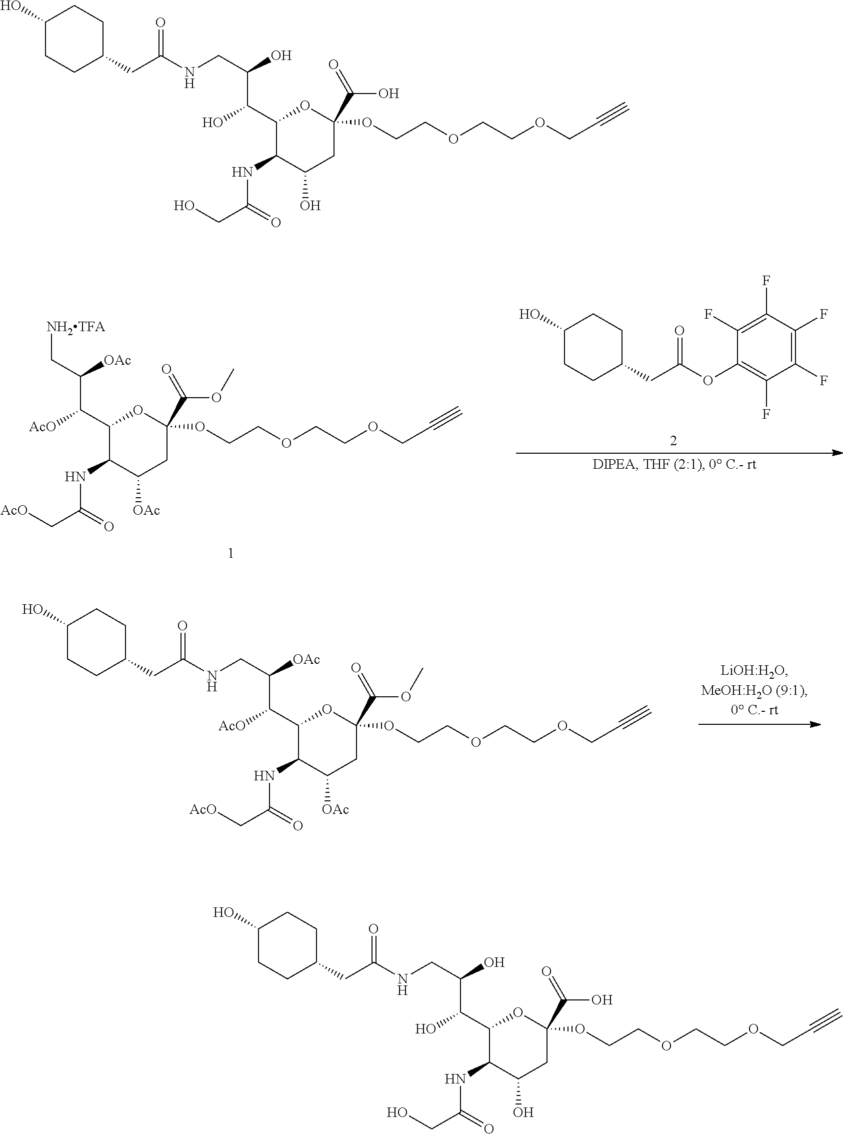

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-((1s,4S)-4-hydroxycyclohexyl)acetamido)propane-1,2-diyl diacetate (3)

To a solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-aminopropane-1,2-diyl diacetate (1, 0.400 g, 0.63 mmol), in tetrahydrofuran (5.0 mL), were successively added ethylbis(propan-2-yl)amine (0.550 mL, 3.16 mmol) and perfluorophenyl 2-((1s,4s)-4-hydroxycyclohexyl)acetate (0.164 g, 0.51 mmol) at 0° C. then stirred at room temperature for 6 h. After completion the mixture was concentrated. that ended up with crude residue. The crude residue obtained was purified by silica gel column chromatography using ethyl acetate in hexane as eluents to afford the desired product (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-((1s,4S)-4-hydroxycyclohexyl)acetamido)propane-1,2-diyl diacetate (3). Yield: 0.250 g, 69%; ELSD m/z 773.35 [M+H]⁺.

### Synthesis of (2R,4S,5R,6R)-6-((1R,2R)-1,2-dihydroxy-3-(2-((1s,4S)-4-hydroxycyclohexyl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 200)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-((1s,4S)-4-hydroxycyclohexyl)acetamido)propane-1,2-diyl diacetate (3, 0.250 g, 0.32 mmol) in methanol (5 mL), was added a solution of lithium hydroxide monohydrate (0.080 g, 1.94 mmol) in water (0.5 mL). The reaction mixture was stirred at room temperature for 6 h. After completion, the reaction mixture was treated with acidic resin (Dowex 50, H⁺) up to Ph ˜6 and the suspension was filtered. The filtrate was concentrated. The crude residue obtained was purified by prep HPLC using acetonitrile in water (+0.1% TFA) as eluents to afford the desired product (2R,4S,5R,6R)-6-((1R,2R)-1,2-dihydroxy-3-(2-((1s,4S)-4-hydroxycyclohexyl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 200). Yield: 33.0 mg, 17%; ELSD m/z 591.25 [M+H]⁺; ¹H NMR (400 MHz, methanol-d₄) δ 4.19 (d, J=2.4 Hz, 2H), 4.03 (brs, 2H), 3.94-3.79 (m, 5H), 3.75-3.72 (m, 1H), 3.69-3.58 (m, 8H), 3.37 (dd, J=8.8 & 1.2 Hz, 1H), 3.31-3.22 (m, 1H), 2.84 (t, J=2.4 Hz, 1H), 2.73 (dd, J=12.8 & 4.8 Hz, 1H), 2.07 (d, J=7.6 Hz, 2H), 1.88-1.68 (m, 4H), 1.59-1.42 (m, 6H).

### (2R,4S,5R,6R)-6-((1R,2R)-1,2-dihydroxy-3-(2-(2-hydroxy-[1,1′-biphenyl]-4-yl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 66)

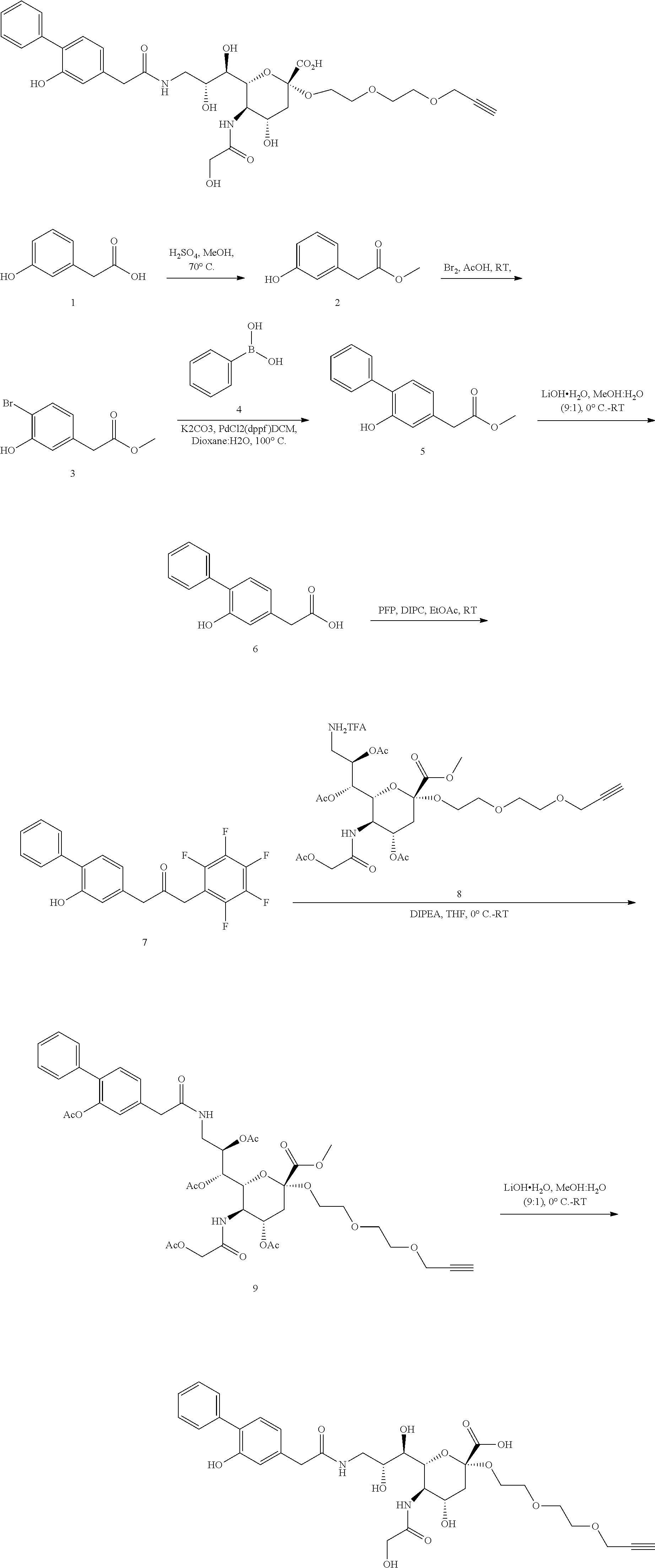

### Synthesis of methyl 2-(3-hydroxyphenyl)acetate (2)

To a stirred solution of 2-(3-hydroxyphenyl)acetic acid (1, 4.00 g, 26.30 mmol), in methanol (40.0 mL), was added sulfuric acid (8.0 mL) at 0° C. and the mixture was stirred at 60° C. for 5 h. After completion the mixture was diluted with water and ethyl acetate. The organic layer was separated, dried, filtered, concentrated, and the crude residue obtained was purified by silica gel column chromatography using ethyl acetate in hexane as eluents to afford the desired product methyl 2-(3-hydroxyphenyl)acetate (2). Yield: 4.0 g, 92%; LCMS m/z 165.10 [M−HO]⁻.

### Synthesis of methyl 2-(4-bromo-3-hydroxyphenyl)acetate (3)

To a stirred solution of methyl 2-(3-hydroxyphenyl)acetate (2, 1.50 g, 9.03 mmol) in acetic acid (75.0 mL), was added bromine (0.46 mL, 9.03 mmol) at 15° C. and the mixture was stirred at room temperature for 1 h. After completion, the reaction mixture was diluted with water and ethyl acetate. The organic layer was separated, dried, filtered, concentrated, and the crude residue obtained was purified by silica gel column chromatography using ethyl acetate in hexane as eluents to afford the desired product methyl 2-(4-bromo-3-hydroxyphenyl)acetate (3). Yield: 1.70 g, 77%; LCMS m/z 244.90 [M−H]−.

### Synthesis of methyl 2-(2-hydroxy-[1,1′-biphenyl]-4-yl)acetate (5)

A mixture of methyl 2-(4-bromo-3-hydroxyphenyl)acetate (3, 1.70 g, 6.94 mmol), phenylboronic acid (4, 0.846 g, 6.94 mmol) and potassium carbonate (2.88 g, 20.80 mmol) in 1,4-dioxane (18.0 mL) and water (2.0 mL) was degassed by bubbling argon for 10 min. 1,1-bis(Diphenylphosphino)ferrocene dichloropalladium (0.566 g, 0.69 mmol) was added at room temperature and the resulting mixture was stirred for 8 h at 100° C. After completion the mixture was diluted with water and ethyl acetate. The organic layer was separated, dried, filtered, concentrated, and the crude residue obtained was purified by silica gel column chromatography using ethyl acetate in hexane as eluents to afford the desired product methyl 2-(2-hydroxy-[1,1′-biphenyl]-4-yl)acetate (5). Yield: 1.50 g, 89%; LCMS m/z 241.05 [M+H3O]⁺.

### Synthesis of 2-(2-hydroxy-[1,1′-biphenyl]-4-yl)acetic acid (6)

To a stirred solution of methyl 2-(2-hydroxy-[1,1′-biphenyl]-4-yl)acetate (5, 1.50 g, 6.19 mmol) in methanol (15.0 mL), was added a solution of lithium hydroxide monohydrate (0.780 g, 18.59 mmol) in water (1.50 mL). The reaction mixture was stirred at room temperature for 6 h. After completion, the reaction mixture was treated with 1N HCl and the mixture was diluted with water and ethyl acetate. The organic layer was separated, dried, filtered, and concentrated to afford the desired product 2-(2-hydroxy-[1,1′-biphenyl]-4-yl)acetic acid (6) as a crude. Yield: 1.20 g, 85%; LCMS m/z 227.05 [M−H]⁻.

### Synthesis of perfluorophenyl 2-(2-hydroxy-[1,1′-biphenyl]-4-yl)acetate (7)

To a stirred solution of 2-(2-hydroxy-[1,1′-biphenyl]-4-yl)acetic acid (6, 1.20 g, 5.26 mmol) in ethyl acetate (12.0 mL), were added pentafluorophenol (0.967 g, 5.25 mmol) and N,N′-diisopropylcarbodiimide (0.82 mL, 5.26 mmol) at 0° C. and the reaction was stirred at room temperature for 3 h. After completion the mixture was filtered, and washed with ethyl acetate, the filtrate was concentrated, and the crude residue obtained was purified by silica gel column chromatography using ethyl acetate in hexane as eluents to afford the desired product perfluorophenyl 2-(2-hydroxy-[1,1′-biphenyl]-4-yl)acetate (7). Yield: 1.20 g, 58%; ¹H NMR (400 MHz, DMSO-d₆) δ 9.60 (s, 1H), 7.43-4.33 (m, 3H), 7.24 (d, J=6.8 Hz, 2H), 7.08 (d, J=8.4 Hz, 1H), 6.88 (d, J=2.8 Hz, 1H), 6.79 (dd, J=8.4 & 2.8 Hz, 1H), 4.01 (s, 2H).

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(2-acetoxy-[1,1′-biphenyl]-4-yl)acetamido)propane-1,2-diyl diacetate (9)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-aminopropane-1,2-diyl diacetate (8, 0.50 g, 0.69 mmol) was added perfluorophenyl 2-(2-hydroxy-[1,1′-biphenyl]-4-yl)acetate (7, 0.27 g, 0.69 mmol) in tetrahydrofuran (5.0 mL) followed by ethylbis(propan-2-yl)amine (0.35 mL, 2.05 mmol) at 0° C. then stirred at room temperature for 12 h. After completion the mixture was diluted with water and ethyl acetate. The organic layer was separated, dried, filtered, concentrated, and the crude residue obtained was purified by silica gel column chromatography using ethyl acetate in hexane as eluents to afford the desired product (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(2-acetoxy-[1,1′-biphenyl]-4-yl)acetamido)propane-1,2-diyl diacetate (9). Yield: 0.250 g, 41%; LCMS (ESI) m/z 843.20 [M+H]⁺.

### Synthesis of (2R,4S,5R,6R)-6-((1R,2R)-1,2-dihydroxy-3-(2-(2-hydroxy-[1,1′-biphenyl]-4-yl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 66)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(2-acetoxy-[1,1′-biphenyl]-4-yl)acetamido)propane-1,2-diyl diacetate (9, 0.250 g, 0.28 mmol) in methanol (3.0 mL), was added a solution of lithium hydroxide monohydrate (0.072 g, 1.69 mmol) in water (0.30 mL). The reaction mixture was stirred at room temperature for 6 h. After completion, the reaction mixture was treated with acidic resin (Dowex 50H+) up to pH ˜6 and the suspension was filtered. The filtrate was concentrated, and the crude residue obtained was purified by prep HPLC using acetonitrile in water (+0.1% TFA) as eluents to afford the desired product (2R,4S,5R,6R)-6-((1R,2R)-1,2-dihydroxy-3-(2-(2-hydroxy-[1,1′-biphenyl]-4-yl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 66). Yield: 0.011 g, 6%; LCMS m/z 661.40[M+H]⁺; ¹H NMR (400 MHz, methanol-d₄) δ 7.39-36 (m, 2H), 7.31-7.27 (m, 3H), 7.06 (d, J=8.4 Hz, 1H), 6.79 (d, J=2.4 Hz, 1H), 6.75 (dd, J=8.4 & 2.8 Hz, 1H), 4.17 (d, J=2.4 Hz, 2H), 4.01 (s, 2H), 3.93-3.90 (m, 1H), 3.86-3.76 (m, 3H), 3.71-3.66 (m, 1H), 3.65-3.59 (m, 8H), 3.45 (s, 2H), 3.35-3.34 (m, 1H), 3.18-3.12 (m, 1H), 2.81 (t, J=2.4 Hz, 1H), 2.75 (dd, J=12.4 & 4.0 Hz, 1H), 1.71 (t, J=12.4 Hz, 1H).

### (2R,4S,5R,6R)-6-((1R,2R)-1,2-dihydroxy-3-(2-(3-hydroxy-[1,1′-biphenyl]-4-yl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 67)

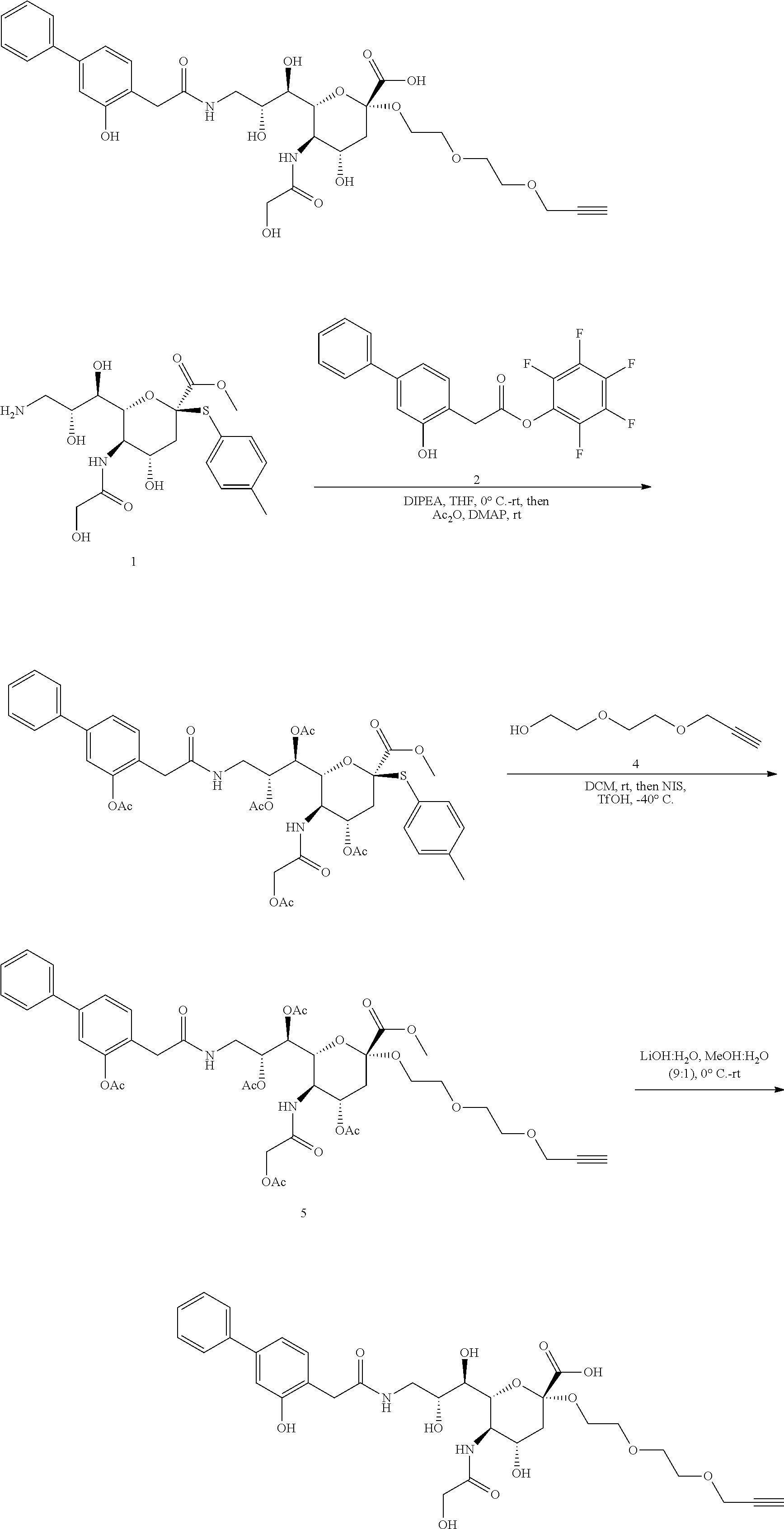

### Synthesis of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(3-acetoxy-[1,1′-biphenyl]-4-yl)acetamido)propane-1,2-diyl diacetate (3)

To a solution of methyl (2R,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (1, 0.400 g, 0.89 mmol), in tetrahydrofuran (10.0 mL), were added ethylbis(propan-2-yl)amine (0.70 mL, 4.50 mmol) and perfluorophenyl 2-(3-hydroxy-[1,1′-biphenyl]-4-yl)acetate (2, 0.532 g, 1.35 mmol) at 0° C. then stirred at room temperature for 6 h. Acetic anhydride (1.60 mL, 17.80 mmol) and 4-dimethylaminopyridine (0.109 g, 0.900 mmol) were added at 0° C. and the resulting mixture was stirred at room temperature for 12 h. After completion the mixture was diluted with water and ethyl acetate. The organic layer was separated, dried, filtered, concentrated, and the crude residue obtained was purified by silica gel column chromatography using ethyl acetate in hexane as eluents to afford the desired product (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(3-acetoxy-[1,1′-biphenyl]-4-yl)acetamido)propane-1,2-diyl diacetate (3). Yield: 0.250 g, 32%; LCMS (ESI) m/z 865.95 [M+H]⁺.

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(3-acetoxy-[1,1′-biphenyl]-4-yl)acetamido)propane-1,2-diyl diacetate (5)

A mixture of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(3-acetoxy-[1,1′-biphenyl]-4-yl)acetamido)propane-1,2-diyl diacetate (3, 0.550 g, 0.64 mmol), 2-(2-(prop-2-yn-1-yloxy)ethoxy)ethan-1-ol (4, 0.275 g, 1.91 mmol) and activated powdered 4 Å molecular sieves (1.00 g, 100% w/w) in anhydrous dichloromethane (10.0 mL) was stirred at room temperature for 15 h. The solution was then cooled to −40° C. followed by addition of 1-iodopyrrolidine-2,5-dione (0.275 g, 0.95 mmol) and trifluoromethanesulfonic acid (0.08 mL, 0.924 mmol). The reaction mixture was stirred at the same temperature for another 1 h. After completion, the reaction mixture was quenched by addition of triethyl amine (neutral pH) and warmed up gradually to room temperature. The reaction mixture was filtered, and the filtrate was washed with deionized water. The organic layer was separated, dried, filtered, and concentrated. The crude residue obtained was purified by silica gel column chromatography using ethyl acetate in hexane as eluents to afford the desired product (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(3-acetoxy-[1,1′-biphenyl]-4-yl)acetamido)propane-1,2-diyl diacetate (5) as a mixture of anomers. Yield: 0.400 g, 71%; LCMS m/z 885.35 [M+H]⁺.

### Synthesis of (2R,4S,5R,6R)-6-((1R,2R)-1,2-dihydroxy-3-(2-(3-hydroxy-[1,1′-biphenyl]-4-yl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 67)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(3-acetoxy-[1,1′-biphenyl]-4-yl)acetamido)propane-1,2-diyl diacetate (5, 0.750 g, 0.84 mmol) in methanol (10.0 mL), was added a solution of lithium hydroxide monohydrate (0.121 g, 5.04 mmol) in water (0.5 mL). The reaction mixture was stirred at room temperature for 6 h. After completion, the reaction mixture was treated with acidic resin (Dowex 50H⁺) up to pH ˜6 and the suspension was filtered. The filtrate was concentrated, and the crude residue obtained was purified by prep HPLC using acetonitrile in water (+0.1% TFA) as eluents to afford the desired product (2R,4S,5R,6R)-6-((1R,2R)-1,2-dihydroxy-3-(2-(3-hydroxy-[1,1′-biphenyl]-4-yl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 67). Yield: 0.018 g, 3.21%; LCMS m/z 661.45 [M+H]⁺; ¹H NMR (400 MHz, methanol-d₄) δ 7.58-7.56 (m, 2H), 7.42-7.38 (m, 2H), 7.33-7.29 (m, 1H), 7.22-7.20 (m, 1H), 7.08-7.06 (m, 2H), 4.17 (d, J=2.4 Hz, 2H), 4.02 (brs, 2H), 3.91-3.78 (m, 4H), 3.73-3.71 (m, 1H), 3.68-3.58 (m, 10H), 3.38 (dd, J=8.8 & 1.6 Hz, 1H), 3.31-3.27 (m, 1H), 2.82 (t, J=2.4 Hz, 1H), 2.72 (dd, J=12.0 & 3.2 Hz, 1H), 1.77-1.71 (m, 1H).

### (2R,4S,5R,6R)-6-((1R,2R)-1,2-dihydroxy-3-(2-(2′-hydroxy-[1,1′-biphenyl]-4-yl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 68)

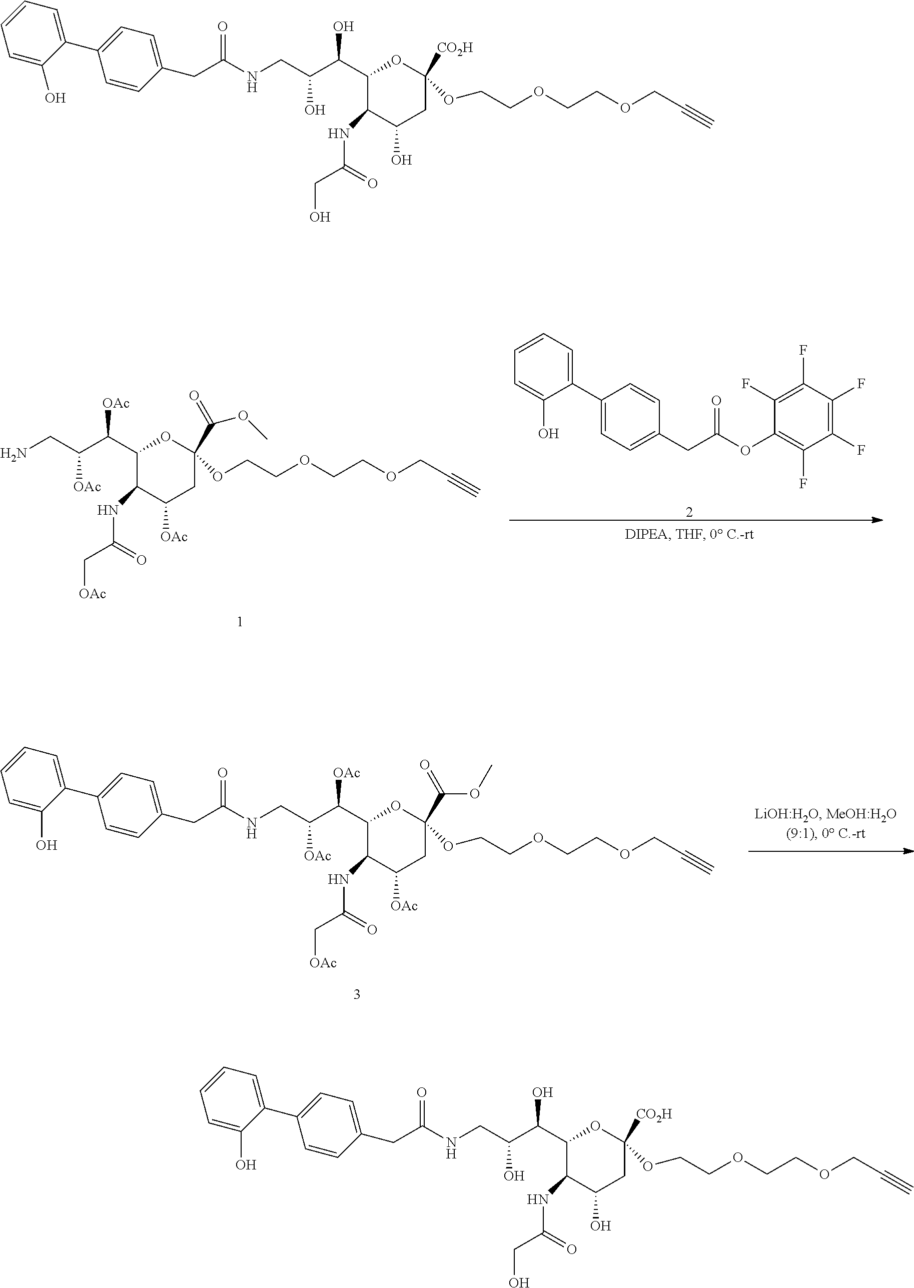

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(2′-hydroxy-[1,1′-biphenyl]-4-yl)acetamido)propane-1,2-diyl diacetate (3)

To a solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-aminopropane-1,2-diyl diacetate (1, 0.400 g, 0.59 mmol), in tetrahydrofuran (5.0 mL), were added ethylbis(propan-2-yl)amine (0.50 mL, 2.94 mmol) and perfluorophenyl 2-(2′-hydroxy-[1,1′-biphenyl]-4-yl)acetate (2, 0.348 g, 0.88 mmol) at 0° C. then stirred at room temperature for 6 h. Acetic anhydride (1.60 mL, 17.80 mmol) and 4-dimethylaminopyridine (0.109 g, 0.900 mmol) were added at 0° C. and the resulting mixture was stirred at room temperature for 12 h. After completion the mixture was diluted with water and ethyl acetate. The organic layer was separated, dried, filtered, concentrated, and the crude residue obtained was purified by silica gel column chromatography using ethyl acetate in hexane as eluents to afford the desired product (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(2′-hydroxy-[1,1′-biphenyl]-4-yl)acetamido)propane-1,2-diyl diacetate (3). Yield: 0.250 g, 54%; LCMS (ESI) m/z 840.90 [M−H]⁻.

### Synthesis of (2R,4S,5R,6R)-6-((1R,2R)-1,2-dihydroxy-3-(2-(2′-hydroxy-[1,1′-biphenyl]-4-yl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 68)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(2′-hydroxy-[1,1′-biphenyl]-4-yl)acetamido)propane-1,2-diyl diacetate (3, 0.150 g, 0.17 mmol) in methanol (5.0 mL), was added a solution of lithium hydroxide monohydrate (0.024 g, 1.02 mmol) in water (0.5 mL). The reaction mixture was stirred at room temperature for 6 h. After completion, the reaction mixture was treated with acidic resin (Dowex 50H⁺) up to pH ˜6 and the suspension was filtered. The filtrate was concentrated, and the crude residue obtained was purified by prep HPLC using acetonitrile in water (+0.1% TFA) as eluents to afford the desired product (2R,4S,5R,6R)-6-((1R,2R)-1,2-dihydroxy-3-(2-(2′-hydroxy-[1,1′-biphenyl]-4-yl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 68). Yield: 0.015 g 13%; LCMS m/z 661.35 [M+H]⁺; ¹H NMR (400 MHz, methanol-d₄) δ 7.50 (d, J=8.0 Hz, 2H), 7.33 (d, J=8.4 Hz, 2H), 7.23 (d, J=7.2 Hz, 1H), 7.15-7.11 (m, 1H), 6.89-6.86 (m, 2H), 4.17 (d, J=2.4 Hz, 2H), 4.03 (brs, 2H), 3.98-3.78 (m, 4H), 3.74-3.71 (m, 1H), 3.69-3.58 (m, 10H), 3.37 (d, J=8.8 Hz, 1H), 3.29-3.22 (m, 1H), 2.82 (t, J=2.0 Hz, 1H), 2.73 (dd, J=11.6 & 3.6 Hz, 1H), 1.77-1.71 (m, 1H).

### (2R,4S,5R,6R)-6-((1R,2R)-3-(2-([1,1′-biphenyl]-3-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 33)

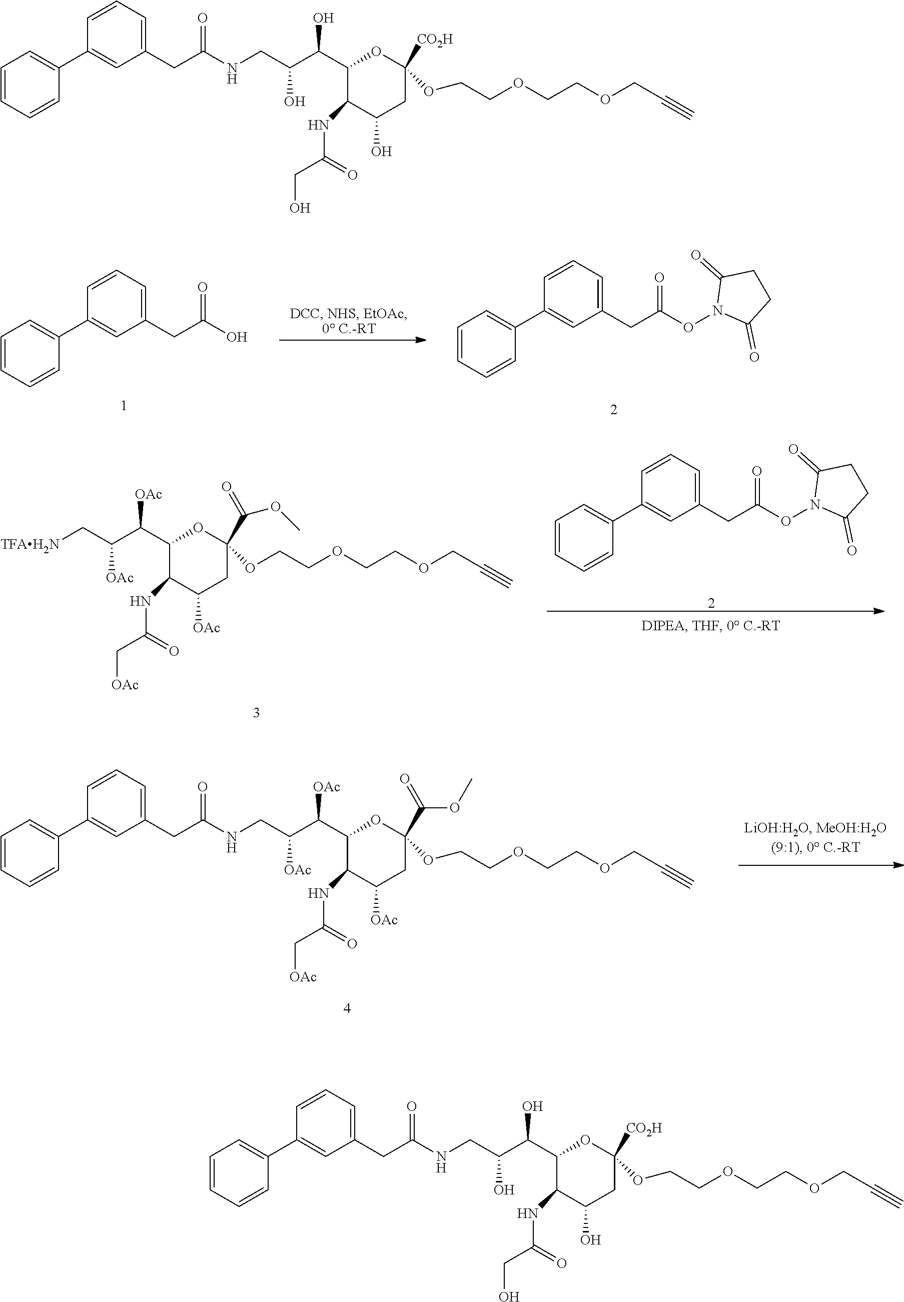

### Synthesis of 2,5-dioxopyrrolidin-1-yl 2-([1,1′-biphenyl]-3-yl)acetate (2)

To a stirred solution of 2-([1,1′-biphenyl]-3-yl)acetic acid (1, 0.50 g, 2.36 mmol) in ethyl acetate (10.0 mL), 1-hydroxypyrrolidine-2,5-dione (0.271 g, 2.36 mmol) and N,N′-dicyclohexylcarbodiimide (0.559 g, 2.36 mmol) was added at 0° C. and the reaction was stirred at room temperature for 12 h. After completion the mixture was filtered, and washed with ethyl acetate, the filtrate was concentrated, and the crude residue obtained was purified by silica gel column chromatography using ethyl acetate in hexane as eluents to afford the desired product 2,5-dioxopyrrolidin-1-yl 2-([1,1′-biphenyl]-3-yl)acetate (2). Yield: 0.40 g, 55%; ¹H NMR (400 MHz, DMSO-d₆) δ 7.67-7.66 (m, 3H), 7.61 (d, J=8.0 Hz, 1H), 7.49-7.44 (m, 3H), 7.37 (q, J=7.6 Hz, 2H), 4.19 (s, 2H), 2.81 (s, 4H).

### Synthesis of (1R,2R)-3-(2-([1,1′-biphenyl]-3-yl)acetamido)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)propane-1,2-diyl diacetate (4)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-aminopropane-1,2-diyl diacetate (3, 0.40 g, 0.55 mmol) and 2,5-dioxopyrrolidin-1-yl 2-([1,1′-biphenyl]-3-yl)acetate (2, 0.169 g, 0.55 mmol) in tetrahydrofuran (4.0 mL) was added ethylbis(propan-2-yl)amine (0.477 mL, 2.74 mmol) at 0° C. then stirred at room temperature for 12 h. After completion the mixture was diluted with water and ethyl acetate. The organic layer was separated, dried, filtered, concentrated, and the crude residue obtained was purified by silica gel column chromatography using ethyl acetate in hexane as eluents to afford the desired product (1R,2R)-3-(2-([1,1′-biphenyl]-3-yl)acetamido)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)propane-1,2-diyl diacetate (4). Yield: 0.300 g, 66%; LCMS (ESI) m/z 826.90 [M+H]⁺.

### Synthesis of (2R,4S,5R,6R)-6-((1R,2R)-3-(2-([1,1′-biphenyl]-3-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 33)

To a stirred solution of (1R,2R)-3-(2-([1,1′-biphenyl]-3-yl)acetamido)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)propane-1,2-diyl diacetate (4, 0.300 g, 0.36 mmol) in methanol (3.0 mL), was added a solution of lithium hydroxide monohydrate (0.091 g, 2.18 mmol) in water (0.30 mL). The reaction mixture was stirred at room temperature for 6 h. After completion, the reaction mixture was treated with acidic resin (Dowex 50H⁺) up to pH ˜6 and the suspension was filtered. The filtrate was concentrated, and the crude residue obtained was purified by prep HPLC using acetonitrile in water (+0.1% TFA) as eluents to afford the desired product (2R,4S,5R,6R)-6-((1R,2R)-3-(2-([1,1′-biphenyl]-3-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 33). Yield: 0.015 g, 6%; LCMS m/z 645.48 [M+H]+; ¹H NMR (400 MHz, methanol-d₄) δ 7.63-7.57 (m, 3H), 7.51 (d, J=1.6 Hz, 1H), 7.43 (t, J=5.6 Hz, 2H), 7.39 (t, J=8.0 Hz, 1H), 7.34-7.28 (m, 2H), 4.16 (d, J=2.4 Hz, 2H), 3.99 (s, 2H), 3.91-3.87 (m, 2H), 3.85-3.78 (m, 2H), 3.74-3.66 (m, 2H), 3.65-3.57 (m, 9H), 3.35 (dd, J=8.8 & 1.2 Hz, 1H), 3.0-3.19 (m, 1H), 2.82 (dd, J=2.4 Hz, 1H), 2.78 (dd, J=12.4 & 3.6 Hz, 1H), 1.68 (t, J=11.6 Hz, 1H).

### (2R,4S,5R,6R)-6-((1R,2R)-3-(2-(3-chlorophenyl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 125)

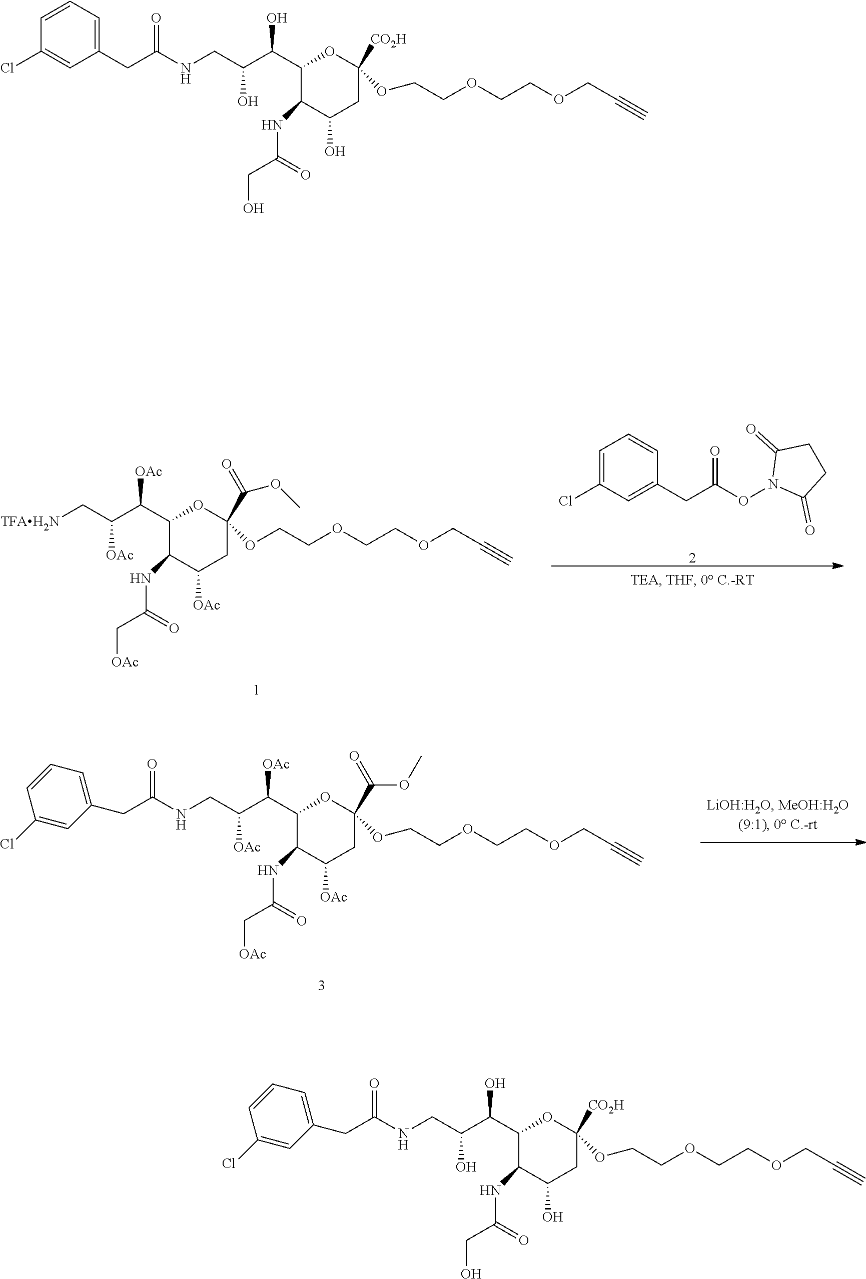

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(3-chlorophenyl)acetamido)propane-1,2-diyl diacetate (3)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-aminopropane-1,2-diyl diacetate (1, 0.400 g, 0.63 mmol) and 2,5-dioxopyrrolidin-1-yl 2-(3-chlorophenyl)acetate (2, 0.070 g, 0.29 mmol) in tetrahydrofuran (4.0 mL), was added trimethyl amine (0.44 mL, 3.164 mmol) at 0° C. then stirred at room temperature for 12 h. After completion the mixture was diluted with water and ethyl acetate. The organic layer was separated, dried, filtered, concentrated, and the crude residue obtained was purified by silica gel column chromatography using ethyl acetate in hexane as eluents to afford the desired product (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(3-chlorophenyl)acetamido)propane-1,2-diyl diacetate (3). Yield: 0.30 g, 60%; LCMS (ESI) m/z 782.70[M−H]⁻.

### Synthesis of (2R,4S,5R,6R)-6-((1R,2R)-3-(2-(3-chlorophenyl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 125)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(3-chlorophenyl)acetamido)propane-1,2-diyl diacetate (3, 0.300 g, 0.38 mmol) in methanol (3.0 mL), was added a solution of lithium hydroxide monohydrate (0.096 g, 2.29 mmol) in water (0.30 mL). The reaction mixture was stirred at room temperature for 6 h. After completion, the reaction mixture was treated with acidic resin (Dowex 50H⁺) up to pH ˜6 and the suspension was filtered. The filtrate was concentrated, and the crude residue obtained was purified by prep HPLC using acetonitrile in water (+0.1% TFA) as eluents to afford the desired product (2R,4S,5R,6R)-6-((1R,2R)-3-(2-(3-chlorophenyl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 125). Yield: 0.017 g, 7%; LCMS m/z 603.35 [M+H]+; ¹H NMR (400 MHz, methanol-d₄) δ 7.34 (s, 1H), 7.31-7.22 (m, 3H), 4.18 (d, J=2.4 Hz, 2H), 4.01 (s, 2H), 3.92-3.78 (m, 4H), 3.73-3.71 (m, 1H), 3.68-3.64 (m, 7H), 3.61-3.58 (m, 1H), 3.53 (s, 2H), 3.37 (dd, J=8.8 & 1.6 Hz, 1H), 3.25-3.19 (m, 1H), 2.83 (dd, J=2.4 Hz, 1H), 2.74 (dd, J=12.8 & 4.0 Hz, 1H), 1.74 (t, J=12.4 Hz, 1H).

### Methyl (2R,4S,5R,6R)-6-((1S,2R)-3-(2-([1,1′-biphenyl]-4-yl)acetamido)-1-fluoro-2-hydroxypropyl)-4-hydroxy-5-(2-hydroxyacetamido)-2-(2-(2-(prop-2-yn-1-yloxy)ethoxy)ethoxy)tetrahydro-2H-pyran-2-carboxylate (Compound 32)

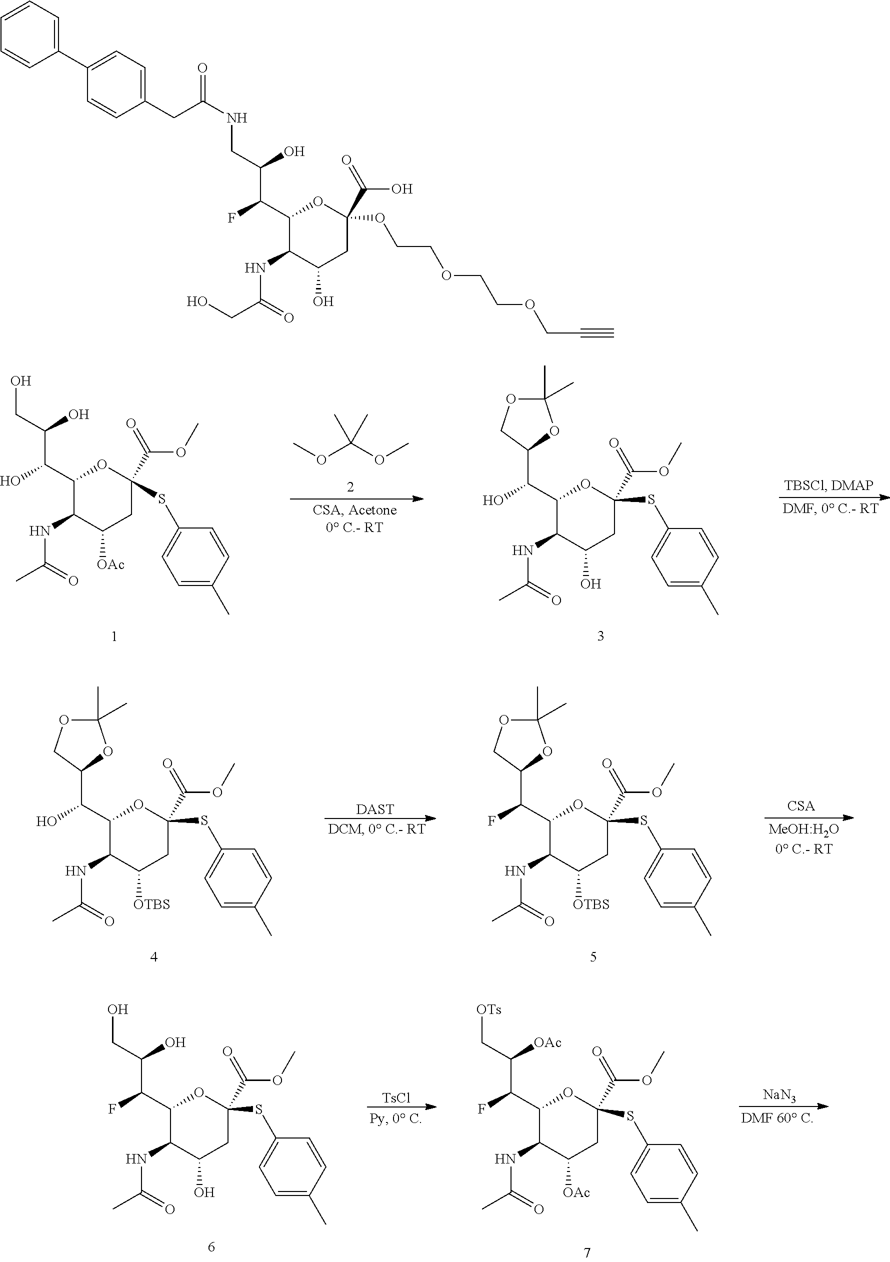

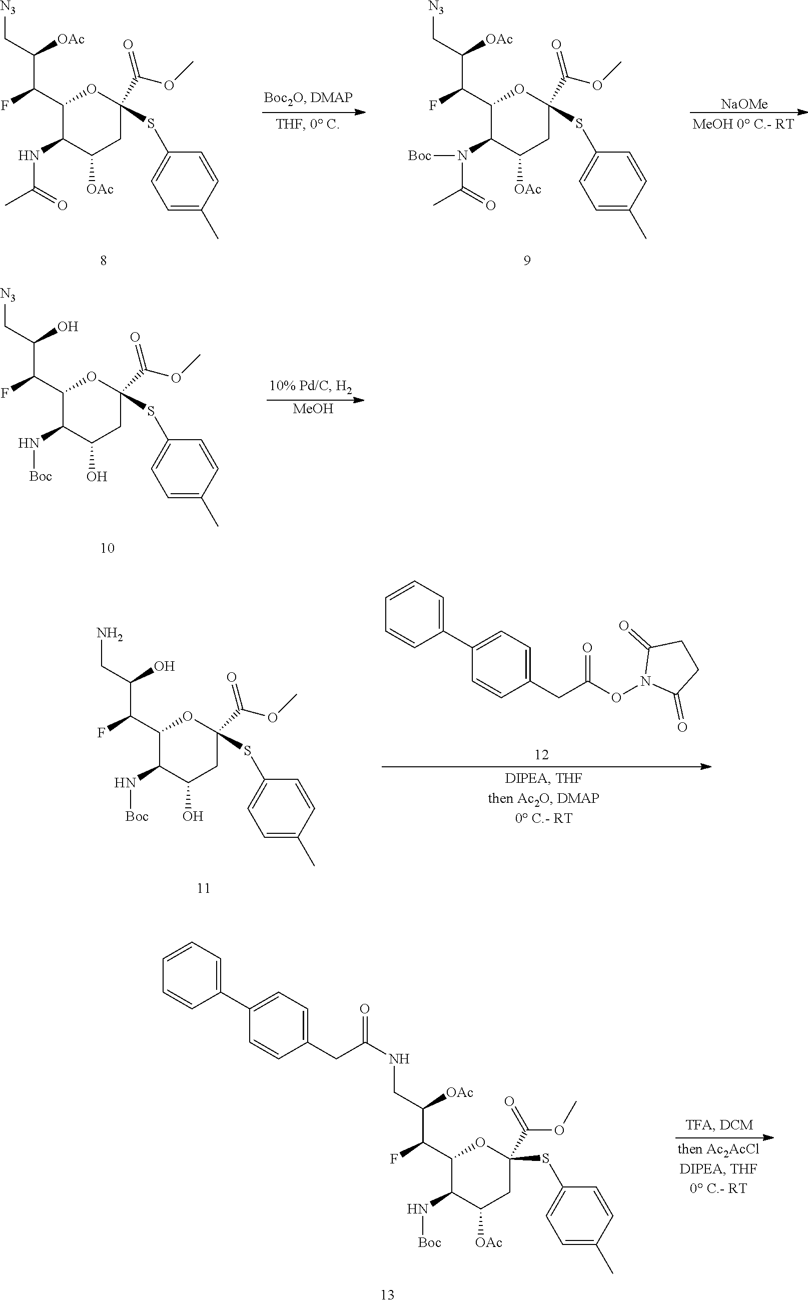

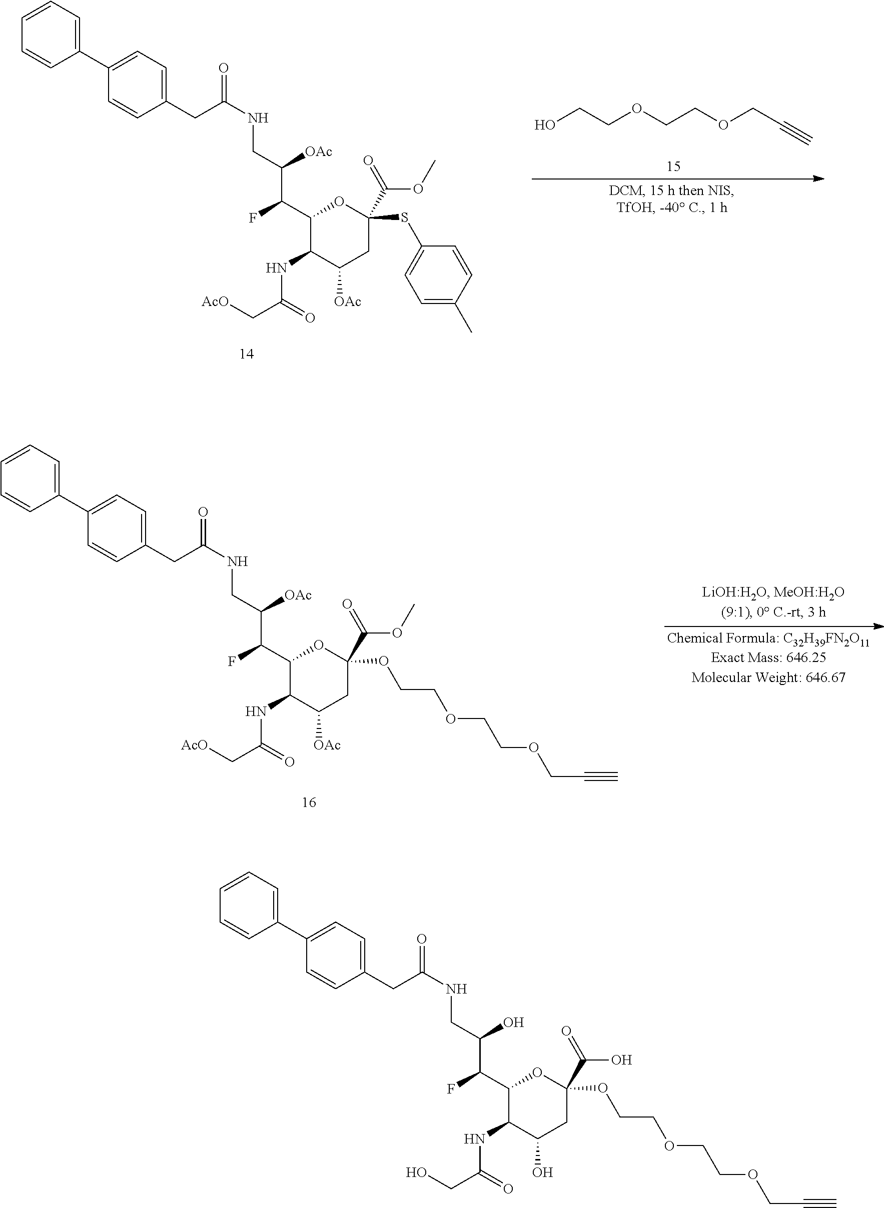

### Synthesis of methyl (2R,4S,5R,6R)-5-acetamido-6-((S)-((R)-2,2-dimethyl-1,3-dioxolan-4-yl)(hydroxy)methyl)-4-hydroxy-2-(p-tolylthio)tetrahydro-2H-pyran-2-carboxylate (3)

To a stirred solution of methyl (2R,4S,5R,6R)-5-acetamido-4-hydroxy-2-(p-tolylthio)-6-((1R,2R)-1,2,3-trihydroxypropyl)tetrahydro-2H-pyran-2-carboxylate (1, 10.0 g, 1 eq, 23.3 mmol) in anhydrous acetone (100 mL), was added 2,2-dimethoxypropane (2, 22.8 g, 8 eq, 186 mmol). The mixture was stirred overnight from 0° C. to room temperature. The reaction mixture was neutralized by addition of triethylamine and concentrated. The crude residue obtained was purified by silica gel column chromatography using ethyl acetate in hexane as eluents to afford the desired product methyl (2R,4S,5R,6R)-5-acetamido-6-((S)-((R)-2,2-dimethyl-1,3-dioxolan-4-yl)(hydroxy)methyl)-4-hydroxy-2-(p-tolylthio)tetrahydro-2H-pyran-2-carboxylate (3). Yield: 8.0 g, 73.17%); LCMS (ESI) m/z 470.2 [M+H]⁺.

### Synthesis of methyl (2R,4S,5S,6R)-5-acetamido-4-((tert-butyldimethylsilyl)oxy)-6-((S)-((R)-2,2-dimethyl-1,3-dioxolan-4-yl)(hydroxy)methyl)-2-(p-tolylthio)tetrahydro-2H-pyran-2-carboxylate (4)

To a stirred solution of methyl (2R,4S,5R,6R)-5-acetamido-6-((S)-((R)-2,2-dimethyl-1,3-dioxolan-4-yl)(hydroxy)methyl)-4-hydroxy-2-(p-tolylthio)tetrahydro-2H-pyran-2-carboxylate (3, 8.0 g, 17.04 mmol) in dry DMF (40 mL) were added tert-butyldimethylsilylchloride (3.85 g, 25.56 mmol) and imidazole (6.24 g, 51.11 mmol). The reaction mixture was stirred for 20 h at room temperature. Upon completion, the reaction mixture was diluted with ethyl acetate (80 ml) and washed with ice-cold water (3×40 mL), saturated aq. NaHCO₃ (40 mL), and water (40 mL) again. The organic phase was separated, dried and concentrated. The residue was purified by silica gel column chromatography using ethyl acetate in hexane to afford the desired product methyl (2R,4S,5S,6R)-5-acetamido-4-((tert-butyldimethylsilyl)oxy)-6-((S)-((R)-2,2-dimethyl-1,3-dioxolan-4-yl)(hydroxy)methyl)-2-(p-tolylthio)tetrahydro-2H-pyran-2-carboxylate (4). Yield: 8.0 g, 80.43%; LCMS (ESI) m/z 583.95 [M+H]⁺.

### Synthesis of methyl (2R,4S,5S,6R)-5-acetamido-4-((tert-butyldimethylsilyl)oxy)-6-((R)-((R)-2,2-dimethyl-1,3-dioxolan-4-yl)fluoromethyl)-2-(p-tolylthio)tetrahydro-2H-pyran-2-carboxylate (5)

To a stirred solution of methyl (2R,4S,5S,6R)-5-acetamido-4-((tert-butyldimethylsilyl)oxy)-6-((S)-((R)-2,2-dimethyl-1,3-dioxolan-4-yl)(hydroxy)methyl)-2-(p-tolylthio)tetrahydro-2H-pyran-2-carboxylate (4, 8.0 g, 13.70 mmol) in DCM (80 mL) was added diethyl(trifluoro-λ⁴-sulfanyl)amine (3.31 mL, 20.55 mmol) at 0° C. then stirred at room temperature for 2 h. After completion, the reaction was quenched with saturated NaHCO₃ then extracted with DCM. The organic layer was concentrated, and the crude was purified by silica gel column chromatography using ethyl acetate in hexane as eluents to afford the desired product methyl (2R,4S,5S,6R)-5-acetamido-4-((tert-butyldimethylsilyl)oxy)-6-((R)-((R)-2,2-dimethyl-1,3-dioxolan-4-yl)fluoromethyl)-2-(p-tolylthio)tetrahydro-2H-pyran-2-carboxylate (5). Yield: 2.10 g, 26.16%; LCMS (ESI) m/z 583.95 [M−H]⁻.

### Synthesis of methyl (2R,4S,5R,6R)-5-acetamido-6-((1S,2R)-1-fluoro-2,3-dihydroxypropyl)-4-hydroxy-2-(p-tolylthio)tetrahydro-2H-pyran-2-carboxylate (6)

To a stirred solution of methyl (2R,4S,5S,6R)-5-acetamido-4-((tert-butyldimethylsilyl)oxy)-6-((R)-((R)-2,2-dimethyl-1,3-dioxolan-4-yl)fluoromethyl)-2-(p-tolylthio)tetrahydro-2H-pyran-2-carboxylate (5, 2.10 g, 3.58 mmol) in methanol (18.0 mL) and water (2.0 mL) was added [(1R,4S)-7,7-dimethyl-2-oxobicyclo[2.2.1]heptan-1-yl]methanesulfonate (2.50 g, 10.75 mmol) at 0° C. then stirred at room temperature for 6 h. After completion, the reaction was quenched with triethylamine then concentrated to afford the desired product methyl (2R,4S,5R,6R)-5-acetamido-6-((1S,2R)-1-fluoro-2,3-dihydroxypropyl)-4-hydroxy-2-(p-tolylthio)tetrahydro-2H-pyran-2-carboxylate (6). Yield: 1.50 g (crude); LCMS (ESI) m/z 432.00 [M+H]⁺. The crude was used for the following step without further purification.

### Synthesis of methyl (2R,4S,5R,6R)-5-acetamido-4-acetoxy-6-((1R,2R)-2-acetoxy-1-fluoro-3-(tosyloxy)propyl)-2-(p-tolylthio)tetrahydro-2H-pyran-2-carboxylate (7)

To a stirred solution of methyl (2R,4S,5R,6R)-5-acetamido-6-((1S,2R)-1-fluoro-2,3-dihydroxypropyl)-4-hydroxy-2-(p-tolylthio)tetrahydro-2H-pyran-2-carboxylate (6, 1.50 g, 3.48 mmol) in pyridine (15.0 mL) was added a solution of 4-methylbenzene-1-sulfonyl chloride (6, 1.19 g, 6.26 mmol) in pyridine (2 mL) drop wise at 0° C. and stirred overnight. After completion, the reaction mixture was cooled to 0° C. and acetic anhydride (1.31 mL, 13.91 mmol) was added. After completion, the mixture was concentrated. The crude residue obtained was purified by silica gel column chromatography using ethyl acetate in hexane as eluents to afford the desired product methyl (2R,4S,5R,6R)-5-acetamido-4-acetoxy-6-((1R,2R)-2-acetoxy-1-fluoro-3-(tosyloxy)propyl)-2-(p-tolylthio)tetrahydro-2H-pyran-2-carboxylate (7). Yield: 1.0 g, 42.9%. LC-MS (ESI) m/z 669.95 [M+H]⁺.

### Synthesis of methyl (2R,4S,5R,6R)-5-acetamido-4-acetoxy-6-((1S,2R)-2-acetoxy-3-azido-1-fluoropropyl)-2-(p-tolylthio)tetrahydro-2H-pyran-2-carboxylate (8)

To a stirred solution of methyl (2R,4S,5R,6R)-5-acetamido-4-acetoxy-6-((1R,2R)-2-acetoxy-1-fluoro-3-(tosyloxy)propyl)-2-(p-tolylthio)tetrahydro-2H-pyran-2-carboxylate (7, 1.0 g, 1.49 mmol) in anhydrous N,N-dimethylformamide (10.0 mL) was added sodium azide (0.485 g, 7.47 mmol) at room temperature was stirred at 60° C. for 10 h. After completion, the reaction mixture was concentrated. The crude residue obtained was purified by silica gel column chromatography using ethyl acetate in hexane as eluents to afford the desired product methyl (2R,4S,5R,6R)-5-acetamido-4-acetoxy-6-((1S,2R)-2-acetoxy-3-azido-1-fluoropropyl)-2-(p-tolylthio)tetrahydro-2H-pyran-2-carboxylate (8). Yield: 0.765 g, 94.7%; LCMS (ESI) m/z 540.95 [M+H]⁺.

### Synthesis of methyl (2R,4S,5R,6R)-4-acetoxy-6-((1S,2R)-2-acetoxy-3-azido-1-fluoropropyl)-5-(N-(tert-butoxycarbonyl)acetamido)-2-(p-tolylthio)tetrahydro-2H-pyran-2-carboxylate (9)

A mixture of methyl (2R,4S,5R,6R)-5-acetamido-4-acetoxy-6-((1S,2R)-2-acetoxy-3-azido-1-fluoropropyl)-2-(p-tolylthio)tetrahydro-2H-pyran-2-carboxylate (8, 0.76 g, 22.0 mmol) in tetrahydrofuran (10 mL) was added 4 Å molecular sieves and the mixture was stirred for 1 h. Di-tert-butyl dicarbonate (1.61 mL; 7.03 mmol) and 4-dimethylaminopyridine (0.343 g, 2.81 mmol) were added was stirred for 5 min at room temperature then 12 h at 65° C. The reaction mixture was filtered, and the filtrate was concentrated. The residue was dissolved in ethyl acetate and the organic layer was washed with aq. sat. sodium chloride, dried, filtered, and the filtrate was concentrated. The crude residue obtained was purified by silica gel column chromatography using ethyl acetate in hexane as eluents to afford the desired product methyl (2R,4S,5R,6R)-4-acetoxy-6-((1S,2R)-2-acetoxy-3-azido-1-fluoropropyl)-5-(N-(tert-butoxycarbonyl)acetamido)-2-(p-tolylthio)tetrahydro-2H-pyran-2-carboxylate (9). Yield: 0.84 g, 93.2%; LCMS m/z 658.20 [M+H₃O]⁺.

### Synthesis of methyl (2R,4S,5R,6R)-6-((1S,2R)-3-azido-1-fluoro-2-hydroxypropyl)-5-((tert-butoxycarbonyl)amino)-4-hydroxy-2-(p-tolylthio)tetrahydro-2H-pyran-2-carboxylate (10)

To a stirred solution of methyl (2R,4S,5R,6R)-4-acetoxy-6-((1S,2R)-2-acetoxy-3-azido-1-fluoropropyl)-5-(N-(tert-butoxycarbonyl)acetamido)-2-(p-tolylthio)tetrahydro-2H-pyran-2-carboxylate (9, 0.84 g; 1.31 mmol) in methanol (10 mL) at 0° C. was added 25% sodium methoxide in methanol (0.029 mL, 0.131 mmol, pH should not be more than 9). The reaction mixture was stirred for 2 h at room temperature then neutralized with acidic resin (Dowex-Hydrogen form) until pH˜6. The suspension was filtered, and the filtrate was concentrated to afford the desired product methyl (2R,4S,5R,6R)-6-((1S,2R)-3-azido-1-fluoro-2-hydroxypropyl)-5-((tert-butoxycarbonyl)amino)-4-hydroxy-2-(p-tolylthio)tetrahydro-2H-pyran-2-carboxylate (10) as a crude which was used for next step without further purification. Yield: 0.65 g, Crude; LCMS m/z 514.90 [M+H]⁺.

### Synthesis of methyl (2R,4S,5R,6R)-6-((1S,2R)-3-amino-1-fluoro-2-hydroxypropyl)-5-((tert-butoxycarbonyl)amino)-4-hydroxy-2-(p-tolylthio)tetrahydro-2H-pyran-2-carboxylate (11)

To a stirred solution of methyl (2R,4S,5R,6R)-6-((1S,2R)-3-azido-1-fluoro-2-hydroxypropyl)-5-((tert-butoxycarbonyl)amino)-4-hydroxy-2-(p-tolylthio)tetrahydro-2H-pyran-2-carboxylate (10, 0.65 g, 1.26 mmol) in methanol (10 mL) was added 10% Pd/C (0.65 g, 100% w/w) at room temperature. The reaction was then hydrogenated under 1 atm of H₂ gas for 12 h. After completion, the reaction was filtered through celite, and the filtrate was concentrated. The crude residue was dried under vacuum to afford the desired product methyl (2R,4S,5R,6R)-6-((1S,2R)-3-amino-1-fluoro-2-hydroxypropyl)-5-((tert-butoxycarbonyl)amino)-4-hydroxy-2-(p-tolylthio)tetrahydro-2H-pyran-2-carboxylate (11) as a crude. Yield (0.60 g, crude); LCMS, m/z 489.25 [M+H]⁺.

### Synthesis of methyl (2R,4S,5R,6R)-6-((1S,2R)-3-(2-([1,1′-biphenyl]-4-yl)acetamido)-2-acetoxy-1-fluoropropyl)-4-acetoxy-5-((tert-butoxycarbonyl)amino)-2-(p-tolylthio)tetrahydro-2H-pyran-2-carboxylate (13)

To a stirred solution of methyl (2R,4S,5R,6R)-6-((1S,2R)-3-amino-1-fluoro-2-hydroxypropyl)-5-((tert-butoxycarbonyl)amino)-4-hydroxy-2-(p-tolylthio)tetrahydro-2H-pyran-2-carboxylate (11, 0.60 g, 1.23 mmol) in tetrahydrofuran (10.0 mL) was added ethylbis(propan-2-yl)amine (0.818 mL, 6.14 mmol) was added 2,5-dioxopyrrolidin-1-yl 2-([1,1′-biphenyl]-4-yl)acetate (0.341 g, 1.11 mmol) at 0° C. then stirred at room temperature for 12 h. After completion, the reaction mixture was cooled to 0° C. and acetic anhydride (0.464 mL, 4.91 mmol) and DMAP (0.075 g, 0.614 mmol) were added. The reaction was stirred at room temperature for another 6 h then concentrated. The crude residue obtained was purified by silica gel column chromatography using ethyl acetate in hexane as eluents to afford the desired product methyl (2R,4S,5R,6R)-6-((1S,2R)-3-(2-([1,1′-biphenyl]-4-yl)acetamido)-2-acetoxy-1-fluoropropyl)-4-acetoxy-5-((tert-butoxycarbonyl)amino)-2-(p-tolylthio)tetrahydro-2H-pyran-2-carboxylate (13). Yield: 0.76 g, 80.70%; LCMS (ESI) m/z 764.85 [M−H]⁻.

### Synthesis of methyl (2R,4S,5R,6R)-6-((1S,2R)-3-(2-([1,1′-biphenyl]-4-yl)acetamido)-2-acetoxy-1-fluoropropyl)-4-acetoxy-5-(2-acetoxyacetamido)-2-(p-tolylthio)tetrahydro-2H-pyran-2-carboxylate (14)

To a stirred solution of methyl (2R,4S,5R,6R)-6-((1S,2R)-3-(2-([1,1′-biphenyl]-4-yl)acetamido)-2-acetoxy-1-fluoropropyl)-4-acetoxy-5-((tert-butoxycarbonyl)amino)-2-(p-tolylthio)tetrahydro-2H-pyran-2-carboxylate (13, 0.75 g, 0.977 mmol) in dichloromethane (5 mL), was added trifluoroacetic acid (0.36 g, 50% w/w) dropwise at 0° C. The reaction mixture was stirred at room temperature for 3 h. After completion, the reaction mixture was concentrated, and azeotropped with toluene (3-4 times)

To a mixture of the residue previously obtained methyl (2R,4S,5R,6R)-6-((1S,2R)-3-(2-([1,1′-biphenyl]-4-yl)acetamido)-2-acetoxy-1-fluoropropyl)-4-acetoxy-5-amino-2-(p-tolylthio)tetrahydro-2H-pyran-2-carboxylate (0.65 g, 0.974 mmol) in tetrahydrofuran (10 mL), was added ethylbis(propan-2-yl)amine (0.389 mL, 2.92 mmol) dropwise at 0° C. After 10 min 2-chloro-2-oxoethyl acetate (0.108 mL, 0.877 mmol) was added and the mixture was stirred at room temperature for 1 h. After completion, the reaction mixture was washed with saturated brine solution and extracted with dichloromethane. The organic layer was dried, filtered, and concentrated. The crude residue was purified by silica gel column chromatography using ethyl acetate in hexane as eluents to afford the desired product methyl (2R,4S,5R,6R)-6-((1S,2R)-3-(2-([1,1′-biphenyl]-4-yl)acetamido)-2-acetoxy-1-fluoropropyl)-4-acetoxy-5-(2-acetoxyacetamido)-2-(p-tolylthio)tetrahydro-2H-pyran-2-carboxylate (14). Yield: 0.45 g, 60.20%; ELSD-MS m/z 767.30 [M+H]⁺.

### Synthesis of methyl (2R,4S,5R,6R)-6-((1S,2R)-3-(2-([1,1′-biphenyl]-4-yl)acetamido)-2-acetoxy-1-fluoropropyl)-4-acetoxy-5-(2-acetoxyacetamido)-2-(2-(2-(prop-2-yn-1-yloxy)ethoxy)ethoxy)tetrahydro-2H-pyran-2-carboxylate (16)

A mixture of methyl (2R,4S,5R,6R)-6-((1S,2R)-3-(2-([1,1′-biphenyl]-4-yl)acetamido)-2-acetoxy-1-fluoropropyl)-4-acetoxy-5-(2-acetoxyacetamido)-2-(p-tolylthio)tetrahydro-2H-pyran-2-carboxylate (14, 0.45 g, 0.586 mmol), 2-(2-(prop-2-yn-1-yloxy)ethoxy)ethan-1-ol (15, 0.211 g, 1.47 mmol) and activated powdered 4 Å molecular sieves (0.45 g, 100% w/w) in anhydrous dichloromethane (15.0 mL) was stirred at room temperature for 15 h. The solution was then cooled to −40° C. followed by addition of 1-iodopyrrolidine-2,5-dione (0.316 g, 1.41 mmol) and trifluoromethanesulfonic acid (0.072 mL, 0.586 mmol). The reaction mixture was stirred at the same temperature for another 2 h. After completion, the reaction mixture was quenched by addition of triethylamine (neutral pH) and warmed up gradually to room temperature. The reaction mixture was filtered, and the filtrate was washed with deionized water. The organic layer was separated, dried, filtered, and concentrated. The crude residue obtained was purified by silica gel column chromatography using ethyl acetate in hexane as eluents to afford the desired product methyl (2R,4S,5R,6R)-6-((1S,2R)-3-(2-([1,1′-biphenyl]-4-yl)acetamido)-2-acetoxy-1-fluoropropyl)-4-acetoxy-5-(2-acetoxyacetamido)-2-(2-(2-(prop-2-yn-1-yloxy)ethoxy)ethoxy)tetrahydro-2H-pyran-2-carboxylate (5). Yield: 0.325 g, 83.82%; LCMS m/z 787.80 [M+H]⁺.

### Synthesis of methyl (2R,4S,5R,6R)-6-((1S,2R)-3-(2-([1,1′-biphenyl]-4-yl)acetamido)-1-fluoro-2-hydroxypropyl)-4-hydroxy-5-(2-hydroxyacetamido)-2-(2-(2-(prop-2-yn-1-yloxy)ethoxy)ethoxy)tetrahydro-2H-pyran-2-carboxylate (Cpd. 32)

To a stirred solution of methyl (2R,4S,5R,6R)-6-((1S,2R)-3-(2-([1,1′-biphenyl]-4-yl)acetamido)-2-acetoxy-1-fluoropropyl)-4-acetoxy-5-(2-acetoxyacetamido)-2-(2-(2-(prop-2-yn-1-yloxy)ethoxy)ethoxy)tetrahydro-2H-pyran-2-carboxylate (5, 0.320 g, 0.406 mmol) in methanol (10.0 mL), was added a solution of lithium hydroxide monohydrate (0.0.051 g, 1.22 mmol) in water (1.0 mL). The reaction mixture was stirred at room temperature for 6 h. After completion, the reaction mixture was treated with acidic resin (Dowex 50H⁺) up to pH ˜6 and the suspension was filtered. The filtrate was concentrated, and the crude residue obtained was purified by prep HPLC using acetonitrile in water (+0.1% TFA) as eluents to afford the desired product methyl (2R,4S,5R,6R)-6-((1S,2R)-3-(2-([1,1′-biphenyl]-4-yl)acetamido)-1-fluoro-2-hydroxypropyl)-4-hydroxy-5-(2-hydroxyacetamido)-2-(2-(2-(prop-2-yn-1-yloxy)ethoxy)ethoxy)tetrahydro-2H-pyran-2-carboxylate (Cpd. 32). Yield: 0.025 g, 9.30%; LCMS m/z 647.40 [M+H]+; ¹H NMR (400 MHz, methanol-d4) (7.62-7.57 (m, 4H), 7.44-7.38 (m, 4H), 7.33-7.30 (m, 1H), 4.57 (dd, J=47.4 & 4.2 Hz, 1H), 4.18-4.08 (m, 3H), 3.97 (d, J=1.2 Hz, 2H), 3.93-3.87 (m, 2H), 3.83-3.73 (m, 2H), 3.62-3.54 (m, 10H), 3.45-3.40 (m, 2H), 2.83 (t, J=2.4 Hz, 1H), 2.70 (dd, J=12.8 & 4.4 Hz, 1H), 1.74 (t, J=12.0 Hz, 1H).

### (2R,4S,5R,6R)-6-((1R,2R)-3-(2-(3-chloro-4-hydroxyphenyl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 197)

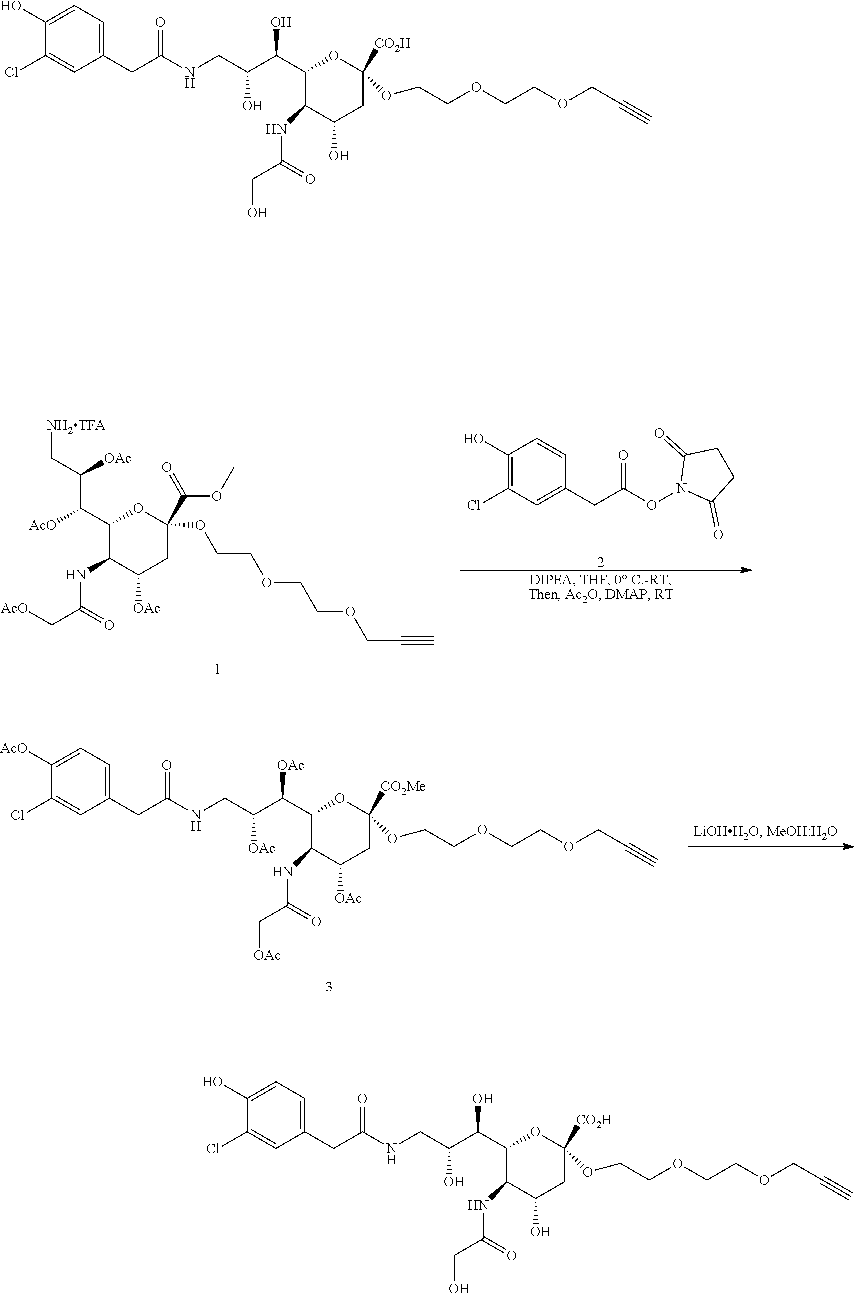

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(4-acetoxy-3-chlorophenyl)acetamido)propane-1,2-diyl diacetate (3)

To a solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-((2,2,2-trifluoroacetyl)-14-azaneyl)propane-1,2-diyl diacetate (1, 0.30 g, 0.41 mmol) and 2,5-dioxopyrrolidin-1-yl 2-(3-chloro-4-hydroxyphenyl)acetate (2, 0.268 g, 0.95 mmol) in tetrahydrofuran (5.0 mL) was added ethylbis(propan-2-yl)amine (0.413 mL, 2.73 mmol) at 0° C. then stirred at room temperature for 4 h. Acetic anhydride (0.134 mL, 1.42 mmol) and 4-dimethylaminopyridine (0.058 g, 0.474 mmol) were added at 0° C. and the resulting mixture was stirred at room temperature for 12 h. After completion the mixture was diluted with water and ethyl acetate. The organic layer was separated, dried, filtered, concentrated, and the crude residue obtained was purified by silica gel column chromatography using ethyl acetate in hexane as eluents to afford the desired product (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(4-acetoxy-3-chlorophenyl)acetamido)propane-1,2-diyl diacetate (3). Yield: 0.150 g, 37.5%; LCMS (ESI) m/z 843.00 [M+H]⁺.

### Synthesis of (2R,4S,5R,6R)-6-((1R,2R)-3-(2-(3-chloro-4-hydroxyphenyl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 197)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(4-acetoxy-3-chlorophenyl)acetamido)propane-1,2-diyl diacetate (3, 0.150 g, 0.18 mmol) in methanol (4.0 mL), was added a solution of lithium hydroxide monohydrate (0.045 g, 1.06 mmol) in water (0.30 mL). The reaction mixture was stirred at room temperature for 6 h. After completion, the reaction mixture was treated with acidic resin (Dowex 50H⁺) up to pH ˜6 and the suspension was filtered. The filtrate was concentrated, and the crude residue obtained was purified by prep HPLC using acetonitrile in water (+0.1% TFA) as eluents to afford the desired product (2R,4S,5R,6R)-6-((1R,2R)-3-(2-(3-chloro-4-hydroxyphenyl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (197). Yield: 0.012 g, 10%; LCMS m/z 619.2 [M+H]+; ¹H NMR (400 MHz, methanol-d₄) δ 7.25 (d, J=2.0 Hz, 1H), 7.06-7.04 (m, 1H), 6.85 (d, J=8.0 Hz, 1H), 4.19 (d, J=2.4 Hz, 2H), 4.01 (brs, 2H), 3.92-3.59 (m, 13H), 3.42 (s, 2H), 3.36-3.31 (m, 1H), 3.22-3.16 (m, 1H), 2.83 (t, J=2.4 Hz, 1H), 2.76-2.73 (dd, J=8.8 Hz, 13.2 Hz, 1H), 1.74 (t, J=12.0 Hz, 1H).

### (2R,4S,5R,6R)-6-((1R,2R)-1,2-dihydroxy-3-(2-(2-hydroxyphenyl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 151)

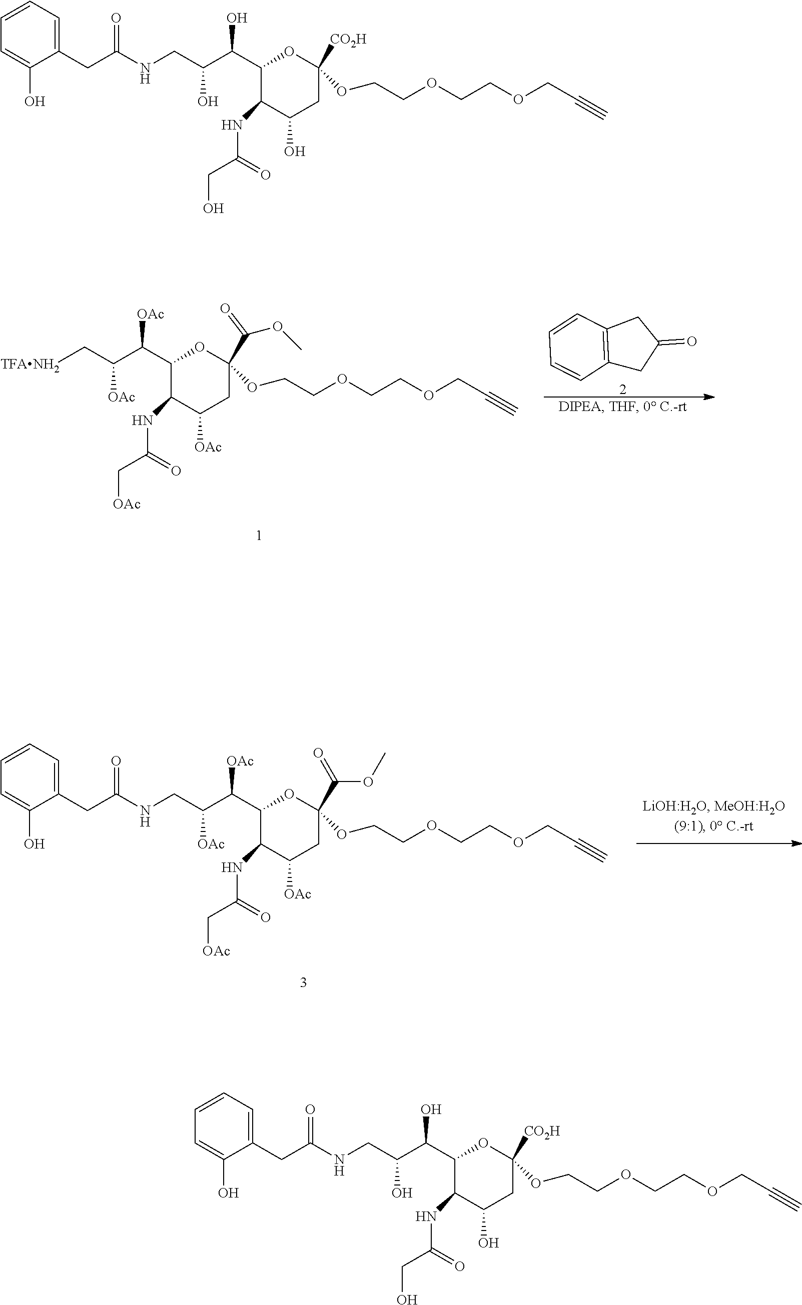

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(2-hydroxyphenyl)acetamido)propane-1,2-diyl diacetate (3)

To a solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-aminopropane-1,2-diyl diacetate (1, 0.400 g, 0.63 mmol), in tetrahydrofuran (5.0 mL), ware added ethylbis(propan-2-yl)amine (0.330 mL, 1.89 mmol) and benzofuran-2(3H)-one (2, 0.848 g, 0.63 mmol) at 0° C. then stirred at room temperature for 6 h. After completion the mixture was concentrated, and the crude residue obtained was purified by silica gel column chromatography using ethyl acetate in hexane as eluents to afford the desired product (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(2-hydroxyphenyl)acetamido)propane-1,2-diyl diacetate (3). Yield: 0.250 g, 51%; LCMS (ESI) m/z 767.10 [M+H]⁺.

### Synthesis of (2R,4S,5R,6R)-6-((1R,2R)-1,2-dihydroxy-3-(2-(2-hydroxyphenyl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 151)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(2-hydroxyphenyl)acetamido)propane-1,2-diyl diacetate (3, 0.250 g, 0.32 mmol) in methanol (5.0 mL), was added a solution of lithium hydroxide monohydrate (0.046 g, 1.92 mmol) in water (0.5 mL). The reaction mixture was stirred at room temperature for 6 h. After completion, the reaction mixture was treated with acidic resin (Dowex 50H⁺) up to pH ˜6 and the suspension was filtered. The filtrate was concentrated, and the crude residue obtained was purified by prep HPLC using acetonitrile in water (+0.1% TFA) as eluents to afford the desired product (2R,4S,5R,6R)-6-((1R,2R)-1,2-dihydroxy-3-(2-(2-hydroxyphenyl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 151). Yield: 0.017 g, 9%; LCMS m/z 585.30 [M+H]+; ¹H NMR (400 MHz, methanol-d₄) δ 7.13-7.07 (m, 2H), 6.81-6.77 (m, 2H), 4.18 (d, J=2.4 Hz, 2H), 4.01 (s, 2H), 3.93-3.77 (m, 4H), 3.72-3.69 (m, 1H), 3.68-3.62 (m, 5H), 3.61-3.57 (m, 3H), 3.54 (s, 2H), 3.45 (dd, J=12.0 & 1.2 Hz, 1H), 3.23 (dd, J=7.6 & 1.2 Hz, 1H), 2.83 (t, J=2.4 Hz, 1H), 2.73 (dd, J=12.4 & 4.0 Hz, 1H), 1.73 (t, J=12.4 Hz, 1H).

### (2R,4S,5R,6R)-6-((1R,2R)-3-(2-(4-chloro-3-hydroxyphenyl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 134)

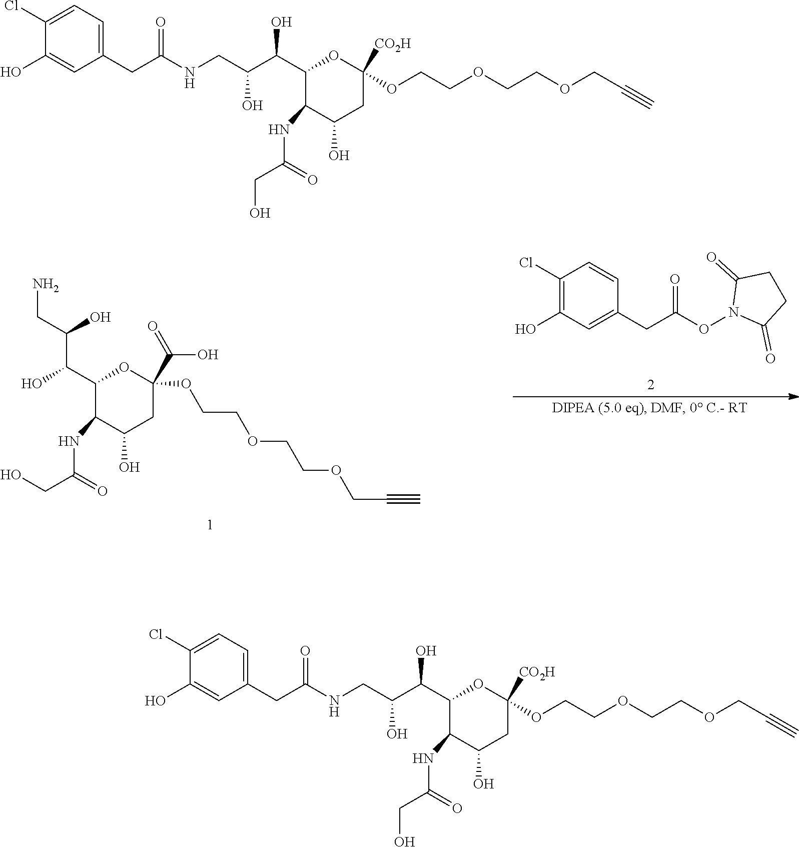 [SMILES: C#CCOCCOCCO[C@]1(C(=O)O)C[C@H](O)[C@@H](NC(=O)CO)[C@H]([C@H](O)[C@H](O)CNC(=O)Cc2ccc(Cl)c(O)c2)O1 ; C#CCOCCOCCO[C@]1(C(=O)O)C[C@H](O)[C@@H](NC(=O)CO)[C@H]([C@H](O)[C@H](O)CN)O1 ; O=C(Cc1ccc(Cl)c(O)c1)ON1C(=O)CCC1=O ; C#CCOCCOCCO[C@]1(C(=O)O)C[C@H](O)[C@@H](NC(=O)CO)[C@H]([C@H](O)[C@H](O)CNC(=O)Cc2ccc(Cl)c(O)c2)O1]

### Synthesis of (2R,4S,5R,6R)-6-((1R,2R)-3-(2-(4-chloro-3-hydroxyphenyl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 134)

To a stirred solution of (2R,4S,5R,6R)-6-((1R,2R)-3-amino-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (1, 0.30 g, 0.666 mmol) and 2,5-dioxopyrrolidin-1-yl 2-(4-chloro-3-hydroxyphenyl)acetate (2, 0.189 g, 0.666 mmol) in N,N-dimethylformamide (5.0 mL) was added ethylbis(propan-2-yl)amine (0.35 mL, 2.00 mmol) at 0° C. then stirred at room temperature for 4 h. After completion, the reaction mixture was concentrated, and the crude residue obtained was purified by prep HPLC using acetonitrile in water (+0.1% FA) to afford the desired product (2R,4S,5R,6R)-6-((1R,2R)-3-(2-(4-chloro-3-hydroxyphenyl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 134). Yield: 0.050 g, 12%; LCMS m/z 619.25 [M+H]+; 1H NMR (400 MHz, methanol-d4) 7.96-7.93 (m, 1H), 7.90 (d, J=7.6 Hz, 1H), 7.21 (d, J=8.0 Hz, 1H), 6.87 (d, J=2.0 Hz, 1H), 6.74 (dd, J=8 Hz & 1.6 Hz, 1H), 4.19 (d, J=2.4 Hz, 2H), 4.01 (s, 2H), 3.91-3.79 (m, 4H), 3.72-3.58 (m, 9H), 3.46 (s, 2H), 3.36 (d, J=8.4 Hz, 1H), 3.25-3.18 (m, 1H), 2.83 (t, J=2.4 Hz, 1H), 2.76 (dd, J=2.8 Hz & 12.4 Hz, 1H), 1.71 (t, J=11.6 Hz, 1H).

### (2R,4S,5R,6R)-6-((1R,2R)-1,2-dihydroxy-3-(2-(2-oxoindolin-6-yl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 12)

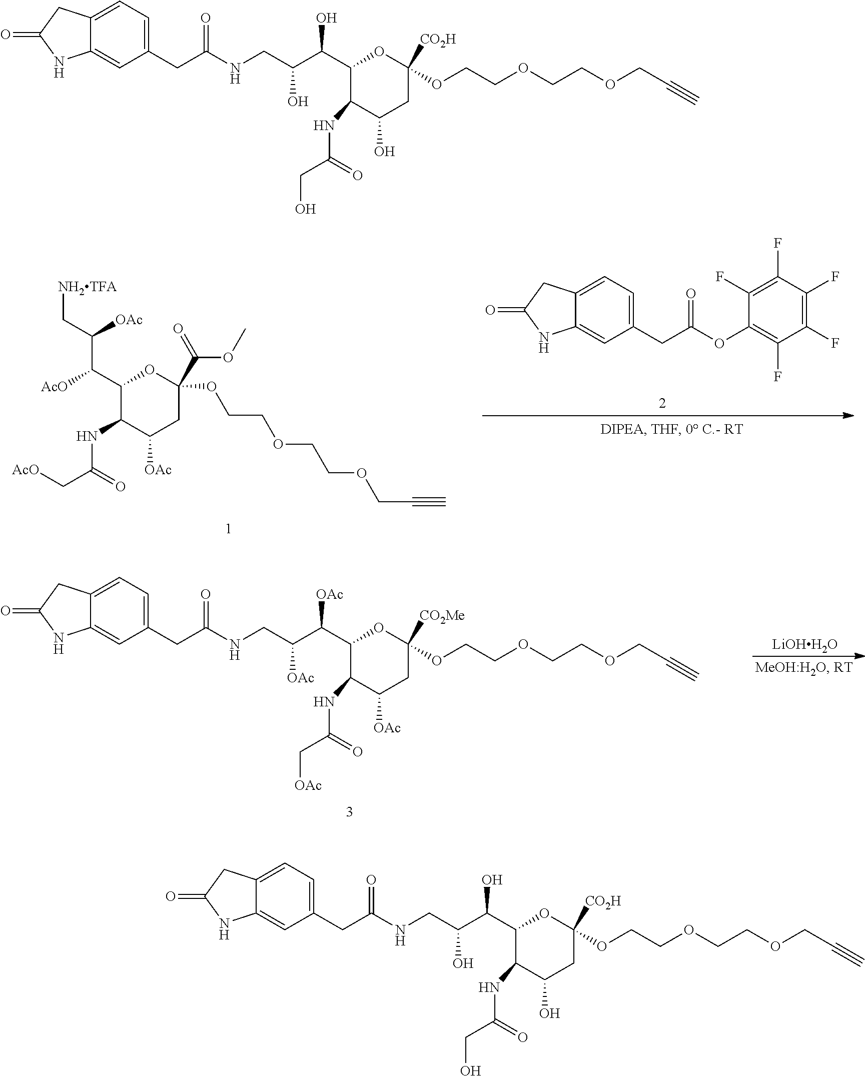

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(2-oxoindolin-6-yl)acetamido)propane-1,2-diyl diacetate (3)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-((2,2,2-trifluoroacetyl)-14-azaneyl)propane-1,2-diyl diacetate (1, 0.40 g, 0.549 mmol) and perfluorophenyl 2-(2-oxoindolin-6-yl)acetate (2, 0.196 g, 0.549 mmol) in tetrahydrofuran (10.0 mL), was added ethylbis(propan-2-yl)amine (0.285 mL, 1.65 mmol) at 0° C. then stirred at room temperature for 2 h. After completion the mixture was diluted with water and ethyl acetate. The organic layer was separated, dried, filtered, concentrated, and the crude residue obtained was purified by silica gel column chromatography and desired compound eluted in ethyl acetate in hexane as eluents to afford the desired product (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(2-oxoindolin-6-yl)acetamido)propane-1,2-diyl diacetate (3). Yield: 0.20 g, 45%; LCMS (ESI) m/z 803.9 [M−H]⁻.

### Synthesis of (2R,4S,5R,6R)-6-((1R,2R)-1,2-dihydroxy-3-(2-(2-oxoindolin-6-yl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 12)

To a stirred solution of methyl (2R,4S,5R,6R)-4-(acetyloxy)-5-[2-(acetyloxy)acetamido]-6-[(1R,2R)-1,2-bis(acetyloxy)-3-[2-(2-oxo-2,3-dihydro-1H-indol-6-yl)acetamido]propyl]-2-{2-[2-(prop-2-yn-1-yloxy)ethoxy]ethoxy}oxane-2-carboxylate (3, 0.250 g, 0.310 mmol) in methanol (5.0 mL), was added a solution of lithium hydroxide monohydrate (0.045 g, 1.68 mmol) in water (0.5 mL). The reaction mixture was stirred at room temperature for 6 h. After completion, the reaction mixture was treated with acidic resin (Dowex 50H⁺) up to pH ˜6 and the suspension was filtered. The filtrate was concentrated, and the crude residue obtained was purified by prep HPLC using acetonitrile in water (+0.1% TFA) as eluents to afford the desired product (2R,4S,5R,6R)-6-((1R,2R)-1,2-dihydroxy-3-(2-(2-oxoindolin-6-yl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 12). Yield: 0.011 g, 9.82%; LCMS m/z 624.40 [M+H]+; ¹H NMR (400 MHz, methanol-d₄) 7.87-7.85 (m, 1H), 7.18 (d, J=7.6 Hz, 1H), 6.93 (d, J=7.6 Hz, 1H), 6.86 (s, 1H), 4.18 (d, J=2.4 Hz, 2H), 4.00 (bs, 2H), 3.91-3.76 (m, 4H), 3.72-3.57 (m, 9H), 3.51 (bs, 2H), 3.49 (bs, 2H), 3.36-3.34 (m, 1H), 3.30-3.22 (m, 1H), 2.83 (t, J=2.4 Hz, 1H), 2.77-2.73 (dd, J=4.0 Hz, 12.4 Hz, 1H), 1.72 (t, J=12.0 Hz, 1H).

### (2R,4S,5R,6R)-6-((1R,2R)-1,2-dihydroxy-3-(2-(2-oxoindolin-5-yl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 11)

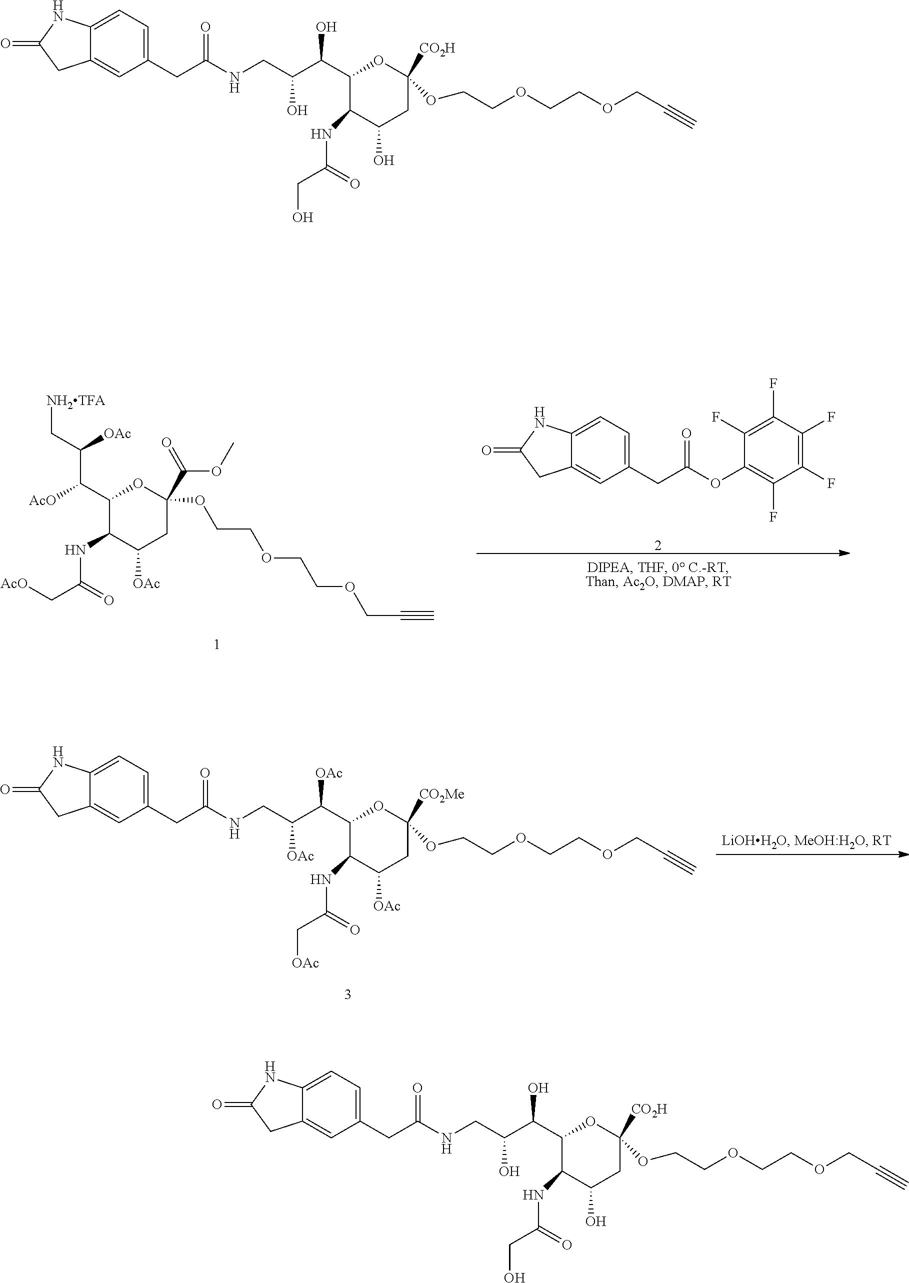

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(2-oxoindolin-5-yl)acetamido)propane-1,2-diyl diacetate (3)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-((2,2,2-trifluoroacetyl)-14-azaneyl)propane-1,2-diyl diacetatee (1, 0.20 g, 0.274 mmol) and perfluorophenyl 2-(2-oxoindolin-5-yl)acetate (2, 0.196 g, 0.549 mmol) in tetrahydrofuran (5.0 mL), was added ethylbis(propan-2-yl)amine (0.237 mL, 1.37 mmol) at 0° C. then stirred at room temperature for 2 h. After completion, the reaction mixture was diluted with water and ethyl acetate. The organic layer was separated, dried, filtered, concentrated, and the crude residue obtained was purified by silica gel column chromatography using in ethyl acetate in hexane as eluents to afford the desired product (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(2-oxoindolin-6-yl)acetamido)propane-1,2-diyl diacetate (3). Yield: 0.230 g, 97%; LCMS (ESI) m/z 803.8 [M−H]⁻.

### Synthesis of (2R,4S,5R,6R)-6-((1R,2R)-1,2-dihydroxy-3-(2-(2-oxoindolin-5-yl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 11)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(2-oxoindolin-5-yl)acetamido)propane-1,2-diyl diacetate (3, 0.230 g, 0.285 mmol) in methanol (5.0 mL), was added a solution of lithium hydroxide monohydrate (0.034 g, 1.43 mmol) in water (0.5 mL). The reaction mixture was stirred at room temperature for 6 h. After completion, the reaction mixture was treated with acidic resin (Dowex 50H⁺) up to pH ˜6 and the suspension was filtered. The filtrate was concentrated, and the crude residue obtained was purified by prep HPLC using acetonitrile in water (+0.1% TFA) as mobile phase to afford the desired product (2R,4S,5R,6R)-6-((1R,2R)-1,2-dihydroxy-3-(2-(2-oxoindolin-5-yl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 11). Yield: 0.028 g, 16%; LCMS m/z 624.35 [M+H]+; ¹H NMR (400 MHz, methanol-d₄) 7.89 (d, J=6.8 Hz, 1H), 7.20 (s, 1H), 7.14 (d, J=7.6 Hz, 1H), 6.84 (d, J=8.0 Hz, 1H), 4.18 (d, J=2.4 Hz, 2H), 4.00 (bs, 2H), 3.91-3.49 (m, 18H), 3.36-3.34 (m, 2H), 3.27-3.20 (m, 1H), 2.84 (t, J=2.4 Hz, 1H), 2.75-2.71 (dd, J=3.6 Hz, 12.4 Hz, 1H), 1.74 (t, J=12.0 Hz, 1H).

### (2R,4S,5R,6R)-6-((1R,2R)-3-(2-(1H-indol-5-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 196)

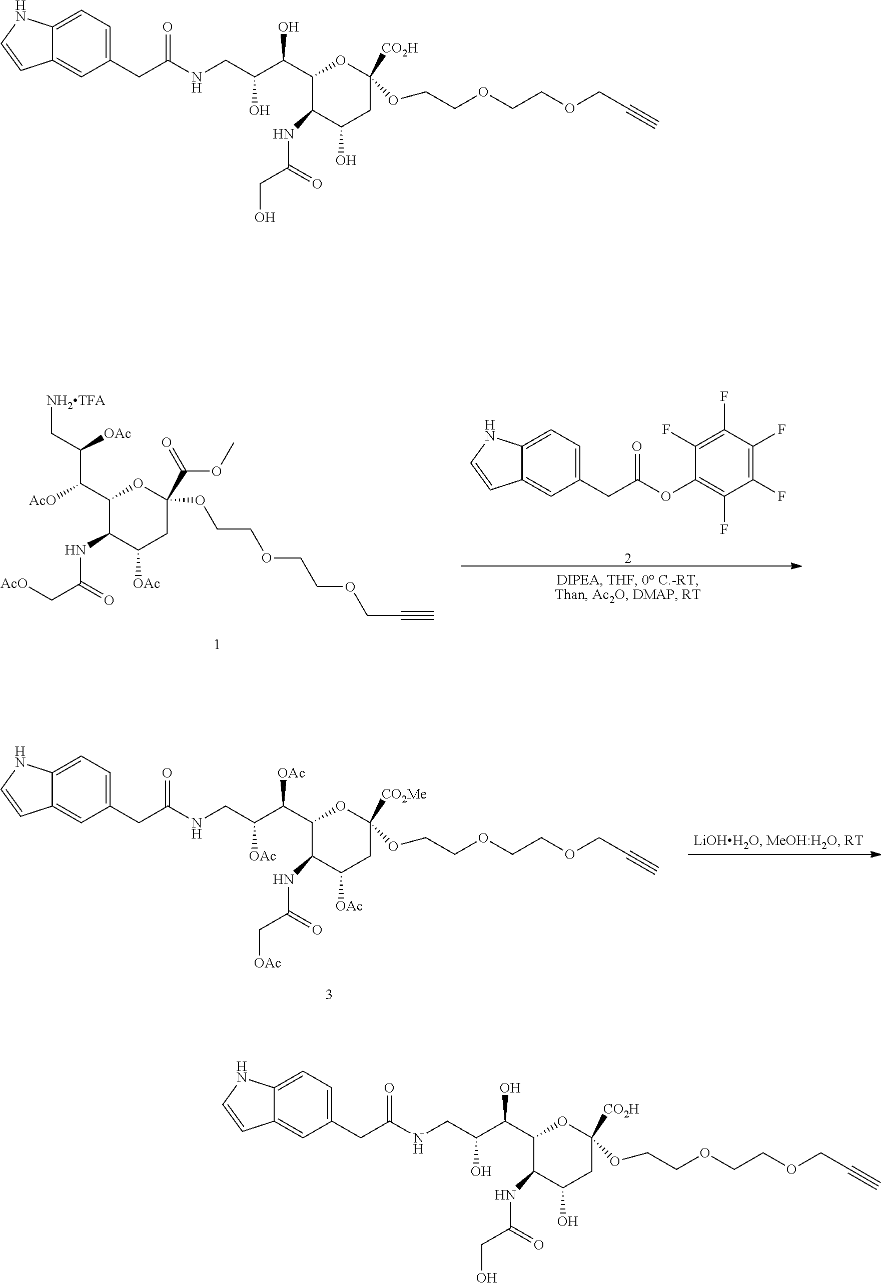

### Synthesis of (1R,2R)-3-(2-(1H-indol-5-yl)acetamido)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)propane-1,2-diyl diacetate (3)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-((2,2,2-trifluoroacetyl)-14-azaneyl)propane-1,2-diyl diacetate (1, 0.40 g, 0.549 mmol) and perfluorophenyl 2-(1H-indol-5-yl)acetate (2, 0.187 g, 0.549 mmol) in tetrahydrofuran (10.0 mL), was added ethylbis(propan-2-yl)amine (0.285 mL, 1.65 mmol) at 0° C. then stirred at room temperature for 2 h. After completion the mixture was diluted with water and ethyl acetate. The organic layer was separated, dried, filtered, concentrated, and the crude residue obtained was purified by silica gel column chromatography and desired compound eluted with ethyl acetate in hexane as eluents to afford the desired product (1R,2R)-3-(2-(1H-indol-5-yl)acetamido)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)propane-1,2-diyl diacetate (3). Yield: 0.400 g, 67%; LCMS (ESI) m/z 787.9 [M−H]⁻.

### Synthesis of (2R,4S,5R,6R)-6-((1R,2R)-3-(2-(1H-indol-5-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 196)

To a stirred solution of (1R,2R)-3-(2-(1H-indol-5-yl)acetamido)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)propane-1,2-diyl diacetate (3, 0.40 g, 0.506 mmol) in methanol (10.0 mL), was added a solution of lithium hydroxide monohydrate (0.073 g, 3.04 mmol) in water (1 mL). The reaction mixture was stirred at room temperature for 6 h. After completion, the reaction mixture was treated with acidic resin (Dowex 50H⁺) up to pH ˜6 and the suspension was filtered. The filtrate was concentrated, and the crude residue obtained was purified by prep HPLC using acetonitrile in water (+0.1% TFA) as eluents to afford the desired product to afford the desired product (2R,4S,5R,6R)-6-((1R,2R)-3-(2-(1H-indol-5-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 196). Yield: 0.045 g, 15%; LCMS m/z 608.35 [M+H]+; ¹H NMR (400 MHz, methanol-d₄) 7.48 (s, 1H), 7.34 (d, J=8.4 Hz, 1H), 7.21-7.20 (m, 1H), 7.05-7.03 (m, 1H), 6.40 (d, J=2.4 Hz, 1H), 4.17 (d, J=2.4 Hz, 2H), 3.99 (bs, 2H), 3.88-3.77 (m, 4H), 3.72-3.55 (m, 11H), 3.35-3.33 (m, 1H), 3.24-3.19 (m, 1H), 2.83 (t, J=2.4 Hz, 1H), 2.72 (dd, J=4.2 Hz, 12.6 Hz, 1H), 1.73 (t, J=12.0 Hz, 1H).

### (2R,4S,5R,6R)-6-((1R,2R)-3-(2-(4-(1H-indol-5-yl)phenyl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 4)

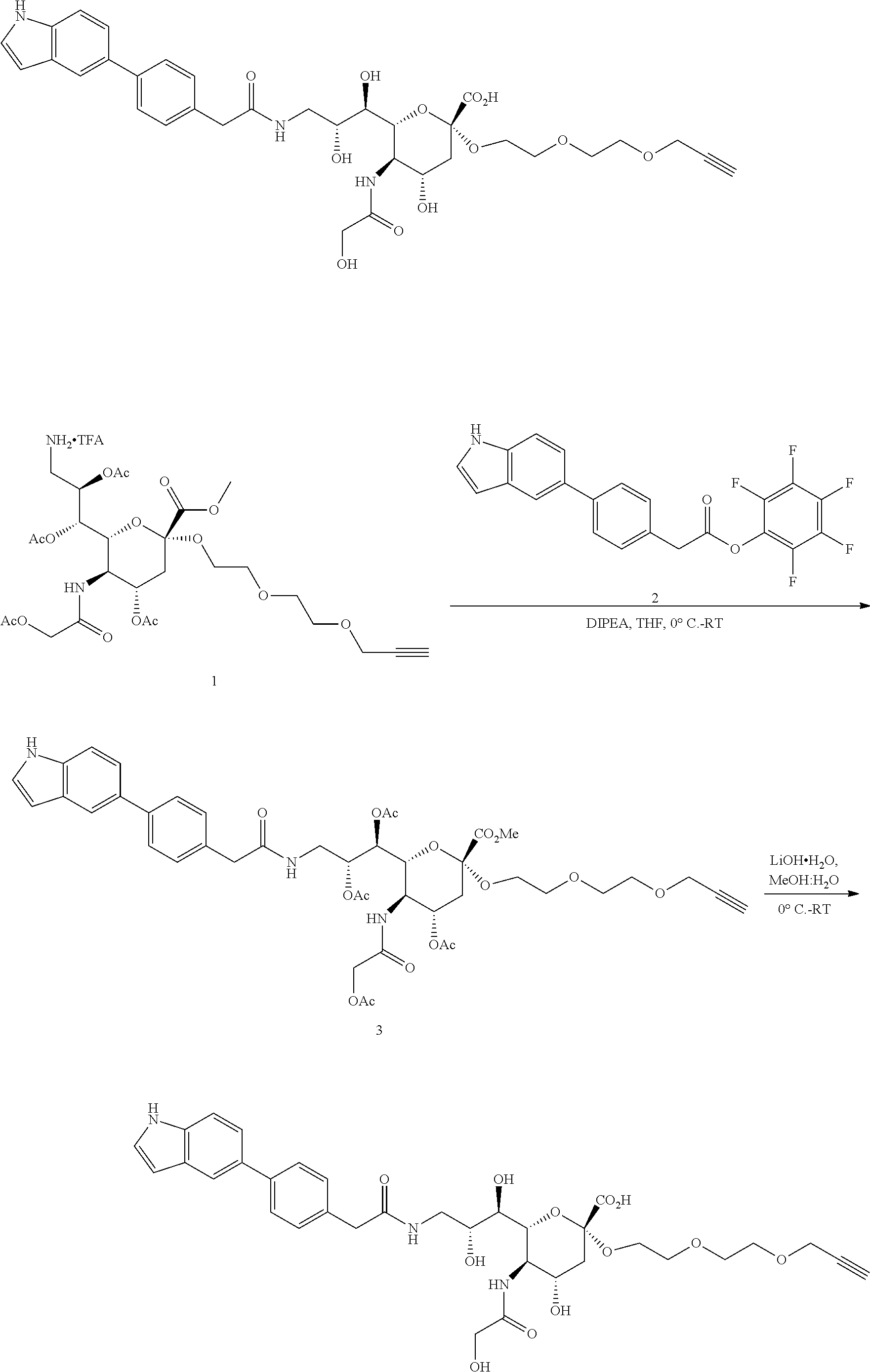

### Synthesis of (1R,2R)-3-(2-(4-(1H-indol-5-yl)phenyl)acetamido)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)propane-1,2-diyl diacetate (3)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-aminopropane-1,2-diyl diacetate (1, 0.075 g, 0.119 mmol) and perfluorophenyl 2-(4-(1H-indol-5-yl)phenyl)acetate (2, 0.099 g, 0.237 mmol) in tetrahydrofuran (3.0 mL), was added ethylbis(propan-2-yl)amine (0.103 mL, 0.593 mmol) at 0° C. then stirred at room temperature for 12 h. After completion the mixture was diluted with water and ethyl acetate. The organic layer was separated, dried, filtered, concentrated, and the crude residue obtained was purified by silica gel column chromatography and desired compound eluted with ethyl acetate in hexane as eluents to afford the desired product (1R,2R)-3-(2-(4-(1H-indol-5-yl)phenyl)acetamido)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)propane-1,2-diyl diacetate (3). Yield: 0.080 g, 77.93%; LCMS (ESI) m/z 864.0 [M−H]⁻.

### Synthesis of (2R,4S,5R,6R)-6-((1R,2R)-3-(2-(4-(1H-indol-5-yl)phenyl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 4)

To a stirred solution of (1R,2R)-3-(2-(4-(1H-indol-5-yl)phenyl)acetamido)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)propane-1,2-diyl diacetate (3, 0.080 g, 0.092 mmol) in methanol (2.0 mL), was added a solution of lithium hydroxide monohydrate (0.013 g, 0.554 mmol) in water (0.2 mL). The reaction mixture was stirred at room temperature for 6 h. After completion, the reaction mixture was treated with acidic resin (Dowex 50H⁺) up to pH ˜6 and the suspension was filtered, and the celite bed washed with methanol. The filtrate was concentrated, and the crude residue obtained was purified by prep HPLC using acetonitrile in water (+0.1% TFA) as eluents to afford the desired product to afford the desired product (2R,4S,5R,6R)-6-((1R,2R)-3-(2-(4-(1H-indol-5-yl)phenyl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 4). Yield: 0.006 g, 10%; LCMS m/z 684.4 [M+H]+; 1H NMR (400 MHz, methanol-d4) 7.77 (s, 1 h), 7.60 (d, J=8.4 Hz, 2H), 7.44 (d, J=8.4 Hz, 1H), 7.38-7.34 (m, 3H), 7.25 (d, J=3.2 Hz, 1H), 6.49 (d, J=3.2 Hz, 1H), 4.14 (d, J=2.4 Hz, 2H), 4.01 (s, 2H), 3.90-3.55 (m, 15H), 3.39-3.37 (m, 1H), 3.27-3.21 (m, 1H), 2.82 (t, J=2.4 Hz, 1H), 2.74-2.70 (dd, J=11.6 & 3.6 Hz, 1H), 1.77 (t, J=11.2 Hz, 1H).

### (2R,4S,5R,6R)-6-((1R,2R)-1,2-dihydroxy-3-(2-(4-phenyl-1H-1,2,3-triazol-1-yl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 177)

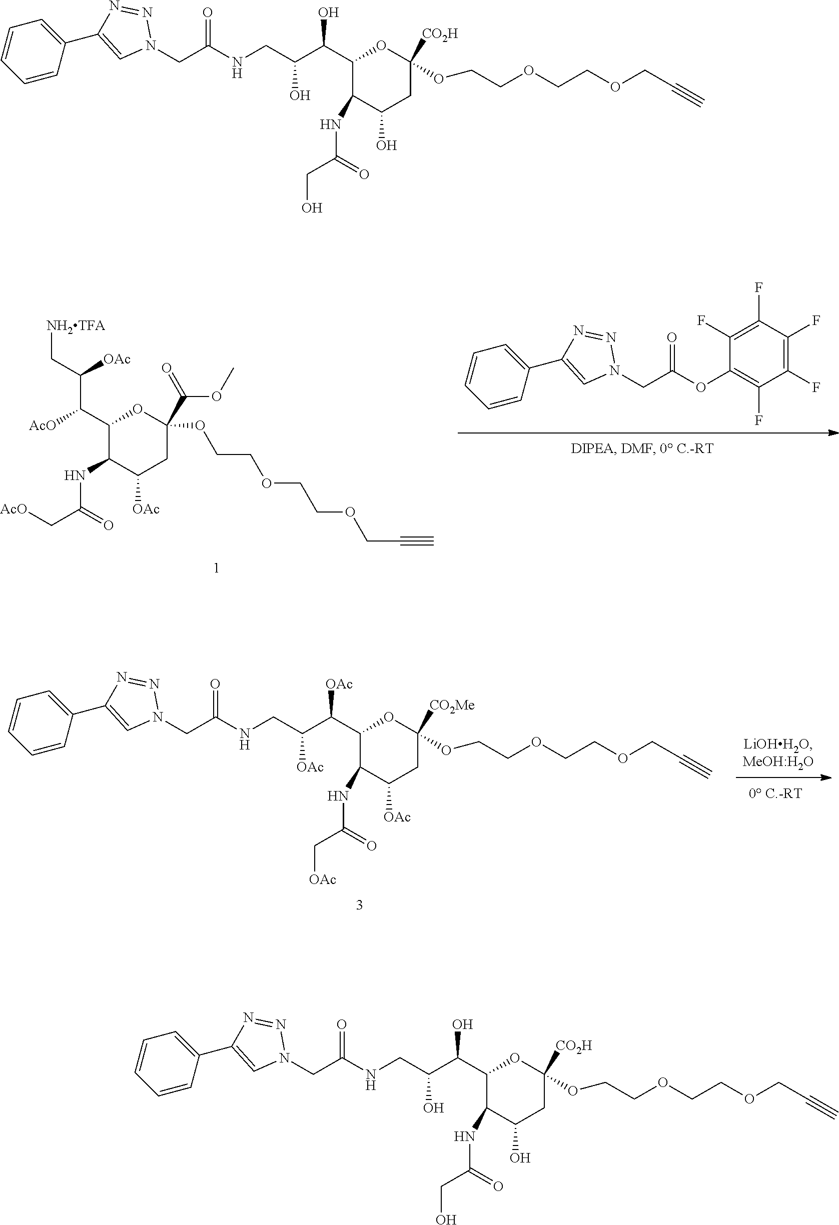

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(4-phenyl-1H-1,2,3-triazol-1-yl)acetamido)propane-1,2-diyl diacetate (3)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-((2,2,2-trifluoroacetyl)-14-azaneyl)propane-1,2-diyl diacetate (1, 0.30 g, 0.412 mmol) and perfluorophenyl 2-(4-phenyl-1H-1,2,3-triazol-1-yl)acetate (2, 0.304 g, 0.823 mmol) in tetrahydrofuran (10.0 mL), was added ethylbis(propan-2-yl)amine (0.213 mL, 1.23 mmol) at 0° C. then stirred at room temperature for 2 h. After completion, the reaction mixture was diluted with water and ethyl acetate. The organic layer was separated, dried, filtered, concentrated, and the crude residue obtained was purified by silica gel column chromatography and desired compound eluted with ethyl acetate in hexane as eluents to afford the desired product (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(4-phenyl-1H-1,2,3-triazol-1-yl)acetamido)propane-1,2-diyl diacetate (3). Yield: 0.180 g, 55%; LCMS (ESI) m/z 815.80 [M−H]⁻.

### Synthesis of (2R,4S,5R,6R)-6-((1R,2R)-1,2-dihydroxy-3-(2-(4-phenyl-1H-1,2,3-triazol-1-yl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 177)

To a stirred solution of methyl (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-(2-(4-phenyl-1H-1,2,3-triazol-1-yl)acetamido)propane-1,2-diyl diacetate (3, 0.180 g, 0.220 mmol) in methanol (5.0 mL), was added a solution of lithium hydroxide monohydrate (0.036 g, 1.68 mmol) in water (0.5 mL). The reaction mixture was stirred at room temperature for 6 h. After completion, the reaction mixture was treated with acidic resin (Dowex 50H⁺) up to pH ˜6 and the suspension was filtered. The filtrate was concentrated, and the crude residue obtained was purified by prep HPLC using acetonitrile in water (+0.1% TFA) as eluents to afford the desired product to afford the desired product (2R,4S,5R,6R)-6-((1R,2R)-1,2-dihydroxy-3-(2-(4-phenyl-1H-1,2,3-triazol-1-yl)acetamido)propyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 177). Yield: 0.009 g, 11%; LCMS m/z 636.24 [M+H]+; ¹H NMR (400 MHz, methanol-d₄) δ 8.35 (s, 1H), 8.31 (m, 1H), 7.91 (d, J=7.2 Hz), 7.84 (d, J=8.0 Hz, 2H), 7.44 (t, J=7.6 Hz, 2H), 7.37-7.33 (m, 1H), 5.22 (s, 2H), 4.18 (d, J=2.4 Hz, 2H), 4.04 (s, 2H), 3.94-3.62 (m, 13H), 3.48-3.38 (m, 2H), 2.84-2.83 (t, J=4.4 Hz, 2H), 2.75-2.71 (dd, J=3.2 Hz, 12.4 Hz, 1H), 1.46 (t, J=12.0 Hz, 1H).

### (2R,4S,5R,6R)-5-(2-cyanoacetamido)-6-((1R,2R)-1,2-dihydroxy-3-(2-(4′-hydroxy-[1,1′-biphenyl]-4-yl)acetamido)propyl)-4-hydroxy-2-(2-(2-(prop-2-yn-1-yloxy)ethoxy)ethoxy)tetrahydro-2H-pyran-2-carboxylic acid (Compound 58)

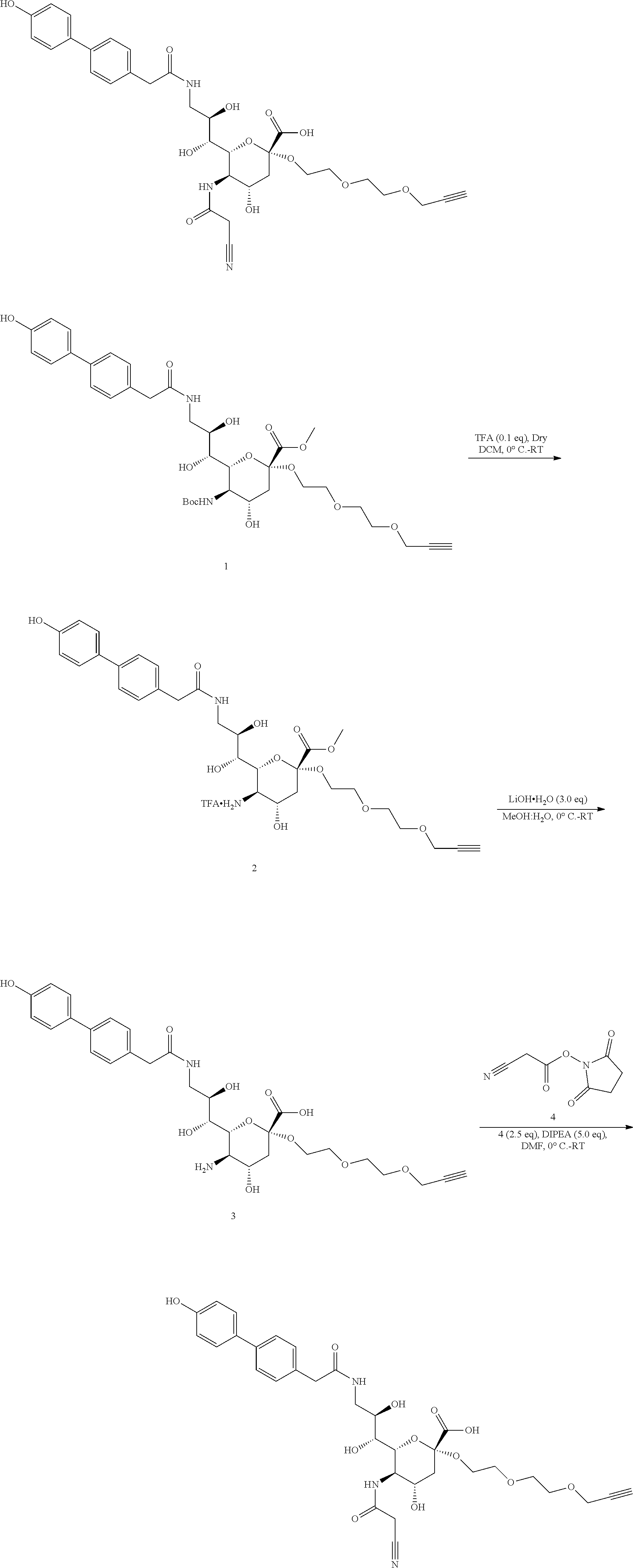

### Synthesis of methyl (2R,4S,5R,6R)-6-((1R,2R)-1,2-dihydroxy-3-(2-(4′-hydroxy-[1,1′-biphenyl]-4-yl)acetamido)propyl)-4-hydroxy-2-(2-(2-(prop-2-yn-1-yloxy)ethoxy)ethoxy)-5-((2,2,2-trifluoroacetyl)-14-azaneyl)tetrahydro-2H-pyran-2-carboxylate (3)

To a solution of methyl (2R,4S,5R,6R)-5-((tert-butoxycarbonyl)amino)-6-((1R,2R)-1,2-dihydroxy-3-(2-(4′-hydroxy-[1,1′-biphenyl]-4-yl)acetamido)propyl)-4-hydroxy-2-(2-(2-(prop-2-yn-1-yloxy)ethoxy)ethoxy)tetrahydro-2H-pyran-2-carboxylate (1, 0.20 g, 0.280 mmol), in anhydrous dichloromethane (2.0 mL), trifluoroacetic acid (0.5 mL) was added at 0° C., was stirred at room temperature for 4 h. After completion, solvent was concentrated, and dried to afford the desired product methyl (2R,4S,5R,6R)-6-((1R,2R)-1,2-dihydroxy-3-(2-(4′-hydroxy-[1,1′-biphenyl]-4-yl)acetamido)propyl)-4-hydroxy-2-(2-(2-(prop-2-yn-1-yloxy)ethoxy)ethoxy)-5-((2,2,2-trifluoroacetyl)-14-azaneyl)tetrahydro-2H-pyran-2-carboxylate (2). The crude residue obtained was used for the next step without further purification. Yield: 0.0170 g, Crude; LCMS (ESI) m/z 617.05 [M+H]⁺.

### Synthesis of (2R,4S,5R,6R)-5-amino-6-((1R,2R)-1,2-dihydroxy-3-(2-(4′-hydroxy-[1,1′-biphenyl]-4-yl)acetamido)propyl)-4-hydroxy-2-(2-(2-(prop-2-yn-1-yloxy)ethoxy)ethoxy)tetrahydro-2H-pyran-2-carboxylic acid (3)

To a stirred solution of methyl (2R,4S,5R,6R)-5-amino-6-((1R,2R)-1,2-dihydroxy-3-(2-(4′-hydroxy-[1,1′-biphenyl]-4-yl)acetamido)propyl)-4-hydroxy-2-(2-(2-(prop-2-yn-1-yloxy)ethoxy)ethoxy)tetrahydro-2H-pyran-2-carboxylate (2, 0.170 g, 0. mmol) in methanol (5 mL), was added a solution of lithium hydroxide monohydrate (0.0 g, 0.275 mmol) in water (1.0 mL). The reaction mixture was stirred at room temperature for 6 h. After completion, the reaction mixture was treated with acidic resin (Dowex 50, H⁺) to PH ˜6 and the suspension was filtered. The filtrate was concentrated. to afford the desired product (2R,4S,5R,6R)-5-amino-6-((1R,2R)-1,2-dihydroxy-3-(2-(4′-hydroxy-[1,1′-biphenyl]-4-yl)acetamido)propyl)-4-hydroxy-2-(2-(2-(prop-2-yn-1-yloxy)ethoxy)ethoxy)tetrahydro-2H-pyran-2-carboxylic acid (3). The crude residue obtained was used for the next step without further purification. Yield: 0.0150 g, Crude; LCMS (ESI) m/z 603.0[M+H]⁺.

### Synthesis of (2R,4S,5R,6R)-5-(2-cyanoacetamido)-6-((1R,2R)-1,2-dihydroxy-3-(2-(4′-hydroxy-[1,1′-biphenyl]-4-yl)acetamido)propyl)-4-hydroxy-2-(2-(2-(prop-2-yn-1-yloxy)ethoxy)ethoxy)tetrahydro-2H-pyran-2-carboxylic acid (Cpd. 58)

To a solution of (2R,4S,5R,6R)-5-amino-6-((1R,2R)-1,2-dihydroxy-3-(2-(4′-hydroxy-[1,1′-biphenyl]-4-yl)acetamido)propyl)-4-hydroxy-2-(2-(2-(prop-2-yn-1-yloxy)ethoxy)ethoxy)tetrahydro-2H-pyran-2-carboxylic acid (3, 0.150 g, 0.260 mmol) in DMF (4.0 mL) was added diisopropyl ethylamine (0.150 mL, 0.850 mmol) followed by 2,5-dioxopyrrolidin-1-yl 2-cyanoacetate (4, 0.032 g, 0.33 mmol) slowly at 0° C. The reaction was stirred at 0° C. After completion, the mixture was concentrated, and the crude residue obtained was purified by prep HPLC using acetonitrile in water (+0.1% TFA) as eluents to afford the desired product to afford the desired product.

A: (2R,4S,5R,6R)-5-(2-cyanoacetamido)-6-((1R,2R)-1,2-dihydroxy-3-(2-(4′-hydroxy-[1,1′-biphenyl]-4-yl)acetamido)propyl)-4-hydroxy-2-(2-(2-(prop-2-yn-1-yloxy)ethoxy)ethoxy)tetrahydro-2H-pyran-2-carboxylic acid (Cpd. 58). Yield: 0.031 g, 18%; LCMS m/z 670.40 [M+H]+; ¹H NMR (400 MHz, methanol-d₄) 7.51 (d, J=8.4 Hz, 2H), 7.45-7.43 (m, 2H), 7.34 (d, J=8.4 Hz, 2H), 6.85-6.83 (m, 2H), 4.17 (d, J=2.4 Hz, 2H), 3.90-3.85 (m, 2H), 3.83-3.51 (m, 13H), 3.51 (d, J=1.2 Hz, 2H), 3.38-3.35 (m, 1H), 3.30-3.25 (m, 1H), 2.82 (t, J=2.4 Hz, 1H), 2.71-2.66 (dd, J=12.8 Hz & 4.4 Hz, 1H), 1.74 (t, J=12.4 Hz, 1H),

### (2R,4S,5R,6R)-5-(2-acetamidoacetamido)-6-((1R,2R)-1,2-dihydroxy-3-(2-(4′-hydroxy-[1,1′-biphenyl]-4-yl)acetamido)propyl)-4-hydroxy-2-(2-(2-(prop-2-yn-1-yloxy)ethoxy)ethoxy)tetrahydro-2H-pyran-2-carboxylic acid (Compound 57)

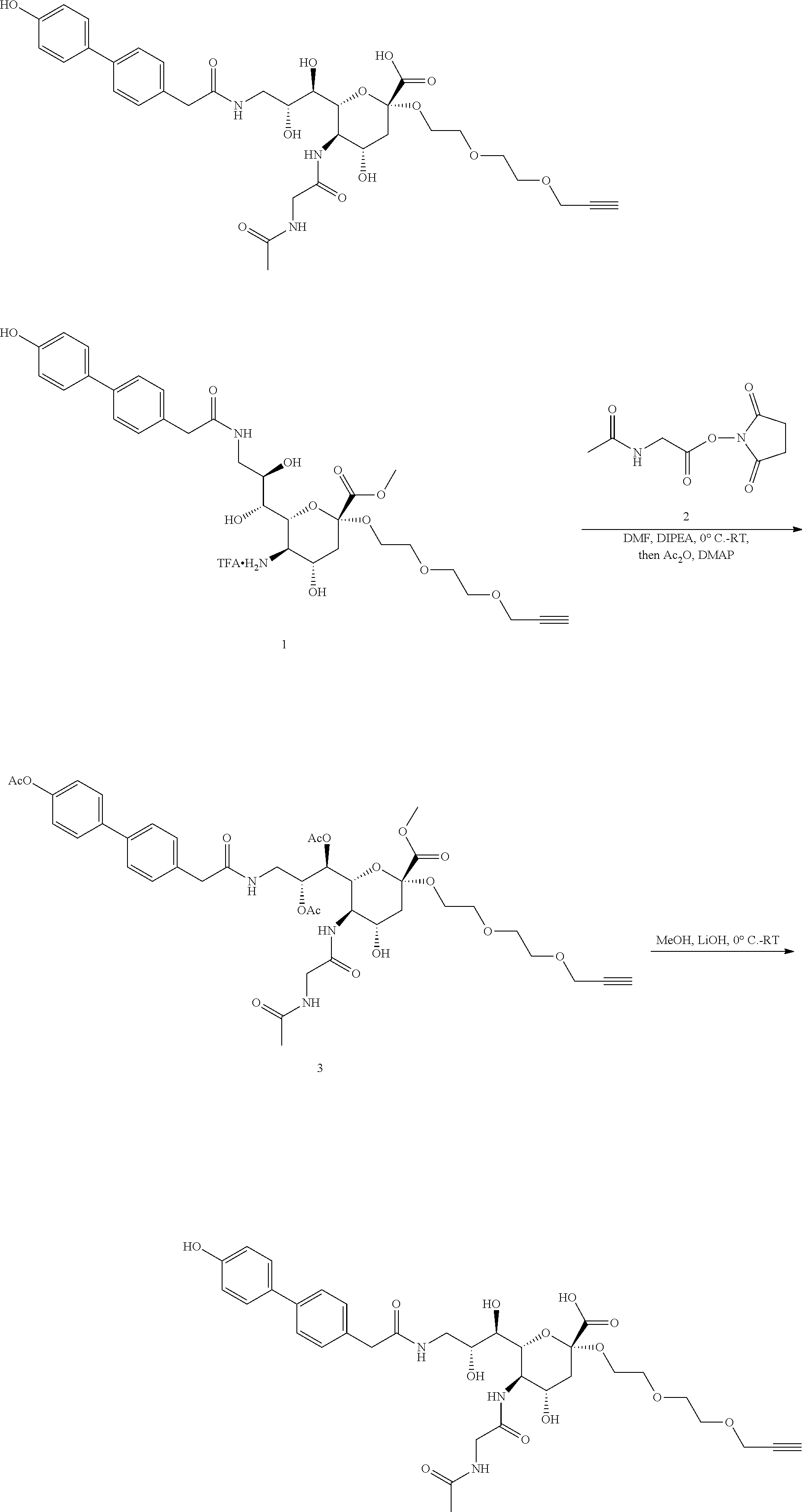

### Synthesis of (1R,2R)-1-((2R,3R,4S,6R)-3-(2-acetamidoacetamido)-4-acetoxy-6-(methoxycarbonyl)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (3)

To a stirred solution of methyl (2R,4S,5R,6R)-6-((1R,2R)-1,2-dihydroxy-3-(2-(4′-hydroxy-[1,1′-biphenyl]-4-yl)acetamido)propyl)-4-hydroxy-2-(2-(2-(prop-2-yn-1-yloxy)ethoxy)ethoxy)-5-((2,2,2-trifluoroacetyl)-14-azaneyl)tetrahydro-2H-pyran-2-carboxylate (1, 0.20 g, 0.33 mmol) in DMF (4.0 mL) was added diisopropyl ethylamine (0.28 mL, 1.62 mmol) followed by 2,5-dioxopyrrolidin-1-yl acetylglycinate (2, 0.104 g, 0.33 mmol) slowly at 0° C. The reaction mixture was stirred at 0° C. After completion, acetic anhydride (0.53 mL, 3.3 mmol) and 4-dimethylaminopyrdine (0.1 g, 0.33 mmol) was added and stirred for 12 h. The reaction mixture was concentrated. The crude was diluted with ethyl acetate (50 mL), washed once with water, and twice with brine. The organics were dried, filtered, and filtrate was concentrated, and the crude residue obtained was purified by silica gel column chromatography (30-70% ethyl acetate in heptane) to afford the desired product (1R,2R)-1-((2R,3R,4S,6R)-3-(2-acetamidoacetamido)-4-acetoxy-6-(methoxycarbonyl)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (3). Yield: 0.11 g, 39%; LCMS (ESI) m/z 883.95 [M+H]⁺.

### Synthesis of (2R,4S,5R,6R)-5-(2-acetamidoacetamido)-6-((1R,2R)-1,2-dihydroxy-3-(2-(4′-hydroxy-[1,1′-biphenyl]-4-yl)acetamido)propyl)-4-hydroxy-2-(2-(2-(prop-2-yn-1-yloxy)ethoxy)ethoxy)tetrahydro-2H-pyran-2-carboxylic acid (Cpd. 57)

To a stirred solution of (1R,2R)-1-((2R,3R,4S,6R)-3-(2-acetamidoacetamido)-4-acetoxy-6-(methoxycarbonyl)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (3, 0.20 g, 0.24 mmol) in methanol (5 mL), was added a solution of lithium hydroxide monohydrate (0.034 g, 1.44 mmol) in water (1.0 mL). The reaction mixture was stirred at room temperature for 6 h. After completion, the reaction mixture was treated with acidic resin (Dowex 50, H⁺) to PH ˜6 and the suspension was filtered After completion, the mixture was concentrated, and the crude residue obtained was purified by prep HPLC using acetonitrile in water (+0.1% TFA) as eluents to afford the desired product to afford the desired product.

A: (2R,4S,5R,6R)-5-(2-acetamidoacetamido)-6-((1R,2R)-1,2-dihydroxy-3-(2-(4′-hydroxy-[1,1′-biphenyl]-4-yl)acetamido)propyl)-4-hydroxy-2-(2-(2-(prop-2-yn-1-yloxy)ethoxy)ethoxy)tetrahydro-2H-pyran-2-carboxylic acid (Cpd. 57). Yield: 0.042 g, 26%; LCMS m/z 702.35 [M+H]+; ¹H NMR (400 MHz, methanol-d₄) δ 8.0 (d, J=8.4 Hz, 1H), 7.50 (d, J=8.0 Hz, 2H) 7.46-7.42 (m, 2H), 7.34 (d, J=8.4 Hz, 2H), 6.86-6.82 (m, 2H), 4.17 (d, J=2.4 Hz, 2H), 3.89-3.70 (m, 6H), 3.68-3.56 (m, 11H), 3.43 (dd, J=8.8 Hz & 1.6 Hz, 1H), 3.30-3.22 (m, 1H), 2.83 (t, J=2.4 Hz, 1H), 2.70 (dd, J=12.8 Hz & 4.4 Hz, 1H), 1.98 (s, 3H), 1.73 (t, J=12.0 Hz, 1H).

### (2R,4S,5R,6R)-6-((1R,2R)-1,2-dihydroxy-3-palmitamidopropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Compound 13)

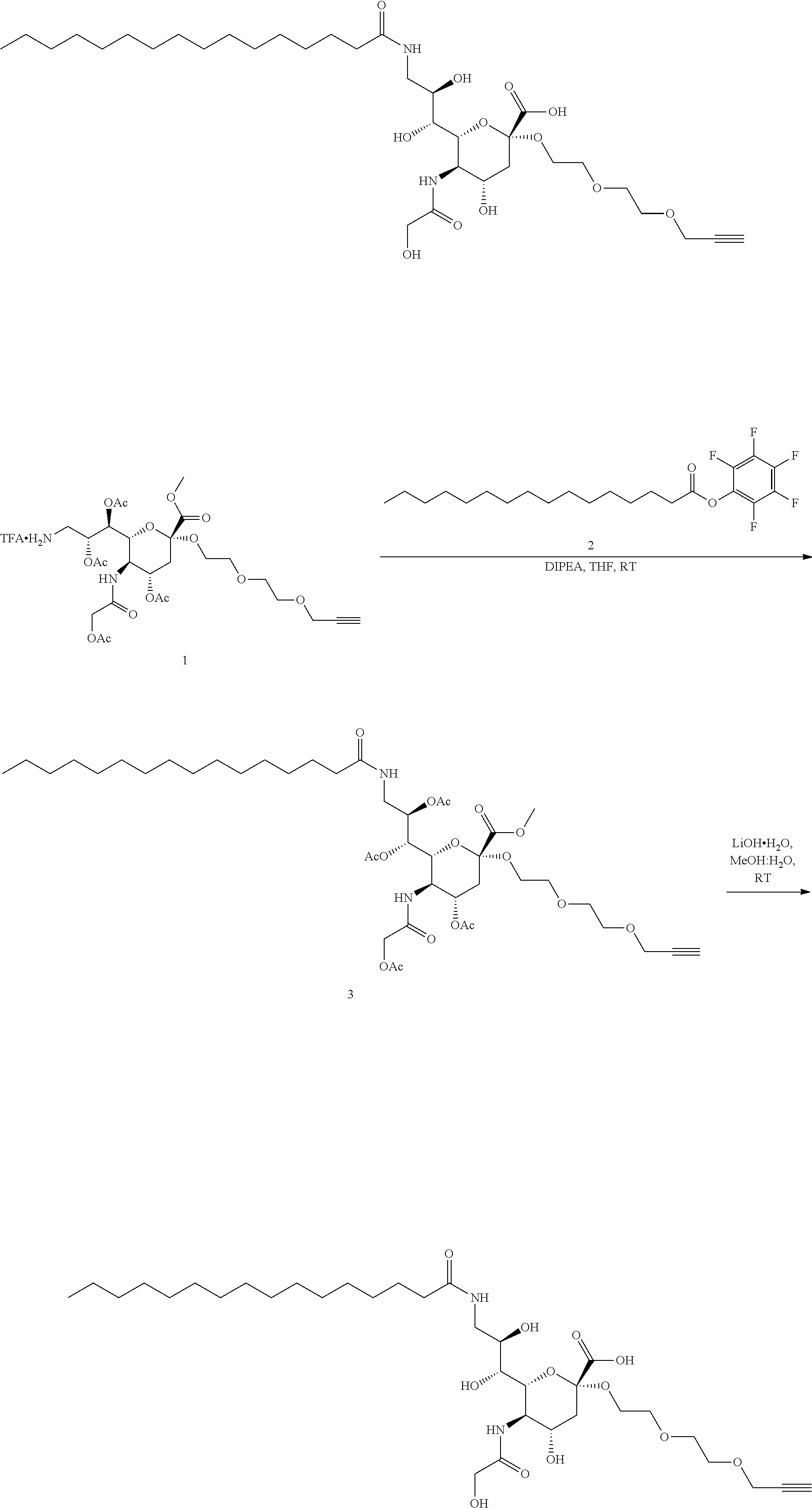

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-palmitamidopropane-1,2-diyl diacetate (3)

To a solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-aminopropane-1,2-diyl diacetate (1, 0.50 g, 0.790 mmol), in tetrahydrofuran (5.0 mL), ware added ethylbis(propan-2-yl)amine (0.68 mL, 0.689 mmol) and perfluorophenyl palmitate (2, 0.267 g, 0.632 mmol) at 0° C. then stirred at room temperature for 12 h. After completion the mixture was concentrated, and the crude residue obtained was purified by silica gel column chromatography using ethyl acetate in hexane as eluents to afford the desired product (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-palmitamidopropane-1,2-diyl diacetate (3). Yield: 0.35 g, 51%; ELSD-MS 871.25 [M+H]⁺.

### Synthesis of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-palmitamidopropane-1,2-diyl diacetate (Cpd. No. Cpd. 13)

To a stirred solution of (1R,2R)-1-((2R,3R,4S,6R)-4-acetoxy-3-(2-acetoxyacetamido)-6-(methoxycarbonyl)-6-(2-(2-(prop-2-yn-1-yloxy)ethoxy)ethoxy)tetrahydro-2H-pyran-2-yl)-3-palmitamidopropane-1,2-diyl diacetate (3, 0.35 g, 0.402 mmol) in methanol (4.0 mL), was added a solution of lithium hydroxide monohydrate (0.101 g, 2.410 mmol) in water (0.40 mL). The reaction mixture was stirred at room temperature for 6 h. After completion, the reaction mixture was treated with acidic resin (Dowex 50H+) up to pH ˜6 and the suspension was filtered. The filtrate was concentrated, and the crude residue obtained was purified by prep HPLC using acetonitrile in water (+0.1% TFA) as eluents to afford the desired product (2R,4S,5R,6R)-6-((1R,2R)-1,2-dihydroxy-3-palmitamidopropyl)-4-hydroxy-5-(2-hydroxyacetamido)-2-(2-(2-(prop-2-yn-1-yloxy)ethoxy)ethoxy)tetrahydro-2H-pyran-2-carboxylic acid (Cpd. 13). Yield: 0.030 g, 11%; ELSD-MS 689.25 [M+H]+; ¹H NMR (400 MHz, methanol-d₄) δ 4.19 (d, J=2.4 Hz, 2H), 4.02 (s, 2H), 3.93-3.81 (m, 4H), 3.74-3.72 (m, 1H), 3.69-3.59 (m, 8H), 3.36 (dd, J=9.2 & 1.6 Hz, 1H), 3.30-3.20 (m, 1H), 2.83 (t, J=2.4 Hz, 1H), 2.73 (dd, J=12.4 & 3.6 Hz, 1H), 2.21 (t, J=7.2 Hz, 2H), 1.75 (t, J=12.8 Hz, 1H), 1.60 (t, J=6.8 Hz, 2H), 1.30-1.28 (m, 24H), 0.90 (t, J=6.4 Hz, 3H).

### (2R,4S,5R,6R)-2-(3-carboxypropoxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Compound 118)

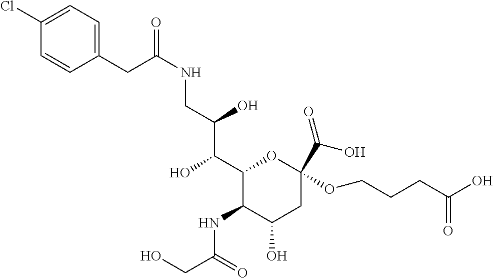 [SMILES: O=C(O)CCCO[C@]1(C(=O)O)C[C@H](O)[C@@H](NC(=O)CO)[C@H]([C@H](O)[C@H](O)CNC(=O)Cc2ccc(Cl)cc2)O1]

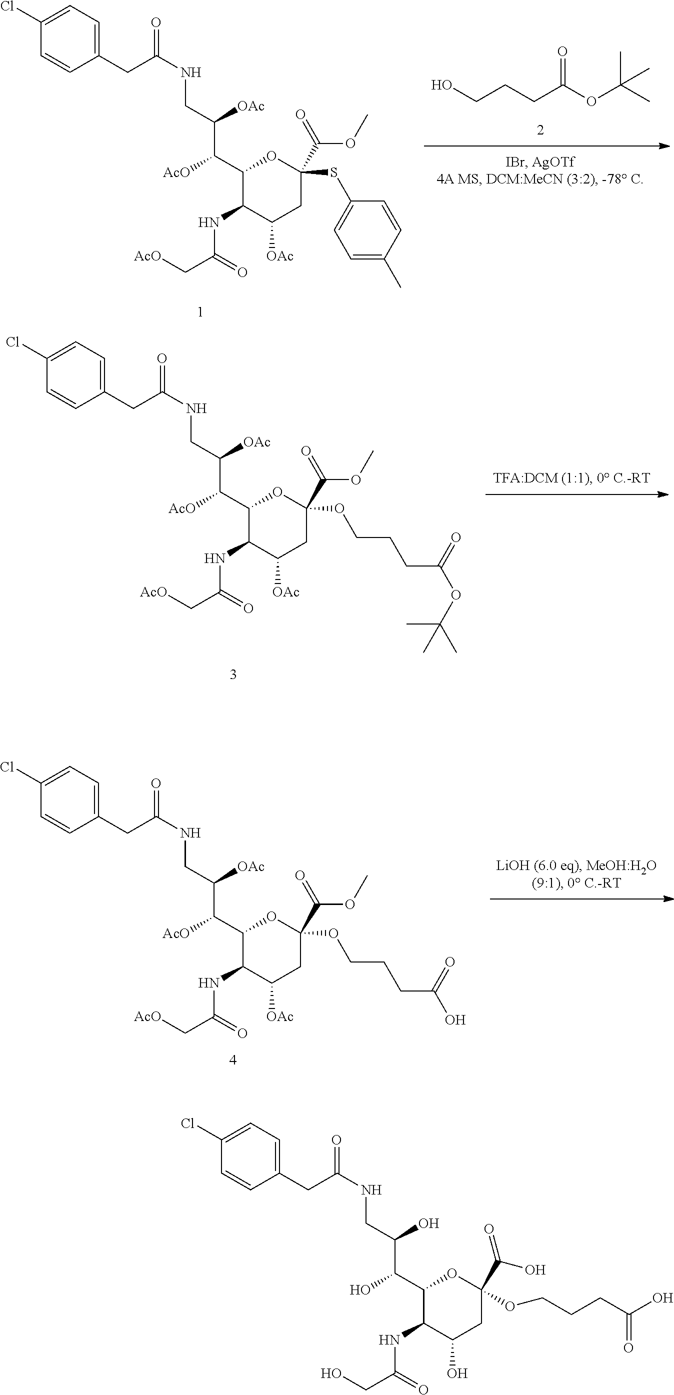 [SMILES: COC(=O)[C@]1(Sc2ccc(C)cc2)C[C@H](OC(C)=O)[C@@H](NC(=O)COC(C)=O)[C@H]([C@H](OC(C)=O)[C@@H](CNC(=O)Cc2ccc(Cl)cc2)OC(C)=O)O1 ; CC(C)(C)OC(=O)CCCO ; COC(=O)[C@@]1(OCCCC(=O)OC(C)(C)C)C[C@H](OC(C)=O)[C@@H](NC(=O)COC(C)=O)[C@H]([C@H](OC(C)=O)[C@@H](CNC(=O)Cc2ccc(Cl)cc2)OC(C)=O)O1 ; COC(=O)[C@@]1(OCCCC(=O)O)C[C@H](OC(C)=O)[C@@H](NC(=O)COC(C)=O)[C@H]([C@H](OC(C)=O)[C@@H](CNC(=O)Cc2ccc(Cl)cc2)OC(C)=O)O1 ; O=C(O)CCCO[C@]1(C(=O)O)C[C@H](O)[C@@H](NC(=O)CO)[C@H]([C@H](O)[C@H](O)CNC(=O)Cc2ccc(Cl)cc2)O1]

### Synthesis of (1R,2R)-1-((2R,3R,4S,6R)-4-acetoxy-3-(2-acetoxyacetamido)-6-(4-(tert-butoxy)-4-oxobutoxy)-6-(methoxycarbonyl)tetrahydro-2H-pyran-2-yl)-3-(2-(4-chlorophenyl)acetamido)propane-1,2-diyl diacetate (3)

A solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (1, 0.15 g, 0.19 mmol) and tert-butyl 4-hydroxybutanoate (2, 0.16 g, 0.98 mmol), silver(1+) trifluoromethanesulfonate (0.10 g, 0.39 mmol) and activated 4 Å powdered molecular sieves (1.00 g) in anhydrous dichloromethane (35 mL) and anhydrous acetonitrile (60 mL) was stirred at room temperature for 1 h. The solution was cooled to −78° C. and a solution of iodobromane (0.06 g, 0.29 mmol) in dichloromethane (5.0 mL) was added dropwise. The reaction mixture was stirred at the same temperature for 2 h. After completion, the reaction mixture was quenched by addition of triethylamine (1.0 mL) and warmed up to room temperature. The reaction mixture was filtered and. The filtrate was washed with a a saturated solution of sodium bicarbonate and dried, filtered, and concentrated. The crude residue obtained was purified by silica gel column chromatography using ethyl acetate in heptane as eluents to afford the desired product (1R,2R)-1-((2R,3R,4S,6R)-4-acetoxy-3-(2-acetoxyacetamido)-6-(4-(tert-butoxy)-4-oxobutoxy)-6-(methoxycarbonyl)tetrahydro-2H-pyran-2-yl)-3-(2-(4-chlorophenyl)acetamido)propane-1,2-diyl diacetate (3). Yield: 0.11 g, 70%; LCMS m/z 800.10 [M−H]⁻.

### Synthesis of 4-(((2R,4S,5R,6R)-4-acetoxy-5-(2-acetoxyacetamido)-6-((1R,2R)-1,2-diacetoxy-3-(2-(4-chlorophenyl)acetamido)propyl)-2-(methoxycarbonyl)tetrahydro-2H-pyran-2-yl)oxy)butanoic acid (4)

To a stirred solution of (1R,2R)-1-((2R,3R,4S,6R)-4-acetoxy-3-(2-acetoxyacetamido)-6-(4-(tert-butoxy)-4-oxobutoxy)-6-(methoxycarbonyl)tetrahydro-2H-pyran-2-yl)-3-(2-(4-chlorophenyl)acetamido)propane-1,2-diyl diacetate (3, 0.12 g, 0.15 mmol), in anhydrous dichloromethane (3.0 mL), trifluoroacetic acid (1.0 mL) was added at 0° C. then stirred at room temperature for 3 h. After completion the solvent was concentrated to afford the desired product 4-(((2R,4S,5R,6R)-4-acetoxy-5-(2-acetoxyacetamido)-6-((1R,2R)-1,2-diacetoxy-3-(2-(4-chlorophenyl)acetamido)propyl)-2-(methoxycarbonyl)tetrahydro-2H-pyran-2-yl)oxy)butanoic acid (4). The crude was used for the next step without further purification. Yield: 0.10 g, Crude; LCMS (ESI) m/z 693.90 [M+H3O]⁺.

### Synthesis of (2R,4S,5R,6R)-2-(3-carboxypropoxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Cpd.118)

To a stirred solution of 4-(((2R,4S,5R,6R)-4-acetoxy-5-(2-acetoxyacetamido)-6-((1R,2R)-1,2-diacetoxy-3-(2-(4-chlorophenyl)acetamido)propyl)-2-(methoxycarbonyl)tetrahydro-2H-pyran-2-yl)oxy)butanoic acid (4, 0.10 g, 0.13 mmol) in methanol (5 mL), was added a solution of lithium hydroxide monohydrate (0.019 g, 0.81 mmol) in water (0.5 mL). The reaction mixture was stirred at room temperature for 6 h. After completion, the reaction mixture was treated with acidic resin (Dowex 50, H⁺) to pH˜6 and the suspension was filtered. The filtrate was concentrated, and the crude residue obtained was purified by prep HPLC using acetonitrile in water (+0.1% TFA) as eluents to afford the desired product ((2R,4S,5R,6R)-2-(3-carboxypropoxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Cpd. 118). Yield: 0.019 g, 22%; LCMS m/z 563.35 [M+H]+; ¹H NMR (400 MHz, Methanol-d₄) δ 7.34-7.29 (m, 4H), 4.03 (s, 2H), 3.93-3.79 (m, 4H), 3.75-3.73 (m, 1H), 3.65 (dd, J=14.0 Hz & 3.2 Hz, 1H), 3.55 (s, 2H), 3.51-3.45 (m, 1H), 3.38 (dd, J=8.8 Hz & 1.6 Hz, 1H), 3.32-3.23 (m, 1H), 2.74 (dd, J=12.4 Hz & 4.0 Hz, 1H), 2.44-2.35 (m, 2H), 1.88-1.82 (m, 2H), 1.75 (t, J=12.4 Hz, 1H).

### (2R,4S,5R,6R)-6-((1R,2R)-3-(2-(4-(1H-benzo[d]imidazol-5-yl)phenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Compound 3)

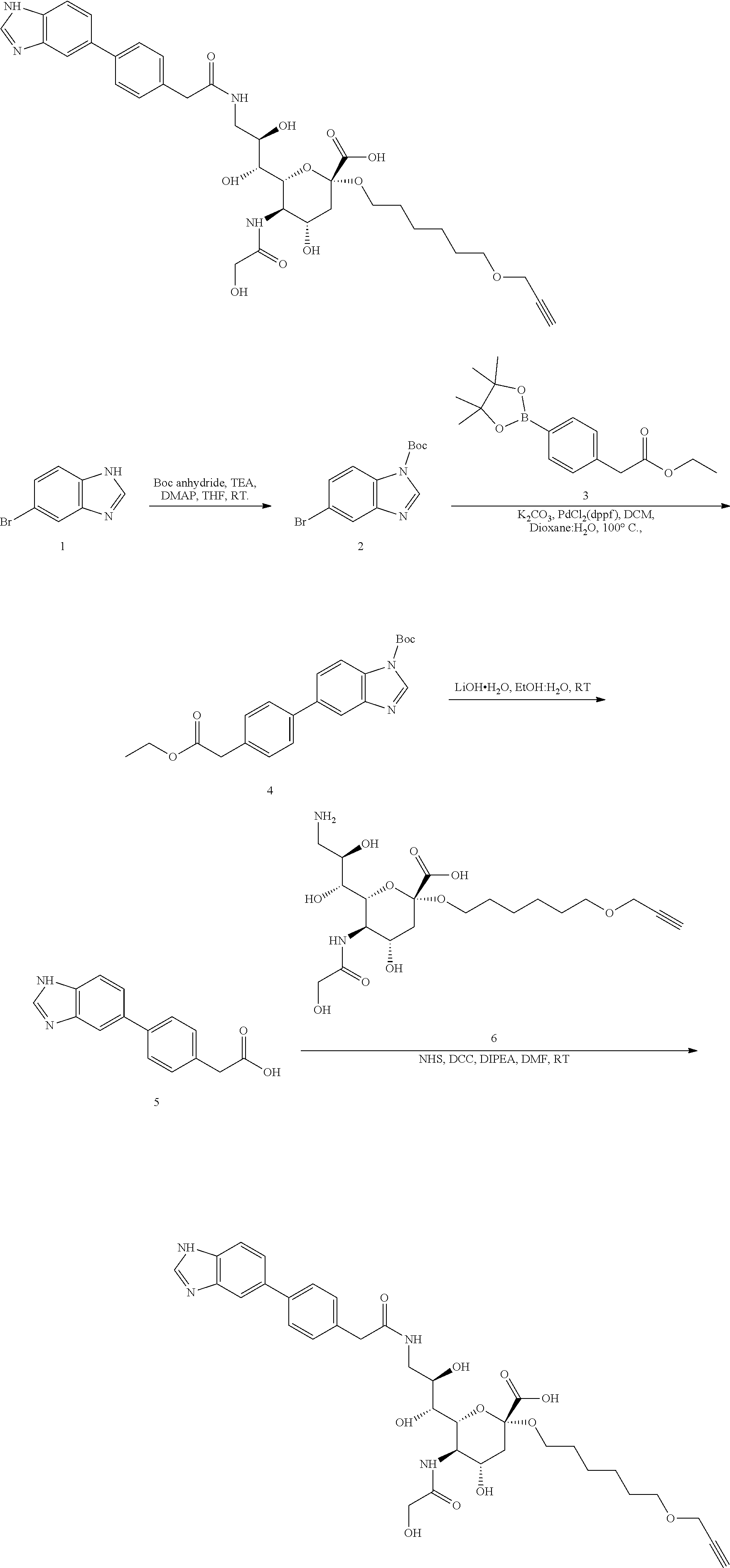

### Synthesis of tert-butyl 5-bromo-1H-benzo[d]imidazole-1-carboxylate (2)

To a stirred solution of 5-bromo-1H-benzo[d]imidazole (1, 2.0 g, 10.20 mmol) in tetrahydrofuran (20.0 mL) were successively added triethylamine (4.24 mL, 30.50 mmol), N,N-dimethyl-4-pyridylamine (0.620 g, 5.08 mmol) and tert-butoxy-tert-butoxycarbonylformylate (4.60 mL, 20.30 mmol) at 0° C. The reaction mixture was stirred at room temperature for 16 h. After completion, the reaction mixture was diluted with water and ethyl acetate. The organic layer was separated and dried, filtered, and concentrated. The crude compound was purified by silica gel column chromatography using ethyl acetate in heptane as eluents to afford the desired product tert-butyl 5-bromo-1H-benzo[d]imidazole-1-carboxylate (2) Yield: 1.25 g, 41.44%; LCMS m/z 296.95 [M+H]+.

### Synthesis of tert-butyl 5-(4-(2-ethoxy-2-oxoethyl)phenyl)-1H-benzo[d]imidazole-1-carboxylate (4)

Stirred solution of tert-butyl 5-bromo-1H-1,3-benzimidazole-1-carboxylate (2, 0.90 g, 3.03 mmol), ethyl 2-(4-(4,4,5,5-tetramethyl-1,3,2-dioxaborolan-2-yl)phenyl)acetate (3, 0.879 g, 3.03 mmol) and dipotassium carbonate (1.26 g, 9.09 mmol) in 1,4-dioxane (8.0 mL) and water (1.0 mL) was degassed by bubbling argon gas for 10 min. Pd(dppf)C₂·DCM (0.443 g, 0.606 mmol) was added at room temperature and the mixture was stirred for 6 h at 100° C. After completion, the reaction mixture was diluted with water and extracted with ethyl acetate. The organic layer was separated, dried, filtered, and concentrated. The crude residue obtained was purified by silica gel column chromatography using ethyl acetate in heptane as eluents to afford the desired product tert-butyl 5-(4-(2-ethoxy-2-oxoethyl)phenyl)-1H-benzo[d]imidazole-1-carboxylate (4). Yield: 0.550 g, 48%; LCMS m/z 381.10 [M+H]⁺.

### Synthesis of 2-(4-(1H-benzo[d]imidazol-5-yl)phenyl)acetic acid (5)

To a stirred solution of tert-butyl 5-(4-(2-ethoxy-2-oxoethyl)phenyl)-1H-benzo[d]imidazole-1-carboxylate (4, 0.20 g, 0.526 mmol) in Ethanol (3.0 mL) was added a solution of lithium hydroxide monohydrate (0.066 g, 1.58 mmol) in water (0.50 ml). The reaction mixture was stirred at room temperature for 6 h. After completion, the reaction mixture was treated with 1N HCl solution and extracted with ethyl acetate. The combined organic layer was concentrated to afford the desired product 2-(4-(1H-benzo[d]imidazol-5-yl)phenyl)acetic acid (5) as a crude. Yield: 0.130 g, 98%; LCMS m/z 253.10 [M+H]⁺.

### Synthesis of (2R,4S,5R,6R)-6-((1R,2R)-3-(2-(4-(1H-benzo[d]imidazol-5-yl)phenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Cpd. 3)

To a stirred solution of 2-(4-(1H-benzo[d]imidazol-5-yl)phenyl)acetic acid (5, 0.150 g, 0.594 mmol) in N,N-dimethylformamide (3.0 mL), N,N′-dicyclohexylcarbodiimide (0.122 g, 0.594 mmol) and N-hydroxysuccinimide (0.068 g, 0.594 mmol) was added at 0° C. The reaction mixture was stirred at room temperature for 3 h. (2R,4S,5R,6R)-6-((1R,2R)-3-amino-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (0.274 g, 0.594 mmol) was added at 0° C. then stirred at room temperature for 4 h. After completion, the reaction mixture was concentrated, and the crude residue obtained was purified by prep HPLC using acetonitrile in water as eluents (+0.05% TFA) to afford the desired product (2R,4S,5R,6R)-6-((1R,2R)-3-(2-(4-(1H-benzo[d]imidazol-5-yl)phenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Cpd. 3). Yield: 0.008 g, 2%; LCMS (ESI) m/z 697.45 [M+H]+; ¹H NMR (400 MHz, MeOD-d4): δ 8.89 (s, 1H), 7.94 (s, 1H), 7.81-7.75 (m, 2H), 7.64 (d, J=8.4 Hz, 2H), 7.43 (d, J=8.0 Hz, 2H), 4.12-4.08 (m, 4H), 4.03-4.01 (m, 3H), 3.88-3.82 (m, 2H), 3.81-3.74 (m, 3H), 3.72-3.67 (m, 4H), 3.61 (s, 2H), 3.51 (t, J=6.4 Hz, 2H), 3.45 (t, J=6.8 Hz, 2H), 3.38-3.36 (m, 1H), 3.26-3.21 (m, 1H), 2.82-2.80 (m, 1H), 2.74 (dd, J=12.4 Hz & 4.0 Hz, 1H), 2.32 (s, 1H), 1.70-1.67 (m, 1H), 1.57-1.50 (m, 2H), 1.38-1.37 (m, 2H), 1.30-1.29 (m, 4H).

### (2R,4S,5R,6R)-6-((1R,2R)-3-(2-(4-(1H-indazol-5-yl)phenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Compound 2)

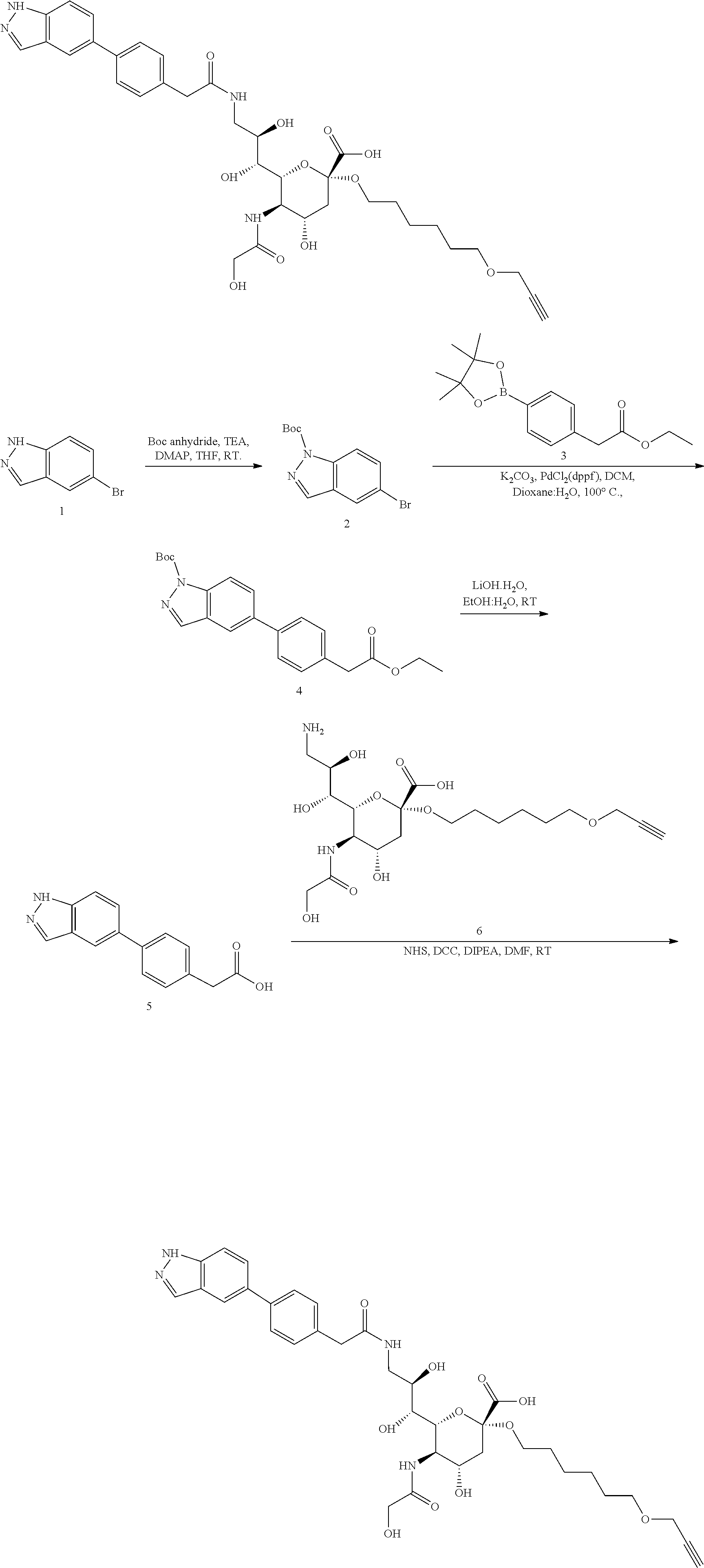

### Synthesis of tert-butyl 5-bromo-1H-indazole-1-carboxylate (2)

To a stirred solution of 5-bromo-1H-indazole (1, 1.0 g, 5.08 mmol) in tetrahydrofuran (10.0 mL) was added triethylamine (2.12 mL, 15.25 mmol), N,N-dimethyl-4-pyridylamine (0.310 g, 2.54 mmol) and tert-butoxy-tert-butoxycarbonylformylate (2.30 mL, 10.20 mmol) at 0° C. The reaction mixture was stirred at room temperature for 16 h. After completion, the reaction mixture was diluted with water and ethyl acetate. The organic layer was separated, dried, filtered, and concentrated. The crude compound was purified by silica gel column chromatography using ethyl acetate in heptane as eluents to afford the desired product tert-butyl 5-bromo-1H-indazole-1-carboxylate (2). Yield: 0.80 g, 53%; LCMS m/z 298.95 [M+H]⁺.

### Synthesis of tert-butyl 5-(4-(2-ethoxy-2-oxoethyl)phenyl)-1H-indazole-1-carboxylate (4)

A stirred solution of tert-butyl 5-bromo-1H-indazole-1-carboxylate (2, 1.00 g, 3.37 mmol) and ethyl 2-(4-(4,4,5,5-tetramethyl-1,3,2-dioxaborolan-2-yl)phenyl)acetate (3, 0.929 g, 3.37 mmol) and dipotassium carbonate (1.39 g, 10.11 mmol) in 1,4-dioxane (8.0 mL) and water (2.0 mL) was degassed by bubbling argon for 10 min. Pd(dppf)Cl₂·DCM (0.549 g, 0.674 mmol) was added at room temperature and the resulting mixture was stirred for 6 h at 100° C. After completion, the reaction was diluted with water and extracted with ethyl acetate. The organic layer was separated, dried, filtered, and concentrated. The crude residue obtained was purified by silica gel column chromatography using ethyl acetate in heptane as eluents to afford the desired product tert-butyl 5-(4-(2-ethoxy-2-oxoethyl)phenyl)-1H-indazole-1-carboxylate (4). Yield: 0.60 g, 47%; LCMS m/z 281.15 [M−100]⁺.

### Synthesis of 2-(4-(1H-indazol-5-yl)phenyl)acetic acid (5)

To a stirred solution of tert-butyl 5-(4-(2-ethoxy-2-oxoethyl)phenyl)-1H-indazole-1-carboxylate (4, 0.40 g, 1.05 mmol) in ethanol (4.0 mL) was added a solution of lithium hydroxide monohydrate (0.132 g, 3.15 mmol) in water (1.0 ml). The reaction mixture was stirred at room temperature for 6 h. After completion, the reaction mixture was treated with 1N HCl solution and extracted with ethyl acetate. The organic layer was concentrated to afford the desired product 2-(4-(1H-indazol-5-yl)phenyl)acetic acid (5) as a crude. Yield: 0.250 g, 94%; LCMS m/z 253.10 [M+H]⁺.

### Synthesis of (2R,4S,5R,6R)-6-((1R,2R)-3-(2-(4-(1H-indazol-5-yl)phenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Cpd. 2)

To a stirred solution of 2-(4-(1H-indazol-5-yl)phenyl)acetic acid (5, 0.100 g, 0.396 mmol) in N,N-dimethylformamide (3.0 mL), were successively added N,N′-dicyclohexylcarbodiimide (0.081 g, 0.396 mmol) and N-hydroxysuccinimide (0.045 g, 0.396 mmol) at 0° C. The reaction mixture was stirred at room temperature for 3 h. (2R,4S,5R,6R)-6-((1R,2R)-3-amino-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (0.183 g, 0.396 mmol) was added at 0° C. then stirred at room temperature for 4 h. After completion, the reaction mixture was concentrated, and the crude residue obtained was purified by prep HPLC using acetonitrile in water as eluents (+0.05% TFA) to afford the desired product (2R,4S,5R,6R)-6-((1R,2R)-3-(2-(4-(1H-indazol-5-yl)phenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Cpd. 2). Yield: 0.005 g, 2%; LCMS (ESI) m/z 697.40 [M+H]+; ¹H NMR (400 MHz, MeOD-d4): δ 8.09 (s, 1H), 7.98 (s, 1H), 7.68 (dd, J=8.4 Hz & 1.2 Hz, 1H), 7.63-7.59 (m, 3H), 7.40 (d, J=8.4 Hz, 2H), 4.12 (d, J=2.0 Hz, 2H), 4.01 (s, 2H), 3.90-3.87 (m, 1H), 3.83-3.77 (m, 2H), 3.75-3.67 (m, 3H), 3.59 (s, 2H), 3.56-3.50 (s, 1H), 3.45 (t, J=6.4 Hz, 2H), 3.39-3.37 (m, 2H), 3.25-3.22 (m, 1H), 2.80 (t, J=2.4 Hz, 1H), 2.74-2.72 (m, 1H), 1.69 (t, J=12.0 Hz, 1H), 1.60-1.51 (m, 5H), 1.38-1.23 (m, 5H).

### (2R,4S,5R,6R)-6-((1R,2R)-1,2-dihydroxy-3-(2-(4-(3-methyl-1H-indol-5-yl)phenyl)acetamido)propyl)-4-hydroxy-5-(2-hydroxyacetamido)-2-((6-(prop-2-yn-1-yloxy)hexyl)oxy)tetrahydro-2H-pyran-2-carboxylic acid (Compound 1)

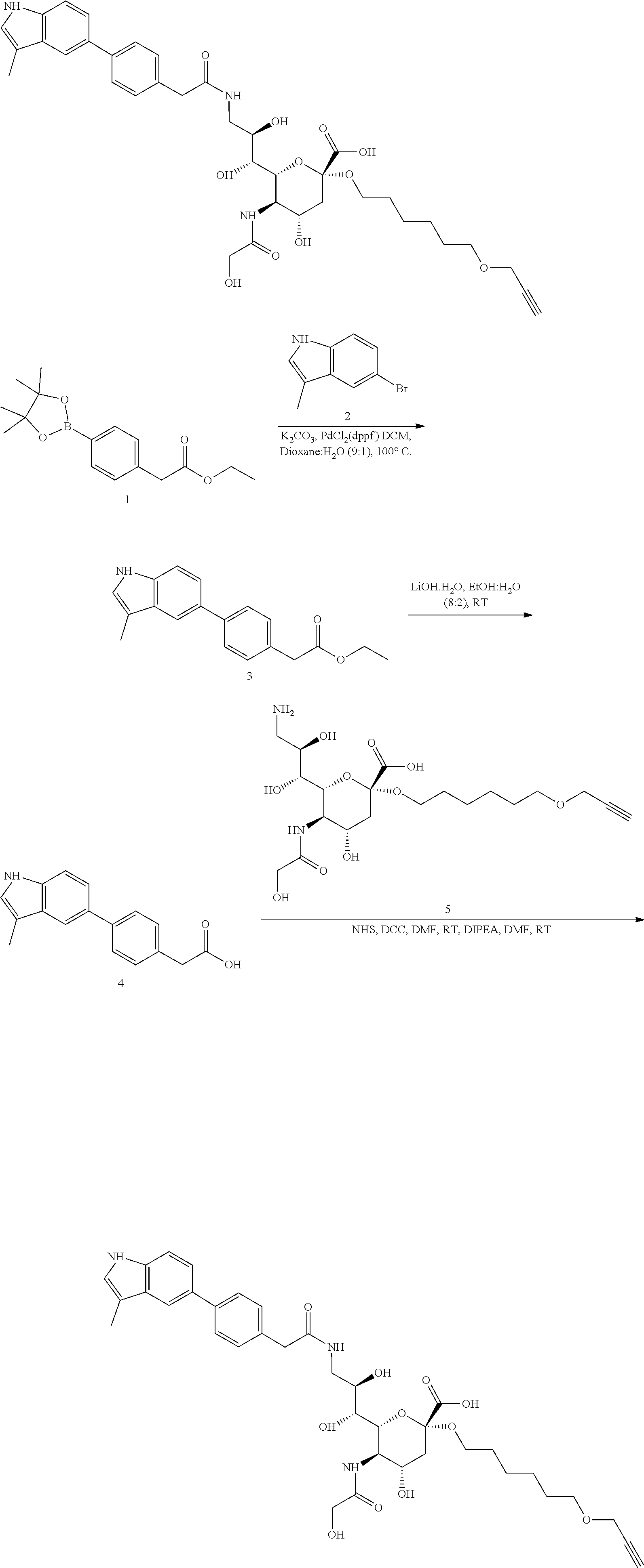

### Synthesis of ethyl 2-(4-(3-methyl-1H-indol-5-yl)phenyl)acetate (3)

A solution of 5-bromo-3-methyl-1H-indole (2, 1.00 g, 4.76 mmol), ethyl 2-(4-(4,4,5,5-tetramethyl-1,3,2-dioxaborolan-2-yl)phenyl)acetate (1, 1.66 g, 5.71 mmol) and dipotassium carbonate (1.97 g, 14.28 mmol) in 1,4-dioxane (8.0 mL) and water (2.0 mL) was degassed by bubbling argon gas for 10 min. Pd(dppf)Cl₂·DCM (0.388 g, 0.476 mmol) was added at room temperature and the mixture was stirred for 6 h at 100° C. After completion, the reaction was diluted with water and extracted by ethyl acetate. The organic layer was separated, dried, filtered, and concentrated. The crude residue obtained was purified by silica gel column chromatography using ethyl acetate in heptane as eluents to afford the desired product 2-(4-(3-methyl-1H-indol-5-yl)phenyl)acetate (3) Yield: 0.640 g, 46%; LCMS m/z 294.15 [M+H]⁺.

### Synthesis of 2-(4-(3-methyl-1H-indol-5-yl)phenyl)acetic acid (4)

To a stirred solution of 2-(4-(3-methyl-1H-indol-5-yl)phenyl)acetate (3, 0.640 g, 2.18 mmol) in Ethanol (6.0 mL) was added a solution of Lithium hydroxide monohydrate (0.281 g, 6.54 mmol) in water (2.0 ml). The reaction mixture was stirred at room temperature for 6 h. After completion, the reaction mixture was treated with 1N HCl solution and extracted with ethyl acetate. The organic layer was concentrated to afford the desired product 2-(4-(3-methyl-1H-indol-5-yl)phenyl)acetic acid (4). Yield: 0.40 g, 69%; LCMS m/z 266.10 [M+H]⁺.

### Synthesis of (2R,4S,5R,6R)-6-((1R,2R)-1,2-dihydroxy-3-(2-(4-(3-methyl-1H-indol-5-yl)phenyl)acetamido)propyl)-4-hydroxy-5-(2-hydroxyacetamido)-2-((6-(prop-2-yn-1-yloxy)hexyl)oxy)tetrahydro-2H-pyran-2-carboxylic acid (Cpd. 1)

To a stirred solution of 2-(4-(3-methyl-1H-indol-5-yl)phenyl)acetic acid (4, 0.100 g, 0.376 mmol) in N,N-dimethylformamide (3.0 mL) were successively added N,N′-dicyclohexylcarbodiimide (0.077 g, 0.376 mmol) and N-hydroxysuccinimide (0.043 g, 0.376 mmol) at 0° C., was stirred at room temperature for 3 h. (2R,4S,5R,6R)-6-((1R,2R)-3-amino-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (5, 0.174 g, 0.376 mmol) was added at 0° C. then stirred at room temperature for 4 h. After completion, the reaction mixture was concentrated, and the crude residue obtained was purified by prep HPLC using acetonitrile in water as eluents (+0.05% TFA) to afford the desired product (2R,4S,5R,6R)-6-((1R,2R)-1,2-dihydroxy-3-(2-(4-(3-methyl-1H-indol-5-yl)phenyl)acetamido)propyl)-4-hydroxy-5-(2-hydroxyacetamido)-2-((6-(prop-2-yn-1-yloxy)hexyl)oxy)tetrahydro-2H-pyran-2-carboxylic acid (Cpd. 1). Yield: 0.058 g, 22%; LCMS (ESI) m/z 710.50 [M+H]+; ¹H NMR (400 MHz, MeOD-d4): δ 7.71 (s, 1H), 7.62 (d, J=8.4 Hz, 2H), 7.39-7.37 (m, 4H), 7.03 (s, 1H), 4.10 (d, J=2.4 Hz, 2H), 4.04 (s, 1H), 3.92-3.86 (m, 1H), 3.85-3.81 (m, 2H), 3.79-3.69 (m, 3H), 3.64-3.56 (m, 2H), 3.47-3.44 (m, 2H), 3.42-3.38 (m, 2H), 3.29-3.24 (m, 1H), 2.82 (t, J=2.4 Hz, 1H), 2.73 (dd, J=12.4 Hz & 3.6 Hz, 1H), 2.36 (s, 3H), 1.73 (t, J=11.6 Hz, 1H), 1.53-1.48 (m, 4H), 1.37-1.31 (m, 4H).

### (2R,4S,5R,6R)-6-((1R,2R)-3-(2-(4-chlorophenyl)acetamido)-1,2-dihydroxypropyl)-2-(dec-9-yn-1-yloxy)-4-hydroxy-5-(2-hydroxyacetamido)tetrahydro-2H-pyran-2-carboxylic acid (Compound 109)

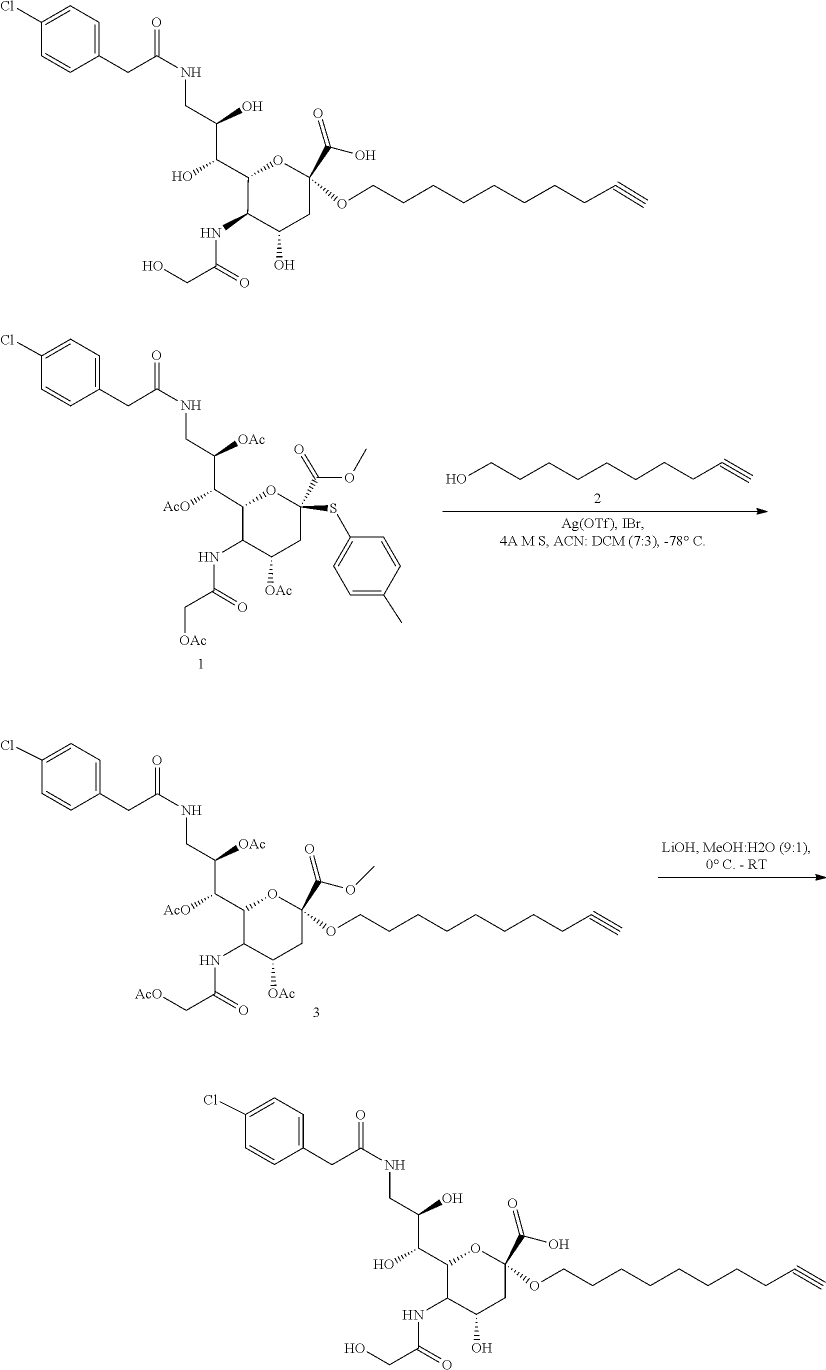

### Synthesis of (1R,2R)-1-((2R,3R,4S,6R)-4-acetoxy-3-(2-acetoxyacetamido)-6-(dec-9-yn-1-yloxy)-6-(methoxycarbonyl)tetrahydro-2H-pyran-2-yl)-3-(2-(4-chlorophenyl)acetamido)propane-1,2-diyl diacetate (3)

A solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (1, 0.40 g, 0.52 mmol) and dec-9-yn-1-ol (2, 0.48 g, 3.14 mmol), silver(1+) trifluoromethanesulfonate (0.40 g, 1.57 mmol) and activated 4 Å powdered molecular sieves (1.00 g) in anhydrous dichloromethane (115 mL) and anhydrous acetonitrile (180 mL) was stirred at room temperature for 1 h under nitrogen. The solution was cooled to −78° C. and iodobromane (0.22 g, 1.05 mmol) solution in dichloromethane (5.0 mL) was added dropwise and the reaction mixture was stirred at same temperature for 2 h. After completion the reaction mixture was quenched by addition of triethylamine (2.0 mL) and warmed to room temperature. The reaction mixture was filtered, the filtrate was washed with a saturated solution of sodium bicarbonate, dried over sodium sulfate, filtered and concentrated. The crude residue obtained was purified by silica-gel column chromatography using ethyl acetate in heptane as eluents to afford the desired product (1R,2R)-1-((2R,3R4S,6R)-4-acetoxy-3-(2-acetoxyacetamido)-6-(dec-9-yn-1-yloxy)-6-(methoxycarbonyl)tetrahydro-2H-pyran-2-yl)-3-(2-(4-chlorophenyl)acetamido)propane-1,2-diyl diacetate (3) as a white solid. Yield: 0.2 g, 48%; LCMS m/z 795.20 [M+H]⁺.

### Synthesis of (2R,4S,5R,6R)-6-((1R,2R)-3-(2-(4-chlorophenyl)acetamido)-1,2-dihydroxypropyl)-2-(dec-9-yn-1-yloxy)-4-hydroxy-5-(2-hydroxyacetamido)tetrahydro-2H-pyran-2-carboxylic acid (Cpd. 109)

To a stirred solution of (1R,2R)-1-((2R,3R,4S,6R)-4-acetoxy-3-(2-acetoxyacetamido)-6-(dec-9-yn-1-yloxy)-6-(methoxycarbonyl)tetrahydro-2H-pyran-2-yl)-3-(2-(4-chlorophenyl)acetamido)propane-1,2-diyl diacetate (3, 0.31 g, 0.42 mmol) in methanol (5 mL), was added solution of lithium hydroxide monohydrate (0.061 g, 2.64 mmol) in water (0.5 mL). The reaction mixture was stirred at room temperature for 6 h. After completion the reaction mixture was treated with acidic resin (Dowex 50, H⁺) to pH˜6 and the suspension was filtered. The filtrate was concentrated and the crude residue obtained was purified by prep HPLC using acetonitrile and water (+0.1% TFA) as eluents to afford the desired product (2R,4S,5R,6R)-6-((1R,2R)-3-(2-(4-chlorophenyl)acetamido)-1,2-dihydroxypropyl)-2-(dec-9-yn-1-yloxy)-4-hydroxy-5-(2-hydroxyacetamido)tetrahydro-2H-pyran-2-carboxylic acid (Cpd. 109) as an off white solid. Yield: 0.121 g, 80%; LCMS m/z 613.40 [M+H]⁺; ¹H NMR (400 MHz, Methanol-d₄) δ 7.32-7.29 (m, 4H), 4.01 (s, 2H), 3.89-3.68 (m, 5H), 3.65 (dd, J=14.0 Hz & 3.2 Hz, 1H), 3.53 (s, 2H), 3.43-3.35 (m, 2H), 3.26-3.21 (m, 1H), 2.72 (dd, J=12.4 Hz & 4.0 Hz, 1H), 2.17-2.14 (m, 3H), 1.73 (t, J=12.4 Hz, 1H), 1.54-1.47 (m, 4H), 1.43-1.32 (m, 8H).

### (2S,4S,5R,6R)-6-((1R,2R)-3-(2-(4-chlorophenyl)acetamido)-1,2-dihydroxypropyl)-4-hydroxy-5-(2-hydroxyacetamido)-2-((6-(prop-2-yn-1-yloxy)hexyl)thio)tetrahydro-2H-pyran-2-carboxylic acid (Compound 186)

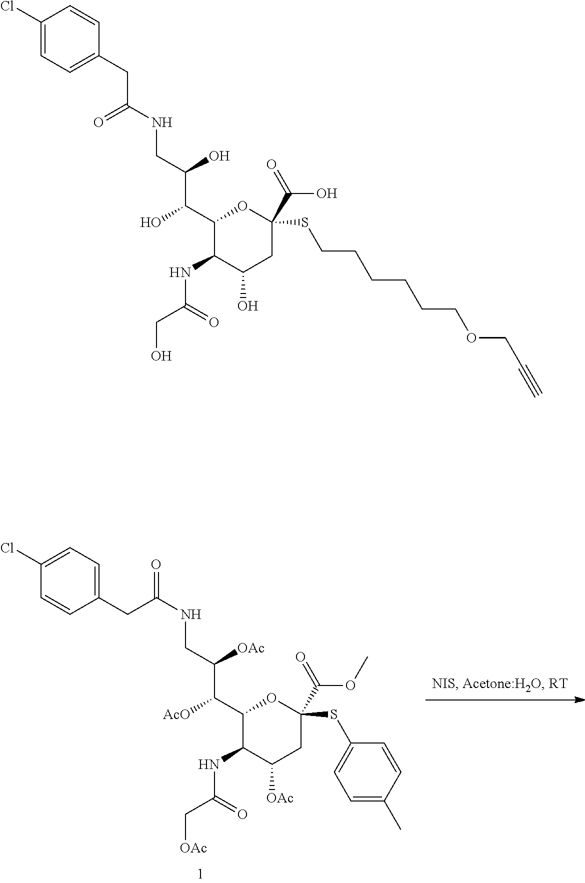 [SMILES: C#CCOCCCCCCS[C@]1(C(=O)O)C[C@H](O)[C@@H](NC(=O)CO)[C@H]([C@H](O)[C@H](O)CNC(=O)Cc2ccc(Cl)cc2)O1 ; COC(=O)[C@]1(Sc2ccc(C)cc2)C[C@H](OC(C)=O)[C@@H](NC(=O)COC(C)=O)[C@H]([C@H](OC(C)=O)[C@@H](CNC(=O)Cc2ccc(Cl)cc2)OC(C)=O)O1]

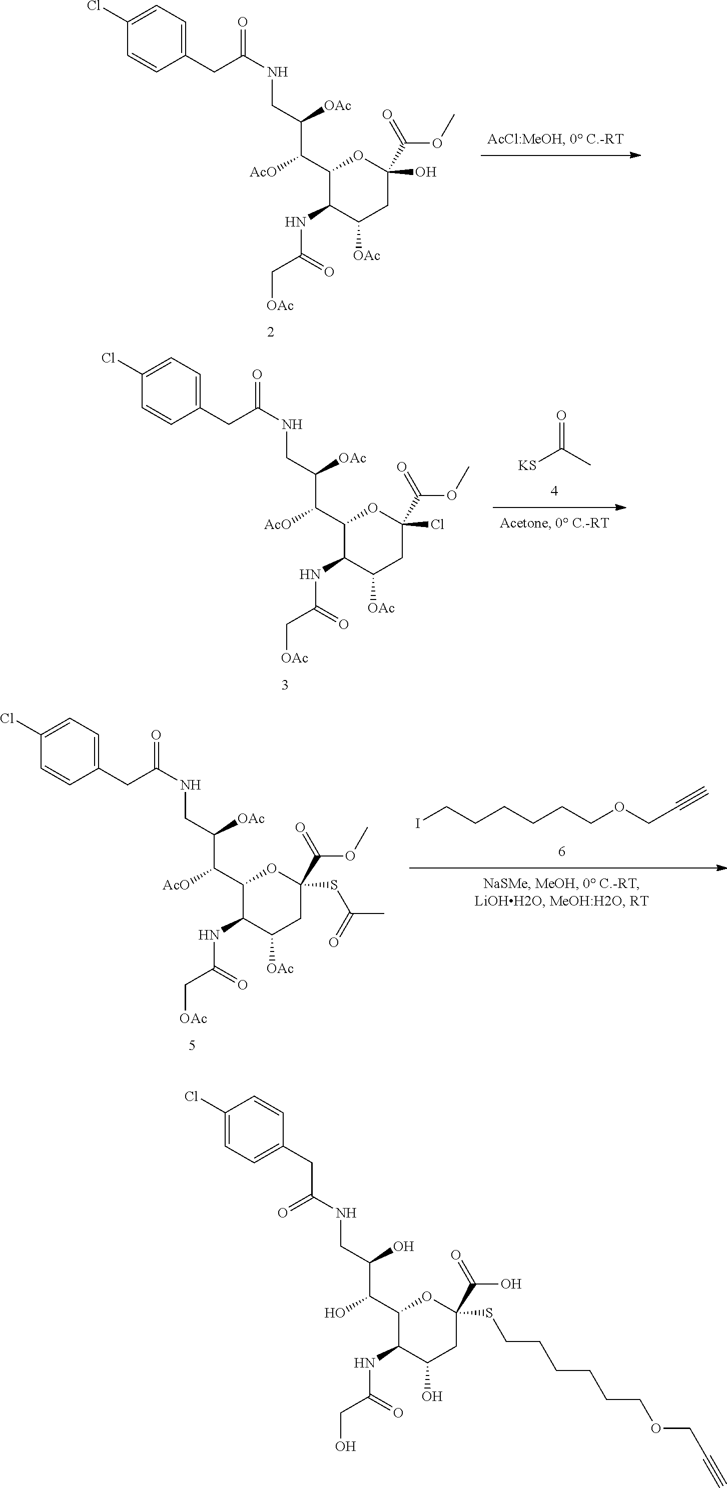

### Synthesis of (1R,2R)-1-((2R,3R,4S,6S)-4-acetoxy-3-(2-acetoxyacetamido)-6-hydroxy-6-(methoxycarbonyl)tetrahydro-2H-pyran-2-yl)-3-(2-(4-chlorophenyl)acetamido)propane-1,2-diyl diacetate (2)

To a solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (1, 2.50 g, 3.27 mmol) in acetone:water (9:1, 25.0 mL) at 0° C. was added N-iodosuccinimide (2.22 g, 9.90 mmol) was stirred at 0° C. for 3 h. After completion, saturated aqueous solution of sodium metabisulfide and ethyl acetate were added. The reaction mixture was stirred for another 10 min then the organic phase was separated. The aqueous phase was extracted with ethyl acetate, the organic layers were combined and washed sequentially with saturated sodium bicarbonate solution and water. The organic layer was dried and concentrated. The crude residue obtained was purified by silica gel column chromatography using ethyl acetate in heptane as eluents to afford the desired product (1R,2R)-1-((2R,3R,4S,6S)-4-acetoxy-3-(2-acetoxyacetamido)-6-hydroxy-6-(methoxycarbonyl)tetrahydro-2H-pyran-2-yl)-3-(2-(4-chlorophenyl)acetamido)propane-1,2-diyl diacetate (2). Yield: 1.40 g, 65%; LCMS (ESI) m/z 658.65 [M+H]⁺.

### Synthesis of (1R,2R)-1-((2R,3R,4S,6R)-4-acetoxy-3-(2-acetoxyacetamido)-6-chloro-6-(methoxycarbonyl)tetrahydro-2H-pyran-2-yl)-3-(2-(4-chlorophenyl)acetamido)propane-1,2-diyl diacetate (3)

To a stirred solution of (1R,2R)-1-((2R,3R,4S,6S)-4-acetoxy-3-(2-acetoxyacetamido)-6-hydroxy-6-(methoxycarbonyl)tetrahydro-2H-pyran-2-yl)-3-(2-(4-chlorophenyl)acetamido)propane-1,2-diyl diacetate (2, 1.40 g, 2.12 mmol) in acetyl chloride (75.0 mL) was added anhydrous methanol (3.0 mL) dropwise at 0° C. The resulting reaction mixture was stirred at room temperature for 24 h. After completion, the reaction mixture was concentrated to afford the desired product (1R,2R)-1-((2R,3R,4S,6R)-4-acetoxy-3-(2-acetoxyacetamido)-6-chloro-6-(methoxycarbonyl)tetrahydro-2H-pyran-2-yl)-3-(2-(4-chlorophenyl)acetamido)propane-1,2-diyl diacetate (3) as a crude which was used as such for the next step. Yield: 1.40 g, (crude).

### Synthesis of (1R,2R)-1-((2R,3R,4S,6S)-4-acetoxy-3-(2-acetoxyacetamido)-6-(acetylthio)-6-(methoxycarbonyl)tetrahydro-2H-pyran-2-yl)-3-(2-(4-chlorophenyl)acetamido)propane-1,2-diyl diacetate (5)

A solution of (1R,2R)-1-((2R,3R,4S,6R)-4-acetoxy-3-(2-acetoxyacetamido)-6-chloro-6-(methoxycarbonyl)tetrahydro-2H-pyran-2-yl)-3-(2-(4-chlorophenyl)acetamido)propane-1,2-diyl diacetate (3, 1.40 g, 2.07 mmol, crude) in dry acetone (15.0 mL) was cooled to 0° C. Potassium thioacetate (4, 0.708 g, 6.20 mmol) was added portion wise at 0° C. and the reaction was stirred for 3 h at 0° C. After completion, the mixture was concentrated. The crude was taken up in ethyl acetate and washed with water. The organic layer was separated, dried, filtered, and concentrated. The crude residue obtained was purified by silica gel column chromatography using ethyl acetate in heptane as eluents to afford the desired product (1R,2R)-1-((2R,3R,4S,6S)-4-acetoxy-3-(2-acetoxyacetamido)-6-(acetylthio)-6-(methoxycarbonyl)tetrahydro-2H-pyran-2-yl)-3-(2-(4-chlorophenyl)acetamido)propane-1,2-diyl diacetate (5). Yield: 1.00 g, 67%; LCMS (ESI) m/z 716.65 [M+H]⁺.

### Synthesis of (2S,4S,5R,6R)-6-((1R,2R)-3-(2-(4-chlorophenyl)acetamido)-1,2-dihydroxypropyl)-4-hydroxy-5-(2-hydroxyacetamido)-2-((6-(prop-2-yn-1-yloxy)hexyl)thio)tetrahydro-2H-pyran-2-carboxylic acid (Cpd. 186)

To a stirred solution of (1R,2R)-1-((2R,3R,4S,6S)-4-acetoxy-3-(2-acetoxyacetamido)-6-(acetylthio)-6-(methoxycarbonyl)tetrahydro-2H-pyran-2-yl)-3-(2-(4-chlorophenyl)acetamido)propane-1,2-diyl diacetate (5, 0.30 g, 0.418 mmol) in methanol (5.0 mL) was added sodium thiomethoxide (0.035 g, 0.502 mmol) at 0° C. and stirred for 1 h. To the reaction, 1-iodo-6-(prop-2-yn-1-yloxy)hexane (6, 0.222 g, 0.836 mmol) was added at 0° C. then stirred at room temperature for 1 h. After completion, a solution of lithium hydroxide (0.089 g, 2.09 mmol) in water (0.5 mL) was added at room temperature and the reaction was stirred at room temperature for 6 h. After completion, the mixture was treated with acidic resin (Dowex-hydrogen form) until pH˜6 and the suspension was filtered. The filtrate was concentrated, and the crude residue was purified with preparative HPLC using acetonitrile in water (+0.1% TFA) as eluents to afford the desired product (2S,4S,5R,6R)-6-((1R,2R)-3-(2-(4-chlorophenyl)acetamido)-1,2-dihydroxypropyl)-4-hydroxy-5-(2-hydroxyacetamido)-2-((6-(prop-2-yn-1-yloxy)hexyl)thio)tetrahydro-2H-pyran-2-carboxylic acid (186). Yield: 0.075 g, 28%; LCMS (ESI) m/z 631.35 [M+H]+. ¹H NMR (400 MHz, methanol-d₄): δ 7.32-7.27 (m, 4H), 4.12 (d, J=2.4 Hz, 2H), 4.00 (s, 2H), 3.89-3.78 (m, 3H), 3.66-3.59 (m, 2H), 3.53-3.49 (m, 4H), 3.35 (dd, J=9.2 Hz & 2.0 Hz, 1H), 3.25-3.20 (m, 1H), 2.80-2.73 (m, 3H), 2.65-2.58 (m, 1H), 1.77 (dd, J=12.8 Hz & 11.2 Hz, 1H), 1.59-1.53 (m, 4H), 1.37-1.35 (m, 4H).

### (2R,4S,5R,6R)-2-((15-carboxypentadecyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Compound 99)

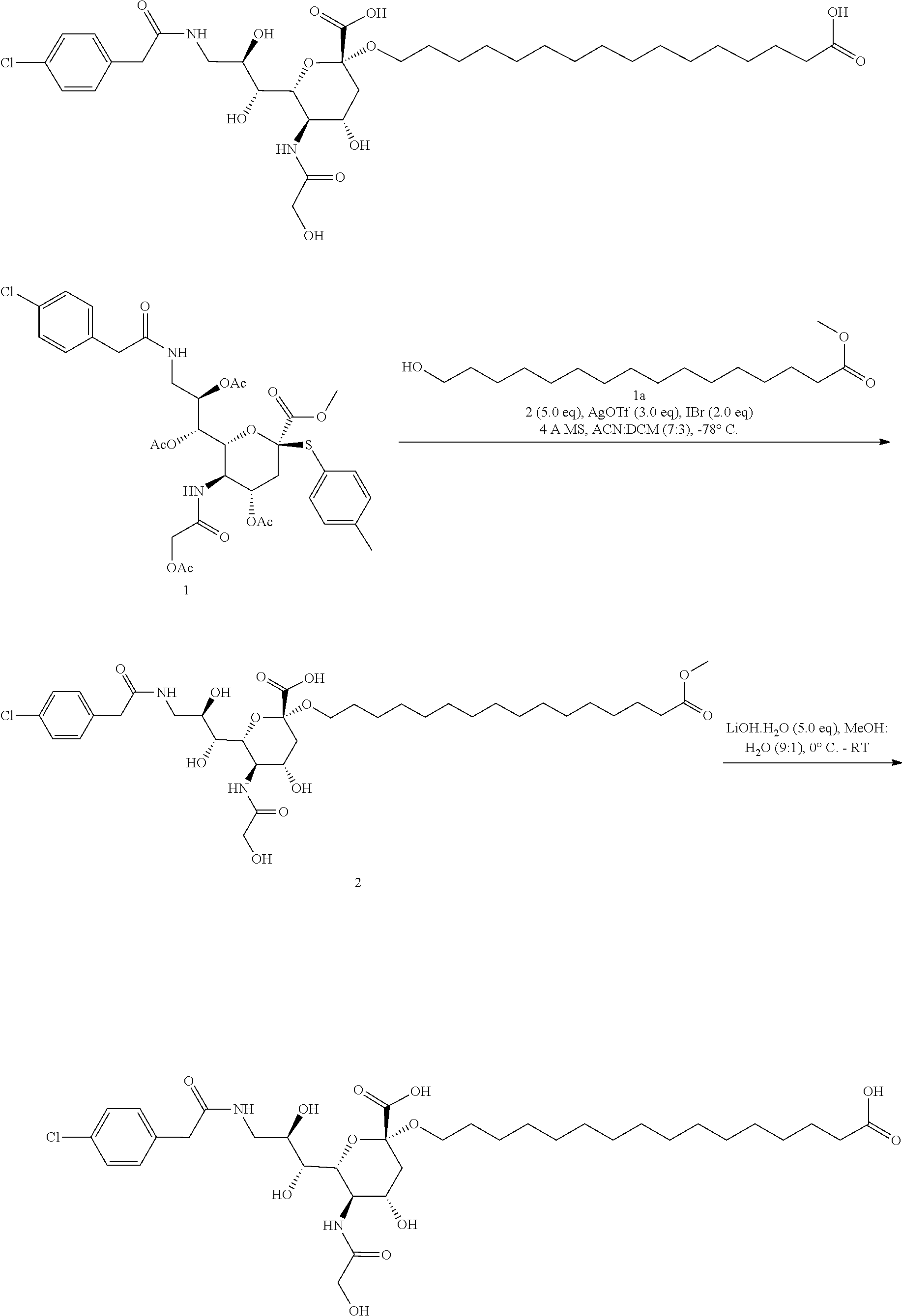 [SMILES: O=C(O)CCCCCCCCCCCCCCCO[C@]1(C(=O)O)C[C@H](O)[C@@H](NC(=O)CO)[C@H]([C@H](O)[C@H](O)CNC(=O)Cc2ccc(Cl)cc2)O1 ; COC(=O)[C@]1(Sc2ccc(C)cc2)C[C@H](OC(C)=O)[C@@H](NC(=O)COC(C)=O)[C@H]([C@H](OC(C)=O)[C@@H](CNC(=O)Cc2ccc(Cl)cc2)OC(C)=O)O1 ; COC(=O)CCCCCCCCCCCCCCCO ; COC(=O)CCCCCCCCCCCCCCCO[C@]1(C(=O)O)C[C@H](O)[C@@H](NC(=O)CO)[C@H]([C@H](O)[C@H](O)CNC(=O)Cc2ccc(Cl)cc2)O1 ; O=C(O)CCCCCCCCCCCCCCCO[C@]1(C(=O)O)C[C@H](O)[C@@H](NC(=O)CO)[C@H]([C@H](O)[C@H](O)CNC(=O)Cc2ccc(Cl)cc2)O1]

### Synthesis of (1R,2R)-1-((2R,3R,4S,6R)-4-acetoxy-3-(2-acetoxyacetamido)-6-((16-methoxy-16-oxohexadecyl)oxy)-6-(methoxycarbonyl)tetrahydro-2H-pyran-2-yl)-3-(2-(4-chlorophenyl)acetamido)propane-1,2-diyl diacetate (2)

A mixture of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (1, 0.4 g, 0.52 mmol), methyl 16-hydroxyhexadecanoate (1a, 0.75 g, 2.61 mmol), silver trifluoromethanesulfonate (AgOTf, 0.403 g, 1.57 mmol) and activated 4 Å powdered molecular sieves (1.0 g) in anhydrous dichloromethane (10 mL) and acetonitrile (15 mL) was stirred at room temperature for 2 h. A solution of Iodine mono bromide (IBr, 0.216 g, 1.05 mmol) in dichloromethane (1.0 mL) was added at −78° C. then stirred at the same temperature for 2 h. After completion, the reaction mixture was quenched by addition of triethylamine (0.2 mL) and warmed up to room temperature. The reaction mixture was filtered, and the filtrate was washed with aqueous saturated solution of sodium thiosulphate, dried, filtered, and concentrated. The crude residue obtained was purified by silica gel column chromatography using ethyl acetate in hexane as eluents to afford the desired product (1R,2R)-1-((2R,3R,4S,6R)-4-acetoxy-3-(2-acetoxyacetamido)-6-((16-methoxy-16-oxohexadecyl)oxy)-6-(methoxycarbonyl)tetrahydro-2H-pyran-2-yl)-3-(2-(4-chlorophenyl)acetamido)propane-1,2-diyl diacetate (2). Yield: 0.110 g, 22%; LCMS (ESI) m/z 927.10 [M+H]⁺.

### Synthesis of (2R,4S,5R,6R)-2-((15-carboxypentadecyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Cpd. 99)

To the stirred solution of (1R,2R)-1-((2R,3R,4S,6R)-4-acetoxy-3-(2-acetoxyacetamido)-6-((16-methoxy-16-oxohexadecyl)oxy)-6-(methoxycarbonyl)tetrahydro-2H-pyran-2-yl)-3-(2-(4-chlorophenyl)acetamido)propane-1,2-diyl diacetate (2, 0.200 g, 0.21 mmol) in methanol (5.0 mL) and water (0.5 mL), lithium hydroxide monohydrate (0.031 g, 1.29 mmol) was added at 0° C. The reaction mixture was stirred at room temperature for 3 h. After completion, the reaction mixture was quenched with acidic resin (Dowex-H⁺) until pH˜6. The suspension was filtered, and the filtrate was concentrated. The crude residue obtained was purified by HPLC using acetonitrile in water (+0.1% TFA) as eluents to afford the desired product (2R,4S,5R,6R)-2-((15-carboxypentadecyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Cpd. 99). Yield: 0.017 g, 11%; LCMS m/z 731.50 [M+H]+; ¹H-NMR (400 MHz, Methanol-d4): δ 8.05-8.02 (m, 1H), 7.90 (d, J=7.6 Hz, 1H), 7.31-7.27 (m, 4H), 4.02 (s, 2H), 3.89-3.63 (m, 6H), 3.53 (s, 2H), 3.40-3.36 (m, 2H), 3.26-3.20 (m, 1H), 2.74-2.71 (m, 1H), 2.27 (t, J=7.6 Hz, 2H), 1.74-1.68 (m, 1H), 1.61-1.52 (m, 4H), 1.29 (m, 22H).

### (2S,4S,5R,6R)-6-((1R,2R)-1,2-dihydroxy-3-(2-(3-hydroxyphenyl)acetamido)propyl)-4-hydroxy-5-(2-hydroxyacetamido)-2-(oct-7-yn-1-ylthio)tetrahydro-2H-pyran-2-carboxylic acid (Compound 141)

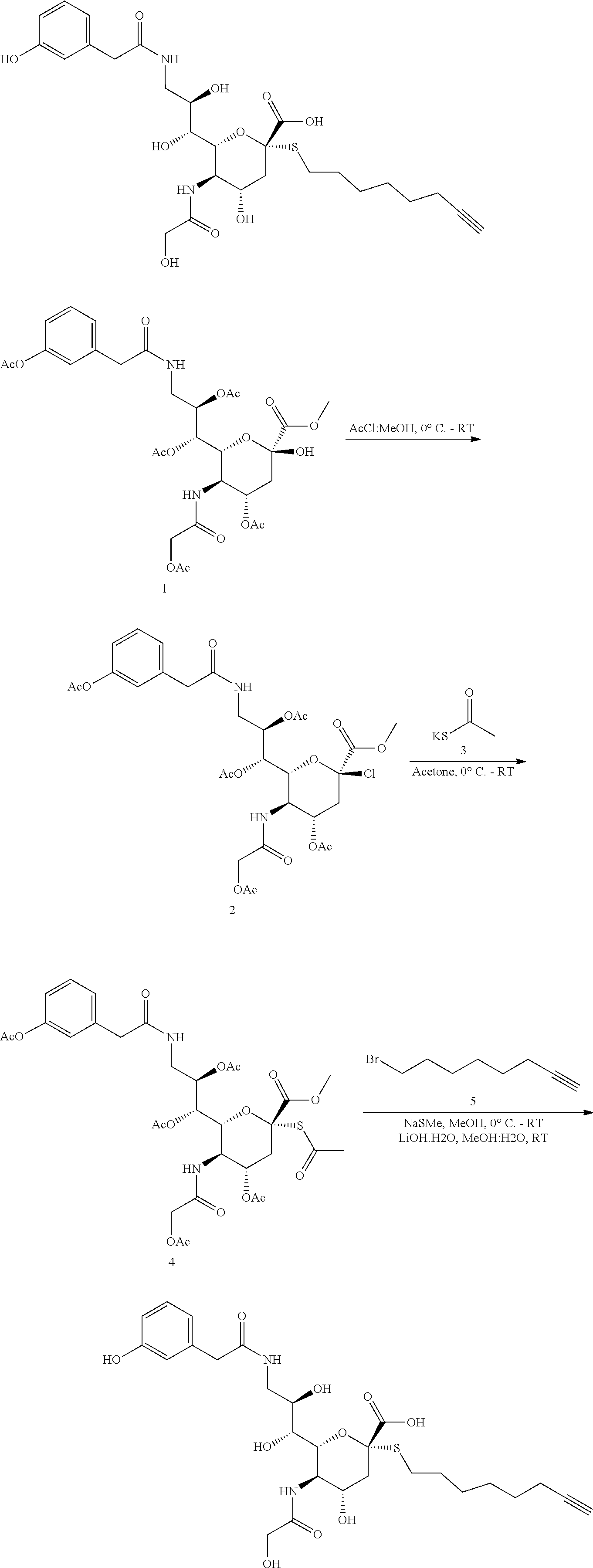

### Synthesis of (1R,2R)-1-((2R,3R,4S,6R)-4-acetoxy-3-(2-acetoxyacetamido)-6-chloro-6-(methoxycarbonyl)tetrahydro-2H-pyran-2-yl)-3-(2-(3-acetoxyphenyl)acetamido)propane-1,2-diyl diacetate (2)

To a stirred solution of (1R,2R)-1-((2R,3R,4S,6S)-4-acetoxy-3-(2-acetoxyacetamido)-6-hydroxy-6-(methoxycarbonyl)tetrahydro-2H-pyran-2-yl)-3-(2-(3-acetoxyphenyl)acetamido)propane-1,2-diyl diacetate (1, 0.60 g, 0.878 mmol) in acetyl chloride (30.0 mL) was added anhydrous methanol (1.5 mL) dropwise at 0° C. The resulting reaction mixture was stirred at room temperature for 24 h. After completion, the reaction mixture was concentrated to afford the desired product (1R,2R)-1-((2R,3R,4S,6R)-4-acetoxy-3-(2-acetoxyacetamido)-6-chloro-6-(methoxycarbonyl)tetrahydro-2H-pyran-2-yl)-3-(2-(3-acetoxyphenyl)acetamido)propane-1,2-diyl diacetate (2) as a crude which was used as such for the next step. Yield: 0.60 g, (crude).

### Synthesis of (1R,2R)-1-((2R,3R,4S,6S)-4-acetoxy-3-(2-acetoxyacetamido)-6-(acetylthio)-6-(methoxycarbonyl)tetrahydro-2H-pyran-2-yl)-3-(2-(3-acetoxyphenyl)acetamido)propane-1,2-diyl diacetate (4)

The crude previously obtained (1R,2R)-1-((2R,3R,4S,6R)-4-acetoxy-3-(2-acetoxyacetamido)-6-chloro-6-(methoxycarbonyl)tetrahydro-2H-pyran-2-yl)-3-(2-(3-acetoxyphenyl)acetamido)propane-1,2-diyl diacetate (2, 0.60 g, 0.855 mmol, crude) was dissolved under stirring in dry acetone (10.0 mL) and cooled at 0° C. Potassium thioacetate (3, 0.293 g, 2.57 mmol) was added portion wise at 0° C. and the reaction was stirred for 3 h at 0° C. After completion the mixture was concentrated. The crude was dissolved in ethyl acetate and washed with water. The organic layer was separated, dried and concentrated. The crude residue obtained was purified by silica gel column chromatography using ethyl acetate in heptane as eluents to afford the desired product (1R,2R)-1-((2R,3R,4S,6S)-4-acetoxy-3-(2-acetoxyacetamido)-6-(acetylthio)-6-(methoxycarbonyl)tetrahydro-2H-pyran-2-yl)-3-(2-(3-acetoxyphenyl)acetamido)propane-1,2-diyl diacetate (4). Yield: 0.40 g, 63%; LCMS (ESI) m/z 741.05 [M+H]⁺.

### Synthesis of (2S,4S,5R,6R)-6-((1R,2R)-1,2-dihydroxy-3-(2-(3-hydroxyphenyl)acetamido)propyl)-4-hydroxy-5-(2-hydroxyacetamido)-2-(oct-7-yn-1-ylthio)tetrahydro-2H-pyran-2-carboxylic acid (Cpd. 141)

To a stirred solution of (1R,2R)-1-((2R,3R,4S,6S)-4-acetoxy-3-(2-acetoxyacetamido)-6-(acetylthio)-6-(methoxycarbonyl)tetrahydro-2H-pyran-2-yl)-3-(2-(3-acetoxyphenyl)acetamido)propane-1,2-diyl diacetate (4, 0.20 g, 0.270 mmol) in methanol (5.0 mL) was added sodium thiomethoxide (0.0276 g, 0.324 mmol) at 0° C. and stirred for 1 h. 8-Bromooct-1-yne (5, 0.153 g, 0.810 mmol) was added at 0° C. then stirred at room temperature for 1 h. After completion, a solution of lithium hydroxide (0.069 g, 1.62 mmol) in water (0.5 mL) was added at room temperature and the reaction was stirred at room temperature for 6 h. After completion, the mixture was treated with acidic resin (Dowex H form) to reach pH 6 and the reaction was filtered. The filtrate was concentrated. The crude residue obtained was purified with preparative HPLC using acetonitrile in water (+0.1% TFA) as eluents to afford the desired product (2S,4S,5R,6R)-6-((1R,2R)-1,2-dihydroxy-3-(2-(3-hydroxyphenyl)acetamido)propyl)-4-hydroxy-5-(2-hydroxyacetamido)-2-(oct-7-yn-1-ylthio)tetrahydro-2H-pyran-2-carboxylic acid (141). Yield: 0.058 g, 37%; LCMS (ESI) m/z 583.40 [M+H]+. ¹H NMR (400 MHz, methanol-d₄): δ 7.11 (t, J=8.0 Hz, 1H), 6.76-6.73 (m, 2H), 6.66 (dd, J=8.0 Hz & 2.0 Hz, 1H), 4.01 (s, 2H), 3.87-3.80 (m, 3H), 3.66-3.59 (m, 2H), 3.47 (s, 2H), 3.35 (dd, J=8.8 Hz & 1.6 Hz, 1H), 3.26-3.21 (m, 1H), 2.79-2.72 (m, 2H), 2.66-2.59 (m, 1H), 2.18-2.14 (m, 3H), 1.77 (t, J=12.4 Hz, 1H), 1.63-1.44 (m, 4H), 1.39-1.29 (4H).

### (2R,4S,5R,6R)-6-((1R,2R)-3-(2-(4′-acetamido-[1,1′-biphenyl]-4-yl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Compound 204)

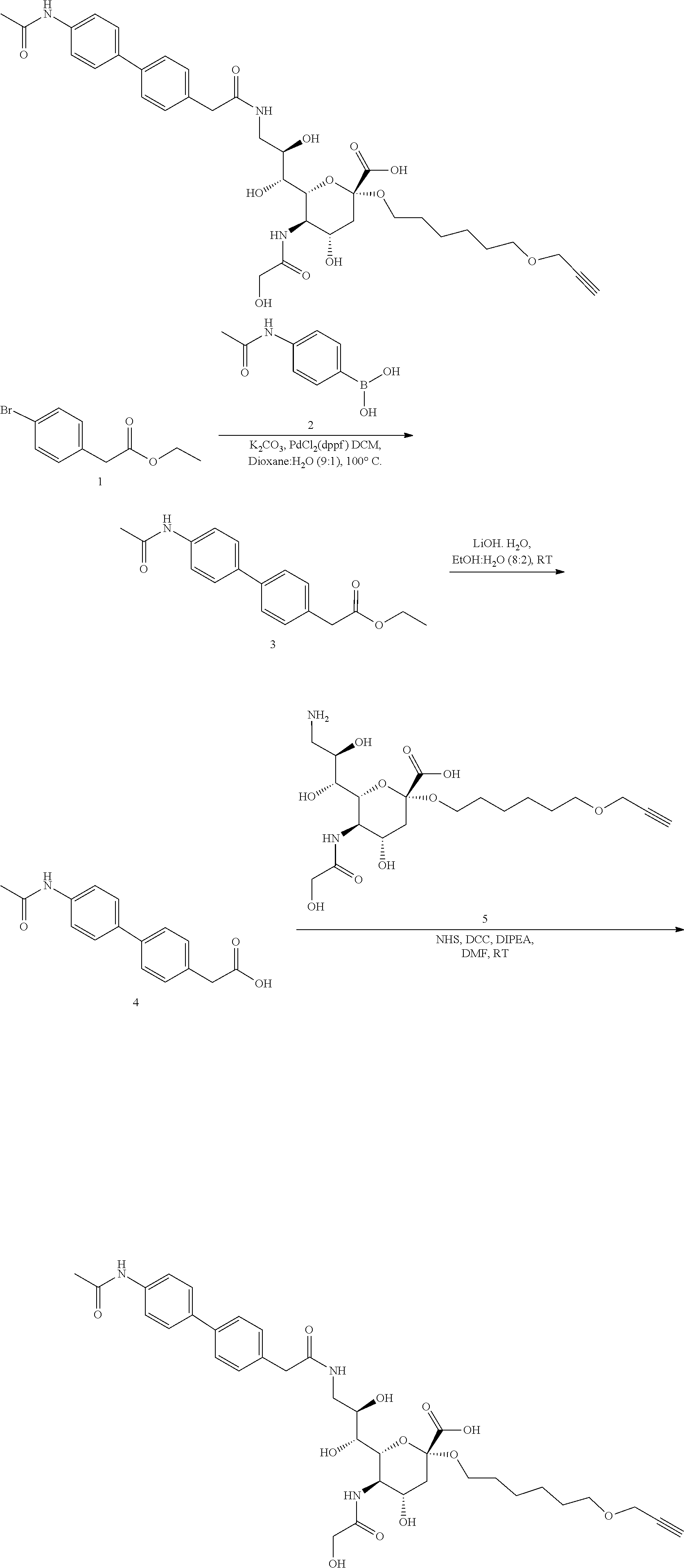

### Synthesis of ethyl 2-(4′-acetamido-[1,1′-biphenyl]-4-yl)acetate (3)

A stirred solution of ethyl 2-(4-bromophenyl)acetate (1, 1.00 g, 4.11 mmol) and (4-acetamidophenyl)boronic acid (2, 0.883 g, 4.94 mmol) and dipotassium carbonate (1.704 g, 12.83 mmol) in 1,4-dioxane (8.0 mL) and water (2.0 mL) was degassed by bubbling argon gas for 10 min. Pd(dppf)Cl₂·DCM (0.335 g, 0.411 mmol) was added at room temperature and the resulting mixture was stirred for 6 h at 100° C. After completion, the reaction mixture was diluted with water and extracted by ethyl acetate. The organic layer was separated, dried, filtered, and concentrated. The crude residue obtained was purified by silica gel column chromatography using ethyl acetate in heptane as eluents to afford the desired product ethyl 2-(4′-acetamido-[1,1′-biphenyl]-4-yl)acetate (3). Yield: 0.600 g, 49%; LCMS m/z 298.10 [M+H]⁺.

### Synthesis of 2-(4′-acetamido-[1,1′-biphenyl]-4-yl)acetic acid (4)

To a stirred solution of ethyl 2-(4′-acetamido-[1,1′-biphenyl]-4-yl)acetate (3, 0.600 g, 2.02 mmol) in Ethanol (6.0 mL) was added a solution of Lithium hydroxide monohydrate (0.260 g, 6.06 mmol) in water (2.0 ml). The reaction mixture was stirred at room temperature for 6 h. After completion, the reaction mixture was treated with 1N HCl solution and extracted with ethyl acetate. The organic layer was concentrated to afford the desired product 2-(4′-acetamido-[1,1′-biphenyl]-4-yl)acetic acid (4). Yield: 0.45 g, 83%; LCMS (m/z) 270.10 [M+H]⁺.

### Synthesis of (2R,4S,5R,6R)-6-((1R,2R)-3-(2-(4′-acetamido-[1,1′-biphenyl]-4-yl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Cpd. 204)

To a stirred solution of 2-(4′-acetamido-[1,1′-biphenyl]-4-yl)acetic acid (4, 0.130 g, 0.482 mmol) in N,N-dimethylformamide (3.0 mL), were successively added N,N′-dicyclohexylcarbodiimide (0.098 g, 0.482 mmol) and N-hydroxysuccinimide (0.055 g, 0.482 mmol) at 0° C. The reaction mixture was stirred at room temperature for 3 h. (2R,4S,5R,6R)-6-((1R,2R)-3-amino-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (5, 0.223 g, 0.482 mmol) was added at 0° C. then stirred at room temperature for another 4 h. After completion, the reaction mixture was concentrated, and the crude residue obtained was purified by prep HPLC using acetonitrile in water (+0.1% TFA) as eluents to afford the desired product (2R,4S,5R,6R)-6-((1R,2R)-3-(2-(4′-acetamido-[1,1′-biphenyl]-4-yl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Cpd. 204). Yield: 0.033 g, 10%; LCMS (ESI) m/z 714.50 [M+H]+; ¹H NMR (400 MHz, MeOD-d4): δ 7.89 (d, J=8 Hz, 1H), 7.63-7.61 (m, 2H), 7.57-7.51 (m, 4H), 7.36 (d, J=8.0 Hz, 2H), 4.10 (d, J=2.4 Hz, 2H), 4.01 (s, 2H), 3.90-3.85 (m, 1H), 3.83-3.78 (m, 2H), 3.76-3.72 (m, 1H), 3.72-3.65 (m, 2H), 3.57 (s, 2H), 3.48 (t, J=6.4 Hz, 2H), 3.40-3.36 (m, 2H), 3.27-3.22 (m, 1H), 2.81 (t, J=2.4 Hz, 1H), 2.70 (dd, J=12.4 Hz & 3.6 Hz, 1H), 2.14 (s, 3H), 1.73 (t, J=12.4 Hz, 1H), 1.53-1.44 (m, 4H), 1.34-1.32 (m, 4H).

### (2R,4S,5R,6R)-6-((1R,2R)-3-(2-(4-chloro-3-hydroxyphenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Compound 128)

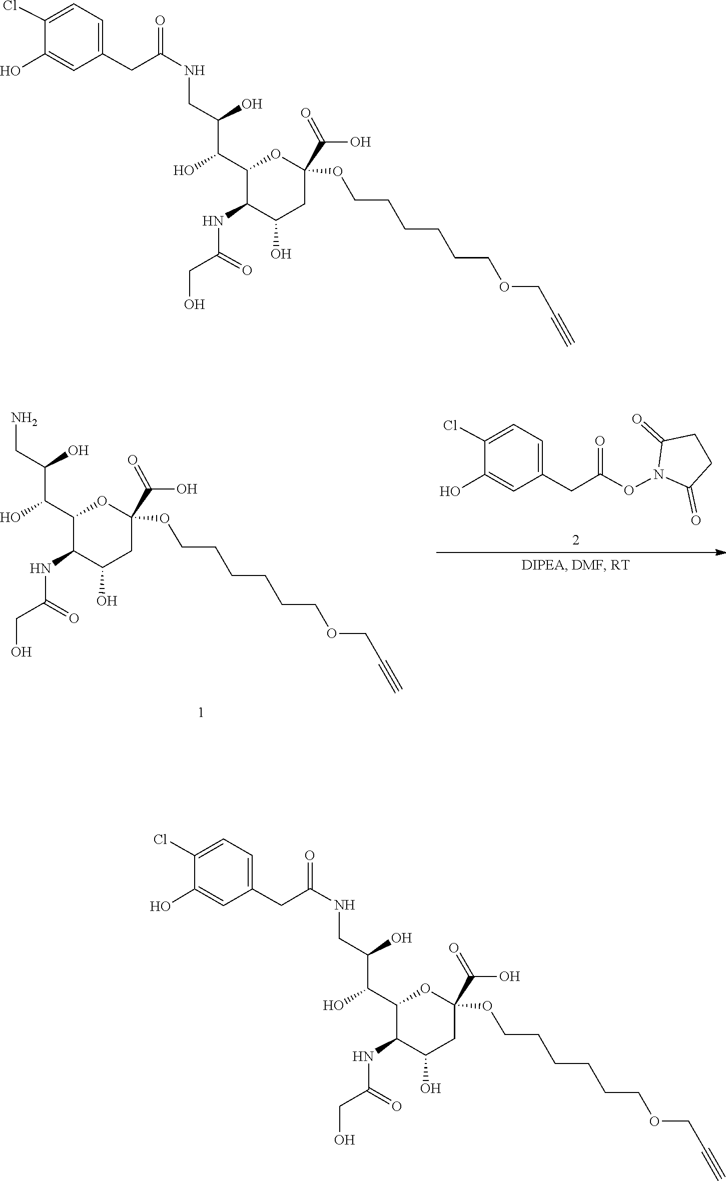 [SMILES: C#CCOCCCCCCO[C@]1(C(=O)O)C[C@H](O)[C@@H](NC(=O)CO)[C@H]([C@H](O)[C@H](O)CNC(=O)Cc2ccc(Cl)c(O)c2)O1 ; C#CCOCCCCCCO[C@]1(C(=O)O)C[C@H](O)[C@@H](NC(=O)CO)[C@H]([C@H](O)[C@H](O)CN)O1 ; O=C(Cc1ccc(Cl)c(O)c1)ON1C(=O)CCC1=O ; C#CCOCCCCCCO[C@]1(C(=O)O)C[C@H](O)[C@@H](NC(=O)CO)[C@H]([C@H](O)[C@H](O)CNC(=O)Cc2ccc(Cl)c(O)c2)O1]

### Synthesis of (2R,4S,5R,6R)-6-((1R,2R)-3-(2-(4-chloro-3-hydroxyphenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Cpd. 128)

To a stirred solution of (2R,4S,5R,6R)-6-((1R,2R)-3-amino-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (1, 0.100 g, 0.216 mmol) in N,N-dimethylformamide (2.0 mL) were added N,N-diisopropylethylamine (0.188 mL, 1.08 mmol) and 2,5-dioxopyrrolidin-1-yl 2-(4-chloro-3-hydroxyphenyl)acetate (2, 0.061 g, 0.216 mmol) at 0° C. then stirred at room temperature for 4 h. After completion, the reaction mixture was concentrated, and the crude residue obtained was purified by prep HPLC using acetonitrile in water (+0.1% TFA) as eluents to afford the desired product (2R,4S,5R,6R)-6-((1R,2R)-3-(2-(4-chloro-3-hydroxyphenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Cpd. 128). Yield: 0.033 g, 24%; LCMS (ESI) m/z 631.40 [M+H]+; ¹H NMR (400 MHz, MeOD-d₄): δ 7.20 (d, J=8.4 Hz, 1H), 6.86 (d, J=2.0 Hz, 1H), 6.74 (dd, J=9.2 Hz & 2.0 Hz, 1H), 4.12 (d, J=2.4 Hz, 2H), 4.01 (s, 2H), 3.90-3.69 (m, 5H), 3.64 (dd, J=14.0 Hz & 3.2 Hz, 1H), 3.51 (t, J=6.8 Hz, 2H), 3.45 (s, 2H), 3.41-3.36 (m, 2H), 3.26-3.21 (m, 1H), 2.81 (t, J=2.4 Hz, 1H), 2.72 (dd, J=12.8 Hz & 3.6 Hz, 1H), 1.71 (t, J=12.4 Hz, 1H), 1.57-1.54 (In, 4H), 1.40-1.35 (n, 4H).

### (2R,4S,5R,6R)-6-((1R,2R)-3-(2-(4-chloro-3-fluorophenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Compound 130)

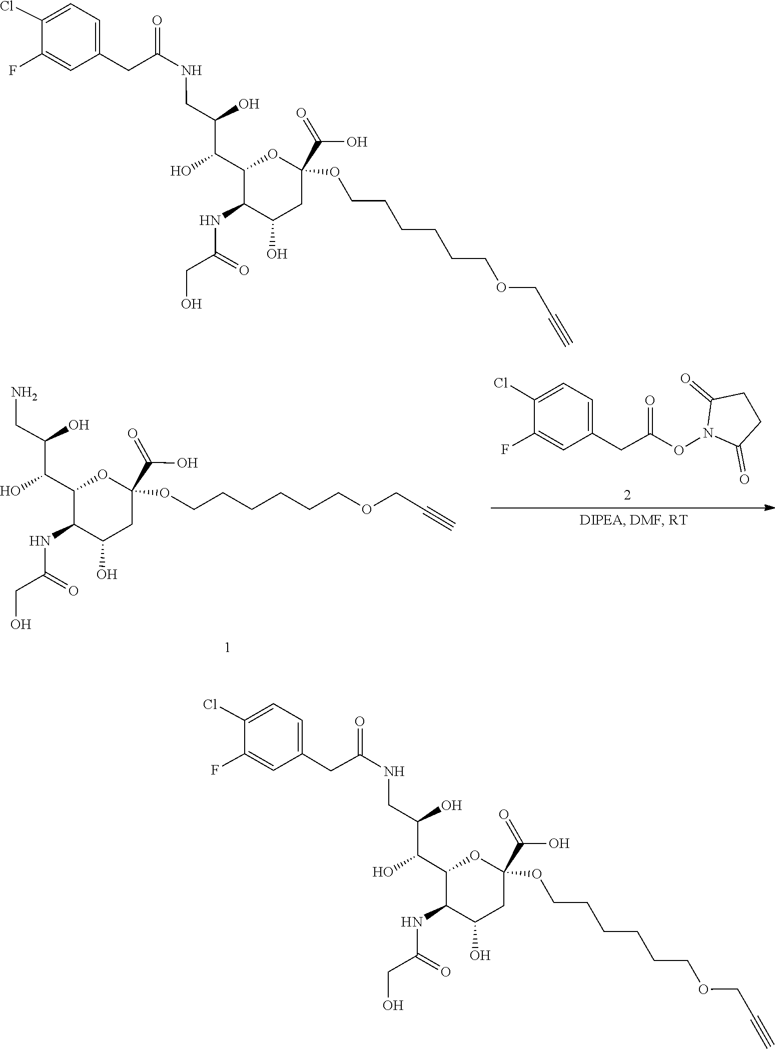

### Synthesis of (2R,4S,5R,6R)-6-((1R,2R)-3-(2-(4-chloro-3-fluorophenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Cpd. 130)

To a stirred solution of (2R,4S,5R,6R)-6-((1R,2R)-3-amino-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (1, 0.100 g, 0.216 mmol) and 2,5-dioxopyrrolidin-1-yl 2-(4-chloro-3-fluorophenyl)acetate (2, 0.049 g, 0.172 mmol) in N,N-dimethylformamide (2.0 mL) was added ethylbis(propan-2-yl)amine (0.18 mL, 1.08 mmol) at 0° C. then stirred at room temperature for 4 h. After completion, the reaction mixture was concentrated. The crude residue obtained was purified by prep HPLC using acetonitrile in water (+0.1% TFA) as eluents to afford the desired product (2R,4S,5R,6R)-6-((1R,2R)-3-(2-(4-chloro-3-fluorophenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Cpd. 130). Yield: 0.039 g; 29%; LCMS (ESI) m/z 633.25 [M+H]+. AH NMR (400 MHz, MeOD-d4): δ 7.39 (t, J=8.0 Hz, 1H), 7.21 (dd, J=10.4 Hz & 2.0 Hz, 1H), 7.11 (d, J=8.4 Hz, 1H), 4.12 (d, J=2.0 Hz, 2H), 4.01 (s, 2H), 3.87 (td, J=11.6 Hz & 3.2 Hz, 1H), 3.84-3.64 (m, 5H), 3.54 (s, 2H), 3.51 (t, J=6.4 Hz, 2H), 3.42-3.36 (m, 2H), 3.26-3.20 (m, 1H), 2.81 (t, J=2.4 Hz, 1H), 2.71 (dd, J=12.8 Hz & 4.0 Hz, 1H), 1.74 (t, J=5.6 Hz, 1H), 1.57-1.53 (m, 4H), 1.39-1.35 (in, 4H).

### (2R,4S,5R,6R)-6-((1R,2R)-3-(2-(4-(4-acetamidopiperidin-1-yl)phenyl)acetamido)-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (Compound 137)

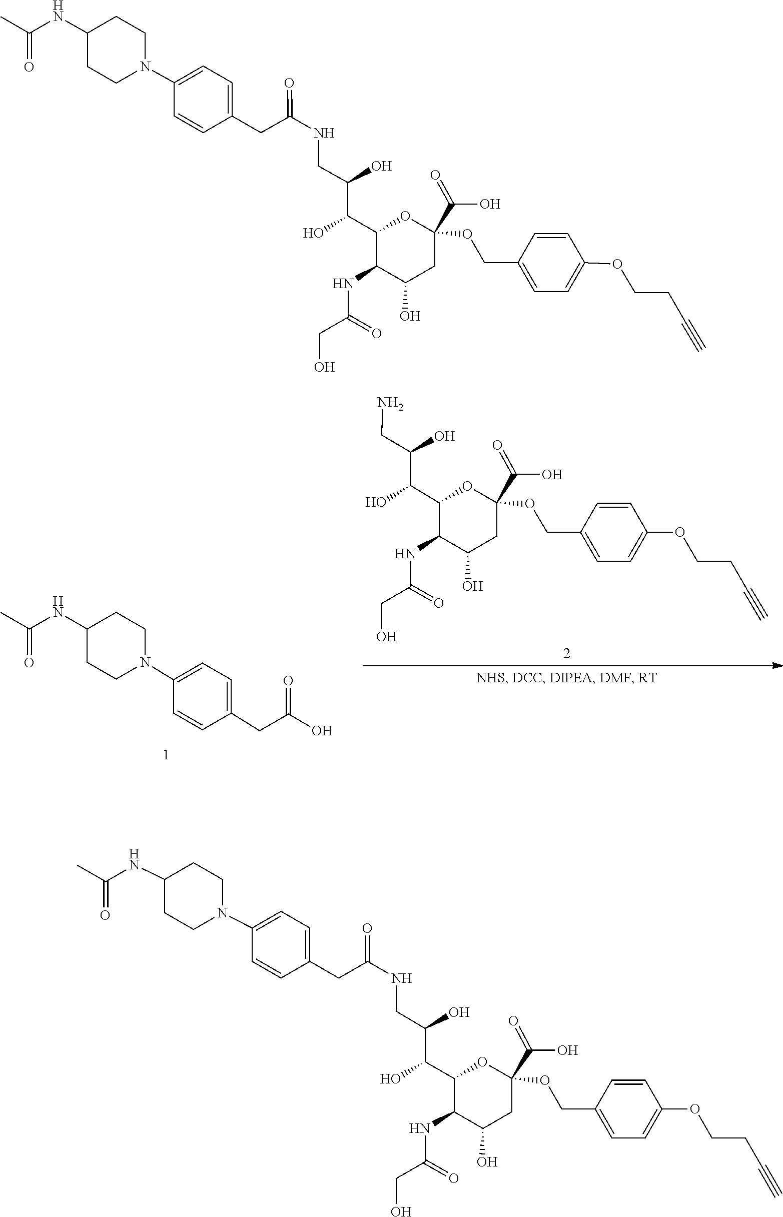

### Synthesis of (2R,4S,5R,6R)-6-((1R,2R)-3-(2-(4-(4-acetamidopiperidin-1-yl)phenyl)acetamido)-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (Cpd. 137)

To a stirred solution of 2-(4-(4-acetamidopiperidin-1-yl)phenyl)acetic acid (1, 0.06 g, 0.22 mmol) in N,N-dimethylformamide (3.0 mL) were successively added N,N′-dicyclohexylcarbodiimide (0.045 g, 0.22 mmol) and N-hydroxysuccinimide (0.025 g, 0.22 mmol) at 0° C. The reaction mixture was stirred at room temperature for 3 h. To that reaction mixture (2R,4S,5R,6R)-6-((1R,2R)-3-(2-(4-(4-acetamidopiperidin-1-yl)phenyl)acetamido)-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (2, 0.10 g, 0.22 mmol) in N,N-dimethylformamide (1.0 mL) was added at 0° C. then stirred at room temperature for 4 h. After completion, the reaction mixture was concentrated, and the crude residue obtained was purified by prep HPLC using acetonitrile in water (+0.1% TFA) as eluents to afford the desired product (2R,4S,5R,6R)-6-((1R,2R)-3-(2-(4-(4-acetamidopiperidin-1-yl)phenyl)acetamido)-1,2-dihydroxypropyl)-2-((4-(but-3-yn-1-yloxy)benzyl)oxy)-4-hydroxy-5-(2-hydroxyacetamido)tetrahydro-2H-pyran-2-carboxylic acid (Cpd. 137). Yield: 0.010 g, 6.22%; LCMS (ESI) m/z 741.45 [M+H]+; ¹H NMR (400 MHz, Methanol-d₄): δ 7.25 (d, J=8.4 Hz, 4H), 7.06 (d, J=8.4 Hz, 2H), 6.88 (d, J=8.4 Hz, 2H), 4.73 (d, J=10.8 Hz, 1H), 4.41 (d, J=10.8 Hz, 1H), 4.07 (t, J=6.8 Hz, 2H), 4.03 (s, 2H), 3.93-3.76 (m, 5H), 3.69-3.66 (m, 1H), 3.58 (d, J=12.4 Hz, 2H), 3.49 (s, 2H), 3.40-3.37 (m, 2H), 3.24-3.21 (m, 2H), 2.95 (t, J=12.0 Hz, 2H), 2.80 (dd, J=12.4 Hz & 3.6 Hz, 1H), 2.64 (td, J=6.8 Hz & 2.4 Hz, 2H), 2.33 (s, 1H), 1.99 (d, J=12.0 Hz, 2H), 1.95 (s, 3H), 1.78-1.64 (m, 3H).

### (2R,4S,5R,6R)-2-((6-carboxyhexyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Compound 98)

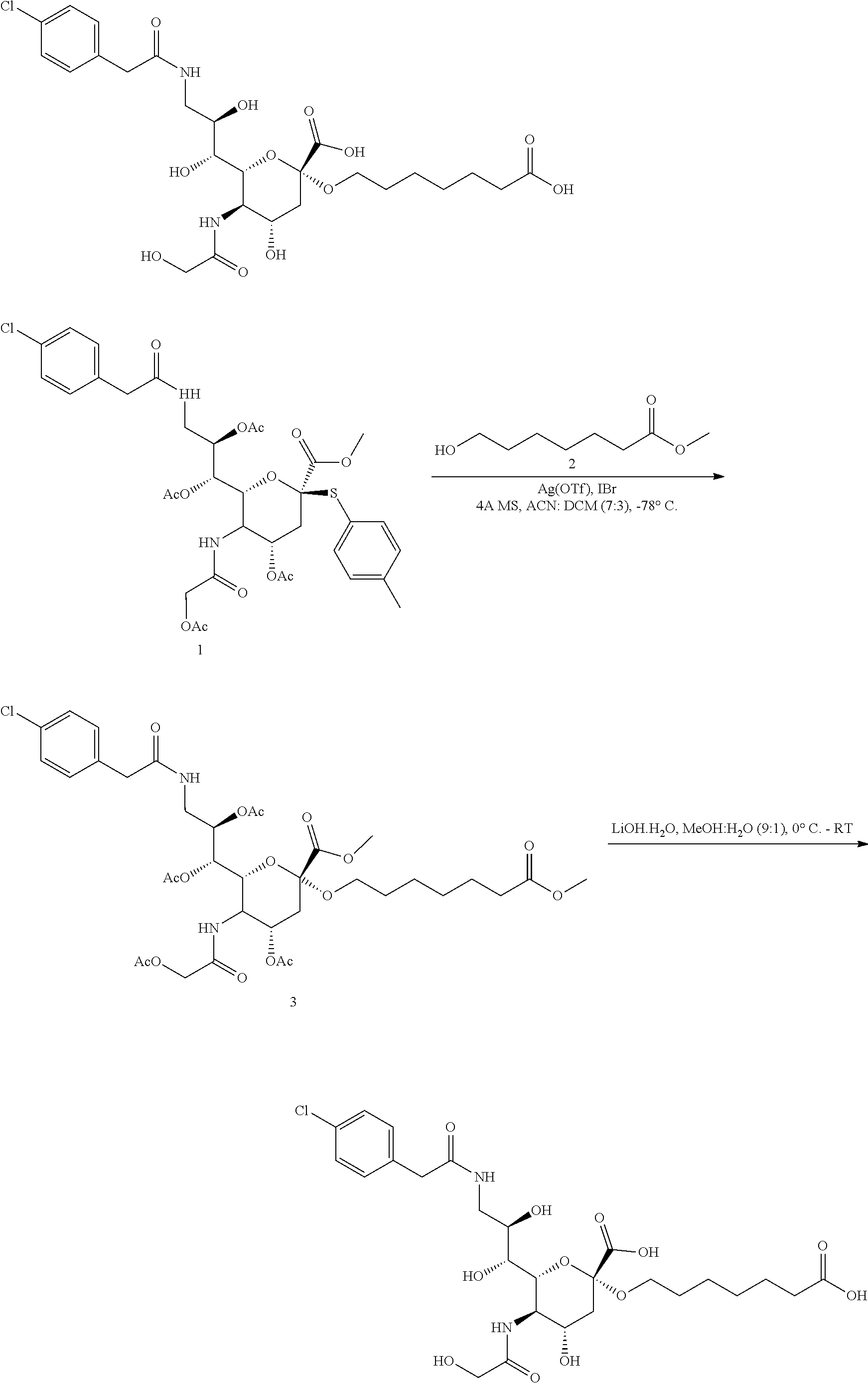

### Synthesis of (1R,2R)-1-((2R,3R,4S,6R)-4-acetoxy-3-(2-acetoxyacetamido)-6-((7-methoxy-7-oxoheptyl)oxy)-6-(methoxycarbonyl)tetrahydro-2H-pyran-2-yl)-3-(2-(4-chlorophenyl)acetamido)propane-1,2-diyl diacetate (3)

A mixture of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (1, 0.35 g, 0.46 mmol) and methyl 7-hydroxyheptanoate (2, 0.51 g, 3.20 mmol), silver(1+) trifluoromethanesulfonate (0.35 g, 1.37 mmol) and activated 4 Å powdered molecular sieves (1.00 g) in anhydrous dichloromethane (35 mL) and anhydrous acetonitrile (60 mL) was stirred at room temperature for 1 h. The solution was cooled to −78° C. and a solution of iodobromane (0.19 g, 0.91 mmol) in dichloromethane (5.0 mL) was added dropwise. The reaction mixture was stirred at the same temperature for 2 h. After completion, the reaction mixture was quenched by addition of triethylamine (1.0 mL) and warmed up to room temperature. The reaction mixture was filtered, and the filtrate was washed with aqueous saturated solution of sodium bicarbonate, dried, filtered, and concentrated. The crude residue obtained was purified by silica gel column chromatography using ethyl acetate in heptane as eluents to afford the desired product (1R,2R)-1-((2R,3R,4S,6R)-4-acetoxy-3-(2-acetoxyacetamido)-6-((7-methoxy-7-oxoheptyl)oxy)-6-(methoxycarbonyl)tetrahydro-2H-pyran-2-yl)-3-(2-(4-chlorophenyl)acetamido)propane-1,2-diyl diacetate (3). Yield: 0.20 g, 55%; LCMS m/z 801.05 [M+H]⁺.

### Synthesis of (2R,4S,5R,6R)-2-((6-carboxyhexyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Cpd. 98)

To a stirred solution of (1R,2R)-1-((2R,3R,4S,6R)-4-acetoxy-3-(2-acetoxyacetamido)-6-((7-methoxy-7-oxoheptyl)oxy)-6-(methoxycarbonyl)tetrahydro-2H-pyran-2-yl)-3-(2-(4-chlorophenyl)acetamido)propane-1,2-diyl diacetate (3, 0.25 g, 0.31 mmol) in methanol (5 mL), was added a solution of lithium hydroxide monohydrate (0.045 g, 1.87 mmol) in water (0.5 mL). The reaction mixture was stirred at room temperature for 6 h. After completion, the reaction mixture was treated with acidic resin (Dowex 50, H⁺) to pH ˜6 and the suspension was filtered. The filtrate was concentrated, and the crude residue obtained was purified by prep HPLC using acetonitrile in water (+0.1% TFA) as eluents to afford the desired product (2R,4S,5R,6R)-2-((6-carboxyhexyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Cpd. 98). Yield: 0.073 g, 52%; LCMS m/z 605.40 [M+H]+; ¹H NMR (400 MHz, Methanol-d₄): δ 7.31 (m, 4H), 4.03 (s, 2H), 3.92-3.71 (m, 5H), 3.65 (dd, J=14.4 Hz & 2.8 Hz, 1H), 3.55 (s, 2H), 3.44-3.38 (m, 2H), 3.33-3.23 (m, 1H), 2.73 (dd, J=12.4 Hz & 4.0 Hz, 1H), 2.30 (t, J=7.2 Hz, 2H), 1.74 (t, J=12.4 Hz, 1H), 1.63-1.55 (m, 4H), 1.42-1.37 (m, 4H).

### (2R,4S,5R,6R)-2-((7-carboxyheptyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Compound 97)

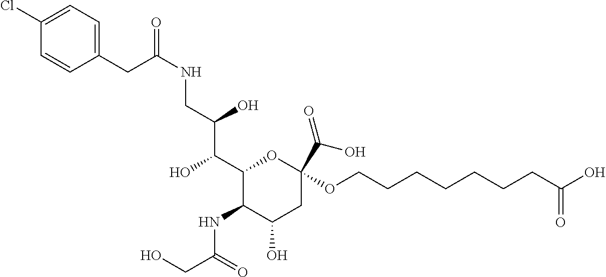 [SMILES: O=C(O)CCCCCCCO[C@]1(C(=O)O)C[C@H](O)[C@@H](NC(=O)CO)[C@H]([C@H](O)[C@H](O)CNC(=O)Cc2ccc(Cl)cc2)O1]

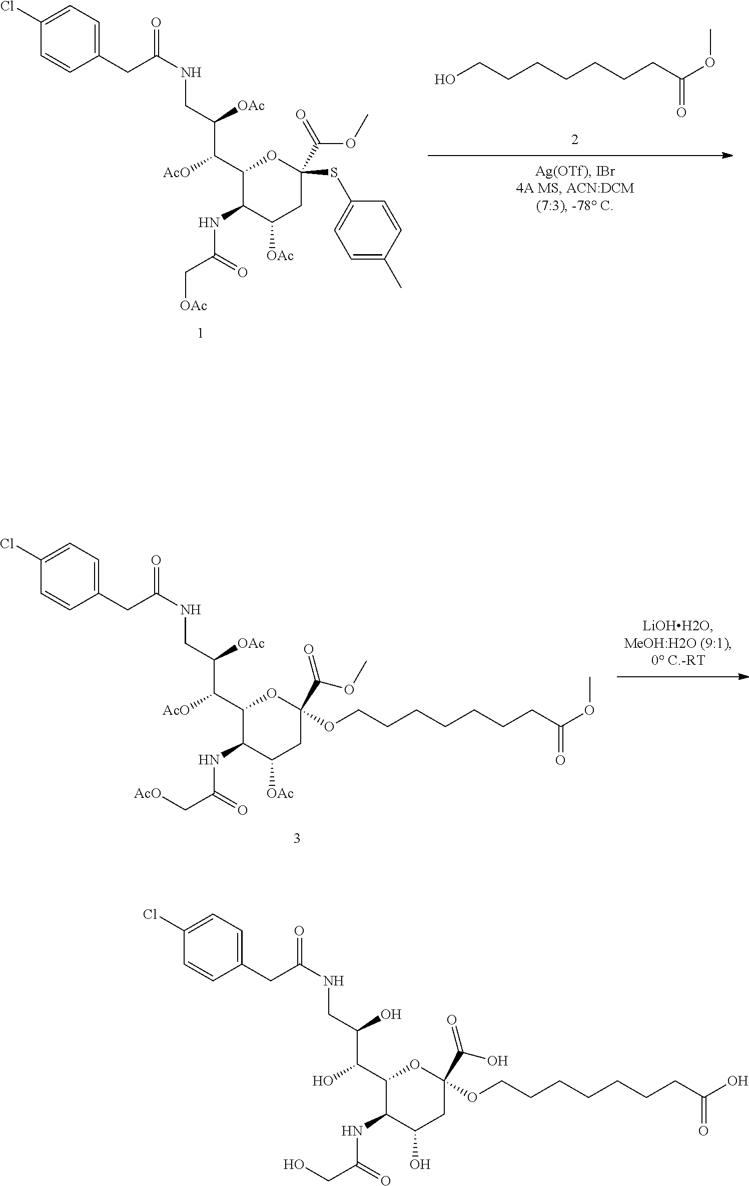 [SMILES: COC(=O)[C@]1(Sc2ccc(C)cc2)C[C@H](OC(C)=O)[C@@H](NC(=O)COC(C)=O)[C@H]([C@H](OC(C)=O)[C@@H](CNC(=O)Cc2ccc(Cl)cc2)OC(C)=O)O1 ; COC(=O)CCCCCCCO ; COC(=O)CCCCCCCO[C@]1(C(=O)OC)C[C@H](OC(C)=O)[C@@H](NC(=O)COC(C)=O)[C@H]([C@H](OC(C)=O)[C@@H](CNC(=O)Cc2ccc(Cl)cc2)OC(C)=O)O1 ; O=C(O)CCCCCCCO[C@]1(C(=O)O)C[C@H](O)[C@@H](NC(=O)CO)[C@H]([C@H](O)[C@H](O)CNC(=O)Cc2ccc(Cl)cc2)O1]

### Synthesis of (1R,2R)-1-((2R,3R,4S,6R)-4-acetoxy-3-(2-acetoxyacetamido)-6-((8-methoxy-8-oxooctyl)oxy)-6-(methoxycarbonyl)tetrahydro-2H-pyran-2-yl)-3-(2-(4-chlorophenyl)acetamido)propane-1,2-diyl diacetate (3)

A mixture of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (1, 0.40 g, 0.52 mmol) and methyl 8-hydroxyoctanoate (2, 0.45 g, 2.61 mmol), silver(1+) trifluoromethanesulfonate (0.40 g, 1.57 mmol) and activated 4 Å powdered molecular sieves (1.00 g) in anhydrous dichloromethane (35 mL) and anhydrous acetonitrile (60 mL) was stirred at room temperature for 1 h. The solution was cooled to −78° C. and a solution of iodobromane (0.21 g, 1.05 mmol) in dichloromethane (5.0 mL) was added dropwise. The reaction mixture was stirred at the same temperature for 2 h. After completion, the reaction mixture was quenched by addition of triethylamine (1.0 mL) and warmed up to room temperature. The reaction mixture was filtered, and the filtrate was washed with aqueous saturated solution of sodium bicarbonate and dried, filtered, and concentrated. The crude residue obtained was purified by silica gel column chromatography using ethyl acetate in heptane as eluents to afford the desired product (1R,2R)-1-((2R,3R,4S,6R)-4-acetoxy-3-(2-acetoxyacetamido)-6-((8-methoxy-8-oxooctyl)oxy)-6-(methoxycarbonyl)tetrahydro-2H-pyran-2-yl)-3-(2-(4-chlorophenyl)acetamido)propane-1,2-diyl diacetate (3). Yield: 0.38 g, 40%; LCMS m/z 815.10 [M+H]⁺.

### Synthesis of (2R,4S,5R,6R)-2-((7-carboxyheptyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Cpd. 97)

To a stirred solution of (1R,2R)-1-((2R,3R,4S,6R)-4-acetoxy-3-(2-acetoxyacetamido)-6-((8-methoxy-8-oxooctyl)oxy)-6-(methoxycarbonyl)tetrahydro-2H-pyran-2-yl)-3-(2-(4-chlorophenyl)acetamido)propane-1,2-diyl diacetate (3, 0.35 g, 0.43 mmol) in methanol (5 mL), was added a solution of lithium hydroxide monohydrate (0.072 g, 3.01 mmol) in water (0.5 mL). The reaction mixture was stirred at room temperature for 6 h. After completion, the reaction mixture was treated with acidic resin (Dowex 50, H⁺) to pH ˜6 and the suspension was filtered. The filtrate was concentrated, and the crude residue obtained was purified by prep HPLC using acetonitrile in water (+0.1% TFA) as eluents to afford the desired product (2R,4S,5R,6R)-2-((7-carboxyheptyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Cpd. 97). Yield: 0.099 g, 78%; LCMS m/z 619.30 [M+H]+; ¹H NMR (400 MHz, Methanol-d₄): δ 7.33-7.28 (m, 4H), 4.03 (s, 2H), 3.91-3.70 (m, 5H), 3.65 (dd, J=14.4 Hz & 2.8 Hz, 1H), 3.55 (s, 2H), 3.44-3.38 (m, 2H), 3.28-3.23 (m, 1H), 2.73 (dd, J=12.4 Hz & 4.0 Hz, 1H), 2.30 (t, J=7.2 Hz, 2H), 1.74 (t, J=12.4 Hz, 1H), 1.63-1.55 (m, 4H), 1.36 (brs, 6H).

### (2R,4S,5R,6R)-6-((1R,2R)-3-(2-(4-chlorophenyl)acetamido)-1,2-dihydroxypropyl)-2-((7-(hex-5-yn-1-ylamino)-7-oxoheptyl)oxy)-4-hydroxy-5-(2-hydroxyacetamido)tetrahydro-2H-pyran-2-carboxylic acid (Compound 96)

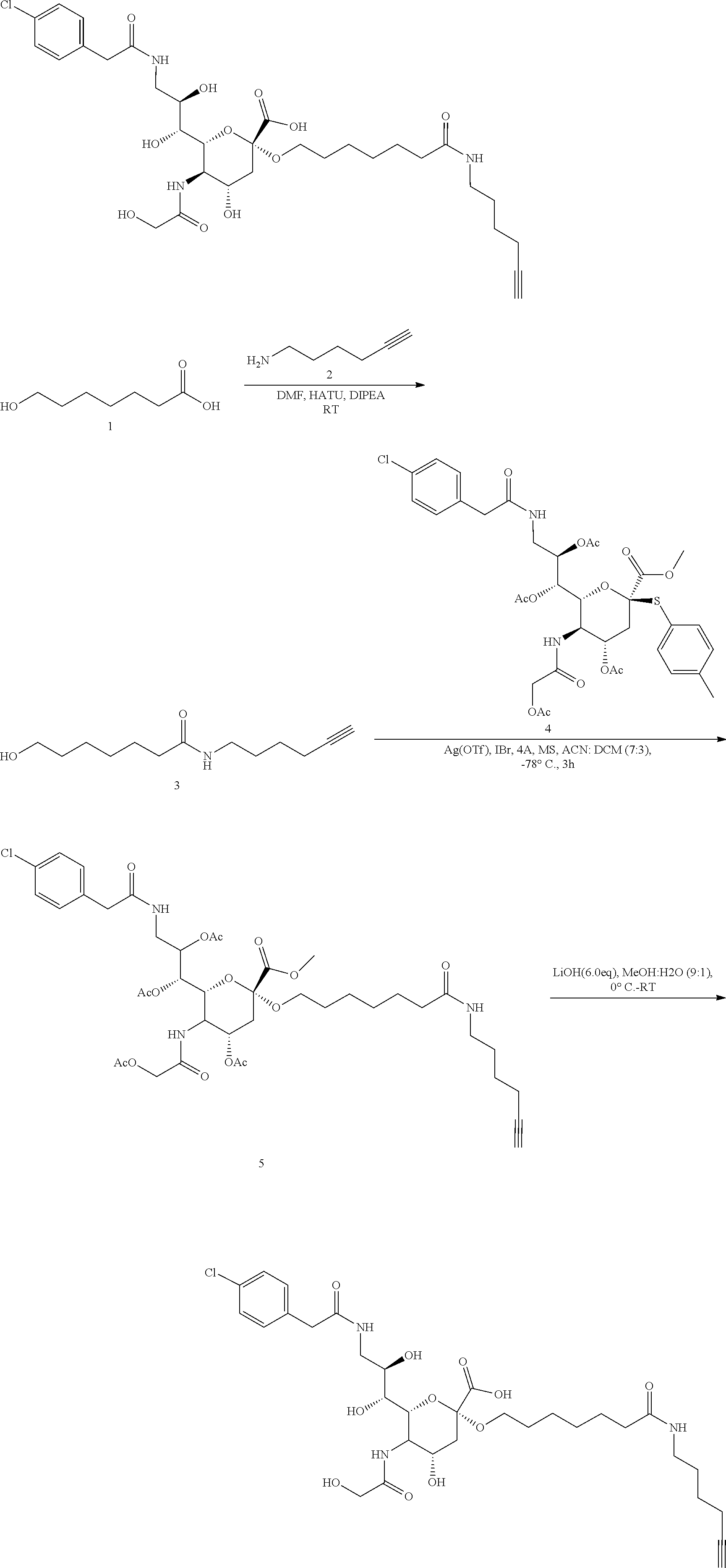

### Synthesis of N-(hex-5-yn-1-yl)-7-hydroxyheptanamide (3)

To a stirred solution of 7-hydroxyheptanoic acid (1, 0.80 g, 5.47 mmol) and hex-5-yn-1-amine (2, 0.80 g, 8.21 mmol) in dimethyl formamide (20.0 mL) were added 1-[Bis(dimethylamino)methylene]-1H-1,2,3-triazolo[4,5-b]pyridinium 3-oxide hexafluorophosphate (2.5 g, 6.47 mmol) and ethylbis(propan-2-yl)amine (3.03 mL, 16.40 mmol) sequentially at 0° C. then stirred at room temperature for 12 h. After completion, the reaction mixture was concentrated, and the crude residue obtained was purified by silica gel column chromatography using ethyl acetate in hexane as eluents to afford the desired product N-(hex-5-yn-1-yl)-7-hydroxyheptanamide (3). Yield: 0.80 g, 64% (over two steps); LCMS (ESI) m/z 226.20 [M+H]⁺.

### Synthesis of (1R,2R)-1-((2R,3R,4S,6R)-4-acetoxy-3-(2-acetoxyacetamido)-6-((7-(hex-5-yn-1-ylamino)-7-oxoheptyl)oxy)-6-(methoxycarbonyl)tetrahydro-2H-pyran-2-yl)-3-(2-(4-chlorophenyl)acetamido)propane-1,2-diyl diacetate (5)

A mixture of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (4, 0.70 g, 0.91 mmol) and N-(hex-5-yn-1-yl)-7-hydroxyheptanamide (3, 1.03 g, 4.57 mmol), silver(1+) trifluoromethanesulfonate (0.71 g, 2.74 mmol) and activated 4 Å powdered molecular sieves (1.00 g) in anhydrous dichloromethane (55 mL) and anhydrous acetonitrile (90 mL) was stirred at room temperature for 1 h. The solution was cooled to −78° C. and a solution of iodobromane (0.38 g, 1.83 mmol) in dichloromethane (5.0 mL) was added dropwise. The reaction mixture was stirred at the same temperature for 2 h. After completion, the reaction mixture was quenched by addition of triethylamine (2.0 mL) and warmed up to room temperature. The reaction mixture was filtered, and the filtrate was washed with aqueous saturated solution of sodium bicarbonate and dried, filtered, and concentrated. The crude residue obtained was purified by silica gel column chromatography using ethyl acetate in heptane as eluents to afford the desired product (1R,2R)-1-((2R,3R,4S,6R)-4-acetoxy-3-(2-acetoxyacetamido)-6-((7-(hex-5-yn-1-ylamino)-7-oxoheptyl)oxy)-6-(methoxycarbonyl)tetrahydro-2H-pyran-2-yl)-3-(2-(4-chlorophenyl)acetamido)propane-1,2-diyl diacetate (5). Yield: 0.51 g, 67%; LCMS m/z 866.15 [M+H]⁺.

### Synthesis of (2R,4S,5R,6R)-6-((1R,2R)-3-(2-(4-chlorophenyl)acetamido)-1,2-dihydroxypropyl)-2-((7-(hex-5-yn-1-ylamino)-7-oxoheptyl)oxy)-4-hydroxy-5-(2-hydroxyacetamido)tetrahydro-2H-pyran-2-carboxylic acid (Cpd. 96)

To a stirred solution of (1R,2R)-1-((2R,3R,4S,6R)-4-acetoxy-3-(2-acetoxyacetamido)-6-((7-(hex-5-yn-1-ylamino)-7-oxoheptyl)oxy)-6-(methoxycarbonyl)tetrahydro-2H-pyran-2-yl)-3-(2-(4-chlorophenyl)acetamido)propane-1,2-diyl diacetate (5, 0.40 g, 0.46 mmol) in methanol (5 mL) was added a solution of lithium hydroxide monohydrate (0.066 g, 2.77 mmol) in water (0.5 mL). The reaction mixture was stirred at room temperature for 6 h. After completion, the reaction mixture was treated with acidic resin (Dowex 50, H⁺) to pH˜6 and the suspension was filtered. The filtrate was concentrated, and the crude residue obtained was purified by prep HPLC using acetonitrile in water (+0.1% TFA) as eluents to afford the desired product (2R,4S,5R,6R)-6-((1R,2R)-3-(2-(4-chlorophenyl)acetamido)-1,2-dihydroxypropyl)-2-((7-(hex-5-yn-1-ylamino)-7-oxoheptyl)oxy)-4-hydroxy-5-(2-hydroxyacetamido)tetrahydro-2H-pyran-2-carboxylic acid (Cpd. 96). Yield: 0.036 g, 24%; LCMS m/z 684.50 [M+H]+; ¹H NMR (400 MHz, Methanol-d₄): δ 7.34-7.29 (m, 4H), 4.04 (s, 2H), 3.92-3.66 (m, 6H), 3.55 (s, 2H), 3.44-3.37 (m, 2H), 3.28-3.18 (m, 3H), 2.73 (dd, J=12.4 Hz & 4.0 Hz, 1H), 2.23-2.16 (m, 5H), 1.74 (t, J=12.8 Hz, 1H), 1.66-1.52 (m, 8H), 1.39-1.35 (m, 4H).

### (2R,4S,5R,6R)-6-((1R,2R)-3-(2-(4-chlorophenyl)acetamido)-1,2-dihydroxypropyl)-4-hydroxy-2-((5-((hydroxy(prop-2-yn-1-yloxy)phosphoryl)oxy)pentyl)oxy)-5-(2-hydroxyacetamido)tetrahydro-2H-pyran-2-carboxylic acid (Compound 95)

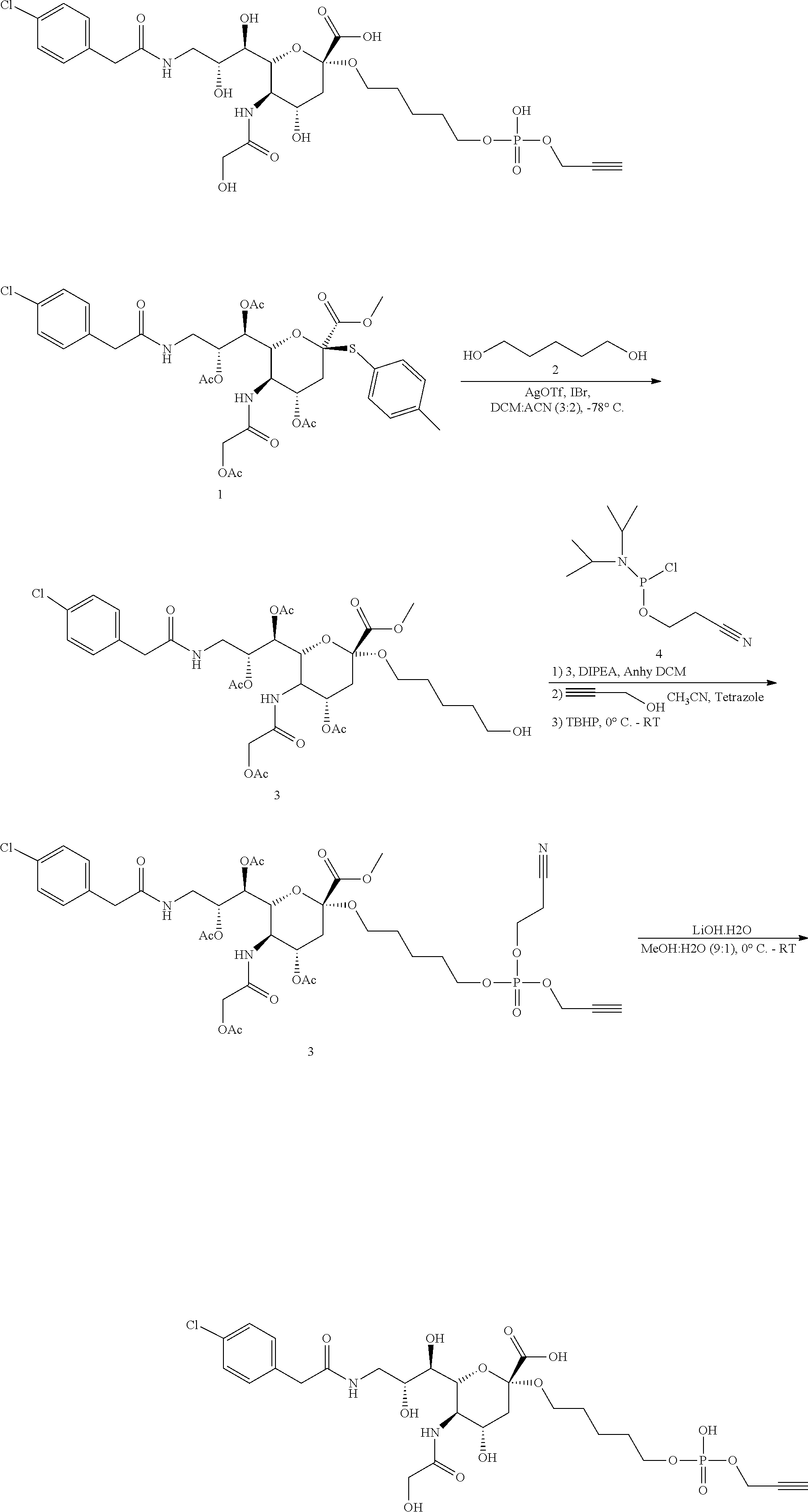

### Synthesis of (1R,2R)-1-((2R,3R,4S,6R)-4-acetoxy-3-(2-acetoxyacetamido)-6-((5-hydroxypentyl)oxy)-6-(methoxycarbonyl)tetrahydro-2H-pyran-2-yl)-3-(2-(4-chlorophenyl)acetamido)propane-1,2-diyl diacetate (3)

A mixture of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (1, 0.70 g, 0.91 mmol), pentane-1,5-diol (2, 0.67 g, 6.40 mmol), silver trifluoromethanesulfonate (0.70, 2.74 mmol) and activated 4 Å powdered molecular sieves (0.5 g) in anhydrous dichloromethane (75 mL) and acetonitrile (120 mL) was stirred at room temperature for 2 h. A solution of Iodine mono bromide (0.37 mg, 1.83 mmol) in dichloromethane (5 mL) was added at −78° C. then stirred at the same temperature for 2 h. After completion, the reaction mixture was quenched by addition of triethyl amine (0.1 mL) and warmed up to room temperature. The reaction mixture was filtered, and the filtrate was washed with a saturated solution of sodium thiosulphate and dried, filtered, and concentrated. The crude residue obtained was purified by silica gel column chromatography using ethyl acetate in hexane as eluents to afford the desired product (1R,2R)-1-((2R,3R,4S,6R)-4-acetoxy-3-(2-acetoxyacetamido)-6-((5-hydroxypentyl)oxy)-6-(methoxycarbonyl)tetrahydro-2H-pyran-2-yl)-3-(2-(4-chlorophenyl)acetamido)propane-1,2-diyl diacetate (3). Yield: 0.3 g, 44%; LCMS (ESI) m/z 745.10 [M+H]⁺.

### Synthesis of (1R,2R)-1-((2R,3R,4S,6R)-4-acetoxy-3-(2-acetoxyacetamido)-6-((5-(((2-cyanoethoxy)(prop-2-yn-1-yloxy)phosphoryl)oxy)pentyl)oxy)-6-(methoxycarbonyl)tetrahydro-2H-pyran-2-yl)-3-(2-(4-chlorophenyl)acetamido)propane-1,2-diyl diacetate (5)

To the stirred solution of 3-((chloro(diisopropylamino)phosphaneyl)oxy)propanenitrile (4, 0.09 g, 0.39 mmol) in dichloromethane (4 ml), ethylbis(propan-2-yl)amine (0.11 mL, 0.59 mmol) was added dropwise at 0° C. then stirred at the same temperature for 10 min, then (1R,2R)-1-((2R,3R,4S,6R)-4-acetoxy-3-(2-acetoxyacetamido)-6-((5-hydroxypentyl)oxy)-6-(methoxycarbonyl)tetrahydro-2H-pyran-2-yl)-3-(2-(4-chlorophenyl)acetamido)propane-1,2-diyl diacetate (3, 0.15 g, 0.19 mmol) in dichloromethane (1 mL) was added was stirred for another 30 min at room temperature. After completion, propargyl alcohol (0.022 mL, 0.39 mmol) and tetrazole (0.028 g, 0.39 mmol) in acetonitrile (0.5 mL) were added sequentially at 0° C. was stirred for another 1 h at room temperature. After completion TBHP (0.07 mL) was added at 0° C. then stirred at room temperature for 1 h. After completion, the reaction mixture was concentrated to afford the desired product (1R,2R)-1-((2R,3R,4S,6R)-4-acetoxy-3-(2-acetoxyacetamido)-6-((5-(((2-cyanoethoxy)(prop-2-yn-1-yloxy)phosphoryl)oxy)pentyl)oxy)-6-(methoxycarbonyl)tetrahydro-2H-pyran-2-yl)-3-(2-(4-chlorophenyl)acetamido)propane-1,2-diyl diacetate (5) as a crude which was used for the next step without further purification. Yield: 0.15 g, crude; LCMS m/z 917.0 [M+H]⁺.

### Synthesis of (2R,4S,5R,6R)-6-((1R,2R)-3-(2-(4-chlorophenyl)acetamido)-1,2-dihydroxypropyl)-4-hydroxy-2-((5-((hydroxy(prop-2-yn-1-yloxy)phosphoryl)oxy)pentyl)oxy)-5-(2-hydroxyacetamido)tetrahydro-2H-pyran-2-carboxylic acid (Cpd. 95)

To the stirred solution of (1R,2R)-1-((2R,3R,4S,6R)-4-acetoxy-3-(2-acetoxyacetamido)-6-((5-(((2-cyanoethoxy)(prop-2-yn-1-yloxy)phosphoryl)oxy)pentyl)oxy)-6-(methoxycarbonyl)tetrahydro-2H-pyran-2-yl)-3-(2-(4-chlorophenyl)acetamido)propane-1,2-diyl diacetate (5, 0.15 g, 0.17 mmol) in methanol (5.0 mL) and water (0.5 mL), lithium hydroxide monohydrate (0.023 g, 0.99 mmol) was added at 0° C. The reaction mixture was stirred at room temperature for 3 h. After completion, the reaction mixture was quenched with acidic resin (Dowex-H+) until pH˜6. to pH˜6 and filtered. The filtrate was concentrated. The crude residue obtained was purified by prep-HPLC using acetonitrile in water (+0.1% TFA) as to afford the desired product (2R,4S,5R,6R)-6-((1R,2R)-3-(2-(4-chlorophenyl)acetamido)-1,2-dihydroxypropyl)-4-hydroxy-2-((5-((hydroxy(prop-2-yn-1-yloxy)phosphoryl)oxy)pentyl)oxy)-5-(2-hydroxyacetamido)tetrahydro-2H-pyran-2-carboxylic acid (Cpd. 95); Yield: 0.014 g, 10%; LCMS m/z 695.4 [M+H]+; ¹H-NMR (400 MHz, Methanol-d4): δ 7.90 (d, J=8.0 Hz, 1H), 7.32-7.27 (m, 4H), 4.60 (dd, J=10.0, 3.0 Hz, 2H), 4.03-3.99 (m, 4H), 3.90-3.75 (m, 4H), 3.71-3.69 (m, 1H), 3.65 (dd, J=14.0 Hz & 3.2 Hz, 1H), 3.56 (s, 2H), 3.48-3.40 (m, 1H), 3.38-3.30 (m, 2H), 3.0 (t, J=3.0 Hz, 1H), 2.71 (dd, J=10.8 Hz & 4.0 Hz, 1H), 1.76-1.66 (m, 3H), 1.60-1.56 (m, 2H), 1.50-1.44 (m, 2H), 1.33-1.26 (m, 2H).

### (2R,4S,5R,6R)-6-((1R,2R)-1,2-dihydroxy-3-(2-(4-(4-hydroxypiperidin-1-yl)phenyl)acetamido)propyl)-4-hydroxy-5-(2-hydroxyacetamido)-2-((6-(prop-2-yn-1-yloxy)hexyl)oxy)tetrahydro-2H-pyran-2-carboxylic acid (Compound 194)

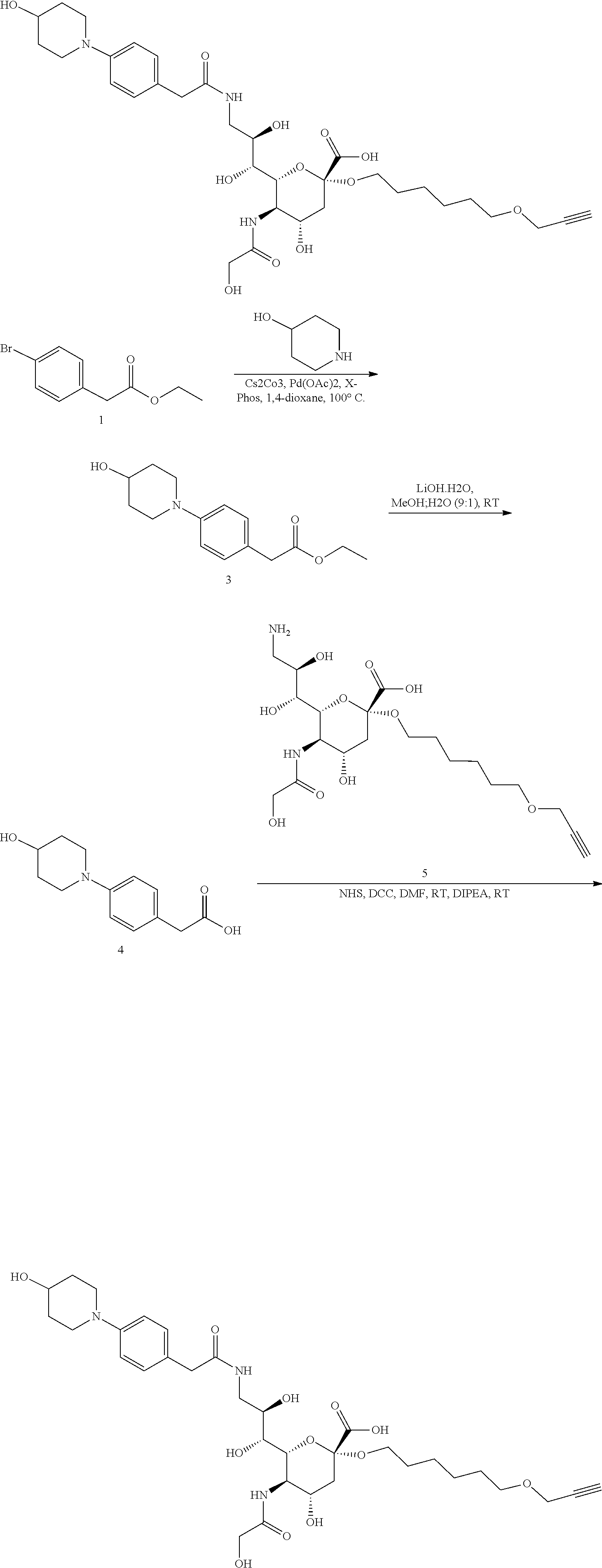

### Synthesis of ethyl 2-(4-(4-hydroxypiperidin-1-yl)phenyl)acetate (3)

To a stirred solution of ethyl 2-(4-bromophenyl)acetate (1, 0.50 g, 2.06 mmol), piperidin-4-ol (0.250 g, 2.47 mmol) and cesium carbonate (1.01 g, 2.06 mmol) in 1,4-dioxane (10 mL). nitrogen gas was purged in reaction mixture for 10 min, then dicyclohexyl[2′,4′,6′-tris(isopropyl)-2-biphenylyl]phosphine (0.196 g, 0.411 mmol) and palladium bis(acetate) (0.23 g, 0.103 mmol) was added was stirred at 100° C. for 12 h. After completion, the reaction mixture was dissolved in ethyl acetate and the resulting solution was washed with water. The organic layer was separated, dried and concentrated. The crude residue obtained was purified by silica gel column chromatography using in ethyl acetate in heptane as eluents to afford the desired product ethyl 2-(4-(4-hydroxypiperidin-1-yl)phenyl)acetate (3). Yield: 0.30 g, 55%; LCMS (ESI) m/z 264.10 [M+H]⁺.

### Synthesis of 2-(4-(4-hydroxypiperidin-1-yl)phenyl)acetic acid (4)

To a stirred solution of ethyl 2-(4-(4-hydroxypiperidin-1-yl)phenyl)acetate (3, 0.30 g, 1.14 mmol) in methanol (4.0 mL) and water (1.0 mL) was added lithium hydroxide monohydrate (0.098 g, 2.28 mmol) at 0° C. The reaction mixture was stirred at room temperature for 6 h. After completion, the reaction mixture was treated with acidic resin (Dowex 50, H⁺) to pH˜6 and the suspension was filtered. The filtrate was concentrated, and the crude residue obtained was purified by trituration with diethyl ether to afford the desired product 2-(4-(4-hydroxypiperidin-1-yl)phenyl)acetic acid (4). Yield: 0.250 g, 93%; LCMS m/z 236.10 [M+H]⁺.

### Synthesis of (2R,4S,5R,6R)-6-((1R,2R)-1,2-dihydroxy-3-(2-(4-(4-hydroxypiperidin-1-yl)phenyl)acetamido)propyl)-4-hydroxy-5-(2-hydroxyacetamido)-2-((6-(prop-2-yn-1-yloxy)hexyl)oxy)tetrahydro-2H-pyran-2-carboxylic acid (Cpd. 194)

To a stirred solution of 2-(4-(4-hydroxypiperidin-1-yl)phenyl)acetic acid (4, 0.100 g, 0.425 mmol) in dimethylformamide (3.0 mL) N,N′-Dicyclohexylcarbodiimide (0.087 g, 0.425 mmol) and N-hydroxysuccinimide (0.048 g, 0.425 mmol) was added at 0° C. and stirred the reaction mixture for 3 h. (2R,4S,5R,6R)-6-((1R,2R)-3-amino-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (5, 0.196 g, 0.425 mmol) and ethylbis(propan-2-yl)amine (0.37 mL, 2.15 mmol) was added at 0° C. then stirred at room temperature for 4 h. After completion, the reaction mixture was concentrated, and the crude residue obtained was purified by prep HPLC using acetonitrile in water (+0.1% TFA) as eluents to afford the desired product (2R,4S,5R,6R)-6-((1R,2R)-1,2-dihydroxy-3-(2-(4-(4-hydroxypiperidin-1-yl)phenyl)acetamido)propyl)-4-hydroxy-5-(2-hydroxyacetamido)-2-((6-(prop-2-yn-1-yloxy)hexyl)oxy)tetrahydro-2H-pyran-2-carboxylic acid (Cpd. 194). Yield: 0.015 g, 6%; LCMS m/z 680.50 [M+H]+. 1H NMR (400 MHz, methanol-d4): δ 7.30 (d, J=8.8 Hz, 2H), 7.19 (d, J=8.4 Hz, 2H), 4.12 (d, J=2.4 Hz, 2H), 4.01 (s, 2H), 3.90-3.75 (m, 5H), 3.73-3.60 (m, 4H), 3.53-3.49 (m, 4H), 3.44-3.34 (m, 2H), 3.25-3.24 (m, 1H), 3.15-3.10 (m, 2H), 2.81 (t, J=2.4 Hz, 1H), 2.73 (dd, J=12.4 Hz & 4.0 Hz, 1H), 2.07-2.03 (m, 1H), 1.82-1.67 (m, 3H), 1.57-1.55 (m, 4H), 1.38-1.37 (m, 4H).

### (2R,4S,5R,6R)-6-((1R,2R)-3-(2-(4-cyclopropylphenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Compound 161)

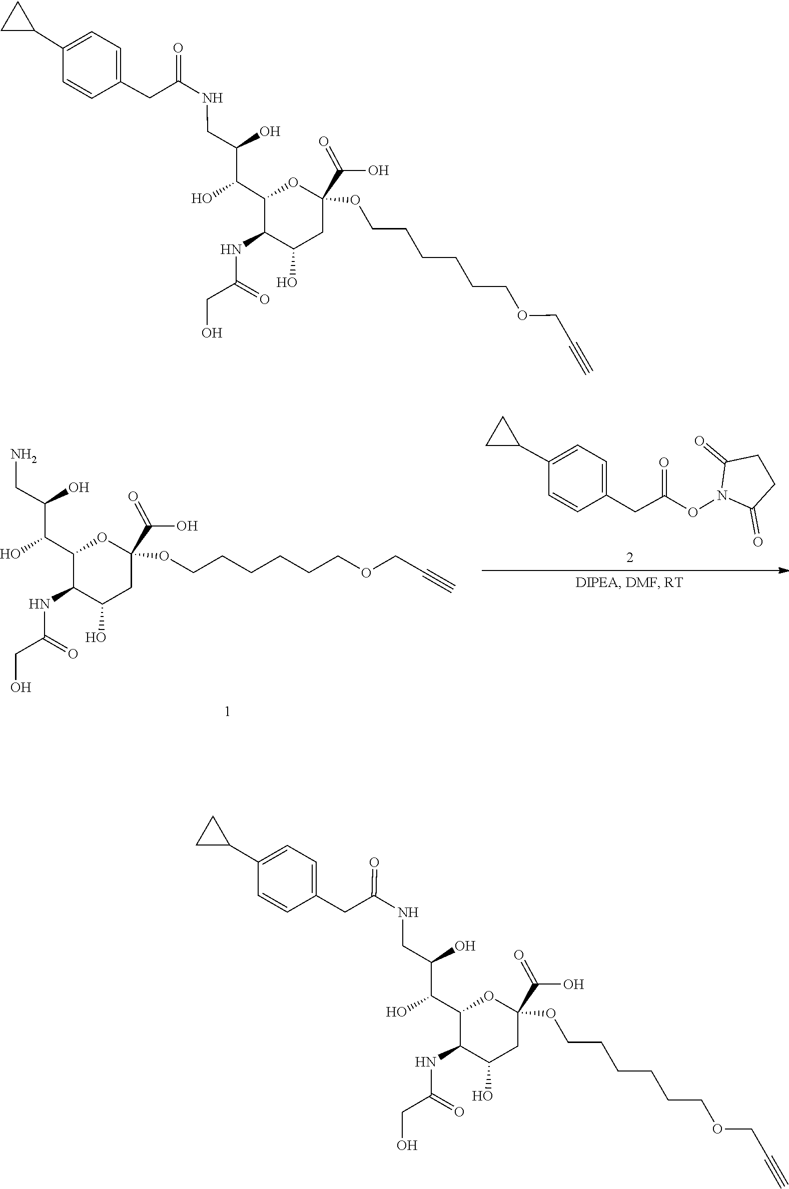 [SMILES: C#CCOCCCCCCO[C@]1(C(=O)O)C[C@H](O)[C@@H](NC(=O)CO)[C@H]([C@H](O)[C@H](O)CNC(=O)Cc2ccc(C3CC3)cc2)O1 ; C#CCOCCCCCCO[C@]1(C(=O)O)C[C@H](O)[C@@H](NC(=O)CO)[C@H]([C@H](O)[C@H](O)CN)O1 ; O=C(Cc1ccc(C2CC2)cc1)ON1C(=O)CCC1=O ; C#CCOCCCCCCO[C@]1(C(=O)O)C[C@H](O)[C@@H](NC(=O)CO)[C@H]([C@H](O)[C@H](O)CNC(=O)Cc2ccc(C3CC3)cc2)O1]

### Synthesis of (2R,4S,5R,6R)-6-((1R,2R)-3-(2-(4-cyclopropylphenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Cpd. 161)

To a stirred solution of (2R,4S,5R,6R)-6-((1R,2R)-3-amino-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (1, 0.100 g, 0.216 mmol) in N,N-dimethylformamide (2.0 mL), N,N-diisopropylethylamine (0.188 mL, 1.08 mmol) and 2,5-dioxopyrrolidin-1-yl 2-(4-cyclopropylphenyl)acetate (2, 0.059 g, 0.216 mmol) was added at 0° C. then stirred at room temperature for 4 h. After completion, the reaction mixture was concentrated, and the crude residue obtained was purified by prep HPLC using acetonitrile in water (+0.1% TFA) as eluents to afford the desired product (2R,4S,5R,6R)-6-((1R,2R)-3-(2-(4-cyclopropylphenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Cpd. 161). Yield: 0.073 g, 54%; LCMS (ESI) m/z 621.50 [M+H]+. ¹H NMR (400 MHz, MeOD-d4): δ 7.89 (d, J=8.0 Hz, 1H), 7.16 (d, J=8.0 Hz, 2H), 7.01 (d, J=8.4 Hz, 2H), 4.12 (d, J=2.4 Hz, 2H), 4.01 (s, 2H), 3.86-3.74 (m, 4H), 3.70-3.68 (m, 1H), 3.63 (dd, J=14.0 Hz & 3.2 Hz, 1H), 3.52-3.48 (m, 4H), 3.40-3.37 (m, 2H), 3.26-3.20 (m, 1H), 2.81 (t, J=2.4 Hz, 1H), 2.71 (dd, J=12.4 Hz & 3.6 Hz, 1H), 1.90-1.84 (m, 1H), 1.72 (t, J=12.0 Hz, 1H), 1.57-1.55 (m, 4H), 1.38-1.36 (m, 4H), 0.95-0.90 (m, 2H), 0.66-0.62 (m, 2H).

### (2S,4R,5S,6S)-6-((1S,2S)-3-(2-(4-cyclopropylphenyl)acetamido)-1,2-dihydroxypropyl)-4-hydroxy-5-(2-hydroxyacetamido)-2-((6-((1-(15-oxo-15-(perfluorophenoxy)-3,6,9,12-tetraoxapentadecyl)-1H-1,2,3-triazol-4-yl)methoxy)hexyl)oxy)tetrahydro-2H-pyran-2-carboxylic acid (Compound 161P)

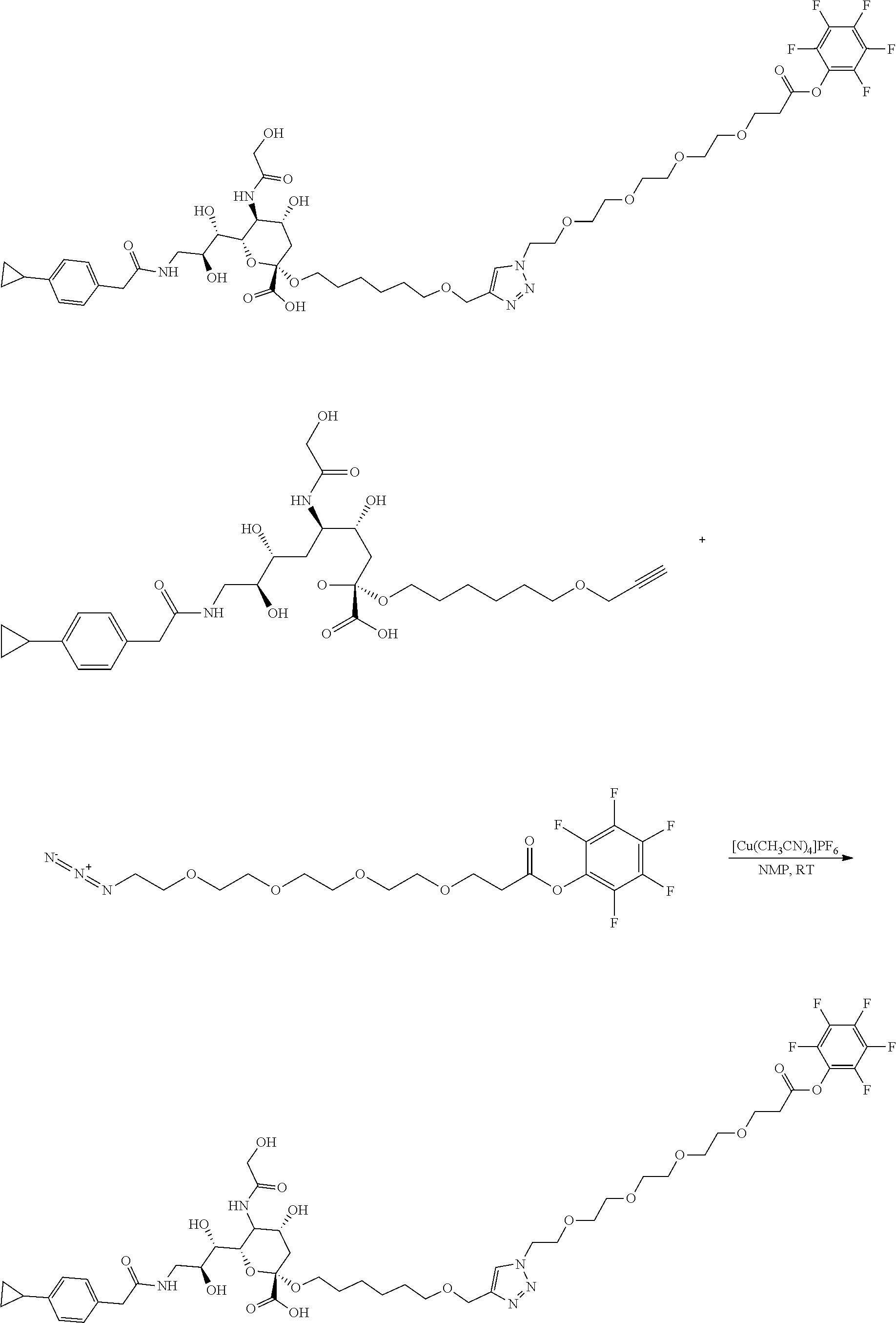

### Synthesis of (2S,4R,5S,6S)-6-((1S,2S)-3-(2-(4-cyclopropylphenyl)acetamido)-1,2-dihydroxypropyl)-4-hydroxy-5-(2-hydroxyacetamido)-2-((6-((1-(15-oxo-15-(perfluorophenoxy)-3,6,9,12-tetraoxapentadecyl)-1H-1,2,3-triazol-4-yl)methoxy)hexyl)oxy)tetrahydro-2H-pyran-2-carboxylic acid (161P)

To a solution of (2S,4R,5S,6S)-6-((1S,2S)-3-(2-(4-cyclopropylphenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (161, 50.6 mg, 81.5 umol) in anhydrous NMP (1.09 mL) was added Azido-PEG4-PFP-ester (41.0 mg, 89.7 umol), followed by Tetrakis(acetonitrile)copper(l) hexafluorophosphate (76.0 mg, 204 umol). The vial was sealed, and the mixture was stirred for 1 hour at room temperature. The mixture was filtered through a 0.45 μm PTFE filter. A few drops of acetic acid were added to the filtrate. The filtrate was purified by preparatory HPLC using acetonitrile and water (+0.05% TFA) as eluents. The fractions containing the desired product were lyophilized to afford 161P as a white solid. Yield: 59.3 mg, 67%; LCMS (ESI) m/z 1078 [M+H]⁺. ¹H NMR (500 MHz, DMSO-d₆): 8.02 (s, 1H), 7.88 (t, J=5.5 Hz, 1H), 7.84 (d, J=8.0 Hz, 1H), 7.10 (d, J=8.0 Hz, 2H), 6.96 (d, J=8.0 Hz, 2H), 5.51 (s, br, 1H), 4.96 (s, br, 1H), 4.70 (s, br, 1H), 4.49 (t, J=5.5 Hz, 2H), 4.46 (s, 2H), 4.28 (s, br, 1H), 3.86 (m, 2H), 3.82-3.78 (m, 1H), 3.79 (t, J=5.5 Hz, 2H), 3.75 (t, J=5.5 Hz, 2H), 3.67-3.58 (m, 3H), 3.55-3.45 (m, 18H), 3.40 (t, J=6.5 Hz, 2H), 3.20 (d, J=8.5 Hz, 1H), 3.01 (t, J=6.0 Hz, 2H), 2.95-2.90 (m, 1H), 1.87-1.82 (m, 1H), 1.53-1.43 (m, 5H), 1.26-1.25 (m, 4H), 0.91-0.87 (m, 2H), 0.62-0.58 (m, 2H).

### (2S,4R,5S,6S)-6-((1S,2S)-3-(2-(4-cyclopropylphenyl)acetamido)-1,2-dihydroxypropyl)-2-((6-((1-(18-(2,5-dioxo-2,5-dihydro-1H-pyrrol-1-yl)-15-oxo-3,6,9,12-tetraoxa-16-azaoctadecyl)-1H-1,2,3-triazol-4-yl)methoxy)hexyl)oxy)-4-hydroxy-5-(2-hydroxyacetamido)tetrahydro-2H-pyran-2-carboxylic acid (Compound 161M)

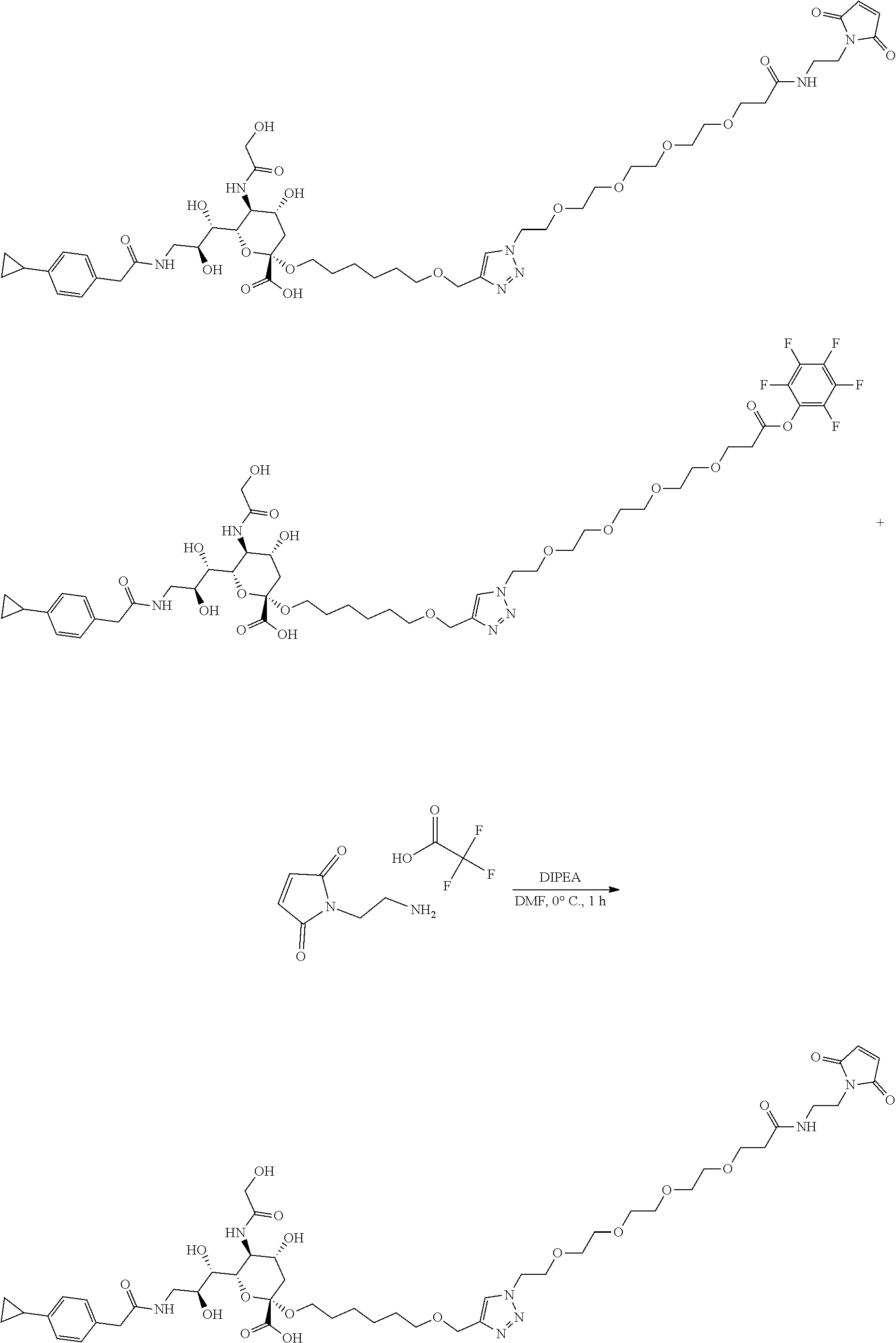 [SMILES: O=C(CCOCCOCCOCCOCCn1cc(COCCCCCCO[C@@]2(C(=O)O)C[C@@H](O)[C@H](NC(=O)CO)[C@@H]([C@@H](O)[C@@H](O)CNC(=O)Cc3ccc(C4CC4)cc3)O2)nn1)NCCN1C(=O)C=CC1=O ; O=C(Cc1ccc(C2CC2)cc1)NC[C@H](O)[C@H](O)[C@H]1O[C@](OCCCCCCOCc2cn(CCOCCOCCOCCOCCC(=O)Oc3c(F)c(F)c(F)c(F)c3F)nn2)(C(=O)O)C[C@@H](O)[C@@H]1NC(=O)CO ; NCCN1C(=O)C=CC1=O ; O=C(O)C(F)(F)F ; O=C(CCOCCOCCOCCOCCn1cc(COCCCCCCO[C@@]2(C(=O)O)C[C@@H](O)[C@H](NC(=O)CO)[C@@H]([C@@H](O)[C@@H](O)CNC(=O)Cc3ccc(C4CC4)cc3)O2)nn1)NCCN1C(=O)C=CC1=O]

### Synthesis of (2S,4R,5S,6S)-6-((1S,2S)-3-(2-(4-cyclopropylphenyl)acetamido)-1,2-dihydroxypropyl)-2-((6-((1-(18-(2,5-dioxo-2,5-dihydro-1H-pyrrol-1-yl)-15-oxo-3,6,9,12-tetraoxa-16-azaoctadecyl)-1H-1,2,3-triazol-4-yl)methoxy)hexyl)oxy)-4-hydroxy-5-(2-hydroxyacetamido)tetrahydro-2H-pyran-2-carboxylic acid (161M)

To a solution of (2S,4R,5S,6S)-6-((1S,2S)-3-(2-(4-cyclopropylphenyl)acetamido)-1,2-dihydroxypropyl)-4-hydroxy-5-(2-hydroxyacetamido)-2-((6-((1-(15-oxo-15-(perfluorophenoxy)-3,6,9,12-tetraoxapentadecyl)-1H-1,2,3-triazol-4-yl)methoxy)hexyl)oxy)tetrahydro-2H-pyran-2-carboxylic acid (161P, 41.0 mg, 38.0 umol) in DMF (951 uL) was added n-(2-aminoethyl)maleimide trifluoroacetate salt (10.1 mg, 39.9 umol). The mixture was cooled down to 0° C. and stirred for 10 min. DIEA (19.9 uL, 114 umol) was added and the mixture was stirred for 1 hr at 0° C. The reaction mixture was filtered and purified by preparatory HPLC using acetonitrile and water (+0.05% TFA) as eluents. The fractions containing the desired product were combined and lyophilized to afford 161M as a white solid. Yield: 27.4 mg, 70%; LCMS (ESI) m/z 1034 [M+H]⁺. ¹H NMR (500 MHz, DMSO-d₆): 8.03 (s, 1H), 7.93 (t, J=6.0 Hz, 1H), 7.88 (t, J=5.5 Hz, 1H), 7.84 (d, J=8.0 Hz, 1H), 7.11 (d, J=8.0 Hz, 2H), 6.99 (s, 2H), 6.96 (d, J=8.0 Hz, 2H), 4.50 (t, J=5.5 Hz, 2H), 4.46 (s, 2H), 3.87 (m, 2H), 3.82-3.78 (m, 1H), 3.80 (t, J=5.5 Hz, 2H), 3.67-3.59 (m, 3H), 3.57-3.50 (m, 8H), 3.48-3.40 (m, 14H), 3.29-3.26 (m, 2H), 3.21-3.16 (m, 3H), 2.95-2.90 (m, 1H), 2.21 (t, J=7.0 Hz, 2H), 1.88-1.82 (m, 1H), 1.54-1.43 (m, 5H), 1.26-1.25 (m, 4H), 0.91-0.88 (m, 2H), 0.62-0.59 (m, 2H).

### (2R,4S,5R,6R)-6-((1R,2R)-3-(2-(4-(tert-butyl)phenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Compound 160)

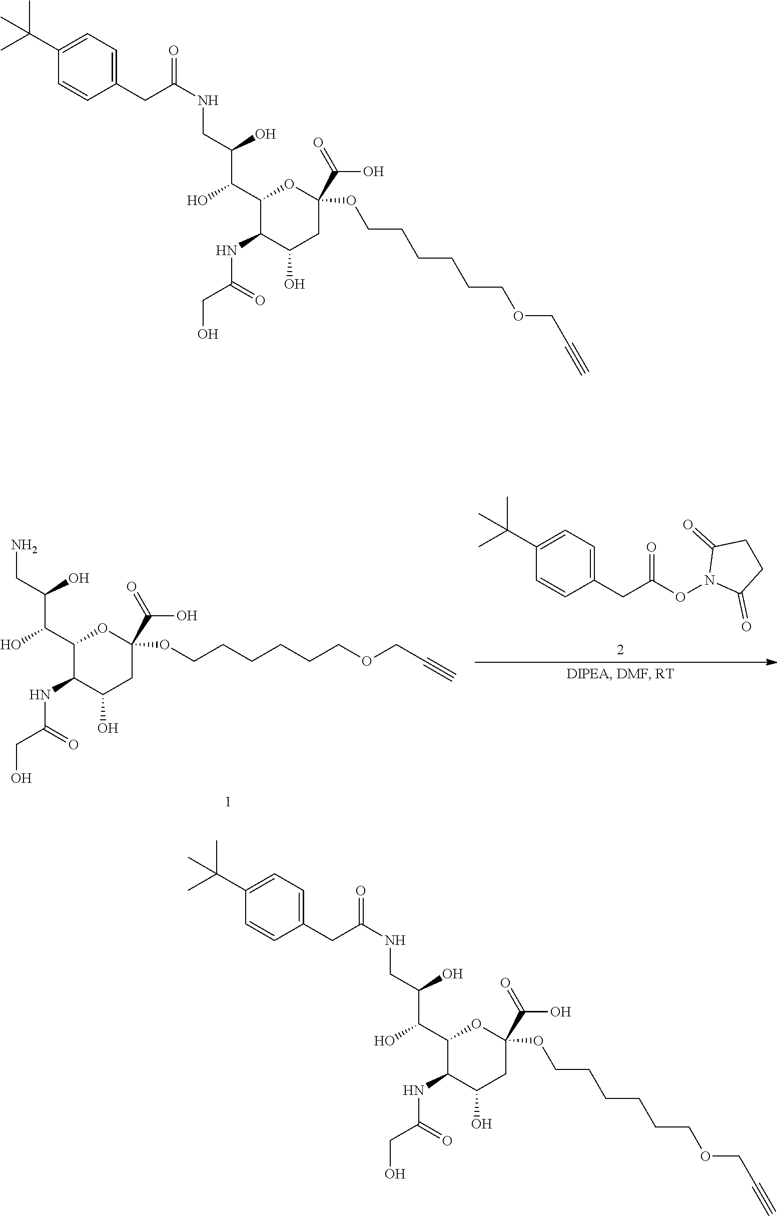

### Synthesis of (2R,4S,5R,6R)-6-((1R,2R)-3-(2-(4-(tert-butyl)phenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Cpd. 160)

To a stirred solution of (2R,4S,5R,6R)-6-((1R,2R)-3-amino-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (1, 0.100 g, 0.216 mmol) in N,N-dimethylformamide (2.0 mL) were added N,N-diisopropylethylamine (0.188 mL, 1.08 mmol) and 2,5-dioxopyrrolidin-1-yl 2-(4-(tert-butyl)phenyl)acetate (2, 0.062 g, 0.216 mmol) at 0° C. then stirred at room temperature for 4 h. After completion, the reaction mixture was concentrated, and the crude residue obtained was purified by prep HPLC using acetonitrile in water (+0.1% TFA) as eluents to afford the desired product (2R,4S,5R,6R)-6-((1R,2R)-3-(2-(4-(tert-butyl)phenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Cpd. 160). Yield: 0.061 g, 44%; LCMS (ESI) m/z 637.50 [M+H]+. ¹H NMR (400 MHz, MeOD-d4): δ 7.34 (d, J=8.4 Hz, 2H), 7.21 (d, J=8.0 Hz, 2H), 4.11 (d, J=2.4 Hz, 2H), 4.01 (s, 2H), 3.89-3.85 (m, 1H), 3.83-3.75 (m, 3H), 3.74-3.68 (m, 1H), 3.64 (dd, J=14.0 Hz & 3.2 Hz, 1H), 3.52-3.49 (m, 4H), 3.42-3.36 (m, 2H), 3.26-3.20 (m, 1H), 2.81 (t, J=2.4 Hz, 1H), 2.72 (dd, J=12.4 Hz & 4.0 Hz, 1H), 1.71 (t, J=12.0 Hz, 1H), 1.57-1.55 (m, 4H), 1.38-1.36 (m, 4H), 1.30 (s, 9H).

### (2R,4S,5R,6R)-6-((1R,2R)-1,2-dihydroxy-3-(2-(4-isopropylphenyl)acetamido)propyl)-4-hydroxy-5-(2-hydroxyacetamido)-2-((6-(prop-2-yn-1-yloxy)hexyl)oxy)tetrahydro-2H-pyran-2-carboxylic acid (Compound 159)

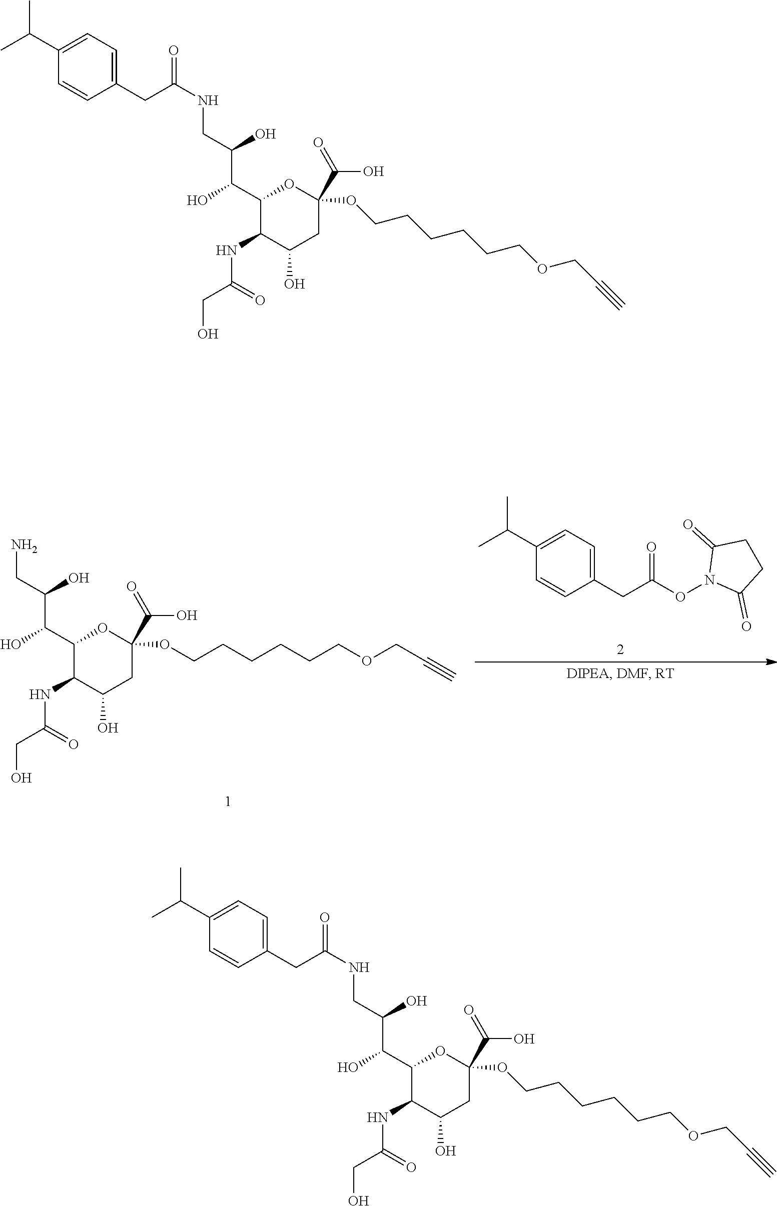

### Synthesis of (2R,4S,5R,6R)-6-((1R,2R)-1,2-dihydroxy-3-(2-(4-isopropylphenyl)acetamido)propyl)-4-hydroxy-5-(2-hydroxyacetamido)-2-((6-(prop-2-yn-1-yloxy)hexyl)oxy)tetrahydro-2H-pyran-2-carboxylic acid (Cpd. 159)

To a stirred solution of (2R,4S,5R,6R)-6-((1R,2R)-3-amino-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (1, 0.100 g, 0.216 mmol) in N,N-dimethylformamide (2.0 mL) were added N,N-diisopropylethylamine (0.188 mL, 1.08 mmol) and 2,5-dioxopyrrolidin-1-yl 2-(4-(tert-butyl)phenyl)acetate (2, 0.059 g, 0.216 mmol) at 0° C. then stirred at room temperature for 4 h. After completion, the reaction mixture was concentrated, and the crude residue obtained was purified by prep HPLC using acetonitrile in water (+0.1% TFA) as eluents to afford the desired product (2R,4S,5R,6R)-6-((1R,2R)-1,2-dihydroxy-3-(2-(4-isopropylphenyl)acetamido)propyl)-4-hydroxy-5-(2-hydroxyacetamido)-2-((6-(prop-2-yn-1-yloxy)hexyl)oxy)tetrahydro-2H-pyran-2-carboxylic acid (Cpd. 159). Yield: 0.040 g, 30%; LCMS (ESI) m/z 623.50 [M+H]+. ¹H NMR (400 MHz, MeOD-d4): δ 7.22-7.16 (m, 4H), 4.12 (d, J=2.4 Hz, 2H), 4.01 (s, 2H), 3.88-3.75 (m, 4H), 3.70-3.68 (m, 1H), 3.66-3.61 (m, 1H), 3.52-3.49 (m, 4H), 3.41-3.36 (m, 2H), 3.30-3.24 (m, 1H), 2.88-2.85 (m, 1H), 2.81 (t, J=2.4 Hz, 1H), 2.71 (dd, J=12.4 Hz & 3.6 Hz, 1H), 1.72 (t, J=12.0 Hz, 1H), 1.57-1.53 (m, 4H), 1.40-1.36 (m, 4H), 1.22 (d, J=6.8 Hz, 6H).

### (2R,4S,5R,6R)-6-((1R,2R)-1,2-dihydroxy-3-(2-(4-(trifluoromethyl)phenyl)acetamido)propyl)-4-hydroxy-5-(2-hydroxyacetamido)-2-((6-(prop-2-yn-1-yloxy)hexyl)oxy)tetrahydro-2H-pyran-2-carboxylic acid (Compound 158)

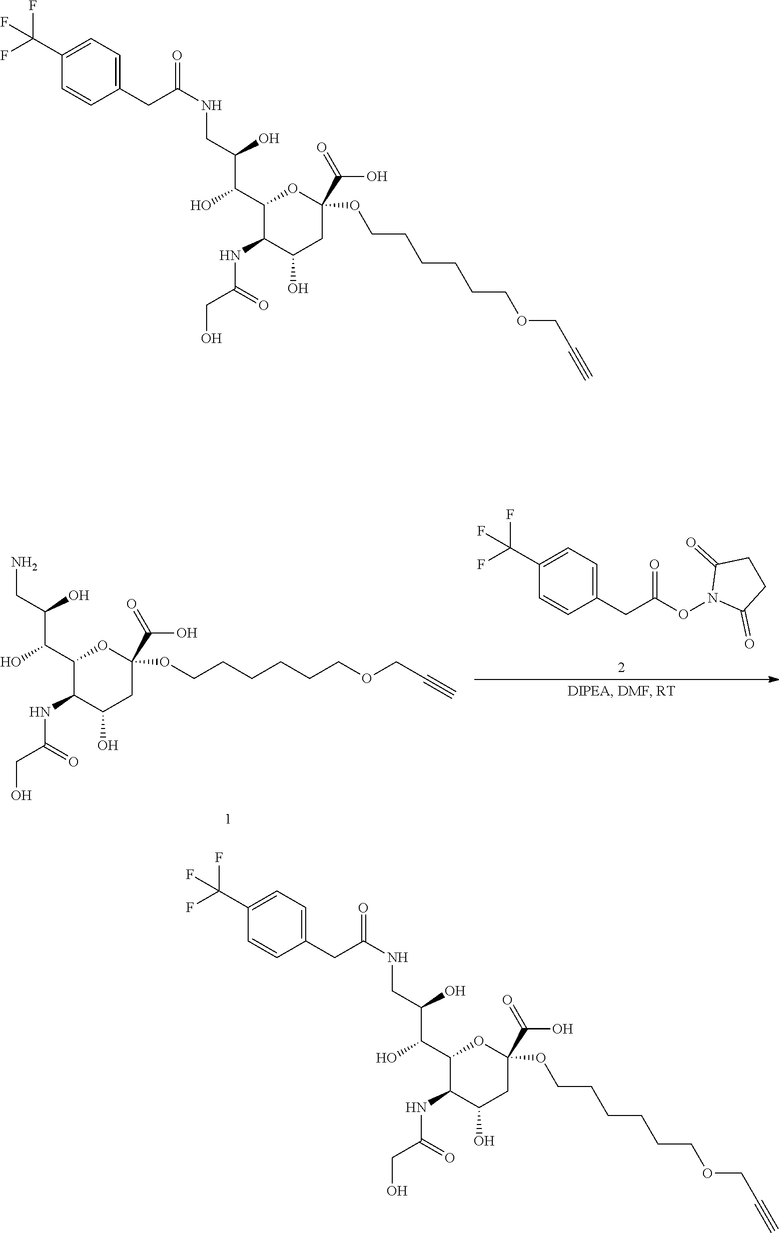 [SMILES: C#CCOCCCCCCO[C@]1(C(=O)O)C[C@H](O)[C@@H](NC(=O)CO)[C@H]([C@H](O)[C@H](O)CNC(=O)Cc2ccc(C(F)(F)F)cc2)O1 ; C#CCOCCCCCCO[C@]1(C(=O)O)C[C@H](O)[C@@H](NC(=O)CO)[C@H]([C@H](O)[C@H](O)CN)O1 ; O=C(Cc1ccc(C(F)(F)F)cc1)ON1C(=O)CCC1=O ; C#CCOCCCCCCO[C@]1(C(=O)O)C[C@H](O)[C@@H](NC(=O)CO)[C@H]([C@H](O)[C@H](O)CNC(=O)Cc2ccc(C(F)(F)F)cc2)O1]

### Synthesis of (2R,4S,5R,6R)-6-((1R,2R)-1,2-dihydroxy-3-(2-(4-(trifluoromethyl)phenyl)acetamido)propyl)-4-hydroxy-5-(2-hydroxyacetamido)-2-((6-(prop-2-yn-1-yloxy)hexyl)oxy)tetrahydro-2H-pyran-2-carboxylic acid (Cpd. 158)

To a stirred solution of (2R,4S,5R,6R)-6-((1R,2R)-3-amino-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (1, 0.100 g, 0.216 mmol) in N,N-dimethylformamide (2.0 mL) were added N,N-diisopropylethylamine (0.188 mL, 1.08 mmol) and 2,5-dioxopyrrolidin-1-yl 2-(4-(trifluoromethyl)phenyl)acetate (2, 0.065 g, 0.216 mmol) was added at 0° C. then stirred at room temperature for 4 h. After completion, the reaction mixture was concentrated, and the crude residue obtained was purified by prep HPLC using acetonitrile in water (+0.1% TFA) as eluents to afford the desired product (2R,4S,5R,6R)-6-((1R,2R)-1,2-dihydroxy-3-(2-(4-(trifluoromethyl)phenyl)acetamido)propyl)-4-hydroxy-5-(2-hydroxyacetamido)-2-((6-(prop-2-yn-1-yloxy)hexyl)oxy)tetrahydro-2H-pyran-2-carboxylic acid (Cpd. 158). Yield: 0.068 g, 49%; LCMS (ESI) m/z 649.45 [M+H]+. ¹H NMR (400 MHz, MeOD-d4): δ 7.60 (d, J=8.4 Hz, 2H), 7.49 (d, J=8.0 Hz, 2H), 4.12 (d, J=2.4 Hz, 2H), 4.01 (s, 2H), 3.91-3.67 (m, 6H), 3.63 (s, 2H), 3.51 (t, J=6.4 Hz, 2H), 3.42-3.37 (m, 2H), 3.26-3.22 (m, 1H), 2.81 (t, J=2.0 Hz, 1H), 2.71 (dd, J=12.4 Hz & 3.6 Hz, 1H), 1.73 (t, J=12.4 Hz, 1H), 1.57-1.55 (m, 4H), 1.38-1.36 (m, 4H).

### (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Compound 157)

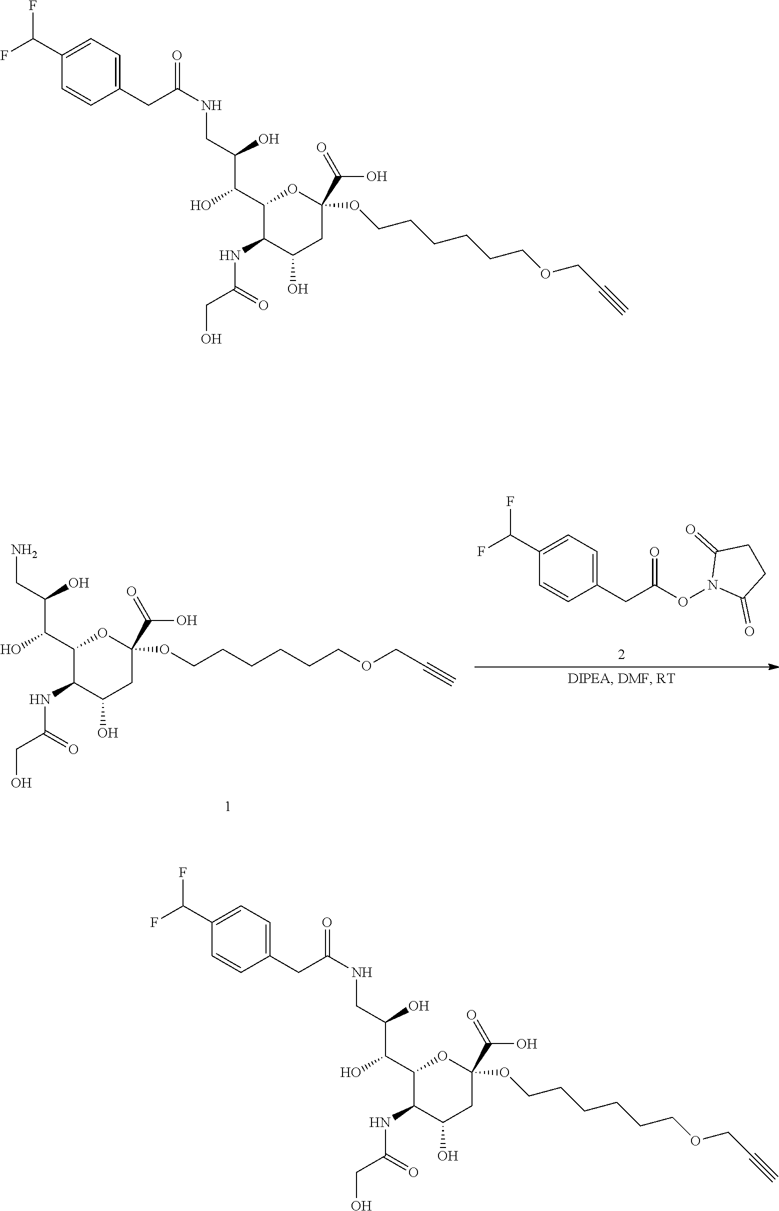 [SMILES: C#CCOCCCCCCO[C@]1(C(=O)O)C[C@H](O)[C@@H](NC(=O)CO)[C@H]([C@H](O)[C@H](O)CNC(=O)Cc2ccc(C(F)F)cc2)O1 ; C#CCOCCCCCCO[C@]1(C(=O)O)C[C@H](O)[C@@H](NC(=O)CO)[C@H]([C@H](O)[C@H](O)CN)O1 ; O=C(Cc1ccc(C(F)F)cc1)ON1C(=O)CCC1=O ; C#CCOCCCCCCO[C@]1(C(=O)O)C[C@H](O)[C@@H](NC(=O)CO)[C@H]([C@H](O)[C@H](O)CNC(=O)Cc2ccc(C(F)F)cc2)O1]

### Synthesis of (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Cpd. 157)

To a stirred solution of (2R,4S,5R,6R)-6-((1R,2R)-3-amino-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (1, 0.200 g, 0.432 mmol) in N,N-dimethylformamide (2.0 mL) were added N,N-diisopropylethylamine (0.376 mL, 2.16 mmol) and 2,5-dioxopyrrolidin-1-yl 2-(4-(difluoromethyl)phenyl)acetate (2, 0.122 g, 0.432 mmol) at 0° C. then stirred at room temperature for 4 h. After completion, the reaction mixture was concentrated, and the crude residue obtained was purified by prep HPLC using acetonitrile in water (+0.1% TFA) as eluents to afford the desired product (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Cpd. 157). Yield: 0.129 g, 47%; LCMS (ESI) m/z 631.40 [M+H]+. ¹H NMR (400 MHz, MeOD-d4): δ 7.48 (d, J=8.0 Hz, 2H), 7.42 (d, J=8.0 Hz, 2H), 6.73 (t, J=56.4 Hz, 1H), 4.12 (d, J=2.0 Hz, 2H), 4.01 (s, 2H), 3.91-3.86 (m, 1H), 3.85-3.73 (m, 3H), 3.71-3.63 (m, 2H), 3.60 (s, 2H), 3.51 (t, J=6.4 Hz, 2H), 3.43-3.36 (m, 2H), 3.25-3.22 (m, 1H), 2.80 (t, J=2.0 Hz, 1H), 2.72 (dd, J=12.4 & 3.6 Hz, 1H), 1.71 (t, J=12.0 Hz, 1H), 1.57-1.55 (m, 4H), 1.40-1.36 (m, 4H).

### (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((6-((1-(15-oxo-15-(perfluorophenoxy)-3,6,9,12-tetraoxapentadecyl)-1H-1,2,3-triazol-4-yl)methoxy)hexyl)oxy)tetrahydro-2H-pyran-2-carboxylic acid (Compound 157P)

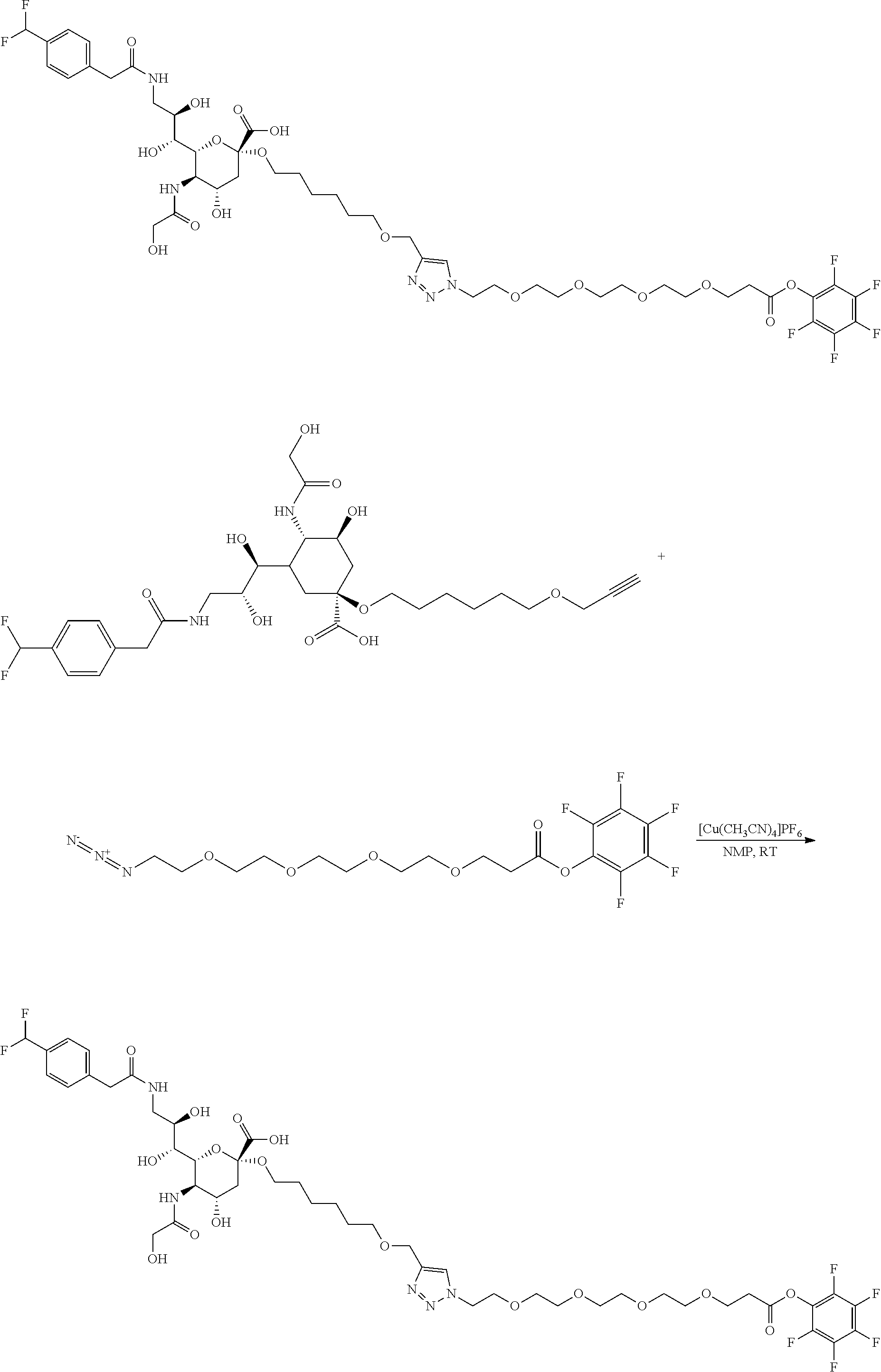

### Synthesis of (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((6-((1-(15-oxo-15-(perfluorophenoxy)-3,6,9,12-tetraoxapentadecyl)-1H-1,2,3-triazol-4-yl)methoxy)hexyl)oxy)tetrahydro-2H-pyran-2-carboxylic acid (157P)

To a solution of Azido-PEG4-PFP-ester (53.7 mg, 117 umol) in anhydrous NMP (690 uL) was added (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (157, 66.2 mg, 105 umol) in anhydrous NMP (690 uL), followed by Tetrakis(acetonitrile)copper(l) hexafluorophosphate (58.6 mg, 157 umol). The vial was sealed and the mixture was stirred for 1 hour at room temperature. The mixture was filtered through a 0.2 μm PTFE filter, a few drops of acetic acid were added, and the filtrate was purified by C18 reverse phase column chromatography using acetonitrile and water (+0.1% TFA) as eluents. The fractions containing the desired product were lyophilized to afford 157P (61.3 mg, 54%) as a white solid. Yield: 61.3 mg, 54%; LCMS (ESI) m/z 1088 [M+H]⁺. ¹H NMR (500 MHz, DMSO-d₆): 8.02 (s, 1H), 8.02 (t, J=5.5 Hz, 1H), 7.85 (d, J=8.0 Hz, 2H), 7.47 (d, J=7.5 Hz, 2H), 7.38 (d, J=7.5 Hz, 2H), 6.98 (t, J=56.0 Hz, 1H), 5.53 (s, br, 1H), 4.95 (s, br, 1H), 4.70 (s, br, 1H), 4.50 (t, J=5.5 Hz, 2H), 4.46 (s, 2H), 4.29 (s, br, 1H), 3.87 (m, 2H), 3.82-3.78 (m, 1H), 3.80 (t, J=5.5 Hz, 2H), 3.68-3.65 (m, 1H), 3.64-3.58 (m, 1H), 3.56-3.45 (m, 14H), 3.40 (t, J=6.5 Hz, 2H), 3.29-3.26 (m, 2H), 3.22-3.20 (m, 1H), 3.01 (t, J=5.5 Hz, 2H), 2.98-2.92 (m, 1H), 1.54-1.43 (m, 5H), 1.26-1.24 (m, 4H).

### (2R,4S,5R,6R)-6-((1R,2R)-3-(2-(4-(difluoromethyl)phenyl)acetamido)-1,2-dihydroxypropyl)-2-((6-((1-(18-(2,5-dioxo-2,5-dihydro-1H-pyrrol-1-yl)-15-oxo-3,6,9,12-tetraoxa-16-azaoctadecyl)-1H-1,2,3-triazol-4-yl)methoxy)hexyl)oxy)-4-hydroxy-5-(2-hydroxyacetamido)tetrahydro-2H-pyran-2-carboxylic acid (Compound 157M)

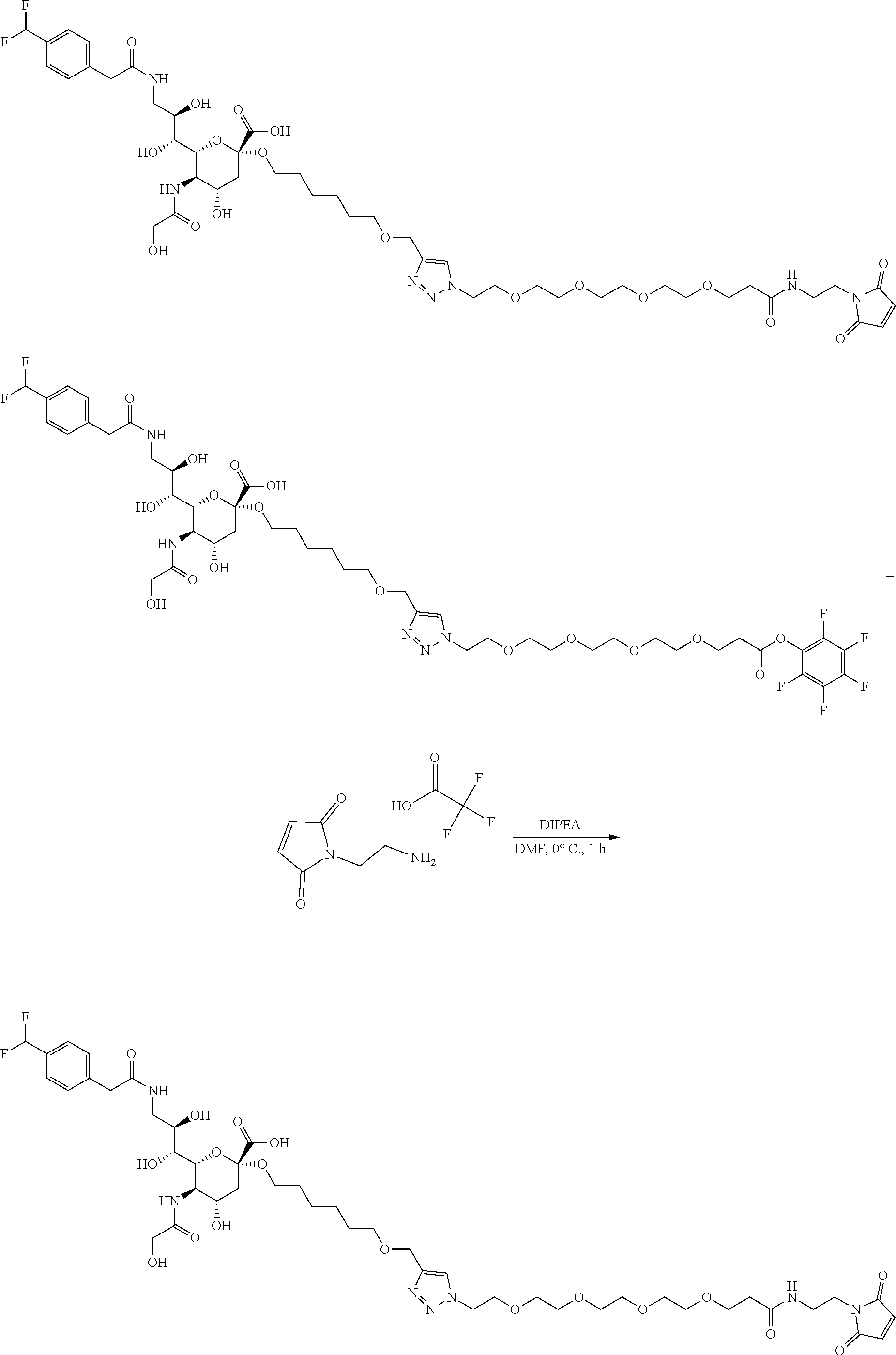 [SMILES: O=C(CCOCCOCCOCCOCCn1cc(COCCCCCCO[C@]2(C(=O)O)C[C@H](O)[C@@H](NC(=O)CO)[C@H]([C@H](O)[C@H](O)CNC(=O)Cc3ccc(C(F)F)cc3)O2)nn1)NCCN1C(=O)C=CC1=O ; O=C(Cc1ccc(C(F)F)cc1)NC[C@@H](O)[C@@H](O)[C@@H]1O[C@@](OCCCCCCOCc2cn(CCOCCOCCOCCOCCC(=O)Oc3c(F)c(F)c(F)c(F)c3F)nn2)(C(=O)O)C[C@H](O)[C@H]1NC(=O)CO ; NCCN1C(=O)C=CC1=O ; O=C(O)C(F)(F)F ; O=C(CCOCCOCCOCCOCCn1cc(COCCCCCCO[C@]2(C(=O)O)C[C@H](O)[C@@H](NC(=O)CO)[C@H]([C@H](O)[C@H](O)CNC(=O)Cc3ccc(C(F)F)cc3)O2)nn1)NCCN1C(=O)C=CC1=O]

### Synthesis of (2R,4S,5R,6R)-6-((1R,2R)-3-(2-(4-(difluoromethyl)phenyl)acetamido)-1,2-dihydroxypropyl)-2-((6-((1-(18-(2,5-dioxo-2,5-dihydro-1H-pyrrol-1-yl)-15-oxo-3,6,9,12-tetraoxa-16-azaoctadecyl)-1H-1,2,3-triazol-4-yl)methoxy)hexyl)oxy)-4-hydroxy-5-(2-hydroxyacetamido)tetrahydro-2H-pyran-2-carboxylic acid (157M)

To a solution of (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((6-((1-(15-oxo-15-(perfluorophenoxy)-3,6,9,12-tetraoxapentadecyl)-1H-1,2,3-triazol-4-yl)methoxy)hexyl)oxy)tetrahydro-2H-pyran-2-carboxylic acid (157P, 14.4 mg, 13.2 umol) in DMF (319 uL) was added n-(2-aminoethyl)maleimide trifluoroacetate salt (3.87 mg, 15.2 umol). The mixture was cooled down to 0° C. and stirred for 10 min. DIEA (6.92 uL, 39.7 umol) was added and the mixture was stirred for 1 h at 0° C. The reaction mixture was filtered and purified by preparatory HPLC using acetonitrile and water (+0.05% TFA) as eluents. The fractions containing the desired product were combined and lyophilized to dryness to afford 157M as a white solid. Yield: 11.5 mg, 83%; LCMS (ESI) m/z 1044 [M+H]⁺. ¹H NMR (500 MHz, DMSO-d₆): 8.03 (s, 1H), 8.02 (t, J=5.5 Hz, 1H), 7.93 (t, J=6.0 Hz, 1H), 7.85 (d, J=8.0 Hz, 2H), 7.47 (d, J=7.5 Hz, 2H), 7.38 (d, J=7.5 Hz, 2H), 6.99 (s, 2H), 6.98 (t, J=56.0 Hz, 1H), 4.50 (t, J=5.5 Hz, 2H), 4.46 (s, 2H), 3.87 (m, 2H), 3.83-3.79 (m, 1H), 3.80 (t, J=5.5 Hz, 2H), 3.69-3.65 (m, 1H), 3.64-3.60 (m, 1H), 3.56-3.43 (m, 16H), 3.29-3.26 (m, 3H), 3.22-3.16 (m, 4H), 2.98-2.93 (m, 1H), 2.21 (t, J=6.5 Hz, 2H), 1.54-1.43 (m, 5H), 1.26-1.24 (m, 4H).

### (2R,4S,5R,6R)-6-((1R,2R)-1,2-dihydroxy-3-(2-(4-(trifluoromethoxy)phenyl)acetamido)propyl)-4-hydroxy-5-(2-hydroxyacetamido)-2-((6-(prop-2-yn-1-yloxy)hexyl)oxy)tetrahydro-2H-pyran-2-carboxylic acid (Compound 156)

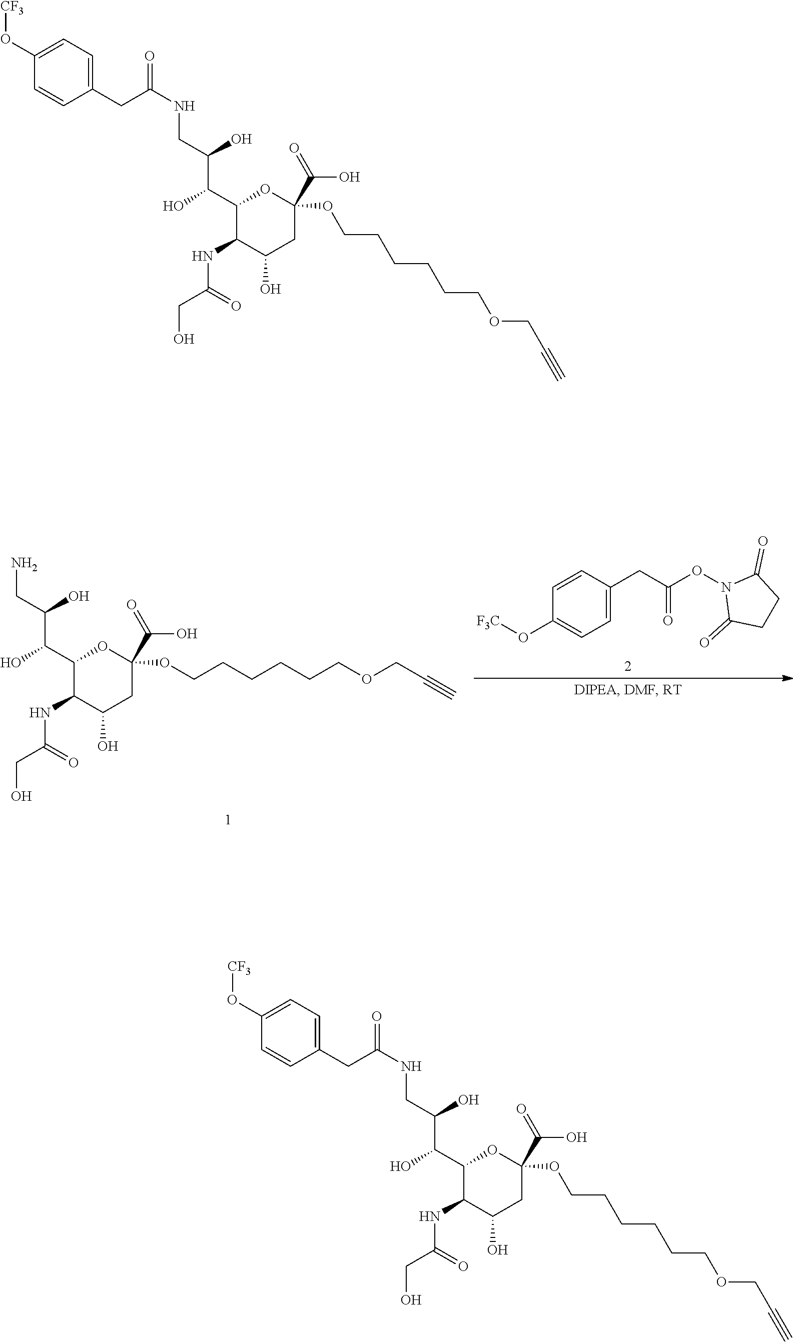 [SMILES: C#CCOCCCCCCO[C@]1(C(=O)O)C[C@H](O)[C@@H](NC(=O)CO)[C@H]([C@H](O)[C@H](O)CNC(=O)Cc2ccc(OC(F)(F)F)cc2)O1 ; C#CCOCCCCCCO[C@]1(C(=O)O)C[C@H](O)[C@@H](NC(=O)CO)[C@H]([C@H](O)[C@H](O)CN)O1 ; O=C(Cc1ccc(OC(F)(F)F)cc1)ON1C(=O)CCC1=O ; C#CCOCCCCCCO[C@]1(C(=O)O)C[C@H](O)[C@@H](NC(=O)CO)[C@H]([C@H](O)[C@H](O)CNC(=O)Cc2ccc(OC(F)(F)F)cc2)O1]

### Synthesis of (2R,4S,5R,6R)-6-((1R,2R)-1,2-dihydroxy-3-(2-(4-(trifluoromethoxy)phenyl)acetamido)propyl)-4-hydroxy-5-(2-hydroxyacetamido)-2-((6-(prop-2-yn-1-yloxy)hexyl)oxy)tetrahydro-2H-pyran-2-carboxylic acid (Cpd. 156)

To a stirred solution of (2R,4S,5R,6R)-6-((1R,2R)-3-amino-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (1, 0.100 g, 0.216 mmol) in N,N-dimethylformamide (2.0 mL) were added N,N-diisopropylethylamine (0.188 mL, 1.08 mmol) and 2,5-dioxopyrrolidin-1-yl 2-(4-(trifluoromethoxy)phenyl)acetate (2, 0.068 g, 0.216 mmol) was added at 0° C. then stirred at room temperature for 4 h. After completion, the reaction mixture was concentrated, and the crude residue obtained was purified by prep HPLC using acetonitrile in water (+0.1% TFA) as eluents to afford the desired product (2R,4S,5R,6R)-6-((1R,2R)-1,2-dihydroxy-3-(2-(4-(trifluoromethoxy)phenyl)acetamido)propyl)-4-hydroxy-5-(2-hydroxyacetamido)-2-((6-(prop-2-yn-1-yloxy)hexyl)oxy)tetrahydro-2H-pyran-2-carboxylic acid (Cpd. 156). Yield: 0.037 g, 26%; LCMS (ESI) m/z 665.30 [M+H]+. ¹H NMR (400 MHz, MeOD-d4): δ 7.39 (d, J=8.8 Hz, 2H), 7.21 (d, J=8.4 Hz, 2H), 4.12 (d, J=2.4 Hz, 2H), 4.01 (s, 2H), 3.90-3.63 (m, 6H), 3.57 (s, 2H), 3.51 (t, J=6.4 Hz, 2H), 3.41-3.37 (m, 2H), 3.27-3.21 (m, 1H), 2.81 (t, J=2.4 Hz, 1H), 2.72 (dd, J=12.0 Hz & 3.2 Hz, 1H), 1.72 (t, J=12.0 Hz, 1H), 1.57-1.53 (m, 4H), 1.38-1.36 (m, 4H).

### (2R,4S,5R,6R)-6-((1R,2R)-3-(2-(4-chloro-3-(trifluoromethyl)phenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Compound 127)

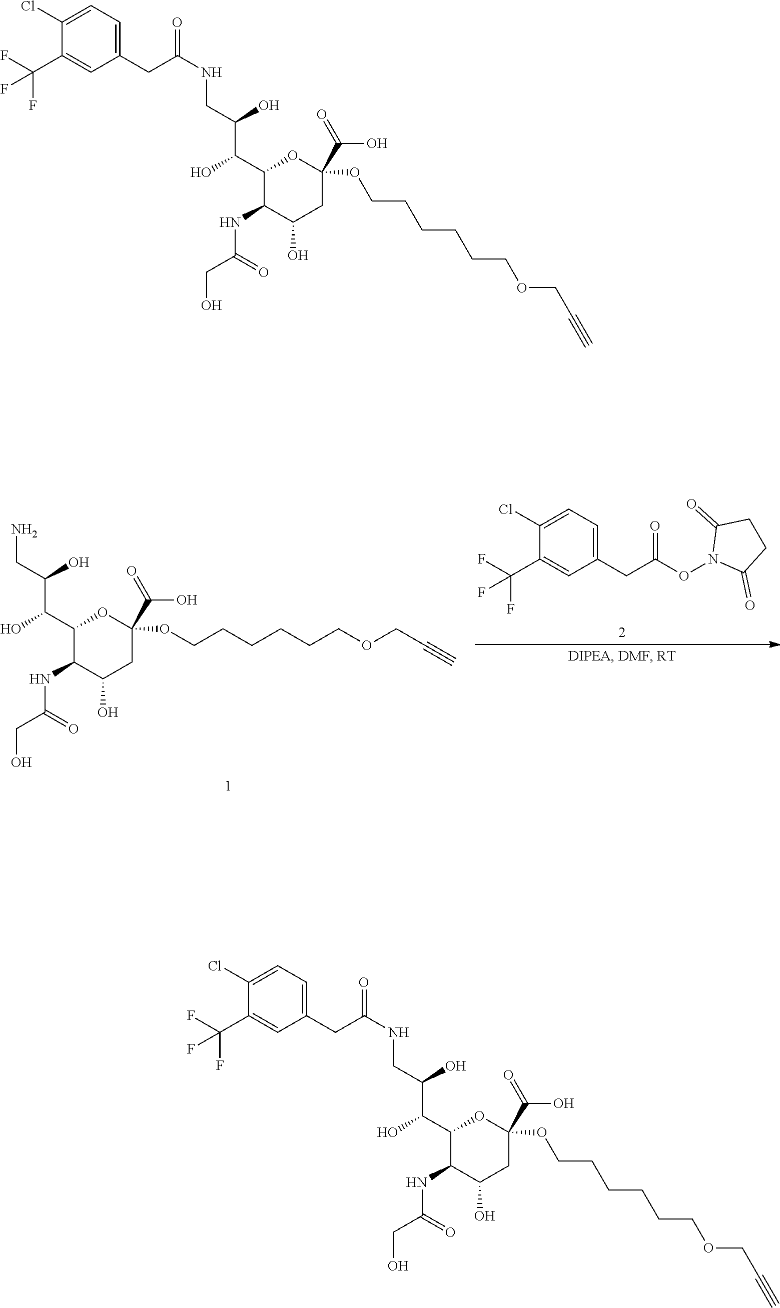 [SMILES: C#CCOCCCCCCO[C@]1(C(=O)O)C[C@H](O)[C@@H](NC(=O)CO)[C@H]([C@H](O)[C@H](O)CNC(=O)Cc2ccc(Cl)c(C(F)(F)F)c2)O1 ; C#CCOCCCCCCO[C@]1(C(=O)O)C[C@H](O)[C@@H](NC(=O)CO)[C@H]([C@H](O)[C@H](O)CN)O1 ; O=C(Cc1ccc(Cl)c(C(F)(F)F)c1)ON1C(=O)CCC1=O ; C#CCOCCCCCCO[C@]1(C(=O)O)C[C@H](O)[C@@H](NC(=O)CO)[C@H]([C@H](O)[C@H](O)CNC(=O)Cc2ccc(Cl)c(C(F)(F)F)c2)O1]

### Synthesis of (2R,4S,5R,6R)-6-((1R,2R)-3-(2-(4-chloro-3-(trifluoromethyl)phenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Cpd. 127)

To a stirred solution of (2R,4S,5R,6R)-6-((1R,2R)-3-amino-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (1, 0.100 g, 0.216 mmol) in N,N-dimethylformamide (2.0 mL) were added N,N-diisopropylethylamine (0.188 mL, 1.08 mmol) and 2,5-dioxopyrrolidin-1-yl 2-(4-chloro-3-(trifluoromethyl)phenyl)acetate (2, 0.072 g, 0.216 mmol) was added at 0° C. then stirred at room temperature for 4 h. After completion, the reaction mixture was concentrated, and the crude residue obtained was purified by prep HPLC using acetonitrile in water (+0.1% TFA) as eluents to afford the desired product (2R,4S,5R,6R)-6-((1R,2R)-3-(2-(4-chloro-3-(trifluoromethyl)phenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Cpd. 127). Yield: 0.055 g, 37%; LCMS (ESI) m/z 683.35 [M+H]+. ¹H NMR (400 MHz, MeOD-d4): δ 7.74 (s, 1H), 7.56-7.51 (m, 2H), 4.12 (d, J=2.4 Hz, 2H), 4.01 (s, 2H), 3.88-3.64 (m, 6H), 3.61 (s, 2H), 3.51 (t, J=6.4 Hz, 2H), 3.40-3.36 (m, 2H), 3.25-3.20 (m, 1H), 2.81 (t, J=2.4 Hz, 1H), 2.72 (dd, J=12.4 Hz & 4.0 Hz, 1H), 1.74 (t, J=6.4 Hz, 1H), 1.57-1.52 (m, 4H), 1.39-1.35 (m, 4H).

### (2R,4S,5R,6R)-6-((1R,2R)-1,2-dihydroxy-3-(2-(3-(trifluoromethoxy)phenyl)acetamido)propyl)-4-hydroxy-5-(2-hydroxyacetamido)-2-((6-(prop-2-yn-1-yloxy)hexyl)oxy)tetrahydro-2H-pyran-2-carboxylic acid (Compound 155)

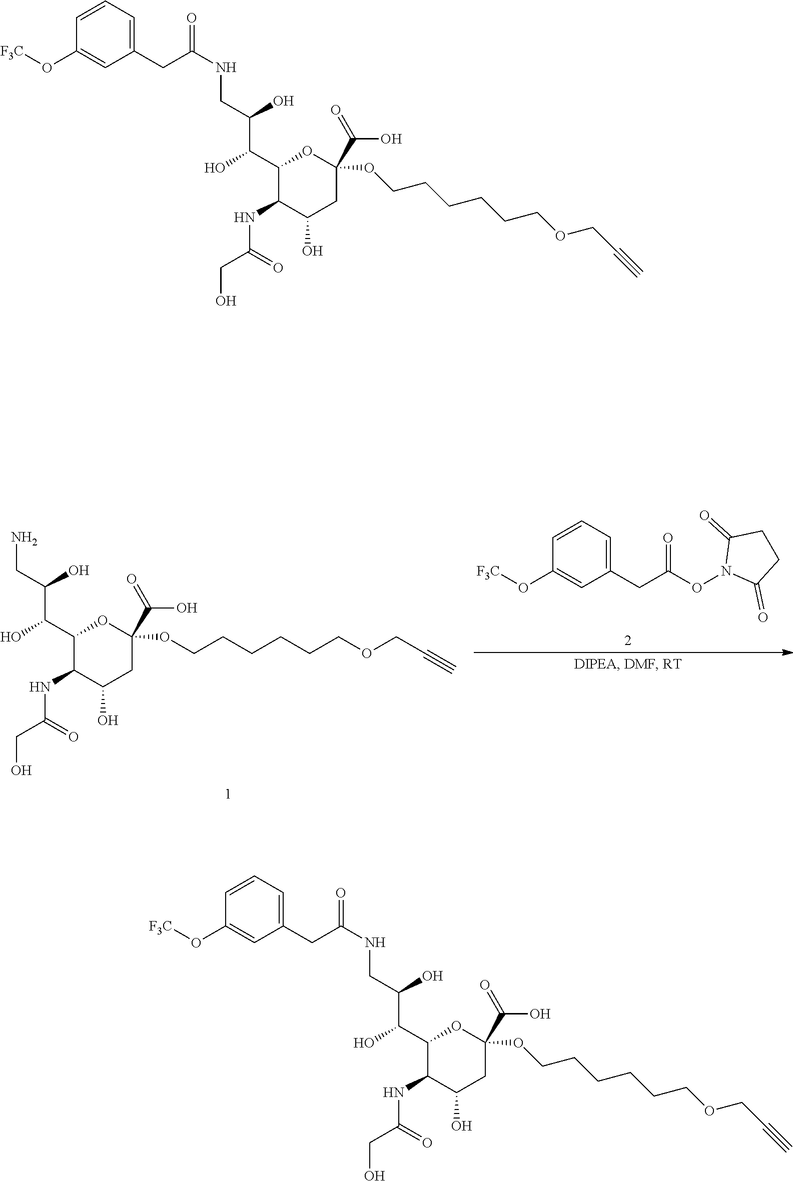 [SMILES: C#CCOCCCCCCO[C@]1(C(=O)O)C[C@H](O)[C@@H](NC(=O)CO)[C@H]([C@H](O)[C@H](O)CNC(=O)Cc2cccc(OC(F)(F)F)c2)O1 ; C#CCOCCCCCCO[C@]1(C(=O)O)C[C@H](O)[C@@H](NC(=O)CO)[C@H]([C@H](O)[C@H](O)CN)O1 ; O=C(Cc1cccc(OC(F)(F)F)c1)ON1C(=O)CCC1=O ; C#CCOCCCCCCO[C@]1(C(=O)O)C[C@H](O)[C@@H](NC(=O)CO)[C@H]([C@H](O)[C@H](O)CNC(=O)Cc2cccc(OC(F)(F)F)c2)O1]

### Synthesis of (2R,4S,5R,6R)-6-((1R,2R)-1,2-dihydroxy-3-(2-(3-(trifluoromethoxy)phenyl)acetamido)propyl)-4-hydroxy-5-(2-hydroxyacetamido)-2-((6-(prop-2-yn-1-yloxy)hexyl)oxy)tetrahydro-2H-pyran-2-carboxylic acid (Cpd. 155)

To a stirred solution of (2R,4S,5R,6R)-6-((1R,2R)-3-amino-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (1, 0.100 g, 0.216 mmol) in N,N-dimethylformamide (2.0 mL) were added N,N-diisopropylethylamine (0.188 mL, 1.08 mmol) and 2,5-dioxopyrrolidin-1-yl 2-(3-(trifluoromethoxy)phenyl)acetate (2, 0.068 g, 0.216 mmol) was added at 0° C. then stirred at room temperature for 4 h. After completion, the reaction mixture was concentrated, and the crude residue obtained was purified by prep HPLC using acetonitrile in water (+0.1% TFA) as eluents to afford the desired product (2R,4S,5R,6R)-6-((1R,2R)-1,2-dihydroxy-3-(2-(3-(trifluoromethoxy)phenyl)acetamido)propyl)-4-hydroxy-5-(2-hydroxyacetamido)-2-((6-(prop-2-yn-1-yloxy)hexyl)oxy)tetrahydro-2H-pyran-2-carboxylic acid (Cpd. 155). Yield: 0.046 g, 32%; LCMS (ESI) m/z 665.40 [M+H]+. ¹H NMR (400 MHz, MeOD-d4): δ 7.40 (t, J=7.6 Hz, 1H), 7.30 (d, J=8.0 Hz, 1H), 7.25 (s, 1H), 7.15 (d, J=8.4 Hz, 1H), 4.12 (d, J=2.4 Hz, 2H), 4.01 (s, 2H), 3.90-3.85 (m, 1H), 3.83-3.73 (m, 3H), 3.71-3.63 (m, 2H), 3.59 (s, 2H), 3.51 (t, J=6.4 Hz, 2H), 3.42-3.36 (m, 2H), 3.25-3.20 (m, 1H), 2.81 (t, J=2.4 Hz, 1H), 2.71 (dd, J=12.4 & 3.6 Hz, 1H), 1.73 (t, J=12.0 Hz, 1H), 1.57-1.53 (m, 4H), 1.38-1.35 (m, 4H).

### (2R,4S,5R,6R)-6-((1R,2R)-3-(2-(4-chlorophenyl)acetamido)-1,2-dihydroxypropyl)-4-hydroxy-5-(3-(2-hydroxyethyl)ureido)-2-((6-(prop-2-yn-1-yloxy)hexyl)oxy)tetrahydro-2H-pyran-2-carboxylic acid (Compound 185)

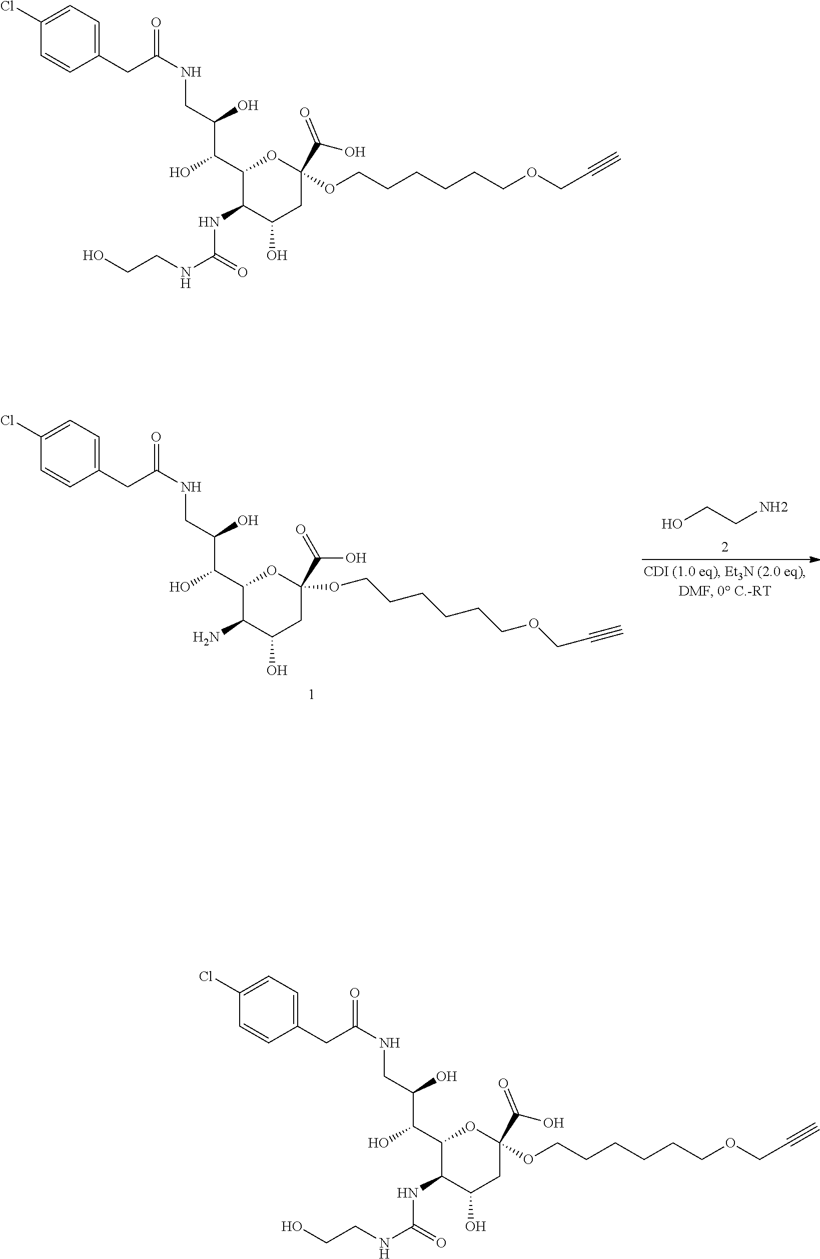

### Synthesis of (2R,4S,5R,6R)-6-((1R,2R)-3-(2-(4-chlorophenyl)acetamido)-1,2-dihydroxypropyl)-4-hydroxy-5-(3-(2-hydroxyethyl)ureido)-2-((6-(prop-2-yn-1-yloxy)hexyl)oxy)tetrahydro-2H-pyran-2-carboxylic acid (Cpd. 185)

To a stirred solution of (2R,4S,5R,6R)-5-amino-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (1, 0.080 g, 0.144 mmol) in anhydrous dimethylformamide (2 mL) was added 1,1′-Carbonyldiimidazole (0.023 g, 0.144 mmol) and trimethylamine (0.029 mL, 0.287 mmol) at 0° C. followed by addition of 2-aminoethanol (0.0175 mL, 0.287 mmol) was stirred at room temperature for 12 h. After completion, the reaction mixture was concentrated. The crude residue obtained was purified by Prep-HPLC using acetonitrile in water (+0.1% TFA) as eluents to afford the desired product (2R,4S,5R,6R)-6-((1R,2R)-3-(2-(4-chlorophenyl)acetamido)-1,2-dihydroxypropyl)-4-hydroxy-5-(3-(2-hydroxyethyl)ureido)-2-((6-(prop-2-yn-1-yloxy)hexyl)oxy)tetrahydro-2H-pyran-2-carboxylic acid (Cpd. 185). Yield: 0.045 g, 48.65%; LCMS m/z 644.35 [M+H]+; ¹H-NMR (400 MHz, Methanol-d₄): δ 7.29 (bs, 4H), 4.12 (d, J=2.4 Hz, 2H), 3.90-3.85 (m, 1H), 3.81-3.71 (m, 1H), 3.67-3.46 (m, 11H), 3.41-3.37 (m, 1H), 3.29-3.21 (m, 3H), 2.81 (t, J=2.4 Hz, 1H), 2.72-2.68 (dd, J=4.4 Hz, 12.8 Hz, 1H), 1.71 (t, J=12.4 Hz, 1H), 1.58-1.52 (m, 4H), 1.38-1.35 (m, 4H).

### (2R,4S,5R,6R)-6-((1R,2R)-3-(2-(4-chlorophenyl)acetamido)-1,2-dihydroxypropyl)-5-(3-(cyclopropylmethyl)ureido)-4-hydroxy-2-((6-(prop-2-yn-1-yloxy)hexyl)oxy)tetrahydro-2H-pyran-2-carboxylic acid (Compound 94)

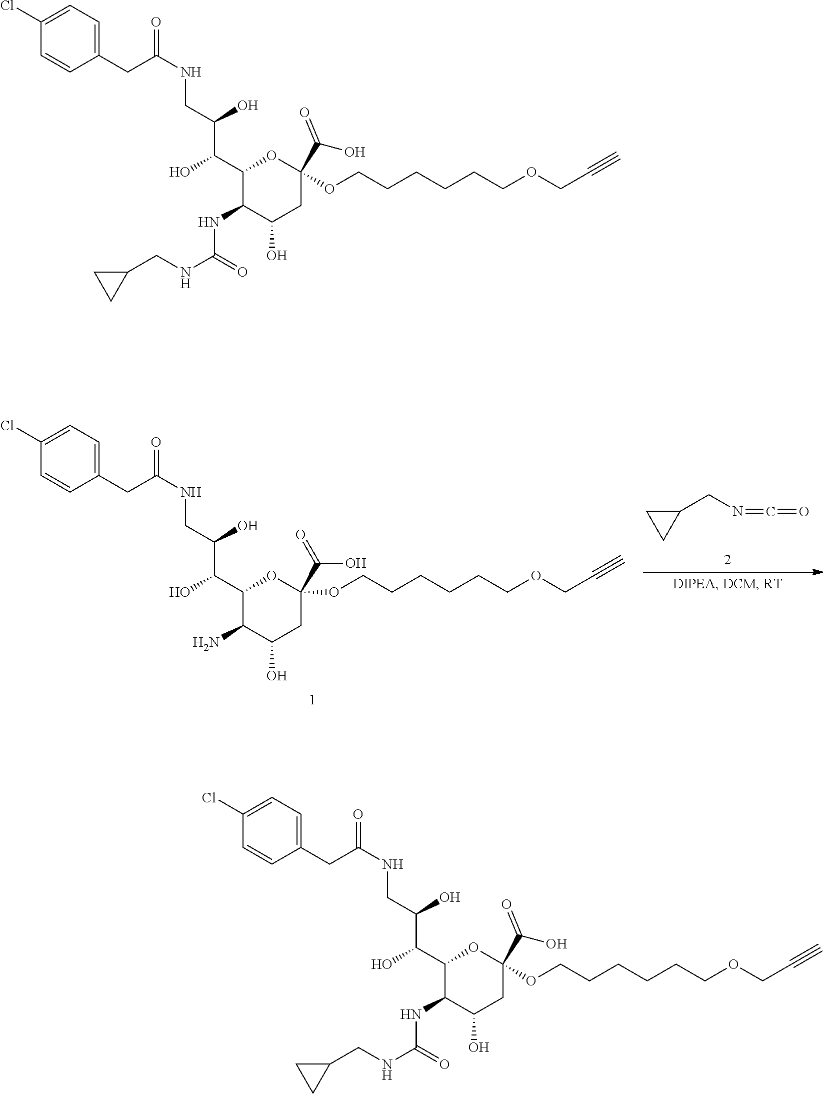

### Synthesis of (2R,4S,5R,6R)-6-((1R,2R)-3-(2-(4-chlorophenyl)acetamido)-1,2-dihydroxypropyl)-5-(3-(cyclopropylmethyl)ureido)-4-hydroxy-2-((6-(prop-2-yn-1-yloxy)hexyl)oxy)tetrahydro-2H-pyran-2-carboxylic acid (Cpd. 94)

To a stirred solution of (2R,4S,5R,6R)-5-amino-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (1, 0.06 g, 0.11 mmol) in dry dichloromethane (1.0 mL) were sequentially added (isocyanatomethyl)cyclopropane (0.015 g, 0.11 mmol) and N,N-Diisopropylethylamine (0.09 mL, 0.55 mmol) dropwise was stirred for 12 h at room temperature. After completion, the reaction mixture was concentrated. The crude residue obtained was purified by Prep-HPLC using acetonitrile in water (+0.1% TFA) as eluents to afford the desired product (2R,4S,5R,6R)-6-((1R,2R)-3-(2-(4-chlorophenyl)acetamido)-1,2-dihydroxypropyl)-5-(3-(cyclopropylmethyl)ureido)-4-hydroxy-2-((6-(prop-2-yn-1-yloxy)hexyl)oxy)tetrahydro-2H-pyran-2-carboxylic acid (Cpd. 94). Yield: 0.035 g, 49%; LCMS m/z 654.50 [M+H]+; ¹H-NMR (400 MHz, Methanol-d₄): δ 7.31-7.27 (m, 4H), 4.12 (d, J=2.4 Hz, 2H), 3.89 (td, J=8.0 Hz & 2.8 Hz, 1H), 3.75-3.72 (m, 1H), 3.65 (dd, J=13.6 Hz & 2.8 Hz, 1H), 3.59-3.46 (m, 7H), 3.41-3.36 (m, 2H), 3.28-3.24 (m, 1H), 2.98 (d, J=7.2 Hz, 2H), 2.82 (t, J=2.4 Hz, 1H), 2.70 (dd, J=12.8 Hz & 4.8 Hz, 1H), 1.72 (t, J=12.0 Hz, 1H), 1.57-1.54 (m, 4H), 1.38-1.36 (m, 4H), 0.96-0.91 (m, 1H), 0.49-0.44 (m, 2H), 0.20-0.16 (m, 2H).

### (2R,4S,5R,6R)-6-((1R,2R)-3-(2-(4-chlorophenyl)acetamido)-1,2-dihydroxypropyl)-5-ethanethioamido-4-hydroxy-2-((6-(prop-2-yn-1-yloxy)hexyl)oxy)tetrahydro-2H-pyran-2-carboxylic acid (Compound 26)

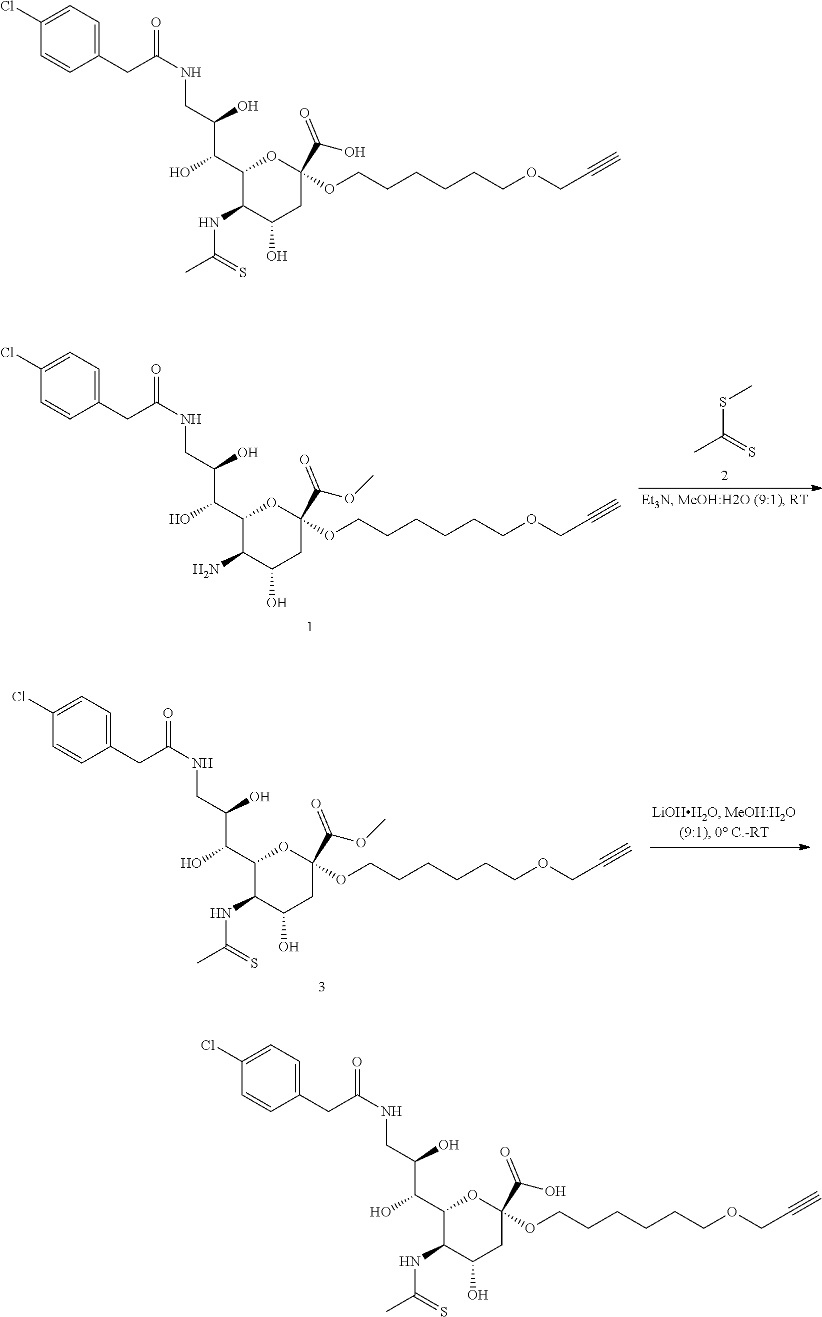

### Synthesis of methyl (2R,4S,5R,6R)-6-((1R,2R)-3-(2-(4-chlorophenyl)acetamido)-1,2-dihydroxypropyl)-5-ethanethioamido-4-hydroxy-2-((6-(prop-2-yn-1-yloxy)hexyl)oxy)tetrahydro-2H-pyran-2-carboxylate (3)

To a stirred solution of methyl (2R,4S,5R,6R)-5-amino-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylate (1, 0.15 g, 0.27 mmol) in a mixture of methanol and water (9:1; 10.0 mL) were sequentially added methyl ethanedithioate (0.28 g, 2.69 mmol) and triethylamine (0.38 mL, 2.69 mmol) dropwise at 0° C. was warmed up and stirred for 12 h at room temperature. After completion, the reaction mixture was concentrated. and triturated using diethyl ether to obtain crude residue (2R,4S,5R,6R)-6-((1R,2R)-3-(2-(4-chlorophenyl)acetamido)-1,2-dihydroxypropyl)-5-ethanethioamido-4-hydroxy-2-((6-(prop-2-yn-1-yloxy)hexyl)oxy)tetrahydro-2H-pyran-2-carboxylate (3) which was used for next step without further purification; Yield: 0.11 g, 66%; LCMS m/z 628.90 [M+H]+.

### Synthesis of (2R,4S,5R,6R)-6-((1R,2R)-3-(2-(4-chlorophenyl)acetamido)-1,2-dihydroxypropyl)-5-ethanethioamido-4-hydroxy-2-((6-(prop-2-yn-1-yloxy)hexyl)oxy)tetrahydro-2H-pyran-2-carboxylic acid (Cpd. 94)

To a stirred solution of (2R,4S,5R,6R)-6-((1R,2R)-3-(2-(4-chlorophenyl)acetamido)-1,2-dihydroxypropyl)-5-ethanethioamido-4-hydroxy-2-((6-(prop-2-yn-1-yloxy)hexyl)oxy)tetrahydro-2H-pyran-2-carboxylate (3, 0.11 g, 0.17 mmol) in methanol (5.0 mL), was added a solution of lithium hydroxide monohydrate (0.025 g, 1.05 mmol) in water (1.0 mL). The reaction mixture was stirred at room temperature for 5 h. After completion, the reaction mixture was treated with acidic resin (Dowex 50, H⁺) to pH ˜6 and the suspension was filtered. The filtrate was concentrated, and the crude residue obtained was purified by Prep-HPLC using acetonitrile in water (+0.1% TFA) as eluents to afford the desired product (2R,4S,5R,6R)-6-((1R,2R)-3-(2-(4-chlorophenyl)acetamido)-1,2-dihydroxypropyl)-5-ethanethioamido-4-hydroxy-2-((6-(prop-2-yn-1-yloxy)hexyl)oxy)tetrahydro-2H-pyran-2-carboxylic acid (Cpd. 26). Yield: 0.019 g, 18%; LCMS m/z 615.30 [M+H]+; ¹H-NMR (400 MHz, Methanol-d₄): δ 7.32-7.17 (m, 4H), 4.47 (t, J=10.0 Hz, 1H), 4.12 (d, J=2.4 Hz, 1H), 3.91-3.81 (m, 2H), 3.80-3.71 (m, 2H), 3.60 (dd, J=14.0 Hz & 3.2 Hz, 1H), 3.55-3.51 (m, 4H), 3.44-3.40 (m, 1H), 3.38-3.32 (m, 2H), 2.81 (t, J=2.4 Hz, 1H), 2.70 (dd, J=12.8 Hz & 4.8 Hz, 1H), 2.50 (s, 3H), 1.76 (t, J=12.0 Hz, 1H), 1.57-1.55 (m, 4H), 1.39-1.36 (m, 4H).

### (2R,4S,5R,6R)-6-((1R,2R)-3-(2-(4-chlorophenyl)acetamido)-1,2-dihydroxypropyl)-4-hydroxy-5-(2-phenylacetamido)-2-((6-(prop-2-yn-1-yloxy)hexyl)oxy)tetrahydro-2H-pyran-2-carboxylic acid (Compound 93)

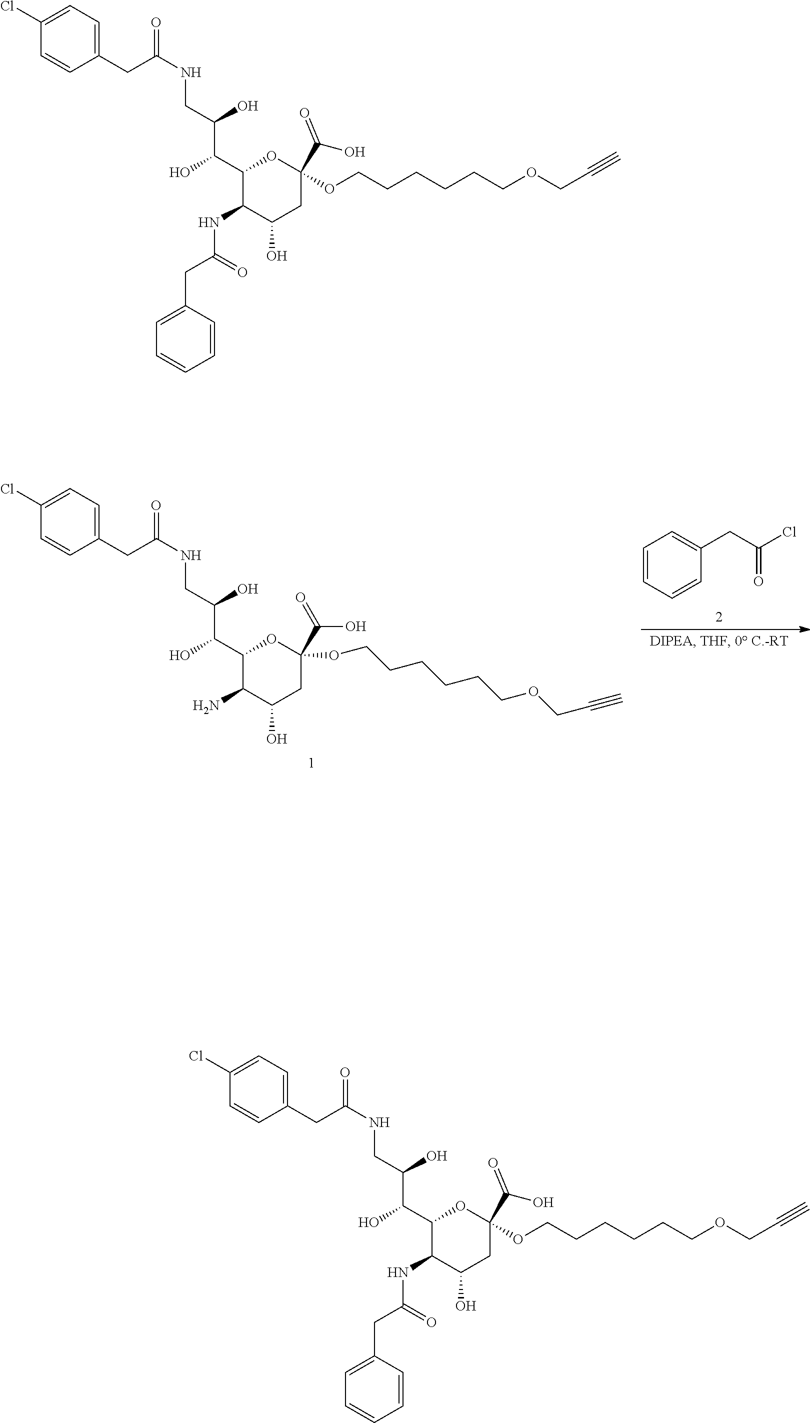 [SMILES: C#CCOCCCCCCO[C@]1(C(=O)O)C[C@H](O)[C@@H](NC(=O)Cc2ccccc2)[C@H]([C@H](O)[C@H](O)CNC(=O)Cc2ccc(Cl)cc2)O1 ; C#CCOCCCCCCO[C@]1(C(=O)O)C[C@H](O)[C@@H](N)[C@H]([C@H](O)[C@H](O)CNC(=O)Cc2ccc(Cl)cc2)O1 ; O=C(Cl)Cc1ccccc1 ; C#CCOCCCCCCO[C@]1(C(=O)O)C[C@H](O)[C@@H](NC(=O)Cc2ccccc2)[C@H]([C@H](O)[C@H](O)CNC(=O)Cc2ccc(Cl)cc2)O1]

### Synthesis of (2R,4S,5R,6R)-6-((1R,2R)-3-(2-(4-chlorophenyl)acetamido)-1,2-dihydroxypropyl)-4-hydroxy-5-(2-phenylacetamido)-2-((6-(prop-2-yn-1-yloxy)hexyl)oxy)tetrahydro-2H-pyran-2-carboxylic acid (Cpd. 93)

To a stirred solution of (2R,4S,5R,6R)-5-amino-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (1, 0.10 g, 0.180 mmol) in Tetrahydrofuran (3.0 mL) was added ethylbis(propan-2-yl)amine (0.161 mL, 0.898 mmol) and 2-phenylacetyl chloride (2, 0.027 g, 0.180 mmol) at 0° C. then stirred at room temperature for 1 h. After completion, the reaction mixture was concentrated, and the crude residue obtained was purified by prep HPLC using acetonitrile in water (+0.05% TFA) to afford the desired product (2R,4S,5R,6R)-6-((1R,2R)-3-(2-(4-chlorophenyl)acetamido)-1,2-dihydroxypropyl)-4-hydroxy-5-(2-phenylacetamido)-2-((6-(prop-2-yn-1-yloxy)hexyl)oxy)tetrahydro-2H-pyran-2-carboxylic acid (Cpd. 93). Yield: 0.030 g, 24.75%; LCMS m/z 675.40 [M+H]+; ¹H NMR (400 MHz, methanol-d4): δ 8.23 (d, J=7.6 Hz, 1H), 7.30-7.14 (m, 9H), 4.11 (d, J=2.4 Hz, 2H), 3.84-3.79 (m, 1H), 3.75-3.70 (m, 3H), 3.57-3.49 (m, 8H), 3.41-3.35 (m, 1H), 3.19-3.16 (m, 1H), 3.15-3.10 (m, 1H), 2.80 (t, J=2.0 Hz, 1H), 2.72-2.68 (dd, J=2.4 Hz, 12.0 Hz, 1H), 1.74-1.68 (m, 1H), 1.56-1.52 (m, 4H) 1.37-1.35 (m, 4H).

### (2R,4S,5R,6R)-5-benzamido-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (Compound 92)

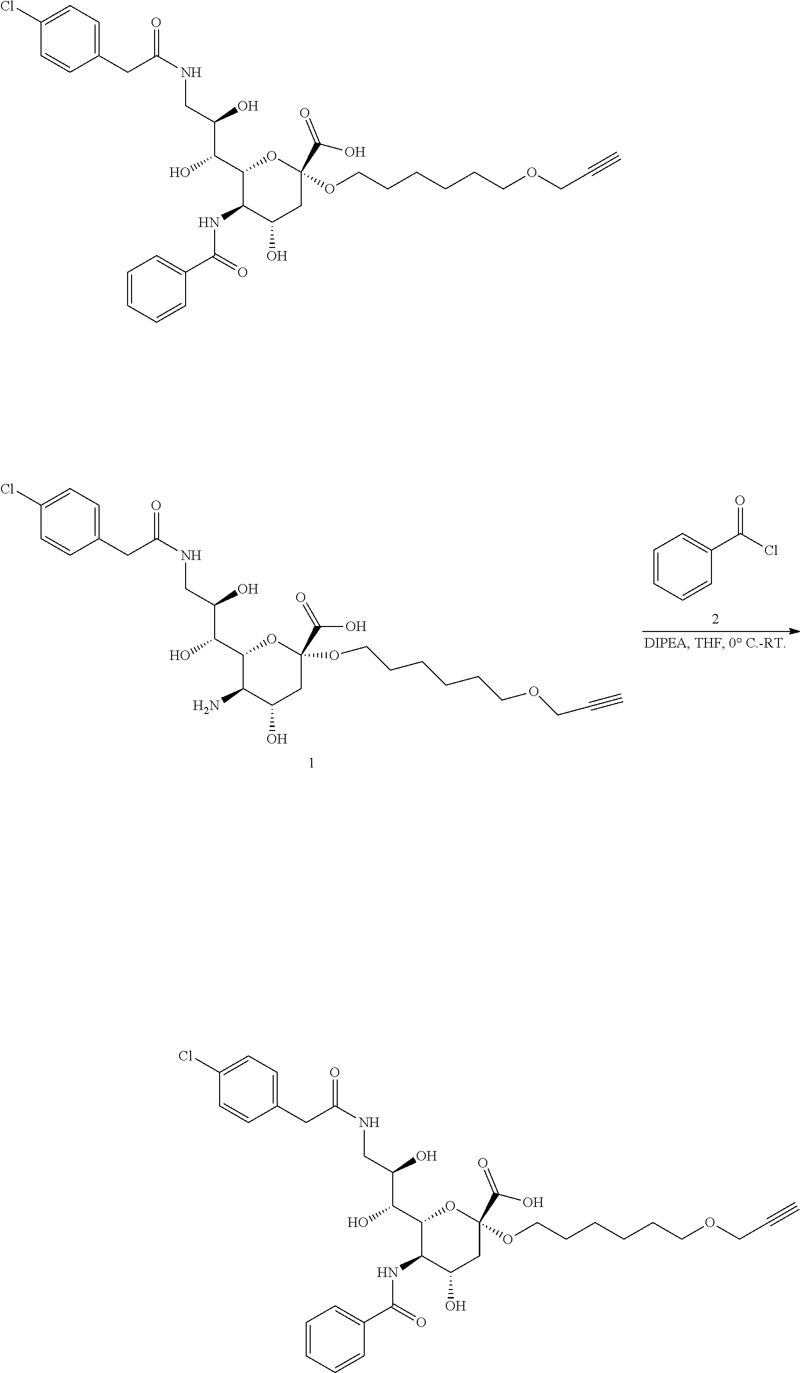 [SMILES: C#CCOCCCCCCO[C@]1(C(=O)O)C[C@H](O)[C@@H](NC(=O)c2ccccc2)[C@H]([C@H](O)[C@H](O)CNC(=O)Cc2ccc(Cl)cc2)O1 ; C#CCOCCCCCCO[C@]1(C(=O)O)C[C@H](O)[C@@H](N)[C@H]([C@H](O)[C@H](O)CNC(=O)Cc2ccc(Cl)cc2)O1 ; O=C(Cl)c1ccccc1 ; C#CCOCCCCCCO[C@]1(C(=O)O)C[C@H](O)[C@@H](NC(=O)c2ccccc2)[C@H]([C@H](O)[C@H](O)CNC(=O)Cc2ccc(Cl)cc2)O1]

### Synthesis of (2R,4S,5R,6R)-5-benzamido-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (Cpd. 92)

To a stirred solution of (2R,4S,5R,6R)-5-amino-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (1, 0.10 g, 0.180 mmol) in THF (3.0 mL) was added ethylbis(propan-2-yl)amine (0.116 mL, 0.898 mmol) and benzoyl chloride (2, 0.025 g, 0.180 mmol) at 0° C. then stirred at room temperature for 1 h. After completion, the reaction mixture was concentrated, and the crude residue obtained was purified by prep HPLC using acetonitrile in water (+0.05% TFA) to afford the desired product (2R,4S,5R,6R)-5-benzamido-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (Cpd. 92). Yield: 0.055 g, 46.35%; LCMS m/z 661.50 [M+H]+; ¹H NMR (400 MHz, methanol-d4): δ 8.38 (d, J=8.0 Hz, 1H), 7.88 (d, J=7.2 Hz, 2H), 7.55 (t, J=7.6 Hz, 1H), 7.46 (t, J=7.6 Hz, 2H), 7.21 (s, 4H), 4.13 (d, J=2.4 Hz, 2H), 4.03-3.88 (m, 5H), 3.80-3.75 (m, 2H), 3.63-3.62 (m, 1H), 3.52 (t, J=6.4 Hz, 2H), 3.47 (bs, 2H), 3.45-3.39 (m, 2H), 3.28-3.26 (m, 1H), 2.82 (t, J=2.4 Hz, 1H), 2.77-2.73 (dd, J=4.8 Hz, 12.4 Hz, 1H), 1.78 (t, J=12.0 Hz, 1H), 1.58-1.55 (m, 4H) 1.39-1.38 (m, 4H).

### (2R,4S,5R,6R)-6-((1R,2R)-3-(2-(4-chlorophenyl)acetamido)-1,2-dihydroxypropyl)-5-(2,3-dihydroxypropanamido)-4-hydroxy-2-((6-(prop-2-yn-1-yloxy)hexyl)oxy)tetrahydro-2H-pyran-2-carboxylic acid (Compound 210)

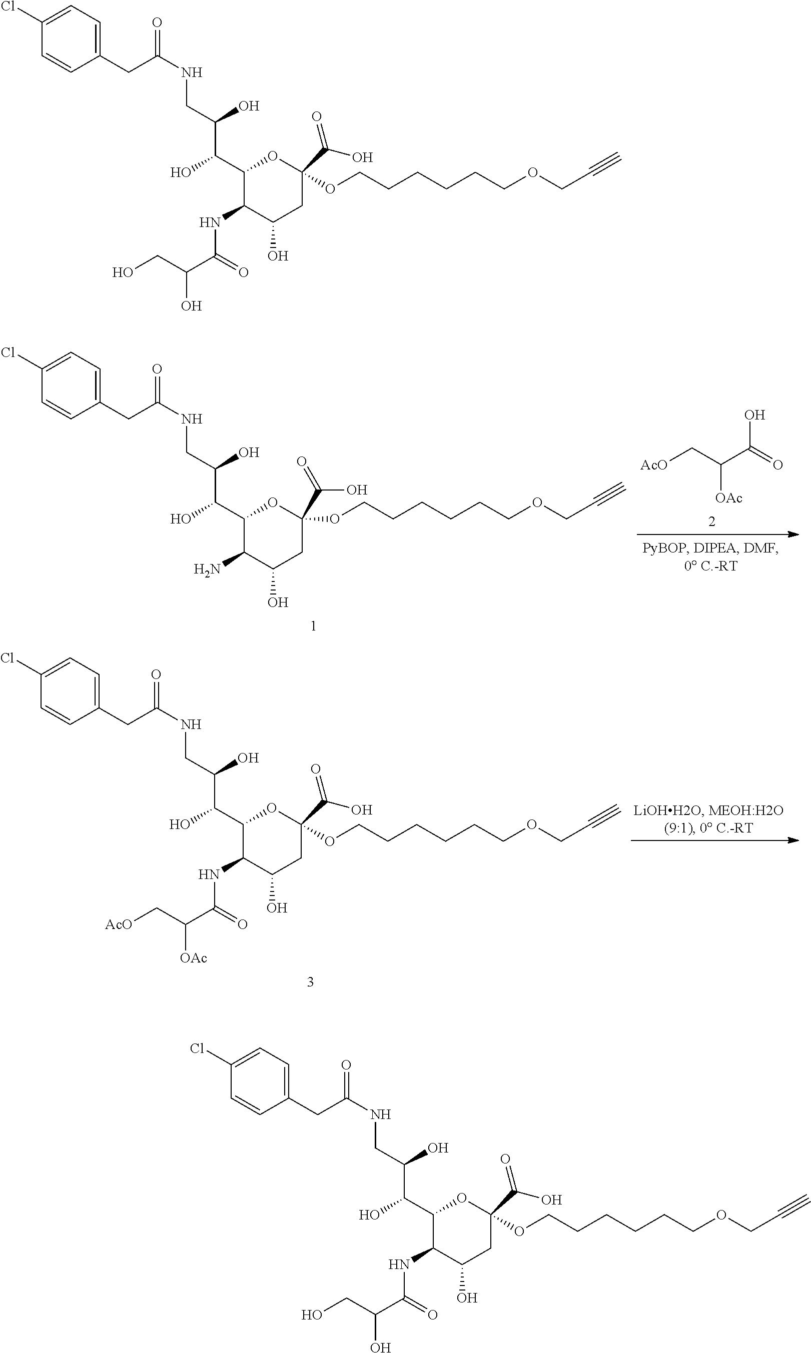

### Synthesis of (2R,4S,5R,6R)-6-((1R,2R)-3-(2-(4-chlorophenyl)acetamido)-1,2-dihydroxypropyl)-5-(2,3-diacetoxypropanamido)-4-hydroxy-2-((6-(prop-2-yn-1-yloxy)hexyl)oxy)tetrahydro-2H-pyran-2-carboxylic acid (3)

To a stirred solution of (2R,4S,5R,6R)-5-amino-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (1, 0.12 g, 0.22 mmol) and 2,3-diacetoxypropanoic acid (2, 0.06 g, 0.32 mmol) in N,N-dimethylformamide (3.0 mL) was added benzotriazol-1-yloxytripyrrolidinophosphonium hexafluorophosphate (0.22 g, 0.43 mmol) at 0° C. After 10 min, ethylbis(propan-2-yl)amine (0.18 mL, 1.08 mmol) was added dropwise was stirred at room temperature for 12 h. After completion, the reaction mixture was concentrated to afford the desired product (2R,4S,5R,6R)-6-((1R,2R)-3-(2-(4-chlorophenyl)acetamido)-1,2-dihydroxypropyl)-5-(2,3-diacetoxypropanamido)-4-hydroxy-2-((6-(prop-2-yn-1-yloxy)hexyl)oxy)tetrahydro-2H-pyran-2-carboxylic acid (3) as a crude which was used for next step without further purification. Yield: 0.08 g, 50.93%; LCMS m/z 728.70 [M+H]+;

### Synthesis of (2R,4S,5R,6R)-6-((1R,2R)-3-(2-(4-chlorophenyl)acetamido)-1,2-dihydroxypropyl)-5-(2,3-dihydroxypropanamido)-4-hydroxy-2-((6-(prop-2-yn-1-yloxy)hexyl)oxy)tetrahydro-2H-pyran-2-carboxylic acid (Cpd. 210)

To a stirred solution of (2R,4S,5R,6R)-6-((1R,2R)-3-(2-(4-chlorophenyl)acetamido)-1,2-dihydroxypropyl)-5-(2,3-diacetoxypropanamido)-4-hydroxy-2-((6-(prop-2-yn-1-yloxy)hexyl)oxy)tetrahydro-2H-pyran-2-carboxylic acid (3, 0.08 g, 0.11 mmol) in methanol (5.0 mL) was added a solution of lithium hydroxide monohydrate (0.005 g, 0.22 mmol) in water (0.5 mL). The reaction mixture was stirred at room temperature for 2 h. After completion, the reaction mixture was treated with acidic resin (Dowex 50, H⁺) to pH ˜6 and the suspension was filtered. The filtrate was concentrated, and the crude residue obtained was purified by Prep-HPLC using acetonitrile in water (+0.1% TFA) as eluents to afford the desired product (2R,4S,5R,6R)-6-((1R,2R)-3-(2-(4-chlorophenyl)acetamido)-1,2-dihydroxypropyl)-5-(2,3-dihydroxypropanamido)-4-hydroxy-2-((6-(prop-2-yn-1-yloxy)hexyl)oxy)tetrahydro-2H-pyran-2-carboxylic acid (Cpd. 210). Yield: 0.017 g, 24.02%; LCMS m/z 645.35 [M+H]+; ¹H-NMR (400 MHz, Methanol-d₄): δ 7.32-7.17 (m, 4H), 4.12 (d, J=2.4 Hz, 1H), 4.11-4.08 (m, 1H), 3.87-3.78 (m, 2H), 3.77-3.73 (m, 4H), 3.66-3.61 (m, 2H), 3.55-3.46 (m, 4H), 3.42-3.38 (m, 2H), 3.27-3.21 (m, 1H), 2.81 (t, J=2.4 Hz, 1H), 2.75-2.69 (m, 1H), 1.75-1.67 (m, 1H), 1.59-1.53 (m, 4H), 1.40-1.29 (m, 4H).

### (2R,4S,5R,6R)-6-((1R,2R)-3-(2-(4-chlorophenyl)acetamido)-1,2-dihydroxypropyl)-4-hydroxy-5-(2-(methylamino)acetamido)-2-((6-(prop-2-yn-1-yloxy)hexyl)oxy)tetrahydro-2H-pyran-2-carboxylic acid (Compound 211)

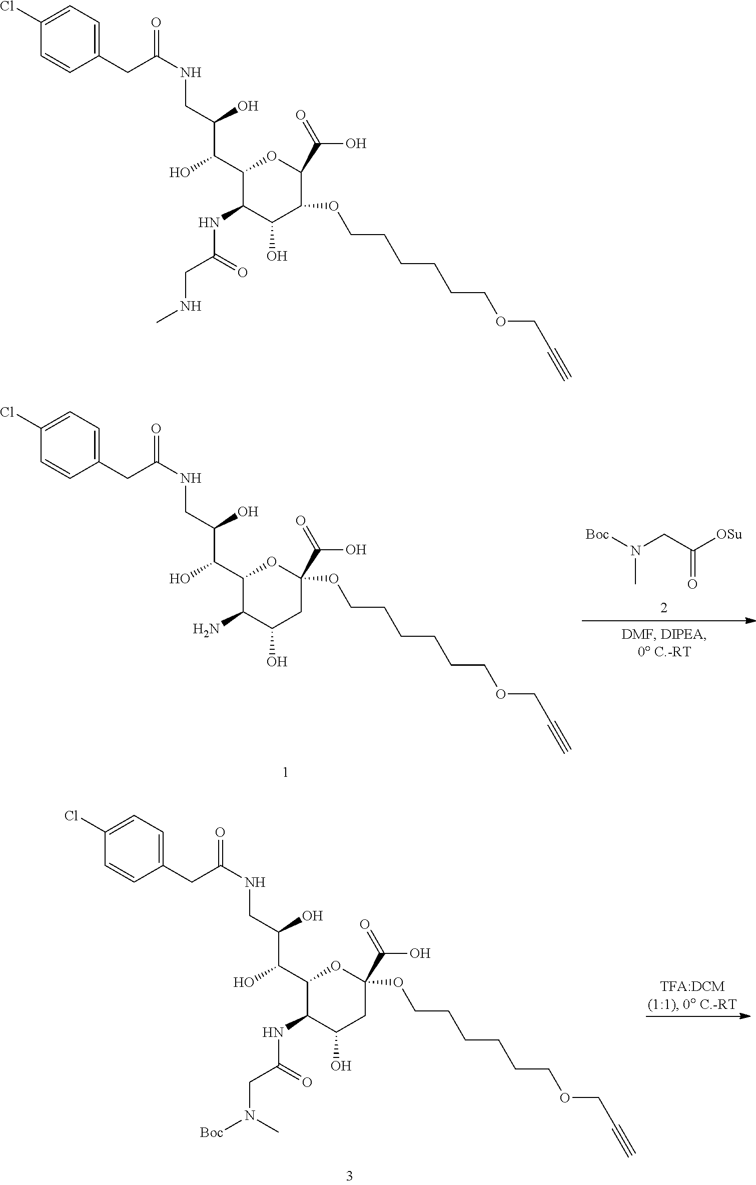

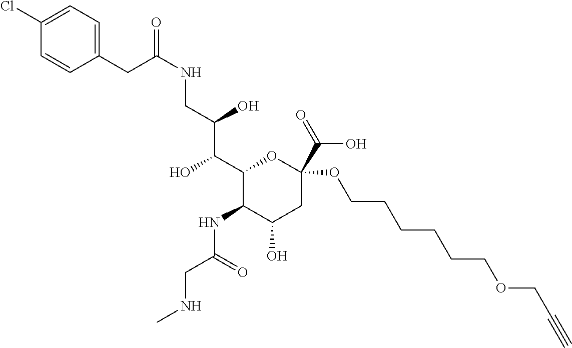 [SMILES: C#CCOCCCCCCO[C@]1(C(=O)O)C[C@H](O)[C@@H](NC(=O)CNC)[C@H]([C@H](O)[C@H](O)CNC(=O)Cc2ccc(Cl)cc2)O1]

### Synthesis of (2R,4S,5R,6R)-5-(2-((tert-butoxycarbonyl)(methyl)amino)acetamido)-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (3)

To a stirred solution of (2R,4S,5R,6R)-5-amino-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (1, 0.10 g, 0.18 mmol) in dry dimethylformamide (3.0 mL) were sequentially added 2,5-dioxopyrrolidin-1-yl N-(tert-butoxycarbonyl)-N-methylglycinate (0.051 g, 0.18 mmol) and N,N-Diisopropylethylamine (0.16 mL, 0.89 mmol) dropwise at 0° C. was stirred for 2 h at room temperature. After completion, the reaction mixture was concentrated to afford the desired product (2R,4S,5R,6R)-5-(2-((tert-butoxycarbonyl)(methyl)amino)acetamido)-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (3) as a crude which was used for the next step without further purification; Yield: 0.08 g, 61%; LCMS m/z 727.80 [M+H]+.

### Synthesis of (2R,4S,5R,6R)-6-((1R,2R)-3-(2-(4-chlorophenyl)acetamido)-1,2-dihydroxypropyl)-4-hydroxy-5-(2-(methylamino)acetamido)-2-((6-(prop-2-yn-1-yloxy)hexyl)oxy)tetrahydro-2H-pyran-2-carboxylic acid (Cpd. 211)

To a stirred solution of (2R,4S,5R,6R)-5-(2-((tert-butoxycarbonyl)(methyl)amino)acetamido)-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (3, 0.08 g, 0.11 mmol) in anhydrous dichloromethane (2.0 mL), trifluoroacetic acid (1.0 mL) was added at 0° C. then stirred at room temperature for 1 h. After completion the solvent was concentrated, and the crude residue obtained was purified by Prep-HPLC using acetonitrile in water (+0.1% TFA) as eluents to afford the desired product (2R,4S,5R,6R)-6-((1R,2R)-3-(2-(4-chlorophenyl)acetamido)-1,2-dihydroxypropyl)-4-hydroxy-5-(2-(methylamino)acetamido)-2-((6-(prop-2-yn-1-yloxy)hexyl)oxy)tetrahydro-2H-pyran-2-carboxylic acid (Cpd. 211). Yield: 0.049 g, 96%; LCMS m/z 628.40 [M+H]+; ¹H-NMR (400 MHz, Methanol-d₄): δ 7.33-7.28 (m, 4H), 4.12 (d, J=2.4 Hz, 2H), 3.87 (td, J=8.4 Hz & 2.8 Hz, 1H), 3.83-3.73 (m, 4H), 3.70-3.61 (m, 3H), 3.53-3.49 (m, 4H), 3.44-3.41 (m, 1H), 3.31 (d, J=1.6 Hz, 1H), 3.30-3.18 (m, 1H), 2.81 (t, J=2.4 Hz, 1H), 2.75 (dd, J=12.8 Hz & 4.8 Hz, 1H), 2.71 (s, 3H), 1.69 (t, J=12.4 Hz, 1H), 1.57-1.55 (m, 4H), 1.40-1.36 (m, 4H).

### (2R,4S,5R,6R)-6-((1R,2R)-3-(2-(4-chlorophenyl)acetamido)-1,2-dihydroxypropyl)-5-(2-(dimethylamino)acetamido)-4-hydroxy-2-((6-(prop-2-yn-1-yloxy)hexyl)oxy)tetrahydro-2H-pyran-2-carboxylic acid (Compound 25)

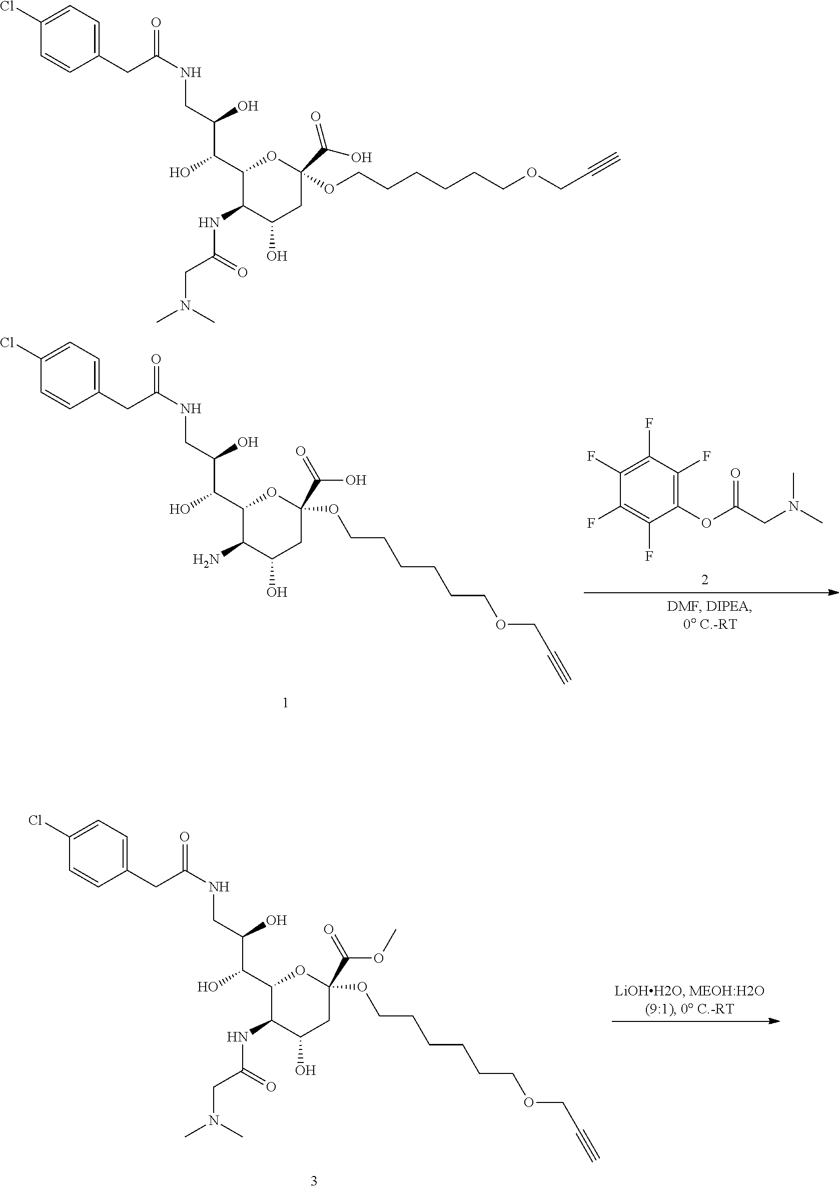

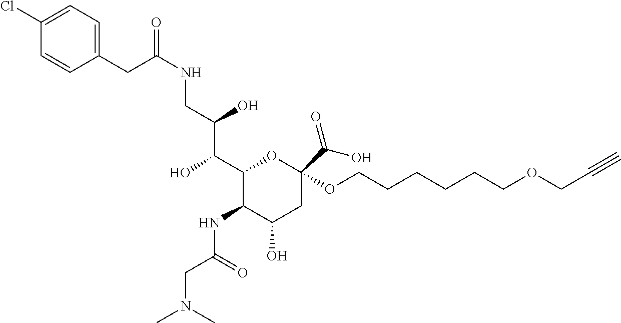 [SMILES: C#CCOCCCCCCO[C@]1(C(=O)O)C[C@H](O)[C@@H](NC(=O)CN(C)C)[C@H]([C@H](O)[C@H](O)CNC(=O)Cc2ccc(Cl)cc2)O1]

### Synthesis of methyl (2R,4S,5R,6R)-6-((1R,2R)-3-(2-(4-chlorophenyl)acetamido)-1,2-dihydroxypropyl)-5-(2-(dimethylamino)acetamido)-4-hydroxy-2-((6-(prop-2-yn-1-yloxy)hexyl)oxy)tetrahydro-2H-pyran-2-carboxylate (3)

To a stirred solution of methyl (2R,4S,5R,6R)-5-amino-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylate (1, 0.12 g, 0.21 mmol) in dry dimethylformamide (3.0 mL) were sequentially added perfluorophenyl dimethylglycinate (0.085 g, 0.31 mmol) and N,N-Diisopropylethylamine (0.18 mL, 1.05 mmol) dropwise was stirred for 2 h at room temperature. After completion, the reaction mixture was concentrated to afford the desired product methyl (2R,4S,5R,6R)-6-((1R,2R)-3-(2-(4-chlorophenyl)acetamido)-1,2-dihydroxypropyl)-5-(2-(dimethylamino)acetamido)-4-hydroxy-2-((6-(prop-2-yn-1-yloxy)hexyl)oxy)tetrahydro-2H-pyran-2-carboxylate (3) as a crude product which was used for next step without further purification; Yield: 0.11 g, 70%; LCMS m/z 655.95 [M+H]+.

### Synthesis of (2R,4S,5R,6R)-6-((1R,2R)-3-(2-(4-chlorophenyl)acetamido)-1,2-dihydroxypropyl)-5-(2-(dimethylamino)acetamido)-4-hydroxy-2-((6-(prop-2-yn-1-yloxy)hexyl)oxy)tetrahydro-2H-pyran-2-carboxylic acid (Cpd. 25)

To a stirred solution of methyl (2R,4S,5R,6R)-6-((1R,2R)-3-(2-(4-chlorophenyl)acetamido)-1,2-dihydroxypropyl)-5-(2-(dimethylamino)acetamido)-4-hydroxy-2-((6-(prop-2-yn-1-yloxy)hexyl)oxy)tetrahydro-2H-pyran-2-carboxylate (3, 0.11 g, 0.16 mmol) in methanol (5.0 mL) was added a solution of lithium hydroxide monohydrate (0.024 g, 0.96 mmol) in water (1.0 mL). The reaction mixture was stirred at room temperature for 5 h. After completion, the reaction mixture was treated with acidic resin (Dowex 50, H⁺) to pH ˜6 and the suspension was filtered. The filtrate was concentrated, and the crude residue obtained was purified by Prep-HPLC using acetonitrile in water (+0.1% TFA) as eluents to afford the desired product (2R,4S,5R,6R)-6-((1R,2R)-3-(2-(4-chlorophenyl)acetamido)-1,2-dihydroxypropyl)-5-(2-(dimethylamino)acetamido)-4-hydroxy-2-((6-(prop-2-yn-1-yloxy)hexyl)oxy)tetrahydro-2H-pyran-2-carboxylic acid (Cpd. 25). Yield: 0.078 g, 72%; LCMS m/z 642.40 [M+H]+; ¹H-NMR (400 MHz, Methanol-d₄): δ 7.34-7.29 (m, 4H), 4.13 (d, J=2.4 Hz, 2H), 3.96-3.86 (m, 4H), 3.79-3.60 (m, 4H), 3.55-3.51 (m, 4H), 3.46-3.40 (m, 1H), 3.36 (d, J=1.6 Hz, 1H), 3.28-3.22 (m, 1H), 2.94-2.92 (m, 6H), 2.82 (t, J=2.4 Hz, 1H), 2.77 (dd, J=12.8 Hz & 4.8 Hz, 1H), 1.72 (t, J=12.0 Hz, 1H), 1.60-1.55 (m, 4H), 1.39-1.36 (m, 4H).

### (2R,4S,5R,6R)-5-(2-aminoacetamido)-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (Compound 184)

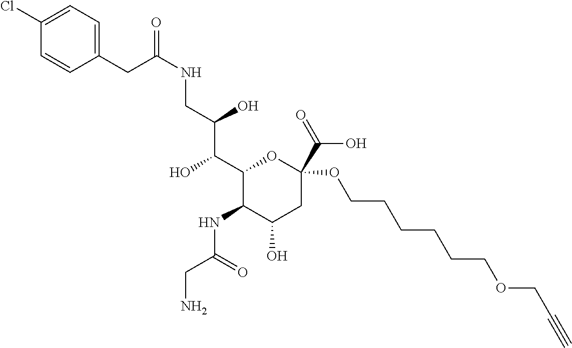 [SMILES: C#CCOCCCCCCO[C@]1(C(=O)O)C[C@H](O)[C@@H](NC(=O)CN)[C@H]([C@H](O)[C@H](O)CNC(=O)Cc2ccc(Cl)cc2)O1]

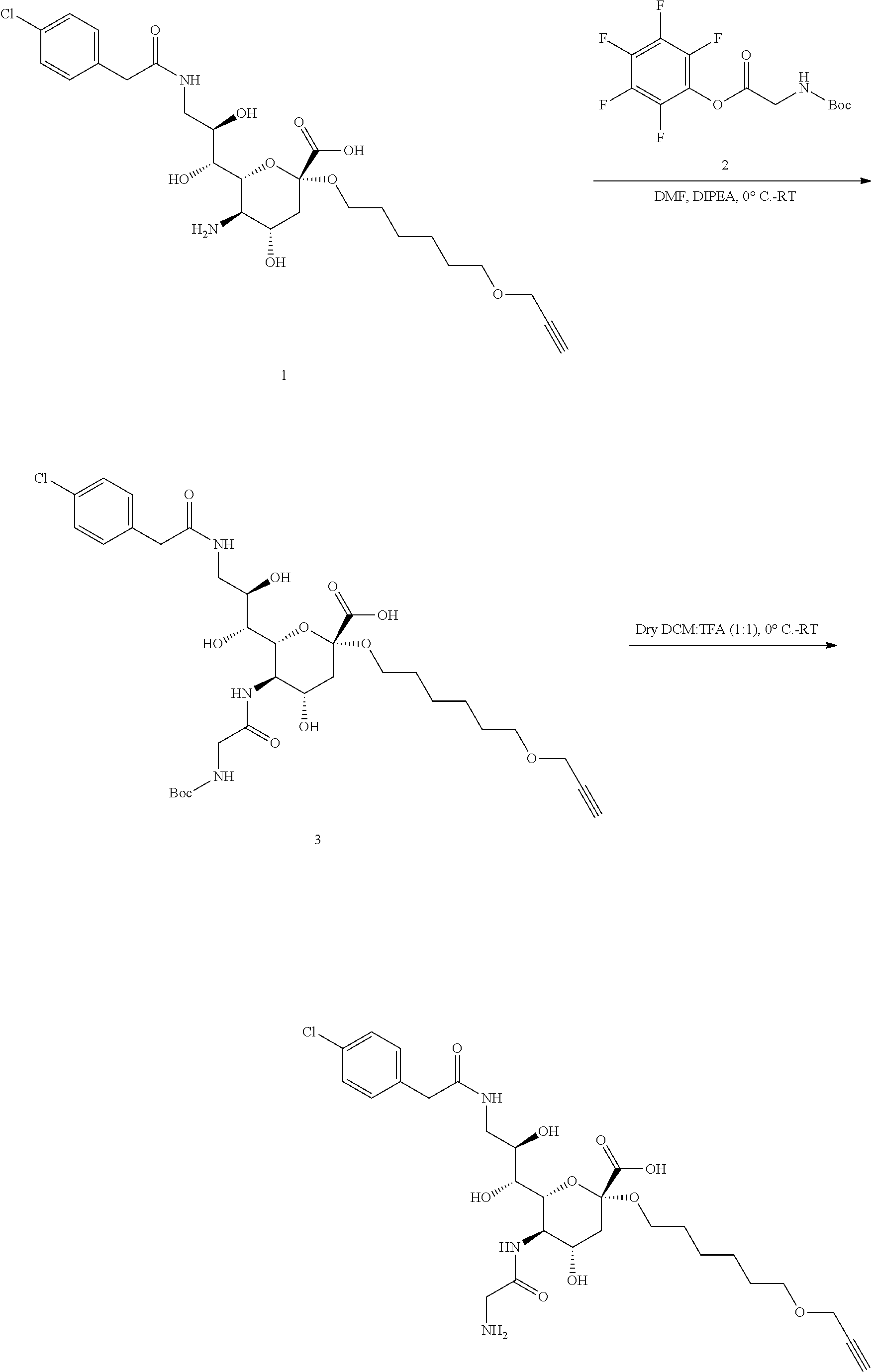 [SMILES: C#CCOCCCCCCO[C@]1(C(=O)O)C[C@H](O)[C@@H](N)[C@H]([C@H](O)[C@H](O)CNC(=O)Cc2ccc(Cl)cc2)O1 ; CC(C)(C)OC(=O)NCC(=O)Oc1c(F)c(F)c(F)c(F)c1F ; C#CCOCCCCCCO[C@]1(C(=O)O)C[C@H](O)[C@@H](NC(=O)CNC(=O)OC(C)(C)C)[C@H]([C@H](O)[C@H](O)CNC(=O)Cc2ccc(Cl)cc2)O1 ; C#CCOCCCCCCO[C@]1(C(=O)O)C[C@H](O)[C@@H](NC(=O)CN)[C@H]([C@H](O)[C@H](O)CNC(=O)Cc2ccc(Cl)cc2)O1]

### Synthesis of (2R,4S,5R,6R)-5-(2-((tert-butoxycarbonyl)amino)acetamido)-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (3)

To a stirred solution of (2R,4S,5R,6R)-5-amino-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (1, 0.15 g, 0.27 mmol) in N,N-dimethylformamide (3.0 mL) was added ethylbis(propan-2-yl)amine (0.25 mL, 1.35 mmol) and perfluorophenyl (tert-butoxycarbonyl)glycinate (2, 0.092 g, 0.27 mmol) at 0° C. then stirred at room temperature for 1 h. After completion, the reaction mixture was concentrated, and the crude residue obtained was purified by Prep-HPLC using acetonitrile in water (+0.1% TFA) as eluents to afford the desired product (2R,4S,5R,6R)-5-(2-((tert-butoxycarbonyl)amino)acetamido)-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (3). Yield: 0.11 g, 57%; LCMS m/z 714.45 [M+H]⁺;

### Synthesis of (2R,4S,5R,6R)-5-(2-aminoacetamido)-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (Cpd. 184)

To a stirred solution of (2R,4S,5R,6R)-5-(2-((tert-butoxycarbonyl)amino)acetamido)-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (3, 0.11 g, 0.15 mmol), in anhydrous dichloromethane (3.0 mL), trifluoroacetic acid (1.0 mL) was added at 0° C. then stirred at room temperature for 3 h. After completion the solvent was concentrated, and the crude residue obtained was purified by Prep-HPLC using acetonitrile in water (+0.1% TFA) as eluents to afford the desired product (2R,4S,5R,6R)-5-(2-aminoacetamido)-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (Cpd. 184). Yield: 0.077 g, 81%; LCMS m/z 614.40 [M+H]+; ¹H-NMR (400 MHz, Methanol-d4): δ 7.32-7.27 (m, 4H), 4.12 (d, J=2.4 Hz, 2H), 3.88 (td, J=8.4 Hz & 2.8 Hz, 1H), 3.79-3.73 (m, 2H), 3.69-3.60 (m, 5H), 3.53-3.47 (m, 4H), 3.46-3.40 (m, 1H), 3.35-3.34 (m, 1H), 3.21-3.17 (m, 1H), 2.82-2.76 (m, 2H), 1.65 (t, J=12.0 Hz, 1H), 1.58-1.53 (m, 4H), 1.38-1.36 (m, 4H).

### (2R,4S,5R,6R)-6-((1R,2R)-3-(2-(4-chlorophenyl)acetamido)-1,2-dihydroxypropyl)-4-hydroxy-5-(2-hydroxypropanamido)-2-((6-(prop-2-yn-1-yloxy)hexyl)oxy)tetrahydro-2H-pyran-2-carboxylic acid (Compound 183)

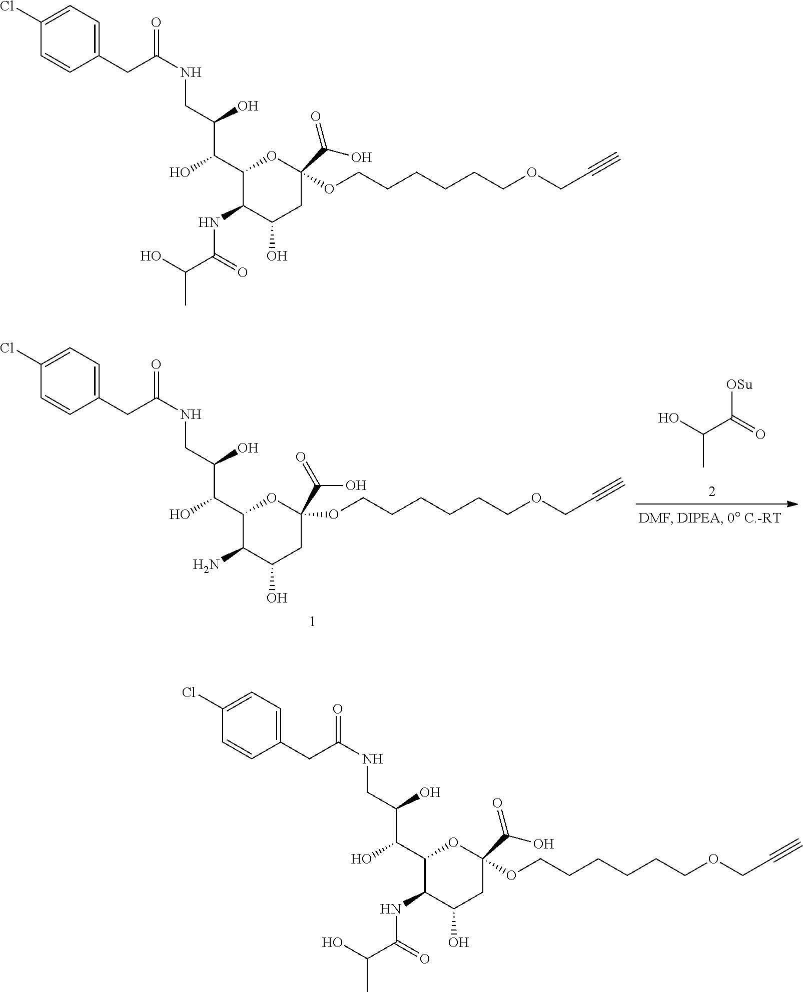

### Synthesis of (2R,4S,5R,6R)-6-((1R,2R)-3-(2-(4-chlorophenyl)acetamido)-1,2-dihydroxypropyl)-4-hydroxy-5-(2-hydroxypropanamido)-2-((6-(prop-2-yn-1-yloxy)hexyl)oxy)tetrahydro-2H-pyran-2-carboxylic acid (Cpd. 183)

To a stirred solution of (2R,4S,5R,6R)-5-amino-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (1, 0.10 g, 0.18 mmol) in N,N-dimethylformamide (3.0 mL) was added ethylbis(propan-2-yl)amine (0.15 mL, 0.89 mmol) and 2,5-dioxopyrrolidin-1-yl 2-hydroxypropanoate (2, 0.034 g, 0.18 mmol) at 0° C. then stirred at room temperature for 1 h. After completion, the reaction mixture was concentrated, and the crude residue obtained was purified by Prep-HPLC using acetonitrile in water (+0.1% TFA) as eluents to afford the desired product (2R,4S,5R,6R)-6-((1R,2R)-3-(2-(4-chlorophenyl)acetamido)-1,2-dihydroxypropyl)-4-hydroxy-5-(2-hydroxypropanamido)-2-((6-(prop-2-yn-1-yloxy)hexyl)oxy)tetrahydro-2H-pyran-2-carboxylic acid (Cpd. 183). Yield: 0.046 g, 41%; LCMS m/z 629.35 [M+H]+; ¹H-NMR (400 MHz, Methanol-d₄): δ 7.31-7.27 (m, 4H), 4.15-4.13 (m, 1H), 4.12 (d, J=2.4 Hz, 2H), 3.88 (td, J=8.4 Hz & 2.8 Hz, 1H), 3.82-3.71 (m, 3H), 3.67-3.60 (m, 2H), 3.53-3.48 (m, 4H), 3.42-3.37 (m, 1H), 3.34-3.33 (m, 1H), 3.30-3.24 (m, 1H), 2.80 (t, J=2.4 Hz, 1H), 2.71 (dd, J=12.8 Hz & 4.4 Hz, 1H), 1.71 (t, J=12.0 Hz, 1H), 1.57-1.53 (m, 4H), 1.40-1.29 (m, 7H).

### (2R,4S,5R,6R)-6-((1R,2R)-3-(2-(4-chlorophenyl)acetamido)-1,2-dihydroxypropyl)-4-hydroxy-5-(2-phenoxyacetamido)-2-((6-(prop-2-yn-1-yloxy)hexyl)oxy)tetrahydro-2H-pyran-2-carboxylic acid (Compound 91)

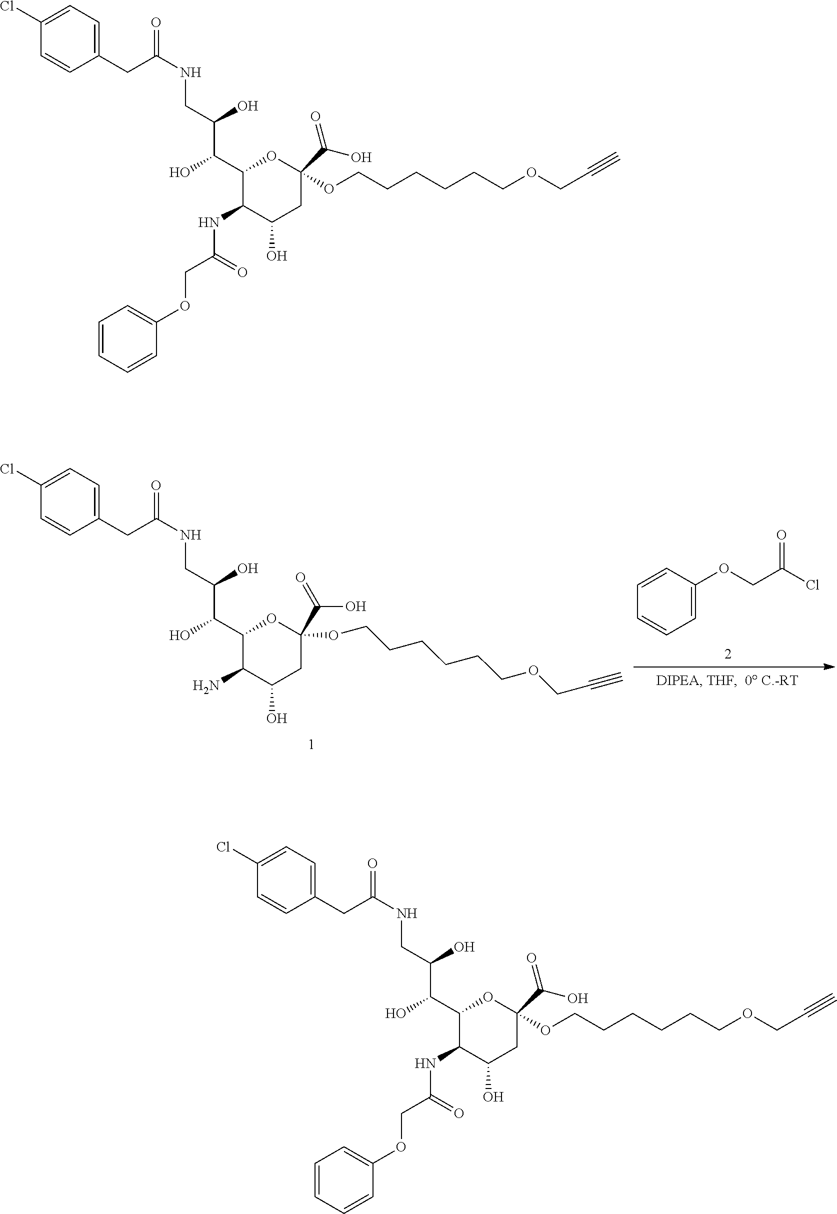 [SMILES: C#CCOCCCCCCO[C@]1(C(=O)O)C[C@H](O)[C@@H](NC(=O)COc2ccccc2)[C@H]([C@H](O)[C@H](O)CNC(=O)Cc2ccc(Cl)cc2)O1 ; C#CCOCCCCCCO[C@]1(C(=O)O)C[C@H](O)[C@@H](N)[C@H]([C@H](O)[C@H](O)CNC(=O)Cc2ccc(Cl)cc2)O1 ; O=C(Cl)COc1ccccc1 ; C#CCOCCCCCCO[C@]1(C(=O)O)C[C@H](O)[C@@H](NC(=O)COc2ccccc2)[C@H]([C@H](O)[C@H](O)CNC(=O)Cc2ccc(Cl)cc2)O1]

### Synthesis of (2R,4S,5R,6R)-6-((1R,2R)-3-(2-(4-chlorophenyl)acetamido)-1,2-dihydroxypropyl)-4-hydroxy-5-(2-phenoxyacetamido)-2-((6-(prop-2-yn-1-yloxy)hexyl)oxy)tetrahydro-2H-pyran-2-carboxylic acid (Cpd. 91)

To a stirred solution of (2R,4S,5R,6R)-5-amino-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (1, 0.10 g, 0.180 mmol) in THF (3.0 mL) was added ethylbis(propan-2-yl)amine (0.116 mL, 0.898 mmol) and phenoxyacetyl chloride (2, 0.031 g, 0.180 mmol) at 0° C. then stirred at room temperature for 1 h. After completion, the reaction mixture was concentrated, and the crude residue obtained was purified by prep HPLC using acetonitrile in water (+0.05% TFA) to afford the desired product (2R,4S,5R,6R)-6-((1R,2R)-3-(2-(4-chlorophenyl)acetamido)-1,2-dihydroxypropyl)-4-hydroxy-5-(2-phenoxyacetamido)-2-((6-(prop-2-yn-1-yloxy)hexyl)oxy)tetrahydro-2H-pyran-2-carboxylic acid (Cpd. 91). Yield: 0.033 g, 26.6%; LCMS m/z 691.50 [M+H]+; ¹H NMR (400 MHz, methanol-d4): δ 8.14-8.12 (m, 1H), 7.32-7.26 (m, 6H), 7.01-6.96 (m, 3H), 4.54-4.49 (m, 2H), 4.12 (d, J=2.4 Hz, 2H), 3.91-3.78 (m, 5H), 3.63-3.59 (m, 1H), 3.53-3.49 (m, 4H), 3.42-3.30 (m, 2H), 3.21-3.16 (m, 1H), 2.81 (t, J=2.4 Hz, 1H), 2.73-2.69 (dd, J=4.4 Hz, 12.0 Hz, 1H), 1.73 (t, J=11.6 Hz, 1H), 1.57-1.53 (m, 4H) 1.39-1.35 (m, 4H).

### (2R,4S,5R,6R)-6-((1R,2R)-3-(2-(4-chlorophenyl)acetamido)-1,2-dihydroxypropyl)-4-hydroxy-2-((6-(prop-2-yn-1-yloxy)hexyl)oxy)-5-(2,2,2-trifluoroacetamido)tetrahydro-2H-pyran-2-carboxylic acid (Compound 90)

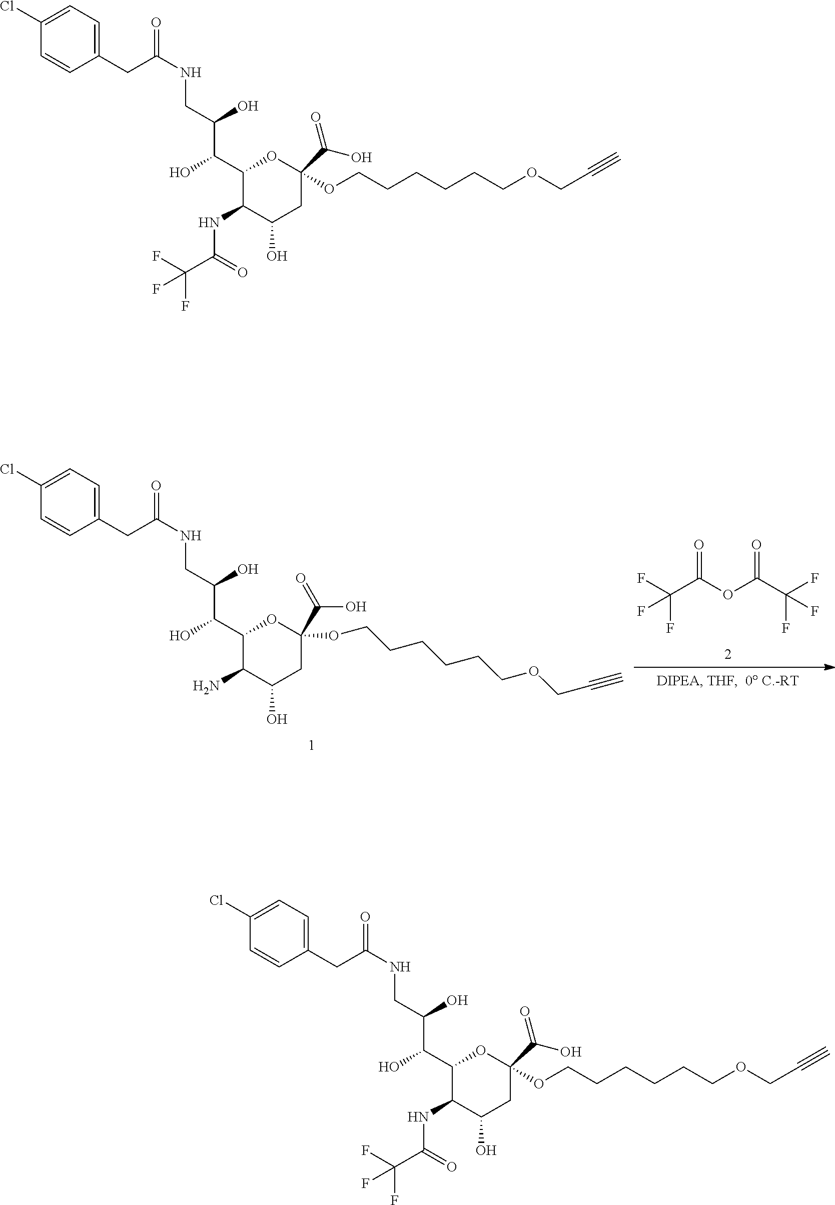 [SMILES: C#CCOCCCCCCO[C@]1(C(=O)O)C[C@H](O)[C@@H](NC(=O)C(F)(F)F)[C@H]([C@H](O)[C@H](O)CNC(=O)Cc2ccc(Cl)cc2)O1 ; C#CCOCCCCCCO[C@]1(C(=O)O)C[C@H](O)[C@@H](N)[C@H]([C@H](O)[C@H](O)CNC(=O)Cc2ccc(Cl)cc2)O1 ; O=C(OC(=O)C(F)(F)F)C(F)(F)F ; C#CCOCCCCCCO[C@]1(C(=O)O)C[C@H](O)[C@@H](NC(=O)C(F)(F)F)[C@H]([C@H](O)[C@H](O)CNC(=O)Cc2ccc(Cl)cc2)O1]

### Synthesis of (2R,4S,5R,6R)-6-((1R,2R)-3-(2-(4-chlorophenyl)acetamido)-1,2-dihydroxypropyl)-4-hydroxy-2-((6-(prop-2-yn-1-yloxy)hexyl)oxy)-5-(2,2,2-trifluoroacetamido)tetrahydro-2H-pyran-2-carboxylic acid (Cpd. 90)

To a stirred solution of (2R,4S,5R,6R)-5-amino-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (1, 0.10 g, 0.180 mmol) in THF (3.0 mL) was added ethylbis(propan-2-yl)amine (0.166 mL, 0.898 mmol) and 2,2,2-trifluoroacetyl trifluoroacetate (2, 0.038 g, 0.180 mmol) at 0° C. then stirred at room temperature for 1 h. After completion, the reaction mixture was concentrated, and the crude residue obtained was purified by prep HPLC using acetonitrile in water (+0.05% TFA) to afford the desired product (2R,4S,5R,6R)-6-((1R,2R)-3-(2-(4-chlorophenyl)acetamido)-1,2-dihydroxypropyl)-4-hydroxy-2-((6-(prop-2-yn-1-yloxy)hexyl)oxy)-5-(2,2,2-trifluoroacetamido)tetrahydro-2H-pyran-2-carboxylic acid (Cpd. 90). Yield: 0.018 g, 15.35%; LCMS m/z 653.3 [M+H]+; ¹H NMR (400 MHz, methanol-d4): 7.33-7.28 (m, 4H), 4.14 (d, J=2.4 Hz, 2H), 3.98-3.86 (m, 3H), 3.79-3.73 (m, 2H), 3.67-3.63 (m, 1H), 3.58-3.49 (m, 4H), 3.46-3.41 (m, 1H), 3.27-3.15 (m, 1H), 2.83 (t, J=2.4 Hz, 1H), 2.77-2.73 (dd, J=4.8 Hz, 12.8 Hz, 1H), 1.71 (t, J=12.0 Hz, 1H), 1.59-1.54 (m, 4H), 1.42-1.38 (m, 4H).

### (2R,4S,5R,6R)-6-((1R,2R)-3-(2-(4-chlorophenyl)acetamido)-1,2-dihydroxypropyl)-4-hydroxy-2-((6-(prop-2-yn-1-yloxy)hexyl)oxy)-5-(3-(2,2,2-trifluoroethyl)ureido)tetrahydro-2H-pyran-2-carboxylic acid (Compound 193)

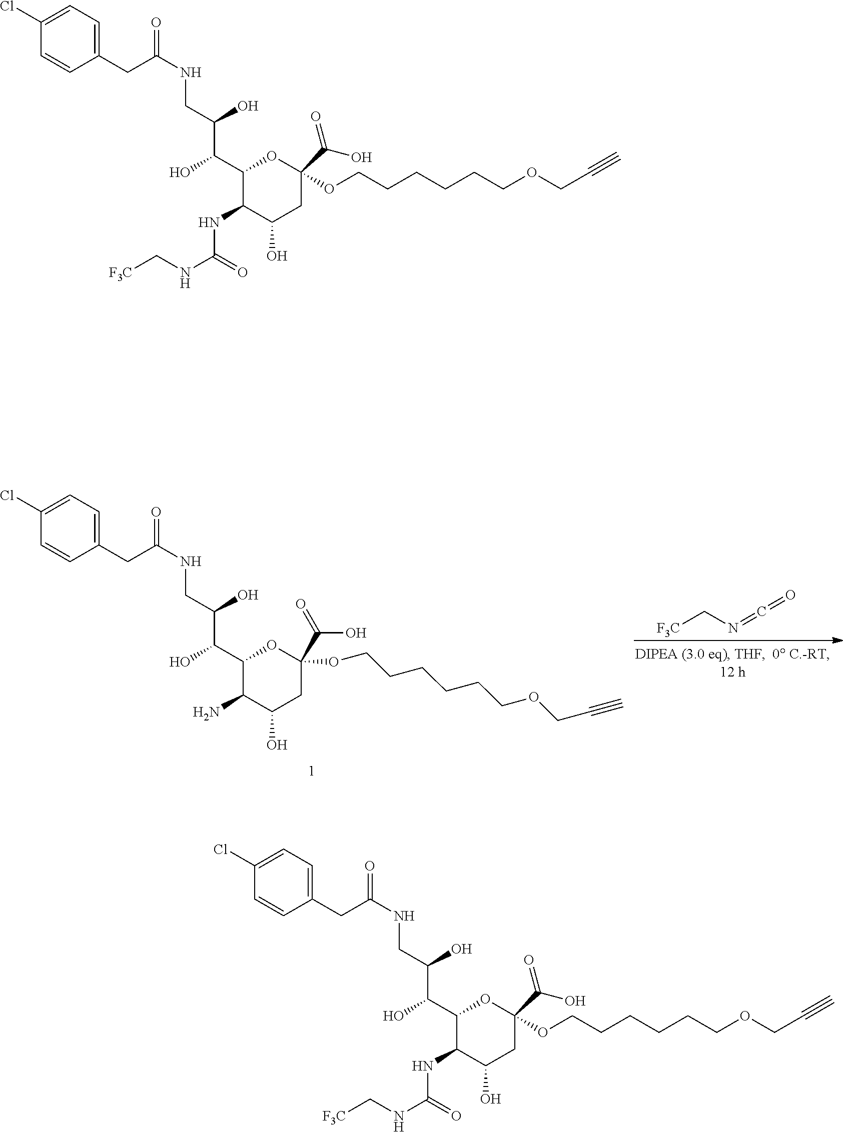 [SMILES: C#CCOCCCCCCO[C@]1(C(=O)O)C[C@H](O)[C@@H](NC(=O)NCC(F)(F)F)[C@H]([C@H](O)[C@H](O)CNC(=O)Cc2ccc(Cl)cc2)O1 ; C#CCOCCCCCCO[C@]1(C(=O)O)C[C@H](O)[C@@H](N)[C@H]([C@H](O)[C@H](O)CNC(=O)Cc2ccc(Cl)cc2)O1 ; O=C=NCC(F)(F)F ; C#CCOCCCCCCO[C@]1(C(=O)O)C[C@H](O)[C@@H](NC(=O)NCC(F)(F)F)[C@H]([C@H](O)[C@H](O)CNC(=O)Cc2ccc(Cl)cc2)O1]

### Synthesis of (2R,4S,5R,6R)-6-((1R,2R)-3-(2-(4-chlorophenyl)acetamido)-1,2-dihydroxypropyl)-4-hydroxy-2-((6-(prop-2-yn-1-yloxy)hexyl)oxy)-5-(3-(2,2,2-trifluoroethyl)ureido)tetrahydro-2H-pyran-2-carboxylic acid (Cpd. 193)

To a stirred solution of (2R,4S,5R,6R)-5-amino-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (1, 0.1 g, 0.19 mmol) in dry tetrahydrofuran (THF, 2.0 mL) were sequentially added 1,1,1-trifluoro-2-isocyanatoethane (0.03 g, 0.23 mmol) and Triethylamine (0.08 mL, 0.57 mmol) at 0° C. was stirred for 12 h at room temperature. After completion, the reaction mixture was concentrated. The crude residue obtained was purified by Prep-HPLC using acetonitrile in water (+0.1% TFA) as eluents to afford the desired product (2R,4S,5R,6R)-6-((1R,2R)-3-(2-(4-chlorophenyl)acetamido)-1,2-dihydroxypropyl)-4-hydroxy-2-((6-(prop-2-yn-1-yloxy)hexyl)oxy)-5-(3-(2,2,2-trifluoroethyl)ureido)tetrahydro-2H-pyran-2-carboxylic acid (Cpd. 193). Yield: 0.045 g, 36%; LCMS m/z 682.40 [M+H]+; ¹H-NMR (400 MHz, Methanol-d₄): δ 7.31-7.26 (m, 4H), 4.12 (d, J=2.4 Hz, 2H), 3.89-3.73 (m, 4H), 3.66-3.49 (m, 8H), 3.44-3.42 (m, 1H), 3.40-3.36 (m, 1H), 3.25 (dd, J=14.0 Hz & 8.0 Hz, 1H), 2.81 (t, J=2.4 Hz, 1H), 2.70 (dd, J=12.8 Hz & 4.0 Hz, 1H), 1.72 (t, J=12.4 Hz, 1H), 1.59-1.51 (m, 4H), 1.38-1.35 (m, 4H).

### (2R,4S,5R,6R)-6-((1R,2R)-3-(2-(4-chlorophenyl)acetamido)-1,2-dihydroxypropyl)-5-(3-cyclopropylureido)-4-hydroxy-2-((6-(prop-2-yn-1-yloxy)hexyl)oxy)tetrahydro-2H-pyran-2-carboxylic acid (Compound 89)

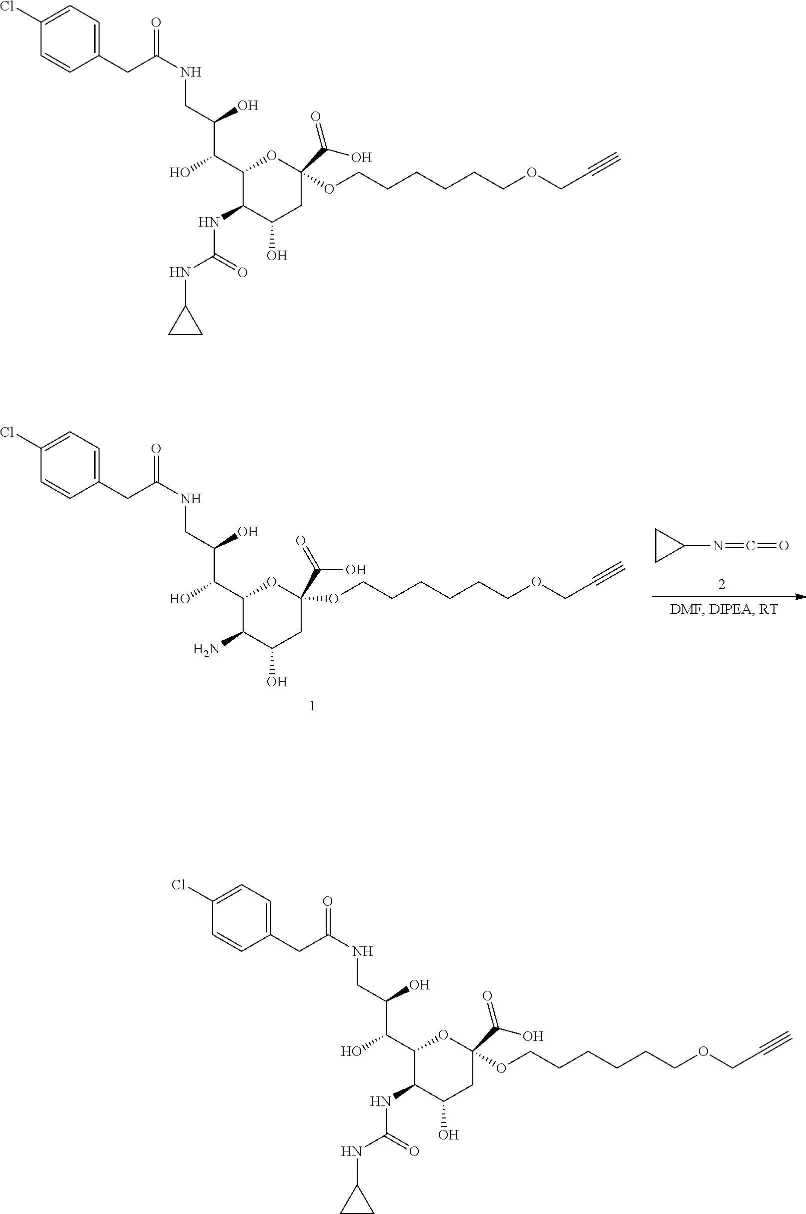

### Synthesis of (2R,4S,5R,6R)-6-((1R,2R)-3-(2-(4-chlorophenyl)acetamido)-1,2-dihydroxypropyl)-5-(3-cyclopropylureido)-4-hydroxy-2-((6-(prop-2-yn-1-yloxy)hexyl)oxy)tetrahydro-2H-pyran-2-carboxylic acid (Cpd. 89)

To a stirred solution of (2R,4S,5R,6R)-5-amino-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (1, 0.08 g, 0.14 mmol) in dry dichloromethane (1.0 mL) were sequentially added isocyanatocyclopropane (0.018 g, 0.21 mmol) and N,N-Diisopropylethylamine (0.12 mL, 0.67 mmol) dropwise was stirred for 12 h at room temperature. After completion, the reaction mixture was concentrated. The crude residue obtained was purified by Prep-HPLC using acetonitrile in water (+0.1% TFA) as eluents to afford the desired product (2R,4S,5R,6R)-6-((1R,2R)-3-(2-(4-chlorophenyl)acetamido)-1,2-dihydroxypropyl)-5-(3-cyclopropylureido)-4-hydroxy-2-((6-(prop-2-yn-1-yloxy)hexyl)oxy)tetrahydro-2H-pyran-2-carboxylic acid (Cpd. 89). Yield: 0.050 g, 54%; LCMS m/z 640.45 [M+H]+; ¹H-NMR (400 MHz, Methanol-d₄): δ 7.29 (m, 4H), 4.11 (d, J=2.4 Hz, 2H), 3.89 (td, J=8.0 Hz & 2.8 Hz, 1H), 3.77-3.72 (m, 1H), 3.67-3.63 (m, 2H), 3.58-3.55 (m, 1H), 3.54-3.49 (m, 5H), 3.45-3.37 (m, 2H), 3.28-3.23 (m, 1H), 2.82 (t, J=3.0 Hz, 1H), 2.72 (dd, J=12.8 Hz & 4.8 Hz, 1H), 2.48-2.43 (m, 1H), 1.72 (t, J=12.4 Hz, 1H), 1.57-1.53 (m, 4H), 1.40-1.36 (m, 4H), 0.70-0.67 (m, 2H), 0.51-0.44 (m, 2H).

### (2R,4S,5R,6R)-6-((1R,2R)-3-(2-(4-chlorophenyl)acetamido)-1,2-dihydroxypropyl)-5-(2-cyclopropylacetamido)-4-hydroxy-2-((6-(prop-2-yn-1-yloxy)hexyl)oxy)tetrahydro-2H-pyran-2-carboxylic acid (Compound 88)

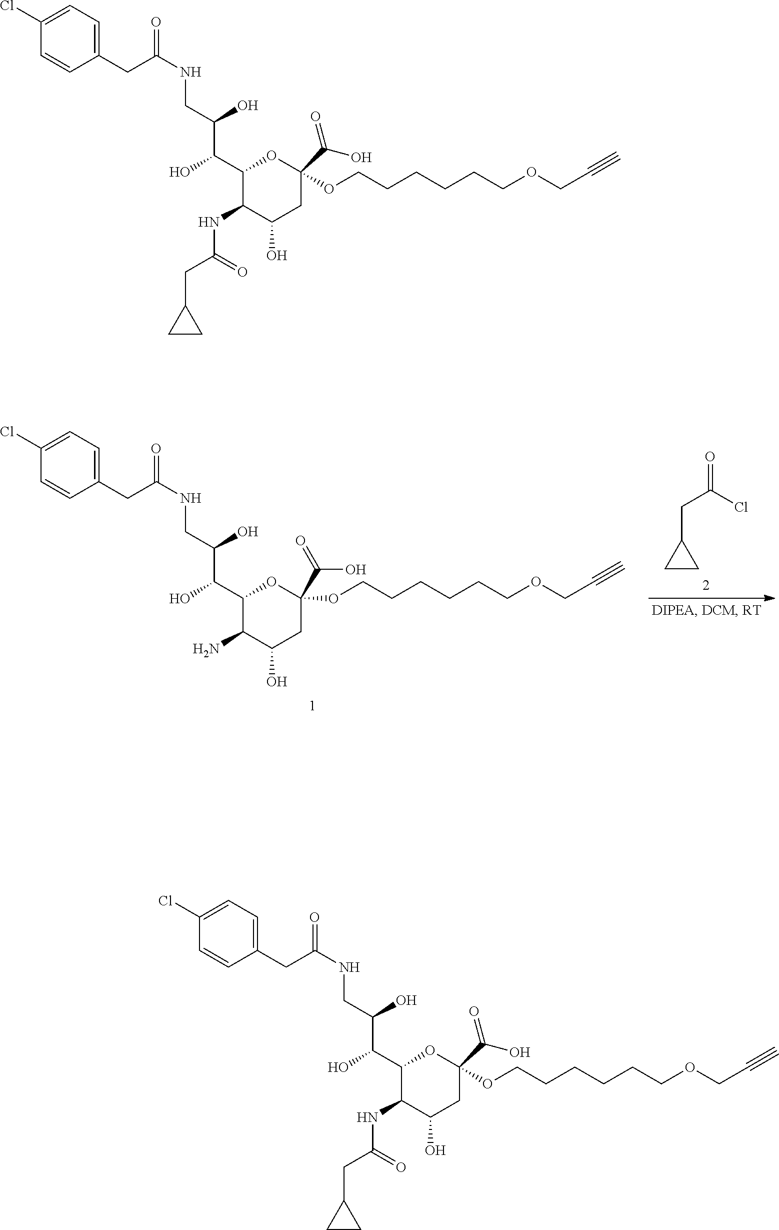

### Synthesis of (2R,4S,5R,6R)-6-((1R,2R)-3-(2-(4-chlorophenyl)acetamido)-1,2-dihydroxypropyl)-5-(2-cyclopropylacetamido)-4-hydroxy-2-((6-(prop-2-yn-1-yloxy)hexyl)oxy)tetrahydro-2H-pyran-2-carboxylic acid (Cpd. 88)

To a stirred solution of (2R,4S,5R,6R)-5-amino-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (1, 0.08 g, 0.14 mmol) in dry dichloromethane (1.0 mL) trimethylamine (0.06 mL, 0.42 mmol) was added dropwise at 0° C. After 10 min, 2-cyclopropylacetyl chloride (0.017 mL, 0.17 mmol) was added to the reaction mixture at that temperature and stirred for another 2 h at room temperature. After completion, the reaction mixture was concentrated. The crude residue obtained was purified by Prep-HPLC using acetonitrile in water (+0.1% TFA) as eluents to afford the desired product (2R,4S,5R,6R)-6-((1R,2R)-3-(2-(4-chlorophenyl)acetamido)-1,2-dihydroxypropyl)-5-(2-cyclopropylacetamido)-4-hydroxy-2-((6-(prop-2-yn-1-yloxy)hexyl)oxy)tetrahydro-2H-pyran-2-carboxylic acid (Cpd. 88). Yield: 0.032 g, 35%; LCMS m/z 639.45 [M+H]+; ¹H-NMR (400 MHz, Methanol-d₄): δ 7.92 (d, J=7.2 Hz, 1H), 7.31-7.27 (m, 4H), 4.11 (d, J=2.4 Hz, 2H), 3.89 (td, J=8.0 Hz & 2.8 Hz, 1H), 3.78-3.71 (m, 3H), 3.65-3.63 (d, J=13.6 Hz & 2.8 Hz, 1H), 3.59-3.57 (m, 1H), 3.54-3.49 (m, 4H), 3.43-3.37 (m, 2H), 3.31-3.26 (m, 1H), 2.81 (t, J=3.0 Hz, 1H), 2.72 (dd, J=12.8 Hz & 4.8 Hz, 1H), 2.19-2.04 (m, 2H), 1.72 (t, J=12.4 Hz, 1H), 1.57-1.53 (m, 4H), 1.40-1.36 (m, 4H), 1.06-0.98 (m, 1H), 0.55-0.49 (m, 2H), 0.23-0.17 (m, 2H).

### (2R,4S,5R,6R)-6-((1R,2R)-3-(2-(4-chlorophenyl)acetamido)-1,2-dihydroxypropyl)-5-(3-ethylureido)-4-hydroxy-2-((6-(prop-2-yn-1-yloxy)hexyl)oxy)tetrahydro-2H-pyran-2-carboxylic acid (Compound 87)

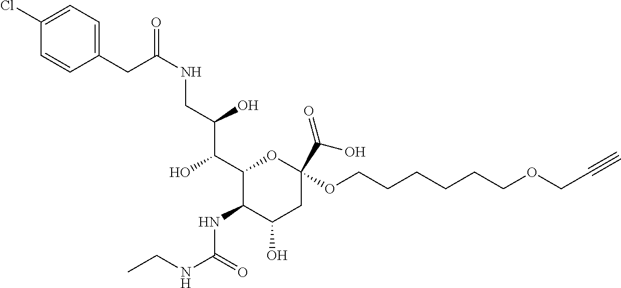 [SMILES: C#CCOCCCCCCO[C@]1(C(=O)O)C[C@H](O)[C@@H](NC(=O)NCC)[C@H]([C@H](O)[C@H](O)CNC(=O)Cc2ccc(Cl)cc2)O1]

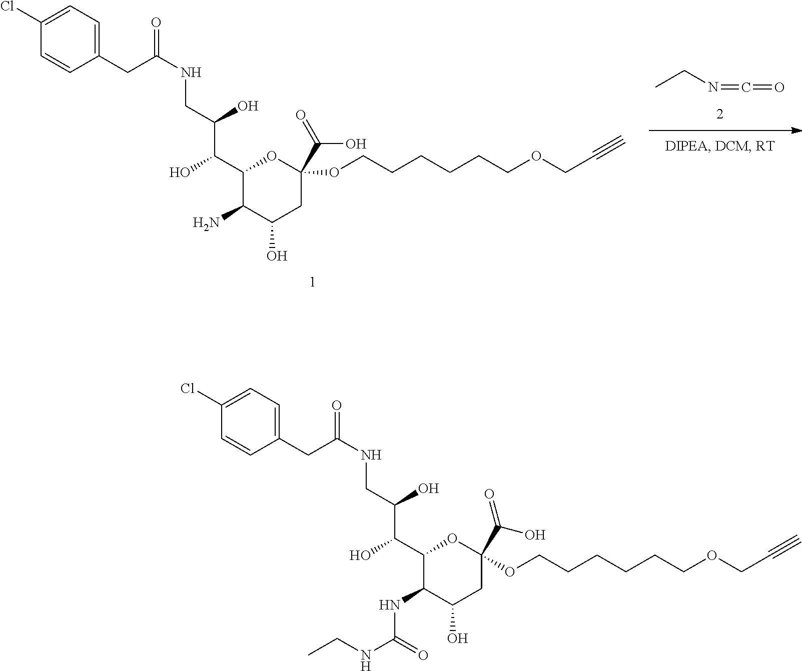

### Synthesis of (2R,4S,5R,6R)-6-((1R,2R)-3-(2-(4-chlorophenyl)acetamido)-1,2-dihydroxypropyl)-5-(3-ethylureido)-4-hydroxy-2-((6-(prop-2-yn-1-yloxy)hexyl)oxy)tetrahydro-2H-pyran-2-carboxylic acid (Cpd. 87)

To a stirred solution of (2R,4S,5R,6R)-5-amino-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (1, 0.08 g, 0.14 mmol) in dry dichloromethane (1.0 mL) were sequentially added isocyanatoethane (0.015 g, 0.21 mmol) and N,N-Diisopropylethylamine (0.12 mL, 0.72 mmol) dropwise was stirred for 12 h at room temperature. After completion, the reaction mixture was concentrated. The crude residue obtained was purified by Prep-HPLC using acetonitrile in water (+0.1% TFA) as eluents to afford the desired product (2R,4S,5R,6R)-6-((1R,2R)-3-(2-(4-chlorophenyl)acetamido)-1,2-dihydroxypropyl)-5-(3-ethylureido)-4-hydroxy-2-((6-(prop-2-yn-1-yloxy)hexyl)oxy)tetrahydro-2H-pyran-2-carboxylic acid (Cpd. 87). Yield: 0.04 g, 44%; LCMS m/z 628.45 [M+H]+; ¹H-NMR (400 MHz, Methanol-d₄): δ 7.31-7.27 (m, 4H), 4.11 (d, J=2.4 Hz, 2H), 3.89 (td, J=8.0 Hz & 2.8 Hz, 1H), 3.77-3.72 (m, 1H), 3.65-3.62 (m, 1H), 3.59-3.46 (m, 8H), 3.41-3.36 (m, 1H), 3.28-3.24 (m, 1H), 3.17-3.11 (m, 2H), 2.81 (t, J=2.4 Hz, 1H), 2.72 (dd, J=12.8 Hz & 4.8 Hz, 1H), 1.72 (t, J=12.0 Hz, 1H), 1.57-1.53 (m, 4H), 1.38-1.36 (m, 4H), 1.09 (t, J=7.2 Hz, 3H).

### (2R,4S,5R,6R)-6-((1R,2R)-3-(2-(4-chlorophenyl)acetamido)-1,2-dihydroxypropyl)-4-hydroxy-2-((6-(prop-2-yn-1-yloxy)hexyl)oxy)-5-ureidotetrahydro-2H-pyran-2-carboxylic acid (Compound 86)

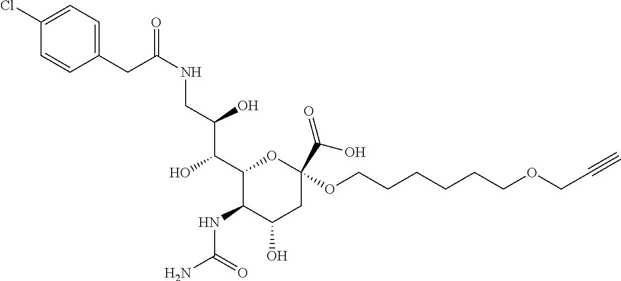 [SMILES: C#CCOCCCCCCO[C@]1(C(=O)O)C[C@H](O)[C@@H](NC(N)=O)[C@H]([C@H](O)[C@H](O)CNC(=O)Cc2ccc(Cl)cc2)O1]

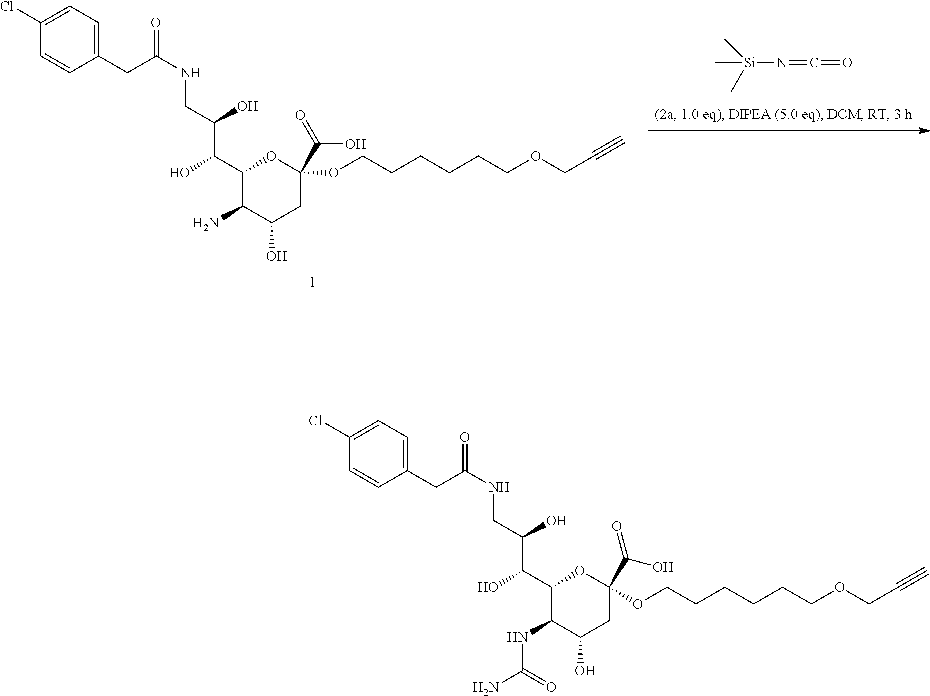

### Synthesis of (2R,4S,5R,6R)-6-((1R,2R)-3-(2-(4-chlorophenyl)acetamido)-1,2-dihydroxypropyl)-4-hydroxy-2-((6-(prop-2-yn-1-yloxy)hexyl)oxy)-5-ureidotetrahydro-2H-pyran-2-carboxylic acid (Cpd. 86)

To a stirred solution of (2R,4S,5R,6R)-5-amino-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (0.060 g, 0.108 mmol) in anhydrous dichloromethane (2 mL) was added isocyanatotrimethylsilane (0.018 g, 0.162 mmol) in dichloromethane (0.1 mL) at 0° C. followed by addition of N-ethylbis(isopropyl)amine (0.056 mL, 0.323 mmol) was stirred at room temperature for 2 h. After completion, the reaction mixture was concentrated. The crude residue obtained was purified by Prep-HPLC using acetonitrile in water (+0.1% TFA) as eluents to afford the desired product (2R,4S,5R,6R)-6-((1R,2R)-3-(2-(4-chlorophenyl)acetamido)-1,2-dihydroxypropyl)-4-hydroxy-2-((6-(prop-2-yn-1-yloxy)hexyl)oxy)-5-ureidotetrahydro-2H-pyran-2-carboxylic acid (Cpd. 86). Yield: 0.021 g, 33%; LCMS m/z 600.35 [M+H]+; ¹H-NMR (400 MHz, Methanol-d4): δ 8.05-8.02 (m, 1H), 7.31-7.27 (m, 4H), 4.12 (d, J=2.4 Hz, 2H), 3.91-3.86 (m, 1H), 3.77-3.72 (m, 1H), 3.67-3.64 (m, 1H), 3.62-3.46 (m, 8H), 3.43-3.37 (m, 1H), 3.30-3.24 (m, 1H), 2.81 (t, J=2.4 Hz, 1H), 2.73 (dd, J=12.6 Hz & 4.4 Hz, 1H), 1.68 (t, J=12.0 Hz, 1H), 1.57-1.53 (m, 4H), 1.38-1.35 (m, 4H).

### (2R,4S,5R,6R)-6-((1R,2R)-3-(2-(4-chlorophenyl)acetamido)-1,2-dihydroxypropyl)-4-hydroxy-5-((2-(methylamino)-3,4-dioxocyclobut-1-en-1-yl)amino)-2-((6-(prop-2-yn-1-yloxy)hexyl)oxy)tetrahydro-2H-pyran-2-carboxylic acid (Compound 191)

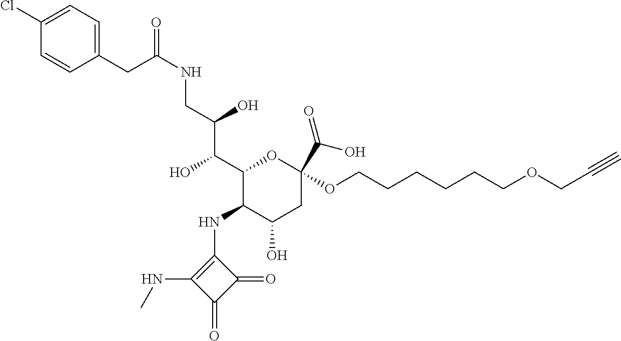 [SMILES: C#CCOCCCCCCO[C@]1(C(=O)O)C[C@H](O)[C@@H](Nc2c(NC)c(=O)c2=O)[C@H]([C@H](O)[C@H](O)CNC(=O)Cc2ccc(Cl)cc2)O1]

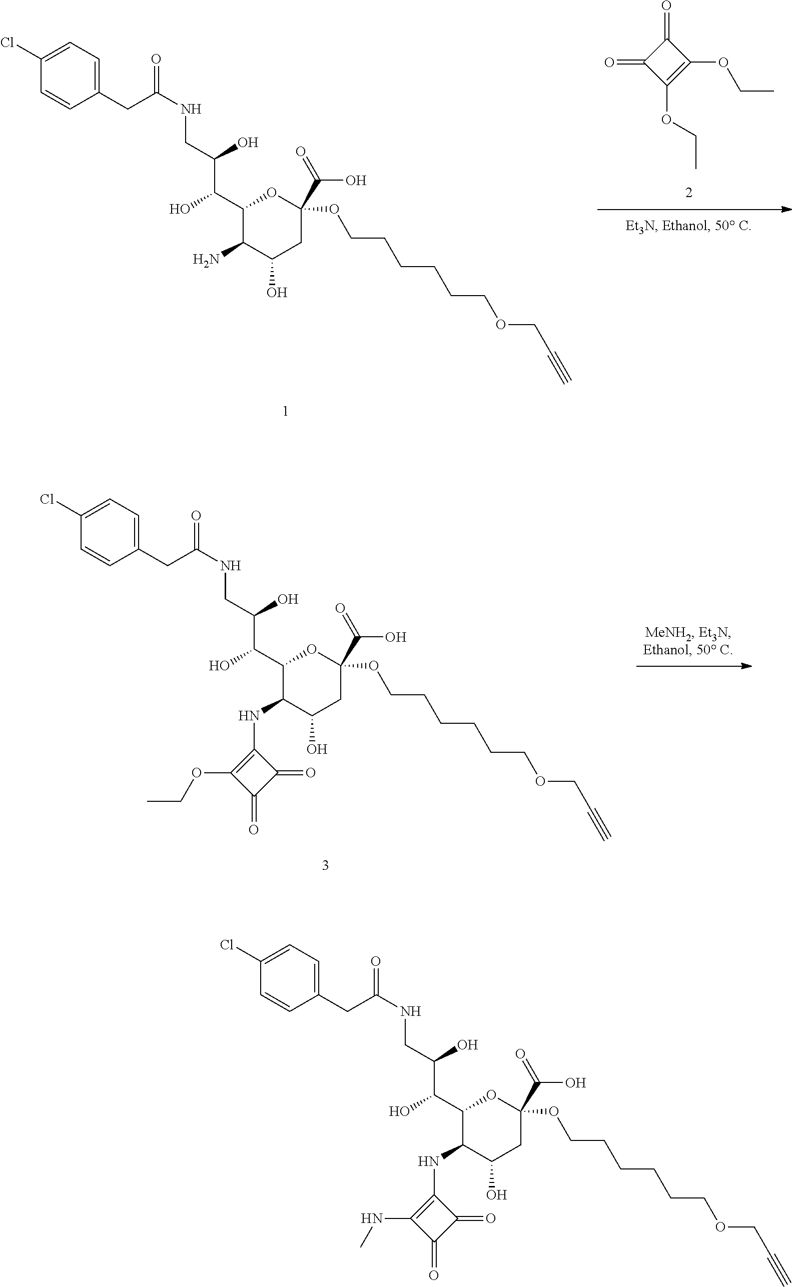 [SMILES: C#CCOCCCCCCO[C@]1(C(=O)O)C[C@H](O)[C@@H](N)[C@H]([C@H](O)[C@H](O)CNC(=O)Cc2ccc(Cl)cc2)O1 ; CCOc1c(OCC)c(=O)c1=O ; C#CCOCCCCCCO[C@]1(C(=O)O)C[C@H](O)[C@@H](Nc2c(OCC)c(=O)c2=O)[C@H]([C@H](O)[C@H](O)CNC(=O)Cc2ccc(Cl)cc2)O1 ; C#CCOCCCCCCO[C@]1(C(=O)O)C[C@H](O)[C@@H](Nc2c(NC)c(=O)c2=O)[C@H]([C@H](O)[C@H](O)CNC(=O)Cc2ccc(Cl)cc2)O1]

### Synthesis of (2R,4S,5R,6R)-6-((1R,2R)-3-(2-(4-chlorophenyl)acetamido)-1,2-dihydroxypropyl)-5-((2-ethoxy-3,4-dioxocyclobut-1-en-1-yl)amino)-4-hydroxy-2-((6-(prop-2-yn-1-yloxy)hexyl)oxy)tetrahydro-2H-pyran-2-carboxylic acid (3)

To a stirred solution of (2R,4S,5R,6R)-5-amino-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (1, 0.050 g, 0.09 mmol) in 3,4-diethoxycyclobut-3-ene-1,2-dione (2, 0.038 g, 0.22 mmol)_ethanol (2 mL), was added triethylamine (0.037 mL, 0.27 mmol) at room temperature was stirred at 50° C. for 12 h. After completion, the reaction mixture was concentrated to afford the desired product (2R,4S,5R,6R)-6-((1R,2R)-3-(2-(4-chlorophenyl)acetamido)-1,2-dihydroxypropyl)-5-((2-ethoxy-3,4-dioxocyclobut-1-en-1-yl)amino)-4-hydroxy-2-((6-(prop-2-yn-1-yloxy)hexyl)oxy)tetrahydro-2H-pyran-2-carboxylic acid (3) as a crude which was used for the next step without further purification. Yield: 0.070 g, Crude; LCMS m/z 682.90 [M+H]⁺.

### Synthesis of (2R,4S,5R,6R)-6-((1R,2R)-3-(2-(4-chlorophenyl)acetamido)-1,2-dihydroxypropyl)-4-hydroxy-5-((2-(methylamino)-3,4-dioxocyclobut-1-en-1-yl)amino)-2-((6-(prop-2-yn-1-yloxy)hexyl)oxy)tetrahydro-2H-pyran-2-carboxylic acid (Cpd. 191)

To a stirred solution of (2R,4S,5R,6R)-6-((1R,2R)-3-(2-(4-chlorophenyl)acetamido)-1,2-dihydroxypropyl)-5-((2-ethoxy-3,4-dioxocyclobut-1-en-1-yl)amino)-4-hydroxy-2-((6-(prop-2-yn-1-yloxy)hexyl)oxy)tetrahydro-2H-pyran-2-carboxylic acid (3, 0.12 g, 0.17 mmol) and methylamine (0.01 g, 0.33 mmol) in Ethanol (3 mL) was added triethylamine (0.07 mL, 0.57 mmol) at room temperature was stirred at room temperature for 16 h. After completion, the reaction mixture was concentrated. The crude residue obtained was purified by Prep-HPLC using acetonitrile in water (+0.1% TFA) as eluents to afford the desired product (2R,4S,5R,6R)-6-((1R,2R)-3-(2-(4-chlorophenyl)acetamido)-1,2-dihydroxypropyl)-4-hydroxy-5-((2-(methylamino)-3,4-dioxocyclobut-1-en-1-yl)amino)-2-((6-(prop-2-yn-1-yloxy)hexyl)oxy)tetrahydro-2H-pyran-2-carboxylic acid (Cpd. 191). Yield: 0.055 g, 46%; LCMS m/z 666.45 [M+H]+; ¹H-NMR (400 MHz, Methanol-d₄): δ 8.09 (brs, 1H), 7.31-7.26 (m, 4H), 4.12 (d, J=2.4 Hz, 2H), 3.91-3.86 (m, 2H), 3.78-3.70 (m, 2H), 3.65-3.59 (m, 1H), 3.52-3.45 (m, 6H), 3.41-3.37 (m, 1H), 3.28-3.23 (m, 3H), 2.81 (t, J=2.4 Hz, 1H), 2.70 (dd, J=7.2 Hz and 4.4 Hz, 1H), 1.78 (t, J=12.0 Hz, 1H), 1.57-1.54 (m, 4H), 1.38-1.36 (m, 4H).

### (2R,4S,5R,6R)-5-((2-amino-3,4-dioxocyclobut-1-en-1-yl)amino)-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (Compound 190)

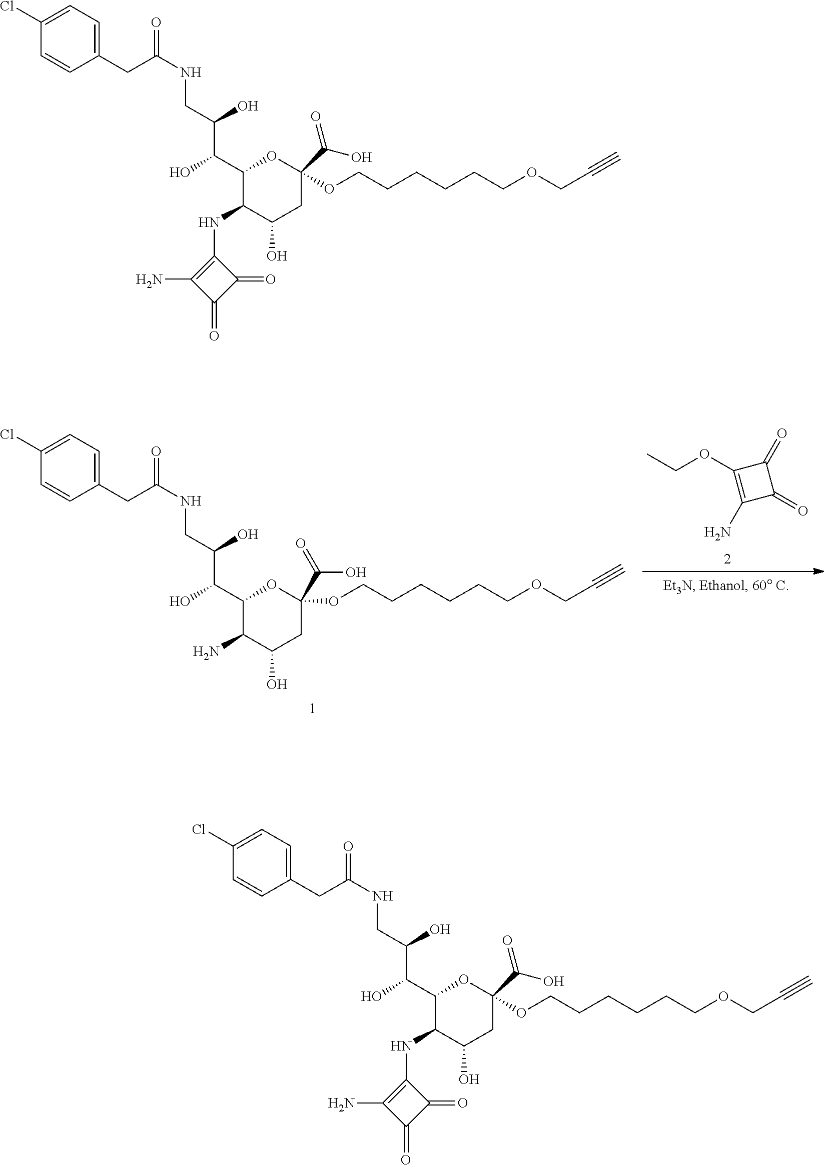

### Synthesis of (2R,4S,5R,6R)-5-((2-amino-3,4-dioxocyclobut-1-en-1-yl)amino)-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (Cpd. 190)

To a stirred solution of (2R,4S,5R,6R)-5-amino-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (0.10 g, 0.18 mmol) in anhydrous dichloromethane (2 mL) was added 3-amino-4-ethoxycyclobut-3-ene-1,2-dione (0.02 g, 0.14 mmol) in ethanol (0.5 mL) and subsequently trimethylamine (0.08 mL, 0.54 mmol) was added to the reaction mixture dropwise at 0° C. The reaction mixture was stirred at 60° C. for 48 h and After completion, the reaction mixture was concentrated. The crude residue obtained was purified by Prep-HPLC using acetonitrile in water (+0.1% TFA) as eluents to afford the desired product (2R,4S,5R,6R)-5-((2-amino-3,4-dioxocyclobut-1-en-1-yl)amino)-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (Cpd. 190). Yield: 0.028 g, 25%; LCMS m/z 652.45 [M+H]+; ¹H-NMR (400 MHz, Methanol-d₄): δ 7.31-7.25 (m, 4H), 4.12 (d, J=2.4 Hz, 2H), 3.92-3.86 (m, 4H), 3.78-3.73 (m, 2H), 3.62 (dd, J=14.0 Hz & 2.8 Hz, 1H), 3.53-3.48 (m, 5H), 3.44-3.39 (m, 1H), 3.31-3.25 (m, 2H), 2.82 (t, J=2.4 Hz, 1H), 2.69 (dd, J=12.8 Hz & 3.6 Hz, 1H), 1.78 (t, J=12.0 Hz, 1H), 1.57-1.55 (m, 4H), 1.40-1.36 (m, 4H).

### (2R,4S,5R,6R)-6-((1R,2R)-3-(2-(4-chlorophenyl)acetamido)-1,2-dihydroxypropyl)-4-hydroxy-5-(2-(methylamino)-2-oxoacetamido)-2-((6-(prop-2-yn-1-yloxy)hexyl)oxy)tetrahydro-2H-pyran-2-carboxylic acid (Compound 189)

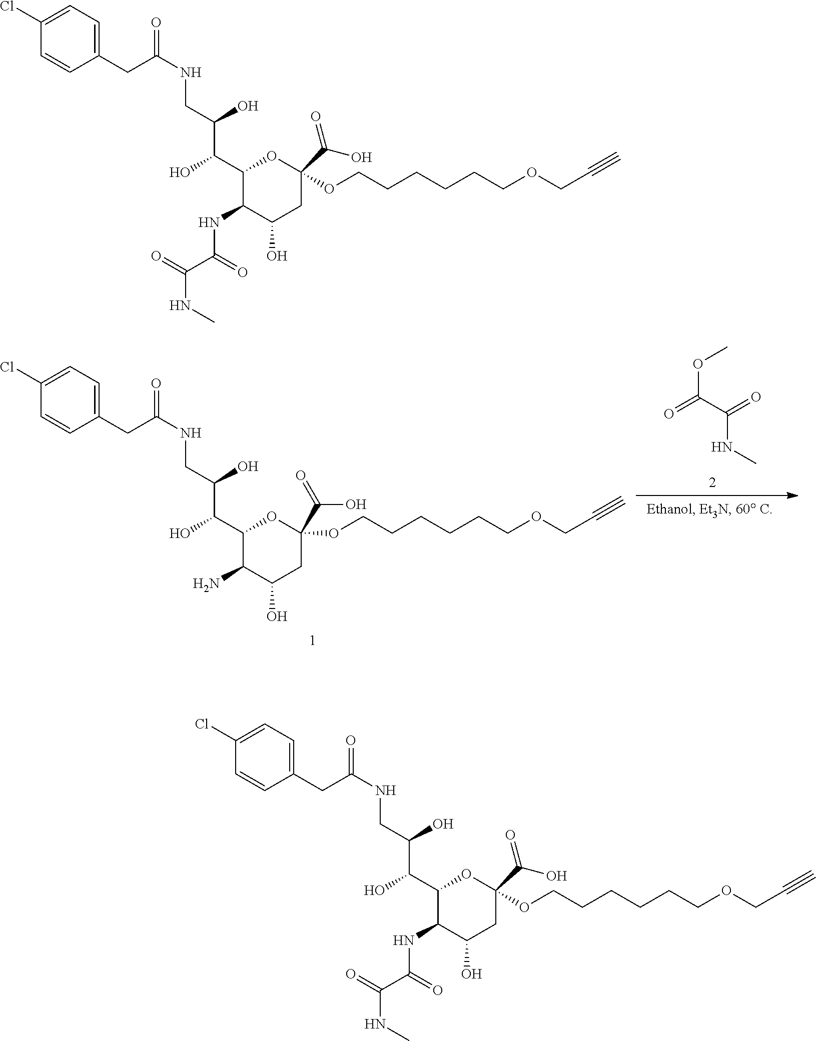

### Synthesis of (2R,4S,5R,6R)-6-((R,2R)-3-(2-(4-chlorophenyl)acetamido)-1,2-dihydroxypropyl)-4-hydroxy-5-(2-(methylamino)-2-oxoacetamido)-2-((6-(prop-2-yn-1-yloxy)hexyl)oxy)tetrahydro-2H-pyran-2-carboxylic acid (Cpd. 189)

To a stirred solution of (2R,4S,5R,6R)-5-amino-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (0.060 g, 0.108 mmol) in anhydrous ethanol (2 mL) was added methyl 2-(methylamino)-2-oxoacetate (0.012 g, 0.108 mmol) in ethanol (0.5 mL) at 0° C. followed by addition of triethylamine (0.08 mL, 0.54 mmol) was stirred at 60° C. for 12 h. After completion, the reaction mixture was concentrated. The crude residue obtained was purified by Prep-HPLC using acetonitrile in water (+0.1% TFA) as eluents to afford the desired product (2R,4S,5R,6R)-6-((1R,2R)-3-(2-(4-chlorophenyl)acetamido)-1,2-dihydroxypropyl)-4-hydroxy-5-(2-(methylamino)-2-oxoacetamido)-2-((6-(prop-2-yn-1-yloxy)hexyl)oxy)tetrahydro-2H-pyran-2-carboxylic acid (Cpd. 189). Yield: 0.019 g, 27%; LCMS m/z 642.40 [M+H]+; ¹HNMR (400 MHz, Methanol-d4): δ 7.31-7.25 (m, 4H), 4.12 (d, J=2.4 Hz, 2H), 3.88-3.81 (m, 4H), 3.78-3.73 (m, 1H), 3.62 (dd, J=13.6 Hz & 2.8 Hz, 1H), 3.53-3.48 (m, 4H), 3.42-3.35 (m, 2H), 3.32-3.26 (m, 1H), 2.83-2.80 (m, 4H), 2.70 (dd, J=12.6 Hz & 4.4 Hz, 1H), 1.72 (t, J=12.0 Hz, 1H), 1.57-1.53 (m, 4H), 1.40-1.35 (m, 4H).

### (2R,4S,5R,6R)-6-((1R,2R)-3-(2-(4-chlorophenyl)acetamido)-1,2-dihydroxypropyl)-5-(3,3-dimethylureido)-4-hydroxy-2-((6-(prop-2-yn-1-yloxy)hexyl)oxy)tetrahydro-2H-pyran-2-carboxylic acid (Compound 85)

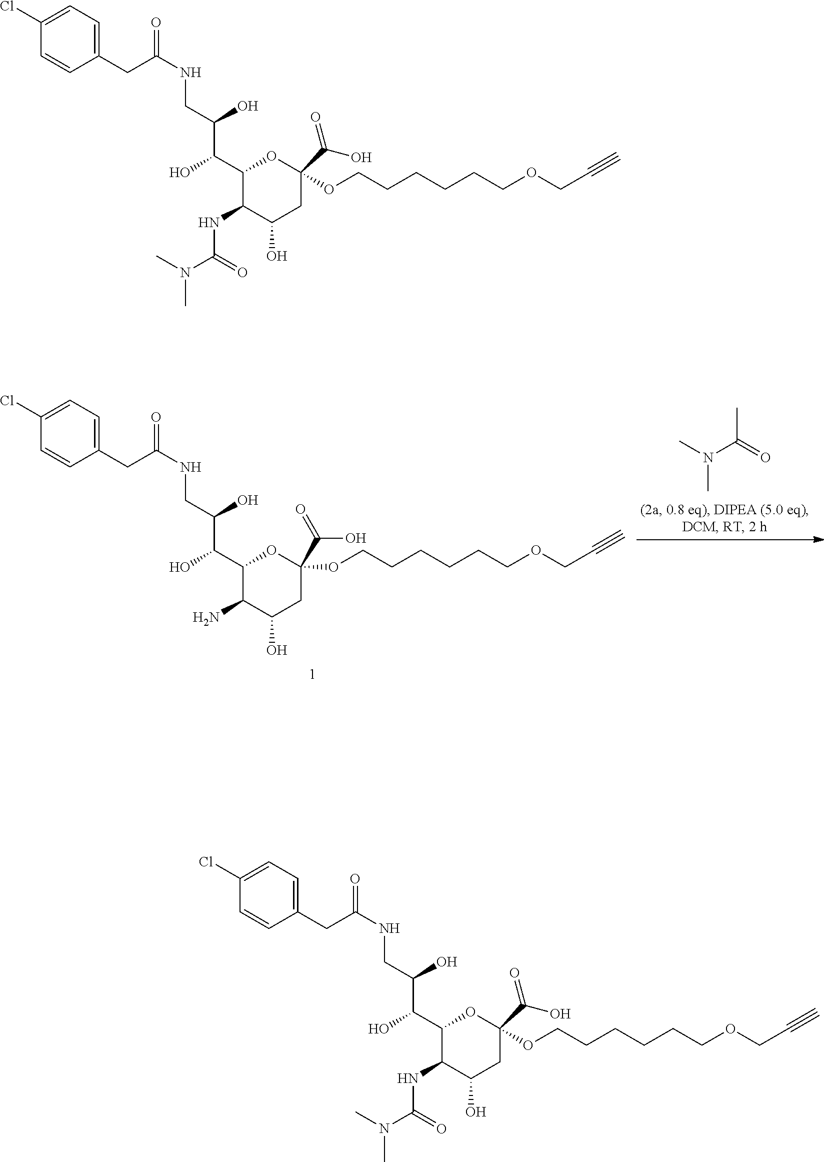

### Synthesis of (2R,4S,5R,6R)-6-((1R,2R)-3-(2-(4-chlorophenyl)acetamido)-1,2-dihydroxypropyl)-5-(3,3-dimethylureido)-4-hydroxy-2-((6-(prop-2-yn-1-yloxy)hexyl)oxy)tetrahydro-2H-pyran-2-carboxylic acid (Cpd. 85)

To a stirred solution of (2R,4S,5R,6R)-5-amino-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (0.050 g, 0.089 mmol) in dichloromethane (3 mL), N-ethylbis(isopropyl)amine (0.045 mL, 0.269 mmol) was added. After that dimethylcarbamic chloride (0.011 g, 0.108 mmol) was added to the reaction mixture at 0° C. then stirred at room temperature for 2 h. After completion, the reaction mixture was concentrated. The crude residue obtained was purified by Prep-HPLC using acetonitrile in water (+0.1% TFA) as eluents to afford the desired product (2R,4S,5R,6R)-6-((1R,2R)-3-(2-(4-chlorophenyl)acetamido)-1,2-dihydroxypropyl)-5-(3,3-dimethylureido)-4-hydroxy-2-((6-(prop-2-yn-1-yloxy)hexyl)oxy)tetrahydro-2H-pyran-2-carboxylic acid (Cpd. 85). Yield: 0.020 g, 35%; LCMS m/z 628.45 [M+H]+; ¹H-NMR (400 MHz, Methanol-d4): δ 8.03-8.00 (m, 0.13 H), 7.35-7.25 (m, 4H), 4.11 (d, J=2.4 Hz, 2H), 3.91-3.86 (m, 1H), 3.80-3.71 (m, 2H), 3.67-3.60 (m, 2H), 3.58-3.50 (m, 5H), 3.48-3.37 (m, 2H), 3.30-3.24 (m, 1H), 2.90 (s, 6H), 2.81 (t, J=2.4 Hz, 1H), 2.72 (dd, J=12.8 Hz, 4.8 Hz, 1H), 1.71 (t, J=12.4 Hz, 1H), 1.57-1.53 (m, 4H), 1.40-1.35 (m, 4H).

### (2R,4S,5R,6R)-6-((1R,2R)-3-(2-(4-chlorophenyl)acetamido)-1,2-dihydroxypropyl)-4-hydroxy-5-(3-methylthioureido)-2-((6-(prop-2-yn-1-yloxy)hexyl)oxy)tetrahydro-2H-pyran-2-carboxylic acid (Compound 84)

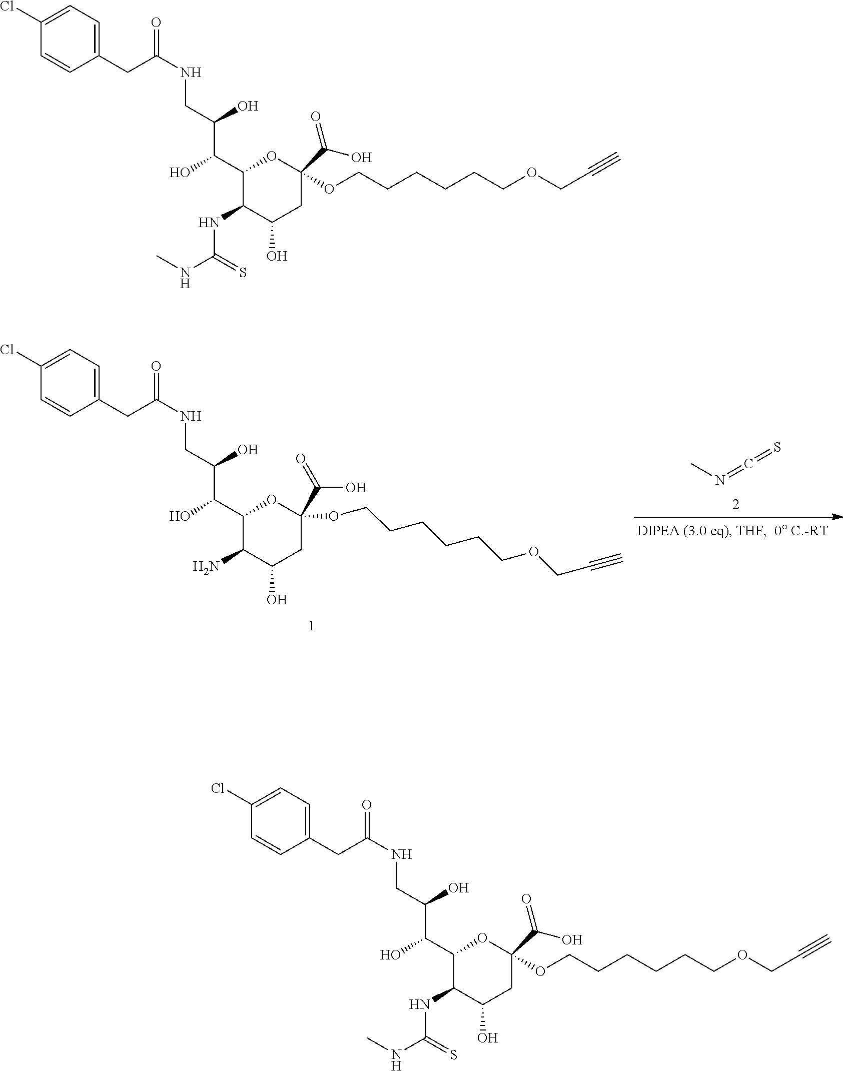

### Synthesis of (2R,4S,5R,6R)-6-((1R,2R)-3-(2-(4-chlorophenyl)acetamido)-1,2-dihydroxypropyl)-4-hydroxy-5-(3-methylthioureido)-2-((6-(prop-2-yn-1-yloxy)hexyl)oxy)tetrahydro-2H-pyran-2-carboxylic acid (Cpd. 84)

To a stirred solution of (2R,4S,5R,6R)-5-amino-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (1, 0.100 g, 0.180 mmol) in anhydrous tetrahydrofuran (3 mL) was added isothiocyanatomethane (2, 0.026 g, 0.359 mmol) in tetrahydrofuran (0.1 mL) at 0° C. followed by addition of N-ethylbis(isopropyl)amine (0.070 mL, 0.539 mmol) was stirred at room temperature for 4 h. After completion, the reaction mixture was concentrated. The crude residue obtained was purified by Prep-HPLC using acetonitrile in water (+0.1% TFA) as eluents to afford the desired product (2R,4S,5R,6R)-6-((1R,2R)-3-(2-(4-chlorophenyl)acetamido)-1,2-dihydroxypropyl)-4-hydroxy-5-(3-methylthioureido)-2-((6-(prop-2-yn-1-yloxy)hexyl)oxy)tetrahydro-2H-pyran-2-carboxylic acid (Cpd. 84). Yield: 0.022 g, 19.4%; LCMS m/z 6320.3 [M+H]+; ¹H-NMR (400 MHz, Methanol-d4): δ 7.38-7.29 (m, 4H), 4.14 (d, J=2.4 Hz, 2H), 3.93-3.89 (m, 1H), 3.80-3.63 (m, 4H), 3.55-3.50 (m, 4H), 3.47-3.41 (m, 2H), 3.36-3.32 (m, 1H), 2.99(bs, 3H), 2.83 (t, J=2.4 Hz, 1H), 2.75-2.71 (dd, J=4.4 Hz, 12.4 Hz, 1H), 1.74 (t, J=12.4 Hz, 1H), 1.59-1.56 (m, 4H), 1.41-1.38 (m, 4H).

### (2R,4S,5R,6R)-5-((2-amino-2-oxoethyl)amino)-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (Compound 182)

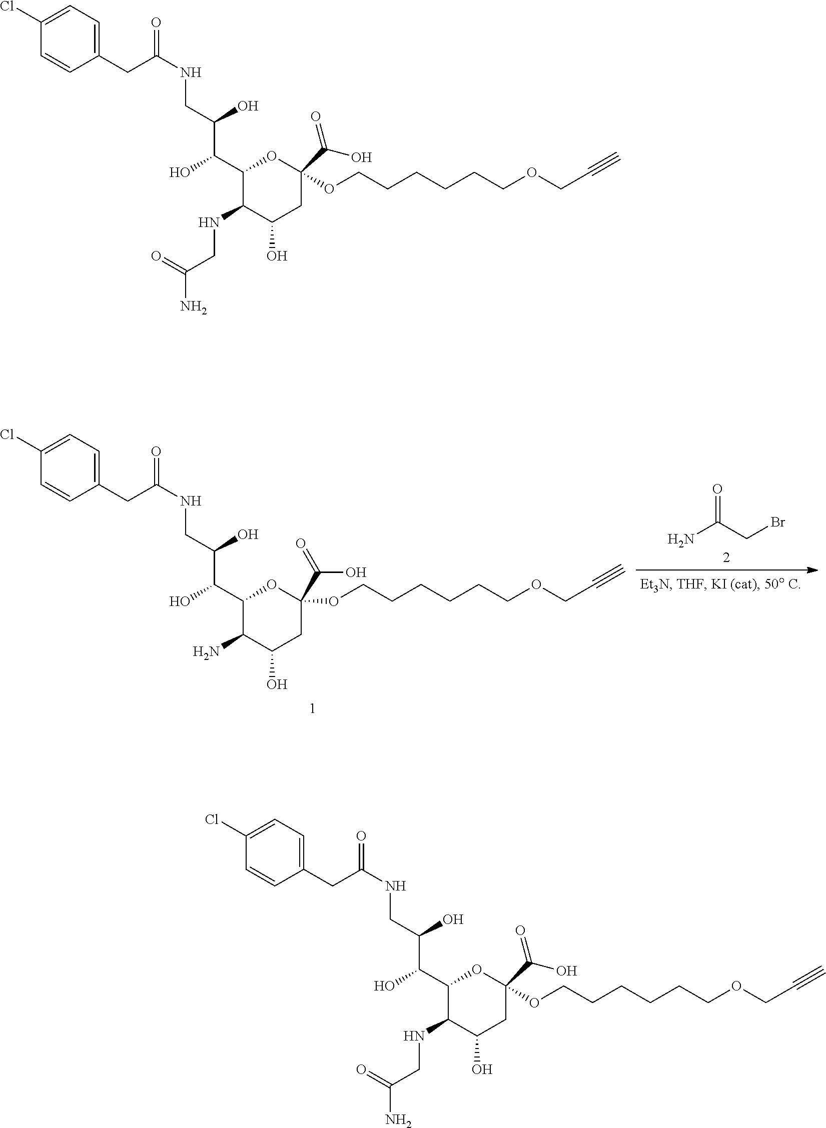 [SMILES: C#CCOCCCCCCO[C@]1(C(=O)O)C[C@H](O)[C@@H](NCC(N)=O)[C@H]([C@H](O)[C@H](O)CNC(=O)Cc2ccc(Cl)cc2)O1 ; C#CCOCCCCCCO[C@]1(C(=O)O)C[C@H](O)[C@@H](N)[C@H]([C@H](O)[C@H](O)CNC(=O)Cc2ccc(Cl)cc2)O1 ; NC(=O)CBr ; C#CCOCCCCCCO[C@]1(C(=O)O)C[C@H](O)[C@@H](NCC(N)=O)[C@H]([C@H](O)[C@H](O)CNC(=O)Cc2ccc(Cl)cc2)O1]

### Synthesis of (2R,4S,5R,6R)-5-((2-amino-2-oxoethyl)amino)-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (Cpd. 182)

To a stirred solution of (2R,4S,5R,6R)-5-amino-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (1, 0.10 g, 0.18 mmol) in tetrahydrofuran (3.0 mL) were added triethylamine (0.07 mL, 0.539 mmol), potassium iodide (cat) and 2-bromoacetamide (2, 0.037 g, 0.27 mmol) subsequently was heated at 50° C. for 1 h. After completion, the reaction mixture was concentrated. The crude residue obtained was purified by Prep-HPLC using acetonitrile in water (+0.1% TFA) as eluents to afford the desired product (2R,4S,5R,6R)-5-((2-amino-2-oxoethyl)amino)-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (Cpd. 182). Yield: 0.028 g, 25%; LCMS m/z 614.30 [M+H]+; ¹HNMR (400 MHz, Methanol-d4): δ 7.32-7.27 (m, 4H), 4.12 (d, J=2.0 Hz, 2H), 3.99-3.92 (m, 4H), 3.86-3.79 (m, 1H), 3.73-3.65 (m, 1H), 3.64-3.58 (m, 2H), 3.55 (brs, 2H), 3.48 (t, J=6.4 Hz, 1H), 3.44-3.38 (m, 2H), 3.03 (t, J=10.0 Hz, 1H), 2.81-2.77 (m, 2H), 1.63-1.49 (m, 5H), 1.34-1.32 (m, 4H).

### (2R,4S,5R,6R)-6-((1R,2R)-3-(2-(4-chlorophenyl)acetamido)-1,2-dihydroxypropyl)-4-hydroxy-5-((2-(methylamino)-2-oxoethyl)amino)-2-((6-(prop-2-yn-1-yloxy)hexyl)oxy)tetrahydro-2H-pyran-2-carboxylic acid (Compound 181)

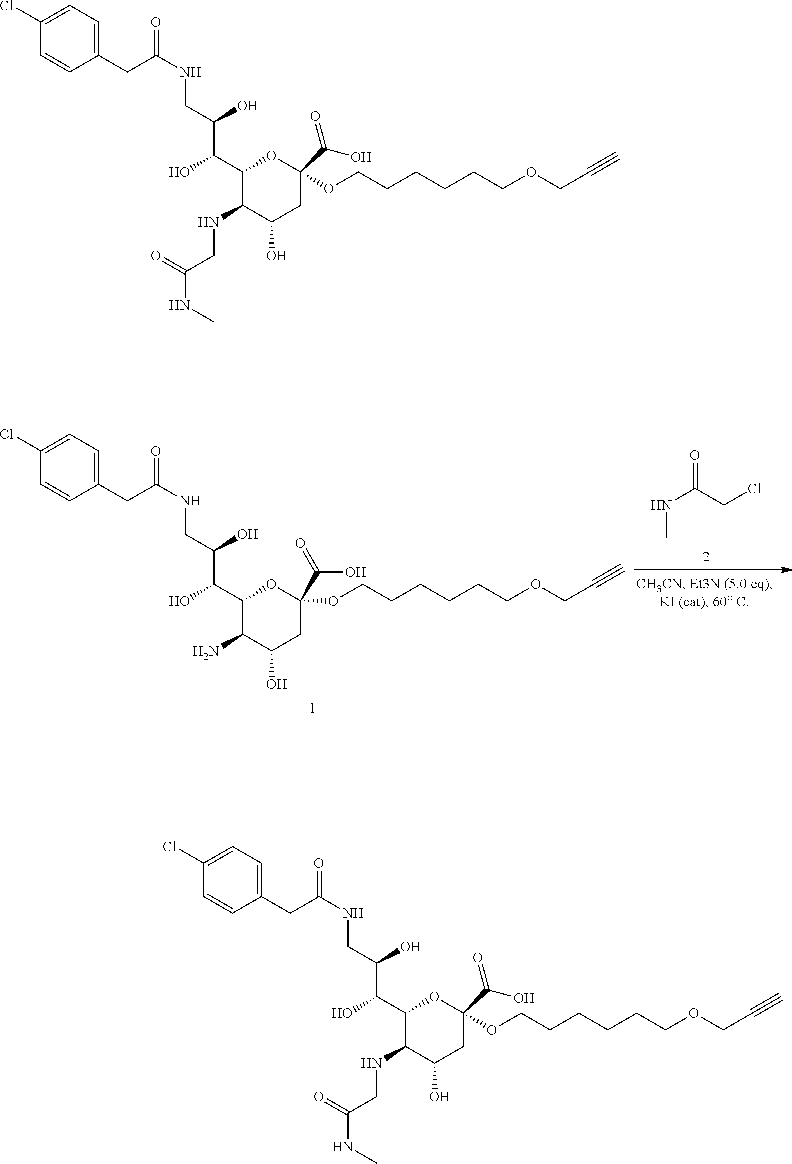

### Synthesis of (2R,4S,5R,6R)-6-((1R,2R)-3-(2-(4-chlorophenyl)acetamido)-1,2-dihydroxypropyl)-4-hydroxy-5-((2-(methylamino)-2-oxoethyl)amino)-2-((6-(prop-2-yn-1-yloxy)hexyl)oxy)tetrahydro-2H-pyran-2-carboxylic acid (Cpd. 181)

To a stirred solution of (2R,4S,5R,6R)-5-amino-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (1, 0.10 g, 0.18 mmol) in acetonitrile (3.0 mL) were added triethylamine (0.07 mL, 0.539 mmol), potassium iodide (cat) and 2-chloro-N-methylacetamide (2, 0.029 g, 0.27 mmol) subsequently was heated at 60° C. for 1 h. After completion, the reaction mixture was concentrated. The crude residue obtained was purified by Prep-HPLC using acetonitrile in water (+0.1% TFA) as eluents to afford the desired product (2R,4S,5R,6R)-6-((1R,2R)-3-(2-(4-chlorophenyl)acetamido)-1,2-dihydroxypropyl)-4-hydroxy-5-((2-(methylamino)-2-oxoethyl)amino)-2-((6-(prop-2-yn-1-yloxy)hexyl)oxy)tetrahydro-2H-pyran-2-carboxylic acid (Cpd. 181). Yield: 0.011 g, 10%; LCMS m/z 628.35 [M+H]+; ¹H-NMR (400 MHz, Methanol-d4): δ 7.32-7.27 (m, 4H), 4.11 (d, J=2.4 Hz, 2H), 3.97-3.94 (m, 2H), 3.88 (brs, 2H), 3.83-3.77 (m, 1H), 3.73-3.68 (m, 1H), 3.65-3.63 (m, 1H), 3.59-3.55 (m, 3H), 3.48 (t, J=3.2 Hz, 2H), 3.46-3.36 (m, 2H), 3.06 (t, J=9.2 Hz, 1H), 2.81-2.76 (m, 5H), 1.63 (t, J=12.0 Hz, 1H), 1.56-1.49 (m, 4H), 1.35-1.29 (m, 4H).

### (2R,4S,5R,6R)-6-((1R,2R)-3-(2-(4-chlorophenyl)acetamido)-1,2-dihydroxypropyl)-5-((2-(dimethylamino)-2-oxoethyl)amino)-4-hydroxy-2-((6-(prop-2-yn-1-yloxy)hexyl)oxy)tetrahydro-2H-pyran-2-carboxylic acid (Compound 180)

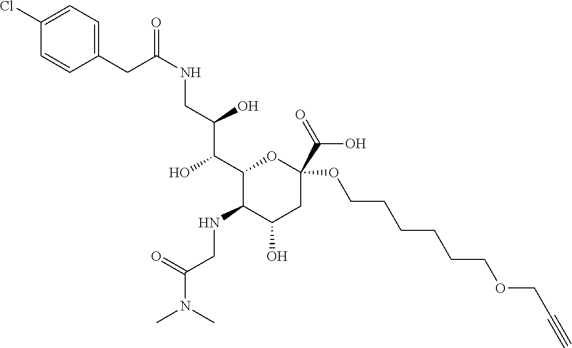 [SMILES: C#CCOCCCCCCO[C@]1(C(=O)O)C[C@H](O)[C@@H](NCC(=O)N(C)C)[C@H]([C@H](O)[C@H](O)CNC(=O)Cc2ccc(Cl)cc2)O1]

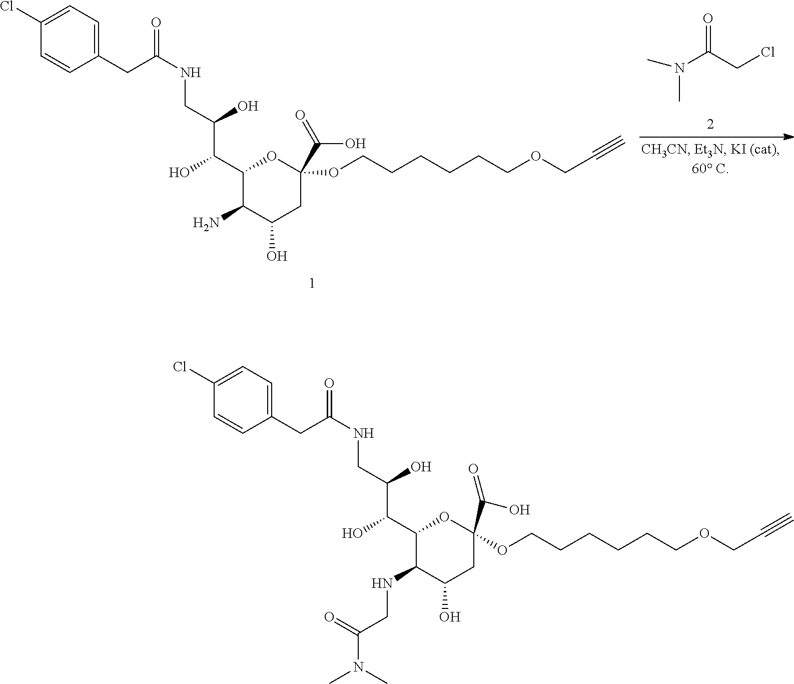

### Synthesis of (2R,4S,5R,6R)-6-((1R,2R)-3-(2-(4-chlorophenyl)acetamido)-1,2-dihydroxypropyl)-5-((2-(dimethylamino)-2-oxoethyl)amino)-4-hydroxy-2-((6-(prop-2-yn-1-yloxy)hexyl)oxy)tetrahydro-2H-pyran-2-carboxylic acid (Cpd. 180)

To a stirred solution of (2R,4S,5R,6R)-5-amino-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (1, 0.10 g, 0.18 mmol) in acetonitrile (3.0 mL) were added triethylamine (0.07 mL, 0.539 mmol), potassium iodide (cat) and 2-chloro-N,N-dimethylacetamide (2, 0.026 g, 0.21 mmol) subsequently was heated at 60° C. for 8 h. After completion, the reaction mixture was concentrated. The crude residue obtained was purified by Prep-HPLC using acetonitrile in water (+0.1% TFA) as eluents to afford the desired product (2R,4S,5R,6R)-6-((1R,2R)-3-(2-(4-chlorophenyl)acetamido)-1,2-dihydroxypropyl)-5-((2-(dimethylamino)-2-oxoethyl)amino)-4-hydroxy-2-((6-(prop-2-yn-1-yloxy)hexyl)oxy)tetrahydro-2H-pyran-2-carboxylic acid (Cpd. 180). Yield: 0.007 g, 8%; LCMS m/z 642.40 [M+H]+; ¹H-NMR (400 MHz, Methanol-d4): δ 7.32-7.27 (m, 4H), 4.26-4.22 (m, 1H), 4.12-4.04 (m, 4H), 3.97-3.87 (m, 2H), 3.73-3.66 (m, 2H), 3.59-3.40 (m, 8H), 2.99 (brs, 3H), 2.93 (brs, 3H), 2.82-2.76 (m, 2H), 1.70 (t, J=12.0 Hz, 2H), 1.55-1.53 (m, 4H), 1.39-1.30 (m, 4H).

### (2R,4S,5R,6R)-6-((1R,2R)-3-(2-(1H-indol-5-yl)acetamido)-1,2-dihydroxypropyl)-4-hydroxy-5-(2-hydroxyacetamido)-2-(oct-7-yn-1-yloxy)tetrahydro-2H-pyran-2-carboxylic acid (Compound 211)

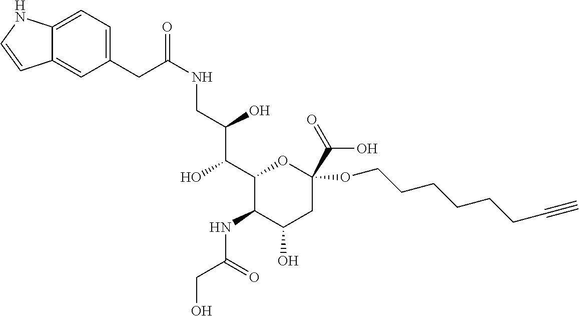 [SMILES: C#CCCCCCCO[C@]1(C(=O)O)C[C@H](O)[C@@H](NC(=O)CO)[C@H]([C@H](O)[C@H](O)CNC(=O)Cc2ccc3[nH]ccc3c2)O1]

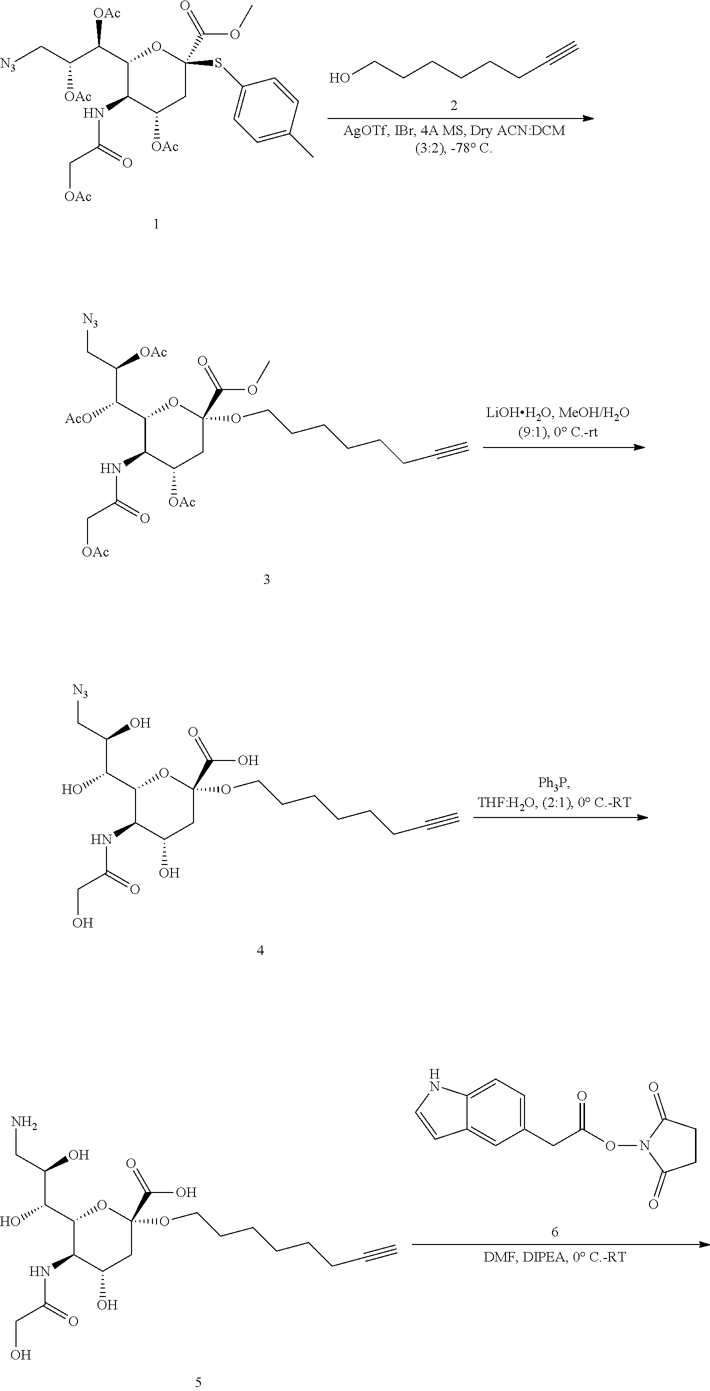

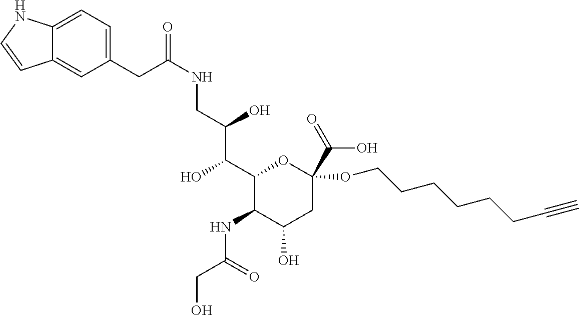 [SMILES: C#CCCCCCCO[C@]1(C(=O)O)C[C@H](O)[C@@H](NC(=O)CO)[C@H]([C@H](O)[C@H](O)CNC(=O)Cc2ccc3[nH]ccc3c2)O1]

### Synthesis of (1R,2R)-1-((2R,3R,4S,6R)-4-acetoxy-3-(2-acetoxyacetamido)-6-(methoxycarbonyl)-6-(oct-7-yn-1-yloxy)tetrahydro-2H-pyran-2-yl)-3-azidopropane-1,2-diyl diacetate (3)

A solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-azidopropane-1,2-diyl diacetate (1, 0.80 g, 1.25 mmol) and ° Ct-7-yn-1-ol (2, 1.58 g, 23.5 mmol), silver(1+) trifluoromethanesulfonate (1.29 g, 5.01 mmol) and activated 4 Å powdered molecular sieves (2.00 g) in anhydrous dichloromethane (55.0 mL) and anhydrous acetonitrile (90.0 mL) was stirred at room temperature for 2 h. The solution was cooled to −78° C. and a solution of iodobromane (0.65 g, 3.13 mmol) in dichloromethane (5.0 mL) was added dropwise. The reaction mixture was stirred at the same temperature for 2 h. After completion, the reaction mixture was quenched by addition of triethylamine (1.0 mL) and warmed up to room temperature. The reaction mixture was filtered, and the filtrate was washed with a saturated solution of sodium bicarbonate and dried, filtered, and concentrated. The crude residue obtained was purified by silica gel column chromatography using ethyl acetate in heptane as eluents to afford the desired product (1R,2R)-1-((2R,3R,4S,6R)-4-acetoxy-3-(2-acetoxyacetamido)-6-(methoxycarbonyl)-6-(oct-7-yn-1-yloxy)tetrahydro-2H-pyran-2-yl)-3-azidopropane-1,2-diyl diacetate (3). Yield: 0.51 g, 63%; ELSD m/z 657.95 [M+H3O]⁺

### Synthesis of (2R,4S,5R,6R)-6-((1R,2R)-3-azido-1,2-dihydroxypropyl)-4-hydroxy-5-(2-hydroxyacetamido)-2-(oct-7-yn-1-yloxy)tetrahydro-2H-pyran-2-carboxylic acid (4)

To a stirred solution of (1R,2R)-1-((2R,3R,4S,6R)-4-acetoxy-3-(2-acetoxyacetamido)-6-(methoxycarbonyl)-6-(oct-7-yn-1-yloxy)tetrahydro-2H-pyran-2-yl)-3-azidopropane-1,2-diyl diacetate (3, 0.60 g, 0.93 mmol) in methanol (20.0 mL) and water (2.0 mL) was added lithium hydroxide monohydrate (0.13 g, 5.62 mmol) at 0° C. The reaction mixture was stirred at room temperature for 6 h. After completion, the reaction mixture was treated with acidic resin (Dowex 50, H⁺) to pH˜6 and the suspension was filtered. The filtrate was concentrated, and the crude residue obtained was purified by trituration with diethyl ether to afford the desired product (2R,4S,5R,6R)-6-((1R,2R)-3-azido-1,2-dihydroxypropyl)-4-hydroxy-5-(2-hydroxyacetamido)-2-(oct-7-yn-1-yloxy)tetrahydro-2H-pyran-2-carboxylic acid (4). Yield: 0.41 g, 95.49%; ELSD m/z 459.05 [M+H]⁺.

### Synthesis of (2R,4S,5R,6R)-6-((1R,2R)-3-amino-1,2-dihydroxypropyl)-4-hydroxy-5-(2-hydroxyacetamido)-2-(oct-7-yn-1-yloxy)tetrahydro-2H-pyran-2-carboxylic acid (5)

To a stirred solution of (2R,4S,5R,6R)-6-((1R,2R)-3-azido-1,2-dihydroxypropyl)-4-hydroxy-5-(2-hydroxyacetamido)-2-(oct-7-yn-1-yloxy)tetrahydro-2H-pyran-2-carboxylic acid (4, 0.60 g, 1.31 mmol) in tetrahydrofuran (18.0 mL) and water (8.0 mL) was added triphenylphosphane (1.72 g, 6.54 mmol) at 0° C. The reaction mixture was allowed to stir at room temperature for 12 h. After completion, the reaction mixture was concentrated. The crude was partitioned between water and ethyl acetate. The aqueous layer was separated and concentrated. The crude residue obtained was purified by prep HPLC using acetonitrile in water (+0.1% TFA) as eluents to afford the desired product (2R,4S,5R,6R)-6-((1R,2R)-3-amino-1,2-dihydroxypropyl)-4-hydroxy-5-(2-hydroxyacetamido)-2-(oct-7-yn-1-yloxy)tetrahydro-2H-pyran-2-carboxylic acid (5). Yield: 0.20 g, 35.34%; ELSD m/z 432.95 [M+H]+; ¹H NMR (400 MHz, Methanol-d₄): δ 4.08-4.04 (m, 3H), 3.88-3.66 (m, 4H), 3.48-3.43 (m, 2H), 3.01-2.96 (m, 1H), 2.78 (dd, J=12.0 Hz & 3.2 Hz, 1H), 2.20-2.16 (m, 3H), 1.73 (t, J=12.0 Hz, 1H), 1.59-1.38 (m, 9H).

### Synthesis of (2R,4S,5R,6R)-6-((1R,2R)-3-(2-(1H-indol-5-yl)acetamido)-1,2-dihydroxypropyl)-4-hydroxy-5-(2-hydroxyacetamido)-2-(oct-7-yn-1-yloxy)tetrahydro-2H-pyran-2-carboxylic acid (Cpd. 211)

To a stirred solution of (2R,4S,5R,6R)-6-((1R,2R)-3-amino-1,2-dihydroxypropyl)-4-hydroxy-5-(2-hydroxyacetamido)-2-(oct-7-yn-1-yloxy)tetrahydro-2H-pyran-2-carboxylic acid (5, 0.06 g, 0.14 mmol) in N,N-dimethylformamide (2.0 mL), N,N-diisopropylethylamine (0.12 mL, 0.69 mmol) and 2,5-dioxopyrrolidin-1-yl 2-(1H-indol-5-yl)acetate (5, 0.03 g, 0.11 mmol) was added at 0° C. then stirred at room temperature for 4 h. After completion, the reaction mixture was concentrated, and the crude residue obtained was purified by prep HPLC using acetonitrile in water (+0.1% FA) to afford the desired product (2R,4S,5R,6R)-6-((1R,2R)-3-(2-(1H-indol-5-yl)acetamido)-1,2-dihydroxypropyl)-4-hydroxy-5-(2-hydroxyacetamido)-2-(oct-7-yn-1-yloxy)tetrahydro-2H-pyran-2-carboxylic acid (Cpd. 211). Yield: 0.027 g, 33%; LCMS (ESI) m/z 590.40 [M+H]+. ¹H NMR (400 MHz, Methanol-d₄): δ 7.48 (s, 1H), 7.33 (d, J=8.4 Hz, 1H), 7.20 (d, J=3.2 Hz, 1H), 7.03 (dd, J=8.4 Hz & 1.6 Hz, 1H), 6.40 (d, J=3.2 Hz, 1H), 3.99 (s, 2H), 3.87-3.63 (m, 6H), 3.60 (s, 2H), 3.41-3.34 (m, 2H), 3.31-3.21 (m, 1H), 2.70 (dd, J=12.0 Hz & 3.2 Hz, 1H), 2.19-2.13 (m, 3H), 1.71 (t, J=12.0 Hz, 1H), 1.55-1.45 (m, 4H), 1.43-1.48 (m, 4H).

### (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-(oct-7-yn-1-yloxy)tetrahydro-2H-pyran-2-carboxylic acid (Compound 212)

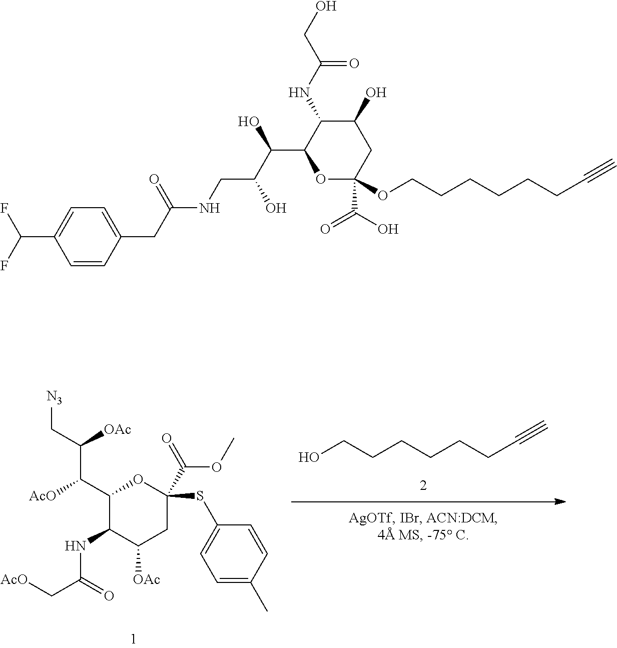

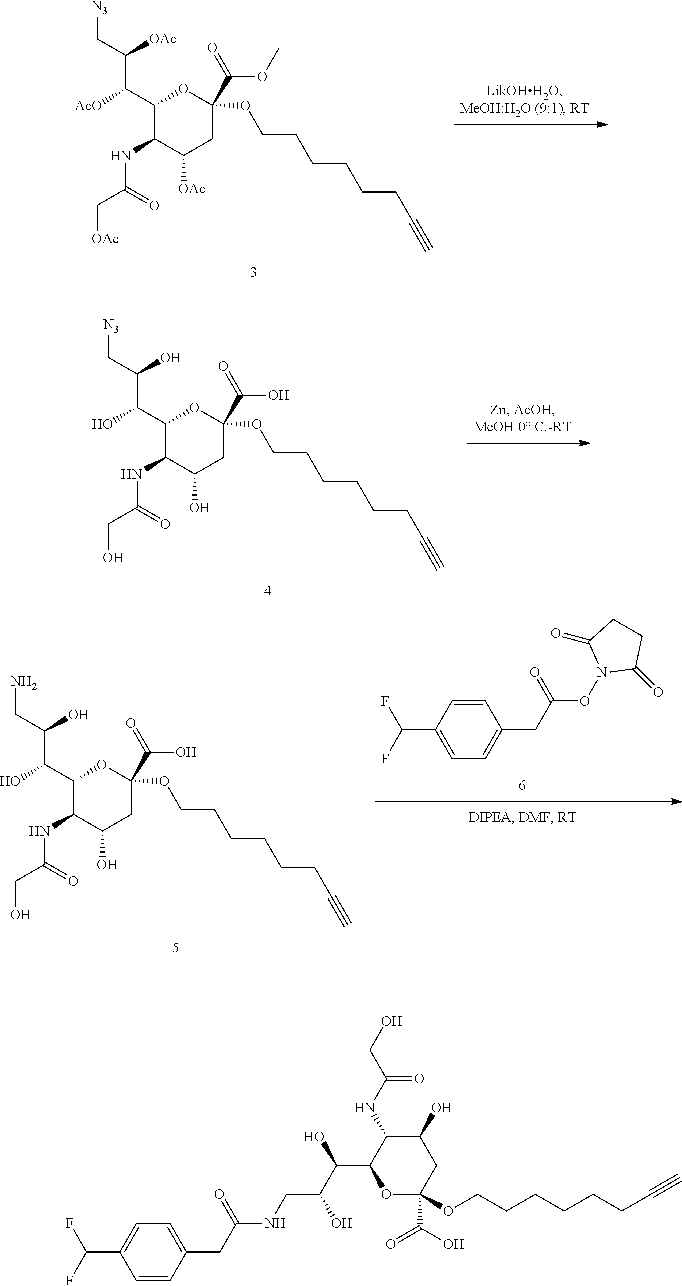 [SMILES: C#CCCCCCCO[C@]1(C(=O)OC)C[C@H](OC(C)=O)[C@@H](NC(=O)COC(C)=O)[C@H]([C@H](OC(C)=O)[C@@H](CN=[N+]=[N-])OC(C)=O)O1 ; C#CCCCCCCO[C@]1(C(=O)O)C[C@H](O)[C@@H](NC(=O)CO)[C@H]([C@H](O)[C@H](O)CN=[N+]=[N-])O1 ; C#CCCCCCCO[C@]1(C(=O)O)C[C@H](O)[C@@H](NC(=O)CO)[C@H]([C@H](O)[C@H](O)CN)O1 ; O=C(Cc1ccc(C(F)F)cc1)ON1C(=O)CCC1=O ; C#CCCCCCCO[C@]1(C(=O)O)C[C@H](O)[C@@H](NC(=O)CO)[C@H]([C@H](O)[C@H](O)CNC(=O)Cc2ccc(C(F)F)cc2)O1]

### Synthesis of (1R,2R)-1-((2R,3R,4S,6R)-4-acetoxy-3-(2-acetoxyacetamido)-6-(methoxycarbonyl)-6-(oct-7-yn-1-yloxy)tetrahydro-2H-pyran-2-yl)-3-azidopropane-1,2-diyl diacetate (3)

A mixture of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-azidopropane-1,2-diyl diacetate (1, 2.00 g, 3.13 mmol) and oct-7-yn-1-ol (2, 1.98 g, 15.66 mmol), silver(1+) trifluoromethanesulfonate (1.61 g, 6.26 mmol) and activated 4 Å powdered molecular sieves (3.00 g) in anhydrous dichloromethane (35.0 mL) and anhydrous acetonitrile (60.0 mL) was stirred at room temperature for 1 h. The solution was cooled to −78° C. and a solution of iodobromane (0.970 g, 4.69 mmol) in dichloromethane (5.0 mL) was added dropwise. The reaction mixture was stirred at the same temperature for 2 h. After completion, the reaction mixture was quenched by addition of triethylamine (3.0 mL) and warmed up to room temperature. The reaction mixture was filtered, and the filtrate was washed with a saturated solution of sodium bicarbonate, dried, filtered, and concentrated. The crude residue obtained was purified by silica gel column chromatography using ethyl acetate in heptane as eluents to afford the desired product (1R,2R)-1-((2R,3R,4S,6R)-4-acetoxy-3-(2-acetoxyacetamido)-6-(methoxycarbonyl)-6-(oct-7-yn-1-yloxy)tetrahydro-2H-pyran-2-yl)-3-azidopropane-1,2-diyl diacetate (3). Yield: 1.70 g, 85%; LCMS m/z 640.85 [M+H]⁺.

### Synthesis of (2R,4S,5R,6R)-6-((1R,2R)-3-azido-1,2-dihydroxypropyl)-4-hydroxy-5-(2-hydroxyacetamido)-2-(oct-7-yn-1-yloxy)tetrahydro-2H-pyran-2-carboxylic acid (4)

To a stirred solution of (1R,2R)-1-((2R,3R,4S,6R)-4-acetoxy-3-(2-acetoxyacetamido)-6-(methoxycarbonyl)-6-(oct-7-yn-1-yloxy)tetrahydro-2H-pyran-2-yl)-3-azidopropane-1,2-diyl diacetate (3, 1.00 g, 1.56 mmol) in methanol (10.0 mL) and water (2.0 mL) was added lithium hydroxide monohydrate (0.335 g, 7.80 mmol) at 0° C. The reaction mixture was stirred at room temperature for 6 h. After completion, the reaction mixture was treated with acidic resin (Dowex 50, H⁺) to pH˜6 and the suspension was filtered. The filtrate was concentrated, and the crude residue obtained was purified by trituration with diethyl ether to afford the desired product (2R,4S,5R,6R)-6-((1R,2R)-3-azido-1,2-dihydroxypropyl)-4-hydroxy-5-(2-hydroxyacetamido)-2-(oct-7-yn-1-yloxy)tetrahydro-2H-pyran-2-carboxylic acid (4) as a crude. Yield: 0.68 g, 95%; LCMS m/z 458.90 [M+H]⁺.

### Synthesis of (2R,4S,5R,6R)-6-((1R,2R)-3-amino-1,2-dihydroxypropyl)-4-hydroxy-5-(2-hydroxyacetamido)-2-(oct-7-yn-1-yloxy)tetrahydro-2H-pyran-2-carboxylic acid (5)

To a stirred solution of (2R,4S,5R,6R)-6-((1R,2R)-3-azido-1,2-dihydroxypropyl)-4-hydroxy-5-(2-hydroxyacetamido)-2-(oct-7-yn-1-yloxy)tetrahydro-2H-pyran-2-carboxylic acid (4, 0.30 g, 0.654 mmol) in methanol (4.0 mL) and water (1.0 mL), were successively added zinc (0.425 g, 6.54 mmol) and acetic acid (0.18 mL, 3.27 mmol) at 0° C., was stirred at room temperature for 2 h. After completion, the reaction mixture was filtered through celite. The filtrate was concentrated, and the crude residue obtained was purified by trituration with diethyl ether to afford the desired product (2R,4S,5R,6R)-6-((1R,2R)-3-amino-1,2-dihydroxypropyl)-4-hydroxy-5-(2-hydroxyacetamido)-2-(oct-7-yn-1-yloxy)tetrahydro-2H-pyran-2-carboxylic acid (5) as a crude. Yield: 0.220 g, 78%; LCMS m/z 433.15 [M+H]⁺.

### Synthesis of (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-(oct-7-yn-1-yloxy)tetrahydro-2H-pyran-2-carboxylic acid (Cpd. 213)

To a stirred solution of (2R,4S,5R,6R)-6-((1R,2R)-3-amino-1,2-dihydroxypropyl)-4-hydroxy-5-(2-hydroxyacetamido)-2-(oct-7-yn-1-yloxy)tetrahydro-2H-pyran-2-carboxylic acid (5, 0.220 g, 0.508 mmol) in N,N-dimethylformamide (3.0 mL) were added N,N-diisopropylethylamine (0.44 mL, 2.54 mmol) and 2,5-dioxopyrrolidin-1-yl 2-(4-(difluoromethyl)phenyl)acetate (6, 0.44 g, 0.508 mmol) at 0° C. then stirred at room temperature for 4 h. After completion, the reaction mixture was concentrated, and the crude residue obtained was purified by prep HPLC using acetonitrile in water (+0.1% TFA) as eluents to afford the desired product (2R,4S,5R,6R)-6-((1R,2R)-3-(2-(4-cyclopropylphenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Cpd. 212). Yield: 0.058 g, 19%; LCMS (ESI) m/z 601.35 [M+H]+. ¹H NMR (400 MHz, methanol-d₄): δ 7.48 (d, J=8.0 Hz, 2H), 7.42 (d, J=8.0 Hz, 2H), 6.73 (t, J=56 Hz, 1H), 4.01 (s, 2H), 3.91-3.86 (m, 1H), 3.85-3.75 (m, 3H), 3.73-3.69 (m, 1H), 3.65 (dd, J=14.0 & 3.2 Hz, 1H), 3.60 (s, 2H), 3.43-3.34 (m, 2H), 3.27-3.22 (m, 1H), 2.72 (dd, J=12.4 Hz & 3.6 Hz, 1H), 2.17-2.14 (m, 3H), 1.72 (t, J=13.6 Hz, 1H), 1.58-1.47 (m, 4H), 1.43-1.37 (m, 4H).

### (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((7-oxo-7-((2-(prop-2-yn-1-yloxy)ethyl)amino)heptyl)oxy)tetrahydro-2H-pyran-2-carboxylic acid (Compound 213)

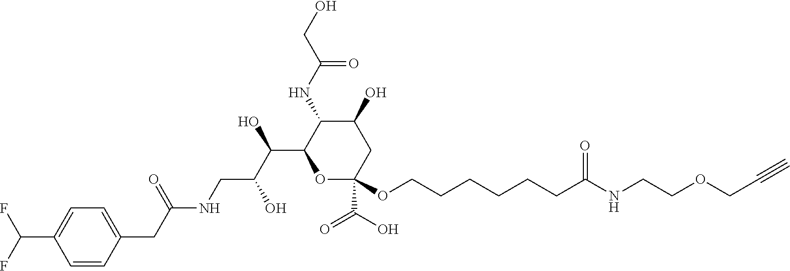 [SMILES: C#CCOCCNC(=O)CCCCCCO[C@]1(C(=O)O)C[C@H](O)[C@@H](NC(=O)CO)[C@H]([C@H](O)[C@H](O)CNC(=O)Cc2ccc(C(F)F)cc2)O1]

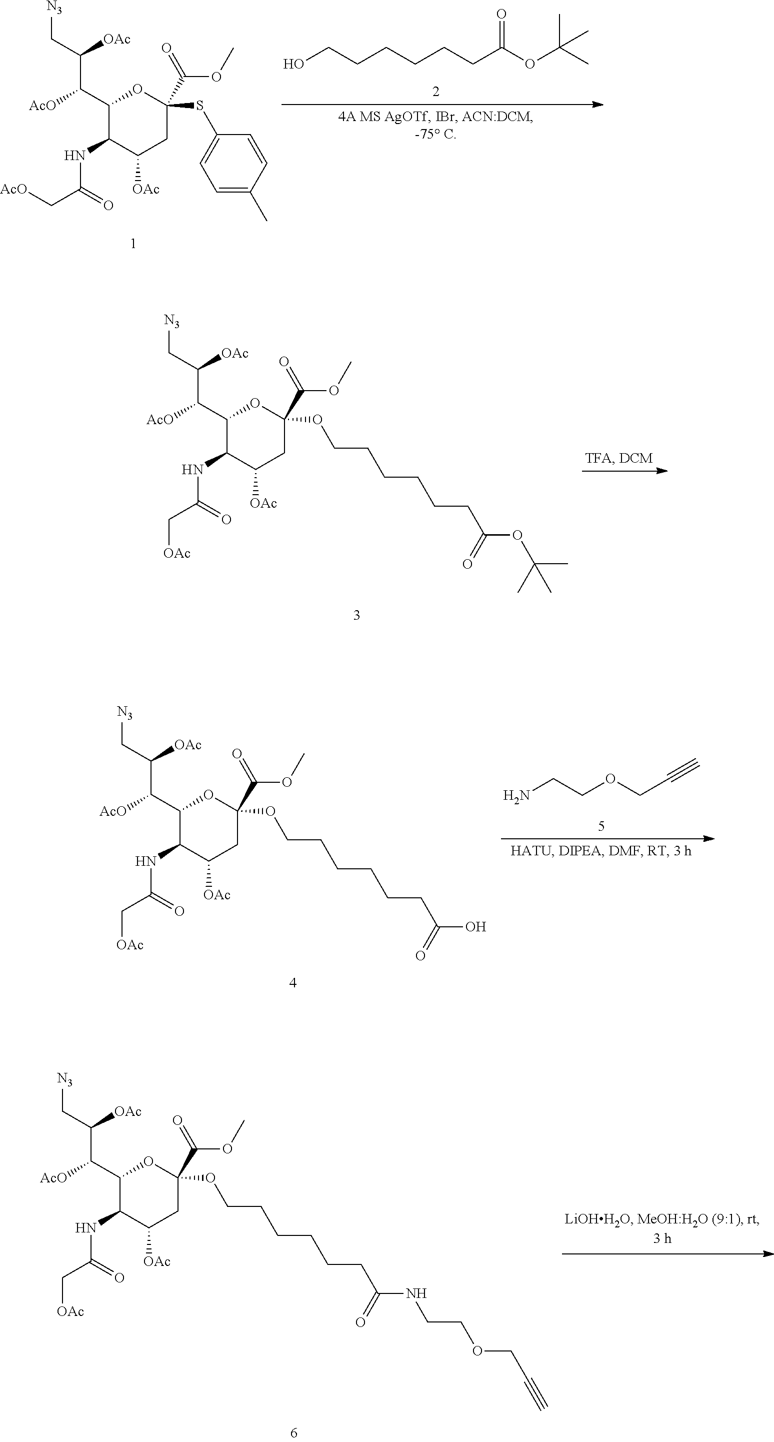

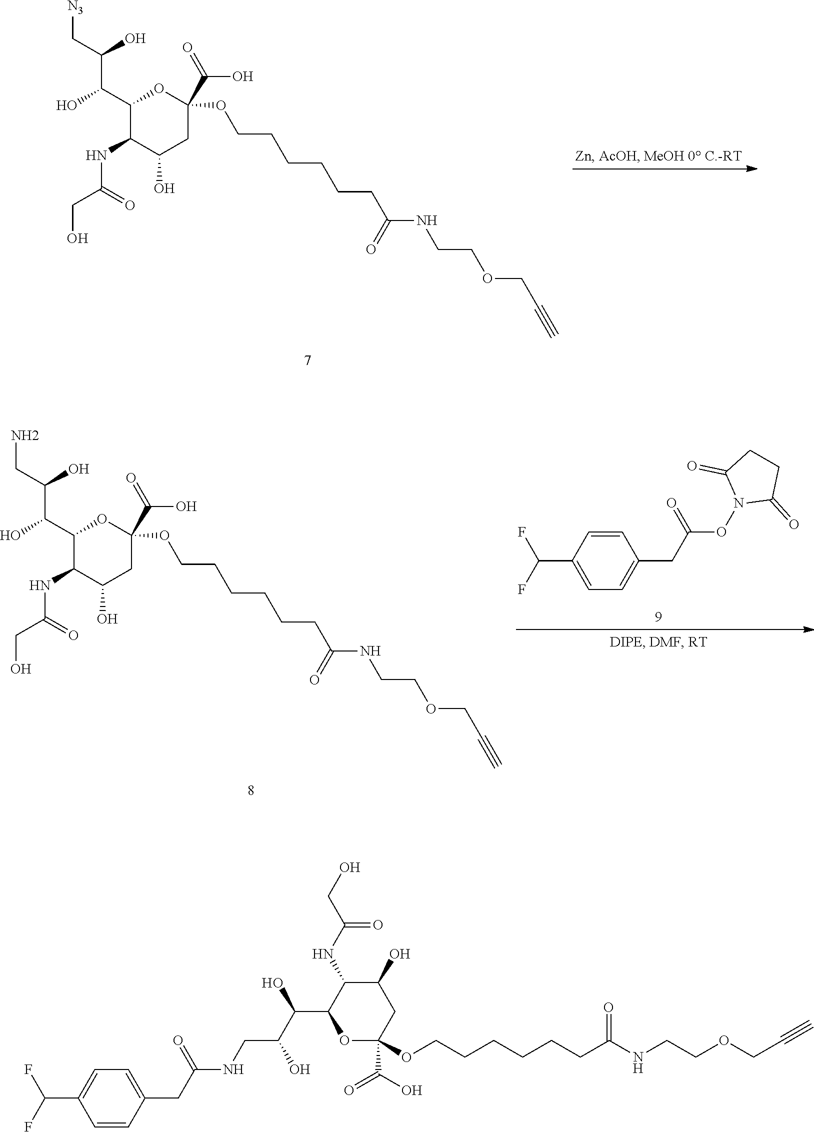 [SMILES: C#CCOCCNC(=O)CCCCCCO[C@]1(C(=O)O)C[C@H](O)[C@@H](NC(=O)CO)[C@H]([C@H](O)[C@H](O)CN=[N+]=[N-])O1 ; C#CCOCCNC(=O)CCCCCCO[C@]1(C(=O)O)C[C@H](O)[C@@H](NC(=O)CO)[C@H]([C@H](O)[C@H](O)CN)O1 ; O=C(Cc1ccc(C(F)F)cc1)ON1C(=O)CCC1=O ; C#CCOCCNC(=O)CCCCCCO[C@]1(C(=O)O)C[C@H](O)[C@@H](NC(=O)CO)[C@H]([C@H](O)[C@H](O)CNC(=O)Cc2ccc(C(F)F)cc2)O1]

### Synthesis of (1R,2R)-1-((2R,3R,4S,6R)-4-acetoxy-3-(2-acetoxyacetamido)-6-((7-(tert-butoxy)-7-oxoheptyl)oxy)-6-(methoxycarbonyl)tetrahydro-2H-pyran-2-yl)-3-azidopropane-1,2-diyl diacetate (3)

A mixture of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-azidopropane-1,2-diyl diacetate (1, 1.00 g, 1.57 mmol) and tert-butyl 7-hydroxyheptanoate (2, 1.58 g, 7.83 mmol), silver(1+) trifluoromethanesulfonate (0.806 g, 3.14 mmol) and activated 4 Å powdered molecular sieves (3.00 g) in anhydrous dichloromethane (15.0 mL) and anhydrous acetonitrile (30.0 mL) was stirred at room temperature for 1 h. The solution was cooled to −78° C. and a solution of iodobromane (0.487 g, 2.35 mmol) in dichloromethane (5.0 mL) was added dropwise. The reaction mixture was stirred at the same temperature for 2 h. After completion, the reaction mixture was quenched by addition of triethylamine (3.0 mL) and warmed up to room temperature. The reaction mixture was filtered, and the filtrate was washed with a saturated solution of sodium bicarbonate and dried, filtered, and concentrated. The crude residue obtained was purified by silica gel column chromatography using ethyl acetate in heptane to (1R,2R)-1-((2R,3R,4S,6R)-4-acetoxy-3-(2-acetoxyacetamido)-6-((7-(tert-butoxy)-7-oxoheptyl)oxy)-6-(methoxycarbonyl)tetrahydro-2H-pyran-2-yl)-3-azidopropane-1,2-diyl diacetate (3). Yield: 0.80 g, 71%; LCMS m/z 714.60 [M+H]+.

### Synthesis of 7-(((2R,4S,5R,6R)-4-acetoxy-5-(2-acetoxyacetamido)-6-((1R,2R)-1,2-diacetoxy-3-azidopropyl)-2-(methoxycarbonyl)tetrahydro-2H-pyran-2-yl)oxy)heptanoic acid (4)

To a stirred solution of (1R,2R)-1-((2R,3R,4S,6R)-4-acetoxy-3-(2-acetoxyacetamido)-6-(methoxycarbonyl)-6-(oct-7-yn-1-yloxy)tetrahydro-2H-pyran-2-yl)-3-azidopropane-1,2-diyl diacetate (3, 0.80 g, 1.12 mmol) in dichloromethane (10.0 mL) was added trifluoroacetic acid (5.0 mL) at 0° C. then stirred at room temperature for 2 h. After completion, the reaction mixture was concentrated. The crude residue obtained was purified by trituration with diethyl ether to afford 7-(((2R,4S,5R,6R)-4-acetoxy-5-(2-acetoxyacetamido)-6-((1R,2R)-1,2-diacetoxy-3-azidopropyl)-2-(methoxycarbonyl)tetrahydro-2H-pyran-2-yl)oxy)heptanoic acid (4). Yield: 0.65 g, 88%; LCMS m/z 660.80 [M+H]⁺.

### Synthesis of (1R,2R)-1-((2R,3R,4S,6R)-4-acetoxy-3-(2-acetoxyacetamido)-6-(methoxycarbonyl)-6-((7-oxo-7-((2-(prop-2-yn-1-yloxy)ethyl)amino)heptyl)oxy)tetrahydro-2H-pyran-2-yl)-3-azidopropane-1,2-diyl diacetate (6)

To a stirred solution of 7-(((2R,4S,5R,6R)-4-acetoxy-5-(2-acetoxyacetamido)-6-((1R,2R)-1,2-diacetoxy-3-azidopropyl)-2-(methoxycarbonyl)tetrahydro-2H-pyran-2-yl)oxy)heptanoic acid (4, 0.60 g, 0.908 mmol) in N,N-dimethylformamide (5.0 mL). 1-[Bis(dimethylamino)methylene]-1H-1,2,3-triazolo[4,5-b]pyridinium 3-oxid hexafluorophosphate (0.690 g, 1.81 mmol), N,N-diisopropylethylamine (0.47 mL, 2.72 mmol) and 2-(prop-2-yn-1-yloxy)ethan-1-amine (5, 0.135 g, 1.36 mmol) was added at 0° C. then stirred at room temperature for 4 h. After completion, the reaction mixture was diluted with water and ethyl acetate. The organic layer was separated and concentrated, and the crude residue obtained was purified by silica gel column chromatography using ethyl acetate in heptane to afford the desired product (1R,2R)-1-((2R,3R,4S,6R)-4-acetoxy-3-(2-acetoxyacetamido)-6-(methoxycarbonyl)-6-((7-oxo-7-((2-(prop-2-yn-1-yloxy)ethyl)amino)heptyl)oxy)tetrahydro-2H-pyran-2-yl)-3-azidopropane-1,2-diyl diacetate (6). Yield: 0.500 g, 74%; LCMS m/z 742.10 [M+H]⁺.

### Synthesis of (2R,4S,5R,6R)-6-((1R,2R)-3-azido-1,2-dihydroxypropyl)-4-hydroxy-5-(2-hydroxyacetamido)-2-((7-oxo-7-((2-(prop-2-yn-1-yloxy)ethyl)amino)heptyl)oxy)tetrahydro-2H-pyran-2-carboxylic acid (7)

To a stirred solution of (1R,2R)-1-((2R,3R,4S,6R)-4-acetoxy-3-(2-acetoxyacetamido)-6-(methoxycarbonyl)-6-((7-oxo-7-((2-(prop-2-yn-1-yloxy)ethyl)amino)heptyl)oxy)tetrahydro-2H-pyran-2-yl)-3-azidopropane-1,2-diyl diacetate (6, 0.50 g, 0.674 mmol) in methanol (5.0 mL) and water (1.0 mL), was added lithium hydroxide monohydrate (0.144 g, 3.37 mmol) at 0° C. The reaction mixture was stirred at room temperature for 6 h. After completion, the reaction mixture was treated with acidic resin (Dowex 50, H⁺) to pH˜6 and the suspension was filtered. The filtrate was concentrated, and the crude residue obtained was purified by trituration with diethyl ether solvent to afford the desired product (2R,4S,5R,6R)-6-((1R,2R)-3-azido-1,2-dihydroxypropyl)-4-hydroxy-5-(2-hydroxyacetamido)-2-((7-oxo-7-((2-(prop-2-yn-1-yloxy)ethyl)amino)heptyl)oxy)tetrahydro-2H-pyran-2-carboxylic acid (7) as a crude. Yield: 0.320 g, 85%; LCMS m/z 559.85 [M+H]⁺.

### Synthesis of (2R,4S,5R,6R)-6-((1R,2R)-3-amino-1,2-dihydroxypropyl)-4-hydroxy-5-(2-hydroxyacetamido)-2-((7-oxo-7-((2-(prop-2-yn-1-yloxy)ethyl)amino)heptyl)oxy)tetrahydro-2H-pyran-2-carboxylic acid (8)

To a stirred solution of (2R,4S,5R,6R)-6-((1R,2R)-3-azido-1,2-dihydroxypropyl)-4-hydroxy-5-(2-hydroxyacetamido)-2-((7-oxo-7-((2-(prop-2-yn-1-yloxy)ethyl)amino)heptyl)oxy)tetrahydro-2H-pyran-2-carboxylic acid (7, 0.320 g, 0.571 mmol) in methanol (4.0 mL) and water (1.0 mL) were successively added zinc (0.373 g, 5.71 mmol) and acetic acid (0.23 mL, 3.75 mmol) at 0° C. then stirred at room temperature for 2 h. After completion, the reaction mixture was filtered through celite. The filtrate was concentrated, and the crude residue obtained was purified by trituration with diethyl ether to afford the desired product (2R,4S,5R,6R)-6-((1R,2R)-3-amino-1,2-dihydroxypropyl)-4-hydroxy-5-(2-hydroxyacetamido)-2-((7-oxo-7-((2-(prop-2-yn-1-yloxy)ethyl)amino)heptyl)oxy)tetrahydro-2H-pyran-2-carboxylic acid (8). Yield: 0.250 g, 82%; LCMS m/z 533.95 [M+H]⁺.

### Synthesis of (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((7-oxo-7-((2-(prop-2-yn-1-yloxy)ethyl)amino)heptyl)oxy)tetrahydro-2H-pyran-2-carboxylic acid (Cpd. 213)

To a stirred solution of (2R,4S,5R,6R)-6-((1R,2R)-3-amino-1,2-dihydroxypropyl)-4-hydroxy-5-(2-hydroxyacetamido)-2-((7-oxo-7-((2-(prop-2-yn-1-yloxy)ethyl)amino)heptyl)oxy)tetrahydro-2H-pyran-2-carboxylic acid (8, 0.250 g, 0.468 mmol) in N,N-dimethylformamide (3.0 mL) were added N,N-diisopropylethylamine (0.40 mL, 2.38 mmol) and 2,5-dioxopyrrolidin-1-yl 2-(4-(difluoromethyl)phenyl)acetate (9, 0.132 g, 0.468 mmol) at 0° C. then stirred at room temperature for 4 h. After completion, the reaction mixture was concentrated, and the crude residue obtained was purified by prep HPLC using in acetonitrile in water (+0.1% TFA) as eluents to afford the desired product (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((7-oxo-7-((2-(prop-2-yn-1-yloxy)ethyl)amino)heptyl)oxy)tetrahydro-2H-pyran-2-carboxylic acid (Cpd. 213). Yield: 0.019 g, 6%; LCMS (ESI) m/z 702.40 [M+H]+. ¹H NMR (400 MHz, methanol-d₄) δ 7.48 (d, J=8.0 Hz, 2H), 7.42 (d, J=8.0 Hz, 2H), 6.73 (t, J=56.4 Hz, 1H), 4.16 (d, J=2.4 Hz, 2H), 4.01 (s, 2H), 3.90-3.86 (m, 1H), 3.83-3.73 (m, 3H), 3.70-3.64 (m, 2H), 3.60 (s, 2H), 3.57 (t, J=5.6 Hz, 2H), 3.43-3.38 (m, 2H), 3.37-3.34 (m, 2H), 3.26-3.21 (m, 1H), 2.84 (t, J=2.0 Hz, 1H), 2.72 (dd, J=12.4 Hz & 3.6 Hz, 1H), 2.17 (t, J=7.6 Hz, 2H), 1.71 (t, J=12.0 Hz, 1H), 1.62-1.52 (m, 4H), 1.34-1.33 (m, 4H).

### (2R,4S,5R,6R)-6-((1R,2R)-3-(2-(4-chloro-3-fluorophenyl)acetamido)-1,2-dihydroxypropyl)-4-hydroxy-5-(2-hydroxyacetamido)-2-((7-oxo-7-((2-(prop-2-yn-1-yloxy)ethyl)amino)heptyl)oxy)tetrahydro-2H-pyran-2-carboxylic acid (Compound 214)

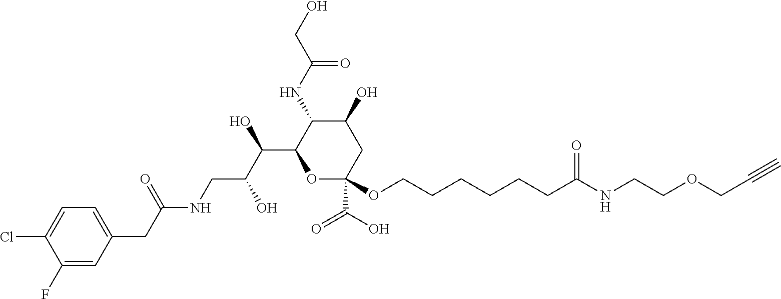 [SMILES: C#CCOCCNC(=O)CCCCCCO[C@]1(C(=O)O)C[C@H](O)[C@@H](NC(=O)CO)[C@H]([C@H](O)[C@H](O)CNC(=O)Cc2ccc(Cl)c(F)c2)O1]

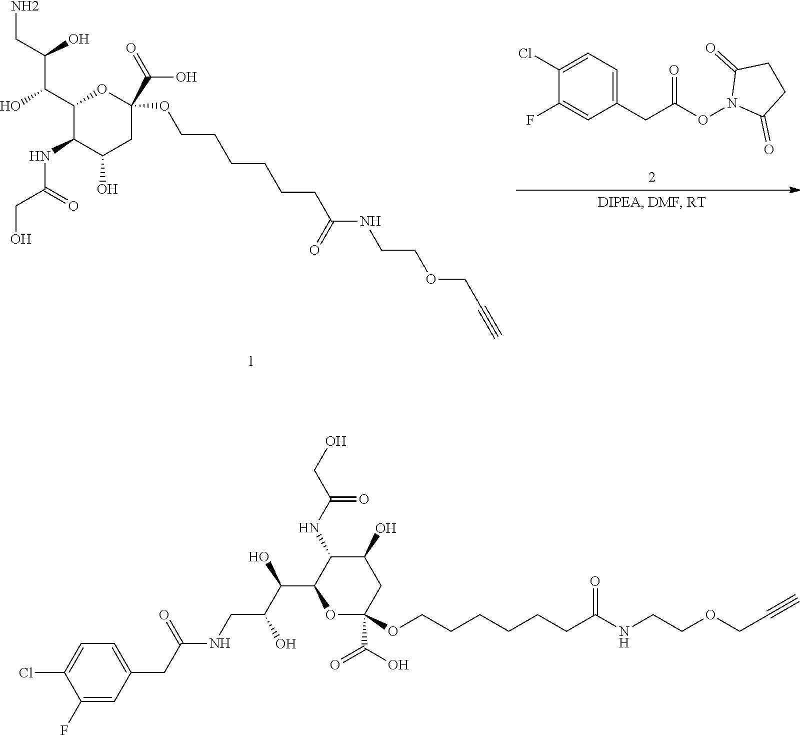 [SMILES: C#CCOCCNC(=O)CCCCCCO[C@]1(C(=O)O)C[C@H](O)[C@@H](NC(=O)CO)[C@H]([C@H](O)[C@H](O)CN)O1 ; O=C(Cc1ccc(Cl)c(F)c1)ON1C(=O)CCC1=O ; C#CCOCCNC(=O)CCCCCCO[C@]1(C(=O)O)C[C@H](O)[C@@H](NC(=O)CO)[C@H]([C@H](O)[C@H](O)CNC(=O)Cc2ccc(Cl)c(F)c2)O1]

### Synthesis of (2R,4S,5R,6R)-6-((1R,2R)-3-(2-(4-chloro-3-fluorophenyl)acetamido)-1,2-dihydroxypropyl)-4-hydroxy-5-(2-hydroxyacetamido)-2-((7-oxo-7-((2-(prop-2-yn-1-yloxy)ethyl)amino)heptyl)oxy)tetrahydro-2H-pyran-2-carboxylic acid (Cpd. 214)

To a stirred solution of (2R,4S,5R,6R)-6-((1R,2R)-3-amino-1,2-dihydroxypropyl)-4-hydroxy-5-(2-hydroxyacetamido)-2-((7-oxo-7-((2-(prop-2-yn-1-yloxy)ethyl)amino)heptyl)oxy)tetrahydro-2H-pyran-2-carboxylic acid (1, 0.200 g, 0.374 mmol) in N,N-dimethylformamide (3.0 mL), N,N-diisopropylethylamine (0.32 mL, 1.87 mmol) and 2,5-dioxopyrrolidin-1-yl 2-(4-chloro-3-fluorophenyl)acetate (2, 0.107 g, 0.374 mmol) was added at 0° C. then stirred at room temperature for 4 h. After completion, the reaction mixture was concentrated, and the crude residue obtained was purified by prep HPLC using acetonitrile in water as eluents (+0.05% TFA) to afford the desired product (2R,4S,5R,6R)-6-((1R,2R)-3-(2-(4-chloro-3-fluorophenyl)acetamido)-1,2-dihydroxypropyl)-4-hydroxy-5-(2-hydroxyacetamido)-2-((7-oxo-7-((2-(prop-2-yn-1-yloxy)ethyl)amino)heptyl)oxy)tetrahydro-2H-pyran-2-carboxylic acid (Cpd. 214). Yield: 0.019 g, 7%; LCMS (ESI) m/z 704.45 [M+H]⁺. ¹H NMR (400 MHz, methanol-d₄): δ 7.39 (t, J=8.4 Hz, 1H), 7.22 (dd, J=10.4 Hz & 1.6 Hz, 1H), 7.11 (dd, J=8.0 Hz & 1.2 Hz, 1H), 4.16 (d, J=2.4 Hz, 2H), 4.01 (s, 2H), 3.90-3.65 (m, 6H), 3.58-3.54 (m, 4H), 3.42-3.33 (m, 4H), 3.30-3.22 (m, 1H), 2.84 (t, J=2.4 Hz, 1H), 2.71 (dd, J=12.4 Hz & 3.6 Hz, 1H), 2.17 (t, J=7.2 Hz, 2H), 1.72 (t, J=12.0 Hz, 1H), 1.63-1.51 (m, 4H), 1.40-1.33 (m, 4H).

### (2R,4S,5R,6R)-6-((1R,2R)-3-(2-([1,1′-biphenyl]-4-yl)acetamido)-1,2-dihydroxypropyl)-2-((2,18-dioxo-6,9,12,15-tetraoxa-3,19-diazapentacosan-25-yl)oxy)-4-hydroxy-5-(2-hydroxyacetamido)tetrahydro-2H-pyran-2-carboxylic acid (Compound 219)

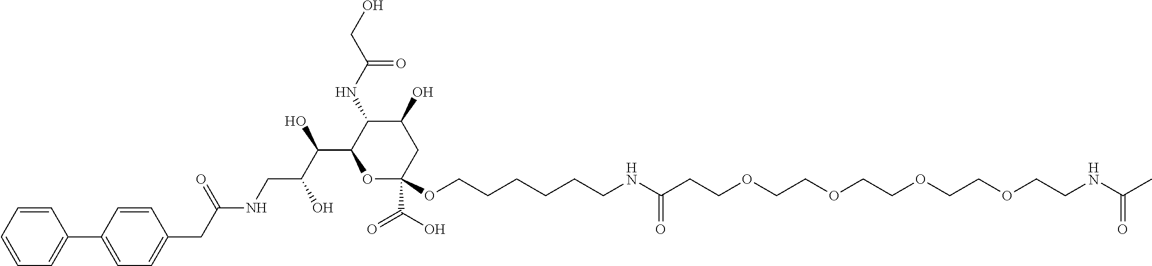 [SMILES: CC(=O)NCCOCCOCCOCCOCCC(=O)NCCCCCCO[C@]1(C(=O)O)C[C@H](O)[C@@H](NC(=O)CO)[C@H]([C@H](O)[C@H](O)CNC(=O)Cc2ccc(-c3ccccc3)cc2)O1]

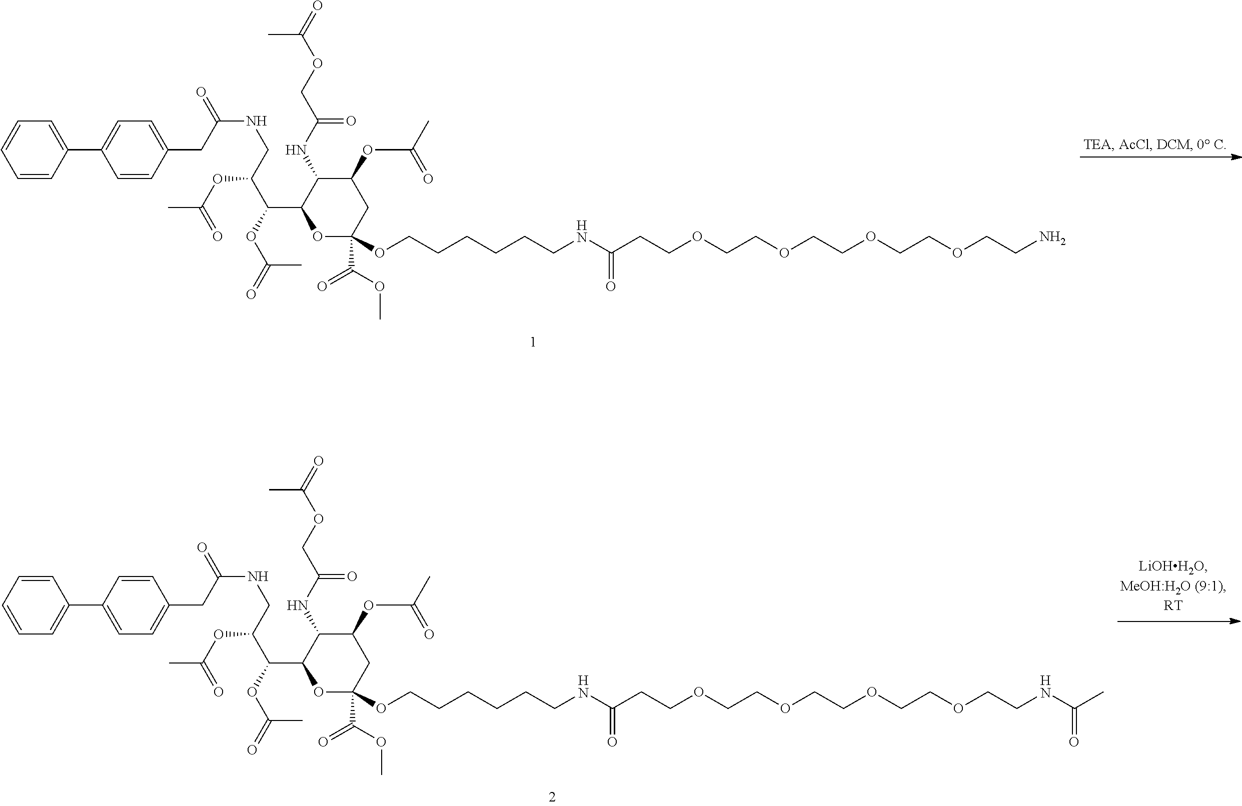 [SMILES: COC(=O)[C@@]1(OCCCCCCNC(=O)CCOCCOCCOCCOCCN)C[C@H](OC(C)=O)[C@@H](NC(=O)COC(C)=O)[C@H]([C@H](OC(C)=O)[C@@H](CNC(=O)Cc2ccc(-c3ccccc3)cc2)OC(C)=O)O1 ; COC(=O)[C@@]1(OCCCCCCNC(=O)CCOCCOCCOCCOCCNC(C)=O)C[C@H](OC(C)=O)[C@@H](NC(=O)COC(C)=O)[C@H]([C@H](OC(C)=O)[C@@H](CNC(=O)Cc2ccc(-c3ccccc3)cc2)OC(C)=O)O1]

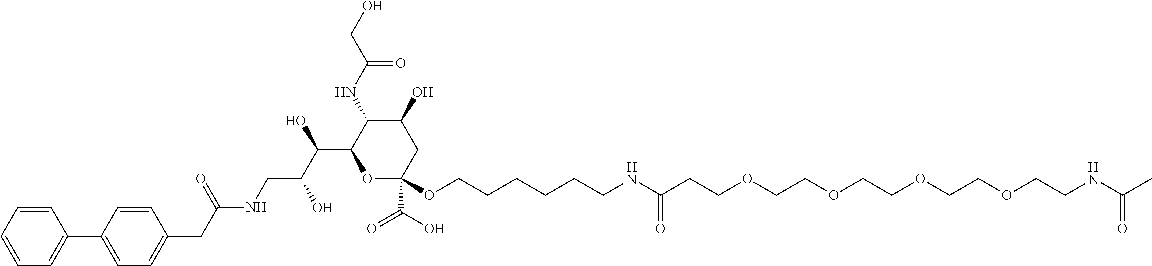 [SMILES: CC(=O)NCCOCCOCCOCCOCCC(=O)NCCCCCCO[C@]1(C(=O)O)C[C@H](O)[C@@H](NC(=O)CO)[C@H]([C@H](O)[C@H](O)CNC(=O)Cc2ccc(-c3ccccc3)cc2)O1]

### Synthesis of (1R,2R)-3-(2-([1,1′-biphenyl]-4-yl)acetamido)-1-((2R,3R,4S,6R)-4-acetoxy-3-(2-acetoxyacetamido)-6-((2,18-dioxo-6,9,12,15-tetraoxa-3,19-diazapentacosan-25-yl)oxy)-6-(methoxycarbonyl)tetrahydro-2H-pyran-2-yl)propane-1,2-diyl diacetate (2)

A stirred solution of (1R,2R)-3-(2-([1,1′-biphenyl]-4-yl)acetamido)-1-((2R,3R,4S,6R)-4-acetoxy-3-(2-acetoxyacetamido)-6-((1-amino-15-oxo-3,6,9,12-tetraoxa-16-azadocosan-22-yl)oxy)-6-(methoxycarbonyl)tetrahydro-2H-pyran-2-yl)propane-1,2-diyl diacetate (1, 0.20 g, 0.190 mmol) in dichloromethane (5.0 mL) was cooled to 0° C. To this solution, triethylamine (0.082 mL, 0.572 mmol) and acetyl chloride (0.013 mL, 0.190 mmol) was added and reaction was maintained at 0° C. for 2 h. After completion, the reaction mixture was diluted with water and dichloromethane. The organic layer was separated and dried, filtered, and concentrated. The crude residue obtained was purified with silica gel column chromatography using ethyl acetate in heptane as eluents to afford the desired product (1R,2R)-3-(2-([1,1′-biphenyl]-4-yl)acetamido)-1-((2R,3R,4S,6R)-4-acetoxy-3-(2-acetoxyacetamido)-6-((6-((3,4-dioxo-2-(prop-2-yn-1-ylamino)cyclobut-1-en-1-yl)amino)hexyl)oxy)-6-(methoxycarbonyl)tetrahydro-2H-pyran-2-yl)propane-1,2-diyl diacetate (2). Yield: 0.130 g, 63%; LCMS (ESI) m/z 1089.05 [M+H]⁺.

### Synthesis of (2R,4S,5R,6R)-6-((1R,2R)-3-(2-([1,1′-biphenyl]-4-yl)acetamido)-1,2-dihydroxypropyl)-2-((2,18-dioxo-6,9,12,15-tetraoxa-3,19-diazapentacosan-25-yl)oxy)-4-hydroxy-5-(2-hydroxyacetamido)tetrahydro-2H-pyran-2-carboxylic acid (Cpd. 219)

To a stirred solution of (1R,2R)-3-(2-([1,1′-biphenyl]-4-yl)acetamido)-1-((2R,3R,4S,6R)-4-acetoxy-3-(2-acetoxyacetamido)-6-((6-((3,4-dioxo-2-(prop-2-yn-1-ylamino)cyclobut-1-en-1-yl)amino)hexyl)oxy)-6-(methoxycarbonyl)tetrahydro-2H-pyran-2-yl)propane-1,2-diyl diacetate (0.130 g, 0.119 mmol) in methanol (3.0 mL) was added lithium hydroxide (0.030 g, 0.714 mmol) solution in water (0.5 mL) at room temperature and the reaction was stirred at room temperature for 6 h. After completion, the mixture was treated with acidic resin (Dowex H form) to reach pH 6 and the reaction was filtered. The filtrate was concentrated. The crude residue obtained was purified with preparative HPLC using acetonitrile in water (+0.1% TFA) as eluents to afford the desired product (2R,4S,5R,6R)-6-((1R,2R)-3-(2-([1,1′-biphenyl]-4-yl)acetamido)-1,2-dihydroxypropyl)-2-((2,18-dioxo-6,9,12,15-tetraoxa-3,19-diazapentacosan-25-yl)oxy)-4-hydroxy-5-(2-hydroxyacetamido)tetrahydro-2H-pyran-2-carboxylic acid (Cpd. 219). Yield: 0.045 g, 42%; LCMS (ESI) m/z 907.55 [M+H]+; 1H NMR (400 MHz, methanol-d4): δ 7.61-7.56 (m, 4H), 7.44-7.37 (m, 4H), 7.33-7.30 (m, 1H), 4.01 (s, 2H), 3.90-3.86 (m, 1H), 3.84-3.73 (m, 3H), 3.72-3.66 (m, 4H), 3.63-3.57 (m, 14H), 3.52 (t, J=5.2 Hz, 2H), 3.39-3.33 (m, 4H), 3.27-3.22 (m, 1H), 3.16-3.12 (m, 2H), 2.70 (dd, J=12.40 & 4.0 Hz, 1H), 2.41 (t, J=6.4 Hz, 1H), 1.93 (s, 3H), 1.72 (t, J=12.4 Hz, 1H), 1.53-1.44 (m, 4H), 1.34-1.31 (m, 4H).

### (2R,4S,5R,6R)-2-((4-(but-3-yn-1-yloxy)benzyl)oxy)-6-((1R,2R)-3-(2-(4-chlorophenyl)acetamido)-1,2-dihydroxypropyl)-4-hydroxy-5-ureidotetrahydro-2H-pyran-2-carboxylic acid (Compound 205)

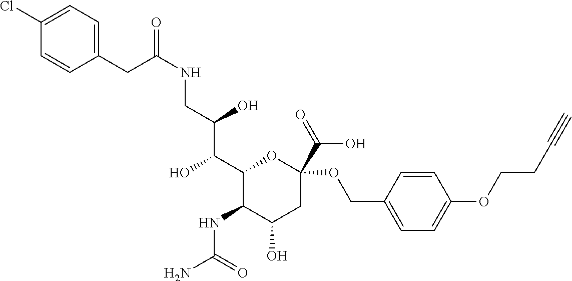 [SMILES: C#CCCOc1ccc(CO[C@]2(C(=O)O)C[C@H](O)[C@@H](NC(N)=O)[C@H]([C@H](O)[C@H](O)CNC(=O)Cc3ccc(Cl)cc3)O2)cc1]

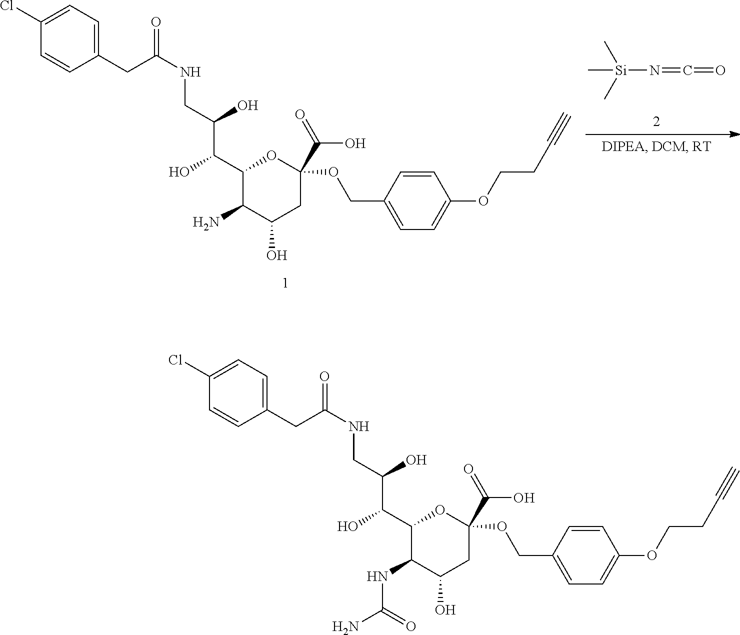

### Synthesis of (2R,4S,5R,6R)-2-((4-(but-3-yn-1-yloxy)benzyl)oxy)-6-((1R,2R)-3-(2-(4-chlorophenyl)acetamido)-1,2-dihydroxypropyl)-4-hydroxy-5-ureidotetrahydro-2H-pyran-2-carboxylic acid (Cpd. 205)

To a stirred solution of (2R,4S,5R,6R)-5-amino-2-((4-(but-3-yn-1-yloxy)benzyl)oxy)-6-((1R,2R)-3-(2-(4-chlorophenyl)acetamido)-1,2-dihydroxypropyl)-4-hydroxytetrahydro-2H-pyran-2-carboxylic acid (1, 0.15 g, 0.26 mmol) in anhydrous dichloromethane (2 mL) was added isocyanatotrimethylsilane (2, 0.044 g, 0.39 mmol) in dichloromethane (0.1 mL) at 0° C. followed by addition of N-ethylbis(isopropyl)amine (0.13 mL, 0.78 mmol) was stirred at room temperature for 2 h. After completion, the reaction mixture was concentrated. The crude residue obtained was purified by Prep-HPLC using acetonitrile in water (+0.1% TFA) as eluents to afford the desired product (2R,4S,5R,6R)-2-((4-(but-3-yn-1-yloxy)benzyl)oxy)-6-((1R,2R)-3-(2-(4-chlorophenyl)acetamido)-1,2-dihydroxypropyl)-4-hydroxy-5-ureidotetrahydro-2H-pyran-2-carboxylic acid (Cpd. 205). Yield: 0.043 g, 26.68%; LCMS m/z 620.35 [M+H]+; ¹H-NMR (400 MHz, Methanol-d₄): δ 7.29-7.24 (m, 4H), 6.89 (d, J=8.4 Hz, 2H), 4.74 (d, J=10.8 Hz, 1H), 4.41 (d, J=10.8 Hz, 1H), 4.08 (t, J=6.8 Hz, 2H), 3.95 (td, J=8.4 Hz & 3.2 Hz, 1H), 3.68 (dd, J=13.6 Hz & 3.2 Hz, 1H), 3.63-3.60 (m, 1H), 3.57 (brs, 2H), 3.54-3.39 (m, 3H), 3.31-3.26 (m, 1H), 2.73 (dd, J=12.6 Hz & 4.4 Hz, 1H), 2.64 (td, J=6.8 Hz & 3.0 Hz, 2H), 2.33 (t, J=2.8 Hz, 1H), 1.78 (t, J=12.0 Hz, 1H).

### (2R,4S,5R,6R)-6-((1R,2R)-3-(2-(4-chlorophenyl)acetamido)-1,2-dihydroxypropyl)-4-hydroxy-5-(2-hydroxyacetamido)-2-((6-(pent-4-ynamido)hexyl)oxy)tetrahydro-2H-pyran-2-carboxylic acid (Compound 217)

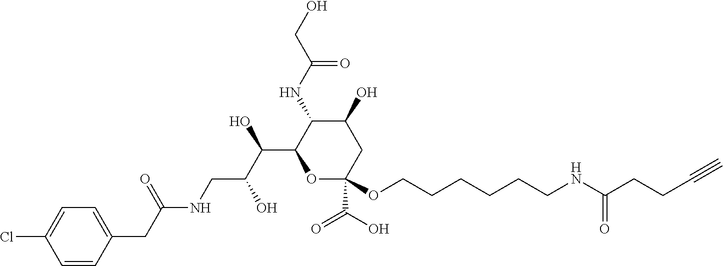 [SMILES: C#CCCC(=O)NCCCCCCO[C@]1(C(=O)O)C[C@H](O)[C@@H](NC(=O)CO)[C@H]([C@H](O)[C@H](O)CNC(=O)Cc2ccc(Cl)cc2)O1]

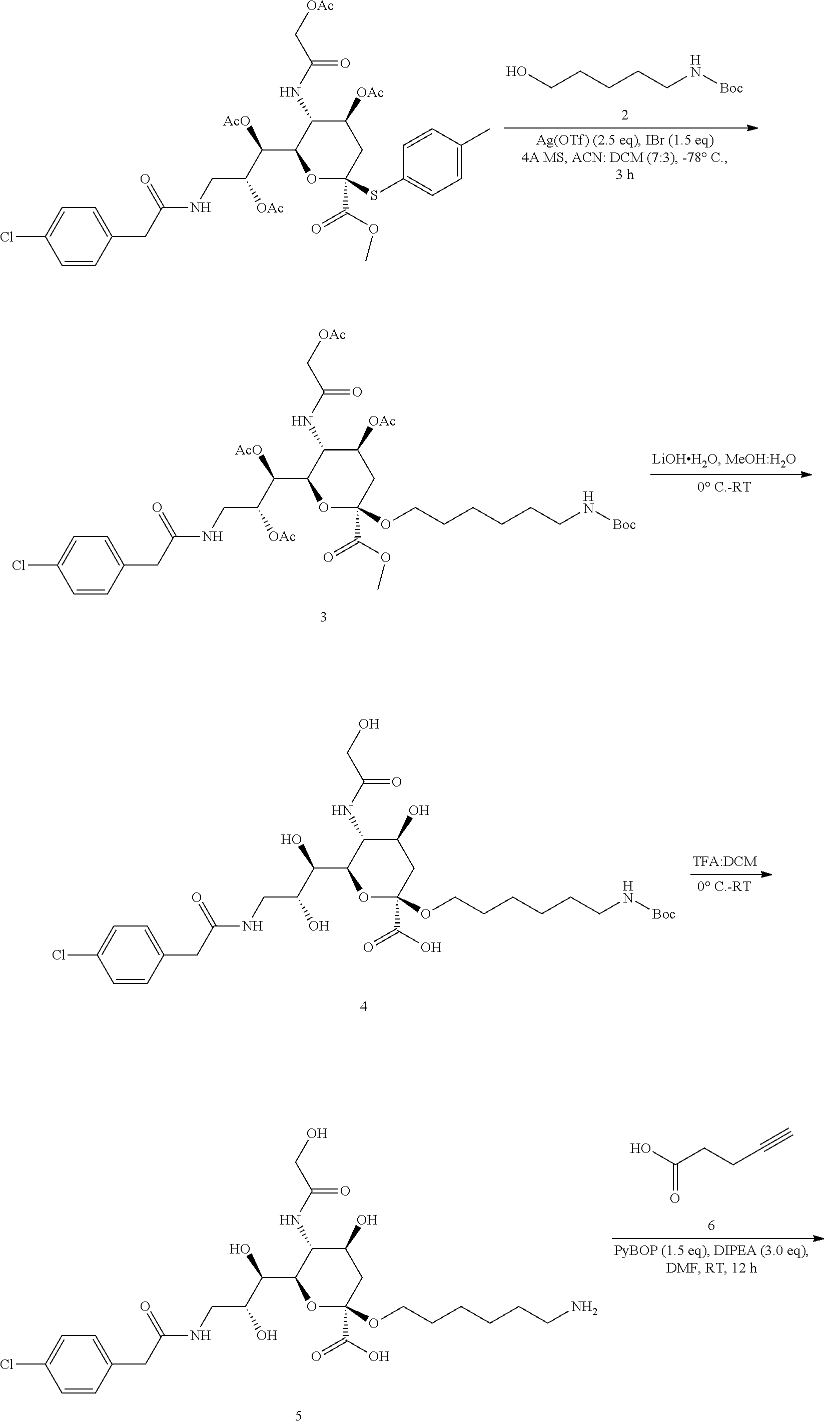

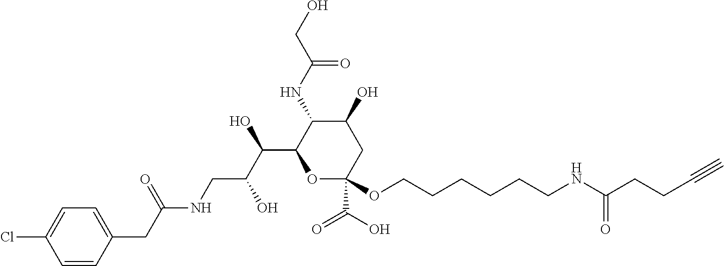 [SMILES: C#CCCC(=O)NCCCCCCO[C@]1(C(=O)O)C[C@H](O)[C@@H](NC(=O)CO)[C@H]([C@H](O)[C@H](O)CNC(=O)Cc2ccc(Cl)cc2)O1]

### Synthesis of (1R,2R)-1-((2R,3R,4S,6R)-4-acetoxy-3-(2-acetoxyacetamido)-6-((6-((tert-butoxycarbonyl)amino)hexyl)oxy)-6-(methoxycarbonyl)tetrahydro-2H-pyran-2-yl)-3-(2-(4-chlorophenyl)acetamido)propane-1,2-diyl diacetate (3)

A mixture of (1R,2R)-1-((2R,3R,4S,6S)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (1, 1.5 g, 1.96 mmol), tert-butyl (5-hydroxypentyl)carbamate (2, 2.13 g, 9.8 mmol), silver trifluoromethanesulfonate (1.01 g, 3.92 mmol) and activated 4 Å powdered molecular sieves (1.5 g) in anhydrous dichloromethane (55.0 mL) and acetonitrile (90.0 mL) was stirred at room temperature for 2 h. A solution of iodine mono bromide (0.60 g, 2.94 mmol) in dichloromethane (5.0 mL) was added at −78° C. and the reaction mixture was stirred at the same temperature for 2 h. After completion, the reaction mixture was quenched by addition of triethyl amine (0.2 mL) and warmed up to room temperature. The reaction mixture was filtered. The filtrate was washed with an aqueous saturated solution of sodium thiosulphate, dried, filtered and concentrated. The crude residue obtained was purified by silica gel column chromatography using ethyl acetate in heptane to afford the desired product (1R,2R)-1-((2R,3R,4S,6R)-4-acetoxy-3-(2-acetoxyacetamido)-6-((6-((tert-butoxycarbonyl)amino)hexyl)oxy)-6-(methoxycarbonyl)tetrahydro-2H-pyran-2-yl)-3-(2-(4-chlorophenyl)acetamido)propane-1,2-diyl diacetate (3). Yield: 1.10 g, 65%; LCMS (ESI) m/z 857.70 [M+H]⁺.

### Synthesis of (2R,4S,5R,6R)-2-((6-((tert-butoxycarbonyl)amino)hexyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (4)

To a stirred solution of (1R,2R)-1-((2R,3R,4S,6R)-4-acetoxy-3-(2-acetoxyacetamido)-6-((6-((tert-butoxycarbonyl)amino)hexyl)oxy)-6-(methoxycarbonyl)tetrahydro-2H-pyran-2-yl)-3-(2-(4-chlorophenyl)acetamido)propane-1,2-diyl diacetate (3, 1.10 g, 1.28 mmol) in methanol (10.0 mL) and water (1.0 mL) was added lithium hydroxide monohydrate (0.153 g, 6.41 mmol) at 0° C. The reaction mixture was stirred at room temperature for 4 h. After completion the reaction mixture was treated with acidic resin (Dowex 50, H⁺) to pH˜6 and the suspension was filtered. The filtrate was concentrated and the crude residue obtained was triturated with diethyl ether then dried under high vacuum to afford the desired product (2R,4S,5R,6R)-2-((6-((tert-butoxycarbonyl)amino)hexyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (4). Yield: 0.80 g, 92%; LCMS m/z 675.80 [M+H]⁺.

### Synthesis of (2R,4S,5R,6R)-2-((6-aminohexyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (5)

To a solution of (2R,4S,5R,6R)-2-((6-((tert-butoxycarbonyl)amino)hexyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (4, 0.80 g, 1.18 mmol), in anhydrous dichloromethane (10.0 mL), trifluoroacetic acid (2.0 mL) was added at 0° C. and the reaction mixture was stirred at room temperature for 4 h. After completion, the solvent was concentrated and dried under high vacuum to afford the desired product (2R,4S,5R,6R)-2-((6-aminohexyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (5) as a crude. The crude residue obtained was directly used for the next step without further purification. Yield: 0.60 g, Crude salt; LCMS (ESI) m/z 576.39 [M+H]⁺.

### Synthesis of (2R,4S,5R,6R)-6-((1R,2R)-3-(2-(4-chlorophenyl)acetamido)-1,2-dihydroxypropyl)-4-hydroxy-5-(2-hydroxyacetamido)-2-((6-(pent-4-ynamido)hexyl)oxy)tetrahydro-2H-pyran-2-carboxylic acid (Cpd. 217)

To a stirred solution of (2R,4S,5R,6R)-2-((6-aminohexyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (5, 0.20 g, 0.347 mmol) and pent-4-ynoic acid (6, 0.041 g, 0.417 mmol) in N,N dimethylformamide (3 mL) were added 1H-1,2,3-benzotriazol-1-yloxy-tri-1-pyrrolidinylphosphonium hexafluoridophosphate(1-) (0.271 g, 0.521 mmol) and ethylbis(propan-2-yl)amine (0.303 mL, 1.74 mmol) sequentially at 0° C. and reaction mixture was stirred at room temperature for 16 h. After completion the reaction mixture was concentrated. The crude residue obtained was purified by prep HPLC using acetonitrile in water (+0.1% TFA) as eluents to afford the desired product (2R,4S,5R,6R)-6-((1R,2R)-3-(2-(4-chlorophenyl)acetamido)-1,2-dihydroxypropyl)-4-hydroxy-5-(2-hydroxyacetamido)-2-((6-(pent-4-ynamido)hexyl)oxy) tetrahydro-2H-pyran-2-carboxylic acid (Cpd. 217). Yield: 0.030 g, 13.7%; LCMS m/z 656.4 [M+H]+; ¹H NMR (400 MHz, Methanol-d₄): δ 7.31-7.24 (m, 4H), 4.01 (m, 2H), 3.88-3.63 (m, 6H), 3.52 (bs, 2H), 3.41-3.31 (m, 2H), 3.25-3.12 (m, 3H), 2.74-2.70 (dd, J=3.6 Hz, 12.0 Hz, 1H), 2.48-2.43 (m, 2H), 2.38-2.34 (m, 2H), 2.25 (t, J=2.8 Hz, 1H), 1.71 (t, J=11.6 Hz, 1H), 1.56-1.46 (m, 4H), 1.36-1.35 (m, 4H).

### (2R,4S,5R,6R)-6-((1R,2R)-3-(2-(4-chlorophenyl)acetamido)-1,2-dihydroxypropyl)-4-hydroxy-5-(2-hydroxyacetamido)-2-((6-(3-(2-(prop-2-yn-1-yloxy)ethyl)ureido)hexyl)oxy)tetrahydro-2H-pyran-2-carboxylic acid (Compound 218)

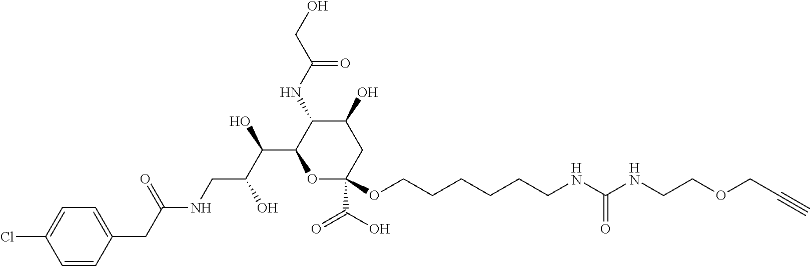 [SMILES: C#CCOCCNC(=O)NCCCCCCO[C@]1(C(=O)O)C[C@H](O)[C@@H](NC(=O)CO)[C@H]([C@H](O)[C@H](O)CNC(=O)Cc2ccc(Cl)cc2)O1]

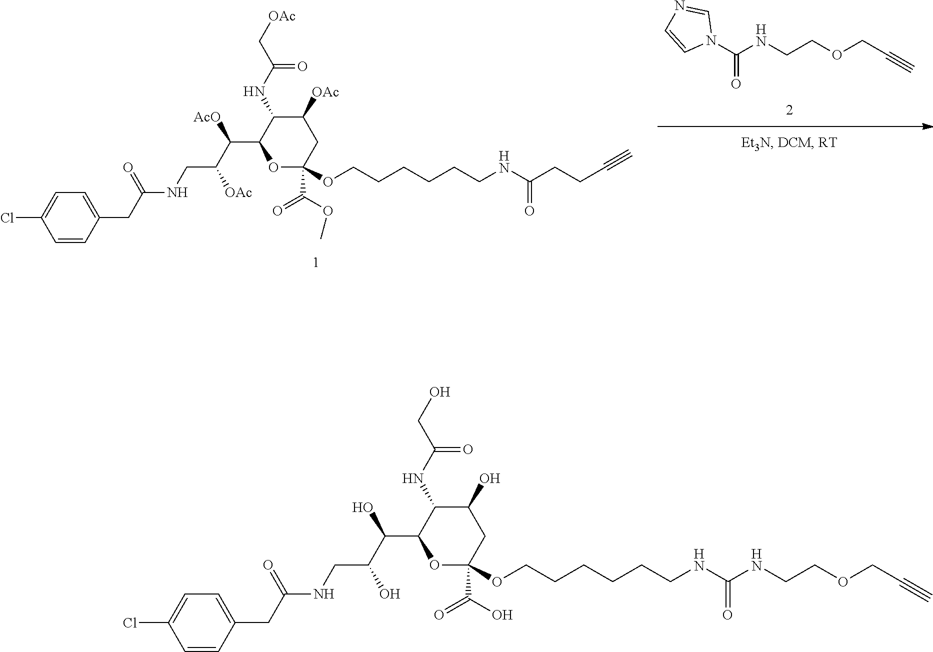 [SMILES: C#CCCC(=O)NCCCCCCO[C@]1(C(=O)OC)C[C@H](OC(C)=O)[C@@H](NC(=O)COC(C)=O)[C@H]([C@H](OC(C)=O)[C@@H](CNC(=O)Cc2ccc(Cl)cc2)OC(C)=O)O1 ; C#CCOCCNC(=O)n1ccnc1 ; C#CCOCCNC(=O)NCCCCCCO[C@]1(C(=O)O)C[C@H](O)[C@@H](NC(=O)CO)[C@H]([C@H](O)[C@H](O)CNC(=O)Cc2ccc(Cl)cc2)O1]

### Synthesis of (2R,4S,5R,6R)-6-((1R,2R)-3-(2-(4-chlorophenyl)acetamido)-1,2-dihydroxypropyl)-4-hydroxy-5-(2-hydroxyacetamido)-2-((6-(3-(2-(prop-2-yn-1-yloxy)ethyl)ureido)hexyl)oxy)tetrahydro-2H-pyran-2-carboxylic acid (Cpd. 218)

To a stirred solution of (1R,2R)-1-((2R,3R,4S,6R)-4-acetoxy-3-(2-acetoxyacetamido)-6-(methoxycarbonyl)-6-((6-(pent-4-ynamido)hexyl)oxy)tetrahydro-2H-pyran-2-yl)-3-(2-(4-chlorophenyl)acetamido)propane-1,2-diyl diacetate (1, 0.20 g, 0.347 mmol) in N,N-dimethylformamide (4 mL) were added triethylamine (0.097 mL, 0.694 mmol) and N-(2-(prop-2-yn-1-yloxy)ethyl)-1H-imidazole-1-carboxamide (2, 0.080 g, 0.417 mmol) at room temperature. The reaction mixture was stirred at room temperature for 24 h. After completion the reaction mixture was concentrated. The crude residue obtained was purified by prep HPLC using acetonitrile in water (+0.1% TFA) to afford the desired product (2R,4S,5R,6R)-6-((1R,2R)-3-(2-(4-chlorophenyl)acetamido)-1,2-dihydroxypropyl)-4-hydroxy-5-(2-hydroxyacetamido)-2-((6-(3-(2-(prop-2-yn-1-yloxy)ethyl)ureido)hexyl)oxy)tetrahydro-2H-pyran-2-carboxylic acid (Cpd. No. Cpd. 218). Yield: 0.014 g, 6%; LCMS m/z 70.1.5 [M+H]+; ¹H NMR (400 MHz, Methanol-d₄): δ 7.33-7.28 (m, 4H), 4.17 (d, J=2.4 Hz, 2H), 4.05-4.03 (m, 2H), 3.91-3.65 (m, 7H), 3.57-3.54 (m, 4H), 3.42-3.32 (m, 2H), 3.25-3.21 (m, 2H), 3.12-3.09 (m, 2H), 2.85-2.8 (m, 1H), 2.74-2.70 (dd, J=3.6 Hz, 12.0 Hz, 1H), 1.73 (t, J=11.4 Hz, 1H), 1.62-1.51 (m, 4H), 1.48-1.35 (m, 4H).

### (2R,4S,5R,6R)-5-(2-amino-2-oxoacetamido)-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (Compound 223)

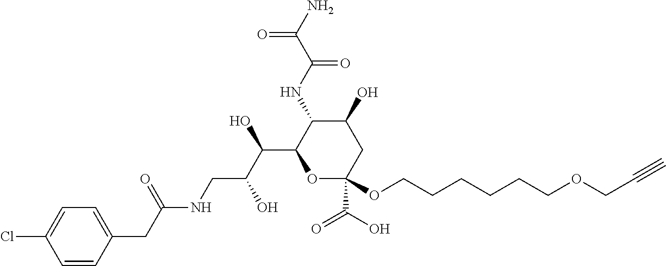 [SMILES: C#CCOCCCCCCO[C@]1(C(=O)O)C[C@H](O)[C@@H](NC(=O)C(N)=O)[C@H]([C@H](O)[C@H](O)CNC(=O)Cc2ccc(Cl)cc2)O1]

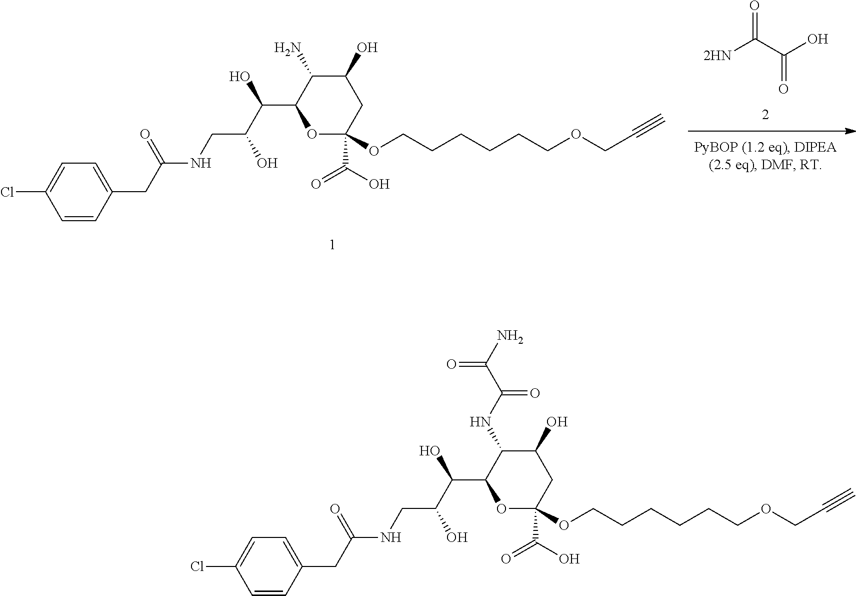

### Synthesis of (2R,4S,5R,6R)-5-(2-amino-2-oxoacetamido)-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (Cpd. 223)

To a stirred solution of (2R,4S,5R,6R)-6-[(1R,2R)-3-{[(p-chlorophenyl)methyl]carbonylamino}-1,2-dihydroxypropyl]-5-amino-4-hydroxy-2-[6-(2-propynyloxy)hexyloxy]tetrahydro-2H-pyran-2-carboxylic acid (1, 0.10 g, 0.180 mmol) and oxamic acid (2, 0.016 g, 0.180 mmol) in N,N dimethylformamide (3 mL) were added 1H-1,2,3-benzotriazol-1-yloxy-tri-1-pyrrolidinylphosphonium hexafluoridophosphate(1-) (0.112 g, 0.215 mmol) and ethylbis(propan-2-yl)amine (0.058 mL, 0.449 mmol) sequentially at 0° C. and reaction mixture was stirred at room temperature for 16 h. After completion the reaction mixture was concentrated. The crude residue obtained was purified by prep HPLC using acetonitrile in water (+0.1% TFA) to afford the desired product (2R,4S,5R,6R)-5-(2-amino-2-oxoacetamido)-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (Cpd. 223). Yield: 0.0035 g, 3.1%; LCMS m/z 628.40 [M+H]+; ¹H NMR (400 MHz, Methanol-d₄): δ 7.31-7.25 (m, 4H), 4.12 (d, J=2.4 Hz, 2H), 3.89-3.72 (m, 5H), 3.64-3.60 (m, 1H), 3.52-3.49 (m, 4H), 3.44-3.36 (m, 2H), 3.25-3.24 (m, 1H), 2.80 (t, J=3.6 Hz, 1H), 2.75-2.71 (dd, J=3.6 Hz, 12.0 Hz, 1H), 1.69 (t, J=12.0 Hz, 1H), 1.57-1.54 (m, 4H), 1.40-1.36 (m, 4H).

### (2R,4S,5R,6R)-6-((1R,2R)-3-(2-(4-(1H-tetrazol-5-yl)phenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Compound 176)

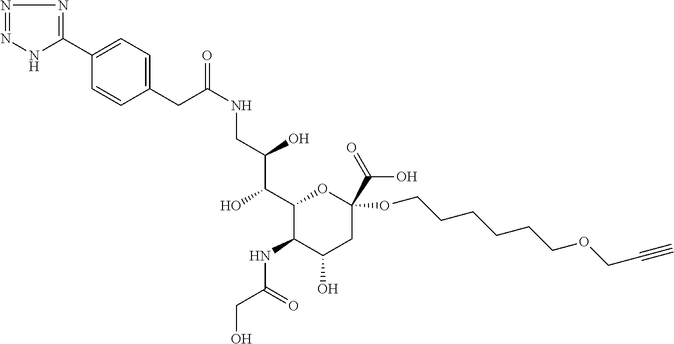 [SMILES: C#CCOCCCCCCO[C@]1(C(=O)O)C[C@H](O)[C@@H](NC(=O)CO)[C@H]([C@H](O)[C@H](O)CNC(=O)Cc2ccc(-c3nnn[nH]3)cc2)O1]

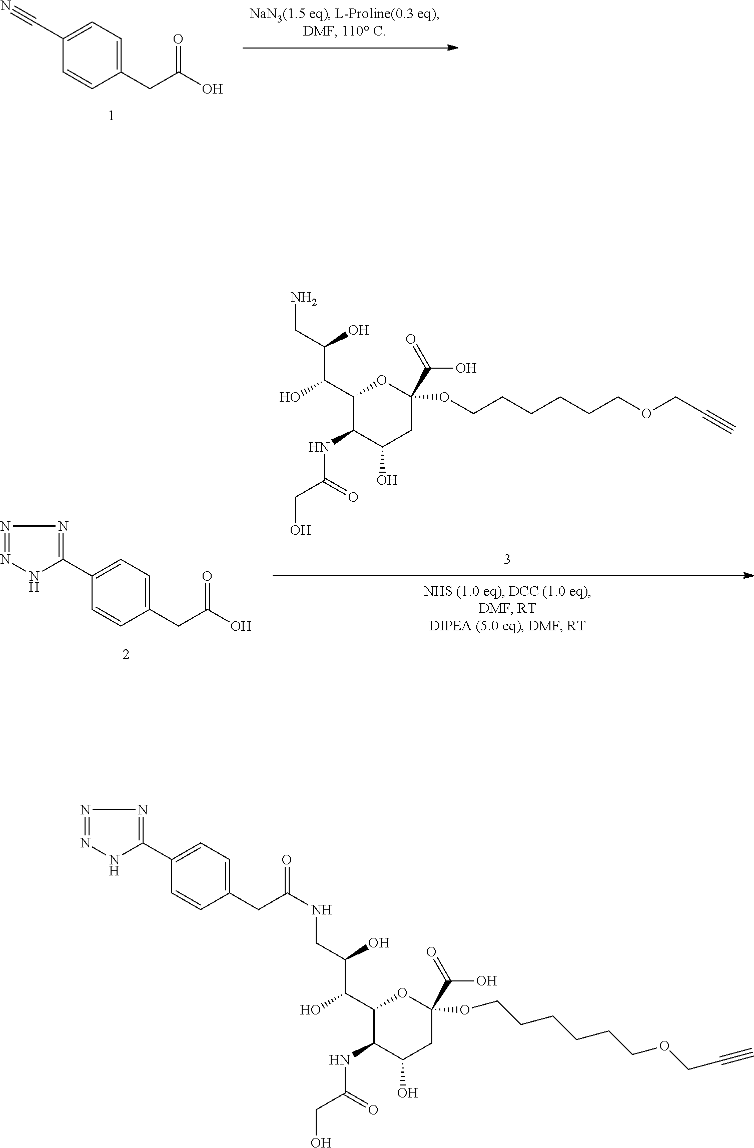 [SMILES: N#Cc1ccc(CC(=O)O)cc1 ; O=C(O)Cc1ccc(-c2nnn[nH]2)cc1 ; C#CCOCCCCCCO[C@]1(C(=O)O)C[C@H](O)[C@@H](NC(=O)CO)[C@H]([C@H](O)[C@H](O)CN)O1 ; C#CCOCCCCCCO[C@]1(C(=O)O)C[C@H](O)[C@@H](NC(=O)CO)[C@H]([C@H](O)[C@H](O)CNC(=O)Cc2ccc(-c3nnn[nH]3)cc2)O1]

### Synthesis of 2-(4-(1H-tetrazol-5-yl)phenyl)acetic acid (2)

To a stirred solution of (p-cyanophenyl)acetic acid (1, 0.5 g, 3.10 mmol) in dimethylformamide (5 mL) were added sodium azide (0.262 g, 4.03 mmol) and L-proline (0.107 g, 0.931 mmol), and the mixture was stirred at 110° C. for 10 h. After completion, the reaction mixture was allowed to cool down to room temperature then poured in ice water under stirring. The resulting mixture was acidified with diluted HCl under vigorous stirring. The precipitate formed was collected by filtration and rinsed with cold water, then air dried to afford the desired product [p-(1H-tetraazol-5-yl)phenyl]acetic acid (2). Yield: 0.250 g, 39%; LCMS (ESI) m/z 205.07 [M+H]⁺.

### Synthesis of (2R,4S,5R,6R)-6-((1R,2R)-3-(2-(4-(1H-tetrazol-5-yl)phenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (Cpd. 176)

To a stirred solution of [p-(1H-tetraazol-5-yl)phenyl]acetic acid (2, 0.1 g, 0.49 mmol) in DMF (2 mL), 1-[(cyclohexylimino)methyleneamino]cyclohexane (0.152 g, 0.73 mmol) was added and stirred for 10 min. 1-hydroxy-2,5-pyrrolidinedione (0.042 g, 0.37 mmol) was added and stirred for 4 h. After completion, the mixture was concentrated, and the crude residue obtained was used for the next step. A solution of (2R,4S,5R,6R)-6-[(1R,2R)-3-amino-1,2-dihydroxypropyl]-4-hydroxy-5-[(hydroxymethyl)carbonylamino]-2-[6-(2-propynyloxy)hexyloxy]tetrahydro-2H-pyran-2-carboxylic acid (3, 0.227 g, 0.49 mmol) and N-ethylbis(isopropyl)amine (0.42 mL, 2.45 mmol) in DMF (2 mL) was added at 0° C., was stirred for 10 h. After completion the solvent was concentrated material which was purified by prep HPLC using acetonitrile in water (+0.1% TFA) as eluents to afford the desired product (2R,4S,5R,6R)-6-[(1R,2R)-1,2-dihydroxy-3-({[p-(1H-tetraazol-5-yl)phenyl]methyl}carbonylamino)propyl]-4-hydroxy-5-[(hydroxymethyl)carbonylamino]-2-[6-(2-propynyloxy)hexyloxy]tetrahydro-2H-pyran-2-carboxylic acid (Cpd. 176). Yield: 0.023 g, 7%; LCMS m/z 649.28 [M+H]+; ¹H-NMR (400 MHz, methanol-d₄): δ 8.13 (bs, 1H), 7.97 (d, J=8.4 Hz, 2H), 7.53 (d, J=8.0 Hz, 2H), 4.12-4.10 (m, 2H), 4.00 (s, 2H), 3.92-3.68 (m, 6H), 3.65 (s, 2H), 3.56-3.50 (m, 3H), 3.47-3.38 (m, 1H), 3.36-3.27 (m, 1H), 2.81-2.80 (m, 1H), 2.76-2.69 (dd, J=12.4 Hz & 3.6 Hz, 1H), 1.69 (t, J=12.0 Hz, 1H), 1.60-1.53 (m, 4H), 1.39-1.34 (m, 4H).

### (2R,4S,5R,6R)-6-((1R,2R)-3-(2-(4-(1H-tetrazol-5-yl)phenyl)acetamido)-1,2-dihydroxypropyl)-2-((6-((1-(18-(2,5-dioxo-2,5-dihydro-1H-pyrrol-1-yl)-15-oxo-3,6,9,12-tetraoxa-16-azaoctadecyl)-1H-1,2,3-triazol-4-yl)methoxy)hexyl)oxy)-4-hydroxy-5-(2-hydroxyacetamido)tetrahydro-2H-pyran-2-carboxylic acid (Compound 176M)

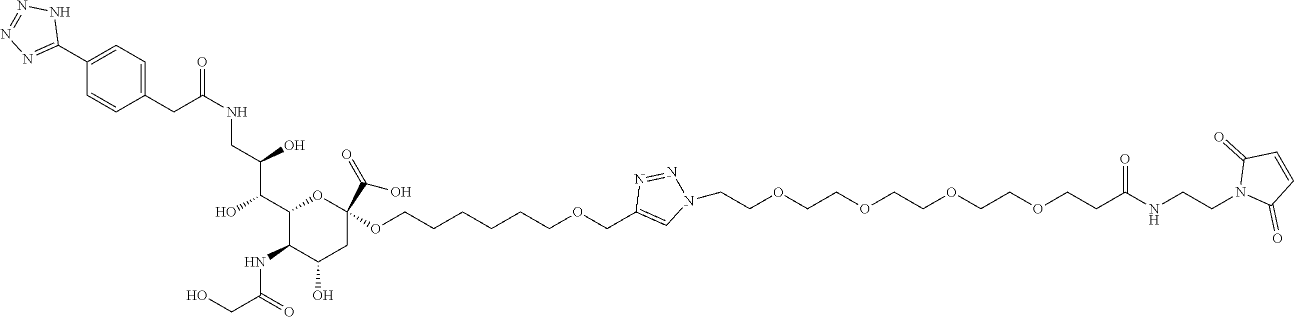 [SMILES: O=C(CCOCCOCCOCCOCCn1cc(COCCCCCCO[C@]2(C(=O)O)C[C@H](O)[C@@H](NC(=O)CO)[C@H]([C@H](O)[C@H](O)CNC(=O)Cc3ccc(-c4nnn[nH]4)cc3)O2)nn1)NCCN1C(=O)C=CC1=O]

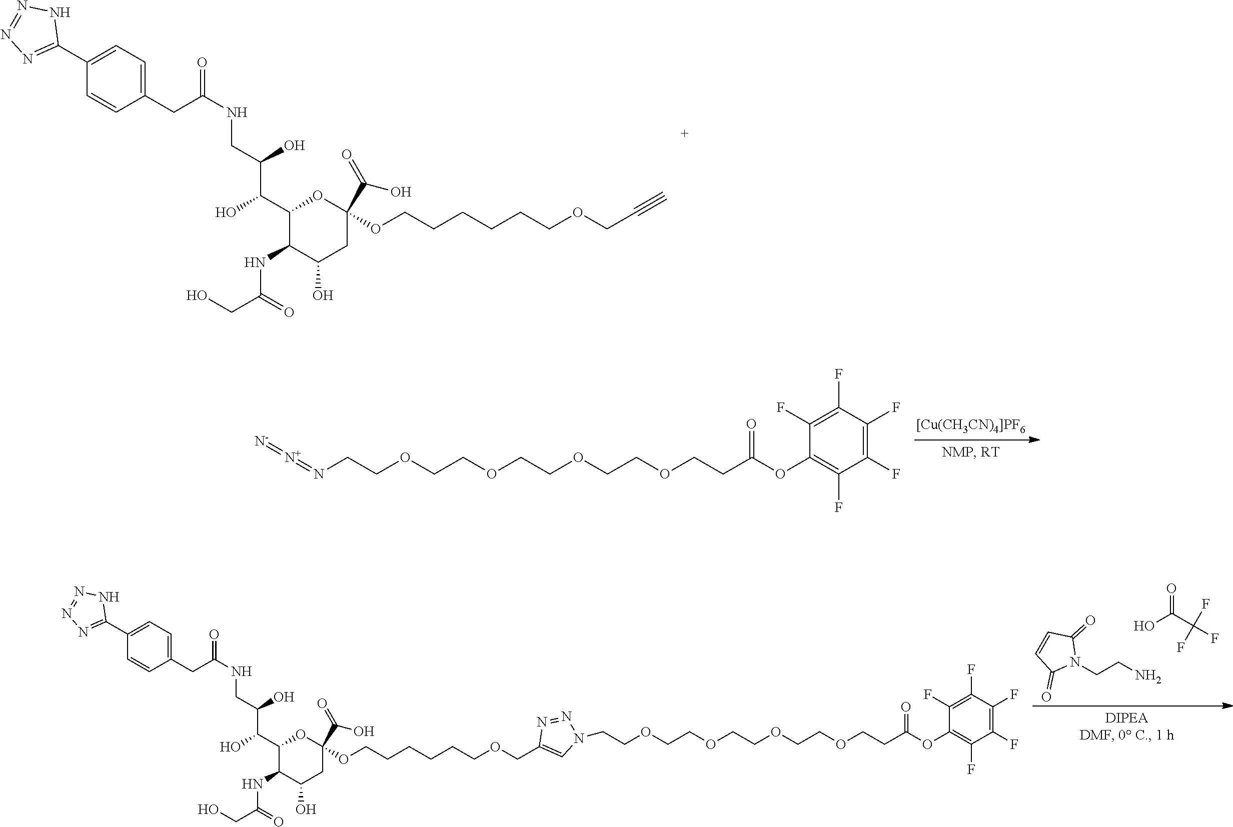

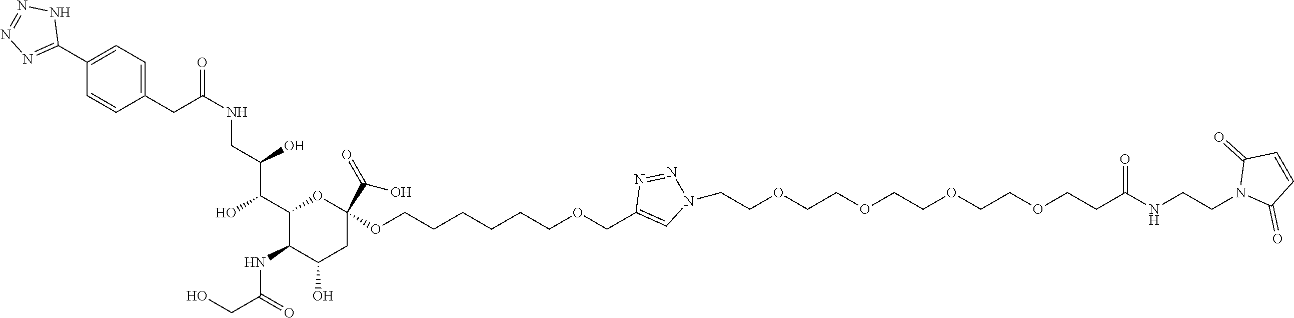

### Synthesis of (2R,4S,5R,6R)-6-((1R,2R)-3-(2-(4-(1H-tetrazol-5-yl)phenyl)acetamido)-1,2-dihydroxypropyl)-4-hydroxy-5-(2-hydroxyacetamido)-2-((6-((1-(15-oxo-15-(perfluorophenoxy)-3,6,9,12-tetraoxapentadecyl)-1H-1,2,3-triazol-4-yl)methoxy)hexyl)oxy)tetrahydro-2H-pyran-2-carboxylic acid (176P)

To a solution of (2R,4S,5R,6R)-6-((1R,2R)-3-(2-(4-(1H-tetrazol-5-yl)phenyl)acetamido)-1,2-dihydroxypropyl)-4-hydroxy-5-(2-hydroxyacetamido)-2-((6-(prop-2-yn-1-yloxy)hexyl)oxy)tetrahydro-2H-pyran-2-carboxylic acid (176, 18.3 mg, 28.2 umol) in anhydrous NMP (182 uL) was added Azido-PEG4-PFP-ester (15.5 mg, 33.9 umol) in anhydrous NMP (182 uL) dropwise, followed by Tetrakis(acetonitrile)copper(l) hexafluorophosphate (15.8 mg, 42.3 umol). The vial was sealed and the mixture was stirred for 45 min at room temperature. The mixture was filtered through a 0.2 μm PTFE filter, a few drops of acetic acid were added, and the filtrate was purified by C18 reverse phase column chromatography using acetonitrile and water (+0.1% TFA) as eluents. The fractions containing the desired product were lyophilized to afford 116P as a white solid. Yield: 13.4 mg, 43%; LCMS (ESI) m/z 1106 [M+H]⁺.

### Synthesis of (2R,4S,5R,6R)-6-((1R,2R)-3-(2-(4-(1H-tetrazol-5-yl)phenyl)acetamido)-1,2-dihydroxypropyl)-2-((6-((1-(18-(2,5-dioxo-2,5-dihydro-1H-pyrrol-1-yl)-15-oxo-3,6,9,12-tetraoxa-16-azaoctadecyl)-1H-1,2,3-triazol-4-yl)methoxy)hexyl)oxy)-4-hydroxy-5-(2-hydroxyacetamido)tetrahydro-2H-pyran-2-carboxylic acid (176M)

To a solution of (2R,4S,5R,6R)-6-((1R,2R)-3-(2-(4-(1H-tetrazol-5-yl)phenyl)acetamido)-1,2-dihydroxypropyl)-4-hydroxy-5-(2-hydroxyacetamido)-2-((6-((1-(15-oxo-15-(perfluorophenoxy)-3,6,9,12-tetraoxapentadecyl)-1H-1,2,3-triazol-4-yl)methoxy)hexyl)oxy)tetrahydro-2H-pyran-2-carboxylic acid (176P, 13.1 mg, 11.8 umol) in DMF (296 uL) was added n-(2-aminoethyl)maleimide trifluoroacetate salt (3.16 mg, 12.4 umol). The mixture was cooled down to 0° C. and stirred for 10 min. DIEA (6.19 uL, 35.5 umol) was added and the mixture was stirred for 1 h at 0° C. The reaction mixture was filtered and purified by preparatory HPLC using acetonitrile and water (+0.05% TFA) as eluents. The fractions containing the desired product were combined and lyophilized to dryness to afford the 176M as a white solid. Yield: 9.37 mg, 74%; LCMS (ESI) m/z 1062.8 [M+H]⁺. ¹H NMR (500 MHz, DMSO-d₆): 8.06 (t, J=5.5 Hz, 1H), 8.03 (s, 1H), 7.95 (d, J=8.0 Hz, 2H), 7.93 (t, J=5.5 Hz, 1H), 7.85 (d, J=8.0 Hz, 1H), 7.48 (d, J=8.0 Hz, 2H), 6.99 (s, 2H), 4.50 (t, J=5.0 Hz, 2H), 4.45 (s, 2H), 3.87 (m, 2H), 3.83-3.81 (m, 1H), 3.80 (t, J=5.0 Hz, 2H), 3.70-3.66 (m, 1H), 3.62-3.59 (m, 1H), 3.57-3.50 (m, 9H), 3.47-3.39 (m, 18H), 3.29-3.26 (m, 2H), 3.22-3.16 (m, 4H), 2.99-2.94 (m, 1H), 2.21 (t, J=6.5 Hz, 2H), 1.52-1.49 (m, 1H), 1.48-1.42 (m, 4H), 1.25-1.23 (m, 4H).

### (2R,4S,5R,6R)-6-((1R,2R)-3-(2-(4-(difluoromethyl)phenyl)acetamido)-1,2-dihydroxypropyl)-4-hydroxy-2-((6-((hydroxy(2-(prop-2-yn-1-yloxy)ethoxy)phosphoryl)oxy)hexyl)oxy)-5-(2-hydroxyacetamido)tetrahydro-2H-pyran-2-carboxylic acid (Compound 215)

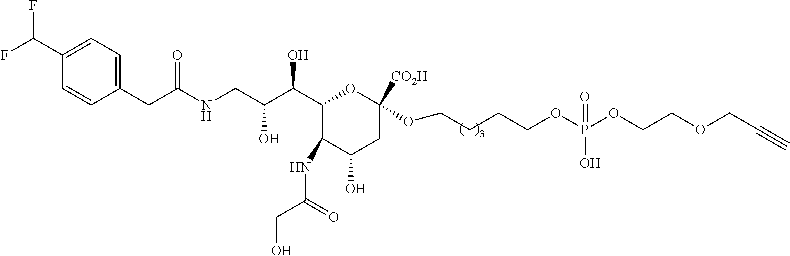

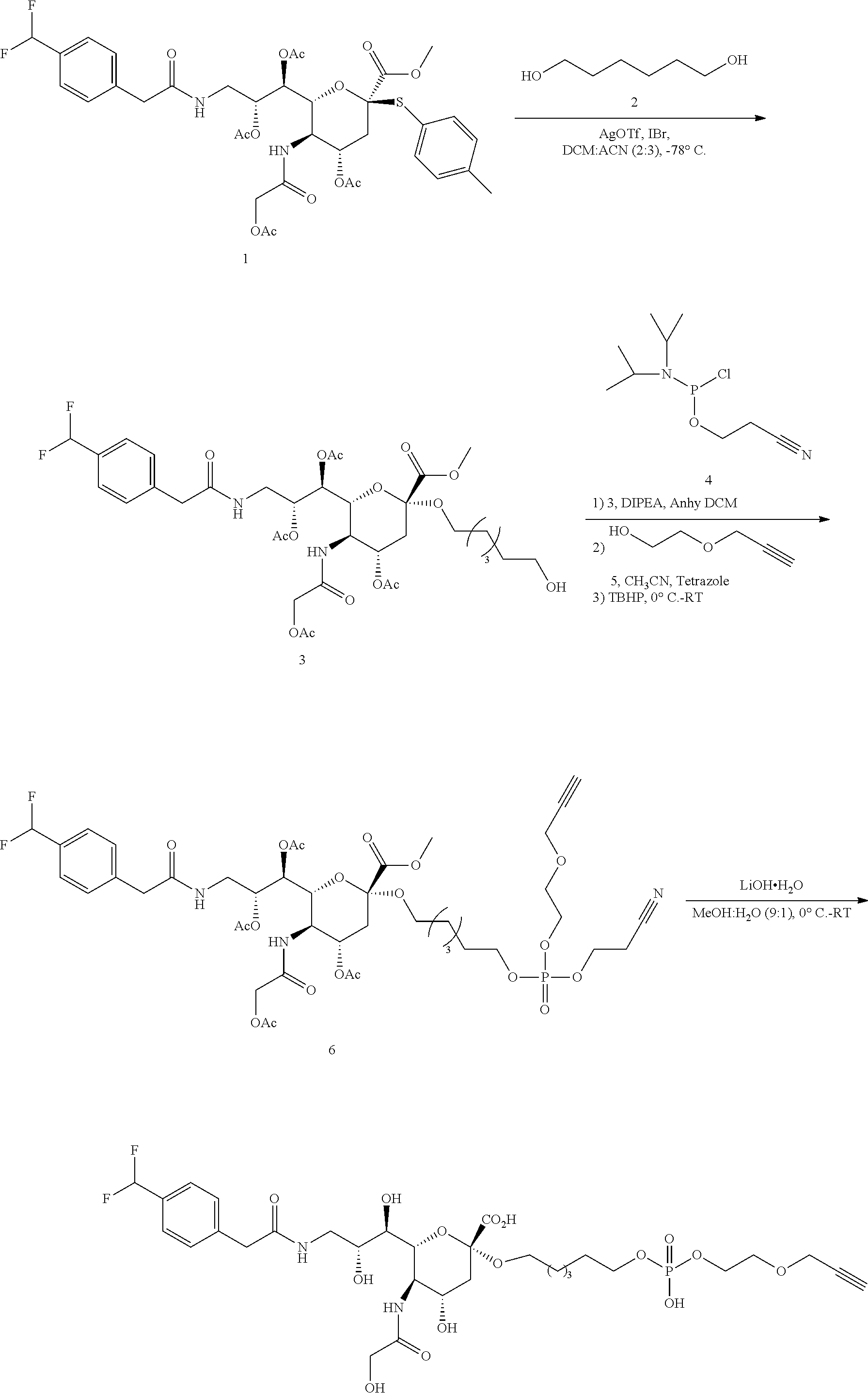

### Synthesis of (1R,2R)-1-((2R,3R,4S,6R)-4-acetoxy-3-(2-acetoxyacetamido)-6-((6-hydroxyhexyl)oxy)-6-(methoxycarbonyl)tetrahydro-2H-pyran-2-yl)-3-(2-(4-(difluoromethyl)phenyl)acetamido)propane-1,2-diyl diacetate (3)

A mixture of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-(difluoromethyl)phenyl)acetamido)propane-1,2-diyl diacetate (1, 0.20 g, 0.26 mmol), hexane-1,6-diol (2, 0.30 g, 2.56 mmol), silver trifluoromethanesulfonate (0.26 g, 1.02 mmol) and activated 4 Å powdered molecular sieves (0.2 g) in anhydrous dichloromethane (20.0 mL) and acetonitrile (30.0 mL) was stirred at room temperature for 2 h. A solution of Iodine mono bromide (0.13 g, 0.64 mmol) in dichloromethane (5.0 mL) was added at −78° C. then stirred at the same temperature for 2 h. After completion, the reaction mixture was quenched by addition of triethyl amine (0.2 mL) and warmed to room temperature. The reaction mixture was filtered through celite. The filtrate was washed with a saturated solution of sodium thiosulphate and dried, filtered, and concentrated. The crude residue obtained was purified by silica gel column chromatography using ethyl acetate in heptane to afford the desired product (1R,2R)-1-((2R,3R,4S,6R)-4-acetoxy-3-(2-acetoxyacetamido)-6-((6-hydroxyhexyl)oxy)-6-(methoxycarbonyl)tetrahydro-2H-pyran-2-yl)-3-(2-(4-(difluoromethyl)phenyl)acetamido)propane-1,2-diyl diacetate (3). Yield: 0.1 g, 52%; LCMS (ESI) m/z 775.70 [M+H]⁺.

### Synthesis of (1R,2R)-1-((2R,3R,4S,6R)-4-acetoxy-3-(2-acetoxyacetamido)-6-((6-(((2-cyanoethoxy)(2-(prop-2-yn-1-yloxy)ethoxy)phosphoryl)oxy)hexyl)oxy)-6-(methoxycarbonyl)tetrahydro-2H-pyran-2-yl)-3-(2-(4-(difluoromethyl)phenyl)acetamido)propane-1,2-diyl diacetate (5)

To a stirred solution of 3-((chloro(diisopropylamino)phosphaneyl)oxy)propanenitrile (4, 0.097 g, 0.41 mmol) in dichloromethane (4 ml) was added ethylbis(propan-2-yl)amine (0.11 mL, 0.615 mmol) was added dropwise at 0° C. then stirred at same temperature for 10 min, then (1R,2R)-1-((2R,3R,4S,6R)-4-acetoxy-3-(2-acetoxyacetamido)-6-((6-hydroxyhexyl)oxy)-6-(methoxycarbonyl)tetrahydro-2H-pyran-2-yl)-3-(2-(4-(difluoromethyl)phenyl)acetamido)propane-1,2-diyl diacetate (3, 0.05 g, 0.065 mmol) in dichloromethane (1 mL) was added to the reaction mixture was stirred for another 30 min at room temperature. After completion 2-(prop-2-yn-1-yloxy)ethan-1-ol (5, 0.01 g, 0.10 mmol) and tetrazole (0.011 g, 0.10 mmol) in acetonitrile (0.5 mL) were added sequentially at 0° C. was stirred for another 1 h at room temperature. After completion, tert-butyl hydroperoxide (0.5 mL) was added at 0° C. then stirred at room temperature for 1 h. After completion, the reaction mixture was concentrated to afford the desired crude product (1R,2R)-1-((2R,3R,4S,6R)-4-acetoxy-3-(2-acetoxyacetamido)-6-((6-(((2-cyanoethoxy)(2-(prop-2-yn-1-yloxy)ethoxy)phosphoryl)oxy)hexyl)oxy)-6-(methoxycarbonyl)tetrahydro-2H-pyran-2-yl)-3-(2-(4-(difluoromethyl)phenyl)acetamido)propane-1,2-diyl diacetate (6) as a crude which was used for the next step without further purification. Yield: 0.05 g, crude; LCMS m/z 990.85 [M+H]⁺.

### Synthesis of (2R,4S,5R,6R)-6-((1R,2R)-3-(2-(4-(difluoromethyl)phenyl)acetamido)-1,2-dihydroxypropyl)-4-hydroxy-2-((6-((hydroxy(2-(prop-2-yn-1-yloxy)ethoxy)phosphoryl)oxy)hexyl)oxy)-5-(2-hydroxyacetamido)tetrahydro-2H-pyran-2-carboxylic acid (Cpd. 215)

To the stirred solution of (1R,2R)-1-((2R,3R,4S,6R)-4-acetoxy-3-(2-acetoxyacetamido)-6-((6-(((2-cyanoethoxy)(2-(prop-2-yn-1-yloxy)ethoxy)phosphoryl)oxy)hexyl)oxy)-6-(methoxycarbonyl)tetrahydro-2H-pyran-2-yl)-3-(2-(4-(difluoromethyl)phenyl)acetamido)propane-1,2-diyl diacetate (6, 0.12 g, 0.12 mmol) in methanol (5.0 mL) and water (0.5 mL), was added lithium hydroxide monohydrate (0.017 g, 0.72 mmol) at 0° C. The reaction mixture was stirred at room temperature for 3 h. After completion, the reaction mixture was quenched with Dowex-H⁺ resin and filtered. The filtrate was concentrated. The crude was purified by prep-HPLC using acetonitrile in water (+0.1% TFA) as eluents to afford the desired product (2R,4S,5R,6R)-6-((1R,2R)-3-(2-(4-(difluoromethyl)phenyl)acetamido)-1,2-dihydroxypropyl)-4-hydroxy-2-((6-((hydroxy(2-(prop-2-yn-1-yloxy)ethoxy)phosphoryl)oxy)hexyl)oxy)-5-(2-hydroxyacetamido)tetrahydro-2H-pyran-2-carboxylic acid (Cpd. 215). Yield: 0.007 g, 7.65%; LCMS m/z 755.50 [M+H]+; ¹H-NMR (400 MHz, Methanol-d₄): δ 7.49-7.42 (m, 4H), 6.74 (t, J=16.4 Hz, 1H), 4.19 (d, J=2.0 Hz, 2H), 4.00-3.99 (m, 4H), 3.90-3.87 (m, 3H), 3.80-3.76 (m, 3H), 3.71-3.61 (m, 6H), 3.45-3.41 (m, 1H), 3.37-3.31 (m, 1H), 3.27-3.22 (m, 1H), 2.82 (t, J=2.8 Hz, 1H), 2.73 (dd, J=12.4 Hz & 4.0 Hz, 1H), 1.73-1.55 (m, 5H), 1.40-1.30 (m, 4H).

### (2R,4S,5R,6R)-6-((1R,2R)-3-(2-(4-(difluoromethyl)phenyl)acetamido)-1,2-dihydroxypropyl)-2-((6-((3,4-dioxo-2-((2-(prop-2-yn-1-yloxy)ethyl)amino)cyclobut-1-en-1-yl)amino)hexyl)oxy)-4-hydroxy-5-(2-hydroxyacetamido)tetrahydro-2H-pyran-2-carboxylic acid (Compound 216)

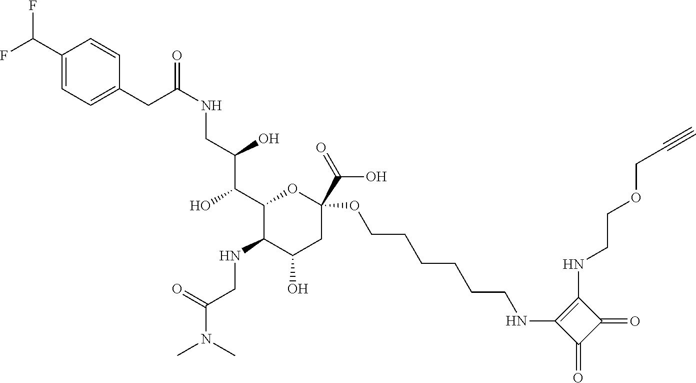 [SMILES: C#CCOCCNc1c(NCCCCCCO[C@]2(C(=O)O)C[C@H](O)[C@@H](NCC(=O)N(C)C)[C@H]([C@H](O)[C@H](O)CNC(=O)Cc3ccc(C(F)F)cc3)O2)c(=O)c1=O]

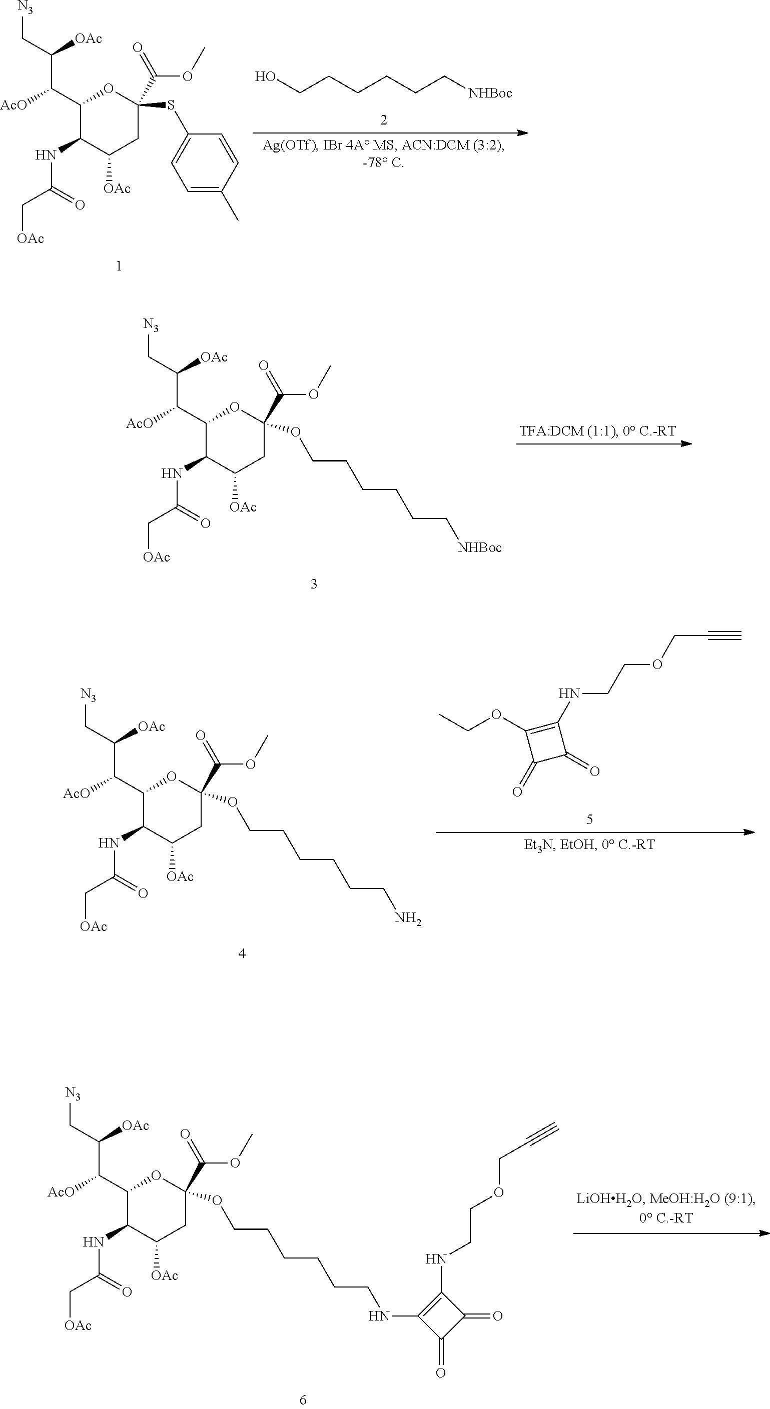

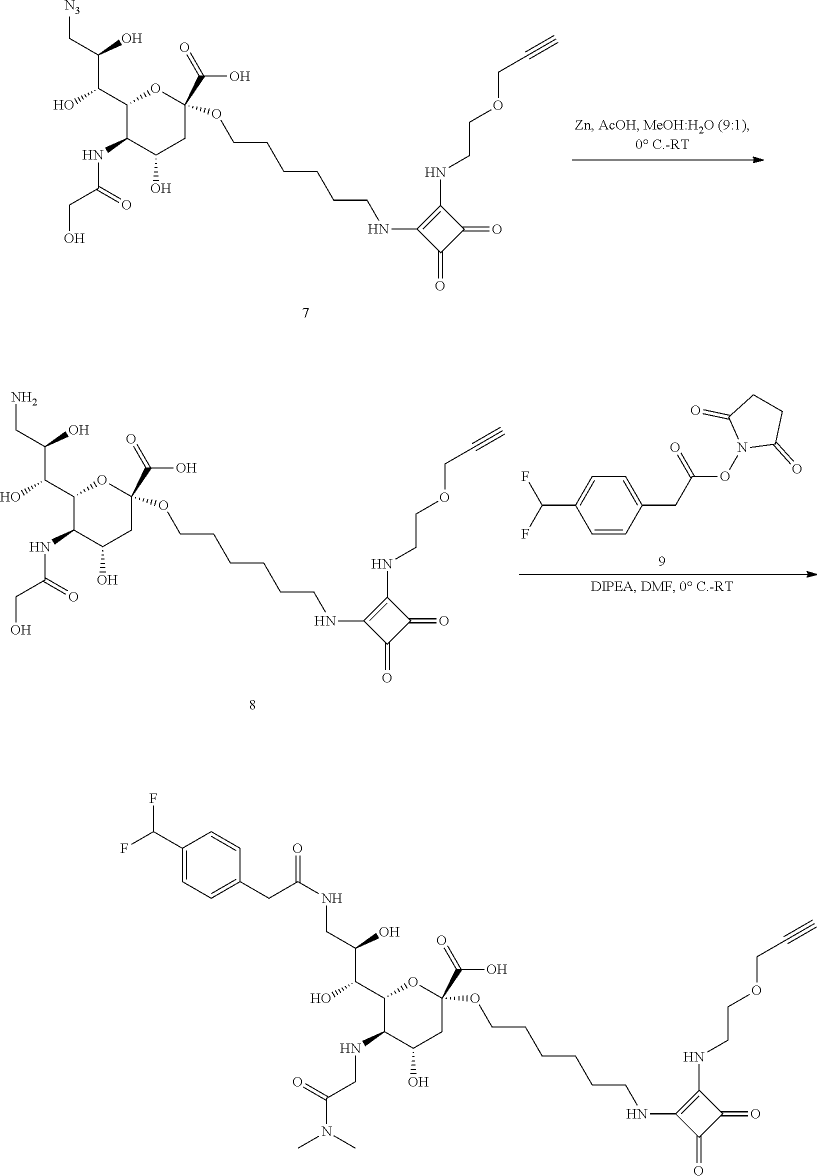 [SMILES: C#CCOCCNc1c(NCCCCCCO[C@]2(C(=O)O)C[C@H](O)[C@@H](NC(=O)CO)[C@H]([C@H](O)[C@H](O)CN=[N+]=[N-])O2)c(=O)c1=O ; C#CCOCCNc1c(NCCCCCCO[C@]2(C(=O)O)C[C@H](O)[C@@H](NC(=O)CO)[C@H]([C@H](O)[C@H](O)CN)O2)c(=O)c1=O ; O=C(Cc1ccc(C(F)F)cc1)ON1C(=O)CCC1=O ; C#CCOCCNc1c(NCCCCCCO[C@]2(C(=O)O)C[C@H](O)[C@@H](NCC(=O)N(C)C)[C@H]([C@H](O)[C@H](O)CNC(=O)Cc3ccc(C(F)F)cc3)O2)c(=O)c1=O]

### Synthesis of (1R,2R)-1-((2R,3R,4S,6R)-4-acetoxy-3-(2-acetoxyacetamido)-6-((6-((tert-butoxycarbonyl)amino)hexyl)oxy)-6-(methoxycarbonyl)tetrahydro-2H-pyran-2-yl)-3-azidopropane-1,2-diyl diacetate (3)

A mixture of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-azidopropane-1,2-diyl diacetate (1, 1.0 g, 1.57 mmol), tert-butyl (6-hydroxyhexyl)carbamate (2, 1.7 g, 7.83 mmol), silver trifluoromethanesulfonate (1.01 g, 3.91 mmol) and activated 4 Å powdered molecular sieves (0.5 g) in anhydrous dichloromethane (35.0 mL) and acetonitrile (60.0 mL) was stirred at room temperature for 2 h. The solution was cooled to −78° C. and bromoiodane (0.48 g, 2.35 mmol) in dichloromethane (5.0 mL) was added dropwise to the reaction mixture and was stirred at same temperature for 1 h. After completion, the reaction mixture was quenched by triethylamine (1.0 mL) and warmed to room temperature. The reaction mixture was filtered, washed by saturated solution of sodium bicarbonate, filtered, and concentrated. The crude residue obtained was purified by silica gel column chromatography using ethyl acetate in heptane to afford the desired product (1R,2R)-1-((2R,3R,4S,6R)-4-acetoxy-3-(2-acetoxyacetamido)-6-((6-((tert-butoxycarbonyl)amino)hexyl)oxy)-6-(methoxycarbonyl)tetrahydro-2H-pyran-2-yl)-3-azidopropane-1,2-diyl diacetate (3). Yield: 0.25 g, 21.82%; LCMS (ESI) m/z 729.75 [M−H]−.

### Synthesis of ((1R,2R)-1-((2R,3R,4S,6R)-4-acetoxy-3-(2-acetoxyacetamido)-6-((6-aminohexyl)oxy)-6-(methoxycarbonyl)tetrahydro-2H-pyran-2-yl)-3-azidopropane-1,2-diyl diacetate (4)

To a solution of methyl (1R,2R)-1-((2R,3R,4S,6R)-4-acetoxy-3-(2-acetoxyacetamido)-6-((6-((tert-butoxycarbonyl)amino)hexyl)oxy)-6-(methoxycarbonyl)tetrahydro-2H-pyran-2-yl)-3-azidopropane-1,2-diyl diacetate (3, 0.25 g, 0.34 mmol) in dichloromethane (5 mL), was added trifluoroacetic acid (5 mL) at 0° C. then stirred at room temperature for 4 h. After completion, the reaction mixture was concentrated to afford the desired product ((1R,2R)-1-((2R,3R,4S,6R)-4-acetoxy-3-(2-acetoxyacetamido)-6-((6-aminohexyl)oxy)-6-(methoxycarbonyl)tetrahydro-2H-pyran-2-yl)-3-azidopropane-1,2-diyl diacetate (4) as a crude which was used for the next step without further purification. Yield: 0.20 g, Crude; LCMS (ESI) m/z 631.80 [M+H]⁺.

### Synthesis of (1R,2R)-1-((2R,3R,4S,6R)-4-acetoxy-3-(2-acetoxyacetamido)-6-((6-((3,4-dioxo-2-((2-(prop-2-yn-1-yloxy)ethyl)amino)cyclobut-1-en-1-yl)amino)hexyl)oxy)-6-(methoxycarbonyl)tetrahydro-2H-pyran-2-yl)-3-azidopropane-1,2-diyl diacetate e (6)

To a stirred solution of (1R,2R)-1-((2R,3R,4S,6R)-4-acetoxy-3-(2-acetoxyacetamido)-6-((6-aminohexyl)oxy)-6-(methoxycarbonyl)tetrahydro-2H-pyran-2-yl)-3-azidopropane-1,2-diyl diacetate (4, 0.20 g, 0.31 mmol) and 3-ethoxy-4-((2-(prop-2-yn-1-yloxy)ethyl)amino)cyclobut-3-ene-1,2-dione (5, 0.11 g, 0.47 mmol) in ethanol (10.0 mL), triethylamine (0.13 mL, 0.96 mmol) was added in it at 0° C. and the reaction mixture was stirred for 2 h. After completion, the reaction mixture was concentrated, and triturated with mixture of ether and pentane to afford the desired product (1R,2R)-1-((2R,3R,4S,6R)-4-acetoxy-3-(2-acetoxyacetamido)-6-((6-((3,4-dioxo-2-((2-(prop-2-yn-1-yloxy)ethyl)amino)cyclobut-1-en-1-yl)amino)hexyl)oxy)-6-(methoxycarbonyl)tetrahydro-2H-pyran-2-yl)-3-azidopropane-1,2-diyl diacetate (6) as a crude which was used for the next step without further purification. Yield: 0.20 g, Crude; LCMS (ESI) m/z 808.70 [M+H]⁺.

### Synthesis of (2R,4S,5R,6R)-6-((1R,2R)-3-azido-1,2-dihydroxypropyl)-2-((6-((3,4-dioxo-2-((2-(prop-2-yn-1-yloxy)ethyl)amino)cyclobut-1-en-1-yl)amino)hexyl)oxy)-4-hydroxy-5-(2-hydroxyacetamido)tetrahydro-2H-pyran-2-carboxylic acid (7)

To a stirred solution of (1R,2R)-1-((2R,3R,4S,6R)-4-acetoxy-3-(2-acetoxyacetamido)-6-((6-((3,4-dioxo-2-((2-(prop-2-yn-1-yloxy)ethyl)amino)cyclobut-1-en-1-yl)amino)hexyl)oxy)-6-(methoxycarbonyl)tetrahydro-2H-pyran-2-yl)-3-azidopropane-1,2-diyl diacetate (6, 0.30 g, 0.37 mmol) in methanol (10.0 mL), lithium hydroxide monohydrate (0.18 g, 2.22 mmol) in water (1.0 mL) was added at 0° C. and the reaction mixture was stirred for 4 h at room temperature. The mixture was neutralized with acidic resin (Dowex 50, H⁺) to pH˜6 and the suspension was filtered. The filtrate was concentrated, and the crude residue obtained was purified by trituration to afford the desired product (2R,4S,5R,6R)-6-((1R,2R)-3-azido-1,2-dihydroxypropyl)-2-((6-((3,4-dioxo-2-((2-(prop-2-yn-1-yloxy)ethyl)amino)cyclobut-1-en-1-yl)amino)hexyl)oxy)-4-hydroxy-5-(2-hydroxyacetamido)tetrahydro-2H-pyran-2-carboxylic acid (7). Yield: 0.20 g, Crude; LCMS (ESI) m/z 626.80 [M+H]⁺.

### Synthesis of (2R,4S,5R,6R)-6-((1R,2R)-3-amino-1,2-dihydroxypropyl)-2-((6-((3,4-dioxo-2-((2-(prop-2-yn-1-yloxy)ethyl)amino)cyclobut-1-en-1-yl)amino)hexyl)oxy)-4-hydroxy-5-(2-hydroxyacetamido)tetrahydro-2H-pyran-2-carboxylic acid (8)

To a stirred solution of (2R,4S,5R,6R)-6-((1R,2R)-3-azido-1,2-dihydroxypropyl)-2-((6-((3,4-dioxo-2-((2-(prop-2-yn-1-yloxy)ethyl)amino)cyclobut-1-en-1-yl)amino)hexyl)oxy)-4-hydroxy-5-(2-hydroxyacetamido)tetrahydro-2H-pyran-2-carboxylic acid (7, 0.20 g, 0.32 mmol) in methanol (10.0 mL) and water (1.0 mL), zinc dust (0.21 g, 3.10 mmol) and acetic acid (0.09 mL, 1.60 mmol) was added portion wise at 0° C. and the reaction mixture was stirred at room temperature for 2 h. After completion, the reaction mixture was filtered. The filtrate was concentrated, and the crude residue obtained was purified by trituration with diethyl ether to afford the desired product (2R,4S,5R,6R)-6-((1R,2R)-3-amino-1,2-dihydroxypropyl)-2-((6-((3,4-dioxo-2-((2-(prop-2-yn-1-yloxy)ethyl)amino)cyclobut-1-en-1-yl)amino)hexyl)oxy)-4-hydroxy-5-(2-hydroxyacetamido)tetrahydro-2H-pyran-2-carboxylic acid (8) as a crude. Yield: 0.10 g, crude; LCMS m/z 600.85 [M+H]⁺;

### Synthesis of (2R,4S,5R,6R)-6-((1R,2R)-3-(2-(4-(difluoromethyl)phenyl)acetamido)-1,2-dihydroxypropyl)-2-((6-((3,4-dioxo-2-((2-(prop-2-yn-1-yloxy)ethyl)amino)cyclobut-1-en-1-yl)amino)hexyl)oxy)-4-hydroxy-5-(2-hydroxyacetamido)tetrahydro-2H-pyran-2-carboxylic acid (Cpd. 216)

To a stirred solution of (2R,4S,5R,6R)-6-((1R,2R)-3-amino-1,2-dihydroxypropyl)-2-((6-((3,4-dioxo-2-((2-(prop-2-yn-1-yloxy)ethyl)amino)cyclobut-1-en-1-yl)amino)hexyl)oxy)-4-hydroxy-5-(2-hydroxyacetamido)tetrahydro-2H-pyran-2-carboxylic acid (8, 0.15 g, 0.25 mmol) in N,N-dimethylformamide (3.0 mL), N,N-diisopropylethylamine (0.13 mL, 0.74 mmol) and perfluorophenyl 2-(4-chlorophenyl)acetate (9, 0.07 g, 0.25 mmol) was added at 0° C. and the reaction mixture was stirred at room temperature for 4 h. After completion, the reaction mixture was concentrated, and the crude residue obtained was purified by prep HPLC using acetonitrile in water (+0.1% TFA) as eluents to afford the desired product (2R,4S,5R,6R)-6-((1R,2R)-3-(2-(4-(difluoromethyl)phenyl)acetamido)-1,2-dihydroxypropyl)-2-((6-((3,4-dioxo-2-((2-(prop-2-yn-1-yloxy)ethyl)amino)cyclobut-1-en-1-yl)amino)hexyl)oxy)-4-hydroxy-5-(2-hydroxyacetamido)tetrahydro-2H-pyran-2-carboxylic acid (216). Yield: 0.012 g, 6.25%; LCMS m/z 769.50 [M+H]⁺. ¹H NMR (400 MHz, Methanol-d₄): δ 7.47 (d, J=8.0 Hz, 2H), 7.42 (d, J=8.4 Hz, 2H), 6.73 (t, J=16.4 Hz, 1H), 4.20 (d, J=2.4 Hz, 2H), 4.01 (s, 2H), 3.87 (td, J=8.4 & 3.2 Hz, 1H), 3.84-3.77 (m, 9H), 3.61-3.54 (m, 4H), 3.42-3.36 (m, 2H), 3.23-3.18 (m, 1H), 2.87 (t, J=2.4 Hz, 1H), 2.71 (dd, J=4.0 & 12.0 Hz, 1H), 1.71 (t, J=12.4 Hz, 1H), 1.59-1.54 (m, 4H), 1.40-1.33 (m, 4H).

### (2S,4S,5R,6R)-6-((1R,2R)-3-(2-(4-chlorophenyl)acetamido)-1,2-dihydroxypropyl)-4-hydroxy-5-(2-hydroxyacetamido)-2-((S)-1-hydroxyheptyl)tetrahydro-2H-pyran-2-carboxylic acid (Compound 203)

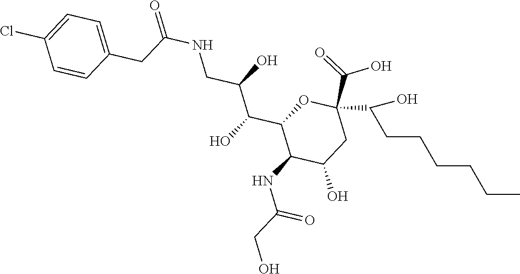 [SMILES: CCCCCCC(O)[C@]1(C(=O)O)C[C@H](O)[C@@H](NC(=O)CO)[C@H]([C@H](O)[C@H](O)CNC(=O)Cc2ccc(Cl)cc2)O1]

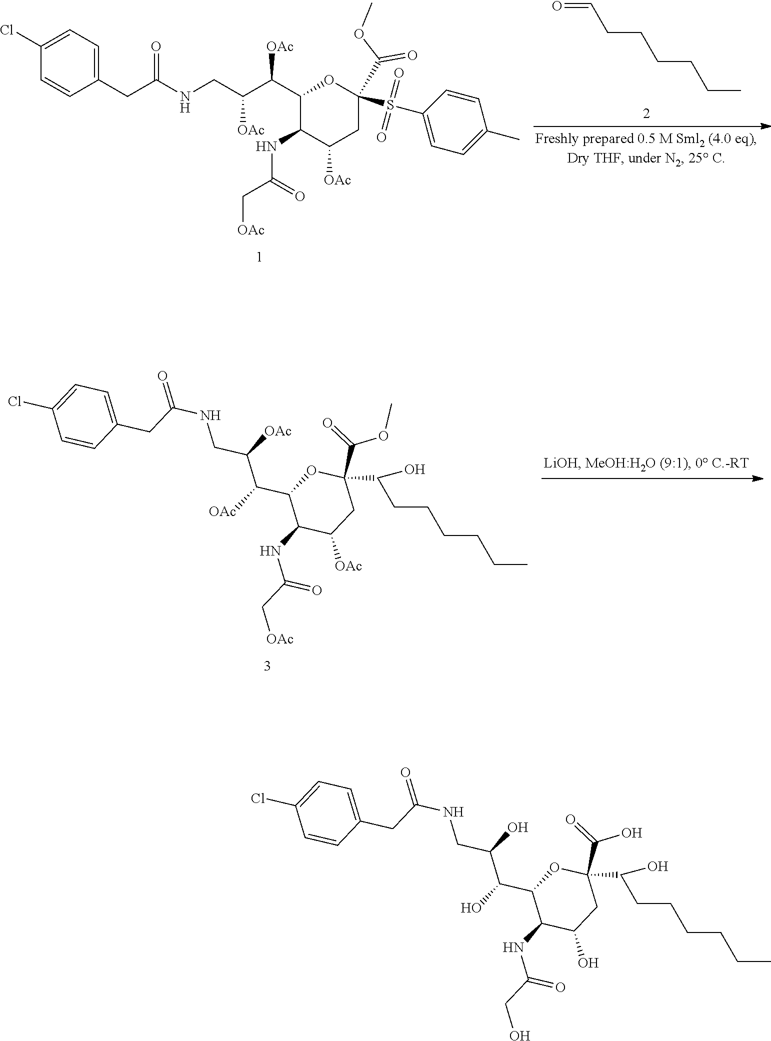

### Synthesis of (1R,2R)-1-((2R,3R,4S,6S)-4-acetoxy-3-(2-acetoxyacetamido)-6-((S)-1-hydroxyheptyl)-6-(methoxycarbonyl)tetrahydro-2H-pyran-2-yl)-3-(2-(4-chlorophenyl)acetamido)propane-1,2-diyl diacetate (3)

To a mixture of (1R,2R)-1-((2R,3R,4S,6R)-4-acetoxy-3-(2-acetoxyacetamido)-6-(methoxycarbonyl)-6-tosyltetrahydro-2H-pyran-2-yl)-3-(2-(4-chlorophenyl)acetamido)propane-1,2-diyl diacetate (1, 0.40 g, 0.50 mmol), heptanal (2, 0.12 g, 1.0 mmol) was added freshly prepared samarium iodide solution (0.5 M in tetrahydrofuran, 30 mL) at room temperature and the reaction was stirred at room temperature for 10 min. After 10 min reaction mixture was quenched with saturated aqueous solution of sodium thiosulfate pentahydrate (10 mL), filtered, and the filtrate was extracted with dichloromethane, dried, filtered, and the filtrate was concentrated. The crude residue obtained was purified by silica gel column chromatography using ethyl acetate in heptane as eluents to afford the desired product (1R,2R)-1-((2R,3R,4S,6S)-4-acetoxy-3-(2-acetoxyacetamido)-6-((S)-1-hydroxyheptyl)-6-(methoxycarbonyl)tetrahydro-2H-pyran-2-yl)-3-(2-(4-chlorophenyl)acetamido)propane-1,2-diyl diacetate (3). Yield: 0.12 g, 31.58%; LCMS m/z 756.90 [M+H]⁺.

### Synthesis of (2S,4S,5R,6R)-6-((1R,2R)-3-(2-(4-chlorophenyl)acetamido)-1,2-dihydroxypropyl)-4-hydroxy-5-(2-hydroxyacetamido)-2-((S)-1-hydroxyheptyl)tetrahydro-2H-pyran-2-carboxylic acid (Cpd. 203)

To a stirred solution of (1R,2R)-1-((2R,3R,4S,6S)-4-acetoxy-3-(2-acetoxyacetamido)-6-((S)-1-hydroxyheptyl)-6-(methoxycarbonyl)tetrahydro-2H-pyran-2-yl)-3-(2-(4-chlorophenyl)acetamido)propane-1,2-diyl diacetate (3, 0.12 g, 0.16 mmol) in methanol (5.0 mL) was added a solution of lithium hydroxide monohydrate (0.023 g, 0.95 mmol) in water (0.5 mL). The reaction mixture was stirred at room temperature for 4 h. After completion, the reaction mixture was treated with acidic resin (Dowex 50H⁺) to pH ˜6 and the suspension was filtered. The filtrate was concentrated to get crude which was purified by prep HPLC using acetonitrile in water (+0.1% TFA) as eluents to afford the desired product (2S,4S,5R,6R)-6-((1R,2R)-3-(2-(4-chlorophenyl)acetamido)-1,2-dihydroxypropyl)-4-hydroxy-5-(2-hydroxyacetamido)-2-((S)-1-hydroxyheptyl)tetrahydro-2H-pyran-2-carboxylic acid (Cpd. 203). Yield: 0.039 g, 42%; LCMS m/z 575.30 [M+H]+; ¹H NMR (400 MHz, methanol-d₄): δ 8.07-8.02 (m, 0.5H), 7.89-7.86 (m, 0.5H), 7.32-7.27 (m, 4H), 4.01 (s, 2H), 3.88-3.59 (m, 5H), 3.52-3.50 (m, 3H), 3.37-3.32 (m, 1H), 3.23-3.17 (m, 1H), 2.53-2.43 (m, 1H), 2.09 (t, J=12.8 Hz, 0.5H), 1.77 (t, J=12.8 Hz, 0.5H), 1.57-1.41 (m, 2H), 1.40-1.20 (m, 8H), 0.92 (t, J=6.4 Hz, 3H).

### (2R,4S,5R,6R)-6-((1R,2R)-3-(2-(4-chlorophenyl)acetamido)-1,2-dihydroxypropyl)-2-heptyl-4-hydroxy-5-(2-hydroxyacetamido)tetrahydro-2H-pyran-2-carboxylic acid (Compound 206)

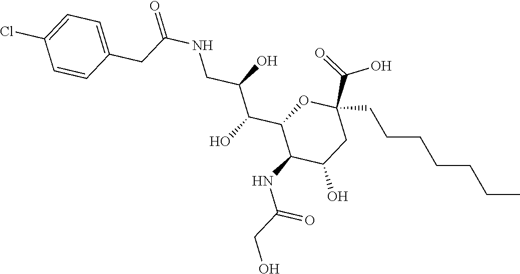 [SMILES: CCCCCCC[C@]1(C(=O)O)C[C@H](O)[C@@H](NC(=O)CO)[C@H]([C@H](O)[C@H](O)CNC(=O)Cc2ccc(Cl)cc2)O1]

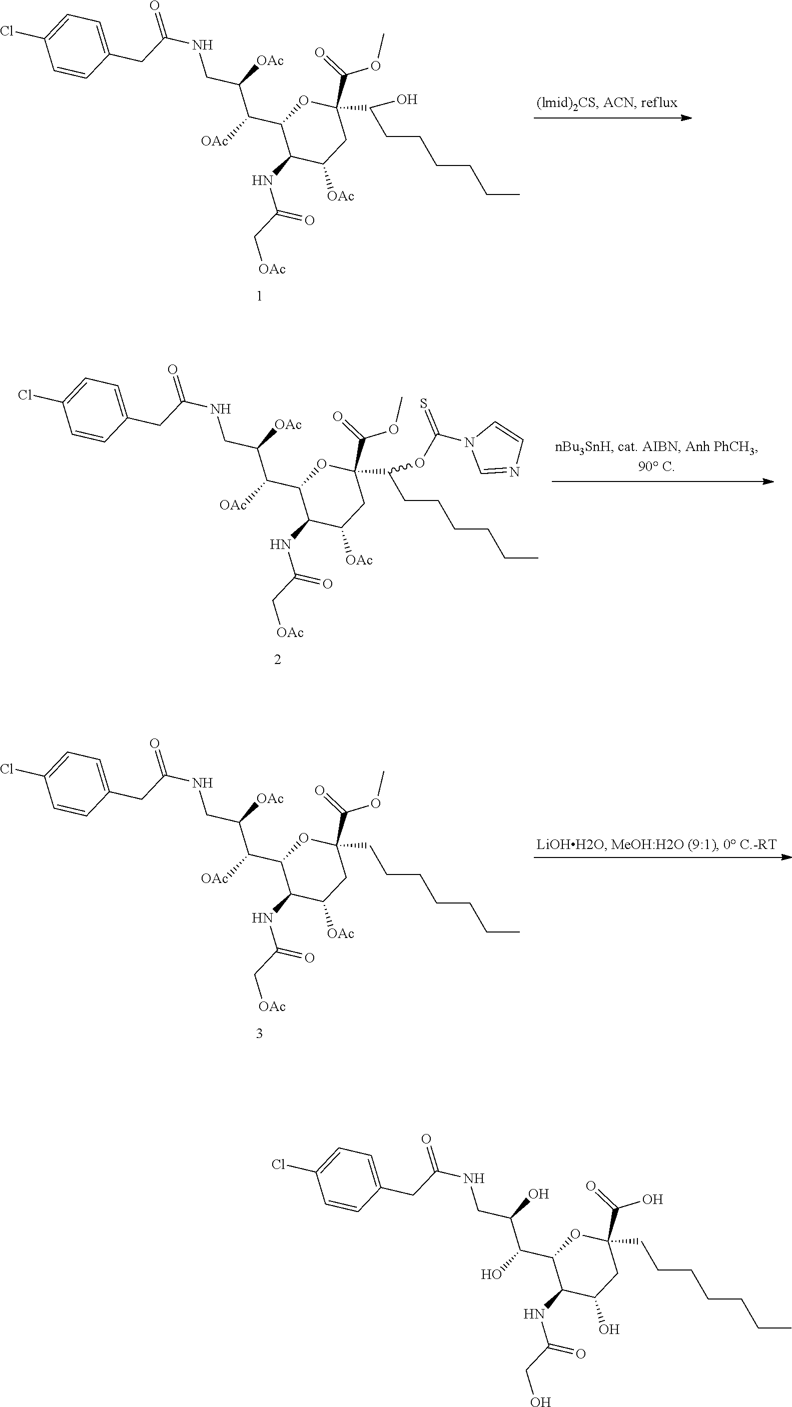 [SMILES: CCCCCCC(O)[C@]1(C(=O)OC)C[C@H](OC(C)=O)[C@@H](NC(=O)COC(C)=O)[C@H]([C@H](OC(C)=O)[C@@H](CNC(=O)Cc2ccc(Cl)cc2)OC(C)=O)O1 ; CCCCCCC(OC(=S)n1ccnc1)[C@]1(C(=O)OC)C[C@H](OC(C)=O)[C@@H](NC(=O)COC(C)=O)[C@H]([C@H](OC(C)=O)[C@@H](CNC(=O)Cc2ccc(Cl)cc2)OC(C)=O)O1 ; CCCCCCC[C@]1(C(=O)OC)C[C@H](OC(C)=O)[C@@H](NC(=O)COC(C)=O)[C@H]([C@H](OC(C)=O)[C@@H](CNC(=O)Cc2ccc(Cl)cc2)OC(C)=O)O1 ; CCCCCCC[C@]1(C(=O)O)C[C@H](O)[C@@H](NC(=O)CO)[C@H]([C@H](O)[C@H](O)CNC(=O)Cc2ccc(Cl)cc2)O1]

### Synthesis of (1R,2R)-1-((2R,3R,4S,6S)-6-(1-((1H-imidazole-1-carbonothioyl)oxy)heptyl)-4-acetoxy-3-(2-acetoxyacetamido)-6-(methoxycarbonyl)tetrahydro-2H-pyran-2-yl)-3-(2-(4-chlorophenyl)acetamido)propane-1,2-diyl diacetate (2)

A solution of (1R,2R)-1-((2R,3R,4S,6S)-4-acetoxy-3-(2-acetoxyacetamido)-6-((S)-1-hydroxyheptyl)-6-(methoxycarbonyl)tetrahydro-2H-pyran-2-yl)-3-(2-(4-chlorophenyl)acetamido)propane-1,2-diyl diacetate (1, 0.45 g, 0.59 mmol) and thiocarbonyl diimidazole (2.12 g, 11.90 mmol) in acetonitrile (15 mL) was refluxed overnight. The mixture was concentrated, and the residue obtained was purified by silica gel column chromatography using ethyl acetate in hexane to afford the desired product (1R,2R)-1-((2R,3R,4S,6S)-6-(1-((1H-imidazole-1-carbonothioyl)oxy)heptyl)-4-acetoxy-3-(2-acetoxyacetamido)-6-(methoxycarbonyl)tetrahydro-2H-pyran-2-yl)-3-(2-(4-chlorophenyl)acetamido)propane-1,2-diyl diacetate (2). This intermediate was immediately used for the next step. Yield: 0.30 g, 58.20%; LCMS m/z 867.00 [M+H]⁺.

### Synthesis of (1R,2R)-1-((2R,3R,4S,6R)-4-acetoxy-3-(2-acetoxyacetamido)-6-heptyl-6-(methoxycarbonyl)tetrahydro-2H-pyran-2-yl)-3-(2-(4-chlorophenyl)acetamido)propane-1,2-diyl diacetate (3)

To a stirred solution of (1R,2R)-1-((2R,3R,4S,6S)-6-(1-((1H-imidazole-1-carbonothioyl)oxy)heptyl)-4-acetoxy-3-(2-acetoxyacetamido)-6-(methoxycarbonyl)tetrahydro-2H-pyran-2-yl)-3-(2-(4-chlorophenyl)acetamido)propane-1,2-diyl diacetate (2, 0.35 g, 0.40 mmol) in anhydrous toluene (10 mL) under argon was added sequentially tributylstannane (0.29 g, 1.01 mmol) and azobisisobutyronitrile (catalytic). The solution was refluxed for 2 h and then concentrated. The residue was dissolved in acetonitrile and then washed twice with hexane. The acetonitrile layer was evaporated to dryness to obtain a crude which was purified by silica gel column chromatography using ethyl acetate in hexane to afford the desired product (1R,2R)-1-((2R,3R,4S,6R)-4-acetoxy-3-(2-acetoxyacetamido)-6-heptyl-6-(methoxycarbonyl)tetrahydro-2H-pyran-2-yl)-3-(2-(4-chlorophenyl)acetamido)propane-1,2-diyl diacetate (3). Yield: 0.07 g, 23.4%; LCMS m/z 741.05 [M+H]⁺.

### Synthesis of (2R,4S,5R,6R)-6-((1R,2R)-3-(2-(4-chlorophenyl)acetamido)-1,2-dihydroxypropyl)-2-heptyl-4-hydroxy-5-(2-hydroxyacetamido)tetrahydro-2H-pyran-2-carboxylic acid (Cpd. 206)

To a stirred solution of (1R,2R)-1-((2R,3R,4S,6R)-4-acetoxy-3-(2-acetoxyacetamido)-6-heptyl-6-(methoxycarbonyl)tetrahydro-2H-pyran-2-yl)-3-(2-(4-chlorophenyl)acetamido)propane-1,2-diyl diacetate (3, 0.07 g, 0.09 mmol) in methanol (5.0 mL) was added a solution of lithium hydroxide monohydrate (0.013 g, 0.57 mmol) in water (0.5 mL). The reaction mixture was stirred at room temperature for 10 h. After completion, the reaction mixture was treated with acidic resin (Dowex 50H⁺) to pH ˜6 and the suspension was filtered. The filtrate was concentrated to get crude which was purified by prep HPLC using acetonitrile in water (+0.1% TFA) as eluents to afford the desired product (2R,4S,5R,6R)-6-((1R,2R)-3-(2-(4-chlorophenyl)acetamido)-1,2-dihydroxypropyl)-2-heptyl-4-hydroxy-5-(2-hydroxyacetamido)tetrahydro-2H-pyran-2-carboxylic acid (Cpd. 206). Yield: 0.011 g, 20.42%; LCMS m/z 559.35 [M+H]+; ¹H NMR (400 MHz, methanol-d₄): δ 7.32-7.27 (m, 4H), 4.01 (s, 2H), 3.85 (td, J=8.0 Hz & 3.2 Hz, 1H), 3.83-3.76 (m, 1H), 3.73 (t, J=9.6 Hz, 1H), 3.67-3.62 (m, 2H), 3.53 (s, 2H), 3.35-3.33 (m, 1H), 3.25-3.19 (m, 1H), 2.62 (dd, J=13.2 Hz & 4.4 Hz, 1H), 1.75-1.72 (m, 2H), 1.56 (t, J=12.0 Hz, 1H), 1.48-1.46 (m, 1H), 1.34-1.49 (m, 9H), 0.90 (t, J=6.8 Hz, 1H).

### (2R,4S,5R,6R)-6-((1R,2R)-3-(2-(4-(difluoromethyl)phenyl)acetamido)-1,2-dihydroxypropyl)-2-((6-((3,4-dioxo-2-(prop-2-yn-1-ylamino)cyclobut-1-en-1-yl)amino)hexyl)oxy)-4-hydroxy-5-(2-hydroxyacetamido)tetrahydro-2H-pyran-2-carboxylic acid (Compound 220)

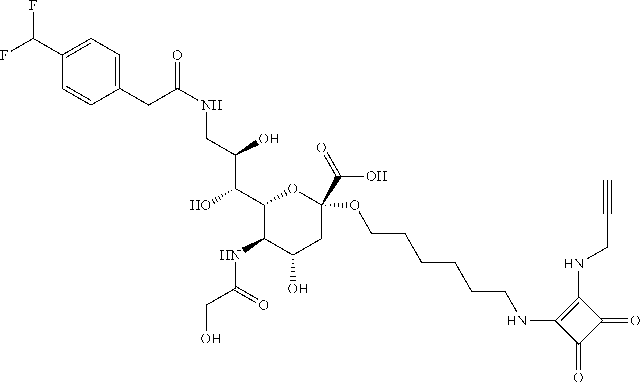 [SMILES: C#CCNc1c(NCCCCCCO[C@]2(C(=O)O)C[C@H](O)[C@@H](NC(=O)CO)[C@H]([C@H](O)[C@H](O)CNC(=O)Cc3ccc(C(F)F)cc3)O2)c(=O)c1=O]

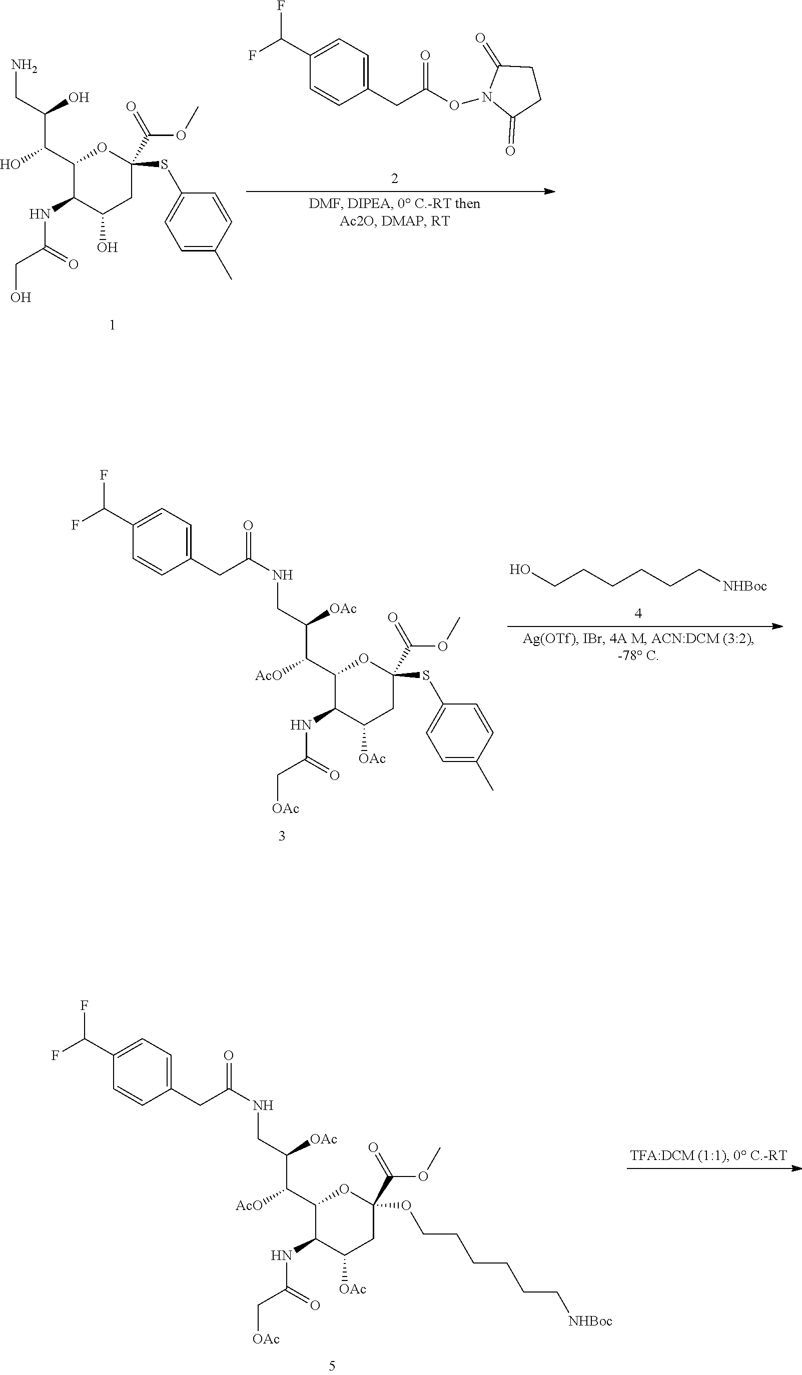 [SMILES: COC(=O)[C@]1(Sc2ccc(C)cc2)C[C@H](O)[C@@H](NC(=O)CO)[C@H]([C@H](O)[C@H](O)CN)O1 ; O=C(Cc1ccc(C(F)F)cc1)ON1C(=O)CCC1=O ; COC(=O)[C@]1(Sc2ccc(C)cc2)C[C@H](OC(C)=O)[C@@H](NC(=O)COC(C)=O)[C@H]([C@H](OC(C)=O)[C@@H](CNC(=O)Cc2ccc(C(F)F)cc2)OC(C)=O)O1 ; CC(C)(C)OC(=O)NCCCCCCO ; COC(=O)[C@@]1(OCCCCCCNC(=O)OC(C)(C)C)C[C@H](OC(C)=O)[C@@H](NC(=O)COC(C)=O)[C@H]([C@H](OC(C)=O)[C@@H](CNC(=O)Cc2ccc(C(F)F)cc2)OC(C)=O)O1]

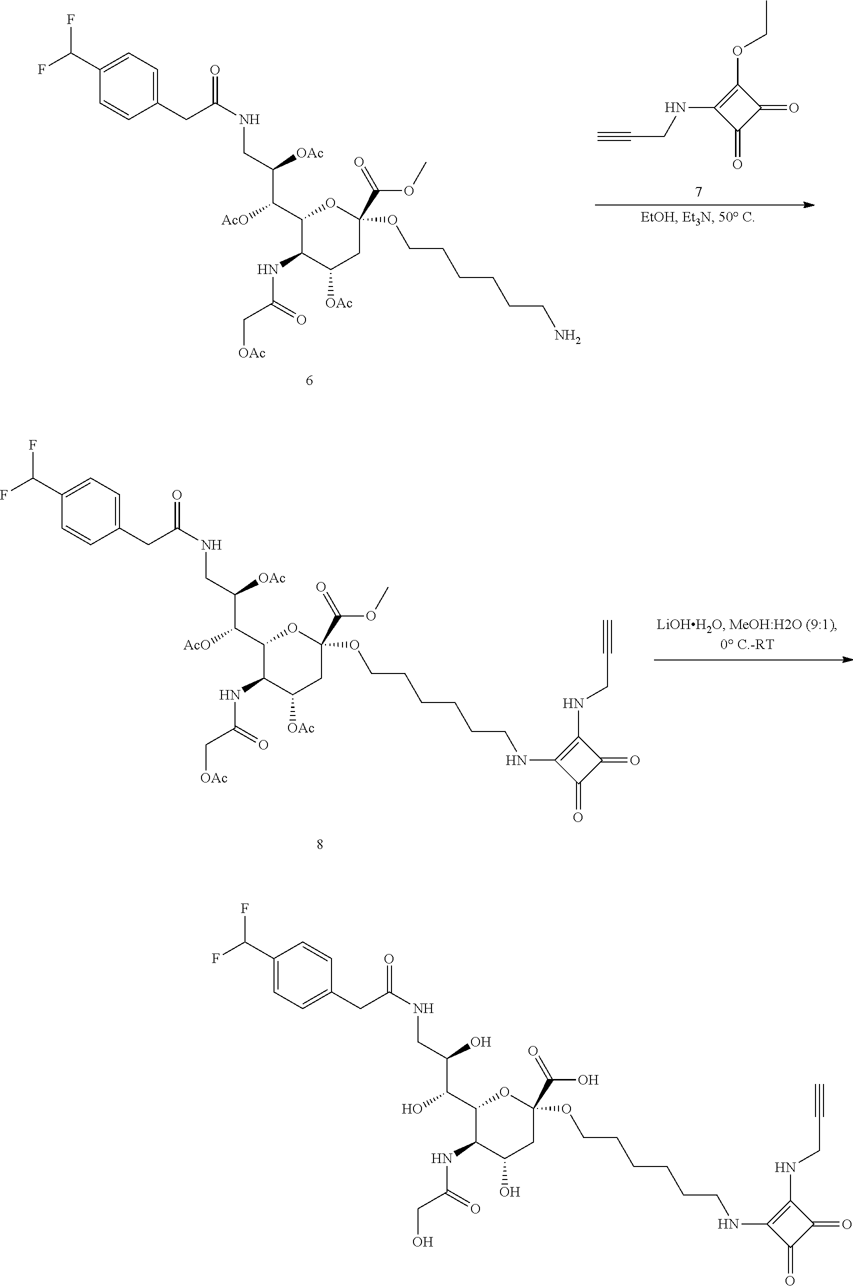 [SMILES: COC(=O)[C@@]1(OCCCCCCN)C[C@H](OC(C)=O)[C@@H](NC(=O)COC(C)=O)[C@H]([C@H](OC(C)=O)[C@@H](CNC(=O)Cc2ccc(C(F)F)cc2)OC(C)=O)O1 ; C#CCNc1c(OCC)c(=O)c1=O ; C#CCNc1c(NCCCCCCO[C@]2(C(=O)OC)C[C@H](OC(C)=O)[C@@H](NC(=O)COC(C)=O)[C@H]([C@H](OC(C)=O)[C@@H](CNC(=O)Cc3ccc(C(F)F)cc3)OC(C)=O)O2)c(=O)c1=O ; C#CCNc1c(NCCCCCCO[C@]2(C(=O)O)C[C@H](O)[C@@H](NC(=O)CO)[C@H]([C@H](O)[C@H](O)CNC(=O)Cc3ccc(C(F)F)cc3)O2)c(=O)c1=O]

### Synthesis of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-(difluoromethyl)phenyl)acetamido)propane-1,2-diyl diacetate (3)

To a stirred solution of methyl (2R,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (1, 1.57 g, 3.53 mmol) and 2,5-dioxopyrrolidin-1-yl 2-(4-(difluoromethyl)phenyl)acetate (2, 0.80 g, 2.83 mmol) in N, N-dimethylformamide (15.0 mL), was added ethylbis(propan-2-yl)amine (3.08 mL, 17.7 mmol) at 0° C. and the reaction mixture was stirred at room temperature for 4 h. Acetic anhydride (3.34 mL, 35.30 mmol) and 4-dimethylaminopyridine (0.43 g, 3.54 mmol) was added at 0° C. and stirred at room temperature for 12 h. After completion, the reaction mixture was diluted with water and ethyl acetate. The organic layer was separated, dried, filtered, and concentrated. The crude residue obtained was purified by silica gel column chromatography using ethyl acetate in hexane to afford the desired product (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-(difluoromethyl)phenyl)acetamido)propane-1,2-diyl diacetate (3). Yield: 1.20 g, 43.51% over two steps; LCMS (ESI) m/z 781.00 [M+H]⁺.

### Synthesis of (1R,2R)-1-((2R,3R,4S,6R)-4-acetoxy-3-(2-acetoxyacetamido)-6-((6-((tert-butoxycarbonyl)amino)hexyl)oxy)-6-(methoxycarbonyl)tetrahydro-2H-pyran-2-yl)-3-(2-(4-(difluoromethyl)phenyl)acetamido)propane-1,2-diyl diacetate (5)

A mixture of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-(difluoromethyl)phenyl)acetamido)propane-1,2-diyl diacetate (3, 0.40 g, 0.45 mmol) and tert-butyl (6-hydroxyhexyl)carbamate (4, 0.68 g, 3.12 mmol), silver(1+) trifluoromethanesulfonate (0.28 g, 1.11 mmol) and activated 4 Å powdered molecular sieves (1.00 g) in anhydrous dichloromethane (55.0 mL) and anhydrous acetonitrile (90.0 mL) was stirred at room temperature for 1 hr. The solution was cooled to −78° C. and iodobromane (0.14 g, 0.67 mmol) in dichloromethane (5.0 mL) was added dropwise to the reaction mixture and was stirred at same temperature for 2 h. After completion, the reaction mixture was quenched by triethylamine (1.0 mL) and warmed to room temperature. The reaction mixture was filtered. The filtrate was washed by saturated aqueous solution of sodium bicarbonate and dried, filtered, and concentrated. The crude residue obtained was purified by silica gel column chromatography using ethyl acetate in heptane to afford the desired product (1R,2R)-1-((2R,3R,4S,6R)-4-acetoxy-3-(2-acetoxyacetamido)-6-((6-((tert-butoxycarbonyl)amino)hexyl)oxy)-6-(methoxycarbonyl)tetrahydro-2H-pyran-2-yl)-3-(2-(4-(difluoromethyl)phenyl)acetamido)propane-1,2-diyl diacetate (5) as a white color solid. Yield: 0.30 g, 67%; LCMS m/z 874.35 [M+H]⁺.

### Synthesis of (1R,2R)-1-((2R,3R,4S,6R)-4-acetoxy-3-(2-acetoxyacetamido)-6-((6-aminohexyl)oxy)-6-(methoxycarbonyl)tetrahydro-2H-pyran-2-yl)-3-(2-(4--(difluoromethyl)acetamido)propane-1,2-diyl diacetate (6)

To a stirred solution of (1R,2R)-1-((2R,3R,4S,6R)-4-acetoxy-3-(2-acetoxyacetamido)-6-((6-((tert-butoxycarbonyl)amino)hexyl)oxy)-6-(methoxycarbonyl)tetrahydro-2H-pyran-2-yl)-3-(2-(4-(difluoromethyl)phenyl)acetamido)propane-1,2-diyl diacetate (5, 0.30 g, 0.34 mmol) in anhydrous dichloromethane (3.0 mL), trifluoroacetic acid (2.0 mL) was added at 0° C. The reaction mixture was stirred at room temperature for 3 h. After completion, solvent was concentrated to afford the desired product (1R,2R)-1-((2R,3R,4S,6R)-4-acetoxy-3-(2-acetoxyacetamido)-6-((6-aminohexyl)oxy)-6-(methoxycarbonyl)tetrahydro-2H-pyran-2-yl)-3-(2-(4--(difluoromethyl)acetamido)propane-1,2-diyl diacetate (6) as a light yellow sticky syrup. The crude residue was used for the next step without further purification. Yield: 0.20 g, Crude; LCMS (ESI) m/z 774.45 [M+H]⁺.

### Synthesis of (1R,2R)-1-((2R,3R,4S,6R)-4-acetoxy-3-(2-acetoxyacetamido)-6-((6-((3,4-dioxo-2-(prop-2-yn-1-ylamino)cyclobut-1-en-1-yl)amino)hexyl)oxy)-6-(methoxycarbonyl)tetrahydro-2H-pyran-2-yl)-3-(2-(4-(difluoromethyl)phenyl)acetamido)propane-1,2-diyl diacetate (8)

To a stirred solution of (1R,2R)-1-((2R,3R,4S,6R)-4-acetoxy-3-(2-acetoxyacetamido)-6-((6-aminohexyl)oxy)-6-(methoxycarbonyl)tetrahydro-2H-pyran-2-yl)-3-(2-(4--(difluoromethyl)acetamido)propane-1,2-diyl diacetate (6, 0.20 g, 0.26 mmol) in ethanol (5 mL), 3-ethoxy-4-[(prop-2-yn-1-yl)amino]cyclobut-3-ene-1,2-dione (7, 0.07 g, 0.39 mmol) was added at room temperature. After 10 min, triethylamine (0.18 mL, 1.30 mmol) was added dropwise at 0° C. The resulting reaction mixture was stirred at 50° C. for 3 h. After completion, the reaction mixture was concentrated to afford the desired product (1R,2R)-1-((2R,3R,4S,6R)-4-acetoxy-3-(2-acetoxyacetamido)-6-((6-((3,4-dioxo-2-(prop-2-yn-1-ylamino)cyclobut-1-en-1-yl)amino)hexyl)oxy)-6-(methoxycarbonyl)tetrahydro-2H-pyran-2-yl)-3-(2-(4-(difluoromethyl)phenyl)acetamido)propane-1,2-diyl diacetate (8) as a crude which was used for the next step without purification. Yield: 0.15 g, Crude; LCMS (ESI) m/z 906.60 [M+H]⁺.

### Synthesis of (2R,4S,5R,6R)-6-((1R,2R)-3-(2-(4-(difluoromethyl)phenyl)acetamido)-1,2-dihydroxypropyl)-2-((6-((3,4-dioxo-2-(prop-2-yn-1-ylamino)cyclobut-1-en-1-yl)amino)hexyl)oxy)-4-hydroxy-5-(2-hydroxyacetamido)tetrahydro-2H-pyran-2-carboxylic acid (Cpd. 220)

To a stirred solution of (1R,2R)-1-((2R,3R,4S,6R)-4-acetoxy-3-(2-acetoxyacetamido)-6-((6-((3,4-dioxo-2-(prop-2-yn-1-ylamino)cyclobut-1-en-1-yl)amino)hexyl)oxy)-6-(methoxycarbonyl)tetrahydro-2H-pyran-2-yl)-3-(2-(4-(difluoromethyl)phenyl)acetamido)propane-1,2-diyl diacetate (8, 0.15 g, 0.16 mmol) in methanol (10 mL) was added solution of lithium hydroxide monohydrate (0.023 g, 0.96 mmol) in water (0.1 mL). The reaction mixture was stirred at room temperature for 6 h. After completion, the reaction mixture was treated with acidic resin (Dowex 50, H⁺) to pH˜6 and the suspension was filtered. The filtrate was concentrated, and the crude residue obtained was purified by prep HPLC using acetonitrile in water (+0.1% TFA) as eluents to afford the desired product (2R,4S,5R,6R)-6-((1R,2R)-3-(2-(4-(difluoromethyl)phenyl)acetamido)-1,2-dihydroxypropyl)-2-((6-((3,4-dioxo-2-(prop-2-yn-1-ylamino)cyclobut-1-en-1-yl)amino)hexyl)oxy)-4-hydroxy-5-(2-hydroxyacetamido)tetrahydro-2H-pyran-2-carboxylic acid (Cpd. 220). Yield: 0.029 g, 24.19%; LCMS m/z 725.45 [M+H]⁺; ¹H NMR (400 MHz, Methanol-d₄): δ 7.49-7.41 (m, 4H), 6.73 (t, J=12.4 Hz, 1H), 4.42 (s, 2H), 4.02 (s, 2H), 3.88 (td, J=8.4 & 2.8 Hz, 1H), 3.84-3.68 (m, 5H), 3.61-3.56 (m, 4H), 3.43-3.37 (m, 2H), 3.24-3.18 (m, 1H), 2.82 (t, J=2.4 Hz, 1H), 2.70 (dd, J=12.4 Hz & 4.0 Hz, 1H), 1.72 (t, J=12.4 Hz, 1H), 1.59-1.54 (m, 4H), 1.40-1.35 (m, 4H).

### (2R,4S,5R,6R)-6-((1R,2R)-3-(2-([1,1′-biphenyl]-4-yl)acetamido)-1,2-dihydroxypropyl)-2-((6-((3,4-dioxo-2-(prop-2-yn-1-ylamino)cyclobut-1-en-1-yl)amino)hexyl)oxy)-4-hydroxy-5-(2-hydroxyacetamido)tetrahydro-2H-pyran-2-carboxylic acid (Compound 221)

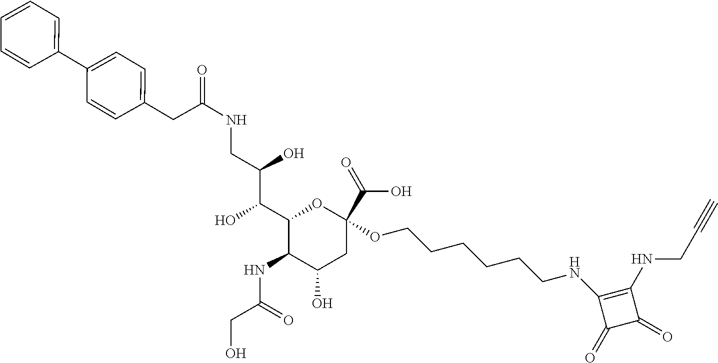 [SMILES: C#CCNc1c(NCCCCCCO[C@]2(C(=O)O)C[C@H](O)[C@@H](NC(=O)CO)[C@H]([C@H](O)[C@H](O)CNC(=O)Cc3ccc(-c4ccccc4)cc3)O2)c(=O)c1=O]

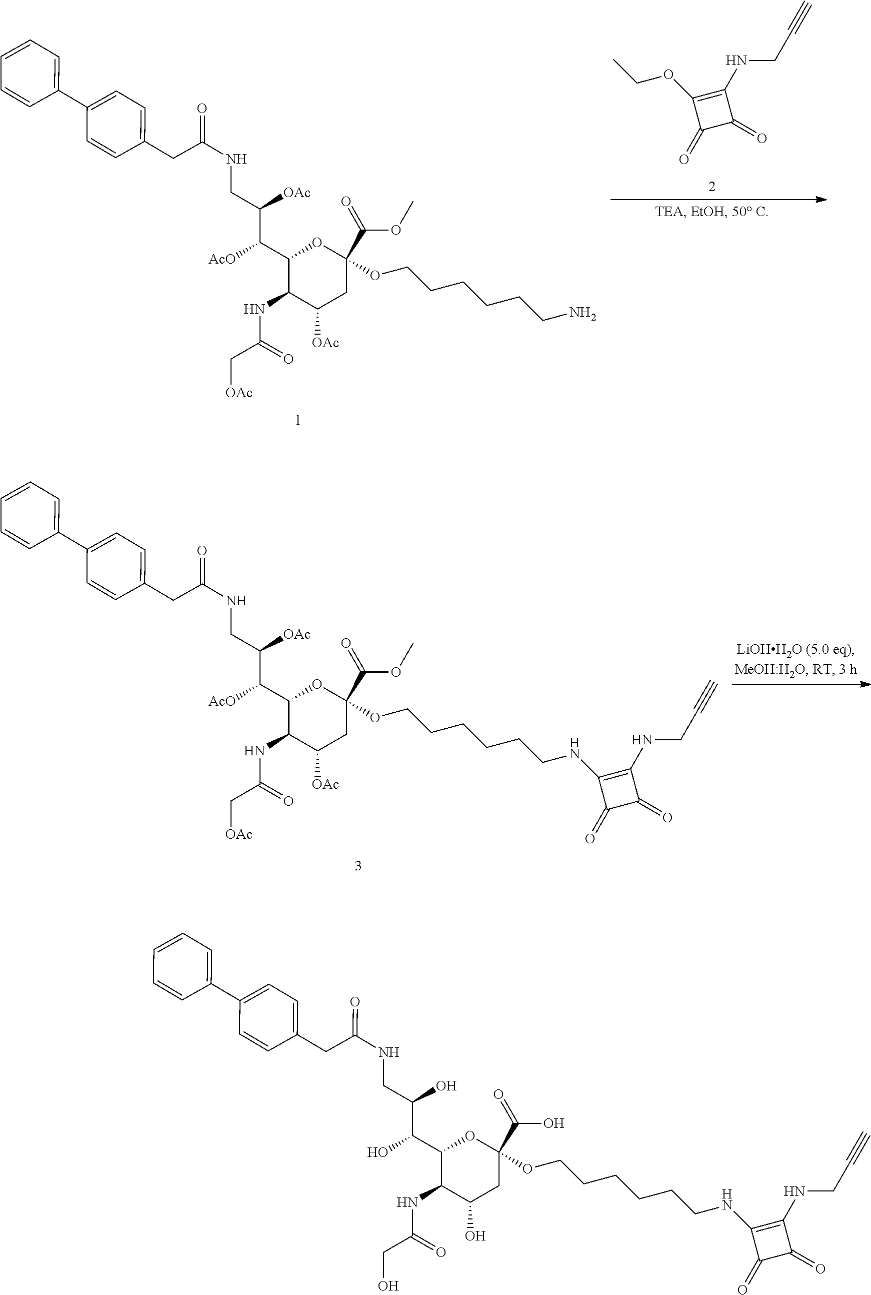 [SMILES: COC(=O)[C@@]1(OCCCCCCN)C[C@H](OC(C)=O)[C@@H](NC(=O)COC(C)=O)[C@H]([C@H](OC(C)=O)[C@@H](CNC(=O)Cc2ccc(-c3ccccc3)cc2)OC(C)=O)O1 ; C#CCNc1c(OCC)c(=O)c1=O ; C#CCNc1c(NCCCCCCO[C@]2(C(=O)OC)C[C@H](OC(C)=O)[C@@H](NC(=O)COC(C)=O)[C@H]([C@H](OC(C)=O)[C@@H](CNC(=O)Cc3ccc(-c4ccccc4)cc3)OC(C)=O)O2)c(=O)c1=O ; C#CCNc1c(NCCCCCCO[C@]2(C(=O)O)C[C@H](O)[C@@H](NC(=O)CO)[C@H]([C@H](O)[C@H](O)CNC(=O)Cc3ccc(-c4ccccc4)cc3)O2)c(=O)c1=O]

### Synthesis of (1R,2R)-3-(2-([1,1′-biphenyl]-4-yl)acetamido)-1-((2R,3R,4S,6R)-4-acetoxy-3-(2-acetoxyacetamido)-6-((6-((3,4-dioxo-2-(prop-2-yn-1-ylamino)cyclobut-1-en-1-yl)amino)hexyl)oxy)-6-(methoxycarbonyl)tetrahydro-2H-pyran-2-yl)propane-1,2-diyl diacetate (3)

A stirred solution of (1R,2R)-3-(2-([1,1′-biphenyl]-4-yl)acetamido)-1-((2R,3R,4S,6R)-4-acetoxy-3-(2-acetoxyacetamido)-6-((6-aminohexyl)oxy)-6-(methoxycarbonyl)tetrahydro-2H-pyran-2-yl)propane-1,2-diyl diacetate (1, 0.20 g, 0.250 mmol) in ethanol (5.0 mL) was cooled to 0° C. To this solution, triethylamine (0.105 mL, 0.75 mmol) and 3-ethoxy-4-(prop-2-yn-1-ylamino)cyclobut-3-ene-1,2-dione (0.067 g, 0.375 mmol) was added and reaction was maintained at 50° C. for 2 h. After completion, the reaction mixture was concentrated and the crude residue obtained was purified with trituration with diethyl ether that afforded (1R,2R)-3-(2-([1,1′-biphenyl]-4-yl)acetamido)-1-((2R,3R,4S,6R)-4-acetoxy-3-(2-acetoxyacetamido)-6-((6-((3,4-dioxo-2-(prop-2-yn-1-ylamino)cyclobut-1-en-1-yl)amino)hexyl)oxy)-6-(methoxycarbonyl)tetrahydro-2H-pyran-2-yl)propane-1,2-diyl diacetate (3) as yellow solid. Yield: 0.170 g, 73%; LCMS (ESI) m/z 933.65 [M+H]⁺.

### Synthesis of (2R,4S,5R,6R)-6-((1R,2R)-3-(2-([1,1′-biphenyl]-4-yl)acetamido)-1,2-dihydroxypropyl)-2-((6-((3,4-dioxo-2-(prop-2-yn-1-ylamino)cyclobut-1-en-1-yl)amino)hexyl)oxy)-4-hydroxy-5-(2-hydroxyacetamido)tetrahydro-2H-pyran-2-carboxylic acid (Cpd. 221)

To a stirred solution of (1R,2R)-3-(2-([1,1′-biphenyl]-4-yl)acetamido)-1-((2R,3R,4S,6R)-4-acetoxy-3-(2-acetoxyacetamido)-6-((6-((3,4-dioxo-2-(prop-2-yn-1-ylamino)cyclobut-1-en-1-yl)amino)hexyl)oxy)-6-(methoxycarbonyl)tetrahydro-2H-pyran-2-yl)propane-1,2-diyl diacetate (0.170 g, 0.182 mmol) in methanol (3.0 mL) was added lithium hydroxide (0.046 g, 1.092 mmol) solution in water (0.5 mL) at room temperature and the reaction was stirred at room temperature for 6 h. After completion, Dowex-hydrogen form was added up to pH 6 and the mixture was filtered. The filtrate was concentrated, and the residue obtained was purified with preparative HPLC acetonitrile in water (+0.1% TFA) to afford the desired product (2R,4S,5R,6R)-6-((1R,2R)-3-(2-([1,1′-biphenyl]-4-yl)acetamido)-1,2-dihydroxypropyl)-2-((6-((3,4-dioxo-2-(prop-2-yn-1-ylamino)cyclobut-1-en-1-yl)amino)hexyl)oxy)-4-hydroxy-5-(2-hydroxyacetamido)tetrahydro-2H-pyran-2-carboxylic acid (Cpd. 221). Yield: 0.039 g, 28%; LCMS (ESI) m/z 751.50 [M+H]+; 1H NMR (400 MHz, methanol-d4): δ 7.60-7.55 (m, 4H), 7.43-7.37 (m, 4H), 7.33-7.29 (m, 1H), 4.40 (s, 2H), 4.01 (s, 2H), 3.91-3.86 (m, 1H), 3.84-3.68 (m, 5H), 3.63-3.49 (m, 4H), 3.42-3.34 (m, 2H), 3.22-3.16 (m, 1H), 2.82 (t, J=2.4 Hz, 1H), 2.71 (dd, J=12.4 Hz & 3.6 Hz, 1H), 1.70 (t, J=12.0 Hz, 1H), 1.55-1.50 (m, 4H), 1.40-1.26 (m, 4H).

### (2R,4S,5R,6R)-6-((1R,2R)-3-(2-([1,1′-biphenyl]-4-yl)acetamido)-1,2-dihydroxypropyl)-2-((6-((2-(((1-(18-(2,5-dioxo-2,5-dihydro-1H-pyrrol-1-yl)-15-oxo-3,6,9,12-tetraoxa-16-azaoctadecyl)-1H-1,2,3-triazol-4-yl)methyl)amino)-3,4-dioxocyclobut-1-en-1-yl)amino)hexyl)oxy)-4-hydroxy-5-(2-hydroxyacetamido)tetrahydro-2H-pyran-2-carboxylic acid (Compound 221M)

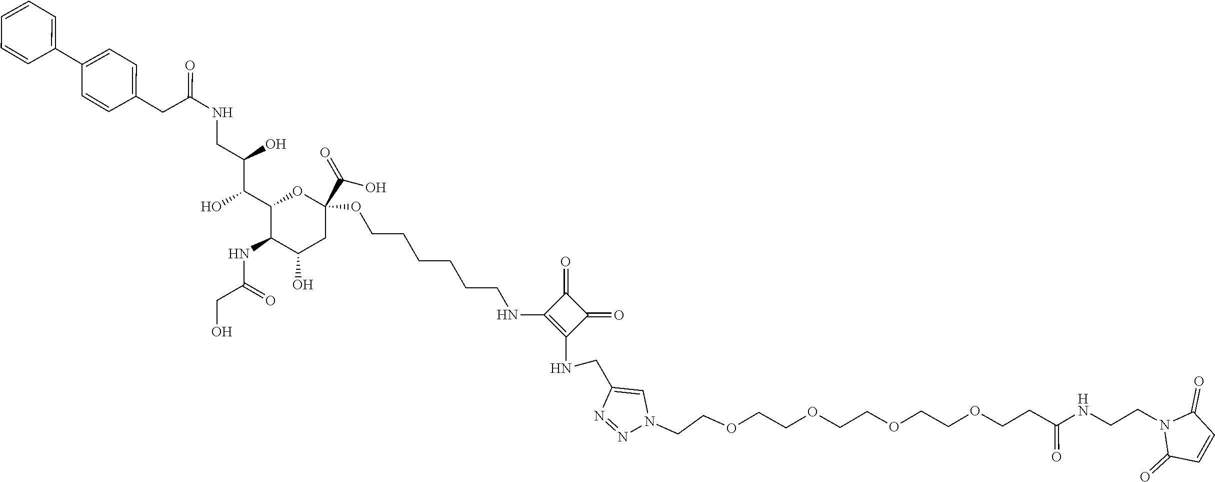

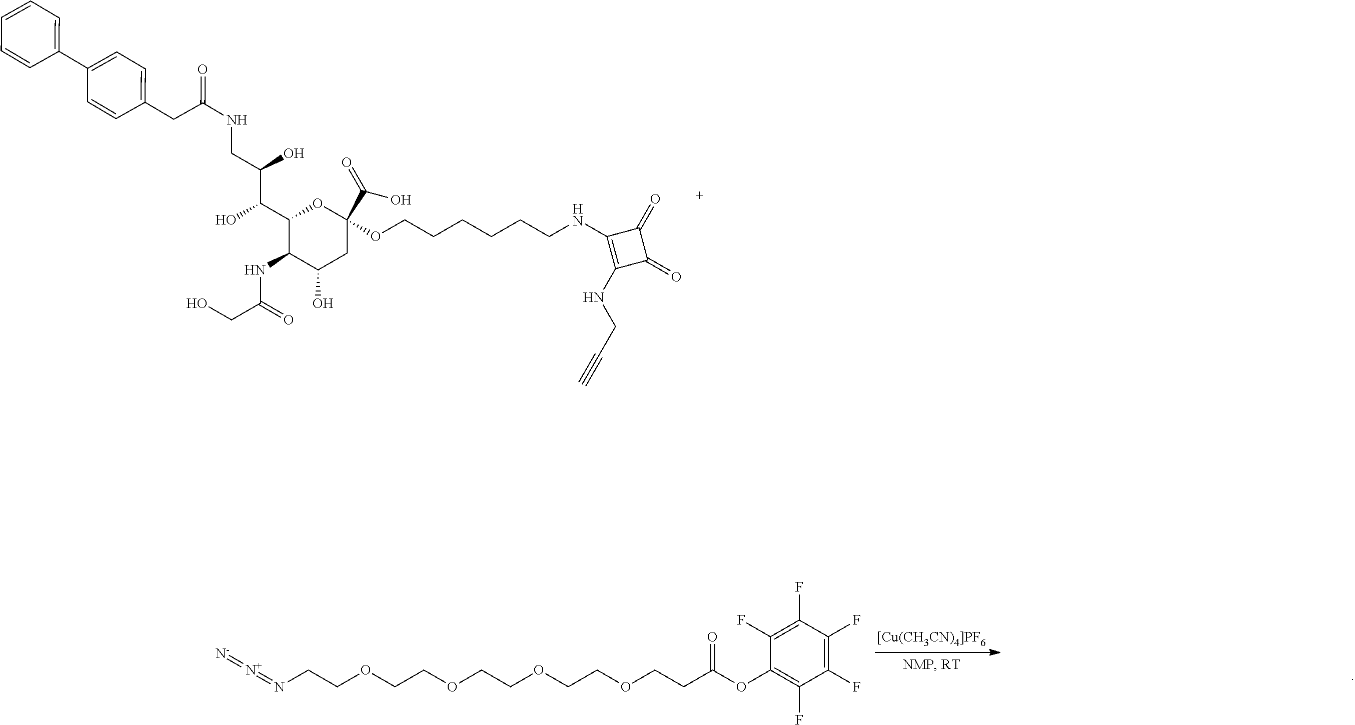

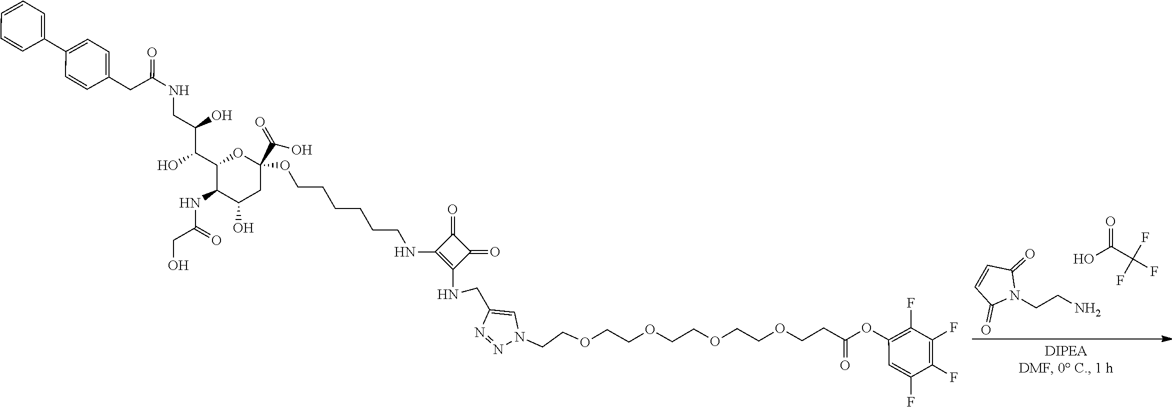

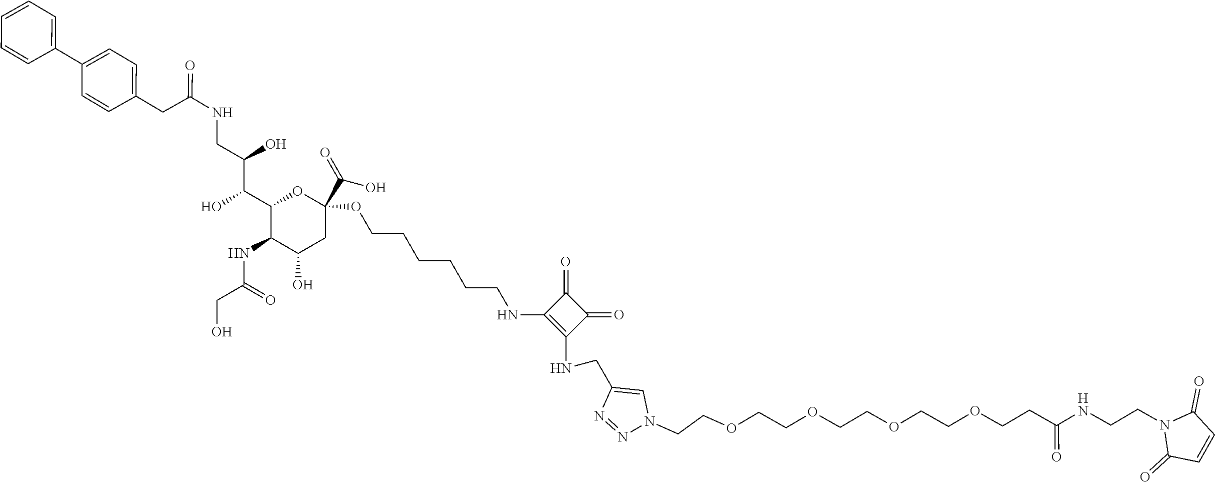

### Synthesis of (2R,4S,5R,6R)-6-((1R,2R)-3-(2-([1,1′-biphenyl]-4-yl)acetamido)-1,2-dihydroxypropyl)-2-((6-((3,4-dioxo-2-(((1-(15-oxo-15-(perfluorophenoxy)-3,6,9,12-tetraoxapentadecyl)-1H-1,2,3-triazol-4-yl)methyl)amino)cyclobut-1-en-1-yl)amino)hexyl)oxy)-4-hydroxy-5-(2-hydroxyacetamido)tetrahydro-2H-pyran-2-carboxylic acid (221P)

To a solution of (2R,4S,5R,6R)-6-((1R,2R)-3-(2-([1,1′-biphenyl]-4-yl)acetamido)-1,2-dihydroxypropyl)-2-((6-((3,4-dioxo-2-(prop-2-yn-1-ylamino)cyclobut-1-en-1-yl)amino)hexyl)oxy)-4-hydroxy-5-(2-hydroxyacetamido)tetrahydro-2H-pyran-2-carboxylic acid (221, 30.0 mg, 40.0 umol) in anhydrous NMP (258 uL) was added Azido-PEG4-PFP ester (21.9 mg, 47.9 umol) in anhydrous NMP (258 uL) dropwise, followed by Tetrakis(acetonitrile)copper(l) hexafluorophosphate (22.3 mg, 59.9 umol). The vial was sealed and the mixture was stirred for 45 min at room temperature. The mixture was filtered through a 0.2 μm PTFE filter, a few drops of acetic acid were added, and the filtrate was purified by C18 reverse phase column chromatography using acetonitrile and water (+0.1% TFA) as eluents. The fractions containing the desired product were lyophilized to afford 221P as a white solid. Yield: 32.4 mg, 67%; LCMS (ESI) m/z 1208 [M+H]⁺.

### Synthesis of (2R,4S,5R,6R)-6-((1R,2R)-3-(2-([1,1′-biphenyl]-4-yl)acetamido)-1,2-dihydroxypropyl)-2-((6-((2-(((1-(18-(2,5-dioxo-2,5-dihydro-1H-pyrrol-1-yl)-15-oxo-3,6,9,12-tetraoxa-16-azaoctadecyl)-1H-1,2,3-triazol-4-yl)methyl)amino)-3,4-dioxocyclobut-1-en-1-yl)amino)hexyl)oxy)-4-hydroxy-5-(2-hydroxyacetamido)tetrahydro-2H-pyran-2-carboxylic acid (221M)

To a solution of (2R,4S,5R,6R)-6-((1R,2R)-3-(2-([1,1′-biphenyl]-4-yl)acetamido)-1,2-dihydroxypropyl)-2-((6-((3,4-dioxo-2-(((1-(15-oxo-15-(perfluorophenoxy)-3,6,9,12-tetraoxapentadecyl)-1H-1,2,3-triazol-4-yl)methyl)amino)cyclobut-1-en-1-yl)amino)hexyl)oxy)-4-hydroxy-5-(2-hydroxyacetamido)tetrahydro-2H-pyran-2-carboxylic acid (221P, 32.0 mg, 26.5 umol) in DMF (638 uL) was added n-(2-aminoethyl)maleimide trifluoroacetate salt (8.08 mg, 31.8 umol). The mixture was cooled down to 0° C. and stirred for 10 min. DIEA (13.8 uL, 79.5 umol) was added and the mixture was stirred for 1 h at 0° C. The reaction mixture was filtered and purified by preparatory HPLC using acetonitrile and water (+0.05% TFA) as eluents. The fractions containing the desired product were combined and lyophilized to dryness to afford 221M as a white solid. Yield: 22.2 mg, 72%; LCMS (ESI) m/z 1164 [M+H]⁺. ¹H NMR (500 MHz, DMSO-d₆): 8.02-8.00 (m, 2H), 7.93 (t, J=5.5 Hz, 1H), 7.85 (d, J=8.0 Hz, 1H), 7.62 (d, J=8.0 Hz, 2H), 7.56 (d, J=8.0 Hz, 2H), 7.44 (t, J=8.0 Hz, 2H), 7.35-7.32 (m, 3H), 6.99 (s, 2H), 5.54 (s, br, 1H), 4.97 (s, br, 1H), 4.77-4.76 (m, 2H), 4.71 (s, br, 1H), 4.51 (t, J=5.5 Hz, 2H), 4.50 (s, 2H), 4.30 (s, br, 1H), 3.91-3.83 (m, 2H), 3.83-3.78 (m, 1H), 3.79 (t, J=5.5 Hz, 2H), 3.69-3.66 (m, 1H), 3.65-3.60 (m, 1H), 3.60-3.42 (m, 23H), 3.29-3.26 (m, 1H), 3.23-3.16 (m, 4H), 2.98-2.92 (m, 1H), 2.21 (t, J=6.5 Hz, 2H), 1.52 (t, J=12.0 Hz, 1H), 1.48-1.42 (m, 4H), 1.27-1.24 (m, 4H).

### (2R,4S,5R,6R)-6-((1R,2R)-3-(2-(4-chlorophenyl)acetamido)-1,2-dihydroxypropyl)-4-hydroxy-2-((4-(2-(1-(12-oxo-3,6,9-trioxa-13-azahexadecyl)-1H-1,2,3-triazol-4-yl)ethoxy)benzyl)oxy)-5-ureidotetrahydro-2H-pyran-2-carboxylic acid (Compound 222)

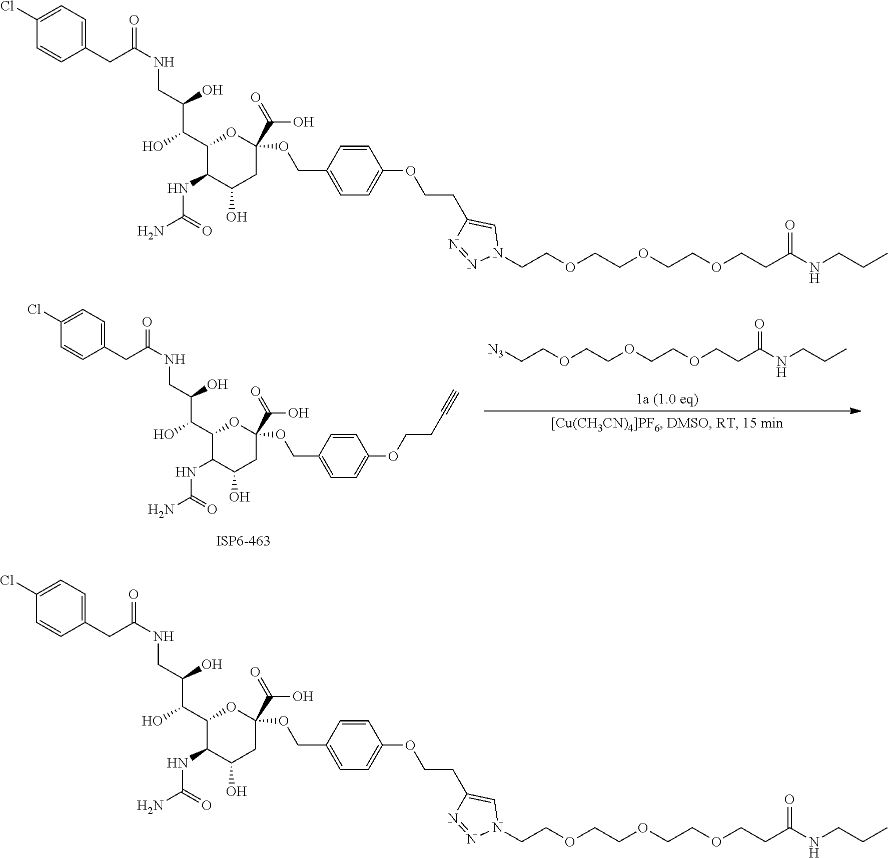

### Synthesis of (2R,4S,5R,6R)-6-((1R,2R)-3-(2-(4-chlorophenyl)acetamido)-1,2-dihydroxypropyl)-4-hydroxy-2-((4-(2-(1-(12-oxo-3,6,9-trioxa-13-azahexadecyl)-1H-1,2,3-triazol-4-yl)ethoxy)benzyl)oxy)-5-ureidotetrahydro-2H-pyran-2-carboxylic acid (Cpd. 222)

To a stirred solution of (2R,4S,5R,6R)-2-((4-(but-3-yn-1-yloxy)benzyl)oxy)-6-((1R,2R)-3-(2-(4-chlorophenyl)acetamido)-1,2-dihydroxypropyl)-4-hydroxy-5-ureidotetrahydro-2H-pyran-2-carboxylic acid (0.016 g, 0.025 mmol), 3-(2-(2-(2-azidoethoxy)ethoxy)ethoxy)-N-propylpropanamide (0.124 g, 0.025 mmol) in dimethyl sulfoxide (1 mL), was added λ¹-copper(1+) tetrakis(acetonitrile) hexafluoride λ⁻⁵-phosphanepentauide (0.026 g, 0.072 mmol) was stirred at room temperature for 15 min. After completion, acetic acid (0.1 mL) was added to the reaction mixture. The resulting solution was diluted with acetonitrile and purified by prep HPLC using acetonitrile in water (+0.1% TFA) to afford the desired product (2R,4S,5R,6R)-6-((1R,2R)-3-(2-(4-chlorophenyl)acetamido)-1,2-dihydroxypropyl)-4-hydroxy-2-((4-(2-(1-(12-oxo-3,6,9-trioxa-13-azahexadecyl)-1H-1,2,3-triazol-4-yl)ethoxy)benzyl)oxy)-5-ureidotetrahydro-2H-pyran-2-carboxylic acid (Cpd. 222). Yield: 0.013 g, 55.46%; LCMS m/z 908.65 [M+H]+; ¹H-NMR (400 MHz, Methanol-d₄): δ 7.97 (m, 1H), 7.24 (d, J=8.4 Hz, 2H), 6.89 (d, J=8.4 Hz, 2H), 4.72 (d, J=10.0 Hz, 1H), 4.55 (t, J=4.8 Hz, 2H), 4.40 (d, J=12.0 Hz, 1H), 4.24 (brs, 2H), 3.95-3.91 (m, 1H), 3.98-3.91 (m, 1H), 3.86 (t, J=9.6 Hz, 2H), 3.73-3.61 (m, 6H), 3.59-3.49 (m, 12H), 3.39-3.35 (m, 1H), 3.28-3.25 (m, 1H), 3.20-3.16 (m, 1H), 3.15-3.09 (m, 3H), 2.74 (d, J=10.8, 1H), 2.44-2.38 (m, 2H), 1.80-1.75 (m, 1H), 1.54-1.44 (m, 2H), 0.94-0.87 (m, 3H).

### (2R,4S,5R,6R)-6-((1R,2R)-3-(2-(4-chlorophenyl)acetamido)-1,2-dihydroxypropyl)-5-ethanethioamido-4-hydroxy-2-((6-(prop-2-yn-1-yloxy)hexyl)oxy)tetrahydro-2H-pyran-2-carboxylic acid (Compound 26)

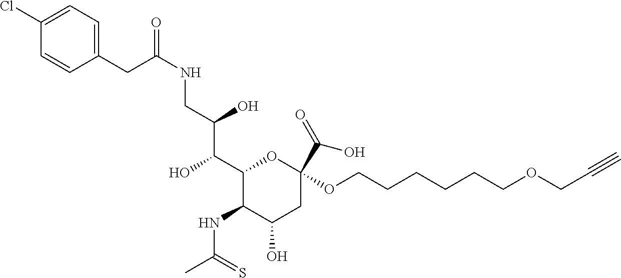

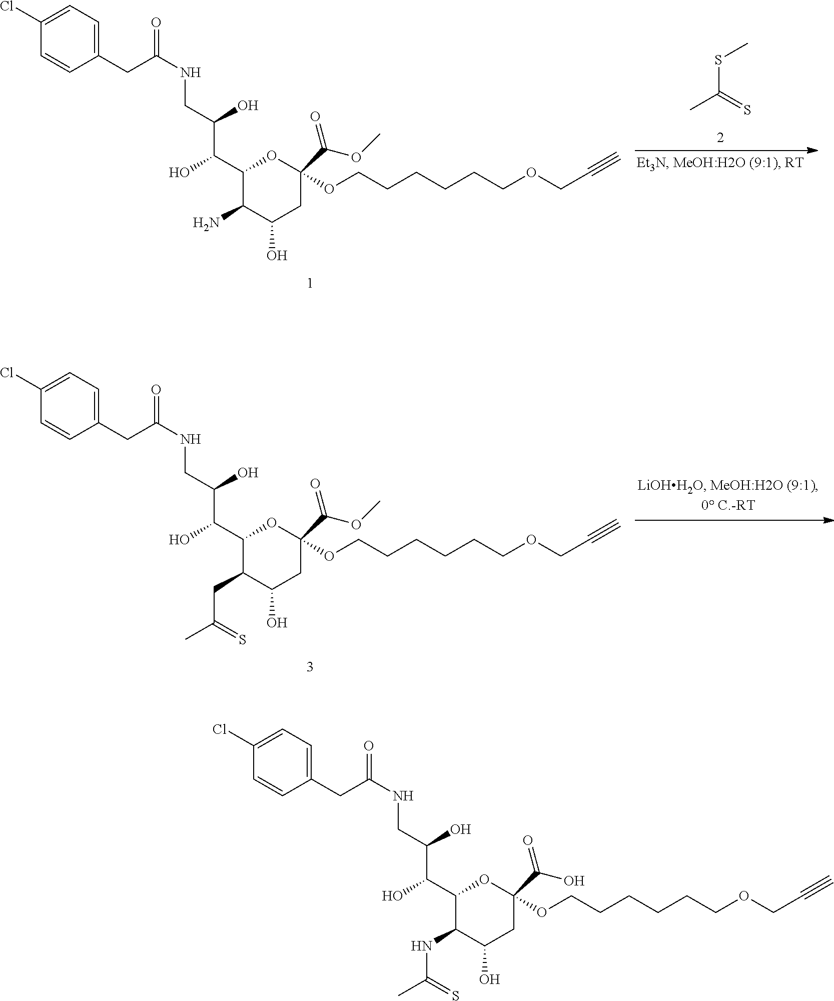

### Synthesis of methyl (2R,4S,5R,6R)-6-((1R,2R)-3-(2-(4-chlorophenyl)acetamido)-1,2-dihydroxypropyl)-5-ethanethioamido-4-hydroxy-2-((6-(prop-2-yn-1-yloxy)hexyl)oxy)tetrahydro-2H-pyran-2-carboxylate (3)

To a stirred solution of methyl (2R,4S,5R,6R)-5-amino-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylate (1, 0.15 g, 0.27 mmol) in a mixture of methanol and water (9:1; 10.0 mL) were sequentially added methyl ethanedithioate (0.28 g, 2.69 mmol) and triethylamine (0.38 mL, 2.69 mmol) dropwise at 0° C. and the reaction mixture was warmed up and stirred for 12 h at room temperature. After completion the reaction mixture was concentrated, and triturated using diethyl ether to afford the desired product (2R,4S,5R,6R)-6-((1R,2R)-3-(2-(4-chlorophenyl)acetamido)-1,2-dihydroxypropyl)-5-ethanethioamido-4-hydroxy-2-((6-(prop-2-yn-1-yloxy)hexyl)oxy)tetrahydro-2H-pyran-2-carboxylate (3) as a crude which was used for the next reaction without further purification; Yield: 0.11 g, 66%; LCMS m/z 628.90 [M+H]⁺.

### Synthesis of (2R,4S,5R,6R)-6-((1R,2R)-3-(2-(4-chlorophenyl)acetamido)-1,2-dihydroxypropyl)-5-ethanethioamido-4-hydroxy-2-((6-(prop-2-yn-1-yloxy)hexyl)oxy)tetrahydro-2H-pyran-2-carboxylic acid (Cpd. 26)

To a stirred solution of (2R,4S,5R,6R)-6-((1R,2R)-3-(2-(4-chlorophenyl)acetamido)-1,2-dihydroxypropyl)-5-ethanethioamido-4-hydroxy-2-((6-(prop-2-yn-1-yloxy)hexyl)oxy)tetrahydro-2H-pyran-2-carboxylate (3, 0.11 g, 0.17 mmol) in methanol (5.0 mL), was added a solution of lithium hydroxide monohydrate (0.025 g, 1.05 mmol) in water (1.0 mL). The reaction mixture was stirred at room temperature for 5 h. After completion, the reaction mixture was treated with acidic resin (Dowex 50, H⁺) to pH ˜6 and the suspension was filtered. The filtrate was concentrated. The crude residue obtained was purified by Prep-HPLC using acetonitrile in water (+0.1% TFA) as eluents to afford the desired product (2R,4S,5R,6R)-6-((1R,2R)-3-(2-(4-chlorophenyl)acetamido)-1,2-dihydroxypropyl)-5-ethanethioamido-4-hydroxy-2-((6-(prop-2-yn-1-yloxy)hexyl)oxy)tetrahydro-2H-pyran-2-carboxylic acid (Cpd. 26). Yield: 0.019 g, 18%; LCMS m/z 615.30 [M+H]+; ¹H-NMR (400 MHz, Methanol-d₄): δ 7.32-7.17 (m, 4H), 4.47 (t, J=10.0 Hz, 1H), 4.12 (d, J=2.4 Hz, 1H), 3.91-3.81 (m, 2H), 3.80-3.71 (m, 2H), 3.60 (dd, J=14.0 Hz & 3.2 Hz, 1H), 3.55-3.51 (m, 4H), 3.44-3.40 (m, 1H), 3.38-3.32 (m, 2H), 2.81 (t, J=2.4 Hz, 1H), 2.70 (dd, J=12.8 Hz & 4.8 Hz, 1H), 2.50 (s, 3H), 1.76 (t, J=12.0 Hz, 1H), 1.57-1.55 (m, 4H), 1.39-1.36 (m, 4H).

### (2R,4S,5R,6R)-5-(2-(2H-tetrazol-5-yl)acetamido)-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (Compound 208)

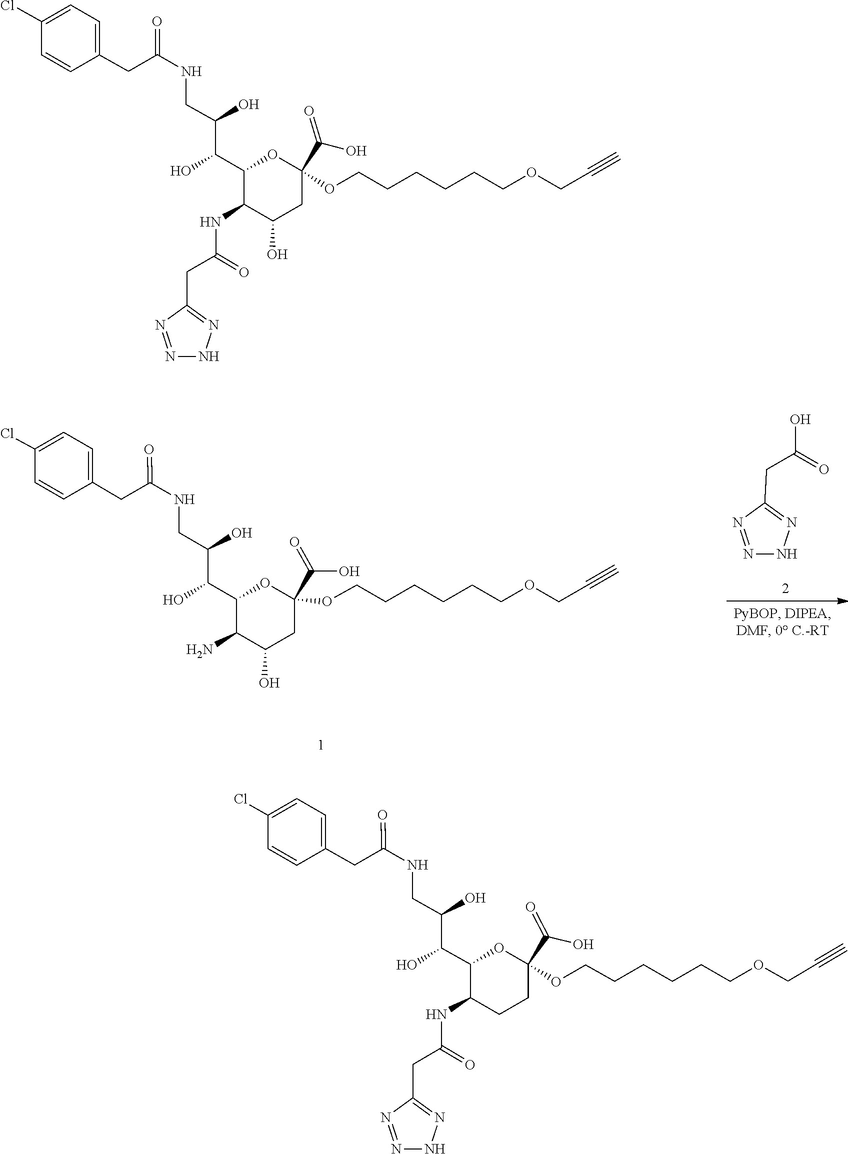 [SMILES: C#CCOCCCCCCO[C@]1(C(=O)O)C[C@H](O)[C@@H](NC(=O)Cc2nn[nH]n2)[C@H]([C@H](O)[C@H](O)CNC(=O)Cc2ccc(Cl)cc2)O1 ; C#CCOCCCCCCO[C@]1(C(=O)O)C[C@H](O)[C@@H](N)[C@H]([C@H](O)[C@H](O)CNC(=O)Cc2ccc(Cl)cc2)O1 ; O=C(O)Cc1nn[nH]n1 ; C#CCOCCCCCCO[C@]1(C(=O)O)CC[C@@H](NC(=O)Cc2nn[nH]n2)[C@H]([C@H](O)[C@H](O)CNC(=O)Cc2ccc(Cl)cc2)O1]

### Synthesis of (2R,4S,5R,6R)-5-(2-(2H-tetrazol-5-yl)acetamido)-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (Cpd. 208)

To a stirred solution of (2R,4S,5R,6R)-5-amino-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (1, 0.07 g, 0.126 mmol) and 2-(2H-tetrazol-5-yl)acetic acid (2, 0.024 g, 0.18 mmol) in N,N-dimethylformamide (2.0 mL) was added benzotriazol-1-yloxytripyrrolidinophosphonium hexafluorophosphate (0.13 g, 0.25 mmol) at 0° C. After 10 min ethylbis(propan-2-yl)amine (0.06 mL, 0.38 mmol) was added dropwise and the reaction mixture was stirred at room temperature for 24 h. After completion the reaction mixture was concentrated and the crude residue obtained was purified by Prep-HPLC using acetonitrile in water (+0.1% TFA) as eluents to afford the desired product (2R,4S,5R,6R)-5-(2-(2H-tetrazol-5-yl)acetamido)-6-((1R,2R)-3-(2-(4-chlorophenyl)acetamido)-1,2-dihydroxypropyl)-4-hydroxy-2-((6-(prop-2-yn-1-yloxy)hexyl)oxy)tetrahydro-2H-pyran-2-carboxylic acid (Cpd. 208). Yield: 0.034 g, 40%; LCMS m/z 667.3 [M+H]+; ¹H-NMR (400 MHz, Methanol-d₄): δ 7.30-7.28 (m, 4H), 4.13 (d, J=11.2 Hz, 1H), 4.05-3.98 (m, 1H), 3.94-3.84 (m, 2H), 3.81-3.78 (m, 1H), 3.76-3.71 (m, 2H), 3.67 (dd, J=10.4 Hz & 1.6 Hz, 1H), 3.60 (dd, J=13.6 Hz & 3.2 Hz, 1H), 3.53-3.49 (m, 4H), 3.42-3.39 (m, 2H), 3.29-3.27 (m, 1H), 2.80 (t, J=2.4 Hz, 1H), 2.71 (dd, J=12.8 Hz & 4.4 Hz, 1H), 1.72 (t, J=12.0 Hz, 1H), 1.57-1.55 (m, 4H), 1.38-1.36 (m, 4H).

### (2R,4S,5R,6R)-2-((6-benzamidohexyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Compound 108)

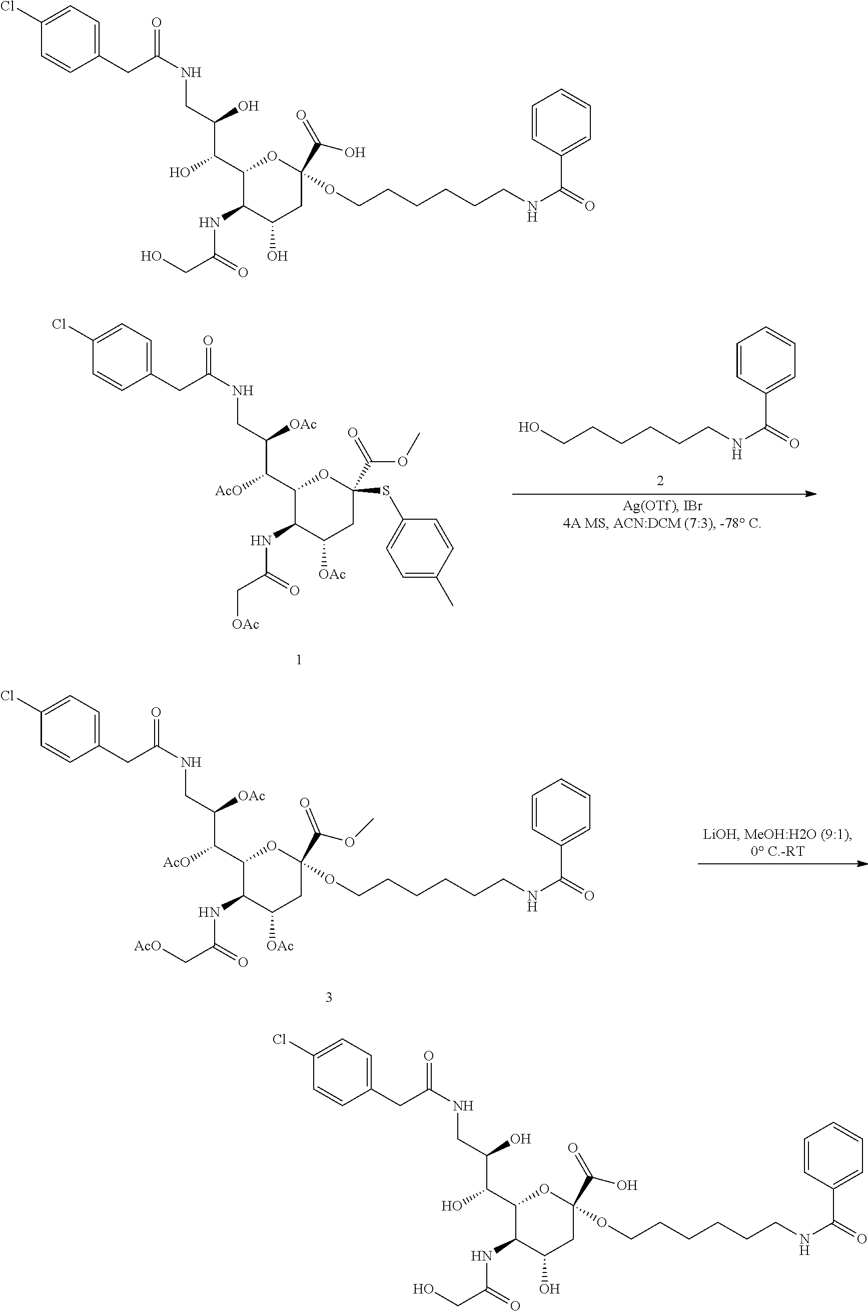 [SMILES: O=C(Cc1ccc(Cl)cc1)NC[C@@H](O)[C@@H](O)[C@@H]1O[C@@](OCCCCCCNC(=O)c2ccccc2)(C(=O)O)C[C@H](O)[C@H]1NC(=O)CO ; COC(=O)[C@]1(Sc2ccc(C)cc2)C[C@H](OC(C)=O)[C@@H](NC(=O)COC(C)=O)[C@H]([C@H](OC(C)=O)[C@@H](CNC(=O)Cc2ccc(Cl)cc2)OC(C)=O)O1 ; O=C(NCCCCCCO)c1ccccc1 ; COC(=O)[C@@]1(OCCCCCCNC(=O)c2ccccc2)C[C@H](OC(C)=O)[C@@H](NC(=O)COC(C)=O)[C@H]([C@H](OC(C)=O)[C@@H](CNC(=O)Cc2ccc(Cl)cc2)OC(C)=O)O1 ; O=C(Cc1ccc(Cl)cc1)NC[C@@H](O)[C@@H](O)[C@@H]1O[C@@](OCCCCCCNC(=O)c2ccccc2)(C(=O)O)C[C@H](O)[C@H]1NC(=O)CO]

### Synthesis of (1R,2R)-1-((2R,3R,4S,6R)-4-acetoxy-3-(2-acetoxyacetamido)-6-((6-benzamidohexyl)oxy)-6-(methoxycarbonyl)tetrahydro-2H-pyran-2-yl)-3-(2-(4-chlorophenyl)acetamido)propane-1,2-diyl diacetate (3)

A solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (1, 0.30 g, 0.39 mmol) and N-(6-hydroxyhexyl)benzamide (2, 0.52 g, 2.35 mmol), silver(1+) trifluoromethanesulfonate (0.30 g, 1.18 mmol) and activated 4 Å powdered molecular sieves (1.00 g) in anhydrous dichloromethane (55 mL) and anhydrous acetonitrile (90 mL) was stirred at room temperature for 1 h. The solution was cooled to −78° C. and iodobromane (0.16 g, 0.78 mmol) solution in dichloromethane (5.0 mL) was added dropwise to the reaction mixture and was stirred at same temperature for 2 h. After completion, the reaction mixture was quenched by addition of triethylamine (2.0 mL) and warmed up to room temperature. The reaction mixture was filtered. The filtrate was washed with saturated solution of sodium bicarbonate, dried, filtered and concentrated. The crude product was purified by silica gel column chromatography using ethyl acetate in heptane to afford the desired product (1R,2R)-1-((2R,3R,4S,6R)-4-acetoxy-3-(2-acetoxyacetamido)-6-((6-benzamidohexyl)oxy)-6-(methoxycarbonyl)tetrahydro-2H-pyran-2-yl)-3-(2-(4-chlorophenyl)acetamido)propane-1,2-diyl diacetate (3). Yield: 0.15 g, 44%; LCMS m/z 862.15 [M+H]⁺.

### Synthesis of (2R,4S,5R,6R)-2-((6-benzamidohexyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Cpd. 108)

To a stirred solution of (1R,2R)-1-((2R,3R,4S,6R)-4-acetoxy-3-(2-acetoxyacetamido)-6-((6-benzamidohexyl)oxy)-6-(methoxycarbonyl)tetrahydro-2H-pyran-2-yl)-3-(2-(4-chlorophenyl)acetamido)propane-1,2-diyl diacetate (3, 0.35 g, 0.42 mmol) in methanol (5 mL), was added a solution of lithium hydroxide monohydrate (0.058 g, 2.44 mmol) in water (0.5 mL). The reaction mixture was stirred at room temperature for 6 h. After completion, the reaction mixture was treated with acidic resin (Dowex 50, H⁺) to pH ˜6 and the suspension was filtered. The filtrate was concentrated. The crude residue obtained was purified by prep HPLC using acetonitrile in water (+0.1% TFA) to afford the desired product (2R,4S,5R,6R)-2-((6-benzamidohexyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (Cpd. 108). Yield: 0.057 g, 43%; LCMS m/z 680.30 [M+H]⁺; ¹H NMR (400 MHz, Methanol-d₄) δ 7.92 (d. 1H), 7.83-7.81 (m, 2H), 7.56-7.45 (m, 3H), 7.32-7.27 (m, 4H), 4.04 (s, 2H), 3.93-3.76 (m, 4H), 3.73-3.65 (m, 2H), 3.44-3.37 (m, 4H), 3.27-3.22 (m, 1H), 2.73 (dd, J=12.4 and 3.6 Hz, 1H), 1.75 (t, J=12.4 Hz, 1H), 1.65-1.57 (m, 4H), 1.46-1.42 (m, 4 Hz).

### (2S,4S,5R,6R)-2-((4-(but-3-yn-1-yloxy)benzyl)thio)-6-((1R,2R)-3-(2-(4-chloro-3-hydroxyphenyl)acetamido)-1,2-dihydroxypropyl)-4-hydroxy-5-(2-hydroxyacetamido)tetrahydro-2H-pyran-2-carboxylic acid (Compound 131)

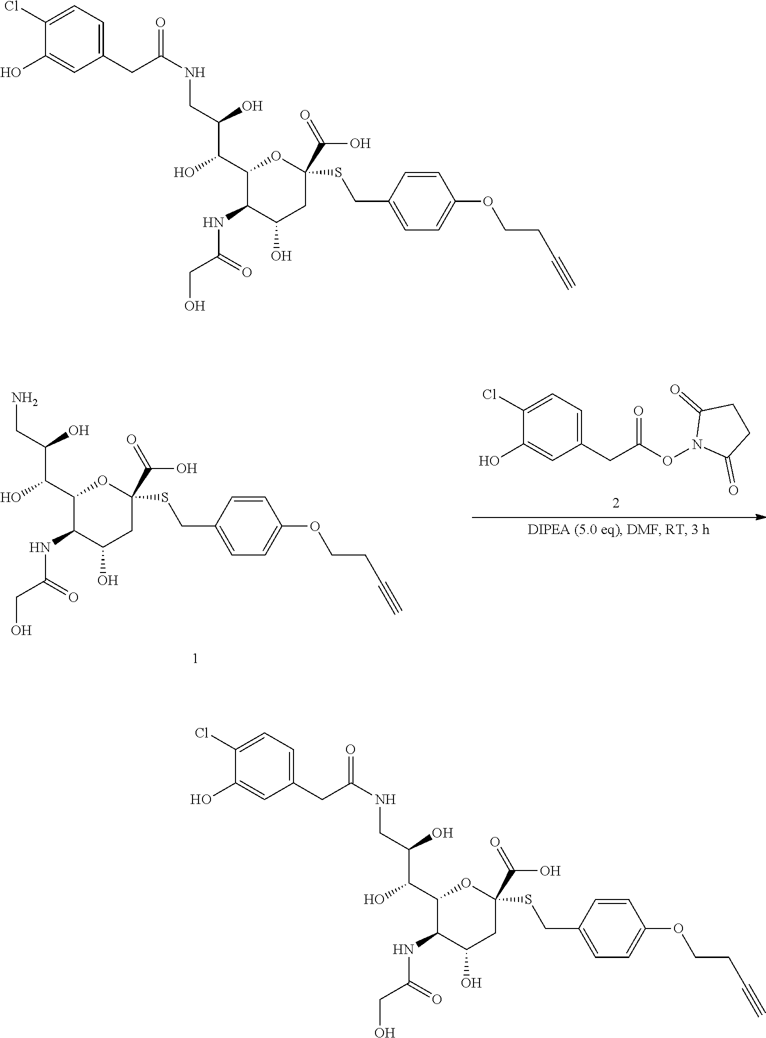 [SMILES: C#CCCOc1ccc(CS[C@]2(C(=O)O)C[C@H](O)[C@@H](NC(=O)CO)[C@H]([C@H](O)[C@H](O)CNC(=O)Cc3ccc(Cl)c(O)c3)O2)cc1 ; C#CCCOc1ccc(CS[C@]2(C(=O)O)C[C@H](O)[C@@H](NC(=O)CO)[C@H]([C@H](O)[C@H](O)CN)O2)cc1 ; O=C(Cc1ccc(Cl)c(O)c1)ON1C(=O)CCC1=O ; C#CCCOc1ccc(CS[C@]2(C(=O)O)C[C@H](O)[C@@H](NC(=O)CO)[C@H]([C@H](O)[C@H](O)CNC(=O)Cc3ccc(Cl)c(O)c3)O2)cc1]

### Synthesis of (2S,4S,5R,6R)-2-((4-(but-3-yn-1-yloxy)benzyl)thio)-6-((1R,2R)-3-(2-(4-chloro-3-hydroxyphenyl)acetamido)-1,2-dihydroxypropyl)-4-hydroxy-5-(2-hydroxyacetamido)tetrahydro-2H-pyran-2-carboxylic acid (Cpd. 131)

To a stirred solution of (2S,4S,5R,6R)-6-((1R,2R)-3-amino-1,2-dihydroxypropyl)-2-((4-(but-3-yn-1-yloxy)benzyl)thio)-4-hydroxy-5-(2-hydroxyacetamido)tetrahydro-2H-pyran-2-carboxylic acid (1, 0.300 g, 0.601 mmol) and 2,5-dioxopyrrolidin-1-yl 2-(4-chloro-3-hydroxyphenyl)acetate (2, 0.136 g, 0.481 mmol) in N,N-dimethylformamide (3.0 mL) was added ethylbis(propan-2-yl)amine (0.52 mL, 3.00 mmol) at 0° C. and the reaction mixture was stirred at room temperature for 4 h. After completion the reaction mixture was concentrated. The crude residue obtained was purified by prep HPLC using acetonitrile in water (+0.1% TFA) to afford the desired product (2S,4S,5R,6R)-2-((4-(but-3-yn-1-yloxy)benzyl)thio)-6-((1R,2R)-3-(2-(4-chloro-3-hydroxyphenyl)acetamido)-1,2-dihydroxypropyl)-4-hydroxy-5-(2-hydroxyacetamido)tetrahydro-2H-pyran-2-carboxylic acid (Cpd. 131). Yield: 0.053 g; 13%; LCMS (ESI) m/z 667.25 [M+H]+;¹H NMR (400 MHz, MeOD-d₄): δ 7.22 (d, J=8.4 Hz, 2H), 7.13 (d, J=8.4 Hz, 1H), 6.86-6.83 (m, 3H), 6.71 (dd, J=8.0 Hz & 2.0 Hz, 1H), 4.05 (t, J=6.8 Hz, 2H), 4.02 (s, 2H), 3.98 (d, J=12.4 Hz, 1H), 3.91-3.78 (m, 4H), 3.66-3.61 (m, 2H), 3.44 (s, 2H), 3.37 (dd, J=8.8 Hz & 1.6 Hz, 1H), 3.26-3.20 (m, 1H), 2.78 (dd, J=12.8 Hz & 4.4 Hz, 1H), 2.63 (td, J=6.4 Hz & 2.4 Hz, 2H), 2.31 (t, J=2.8 Hz, 1H), 1.81-1.75 (m, 1H).

### (2R,4S,5R,6R)-6-((1R,2R)-1,2-dihydroxy-3-(2-(4′-hydroxy-[1,1′-biphenyl]-4-yl)acetamido)propyl)-4-hydroxy-5-((N-methylsulfamoyl)amino)-2-(2-(2-(prop-2-yn-1-yloxy)ethoxy)ethoxy)tetrahydro-2H-pyran-2-carboxylic acid (Compound 225)

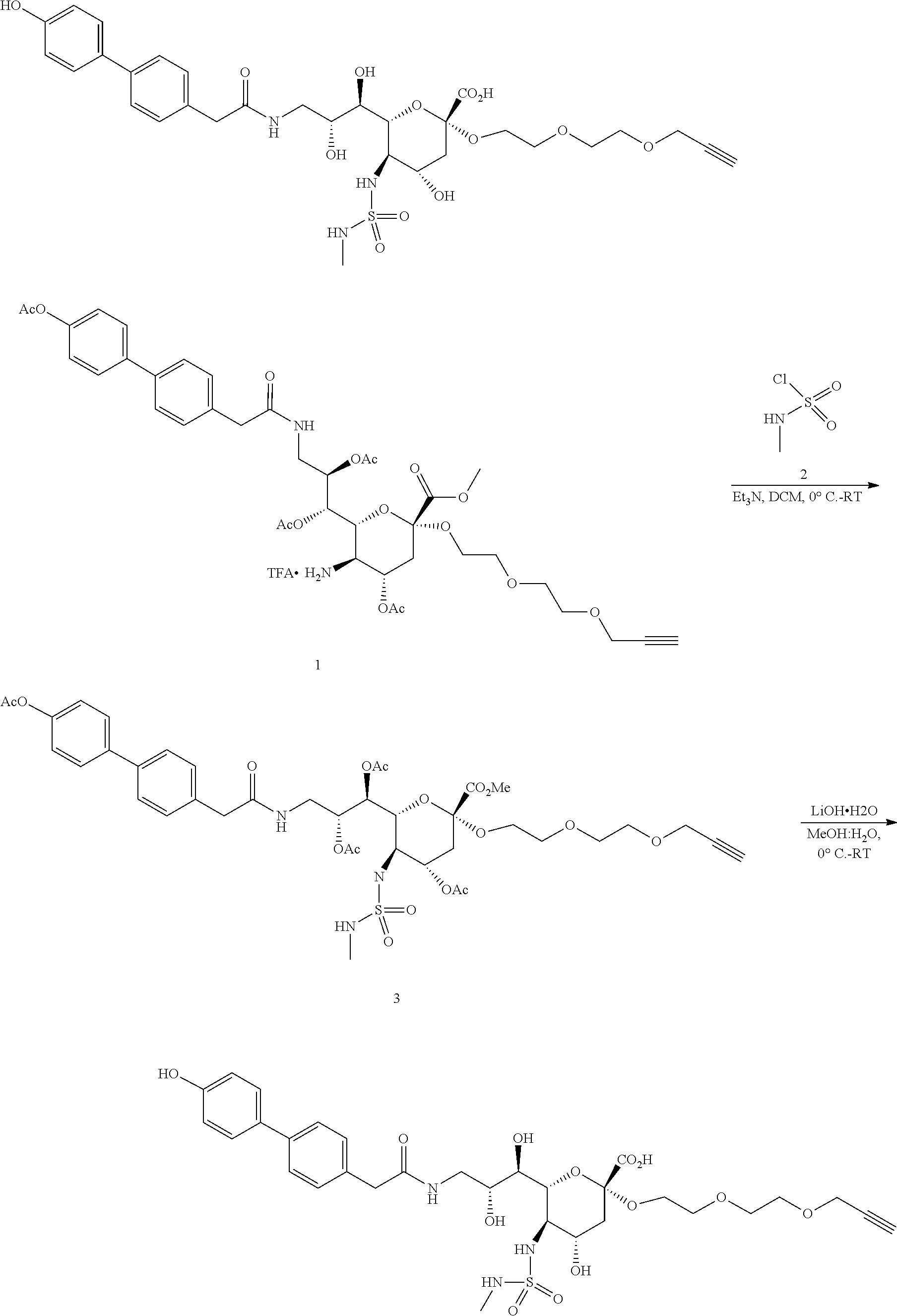

### Synthesis of (1R,2R)-1-((2R,3R,4S,6R)-4-acetoxy-3-(2-acetoxy-2-methylpropanamido)-6-(methoxycarbonyl)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (3)

To a solution of (1R,2R)-1-((2R,3R,4S,6R)-4-acetoxy-3-amino-6-(methoxycarbonyl)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (0.10 g, 0.13 mmol) in tetrahydrofuran (2.0 mL), and cooled at 0° C. To this solution, triethylamine (0.053 mL, 0.38 mmol) followed by methylsulfamoyl chloride (0.020 g, 0.15 mmol) was added slowly at 0° C. The reaction was stirred at 0° C. After completion, the mixture was concentrated and the crude residue obtained was purified by silica gel column chromatography using ethyl acetate in hexane to afford the desired product (1R,2R)-1-((2R,3R,4S,6R)-4-acetoxy-6-(methoxycarbonyl)-3-((N-methylsulfamoyl)amino)-6-(2-(2-(prop-2-yn-1-yloxy)ethoxy)ethoxy)tetrahydro-2H-pyran-2-yl)-3-(2-(4′-hydroxy-[1,1′-biphenyl]-4-yl)acetamido)propane-1,2-diyl diacetate (3). Yield: 0.112 g (49%), 50%; LCMS (ESI) m/z 878.2 [M+H]⁺.

### Synthesis of (2R,4S,5R,6R)-6-((1R,2R)-1,2-dihydroxy-3-(2-(4′-hydroxy-[1,1′-biphenyl]-4-yl)acetamido)propyl)-4-hydroxy-5-((N-methylsulfamoyl)amino)-2-(2-(2-(prop-2-yn-1-yloxy)ethoxy)ethoxy)tetrahydro-2H-pyran-2-carboxylic acid (Cpd. 225)

To a stirred solution of (1R,2R)-1-((2R,3R,4S,6R)-4-acetoxy-6-(methoxycarbonyl)-3-((N-methylsulfamoyl)amino)-6-(2-(2-(prop-2-yn-1-yloxy)ethoxy)ethoxy)tetrahydro-2H-pyran-2-yl)-3-(2-(4′-acetoxy-[1,1′-biphenyl]-4-yl)acetamido)propane-1,2-diyl diacetate (3, 0.112 g, 0.13 mmol) in methanol (3 mL), was added a solution of lithium hydroxide monohydrate (0.020 g, 0.76 mmol) in water (0.2 mL). The reaction mixture was stirred at room temperature for 6 h. After completion, the reaction mixture was treated with acidic resin (Dowex 50, H⁺) up to pH ˜6 and the suspension was filtered. The filtrate was concentrated. The crude residue obtained was purified by prep HPLC using acetonitrile in water (+0.1% FA) to afford the desired product (2R,4S,5R,6R)-6-((1R,2R)-1,2-dihydroxy-3-(2-(4′-hydroxy-[1,1′-biphenyl]-4-yl)acetamido)propyl)-4-hydroxy-5-((N-methylsulfamoyl)amino)-2-(2-(2-(prop-2-yn-1-yloxy)ethoxy)ethoxy)tetrahydro-2H-pyran-2-carboxylic acid (Cpd. 225). Yield: 0.013 g, 14.6%; LCMS m/z 696.30 [M+H]+;¹H NMR (400 MHz, methanol-d₄) δ 7.51 (d, J=8.0 Hz, 2H), 7.45 (d, J=8.8 Hz, 2H), 7.35 (d, J=8.0 Hz, 2H), 6.85 (d, J=8.8 Hz, 2H), 4.17 (d, J=2.4 Hz, 2H), 3.85-3.52 (m, 15H), 3.42-3.24 (m, 2H), 2.83 (t, J=2.4 Hz, 1H), 2.70 (dd, J=2.0 Hz, 6.8 Hz, 1H), 2.65 (s, 3H), 1.76 (t, J=12.4 Hz, 1H).

### 3-(2-(2-(((2R,4S,5R,6R)-6-((1R,2R)-3-(2-(4-chlorophenyl)acetamido)-1,2-dihydroxypropyl)-4-hydroxy-5-(2-hydroxyacetamido)-2-(2H-tetrazol-5-yl)tetrahydro-2H-pyran-2-yl)oxy)ethoxy)ethoxy)-N-(prop-2-yn-1-yl)propanamide (Compound 226)

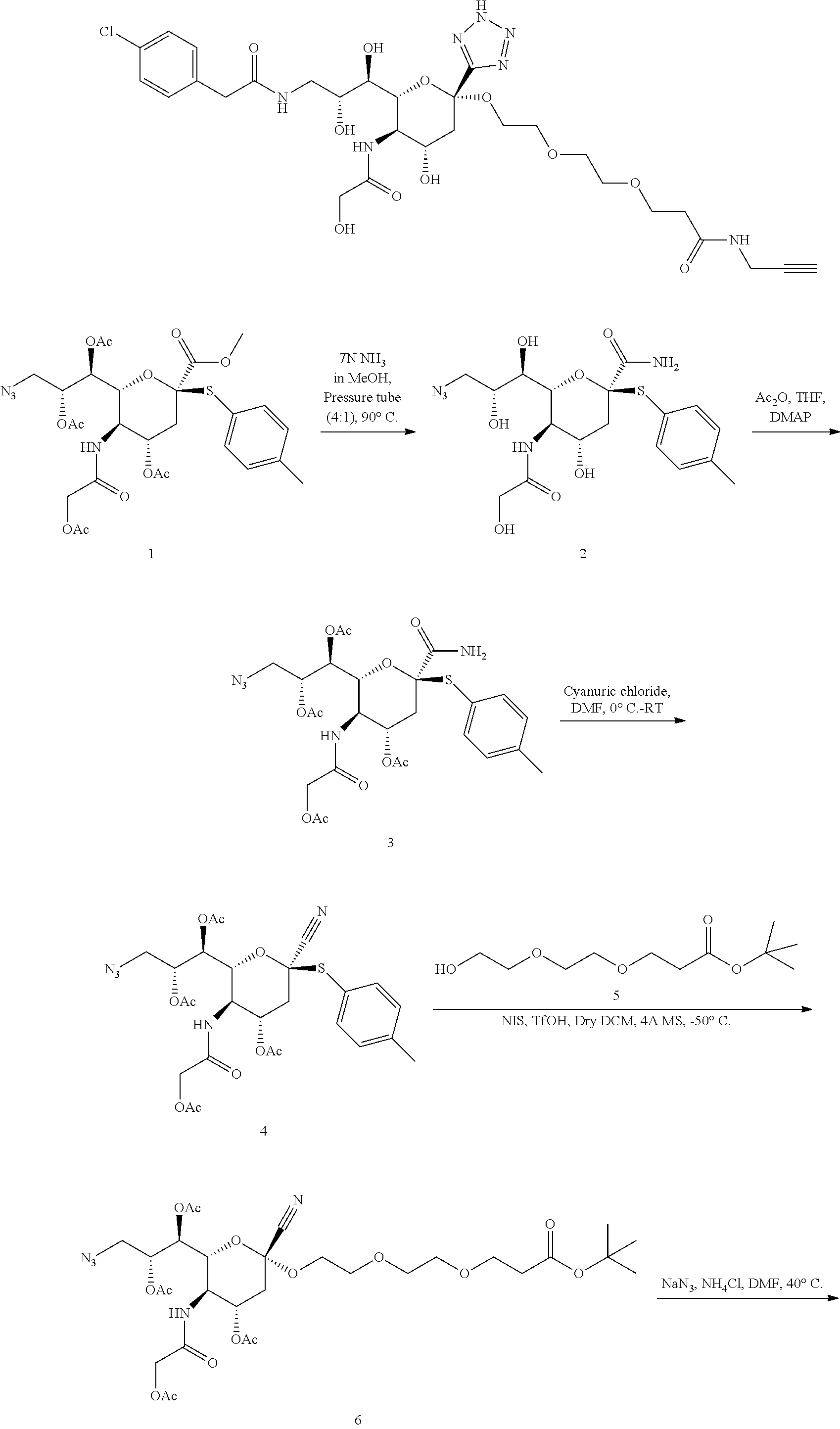

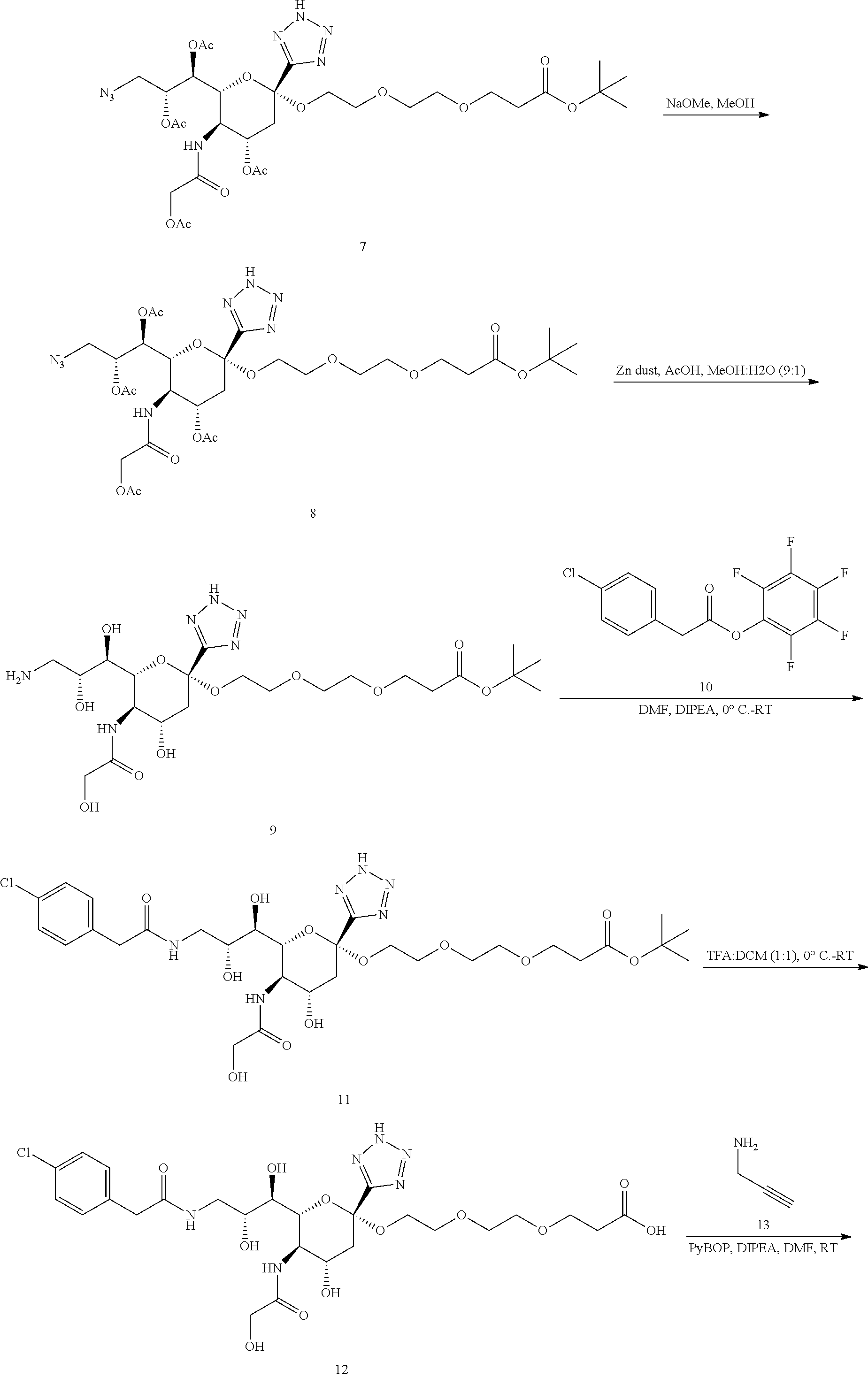

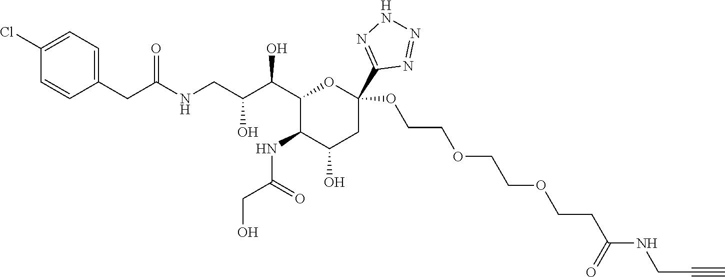 [SMILES: C#CCNC(=O)CCOCCOCCO[C@]1(c2nn[nH]n2)C[C@H](O)[C@@H](NC(=O)CO)[C@H]([C@H](O)[C@H](O)CNC(=O)Cc2ccc(Cl)cc2)O1]

### Synthesis of (2R,4S,5R,6R)-6-((1R,2R)-3-azido-1,2-dihydroxypropyl)-4-hydroxy-5-(2-hydroxyacetamido)-2-(p-tolylthio)tetrahydro-2H-pyran-2-carboxamide (2)

A solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-azidopropane-1,2-diyl diacetate (1, 5.0 g, 7.83 mmol) and ammonia in methanol (110 mL) was heated at 100° C. for 16 h in steel bomb. After completion, the reaction mixture was concentrated to afford the desired crude product which was triturated with ether and dried to afford the desired product (2R,4S,5R,6R)-6-((1R,2R)-3-azido-1,2-dihydroxypropyl)-4-hydroxy-5-(2-hydroxyacetamido)-2-(p-tolylthio)tetrahydro-2H-pyran-2-carboxamide (2) which was used for the next step without further purification. Yield: 4.0 g, Crude; LCMS (ESI) m/z 455.90 [M+H]⁺.

### Synthesis of (1R,2R)-1-((2R,3R,4S,6R)-4-acetoxy-3-(2-acetoxyacetamido)-6-carbamoyl-6-(p-tolylthio)tetrahydro-2H-pyran-2-yl)-3-azidopropane-1,2-diyl diacetate (3)

To a stirred solution of (2R,4S,5R,6R)-6-((1R,2R)-3-azido-1,2-dihydroxypropyl)-4-hydroxy-5-(2-hydroxyacetamido)-2-(p-tolylthio)tetrahydro-2H-pyran-2-carboxamide (2, 4.0 g, 5.88 mmol) in tetrahydrofuran (40 mL) was added acetic anhydride (5.56 mL, 58.8 mmol) and 4-methyl aminopyridine (0.79 g, 6.47 mmol) at 0° C. The resulting reaction mixture was stirred at room temperature for 14 h. After completion, the reaction mixture was concentrated and the residue was taken up in ethyl acetate and washed with brine. The organic layer was dried, concentrated and the crude residue obtained was purified by silica gel column chromatography using ethyl acetate in heptane to afford the desired product (1R,2R)-1-((2R,3R,4S,6R)-4-acetoxy-3-(2-acetoxyacetamido)-6-carbamoyl-6-(p-tolylthio)tetrahydro-2H-pyran-2-yl)-3-azidopropane-1,2-diyl diacetate (3). Yield: 2.3 g, 57.04%; LCMS m/z 622.90 [M−H]⁻.

### Synthesis of (1R,2R)-1-((2R,3R,4S,6R)-4-acetoxy-3-(2-acetoxyacetamido)-6-cyano-6-(p-tolylthio)tetrahydro-2H-pyran-2-yl)-3-azidopropane-1,2-diyl diacetate (4)

To a stirred solution of (1R,2R)-1-((2R,3R,4S,6R)-4-acetoxy-3-(2-acetoxyacetamido)-6-carbamoyl-6-(p-tolylthio)tetrahydro-2H-pyran-2-yl)-3-azidopropane-1,2-diyl diacetate (3, 2.0 g, 3.21 mmol) in N,N-dimethylformamide (20 mL), trichloro-1,3,5-triazine (2.96 g, 16.0 mmol) was added portion wise at 0° C. After complete addition the reaction mixture was stirred at room temperature for 3 h. After completion the reaction mixture was poured into cold water and extracted with ethyl acetate. The organic layer was washed with a solution of sodium bicarbonate then dried and concentrated. The crude residue obtained was purified by silica gel column chromatography using ethyl acetate in heptane to afford the desired product (1R,2R)-1-((2R,3R,4S,6R)-4-acetoxy-3-(2-acetoxyacetamido)-6-cyano-6-(p-tolylthio)tetrahydro-2H-pyran-2-yl)-3-azidopropane-1,2-diyl diacetate (4). Yield: 1.5 g, 74.14%; LCMS m/z 605.95 [M+H]⁺.

### Synthesis of (1R,2R)-1-((2R,3R,4S,6R)-4-acetoxy-3-(2-acetoxyacetamido)-6-(2-(2-(3-(tert-butoxy)-3-oxopropoxy)ethoxy)ethoxy)-6-cyanotetrahydro-2H-pyran-2-yl)-3-azidopropane-1,2-diyl diacetate (6)

A mixture of (1R,2R)-1-((2R,3R,4S,6R)-4-acetoxy-3-(2-acetoxyacetamido)-6-cyano-6-(p-tolylthio)tetrahydro-2H-pyran-2-yl)-3-azidopropane-1,2-diyl diacetate (4, 0.50 g, 0.83 mmol), tert-butyl 3-(2-(2-hydroxyethoxy)ethoxy)propanoate (5, 1.55 g, 12.10 mmol), and activated 4 Å powdered molecular sieves (0.5 g) in anhydrous dichloromethane (10 mL) was stirred at room temperature for 15 h. 1-iodopyrrolidine-2,5-dione (0.46 g, 2.06 mmol) and trifluoromethanesulfonic acid (0.072 mL, 0.83 mmol) solution in dichloromethane (1.0 mL) was added at −40° C. and the reaction was stirred at the same temperature for 1 h. After completion the reaction mixture was quenched by addition of triethyl amine (0.5 mL) and warmed up to room temperature. The reaction mixture was filtered. The filtrate was washed with saturated solution of sodium bicarbonate and dried, filtered and concentrated. The crude product was purified by silica gel column chromatography using ethyl acetate in heptane as eluents to afford the desired product (1R,2R)-1-((2R,3R,4S,6R)-4-acetoxy-3-(2-acetoxyacetamido)-6-(2-(2-(3-(tert-butoxy)-3-oxopropoxy)ethoxy)ethoxy)-6-cyanotetrahydro-2H-pyran-2-yl)-3-azidopropane-1,2-diyl diacetate (6). Yield: 0.35 g, 41.46%; ELSD-MS m/z 733.85 [M+H3O]⁺.

### Synthesis of (1R,2R)-1-((2R,3R,4S,6R)-4-acetoxy-3-(2-acetoxyacetamido)-6-(2-(2-(3-(tert-butoxy)-3-oxopropoxy)ethoxy)ethoxy)-6-(2H-tetrazol-5-yl)tetrahydro-2H-pyran-2-yl)-3-azidopropane-1,2-diyl diacetate (7)

To a stirred solution of (1R,2R)-1-((2R,3R,4S,6R)-4-acetoxy-3-(2-acetoxyacetamido)-6-(2-(2-(3-(tert-butoxy)-3-oxopropoxy)ethoxy)ethoxy)-6-cyanotetrahydro-2H-pyran-2-yl)-3-azidopropane-1,2-diyl diacetate (6, 1.0 g, 1.33 mmol) in N,N-dimethylformamide (10 mL) was added sodium azide (0.43 g, 6.64 mmol) and ammonium chloride (0.35 g, 6.64 mmol) at room temperature and stirred at 50° C. for 24 h. The reaction was monitored by TLC and LCMS. After completion, the reaction mixture was concentrated. The crude residue obtained was purified by silica gel column chromatography and desired compound was eluting at 0-20% methanol in dichloromethane with 0.1% acetic acid to afford the desired product (1R,2R)-1-((2R,3R,4S,6R)-4-acetoxy-3-(2-acetoxyacetamido)-6-(2-(2-(3-(tert-butoxy)-3-oxopropoxy)ethoxy)ethoxy)-6-(2H-tetrazol-5-yl)tetrahydro-2H-pyran-2-yl)-3-azidopropane-1,2-diyl diacetate (7) as a brown sticky liquid. Yield: 0.50 g, 49.15%; ELSD-MS m/z 775.70 [M+H3O]⁺.

### Synthesis of tert-butyl 3-(2-(2-(((2R,4S,5R,6R)-6-((1R,2R)-3-azido-1,2-dihydroxypropyl)-4-hydroxy-5-(2-hydroxyacetamido)-2-(2H-tetrazol-5-yl)tetrahydro-2H-pyran-2-yl)oxy)ethoxy)ethoxy)propanoate (8)

To a stirred solution of (1R,2R)-1-((2R,3R,4S,6R)-4-acetoxy-3-(2-acetoxyacetamido)-6-(2-(2-(3-(tert-butoxy)-3-oxopropoxy)ethoxy)ethoxy)-6-(2H-tetrazol-5-yl)tetrahydro-2H-pyran-2-yl)-3-azidopropane-1,2-diyl diacetate (7, 0.50 g, 0.66 mmol) in methanol (10.0 mL) was added sodium methoxide (0.18 g, 3.29 mmol) at 0° C. The reaction mixture was stirred at room temperature for 4 h. After completion the reaction mixture was treated with Dowex 50-H⁺ to pH˜6 and the suspension was filtered. The filtrate was concentrated. The crude residue obtained was triturated to afford tert-butyl 3-(2-(2-(((2R,4S,5R,6R)-6-((1R,2R)-3-azido-1,2-dihydroxypropyl)-4-hydroxy-5-(2-hydroxyacetamido)-2-(2H-tetrazol-5-yl)tetrahydro-2H-pyran-2-yl)oxy)ethoxy)ethoxy)propanoate (8). Yield: 0.40 g, 90.44%; ELSD-MS m/z 590.90 [M+H]⁺.

### Synthesis of tert-butyl 3-(2-(2-(((2R,4S,5R,6R)-6-((1R,2R)-3-amino-1,2-dihydroxypropyl)-4-hydroxy-5-(2-hydroxyacetamido)-2-(2H-tetrazol-5-yl)tetrahydro-2H-pyran-2-yl)oxy)ethoxy)ethoxy)propanoate (9)

To a stirred solution of tert-butyl 3-(2-(2-(((2R,4S,5R,6R)-6-((1R,2R)-3-azido-1,2-dihydroxypropyl)-4-hydroxy-5-(2-hydroxyacetamido)-2-(2H-tetrazol-5-yl)tetrahydro-2H-pyran-2-yl)oxy)ethoxy)ethoxy)propanoate (8, 0.50 g, 0.85 mmol) in methanol (8.0 mL) and water (2.0 mL), zinc dust (0.55 g, 8.47 mmol) and acetic acid (0.24 mL, 4.23 mmol) was added portion wise at 0° C. and the reaction mixture was stirred at room temperature for 2 h. After completion the reaction mixture was filtered. The filtrate was concentrated. The crude residue obtained was triturated with diethyl ether to afford the desired product tert-butyl 3-(2-(2-(((2R,4S,5R,6R)-6-((1R,2R)-3-amino-1,2-dihydroxypropyl)-4-hydroxy-5-(2-hydroxyacetamido)-2-(2H-tetrazol-5-yl)tetrahydro-2H-pyran-2-yl)oxy)ethoxy)ethoxy)propanoate (9). Yield: 0.30 g, 62.76%; ELSD-MS m/z 564.90 [M+H]⁺.

### Synthesis of tert-butyl 3-(2-(2-(((2R,4S,5R,6R)-6-((1R,2R)-3-(2-(4-chlorophenyl)acetamido)-1,2-dihydroxypropyl)-4-hydroxy-5-(2-hydroxyacetamido)-2-(2H-tetrazol-5-yl)tetrahydro-2H-pyran-2-yl)oxy)ethoxy)ethoxy)propanoate (11)

To a stirred solution of tert-butyl 3-(2-(2-(((2R,4S,5R,6R)-6-((1R,2R)-3-amino-1,2-dihydroxypropyl)-4-hydroxy-5-(2-hydroxyacetamido)-2-(2H-tetrazol-5-yl)tetrahydro-2H-pyran-2-yl)oxy)ethoxy)ethoxy)propanoate (9, 0.15 g, 0.27 mmol) in N,N-dimethylformamide (3.0 mL), N,N-diisopropylethylamine (0.23 mL, 1.33 mmol) and perfluorophenyl 2-(4-chlorophenyl)acetate (10, 0.07 g, 0.21 mmol) was added at 0° C. and the reaction mixture was stirred at room temperature for 4 h. After completion the reaction mixture was concentrated to afford the desired crude product tert-butyl 3-(2-(2-(((2R,4S,5R,6R)-6-((1R,2R)-3-(2-(4-chlorophenyl)acetamido)-1,2-dihydroxypropyl)-4-hydroxy-5-(2-hydroxyacetamido)-2-(2H-tetrazol-5-yl)tetrahydro-2H-pyran-2-yl)oxy)ethoxy)ethoxy)propanoate (11). The crude was used for the next step without further purification. Yield: 0.12 g, Crude; LCMS (ESI) m/z 717.40 [M+H]⁺.

### Synthesis of 3-(2-(2-(((2R,4S,5R,6R)-6-((1R,2R)-3-(2-(4-chlorophenyl)acetamido)-1,2-dihydroxypropyl)-4-hydroxy-5-(2-hydroxyacetamido)-2-(2H-tetrazol-5-yl)tetrahydro-2H-pyran-2-yl)oxy)ethoxy)ethoxy)propanoic acid (12)

To a stirred solution of tert-butyl 3-(2-(2-(((2R,4S,5R,6R)-6-((1R,2R)-3-(2-(4-chlorophenyl)acetamido)-1,2-dihydroxypropyl)-4-hydroxy-5-(2-hydroxyacetamido)-2-(2H-tetrazol-5-yl)tetrahydro-2H-pyran-2-yl)oxy)ethoxy)ethoxy)propanoate (11, 0.17 g, 0.24 mmol), in anhydrous dichloromethane (3.0 mL), trifluoroacetic acid (1.0 mL) was added at 0° C. and the reaction mixture was stirred at room temperature for 3 h. After completion the solvent was concentrated and the crude residue obtained was purified by Prep-HPLC using acetonitrile in water (+0.1% TFA) as eluents to afford the desired product 3-(2-(2-(((2R,4S,5R,6R)-6-((1R,2R)-3-(2-(4-chlorophenyl)acetamido)-1,2-dihydroxypropyl)-4-hydroxy-5-(2-hydroxyacetamido)-2-(2H-tetrazol-5-yl)tetrahydro-2H-pyran-2-yl)oxy)ethoxy)ethoxy)propanoic acid (12). Yield: 0.06 g, Crude; LCMS (ESI) m/z 661.32 [M+H]⁺.

### Synthesis of 3-(2-(2-(((2R,4S,5R,6R)-6-((1R,2R)-3-(2-(4-chlorophenyl)acetamido)-1,2-dihydroxypropyl)-4-hydroxy-5-(2-hydroxyacetamido)-2-(2H-tetrazol-5-yl)tetrahydro-2H-pyran-2-yl)oxy)ethoxy)ethoxy)-N-(prop-2-yn-1-yl)propanamide (Cpd. 226)

To a stirred solution of 3-(2-(2-(((2R,4S,5R,6R)-6-((1R,2R)-3-(2-(4-chlorophenyl)acetamido)-1,2-dihydroxypropyl)-4-hydroxy-5-(2-hydroxyacetamido)-2-(2H-tetrazol-5-yl)tetrahydro-2H-pyran-2-yl)oxy)ethoxy)ethoxy)propanoic acid (12, 0.04 g, 0.06 mmol) and propargyl amine (13, 0.004 mL, 0.06 mmol) in N,N-dimethylformamide (2.0 mL) was added benzotriazol-1-yloxytripyrrolidinophosphonium hexafluorophosphate (0.063 g, 0.12 mmol) was added at 0° C. After 10 min ethylbis(propan-2-yl)amine (0.03 mL, 0.18 mmol) was added dropwise and the reaction mixture was stirred at room temperature for 24 h. After completion the reaction mixture was concentrated and the crude residue obtained was purified by Prep-HPLC using acetonitrile in water (+0.1% TFA) as eluents to afford the desired product 3-(2-(2-(((2R,4S,5R,6R)-6-((1R,2R)-3-(2-(4-chlorophenyl)acetamido)-1,2-dihydroxypropyl)-4-hydroxy-5-(2-hydroxyacetamido)-2-(2H-tetrazol-5-yl)tetrahydro-2H-pyran-2-yl)oxy)ethoxy)ethoxy)-N-(prop-2-yn-1-yl)propanamide (Cpd. 226). Yield: 0.028 g, 66.28%; LCMS (ESI) m/z 698.40 [M+H]⁺. ¹H NMR (400 MHz, Methanol-d₄): δ 7.32-7.27 (m, 4H), 4.29 (td, J=10.8 & 4.8 Hz, 1H), 4.18 (d, J=10.8 Hz, 1H), 4.06 (d, J=4.0 Hz, 2H), 4.02-3.97 (m, 1H), 3.95 (d, J=4.0 Hz, 2H), 3.89-3.85 (m, 1H), 3.75-3.58 (m, 10H), 3.54 (s, 2H), 3.49-3.35 (m, 1H), 3.30-3.26 (m, 1H), 3.21-3.13 (m, 1H), 2.61 (dd, J=12.8 & 4.8 Hz, 1H), 2.57 (t, J=2.8 Hz, 1H), 2.46 (t, J=2.8 Hz, 2H), 1.66 (dd, J=12.8 & 11.2 Hz, 1H).

### (2R,4S,5R,6R)-6-((1R,2R)-1,2-dihydroxy-3-(2-(3-hydroxyphenyl)acetamido)propyl)-4-hydroxy-5-(2-hydroxyacetamido)-2-((7-oxo-7-(propylamino)heptyl)oxy)tetrahydro-2H-pyran-2-carboxylic acid (Compound 207)

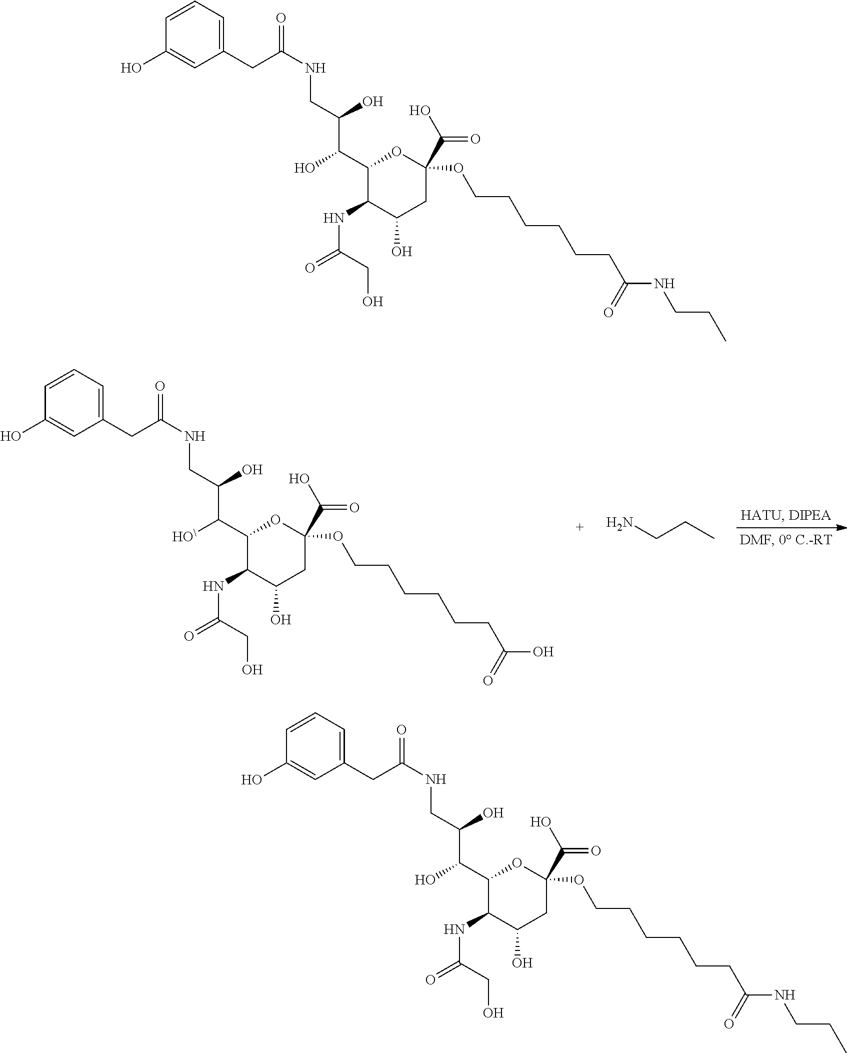

### Synthesis of (2R,4S,5R,6R)-6-((1R,2R)-1,2-dihydroxy-3-(2-(3-hydroxyphenyl)acetamido)propyl)-4-hydroxy-5-(2-hydroxyacetamido)-2-((7-oxo-7-(propylamino)heptyl)oxy)tetrahydro-2H-pyran-2-carboxylic acid (Cpd. 207)

To a stirred solution of (2R,4S,5R,6R)-2-((6-carboxyhexyl)oxy)-6-((1R,2R)-1,2-dihydroxy-3-(2-(3-hydroxyphenyl)acetamido)propyl)-4-hydroxy-5-(2-hydroxyacetamido)tetrahydro-2H-pyran-2-carboxylic acid (SME40070 [Cpd. 143], 20.0 mg, 34.1 umol) in N,N-dimethylformamide (0.68 mL) was added HATU (14.5 mg, 38.2 umol) at room temperature. After 10 minutes, N,N-diisopropylethylamine (17.8 uL, 102 umol) was added dropwise at 0° C., followed by propylamine (2.8 uL, 34.1 umol). The resulting reaction mixture was stirred at room temperature for 20 min. After completion the reaction mixture was purified by prep HPLC using water and acetonitrile as eluents to afford the desired (Cpd. 207) as white solid. Yield: 11.5 mg, 54%; LCMS (ESI) m/z 626.4 [M−H]⁻, ¹H NMR (500 MHz, DMSO-d₆): 9.27 (s, br, 1H), 7.89 (t, J=5.5 Hz, 1H), 7.85 (d, J=7.5 Hz, 1H), 7.73 (t, J=5.5 Hz, 1H), 7.04 (t, J=8.0 Hz, 1H), 6.67-6.65 (m, 1H), 6.60-6.57 (m, 1H), 3.91-3.83 (m, 3H), 3.82-3.78 (m, 1H), 3.67-3.60 (m, 4H), 3.56-3.26 (m, 6H), 3.21-3.19 (m, 2H), 2.99-2.95 (m, 3H), 2.94-2.90 (m, 1H), 2.02 (t, J=7.5 Hz, 2H), 1.54-1.51 (m, 1H), 1.49-1.41 (m, 4H), 1.38 (tq, J=7.5 Hz, 2H), 1.27-1.19 (m, 4H), 0.82 (t, J=7.5 Hz, 3H).

### (2R,4S,5R,6R)-6-((1R,2R)-3-(3-chlorobenzamido)-1,2-dihydroxypropyl)-2-((6-((3,4-dioxo-2-((2-(2-(prop-2-yn-1-yloxy)ethoxy)ethyl)amino)cyclobut-1-en-1-yl)amino)hexyl)oxy)-4-hydroxy-5-(2-hydroxyacetamido)tetrahydro-2H-pyran-2-carboxylic acid (Compound 227)

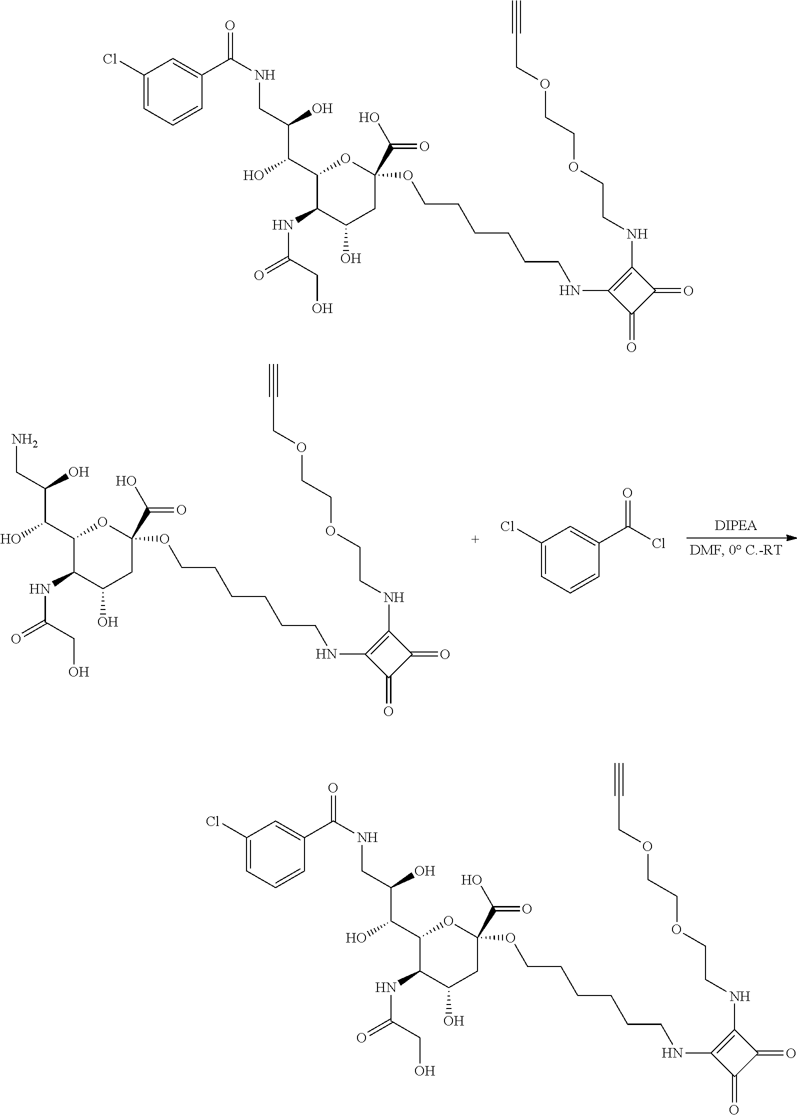 [SMILES: C#CCOCCOCCNc1c(NCCCCCCO[C@]2(C(=O)O)C[C@H](O)[C@@H](NC(=O)CO)[C@H]([C@H](O)[C@H](O)CNC(=O)c3cccc(Cl)c3)O2)c(=O)c1=O ; C#CCOCCOCCNc1c(NCCCCCCO[C@]2(C(=O)O)C[C@H](O)[C@@H](NC(=O)CO)[C@H]([C@H](O)[C@H](O)CN)O2)c(=O)c1=O ; O=C(Cl)c1cccc(Cl)c1 ; C#CCOCCOCCNc1c(NCCCCCCO[C@]2(C(=O)O)C[C@H](O)[C@@H](NC(=O)CO)[C@H]([C@H](O)[C@H](O)CNC(=O)c3cccc(Cl)c3)O2)c(=O)c1=O]

### Synthesis of (2R,4S,5R,6R)-6-((1R,2R)-3-(3-chlorobenzamido)-1,2-dihydroxypropyl)-2-((6-((3,4-dioxo-2-((2-(2-(prop-2-yn-1-yloxy)ethoxy)ethyl)amino)cyclobut-1-en-1-yl)amino)hexyl)oxy)-4-hydroxy-5-(2-hydroxyacetamido)tetrahydro-2H-pyran-2-carboxylic acid (Cpd. 227)

To a stirred solution of (2R,4S,5R,6R)-6-((1R,2R)-3-amino-1,2-dihydroxypropyl)-2-((6-((3,4-dioxo-2-((2-(2-(prop-2-yn-1-ylxy)ethoxy)ethyl)amino)cyclobut-1-en-1-yl)amino)hexyl)oxy)-4-hydroxy-5-(2-hydroxyacetamido)tetrahydro-2H-pyran-2-carboxylic acid (SME40289, 80.0 mg, 124 umol) in N,N-dimethylformamide (2.0 mL) was added 3-chlorobenzoyl chloride (16.5 uL, 130 umol) at room temperature. After 10 minutes, N,N-diisopropylethylamine (64.8 uL, 372 umol) was added dropwise at 0° C. The resulting reaction mixture was stirred at room temperature for 2 h. After completion, the reaction mixture was purified by prep HPLC using water and acetonitrile as eluents to afford the desired product (Cpd. 227) as a white solid. Yield: 55 mg, 57%; LCMS (ESI) m/z 781.4 [M−H]⁻. ¹H NMR (500 MHz, Methanol-d₄): 7.85 (t, J=2.0 Hz, 1H), 7.76 (d, J=8.0 Hz, 1H), 7.54 (dt, J=8.0 Hz & 2.0 Hz, 1H), 7.45 (t, J=8.0 Hz, 1H), 4.17 (d, J=2.5 Hz, 2H), 4.04-4.00 (m, 1H), 4.02 (s, 2H), 3.85-3.82 (m, 2H), 3.81-3.74 (m, 5H), 3.69-3.64 (m, 4H), 3.64-3.62 (m, 2H), 3.59 (m, 2H), 3.52-3.40 (m, 3H), 2.85 (t, J=2.5 Hz, 1H), 2.71 (dd, J=12.5 Hz & 4.0 Hz, 1H), 1.76-1.71 (m, 1H), 1.61-1.55 (m, 4H), 1.42-1.38 (m, 4H).

### (2R,4S,5R,6R)-6-((1R,2R)-3-(3-chlorobenzamido)-1,2-dihydroxypropyl)-2-((6-((3,4-dioxo-2-((2-(2-((1-(15-oxo-15-(perfluorophenoxy)-3,6,9,12-tetraoxapentadecyl)-1H-1,2,3-triazol-4-yl)methoxy)ethoxy)ethyl)amino)cyclobut-1-en-1-yl)amino)hexyl)oxy)-4-hydroxy-5-(2-hydroxyacetamido)tetrahydro-2H-pyran-2-carboxylic acid (Compound 227P)

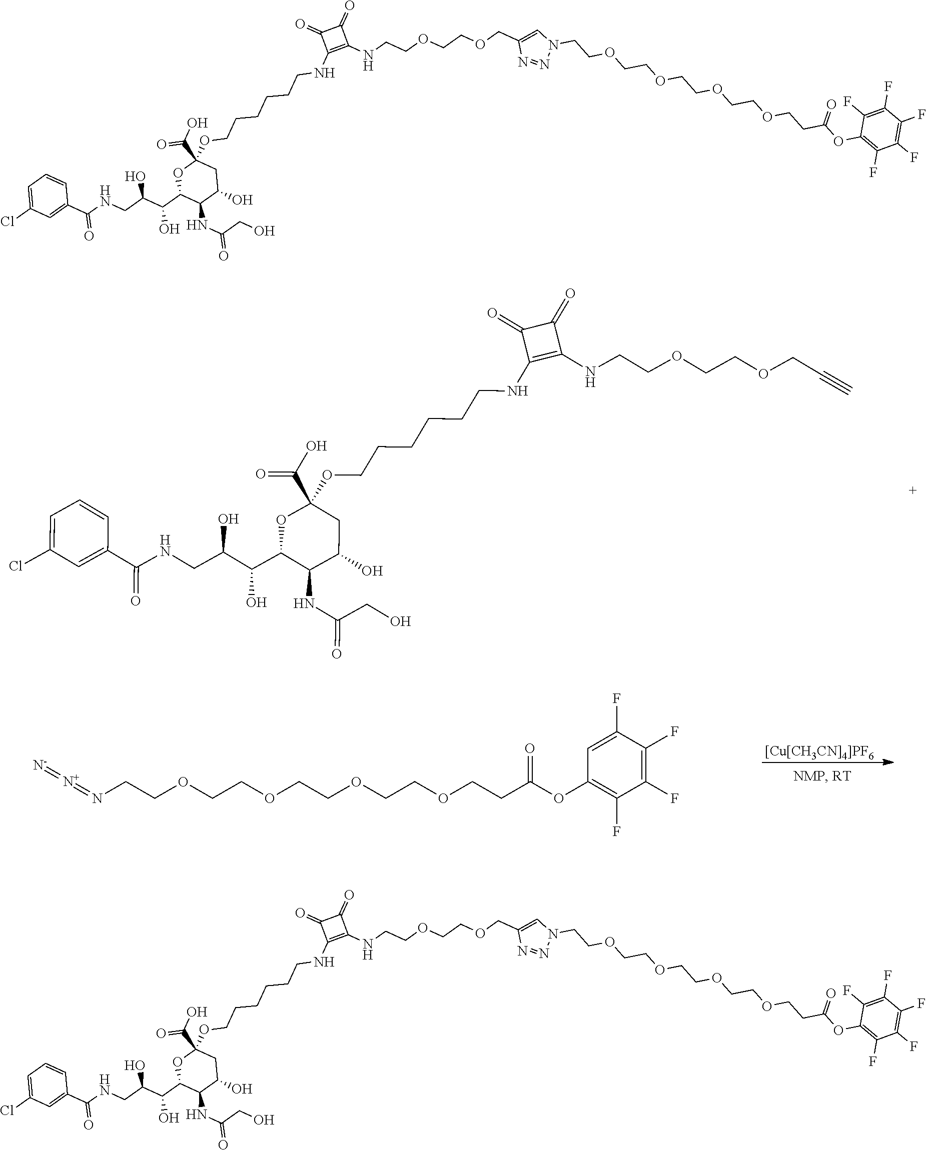

### Synthesis of (2R,4S,5R,6R)-6-((1R,2R)-3-(3-chlorobenzamido)-1,2-dihydroxypropyl)-2-((6-((3,4-dioxo-2-((2-(2-((1-(15-oxo-15-(perfluorophenoxy)-3,6,9,12-tetraoxapentadecyl)-1H-1,2,3-triazol-4-yl)methoxy)ethoxy)ethyl)amino)cyclobut-1-en-1-yl)amino)hexyl)oxy)-4-hydroxy-5-(2-hydroxyacetamido)tetrahydro-2H-pyran-2-carboxylic acid (227P)

To a solution of (2R,4S,5R,6R)-6-((1R,2R)-3-(3-chlorobenzamido)-1,2-dihydroxypropyl)-2-((6-((3,4-dioxo-2-((2-(2-(prop-2-yn-1-yloxy)ethoxy)ethyl)amino)cyclobut-1-en-1-yl)amino)hexyl)oxy)-4-hydroxy-5-(2-hydroxyacetamido)tetrahydro-2H-pyran-2-carboxylic acid (227, 30.4 mg, 38.8 umol) in anhydrous NMP (518 uL) was added Azido-PEG4-PFP-ester (19.5 mg, 42.7 umol) followed by Tetrakis(acetonitrile)copper(l) hexafluorophosphate (36.2 mg, 97.0 umol). The vial was sealed and the mixture was stirred for 2 h at room temperature. The mixture was filtered through a 0.45 μm PTFE filter. A few drops of acetic acid were added to the filtrate. The filtrate was purified by preparatory HPLC using acetonitrile and water (+0.05% TFA) as eluents. The fractions containing the desired product were lyophilized to afford 227P as a white solid. Yield: 24.8 mg, 52%; LCMS (ESI) m/z 1240 [M+H]⁺. ¹H NMR (500 MHz, DMSO-d₆): 8.41 (t, J=5.5 Hz, 1H), 8.03 (s, 1H), 7.90 (t, J=4.0 Hz, 1H), 7.86 (d, J=8.0 Hz, 1H), 7.82-7.80 (m, 1H), 7.58-7.56 (m, 1H), 7.48 (t, J=8.0 Hz, 1H), 7.39 (s, br, 2H), 5.52 (s, br, 1H), 4.96 (s, br, 1H), 4.75 (s, br, 1H), 4.51 (s, 2H), 4.50 (t, J=5.5 Hz, 2H), 4.50 (s, 2H), 4.36 (s, br, 1H), 3.91-3.84 (m, 2H), 3.84-3.79 (m, 2H), 3.79 (t, J=5.5 Hz, 2H), 3.75 (t, J=5.5 Hz, 2H), 3.69-3.58 (m, 6H), 3.56-3.45 (m, 21H), 3.27-3.20 (m, 3H), 3.01 (t, J=6.0 Hz, 2H), 1.52 (t, J=12.0 Hz, 1H), 1.49-1.43 (m, 4H), 1.28-1.24 (m, 4H).

### (2R,4S,5R,6R)-6-((1R,2R)-3-(3-chlorobenzamido)-1,2-dihydroxypropyl)-2-((6-((2-((2-(2-((1-(18-(2,5-dioxo-2,5-dihydro-1H-pyrrol-1-yl)-15-oxo-3,6,9,12-tetraoxa-16-azaoctadecyl)-1H-1,2,3-triazol-4-yl)methoxy)ethoxy)ethyl)amino)-3,4-dioxocyclobut-1-en-1-yl)amino)hexyl)oxy)-4-hydroxy-5-(2-hydroxyacetamido)tetrahydro-2H-pyran-2-carboxylic acid (Compound 227M)

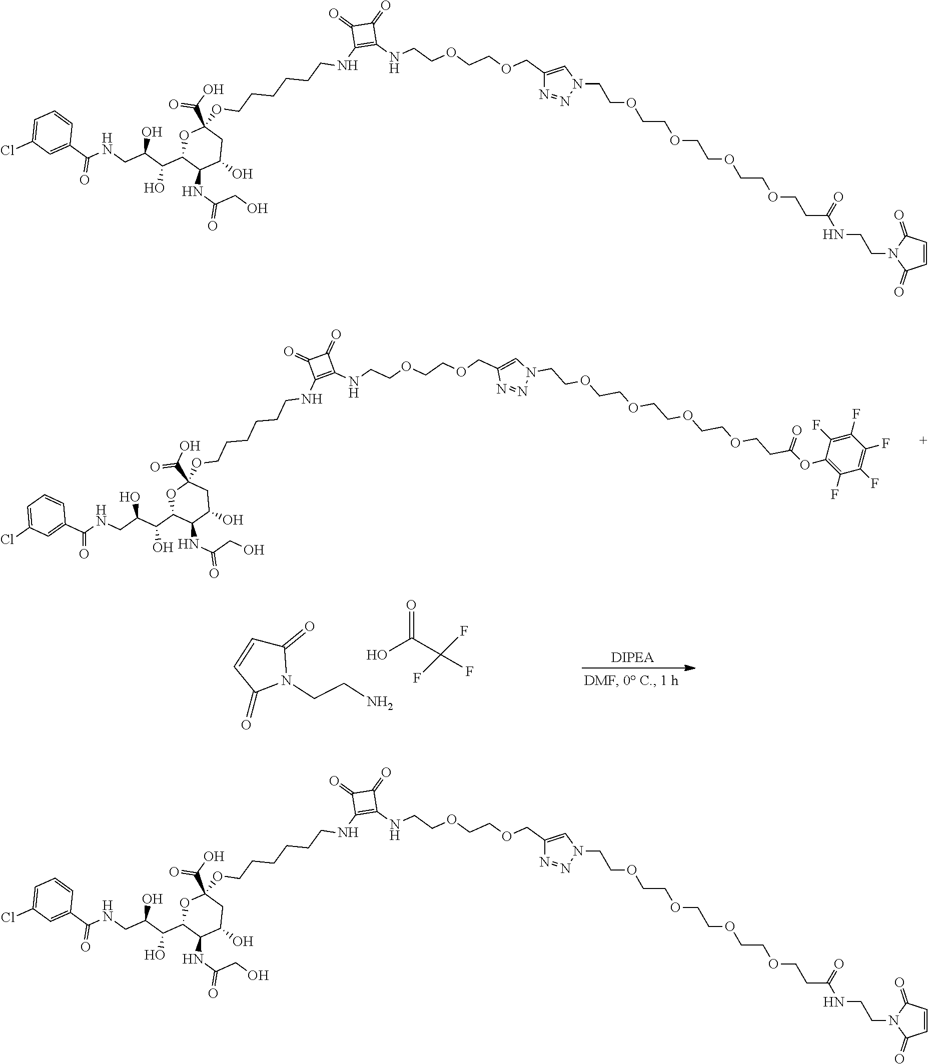

### Synthesis of (2R,4S,5R,6R)-6-((1R,2R)-3-(3-chlorobenzamido)-1,2-dihydroxypropyl)-2-((6-((2-((2-(2-((1-(18-(2,5-dioxo-2,5-dihydro-1-pyrrol-1-yl)-15-oxo-3,6,9,12-tetraoxa-16-azaoctadecyl)-1H-1,2,3-triazol-4-yl)methoxy)ethoxy)ethyl)amino)-3,4-dioxocyclobut-1-en-1-yl)amino)hexyl)oxy)-4-hydroxy-5-(2-hydroxyacetamido)tetrahydro-2H-pyran-2-carboxylic acid (227M)

To a solution of (2R,4S,5R,6R)-6-((1R,2R)-3-(3-chlorobenzamido)-1,2-dihydroxypropyl)-2-((6-((3,4-dioxo-2-((2-(2-((1-(15-oxo-15-(perfluorophenoxy)-3,6,9,12-tetraoxapentadecyl)-1H-1,2,3-triazol-4-yl)methoxy)ethoxy)ethyl)amino)cyclobut-1l-en-1-yl)amino)hexyl)oxy)-4-hydroxy-5-(2-hydroxyacetamido)tetrahydro-2H-pyran-2-carboxylic acid (227P, 19.8 mg, 16.0 umol) in DMF (400 uL) was added n-(2-aminoethyl)maleimide trifluoroacetate salt (4.27 mg, 16.8 umol). The mixture was cooled down to 0° C. and stirred for 10 min. DIEA (8.36 uL, 48.0 umol) was added and the mixture was stirred for 1 h at 0° C. The reaction mixture was filtered and purified by preparatory HPLC using acetonitrile and water (+0.05% TFA) as eluents. The fractions containing the desired product were combined and lyophilized to afford 227M as a white solid. Yield: 14.8 mg, 77%; LCMS (ESI) m/z 1196 [M+H]⁺. ¹H NMR (500 MHz, DMSO-d₆): 8.42 (t, J=5.5 Hz, 1H), 8.04 (s, 1H), 7.93 (t, J=6.0 Hz, 1H), 7.90 (t, J=4.0 Hz, 1H), 7.86 (d, J=8.0 Hz, 1H), 7.82-7.80 (m, 1H), 7.58-7.56 (m, 1H), 7.48 (t, J=8.0 Hz, 1H), 7.39 (s, br, 2H), 6.99 (s, 2H), 5.50 (s, br, 1H), 4.96 (s, br, 1H), 4.75 (s, br, 1H), 4.51 (s, 2H), 4.50 (t, J=5.5 Hz, 2H), 4.50 (s, 2H), 4.37 (s, br, 1H), 3.91-3.84 (m, 2H), 3.84-3.78 (m, 2H), 3.80 (t, J=5.5 Hz, 2H), 3.69-3.58 (m, 6H), 3.57-3.51 (m, 10H), 3.48-3.42 (m, 16H), 3.29-3.24 (m, 2H), 3.19-3.16 (m, 4H), 2.21 (t, J=6.5 Hz, 2H), 1.52 (t, J=12.0 Hz, 1H), 1.49-1.44 (m, 4H), 1.28-1.24 (m, 4H).

### (2R,4S,5R,6R)-6-((1R,2R)-3-(2-(4-chlorophenyl)acetamido)-1,2-dihydroxypropyl)-2-((2,19-dioxo-6,9,12,15-tetraoxa-3,18-diazatetracosan-24-yl)oxy)-4-hydroxy-5-(2-hydroxyacetamido)tetrahydro-2H-pyran-2-carboxylic acid (Compound 228)

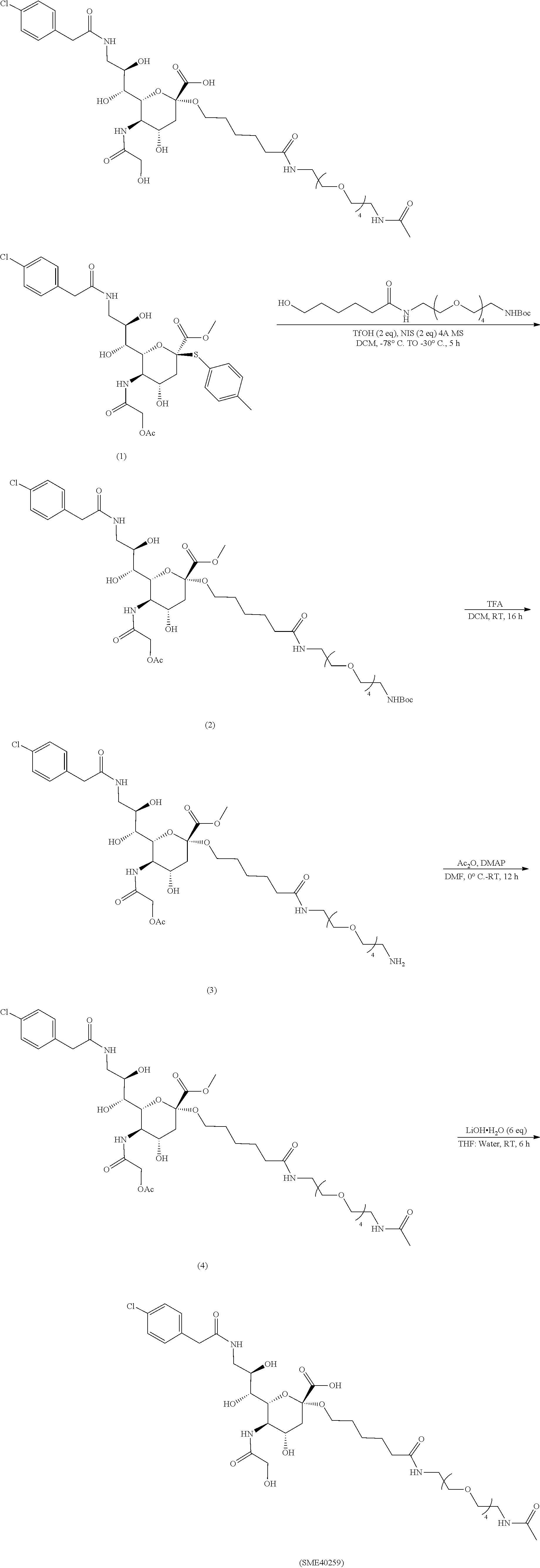

### Synthesis of methyl (2R,4S,5R,6R)-5-(2-acetoxyacetamido)-6-((1R,2R)-3-(2-(4-chlorophenyl)acetamido)-1,2-dihydroxypropyl)-2-((2,2-dimethyl-4,21-dioxo-3,8,11,14,17-pentaoxa-5,20-diazahexacosan-26-yl)oxy)-4-hydroxytetrahydro-2H-pyran-2-carboxylate (2)

A mixture of methyl (2R,4S,5R,6R)-5-(2-acetoxyacetamido)-6-((1R,2R)-3-(2-(4-chlorophenyl)acetamido)-1,2-dihydroxypropyl)-4-hydroxy-2-(p-tolylthio)tetrahydro-2H-pyran-2-carboxylate (1, 120 mg, 188 umol), tert-butyl (21-hydroxy-16-oxo-3,6,9,12-tetraoxa-15-azahenicosyl)carbamate (127 mg, 282 umol), activated 4 Å powdered molecular sieves (1.0 g) in anhydrous dichloromethane (3.76 mL) was stirred at room temperature for 1 h under nitrogen. The solution was cooled to −78° C., N-iodosuccinimide (84.5 mg, 376 umol) was added portion wise, followed by trifluoromethanesulfonic acid (33.2 uL, 376 umol), and the resulting mixture was stirred for 5 h from −78° C. to −30° C. After completion, the reaction mixture was quenched with N,N-diisopropylethylamine (196 uL) and the reaction was stirred for another 30 min at −30° C. before warming to room temperature. The reaction mixture was filtered, the filtrate was washed with saturated sodium thiosulfate solution, dried over sodium sulfate, filtered, and concentrated. The crude residue was purified by prep HPLC using water and acetonitrile as eluents to afford the desired product (2) as a colorless oily solid. Yield: 88 mg, 49%; LCMS (ESI) m/z 965 [M+H]⁺.

### Synthesis of methyl (2R,4S,5R,6R)-5-(2-acetoxyacetamido)-2-((1-amino-16-oxo-3,6,9,12-tetraoxa-15-azahenicosan-21-yl)oxy)-6-((1R,2R)-3-(2-(4-chlorophenyl)acetamido)-1,2-dihydroxypropyl)-4-hydroxytetrahydro-2H-pyran-2-carboxylate (3)

To a stirred solution of methyl (2R,4S,5R,6R)-5-(2-acetoxyacetamido)-6-((1R,2R)-3-(2-(4-chlorophenyl)acetamido)-1,2-dihydroxypropyl)-2-((2,2-dimethyl-4,21-dioxo-3,8,11,14,17-pentaoxa-5,20-diazahexacosan-26-yl)oxy)-4-hydroxytetrahydro-2H-pyran-2-carboxylate (2, 85.0 mg, 88.0 umol) in anhydrous dichloromethane (1.76 mL) was added trifluoroacetic acid (33.7 uL, 440 umol) at 0° C. The reaction mixture was stirred at room temperature for 16 h. After completion, the reaction mixture was concentrated and purified by prep HPLC using water and acetonitrile as eluents to afford the desired product (3) as a colorless oily solid. Yield: 69 mg, 91%; LCMS (ESI) m/z 865 [M+H]⁺.

### Synthesis of methyl (2R,4S,5R,6R)-5-(2-acetoxyacetamido)-6-((1R,2R)-3-(2-(4-chlorophenyl)acetamido)-1,2-dihydroxypropyl)-2-((2,19-dioxo-6,9,12,15-tetraoxa-3,18-diazatetracosan-24-yl)oxy)-4-hydroxytetrahydro-2H-pyran-2-carboxylate (4)

To a stirred solution of methyl (2R,4S,5R,6R)-5-(2-acetoxyacetamido)-2-((1-amino-16-oxo-3,6,9,12-tetraoxa-15-azahenicosan-21-yl)oxy)-6-((1R,2R)-3-(2-(4-chlorophenyl)acetamido)-1,2-dihydroxypropyl)-4-hydroxytetrahydro-2H-pyran-2-carboxylate (3, 65.0 mg, 75.1 umol) in N,N-dimethylformamide (3.76 mL) were added N,N-dimethylaminopyridine (10.1 mg, 82.6 umol) and acetic anhydride (10.7 uL, 113 umol) at 0° C. and the reaction mixture was stirred at room temperature for 12 h. After completion, the reaction mixture was partitioned between ethyl acetate and water, organic layer was separated, dried over anhydrous sodium sulfate, and concentrated to afford the desired product (4) as a white sticky solid which was directly used for the next step without further purification. LCMS (ESI) m/z 907 [M+H]⁺.

### Synthesis of (2R,4S,5R,6R)-6-((1R,2R)-3-(2-(4-chlorophenyl)acetamido)-1,2-dihydroxypropyl)-2-((2,19-dioxo-6,9,12,15-tetraoxa-3,18-diazatetracosan-24-yl)oxy)-4-hydroxy-5-(2-hydroxyacetamido)tetrahydro-2H-pyran-2-carboxylic acid (Cpd. 228)

To a stirred solution of methyl (2R,4S,5R,6R)-5-(2-acetoxyacetamido)-6-((1R,2R)-3-(2-(4-chlorophenyl)acetamido)-1,2-dihydroxypropyl)-2-((2,19-dioxo-6,9,12,15-tetraoxa-3,18-diazatetracosan-24-yl)oxy)-4-hydroxytetrahydro-2H-pyran-2-carboxylate (4, 62.0 mg, 68.3 umol) in tetrahydrofuran (1.0 mL) was added a solution of lithium hydroxide monohydrate (8.18 mg, 342 umol) in water (0.1 mL). The reaction mixture was stirred at room temperature for 6 h. After completion, the reaction mixture was acidified with H⁺ exchange resin, filtered, concentrated, and purified by prep HPLC using water and acetonitrile as eluents to afford the desired product (Cpd. 228) as a white solid. Yield: 69 mg, 91%; LCMS (ESI) m/z 849.4 [M−H]⁻. ¹H NMR (500 MHz, DMSO-d₆): 7.99 (t, J=5.5 Hz, 1H), 7.88 (m, 1H), 7.84 (d, J=7.5 Hz, 1H), 7.82 (t, J=5.5 Hz, 1H), 7.35-7.31 (m, 2H), 7.27 (d, J=8.0 Hz, 2H), 3.88-3.86 (m, 2H), 3.66-3.60 (m, 4H), 3.55-3.49 (m, 16H), 3.37-3.27 (m, 4H), 3.21-3.15 (m, 8H), 2.05 (t, J=7.5 Hz, 2H), 3.39 (s, 3H), 1.54-1.51 (m, 1H), 1.49-1.42 (m, 4H), 1.25-1.19 (m, 2H).

### (2R,4S,5R,6R)-6-((1R,2R)-3-(2-(4-chlorophenyl)acetamido)-1,2-dihydroxypropyl)-2-((1-(2,5-dioxo-2,5-dihydro-1H-pyrrol-1-yl)-3,20-dioxo-7,10,13,16-tetraoxa-4,19-diazapentacosan-25-yl)oxy)-4-hydroxy-5-(2-hydroxyacetamido)tetrahydro-2H-pyran-2-carboxylic acid (Compound 228M)

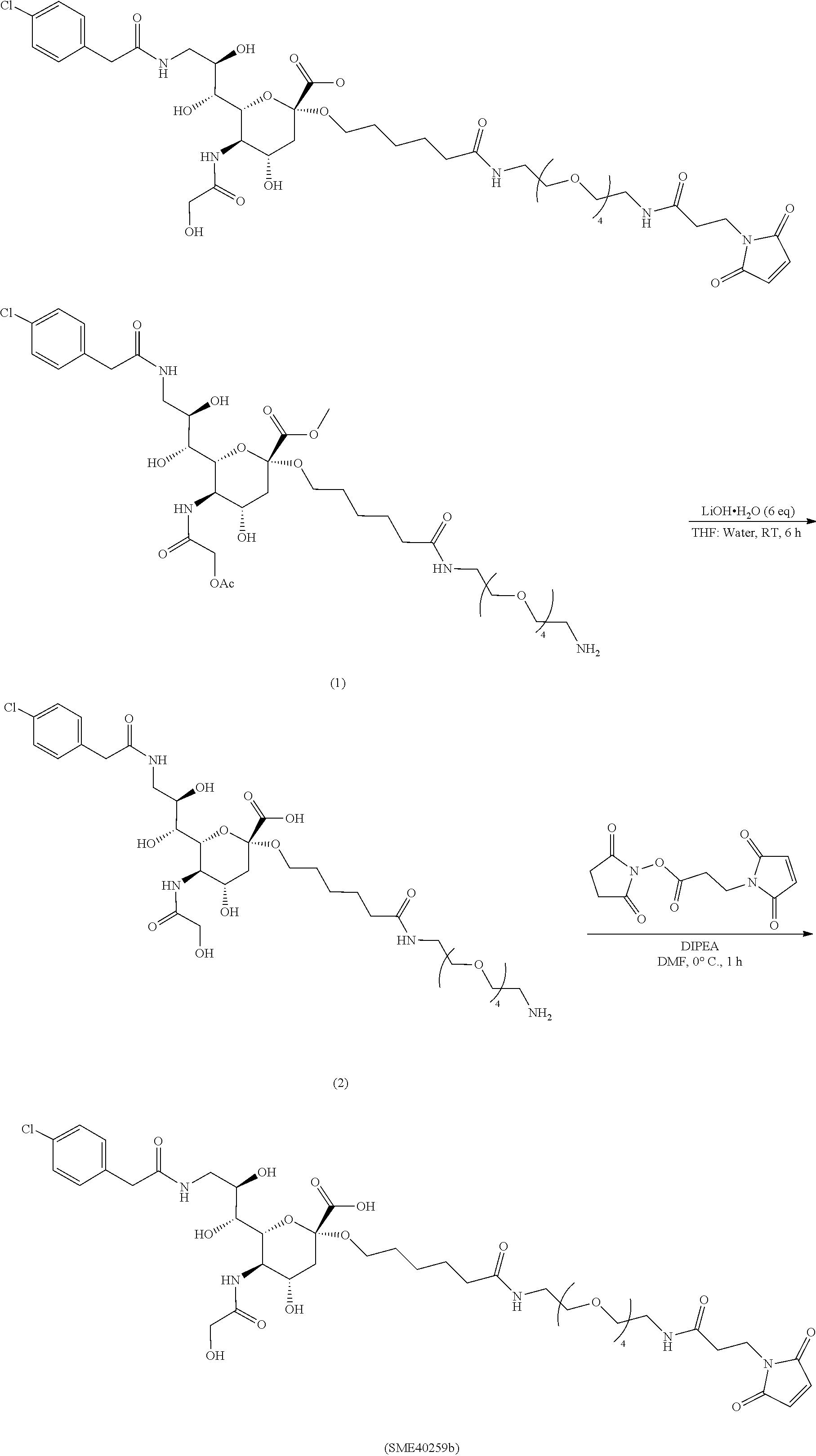

### Synthesis of (2R,4S,5R,6R)-2-((1-amino-16-oxo-3,6,9,12-tetraoxa-15-azahenicosan-21-yl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (2)

To a stirred solution of methyl (2R,4S,5R,6R)-5-(2-acetoxyacetamido)-2-((1-amino-16-oxo-3,6,9,12-tetraoxa-15-azahenicosan-21-yl)oxy)-6-((1R,2R)-3-(2-(4-chlorophenyl)acetamido)-1,2-dihydroxypropyl)-4-hydroxytetrahydro-2H-pyran-2-carboxylate (1, 76.2 mg, 88.1 umol) in tetrahydrofuran (3.2 mL) was added a solution of lithium hydroxide monohydrate (10.5 mg, 440 umol) in water (0.8 mL). The reaction mixture was stirred at room temperature for 6 h. After completion, the reaction mixture was acidified with H⁺ exchange resin, filtered, concentrated, and purified by prep HPLC using water and acetonitrile as eluents to afford the desired product (2) as a white solid. Yield: 62 mg, 87%; LCMS (ESI) m/z 809 [M+H]⁺.

### Synthesis of (2R,4S,5R,6R)-6-((1R,2R)-3-(2-(4-chlorophenyl)acetamido)-1,2-dihydroxypropyl)-2-((1-(2,5-dioxo-2,5-dihydro-1H-pyrrol-1-yl)-3,20-dioxo-7,10,13,16-tetraoxa-4,19-diazapentacosan-25-yl)oxy)-4-hydroxy-5-(2-hydroxyacetamido)tetrahydro-2H-pyran-2-carboxylic acid (Cpd. 228M)

To a stirred solution of (2R,4S,5R,6R)-2-((1-amino-16-oxo-3,6,9,12-tetraoxa-15-azahenicosan-21-yl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (2, 60.7 mg, 75.0 umol) in N,N-dimethylformamide (1.50 mL) were added 2,5-dioxopyrrolidin-1-yl 3-(2,5-dioxo-2,5-dihydro-1H-pyrrol-1-yl)propanoate (21.0 mg, 78.8 umol) and N,N-diisopropylethylamine (39.2 uL, 225 umol) at 0° C. and the reaction mixture was stirred at 0° C. for 1 h. After completion, the reaction mixture was filtered, concentrated, and purified by prep HPLC using water and acetonitrile as eluents to afford the desired product (Cpd. 228M) as a white solid. Yield: 72.0 mg, 72%; LCMS (ESI) m/z 860 [M+H]⁺.

### (2R,4S,5R,6R)-6-((1R,2R)-3-(2-chlorobenzamido)-1,2-dihydroxypropyl)-2-((6-((3,4-dioxo-2-((2-(2-(prop-2-yn-1-yloxy)ethoxy)ethyl)amino)cyclobut-1-en-1-yl)amino)hexyl)oxy)-4-hydroxy-5-(2-hydroxyacetamido)tetrahydro-2H-pyran-2-carboxylic acid (Compound 229)

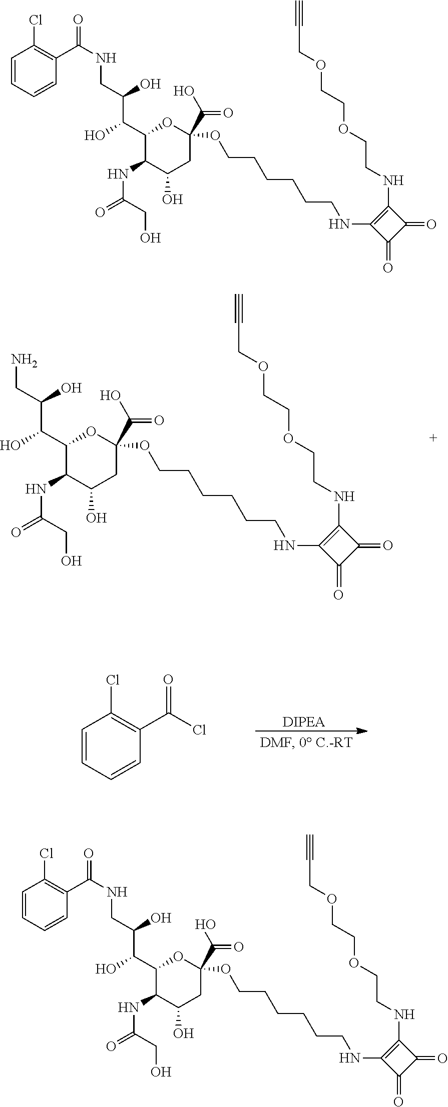 [SMILES: C#CCOCCOCCNc1c(NCCCCCCO[C@]2(C(=O)O)C[C@H](O)[C@@H](NC(=O)CO)[C@H]([C@H](O)[C@H](O)CNC(=O)c3ccccc3Cl)O2)c(=O)c1=O ; C#CCOCCOCCNc1c(NCCCCCCO[C@]2(C(=O)O)C[C@H](O)[C@@H](NC(=O)CO)[C@H]([C@H](O)[C@H](O)CN)O2)c(=O)c1=O ; "+" ; O=C(Cl)c1ccccc1Cl ; C#CCOCCOCCNc1c(NCCCCCCO[C@]2(C(=O)O)C[C@H](O)[C@@H](NC(=O)CO)[C@H]([C@H](O)[C@H](O)CNC(=O)c3ccccc3Cl)O2)c(=O)c1=O]

### Synthesis of (2R,4S,5R,6R)-6-((1R,2R)-3-(2-chlorobenzamido)-1,2-dihydroxypropyl)-2-((6-((3,4-dioxo-2-((2-(2-(prop-2-yn-1-yloxy)ethoxy)ethyl)amino)cyclobut-1-en-1-yl)amino)hexyl)oxy)-4-hydroxy-5-(2-hydroxyacetamido)tetrahydro-2H-pyran-2-carboxylic acid (Cpd. 229)

To a stirred solution of (2R,4S,5R,6R)-6-((1R,2R)-3-amino-1,2-dihydroxypropyl)-2-((6-((3,4-dioxo-2-((2-(2-(prop-2-yn-1-yloxy)ethoxy)ethyl)amino)cyclobut-1-en-1-yl)amino)hexyl)oxy)-4-hydroxy-5-(2-hydroxyacetamido)tetrahydro-2H-pyran-2-carboxylic acid (SME40289, 80.0 mg, 124 umol) in N,N-dimethylformamide (2.0 mL) was added 2-chlorobenzoyl chloride (16.5 uL, 130 umol) at room temperature. After 10 minutes, N,N-diisopropylethylamine (64.8 uL, 372 umol) was added dropwise at 0° C. The resulting reaction mixture was stirred at room temperature for 2 h. After completion, the reaction mixture was purified by prep HPLC using water and acetonitrile as eluents to afford the desired product (Cpd. 229) as a white solid. Yield: 56 mg, 56%; LCMS (ESI) m/z 781.6 [M−H]⁻. ¹H NMR (500 MHz, Methanol-d₄): 7.50-7.48 (m, 1H), 7.47-7.45 (m, 1H), 7.43-7.40 (m, 1H), 7.38-7.35 (m, 1H), 4.18 (d, J=2.5 Hz, 2H), 4.05 (s, 2H), 4.05-4.01 (m, 1H), 3.86-3.80 (m, 4H), 3.79-3.74 (m, 3H), 3.69-3.65 (m, 4H), 3.65-3.61 (m, 2H), 3.60 (m, 2H), 3.51-3.41 (m, 3H), 2.85 (t, J=2.5 Hz, 1H), 2.72 (dd, J=12.5 Hz & 4.0 Hz, 1H), 1.77-1.72 (t, J=12.5 Hz, 1H), 1.63-1.55 (m, 4H), 1.42-1.39 (m, 4H).

### (2R,4S,5R,6R)-6-((1R,2R)-3-benzamido-1,2-dihydroxypropyl)-2-((6-((3,4-dioxo-2-((2-(2-(prop-2-yn-1-yloxy)ethoxy)ethyl)amino)cyclobut-1-en-1-yl)amino)hexyl)oxy)-4-hydroxy-5-(2-hydroxyacetamido)tetrahydro-2H-pyran-2-carboxylic acid (Compound 230)

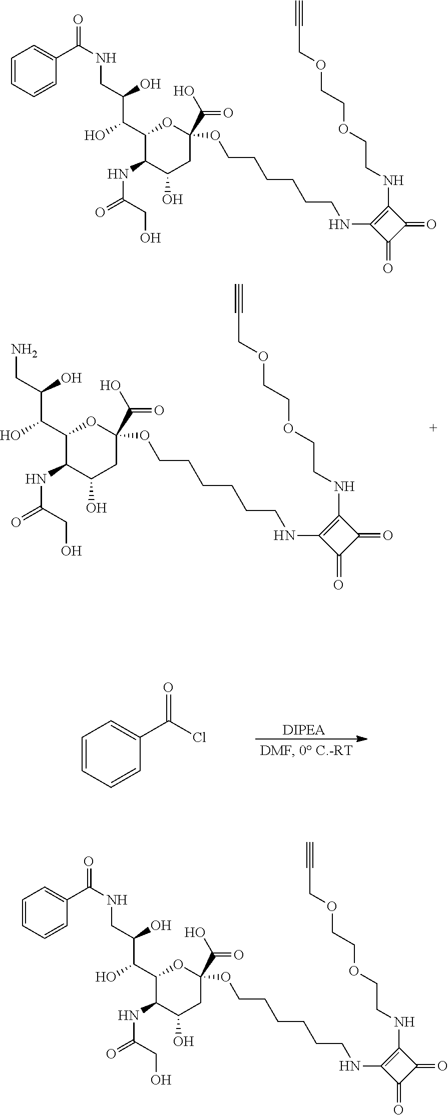 [SMILES: C#CCOCCOCCNc1c(NCCCCCCO[C@]2(C(=O)O)C[C@H](O)[C@@H](NC(=O)CO)[C@H]([C@H](O)[C@H](O)CNC(=O)c3ccccc3)O2)c(=O)c1=O ; C#CCOCCOCCNc1c(NCCCCCCO[C@]2(C(=O)O)C[C@H](O)[C@@H](NC(=O)CO)[C@H]([C@H](O)[C@H](O)CN)O2)c(=O)c1=O ; "+" ; O=C(Cl)c1ccccc1 ; C#CCOCCOCCNc1c(NCCCCCCO[C@]2(C(=O)O)C[C@H](O)[C@@H](NC(=O)CO)[C@H]([C@H](O)[C@H](O)CNC(=O)c3ccccc3)O2)c(=O)c1=O]

### Synthesis of (2R,4S,5R,6R)-6-((1R,2R)-3-benzamido-1,2-dihydroxypropyl)-2-((6-((3,4-dioxo-2-((2-(2-(prop-2-yn-1-yloxy)ethoxy)ethyl)amino)cyclobut-1-en-1-yl)amino)hexyl)oxy)-4-hydroxy-5-(2-hydroxyacetamido)tetrahydro-2H-pyran-2-carboxylic acid (Cpd. 230)

To a stirred solution of (2R,4S,5R,6R)-6-((1R,2R)-3-amino-1,2-dihydroxypropyl)-2-((6-((3,4-dioxo-2-((2-(2-(prop-2-yn-1-yloxy)ethoxy)ethyl)amino)cyclobut-1-en-1-yl)amino)hexyl)oxy)-4-hydroxy-5-(2-hydroxyacetamido)tetrahydro-2H-pyran-2-carboxylic acid (SME40289, 40.0 mg, 62.0 umol) in N,N-dimethylformamide (1.0 mL) was added benzoyl chloride (7.56 uL, 65.1 umol) at room temperature. After 10 minutes, N,N-diisopropylethylamine (32.4 uL, 186 umol) was added dropwise at 0° C. The resulting reaction mixture was stirred at room temperature for 2 h. After completion, the reaction mixture was purified by prep HPLC using water and acetonitrile as eluents to afford the desired product (Cpd. 230) as a white solid. Yield: 25 mg, 57%; LCMS (ESI) m/z 747.6 [M−H]⁻. ¹H NMR (500 MHz, Methanol-d₄): 7.83 (d, J=7.0 Hz, 2H), 7.53 (t, J=7.0 Hz, 1H), 7.46 (t, J=7.0 Hz, 2H), 4.17 (d, J=2.5 Hz, 2H), 4.04-4.00 (m, 1H), 4.02 (s, 2H), 3.85-3.83 (m, 2H), 3.82-3.74 (m, 4H), 3.69-3.65 (m, 4H), 3.64-3.62 (m, 2H), 3.58 (m, 2H), 3.54-3.40 (m, 4H), 2.85 (t, J=2.5 Hz, 1H), 2.71 (dd, J=12.5 Hz & 4.0 Hz, 1H), 1.77-1.72 (m, 1H), 1.61-1.55 (m, 4H), 1.41-1.38 (m, 4H).

### (2R,4S,5R,6R)-6-((1R,2R)-3-(4-(difluoromethyl)benzamido)-1,2-dihydroxypropyl)-2-((6-((3,4-dioxo-2-((2-(2-(prop-2-yn-1-yloxy)ethoxy)ethyl)amino)cyclobut-1-en-1-yl)amino)hexyl)oxy)-4-hydroxy-5-(2-hydroxyacetamido)tetrahydro-2H-pyran-2-carboxylic acid (Compound 231)

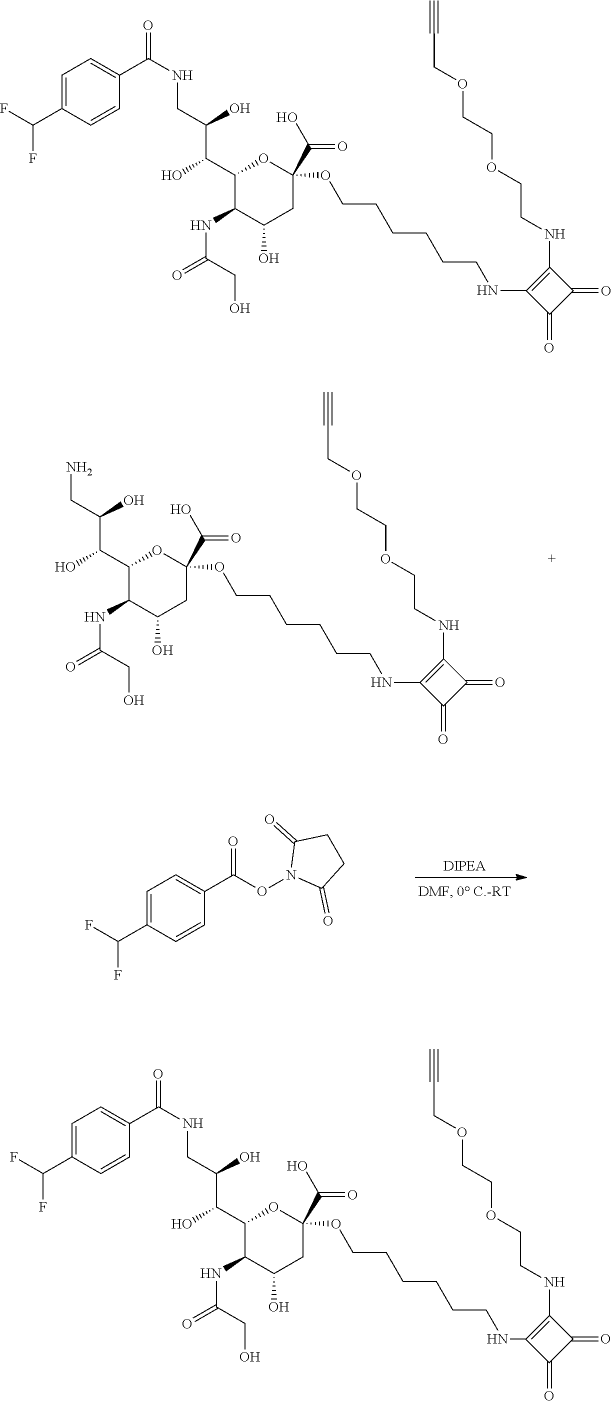 [SMILES: C#CCOCCOCCNc1c(NCCCCCCO[C@]2(C(=O)O)C[C@H](O)[C@@H](NC(=O)CO)[C@H]([C@H](O)[C@H](O)CNC(=O)c3ccc(C(F)F)cc3)O2)c(=O)c1=O ; C#CCOCCOCCNc1c(NCCCCCCO[C@]2(C(=O)O)C[C@H](O)[C@@H](NC(=O)CO)[C@H]([C@H](O)[C@H](O)CN)O2)c(=O)c1=O ; "+" ; O=C(ON1C(=O)CCC1=O)c1ccc(C(F)F)cc1 ; C#CCOCCOCCNc1c(NCCCCCCO[C@]2(C(=O)O)C[C@H](O)[C@@H](NC(=O)CO)[C@H]([C@H](O)[C@H](O)CNC(=O)c3ccc(C(F)F)cc3)O2)c(=O)c1=O]

### Synthesis of (2R,4S,5R,6R)-6-((1R,2R)-3-(4-(difluoromethyl)benzamido)-1,2-dihydroxypropyl)-2-((6-((3,4-dioxo-2-((2-(2-(prop-2-yn-1-yloxy)ethoxy)ethyl)amino)cyclobut-1-en-1-yl)amino)hexyl)oxy)-4-hydroxy-5-(2-hydroxyacetamido)tetrahydro-2H-pyran-2-carboxylic acid (Cpd. 231)

To a stirred solution of (2R,4S,5R,6R)-6-((1R,2R)-3-amino-1,2-dihydroxypropyl)-2-((6-((3,4-dioxo-2-((2-(2-(prop-2-yn-1-yloxy)ethoxy)ethyl)amino)cyclobut-1-en-1-yl)amino)hexyl)oxy)-4-hydroxy-5-(2-hydroxyacetamido)tetrahydro-2H-pyran-2-carboxylic acid (SME40289, 40.0 mg, 62.0 umol) in N,N-dimethylformamide (1.0 mL) was added 2,5-dioxopyrrolidin-1-yl 4-(difluoromethyl)benzoate (15.5 mg, 65.1 umol) at room temperature. After 10 minutes, N,N-diisopropylethylamine (32.4 uL, 186 umol) was added dropwise at 0° C. The resulting reaction mixture was stirred at room temperature for 2 h. After completion, the reaction mixture was purified by prep HPLC using water and acetonitrile as eluents to afford the desired product (Cpd. 231) as a white solid. Yield: 29 mg, 59%; LCMS (ESI) m/z 797.7 [M−H]⁻. ¹H NMR (500 MHz, Methanol-d₄): 7.94 (d, J=8.0 Hz, 2H), 7.64 (d, J=8.0 Hz, 2H), 6.83 (t, J=56.0 Hz, 1H), 4.17 (d, J=2.5 Hz, 2H), 4.05-4.01 (m, 1H), 4.02 (s, 2H), 3.85-3.82 (m, 3H), 3.81-3.75 (m, 4H), 3.68-3.65 (m, 4H), 3.64-3.62 (m, 2H), 3.59 (m, 2H), 3.54-3.40 (m, 3H), 2.85 (t, J=2.5 Hz, 1H), 2.71 (dd, J=12.5 Hz & 4.0 Hz, 1H), 1.77-1.72 (m, 1H), 1.61-1.55 (m, 4H), 1.42-1.39 (m, 4H).

### (2R,4S,5R,6R)-2-((6-((3,4-dioxo-2-((2-(2-(prop-2-yn-1-yloxy)ethoxy)ethyl)amino)cyclobut-1-en-1-yl)amino)hexyl)oxy)-6-((1R,2R)-3-(4-fluorobenzamido)-1,2-dihydroxypropyl)-4-hydroxy-5-(2-hydroxyacetamido)tetrahydro-2H-pyran-2-carboxylic acid (Compound 232)

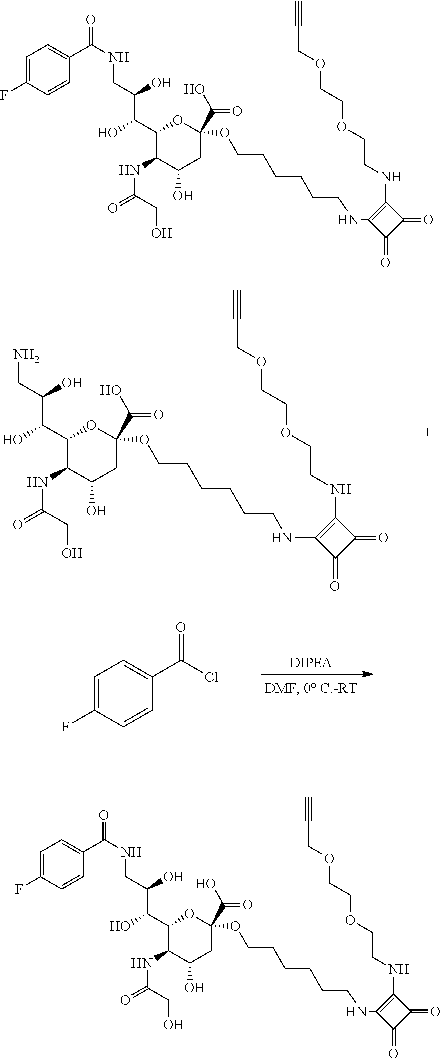 [SMILES: C#CCOCCOCCNc1c(NCCCCCCO[C@]2(C(=O)O)C[C@H](O)[C@@H](NC(=O)CO)[C@H]([C@H](O)[C@H](O)CNC(=O)c3ccc(F)cc3)O2)c(=O)c1=O ; C#CCOCCOCCNc1c(NCCCCCCO[C@]2(C(=O)O)C[C@H](O)[C@@H](NC(=O)CO)[C@H]([C@H](O)[C@H](O)CN)O2)c(=O)c1=O ; "+" ; O=C(Cl)c1ccc(F)cc1 ; C#CCOCCOCCNc1c(NCCCCCCO[C@]2(C(=O)O)C[C@H](O)[C@@H](NC(=O)CO)[C@H]([C@H](O)[C@H](O)CNC(=O)c3ccc(F)cc3)O2)c(=O)c1=O]

### Synthesis of (2R,4S,5R,6R)-2-((6-((3,4-dioxo-2-((2-(2-(prop-2-yn-1-yloxy)ethoxy)ethyl) amino)cyclobut-1-en-1-yl)amino)hexyl)oxy)-6-((1R,2R)-3-(4-fluorobenzamido)-1,2-dihydroxypropyl)-4-hydroxy-5-(2-hydroxyacetamido)tetrahydro-2H-pyran-2-carboxylic acid (Cpd. 232)

To a stirred solution of (2R,4S,5R,6R)-6-((1R,2R)-3-amino-1,2-dihydroxypropyl)-2-((6-((3,4-dioxo-2-((2-(2-(prop-2-yn-1-yloxy)ethoxy)ethyl)amino)cyclobut-1-en-1-yl)amino)hexyl)oxy)-4-hydroxy-5-(2-hydroxyacetamido)tetrahydro-2H-pyran-2-carboxylic acid (SME40289, 40.0 mg, 62.0 umol) in N,N-dimethylformamide (1.0 mL) was added 4-fluorobenzoyl chloride (8.53 uL, 65.1 umol) at room temperature. After 10 minutes, N,N-diisopropylethylamine (32.4 uL, 186 umol) was added dropwise at 0° C. The resulting reaction mixture was stirred at room temperature for 2 h. After completion, the reaction mixture was purified by prep HPLC using water and acetonitrile as eluents to afford the desired product (Cpd. 232) as a white solid. Yield: 28 mg, 59%; LCMS (ESI) m/z 765.5 [M−H]⁻. ¹H NMR (500 MHz, Methanol-d₄): 7.90-7.87 (m, 2H), 7.18 (t, J=8.0 Hz, 2H), 4.17 (d, J=2.5 Hz, 2H), 4.03-4.00 (m, 1H), 4.02 (s, 2H), 3.85-3.83 (m, 2H), 3.81-3.74 (m, 4H), 3.69-3.62 (m, 6H), 3.59 (m, 2H), 3.52-3.40 (m, 4H), 2.85 (t, J=2.5 Hz, 1H), 2.71 (dd, J=12.5 Hz & 3.5 Hz, 1H), 1.76-1.71 (m, 1H), 1.61-1.55 (m, 4H), 1.42-1.39 (m, 4H).

### (2R,4S,5R,6R)-6-((1R,2R)-3-(3-(difluoromethyl)benzamido)-1,2-dihydroxypropyl)-2-((6-((3,4-dioxo-2-((2-(2-(prop-2-yn-1-yloxy)ethoxy)ethyl)amino)cyclobut-1-en-1-yl)amino)hexyl)oxy)-4-hydroxy-5-(2-hydroxyacetamido)tetrahydro-2H-pyran-2-carboxylic acid (Compound 233)

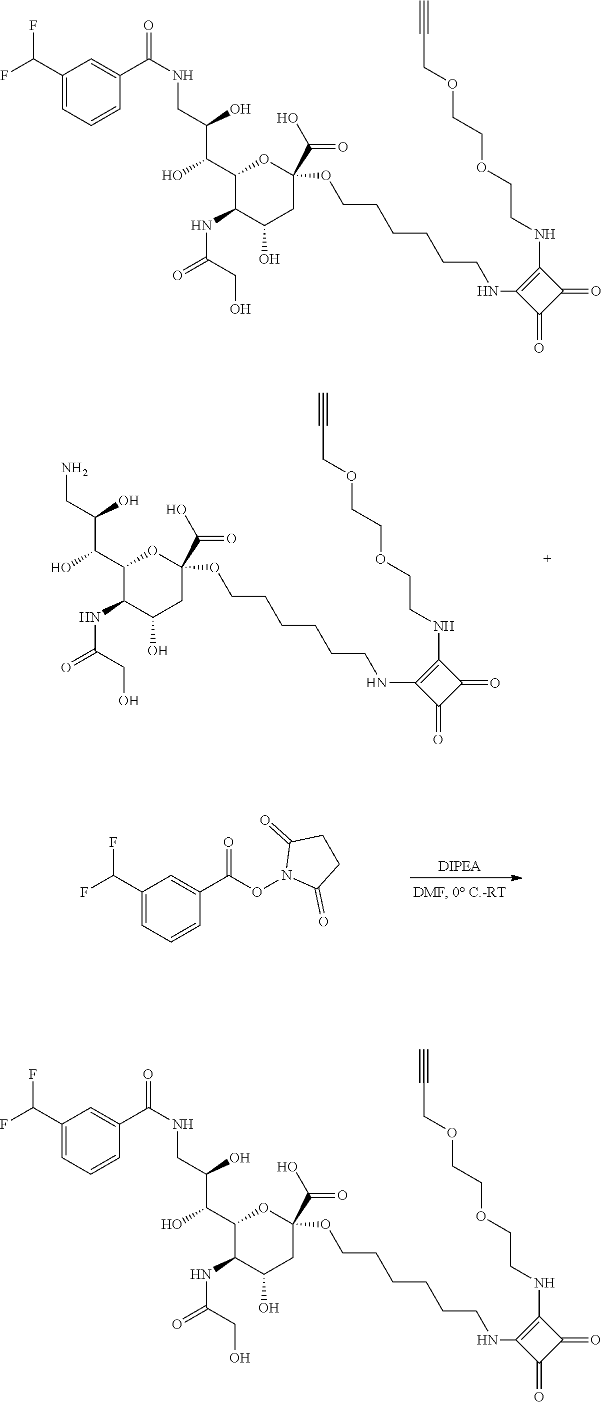 [SMILES: C#CCOCCOCCNc1c(NCCCCCCO[C@]2(C(=O)O)C[C@H](O)[C@@H](NC(=O)CO)[C@H]([C@H](O)[C@H](O)CNC(=O)c3cccc(C(F)F)c3)O2)c(=O)c1=O ; C#CCOCCOCCNc1c(NCCCCCCO[C@]2(C(=O)O)C[C@H](O)[C@@H](NC(=O)CO)[C@H]([C@H](O)[C@H](O)CN)O2)c(=O)c1=O ; "+" ; O=C(ON1C(=O)CCC1=O)c1cccc(C(F)F)c1 ; C#CCOCCOCCNc1c(NCCCCCCO[C@]2(C(=O)O)C[C@H](O)[C@@H](NC(=O)CO)[C@H]([C@H](O)[C@H](O)CNC(=O)c3cccc(C(F)F)c3)O2)c(=O)c1=O]

### Synthesis of (2R,4S,5R,6R)-6-((1R,2R)-3-(3-(difluoromethyl)benzamido)-1,2-dihydroxypropyl)-2-((6-((3,4-dioxo-2-((2-(2-(prop-2-yn-1-yloxy)ethoxy)ethyl)amino)cyclobut-1-en-1-yl)amino)hexyl)oxy)-4-hydroxy-5-(2-hydroxyacetamido)tetrahydro-2H-pyran-2-carboxylic acid (Cpd. 233)

To a stirred solution of (2R,4S,5R,6R)-6-((1R,2R)-3-amino-1,2-dihydroxypropyl)-2-((6-((3,4-dioxo-2-((2-(2-(prop-2-yn-1-yloxy)ethoxy)ethyl)amino)cyclobut-1-en-1-yl)amino)hexyl)oxy)-4-hydroxy-5-(2-hydroxyacetamido)tetrahydro-2H-pyran-2-carboxylic acid (SME40289, 40.0 mg, 62.0 umol) in N,N-dimethylformamide (1.0 mL) was added 2,5-dioxopyrrolidin-1-yl 3-(difluoromethyl)benzoate (17.5 mg, 65.1 umol) at room temperature. After 10 minutes, N,N-diisopropylethylamine (32.4 uL, 186 umol) was added dropwise at 0° C. The resulting reaction mixture was stirred at room temperature for 2 h. After completion, the reaction mixture was purified by prep HPLC using water and acetonitrile as eluents to afford the desired product (Cpd. 233) as a white solid. Yield: 22.8 mg, 47%; LCMS (ESI) m/z 797.6 [M−H]⁻. ¹H NMR (500 MHz, Methanol-d₄): 8.02 (s, 1H), 7.98 (d, J=8.0 Hz, 1H), 7.71 (d, J=8.0 Hz, 2H), 7.59 (t, J=8.0 Hz, 1H), 6.84 (t, J=56.0 Hz, 1H), 4.17 (d, J=2.5 Hz, 2H), 4.06-4.02 (m, 1H), 4.02 (s, 2H), 3.86-3.82 (m, 3H), 3.81-3.75 (m, 4H), 3.69-3.65 (m, 4H), 3.64-3.62 (m, 2H), 3.58 (m, 2H), 3.55-3.40 (m, 3H), 2.85 (t, J=2.5 Hz, 1H), 2.71 (dd, J=12.5 Hz & 4.0 Hz, 1H), 1.77-1.72 (m, 1H), 1.61-1.55 (m, 4H), 1.42-1.38 (m, 4H).

### (2R,4S,5R,6R)-2-((6-((3,4-dioxo-2-((2-(2-(prop-2-yn-1-yloxy)ethoxy)ethyl)amino)cyclobut-1-en-1-yl)amino)hexyl)oxy)-6-((1R,2R)-3-(3-fluorobenzamido)-1,2-dihydroxypropyl)-4-hydroxy-5-(2-hydroxyacetamido)tetrahydro-2H-pyran-2-carboxylic acid (Compound 234)

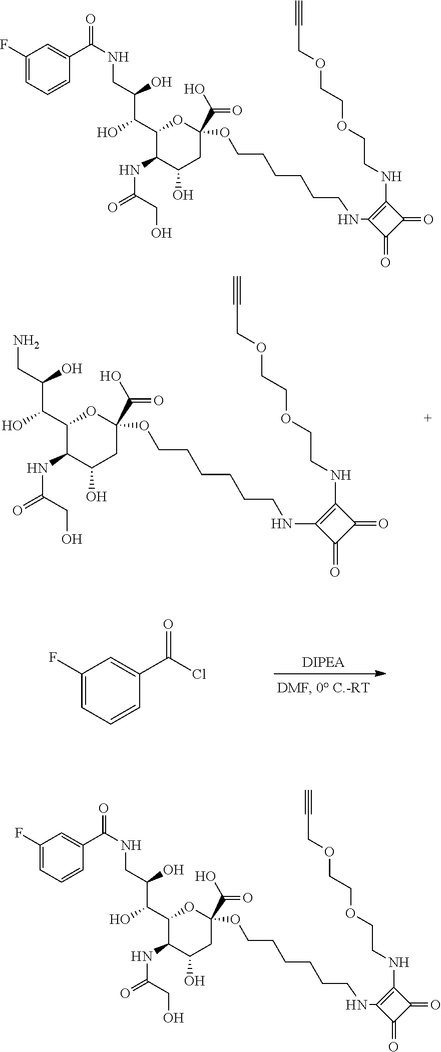 [SMILES: C#CCOCCOCCNc1c(NCCCCCCO[C@]2(C(=O)O)C[C@H](O)[C@@H](NC(=O)CO)[C@H]([C@H](O)[C@H](O)CNC(=O)c3cccc(F)c3)O2)c(=O)c1=O ; C#CCOCCOCCNc1c(NCCCCCCO[C@]2(C(=O)O)C[C@H](O)[C@@H](NC(=O)CO)[C@H]([C@H](O)[C@H](O)CN)O2)c(=O)c1=O ; "+" ; O=C(Cl)c1cccc(F)c1 ; C#CCOCCOCCNc1c(NCCCCCCO[C@]2(C(=O)O)C[C@H](O)[C@@H](NC(=O)CO)[C@H]([C@H](O)[C@H](O)CNC(=O)c3cccc(F)c3)O2)c(=O)c1=O]

### Synthesis of (2R,4S,5R,6R)-2-((6-((3,4-dioxo-2-((2-(2-(prop-2-yn-1-yloxy)ethoxy)ethyl)amino)cyclobut-1-en-1-yl)amino)hexyl)oxy)-6-((1R,2R)-3-(3-fluorobenzamido)-1,2-dihydroxypropyl)-4-hydroxy-5-(2-hydroxyacetamido)tetrahydro-2H-pyran-2-carboxylic acid (Cpd. 234)

To a stirred solution of (2R,4S,5R,6R)-6-((1R,2R)-3-amino-1,2-dihydroxypropyl)-2-((6-((3,4-dioxo-2-((2-(2-(prop-2-yn-1-yloxy)ethoxy)ethyl)amino)cyclobut-1-en-1-yl)amino)hexyl)oxy)-4-hydroxy-5-(2-hydroxyacetamido)tetrahydro-2H-pyran-2-carboxylic acid (SME40289, 40.0 mg, 62.0 umol) in N,N-dimethylformamide (1.0 mL) was added 3-fluorobenzoyl chloride (7.92 uL, 65.1 umol) at room temperature. After 10 minutes, N,N-diisopropylethylamine (32.4 uL, 186 umol) was added dropwise at 0° C. The resulting reaction mixture was stirred at room temperature for 2 h. After completion, the reaction mixture was purified by prep HPLC using water and acetonitrile as eluents to afford the desired product (Cpd. 234) as a white solid. Yield: 23 mg, 50%; LCMS (ESI) m/z 765.5 [M−H]⁻. ¹H NMR (500 MHz, Methanol-d₄): 7.66 (d, J=8.0 Hz, 1H), 7.58-7.56 (m, 1H), 7.50-7.46 (m, 1H), 7.29-7.25 (m, 1H), 4.17 (d, J=2.5 Hz, 2H), 4.04-4.00 (m, 1H), 4.02 (s, 2H), 3.85-3.82 (m, 2H), 3.81-3.75 (m, 4H), 3.69-3.62 (m, 6H), 3.59 (m, 2H), 3.53-3.40 (m, 4H), 2.85 (t, J=2.5 Hz, 1H), 2.71 (dd, J=12.5 Hz & 3.5 Hz, 1H), 1.76-1.71 (m, 1H), 1.61-1.55 (m, 4H), 1.42-1.39 (m, 4H).

### (2R,4S,5R,6R)-2-((6-((2-((14-carboxy-3,6,9,12-tetra oxa-tetradecyl)amino)-3,4-dioxocyclobut-1-en-1-yl)amino)hexyl)oxy)-6-((1R,2R)-3-(2-(4-chlorophenyl) acetamido)-1,2-dihydroxypropyl)-4-hydroxy-5-(2-hydroxyacetamido) tetrahydro-2H-pyran-2-carboxylic acid (Compound 154)

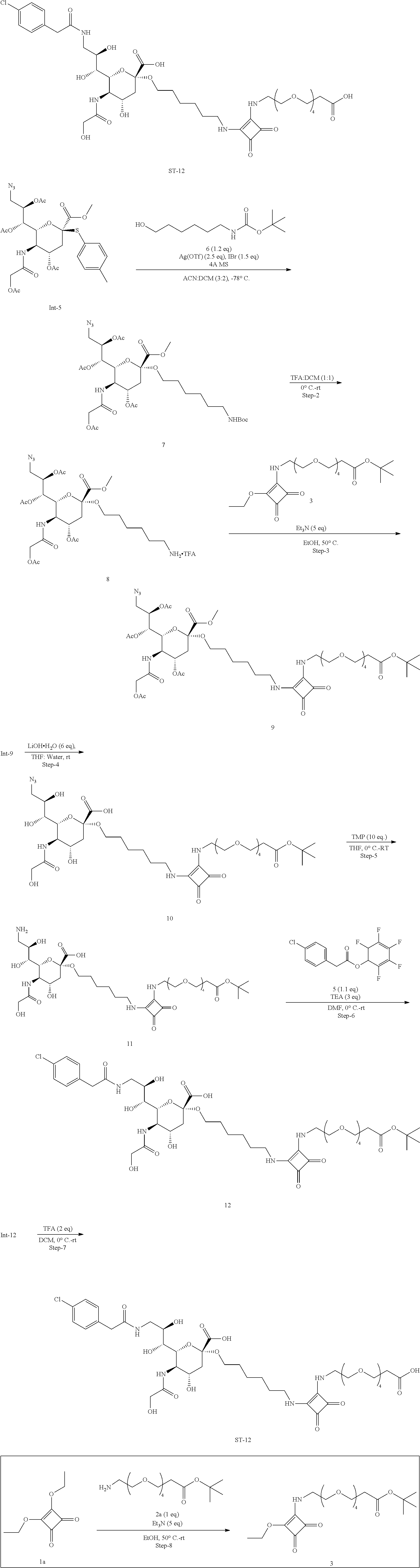

### Synthesis of (1R,2R)-1-((2R,3R,4S,6R)-4-acetoxy-3-(2-acetoxyacetamido)-6-((6-((tert-butoxycarbonyl)amino)hexyl)oxy)-6-(methoxy carbonyl) tetrahydro-2H-pyran-2-yl)-3-azidopropane-1,2-diyl diacetate (7)

A solution (1R,2R)-1-((2R,3R,4S,6R)-4-acetoxy-3-(2-acetoxyacetamido)-6-(methoxycarbonyl)-6-(p-tolylthio) tetrahydro-2H-pyran-2-yl)-3-azidopropane-1,2-diyl diacetate (Int-5, 2 g, 0.26 mmol) and tert-butyl (6-hydroxyhexyl)carbamate (2 g, 3.13 mmol), Silver (1+) trifluoro methane sulfonate (2 g, 9.39 mmol) and activated 4 Å powdered molecular sieves (5 g) in anhydrous dichloromethane (12 mL) and anhydrous acetonitrile (24 mL) was stirred at room temperature for 1 h under nitrogen atmosphere. The solution was cooled to −78° C. and a solution of iodine monobromide (1.94 g, 9.39 mmol) in dichloromethane (4 mL) was added dropwise to the reaction mixture and was stirred at same temperature for 2 h. After completion, the reaction mixture was quenched by addition of triethylamine (5 mL) and warmed to room temperature. The reaction mixture was filtered. The filtrate was washed with saturated aqueous solution of sodium thiosulfate and dried over sodium sulfate, filtered and concentrated. The crude product was purified by silica gel column chromatography using ethyl acetate and hexane as eluents to afford the desired product (1R,2R)-1-((2R,3R,4S,6R)-4-acetoxy-3-(2-acetoxyacetamido)-6-((6-((tert-butoxycarbonyl)amino)hexyl)oxy)-6-(methoxy carbonyl) tetrahydro-2H-pyran-2-yl)-3-azidopropane-1,2-diyl diacetate (7) as a colorless sticky liquid. Yield: 1.3 g, 56%; LCMS m/z 732.7 [M+H]⁺.

### Synthesis of (1R,2R)-1-((2R,3R,4S,6R)-4-acetoxy-3-(2-acetoxyacetamido)-6-((6-aminohexyl) oxy)-6-(methoxy carbonyl) tetrahydro-2H-pyran-2-yl)-3-azidopropane-1,2-diyl diacetate (8)

To a stirred solution of (1R,2R)-1-((2R,3R,4S,6R)-4-acetoxy-3-(2-acetoxyacetamido)-6-((6-((tert-butoxycarbonyl)amino)hexyl)oxy)-6-(methoxy carbonyl) tetrahydro-2H-pyran-2-yl)-3-azidopropane-1,2-diyl diacetate 7 (1.2 g, 1.64 mmol) in anhydrous dichloromethane (10 mL), was added trifluoro acetic acid (3.0 mL) at 0° C. The reaction mixture was stirred at room temperature for 3 h. After completion, solvent was concentrated under reduced pressure to afford the desired product (1R,2R)-1-((2R,3R,4S,6R)-4-acetoxy-3-(2-acetoxyacetamido)-6-((6-aminohexyl)oxy)-6-(methoxycarbonyl) tetrahydro-2H-pyran-2-yl)-3-azidopropane-1,2-diyl diacetate (8) as a light yellow sticky syrup. The crude residue was directly used for the next step without further purification. Yield: 1.2 g, Crude; LCMS (ESI+) m/z 632.4 [M+H]⁺.

### Synthesis of (1R,2R)-1-((2R,3R,4S,6R)-4-acetoxy-3-(2-acetoxyacetamido)-6-((6-((2-((17,17-dimethyl-15-oxo-3,6,9,12,16-pentaoxaoctadecyl)amino)-3,4-dioxocyclobut-1-en-1-yl)amino) hexyl)oxy)-6-(methoxy carbonyl) tetrahydro-2H-pyran-2-yl)-3-azidopropane-1,2-diyl diacetate (9)

To a stirred solution of (1R,2R)-1-((2R,3R,4S,6R)-4-acetoxy-3-(2-acetoxyacetamido)-6-((6-aminohexyl)oxy)-6-(methoxycarbonyl)tetrahydro-2H-pyran-2-yl)-3-azidopropane-1,2-diyl diacetate 8 (1.2 g, 1.89 mmol) in ethanol (5 mL), was added triethylamine (0.799 mL, 5.69 mmol). After 10 minutes 3-ethoxy-4-[(prop-2-yn-1-yl)amino] cyclobut-3-ene-1,2-dione (3, 1.01 g, 2.27 mmol) was added at room temperature, and the resulting reaction mixture was stirred at 50° C. for 3 h. After completion, the reaction mixture was concentrated under reduced pressure and the crude residue obtained was purified by silica gel column chromatography using methanol in dichloromethane as eluents to afford the desired product (1R,2R)-1-((2R,3R,4S,6R)-4-acetoxy-3-(2-acetoxyacetamido)-6-((6-((2-((17,17-dimethyl-15-oxo-3,6,9,12,16-pentaoxaoctadecyl)amino)-3,4-dioxocyclobut-1-en-1-yl)amino)hexyl)oxy)-6-(methoxycarbonyl)tetrahydro-2H-pyran-2-yl)-3-azidopropane-1,2-diyl diacetate (9) as yellow solid. Yield: 700 mg, 36%; LCMS (ESI+) m/z 1031.8 [M+H]⁺.

### Synthesis of (2R,4S,5R,6R)-6-((1R,2R)-3-azido-1,2-dihydroxypropyl)-2-((6-((2-((17,17-dimethyl-15-oxo-3,6,9,12,16-penta oxa-octadecyl)amino)-3,4-dioxocyclobut-1-en-1-yl)amino)hexyl) oxy)-4-hydroxy-5-(2-hydroxy acetamido) tetrahydro-2H-pyran-2-carboxylic acid (10)

To a stirred solution of (1R,2R)-1-((2R,3R,4S,6R)-4-acetoxy-3-(2-acetoxyacetamido)-6-((6-((2-((17,17-dimethyl-15-oxo-3,6,9,12,16-penta oxa-octadecyl)amino)-3,4-dioxocyclobut-1-en-1-yl)amino)hexyl)oxy)-6-(methoxy carbonyl) tetrahydro-2H-pyran-2-yl)-3-azidopropane-1,2-diyl diacetate 9 (700 mg, 0.67 mmol) in Tetrahydrofuran (5 mL) was added solution of lithium hydroxide monohydrate (278 mg, 6.78 mmol) in water (2 mL). The reaction mixture was stirred at room temperature for 6 h. After completion, the reaction mixture was treated with acidic resin (Dowex 50, H⁺) to pH˜6 and the suspension was filtered. The filtrate was concentrated to afford the desired crude product ((2R,4S,5R,6R)-6-((1R,2R)-3-azido-1,2-dihydroxypropyl)-2-((6-((2-((17,17-dimethyl-15-oxo-3,6,9,12,16-pentaoxaoctadecyl)amino)-3,4-dioxocyclobut-1-en-1-yl) amino)hexyl)oxy)-4-hydroxy-5-(2-hydroxyacetamido) tetrahydro-2H-pyran-2-carboxylic acid (10) which was used without further purification. Yield: 600 mg, Crude. LCMS (ESI+) m/z 849.8 [M+H]⁺.

### Synthesis of (2R,4S,5R,6R)-6-((1R,2R)-3-amino-1,2-dihydroxypropyl)-2-((6-((2-((17,17-dimethyl-15-oxo-3,6,9,12,16-penta oxa-octadecyl)amino)-3,4-dioxocyclobut-1-en-1-yl)amino) hexyl)oxy)-4-hydroxy-5-(2-hydroxyacetamido) tetrahydro-2H-pyran-2-carboxylic acid (11)

To a stirred solution of (2R,4S,5R,6R)-6-((1R,2R)-3-azido-1,2-dihydroxypropyl)-2-((6-((2-((17,17-dimethyl-15-oxo-3,6,9,12,16-penta oxa-octadecyl)amino)-3,4-dioxocyclobut-1-en-1-yl)amino)hexyl)oxy)-4-hydroxy-5-(2-hydroxy acetamido) tetrahydro-2H-pyran-2-carboxylic acid (10) (600 mg, 0.70 mmol) in water (6 mL) and NH₄OH (0.1 mL, pH>8) was added trimethyl phosphine (1.06 mL, 1.5 eq, 1M soln in THF) at 0° C. and the reaction mixture was stirred for 1 h at the same temperature. After completion, the reaction mixture was washed with ethyl acetate, and the aqueous layer was dried by lyophilization to afford the desired crude product (2R,4S,5R,6R)-6-((1R,2R)-3-amino-1,2-dihydroxypropyl)-2-((6-((2-((17,17-dimethyl-15-oxo-3,6,9,12,16-pentaoxaoctadecyl)amino)-3,4-dioxocyclobut-1-en-1-yl)amino)hexyl)oxy)-4-hydroxy-5-(2-hydroxyacetamido) tetrahydro-2H-pyran-2-carboxylic acid (11) as a white solid which was used without further purification. Yield: 250 mg, Crude. LCMS (ESI+) m/z 823.7 [M+H]⁺.

### Synthesis of (2R,4S,5R,6R)-6-((1R,2R)-3-(2-(4-chlorophenyl) acetamido)-1,2-dihydroxypropyl)-2-((6-((2-((17,17-dimethyl-15-oxo-3,6,9,12,16-penta oxa-octadecyl)amino)-3,4-dioxocyclobut-1-en-1-yl)amino)hexyl)oxy)-4-hydroxy-5-(2-hydroxy acetamido) tetrahydro-2H-pyran-2-carboxylic acid (12)

To a stirred solution of (2R,4S,5R,6R)-6-((1R,2R)-3-amino-1,2-dihydroxypropyl)-2-((6-((2-((17,17-dimethyl-15-oxo-3,6,9,12,16-pentaoxaoctadecyl)amino)-3,4-dioxocyclobut-1-en-1-yl)amino)hexyl)oxy)-4-hydroxy-5-(2-hydroxyacetamido)tetrahydro-2H-pyran-2-carboxylic acid (11) (250 mg, 0.30 mmol) in DMF (2 mL) was added perfluoro phenyl 2-(4-chlorophenyl)acetate (110 mg, 0.33 mmol, 1.1 eq) at room temperature. After 10 minutes, triethylamine (0.12 mL, 0.91 mmol) was added dropwise at 0° C. The resulting reaction mixture was stirred at rt for 2 h. After completion the reaction mixture was purified by C18 reverse phase column chromatography using acetonitrile and water (+0.05% TFA) to afford the desired product (2R,4S,5R,6R)-6-((1R,2R)-3-(2-(4-chlorophenyl) acetamido)-1,2-dihydroxypropyl)-2-((6-((2-((17,17-dimethyl-15-oxo-3,6,9,12,16-pentaoxaoctadecyl)amino)-3,4-dioxocyclobut-1-en-1-yl) amino)hexyl)oxy)-4-hydroxy-5-(2-hydroxyacetamido) tetrahydro-2H-pyran-2-carboxylic acid (12) as white solid, Yield: 100 mg, 34%. LCMS (ESI+) m/z 975.8 [M+H]⁺

### Synthesis of (2R,4S,5R,6R)-2-((6-((2-((14-carboxy-3,6,9,12-tetra oxa-tetra decyl)amino)-3,4-dioxocyclobut-1-en-1-yl)amino)hexyl)oxy)-6-((1R,2R)-3-(2-(4-chlorophenyl) acetamido)-1,2-dihydroxypropyl)-4-hydroxy-5-(2-hydroxyacetamido) tetrahydro-2H-pyran-2-carboxylic acid (154)

To a stirred solution of (2R,4S,5R,6R)-6-((1R,2R)-3-(2-(4-chlorophenyl) acetamido)-1,2-dihydroxypropyl)-2-((6-((2-((17,17-dimethyl-15-oxo-3,6,9,12,16-pentaoxaoctadecyl) amino)-3,4-dioxocyclobut-1-en-1-yl)amino)hexyl)oxy)-4-hydroxy-5-(2-hydroxyacetamido) tetrahydro-2H-pyran-2-carboxylic acid (12, 100 mg, 0.10 mmol) in anhydrous dichloromethane (1 mL) was added trifluoroacetic acid (1 mL) dropwise at 0° C. The reaction mixture was stirred at room temperature for 3 h. After completion, the reaction mixture was directly purified by prep HPLC using acetonitrile and water (+0.05% TFA) as eluents to afford the desired product (2R,4S,5R,6R)-2-((6-((2-((14-carboxy-3,6,9,12-tetra oxa-tetra decyl)amino)-3,4-dioxocyclobut-1-en-1-yl)amino)hexyl)oxy)-6-((1R,2R)-3-(2-(4-chlorophenyl) acetamido)-1,2-dihydroxypropyl)-4-hydroxy-5-(2-hydroxyacetamido) tetrahydro-2H-pyran-2-carboxylic acid (12) as off white solid. Yield: 87 mg, 95%. LCMS (ESI+) m/z 919.8 [M+H]⁺. ¹H NMR (401 MHz, MeOD) δ 7.31 (s, 4H), 4.04 (s, 2H), 3.93-3.79 (m, 6H), 3.76 (d, J=6.3 Hz, 3H), 3.73 (d, J=2.8 Hz, 2H), 3.66 (s, 6H), 3.65 (s, 6H), 3.63 (d, J=1.6 Hz, 4H), 3.56 (s, 2H), 3.41-3.37 (m, 2H), 3.23 (d, J=5.6 Hz, 1H), 2.74 (dd, J=12.7, 4.1 Hz, 1H), 2.56 (s, 1H), 1.73 (t, J=11.9 Hz, 1H), 1.64-1.56 (m, 4H), 1.41 (s, 4H), 1.31 (s, 1H).

### Synthesis of tert-butyl 1-((2-ethoxy-3,4-dioxocyclobut-1-en-1-yl)amino)-3,6,9,12-tetraoxapentadecan-15-oate (3a)

To a stirred solution of 3,4-diethoxycyclobut-3-ene-1,2-dione (1 g, 5.88 mmol) in ethanol (10 mL) was added tert-butyl 1-amino-3,6,9,12-tetraoxapentadecan-15-oate (1.8 g, 5.88 mmol) at room temperature. After 10 minutes, triethylamine (4.1 mL, 29.4 mmol) was added dropwise at 0° C. The resulting reaction mixture was stirred at room temperature for 6 h. After completion the reaction mixture was concentrated to afford the desired crude product tert-butyl 1-((2-ethoxy-3,4-dioxocyclobut-1-en-1-yl)amino)-3,6,9,12-tetraoxapentadecan-15-oate (3a) as a white solid. The crude was directly used for the next step without further purification. Yield: 1.5 g, 57%. LCMS (ESI−) m/z 444.3 [M−H]−.

### (2R,4S,5R,6R)-6-((1R,2R)-1,2-dihydroxy-3-(3-phenoxybenzamido) propyl)-2-((6-((3,4-dioxo-2-((2-(2-(prop-2-yn-1-yloxy) ethoxy)ethyl)amino) cyclobut-1-en-1-yl)amino)hexyl) oxy) -4-hydroxy-5-(2-hydroxyacetamido) tetrahydro-2H-pyran-2-carboxylic acid (Compound 39)

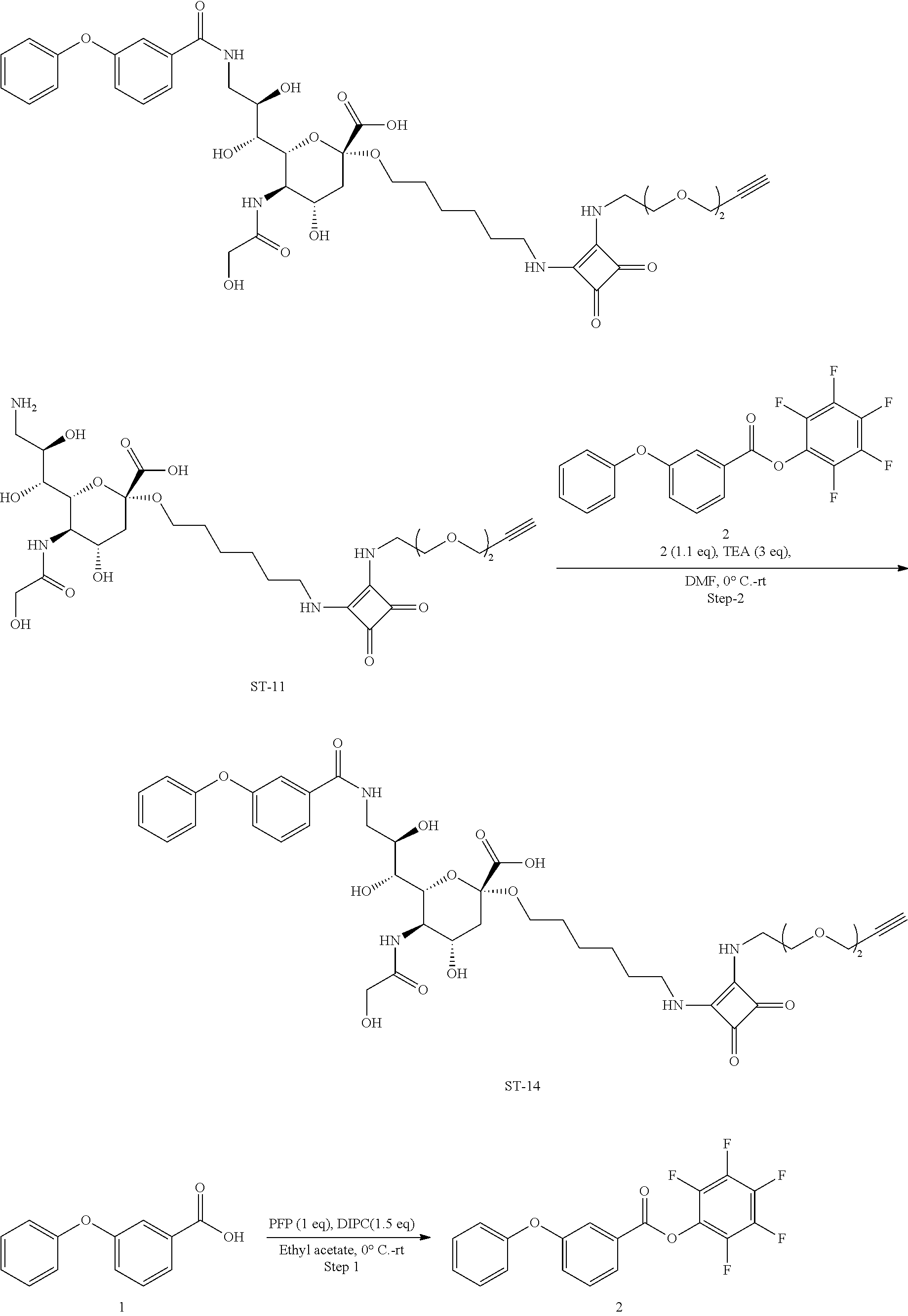

### Synthesis of perfluoro phenyl 3-phenoxybenzoate (2)

To a stirred solution of 3-phenoxybenzoic acid (1 g, 4.66 mmol) and penta-fluoro phenol (850 mg, 4.66 mmol) in dry ethyl acetate (10 mL) was added dropwise di-isopropyl carbodiimide (880 mg, 6.99 mmol) at 0° C. The reaction mixture was stirred at room temperature for 2 h. After completion, the reaction mixture was filtered and the precipitate was rinsed with ethyl acetate (20 mL). The filtrate was concentrated under reduced pressure and the crude residue obtained was purified by silica gel column chromatography using ethyl acetate and hexane as eluents to afford he desired product perfluoro phenyl 3-phenoxybenzoate (2) as white solid. Yield: 500 mg, 28%. ¹H NMR (400 MHz, d₆-DMSO) δ: 7.95 (d, J=8.0, 4H), 7.69 (t, J=8.0, 1H), 7.64 (s, 1H), 7.51-7.44 (m, 3H), 7.23 (t, J=8.0, 1H), 7.14 (d, J=8.0, 2H).

### Synthesis of (2R,4S,5R,6R)-6-((1R,2R)-1,2-dihydroxy-3-(3-phenoxy benzamido) propyl)-2-((6-((3,4-dioxo-2-((2-(2-(prop-2-yn-1-yloxy) ethoxy)ethyl)amino) cyclobut-1-en-1-yl)amino)hexyl) oxy)-4-hydroxy-5-(2-hydroxyacetamido) tetrahydro-2H-pyran-2-carboxylic acid (Cpd. 39)

To a stirred solution of (2R,4S,5R,6R)-6-((1R,2R)-3-amino-1,2-dihydroxypropyl)-2-((6-((3,4-dioxo-2-((2-(2-(prop-2-yn-1-yloxy)ethoxy)ethyl)amino)cyclobut-1-en-1-yl)amino) hexyl)oxy)-4-hydroxy-5-(2-hydroxyacetamido)tetrahydro-2H-pyran-2-carboxylic acid (ST-11) (100 mg, 0.15 mmol) in DMF (1 mL) was added perfluoro phenyl 3-phenoxybenzoate (64 mg, 0.17 mmol, 1.5 eq) at room temperature. After 10 minutes triethylamine (0.047 g, 0.1 mL, 59.7 mmol) was added dropwise at 0° C. The resulting reaction mixture was stirred at rt for 2 h. After completion the reaction mixture was purified by C18 reverse phase column chromatography to afford the desired product (2R,4S,5R,6R)-6-((1R,2R)-1,2-dihydroxy-3-(3-phenoxybenzamido) propyl)-2-((6-((3,4-dioxo-2-((2-(2-(prop-2-yn-1-yloxy) ethoxy)ethyl)amino) cyclobut-1-en-1-yl)amino)hexyl)oxy)-4-hydroxy-5-(2-hydroxyacetamido) tetrahydro-2H-pyran-2-carboxylic acid (39) as white solid. Yield: 67 mg, 51%. LCMS (ESI+) m/z 841.7 [M+H]⁺. ¹H NMR (400 MHz, MeOD) δ 7.59 (dt, J=8.0, 1.2 Hz, 1H), 7.54-7.35 (m, 4H), 7.16 (dd, J=7.4, 6.2, 2.4, 1.1 Hz, 2H), 7.07-7.00 (m, 2H), 4.19 (d, J=2.4 Hz, 2H), 4.04 (s, 3H), 4.04-3.98 (m, 1H), 3.87-3.73 (m, 4H), 3.72-3.61 (m, 5H), 3.61-3.35 (m, 2H), 2.87 (t, J=2.4 Hz, 1H), 2.76-2.66 (m, 1H), 1.75 (s, 1H), 1.59 (d, J=9.0 Hz, 5H), 1.39 (s, 5H).

### (2R,4S,5R,6R)-6-((1R,2R)-3-(2-([1,1′-biphenyl]-4-yl)acetamido)-1,2-dihydroxypropyl)-2-((6-((3,4-dioxo-2-(prop-2-yn-1-ylamino) cyclobut-1-en-1-yl)amino)hexyl)oxy)-4-hydroxy-5-ureidotetrahydro-2H-pyran-2-carboxylic acid (Compound 224)

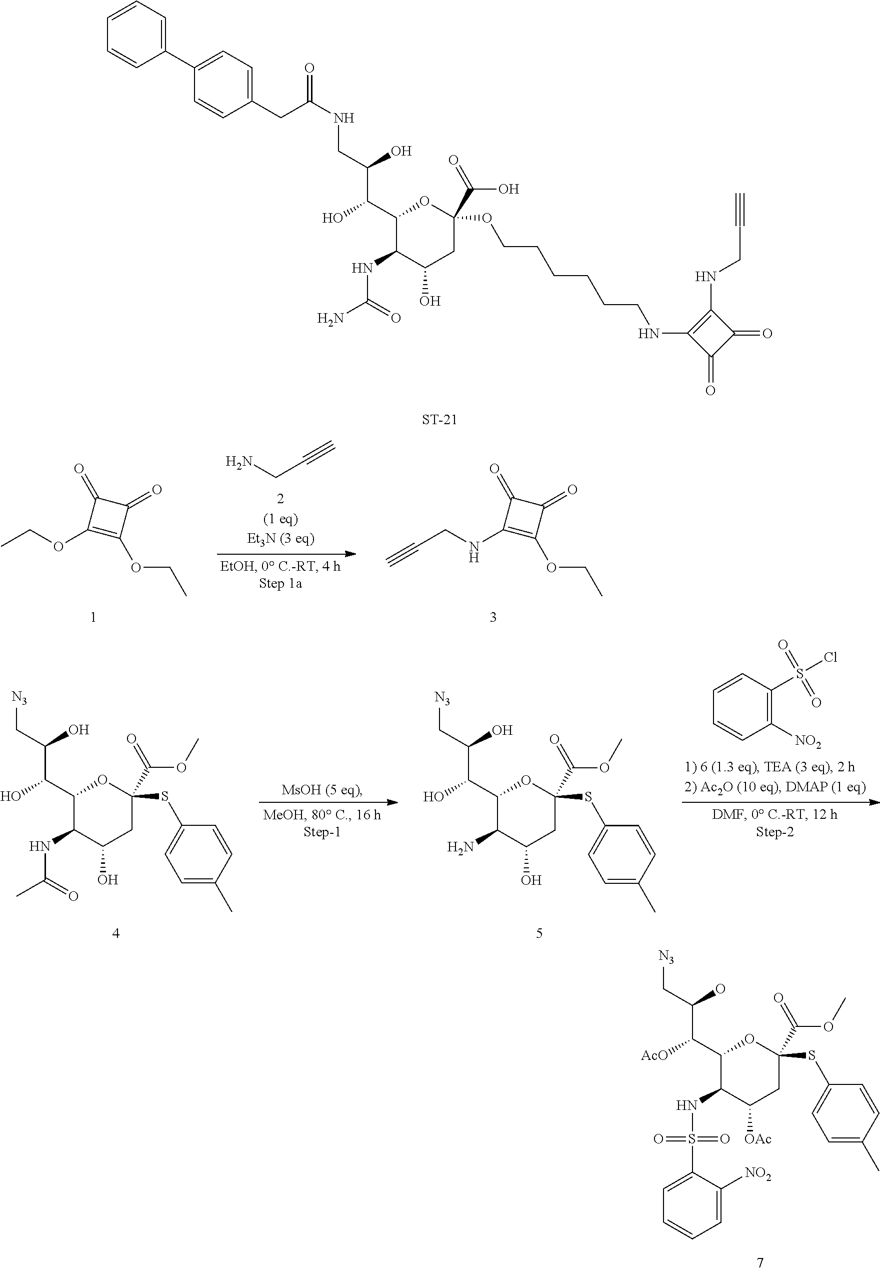

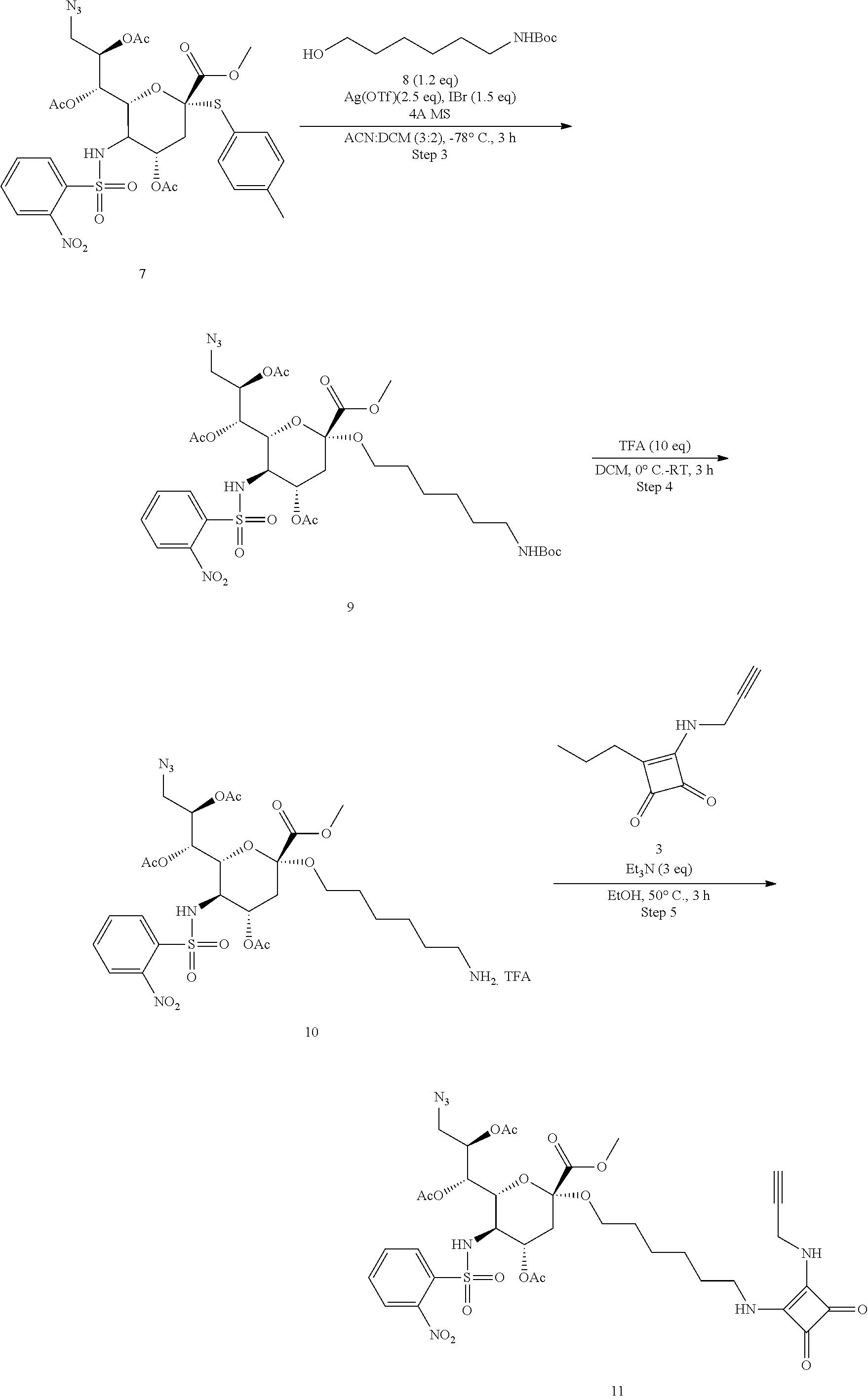

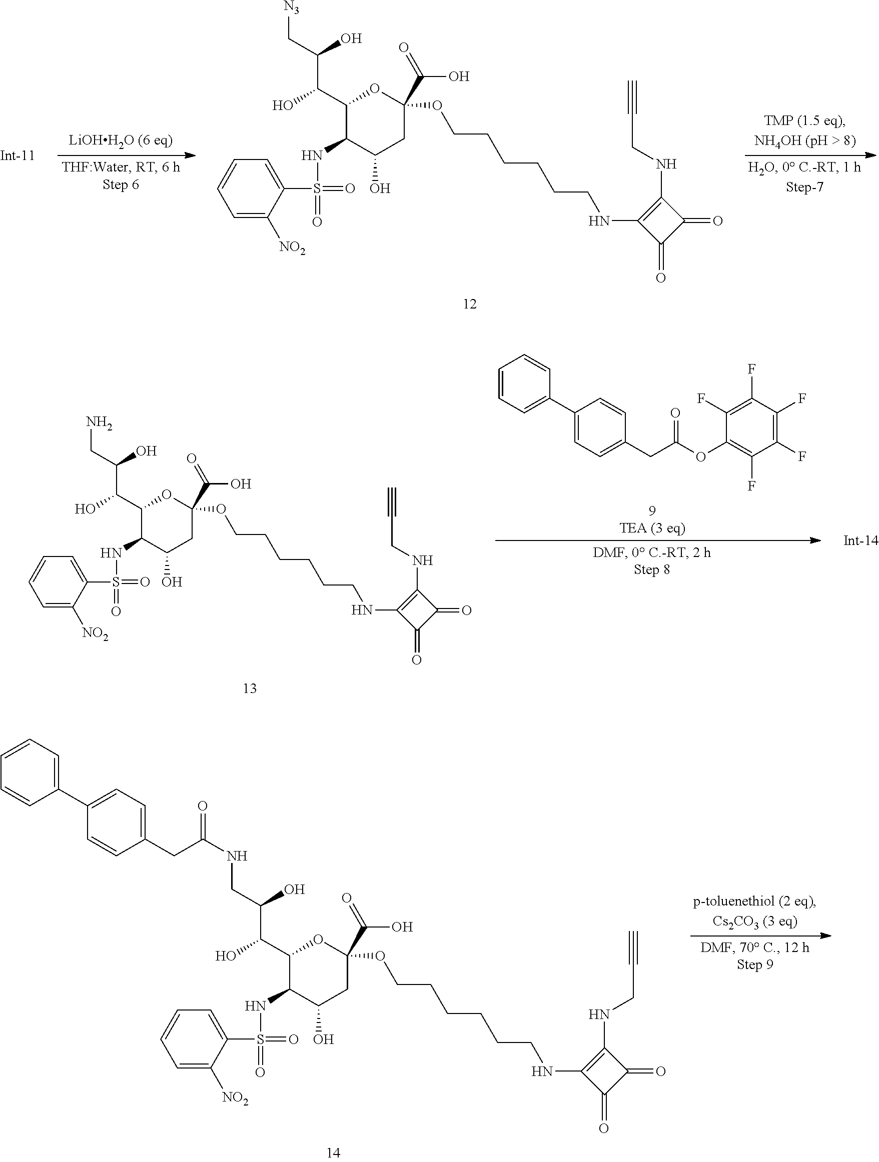 [SMILES: C#CCNc1c(NCCCCCCO[C@]2(C(=O)O)C[C@H](O)[C@@H](NS(=O)(=O)c3ccccc3[N+](=O)[O-])[C@H]([C@H](O)[C@H](O)CN=[N+]=[N-])O2)c(=O)c1=O ; C#CCNc1c(NCCCCCCO[C@]2(C(=O)O)C[C@H](O)[C@@H](NS(=O)(=O)c3ccccc3[N+](=O)[O-])[C@H]([C@H](O)[C@H](O)CN)O2)c(=O)c1=O ; O=C(Cc1ccc(-c2ccccc2)cc1)Oc1c(F)c(F)c(F)c(F)c1F ; C#CCNc1c(NCCCCCCO[C@]2(C(=O)O)C[C@H](O)[C@@H](NS(=O)(=O)c3ccccc3[N+](=O)[O-])[C@H]([C@H](O)[C@H](O)CNC(=O)Cc3ccc(-c4ccccc4)cc3)O2)c(=O)c1=O]

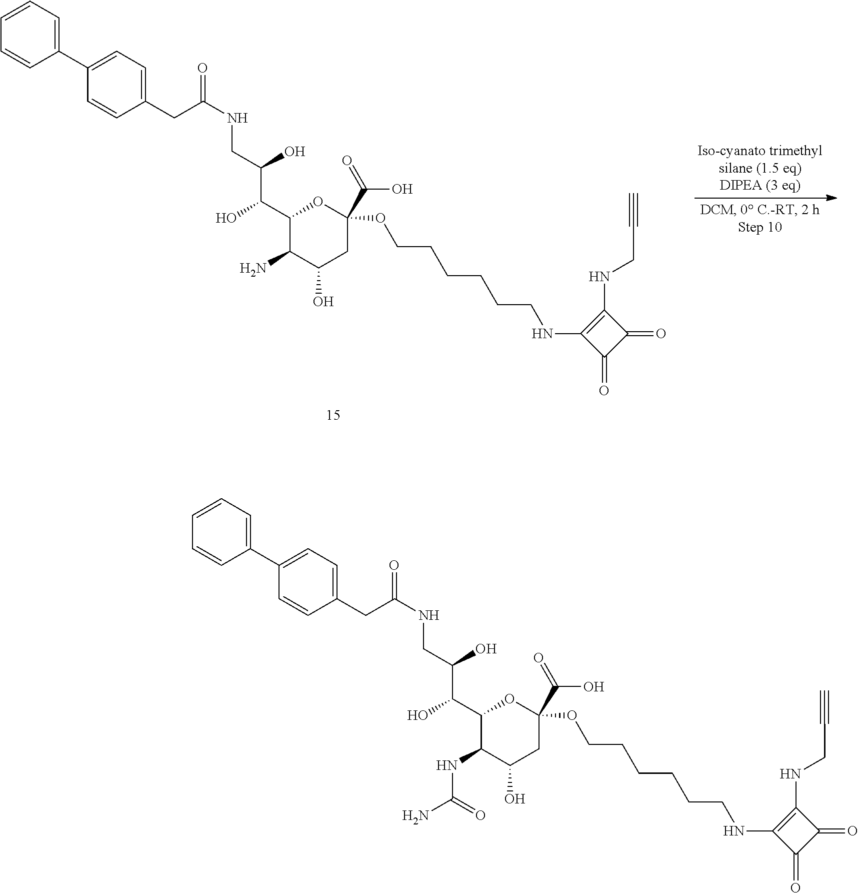 [SMILES: C#CCNc1c(NCCCCCCO[C@]2(C(=O)O)C[C@H](O)[C@@H](N)[C@H]([C@H](O)[C@H](O)CNC(=O)Cc3ccc(-c4ccccc4)cc3)O2)c(=O)c1=O ; C#CCNc1c(NCCCCCCO[C@]2(C(=O)O)C[C@H](O)[C@@H](NC(N)=O)[C@H]([C@H](O)[C@H](O)CNC(=O)Cc3ccc(-c4ccccc4)cc3)O2)c(=O)c1=O]

### Synthesis of 3-ethoxy-4-(prop-2-yn-1-ylamino) cyclobut-3-ene-1,2-dione (3)

To a stirred solution of 3,4-diethoxycyclobut-3-ene-1,2-dione (1) (3 g, 17.63 mmol) in ethanol (30 mL) was added prop-2-yn-1-amine (2) (971 mg, 17.63 mmol) at 0° C. followed by addition of triethylamine (7.37 mL, 52.89 mmol). The resulting reaction mixture was stirred at room temperature for 4 h. After completion, the reaction mixture was concentrated and the crude residue obtained was purified by silica-gel column chromatography using ethyl acetate and hexane as eluents to afford the desired product 3-ethoxy-4-(prop-2-yn-1-ylamino) cyclobut-3-ene-1,2-dione (3) as a yellow solid, Yield: 2.5 g, 79%; LCMS (ESI+) m/z 180.1 [M+H]⁺.

### Synthesis of methyl (2R,4S,5R,6R)-5-amino-6-((1R,2R)-3-azido-1,2-dihydroxypropyl)-4-hydroxy-2-(p-tolylthio) tetrahydro-2H-pyran-2-carboxylate (5)

To a stirred solution of methyl (2R,4S,5R,6R)-5-acetamido-6-((1R,2R)-3-azido-1,2-dihydroxypropyl)-4-hydroxy-2-(p-tolylthio) tetrahydro-2H-pyran-2-carboxylate (4) (5 g, 11 mmol) in methanol (150 mL) was added methane sulfonic acid (3.57 mL, 55.01 mmol) dropwise at 0° C., then the reaction mixture was heated at 80° C. for 16 h. After completion, the reaction mixture was quenched by addition of triethylamine at 0° C. maintaining neutral pH. The reaction mixture was concentrated to afford of the desired product methyl (2R,4S,5R,6R)-5-amino-6-((1R,2R)-3-azido-1,2-dihydroxypropyl)-4-hydroxy-2-(p-tolylthio) tetrahydro-2H-pyran-2-carboxylate (5), which was used as such for the next step, Yield: 4 g, 88%; LCMS (ESI+) m/z 413.4 [M+H]⁺.

### Synthesis of (1R,2R)-1-((2R,3R,4S,6R)-4-acetoxy-6-(methoxycarbonyl)-3-((2-nitrophenyl) sulfonamido)-6-(p-tolylthio) tetrahydro-2H-pyran-2-yl)-3-azidopropane-1,2-diyl diacetate (7)

To a stirred solution of methyl (2R,4S,5R,6R)-5-amino-6-((1R,2R)-3-azido-1,2-dihydroxy propyl)-4-hydroxy-2-(p-tolylthio) tetrahydro-2H-pyran-2-carboxylate (5) (4 g, 9.7 mmol) in N,N-dimethylformamide (40 ml) was added triethylamine (4 ml, 29.09 mmol) dropwise at 0° C. After 15 min of stirring, 2-nitrobenzenesulfonyl chloride (6) (2.79 g, 12.61 mmol) dissolved in N,N-dimethylformamide was added dropwise at 0° C. The reaction mixture was stirred at rt for 2 h. After completion, 4-dimethyl aminopyridine (1.18 g, 9.70 mmol) and acetic anhydride (9.17 ml, 96.98 mmol) were added dropwise at 0° C. over 10 min and the reaction mixture was stirred at room temperature for 12 h. After completion, the reaction mixture was diluted with cold water, extracted with ethyl acetate, and the organic layer was washed twice with ice cold water (250 mL), dried over anhydrous sodium sulfate, filtered and concentrated under reduced pressure. The crude residue obtained was purified by C18 reverse phase column chromatography using acetonitrile and water (+0.1% TFA) as eluents to afford the desired product (1R,2R)-1-((2R,3R,4S,6R)-4-acetoxy-6-(methoxycarbonyl)-3-((2-nitrophenyl)sulfonamido)-6-(p-tolylthio)tetrahydro-2H-pyran-2-yl)-3-azidopropane-1,2-diyl diacetate (7) as off white solid, Yield: 2.5 g, 35%; LCMS (ESI+) m/z 741.4 [M+H2O]⁺.

### Synthesis of (1R,2R)-1-((2R,3R,4S,6R)-4-acetoxy-6-((6-((tert-butoxy carbonyl)amino)hexyl) oxy)-6-(methoxycarbonyl)-3-((2-nitrophenyl) sulfonamido) tetrahydro-2H-pyran-2-yl)-3-azidopropane-1,2-diyl diacetate (9)

A solution (1R,2R)-1-((2R,3R,4S,6S)-4-acetoxy-6-(methoxycarbonyl)-3-((2-nitrophenyl)sulfonamido)-6-(p-tolylthio)tetrahydro-2H-pyran-2-yl)-3-azidopropane-1,2-diyl diacetate (7) (2 g, 2.76 mmol) and tert-butyl (6-hydroxyhexyl)carbamate (8) (720 mg, 3.32 mmol), Silver (1+) trifluoro methane sulfonate (AgOTf) (1.78 g, 6.91 mmol) and activated 4 Å powdered molecular sieves (5 g) in anhydrous dichloromethane (12 mL) and anhydrous acetonitrile (24 mL) was stirred at room temperature for 1 h under nitrogen. The solution was cooled to −78° C., iodine monobromide (857 mg, 4.15 mmol) solution in dichloromethane (4 mL) was added dropwise, and the resulting mixture was stirred at same temperature for 2 h. After completion, the reaction mixture was quenched by addition of triethylamine (5 mL) and warmed to room temperature. The reaction mixture was filtered, the filtrate was washed with a saturated aqueous solution of sodium thiosulfate, dried over sodium sulfate, filtered and concentrated under reduced pressure. The crude residue obtained was purified by C18 reverse phase column chromatography using water: acetonitrile as eluents to afford the desired product (1R,2R)-1-((2R,3R,4S,6R)-4-acetoxy-6-((6-((tert-butoxy carbonyl)amino)hexyl)oxy)-6-(methoxy carbonyl)-3-((2-nitrophenyl) sulfonamido) tetrahydro-2H-pyran-2-yl)-3-azidopropane-1,2-diyl diacetate (9) as a pale yellow solid, Yield: 1 g, 44%; LCMS (ESI−) m/z 815.7 [M−H]⁻.

### Synthesis of (1R,2R)-1-((2R,3R,4S,6R)-4-acetoxy-6-((6-aminohexyl)oxy)-6-(methoxycarbonyl)-3-((2-nitrophenyl) sulfonamido) tetrahydro-2H-pyran-2-yl)-3-azidopropane-1,2-diyl diacetate TFA salt (10)

To a stirred solution of (1R,2R)-1-((2R,3R,4S,6R)-4-acetoxy-6-((6-((tert-butoxy carbonyl)amino)hexyl)oxy)-6-(methoxycarbonyl)-3-((2-nitrophenyl)sulfonamido)tetrahydro-2H-pyran-2-yl)-3-azidopropane-1,2-diyl diacetate (9) (1 g, 1.22 mmol) in anhydrous dichloromethane (10 mL) was added trifluoro acetic acid (0.9 mL) dropwise at 0° C. and the reaction mixture was stirred at room temperature for 3 h. After completion, the solvent was concentrated to afford the desired product (1R,2R)-1-((2R,3R,4S,6R)-4-acetoxy-6-((6-aminohexyl)oxy)-6-(methoxycarbonyl)-3-((2-nitrophenyl) sulfonamido) tetrahydro-2H-pyran-2-yl)-3-azidopropane-1,2-diyl diacetate TFA salt (10) as a light-yellow sticky syrup. The crude residue was directly used for the next step without further purification, Yield: 800 mg, Crude; LCMS (ESI+) m/z 717.5 [M+H]⁺.

### Synthesis of (1R,2R)-1-((2R,3R,4S,6R)-4-acetoxy-6-((6-((3,4-dioxo-2-(prop-2-yn-1-ylamino)cyclobut-1-en-1-yl)amino)hexyl)oxy)-6-(methoxycarbonyl)-3-((2-nitrophenyl) sulfonamido) tetrahydro-2H-pyran-2-yl)-3-azidopropane-1,2-diyl diacetate (11)

To a stirred solution of (1R,2R)-1-((2R,3R,4S,6R)-4-acetoxy-6-((6-aminohexyl)oxy)-6-(methoxycarbonyl)-3-((2-nitrophenyl)sulfonamido)tetrahydro-2H-pyran-2-yl)-3-azidopropane-1,2-diyl diacetate TFA salt (10) (0.8 g, 0.98 mmol) in ethanol (5 mL), was added triethylamine (0.55 mL, 3.93 mmol). After 10 minutes 3-ethoxy-4-(prop-2-yn-1-ylamino) cyclobut-3-ene-1,2-dione (3), 0.18 g, 0.98 mmol) was added at room temperature, then the resulting reaction mixture was stirred at 50° C. for 3 h. After completion, the reaction mixture was concentrated and the crude residue obtained was purified by C18 reverse phase column chromatography using acetonitrile and water (+0.05% TFA) as eluents to afford the desired product (1R,2R)-1-((2R,3R,4S,6R)-4-acetoxy-6-((6-((3,4-dioxo-2-(prop-2-yn-1-ylamino) cyclobut-1-en-1-yl)amino)hexyl)oxy)-6-(methoxycarbonyl)-3-((2-nitrophenyl) sulfonamido)tetrahydro-2H-pyran-2-yl)-3-azidopropane-1,2-diyl diacetate (11) as yellow solid, Yield: 500 mg, 60%; LCMS (ESI+) m/z 850.8 [M+H]⁺.

### Synthesis of (2R,4S,5R,6R)-6-((1R,2R)-3-azido-1,2-dihydroxypropyl)-2-((6-((3,4-dioxo-2-(prop-2-yn-1-ylamino) cyclobut-1-en-1-yl)amino)hexyl)oxy)-4-hydroxy-5-((2-nitrophenyl) sulfonamido) tetrahydro-2H-pyran-2-carboxylic acid (12)

To a stirred solution (1R,2R)-1-((2R,3R,4S,6R)-4-acetoxy-6-((6-((3,4-dioxo-2-(prop-2-yn-1-ylamino)cyclobut-1-en-1-yl)amino)hexyl)oxy)-6-(methoxycarbonyl)-3-((2-nitrophenyl) sulfon amido)tetrahydro-2H-pyran-2-yl)-3-azidopropane-1,2-diyl diacetate (11) (0.5 g, 0.59 mmol) in tetrahydrofuran (5 mL) was added a solution of lithium hydroxide monohydrate (148 mg, 3.53 mmol) in water (2 mL). The reaction mixture was stirred at room temperature for 6 h. After completion, the reaction mixture was treated with acidic resin (Dowex 50, H⁺) to pH˜6 and the suspension was filtered. The filtrate was concentrated and the residue material (2R,4S,5R,6R)-6-((1R,2R)-3-azido-1,2-dihydroxypropyl)-2-((6-((3,4-dioxo-2-(prop-2-yn-1-ylamino)cyclobut-1-en-1-yl)amino)hexyl)oxy)-4-hydroxy-5-((2-nitrophenyl)sulfon-amido)tetrahydro-2H-pyran-2-carboxylic acid (12) used as such without further purification, Yield: 400 mg, Crude; LCMS (ESI) m/z 710.4 [M+H]⁺.

### Synthesis of (2R,4S,5R,6R)-6-((1R,2R)-3-amino-1,2-dihydroxypropyl)-2-((6-((3,4-dioxo-2-(prop-2-yn-1-ylamino) cyclobut-1-en-1-yl)amino)hexyl)oxy)-4-hydroxy-5-((2-nitrophenyl) sulfonamido) tetrahydro-2H-pyran-2-carboxylic acid (13)

To a stirred solution of (2R,4S,5R,6R)-6-((1R,2R)-3-azido-1,2-dihydroxypropyl)-2-((6-((3,4-dioxo-2-(prop-2-yn-1-ylamino)cyclobut-1-en-1-yl)amino)hexyl)oxy)-4-hydroxy-5-((2-nitro phenyl)sulfonamido)tetrahydro-2H-pyran-2-carboxylic acid (12) (0.4 g, 0.56 mmol) in water (6 mL), NH₄OH (0.1 mL, pH>8) was added TMP (0.845 mL, 1.5 eq, 1M soln in THF) at 0° C. and the reaction mixture was stirred for 1 h at the same temperature. After completion, the reaction mixture was washed with ethyl acetate, and the aqueous phase was dried by lyophilization to get the desired product (2R,4S,5R,6R)-6-((1R,2R)-3-amino-1,2-dihydroxypropyl)-2-((6-((3,4-dioxo-2-(prop-2-yn-1-ylamino)cyclobut-1-en-1-yl)amino)hexyl)oxy)-4-hydroxy-5-((2-nitro phenyl)sulfonamido)tetrahydro-2H-pyran-2-carboxylic acid (13) as a white solid, which was directly used in the next step without further purification, Yield: 350 mg, Crude; LCMS (ESI+) m/z 684.4 [M+H]⁺.

### Synthesis of (2R,4S,5R,6R)-6-((1R,2R)-3-(2-([1,1′-biphenyl]-4-yl)acetamido)-1,2-dihydroxy propyl)-2-((6-((3,4-dioxo-2-(prop-2-yn-1-ylamino)cyclobut-1-en-1-yl)amino)hexyl)oxy)-4-hydroxy-5-((2-nitrophenyl)sulfonamido)tetrahydro-2H-pyran-2-carboxylic acid (14)

To a stirred solution of (2R,4S,5R,6R)-6-((1R,2R)-3-amino-1,2-dihydroxypropyl)-2-((6-((3,4-dioxo-2-(prop-2-yn-1-ylamino)cyclobut-1-en-1-yl)amino)hexyl)oxy)-4-hydroxy-5-((2-nitro phenyl)sulfonamido)tetrahydro-2H-pyran-2-carboxylic acid (13) (350 mg, 0.511 mmol) in DMF (2 mL) was added penta-fluorophenyl 2-([1,1′-biphenyl]-4-yl)acetate (9) (213 mg, 0.56 mmol, 1.1 eq) at room temperature. After 10 minutes, triethylamine (0.21 mL, 1.54 mmol) was added dropwise at 0° C. The resulting reaction mixture was stirred at room temperature for 2 h. After completion the reaction mixture was purified by C18 reverse phase column chromatography to afford the desired product (2R,4S,5R,6R)-6-((1R,2R)-3-(2-([1,1′-biphenyl]-4-yl)acetamido)-1,2-dihydroxypropyl)-2-((6-((3,4-dioxo-2-(prop-2-yn-1-ylamino)cyclobut-1-en-1-yl)amino)hexyl)oxy)-4-hydroxy-5-((2-nitrophenyl)sulfonamido) tetrahydro-2H-pyran-2-carboxylic acid (14) as white solid, Yield:70 mg, 16%; LCMS (ESI+) m/z 878.8 [M+H]⁺

### Synthesis of (2R,4S,5R,6R)-6-((1R,2R)-3-(2-([1,1′-biphenyl]-4-yl) acetamido)-1,2-dihydroxy propyl)-5-amino-2-((6-((3,4-dioxo-2-(prop-2-yn-1-ylamino) cyclobut-1-en-1-yl)amino)hexyl) oxy)-4-hydroxy tetrahydro-2H-pyran-2-carboxylic acid (15)

To a stirred solution of (2R,4S,5R,6R)-6-((1R,2R)-3-(2-([1,1′-biphenyl]-4-yl)acetamido)-1,2-dihydroxypropyl)-2-((6-((3,4-dioxo-2-(prop-2-yn-1-ylamino)cyclobut-1-en-1-yl)amino)hexyl)oxy)-4-hydroxy-5-((2-nitrophenyl)sulfonamido)tetrahydro-2H-pyran-2-carboxylic acid (14), (70 mg, 0.079 mmol) in anhydrous N,N-dimethylformamide (1 mL), 4-methylbenzenethiol (20 mg, 0.159 mmol) and cesium carbonate (83 mg, 0.255 mmol) were added at 0° C. The reaction mixture was stirred at 70° C. for 12 h. After completion, the reaction mixture was purified by C18 reverse phase chromatography using water and acetonitrile (+0.05% TFA) as eluents to afford the desired product (2R,4S,5R,6R)-6-((1R,2R)-3-(2-([1,1′-biphenyl]-4-yl)acetamido)-1,2-dihydroxy propyl)-5-amino-2-((6-((3,4-dioxo-2-(prop-2-yn-1-ylamino)cyclobut-1-en-1-yl)amino)hexyl)oxy)-4-hydroxy tetrahydro-2H-pyran-2-carboxylic acid (15) as off white solid, Yield: 15 mg, 27%; LCMS (ESI+) m/z 693.1 [M+H]⁺.

### Synthesis of (2R,4S,5R,6R)-6-((1R,2R)-3-(2-([1,1′-biphenyl]-4-yl) acetamido)-1,2-dihydroxy propyl)-2-((6-((3,4-dioxo-2-(prop-2-yn-1-ylamino) cyclobut-1-en-1-yl)amino)hexyl)oxy)-4-hydroxy-5-ureidotetrahydro-2H-pyran-2-carboxylic acid (Cpd. 224)

To a stirred solution of (2R,4S,5R,6R)-6-((1R,2R)-3-(2-([1,1′-biphenyl]-4-yl)acetamido)-1,2-dihydroxypropyl)-5-amino-2-((6-((3,4-dioxo-2-(prop-2-yn-1-ylamino)cyclobut-1-en-1-yl)amino)hexyl)oxy)-4-hydroxytetrahydro-2H-pyran-2-carboxylic acid (15) (15 mg, 0.02 mmol) in anhydrous dichloromethane (1 mL) was added isocyanato trimethyl silane (3.74 mg, 0.06 mmol) in dichloromethane (0.1 ml) at 0° C. followed by addition of N,N-diisopropylethylamine (0.01 mL, 0.06 mmol) and the reaction mixture was stirred at room temperature for 2 h. After completion the reaction mixture was concentrated and the crude residue obtained was purified by C18 preparative hplc using water and acetonitrile (+0.05% TFA) as eluents to afford the desired product (2R,4S,5R,6R)-6-((1R,2R)-3-(2-([1,1′-biphenyl]-4-yl)acetamido)-1,2-dihydroxypropyl)-2-((6-((3,4-dioxo-2-(prop-2-yn-1-ylamino) cyclobut-1-en-1-yl)amino)hexyl)oxy)-4-hydroxy-5-ureidotetrahydro-2H-pyran-2-carboxylic acid (Cpd. 224) as a white solid, Yield: 4.45 mg, 28%; LCMS (ESI+) m/z 736.8 [M+H]⁺.

¹H NMR (401 MHz, MeOD) δ 7.59 (ddt, J=10.4, 8.3, 1.8 Hz, 4H), 7.47-7.37 (m, 4H), 7.35-7.28 (m, 1H), 4.43 (s, 2H), 3.93 (s, 1H), 3.80-3.71 (m, 2H), 3.66-3.56 (m, 4H), 3.50 (d, J=16.5 Hz, 3H), 3.21 (dd, J=13.5, 8.4 Hz, 1H), 2.85-2.73 (m, 2H), 1.65 (t, J=11.9 Hz, 2H), 1.55 (s, 4H), 1.33 (d, J=15.4 Hz, 4H), 0.9 (s, 1H).

### (2R,4S,5R,6R)-6-((1R,2R)-1,2-dihydroxy-3-(3-phenoxybenzamido) propyl)-4-hydroxy-5-(2-hydroxyacetamido)-2-(oct-7-yn-1-yloxy) tetrahydro-2H-pyran-2-carboxylic acid (Compound 40)

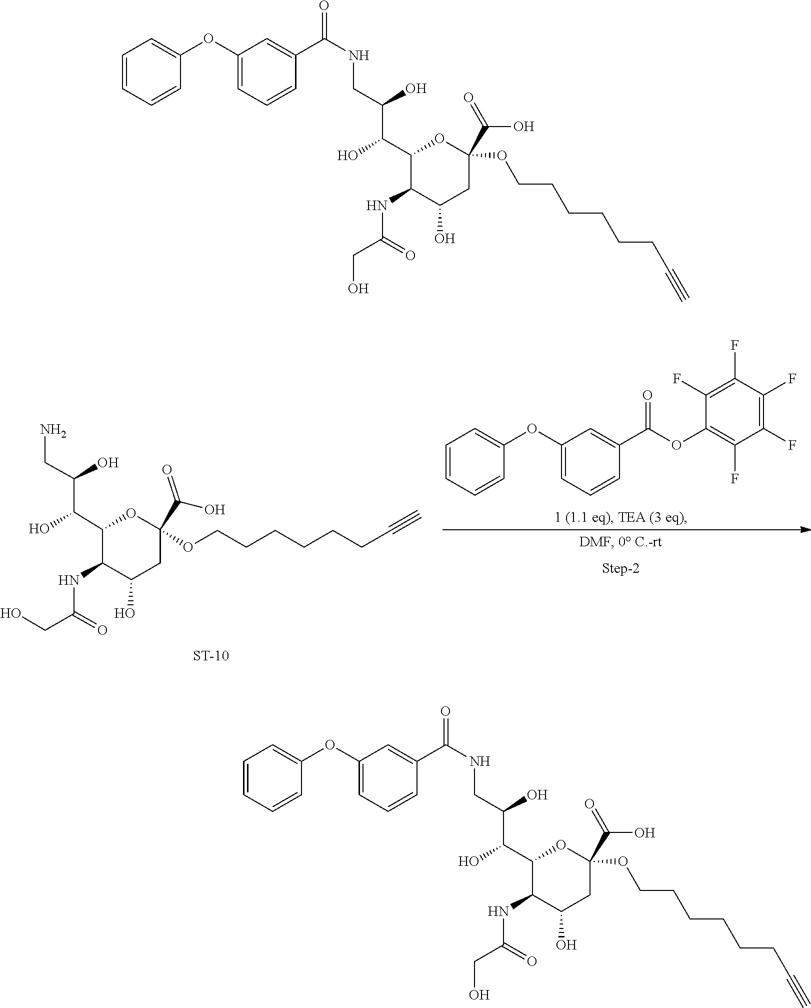

### Synthesis of (2R,4S,5R,6R)-6-((1R,2R)-1,2-dihydroxy-3-(3-phenoxybenzamido) propyl)-4-hydroxy-5-(2-hydroxyacetamido)-2-(oct-7-yn-1-yloxy) tetrahydro-2H-pyran-2-carboxylic acid (Cpd. 40)

To a stirred solution of (2R,4S,5R,6R)-6-((1R,2R)-3-amino-1,2-dihydroxypropyl)-4-hydroxy-5-(2-hydroxyacetamido)-2-(oct-7-yn-1-yloxy)tetrahydro-2H-pyran-2-carboxylic acid (ST-10) (100 mg, 0.23 mmol) in DMF (1 mL) was added perfluoro phenyl 3-phenoxybenzoate (96 mg, 0.25 mmol, 1.5 eq) at room temperature. After 10 minutes triethyl amine (7 mg, 0.1 mL, 0.69 mmol) was added dropwise at 0° C. The resulting reaction mixture was stirred at rt for 2 h. After completion the reaction mixture was purified by C18 reverse phase column chromatography to afford the desired product (2R,4S,5R,6R)-6-((1R,2R)-1,2-dihydroxy-3-(3-phenoxybenzamido) propyl)-4-hydroxy-5-(2-hydroxyacetamido)-2-(oct-7-yn-1-yloxy) tetrahydro-2H-pyran-2-carboxylic acid (40) as white solid. Yield: 88 mg, 60%. LCMS (ESI+) m/z 629.6 [M+H]⁺. ¹H NMR (400 MHz, MeOD) δ 7.59 (dt, J=7.8, 1.2 Hz, 1H), 7.52-7.35 (m, 4H), 7.16 (tt, J=7.3, 1.2 Hz, 2H), 7.08-7.00 (m, 2H), 4.03 (s, 3H), 3.92-3.73 (m, 5H), 3.58-3.35 (m, 3H), 2.73 (dd, J=12.8, 3.5 Hz, 1H), 2.21-2.11 (m, 3H), 1.81-1.70 (m, 1H), 1.57-1.34 (m, 8H).

### (2R,4S,5R,6R)-6-((1R,2R)-3-(2,2-difluoro-2-phenylacetamido)-1,2-dihydroxypropyl)-2-((6-((3,4-dioxo-2-((2-(2-(prop-2-yn-1-yloxy) ethoxy)ethyl)amino) cyclobut-1-en-1-yl)amino) hexyl)oxy)-4-hydroxy-5-(2-hydroxyacetamido) tetrahydro-2H-pyran-2-carboxylic acid (Cpd. 275)

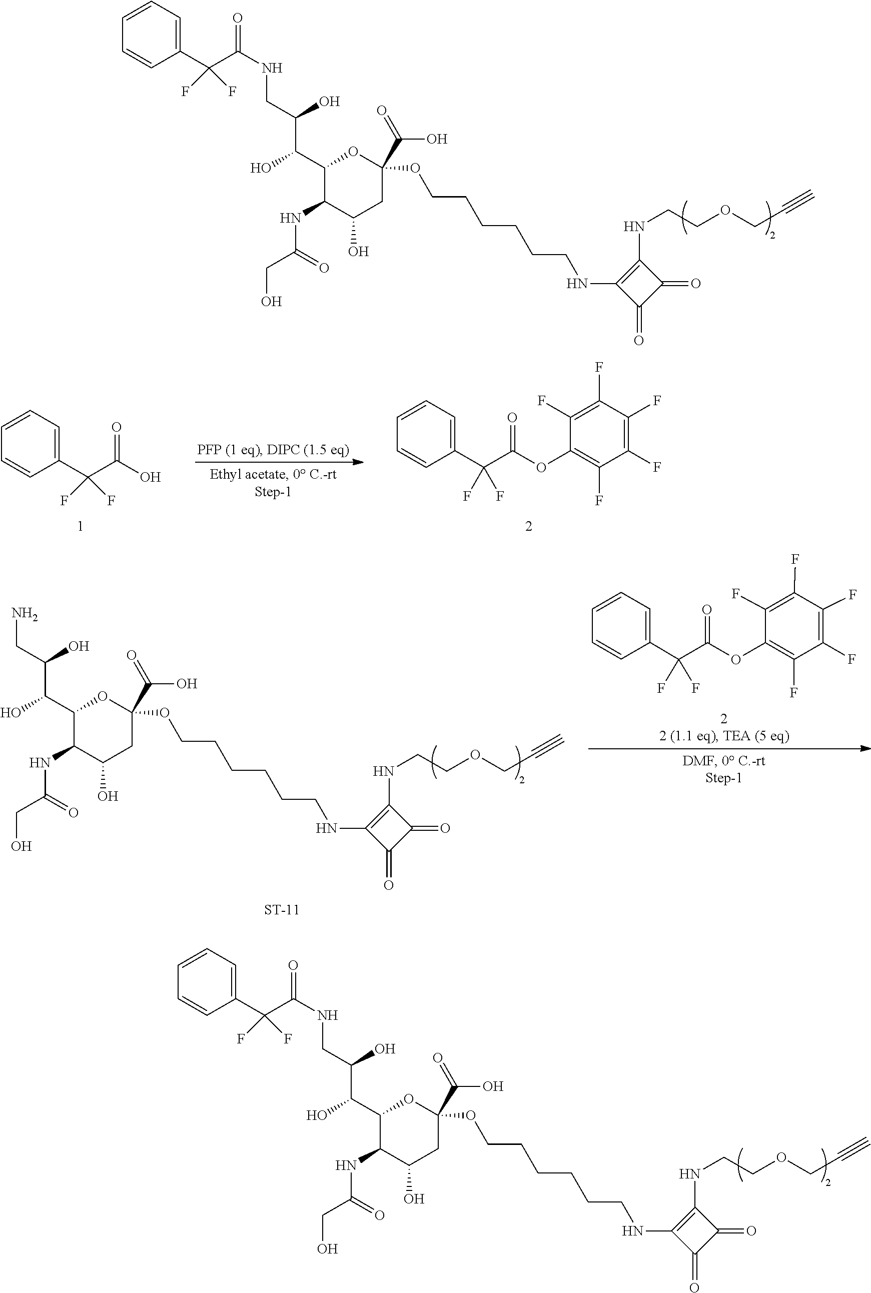

### Synthesis of perfluoro phenyl 2,2-difluoro-2-phenylacetate (2)

To a stirred solution of 2,2-difluoro-2-phenylacetic acid (2 g, 11.6 mmol) and penta-fluoro phenol (2.13 g, 11.6 mmol) in dry ethyl acetate (20 mL) was added dropwise diisopropyl carbodiimide (2.08 g, 16.5 mmol) at 0° C. The reaction mixture was stirred at room temperature for 2 h. After completion, the reaction mixture was filtered and the filter cake was rinsed with ethyl acetate (40 mL). The filtrate was concentrated under reduced pressure and the crude residue obtained was purified by silica gel column chromatography, with ethyla acetate and hexanes as eluents to afford the desired product perfluoro phenyl 2,2-difluoro-2-phenylacetate (2) as off white semi solid. Yield-500 mg, 12%. ¹H NMR (400 MHz, DMSO) δ:7.74-7.50 (m, 5H).

### Synthesis of (2R,4S,5R,6R)-6-((1R,2R)-3-(2,2-difluoro-2-phenylacetamido)-1,2-dihydroxypropyl)-2-((6-((3,4-dioxo-2-((2-(2-(prop-2-yn-1-yloxy) ethoxy)ethyl)amino) cyclobut-1-en-1-yl)amino)hexyl)oxy)-4-hydroxy-5-(2-hydroxyacetamido) tetrahydro-2H-pyran-2-carboxylic acid (Cpd. 41)

To a stirred solution of (2R,4S,5R,6R)-6-((1R,2R)-3-amino-1,2-dihydroxypropyl)-2-((6-((3,4-dioxo-2-((2-(2-(prop-2-yn-1-yloxy)ethoxy)ethyl)amino)cyclobut-1-en-1-yl)amino) hexyl)oxy)-4-hydroxy-5-(2-hydroxyacetamido)tetrahydro-2H-pyran-2-carboxylic acid (ST-11) (100 mg, 0.155 mmol) in DMF (1 mL) was added perfluoro phenyl 2,2-difluoro-2-phenylacetate (78 mg, 0.23 mmol, 1.5 eq) at room temperature. After 10 minutes triethylamine (78 mg, 0.77 mmol, 5 eq) was added at 0° C. The resulting reaction mixture was stirred at rt for 2 h. After completion, the reaction mixture was purified by prep-HPLC to afford the desired product (2R,4S,5R,6R)-6-((1R,2R)-3-(2,2-difluoro-2-phenylacetamido)-1,2-dihydroxypropyl)-2-((6-((3,4-dioxo-2-((2-(2-(prop-2-yn-1-yloxy) ethoxy)ethyl)amino) cyclobut-1-en-1-yl)amino) hexyl)oxy)-4-hydroxy-5-(2-hydroxyacetamido) tetrahydro-2H-pyran-2-carboxylic acid (Cpd. 275) as white solid. Yield: 56 mg, 45%. LCMS (ESI+) m/z 799.6 [M+H]⁺. ¹H NMR (400 MHz, MeOD): δ 8.72 (s, 1H), 7.93 (d, J=7.8 Hz, 1H), 7.69-7.62 (m, 2H), 7.57-7.45 (m, 3H), 4.23-4.16 (m, 2H), 4.04 (s, 2H), 3.96 (td, J=8.4, 3.2 Hz, 1H), 3.91-3.72 (m, 1H), 3.75-3.62 (m, 13H), 3.47-3.37 (m, 3H), 2.87 (t, J=2.4 Hz, 1H), 2.73 (dd, J=12.7, 4.1 Hz, 1H), 1.74 (t, J=11.9 Hz, 1H), 1.64-1.54 (m, 4H), 1.51-1.39 (m, 4H).

### (2R,4S,5R,6R)-6-((1R,2R)-3-(2,2-difluoro-2-phenylacetamido)-1,2-dihydroxypropyl)-4-hydroxy-5-(2-hydroxyacetamido)-2-(oct-7-yn-1-yloxy) tetrahydro-2H-pyran-2-carboxylic acid (Compound 42)

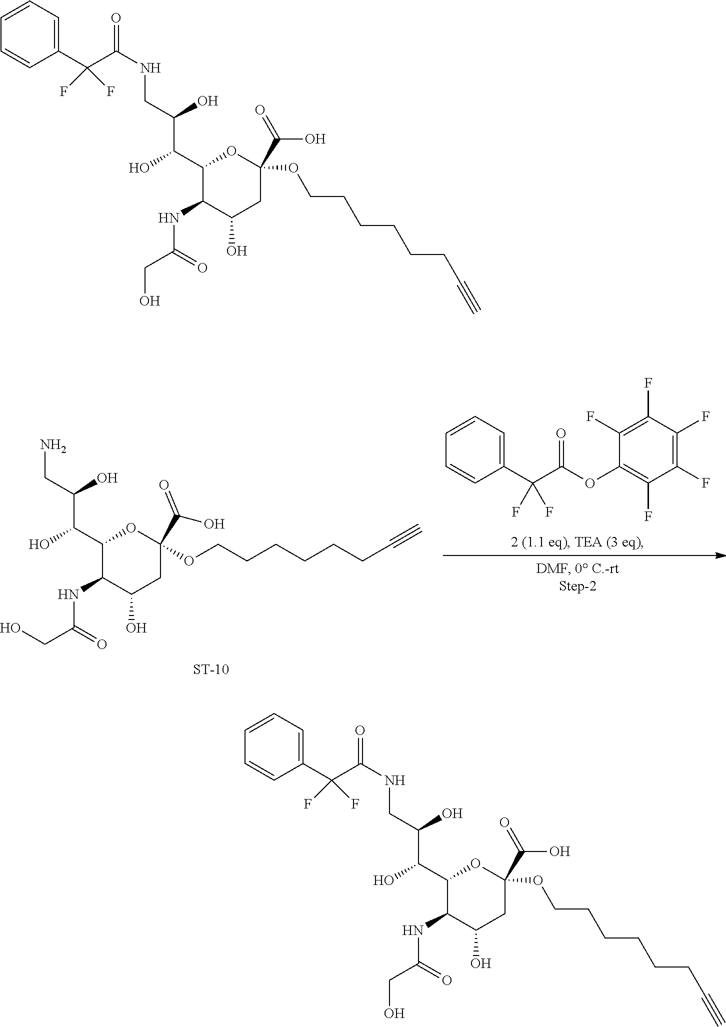 [SMILES: C#CCCCCCCO[C@]1(C(=O)O)C[C@H](O)[C@@H](NC(=O)CO)[C@H]([C@H](O)[C@H](O)CNC(=O)C(F)(F)c2ccccc2)O1 ; C#CCCCCCCO[C@]1(C(=O)O)C[C@H](O)[C@@H](NC(=O)CO)[C@H]([C@H](O)[C@H](O)CN)O1 ; O=C(Oc1c(F)c(F)c(F)c(F)c1F)C(F)(F)c1ccccc1 ; "ST-10" ; C#CCCCCCCO[C@]1(C(=O)O)C[C@H](O)[C@@H](NC(=O)CO)[C@H]([C@H](O)[C@H](O)CNC(=O)C(F)(F)c2ccccc2)O1]

### Synthesis of (2R,4S,5R,6R)-6-((1R,2R)-3-(2,2-difluoro-2-phenylacetamido)-1,2-dihydroxypropyl)-4-hydroxy-5-(2-hydroxyacetamido)-2-(oct-7-yn-1-yloxy) tetrahydro-2H-pyran-2-carboxylic acid (Cpd. 42)

To a stirred solution of (2R,4S,5R,6R)-6-((1R,2R)-3-amino-1,2-dihydroxypropyl)-4-hydroxy-5-(2-hydroxyacetamido)-2-(oct-7-yn-1-yloxy)tetrahydro-2H-pyran-2-carboxylic acid (ST-10) (100 mg, 0.23 mmol) in DMF (1 mL) was added perfluoro phenyl 2,2-difluoro-2-phenylacetate (117 mg, 0.34 mmol, 1.5 eq) at room temperature. After 10 minutes triethylamine (116 mg, 1.15 mmol) was added dropwise at 0° C. The resulting reaction mixture was stirred at rt for 2 h. After completion the reaction mixture was purified by C18 reverse phase column chromatography to afford the desired product (2R,4S,5R,6R)-6-((1R,2R)-3-(2,2-difluoro-2-phenylacetamido)-1,2-dihydroxypropyl)-4-hydroxy-5-(2-hydroxyacetamido)-2-(oct-7-yn-1-yloxy) tetrahydro-2H-pyran-2-carboxylic acid (42), as white solid. Yield: 55 mg, 41%. LCMS (ESI+) m/z 587.6 [M+H]⁺. ¹H NMR (400 MHz, Methanol-d₄): δ 8.67 (s, 1H), 7.93 (d, J=7.6 Hz, 1H), 7.69-7.62 (m, 2H), 7.56-7.45 (m, 3H), 4.03 (s, 2H), 3.96 (td, J=8.2, 3.3 Hz, 1H), 3.88-3.65 (m, 5H), 3.47-3.38 (m, 3H), 2.74 (dd, J=12.7, 4.1 Hz, 1H), 2.22-2.13 (m, 3H), 1.74 (t, J=11.9 Hz, 1H), 1.57-1.33 (m, 8H).

### (2R,4S,5R,6R)-6-((1R,2R)-3-(2-([1,1′-biphenyl]-4-yl) acetamido)-1,2-dihydroxypropyl)-2-((6-((3,4-dioxo-2-(prop-2-yn-1-ylamino) cyclobut-1-en-1-yl)amino)hexyl)oxy)-4-hydroxy-5-ureidotetrahydro-2H-pyran-2-carboxylic acid (Compound 224)

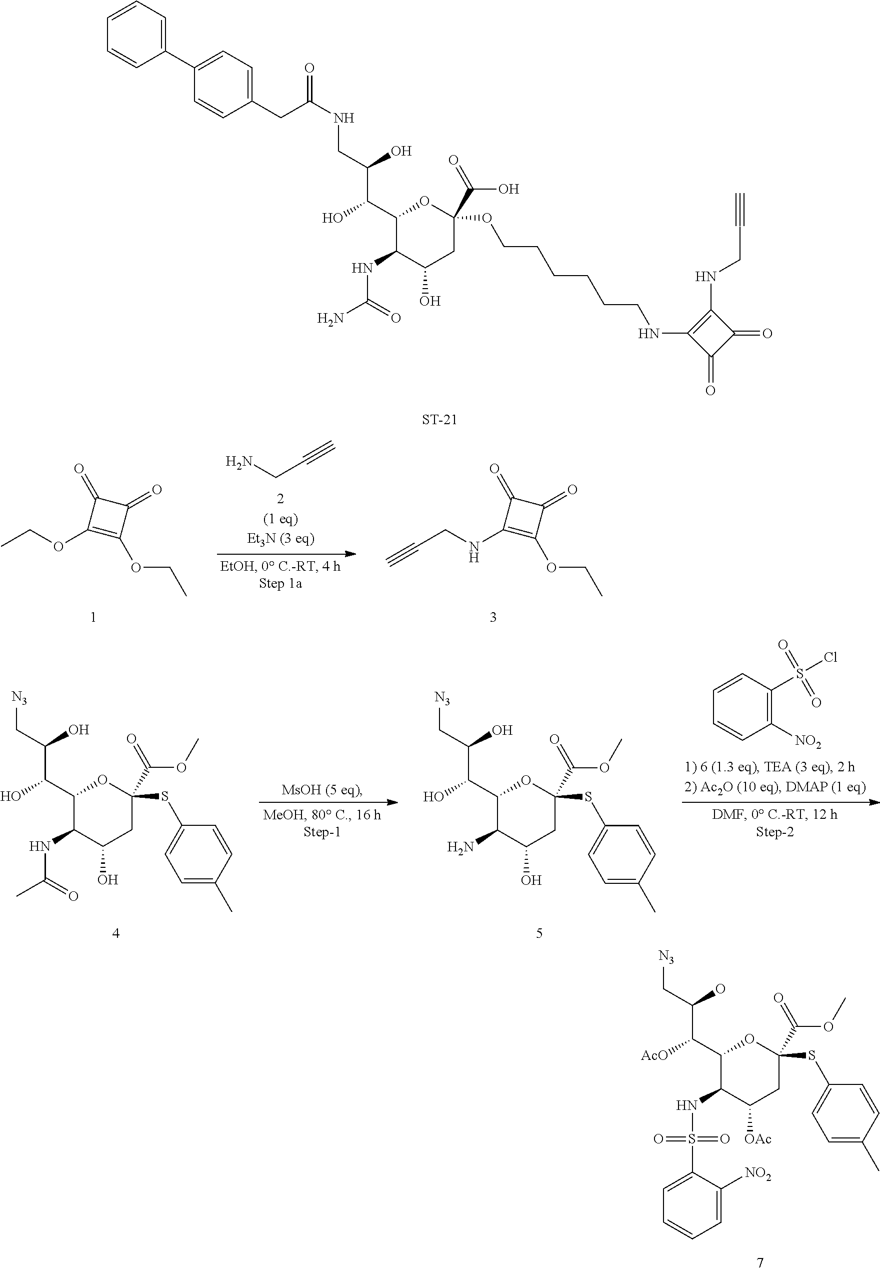

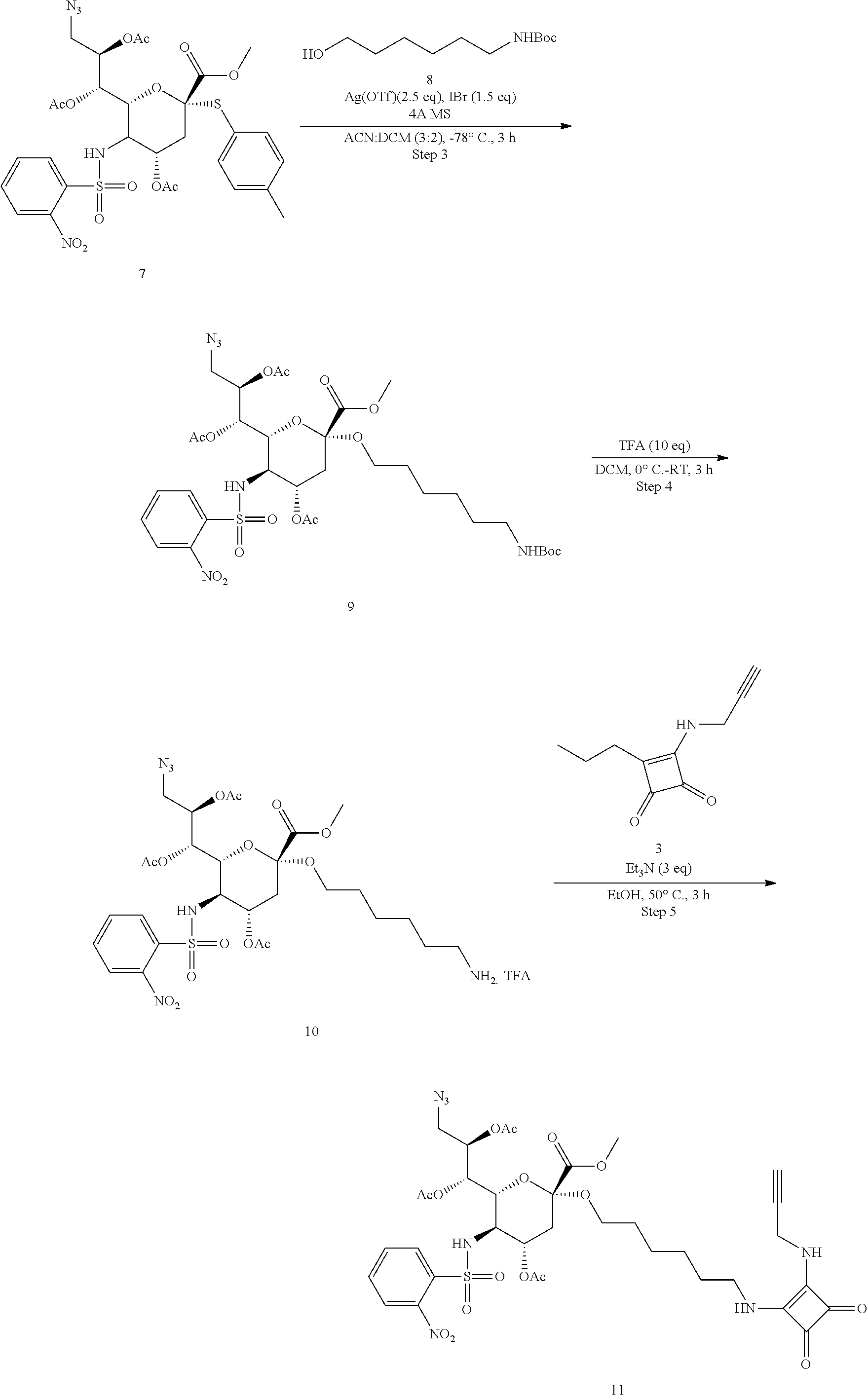

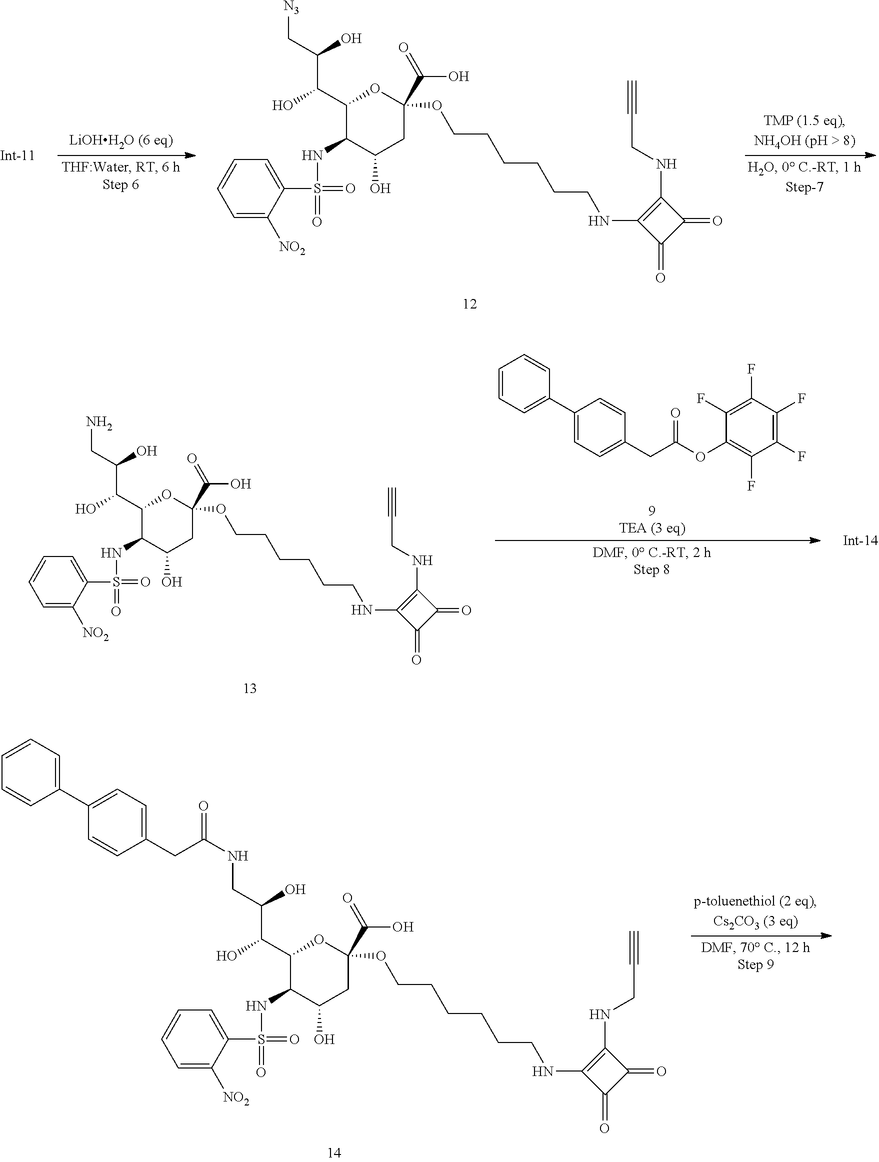 [SMILES: C#CCNc1c(NCCCCCCO[C@]2(C(=O)O)C[C@H](O)[C@@H](NS(=O)(=O)c3ccccc3[N+](=O)[O-])[C@H]([C@H](O)[C@H](O)CN=[N+]=[N-])O2)c(=O)c1=O ; C#CCNc1c(NCCCCCCO[C@]2(C(=O)O)C[C@H](O)[C@@H](NS(=O)(=O)c3ccccc3[N+](=O)[O-])[C@H]([C@H](O)[C@H](O)CN)O2)c(=O)c1=O ; O=C(Cc1ccc(-c2ccccc2)cc1)Oc1c(F)c(F)c(F)c(F)c1F ; C#CCNc1c(NCCCCCCO[C@]2(C(=O)O)C[C@H](O)[C@@H](NS(=O)(=O)c3ccccc3[N+](=O)[O-])[C@H]([C@H](O)[C@H](O)CNC(=O)Cc3ccc(-c4ccccc4)cc3)O2)c(=O)c1=O]

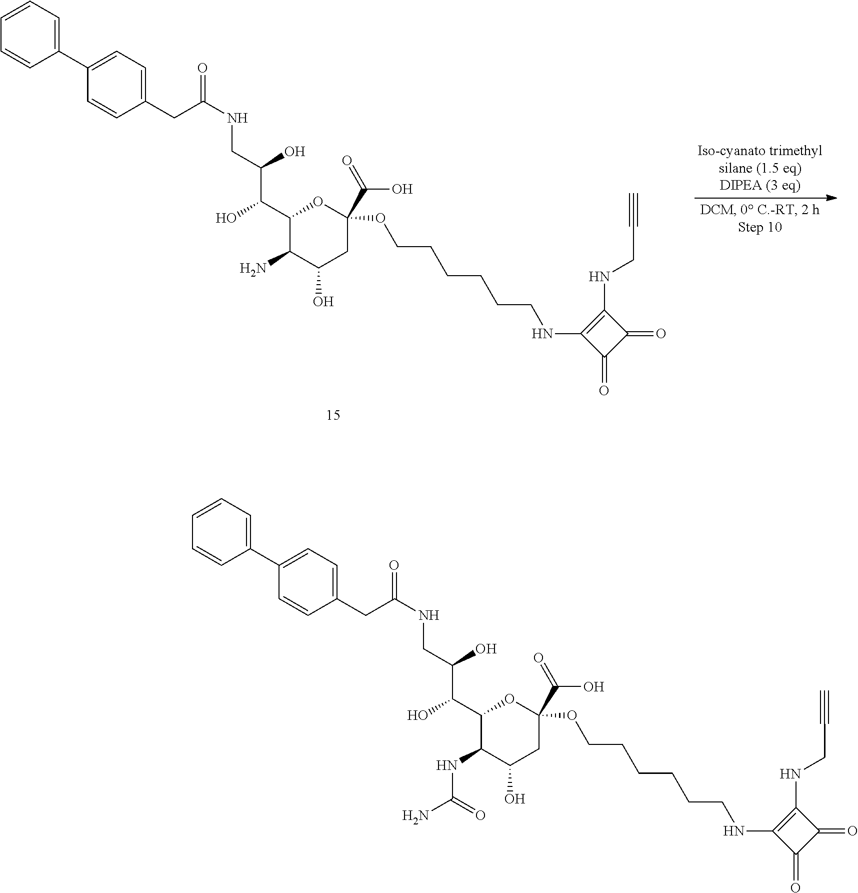 [SMILES: C#CCNc1c(NCCCCCCO[C@]2(C(=O)O)C[C@H](O)[C@@H](N)[C@H]([C@H](O)[C@H](O)CNC(=O)Cc3ccc(-c4ccccc4)cc3)O2)c(=O)c1=O ; C#CCNc1c(NCCCCCCO[C@]2(C(=O)O)C[C@H](O)[C@@H](NC(N)=O)[C@H]([C@H](O)[C@H](O)CNC(=O)Cc3ccc(-c4ccccc4)cc3)O2)c(=O)c1=O]

### Synthesis of 3-ethoxy-4-(prop-2-yn-1-ylamino) cyclobut-3-ene-1,2-dione (3)

To a stirred solution of 3,4-diethoxycyclobut-3-ene-1,2-dione (1) (3 g, 17.63 mmol) in ethanol (30 mL) was added prop-2-yn-1-amine (2) (971 mg, 17.63 mmol) at 0° C. followed by addition of triethylamine (7.37 mL, 52.89 mmol). The resulting reaction mixture was stirred at room temperature for 4 h. After completion, the reaction mixture was concentrated under reduced pressure and the crude residue obtained was purified by silica-gel column chromatography using 50% ethyl acetate: hexane to afford the desired product 3-ethoxy-4-(prop-2-yn-1-ylamino) cyclobut-3-ene-1,2-dione (3) as a yellow solid, Yield: 2.5 g, 79%; LCMS (ESI+) m/z 180.1 [M+H]⁺.

### Synthesis of methyl (2R,4S,5R,6R)-5-amino-6-((R,2R)-3-azido-1,2-dihydroxypropyl)-4-hydroxy-2-(p-tolylthio) tetrahydro-2H-pyran-2-carboxylate (5)

To a stirred solution of methyl (2R,4S,5R,6R)-5-acetamido-6-((1R,2R)-3-azido-1,2-dihydroxypropyl)-4-hydroxy-2-(p-tolylthio) tetrahydro-2H-pyran-2-carboxylate (4) (5 g, 11 mmol) in methanol (150 mL) was added methane sulfonic acid (3.57 mL, 55.01 mmol) dropwise at 0° C., then the reaction mixture was heated at 80° C. for 16 h. After completion, the reaction mixture was quenched by addition of triethylamine at 0° C. maintaining neutral pH. The reaction mixture was concentrated under reduced pressure to afford of the desired product methyl (2R,4S,5R,6R)-5-amino-6-((1R,2R)-3-azido-1,2-dihydroxypropyl)-4-hydroxy-2-(p-tolylthio) tetrahydro-2H-pyran-2-carboxylate (5), which was used as such for the next step, Yield: 4 g, 88%; LCMS (ESI+) m/z 413.4 [M+H]⁺

### Synthesis of (1R,2R)-1-((2R,3R,4S,6R)-4-acetoxy-6-(methoxycarbonyl)-3-((2-nitrophenyl) sulfonamido)-6-(p-tolylthio) tetrahydro-2H-pyran-2-yl)-3-azidopropane-1,2-diyl diacetate (7)

To a stirred solution of methyl (2R,4S,5R,6R)-5-amino-6-((1R,2R)-3-azido-1,2-dihydroxy propyl)-4-hydroxy-2-(p-tolylthio) tetrahydro-2H-pyran-2-carboxylate (5) (4 g, 9.7 mmol) in N,N-dimethylformamide (40 ml) was added triethylamine (4 ml, 29.09 mmol) dropwise at 0° C. After 15 min of stirring, 2-nitrobenzenesulfonyl chloride (6) (2.79 g, 12.61 mmol) dissolved in N,N-dimethylformamide was added dropwise at 0° C. The reaction mixture was stirred at rt for 2 h. After completion, 4-dimethyl aminopyridine (1.18 g, 9.70 mmol) and acetic anhydride (9.17 ml, 96.98 mmol) were added dropwise at 0° C. over 10 min and the reaction mixture was stirred at room temperature for 12 h. After completion, the reaction mixture was diluted with cold water, extracted with ethyl acetate, and the organic layer was washed twice with ice cold water (250 mL), dried over anhydrous sodium sulfate, filtered and concentrated under reduced pressure. The crude residue obtained was purified by C18 reverse phase column chromatography using acetonitrile and water (+0.05% TFA) as eluents to afford the desired product (1R,2R)-1-((2R,3R,4S,6R)-4-acetoxy-6-(methoxycarbonyl)-3-((2-nitrophenyl)sulfonamido)-6-(p-tolylthio)tetrahydro-2H-pyran-2-yl)-3-azidopropane-1,2-diyl diacetate (7) as off white solid, Yield: 2.5 g, 35%; LCMS (ESI+) m/z 741.4 [M+H2O]⁺.

### Synthesis of (1R,2R)-1-((2R,3R,4S,6R)-4-acetoxy-6-((6-((tert-butoxy carbonyl)amino)hexyl) oxy)-6-(methoxycarbonyl)-3-((2-nitrophenyl) sulfonamido) tetrahydro-2H-pyran-2-yl)-3-azidopropane-1,2-diyl diacetate (9)

A solution (1R,2R)-1-((2R,3R,4S,6S)-4-acetoxy-6-(methoxycarbonyl)-3-((2-nitrophenyl)sulfonamido)-6-(p-tolylthio)tetrahydro-2H-pyran-2-yl)-3-azidopropane-1,2-diyl diacetate (7) (2 g, 2.76 mmol) and tert-butyl (6-hydroxyhexyl)carbamate (8) (720 mg, 3.32 mmol), Silver (1+) trifluoro methane sulfonate (AgOTf) (1.78 g, 6.91 mmol) and activated 4 Å powdered molecular sieves (5 g) in anhydrous dichloromethane (12 mL) and anhydrous acetonitrile (24 mL) was stirred at room temperature for 1 h under nitrogen. The solution was cooled to −78° C., iodine monobromide (857 mg, 4.15 mmol) solution in dichloromethane (4 mL) was added dropwise, and the resulting mixture was stirred at same temperature for 2 h. After completion, the reaction mixture was quenched by addition of triethylamine (5 mL) and warmed to room temperature. The reaction mixture was filtered, the filtrate was washed with a saturated aqueous solution of sodium thiosulfate, dried over sodium sulfate, filtered and concentrated under reduced pressure. The crude residue obtained was purified by C18 reverse phase column chromatography using water: acetonitrile as eluents to afford the desired product (1R,2R)-1-((2R,3R,4S,6R)-4-acetoxy-6-((6-((tert-butoxy carbonyl)amino)hexyl)oxy)-6-(methoxy carbonyl)-3-((2-nitrophenyl) sulfonamido) tetrahydro-2H-pyran-2-yl)-3-azidopropane-1,2-diyl diacetate (9) as a pale yellow solid, Yield: 1 g, 44%; LCMS (ESI−) m/z 815.7 [M−H]⁻.

### Synthesis of (1R,2R)-1-((2R,3R,4S,6R)-4-acetoxy-6-((6-aminohexyl)oxy)-6-(methoxycarbonyl)-3-((2-nitrophenyl) sulfonamido) tetrahydro-2H-pyran-2-yl)-3-azidopropane-1,2-diyl diacetate TFA salt (10)

To a stirred solution of (1R,2R)-1-((2R,3R,4S,6R)-4-acetoxy-6-((6-((tert-butoxy carbonyl)amino)hexyl)oxy)-6-(methoxycarbonyl)-3-((2-nitrophenyl)sulfonamido)tetrahydro-2H-pyran-2-yl)-3-azidopropane-1,2-diyl diacetate (9) (1 g, 1.22 mmol) in anhydrous dichloromethane (10 mL) was added trifluoro acetic acid (0.9 mL) dropwise at 0° C. and the reaction mixture was stirred at room temperature for 3 h. After completion, the solvent was concentrated to afford the desired product (1R,2R)-1-((2R,3R,4S,6R)-4-acetoxy-6-((6-aminohexyl)oxy)-6-(methoxycarbonyl)-3-((2-nitrophenyl) sulfonamido) tetrahydro-2H-pyran-2-yl)-3-azidopropane-1,2-diyl diacetate TFA salt (10) as a light-yellow sticky syrup. The crude residue was directly used for the next step without further purification, Yield: 800 mg, Crude; LCMS (ESI+) m/z 717.5 [M+H]⁺.

### Synthesis of (1R,2R)-1-((2R,3R,4S,6R)-4-acetoxy-6-((6-((3,4-dioxo-2-(prop-2-yn-1-ylamino) cyclobut-1-en-1-yl)amino)hexyl)oxy)-6-(methoxycarbonyl)-3-((2-nitrophenyl) sulfonamido) tetrahydro-2H-pyran-2-yl)-3-azidopropane-1,2-diyl diacetate (11)

To a stirred solution of (1R,2R)-1-((2R,3R,4S,6R)-4-acetoxy-6-((6-aminohexyl)oxy)-6-(methoxycarbonyl)-3-((2-nitrophenyl)sulfonamido)tetrahydro-2H-pyran-2-yl)-3-azidopropane-1,2-diyl diacetate TFA salt (10) (0.8 g, 0.98 mmol) in ethanol (5 mL), was added triethylamine (0.55 mL, 3.93 mmol). After 10 minutes 3-ethoxy-4-(prop-2-yn-1-ylamino) cyclobut-3-ene-1,2-dione (3), 0.18 g, 0.98 mmol) was added at room temperature, then the resulting reaction mixture was stirred at 50° C. for 3 h. After completion, the reaction mixture was concentrated under reduced pressure and the crude residue obtained was purified by C18 reverse phase column chromatography using 0.05% TFA: CH₃CN as eluent to afford the desired product (1R,2R)-1-((2R,3R,4S,6R)-4-acetoxy-6-((6-((3,4-dioxo-2-(prop-2-yn-1-ylamino) cyclobut-1-en-1-yl)amino)hexyl)oxy)-6-(methoxycarbonyl)-3-((2-nitrophenyl) sulfonamido)tetrahydro-2H-pyran-2-yl)-3-azidopropane-1,2-diyl diacetate (11) as yellow solid, Yield: 500 mg, 60%; LCMS (ESI+) m/z 850.8 [M+H]⁺.

### Synthesis of (2R,4S,5R,6R)-6-((1R,2R)-3-azido-1,2-dihydroxypropyl)-2-((6-((3,4-dioxo-2-(prop-2-yn-1-ylamino) cyclobut-1-en-1-yl)amino)hexyl)oxy)-4-hydroxy-5-((2-nitrophenyl) sulfonamido) tetrahydro-2H-pyran-2-carboxylic acid (12)

To a stirred solution (1R,2R)-1-((2R,3R,4S,6R)-4-acetoxy-6-((6-((3,4-dioxo-2-(prop-2-yn-1-ylamino)cyclobut-1-en-1-yl)amino)hexyl)oxy)-6-(methoxycarbonyl)-3-((2-nitrophenyl) sulfon amido)tetrahydro-2H-pyran-2-yl)-3-azidopropane-1,2-diyl diacetate (11) (0.5 g, 0.59 mmol) in tetrahydrofuran (5 mL) was added a solution of lithium hydroxide monohydrate (148 mg, 3.53 mmol) in water (2 mL). The reaction mixture was stirred at room temperature for 6 h. After completion, the reaction mixture was treated with acidic (Dowex 50, H⁺) to pH˜6 and the suspension was filtered through sintered funnel with methanol washing. The filtrate was concentrated under reduced pressure to get crude. The crude material (2R,4S,5R,6R)-6-((1R,2R)-3-azido-1,2-dihydroxypropyl)-2-((6-((3,4-dioxo-2-(prop-2-yn-1-ylamino)cyclobut-1-en-1-yl)amino)hexyl)oxy)-4-hydroxy-5-((2-nitrophenyl)sulfon-amido)tetrahydro-2H-pyran-2-carboxylic acid (12) used as such without further purification, Yield: 400 mg, Crude; LCMS (ESI+) m/z 710.4 [M+H]⁺.

### Synthesis of (2R,4S,5R,6R)-6-((1R,2R)-3-amino-1,2-dihydroxypropyl)-2-((6-((3,4-dioxo-2-(prop-2-yn-1-ylamino) cyclobut-1-en-1-yl)amino)hexyl)oxy)-4-hydroxy-5-((2-nitrophenyl) sulfonamido) tetrahydro-2H-pyran-2-carboxylic acid (13)

To a stirred solution of (2R,4S,5R,6R)-6-((1R,2R)-3-azido-1,2-dihydroxypropyl)-2-((6-((3,4-dioxo-2-(prop-2-yn-1-ylamino)cyclobut-1-en-1-yl)amino)hexyl)oxy)-4-hydroxy-5-((2-nitro phenyl)sulfonamido)tetrahydro-2H-pyran-2-carboxylic acid (12) (0.4 g, 0.56 mmol) in H₂O (6 mL), NH₄OH (0.1 mL, pH>8) was added TMP (0.845 mL, 1.5 eq, 1M soln in THF) at 0° C. and the reaction mixture was stirred for 1 h at the same temperature. After completion, the reaction mixture was washed with ethyl acetate, and the aqueous phase was dried by lyophilization to afford the desired product (2R,4S,5R,6R)-6-((1R,2R)-3-amino-1,2-dihydroxypropyl)-2-((6-((3,4-dioxo-2-(prop-2-yn-1-ylamino)cyclobut-1-en-1-yl)amino)hexyl)oxy)-4-hydroxy-5-((2-nitro phenyl)sulfonamido)tetrahydro-2H-pyran-2-carboxylic acid (13) as a white solid, which was directly used in the next step without further purification, Yield: 350 mg, Crude; LCMS (ESI+) m/z 684.4 [M+H]⁺

### Synthesis of (2R,4S,5R,6R)-6-((1R,2R)-3-(2-([1,1′-biphenyl]-4-yl)acetamido)-1,2-dihydroxy propyl)-2-((6-((3,4-dioxo-2-(prop-2-yn-1-ylamino)cyclobut-1-en-1-yl)amino)hexyl)oxy)-4-hydroxy-5-((2-nitrophenyl)sulfonamido)tetrahydro-2H-pyran-2-carboxylic acid (14)

To a stirred solution of (2R,4S,5R,6R)-6-((1R,2R)-3-amino-1,2-dihydroxypropyl)-2-((6-((3,4-dioxo-2-(prop-2-yn-1-ylamino)cyclobut-1-en-1-yl)amino)hexyl)oxy)-4-hydroxy-5-((2-nitro phenyl)sulfonamido)tetrahydro-2H-pyran-2-carboxylic acid (13) (350 mg, 0.511 mmol) in DMF (2 mL) was added penta-fluorophenyl 2-([1,1′-biphenyl]-4-yl)acetate (9) (213 mg, 0.56 mmol, 1.1 eq) at room temperature. After 10 minutes, triethylamine (0.21 mL, 1.54 mmol) was added dropwise at 0° C. The resulting reaction mixture was stirred at room temperature for 2 h. After completion the reaction mixture was purified by C18 reverse phase column chromatography to afford the desired product (2R,4S,5R,6R)-6-((1R,2R)-3-(2-([1,1′-biphenyl]-4-yl)acetamido)-1,2-dihydroxypropyl)-2-((6-((3,4-dioxo-2-(prop-2-yn-1-ylamino)cyclobut-1-en-1-yl)amino)hexyl)oxy)-4-hydroxy-5-((2-nitrophenyl)sulfonamido) tetrahydro-2H-pyran-2-carboxylic acid (14) as white solid, Yield:70 mg, 16%; LCMS (ESI+) m/z 878.8 [M+H]⁺

### Synthesis of (2R,4S,5R,6R)-6-((1R,2R)-3-(2-([1,1′-biphenyl]-4-yl) acetamido)-1,2-dihydroxy propyl)-5-amino-2-((6-((3,4-dioxo-2-(prop-2-yn-1-ylamino) cyclobut-1-en-1-yl)amino)hexyl) oxy)-4-hydroxy tetrahydro-2H-pyran-2-carboxylic acid (15)

To a stirred solution of (2R,4S,5R,6R)-6-((1R,2R)-3-(2-([1,1′-biphenyl]-4-yl)acetamido)-1,2-dihydroxypropyl)-2-((6-((3,4-dioxo-2-(prop-2-yn-1-ylamino)cyclobut-1-en-1-yl)amino)hexyl)oxy)-4-hydroxy-5-((2-nitrophenyl)sulfonamido)tetrahydro-2H-pyran-2-carboxylic acid (14), (70 mg, 0.079 mmol) in anhydrous N,N-dimethylformamide (1 mL), 4-methylbenzenethiol (20 mg, 0.159 mmol) and cesium carbonate (83 mg, 0.255 mmol) were added at 0° C. The reaction mixture was stirred at 70° C. for 12 h. After completion, crude reaction mixture was purified by C18 reverse phase chromatography using water and acetonitrile (+0.05% TFA) as eluents to afford the desired product (2R,4S,5R,6R)-6-((1R,2R)-3-(2-([1,1′-biphenyl]-4-yl)acetamido)-1,2-dihydroxy propyl)-5-amino-2-((6-((3,4-dioxo-2-(prop-2-yn-1-ylamino)cyclobut-1-en-1-yl)amino)hexyl)oxy)-4-hydroxy tetrahydro-2H-pyran-2-carboxylic acid (15) as off white solid, Yield: 15 mg, 27%; LCMS (ESI+) m/z 693.1 [M+H]⁺.

### Synthesis of (2R,4S,5R,6R)-6-((1R,2R)-3-(2-([1,1′-biphenyl]-4-yl) acetamido)-1,2-dihydroxy propyl)-2-((6-((3,4-dioxo-2-(prop-2-yn-1-ylamino) cyclobut-1-en-1-yl)amino)hexyl)oxy)-4-hydroxy-5-ureidotetrahydro-2H-pyran-2-carboxylic acid (224)

To a stirred solution of (2R,4S,5R,6R)-6-((1R,2R)-3-(2-([1,1′-biphenyl]-4-yl)acetamido)-1,2-dihydroxypropyl)-5-amino-2-((6-((3,4-dioxo-2-(prop-2-yn-1-ylamino)cyclobut-1-en-1-yl)amino)hexyl)oxy)-4-hydroxytetrahydro-2H-pyran-2-carboxylic acid (15) (15 mg, 0.02 mmol) in anhydrous dichloromethane (1 mL) was added isocyanato trimethyl silane (3.74 mg, 0.06 mmol) in dichloromethane (0.1 ml) at 0° C. followed by addition of N,N-diisopropylethylamine (0.01 mL, 0.06 mmol) and the reaction mixture was stirred at room temperature for 2 h. After completion the reaction mixture was concentrated under reduced pressure and the crude residue obtained was purified by C18 preparative hplc using water and acetonitrile (+0.05% TFA) as eluents to afford the desired product (2R,4S,5R,6R)-6-((1R,2R)-3-(2-([1,1′-biphenyl]-4-yl)acetamido)-1,2-dihydroxypropyl)-2-((6-((3,4-dioxo-2-(prop-2-yn-1-ylamino) cyclobut-1-en-1-yl)amino)hexyl)oxy)-4-hydroxy-5-ureidotetrahydro-2H-pyran-2-carboxylic acid (224) as a white solid, Yield: 4.45 mg, 28%; LCMS (ESI+) m/z 736.8 [M+H]⁺.

¹H NMR (401 MHz, MeOD) δ 7.59 (ddt, J=10.4, 8.3, 1.8 Hz, 4H), 7.47-7.37 (m, 4H), 7.35-7.28 (m, 1H), 4.43 (s, 2H), 3.93 (s, 1H), 3.80-3.71 (m, 2H), 3.66-3.56 (m, 4H), 3.50 (d, J=16.5 Hz, 3H), 3.21 (dd, J=13.5, 8.4 Hz, 1H), 2.85-2.73 (m, 2H), 1.65 (t, J=11.9 Hz, 2H), 1.55 (s, 4H), 1.33 (d, J=15.4 Hz, 4H), 0.9 (s, 1H).

### (2R,4S,5R,6R)-6-((1R,2R)-3-(2-([1,1′-biphenyl]-4-yl) acetamido)-1,2-dihydroxypropyl)-2-((2,18-dioxo-6,9,12,15,22,25-hexaoxa-3,19-diazaheptacosan-27-yl) oxy)-4-hydroxy-5-(2-hydroxyacetamido) tetrahydro-2H-pyran-2-carboxylic acid (Cpd. 236)

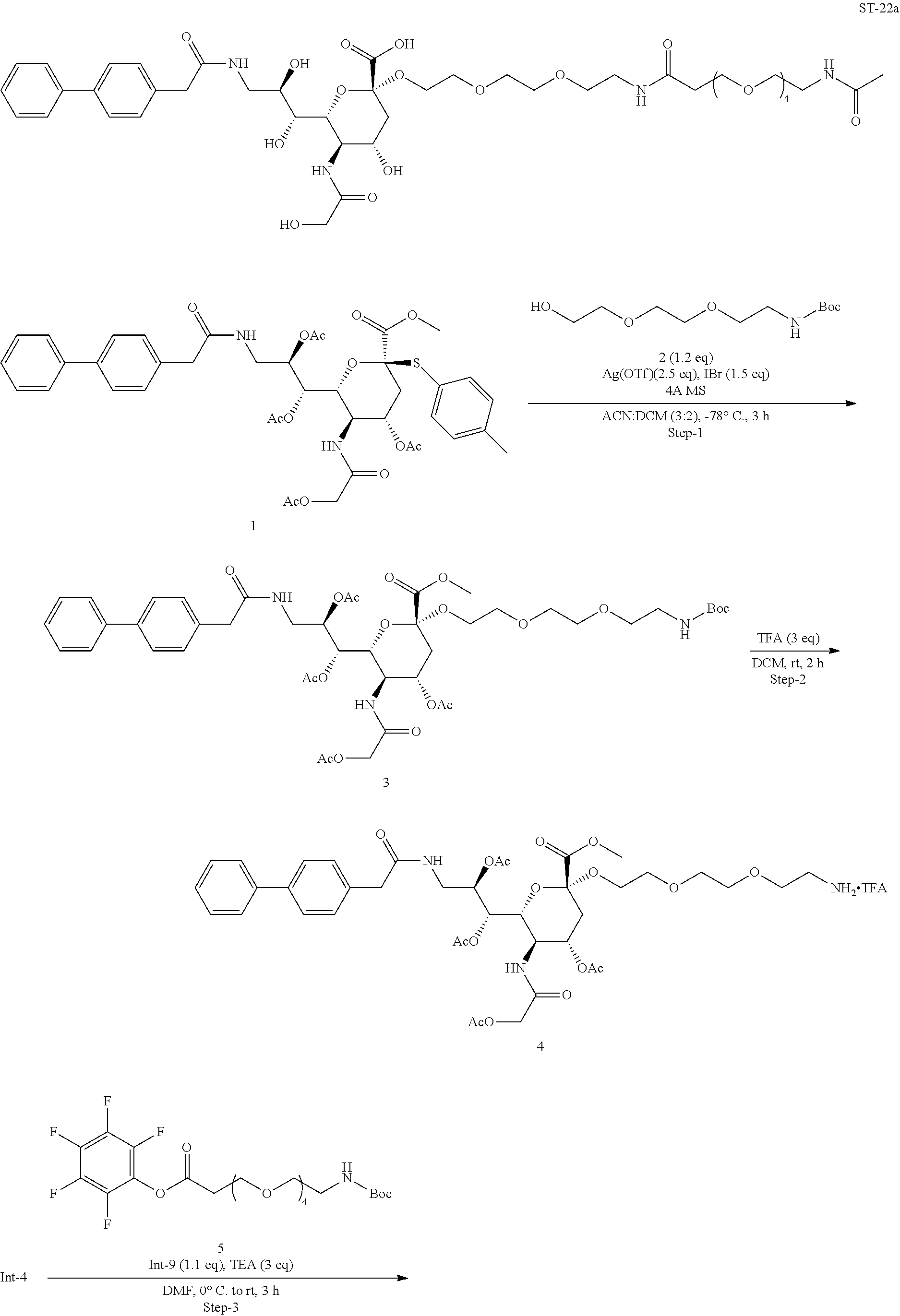

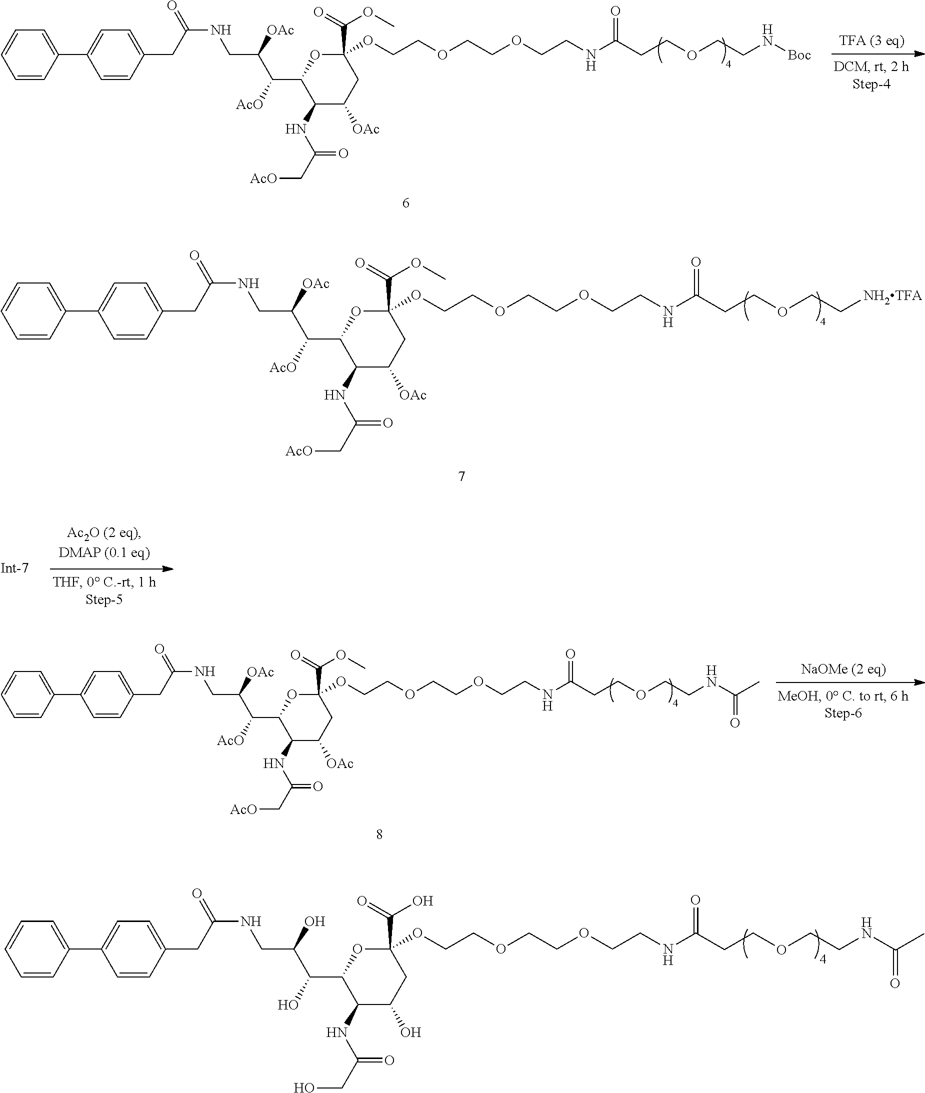

### Synthesis of (1R,2R)-3-(2-([1,1′-biphenyl]-4-yl) acetamido)-1-((2R,3R,4S,6R)-4-acetoxy-3-(2-acetoxyacetamido)-6-((2,2-dimethyl-4-oxo-3,8,11-trioxa-5-azatridecan-13-yl) oxy)-6-(methoxycarbonyl) tetrahydro-2H-pyran-2-yl) propane-1,2-diyl diacetate (3)

To a stirred solution of (1R,2R)-3-(2-([1,1′-biphenyl]-4-yl)acetamido)-1-((2R,3R,4S,6R)-4-acetoxy-3-(2-acetoxyacetamido)-6-(methoxycarbonyl)-6-(p-tolylthio)tetrahydro-2H-pyran-2-yl)propane-1,2-diyl diacetate (1A) (1 g, 1.24 mmol) and tert-butyl (2-(2-(2-hydroxyethoxy)ethoxy)ethyl)carbamate (2) (463 g, 1.86 mmol) in anhydrous dichloromethane (6 mL) and anhydrous acetonitrile (24 mL) were added silver(1+) trifluoro-methane sulfonate (490 mg, 3.10 mmol) and activated 4 Å powdered molecular sieves (2 g). The reaction mixture was stirred at room temperature for 1 h under nitrogen atmosphere, cooled to −78° C. then a solution of iodine monobromide (383 mg, 1.86 mmol) in dichloromethane (2 mL) was added dropwise. After complete addition the reaction mixture was stirred at the same temperature for 2 h. After completion, the reaction mixture was quenched by addition of triethylamine (1 mL), filtered and the filtrate was washed with a saturated solution of sodium thiosulfate, dried over sodium sulfate, filtered, and concentrated. The crude residue was purified by silica gel column chromatography using ethyl acetate in Hexane as eluents to afford the desired product (1R,2R)-3-(2-([1,1′-biphenyl]-4-yl) acetamido)-1-((2R,3R,4S,6R)-4-acetoxy-3-(2-acetoxyacetamido)-6-((2,2-dimethyl-4-oxo-3,8,11-trioxa-5-azatridecan-13-yl) oxy)-6-(methoxycarbonyl) tetrahydro-2H-pyran-2-yl) propane-1,2-diyl diacetate (3) as colorless sticky liquid. Yield: 371 mg, 32%; LCMS (ESI±) m/z 932.8 [M+H]⁺.

### Synthesis of (1R,2R)-3-(2-([1,1′-biphenyl]-4-yl) acetamido)-1-((2R,3R,4S,6R)-4-acetoxy-3-(2-acetoxyacetamido)-6-(methoxycarbonyl)-6-(2-(2-(2-((2,2,2-trifluoroacetyl)-14-azaneyl) ethoxy) ethoxy) ethoxy) tetrahydro-2H-pyran-2-yl) propane-1,2-diyl diacetate (4)

To a stirred solution of (1R,2R)-3-(2-([1,1′-biphenyl]-4-yl) acetamido)-1-((2R,3R,4S,6R)-4-acetoxy-3-(2-acetoxyacetamido)-6-((2,2-dimethyl-4-oxo-3,8,11-trioxa-5-azatridecan-13-yl) oxy)-6-(methoxycarbonyl) tetrahydro-2H-pyran-2-yl) propane-1,2-diyl diacetate (3) (370 mg, 0.39 mmol) in anhydrous dichloromethane (2 mL), was added trifluoroacetic acid (1 mL) at 0° C. The reaction mixture was stirred at room temperature for 2 h. After completion, the mixture was concentrated and the crude (1R,2R)-3-(2-([1,1′-biphenyl]-4-yl) acetamido)-1-((2R,3R,4S,6R)-4-acetoxy-3-(2-acetoxyacetamido)-6-(methoxycarbonyl)-6-(2-(2-(2-((2,2,2-trifluoroacetyl)-14-azaneyl) ethoxy) ethoxy) ethoxy) tetrahydro-2H-pyran-2-yl) propane-1,2-diyl diacetate (4) was directly used for the next step without further purification. Yield: 350 mg, Crude; LCMS (ESI±) m/z 832.8 [M+H]⁺.

### Synthesis of (1R,2R)-3-(2-([1,1′-biphenyl]-4-yl) acetamido)-1-((2R,3R,4S,6R)-4-acetoxy-3-(2-acetoxyacetamido)-6-((2,2-dimethyl-4,20-dioxo-3,8,11,14,17,24,27-heptaoxa-5,21-diazanonacosan-29-yl) oxy)-6-(methoxycarbonyl) tetrahydro-2H-pyran-2-yl) propane-1,2-diyl diacetate (6)

To a stirred solution of (1R,2R)-1-((2R,3R,4S,6R)-4-acetoxy-3-(2-acetoxyacetamido)-6-(methoxycarbonyl)-6-(2-(2-(2-((2,2,2-trifluoroacetyl)-14-azaneyl)ethoxy)ethoxy)ethoxy)tetrahydro-2H-pyran-2-yl)-3-(2-(4-chlorophenyl)acetamido)propane-1,2-diyl diacetate (4) (350 mg, 0.24 mmol) in N,N-Dimethyl formamide (3 mL) were added triethylamine (0.18 mL, 1.26 mmol), perfluorophenyl 2,2-dimethyl-4-oxo-3,8,11,14,17-pentaoxa-5-azaicosan-20-oate (5) (115 mg, 0.46 mmol) and the reaction mixture was stirred at room temperature for 3 h. After completion the reaction mixture was concentrated and the crude residue was purified by silica gel column methanol and dichloromethane as eluents to afford the desired product (1R,2R)-3-(2-([1,1′-biphenyl]-4-yl) acetamido)-1-((2R,3R,4S,6R)-4-acetoxy-3-(2-acetoxyacetamido)-6-((2,2-dimethyl-4,20-dioxo-3,8,11,14,17,24,27-heptaoxa-5,21-diazanonacosan-29-yl) oxy)-6-(methoxycarbonyl) tetrahydro-2H-pyran-2-yl) propane-1,2-diyl diacetate (6). Yield: 260 mg, 66%; LCMS (ESI±) m/z 1180.1 [M+H]⁺.

### Synthesis of (1R,2R)-3-(2-([1,1′-biphenyl]-4-yl) acetamido)-1-((2R,3R,4S,6R)-4-acetoxy-3-(2-acetoxyacetamido)-6-(methoxycarbonyl)-6-((1,1,1-trifluoro-2,18-dioxo-6,9,12,15,22,25-hexaoxa-314,19-diazaheptacosan-27-yl) oxy) tetrahydro-2H-pyran-2-yl) propane-1,2-diyl diacetate (7)

To a stirred solution of (1R,2R)-3-(2-([1,1′-biphenyl]-4-yl) acetamido)-1-((2R,3R,4S,6R)-4-acetoxy-3-(2-acetoxyacetamido)-6-((2,2-dimethyl-4,20-dioxo-3,8,11,14,17,24,27-heptaoxa-5,21-diazanonacosan-29-yl) oxy)-6-(methoxycarbonyl) tetrahydro-2H-pyran-2-yl) propane-1,2-diyl diacetate (6) (260 mg, 0.22 mmol) in anhydrous dichloromethane (1 mL), was added trifluoroacetic acid (0.5 mL) at 0° C. The reaction mixture was stirred at room temperature for 2 h. After completion, the reaction mixture was concentrated to afford the desired product (1R,2R)-3-(2-([1,1′-biphenyl]-4-yl) acetamido)-1-((2R,3R,4S,6R)-4-acetoxy-3-(2-acetoxyacetamido)-6-(methoxycarbonyl)-6-((1,1,1-trifluoro-2,18-dioxo-6,9,12,15,22,25-hexaoxa-314,19-diazaheptacosan-27-yl) oxy) tetrahydro-2H-pyran-2-yl) propane-1,2-diyl diacetate (7) as yellow solid, which was directly used for the next step without further purification. Yield: 170 mg, Crude; LCMS (ESI±) m/z 1077.9 [M−H]⁻.

### Synthesis of (1R,2R)-3-(2-([1,1′-biphenyl]-4-yl) acetamido)-1-((2R,3R,4S,6R)-4-acetoxy-3-(2-acetoxyacetamido)-6-((2,18-dioxo-6,9,12,15,22,25-hexaoxa-3,19-diazaheptacosan-27-yl) oxy)-6-(methoxycarbonyl) tetrahydro-2H-pyran-2-yl) propane-1,2-diyl diacetate (8)

To a stirred solution (1R,2R)-3-(2-([1,1′-biphenyl]-4-yl)acetamido)-1-((2R,3R,4S,6R)-4-acetoxy-3-(2-acetoxyacetamido)-6-(methoxycarbonyl)-6-((1,1,1-trifluoro-2,18-dioxo-6,9,12,15,22,25-hexaoxa-314,19-diazaheptacosan-27-yl)oxy)tetrahydro-2H-pyran-2-yl)propane-1,2-diyl diacetate (7) (160 mg, 0.14 mmol) in tetrahydrofuran (2 mL) where added N,N-Dimethyl amino pyridine (1.6 mg, 0.014 mmol) and acetic anhydride (27 mg, 0.27 mmol) at 0° C. and the reaction mixture was stirred at room temperature for 1 h. After completion, the reaction mixture was partitioned between ethyl acetate and water, the organic layer was separated, dried over anhydrous sodium sulfate, filtered, and concentrated. The crude residue (1R,2R)-3-(2-([1,1′-biphenyl]-4-yl) acetamido)-1-((2R,3R,4S,6R)-4-acetoxy-3-(2-acetoxyacetamido)-6-((2,18-dioxo-6,9,12,15,22,25-hexaoxa-3,19-diazaheptacosan-27-yl) oxy)-6-(methoxycarbonyl) tetrahydro-2H-pyran-2-yl) propane-1,2-diyl diacetate (8) was used as such for the next step. Yield: 80 mg, Crude; LCMS (ESI) m/z 1122.0 [M+H]⁺.

### Synthesis of (2R,4S,5R,6R)-6-((1R,2R)-3-(2-([1,1′-biphenyl]-4-yl) acetamido)-1,2-dihydroxypropyl)-2-((2,18-dioxo-6,9,12,15,22,25-hexaoxa-3,19-diazaheptacosan-27-yl) oxy)-4-hydroxy-5-(2-hydroxyacetamido) tetrahydro-2H-pyran-2-carboxylic acid (236)

To a stirred solution of (1R,2R)-3-(2-([1,1′-biphenyl]-4-yl) acetamido)-1-((2R,3R,4S,6R)-4-acetoxy-3-(2-acetoxyacetamido)-6-((2,18-dioxo-6,9,12,15,22,25-hexaoxa-3,19-diazaheptacosan-27-yl) oxy)-6-(methoxycarbonyl) tetrahydro-2H-pyran-2-yl) propane-1,2-diyl diacetate (8) (60 mg, 0.053 mmol) in methanol (2 mL) was added sodium methoxide (25% solution in methanol) (13.5 mg, 0.32 mmol) at 0° C. The reaction mixture was stirred at room temperature for 6 h. After completion, the reaction mixture was treated with acidic resin (Dowex 50, H⁺) to pH˜6 and the suspension was filtered, and the filtrate was concentrated. The crude residue was purified by C18 prep-HPLC using acetonitrile and water (+0.05% TFA) as eluents to afford the desired product (2R,4S,5R,6R)-6-((1R,2R)-3-(2-([1,1′-biphenyl]-4-yl) acetamido)-1,2-dihydroxypropyl)-2-((2,18-dioxo-6,9,12,15,22,25-hexaoxa-3,19-diazaheptacosan-27-yl) oxy)-4-hydroxy-5-(2-hydroxyacetamido) tetrahydro-2H-pyran-2-carboxylic acid (236) as white solid. Yield: 25 mg, 50%; LCMS (ESI±) m/z 939.0 [M+H]⁺. ¹H NMR (400 MHz, MeOD): δ 7.66-7.56 (m, 4H), 7.48-7.38 (m, 4H), 7.34 (td, J=7.2, 1.3 Hz, 1H), 4.03 (s, 2H), 3.96-3.79 (m, 4H), 3.78-3.70 (m, 4H), 3.70-3.60 (m, 19H), 3.54 (t, J=5.5 Hz, 4H), 3.44-3.35 (m, 5H), 3.27 (dd, J=13.7, 7.8 Hz, 1H), 2.74 (dd, J=12.7, 4.1 Hz, 1H), 2.47 (t, J=6.2 Hz, 2H), 1.96 (s, 3H), 1.77 (t, J=11.9 Hz, 1H).

### (2R,4S,5R,6R)-6-((1R,2R)-3-(2-([1,1′-biphenyl]-4-yl) acetamido)-1,2-dihydroxypropyl)-2-((28-(2,5-dioxo-2,5-dihydro-1H-pyrrol-1-yl)-10,26-dioxo-3,6,13,16,19,22-hexaoxa-9,25-diazaoctacosyl) oxy)-4-hydroxy-5-(2-hydroxyacetamido) tetrahydro-2H-pyran-2-carboxylic acid (Cpd. 236M)

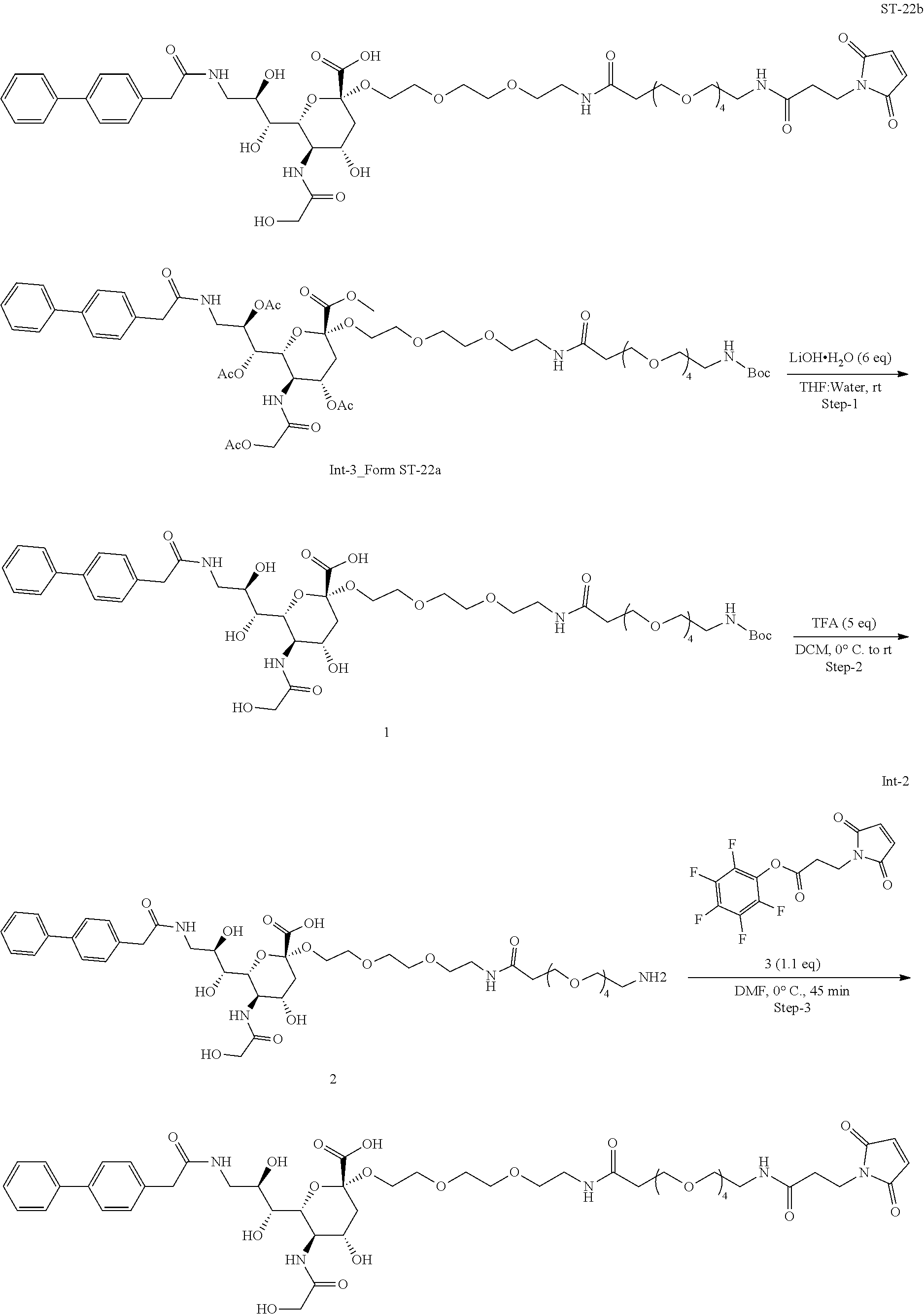

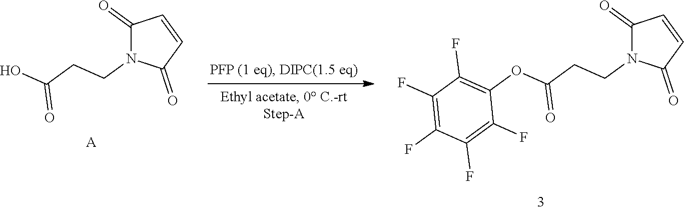

### Synthesis of perfluorophenyl 3-(2,5-dioxo-2,5-dihydro-1H-pyrrol-1-yl) propanoate (3)

To a stirred solution of 3-(2,5-dioxo-2,5-dihydro-1H-pyrrol-1-yl)propanoic acid (A), 2 g, 11.82 mmol) in ethyl acetate (20 mL) was added Di-isopropyl methane di-imine (2.24 g, 17.74 mmol) and 2,3,4,5,6-pentafluorophenol (2.18 g, 11.82 mmol) at 0° C. The reaction mixture was stirred at room temperature for 4 h. After completion, the reaction mixture was concentrated and purified by silica gel column chromatography using ethyl acetate in Hexane as eluents to afford perfluorophenyl 3-(2,5-dioxo-2,5-dihydro-1H-pyrrol-1-yl) propanoate (3) as crystalline solid. Yield: 3 g, 27%. ¹H NMR (400 MHz, DMSO) δ 7.04 (s, 2H), 3.81 (t, J=6.8 Hz, 2H), 3.14 (t, J=6.8 Hz, 2H).

### Synthesis of (2R,4S,5R,6R)-6-((1R,2R)-3-(2-([1,1′-biphenyl]-4-yl) acetamido)-1,2-dihydroxypropyl)-2-((2,2-dimethyl-4,20-dioxo-3,8,11,14,17,24,27-heptaoxa-5,21-diazanonacosan-29-yl) oxy)-4-hydroxy-5-(2-hydroxyacetamido) tetrahydro-2H-pyran-2-carboxylic acid (1)

To a stirred solution (1R,2R)-3-(2-([1,1′-biphenyl]-4-yl) acetamido)-1-((2R,3R,4S,6R)-4-acetoxy-3-(2-acetoxyacetamido)-6-((2,2-dimethyl-4,20-dioxo-3,8,11,14,17,24,27-heptaoxa-5,21-diazanonacosan-29-yl) oxy)-6-(methoxycarbonyl) tetrahydro-2H-pyran-2-yl) propane-1,2-diyl diacetate (Int_3_Form_236) (260 mg, 0.22 mmol) in THF (2 mL) and water (0.8 mL) was added a solution of lithium hydroxide monohydrate (55 mg, 1.32 mmol) in water (2 mL). The reaction mixture was stirred at room temperature for 6 h. After completion, the reaction mixture was treated with acidic resin (Dowex 50, H⁺) to pH˜6, the suspension was filtered and the filtrate was concentrated. The crude residue 2R,4S,5R,6R)-6-((1R,2R)-3-(2-([1,1′-biphenyl]-4-yl) acetamido)-1,2-dihydroxypropyl)-2-((2,2-dimethyl-4,20-dioxo-3,8,11,14,17,24,27-heptaoxa-5,21-diazanonacosan-29-yl) oxy)-4-hydroxy-5-(2-hydroxyacetamido) tetrahydro-2H-pyran-2-carboxylic acid was used as such without further purification. Yield: 21 mg, 96%, LCMS (ESI±) m/z 997.0 [M+H]⁺.

### Synthesis of (2R,4S,5R,6R)-6-((1R,2R)-3-(2-([1,1′-biphenyl]-4-yl) acetamido)-1,2-dihydroxypropyl)-4-hydroxy-5-(2-hydroxyacetamido)-2-((1,1,1-trifluoro-2,18-dioxo-6,9,12,15,22,25-hexaoxa-314,19-diazaheptacosan-27-yl) oxy) tetrahydro-2H-pyran-2-carboxylic acid (2)

To a stirred solution of 2R,4S,5R,6R)-6-((1R,2R)-3-(2-([1,1′-biphenyl]-4-yl) acetamido)-1,2-dihydroxypropyl)-2-((2,2-dimethyl-4,20-dioxo-3,8,11,14,17,24,27-heptaoxa-5,21-diazanonacosan-29-yl) oxy)-4-hydroxy-5-(2-hydroxyacetamido) tetrahydro-2H-pyran-2-carboxylic acid (1) (100 mg, 0.1 mmol) in anhydrous dichloromethane (2 mL), was added trifluoroacetic acid (1 mL) at 0° C. and the reaction mixture was stirred at room temperature for 3 h. After completion, the reaction mixture was concentrated to afford the desired product (2R,4S,5R,6R)-6-((1R,2R)-3-(2-([1,1′-biphenyl]-4-yl) acetamido)-1,2-dihydroxypropyl)-4-hydroxy-5-(2-hydroxyacetamido)-2-((1,1,1-trifluoro-2,18-dioxo-6,9,12,15,22,25-hexaoxa-314,19-diazaheptacosan-27-yl) oxy) tetrahydro-2H-pyran-2-carboxylic acid (2) as a light yellow sticky syrup and the crude was directly used for the next step without further purification. Yield: 120 mg, Crude.

### Synthesis of (2R,4S,5R,6R)-6-((1R,2R)-3-(2-([1,1′-biphenyl]-4-yl) acetamido)-1,2-dihydroxypropyl)-2-((28-(2,5-dioxo-2,5-dihydro-1H-pyrrol-1-yl)-10,26-dioxo-3,6,13,16,19,22-hexaoxa-9,25-diazaoctacosyl) oxy)-4-hydroxy-5-(2-hydroxyacetamido) tetrahydro-2H-pyran-2-carboxylic acid (236M)

To a stirred solution of (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-((1,1,1-trifluoro-2,18-dioxo-6,9,12,15,22,25-hexaoxa-314,19-diazaheptacosan-27-yl)oxy)tetrahydro-2H-pyran-2-carboxylic acid (2) (120 mg, 0.12 mmol) in N,N-Dimethyl formamide (1 mL) were added triethylamine (0.05 mL, 0.48 mmol), perfluorophenyl 3-(2,5-dioxo-2,5-dihydro-1H-pyrrol-1-yl) (44 mg, 0.13 mmol) at 0° C. and the reaction mixture was stirred at 0° C. for 45 min. After completion, the reaction mixture was purified by C18 prep-HPLC using acetonitrile and water (+0.05% TFA) as eluents to afford the desired product (2R,4S,5R,6R)-6-((1R,2R)-3-(2-([1,1′-biphenyl]-4-yl) acetamido)-1,2-dihydroxypropyl)-2-((28-(2,5-dioxo-2,5-dihydro-1H-pyrrol-1-yl)-10,26-dioxo-3,6,13,16,19,22-hexaoxa-9,25-diazaoctacosyl) oxy)-4-hydroxy-5-(2-hydroxyacetamido) tetrahydro-2H-pyran-2-carboxylic acid (236M) as white solid. Yield: 34 mg, 27%, LCMS (ESI±) m/z 1049.1 [M+H]⁺, ¹H NMR (400 MHz, MeOD): δ 7.89 (d, J=7.7 Hz, 1H), 7.61 (dd, J=11.6, 8.3 Hz, 4H), 7.48-7.38 (m, 4H), 7.34 (t, J=7.3 Hz, 1H), 6.83 (s, 2H), 4.03 (s, 2H), 3.96-3.84 (m, 4H), 3.76 (dt, J=18.6, 6.4 Hz, 6H), 3.70-3.58 (m, 22H), 3.53 (dt, J=13.8, 5.3 Hz, 5H), 3.42-3.37 (m, 4H), 2.74 (dd, J=12.8, 3.9 Hz, 1H), 2.47 (td, J=6.6, 3.7 Hz, 4H), 1.78 (t, J=11.7 Hz, 1H).

### (2R,4S,5R,6R)-6-((1R,2R)-3-(2-(4-chlorophenyl) acetamido)-1,2-dihydroxypropyl)-2-((2,18-dioxo-6,9,12,15,22,25-hexaoxa-3,19-diazaheptacosan-27-yl) oxy)-4-hydroxy-5-(2-hydroxyacetamido) tetrahydro-2H-pyran-2-carboxylic acid (Cpd. 237)

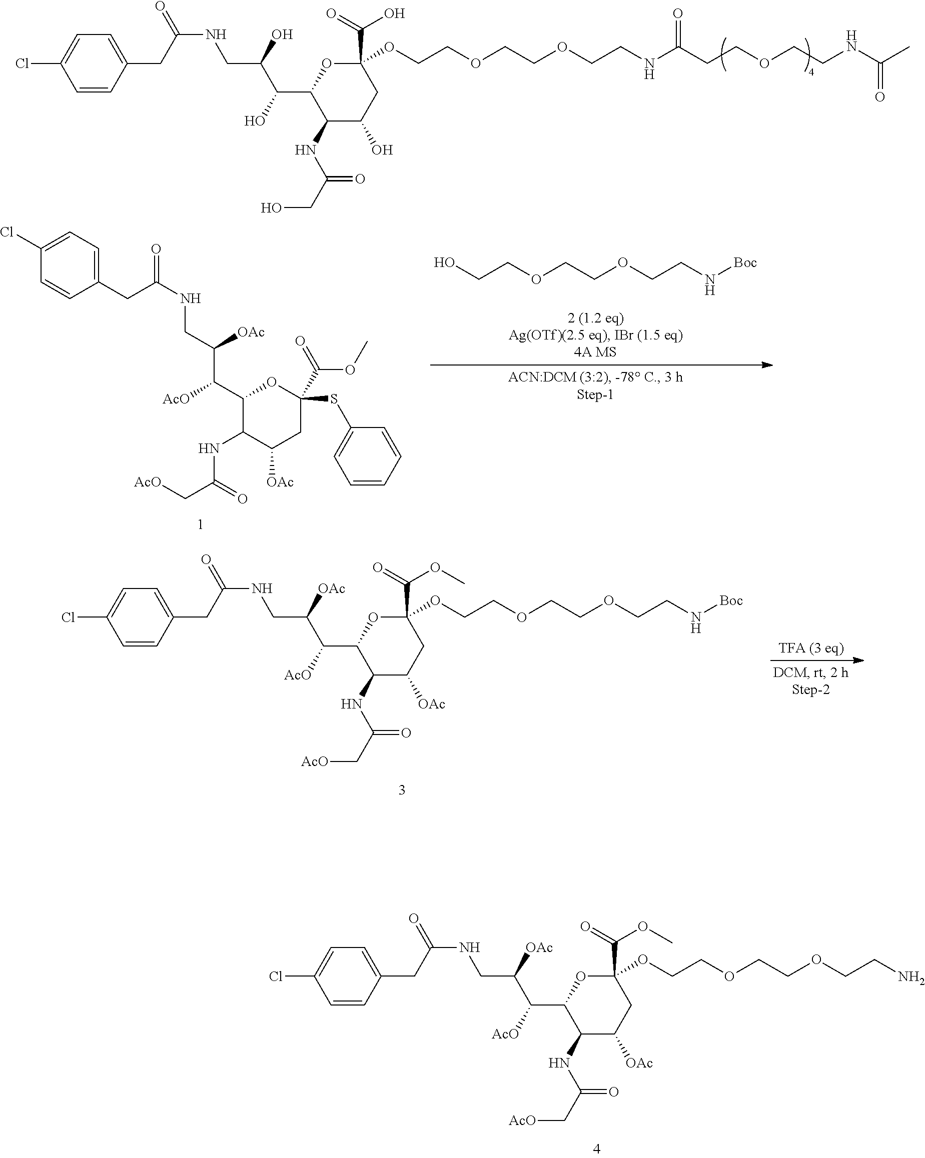

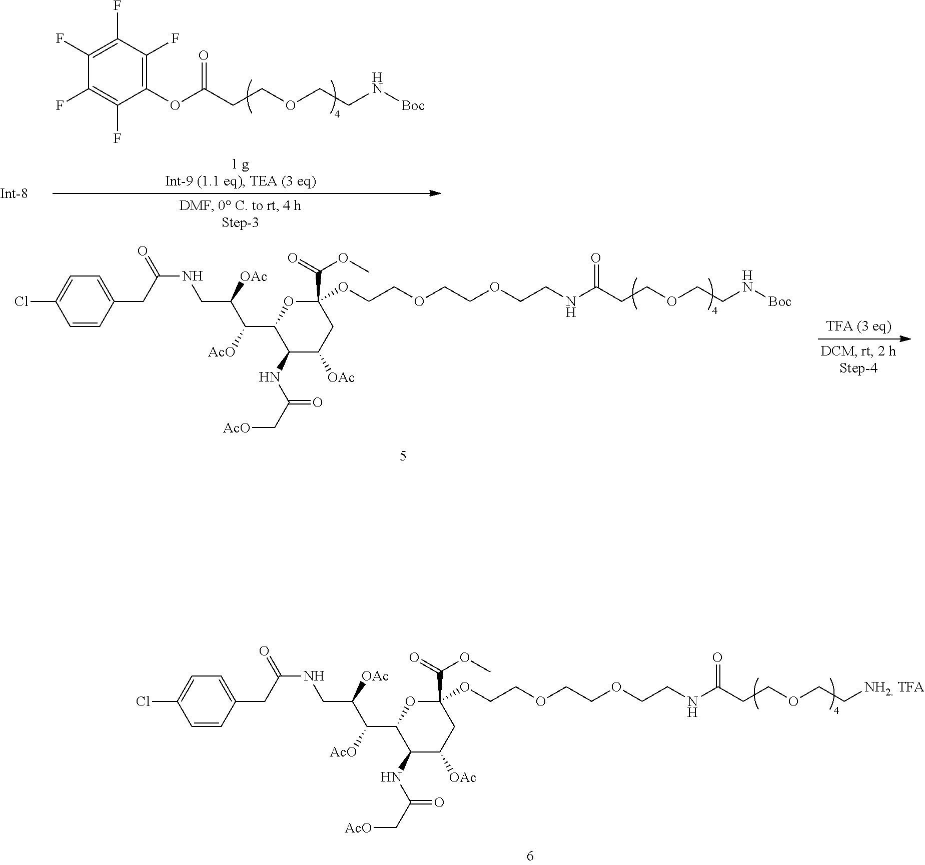

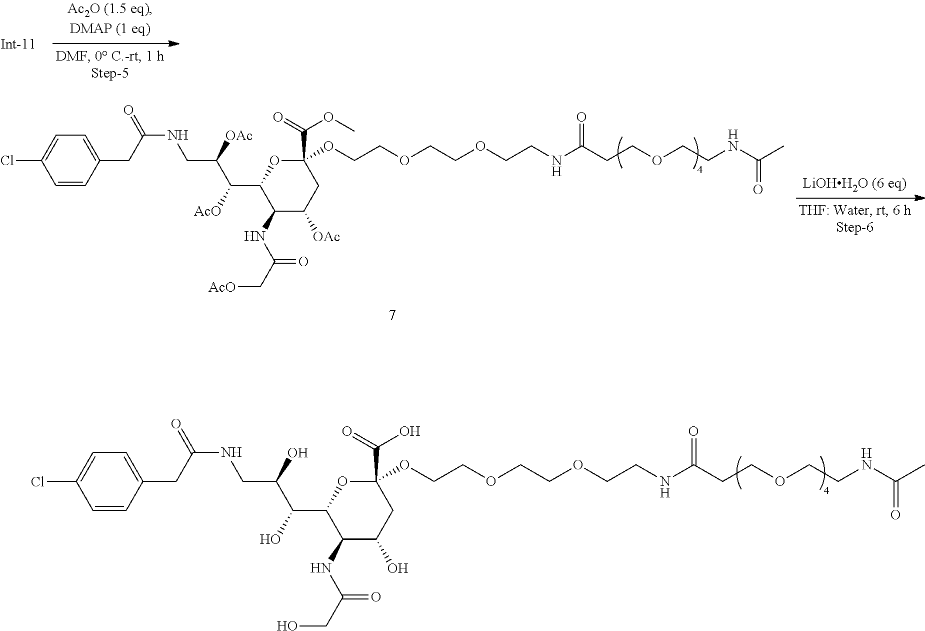

### Synthesis of (1R,2R)-1-((2R,3R,4S,6R)-4-acetoxy-3-(2-acetoxyacetamido)-6-((2,2-dimethyl-4-oxo-3,8,11-trioxa-5-azatridecan-13-yl) oxy)-6-(methoxycarbonyl) tetrahydro-2H-pyran-2-yl)-3-(2-(4-chlorophenyl) acetamido) propane-1,2-diyl diacetate (3)

To stirred solution of ((1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (1) (1 g, 1.31 mmol) and tert-butyl (2-(2-(2-hydroxyethoxy)ethoxy)ethyl)carbamate (2) (390 mg, 1.57 mmol) in anhydrous dichloromethane (6 mL) and anhydrous acetonitrile (24 mL) were added Silver(1+) trifluoro methane sulfonate (837 mg, 3.27 mmol) and activated 4 Å powdered molecular sieves (2 g) and the reaction mixture was stirred at room temperature for 1 h under nitrogen atmosphere. The solution was cooled to −78° C. and the solution of iodine monobromide (403 mg, 1.96 mmol) dissolved in dichloromethane (1 mL) was added dropwise to the reaction mixture, stirred at the same temperature for 2 h. After completion, the reaction mixture was quenched by addition of triethylamine (1 mL). The reaction mixture was filtered, and the filtrate was washed by saturated sodium thiosulfate solution, dried over sodium sulfate, filtered, and concentrated. The crude residue was purified by silica gel column chromatography using ethyl acetate and dichloromethane as eluents to afford the desired product (1R,2R)-1-((2R,3R,4S,6R)-4-acetoxy-3-(2-acetoxyacetamido)-6-((2,2-dimethyl-4-oxo-3,8,11-trioxa-5-azatridecan-13-yl)oxy)-6-(methoxycarbonyl) tetrahydro-2H-pyran-2-yl)-3-(2-(4-chlorophenyl) acetamido) propane-1,2-diyl diacetate (3) as a colorless sticky liquid. Yield: 500 mg, 42%; LCMS (ESI±) m/z 890.9 [M+H]⁺.

### Synthesis of (1R,2R)-1-((2R,3R,4S,6R)-4-acetoxy-3-(2-acetoxyacetamido)-6-(methoxycarbonyl)-6-(2-(2-(2-((2,2,2-trifluoroacetyl)-14-azaneyl) ethoxy) ethoxy) ethoxy) tetrahydro-2H-pyran-2-yl)-3-(2-(4-chlorophenyl) acetamido) propane-1,2-diyl diacetate (4)

To a stirred solution of (1R,2R)-1-((2R,3R,4S,6R)-4-acetoxy-3-(2-acetoxyacetamido)-6-((2,2-dimethyl-4-oxo-3,8,11-trioxa-5-azatridecan-13-yl) oxy)-6-(methoxycarbonyl) tetrahydro-2H-pyran-2-yl)-3-(2-(4-chlorophenyl) acetamido) propane-1,2-diyl diacetate (3) (500 mg, 0.56 mmol) in anhydrous dichloromethane (2 mL) was added trifluoroacetic acid (1 mL) at 0° C. The reaction mixture was stirred at room temperature for 2 h. After completion, the reaction mixture was concentrated to afford the desired product (1R,2R)-1-((2R,3R,4S,6R)-4-acetoxy-3-(2-acetoxyacetamido)-6-(methoxycarbonyl)-6-(2-(2-(2-((2,2,2-trifluoroacetyl)-14-azaneyl) ethoxy) ethoxy) ethoxy) tetrahydro-2H-pyran-2-yl)-3-(2-(4-chlorophenyl) acetamido) propane-1,2-diyl diacetate (4) as a light yellow sticky syrup. The crude residue was directly used for the next step without further purification. Yield: 500 mg, Crude; LCMS (ESI±) m/z 632.4 [M+H]⁺.

### Synthesis of (1R,2R)-1-((2R,3R,4S,6R)-4-acetoxy-3-(2-acetoxyacetamido)-6-((2,2-dimethyl-4,20-dioxo-3,8,11,14,17,24,27-heptaoxa-5,21-diazanonacosan-29-yl) oxy)-6-(methoxycarbonyl) tetrahydro-2H-pyran-2-yl)-3-(2-(4-chlorophenyl) acetamido) propane-1,2-diyl diacetate (5)

To a stirred solution of (1R,2R)-1-((2R,3R,4S,6R)-4-acetoxy-3-(2-acetoxyacetamido)-6-(methoxycarbonyl)-6-(2-(2-(2-((2,2,2-trifluoroacetyl)-14-azaneyl)ethoxy)ethoxy)ethoxy)tetrahydro-2H-pyran-2-yl)-3-(2-(4-chlorophenyl)acetamido)propane-1,2-diyl diacetate (4) (500 mg, 0.63 mmol) in N, N-Dimethyl formamide (5 mL) were added triethylamine (266.79 mL, 0.02 mmol) and perfluorophenyl 2,2-dimethyl-4-oxo-3,8,11,14,17-pentaoxa-5-azaicosan-20-oate (4) (369 mg, 0.69 mmol) at 0° C. and the reaction mixture was stirred at room temperature for 3 h. After completion the reaction mixture was concentrated, the residue was purified by silica gel column chromatography using methanol and dichloromethane as eluents to afford the desired product (1R,2R)-1-((2R,3R,4S,6R)-4-acetoxy-3-(2-acetoxyacetamido)-6-((2,2-dimethyl-4,20-dioxo-3,8,11,14,17,24,27-heptaoxa-5,21-diazanonacosan-29-yl) oxy)-6-(methoxycarbonyl) tetrahydro-2H-pyran-2-yl)-3-(2-(4-chlorophenyl) acetamido) propane-1,2-diyl diacetate (5) as yellow solid. Yield: 300 mg, 41%; LCMS (ESI) m/z 1137.9 [M+H]⁺.

### Synthesis of (2R,4S,5R,6R)-6-((1R,2R)-3-azido-1,2-dihydroxypropyl)-2-((6-((2-((17,17-dimethyl-15-oxo-3,6,9,12,16-pentaoxaoctadecyl)amino)-3,4-dioxocyclobut-1-en-1-yl)amino)hexyl)oxy)-4-hydroxy-5-(2-hydroxyacetamido) tetrahydro-2H-pyran-2-carboxylic acid (6)

To a stirred solution of (1R,2R)-1-((2R,3R,4S,6R)-4-acetoxy-3-(2-acetoxyacetamido)-6-((2,2-dimethyl-4,20-dioxo-3,8,11,14,17,24,27-heptaoxa-5,21-diazanonacosan-29-yl) oxy)-6-(methoxycarbonyl) tetrahydro-2H-pyran-2-yl)-3-(2-(4-chlorophenyl) acetamido) propane-1,2-diyl diacetate (5) (300 mg, 0.26 mmol) in anhydrous dichloromethane (1 mL) was added trifluoroacetic acid (0.5 mL) at 0° C. The reaction mixture was stirred at room temperature for 2 h. After completion, the reaction mixture was concentrated to afford the desired product (2R,4S,5R,6R)-6-((1R,2R)-3-azido-1,2-dihydroxypropyl)-2-((6-((2-((17,17-dimethyl-15-oxo-3,6,9,12,16-pentaoxaoctadecyl)amino)-3,4-dioxocyclobut-1-en-1-yl)amino)hexyl)oxy)-4-hydroxy-5-(2-hydroxyacetamido) tetrahydro-2H-pyran-2-carboxylic acid (6) as a sticky syrup. The crude residue was directly used for the next step without further purification. Yield: 300 mg Crude.

### Synthesis of (1R,2R)-1-((2R,3R,4S,6R)-4-acetoxy-3-(2-acetoxyacetamido)-6-((2,18-dioxo-6,9,12,15,22,25-hexaoxa-3,19-diazaheptacosan-27-yl) oxy)-6-(methoxycarbonyl) tetrahydro-2H-pyran-2-yl)-3-(2-(4-chlorophenyl) acetamido) propane-1,2-diyl diacetate (7)

To a stirred solution (2R,4S,5R,6R)-6-((1R,2R)-3-azido-1,2-dihydroxypropyl)-2-((6-((2-((17,17-dimethyl-15-oxo-3,6,9,12,16-pentaoxaoctadecyl)amino)-3,4-dioxocyclobut-1-en-1-yl)amino)hexyl)oxy)-4-hydroxy-5-(2-hydroxyacetamido) tetrahydro-2H-pyran-2-carboxylic acid (6) (100 mg, 0.096 mmol) in N, N-Di methyl formamide (1 mL) were added N, N-Dimethyl amino pyridine (12 mg, 0.96 mmol) and acetic anhydride (15 mg, 0.14 mmol) at 0° C. and the reaction mixture was stirred at room temperature for 1 h, After completion the reaction mixture was partitioned between ethyl acetate and water, the organic layer was separated, dried over anhydrous sodium sulfate, and concentrated to afford the desired product (1R,2R)-1-((2R,3R,4S,6R)-4-acetoxy-3-(2-acetoxyacetamido)-6-((2,18-dioxo-6,9,12,15,22,25-hexaoxa-3,19-diazaheptacosan-27-yl)oxy)-6-(methoxycarbonyl)tetrahydro-2H-pyran-2-yl)-3-(2-(4-chlorophenyl)acetamido)propane-1,2-diyl diacetate (7) as white solid which was used as such for the next step. Yield: 100 mg, Crude. LCMS m/z 1077.9 [M−H]⁻.

### Synthesis of (2R,4S,5R,6R)-6-((1R,2R)-3-(2-(4-chlorophenyl) acetamido)-1,2-dihydroxypropyl)-2-((2,18-dioxo-6,9,12,15,22,25-hexaoxa-3,19-diazaheptacosan-27-yl) oxy)-4-hydroxy-5-(2-hydroxyacetamido) tetrahydro-2H-pyran-2-carboxylic acid (237)

To a stirred solution (1R,2R)-1-((2R,3R,4S,6R)-4-acetoxy-3-(2-acetoxyacetamido)-6-((2,18-dioxo-6,9,12,15,22,25-hexaoxa-3,19-diazaheptacosan-27-yl) oxy)-6-(methoxycarbonyl) tetrahydro-2H-pyran-2-yl)-3-(2-(4-chlorophenyl) acetamido) propane-1,2-diyl diacetate (7) (100 mg, 0.96 mmol) in tetrahydrofuran (2 mL) was added a solution of lithium hydroxide monohydrate (100 mg, 0.96 mmol) in water (0.2 mL). The reaction mixture was stirred at room temperature for 6 h. After completion the reaction mixture was treated with acidic resin (Dowex 50, H⁺) to pH˜6 and the suspension was filtered, and the filtrate was concentrated. The crude residue was purified by C18 prep-HPLC using acetonitrile and water (+0.05% TFA) as eluents to afford the desired product (2R,4S,5R,6R)-6-((1R,2R)-3-(2-(4-chlorophenyl) acetamido)-1,2-dihydroxypropyl)-2-((2,18-dioxo-6,9,12,15,22,25-hexaoxa-3,19-diazaheptacosan-27-yl) oxy)-4-hydroxy-5-(2-hydroxyacetamido) tetrahydro-2H-pyran-2-carboxylic acid (237) as white solid. Yield: 20 mg, 42%; LCMS (ESI±) m/z 897 [M+H]⁺.

1H NMR (400 MHz, MeOD): δ 7.33 (t, J=2.5 Hz, 4H), 4.05 (s, 2H), 3.90 (dd, J=22.7, 18.7, 8.9, 5.5 Hz, 5H), 3.75 (t, J=6.1 Hz, 3H), 3.72-3.61 (m, 21H), 3.59-3.54 (m, 6H), 3.42-3.37 (m, 4H), 3.26 (dd, J=13.8, 7.9 Hz, 1H), 2.76 (dd, J=12.8, 4.1 Hz, 1H), 2.49 (t, J=6.2 Hz, 2H), 1.97 (s, 3H), 1.79 (dd, J=12.6, 11.2 Hz, 1H).

### (2R,4S,5R,6R)-6-((1R,2R)-3-(2-(4-chlorophenyl) acetamido)-1,2-dihydroxypropyl)-2-((28-(2,5-dioxo-2,5-dihydro-1H-pyrrol-1-yl)-10,26-dioxo-3,6,13,16,19,22-hexaoxa-9,25-diazaoctacosyl) oxy)-4-hydroxy-5-(2-hydroxyacetamido) tetrahydro-2H-pyran-2-carboxylic acid (Cpd. No. 237M)

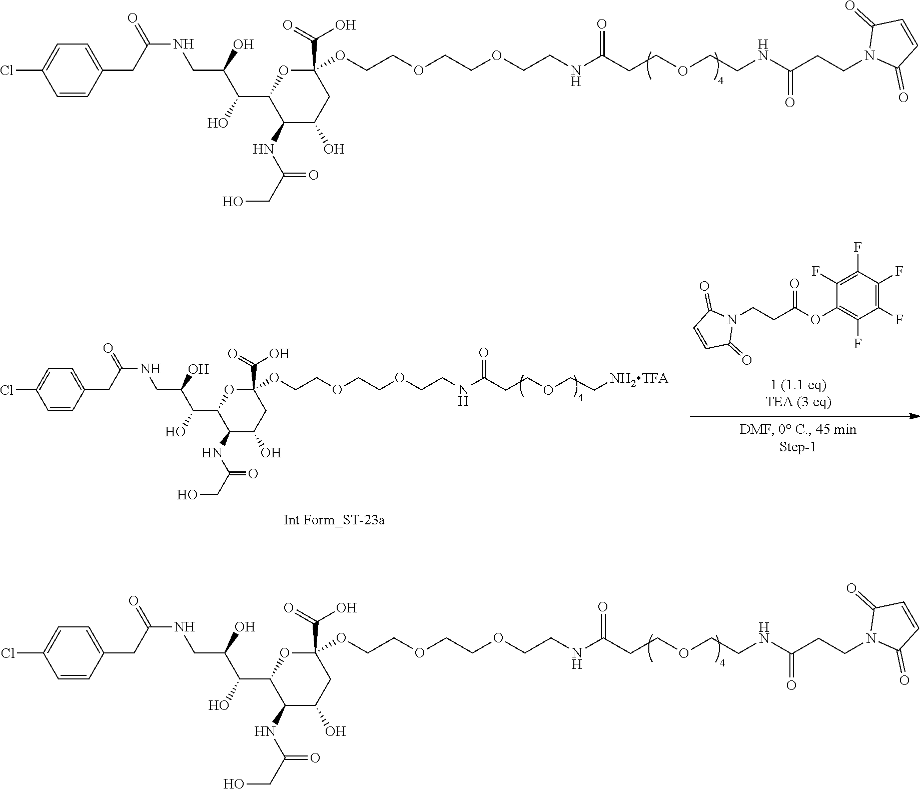

### Synthesis of (2R,4S,5R,6R)-6-((1R,2R)-3-(2-(4-chlorophenyl) acetamido)-1,2-dihydroxypropyl)-2-((28-(2,5-dioxo-2,5-dihydro-1H-pyrrol-1-yl)-10,26-dioxo-3,6,13,16,19,22-hexaoxa-9,25-diazaoctacosyl) oxy)-4-hydroxy-5-(2-hydroxyacetamido) tetrahydro-2H-pyran-2-carboxylic acid (23b)

To a stirred solution of (2R,4S,5R,6R)-6-((1R,2R)-3-(2-(4-chlorophenyl)acetamido)-1,2-dihydroxypropyl)-4-hydroxy-5-(2-hydroxyacetamido)-2-((1,1,1-trifluoro-2,18-dioxo-6,9,12,15,22,25-hexaoxa-314,19-diazaheptacosan-27-yl)oxy)tetrahydro-2H-pyran-2-carboxylic acid (Int form_237) (120 mg, 0.14 mmol) in N, N-Dimethyl formamide (1 mL) were added trimethylamine (0.05 mL, 0.38 mmol) and perfluorophenyl 3-(2,5-dioxo-2,5-dihydro-1H-pyrrol-1-yl) (1) (51 mg, 0.15 mmol) at 0° C. The reaction mixture was stirred at 0° C. for 45 min. After completion, the reaction mixture was concentrated and purified by C18 prep-HPLC using acetonitrile and water (+0.05% TFA) as eluents to afford the desired product (2R,4S,5R,6R)-6-((1R,2R)-3-(2-(4-chlorophenyl) acetamido)-1,2-dihydroxypropyl)-2-((28-(2,5-dioxo-2,5-dihydro-1H-pyrrol-1-yl)-10,26-dioxo-3,6,13,16,19,22-hexaoxa-9,25-diazaoctacosyl) oxy)-4-hydroxy-5-(2-hydroxyacetamido) tetrahydro-2H-pyran-2-carboxylic acid (237M) as white solid. Yield: 26 mg, 20%; LCMS (ESI±) m/z 1006.9 [M+H]⁺.

1H NMR (400 MHz, MeOD): ) δ 7.37-7.28 (m, 4H), 6.85 (s, 2H), 4.05 (s, 2H), 3.97-3.85 (m, 4H), 3.81 (q, J=7.3 Hz, 3H), 3.77-3.70 (m, 4H), 3.66 (d, J=4.2 Hz, 9H), 3.63 (ddd, J=7.3, 3.5, 1.7 Hz, 14H), 3.60-3.54 (m, 5H), 3.52 (d, J=5.5 Hz, 2H), 3.42-3.38 (m, 3H), 3.25 (dd, J=13.8, 8.0 Hz, 1H), 2.77 (dd, J=12.7, 4.2 Hz, 1H), 2.49 (td, J=6.5, 1.9 Hz, 4H), 1.77 (t, J=11.9 Hz, 1H).

### (2R,4S,5R,6R)-6-((1R,2R)-3-(2-([1,1′-biphenyl]-4-yl) acetamido)-1,2-dihydroxypropyl)-2-((2,19-dioxo-6,9,12,15,22-pentaoxa-3,18-diazatetracosan-24-yl) oxy)-4-hydroxy-5-(2-hydroxyacetamido) tetrahydro-2H-pyran-2-carboxylic acid (Cpd. 238)

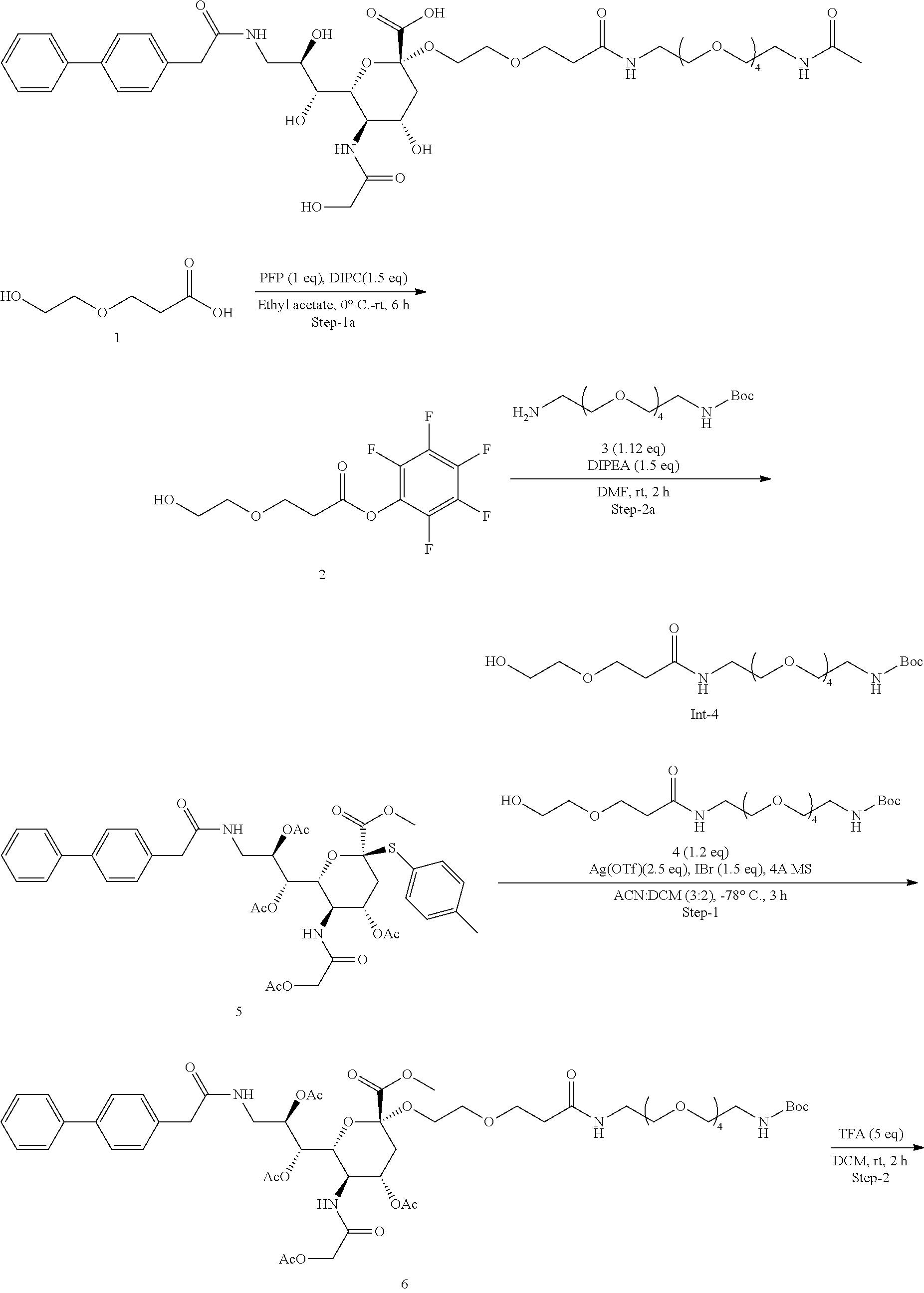

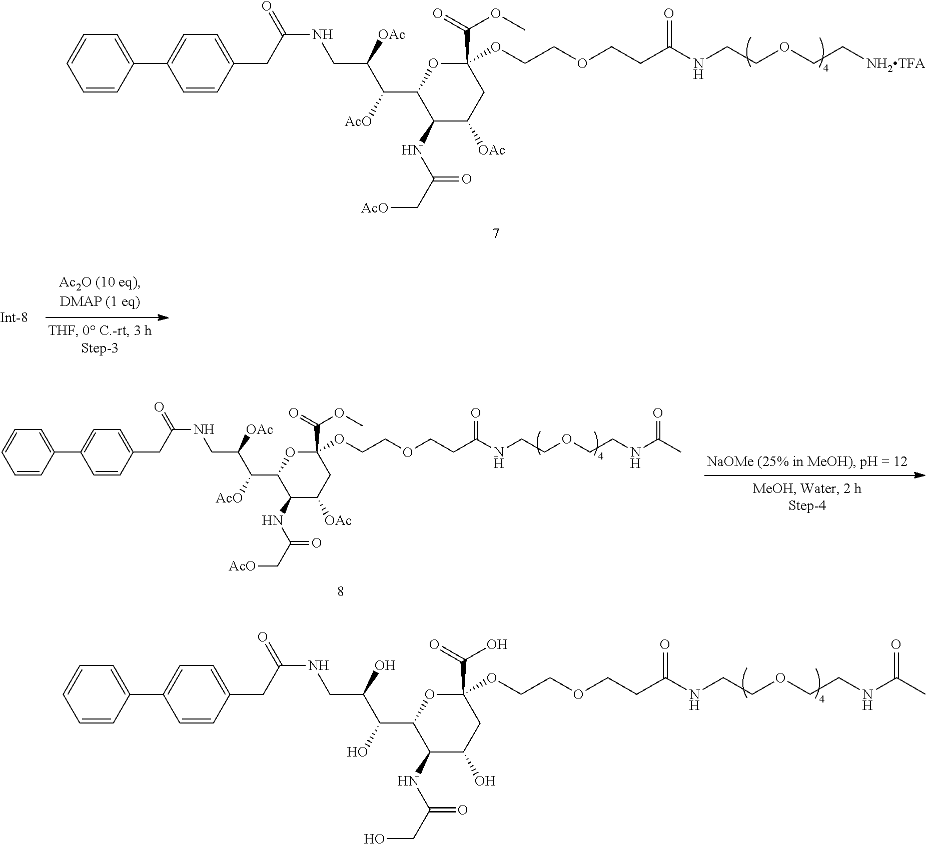

### Synthesis of perfluorophenyl 3-(2-hydroxyethoxy) propanoate (2)

To a stirred solution of 3-(2-hydroxyethoxy) propanoic acid (1) (1 g, 7.46 mmol) and penta-fluoro phenol (1.37 g, 7.46 mmol) in dry ethyl acetate (50 mL) was added diisopropyl carbodiimide (1.76 ml, 11.19 mmol) dropwise at 0° C. The reaction mixture was stirred at room temperature for next 6 h. After completion, the reaction mixture was filtered, and the filtrate was concentrated. The crude residue was purified by silica gel column chromatography using ethyl acetate and Hexane as eluents to afford perfluorophenyl 3-(2-hydroxyethoxy) propanoate (2) as off white semi-solid compound. Yield:1.9 g, 85.20%. ¹H NMR (400 MHz, MeOD) δ 5.50 (t, J=2 Hz, 1H), 3.91 (t, J=0.8 Hz, 2H), 3.68 (t, J=1.2 Hz, 2H), 3.59 (t, J=1.2 Hz, 2H), 3.04 (t, J=0.8 Hz, 2H).

### Synthesis of tert-butyl (21-hydroxy-16-oxo-3,6,9,12,19-pentaoxa-15-azahenicosyl) carbamate (Int-4)

To a stirred solution of perfluorophenyl 3-(2-hydroxyethoxy)propanoate (2) (1.9 g, 6.33 mmol) in N, N-Dimethyl formamide (20 mL) were added tert-butyl (14-amino-3,6,9,12-tetraoxatetradecyl)carbamate (3) (2.38 g, 7.09 mmol) and di-isopropyl ethyl amine (1.75 ml, 9.5 mmol) at 0° C. The reaction mixture was stirred at room temperature for 2 h. After completion, the reaction mixture was purified chromatography using acetonitrile and water as eluents to afford tert-butyl tert-butyl (21-hydroxy-16-oxo-3,6,9,12,19-pentaoxa-15-azahenicosyl) carbamate (Int-4) as white solid. Yield:2.4 g, 83.91%; LCMS (ESI±) m/z 453.4 [M+H]⁺.

### Synthesis of (1R,2R)-3-(2-([1,1′-biphenyl]-4-yl) acetamido)-1-((2R,3R,4S,6R)-4-acetoxy-3-(2-acetoxyacetamido)-6-((2,2-dimethyl-4,21-dioxo-3,8,11,14,17,24-hexaoxa-5,20-diazahexacosan-26-yl) oxy)-6-(methoxycarbonyl) tetrahydro-2H-pyran-2-yl) propane-1,2-diyl diacetate (6)

To a stirred solution of (1R,2R)-3-(2-([1,1′-biphenyl]-4-yl)acetamido)-1-((2R,3R,4S,6R)-4-acetoxy-3-(2-acetoxyacetamido)-6-(methoxycarbonyl)-6-(p-tolylthio)tetrahydro-2H-pyran-2-yl)propane-1,2-diyl diacetate (5) (1.2 g, 1.48 mmol) and tert-butyl (24-hydroxy-15-oxo-3,6,9,12,19,22-hexaoxa-16-azatetracosyl)carbamate (4) (1.1 g, 2.23 mmol, 1.5 eq.) in anhydrous acetonitrile (12 mL) and anhydrous dichloromethane (8 mL) were added powdered molecular sieves 4 Å (4 g), Ag(OTf) (0.57 g, 2.23 mmol) and the reaction mixture was stirred under nitrogen atmosphere at room temperature for 1 h. The mixture was cooled to −78° C. and a solution of iodine bromide (0.45 g, 2.23 mmol) in DCM (4 ml) was added. The reaction mixture was stirred at the same temperature for 2 h. After completion, the reaction mixture was diluted with dichloromethane (15 ml), quenched with triethylamine (up to pH=7), filtered, the filtrate was washed with 10% sodium thio-sulfate solution, dried over sodium sulfate, then concentrated. The crude residue was purified by C18 reverse phase column chromatography using acetonitrile and water (+0.05% TFA) as eluents to afford the desired product (1R,2R)-3-(2-([1,1′-biphenyl]-4-yl) acetamido)-1-((2R,3R,4S,6R)-4-acetoxy-3-(2-acetoxyacetamido)-6-((2,2-dimethyl-4,21-dioxo-3,8,11,14,17,24-hexaoxa-5,20-diazahexacosan-26-yl) oxy)-6-(methoxycarbonyl) tetrahydro-2H-pyran-2-yl) propane-1,2-diyl diacetate (6) as yellow solid. Yield: 0.9 g, 53.57%; LCMS (ESI±) m/z 1136.9 [M+H]⁺.

### Synthesis of (1R,2R)-3-(2-([1,1′-biphenyl]-4-yl) acetamido)-1-((2R,3R,4S,6R)-4-acetoxy-3-(2-acetoxyacetamido)-6-(methoxycarbonyl)-6-((1,1,1-trifluoro-2,19-dioxo-6,9,12,15,22-pentaoxa-314,18-diazatetracosan-24-yl) oxy) tetrahydro-2H-pyran-2-yl) propane-1,2-diyl diacetate (7)

To a stirred solution of (1R,2R)-3-(2-([1,1′-biphenyl]-4-yl) acetamido)-1-((2R,3R,4S,6R)-4-acetoxy-3-(2-acetoxyacetamido)-6-((2,2-dimethyl-4,21-dioxo-3,8,11,14,17,24-hexaoxa-5,20-diazahexacosan-26-yl) oxy)-6-(methoxycarbonyl) tetrahydro-2H-pyran-2-yl) propane-1,2-diyl diacetate (6) (0.1 g, 0.08 mmol) in anhydrous dichloromethane (1 mL), trifluoro acetic acid (0.06 mL, 0.44 mmol) was added at 0° C. The reaction mixture was stirred at room temperature for 2 h. After completion, the reaction mixture was concentrated. The crude residue was suspended in methanol and neutralized with tetra alkyl ammonium carbonate, polymer-bound resin. The reaction mixture was filtered, and the filtrate was concentrate to afford the desired product (1R,2R)-3-(2-([1,1′-biphenyl]-4-yl) acetamido)-1-((2R,3R,4S,6R)-4-acetoxy-3-(2-acetoxyacetamido)-6-(methoxycarbonyl)-6-((1,1,1-trifluoro-2,19-dioxo-6,9,12,15,22-pentaoxa-314,18-diazatetracosan-24-yl) oxy) tetrahydro-2H-pyran-2-yl) propane-1,2-diyl diacetate (7) as yellow solid. Yield: 90 mg; crude. LCMS (ESI±) m/z 1035.9 [M+H]⁺.

### Synthesis of (1R,2R)-3-(2-([1,1′-biphenyl]-4-yl) acetamido)-1-((2R,3R,4S,6R)-4-acetoxy-3-(2-acetoxyacetamido)-6-((2,19-dioxo-6,9,12,15,22-pentaoxa-3,18-diazatetracosan-24-yl) oxy)-6-(methoxycarbonyl) tetrahydro-2H-pyran-2-yl) propane-1,2-diyl diacetate (8)

To a stirred solution of (1R,2R)-3-(2-([1,1′-biphenyl]-4-yl)acetamido)-1-((2R,3R,4S,6R)-4-acetoxy-3-(2-acetoxyacetamido)-6-(methoxycarbonyl)-6-((1,1,1-trifluoro-2,19-dioxo-6,9,12,15,22-pentaoxa-314,18-diazatetracosan-24-yl)oxy)tetrahydro-2H-pyran-2-yl)propane-1,2-diyl diacetate (7) (0.19 g, 0.18 mmol) in anhydrous tetrahydrofuran (2 mL), N,N-Dimethyl amino pyridine (22 mg, 0.18 mmol) was added. After 10 min of stirring at rt, the mixture was cooled to 0° C., then acetic anhydride (0.01 mL, 1.83 mmol) was added, and the reaction mixture was stirred for 3 h. After completion, the reaction mixture was concentrated, the crude residue was purified by C18 reverse phase column chromatography using acetonitrile and water (+0.05% TFA) as eluents to afford the desired product ((1R,2R)-3-(2-([1,1′-biphenyl]-4-yl) acetamido)-1-((2R,3R,4S,6R)-4-acetoxy-3-(2-acetoxyacetamido)-6-((2,19-dioxo-6,9,12,15,22-pentaoxa-3,18-diazatetracosan-24-yl) oxy)-6-(methoxycarbonyl) tetrahydro-2H-pyran-2-yl) propane-1,2-diyl diacetate (8) as yellow solid. Yield:145 mg, 73.60%; LCMS (ESI) m/z 1078.1 [M+H]⁺.

### Synthesis of (2R,4S,5R,6R)-6-((1R,2R)-3-(2-([1,1′-biphenyl]-4-yl) acetamido)-1,2-dihydroxypropyl)-2-((2,19-dioxo-6,9,12,15,22-pentaoxa-3,18-diazatetracosan-24-yl) oxy)-4-hydroxy-5-(2-hydroxyacetamido) tetrahydro-2H-pyran-2-carboxylic acid (238)

To a stirred solution of (1R,2R)-3-(2-([1,1′-biphenyl]-4-yl) acetamido)-1-((2R,3R,4S,6R)-4-acetoxy-3-(2-acetoxyacetamido)-6-((2,19-dioxo-6,9,12,15,22-pentaoxa-3,18-diazatetracosan-24-yl) oxy)-6-(methoxycarbonyl) tetrahydro-2H-pyran-2-yl) propane-1,2-diyl diacetate (9) (145 mg, 0.13 mmol) in methanol (1.8 mL) and water (0.2 mL), was dropwise added sodium methoxide (25% in methanol) (up to pH˜12) at 0° C. The reaction mixture was stirred at room temperature for 6 h. After completion, the reaction mixture was concentrated and purified by C18 prep-HPLC using acetonitrile and water (+0.05% TFA) as eluents to afford the desired product (2R,4S,5R,6R)-6-((1R,2R)-3-(2-([1,1′-biphenyl]-4-yl) acetamido)-1,2-dihydroxypropyl)-2-((2,19-dioxo-6,9,12,15,22-pentaoxa-3,18-diazatetracosan-24-yl) oxy)-4-hydroxy-5-(2-hydroxyacetamido) tetrahydro-2H-pyran-2-carboxylic acid (238) as white solid. Yield: 24 mg, 20%; LCMS (ESI±) m/z 895.2 [M+H]⁺. ¹H NMR (400 MHz, MeOD) δ 7.62 (dd, J=11.3, 8.1 Hz, 4H), 7.46-7.34 (m, 5H), 4.05 (s, 2H), 3.96-3.85 (m, 4H), 3.85-3.48 (m, 26H), 3.40 (q, J=7.1 Hz, 4H), 3.35-3.23 (m, 1H), 2.74 (dd, J=12.8 Hz, 1H), 2.53-2.43 (m, 2H), 1.96 (s, 3H), 1.78 (t, J=11.8 Hz, 1H).

### (2R,4S,5R,6R)-6-((1R,2R)-3-(2-([1,1′-biphenyl]-4-yl) acetamido)-1,2-dihydroxypropyl)-2-((25-(2,5-dioxo-2,5-dihydro-1H-pyrrol-1-yl)-6,23-dioxo-3,10,13,16,19-pentaoxa-7,22-diazapentacosyl) oxy)-4-hydroxy-5-(2-hydroxyacetamido) tetrahydro-2H-pyran-2-carboxylic Acid (CPD. 238M)

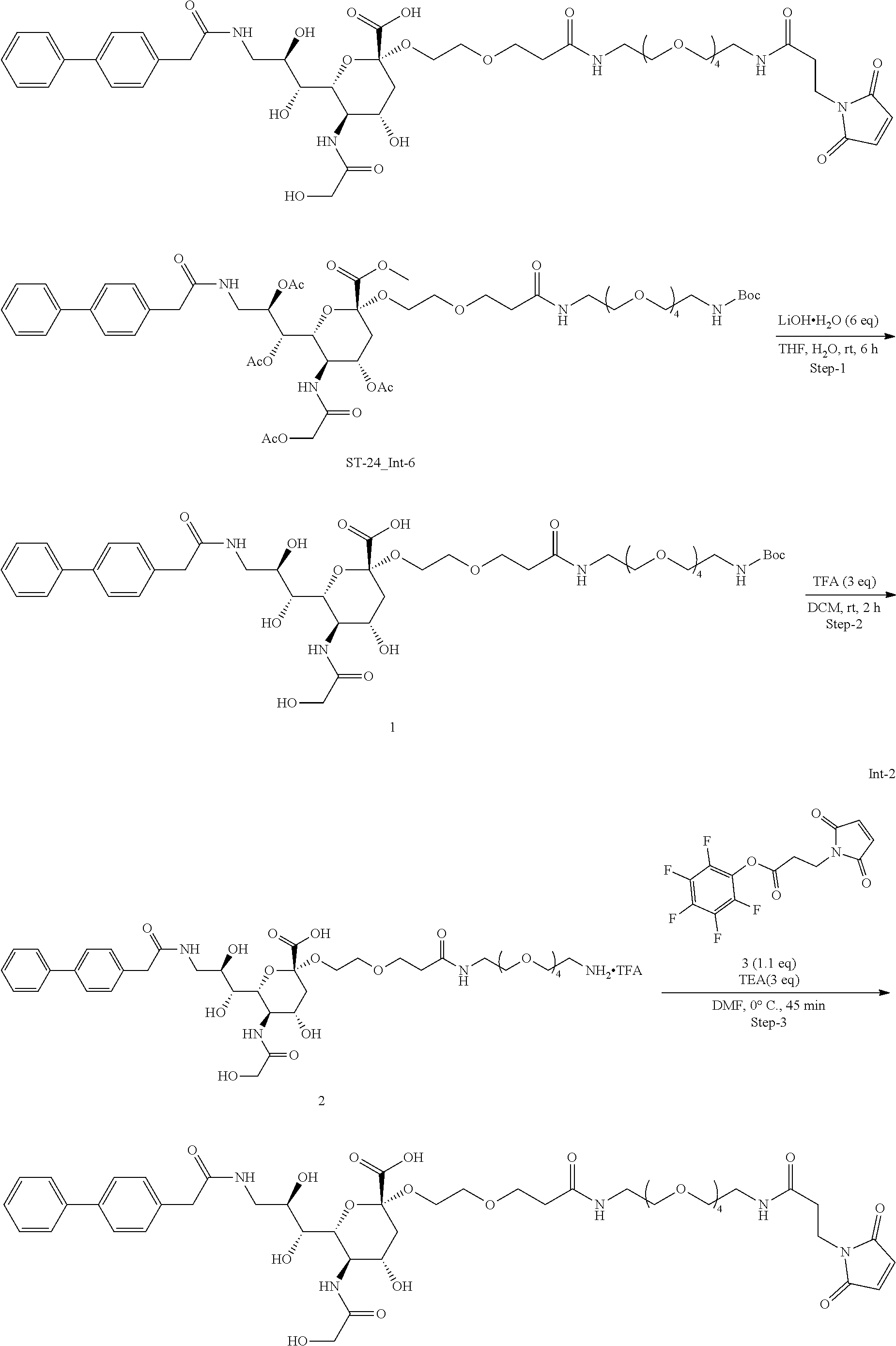

### Synthesis of (2R,4S,5R,6R)-6-((1R,2R)-3-(2-([1,1′-biphenyl]-4-yl) acetamido)-1,2-dihydroxypropyl)-2-((2,2-dimethyl-4,21-dioxo-3,8,11,14,17,24-hexaoxa-5,20-diazahexacosan-26-yl) oxy)-4-hydroxy-5-(2-hydroxyacetamido) tetrahydro-2H-pyran-2-carboxylic acid (1)

To a stirred solution of (1R,2R)-3-(2-([1,1′-biphenyl]-4-yl) acetamido)-1-((2R,3R,4S,6R)-4-acetoxy-3-(2-acetoxyacetamido)-6-((2,2-dimethyl-4,21-dioxo-3,8,11,14,17,24-hexaoxa-5,20-diazahexacosan-26-yl) oxy)-6-(methoxycarbonyl) tetrahydro-2H-pyran-2-yl) propane-1,2-diyl diacetate (ST-24_Int-6) (0.26 g, 0.22 mmol) in tetrahydrofuran (2.7 mL) was added a solution of lithium hydroxide monohydrate (57 mg, 1.37 mmol) in water (0.3 mL), at 0° C. and the reaction mixture was stirred at room temperature for 6 h. After completion the reaction mixture was treated with acidic resin (Dowex 50, H⁺) to pH˜6, the suspension was filtered, and the filtrate was concentrated. The crude residue was purified by C18 reverse phase column chromatography using acetonitrile and water (+0.05% TFA) as eluents to afford the desired product (2R,4S,5R,6R)-6-((1R,2R)-3-(2-([1,1′-biphenyl]-4-yl) acetamido)-1,2-dihydroxypropyl)-2-((2,2-dimethyl-4,21-dioxo-3,8,11,14,17,24-hexaoxa-5,20-diazahexacosan-26-yl) oxy)-4-hydroxy-5-(2-hydroxyacetamido) tetrahydro-2H-pyran-2-carboxylic acid (1) as off white solid. Yield: 0.2 g, 91%; LCMS (ESI±) m/z 953.8 [M+H]⁺.

### Synthesis of (2R,4S,5R,6R)-6-((1R,2R)-3-(2-([1,1′-biphenyl]-4-yl) acetamido)-1,2-dihydroxypropyl)-4-hydroxy-5-(2-hydroxyacetamido)-2-((1,1,1-trifluoro-2,19-dioxo-6,9,12,15,22-pentaoxa-314,18-diazatetracosan-24-yl) oxy) tetrahydro-2H-pyran-2-carboxylic acid (2)

To a stirred solution of (2R,4S,5R,6R)-6-((1R,2R)-3-(2-([1,1′-biphenyl]-4-yl) acetamido)-1,2-dihydroxypropyl)-2-((2,2-dimethyl-4,21-dioxo-3,8,11,14,17,24-hexaoxa-5,20-diazahexacosan-26-yl) oxy)-4-hydroxy-5-(2-hydroxyacetamido) tetrahydro-2H-pyran-2-carboxylic acid (1) (0.19 g, 0.19 mmol) in anhydrous dichloromethane (2 mL), was added trifluoro acetic acid (0.5 mL) at 0° C. The reaction mixture was stirred at room temperature for 2 h. After completion, the solvent was concentrated, and the residue obtained was suspended in methanol and neutralized with tetra alkyl ammonium carbonate (polymer-bound resin). The suspension was filtered and the filtrate was concentrated to afford the desired product (2R,4S,5R,6R)-6-((1R,2R)-3-(2-([1,1′-biphenyl]-4-yl) acetamido)-1,2-dihydroxypropyl)-4-hydroxy-5-(2-hydroxyacetamido)-2-((1,1,1-trifluoro-2,19-dioxo-6,9,12,15,22-pentaoxa-314,18-diazatetracosan-24-yl) oxy) tetrahydro-2H-pyran-2-carboxylic acid (2) as yellow solid. Yield:120 mg, 71%; LCMS (ESI±) m/z 853.9, [M+H]⁺.

### Synthesis of (2R,4S,5R,6R)-6-((1R,2R)-3-(2-(4-chlorophenyl) acetamido)-1,2-dihydroxypropyl)-2-((1-(2,5-dioxo-2,5-dihydro-1H-pyrrol-1-yl)-3,19-dioxo-7,10,13,16-tetraoxa-4,20-diazapentacosan-25-yl) oxy)-4-hydroxy-5-(2-hydroxyacetamido) tetrahydro-2H-pyran-2-carboxylic acid (238M)

To a stirred solution (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-((1,1,1-trifluoro-2,19-dioxo-6,9,12,15,22-pentaoxa-314,18-diazatetracosan-24-yl)oxy)tetrahydro-2H-pyran-2-carboxylic acid (2) (120 mg, 0.11 mmol) in N, N-dimethyl formamide (1.2 mL) were added 2,5-dioxopyrrolidin-1-yl 3-(2,5-dioxo-2,5-dihydro-1H-pyrrol-1-yl)propanoate (3) (49.3 mg, 0.18 mmol) and triethyl amine (0.55 ml, 0.37 mmol) at 0° C. The reaction mixture was stirred at room temperature for 1 h. After completion, the reaction mixture was purified by C18 prep-HPLC using acetonitrile and water (+0.05% TFA) as eluents to afford the desired product (2R,4S,5R,6R)-6-((1R,2R)-3-(2-(4-chlorophenyl) acetamido)-1,2-dihydroxypropyl)-2-((1-(2,5-dioxo-2,5-dihydro-1H-pyrrol-1-yl)-3,19-dioxo-7,10,13,16-tetraoxa-4,20-diazapentacosan-25-yl) oxy)-4-hydroxy-5-(2-hydroxyacetamido) tetrahydro-2H-pyran-2-carboxylic acid (238M) as white solid. Yield: 34 mg, 27%; LCMS (ESI±) m/z 1005 [M+H]⁺. ¹H NMR (400 MHz, MeOD) δ 7.62 (dd, J=11.3, 8.1 Hz, 4H), 7.49-7.31 (m, 5H), 6.84 (d, J=1.1 Hz, 2H), 4.05 (s, 2H), 3.96-3.85 (m, 4H), 3.85-3.48 (m, 27H), 3.40 (q, J=7.1 Hz, 4H), 3.35-3.23 (m, 1H), 2.70 (dd, J=12.8 Hz, 1H), 2.53-2.43 (m, 4H), 1.78 (t, J=11.8 Hz, 1H).

### (2R,4S,5R,6R)-6-((1R,2R)-3-(2-(4-chlorophenyl) acetamido)-1,2-dihydroxypropyl)-2-((2,19-dioxo-6,9,12,15,22-pentaoxa-3,18-diazatetracosan-24-yl) oxy)-4-hydroxy-5-(2-hydroxyacetamido) tetrahydro-2H-pyran-2-carboxylic acid (Cpd. 239)

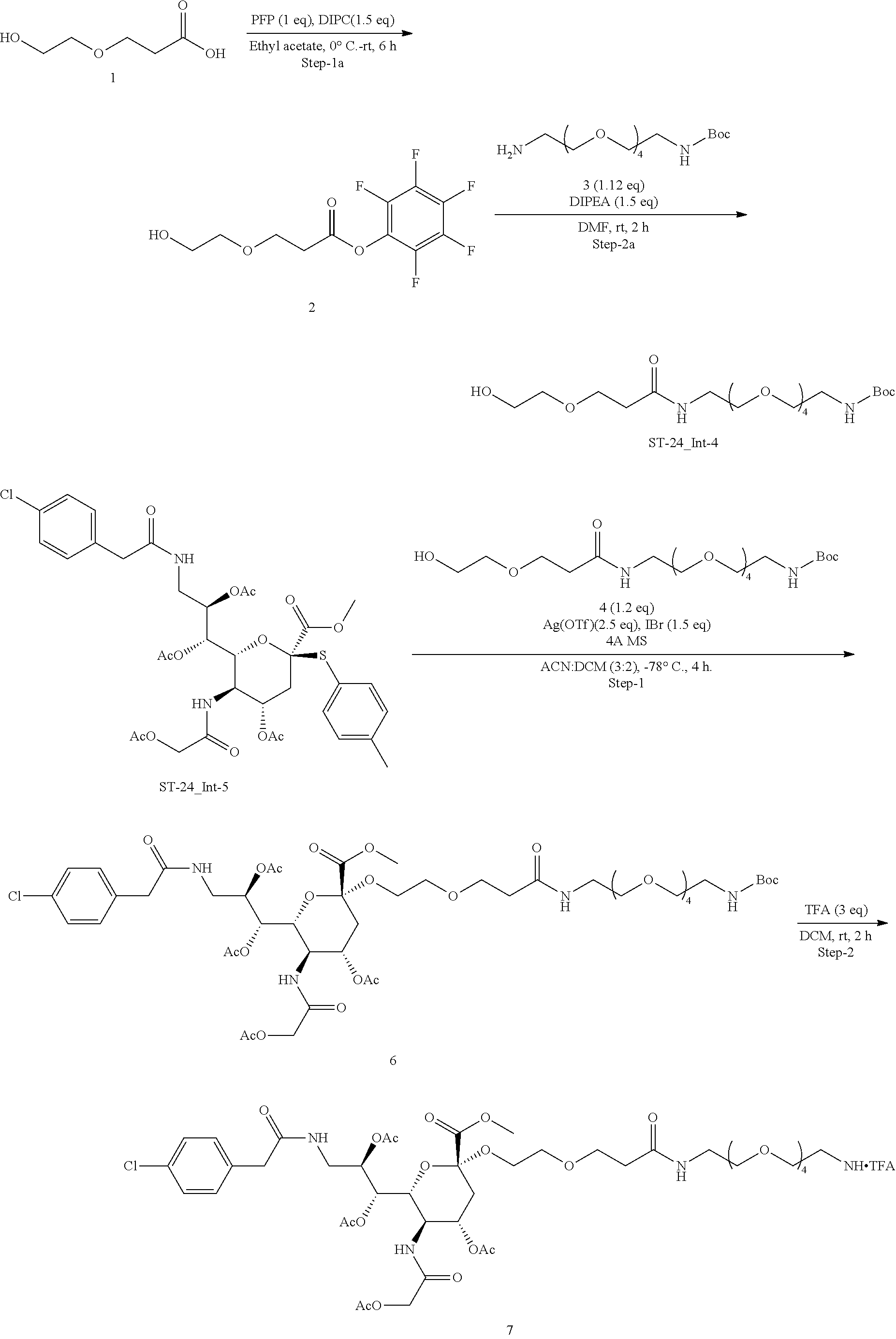

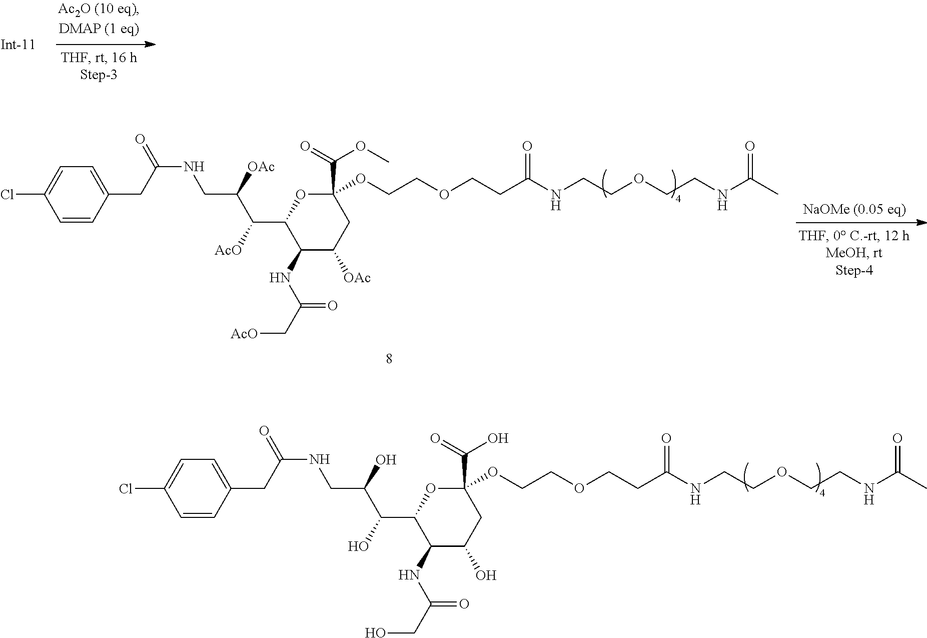

### Synthesis of perfluorophenyl 3-(2-hydroxyethoxy)propanoate (2)

To a stirred solution of 3-(2-hydroxyethoxy) propanoic acid (1) (1 g, 7.46 mmol) and penta-fluoro phenol (1.37 g, 7.46 mmol) in dry ethyl acetate (50 mL) was dropwise added diisopropyl carbodiimide (1.76 ml, 11.19 mmol) at 0° C. and the reaction mixture was stirred at room temperature for 6 h. After completion, the reaction mixture was filtered, and the filtrate was concentrated. The crude residue was purified by silica gel column chromatography using ethyl acetate and hexane as eluents to afford perfluorophenyl 3-(2-hydroxyethoxy) propanoate (2) as off white semi solid. Yield: 1.9 g, 85%. ¹H NMR (400 MHz, MeOD) δ 5.50 (t, J=2 Hz, 1H), 3.91 (t, J=0.8 Hz, 2H), 3.68 (t, J=1.2 Hz, 2H), 3.59 (t, J=1.2 Hz, 2H), 3.04 (t, J=0.8 Hz, 2H).

### Synthesis of tert-butyl (21-hydroxy-16-oxo-3,6,9,12,19-pentaoxa-15-azahenicosyl) carbamate (ST-24_Int-4)

To a stirred solution of perfluorophenyl 3-(2-hydroxyethoxy)propanoate (2,1.9 g, 6.33 mmol) in N,N-Dimethyl formamide (20 mL) were added tert-butyl (14-amino-3,6,9,12-tetraoxatetradecyl)carbamate (3) (2.38 g, 7.09 mmol) and diisopropyl ethylamine (1.75 ml, 9.5 mmol) at 0° C. The reaction mixture was stirred at room temperature for 2 h. After completion, the reaction mixture was purified by C18 prep-HPLC using acetonitrile and water (0.05%, TFA) to afford tert-butyl (21-hydroxy-16-oxo-3,6,9,12,19-pentaoxa-15-azahenicosyl) carbamate (ST-24_Int-4) as white solid. Yield: 2.4 g, 84%; LCMS (ESI±) m/z 453.4 [M+H]⁺.

### Synthesis of (1R,2R)-1-((2R,3R,4S,6R)-4-acetoxy-3-(2-acetoxyacetamido)-6-((2,2-dimethyl-4,21-dioxo-3,8,11,14,17,24-hexaoxa-5,20-diazahexacosan-26-yl) oxy)-6-(methoxycarbonyl) tetrahydro-2H-pyran-2-yl)-3-(2-(4-chlorophenyl) acetamido) propane-1,2-diyl diacetate (6)

To a stirred solution of (1R,2R)-1-((2R,3R,4S,6R)-4-acetoxy-3-(2-acetoxyacetamido)-6-(methoxycarbonyl)-6-(p-tolylthio) tetrahydro-2H-pyran-2-yl)-3-(2-(4-chlorophenyl) acetamido) propane-1,2-diyl diacetate (ST-24_Int-5) (1.0 g, 1.30 mmol) and tert-butyl (21-hydroxy-16-oxo-3,6,9,12,19-pentaoxa-15-azahenicosyl) carbamate (4) (0.88 g, 1.96 mmol) in anhydrous dichloromethane (12 mL) and anhydrous acetonitrile (24 mL) were added Silver (+1) trifluoro methane sulfonate (Ag(OTf) (0.83 g, 3.27 mmol) and activated 4 Å powdered molecular sieves (5 g) and the reaction mixture was stirred at room temperature for 1 h under nitrogen atmosphere. The reaction mixture was cooled to −78° C. and a solution of iodine monobromide (0.40 g, 1.96 mmol.) dissolved in dichloromethane (4 ml) was added dropwise. The reaction mixture was stirred at the same temperature for 2 h. After completion, the reaction mixture was quenched by addition of triethylamine (5 mL), filtered, and the filtrate was washed with a saturated solution of sodium thiosulfate, dried over sodium sulfate, filtered and concentrated. The crude residue was purified by C18 reverse phase column chromatography using acetonitrile and water (+0.05%, TFA) as eluents to afford the desired product (1R,2R)-1-((2R,3R,4S,6R)-4-acetoxy-3-(2-acetoxyacetamido)-6-((2,2-dimethyl-4,21-dioxo-3,8,11,14,17,24-hexaoxa-5,20-diazahexacosan-26-yl) oxy)-6-(methoxycarbonyl) tetrahydro-2H-pyran-2-yl)-3-(2-(4-chlorophenyl) acetamido) propane-1,2-diyl diacetate (6) as yellow solid. Yield: 0.5 g, 34.98%; LCMS (ESI±) m/z 1093.9 [M+H]⁺.

### Synthesis of (1R,2R)-1-((2R,3R,4S,6R)-4-acetoxy-3-(2-acetoxyacetamido)-6-(methoxycarbonyl)-6-((1,1,1-trifluoro-2,19-dioxo-6,9,12,15,22-pentaoxa-314,18-diazatetracosa-24-yl) oxy) tetrahydro-2H-pyran-2-yl)-3-(2-(4-chlorophenyl) acetamido) propane-1,2-diyl diacetate (7)

To a stirred solution of (1R,2R)-1-((2R,3R,4S,6R)-4-acetoxy-3-(2-acetoxyacetamido)-6-((2,2-dimethyl-4,21-dioxo-3,8,11,14,17,24-hexaoxa-5,20-diazahexacosan-26-yl) oxy)-6-(methoxycarbonyl) tetrahydro-2H-pyran-2-yl)-3-(2-(4-chlorophenyl) acetamido) propane-1,2-diyl diacetate (6) (0.2 g, 0.18 mmol) in anhydrous dichloromethane (2 mL) and trifluoro acetic acid (0.007 mL, 0.91 mmol) was added at 0° C. The reaction mixture was stirred at room temperature for 2 h. After completion, the reaction mixture was concentrated, suspended in methanol, neutralized with tetra alkyl ammonium carbonate, polymer-bound resin. After neutralization the reaction mixture was filtered, and the filtrate was concentrated to afford (1R,2R)-1-((2R,3R,4S,6R)-4-acetoxy-3-(2-acetoxyacetamido)-6-(methoxycarbonyl)-6-((1,1,1-trifluoro-2,19-dioxo-6,9,12,15,22-pentaoxa-314,18-diazatetracosan-24-yl) oxy) tetrahydro-2H-pyran-2-yl)-3-(2-(4-chlorophenyl) acetamido) propane-1,2-diyl diacetate (7) as yellow solid. Yield: 200 mg (Crude); LCMS (ESI±) m/z 993.8 [M+H]⁺.

### Synthesis of (1R,2R)-1-((2R,3R,4S,6R)-4-acetoxy-3-(2-acetoxyacetamido)-6-((2,19-dioxo-6,9,12,15,22-pentaoxa-3,18-diazatetracosan-24-yl) oxy)-6-(methoxycarbonyl) tetrahydro-2H-pyran-2-yl)-3-(2-(4-chlorophenyl) acetamido) propane-1,2-diyl diacetate (8)

To a stirred solution of (1R,2R)-1-((2R,3R,4S,6R)-4-acetoxy-3-(2-acetoxyacetamido)-6-(methoxycarbonyl)-6-((1,1,1-trifluoro-2,19-dioxo-6,9,12,15,22-pentaoxa-314,18-diazatetracosan-24-yl)oxy)tetrahydro-2H-pyran-2-yl)-3-(2-(4-chlorophenyl)acetamido)propane-1,2-diyl diacetate (7) (0.2 g, 0.18 mmol) in anhydrous tetrahydrofuran (4 mL), were added N,N-Dimethyl amino pyridine (22 mg, 0.18 mmol), acetic anhydride (0.17 mL, 1.82 mmol) at 0° C. and the reaction mixture was stirred for 3 h at room temperature. After completion, the reaction mixture was concentrated and purified by C18 reverse phase column chromatography using acetonitrile and water (0.05%, TFA) as eluents to afford the desired product (1R,2R)-1-((2R,3R,4S,6R)-4-acetoxy-3-(2-acetoxyacetamido)-6-((2,19-dioxo-6,9,12,15,22-pentaoxa-3,18-diazatetracosan-24-yl) oxy)-6-(methoxycarbonyl) tetrahydro-2H-pyran-2-yl)-3-(2-(4-chlorophenyl) acetamido) propane-1,2-diyl diacetate (8) as yellow solid. Yield:120 mg, 63%; LCMS (ESI±) m/z 1033.9 [M−H]⁻.

### Synthesis of (2R,4S,5R,6R)-6-((1R,2R)-3-(2-(4-chlorophenyl) acetamido)-1,2-dihydroxypropyl)-2-((2,19-dioxo-6,9,12,15,22-pentaoxa-3,18-diazatetracosan-24-yl) oxy)-4-hydroxy-5-(2-hydroxyacetamido) tetrahydro-2H-pyran-2-carboxylic acid (239)

To a stirred solution of (1R,2R)-1-((2R,3R,4S,6R)-4-acetoxy-3-(2-acetoxyacetamido)-6-((2,19-dioxo-6,9,12,15,22-pentaoxa-3,18-diazatetracosan-24-yl) oxy)-6-(methoxycarbonyl) tetrahydro-2H-pyran-2-yl)-3-(2-(4-chlorophenyl) acetamido) propane-1,2-diyl diacetate (8) (120 mg, 0.11 mmol) in methanol (2.7 mL) and water (0.3 mL), was added sodium methoxide in (25% in methanol) (0.01 ml, 0.05 mmol) dropwise at 0° C. The reaction mixture was stirred at room temperature for 12 h. After completion, the reaction mixture was concentrated and the residue obtained was purified by C18 prep-HPLC using acetonitrile and water (+0.05%, TFA) as eluents to afford the desired product (2R,4S,5R,6R)-6-((1R,2R)-3-(2-(4-chlorophenyl) acetamido)-1,2-dihydroxypropyl)-2-((2,19-dioxo-6,9,12,15,22-pentaoxa-3,18-diazatetracosan-24-yl) oxy)-4-hydroxy-5-(2-hydroxyacetamido) tetrahydro-2H-pyran-2-carboxylic acid (239) as white solid. Yield: 40 mg, 40%; LCMS (ESI±) m/z 853.31 [M+H]⁺. ¹H NMR (400 MHz, MeOH-d₄) δ 7.28 (s, 4H), 4.10 (q, J=2.3 Hz, 2H), 3.85-3.71 (m, 5H), 3.72-3.52 (m, 22H), 3.37-3.19 (m, 8H), 2.70 (dd, J=12.8 Hz, 1H), 2.43 (d, J=6 Hz, 2H), 1.93 (s, 3H), 1.75 (t, J=12 Hz, 1H).

### (2R,4S,5R,6R)-6-((1R,2R)-3-(2-(4-chlorophenyl) acetamido)-1,2-dihydroxypropyl)-2-((25-(2,5-dioxo-2,5-dihydro-1H-pyrrol-1-yl)-6,23-dioxo-3,10,13,16,19-pentaoxa-7,22-diazapentacosyl) oxy)-4-hydroxy-5-(2-hydroxyacetamido) tetrahydro-2H-pyran-2-carboxylic acid (Compound 239M)

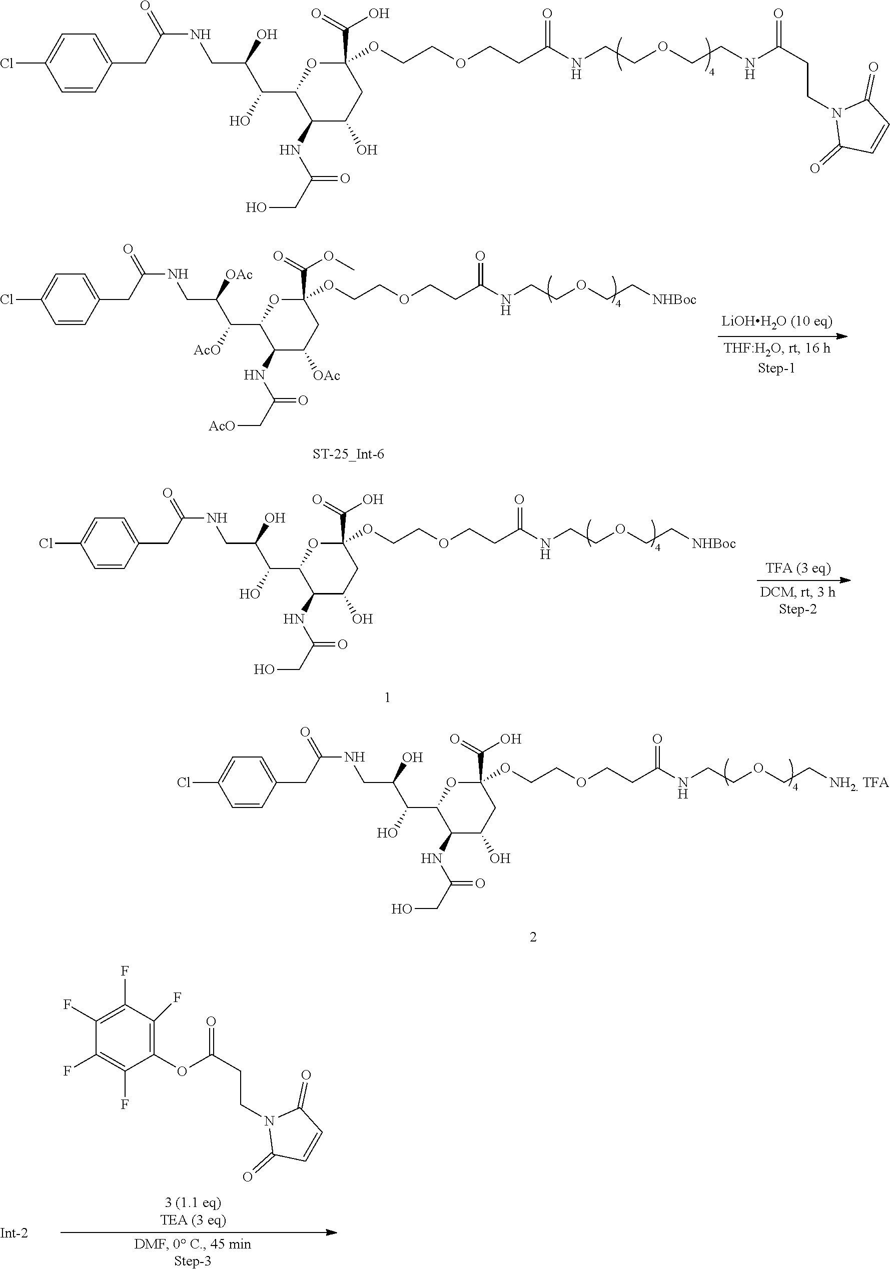

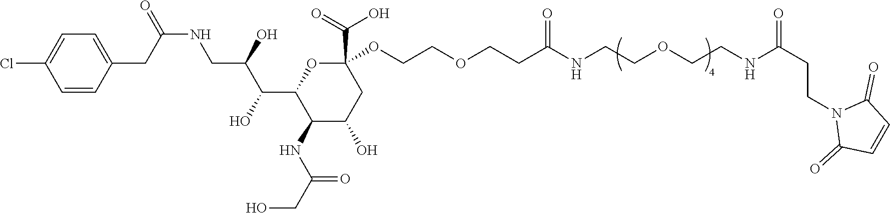

### Synthesis of (2R,4S,5R,6R)-6-((1R,2R)-3-(2-(4-chlorophenyl) acetamido)-1,2-dihydroxypropyl)-2-((2,2-dimethyl-4,21-dioxo-3,8,11,14,17,24-hexaoxa-5,20-diazahexacosan-26-yl) oxy)-4-hydroxy-5-(2-hydroxyacetamido) tetrahydro-2H-pyran-2-carboxylic acid (1)

To a stirred solution of (1R,2R)-1-((2R,3R,4S,6R)-4-acetoxy-3-(2-acetoxyacetamido)-6-((2,2-dimethyl-4,21-dioxo-3,8,11,14,17,24-hexaoxa-5,20-diazahexacosan-26-yl) oxy)-6-(methoxycarbonyl) tetrahydro-2H-pyran-2-yl)-3-(2-(4-chlorophenyl) acetamido) propane-1,2-diyl diacetate (ST-25_Int-6) (0.3 g, 0.31 mmol) in tetrahydrofuran (2.7 mL) was added a solution of lithium hydroxide monohydrate (0.13 mg, 3.12 mmol) in water (0.3 mL) at 0° C. and the reaction mixture was stirred at room temperature for 16 h. After completion the reaction mixture was treated with acidic resin (Dowex H⁺) to pH˜6. The suspension was filtered, and the filtrate was concentrated. The crude residue obtained was purified by C18 reverse phase column chromatography using water and acetonitrile (+0.05%, TFA) as eluents to afford the desired product (2R,4S,5R,6R)-6-((1R,2R)-3-(2-(4-chlorophenyl) acetamido)-1,2-dihydroxypropyl)-2-((2,2-dimethyl-4,21-dioxo-3,8,11,14,17,24-hexaoxa-5,20-diazahexacosan-26-yl) oxy)-4-hydroxy-5-(2-hydroxyacetamido) tetrahydro-2H-pyran-2-carboxylic acid (1) as off white solid. Yield: 0.25 g, Quant. LCMS (ESI±) m/z 911.39 [M+H]⁺.

### Synthesis of (2R,4S,5R,6R)-2-((1-amino-16-oxo-3,6,9,12,19-pentaoxa-15-azahenicosan-21-yl) oxy)-6-((1R,2R)-3-(2-(4-chlorophenyl) acetamido)-1,2-dihydroxypropyl)-4-hydroxy-5-(2-hydroxyacetamido) tetrahydro-2H-pyran-2-carboxylic acid (2)

To a stirred solution of (2R,4S,5R,6R)-6-((1R,2R)-3-(2-([1,1′-biphenyl]-4-yl) acetamido)-1,2-dihydroxypropyl)-2-((2,2-dimethyl-4,21-dioxo-3,8,11,14,17,24-hexaoxa-5,20-diazahexacosan-26-yl) oxy)-4-hydroxy-5-(2-hydroxyacetamido) tetrahydro-2H-pyran-2-carboxylic acid (1) (0.25 g, 0.27 mmol) in anhydrous dichloromethane (2.5 mL) was added trifluoro acetic acid (0.06 ml, 0.82 mmol) at 0° C. The reaction mixture was stirred at room temperature for 2 h. After completion, solvent was concentrated, crude was suspended in methanol and neutralized with tetra alkyl ammonium carbonate, polymer-bound resin. Then the reaction mixture was filtered, and the filtrate was concentrated to afford the desired product (2R,4S,5R,6R)-2-((1-amino-16-oxo-3,6,9,12,19-pentaoxa-15-azahenicosan-21-yl) oxy)-6-((1R,2R)-3-(2-(4-chlorophenyl) acetamido)-1,2-dihydroxypropyl)-4-hydroxy-5-(2-hydroxyacetamido) tetrahydro-2H-pyran-2-carboxylic acid (2) as yellow solid. Yield:200 mg, Crude.

### Synthesis of (2R,4S,5R,6R)-6-((1R,2R)-3-(2-(4-chlorophenyl) acetamido)-1,2-dihydroxypropyl)-2-((25-(2,5-dioxo-2,5-dihydro-1H-pyrrol-1-yl)-6,23-dioxo-3,10,13,16,19-pentaoxa-7,22-diazapentacosyl) oxy)-4-hydroxy-5-(2-hydroxyacetamido) tetrahydro-2H-pyran-2-carboxylic acid (239M)

To a stirred solution (2R,4S,5R,6R)-6-((1R,2R)-3-(2-(4-chlorophenyl)acetamido)-1,2-dihydroxypropyl)-4-hydroxy-5-(2-hydroxyacetamido)-2-((1,1,1-trifluoro-2,19-dioxo-6,9,12,15,22-pentaoxa-314,18-diazatetracosan-24-yl)oxy)tetrahydro-2H-pyran-2-carboxylic acid (2) (200 mg, 0.22 mmol) and 2,5-dioxopyrrolidin-1-yl 3-(2,5-dioxo-2,5-dihydro-1H-pyrrol-1-yl)propanoate (3) (73.78 mg, 0.22 mmol) in N,N-dimethyl formamide (2 mL) was added triethyl amine (0.09 ml, 0.66 mmol) at 0° C. Then the reaction mixture was stirred at room temperature for 45 min. After completion, the reaction mixture was concentrated and purified by C18 prep-HPLC using water and acetonitrile (+0.05%, TFA) as eluents to afford the desired product (2R,4S,5R,6R)-6-((1R,2R)-3-(2-(4-chlorophenyl) acetamido)-1,2-dihydroxypropyl)-2-((25-(2,5-dioxo-2,5-dihydro-1H-pyrrol-1-yl)-6,23-dioxo-3,10,13,16,19-pentaoxa-7,22-diazapentacosyl) oxy)-4-hydroxy-5-(2-hydroxyacetamido) tetrahydro-2H-pyran-2-carboxylic acid (239M) as white solid. Yield: 40 mg, 19%; LCMS (ESI±) m/z 962.40 [M+H]⁺. ¹H NMR (400 MHz, MeOD-d4) δ 7.32 (t, J=1.7 Hz, 4H), 6.85 (s, 2H), 4.05 (d, J=2.8 Hz, 2H), 3.95-3.89 (m, 5H), 3.85 (dd, J=17.8, 8.8 Hz, 3H), 3.81-3.49 (m, 25H), 3.44-3.37 (m, 3H), 3.35-3.21 (m, 1H), 2.75 (dd, J=12.7, 4.1 Hz, 1H), 2.49 (td, J=6.5, 4.1 Hz, 4H), 1.78 (t, J=12.0 Hz, 1H).

### (2R,4S,5R,6R)-6-((1R,2R)-3-(2-(4-chlorophenyl) acetamido)-1,2-dihydroxypropyl)-2-((6-((3,4-dioxo-2-((2-oxo-6,9,12,15-tetraoxa-3-azaheptadecan-17-yl)amino) cyclobut-1-en-1-yl) amino)hexyl)oxy)-4-hydroxy-5-(2-hydroxyacetamido) tetrahydro-2H-pyran-2-carboxylic acid (Cpd. 240)

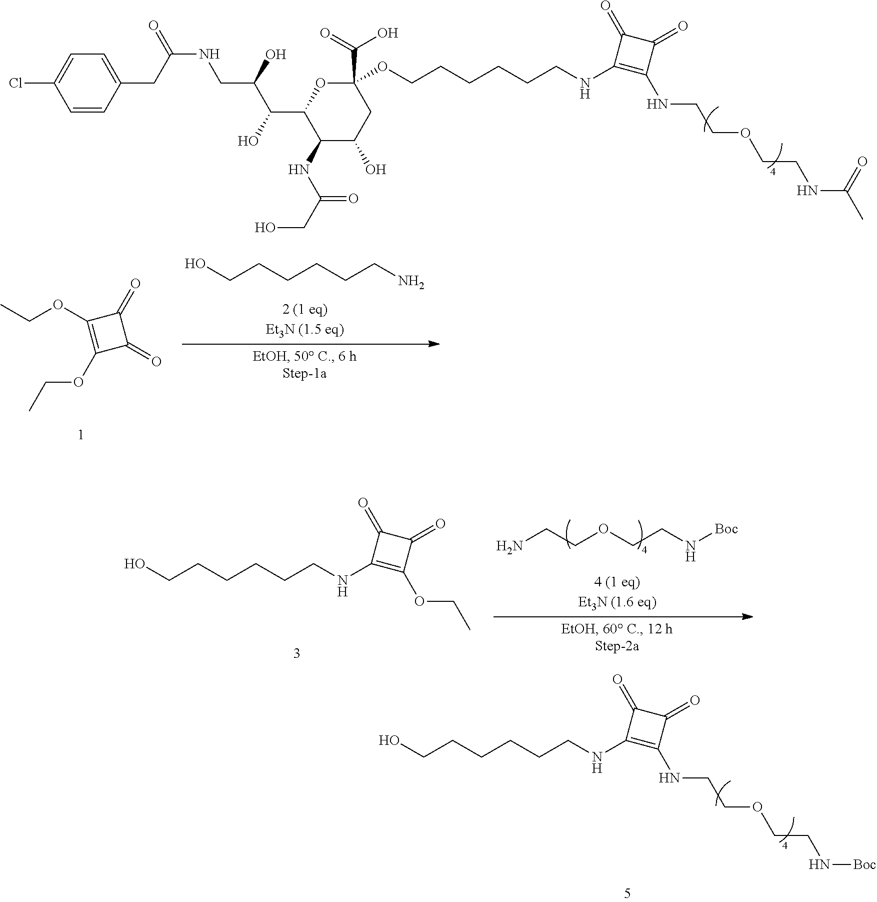

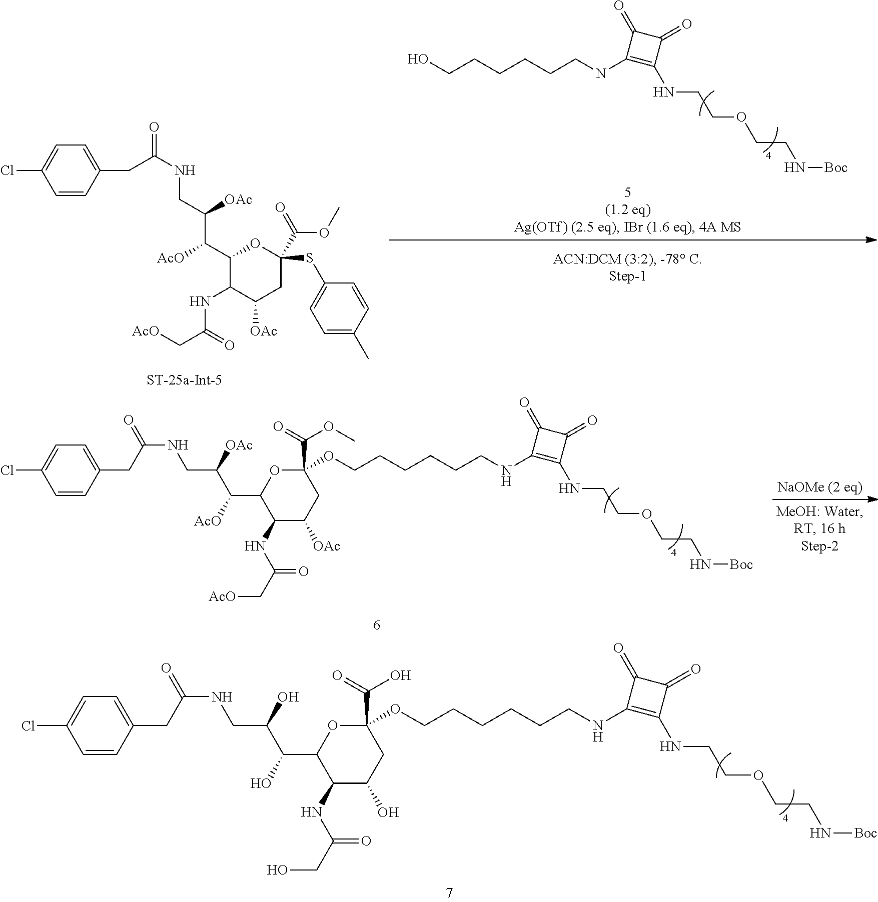

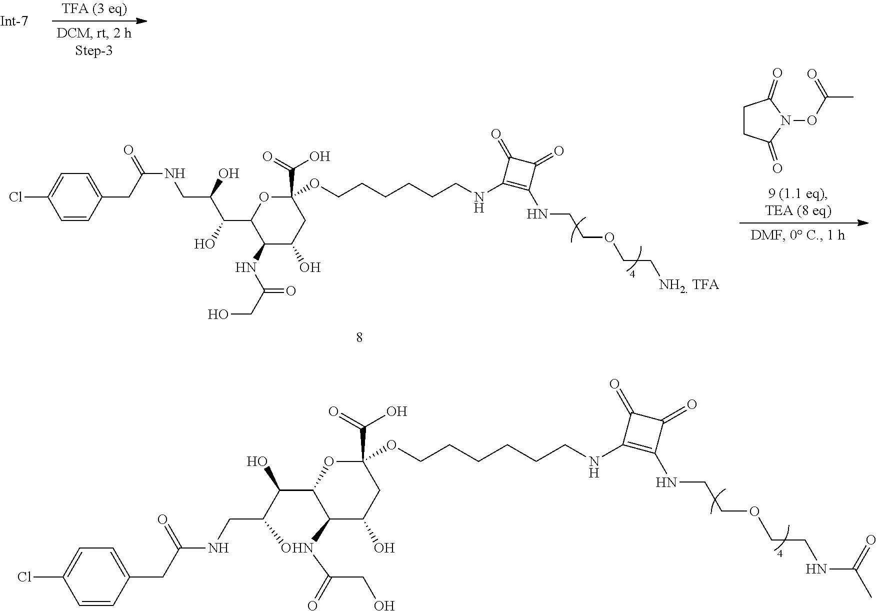

### Synthesis of 3-ethoxy-4-((6-hydroxyhexyl)amino) cyclobut-3-ene-1,2-dione (3)

To a stirred solution of 3,4-diethoxycyclobut-3-ene-1,2-dione (1) (5 g, 29.41 mmol) and 6-aminohexan-1-ol (2) (1.72 g, 14.70 mmol) in ethanol (40 mL) was added triethyl amine (6.36 ml, 44.11 mmol) dropwise at room temperature and heated at 50° C. for 6 h. After completion, the reaction mixture was concentrated, the crude residue was purified by silica gel column chromatography using methanol in dichloromethane as eluents to afford 3-ethoxy-4-((6-hydroxyhexyl)amino) cyclobut-3-ene-1,2-dione as a colorless liquid. Yield: 5 g, 71%, LCMS (ESI±) m/z 242.2 [M+H]⁺.

### Synthesis of tert-butyl (14-((2-((6-hydroxyhexyl)amino)-3,4-dioxocyclobut-1-en-1-yl)amino)-3,6,9,12-tetraoxatetradecyl) carbamate (5)

To a stirred solution of 3-ethoxy-4-((6-hydroxyhexyl)amino)cyclobut-3-ene-1,2-dione (3) (5 g, 20.72 mmol) in ethanol (50 mL) and tert-butyl (14-amino-3,6,9,12-tetraoxatetradecyl)carbamate (4) (6.97 g, 20.72 mmol) at room temperature. After 10 min triethylamine (4.49 ml, 31.12 mmol) was added at 0° C. The reaction mixture was stirred at 60° C. for 6 h. After completion, the reaction mixture was concentrated, the crude residue was purified by C18 reverse phase column chromatography using acetonitrile and water as eluents (+0.05%, TFA) to afford tert-butyl (14-((2-((6-hydroxyhexyl)amino)-3,4-dioxocyclobut-1-en-1-yl)amino)-3,6,9,12-tetraoxatetradecyl) carbamate as pale-yellow liquid. Yield: 3 g, 27%, LCMS (ESI±) m/z 532.4 [M+H]⁺.

### Synthesis of (1R,2R)-1-((2R,3R,4S,6R)-4-acetoxy-3-(2-acetoxyacetamido)-6-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-6-(methoxycarbonyl) tetrahydro-2H-pyran-2-yl)-3-(2-(4-chlorophenyl) acetamido) propane-1,2-diyl diacetate (6)

To a stirred solution of (1R,2R)-1-((2R,3R,4S,6R)-4-acetoxy-3-(2-fluoroacetamido)-6-(methoxycarbonyl)-6-(p-tolylthio)tetrahydro-2H-pyran-2-yl)-3-(2-(4-chlorophenyl)acetamido)propane-1,2-diyl diacetate (239_Int-5) (2 g, 2.61 mmol) and tert-butyl (14-((2-((6-hydroxyhexyl)amino)-3,4-dioxocyclobut-1-en-1-yl)amino)-3,6,9,12-tetraoxatetradecyl)carbamate (5) (1.66 g, 3.14 mmol), Silver (+1) trifluoro methane sulfonate Ag(OTf) (1.68 g, 6.54 mmol) and activated 4 Å powdered molecular sieves (5 g) in anhydrous dichloromethane (12 mL) and anhydrous acetonitrile (24 mL) was stirred at room temperature for 1 h under nitrogen atmosphere. The solution was cooled to −78° C. and a solution of iodine monobromide (0.81 g, 3.92 mmol) in dichloromethane (4 ml) was added dropwise. The reaction mixture was stirred at the same temperature for 2 h. After completion, the reaction mixture was quenched by addition of triethylamine (5 mL) and stirred at room temperature. The reaction mixture was filtered, and the filtrate was washed by saturated aqueous sodium thiosulfate, dried over sodium sulfate, filtered, and concentrated. The crude residue was purified by C18 reverse phase column chromatography using acetonitrile and water (0.05% TFA) as eluents to afford the desired product (1R,2R)-1-((2R,3R,4S,6R)-4-acetoxy-3-(2-acetoxyacetamido)-6-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-6-(methoxycarbonyl) tetrahydro-2H-pyran-2-yl)-3-(2-(4-chlorophenyl) acetamido) propane-1,2-diyl diacetate (6) as yellow solid. Yield: 1 g, 43%, LCMS (ESI±) m/z 933.0 [M+H]⁺.

### Synthesis of (2R,4S,5R,6R)-6-((1R,2R)-3-(2-(4-chlorophenyl) acetamido)-1,2-dihydroxypropyl)-2-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-4-hydroxy-5-(2-hydroxyacetamido) tetrahydro-2H-pyran-2-carboxylic acid (7)

To a stirred solution of (1R,2R)-1-((2R,3R,4S,6R)-4-acetoxy-3-(2-acetoxyacetamido)-6-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-6-(methoxycarbonyl) tetrahydro-2H-pyran-2-yl)-3-(2-(4-chlorophenyl) acetamido) propane-1,2-diyl diacetate (6) (0.5 g, 0.42 mmol) in methanol (7 mL), was added a solution of sodium methoxide 25% in methanol (0.04 mL, 0.85 mmol) in water (1 mL). The reaction mixture was stirred at room temperature for 6 h then treated with Dowex 50 resin to reach pH ˜6. The suspension was filtered, and the filtrate was concentrated. The residue obtained was purified by C18 reverse phase column chromatography using acetonitrile and water (0.05% TFA) as eluents to afford the desired product (2R,4S,5R,6R)-6-((1R,2R)-3-(2-(4-chlorophenyl) acetamido)-1,2-dihydroxypropyl)-2-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-4-hydroxy-5-(2-hydroxyacetamido) tetrahydro-2H-pyran-2-carboxylic acid (7) as off white solid. Yield: 0.31 g, 74%; LCMS m/z 693.45 [M+H]⁺.

### Synthesis of (2R,4S,5R,6R)-6-((1R,2R)-3-(2-(4-chlorophenyl) acetamido)-1,2-dihydroxypropyl)-2-((6-((3,4-dioxo-2-((1,1,1-trifluoro-2-oxo-6,9,12,15-tetraoxa-314-azaheptadecan-17-yl)amino) cyclobut-1-en-1-yl)amino)hexyl)oxy)-4-hydroxy-5-(2-hydroxyacetamido) tetrahydro-2H-pyran-2-carboxylic acid (8)

To a stirred solution of (2R,4S,5R,6R)-6-((1R,2R)-3-(2-(4-chlorophenyl) acetamido)-1,2-dihydroxypropyl)-2-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-4-hydroxy-5-(2-hydroxyacetamido) tetrahydro-2H-pyran-2-carboxylic acid (7) (0.3 g, 0.3 mmol) in dichloromethane (5 mL), was added trifluoro acetic acid (0.5 mL) at 0° C. The reaction mixture was stirred at room temperature for 2 h. After completion, the reaction mixture was concentrated and the residue was purified by C18 reverse phase column chromatography using acetonitrile and water (0.05% TFA) as eluents to afford the desired product (2R,4S,5R,6R)-6-((1R,2R)-3-(2-(4-chlorophenyl) acetamido)-1,2-dihydroxypropyl)-2-((6-((3,4-dioxo-2-((1,1,1-trifluoro-2-oxo-6,9,12,15-tetraoxa-314-azaheptadecan-17-yl)amino) cyclobut-1-en-1-yl)amino)hexyl)oxy)-4-hydroxy-5-(2-hydroxyacetamido) tetrahydro-2H-pyran-2-carboxylic acid (8) as brown semi solid product. Yield: 0.21 g, 76%; LCMS m/z 888.8 [M−H]⁻.

### Synthesis of (2R,4S,5R,6R)-6-((1R,2R)-3-(2-(4-chlorophenyl) acetamido)-1,2-dihydroxypropyl)-2-((6-((3,4-dioxo-2-((2-oxo-6,9,12,15-tetraoxa-3-azaheptadecan-17-yl)amino) cyclobut-1-en-1-yl)amino)hexyl)oxy)-4-hydroxy-5-(2-hydroxyacetamido) tetrahydro-2H-pyran-2-carboxylic acid (240)

To a stirred solution of (2R,4S,5R,6R)-6-((1R,2R)-3-(2-(4-chlorophenyl)acetamido)-1,2-dihydroxypropyl)-2-((6-((3,4-dioxo-2-((1,1,1-trifluoro-2-oxo-6,9,12,15-tetraoxa-314-azaheptadecan-17-yl)amino)cyclobut-1-en-1-yl)amino)hexyl)oxy)-4-hydroxy-5-(2-hydroxyacetamido)tetrahydro-2H-pyran-2-carboxylic acid (8) (100 mg, 0.10 mmol) in N,N-Dimethyl formamide (1 mL) were added 2,5-dioxopyrrolidin-1-yl acetate (9) (17.49 mg, 0.11 mmol, 1.1 eq) and triethylamine (0.04 ml, 0.30 mmol, 3eq) at 0° C. The reaction mixture was stirred at room temperature for 1 h. After completion, the reaction mixture was concentrated and purified by C18 prep-HPLC using acetonitrile and water (+0.05% TFA) as eluents to afford the desired product (2R,4S,5R,6R)-6-((1R,2R)-3-(2-(4-chlorophenyl) acetamido)-1,2-dihydroxypropyl)-2-((6-((3,4-dioxo-2-((2-oxo-6,9,12,15-tetraoxa-3-azaheptadecan-17-yl) amino) cyclobut-1-en-1-yl)amino)hexyl)oxy)-4-hydroxy-5-(2-hydroxyacetamido) tetrahydro-2H-pyran-2-carboxylic acid as white solid. Yield: 11 mg, 11.65%, LCMS (ESI) m/z 933.0 [M+2]⁺. ¹H NMR (400 MHz, MeOD) δ 7.32 (s, 4H), 4.05 (s, 2H), 3.86-3.72 (m, 6H), 3.66 (dd, J=7.6, 4.5 Hz, 15H), 3.60-3.52 (m, 5H), 3.47-3.35 (m, 4H), 2.82 (dd, J=12.8 Hz, 1H)1.97 (s, 3H), 1.74 (t, J=11.8 Hz, 1H), 1.65-1.57 (m, 4H), 1.41 (s, 4H), 1.32 (s, 3H).

### (2R,4S,5R,6R)-6-((1R,2R)-3-(2-(4-chlorophenyl) acetamido)-1,2-dihydroxypropyl)-2-((6-((2-((18-(2,5-dioxo-2,5-dihydro-1H-pyrrol-1-yl)-16-oxo-3,6,9,12-tetraoxa-15-azaoctadecyl) amino)-3,4-dioxocyclobut-1-en-1-yl)amino)hexyl)oxy)-4-hydroxy-5-(2-hydroxyacetamido) tetrahydro-2H-pyran-2-carboxylic acid (Cpd. 240M)

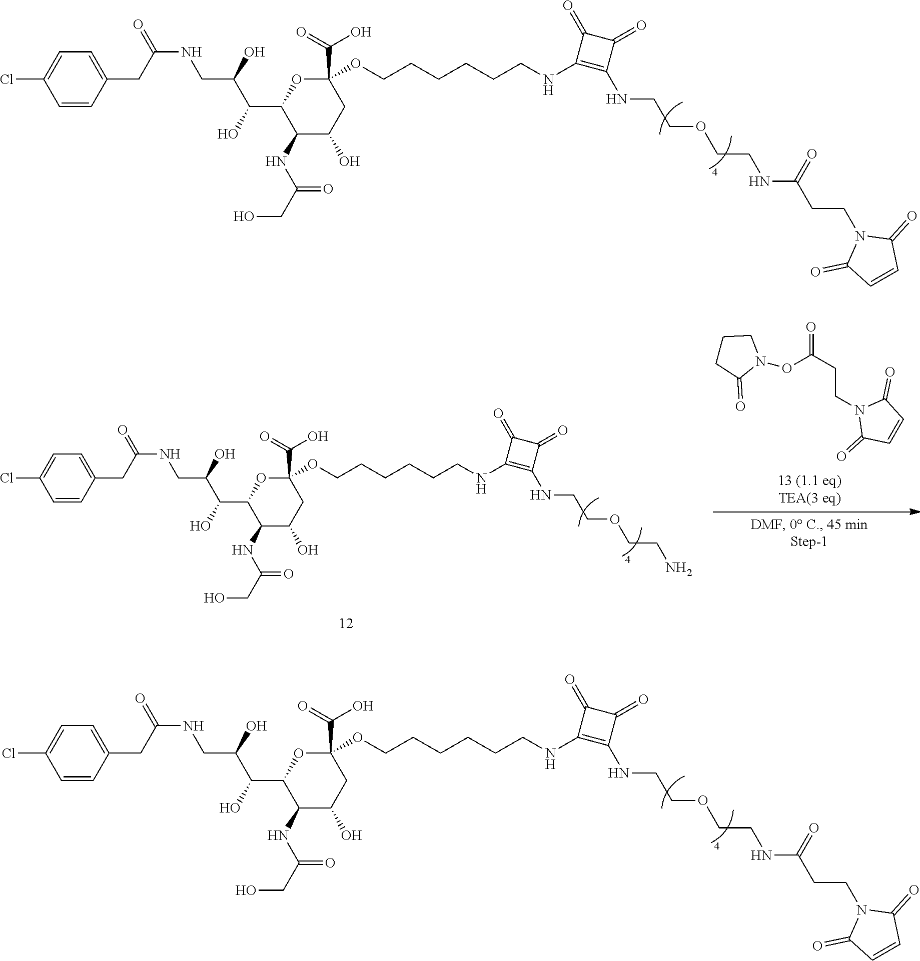

### Synthesis of (2R,4S,5R,6R)-2-((6-((2-((14-amino-3,6,9,2-tetraoxatetradecyl)amino)-3,4-dioxocyclobut-1-en-1-yl)amino)hexyl)oxy)-6-((1R,2R)-3-(2-(4-chlorophenyl) acetamido)-1,2-dihydroxypropyl)-4-hydroxy-5-(2-hydroxyacetamido) tetrahydro-2H-pyran-2-carboxylic acid (240M)

To a stirred solution of (2R,4S,5R,6R)-2-((6-((2-((14-amino-3,6,9,12-tetraoxatetradecyl)amino)-3,4-dioxocyclobut-1-en-1-yl)amino)hexyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (240_Int-8) (150 mg, 0.16 mmol) in N,N-Dimethyl formamide (1 mL) were added 2-oxopyrrolidin-1-yl 3-(2,5-dioxo-2,5-dihydro-1H-pyrrol-1-yl)propanoate (1) (46.77 mg, 0.18 mmol) and triethylamine (0.07 ml, 0.5 mmol) at 0° C. The reaction mixture was stirred at the same temperature for 45 min. After completion, the reaction mixture was concentrated and purified by C18 prep-HPLC using acetonitrile and water (+0.05% TFA) as eluents to afford the desired product (2R,4S,5R,6R)-2-((6-((2-((14-amino-3,6,9,12-tetraoxatetradecyl)amino)-3,4-dioxocyclobut-1-en-1-yl)amino)hexyl)oxy)-6-((1R,2R)-3-(2-(4-chlorophenyl) acetamido)-1,2-dihydroxypropyl)-4-hydroxy-5-(2-hydroxyacetamido) tetrahydro-2H-pyran-2-carboxylic acid (240M) as white solid. Yield: 3 mg, 0.01%, LCMS (ESI) m/z 933.0 [M+H]⁺. ¹H NMR (400 MHz, MeOD) δ 7.31 (s, 4H), 6.83 (s, 2H), 4.05 (s, 2H), 3.91-3.72 (m, 9H), 3.69-3.52 (m, 22H), 3.43-3.37 (m, 2H), 2.82 (dd, J=12.8 Hz, 1H), 2.44 (t, J=6.8, 3H), 1.74 (t, J=11.8 Hz, 1H), 1.65-1.57 (m, 4H), 1.41 (s, 4H).

### (2R,4S,5R,6R)-6-((1R,2R)-3-(2-(4-chlorophenyl) acetamido)-1,2-dihydroxypropyl)-2-((2,18-dioxo-6,9,12,15-tetraoxa-3,19-diazatetracosan-24-yl) oxy)-4-hydroxy-5-(2-hydroxyacetamido) tetrahydro-2H-pyran-2-carboxylic acid (Cpd. 241)

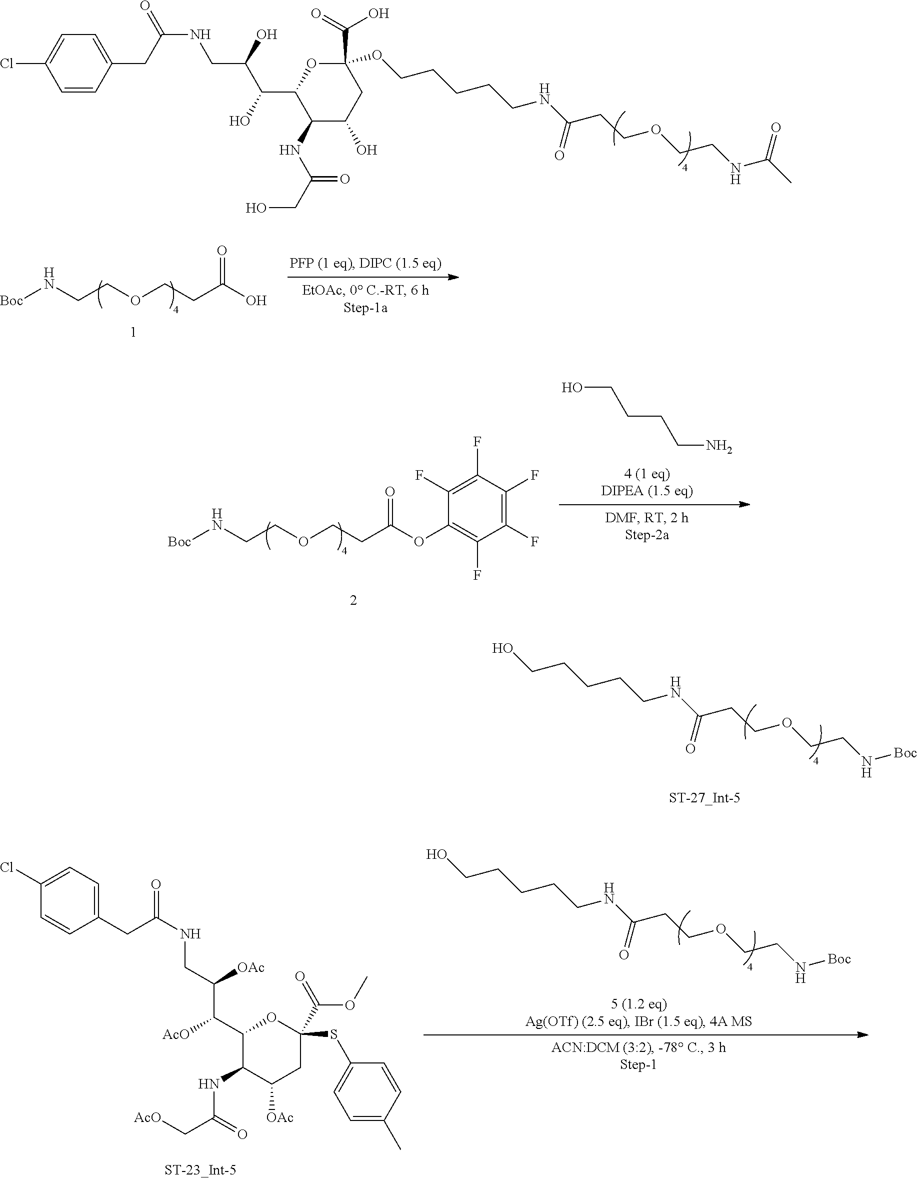

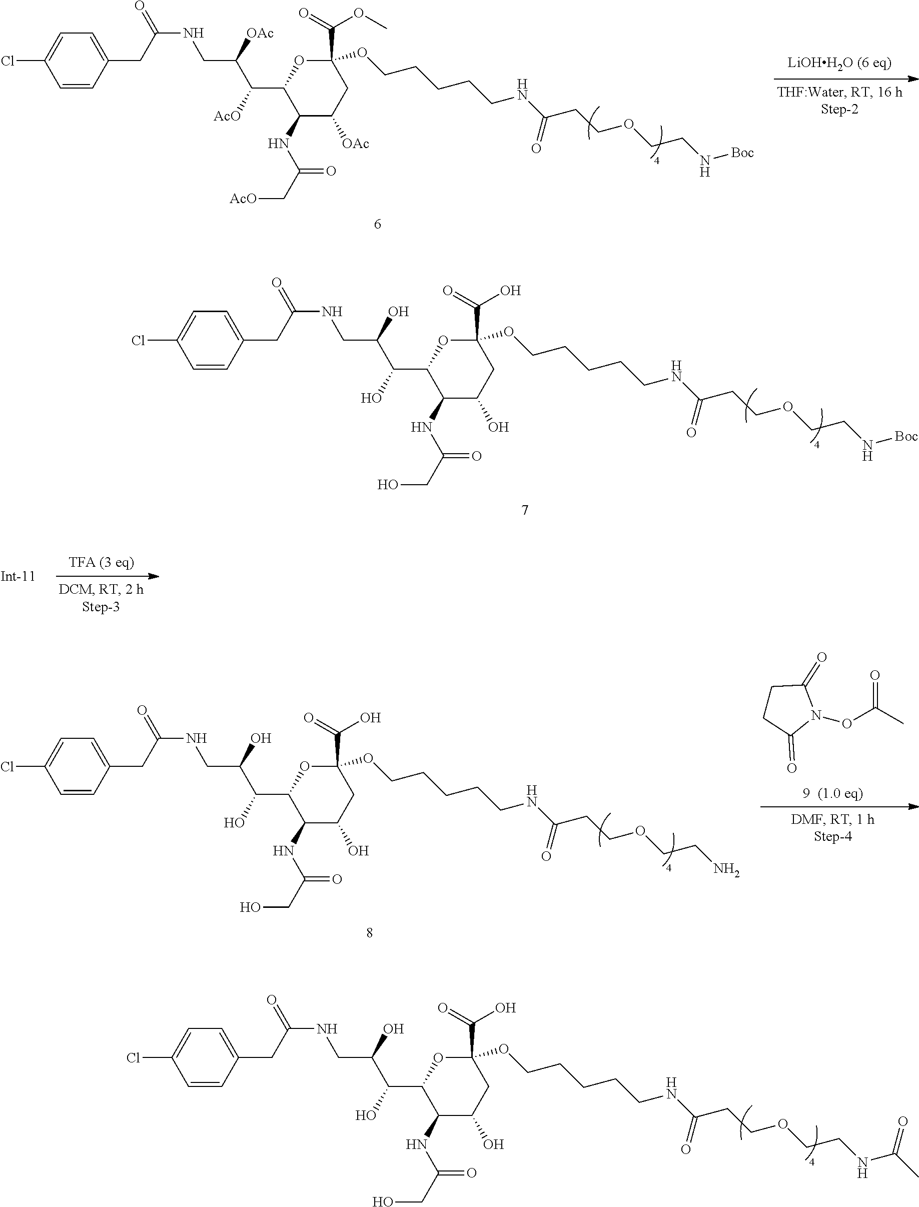

### Synthesis of perfluorophenyl 2,2-dimethyl-4-oxo-3,8,11,14,17-pentaoxa-5-azaicosan-20-oate (2)

To a stirred solution of 2,2-dimethyl-4-oxo-3,8,11,14,17-pentaoxa-5-azaicosan-20-oic acid(1) (5 g, 13.6 mmol) and penta-fluoro phenol (2.5 g, 13.6 mmol) in dry ethyl acetate (50 mL) was added N, N-di-isopropyl carbodiimide (2.58 g, 20.4 mmol) dropwise at 0° C. The reaction mixture was stirred at room temperature for next 6 h. After completion, the reaction mixture was filtered, and the filtrate was concentrated. The crude residue was purified by silica gel column chromatography, by using ethyl acetate and hexane as eluents to afford perfluorophenyl 2,2-dimethyl-4-oxo-3,8,11,14,17-pentaoxa-5-azaicosan-20-oate (2) as off-white solid. Yield: 5 g, 69%; LCMS (ESI±) m/z 533.6 [M+H]⁺.

### Synthesis of tert-butyl (21-hydroxy-15-oxo-3,6,9,12-tetraoxa-16-azahenicosyl) carbamate (ST-27_Int-5)

To a stirred solution of perfluorophenyl 2,2-dimethyl-4-oxo-3,8,11,14,17-pentaoxa-5-azaicosan-20-oate (2) (4 g, 7.52 mmol) and 4-aminobutan-1-ol (4) (3.11 g, 6.77 mmol) in N,N-dimethyl formamide (1 mL) was added triethylamine (3.25 ml, 22.58 mmol) at 0° C. The resulting reaction mixture was stirred at room temperature for 2 h. After completion, the reaction mixture was concentrated and purified by C18 reverse phase chromatography using water and acetonitrile (+0.05%, TFA) as eluents to afford tert-butyl (21-hydroxy-15-oxo-3,6,9,12-tetraoxa-16-azahenicosyl) carbamate (ST-27_Int-5) as semi-solid compound. Yield: 3 g; quant. ¹H NMR (400 MHz, DMSO-d6): δ 7.79 (t, J=5.2. 1H), 6.75 (t, J=5.6, 1H), 3.6 (t, J=6.4, 2H), 3.50-3.48 (m, 12H), 3.39-3.31 (m, 4H), 3.17-2.99 (m, 4H), 2.29(t, J=6.4, 2H), 1.44-1.36 (m, 13H), 1.20-1.14 (m, 3H).

### Synthesis of (1R,2R)-1-((2R,3R,4S,6R)-4-acetoxy-3-(2-acetoxyacetamido)-6-((2,2-dimethyl-4,20-dioxo-3,8,11,14,17-pentaoxa-5,21-diazahexacosan-26-yl) oxy)-6-(methoxycarbonyl) tetrahydro-2H-pyran-2-yl)-3-(2-(4-chlorophenyl) acetamido) propane-1,2-diyl diacetate (6)

To a stirred solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (ST-23_Int-5) (1 g, 1.30 mmol), tert-butyl (21-hydroxy-15-oxo-3,6,9,12-tetraoxa-16-azahenicosyl)carbamate (ST-27_Int-5) (0.88 g, 1.96 mmol) in anhydrous acetonitrile (9 mL) and anhydrous dichloromethane (6 mL) were added 4 Å powdered molecular sieves (4 g) and Ag(OTf) (0.50 g, 1.96 mmol) under argon atmosphere. The reaction mixture was stirred for 30 min at room temperature, then cooled to −78° C. and a solution of iodine monobromide (0.67 g, 3.26 mmol) in dichloromethane (4 mL) was added dropwise at the same temperature. The reaction mixture was stirred for 2 h. After completion, the reaction mixture was diluted with dichloromethane (15 ml), quenched with triethylamine (up to pH=7), filtered, then the filtrate was washed with 10% aqueous sodium thiosulfate solution, dried over sodium sulfate, and concentrated. The crude residue was purified by C18 reverse phase column chromatography using water and acetonitrile (0.05%, TFA) as eluents to afford (1R,2R)-1-((2R,3R,4S,6R)-4-acetoxy-3-(2-acetoxyacetamido)-6-((2,2-dimethyl-4,20-dioxo-3,8,11,14,17-pentaoxa-5,21-diazahexacosan-26-yl) oxy)-6-(methoxycarbonyl) tetrahydro-2H-pyran-2-yl)-3-(2-(4-chlorophenyl) acetamido) propane-1,2-diyl diacetate (6) as yellow solid. Yield: (0.8 g, 56%); LCMS (ESI±) m/z 1091.60 [M+H]⁺.

### Synthesis of (2R,4S,5R,6R)-6-((1R,2R)-3-(2-(4-chlorophenyl) acetamido)-1,2-dihydroxypropyl)-2-((2,2-dimethyl-4,20-dioxo-3,8,11,14,17-pentaoxa-5,21-diazahexacosan-26-yl) oxy)-4-hydroxy-5-(2-hydroxyacetamido) tetrahydro-2H-pyran-2-carboxylic acid (7)

To a stirred solution of (1R,2R)-1-((2R,3R,4S,6R)-4-acetoxy-3-(2-acetoxyacetamido)-6-((2,2-dimethyl-4,20-dioxo-3,8,11,14,17-pentaoxa-5,21-diazahexacosan-26-yl) oxy)-6-(methoxycarbonyl) tetrahydro-2H-pyran-2-yl)-3-(2-(4-chlorophenyl) acetamido) propane-1,2-diyl diacetate (6) (0.8 g, 0.73 mmol) in methanol (7 mL) and water (3 mL) was added solution of lithium hydroxide monohydrate (0.17 g, 0.73 mmol) in water (3 mL) at 0° C. and the reaction mixture was stirred at room temperature for 16 h. After completion the reaction mixture was treated with acidic resin (Dowex H⁺) to pH˜6 and the suspension was filtered. The filtrate was concentrated to get the crude residue was purified Prep HPLC by using water and acetonitrile (+0.05% of TFA) as eluents to afford (2R,4S,5R,6R)-6-((1R,2R)-3-(2-(4-chlorophenyl) acetamido)-1,2-dihydroxypropyl)-2-((2,2-dimethyl-4,20-dioxo-3,8,11,14,17-pentaoxa-5,21-diazahexacosan-26-yl) oxy)-4-hydroxy-5-(2-hydroxyacetamido) tetrahydro-2H-pyran-2-carboxylic acid (7) as off white solid. Yield: 0.5 g, 55%; LCMS (ESI±) m/z 907.2 [M−H]⁻.

### Synthesis of (2R,4S,5R,6R)-2-((1-amino-15-oxo-3,6,9,12-tetraoxa-16-azahenicosan-21-yl) oxy)-6-((1R,2R)-3-(2-(4-chlorophenyl) acetamido)-1,2-dihydroxypropyl)-4-hydroxy-5-(2-hydroxyacetamido) tetrahydro-2H-pyran-2-carboxylic acid (8)

To a stirred solution of (2R,4S,5R,6R)-6-((1R,2R)-3-(2-(4-chlorophenyl) acetamido)-1,2-dihydroxypropyl)-2-((2,2-dimethyl-4,20-dioxo-3,8,11,14,17-pentaoxa-5,21-diazahexacosan-26-yl) oxy)-4-hydroxy-5-(2-hydroxyacetamido) tetrahydro-2H-pyran-2-carboxylic acid (7) (0.3 g, 0.2 mmol) in anhydrous dichloromethane (1 mL) was added trifluoro acetic acid (1 mL) at 0° C. The reaction mixture was stirred at room temperature for 2 h. After completion, the mixture was concentrated and the crude material was suspended in methanol and neutralized with tetra alkyl ammonium carbonate polymer-supported and filtered. The filtrate was concentrated to afford (2R,4S,5R,6R)-2-((1-amino-15-oxo-3,6,9,12-tetraoxa-16-azahenicosan-21-yl) oxy)-6-((1R,2R)-3-(2-(4-chlorophenyl) acetamido)-1,2-dihydroxypropyl)-4-hydroxy-5-(2-hydroxyacetamido) tetrahydro-2H-pyran-2-carboxylic acid (8) as yellow solid. Yield:300 mg (Crude); LCMS (ESI±) m/z 809.30 [M+H]⁺.

### Synthesis of (2R,4S,5R,6R)-6-((1R,2R)-3-(2-(4-chlorophenyl) acetamido)-1,2-dihydroxypropyl)-2-((2,18-dioxo-6,9,12,15-tetraoxa-3,19-diazatetracosan-24-yl) oxy)-4-hydroxy-5-(2-hydroxyacetamido) tetrahydro-2H-pyran-2-carboxylic acid (241)

To a stirred solution of (2R,4S,5R,6R)-2-((1-amino-15-oxo-3,6,9,12-tetraoxa-16-azahenicosan-21-yl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (8) (100 mg, 0.12 mmol) in N,N-Dimethyl formamide (1 mL) was added 2,5-dioxopyrrolidin-1-yl acetate (10) (19.4 mg, 0.12 mmol) at 0° C. The resulting reaction mixture was stirred at room temperature for 1 h. After completion, the reaction mixture was purified by C18 prep-HPLC using water and acetonitrile (0.05%, TFA) as eluents to afford (2R,4S,5R,6R)-6-((1R,2R)-3-(2-(4-chlorophenyl) acetamido)-1,2-dihydroxypropyl)-2-((2,18-dioxo-6,9,12,15-tetraoxa-3,19-diazatetracosan-24-yl) oxy)-4-hydroxy-5-(2-hydroxyacetamido) tetrahydro-2H-pyran-2-carboxylic acid (241) as white solid. Yield: 22 mg, 21%; LCMS (ESI±) m/z 851.34 [M+H]⁺. ¹H NMR (400 MHz, MeOD-d4) δ 7.32 (t, J=1.7 Hz, 4H), 4.04 (s, 2H), 3.93-3.83 (m, 4H), 3.83-3.74 (m, 3H), 3.72 (dd, J=11.3, 3.3 Hz, 12H), 3.65 (dtd, J=11.3, 3.4, 1.8 Hz, 4H), 3.60-3.52 (m, 2H), 3.44-3.39 (m, 3H), 3.35-3.31 (m, 1H), 3.31-3.15 (m, 2H), 2.74 (dd, J=12.3, 4.0 Hz, 1H), 2.46 (t, J=6.1 Hz, 2H), 2.00-1.94 (m, 3H), 1.75 (t, J=11.9 Hz, 1H), 1.54-1.46 (m, 4H), 1.41 (t, J=7.0 Hz, 2H).

### (2R,4S,5R,6R)-6-((1R,2R)-3-(2-(4-chlorophenyl) acetamido)-1,2-dihydroxypropyl)-2-((1-(2,5-dioxo-2,5-dihydro-1H-pyrrol-1-yl)-3,19-dioxo-7,10,13,16-tetraoxa-4,20-diazapentacosan-25-yl) oxy)-4-hydroxy-5-(2-hydroxyacetamido) tetrahydro-2H-pyran-2-carboxylic acid (Cpd. 241M)

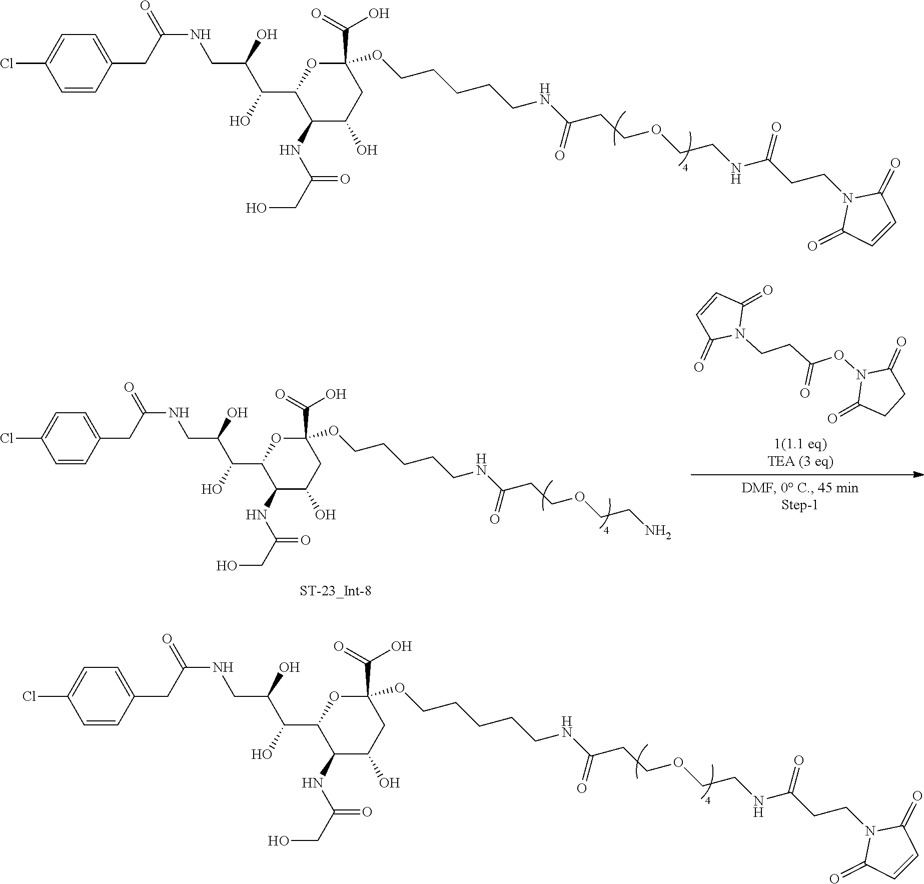

### Synthesis of (2R,4S,5R,6R)-6-((1R,2R)-3-(2-(4-chlorophenyl) acetamido)-1,2-dihydroxypropyl)-2-((1-(2,5-dioxo-2,5-dihydro-1H-pyrrol-1-yl)-3,19-dioxo-7,10,13,16-tetraoxa-4,20-diazapentacosan-25-yl) oxy)-4-hydroxy-5-(2-hydroxyacetamido) tetrahydro-2H-pyran-2-carboxylic acid (241M)

To a stirred solution of (2R,4S,5R,6R)-2-((1-amino-15-oxo-3,6,9,12-tetraoxa-16-azahenicosan-21-yl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (ST-23_Int-5) (150 mg, 0.18 mmol) in N,N-Dimethyl formamide (1.5 mL) were added 2,5-dioxopyrrolidin-1-yl 3-(2,5-dioxo-2,5-dihydro-1H-pyrrol-1-yl)propanoate (1) (49.3 mg, 0.18 mmol) and (0.08 ml, 0.55 mmol) at 0° C. The resulting reaction mixture was stirred at room temperature for 1 h. After completion, the reaction mixture was purified by C18 prep-HPLC using water and acetonitrile (+0.05%, TFA) as eluents to afford (2R,4S,5R,6R)-6-((1R,2R)-3-(2-(4-chlorophenyl) acetamido)-1,2-dihydroxypropyl)-2-((1-(2,5-dioxo-2,5-dihydro-1H-pyrrol-1-yl)-3,19-dioxo-7,10,13,16-tetraoxa-4,20-diazapentacosan-25-yl) oxy)-4-hydroxy-5-(2-hydroxyacetamido) tetrahydro-2H-pyran-2-carboxylic acid (241M) as white solid. Yield: 18 mg, 10%; LCMS (ESI±) m/z 960.70 [M+H]⁺. ¹H NMR (400 MHz, MeOD) δ 7.90 (d, J=7.8 Hz, 1H), 7.31 (s, 4H), 6.83 (s, 2H), 4.03 (s, 2H), 3.91-3.67 (m, 8H), 3.66-3.59 (m, 10H), 3.57-3.48 (m, 5H), 3.45-3.35 (m, 3H), 3.29-3.14 (m, 2H), 2.74 (dd, J=12.3, 4.0 Hz, 1H), 2.46 (dt, J=12.7, 6.5 Hz, 4H), 1.74 (t, J=11.9 Hz, 1H), 1.54 (dt, J=26.9, 7.0 Hz, 4H), 1.44-1.36 (m, 4H).

### (2R,4S,5R,6R)-6-((1R,2R)-3-(2-([1,1′-biphenyl]-4-yl)acetamido)-1,2-dihydroxypropyl)-2-(2-(2-(2-((2-((2-(2-acetamidoethoxy)ethyl)amino)-3,4-dioxocyclobut-1-en-1-yl)amino)ethoxy)ethoxy)ethoxy)-4-hydroxy-5-(2-hydroxyacetamido)tetrahydro-2H-pyran-2-carboxylic acid (Cpd. 242)

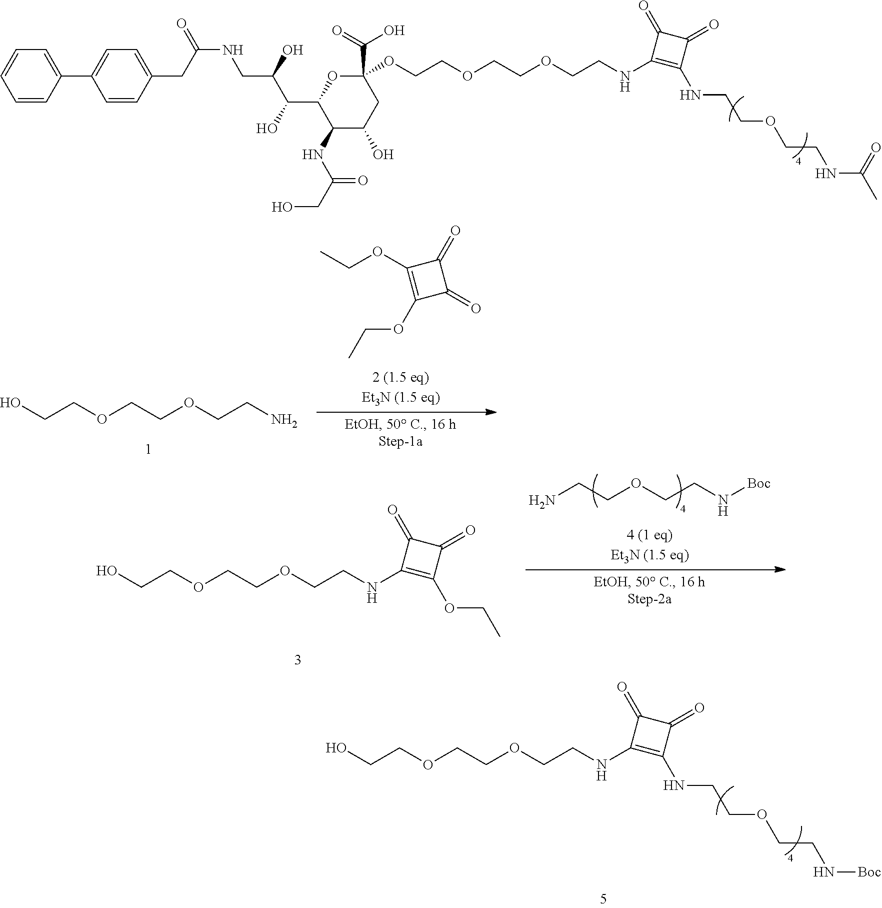

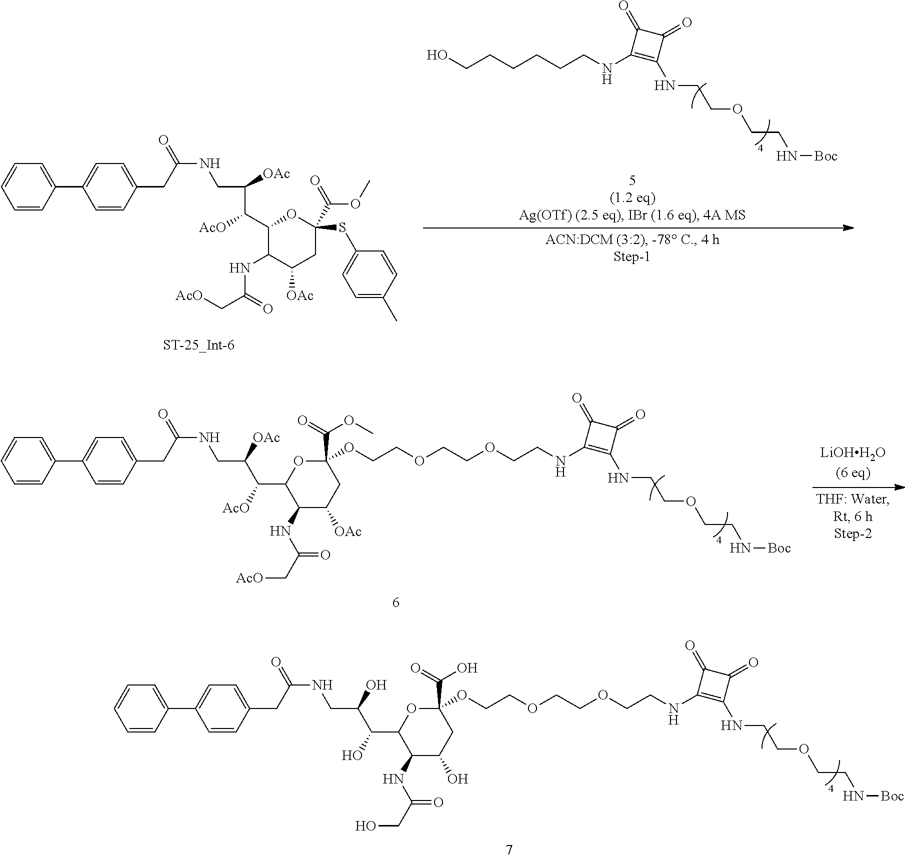

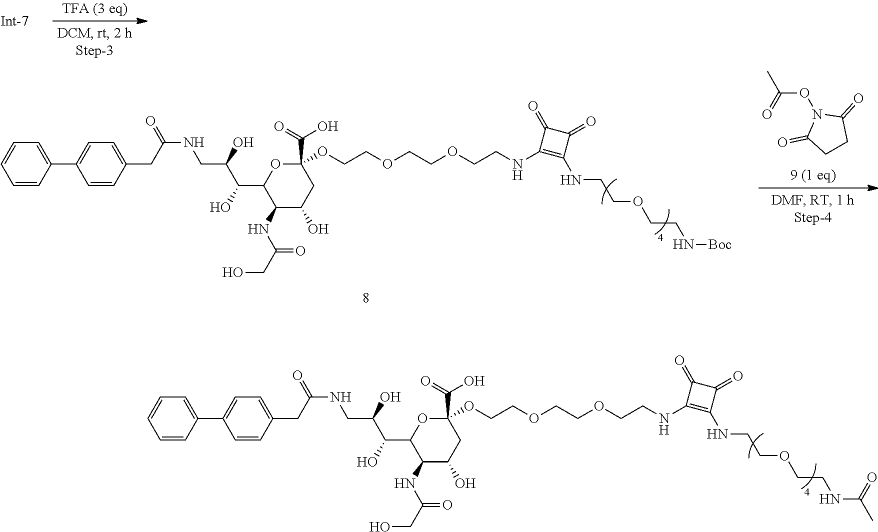

### Synthesis of 3-eth oxy-4-((2-(2-(2-hydroxyethoxy)ethoxy)ethyl)amino) cyclobut-3-ene-1,2-dione (3)

To a stirred solution of 2-(2-(2-aminoethoxy) ethoxy) ethan-1-ol (1) (4 g, 26.81 mmol) in Ethanol (40 mL) were added 3,4-diethoxycyclobut-3-ene-1,2-dione (6.83 g, 40.21 mmol) and triethyl amine (5.2 mL, 40.2 mmol) at room temperature. Then the resulting reaction mixture was stirred at 50° C. for 16 h. The reaction mixture gradually changed to a clear yellow solution. After completion, the reaction mixture was concentrated under reduced pressure and the crude residue was purified by Silica-gel column chromatography using 5% MeOH: DCM as eluents to afford the desired product 3-ethoxy-4-((2-(2-(2-hydroxyethoxy)ethoxy)ethyl)amino)cyclobut-3-ene-1,2-dione (3) as a yellow semi-solid, Yield: 2.9 g, 39%; LCMS (ESI+) m/z 274.3.1 [M+H]⁺.

### Synthesis of tert-butyl (14-((2-((2-(2-(2-hydroxyethoxy)ethoxy)ethyl)amino)-3,4-dioxocyclobut-1-en-1-yl)amino)-3,6,9,12-tetraoxatetradecyl) carbamate (5)

To a stirred solution of 3-ethoxy-4-((2-(2-(2-hydroxyethoxy)ethoxy)ethyl)amino) cyclobut-3-ene-1,2-dione (3) (2.9 g, 10.61 mmol) and tert-butyl (14-amino-3,6,9,12-tetraoxatetradecyl)carbamate (4) (3.57 g, 10.61 mmol) in Ethanol (30 mL) was added triethyl amine (2.2 mL, 15.91 mmol). The resulting reaction mixture was stirred at 50° C. for 16 h. After completion, the reaction mixture was concentrated under reduced pressure and the crude residue obtained was purified by Silica-gel column chromatography using 8% MeOH: DCM as eluents to afford the desired product tert-butyl (14-((2-((2-(2-(2-hydroxyethoxy)ethoxy)ethyl)amino)-3,4-dioxocyclobut-1-en-1-yl)amino)-3,6,9,12-tetraoxatetradecyl)carbamate (5) as a yellow solid, Yield: 2.5 g, 41%; LCMS (ESI+) m/z 564.7 [M+H]⁺.

### Synthesis of (R,2R)-3-(2-([1,1′-biphenyl]-4-yl)acetamido)-1-((2R,3R,4S,6R)-4-acetoxy-3-(2-acetoxyacetamido)-6-(2-(2-(2-((2-((2,2-dimethyl-4-oxo-3,8,11,14,17-pentaoxa-5-azanonadecan-19-yl)amino)-3,4-dioxocyclobut-1-en-1-yl)amino)ethoxy)ethoxy)ethoxy)-6-(methoxycarbonyl)tetrahydro-2H-pyran-2-yl)propane-1,2-diyl diacetate (6)

To a stirred solution of (R,2R)-3-(2-([1,1′-biphenyl]-4-yl)acetamido)-1-((2R,3R, 4S,6R)-4-acetoxy-3-(2-acetoxyacetamido)-6-(methoxycarbonyl)-6-(p-tolylthio)tetrahydro-2H-pyran-2-yl)propane-1,2-diyl diacetate (242_Int-6) (1 g, 1.24 mmol) and tert-butyl (14-((2-((2-(2-(2-hydroxyethoxy)ethoxy)ethyl)amino)-3,4-dioxocyclobut-1-en-1-yl)amino)-3,6,9,12-tetraoxatetradecyl)carbamate (5) (0.84 g, 1.48 mmol) in anhydrous dichloromethane (12 mL) and anhydrous acetonitrile (18 mL) were added Silver (1+) trifluoro methane sulfonate (AgOTf) (0.79 g, 3.1 mmol) and activated 4 Å powdered molecular sieves (1 g) and the reaction mixture was stirred at room temperature for 1 h under nitrogen atmosphere. Then the solution was cooled to −78° C., iodine monobromide (0.38 g, 1.86 mmol) dissolved in dichloromethane (2 mL) was added dropwise and the resulting mixture was stirred at the same temperature for 2 h. After completion, the reaction mixture was quenched by addition of triethylamine (pH˜7) and warmed to room temperature. The reaction mixture was filtered, the filtrate was washed with 10% aqueous sodium thiosulfate solution, dried over sodium sulfate, filtered, and concentrated under reduced pressure. The crude residue obtained was purified by prep-HPLC using acetonitrile and water (+0.05% TFA) as eluents to afford the desired product (1R,2R)-3-(2-([1,1′-biphenyl]-4-yl)acetamido)-1-((2R,3R,4S,6R)-4-acetoxy-3-(2-acetoxyacetamido)-6-(2-(2-(2-((2-((2,2-dimethyl-4-oxo-3,8,11,14,17-pentaoxa-5-azanonadecan-19-yl)amino)-3,4-dioxocyclobut-1-en-1-yl)amino)ethoxy)ethoxy)ethoxy)-6-(methoxycarbonyl)tetrahydro-2H-pyran-2-yl)propane-1,2-diyl diacetate (6) as a pale yellow solid, Yield: 0.56 g, 36%; LCMS (ESI+) m/z 1247.1 [M+H]⁺.

### Synthesis of (2R,4S,5R,6R)-6-((1R,2R)-3-(2-([1,1′-biphenyl]-4-yl)acetamido)-1,2-dihydroxypropyl)-2-(2-(2-(2-((2-((2,2-dimethyl-4-oxo-3,8,11,14,17-pentaoxa-5-azanonadecan-19-yl)amino)-3,4-dioxocyclobut-1-en-1-yl)amino)ethoxy)ethoxy)ethoxy)-4-hydroxy-5-(2-hydroxyacetamido)tetrahydro-2H-pyran-2-carboxylic acid (7)

To a stirred solution (1R,2R)-3-(2-([1,1′-biphenyl]-4-yl)acetamido)-1-((2R,3R,4S,6R)-4-acetoxy-3-(2-acetoxyacetamido)-6-(2-(2-(2-((2-((2,2-dimethyl-4-oxo-3,8,11,14,17-pentaoxa-5-azanonadecan-19-yl)amino)-3,4-dioxocyclobut-1-en-1-yl)amino)ethoxy)ethoxy)ethoxy)-6-(methoxycarbonyl)tetrahydro-2H-pyran-2-yl)propane-1,2-diyl diacetate (6) (0.56 g, 0.44 mmol) in tetrahydrofuran (4.2 mL) was added a solution of lithium hydroxide monohydrate (110 mg, 2.69 mmol) dissolved in water (1.8 mL). The reaction mixture was stirred at room temperature for 6 h. After completion, the reaction mixture was acidified with Dowex (H⁺ exchange resin) to maintain pH˜6 and the suspension was filtered, concentrated under reduced pressure to get the crude material, which was purified by prep-HPLC using acetonitrile and water (+0.05% TFA) as eluents, to get (2R,4S,5R,6R)-6-((1R,2R)-3-(2-([1,1′-biphenyl]-4-yl)acetamido)-1,2-dihydroxypropyl)-2-(2-(2-(2-((2-((2,2-dimethyl-4-oxo-3,8,11,14,17-pentaoxa-5-azanonadecan-19-yl)amino)-3,4-dioxocyclobut-1-en-1-yl)amino)ethoxy)ethoxy)ethoxy)-4-hydroxy-5-(2-hydroxyacetamido)tetrahydro-2H-pyran-2-carboxylic acid (7) as off white solid (Yield-190 mg, 39%). LCMS (ESI+) m/z 1065.1 [M+H]⁺.

### Synthesis of (2R,4S,5R,6R)-6-((1R,2R)-3-(2-([1,1′-biphenyl]-4-yl)acetamido)-1,2-dihydro xypropyl)-2-(2-(2-(2-((2-((14-amino-3,6,9,12-tetraoxatetradecyl)amino)-3,4-dioxocyclobut-1-en-1-yl)amino)ethoxy)ethoxy)ethoxy)-4-hydroxy-5-(2-hydroxyacetamido)tetrahydro-2H-pyran-2-carboxylic acid (8)

To a stirred solution of (2R,4S,5R,6R)-6-((1R,2R)-3-(2-([1,1′-biphenyl]-4-yl)acetamido)-1,2-dihydroxypropyl)-2-(2-(2-(2-((2-((2,2-dimethyl-4-oxo-3,8,11,14,17-pentaoxa-5-azanonadecan-19-yl)amino)-3,4-dioxocyclobut-1-en-1-yl)amino)ethoxy)ethoxy)ethoxy)-4-hydroxy-5-(2-hydroxyacetamido)tetrahydro-2H-pyran-2-carboxylic acid (7) (190 mg, 0.17 mmol) in dichloromethane (1.9 mL) was added trifluoro acetic acid (0.9 mL) dropwise at 0° C. and the resulting reaction mixture was stirred at room temperature for 3 h. After completion, the solvent was concentrated to get crude which was suspended in methanol and neutralized with Tetra alkyl ammonium carbonate, the polymer-bound resin. Resulting mixture was filtered and filtrate was concentrated to get (2R,4S,5R,6R)-6-((1R,2R)-3-(2-([1,1′-biphenyl]-4-yl)acetamido)-1,2-dihydroxypropyl)-2-(2-(2-(2-((2-((14-amino-3,6,9,12-tetraoxatetradecyl)amino)-3,4-dioxocyclobut-1-en-1-yl)amino)ethoxy)ethoxy)ethoxy)-4-hydroxy-5-(2-hydroxyacetamido)tetrahydro-2H-pyran-2-carboxylic acid (8) as yellow solid, Yield: 120 mg, 69%; LCMS (ESI+) m/z 965 [M+H]⁺.

### Synthesis of (2R,4S,5R,6R)-6-((1R,2R)-3-(2-([1,1′-biphenyl]-4-yl)acetamido)-1,2-dihydroxypropyl)-2-(2-(2-(2-((3,4-dioxo-2-((2-oxo-6,9,12,15-tetraoxa-3-azaheptadecan-17-yl)amino)cyclobut-1-en-1-yl)amino)ethoxy)ethoxy)ethoxy)-4-hydroxy-5-(2-hydroxy acetamido)tetrahydro-2H-pyran-2-carboxylic acid (242)

To a stirred solution of (2R,4S,5R,6R)-6-((1R,2R)-3-(2-([1,1′-biphenyl]-4-yl)acetamido)-1,2-dihydroxypropyl)-2-(2-(2-(2-((2-((14-amino-3,6,9,12-tetraoxatetradecyl)amino)-3,4-dioxocyclobut-1-en-1-yl)amino)ethoxy)ethoxy)ethoxy)-4-hydroxy-5-(2-hydroxyacetamido) tetrahydro-2H-pyran-2-carboxylic acid (8) (110 mg, 0.11 mmol) in DMF (1.1 mL) was added 2,5-dioxopyrrolidin-1-yl acetate (9) (17.9 mg, 0.11 mmol) at 0° C. The resulting reaction mixture was stirred at 0° C. for 1 h. After completion, the reaction mixture was purified by prep-HPLC using acetonitrile and water (+0.05% TFA) as eluents to afford the desired product (2R,4S,5R,6R)-6-((1R,2R)-3-(2-([1,1′-biphenyl]-4-yl)acetamido)-1,2-dihydroxypropyl)-2-(2-(2-(2-((3,4-dioxo-2-((2-oxo-6,9,12,15-tetraoxa-3-azaheptadecan-17-yl)amino)cyclobut-1-en-1-yl)amino)ethoxy)ethoxy)ethoxy)-4-hydroxy-5-(2-hydroxyacetamido)tetrahydro-2H-pyran-2-carboxylic acid (242) as white solid, Yield: 10 mg, 8.7%; LCMS (ESI+) m/z 1006.0 [M+H]⁺. ¹H NMR (400 MHz, MeOD) δ 7.63-7.58 (m, 4H), 7.45-7.33 (m, 5H), 4.04 (s, 2H), 3.92-3.72 (m, 10H), 3.71-3.61 (m, 23H), 3.55-3.52 (m, 2H), 3.40-3.32 (m, 6H), 2.40 (dd, J=12.3, 4.0 Hz, 1H), 1.95 (s, 3H), 1.73 (t, J=11.9 Hz, 1H), 1.30 (s, 1H).

### (2R,4S,5R,6R)-6-((1R,2R)-3-(2-([1,1′-biphenyl]-4-yl)acetamido)-1,2-dihydroxypropyl)-2-(2-(2-(2-((2-((18-(2,5-dioxo-2,5-dihydro-1H-pyrrol-1-yl)-16-oxo-3,6,9,12-tetraoxa-15-azaoctadecyl)amino)-3,4-dioxocyclobut-1-en-1-yl)amino)ethoxy)ethoxy)ethoxy)-4-hydroxy-5-(2-hydroxyacetamido)tetrahydro-2H-pyran-2-carboxylic acid (Cpd. 242M)

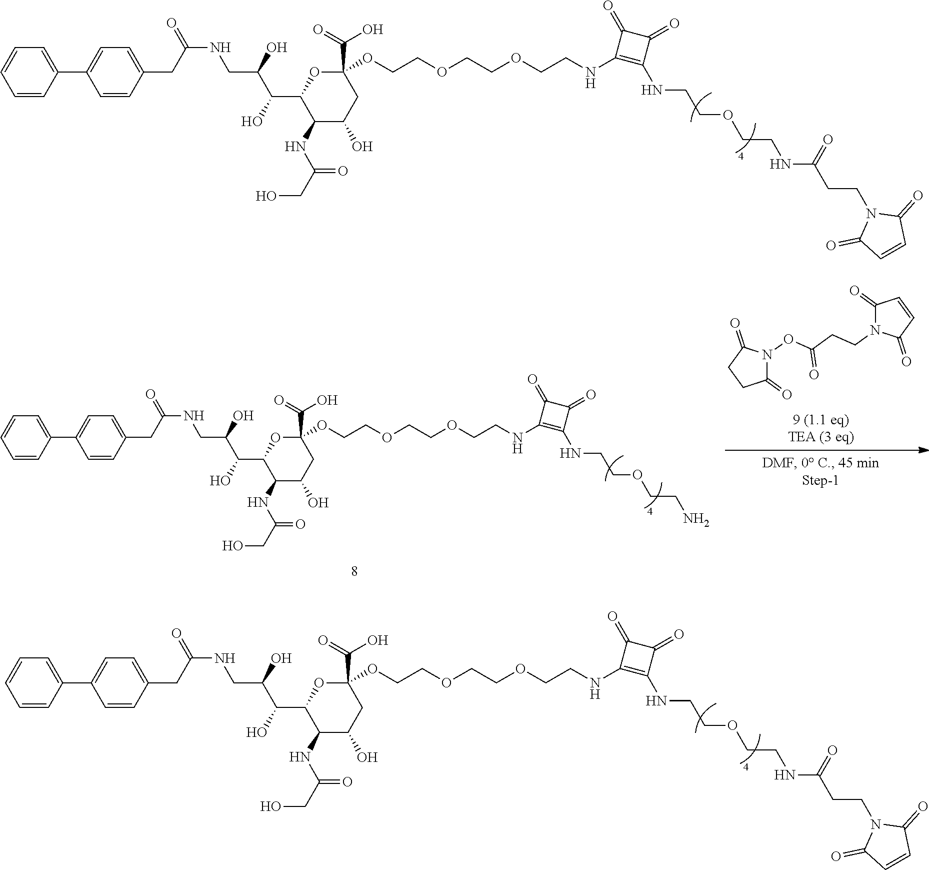

### Synthesis of (2R,4S,5R,6R)-6-((1R,2R)-3-(2-([1,1′-biphenyl]-4-yl)acetamido)-1,2-dihydroxypropyl)-2-(2-(2-(2-(4-(17-(2,5-dioxo-2,5-dihydro-1H-pyrrol-1-yl)-15-oxo-2,5,8,11-tetraoxa-14-azaheptadecyl)-1H-1,2,3-triazol--yl)ethoxy)ethoxy)ethoxy)-4-hydroxy-5-(2-hydroxyacetamido)tetrahydro-2H-pyran-2-carboxylic acid (242M)

To a stirred solution of (2R,4S,5R,6R)-6-((1R,2R)-3-(2-([1,1′-biphenyl]-4-yl)acetamido)-1,2-dihydroxypropyl)-2-(2-(2-(2-((2-((14-amino-3,6,9,12-tetraoxatetradecyl)amino)-3,4-dioxocyclobut-1-en-1-yl)amino)ethoxy)ethoxy)ethoxy)-4-hydroxy-5-(2-hydroxyacetamido) tetrahydro-2H-pyran-2-carboxylic acid (8) (120 mg, 0.12 mmol) in DMF (2 mL) was added 2,5-dioxopyrrolidin-1-yl 3-(2,5-dioxo-2,5-dihydro-1H-pyrrol-1-yl)propanoate (9) (36.3 mg, 0.136 mmol) at 0° C., followed by addition of triethylamine (0.05 mL, 0.37 mmol). The resulting reaction mixture was stirred at 0° C. for 1 h. After completion, the reaction mixture was purified by prep-HPLC purification to afford the desired material (2R,4S,5R,6R)-6-((1R,2R)-3-(2-([1,1′-biphenyl]-4-yl)acetamido)-1,2-dihydroxypropyl)-2-(2-(2-(2-(4-(17-(2,5-dioxo-2,5-dihydro-1H-pyrrol-1-yl)-15-oxo-2,5,8,11-tetraoxa-14-azahepta decyl)-1H-1,2,3-triazol--yl)ethoxy)ethoxy)ethoxy)-4-hydroxy-5-(2-hydroxyacetamido) tetrahydro-2H-pyran-2-carboxylic acid (242M) as white solid, Yield: 30 mg, 20%; LCMS (ESI+) m/z 1115.9 [M+H]⁺. ¹H NMR (400 MHz, MeOD) δ 7.63-7.58 (m, 4H), 7.45-7.39 (m, 4H), 7.35-7.31 (m, 1H), 6.83 (s, 2H), 4.04 (s, 2H), 3.93-3.74 (m, 12H), 3.71-3.58 (m, 26H), 3.52-3.22 (m, 5H), 2.73-2.72 (m, 1H), 2.48 (t, J=6.8 Hz, 1H), 1.75 (t, J=11.9 Hz, 1H).

### (2R,4S,5R,6R)-6-((1R,2R)-3-(2-([1,1′-biphenyl]-4-yl) acetamido)-1,2-dihydroxypropyl)-4-hydroxy-5-(2-hydroxyacetamido)-2-(2-(2-(2-(4-(15-oxo-2,5,8,11-tetraoxa-14-azahexadecyl)-1H-1,2,3-triazol-1-yl) ethoxy) ethoxy) ethoxy) tetrahydro-2H-pyran-2-carboxylic acid (Cpd. 243)

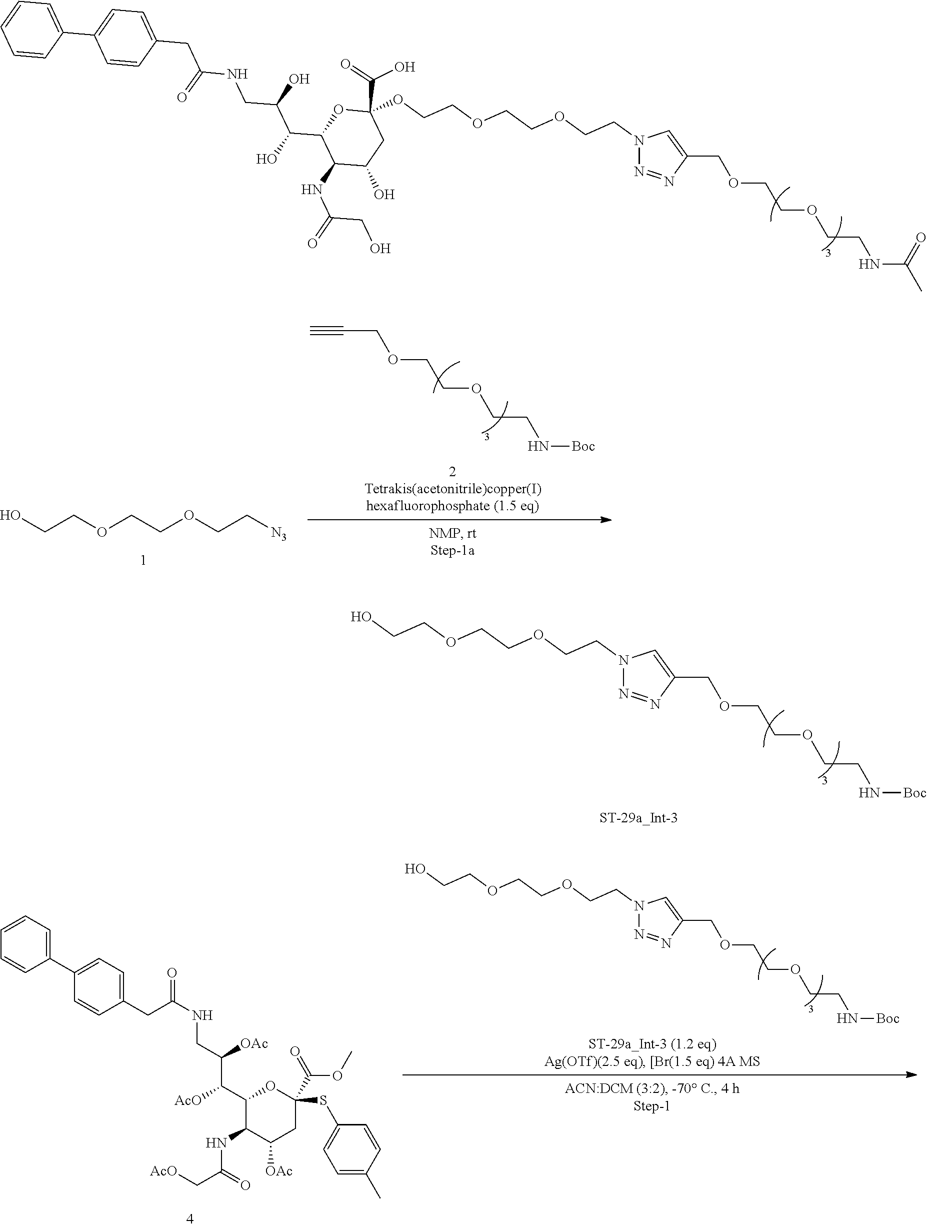

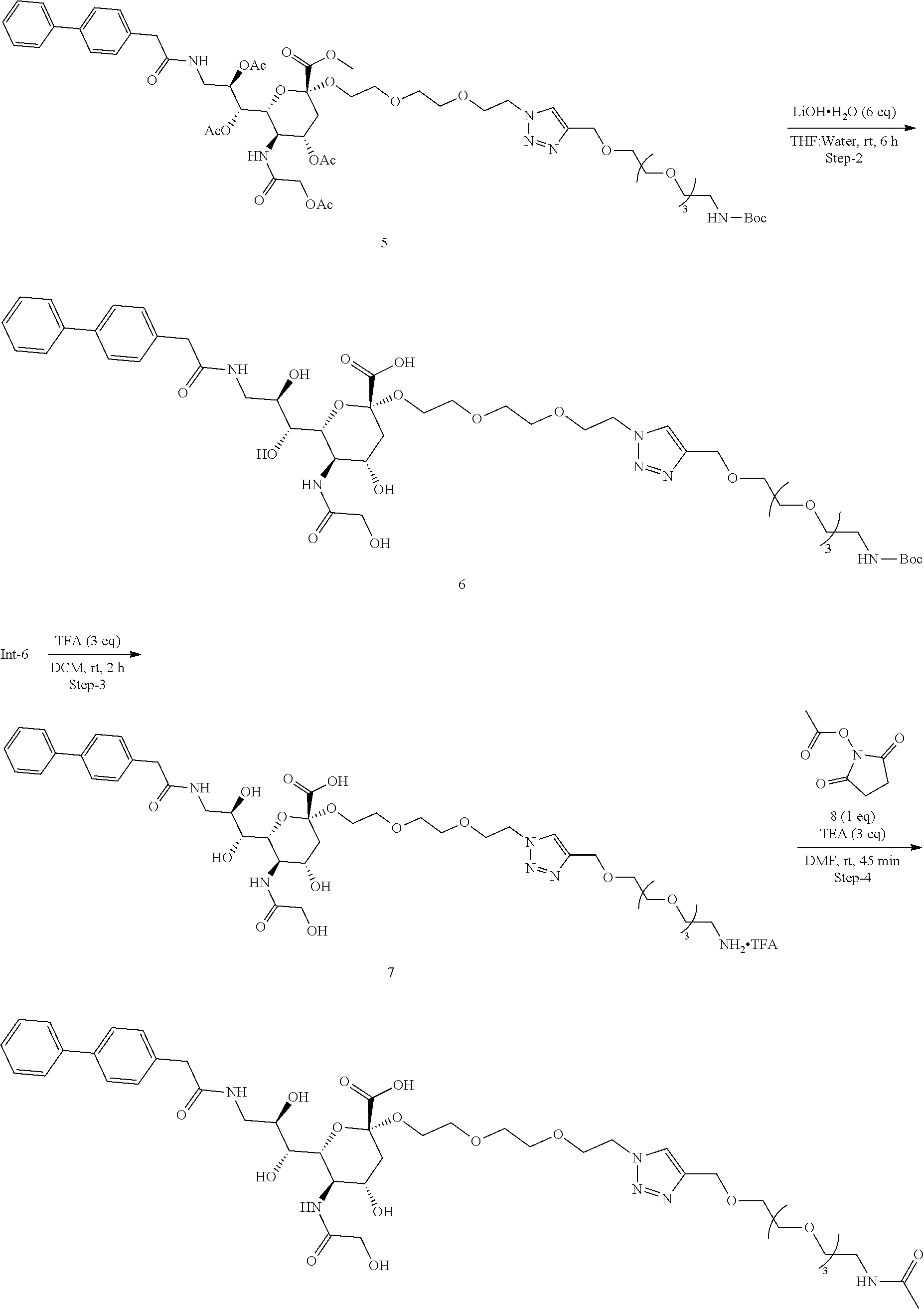

### Synthesis of tert-butyl (1-(1-(2-(2-(2-hydroxyethoxy)ethoxy)ethyl)-1H-1,2,3-triazol-4-yl)-2,5,8,11-tetraoxatridecan-13-yl) carbamate (243_Int-3)

To a stirred solution of 2-(2-(2-azidoethoxy) ethoxy) ethan-1-ol (1) (1 g, 5.7 mmol) and added tert-butyl (3,6,9,12-tetraoxapentadec-14-yn-1-yl) carbamate (2) (2.31 g, 5.70 mmol) in anhydrous N-methyl pyrrolidone (10 mL) was added Tetrakis(acetonitrile)copper(l) hexa-fluoro phosphate (3.19 g, 5.8 mmol) under nitrogen atmosphere. The reaction mixture was stirred at room temperature for 2 h. After completion, the mixture was filtered, few drops of AcOH were added and the filtrate was purified by C18 reverse phase column chromatography using water and acetonitrile (+0.05% TFA) as eluents to afford tert-butyl (1-(1-(2-(2-(2-hydroxyethoxy)ethoxy)ethyl)-1H-1,2,3-triazol-4-yl)-2,5,8,11-tetraoxatridecan-13-yl)carbamate (243_Int-3). Yield: 1.8 g, 62%. ¹H NMR (400 MHz DMSO-d6): δ 8.07 (s, 1H), 6.75 (s, 1H), 4.52-4.50 (m, 4H), 3.81 (t, J=5.2 Hz, 2H), 3.55-3.53 (m, 10H), 3.54-3.43 (m, 7H), 3.93-3.35 (m, 6H), 3.08-3.03 (m, 2H), 1.37 (s, 9H).

### Synthesis of (1R,2R)-3-(2-([1,1′-biphenyl]-4-yl) acetamido)-1-((2R,3R,4S,6R)-4-acetoxy-3-(2-acetoxyacetamido)-6-(2-(2-(2-(4-(17,17-dimethyl-15-oxo-2,5,8,11,16-pentaoxa-14-azaoctadecyl)-1H-1,2,3-triazol-1-yl) ethoxy) ethoxy) ethoxy)-6-(methoxycarbonyl) tetrahydro-2H-pyran-2-yl) propane-1,2-diyl diacetate (5)

To a stirred solution of (1R,2R)-3-(2-([1,1′-biphenyl]-4-yl)acetamido)-1-((2R,3R,4S,6R)-4-acetoxy-3-(2-acetoxyacetamido)-6-(methoxycarbonyl)-6-(p-tolylthio)tetrahydro-2H-pyran-2-yl)propane-1,2-diyl diacetate (4) (1 g, 1.24 mmol) and tert-butyl (1-(1-(2-(2-(2-hydroxyethoxy)ethoxy)ethyl)-1H-1,2,3-triazol-4-yl)-2,5,8,11-tetraoxatridecan-13-yl)carbamate (0.75 g, 1.48 mmol) in anhydrous acetonitrile (15 mL) and anhydrous dichloromethane (10 mL) were added powdered molecular sieves 4 Å (2 g) and Ag(OTf) (0.79 g, 3.1 mmol) under argon atmosphere. The reaction mixture was stirred for 30 min., then cooled to −78° C. A solution of iodine monobromide (0.38 g, 1.86 mmol) dissolved in dichloromethane (2 ml) was added. The reaction mixture was stirred at the same temperature for 2 h. After completion, the reaction mixture was diluted with dichloromethane (15 ml), quenched with triethylamine (up to pH=7) and filtered, filtrate was washed with 10% aqueous sodium thio-sulfate solution, dried over sodium sulfate, concentrated. The crude residue obtained was purified by C18 reverse phase column chromatography using acetonitrile and water (+0.05%, TFA) as eluents to afford (1R,2R)-3-(2-([1,1′-biphenyl]-4-yl) acetamido)-1-((2R,3R,4S,6R)-4-acetoxy-3-(2-acetoxyacetamido)-6-(2-(2-(2-(4-(17,17-dimethyl-15-oxo-2,5,8,11,16-pentaoxa-14-azaoctadecyl)-1H-1,2,3-triazol-1-yl) ethoxy) ethoxy) ethoxy)-6-(methoxycarbonyl) tetrahydro-2H-pyran-2-yl) propane-1,2-diyl diacetate (5) as yellow solid. Yield: 900 mg, 61%; LCMS (ESI±) m/z 1190.2 [M+H]⁺.

### Synthesis of (2R,4S,5R,6R)-6-((1R,2R)-3-(2-([1,1′-biphenyl]-4-yl) acetamido)-1,2-dihydroxypropyl)-2-(2-(2-(2-(4-(17,17-dimethyl-15-oxo-2,5,8,11,16-pentaoxa-14-azaoctadecyl)-1H-1,2,3-triazol-1-yl) ethoxy) ethoxy) ethoxy)-4-hydroxy-5-(2-hydroxyacetamido) tetrahydro-2H-pyran-2-carboxylic acid (6)

To a stirred solution of (1R,2R)-3-(2-([1,1′-biphenyl]-4-yl) acetamido)-1-((2R,3R,4S,6R)-4-acetoxy-3-(2-acetoxyacetamido)-6-(2-(2-(2-(4-(17,17-dimethyl-15-oxo-2,5,8,11,16-pentaoxa-14-azaoctadecyl)-1H-1,2,3-triazol-1-yl) ethoxy) ethoxy) ethoxy)-6-(methoxycarbonyl) tetrahydro-2H-pyran-2-yl) propane-1,2-diyl diacetate (5) (0.9 g, 0.89 mmol) in tetrahydrofuran (9 mL) was added aq. solution of lithium hydroxide monohydrate (190 mg, 4.53 mmol) at 0° C. and the reaction mixture was stirred at room temperature for 16 h. After completion the reaction mixture was treated with acidic resin (Dowex H⁺) to pH˜6 and the suspension was filtered. The filtrate was concentrated and the crude residue obtained was purified by C18 reverse phase column chromatography using acetonitrile and water (0.05%, TFA) as eluents to afford (2R,4S,5R,6R)-6-((1R,2R)-3-(2-([1,1′-biphenyl]-4-yl) acetamido)-1,2-dihydroxypropyl)-2-(2-(2-(2-(4-(17,17-dimethyl-15-oxo-2,5,8,11,16-pentaoxa-14-azaoctadecyl)-1H-1,2,3-triazol-1-yl) ethoxy) ethoxy) ethoxy)-4-hydroxy-5-(2-hydroxyacetamido) tetrahydro-2H-pyran-2-carboxylic acid (6) as off white solid. Yield:400 mg, 52%. LCMS (ESI±) m/z 1008 [M−H]⁻.

### Synthesis of (2R,4S,5R,6R)-6-((1R,2R)-3-(2-([1,1′-biphenyl]-4-yl) acetamido)-1,2-dihydroxy propyl)-2-(2-(2-(2-(4-(13-amino-2,5,8,11-tetraoxatridecyl)-1H-1,2,3-triazol-1-yl) ethoxy) ethoxy) ethoxy)-4-hydroxy-5-(2-hydroxyacetamido) tetrahydro-2H-pyran-2-carboxylic acid (7)

To a stirred solution of (2R,4S,5R,6R)-6-((1R,2R)-3-(2-([1,1′-biphenyl]-4-yl) acetamido)-1,2-dihydroxypropyl)-2-(2-(2-(2-(4-(17,17-dimethyl-15-oxo-2,5,8,11,16-pentaoxa-14-azaoctadecyl)-1H-1,2,3-triazol-1-yl) ethoxy) ethoxy) ethoxy)-4-hydroxy-5-(2-hydroxyacetamido) tetrahydro-2H-pyran-2-carboxylic acid (7) (400 mg, 0.44 mmol) in anhydrous dichloromethane (6 mL) was added trifluoro acetic acid (0.4 mL) at 0° C. and the reaction mixture was stirred at room temperature for 2 h. After completion, the mixture was concentrated, and the crude residue obtained was suspended in methanol and neutralized with tetra alkyl ammonium carbonate (polymer-supported). The mixture was filtered and the filtrate was concentrated to afford (2R,4S,5R,6R)-6-((1R,2R)-3-(2-([1,1′-biphenyl]-4-yl) acetamido)-1,2-dihydroxy propyl)-2-(2-(2-(2-(4-(13-amino-2,5,8,11-tetraoxatridecyl)-1H-1,2,3-triazol-1-yl) ethoxy) ethoxy) ethoxy)-4-hydroxy-5-(2-hydroxyacetamido) tetrahydro-2H-pyran-2-carboxylic acid (7) as yellow solid (Yield:250 mg, 69%); LCMS (ESI±) m/z 908.0 [M+H]⁺.

### Synthesis of (2R,4S,5R,6R)-6-((1R,2R)-3-(2-([1,1′-biphenyl]-4-yl) acetamido)-1,2-dihydroxypropyl)-4-hydroxy-5-(2-hydroxyacetamido)-2-(2-(2-(2-(4-(15-oxo-2,5,8,11-tetraoxa-14-azahexadecyl)-1H-1,2,3-triazol-1-yl) ethoxy) ethoxy) ethoxy) tetrahydro-2H-pyran-2-carboxylic acid (243)

To a stirred solution of (2R,4S,5R,6R)-6-((1R,2R)-3-(2-([1,1′-biphenyl]-4-yl)acetamido)-1,2-dihydroxy propyl)-2-(2-(2-(2-(4-(13-amino-2,5,8,11-tetraoxatridecyl)-1H-1,2,3-triazol-1-yl)ethoxy) ethoxy)ethoxy)-4-hydroxy-5-(2-hydroxyacetamido)tetrahydro-2H-pyran-2-carboxylic acid (7) (60 mg, 0.07 mmol) in N,N-Dimethyl formamide (0.5 mL) were added 2,5-dioxopyrrolidin-1-yl acetate (8) (10.39 mg, 0.06 mmol) and triethylamine at 0° C. The resulting reaction mixture was stirred at room temperature for 45 min. After completion, the reaction mixture was concentrated and purified by C18 prep-HPLC, using acetonitrile and water (+0.05%, TFA) as eluents to afford (2R,4S,5R,6R)-6-((1R,2R)-3-(2-([1,1′-biphenyl]-4-yl) acetamido)-1,2-dihydroxypropyl)-4-hydroxy-5-(2-hydroxyacetamido)-2-(2-(2-(2-(4-(15-oxo-2,5,8,11-tetraoxa-14-azahexadecyl)-1H-1,2,3-triazol-1-yl) ethoxy) ethoxy) ethoxy) tetrahydro-2H-pyran-2-carboxylic acid (243) as white solid. Yield: 11 mg, 17%; LCMS (ESI±) m/z 949.0 [M+H]⁺. ¹H NMR (400 MHz, MeOD): δ 8.03 (s, 1H), 7.79 (s, 1H), (7.59 (dd, J=11.7, 7.8 Hz, 4H), 7.45-7.36 (m, 4H), 7.33 (d, J=7.8 Hz, 1H), 4.64 (s, 2H), 4.57 (t, J=5.2 Hz, 2H), 4.03 (s, 2H), 3.86 (dd, J=13.0, 7.9 Hz, 8H), 3.67-3.53 (m, 22H), 3.40-3.36 (m, 4H), 3.27-3.18 (m, 1H), 2.73 (d, J=12.5 Hz, 1H), 1.94 (s, 3H), 1.74 (s, 1H).

### (2R,4S,5R,6R)-6-((1R,2R)-3-(2-([1,1′-biphenyl]-4-yl) acetamido)-1,2-dihydroxypropyl)-2-(2-(2-(2-(4-(17-(2,5-dioxo-2,5-dihydro-1H-pyrrol-1-yl)-15-oxo-2,5,8,11-tetraoxa-14-azaheptadecyl)-1H-1,2,3-triazol-1-yl) ethoxy) ethoxy) ethoxy)-4-hydroxy-5-(2-hydroxyacetamido) tetrahydro-2H-pyran-2-carboxylic acid (Cpd. 243M)

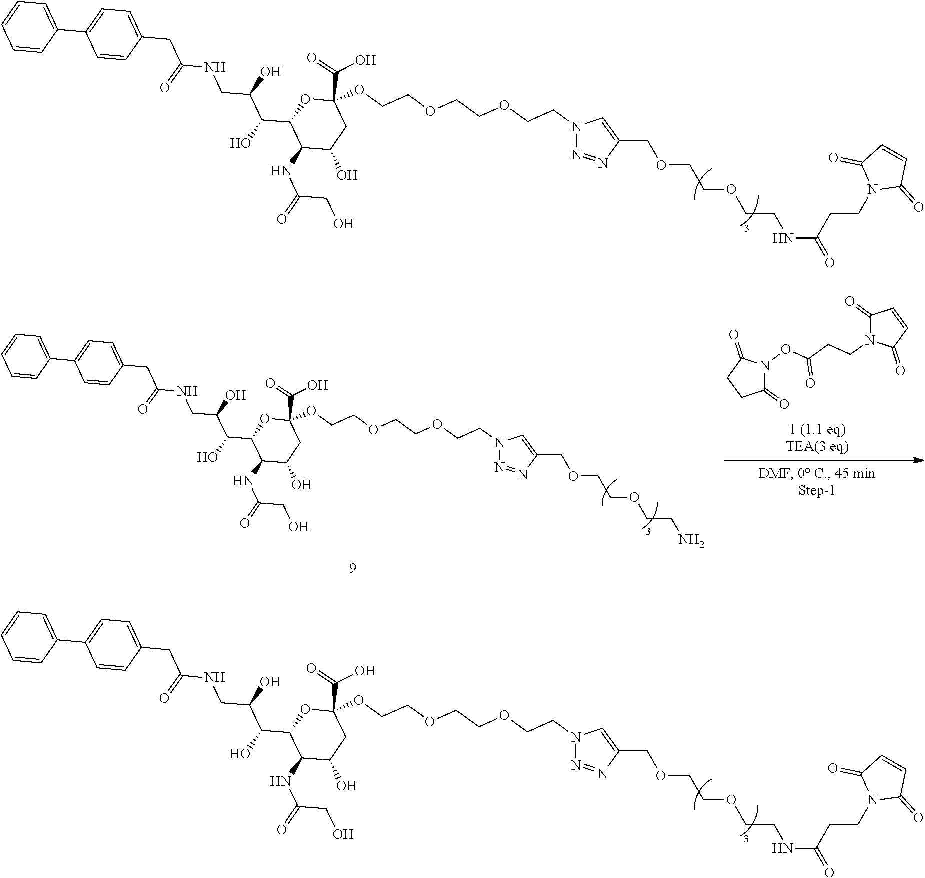

### Synthesis of (2R,4S,5R,6R)-6-((1R,2R)-3-(2-([1,1′-biphenyl]-4-yl) acetamido)-1,2-dihydroxypropyl)-2-(2-(2-(2-(4-(17-(2,5-dioxo-2,5-dihydro-1H-pyrrol-1-yl)-15-oxo-2,5,8,11-tetraoxa-14-azaheptadecyl)-1H-1,2,3-triazol-1-yl) ethoxy) ethoxy) ethoxy)-4-hydroxy-5-(2-hydroxyacetamido) tetrahydro-2H-pyran-2-carboxylic acid (243M)

To a stirred solution of (2R,4S,5R,6R)-6-((1R,2R)-3-(2-([1,1′-biphenyl]-4-yl)acetamido)-1,2-dihydroxypropyl)-2-(2-(2-(2-(4-(13-amino-2,5,8,11-tetraoxatridecyl)-1H-1,2,3-triazol-1-yl)ethoxy)ethoxy)ethoxy)-4-hydroxy-5-(2-hydroxyacetamido)tetrahydro-2H-pyran-2-carboxylic acid (7) (190 mg, 0.21 mmol) and 2,5-dioxopyrrolidin-1-yl 3-(2,5-dioxo-2,5-dihydro-1H-pyrrol-1-yl)propanoate (1) (55.7 mg, 0.21 mmol) in N,N-Dimethyl formamide (2 mL) was added triethylamine (0.09 mL, 0.62 mmol) at 0° C. and the resulting reaction mixture was stirred at 0° C. for 45 min. After completion, the reaction mixture was purified by C18 prep-HPLC by using acetonitrile and water (0.05%, TFA) as eluents to afford (2R,4S,5R,6R)-6-((1R,2R)-3-(2-([1,1′-biphenyl]-4-yl)acetamido)-1,2-dihydroxypropyl)-2-(2-(2-(2-(4-(17-(2,5-dioxo-2,5-dihydro-1H-pyrrol-1-yl)-15-oxo-2,5,8,11-tetraoxa-14-azahepta decyl)-1H-1,2,3-triazol-1-yl)ethoxy)ethoxy)ethoxy)-4-hydroxy-5-(2-hydroxyacetamido) tetrahydro-2H-pyran-2-carboxylic acid (243M) as white solid. Yield: 34 mg, 15%; LCMS (ESI±) m/z 1058.0 [M+H]⁺. ¹H NMR (400 MHz, MeOD-d4): δ 8.03 (d, J=5.2 Hz, 1H), 7.88 ((d, J=7.6 Hz, 1H), 7.60 (dd, J=11.2, 7.7 Hz, 4H), 7.48-7.37 (m, 4H), 7.33 (t, J=7.3 Hz, 1H), 6.82 (s, 2H), 4.64 (s, 2H), 4.58 (t, J=5.0 Hz, 2H), 4.03 (s, 2H), 4.00-3.78 (m, 7H), 3.83-3.69 (m, 5H), 3.67 (q, J=2.0 Hz, 4H), 3.61-3.64 (m, 6H), 3.61-3.53 (m, 10H), 3.50 (t, J=5.3 Hz, 2H), 3.43-3.38 (m, 2H), 3.30 (d, J=5.5 Hz, 2H), 3.27-3.22 (m, 1H), 2.81-2.65 (m, 1H), 2.47 (t, J=7.0 Hz, 2H), 1.75 (t, J=11.8 Hz, 1H).

### (2R,4S,5R,6R)-6-((1R,2R)-3-(2-(4-chlorophenyl) acetamido)-1,2-dihydroxypropyl)-2-((2,18-dioxo-6,9,12,15-tetraoxa-3,19-diazapentacosan-25-yl) oxy)-4-hydroxy-5-(2-hydroxyacetamido) tetrahydro-2H-pyran-2-carboxylic acid (Cpd. 244)

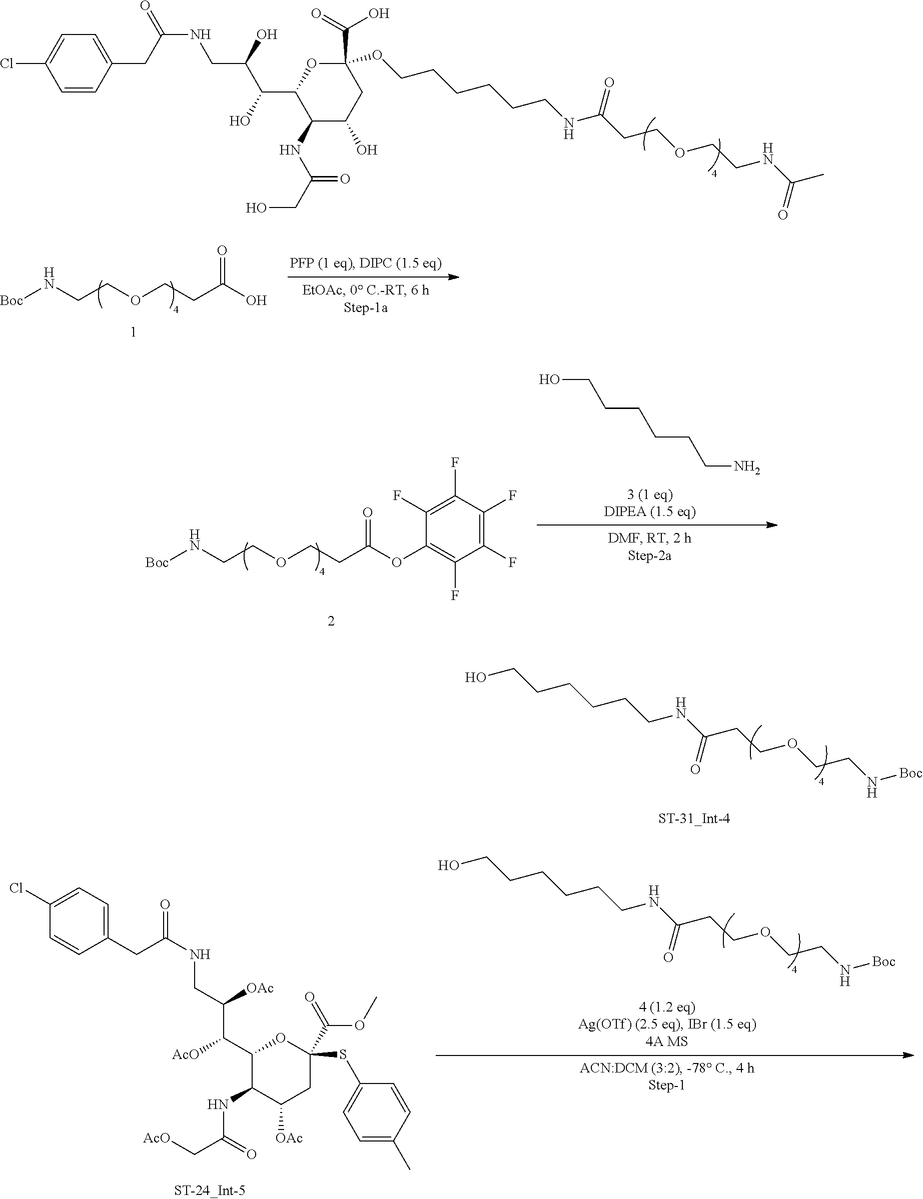

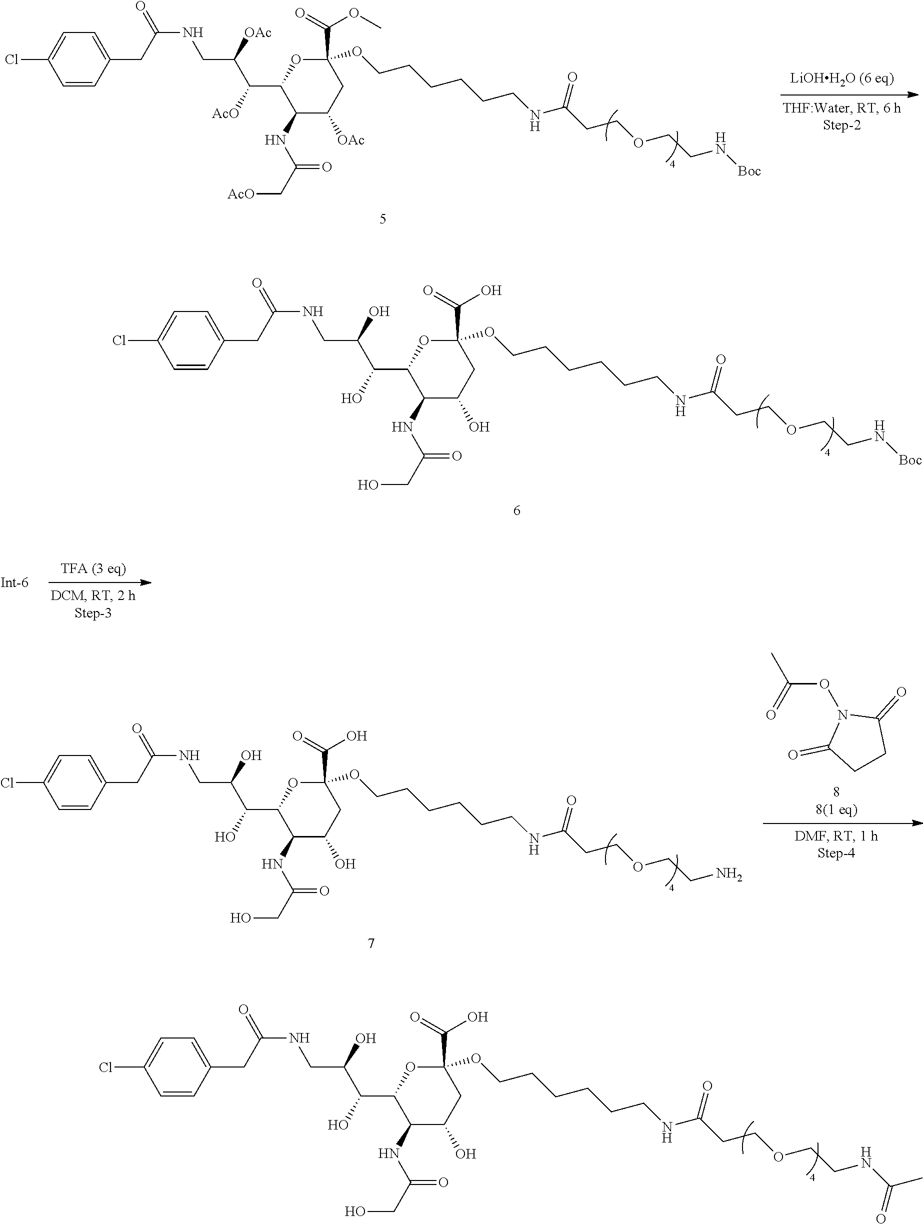

### Synthesis of perfluorophenyl 2,2-dimethyl-4-oxo-3,8,11,14,17-pentaoxa-5-azaicosan-20-oate (2)

To a stirred solution of 2,2-dimethyl-4-oxo-3,8,11,14,17-pentaoxa-5-azaicosan-20-oic acid (1) (5 g, 13.6 mmol) and penta-fluoro phenol (2.5 g, 13.6 mmol) in ethyl acetate (50 mL) was added di-iso-propyl carbodimide (2.58 g, 20.4 mmol) dropwise at 0° C. The reaction mixture was stirred at room temperature for 6 h. After completion, the reaction mixture was filtered, filtrate was concentrated, the crude residue was purified by silica gel column chromatography using ethyl acetate and hexane as eluents to afford perfluorophenyl 2,2-dimethyl-4-oxo-3,8,11,14,17-pentaoxa-5-azaicosan-20-oate (2) as off-white solid. Yield: 5 g, 69%; LCMS (ESI±) m/z 533.65 [M+H]⁺.

### Synthesis of tert-butyl (22-hydroxy-15-oxo-3,6,9,12-tetraoxa-16-azadocosyl) carbamate (ST-31_Int-4)

To a stirred solution of perfluorophenyl 2,2-dimethyl-4-oxo-3,8,11,14,17-pentaoxa-5-azaicosan-20-oate (2) (4 g, 7.52 mmol) and 6-aminohexan-1-ol (3) (0.88 g, 7.52 mmol) in N,N-dimethylformamide (10 mL) was added di-isopropyl ethylamine (2.08 ml, 11.29 mmol) at 0° C. The resulting reaction mixture was stirred at room temperature for 2 h. After completion, the reaction mixture was concentrated and purified by silica gel column chromatography using ethyl acetate and hexane as eluents to afford tert-butyl (22-hydroxy-15-oxo-3,6,9,12-tetraoxa-16-azadocosyl) carbamate (ST-31_Int-4) as white solid. Yield: 2.4 g, 71%; LCMS (ESI±) m/z 465.5 [M+H]⁺.

### Synthesis of (1R,2R)-1-((2R,3R,4S,6R)-4-acetoxy-3-(2-acetoxyacetamido)-6-((2,2-dimethyl-4,20-dioxo-3,8,11,14,17-pentaoxa-5,21-diazaheptacosan-27-yl) oxy)-6-(methoxycarbonyl) tetrahydro-2H-pyran-2-yl)-3-(2-(4-chlorophenyl) acetamido) propane-1,2-diyl diacetate (5)

To a stirred solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (ST-24_Int-5) (1 g, 1.38 mmol) and tert-butyl (22-hydroxy-15-oxo-3,6,9,12-tetraoxa-16-azadocosyl)carbamate (ST-31_Int-4) (0.91 g, 1.96 mmol) in anhydrous dichloromethane (12 mL) and anhydrous acetonitrile (24 mL) mixed solvent, were added Silver (+1) trifluoro methane sulfonate [Ag(OTf)] (0.83 g, 3.27 mmol) and activated 4 Å powdered molecular sieves (5 g). The reaction mixture was stirred at room temperature for 1 h under nitrogen atmosphere. Next the reaction mixture was cooled to −78° C. and a solution of iodine monobromide (0.4 g, 1.96 mmol, 1.5 eq.) dissolved in dichloromethane (4 mL) was added dropwise. The reaction mixture was stirred at the same temperature for 2 h. After completion, the reaction mixture was quenched with triethylamine (5 mL) and warmed to room temperature. The reaction mixture was filtered, filtrate was washed by saturated aqueous sodium thiosulfate solution and dried over sodium sulfate, filtered and concentrated. The crude residue obtained was purified by C18 reverse phase column chromatography using acetonitrile and water (+0.05%, TFA) as eluents to afford, (1R,2R)-1-((2R,3R,4S,6R)-4-acetoxy-3-(2-acetoxyacetamido)-6-((2,2-dimethyl-4,20-dioxo-3,8,11,14,17-pentaoxa-5,21-diazaheptacosan-27-yl) oxy)-6-(methoxycarbonyl) tetrahydro-2H-pyran-2-yl)-3-(2-(4-chlorophenyl) acetamido) propane-1,2-diyl diacetate (5) as yellow solid. Yield: 0.7 g, 49%; LCMS (ESI±) m/z 1106.01 [M+H]⁺.

### Synthesis of (2R,4S,5R,6R)-6-((1R,2R)-3-(2-(4-chlorophenyl) acetamido)-1,2-dihydroxypropyl)-2-((2,2-dimethyl-4,20-dioxo-3,8,11,14,17-pentaoxa-5,21-diazaheptacosan-27-yl) oxy)-4-hydroxy-5-(2-hydroxyacetamido) tetrahydro-2H-pyran-2-carboxylic acid (6)

To a stirred solution of (1R,2R)-1-((2R,3R,4S,6R)-4-acetoxy-3-(2-acetoxyacetamido)-6-((2,2-dimethyl-4,20-dioxo-3,8,11,14,17-pentaoxa-5,21-diazaheptacosan-27-yl) oxy)-6-(methoxycarbonyl) tetrahydro-2H-pyran-2-yl)-3-(2-(4-chlorophenyl) acetamido) propane-1,2-diyl diacetate (5) (0.7 g, 0.63 mmol) in tetrahydrofuran (7 mL) was added aq. solution of lithium hydroxide monohydrate (159 mg, 3.80 mmol) at 0° C. The reaction mixture was stirred at room temperature for 16 h. After completion, the reaction mixture was treated with acidic resin (Dowex 50, H⁺) to pH˜6 and the suspension was filtered, filtrate was concentrated, to afford (2R,4S,5R,6R)-6-((1R,2R)-3-(2-(4-chlorophenyl) acetamido)-1,2-dihydroxypropyl)-2-((2,2-dimethyl-4,20-dioxo-3,8,11,14,17-pentaoxa-5,21-diazaheptacosan-27-yl) oxy)-4-hydroxy-5-(2-hydroxyacetamido) tetrahydro-2H-pyran-2-carboxylic acid (6) as off white solid. Yield: 0.2 g, 34%; LCMS (ESI±) m/z 924.0 [M+H]⁺.

### Synthesis of (2R,4S,5R,6R)-2-((1-amino-15-oxo-3,6,9,12-tetraoxa-16-azadocosan-22-yl) oxy)-6-((1R,2R)-3-(2-(4-chlorophenyl) acetamido)-1,2-dihydroxypropyl)-4-hydroxy-5-(2-hydroxyacetamido) tetrahydro-2H-pyran-2-carboxylic acid (7)

To a stirred solution of (2R,4S,5R,6R)-6-((1R,2R)-3-(2-(4-chlorophenyl) acetamido)-1,2-dihydroxypropyl)-2-((2,2-dimethyl-4,20-dioxo-3,8,11,14,17-pentaoxa-5,21-diazaheptacosan-27-yl) oxy)-4-hydroxy-5-(2-hydroxyacetamido) tetrahydro-2H-pyran-2-carboxylic acid (6) (0.35 g, 0.37 mmol) in anhydrous dichloromethane (3.5 mL) was added trifluoroacetic acid (1.75 mL) at 0° C. The reaction mixture was stirred at room temperature for 2 h. After completion, the mixture was concentrated, and the crude residue obtained was suspended in methanol and neutralized with tetra alkyl ammonium carbonate (polymer-supported). The reaction mixture was filtered and the filtrate was concentrated to afford (2R,4S,5R,6R)-2-((1-amino-15-oxo-3,6,9,12-tetraoxa-16-azadocosan-22-yl) oxy)-6-((1R,2R)-3-(2-(4-chlorophenyl) acetamido)-1,2-dihydroxypropyl)-4-hydroxy-5-(2-hydroxyacetamido) tetrahydro-2H-pyran-2-carboxylic acid (7) as yellow solid. Yield:280 mg, 90%; LCMS (ESI±) m/z 823.22 [M+H]⁺.

### Synthesis of (2R,4S,5R,6R)-6-((1R,2R)-3-(2-(4-chlorophenyl) acetamido)-1,2-dihydroxypropyl)-2-((2,18-dioxo-6,9,12,15-tetraoxa-3,19-diazapentacosan-25-yl) oxy)-4-hydroxy-5-(2-hydroxyacetamido) tetrahydro-2H-pyran-2-carboxylic acid (244)

To a stirred solution of (2R,4S,5R,6R)-2-((1-amino-15-oxo-3,6,9,12-tetraoxa-16-azadocosan-22-yl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (7) (100 mg, 0.12 mmol) in N,N-dimethyl formamide (1 mL) was added 2,5-dioxopyrrolidin-1-yl acetate (8) (18 mg, 0.11 mmol) at 0° C. The resulting reaction mixture was stirred at room temperature for 1 h. After completion, the reaction mixture was purified by C18 prep-HPLC, by using acetonitrile and water (+0.05%, TFA) as eluents to afford (2R,4S,5R,6R)-6-((1R,2R)-3-(2-(4-chlorophenyl) acetamido)-1,2-dihydroxypropyl)-2-((2,18-dioxo-6,9,12,15-tetraoxa-3,19-diazapentacosan-25-yl) oxy)-4-hydroxy-5-(2-hydroxyacetamido) tetrahydro-2H-pyran-2-carboxylic acid (244) as white solid. Yield: 13 mg, 12%; LCMS (ESI±) m/z 864.38 [M+H]⁺. ¹H NMR (400 MHz, MeOD) δ 7.31 (s, 4H), 4.04 (s, 2H), 3.89-3.83 (m, 1H), 3.74-3.62 (m, 16H), 3.61-3.52 (m, 4H), 3.46-3.34 (m, 4H), 3.29-3.15 (m, 5H), 2.74 (dd, J=12.3, 4.0 Hz, 1H), 2.45 (t, J=6.2 Hz, 3H), 1.96 (s, 3H), 1.75 (t, J=11.9 Hz, 1H), 1.59-1.47 (m, 4H), 1.37 (d, J=5.9 Hz, 4H).

### (2R,4S,5R,6R)-6-((1R,2R)-3-(2-(4-chlorophenyl) acetamido)-1,2-dihydroxypropyl)-2-((1-(2,5-dioxo-2,5-dihydro-1H-pyrrol-1-yl)-3,19-dioxo-7,10,13,16-tetraoxa-4,20-diazahexacosan-26-yl) oxy)-4-hydroxy-5-(2-hydroxyacetamido) tetrahydro-2H-pyran-2-carboxylic acid (Cpd. No. 244M)

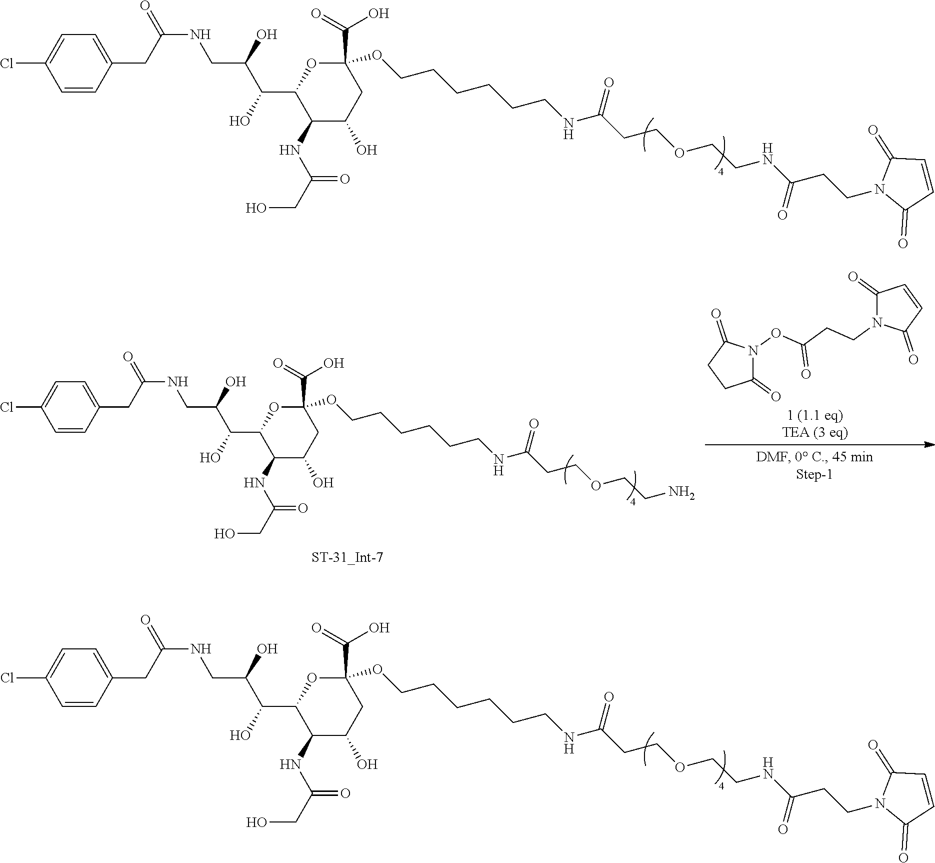

### Synthesis of (2R,4S,5R,6R)-6-((1R,2R)-3-(2-(4-chlorophenyl) acetamido)-1,2-dihydroxypropyl)-2-((1-(2,5-dioxo-2,5-dihydro-1H-pyrrol-1-yl)-3,19-dioxo-7,10,13,16-tetraoxa-4,20-diazahexacosan-26-yl) oxy)-4-hydroxy-5-(2-hydroxyacetamido) tetrahydro-2H-pyran-2-carboxylic acid (244M)

To a stirred solution of (2R,4S,5R,6R)-2-((1-amino-15-oxo-3,6,9,12-tetraoxa-16-azadocosan-22-yl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (ST-31_Int-7) (180 mg, 0.21 mmol) and 2,5-dioxopyrrolidin-1-yl 3-(2,5-dioxo-2,5-dihydro-1H-pyrrol-1-yl)propanoate (1) (54.4 mg, 0.19 mmol) in N,N-dimethyl formamide (1.8 mL) was added triethylamine (0.05 ml, 0.327 mmol) at 0° C. The resulting reaction mixture was stirred at 0° C. for 45 min. After completion, the reaction mixture was purified by C18 prep-HPLC using acetonitrile and water (+0.05%, TFA) as eluents to afford (2R,4S,5R,6R)-6-((1R,2R)-3-(2-(4-chlorophenyl) acetamido)-1,2-dihydroxypropyl)-2-((1-(2,5-dioxo-2,5-dihydro-1H-pyrrol-1-yl)-3,19-dioxo-7,10,13,16-tetraoxa-4,20-diazahexacosan-26-yl) oxy)-4-hydroxy-5-(2-hydroxyacetamido) tetrahydro-2H-pyran-2-carboxylic acid (244M), Yield: 40 mg, 19%; LCMS (ESI±) m/z 974.0 [M+H]⁺. ¹H NMR (400 MHz, MeOD-d4) δ 7.93 (d, J=8 Hz, 1H), 7.32 (s, 4H), 6.84 (s, 2H), 4.05 (s, 2H), 3.95-3.59 (m, 23H), 3.58-3.49 (m, 6H), 3.35-3.21 (m, 3H), 2.74 (dd, J=12.8, 4.2 Hz, 1H), 2.48 (dt, J=14.3, 6.5 Hz, 5H), 1.76 (t, J=12.0 Hz, 1H), 1.60-1.48 (m, 4H), 1.38 (d, J=4 Hz, 4H).

### (2R,4S,5R,6R)-6-((1R,2R)-3-(2-(4-chlorophenyl) acetamido)-1,2-dihydroxypropyl)-2-((6-((3,4-dioxo-2-((2-oxo-6,9,12,15-tetraoxa-3-azaheptadecan-17-yl)amino) cyclobut-1-en-1-yl) amino)hexyl)oxy)-5-(2-fluoroacetamido)-4-hydroxytetrahydro-2H-pyran-2-carboxylic acid (Cpd. 245)

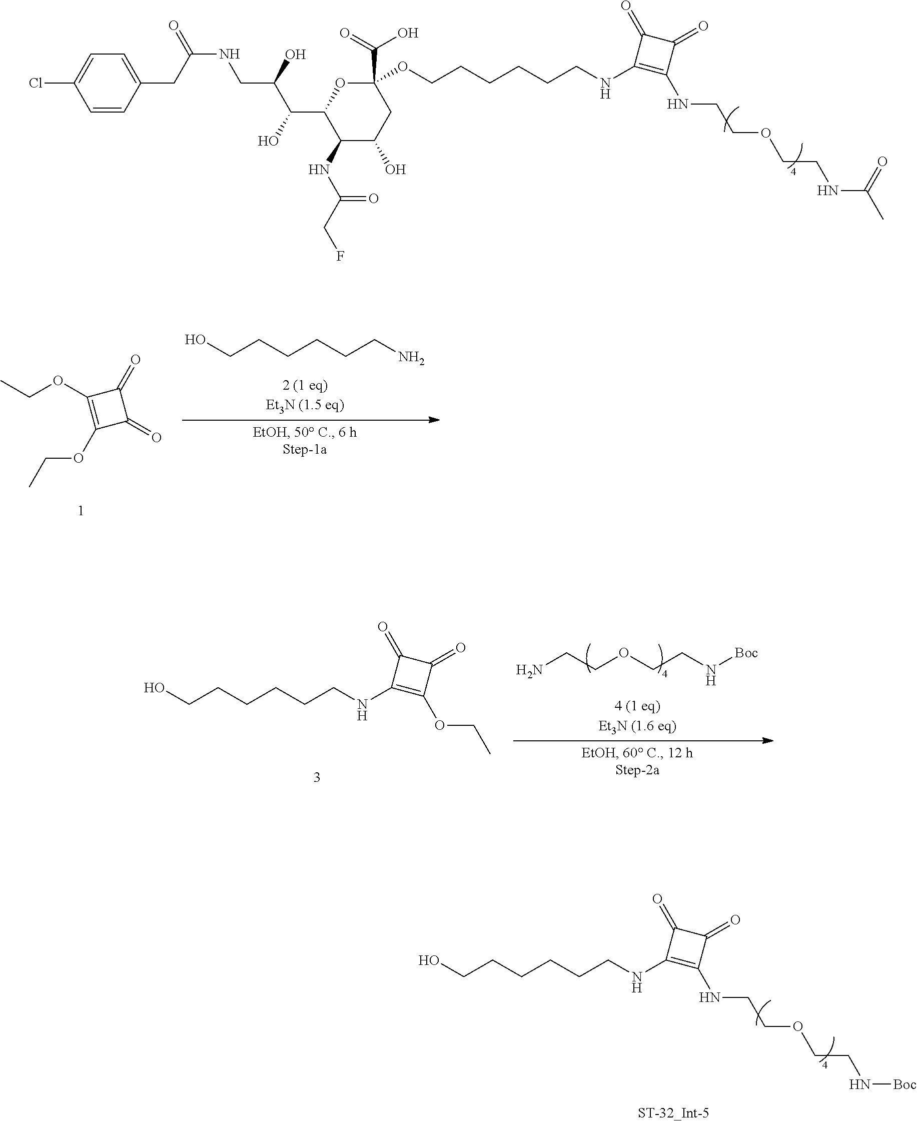

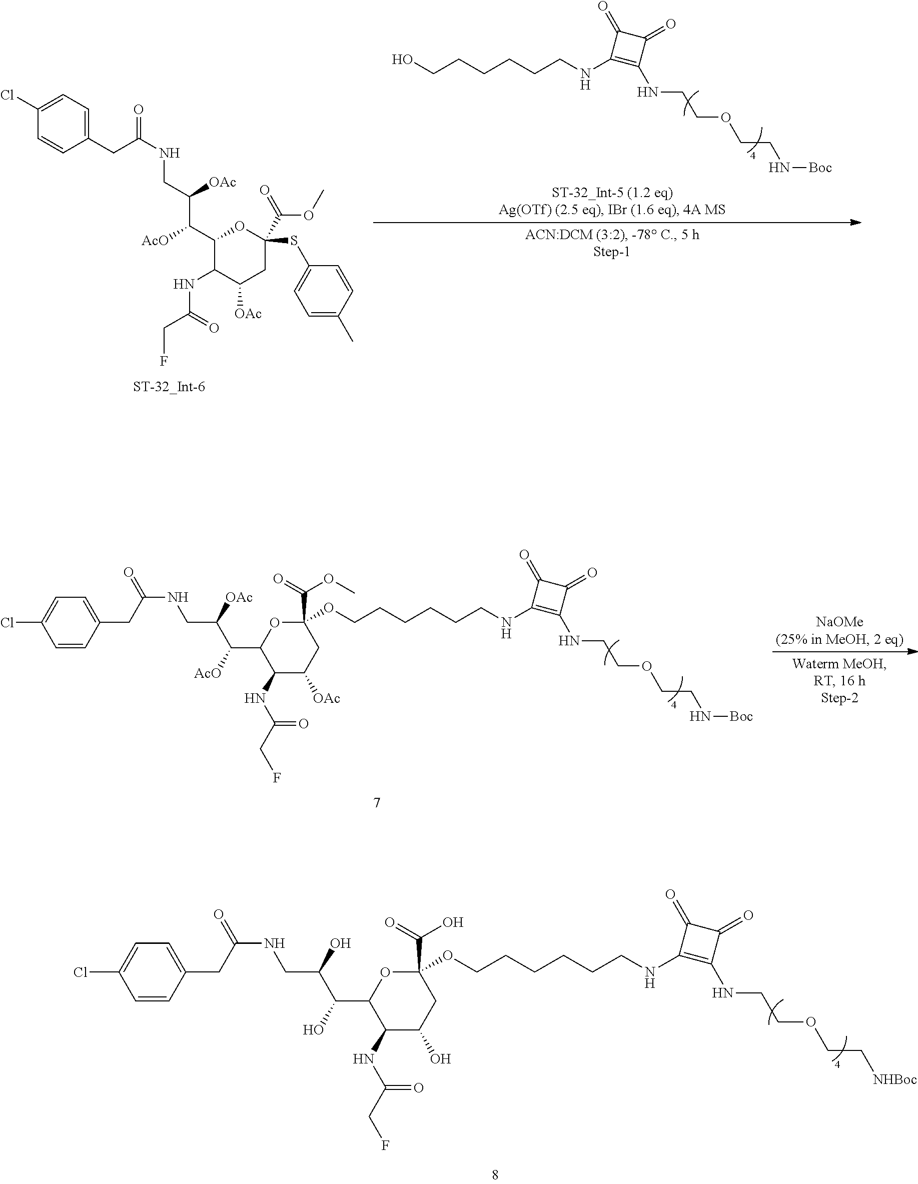

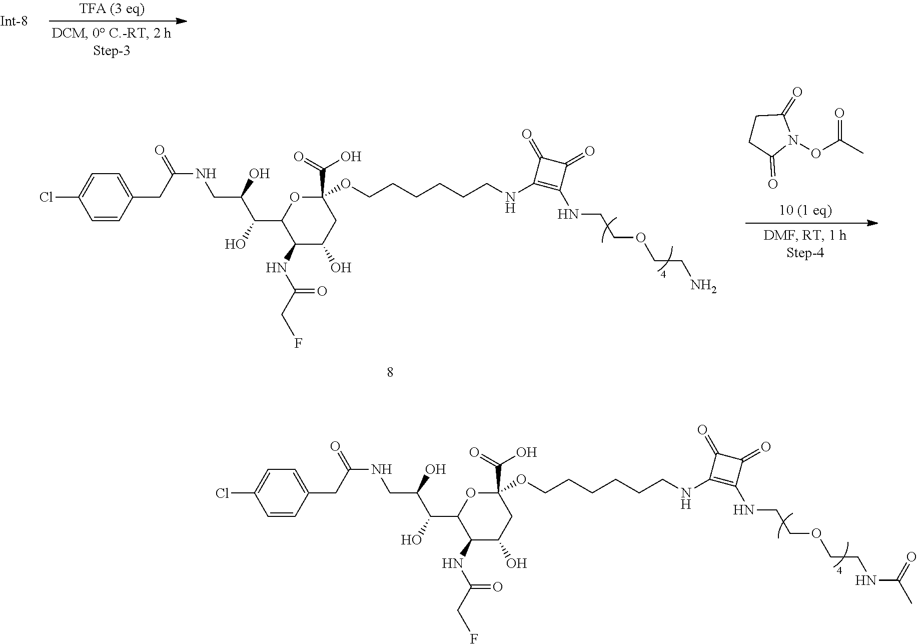

### Synthesis of 3-ethoxy-4-((6-hydroxyhexyl)amino) cyclobut-3-ene-1,2-dione (3)

To a stirred solution of 3,4-diethoxycyclobut-3-ene-1,2-dione (1) (5 g, 29.41 mmol) and 6-aminohexan-1-ol (2) (1.72 g, 14.7 mmol) in ethanol (50 mL) was added triethyl amine (6.36 ml, 44.11 mmol) and the reaction mixture was heated at 50° C. for 6 h. After completion, the reaction mixture was concentrated and the crude residue was purified by silica gel column chromatography, by using ethyl acetate and hexane as eluents to afford 3-ethoxy-4-((6-hydroxyhexyl)amino) cyclobut-3-ene-1,2-dione (3) as yellow solid. Yield: 5 g, 70.62%; LCMS (ESI±) m/z 242.2 [M+H]⁺.

### Synthesis of tert-butyl (14-((2-((6-hydroxyhexyl)amino)-3,4-dioxocyclobut-1-en-1-yl)amino)-3,6,9,12-tetraoxatetradecyl) carbamate (5)

To a stirred solution of 3-ethoxy-4-((6-hydroxyhexyl)amino)cyclobut-3-ene-1,2-dione (3) (5 g, 20.72 mmol) in ethanol (50 mL) were added tert-butyl (14-amino-3,6,9,12-tetraoxatetradecyl)carbamate (3) (6.97 g, 20.72 mmol), triethylamine (4.49 ml, 31.12 mmol) and the resulting reaction mixture was stirred at 60° C. for 12 h. After completion, the reaction mixture was concentrated, the crude residue was purified by C18 reverse phase column chromatography using acetonitrile and water (+0.05%, TFA) as eluents to afford tert-butyl (14-((2-((6-hydroxyhexyl)amino)-3,4-dioxocyclobut-1-en-1-yl)amino)-3,6,9,12-tetraoxatetradecyl) carbamate (ST-32_Int-5) as white solid. Yield: 3 g, 27.27%; LCMS (ESI±) m/z 532.4 [M+H]⁺.

### Synthesis of (1R,2R)-1-((2R,3R,4S,6R)-4-acetoxy-6-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-3-(2-fluoroacetamido)-6-(methoxycarbonyl) tetrahydro-2H-pyran-2-yl)-3-(2-(4-chlorophenyl) acetamido) propane-1,2-diyl diacetate (7)

To a stirred solution of (1R,2R)-1-((2R,3R,4S,6R)-4-acetoxy-3-(2-fluoroacetamido)-6-(methoxycarbonyl)-6-(p-tolylthio)tetrahydro-2H-pyran-2-yl)-3-(2-(4-chlorophenyl)acetamido)propane-1,2-diyl diacetate (ST-32_Int-6) (1.5 g, 2.06 mmol) and tert-butyl (14-((2-((6-hydroxyhexyl)amino)-3,4-dioxocyclobut-1-en-1-yl)amino)-3,6,9,12-tetraoxatetradecyl)carbamate (ST-32_Int-5) (1.31 g, 2.48 mmol), in anhydrous acetonitrile (18 mL) and anhydrous dichloromethane (12 mL) were added 4 Å powdered molecular sieves (4 g) and Ag(OTf) (1.37 g, 5.17 mmol) under nitrogen atmosphere. The reaction mixture was stirred for 30 min. and cooled to −78° C. A solution of iodine monobromide (0.63 g, 3.10 mmol) in dichloromethane (4 ml) was added dropwise. The reaction mixture was stirred at the same temperature for 5 h. After completion, the reaction mixture was diluted with dichloromethane, quenched with triethyl amine (up to pH=7) then filtered. The filtrate was washed with 10% aqueous aq. sodium thio-sulfate, dried over sodium sulfate, concentrated. The crude residue obtained was purified by C18 reverse phase column chromatography using acetonitrile and water (0.05%, TFA) as eluents to afford (1R,2R)-1-((2R,3R,4S,6R)-4-acetoxy-6-((6-((2-((2,2-dimethyl-4-oxo-3,8,11,14,17-pentaoxa-5-azanonadecan-19-yl)amino)-3,4-dioxocyclobut-1-en-1-yl) amino)hexyl)oxy)-3-(2-fluoroacetamido)-6-(methoxycarbonyl) tetrahydro-2H-pyran-2-yl)-3-(2-(4-chlorophenyl) acetamido) propane-1,2-diyl diacetate (7) as yellow solid. Yield: 1 g, 42.73%; LCMS (ESI±) m/z 1133.9 [M+H]⁺.

### Synthesis of (2R,4S,5R,6R)-6-((1R,2R)-3-(2-(4-chlorophenyl) acetamido)-1,2-dihydroxypropyl)-2-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-5-(2-fluoroacetamido)-4-hydroxytetrahydro-2H-pyran-2-carboxylic acid (8)

To a stirred solution of (1R,2R)-1-((2R,3R,4S,6R)-4-acetoxy-6-((6-((2-((2,2-dimethyl-4-oxo-3,8,11,14,17-pentaoxa-5-azanonadecan-19-yl)amino)-3,4-dioxocyclobut-1-en-1-yl)amino) hexyl)oxy)-3-(2-fluoroacetamido)-6-(methoxycarbonyl)tetrahydro-2H-pyran-2-yl)-3-(2-(4-chlorophenyl)acetamido)propane-1,2-diyl diacetate (7) (800 mg, 0.7 mmol) in methanol (9 mL) and water (1 mL) was added 25% sodium methoxide in methanol (0.33 mL, 1.41 mmol) at 0° C., and the reaction mixture was stirred at room temperature for 16 h. After completion, the reaction mixture was acidified with acidic resin (Dowex 50H⁺) to pH˜6, the suspension was filtered, and the filtrate was concentrated. The crude residue obtained was purified by C18 reverse phase column chromatography using acetonitrile and water (+0.05%, TFA) as eluents to afford (2R,4S,5R,6R)-6-((1R,2R)-3-(2-(4-chlorophenyl) acetamido)-1,2-dihydroxypropyl)-2-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-5-(2-fluoroacetamido)-4-hydroxytetrahydro-2H-pyran-2-carboxylic acid (8) as off white solid. Yield: 0.2 g, 28.53%; LCMS (ESI±) m/z 994.8 [M+H]⁺.

### Synthesis of (2R,4S,5R,6R)-2-((6-((2-((14-amino-3,6,9,12-tetraoxatetradecyl)amino)-3,4-dioxocyclobut-1-en-1-yl)amino)hexyl)oxy)-6-((1R,2R)-3-(2-(4-chlorophenyl) acetamido)-1,2-dihydroxypropyl)-5-(2-fluoroacetamido)-4-hydroxytetrahydro-2H-pyran-2-carboxylic acid (9)

To a stirred solution of (2R,4S,5R,6R)-6-((1R,2R)-3-(2-(4-chlorophenyl) acetamido)-1,2-dihydroxypropyl)-2-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-5-(2-fluoroacetamido)-4-hydroxytetrahydro-2H-pyran-2-carboxylic acid (8) (0.5 g, 0.5 mmol) in anhydrous dichloromethane (5 mL) was added trifluoro acetic acid (1 mL), the reaction mixture was stirred at room temperature for 2 h. After completion, the mixture was concentrated under reduced pressure and the crude material was suspended in methanol and neutralized with tetra-alkyl ammonium carbonate (polymer-bound). Reaction mixture was filtered and filtrate was concentrate to get (2R,4S,5R,6R)-2-((6-((2-((14-amino-3,6,9,12-tetraoxatetradecyl)amino)-3,4-dioxocyclobut-1-en-1-yl)amino)hexyl)oxy)-6-((1R,2R)-3-(2-(4-chlorophenyl) acetamido)-1,2-dihydroxypropyl)-5-(2-fluoroacetamido)-4-hydroxytetrahydro-2H-pyran-2-carboxylic acid (9) as yellow solid. Yield-200 mg, 62.03%; LCMS (ESI) m/z 894.7 [M+H]⁺.

### Synthesis of (2R,4S,5R,6R)-6-((1R,2R)-3-(2-(4-chlorophenyl) acetamido)-1,2-dihydroxypropyl)-2-((6-((3,4-dioxo-2-((2-oxo-6,9,12,15-tetraoxa-3-azaheptadecan-17-yl)amino) cyclobut-1-en-1-yl)amino)hexyl)oxy)-5-(2-fluoroacetamido)-4-hydroxytetrahydro-2H-pyran-2-carboxylic acid (245)

To a stirred solution of (2R,4S,5R,6R)-2-((6-((2-((14-amino-3,6,9,12-tetraoxatetradecyl)amino)-3,4-dioxocyclobut-1-en-1-yl)amino)hexyl)oxy)-6-((1R,2R)-3-(2-(4-chlorophenyl)acetamido)-1,2-dihydroxypropyl)-5-(2-fluoroacetamido)-4-hydroxytetrahydro-2H-pyran-2-carboxylic acid (9) (55 mg, 0.061 mmol) in N,N-dimethylformamide (2 mL) was added 2,5-dioxopyrrolidin-1-yl acetate (10) (9.6 mg, 0.06 mmol) at 0° C. The resulting reaction mixture was stirred at room temperature for 1 h. After completion, the reaction mixture was purified by Prep HPLC purification by using acetonitrile and water (+0.05%, TFA) as eluents to afford (2R,4S,5R,6R)-6-((1R,2R)-3-(2-(4-chlorophenyl) acetamido)-1,2-dihydroxypropyl)-2-((6-((3,4-dioxo-2-((2-oxo-6,9,12,15-tetraoxa-3-azaheptadecan-17-yl)amino) cyclobut-1-en-1-yl)amino) hexyl)oxy)-5-(2-fluoroacetamido)-4-hydroxytetrahydro-2H-pyran-2-carboxylic acid (245) as white solid. Yield: 30 mg, 52.17%; LCMS (ESI±) m/z 935.0 [M+H]⁺. ¹H NMR (400 MHz, MeOD) δ 7.32 (s, 4H), 4.79 (s, 1H), 3.92-3.67 (m, 8H), 3.72-3.63 (m, 18H), 3.56-3.54 (m, 4H), 3.48 (t, J=7.4 Hz, 1H), 3.44-3.35 (m, 3H), 2.81 (dd, J=12.8 Hz, 1H), 1.98 (s, 3H), 1.65-1.57 (m, 5H), 1.41 (d, J=5.4 Hz, 4H).

### (2R,4S,5R,6R)-6-((1R,2R)-3-(2-(4-chlorophenyl) acetamido)-1,2-dihydroxypropyl)-2-((6-((2-((18-(2,5-dioxo-2,5-dihydro-1H-pyrrol-1-yl)-16-oxo-3,6,9,12-tetraoxa-15-azaoctadecyl) amino)-3,4-dioxocyclobut-1-en-1-yl)amino)hexyl)oxy)-5-(2-fluoroacetamido)-4-hydroxytetrahydro-2H-pyran-2-carboxylic acid (Cpd. 245M)

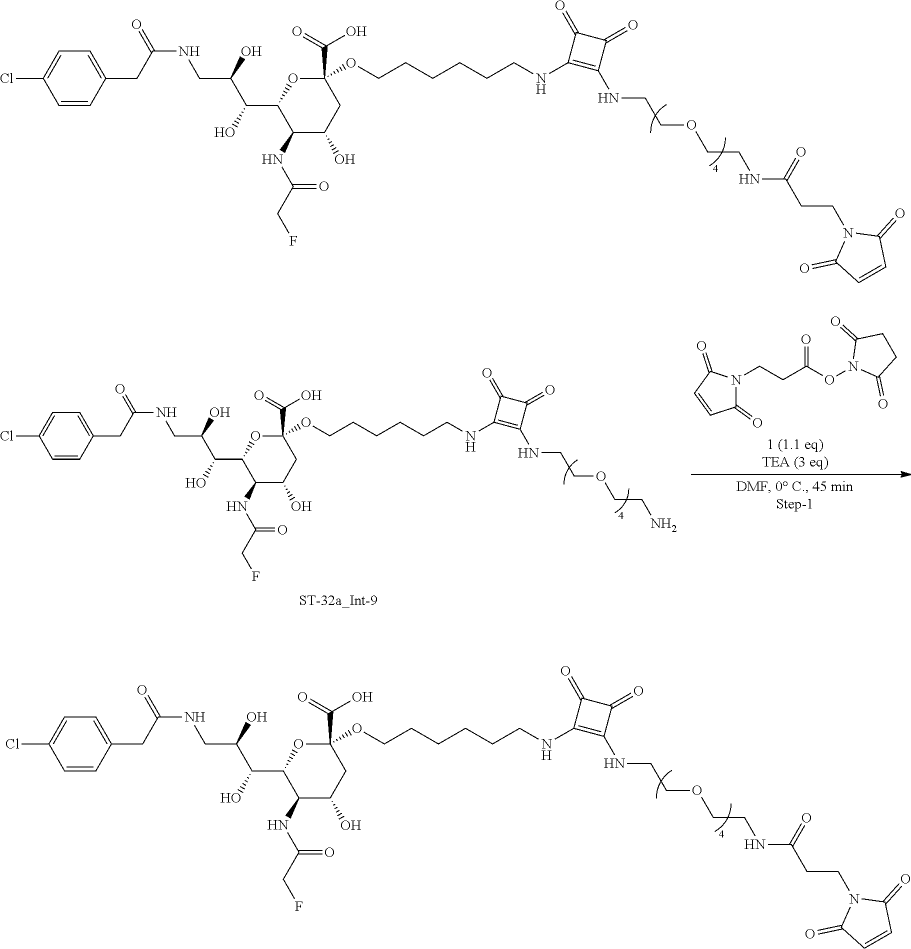

### Synthesis of (2R,4S,5R,6R)-6-((1R,2R)-3-(2-(4-chlorophenyl) acetamido)-1,2-dihydroxypropyl)-2-((6-((2-((18-(2,5-dioxo-2,5-dihydro-1H-pyrrol-1-yl)-16-oxo-3,6,9,12-tetraoxa-15-azaoctadecyl)amino)-3,4-dioxocyclobut-1-en-1-yl)amino)hexyl)oxy)-5-(2-fluoroacetamido)-4-hydroxytetrahydro-2H-pyran-2-carboxylic acid (245M)

To a stirred solution of (2R,4S,5R,6R)-5-acetamido-2-((6-((2-((14-amino-3,6,9,12-tetraoxatetradecyl)amino)-3,4-dioxocyclobut-1-en-1-yl)amino)hexyl)oxy)-6-((1R,2R)-3-(2-(4-chlorophenyl)acetamido)-1,2-dihydroxypropyl)-4-hydroxytetrahydro-2H-pyran-2-carboxylic acid (245_Int-9) (110 mg, 0.12 mmol) and 2,5-dioxopyrrolidin-1-yl 3-(2,5-dioxo-2,5-dihydro-1H-pyrrol-1-yl)propanoate (1) (36 mg, 0.13 mmol) in N,N-dimethylformamide (2 mL) was added triethylamine (0.05 ml, 0.36 mmol) at 0° C., the resulting reaction mixture was stirred for 45 min at the same temperature. After completion, the crude reaction mixture was purified by C18 prep-HPLC using acetonitrile and water (+0.05%, TFA) as eluents to afford (2R,4S,5R,6R)-6-((1R,2R)-3-(2-(4-chlorophenyl) acetamido)-1,2-dihydroxypropyl)-2-((6-((2-((18-(2,5-dioxo-2,5-dihydro-1H-pyrrol-1-yl)-16-oxo-3,6,9,12-tetraoxa-15-azaoctadecyl)amino)-3,4-dioxocyclobut-1-en-1-yl) amino)hexyl)oxy)-5-(2-fluoroacetamido)-4-hydroxytetrahydro-2H-pyran-2-carboxylic acid (245M) as white solid. Yield: 10 mg, 7.81%; LCMS (ESI±) m/z 1044.8 [M+H]⁺. ¹H NMR (400 MHz, MeOD) δ 7.32 (s, 4H), 6.85 (s, 2H), 4.78 (d, J=2.4 Hz, 2H), 3.95-3.59 (m, 18H), 3.58-3.49 (m, 8H), 3.47-3.36 (m, 2H), 3.35-3.21 (m, 6H), 3.19 (dt, J=6.9, 3.4 Hz, 1H), 2.74 (dd, J=12.8, 4.2 Hz, 1H), 2.48 (dt, J=14.3, 6.5 Hz, 2H), 1.76 (t, J=12.0 Hz, 1H), 1.60-1.48 (m, 4H), 1.40 (d, J=3.2 Hz, 4H).

### (2R,4S,5R,6R)-6-((1R,2R)-3-(2-(4-chlorophenyl) acetamido)-1,2-dihydroxypropyl)-2-((6-((2-((16,32-dioxo-36-((3aR,4R,6aS)-2-oxohexahydro-1H-thieno[3,4-d] imidazol-4-yl)-3,6,9,12,19,22,25,28-octaoxa-15,31-diazahexatriacontyl)amino)-3,4-dioxocyclobut-1-en-1-yl)amino)hexyl)oxy)-5-(2-fluoroacetamido)-4-hydroxytetrahydro-2H-pyran-2-carboxylic acid (Cpd. 245B)

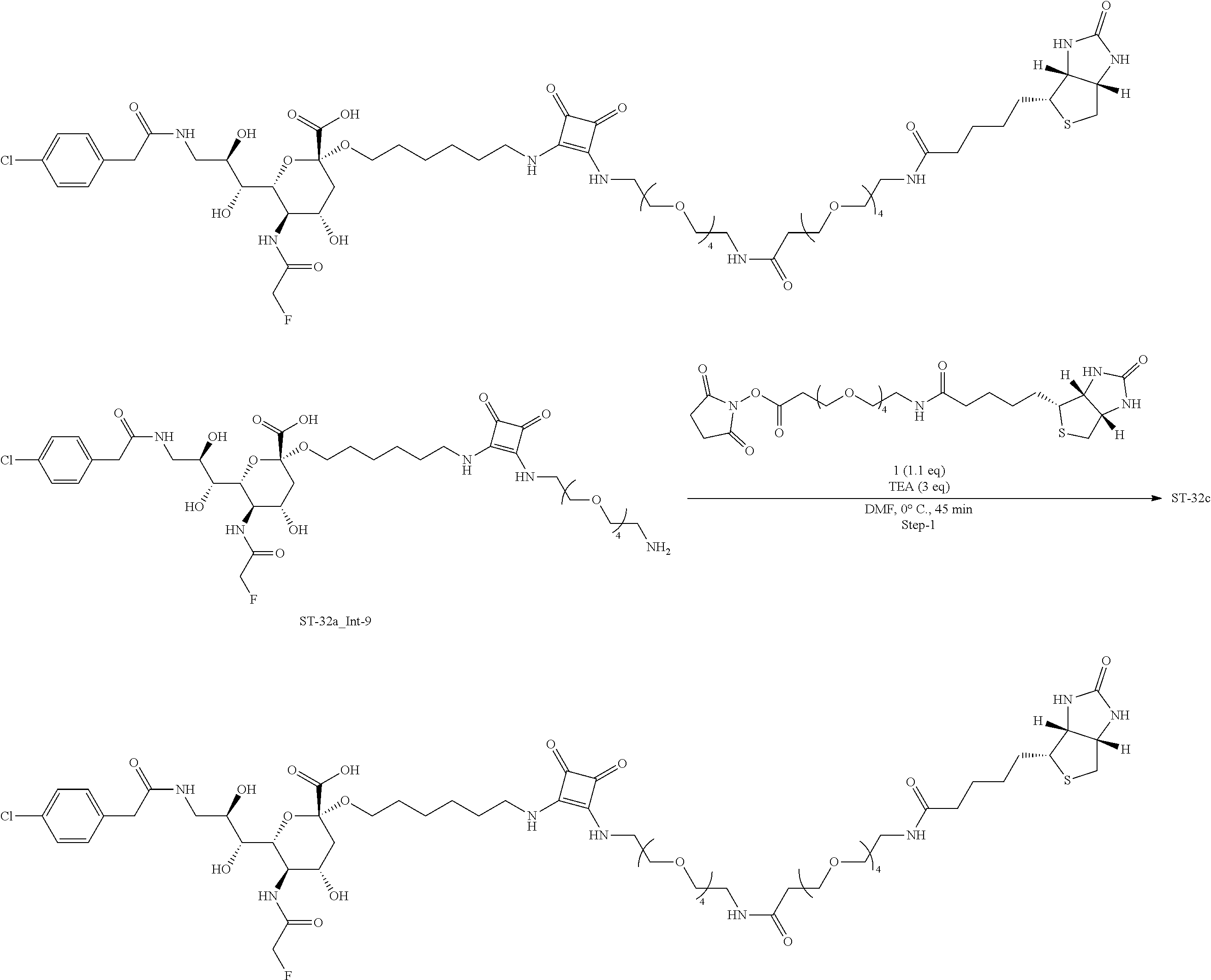

### Synthesis of (2R,4S,5R,6R)-6-((1R,2R)-3-(2-(4-chlorophenyl) acetamido)-1,2-dihydroxypropyl)-2-((6-((2-((16,32-dioxo-36-((3aR,4R,6aS)-2-oxohexahydro-1H-thieno[3,4-d] imidazol-4-yl)-3,6,9,12,19,22,25,28-octaoxa-15,31-diazahexatriacontyl)amino)-3,4-dioxocyclobut-1-en-1-yl) amino)hexyl)oxy)-5-(2-fluoroacetamido)-4-hydroxytetrahydro-2H-pyran-2-carboxylic acid (245B)

To a stirred solution of (2R,4S,5R,6R)-2-((6-((2-((14-amino-3,6,9,12-tetraoxatetradecyl)amino)-3,4-dioxocyclobut-1-en-1-yl)amino)hexyl)oxy)-6-((1R,2R)-3-(2-(4-chlorophenyl)acetamido)-1,2-dihydroxypropyl)-5-(2-fluoroacetamido)-4-hydroxytetrahydro-2H-pyran-2-carboxylic acid (245_Int-9) (50 mg, 0.05 mmol) in N,N-dimethylformamide (1 mL) was added 2,5-dioxopyrrolidin-1-yl 17-oxo-21-((3aR,4R,6aS)-2-oxohexahydro-1H-thieno[3,4-d]imidazol-4-yl)-4,7,10,13-tetraoxa-16-azahenicosanoate (1) (36 mg, 0.06 mmol) and triethylanine (0.02 ml, 0.16 mmol) at 0° C. The resulting reaction mixture was stirred at 0° C. for 45 min. After completion, the reaction mixture was purified by C18 prep-HPLC using acetonitrile and water (+0.05%, TFA) as eluents to afford (2R,4S,5R,6R)-6-((1R,2R)-3-(2-(4-chlorophenyl) acetamido)-1,2-dihydroxypropyl)-2-((6-((2-((16,32-dioxo-36-((3aR,4R,6aS)-2-oxohexahydro-1H-thieno[3,4-d] imidazol-4-yl)-3,6,9,12,19,22,25,28-octaoxa-15,31-diazahexatriacontyl) amino)-3,4-dioxocyclobut-1-en-1-yl)amino)hexyl)oxy)-5-(2-fluoroacetamido)-4-hydroxytetrahydro-2H-pyran-2-carboxylic acid (245B) as white solid. Yield: 32 mg, 41.82%; LCMS (ESI±) m/z 1366.2 [M+H]⁺. ¹H NMR (400 MHz, MeOD) δ 7.31 (s, 4H), 4.77 (d, J=1.4 Hz, 2H), 3.97-3.69 (m, 40H), 3.69-3.54 (m, 10H), 3.51 (t, J=5.4 Hz, 2H), 3.46-3.35 (m, 12H), 3.33-3.21 (m, 9H), 2.71 (dd, J=12.7, 4.5 Hz, 1H), 2.48 (t, J=6.9 Hz, 2H), 1.80-1.69 (m, 4H), 1.56 (s, 3H), 1.73-1.39 (m, 10H).

### (2R,4S,5R,6R)-6-((1R,2R)-3-(2-([1,1′-biphenyl]-4-yl) acetamido)-1,2-dihydroxypropyl)-2-((6-((3,4-dioxo-2-((2-oxo-6,9,12,15-tetraoxa-3-azaheptadecan-17-yl)amino) cyclobut-1-en-1-yl)amino)hexyl)oxy)-5-(2-fluoroacetamido)-4-hydroxytetrahydro-2H-pyran-2-carboxylic acid (Cpd. 21)

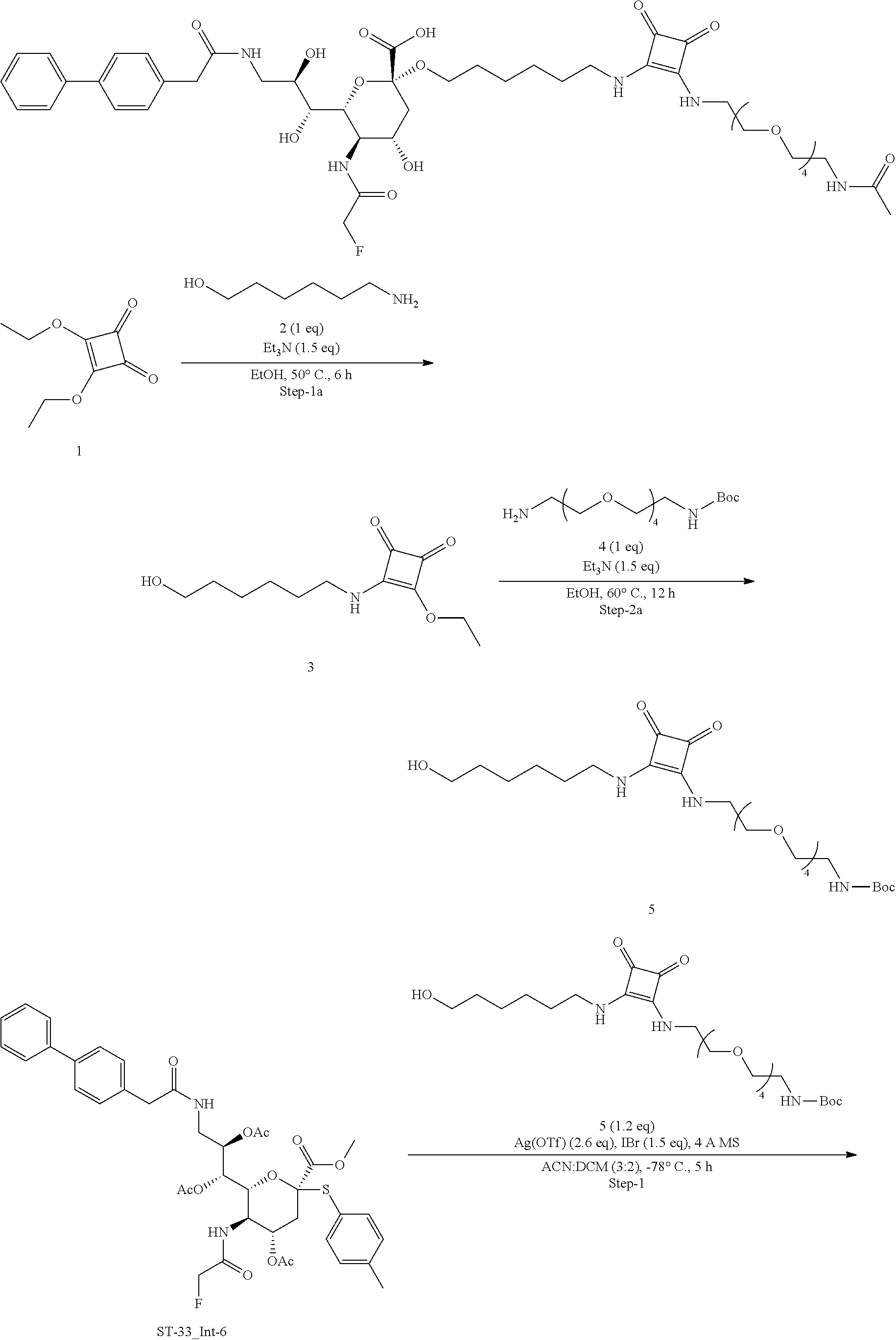

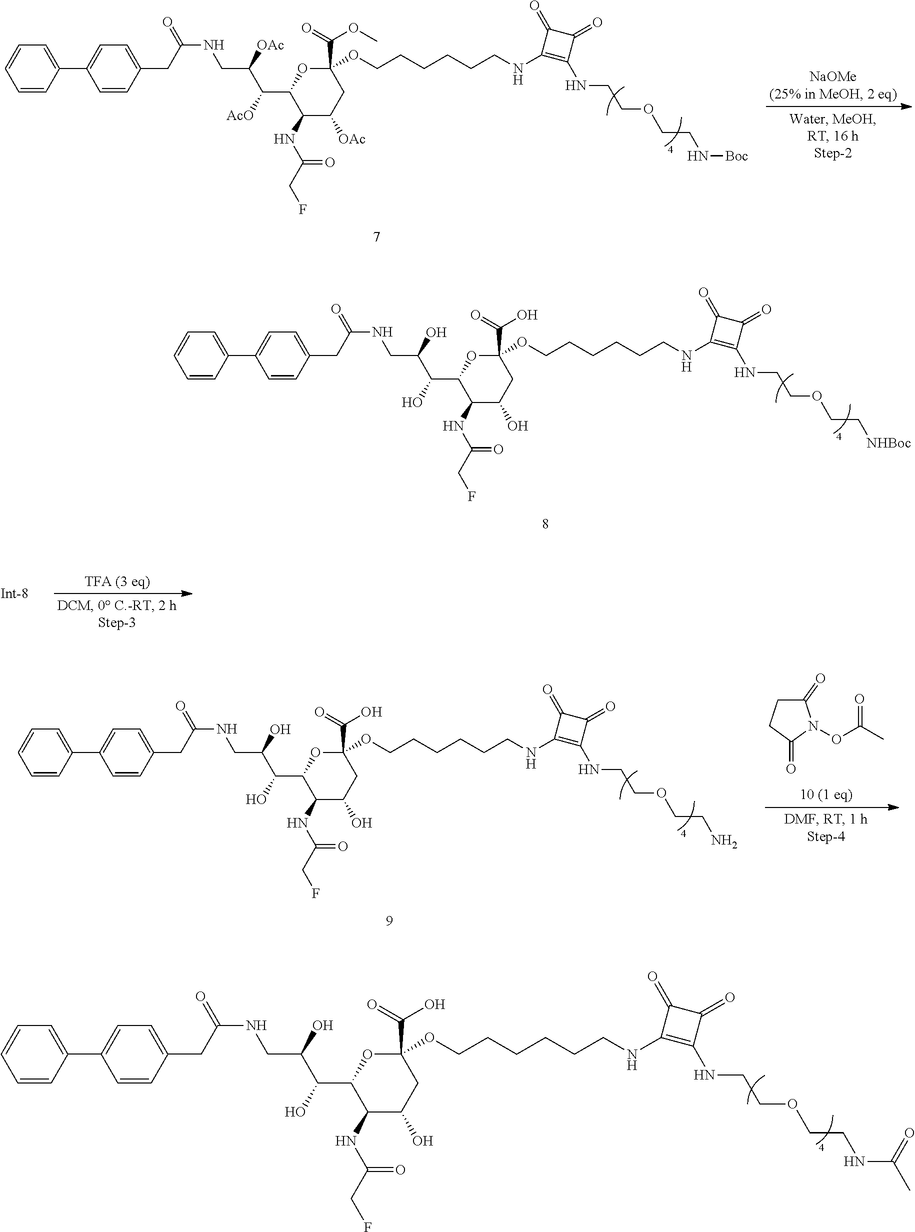

### Synthesis of 3-ethoxy-4-((6-hydroxyhexyl)amino) cyclobut-3-ene-1,2-dione (3)

To a stirred solution of 3,4-diethoxycyclobut-3-ene-1,2-dione (1) (5 g, 29.41 mmol) and 6-aminohexan-1-ol (2) (1.72 g, 14.70 mmol) in ethanol (50 mL) was added triethyl amine (6.36 ml, 44.11 mmol) and the reaction mixture was stirred at 50° C. for 6 h. After completion, the reaction mixture was concentrated. The crude residue obtained was purified by silica gel column chromatography using ethyl acetate and hexane as eluents to afford 3-ethoxy-4-((6-hydroxyhexyl) amino) cyclobut-3-ene-1,2-dione (3) as a yellow solid. Yield: 5 g, 70.62%; LCMS (ESI±) m/z 242.2 [M+H]⁺.

### Synthesis of tert-butyl (14-((2-((6-hydroxyhexyl)amino)-3,4-dioxocyclobut-1-en-1-yl)amino)-3,6,9,12-tetraoxatetradecyl) carbamate (ST-33_Int-5)

To a stirred solution of 3-ethoxy-4-((6-hydroxyhexyl)amino)cyclobut-3-ene-1,2-dione (3) (5 g, 20.72 mmol) and tert-butyl (14-amino-3,6,9,12-tetraoxatetradecyl)carbamate (4) (6.97 g, 20.72 mmol) in ethanol (50 mL) was added triethylamine (4.49 ml, 31.12 mmol) and the resulting reaction mixture was stirred at 60° C. for 12 h. After completion, the reaction mixture was concentrated and the crude residue was purified by C18 reverse phase column chromatography using acetonitrile and water (+0.05%, TFA) as eluents to afford tert-butyl (14-((2-((6-hydroxyhexyl)amino)-3,4-dioxocyclobut-1-en-1-yl)amino)-3,6,9,12-tetraoxatetradecyl) carbamate (ST-33_Int-5) as white solid. Yield: 3.0 g, 27.27%; LCMS (ESI±) m/z 532.4 [M+H]⁺.

### Synthesis of (1R,2R)-3-(2-([1,1′-biphenyl]-4-yl) acetamido)-1-((2R,3R,4S,6R)-4-acetoxy-6-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-3-(2-fluoroacetamido)-6-(methoxycarbonyl) tetrahydro-2H-pyran-2-yl) propane-1,2-diyl diacetate (7)

To a stirred solution of (1R,2R)-3-(2-([1,1′-biphenyl]-4-yl)acetamido)-1-((2R,3R,4S,6R)-4-acetoxy-3-(2-fluoroacetamido)-6-(methoxycarbonyl)-6-(p-tolylthio)tetrahydro-2H-pyran-2-yl)propane-1,2-diyl diacetate (ST-33_Int-6) (2 g, 2.61 mmol) and tert-butyl (14-((2-((6-hydroxyhexyl)amino)-3,4-dioxocyclobut-1-en-1-yl)amino)-3,6,9,12-tetraoxatetradecyl)carbamate (5) (1.66 g, 3.13 mmol) in anhydrous acetonitrile (18 mL) and anhydrous dichloromethane (12 mL) were added 4 Å powdered molecular sieves (4 g) and Ag(OTf) (3.46 g, 6.52 mmol) under nitrogen atmosphere and stirred for 30 min at room temperature. The reaction mixture was cooled to −78° C. and a solution of iodine monobromide (0.80 g, 3.91 mmol) in DCM (4 ml) was added dropwise at the same temperature. The reaction mixture was continued at the same temperature for 5 h. After completion, the reaction mixture was diluted with dichloromethane (15 ml), quenched with triethyl amine (up to pH=7), filtered, the filtrate was washed with 10% aqueous sodium thiosulfate solution, dried over sodium sulfate, concentrated. The crude residue obtained was purified by C18 reverse phase column chromatography using acetonitrile and water (+0.05%, TFA) as eluents to afford (1R,2R)-3-(2-([1,1′-biphenyl]-4-yl) acetamido)-1-((2R,3R,4S,6R)-4-acetoxy-6-((6-((2-((2,2-dimethyl-4-oxo-3,8,11,14,17-pentaoxa-5-azanonadecan-19-yl)amino)-3,4-dioxocyclobut-1-en-1-yl)amino) hexyl)oxy)-3-(2-fluoroacetamido)-6-(methoxycarbonyl) tetrahydro-2H-pyran-2-yl) propane-1,2-diyl diacetate (7) as yellow solid. Yield: 1.5 g, 55.76%; LCMS (ESI±) m/z 1176.1 [M+H]⁺.

### Synthesis of (2R,4S,5R,6R)-6-((1R,2R)-3-(2-([1,1′-biphenyl]-4-yl) acetamido)-1,2-dihydroxypropyl)-2-((6-((2-((2,2-dimethyl-4-oxo-3,8,11,14,17-pentaoxa-5-azanonadecan-19-yl) amino)-3,4-dioxocyclobut-1-en-1-yl)amino)hexyl)oxy)-5-(2-fluoroacetamido)-4-hydroxytetrahydro-2H-pyran-2-carboxylic acid (8)

To a stirred solution of (1R,2R)-1-((2R,3R,4S,6R)-3-acetamido-4-acetoxy-6-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-6-(methoxycarbonyl) tetrahydro-2H-pyran-2-yl)-3-(2-(4-chlorophenyl) acetamido) propane-1,2-diyl diacetate (7) (1.5 mg, 1.27 mmol) in methanol (9 mL) and water (1 mL) was added 25% sodium methoxide in methanol (0.13 mL, 2.55 mmol) at 0° C. and the reaction mixture was stirred at room temperature for 16 h. After completion the reaction mixture was acidified with acidic resin (Dowex 50H⁺), filtered, the filtrate was concentrated. The crude residue was purified by C18 reverse phase column chromatography using acetonitrile and water (0.05%, TFA) as eluents to afford (2R,4S,5R,6R)-6-((1R,2R)-3-(2-([1,1′-biphenyl]-4-yl) acetamido)-1,2-dihydroxypropyl)-2-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-5-(2-fluoroacetamido)-4-hydroxytetrahydro-2H-pyran-2-carboxylic acid (8) as off white solid. Yield: 600 mg, 45.80%; LCMS (ESI±) m/z 1032.9 [M−H]⁻.

### Synthesis of (2R,4S,5R,6R)-6-((1R,2R)-3-(2-([1,1′-biphenyl]-4-yl) acetamido)-1,2-dihydroxypropyl)-2-((6-((2-((14-amino-3,6,9,12-tetraoxatetradecyl)amino)-3,4-dioxocyclobut-1-en-1-yl)amino)hexyl)oxy)-5-(2-fluoroacetamido)-4-hydroxytetrahydro-2H-pyran-2-carboxylic acid (9)

To a stirred solution of (2R,4S,5R,6R)-6-((1R,2R)-3-(2-([1,1′-biphenyl]-4-yl) acetamido)-1,2-dihydroxypropyl)-2-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-5-(2-fluoroacetamido)-4-hydroxytetrahydro-2H-pyran-2-carboxylic acid (8) (0.6 g, 0.58 mmol) in anhydrous dichloromethane (8 mL) was added trifluoro acetic acid (0.13 ml, 1.74 mmol) at 0° C. The reaction mixture was stirred at room temperature for 2 h. After completion, the reaction mixture was concentrated, crude was suspended in methanol and neutralized with tetra alkyl ammonium carbonate (polymer-bound). The reaction mixture was filtered and filtrate was concentrated to get (2R,4S,5R,6R)-6-((1R,2R)-3-(2-([1,1′-biphenyl]-4-yl) acetamido)-1,2-dihydroxypropyl)-2-((6-((2-((14-amino-3,6,9,12-tetraoxatetradecyl)amino)-3,4-dioxocyclobut-1-en-1-yl)amino)hexyl)oxy)-5-(2-fluoroacetamido)-4-hydroxytetrahydro-2H-pyran-2-carboxylic acid (9) as brown solid. Yield: 350 mg, 64.69%; LCMS (ESI±) m/z 935.8 [M+H]⁺.

### Synthesis of (2R,4S,5R,6R)-6-((1R,2R)-3-(2-([1,1′-biphenyl]-4-yl) acetamido)-1,2-dihydroxypropyl)-2-((6-((3,4-dioxo-2-((2-oxo-6,9,12,15-tetraoxa-3-azaheptadecan-17-yl) amino) cyclobut-1-en-1-yl)amino)hexyl)oxy)-5-(2-fluoroacetamido)-4-hydroxytetrahydro-2H-pyran-2-carboxylic acid (21)

To a stirred solution of (2R,4S,5R,6R)-6-((1R,2R)-3-(2-([1,1′-biphenyl]-4-yl)acetamido)-1,2-dihydroxypropyl)-2-((6-((2-((14-amino-3,6,9,12-tetraoxatetradecyl)amino)-3,4-dioxocyclobut-1-en-1-yl)amino)hexyl)oxy)-5-(2-fluoroacetamido)-4-hydroxytetrahydro-2H-pyran-2-carboxylic acid (9) (100 mg, 0.1 mmol) in N,N-Dimethyl formamide (2 mL) were added 2,5-dioxopyrrolidin-1-yl 3-(2,5-dioxo-2,5-dihydro-1H-pyrrol-1-yl)propanoate (10) (16.8 mg, 0.1 mmol) and triethylamine (0.04 ml, 0.32 mmol) at 0° C. The resulting reaction mixture was stirred at the same temperature for 45 min. After completion, the reaction mixture was purified by C18 prep-HPLC using acetonitrile and water (+0.05% TFA) as eluents to afford (2R,4S,5R,6R)-6-((1R,2R)-3-(2-([1,1′-biphenyl]-4-yl) acetamido)-1,2-dihydroxypropyl)-2-((6-((3,4-dioxo-2-((2-oxo-6,9,12,15-tetraoxa-3-azaheptadecan-17-yl)amino) cyclobut-1-en-1-yl)amino)hexyl)oxy)-5-(2-fluoroacetamido)-4-hydroxytetrahydro-2H-pyran-2-carboxylic acid (21) as white solid. Yield: 28 mg, 26.92%, LCMS (ESI±) m/z 977.9 [M+H]⁺. ¹H NMR (400 MHz, MeOD) δ 7.65-7.55 (m, 4H), 7.48-7.29 (m, 5H), 3.97-3.69 (m, 15H), 3.69-3.60 (m, 18H), 3.60-3.51 (m, 4H), 3.46-3.33 (m, 10H), 2.73-2.69 (m, 1H), 1.59-1.51 (m, 4H), 1.35 (dt, J=13.7, 9.0 Hz, 4H).

### (2R,4S,5R,6R)-6-((1R,2R)-3-(2-([1,1′-biphenyl]-4-yl) acetamido)-1,2-dihydroxypropyl)-2-((6-((2-((18-(2,5-dioxo-2,5-dihydro-1H-pyrrol-1-yl)-16-oxo-3,6,9,12-tetraoxa-15-azaoctadecyl)amino)-3,4-dioxocyclobut-1-en-1-yl)amino)hexyl)oxy)-5-(2-fluoroacetamido)-4-hydroxytetrahydro-2H-pyran-2-carboxylic acid (Cpd. 21M)

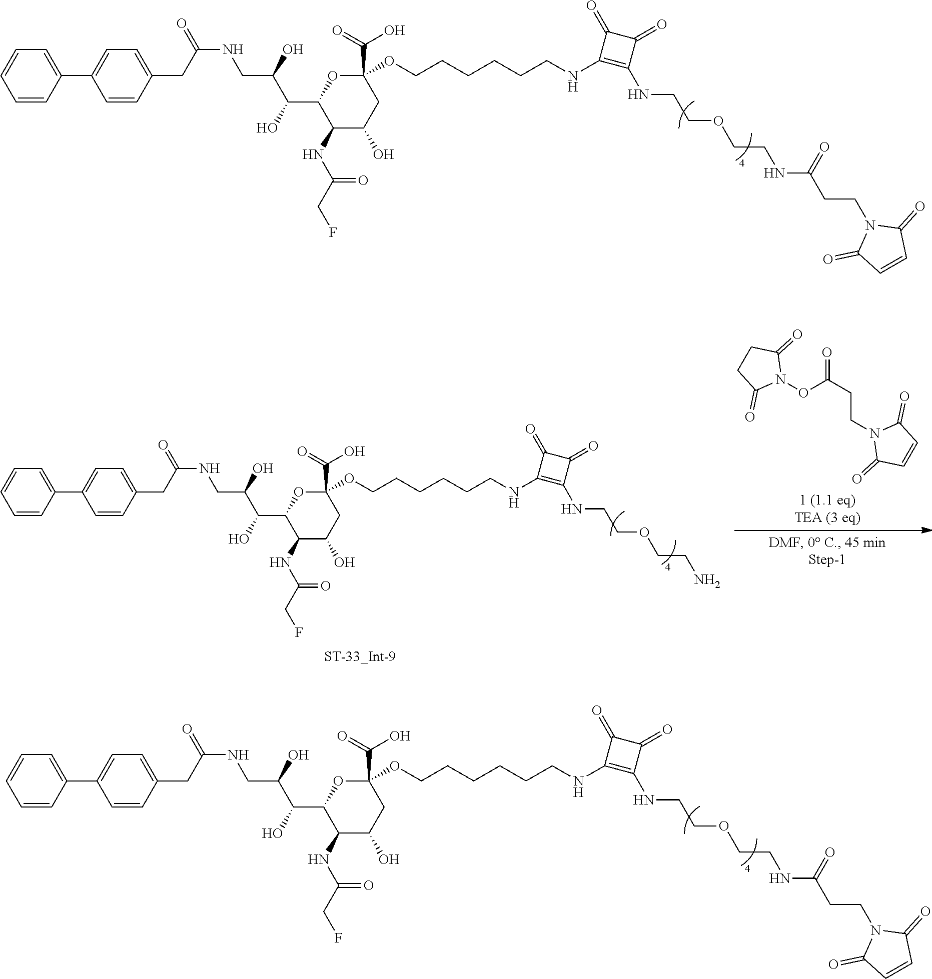

### Synthesis of (2R,4S,5R,6R)-6-((1R,2R)-3-(2-([1,1′-biphenyl]-4-yl) acetamido)-1,2-dihydroxypropyl)-2-((6-((2-((18-(2,5-dioxo-2,5-dihydro-1H-pyrrol-1-yl)-16-oxo-3,6,9,12-tetraoxa-15-azaoctadecyl)amino)-3,4-dioxocyclobut-1-en-1-yl)amino)hexyl)oxy)-5-(2-fluoroacetamido)-4-hydroxytetrahydro-2H-pyran-2-carboxylic acid (21M)

To a stirred solution of (2R,4S,5R,6R)-6-((1R,2R)-3-(2-([1,1′-biphenyl]-4-yl)acetamido)-1,2-dihydroxypropyl)-2-((6-((2-((14-amino-3,6,9,12-tetraoxatetradecyl)amino)-3,4-dioxocyclobut-1-en-1-yl)amino)hexyl)oxy)-5-(2-fluoroacetamido)-4-hydroxytetrahydro-2H-pyran-2-carboxylic acid (ST-33_Int-9) (200 mg, 0.21 mmol) in N,N-Dimethyl formamide (2 mL) were added 2,5-dioxopyrrolidin-1-yl 3-(2,5-dioxo-2,5-dihydro-1H-pyrrol-1-yl)propanoate (1) (62.65 mg, 0.23 mmol) and triethylamine (0.09 ml, 0.64 mmol) at 0° C. The resulting reaction mixture was stirred at 0° C. for 45 min. After completion, the reaction mixture was purified by C18 prep-HPLC using acetonitrile and water (+0.05%, TFA) as eluents to afford (2R,4S,5R,6R)-6-((1R,2R)-3-(2-([1,1′-biphenyl]-4-yl) acetamido)-1,2-dihydroxypropyl)-2-((6-((2-((18-(2,5-dioxo-2,5-dihydro-1H-pyrrol-1-yl)-16-oxo-3,6,9,12-tetraoxa-15-azaoctadecyl)amino)-3,4-dioxocyclobut-1-en-1-yl)amino)hexyl)oxy)-5-(2-fluoroacetamido)-4-hydroxytetrahydro-2H-pyran-2-carboxylic acid (21M) as white solid. Yield: 50 mg, 21.55%; LCMS (ESI±) m/z 1086.0 [M+H]⁺. ¹H NMR (400 MHz, MeOD) δ 8.18 (d, J=8 Hz, 1H), 7.65-7.55 (m, 4H), 7.48-7.36 (m, 4H), 7.36-7.29 (m, 1H), 6.82 (s, 2H), 4.77 (d, J=1.4 Hz, 1H), 3.97-3.69 (m, 10H), 3.69-3.54 (m, 18H), 3.51 (t, J=5.4 Hz, 2H), 3.46-3.35 (m, 3H), 3.33-3.21 (m, 1H), 2.71 (dd, J=12.7, 4.5 Hz, 1H), 2.48 (t, J=6.9 Hz, 2H), 1.80-1.69 (m, 1H), 1.56 (s, 4H), 1.35 (p, J=7.2 Hz, 4H).

### (2R,4S,5R,6R)-6-((1R,2R)-3-(2-([1,1′-biphenyl]-4-yl) acetamido)-1,2-dihydroxypropyl)-2-((6-((2-((16,32-dioxo-36-((3aR,4R,6aS)-2-oxohexahydro-1H-thieno[3,4-d] imidazol-4-yl)-3,6,9,12,19,22,25,28-octaoxa-15,31-diazahexatriacontyl)amino)-3,4-dioxocyclobut-1-en-1-yl)amino)hexyl)oxy)-5-(2-fluoroacetamido)-4-hydroxytetrahydro-2H-pyran-2-carboxylic acid (Cpd. 21B)

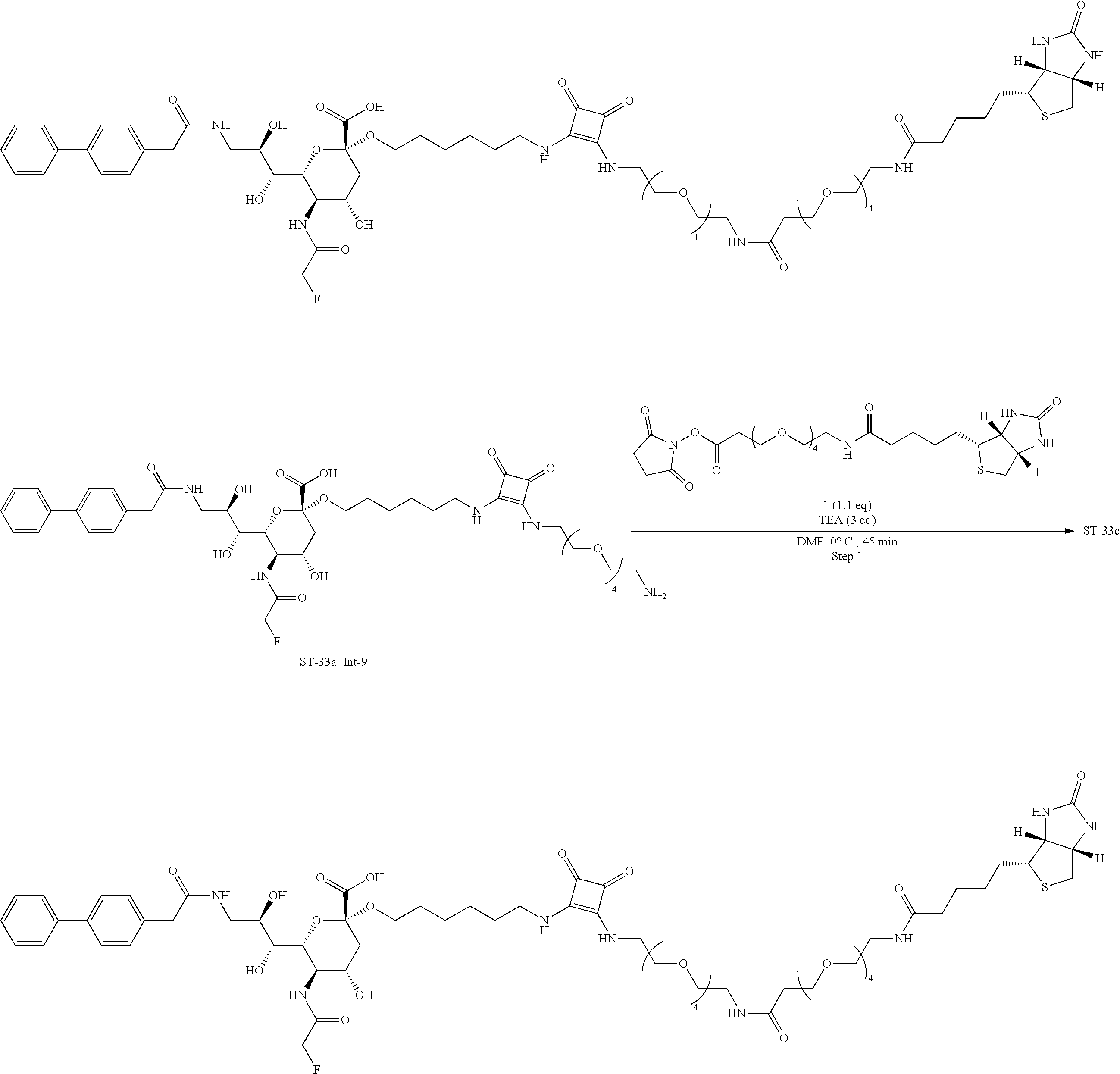

### Synthesis of (2R,4S,5R,6R)-6-((1R,2R)-3-(2-([1,1′-biphenyl]-4-yl) acetamido)-1,2-dihydroxypropyl)-2-((6-((2-((16,32-dioxo-36-((3aR,4R,6aS)-2-oxohexahydro-1H-thieno[3,4-d]imidazol-4-yl)-3,6,9,12,19,22,25,28-octaoxa-15,31-diazahexatriacontyl)amino)-3,4-dioxocyclobut-1-en-1-yl)amino)hexyl)oxy)-5-(2-fluoroacetamido)-4-hydroxytetrahydro-2H-pyran-2-carboxylic acid (21B)

To a stirred solution of (2R,4S,5R,6R)-6-((1R,2R)-3-(2-([1,1′-biphenyl]-4-yl)acetamido)-1,2-dihydroxypropyl)-2-((6-((2-((14-amino-3,6,9,12-tetraoxatetradecyl)amino)-3,4-dioxocyclobut-1-en-1-yl)amino)hexyl)oxy)-5-(2-fluoroacetamido)-4-hydroxytetrahydro-2H-pyran-2-carboxylic acid (ST-33_Int-9) (50 mg, 0.05 mmol) in N,N-Dimethyl formamide (1 mL) were added 2,5-dioxopyrrolidin-1-yl 17-oxo-21-((3aR,4R,6aS)-2-oxohexahydro-1H-thieno[3,4-d]imidazol-4-yl)-4,7,10,13-tetraoxa-16-azahenicosanoate (1) (34.62 mg, 0.05 mmol) and triethylanine (0.02 ml, 0.16 mmol) at 0° C. The resulting reaction mixture was stirred at the same temperature for 45 min. After completion, the reaction mixture was purified by C18 prep-HPLC using acetonitrile and water (+0.05%, TFA) as eluents to afford (2R,4S,5R,6R)-6-((1R,2R)-3-(2-([1,1′-biphenyl]-4-yl)acetamido)-1,2-dihydroxypropyl)-2-((6-((2-((16,32-dioxo-36-((3aR,4R,6aS)-2-oxohexahydro-1H-thieno[3,4-d]imidazol-4-yl)-3,6,9,12,19,22,25,28-octaoxa-15,31-diazahexatriacontyl)amino)-3,4-dioxocyclobut-1-en-1-yl)amino)hexyl)oxy)-5-(2-fluoroacetamido)-4-hydroxytetrahydro-2H-pyran-2-carboxylic acid (21B) as white solid. Yield: 16 mg, 21.33%; LCMS (ESI±) m/z 1408.3 [M+H]⁺. ¹H NMR (400 MHz, MeOD) δ 7.65-7.55 (m, 4H), 7.49-7.29 (m, 5H), 4.88 (s, 1H) 4.51 (dd, J=8.0, 4.8 Hz, 1H), 4.33 (dd, J=7.9, 4.4 Hz, 1H), 3.96-3.81 (m, 9H), 3.81 (s, 2H), 3.75 (t, J=6.1 Hz, 4H), 3.72-3.61 (m, 25H), 3.61-3.52 (m, 6H), 3.39 (tt, J=5.5, 2.6 Hz, 6H), 3.30-3.18 (m, 2H), 2.95 (dd, J=12.8, 5.0 Hz, 1H), 2.73 (dd, J=12.7, 4.5 Hz, 1H), 2.48 (t, J=6.2 Hz, 2H), 2.24 (t, J=7.3 Hz, 2H), 1.83-1.55 (m, 6H), 1.46 (p, J=7.9 Hz, 7H).

### (2R,4S,5R,6R)-6-((1R,2R)-3-(2-(4-chlorophenyl) acetamido)-1,2-dihydroxypropyl)-4-hydroxy-5-(2-hydroxyacetamido)-2-((6-(4-(15-oxo-2,5,8,11-tetraoxa-14-azahexadecyl)-1H-1,2,3-triazol-1-yl) hexyl)oxy) tetrahydro-2H-pyran-2-carboxylic acid (Cpd. 246)

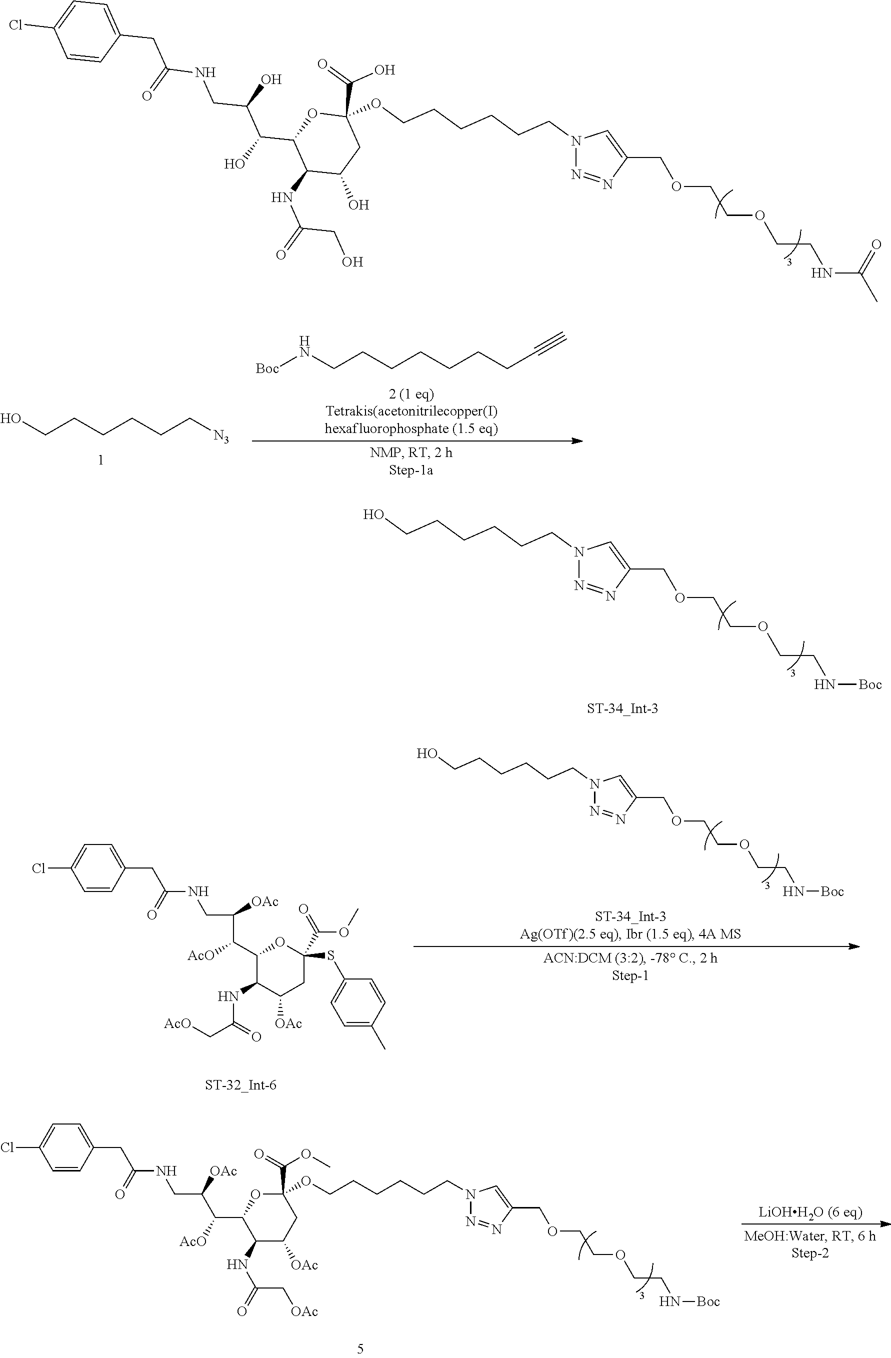

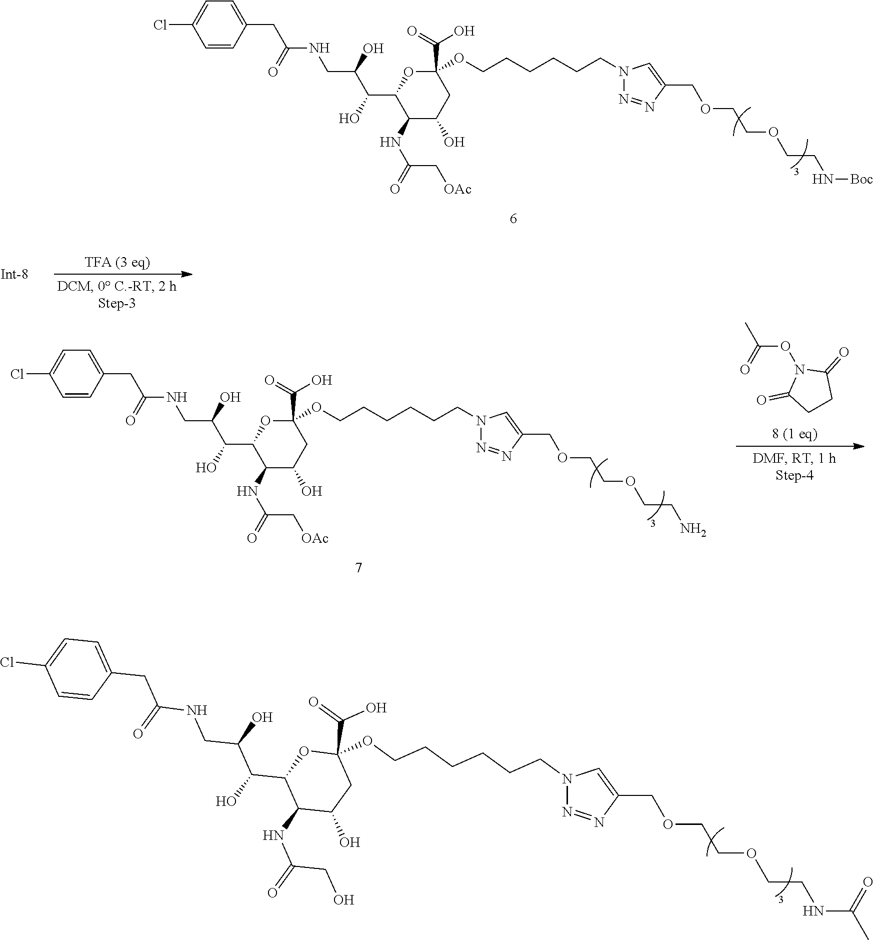

### Synthesis of tert-butyl (1-(1-(6-hydroxyhexyl)-1H-1,2,3-triazol-4-yl)-2,5,8,11-tetraoxatridecan-13-yl) carbamate (ST-34_Int-3)

To a stirred solution of 6-azidohexan-1-ol (1) (2 g, 13.9 mmol) and tert-butyl (3,6,9,12-tetraoxapentadec-14-yn-1-yl) carbamate (2) (4.6 g, 13.9 mmol) in N-methyl pyrrolidone (20 mL) was added Tetrakis(acetonitrile)copper(I)hexafluorophosphate (7.8 g, 20.9 mmol) and the reaction mixture was stirred at room temperature for 2 h. After completion, the reaction mixture was concentrated, the residue obtained was purified by C18 reverse phase column chromatography using acetonitrile and water (+0.05%, TFA) as eluents to afford tert-butyl (1-(1-(6-hydroxyhexyl)-1H-1,2,3-triazol-4-yl)-2,5,8,11-tetraoxatridecan-13-yl) carbamate (ST-34_Int-3) as off white semi solid. Yield: 2.5 g, 37.71%. ¹H NMR (400 MHz, DMSO): δ 8.09 (S, 1H), 6.76 (s, 1H), 4.50 (s, 2H), 4.33 (t, J=6.8 Hz, 2H), 3.53-3.29 (m, 16H), 3.05 (d, J=5.6 Hz, 2H), 1.79 (t, J=5.6 Hz, 2H), 1.50 (d, J=7.6 Hz, 2H), 1.39-1.30 (m, 13H).

### Synthesis of ((1R,2R)-1-((2R,3R,4S,6R)-4-acetoxy-3-(2-acetoxyacetamido)-6-((6-(4-(17,17-dimethyl-15-oxo-2,5,8,11,16-pentaoxa-14-azaoctadecyl)-1H-1,2,3-triazol-1-yl) hexyl)oxy)-6-(methoxycarbonyl) tetrahydro-2H-pyran-2-yl)-3-(2-(4-chlorophenyl) acetamido) propane-1,2-diyl diacetate (5)

To a stirred solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (4) (1.5 g, 1.96 mmol) and tert-butyl (1-(1-(6-hydroxyhexyl)-1H-1,2,3-triazol-4-yl)-2,5,8,11-tetraoxatridecan-13-yl)carbamate (3) (1.39 g, 2.94 mmol) in anhydrous acetonitrile (18 mL) and anhydrous dichloromethane (12 mL) were added 4 Å powdered molecular sieves (2 g) and Ag(OTf) (1.25 g, 4.9 mmol) under nitrogen atmosphere. The reaction mixture was stirred for 30 min., cooled to −78° C. and a solution of iodine monobromide (0.60 g, 2.94 mmol) in dichloromethane (2 ml) was dropwise added at the same temperature. The reaction mixture was continued at the same temperature for next 2 h. After completion, the reaction mixture was diluted with dichloromethane (30 ml), quenched with triethylamine (up to pH=7), filtered, the filtrate was washed with 10% aqueous of sodium thio-sulfate solution, dried over sodium sulfate, concentrated. The crude residue obtained was purified by C18 reverse phase column chromatography using acetonitrile and water (0.05%, TFA) as eluents to afford ((1R,2R)-1-((2R,3R,4S,6R)-4-acetoxy-3-(2-acetoxyacetamido)-6-((6-(4-(17,17-dimethyl-15-oxo-2,5,8,11,16-pentaoxa-14-azaoctadecyl)-1H-1,2,3-triazol-1-yl) hexyl)oxy)-6-(methoxycarbonyl) tetrahydro-2H-pyran-2-yl)-3-(2-(4-chlorophenyl) acetamido) propane-1,2-diyl diacetate (5) as yellow solid. Yield-1 g, 45%; LCMS (ESI±) m/z 1116.1 [M+H]⁺.

### Synthesis of (2R,4S,5R,6R)-6-((1R,2R)-3-(2-(4-chlorophenyl) acetamido)-1,2-dihydroxypropyl)-2-((6-(4-(17,17-dimethyl-15-oxo-2,5,8,11,16-pentaoxa-14-azaoctadecyl)-1H-1,2,3-triazol-1-yl) hexyl)oxy)-4-hydroxy-5-(2-hydroxyacetamido) tetrahydro-2H-pyran-2-carboxylic acid (6)

To a stirred solution of ((1R,2R)-1-((2R,3R,4S,6R)-4-acetoxy-3-(2-acetoxyacetamido)-6-((6-(4-(17,17-dimethyl-15-oxo-2,5,8,11,16-pentaoxa-14-azaoctadecyl)-1H-1,2,3-triazol-1-yl) hexyl)oxy)-6-(methoxycarbonyl) tetrahydro-2H-pyran-2-yl)-3-(2-(4-chlorophenyl) acetamido) propane-1,2-diyl diacetate (5) (1 g, 0.89 mmol) in tetrahydrofuran (7 mL) was added solution of lithium hydroxide monohydrate (225 mg, 5.38 mmol) in water at 0° C. and the reaction mixture was stirred at room temperature for 16 h. After completion the reaction mixture was acidified with acidic resin (Dowex 50H⁺), filtered, the filtrate was concentrated. The crude residue. The crude residue obtained was purified by C18 reverse phase column chromatography using acetonitrile and water (+0.05%, TFA) as eluents to afford (2R,4S,5R,6R)-6-((1R,2R)-3-(2-(4-chlorophenyl) acetamido)-1,2-dihydroxypropyl)-2-((6-(4-(17,17-dimethyl-15-oxo-2,5,8,11,16-pentaoxa-14-azaoctadecyl)-1H-1,2,3-triazol-1-yl) hexyl)oxy)-4-hydroxy-5-(2-hydroxyacetamido) tetrahydro-2H-pyran-2-carboxylic acid (6) as off white solid. Yield-600 mg, 71%; LCMS (ESI±) m/z 934.0 [M+H]⁺.

### Synthesis of (2R,4S,5R,6R)-2-((6-(4-(13-amino-2,5,8,11-tetraoxatridecyl)-1H-1,2,3-triazol-1-yl) hexyl)oxy)-6-((1R,2R)-3-(2-(4-chlorophenyl) acetamido)-1,2-dihydroxypropyl)-4-hydroxy-5-(2-hydroxyacetamido) tetrahydro-2H-pyran-2-carboxylic acid (7)

To a stirred solution of get (2R,4S,5R,6R)-6-((1R,2R)-3-(2-(4-chlorophenyl) acetamido)-1,2-dihydroxypropyl)-2-((6-(4-(17,17-dimethyl-15-oxo-2,5,8,11,16-pentaoxa-14-azaoctadecyl)-1H-1,2,3-triazol-1-yl) hexyl)oxy)-4-hydroxy-5-(2-hydroxyacetamido) tetrahydro-2H-pyran-2-carboxylic acid (6) (600 mg, 0.75 mmol) in anhydrous dichloromethane (6 mL), trifluoro acetic acid (0.3 mL) was added at 0° C. The reaction mixture was stirred at room temperature for 2 h. After completion, the reaction mixture was concentrated, the residue obtained was suspended in methanol and neutralized with tetra alkyl ammonium carbonate (polymer-bound). The reaction mixture was filtered and filtrate was concentrated to afford (2R,4S,5R,6R)-2-((6-(4-(13-amino-2,5,8,11-tetraoxatridecyl)-1H-1,2,3-triazol-1-yl) hexyl)oxy)-6-((1R,2R)-3-(2-(4-chlorophenyl) acetamido)-1,2-dihydroxypropyl)-4-hydroxy-5-(2-hydroxyacetamido) tetrahydro-2H-pyran-2-carboxylic acid (7) as yellow solid. Yield-500 mg, 93%; LCMS (ESI±) m/z 833.9 [M+H]⁺.

### Synthesis of (2R,4S,5R,6R)-6-((1R,2R)-3-(2-(4-chlorophenyl) acetamido)-1,2-dihydroxypr opyl)-4-hydroxy-5-(2-hydroxyacetamido)-2-((6-(4-(15-oxo-2,5,8,11-tetraoxa-14-azahexadecyl)-1H-1,2,3-triazol-1-yl) hexyl)oxy) tetrahydro-2H-pyran-2-carboxylic acid (246)

To a stirred solution of (2R,4S,5R,6R)-2-((6-(4-(13-amino-2,5,8,11-tetraoxatridecyl)-1H-1,2,3-triazol-1-yl)hexyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (7) (100 mg, 0.134 mmol) in N,N-Dimethyl formamide (1 mL) was added 2,5-dioxopyrrolidin-1-yl acetate (8) (21.06 mg, 0.134 mmol) at 0° C. The resulting reaction mixture was stirred at room temperature for 1 h. After completion, the reaction mixture was purified by C18 prep-HPLC using acetonitrile and water (+0.05%, TFA) as eluents to afford (2R,4S,5R,6R)-6-((1R,2R)-3-(2-(4-chlorophenyl) acetamido)-1,2-dihydroxypr opyl)-4-hydroxy-5-(2-hydroxyacetamido)-2-((6-(4-(15-oxo-2,5,8,11-tetraoxa-14-azahexadecyl)-1H-1,2,3-triazol-1-yl) hexyl)oxy) tetrahydro-2H-pyran-2-carboxylic acid (246) as white solid. Yield: 27 mg, 25%; LCMS (ESI±) m/z 873.7 [M−H]⁻. ¹H NMR (400 MHz, MeOD): δ 8.01 (s, 1H), 7.92 (d, J=7.2 Hz, 1H), 7.31 (s, 4H) 4.66 (s, 2H), 4.42 (t, J=6.8 Hz, 2H), 4.04 (s, 2H), 3.89-3.63 (m, 18H), 3.47-3.37 (m, 5H), 2.87 (t, J=2.4 Hz, 5H), 2.73 (dd, J=12.7, 4.1 Hz, 1H), 1.74 (t, J=11.9 Hz, 1H), 1.96-1.90 (m, 5H), 1.64-1.54 (m, 4H), 1.51-1.39 (m, 4H).

### (2R,4S,5R,6R)-6-((1R,2R)-3-(2-(4-chlorophenyl) acetamido)-1,2-dihydroxypropyl)-2-((6-(4-(17-(2,5-dioxo-2,5-dihydro-1H-pyrrol-1-yl)-15-oxo-2,5,8,11-tetraoxa-14-azaheptadecyl)-1H-1,2,3-triazol-1-yl) hexyl)oxy)-4-hydroxy-5-(2-hydroxyacetamido) tetrahydro-2H-pyran-2-carboxylic acid (Cpd. 246M)

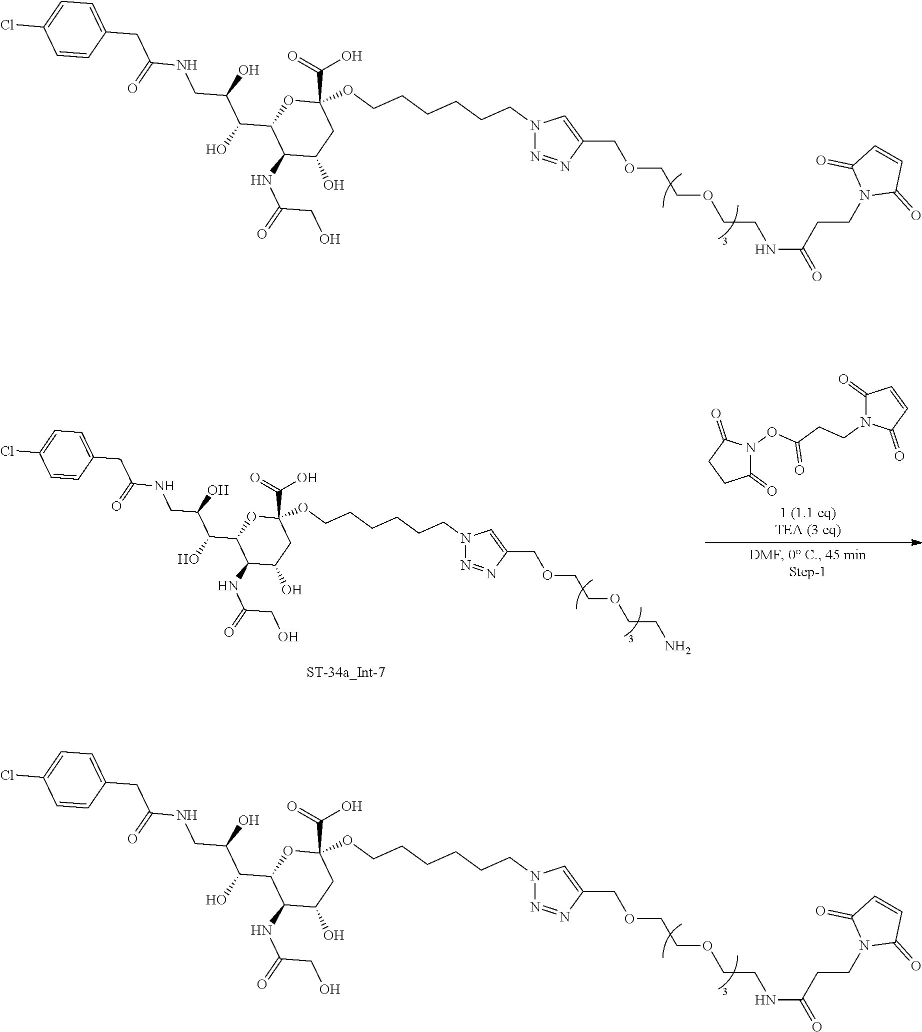

### Synthesis of (2R,4S,5R,6R)-6-((1R,2R)-3-(2-(4-chlorophenyl) acetamido)-1,2-dihydroxypropyl)-2-((6-(4-(17-(2,5-dioxo-2,5-dihydro-1H-pyrrol-1-yl)-15-oxo-2,5,8,11-tetraoxa-14-azaheptadecyl)-1H-1,2,3-triazol-1-yl) hexyl)oxy)-4-hydroxy-5-(2-hydroxyacetamido) tetrahydro-2H-pyran-2-carboxylic acid (246M)

To a stirred solution of (2R,4S,5R,6R)-2-((6-(4-(13-amino-2,5,8,11-tetraoxatridecyl)-1H-1,2,3-triazol-1-yl)hexyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (7) (200 mg, 0.240 mmol) in N,N-Dimethyl formamide (2 mL) were added 2,5-dioxopyrrolidin-1-yl 3-(2,5-dioxo-2,5-dihydro-1H-pyrrol-1-yl)propanoate (8) (63.89 mg, 0.240 mmol) and triethylamine (0.1 mL, 0.72 mmol) at 0° C. The resulting reaction mixture was stirred at 0° C. for 45 min. After completion, the reaction mixture was purified by C18 prep-HPLC using acetonitrile and water (+0.05%, TFA) as eluents to afford (2R,4S,5R,6R)-6-((1R,2R)-3-(2-(4-chlorophenyl) acetamido)-1,2-dihydroxypropyl)-2-((6-(4-(17-(2,5-dioxo-2,5-dihydro-1H-pyrrol-1-yl)-15-oxo-2,5,8,11-tetraoxa-14-azaheptadecyl)-1H-1,2,3-triazol-1-yl) hexyl)oxy)-4-hydroxy-5-(2-hydroxyacetamido) tetrahydro-2H-pyran-2-carboxylic acid (246M) as white solid. Yield: 56 mg, 23%; LCMS (ESI±) m/z 982.7 [M−H]⁻. ¹H NMR (400 MHz, MeOD): δ 8.01 (s, 1H), 7.93 (d, J=7.8 Hz, 1H), 7.31 (s, 4H), 6.83 (s, 2H), 4.66 (s, 2H), 4.43 (t, J=7.2, 2H), 4.04 (s, 2H), 3.89-3.68 (m, 20H), 3.65-3.60 (m, 5H), 3.47-3.37 (m, 2H), 3.19 (dt, J=6.9, 3.4 Hz, 1H), 2.71 (dd, J=12.7, 4.5 Hz, 1H) 2.49 (t, J=7.2 Hz, 2H), 1.94 (t, J=6.8 Hz, 2H), 1.76 (t, J=12.0 Hz, 1H), 1.51-1.31 (m, 7H).

### (2R,4S,5R,6R)-6-((1R,2R)-3-(2-(4-chlorophenyl)acetamido)-1,2-dihydroxypropyl)-2-((6-(4-(15,31-dioxo-35-((3aR,4R,6aS)-2-oxohexahydro-1H-thieno[3,4-d]imidazol-4-yl)-2,5,8,11,18,21,24,27-octaoxa-14,30-diazapentatriacontyl)-1H-1,2,3-triazol-1-yl)hexyl)oxy)-4-hydroxy-5-(2-hydroxyacetamido)tetrahydro-2H-pyran-2-carboxylic acid (Cpd. 246B)

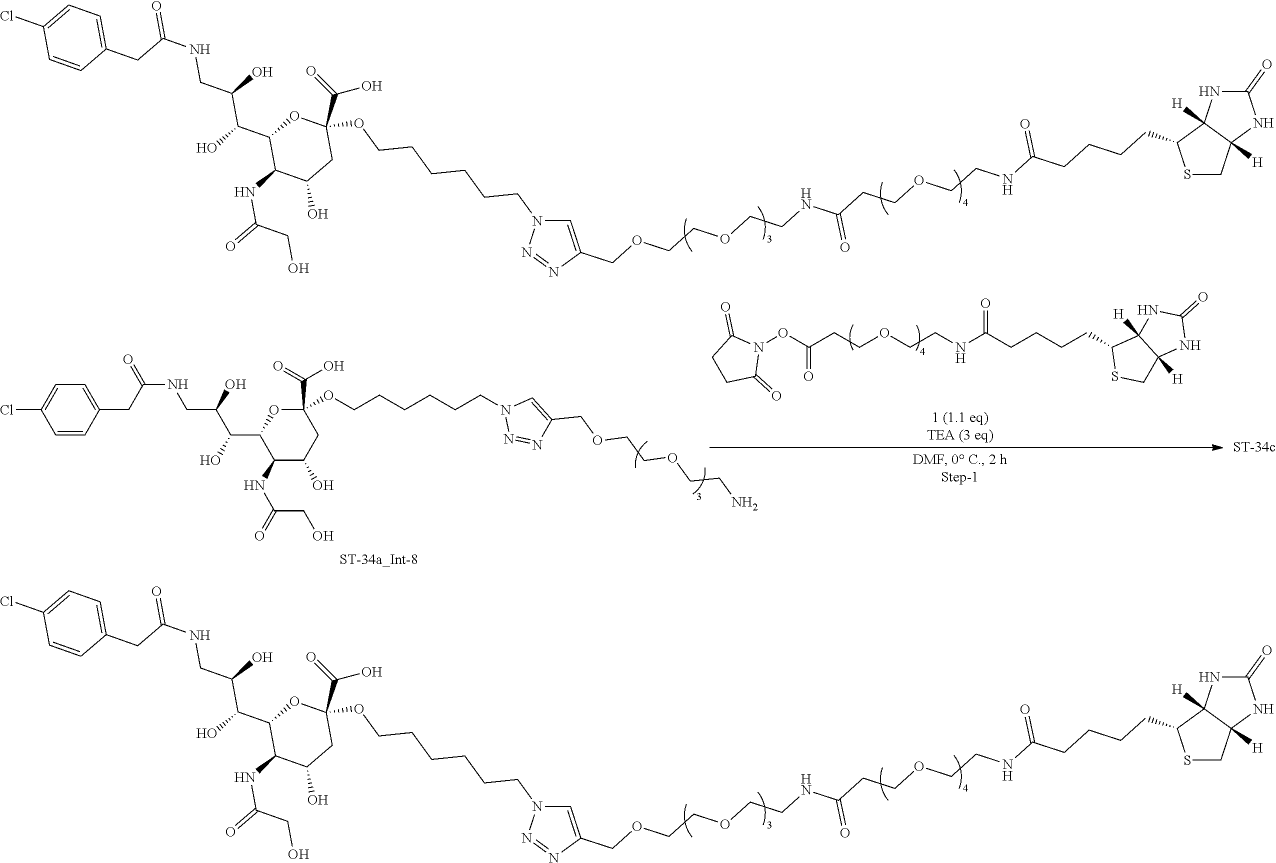

### Synthesis of (2R,4S,5R,6R)-6-((1R,2R)-3-(2-(4-chlorophenyl)acetamido)-1,2-dihydroxypropyl)-2-((6-(4-(15,31-dioxo-35-((3aR,4R, 6aS)-2-oxohexahydro-1H-thieno[3,4-d]imidazol-4-yl)-2, 5,8,11,18, 21, 24, 27-octaoxa-14,30-diazapentatriacontyl)-1H-1, 2, 3-triazol-1-yl)hexyl)oxy)-4-hydroxy-5-(2-hydroxyacetamido)tetrahydro-2H-pyran-2-carboxylic acid (246B)

To a stirred solution of (2R,4S,5R,6R)-2-((6-(4-(13-amino-2,5,8,11-tetraoxatridecyl)-1H-1,2,3-triazol-1-yl)hexyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (246_Int-8) (50 mg, 0.06 mmol) in anhydrous N,N-dimethyl formamide (1 mL) were added 2,5-dioxopyrrolidin-1-yl 17-oxo-21-((3 aR,4R,6aS)-2-oxohexahydro-1H-thieno[3,4-d]imidazol-4-yl)-4,7,10,13-tetraoxa-16-azahenicosanoate (1) (38 mg, 0.06 mmol) and triethylamine (0.02 ml, 0.16 mmol) at 0° C. The resulting reaction mixture was stirred at 0° C. for 2 h. After completion, the reaction mixture was purified by prep-HPLC purification by using acetonitrile and water (+0.05%, TFA) as eluents to afford (2R,4S,5R,6R)-6-((1R,2R)-3-(2-(4-chlorophenyl)acetamido)-1,2-dihydroxypropyl)-2-((6-(4-(15,31-dioxo-35-((3aR,4R,6aS)-2-oxohexahydro-1H-thieno[3,4-d]imidazol-4-yl)-2,5,8,11,18,21,24,27-octaoxa-14,30-diazapentatriacontyl)-1H-1,2,3-triazol-1-yl)hexyl)oxy)-4-hydroxy-5-(2-hydroxyacetamido)tetrahydro-2H-pyran-2-carboxylic acid (246B) as white solid, Yield: 15 mg, 20%; LCMS (ESI+) m/z 1307.8 [M+H]⁺. ¹H NMR (400 MHz, MeOD) δ 8.00 (s, 1H), 7.30 (s, 4H), 4.65 (s, 2H), 4.30-4.32 (m, 4H), 4.04 (s, 2H), 3.88-3.74 (m, 7H), 3.72-3.65 (m, 24H), 3.63-3.54 (m, 6H), 3.42-3.32 (m, 6H), 3.24-3.21 (m, 2H), 2.74-2.70 (m, 3H), 2.47 (t, J=6.0 Hz, 2H), 2.25-2.22 (m, 2H), 1.91 (t, J=7.0 Hz, 2H), 1.73-1.53 (m, 7H), 1.47-1.31 (m, 7H).

### (2R,4S,5R,6R)-6-((1R,2R)-3-(2-(4-chlorophenyl) acetamido)-1,2-dihydroxypropyl)-2-(2-(2-(2-((3,4-dioxo-2-((2-oxo-6,9,12,15-tetraoxa-3-azaheptadecan-17-yl)amino) cyclobut-1-en-1-yl)amino) ethoxy) ethoxy) ethoxy)-4-hydroxy-5-(2-hydroxyacetamido) tetrahydro-2H-pyran-2-carboxylic acid (Cpd. 247)

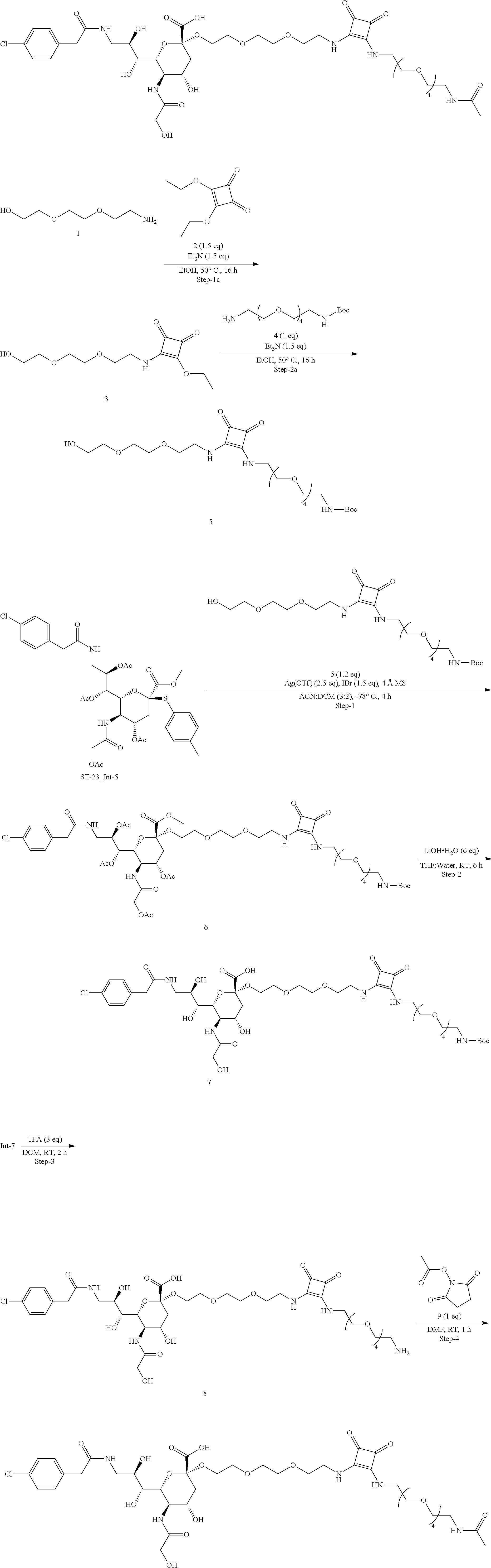

### Synthesis of 3-ethoxy-4-((2-(2-(2-hydroxyethoxy)ethoxy)ethyl)amino) cyclobut-3-ene-1,2-dione (3)

To a stirred solution of 2-(2-(2-aminoethoxy) ethoxy) ethan-1-ol (4 g, 26.84 mmol) and 3,4-diethoxycyclobut-3-ene-1,2-dione (2) (6.84 g, 40.26 mmol) in Ethanol (40 mL) was added triethylamine (5.82 ml, 40.26 mmol) and the reaction mixture was stirred at 50° C. for 16 h. After completion, the reaction mixture was concentrated and purified by neutral alumina column chromatography using methanol and dichloromethane as eluents to afford 3-ethoxy-4-((2-(2-(2-hydroxyethoxy)ethoxy)ethyl)amino) cyclobut-3-ene-1,2-dione (3) pale yellow gummy material. Yield: 5 g, 17.80%; LCMS (ESI±) m/z 274.3 [M+H]⁺.

### Synthesis of tert-butyl (14-((2-((2-(2-(2-hydroxyethoxy)ethoxy)ethyl)amino)-3,4-dioxocyclobut-1-en-1-yl)amino)-3,6,9,12-tetraoxatetradecyl) carbamate (5)

To a stirred solution of 3-ethoxy-4-((2-(2-(2-hydroxyethoxy)ethoxy)ethyl)amino)cyclobut-3-ene-1,2-dione (3) (1.3 g, 4.76 mmol) and tert-butyl (14-amino-3,6,9,12-tetraoxatetradecyl)carbamate (4) (1.6 g, 4.76 mmol) in Ethanol (13 mL) was added triethylamine (1.18 ml, 8.22 mmol) at room temperature. The resulting reaction mixture was stirred at 60° C. for 16 h. After completion, the reaction mixture was purified by C-18 reverse phase column chromatography using acetonitrile and water (+0.05%, TFA) as eluents to afford tert-butyl (14-((2-((2-(2-(2-hydroxyethoxy)ethoxy)ethyl)amino)-3,4-dioxocyclobut-1-en-1-yl) amino)-3,6,9,12-tetraoxatetradecyl) carbamate (5) as yellow solid. Yield: 0.96 g, 35.82%; LCMS (ESI±) m/z 562.6 [M−H]⁻.

### Synthesis of (1R,2R)-1-((2R,3R,4S,6R)-4-acetoxy-3-(2-acetoxyacetamido)-6-(2-(2-(2-((2-((2,2-dimethyl-4-oxo-3,8,11,14,17-pentaoxa-5-azanonadecan-19-yl)amino)-3,4-dioxocyclobut-1-en-1-yl)amino) ethoxy) ethoxy) ethoxy)-6-(methoxycarbonyl) tetrahydro-2H-pyran-2-yl)-3-(2-(4-chlorophenyl) acetamido) propane-1,2-diyl diacetate (6)

To a stirred solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (ST-23_Int-5) (1.5 g, 1.9 mmol) and tert-butyl (14-((2-((2-(2-(2-hydroxyethoxy)ethoxy)ethyl)amino)-3,4-dioxocyclobut-1-en-1-yl)amino)-3,6,9,12-tetraoxatetradecyl)carbamate (5) (1.65 g, 2.94 mmol) in anhydrous acetonitrile (18 mL) and anhydrous dichloromethane (12 mL) were added 4 Å powdered molecular sieves (5 g) and Ag(OTf) (1.25 g, 4.9 mmol) under nitrogen atmosphere. The reaction mixture was stirred for 30 min then cooled to −78° C. and added solution of iodine monobromide (0.6 g, 2.9 mmol) in dichloromethane (4 ml). The reaction mixture was continued at the same temperature for next 5 h. After completion, the reaction mixture was diluted with dichloromethane (15 ml), quenched with triethylamine (up to pH=7), filtered, filtrate was washed with 10% aqueous Sodium thio-sulfate solution, dried over sodium sulfate, concentrated. The crude residue obtained was purified by C-18 reverse phase column chromatography using acetonitrile and water (+0.05%, TFA) as eluents to afford the desired product (1R,2R)-1-((2R,3R,4S,6R)-4-acetoxy-3-(2-acetoxyacetamido)-6-(2-(2-(2-((2-((2,2-dimethyl-4-oxo-3,8,11,14,17-pentaoxa-5-azanonadecan-19-yl)amino)-3,4-dioxocyclobut-1-en-1-yl)amino) ethoxy) ethoxy) ethoxy)-6-(methoxycarbonyl) tetrahydro-2H-pyran-2-yl)-3-(2-(4-chlorophenyl) acetamido) propane-1,2-diyl diacetate (6) as yellow solid. Yield: 1 g, 38.02%; LCMS (ESI±) m/z 1205.2[M+H]⁺.

### Synthesis of (2R,4S,5R,6R)-6-((1R,2R)-3-(2-(4-chlorophenyl) acetamido)-1,2-dihydroxypropyl)-2-(2-(2-(2-((2-((2,2-dimethyl-4-oxo-3,8,11,14,17-pentaoxa-5-azanonadecan-19-yl)amino)-3,4-dioxocyclobut-1-en-1-yl)amino) ethoxy) ethoxy) ethoxy)-4-hydroxy-5-(2-hydroxyacetamido) tetrahydro-2H-pyran-2-carboxylic acid (7)

To a stirred solution of (1R,2R)-1-((2R,3R,4S,6R)-3-acetamido-4-acetoxy-6-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-6-(methoxycarbonyl) tetrahydro-2H-pyran-2-yl)-3-(2-(4-chlorophenyl) acetamido) propane-1,2-diyl diacetate (6) (1 g, 0.83 mmol) in methanol (9 mL) was added lithium hydroxide monohydrate (0.2 mg, 4.98 mmol) dissolved in water (1 mL) at 0° C. and the reaction mixture was stirred at room temperature for 16 h. After completion the reaction mixture was acidified with acidic resin (Dowex 50H⁺) to pH˜6 and the suspension was filtered, filtrate was concentrated. The crude residue obtained was purified by C-18 reverse phase column chromatography using acetonitrile and water (+0.05%, TFA) as eluents to afford the desired product (2R,4S,5R,6R)-6-((1R,2R)-3-(2-(4-chlorophenyl) acetamido)-1,2-dihydroxypropyl)-2-(2-(2-(2-((2-((2,2-dimethyl-4-oxo-3,8,11,14,17-pentaoxa-5-azanonadecan-19-yl)amino)-3,4-dioxocyclobut-1-en-1-yl)amino) ethoxy) ethoxy) ethoxy)-4-hydroxy-5-(2-hydroxyacetamido) tetrahydro-2H-pyran-2-carboxylic acid (7) as off white solid. Yield: 0.33 g, 38.86%; LCMS (ESI±) m/z 1022.9 [M+H]⁺.

### Synthesis of (2R,4S,5R,6R)-2-(2-(2-(2-((2-((14-amino-3,6,9,12-tetraoxatetradecyl)amino)-3,4-dioxocyclobut-1-en-1-yl)amino) ethoxy) ethoxy) ethoxy)-6-((1R,2R)-3-(2-(4-chlorophenyl) acetamido)-1,2-dihydroxypropyl)-4-hydroxy-5-(2-hydroxyacetamido) tetrahydro-2H-pyran-2-carboxylic acid (8)

To a stirred solution of (2R,4S,5R,6R)-6-((1R,2R)-3-(2-(4-chlorophenyl) acetamido)-1,2-dihydroxypropyl)-2-(2-(2-(2-((2-((2,2-dimethyl-4-oxo-3,8,11,14,17-pentaoxa-5-azanonadecan-19-yl)amino)-3,4-dioxocyclobut-1-en-1-yl)amino) ethoxy) ethoxy) ethoxy)-4-hydroxy-5-(2-hydroxyacetamido) tetrahydro-2H-pyran-2-carboxylic acid (7) (0.33 g, 0.32 mmol) in anhydrous dichloromethane (4.5 mL) was added trifluoro acetic acid (1 mL) at 0° C. Then the reaction mixture was stirred at room temperature for 2 h. After completion, the reaction mixture was concentrated. The crude residue obtained was suspended in methanol and neutralized with tetra alkyl ammonium carbonate (polymer-bound resin). The reaction mixture was filtered and filtrate was concentrated to afford the desired product (2R,4S,5R,6R)-5-acetamido-2-((6-((2-((14-amino-3,6,9,12-tetraoxatetradecyl)amino)-3,4-dioxocyclobut-1-en-1-yl)amino)hexyl)oxy)-6-((1R,2R)-3-(2-(4-chlorophenyl) acetamido)-1,2-dihydroxypropyl)-4-hydroxytetrahydro-2H-pyran-2-carboxylic acid (8) as yellow solid. Yield: 250 mg, 62.03%; LCMS (ESI±) m/z 920.9 [M−H]⁻.

### Synthesis of (2R,4S,5R,6R)-6-((1R,2R)-3-(2-(4-chlorophenyl) acetamido)-1,2-dihydroxypropyl)-2-(2-(2-(2-((3,4-dioxo-2-((2-oxo-6,9,12,15-tetraoxa-3-azaheptadecan-17-yl)amino) cyclobut-1-en-1-yl)amino) ethoxy) ethoxy) ethoxy)-4-hydroxy-5-(2-hydroxyacetamido) tetrahydro-2H-pyran-2-carboxylic acid (247)

To a stirred solution of (2R,4S,5R,6R)-2-(2-(2-(2-((2-((14-amino-3,6,9,12-tetraoxatetradecyl)amino)-3,4-dioxocyclobut-1-en-1-yl)amino)ethoxy)ethoxy)ethoxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (8) (120 mg, 0.13 mmol) in N,N-Dimethyl formamide (1.2 mL) was added 2,5-dioxopyrrolidin-1-yl acetate (9) (20.4 mg, 0.13 mmol) at 0° C. The resulting reaction mixture was stirred at room temperature for 1 h. After completion, the reaction mixture was purified by prep-HPLC using acetonitrile and water (+0.05%, TFA) as eluents to afford the desired product (2R,4S,5R,6R)-6-((1R,2R)-3-(2-(4-chlorophenyl) acetamido)-1,2-dihydroxypropyl)-2-(2-(2-(2-((3,4-dioxo-2-((2-oxo-6,9,12,15-tetraoxa-3-azaheptadecan-17-yl)amino) cyclobut-1-en-1-yl)amino) ethoxy) ethoxy) ethoxy)-4-hydroxy-5-(2-hydroxyacetamido) tetrahydro-2H-pyran-2-carboxylic acid (247) as white solid. Yield: 10 mg, 8%; LCMS (ESI±) m/z 964.6 [M+H]⁺. ¹H NMR (400 MHz, MeOD) δ 7.32 (s, 4H), 4.05 (s, 2H), 3.91 (s, 2H), 3.91-3.82 (m, 5H), 3.71-3.62 (m, 24H), 3.56 (d, J=6.6 Hz, 5H), 3.44-3.35 (m, 3H), 3.25 (dd, J=13.9, 8.0 Hz, 1H), 2.74 (dd, J=12.3, 4.0 Hz, 1H), 1.99 (s, 3H), 1.79 (t, J=10.8 Hz, 2H).

### (2R,4S,5R,6R)-6-((1R,2R)-3-(2-(4-chlorophenyl) acetamido)-1,2-dihydroxypropyl)-2-(2-(2-(2-((2-((18-(2,5-dioxo-2,5-dihydro-1H-pyrrol-1-yl)-16-oxo-3,6,9,12-tetraoxa-15-azaoctadecyl)amino)-3,4-dioxocyclobut-1-en-1-yl)amino) ethoxy) ethoxy) ethoxy)-4-hydroxy-5-(2-hydroxyacetamido) tetrahydro-2H-pyran-2-carboxylic acid (Cpd. 247M)

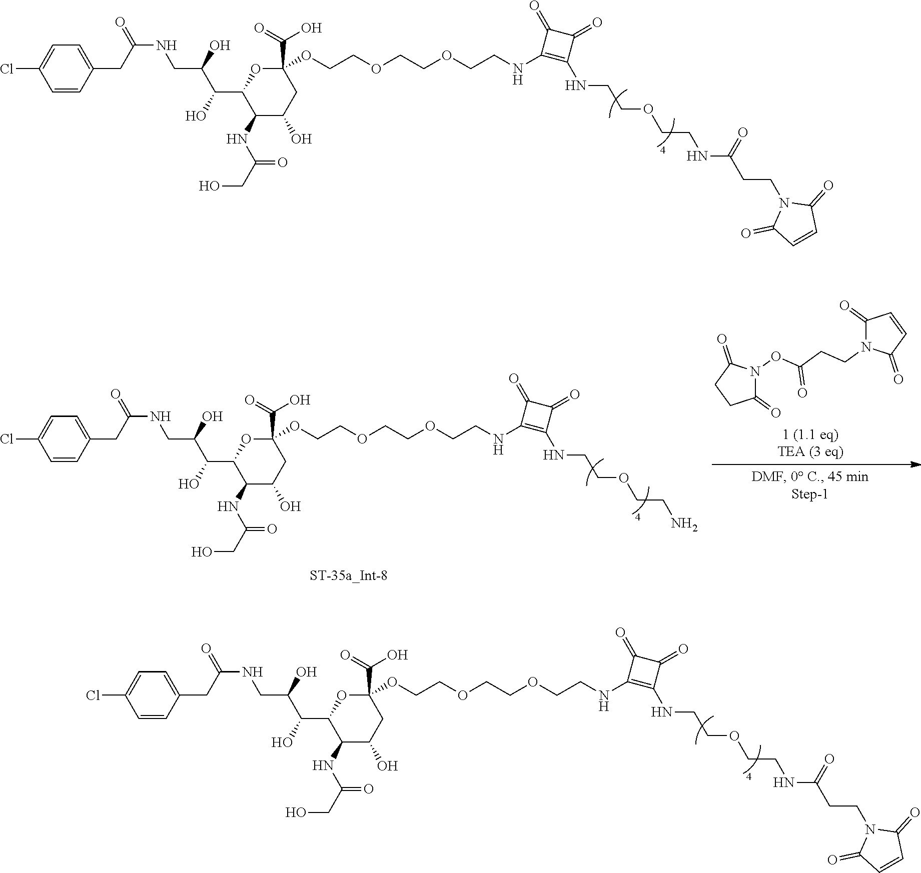

### Synthesis of (2R,4S,5R,6R)-6-((1R,2R)-3-(2-(4-chlorophenyl) acetamido)-1,2-dihydroxypropyl)-2-(2-(2-(2-((2-((18-(2,5-dioxo-2,5-dihydro-1H-pyrrol-1-yl)-16-oxo-3,6,9,12-tetraoxa-15-azaoctadecyl)amino)-3,4-dioxocyclobut-1-en-1-yl)amino) ethoxy) ethoxy) ethoxy)-4-hydroxy-5-(2-hydroxyacetamido) tetrahydro-2H-pyran-2-carboxylic acid (247M)

To a stirred solution of (2R,4S,5R,6R)-2-(2-(2-(2-((2-((14-amino-3,6,9,12-tetraoxatetradecyl)amino)-3,4-dioxocyclobut-1-en-1-yl)amino)ethoxy)ethoxy)ethoxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (247_Int-8) (188 mg, 0.2 mmol) in N,N-Dimethyl formamide (2 mL) were added 2,5-dioxopyrrolidin-1-yl 3-(2,5-dioxo-2,5-dihydro-1H-pyrrol-1-yl)propanoate (1) (50 mg, 0.21 mmol) and triethylamine (0.08 ml, 0.61 mmol) at 0° C. The resulting reaction mixture was stirred at 0° C. for 45 min. After completion, the reaction mixture was purified by prep-HPLC using acetonitrile and water (+0.05%, TFA) as eluents to afford the desired product (2R,4S,5R,6R)-6-((1R,2R)-3-(2-(4-chlorophenyl) acetamido)-1,2-dihydroxypropyl)-2-(2-(2-(2-((2-((18-(2,5-dioxo-2,5-dihydro-1H-pyrrol-1-yl)-16-oxo-3,6,9,12-tetraoxa-15-azaoctadecyl)amino)-3,4-dioxocyclobut-1-en-1-yl)amino) ethoxy) ethoxy) ethoxy)-4-hydroxy-5-(2-hydroxyacetamido) tetrahydro-2H-pyran-2-carboxylic acid (247M). Yield: 30 mg, 12.93%; LCMS (ESI) m/z 1074.4 [M+H]⁺. ¹H NMR (400 MHz, MeOD) δ 7.31 (s, 4H), 6.83 (s, 2H), 4.05 (s, 2H), 3.95-3.87 (m, 2H), 3.80 (dd, J=12.5, 5.4 Hz, 8H), 3.65 (qt, J=7.1, 3.1 Hz, 28H), 3.60-3.48 (m, 4H), 3.41-3.32 (m, 1H), 3.21 (dd, J=13.5, 8.0 Hz, 1H), 2.81 (dd, J=12.8 Hz, 1H), 2.49 (t, J=6.9 Hz, 2H), 1.68 (t, J=11.8 Hz, 1H).

### (2R,4S,5R,6R)-6-((1R,2R)-3-(2-(4-chlorophenyl) acetamido)-1,2-dihydroxypropyl)-2-(2-(2-(2-((2-((16,32-dioxo-36-((3aR,4R,6aS)-2-oxohexahydro-1H-thieno[3,4-d] imidazol-4-yl)-3,6,9,12,19,22,25,28-octaoxa-15,31-diazahexatriacontyl)amino)-3,4-dioxocyclobut-1-en-1-yl)amino) ethoxy) ethoxy) ethoxy)-4-hydroxy-5-(2-hydroxyacetamido) tetrahydro-2H-pyran-2-carboxylic acid (Cpd. 247B)

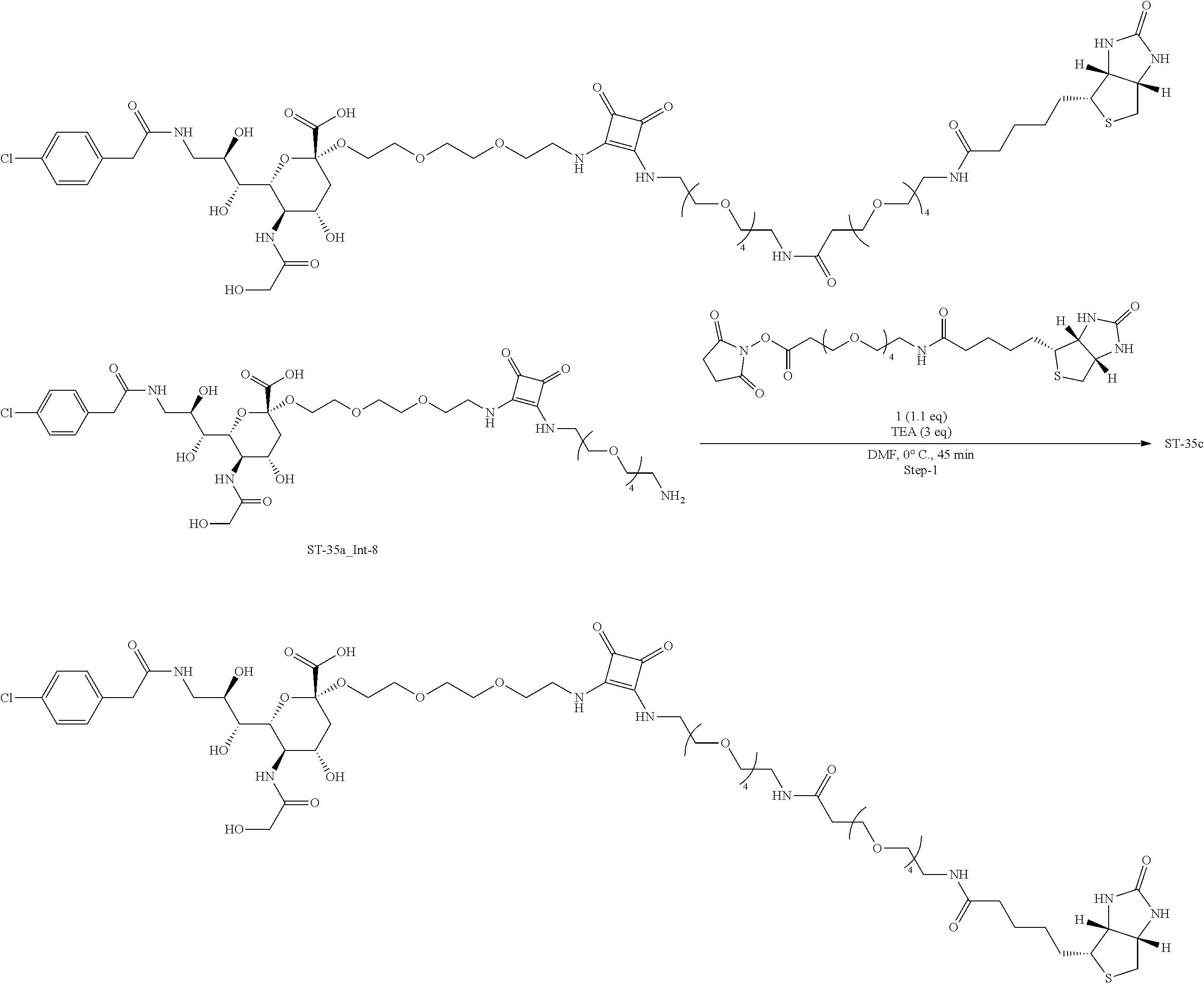

### Synthesis of (2R,4S,5R,6R)-6-((1R,2R)-3-(2-(4-chlorophenyl) acetamido)-1,2-dihydroxypropyl)-2-(2-(2-(2-((2-((16,32-dioxo-36-((3aR,4R,6aS)-2-oxohexahydro-1H-thieno[3,4-d] imidazol-4-yl)-3,6,9,12,19,22,25,28-octaoxa-15,31-diazahexatriacontyl)amino)-3,4-dioxocyclobut-1-en-1-yl)amino) ethoxy) ethoxy) ethoxy)-4-hydroxy-5-(2-hydroxyacetamido) tetrahydro-2H-pyran-2-carboxylic acid (247B)

To a stirred solution of (2R,4S,5R,6R)-2-(2-(2-(2-((2-((14-amino-3,6,9,12-tetraoxatetradecyl)amino)-3,4-dioxocyclobut-1-en-1-yl)amino)ethoxy)ethoxy)ethoxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (8) (50 mg, 0.05 mmol) in N,N-Dimethyl formamide (2 mL) were added 2,5-dioxopyrrolidin-1-yl 17-oxo-21-((3aR,4R,6aS)-2-oxohexahydro-1H-thieno[3,4-d]imidazol-4-yl)-4,7,10,13-tetraoxa-16-azahenicosanoate (1) (35 mg, 0.06 mmol) and triethylamine (0.02 ml, 0.16 mmol) at 0° C. The resulting reaction mixture was stirred at 0° C. for 45 min. After completion, the reaction mixture was purified by prep-HPLC using acetonitrile and water (+0.05%, TFA) to afford the desired product (2R,4S,5R,6R)-6-((1R,2R)-3-(2-(4-chlorophenyl) acetamido)-1,2-dihydroxypropyl)-2-(2-(2-(2-((2-((16,32-dioxo-36-((3aR,4R,6aS)-2-oxohexahydro-1H-thieno[3,4-d] imidazol-4-yl)-3,6,9,12,19,22,25,28-octaoxa-15,31-diazahexatriacontyl)amino)-3,4-dioxocyclobut-1-en-1-yl) amino)ethoxy)ethoxy)ethoxy)-4-hydroxy-5-(2-hydroxyacetamido)tetrahydro-2H-pyran-2-carboxylic acid (247B). Yield: 25 mg, 33.06%; LCMS (ESI±) m/z 1394.2 [M−H]⁻. ¹H NMR (400 MHz, MeOD) δ 7.31 (s, 4H), 4.51 (dd, J=8.0, 4.8 Hz, 1H), 4.33 (dd, J=7.9, 4.4 Hz, 1H), 4.04 (s, 2H), 3.96-3.81 (m, 7H), 3.75 (t, J=6.1 Hz, 3H), 3.72-3.61 (m, 36H), 3.61-3.52 (m, 6H), 3.39 (tt, J=5.5, 2.6 Hz, 5H), 3.30-3.18 (m, 2H), 2.95 (dd, J=12.8, 5.0 Hz, 1H), 2.80-2.69 (m, 2H), 2.48 (t, J=6.2 Hz, 2H), 2.24 (t, J=7.3 Hz, 2H), 1.83-1.55 (m, 6H), 1.46 (p, J=7.9 Hz, 2H).

### (2R,4S,5R,6R)-5-acetamido-6-((1R,2R)-3-(2-(4-chlorophenyl)acetamido)-1,2-dihydroxypropyl)-2-((6-((3,4-dioxo-2-((2-oxo-6,9,12,15-tetraoxa-3-azaheptadecan-17-yl) amino) cyclobut-1-en-1-yl)amino)hexyl)oxy)-4-hydroxytetrahydro-2H-pyran-2-carboxylic acid (Cpd. 248)

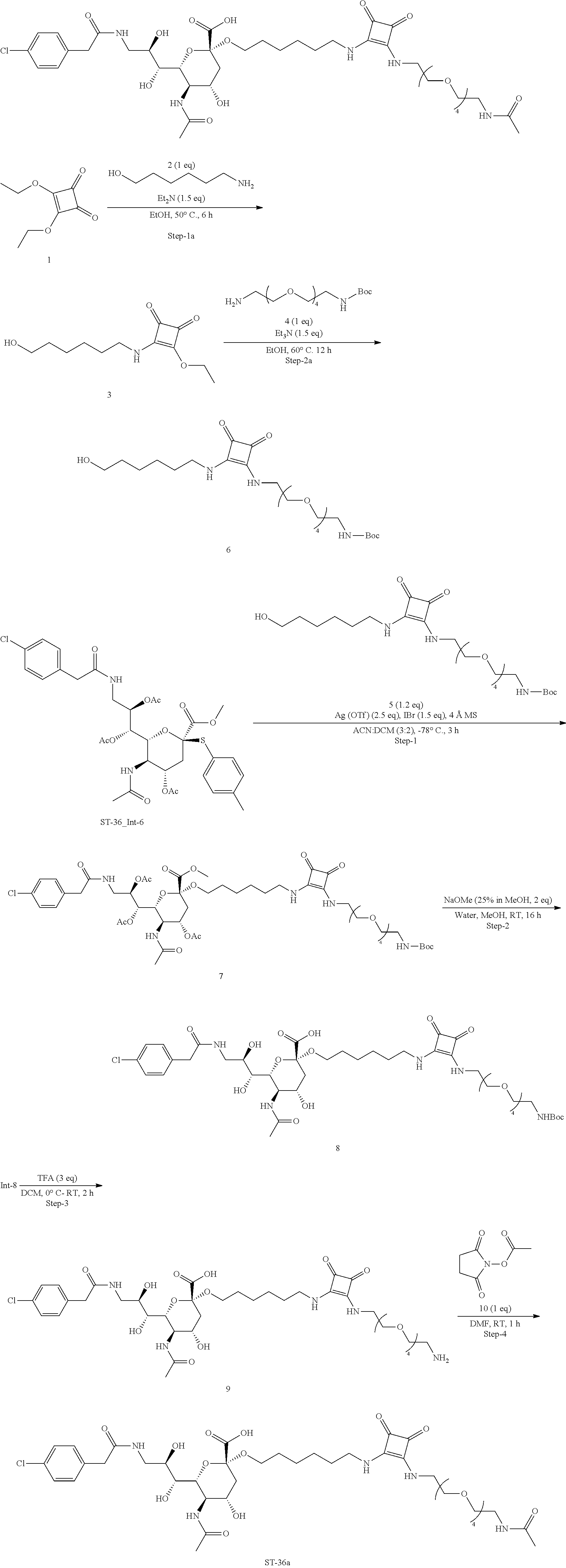

### Synthesis of 3-ethoxy-4-((6-hydroxyhexyl)amino) cyclobut-3-ene-1,2-dione (3)

To a stirred solution of 3,4-diethoxycyclobut-3-ene-1,2-dione (1) (5 g, 29.41 mmol) in Ethanol (50 mL) was added 6-aminohexan-1-ol (2) (1.72 g, 14.7 mmol) followed by addition of triethylamine (6.36 ml, 44.11 mmol). The resulting reaction mixture was stirred at 50° C. for next 6 h. After completion, the reaction mixture was concentrated. The crude residue obtained was purified by silica gel column chromatography using ethyl acetate and hexane as eluents to afford 3-ethoxy-4-((6-hydroxyhexyl)amino) cyclobut-3-ene-1,2-dione (3) as yellow semi solid. Yield: 5 g, 70.62%; LCMS (ESI±) m/z 242.2 [M+H]⁺.

### Synthesis of tert-butyl (14-((2-((6-hydroxyhexyl)amino)-3,4-dioxocyclobut-1-en-1-yl)amino)-3,6,9,12-tetraoxatetradecyl) carbamate (5)

To a stirred solution of 3-ethoxy-4-((6-hydroxyhexyl)amino)cyclobut-3-ene-1,2-dione (3) (5 g, 20.72 mmol) in Ethanol (50 mL) were added triethylamine (4.49 ml, 31.12 mmol) and tert-butyl (14-amino-3,6,9,12-tetraoxatetradecyl)carbamate (4) (6.97 g, 20.72 mmol) and the resulting reaction mixture was stirred at 60° C. for 12 h. After completion, the reaction mixture was concentrated. The crude residue obtained was purified by silica gel column chromatography, using ethyl acetate and hexane as eluents to afford tert-butyl (14-((2-((6-hydroxyhexyl)amino)-3,4-dioxocyclobut-1-en-1-yl)amino)-3,6,9,12-tetraoxatetradecyl) carbamate (5) as white solid. Yield: 3 g, 27.27%; LCMS (ESI±) m/z 532.4 [M+H]⁺.

### Synthesis of (1R,2R)-1-((2R,3R,4S,6R)-3-acetamido-4-acetoxy-6-((6-((2-((2,2-dimethyl-4-oxo-3,8,11,14,17-pentaoxa-5-azanonadecan-19-yl)amino)-3,4-dioxocyclobut-1-en-1-yl)amino) hexyl)oxy)-6-(methoxycarbonyl) tetrahydro-2H-pyran-2-yl)-3-(2-(4-chlorophenyl) acetamido) propane-1,2-diyl diacetate (6)

To a stirred solution of (1R,2R)-1-((2R,3R,4S,6R)-3-acetamido-4-acetoxy-6-(methoxycarbonyl)-6-(p-tolylthio)tetrahydro-2H-pyran-2-yl)-3-(2-(4-chlorophenyl)acetamido)propane-1,2-diyl diacetate (ST-36_Int-5) (2 g, 2.82 mmol) and tert-butyl (14-((2-((6-hydroxyhexyl)amino)-3,4-dioxocyclobut-1-en-1-yl)amino)-3,6,9,12-tetraoxatetradecyl)carbamate (5) (1.8 g, 3.39 mmol) in anhydrous dichloromethane (12 mL) and anhydrous acetonitrile (24 mL) were added Silver (+1) trifluoro methane sulfonate Ag(OTf) (1.8 g, 7.07 mmol) and activated 4 Å powdered molecular sieves (5 g) and the reaction mixture was stirred at room temperature for 1 h under nitrogen atmosphere. Then reaction mixture was cooled to −78° C. and added iodine monobromide (0.87 g, 4.24 mmol, 1.5 eq.) dissolved in dichloromethane (4 ml) dropwise. The reaction mixture was stirred at the same temperature for 2 h. After completion, the reaction mixture was quenched with triethylamine (5 mL) and warmed to room temperature. Then the reaction mixture was filtered, filtrate was washed by saturated aqueous sodium thiosulfate solution, dried over sodium sulfate, filtered, concentrated. The crude residue was purified by C-18 reverse phase column chromatography using water and acetonitrile (+0.05%, TFA) as eluents to afford the desired product (1R,2R)-1-((2R,3R,4S,6R)-3-acetamido-4-acetoxy-6-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-6-(methoxycarbonyl) tetrahydro-2H-pyran-2-yl)-3-(2-(4-chlorophenyl) acetamido) propane-1,2-diyl diacetate (6) as yellow solid. Yield: 1.4 g, 44.44%; LCMS (ESI±) m/z 1112.8 [M−H]⁻.

### Synthesis of (2R,4S,5R,6R)-5-acetamido-6-((1R,2R)-3-(2-(4-chlorophenyl) acetamido)-1,2-dihydroxypropyl)-2-((6-((2-((2,2-dimethyl-4-oxo-3,8,11,14,17-pentaoxa-5-azanonadecan-19-yl) amino)-3,4-dioxocyclobut-1-en-1-yl)amino)hexyl)oxy)-4-hydroxytetrahydro-2H-pyran-2-carboxylic acid (7)

To a stirred solution of (1R,2R)-1-((2R,3R,4S,6R)-3-acetamido-4-acetoxy-6-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-6-(methoxycarbonyl)tetrahydro-2H-pyran-2-yl)-3-(2-(4-chlorophenyl)acetamido)propane-1,2-diyl diacetate (6) (900 mg, 0.80 mmol) in methanol (7 mL) and water (3 mL) was added 25% sodium methoxide in methanol (0.5 mL, 5.80 mmol) at 0° C. and the reaction mixture was stirred at room temperature for 16 h. After completion the reaction mixture was treated with acidic resin (Dowex 50, H⁺) to make the pH˜6 and the suspension was filtered, filtrate was concentrated to afford the desired product (2R,4S,5R,6R)-5-acetamido-6-((1R,2R)-3-(2-(4-chlorophenyl) acetamido)-1,2-dihydroxypropyl)-2-((6-((2-((2,2-dimethyl-4-oxo-3,8,11,14,17-pentaoxa-5-azanonadecan-19-yl)amino)-3,4-dioxocyclobut-1-en-1-yl)amino) hexyl)oxy)-4-hydroxytetrahydro-2H-pyran-2-carboxylic acid (7) as off white solid. Yield: 1 g (Crude); LCMS (ESI±) m/z 972.9 [M−H]⁻.

### Synthesis of (2R,4S,5R,6R)-5-acetamido-2-((6-((2-((14-amino-3,6,9,12-tetraoxatetradecyl) amino)-3,4-dioxocyclobut-1-en-1-yl)amino)hexyl)oxy)-6-((1R,2R)-3-(2-(4-chlorophenyl) acetamido)-1,2-dihydroxypropyl)-4-hydroxytetrahydro-2H-pyran-2-carboxylic acid (8)

To a stirred solution of (2R,4S,5R,6R)-5-acetamido-6-((1R,2R)-3-(2-(4-chlorophenyl) acetamido)-1,2-dihydroxypropyl)-2-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-4-hydroxytetrahydro-2H-pyran-2-carboxylic acid (7) (0.45 g, 0.46 mmol) in anhydrous dichloromethane (4.5 mL) was added trifluoro acetic acid (1 mL) at 0° C. The reaction mixture was stirred at room temperature for 2 h. After completion, the reaction mixture was concentrated. The crude residue obtained was dissolved in methanol and neutralized with tetra alkyl ammonium carbonate, the polymer-bound resin. Then the reaction mixture was filtered and filtrate was concentrated to afford the desired product (2R,4S,5R,6R)-5-acetamido-2-((6-((2-((14-amino-3,6,9,12-tetraoxatetradecyl)amino)-3,4-dioxocyclobut-1-en-1-yl)amino)hexyl)oxy)-6-((1R,2R)-3-(2-(4-chlorophenyl) acetamido)-1,2-dihydroxypropyl)-4-hydroxytetrahydro-2H-pyran-2-carboxylic acid (8) as yellow solid. Yield: 250 mg, 62.03%; LCMS (ESI±) m/z 876.6 [M+H]⁺.

### Synthesis of (2R,4S,5R,6R)-5-acetamido-6-((1R,2R)-3-(2-(4-chlorophenyl) acetamido)-1,2-dihydroxypropyl)-2-((6-((3,4-dioxo-2-((2-oxo-6,9,12,15-tetraoxa-3-azaheptadecan-17-yl) amino) cyclobut-1-en-1-yl)amino)hexyl)oxy)-4-hydroxytetrahydro-2H-pyran-2-carboxylic acid (248)

To a stirred solution of (2R,4S,5R,6R)-5-acetamido-2-((6-((2-((14-amino-3,6,9,12-tetraoxatetradecyl)amino)-3,4-dioxocyclobut-1-en-1-yl)amino)hexyl)oxy)-6-((1R,2R)-3-(2-(4-chlorophenyl)acetamido)-1,2-dihydroxypropyl)-4-hydroxytetrahydro-2H-pyran-2-carboxylic acid (8) (100 mg, 0.12 mmol) in N,N-dimethyl formamide (1 mL) was added 2,5-dioxopyrrolidin-1-yl acetate (10) (17.8 mg, 0.14 mmol) at 0° C. The resulting reaction mixture was stirred at room temperature for 1 h. After completion, the reaction mixture was concentrated and purified by prep-HPLC using acetonitrile and water (+0.05%, TFA) to afford the desired product (2R,4S,5R,6R)-5-acetamido-6-((1R,2R)-3-(2-(4-chlorophenyl) acetamido)-1,2-dihydroxypropyl)-2-((6-((3,4-dioxo-2-((2-oxo-6,9,12,15-tetraoxa-3-azaheptadecan-17-yl)amino) cyclobut-1-en-1-yl)amino) hexyl)oxy)-4-hydroxytetrahydro-2H-pyran-2-carboxylic acid (248) as white solid. Yield: 10 mg, 9.61%; LCMS (ESI±) m/z 918.7 [M+H]⁺. ¹H NMR (400 MHz, MeOD): δ 7.32 (s, 4H), 3.90 (td, J=8.3, 3.1 Hz, 1H), 3.87-3.76 (m, 5H), 3.76-3.64 (m, 24H), 3.76-3.64 (m, 24H), 3.61-3.56 (m, 6H), 3.38-3.33 (m, 4H), 3.29-3.22 (m, 1H), 2.73 (dd, J=13.5, 8.4 Hz, 1H), 1.98 (s, 6H), 1.72 (t, J=11.8 Hz, 1H), 1.60 (q, J=7.5 Hz, 4H), 1.40 (q, J=7.5 Hz, 4H).

### (2R,4S,5R,6R)-5-acetamido-6-((1R,2R)-3-(2-(4-chlorophenyl) acetamido)-1,2-dihydroxypropyl)-2-((6-((2-((18-(2,5-dioxo-2,5-dihydro-1H-pyrrol-1-yl)-16-oxo-3,6,9,12-tetraoxa-15-azaoctadecyl)amino)-3,4-dioxocyclobut-1-en-1-yl)amino)hexyl)oxy)-4-hydroxytetrahydro-2H-pyran-2-carboxylic acid (Cpd. 247M)

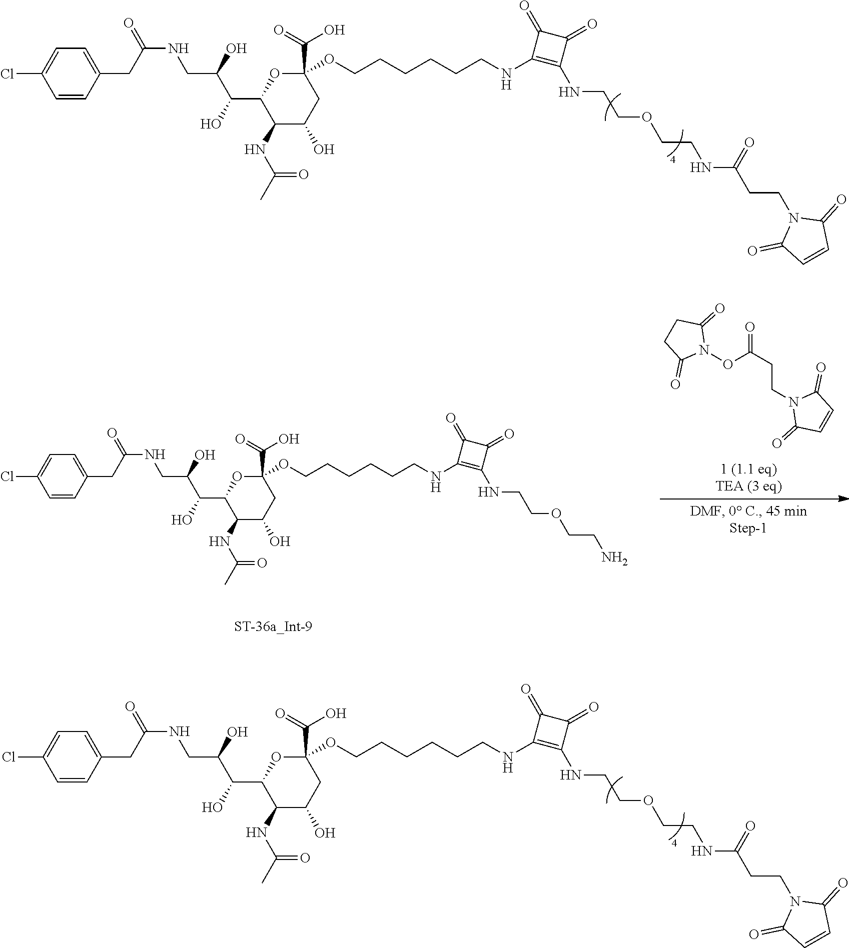

### Synthesis of (2R,4S,5R,6R)-5-acetamido-2-((6-((2-((14-amino-3,6,9,12-tetraoxatetradecyl) amino)-3,4-dioxocyclobut-1-en-1-yl)amino)hexyl)oxy)-6-((1R,2R)-3-(2-(4-chlorophenyl) acetamido)-1,2-dihydroxypropyl)-4-hydroxytetrahydro-2H-pyran-2-carboxylic acid (247M)

To a stirred solution of (2R,4S,5R,6R)-5-acetamido-2-((6-((2-((14-amino-3,6,9,12-tetraoxatetradecyl)amino)-3,4-dioxocyclobut-1-en-1-yl)amino)hexyl)oxy)-6-((1R,2R)-3-(2-(4-chlorophenyl)acetamido)-1,2-dihydroxypropyl)-4-hydroxytetrahydro-2H-pyran-2-carboxylic acid (248_Int-9) (200 mg, 0.22 mmol) in N,N-dimethyl formamide (2 mL) were added 2,5-dioxopyrrolidin-1-yl 3-(2,5-dioxo-2,5-dihydro-1H-pyrrol-1-yl)propanoate (1) (60.8 mg, 0.22 mmol) and triethylamine (0.1 mL, 0.684 mmol) at 0° C. The resulting reaction mixture was stirred at the same temperature for 45 min. After completion, the reaction mixture was concentrated and purified by prep-HPLC using acetonitrile and water (+0.05%, TFA) as eluents to afford the desired product (2R,4S,5R,6R)-5-acetamido-6-((1R,2R)-3-(2-(4-chlorophenyl) acetamido)-1,2-dihydroxypropyl)-2-((6-((2-((18-(2,5-dioxo-2,5-dihydro-1H-pyrrol-1-yl)-16-oxo-3,6,9,12-tetraoxa-15-azaoctadecyl)amino)-3,4-dioxocyclobut-1-en-1-yl)amino)hexyl)oxy)-4-hydroxytetrahydro-2H-pyran-2-carboxylic acid (247M) as white solid. Yield: 42 mg, 17.94%; LCMS (ESI±) m/z 1026.80 [M+H]⁺. ¹HNMR (400 MHz, MeOD) δ 7.31 (s, 4H), 6.83 (s, 2H), 3.89-3.68 (m, 8H), 3.66 (d, J=5.0 Hz, 19H), 3.62-3.48 (m, 5H), 3.47-3.34 (m, 2H), 3.33-3.19 (m, 1H), 2.78-2.67 (m, 1H), 2.49 (m, 2H), 2.00 (s, 3H), 1.72 (t, J=11.9 Hz, 1H), 1.59 (dt, J=12.9, 5.6 Hz, 4H), 1.40 (m, 4H).

### (2R,4S,5R,6R)-5-acetamido-6-((1R,2R)-3-(2-(4-chlorophenyl) acetamido)-1,2-dihydroxypropyl)-4-hydroxy-2-((5,21,37-trioxo-1-((3aR,4R,6aS)-2-oxohexahydro-1H-thieno[3,4-d] imidazol-4-yl)-9,12,15,18,25,28,31,34-octaoxa-6,22,38-triazatritetracontan-43-yl) oxy) tetrahydro-2H-pyran-2-carboxylic acid (Cpd. 249B)

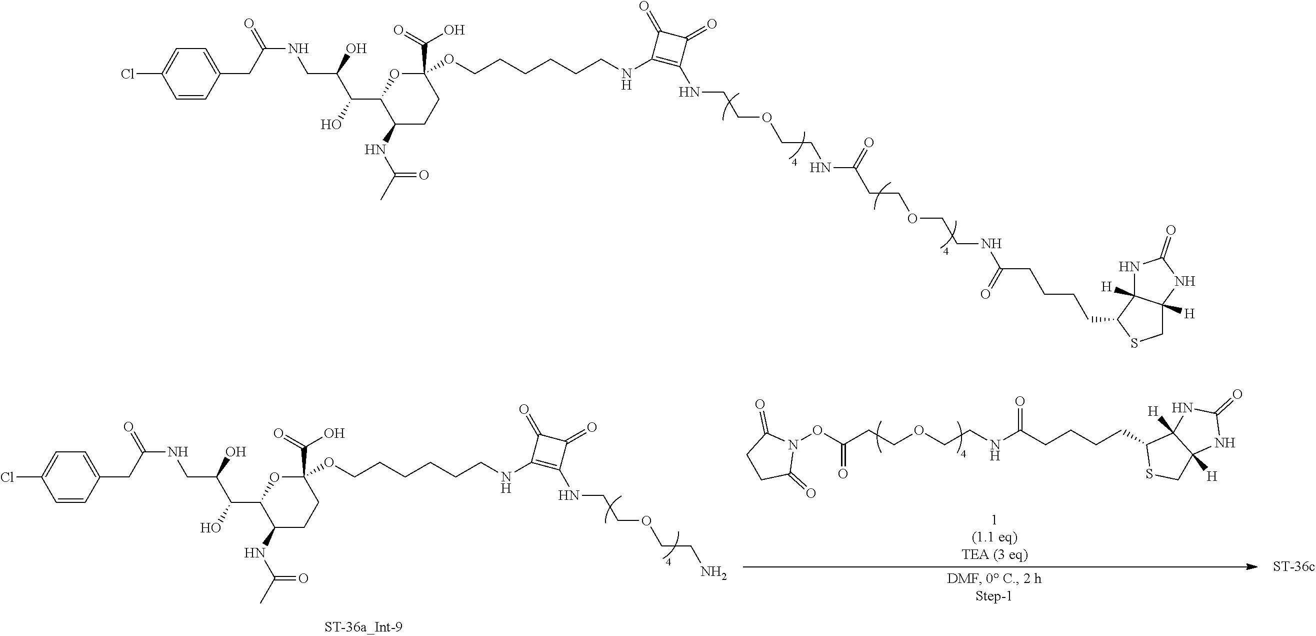

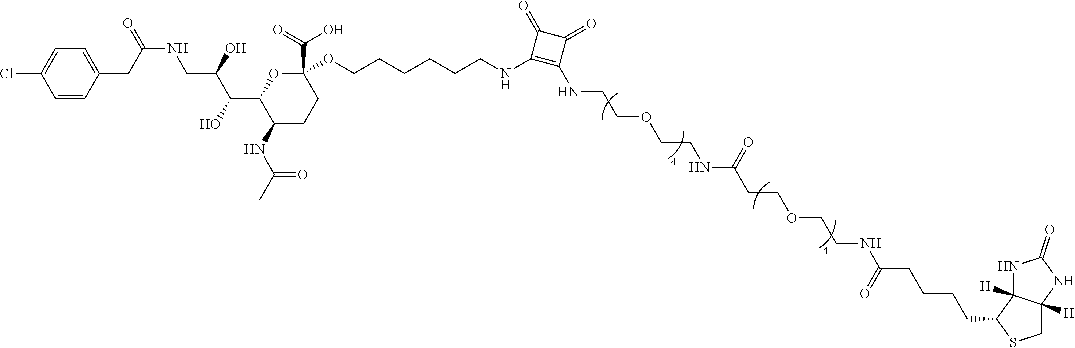

### Synthesis of (2R,4S,5R,6R)-5-acetamido-6-((1R,2R)-3-(2-(4-chlorophenyl) acetamido)-1,2-dihydroxypropyl)-2-((6-((2-((16,32-dioxo-36-((3aR,4R,6aS)-2-oxohexahydro-1H-thieno[3,4-d]imidazol-4-yl)-3,6,9,12,19,22,25,28-octaoxa-15,31-diazahexatriacontyl)amino)-3,4-dioxocyclobut-1-en-1-yl)amino)hexyl)oxy)-4-hydroxytetrahydro-2H-pyran-2-carboxylic acid (247B)

To a stirred solution (2R,4S,5R,6R)-5-acetamido-2-((6-((2-((14-amino-3,6,9,12-tetraoxatetradecyl)amino)-3,4-dioxocyclobut-1-en-1-yl)amino)hexyl)oxy)-6-((1R,2R)-3-(2-(4-chlorophenyl)acetamido)-1,2-dihydroxypropyl)-4-hydroxytetrahydro-2H-pyran-2-carboxylic acid (248_Int-9) (50 mg, 0.05 mmol) in N—N-Dimethylformamide (0.5 mL) were added 2,5-dioxopyrrolidin-1-yl 17-oxo-21-((3aR,4R,6aS)-2-oxohexahydro-1H-thieno[3,4-d]imidazol-4-yl)-4,7,10,13-tetraoxa-16-azahenicosanoate (1) (33.6 mg, 0.05 mmol) and triethylamine (0.2 ml, 0.1 mmol) at 0° C. The resulting reaction mixture was stirred at the same temperature for 45 min. After completion, the reaction mixture was concentrated and purified by prep-HPLC using acetonitrile and water as eluents to afford the desired product (2R,4S,5R,6R)-5-acetamido-6-((1R,2R)-3-(2-(4-chlorophenyl) acetamido)-1,2-dihydroxypropyl)-2-((6-((2-((16,32-dioxo-36-((3aR,4R,6aS)-2-oxohexahydro-1H-thieno[3,4-d] imidazol-4-yl)-3,6,9,12,19,22,25,28-octaoxa-15,31-diazahexatriacontyl)amino)-3,4-dioxocyclobut-1-en-1-yl)amino)hexyl)oxy)-4-hydroxytetrahydro-2H-pyran-2-carboxylic acid (247B) as white solid. Yield: 40 mg, 51.94%; LCMS (ESI) m/z 1348.86 [M+H]⁺. ¹H NMR (401 MHz, MeOD) δ 7.31 (s, 4H), 4.55-4.47 (m, 1H), 4.33 (dd, J=7.9, 4.5 Hz, 1H), 3.90 (td, J=8.3, 3.1 Hz, 1H), 3.81-3.71 (m, 2H), 3.67 (d, J=4.3 Hz, 5H), 3.66-3.60 (m, 29H), 3.60-3.53 (m, 7H), 3.48-3.33 (m, 8H), 2.95 (dd, J=12.7, 5.0 Hz, 1H), 2.73 (d, J=12.5 Hz, 2H), 2.48 (t, J=6.2 Hz, 2H), 2.24 (t, J=7.4 Hz, 2H), 2.00 (s, 3H), 1.72 (ddd, J=32.3, 14.2, 6.8 Hz, 2H), 1.67-1.57 (m, 9H), 1.63-1.56 (m, 3H), 1.47 (q, J=7.5 Hz, 2H), 1.40 (s, 5H), 1.31 (s, 1H).

### (2R,4S,5R,6R)-5-acetamido-6-((1R,2R)-3-(2-(4-chlorophenyl) acetamido)-1,2-dihydroxypropyl)-2-((2,18-dioxo-6,9,12,15-tetraoxa-3,19-diazatetracosan-24-yl) oxy)-4-hydroxytetrahydro-2H-pyran-2-carboxylic acid (Cpd. 249)

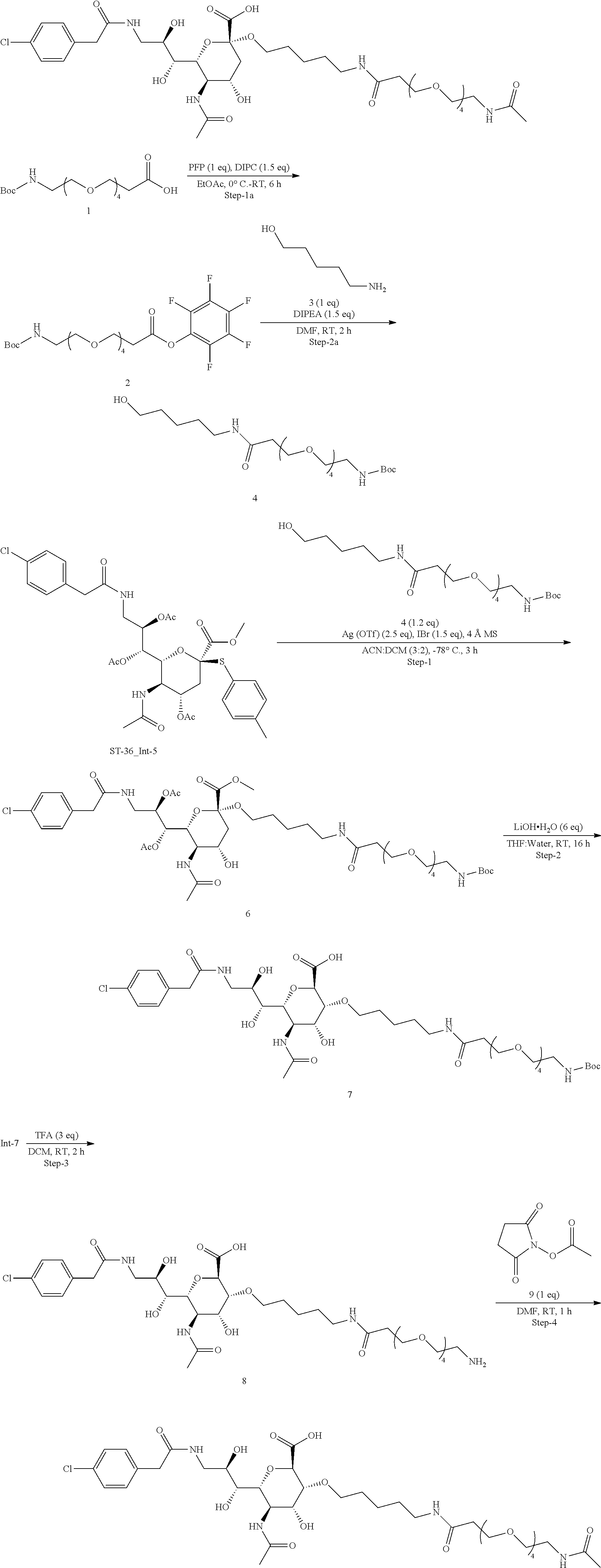

### Synthesis of perfluorophenyl 2,2-dimethyl-4-oxo-3,8,11,14,17-pentaoxa-5-azaicosan-20-oate (2)

To a stirred solution of 2,2-dimethyl-4-oxo-3,8,11,14,17-pentaoxa-5-azaicosan-20-oic acid (1) (5 g, 13.6 mmol) and penta-fluoro phenol (2.5 g, 13.6 mmol) in dry ethyl acetate (50 mL) was added di-isopropyl carbodimide (2.58 g, 20.4 mmol) dropwise at 0° C. The reaction mixture was stirred at room temperature for next 6 h. After completion, the reaction mixture was filtered, filtrate was concentrated. The crude residue obtained was purified by silica gel column chromatography, eluting with 20% EA in hexane, to afford perfluorophenyl 2,2-dimethyl-4-oxo-3,8,11,14,17-pentaoxa-5-azaicosan-20-oate (2) as off white semi solid. Yield: 5 g, 69.44%; LCMS (ESI±) m/z 533.65 [M+2]⁺.

### Synthesis of tert-butyl (21-hydroxy-15-oxo-3,6,9,12-tetraoxa-16-azahenicosyl) carbamate (4)

To a stirred solution of perfluorophenyl 2,2-dimethyl-4-oxo-3,8,11,14,17-pentaoxa-5-azaicosan-20-oate (2) (4 g, 7.52 mmol) N, N-dimethyl formamide were added 5-aminopentan-1-ol (3) (3.11 g, 6.77 mmol) and triethylamine (3.25 ml, 22.58 mmol) at 0° C. The resulting reaction mixture was stirred at room temperature for 2 h. After completion, the reaction mixture was purified by C-18 reverse phase column chromatography using acetonitrile and water (+0.05%, TFA) as eluents to afford tert-butyl (21-hydroxy-15-oxo-3,6,9,12-tetraoxa-16-azahenicosyl) carbamate (4) as white solid. Yield: 3.4 g, quant. ¹H NMR (400 MHz, DMSO): δ 7.79 (t, J=5.2 Hz, 1H), 6.75 (t, J=5.6, 1H), 5.6 (s, 1H), 3.59 (t, J=6.4 Hz, 2H), 3.50-3.45 (m, 12H), 3.39-3.31 (m, 4H), 3.17-2.99 (m, 4H), 2.29 (t, J=6.4 Hz, 2H), 1.44-1.36 (m, 13H), 1.20-1.14 (m, 3H) Synthesis of (1R,2R)-1-((2R,3R,4S,6R)-3-acetamido-4-acetoxy-6-((2,2-dimethyl-4,20-dioxo-3,8,11,14,17-pentaoxa-5,21-diazahexacosan-26-yl) oxy)-6-(methoxycarbonyl) tetrahydro-2H-pyran-2-yl)-3-(2-(4-chlorophenyl) acetamido) propane-1,2-diyl diacetate (6)

To a stirred solution of (1R,2R)-1-((2R,3R,4S,6R)-3-acetamido-4-acetoxy-6-(methoxycarbonyl)-6-(p-tolylthio)tetrahydro-2H-pyran-2-yl)-3-(2-(4-chlorophenyl)acetamido)propane-1,2-diyl diacetate (ST-36_Int-5)(2.0 g, 2.82 mmol) and tert-butyl (21-hydroxy-15-oxo-3,6,9,12-tetraoxa-16-azahenicosyl)carbamate (4) (1.91 g, 1.41 mmol) in anhydrous dichloromethane (12 mL) and anhydrous acetonitrile (24 mL) were added Silver (+1) trifluoro methane sulfonate (Ag(OTf) (1.80 g, 7.07 mmol) and activated 4 Å powdered molecular sieves (5 g) and the reaction mixture was stirred at room temperature for 1 h under nitrogen atmosphere. Then the reaction mixture was cooled to −78° C. and iodine monobromide (0.87 g, 4.24 mmol, 1.5 eq.) solution in dichloromethane (4 ml) was added dropwise. The reaction mixture was stirred at the same temperature for 2 h. After completion, the reaction mixture was quenched with triethylamine (5 mL), filtered, filtrate was washed by 10% Sodium thiosulfate solution, dried over sodium sulfate, filtered and concentrated. The crude residue obtained was purified by C-18 reverse phase column chromatography, using acetonitrile and water (+0.05%, TFA) as eluents to afford the desired product (1R,2R)-1-((2R,3R,4S,6R)-3-acetamido-4-acetoxy-6-((2,2-dimethyl-4,20-dioxo-3,8,11,14,17-pentaoxa-5,21-diazahexacosan-26-yl) oxy)-6-(methoxycarbonyl) tetrahydro-2H-pyran-2-yl)-3-(2-(4-chlorophenyl) acetamido) propane-1,2-diyl diacetate (6) as yellow solid. Yield: 1.2 g, 41.23%, LCMS (ESI±) m/z 1035.4 [M+H]⁺.

### Synthesis of (2R,4S,5R,6R)-5-acetamido-6-((1R,2R)-3-(2-(4-chlorophenyl) acetamido)-1,2-dihydroxypropyl)-2-((2,2-dimethyl-4,20-dioxo-3,8,11,14,17-pentaoxa-5,21-diazahexacosan-26-yl) oxy)-4-hydroxytetrahydro-2H-pyran-2-carboxylic acid (7)

To a stirred solution of (1R,2R)-1-((2R,3R,4S,6R)-3-acetamido-4-acetoxy-6-((2,2-dimethyl-4,20-dioxo-3,8,11,14,17-pentaoxa-5,21-diazahexacosan-26-yl) oxy)-6-(methoxycarbonyl) tetrahydro-2H-pyran-2-yl)-3-(2-(4-chlorophenyl) acetamido) propane-1,2-diyl diacetate (6) (1 g, 0.96 mmol) in tetrahydrofuran (5 mL) was added lithium hydroxide monohydrate (243 mg, 5.80 mmol) solution in water. The reaction mixture was stirred at room temperature for 16 h. After completion the reaction mixture was treated with acidic resin (Dowex 50, H⁺) to maintain pH˜6 and the suspension was filtered, the filtrate was concentrated, to afford the desired product (2R,4S,5R,6R)-5-acetamido-6-((1R,2R)-3-(2-(4-chlorophenyl) acetamido)-1,2-dihydroxypropyl)-2-((2,2-dimethyl-4,20-dioxo-3,8,11,14,17-pentaoxa-5,21-diazahexacosan-26-yl) oxy)-4-hydroxytetrahydro-2H-pyran-2-carboxylic acid (7). Yield: 850 mg, 98.37%; LCMS (ESI±) m/z 895.7 [M+H]⁺.

### Synthesis of (2R,4S,5R,6R)-5-acetamido-2-((1-amino-15-oxo-3,6,9,12-tetraoxa-16-azahenicosan-21-yl) oxy)-6-((1R,2R)-3-(2-(4-chlorophenyl) acetamido)-1,2-dihydroxypropyl)-4-hydroxytetrahydro-2H-pyran-2-carboxylic acid (8)

To a stirred solution of (2R,4S,5R,6R)-5-acetamido-6-((1R,2R)-3-(2-(4-chlorophenyl) acetamido)-1,2-dihydroxypropyl)-2-((2,2-dimethyl-4,20-dioxo-3,8,11,14,17-pentaoxa-5,21-diazahexacosan-26-yl) oxy)-4-hydroxytetrahydro-2H-pyran-2-carboxylic acid (7) (0.85 g, 9.51 mmol) in anhydrous dichloromethane (5 mL), was added trifluoroacetic acid (0.33 mL) at 0° C. The reaction mixture was stirred at room temperature for 2 h. After completion, the reaction mixture was concentrated to get crude material which was dissolved in methanol and neutralized with tetra alkyl ammonium carbonate, the polymer-bound resin. Reaction mixture was filtered and filtrate was concentrated to afford the desired product (2R,4S,5R,6R)-5-acetamido-2-((1-amino-15-oxo-3,6,9,12-tetraoxa-16-azahenicosan-21-yl) oxy)-6-((1R,2R)-3-(2-(4-chlorophenyl) acetamido)-1,2-dihydroxypropyl)-4-hydroxytetrahydro-2H-pyran-2-carboxylic acid (8) as yellow solid. Yield: 650 mg, 86.20%; LCMS (ESI±) m/z 796.6 [M+H]⁺.

### Synthesis of (2R,4S,5R,6R)-5-acetamido-6-((1R,2R)-3-(2-(4-chlorophenyl) acetamido)-1,2-dihydroxypropyl)-2-((2,18-dioxo-6,9,12,15-tetraoxa-3,19-diazatetracosan-24-yl) oxy)-4-hydroxytetrahydro-2H-pyran-2-carboxylic acid (249)

To a stirred solution of (2R,4S,5R,6R)-5-acetamido-2-((1-amino-15-oxo-3,6,9,12-tetraoxa-16-azahenicosan-21-yl)oxy)-6-((1R,2R)-3-(2-(4-chlorophenyl)acetamido)-1,2-dihydroxypropyl)-4-hydroxytetrahydro-2H-pyran-2-carboxylic acid (8) (100 mg, 0.12 mmol) in N,N-dimethylformamide (1 mL) was added 2,5-dioxopyrrolidin-1-yl acetate (9) (19.8 mg, 0.12 mmol) at 0° C. The resulting reaction mixture was stirred at the same temperature for 45 min. After completion, the reaction mixture was purified by prep-HPLC using acetonitrile and water (+0.05% TFA) to afford the desired product (2R,4S,5R,6R)-5-acetamido-6-((1R,2R)-3-(2-(4-chlorophenyl) acetamido)-1,2-dihydroxypropyl)-2-((2,18-dioxo-6,9,12,15-tetraoxa-3,19-diazatetracosan-24-yl) oxy)-4-hydroxytetrahydro-2H-pyran-2-carboxylic acid (249) as white solid. Yield: 16 mg, 15.23%; LCMS (ESI±) m/z 837.8 [M+H]⁺. ¹H NMR (400 MHz, MeOD): δ 7.32 (s, 4H), 3.90 (td, J=8.3, 3.1 Hz, 1H), 3.82-3.71 (m, 5H), 3.71-3.68 (m, 6H), 3.67-3.60 (m, 12H), 3.58-3.54 (m, 4H), 3.37-3.35 (m, 4H), 3.27 (dd, J=13.7, 7.8 Hz, 1H), 3.19 (t, J=6.9 Hz, 2H), 2.77 (dd, J=12.7, 5.0 Hz, 1H), 2.45 (t, J=6.1 Hz, 2H), 1.99 (s, 3H), 1.96 (s, 3H), 1.72 (t, J=11.8 Hz, 1H), 1.54 (dq, J=27.0, 6.8 Hz, 4H), 1.40 (p, J=6.6 Hz, 2H).

### (2R,4S,5R,6R)-5-acetamido-6-((1R,2R)-3-(2-(4-chlorophenyl) acetamido)-1,2-dihydroxypropyl)-2-((1-(2,5-dioxo-2,5-dihydro-1H-pyrrol-1-yl)-3,19-dioxo-7,10,13,16-tetraoxa-4,20-diazapentacosan-25-yl) oxy)-4-hydroxytetrahydro-2H-pyran-2-carboxylic acid (Cpd. 249M)

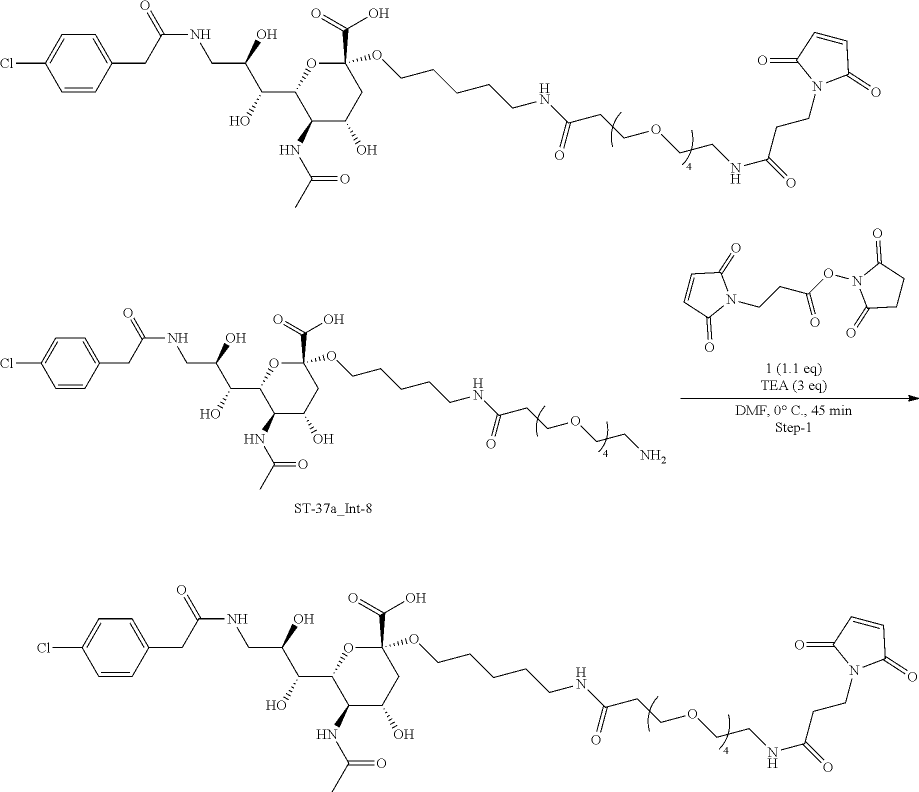

### Synthesis of (2R,4S,5R,6R)-5-acetamido-6-((1R,2R)-3-(2-(4-chlorophenyl) acetamido)-1,2-dihydroxypropyl)-2-((1-(2,5-dioxo-2,5-dihydro-1H-pyrrol-1-yl)-3,19-dioxo-7,10,13,16-tetraoxa-4,20-diazapentacosan-25-yl) oxy)-4-hydroxytetrahydro-2H-pyran-2-carboxylic acid (249M)

To a stirred solution (2R,4S,5R,6R)-5-acetamido-2-((1-amino-15-oxo-3,6,9,12-tetraoxa-16-azahenicosan-21-yl)oxy)-6-((1R,2R)-3-(2-(4-chlorophenyl)acetamido)-1,2-dihydroxypropyl)-4-hydroxytetrahydro-2H-pyran-2-carboxylic acid (249_Int-8) (200 mg, 0.25 mmol) in N,N-dimethyl formamide (2 mL) were added 2,5-dioxopyrrolidin-1-yl 3-(2,5-dioxo-2,5-dihydro-1H-pyrrol-1-yl)propanoate (1) (67 mg, 0.12 mmol) and triethylamine (0.1 ml, 0.75 mmol) at 0° C. The resulting reaction mixture was stirred at 0° C. for 45 min. After completion, the reaction mixture was purified by prep-HPLC using acetonitrile and water (+0.05%, TFA) as eluents to afford the desired product (2R,4S,5R,6R)-5-acetamido-6-((1R,2R)-3-(2-(4-chlorophenyl) acetamido)-1,2-dihydroxypropyl)-2-((1-(2,5-dioxo-2,5-dihydro-1H-pyrrol-1-yl)-3,19-dioxo-7,10,13,16-tetraoxa-4,20-diazapentacosan-25-yl) oxy)-4-hydroxytetrahydro-2H-pyran-2-carboxylic acid (249M) as white solid. Yield: 70 mg, 29.41%; LCMS (ESI±) m/z 945.8 [M+H]⁺. ¹H NMR (400 MHz, MeOD) δ 7.31 (s, 4H), 6.83 (s, 2H), 3.94-3.85 (m, 1H), 3.85-3.71 (m, 6H), 3.71-3.58 (m, 15H), 3.58-3.48 (m, 4H), 3.38-3.15 (m, 6H), 2.78-2.67 (m, 1H), 2.47 (dt, J=13.3, 6.5 Hz, 4H), 1.99 (s, 3H), 1.73 (t, J=12.1 Hz, 1H), 1.63-1.34 (m, 6H).

### (2R,4S,5R,6R)-5-acetamido-6-((1R,2R)-3-(2-(4-chlorophenyl) acetamido)-1,2-dihydroxypropyl)-4-hydroxy-2-((5,21,37-trioxo-1-((3aR,4R,6aS)-2-oxohexahydro-1H-thieno[3,4-d] imidazol-4-yl)-9,12,15,18,25,28,31,34-octaoxa-6,22,38-triazatritetracontan-43-yl) oxy) tetrahydro-2H-pyran-2-carboxylic acid (Cpd. 249B)

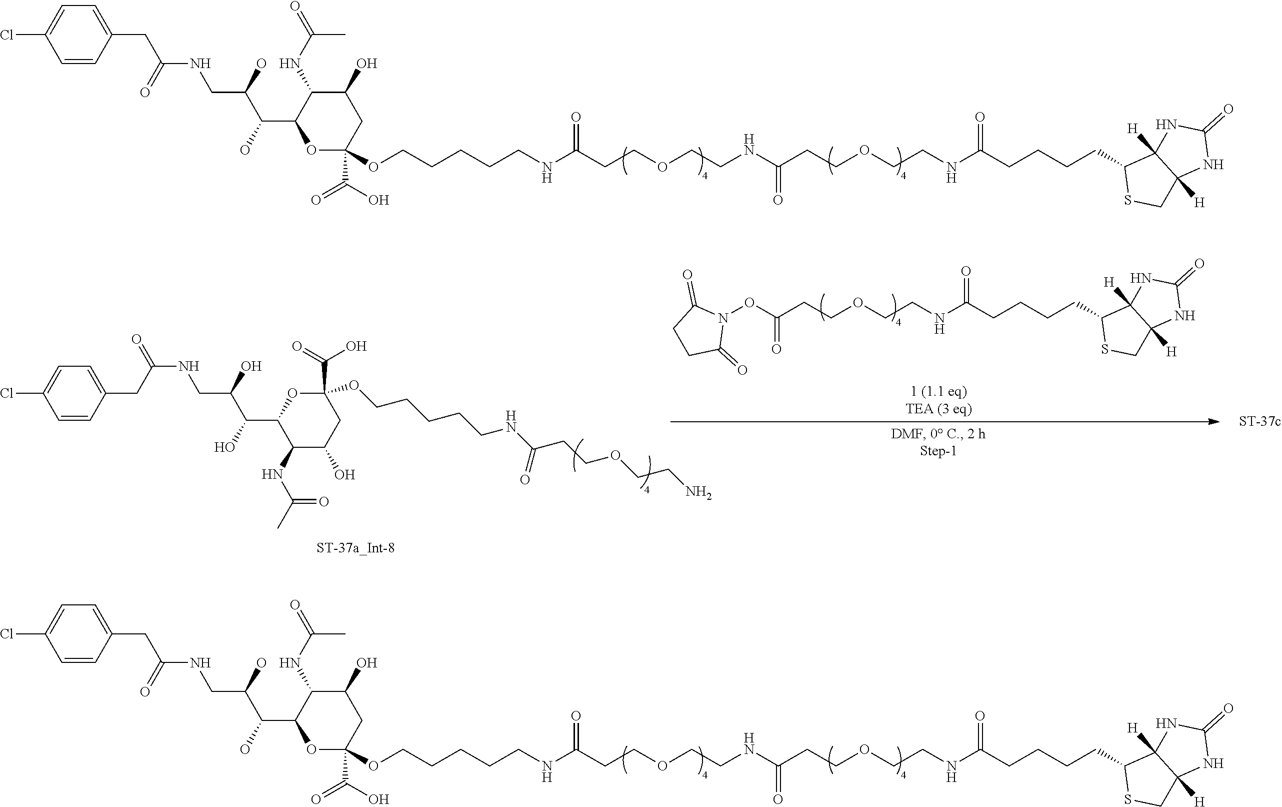

### Synthesis of (2R,4S,5R,6R)-5-acetamido-6-((1R,2R)-3-(2-(4-chlorophenyl) acetamido)-1,2-dihydroxypropyl)-4-hydroxy-2-((5,21,37-trioxo-1-((3aR,4R,6aS)-2-oxohexahydro-1H-thieno[3,4-d] imidazol-4-yl)-9,12,15,18,25,28,31,34-octaoxa-6,22,38-triazatritetracontan-43-yl) oxy) tetrahydro-2H-pyran-2-carboxylic acid (249B)

To a stirred solution (2R,4S,5R,6R)-5-acetamido-2-((1-amino-15-oxo-3,6,9,12-tetraoxa-16-azahenicosan-21-yl)oxy)-6-((1R,2R)-3-(2-(4-chlorophenyl)acetamido)-1,2-dihydroxypropyl)-4-hydroxytetrahydro-2H-pyran-2-carboxylic acid (249_Int-8)(50 mg, 0.06 mmol) in N,N-Dimethyl formamide (0.5 mL) were added 2,5-dioxopyrrolidin-1-yl 17-oxo-21-((3aR,4R,6aS)-2-oxohexahydro-1H-thieno[3,4-d]imidazol-4-yl)-4,7,10,13-tetraoxa-16-azahenicosanoate (1) (37 mg, 0.06 mmol) and triethylamine (0.02 ml, 0.01 mmol) at 0° C. and the reaction mixture was stirred for 2 h at the same temperature. After completion, the reaction mixture was purified by prep-HPLC purification, using acetonitrile and water (+0.05%, TFA) as eluents to afford the desired product (2R,4S,5R,6R)-5-acetamido-6-((1R,2R)-3-(2-(4-chlorophenyl) acetamido)-1,2-dihydroxypropyl)-4-hydroxy-2-((5,21,37-trioxo-1-((3aR,4R,6aS)-2-oxohexahydro-1H-thieno[3,4-d] imidazol-4-yl)-9,12,15,18,25,28,31,34-octaoxa-6,22,38-triazatritetracontan-43-yl) oxy) tetrahydro-2H-pyran-2-carboxylic acid (249B) as white solid. Yield: 19 mg, 23.80%; LCMS (ESI±) m/z 1265.1 [M−H]⁻. ¹H NMR (400 MHz, MeOD) δ 8.03 (s, 1H), 7.32 (s, 4H), 3.74 (td, J=6.1, 3.1 Hz, 7H), 3.71-3.64 (m, 22H), 3.64-3.51 (m, 13H), 3.39 (q, J=5.3 Hz, 7H), 3.30-3.15 (m, 6H), 2.75 (dd, J=21.1, 12.7 Hz, 3H), 2.47 (dt, J=7.9, 6.1 Hz, 4H), 2.24 (t, J=7.4 Hz, 2H), 2.00 (s, 3H), 1.81-1.53 (m, 9H), 1.45 (ddd, J=29.5, 13.8, 7.2 Hz, 6H).

### (2R,4S,5R,6R)-5-acetamido-6-((1R,2R)-3-(2-(4-chlorophenyl) acetamido)-1,2-dihydroxypropyl)-4-hydroxy-2-((6-(4-(15-oxo-2,5,8,11-tetraoxa-14-azahexadecyl)-1H-1,2,3-triazol-1-yl) hexyl)oxy) tetrahydro-2H-pyran-2-carboxylic acid (Cpd. 250)

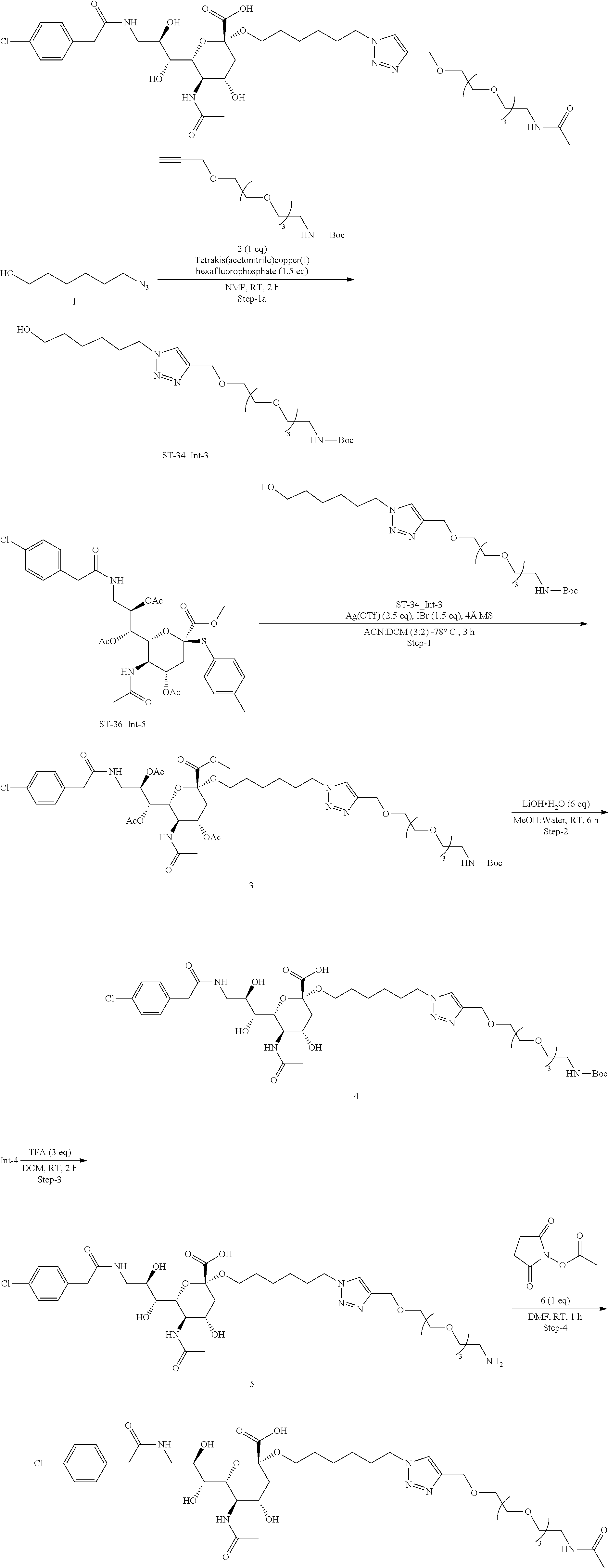

### Synthesis of tert-butyl (1-(1-(6-hydroxyhexyl)-1H-1,2,3-triazol-4-yl)-2,5,8,11-tetraoxatridecan-13-yl) carbamate (ST-34_Int-3)

To a stirred solution of 6-azidohexan-1-ol (1) (2 g, 13.9 mmol) and tert-butyl (3,6,9,12-tetraoxapentadec-14-yn-1-yl) carbamate (2) (4.6 g, 13.9 mmol) in N-Methyl-2-Pyrrilidone (20 mL) was added tetrakis (acetonitrile) copper (I) hexafluoro phosphate (7.8 g, 20.9 mmol) and the reaction mixture was stirred at room temperature for 2 h. After completion, the reaction mixture was concentrated to get the crude material, which was purified by C-18 reverse phase column chromatography using acetonitrile and water (+0.05%, TFA) as eluents to afford tert-butyl (1-(1-(6-hydroxyhexyl)-1H-1,2,3-triazol-4-yl)-2,5,8,11-tetraoxatridecan-13-yl) carbamate (ST-34_Int-3) as colorless semi solid. Yield: 2.5 g, 37.71%.

¹H NMR (400 MHz, DMSO): δ 8.09 (S, 1H), 6.76 (s, 1H), 4.50 (s, 2H), 4.33 (t, J=6.8, 2H), 3.53-3.29 (m, 14H), 3.05 (d, J=506, 2H), 1.80 (t, J=7.2, 2H), 1.51 (d, J=7.6, 1H), 1.39-1.30 (m, 17H).

### Synthesis of (1R,2R)-1-((2R,3R,4S,6R)-3-acetamido-4-acetoxy-6-((6-(4-(17,17-dimethyl-15-oxo-2,5,8,11,16-pentaoxa-14-azaoctadecyl)-1H-1,2,3-triazol-1-yl) hexyl)oxy)-6-(methoxycarbonyl) tetrahydro-2H-pyran-2-yl)-3-(2-(4-chlorophenyl) acetamido)propane-1,2-diyl diacetate (3)

To a stirred solution of (1R,2R)-1-((2R,3R,4S,6R)-3-acetamido-4-acetoxy-6-(methoxycarbonyl)-6-(p-tolylthio)tetrahydro-2H-pyran-2-yl)-3-(2-(4-chlorophenyl)acetamido)propane-1,2-diyl diacetate (ST-36_Int-5) (2.0 g, 2.82 mmol) and tert-butyl (1-(1-(6-hydroxyhexyl)-1H-1,2,3-triazol-4-yl)-2,5,8,11-tetraoxatridecan-13-yl)carbamate (ST-34_Int-3) (2.14 g, 4.24 mmol) in anhydrous dichloromethane (12 mL) and anhydrous acetonitrile (24 mL) were added Silver (+1) trifluoro methane sulfonate Ag(OTf) (1.80 g, 7.07 mmol) and activated 4 Å powdered molecular sieves (5 g) and the reaction mixture was stirred at room temperature for 1 h under nitrogen atmosphere. Then the solution was cooled to −78° C. and iodine monobromide (0.87 g, 4.24 mmol) solution in dichloromethane (4 ml) was added dropwise to the reaction mixture and was stirred at the same temperature for 2 h. After completion, the reaction mixture was quenched by triethyl amine (5 mL) and warmed to room temperature. The reaction mixture was filtered, filtrate was washed by 10% Sodium thiosulfate solution, dried over sodium sulfate, filtered and concentrated to get the crude material, which was purified by C-18 reverse phase column chromatography using acetonitrile and water (+0.05%, TFA) as eluents to afford the desired product (1R,2R)-1-((2R,3R,4S,6R)-3-acetamido-4-acetoxy-6-((6-(4-(17,17-dimethyl-15-oxo-2,5,8,11,16-pentaoxa-14-azaoctadecyl)-1H-1,2,3-triazol-1-yl) hexyl)oxy)-6-(methoxycarbonyl) tetrahydro-2H-pyran-2-yl)-3-(2-(4-chlorophenyl) acetamido) propane-1,2-diyl diacetate (3) as yellow solid. Yield: 1.4 g, 46.82%; (LCMS (ESI±) m/z 1057.59 [M+H]⁺.

### Synthesis of (2R,4S,5R,6R)-5-acetamido-6-((1R,2R)-3-(2-(4-chlorophenyl) acetamido)-1,2-dihydroxypropyl)-2-((6-(4-(17,17-dimethyl-15-oxo-2,5,8,11,16-pentaoxa-14-azaoctadecyl)-1H-1,2,3-triazol-1-yl) hexyl)oxy)-4-hydroxytetrahydro-2H-pyran-2-carboxylic acid (7)

To a stirred solution of (1R,2R)-1-((2R,3R,4S,6R)-3-acetamido-4-acetoxy-6-((6-(4-(17,17-dimethyl-15-oxo-2,5,8,11,16-pentaoxa-14-azaoctadecyl)-1H-1,2,3-triazol-1-yl) hexyl) oxy)-6-(methoxycarbonyl) tetrahydro-2H-pyran-2-yl)-3-(2-(4-chlorophenyl) acetamido) propane-1,2-diyl diacetate (3) (1.1 g, 1.04 mmol) in tetrahydrofuran (10 mL) was added lithium hydroxide monohydrate (87 mg, 6.24 mmol) solution in water (3 mL) at 0° C. and the reaction mixture was stirred at room temperature for 6 h. After completion the reaction mixture was treated with acidic resin (Dowex 50, H⁺) to maintain pH˜6. The suspension was filtered, filtrate was concentrated to get the crude residue, which was purified by C-18 reverse phase column chromatography using acetonitrile and water (+0.05%, TFA) as eluents to afford the desired product (2R,4S,5R,6R)-5-acetamido-6-((1R,2R)-3-(2-(4-chlorophenyl) acetamido)-1,2-dihydroxypropyl)-2-((6-(4-(17,17-dimethyl-15-oxo-2,5,8,11,16-pentaoxa-14-azaoctadecyl)-1H-1,2,3-triazol-1-yl) hexyl)oxy)-4-hydroxytetrahydro-2H-pyran-2-carboxylic acid (4) as off white solid. Yield: 510 mg, 54.40%; LCMS (ESI±) m/z 918.8 [M+H]⁺.

### Synthesis of (2R,4S,5R,6R)-5-acetamido-2-((6-(4-(13-amino-2,5,8,11-tetraoxatridecyl)-1H-1,2,3-triazol-1-yl) hexyl)oxy)-6-((1R,2R)-3-(2-(4-chlorophenyl) acetamido)-1,2-dihydroxypropyl)-4-hydroxytetrahydro-2H-pyran-2-carboxylic acid (5)

To a stirred solution of (2R,4S,5R,6R)-5-acetamido-6-((1R,2R)-3-(2-(4-chlorophenyl) acetamido)-1,2-dihydroxypropyl)-2-((6-(4-(17,17-dimethyl-15-oxo-2,5,8,11,16-pentaoxa-14-azaoctadecyl)-1H-1,2,3-triazol-1-yl) hexyl)oxy)-4-hydroxytetrahydro-2H-pyran-2-carboxylic acid (4) (0.8 g, 0.8 mmol) in anhydrous dichloromethane (8 mL) was added trifluoro acetic acid (0.8 mL) at 0° C. Then the reaction mixture was stirred at room temperature for 2 h. After completion, the reaction mixture was concentrated, the crude was suspended in methanol and neutralized with Tetra alkyl ammonium carbonate, the polymer-bound resin. The reaction mixture was filtered and filtrate was concentrated to afford the desired product (2R,4S,5R,6R)-5-acetamido-2-((1-amino-15-oxo-3,6,9,12-tetraoxa-16-azahenicosan-21-yl) oxy)-6-((1R,2R)-3-(2-(4-chlorophenyl) acetamido)-1,2-dihydroxypropyl)-4-hydroxytetrahydro-2H-pyran-2-carboxylic acid (5) as yellow solid. Yield: 700 mg, 71%; LCMS (ESI±) m/z 818.7 [M+H]⁺.

### Synthesis of (2R,4S,5R,6R)-5-acetamido-6-((1R,2R)-3-(2-(4-chlorophenyl) acetamido)-1,2-dihydroxypropyl)-4-hydroxy-2-((6-(4-(15-oxo-2,5,8,11-tetraoxa-14-azahexadecyl)-1H-1,2,3-triazol-1-yl) hexyl)oxy) tetrahydro-2H-pyran-2-carboxylic acid (250)

To a stirred solution of (2R,4S,5R,6R)-5-acetamido-2-((1-amino-15-oxo-3,6,9,12-tetraoxa-16-azahenicosan-21-yl)oxy)-6-((1R,2R)-3-(2-(4-chlorophenyl)acetamido)-1,2-dihydroxypropyl)-4-hydroxytetrahydro-2H-pyran-2-carboxylic acid (5) (80 mg, 0.97 mmol) in N,N-Dimethyl formamide (1 mL) was added 2,5-dioxopyrrolidin-1-yl acetate (6) (15.3 mg, 0.97 mmol) at 0° C. The resulting reaction mixture was stirred at room temperature for 1 h. After completion, the reaction mixture was purified by prep-HPLC using acetonitrile and water (+0.05%, TFA) as eluents to afford the desired product (2R,4S,5R,6R)-5-acetamido-6-((1R,2R)-3-(2-(4-chlorophenyl) acetamido)-1,2-dihydroxypropyl)-4-hydroxy-2-((6-(4-(15-oxo-2,5,8,11-tetraoxa-14-azahexadecyl)-1H-1,2,3-triazol-1-yl) hexyl)oxy) tetrahydro-2H-pyran-2-carboxylic acid (250) as white solid. Yield: 12 mg, 15.23%; LCMS (ESI±) m/z 859.55 [M+H]⁺. ¹H NMR (400 MHz, MeOD) δ 8.00 (s, 1H), 7.31 (d, J=1.9 Hz, 4H), 4.66 (s, 2H), 4.42 (t, J=7.2 Hz, 2H), 3.89 (t, J=9.3 Hz, 2H), 3.77-3.58 (m, 16H), 3.58-3.53 (m, 5H), 3.37 (m, 3H), 3.32-3.22 (m, 1H), 2.71 (dd, J=12.7, 4.2 Hz, 1H), 2.01 (s, 3H), 1.96 (s, 3H), 1.94 (t, J=7.6 Hz, 2H), 1.73 (t, J=12.0 Hz, 1H), 1.55 (d, J=6.9 Hz, 2H), 1.42 (s, 2H), 1.34 (q, J=6.1 Hz, 2H).

### (2R,4S,5R,6R)-5-acetamido-6-((1R,2R)-3-(2-(4-chlorophenyl) acetamido)-1,2-dihydroxypropyl)-2-((6-(4-(17-(2,5-dioxo-2,5-dihydro-1H-pyrrol-1-yl)-15-oxo-2,5,8,11-tetraoxa-14-azaheptadecyl)-1H-1,2,3-triazol-1-yl) hexyl)oxy)-4-hydroxytetrahydro-2H-pyran-2-carboxylic acid (Cpd. 250M)

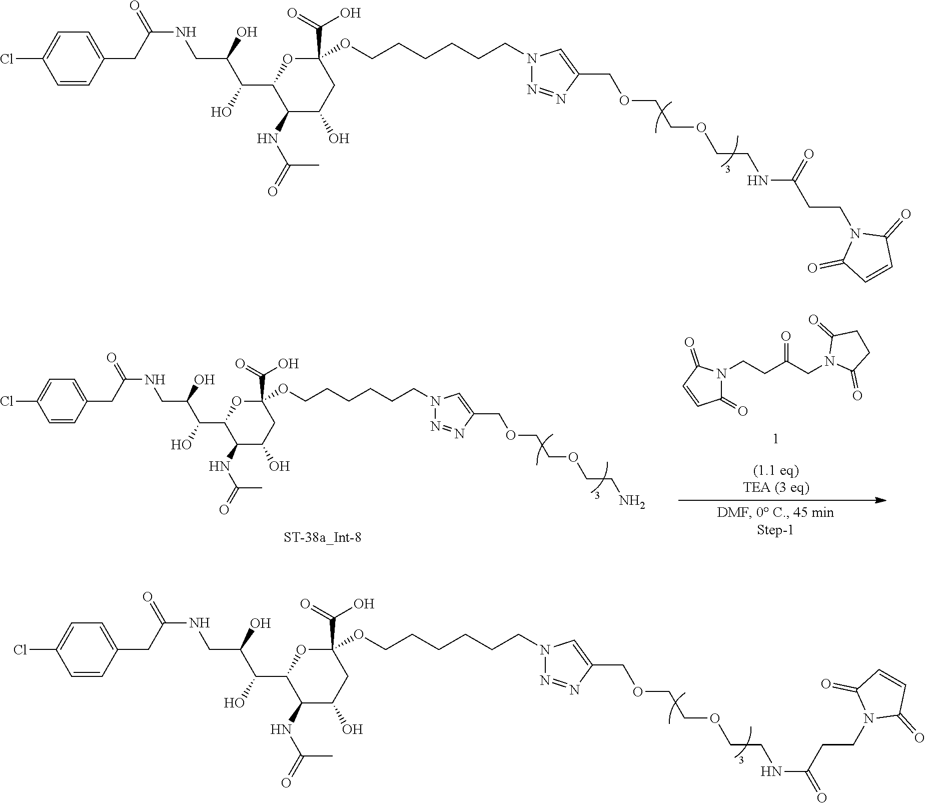

### Synthesis of (2R,4S,5R,6R)-5-acetamido-6-((1R,2R)-3-(2-(4-chlorophenyl) acetamido)-1,2-dihydroxypropyl)-2-((6-(4-(17-(2,5-dioxo-2,5-dihydro-1H-pyrrol-1-yl)-15-oxo-2,5,8,11-tetraoxa-14-azaheptadecyl)-1H-1,2,3-triazol-1-yl) hexyl)oxy)-4-hydroxytetrahydro-2H-pyran-2-carboxylic acid (250M)

To a stirred solution of (2R,4S,5R,6R)-5-acetamido-2-((6-(4-(13-amino-2,5,8,11-tetraoxatridecyl)-1H-1,2,3-triazol-1-yl)hexyl)oxy)-6-((1R,2R)-3-(2-(4-chlorophenyl)acetamido)-1,2-dihydroxypropyl)-4-hydroxytetrahydro-2H-pyran-2-carboxylic acid (250_Int-5) (200 mg, 0.24 mmol) and 2,5-dioxopyrrolidin-1-yl acetate (1) (71.6 mg, 0.26 mmol) in N,N-dimethylformamide (2 mL) was added triethyl amine (0.11 ml, 0.73 mmol) at 0° C. and the resulting reaction mixture was stirred at the same temperature for 45 min. After completion, the reaction mixture was purified by prep-HPLC using acetonitrile and water (+0.05% TFA) as eluents to afford the desired product (2R,4S,5R,6R)-5-acetamido-6-((1R,2R)-3-(2-(4-chlorophenyl) acetamido)-1,2-dihydroxypropyl)-2-((6-(4-(17-(2,5-dioxo-2,5-dihydro-1H-pyrrol-1-yl)-15-oxo-2,5,8,11-tetraoxa-14-azaheptadecyl)-1H-1,2,3-triazol-1-yl) hexyl)oxy)-4-hydroxytetrahydro-2H-pyran-2-carboxylic acid (250M) as white solid. Yield: 34 mg, 14.34%; LCMS (ESI±) m/z 970.4 [M+H]⁺. ¹H NMR (401 MHz, MeOD) δ 7.99 (d, J=3.2 Hz, 1H), 7.31 (s, 4H), 6.83 (s, 2H), 4.66 (s, 2H), 4.42 (t, J=7.2 Hz, 2H), 3.93-3.47 (m, 14), 3.45-3.34 (m, 5H), 3.33-3.21 (m, 7H), 2.71 (dd, J=12.7, 4.2 Hz, 1H), 2.48 (t, J=6.9 Hz, 4H), 2.00 (s, 3H), 1.91 (p, J=6.8 Hz, 3H), 1.78-1.49 (m, 3H), 1.40 (s, 2H), 1.38-1.29 (m, 2H).

### (2R,4S,5R,6R)-5-acetamido-6-((1R,2R)-3-(2-(4-chlorophenyl) acetamido)-1,2-dihydroxypropyl)-2-((6-(4-(15,31-dioxo-35-((3aR,4R,6aS)-2-oxohexahydro-1H-thieno[3,4-d] imidazol-4-yl)-2,5,8,11,18,21,24,27-octaoxa-14,30-diazapentatriacontyl)-1H-1,2,3-triazol-1-yl) hexyl)oxy)-4-hydroxytetrahydro-2H-pyran-2-carboxylic acid (Cpd. No. 250B)

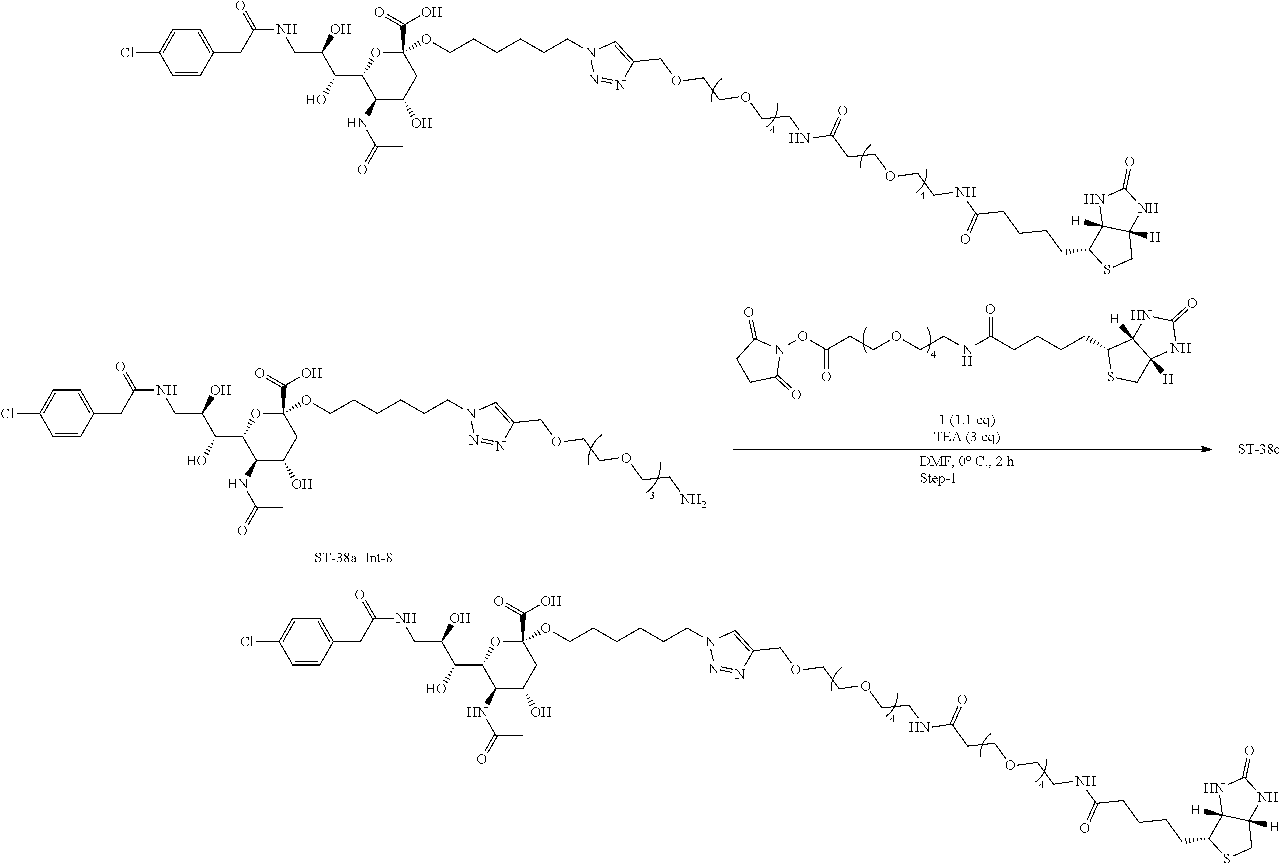

### Synthesis of (2R,4S,5R,6R)-5-acetamido-6-((1R,2R)-3-(2-(4-chlorophenyl) acetamido)-1,2-dihydroxypropyl)-2-((6-(4-(15,31-dioxo-35-((3aR,4R,6aS)-2-oxohexahydro-1H-thieno[3,4-d]imidazol-4-yl)-2,5,8,11,18,21,24,27-octaoxa-14,30-diazapentatriacontyl)-1H-1,2,3-triazol-1-yl) hexyl)oxy)-4-hydroxytetrahydro-2H-pyran-2-carboxylic acid (250B)

To a stirred solution of (2R,4S,5R,6R)-5-acetamido-2-((6-(4-(13-amino-2,5,8,11-tetraoxatridecyl)-1H-1,2,3-triazol-1-yl)hexyl)oxy)-6-((1R,2R)-3-(2-(4-chlorophenyl)acetamido)-1,2-dihydroxypropyl)-4-hydroxytetrahydro-2H-pyran-2-carboxylic acid (250_Int-5) (50 mg, 0.06 mmol) and 2,5-dioxopyrrolidin-1-yl 17-oxo-21-((3aR,4R,6aS)-2-oxohexahydro-1H-thieno[3,4-d]imidazol-4-yl)-4,7,10,13-tetraoxa-16-azahenicosanoate (1) (39.6 mg, 0.067 mmol) in N,N-Dimethyl formamide (0.5 mL) was added triethyl amine (0.05 ml, 0.18 mmol) at 0° C. and the resulting reaction mixture was stirred at room temperature for 2 h. After completion, the reaction mixture was purified by prep-HPLC using acetonitrile and water (+0.05%, TFA) as eluents to afford the desired product (2R,4S,5R,6R)-5-acetamido-6-((1R,2R)-3-(2-(4-chlorophenyl) acetamido)-1,2-dihydroxypropyl)-2-((6-(4-(17-(2,5-dioxo-2,5-dihydro-1H-pyrrol-1-yl)-15-oxo-2,5,8,11-tetraoxa-14-azaheptadecyl)-1H-1,2,3-triazol-1-yl) hexyl)oxy)-4-hydroxytetrahydro-2H-pyran-2-carboxylic acid (250M) as white solid. Yield: 14 mg, 17.94%; LCMS (ESI±) m/z 1291.2 [M+H]+. 1H NMR (401 MHz, MeOD) δ 8.00 (s, 1H), 7.31 (d, J=1.9 Hz, 4H), 4.66 (s, 2H), 4.51 (dd, J=7.9, 5.0 Hz, 1H), 4.41 (dd, J=8.2, 6.1 Hz, 2H), 4.32 (dd, J=8.0, 4.5 Hz, 1H), 3.86 (t, J=9.2 Hz, 2H), 3.79-3.68 (m, 5H), 3.67 (d, J=11.5 Hz, 16H), 3.63-3.52 (m, 7H), 3.46-3.30 (m, 5H), 3.24 (ddd, J=15.2, 12.0, 6.8 Hz, 3H), 2.94 (dd, J=12.7, 5.0 Hz, 1H), 2.76-2.66 (m, 2H), 2.47 (t, J=6.2 Hz, 2H), 2.24 (t, J=7.4 Hz, 2H), 2.00 (d, J=6.2 Hz, 3H), 1.91 (t, J=7.3 Hz, 2H), 1.80-1.51 (m, 6H), 1.45 (dq, J=13.3, 6.6 Hz, 2H), 1.33 (d, J=9.0 Hz, 3H).

### (2S,4S,5R,6R)-6-((1R,2R)-3-(4-chlorobenzamido)-1,2-dihydroxypropyl)-4-hydroxy-5-(2-hydroxyacetamido)-2-((6-(prop-2-yn-1-yloxy) hexyl) thio) tetrahydro-2H-pyran-2-carboxylic acid (Cpd. 251)

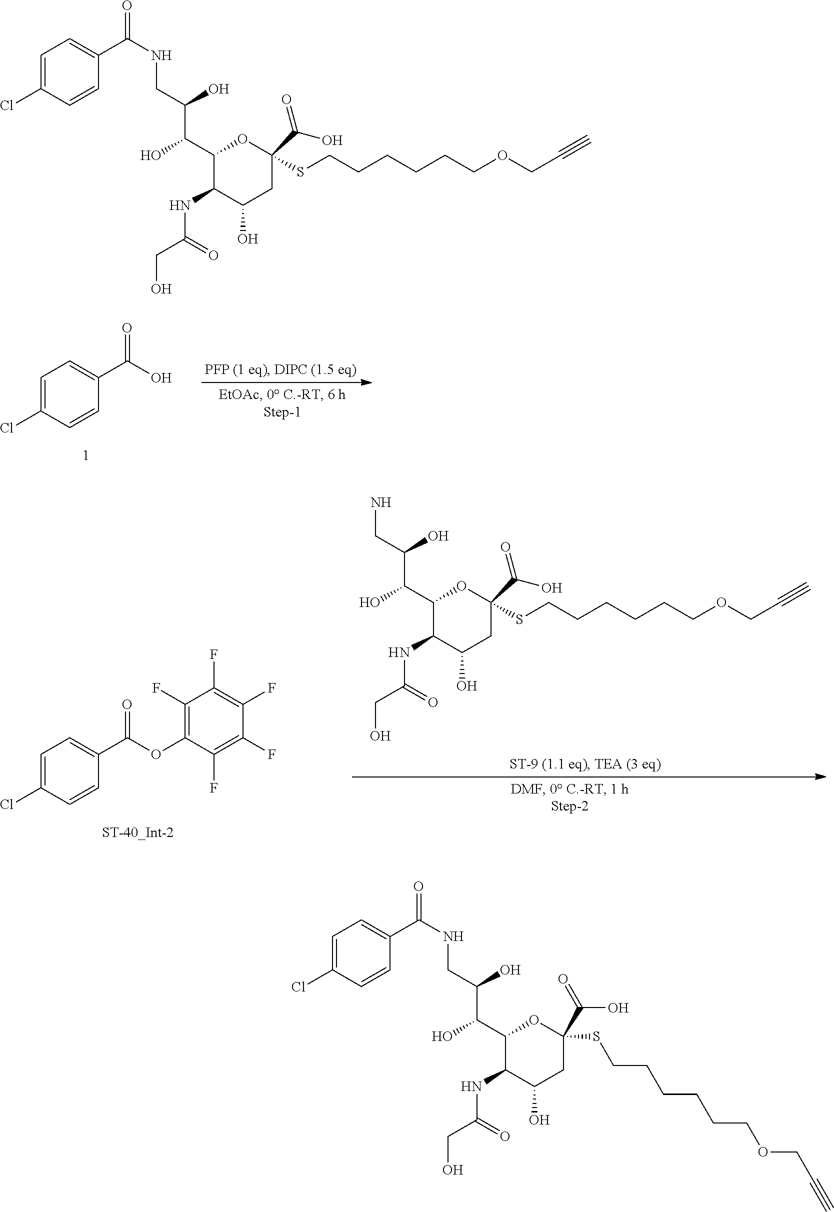 [SMILES: C#CCOCCCCCCS[C@]1(C(=O)O)C[C@H](O)[C@@H](NC(=O)CO)[C@H]([C@H](O)[C@H](O)CNC(=O)c2ccc(Cl)cc2)O1 ; O=C(O)c1ccc(Cl)cc1 ; O=C(Oc1c(F)c(F)c(F)c(F)c1F)c1ccc(Cl)cc1 ; C#CCOCCCCCCS[C@]1(C(=O)O)C[C@H](O)[C@@H](NC(=O)CO)[C@H]([C@H](O)[C@H](O)CN)O1 ; C#CCOCCCCCCS[C@]1(C(=O)O)C[C@H](O)[C@@H](NC(=O)CO)[C@H]([C@H](O)[C@H](O)CNC(=O)c2ccc(Cl)cc2)O1]

### Synthesis of perfluorophenyl 4-chlorobenzoate (ST-40_Int-2)

To a stirred solution of 4-chlorobenzoic acid (1) (5 g, 31.94 mmol) and penta-fluoro phenol (5.88 g, 31.94 mmol) in dry ethyl acetate (50 mL) was added N, N′-di-isopropyl carbodiimide (6.0 g, 47.9 mmol) dropwise at 0° C. The reaction mixture was stirred at room temperature for 6 h. After completion, the reaction mixture was filtered, filtrate was concentrated to get the crude material, which was purified by Silica gel column chromatography using ethyl acetate and hexane as eluents to afford perfluorophenyl 4-chlorobenzoate (ST-40_Int-2) as a white solid. Yield: 3.5 g, 34%. ¹H NMR (400 MHz, DMSO) δ 8.24-8.16 (m, 2H), 7.82-7.72 (m, 2H).

### Synthesis of (2S,4S,5R,6R)-6-((1R,2R)-3-(4-chlorobenzamido)-1,2-dihydroxypropyl)-4-hydroxy-5-(2-hydroxyacetamido)-2-((6-(prop-2-yn-1-yloxy) hexyl) thio) tetrahydro-2H-pyran-2-carboxylic acid (Cpd. 251)

To a stirred solution of (ST-9) (100 mg, 0.21 mmol) in N,N-Dimethyl formamide (1 mL) were added perfluorophenyl 4-chlorobenzoate (ST-40_Int-2) (74 mg, 0.23 mmol) and triethylamine (63 mg, 0.63 mmol) at 0° C. The resulting reaction mixture was stirred at room temperature for 1 h. After completion, the reaction mixture was concentrated and purified by prep-HPLC using acetonitrile and water (+0.05%, TFA) as eluents to afford the desired product (2S,4S,5R,6R)-6-((1R,2R)-3-(4-chlorobenzamido)-1,2-dihydroxypropyl)-4-hydroxy-5-(2-hydroxyacetamido)-2-((6-(prop-2-yn-1-yloxy) hexyl) thio) tetrahydro-2H-pyran-2-carboxylic acid (251) as off white solid. Yield: 70 mg, 54%; LCMS (ESI±) m/z 617.0 [M+H]⁺.

¹H NMR (400 MHz, MeOD) δ 7.94 (d, J=6.9 Hz, 1H), 7.89-7.80 (m, 2H), 7.54-7.45 (m, 2H), 4.10 (d, J=2.4 Hz, 2H), 4.03 (s, 2H), 4.01-3.96 (m, 1H), 3.86 (tdd, J=13.7, 12.1, 6.7 Hz, 3H), 3.71-3.63 (m, 1H), 3.62-3.47 (m, 2H), 3.49-3.41 (m, 3H), 2.85-2.73 (m, 3H), 2.65 (ddd, J=12.6, 8.3, 6.7 Hz, 1H), 1.86-1.76 (m, 1H), 1.61 (dt, J=14.4, 7.4 Hz, 2H), 1.51 (p, J=6.9 Hz, 2H), 1.43-1.29 (m, 4H).

### (2R,4S,5R,6R)-6-((1R,2R)-3-(4-chlorobenzamido)-1,2-dihydroxypropyl)-2-((6-((3,4-dioxo-2-((2-(2-(prop-2-yn-1-yloxy) ethoxy)ethyl)amino) cyclobut-1-en-1-yl)amino)hexyl)oxy)-4-hydroxy-5-(2-hydroxyacetamido) tetrahydro-2H-pyran-2-carboxylic acid (Cpd. 254)

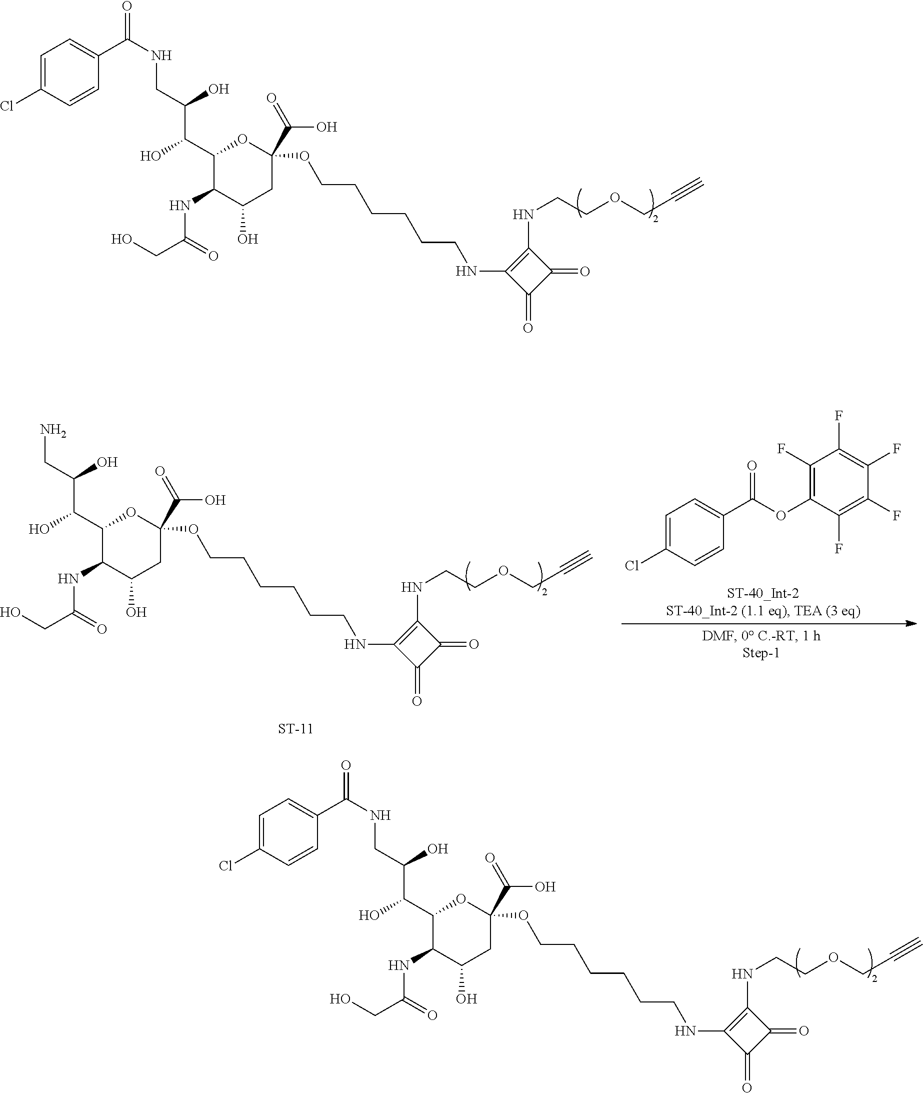

### Synthesis of (2R,4S,5R,6R)-6-((1R,2R)-3-(4-chlorobenzamido)-1,2-dihydroxypropyl)-2-((6-((3,4-dioxo-2-((2-(2-(prop-2-yn-1-yloxy) ethoxy)ethyl)amino) cyclobut-1-en-1-yl)amino)hexyl)oxy)-4-hydroxy-5-(2-hydroxyacetamido) tetrahydro-2H-pyran-2-carboxylic acid (Cpd. 254)

To a stirred solution of (2R,4S,5R,6R)-6-((1R,2R)-3-amino-1,2-dihydroxypropyl)-2-((6-((3,4-dioxo-2-((2-(2-(prop-2-yn-1-yloxy)ethoxy)ethyl)amino)cyclobut-1-en-1-yl)amino)hexyl)oxy)-4-hydroxy-5-(2-hydroxyacetamido)tetrahydro-2H-pyran-2-carboxylic acid (ST-11) (100 mg, 0.155 mmol) in N,N-Dimethyl formamide (1 mL) were added perfluorophenyl 4-chlorobenzoate (251_Int-2) (55 mg, 0.170 mmol) and triethylamine (47 mg, 0.47 mmol) at 0° C. The resulting reaction mixture was stirred at room temperature for 1 h. After completion, the reaction mixture was purified by prep-HPLC using acetonitrile and water (+0.05%, TFA) as eluents to afford the desired product (2R,4S,5R,6R)-6-((1R,2R)-3-(4-chlorobenzamido)-1,2-dihydroxypropyl)-2-((6-((3,4-dioxo-2-((2-(2-(prop-2-yn-1-yloxy) ethoxy)ethyl)amino) cyclobut-1-en-1-yl)amino)hexyl)oxy)-4-hydroxy-5-(2-hydroxyacetamido) tetrahydro-2H-pyran-2-carboxylic acid (254) as off white solid. Yield: 95 mg, 78%; LCMS (ESI±) m/z 783.5 [M+H]⁺.

¹H NMR (400 MHz, MeOD) δ 7.91 (d, J=6.9 Hz, 1H), 7.87-7.80 (m, 2H), 7.52-7.45 (m, 2H), 4.19 (d, J=2.5 Hz, 2H), 4.03 (s, 3H), 3.88-3.75 (m, 6H), 3.68 (q, J=1.3 Hz, 3H), 3.65 (q, J=4 Hz, 3H), 3.62-3.56 (m, 2H), 3.56-3.38 (m, 4H), 2.86 (t, J=2.4 Hz, 1H), 2.72 (dd, J=12.7, 3.7 Hz, 1H), 1.75 (t, J=12.1 Hz, 1H), 1.65-1.52 (m, 4H), 1.45-1.34 (m, 4H).

### (2S,4S,5R,6R)-6-((1R,2R)-1,2-dihydroxy-3-(3-phenoxybenzamido) propyl)-4-hydroxy-5-(2-hydroxyacetamido)-2-((6-(prop-2-yn-1-yloxy) hexyl) thio) tetrahydro-2H-pyran-2-carboxylic acid (Cpd. 255)

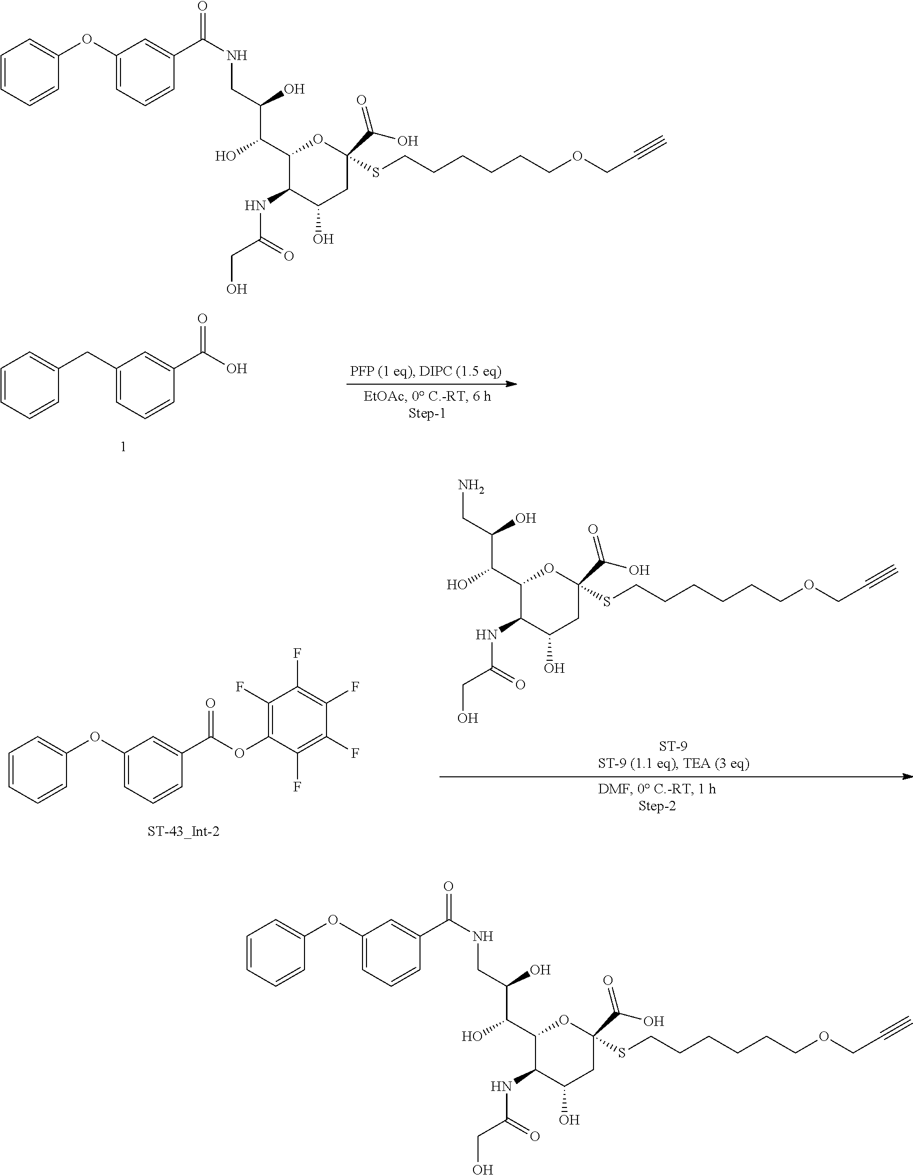

### Synthesis of perfluorophenyl 3-phenoxybenzoate (Cpd. 255_Int-2)

To a stirred solution of 3-phenoxybenzoic acid (1) (5 g, 23.34 mmol) and penta-fluoro phenol (4.30 g, 23.34 mmol) in dry ethyl acetate (50 mL) was added N, N′-di-isopropyl carbodiimide (4.42 g, 35 mmol) dropwise at 0° C. The reaction mixture was stirred at room temperature for 6 h. After completion, the reaction mixture was filtered, filtrate was concentrated and the crude residue obtained was purified by silica gel column chromatography using ethyl acetate and hexane as eluents to afford perfluorophenyl 3-phenoxybenzoate (Cpd. 255_Int-2) as off white solid, Yield-3 g, 34%. ¹H NMR (400 MHz, DMSO) δ 7.95 (d, J=7.7 Hz, 1H), 7.73-7.62 (m, 2H), 7.53-7.39 (m, 3H), 7.23 (t, J=7.4 Hz, 1H), 7.14 (dd, J=8.4, 2.4 Hz, 2H).

### Synthesis of (2S,4S,5R,6R)-6-((1R,2R)-1,2-dihydroxy-3-(3-phenoxybenzamido) propyl)-4-hydroxy-5-(2-hydroxyacetamido)-2-((6-(prop-2-yn-1-yloxy) hexyl) thio) tetrahydro-2H-pyran-2-carboxylic acid (Cpd. 255)

To a stirred solution of (2S,4S,5R,6R)-6-((1R,2R)-3-amino-1,2-dihydroxypropyl)-4-hydroxy-5-(2-hydroxyacetamido)-2-((6-(prop-2-yn-1-yloxy)hexyl)thio)tetrahydro-2H-pyran-2-carboxylic acid (ST-9) (100 mg, 0.21 mmol) in N,N-Dimethyl formamide (1 mL) were added perfluorophenyl 3-phenoxybenzoate (ST-43_Int-2) (87 mg, 0.23 mmol) and triethylamine (63 mg, 0.626 mmol) at 0° C. The resulting reaction mixture was stirred at room temperature for 1 h. After completion, the reaction mixture was purified by Prep-HPLC purification by using acetonitrile and water (+0.05%, TFA) as eluents to afford the desired product (2S,4S,5R,6R)-6-((1R,2R)-1,2-dihydroxy-3-(3-phenoxybenzamido) propyl)-4-hydroxy-5-(2-hydroxyacetamido)-2-((6-(prop-2-yn-1-yloxy) hexyl) thio) tetrahydro-2H-pyran-2-carboxylic acid (Cpd. 255) as off white solid, Yield: 70 mg, 50%; LCMS (ESI±) m/z 675.30 [M+H]⁺. ¹H NMR (400 MHz, MeOD) δ 7.58 (dt, J=7.8, 1.2 Hz, 1H), 7.52-7.43 (m, 2H), 7.43-7.36 (m, 2H), 7.21-7.12 (m, 2H), 7.08-7.00 (m, 2H), 4.09 (d, J=2.4 Hz, 2H), 4.02 (s, 2H), 4.01-3.96 (m, 1H), 3.94-3.84 (m, 2H), 3.81 (dd, J=13.8, 3.4 Hz, 1H), 3.70-3.63 (m, 1H), 3.54-3.48 (m, 1H), 3.47-3.41 (m, 3H), 3.37 (s, 1H), 2.85-2.78 (m, 2H), 2.78-2.73 (m, 1H), 2.64 (ddd, J=12.7, 8.3, 6.8 Hz, 1H), 1.86-1.75 (m, 1H), 1.60 (dt, J=14.5, 7.4 Hz, 2H), 1.51 (p, J=6.7 Hz, 2H), 1.35 (qd, J=10.7, 5.3 Hz, 4H).

### (2R,4S,5R,6R)-5-acetamido-6-((1R,2R)-3-(2-(4-chlorophenyl) acetamido)-1,2-dihydroxypropyl)-2-((2,18-dioxo-6,9,12,15,22,25-hexaoxa-3,19-diazaheptacosan-27-yl) oxy)-4-hydroxytetrahydro-2H-pyran-2-carboxylic acid (Cpd. 274)

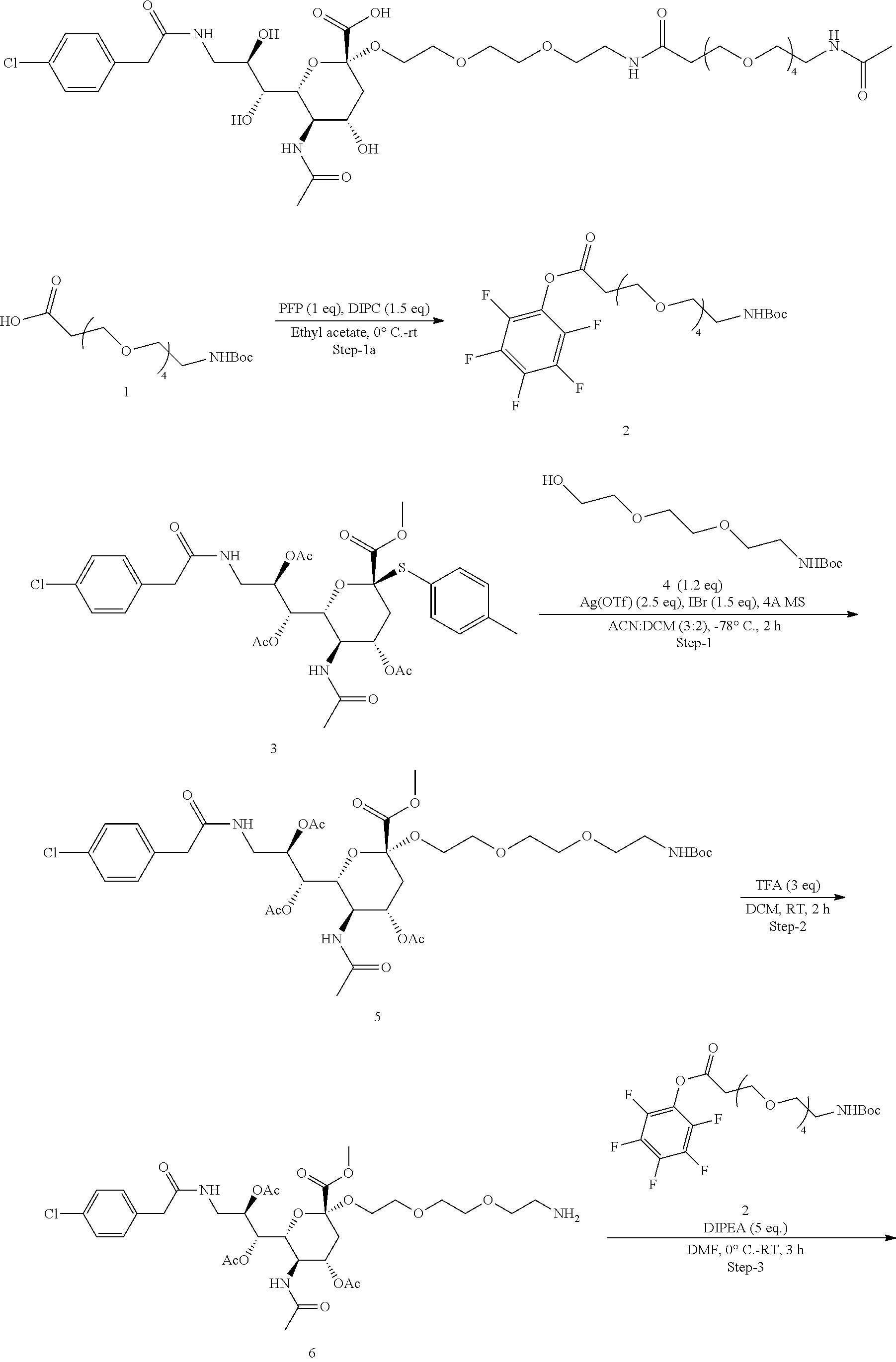

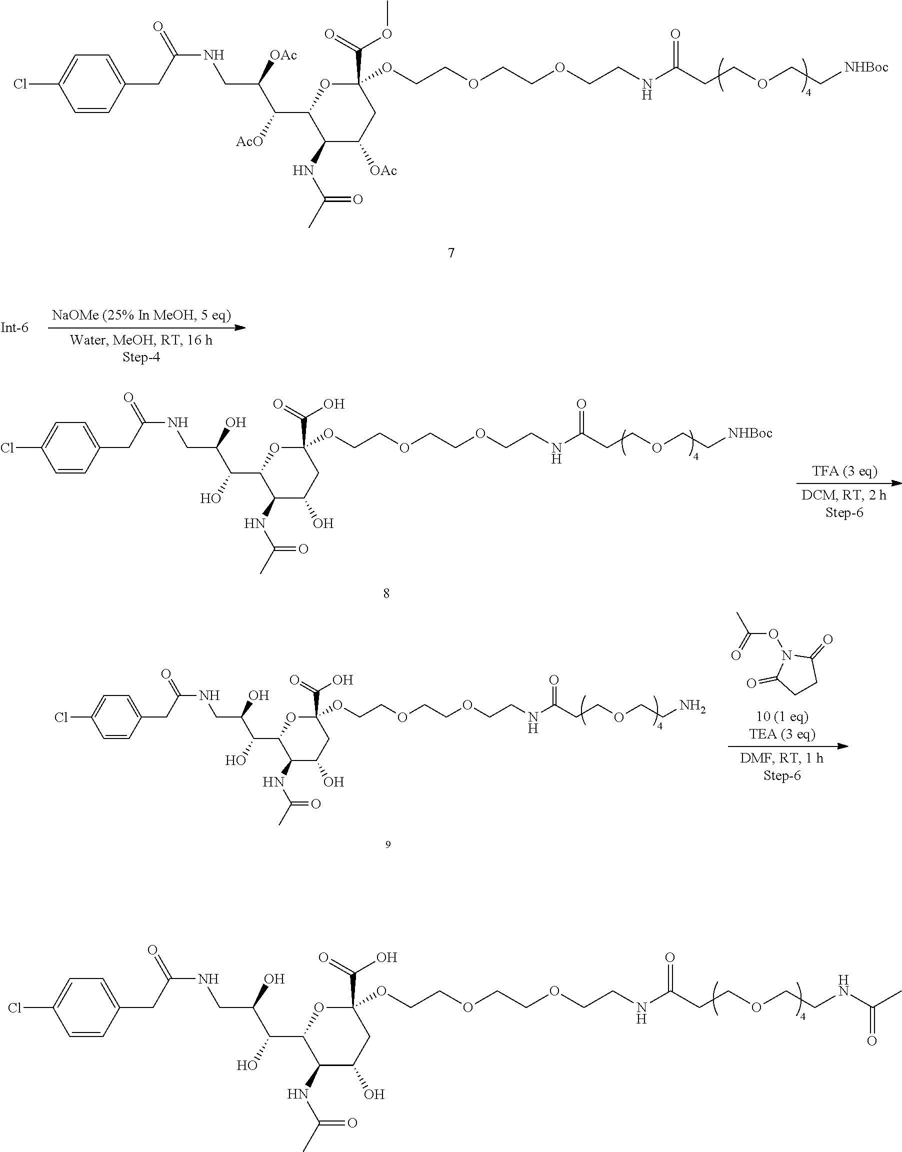

### Synthesis of perfluorophenyl 2,2-dimethyl-4-oxo-3,8,11,14,17-pentaoxa-5-azaicosan-20-oate (2)

To a stirred solution of 2,2-dimethyl-4-oxo-3,8,11,14,17-pentaoxa-5-azaicosan-20-oic acid (1) (5 g, 13.6 mmol) and penta-fluoro phenol (2.5 g, 13.6 mmol) in ethyl acetate (50 mL) was added di-iso-propyl carbodimide (2.58 g, 20.4 mmol) dropwise at 0° C. and the reaction mixture was stirred at room temperature for 6 h. After completion, the reaction mixture was filtered, filtrate was concentrated, the crude residue was purified by Silica gel column chromatography using ethyl acetate and hexane as eluents to afford perfluorophenyl 2,2-dimethyl-4-oxo-3,8,11,14,17-pentaoxa -5-azaicosan-20-oate (2) as off-white solid, Yield: 5 g, 69.44%. LCMS (ESI±) m/z 533.65 [M+H]⁺.

### Synthesis of (1R,2R)-1-((2R,3R,4S,6R)-3-acetamido-4-acetoxy-6-((2,2-dimethyl-4-oxo-3,8,11-trioxa -5-azatridecan-13-yl)oxy)-6-(methoxycarbonyl)tetrahydro-2H-pyran-2-yl)-3-(2-(4-chlorophenyl) acetamido)propane-1,2-diyl diacetate (5)

To a stirred solution of (1R,2R)-1-((2R,3R,4S,6R)-3-acetamido-4-acetoxy-6-(methoxycarbonyl) -6-(p-tolylthio)tetrahydro-2H-pyran-2-yl)-3-(2-(4-chlorophenyl)acetamido)propane-1,2-diyl diacetate (3) (2.5 g, 3.54 mmol), tert-butyl (2-(2-(2-hydroxyethoxy)ethoxy)ethyl)carbamate (4) (1.06 g, 4.24 mmol), Silver (1+) trifluoro methane sulfonate (AgOTf) (2.26 g, 8.85 mmol) in anhydrous dichloromethane (20 mL) and anhydrous acetonitrile (30 mL) was added activated 4 Å powdered molecular sieves (8 g) and the reaction mixture was stirred at room temperature for 1 h under nitrogen. The reaction mixture was cooled to −78° C., iodine monobromide (1.09 g, 5.31 mmol) dissolved in dichloromethane (8 mL) was added dropwise and the resulting mixture was stirred at the same temperature for 2 h. After completion, the reaction mixture was quenched with triethylamine (10 mL) and stirred at the room temperature. The reaction mixture was filtered, the filtrate was washed with 10% aqueous sodium thiosulfate solution, dried over sodium sulfate, filtered, concentrated under reduced pressure. The crude residue was purified by C18 reverse phase column chromatography using water and acetonitrile (+0.05% TFA) as eluents to afford the desired product (1R,2R)-1-((2R,3R,4S,6R)-3-acetamido-4-acetoxy-6-((2,2-dimethyl-4-oxo-3,8,11-trioxa-5-azatridecan-13-yl)oxy)-6-(methoxycarbonyl)tetrahydro-2H-pyran-2-yl)-3-(2-(4-chlorophenyl)acetamido)propane-1,2-diyl diacetate (5) as a pale yellow solid, Yield: 1 g, 34%; LCMS (ESI+) m/z 832.67 [M+H]⁺.

### Synthesis of (1R,2R)-1-((2R,3R,4S,6R)-3-acetamido-4-acetoxy-6-(2-(2-(2-aminoethoxy) ethoxy) ethoxy)-6-(methoxycarbonyl) tetrahydro-2H-pyran-2-yl)-3-(2-(4-chlorophenyl) acetamido) propane-1,2-diyl diacetate (6)

To a stirred solution of (1R,2R)-1-((2R,3R,4S,6R)-3-acetamido-4-acetoxy-6-((2,2-dimethyl-4-oxo-3,8,11-trioxa-5-azatridecan-13-yl)oxy)-6-(methoxycarbonyl)tetrahydro-2H-pyran-2-yl)-3-(2-(4-chlorophenyl)acetamido)propane-1,2-diyl diacetate (5) (1 g, 1.2 mmol) in anhydrous dichloromethane (10 mL) was added trifluoroacetic acid (1 mL) dropwise at 0° C. and the resulting reaction mixture was stirred at room temperature for 3 h. After completion, the reaction mixture was concentrated and the crude residue obtained was purified by prep-HPLC (15-80% acetonitrile and water, +0.05% TFA) to afford the desired product (1R,2R)-1-((2R,3R,4S,6R)-3-acetamido-4-acetoxy-6-(2-(2-(2-aminoethoxy)ethoxy)ethoxy)-6-(methoxycarbonyl)tetrahydro-2H-pyran-2-yl)-3-(2-(4-chlorophenyl)acetamido)propane-1,2-diyl diacetate (6) as yellow solid, Yield: 800 mg, 90%; LCMS (ESI+) m/z 732.6 [M+H]⁺.

### Synthesis of (1R,2R)-1-((2R,3R,4S,6R)-3-acetamido-4-acetoxy-6-((2,2-dimethyl-4,20-dioxo-3,8,11,14,17,24,27-heptaoxa-5,21-diazanonacosan-29-yl)oxy)-6-(methoxycarbonyl)tetrahydro-2H-pyran-2-yl)-3-(2-(4-chlorophenyl)acetamido)propane-1,2-diyl diacetate (7)

To a stirred solution of ((1R,2R)-1-((2R,3R,4S,6R)-3-acetamido-4-acetoxy-6-(2-(2-(2-aminoethoxy)ethoxy)ethoxy)-6-(methoxycarbonyl)tetrahydro-2H-pyran-2-yl)-3-(2-(4-chlorophenyl)acetamido)propane-1,2-diyl diacetate (6) (750 mg, 1.02 mmol) in DMF (8 mL) were added perfluorophenyl 2,2-dimethyl-4-oxo-3,8,11,14,17-pentaoxa-5-azaicosan-20-oate (2) (544 mg, 1.02 mmol) and DIPEA (0.8 mL, 5.1 mmol) at 0° c. The resulting reaction mixture was stirred at room temperature for 3 h. After completion, the reaction mixture was concentrated and the crude residue obtained was purified by C18 reverse phase column (1R,2R)-1-((2R,3R,4S,6R)-3-acetamido-4-acetoxy-6-((2,2-dimethyl-4,20-dioxo-3,8,11,14,17,24,27-heptaoxa-5,21-diazanonacosan-29-yl)oxy)-6-(methoxycarbonyl)tetrahydro-2H-pyran-2-yl)-3-(2-(4-chlorophenyl)acetamido)propane-1,2-diyl diacetate (7) as an yellow solid, Yield: 650 mg, 58%; LCMS (ESI+) m/z 1080.0 [M+H]⁺.

### Synthesis of (2R,4S,5R,6R)-5-acetamido-6-((1R,2R)-3-(2-(4-chlorophenyl) acetamido)-1,2-dihydroxypropyl)-2-((2,2-dimethyl-4,20-dioxo-3,8,11,14,17,24,27-heptaoxa-5,21-diazanonacosan-29-yl) oxy)-4-hydroxytetrahydro-2H-pyran-2-carboxylic acid (8)

To a stirred solution (1R,2R)-1-((2R,3R,4S,6R)-3-acetamido-4-acetoxy-6-((2,2-dimethyl-4,20-dioxo-3,8,11,14,17,24,27-heptaoxa-5,21-diazanonacosan-29-yl)oxy)-6-(methoxycarbonyl)tetrahydro-2H-pyran-2-yl)-3-(2-(4-chlorophenyl)acetamido)propane-1,2-diyl diacetate (7) (650 mg, 0.6 mmol) in methanol (6.5 mL) and water (2 mL) was added NaOMe (25% in MeOH) (0.68 mL, 3.01 mmol) dropwise at 0° C. The resulting reaction mixture was stirred at room temperature for 16 h. After completion, the reaction mixture was treated with acidic resin (Dowex 50, H⁺) to maintain pH˜6 and the suspension was filtered. The filtrate was concentrated under reduced pressure and the crude was purified by C18 reverse phase column chromatography using water and acetonitrile (+0.05% TFA) as eluents to afford the desired product (2R,4S,5R,6R)-5-acetamido-6-((1R,2R)-3-(2-(4-chlorophenyl)acetamido)-1,2-dihydroxypropyl)-2-((2,2-dimethyl-4,20-dioxo-3,8,11,14,17,24,27-heptaoxa-5,21-diazanonacosan-29-yl)oxy)-4-hydroxytetrahydro-2H-pyran-2-carboxylic acid (8) as an off white solid, Yield: 460 mg, 81%; LCMS (ESI+) m/z 9377.9 [M−H]⁻.

### Synthesis of (2R,4S,5R,6R)-5-acetamido-2-((1-amino-15-oxo-3,6,9,12,19,22-hexaoxa-16-azatetracosan-24-yl) oxy)-6-((1R,2R)-3-(2-(4-chlorophenyl) acetamido)-1,2-dihydroxypropyl)-4-hydroxytetrahydro-2H-pyran-2-carboxylic acid (9)

To a stirred solution of (2R,4S,5R,6R)-5-acetamido-6-((1R,2R)-3-(2-(4-chlorophenyl)acetamido)-1,2-dihydroxypropyl)-2-((2,2-dimethyl-4,20-dioxo-3,8,11,14,17,24,27-heptaoxa-5,21-diazanonacosan-29-yl)oxy)-4-hydroxytetrahydro-2H-pyran-2-carboxylic acid (8) (460 mg, 0.48 mmol) in anhydrous dichloromethane (4.6 mL) was added trifluoroacetic acid (0.5 mL) dropwise at 0° C. and the resulting reaction mixture was stirred at room temperature for 3 h. After completion, the reaction mixture was concentrated and the crude residue obtained was purified by prep-HPLC (15-80% acetonitrile and water, +0.05% TFA) to afford the desired product (2R,4S,5R,6R)-5-acetamido-2-((1-amino-15-oxo-3,6,9,12,19,22-hexaoxa-16-azatetracosan-24-yl)oxy)-6-((1R,2R)-3-(2-(4-chlorophenyl)acetamido)-1,2-dihydroxypropyl)-4-hydroxytetrahydro-2H-pyran-2-carboxylic acid (9) as an yellow solid, Yield: 300 mg, 73%; LCMS (ESI+) m/z 839.70 [M+H]⁺.

### Synthesis of (2R,4S,5R,6R)-5-acetamido-6-((1R,2R)-3-(2-(4-chlorophenyl) acetamido)-1,2-dihydroxypropyl)-2-((2,18-dioxo-6,9,12,15,22,25-hexaoxa-3,19-diazaheptacosan-27-yl) oxy)-4-hydroxytetrahydro-2H-pyran-2-carboxylic acid (Cpd. 274)

To a stirred solution of (2R,4S,5R,6R)-5-acetamido-2-((1-amino-15-oxo-3,6,9,12,19,22-hexaoxa-16-azatetracosan-24-yl)oxy)-6-((1R,2R)-3-(2-(4-chlorophenyl)acetamido)-1,2-dihydroxypropyl)-4-hydroxytetrahydro-2H-pyran-2-carboxylic acid (9) (100 mg, 0.12 mmol) in DMF (1 mL) and 2,5-dioxopyrrolidin-1-yl acetate (10) (18.72 mg, 0.12 mmol) was added triethylamine (0.03 ml, 0.18 mmol) at 0° C. The resulting reaction mixture was stirred at the same temperature for 1 h. After completion, the reaction mixture was purified by prep-HPLC (15-80% acetonitrile and water, +0.05% TFA) to afford the desired product (2R,4S,5R,6R)-5-acetamido-6-((1R,2R)-3-(2-(4-chlorophenyl)acetamido)-1,2-dihydroxypropyl)-2-((2,18-dioxo-6,9,12,15,22,25-hexaoxa-3,19-diazaheptacosan-27-yl)oxy)-4-hydroxytetrahydro-2H-pyran-2-carboxylic acid (Cpd. 274) as white solid, Yield: 30 mg, 28%. LCMS (ESI+) m/z 879.7 [M−H]⁻. ¹H NMR (400 MHz, MeOD) δ 7.31 (s, 4H), 3.93-3.86 (m, 2H), 3.79-3.71 (m, 4H), 3.69-3.57 (m, 20H), 3.59-3.53 (m, 6H), 3.40-3.36 (m, 6H), 3.32-3.24 (m, 1H), 2.72 (dd, J=12.8, 4 Hz, 1H), 2.47 (t, J=6 Hz, 2H), 1.99 (s, 3H), 1.96 (s, 3H), 1.76 (t, J=11.6 Hz, 1H).

### (2R,4S,5R,6R)-5-acetamido-6-((1R,2R)-3-(2-(4-chlorophenyl)acetamido)-1,2-dihydroxypropyl)-2-((28-(2,5-dioxo-2,5-dihydro-1H-pyrrol-1-yl)-10,26-dioxo-3,6,13,16,19,22-hexaoxa-9,25-diazaoctacosyl)oxy)-4-hydroxytetrahydro-2H-pyran-2-carboxylic acid (Cpd. 274M)

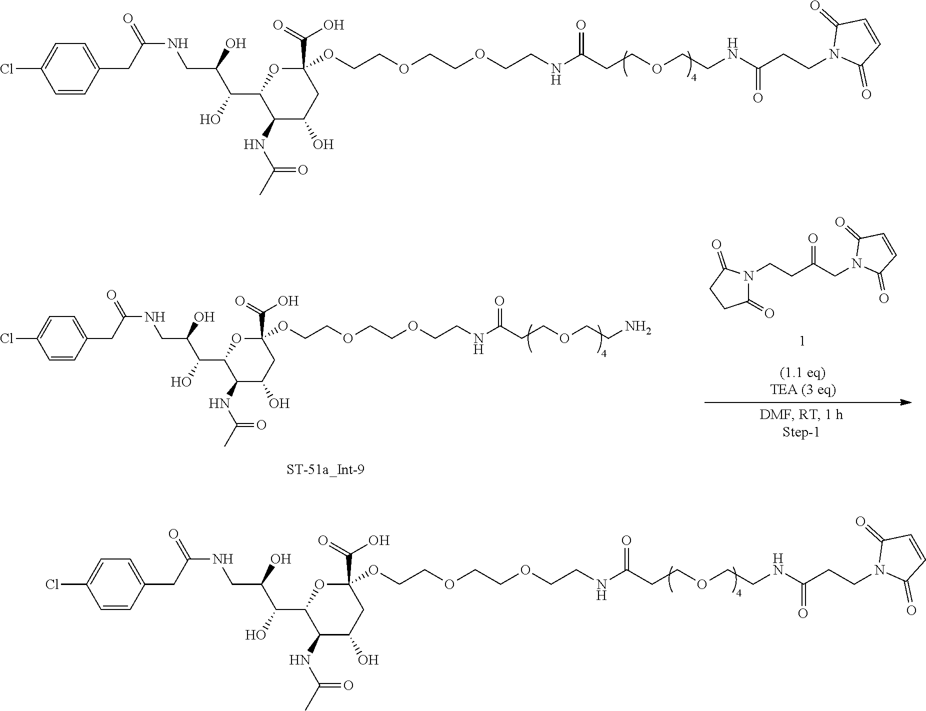

### Synthesis of (2R,4S,5R,6R)-5-acetamido-6-((1R,2R)-3-(2-(4-chlorophenyl)acetamido)-1,2-dihydroxypropyl)-2-((28-(2,5-dioxo-2,5-dihydro-1H-pyrrol-1-yl)-10,26-dioxo-3,6,13,16,19,22-hexaoxa-9,25-diazaoctacosyl)oxy)-4-hydroxytetrahydro-2H-pyran-2-carboxylic acid (Cpd. 274M)

To a stirred solution of (2R,4S,5R,6R)-5-acetamido-2-((1-amino-15-oxo-3,6,9,12,19,22-hexaoxa-16-azatetracosan-24-yl)oxy)-6-((1R,2R)-3-(2-(4-chlorophenyl)acetamido)-1,2-dihydroxypropyl)-4-hydroxytetrahydro-2H-pyran-2-carboxylic acid (Cpd. 274_Int-9) (150 mg, 0.13 mmol) in DMF (1.2 mL) were added 2,5-dioxopyrrolidin-1-yl 3-(2,5-dioxo-2,5-dihydro-1H-pyrrol-1-yl)propanoate (1) (37 mg, 0.14 mmol) and triethylamine (0.06 mL, 0.39 mmol) at 0° C. The resulting reaction mixture was stirred at the same temperature for 1 h. After completion, the reaction mixture was purified by prep-HPLC (15-80% acetonitrile and water, +0.05% TFA) to afford the desired material (2R,4S,5R,6R)-5-acetamido-6-((1R,2R)-3-(2-(4-chlorophenyl)acetamido)-1,2-dihydroxypropyl)-2-((28-(2,5-dioxo-2,5-dihydro-1H-pyrrol-1-yl)-10,26-dioxo-3,6,13,16,19,22-hexaoxa-9,25-diazaoctacosyl)oxy)-4-hydroxytetrahydro-2H-pyran-2-carboxylic acid (Cpd. 274M) as a white solid, Yield: 70 mg, 39%; LCMS (ESI+) m/z 988.7 [M−H]⁻. ¹H NMR (400 MHz, MeOD) δ 7.31 (s, 4H), 6.83 (s, 2H), 3.93-3.88 (m, 2H), 3.80-3.72 (m, 6H), 3.69-3.60 (m, 22H), 3.57-3.54 (m, 4H), 3.51 (t, J=5.6 Hz, 2H), 3.40-3.33 (m, 4H), 3.31-3.26 (m, 1H), 3.76-3.69 (m, 1H), 2.50-2.46 (m, 4H), 1.99 (m, 3H), 1.76 (t, J=11.6 Hz, 1H).

### (2R,4S,5R,6R)-5-acetamido-6-((1R,2R)-3-(2-(4-chlorophenyl)acetamido)-1,2-dihydroxypropyl)-4-hydroxy-2-((10,26,42-trioxo-46-((3aR,4R,6aS)-2-oxohexahydro-1H-thieno[3,4-d]imidazol-4-yl)-3,6,13,16,19,22,29,32,35,38-decaoxa-9,25,41-triazahexatetracontyl)oxy)tetrahydro-2H-pyran-2-carboxylic acid (Cpd. 274B)

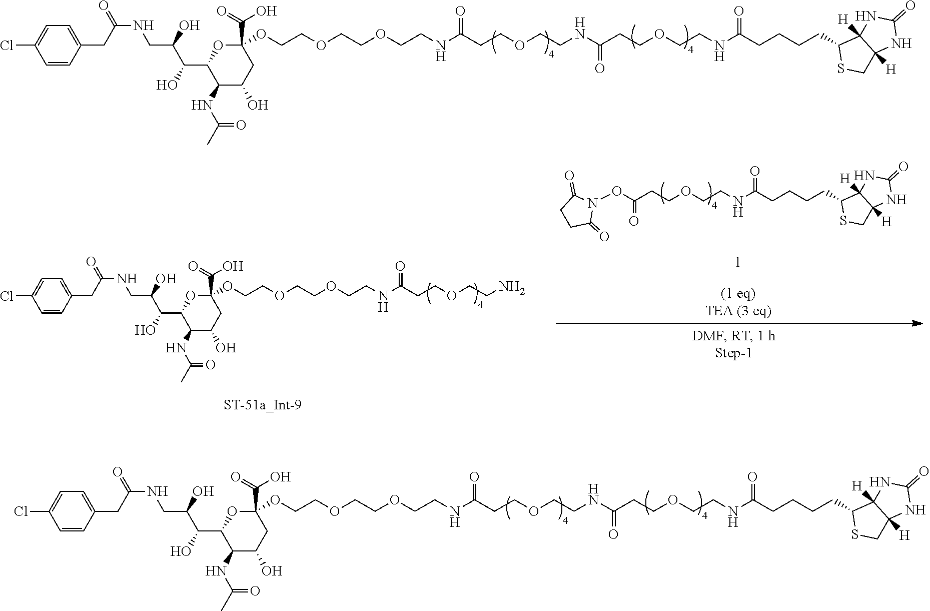

### Synthesis of (2R,4S,5R,6R)-5-acetamido-6-((1R,2R)-3-(2-(4-chlorophenyl)acetamido)-1,2-dihydroxypropyl)-4-hydroxy-2-((10,26,42-trioxo-46-((3aR,4R,6aS)-2-oxohexahydro-1H-thieno[3,4-d]imidazol-4-yl)-3,6,13,16,19,22,29,32,35,38-decaoxa-9,25,41-triazahexatetracontyl)oxy)tetrahydro-2H-pyran-2-carboxylic acid (Cpd. 274B)

To a stirred solution of (2R,4S,5R,6R)-5-acetamido-2-((1-amino-15-oxo-3,6,9,12,19,22-hexaoxa-16-azatetracosan-24-yl)oxy)-6-((1R,2R)-3-(2-(4-chlorophenyl)acetamido)-1,2-dihydroxypropyl)-4-hydroxytetrahydro-2H-pyran-2-carboxylic acid (Cpd. 274_Int-9) (50 mg, 0.05 mmol) in anhydrous N,N-dimethyl formamide (0.5 mL) were added 2,5-dioxopyrrolidin-1-yl 17-oxo-21-((3aR,4R,6aS)-2-oxohexahydro-1H-thieno[3,4-d]imidazol-4-yl)-4,7,10,13-tetraoxa-16-azahenicosanoate (1) (35 mg, 0.06 mmol) and triethylamine (0.02 ml, 0.15 mmol) at 0° C. The resulting reaction mixture was stirred at the same temperature for 2 h. After completion, the reaction mixture was purified by prep-HPLC using acetonitrile and water (+0.05%, TFA) as eluents to afford the desired product (2R,4S,5R,6R)-5-acetamido-6-((1R,2R)-3-(2-(4-chlorophenyl)acetamido)-1,2-dihydroxypropyl)-4-hydroxy-2-((10,26,42-trioxo-46-((3aR,4R,6aS)-2-oxohexahydro-1H-thieno[3,4-d]imidazol-4-yl)-3,6,13,16,19,22,29,32,35,38-decaoxa-9,25,41-triazahexatetracontyl)oxy)tetrahydro-2H-pyran-2-carboxylic acid (Cpd. 274B) as white solid, Yield: 25 mg, 32%; LCMS (ESI−) m/z 1310.8 [M−H]−. ¹H NMR (400 MHz, MeOD) δ 7.32 (s, 4H), 4.53-4.48 (m, 1H), 4.33-4.29 (m, 1H), 3.94-3.87 (m, 2H), 3.90-3.71 (m, 6H), 6.69-3.63 (m, 30H), 3.60-3.55 (m, 8H), 3.40-3.36 (m, 8H), 3.3-3.24 (m, 2H), 2.97-2.91 (m, 1H), 2.76-2.70 (m, 2H), 2.48 (t, J=6.4 Hz, 4H), 2.24 (t, J=7.2 Hz, 2H), 1.99 (s, 3H), 1.79-1.62 (m, 5H), 1.51-1.42 (m, 2H).

### (2R,4S,5R,6R)-6-((1R,2R)-3-(2-(4-(difluoro methyl) phenyl) acetamido)-1,2-dihydroxypropyl)-4-hydroxy-5-(2-hydroxyacetamido)-2-((6-(4-(15-oxo-2,5,8,11-tetraoxa-14-azahexadecyl)-1H-1,2,3-triazol-1-yl) hexyl)oxy) tetrahydro-2H-pyran-2-carboxylic acid (Cpd. 252)

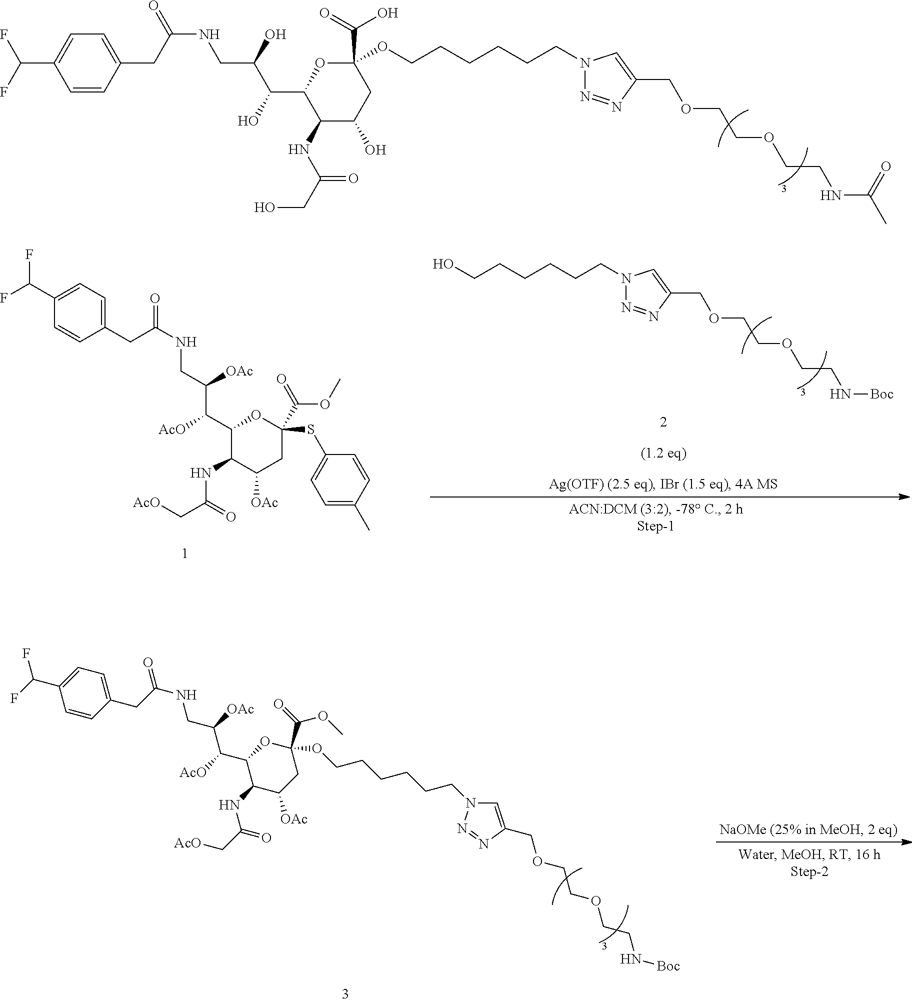

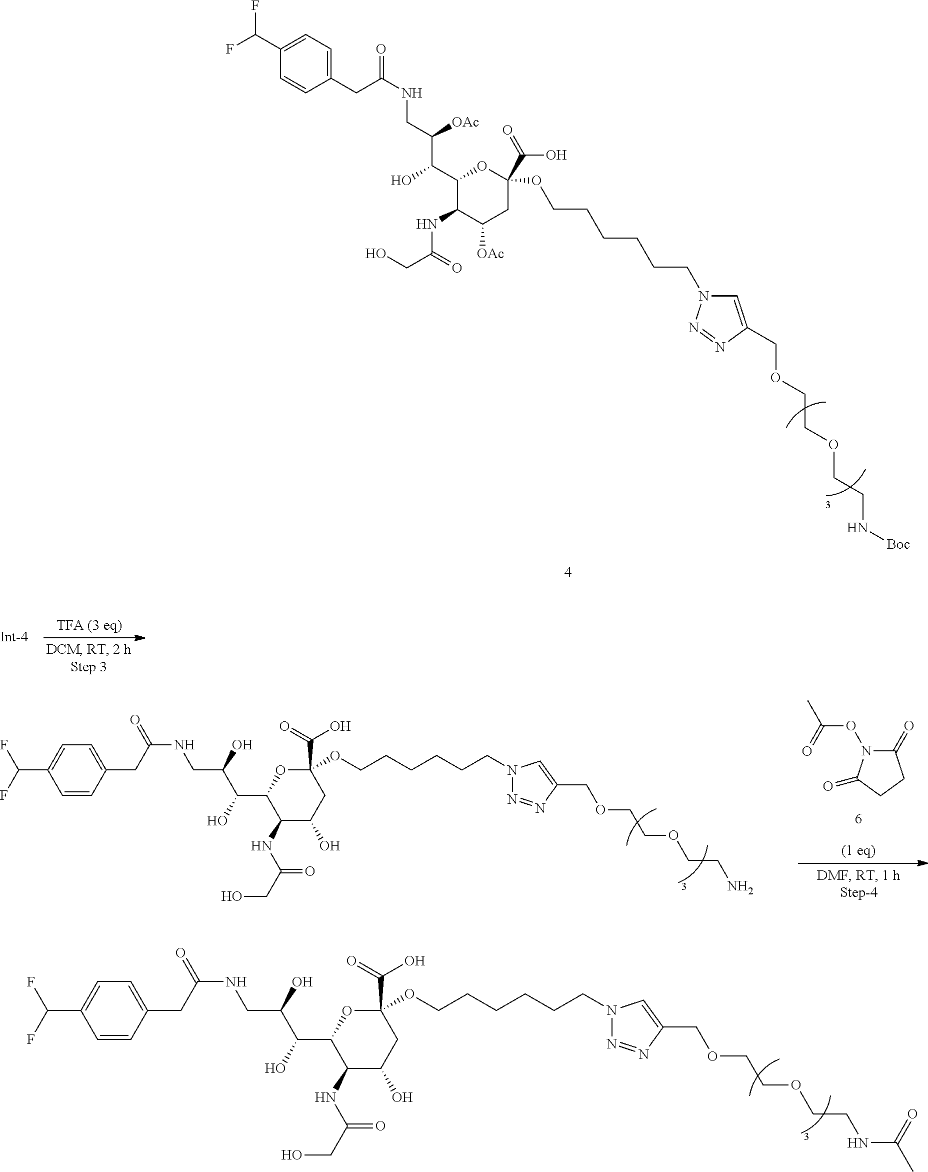

### Synthesis of (1R,2R)-1-((2R,3R,4S,6R)-4-acetoxy-3-(2-acetoxyacetamido)-6-((6-(4-(17,17-dimethyl-15-oxo-2,5,8,11,16-pentaoxa-14-azaoctadecyl)-1H-1,2,3-triazol-1-yl) hexyl)oxy)-6-(methoxycarbonyl)tetrahydro-2H-pyran-2-yl)-3-(2-(4-(difluoromethyl)phenyl)acetamido)propane-1,2-diyl diacetate (3)

To a stirred solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-(difluoromethyl)phenyl)acetamido)propane-1,2-diyl diacetate (1) (2 g, 2.56 mmol) and tert-butyl (1-(1-(6-hydroxyhexyl)-1H-1,2,3-triazol-4-yl)-2,5,8,11-tetraoxatridecan-13-yl)carbamate (2) (1.46 g, 3.07 mmol) in anhydrous dichloromethane (12 mL) and anhydrous acetonitrile (24 mL) were added Silver (1+) trifluoro methane sulfonate (AgOTf) (1.64 g, 6.40 mmol), activated 4 Å molecular sieves (5 g) and the reaction mixture was stirred at room temperature for 1 h under nitrogen. Then the reaction mixture was cooled to −78° C., iodine monobromide (0.79 g, 3.84 mmol) dissolved in dichloromethane (4 mL) was added dropwise and the resulting mixture was stirred at the same temperature for 2 h. After completion, the reaction mixture was quenched by addition of triethylamine (5 mL), filtered, the filtrate was washed with a 10% aqueous sodium thiosulfate solution, dried over sodium sulfate, filtered, concentrated under reduced pressure. The crude residue was purified by C18 reverse phase column chromatography using water and acetonitrile (+0.05% TFA) as eluents to afford the desired product (1R,2R)-1-((2R,3R,4S,6R)-4-acetoxy-3-(2-acetoxyacetamido)-6-((6-(4-(17,17-dimethyl-15-oxo-2,5,8,11,16-pentaoxa-14-azaoctadecyl)-1H-1,2,3-triazol-1-yl)hexyl)oxy)-6-(methoxycarbonyl)tetrahydro-2H-pyran-2-yl)-3-(2-(4-(difluoromethyl)phenyl)acetamido)propane-1,2-diyl diacetate (3) as a pale yellow solid, Yield: 1.5 g, 52%; MS (ESI+) m/z 1131.8 [M+H]⁺.

### Synthesis of (2R,4S,5R,6R)-6-((1R,2R)-3-(2-(4-(difluoromethyl)phenyl)acetamido)-1,2-dihydroxypropyl)-2-((6-(4-(17,17-dimethyl-15-oxo-2,5,8,11,16-pentaoxa-14-azaoctadecyl)-1H-1,2,3-triazol-1-yl)hexyl)oxy)-4-hydroxy-5-(2-hydroxyacetamido)tetrahydro-2H-pyran-2-carboxylic acid (4)

To a stirred solution of (1R,2R)-1-((2R,3R,4S,6R)-4-acetoxy-3-(2-acetoxyacetamido)-6-((6-(4-(17,17-dimethyl-15-oxo-2,5,8,11,16-pentaoxa-14-azaoctadecyl)-1H-1,2,3-triazol-1-yl)hexyl)oxy)-6-(methoxycarbonyl)tetrahydro-2H-pyran-2-yl)-3-(2-(4-(difluoromethyl)phenyl)acetamido)propane-1,2-diyl diacetate (3) (1.5 g, 1.32 mmol) in methanol (13.5 mL) and H₂O (1.5 mL) was added sodium methoxide (25% in methanol) (0.14 g, 2.64 mmol) dropwise at 0° C. The resulting reaction mixture was stirred at room temperature for 16 h. After completion, the reaction mixture was treated with acidic resin (Dowex 50, H⁺) to pH˜6 and the suspension was filtered, the filtrate was concentrated under reduced pressure and the crude was purified by C18 reverse phase column chromatography using acetonitrile and water (+0.05% TFA) as eluents to afford the desired product (2R,4S,5R,6R)-6-((1R,2R)-3-(2-(4-(difluoromethyl)phenyl)acetamido)-1,2-dihydroxypropyl)-2-((6-(4-(17,17-dimethyl-15-oxo-2,5,8,11,16-pentaoxa-14-azaoctadecyl)-1H-1,2,3-triazol-1-yl)hexyl)oxy)-4-hydroxy-5-(2-hydroxyacetamido)tetrahydro-2H-pyran-2-carboxylic acid (4) as pale-yellow solid, Yield: 1.1 g, 87%; LCMS (ESI+) m/z 950 [M+H]⁺.

### Synthesis of (2R,4S,5R,6R)-2-((6-(4-(13-amino-2,5,8,11-tetraoxatridecyl)-1H-1,2,3-triazol-1-yl)hexyl)oxy)-6-((1R,2R)-3-(2-(4-(difluoromethyl)phenyl)acetamido)-1,2-dihydroxypropyl)-4-hydroxy-5-(2-hydroxyacetamido)tetrahydro-2H-pyran-2-carboxylic acid (5)

To a stirred solution of (2R,4S,5R,6R)-6-((1R,2R)-3-(2-(4-(difluoromethyl)phenyl)acetamido)-1,2-dihydroxypropyl)-2-((6-(4-(17,17-dimethyl-15-oxo-2,5,8,11,16-pentaoxa-14-azaoctadecyl)-1H-1,2,3-triazol-1-yl)hexyl)oxy)-4-hydroxy-5-(2-hydroxyacetamido)tetrahydro-2H-pyran-2-carboxylic acid (4) (1.1 g, 1.15 mmol) in anhydrous dichloromethane (11 mL) was added trifluoroacetic acid (1 mL) dropwise at 0° C. and the resulting reaction mixture was stirred at room temperature for 3 h. After completion, the solvent was concentrated and the crude was suspended in methanol and neutralized with tetra-alkylammonium carbonate, the polymer-bound resin. The resulting mixture was filtered and the filtrate was concentrated to afford the desired product (2R,4S,5R,6R)-2-((6-(4-(13-amino-2,5,8,11-tetraoxatridecyl)-1H-1,2,3-triazol-1-yl)hexyl)oxy)-6-((1R,2R)-3-(2-(4-(difluoromethyl)phenyl)acetamido)-1,2-dihydroxypropyl)-4-hydroxy-5-(2-hydroxyacetamido)tetrahydro-2H-pyran-2-carboxylic acid (5) as yellow solid, Yield: 440 mg, 44%; LCMS (ESI+) m/z 849.7 [M+H]⁺.

### Synthesis of (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((6-(4-(15-oxo-2,5,8,11-tetraoxa-14-azahexadecyl)-1H-1,2,3-triazol-1-yl)hexyl)oxy)tetrahydro-2H-pyran-2-carboxylic acid (252)

To a stirred solution of (2R,4S,5R,6R)-2-((6-(4-(13-amino-2,5,8,11-tetraoxatridecyl)-1H-1,2,3-triazol-1-yl)hexyl)oxy)-6-((1R,2R)-3-(2-(4-(difluoromethyl)phenyl)acetamido)-1,2-dihydroxypropyl)-4-hydroxy-5-(2-hydroxyacetamido)tetrahydro-2H-pyran-2-carboxylic acid (5) (10 mg, 0.12 mmol) in DMF (1 mL) was added 2,5-dioxopyrrolidin-1-yl acetate (6) (0.02 g, 0.117 mmol) at 0° C. and the resulting reaction mixture was stirred at the same temperature for 1 h. After completion, the reaction mixture was purified by C18 prep-HPLC using acetonitrile and water (+0.1% TFA) to afford the desired product (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((6-(4-(15-oxo-2,5,8,11-tetraoxa-14-azahexadecyl)-1H-1,2,3-triazol-1-yl)hexyl)oxy)tetrahydro-2H-pyran-2-carboxylic acid (252) as off-white solid, Yield: 30 mg, 33.3%; LCMS (ESI+) m/z 891.4 [M+H]⁺. ¹H NMR (400 MHz, MeOD) δ 8.00 (s, 1H), 7.91 (d, J=7.8 Hz, 1H), 7.47 (dd, J=20.0, 8.0 Hz, 4H), 6.91 (t, J=56 Hz, 1H), 4.66 (s, 2H), 4.43-4.40 (m, 2H), 4.66 (s, 2H), 4.04 (s, 2H), 4.04 (s, 2H), 3.91-3.71 (m, 10H), 3.69-3.62 (m, 12H), 3.57-3.27 (m, 5H), 2.73 (dd, J=28, 12 Hz, 1H), 1.96 (s, 3H), 1.92 (t, J=8 Hz, 2H), 1.58-1.55 (m, 1H), 1.43-1.33 (m, 6H).

### (2R,4S,5R,6R)-6-((1R,2R)-3-(2-(4-(difluoromethyl)phenyl)acetamido)-1,2-dihydroxypropyl)-2-((6-(4-(17-(2,5-dioxo-2,5-dihydro-1H-pyrrol-1-yl)-15-oxo-2,5,8,11-tetraoxa-14-azaheptadecyl)-1H-1,2,3-triazol-1-yl)hexyl)oxy)-4-hydroxy-5-(2-hydroxyacetamido)tetrahydro-2H-pyran-2-carboxylic acid(Cpd. 252M)

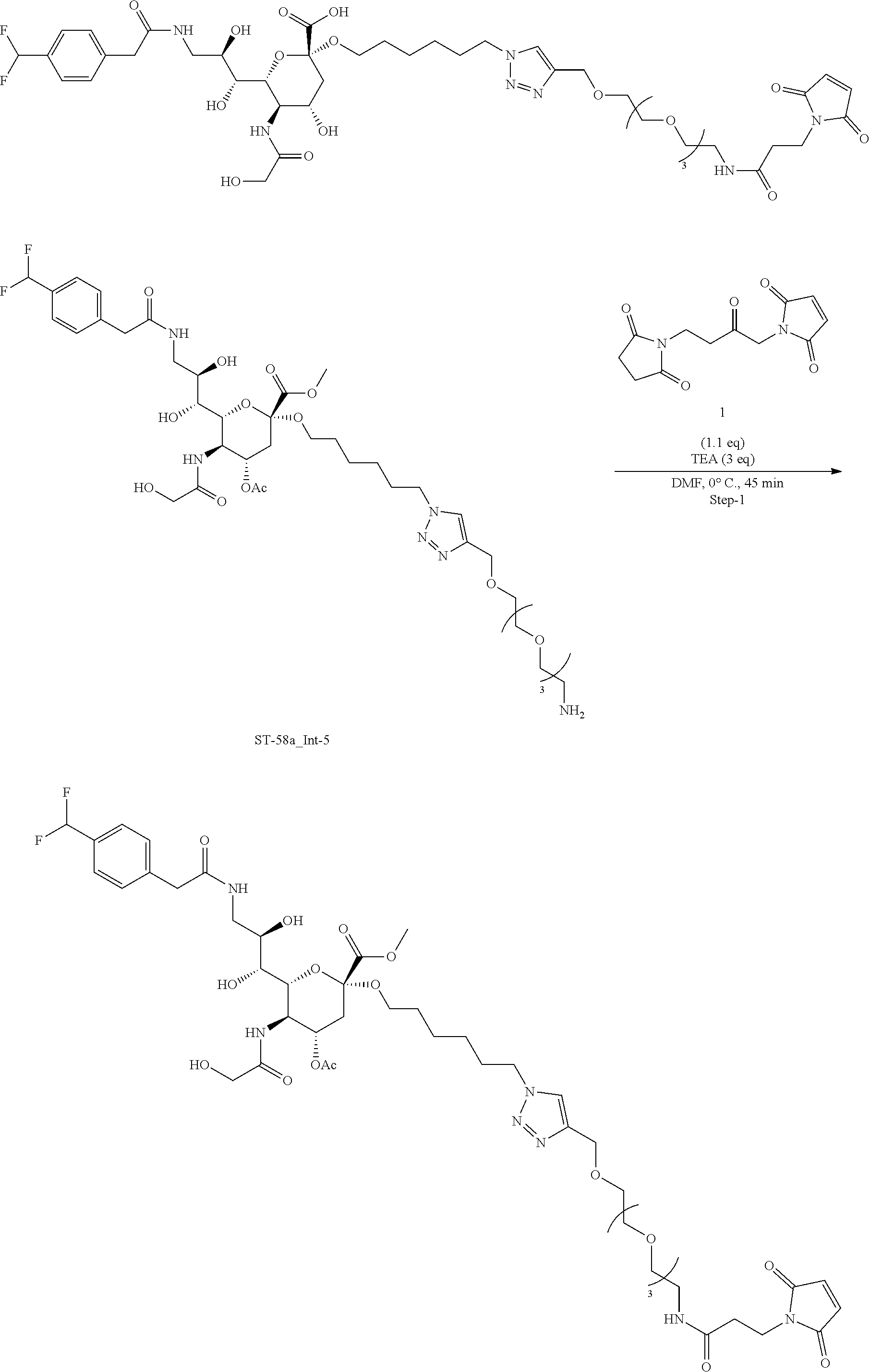

### Synthesis of (2R,4S,5R,6R)-6-((1R,2R)-3-(2-(4-(difluoromethyl)phenyl)acetamido)-1,2-dihydroxypropyl)-2-((6-(4-(17-(2,5-dioxo-2,5-dihydro-1H-pyrrol-1-yl)-15-oxo-2,5,8,11-tetraoxa-14-azaheptadecyl)-1H-1,2,3-triazol-1-yl)hexyl)oxy)-4-hydroxy-5-(2-hydroxy acetamido) tetrahydro-2H-pyran-2-carboxylic acid (252M)

To a stirred solution of (2R,4S,5R,6R)-2-((6-(4-(13-amino-2,5,8,11-tetraoxatridecyl)-1H-1,2,3-triazol-1-yl)hexyl)oxy)-6-((1R,2R)-3-(2-(4-(difluoromethyl)phenyl)acetamido)-1,2-dihydroxypropyl)-4-hydroxy-5-(2-hydroxyacetamido)tetrahydro-2H-pyran-2-carboxylic acid (5) (0.150 g, 0.176 mmol) in N,N-Dimethyl formamide (1.5 mL) were added 2,5-dioxopyrrolidin-1-yl 3-(2,5-dioxo-2,5-dihydro-1H-pyrrol-1-yl)propanoate (1) (0.052 g, 0.193 mmol) and triethylamine (0.1 mL, 0.528 mmol) at 0° C. The resulting reaction mixture was stirred at 0° C. for 45 min. After completion, the reaction mixture was purified by C18 prep-HPLC using acetonitrile and water (+0.05%, TFA) as eluents to afford the desired product (2R,4S,5R,6R)-6-((1R,2R)-3-(2-(4-(difluoromethyl)phenyl)acetamido)-1,2-dihydroxypropyl)-2-((6-(4-(17-(2,5-dioxo-2,5-dihydro-1H-pyrrol-1-yl)-15-oxo-2,5,8,11-tetraoxa-14-azaheptadecyl)-1H-1,2,3-triazol-1-yl)hexyl)oxy)-4-hydroxy-5-(2-hydroxyacetamido)tetrahydro-2H-pyran-2-carboxylic acid (252M) as off-white solid, Yield: 0.053 g, 30%; LCMS (ESI+) m/z 1001.4 [M+H]⁺. ¹H NMR (400 MHz, MeOD): δ 8.01 (s, 1H), 7.91 (d, J=8 Hz, 1H), 7.47 (dd, J=20.0, 8.0 Hz, 4H), 6.91 (t, J=56 Hz, 1H), 4.66 (s, 2H), 4.42 (t, J=8 Hz, 2H), 4.04 (s, 2H), 3.81-3.61 (m, 21H), 3.53-3.31 (m, 8H), 2.73 (dd, J=28, 12 Hz, 1H), 2.50-2.47 (m, 2H), 1.92 (t, J=8 Hz, 2H), 1.75-1.35 (m, 8H).

### (2R,4S,5R,6R)-6-((1R,2R)-3-(2-(4-(difluoromethyl)phenyl)acetamido)-1,2-dihydroxypropyl)-2-((6-(4-(15,31-dioxo-35-((3aR,4R,6aS)-2-oxohexahydro-1H-thieno[3,4-d]imidazol-4-yl)-2,5,8,11,18,21,24,27-octaoxa-14,30-diazapentatriacontyl)-1H-1,2,3-triazol-1-yl)hexyl)oxy)-4-hydroxy-5-(2-hydroxyacetamido)tetrahydro-2H-pyran-2-carboxylic acid (Cpd. 252B)

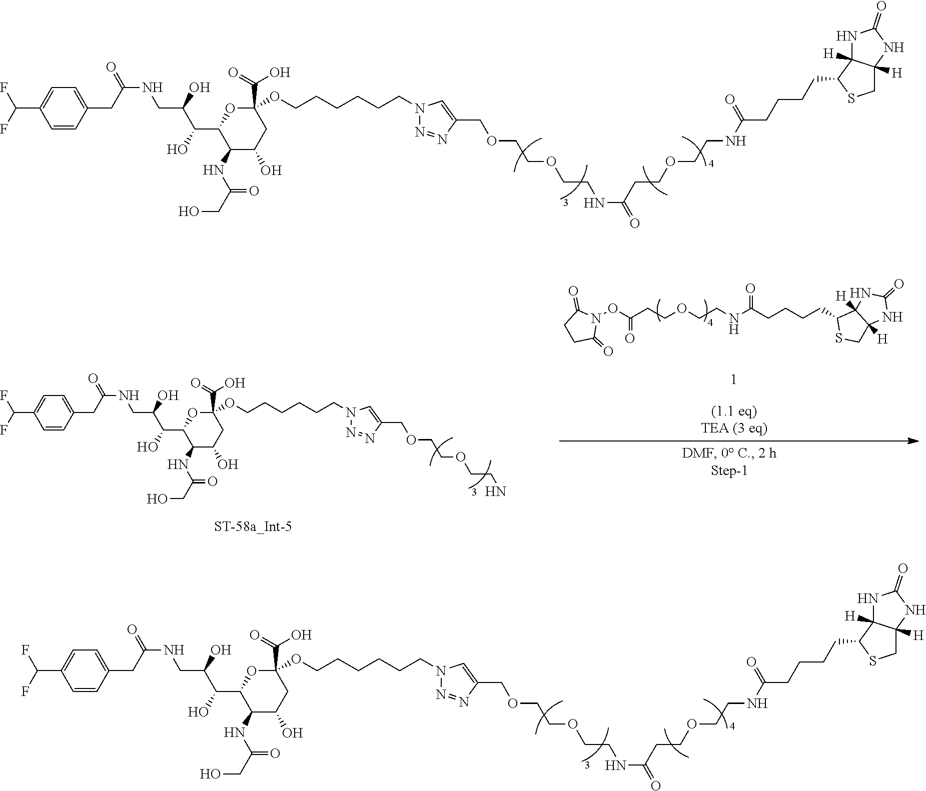

### Synthesis of (2R,4S,5R,6R)-6-((1R,2R)-3-(2-(4-(difluoromethyl)phenyl)acetamido)-1,2-dihydroxypropyl)-2-((6-(4-(15,31-dioxo-35-((3aR,4R,6aS)-2-oxohexahydro-1H-thieno[3,4-d]imidazol-4-yl)-2,5,8,11,18,21,24,27-octaoxa-14,30-diazapentatriacontyl)-1H-1,2,3-triazol-1-yl)hexyl)oxy)-4-hydroxy-5-(2-hydroxyacetamido)tetrahydro-2H-pyran-2-carboxylic acid (252B)

To a stirred solution of (2R,4S,5R,6R)-2-((6-(4-(13-amino-2,5,8,11-tetraoxatridecyl)-1H-1,2,3-triazol-1-yl)hexyl)oxy)-6-((1R,2R)-3-(2-(4-(difluoromethyl)phenyl)acetamido)-1,2-dihydroxypropyl)-4-hydroxy-5-(2-hydroxyacetamido)tetrahydro-2H-pyran-2-carboxylic acid (5) (0.050 g, 0.058 mmol) in N,N-Dimethyl formamide (0.5 mL) were added 2,5-dioxopyrrolidin-1-yl 17-oxo-21-((3aR,4R,6aS)-2-oxohexahydro-1H-thieno[3,4-d]imidazol-4-yl)-4,7,10,13-tetraoxa-16-azahenicosanoate (1) (0.037 g, 0.064 mmol) and triethylamine (0.03 mL, 0.174 mmol) at 0° C. The resulting reaction mixture was stirred at 0° C. for 2 h. After completion, the reaction mixture was purified by C18 prep-HPLC using acetonitrile and water (+0.05%, TFA) as eluents to afford the desired product (2R,4S,5R,6R)-6-((1R,2R)-3-(2-(4-(difluoromethyl)phenyl)acetamido)-1,2-dihydroxypropyl)-2-((6-(4-(15,31-dioxo-35-((3aR,4R,6aS)-2-oxohexahydro-1H-thieno[3,4-d]imidazol-4-yl)-2,5,8,11,18,21,24,27-octaoxa-14,30-diazapentatriacontyl)-1H-1,2,3-triazol-1-yl)hexyl)oxy)-4-hydroxy-5-(2-hydroxyacetamido)tetrahydro-2H-pyran-2-carboxylic acid (252B) as off-white solid, Yield: 20 mg, 26%; LCMS (ESI+) m/z 1323 [M+H]⁺. ¹H NMR (400 MHz, MeOD): δ 7.99 (s, 1H), 7.91 (d, J=8 Hz, 1H), 7.46 (dd, J=21.2, 7.6 Hz, 4H), 6.91 (t, J=56 Hz, 1H), 4.69-4.41 (m, 1H), 4.32 (t, J=8 Hz, 2H), 4.32-4.41 (m, 1H), 4.07 (s, 2H). 3.90-3.62 (m, 5H) 3.72-3.62 (m, 25H). 3.55-3.53 (m, 5H) 3.42-3.35 (m, 4H), 3.31-3.20 (m, 2H), 2.92-2.89 (m, 1H), 2.71 (t, J=8 Hz, 2H) 2.46 (t, J=8 Hz, 2H), 2.23 (t, J=8 Hz, 2H), 1.90 (t, J=6.4 Hz, 2H), 1.73-1.52 (m, 8H), 1.46-1.31 (m, 7H).

### (2R,4S,5R,6R)-6-((1R,2R)-3-(2-(4-(difluoro methyl) phenyl)acetamido)-1,2-dihydroxypropyl)-4-hydroxy-5-(2-hydroxyacetamido)-2-((8-(4-(15-oxo-2,5,8,11-tetraoxa-14-azahexadecyl)-1H-1,2,3-triazol-1-yl)octyl)oxy)tetrahydro-2H-pyran-2-carboxylic acid (Cpd. 259)

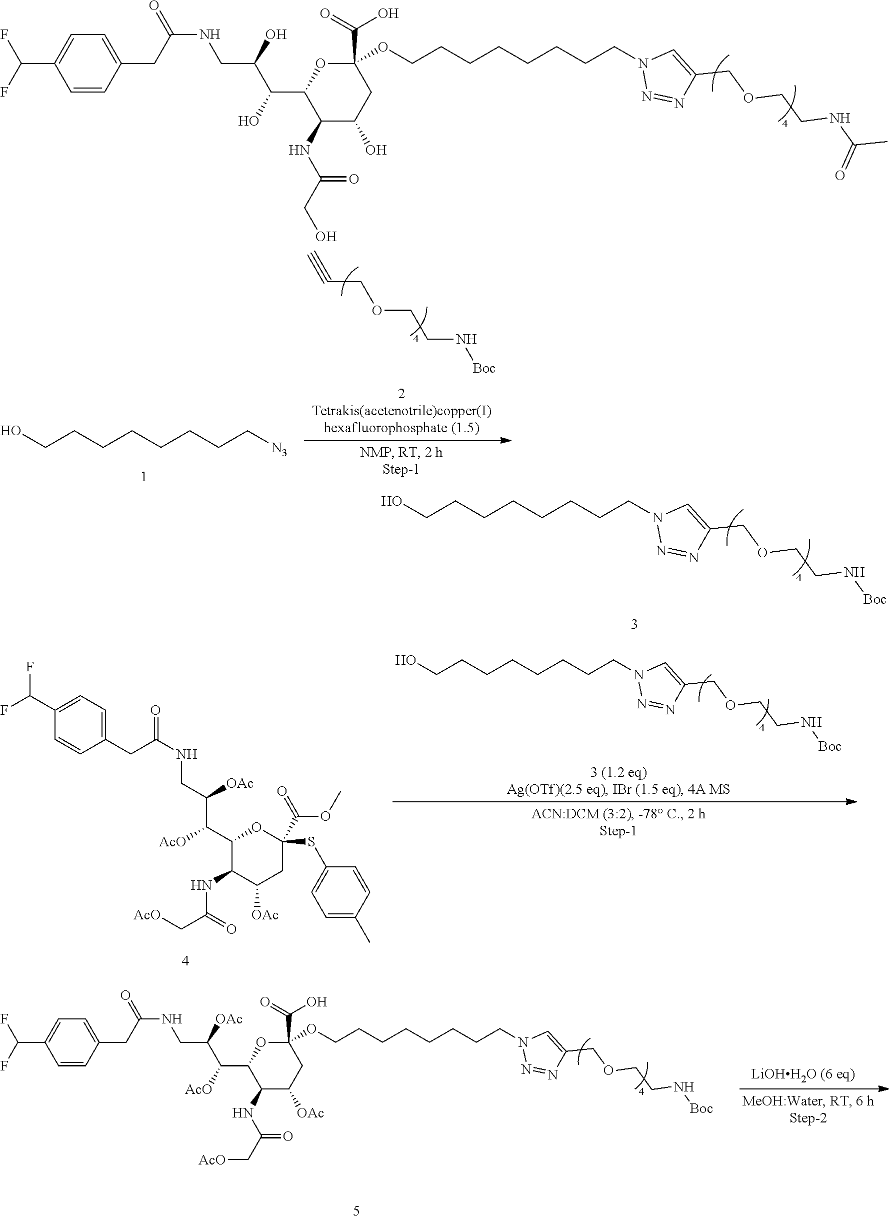

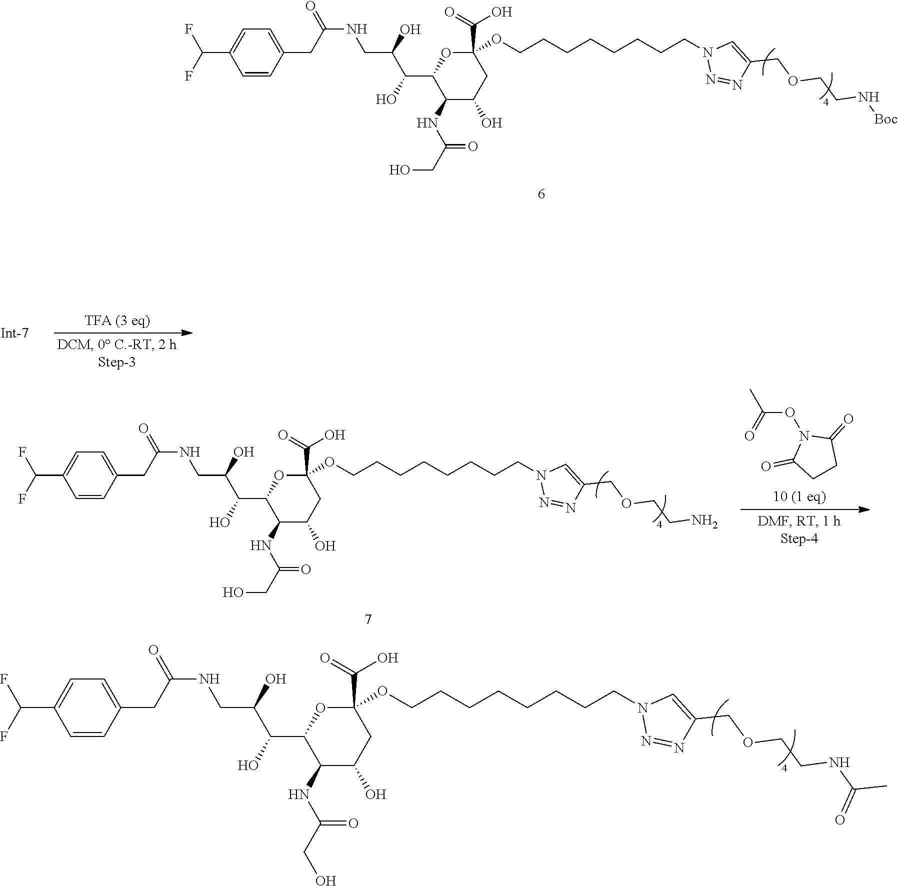

### Synthesis of tert-butyl (1-(1-(8-hydroxyoctyl)-1H-1,2,3-triazol-4-yl)-2,5,8,11-tetraoxatridecan-13-yl) carbamate (3)

To a stirred solution of 8-azidooctan-1-ol (1) (3 g, 17.52 mmol) and tert-butyl (3,6,9,12-tetraoxapentadec-14-yn-1-yl) carbamate (2) (5.81 g, 17.52 mmol.) in NMP (20 mL) was added Tetrakis(acetonitrile)copper(I)hexafluorophosphate (9.7 g, 26.3 mmol, 1.5 eq.), then the reaction mixture was stirred at room temperature for 2 h. After completion, the reaction mixture was concentrated under reduced pressure and the crude residue obtained was purified by reverse phase prep-HPLC using (15-80% acetonitrile in water, +0.05% TFA) to get the desired compound tert-butyl (1-(1-(8-hydroxyoctyl)-1H-1,2,3-triazol-4-yl)-2,5,8,11-tetraoxatridecan-13-yl) carbamate (3) as colorless semi solid, Yield: 4.2 g, 47%; LCMS (ESI+) m/z 503.5 [M+H]⁺.

### Synthesis of (1R,2R)-1-((2R,3R,4S,6R)-4-acetoxy-3-(2-acetoxyacetamido)-6-((6-(4-(17,17-dimethyl-15-oxo-2,5,8,11,16-pentaoxa-14-azaoctadecyl)-1H-1, 2, 3-triazol-1-yl) hexyl)oxy)-6-(methoxycarbonyl)tetrahydro-2H-pyran-2-yl)-3-(2-(4-(difluoromethyl)phenyl)acetamido) propane-1,2-diyl diacetate (5)

To a stirred solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-(difluoromethyl)phenyl) acetamido)propane-1,2-diyl diacetate (4) (2 g, 2.56 mmol), tert-butyl (1-(1-(8-hydroxyoctyl)-1H-1,2,3-triazol-4-yl)-2,5,8,11-tetraoxatridecan-13-yl)carbamate (3) (1.82 g, 3.84 mmol), Silver (1+) trifluoro methane sulfonate (AgOTf) (1.64 g, 6.40 mmol) in anhydrous dichloromethane (12 mL) and anhydrous acetonitrile (24 mL) was added activated 4 Å powdered molecular sieves (5 g). The reaction mixture was stirred at room temperature for 1 h under nitrogen atmosphere. The solution was cooled to −78° C., iodine monobromide (794 mg, 3.84 mmol) dissolved in dichloromethane (4 mL) was added dropwise and the resulting mixture was stirred at same temperature +. After completion, the reaction mixture was quenched by addition of triethylamine (5 mL) at the same temperature and warmed to room temperature. The reaction mixture was filtered, the filtrate was washed with 10% aqueous sodium thiosulfate solution, dried over sodium sulfate, filtered, concentrated under reduced pressure. The crude residue obtained was purified by C18 reverse phase column chromatography using water and acetonitrile (+0.05% TFA) as eluents to afford the desired product (1R,2R)-1-((2R,3R,4S,6R)-4-acetoxy-3-(2-acetoxyacetamido)-6-((6-(4-(17,17-dimethyl-15-oxo-2,5,8,11,16-pentaoxa-14-azaoctadecyl)-1H-1,2,3-triazol-1-yl) hexyl)oxy)-6-(methoxycarbonyl)tetrahydro-2H-pyran-2-yl)-3-(2-(4-(difluoromethyl)phenyl)acetamido) propane-1,2-diyl diacetate (5) as a pale yellow solid, Yield-1.5 g, 51.72%; LCMS (ESI) m/z 1160.1 [M+H]⁺.

### Synthesis of (2R,4S,5R,6R)-6-((1R,2R)-3-(2-(4-(difluoromethyl)phenyl)acetamido)-1,2-dihydroxypropyl)-2-((8-(4-(17,17-dimethyl-15-oxo-2,5,8,11,16-pentaoxa-14-azaoctadecyl)-1H-1,2,3-triazol-1-yl)octyl)oxy)-4-hydroxy-5-(2-hydroxyacetamido)tetrahydro-2H-pyran-2-carboxylic acid (6)

To a stirred solution of (1R,2R)-1-((2R,3R,4S,6R)-4-acetoxy-3-(2-acetoxyacetamido)-6-((8-(4-(17,17-dimethyl-15-oxo-2,5,8,11,16-pentaoxa-14-azaoctadecyl)-1H-1,2,3-triazol-1-yl)octyl)oxy)-6-(methoxycarbonyl)tetrahydro-2H-pyran-2-yl)-3-(2-(4-(difluoromethyl)phenyl)acetamido)propane-1,2-diyl diacetate (5) (1.5 g, 1.32 mmol) in methanol (9 mL) was added lithium hydroxide monohydrate (334 mg, 7.95 mmol, 6 eq.) in water (1 mL) at 0° C. and the reaction mixture was stirred at room temperature for 16 h. After completion, the reaction mixture was treated with acidic resin (Dowex 50, H⁺) to maintain pH˜6 and the suspension was filtered. The filtrate was concentrated under reduced pressure and the crude residue obtained was purified by C18 reverse phase column chromatography using water and acetonitrile (+0.05% TFA) as eluents to afford the desired product (2R,4S,5R,6R)-6-((1R,2R)-3-(2-(4-(difluoromethyl)phenyl)acetamido)-1,2-dihydroxypropyl)-2-((8-(4-(17,17-dimethyl-15-oxo-2,5,8,11,16-pentaoxa-14-azaoctadecyl)-1H-1,2,3-triazol-1-yl)octyl)oxy)-4-hydroxy-5-(2-hydroxyacetamido)tetrahydro-2H-pyran-2-carboxylic acid (6) as an yellow gummy solid, Yield: 700 mg, 55%; LCMS (ESI) m/z 978.7 [M+H]⁺.

### Synthesis of (2R,4S,5R,6R)-2-((8-(4-(13-amino-2,5,8,11-tetraoxatridecyl)-1H-1,2,3-triazol-1-yl) octyl) oxy)-6-((1R,2R)-3-(2-(4-(difluoromethyl) phenyl) acetamido)-1,2-dihydroxypropyl)-4-hydroxy-5-(2-hydroxyacetamido) tetrahydro-2H-pyran-2-carboxylic acid (7)

To a stirred solution of (2R,4S,5R,6R)-6-((1R,2R)-3-(2-(4-(difluoromethyl)phenyl)acetamido)-1,2-dihydroxypropyl)-2-((8-(4-(17,17-dimethyl-15-oxo-2,5,8,11,16-pentaoxa-14-azaoctadecyl)-1H-1,2,3-triazol-1-yl)octyl)oxy)-4-hydroxy-5-(2-hydroxyacetamido)tetrahydro-2H-pyran-2-carboxylic acid (6) (700 mg, 0.71 mmol) in anhydrous dichloromethane (8 mL) was added trifluoro acetic acid (2.34 mL) dropwise at 0° C. and the resulting reaction mixture was stirred at room temperature for 3 h. After completion, the solvent was concentrated and the crude residue obtained was purified by prep-HPLC using 15-80% acetonitrile in water, +0.05% TFA as eluents to afford the desired product (2R,4S,5R,6R)-2-((8-(4-(13-amino-2,5,8,11-tetraoxatridecyl)-1H-1,2,3-triazol-1-yl)octyl)oxy)-6-((1R,2R)-3-(2-(4-(difluoromethyl)phenyl)acetamido)-1,2-dihydroxypropyl)-4-hydroxy-5-(2-hydroxyacetamido)tetrahydro-2H-pyran-2-carboxylic acid (7) as a yellow solid, Yield: 450 mg, 71%; LCMS (ESI+) m/z 877.4 [M+H]⁺.

### Synthesis of (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((8-(4-(15-oxo-2,5,8,11-tetraoxa-14-azahexadecyl)-1H-1,2,3-triazol-1-yl)octyl)oxy)tetrahydro-2H-pyran-2-carboxylic acid (259)

To a stirred solution of (2R,4S,5R,6R)-2-((8-(4-(13-amino-2,5,8,11-tetraoxatridecyl)-1H-1,2,3-triazol-1-yl)octyl)oxy)-6-((1R,2R)-3-(2-(4-(difluoromethyl)phenyl)acetamido)-1,2-dihydroxypropyl)-4-hydroxy-5-(2-hydroxyacetamido)tetrahydro-2H-pyran-2-carboxylic acid (7) (100 mg, 0.11 mmol) in DMF (0.7 mL) was added 2,5-dioxopyrrolidin-1-yl acetate (11) (17.92 mg, 0.11 mmol) at 0° C. The resulting reaction mixture was stirred at the same temperature for 1 h. After completion, the reaction mixture was purified by prep-HPLC using 15-80% acetonitrile in water with +0.05% TFA, to afford the desired product (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((8-(4-(15-oxo-2,5,8,11-tetraoxa-14-azahexadecyl)-1H-1,2,3-triazol-1-yl)octyl)oxy)tetrahydro-2H-pyran-2-carboxylic acid (259) as white solid, Yield: 85 mg, 81%; LCMS (ESI+) m/z 919.4 [M+H]⁺. ¹H NMR (400 MHz, MeOD) δ 7.99 (s, 1H), 7.46 (dd, J=22.0, 8.0 Hz, 4H), 6.75 (t, J=56 Hz, 1H), 4.65 (s, 2H), 4.42 (t, J=4 Hz, 2H), 4.03 (s, 2H), 3.90-3.64 (m, 21H), 3.54 (t, J=4 Hz, 2H), 3.41-3.40 (m, 2H), 3.36-3.32 (m, 1H), 2.74-2.70 (m, 1H), 1.95 (s, 3H), 1.93-1.91 (m, 2H), 1.74 (t, J=12.4 Hz, 1H), 1.61-1.52 (m, 2H), 1.34 (s, 9H).

### (2R,4S,5R,6R)-6-((1R,2R)-3-(2-(4-(difluoromethyl)phenyl)acetamido)-1,2-dihydroxypropyl)-2-((8-(4-(17-(2,5-dioxo-2,5-dihydro-1H-pyrrol-1-yl)-15-oxo-2,5,8,11-tetraoxa-14-azaheptadecyl)-1H-1,2,3-triazol-1-yl)octyl)oxy)-4-hydroxy-5-(2-hydroxyacetamido)tetrahydro-2H-pyran-2-carboxylic acid (Cpd. 259M)

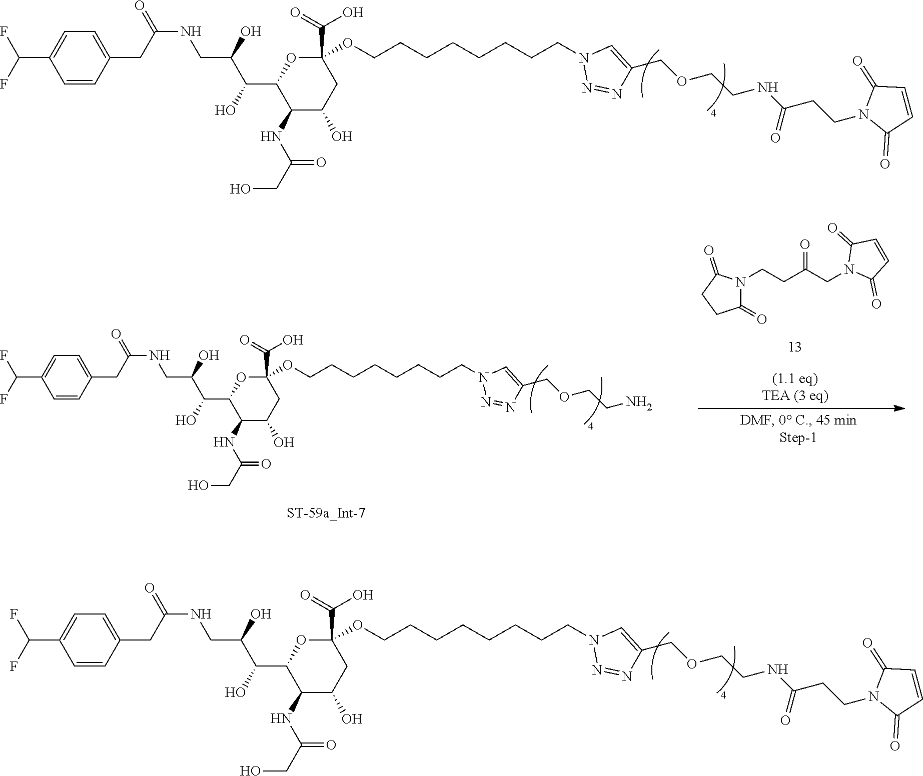

### Synthesis of (2R,4S,5R,6R)-6-((1R,2R)-3-(2-(4-(difluoromethyl)phenyl)acetamido)-1,2-dihydroxypropyl)-2-((8-(4-(17-(2,5-dioxo-2,5-dihydro-1H-pyrrol-1-yl)-15-oxo-2,5,8,11-tetraoxa-14-azaheptadecyl)-1H-1,2,3-triazol-1-yl)octyl)oxy)-4-hydroxy-5-(2-hydroxyacetamido)tetrahydro-2H-pyran-2-carboxylic acid (259M)

To a stirred solution of (2R,4S,5R,6R)-2-((8-(4-(13-amino-2,5,8,11-tetraoxatridecyl)-1H-1,2,3-triazol-1-yl)octyl)oxy)-6-((1R,2R)-3-(2-(4-(difluoromethyl)phenyl)acetamido)-1,2-dihydroxypropyl)-4-hydroxy-5-(2-hydroxyacetamido)tetrahydro-2H-pyran-2-carboxylic acid (259_Int-7) (150 mg, 0.17 mmol) in N,N-dimethyl formamide (1.5 mL) were added 2,5-dioxopyrrolidin-1-yl 3-(2,5-dioxo-2,5-dihydro-1H-pyrrol-1-yl)propanoate (1) (45.5 mg, 0.17 mmol) and triethylamine (0.1 mL, 0.51 mmol) at 0° C. The resulting reaction mixture was stirred at the same temperature for 45 min. After completion, the reaction mixture was concentrated and purified by prep-HPLC using acetonitrile and water (+0.05%, TFA) as eluents to (2R,4S,5R,6R)-6-((1R,2R)-3-(2-(4-(difluoromethyl)phenyl)acetamido)-1,2-dihydroxypropyl)-2-((8-(4-(17-(2,5-dioxo-2,5-dihydro-1H-pyrrol-1-yl)-15-oxo-2,5,8,11-tetraoxa-14-azaheptadecyl)-1H-1,2,3-triazol-1-yl)octyl)oxy)-4-hydroxy-5-(2-hydroxyacetamido)tetrahydro-2H-pyran-2-carboxylic acid (259M) as white solid, Yield: 38 mg, 28%; LCMS (ESI+) m/z 1028.4 [M+H]⁺. ¹H NMR (400 MHz, MeOD) δ 8.00 (s, 1H), 7.47 (dd, J=22.0, 8.0 Hz, 4H), 6.83 (s, 2H), 6.76 (t, J=55.0 Hz, 4H), 4.66 (s, 2H), 4.42 (t, J=8.4 Hz, 2H), 4.04 (s, 2H), 3.92-3.62 (m, 20H), 3.51 (t, J=8.0 Hz, 2H), 3.47-3.44 (m, 2H), 3.37-3.32 (m, 1H), 2.77-2.71 (m, 1H), 2.48 (t, J=8.0 Hz, 2H), 1.92 (t, J=6.8 Hz, 2H), 1.75 (t, J=12 Hz, 1H), 1.55-1.54 (m, 2H), 1.34 (s, 9H)

### (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((6-(1-(2-oxo-6,9,12,15-tetraoxa-3-azaheptadecan-17-yl)-1H-1,2,3-triazol-4-yl)hexyl)oxy)tetrahydro-2H-pyran-2-carboxylic acid (Cpd. 253)

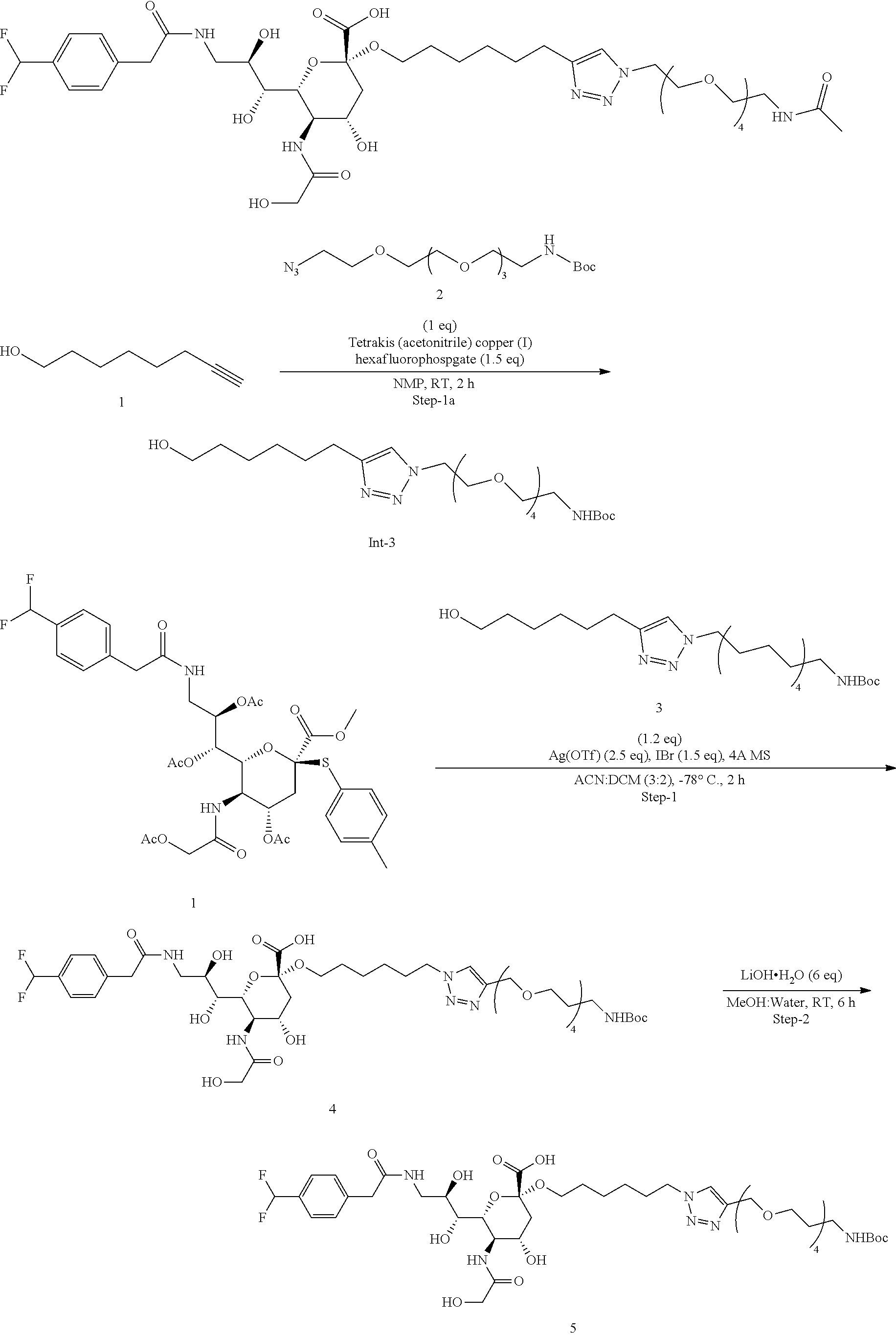

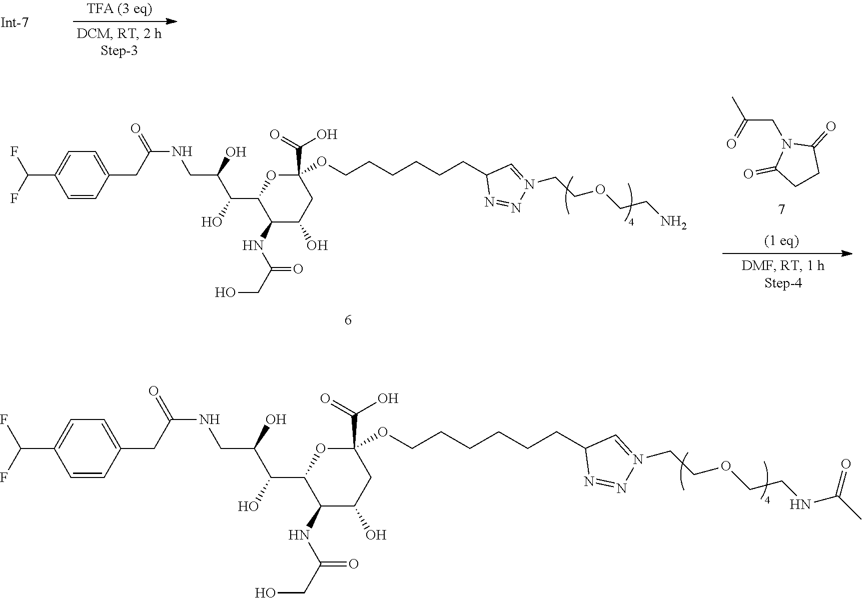

### Synthesis of tert-butyl (14-(4-(6-hydroxyhexyl)-1H-1,2,3-triazol-1-yl)-3,6,9,12-tetraoxatetradecyl) carbamate (3)

To a stirred solution of oct-7-yn-1-ol (1) (2 g, 15.84 mmol) and tert-butyl (14-azido-3,6,9,12-tetraoxatetradecyl)carbamate (2) (5.74 g, 15.84 mmol.) in NMP (20 mL) was added Tetrakis(acetonitrile)copper(I)hexafluorophosphate (7.4 g, 23.7 mmol, 1.5 eq.) and the reaction mixture was stirred at room temperature for 2 h. After completion, the reaction mixture was concentrated under reduced pressure and the crude residue obtained was purified by C18 reverse phase purification using acetonitrile and water (+0.05% TFA) as eluents to afford the desired compound tert-butyl (14-(4-(6-hydroxyhexyl)-1H-1,2,3-triazol-1-yl)-3,6,9,12-tetraoxatetradecyl)carbamate (3) as colorless semi solid, Yield: 4.1 g, 52%. ¹H NMR (400 MHz, DMSO) δ 7.80 (s, 1H), 6.73 (bs, 1H), 4.46 (t, J=5.6 Hz, 2H), 3.79 (t, J=5.6 Hz, 2H), 3.68-3.49 (m, 14H), 3.44-3.35 (m, 6H), 3.082-3.038 (m, 2H), 2.59 (t, J=7.6, Hz, 2H), 1.60-1.53 (m, 2H), 1.42-1.41 (m, 2H), 1.37 (s, 9H), 1.34-1.29 (m, 4H).

### Synthesis of (1R,2R)-1-((2R,3R,4S,6R)-4-acetoxy-3-(2-acetoxyacetamido)-6-((6-(1-(2,2-dimethyl-4-oxo-3,8,11,14,17-pentaoxa-5-azanonadecan-19-yl)-1H-1,2,3-triazol-4-yl)hexyl)oxy)-6-(methoxycarbonyl)tetrahydro-2H-pyran-2-yl)-3-(2-(4-(difluoromethyl) phenyl)acetamido) propane-1,2-diyl diacetate (4)

To a stirred solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-(difluoromethyl)phenyl)acetamido)propane-1,2-diyl diacetate (1) (2 g, 2.56 mmol) and tert-butyl (14-(4-(6-hydroxyhexyl)-1H-1,2,3-triazol-1-yl)-3,6,9,12-tetraoxatetradecyl)carbamate (3) (1.50 g, 3.07 mmol), Silver (1+) trifluoro methane sulfonate (AgOTf) (1.64 g, 6.4 mmol) in anhydrous dichloromethane (12 mL) and anhydrous acetonitrile (24 mL) was added activated 4 Å powdered molecular sieves (5 g) and the reaction mixture was stirred at room temperature for 1 h under nitrogen. The reaction mixture was cooled to −78° C., iodine monobromide (794 mg, 3.84 mmol) dissolved in dichloromethane (4 mL) was added dropwise and the resulting mixture was stirred at the same temperature for 2 h. After completion, the reaction mixture was quenched by addition of triethylamine (5 mL). The reaction mixture was filtered, the filtrate was washed with aqueous sodium thiosulfate solution, dried over sodium sulfate, filtered and concentrated under reduced pressure to get the crude residue which was purified by C18 reverse phase column chromatography using water and acetonitrile (+0.05% TFA) as eluents to afford the desired product (1R,2R)-1-((2R,3R,4S,6R)-4-acetoxy-3-(2-acetoxyacetamido)-6-((6-(1-(2,2-dimethyl-4-oxo-3,8,11,14,17-pentaoxa-5-azanonadecan-19-yl)-1H-1,2,3-triazol-4-yl)hexyl)oxy)-6-(methoxycarbonyl) tetrahydro-2H-pyran-2-yl)-3-(2-(4 (difluoromethyl)phenyl) acetamido)propane-1,2-diyl diacetate (4) as pale yellow solid, Yield: 1.2 g, 41%; LCMS (ESI+) m/z 1146.1 [M+H]⁺.

### Synthesis of (2R,4S,5R,6R)-6-((1R,2R)-3-(2-(4-(difluoromethyl)phenyl)acetamido)-1,2-dihydroxypropyl)-2-((6-(1-(2,2-dimethyl-4-oxo-3,8,11,14,17-pentaoxa-5-azanonadecan-19-yl)-1H-1,2,3-triazol-4-yl)hexyl)oxy)-4-hydroxy-5-(2-hydroxyacetamido)tetrahydro-2H-pyran-2-carboxylic acid (5)

To a stirred solution of (1R,2R)-1-((2R,3R,4S,6R)-4-acetoxy-3-(2-acetoxyacetamido)-6-((6-(1-(2,2-dimethyl-4-oxo-3,8,11,14,17-pentaoxa-5-azanonadecan-19-yl)-1H-1,2,3-triazol-4-yl)hexyl)oxy)-6-(methoxycarbonyl)tetrahydro-2H-pyran-2-yl)-3-(2-(4-(difluoromethyl)phenyl)acetamido)propane-1,2-diyl diacetate (4) (1.2 g, 1.02 mmol) in methanol (10 mL) and water (3 mL) was added LiOH·H₂O (302 mg, 6.12 mmol) at 0° C. The resulting reaction mixture was stirred at room temperature for 6 h. After completion, the reaction mixture was treated with acidic resin (Dowex 50, H⁺) to maintain pH˜6 and the suspension was filtered. The filtrate was concentrated under reduced pressure and the crude was purified by C18 reverse phase column chromatography using water and acetonitrile (+0.05% TFA) as eluents to afford the desired product (2R,4S,5R,6R)-6-((1R,2R)-3-(2-(4-(difluoromethyl)phenyl)acetamido)-1,2-dihydroxypropyl)-2-((6-(1-(2,2-dimethyl-4-oxo-3,8,11,14,17-pentaoxa-5-azanonadecan-19-yl)-1H-1,2,3-triazol-4-yl)hexyl)oxy)-4-hydroxy-5-(2-hydroxyacetamido)tetrahydro-2H-pyran-2-carboxylic acid (5) as an yellow solid, Yield: 720 mg, 76%; LCMS (ESI+) m/z 964.0 [M+H]⁺.

### Synthesis of (2R,4S,5R,6R)-2-((6-(1-(14-amino-3,6,9,12-tetraoxatetradecyl)-1H-1,2,3-triazol-4-yl)hexyl)oxy)-6-((1R,2R)-3-(2-(4-(difluoromethyl)phenyl)acetamido)-1,2-dihydroxypropyl)-4-hydroxy-5-(2-hydroxyacetamido)tetrahydro-2H-pyran-2-carboxylic acid (6)

To a stirred solution of (2R,4S,5R,6R)-6-((1R,2R)-3-(2-(4-(difluoromethyl)phenyl)acetamido)-1,2-dihydroxypropyl)-2-((6-(1-(2,2-dimethyl-4-oxo-3,8,11,14,17-pentaoxa-5-azanonadecan-19-yl)-1H-1,2,3-triazol-4-yl)hexyl)oxy)-4-hydroxy-5-(2-hydroxyacetamido)tetrahydro-2H-pyran-2-carboxylic acid (5) (720 mg, 0.74 mmol) in anhydrous dichloromethane (7.2 mL) was added trifluoro acetic acid (2 mL) dropwise at 0° C. and the resulting reaction mixture was stirred at room temperature for 2 h. After completion, the solvent was concentrated and the crude residue obtained was purified by C18 reverse phase column chromatography using water and acetonitrile (+0.05% TFA) as eluents to afford the desired product (2R,4S,5R,6R)-2-((6-(1-(14-amino-3,6,9,12-tetraoxatetradecyl)-1H-1,2,3-triazol-4-yl)hexyl)oxy)-6-((1R,2R)-3-(2-(4-(difluoromethyl) phenyl)acetamido)-1,2-dihydroxypropyl)-4-hydroxy-5-(2-hydroxyacetamido)tetrahydro-2H-pyran-2-carboxylic acid (6) as a yellow solid, Yield: 320 mg, 49%; LCMS (ESI+) m/z 863.9 [M+H]⁺.

### Synthesis of (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((6-(1-(2-oxo-6,9,12,15-tetraoxa-3-azaheptadecan-17-yl)-1H-1,2,3-triazol-4-yl)hexyl)oxy)tetrahydro-2H-pyran-2-carboxylic acid (253)

To a stirred solution of (2R,4S,5R,6R)-2-((6-(1-(14-amino-3,6,9,12-tetraoxatetradecyl)-1H-1,2,3-triazol-4-yl)hexyl)oxy)-6-((1R,2R)-3-(2-(4-(difluoromethyl)phenyl)acetamido)-1,2-dihydroxypropyl)-4-hydroxy-5-(2-hydroxyacetamido)tetrahydro-2H-pyran-2-carboxylic acid (6) (70 mg, 0.08 mmol) in DMF (0.7 mL) was added 2,5-dioxopyrrolidin-1-yl acetate (7) (12.7 mg, 0.08 mmol) at 0° C. The resulting reaction mixture was stirred at 0° C. for 1 h. After completion, the reaction mixture was purified by prep-HPLC using water and acetonitrile (+0.05% TFA) to afford the desired product (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((6-(1-(2-oxo-6,9,12,15-tetraoxa-3-azaheptadecan-17-yl)-1H-1,2,3-triazol-4-yl)hexyl)oxy)tetrahydro-2H-pyran-2-carboxylic acid (253) as a white solid, Yield: 15 mg, 20.4%; LCMS (ESI+) m/z 905.9 [M+H]⁺. ¹H NMR (400 MHz, MeOD) δ 7.88 (s, 1H), 7.46 (dd, J=22.0, 8.0 Hz, 4H), 6.76 (t, J=56 Hz, 1H), 4.58 (t, J=5.2 Hz, 2H), 4.04 (s, 2H), 3.92-3.89 (m, 4H), 3.84-3.79 (m, 2H), 3.74-3.73 (m, 1H), 3.71-3.67 (m, 2H), 3.67-3.67 (m, 1H), 3.66-3.65 (m, 3H), 3.66-3.61 (m, 8H), 3.55 (t, J=5.2 Hz, 2H), 3.44-3.40 (m, 2H), 3.39-3.36 (m, 2H), 2.72 (dd, J=7.6, 3.0 Hz, 1H), 2.75-2.71 (m, 4H), 1.96 (s, 1H), 1.72-1.69 (m, 1H), 1.73-1.65 (m, 2H), 1.62-1.52 (m, 2H), 1.49-1.36 (m, 4H)

### (2R,4S,5R,6R)-6-((1R,2R)-3-(2-(4-(difluoromethyl)phenyl)acetamido)-1,2-dihydroxypropyl)-2-((6-(1-(18-(2,5-dioxo-2,5-dihydro-1H-pyrrol-1-yl)-16-oxo-3,6,9,12-tetraoxa-15-azaoctadecyl)-1H-1,2,3-triazol-4-yl)hexyl)oxy)-4-hydroxy-5-(2-hydroxyacetamido)tetrahydro-2H-pyran-2-carboxylic acid (Cpd. 253M)

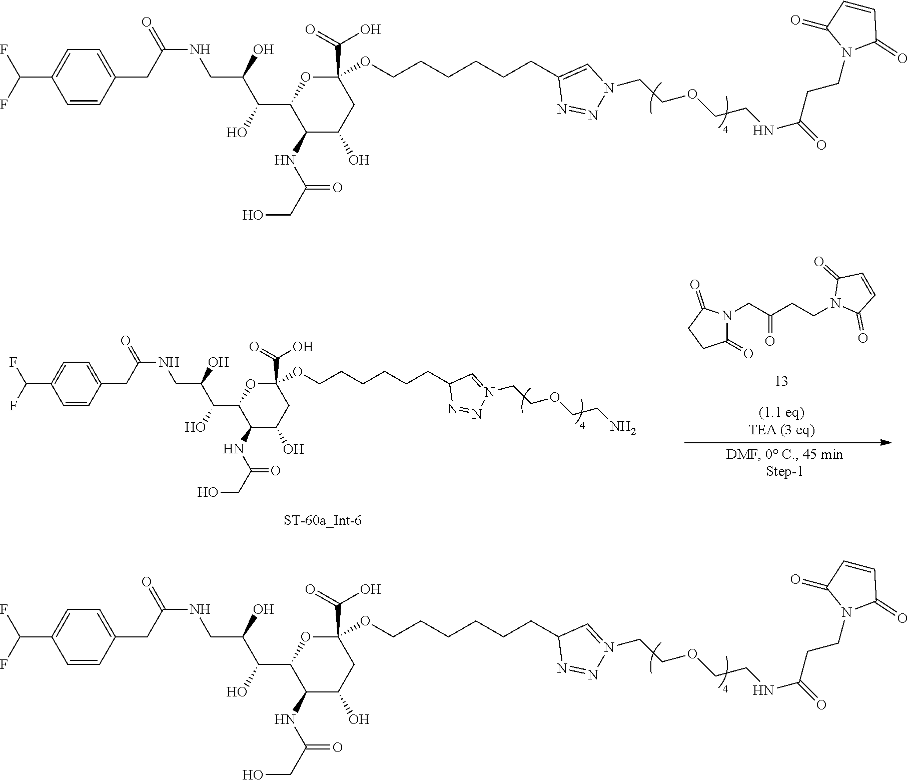

### Synthesis of (2R,4S,5R,6R)-6-((1R,2R)-3-(2-(4-(difluoromethyl)phenyl)acetamido)-1,2-dihydroxypropyl)-2-((6-(1-(18-(2,5-dioxo-2,5-dihydro-1H-pyrrol-1-yl)-16-oxo-3,6,9,12-tetraoxa-15-azaoctadecyl)-1H-1,2,3-triazol-4-yl)hexyl)oxy)-4-hydroxy-5-(2-hydroxyacetamido)tetrahydro-2H-pyran-2-carboxylic acid (253M)

To a stirred solution of (2R,4S,5R,6R)-2-((6-(1-(14-amino-3,6,9,12-tetraoxatetradecyl)-1H-1,2,3-triazol-4-yl)hexyl)oxy)-6-((1R,2R)-3-(2-(4-(difluoromethyl)phenyl)acetamido)-1,2-dihydroxypropyl)-4-hydroxy-5-(2-hydroxyacetamido)tetrahydro-2H-pyran-2-carboxylic acid (253_Int-6) (150 mg, 0.17 mmol) in DMF (1.5 mL) were added 2,5-dioxopyrrolidin-1-yl 3-(2,5-dioxo-2,5-dihydro-1H-pyrrol-1-yl)propanoate (1) (50 mg, 0.19 mmol) and triethylamine (0.30 mL, 0.52 mmol) at 0° C. The resulting reaction mixture was stirred at the same temperature for 1 h. After completion, the reaction mixture was purified by prep-HPLC using water and acetonitrile (+0.05% TFA) as eluents to afford the desired product (2R,4S,5R,6R)-6-((1R,2R)-3-(2-(4-(difluoromethyl)phenyl)acetamido)-1,2-dihydroxypropyl)-2-((6-(1-(18-(2,5-dioxo-2,5-dihydro-1H-pyrrol-1-yl)-16-oxo-3,6,9,12-tetraoxa-15-azaoctadecyl)-1H-1,2,3-triazol-4-yl)hexyl)oxy)-4-hydroxy-5-(2-hydroxyacetamido)tetrahydro-2H-pyran-2-carboxylic acid (253M) as a white solid, Yield: 40 mg, 22%; LCMS (ESI+) m/z 1015.00 [M+H]⁺. ¹H NMR (400 MHz, MeOD) δ 7.82 (s, 1H), 7.46 (dd, J=22.0, 8.0 Hz, 4H), 6.82 (s, 1H), 6.74 (t, J=56 Hz, 1H), 4.56 (t, J=5.2 Hz, 2H), 4.03 (s, 2H), 3.91-3.88 (m, 3H), 3.83-3.81 (m, 1H), 3.81-3.73 (m, 5H), 3.69-3.62 (m, 15H), 3.50 (t, J=5.2 Hz, 2H), 3.49-3.46 (m, 2H), 3.39-3.27 (m, 2H), 2.73-2.69 (m, 3H), 2.47 (t, J=7.2 Hz, 2H), 1.74-1.68 (m, 3H), 1.61-1.51 (m, 2H), 1.49-1.32 (m, 5H).

### (2R,4S,5R,6R)-6-((1R,2R)-3-(2-(4-(difluoromethyl)phenyl)acetamido)-1,2-dihydroxypropyl)-2-((6-(1-(16,32-dioxo-36-((3aR,4R,6aS)-2-oxohexahydro-1H-thieno[3,4-d]imidazol-4-yl)-3,6,9,12,19,22,25,28-octaoxa-15,31-diazahexatriacontyl)-1H-1,2,3-triazol-4-yl)hexyl)oxy)-4-hydroxy-5-(2-hydroxyacetamido)tetrahydro-2H-pyran-2-carboxylic acid (Cpd. 253B)

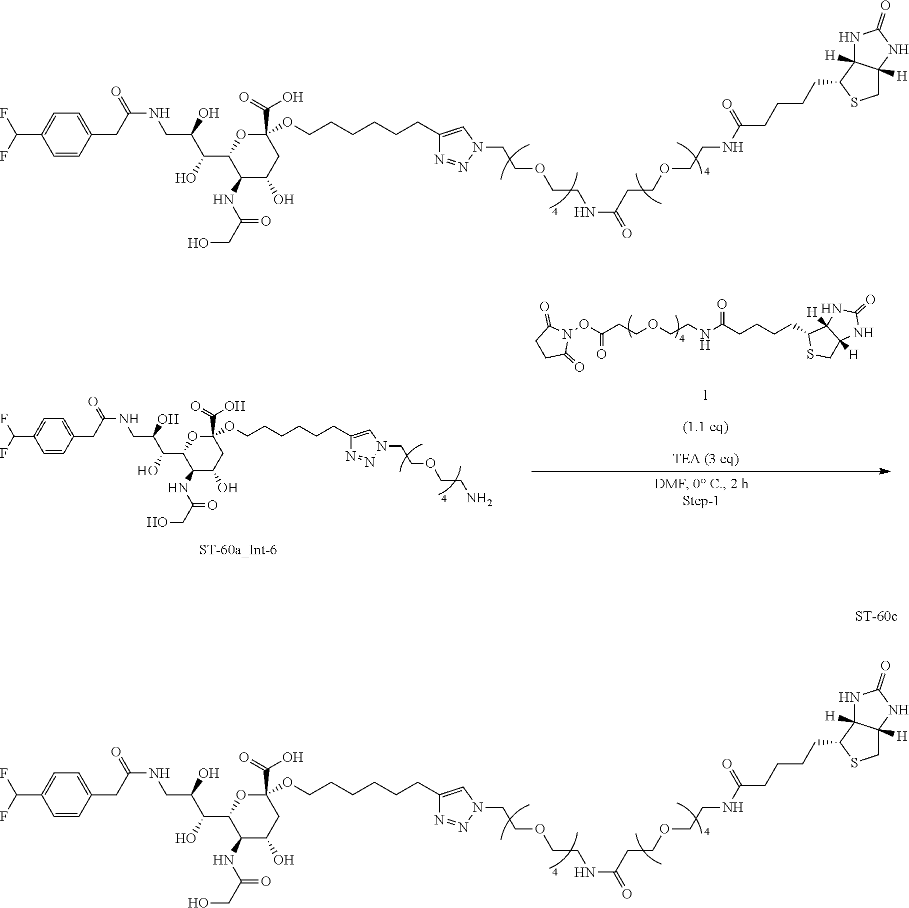

### Synthesis of (2R,4S,5R,6R)-6-((1R,2R)-3-(2-(4-(difluoromethyl)phenyl)acetamido)-1,2-dihydroxypropyl)-2-((6-(1-(16,32-dioxo-36-((3aR,4R,6aS)-2-oxohexahydro-1H-thieno[3,4-d]imidazol-4-yl)-3,6,9,12,19,22,25,28-octaoxa-15,31-diazahexatriacontyl)-1H-1,2,3-triazol-4-yl)hexyl)oxy)-4-hydroxy-5-(2-hydroxyacetamido)tetrahydro-2H-pyran-2-carboxylic acid (253B)

To a stirred solution of (2R,4S,5R,6R)-6-((1R,2R)-3-(2-(4-(1H-tetrazol-5-yl)phenyl)acetamido)-1,2-dihydroxypropyl)-4-hydroxy-5-(2-hydroxyacetamido)-2-((1,1,1-trifluoro-2,19-dioxo-6,9,12,15-tetraoxa-314,18-diazapentacosan-25-yl)oxy)tetrahydro-2H-pyran-2-carboxylic acid (253_Int-6) (50 mg, 0.05 mmol) in anhydrous N,N-dimethyl formamide (0.5 mL) were added 2,5-dioxopyrrolidin-1-yl 17-oxo-21-((3aR,4R,6aS)-2-oxohexahydro-1H-thieno[3,4-d]imidazol-4-yl)-4,7,10,13-tetraoxa-16-azahenicosanoate (1) (37 mg, 0.06 mmol) and triethylamine (0.02 ml, 0.15 mmol) at 0° C. The resulting reaction mixture was stirred at the same temperature for 2 h. After completion, the reaction mixture was purified by prep-HPLC by using acetonitrile and water (+0.05%, TFA) as eluents to afford the desired product (2R,4S,5R,6R)-6-((1R,2R)-3-(2-(4-(difluoromethyl)phenyl)acetamido)-1,2-dihydroxypropyl)-2-((6-(1-(16,32-dioxo-36-((3aR,4R,6aS)-2-oxohexahydro-1H-thieno[3,4-d]imidazol-4-yl)-3,6,9,12,19,22,25,28-octaoxa-15,31-diazahexatriacontyl)-1H-1,2,3-triazol-4-yl)hexyl)oxy)-4-hydroxy-5-(2-hydroxyacetamido)tetrahydro-2H-pyran-2-carboxylic acid (253B). as white solid, Yield: 60 mg, 77%. LCMS (ESI−) m/z 1335.1 [M−H]−. ¹H NMR (400 MHz, MeOD) δ 7.87 (s, 1H), 7.46 (dd, J=22.0, 8.0 Hz, 4H), 6.75 (t, J=56 Hz, 1H), 4.58 (t, J=5.2 Hz, 2H), 4.52-4.49 (m, 1H), 4.33-4.29 (m, 1H), 4.03 (s, 2H), 3.92-3.89 (m, 3H), 3.83-3.72 (m, 3H), 3.74-3.71 (m, 4H), 3.65-3.62 (m, 26H), 3.55-3.54 (m, 4H), 3.44-3.32 (m, 6H), 3.41-3.31 (m, 2H), 3.99-3.91 (m, 1H), 2.74-2.70 (m, 4H), 2.47 (t, J=6.4 Hz, 1H), 2.23 (t, J=7.2 Hz, 1H), 1.74-1.65 (m, 6H), 1.61-1.51 (m, 3H), 1.51-1.32 (m, 6H).

### (2R,4S,5R,6R)-6-((1R,2R)-3-(2-(4-chlorophenyl)acetamido)-1,2-dihydroxypropyl)-4-hydroxy-5-(2-hydroxyacetamido)-2-(2-(2-(2-(4-(15-oxo-2,5,8,11-tetraoxa-14-azahexadecyl)-1H-1,2,3-triazol-1-yl)ethoxy)ethoxy)ethoxy)tetrahydro-2H-pyran-2-carboxylic acid (Cpd. ST-61a)

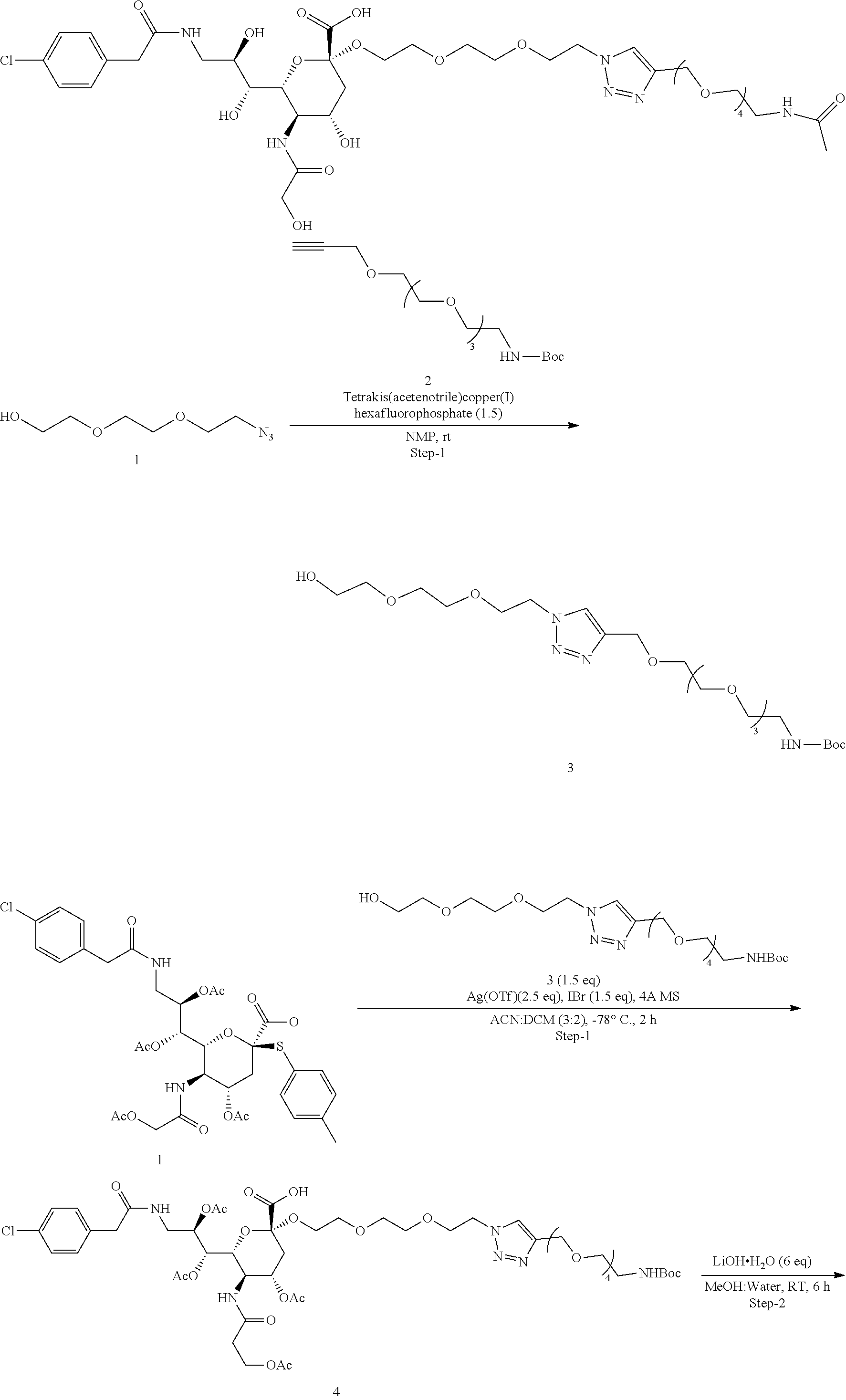

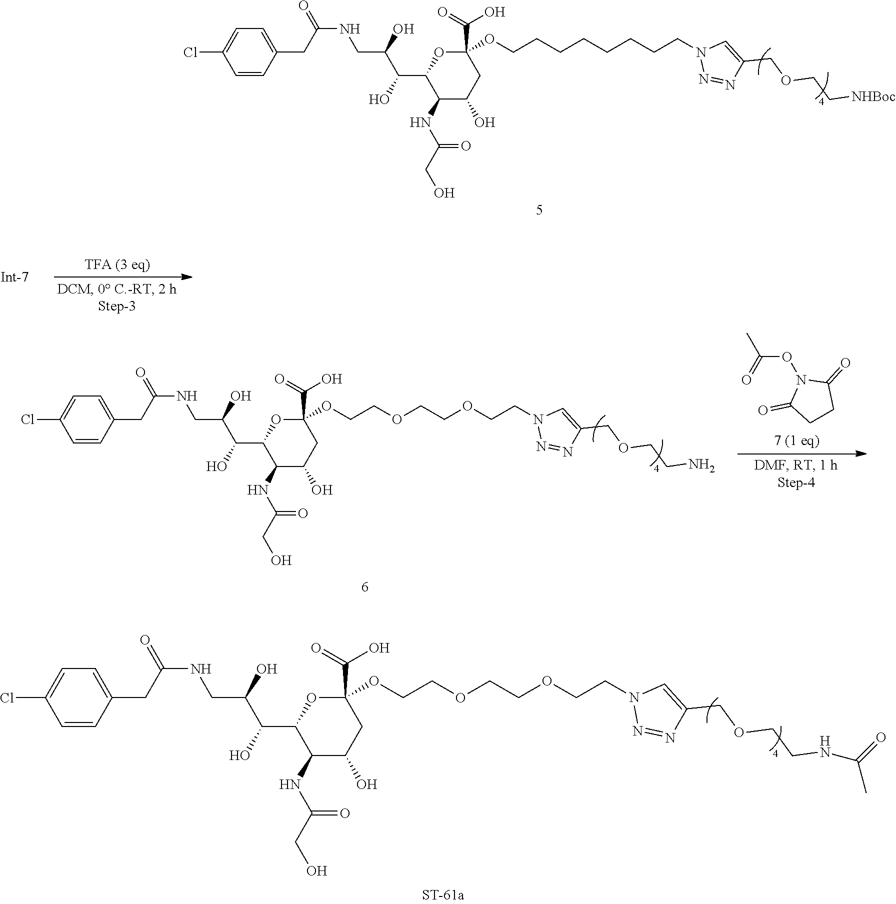

### Synthesis of tert-butyl (1-(1-(2-(2-(2-hydroxyethoxy)ethoxy)ethyl)-1H-1,2,3-triazol-4-yl)-2,5,8,11-tetraoxatridecan-13-yl) carbamate (3)

To a stirred solution of 2-(2-(2-azidoethoxy) ethoxy) ethan-1-ol (1) (1 g, 5.7 mmol) and tert-butyl (3,6,9,12-tetraoxapentadec-14-yn-1-yl) carbamate (2) (2.31 g, 5.7 mmol) in anhydrous N-methyl pyrrolidone (10 mL) was added Tetrakis(acetonitrile)copper(1) hexa-fluoro phosphate (3.19 g, 5.8 mmol) under nitrogen. The reaction mixture was stirred at room temperature for 2 h. After completion, the mixture was filtered, few drops of AcOH were added and the filtrate was purified by reverse phase column chromatography using water and acetonitrile (+0.05% TFA) as eluents to afford tert-butyl (1-(1-(2-(2-(2-hydroxyethoxy)ethoxy)ethyl)-1H-1,2,3-triazol-4-yl)-2,5,8,11-tetraoxatridecan-13-yl)carbamate (ST-29a_Int-3) (Yield: 1.8 g, 62%). ¹H NMR (400 MHz DMSO-d6): δ 8.07 (s, 1H), 6.75 (s, 1H), 4.52-4.50 (m, 4H), 3.81 (t, J=5.2 Hz, 2H), 3.55-3.53 (m, 10H), 3.54-3.43 (m, 7H), 3.93-3.35 (m, 6H), 3.08-3.03 (m, 2H), 1.37 (s, 9H).

### Synthesis of (1R,2R)-1-((2R,3R,4S,6R)-4-acetoxy-3-(2-acetoxyacetamido)-6-(2-(2-(2-(4-(17,17-dimethyl-15-oxo-2,5,8,11,16-pentaoxa-14-aza° C. tadecyl)-1H-1,2,3-triazol-1-yl)ethoxy)ethoxy)ethoxy)-6-(methoxycarbonyl)tetrahydro-2H-pyran-2-yl)-3-(2-(4-chlorophenyl)acetamido)propane-1,2-diyl diacetate (4)

To a stirred solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (1) (2 g, 2.61 mmol), tert-butyl (1-(1-(2-(2-(2-hydroxyethoxy)ethoxy)ethyl)-1H-1,2,3-triazol-4-yl)-2,5,8,11-tetraoxatridecan-13-yl)carbamate (3) (1.99 g, 3.92 mmol), Silver (1+) trifluoro methane sulfonate (AgOTf) (1.67 g, 6.52 mmol) in anhydrous dichloromethane (12 mL) and anhydrous acetonitrile (24 mL) was added activated 4 Å powdered molecular sieves (5 g). The reaction mixture was stirred at room temperature for 1 h under nitrogen. The solution was cooled to −78° C., iodine monobromide (806 mg, 3.91 mmol) dissolved in dichloromethane (4 mL) was added dropwise and the resulting mixture was stirred at same temperature for 2 h. After completion, the reaction mixture was quenched by addition of triethylamine (5 mL) at the same temperature and warmed to room temperature. The reaction mixture was filtered, the filtrate was washed with 10% aqueous sodium thiosulfate solution, dried over sodium sulfate, filtered, concentrated under reduced pressure. The crude residue obtained was purified by C18 reverse phase column chromatography using water and acetonitrile (+0.05% TFA) as eluents to afford the desired product (1R,2R)-1-((2R,3R,4S,6R)-4-acetoxy-3-(2-acetoxyacetamido)-6-(2-(2-(2-(4-(17,17-dimethyl-15-oxo-2,5,8,11,16-pentaoxa-14-aza° C. tadecyl)-1H-1,2,3-triazol-1-yl)ethoxy)ethoxy)ethoxy)-6-(methoxycarbonyl)tetrahydro-2H-pyran-2-yl)-3-(2-(4-chlorophenyl)acetamido)propane-1,2-diyl diacetate (4) as a pale yellow solid, Yield- 900 mg, 30.1%. LCMS (ESI) m/z 1148.0 [M+H]⁺.

### Synthesis of (2R,4S,5R,6R)-6-((1R,2R)-3-(2-(4-chlorophenyl)acetamido)-1,2-dihydroxypropyl)-2-(2-(2-(2-(4-(17,17-dimethyl-15-oxo-2,5,8,11,16-pentaoxa-14-aza° C. tadecyl)-1H-1,2,3-triazol-1-yl)ethoxy)ethoxy)ethoxy)-4-hydroxy-5-(2-hydroxyacetamido)tetrahydro-2H-pyran-2-carboxylic acid (5)

To a stirred solution of (1R,2R)-1-((2R,3R,4S,6R)-4-acetoxy-3-(2-acetoxyacetamido)-6-(2-(2-(2-(4-(17,17-dimethyl-15-oxo-2,5,8,11,16-pentaoxa-14-aza° C. tadecyl)-1H-1,2,3-triazol-1-yl)ethoxy)ethoxy)ethoxy)-6-(methoxycarbonyl)tetrahydro-2H-pyran-2-yl)-3-(2-(4-chlorophenyl)acetamido)propane-1,2-diyl diacetate (4) (900 mg, 0.78 mmol) in methanol (9 mL) was added lithium hydroxide monohydrate (196 mg, 4.68 mmol, 6 eq.) in water (1 mL) at 0° C. and the reaction mixture was stirred at room temperature for 16 h. After completion, the reaction mixture was treated with acidic resin (Dowex 50, H⁺) to maintain pH˜6 and the suspension was filtered. The filtrate was concentrated under reduced pressure and the crude residue obtained was purified by C18 reverse phase column chromatography using water and acetonitrile (+0.05% TFA) as eluents to afford the desired product (2R,4S,5R,6R)-6-((1R,2R)-3-(2-(4-chlorophenyl)acetamido)-1,2-dihydroxypropyl)-2-(2-(2-(2-(4-(17,17-dimethyl-15-oxo-2,5,8,11,16-pentaoxa-14-aza° C. tadecyl)-1H-1,2,3-triazol-1-yl)ethoxy)ethoxy)ethoxy)-4-hydroxy-5-(2-hydroxyacetamido)tetrahydro-2H-pyran-2-carboxylic acid (5) as an yellow gummy solid, Yield: 530 mg, 70%. LCMS (ESI) m/z 965.9 [M+H]⁺.

### Synthesis of (2R,4S,5R,6R)-2-(2-(2-(2-(4-(13-amino-2,5,8,11-tetraoxatridecyl)-1H-1,2,3-triazol-1-yl)ethoxy)ethoxy)ethoxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (6)

To a stirred solution of (2R,4S,5R,6R)-6-((1R,2R)-3-(2-(4-chlorophenyl)acetamido)-1,2-dihydroxypropyl)-2-(2-(2-(2-(4-(17,17-dimethyl-15-oxo-2,5,8,11,16-pentaoxa-14-aza° C. tadecyl)-1H-1,2,3-triazol-1-yl)ethoxy)ethoxy)ethoxy)-4-hydroxy-5-(2-hydroxyacetamido)tetrahydro-2H-pyran-2-carboxylic acid (5) (530 mg, 0.54 mmol) in anhydrous dichloromethane (8 mL) was added trifluoro acetic acid (1.1 mL) dropwise at 0° C. and the resulting reaction mixture was stirred at room temperature for 3 h. After completion, the solvent was concentrated and the crude residue obtained was purified by prep-HPLC using acetonitrile and water (+0.05% TFA) as eluents to afford the desired product (2R,4S,5R,6R)-2-(2-(2-(2-(4-(13-amino-2,5,8,11-tetraoxatridecyl)-1H-1,2,3-triazol-1-yl)ethoxy)ethoxy)ethoxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (6) as a yellow solid, Yield: 280 mg, 59%. LCMS (ESI+) m/z 863.8 [M+H]⁺.

### Synthesis of (2R,4S,5R,6R)-6-((1R,2R)-3-(2-(4-chlorophenyl)acetamido)-1,2-dihydroxypropyl)-4-hydroxy-5-(2-hydroxyacetamido)-2-(2-(2-(2-(4-(15-oxo-2,5,8,11-tetraoxa-14-azahexadecyl)-1H-1,2,3-triazol--yl)ethoxy)ethoxy)ethoxy)tetrahydro-2H-pyran-2-carboxylic acid (ST-61a)

To a stirred solution of (2R,4S,5R,6R)-2-(2-(2-(2-(4-(13-amino-2,5,8,11-tetraoxatridecyl)-1H-1,2,3-triazol-1-yl)ethoxy)ethoxy)ethoxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (6) (70 mg, 0.080 mmol) in DMF (0.7 mL) was added 2,5-dioxopyrrolidin-1-yl acetate (7) (12.71 mg, 0.080 mmol) at 0° C. The resulting reaction mixture was stirred at the same temperature for 1 h. After completion, the reaction mixture was purified by prep-HPLC using acetonitrile and water (+0.05% TFA) to afford the desired product (2R,4S,5R,6R)-6-((1R,2R)-3-(2-(4-chlorophenyl)acetamido)-1,2-dihydroxypropyl)-4-hydroxy-5-(2-hydroxyacetamido)-2-(2-(2-(2-(4-(15-oxo-2,5,8,11-tetraoxa-14-azahexadecyl)-1H-1,2,3-triazol-1-yl)ethoxy)ethoxy)ethoxy)tetrahydro-2H-pyran-2-carboxylic acid (ST-61a) as white solid, Yield: 85 mg, 41%. LCMS (ESI+) m/z 905.8 [M+H]⁺. ¹H NMR (400 MHz, MeOD) δ 8.05 (s, 1H), 7.33-7.28 (m, 4H), 4.65 (s, 2H), 4.60 (t, J=4.8 Hz, 2H), 4.03 (s, 2H), 3.92-3.89 (m, 4H), 3.87-3.82 (m, 2H), 3.75-3.73 (m, 2H), 3.73-3.70 (m, 4H), 3.70-3.67 (m, 7H), 3.66-3.61 (m, 8H), 3.59-3.52 (m, 4H), 3.40-3.33 (m, 3H), 3.32-3.25 (m, 2H), 2.76-2.70 (m, 1H), 1.95 (s, 1H), 1.76 (t, J=12.4 Hz, 1H).

### (2R,4S,5R,6R)-6-((1R,2R)-3-(2-(4-chlorophenyl)acetamido)-1,2-dihydroxypropyl)-2-(2-(2-(2-(4-(17-(2,5-dioxo-2,5-dihydro-1H-pyrrol-1-yl)-15-oxo-2,5,8,11-tetraoxa-14-azaheptadecyl)-1H-1,2,3-triazol-1-yl)ethoxy)ethoxy)ethoxy)-4-hydroxy-5-(2-hydroxyacetamido)tetrahydro-2H-pyran-2-carboxylic acid (Cpd. 260M)

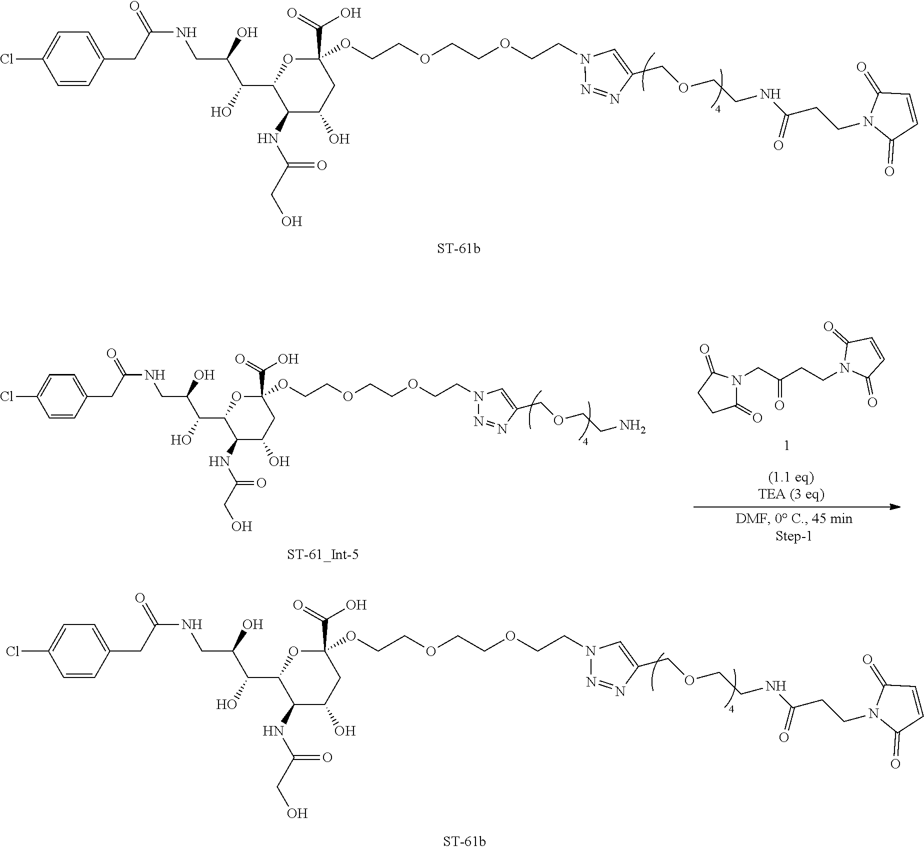

### Synthesis of (2R,4S,5R,6R)-6-((1R,2R)-3-(2-(4-chlorophenyl)acetamido)-1,2-dihydroxypropyl)-2-(2-(2-(2-(4-(17-(2,5-dioxo-2,5-dihydro-1H-pyrrol-1-yl)-15-oxo-2,5,8,11-tetraoxa-14-azaheptadecyl)-1H-1,2,3-triazol-1-yl)ethoxy)ethoxy)ethoxy)-4-hydroxy-5-(2-hydroxyacetamido)tetrahydro-2H-pyran-2-carboxylic acid (260M)

To a stirred solution of (2R,4S,5R,6R)-2-(2-(2-(2-(4-(13-amino-2,5,8,11-tetraoxatridecyl)-1H-1,2,3-triazol-1-yl)ethoxy)ethoxy)ethoxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (ST-61a_Int-6) (150 mg, 0.17 mmol) in DMF (1.5 mL) were added 2,5-dioxopyrrolidin-1-yl 3-(2,5-dioxo-2,5-dihydro-1H-pyrrol-1-yl)propanoate (1) (41.8 mg, 0.19 mmol) and triethylamine (0.07 mL, 0.51 mmol) at 0° C. The resulting reaction mixture was stirred at the same temperature for 1 h. After completion, the reaction mixture was purified by prep-HPLC to afford the desired material (2R,4S,5R,6R)-6-((1R,2R)-3-(2-(4-chlorophenyl)acetamido)-1,2-dihydroxypropyl)-2-(2-(2-(2-(4-(17-(2,5-dioxo-2,5-dihydro-1H-pyrrol-1-yl)-15-oxo-2,5,8,11-tetraoxa-14-azaheptadecyl)-1H-1,2,3-triazol-1-yl)ethoxy)ethoxy)ethoxy)-4-hydroxy-5-(2-hydroxyacetamido)tetrahydro-2H-pyran-2-carboxylic acid (260M) as a white solid, Yield: 55 mg, 31%; LCMS (ESI+) m/z 1016.4 [M+H]⁺. ¹H NMR (400 MHz, MeOD) δ 8.05 (s, 1H), 7.31-7.30 (m, 4H), 6.82 (s, 2H), 4.65 (s, 2H), 4.60 (t, J=4.8 Hz, 2H), 4.03 (s, 2H), 3.92-3.88 (m, 4H), 3.87-3.83 (m, 2H), 3.76-3.70 (m, 4H), 3.68-3.61 (m, 11H), 3.63-3.59 (m, 8H), 3.54 (s, 2H), 3.50 (t, J=6.4 Hz, 2H), 3.40-3.31 (m, 1H), 3.29-3.21 (m, 1H), 2.47 (t, J=6.8 Hz, 2H), 1.77 (t, J=12.4 Hz, 1H).

### (2R,4S,5R,6R)-6-((1R,2R)-3-(2-(4-chlorophenyl)acetamido)-1,2-dihydroxypropyl)-2-(2-(2-(2-(4-(15,31-dioxo-35-((3aR,4R,6aS)-2-oxohexahydro-1H-thieno[3,4-d]imidazol-4-yl)-2,5,8,11,18,21,24,27-° C. taoxa-14,30-diazapentatriacontyl)-1H-1,2,3-triazol-1-yl)ethoxy)ethoxy)ethoxy)-4-hydroxy-5-(2-hydroxyacetamido)tetrahydro-2H-pyran-2-carboxylic acid (Cpd. 260B)

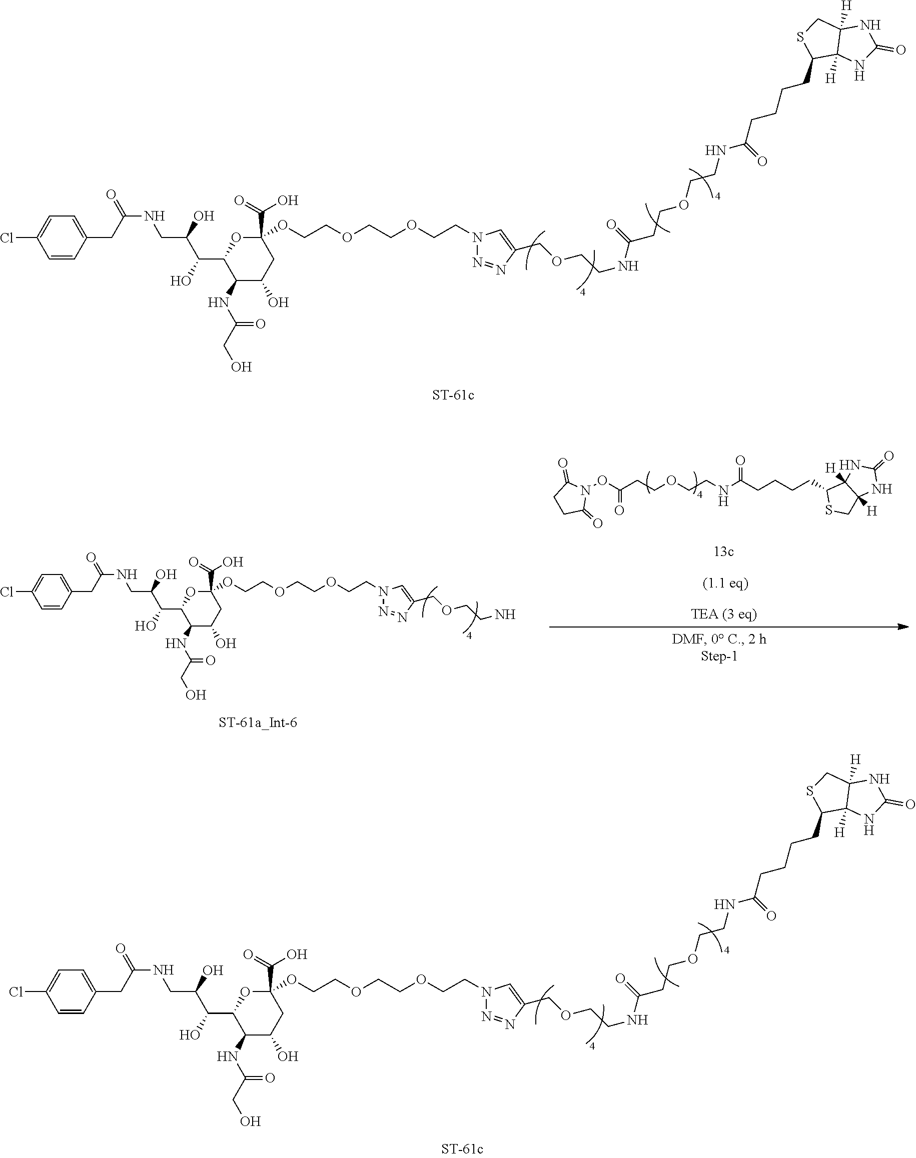

### Synthesis of (2R,4S,5R,6R)-6-((1R,2R)-3-(2-(4-chlorophenyl)acetamido)-1,2-dihydroxypropyl)-2-(2-(2-(2-(4-(15,31-dioxo-35-((3aR,4R,6aS)-2-oxohexahydro-1H-thieno[3,4-d]imidazol-4-yl)-2,5,8,11,18,21,24,27-° C. taoxa-14,30-diazapentatriacontyl)-1H-1,2,3-triazol-1-yl)ethoxy)ethoxy)ethoxy)-4-hydroxy-5-(2-hydroxyacetamido)tetrahydro-2H-pyran-2-carboxylic acid (260B)

To a stirred solution of (2R,4S,5R,6R)-2-(2-(2-(2-(4-(13-amino-2,5,8,11-tetraoxatridecyl)-1H-1,2,3-triazol-1-yl)ethoxy)ethoxy)ethoxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (ST-61a_Int-6) (50 mg, 0.05 mmol) in anhydrous N,N-dimethyl formamide (0.5 mL) were added 2,5-dioxopyrrolidin-1-yl 17-oxo-21-((3aR,4R,6aS)-2-oxohexahydro-1H-thieno[3,4-d]imidazol-4-yl)-4,7,10,13-tetraoxa-16-azahenicosanoate (1) (37 mg, 0.06 mmol) and triethylamine (0.02 ml, 0.15 mmol) at 0° C. The resulting reaction mixture was stirred at the same temperature for 2 h. After completion, the reaction mixture was purified by prep-HPLC using acetonitrile and water (+0.05%, TFA) as eluents to afford the desired product (2R,4S,5R,6R)-6-((1R,2R)-3-(2-(4-chlorophenyl)acetamido)-1,2-dihydroxypropyl)-2-(2-(2-(2-(4-(15,31-dioxo-35-((3aR,4R,6aS)-2-oxohexahydro-1H-thieno[3,4-d]imidazol-4-yl)-2,5,8,11,18,21,24,27-° C. taoxa-14,30-diazapentatriacontyl)-1H-1,2,3-triazol-1-yl)ethoxy)ethoxy)ethoxy)-4-hydroxy-5-(2-hydroxyacetamido)tetrahydro-2H-pyran-2-carboxylic acid (260B) as white solid, Yield: 45 mg, 48%. LCMS (ESI−) m/z 1339.4 [M+H]⁺. ¹H NMR (400 MHz, MeOD) δ 8.06 (s, 1H), 7.31-7.30 (m, 4H), 4.66 (s, 1H), 4.60 (t, J=4.8 Hz, 2H), 4.53-4.48 (m, 2H), 4.37-4.30 (m, 2H), 4.04 (s, 2H), 3.92-3.83 (m, 4H), 3.88-3.83 (m, 2H), 3.72 (t, J=8.4 Hz, 2H), 3.67-3.58 (m, 30H), 3.56-3.53 (m, 8H), 3.40-3.32 (m, 5H), 3.38-3.31 (m, 2H), 2.96-2.91 (m, 1H), 2.73-2.70 (m, 2H), 2.47 (t, J=6 Hz, 2H), 2.23 (t, J=7.2 Hz, 2H), 1.29-1.21 (m, 2H), 1.19-1.10 (m, 3H), 1.49-1.42 (m, 2H).

### (2R,4S,5R,6R)-6-((1R,2R)-3-(2-(4-(difluoro methyl) phenyl) acetamido)-1,2-dihydroxypropyl)-2-((2,18-dioxo-6,9,12,15-tetraoxa-3,19-diazapentacosan-25-yl) oxy)-4-hydroxy-5-(2-hydroxyacetamido) tetrahydro-2H-pyran-2-carboxylic acid (Cpd. 256)

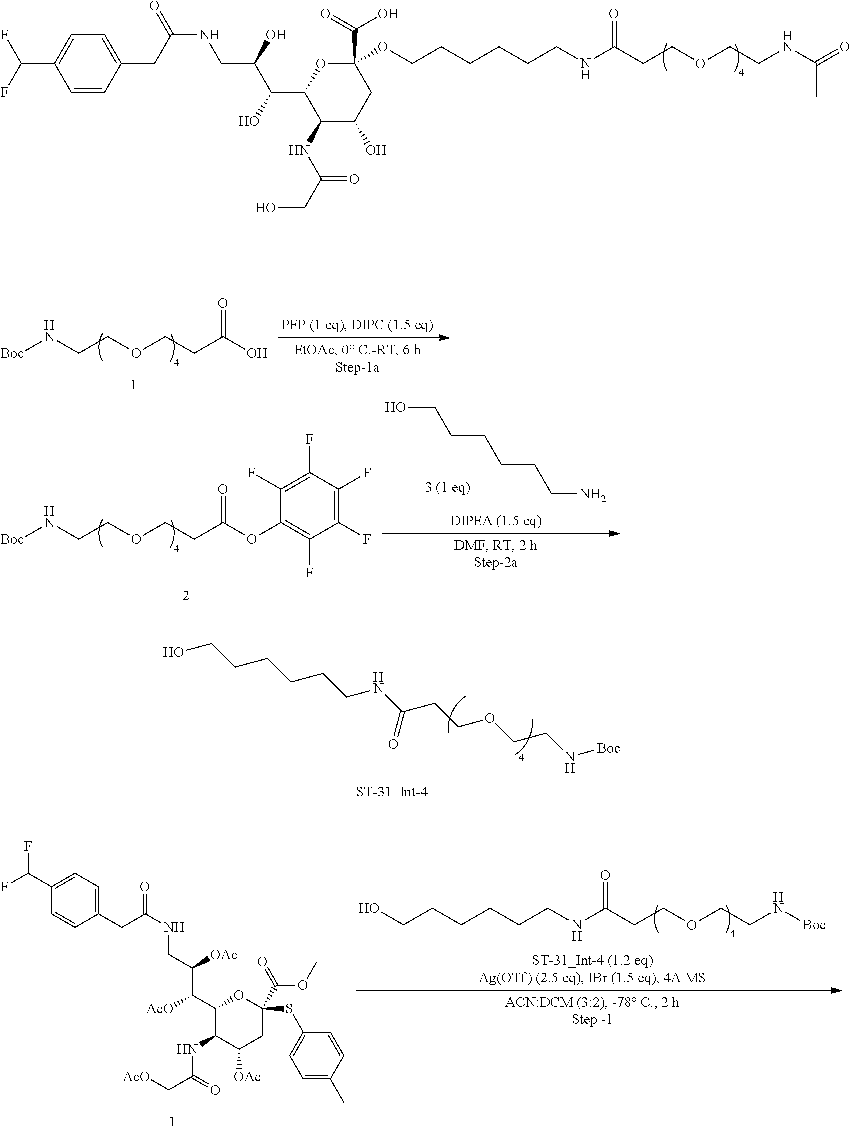

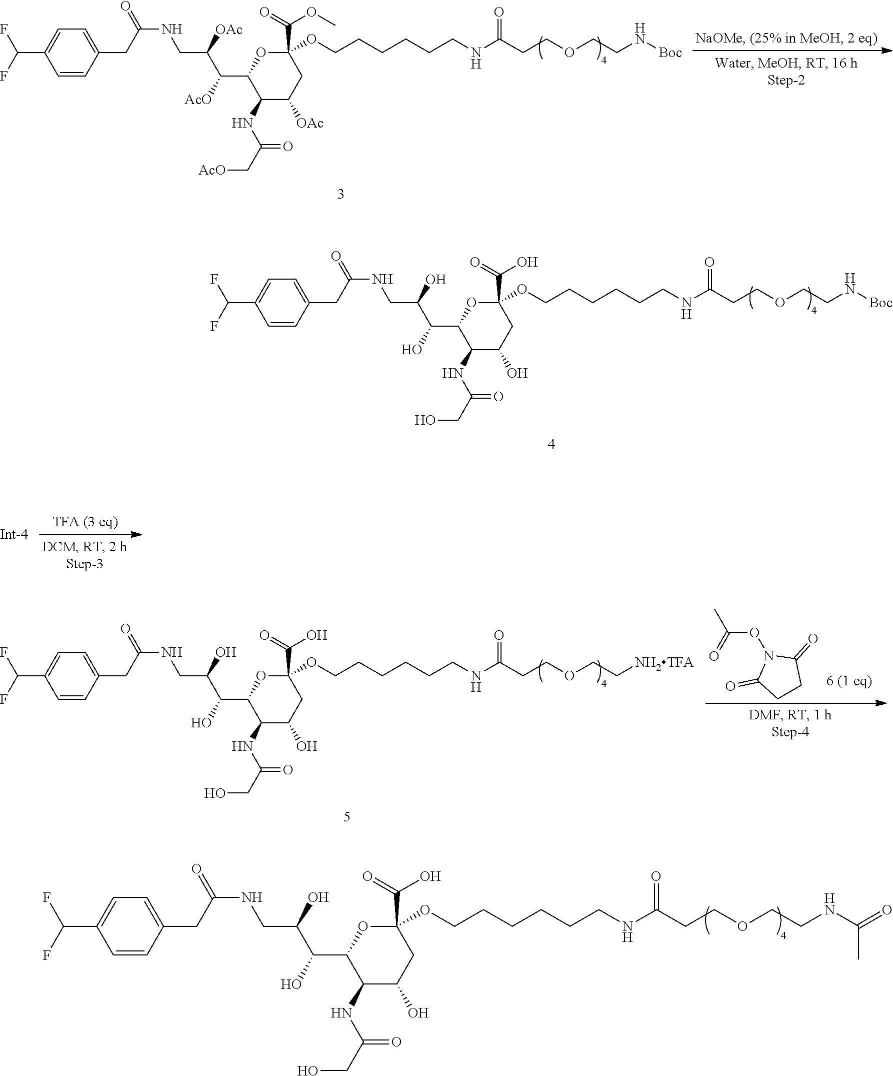

### Synthesis ofperfluorophenyl 2,2-dimethyl-4-oxo-3,8,11,14,17-pentaoxa-5-azaicosan-20-oate (2)

To a stirred solution of 2,2-dimethyl-4-oxo-3,8,11,14,17-pentaoxa-5-azaicosan-20-oic acid (1) (5 g, 13.6 mmol) and penta-fluoro phenol (2.5 g, 13.6 mmol) in Ethyl acetate (50 mL) was added di-iso-propyl carbodimide (2.58 g, 20.4 mmol) dropwise at 0° C. and the reaction mixture was stirred at room temperature for 6 h. After completion, the reaction mixture was filtered, filtrate was concentrated, the crude residue was purified by Silica gel column chromatography by using Ethyl acetate and Hexane as eluents to afford perfluorophenyl 2,2-dimethyl-4-oxo-3,8,11,14,17-pentaoxa-5-azaicosan-20-oate (2) as off-white solid, Yield: 5 g, 69.44%; LCMS (ESI±) m/z 533.65 [M+H]⁺.

### Synthesis of tert-butyl (22-hydroxy-15-oxo-3,6,9,12-tetraoxa-16-azadocosyl) carbamate (ST-31_Int-4)

To a stirred solution of perfluorophenyl 2,2-dimethyl-4-oxo-3,8,11,14,17-pentaoxa-5-azaicosan-20-oate (2) (4 g, 7.52 mmol) and 6-aminohexan-1-ol (3) (0.88 g, 7.52 mmol) in N,N-dimethylformamide (10 mL) was added diisopropyl ethylamine (2.08 ml, 11.29 mmol) at 0° C. The resulting reaction mixture was stirred at room temperature for 2 h. After completion, the reaction mixture was concentrated and purified by column chromatography by using Ethyl acetate and Hexane as eluents to afford tert-butyl (22-hydroxy-15-oxo-3,6,9,12-tetraoxa-16-azadocosyl) carbamate (ST-31_Int-4) as white solid, Yield: 2.4 g, 70.58%; LCMS (ESI±) m/z 465.5 [M+H]⁺.

### Synthesis of (1R,2R)-1-((2R,3R,4S,6R)-4-acetoxy-3-(2-acetoxyacetamido)-6-((2,2-dimethyl-4,20-dioxo-3,8,11,14,17-pentaoxa-5,21-diazaheptacosan-27-yl)oxy)-6-(methoxycarbonyl)tetrahydro-2H-pyran-2-yl)-3-(2-(4-(difluoromethyl)phenyl)acetamido)propane-1,2-diyl diacetate (3)

To a stirred solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-(difluoromethyl)phenyl)acetamido)propane-1,2-diyl diacetate (1) (2 g, 2.56 mmol), tert-butyl (22-hydroxy-15-oxo-3,6,9,12-tetraoxa-16-azadocosyl)carbamate (ST-31_Int-4) (1.43 g, 3.07 mmol) and Silver (1+) trifluoro methane sulfonate (AgOTf) (1.64 g, 6.40 mmol) in anhydrous dichloromethane (12 mL) and anhydrous acetonitrile (24 mL) was added activated 4 Å powdered molecular sieves (5 g) and the reaction mixture was stirred at room temperature for 1 h under nitrogen atmosphere. Then the solution was cooled to −78° C., iodine monobromide (0.79 g, 3.84 mmol) dissolved in dichloromethane (4 mL) was added dropwise, and the resulting mixture was stirred at same temperature for 2 h. After completion, the reaction mixture was quenched by addition of triethylamine (5 mL) and allowed to stir at room temperature for 1 h. Then the reaction mixture was filtered, the filtrate was washed with aqueous sodium thiosulfate solution, dried over sodium sulfate, filtered, and concentrated under reduced pressure. The crude residue obtained was purified by C18 reverse phase column chromatography using water: acetonitrile (+0.05% TFA) as eluents to afford the desired product (1R,2R)-1-((2R,3R,4S,6R)-4-acetoxy-3-(2-acetoxyacetamido)-6-((2,2-dimethyl-4,20-dioxo-3,8,11,14,17-pentaoxa-5,21-diazaheptacosan-27-yl)oxy)-6-(methoxycarbonyl)tetrahydro-2H-pyran-2-yl)-3-(2-(4-(difluoromethyl)phenyl)acetamido)propane-1,2-diyl diacetate (3) as a pale yellow solid, Yield: 1.1 g, 38%; LCMS (ESI+) m/z 1122.6 [M+H]⁺.

### Synthesis of (2R,4S,5R,6R)-6-((1R,2R)-3-(2-(4-(difluoro methyl) phenyl) acetamido)-1,2-dihydroxypropyl)-2-((2,2-dimethyl-4,20-dioxo-3,8,11,14,17-pentaoxa-5,21-diazaheptacosan-27-yl) oxy)-4-hydroxy-5-(2-hydroxyacetamido) tetrahydro-2H-pyran-2-carboxylic acid (4)

To a stirred solution of (1R,2R)-1-((2R,3R,4S,6R)-4-acetoxy-3-(2-acetoxyacetamido)-6-((2,2-dimethyl-4,20-dioxo-3,8,11,14,17-pentaoxa-5,21-diazaheptacosan-27-yl)oxy)-6-(methoxycarbonyl)tetrahydro-2H-pyran-2-yl)-3-(2-(4-(difluoromethyl)phenyl)acetamido)propane-1,2-diyl diacetate (3) (1.1 g, 0.98 mmol) in methanol (10 mL) and water (3 mL) was added NaOMe (25% in MeOH) (0.42 mL, 1.96 mmol) dropwise at 0° C. The resulting reaction mixture was stirred at room temperature for 16 h. After completion, the reaction mixture was treated with acidic (Dowex 50, H⁺) to maintain pH˜6 and the suspension was filtered. The filtrate was concentrated under reduced pressure and the crude was purified by C18 reverse phase using water: acetonitrile (+0.05% TFA) as eluents to afford the desired product (2R,4S,5R,6R)-6-((1R,2R)-3-(2-(4-(difluoromethyl)phenyl)acetamido)-1,2-dihydroxypropyl)-2-((2,2-dimethyl-4,20-dioxo-3,8,11,14,17-pentaoxa-5,21-diazaheptacosan-27-yl)oxy)-4-hydroxy-5-(2-hydroxyacetamido)tetrahydro-2H-pyran-2-carboxylic acid (4) as off white solid, Yield: 60 mg, 65%; LCMS (ESI+) m/z 940.7 [M+H]⁺.

### Synthesis of (2R,4S,5R,6R)-6-((1R,2R)-3-(2-(4-(difluoro methyl) phenyl) acetamido)-1,2-dihydroxypropyl)-4-hydroxy-5-(2-hydroxyacetamido)-2-((1,1,1-trifluoro-2,18-dioxo-6,9,12,15-tetraoxa-314,19-diazapentacosan-25-yl) oxy) tetrahydro-2H-pyran-2-carboxylic acid (5)

To a stirred solution of (2R,4S,5R,6R)-6-((1R,2R)-3-(2-(4-(difluoromethyl)phenyl)acetamido)-1,2-dihydroxypropyl)-2-((2,2-dimethyl-4,20-dioxo-3,8,11,14,17-pentaoxa-5,21-diazaheptacosan-27-yl)oxy)-4-hydroxy-5-(2-hydroxyacetamido)tetrahydro-2H-pyran-2-carboxylic acid (4) (60 mg, 0.64 mmol) in anhydrous dichloromethane (6 mL) was added trifluoro acetic acid (0.03 mL) dropwise at 0° C. and the resulting reaction mixture was stirred at room temperature for 3 h. After completion, the solvent was concentrated to get crude which was purified by prep-HPLC using acetonitrile and water (+0.05% TFA) as eluents to afford the desired product (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((1,1,1-trifluoro-2,18-dioxo-6,9,12,15-tetraoxa-314,19-diazapentacosan-25-yl)oxy)tetrahydro-2H-pyran-2-carboxylic acid (5) as yellow solid, Yield: 290 mg, 54%; LCMS (ESI+) m/z 839.7 [M+H]⁺.

### Synthesis of (2R,4S,5R,6R)-6-((1R,2R)-3-(2-(4-(difluoro methyl) phenyl) acetamido)-1,2-dihydroxypropyl)-2-((2,18-dioxo-6,9,12,15-tetraoxa-3,19-diazapentacosan-25-yl) oxy)-4-hydroxy-5-(2-hydroxyacetamido) tetrahydro-2H-pyran-2-carboxylic acid (256)

To a stirred solution of (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((1,1,1-trifluoro-2,18-dioxo-6,9,12,15-tetraoxa-314,19-diazapentacosan-25-yl)oxy)tetrahydro-2H-pyran-2-carboxylic acid (5) (120 mg, 0.14 mmol) in DMF (1.2 mL) were added 2,5-dioxopyrrolidin-1-yl acetate (6) (22 mg, 0.14 mmol), triethylamine (0.03 ml, 0.21 mmol) at 0° C., The resulting reaction mixture was stirred at the same temperature for 1 h. After completion, the reaction mixture was purified by prep-HPLC using acetonitrile and water (+0.05% TFA) as eluents to afford the desired product (2R,4S,5R,6R)-6-((1R,2R)-3-(2-(4-(difluoromethyl)phenyl)acetamido)-1,2-dihydroxypropyl)-2-((2,18-dioxo-6,9,12,15-tetraoxa-3,19-diazapentacosan-25-yl)oxy)-4-hydroxy-5-(2-hydroxyacetamido)tetrahydro-2H-pyran-2-carboxylic acid (256) as a white solid, Yield: 75 mg, 60.9%; LCMS (ESI+) m/z 881.8 [M+H]⁺.

¹H NMR (400 MHz, MeOD) δ 7.49 (d, J=8 Hz, 2H), 7.43 (d, J=8 Hz, 2H), 6.74 (t, J=56 Hz, 1H), 4.02 (s, 2H), 3.90-3.78 (m, 3H), 3.77-3.62 (m, 21H), 3.55-3.15 (m, 8H), 2.74-2.70 (m, 1H), 2.43 (t, J=6 Hz, 2H), 1.95 (s, 3H), 1.77-1.71 (m, 1H), 1.73-1.577 (m, 10H).

### (2R,4S,5R,6R)-6-((1R,2R)-3-(2-(4-(difluoro methyl) phenyl)acetamido)-1,2-dihydroxypropyl)-2-((1-(2,5-dioxo-2,5-dihydro-1H-pyrrol-1-yl)-3,19-dioxo-7,10,13,16-tetraoxa-4,20-diazahexacosan-26-yl)oxy)-4-hydroxy-5-(2-hydroxyacetamido)tetrahydro-2H-pyran-2-carboxylic acid (Cpd. 256M)

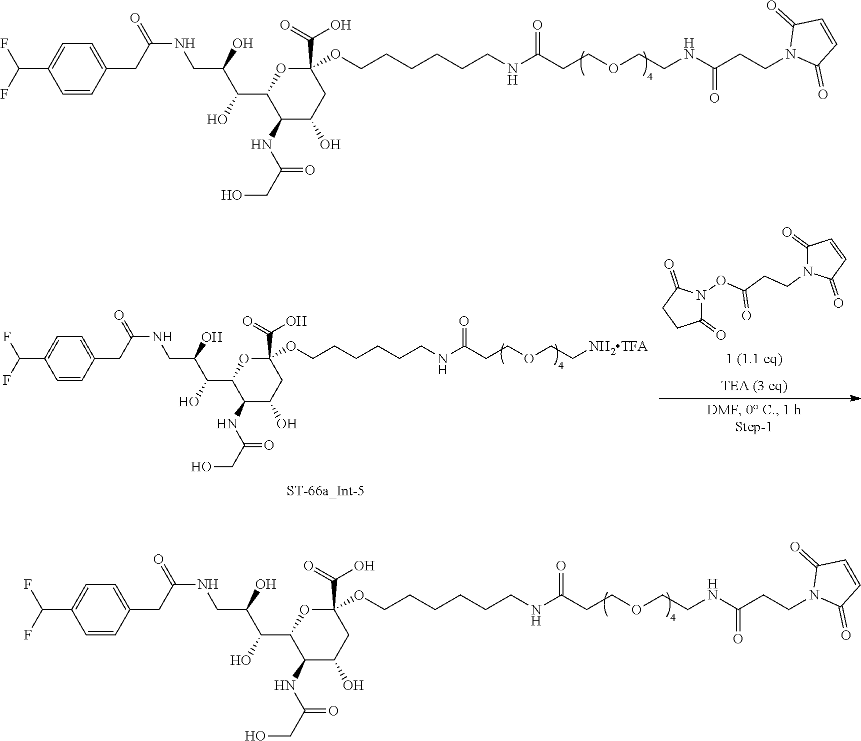

### Synthesis of (2R,4S,5R,6R)-6-((1R,2R)-3-(2-(4-(difluoromethyl)phenyl)acetamido)-1,2-dihydroxypropyl)-2-((1-(2,5-dioxo-2,5-dihydro-1H-pyrrol-1-yl)-3,19-dioxo-7,10,13,16-tetraoxa-4,20-diazahexacosan-26-yl)oxy)-4-hydroxy-5-(2-hydroxyacetamido)tetrahydro-2H-pyran-2-carboxylic acid (256M)

To a stirred solution of (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((1,1,1-trifluoro-2,18-dioxo-6,9,12,15-tetraoxa-314,19-diazapentacosan-25-yl)oxy)tetrahydro-2H-pyran-2-carboxylic acid (256_Int-5) (120 mg, 0.14 mmol) in DMF (1.2 mL) were added 2,5-dioxopyrrolidin-1-yl 3-(2,5-dioxo-2,5-dihydro-1H-pyrrol-1-yl)propanoate (1) (41.8 mg, 0.16 mmol), triethylamine (0.05 mL, 0.429 mmol) at 0° C. The resulting reaction mixture was stirred at the same temperature for 1 h. After completion, the reaction mixture was purified by prep-HPLC using acetonitrile and water (+0.05% TFA) as eluents to afford the desired product (2R,4S,5R,6R)-6-((1R,2R)-3-(2-(4-(difluoromethyl)phenyl)acetamido)-1,2-dihydroxypropyl)-2-((1-(2,5-dioxo-2,5-dihydro-1H-pyrrol-1-yl)-3,19-dioxo-7,10,13,16-tetraoxa-4,20-diazahexacosan-26-yl)oxy)-4-hydroxy-5-(2-hydroxyacetamido)tetrahydro-2H-pyran-2-carboxylic acid (256M) as white solid, Yield: 63 mg, 44%; LCMS (ESI+) m/z 991.9 [M+H]⁺. ¹H NMR (400 MHz, MeOD) δ 7.47 (dd, J=23.2, 8.4 Hz, 4H), 6.83 (s, 2H), 6.75 (t, J=56 Hz, 1H), 4.03 (s, 2H), 3.83-3.69 (m, 10H), 3.65-3.18 (m, 22H), 2.74-2.70 (m, 1H), 2.50-2.42 (m, 4H), 2.05-1.36 (m, 9H).

### (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((5,21,37-trioxo-1-((3aR,4R,6aS)-2-oxohexahydro-1H-thieno[3,4-d]imidazol-4-yl)-9,12,15,18,25,28,31,34-octaoxa-6,22,38-triazatetratetracontan-44-yl)oxy)tetrahydro-2H-pyran-2-carboxylic acid (Cpd. 256B)

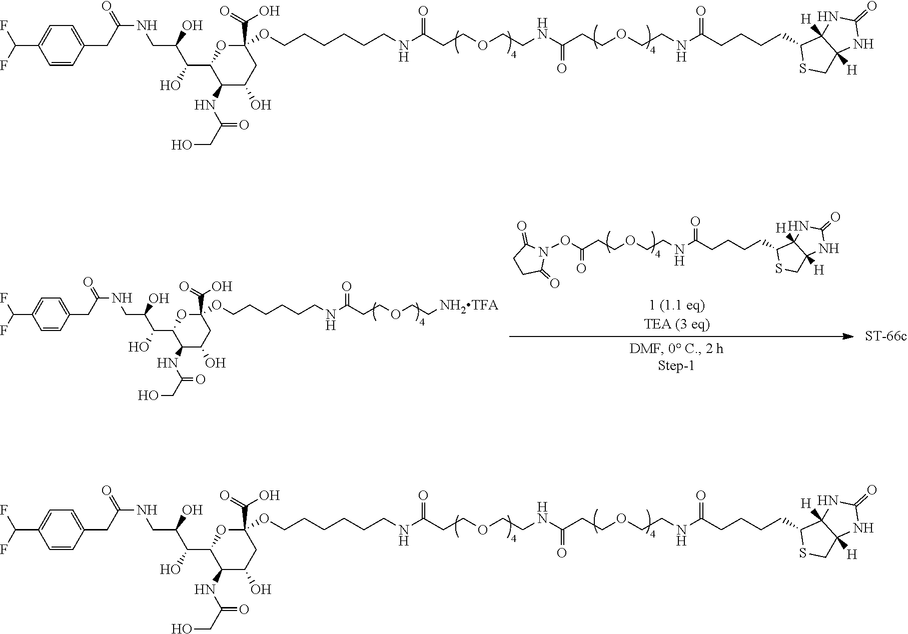

### Synthesis of (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((5,21,37-trioxo-1-((3aR,4R,6aS)-2-oxohexahydro-1H-thieno[3,4-d]imidazol-4-yl)-9,12,15,18,25,28,31,34-octaoxa-6,22,38-triazatetratetracontan-44-yl)oxy)tetrahydro-2H-pyran-2-carboxylic acid (256B)

To a stirred solution of (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((1,1,1-trifluoro-2,18-dioxo-6,9,12,15-tetraoxa-314,19-diazapentacosan-25-yl)oxy)tetrahydro-2H-pyran-2-carboxylic acid (256_Int-5) (50 mg, 0.05 mmol) in anhydrous N,N-dimethyl formamide (1 mL) were added 2,5-dioxopyrrolidin-1-yl 17-oxo-21-((3aR,4R,6aS)-2-oxohexahydro-1H-thieno[3,4-d]imidazol-4-yl)-4,7,10,13-tetraoxa-16-azahenicosanoate (1) (31 mg, 0.05 mmol) and triethylamine (0.02 ml, 0.16 mmol) at 0° C. The resulting reaction mixture was stirred at 0° C. for 2 h. After completion, the reaction mixture was purified by prep-HPLC purification using acetonitrile and water (+0.05%, TFA) as eluents to afford the desired product (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((5,21,37-trioxo-1-((3aR,4R,6aS)-2-oxohexahydro-1H-thieno[3,4-d]imidazol-4-yl)-9,12,15,18,25,28,31,34-octaoxa-6,22,38-triazatetratetracontan-44-yl)oxy)tetrahydro-2H-pyran-2-carboxylic acid (256B) as white solid, Yield: 50 mg, 72%; LCMS (ESI+) m/z 1313.4 [M+H]⁺. ¹H NMR (400 MHz, MeOD) δ 7.48 (dd, J=22.8, 8 Hz, 4H), 6.77 (t, J=56 Hz, 1H), 4.53-4.30 (m, 2H), 4.04 (s, 2H), 3.84-3.57 (m, 23H), 3.55-3.37 (m, 24H), 3.34-3.19 (m, 4H), 2.94-2.72 (m, 3H), 2.50-2.44 (m, 4H), 2.25 (t, J=7.2 Hz, 2H), 1.76-1.38 (m, 16H).

### (2R,4S,5R,6R)-6-((1R,2R)-3-(2-(4-(difluoro methyl) phenyl) acetamido)-1,2-dihydroxypropyl)-2-((2,19-dioxo-6,9,12,15-tetraoxa-3,18-diazapentacosan-25-yl)oxy)-4-hydroxy-5-(2-hydroxyacetamido)tetrahydro-2H-pyran-2-carboxylic acid (Cpd. 257)

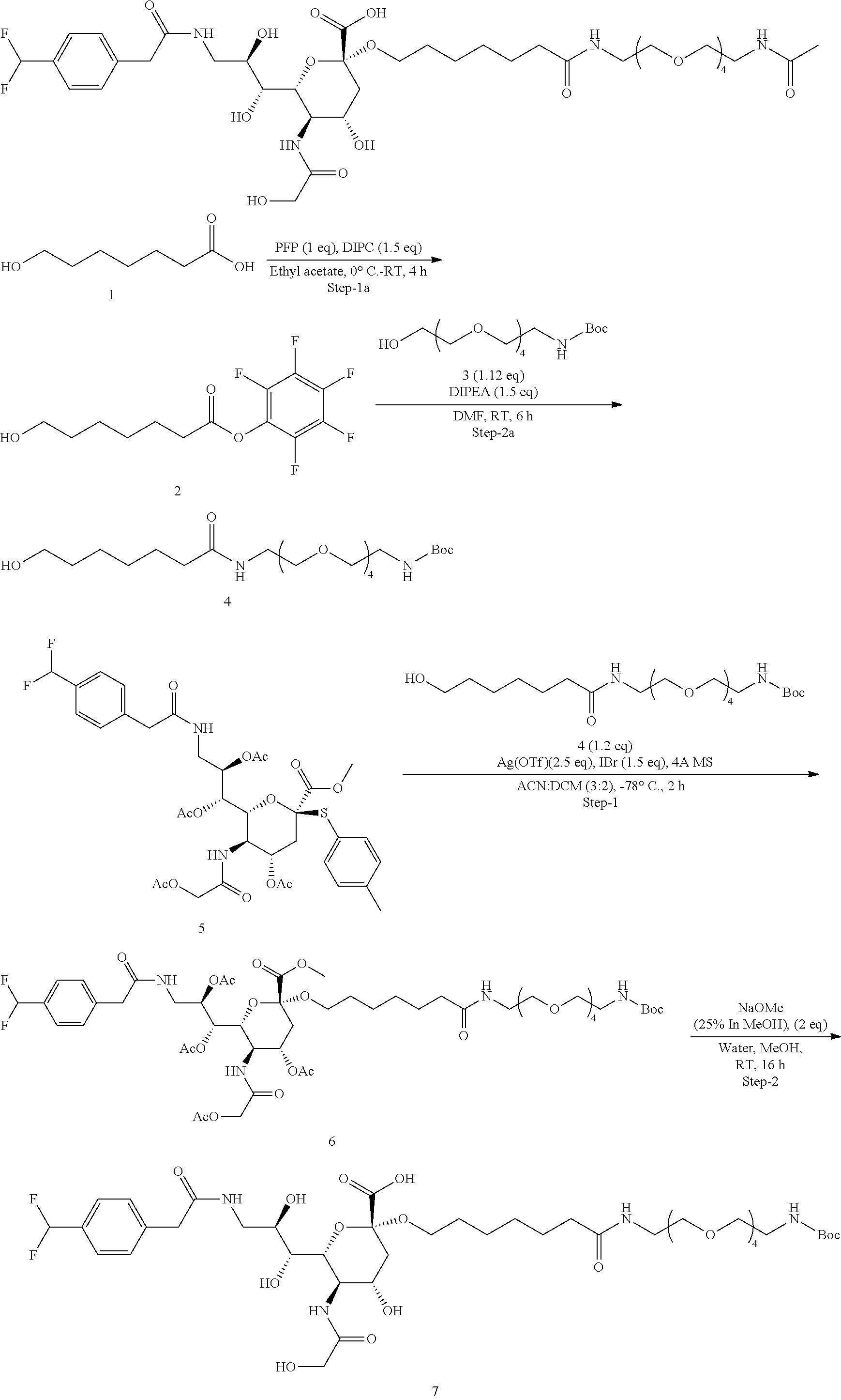

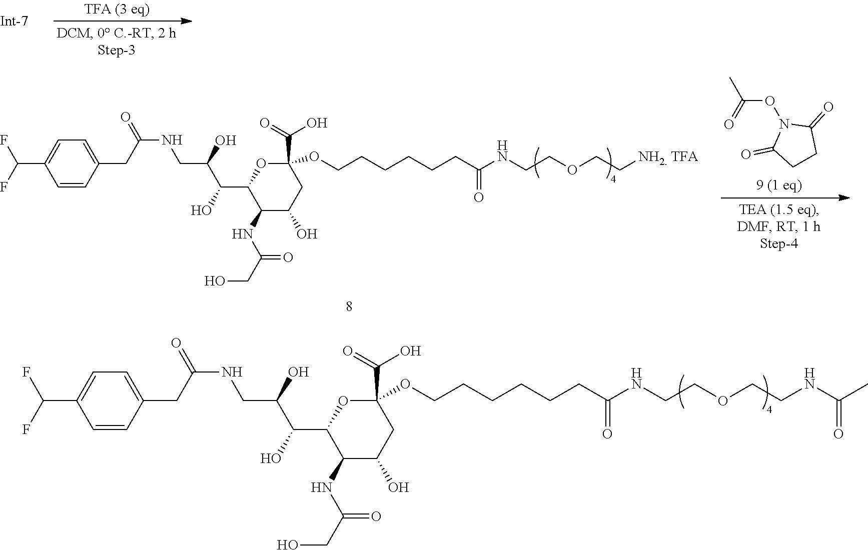

### Synthesis of perfluorophenyl 7-hydroxyheptanoate (2)

To a stirred solution of 7-hydroxyheptanoic acid (1) (5 g, 34 mmol) in EtOAc (50 mL) was added 2,3,4,5,6-pentafluorophenol (6.25 g, 34 mmol), followed by dropwise addition of N, N′-Di-isopropyl carbodiimide (6.43 g, 51 mmol) at 0° C. The resulting reaction mixture was stirred for 4 h at the same temperature. After completion, the reaction mixture was filtered, the residue was washed with ethyl acetate, and the filtrate was concentrated under reduced pressure to afford of the desired product perfluorophenyl 7-hydroxyheptanoate (2), which was used as such for the next step, Yield: 7 g, 65%.

### Synthesis of tert-butyl (22-hydroxy-16-oxo-3,6,9,12-tetraoxa-15-azadocosyl) carbamate (4)

To a stirred solution of perfluorophenyl 7-hydroxyheptanoate (2) (7 g, 22 mmol) in DMF (70 mL) were added tert-butyl (14-amino-3,6,9,12-tetraoxatetradecyl)carbamate (4) (7.5 g, 22 mmol), DIPEA (6 mL, 33 mmol) at 0° C. and the reaction mixture was stirred at the same temperature for next 2 h. After completion, the reaction mixture was purified by C18 reverse phase column chromatography using water and acetonitrile (+0.05% TFA) as eluents to afford the desired product tert-butyl (22-hydroxy-16-oxo-3,6,9,12-tetraoxa-15-azadocosyl) carbamate (4) as off-white solid. Yield: 7 g, 67%; LCMS (ESI+) m/z 465.5 [M+H]⁺

### Synthesis of (1R,2R)-1-((2R,3R,4S,6R)-4-acetoxy-3-(2-acetoxyacetamido)-6-((2,2-dimethyl-4,21-dioxo-3,8,11,14,17-pentaoxa-5,20-diazaheptacosan-27-yl)oxy)-6-(methoxycarbonyl)tetrahydro-2H-pyran-2-yl)-3-(2-(4-(difluoromethyl)phenyl)acetamido)propane-1,2-diyl diacetate (6)

To a stirred solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-(difluoromethyl)phenyl)acetamido)propane-1,2-diyl diacetate (5) (2 g, 2.56 mmol), tert-butyl (22-hydroxy-16-oxo-3,6,9,12-tetraoxa-15-azadocosyl)carbamate (4), Silver (1+) trifluoro methane sulfonate (AgOTf) (1.64 g, 6.40 mmol) in anhydrous dichloromethane (12 mL) and anhydrous acetonitrile (24 mL) was added activated 4 Å powdered molecular sieves (5 g) and the reaction mixture was stirred at room temperature for 1 h under nitrogen atmosphere. The solution was cooled to −78° C., iodine monobromide (0.79 g, 3.84 mmol) dissolved in dichloromethane (4 mL) was added dropwise and the resulting mixture was stirred at the same temperature for 2 h. After completion, the reaction mixture was quenched with triethylamine (5 mL) and stirred at room temperature. The reaction mixture was filtered, the filtrate was washed with 10% aqueous sodium thiosulfate solution, dried over sodium sulfate, filtered, concentrated under reduced pressure. The crude residue was purified by C18 reverse phase column chromatography using water and acetonitrile (+0.05% TFA) as eluents to afford the desired product (1R,2R)-1-((2R,3R,4S,6R)-4-acetoxy-3-(2-acetoxyacetamido)-6-((2,2-dimethyl-4,21-dioxo-3,8,11,14,17-pentaoxa-5,20-diazaheptacosan-27-yl)oxy)-6-(methoxycarbonyl)tetrahydro-2H-pyran-2-yl)-3-(2-(4-(difluoromethyl)phenyl)acetamido)propane-1,2-diyl diacetate (6) as a pale yellow solid, Yield: 1.1 g, 38%; LCMS (ESI+) m/z 1122.6 [M+H]⁺.

### Synthesis of (2R,4S,5R,6R)-6-((1R,2R)-3-(2-(4-(difluoro methyl) phenyl) acetamido)-1,2-dihydroxypropyl)-2-((2,2-dimethyl-4,21-dioxo-3,8,11,14,17-pentaoxa-5,20-diazaheptacosan-27-yl)oxy)-4-hydroxy-5-(2-hydroxyacetamido)tetrahydro-2H-pyran-2-carboxylic acid (7)

To a stirred solution (1R,2R)-1-((2R,3R,4S,6R)-4-acetoxy-3-(2-acetoxyacetamido)-6-((2,2-dimethyl-4,21-dioxo-3,8,11,14,17-pentaoxa-5,20-diazaheptacosan-27-yl)oxy)-6-(methoxycarbonyl)tetrahydro-2H-pyran-2-yl)-3-(2-(4-(difluoromethyl)phenyl)acetamido)propane-1,2-diyl diacetate (6) (1.1 g, 0.98 mmol) in methanol (10 mL) and water (3 mL) was added NaOMe (25% in MeOH) (0.42 mL, 1.96 mmol) dropwise at 0° C. The resulting reaction mixture was stirred at room temperature for 16 h. After completion, the reaction mixture was treated with acidic resin (Dowex 50, H⁺) to maintain pH˜6 and the suspension was filtered with methanol washing. The filtrate was concentrated under reduced pressure and the crude residue obtained was purified by C18 reverse phase column chromatography using water and acetonitrile (+0.05% TFA) as eluents to afford the desired product (2R,4S,5R,6R)-6-((1R,2R)-3-(2-(4-(difluoromethyl)phenyl)acetamido)-1,2-dihydroxypropyl)-2-((2,2-dimethyl-4,21-dioxo-3,8,11,14,17-pentaoxa-5,20-diazaheptacosan-27-yl)oxy)-4-hydroxy-5-(2-hydroxyacetamido)tetrahydro-2H-pyran-2-carboxylic acid (7) as an off white solid, Yield: 0.620 g, 67%; LCMS (ESI+) m/z 940.7 [M+H]⁺.

### Synthesis of (2R,4S,5R,6R)-2-((1-amino-16-oxo-3,6,9,12-tetraoxa-15-azadocosan-22-yl) oxy)-6-((1R,2R)-3-(2-(4-(difluoro methyl) phenyl) acetamido)-1,2-dihydroxypropyl)-4-hydroxy-5-(2-hydroxyacetamido) tetrahydro-2H-pyran-2-carboxylic acid (8)

To a stirred solution of (2R,4S,5R,6R)-6-((1R,2R)-3-(2-(4-(difluoromethyl)phenyl)acetamido)-1,2-dihydroxypropyl)-2-((2,2-dimethyl-4,21-dioxo-3,8,11,14,17-pentaoxa-5,20-diazaheptacosan-27-yl)oxy)-4-hydroxy-5-(2-hydroxyacetamido)tetrahydro-2H-pyran-2-carboxylic acid (7) (0.62 g, 0.66 mmol) in anhydrous dichloromethane (6.2 mL) was added trifluoroacetic acid (1.8 mL) dropwise at 0° C. and the resulting reaction mixture was stirred at room temperature for 3 h. After completion, the reaction mixture was concentrated and the crude residue obtained was purified by prep-HPLC (15-80% acetonitrile in water, +0.05% TFA) to afford the desired product (2R,4S,5R,6R)-2-((1-amino-16-oxo-3,6,9,12-tetraoxa-15-azadocosan-22-yl)oxy)-6-((1R,2R)-3-(2-(4-(difluoromethyl)phenyl)acetamido)-1,2-dihydroxypropyl)-4-hydroxy-5-(2-hydroxyacetamido)tetrahydro-2H-pyran-2-carboxylic acid (8) as yellow solid, Yield: 295 mg, 53%; LCMS (ESI+) m/z 840.5 [M+H]⁺.

### Synthesis of (2R,4S,5R,6R)-6-((1R,2R)-3-(2-(4-(difluoro methyl) phenyl) acetamido)-1,2-dihydroxypropyl)-2-((2,19-dioxo-6,9,12,15-tetraoxa-3,18-diazapentacosan-25-yl) oxy)-4-hydroxy-5-(2-hydroxyacetamido) tetrahydro-2H-pyran-2-carboxylic acid (257)

To a stirred solution of (2R,4S,5R,6R)-2-((1-amino-16-oxo-3,6,9,12-tetraoxa-15-azadocosan-22-yl)oxy)-6-((1R,2R)-3-(2-(4-(difluoromethyl)phenyl)acetamido)-1,2-dihydroxypropyl)-4-hydroxy-5-(2-hydroxyacetamido)tetrahydro-2H-pyran-2-carboxylic acid (8) (0.12 g, 0.14 mmol) in DMF (1.2 mL) and 2,5-dioxopyrrolidin-1-yl acetate (9) (22 mg, 0.14 mmol) was added triethylamine (0.03 ml, 0.21 mmol) at 0° C. The resulting reaction mixture was stirred at the same temperature for 1 h. After completion, the reaction mixture was purified by prep-HPLC to afford the desired product (2R,4S,5R,6R)-6-((1R,2R)-3-(2-(4-(difluoromethyl)phenyl)acetamido)-1,2-dihydroxypropyl)-2-((2,19-dioxo-6,9,12,15-tetraoxa-3,18-diazapentacosan-25-yl)oxy)-4-hydroxy-5-(2-hydroxyacetamido)tetrahydro-2H-pyran-2-carboxylic acid (257) as white solid, Yield: 10 mg, 8.7%; LCMS (ESI+) m/z 881.7 [M+H]⁺. ¹H NMR (400 MHz, MeOD) δ 7.51-7.43 (m, 4H), 6.75 (t, J=56 Hz, 1H), 4.04 (s, 2H), 4.03-3.76 (m, 3H), 3.75-3.53 (m, 21H), 3.44-3.24 (m, 7H), 2.74-2.70 (m, 1H), 2.22-2.18 (m, 2H), 1.96 (s, 3H), 1.77-1.71 (m, 1H), 1.63-1.35 (m, 8H).

### (2R,4S,5R,6R)-6-((1R,2R)-3-(2-(4-(difluoro methyl) phenyl)acetamido)-1,2-dihydroxypropyl)-2-((1-(2,5-dioxo-2,5-dihydro-1H-pyrrol-1-yl)-3,20-dioxo-7,10,13,16-tetraoxa-4,19-diazahexacosan-26-yl)oxy)-4-hydroxy-5-(2-hydroxyacetamido)tetrahydro-2H-pyran-2-carboxylic acid (Cpd. 257M)

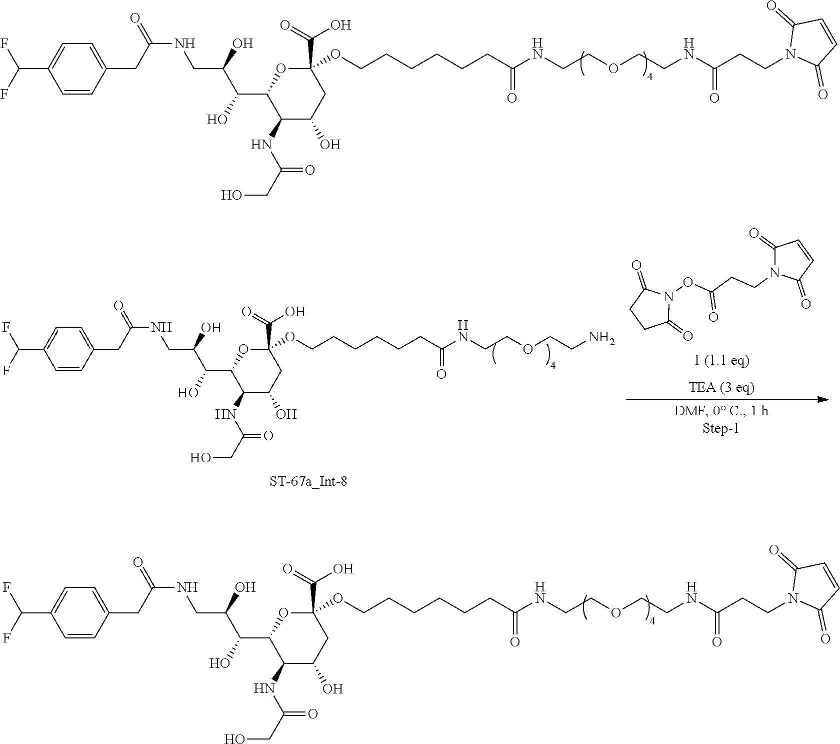

### Synthesis of (2R,4S,5R,6R)-6-((1R,2R)-3-(2-(4-(difluoromethyl)phenyl)acetamido)-1,2-dihydroxypropyl)-2-((1-(2,5-dioxo-2,5-dihydro-1H-pyrrol-1-yl)-3,20-dioxo-7,10,13,16-tetraoxa-4,19-diazahexacosan-26-yl)oxy)-4-hydroxy-5-(2-hydroxyacetamido)tetrahydro-2H-pyran-2-carboxylic acid (257M)

To a stirred solution of (2R,4S,5R,6R)-2-((1-amino-16-oxo-3,6,9,12-tetraoxa-15-azadocosan-22-yl)oxy)-6-((1R,2R)-3-(2-(4-(difluoromethyl)phenyl)acetamido)-1,2-dihydroxypropyl)-4-hydroxy-5-(2-hydroxyacetamido)tetrahydro-2H-pyran-2-carboxylic acid (257_Int-8) (120 mg, 0.14 mmol) in DMF (1.2 mL) were added 2,5-dioxopyrrolidin-1-yl 3-(2,5-dioxo-2,5-dihydro-1H-pyrrol-1-yl)propanoate (1) (41.8 mg, 0.157 mmol) and triethylamine (0.05 mL, 0.43 mmol) at 0° C. The resulting reaction mixture was stirred at the same temperature for 1 h. After completion, the reaction mixture was purified by prep-HPLC to afford the desired product (2R,4S,5R,6R)-6-((1R,2R)-3-(2-(4-(difluoromethyl)phenyl)acetamido)-1,2-dihydroxypropyl)-2-((1-(2,5-dioxo-2,5-dihydro-1H-pyrrol-1-yl)-3,20-dioxo-7,10,13,16-tetraoxa-4,19-diazahexacosan-26-yl)oxy)-4-hydroxy-5-(2-hydroxyacetamido)tetrahydro-2H-pyran-2-carboxylic acid (257M) as a white solid, Yield: 80 mg, 57%; LCMS (ESI+) m/z 991.9 [M+H]⁺.

¹H NMR (400 MHz, MeOD) δ 7.50-7.42 (m, 4H), 6.82 (s, 2H), 6.74 (t, J=56 Hz, 1H), 4.02 (s, 2H), 3.89-3.61 (m, 22H), 3.55-3.25 (m, 11H), 2.74-2.70 (m, 1H), 2.47 (t, J=6.8 Hz, 2H), 2.21-2.17 (m, 2H), 1.62-1.34 (m, 9H).

### (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((5,21,38-trioxo-1-((3aR,4R,6aS)-2-oxohexahydro-1H-thieno[3,4-d]imidazol-4-yl)-9,12,15,18,25,28,31,34-octaoxa-6,22,37-triazatetratetracontan-44-yl)oxy)tetrahydro-2H-pyran-2-carboxylic acid (Cpd. 257B)

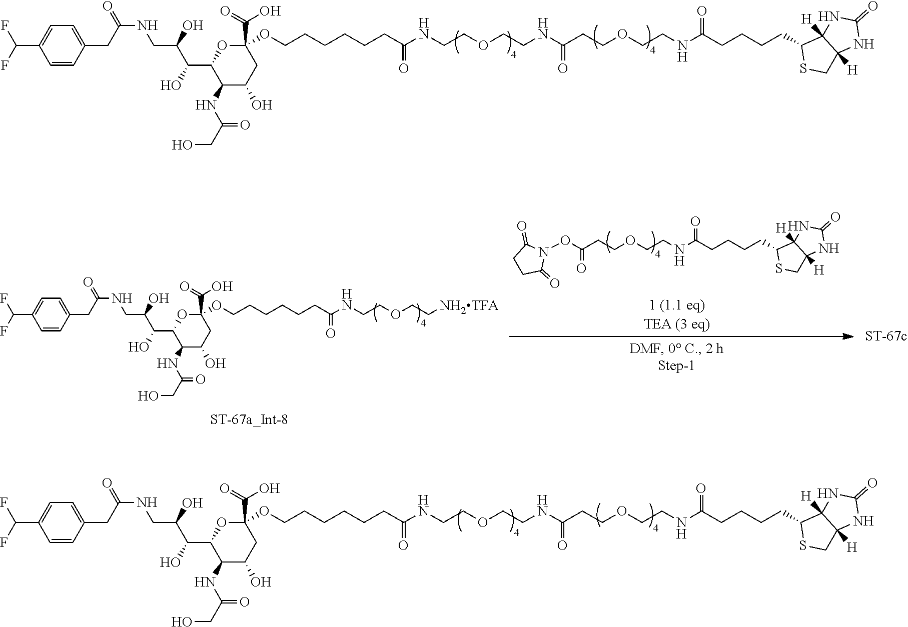

### Synthesis of (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((5,21,38-trioxo-1-((3aR,4R,6aS)-2-oxohexahydro-1H-thieno[3,4-d]imidazol-4-yl)-9,12,15,18,25,28,31,34-octaoxa-6,22,37-triazatetratetracontan-44-yl)oxy)tetrahydro-2H-pyran-2-carboxylic acid (257B)

To a stirred solution of (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((5,21,38-trioxo-1-((3aR,4R,6aS)-2-oxohexahydro-1H-thieno[3,4-d]imidazol-4-yl)-9,12,15,18,25,28,31,34-octaoxa-6,22,37-triazatetratetracontan-44-yl)oxy)tetrahydro-2H-pyran-2-carboxylic acid (257_Int-8) (50 mg, 0.05 mmol) in anhydrous N,N-dimethyl formamide (1 mL) were added 2,5-dioxopyrrolidin-1-yl 17-oxo-21-((3aR,4R,6aS)-2-oxohexahydro-1H-thieno[3,4-d]imidazol-4-yl)-4,7,10,13-tetraoxa-16-azahenicosanoate (1) (31 mg, 0.05 mmol) and triethylamine (0.02 ml, 0.16 mmol) at 0° C. The resulting reaction mixture was stirred at the same temperature for 2 h. After completion, the reaction mixture was purified by prep-HPLC using acetonitrile and water (+0.05%, TFA) as eluents to afford the desired product (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((5,21,38-trioxo-1-((3aR,4R,6aS)-2-oxohexahydro-1H-thieno[3,4-d]imidazol-4-yl)-9,12,15,18,25,28,31,34-octaoxa-6,22,37-triazatetratetracontan-44-yl)oxy)tetrahydro-2H-pyran-2-carboxylic acid (257B) as white solid, Yield: 30 mg, 43%; LCMS (ESI+) m/z 1313.4 [M+H]⁺. ¹H NMR (400 MHz, MeOD) δ 7.51-7.43 (m, 4H), 6.75 (t, J=56 Hz, 1H), 4.53-4.30 (m, 2H), 4.03 (s, 2H), 3.76-3.65 (m, 22H), 3.63-3.53 (m, 17H), 3.40-3.32 (m, 10H), 2.98-2.91 (m, 1H), 2.74-2.71 (m, 2H), 2.47 (t, J=6 Hz, 2H), 2.26-2.18 (m, 4H), 1.74-1.35 (m, 16H).

### (2R,4S,5R,6R)-6-((1R,2R)-3-(2-(4-(difluoromethyl)phenyl)acetamido)-1,2-dihydroxypropyl)-2-((6-((3,4-dioxo-2-((2-oxo-6,9,12,15-tetraoxa-3-azaheptadecan-17-yl)amino)cyclobut-1-en-1-yl)amino)hexyl)oxy)-4-hydroxy-5-(2-hydroxyacetamido)tetrahydro-2H-pyran-2-carboxylic acid (Cpd. 258)

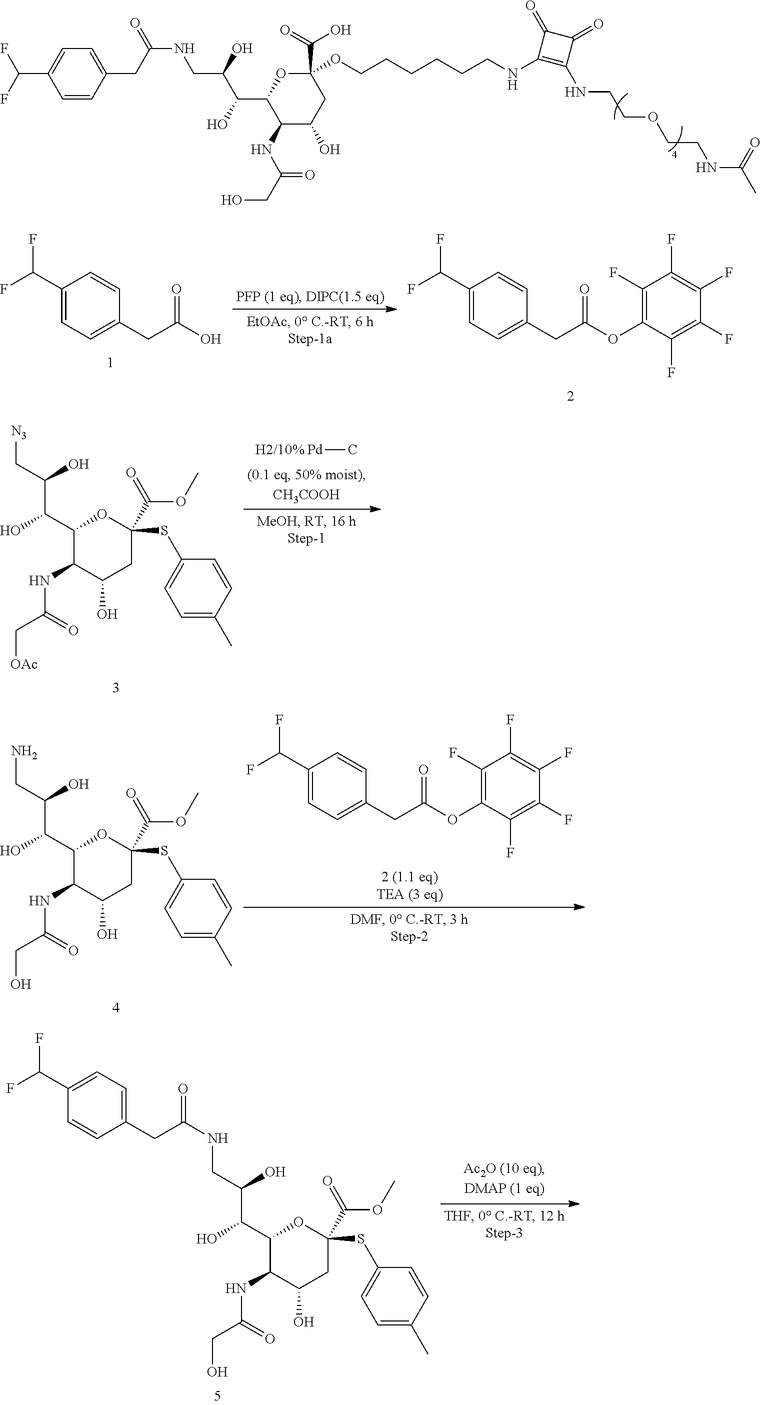

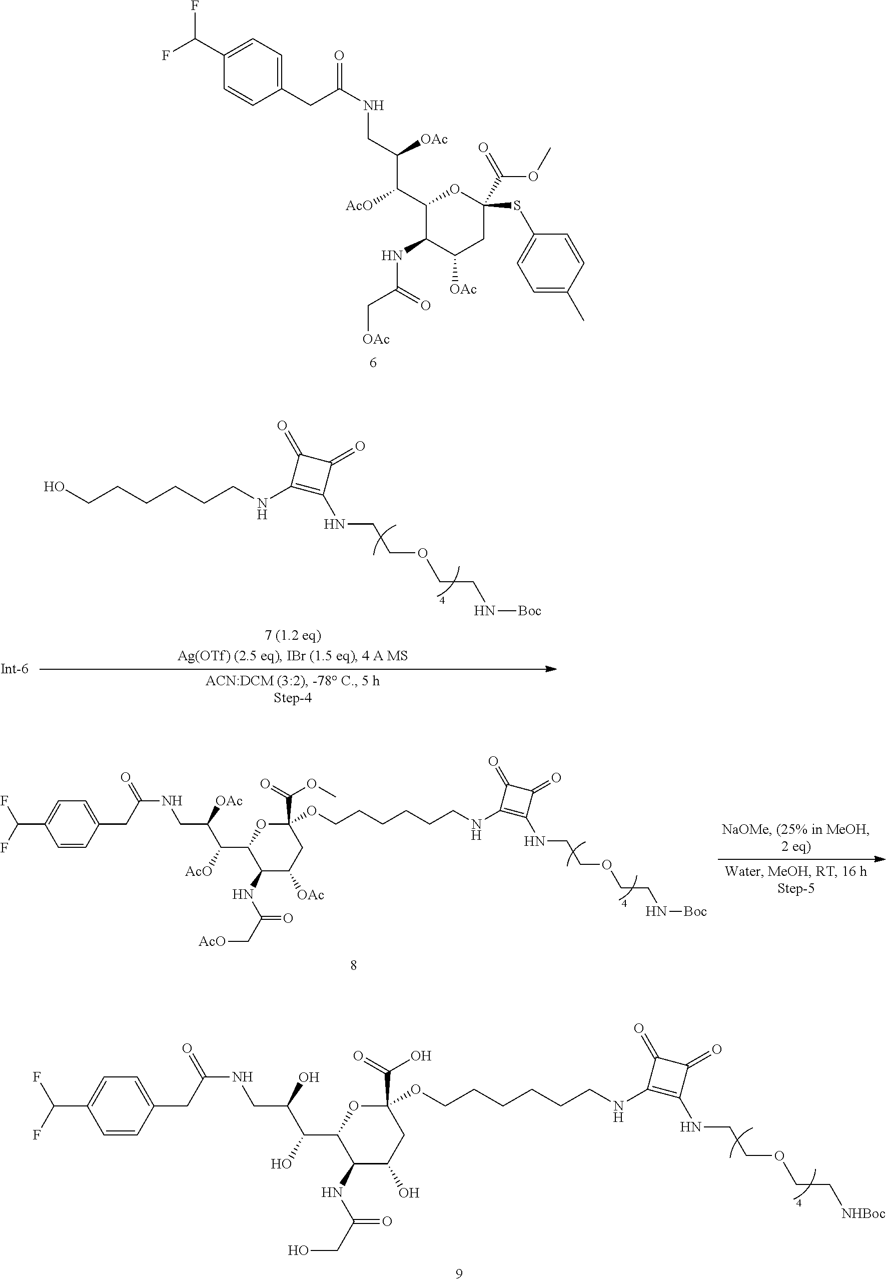

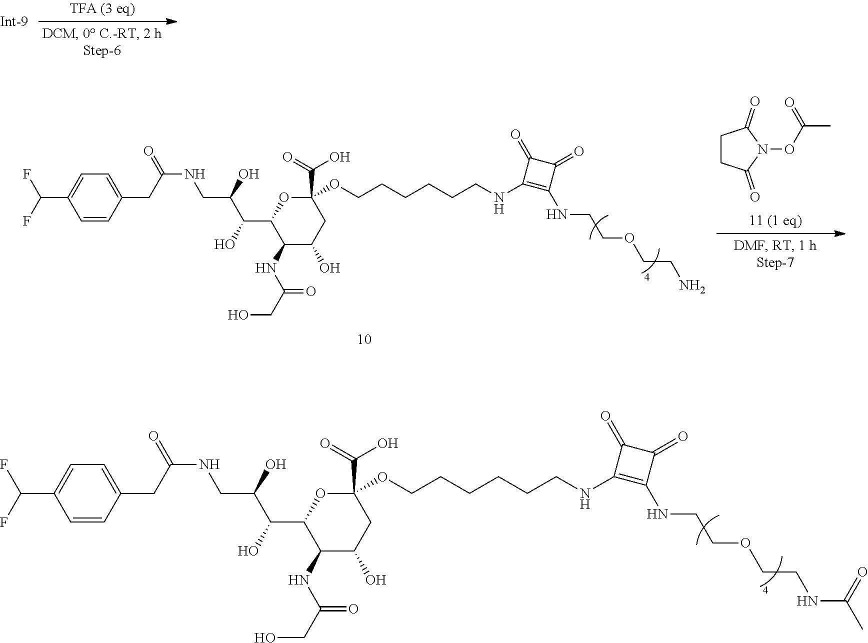

### Synthesis of perfluorophenyl 2-(4-(difluoro methyl) phenyl) acetate (2)

To a 2-(4-(difluoro methyl) phenyl) acetic acid (1) (5 g, 27 mmol) in EtOAc (50 mL) was added 2,3,4,5,6-pentafluorophenol (4.96 g, 27 mmol), followed by dropwise addition of N, N′-Di-isopropyl carbodiimide (5.1 g, 40.5 mmol) at 0° C. The resulting reaction mixture was stirred at 0° C. for 6 h. After completion, the reaction mixture was filtered, residue was washed with ethyl acetate. The filtrate was concentrated under reduced pressure to afford crude product perfluorophenyl 2-(4-(difluoromethyl)phenyl)acetate (2), which was directly used in the next step without further purification, Yield: 15 g (crude).

### Synthesis of methyl (2R,4S,5R,6R)-6-((1R,2R)-3-amino-1,2-dihydroxypropyl)-4-hydroxy-5-(2-hydroxyacetamido)-2-(p-tolylthio) tetrahydro-2H-pyran-2-carboxylate (4)

To a stirred solution of methyl (2R,4S,5R,6R)-5-(2-acetoxyacetamido)-6-((1R,2R)-3-azido-1,2-dihydroxypropyl)-4-hydroxy-2-(p-tolylthio)tetrahydro-2H-pyran-2-carboxylate (3) (17 g, 33.20 mmol) in methanol (170 mL) were added 10% Pd/C (50% moist) (5.2 g, 3.32 mmol), CH₃COOH (1 mL) and the reaction mixture was stirred at RT in presence of H₂ gas for 16 h. After completion, Pd/C was filtered, the filtrate was concentrated under reduced pressure to afford crude product methyl (2R,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (4), which was directly used in the next step without further purification, Yield: 15 g (crude).

### Synthesis of methyl (2R,4S,5R,6R)-6-((1R,2R)-3-(2-(4-(difluoro methyl) phenyl) acetamido)-1,2-dihydroxypropyl)-4-hydroxy-5-(2-hydroxyacetamido)-2-(p-tolylthio) tetrahydro-2H-pyran-2-carboxylate (5)

To a stirred solution of methyl (2R,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (4) (15 g, 33.75 mmol) in DMF (90 mL) were added perfluorophenyl 2-(4-(difluoromethyl)phenyl)acetate (2) (13.07 g, 37.12 mmol), triethylamine (15 mL, 101 mmol) dropwise at 0° C. The resulting reaction mixture was stirred at room temperature for 3 h. After completion, the reaction mixture was concentrated under reduced pressure to afford crude product methyl (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-(p-tolylthio)tetrahydro-2H-pyran-2-carboxylate (5), which was directly used in the next step without further purification, Yield: 17 g (crude).

### Synthesis of (1R,2R)-1-((2R,3R,4S,6R)-4-acetoxy-3-(2-acetoxyacetamido)-6-(methoxycarbonyl)-6-(p-tolylthio) tetrahydro-2H-pyran-2-yl)-3-(2-(4-(difluoro methyl) phenyl) acetamido) propane-1,2-diyl diacetate (6)

To a stirred solution of methyl (2R,4S,5R,6R)-6-((1R,2R)-3-(2-(4-(difluoro methyl)phenyl) acetamido)-1,2-dihydroxypropyl)-4-hydroxy-5-(2-hydroxyacetamido)-2-(p-tolylthio)tetrahydro-2H-pyran-2-carboxylate (5) (17 g, 27.75 mmol) in THF (170 mL) were added acetic anhydride (28 mL, 277 mmol), 4-Dimethyl amino pyridine (3.38 g, 27.75 mmol) at 0° C. Then the reaction mixture was stirred at room temperature for 16 h. After completion, the reaction mixture was diluted with cold water, extracted with ethyl acetate and the organic layer was dried over anhydrous sodium sulfate, filtered, concentrated under reduced pressure. The crude residue obtained was purified by C18 reverse phase column chromatography using acetonitrile and water (+0.1% TFA) as eluents to afford the desired product (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-(difluoromethyl)phenyl)acetamido)propane-1,2-diyl diacetate (6) as off white solid, Yield: 11 g, 42%; LCMS (ESI+) m/z 781.6 [M+H]⁺.

### Synthesis of (1R,2R)-1-((2R,3R,4S,6R)-4-acetoxy-3-(2-acetoxyacetamido)-6-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-6-(methoxycarbonyl)tetrahydro-2H-pyran-2-yl)-3-(2-(4-(difluoromethyl)phenyl)acetamido)propane-1,2-diyl diacetate (8)

To a stirred solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-(difluoromethyl)phenyl)acetamido)propane-1,2-diyl diacetate (6) (2 g, 2.56 mmol) and tert-butyl (14-((2-((6-hydroxyhexyl)amino)-3,4-dioxocyclobut-1-en-1-yl)amino)-3,6,9,12-tetraoxatetradecyl)carbamate (7) (1.43 g, 3.07 mmol), Silver (1+) trifluoro methane sulfonate (AgOTf) (1.64 g, 6.40 mmol) in anhydrous dichloromethane (12 mL) and anhydrous acetonitrile (24 mL) was added activated 4 Å powdered molecular sieves (5 g). Then the reaction mixture was stirred at room temperature for 1 h under nitrogen atmosphere. The solution was cooled to −78° C., iodine monobromide (794 mg, 3.84 mmol) dissolved in dichloromethane (4 mL) was added dropwise, and the resulting mixture was stirred at same temperature for 2 h. After completion, the reaction mixture was quenched by addition of triethylamine (5 mL) at the same temperature and warmed to room temperature. The reaction mixture was filtered, the filtrate was washed with aqueous sodium thiosulfate solution, dried over sodium sulfate, filtered, and concentrated under reduced pressure. The crude residue obtained was purified by C18 reverse phase column chromatography using water: acetonitrile (+0.05% TFA) as eluents to afford the desired product (1R,2R)-1-((2R,3R,4S,6R)-4-acetoxy-3-(2-acetoxyacetamido)-6-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-6-(methoxycarbonyl)tetrahydro-2H-pyran-2-yl)-3-(2-(4-(difluoromethyl)phenyl)acetamido)propane-1,2-diyl diacetate (8) as a pale yellow solid, Yield: 1.5 g, 49%; LCMS (ESI+) m/z 1089.0 [M+H]⁺.

### Synthesis of (2R,4S,5R,6R)-6-((1R,2R)-3-(2-(4-(difluoromethyl)phenyl)acetamido)-1,2-dihydroxypropyl)-2-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-4-hydroxy-5-(2-hydroxyacetamido)tetrahydro-2H-pyran-2-carboxylic acid (9)

To a stirred solution of (1R,2R)-1-((2R,3R,4S,6R)-4-acetoxy-3-(2-acetoxyacetamido)-6-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-6-(methoxycarbonyl)tetrahydro-2H-pyran-2-yl)-3-(2-(4-(difluoromethyl)phenyl)acetamido)propane-1,2-diyl diacetate (8) (1.5 g, 1.26 mmol) in methanol (14 mL) and water (4 mL) was added NaOMe (25% in MeOH) (0.57 mL, 2.52 mmol) dropwise at room temperature. The resulting reaction mixture was stirred at room temperature for 16 h. After completion, the reaction mixture was treated with acidic (Dowex 50, H⁺) to maintain pH˜6 and the suspension was filtered. The filtrate was concentrated under reduced pressure and the crude was purified by C18 reverse phase column chromatography using water: acetonitrile (+0.05% TFA) as eluents to afford the desired product (2R,4S,5R,6R)-6-((1R,2R)-3-(2-(4-(difluoromethyl)phenyl)acetamido)-1,2-dihydroxypropyl)-2-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-4-hydroxy-5-(2-hydroxyacetamido)tetrahydro-2H-pyran-2-carboxylic acid (9) as an off white solid, Yield: 800 mg, 62%; LCMS (ESI+) m/z 1006.9 [M+H]⁺.

### Synthesis of (2R,4S,5R,6R)-2-((6-((2-((14-amino-3,6,9,12-tetraoxatetradecyl)amino)-3,4-dioxocyclobut-1-en-1-yl)amino)hexyl)oxy)-6-((1R,2R)-3-(2-(4-(difluoromethyl)phenyl)acetamido)-1,2-dihydroxypropyl)-4-hydroxy-5-(2-hydroxyacetamido)tetrahydro-2H-pyran-2-carboxylic acid (10)

To a stirred solution of (2R,4S,5R,6R)-6-((1R,2R)-3-(2-(4-(difluoromethyl)phenyl)acetamido)-1,2-dihydroxypropyl)-2-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-4-hydroxy-5-(2-hydroxyacetamido)tetrahydro-2H-pyran-2-carboxylic acid (9) (800 mg, 0.79 mmol) in anhydrous dichloromethane (8 mL) was added trifluoro acetic acid (2.34 mL) dropwise at 0° C. and the resulting reaction mixture was stirred at room temperature for 3 h. After completion, the solvent was concentrated to get crude which was purified by prep-HPLC (15-80% acetonitrile in water with +0.05% TFA). Pure fraction was lyophilized to afford the desired product (2R,4S,5R,6R)-2-((6-((2-((14-amino-3,6,9,12-tetraoxatetradecyl)amino)-3,4-dioxocyclobut-1-en-1-yl)amino)hexyl)oxy)-6-((1R,2R)-3-(2-(4-(difluoromethyl)phenyl)acetamido)-1,2-dihydroxypropyl)-4-hydroxy-5-(2-hydroxyacetamido)tetrahydro-2H-pyran-2-carboxylic acid (10) as a yellow solid, Yield: 320 mg, 44%; LCMS (ESI+) m/z 906.8 [M+H]⁺.

### Synthesis of (2R,4S,5R,6R)-6-((1R,2R)-3-(2-(4-(difluoromethyl)phenyl)acetamido)-1,2-dihydroxypropyl)-2-((6-((3,4-dioxo-2-((2-oxo-6,9,12,15-tetraoxa-3-azaheptadecan-17-yl)amino)cyclobut-1-en-1-yl)amino)hexyl)oxy)-4-hydroxy-5-(2-hydroxyacetamido)tetrahydro-2H-pyran-2-carboxylic acid (258)

To a stirred solution of (2R,4S,5R,6R)-2-((6-((2-((14-amino-3,6,9,12-tetraoxatetradecyl)amino)-3,4-dioxocyclobut-1-en-1-yl)amino)hexyl)oxy)-6-((1R,2R)-3-(2-(4-(difluoromethyl)phenyl)acetamido)-1,2-dihydroxypropyl)-4-hydroxy-5-(2-hydroxyacetamido)tetrahydro-2H-pyran-2-carboxylic acid (10) (70 mg, 0.08 mmol) in DMF (0.7 mL) was added 2,5-dioxopyrrolidin-1-yl acetate (11) (12 mg, 0.08 mmol) at 0° C. The resulting reaction mixture was stirred at 0° C. for 1 h. After completion, the reaction mixture was purified by prep-HPLC purification using (15-80% acetonitrile in water with +0.05% TFA) to afford the desired product (2R,4S,5R,6R)-6-((1R,2R)-3-(2-(4-(difluoromethyl)phenyl)acetamido)-1,2-dihydroxypropyl)-2-((6-((3,4-dioxo-2-((2-oxo-6,9,12,15-tetraoxa-3-azaheptadecan-17-yl)amino)cyclobut-1-en-1-yl)amino)hexyl)oxy)-4-hydroxy-5-(2-hydroxyacetamido)tetrahydro-2H-pyran-2-carboxylic acid (258) as white solid, Yield: 30 mg, 41%; LCMS (ESI+) m/z 948.9 [M+H]⁺. ¹H NMR (400 MHz, MeOD) δ 7.47 (dd, J=22.0, 8.0 Hz, 4H), 6.75 (t, J=56 Hz, 1H), 4.03 (s, 2H), 3.91-3.70 (m, 8H), 3.66-3.64 (m, 18H), 3.63-3.23 (m, 6H), 2.76-2.71 (m, 1H), 1.96 (s, 3H), 1.72-1.38 (m, 10H).

### (2R,4S,5R,6R)-6-((1R,2R)-3-(2-(4-(difluoromethyl)phenyl)acetamido)-1,2-dihydroxypropyl)-2-((6-((2-((18-(2,5-dioxo-2,5-dihydro-1H-pyrrol-1-yl)-16-oxo-3,6,9,12-tetraoxa-15-azaoctadecyl)amino)-3,4-dioxocyclobut-1-en-1-yl)amino)hexyl)oxy)-4-hydroxy-5-(2-hydroxyacetamido)tetrahydro-2H-pyran-2-carboxylic acid (Cpd. ST-57b)

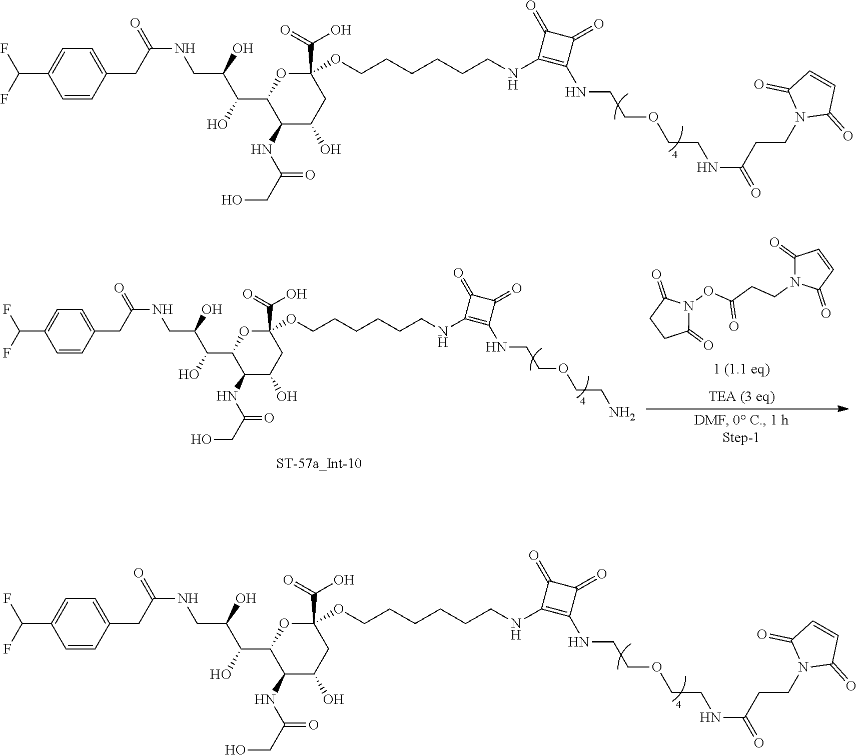

### Synthesis of (2R,4S,5R,6R)-6-((1R,2R)-3-(2-(4-(difluoromethyl)phenyl)acetamido)-1,2-dihydroxypropyl)-2-((6-((2-((18-(2,5-dioxo-2,5-dihydro-1H-pyrrol-1-yl)-16-oxo-3,6,9,12-tetraoxa-15-azaoctadecyl)amino)-3,4-dioxocyclobut-1-en-1-yl)amino)hexyl)oxy)-4-hydroxy-5-(2-hydroxyacetamido)tetrahydro-2H-pyran-2-carboxylic acid (258M)

To a stirred solution of (2R,4S,5R,6R)-2-((6-((2-((14-amino-3,6,9,12-tetraoxatetradecyl)amino)-3,4-dioxocyclobut-1-en-1-yl)amino)hexyl)oxy)-6-((1R,2R)-3-(2-(4-(difluoromethyl)phenyl)acetamido)-1,2-dihydroxypropyl)-4-hydroxy-5-(2-hydroxyacetamido)tetrahydro-2H-pyran-2-carboxylic acid (258_Int-10) (150 mg, 0.165 mmol) in DMF (1.5 mL) were added 2,5-dioxopyrrolidin-1-yl 3-(2,5-dioxo-2,5-dihydro-1H-pyrrol-1-yl)propanoate (1) (48 mg, 0.182 mmol) and triethylamine (0.05 mL, 0.495 mmol) at 0° C. and the resulting reaction mixture was stirred at the same temperature for 1 h. After completion, the reaction mixture was purified by prep-HPLC purification using acetonitrile and water (+0.1% TFA) to afford the desired product (2R,4S,5R,6R)-6-((1R,2R)-3-(2-(4-(difluoromethyl)phenyl)acetamido)-1,2-dihydroxypropyl)-2-((6-((2-((18-(2,5-dioxo-2,5-dihydro-1H-pyrrol-1-yl)-16-oxo-3,6,9,12-tetraoxa-15-azaoctadecyl)amino)-3,4-dioxocyclobut-1-en-1-yl)amino)hexyl)oxy)-4-hydroxy-5-(2-hydroxyacetamido)tetrahydro-2H-pyran-2-carboxylic acid (258M) as a white solid, Yield: 41 mg, 23%; LCMS (ESI+) m/z 1057.9 [M+H]⁺. ¹H NMR (400 MHz, MeOD) δ 7.48 (dd, J=20, 8 Hz, 4H), 6.84 (s, 2H), 6.76 (t, J=56 Hz, 1H), 4.05 (s, 2H), 3.92-3.70 (m, 10H), 3.67-3.40 (m, 23H), 3.34-3.26 (m, 1H), 2.80-2.73 (m, 1H), 2.50 (t, J=8 Hz, 2H), 1.76-1.32 (m, 10H).

### (2R,4S,5R,6R)-6-((1R,2R)-3-(2-(4-(difluoromethyl)phenyl)acetamido)-1,2-dihydroxypropyl)-2-((6-((2-((16,32-dioxo-36-((3aR,4R,6aS)-2-oxohexahydro-1H-thieno[3,4-d]imidazol-4-yl)-3,6,9,12,19,22,25,28-octaoxa-15,31-diazahexatriacontyl)amino)-3,4-dioxocyclobut-1-en-1-yl)amino)hexyl)oxy)-4-hydroxy-5-(2-hydroxyacetamido)tetrahydro-2H-pyran-2-carboxylic acid (Cpd. 258B)

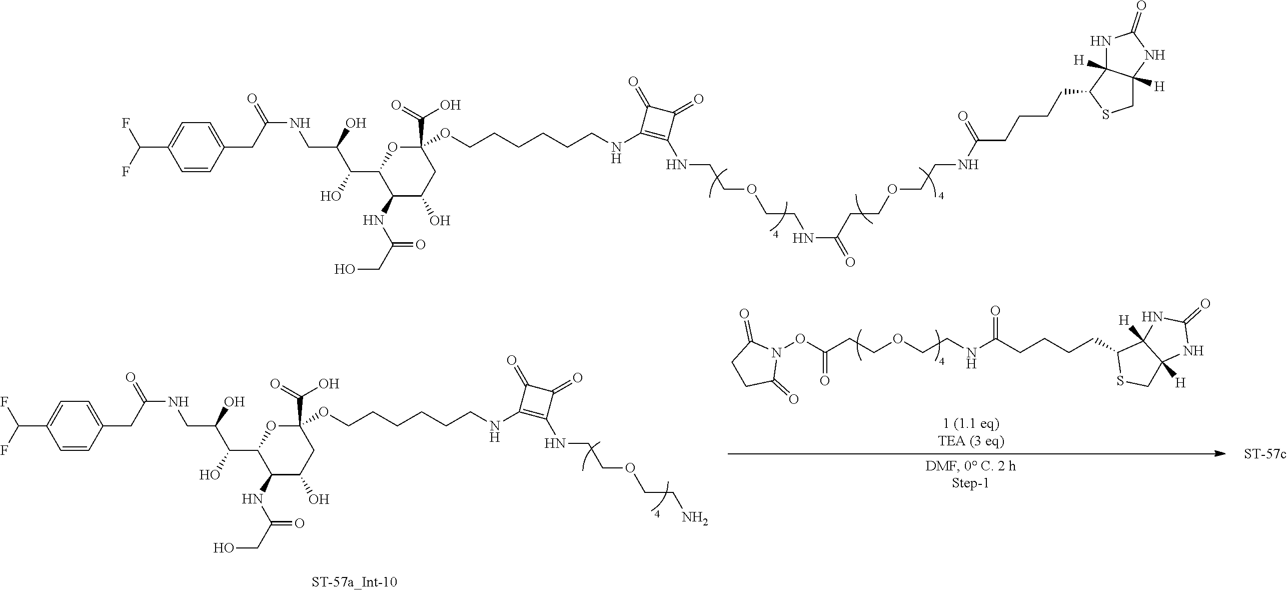

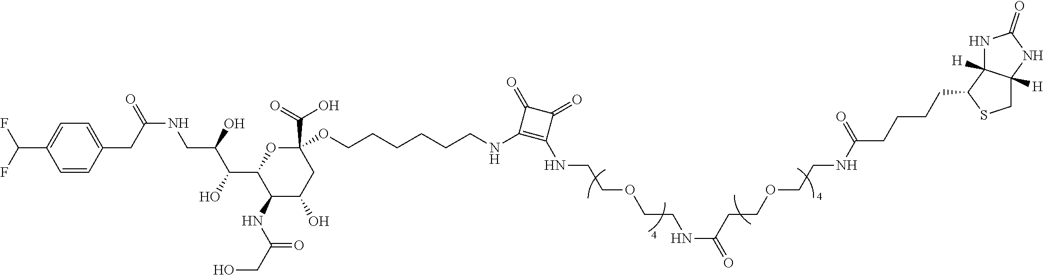

### Synthesis of (2R,4S,5R,6R)-6-((1R,2R)-3-(2-(4-(difluoromethyl)phenyl)acetamido)-1,2-dihydroxypropyl)-2-((6-((2-((16,32-dioxo-36-((3aR,4R,6aS)-2-oxohexahydro-1H-thieno[3,4-d]imidazol-4-yl)-3,6,9,12,19,22,25,28-octaoxa-15,31-diazahexatriacontyl)amino)-3,4-dioxocyclobut-1-en-1-yl)amino)hexyl)oxy)-4-hydroxy-5-(2-hydroxyacetamido)tetrahydro-2H-pyran-2-carboxylic acid (258B)

To a stirred solution of (2R,4S,5R,6R)-2-((6-((2-((14-amino-3,6,9,12-tetraoxatetradecyl)amino)-3,4-dioxocyclobut-1-en-1-yl)amino)hexyl)oxy)-6-((1R,2R)-3-(2-(4-(difluoromethyl)phenyl)acetamido)-1,2-dihydroxypropyl)-4-hydroxy-5-(2-hydroxyacetamido)tetrahydro-2H-pyran-2-carboxylic acid (258_Int-10) (50 mg, 0.06 mmol) in DMF (0.5 mL) were added 2,5-dioxopyrrolidin-1-yl 17-oxo-21-((3aR,4R,6aS)-2-oxohexahydro-1H-thieno[3,4-d]imidazol-4-yl)-4,7,10,13-tetraoxa-16-azahenicosanoate (1) (35 mg, 0.06 mmol) and triethylamine (0.02 mL, 0.17 mmol) at 0° C. The resulting reaction mixture was stirred at the same temperature for 1 h. After completion, the reaction mixture was purified by prep-HPLC purification using acetonitrile and water (+0.1% TFA) as eluents to afford the desired product (2R,4S,5R,6R)-6-((1R,2R)-3-(2-(4-(difluoromethyl)phenyl)acetamido)-1,2-dihydroxypropyl)-2-((6-((2-((16,32-dioxo-36-((3aR,4R,6aS)-2-oxohexahydro-1H-thieno[3,4-d]imidazol-4-yl)-3,6,9,12,19,22,25,28-octaoxa-15,31-diazahexatriacontyl)amino)-3,4-dioxocyclobut-1-en-1-yl)amino)hexyl)oxy)-4-hydroxy-5-(2-hydroxyacetamido)tetrahydro-2H-pyran-2-carboxylic acid (258B) as white solid, Yield: 30 mg, 40%; LCMS (ESI+) m/z 1380.6 [M+H]⁺. ¹H NMR (400 MHz, MeOD) δ 7.47 (dd, J=21.6, 8 Hz, 4H), 6.84 (s, 2H), 6.75 (t, J=56 Hz, 1H), 4.53-4.50 (m, 1H), 4.38-4.30 (m, 1H), 4.03 (s, 2H), 3.83-3.70 (m, 9H), 3.66-3.61 (m, 28H), 3.57-3.22 (m, 15H), 2.98-2.92 (m, 1H), 2.75-2.69 (m, 2H), 2.48 (t, J=6 Hz, 2H), 2.26-2.22 (m, 2H), 1.70-1.39 (m, 16H).

### (2R,4S,5R,6R)-6-((1R,2R)-3-(2-(4-(1H-tetrazol-5-yl) phenyl)acetamido)-1,2-dihydroxypropyl)-2-((2,18-dioxo-6,9,12,15-tetraoxa-3,19-diazapentacosan-25-yl)oxy)-4-hydroxy-5-(2-hydroxyacetamido)tetrahydro-2H-pyran-2-carboxylic acid (Cpd. 235)

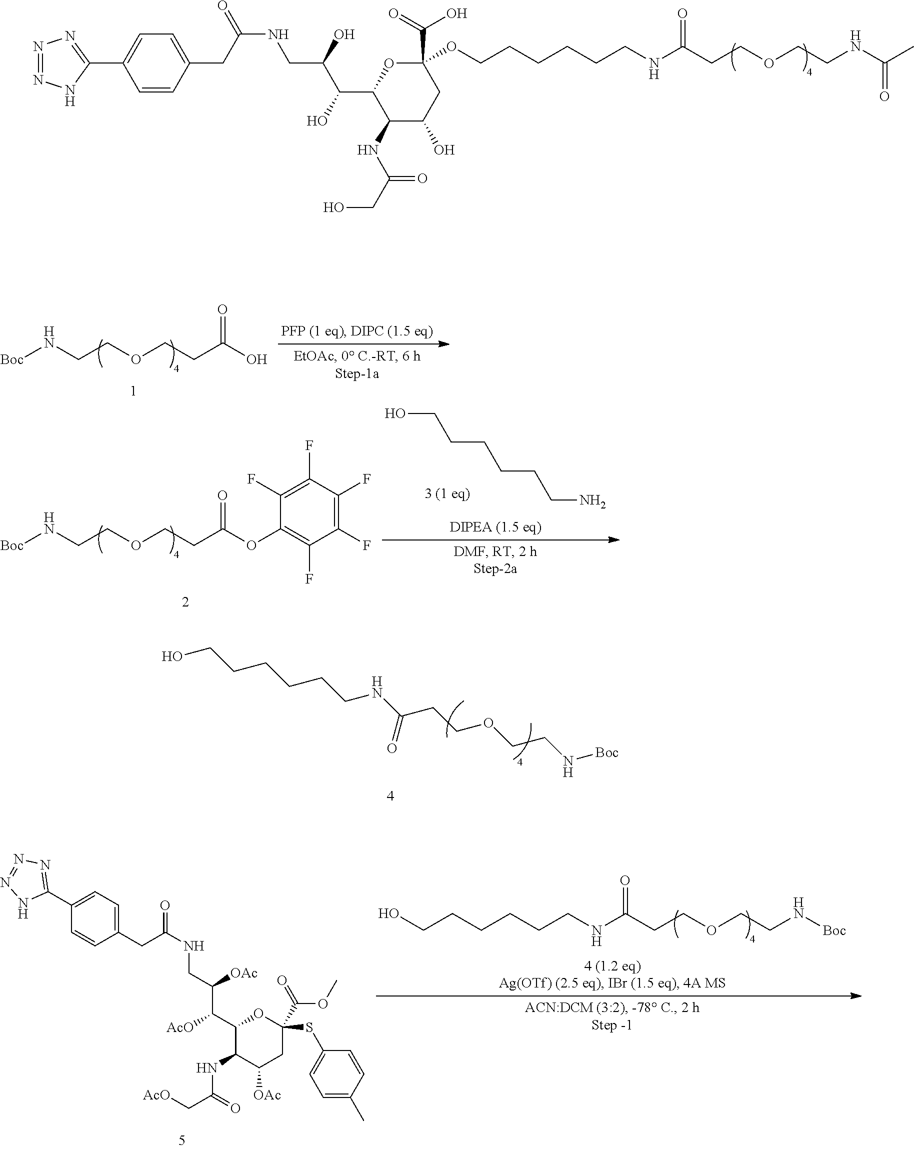

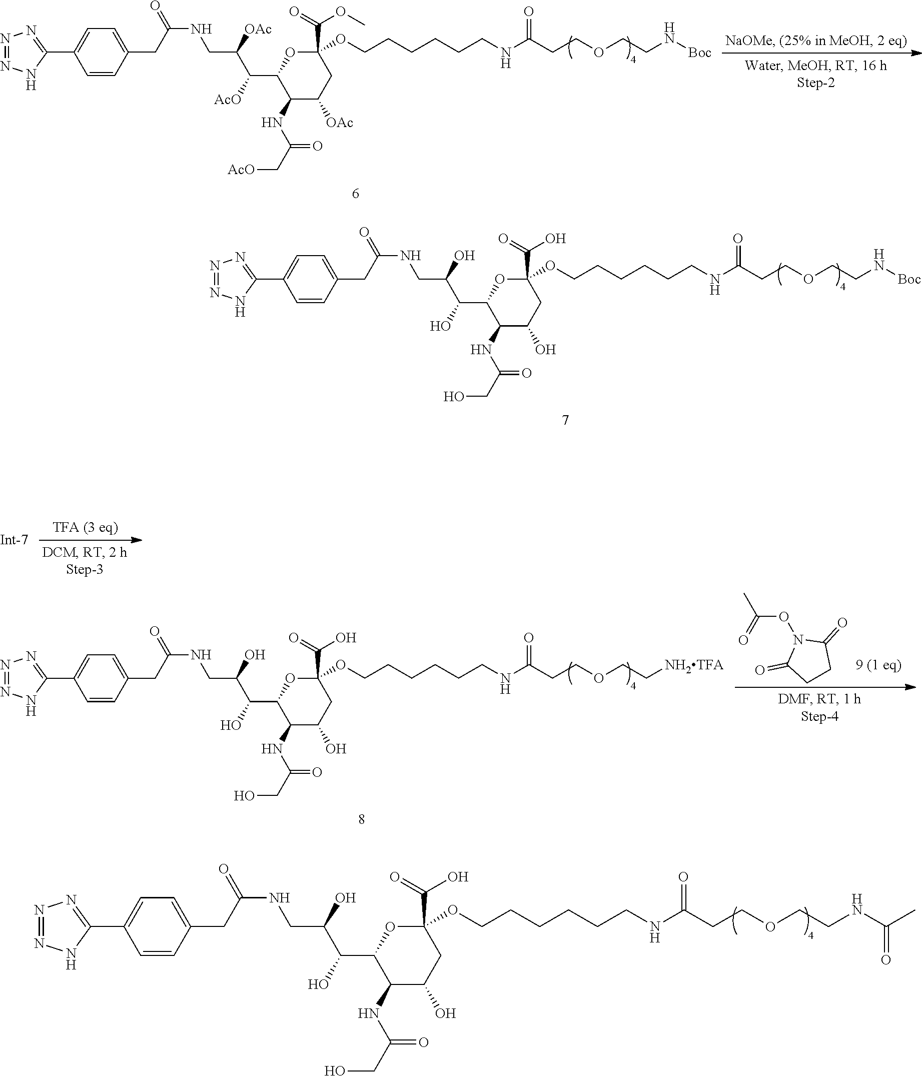

### Synthesis ofperfluorophenyl 2,2-dimethyl-4-oxo-3,8,11,14,17-pentaoxa-5-azaicosan-20-oate (2)

To a stirred solution of 2,2-dimethyl-4-oxo-3,8,11,14,17-pentaoxa-5-azaicosan-20-oic acid (1) (5 g, 13.6 mmol) and penta-fluoro phenol (2.5 g, 13.6 mmol) in ethyl acetate (50 mL) was added di-iso-propyl carbodimide (2.58 g, 20.4 mmol) dropwise at 0° C. and the reaction mixture was stirred at room temperature for 6 h. After completion, the reaction mixture was filtered, filtrate was concentrated, the crude residue was purified by Silica gel column chromatography using ethyl acetate and hexane as eluents to afford perfluorophenyl 2,2-dimethyl-4-oxo-3,8,11,14,17-pentaoxa-5-azaicosan-20-oate (2) as off-white solid, Yield: 5 g, 69.44%. LCMS (ESI±) m/z 533.65 [M+H]⁺.

### Synthesis of tert-butyl (22-hydroxy-15-oxo-3,6,9,12-tetraoxa-16-azadocosyl)carbamate (4)

To a stirred solution of perfluorophenyl 2,2-dimethyl-4-oxo-3,8,11,14,17-pentaoxa-5-azaicosan-20-oate (2) (4 g, 7.52 mmol) and 6-aminohexan-1-ol (3) (0.88 g, 7.52 mmol) in N,N-dimethylformamide (10 mL) was added di-isopropyl ethylamine (2.08 ml, 11.29 mmol) at 0° C. The resulting reaction mixture was stirred at room temperature for 2 h. After completion, the reaction mixture was concentrated and purified by column chromatography using ethyl acetate and hexane as eluents to afford tert-butyl (22-hydroxy-15-oxo-3,6,9,12-tetraoxa-16-azadocosyl)carbamate (4) as white solid, Yield: 2.4 g, 70.58%. LCMS (ESI±) m/z 465.5 [M+H]⁺.

### Synthesis of (1R,2R)-3-(2-(4-(1H-tetrazol-5-yl)phenyl)acetamido)-1-((2R,3R,4S,6R)-4-acetoxy-3-(2-acetoxyacetamido)-6-((2,2-dimethyl-4,20-dioxo-3,8,11,14,17-pentaoxa-5,21-diazaheptacosan-27-yl)oxy)-6-(methoxycarbonyl)tetrahydro-2H-pyran-2-yl)propane-1,2-diyl diacetate (6)

To a stirred solution of (1R,2R)-3-(2-(4-(1H-tetrazol-5-yl)phenyl)acetamido)-1-((2R,3R,4S,6R)-4-acetoxy-3-(2-acetoxyacetamido)-6-(methoxycarbonyl)-6-(p-tolylthio)tetrahydro-2H-pyran-2-yl)propane-1,2-diyl diacetate (5) (2 g, 2.5 mmol), tert-butyl (22-hydroxy-15-oxo-3,6,9,12-tetraoxa-16-azadocosyl)carbamate (4) (1.39 g, 3 mmol) and Silver (1+) trifluoro methane sulfonate (AgOTf) (1.61 g, 6.26 mmol) in anhydrous dichloromethane (12 mL) and anhydrous acetonitrile (24 mL) was added activated 4 Å powdered molecular sieves (5 g) and the reaction mixture was stirred at room temperature for 1 h under nitrogen. Then the solution was cooled to −78° C., iodine monobromide (0.78 g, 3.75 mmol) dissolved in dichloromethane (4 mL) was added dropwise, and the resulting mixture was stirred at same temperature for 2 h. After completion, the reaction mixture was quenched by addition of triethylamine (5 mL) and allowed to stir at room temperature for 1 h. The reaction mixture was filtered, the filtrate was washed with aqueous sodium thiosulfate solution, dried over sodium sulfate, filtered, and concentrated under reduced pressure. The crude residue obtained was purified by C18 reverse phase column chromatography using water and acetonitrile (+0.05% TFA) as eluents to afford the desired product (1R,2R)-3-(2-(4-(1H-tetrazol-5-yl)phenyl)acetamido)-1-((2R,3R,4S,6R)-4-acetoxy-3-(2-acetoxyacetamido)-6-((2,2-dimethyl-4,20-dioxo-3,8,11,14,17-pentaoxa-5,21-diazaheptacosan-27-yl)oxy)-6-(methoxycarbonyl)tetrahydro-2H-pyran-2-yl)propane-1,2-diyl diacetate (6) as off-white solid, Yield: 650 mg, 23%; LCMS (ESI±) m/z 1138.1 [M−H]⁻.

### Synthesis of (2R,4S,5R,6R)-6-((1R,2R)-3-(2-(4-(1H-tetrazol-5-yl)phenyl)acetamido)-1,2-dihydroxypropyl)-2-((2,2-dimethyl-4,20-dioxo-3,8,11,14,17-pentaoxa-5,21-diazaheptacosan-27-yl)oxy)-4-hydroxy-5-(2-hydroxyacetamido)tetrahydro-2H-pyran-2-carboxylic acid (7)

To a stirred solution of (1R,2R)-3-(2-(4-(1H-tetrazol-5-yl)phenyl)acetamido)-1-((2R,3R,4S,6R)-4-acetoxy-3-(2-acetoxyacetamido)-6-((2,2-dimethyl-4,20-dioxo-3,8,11,14,17-pentaoxa-5,21-diazaheptacosan-27-yl)oxy)-6-(methoxycarbonyl)tetrahydro-2H-pyran-2-yl)propane-1,2-diyl diacetate (6) (650 mg, 0.57 mmol) in methanol (10 mL) and water (3 mL) was added NaOMe (25% in MeOH) (0.26 mL, 1.14 mmol) dropwise at 0° C. The resulting reaction mixture was stirred at room temperature for 16 h. After completion, the reaction mixture was treated with acidic resin (Dowex 50, H⁺) to maintain pH˜6 and the suspension was filtered. The filtrate was concentrated under reduced pressure and the crude residue obtained was purified by C18 reverse phase column chromatography using water and acetonitrile (+0.05% TFA) as eluents to afford the desired product (2R,4S,5R,6R)-6-((1R,2R)-3-(2-(4-(1H-tetrazol-5-yl)phenyl)acetamido)-1,2-dihydroxypropyl)-2-((2,2-dimethyl-4,20-dioxo-3,8,11,14,17-pentaoxa-5,21-diazaheptacosan-27-yl)oxy)-4-hydroxy-5-(2-hydroxyacetamido)tetrahydro-2H-pyran-2-carboxylic acid (7) as off-white solid, Yield: 350 mg, 64%; LCMS (ESI+) m/z 957.7 [M+H]⁺.

### Synthesis of (2R,4S,5R,6R)-6-((1R,2R)-3-(2-(4-(1H-tetrazol-5-yl) phenyl) acetamido)-1,2-dihydroxypropyl)-2-((1-amino-15-oxo-3,6,9,12-tetraoxa-16-azad° C. osan-22-yl)oxy)-4-hydroxy-5-(2-hydroxyacetamido)tetrahydro-2H-pyran-2-carboxylic acid (8)

To a stirred solution of (2R,4S,5R,6R)-6-((1R,2R)-3-(2-(4-(1H-tetrazol-5-yl)phenyl)acetamido)-1,2-dihydroxypropyl)-2-((2,2-dimethyl-4,20-dioxo-3,8,11,14,17-pentaoxa-5,21-diazaheptacosan-27-yl)oxy)-4-hydroxy-5-(2-hydroxyacetamido)tetrahydro-2H-pyran-2-carboxylic acid (7) (350 mg, 0.37 mmol) in anhydrous dichloromethane (4 mL) was added trifluoro acetic acid (0.03 mL) dropwise at 0° C. and the resulting reaction mixture was stirred at room temperature for 3 h. After completion, the solvent was concentrated and the crude residue obtained was purified by prep-HPLC (15-80% acetonitrile in water, +0.05% TFA) to afford the desired product (2R,4S,5R,6R)-6-((1R,2R)-3-(2-(4-(1H-tetrazol-5-yl)phenyl)acetamido)-1,2-dihydroxypropyl)-2-((1-amino-15-oxo-3,6,9,12-tetraoxa-16-azad° C. osan-22-yl)oxy)-4-hydroxy-5-(2-hydroxyacetamido)tetrahydro-2H-pyran-2-carboxylic acid (8) as off-white solid, Yield: 260 mg, 83%. LCMS (ESI±) m/z 857.8 [M+H]⁺.

### Synthesis of (2R,4S,5R,6R)-6-((1R,2R)-3-(2-(4-(1H-tetrazol-5-yl) phenyl) acetamido)-1,2-dihydroxypropyl)-2-((2,18-dioxo-6,9,12,15-tetraoxa-3,19-diazapentacosan-25-yl) oxy)-4-hydroxy-5-(2-hydroxyacetamido) tetrahydro-2H-pyran-2-carboxylic acid (235)

To a stirred solution of (2R,4S,5R,6R)-6-((1R,2R)-3-(2-(4-(1H-tetrazol-5-yl)phenyl)acetamido)-1,2-dihydroxypropyl)-2-((1-amino-15-oxo-3,6,9,12-tetraoxa-16-azad° C. osan-22-yl)oxy)-4-hydroxy-5-(2-hydroxyacetamido)tetrahydro-2H-pyran-2-carboxylic acid (8) (100 mg, 0.12 mmol) in DMF (1 mL) were added 2,5-dioxopyrrolidin-1-yl acetate (9) (19 mg, 0.12 mmol) at 0° C. The resulting reaction mixture was stirred at room temperature for 1 h. After completion, the reaction mixture was concentrated and purified by prep-HPLC using acetonitrile and water (+0.05%, TFA) to afford the desired product (2R,4S,5R,6R)-6-((1R,2R)-3-(2-(4-(1H-tetrazol-5-yl)phenyl)acetamido)-1,2-dihydroxypropyl)-2-((2,18-dioxo-6,9,12,15-tetraoxa-3,19-diazapentacosan-25-yl)oxy)-4-hydroxy-5-(2-hydroxyacetamido)tetrahydro-2H-pyran-2-carboxylic acid (235) as a white solid, Yield: 37 mg, 35.3%. LCMS (ESI+) m/z 899.4 [M+H]⁺.

¹H NMR (400 MHz, MeOD) δ 7.98 (d, J=8.4 Hz, 2H), 7.55 (d, J=8.4 Hz, 2H), 4.02 (s, 2H), 3.92-3.62 (m, 22H), 3.55-3.53 (m, 2H), 3.41-3.26 (m, 5H), 3.19-3.15 (m, 2H), 2.71 (dd, J=16.0 Hz, 1H), 2.45-2.42 (m, 2H), 1.95 (s, 3H), 1.76-1.70 (m, 1H), 1.54-1.47 (m, 4H), 1.34-1.31 (m, 4H).

### (2R,4S,5R,6R)-6-((1R,2R)-3-(2-(4-(1H-tetrazol-5-yl)phenyl)acetamido)-1,2-dihydroxypropyl)-2-((1-(2,5-dioxo-2,5-dihydro-1H-pyrrol-1-yl)-3,19-dioxo-7,10,13,16-tetraoxa-4,20-diazahexacosan-26-yl)oxy)-4-hydroxy-5-(2-hydroxyacetamido)tetrahydro-2H-pyran-2-carboxylic acid (Cpd. 235M)

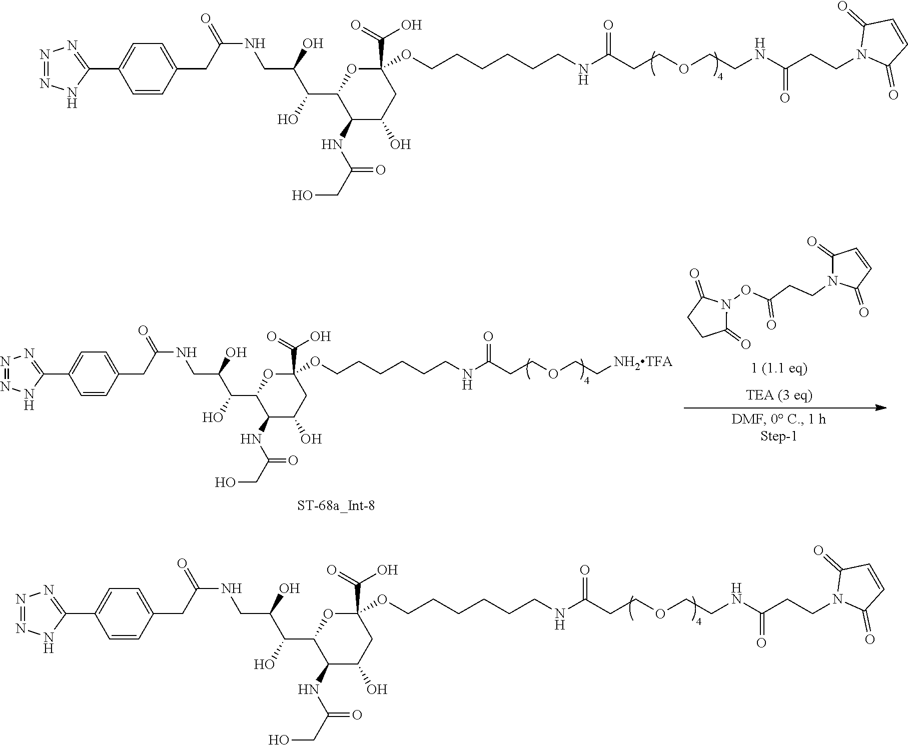

### Synthesis of (2R,4S,5R,6R)-6-((1R,2R)-3-(2-(4-(1H-tetrazol-5-yl)phenyl)acetamido)-1,2-dihydroxypropyl)-2-((1-(2,5-dioxo-2,5-dihydro-1H-pyrrol-1-yl)-3,19-dioxo-7,10,13,16-tetraoxa-4,20-diazahexacosan-26-yl)oxy)-4-hydroxy-5-(2-hydroxyacetamido)tetrahydro-2H-pyran-2-carboxylic acid (235M)

To a stirred solution of (2R,4S,5R,6R)-6-((1R,2R)-3-(2-(4-(1H-tetrazol-5-yl)phenyl)acetamido)-1,2-dihydroxypropyl)-4-hydroxy-5-(2-hydroxyacetamido)-2-((1,1,1-trifluoro-2,18-dioxo-6,9,12,15-tetraoxa-314,19-diazapentacosan-25-yl)oxy)tetrahydro-2H-pyran-2-carboxylic acid (ST-68a_Int-8) (110 mg, 0.12 mmol) in DMF (1.1 mL) were added 2,5-dioxopyrrolidin-1-yl 3-(2,5-dioxo-2,5-dihydro-1H-pyrrol-1-yl)propanoate (1) (35 mg, 0.13 mmol) and triethylamine (0.05 mL, 0.36 mmol) at 0° C. The resulting reaction mixture was stirred at the same temperature for 1 h. After completion, the reaction mixture was purified by prep-HPLC to afford the desired product (2R,4S,5R,6R)-6-((1R,2R)-3-(2-(4-(1H-tetrazol-5-yl)phenyl)acetamido)-1,2-dihydroxypropyl)-2-((1-(2,5-dioxo-2,5-dihydro-1H-pyrrol-1-yl)-3,19-dioxo-7,10,13,16-tetraoxa-4,20-diazahexacosan-26-yl)oxy)-4-hydroxy-5-(2-hydroxyacetamido)tetrahydro-2H-pyran-2-carboxylic acid (235M) as a white solid, Yield: 5.50 mg, 12%; LCMS (ESI+) m/z 1008.6 [M+H]⁺. ¹H NMR (400 MHz, MeOD) δ 7.99 (d, J=8.4 Hz, 2H), 7.55 (d, J=8.4 Hz, 2H), 6.83 (s, 2H), 4.02 (s, 2H), 3.81-3.69 (m, 9H), 3.67-3.28 (m, 23H), 3.17 (t, J=6.8 Hz, 2H), 2.78-2.73 (m, 1H), 2.50-2.43 (m, 4H), 1.75-1.31 (m, 10H).

### (2R,4S,5R,6R)-6-((1R,2R)-3-(2-(4-(1H-tetrazol-5-yl)phenyl)acetamido)-1,2-dihydroxypropyl)-4-hydroxy-5-(2-hydroxyacetamido)-2-((5,21,37-trioxo-1-((3aR,4R,6aS)-2-oxohexahydro-1H-thieno[3,4-d]imidazol-4-yl)-9,12,15,18,25,28,31,34-° C. taoxa-6,22,38-triazatetratetracontan-44-yl)oxy)tetrahydro-2H-pyran-2-carboxylic acid (Cpd. 235B)

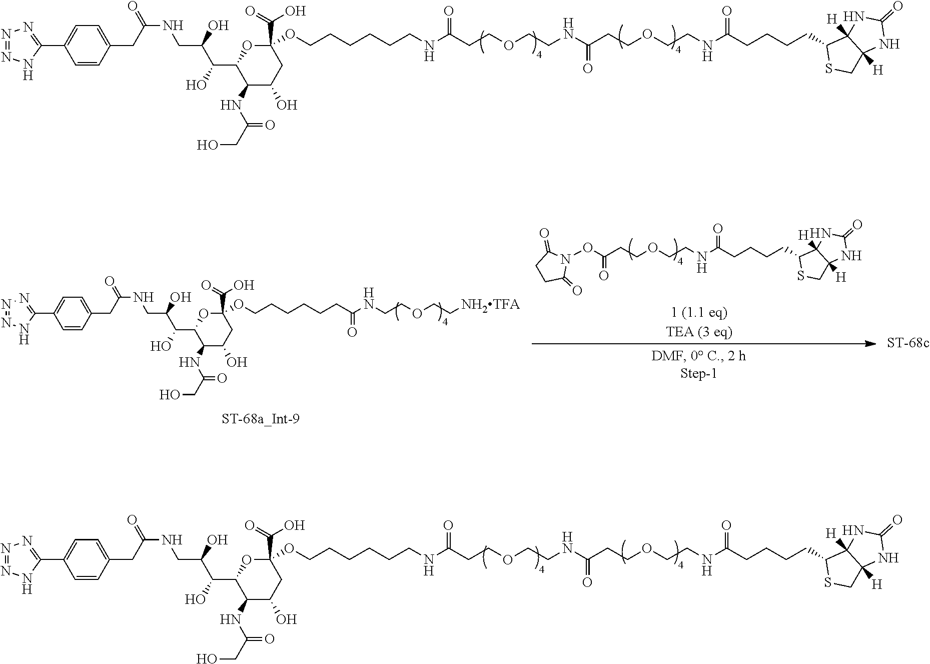

### Synthesis of (2R,4S,5R,6R)-6-((1R,2R)-3-(2-(4-(1H-tetrazol-5-yl)phenyl)acetamido)-1,2-dihydroxypropyl)-4-hydroxy-5-(2-hydroxyacetamido)-2-((5,21,37-trioxo-1-((3aR,4R,6aS)-2-oxohexahydro-1H-thieno[3,4-d]imidazol-4-yl)-9,12,15,18,25,28,31,34-° C. taoxa-6,22,38-triazatetratetracontan-44-yl)oxy)tetrahydro-2H-pyran-2-carboxylic acid (235B)

To a stirred solution (2R,4S,5R,6R)-6-((1R,2R)-3-(2-(4-(1H-tetrazol-5-yl)phenyl)acetamido)-1,2-dihydroxypropyl)-2-((1-amino-15-oxo-3,6,9,12-tetraoxa-16-azad° C. osan-22-yl)oxy)-4-hydroxy-5-(2-hydroxyacetamido)tetrahydro-2H-pyran-2-carboxylic acid (ST-68a_Int-9) (50 mg, 0.06 mmol) in N—N-Dimethylformamide (0.5 mL) were added 2,5-dioxopyrrolidin-1-yl 17-oxo-21-((3aR,4R,6aS)-2-oxohexahydro-1H-thieno[3,4-d]imidazol-4-yl)-4,7,10,13-tetraoxa-16-azahenicosanoate (1) (37.8 mg, 0.07 mmol) and triethyl amine (0.05 ml, 0.18 mmol) at 0° C. The resulting reaction mixture was stirred at the same temperature for 45 min. After completion, the reaction mixture was concentrated and purified by prep-HPLC using acetonitrile and water (+0.05% TFA) as eluents to afford desired product (2R,4S,5R,6R)-6-((1R,2R)-3-(2-(4-(1H-tetrazol-5-yl)phenyl)acetamido)-1,2-dihydroxypropyl)-4-hydroxy-5-(2-hydroxyacetamido)-2-((5,21,37-trioxo-1-((3aR,4R,6aS)-2-oxohexahydro-1H-thieno[3,4-d]imidazol-4-yl)-9,12,15,18,25,28,31,34-° C. taoxa-6,22,38-triazatetratetracontan-44-yl)oxy)tetrahydro-2H-pyran-2-carboxylic acid (235B) as white solid, Yield: 15 mg, 19.32%. LCMS (ESI±) m/z 1331.5 [M+H]⁺. ¹H NMR (400 MHz, MeOD): δ 7.99 (d, J=8.4 Hz, 2H), 7.56 (d, J=8 Hz, 2H), 4.50-4.31 (m, 2H), 4.03 (s, 2H), 3.83-3.68 (m, 9H), 3.65-3.54 (m, 31H), 3.41-3.17 (m, 10H), 2.98-2.92 (m, 1H), 2.74-2.70 (m, 2H), 2.49-2.44 (m, 4H), 2.23 (t, J=8 Hz, 2H), 1.74-1.35 (m, 16H).

### (2R,4S,5R,6R)-6-((1R,2R)-3-(4-chlorobenzamido)-1,2-dihydroxypropyl)-4-hydroxy-5-(2-hydroxyacetamido)-2-(oct-7-yn-1-yloxy) tetrahydro-2H-pyran-2-carboxylic acid (Cpd. 267)

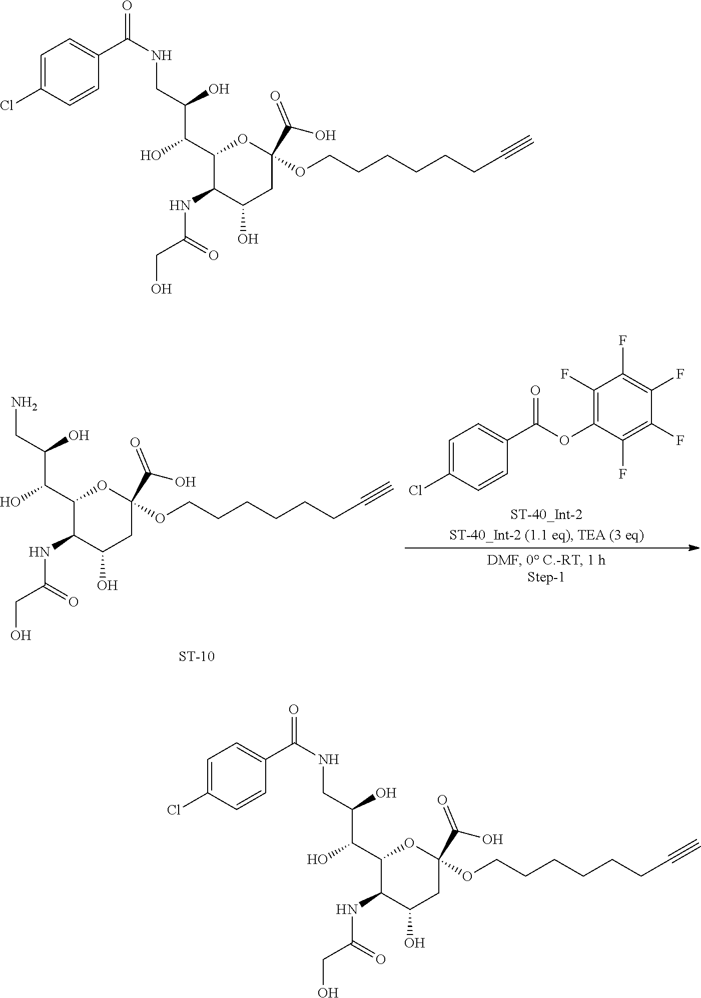

### Synthesis of (2R,4S,5R,6R)-6-((1R,2R)-3-(4-chlorobenzamido)-1,2-dihydroxypropyl)-4-hydroxy-5-(2-hydroxyacetamido)-2-(oct-7-yn-1-yloxy) tetrahydro-2H-pyran-2-carboxylic acid (Cpd. 267)

To a stirred solution of (2R,4S,5R,6R)-6-((1R,2R)-3-amino-1,2-dihydroxypropyl)-4-hydroxy-5-(2-hydroxyacetamido)-2-(oct-7-yn-1-yloxy)tetrahydro-2H-pyran-2-carboxylic acid (ST-10) (100 mg, 0.23 mmol) in N,N-dimethyl formamide (1 mL) were added perfluorophenyl 4-chlorobenzoate (251_Int-2) (82 mg, 0.25 mmol,) and triethylamine (70 mg, 0.69 mmol) at 0° C. The resulting reaction mixture was stirred at room temperature for 1 h. After completion, the reaction mixture was purified by prep-HPLC using acetonitrile and water (+0.05%, TFA) as eluents to afford the desired product (2R,4S,5R,6R)-6-((1R,2R)-3-(4-chlorobenzamido)-1,2-dihydroxypropyl)-4-hydroxy-5-(2-hydroxyacetamido)-2-(oct-7-yn-1-yloxy) tetrahydro-2H-pyran-2-carboxylic acid (267) as off white solid. Yield: 74 mg, 56%; LCMS (ESI±) m/z 571.25 [M+H]⁺. ¹H NMR (400 MHz, MeOD) δ 7.88-7.81 (m, 2H), 7.53-7.46 (m, 2H), 4.04 (s, 3H), 3.85 (td, J=8, 3.3 Hz, 3H), 3.79 (d, J=4.9 Hz, 2H), 3.58-3.41 (m, 3H), 2.80-2.69 (m, 1H), 2.17 (dt, J=7, 3.7 Hz, 3H), 1.77 (td, J=10, 4.4 Hz, 1H), 1.58 (q, J=6.8 Hz, 2H), 1.50 (q, J=7.1 Hz, 2H), 1.45-1.34 (m, 4H).

### (2R,4S,5R,6R)-6-((1R,2R)-3-(2-(4-(1H-tetrazol-5-yl) phenyl) acetamido)-1,2-dihydroxypropyl)-2-((2,19-dioxo-6,9,12,15-tetraoxa-3,18-diazapentacosan-25-yl) oxy)-4-hydroxy-5-(2-hydroxyacetamido) tetrahydro-2H-pyran-2-carboxylic acid (Cpd. 266)

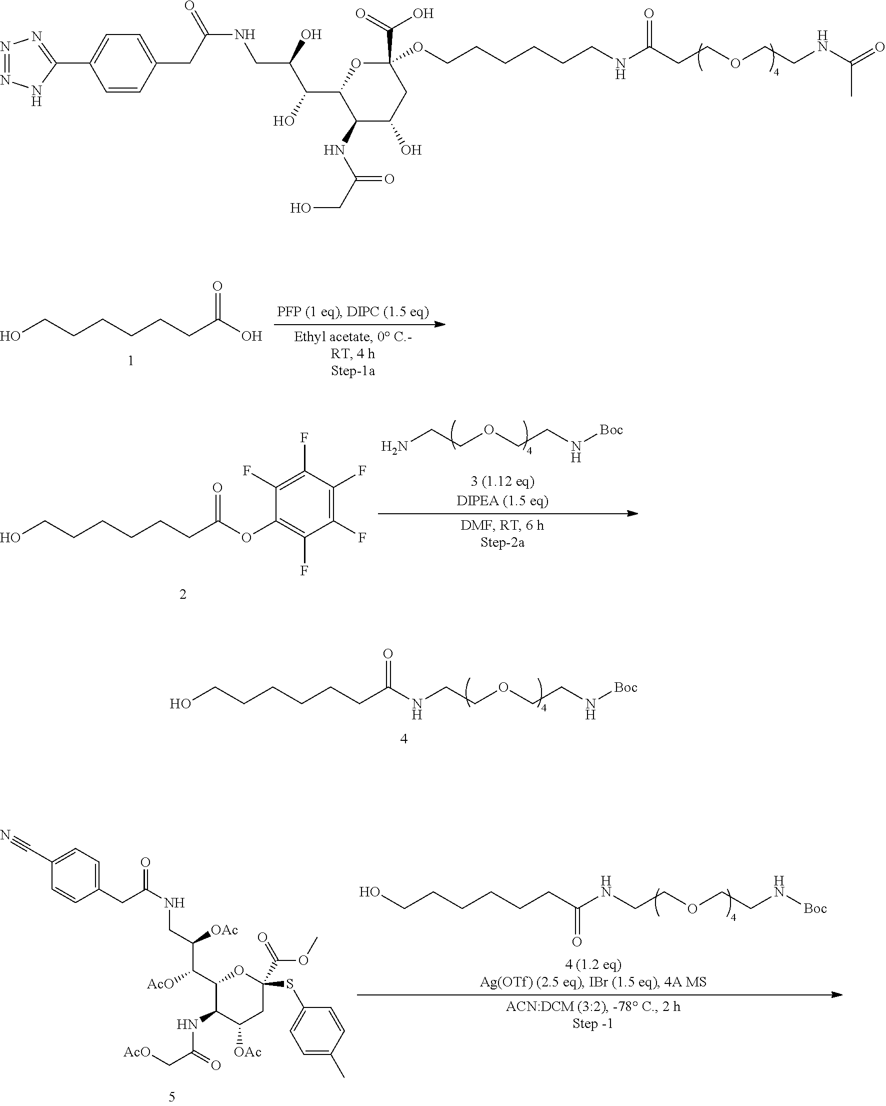

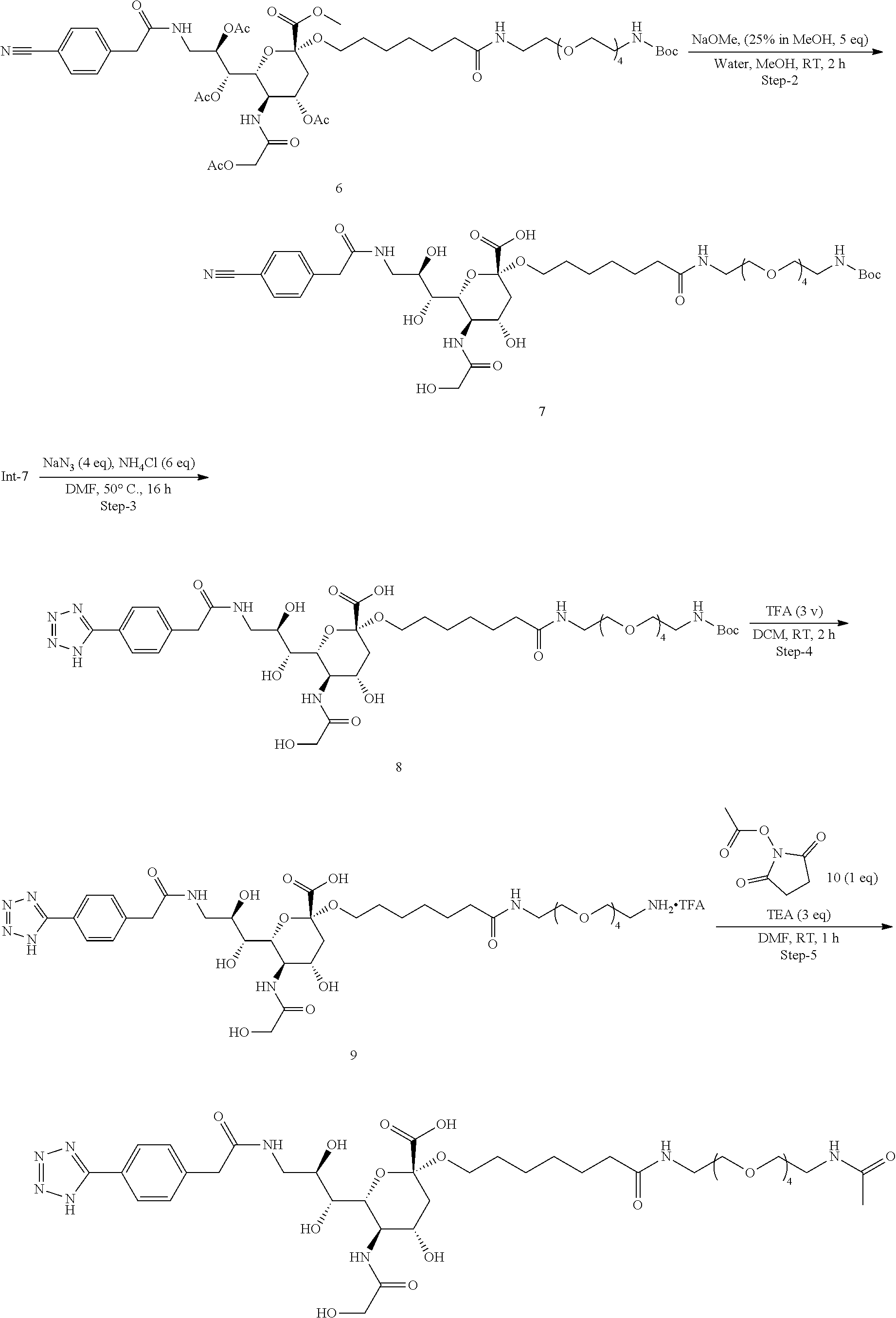

### Synthesis of perfluorophenyl 7-hydroxyheptanoate (2)

To a stirred solution of 7-hydroxyheptanoic acid (1) (5 g, 34 mmol) in EtOAc (50 mL) was added 2,3,4,5,6-pentafluorophenol (6.25 g, 34 mmol), followed by dropwise addition of N, N′-Di-isopropyl carbodiimide (6.43 g, 51 mmol) at 0° C. The resulting reaction mixture was stirred for 4 h at the same temperature. After completion, the reaction mixture was filtered, the residue was washed with ethyl acetate, and the filtrate was concentrated under reduced pressure to afford of the desired product perfluorophenyl 7-hydroxyheptanoate (2), which was used as such for the next step, Yield: 7 g, 65%.

### Synthesis of tert-butyl (22-hydroxy-16-oxo-3,6,9,12-tetraoxa-15-azadocosyl) carbamate (4)

To a stirred solution of perfluorophenyl 7-hydroxyheptanoate (2) (7 g, 22 mmol) in DMF (70 mL) were added tert-butyl (14-amino-3,6,9,12-tetraoxatetradecyl)carbamate (4) (7.5 g, 22 mmol), DIPEA (6 mL, 33 mmol) at 0° C. and the reaction mixture was stirred at the same temperature for 2 h. After completion, the reaction mixture was purified by C18 reverse phase column chromatography using water and acetonitrile (+0.05% TFA) as eluents to afford the desired product tert-butyl (22-hydroxy-16-oxo-3,6,9,12-tetraoxa-15-azadocosyl) carbamate (4) as off-white solid. Yield: 7 g, 67%; LCMS (ESI+) m/z 465.5 [M+H]⁺.

### Synthesis of (1R,2R)-1-((2R,3R,4S,6R)-4-acetoxy-3-(2-acetoxyacetamido)-6-((2,2-dimethyl-4,21-dioxo-3,8,11,14,17-pentaoxa-5,20-diazaheptacosan-27-yl)oxy)-6-(methoxycarbonyl)tetrahydro-2H-pyran-2-yl)-3-(2-(4-cyanophenyl)acetamido)propane-1,2-diyl diacetate (6)

To a stirred solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-cyanophenyl)acetamido)propane-1,2-diyl diacetate (5) (1.4 g, 1.85 mmol), tert-butyl (22-hydroxy-16-oxo-3,6,9,12-tetraoxa-15-azadocosyl)carbamate (4) (1.48 g, 2.22 mmol), Silver (1+) trifluoro methane sulfonate (AgOTf) (1.15 g, 4.63 mmol) in anhydrous dichloromethane (8 mL) and anhydrous acetonitrile (17 mL) was added activated 4 Å powdered molecular sieves (4 g) and the reaction mixture was stirred at room temperature for 1 h under nitrogen atmosphere. The reaction mixture was cooled to −78° C., iodine monobromide (564 mg, 2.78 mmol) dissolved in dichloromethane (4 mL) was added dropwise and the resulting mixture was stirred at the same temperature for 2 h. After completion, the reaction mixture was quenched with triethylamine (5 mL) and stirred at the room temperature. The reaction mixture was filtered, the filtrate was washed with 10% aqueous sodium thiosulfate solution, dried over sodium sulfate, filtered, concentrated under reduced pressure. The crude residue was purified by C18 reverse phase column chromatography using water and acetonitrile (+0.05% TFA) as eluents to afford the desired product (1R,2R)-1-((2R,3R,4S,6R)-4-acetoxy-3-(2-acetoxyacetamido)-6-((2,2-dimethyl-4,21-dioxo-3,8,11,14,17-pentaoxa-5,20-diazaheptacosan-27-yl)oxy)-6-(methoxycarbonyl)tetrahydro-2H-pyran-2-yl)-3-(2-(4-cyanophenyl)acetamido)propane-1,2-diyl diacetate (6) as a pale yellow solid, Yield: 1.3 g, 64%; LCMS (ESI+) m/z 1096.70 [M+H]⁺.

### Synthesis of (2R,4S,5R,6R)-6-((1R,2R)-3-(2-(4-cyanophenyl) acetamido)-1,2-dihydroxypropyl)-2-((2,2-dimethyl-4,21-dioxo-3,8,11,14,17-pentaoxa-5,20-diazaheptacosan-27-yl)oxy)-4-hydroxy-5-(2-hydroxyacetamido)tetrahydro-2H-pyran-2-carboxylic acid (7)

To a stirred solution (1R,2R)-1-((2R,3R,4S,6R)-4-acetoxy-3-(2-acetoxyacetamido)-6-((2,2-dimethyl-4,21-dioxo-3,8,11,14,17-pentaoxa-5,20-diazaheptacosan-27-yl)oxy)-6-(methoxycarbonyl)tetrahydro-2H-pyran-2-yl)-3-(2-(4-cyanophenyl)acetamido)propane-1,2-diyl diacetate (6) (1.3 g, 1.42 mmol) in methanol (12 mL) and water (3 mL) was added NaOMe (25% in MeOH) (0.42 mL, 7.10 mmol) dropwise at 0° C. The resulting mixture was stirred at room temperature for 16 h. After completion, the reaction mixture was treated with acidic resin (Dowex 50, H⁺) to maintain pH˜6 and the suspension was filtered. The filtrate was concentrated under reduced pressure and the crude residue obtained was purified by C18 reverse phase column chromatography using water and acetonitrile (+0.05% TFA) as eluents to afford the desired product (2R,4S,5R,6R)-6-((1R,2R)-3-(2-(4-cyanophenyl)acetamido)-1,2-dihydroxypropyl)-2-((2,2-dimethyl-4,21-dioxo-3,8,11,14,17-pentaoxa-5,20-diazaheptacosan-27-yl)oxy)-4-hydroxy-5-(2-hydroxyacetamido)tetrahydro-2H-pyran-2-carboxylic acid (7) as an off white solid, Yield: 800 mg, 73%; LCMS (ESI+) m/z 914.60 [M+H]⁺.

### Synthesis of (2R,4S,5R,6R)-6-((1R,2R)-3-(2-(4-(1H-tetrazol-5-yl)phenyl)acetamido)-1,2-dihydroxypropyl)-2-((2,2-dimethyl-4,21-dioxo-3,8,11,14,17-pentaoxa-5,20-diazaheptacosan-27-yl)oxy)-4-hydroxy-5-(2-hydroxyacetamido)tetrahydro-2H-pyran-2-carboxylic acid (8)

To a stirred solution of (2R,4S,5R,6R)-6-((1R,2R)-3-(2-(4-cyanophenyl)acetamido)-1,2-dihydroxypropyl)-2-((2,2-dimethyl-4,21-dioxo-3,8,11,14,17-pentaoxa-5,20-diazaheptacosan-27-yl)oxy)-4-hydroxy-5-(2-hydroxyacetamido)tetrahydro-2H-pyran-2-carboxylic acid (7) (800 mg, 0.88 mmol) in DMF (8 mL) were added ammonium chloride (280 mg, 5.25 mmol) and sodium azide (228 mg, 3.52 mmol) at room temperature and the resulting reaction mixture was stirred at 50° C. for 16 h. After completion, the reaction mixture was concentrated and the crude residue obtained was purified by C18 reverse phase column chromatography using water and acetonitrile (+0.05% TFA) as eluents to afford the desired product (2R,4S,5R,6R)-6-((1R,2R)-3-(2-(4-(1H-tetrazol-5-yl)phenyl)acetamido)-1,2-dihydroxypropyl)-2-((2,2-dimethyl-4,21-dioxo-3,8,11,14,17-pentaoxa-5,20-diazaheptacosan-27-yl)oxy)-4-hydroxy-5-(2-hydroxyacetamido)tetrahydro-2H-pyran-2-carboxylic acid (8) as an yellow solid, Yield: 650 mg, 77%; LCMS (ESI+) m/z 957.8 [M+H]⁺.

### Synthesis of (2R,4S,5R,6R)-6-((1R,2R)-3-(2-(4-(1H-tetrazol-5-yl)phenyl)acetamido)-1,2-dihydroxypropyl)-4-hydroxy-5-(2-hydroxyacetamido)-2-((1,1,1-trifluoro-2,19-dioxo-6,9,12,15-tetraoxa-314,18-diazapentacosan-25-yl)oxy)tetrahydro-2H-pyran-2-carboxylic acid (9)

To a stirred solution of (2R,4S,5R,6R)-6-((1R,2R)-3-(2-(4-(1H-tetrazol-5-yl)phenyl)acetamido)-1,2-dihydroxypropyl)-2-((2,2-dimethyl-4,21-dioxo-3,8,11,14,17-pentaoxa-5,20-diazaheptacosan-27-yl)oxy)-4-hydroxy-5-(2-hydroxyacetamido)tetrahydro-2H-pyran-2-carboxylic acid (8) (650 mg, 0.68 mmol) in anhydrous dichloromethane (6.5 mL) was added trifluoroacetic acid (1 mL) dropwise at 0° C. and the resulting reaction mixture was stirred at room temperature for 3 h. After completion, the reaction mixture was concentrated and the crude residue obtained was purified by prep-HPLC (15-80% acetonitrile in water, +0.05% TFA) to afford the desired product (2R,4S,5R,6R)-6-((1R,2R)-3-(2-(4-(1H-tetrazol-5-yl)phenyl)acetamido)-1,2-dihydroxypropyl)-4-hydroxy-5-(2-hydroxyacetamido)-2-((1,1,1-trifluoro-2,19-dioxo-6,9,12,15-tetraoxa-314,18-diazapentacosan-25-yl)oxy)tetrahydro-2H-pyran-2-carboxylic acid (9) as an yellow solid, Yield: 340 mg, 58%; LCMS (ESI+) m/z 857.50 [M+H]⁺.

### Synthesis of (2R,4S,5R,6R)-6-((1R,2R)-3-(2-(4-(1H-tetrazol-5-yl) phenyl) acetamido)-1,2-dihydroxypropyl)-2-((2,19-dioxo-6,9,12,15-tetraoxa-3,18-diazapentacosan-25-yl) oxy)-4-hydroxy-5-(2-hydroxyacetamido) tetrahydro-2H-pyran-2-carboxylic acid (266)

To a stirred solution of (2R,4S,5R,6R)-6-((1R,2R)-3-(2-(4-(1H-tetrazol-5-yl)phenyl)acetamido)-1,2-dihydroxypropyl)-4-hydroxy-5-(2-hydroxyacetamido)-2-((1,1,1-trifluoro-2,19-dioxo-6,9,12,15-tetraoxa-314,18-diazapentacosan-25-yl)oxy)tetrahydro-2H-pyran-2-carboxylic acid (9) (120 mg, 0.14 mmol) in DMF (1.2 mL) and 2,5-dioxopyrrolidin-1-yl acetate (10) (22 mg, 0.14 mmol) was added triethylamine (0.03 ml, 0.21 mmol) at 0° C. The resulting reaction mixture was stirred at the same temperature for 1 h. After completion, the reaction mixture was purified by prep-HPLC to afford the desired product (2R,4S,5R,6R)-6-((1R,2R)-3-(2-(4-(1H-tetrazol-5-yl)phenyl)acetamido)-1,2-dihydroxypropyl)-2-((2,19-dioxo-6,9,12,15-tetraoxa-3,18-diazapentacosan-25-yl)oxy)-4-hydroxy-5-(2-hydroxyacetamido)tetrahydro-2H-pyran-2-carboxylic acid (266) as a white solid, Yield: 28 mg, 22%; LCMS (ESI+) m/z 899.90 [M+H]⁺. ¹H NMR (400 MHz, MeOD) δ 7.99 (d, J=8 Hz, 2H), 7.55 (d, J=8 Hz, 2H), 4.02 (s, 2H), 3.92-3.62 (m, 20H), 3.56-3.53 (m, 4H), 3.43-3.33 (m, 7H), 2.21-2.17 (m, 2H), 1.96 (s, 3H), 1.76-1.70 (m, 1H), 1.61-1.51 (m, 4H), 1.34-1.31 (m, 5H).

### (2R,4S,5R,6R)-6-((1R,2R)-3-(2-(4-(1H-tetrazol-5-yl)phenyl)acetamido)-1,2-dihydroxypropyl)-2-((1-(2,5-dioxo-2,5-dihydro-1H-pyrrol-1-yl)-3,20-dioxo-7,10,13,16-tetraoxa-4,19-diazahexacosan-26-yl)oxy)-4-hydroxy-5-(2-hydroxyacetamido)tetrahydro-2H-pyran-2-carboxylic acid (Cpd. 266M)

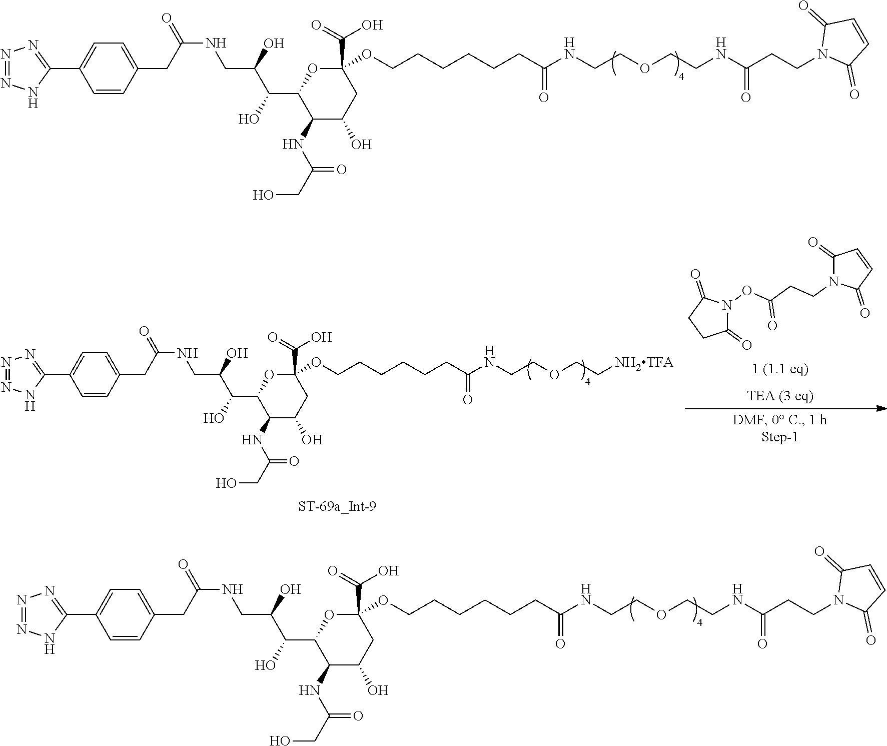

### Synthesis of (2R,4S,5R,6R)-6-((1R,2R)-3-(2-(4-(1H-tetrazol-5-yl)phenyl)acetamido)-1,2-dihydroxypropyl)-2-((1-(2,5-dioxo-2,5-dihydro-1H-pyrrol-1-yl)-3,20-dioxo-7,10,13,16-tetraoxa-4,19-diazahexacosan-26-yl)oxy)-4-hydroxy-5-(2-hydroxyacetamido)tetrahydro-2H-pyran-2-carboxylic acid (266M)

To a stirred solution of (2R,4S,5R,6R)-6-((1R,2R)-3-(2-(4-(1H-tetrazol-5-yl)phenyl)acetamido)-1,2-dihydroxypropyl)-4-hydroxy-5-(2-hydroxyacetamido)-2-((1,1,1-trifluoro-2,19-dioxo-6,9,12,15-tetraoxa-314,18-diazapentacosan-25-yl)oxy)tetrahydro-2H-pyran-2-carboxylic acid (266_Int-9) (120 mg, 0.13 mmol) in DMF (1.2 mL) were added 2,5-dioxopyrrolidin-1-yl 3-(2,5-dioxo-2,5-dihydro-1H-pyrrol-1-yl)propanoate (1) (37 mg, 0.14 mmol) and triethylamine (0.06 mL, 0.39 mmol) at 0° C. The resulting reaction mixture was stirred at the same temperature for 1 h. After completion, the reaction mixture was purified by prep-HPLC to afford the desired product (2R,4S,5R,6R)-6-((1R,2R)-3-(2-(4-(1H-tetrazol-5-yl)phenyl)acetamido)-1,2-dihydroxypropyl)-2-((1-(2,5-dioxo-2,5-dihydro-1H-pyrrol-1-yl)-3,20-dioxo-7,10,13,16-tetraoxa-4,19-diazahexacosan-26-yl)oxy)-4-hydroxy-5-(2-hydroxyacetamido)tetrahydro-2H-pyran-2-carboxylic acid (266M) as a white solid, Yield: 15 mg, 11%; LCMS (ESI+) m/z 1008.70 [M+H]⁺. ¹H NMR (400 MHz, MeOD) δ 7.99 (d, J=8 Hz, 2H), 7.55 (d, J=8.4 Hz, 2H), 6.83 (s, 2H), 4.02 (s, 2H), 3.91-3.62 (m, 21H), 3.56-3.25 (m, 13H), 2.78-2.72 (m, 1H), 2.48 (t, J=6.8 Hz, 2H), 2.21-2.17 (m, 2H), 1.76-1.72 (m, 1H), 1.61-1.52 (m, 4H), 1.32 (d, J=9.6 Hz, 4H), 0.91 (d, J=9.6 Hz, 2H).

### (2R,4S,5R,6R)-6-((1R,2R)-3-(2-(4-(1H-tetrazol-5-yl)phenyl)acetamido)-1,2-dihydroxypropyl)-4-hydroxy-5-(2-hydroxyacetamido)-2-((5,21,38-trioxo-1-((3aR,4R,6aS)-2-oxohexahydro-1H-thieno[3,4-d]imidazol-4-yl)-9,12,15,18,25,28,31,34-octaoxa-6,22,37-triazatetratetracontan-44-yl)oxy)tetrahydro-2H-pyran-2-carboxylic acid (Cpd. 266B)

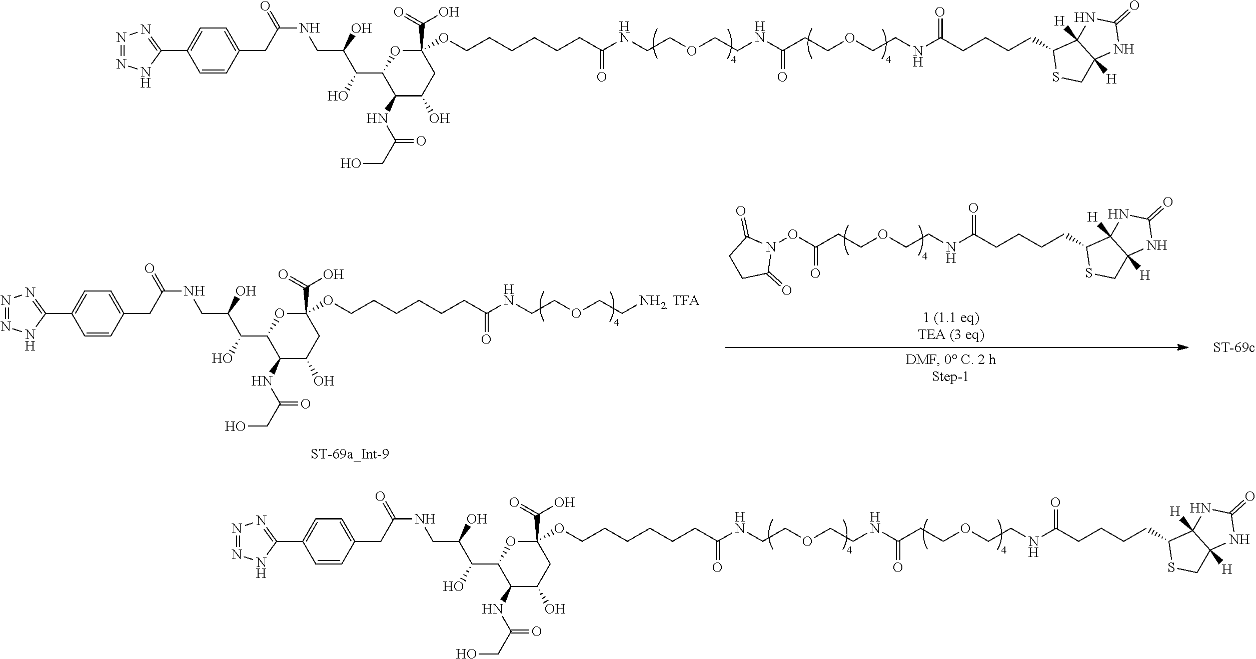

### Synthesis of (2R,4S,5R,6R)-6-((1R,2R)-3-(2-(4-(1H-tetrazol-5-yl)phenyl)acetamido)-1,2-dihydroxypropyl)-4-hydroxy-5-(2-hydroxyacetamido)-2-((5,21,38-trioxo-1-((3aR,4R,6aS)-2-oxohexahydro-1H-thieno[3,4-d]imidazol-4-yl)-9,12,15,18,25,28,31,34-octaoxa-6,22,37-triazatetratetracontan-44-yl)oxy)tetrahydro-2H-pyran-2-carboxylic acid (266B)

To a stirred solution of (2R,4S,5R,6R)-6-((1R,2R)-3-(2-(4-(1H-tetrazol-5-yl)phenyl)acetamido)-1,2-dihydroxypropyl)-4-hydroxy-5-(2-hydroxyacetamido)-2-((1,1,1-trifluoro-2,19-dioxo-6,9,12,15-tetraoxa-314,18-diazapentacosan-25-yl)oxy)tetrahydro-2H-pyran-2-carboxylic acid (266_Int-9) (50 mg, 0.05 mmol) in anhydrous N,N-dimethyl formamide (0.5 mL) were added 2,5-dioxopyrrolidin-1-yl 17-oxo-21-((3aR,4R,6aS)-2-oxohexahydro-1H-thieno[3,4-d]imidazol-4-yl)-4,7,10,13-tetraoxa-16-azahenicosanoate (1) (32 mg, 0.06 mmol) and triethylamine (0.02 ml, 0.15 mmol) at 0° C. The resulting reaction mixture was stirred at the same temperature for 2 h. After completion, the reaction mixture was purified by prep-HPLC using acetonitrile and water (+0.05%, TFA) as eluents to afford the desired product (2R,4S,5R,6R)-6-((1R,2R)-3-(2-(4-(1H-tetrazol-5-yl)phenyl)acetamido)-1,2-dihydroxypropyl)-4-hydroxy-5-(2-hydroxyacetamido)-2-((5,21,38-trioxo-1-((3aR,4R,6aS)-2-oxohexahydro-1H-thieno[3,4-d]imidazol-4-yl)-9,12,15,18,25,28,31,34-octaoxa-6,22,37-triazatetratetracontan-44-yl)oxy)tetrahydro-2H-pyran-2-carboxylic acid (266B) as white solid, Yield: 15 mg, 23%; LCMS (ESI−) m/z 1329.8 [M−H]−. ¹H NMR (400 MHz, MeOD) δ 7.98 (d, J=8.4 Hz, 2H), 7.55 (d, J=8 Hz, 2H), 4.50-4.31 (m, 2H), 4.03 (s, 2H), 3.90-3.71 (m, 6H), 3.65-3.62 (m, 28H), 3.54-3.21 (m, 16H), 2.98-2.92 (m, 1H), 2.73-2.70 (m, 2H), 2.46 (t, J=6 Hz, 2H), 2.24-2.17 (m, 4H), 1.73-1.33 (m, 16H).

### (2R,4S,5R,6R)-6-((1R,2R)-3-(2-(4-(1H-tetrazol-5-yl)phenyl)acetamido)-1,2-dihydroxypropyl)-2-((6-((3,4-dioxo-2-((2-oxo-6,9,12,15-tetraoxa-3-azaheptadecan-17-yl)amino)cyclobut-1-en-1-yl)amino)hexyl)oxy)-4-hydroxy-5-(2-hydroxyacetamido)tetrahydro-2H-pyran-2-carboxylic acid (Cpd. 265)

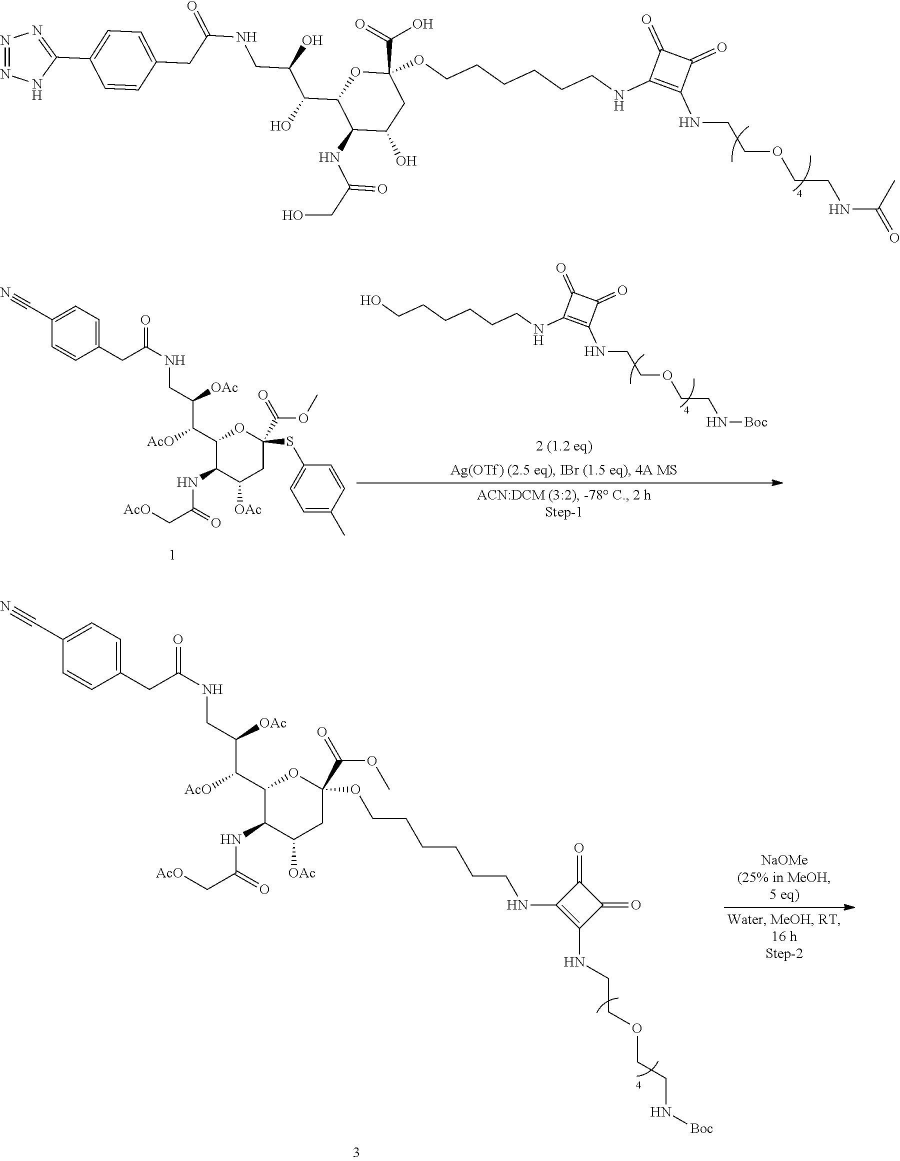

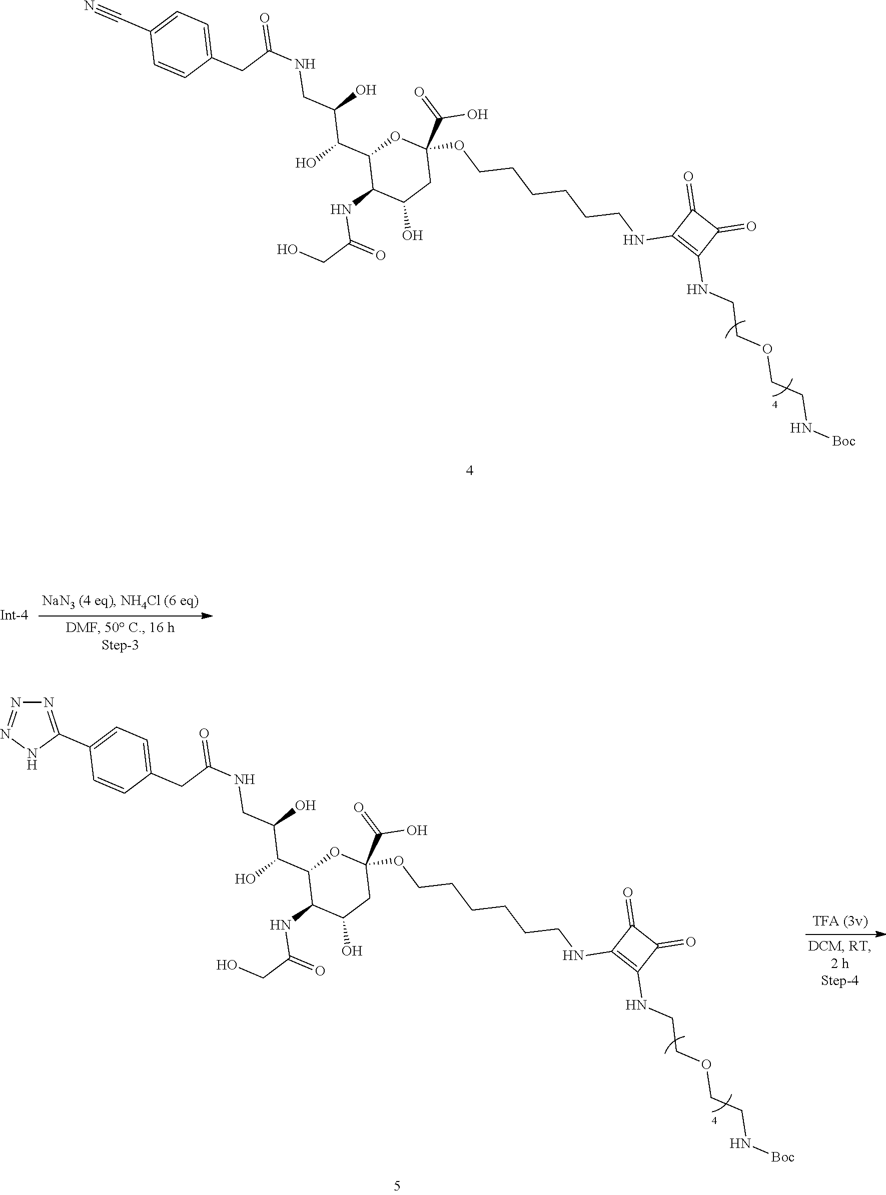

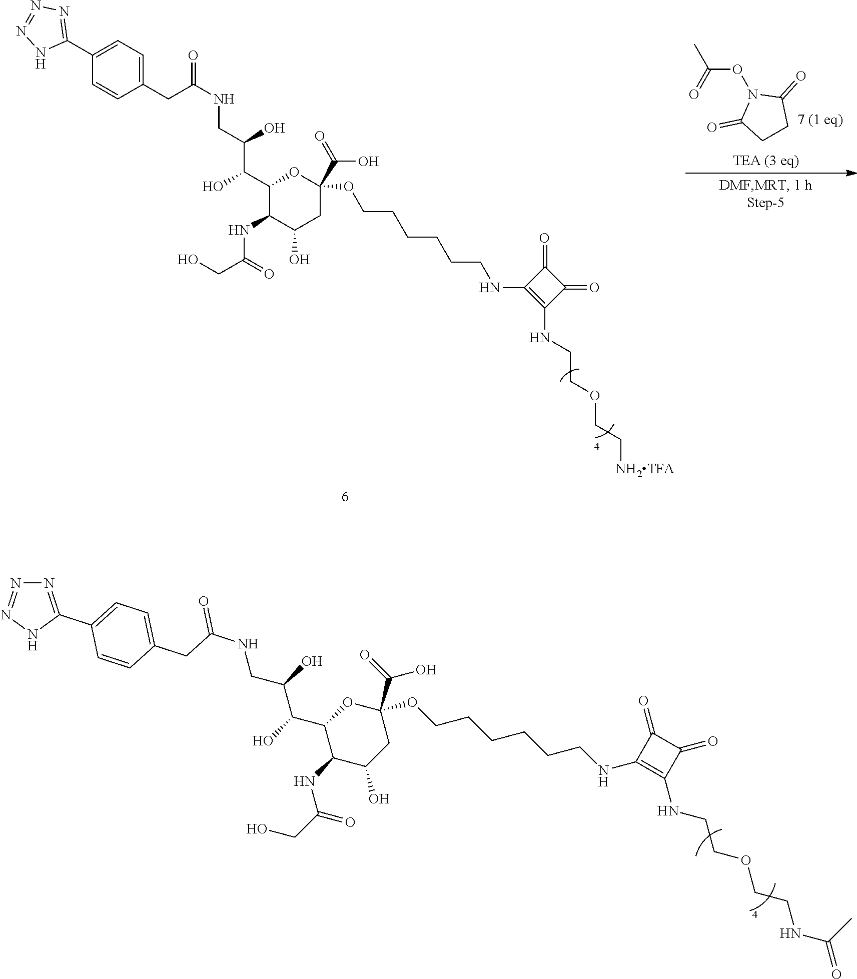

### Synthesis of (1R,2R)-1-((2R,3R,4S,6R)-4-acetoxy-3-(2-acetoxyacetamido)-6-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-6-(methoxycarbonyl)tetrahydro-2H-pyran-2-yl)-3-(2-(4-cyanophenyl)acetamido)propane-1,2-diyl diacetate (3)

To a stirred solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-cyanophenyl)acetamido)propane-1,2-diyl diacetate (1) (2 g, 2.64 mmol), tert-butyl (14-((2-((6-hydroxyhexyl)amino)-3,4-dioxocyclobut-1-en-1-yl)amino)-3,6,9,12-tetraoxatetradecyl)carbamate (2) (1.69 g, 3.18 mmol), Silver (1+) trifluoro methane sulfonate (AgOTf) (1.69 g, 6.60 mmol) in anhydrous dichloromethane (12 mL) and anhydrous acetonitrile (24 mL) was added activated 4 Å powdered molecular sieves (5 g) and the reaction mixture was stirred at room temperature for 1 h under nitrogen atmosphere. The solution was cooled to −78° C., iodine monobromide (819 mg, 3.96 mmol) dissolved in dichloromethane (4 mL) was added dropwise and the resulting mixture was stirred at the same temperature for 2 h. After completion, the reaction mixture was quenched with triethylamine (5 mL) and stirred at room temperature. The reaction mixture was filtered, the filtrate was washed with 10% aqueous sodium thiosulfate solution, dried over sodium sulfate, filtered, concentrated under reduced pressure. The crude residue was purified by C18 reverse phase column chromatography using water and acetonitrile (+0.05% TFA) as eluents to afford the desired product (1R,2R)-1-((2R,3R,4S,6R)-4-acetoxy-3-(2-acetoxyacetamido)-6-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-6-(methoxycarbonyl)tetrahydro-2H-pyran-2-yl)-3-(2-(4-cyanophenyl)acetamido)propane-1,2-diyl diacetate (3) as a pale yellow solid, Yield: 1.3 g, 42%; LCMS (ESI+) m/z 1164.0 [M+H]⁺.

### Synthesis of (2R,4S,5R,6R)-6-((1R,2R)-3-(2-(4-cyanophenyl)acetamido)-1,2-dihydroxypropyl)-2-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-4-hydroxy-5-(2-hydroxyacetamido)tetrahydro-2H-pyran-2-carboxylic acid (4)

To a stirred solution (1R,2R)-1-((2R,3R,4S,6R)-4-acetoxy-3-(2-acetoxyacetamido)-6-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-6-(methoxycarbonyl)tetrahydro-2H-pyran-2-yl)-3-(2-(4-cyanophenyl)acetamido)propane-1,2-diyl diacetate (3) (1.3 g, 1.32 mmol) in methanol (12 mL) and water (3 mL) was added NaOMe (25% in MeOH) (0.42 mL, 6.62 mmol) dropwise at 0° C. The resulting reaction mixture was stirred at room temperature for 16 h. After completion, the reaction mixture was treated with acidic resin (Dowex 50, H⁺) to maintain pH˜6 and the suspension was filtered. The filtrate was concentrated under reduced pressure and the crude residue obtained was purified by C18 reverse phase column chromatography using water and acetonitrile (+0.05% TFA) as eluents to afford the desired product (2R,4S,5R,6R)-6-((1R,2R)-3-(2-(4-cyanophenyl)acetamido)-1,2-dihydroxypropyl)-2-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-4-hydroxy-5-(2-hydroxyacetamido)tetrahydro-2H-pyran-2-carboxylic acid (4) as an off white solid, Yield: 700 mg, 63%; LCMS (ESI−) m/z 979.70 [M−H]−.

### Synthesis of (2R,4S,5R,6R)-6-((1R,2R)-3-(2-(4-(1H-tetrazol-5-yl)phenyl)acetamido)-1,2-dihydroxypropyl)-2-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-4-hydroxy-5-(2-hydroxyacetamido)tetrahydro-2H-pyran-2-carboxylic acid (5)

To a stirred solution of (2R,4S,5R,6R)-6-((1R,2R)-3-(2-(4-cyanophenyl)acetamido)-1,2-dihydroxypropyl)-2-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-4-hydroxy-5-(2-hydroxyacetamido)tetrahydro-2H-pyran-2-carboxylic acid (4) (700 mg, 0.71 mmol) in DMF (7 mL) were added ammonium chloride (228 mg, 4.28 mmol), sodium azide (185 mg, 2.84 mmol) and the resulting reaction mixture was stirred at 50° C. for 16 h. After completion, the reaction mixture was concentrated and the crude residue obtained was purified by C18 reverse phase column chromatography using water and acetonitrile (+0.05% TFA) as eluents to afford the desired product (2R,4S,5R,6R)-6-((1R,2R)-3-(2-(4-(1H-tetrazol-5-yl)phenyl)acetamido)-1,2-dihydroxypropyl)-2-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-4-hydroxy-5-(2-hydroxyacetamido)tetrahydro-2H-pyran-2-carboxylic acid (5) as an yellow solid, Yield: 500 mg, 68%; LCMS (ESI+) m/z 1024.7 [M+H]⁺.

### Synthesis of (2R,4S,5R,6R)-6-((1R,2R)-3-(2-(4-(1H-tetrazol-5-yl)phenyl)acetamido)-1,2-dihydroxypropyl)-2-((6-((3,4-dioxo-2-((1,1,1-trifluoro-2-oxo-6,9,12,15-tetraoxa-314-azaheptadecan-17-yl)amino)cyclobut-1-en-1-yl)amino)hexyl)oxy)-4-hydroxy-5-(2-hydroxyacetamido)tetrahydro-2H-pyran-2-carboxylic acid (6)

To a stirred solution of (2R,4S,5R,6R)-6-((1R,2R)-3-(2-(4-(1H-tetrazol-5-yl)phenyl)acetamido)-1,2-dihydroxypropyl)-2-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-4-hydroxy-5-(2-hydroxyacetamido)tetrahydro-2H-pyran-2-carboxylic acid (5) (500 mg, 0.49 mmol) in anhydrous dichloromethane (5 mL) was added trifluoroacetic acid (1.5 mL) dropwise at 0° C. and the resulting reaction mixture was stirred at room temperature for 3 h. After completion, the reaction mixture was concentrated and the crude residue obtained was purified by C18 reverse phase column chromatography using water and acetonitrile (+0.05% TFA) to afford the desired product (2R,4S,5R,6R)-6-((1R,2R)-3-(2-(4-(1H-tetrazol-5-yl)phenyl)acetamido)-1,2-dihydroxypropyl)-2-((6-((3,4-dioxo-2-((1,1,1-trifluoro-2-oxo-6,9,12,15-tetraoxa-314-azaheptadecan-17-yl)amino)cyclobut-1-en-1-yl)amino)hexyl)oxy)-4-hydroxy-5-(2-hydroxyacetamido)tetrahydro-2H-pyran-2-carboxylic acid (6) as an yellow solid, Yield: 380 mg, 76%; LCMS (ESI+) m/z 924.90 [M+H]⁺.

### Synthesis of (2R,4S,5R,6R)-6-((1R,2R)-3-(2-(4-(1H-tetrazol-5-yl)phenyl)acetamido)-1,2-dihydroxypropyl)-2-((6-((3,4-dioxo-2-((2-oxo-6,9,12,15-tetraoxa-3-azaheptadecan-17-yl)amino)cyclobut-1-en-1-yl)amino)hexyl)oxy)-4-hydroxy-5-(2-hydroxyacetamido)tetrahydro-2H-pyran-2-carboxylic acid (265)

To a stirred solution of (2R,4S,5R,6R)-6-((1R,2R)-3-(2-(4-(1H-tetrazol-5-yl)phenyl)acetamido)-1,2-dihydroxypropyl)-2-((6-((3,4-dioxo-2-((1,1,1-trifluoro-2-oxo-6,9,12,15-tetraoxa-314-azaheptadecan-17-yl)amino)cyclobut-1-en-1-yl)amino)hexyl)oxy)-4-hydroxy-5-(2-hydroxyacetamido)tetrahydro-2H-pyran-2-carboxylic acid (6) (120 mg, 0.12 mmol) in DMF (1.2 mL) and 2,5-dioxopyrrolidin-1-yl acetate (7) (19 mg, 0.12 mmol) was added triethylamine (0.05 ml, 0.36 mmol) at 0° C. The resulting reaction mixture was stirred at the same temperature for 1 h. After completion, the reaction mixture was purified by prep-HPLC to afford the desired product (2R,4S,5R,6R)-6-((1R,2R)-3-(2-(4-(1H-tetrazol-5-yl)phenyl)acetamido)-1,2-dihydroxypropyl)-2-((6-((3,4-dioxo-2-((2-oxo-6,9,12,15-tetraoxa-3-azaheptadecan-17-yl)amino)cyclobut-1-en-1-yl)amino)hexyl)oxy)-4-hydroxy-5-(2-hydroxyacetamido)tetrahydro-2H-pyran-2-carboxylic acid (265) as a white solid, Yield: 38 mg, 33%; LCMS (ESI+) m/z 966.50 [M+H]⁺. ¹H NMR (400 MHz, MeOD) δ 7.99 (d, J=8.4 Hz, 2H), 7.55 (d, J=8 Hz, 2H), 4.02 (s, 2H), 3.91-3.61 (m, 23H), 3.56-3.26 (m, 9H), 2.76-2.71 (m, 1H), 1.96 (s, 3H), 1.73-1.31 (m, 11H).

### (2R,4S,5R,6R)-6-((1R,2R)-3-(2-(4-(1H-tetrazol-5-yl)phenyl)acetamido)-1,2-dihydroxypropyl)-2-((6-((2-((18-(2,5-dioxo-2,5-dihydro-1H-pyrrol-1-yl)-16-oxo-3,6,9,12-tetraoxa-15-azaoctadecyl)amino)-3,4-dioxocyclobut-1-en-1-yl)amino)hexyl)oxy)-4-hydroxy-5-(2-hydroxyacetamido)tetrahydro-2H-pyran-2-carboxylic acid (Cpd. 265M)

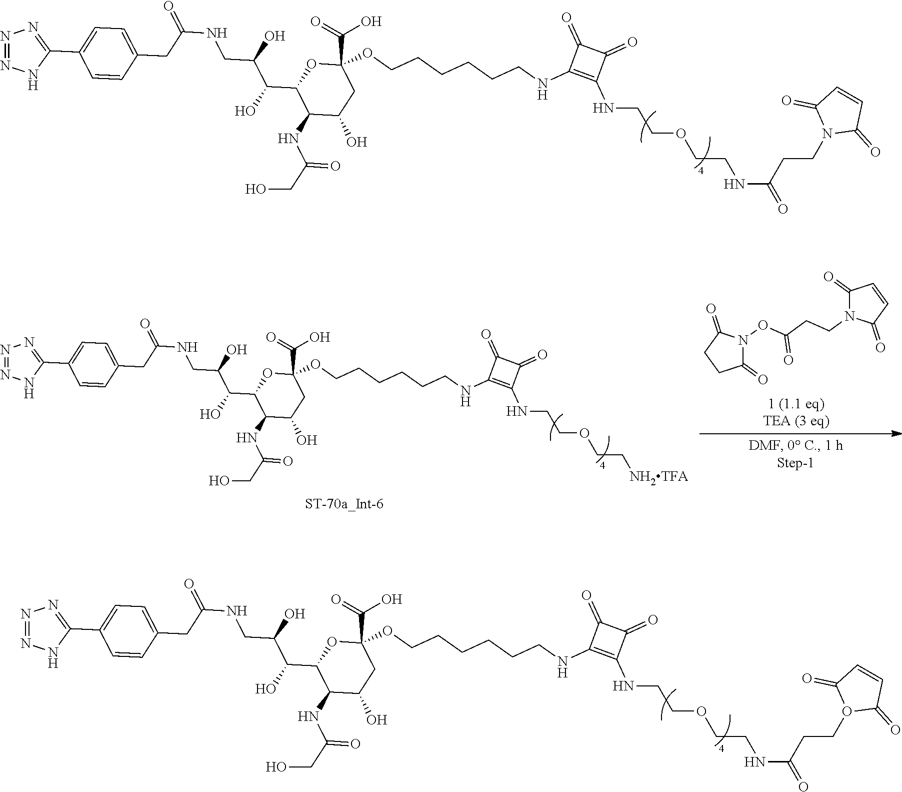

### Synthesis of (2R,4S,5R,6R)-6-((1R,2R)-3-(2-(4-(1H-tetrazol-5-yl)phenyl)acetamido)-1,2-dihydroxypropyl)-2-((6-((2-((18-(2,5-dioxo-2,5-dihydro-1H-pyrrol-1-yl)-16-oxo-3,6,9,12-tetraoxa-15-azaoctadecyl)amino)-3,4-dioxocyclobut-1-en-1-yl)amino)hexyl)oxy)-4-hydroxy-5-(2-hydroxyacetamido)tetrahydro-2H-pyran-2-carboxylic acid (265M)

To a stirred solution of (2R,4S,5R,6R)-6-((1R,2R)-3-(2-(4-(1H-tetrazol-5-yl)phenyl)acetamido)-1,2-dihydroxypropyl)-2-((6-((3,4-dioxo-2-((1,1,1-trifluoro-2-oxo-6,9,12,15-tetraoxa-314-azaheptadecan-17-yl)amino)cyclobut-1-en-1-yl)amino)hexyl)oxy)-4-hydroxy-5-(2-hydroxyacetamido)tetrahydro-2H-pyran-2-carboxylic acid (265_Int-6) (120 mg, 0.12 mmol) in DMF (1.2 mL) were added 2,5-dioxopyrrolidin-1-yl 3-(2,5-dioxo-2,5-dihydro-1H-pyrrol-1-yl)propanoate (1) (35 mg, 0.13 mmol) and triethylamine (0.05 mL, 0.36 mmol) at 0° C. The resulting reaction mixture was stirred at the same temperature for 1 h. After completion, the reaction mixture was purified by prep-HPLC to afford the desired product (2R,4S,5R,6R)-6-((1R,2R)-3-(2-(4-(1H-tetrazol-5-yl)phenyl)acetamido)-1,2-dihydroxypropyl)-2-((6-((2-((18-(2,5-dioxo-2,5-dihydro-1H-pyrrol-1-yl)-16-oxo-3,6,9,12-tetraoxa-15-azaoctadecyl)amino)-3,4-dioxocyclobut-1-en-1-yl)amino)hexyl)oxy)-4-hydroxy-5-(2-hydroxyacetamido)tetrahydro-2H-pyran-2-carboxylic acid (265M) as a white solid, Yield: 19 mg, 15%; LCMS (ESI+) m/z 1075.70 [M+H]⁺. ¹H NMR (400 MHz, MeOD) δ 7.99 (d, J=7.6 Hz, 2H), 7.56 (d, J=8 Hz, 2H), 6.83 (s, 2H), 4.05 (s, 2H), 4.04-3.64 (m, 21H), 3.63-3.15 (m, 14H), 2.74-2.70 (m, 1H), 2.48 (t, J=6.8 Hz, 2H), 1.73 (t, J=12 Hz, 1H), 1.59-0.89 (m, 10H).

### (2R,4S,5R,6R)-6-((1R,2R)-3-(2-(4-(1H-tetrazol-5-yl)phenyl)acetamido)-1,2-dihydroxypropyl)-2-((6-((2-((16,32-dioxo-36-((3aR,4R,6aS)-2-oxohexahydro-1H-thieno[3,4-d]imidazol-4-yl)-3,6,9,12,19,22,25,28-octaoxa-15,31-diazahexatriacontyl)amino)-3,4-dioxocyclobut-1-en-1-yl)amino)hexyl)oxy)-4-hydroxy-5-(2-hydroxyacetamido)tetrahydro-2H-pyran-2-carboxylic acid (Cpd. 265B)

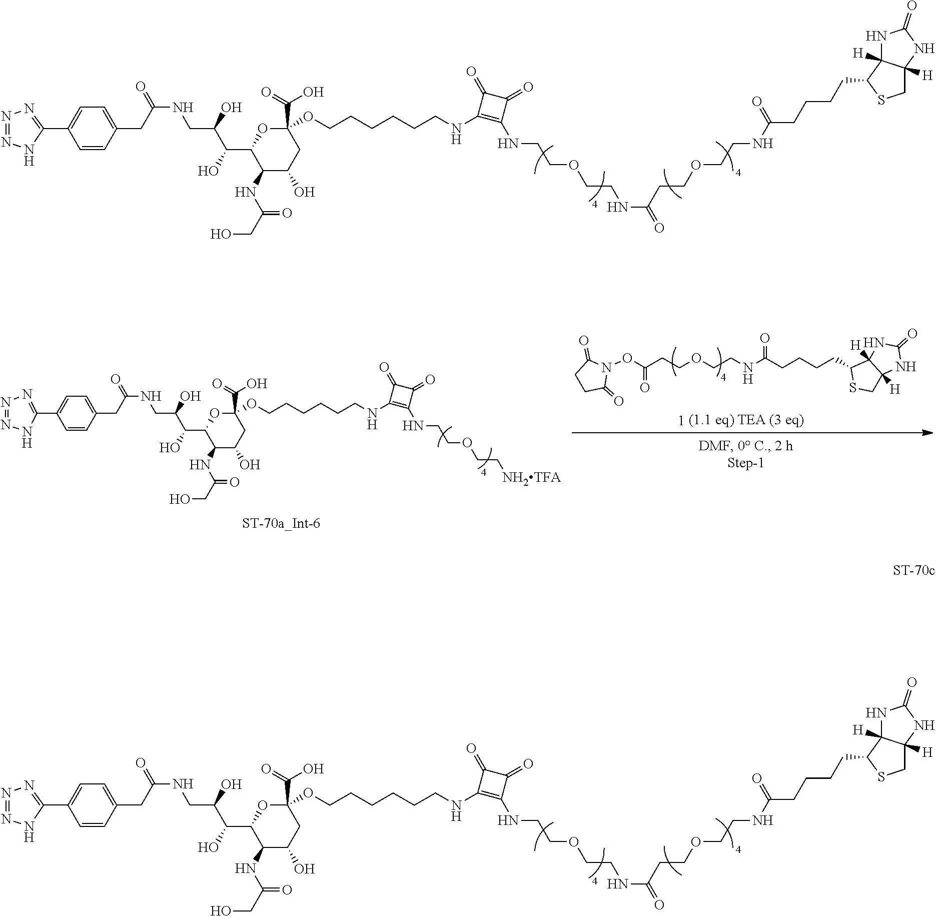

### Synthesis of (2R,4S,5R,6R)-6-((1R,2R)-3-(2-(4-(1H-tetrazol-5-yl)phenyl)acetamido)-1,2-dihydroxypropyl)-2-((6-((2-((16,32-dioxo-36-((3aR,4R,6aS)-2-oxohexahydro-1H-thieno[3,4-d]imidazol-4-yl)-3,6,9,12,19,22,25,28-octaoxa-15,31-diazahexatriacontyl)amino)-3,4-dioxocyclobut-1-en-1-yl)amino)hexyl)oxy)-4-hydroxy-5-(2-hydroxyacetamido)tetrahydro-2H-pyran-2-carboxylic acid (265B)

To a stirred solution of (2R,4S,5R,6R)-6-((1R,2R)-3-(2-(4-(1H-tetrazol-5-yl)phenyl)acetamido)-1,2-dihydroxypropyl)-2-((6-((3,4-dioxo-2-((1,1,1-trifluoro-2-oxo-6,9,12,15-tetraoxa-314-azaheptadecan-17-yl)amino)cyclobut-1-en-1-yl)amino)hexyl)oxy)-4-hydroxy-5-(2-hydroxyacetamido)tetrahydro-2H-pyran-2-carboxylic acid (265_Int-6) (50 mg, 0.04 mmol) in anhydrous N,N-dimethyl formamide (0.5 mL) were added 2,5-dioxopyrrolidin-1-yl 17-oxo-21-((3aR,4R,6aS)-2-oxohexahydro-1H-thieno[3,4-d]imidazol-4-yl)-4,7,10,13-tetraoxa-16-azahenicosanoate (1) (26 mg, 0.04 mmol) and triethylamine (0.02 ml, 0.12 mmol) at 0° C. The resulting reaction mixture was stirred at the same temperature for 2 h. After completion, the reaction mixture was purified by prep-HPLC using acetonitrile and water (+0.05%, TFA) as eluents to afford the desired product (2R,4S,5R,6R)-6-((1R,2R)-3-(2-(4-(1H-tetrazol-5-yl)phenyl)acetamido)-1,2-dihydroxypropyl)-2-((6-((2-((16,32-dioxo-36-((3aR,4R,6aS)-2-oxohexahydro-1H-thieno[3,4-d]imidazol-4-yl)-3,6,9,12,19,22,25,28-octaoxa-15,31-diazahexatriacontyl)amino)-3,4-dioxocyclobut-1-en-1-yl)amino)hexyl)oxy)-4-hydroxy-5-(2-hydroxyacetamido)tetrahydro-2H-pyran-2-carboxylic acid (265B) as white solid, Yield: 20 mg, 36%; LCMS (ESI−) m/z 1397.9 [M−H]⁻. ¹H NMR (400 MHz, MeOD) δ 7.99 (d, J=8.4 Hz, 2H), 7.56 (d, J=8.4 Hz, 2H), 4.53-4.31 (m, 2H), 4.03 (s, 2H), 3.83-3.70 (m, 11H), 3.68-3.63 (m, 30H), 3.57-3.23 (m, 11H), 2.98-2.92 (m, 1H), 2.74-2.70 (m, 2H), 2.48 (t, J=6 Hz, 2H), 2.25-2.22 (m, 2H), 1.78-1.26 (m, 16H).

### (2R,4S,5R,6R)-6-((1R,2R)-3-(2-(4-chlorophenyl) acetamido)-1,2-dihydroxypropyl)-2-((2,19-dioxo-6,9,12,15-tetraoxa-3,18-diazapentacosan-25-yl) oxy)-4-hydroxy-5-(2-hydroxyacetamido) tetrahydro-2H-pyran-2-carboxylic acid (Cpd. 264)

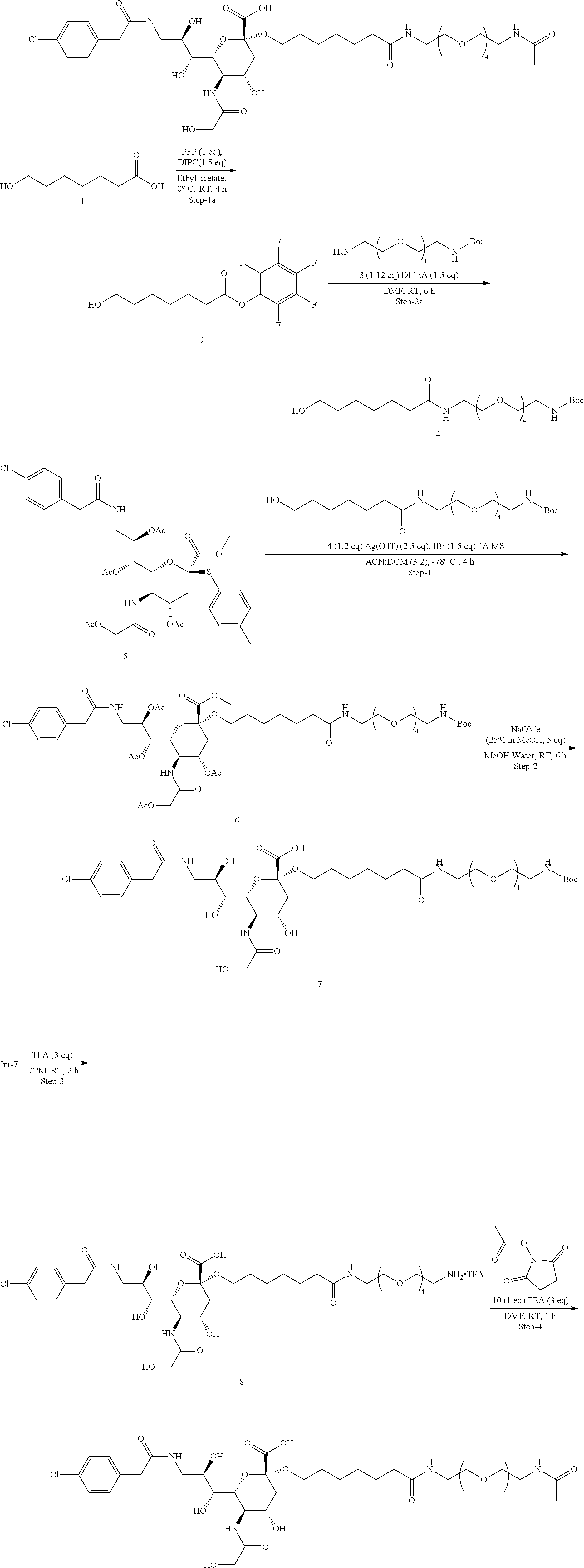

### Synthesis of perfluorophenyl 7-hydroxyheptanoate (2)

To a stirred solution of 7-hydroxyheptanoic acid (1) (5 g, 34 mmol) in EtOAc (50 mL) was added 2,3,4,5,6-pentafluorophenol (6.25 g, 34 mmol), followed by dropwise addition of N, N′-Di-isopropyl carbodiimide (6.43 g, 51 mmol) at 0° C. The resulting reaction mixture was stirred for 4 h at the same temperature. After completion, the reaction mixture was filtered, the residue was washed with ethyl acetate, and the filtrate was concentrated under reduced pressure to afford the desired product perfluorophenyl 7-hydroxyheptanoate (2), which was used as such for the next step, Yield: 7 g, 65%.

### Synthesis of tert-butyl (22-hydroxy-16-oxo-3,6,9,12-tetraoxa-15-azadocosyl) carbamate (4)

To a stirred solution of perfluorophenyl 7-hydroxyheptanoate (2) (7 g, 22 mmol) in DMF (70 mL) were added tert-butyl (14-amino-3,6,9,12-tetraoxatetradecyl)carbamate (4) (7.5 g, 22 mmol), DIPEA (6 mL, 33 mmol) at 0° C. and the reaction mixture was stirred at the same temperature for 2 h. After completion, the reaction mixture was purified by C18 reverse phase column chromatography using water and acetonitrile (+0.05% TFA) as eluents to afford the desired product tert-butyl (22-hydroxy-16-oxo-3,6,9,12-tetraoxa-15-azadocosyl) carbamate (4) as off-white solid. Yield: 7 g, 67%; LCMS (ESI+) m/z 465.5 [M+H]⁺.

### Synthesis of (1R,2R)-1-((2R,3R,4S,6R)-4-acetoxy-3-(2-acetoxyacetamido)-6-((2,2-dimethyl-4,21-dioxo-3,8,11,14,17-pentaoxa-5,20-diazaheptacosan-27-yl)oxy)-6-(methoxycarbonyl)tetrahydro-2H-pyran-2-yl)-3-(2-(4-chlorophenyl)acetamido)propane-1,2-diyl diacetate (6)

To a stirred solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (5) (2 g, 2.61 mmol), tert-butyl (22-hydroxy-16-oxo-3,6,9,12-tetraoxa-15-azadocosyl)carbamate (4) (1.45 g, 3.13 mmol), Silver (1+) trifluoro methane sulfonate (AgOTf) (1.67 g, 6.52 mmol) in anhydrous dichloromethane (24 mL) and anhydrous acetonitrile (36 mL) was added activated 4 Å powdered molecular sieves (4 g) and the reaction mixture was stirred at room temperature for 1 h under nitrogen atmosphere. The reaction mixture was cooled to −78° C., iodine monobromide (807 mg, 3.92 mmol) dissolved in dichloromethane (4 mL) was added dropwise and the resulting mixture was stirred at the same temperature for 2 h. After completion, the reaction mixture was quenched with triethylamine (5 mL) and stirred at the room temperature. The reaction mixture was filtered, the filtrate was washed with 10% aqueous sodium thiosulfate solution, dried over sodium sulfate, filtered, concentrated under reduced pressure. The crude residue was purified by C18 reverse phase column chromatography using water and acetonitrile (+0.05% TFA) as eluents to afford the desired product (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (6) as off-white solid, Yield-1.3 g, 44.99%; LCMS (ESI+) m/z 1105.4 [M+H]⁺.

### Synthesis of (2R,4S,5R,6R)-6-((1R,2R)-3-(2-(4-chlorophenyl) acetamido)-1,2-dihydroxypropyl)-2-((2,2-dimethyl-4,21-dioxo-3,8,11,14,17-pentaoxa-5,20-diazaheptacosan-27-yl) oxy)-4-hydroxy-5-(2-hydroxyacetamido) tetrahydro-2H-pyran-2-carboxylic acid (7)

To a stirred solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (6) (1.3 g, 1.17 mmol) in methanol (7 mL) and water (3 mL) was added 25% sodium methoxide in methanol (0.4 mL, 5.88 mmol) at 0° C. and the reaction mixture was stirred at room temperature for 16 h. After completion the reaction mixture was treated with acidic resin (Dowex 50, H⁺) to make the pH˜6 and the suspension was filtered, filtrate was concentrated to afford the desired product (2R,4S,5R,6R)-6-((1R,2R)-3-(2-(4-chlorophenyl)acetamido)-1,2-dihydroxypropyl)-2-((2,2-dimethyl-4,21-dioxo-3,8,11,14,17-pentaoxa-5,20-diazaheptacosan-27-yl)oxy)-4-hydroxy-5-(2-hydroxyacetamido)tetrahydro-2H-pyran-2-carboxylic acid (7) as off-white solid, Yield: 700 mg, 64.46%; LCMS (ESI+) m/z 921.4 [M−H]⁻.

### Synthesis of (2R,4S,5R,6R)-6-((1R,2R)-3-(2-(4-chlorophenyl) acetamido)-1,2-dihydroxypropyl)-4-hydroxy-5-(2-hydroxyacetamido)-2-((1,1,1-trifluoro-2,19-dioxo-6,9,12,15-tetraoxa-314,18-diazapentacosan-25-yl) oxy) tetrahydro-2H-pyran-2-carboxylic acid (8)

To a stirred solution of (2R,4S,5R,6R)-6-((1R,2R)-3-(2-(4-chlorophenyl)acetamido)-1,2-dihydroxypropyl)-2-((2,2-dimethyl-4,21-dioxo-3,8,11,14,17-pentaoxa-5,20-diazaheptacosan-27-yl)oxy)-4-hydroxy-5-(2-hydroxyacetamido)tetrahydro-2H-pyran-2-carboxylic acid (7) (0.7 g, 0.76 mmol) in anhydrous dichloromethane (4.5 mL) was added trifluoro acetic acid (1 mL) at 0° C. The reaction mixture was stirred at room temperature for 2 h. After completion, the reaction mixture was concentrated to get the crude which was dissolved in methanol and neutralized with Tetra alkyl ammonium carbonate, the polymer-bound resin. The reaction mixture was filtered and the filtrate was concentrated to afford the desired product (2R,4S,5R,6R)-6-((1R,2R)-3-(2-(4-chlorophenyl)acetamido)-1,2-dihydroxypropyl)-4-hydroxy-5-(2-hydroxyacetamido)-2-((1,1,1-trifluoro-2,19-dioxo-6,9,12,15-tetraoxa-314,18-diazapentacosan-25-yl)oxy)tetrahydro-2H-pyran-2-carboxylic acid (8) as off-white solid, Yield-400 mg, 64.09%; LCMS (ESI+) m/z 823.4 [M+H]⁺.

### Synthesis of (2R,4S,5R,6R)-6-((1R,2R)-3-(2-(4-chlorophenyl) acetamido)-1,2-dihydroxypropyl)-2-((2,19-dioxo-6,9,12,15-tetraoxa-3,18-diazapentacosan-25-yl) oxy)-4-hydroxy-5-(2-hydroxyacetamido) tetrahydro-2H-pyran-2-carboxylic acid (264)

To a stirred solution of (2R,4S,5R,6R)-6-((1R,2R)-3-(2-(4-chlorophenyl)acetamido)-1,2-dihydroxypropyl)-4-hydroxy-5-(2-hydroxyacetamido)-2-((1,1,1-trifluoro-2,19-dioxo-6,9,12,15-tetraoxa-314,18-diazapentacosan-25-yl)oxy)tetrahydro-2H-pyran-2-carboxylic acid (8) (100 mg, 0.12 mmol) in N,N-dimethyl formamide (1 mL) was added 2,5-dioxopyrrolidin-1-yl acetate (10) (19.1 mg, 0.12 mmol) at 0° C. The resulting reaction mixture was stirred at room temperature for 1 h. After completion, the reaction mixture was concentrated and purified by prep-HPLC using acetonitrile and water (+0.05%, TFA) to afford the desired product (2R,4S,5R,6R)-6-((1R,2R)-3-(2-(4-chlorophenyl)acetamido)-1,2-dihydroxypropyl)-2-((2,19-dioxo-6,9,12,15-tetraoxa-3,18-diazapentacosan-25-yl)oxy)-4-hydroxy-5-(2-hydroxyacetamido)tetrahydro-2H-pyran-2-carboxylic acid (264) as white solid, Yield: 70 mg, 66.60%; LCMS (ESI+) m/z 865.8 [M+H]⁺. ¹H NMR (400 MHz, MeOD): δ 7.31 (s, 4H), 4.03 (s, 2H), 3.88-3.63 (m, 18H), 3.55-3.53 (m, 8H), 3.37-3.21 (m, 5H), 2.71 (dd, J=16.4, 8.4 Hz, 1H), 2.21-2.17 (m, 2H), 1.96 (s, 3H), 1.76-1.70 (m, 1H), 1.61-1.56 (m, 4H), 1.34-1.31 (m, 4H).

### (2R,4S,5R,6R)-6-((1R,2R)-3-(2-(4-chlorophenyl)acetamido)-1,2-dihydroxypropyl)-2-((1-(2,5-dioxo-2,5-dihydro-1H-pyrrol-1-yl)-3,20-dioxo-7,10,13,16-tetraoxa-4,19-diazahexacosan-26-yl)oxy)-4-hydroxy-5-(2-hydroxyacetamido)tetrahydro-2H-pyran-2-carboxylic acid (Cpd. 264M)

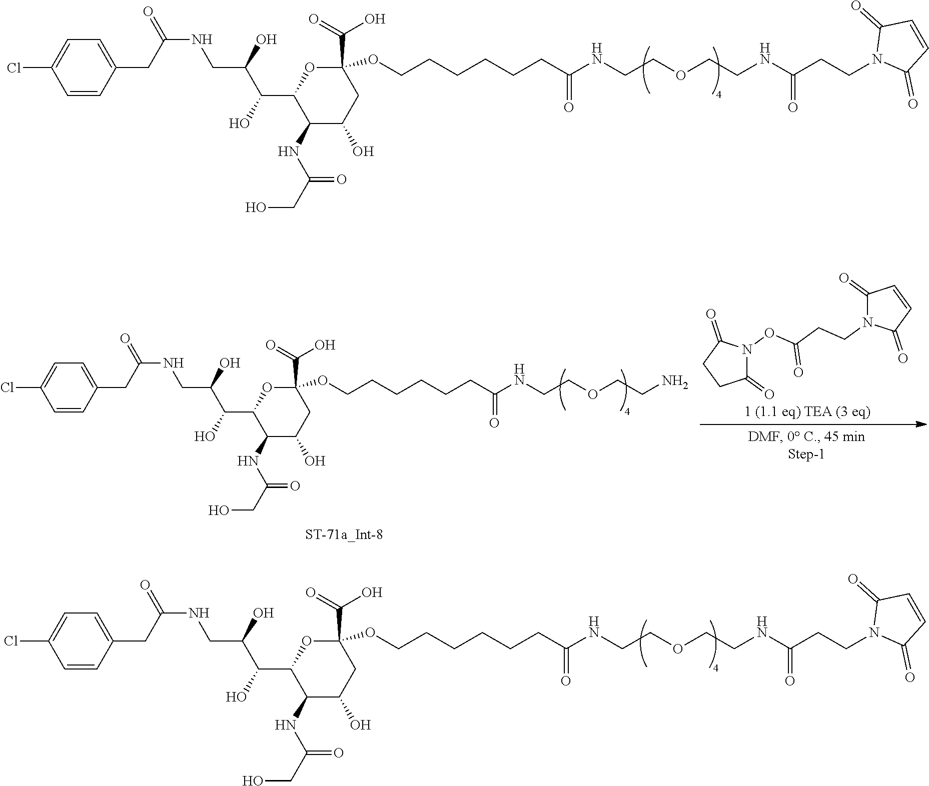

### Synthesis of (2R,4S,5R,6R)-6-((1R,2R)-3-(2-(4-chlorophenyl)acetamido)-1,2-dihydroxypropyl)-2-((1-(2,5-dioxo-2,5-dihydro-1H-pyrrol-1-yl)-3,20-dioxo-7,10,13,16-tetraoxa-4,19-diazahexacosan-26-yl)oxy)-4-hydroxy-5-(2-hydroxyacetamido)tetrahydro-2H-pyran-2-carboxylic acid (264M)

To a stirred solution of (2R,4S,5R,6R)-2-((1-amino-16-oxo-3,6,9,12-tetraoxa-15-azadocosan-22-yl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (264_Int-8) (200 mg, 0.24 mmol) in N,N-dimethyl formamide (2 mL) were added 2,5-dioxopyrrolidin-1-yl 3-(2,5-dioxo-2,5-dihydro-1H-pyrrol-1-yl)propanoate (1) (71.1 mg, 0.27 mmol) and triethylamine (0.1 mL, 0.73 mmol) at 0° C. The resulting reaction mixture was stirred at the same temperature for 45 min. After completion, the reaction mixture was concentrated and purified by prep-HPLC using acetonitrile and water (+0.05%, TFA) as eluents to (2R,4S,5R,6R)-6-((1R,2R)-3-(2-(4-chlorophenyl)acetamido)-1,2-dihydroxypropyl)-2-((1-(2,5-dioxo-2,5-dihydro-1H-pyrrol-1-yl)-3,20-dioxo-7,10,13,16-tetraoxa-4,19-diazahexacosan-26-yl)oxy)-4-hydroxy-5-(2-hydroxyacetamido)tetrahydro-2H-pyran-2-carboxylic acid (264M) as white solid, Yield: 59 mg, 24.93%; LCMS (ESI+) m/z 974.80 [M+H]⁺. ¹H NMR (400 MHz, MeOD) δ 7.31 (s, 4H), 6.83 (s, 2H), 4.02 (s, 2H), 3.82-3.62 (m, 20H), 3.56-3.50 (m, 7H), 3.40-3.28 (m, 5H), 3.33-3.19 (m, 1H), 2.73 (dd, J=13.5, 1H), 2.49 (m, 2H), 2.22-2.18 (m, 2H), 1.61-1.58 (t, J=14.4 Hz, 1H), 1.59-1.56 (dt, J=12.0 Hz, 4H), 1.36-1.35 (m, 4H).

### (2R,4S,5R,6R)-6-((1R,2R)-3-(2-(4-chlorophenyl)acetamido)-1,2-dihydroxypropyl)-4-hydroxy-5-(2-hydroxyacetamido)-2-((5,21,38-trioxo-1-((3aR,4R,6aS)-2-oxohexahydro-1H-thieno[3,4-d]imidazol-4-yl)-9,12,15,18,25,28,31,34-octaoxa-6,22,37-triazatetratetracontan-44-yl)oxy)tetrahydro-2H-pyran-2-carboxylic acid (Cpd. 264B)

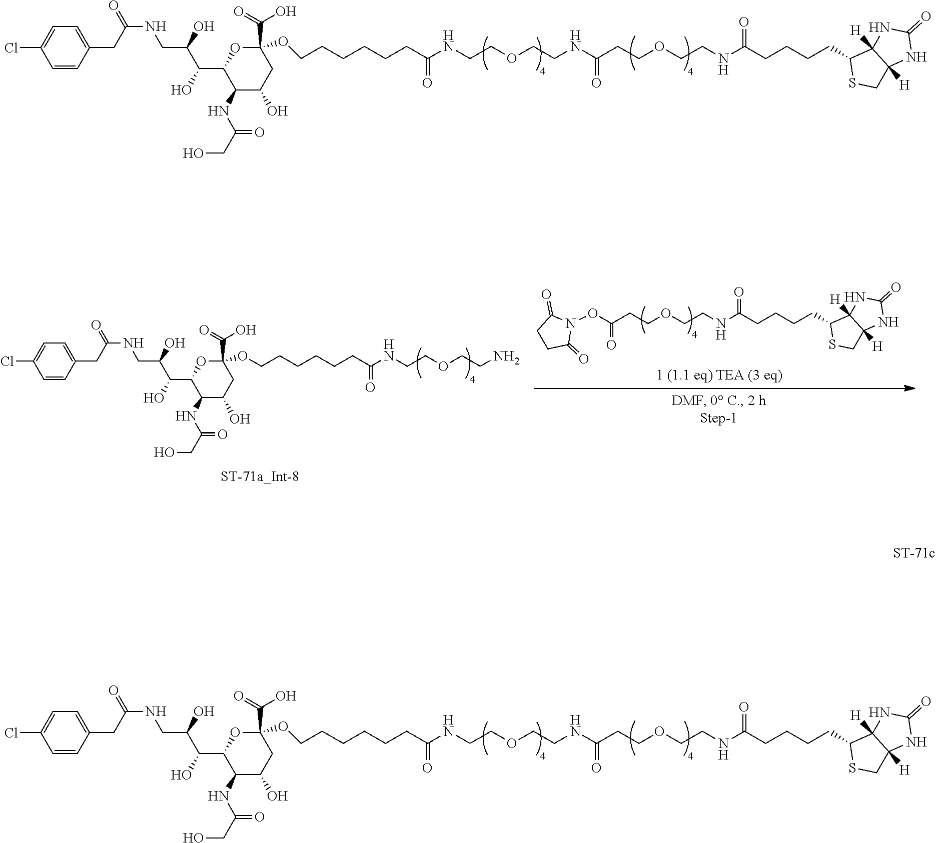

### Synthesis of (2R,4S,5R,6R)-6-((1R,2R)-3-(2-(4-chlorophenyl)acetamido)-1,2-dihydroxypropyl)-4-hydroxy-5-(2-hydroxyacetamido)-2-((5,21,38-trioxo-1-((3aR,4R,6aS)-2-oxohexahydro-1H-thieno[3,4-d]imidazol-4-yl)-9,12,15,18,25,28,31,34-octaoxa-6,22,37-triazatetratetracontan-44-yl)oxy)tetrahydro-2H-pyran-2-carboxylic acid (264B)

To a stirred solution (2R,4S,5R,6R)-2-((1-amino-16-oxo-3,6,9,12-tetraoxa-15-azadocosan-22-yl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (264_Int-8) (50 mg, 0.06 mmol) in N—N-Dimethylformamide (0.5 mL) were added 2,5-dioxopyrrolidin-1-yl 17-oxo-21-((3aR,4R,6aS)-2-oxohexahydro-1H-thieno[3,4-d]imidazol-4-yl)-4,7,10,13-tetraoxa-16-azahenicosanoate (1) (39.3 mg, 0.07 mmol) and triethyl amine (0.05 ml, 0.18 mmol) at 0° C. The resulting reaction mixture was stirred at the same temperature for 45 min. After completion, the reaction mixture was concentrated and purified by prep-HPLC using acetonitrile and water (+0.05 TFA) as eluents to afford the desired product (2R,4S,5R,6R)-6-((1R,2R)-3-(2-(4-chlorophenyl)acetamido)-1,2-dihydroxypropyl)-4-hydroxy-5-(2-hydroxyacetamido)-2-((5,21,38-trioxo-1-((3aR,4R,6aS)-2-oxohexahydro-1H-thieno[3,4-d]imidazol-4-yl)-9,12,15,18,25,28,31,34-octaoxa-6,22,37-triazatetratetracontan-44-yl)oxy)tetrahydro-2H-pyran-2-carboxylic acid (264B) as white solid, Yield: 50 mg, 63.48%; LCMS (ESI−) m/z 1294.8 [M−H]⁻. ¹H NMR (400 MHz, MeOD): δ 7.32 (s, 4H), 4.52-4.49 (m, 1H), 4.34-4.32 (dd, J=8.0 Hz, 1H), 3.89 (m, 1H), 3.76-3.71 (m, 7H), 3.68-3.62 (m, 29H), 3.57-3.57 (m, 7H), 3.39-3.34 (m, 6H), 3.24-3.21 (m, 2H), 2.97-2.92 (dd, J=18 Hz, 1H), 2.74-2.71 (m, 2H), 2.48 (t, J=12.4 Hz, 2H), 2.25 (t, J=14.4 Hz, 2H), 2.0 (s, 3H), 1.73-1.57 (m, 11H), 1.48-1.31 (m, 8H).

### (2R,4S,5R,6R)-6-((1R,2R)-3-(2-(4-chlorophenyl)acetamido)-1,2-dihydroxypropyl)-4-hydroxy-5-(2-hydroxyacetamido)-2-((6-(1-(2-oxo-6,9,12,15-tetraoxa-3-azaheptadecan-17-yl)-1H-1,2,3-triazol-4-yl)hexyl)oxy)tetrahydro-2H-pyran-2-carboxylic acid (Cpd. 263)

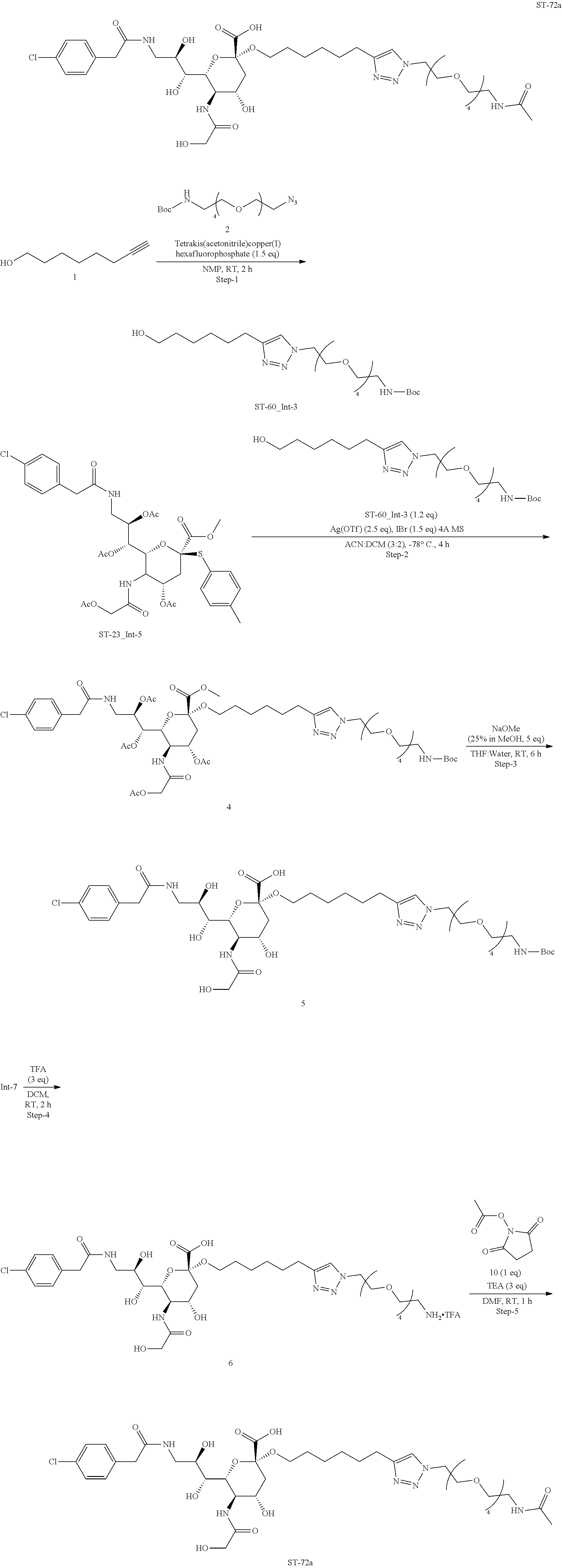

### Synthesis tert-butyl (14-(4-(6-hydroxyhexyl)-1H-1,2,3-triazol-1-yl)-3,6,9,12-tetraoxatetradecyl) carbamate (ST-60_Int-3)

To a stirred solution of oct-7-yn-1-ol (1) (2 g, 15.87 mmol) and tert-butyl (3,6,9,12-tetraoxapentadec-14-yn-1-yl) carbamate (2) (5.74 g, 15.87 mmol.) in NMP (20 mL) was added Tetrakis(acetonitrile)copper(I)hexafluorophosphate (9.7 g, 26.3 mmol, 1.5 eq) and the reaction mixture was stirred at room temperature for 2 h. After completion, the reaction mixture was concentrated under reduced pressure and the crude residue obtained was purified by C18 reverse phase column chromatography using water and acetonitrile (+0.05% TFA) as eluents to get the desired compound (ST-60_Int-3) as colourless solid, Yield: 4.1 g, 52%. ¹HNMR-δ 7.80 (s, 1H), 4.46 (t, J=5.3 Hz, 2H), 3.80 (t, J=5.3 Hz, 2H), 3.56-3.45 (m, 16H), 3.38 (td, J=6.3, 2.5 Hz, 7H), 3.06 (q, J=6.0 Hz, 2H), 2.60 (t, J=7.6 Hz, 2H), 1.57 (td, J=11.2, 6.6 Hz, 2H), 1.41 (t, J=6.8 Hz, 3H), 1.38 (s, 8H), 1.31 (td, J=5.9, 3.3 Hz, 5H).

### Synthesis of (1R,2R)-1-((2R,3R,4S,6R)-4-acetoxy-3-(2-acetoxyacetamido)-6-((6-(1-(2,2-dimethyl-4-oxo-3,8,11,14,17-pentaoxa-5-azanonadecan-19-yl)-1H-1,2,3-triazol-4-yl)hexyl)oxy)-6-(methoxycarbonyl)tetrahydro-2H-pyran-2-yl)-3-(2-(4-chlorophenyl)acetamido)propane-1,2-diyl diacetate (4)

To a stirred solution of ((1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (ST-23_Int-5) (2 g, 2.61 mmol), tert-butyl (14-(4-(6-hydroxyhexyl)-1H-1,2,3-triazol-1-yl)-3,6,9,12-tetraoxatetradecyl)carbamate (ST-60_Int-3) (1.53 g, 3.13 mmol), Silver (1+) trifluoro methane sulfonate (AgOTf) (1.67 g, 6.53 mmol) in anhydrous dichloromethane (24 mL) and anhydrous acetonitrile (36 mL) was added activated 4 Å powdered molecular sieves (5 g). The reaction mixture was stirred at room temperature for 1 h under nitrogen atmosphere. Then cooled to −78° C., iodine monobromide (807 mg, 3.92 mmol) dissolved in dichloromethane (3 mL) was added dropwise and the resulting mixture was stirred at same temperature for 2 h. After completion, the reaction mixture was quenched by addition of triethylamine (2 mL) at the same temperature and warmed to room temperature. The reaction mixture was then filtered, the filtrate was washed with 10% aqueous sodium thiosulfate solution, dried over sodium sulfate, filtered, concentrated under reduced pressure. The crude residue obtained was purified by C18 reverse phase column chromatography using water and acetonitrile (+0.05% TFA) as eluents to afford the desired product (1R,2R)-1-((2R,3R,4S,6R)-4-acetoxy-3-(2-acetoxyacetamido)-6-((6-(1-(2,2-dimethyl-4-oxo-3,8,11,14,17-pentaoxa-5-azanonadecan-19-yl)-1H-1,2,3-triazol-4-yl)hexyl)oxy)-6-(methoxycarbonyl)tetrahydro-2H-pyran-2-yl)-3-(2-(4-chlorophenyl)acetamido)propane-1,2-diyl diacetate (4) as a pale yellow solid, Yield- 1.2 g, 51.40%; LCMS (ESI) m/z 1123.6 [M+H]⁺.

### Synthesis of (2R,4S,5R,6R)-6-((1R,2R)-3-(2-(4-chlorophenyl)acetamido)-1,2-dihydroxypropyl)-2-((6-(1-(2,2-dimethyl-4-oxo-3,8,11,14,17-pentaoxa-5-azanonadecan-19-yl)-1H-1,2,3-triazol-4-yl)hexyl)oxy)-4-hydroxy-5-(2-hydroxyacetamido)tetrahydro-2H-pyran-2-carboxylic acid (5)

To a stirred solution of (1R,2R)-1-((2R,3R,4S,6R)-4-acetoxy-3-(2-acetoxyacetamido)-6-((6-(1-(2,2-dimethyl-4-oxo-3,8,11,14,17-pentaoxa-5-azanonadecan-19-yl)-1H-1,2,3-triazol-4-yl)hexyl)oxy)-6-(methoxycarbonyl)tetrahydro-2H-pyran-2-yl)-3-(2-(4-chlorophenyl)acetamido)propane-1,2-diyl diacetate (4) (1.1 g, 9.73 mmol) in methanol (9 mL) and water (1 mL) was added Sodium methoxide (25% solution in methanol) (0.6 mL, 2.92 mmol, 6 eq.) in at 0° C. and the reaction mixture was stirred at room temperature for 6 h. After completion, the reaction mixture was treated with acidic resin (Dowex 50, H⁺) to maintain pH˜6 and the suspension was filtered. The filtrate was concentrated under reduced pressure and the crude was purified by C18 reverse phase column chromatography using water and acetonitrile (+0.05% TFA) as eluents to afford the desired product (2R,4S,5R,6R)-6-((1R,2R)-3-(2-(4-chlorophenyl)acetamido)-1,2-dihydroxypropyl)-2-((6-(1-(2,2-dimethyl-4-oxo-3,8,11,14,17-pentaoxa-5-azanonadecan-19-yl)-1H-1,2,3-triazol-4-yl)hexyl)oxy)-4-hydroxy-5-(2-hydroxyacetamido)tetrahydro-2H-pyran-2-carboxylic acid (5) as yellow solid, Yield: 800 mg, 75%; LCMS (ESI) m/z 947.9 [M+H]⁺.

### Synthesis of (2R,4S,5R,6R)-6-((1R,2R)-3-(2-(4-chlorophenyl)acetamido)-1,2-dihydroxypropyl)-4-hydroxy-5-(2-hydroxyacetamido)-2-((6-(1-(1,1,1-trifluoro-2-oxo-6,9,12,15-tetraoxa-314-azaheptadecan-17-yl)-1H-1,2,3-triazol-4-yl)hexyl)oxy)tetrahydro-2H-pyran-2-carboxylicacid (6)

To a stirred solution of ((2R,4S,5R,6R)-6-((1R,2R)-3-(2-(4-chlorophenyl)acetamido)-1,2-dihydroxypropyl)-2-((6-(1-(2,2-dimethyl-4-oxo-3,8,11,14,17-pentaoxa-5-azanonadecan-19-yl)-1H-1,2,3-triazol-4-yl)hexyl)oxy)-4-hydroxy-5-(2-hydroxyacetamido)tetrahydro-2H-pyran-2-carboxylic acid (5) (350 mg, 0.366 mmol) in anhydrous dichloromethane (3.5 mL) was added trifluoro acetic acid (1.05 mL) dropwise at 0° C. and the resulting reaction mixture was stirred at room temperature for 2 h. After completion, the solvent was concentrated and the crude residue obtained was purified by C18 reverse phase column chromatography using water and acetonitrile (+0.05% TFA) as eluents to afford the desired product (2R,4S,5R,6R)-6-((1R,2R)-3-(2-(4-chlorophenyl)acetamido)-1,2-dihydroxypropyl)-4-hydroxy-5-(2-hydroxyacetamido)-2-((6-(1-(1,1,1-trifluoro-2-oxo-6,9,12,15-tetraoxa-314-azaheptadecan-17-yl)-1H-1,2,3-triazol-4-yl)hexyl)oxy)tetrahydro-2H-pyran-2-carboxylic acid (6) as yellow solid, Yield: 250 mg, 71%; LCMS (ESI+) m/z 847.9 [M+H]⁺.

### Synthesis of (2R,4S,5R,6R)-6-((1R,2R)-3-(2-(4-chlorophenyl)acetamido)-1,2-dihydroxypropyl)-4-hydroxy-5-(2-hydroxyacetamido)-2-((6-(1-(2-oxo-6,9,12,15-tetraoxa-3-azaheptadecan-17-yl)-1H-1,2,3-triazol-4-yl)hexyl)oxy)tetrahydro-2H-pyran-2-carboxylic acid (263)

To a stirred solution of (2R,4S,5R,6R)-6-((1R,2R)-3-(2-(4-chlorophenyl)acetamido)-1,2-dihydroxypropyl)-4-hydroxy-5-(2-hydroxyacetamido)-2-((6-(1-(1,1,1-trifluoro-2-oxo-6,9,12,15-tetraoxa-314-azaheptadecan-17-yl)-1H-1,2,3-triazol-4-yl)hexyl)oxy)tetrahydro-2H-pyran-2-carboxylic acid (6) (70 mg, 0.08 mmol) in DMF (1 mL) was added 2,5-dioxopyrrolidin-1-yl acetate (11) (12 mg, 0.08 mmol) at 0° C. The resulting reaction mixture was stirred at the same temperature for 1 h. After completion, the reaction mixture was purified by C18 reverse phase column chromatography using water and acetonitrile (+0.05% TFA) as eluents to afford the desired product (2R,4S,5R,6R)-6-((1R,2R)-3-(2-(4-chlorophenyl)acetamido)-1,2-dihydroxypropyl)-4-hydroxy-5-(2-hydroxyacetamido)-2-((6-(1-(2-oxo-6,9,12,15-tetraoxa-3-azaheptadecan-17-yl)-1H-1,2,3-triazol-4-yl)hexyl)oxy)tetrahydro-2H-pyran-2-carboxylic acid (263) as white solid, Yield:35 mg, 54%; LCMS (ESI+) m/z 887.6 [M+H]⁺. ¹H NMR (401 MHz, MeOD) δ 1H NMR (401 MHz, MeOD) δ 7.31 (s, 4H), 4.58 (t, J=5.0 Hz, 2H), 4.04 (s, 2H), 3.91 (t, J=5.2 Hz, 3H), 3.79 (dd, J=20.1, 9.3 Hz, 3H), 3.71 (d, J=9.9 Hz, 2H), 3.63 (dt, J=4.3, 2.2 Hz, 10H), 3.54 (d, J=6.2 Hz, 4H), 2.72 (t, 3H), 1.95 (d, J=1.9 Hz, 3H), 1.77-1.53 (m, 6H), 1.40 (s, 3H).

### (2R,4S,5R,6R)-6-((1R,2R)-3-(2-(4-chlorophenyl)acetamido)-1,2-dihydroxypropyl)-2-((6-(1-(18-(2,5-dioxo-2,5-dihydro-1H-pyrrol-1-yl)-16-oxo-3,6,9,12-tetraoxa-15-azaoctadecyl)-1H-1,2,3-triazol-4-yl)hexyl)oxy)-4-hydroxy-5-(2-hydroxyacetamido)tetrahydro-2H-pyran-2-carboxylic acid (Cpd. 263M)

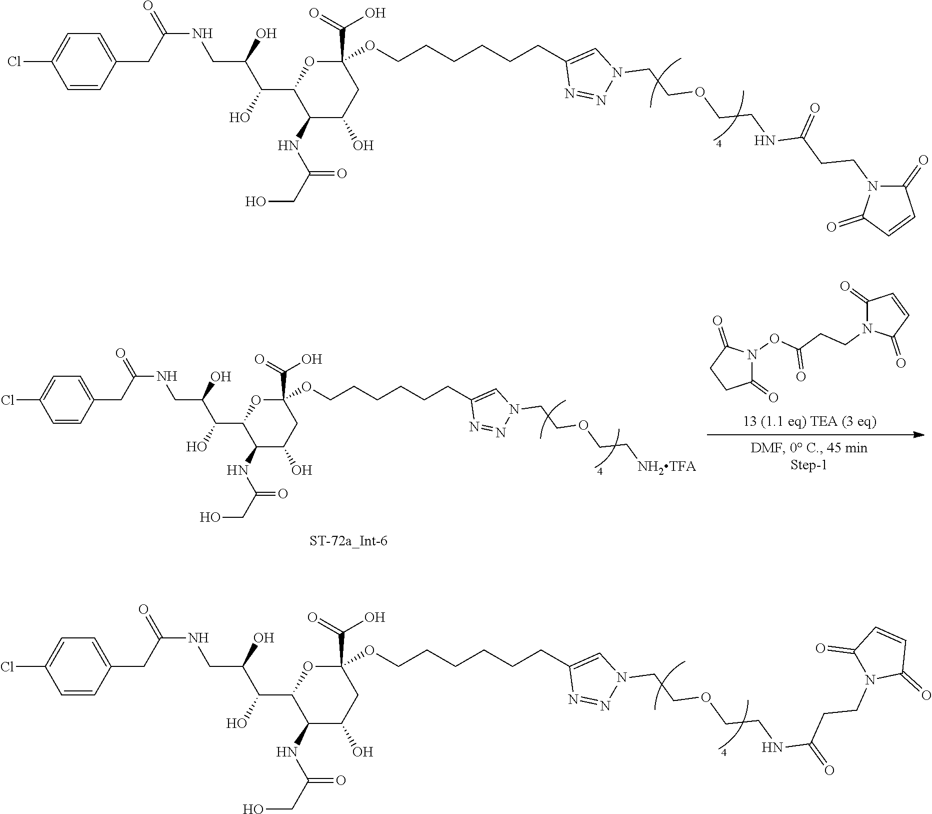

### Synthesis of (2R,4S,5R,6R)-6-((1R,2R)-3-(2-(4-chlorophenyl)acetamido)-1,2-dihydroxypropyl)-2-((6-(1-(18-(2,5-dioxo-2,5-dihydro-1H-pyrrol-1-yl)-16-oxo-3,6,9,12-tetraoxa-15-azaoctadecyl)-1H-1,2,3-triazol-4-yl)hexyl)oxy)-4-hydroxy-5-(2-hydroxyacetamido)tetrahydro-2H-pyran-2-carboxylic acid (263M)

To a stirred solution of (2R,4S,5R,6R)-6-((1R,2R)-3-(2-(4-chlorophenyl)acetamido)-1,2-dihydroxypropyl)-4-hydroxy-5-(2-hydroxyacetamido)-2-((6-(1-(1,1,1-trifluoro-2-oxo-6,9,12,15-tetraoxa-314-azaheptadecan-17-yl)-1H-1,2,3-triazol-4-yl)hexyl)oxy)tetrahydro-2H-pyran-2-carboxylic acid (263_Int-6) (150 mg, 0.16 mmol) in N,N-dimethyl formamide (2 mL) were added 2,5-dioxopyrrolidin-1-yl 3-(2,5-dioxo-2,5-dihydro-1H-pyrrol-1-yl)propanoate (1) (46 mg, 0.174 mmol) and triethylamine (0.01 mL, 0.47 mmol) at 0° C. The resulting reaction mixture was stirred at the same temperature for 45 min. After completion, the reaction mixture was concentrated and purified by prep-HPLC purification by using acetonitrile and water (+0.05%, TFA) as eluents to (2R,4S,5R,6R)-6-((1R,2R)-3-(2-(4-chlorophenyl)acetamido)-1,2-dihydroxypropyl)-2-((6-(1-(18-(2,5-dioxo-2,5-dihydro-1H-pyrrol-1-yl)-16-oxo-3,6,9,12-tetraoxa-15-azaoctadecyl)-1H-1,2,3-triazol-4-yl)hexyl)oxy)-4-hydroxy-5-(2-hydroxyacetamido)tetrahydro-2H-pyran-2-carboxylic acid (263M) as white solid, Yield: 59 mg, 24.93%; LCMS (ESI+) m/z 974.80 [M+H]⁺. ¹H NMR (400 MHz, MeOD) δ 7.31 (s, 4H), 6.83 (s, 2H), 4.05 (s, 2H), 3.94-3.87 (m, 3H), 3.78 (t, J=6.9 Hz, 4H), 3.71 (dd, J=8.9, 2.5 Hz, 2H), 3.61 (dq, J=5.5, 2.8 Hz, 14H), 3.56-3.46 (m, 6H), 3.39-3.28 (m, 8H), 3.20 (dd, J=13.6, 8.1 Hz, 1H), 2.84 (dd, J=12.4, 4.5 Hz, 1H), 2.70 (t, J=7.7 Hz, 2H), 2.48 (t, J=6.9 Hz, 2H), 1.72-1.64 (m, 2H), 1.56 (dd, J=15.1, 9.1 Hz, 3H), 1.43-1.32 (m, 5H).

### (2R,4S,5R,6R)-6-((1R,2R)-3-(2-(4-chlorophenyl)acetamido)-1,2-dihydroxypropyl)-2-((6-(1-(16,32-dioxo-36-((3aR,4R,6aS)-2-oxohexahydro-1H-thieno[3,4-d]imidazol-4-yl)-3,6,9,12,19,22,25,28-octaoxa-15,31-diazahexatriacontyl)-1H-1,2,3-triazol-4-yl)hexyl)oxy)-4-hydroxy-5-(2-hydroxyacetamido)tetrahydro-2H-pyran-2-carboxylic acid (Cpd. 263B)

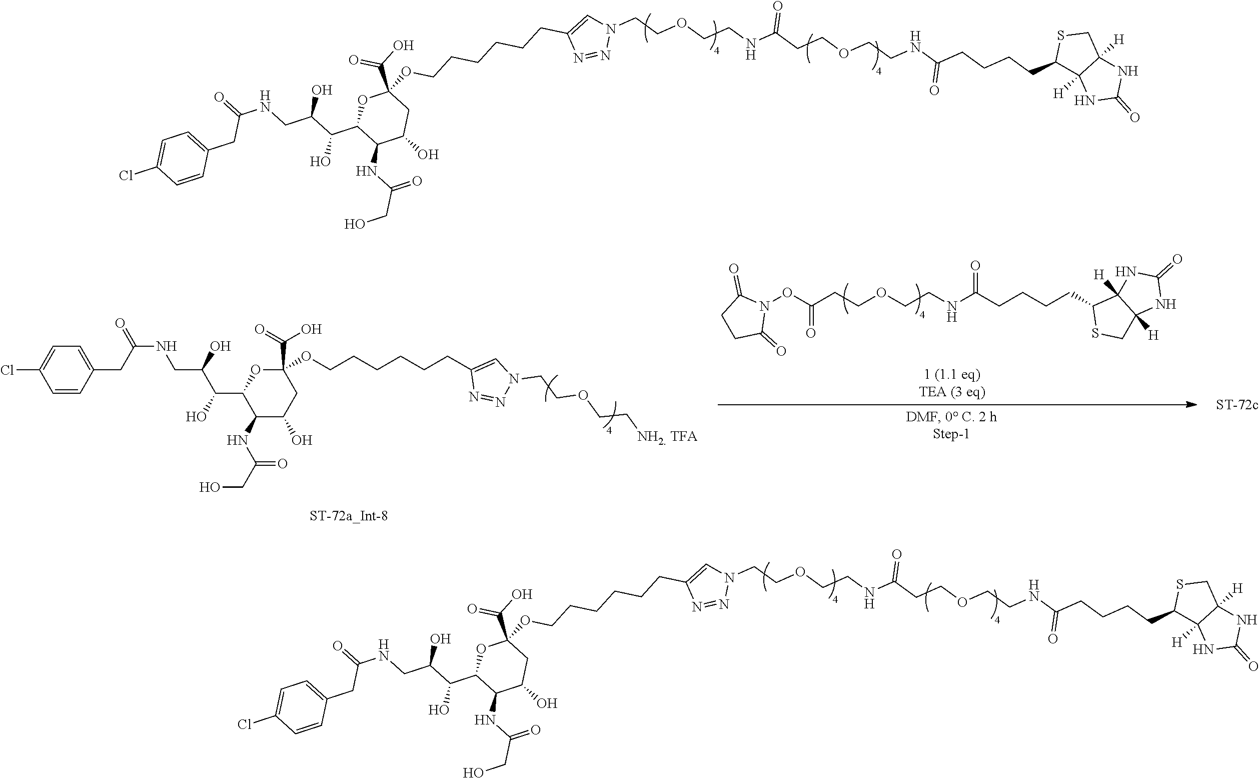

### Synthesis of (2R,4S,5R,6R)-6-((1R,2R)-3-(2-(4-chlorophenyl)acetamido)-1,2-dihydroxypropyl)-2-((6-(1-(16,32-dioxo-36-((3aR,4R,6aS)-2-oxohexahydro-1H-thieno[3,4-d]imidazol-4-yl)-3,6,9,12,19,22,25,28-octaoxa-15,31-diazahexatriacontyl)-1H-1,2,3-triazol-4-yl)hexyl)oxy)-4-hydroxy-5-(2-hydroxyacetamido)tetrahydro-2H-pyran-2-carboxylic acid (264B)

To a stirred solution (2R,4S,5R,6R)-6-((1R,2R)-3-(2-(4-chlorophenyl)acetamido)-1,2-dihydroxypropyl)-4-hydroxy-5-(2-hydroxyacetamido)-2-((6-(1-(1,1,1-trifluoro-2-oxo-6,9,12,15-tetraoxa-314-azaheptadecan-17-yl)-1H-1,2,3-triazol-4-yl)hexyl)oxy)tetrahydro-2H-pyran-2-carboxylic acid (263_Int-6) (50 mg, 0.05 mmol) in N—N-Dimethylformamide (0.5 mL) were added 2,5-dioxopyrrolidin-1-yl 17-oxo-21-((3aR,4R,6aS)-2-oxohexahydro-1H-thieno[3,4-d]imidazol-4-yl)-4,7,10,13-tetraoxa-16-azahenicosanoate (1) (34.2 mg, 0.05 mmol) and triethyl amine (0.05 ml, 0.18 mmol) at 0° C. The resulting reaction mixture was stirred at the same temperature for 45 min. After completion, the reaction mixture was concentrated and purified by prep-HPLC purification by using acetonitrile and water (+0.05 TFA) as eluents to afford the desired product (2R,4S,5R,6R)-6-((1R,2R)-3-(2-(4-chlorophenyl)acetamido)-1,2-dihydroxypropyl)-2-((6-(1-(16,32-dioxo-36-((3aR,4R,6aS)-2-oxohexahydro-1H-thieno[3,4-d]imidazol-4-yl)-3,6,9,12,19,22,25,28-octaoxa-15,31-diazahexatriacontyl)-1H-1,2,3-triazol-4-yl)hexyl)oxy)-4-hydroxy-5-(2-hydroxyacetamido)tetrahydro-2H-pyran-2-carboxylic acid (263B) as white solid, Yield: 50 mg, 63.48%; LCMS (ESI+) m/z 1294.8 [M−H]⁻. ¹H NMR (400 MHz, MeOD): δ 7.84 (d, J=11.2 Hz, 1H), 7.31 (s, 4H), 4.61-4.48 (m, 3H), 4.04 (s, 2H), 3.94-3.87 (m, 3H), 3.86-3.71 (m, 6H), 3.68-3.62 (m, 22H), 3.57-3.52 (m, 6H), 3.39 (ddd, J=8.0, 6.5, 3.1 Hz, 6H), 3.28-3.22 (m, 2H), 2.92 (dd, J=18 Hz, 1H), 2.77-2.68 (m, 4H), 2.47 (t, J=6.2 Hz, 2H), 2.24 (t, J=7.4 Hz, 2H), 1.81-1.55 (m, 10H), 1.51-1.34 (m, 7H).

### (2R,4S,5R,6R)-6-((1R,2R)-3-(2-(4-chlorophenyl)acetamido)-1,2-dihydroxypropyl)-4-hydroxy-5-(2-hydroxyacetamido)-2-((6-((1-(2-oxo-6,9,12,15-tetraoxa-3-azaheptadecan-17-yl)-1H-1,2,3-triazol-4-yl)methoxy)hexyl)oxy)tetrahydro-2H-pyran-2-carboxylic acid (Cpd. 262)

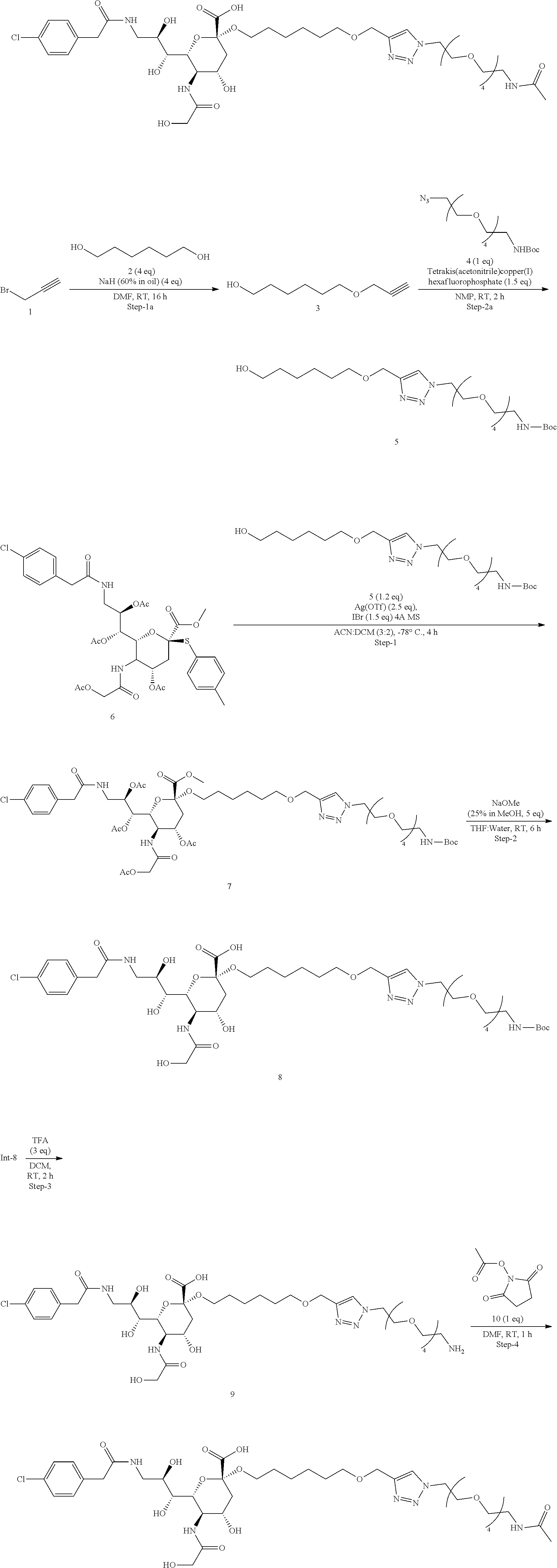

### Synthesis of 6-(prop-2-yn-1-yloxy) hexan-1-ol (3)

To a stirred solution of 3-bromoprop-1-yne (1) (10 g, 84.06 mmol) and hexane-1,6-diol (2) (19.86 g, 168.12 mmol) in DMF (100 mL) was added Sodium hydride (60% in oil) (13.45 g, 336.16 mmol) at 0° C. then reaction mixture was stirred at room temperature for 16 h. After completion, the reaction mixture was quenched by cold water, compound was extracted by Ethyl acetate, dried over sodium sulfate, filtered, concentrated under reduced pressure then purified by column chromatography using ethyl acetate and hexane as eluents to afford 6-(prop-2-yn-1-yloxy) hexan-1-ol (3) as off-white gummy liquid, Yield: 7 g, 53.30%. ¹H NMR (400 MHz, CDCL3); δ 4.13 (t, 2H), 4.06 (t, 1H), 3.63 (t, 2H), 3.52 (t, 2H), 2.44 (t, 1H), 1.66-1.54 (m, 4H), 1.43-1.28 (m, 4H).

### Synthesis of tert-butyl (14-(4-(((6-hydroxyhexyl)oxy) methyl)-1H-1,2,3-triazol-1-yl)-3,6,9,12-tetraoxatetradecyl) carbamate (5)

To a stirred solution of 6-(prop-2-yn-1-yloxy)hexan-1-ol (3) (3 g, 19.57 mmol) and tert-butyl (14-azido-3,6,9,12-tetraoxatetradecyl)carbamate (4) (7.09 g, 19.57 mmol.) in NMP (30 mL) was added Tetrakis(acetonitrile)copper(I)hexafluorophosphate (10.94 g, 29.35 mmol) and the reaction mixture was stirred at room temperature for 2 h. After completion, the reaction mixture was concentrated under reduced pressure to get the crude material, which was purified by C18 reverse phase chromatography using acetonitrile and water (+0.05% TFA) as eluents to afford the desired compound tert-butyl (14-(4-(((6-hydroxyhexyl)oxy)methyl)-1H-1,2,3-triazol-1-yl)-3,6,9,12-tetraoxatetradecyl)carbamate (5) as colorless solid, Yield: 3.1 g, 31.13%. LCMS (ESI+) m/z 519.6 [M+H]⁺.

### Synthesis of (1R,2R)-1-((2R,3R,4S,6R)-4-acetoxy-3-(2-acetoxyacetamido)-6-((6-((1-(2,2-dimethyl-4-oxo-3,8,11,14,17-pentaoxa-5-azanonadecan-19-yl)-1H-1,2,3-triazol-4-yl)methoxy)hexyl)oxy)-6-(methoxycarbonyl)tetrahydro-2H-pyran-2-yl)-3-(2-(4-chlorophenyl)acetamido)propane-1,2-diyl diacetate (7)

To a stirred solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-chlorophenyl)acetamido)propane-1,2-diyl diacetate (6) (2 g, 2.61 mmol), tert-butyl (14-(4-(((6-hydroxyhexyl)oxy)methyl)-1H-1,2,3-triazol-1-yl)-3,6,9,12-tetraoxatetradecyl)carbamate (5) (1.63 g, 3.13 mmol) and Silver (1+) trifluoro methane sulfonate (AgOTf) (1.67 g, 6.53 mmol) in anhydrous dichloromethane (12 mL) and anhydrous acetonitrile (24 mL) was added activated 4 Å powdered molecular sieves (5 g) and the reaction mixture was stirred at room temperature for 1 h under nitrogen. Then the solution was cooled to −78° C., iodine monobromide (0.81 g, 3.92 mmol) dissolved in dichloromethane (4 mL) was added dropwise, and the resulting mixture was stirred at the same temperature for 2 h. After completion, the reaction mixture was quenched by addition of triethylamine (5 mL) and allowed to stir at room temperature for 1 h. Then the reaction mixture was filtered, the filtrate was washed with aqueous sodium thiosulfate solution, dried over sodium sulfate, filtered, and concentrated under reduced pressure. The crude residue obtained was purified by C18 reverse phase column chromatography using water and acetonitrile (+0.05% TFA) as eluents to afford the desired product (1R,2R)-1-((2R,3R,4S,6R)-4-acetoxy-3-(2-acetoxyacetamido)-6-((6-((1-(2,2-dimethyl-4-oxo-3,8,11,14,17-pentaoxa-5-azanonadecan-19-yl)-1H-1,2,3-triazol-4-yl)methoxy)hexyl)oxy)-6-(methoxycarbonyl)tetrahydro-2H-pyran-2-yl)-3-(2-(4-chlorophenyl)acetamido)propane-1,2-diyl diacetate (7) as off-white solid, Yield: 1.2 g, 39.59%; LCMS (ESI±) m/z 1160.6 [M+H]⁺.

### Synthesis of (2R,4S,5R,6R)-6-((1R,2R)-3-(2-(4-chlorophenyl)acetamido)-1,2-dihydroxypropyl)-2-((6-((1-(2,2-dimethyl-4-oxo-3,8,11,14,17-pentaoxa-5-azanonadecan-19-yl)-1H-1,2,3-triazol-4-yl)methoxy)hexyl)oxy)-4-hydroxy-5-(2-hydroxyacetamido)tetrahydro-2H-pyran-2-carboxylic acid (8)

To a stirred solution of (1R,2R)-1-((2R,3R,4S,6R)-4-acetoxy-3-(2-acetoxyacetamido)-6-((6-((1-(2,2-dimethyl-4-oxo-3,8,11,14,17-pentaoxa-5-azanonadecan-19-yl)-1H-1,2,3-triazol-4-yl)methoxy)hexyl)oxy)-6-(methoxycarbonyl)tetrahydro-2H-pyran-2-yl)-3-(2-(4-chlorophenyl)acetamido)propane-1,2-diyl diacetate (7) (1.2 g, 1.03 mmol) in methanol (10 mL) and water (3 mL) was added NaOMe (25% in MeOH) (0.30 mL, 5.17 mmol) dropwise at 0° c. The resulting reaction mixture was stirred at room temperature for 16 h. After completion, the reaction mixture was treated with acidic resin (Dowex 50, H⁺) to maintain pH˜6 and the suspension was filtered. The filtrate was concentrated under reduced pressure and the crude was purified by C18 reverse phase chromatography using water and acetonitrile (+0.05% TFA) as eluents to afford the desired (2R,4S,5R,6R)-6-((1R,2R)-3-(2-(4-chlorophenyl)acetamido)-1,2-dihydroxypropyl)-2-((6-((1-(2,2-dimethyl-4-oxo-3,8,11,14,17-pentaoxa-5-azanonadecan-19-yl)-1H-1,2,3-triazol-4-yl)methoxy)hexyl)oxy)-4-hydroxy-5-(2-hydroxyacetamido)tetrahydro-2H-pyran-2-carboxylic acid (8) as off-white solid, Yield: 650 mg, 64.26%; LCMS (ESI±) m/z 975.5 [M−H]⁻.

### Synthesis of (2R,4S,5R,6R)-2-((6-((1-(14-amino-3,6,9,12-tetraoxatetradecyl)-1H-1,2,3-triazol-4-yl)methoxy)hexyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (9)

To a stirred solution of (2R,4S,5R,6R)-6-((1R,2R)-3-(2-(4-chlorophenyl)acetamido)-1,2-dihydroxypropyl)-2-((6-((1-(2,2-dimethyl-4-oxo-3,8,11,14,17-pentaoxa-5-azanonadecan-19-yl)-1H-1,2,3-triazol-4-yl)methoxy)hexyl)oxy)-4-hydroxy-5-(2-hydroxyacetamido)tetrahydro-2H-pyran-2-carboxylic acid (8) (650 mg, 0.66 mmol) in anhydrous dichloromethane (4 mL) was added trifluoro acetic acid (0.06 mL) dropwise at 0° C. and the resulting reaction mixture was stirred at room temperature for 3 h. After completion, the solvent was concentrated and the crude residue obtained was purified by prep-HPLC using acetonitrile and water, (+0.05% TFA) to afford the desired product (2R,4S,5R,6R)-2-((6-((1-(14-amino-3,6,9,12-tetraoxatetradecyl)-1H-1,2,3-triazol-4-yl)methoxy)hexyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (9) as off-white solid, Yield: 450 mg, 77.13%; LCMS (ESI+) m/z 877.5 [M+H]⁺.

### Synthesis of (2R,4S,5R,6R)-6-((1R,2R)-3-(2-(4-chlorophenyl)acetamido)-1,2-dihydroxypropyl)-4-hydroxy-5-(2-hydroxyacetamido)-2-((6-((1-(2-oxo-6,9,12,15-tetraoxa-3-azaheptadecan-17-yl)-1H-1,2,3-triazol-4-yl)methoxy)hexyl)oxy)tetrahydro-2H-pyran-2-carboxylic acid (262)

To a stirred solution of (2R,4S,5R,6R)-2-((6-((1-(14-amino-3,6,9,12-tetraoxatetradecyl)-1H-1,2,3-triazol-4-yl)methoxy)hexyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (9) (100 mg, 0.11 mmol) in DMF (1 mL) was added 2,5-dioxopyrrolidin-1-yl acetate (9) (17.9 mg, 0.11 mmol) at 0° C. The resulting reaction mixture was stirred at room temperature for 1 h. After completion, the reaction mixture was concentrated and purified by prep-HPLC using acetonitrile and water (+0.05% TFA) as eluents (+0.05%, TFA) to afford the desired product (2R,4S,5R,6R)-6-((1R,2R)-3-(2-(4-chlorophenyl)acetamido)-1,2-dihydroxypropyl)-4-hydroxy-5-(2-hydroxyacetamido)-2-((6-((1-(2-oxo-6,9,12,15-tetraoxa-3-azaheptadecan-17-yl)-1H-1,2,3-triazol-4-yl)methoxy)hexyl)oxy)tetrahydro-2H-pyran-2-carboxylic acid (Cpd. 262) as a white solid, Yield: 75 mg, 71.57%; LCMS (ESI+) m/z 919.6 [M+H]⁺. ¹H NMR (400 MHz, MeOD) δ 8.03 (s, 1H), 7.31 (s, 4H), 4.61-4.59 (t, J=8.8 Hz, 4H), 4.04 (s, 1H), 3.92-4.89 (t, J=10 Hz, 3H), 3.88-3.70 (m, 4H), 3.68 (m, 12H), 3.62 (t, J=6.0 Hz, 6H), 3.55-3.51 (m, 4H), 3.44-3.25 (m, 2H), 2.73 (dd, J=12.8, 4.8 Hz, 1H), 1.95 (s, 1H), 1.59 (t, 1H), 1.59-1.54 (m, 4H), 1.38-1.32 (m, 4H).

### (2R,4S,5R,6R)-6-((1R,2R)-3-(2-(4-chlorophenyl)acetamido)-1,2-dihydroxypropyl)-2-((6-((1-(18-(2,5-dioxo-2,5-dihydro-1H-pyrrol-1-yl)-16-oxo-3,6,9,12-tetraoxa-15-azaoctadecyl)-1H-1,2,3-triazol-4-yl)methoxy)hexyl)oxy)-4-hydroxy-5-(2-hydroxyacetamido)tetrahydro-2H-pyran-2-carboxylic acid (Cpd. 262M)

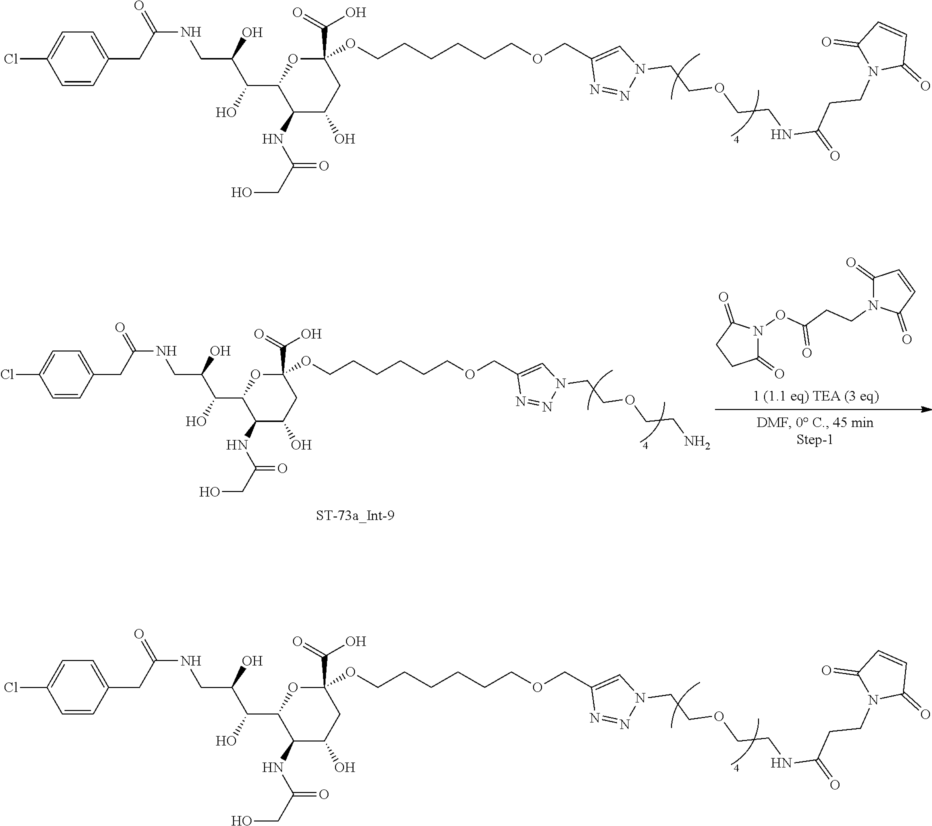

### Synthesis of (2R,4S,5R,6R)-6-((1R,2R)-3-(2-(4-chlorophenyl)acetamido)-1,2-dihydroxypropyl)-2-((6-((1-(18-(2,5-dioxo-2,5-dihydro-1H-pyrrol-1-yl)-16-oxo-3,6,9,12-tetraoxa-15-azaoctadecyl)-1H-1,2,3-triazol-4-yl)methoxy)hexyl)oxy)-4-hydroxy-5-(2-hydroxyacetamido)tetrahydro-2H-pyran-2-carboxylic acid (Cpd. 262M)

To a stirred solution of (2R,4S,5R,6R)-2-((6-((1-(14-amino-3,6,9,12-tetraoxatetradecyl)-1H-1,2,3-triazol-4-yl)methoxy)hexyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (262_nt-9) (120 mg, 0.14 mmol) in N,N-dimethyl formamide (1.2 mL) were added 2,5-dioxopyrrolidin-1-yl 3-(2,5-dioxo-2,5-dihydro-1H-pyrrol-1-yl)propanoate (1) (40.1 mg, 0.15 mmol) and triethylamine (0.06 mL, 0.42 mmol) at 0° C. The resulting reaction mixture was stirred at the same temperature for 45 min. After completion, the reaction mixture was concentrated and purified by prep-HPLC using acetonitrile and water (+0.05%, TFA) as eluents to get desired product (2R,4S,5R,6R)-6-((1R,2R)-3-(2-(4-chlorophenyl)acetamido)-1,2-dihydroxypropyl)-2-((6-((1-(18-(2,5-dioxo-2,5-dihydro-1H-pyrrol-1-yl)-16-oxo-3,6,9,12-tetraoxa-15-azaoctadecyl)-1H-1,2,3-triazol-4-yl)methoxy)hexyl)oxy)-4-hydroxy-5-(2-hydroxyacetamido)tetrahydro-2H-pyran-2-carboxylic acid (Cpd. 262M) as white solid, Yield: 68 mg, 48.34%; LCMS (ESI+) m/z 1028.50 [M+H]⁺. ¹H NMR (400 MHz, MeOD) δ: 8.03 (s, 1H), 7.29 (s, 4H), 6.78 (s, 2H), 4.60-4.58 (t, J=10 Hz, 4H), 4.03 (s, 2H), 3.91-4.89 (t, J=9.6 Hz, 3H), 3.85-3.72 (m, 4H), 3.69-3.61 (m, 14H), 3.53-3.49 (m, 6H), 3.48-3.37 (m, 2H), 3.27-3.23 (m, 2H), 2.74 (dd, J=12.8, 4.8 Hz, 1H), 2.45 (t, 2H), 1.73 (t, 1H), 1.59-1.54 (m, 4H), 1.38-1.32 (m, 4H).

### (2R,4S,5R,6R)-6-((1R,2R)-3-(2-(4-chlorophenyl)acetamido)-1,2-dihydroxypropyl)-2-((6-((1-(16,32-dioxo-36-((3aR,4R,6aS)-2-oxohexahydro-1H-thieno[3,4-d]imidazol-4-yl)-3,6,9,12,19,22,25,28-octaoxa-15,31-diazahexatriacontyl)-1H-1,2,3-triazol-4-yl)methoxy)hexyl)oxy)-4-hydroxy-5-(2-hydroxyacetamido)tetrahydro-2H-pyran-2-carboxylic acid (Cpd. 262B)

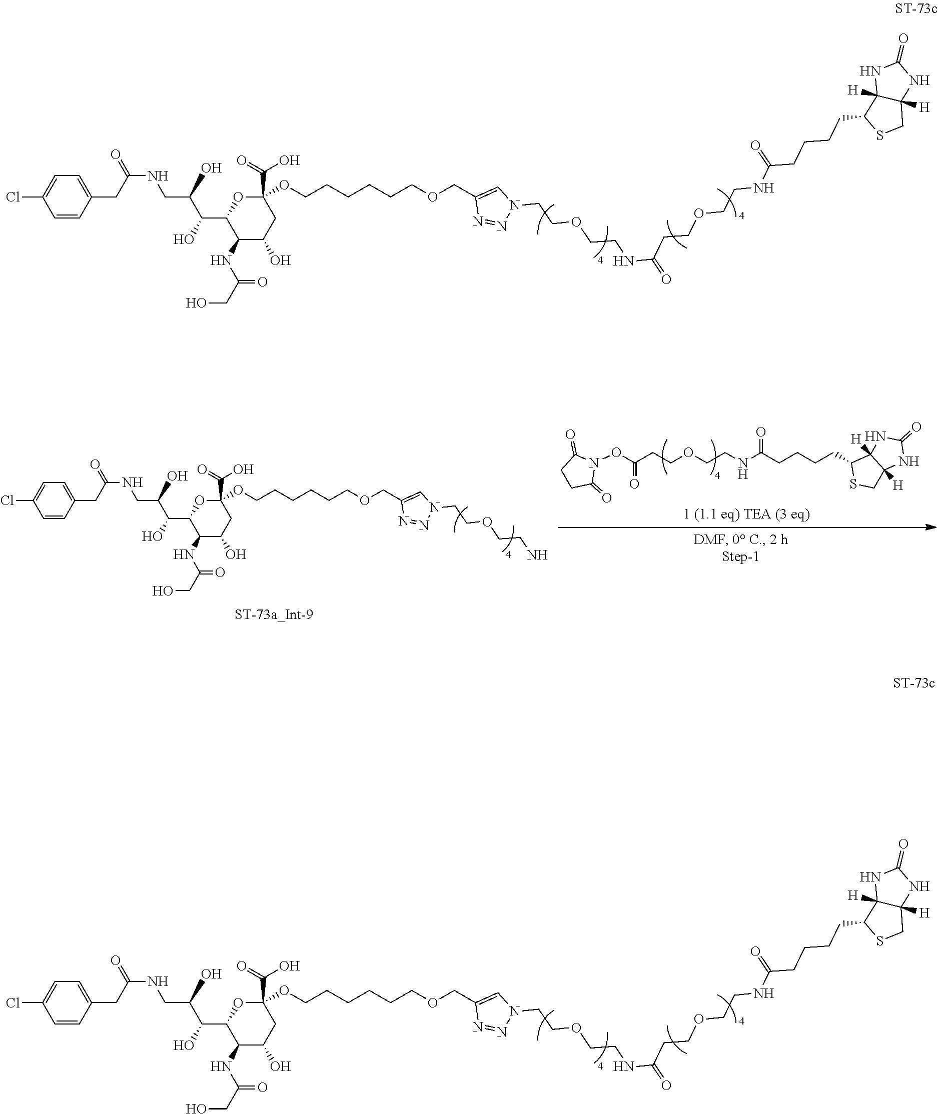

### Synthesis of (2R,4S,5R,6R)-6-((1R,2R)-3-(2-(4-chlorophenyl)acetamido)-1,2-dihydroxypropyl)-2-((6-((1-(16,32-dioxo-36-((3aR,4R,6aS)-2-oxohexahydro-1H-thieno[3,4-d]imidazol-4-yl)-3,6,9,12,19,22,25,28-octaoxa-15,31-diazahexatriacontyl)-1H-1,2,3-triazol-4-yl)methoxy)hexyl)oxy)-4-hydroxy-5-(2-hydroxyacetamido)tetrahydro-2H-pyran-2-carboxylic acid (Cpd. 262B)

To a stirred solution (2R,4S,5R,6R)-2-((6-((1-(14-amino-3,6,9,12-tetraoxatetradecyl)-1H-1,2,3-triazol-4-yl)methoxy)hexyl)oxy)-6-((1R,2R)-3-(2-(4-chlorophenyl)acetamido)-1,2-dihydroxypropyl)-4-hydroxy-5-(2-hydroxyacetamido)tetrahydro-2H-pyran-2-carboxylic acid (262_Int-9) (50 mg, 0.06 mmol) in N—N-Dimethylformamide (0.5 mL) were added 2,5-dioxopyrrolidin-1-yl 17-oxo-21-((3aR,4R,6aS)-2-oxohexahydro-1H-thieno[3,4-d]imidazol-4-yl)-4,7,10,13-tetraoxa-16-azahenicosanoate (1) (39.3 mg, 0.07 mmol) and triethyl amine (0.05 ml, 0.18 mmol) at 0° C. The resulting reaction mixture was stirred at the same temperature for 45 min. After completion, the reaction mixture was concentrated and purified by prep-HPLC using acetonitrile and water (+0.05% TFA) as eluents to afford (2R,4S,5R,6R)-6-((1R,2R)-3-(2-(4-chlorophenyl)acetamido)-1,2-dihydroxypropyl)-2-((6-((1-(16,32-dioxo-36-((3aR,4R,6aS)-2-oxohexahydro-1H-thieno[3,4-d]imidazol-4-yl)-3,6,9,12,19,22,25,28-octaoxa-15,31-diazahexatriacontyl)-1H-1,2,3-triazol-4-yl)methoxy)hexyl)oxy)-4-hydroxy-5-(2-hydroxyacetamido)tetrahydro-2H-pyran-2-carboxylic acid (Cpd. 262B) as white solid, Yield: 32 mg, 41.56%; LCMS (ESI±) m/z 1348.8 [M−H]−. ¹H NMR (400 MHz, MeOD) δ 8.04 (s, 1H), 7.31 (s, 4H), 4.62-4.59 (t, J=9.6 Hz, 4H), 4.50 (m, 1H), 4.33 (m, 1H), 4.03 (s, 2H), 3.92-4.89 (t, J=13.2 Hz, 3H), 3.83 (m, 2H), 3.77-3.70 (t, 4H), 3.65-3.62 (m, 24H), 3.54-3.51 (m, 8H), 3.33-3.23 (m, 10H), 2.95 (dd, J=12.8, 4.8 Hz, 1H), 2.74 (dd, J=12.8, 4.8 Hz, 2H), 2.47 (t, 2H), 2.24 (t, 2H), 1.69-1.56 (m, 8H), 1.47-1.42 (m, 6H).

### (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((6-((1-(2-oxo-6,9,12,15-tetraoxa-3-azaheptadecan-17-yl)-1H-1,2,3-triazol-4-yl)methoxy)hexyl)oxy)tetrahydro-2H-pyran-2-carboxylic acid (Cpd. ST-65a)

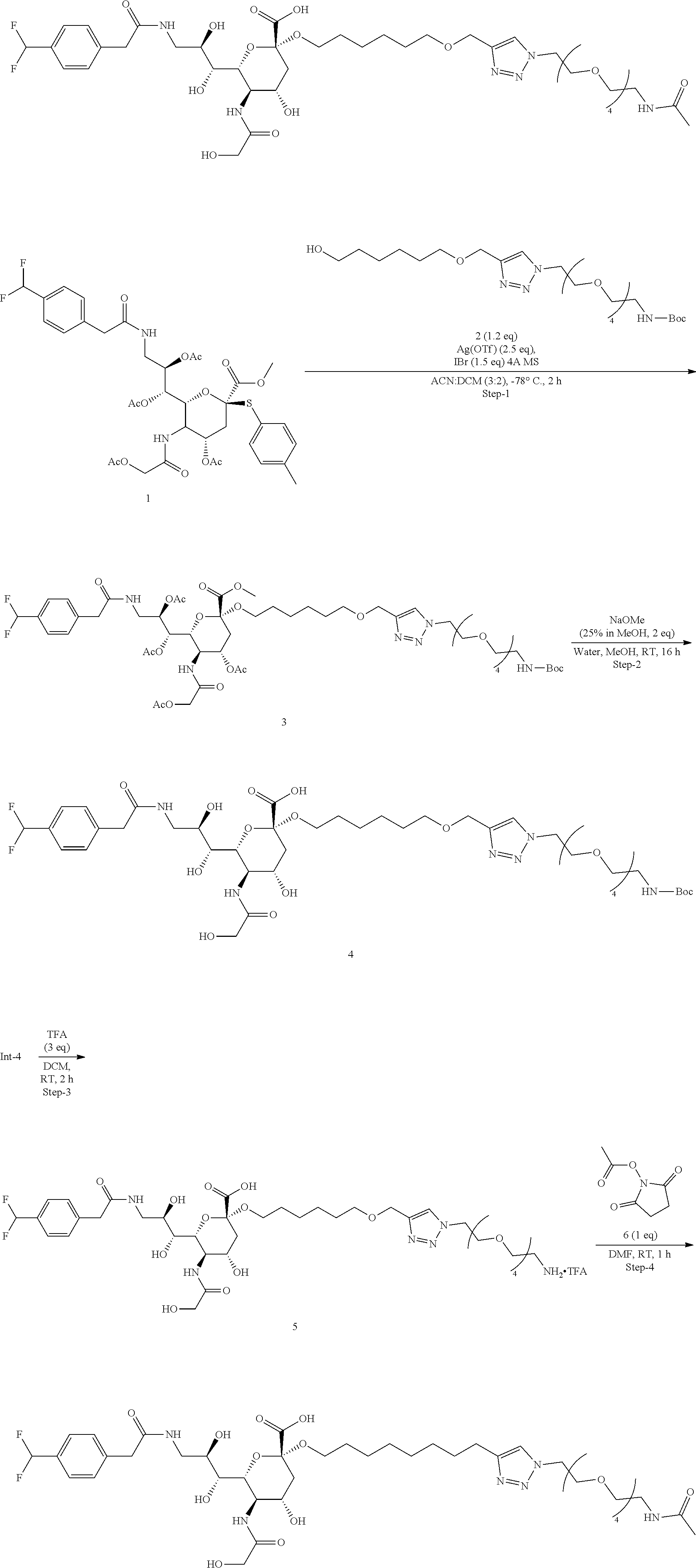

### Synthesis of (1R,2R)-1-((2R,3R,4S,6R)-4-acetoxy-3-(2-acetoxyacetamido)-6-((6-((1-(2,2-dimethyl-4-oxo-3,8,11,14,17-pentaoxa-5-azanonadecan-19-yl)-1H-1,2,3-triazol-4-yl)methoxy)hexyl)oxy)-6-(methoxycarbonyl)tetrahydro-2H-pyran-2-yl)-3-(2-(4-(difluoromethyl)phenyl)acetamido)propane-1,2-diyl diacetate (3)

To a stirred solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-(difluoromethyl)phenyl)acetamido)propane-1,2-diyl diacetate (1) (2 g, 2.56 mmol) and tert-butyl (14-(4-(((6-hydroxyhexyl)oxy)methyl)-1H-1,2,3-triazol-1-yl)-3,6,9,12-tetraoxatetradecyl)carbamate (2) (1.59 g, 3.07 mmol), Silver (1+) trifluoro methane sulfonate (AgOTf) (1.64 g, 6.40 mmol) in anhydrous dichloromethane (12 mL) and anhydrous acetonitrile (24 mL) was added activated 4 Å powdered molecular sieves (5 g) and the reaction mixture was stirred at room temperature for 1 h under nitrogen. The solution was cooled to −78° C., iodine monobromide (794 mg, 3.84 mmol) dissolved in dichloromethane (4 mL) was added dropwise and the resulting mixture was stirred at the same temperature for 2 h. After completion, the reaction mixture was quenched by addition of triethylamine (5 mL). The reaction mixture was filtered, the filtrate was washed with aqueous sodium thiosulfate solution, dried over sodium sulfate, filtered, and concentrated under reduced pressure. The crude residue obtained was purified by C18 reverse phase column chromatography using water: acetonitrile (+0.05% TFA) as eluents to afford the desired product (1R,2R)-1-((2R,3R,4S,6R)-4-acetoxy-3-(2-acetoxyacetamido)-6-((6-((1-(2,2-dimethyl-4-oxo-3,8,11,14,17-pentaoxa-5-azanonadecan-19-yl)-1H-1,2,3-triazol-4-yl)methoxy)hexyl)oxy)-6-(methoxycarbonyl)tetrahydro-2H-pyran-2-yl)-3-(2-(4-(difluoromethyl)phenyl)acetamido)propane-1,2-diyl diacetate (3) as pale yellow solid, Yield: 1.2 g, 39%; LCMS (ESI+) m/z 1176.10 [M+H]⁺.

### Synthesis of (2R,4S,5R,6R)-6-((1R,2R)-3-(2-(4-(difluoromethyl)phenyl)acetamido)-1,2-dihydroxypropyl)-2-((6-((1-(2,2-dimethyl-4-oxo-3,8,11,14,17-pentaoxa-5-azanonadecan-19-yl)-1H-1,2,3-triazol-4-yl)methoxy)hexyl)oxy)-4-hydroxy-5-(2-hydroxyacetamido)tetrahydro-2H-pyran-2-carboxylic acid (4)

To a stirred solution of (1R,2R)-1-((2R,3R,4S,6R)-4-acetoxy-3-(2-acetoxyacetamido)-6-((6-((1-(2,2-dimethyl-4-oxo-3,8,11,14,17-pentaoxa-5-azanonadecan-19-yl)-1H-1,2,3-triazol-4-yl)methoxy)hexyl)oxy)-6-(methoxycarbonyl)tetrahydro-2H-pyran-2-yl)-3-(2-(4-(difluoromethyl)phenyl)acetamido)propane-1,2-diyl diacetate (3) (1.2 g, 1.02 mmol) in methanol (10 mL) and water (3 mL) was added NaOMe (25% in MeOH) (0.42 mL, 1.96 mmol) dropwise at room temperature. The resulting reaction mixture was stirred at room temperature for 16 h. After completion, the reaction mixture was treated with acidic (Dowex 50, H⁺) to maintain pH˜6 and the suspension was filtered. The filtrate was concentrated under reduced pressure and the crude was purified by C18 reverse phase column chromatography using water: acetonitrile (+0.05% TFA) as eluents to afford the desired product (2R,4S,5R,6R)-6-((1R,2R)-3-(2-(4-(difluoromethyl)phenyl)acetamido)-1,2-dihydroxypropyl)-2-((6-((1-(2,2-dimethyl-4-oxo-3,8,11,14,17-pentaoxa-5-azanonadecan-19-yl)-1H-1,2,3-triazol-4-yl)methoxy)hexyl)oxy)-4-hydroxy-5-(2-hydroxyacetamido)tetrahydro-2H-pyran-2-carboxylic acid (4) as an off white solid, Yield: 652 mg, 64%; LCMS (ESI+) m/z 993.90 [M+H]⁺.

### Synthesis of (2R,4S,5R,6R)-2-((6-((1-(14-amino-3,6,9,12-tetraoxatetradecyl)-1H-1,2,3-triazol-4-yl)methoxy)hexyl)oxy)-6-((1R,2R)-3-(2-(4-(difluoromethyl)phenyl)acetamido)-1,2-dihydroxypropyl)-4-hydroxy-5-(2-hydroxyacetamido)tetrahydro-2H-pyran-2-carboxylic acid (5)

To a stirred solution of (2R,4S,5R,6R)-6-((1R,2R)-3-(2-(4-(difluoromethyl)phenyl)acetamido)-1,2-dihydroxypropyl)-2-((6-((1-(2,2-dimethyl-4-oxo-3,8,11,14,17-pentaoxa-5-azanonadecan-19-yl)-1H-1,2,3-triazol-4-yl)methoxy)hexyl)oxy)-4-hydroxy-5-(2-hydroxyacetamido)tetrahydro-2H-pyran-2-carboxylic acid (4) (652 mg, 0.65 mmol) in anhydrous dichloromethane (6.5 mL) was added trifluoro acetic acid (1.9 mL) dropwise at 0° C. and the resulting reaction mixture was stirred at room temperature for 2 h. After completion, the solvent was concentrated to get crude which was purified by prep-HPLC using acetonitrile: water (+0.05% TFA) as eluents to afford the desired product (2R,4S,5R,6R)-2-((6-((1-(14-amino-3,6,9,12-tetraoxatetradecyl)-1H-1,2,3-triazol-4-yl)methoxy)hexyl)oxy)-6-((1R,2R)-3-(2-(4-(difluoromethyl)phenyl)acetamido)-1,2-dihydroxypropyl)-4-hydroxy-5-(2-hydroxyacetamido)tetrahydro-2H-pyran-2-carboxylic acid (5) as a yellow solid, Yield: 320 mg, 62%; LCMS (ESI+) m/z 893.90 [M+H]⁺.

### Synthesis of (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((6-((1-(2-oxo-6,9,12,15-tetraoxa-3-azaheptadecan-17-yl)-1H-1,2,3-triazol-4-yl)methoxy)hexyl)oxy)tetrahydro-2H-pyran-2-carboxylic acid (261)

To a stirred solution of (2R,4S,5R,6R)-2-((6-((1-(14-amino-3,6,9,12-tetraoxatetradecyl)-1H-1,2,3-triazol-4-yl)methoxy)hexyl)oxy)-6-((1R,2R)-3-(2-(4-(difluoromethyl)phenyl)acetamido)-1,2-dihydroxypropyl)-4-hydroxy-5-(2-hydroxyacetamido)tetrahydro-2H-pyran-2-carboxylic acid (5) (120 mg, 0.13 mmol) in DMF (1.2 mL) was added 2,5-dioxopyrrolidin-1-yl acetate (6) (22 mg, 0.14 mmol) at 0° C. The resulting reaction mixture was stirred at 0° C. for 1 h. After completion, the reaction mixture was purified by prep-HPLC purification to afford the desired product (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((6-((1-(2-oxo-6,9,12,15-tetraoxa-3-azaheptadecan-17-yl)-1H-1,2,3-triazol-4-yl)methoxy)hexyl)oxy)tetrahydro-2H-pyran-2-carboxylic acid (261) as a white solid, Yield: 45 mg, 36%; LCMS (ESI+) m/z 935.8 [M+H]⁺. ¹H NMR (400 MHz, MeOD) δ 7.91 (s, 1H), 7.49-7.42 (m, 4H), 6.74 (t, J=56 Hz, 1H), 4.59 (d, J=8.4 Hz, 4H), 4.02 (s, 2H), 3.90-3.61 (m, 22H), 3.54-3.23 (m, 9H), 2.73-2.70 (m, 1H), 1.94 (s, 3H), 1.76-1.70 (m, 1H), 1.59-1.36 (m, 8H).

### (2R,4S,5R,6R)-6-((1R,2R)-3-(2-(4-(difluoromethyl)phenyl)acetamido)-1,2-dihydroxypropyl)-2-((6-((1-(18-(2,5-dioxo-2,5-dihydro-1H-pyrrol-1-yl)-16-oxo-3,6,9,12-tetraoxa-15-azaoctadecyl)-1H-1,2,3-triazol-4-yl)methoxy)hexyl)oxy)-4-hydroxy-5-(2-hydroxyacetamido)tetrahydro-2H-pyran-2-carboxylic acid (Cpd. 261M)

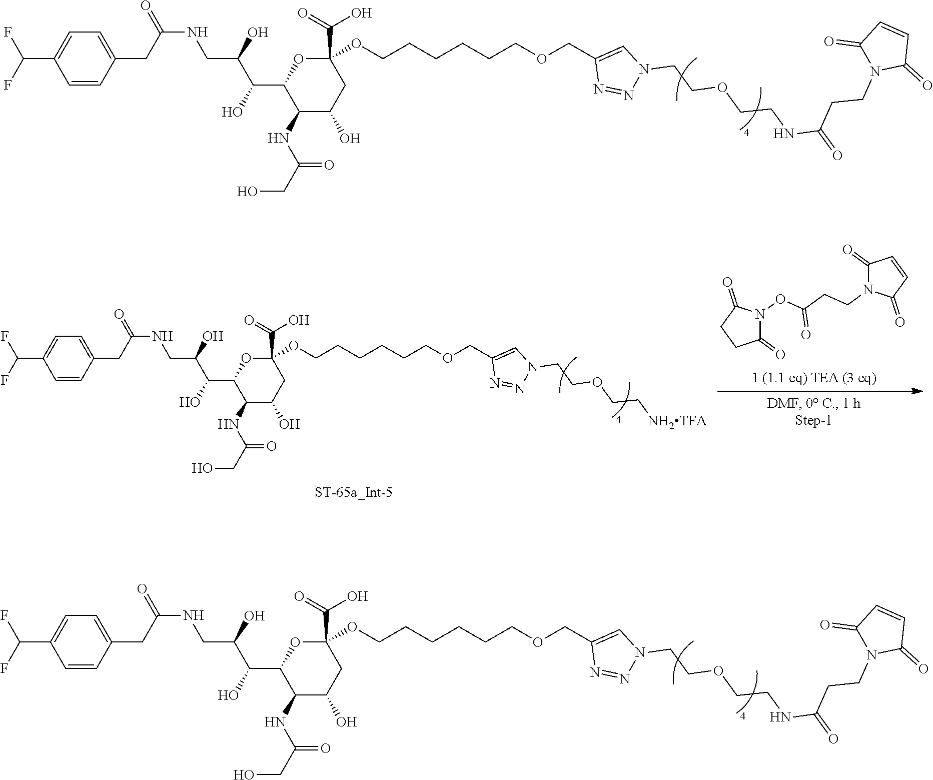

### Synthesis of (2R,4S,5R,6R)-6-((1R,2R)-3-(2-(4-(difluoromethyl)phenyl)acetamido)-1,2-dihydroxypropyl)-2-((6-((1-(18-(2,5-dioxo-2,5-dihydro-1H-pyrrol-1-yl)-16-oxo-3,6,9,12-tetraoxa-15-azaoctadecyl)-1H-1,2,3-triazol-4-yl)methoxy)hexyl)oxy)-4-hydroxy-5-(2-hydroxyacetamido)tetrahydro-2H-pyran-2-carboxylic acid (261M)

To a stirred solution of (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((6-((1-(1,1,1-trifluoro-2-oxo-6,9,12,15-tetraoxa-314-azaheptadecan-17-yl)-1H-1,2,3-triazol-4-yl)methoxy)hexyl)oxy)tetrahydro-2H-pyran-2-carboxylic acid (261_Int-5) (120 mg, 0.12 mmol) in DMF (1.2 mL) were added 2,5-dioxopyrrolidin-1-yl 3-(2,5-dioxo-2,5-dihydro-1H-pyrrol-1-yl)propanoate (1) (35 mg, 0.13 mmol) and triethylamine (0.05 mL, 0.36 mmol) at 0° C. The resulting reaction mixture was stirred at the same temperature for 1 h. After completion, the reaction mixture was purified by prep-HPLC purification to afford the desired product (2R,4S,5R,6R)-6-((1R,2R)-3-(2-(4-(difluoromethyl)phenyl)acetamido)-1,2-dihydroxypropyl)-2-((6-((1-(18-(2,5-dioxo-2,5-dihydro-1H-pyrrol-1-yl)-16-oxo-3,6,9,12-tetraoxa-15-azaoctadecyl)-1H-1,2,3-triazol-4-yl)methoxy)hexyl)oxy)-4-hydroxy-5-(2-hydroxyacetamido)tetrahydro-2H-pyran-2-carboxylic acid (261M) as white solid, Yield: 32 mg, 25%; LCMS (ESI+) m/z 1044.80 [M+H]⁺. ¹H NMR (400 MHz, MeOD) δ 8.02 (s, 1H), 7.45 (dd, J=23, 8 Hz, 4H), 6.82 (s, 2H), 6.74 (t, J=56 Hz, 1H), 4.58 (d, J=4.8 Hz, 4H), 4.02 (s, 2H), 3.91-3.68 (m, 13H), 3.63-3.61 (m, 14H), 3.51-3.25 (m, 8H), 2.75-2.71 (m, 1H), 2.46 (t, J=6.8 Hz, 2H), 1.72-1.30 (m, 9H).

### (2R,4S,5R,6R)-6-((1R,2R)-3-(2-(4-(difluoromethyl)phenyl)acetamido)-1,2-dihydroxypropyl)-2-((6-((1-(16,32-dioxo-36-((3aR,4R,6aS)-2-oxohexahydro-1H-thieno[3,4-d]imidazol-4-yl)-3,6,9,12,19,22,25,28-octaoxa-15,31-diazahexatriacontyl)-1H-1,2,3-triazol-4-yl)methoxy)hexyl)oxy)-4-hydroxy-5-(2-hydroxyacetamido)tetrahydro-2H-pyran-2-carboxylic acid (Cpd. 261B)

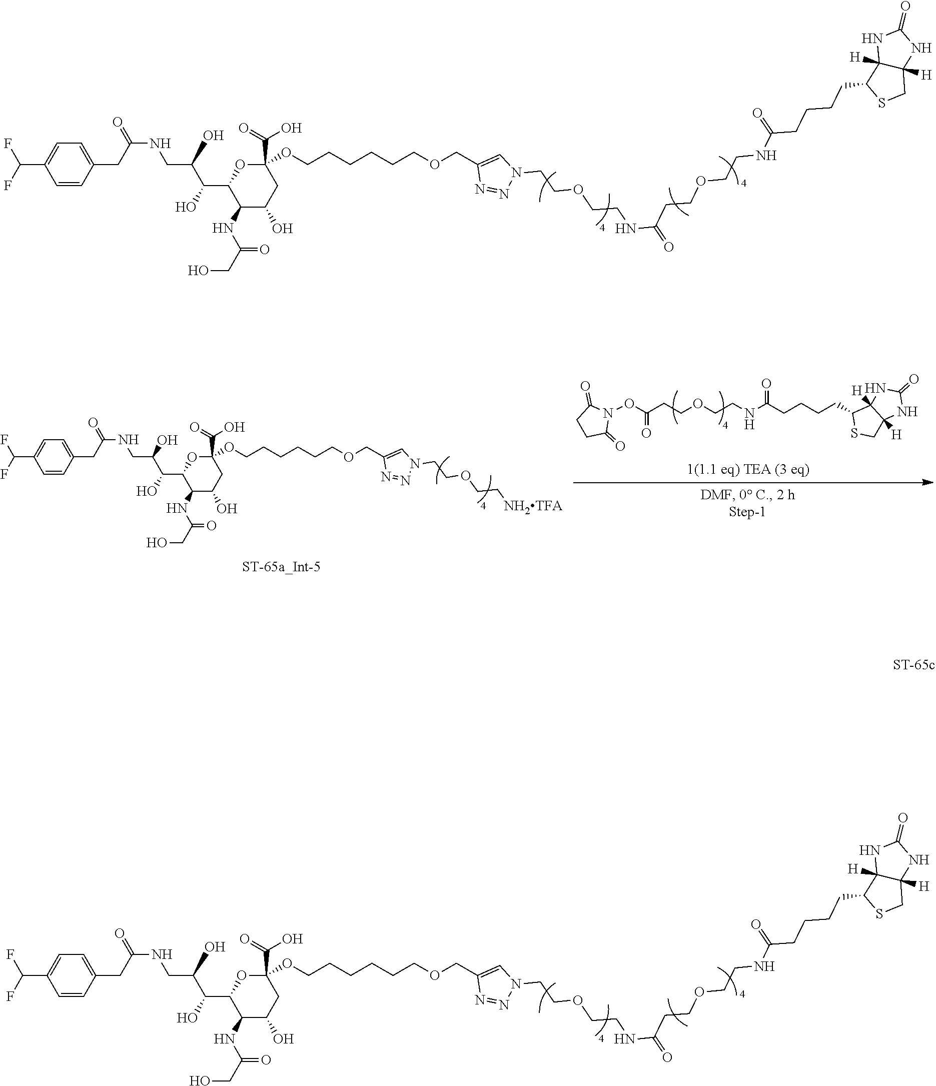

### Synthesis of (2R,4S,5R,6R)-6-((1R,2R)-3-(2-(4-(difluoromethyl)phenyl)acetamido)-1,2-dihydroxypropyl)-2-((6-((1-(16,32-dioxo-36-((3aR,4R,6aS)-2-oxohexahydro-1H-thieno[3,4-d]imidazol-4-yl)-3,6,9,12,19,22,25,28-octaoxa-15,31-diazahexatriacontyl)-1H-1,2,3-triazol-4-yl)methoxy)hexyl)oxy)-4-hydroxy-5-(2-hydroxyacetamido)tetrahydro-2H-pyran-2-carboxylic acid (261B)

To a stirred solution of (2R,4S,5R,6R)-6-((1R,2R)-3-(2-(4-(difluoromethyl)phenyl)acetamido)-1,2-dihydroxypropyl)-4-hydroxy-5-(2-hydroxyacetamido)-2-((6-((1-(1,1,1-trifluoro-2-oxo-6,9,12,15-tetraoxa-314-azaheptadecan-17-yl)-1H-1,2,3-triazol-4-yl)methoxy)hexyl)oxy)tetrahydro-2H-pyran-2-carboxylic acid (261_Int-5) (50 mg, 0.05 mmol) in DMF (0.5 mL) were added 2,5-dioxopyrrolidin-1-yl 3-(2,5-dioxo-2,5-dihydro-1H-pyrrol-1-yl)propanoate (1) (32 mg, 0.06 mmol) and triethylamine (0.02 mL, 0.15 mmol) at 0° C. The resulting reaction mixture was stirred at the same temperature for 2 h. After completion, the reaction mixture was purified by prep-HPLC purification to afford the desired product (2R,4S,5R,6R)-6-((1R,2R)-3-(2-(4-(difluoromethyl)phenyl)acetamido)-1,2-dihydroxypropyl)-2-((6-((1-(16,32-dioxo-36-((3aR,4R,6aS)-2-oxohexahydro-1H-thieno[3,4-d]imidazol-4-yl)-3,6,9,12,19,22,25,28-octaoxa-15,31-diazahexatriacontyl)-1H-1,2,3-triazol-4-yl)methoxy)hexyl)oxy)-4-hydroxy-5-(2-hydroxyacetamido)tetrahydro-2H-pyran-2-carboxylic acid (261B) as white solid, Yield: 25 mg, 37%; LCMS (ESI+) m/z 1367.40 [M+H]⁺. ¹H NMR (400 MHz, MeOD) δ 8.03 (s, 1H), 7.50-7.43 (m, 4H), 6.75 (t, J=56 Hz, 1H), 4.61-4.59 (m, 4H), 4.30-4.35 (m, 1H), 4.03 (s, 2H), 3.92-3.69 (m, 10H), 3.68-3.62 (m, 26H), 3.57-3.25 (m, 14H), 2.98-2.91 (m, 1H), 2.74-2.70 (m, 1H), 2.47 (t, J=6 Hz, 2H), 2.23 (t, J=7.2 Hz, 2H), 1.74-1.37 (m, 15H).

### (2R,4S,5R,6R)-5-acetamido-6-((1R,2R)-3-(3-chlorobenzamido)-1,2-dihydroxypropyl)-2-((2,19-dioxo-6,9,12,15,18-pentaoxa-3,20-diazahexacosan-26-yl) oxy)-4-hydroxytetrahydro-2H-pyran-2-carboxylic acid (Cpd. 268)

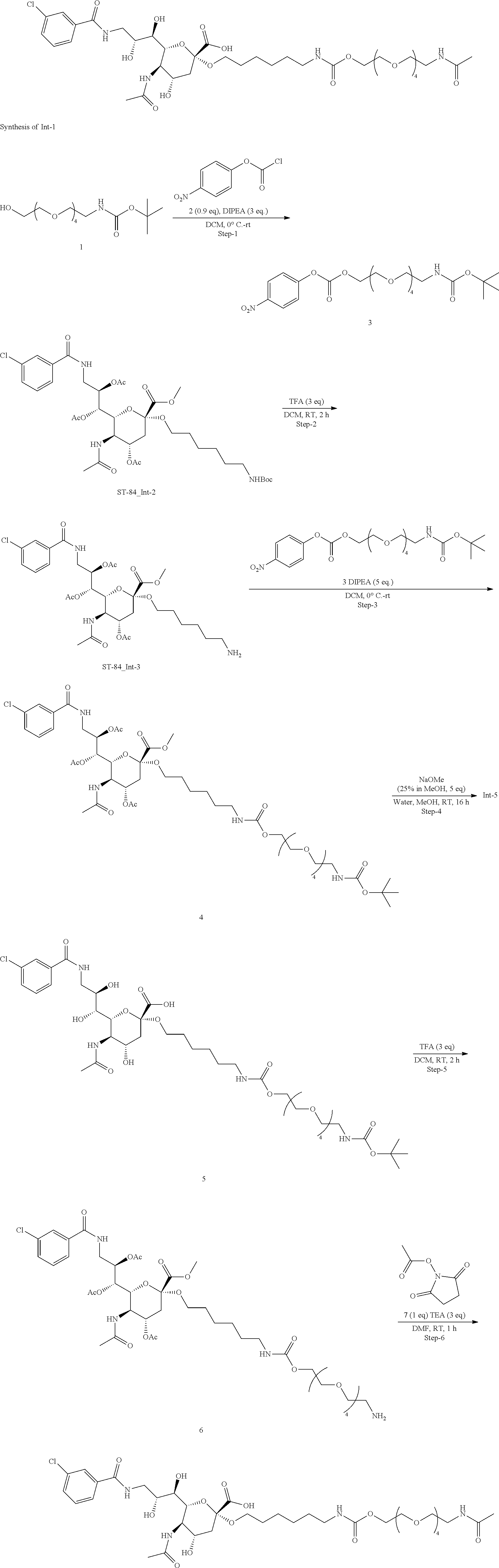

### Synthesis of tert-butyl (2-(2-(((4-nitrophenoxy) carbonyl) oxy) ethoxy)ethyl) carbamate (3)

To a stirred solution of tert-butyl (2-(2-hydroxyethoxy)ethyl)carbamate (1) (500 mg, 2.43 mmol) in anhydrous dichloromethane (5 mL) was added DIPEA (0.7 mL g, 7.31 mmol) and reaction mixture was cooled to 0° C. and 4-nitrophenyl carbonochloridate (2) (377 mg, 2.18 mmol) was added at 0° C. reaction mixture and was stirred at the room temperature for 1 h. After completion, the reaction mixture was concentrated under reduced pressure to get crude as eluents to afford tert-butyl (2-(2-(((4-nitrophenoxy) carbonyl) oxy) ethoxy)ethyl)carbamate (3) as off white solid, Yield: 590 mg.

### Synthesis of (1R,2R)-1-((2R,3R,4S,6R)-3-acetamido-4-acetoxy-6-(methoxycarbonyl)-6-((6-((2,2,2-trifluoroacetyl)-14-azaneyl) hexyl)oxy) tetrahydro-2H-pyran-2-yl)-3-(3-chlorobenzamido) propane-1,2-diyl diacetate (269_Int-3)

To a stirred solution of (1R,2R)-1-((2R,3R,4S,6R)-3-acetamido-4-acetoxy-6-((6-((tert-butoxycarbonyl)amino)hexyl)oxy)-6-(methoxycarbonyl)tetrahydro-2H-pyran-2-yl)-3-(3-chlorobenzamido)propane-1,2-diyl diacetate (269_int-2) (750 mg, 0.95 mmol) in anhydrous dichloromethane (7 mL) was added trifluoro acetic acid (3 mL) dropwise at 0° C. and the resulting reaction mixture was stirred at room temperature for 3 h. After completion, the solvent was concentrated to get crude which was purified by prep-HPLC using acetonitrile in water (+0.05% TFA) as eluents to afford the desired product (1R,2R)-1-((2R,3R,4S,6R)-3-acetamido-4-acetoxy-6-(methoxycarbonyl)-6-((6-((2,2,2-trifluoroacetyl)-14-azaneyl)hexyl)oxy)tetrahydro-2H-pyran-2-yl)-3-(3-chlorobenzamido)propane-1,2-diyl diacetate (269_int-3) as a white solid, Yield: 590 mg, 90%; LCMS (ESI+) m/z 686.6 [M+H]⁺.

### Synthesis of (1R,2R)-1-((2R,3R,4S,6R)-3-acetamido-4-acetoxy-6-((2,2-dimethyl-4,21-dioxo-3,8,11,14,17,20-hexaoxa-5,22-diazaoctacosan-28-yl) oxy)-6-(methoxycarbonyl) tetrahydro-2H-pyran-2-yl)-3-(3-chlorobenzamido)propane-1,2-diyl diacetate (4)

To a stirred solution of (1R,2R)-1-((2R,3R,4S,6R)-3-acetamido-4-acetoxy-6-(methoxycarbonyl)-6-((6-((2,2,2-trifluoroacetyl)-14-azaneyl)hexyl)oxy)tetrahydro-2H-pyran-2-yl)-3-(3-chlorobenzamido)propane-1,2-diyl diacetate (269_Int-3) (590 mg g, 0.85 mmol) in anhydrous dichloromethane (5 mL) was added DIPEA (0.7 mL g, 4.25 mmol) and reaction mixture was cooled to 0° C. and tert-butyl (2-(2-(((4-nitrophenoxy)carbonyl)oxy)ethoxy)ethyl)carbamate (3) (377 mg, 1.02 mmol) was added at 0° C. reaction mixture and was stirred at the room temperature for 1 h. After completion, the reaction mixture was concentrated under reduced pressure to get crude material as afford desired product (1R,2R)-1-((2R,3R,4S,6R)-3-acetamido-4-acetoxy-6-((2,2-dimethyl-4,21-dioxo-3,8,11,14,17,20-hexaoxa-5,22-diazaoctacosan-28-yl) oxy)-6-(methoxycarbonyl) tetrahydro-2H-pyran-2-yl)-3-(3-chlorobenzamido) propane-1,2-diyl diacetate (4) as yellow solid, Yield: 400 mg.

### Synthesis of (2R,4S,5R,6R)-5-acetamido-6-((1R,2R)-3-(3-chlorobenzamido)-1,2-dihydroxypropyl)-2-((2,2-dimethyl-4,21-dioxo-3,8,11,14,17,20-hexaoxa-5,22-diazaoctacosan-28-yl) oxy)-4-hydroxytetrahydro-2H-pyran-2-carboxylic acid (5)

To a stirred solution of (1R,2R)-1-((2R,3R,4S,6R)-3-acetamido-4-acetoxy-6-((2,2-dimethyl-4,21-dioxo-3,8,11,14,17,20-hexaoxa-5,22-diazaoctacosan-28-yl)oxy)-6-(methoxycarbonyl)tetrahydro-2H-pyran-2-yl)-3-(3-chlorobenzamido)propane-1,2-diyl diacetate (5) (400 mg, 0.38 mmol) in methanol (2 mL) and water (1 mL) was added NaOMe (25% in MeOH) (0.41 mL, 1.90 mmol) dropwise at 0° C. The resulting reaction mixture was stirred at room temperature for 16 h. After completion, the reaction mixture was treated with acidic (Dowex 50, H⁺) to maintain pH˜6 and the suspension was filtered. The filtrate was concentrated under reduced pressure and the crude was purified by C18 reverse phase using water: acetonitrile (+0.05% TFA) as eluents to afford the desired ((2R,4S,5R,6R)-5-acetamido-6-((1R,2R)-3-(3-chlorobenzamido)-1,2-dihydroxypropyl)-2-((2,2-dimethyl-4,21-dioxo-3,8,11,14,17,20-hexaoxa-5,22-diazaoctacosan-28-yl)oxy)-4-hydroxytetrahydro-2H-pyran-2-carboxylic acid (5) as off-white solid, Yield: 250 mg, 72%; LCMS (ESI−) m/z 907.8 [M−H]⁻.

### Synthesis of (2R,4S,5R,6R)-5-acetamido-2-((1-amino-16-oxo-3,6,9,12,15-pentaoxa-17-azatricosan-23-yl) oxy)-6-((1R,2R)-3-(3-chlorobenzamido)-1,2-dihydroxypropyl)-4-hydroxytetrahydro-2H-pyran-2-carboxylic acid (6)

To a stirred solution of ((2R,4S,5R,6R)-5-acetamido-6-((1R,2R)-3-(3-chlorobenzamido)-1,2-dihydroxypropyl)-2-((2,2-dimethyl-4,21-dioxo-3,8,11,14,17,20-hexaoxa-5,22-diazaoctacosan-28-yl)oxy)-4-hydroxytetrahydro-2H-pyran-2-carboxylic acid (5) (250 mg, 0.27 mmol) in anhydrous dichloromethane (2.5 mL) was added trifluoro acetic acid (1.5 mL) dropwise at 0° C. and the resulting reaction mixture was stirred at room temperature for 3 h. After completion, the solvent was concentrated to get crude which was purified by prep-HPLC using acetonitrile in water (+0.05% TFA) as eluents to afford the desired product (2R,4S,5R,6R)-5-acetamido-2-((1-amino-16-oxo-3,6,9,12,15-pentaoxa-17-azatricosan-23-yl)oxy)-6-((1R,2R)-3-(3-chlorobenzamido)-1,2-dihydroxypropyl)-4-hydroxytetrahydro-2H-pyran-2-carboxylic acid (6) as a white solid, Yield: 200 mg, 90%. LCMS (ESI+) m/z 809.6 [M+H]⁺.

### Synthesis of (2R,4S,5R,6R)-5-acetamido-6-((1R,2R)-3-(3-chlorobenzamido)-1,2-dihydroxypropyl)-2-((2,19-dioxo-6,9,12,15,18-pentaoxa-3,20-diazahexacosan-26-yl) oxy)-4-hydroxytetrahydro-2H-pyran-2-carboxylic acid (268)

To a stirred solution of (2R,4S,5R,6R)-5-acetamido-2-((1-amino-16-oxo-3,6,9,12,15-pentaoxa-17-azatricosan-23-yl)oxy)-6-((1R,2R)-3-(3-chlorobenzamido)-1,2-dihydroxypropyl)-4-hydroxytetrahydro-2H-pyran-2-carboxylic acid (6) (200 mg, 0.24 mmol) in DMF (2 mL) was added 2,5-dioxopyrrolidin-1-yl acetate (7) (38 mg, 0.24 mmol) and triethylamine (0.18 mL, 1.2 mmol) at 0° C. The resulting reaction mixture was stirred at the same temperature for 1 h. After completion, the reaction mixture was purified by prep-HPLC purification using 15-80% acetonitrile in water with (+0.05% TFA) to afford the desired product (2R,4S,5R,6R)-5-acetamido-6-((1R,2R)-3-(3-chlorobenzamido)-1,2-dihydroxypropyl)-2-((2,19-dioxo-6,9,12,15,18-pentaoxa-3,20-diazahexacosan-26-yl)oxy)-4-hydroxytetrahydro-2H-pyran-2-carboxylic acid (268) as off white solid, Yield: 68 mg, 33%. LCMS (ESI+) m/z 851 [M+H]⁺. ¹H NMR (400 MHz, MeOD): δ 7.88 (t, 1H), 7.77 (dt, J=7.6, 1.2 Hz, 1H), 7.56 (dt, J=8.0, 1.2 Hz, 1H), δ 7.47 (t, 1H), 4.16 (t, J=12.7 Hz, 2H), 4.06-4.01 (m, 1H), 3.99-3.89 (m, 1H), 3.83-3.77 (m, 3H), 3.74-3.60 (m, 15H), 3.55 (t, J=10.8 Hz, 3H), 3.48-3.42 (m, 2H), 3.40-3.28 (m, 3H), 3.1 (t, J=14.0 Hz, 2H), 2.73 (dd, J=12.7, 4.1 Hz, 1H), 2.01 (s, 3H), 1.96 (s, 3H), 1.75 (t, J=24.4 Hz, 1H), 1.61-1.45 (m, 4H), 1.42-1.32 (m, 4H).

### (2R,4S,5R,6R)-5-acetamido-6-((1R,2R)-3-(3-chlorobenzamido)-1,2-dihydroxypropyl)-2-((2,19-dioxo-6,9,12,15-tetraoxa-3,18,20-triazahexacosan-26-yl) oxy)-4-hydroxytetrahydro-2H-pyran-2-carboxylic acid (Cpd. 269)

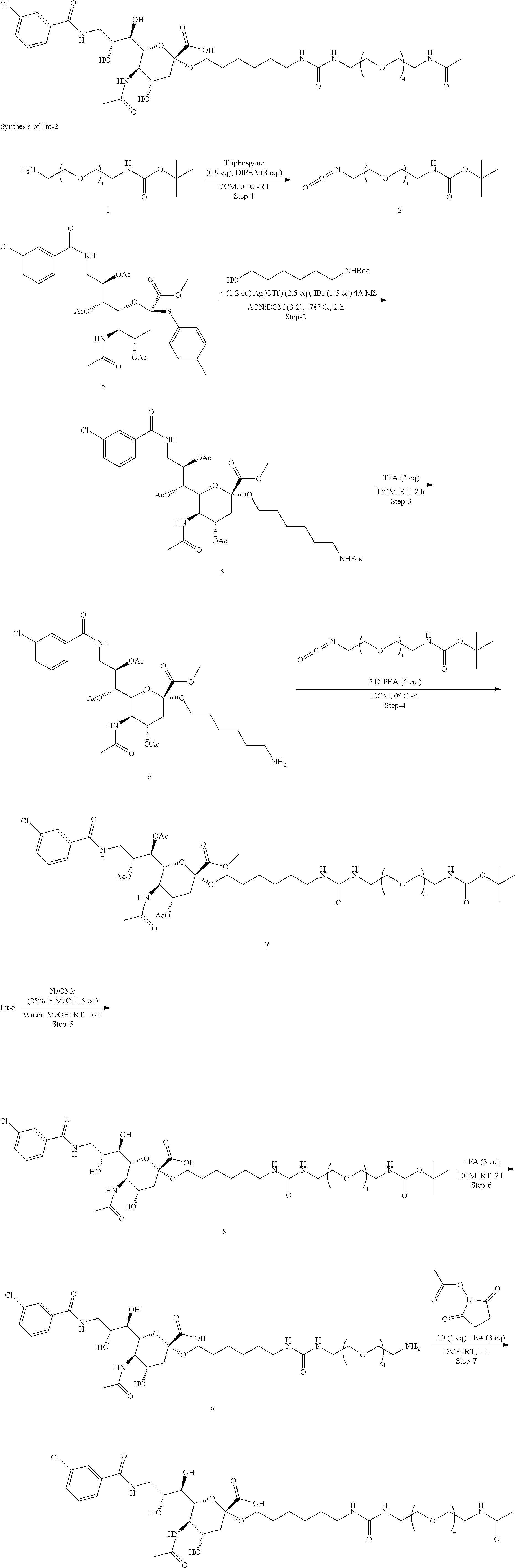

### Synthesis of tert-butyl (14-isocyanato-3,6,9,12-tetraoxatetradecyl) carbamate (2)

To a stirred solution of tert-butyl (14-amino-3,6,9,12-tetraoxatetradecyl) carbamate (1) (500 mg, 1.49 mmol) in anhydrous dichloromethane (5 ml) was added N, N-Diisopropylethylamine (DIPEA) (0.8 mL, 4.46 mmol) followed by addition of Triphosgene (396 mg, 1.34 mmol) at 0° C. and the resulting reaction mixture was stirred at room temperature for next 1 h. After completion, the reaction mixture was concentrated under reduced pressure to get the crude material (600 mg) was directly used for the next step.

### Synthesis of (1R,2R)-1-((2R,3R,4S,6R)-3-acetamido-4-acetoxy-6-((6-((tert-butoxycarbonyl) amino)hexyl)oxy)-6-(methoxycarbonyl) tetrahydro-2H-pyran-2-yl)-3-(3-chlorobenzamido) propane-1,2-diyl diacetate (5)

To a stirred solution of (1R,2R)-1-((2R,3R,4S,6R)-3-acetamido-4-acetoxy-6-(methoxycarbonyl)-6-(p-tolylthio)tetrahydro-2H-pyran-2-yl)-3-(3-chlorobenzamido)propane-1,2-diyl diacetate (3) (3 g, 4.33 mmol), tert-butyl (6-hydroxyhexyl)carbamate (4) (1.13 g, 5.19 mmol) and Silver (1+) trifluoro methane sulfonate (AgOTf) (2.78 g, 10.82 mmol) in anhydrous dichloromethane (12 mL) and anhydrous acetonitrile (18 mL) was added activated 4 Å powdered molecular sieves (4 g) and the reaction mixture was stirred at room temperature for 1 h under nitrogen atmosphere. Then the solution was cooled to −78° C., iodine monobromide (1.34 g, 6.49 mmol) dissolved in dichloromethane (4 mL) was added dropwise and the resulting mixture was stirred at same temperature for 2 h. After completion, the reaction mixture was quenched by addition of triethylamine (5 mL) and allowed to stir at room temperature for 1 h. Then the reaction mixture was filtered, filtrate was washed with aqueous sodium thiosulfate solution, dried over sodium sulfate, filtered, and concentrated under reduced pressure. The crude residue was purified by C18 reverse phase column chromatography using water: acetonitrile (+0.05% TFA) as eluents to afford the desired (1R,2R)-1-((2R,3R,4S,6R)-3-acetamido-4-acetoxy-6-((6-((tert-butoxycarbonyl)amino)hexyl)oxy)-6-(methoxycarbonyl) tetrahydro-2H-pyran-2-yl)-3-(3-chlorobenzamido) propane-1,2-diyl diacetate (5) as off-white solid, Yield: 1.5 g, 44%, LCMS (ESI±) m/z 686.6 [M−100]⁺.

### Synthesis of (1R,2R)-1-((2R,3R,4S,6R)-3-acetamido-4-acetoxy-6-((6-aminohexyl)oxy)-6-(methoxycarbonyl) tetrahydro-2H-pyran-2-yl)-3-(3-chlorobenzamido)propane-1,2-diyl diacetate (6)

To a stirred solution of (1R,2R)-1-((2R,3R,4S,6R)-3-acetamido-4-acetoxy-6-((6-((tert-butoxycarbonyl)amino)hexyl)oxy)-6-(methoxycarbonyl) tetrahydro-2H-pyran-2-yl)-3-(3-chlorobenzamido) propane-1,2-diyl diacetate (5) (1.5 g, 1.91 mmol) in anhydrous dichloromethane (15 mL) was added trifluoro acetic acid (0.5 mL) at 0° C. The reaction mixture was stirred at room temperature for 2 h. After completion, the reaction mixture was concentrated to get the crude which was dissolved in methanol and neutralized with Tetra alkyl ammonium carbonate, the polymer-bound resin. Then the reaction mixture was filtered and filtrate was concentrated to afford (1R,2R)-1-((2R,3R,4S,6R)-3-acetamido-4-acetoxy-6-((6-aminohexyl)oxy)-6-(methoxycarbonyl) tetrahydro-2H-pyran-2-yl)-3-(3-chlorobenzamido) propane-1,2-diyl diacetate (6) as off-white solid, Yield-700 mg, 53%; LCMS (ESI±) m/z 686.6 [M+H]⁺.

### Synthesis of (1R,2R)-1-((2R,3R,4S,6R)-3-acetamido-4-acetoxy-6-((2,2-dimethyl-4,21-dioxo-3,8,11,14,17-pentaoxa-5,20,22-triazaoctacosan-28-yl) oxy)-6-(methoxycarbonyl) tetrahydro-2H-pyran-2-yl)-3-(3-chlorobenzamido) propane-1,2-diyl diacetate (7)

To a stirred solution of (1R,2R)-1-((2R,3R,4S,6R)-3-acetamido-4-acetoxy-6-((6-aminohexyl)oxy)-6-(methoxycarbonyl)tetrahydro-2H-pyran-2-yl)-3-(3-chlorobenzamido) propane-1,2-diyl diacetate (6) (700 mg, 1.02 mmol) in anhydrous dichloromethane (7 mL) was added N, N-Diisopropylethylamine (DIPEA) (0.9 mL, 5.1 mmol) followed by addition of tert-butyl (14-isocyanato-3,6,9,12-tetraoxatetradecyl)carbamate (2) (407 mg, 1.12 mmol) at 0° C. and the resulting reaction mixture was stirred at room temperature for next 2 h. After completion, the reaction mixture was concentrated to get the crude residue which was further purified by C18 reverse phase column chromatography using water: acetonitrile (+0.05% TFA) as eluents to afford the desired (1R,2R)-1-((2R,3R,4S,6R)-3-acetamido-4-acetoxy-6-((2,2-dimethyl-4,21-dioxo-3,8,11,14,17-pentaoxa-5,20,22-triazaoctacosan-28-yl) oxy)-6-(methoxycarbonyl) tetrahydro-2H-pyran-2-yl)-3-(3-chlorobenzamido) propane-1,2-diyl diacetate (7) as off-White solid, Yield-400 mg, 40%, LCMS (ESI±) m/z 1047.0 [M−H]⁻.

### Synthesis of (2R,4S,5R,6R)-5-acetamido-6-((1R,2R)-3-(3-chlorobenzamido)-1,2-dihydroxypropyl)-2-((2,2-dimethyl-4,21-dioxo-3,8,11,14,17-pentaoxa-5,20,22-triaza octacosan-28-yl) oxy)-4-hydroxytetrahydro-2H-pyran-2-carboxylic acid (8)

To a stirred solution of (1R,2R)-1-((2R,3R,4S,6R)-3-acetamido-4-acetoxy-6-((2,2-dimethyl-4,21-dioxo-3,8,11,14,17-pentaoxa-5,20,22-triazaoctacosan-28-yl) oxy)-6-(methoxycarbonyl) tetrahydro-2H-pyran-2-yl)-3-(3-chlorobenzamido)propane-1,2-diyl diacetate (7) (400 mg, 0.38 mmol) in methanol (4 mL) and water (1 mL) was added 25% sodium methoxide in methanol (0.07 mL, 1.91 mmol) at 0° and the reaction mixture was stirred at room temperature for 16 h. After completion the reaction mixture was treated with acidic (Dowex 50, H⁺) to make the pH˜6 and the suspension was filtered, filtrate was concentrated to get (2R,4S,5R,6R)-5-acetamido-6-((1R,2R)-3-(3-chlorobenzamido)-1,2-dihydroxypropyl)-2-((2,2-dimethyl-4,21-dioxo-3,8,11,14,17-pentaoxa-5,20,22-triazaoctacosan-28-yl) oxy)-4-hydroxytetrahydro-2H-pyran-2-carboxylic acid (8) as off white solid, Yield: 290 mg, 83%, LCMS (ESI±) m/z 906.7 [M−H]⁻.

### Synthesis of (2R,4S,5R,6R)-5-acetamido-2-((1-amino-16-oxo-3,6,9,12-tetraoxa-15,17-diazatricosan-23-yl) oxy)-6-((1R,2R)-3-(3-chlorobenzamido)-1,2-dihydroxypropyl)-4-hydroxytetrahydro-2H-pyran-2-carboxylic acid (9)

To a stirred solution of (2R,4S,5R,6R)-5-acetamido-6-((1R,2R)-3-(3-chlorobenzamido)-1,2-dihydroxypropyl)-2-((2,2-dimethyl-4,21-dioxo-3,8,11,14,17-pentaoxa-5,20,22-triazaoctacosan-28-yl) oxy)-4-hydroxytetrahydro-2H-pyran-2-carboxylic acid (8) (290 mg, 0.319 mmol) in anhydrous dichloromethane (3 mL) was added trifluoro acetic acid (0.07 mL) at 0° C. The reaction mixture was stirred at room temperature for 2 h. After completion, the reaction mixture was concentrated to get the crude which was dissolved in methanol and neutralized with Tetra alkyl ammonium carbonate, the polymer-bound resin. Then the reaction mixture was filtered and filtrate was concentrated to afford (2R,4S,5R,6R)-5-acetamido-2-((1-amino-16-oxo-3,6,9,12-tetraoxa-15,17-diazatricosan-23-yl)oxy)-6-((1R,2R)-3-(3-chloro benzamido)-1,2-dihydroxypropyl)-4-hydroxytetrahydro-2H-pyran-2-carboxylic acid (9) as off-white solid, Yield-200 mg, 77%. LCMS (ESI±) m/z 808.6 [M+H]⁺.

### Synthesis of (2R,4S,5R,6R)-5-acetamido-6-((1R,2R)-3-(3-chlorobenzamido)-1,2-dihydroxypropyl)-2-((2,19-dioxo-6,9,12,15-tetraoxa-3,18,20-triazahexacosan-26-yl) oxy)-4-hydroxytetrahydro-2H-pyran-2-carboxylic acid (269)

To a stirred solution of (2R,4S,5R,6R)-5-acetamido-2-((1-amino-16-oxo-3,6,9,12-tetraoxa-15,17-diazatricosan-23-yl)oxy)-6-((1R,2R)-3-(3-chlorobenzamido)-1,2-dihydroxypropyl)-4-hydroxytetrahydro-2H-pyran-2-carboxylic acid (9) (200 mg, 0.25 mmol) in DMF (2 mL) were added triethylamine (0.1 mL, 0.742 mmol) followed by 2,5-dioxopyrrolidin-1-yl acetate (10) (39 mg, 0.25 mmol) at 0° C. The resulting reaction mixture was stirred at room temperature for 1 h. After completion, the reaction mixture was concentrated and purified by prep-HPLC purification by using acetonitrile and water (+0.05%, TFA) to afford the desired product (2R,4S,5R,6R)-5-acetamido-6-((1R,2R)-3-(3-chlorobenzamido)-1,2-dihydroxypropyl)-2-((2,19-dioxo-6,9,12,15-tetraoxa-3,18,20-triazahexacosan-26-yl)oxy)-4-hydroxytetrahydro-2H-pyran-2-carboxylic acid (269) as white solid, Yield: 72 mg, 34%. LCMS (ESI+) m/z 850.5 [M+H]⁺. ¹H NMR (400 MHz, MeOD): δ 7.87 (s, 1H), 7.77 (d, J=7.6, 1H), 7.55 (d, J=8.4, 1H), 7.46 (t, J=15.6, 1H), 4.02-3.91 (m, 1H), 3.82-3.76 (m, 5H), 3.74-3.70 (m, 14H), 3.57-3.51 (m, 6H), 3.48-3.40 (m, 3H), 3.10 (t, J=14, 2H), 2.71 (dd, J=12.4, 4.4 Hz, 1H), 2.00 (s, 3H), 1.96 (s, 3H), 1.74-1.67 (t, J=24.4, 1H), 1.58-1.55 (m, 2H), 1.49-1.43 (m, 2H),

1.36-1.32 (m, 4H).

### (2R,4S,5R,6R)-5-acetamido-6-((1R,2R)-3-(3-chlorobenzamido)-1,2-dihydroxypropyl)-2-((2,19-dioxo-6,9,12,15-tetraoxa-3,18-diazapentacosan-25-yl) oxy)-4-hydroxytetrahydro-2H-pyran-2-carboxylic acid (Cpd. 270)

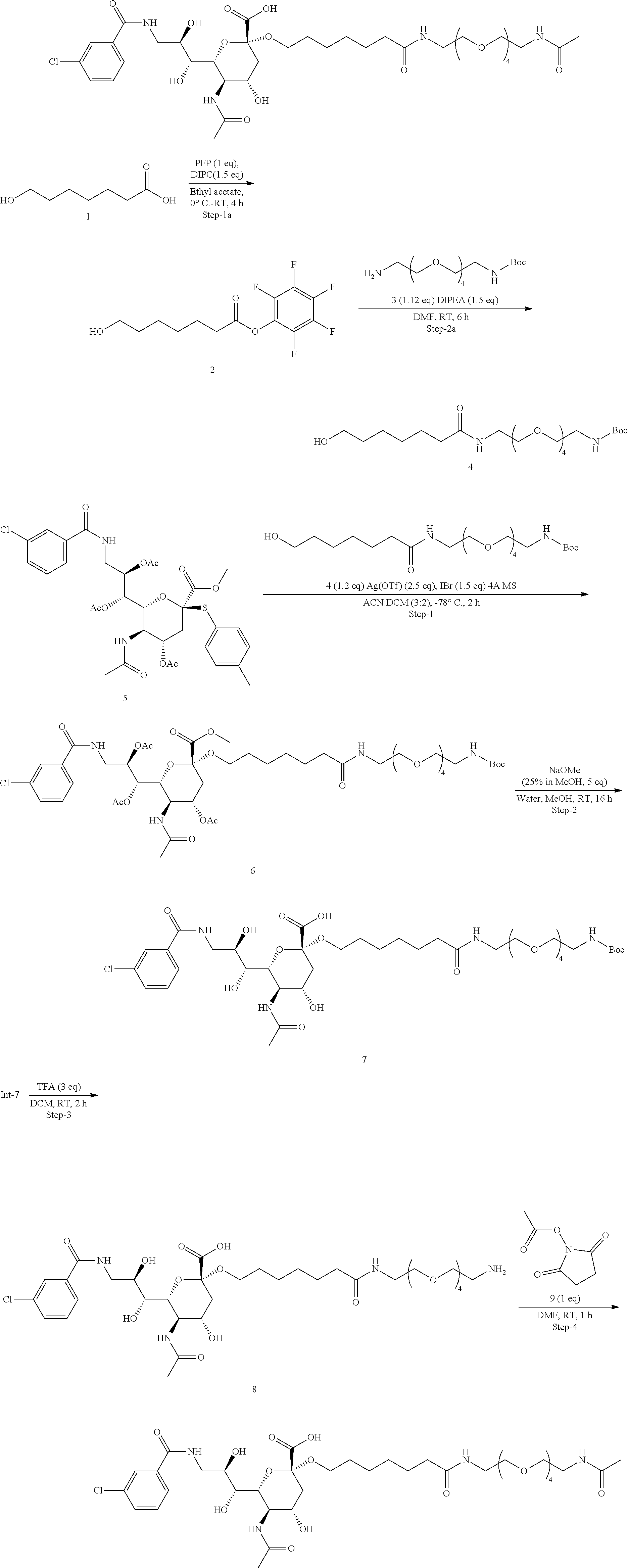

### Synthesis of perfluorophenyl 7-hydroxyheptanoate (2)

To a stirred solution of 7-hydroxyheptanoic acid (1) (5 g, 34 mmol) in EtOAc (50 mL) was added 2,3,4,5,6-pentafluorophenol (6.25 g, 34 mmol), followed by dropwise addition of N, N′-Di-isopropyl carbodiimide (6.43 g, 51 mmol) at 0° C. The resulting reaction mixture was stirred for 4 h at the same temperature. After completion, the reaction mixture was filtered, residue was washed with ethyl acetate, filtrate was concentrated under reduced pressure to afford of the desired product perfluorophenyl 7-hydroxyheptanoate (2), which was used as such for the next step, Yield: 7 g, 65%.

### Synthesis of tert-butyl (22-hydroxy-16-oxo-3,6,9,12-tetraoxa-15-azadocosyl) carbamate (4)

To a stirred solution of perfluorophenyl 7-hydroxyheptanoate (2) (7 g, 22 mmol) in DMF (70 mL) were added tert-butyl (14-amino-3,6,9,12-tetraoxatetradecyl)carbamate (4) (7.5 g, 22 mmol), DIPEA (6 mL, 33 mmol) at 0° C. and the reaction mixture was stirred at the same temperature for next 2 h. After completion, the reaction mixture was purified by C18 reverse phase column chromatography using water: acetonitrile (+0.05% TFA) as eluents to afford the desired product tert-butyl (22-hydroxy-16-oxo-3,6,9,12-tetraoxa-15-azadocosyl) carbamate (4) as off-white solid. Yield: 7 g, 67%; LCMS (ESI+) m/z 465.5 [M+H]⁺.

### Synthesis of (1R,2R)-1-((2R,3R,4S,6R)-3-acetamido-4-acetoxy-6-((2,2-dimethyl-4,21-dioxo-3,8,11,14,17-pentaoxa-5,20-diazaheptacosan-27-yl) oxy)-6-(methoxycarbonyl)tetrahydro-2H-pyran-2-yl)-3-(3-chlorobenzamido)propane-1,2-diyl diacetate (6)

To a stirred solution of (1R,2R)-1-((2R,3R,4S,6R)-3-acetamido-4-acetoxy-6-(methoxycarbonyl)-6-(p-tolylthio)tetrahydro-2H-pyran-2-yl)-3-(3-chlorobenzamido)propane-1,2-diyl diacetate (5) (1.5 g, 2.16 mmol), tert-butyl (22-hydroxy-16-oxo-3,6,9,12-tetraoxa-15-azadocosyl)carbamate (4) (1.21 g, 2.60 mmol) and Silver (1+) trifluoro methane sulfonate (AgOTf) (1.39 g, 5.4 mmol) in anhydrous dichloromethane (12 mL) and anhydrous acetonitrile (18 mL) was added activated 4 Å powdered molecular sieves (5 g) and the reaction mixture was stirred at room temperature for 1 h under nitrogen atmosphere. Then the solution was cooled to −78° C., iodine monobromide (670 mg, 3.24 mmol) dissolved in dichloromethane (4 mL) was added dropwise, and the resulting mixture was stirred at same temperature for 2 h. After completion, the reaction mixture was quenched by addition of triethylamine (5 mL) and allowed to stir at room temperature for 1 h. Then the reaction mixture was filtered, the filtrate was washed with aqueous sodium thiosulfate solution, dried over sodium sulfate, filtered, and concentrated under reduced pressure. The crude residue obtained was purified by C18 reverse phase column chromatography using water: acetonitrile (+0.05% TFA) as eluents to afford the desired (1R,2R)-1-((2R,3R,4S,6R)-3-acetamido-4-acetoxy-6-((2,2-dimethyl-4,21-dioxo-3,8,11,14,17-pentaoxa-5,20-diazaheptacosan-27-yl)oxy)-6-(methoxycarbonyl)tetrahydro-2H-pyran-2-yl)-3-(3-chlorobenzamido)propane-1,2-diyl diacetate (6) as off-white solid, Yield: 680 mg, 30.40%; LCMS (ESI+) m/z 1033.8 [M+H]⁺.

### Synthesis of (2R,4S,5R,6R)-5-acetamido-6-((1R,2R)-3-(3-chlorobenzamido)-1,2-dihydroxypropyl)-2-((2,2-dimethyl-4,21-dioxo-3,8,11,14,17-pentaoxa-5,20-diazaheptacosan-27-yl) oxy)-4-hydroxytetrahydro-2H-pyran-2-carboxylic acid (7)

To a stirred solution of (1R,2R)-1-((2R,3R,4S,6R)-3-acetamido-4-acetoxy-6-((2,2-dimethyl-4,21-dioxo-3,8,11,14,17-pentaoxa-5,20-diazaheptacosan-27-yl)oxy)-6-(methoxycarbonyl)tetrahydro-2H-pyran-2-yl)-3-(3-chlorobenzamido)propane-1,2-diyl diacetate (6) (680 mg, 0.66 mmol) in methanol (7 mL) and water (3 mL) was added 25% sodium methoxide in methanol (0.22 mL, 3.67 mmol) at 0° C. and the reaction mixture was stirred at room temperature for 16 h. After completion the reaction mixture was treated with acidic (Dowex 50, H⁺) to make the pH˜6 and the suspension was filtered, The filtrate was concentrated under reduced pressure and the crude was purified by C18 reverse phase using water: acetonitrile (+0.05% TFA) as eluents to afford the desired (2R,4S,5R,6R)-5-acetamido-6-((1R,2R)-3-(3-chlorobenzamido)-1,2-dihydroxypropyl)-2-((2,2-dimethyl-4,21-dioxo-3,8,11,14,17-pentaoxa-5,20-diazaheptacosan-27-yl)oxy)-4-hydroxytetrahydro-2H-pyran-2-carboxylic acid (7) as off-white solid, Yield: 410 mg, 69.75%; LCMS (ESI±) m/z 891.8 [M−H]⁻.

### Synthesis of (2R,4S,5R,6R)-5-acetamido-2-((1-amino-16-oxo-3,6,9,12-tetraoxa-15-azadocosan-22-yl) oxy)-6-((1R,2R)-3-(3-chlorobenzamido)-1,2-dihydroxypropyl)-4-hydroxytetrahydro-2H-pyran-2-carboxylic acid (8)

To a stirred solution of (2R,4S,5R,6R)-5-acetamido-6-((1R,2R)-3-(3-chlorobenzamido)-1,2-dihydroxypropyl)-2-((2,2-dimethyl-4,21-dioxo-3,8,11,14,17-pentaoxa-5,20-diazaheptacosan-27-yl)oxy)-4-hydroxytetrahydro-2H-pyran-2-carboxylic acid (7) (410 mg, 0.46 mmol) in anhydrous dichloromethane (4 mL) was added trifluoro acetic acid (1 mL) at 0° C. The reaction mixture was stirred at room temperature for 2 h. After completion, the reaction mixture was concentrated to get the crude which was dissolved in methanol and neutralized with Tetra alkyl ammonium carbonate, the polymer-bound resin. Then the reaction mixture was filtered and filtrate was concentrated to afford (2R,4S,5R,6R)-5-acetamido-2-((1-amino-16-oxo-3,6,9,12-tetraoxa-15-azadocosan-22-yl)oxy)-6-((1R,2R)-3-(3-chlorobenzamido)-1,2-dihydroxypropyl)-4-hydroxytetrahydro-2H-pyran-2-carboxylic acid (8) as off-white solid, Yield-230 mg, 63.18%; LCMS (ESI+) m/z 793.6 [M+H]⁺.

### Synthesis of (2R,4S,5R,6R)-5-acetamido-6-((1R,2R)-3-(3-chlorobenzamido)-1,2-dihydroxypropyl)-2-((2,19-dioxo-6,9,12,15-tetraoxa-3,18-diazapentacosan-25-yl) oxy)-4-hydroxytetrahydro-2H-pyran-2-carboxylic acid (270)

To a stirred solution of (2R,4S,5R,6R)-5-acetamido-2-((1-amino-16-oxo-3,6,9,12-tetraoxa-15-azadocosan-22-yl)oxy)-6-((1R,2R)-3-(3-chlorobenzamido)-1,2-dihydroxypropyl)-4-hydroxytetrahydro-2H-pyran-2-carboxylic acid (8) (100 mg, 0.13 mmol) in DMF (1 mL) were added 2,5-dioxopyrrolidin-1-yl acetate (8) (20 mg, 0.13 mmol) at 0° C. The resulting reaction mixture was stirred at room temperature for 1 h. After completion, the reaction mixture was concentrated and purified by prep-HPLC purification by using acetonitrile and water (+0.05%, TFA) to afford the desired product (2R,4S,5R,6R)-5-acetamido-6-((1R,2R)-3-(3-chlorobenzamido)-1,2-dihydroxypropyl)-2-((2,19-dioxo-6,9,12,15-tetraoxa-3,18-diazapentacosan-25-yl)oxy)-4-hydroxytetrahydro-2H-pyran-2-carboxylic acid (270) as a white solid, Yield: 34 mg, 32.29%; LCMS (ESI±) m/z 835.5 [M+H]⁺. ¹H NMR (400 MHz, MeOD): δ 7.88 (t, 1H), 7.77 (dt, J=7.6, 1.2 Hz, 1H), 7.56 (dt, J=8.0, 1.2 Hz, 1H), δ 7.47 (t, 1H), 4.07-4.01 (m, 1H), 3.83-3.69 (m, 4H), 3.66-3.53 (m, 12H), 3.58-3.53 (m, 5H), 3.48-3.32 (m, 7H), 2.73 (dd, J=12.7, 4.1 Hz, 1H), 2.20 (t, J=14.8 Hz, 2H), 2.01 (s, 3H), 1.96 (s, 3H), 1.74 (t, 1H), 1.57-1.48 (m, 4H), 1.45-1.32 (m, 4H).

### (2R,4S,5R,6R)-5-acetamido-6-((1R,2R)-3-(3-chlorobenzamido)-1,2-dihydroxypropyl)-2-((2,18-dioxo-6,9,12,15-tetraoxa-3,19-diazapentacosan-25-yl) oxy)-4-hydroxytetrahydro-2H-pyran-2-carboxylic acid (Cpd. 271)

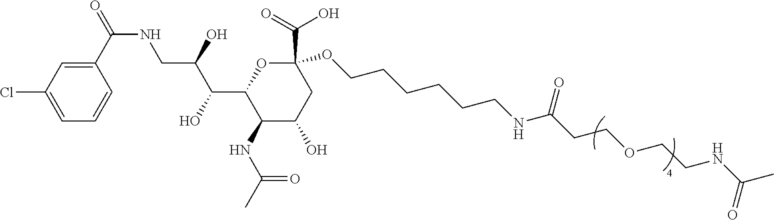

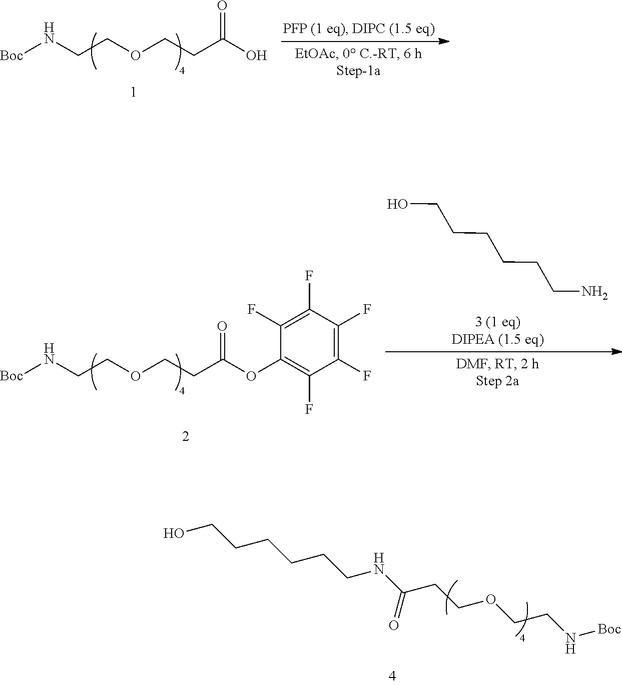

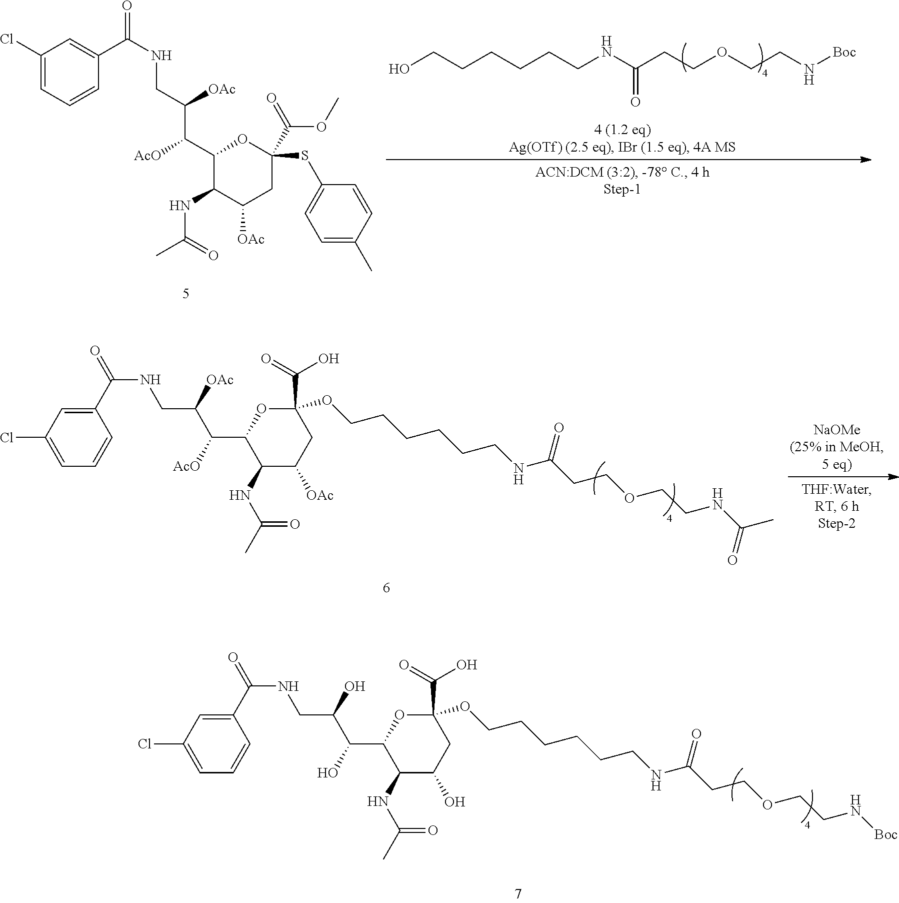

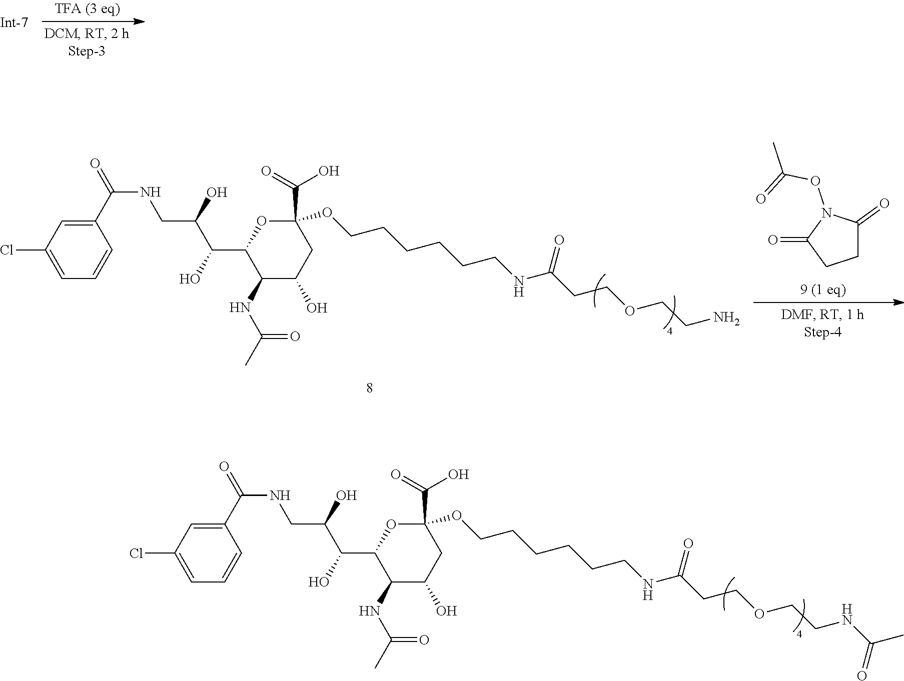

### Synthesis of perfluorophenyl 2,2-dimethyl-4-oxo-3,8,11,14,17-pentaoxa-5-azaicosan-20-oate (2)

To a stirred solution of 2,2-dimethyl-4-oxo-3,8,11,14,17-pentaoxa-5-azaicosan-20-oic acid (1) (5 g, 13.6 mmol) and penta-fluoro phenol (2.5 g, 13.6 mmol) in Ethyl acetate (50 mL) was added di-iso-propyl carbodimide (2.58 g, 20.4 mmol) dropwise at 0° C. Then the reaction mixture was stirred at room temperature for 6 h. After completion, the reaction mixture was filtered, filtrate was concentrated, the crude residue was purified by Silica gel column chromatography by using Ethyl acetate and Hexane as eluents to afford perfluorophenyl 2,2-dimethyl-4-oxo-3,8,11,14,17-pentaoxa-5-azaicosan-20-oate (2) as off-white solid, Yield: 5 g, 69.44%; LCMS (ESI±) m/z 533.65 [M+H]⁺.

### Synthesis of tert-butyl (22-hydroxy-15-oxo-3,6,9,12-tetraoxa-16-azadocosyl) carbamate (4)

To a stirred solution of perfluorophenyl 2,2-dimethyl-4-oxo-3,8,11,14,17-pentaoxa-5-azaicosan-20-oate (2) (4 g, 7.52 mmol) and 6-aminohexan-1-ol (3) (880 mg, 7.52 mmol) in N,N-dimethylformamide 10 mL) was added di-isopropyl ethylamine (2.08 ml, 11.29 mmol) at 0° C. The resulting reaction mixture was stirred at room temperature for 2 h. After completion, the reaction mixture was concentrated and purified by combi-flash column chromatography by using Ethyl acetate and Hexane as eluents to afford tert-butyl (22-hydroxy-15-oxo-3,6,9,12-tetraoxa-16-azadocosyl) carbamate (4) as white solid, Yield: 2.4 g, 70.58%; LCMS (ESI±) m/z 465.5 [M+H]⁺.

### Synthesis of (1R,2R)-1-((2R,3R,4S,6R)-3-acetamido-4-acetoxy-6-((2,2-dimethyl-4,20-dioxo-3,8,11,14,17-pentaoxa-5,21-diazaheptacosan-27-yl) oxy)-6-(methoxycarbonyl)tetrahydro-2H-pyran-2-yl)-3-(3-chlorobenzamido)propane-1,2-diyl diacetate (6)

To a stirred solution of (1R,2R)-1-((2R,3R,4S,6R)-3-acetamido-4-acetoxy-6-(methoxycarbonyl)-6-(p-tolylthio)tetrahydro-2H-pyran-2-yl)-3-(3-chlorobenzamido)propane-1,2-diyl diacetate (5) (1.5 g, 2.16 mmol), tert-butyl (22-hydroxy-15-oxo-3,6,9,12-tetraoxa-16-azadocosyl) carbamate (4) (1.21 g, 2.60 mmol) and Silver (1+) trifluoro methane sulfonate (AgOTf) (1.39 g, 5.4 mmol) in anhydrous dichloromethane (12 mL) and anhydrous acetonitrile (18 mL) was added activated 4 Å powdered molecular sieves (5 g) and the reaction mixture was stirred at room temperature for 1 h under nitrogen atmosphere. Then the solution was cooled to −78° C., iodine monobromide (0.67 g, 3.24 mmol) dissolved in dichloromethane (4 mL) was added dropwise, and the resulting mixture was stirred at same temperature for 2 h. After completion, the reaction mixture was quenched by addition of triethylamine (5 mL) and allowed to stir at room temperature for 1 h. Then the reaction mixture was filtered, the filtrate was washed with aqueous sodium thiosulfate solution, dried over sodium sulfate, filtered, and concentrated under reduced pressure. The crude residue obtained was purified by C18 reverse phase column chromatography using water: acetonitrile (+0.05% TFA) as eluents to afford the desired (1R,2R)-1-((2R,3R,4S,6R)-3-acetamido-4-acetoxy-6-((2,2-dimethyl-4,20-dioxo-3,8,11,14,17-pentaoxa-5,21-diazaheptacosan-27-yl)oxy)-6-(methoxycarbonyl)tetrahydro-2H-pyran-2-yl)-3-(3-chlorobenzamido)propane-1,2-diyl diacetate (6) as off-white solid, Yield: 760 mg, 33.79%; LCMS (ESI±) m/z 1033.4 [M+H]⁺.

### Synthesis of (2R,4S,5R,6R)-5-acetamido-6-((1R,2R)-3-(3-chlorobenzamido)-1,2-dihydroxypropyl)-2-((2,2-dimethyl-4,20-dioxo-3,8,11,14,17-pentaoxa-5,21-diazaheptacosan-27-yl) oxy)-4-hydroxytetrahydro-2H-pyran-2-carboxylic acid (7)

To a stirred solution of (1R,2R)-1-((2R,3R,4S,6R)-3-acetamido-4-acetoxy-6-((2,2-dimethyl-4,20-dioxo-3,8,11,14,17-pentaoxa-5,21-diazaheptacosan-27-yl)oxy)-6-(methoxycarbonyl)tetrahydro-2H-pyran-2-yl)-3-(3-chlorobenzamido)propane-1,2-diyl diacetate (6) (760 mg, 0.73 mmol) in methanol (7 mL) and water (3 mL) was added 25% sodium methoxide in methanol (0.22 mL, 3.67 mmol) at 0° C. and the reaction mixture was stirred at room temperature for 16 h. After completion the reaction mixture was treated with acidic (Dowex 50, H⁺) to make the pH˜6 and the suspension was filtered, The filtrate was concentrated under reduced pressure and the crude was purified by C18 reverse phase using water: acetonitrile (+0.05% TFA) as eluents to afford the desired (2R,4S,5R,6R)-5-acetamido-6-((1R,2R)-3-(3-chlorobenzamido)-1,2-dihydroxypropyl)-2-((2,2-dimethyl-4,20-dioxo-3,8,11,14,17-pentaoxa-5,21-diazaheptacosan-27-yl)oxy)-4-hydroxytetrahydro-2H-pyran-2-carboxylic acid (7) as off-white solid, Yield: 440 mg, 66.98%; LCMS (ESI±) m/z 893.7 [M+H]⁺.

### Synthesis of (2R,4S,5R,6R)-5-acetamido-2-((1-amino-15-oxo-3,6,9,12-tetraoxa-16-azadocosan-22-yl) oxy)-6-((1R,2R)-3-(3-chlorobenzamido)-1,2-dihydroxypropyl)-4-hydroxytetrahydro-2H-pyran-2-carboxylic acid (8)

To a stirred solution of (2R,4S,5R,6R)-5-acetamido-6-((1R,2R)-3-(3-chlorobenzamido)-1,2-dihydroxypropyl)-2-((2,2-dimethyl-4,20-dioxo-3,8,11,14,17-pentaoxa-5,21-diazaheptacosan-27-yl)oxy)-4-hydroxytetrahydro-2H-pyran-2-carboxylic acid (7) (440 mg, 0.49 mmol) in anhydrous dichloromethane (4.4 mL) was added trifluoro acetic acid (1 mL) at 0° C. The reaction mixture was stirred at room temperature for 2 h. After completion, the reaction mixture was concentrated to get the crude which was dissolved in methanol and neutralized with Tetra alkyl ammonium carbonate, the polymer-bound resin. Then the reaction mixture was filtered and filtrate was concentrated to afford (2R,4S,5R,6R)-5-acetamido-2-((1-amino-15-oxo-3,6,9,12-tetraoxa-16-azadocosan-22-yl)oxy)-6-((1R,2R)-3-(3-chlorobenzamido)-1,2-dihydroxypropyl)-4-hydroxytetrahydro-2H-pyran-2-carboxylic acid (8) as off-white solid, Yield-230 mg, 58.87%; LCMS (ESI±) m/z 793.6 [M+H]⁺.

### Synthesis of (2R,4S,5R,6R)-5-acetamido-6-((1R,2R)-3-(3-chlorobenzamido)-1,2-dihydroxypropyl)-2-((2,18-dioxo-6,9,12,15-tetraoxa-3,19-diazapentacosan-25-yl)oxy)-4-hydroxytetrahydro-2H-pyran-2-carboxylic acid (271)

To a stirred solution of (2R,4S,5R,6R)-5-acetamido-2-((1-amino-15-oxo-3,6,9,12-tetraoxa-16-azadocosan-22-yl)oxy)-6-((1R,2R)-3-(3-chlorobenzamido)-1,2-dihydroxypropyl)-4-hydroxytetrahydro-2H-pyran-2-carboxylic acid (8) (100 mg, 0.13 mmol) in DMF (1 mL) were added 2,5-dioxopyrrolidin-1-yl acetate (8) (20 mg, 0.13 mmol) at 0° C. The resulting reaction mixture was stirred at room temperature for 1 h. After completion, the reaction mixture was concentrated and purified by prep-HPLC purification by using acetonitrile and water (+0.05%, TFA) to afford the desired product (2R,4S,5R,6R)-5-acetamido-6-((1R,2R)-3-(3-chlorobenzamido)-1,2-dihydroxypropyl)-2-((2,18-dioxo-6,9,12,15-tetraoxa-3,19-diazapentacosan-25-yl)oxy)-4-hydroxytetrahydro-2H-pyran-2-carboxylic acid (271) as a white solid, Yield: 62 mg, 58.88%; LCMS (ESI±) m/z 835.7 [M+H]⁺. ¹H NMR (400 MHz, MeOD): δ 7.88 (t, 1H), 7.77 (dt, J=7.6, 1.2 Hz, 1H), 7.56 (dt, J=8.0, 1.2 Hz, 1H), δ 7.47 (t, 1H), 4.07-4.01 (m, 2H), 3.83-3.72 (m, 7H), 3.74-3.71 (m, 1H), 3.67-3.58 (m, 10H), 3.56-3.53 (t, J=11.2 Hz, 3H), 3.48-3.42 (m, 2H), 3.17 (t, J=14.0 Hz, 2H), 2.73 (dd, J=12.7, 4.1 Hz, 1H), 2.44 (t, J=14.8 Hz, 2H), 2.01 (s, 3H), 1.96 (s, 3H), 1.74 (t, 1H), 1.57-1.48 (m, 4H), 1.45-1.32 (m, 4H).

### (2R,4S,5R,6R)-5-acetamido-6-((1R,2R)-3-(3-chlorobenzamido)-1,2-dihydroxypropyl)-4-hydroxy-2-((6-(1-(2-oxo-6,9,12,15-tetraoxa-3-azaheptadecan-17-yl)-1H-1,2,3-triazol-4-yl)hexyl)oxy)tetrahydro-2H-pyran-2-carboxylic acid (Cpd. 272)

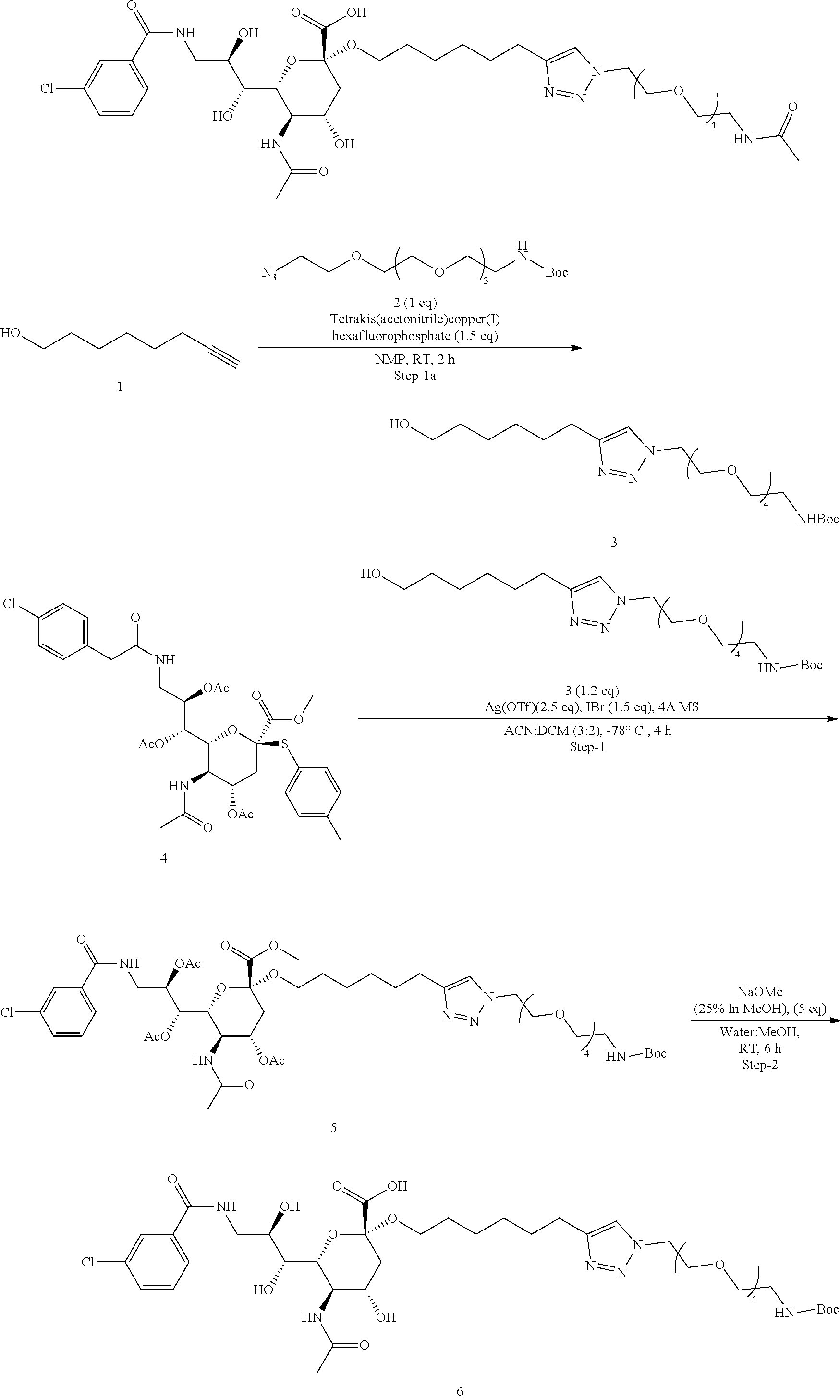

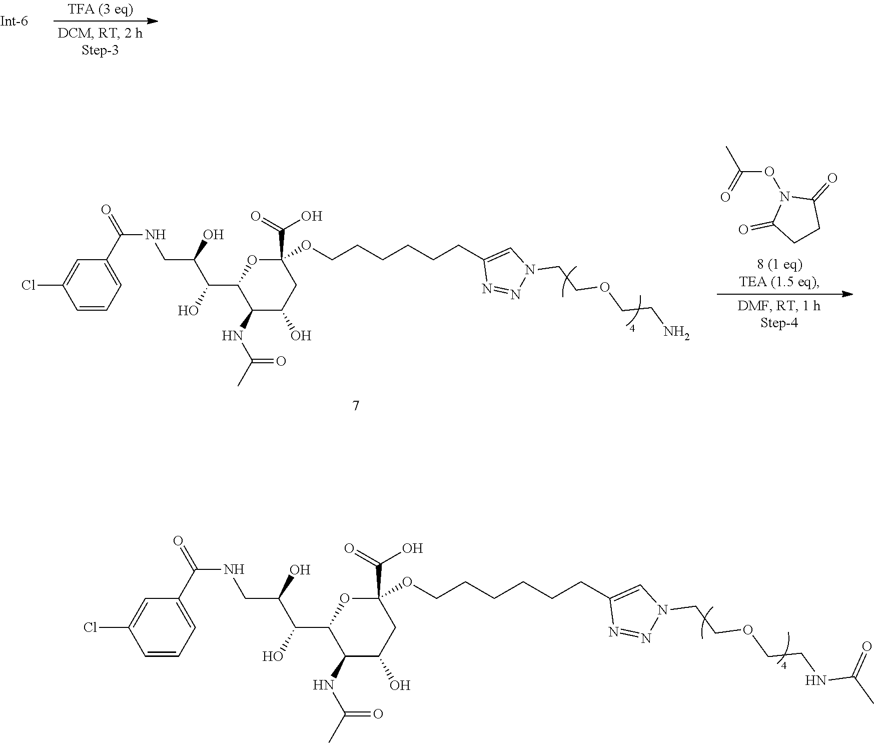

### Synthesis of tert-butyl (14-(4-(6-hydroxyhexyl)-1H-1,2,3-triazol--yl)-3,6,9,12-tetraoxatetradecyl) carbamate (3)

To a stirred solution of oct-7-yn-1-ol (1) (2 g, 15.84 mmol) and tert-butyl (14-azido-3,6,9,12-tetraoxatetradecyl)carbamate (2) (5.74 g, 15.84 mmol) in NMP (20 mL) was added Tetrakis(acetonitrile)copper(I)hexafluorophosphate (7.4 g, 23.7 mmol, 1.5 eq.) and the reaction mixture was stirred at room temperature for next 2 h. After completion, the reaction mixture was concentrated under reduced pressure to get the crude material, which was purified by C18 reverse phase purification using Acetonitrile and water (+0.05% TFA) as eluents to afford the desired compound tert-butyl (14-(4-(6-hydroxyhexyl)-1H-1,2,3-triazol-1-yl)-3,6,9,12-tetraoxatetradecyl)carbamate (3) as colorless solid, Yield: 4.1 g, 52%. ¹H NMR (400 MHz, DMSO) T 7.80 (s, 1H), 6.73 (s, 1H), 4.46 (t, J=5.6 Hz, 2H), 3.79 (t, J=5.6 Hz, 2H), 3.68-3.49 (m, 14H), 3.44-3.35 (m, 6H), 3.082-3.038 (m, 2H), 2.59 (t, J=7.6, Hz, 2H), 1.60-1.53 (m, 2H), 1.42-1.41 (m, 2H), 1.37 (s, 9H), 1.34-1.29 (m, 4H).

### Synthesis of (aR,2R)-1-((2R,3R,4S,6R)-3-acetamido-4-acetoxy-6-((6-(1-(2,2-dimethyl-4-oxo-3,8,11,14,17 pentaoxa-5-azanonadecan-19-yl)-1H-1,2,3-triazol-4-yl)hexyl)oxy)-6-(methoxycarbonyl)tetrahydro-2H-pyran-2-yl)-3-(3-chlorobenzamido)propane-1,2-diyl diacetate (5)

To a stirred solution of (1R,2R)-1-((2R,3R,4S,6R)-3-acetamido-4-acetoxy-6-(methoxycarbonyl)-6-(p-tolylthio)tetrahydro-2H-pyran-2-yl)-3-(3-chlorobenzamido)propane-1,2-diyl diacetate (4) (1.5 g, 2.16 mmol) (14-(4-(6-hydroxyhexyl)-1H-1,2,3-triazol-1-yl)-3,6,9,12-tetraoxatetradecyl) carbamate (3) (1.27 g, 2.60 mmol) and Silver (1+) trifluoro methane sulfonate (AgOTf) (1.39 g, 5.4 mmol) in anhydrous dichloromethane (12 mL) and anhydrous acetonitrile (18 mL) was added activated 4 Å powdered molecular sieves (5 g) and the reaction mixture was stirred at room temperature for 1 h under nitrogen atmosphere. Then the solution was cooled to −78° C., iodine monobromide (670 mg, 3.24 mmol) dissolved in dichloromethane (4 mL) was added dropwise and the resulting mixture was stirred at same temperature for 2 h. After completion, the reaction mixture was quenched by addition of triethylamine (5 mL) and allowed to stir at room temperature for 1 h. Then the reaction mixture was filtered, filtrate was washed with aqueous sodium thiosulfate solution, dried over sodium sulfate, filtered, and concentrated under reduced pressure. The crude residue was purified by C18 reverse phase column chromatography using water: acetonitrile (+0.05% TFA) as eluents to afford the desired (1R,2R)-1-((2R,3R,4S,6R)-3-acetamido-4-acetoxy-6-((6-(1-(2,2-dimethyl-4-oxo-3,8,11,14,17-pentaoxa-5-azanonadecan-19-yl)-1H-1,2,3-triazol-4-yl)hexyl)oxy)-6-(methoxycarbonyl)tetrahydro-2H-pyran-2-yl)-3-(3-chlorobenzamido)propane-1,2-diyl diacetate (5) as off-white solid, Yield: 705 mg, 30.80%. LCMS (ESI±) m/z 1057.8 [M+H]⁺.

### Synthesis of (2R,4S,5R,6R)-5-acetamido-6-((1R,2R)-3-(3-chlorobenzamido)-1,2-dihydroxypropyl)-2-((6-(1-(2,2-dimethyl-4-oxo-3,8,11,14,17-pentaoxa-5-azanonadecan-19-yl)-1H-1,2,3-triazol-4-yl)hexyl)oxy)-4-hydroxytetrahydro-2H-pyran-2-carboxylic acid (6)

To a stirred solution of (1R,2R)-1-((2R,3R,4S,6R)-3-acetamido-4-acetoxy-6-((6-(1-(2,2-dimethyl-4-oxo-3,8,11,14,17-pentaoxa-5-azanonadecan-19-yl)-1H-1,2,3-triazol-4-yl)hexyl)oxy)-6-(methoxycarbonyl)tetrahydro-2H-pyran-2-yl)-3-(3-chlorobenzamido)propane-1,2-diyl diacetate (5) (705 mg, 0.67 mmol) in methanol (7 mL) and water (3 mL) was added 25% sodium methoxide in methanol (0.2 mL, 3.33 mmol) at 0° C. and the reaction mixture was stirred at room temperature for 16 h. After completion, the reaction mixture was treated with acidic (Dowex 50, H⁺) to make the pH˜6 and the suspension was filtered, filtrate was concentrated to (2R,4S,5R,6R)-5-acetamido-6-((1R,2R)-3-(3-chlorobenzamido)-1,2-dihydroxypropyl)-2-((6-(1-(2,2-dimethyl-4-oxo-3,8,11,14,17-pentaoxa-5-azanonadecan-19-yl)-1H-1,2,3-triazol-4-yl)hexyl)oxy)-4-hydroxytetrahydro-2H-pyran-2-carboxylic acid (6) as off-white solid, Yield: 470 mg, 76.85%. LCMS (ESI±) m/z 917.7 [M+H]⁺.

### Synthesis of (2R,4S,5R,6R)-5-acetamido-2-((6-(1-(14-amino-3,6,9,12-tetraoxatetradecyl)-1H-1,2,3-triazol-4-yl)hexyl)oxy)-6-((1R,2R)-3-(3-chlorobenzamido)-1,2-dihydroxypropyl)-4-hydroxytetrahydro-2H-pyran-2-carboxylic acid (7)

To a stirred solution of (2R,4S,5R,6R)-5-acetamido-6-((1R,2R)-3-(3-chlorobenzamido)-1,2-dihydroxypropyl)-2-((6-(1-(2,2-dimethyl-4-oxo-3,8,11,14,17-pentaoxa-5-azanonadecan-19-yl)-1H-1,2,3-triazol-4-yl)hexyl)oxy)-4-hydroxytetrahydro-2H-pyran-2-carboxylic acid (6) (0.47 g, 0.51 mmol) in anhydrous dichloromethane (4.7 mL) was added trifluoro acetic acid (1 mL) at 0° C. The reaction mixture was stirred at room temperature for 2 h. After completion, the reaction mixture was concentrated to get the crude which was dissolved in methanol and neutralized with Tetra alkyl ammonium carbonate, the polymer-bound resin. Then the reaction mixture was filtered and filtrate was concentrated to afford (2R,4S,5R,6R)-5-acetamido-2-((6-(1-(14-amino-3,6,9,12-tetraoxatetradecyl)-1H-1,2,3-triazol-4-yl)hexyl)oxy)-6-((1R,2R)-3-(3-chlorobenzamido)-1,2-dihydroxypropyl)-4-hydroxytetrahydro-2H-pyran-2-carboxylic acid (7) as off-White solid, Yield-260 mg, 62.10%. LCMS (ESI±) m/z 817.6 [M+H]⁺.

### Synthesis of ((2R,4S,5R,6R)-5-acetamido-6-((1R,2R)-3-(3-chlorobenzamido)-1,2-dihydroxypropyl)-4-hydroxy-2-((6-(1-(2-oxo-6,9,12,15-tetraoxa-3-azaheptadecan-17-yl)-1H-1,2,3-triazol-4-yl)hexyl)oxy)tetrahydro-2H-pyran-2-carboxylic acid (272)

To a stirred solution of (2R,4S,5R,6R)-5-acetamido-2-((6-(1-(14-amino-3,6,9,12-tetraoxatetradecyl)-1H-1,2,3-triazol-4-yl)hexyl)oxy)-6-((1R,2R)-3-(3-chlorobenzamido)-1,2-dihydroxypropyl)-4-hydroxytetrahydro-2H-pyran-2-carboxylic acid (7) (100 mg, 0.12 mmol) in DMF (1 mL) were added 2,5-dioxopyrrolidin-1-yl acetate (8) (19 mg, 0.12 mmol) at 0° C. The resulting reaction mixture was stirred at room temperature for 1 h. After completion, the reaction mixture was concentrated and purified by prep-HPLC purification by using acetonitrile and water (+0.05%, TFA) to afford the desired product ((2R,4S,5R,6R)-5-acetamido-6-((1R,2R)-3-(3-chlorobenzamido)-1,2-dihydroxypropyl)-4-hydroxy-2-((6-(1-(2-oxo-6,9,12,15-tetraoxa-3-azaheptadecan-17-yl)-1H-1,2,3-triazol-4-yl)hexyl)oxy)tetrahydro-2H-pyran-2-carboxylic acid (272) as a white solid, Yield: 20 mg, 19.02%. LCMS (ESI+) m/z 859.7 [M+H]⁺. 1H NMR (400 MHz, MeOD): δ 7.87 (s, 2H), 7.77 (dt, J=7.6, 1.2 Hz, 1H), 7.55 (dt, J=8.0, 1.2 Hz, 1H), δ 7.46 (t, 1H), 4.59-4.57 (m, 2H), 4.07-4.01 (m, 2H), 3.90 (t, 2H), 3.83-3.76 (m, 5H), 3.71-3.52 (m, 17H), 3.48-3.42 (m, 3H), 3.36-3.21 (m, 4H), 2.72 (m, 3H), 2.01 (s, 3H), 1.95 (s, 3H), 1.74-1.67 (m, 3H), 1.55-1.51 (m, 2H), 1.40-1.52 (m, 3H).

### (2R,4S,5R,6R)-5-acetamido-6-((1R,2R)-3-(3-chlorobenzamido)-1,2-dihydroxypropyl)-2-((6-((3,4-dioxo-2-((2-oxo-6,9,12,15-tetraoxa-3-azaheptadecan-17-yl)amino) cyclobut-1-en-1-yl) amino)hexyl)oxy)-4-hydroxytetrahydro-2H-pyran-2-carboxylic acid (Cpd. 273)

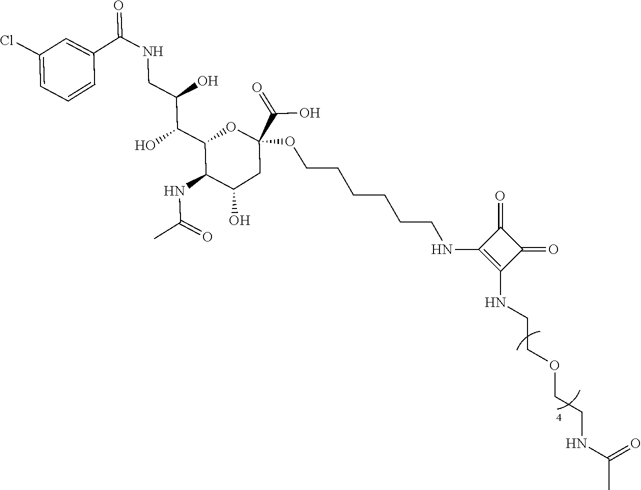

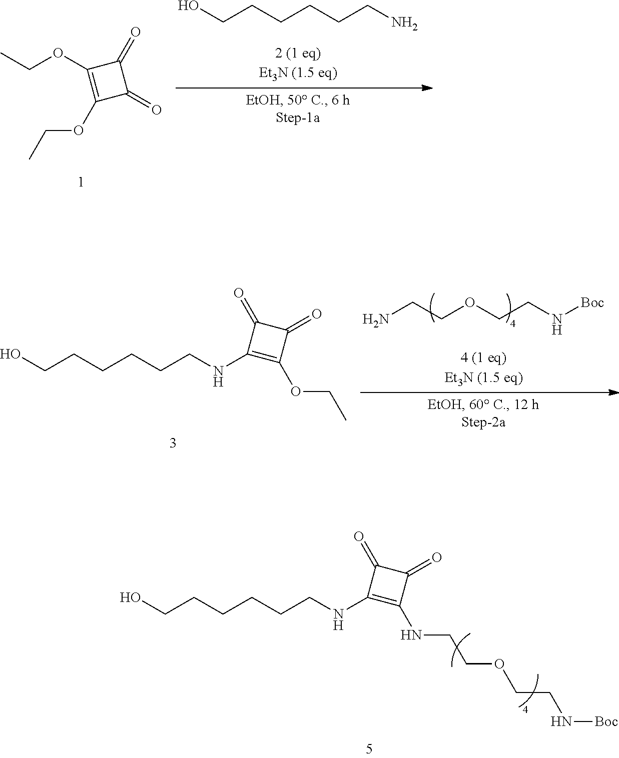

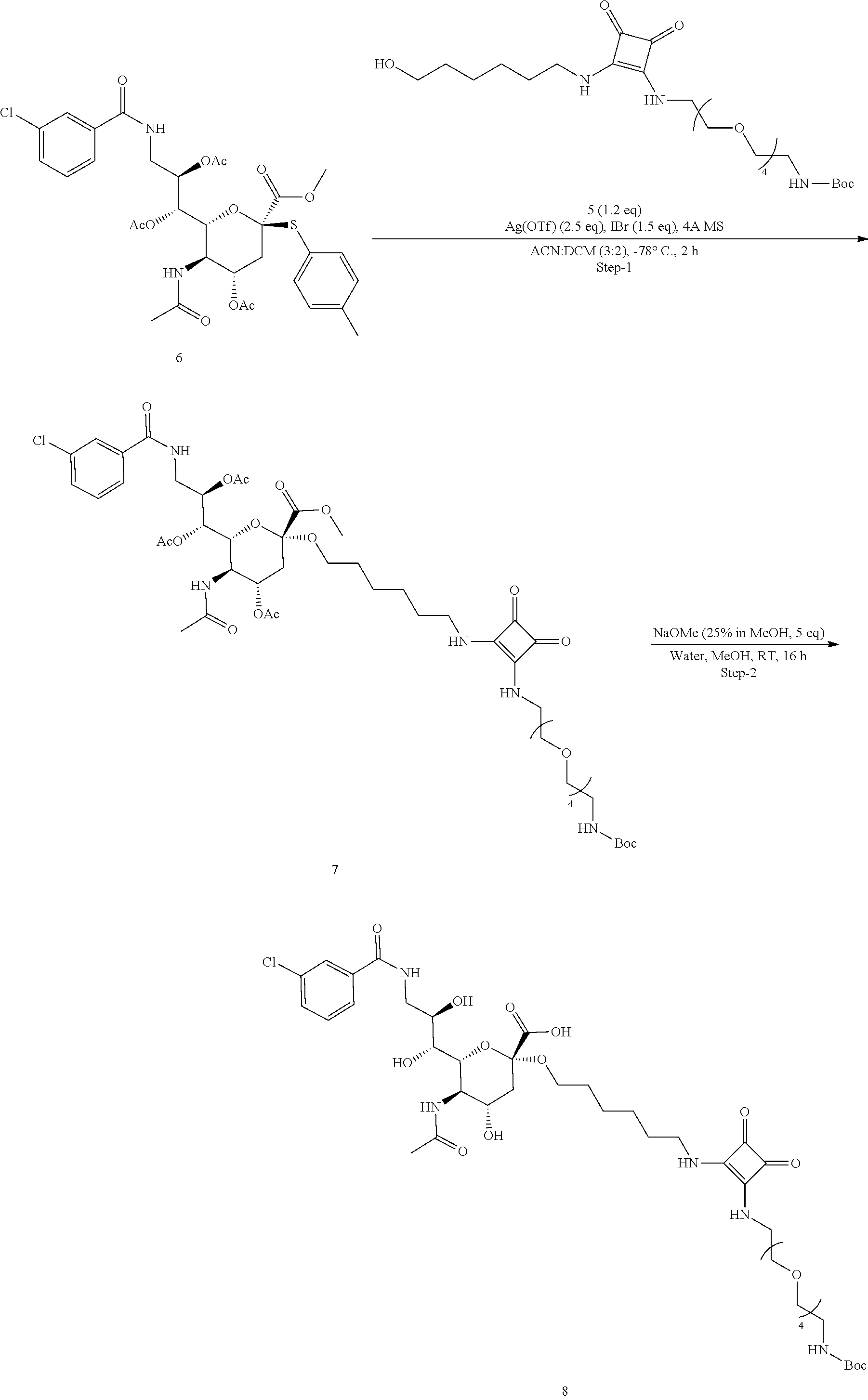

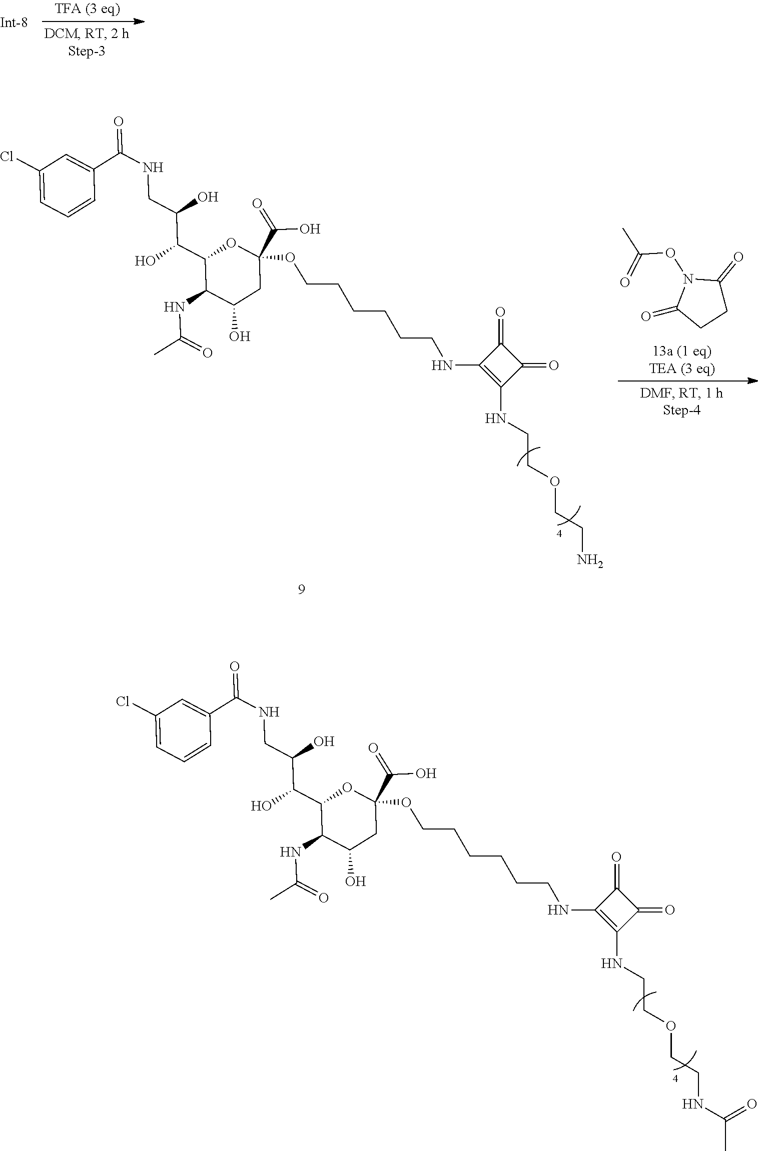

### Synthesis of 3-ethoxy-4-((6-hydroxyhexyl)amino) cyclobut-3-ene-1,2-dione (3)

To a stirred solution of 3,4-diethoxycyclobut-3-ene-1,2-dione (1) (5 g, 29.41 mmol) in Ethanol (50 mL) was added 6-aminohexan-1-ol (2) (1.72 g, 14.7 mmol) followed by addition of triethylamine (6.36 ml, 44.11 mmol) and the resulting reaction mixture was stirred at 50° C. for next 6 h. After completion, the reaction mixture was concentrated to get the crude material, which was purified by Silica gel column chromatography by using Ethyl acetate and Hexane as eluents to afford 3-ethoxy-4-((6-hydroxyhexyl)amino) cyclobut-3-ene-1,2-dione (3) as yellow semi solid, Yield: 5 g, 70.62%. LCMS (ESI±) m/z 242.2 [M+H]⁺.

### Synthesis of tert-butyl (14-((2-((6-hydroxyhexyl)amino)-3,4-dioxocyclobut-1-en-1-yl)amino)-3,6,9,12-tetraoxatetradecyl) carbamate (5)

To a stirred solution of 3-ethoxy-4-((6-hydroxyhexyl)amino)cyclobut-3-ene-1,2-dione (3) (5 g, 20.72 mmol) in Ethanol (50 mL) were added triethylamine (4.49 ml, 31.12 mmol) and tert-butyl (14-amino-3,6,9,12-tetraoxatetradecyl)carbamate (4) (6.97 g, 20.72 mmol) and the resulting reaction mixture was stirred at 60° C. for 12 h. After completion, the reaction mixture was concentrated to get the crude material, which was purified by Silica gel column chromatography, using Ethyl acetate and Hexane as eluents to afford tert-butyl (14-((2-((6-hydroxyhexyl)amino)-3,4-dioxocyclobut-1-en-1-yl)amino)-3,6,9,12-tetraoxatetradecyl) carbamate (5) as white solid, Yield: 3 g, 27.27%. LCMS (ESI±) m/z 532.4 [M+H]⁺.

### Synthesis of (1R,2R)-1-((2R,3R,4S,6R)-3-acetamido-4-acetoxy-6-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-6-(methoxycarbonyl)tetrahydro-2H-pyran-2-yl)-3-(3-chlorobenzamido)propane-1,2-diyl diacetate (7)

To a stirred solution of (1R,2R)-1-((2R,3R,4S,6R)-3-acetamido-4-acetoxy-6-(methoxycarbonyl)-6-(p-tolylthio)tetrahydro-2H-pyran-2-yl)-3-(3-chlorobenzamido)propane-1,2-diyl diacetate (6) (1.5 g, 2.16 mmol) tert-butyl (14-((2-((6-hydroxyhexyl)amino)-3,4-dioxocyclobut-1-en-1-yl)amino)-3,6,9,12-tetraoxatetradecyl) carbamate (5) (1.38 g, 2.60 mmol) and Silver (1+) trifluoro methane sulfonate (AgOTf) (1.39 g, 5.4 mmol) in anhydrous dichloromethane (12 mL) and anhydrous acetonitrile (18 mL) was added activated 4 Å powdered molecular sieves (5 g) and the reaction mixture was stirred at room temperature for 1 h under nitrogen atmosphere. Then the solution was cooled to −78° C., iodine monobromide (670 mg, 3.24 mmol) dissolved in dichloromethane (4 mL) was added dropwise, and the resulting mixture was stirred at same temperature for 2 h. After completion, the reaction mixture was quenched by addition of triethylamine (5 mL) and allowed to stir at room temperature for 1 h. Then the reaction mixture was filtered, the filtrate was washed with aqueous sodium thiosulfate solution, dried over sodium sulfate, filtered, and concentrated under reduced pressure. The crude residue obtained was purified by C18 reverse phase column chromatography using water: acetonitrile (+0.05% TFA) as eluents to afford the desired (1R,2R)-1-((2R,3R,4S,6R)-3-acetamido-4-acetoxy-6-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-6-(methoxycarbonyl)tetrahydro-2H-pyran-2-yl)-3-(3-chlorobenzamido)propane-1,2-diyl diacetate (7) as pale yellow solid, Yield: 700 mg, 29.39%; LCMS (ESI±) m/z 1101.0 [M+H]⁺.

### Synthesis of (2R,4S,5R,6R)-5-acetamido-6-((1R,2R)-3-(3-chlorobenzamido)-1,2-dihydroxypropyl)-2-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-4-hydroxytetrahydro-2H-pyran-2-carboxylic acid (8)

To a stirred solution of (1R,2R)-1-((2R,3R,4S,6R)-3-acetamido-4-acetoxy-6-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-6-(methoxycarbonyl)tetrahydro-2H-pyran-2-yl)-3-(3-chlorobenzamido)propane-1,2-diyl diacetate (7) (700 mg, 0.64 mmol) in methanol (7 mL) and water (3 mL) was added 25% sodium methoxide in methanol (0.2 mL, 3.18 mmol) at 0° C. and the reaction mixture was stirred at room temperature for 16 h. After completion the reaction mixture was treated with acidic (Dowex 50, H⁺) to make the pH˜6 and the suspension was filtered, filtrate was concentrated to afford (2R,4S,5R,6R)-5-acetamido-6-((1R,2R)-3-(3-chlorobenzamido)-1,2-dihydroxypropyl)-2-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-4-hydroxytetrahydro-2H-pyran-2-carboxylic acid (8) as pale yellow solid, Yield: 450 mg, 73.67%. LCMS (ESI±) m/z 958.6 [M−H]⁻.

### Synthesis of (2R,4S,5R,6R)-5-acetamido-2-((6-((2-((14-amino-3,6,9,12-tetraoxatetradecyl) amino)-3,4-dioxocyclobut-1-en-1-yl)amino)hexyl)oxy)-6-((1R,2R)-3-(3-chlorobenzamido)-1,2-dihydroxypropyl)-4-hydroxytetrahydro-2H-pyran-2-carboxylic acid (9)

To a stirred solution of (2R,4S,5R,6R)-5-acetamido-6-((1R,2R)-3-(3-chlorobenzamido)-1,2-dihydroxypropyl)-2-((6-((2-((2,2-dimethyl-4-oxo-3,8,11,14,17-pentaoxa-5-azanonadecan-19-yl)amino)-3,4-dioxocyclobut-1-en-1-yl)amino)hexyl)oxy)-4-hydroxytetrahydro-2H-pyran-2-carboxylic acid (8) (450 mg, 0.52 mmol) in anhydrous dichloromethane (4.5 mL) was added trifluoro acetic acid (1 mL) at 0° C. The reaction mixture was stirred at room temperature for 2 h. After completion, the reaction mixture was concentrated to get the crude which was dissolved in methanol and neutralized with Tetra alkyl ammonium carbonate, the polymer-bound resin. Then the reaction mixture was filtered and filtrate was concentrated to afford (2R,4S,5R,6R)-5-acetamido-2-((6-((2-((14-amino-3,6,9,12-tetraoxatetradecyl)amino)-3,4-dioxocyclobut-1-en-1-yl)amino)hexyl)oxy)-6-((1R,2R)-3-(3-chlorobenzamido)-1,2-dihydroxypropyl)-4-hydroxytetrahydro-2H-pyran-2-carboxylic acid (9) as off-white solid, Yield-290 mg, 71.94%. LCMS (ESI±) m/z 858.7 [M−H]⁻.

### Synthesis of (2R,4S,5R,6R)-5-acetamido-6-((1R,2R)-3-(3-chlorobenzamido)-1,2-dihydroxypropyl)-2-((6-((3,4-dioxo-2-((2-oxo-6,9,12,15-tetraoxa-3-azaheptadecan-17-yl)amino)cyclobut-1-en-1-yl)amino)hexyl)oxy)-4-hydroxytetrahydro-2H-pyran-2-carboxylic acid (273)

To a stirred solution of (2R,4S,5R,6R)-5-acetamido-2-((6-((2-((14-amino-3,6,9,12-tetraoxatetradecyl)amino)-3,4-dioxocyclobut-1-en-1-yl)amino)hexyl)oxy)-6-((1R,2R)-3-(3-chlorobenzamido)-1,2-dihydroxypropyl)-4-hydroxytetrahydro-2H-pyran-2-carboxylic acid (9) (100 mg, 0.12 mmol) in DMF (1 mL) were added 2,5-dioxopyrrolidin-1-yl acetate (9) (19 mg, 0.12 mmol) at 0° C. The resulting reaction mixture was stirred at room temperature for 1 h. After completion, the reaction mixture was concentrated and purified by prep-HPLC purification by using acetonitrile and water (+0.05%, TFA) to afford the desired product (2R,4S,5R,6R)-5-acetamido-6-((1R,2R)-3-(3-chlorobenzamido)-1,2-dihydroxypropyl)-2-((6-((3,4-dioxo-2-((2-oxo-6,9,12,15-tetraoxa-3-azaheptadecan-17-yl)amino)cyclobut-1-en-1-yl)amino)hexyl)oxy)-4-hydroxytetrahydro-2H-pyran-2-carboxylic acid (273) as a white solid, Yield: 40 mg, 38.14%. LCMS (ESI+) m/z 902.6 [M+H]⁺. ¹H NMR (400 MHz, MeOD): δ 7.87 (t, 1H), 7.79 (dt, J=7.6, 1.2 Hz, 1H), 7.77 (dt, J=8.0, 1.2 Hz, 1H), δ 7.47 (t, 1H), 4.06-4.01 (m, 1H), 3.85-3.71 (m, 7H), 3.70-3.61 (m, 15H), 3.55 (t, 3H), 3.48-3.27 (m, 3H), 2.73 (dd, J=12.8, 4.8 Hz, 1H), δ 2.01 (s, 3H), 1.96 (s, 3H), 1.74 (t, J=24.4 Hz, 1H), 1.59 (q, J=20.0 Hz, 4H), 1.40 (q, J=7.5 Hz, 4H).

### (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-((1-(15-oxo-15-(perfluorophenoxy)-3,6,9,12-tetraoxapentadecyl)-1H-1,2,3-triazol-4-yl)methoxy)ethoxy)ethoxy)tetrahydro-2H-pyran-2-carboxylic acid (Cpd. 41P)

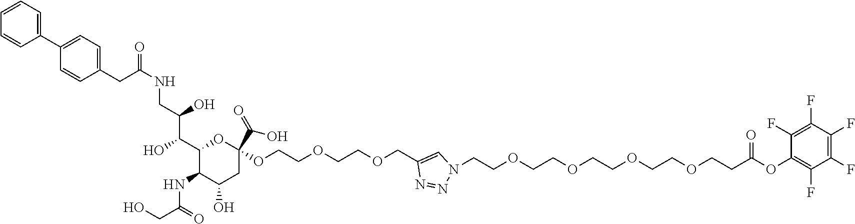 [SMILES: O=C(Cc1ccc(-c2ccccc2)cc1)NC[C@@H](O)[C@@H](O)[C@@H]1O[C@@](OCCOCCOCc2cn(CCOCCOCCOCCOCCC(=O)Oc3c(F)c(F)c(F)c(F)c3F)nn2)(C(=O)O)C[C@H](O)[C@H]1NC(=O)CO]

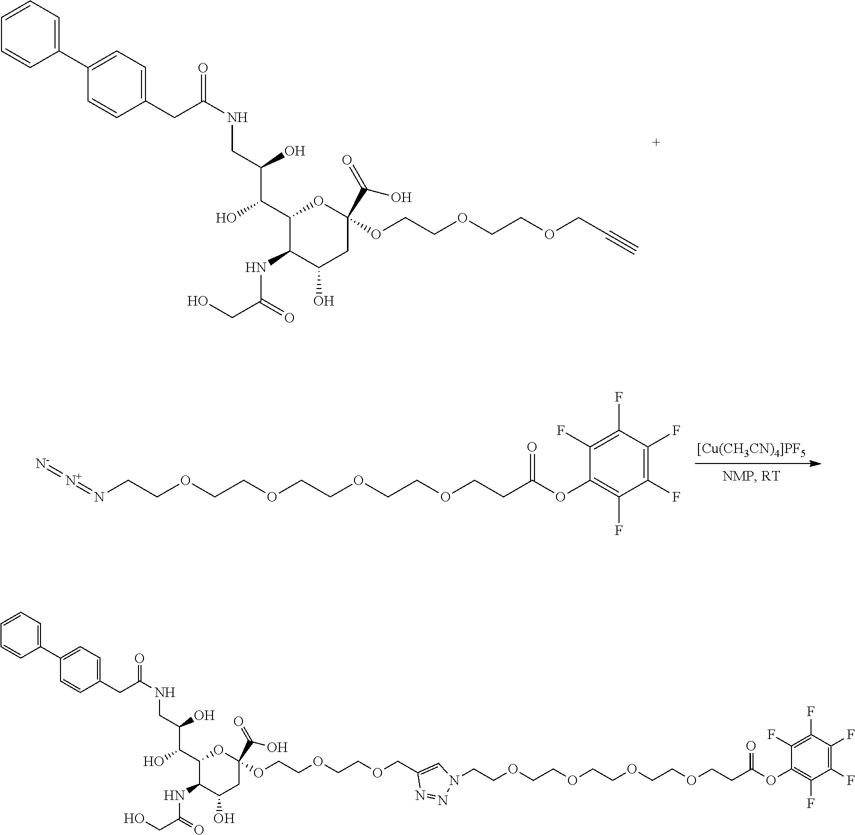

### Synthesis of (2R,4S,5R,6R)-6-((1R,2R)-3-(2-([1,1′-biphenyl]-4-yl)acetamido)-1,2-dihydroxy propyl)-4-hydroxy-5-(2-hydroxyacetamido)-2-(2-(2-((1-(15-oxo-15-(perfluorophenoxy)-3,6,9,12-tetraoxapentadecyl)-1H-1,2,3-triazol-4-yl)methoxy)ethoxy)ethoxy)tetrahydro-2H-pyran-2-carboxylic acid (41P)

To a solution of (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-(prop-2-yn-1-yloxy)ethoxy)ethoxy)tetrahydro-2H-pyran-2-carboxylic acid (110, 150.0 mg, 233 umol) in anhydrous NMP (1.50 mL) was added Azido-PEG4-PFP ester (128 mg, 279 umol) in anhydrous NMP (1.50 mL) dropwise, followed by Tetrakis(acetonitrile)copper(l) hexafluorophosphate (130 mg, 349 umol). The vial was sealed, and the mixture was stirred for 45 min at room temperature. The mixture was filtered through a 0.2 μm PTFE filter, a few drops of acetic acid were added, and the filtrate was purified by C18 reverse phase column chromatography using acetonitrile and water (+0.1% TFA) as eluents. The fractions containing the desired product were lyophilized to afford 41P as a white solid. Yield: 164 mg, 64%; LCMS (ESI) m/z 1102.0 [M+H]⁺. ¹H NMR (500 MHz, Methanol-d₄): δ 8.02 (s, 1H), 1.87 (m, 1H), 7.60-7.56 (m, 4H), 7.41-7.37 (m, 4H), 7.31 (t, J=7.5 Hz, 1H), 4.62 (s, 2H), 4.56 (t, J=5.0 Hz, 2H), 4.01 (s, 2H), 3.90-3.81 (m, 8H), 3.73-3.56 (m, 23H), 3.38 (dd, J=8.5 Hz & 1.5 Hz, 1H), 3.25 (dd, J=14.0 Hz, 8.0 Hz, 1H), 2.96 (t, J=6.0 Hz, 2H), 2.72 (dd, J=12.5 Hz & 4.5 Hz, 1H), 1.74 (t, J=12.0 Hz, 1H).

### (2R,4S,5R,6R)-6-((1R,2R)-3-(2-([1,1′-biphenyl]-4-yl)acetamido)-1,2-dihydroxypropyl)-2-(2-(2-((1-(18-(2,5-dioxo-2,5-dihydro-1H-pyrrol-1-yl)-15-oxo-3,6,9,12-tetraoxa-16-azaoctadecyl)-1H-1,2,3-triazol-4-yl)methoxy)ethoxy)ethoxy)-4-hydroxy-5-(2-hydroxyacetamido)tetrahydro-2H-pyran-2-carboxylic acid (Cpd. 41M)

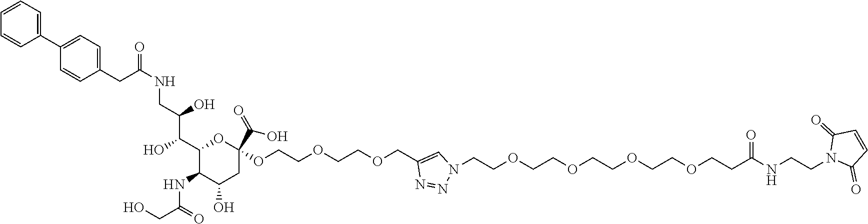 [SMILES: O=C(CCOCCOCCOCCOCCn1cc(COCCOCCO[C@]2(C(=O)O)C[C@H](O)[C@@H](NC(=O)CO)[C@H]([C@H](O)[C@H](O)CNC(=O)Cc3ccc(-c4ccccc4)cc3)O2)nn1)NCCN1C(=O)C=CC1=O]

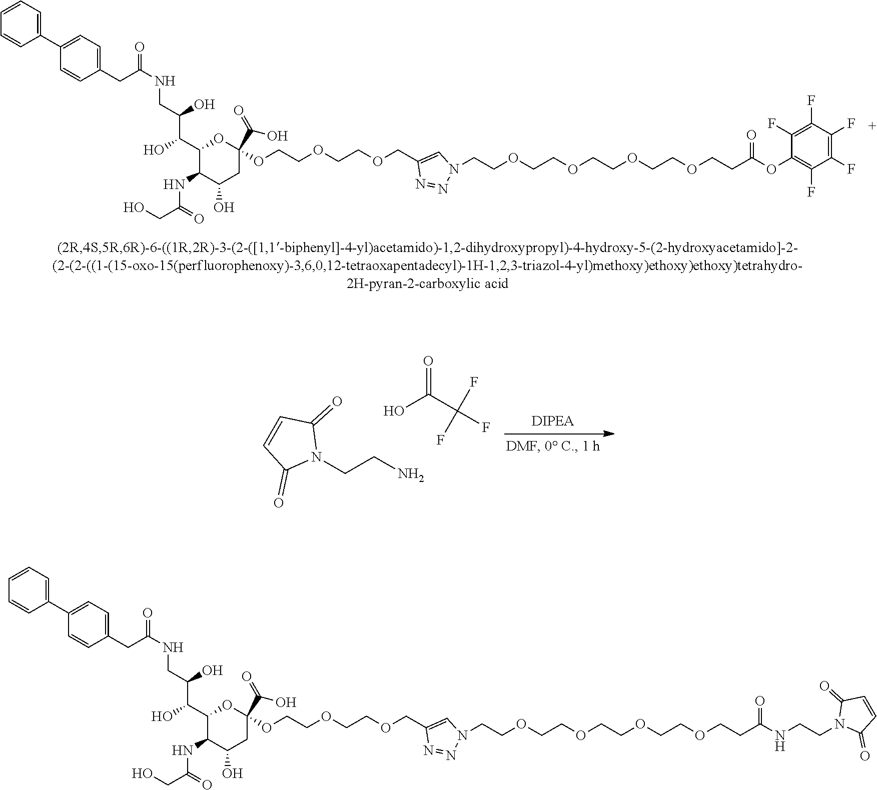 [SMILES: O=C(Cc1ccc(-c2ccccc2)cc1)NC[C@@H](O)[C@@H](O)[C@@H]1O[C@@](OCCOCCOCc2cn(CCOCCOCCOCCOCCC(=O)Oc3c(F)c(F)c(F)c(F)c3F)nn2)(C(=O)O)C[C@H](O)[C@H]1NC(=O)CO ; NCCN1C(=O)C=CC1=O ; O=C(O)C(F)(F)F ; O=C(CCOCCOCCOCCOCCn1cc(COCCOCCO[C@]2(C(=O)O)C[C@H](O)[C@@H](NC(=O)CO)[C@H]([C@H](O)[C@H](O)CNC(=O)Cc3ccc(-c4ccccc4)cc3)O2)nn1)NCCN1C(=O)C=CC1=O]

### Synthesis of (2R,4S,5R,6R)-6-((1R,2R)-3-(2-([1,1′-biphenyl]-4-yl)acetamido)-1,2-dihydroxypropyl)-2-(2-(2-((1-(18-(2,5-dioxo-2,5-dihydro-1H-pyrrol-1-yl)-15-oxo-3,6,9,12-tetraoxa-16-azaoctadecyl)-1H-1,2,3-triazol-4-yl)methoxy)ethoxy)ethoxy)-4-hydroxy-5-(2-hydroxyacetamido)tetrahydro-2H-pyran-2-carboxylic acid (41M)

To a solution of (2R,4S,5R,6R)-6-((1R,2R)-3-(2-([1,1′-biphenyl]-4-yl)acetamido)-1,2-dihydroxypropyl)-4-hydroxy-5-(2-hydroxyacetamido)-2-(2-(2-((1-(15-oxo-15-(perfluorophenoxy)-3,6,9,12-tetraoxapentadecyl)-1H-1,2,3-triazol-4-yl)methoxy)ethoxy)ethoxy)tetrahydro-2H-pyran-2-carboxylic acid (41P, 76.4 mg, 69.3 umol) in DMF (1.73 mL) was added n-(2-aminoethyl)maleimide trifluoroacetate salt (18.5 mg, 72.8 umol). The mixture was cooled down to 0° C. and stirred for 10 min. DIEA (36.2 uL, 208 umol) was added and the mixture was stirred for 1 h at 0° C. The reaction mixture was filtered and purified by preparatory HPLC using acetonitrile and water (+0.05% TFA) as eluents. The fractions containing the desired product were combined and lyophilized to dryness to afford 41M as a white solid. Yield: 59.8 mg, 82%; LCMS (ESI) m/z 1058 [M+H]⁺. ¹H NMR (400 MHz, MeOD) δ 8.05 (d, J=7.1 Hz, 1H), 7.64-7.55 (m, 4H), 6.81 (s, 1H), 4.66-4.54 (m, 4H), 4.03 (s, 2H), 3.90 (q, J=5.6 Hz, 4H), 3.86-3.77 (m, 1H), 3.74 (d, J=9.5 Hz, 1H), 3.73-3.56 (m, 26H), 3.43-3.33 (m, 4H), 3.26 (dd, J=13.8, 7.6 Hz, 1H), 2.78-2.70 (m, 1H), 2.40 (dt, J=29.8, 6.0 Hz, 2H), 1.74 (t, J=11.8 Hz, 1H), 1.30 (s, 1H).

### (2R,4S,5R,6R)-6-((1R,2R)-1,2-dihydroxy-3-(2-(4′-hydroxy-[1,1′-biphenyl]-4-yl)acetamido)propyl)-4-hydroxy-5-(2-hydroxyacetamido)-2-(4-(4-((1-(15-oxo-15-(perfluorophenoxy)-3,6,9,12-tetraoxapentadecyl)-1H-1,2,3-triazol-4-yl)methoxy)phenyl)-1H-1,2,3-triazol-1-yl)tetrahydro-2H-pyran-2-carboxylic acid (Cpd. 53P)

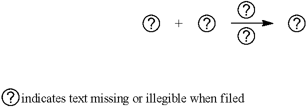

### Synthesis of (2R,4S,5R,6R)-6-((1R,2R)-1,2-dihydroxy-3-(2-(4′-hydroxy-[1,1′-biphenyl]-4-yl)acetamido)propyl)-4-hydroxy-5-(2-hydroxyacetamido)-2-(4-(4-((1-(15-oxo-15-(perfluorophenoxy)-3,6,9,12-tetraoxapentadecyl)-1H-1,2,3-triazol-4-yl)methoxy)phenyl)-1H-1,2,3-triazol-1-yl)tetrahydro-2H-pyran-2-carboxylic acid (53P)

To a solution of (2R,4S,5R,6R)-6-((1R,2R)-1,2-dihydroxy-3-(2-(4′-hydroxy-[1,1′-biphenyl]-4-yl)acetamido)propyl)-4-hydroxy-5-(2-hydroxyacetamido)-2-(4-(4-(prop-2-yn-1-yloxy)phenyl)-1H-1,2,3-triazol-1-yl)tetrahydro-2H-pyran-2-carboxylic acid (30.0 mg, 41.9 umol) in anhydrous NMP (270 uL) was added Azido-PEG4-PFP ester (22.0 mg, 48.2 umol) in anhydrous NMP (270 uL) dropwise, followed by Tetrakis(acetonitrile)copper(l) hexafluorophosphate (23.4 mg, 62.9 umol). The vial was sealed, and the mixture was stirred for 45 min at room temperature. The mixture was filtered through a 0.2 μm PTFE filter, a few drops of acetic acid were added, and the filtrate was purified by C18 reverse phase column chromatography using acetonitrile and water (+0.1% TFA) as eluents. The fractions containing the desired product were lyophilized to afford Cpd. 53P as a white solid. Yield: 32.0 mg, 65%; LCMS (ESI) m/z 1171.8 [M−H]⁻. ¹H NMR (500 MHz, DMSO-d₆): 9.48 (s, br, 1H), 8.82 (s, 1H), 8.19 (s, 1H), 7.94 (m, br, 1H), 7.93 (m, br, 1H), 7.84 (d, J=8.5 Hz, 2H), 7.47 (d, J=8.5 Hz, 2H), 7.43 (d, J=8.5 Hz, 2H), 7.28 (d, J=8.5 Hz, 2H), 7.10 (d, J=8.5 Hz, 2H), 6.81 (d, J=8.5 Hz, 2H), 5.16 (s, 2H), 4.54 (t, J=5.5 Hz, 2H), 4.12-4.07 (m, 1H), 4.02-4.00 (m, 1H), 3.89 (m, 2H), 3.83-3.80 (m, 3H), 3.75-3.71 (m, 3H), 3.57 -3.55 (m, 1H), 3.53-3.51 (m, 4H), 3.49-3.45 (m, 12H), 3.09 (m, 1H), 3.00 (t, J=6.0 Hz, 2H), 2.16 (m, 1H).

### (2S,4S,5R,6R)-6-((1R,2R)-3-(2-(4-chlorophenyl)acetamido)-1,2-dihydroxypropyl)-4-hydroxy-2-(1-hydroxy-7-((1-(15-oxo-15-(perfluorophenoxy)-3,6,9,12-tetraoxapentadecyl)-1H-1,2,3-triazol-4-yl)methoxy)heptyl)-5-(2-hydroxyacetamido)tetrahydro-2H-pyran-2-carboxylic acid (Cpd. 103P)

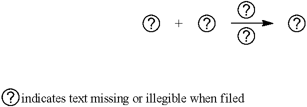

### Synthesis of (2S,4S,5R,6R)-6-((1R,2R)-3-(2-(4-chlorophenyl)acetamido)-1,2-dihydroxypropyl)-4-hydroxy-2-(1-hydroxy-7-((1-(15-oxo-15-(perfluorophenoxy)-3,6,9,12-tetraoxapentadecyl)-1H-1,2,3-triazol-4-yl)methoxy)heptyl)-5-(2-hydroxyacetamido)tetrahydro-2H-pyran-2-carboxylic acid (Cpd. 103P)

To a solution of (2S,4S,5R,6R)-6-((1R,2R)-3-(2-(4-chlorophenyl)acetamido)-1,2-dihydroxypropyl)-4-hydroxy-2-(1-hydroxy-7-(prop-2-yn-1-yloxy)heptyl)-5-(2-hydroxyacetamido)tetrahydro-2H-pyran-2-carboxylic acid (14.7 mg, 23.4 umol) in anhydrous NMP (150 uL) was added Azido-PEG4-PFP ester (12.0 mg, 26.2 umol) in anhydrous NMP (150 uL) dropwise, followed by Tetrakis(acetonitrile)copper(l) hexafluorophosphate (13.1 mg, 35.1 umol). The vial was sealed, and the mixture was stirred for 45 min at room temperature. The mixture was filtered through a 0.2 μm PTFE filter, a few drops of acetic acid were added, and the filtrate was purified by C18 reverse phase column chromatography using acetonitrile and water (+0.1% TFA) as eluents. The fractions containing the desired product were lyophilized to afford Cpd. 103P as a white solid. Yield: 16.6 mg, 65%; LCMS (ESI) m/z 1086.0 [M+H]⁺. ¹H NMR (500 MHz, DMSO-d₆): 8.02 (s, br, 1H), 8.00 (m, br, 1H), 7.65 (m, br, 1H), 7.32 (d, J=9.0 Hz, 2H), 7.27 (d, J=9.0 Hz, 2H), 5.41 (m, 1H), 4.77 (m, 1H), 4.54-4.48 (m, 3H), 4.46 (s, 2H), 3.83 (m, 2H), 3.80 (t, J=5.5 Hz, 2H), 3.76 (t, J=6.0 Hz, 2H), 3.67-3.57 (m, 3H), 3.55-3.44 (m, 16H), 3.42-3.39 (m, 2H), 3.15-3.11 (m, 1H), 3.01 (t, J=6.0 Hz, 2H), 2.88-2.83 (m, 1H), 1.74-1.68 (m, 1H), 1.49-1.45 (m, 4H), 1.26-1.19 (m, br, 6H).

### (2R,4S,5R,6R)-6-((1R,2R)-3-(2-(4-chlorophenyl)acetamido)-1,2-dihydroxypropyl)-4-hydroxy-5-(2-hydroxyacetamido)-2-((14-oxo-1-(1-(15-oxo-15-(perfluorophenoxy)-3,6,9,12-tetraoxapentadecyl)-1H-1,2,3-triazol-4-yl)-2,5,8,11,18,21-hexaoxa-15-azatricosan-23-yl)oxy)tetrahydro-2H-pyran-2-carboxylic acid (Cpd. 111P)

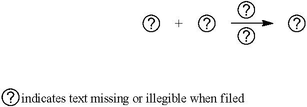

### Synthesis of (2R,4S,5R,6R)-6-((1R,2R)-3-(2-(4-chlorophenyl)acetamido)-1,2-dihydroxypropyl)-4-hydroxy-5-(2-hydroxyacetamido)-2-((14-oxo-1-(1-(15-oxo-15-(perfluorophenoxy)-3,6,9,12-tetraoxapentadecyl)-1H-1,2,3-triazol-4-yl)-2,5,8,11,18,21-hexaoxa-15-azatricosan-23-yl)oxy)tetrahydro-2H-pyran-2-carboxylic acid

To a solution of Azido-PEG4-PFP-ester (12.6 mg, 27.5 umol) in anhydrous NMP (162 uL) was added (2R,4S,5R,6R)-6-((1R,2R)-3-(2-(4-chlorophenyl)acetamido)-1,2-dihydroxypropyl)-4-hydroxy-5-(2-hydroxyacetamido)-2-((10-oxo-3,6,13,16,19,22-hexaoxa-9-azapentacos-24-yn-1-yl)oxy)tetrahydro-2H-pyran-2-carboxylic acid (Cpd. 111, 20.9 mg, 24.6 umol) in anhydrous NMP (162 uL) dropwise, followed by Tetrakis(acetonitrile)copper(l) hexafluorophosphate (13.7 mg, 36.9 umol). The vial was sealed and the mixture was stirred for 30 min at room temperature. The mixture was filtered through a 0.2 μm PTFE filter, a few drops of acetic acid were added, and the filtrate was purified by C18 reverse phase column chromatography using acetonitrile and water (+0.1% TFA) as eluents. The fractions containing the desired product were lyophilized to afford Cpd. 111P as a colorless solid. Yield: 17.3 mg, 54%; LCMS (ESI) m/z 1307 [M+H]⁺. ¹H NMR (500 MHz, DMSO-d₆): 8.04 (s, 1H), 7.98 (t, J=5.0 Hz, 1H), 7.91 (t, J=5.0 Hz, 1H), 7.83 (d, J=7.5 Hz, 1H), 7.33 (d, J=8.5 Hz, 2H), 7.27 (d, J=8.5 Hz, 2H), 5.52 (t, br, J=5.5 Hz, 1H), 4.95 (d, br, J=5.0 Hz, 1H), 4.71 (d, br, J=5.5 Hz, 1H), 4.51-4.49 (m, 4H), 3.86 (t, J=5.0 Hz, 1H), 3.80 (t, J=5.5 Hz, 2H), 3.76 (t, J=5.5 Hz, 2H), 3.74-3.72 (m, 1H), 3.68-3.64 (m, 1H), 3.57 (t, J=6.5 Hz, 2H), 3.55-3.53 (m, 5H), 3.52-3.44 (m, 32H), 3.39 (t, J=6.0 Hz, 4H), 3.19 (t, J=6.0 Hz, 2H), 3.18 (t, J=6.0 Hz, 2H), 3.01 (t, J=6.0 Hz, 2H), 2.95-2.91 (m, 1H), 2.54-2.51 (m, 1H), 2.30 (t, J=6.5 Hz, 2H).

### (2S,4R,5S,6S)-2-((6-((carboxymethyl)((1-(15-oxo-15-(perfluorophenoxy)-3,6,9,12-tetraoxapentadecyl)-1H-1,2,3-triazol-4-yl)methyl)amino)-6-oxohexyl)oxy)-6-((1S,2S)-3-(2-(4-chlorophenyl)acetamido)-1,2-dihydroxypropyl)-4-hydroxy-5-(2-hydroxyacetamido)tetrahydro-2H-pyran-2-carboxylic acid (Cpd. 100P)

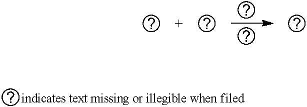

### Synthesis of (2S,4R,5S,6S)-2-((6-((carboxymethyl)((1-(15-oxo-15-(perfluorophenoxy)-3,6,9,12-tetraoxapentadecyl)-1H-1,2,3-triazol-4-yl)methyl)amino)-6-oxohexyl)oxy)-6-((1S,2S)-3-(2-(4-chlorophenyl)acetamido)-1,2-dihydroxypropyl)-4-hydroxy-5-(2-hydroxyacetamido)tetrahydro-2H-pyran-2-carboxylic acid

To a solution of (2S,4R,5S,6S)-2-((6-((carboxymethyl)(prop-2-yn-1-yl)amino)-6-oxohexyl)oxy)-6-((1S,2S)-3-(2-(4-chlorophenyl)acetamido)-1,2-dihydroxypropyl)-4-hydroxy-5-(2-hydroxyacetamido)tetrahydro-2H-pyran-2-carboxylic acid (Cpd. 100, 28.3 mg, 41.2 umol) in anhydrous NMP (550 uL) was added Azido-PEG4-PFP-ester (20.8 mg, 45.4 umol), followed by Tetrakis(acetonitrile)copper(l) hexafluorophosphate (38.4 mg, 103 umol). The vial was sealed and the mixture was stirred for 1 hour at room temperature. The mixture was filtered through a 0.2 μm PTFE filter, a few drops of acetic acid were added, and the filtrate was purified by C18 reverse phase column chromatography using acetonitrile and water (+0.1% TFA) as eluents. The fractions containing the desired product were lyophilized to afford Cpd. 100P as a white solid. Yield: 33.4 mg, 71%; LCMS (ESI) m/z 1143 [M+H]⁺; 1141.5 [M−H]⁻. ¹H NMR (500 MHz, DMSO-d₆): 8.08 & 7.91 (s, 1H), 7.98 (t, J=5.5 Hz, 1H), 7.84 (d, J=7.5 Hz, 1H), 7.32 (d, J=8.5 Hz, 2H), 7.26 (d, J=8.5 Hz, 2H), 5.53 (s, br, 1H), 4.95 (s, br, 1H), 4.59 (s, 1H), 4.50-4.45 (m, 3H), 4.29 (s, br, 1H), 4.10 (s, 1H), 3.92-3.83 (m, 3H), 3.80-3.74 (m, 5H), 3.66-3.60 (m, 2H), 3.55-3.44 (m, 19H), 3.21-3.19 (m, 2H), 3.01 (t, J=6.0 Hz, 2H), 2.98-2.93 (m, 1H), 2.20 (t, J=7.5 Hz, 1H), 1.54-1.43 (m, 5H), 1.30-1.24 (m, 2H).

### (2S,4R,5S,6S)-2-((6-((carboxymethyl)((1-(18-(2,5-dioxo-2,5-dihydro-1H-pyrrol-1-yl)-15-oxo-3,6,9,12-tetraoxa-16-azaoctadecyl)-1H-1,2,3-triazol-4-yl)methyl)amino)-6-oxohexyl)oxy)-6-((1S,2S)-3-(2-(4-chlorophenyl)acetamido)-1,2-dihydroxypropyl)-4-hydroxy-5-(2-hydroxyacetamido)tetrahydro-2H-pyran-2-carboxylic acid (Cpd. 100M)

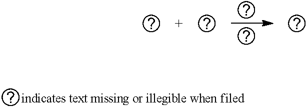

### Synthesis of (2S,4R,5S,6S)-2-((6-((carboxymethyl)((1-(18-(2,5-dioxo-2,5-dihydro-1H-pyrrol-1-yl)-15-oxo-3,6,9,12-tetraoxa-16-azaoctadecyl)-1H-1,2,3-triazol-4-yl)methyl)amino)-6-oxohexyl)oxy)-6-((1S,2S)-3-(2-(4-chlorophenyl)acetamido)-1,2-dihydroxypropyl)-4-hydroxy-5-(2-hydroxyacetamido)tetrahydro-2H-pyran-2-carboxylic acid

To a solution of (2S,4R,5S,6S)-2-((6-((carboxyethl)((1-(15-oxo-15-(perfluorophenoxy)-3,6,9,12-tetraoxapentadecyl)-1H-1,2,3-triazol-4-yl)methyl)amino)-6-oxohexyl)oxy)-6-((1S,2S)-3-(2-(4-chlorophenyl)acetamido)-1,2-dihydroxypropyl)-4-hydroxy-5-(2-hydroxyacetamido)tetrahydro-2H-pyran-2-carboxylic acid (Cpd. 100P, 28.0 mg, 24.5 umol) in DMF (612 uL) was added n-(2-aminoethyl)maleimide trifluoroacetate salt (6.53 mg, 25.7 umol). The mixture was cooled down to 0° C. and stirred for 10 min. DIEA (12.8 uL, 73.5 umol) was added and the mixture was stirred for 1 hr at 0° C. The reaction mixture was filtered and purified by preparatory HPLC using acetonitrile and water (+0.05% TFA) as eluents. The fractions containing the desired product were combined and lyophilized to afford Cpd. 100M as a white solid. Yield: 24.8 mg, 92%; LCMS (ESI) m/z 1099.3 [M+H]⁺. ¹H NMR (500 MHz, DMSO-d₆): 8.08 & 7.91 (s, 1H), 7.98 (t, J=5.5 Hz, 1H), 7.93 (t, J=5.5 Hz, 1H), 7.84 (d, J=7.5 Hz, 1H), 7.32 (d, J=8.5 Hz, 2H), 7.27 (d, J=8.5 Hz, 2H), 4.59 (s, 2H), 4.51-4.46 (m, 3H), 4.10 (s, 1H), 3.92 (s, 1H), 3.90-3.83 (m, 2H), 3.80-3.77 (m, 4H), 3.68-3.60 (m, 3H), 3.56-3.49 (m, 8H), 3.47-3.42 (m, 13H), 3.21-3.16 (m, 5H), 2.98-2.93 (m, 1H), 2.20 (t, J=7.5 Hz, 1H), 1.55-1.44 (m, 5H), 1.31-1.23 (m, 2H).

### (2R,4S,5R,6R)-6-((1R,2R)-1,2-dihydroxy-3-(2-(4-isopropylphenyl)acetamido)propyl)-4-hydroxy-5-(2-hydroxyacetamido)-2-((6-((1-(15-oxo-15-(perfluorophenoxy)-3,6,9,12-tetraoxapentadecyl)-1H-1,2,3-triazol-4-yl)methoxy)hexyl)oxy)tetrahydro-2H-pyran-2-carboxylic acid (Cpd. 159P)

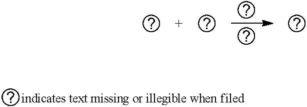

### Synthesis of (2R,4S,5R,6R)-6-((1R,2R)-1,2-dihydroxy-3-(2-(4-isopropylphenyl)acetamido)propyl)-4-hydroxy-5-(2-hydroxyacetamido)-2-((6-((1-(15-oxo-15-(perfluorophenoxy)-3,6,9,12-tetraoxapentadecyl)-1H-1,2,3-triazol-4-yl)methoxy)hexyl)oxy)tetrahydro-2H-pyran-2-carboxylic acid

To a solution of (2R,4S,5R,6R)-6-((1R,2R)-1,2-dihydroxy-3-(2-(4-isopropylphenyl)acetamido)propyl)-4-hydroxy-5-(2-hydroxyacetamido)-2-((6-(prop-2-yn-1-yloxy)hexyl)oxy)tetrahydro-2H-pyran-2-carboxylic acid (Cpd. 159, 17.0 mg, 27.3 umol) in anhydrous NMP (176 uL) was added Azido-PEG4-PFP-ester (15.0 mg, 32.8 umol) in anhydrous NMP (176 uL) dropwise, followed by Tetrakis(acetonitrile)copper(l) hexafluorophosphate (15.3 mg, 41.0 umol). The vial was sealed and the mixture was stirred for 45 min at room temperature. The mixture was filtered through a 0.2 μm PTFE filter, a few drops of acetic acid were added, and the filtrate was purified by C18 reverse phase column chromatography using acetonitrile and water (+0.1% TFA) as eluents. The fractions containing the desired product were lyophilized to afford Cpd. 159P as a white solid. Yield: 15.5 mg, 52%; LCMS (ESI) m/z 1080 [M+H]⁺.

### (2R,4S,5R,6R)-6-((1R,2R)-1,2-dihydroxy-3-(2-(4-isopropylphenyl)acetamido)propyl)-2-((6-((1-(18-(2,5-dioxo-2,5-dihydro-1H-pyrrol-1-yl)-15-oxo-3,6,9,12-tetraoxa-16-azaoctadecyl)-1H-1,2,3-triazol-4-yl)methoxy)hexyl)oxy)-4-hydroxy-5-(2-hydroxyacetamido)tetrahydro-2H-pyran-2-carboxylic acid (Cpd. 159M)

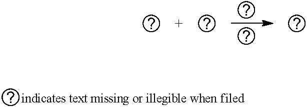

### Synthesis of (2R,4S,5R,6R)-6-((1R,2R)-1,2-dihydroxy-3-(2-(4-isopropylphenyl)acetamido)propyl)-2-((6-((1-(18-(2,5-dioxo-2,5-dihydro-1H-pyrrol-1-yl)-15-oxo-3,6,9,12-tetraoxa-16-azaoctadecyl)-1H-1,2,3-triazol-4-yl)methoxy)hexyl)oxy)-4-hydroxy-5-(2-hydroxyacetamido)tetrahydro-2H-pyran-2-carboxylic acid

To a solution of (2R,4S,5R,6R)-6-((1R,2R)-1,2-dihydroxy-3-(2-(4-isopropylphenyl)acetamido)propyl)-4-hydroxy-5-(2-hydroxyacetamido)-2-((6-((1-(15-oxo-15-(perfluorophenoxy)-3,6,9,12-tetraoxapentadecyl)-1H-1,2,3-triazol-4-yl)methoxy)hexyl)oxy)tetrahydro-2H-pyran-2-carboxylic acid (Cpd. 159P, 15.5 mg, 14.4 umol) in DMF (359 uL) was added n-(2-aminoethyl)maleimide trifluoroacetate salt (3.83 mg, 15.1 umol). The mixture was cooled down to 0° C. and stirred for 10 min. DIEA (7.50 uL, 43.1 umol) was added and the mixture was stirred for 1 h at 0° C. The reaction mixture was filtered and purified by preparatory HPLC using acetonitrile and water (+0.05% TFA) as eluents. The fractions containing the desired product were combined and lyophilized to dryness to afford Cpd. 159M as a white solid. Yield: 8.44 mg, 57%; LCMS (ESI) m/z 1036 [M+H]⁺. ¹H NMR (500 MHz, DMSO-d₆): 8.03 (s, 1H), 7.93 (t, J=6.0 Hz, 1H), 7.91 (t, J=5.5 Hz, 1H), 7.15 (d, J=8.0 Hz, 1H), 7.13 (d, J=8.0 Hz, 2H), 6.99 (s, 2H), 5.50 (s, br, 1H), 4.89 (s, br, 1H), 4.71 (s, br, 1H), 4.50 (t, J=5.5 Hz, 2H), 4.46 (s, 2H), 4.28 (s, br, 1H), 3.87 (m, 2H), 3.82-3.78 (m, 1H), 3.80 (t, J=5.5 Hz, 2H), 3.67-3.60 (m, 2H), 3.56-3.50 (m, 8H), 3.48-3.38 (m, 14H), 3.30-3.26 (m, 2H), 3.21-3.16 (m, 4H), 2.96-2.91 (m, 1H), 2.83 (sept, J=7.0 Hz, 1H), 2.21 (t, J=6. Hz, 2H), 1.54-1.43 (m, 5H), 1.26-1.25 (m, 4H), 1.17 (d, J=7.0 Hz, 6H).

### (2R,4S,5R,6R)-6-((1R,2R)-3-(2-(4-chlorophenyl)acetamido)-1,2-dihydroxypropyl)-4-hydroxy-2-((6-((1-(15-oxo-15-(perfluorophenoxy)-3,6,9,12-tetraoxapentadecyl)-1H-1,2,3-triazol-4-yl)methoxy)hexyl)oxy)-5-ureidotetrahydro-2H-pyran-2-carboxylic acid (Cpd. 86P)

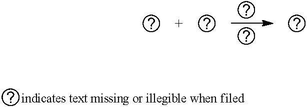

### Synthesis of (2R,4S,5R,6R)-6-((1R,2R)-3-(2-(4-chlorophenyl)acetamido)-1,2-dihydroxypropyl)-4-hydroxy-2-((6-((1-(15-oxo-15-(perfluorophenoxy)-3,6,9,12-tetraoxapentadecyl)-1H-1,2,3-triazol-4-yl)methoxy)hexyl)oxy)-5-ureidotetrahydro-2H-pyran-2-carboxylic acid

To a solution of Azido-PEG4-PFP-ester (13.7 mg, 29.9 umol) in anhydrous NMP (175 uL) was added (2R,4S,5R,6R)-6-((1R,2R)-3-(2-(4-chlorophenyl)acetamido)-1,2-dihydroxypropyl)-4-hydroxy-2-((6-(prop-2-yn-1-yloxy)hexyl)oxy)-5-ureidotetrahydro-2H-pyran-2-carboxylic acid (Cpd. 86, 16.0 mg, 26.7 umol) in anhydrous NMP (175 uL), followed by Tetrakis(acetonitrile)copper(l) hexafluorophosphate (14.9 mg, 40.0 umol). The vial was sealed and the mixture was stirred for 1 hour at room temperature. The mixture was filtered through a 0.2 μm PTFE filter, a few drops of acetic acid were added, and the filtrate was purified by C18 reverse phase column chromatography using acetonitrile and water (+0.1% TFA) as eluents. The fractions containing the desired product were lyophilized to afford Cpd. 86P as a white solid. Yield: 16.9 mg, 60%; LCMS (ESI) m/z 1057 [M+H]⁺.

### (2R,4S,5R,6R)-6-((1R,2R)-3-(2-(4-chlorophenyl)acetamido)-1,2-dihydroxypropyl)-2-((6-((1-(18-(2,5-dioxo-2,5-dihydro-1H-pyrrol-1-yl)-15-oxo-3,6,9,12-tetraoxa-16-azaoctadecyl)-1H-1,2,3-triazol-4-yl)methoxy)hexyl)oxy)-4-hydroxy-5-ureidotetrahydro-2H-pyran-2-carboxylic acid (Cpd. 86M)

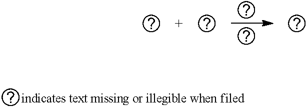

### Synthesis of (2R,4S,5R,6R)-6-((1R,2R)-3-(2-(4-chlorophenyl)acetamido)-1,2-dihydroxypropyl)-2-((6-((1-(18-(2,5-dioxo-2,5-dihydro-1H-pyrrol-1-yl)-15-oxo-3,6,9,12-tetraoxa-16-azaoctadecyl)-1H-1,2,3-triazol-4-yl)methoxy)hexyl)oxy)-4-hydroxy-5-ureidotetrahydro-2H-pyran-2-carboxylic acid

To a solution of (2R,4S,5R,6R)-6-((1R,2R)-3-(2-(4-chlorophenyl)acetamido)-1,2-dihydroxypropyl)-4-hydroxy-2-((6-((1-(15-oxo-15-(perfluorophenoxy)-3,6,9,12-tetraoxapentadecyl)-1H-1,2,3-triazol-4-yl)methoxy)hexyl)oxy)-5-ureidotetrahydro-2H-pyran-2-carboxylic acid (Cpd. 86P, 6.39 mg, 6.04 umol) in DMF (151 uL) was added n-(2-aminoethyl)maleimide trifluoroacetate salt (1.61 mg, 6.35 umol). The mixture was cooled down to 0° C. and stirred for 10 min. DIEA (3.16 uL, 18.1 umol) was added and the mixture was stirred for 1 h at 0° C. The reaction mixture was filtered and purified by preparatory HPLC using acetonitrile and water (+0.05% TFA) as eluents. The fractions containing the desired product were combined and lyophilized to dryness to afford Cpd. 86M as a white solid. Yield: 4.70 mg, 77%; LCMS (ESI) m/z 1013 [M+H]⁺.

### (2R,4S,5R,6R)-6-((1R,2R)-3-(2-(4-chloro-3-fluorophenyl)acetamido)-1,2-dihydroxypropyl)-4-hydroxy-5-(2-hydroxyacetamido)-2-((7-oxo-7-((2-((1-(15-oxo-15-(perfluorophenoxy)-3,6,9,12-tetraoxapentadecyl)-1H-1,2,3-triazol-4-yl)methoxy)ethyl)amino)heptyl)oxy)tetrahydro-2H-pyran-2-carboxylic acid (Cpd. 214P)

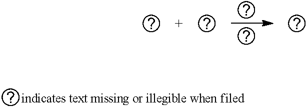

### Synthesis of (2R,4S,5R,6R)-6-((1R,2R)-3-(2-(4-chloro-3-fluorophenyl)acetamido)-1,2-dihydroxypropyl)-4-hydroxy-5-(2-hydroxyacetamido)-2-((7-oxo-7-((2-((1-(15-oxo-15-(perfluorophenoxy)-3,6,9,12-tetraoxapentadecyl)-1H-1, 2, 3-triazol-4-yl)methoxy)ethyl)amino)heptyl)oxy)tetrahydro-2H-pyran-2-carboxylic acid

To a solution of (2R,4S,5R,6R)-6-((1R,2R)-3-(2-(4-chloro-3-fluorophenyl)acetamido)-1,2-dihydroxypropyl)-4-hydroxy-5-(2-hydroxyacetamido)-2-((7-oxo-7-((2-(prop-2-yn-1-yloxy)ethyl)amino)heptyl)oxy)tetrahydro-2H-pyran-2-carboxylic acid (Cpd. 214, 13.5 mg, 19.2 umol) in anhydrous NMP (256 uL) was added Azido-PEG4-PFP-ester (9.67 mg, 21.1 umol), followed by Tetrakis(acetonitrile)copper(l) hexafluorophosphate (17.9 mg, 48.0 umol). The vial was sealed and the mixture was stirred for 1 hour at room temperature. The mixture was filtered through a 0.45 μm PTFE filter. A few drops of acetic acid were added to the filtrate. The filtrate was purified by preparatory HPLC using acetonitrile and water (+0.05% TFA) as eluents. The fractions containing the desired product were lyophilized to afford Cpd. 214P as a white solid. Yield: 9.97 mg, 45%; LCMS (ESI) m/z 1161 [M+H]⁺.

### (2R,4S,5R,6R)-6-((1R,2R)-3-(2-(4-chloro-3-fluorophenyl)acetamido)-1,2-dihydroxypropyl)-2-((7-((2-((1-(18-(2,5-dioxo-2,5-dihydro-1H-pyrrol-1-yl)-15-oxo-3,6,9,12-tetraoxa-16-azaoctadecyl)-1H-1,2,3-triazol-4-yl)methoxy)ethyl)amino)-7-oxoheptyl)oxy)-4-hydroxy-5-(2-hydroxyacetamido)tetrahydro-2H-pyran-2-carboxylic acid (Cpd. 214M)

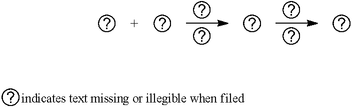

### Synthesis of (2R,4S,5R,6R)-6-((1R,2R)-3-(2-(4-chloro-3-fluorophenyl)acetamido)-1,2-dihydroxypropyl)-2-((7-((2-((1-(18-(2,5-dioxo-2,5-dihydro-1H-pyrrol-1-yl)-15-oxo-3,6,9,12-tetraoxa-16-azaoctadecyl)-1H-1,2,3-triazol-4-yl)methoxy)ethyl)amino)-7-oxoheptyl)oxy)-4-hydroxy-5-(2-hydroxyacetamido)tetrahydro-2H-pyran-2-carboxylic acid

To a solution of (2R,4S,5R,6R)-6-((1R,2R)-3-(2-(4-chloro-3-fluorophenyl)acetamido)-1,2-dihydroxypropyl)-4-hydroxy-5-(2-hydroxyacetamido)-2-((7-oxo-7-((2-((1-(15-oxo-15-(perfluorophenoxy)-3,6,9,12-tetraoxapentadecyl)-1H-1,2,3-triazol-4-yl)methoxy)ethyl)amino)heptyl)oxy)tetrahydro-2H-pyran-2-carboxylic acid (Cpd. 214P, 10.0 mg, 8.61 umol) in DMF (215 uL) was added n-(2-aminoethyl)maleimide trifluoroacetate salt (2.30 mg, 9.04 umol). The mixture was cooled down to 0° C. and stirred for 10 min. DIEA (4.50 uL, 25.8 umol) was added and the mixture was stirred for 1 h at 0° C. The reaction mixture was filtered and purified by preparatory HPLC using acetonitrile and water (+0.05% TFA) as eluents. The fractions containing the desired product were combined and lyophilized to dryness to afford Cpd. 214M as a white solid. Yield: 7.61 mg, 79%; LCMS (ESI) m/z 1117 [M+H]⁺. ¹H NMR (500 MHz, DMSO-d₆): 8.04 (s, 1H), 8.03 (t, J=5.5 Hz, 1H), 7.93 (t, J=6.0 Hz, 1H), 7.85-7.82 (m, 2H), 7.48 (t, J=8.5 Hz, 1H), 7.29 (dd, J=10.5 Hz & 2.0 Hz, 1H), 7.11 (dd, J=8.0 Hz, & 2.0 Hz), 6.99 (s, 2H), 5.49 (s, br, 1H), 4.94 (s, br, 1H), 4.71 (s, br, 1H), 4.50 (t, J=5.5 Hz, 2H), 4.50 (s, 2H), 4.29 (s, br, 1H), 3.90-3.83 (m, 2H), 3.83-3.79 (m, 1H), 3.80 (t, J=5.5 Hz, 2H), 3.67-3.64 (m, 1H), 3.63-3.58 (m, 1H), 3.56-3.51 (m, 8H), 3.48-3.41 (m, 16H), 3.28-3.25 (m, 1H), 3.21-3.17 (m, 6H), 2.21 (t, J=6.5 Hz, 2H), 2.03 (t, J=7.5 Hz, 2H), 1.51 (t, J=12.0 Hz, 1H), 1.48-1.42 (m, 4H), 1.23-1.21 (m, 4H).

### Example 2: Binding Properties of Compounds

### Purpose

To characterize the binding affinities of the synthetic Siglec compounds by surface plasmon resonance (SPR).

### Materials and Methods

The following proteins were used for CD22 ligand binding: biotinylated human Siglec-2/CD22 protein-His-Avitag (Acro Biosystems, Cat #SI2-H82E3), biotinylated mouse Siglec-2-Fc-Avi (in-house production), and recombinant cynomolgus Siglec-2/CD22 His-tag (R&D systems, Cat #9864-SL-050). Prior to immobilization, CD22 proteins were treated with SialoExo (Genovis, Cat #G1-SM1-020) for 4 hours at 37° C. to remove native sialic acid residues.

Capture of biotinylated CD22 constructs was performed using a Cytiva SA chip (BR100398) on a Biacore 8K instrument. Human Siglec 2 was immobilized on channels 1-4 and mouse Siglec 2 on channels 5-8 on the active flow cell (2) and the reference flow cell (1) was immobilized with biotinylated human FGFR2 Fc Avi, an unrelated protein, to account for any nonspecific binding. Human Siglec 2 and Mouse Siglec 2 were diluted in running buffer to 20 ug/mL and injected on the active flow cell (2) for 300 seconds at 5 μL/min, yielding about 3500 RU of captured constructs.

Surface preparation for cyno CD22 protein consisted of amine coupling to Cytiva CM5 chip (Cytiva, Cat #BR100530) using the Cytiva amine coupling kit Type 2 (Cytiva, Cat #BR100633). Following the standard amine coupling protocols, EDC and NHS from the kit were mixed 1:1 and injected over flow cells 1 and 2. Cyno CD22 was then immobilized by injecting 10 ug/mL in Sodium Acetate, pH 5.5 onto flow cell (2) for 420 seconds at 10 uL/min, yielding approximately 2500 RU of captured protein. Any active carboxy groups were then capped with 1M Ethanolamine, pH 8.5 for 420s on flow cells 1 and 2.

Binding experiments of Siglec-ligand conjugated proteins or Siglec ligand compounds were performed on the surfaces prepared above in 10 mM HEPES, 150 mM NaCl, 0.05% Tween-20, pH 7.5, 1% DMSO as the running buffer. Compounds were serially diluted in running buffer from 50 μM to 97.6 nM and injected over both the reference and active flow cells for 90 seconds at 30 μL/min. The compounds were then allowed to dissociate from the surface for 120 seconds. No regeneration was required as the compounds completely dissociated from the surface after 30 seconds. Solvent correction was performed using 0.5, 1, 1.5, and 2% DMSO spike into running buffer without DMSO. Siglec-ligand conjugated proteins were injected in a similar manner from 2 μM to 31.25 nM and the surface was regenerated with 4 M MgCl₂. Data was analyzed using steady-state analysis in Biacore Insight Evaluation Software, version 3.0 for Siglec ligand compounds.

### Results and Conclusions

The biochemical binding properties of the compounds were measured, as shown in the table below. Kd>10 μM was “+”, 1 μM<=Kd<=10 μM was “++”, and Kd<1 μM was +++. Siglec compounds show a range of affinities to human, mouse, and cyno CD22.

| Compound | Affinity mouse | Affinity human | Affinity cyno |
|---|---|---|---|
| Number | CD22 | CD22 | CD22 |
| 1 | +++ | +++ | +++ |
| 2 | ++ | +++ | ++ |
| 3 | ++ | +++ | ++ |
| 4 | +++ | +++ | +++ |
| 5 | ++ | +++ | +++ |
| 6 | ++ | +++ | +++ |
| 7 | + | ++ | + |
| 8 | ++ | ++ | ++ |
| 9 | ++ | ++ | ++ |
| 10 | ++ | ++ | ++ |
| 11 | + | ++ | + |
| 12 | + | ++ | + |
| 13 | + | + | + |
| 14 | ++ | + | + |
| 15 | + | + | + |
| 16 | + | + | + |
| 17 | + | +++ | ++ |
| 18 | ++ | +++ | ++ |
| 19 | ++ | +++ | ++ |
| 20 | ++ | + | + |
| 21 | +++ | +++ | +++ |
| 22 | + | +++ | ++ |
| 23 | + | ++ | ++ |
| 24 | + | ++ | ++ |
| 25 | ++ | + | ++ |
| 26 | +++ | +++ | +++ |
| 27 | + | + | + |
| 28 | + | + | + |
| 29 | + | ++ | + |
| 30 | + | ++ | ++ |
| 31 | + | ++ | ++ |
| 32 | + | + | + |
| 33 | + | +++ | ++ |
| 34 | ++ | +++ | ++ |
| 35 | ++ | +++ | ++ |
| 36 | ++ | +++ | ++ |
| 37 | +++ | +++ | +++ |
| 38 | +++ | +++ | +++ |
| 39 | ++ | +++ | +++ |
| 40 | ++ | +++ | +++ |
| 41 | ++ | ++ | ++ |
| 42 | ++ | +++ | +++ |
| 43 | +++ | +++ | +++ |
| 44 | ++ | +++ | ++ |
| 45 | ++ | +++ | ++ |
| 46 | ++ | +++ | ++ |
| 47 | +++ | +++ | +++ |
| 48 | ++ | +++ | ++ |
| 49 | +++ | +++ | +++ |
| 50 | +++ | +++ | ++ |
| 51 | +++ | +++ | ++ |
| 52 | +++ | +++ | ++ |
| 53 | ++ | +++ | ++ |
| 54 | + | +++ | ++ |
| 55 | +++ | ++ | ++ |
| 56 | +++ | +++ | ++ |
| 57 | + | ++ | ++ |
| 58 | ++ | ++ | ++ |
| 59 | ++ | +++ | ++ |
| 60 | ++ | ++ | ++ |
| 61 | +++ | +++ | +++ |
| 62 | ++ | ++ | ++ |
| 63 | +++ | +++ | +++ |
| 64 | +++ | +++ | +++ |
| 65 | + | + | + |
| 66 | + | ++ | + |
| 67 | ++ | ++ | ++ |
| 68 | ++ | +++ | ++ |
| 69 | + | ++ | + |
| 70 | ++ | +++ | ++ |
| 71 | + | ++ | ++ |
| 72 | + | ++ | + |
| 73 | +++ | +++ | +++ |
| 74 | ++ | ++ | ++ |
| 75 | + | + | + |
| 76 | ++ | +++ | ++ |
| 77 | +++ | +++ | +++ |
| 78 | ++ | +++ | ++ |
| 79 | + | ++ | + |
| 80 | ++ | ++ | ++ |
| 81 | + | ++ | ++ |
| 82 | + | ++ | + |
| 83 | ++ | +++ | ++ |
| 84 | ++ | +++ | ++ |
| 85 | ++ | +++ | +++ |
| 86 | +++ | +++ | ++ |
| 87 | ++ | +++ | ++ |
| 88 | + | ++ | ++ |
| 89 | ++ | ++ | ++ |
| 90 | ++ | +++ | +++ |
| 91 | + | ++ | +++ |
| 92 | + | +++ | +++ |
| 93 | + | ++ | + |
| 94 | ++ | ++ | ++ |
| 95 | ++ | +++ | +++ |
| 96 | +++ | +++ | +++ |
| 97 | +++ | +++ | +++ |
| 98 | ++ | +++ | +++ |
| 99 | +++ | +++ | +++ |
| 100 | +++ | +++ | +++ |
| 101 | ++ | ++ | ++ |
| 102 | ++ | ++ | ++ |
| 103 | ++ | +++ | +++ |
| 104 | + | + | + |
| 105 | +++ | +++ | +++ |
| 106 | ++ | +++ | +++ |
| 107 | ++ | +++ | +++ |
| 108 | +++ | +++ | +++ |
| 109 | +++ | +++ | +++ |
| 110 | +++ | +++ | +++ |
| 111 | ++ | ++ | +++ |
| 112 | +++ | +++ | +++ |
| 113 | ++ | +++ | +++ |
| 114 | +++ | +++ | +++ |
| 115 | ++ | +++ | +++ |
| 116 | ++ | +++ | +++ |
| 117 | +++ | +++ | +++ |
| 118 | + | ++ | ++ |
| 119 | ++ | ++ | ++ |
| 120 | ++ | +++ | +++ |
| 121 | ++ | +++ | +++ |
| 122 | +++ | +++ | +++ |
| 123 | ++ | +++ | +++ |
| 124 | +++ | +++ | ++ |
| 125 | ++ | +++ | ++ |
| 126 | ++ | ++ | ++ |
| 127 | ++ | +++ | +++ |
| 128 | +++ | +++ | +++ |
| 129 | ++ | +++ | +++ |
| 130 | ++ | +++ | +++ |
| 131 | +++ | +++ | +++ |
| 132 | + | ++ | +++ |
| 133 | + | +++ | +++ |
| 134 | ++ | +++ | +++ |
| 135 | +++ | +++ | +++ |
| 136 | ++ | +++ | +++ |
| 137 | + | + | ++ |
| 138 | ++ | ++ | +++ |
| 139 | +++ | ++ | ++ |
| 140 | ++ | ++ | ++ |
| 141 | +++ | +++ | ++ |
| 142 | +++ | +++ | +++ |
| 143 | ++ | +++ | +++ |
| 144 | ++ | ++ | +++ |
| 145 | ++ | ++ | ++ |
| 146 | ++ | ++ | +++ |
| 147 | ++ | ++ | ++ |
| 148 | +++ | +++ | ++ |
| 149 | ++ | +++ | ++ |
| 150 | ++ | +++ | ++ |
| 151 | ++ | ++ | + |
| 152 | +++ | ++ | ++ |
| 153 | +++ | ++ | ++ |
| 154 | +++ | +++ | +++ |
| 155 | ++ | +++ | +++ |
| 156 | ++ | +++ | +++ |
| 157 | +++ | +++ | +++ |
| 158 | ++ | +++ | ++ |
| 159 | +++ | +++ | +++ |
| 160 | ++ | +++ | +++ |
| 161 | +++ | +++ | +++ |
| 162 | ++ | ++ | +++ |
| 163 | ++ | ++ | ++ |
| 164 | ++ | ++ | ++ |
| 165 | ++ | +++ | ++ |
| 166 | ++ | +++ | ++ |
| 167 | ++ | +++ | ++ |
| 168 | ++ | ++ | ++ |
| 169 | + | ++ | ++ |
| 170 | ++ | ++ | ++ |
| 171 | ++ | ++ | ++ |
| 172 | ++ | ++ | ++ |
| 173 | + | ++ | ++ |
| 174 | ++ | ++ | ++ |
| 175 | ++ | ++ | ++ |
| 176 | +++ | +++ | +++ |
| 177 | + | ++ | ++ |
| 178 | + | ++ | ++ |
| 179 | + | +++ | ++ |
| 180 | ++ | + | ++ |
| 181 | ++ | ++ | ++ |
| 182 | ++ | ++ | ++ |
| 183 | ++ | ++ | +++ |
| 184 | ++ | ++ | ++ |
| 185 | ++ | ++ | +++ |
| 186 | +++ | +++ | +++ |
| 187 | +++ | +++ | +++ |
| 188 | ++ | +++ | +++ |
| 189 | ++ | ++ | +++ |
| 190 | ++ | +++ | +++ |
| 191 | + | +++ | +++ |
| 192 | + | ++ | ++ |
| 193 | ++ | ++ | ++ |
| 194 | ++ | ++ | +++ |
| 195 | +++ | +++ | +++ |
| 196 | ++ | +++ | +++ |
| 197 | + | + | + |
| 198 | + | + | + |
| 199 | + | + | + |
| 200 | + | + | + |
| 201 | + | + | + |
| 202 | ++ | + | +++ |
| 203 | ++ | +++ | +++ |
| 204 | +++ | +++ | ++ |
| 205 | +++ | +++ | +++ |
| 206 | ++ | +++ | +++ |
| 207 | ++ | ++ | ++ |
| 208 | + | ++ | +++ |
| 209 | ++ | ++ | ++ |
| 210 | +++ | ++ | +++ |
| 211 | +++ | +++ | +++ |
| 212 | +++ | +++ | +++ |
| 213 | +++ | +++ | +++ |
| 214 | +++ | +++ | +++ |
| 215 | + | +++ | ++ |
| 216 | +++ | +++ | +++ |
| 217 | ++ | +++ | +++ |
| 218 | +++ | +++ | +++ |
| 219 | +++ | +++ | +++ |
| 220 | +++ | +++ | +++ |
| 221 | +++ | +++ | +++ |
| 222 | +++ | +++ | +++ |
| 223 | ++ | +++ | ++ |
| 224 | +++ | +++ | +++ |
| 225 | + | + | + |
| 226 | + | + | + |
| 227 | ++ | +++ | +++ |
| 228 | ++ | ++ | +++ |
| 229 | ++ | ++ | ++ |
| 230 | ++ | ++ | ++ |
| 231 | ++ | +++ | +++ |
| 232 | + | ++ | ++ |
| 233 | ++ | ++ | ++ |
| 234 | ++ | ++ | ++ |
| 235 | +++ | +++ | +++ |
| 236 | ++ | ++ | ++ |
| 237 | ++ | ++ | ++ |
| 238 | ++ | ++ | ++ |
| 239 | ++ | ++ | ++ |
| 240 | +++ | +++ | +++ |
| 241 | ++ | +++ | +++ |
| 242 | ++ | ++ | ++ |
| 243 | ++ | ++ | ++ |
| 244 | +++ | +++ | +++ |
| 245 | +++ | +++ | +++ |
| 246 | +++ | +++ | +++ |
| 247 | ++ | ++ | ++ |
| 248 | ++ | +++ | ++ |
| 249 | ++ | ++ | ++ |
| 250 | ++ | +++ | ++ |
| 251 | ++ | ++ | +++ |
| 252 | ++ | +++ | +++ |
| 253 | +++ | +++ | +++ |
| 254 | ++ | +++ | +++ |
| 255 | ++ | +++ | +++ |
| 256 | ++ | +++ | +++ |
| 257 | ++ | +++ | +++ |
| 258 | +++ | +++ | +++ |
| 259 | +++ | +++ | +++ |
| 260 | ++ | ++ | ++ |
| 261 | +++ | +++ | +++ |
| 262 | ++ | +++ | +++ |
| 263 | ++ | +++ | +++ |
| 264 | ++ | +++ | +++ |
| 265 | +++ | +++ | +++ |
| 266 | ++ | +++ | +++ |
| 267 | ++ | +++ | +++ |
| 268 | + | ++ | ++ |
| 269 | + | ++ | + |
| 270 | + | ++ | ++ |
| 271 | + | ++ | ++ |
| 272 | + | ++ | ++ |
| 273 | + | ++ | ++ |
| 274 | + | + | + |
| 275 | ++ | +++ | +++ |

### Example 3: Analysis of Synthetic CD22 Binding Specificity Across Various Siglec Receptor Family Members

### Purpose

The purpose of this study was to examine Siglec ligand specificity for Siglec-2 in comparison to other Siglec family members. A competitive bead-based flow cytometry assay was developed to assess selectivity of CD22 ligands for Siglec proteins. Briefly, the assay employs a streptavidin bead coated with biotinylated CD22 ligand. The beads are then incubated with human Siglec-2-AF647 at a low concentration and competitive unlabeled Siglec proteins were added. A decrease in Siglec-2-AF647 indicates competitive binding of the CD22 ligand beads with the unlabeled Siglec protein.

### Materials and Methods

The following recombinant Siglec proteins were obtained from commercial sources: human Siglec-2-Fc fusion (R&D systems, Cat. No. 1968-SL-050), human Siglec-3-Fc fusion (Acro Biosystems, Cat. No. CD3-H5257), human Siglec-4a-Fc fusion (Acro Biosystems, Cat. No. MAG-H5254), human Siglec-15-Fc fusion (Acro Biosystems, Cat. No. SG5-H5253), Mouse Siglec-1-Fc fusion (R&D systems, Cat. No. 5610-SL-50), and human Siglec-2-Fc fusion labeled with AF647 (R&D systems, Cat. No. AFR1968-020)

Dynabeads M−280 Streptavidin-coated beads (20 μl of 6.7×10⁸ beads per mL, Thermo Scientific, Cat. No. 11205D) were washed with (−/−) phosphate buffered saline containing 1% bovine serum albumin and 0.1% sodium azide (FACS buffer), centrifuged beads at 350 g for 1 min and aspirated the supernatant. Beads were resuspended in 200 μl of FACS buffer and then incubated with biotinylated-CD22 ligand (5 μM) overnight at 4° C. Separate beads were coated for each CD22 ligand tested.

In a 96-well U-bottom plate, human Siglec-2 Fc AF647 at a fixed concentration of 10 nM was added to an increasing titration (0-200 nM) of non-fluorescently labeled human Siglec-2 Fc protein (no AF647) in FACS buffer. Additional conserved human Siglec proteins were tested at the following concentrations: mouse Siglec-1 Fc at 150 nM; human Siglec-3-Fc at 200 nM; human Siglec-4-Fc at 200 nM; human Siglec-15-Fc at 200 nM. CD22 ligand-beads were washed and resuspended in FACS buffer.

Approximately 33,500 CD22 ligand-beads were added per well and mixed by pipette. After 60 min at room temperature, protected from light, samples were analyzed by flow cytometry at 150 events per sec. Flow cytometry was performed on a Biorad ZE5 analyzer and data was processed using FlowJo LLC. The single bead populations were gated out to obtain the median fluorescence intensity (MFI) of AF647, which indicates binding of the human Siglec-2 Fc-AF647 to the surface of the CD22 ligand-conjugated bead. MFI values were used to generate binding curves (variable slope four parameters) in GraphPad Prism.

### Results and Conclusions

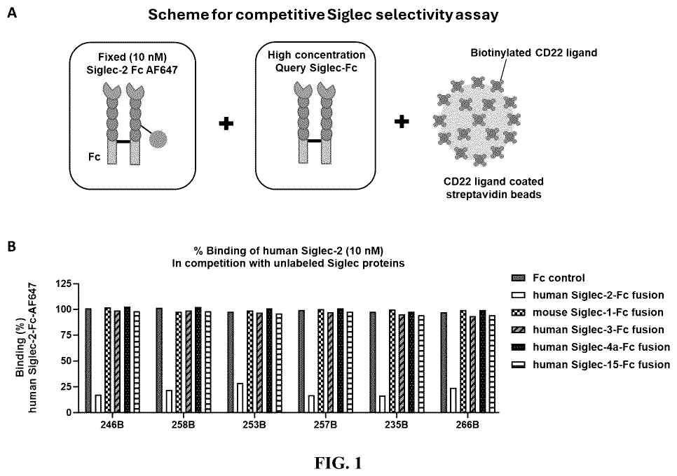

FIG. 1A shows a scheme for the competitive Siglec assay where a fixed concentration of human Siglec-2-Fc-AF647 is incubated with CD22 ligand-bead and a competitor Siglec-Fc fusion protein. FIG. 1B shows the percent binding of the AF647-labeled human Siglec-2 high concentrations of competitive Siglec protein (mouse Siglec-1 Fc at 150 nM; human Siglec-3-Fc at 200 nM; human Siglec-4-Fc at 200 nM; human Siglec-15-Fc at 200 nM) to the various CD22 ligand-beads. Each of the CD22 ligands examined demonstrated a decrease in binding of the human Siglec-2-Fc-AF647 where a high concentration of unlabeled human Siglec-2-Fc fusion was present, indicating that CD22-ligands bind human Siglec-2-Fc fusion.

Human Siglec 3,4a, 15, and mouse Siglec-1 was also tested for competitive displacement of AF647-label Siglec-2. Those Siglecs showed no significant levels of AF647-labeled Siglec-2 signal decreases, indicating that they do not bind to the CD22 ligands. An isotype control antibody was also used as a competitor protein and confirmed that binding was specific for Siglec and not Fc fusion (data not shown). Overall, this panel of CD22 ligands shows high specificity for Siglec-2 and does not show binding to other Siglec proteins tested.

### Example 4: Antibody (Anti-Mouse IgD Human IgG1 Chimeric Antibody, Anti-Human IgM Human IgG1 Chimeric Antibody, and Adalimumab) Expression, Purification, and Analytics

### Materials and Methods

For antibody expression, the ExpiFectamine 293 Transfection kit (Life Technologies, A14524) was used to transfect suspension Expi293F cells (Life Technologies, A14527) with Heavy Chain and Light Chain plasmids (pTT5-based) at a 1:1 ratio. Media was harvested 3-6 days post-transfection by centrifugation and filtered using 0.2 μm PES vacuum sterile single-use filter unit (ThermoScientific, Cat. No. 5670020)

Purification was performed with 1.5 mL MabSelect Sure resin (Cytiva/GE Cat #: 17-5438-03) for each 250 mL culture supernatant. Briefly, each column was equilibrated with PBS pH 7.2 and loaded with culture supernatant. After the loading step, the column was washed with PBS pH 7.2 and eluted with 10 mL IgG Elution buffer (Thermo Scientific, Cat. No. 21004). The pH of the elution pool was adjusted with 1 mL 1 M Sodium Phosphate pH 6.5 for each 10 mL elution pool. Finally, buffer exchange was performed with PBS pH 7.2 using a 30 kDa Amicon Ultra-15 Centrifugal Filter Unit.

Analysis of endotoxin content was performed using the Charles River Endosafe PTS 0.01-1 EU/ml detection. Size exclusion chromatography was performed on an Agilent Chemstation HPLC-SEC with a Sepax-Zenix SEC-300, 200 mm×7.8 mm ID, 3 uM column. Capillary gel electrophoresis (cGE) was performed on a Caliper LabChip GXII Protein 200 with the Perkin Elmer Chip (Cat. No. 760499). LC-MS analysis was performed on SciEX LC 5600+, ExionLC AD, Analyst TF 1.8.1 with an Agilent AdvanceBio Desalting-RP, Column 1000A, 10 μm.

### Protein-Sigtlec Ligand Conjugation

Pentafluorophenyl (PFP) conjugatable Siglec Ligand linker was added to reaction mixtures at a molar ratio of 4-30 times above protein based on desired degree of labeling in the presence of 10% v/v of 50 mM Sodium Tetraborate pH 8.5 and 10% v/v DMSO. Reactions were incubated for 3 hours at 25° C. After the 3 h incubation period, 10% v/v of 1 M Tris-HCl pH 8.0 was added to quench the unreacted linker-payload. Neutralized reactions were then allowed to incubate at 25° C. for 15 min.

### Protein-Sigtlec Ligand Conjugation to Site-Specific Cysteines

Two different mutation sites were employed in the heavy chain region of the antibodies to incorporate new cysteine sites, L443C and K290C. The cysteine-engineered protein was diluted in a solution containing 1 mM diethylenetriaminepentaacetic acid (DTPA) and 100 mM HEPES at pH 7.0. To this mixture, tris(2-carboxyethyl)phosphine (TCEP) was added in a 20-fold molar excess above protein. The resulting mixture was incubated at 37° C. for 2 h. After incubation, excess TCEP was removed using a desalting column equilibrated with PBS at pH 7.2.

The reduced protein was then re-oxidized in a solution containing 100 mM HEPES at pH 7.0, 1 mM DTPA, and 2 mM dehydroascorbic acid (DHA), and incubated at 4° C. for 16 h. Maleimide-conjugatable Siglec Ligand linkers in 10% v/v dimethylacetamide (DMA) were added to the reaction mixtures at a molar ratio of 10-60 times above protein, based on the desired degree of labeling. Reactions were incubated at 25° C. for 2 h. After the incubation period, 10% v/v of 100 mM reduced glutathione at pH 7.0 was added to quench any unreacted maleimide linkers. The neutralized reactions were incubated at 25° C. for an additional 15 min.

### Protein Sigtlec-Ligand Conjugate Purification

Quenched conjugation reactions are purified by preparative size exclusion chromatography at 4° C. using either Superdex 200 Increase 10/300 GL or HiLoad 16/600 Superdex 200 pg at a flow rate of 0.75 mL/min, with PBS pH 7.2.

### Analysis of Protein-Sigtlec Ligand Conjugates

The “LDR”, or Ligand-to-Drug ratio, was measured for each conjugate preparation by LC/MS, by evaluating the relative abundances of species varying in the degree of conjugation, as described here. Random conjugation methods (to lysine/amines in this case) result in a mixture of species varying in the degree of conjugation per adalimumab species. Such a series of molecular species can be represented as:

$${\sum }_{i = 1}^{m} {[ {X}_{n} L ]}_{i} Y$$

In this statement, each biotherapeutic, Y (adalimumab, in this case), is covalently bound to a Siglec Ligand as defined by XₙL, where X is a sialic acid species of valency, n, with a Siglec Ligand-to-biotherapeutic ratio that varies between 1 and m. All species have the same Sialic Acid valency, n (monovalent, bivalent, or trivalent)

As a total measure of the degree of conjugation in such an ensemble of species with varying degrees of conjugation, the LDR can be defined as follows: LDR is a weighted average of the individual Siglec ligand-to-biotherapeutic ratios (integer value m) in a mixture of species varying in said ratio, and Pᵢ(0≤Pᵢ≤1, with Σᵢ₌₁ⁿPᵢ=1) representing the fractional abundance of each species in the mixture:

$$LDR = {\sum }_{i = 1}^{n} {P}_{i} {m}_{i}$$

Protein-siglec ligand conjugates were analyzed using the following parameters.

### LC-MS

Mass spectrometer: SciEX LC 5600+, ExionLC AD, Analyst TF 1.8.1
HPLC: Agilent AdvanceBio Desalting-RP, Column 1000A, 10 um
2.1 mm×12.5 mm, flow rate 400 ul/min, Sample load: 5 ug
Buffer A: Water+0.1% Formic Acid
Buffer B: Acetonitrile+0.1% Formic Acid

### Analytical SEC

Superose 6 Increase 10/300 GL
Injection Volume: 25 uL at 1 mg/mL of sample.
Buffer: 50 mM Sodium Phosphate+400 mM Sodium Perchlorate, pH 6.2
Flow rate: 0.7 mL/min
Run time 40 min at 25° C.

### Capillary Gel Electrophoresis

Caliper LabChip GXII Protein 200

### Endotoxin Measurement

Charles River Endosafe PTS Cartridge 0.01-1 EU/mi
Lonza Pyrogene C Endpoint Endotoxin Assay

### Results and Conclusions

The results below show a summary of conjugates that were described herein.

| | Conjugation | | | Compound | Conjugate |
|---|---|---|---|---|---|
| Protein | site | LDR | Conjugation type | ID | ID |
| Anti-mouse-IgD | | 6 | Pentafluorophenyl | 41P | NHC041 |
| | | | coupling to lysine | | |
| Anti-mouse-IgD | | 6 | Pentafluorophenyl | 53P | NHC053 |
| | | | coupling to lysine | | |
| Anti-mouse-IgD | | 7 | Pentafluorophenyl | 111P | NHC111 |
| | | | coupling to lysine | | |
| Anti-mouse-IgD | | 7 | Pentafluorophenyl | 110P | NHC110 |
| | | | coupling to lysine | | |
| Anti-mouse-IgD | | 6 | Pentafluorophenyl | 112P | NHC112 |
| | | | coupling to lysine | | |
| Anti-mouse-IgD | | 6 | Pentafluorophenyl | 86P | NHC086 |
| | | | coupling to lysine | | |
| Anti-mouse-IgD | | 6 | Pentafluorophenyl | 103P | NHC103 |
| | | | coupling to lysine | | |
| Anti-human-IgM | HC-K290C | 2 | Maleimide coupling to | 41M | NHC008 |
| | | | cysteine | | |
| Anti-human-IgM | HC-K290C | 2 | Maleimide coupling to | 110M | NHC009 |
| | | | cysteine | | |
| Anti-human-IgM | HC-K290C | 2 | Maleimide coupling to | 86M | NHC010 |
| | | | cysteine | | |
| Anti-human-IgM | HC-K290C | 2 | Maleimide coupling to | 246M | NHC011 |
| | | | cysteine | | |
| Anti-human-IgM | HC-K290C | 2 | Maleimide coupling to | 161M | NHC012 |
| | | | cysteine | | |
| Anti-human-IgM | HC-K290C | 2 | Maleimide coupling to | 159M | NHC013 |
| | | | cysteine | | |
| Anti-human-IgM | HC-K290C | 2 | Maleimide coupling to | 100M | NHC014 |
| | | | cysteine | | |
| Anti-human-IgM | HC-K290C | 2 | Maleimide coupling to | 227M | NHC015 |
| | | | cysteine | | |
| Anti-human-IgM | HC-K290C | 2 | Maleimide coupling to | 214M | NHC016 |
| | | | cysteine | | |
| Anti-human-IgM | HC-K290C | 2 | Maleimide coupling to | 258M | NHC017 |
| | | | cysteine | | |
| Anti-human-IgM | HC-K290C | 2 | Maleimide coupling to | 252M | NHC019 |
| | | | cysteine | | |
| Anti-human-IgM | HC-K290C | 2 | Maleimide coupling to | 247M | NHC020 |
| | | | cysteine | | |
| Adalimumab | | 11 | Pentafluorophenyl | 157P | NHC021 |
| | | | coupling to lysine | | |
| Adalimumab | | 10 | Pentafluorophenyl | 110P | NHC022 |
| | | | coupling to lysine | | |
| Adalimumab | | 10 | Pentafluorophenyl | 41P | NHC023 |
| | | | coupling to lysine | | |
| Adalimumab | HC-L443C | 2 | Maleimide coupling to | 41M | NHC024 |
| | | | cysteine | | |
| Adalimumab | HC-L443C | 2 | Maleimide coupling to | 157M | NHC025 |
| | | | cysteine | | |
| Adalimumab | HC-L443C | 2 | Maleimide coupling to | 110M | NHC026 |
| | | | cysteine | | |
| Adalimumab | HC-L443C | 2 | Maleimide coupling to | 112M | NHC027 |
| | | | cysteine | | |
| Adalimumab | HC-L443C | 2 | Maleimide coupling to | 176M | NHC028 |
| | | | cysteine | | |
| Adalimumab | HC-L443C | 2 | Maleimide coupling to | 221M | NHC029 |
| | | | cysteine | | |

All conjugates were purified to homogeneity for oligomeric species, with the intended oligomeric structure (e.g., monomer, dimer, trimer) being purified by preparative size exclusion chromatography. All control protein preparations and conjugate preparations were >99% pure by analytical SEC.

### Example 5: Suppression of B Cell Receptor-Mediated Activation of Mouse Primary B Cells by Siglec Ligand-Anti-IgD Antibody Conjugates

### Purpose and Introduction

The purpose of this experiment was to test for suppressive effects on mouse B cell activation of B cell receptor (BCR) agonist IgG-Siglec Ligand conjugates.

The platform technology invention described herein rests on the premise that activation of B cells through their clonotypic B cell receptor can be suppressed through physical recruitment of the CD22/Siglec-2 inhibitory coreceptor to co-engaged B cell receptor. CD22 recruited to the B cell receptor is phosphorylated on its ITIM cytoplasmic motif tyrosines by virtue of its proximity to the high local protein kinase activity at the B cell receptor. Phosphorylated CD22 then recruits phosphatases, such as SHP-1 and SHP-2, to the cell surface, in proximity of the B cell activation complex. Such elevated local phosphatase activity dephosphorylates components of the B cell activation complex necessary for B cell activation, thus shutting down responses to B cell receptor engagement. Under normal circumstances, the Siglec-2 immunoinhibitory mechanism acts as a check on aberrant B cell activation, safeguarding against autoreactive antibody production, hyperinflammation, and autoimmunity. The described platform technology invention exploits this natural phenomenon to cloak foreign proteins as self, dampening B cell activation only on naïve B cell clones that are specific for the given foreign protein and thus blocking immunoglobulin production against the foreign protein, while leaving B cell responses to other antigens intact.

The high diversity of primary B cell populations, and high diversity of B cell receptor sequences and clones (as high as 10¹² per human), presents a challenge for studying BCR agonism in vitro with a single, well-defined BCR antigen. For this reason, pan-BCR activators, such as anti-IgD or anti-IgM antibodies, that can bind, crosslink, and activate the BCR—regardless of B cell/BCR clonality—are used to evaluate BCR activation in vitro. In the experiments described in this example, an anti-mouse IgD monoclonal antibody is used either in parental IgG form or as IgG-Siglec-Ligand conjugates to study the effects of Siglec-2-B cell receptor co-engagement on B cell activation.

To control for impacts of anti-IgD conjugation on BCR binding potency, competition binding assays were used to assess binding activity of Siglec Ligand conjugates and ensure that apparent suppressive effects were due to Siglec-2×BCR co-engagement and not a general damaging of anti-IgD for receptor binding.

### Materials & Methods—In Vitro Murine B-Cell Activation and Surface IgD Competition Assays

Anti-IgD and Anti-IgD-Siglec Ligand test articles were prepared as described in Example 4.

Splenocytes from C57BL/6 mice were harvested into single cell suspension, subjected to red cell lysis using ACK buffer, and plated at a concentration of 200,000 cells per well in round bottom 96 well plates in complete RPMI media. Cells were stimulated by the addition of increasing concentration of anti-mouse IgD or Siglec ligand-conjugated anti-mouse IgD and incubated for 3 hours at 37° C., 5% CO₂. Cell activation was assessed by flow cytometry.

To measure B cell activation following the described stimulation, cells were washed twice by centrifugation at 350 g for 5 minutes and adding staining buffer (1% bovine serum albumin/0.1% sodium azide/1×phosphate buffered saline). Cells were then resuspended in staining buffer and incubated with Fc-block (Biolegend) for five minutes before the addition of anti-CD45, anti-CD19, anti-CD69, and anti-CD86 antibodies (BD Biosciences, Biolegend, Fisher). Cells were then incubated in the dark for 30 minutes at room temperature. Cells were then washed three times with staining buffer and analyzed on ZE5 (BioRad). Data analysis was performed using FlowJo (v10.8.0) software.

### General Gating Strategy:

1. Lymphocyte gate set on FSC 488 10-A/SSC 488-10-A
2. Single cells gate set on FSC 488 10-H/FSC 488-10-A
3. CD45+ gate set on CD45-FITC+ population using CD45-FITC-A/SSC 488-10-A
4. % CD69+ gate set on CD69-Alexa647-A/CD19-BV421-A
5. Mean Fluorescence Intensity (MFI) gate is set on CD19+ cells using CD69-Alexa647-A/CD19-BV421-A

Competition binding analysis of Siglec ligand-conjugated anti-mouse IgD and anti-mouse IgD binding was performed on splenocytes that were harvested as described above. Cells were prepared at a concentration of 4×10⁶ cells/mL in staining buffer and then incubated with mouse Fc-block (BD Biosciences) for five minutes. Following this incubation, anti-CD19 and anti-CD45 antibodies were added. Cells were plated at 50 μL per well (200,000 cells/well) in round bottom 96 well plates in staining buffer. Anti-mouse-IgD-AlexaFluor647 at a fixed concentration of 2.88 nM and an increasing titration (0-40 nM) of non-fluorescently labeled Siglec ligand-conjugated anti-mouse IgD was added to the cells simultaneously. Cells were placed on ice for 30 minutes in the dark. Afterwards, cells were washed twice by centrifugation with staining buffer and antibody binding was analyzed using flow cytometry (ZE5, BioRad) and measuring the AlexaFluor647 mean fluorescence intensity (MFI) of at least 10,000 cells.

### Results and Conclusions

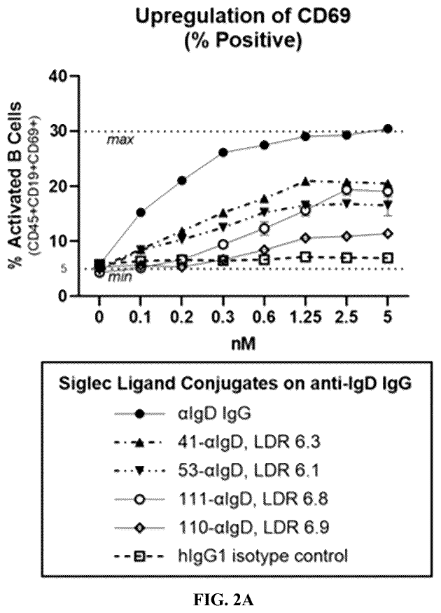

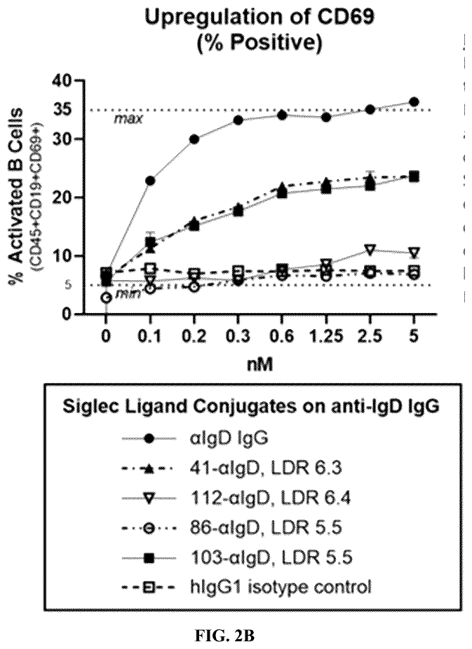

FIGS. 2A and 2B depict an in vitro primary mouse B cell activation assay testing for the importance of CD22 engagement for Siglec Ligand-conjugate-mediated suppression of B cell receptor activation. Mouse primary B cells were treated with either a B cell receptor agonizing anti-IgD antibody or various anti-IgD-Siglec Ligand conjugates. In this case, conjugates (NHC041, NHC053, NHC086, NHC103, NHC111, and NHC112) carried derivatives of molecules 41, 53, 86, 103, 110, 111 or 112 covalently coupled to anti-IgD antibody using lysine chemistry as described in Example 4. The anti-IgD antibodies were conjugated at an LDR of 10 and a competition binding assay was used to confirm that anti-IgD binding was maintained. No significant changes in binding affinity were observed with the anti-IgD conjugates with the high degree of labeling (data not shown), indicating that any suppression observed was not due to inhibition of IgD binding.

Where parental anti-IgD antibody induces a strong, concentration-dependent increase in % CD69-positive cells and in CD69 MFI, Siglec Ligand-anti-IgD conjugates show varying degrees of suppression. Moreover, the degree of ADA suppression correlates well with the affinity of the CD22-ligand that is conjugated to the anti-IgM. Overall, these results supported that higher affinity Siglec Ligands generally increased suppression of BCR activation through Siglec-2/CD22 co-engagement activity.

### Example 6: Suppression of B Cell Receptor-Mediated Activation of Human Primary B Cells by Siglec Ligand-Anti-IgM Antibody Conjugates

### Purpose

The purpose of this experiment was to test for suppressive effects on human B cell activation with B cell receptor agonist IgG-Siglec Ligand conjugates. This experiment is analogous to the one described in Example 5, with the focus here on primary human, PBMC-derived B cells, rather than the primary mouse splenocytes. In addition to the human comparison, this experiment sought to further differentiate the ADA suppression capacity of CD22 ligands by using a lower LDR anti-IgM antibody.

### Materials and Methods

Anti-IgM and Anti-IgM-Siglec Ligand test articles were prepared as described in Example 4. In this case, the anti-IgM constructs were site-specifically conjugated at the heavy chain site K290C to an LDR of 2.

Human PBMCs (StemExpress) were plated at a concentration of 200,000 cells per well in round bottom 96 well plates in complete RPMI media. Cells were stimulated for 18 hours by the addition of increasing concentration of anti-human IgM or Siglec ligand-conjugated anti-human IgM. B cell activation was assessed by flow cytometry.

To measure B cell activation following the described stimulation, cells were washed twice by spinning cells at 1200 rpm for 5 minutes and rinsing with PBS. Cells were resuspended in staining buffer (1% bovine serum albumin/0.1% sodium azide/1×phosphate buffered saline) and incubated with Fc-block (BD Biosciences) for five minutes before the addition of anti-CD45, anti-CD19, anti-CD69, and anti-CD86 antibodies (BD Biosciences, Biolegend, Fisher). Cells were incubated in the dark for an additional 30 minutes at room temperature, then washed three times with staining buffer and then analyzed on ZE5 (BioRad). Data analysis was performed using FlowJo (v10.8.0) software.

Competition of Siglec ligand-conjugated anti-human IgM binding was carried out on human PBMCs from the same donors used in assays described above. For this assay, cells were seeded at a concentration of 200,000 cells per well in round bottom 96 well plates in complete RPMI media. Cells were subsequently incubated with human Fc-block (BD Biosciences) for five minutes. Following this incubation period, 2.4 nM anti-human-IgM-AlexaFluor647 was added to the cells along with RPMI alone or an increasing titration of non-fluorescently labeled Siglec ligand-conjugated anti-human IgM and anti-CD19. Cells were incubated at 4° C. for 30 minutes in the dark. After incubation, cells were washed twice by centrifugation with staining buffer (1% bovine serum albumin/0.1% sodium azide/1×phosphate buffered saline) and antibody binding was analyzed by flow cytometry (ZE5, BioRad) by determination of the mean fluorescence intensity (MFI) of at least 5,000 cells.

### Results and Conclusions

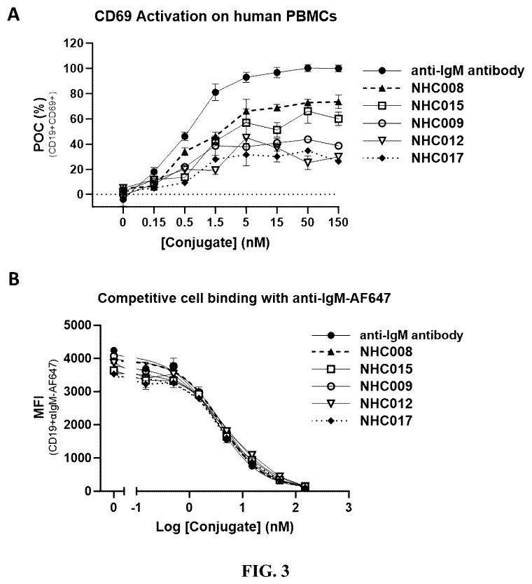

FIG. 3A shows representative B cell stimulatory activities of anti-IgM and anti-IgM-CD22 Ligand conjugates in dose titration experiments with an activation readout of CD69 upregulation. CD69 levels on the different treatment groups are evaluated through the percentage of cells that are CD69-positive. The table below shows a summary of all the compounds tested in the assay with the percentage of cells that are CD69-positive at 150 nM.

| Molecule | % CD69 at 150 nM |
|---|---|
| Anti-human-IgM | 98.9 |
| NHC008 | 73.6 |
| NHC009 | 38.8 |
| NHC010 | 43.4 |
| NHC011 | 57.8 |
| NHC012 | 30.1 |
| NHC013 | 32.2 |
| NHC014 | 35.6 |
| NHC015 | 60.1 |
| NHC016 | 28.8 |
| NHC017 | 26.3 |
| NHC018 | 36.1 |
| NHC019 | 68.7 |

Where parental anti-IgM IgG induces a strong, concentration-dependent increase in % CD69-positive cells and in CD69 MFI, Siglec Ligand-anti-IgD conjugates show very strongly suppressed activation. The degree of suppression increases with affinity of CD22 ligand. Moreover, as shown in FIG. 3B, there is no interference of the conjugates with IgM binding, so the suppression is not a reflection of decreased IgM binding. Importantly, these data show translation of the suppressive effect of Siglec-2 Ligand conjugates between a primary human B cell system and those shown in a primary mouse B cell system.

### Example 7: In Vivo Suppression of Anti-Drug Antibody in Mice Treated with Siglec Ligand-Adalimumab Conjugates at LDR 10

### Purpose

The purpose of this experiment was to test for suppression of immunogenicity in mice dosed with adalimumab-Siglec-2 ligand conjugates. Parental adalimumab hIgG1 is highly immunogenic in mice, with a strong immunoglobulin response after a single dose. This and subsequent examples set out to corroborate the in vitro B cell suppressive effects shown in Examples 5 and 6 with in vivo assessment of effects on immunogenicity for Siglec Ligand conjugates with different proteins. This initial experiment served as a dose-range finding study for a few select ligands with a range of affinities.

### Materials and Methods

Adalimumab and Adalimumab-Siglec Ligand conjugates were prepared as described in Example 4 with pentafluorophenyl (PFP) conjugation to native lysine residues to an LDR of 10.

To evaluate the production of antibodies specific to adalimumab and/or adalimumab-Siglec Ligand conjugates, C57BL/6 mice were immunized through subcutaneous injections with adalimumab or CD22 ligand-conjugated adalimumab. On study day −1, animals were randomized into treatment groups based on body weight. On study day 0, animals were bled for baseline serum and then injected IV with adalimumab or the adalimumab-Siglec Ligand conjugates. The individual antigens were prepared by making antigen solution in sterile PBS pH 7.4. Animals were then injected with 0.2 mL of the 1.25 mg/mL, 0.375 mg/mL, or 0.0375 mg/mL antigen subcutaneously weekly (days 0, 7, 14, 21) with 0.1 mL on each side of the rear flank near base of tail. The total dose based on a 25 g mouse would be 10, 3, or 0.3 mg/kg. Animals were bled via the retro-orbital sinus weekly (days 6, 13, 20) throughout the study under inhaled isoflurane anesthesia. On study day 28, animals were anesthetized with inhaled isoflurane anesthesia and then bled via cardiac puncture and then sacrificed by cervical dislocation. Whole blood was collected into Microvette EDTA capillary collection tubes (Sarstedt Inc) and then further processed following the manufacturer's instructions for serum collection. Samples were stored at −80C until analysis was performed.

ADA assays were performed on 96-well assay plates (Nunc Plates, Black 96-Well Immuno Plates, Thermo Scientific, Cat. No. 437111) coated with antigen, as follows. A mixture of adalimumab and adalimumab conjugates was coated at 5 μg/ml of each, with 100 μL/well. All coated antigens were diluted in PBS pH 7.2 and incubated overnight at 4° C. The following day, plate coating solution was removed, and plates were blocked with 200 μL/well of 3% BSA, 20 μM EDTA, 0.1% Tween-20 in PBS for 1 hour at room temperature. Serum samples were diluted 1:185 in 3% BSA, 20 μM EDTA, 0.1% Tween-20 in PBS and added in three-fold serial dilutions. Plates were incubated 1 hour at room temperature, then washed with PBS buffer with 0.05% Tween-20. After washing, 100 μL of 1:2500 diluted Donkey Anti-Mouse IgG(H+L)-HRP (SouthernBiotech, Cat. No. 6411-05) was added and incubated for 1 hour at room temperature. After washing the assay plates, 100 μL of QuantaBlu Substrate Solution (Thermo Scientific, Cat. No. 15169) was added to each well and incubated for 15 minutes. The excitation and emission settings for the QuantaBlu Fluorogenic Peroxide Substrates are 325 nm and 420 nm and the relative florescence units were measured using a SpectraMax plate reader. Serum dilution curves were generated for days 7, 14, 21, and 28. The titer cut point was determined by calculating the mean of the naïve, untreated mice and adding twice the standard deviation of the untreated samples.

### Results and Conclusions

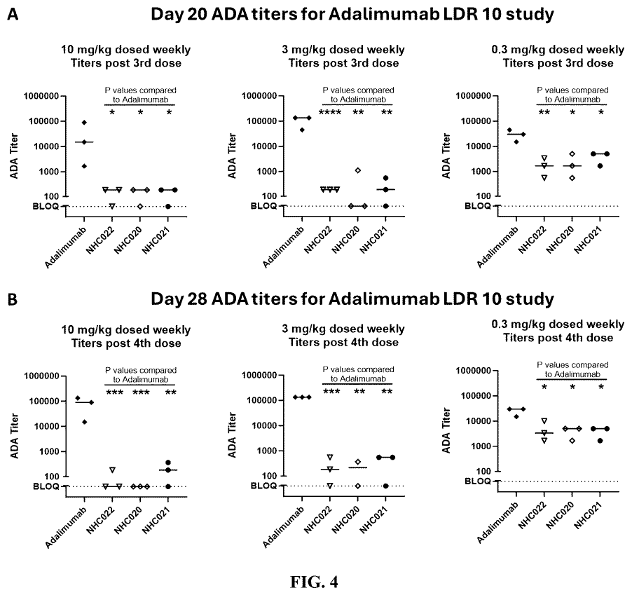

FIG. 4 shows the ADA titers of mice dosed at 10, 3, and 0.3 mg/kg at day 20, post 3^{rd} dose (FIG. 4A) and day 27, post 4^{th} dose (FIG. 4B). Adalimumab clearly shows high ADA titers at all concentrations and timepoints. In contrast, all of the CD22-ligand Adalimumab conjugates show significantly lower ADA titers at all concentrations and timepoints. The 10 and 3 mg/kg doses demonstrated the lowest ADA titers, with many of the mice measuring below the limit of quantification for the ADA assay. These results for suppression of immunogenicity in mice correlate with the in vitro B cell activation results in Examples 5 and 6; decoration of an immunogenic antibody with potentiated CD22 ligand is sufficient to strongly suppress immunogenicity.

### Example 8: Pharmacokinetic Analysis of Adalimumab and Siglec Ligand-Conjugated Adalimumab in C57BL/6 Mice

### Purpose

The purpose of this experiment was to determine if there are improvements in exposure and pharmacokinetics of Siglec Ligand-conjugate adalimumab preparations relative to parental adalimumab IgG.

### Materials and Methods

The Adalimumab and Adalimumab CD22 ligand conjugates were prepared as described in Example 4. The conjugates in this study were conjugated via maleimide coupling to engineered cysteine site L443C on the heavy chain for a final LDR of 2. Control adalimumab was cysteine capped at L443C with N-ethylmaleimide (NEM)

Pharmacokinetic monitoring was carried out in C57BL/6 mice following a single intravenous administration of adalimumab or the individual SigL-conjugated adalimumab antibodies. On study day−1, the mice were randomized by body weight into treatment groups. The individual antigens were prepared by making antigen solution in sterile PBS pH 7.4. On study day 0, the animals were administered the test articles subcutaneously twice with 0.1 mL each of a 0.5 mg/mL antigen solution for a total dose of approximately 4 mg/kg. Animals were bled via the retro-orbital sinus throughout the study under inhaled isoflurane anesthesia at 1 h, 6 h, 1-day, 2-days, 6-days, 13-days, 20 days and 27-days post test-article administration. Whole blood was collected into Microvette K2EDTA capillary collection tube (Sarstedt Inc) and then further processed following manufactures instructions to collect plasma. Levels of adalimumab and SigL-conjugated adalimumab in plasma samples were measured using an AlphaLISA human IgG assay (Perkin Elmer) following manufacturer's protocols.

### Results and Conclusions

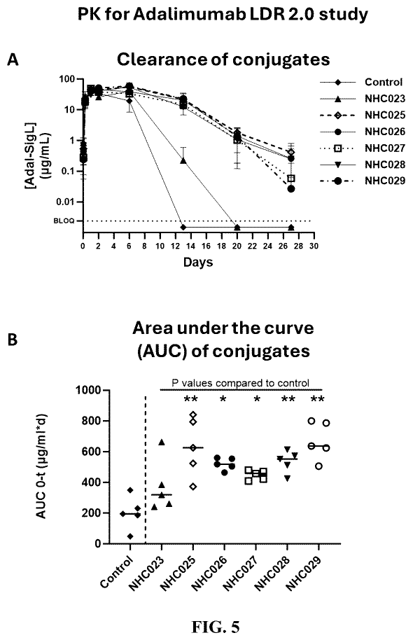

FIG. 5 depicts analysis of serum pharmacokinetics for adalimumab and CD22-ligand-adalimumab conjugates in mice. Clearly, the control adalimumab displays significantly increased clearance, falling below the limit of quantitation (BLOQ) at day 14 in FIG. 5A. This is likely due to the high ADA titers observed with adalimumab, as shown in Example 7. Interestingly, adalimumab CD22 ligand conjugate NHC023 also shows significant accelerated clearance. NHC023 is conjugated to a CD22 ligand that shows the lowest affinity to Siglec-2 of the group of CD22 ligands that were assessed in this study. This is likely a measure of ADA suppression as well, since in vitro studies from Examples 5 and 6 demonstrated higher affinity CD22 ligands are better suppressors of B-cell activation than lower affinity ligands.

FIG. 5B depicts the area under the curve (AUC) for each mouse examined in the PK assay. The higher affinity ligands that are conjugated to adalimumab, NHC025 to NHC029, demonstrate significantly better exposure over time. Overall, this study establishes that CD22 ligand conjugates to adalimumab can rescue accelerated drug clearance associated with ADA.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) is not invoked.

1. A Siglec ligand of formula (I): 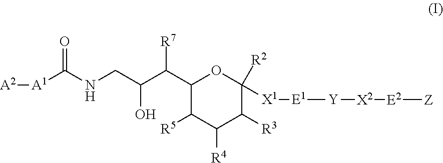
wherein:
A¹ is absent or alkylene;
A² is absent, alkyl, aryl, heteroaryl, cycloalkyl, or a substituted version thereof;
R² is —COOH, —P(O)(OH)₂, —OSO₂OH, —C(O)NHSO₂H, tetrazole, an ester, or a substituted version thereof;
R³ is H, alkyl, alkoxy, —S(alkyl), amino, halo, cyano, ester, amide, or a substituted version thereof;
R⁴ is hydroxy, alkoxy, amino, amide or a substituted version thereof;
R⁵ is H, amino, ureido, amide, thioamide, oxamide, —NH(cyclobut-3-ene-1,2-dione), sulfonaamide, phosphoramide, alkylamine, triazole, tetrazole, or substituted version thereof;
R⁷ is hydroxy or halo;
X¹ is O, S, triazole, —CH(OH)—, —CH₂—, —CHF—, —NH—, or —CF₂—;
E¹ is absent, —(CH₂CH₂O)_{g}—, —(CH₂)_{g}—, benzyl, cycloalkyl, or a substituted version thereof, wherein g is an integer ranging from 1 to 10;
Y is absent, alkylene, phenylene, substituted phenylene, —O—, —NH—, —S—, —C≡C—, —CH₂C≡C—, —C(O)—, —OP(O)O—, or —NH-(cyclobut-3-ene-1,2-dione)-NH—, —NHC(O)—, triazole, —(CH₂CH₂O)ₘ—, —(CH₂)ₘ—, wherein m is an integer ranging from 1 to 10;
X² is absent, O, —NH—, —C(O)NH—, —C(O)—, —C(O)O—, —NHC(O)—, —NHC(O)O—, —OP(O)(OH)O—, —NHC(O)NH—, —(CH₂)ₙ—, —(CH₂CH₂O)ₙ—, —(CH₂CH₂O)ₙNHC(O)—, triazole, or a substituted version thereof, wherein n is an integer ranging from 1 to 10;
E² is absent, —(CH₂)ₕ—, —(CH₂CH₂O)ₕ—, —(CH₂CH₂O)ₕC(O)NH—, -(alkylene)C(O)NH(alkylene)-, (alkylene)C(O)NH(substituted alkylene)-, -(alkylene)C(O)N(substituted alkylene)₂-, —C≡C—, or —CH₂C≡C—, or a substituted version thereof, wherein h is an integer ranging from 1 to 10; and
Z is Z1 or Z1′-T-Z2, wherein Z1 is a chemoselective functional group, wherein Z1′ is a diradical group, T is absent or a diradical group, and Z2 is a chemoselective functional group,
a salt thereof, or a stereoisomer thereof.

2. The Siglec ligand of claim 1, wherein:
A¹ is absent or alkylene;
A² is absent, aryl, heteroaryl, cycloalkyl, or a substituted version thereof;
R² is —COOH, —P(O)(OH)₂, —OSO₂OH, —C(O)NHSO₂H, tetrazole, an ester, or a substituted version thereof;
R³ is H, alkoxy, amino, or a substituted version thereof;
R⁴ is hydroxy, alkoxy, amino, amide, or a substituted version thereof;
R⁵ is amino, ureido, amide, thioamide, oxamide, —NH(cyclobut-3-ene-1,2-dione), sulfonamide or substituted version thereof;
R⁷ is hydroxy or halo;
X¹ is O, S, or triazole;
E¹ is absent, —(CH₂CH₂O)_{g} or —(CH₂)_{g}—, or a substituted version thereof, wherein g is an integer ranging from 1 to 10;
Y is alkylene, phenylene, or substituted phenylene;
X² is absent or O; and
E² is absent, —(CH₂)ₕ—, -(alkylene)C(O)NH(alkylene)-, (alkylene)C(O)NH(substituted alkylene)-, or -(alkylene)C(O)N(substituted alkylene)₂-, wherein h is an integer ranging from 1 to 10.

3. A Siglec conjugate of formula (X): 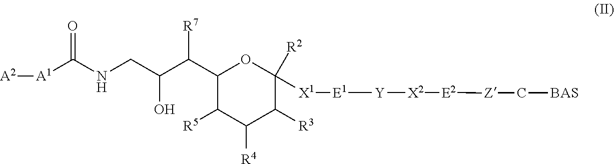
wherein:
A¹ is absent or alkylene;
A² is absent, aryl, heteroaryl, cycloalkyl, or a substituted version thereof;
R² is —COOH, —P(O)(OH)₂, —OSO₂OH, —C(O)NHSO₂H, tetrazole, an ester, or a substituted version thereof;
R³ is H, alkoxy, —S(alkyl), amino, halo, cyano, ester, amide, or a substituted version thereof;
R⁴ is hydroxy, alkoxy, amino, amide, or a substituted version thereof;
R⁵ is H, amino, ureido, amide, thioamide, oxamide, —NH(cyclobut-3-ene-1,2-dione), sulfonaamide, phosphoramide, alkylamine, triazole, tetrazole, or substituted version thereof;
R⁷ is hydroxy or halo;
X¹ is O, S, triazole, —CH(OH)—, —CH₂—; —CHF—, —NH—, or —CF₂—;
E¹ is absent, —(CH₂CH₂O)_{g}, —(CH₂)_{g}—, benzyl, cycloalkyl, or a substituted version thereof, wherein g is an integer ranging from 1 to 10;
Y is absent, alkylene, phenylene, substituted phenylene, —O—, —NH—, —S—, —C≡C—, —CH₂C≡C—, —C(O)—, —OP(O)O—, or —NH-(cyclobut-3-ene-1,2-dione)-NH—, —NHC(O)—, triazole, —(CH₂CH₂O)_{g}—, —(CH₂)_{g}—, wherein g is an integer ranging from 1 to 10;
X² is absent, O, —NH—, —C(O)NH—, —C(O)—, —C(O)O—, —NHC(O)—, —NHC(O)O—, —OP(O)(OH)O—, —NHC(O)NH—, —(CH₂)_{g}—, —(CH₂CH₂O)_{g}—, —(CH₂CH₂O)_{g}NHC(O)—, triazole, or a substituted version thereof, wherein g is an integer ranging from 1 to 10;
E² is absent, —(CH₂)ₕ—, —(CH₂CH₂O)ₕ, —(CH₂CH₂O)ₕC(O)NH—, -(alkylene)C(O)NH(alkylene)-, (alkylene)C(O)NH(substituted alkylene)-, -(alkylene)C(O)N(substituted alkylene)₂-, —C≡C—, or —CH₂C≡C—, or a substituted version thereof, wherein h is an integer ranging from 1 to 10; and
Z′ is Z1′ or Z1′-T-Z2′, wherein Z1′ is a diradical group, T is absent or a diradical group, and Z2′ is a diradical group;
C is absent or a diradical group; and
BAS is a biologically active substance,
a salt thereof, or a stereoisomer thereof.

4. The Siglec conjugate of claim 3, wherein:
A¹ is absent or alkylene;
A² is absent, aryl, heteroaryl, cycloalkyl, or a substituted version thereof;
R² is —COOH, —P(O)(OH)₂, —OSO₂OH, —C(O)NHSO₂H, tetrazole, an ester, or a substituted version thereof;
R³ is H, alkoxy, amino, or a substituted version thereof;
R⁴ is hydroxy, alkoxy, amino, amide, or a substituted version thereof;
R⁵ is amino, ureido, amide, thioamide, oxamide, —NH(cyclobut-3-ene-1,2-dione), sulfonamide or substituted version thereof;
R⁷ is hydroxy or halo;
X¹ is O, S, or triazole;
E¹ is absent, —(CH₂CH₂O)_{g}— or —(CH₂)_{g}—, or a substituted version thereof, wherein g is an integer ranging from 1 to 10;
Y is alkylene, phenylene, or substituted phenylene;
X² is absent, or O; and
E² is absent, —(CH₂)ₕ—, -(alkylene)C(O)NH(alkylene)-, -(alkylene)C(O)N(substituted alkylene)₂-, or (alkylene)C(O)NH(substituted alkylene)-, wherein h is an integer ranging from 1 to 10.

5. A Siglec ligand of formula (XI): 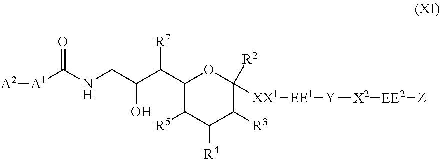
wherein:
A¹ is absent or alkylene;
A² is absent, alkyl, aryl, heteroaryl, cycloalkyl, or a substituted version thereof;
R² is —COOH, —P(O)(OH)₂, —OSO₂OH, —C(O)NHSO₂H, tetrazole, an ester, or a substituted version thereof;
R³ is H, alkyl, alkoxy, —S(alkyl), amino, halo, cyano, ester, amide, or a substituted version thereof;
R⁴ is hydroxy, alkoxy, amino, amide, or a substituted version thereof;
R⁵ is H, amino, ureido, amide, thioamide, oxamide, —NH(cyclobut-3-ene-1,2-dione), sulfonaamide, phosphoramide, alkylamine, triazole, tetrazole, or substituted version thereof;
R⁷ is hydroxy or halo;
XX¹ is S, triazole, —CH(OH)—, —CH₂—, —CHF—, —NH—, or —CF₂—;
EE¹ is absent, —(CH₂CH₂O)_{f}—, —(CH₂)_{g}—, benzyl, cycloalkyl, or a substituted version thereof, wherein f and g are each independently an integer ranging from 1 to 10;
Y is absent, alkylene, phenylene, substituted phenylene, —O—, —NH—, —S—, —C≡C—, —CH₂C≡C—, —C(O)—, —OP(O)O—, —NH-(cyclobut-3-ene-1,2-dione)-NH—, —NHC(O)—, triazole, —(CH₂CH₂O)_{g}—, —(CH₂)_{g}—, wherein g is an integer ranging from 1 to 10;
X² is absent, O, —NH—, —C(O)NH—, —C(O)—, —C(O)O—, —NHC(O)—, —NHC(O)O—, —OP(O)(OH)O—, —NHC(O)NH—, —(CH₂)_{g}—, —(CH₂CH₂O)_{g}—, —(CH₂CH₂O)_{g}NHC(O)—, triazole, or a substituted version thereof, wherein g is an integer ranging from 1 to 10;
EE² is absent, —(CH₂)ₕ—, -(alkylene)C(O)NH(alkylene)-, (alkylene)C(O)NH(substituted alkylene)-, -(alkylene)C(O)N(substituted alkylene)₂-, —C≡C—, —CH₂C≡C—, —(CH₂CH₂O)ᵢ— or —(CH₂CH₂O)ₕC(O)NH—, wherein h and i are each independently selected from an integer ranging from 1 to 10; and
Z is Z1 or Z1′-T-Z2, wherein Z1 is a chemoselective functional group, wherein Z1′ is a diradical group, T is absent or a diradical group, and Z2 is a chemoselective functional group,
a salt thereof, or a stereoisomer thereof.

6. The Siglec ligand of claim 5, wherein:
A¹ is absent or alkylene;
A² is absent, aryl, heteroaryl, cycloalkyl, or a substituted version thereof;
R² is —COOH, —P(O)(OH)₂, —OSO₂OH, —C(O)NHSO₂H, tetrazole, an ester, or a substituted version thereof;
R³ is H, alkoxy, amino, or a substituted version thereof;
R⁴ is hydroxy, alkoxy, amino, amide, or a substituted version thereof;
R⁵ is amino, ureido, amide, thioamide, oxamide, —NH(cyclobut-3-ene-1,2-dione), sulfonamide or substituted version thereof;
R⁷ is hydroxy or halo;
XX¹ is S, triazole;
EE¹ is absent, —(CH₂CH₂O)_{f}—, or —(CH₂)_{g}—, or a substituted version thereof, wherein f and g are each independently an integer ranging from 1 to 10;
Y is alkylene, phenylene, or substituted phenylene;
X² is absent or O; and
EE² is absent, —(CH₂)ₕ—, -(alkylene)C(O)NH(alkylene)-, (alkylene)C(O)NH(substituted alkylene)-, -(alkylene)C(O)N(substituted alkylene)₂-, or —(CH₂CH₂O)ᵢ—, wherein h and i are each independently selected from an integer ranging from 1 to 10.

7. A Siglec conjugate of formula (XII): 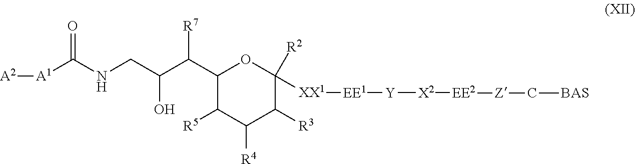
wherein:
A¹ is absent or alkylene;
A² is absent, alkyl, aryl, heteroaryl, cycloalkyl, or a substituted version thereof;
R² is —COOH, —P(O)(OH)₂, —OSO₂OH, —C(O)NHSO₂H, tetrazole, an ester, or a substituted version thereof;
R³ is H, alkyl, alkoxy, —S(alkyl), amino, halo, cyano, ester, amide, or a substituted version thereof;
R⁴ is hydroxy, alkoxy, amino, amide, or a substituted version thereof;
R⁵ is H, amino, ureido, amide, thioamide, oxamide, —NH(cyclobut-3-ene-1,2-dione), sulfonaamide, phosphoramide, alkylamine, triazole, tetrazole, or substituted version thereof;
R⁷ is hydroxy or halo;
XX¹ is S, triazole, —CH(OH)—, —CH₂—, —CHF—, —NH—, or —CF₂—;
EE¹ is absent, —(CH₂CH₂O)_{f}—, —(CH₂)_{g}—, benzyl, cycloalkyl, or a substituted version thereof, wherein f and g are each independently an integer ranging from 1 to 10;
Y is absent, alkylene, phenylene, substituted phenylene, —O—, —NH—, —S—, —C≡C—, —CH₂C≡C—, —C(O)—, —OP(O)O—, —NH-(cyclobut-3-ene-1,2-dione)-NH—, —NHC(O)—, triazole, —(CH₂CH₂O)_{g}—, —(CH₂)_{g}—, wherein g is an integer ranging from 1 to 10;
X² is absent, O, —NH—, —C(O)NH—, —C(O)—, —C(O)O—, —NHC(O)—, —NHC(O)O—, —OP(O)(OH)O—, —NHC(O)NH—, —(CH₂)_{g}—, —(CH₂CH₂O)_{g}—, —(CH₂CH₂O)_{g}NHC(O)—, triazole, or a substituted version thereof, wherein g is an integer ranging from 1 to 10;
EE² is absent, —(CH₂)ₕ—, -(alkylene)C(O)NH(alkylene)-, (alkylene)C(O)NH(substituted alkylene)-, -(alkylene)C(O)N(substituted alkylene)₂-, —C≡C—, —CH₂C≡C—, —(CH₂CH₂O)ᵢ—, —(CH₂CH₂O)ₕC(O)NH—, or a substituted version thereof, wherein h and i are each independently selected from an integer ranging from 1 to 10; and
Z′ is Z1′ or Z1′-T-Z2′, wherein Z1′ is a diradical group, T is absent or a diradical group, and Z2′ is a diradical group;
C is absent or a diradical group; and
BAS is a biologically active substance,
a salt thereof, or a stereoisomer thereof.

8. The Siglec conjugate of claim 7, wherein:
A¹ is absent or alkylene;
A² is absent, aryl, heteroaryl, cycloalkyl, or a substituted version thereof;
R² is —COOH, —P(O)(OH)₂, —OSO₂OH, —C(O)NHSO₂H, tetrazole, an ester, or a substituted version thereof;
R³ is H, alkoxy, amino, or a substituted version thereof;
R⁴ is hydroxy, alkoxy, amino, amide, or a substituted version thereof;
R⁵ is amino, ureido, amide, thioamide, oxamide, —NH(cyclobut-3-ene-1,2-dione), sulfonamide or substituted version thereof;
R⁷ is hydroxy or halo;
X¹ is S or triazole;
E¹ is absent, —(CH₂CH₂O)_{f}—, or —(CH₂)_{g}—, or a substituted version thereof, wherein f and g are each independently an integer ranging from 1 to 10;
Y is alkylene, phenylene, or substituted phenylene;
X² is absent or O; and
EE² is absent, —(CH₂)ₕ—, -(alkylene)C(O)NH(alkylene)-, (alkylene)C(O)NH(substituted alkylene)-, -(alkylene)C(O)N(substituted alkylene)₂-, or —(CH₂CH₂O)ᵢ—, wherein h and i are each independently selected from an integer ranging from 1 to 10.

9. The Siglec conjugate of claim 3, wherein:
X¹ is O or S;
E¹ is —(CH₂)_{g}—; and
Y is phenylene or substituted phenylene.

10. The Siglec conjugate of claim 3, wherein:
X¹ is O or S;
E¹ is —(CH₂)_{g}—;
Y is phenylene or substituted phenylene; and
X² is O.

11. The Siglec conjugate of claim 3, wherein:
X¹ is O or S;
E¹ is —(CH₂)_{g}—;
Y is phenylene or substituted phenylene;
X² is O; and
E² is —(CH₂)ₕ—.

12. The Siglec conjugate of claim 3, wherein g is 1, 2, or 3.

13. The Siglec conjugate of claim 3, wherein h is 1, 2, or 3.

14. The Siglec conjugate of claim 3, wherein X¹ is O or S.

15. The Siglec conjugate of claim 3, wherein X¹ is O.

16. The Siglec conjugate of claim 3, wherein X¹ is triazole.

17. The Siglec conjugate of claim 3, wherein E² is —(CH₂)ₕ—.

18. The Siglec conjugate of claim 3, wherein E² is -(alkylene)C(O)NH(alkylene)- or (alkylene)C(O)NH(substituted alkylene)-.

19. The Siglec conjugate of claim 3, wherein Y is phenylene or substituted phenylene.

20. The Siglec conjugate of claim 3, wherein Z is selected from the group consisting of alkyne, azide, thiol, maleimide, N-substituted maleimide, iodoacetamide, amine, carboxylic acid or active ester thereof, alkyne, tetrazine, trans-cyclooctene, diene, dienophile, hydroxyl, hydrazido, hydrazino, aldehyde, ketone, azido, phosphine, epoxide, succinimide, aryl, substituted aryl, tetrahydropyran, 5-[(3AS,4R,6AR)-2-Oxohexahydro-1H-thieno[3,4-D]imidazol-4-YL]pentanoic acid, pentafluorophenyl ester, amide, alkyl, and phosphate.

21. The Siglec conjugate of claim 3, wherein Z is selected from the group consisting of alkyne, azide, thiol, maleimide, N-substituted maleimide, carboxylic acid or active ester thereof, tetrazine, trans-cyclooctene, diene, dienophile, aryl, substituted aryl, tetrahydropyran, 5-[(3AS,4R,6AR)-2-Oxohexahydro-1H-thieno[3,4-D]imidazol-4-YL]pentanoic acid, pentafluorophenyl ester, amide, alkyl, or phosphate.

22. The Siglec conjugate of claim 3, wherein Z′ is triazole.

23. The Siglec conjugate of claim 3, wherein one or both of A¹ and A² are present.

24. The Siglec conjugate of claim 3, wherein A¹ is —CH₂—.

25. The Siglec conjugate of claim 3, wherein A² is aryl, heteroaryl, or a substituted version thereof.

26. The Siglec conjugate of claim 3, wherein A² is biphenyl, phenylpyridine, phenyl, or a substituted version thereof.

27. The Siglec conjugate of claim 3, wherein R² is —COOH or an ester thereof.

28. The Siglec conjugate of claim 3, wherein R³ is H.

29. The Siglec conjugate of claim 3, wherein R⁴ is hydroxy.

30. The Siglec conjugate of claim 3, wherein R⁵ is —NHC(O)H, —NHC(O)NH₂, or —NHC(O)CH₂OH.

31. The Siglec conjugate of claim 3, wherein R⁵ is —NHC(O)CH₂OH.

32. The Siglec conjugate of claim 3, wherein R⁷ is hydroxy.

33. The Siglec conjugate of claim 3, wherein:
A¹ is absent;
A² is alkyl;
R² is —COOH;
R³ is H;
R⁴ is hydroxy;
R⁵ is ureido;
R⁷ is hydroxy.

34. The Siglec conjugate of claim 3, wherein:
A¹ is absent;
A² is substituted alkyl;
R² is tetrazole;
R³ is H;
R⁴ is hydroxy;
R⁵ is amide;
R⁷ is hydroxy.

35. The Siglec conjugate of claim 3, wherein:
A¹ is absent;
A² is substituted alkyl;
R² is —C(O)NHSO₂H;
R³ is H;
R⁴ is hydroxy;
R⁵ is oxamide;
R⁷ is hydroxy.

36. A pharmaceutical composition comprising:
a Siglec conjugate according to claim 3; and
a pharmaceutical excipient.

37. A method of making a Siglec conjugate, the method comprising:
covalently attaching a Siglec ligand of claim 1 to a biologically active substance (BAS),
thereby making the conjugate.

38. (canceled)

39. The method of claim 37, wherein the covalently attaching comprises sialylation by chemical conjugation.

40.-42. (canceled)

43. The method of claim 39, wherein the chemical conjugation of the sialic acid is to an amino acid of the biologically active substance.

44. The method of claim 39, wherein the chemical conjugation of the sialic acid to the amino acid of the biologically active substance results in a covalent bond between the sialic acid and the amino acid.

45. The method of claim 39, wherein the chemical conjugation of the sialic acid to the amino acid of the biologically active substance incorporates a connector between the sialic acid and the amino acid.

46. (canceled)

47. The method of claim 39, wherein the sialic acid is a non-naturally occurring sialic acid.

48.-49. (canceled)

50. The method of claim 37, wherein the covalently attaching results in the generation of a Siglec ligand.

51. The method of claim 37, wherein the Siglec ligand is a ligand for a B cell-associated Siglec.

52. The method of claim 51, wherein the B-cell associated Siglec is selected from the group consisting of Siglec-2 (CD22), Siglec-5 (CD170), Siglec-6, Siglec-9 (CD329) and Siglec-10 (Siglec G).

53. The method of claim 37, wherein the amount of sialic acid associated with the biologically active substance is increased 2-fold or more following the covalent attaching.

54-56. (canceled)

57. The method of claim 37, wherein the BAS is a protein.

58. The method of claim 57, wherein the protein is selected from the group consisting of an antibody, an enzyme, a chimeric protein, and a viral particle.

59. The method of claim 58, wherein the antibody is selected from the group consisting of a monoclonal antibody, a bispecific antibody, an scFv, a Fab, a camelid, or a nanobody.

60. The method of claim 58, wherein the antibody is selected from the group consisting of adalimumab, infliximab, cetuximab, natalizumab, moxetumomab pasudotox, atezolizumab, nivolumab, abciximab, Brentuximab, Certolizumab pegol, elotuzumab, benralizumab, vedolizumab, galcanezumab, rituximab, alemtuzumab, dupilumab, golimumab, obinutuzumab, tildrakizumab, erenumab, mepolizumab, tamucirumab, ranibizumab, ustekinumab, reslizumab, ipilimumab, alirocumab, belimumab, panitumumab, avelumab, necitumumab, mogamulizumab, olaratumab, brodalumab, eculizumab, pertuzumab, pembrolizumab, and tocilizumab.

61. The method of claim 57, wherein the protein is selected from the group consisting of erythropoietin, thrombopoietin, human growth hormone, tissue factor, IFNβ-1b, IFNβ-1a, IL-2 or the IL-2 mimetic aldesleukin, exenatide, albiglutide, alefacept, palifermin, and belatacept.

62. The method of claim 58, wherein the enzyme is selected from the group consisting of asparaginase Erwinia chrysanthemi, phenylalanine ammonia-lyase, alpha-galactosidase A, acid α-glucosidase (GAA), glucocerebrosidase (GCase), aspartylglucosaminidase (AGA), alpha-L-iduronidase, iduronate sulfatase, sulfaminase, α-N-acetylglucosaminidase (NAGLU), heparin acetyle CoA: α-glucosaminide N-acetyltransferase (HGSNAT), N-acetylglucosamine 6-sulfatase (GNS), N-glucosamine 3-O-sulfatase (arylsulfatase G or ARSG), N-acetylgalactosamine 6-sulfatase, beta-galactosidase, N-acetylgalactosamine 4-sulfatase, beta-glucuronidase, Factor VIII, Factor IX, palmitoyl protein thioesterase (PPT1), and Tripeptidyl peptidase (TPP1).

63. The method of claim 58, wherein the viral particle is selected from a recombinant adeno-associated virus (rAAV) particle, a recombinant human adenovirus (rHAdV) particle, a recombinant Herpes Simplex Virus (rHSV) particle, a recombinant papillomavirus (PV) particle, a recombinant polyomavirus particle, a recombinant vaccinia virus particle, a recombinant cytomegalovirus (CMV) particle, a recombinant baculovirus particle, a recombinant human papillomavirus (HPV) particle, and a recombinant retrovirus particle.

64. A method of treating a patient for a condition, the method comprising:
administering to the patient a conjugate according to claim 3.

65. The method of claim 64, wherein the condition is a chronic immune disease selected from the group consisting of rheumatoid arthritis, psoriatic arthritis, ankylosing spondylitis, Crohn's disease, ulcerative colitis, psoriasis, hidradenitis suppurativa, uveitis, and juvenile idiopathic arthritis, wherein the administering comprises administering to the individual an engineered hypoimmunogenic TNFα-specific antibody selected from adalimumab and infliximab in an amount effective to treat the chronic immune disease.

66. The method of claim 64, wherein the condition is a leukemia, wherein the BAS is asparaginase from Erwinia chrysanthemi.

67. (canceled)

68. The method of claim 64, wherein the condition is an antibody response to transplanted tissue, wherein the BAS is IdeS.

69. The method of claim 68, wherein the transplanted tissue is an allogeneic graft.

70. The method of claim 68, wherein the transplanted tissue is a xenograft.

71. The method of claim 68, wherein the transplanted tissue is selected from kidney, heart, lung, liver, pancreas, trachea, vascular tissue, skin, bone, cartilage, adrenal tissue, fetal thymus, and cornea.

72. (canceled)

73. The method of claim 64, wherein the condition is enzyme deficiency and the BAS is the deficient enzyme.

74. The method of claim 73, wherein the enzyme deficiency is a deficiency for an enzyme selected from the group consisting of phenylalanine ammonia-lyase (PKU), alpha-galactosidase A (for Fabry), acid α-glucosidase (GAA, for Pompe), glucocerebrosidase (GCase, for Gaucher), aspartylglucosaminidase (AGA, for Aspartylglucosaminuria), alpha-L-iduronidase (for MPS 1), iduronate sulfatase (for MPS II), sulfaminase (MPS IIIa), α-N-acetylglucosaminidase (NAGLU, for MPS IIIB), heparin acetyle CoA: α-glucosaminide N-acetyltransferase (HGSNAT, for MPS IIIC), N-acetylglucosamine 6-sulfatase (GNS, for MPS IIID), N-glucosamine 3-O-sulfatase (arylsulfatase G or ARSG, MPS IIIE), N-acetylgalactosamine 6-sulfatase (for MPS IVA), beta-galactosidase (for MPS IVB), N-acetylgalactosamine 4-sulfatase (for MPS VI), beta-glucuronidase (for MPS VI), Factor VIII (for hemophilia A), Factor IX (for hemophilia B), palmitoyl protein thioesterase (PPT1, for CLN1), Tripeptidyl peptidase (TPP1, for CLN2), and cystathionine beta synthase (CBS) deficiency.

75. The method of claim 64, wherein the condition is a monogenic disease, wherein the BAS is a viral particle comprising a transgene encoding a therapeutic product.

76. The method of claim 64, wherein the BAS is a biotherapeutic, wherein the method further comprises:
drawing serum from the individual 8 weeks after administering the biotherapeutic conjugate and assessing the serum for biotherapeutic-specific antibodies, wherein the titer of biotherapeutic-specific antibodies is 50% of the titer that would be elicited by a corresponding unengineered biotherapeutic.

77. The method of claim 76, wherein the titer of biotherapeutic-specific antibodies is 20% of the titer that would be elicited by a corresponding unengineered biotherapeutic.

78. The method of claim 76, wherein the titer of biotherapeutic-specific antibodies is 5% of the titer that would be elicited by a corresponding unengineered biotherapeutic.

79. The method of claim 76, wherein biotherapeutic-specific antibodies cannot be detected.
